(19) United States
(12) Patent Application Publication
(10) Pub. No.: US 2024/0368120 A1
(43) Pub. Date: Nov. 7, 2024

(54) COMPOUNDS AS GLP-1R AGONISTS

(71) Applicant: Terns Pharmaceuticals, Inc., Foster City, CA (US)

(72) Inventors: Gary W. LUEHR, Foster City, CA (US); Christopher T. JONES, Foster City, CA (US); Kevin P. QUINN, Foster City, CA (US)

(21) Appl. No.: 18/622,057

(22) Filed: Mar. 29, 2024

Related U.S. Application Data
(60) Provisional application No. 63/610,244, filed on Dec. 14, 2023.
(60) Provisional application No. 63/587,861, filed on Oct. 4, 2023.
(60) Provisional application No. 63/579,257, filed on Aug. 28, 2023.
(60) Provisional application No. 63/492,895, filed on Mar. 29, 2023.

(51) Int. Cl. C07D 401/14; A61K 31/4439; A61K 31/454; A61K 31/4545; A61K 31/4725; A61K 31/496; A61K 31/497; A61K 31/501; A61K 31/506; C07D 401/06; C07D 401/10; C07D 401/12; C07D 405/14; C07D 409/14; C07D 413/14; C07D 417/14; C07D 471/04

(52) U.S. Cl. CPC C07D 401/14; A61K 31/4439; A61K 31/454; A61K 31/4545; A61K 31/4725; A61K 31/496; A61K 31/497; A61K 31/501; A61K 31/506; C07D 401/06; C07D 401/10; C07D 401/12; C07D 405/14; C07D 409/14; C07D 413/14; C07D 417/14; C07D 471/04

(57) ABSTRACT

The present application provides compounds that may be used as a glucagon-like peptide-1 receptors (GLP-1R) agonist, or pharmaceutically acceptable salts thereof. Also provided are pharmaceutical compositions containing such compounds, or pharmaceutically acceptable salts thereof. Methods of preparing these compounds and compositions, and methods of using these compounds and compositions to treat or prevent a disease or a condition mediated by GLP-1R, are also provided.

### CROSS-REFERENCE TO RELATED APPLICATIONS

This application claims priority to, and the benefit of, U.S. Provisional Application No. 63/610,244, filed on Dec. 14, 2023, U.S. Provisional Application No. 63/587,861, filed on Oct. 4, 2023, U.S. Provisional Application No. 63/579,257, filed on Aug. 28, 2023, and U.S. Provisional Application No. 63/492,895, filed on Mar. 29, 2023, the entire contents of each of which are incorporated herein by reference.

### BACKGROUND

GLP-1 is a 30 amino acid long incretin hormone secreted by the L-cells in the intestine in response to ingestion of food. GLP-1 has been shown to stimulate insulin secretion in a physiological and glucose-dependent manner, decrease glucagon secretion, inhibit gastric emptying, decrease appetite, and stimulate proliferation of beta-cells. Marketed GLP-1R agonists are peptides that are typically administered by subcutaneous injection. Liraglutide and semaglutide were the first GLP-1 peptides to be approved for both the treatment of type II diabetes mellitus (T2DM) and obesity. Semaglutide has also been approved for the treatment of T2DM as a bioavailable oral formulation.

In a healthy individual, GLP-1 plays an important role regulating post-prandial blood glucose levels by stimulating glucose-dependent insulin secretion by the pancreas resulting in increased glucose absorption in the periphery. GLP-1 also suppresses glucagon secretion, leading to reduced hepatic glucose output. In addition, GLP-1 delays gastric emptying and slows small bowel motility delaying food absorption.

Development of an oral GLP-1R agonist with favorable PK properties is an outstanding challenge in the field. Accordingly, there remains a need of developing GLP-1 receptor agonists for an easily-administered prevention and/or treatment for cardiometabolic and associated diseases.

### SUMMARY

Disclosed are compounds that can be used as glucagon-like peptide-1 receptor (GLP-1R) agonists, compositions containing these compounds and methods for treating diseases and/or conditions mediated by GLP-1R.

In an aspect, the present disclosure provides a compound of Formula (I**):

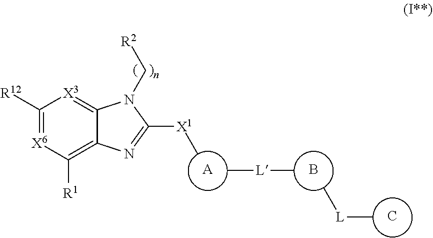

or a pharmaceutically acceptable salt thereof; wherein:
X³ is CR⁶ or N;
X⁶ is CR⁴ or N;
R¹ is —C₁₋₆ haloalkyl, halogen, —O—X⁴, or —NR⁸R⁹, or R¹ and R⁴, together with the atoms to which they are attached, combine to form a 5- or 6-membered heterocyclyl;
X⁴ is hydrogen, C₁₋₆ alkyl, C₁₋₆ heteroalkyl, C₁₋₆ haloalkyl, —(C₁₋₆ alkyl)-(C₃₋₁₀ cycloalkyl), or —(C₁₋₆ alkyl)-(3- to 8-membered heterocyclyl), 3- to 8-membered heterocyclyl, —(CH₂CH₂—O)₁₋₅—CH₃, —(CH₂—CH(—OCH₃)—CH₂—O)₁₋₅—CH₃, C₃₋₁₀ cycloalkyl, or C₆₋₁₀ aryl, wherein the alkyl, heteroalkyl, or haloalkyl group is optionally substituted with one or more deuterium, C₁₋₆ alkoxy, hydroxyl, —CN, or oxo, and the cycloalkyl, heterocyclyl, or aryl group is optionally substituted with one or more halogen, C₁₋₆ alkoxy, or —CN;
R⁶ is hydrogen, halogen, or —O—R⁷;
   wherein R⁷ and R², together with the atoms to which they are attached, combine to form a 6-membered heterocyclyl;
R⁸ and R⁹ each independently are selected from hydrogen, C₁₋₆ alkyl, or —S(O)₂—C₁₋₆ alkyl, or R⁸ and R⁹, together with the atoms to which they are attached, combine to form a 6-membered heterocycyl; wherein the C₁₋₆ alkyl is optionally substituted by one or more oxo;
n is 0, 1, 2, 3, 4, 5, or 6;
R² is:
   hydrogen;
   C₁₋₆ alkyl optionally substituted with deuterium;
   C₁₋₆ haloalkyl;
   —(O)—C₁₋₆ alkyl;
   —CN;
   a C₃₋₁₀ cycloalkyl optionally substituted with one or more —CN, C₁₋₆ haloalkyl, or C₁₋₆ alkyl optionally substituted with one or more —CN;
   a 4 or 5-membered heterocyclyl comprising at least one oxygen or at least one sulfur atom, wherein the 4- or 5-membered heterocyclyl is optionally substituted with one or more oxo,

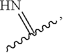

or C₁₋₆ alkyl;
a 5-membered heteroaryl, comprising 1 or 2 heteroatoms independently selected from N, and S, wherein at least one heteroatom of R⁵ is S; or
R² and R⁷, together with the atoms to which they are attached, combine to form a 5- or 6-membered heterocyclyl;
R⁴ is halogen, hydrogen, —C(O)OH, or —O—R⁸,
   wherein R⁸ and R¹, together with the atoms to which they are attached, combine to form a 6-membered heterocyclyl;

R¹² is hydrogen, —C(O)OH, —C(O)NR^{N12}R^{N12′}, —C(O)NR¹²S(O)₂R¹², —(C₂₋₆ alkynylene)-C(O)OH, —(C₁₋₆ alkylene)-C(O)OH, —NR^{N12}—(C₁₋₆ alkylene)-C(O)OH, 5-10 membered heteroaryl or 5- to 10-membered heterocyclyl optionally substituted with one or more oxo, C₁₋₆ alkyl or C₁₋₆ haloalkyl;
R^{N12} and R^{N12′} independently are H or C₁₋₆ alkyl;

X¹ is

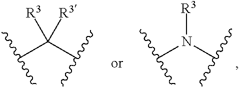

wherein R³ and R^{3′} independently are H, D or C₁₋₆ alkyl, wherein the C₁₋₆ alkyl is optionally substituted with deuterium;
Ring A is

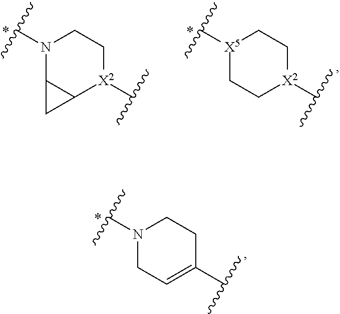

phenyl optionally substituted with one or more halo or C₁₋₆ alkyl, or 6-membered heteroaryl optionally substituted with one or more halo or C₁₋₆ alkyl;
wherein * indicates attachment to X¹,
   X⁵ is CR³ or N, and
   X² is CR³ or N;
L′ is a bond or —O—;
Ring B is a C₆₋₁₀ arylene, a 5-10 membered heteroarylene, or a 3-10 membered heterocycylene, wherein the C₆₋₁₀ arylene, 5-10 membered heteroarylene, or 3-10 membered heterocyclene is optionally substituted with one or more oxo, C₁₋₆ alkyl, C₁₋₆ alkoxy, or halogen;
L is a bond, *—(C₁₋₆ alkylene)-, *—NR^{L}—(C₁₋₆ alkyl), *—O—(C₁₋₆ alkyl)-, or *—(C₁₋₆ alkyl)-O—, wherein * indicates attachment to Ring B and the C₁₋₆ alkylene or C₁₋₆ alkyl is optionally substituted with deuterium;
   wherein R^{L} is H or C₁₋₆ alkyl; and
Ring C is:
a 6-membered aryl optionally substituted with one or more C₃₋₁₀ cycloalkyl, C₁₋₆ alkyl, C₁₋₆ haloalkyl, 3-10 membered heterocyclyl, halogen, C₁₋₆ alkoxy, C₁₋₆ haloalkoxy, —CN, C₃₋₁₀ cycloalkyl, or —C(O)NR′₂;
   wherein R′ is H or C₁₋₆ alkyl
a 6-membered heteroaryl comprising a nitrogen atom optionally substituted with one or more halogen, —CN, C₁₋₆ haloalkyl, —O—C₁₋₆ alkyl, C₃₋₁₀ cycloalkyl, —C(═O)—(C₃₋₁₀ cycloalkyl),

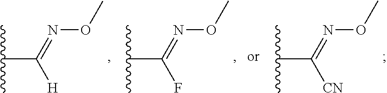

or
a bicylic 9- or 10-membered heteroaryl or heterocyclyl optionally substituted with one or more C₁₋₆ alkyl, halogen, —CN, or oxo.

In an aspect, the present disclosure provides a compound of Formula (I**-4):

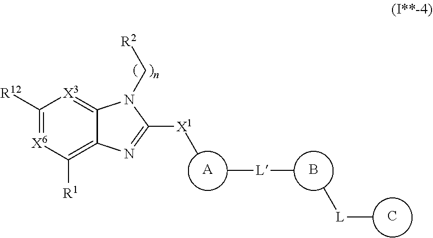

or a pharmaceutically acceptable salt thereof, wherein:
X³ is CR⁶ or N;
X⁶ is CR⁴ or N;
R¹ is —C₁₋₆ haloalkyl, halogen, —O—X⁴, or —NR⁸R⁹, or R¹ and R⁴, together with the atoms to which they are attached, combine to form a 6-membered heterocyclyl;
X⁴ is hydrogen, C₁₋₆ alkyl, C₁₋₆ heteroalkyl, C₁₋₆ haloalkyl, —(C₁₋₆ alkyl)-(C₃₋₁₀ cycloalkyl), or —(C₁₋₆ alkyl)-(3- to 8-membered heterocyclyl), 3- to 8-membered heterocyclyl, —(CH₂CH₂—O)₁₋₅—CH₃, or —(CH₂—CH(—OCH₃)—CH₂—O)₁₋₅—CH₃, wherein the alkyl, heteroalkyl, or haloalkyl group is optionally substituted with one or more deuterium, C₁₋₆ alkoxy, hydroxyl, —CN, or oxo, and the cycloalkyl or heterocyclyl group is optionally substituted with one or more halogen or —CN;
R⁶ is hydrogen, halogen, or —O—R⁷;
   wherein R⁷ and R², together with the atoms to which they are attached, combine to form a 6-membered heterocyclyl;
R⁸ and R⁹ each independently are selected from hydrogen, C₁₋₆ alkyl, or —S(O)₂—C₁₋₆ alkyl, or R⁸ and R⁹, together with the atoms to which they are attached, combine to form a 6-membered heterocycyl; wherein the C₁₋₆ alkyl is optionally substituted by one or more oxo;
n is 0, 1, 2, 3, 4, 5, or 6;
R² is:
   hydrogen;
   C₁₋₆ alkyl optionally substituted with deuterium;
   C₁₋₆ haloalkyl;
   —(O)—C₁₋₆ alkyl;
   —CN;
   a C₃₋₁₀ cycloalkyl optionally substituted with one or more —CN, C₁₋₆ haloalkyl, or C₁₋₆ alkyl optionally substituted with one or more —CN;
   a 4 or 5-membered heterocyclyl comprising at least one oxygen or at least one sulfur atom, wherein the 4- or 5-membered heterocyclyl is optionally substituted with one or more oxo,

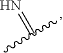

or C₁₋₆ alkyl;
a 5-membered heteroaryl, comprising 1 or 2 heteroatoms independently selected from N, and S, wherein at least one heteroatom of R⁵ is S; or
R² and R⁷, together with the atoms to which they are attached, combine to form a 5- or 6-membered heterocyclyl;
R⁴ is hydrogen, —C(O)OH, or —O—R⁸,
   wherein R⁸ and R¹, together with the atoms to which they are attached, combine to form a 6-membered heterocyclyl;
R¹² is hydrogen, —C(O)OH or 5-10 membered heteroaryl optionally substituted with one or more oxo, C₁₋₆ alkyl or C₁₋₆ haloalkyl;

X¹ is

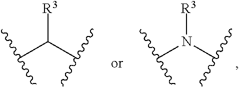

wherein R³ is H or C₁₋₆ alkyl;
Ring A is

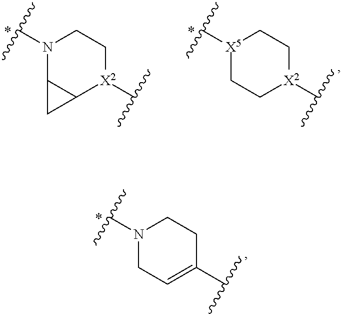

phenyl optionally substituted with one or more halo or C₁₋₆ alkyl, or 6-membered heteroaryl optionally substituted with one or more halo or C₁₋₆ alkyl;
wherein * indicates attachment to X¹,
   X⁵ is CR³ or N, and
   X² is CR³ or N;
L′ is a bond or —O—;
Ring B is a C₆₋₁₀ arylene, a 5-10 membered heteroarylene, or a 3-10 membered heterocycylene, wherein the C₆₋₁₀ arylene, 5-10 membered heteroarylene, or 3-10 membered heterocyclene is optionally substituted with one or more oxo, C₁₋₆ alkyl, C₁₋₆ alkoxy, or halogen;
L is a bond, *—(C₁₋₆ alkylene)-, *—NR^{L}—(C₁₋₆ alkyl), *—O—(C₁₋₆ alkyl)-, or *—(C₁₋₆ alkyl)-O—, wherein * indicates attachment to Ring B and the C₁₋₆ alkylene or C₁₋₆ alkyl is optionally substituted with deuterium;
   wherein R^{L} is H or C₁₋₆ alkyl; and
Ring C is:
a 6-membered aryl optionally substituted with one or more C₁₋₆ alkyl, C₁₋₆ haloalkyl, 3-10 membered heterocyclyl, halogen, C₁₋₆ alkoxy, C₁₋₆ haloalkoxy, —CN, C₃₋₁₀ cycloalkyl, or —C(O)NR′₂;
   wherein R′ is H or C₁₋₆ alkyl
a 6-membered heteroaryl comprising a nitrogen atom optionally substituted with one or more halogen, —CN, C₁₋₆ haloalkyl, —O—C₁₋₆ alkyl, C₃₋₁₀ cycloalkyl, —C(═O)—(C₃₋₁₀ cycloalkyl),

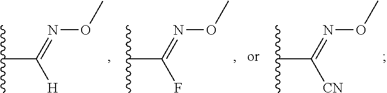

or
   a bicylic 9- or 10-membered heteroaryl optionally substituted with one or more C₁₋₆ alkyl, halogen, or oxo.

In some embodiments, the compound of Formula (I**) is of Formula I*:

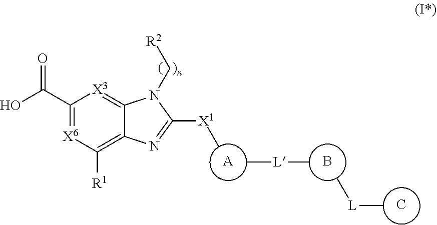

or a pharmaceutically acceptable salt thereof; wherein:
X³ is CR⁶ or N;
X⁶ is CR⁴ or N;
R¹ is —C₁₋₆ haloalkyl, —O—X⁴, or —NR⁸R⁹, or R¹ and R⁴, together with the atoms to which they are attached, combine to form a 6-membered heterocyclyl;
X⁴ is hydrogen, C₁₋₆ alkyl, C₁₋₆ heteroalkyl, C₁₋₆ haloalkyl, —(C₁₋₆ alkyl)-(C₃₋₁₀ cycloalkyl), or —(C₁₋₆ alkyl)-(3- to 8-membered heterocyclyl), 3- to 8-membered heterocyclyl, —(CH₂CH₂—O)₁₋₅—CH₃, or —(CH₂—CH(—OCH₃)—CH₂—O)₁₋₅—CH₃, wherein the alkyl, heteroalkyl, or haloalkyl group is optionally substituted with one or more deuterium, hydroxyl, —CN, or oxo, and the cycloalkyl or heterocyclyl group is optionally substituted with one or more halogen or —CN;
R⁶ is hydrogen, halogen, or —O—R⁷;
   wherein R⁷ and R², together with the atoms to which they are attached, combine to form a 6-membered heterocyclyl;
R⁸ and R⁹ each independently are selected from hydrogen, C₁₋₆ alkyl, or —S(O)₂—C₁₋₆ alkyl, or R⁸ and R⁹, together with the atoms to which they are attached, combine to form a 6-membered heterocycyl; wherein the C₁₋₆ alkyl is optionally substituted by one or more oxo;
n is 0, 1, 2, 3, 4, 5, or 6;
R² is:
hydrogen;
C₁₋₆ alkyl;
C₁₋₆ haloalkyl;
—(O)—C₁₋₆ alkyl;
a C₃₋₁₀ cycloalkyl optionally substituted with one or more —CN, C₁₋₆ haloalkyl, or C₁₋₆ alkyl optionally substituted with one or more —CN;
a 4 or 5-membered heterocyclyl comprising at least one oxygen or at least one sulfur atom, wherein the 4- or 5-membered heterocyclyl is optionally substituted with one or more oxo,

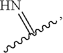

or C₁₋₆ alkyl;
a 5-membered heteroaryl, comprising 1 or 2 heteroatoms independently selected from N, and S, wherein at least one heteroatom of R⁵ is S; or
R² and R⁷, together with the atoms to which they are attached, combine to form a 5- or 6-membered heterocyclyl;
R⁴ is hydrogen or —O—R⁸,
   wherein R⁸ and R¹, together with the atoms to which they are attached, combine to form a 6-membered heterocyclyl;
X¹ is

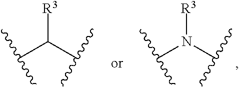

wherein R³ is H or C₁₋₆ alkyl;
Ring A is

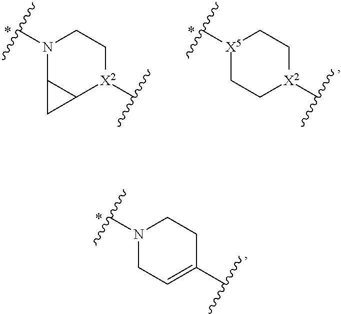

phenyl optionally substituted with one or more halo or C₁₋₆ alkyl, or 6-membered heteroaryl optionally substituted with one or more halo or C₁₋₆ alkyl;
wherein * indicates attachment to X¹,
   X⁵ is CR³ or N, and
   X² is CR³ or N;
L′ is a bond or —O—;
Ring B is a C₆₋₁₀ arylene, a 6-10 membered heteroarylene, or a 3-10 membered heterocycylene, wherein the C₆₋₁₀ arylene, 6-10 heteroarylene, or 3-10 membered heterocyclene is optionally substituted with one or more C₁₋₆ alkyl, C₁₋₆ alkoxy, or halogen;
L is a bond, *—CH₂—, *—O—(C₁₋₆ alkyl)-, or *—(C₁₋₆ alkyl)-O—, wherein * indicates attachment to Ring B and the C₁₋₆ alkyl is optionally substituted with deuterium; and
Ring C is:
a 6-membered aryl optionally substituted with one or more C₁₋₆ alkyl, 3-10 membered heterocyclyl, halogen, —OCH₃, —CN, or C₃₋₁₀ cycloalkyl;
a 6-membered heteroaryl comprising a nitrogen atom optionally substituted with one or more halogen, —CN, —O—C₁₋₆ alkyl, C₃₋₁₀ cycloalkyl, —C(═O)—(C₃₋₁₀ cycloalkyl),

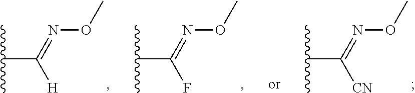

or
   a bicylic 9- or 10-membered heteroaryl comprising two nitrogen atoms optionally substituted with one or more C₁₋₆ alkyl, or oxo.

In an aspect, the present disclosure provides a compound of Formula I″:

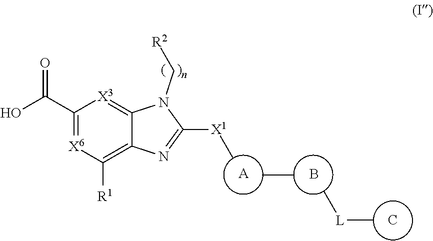

or a pharmaceutically acceptable salt thereof; wherein:
X³ is CR⁶ or N;
X⁶ is CR⁴ or N;
R¹ is —C₁₋₆ haloalkyl, —O—X⁴, or —NR⁸R⁹, or R¹ and R⁴, together with the atoms to which they are attached, combine to form a 6-membered heterocyclyl;
wherein X⁴ is hydrogen, C₁₋₆ alkyl, C₁₋₆ heteroalkyl, C₁₋₆ haloalkyl, —(C₁₋₆ alkyl)-(C₃₋₁₀ cycloalkyl), or —(C₁₋₆ alkyl)-(3- to 8-membered heterocyclyl), 3- to 8-membered heterocyclyl, —(CH₂CH₂—O)₁₋₅—CH₃, or —(CH₂—CH(—OCH₃)—CH₂—O)₁₋₅—CH₃, wherein the alkyl, heteroalkyl, or haloalkyl group is optionally substituted with one or more deuterium, hydroxyl, —CN, or oxo, and the cycloalkyl or heterocyclyl group is optionally substituted with one or more halogen or —CN;
R⁶ is hydrogen, halogen, or —O—R⁷;
   wherein R⁷ and R², together with the atoms to which they are attached, combine to form a 5- or 6-membered heterocyclyl;
R⁸ and R⁹ each independently are selected from hydrogen, C₁₋₆ alkyl, or —S(O)₂—C₁₋₆ alkyl, or R⁸ and R⁹, together with the atoms to which they are attached, combine to form a 6-membered heterocycyl; wherein the C₁₋₆ alkyl is optionally substituted by one or more oxo;
n is 0, 1, 2, 3, 4, 5, or 6;
R² is:
hydrogen;
C₁₋₆ alkyl;
—(O)—C₁₋₆ alkyl;
a C₃₋₁₀ cycloalkyl optionally substituted with one or more —CN, C₁₋₆ haloalkyl, or C₁₋₆ alkyl optionally substituted with one or more —CN;
a 4 or 5-membered heterocyclyl comprising at least one oxygen or at least one sulfur atom, wherein the 4- or 5-membered heterocyclyl is optionally substituted with one or more oxo,

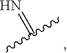 [SMILES: *=N]

or C₁₋₆ alkyl;
a 5-membered heteroaryl, comprising 1 or 2 heteroatoms independently selected from N, and S, wherein at least one heteroatom of R⁵ is S; or
R² and R⁷, together with the atoms to which they are attached, combine to form a C₂₋₉ heterocyclyl;
R⁴ is hydrogen or —O—R⁸,
   wherein R⁸ and R¹, together with the atoms to which they are attached, combine to form a 6-membered heterocyclyl;
X¹ is

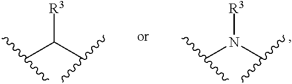

wherein R³ is H or C₁₋₆ alkyl;
Ring A is

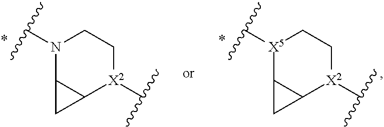

wherein * indicates attachment to X¹,
   X⁵ is CH or N, and
   X² is CH or N;
Ring B is a 6-10 membered heteroarylene, or a 3-10 membered heterocycylene optionally substituted with one or more C₁₋₆ alkyl;
L is a bond or *—O—(C₁₋₆ alkyl)-, wherein * indicates attachment to Ring B; and
Ring C is:
a 6-membered aryl optionally substituted with one or more halogen, —OCH₃, —CN, or C₃₋₁₀ cycloalkyl;
a 6-membered heteroaryl comprising a nitrogen atom optionally substituted with one or more halogen, —CN, —O—C₁₋₆ alkyl, C₃₋₁₀ cycloalkyl, —C(═O)—(C₃₋₁₀ cycloalkyl),

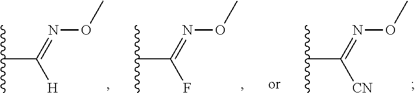

or
   a bicylic 9- or 10-membered heteroaryl comprising two nitrogen atoms optionally substituted with one or more C₁₋₆ alkyl, or oxo.

In an aspect, the present disclosure provides a compound of Formula (I′″*):

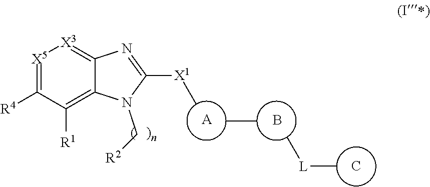

or a pharmaceutically acceptable salt thereof; wherein:
X³ is CR^{X3} or N;
   wherein R^{X3} is H, halogen, C₁₋₆ alkyl, C₁₋₆ haloalkoxy, —O—(CH₂CH₂—O)₁₋₅—CH₃, or C₁₋₆ alkoxy;
X^{5′} is CR^{5′}
R¹ is —C₁₋₆ haloalkyl or —O—X⁴, or R¹ and R⁴, together with the atoms to which they are attached, combine to form a 6-membered heterocyclyl;
wherein X⁴ is hydrogen, C₁₋₆ alkyl, C₁₋₆ heteroalkyl, C₁₋₆ haloalkyl, —(C₁₋₆ alkyl)-(C₃₋₁₀ cycloalkyl), or —(C₁₋₆ alkyl)-(3- to 8-membered heterocyclyl), 3- to 8-membered heterocyclyl, —(CH₂CH₂—O)₁₋₅—CH₃, or —(CH₂—CH(—OCH₃)—CH₂—O)₁₋₅—CH₃, wherein the alkyl, heteroalkyl, or haloalkyl group is optionally substituted with one or more deuterium, halogen, or —CN, and the cycloalkyl or heterocyclyl group is optionally substituted with one or more halogen or —CN;
n is 0, 1, 2, 3, 4, 5, or 6;
R² is:
   hydrogen;
   halogen;
   branched C₃₋₆ alkyl;
   —(O)—C₁₋₆ alkyl;
   C₃₋₁₀ cycloalkyl optionally substituted with one or more —CN, C₁₋₆ haloalkyl, or C₁₋₆ alkyl optionally substituted with one or more —CN;
   4 or 5-membered heterocyclyl comprising at least one oxygen or at least one sulfur atom, wherein the 4- or 5-membered heterocyclyl is optionally substituted with one or more oxo,

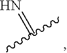 [SMILES: *=N]

or C₁₋₆ alkyl; or
5-membered heteroaryl, comprising 1 or 2 heteroatoms independently selected from N, and S, wherein at least one heteroatom of R⁵ is S; or
R² and R¹, together with the atoms to which they are attached, combine to form a 6-membered heterocyclyl;
R^{5′} is hydrogen or —C(O)—OH;
R⁴ is hydrogen, halogen, —C(O)—OH, or —O—R⁸,
   wherein R⁸ and R¹, together with the atoms to which they are attached, combine to form a 6-membered heterocyclyl; X¹ is

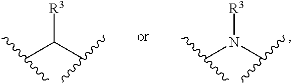

wherein R³ is H or C₁₋₆ alkyl;

### Ring A is

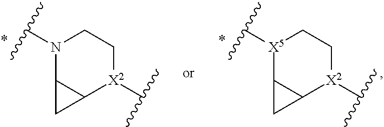 [0166] wherein * indicates attachment to X¹,
wherein X⁵ is CH or N, and
   X² is CH or N;
Ring B is a 6-10 membered heteroarylene, or a 3-10 membered heterocycylene optionally substituted with one or more C₁₋₆ alkyl;

L is a bond or *—O—(C₁₋₆ alkyl)-, wherein * indicates attachment to Ring B; Ring C is:
a 6-membered aryl optionally substituted with one or more halogen, —OCH₃, —CN, or C₃₋₁₀ cycloalkyl;
a 6-membered heteroaryl comprising a nitrogen atom optionally substituted with one or more halogen, —CN, —O—C₁₋₆ alkyl, C₃₋₁₀ cycloalkyl, —C(═O)—(C₃₋₁₀ cycloalkyl),

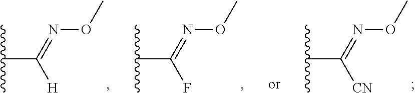

or
a bicylic 9- or 10-membered heteroaryl comprising two nitrogen atoms optionally substituted with one or more C₁₋₆ alkyl, or oxo.

In an aspect, the present disclosure provides a compound of Formula (I′″)

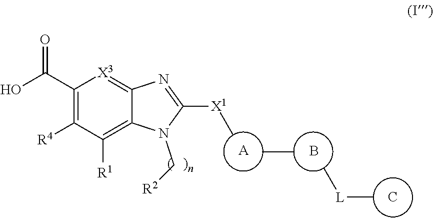

or a pharmaceutically acceptable salt thereof, wherein:
X³ is CR^{X3} or N;
   wherein R^{X3} is H, halogen, C₁₋₆ alkyl, C₁₋₆ haloalkoxy, or C₁₋₆ alkoxy;
R¹ is —C₁₋₆ haloalkyl or —O—X⁴, or R¹ and R⁴, together with the atoms to which they are attached, combine to form a 6-membered heterocyclyl;
wherein X⁴ is hydrogen, C₁₋₆ alkyl, C₁₋₆ heteroalkyl, C₁₋₆ haloalkyl, —(C₁₋₆ alkyl)-(C₃₋₁₀ cycloalkyl), or —(C₁₋₆ alkyl)-(3- to 8-membered heterocyclyl), 3- to 8-membered heterocyclyl, —(CH₂CH₂—O)₁₋₅—CH₃, or —(CH₂—CH(—OCH₃)—CH₂—O)₁₋₅—CH₃, wherein the alkyl, heteroalkyl, or haloalkyl group is optionally substituted with one or more deuterium, —CN, and the cycloalkyl or heterocyclyl group is optionally substituted with one or more halogen or —CN;
n is 0, 1, 2, 3, 4, 5, or 6;
R² is:
   hydrogen;
   halogen;
   C₃₋₆ alkyl;
   —(O)—C₁₋₆ alkyl;
   a C₃₋₁₀ cycloalkyl optionally substituted with one or more —CN, C₁₋₆ haloalkyl, or C₁₋₆ alkyl optionally substituted with one or more —CN;
   a 4 or 5-membered heterocyclyl comprising at least one oxygen or at least one sulfur atom, wherein the 4- or 5-membered heterocyclyl is optionally substituted with one or more oxo,

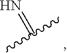 [SMILES: *=N]

or C₁₋₆ alkyl;
a 5-membered heteroaryl, comprising 1 or 2 heteroatoms independently selected from N, and S, wherein at least one heteroatom of R⁵ is S; or
R² and R¹, together with the atoms to which they are attached, combine to form a 6-membered heterocyclyl;
R⁴ is hydrogen or —O—R⁸,
   wherein R⁸ and R¹, together with the atoms to which they are attached, combine to form a 6-membered heterocyclyl;
X¹ is

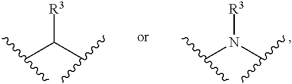

wherein R³ is H or C₁₋₆ alkyl;

### Ring A is

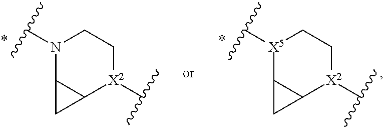 [0194] wherein * indicates attachment to X¹,
wherein X⁵ is CH or N, and
   X² is CH or N;
Ring B is a 6-10 membered heteroarylene, or a 3-10 membered heterocycylene optionally substituted with one or more C₁₋₆ alkyl;
L is a bond or *—O—(C₁₋₆ alkyl)-, wherein * indicates attachment to Ring B;
Ring C is:
a 6-membered aryl optionally substituted with one or more halogen, —OCH₃, —CN, or C₃₋₁₀ cycloalkyl;
a 6-membered heteroaryl comprising a nitrogen atom optionally substituted with one or more halogen, —CN, —O—C₁₋₆ alkyl, C₃₋₁₀ cycloalkyl, —C(═O)—(C₃₋₁₀ cycloalkyl),

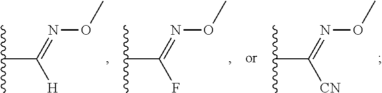

a bicylic 9- or 10-membered heteroaryl comprising two nitrogen atoms optionally substituted with one or more C₁₋₆ alkyl, or oxo.

In some embodiments of Formula (I″″), or a pharmaceutically acceptable salt thereof,
X³ is CH or N;
R¹ is —C₁₋₆ haloalkyl or —O—X⁴, or R¹ and R⁴, together with the atoms to which they are attached, combine to form a 6-membered heterocyclyl;
wherein X⁴ is hydrogen, C₁₋₆ alkyl, C₁₋₆ heteroalkyl, C₁₋₆ haloalkyl, —(C₁₋₆ alkyl)-(C₃₋₁₀ cycloalkyl), or —(C₁₋₆ alkyl)-(3- to 8-membered heterocyclyl), 3- to 8-membered heterocyclyl, —(CH₂CH₂—O)₁₋₅—CH₃, or —(CH₂—CH(—OCH₃)—CH₂—O)₁₋₅—CH₃, wherein the alkyl, heteroalkyl, or haloalkyl group is optionally substituted with one or more deuterium, —CN, and the cycloalkyl or heterocyclyl group is optionally substituted with one or more halogen or —CN;
n is 0, 1, 2, 3, 4, 5, or 6;
R² is:
hydrogen;
C₃₋₆ alkyl;
—(O)—C₁₋₆ alkyl;
a C₃₋₁₀ cycloalkyl optionally substituted with one or more —CN, C₁₋₆ haloalkyl, or C₁₋₆ alkyl optionally substituted with one or more —CN;
a 4 or 5-membered heterocyclyl comprising at least one oxygen or at least one sulfur atom, wherein the 4- or 5-membered heterocyclyl is optionally substituted with one or more oxo,

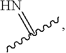 [SMILES: *=N]

or C₁₋₆ alkyl;
a 5-membered heteroaryl, comprising 1 or 2 heteroatoms independently selected from N, and S, wherein at least one heteroatom of R⁵ is S; or
R² and R¹, together with the atoms to which they are attached, combine to form a 6-membered heterocyclyl;
R⁴ is hydrogen or —O—R⁸,
   wherein R⁸ and R¹, together with the atoms to which they are attached, combine to form a 6-membered heterocyclyl;
X¹ is

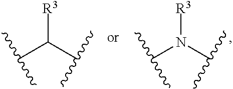

wherein R³ is H or C₁₋₆ alkyl;

### Ring A is

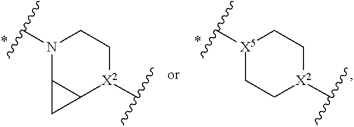 [0220] wherein * indicates attachment to X¹,
wherein X⁵ is CH or N, and
   X² is CH or N;
Ring B is a 6-10 membered heteroarylene, or a 3-10 membered heterocycylene optionally substituted with one or more C₁₋₆ alkyl;
L is a bond or *—O—(C₁₋₆ alkyl)-, wherein * indicates attachment to Ring B;
Ring C is:
a 6-membered aryl optionally substituted with one or more halogen, —OCH₃, —CN, or C₃₋₁₀ cycloalkyl;
a 6-membered heteroaryl comprising a nitrogen atom optionally substituted with one or more halogen, —CN, —O—C₁₋₆ alkyl, C₃₋₁₀ cycloalkyl, —C(═O)—(C₃₋₁₀ cycloalkyl),

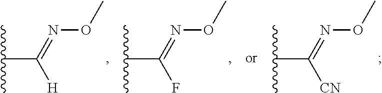

a bicylic 9- or 10-membered heteroaryl comprising two nitrogen atoms optionally substituted with one or more C₁₋₆ alkyl, or oxo.

In some embodiments, the compound of Formula (I*) is of Formula (I′):

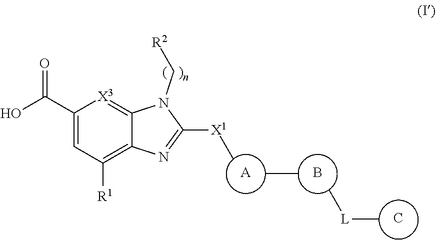

or a pharmaceutically acceptable salt thereof; wherein:
X³ is CH or N;
R¹ is —C₁₋₆ haloalkyl or —O—X⁴;
wherein X⁴ is hydrogen, C₁₋₆ alkyl, C₁₋₆ heteroalkyl, C₁₋₆ haloalkyl, —(C₁₋₆ alkyl)-(C₃₋₁₀ cycloalkyl), —(C₁₋₆ alkyl)-(3- to 8-membered heterocyclyl), 3- to 8-membered heterocyclyl, —(CH₂CH₂—O)₁₋₅—CH₃, or —(CH₂—CH(—OCH₃)—CH₂—O)₁₋₅—CH₃, wherein the alkyl, heteroalkyl, or haloalkyl group is optionally substituted with one or more —CN, and the cycloalkyl or heterocyclyl group is optionally substituted with one or more halogen or —CN;
n is 0, 1, 2, 3, 4, 5, or 6;
R² is hydrogen;
—(O)—C₁₋₆ alkyl;
a C₃₋₁₀ cycloalkyl optionally substituted with one or more —CN, C₁₋₆ haloalkyl, or C₁₋₆ alkyl optionally substituted with one or more —CN;
a 4 or 5-membered heterocyclyl comprising at least one oxygen or at least one sulfur atom, wherein the 4- or 5-membered heterocyclyl is optionally substituted with one or more oxo,

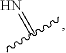 [SMILES: *=N]

or C₁₋₆ alkyl; or
a 5-membered heteroaryl, comprising 1 or 2 heteroatoms independently selected from N, and S, wherein at least one heteroatom of R⁵ is S;
X¹ is

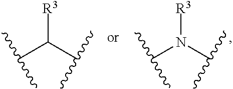

wherein R³ is H or C₁₋₆ alkyl;

### Ring A is

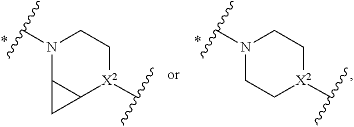 [0241] wherein * indicates attachment to X¹, and
wherein X² is CH or N;
Ring B is a 6-10 membered heteroarylene, or a 3-10 membered heterocycylene optionally substituted with one or more C₁₋₆ alkyl;
L is a bond or *—O—(C₁₋₆ alkyl)-, wherein * indicates attachment to Ring B; Ring C is:
a 6-membered aryl optionally substituted with one or more halogen, —OCH₃, —CN, or C₃₋₁₀ cycloalkyl;
a 6-membered heteroaryl comprising a nitrogen atom optionally substituted with one or more halogen, —CN, —O—C₁₋₆ alkyl, C₃₋₁₀ cycloalkyl, —C(═O)—(C₃₋₁₀ cycloalkyl),

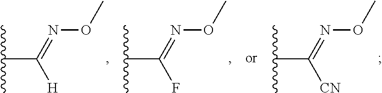

a bicylic 9- or 10-membered heteroaryl comprising two nitrogen atoms optionally substituted with one or more C₁₋₆ alkyl, or oxo.

In some embodiments, the compound of Formula (I′″) is of Formula (I):

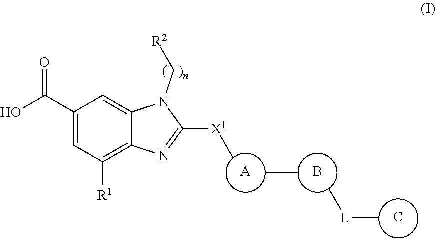

or a pharmaceutically acceptable salt thereof; wherein
R¹ is —C₁₋₆ haloalkyl or —O—X⁴;
wherein X⁴ is C₁₋₆ alkyl, C₁₋₆ heteroalkyl, C₁₋₆ haloalkyl, —(C₁₋₆ alkyl)-(C₃₋₁₀ cycloalkyl), or —(C₁₋₆ alkyl)-(3- to 8-membered heterocyclyl), —(—CH₂CH₂—O)₁₋₅—CH₃, or —(CH₂—CH(—OCH₃)—CH₂—O)₁₋₅—CH₃, wherein the alkyl, heteroalkyl, or haloalkyl group is optionally substituted with one or more —CN, and the cycloalkyl or heterocyclyl group is optionally substituted with one or more halogen or —CN;
n is 0, 1, 2, 3, 4, 5, or 6;
R² is:
hydrogen;
—(O)—C₁₋₆ alkyl;
a C₃₋₁₀ cycloalkyl optionally substituted with one or more —CN, C₁₋₆ haloalkyl, or C₁₋₆ alkyl optionally substituted with one or more —CN;
a 4 or 5-membered heterocyclyl comprising at least one oxygen or at least one sulfur atom, wherein the 4- or 5-membered heterocyclyl is optionally substituted with one or more oxo,

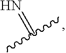 [SMILES: *=N]

or C₁₋₆ alkyl; or
a 5-membered heteroaryl, comprising 1 or 2 heteroatoms independently selected from N, and S, wherein at least one heteroatom of R⁵ is S;

X¹ is

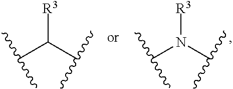

wherein R³ is H or C₁₋₆ alkyl;

### Ring A is

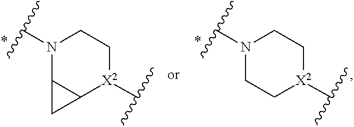 [0261] wherein * indicates attachment to X¹, and
wherein X² is CH or N;
Ring B is:
a 6-membered heteroarylene comprising nitrogen;
a 9-membered heterocycylene comprising two oxygen atoms optionally substituted with one or more C₁₋₆ alkyl; or
a 10-membered heterocycylene comprising two oxygen atoms;
L is a bond or *—O—(C₁₋₆ alkyl)-, wherein * indicates attachment to Ring B;
Ring C is:
a 6-membered aryl optionally substituted with one or more halogen, —OCH₃, —CN, or C₃₋₁₀ cycloalkyl;
a 6-membered heteroaryl comprising a nitrogen atom optionally substituted with one or more halogen, —CN, —O—C₁₋₆ alkyl, C₃₋₁₀ cycloalkyl, —C(═O)—(C₃₋₁₀ cycloalkyl),

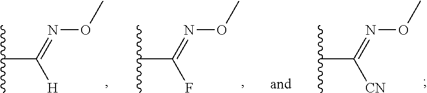

a bicylic 9- or 10-membered heteroaryl comprising two nitrogen atoms optionally substituted with one or more C₁₋₆ alkyl, or oxo.

In an aspect, the present disclosure provides a compound of Formula (P01):

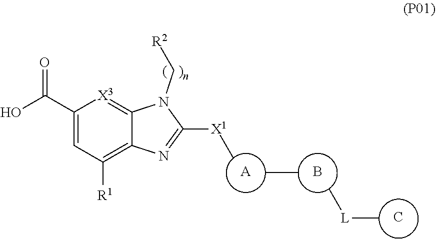

or a pharmaceutically acceptable salt thereof; wherein:
X³ is CH or N;
R¹ is —C₁₋₆ haloalkyl or —O—X⁴;
wherein X⁴ is C₁₋₆ alkyl, C₁₋₆ heteroalkyl, C₁₋₆ haloalkyl, —(C₁₋₆ alkyl)-(C₃₋₁₀ cycloalkyl), or —(C₁₋₆ alkyl)-(3- to 8-membered heterocyclyl), 3- to 8-membered heterocyclyl, —(CH₂CH₂—O)₁₋₅—CH₃, or —(CH₂—CH(—OCH₃)—CH₂—O)₁₋₅—CH₃, wherein the alkyl, heteroalkyl, or haloalkyl group is optionally substituted with one or more —CN, and the cycloalkyl or heterocyclyl group is optionally substituted with one or more halogen or —CN;
n is 0, 1, 2, 3, 4, 5, or 6;
R² is:
hydrogen;
—(O)—C₁₋₆ alkyl;
a C₃₋₁₀ cycloalkyl optionally substituted with one or more —CN, C₁₋₆ haloalkyl, or C₁₋₆ alkyl optionally substituted with one or more —CN;
a 4 or 5-membered heterocyclyl comprising at least one oxygen or at least one sulfur atom, wherein the 4- or 5-membered heterocyclyl is optionally substituted with one or more oxo,

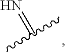 [SMILES: *=N]

or C₁₋₆ alkyl; or
a 5-membered heteroaryl, comprising 1 or 2 heteroatoms independently selected from N, and S, wherein at least one heteroatom of R⁵ is S;
X¹ is

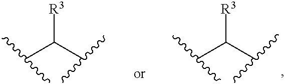

wherein R³ is H or C₁₋₆ alkyl;

### Ring A is

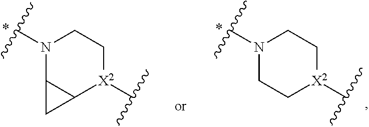 [0285] wherein * indicates attachment to X¹, and
wherein X² is CH or N;
Ring B is a 6-10 membered heteroarylene, or a 3-10 membered heterocycylene optionally substituted with one or more C₁₋₆ alkyl;
L is a bond or *—O—(C₁₋₆ alkyl)-, wherein * indicates attachment to Ring B;
Ring C is:
a 6-membered aryl optionally substituted with one or more halogen, —OCH₃, —CN, or C₃₋₁₀ cycloalkyl;
a 6-membered heteroaryl comprising a nitrogen atom optionally substituted with one or more halogen, —CN, —O—C₁₋₆ alkyl, C₃₋₁₀ cycloalkyl, —C(═O)—(C₃₋₁₀ cycloalkyl),

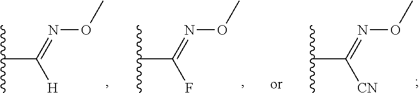

a bicylic 9- or 10-membered heteroaryl comprising two nitrogen atoms optionally substituted with one or more C₁₋₆ alkyl, or oxo.

In some embodiments, the compound of Formula (I*) is of Formula (IA):

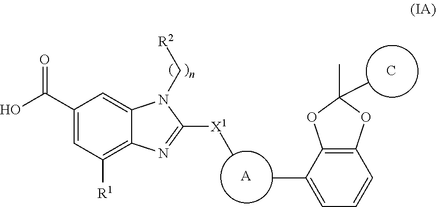

or a pharmaceutically acceptable salt thereof.

In some embodiments, the compound of Formula (I*) is of Formula (IB)

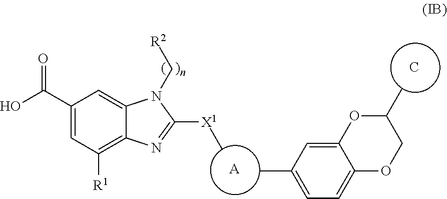

or a pharmaceutically acceptable salt thereof.

In some embodiments, the compound of Formula (I*) is of Formula (IC)

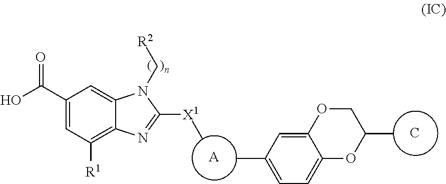

or a pharmaceutically acceptable salt thereof.

In some embodiments, the compound of Formula (I*) is of Formula (ID):

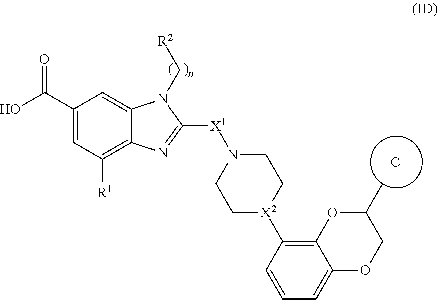

or a pharmaceutically acceptable salt thereof.

In some embodiments, the compound of Formula (I*), is of Formula (IE):

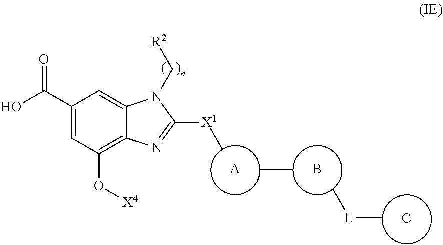

or a pharmaceutically acceptable salt thereof.

In some embodiments, the compound of Formula (I*) is of Formula (IF):

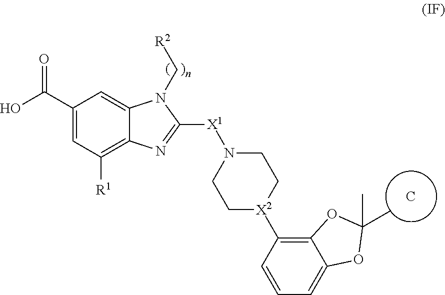

or a pharmaceutically acceptable salt thereof.

In some embodiments, the compound of Formula (I*) is of Formula (IG)

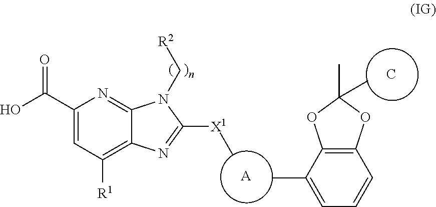

or a pharmaceutically acceptable salt thereof.

In some embodiments, the compound of Formula (I*) is of Formula (IH),

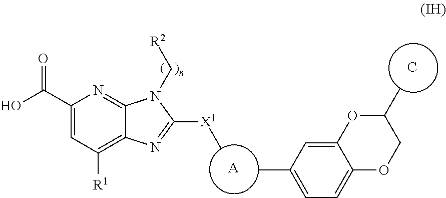

or a pharmaceutically acceptable salt thereof.

In some embodiments, the compound of Formula (I*) is of Formula (II)

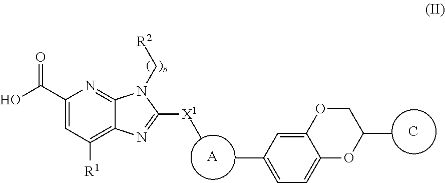

or a pharmaceutically acceptable salt thereof.

In some embodiments, the compound of Formula (I*) is of Formula (IJ):

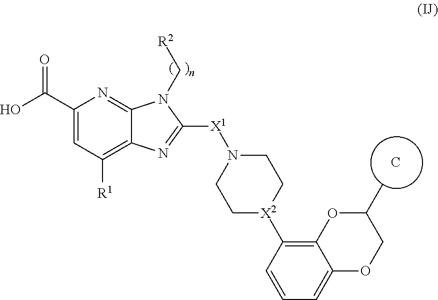

or a pharmaceutically acceptable salt thereof.

In some embodiments, the compound of Formula (I*) is of Formula (IK):

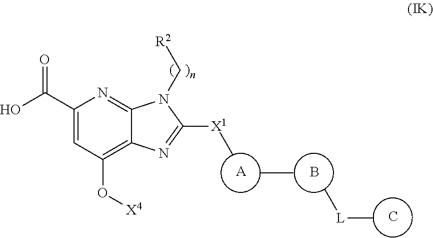

or a pharmaceutically acceptable salt thereof.

In some embodiments, the compound of Formula (I*) is of Formula (IL):

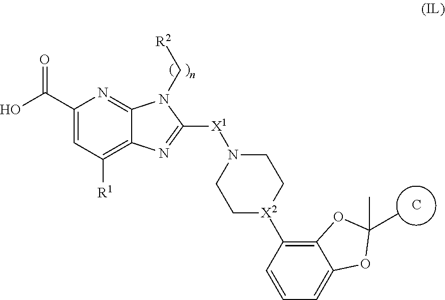

or a pharmaceutically acceptable salt thereof.

In some embodiments, the compound of Formula (I*) is of Formula (IM):

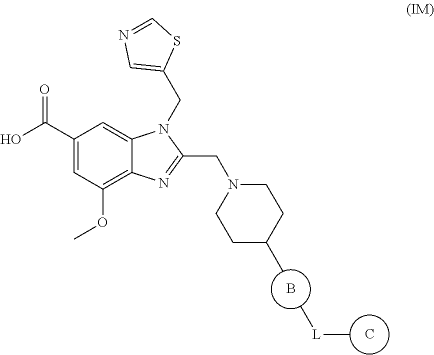

or a pharmaceutically acceptable salt thereof.

In some embodiments, the compound of Formula (I*) is of Formula (IN):

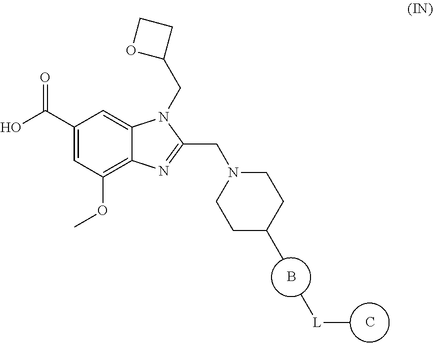

or a pharmaceutically acceptable salt thereof.

In some embodiments, the compound of Formula (I*) is of Formula (IO):

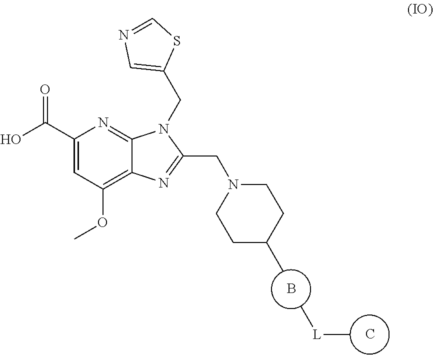

or a pharmaceutically acceptable salt thereof.

In some embodiments, the compound of Formula (I*) is of Formula (IP):

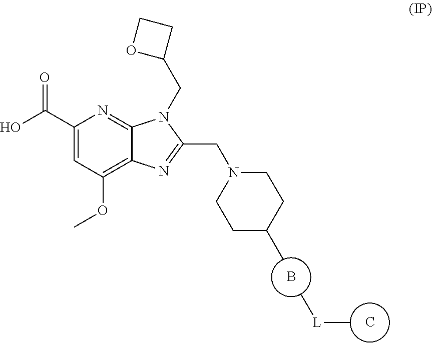

or a pharmaceutically acceptable salt thereof.

In some embodiments, the compound of Formula (I*) is of Formula (IQ)

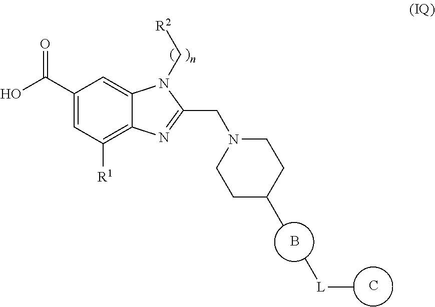

or a pharmaceutically acceptable salt thereof;
wherein R¹, R², n, Ring B, L, and Ring C are as defined for Formula (I*).

In some embodiments, the compound of Formula (I*) is of Formula (IR)

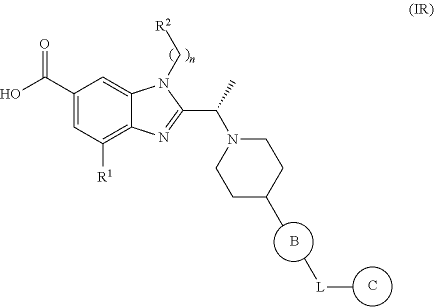

or a pharmaceutically acceptable salt thereof;
wherein R¹, R², n, Ring B, L, and Ring C are as defined for Formula (I*).

In some embodiments, the compound of Formula (I*) is of Formula (IS)

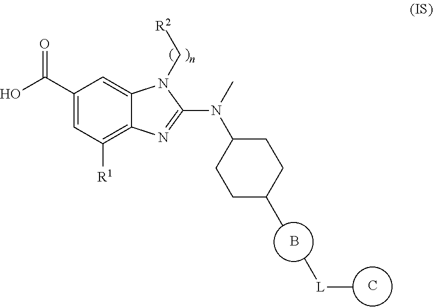

Wherein R¹, R², n, Ring B, L, and Ring C are as defined for Formula (I*).

In some embodiments, the compound of Formula (I*) is of Formula (IT)

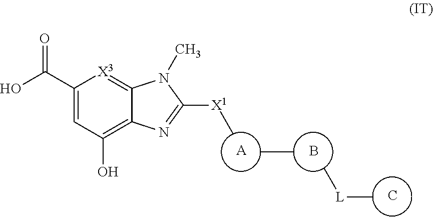

or a pharmaceutically acceptable salt thereof;
wherein X¹, X³, Ring A, Ring B, L, and Ring C are as defined for Formula (I*).

In some embodiments, the compound of Formula (I*) is of Formula (IU)

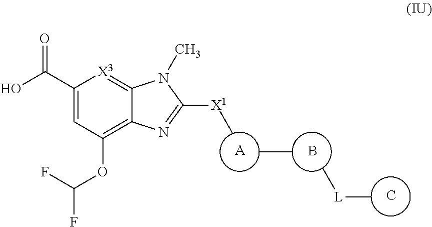

or a pharmaceutically acceptable salt thereof;
wherein X³, X¹, Ring A, Ring B, L, and Ring C are as defined for Formula (I*).

In some embodiments, the compound of Formula (I*) is of Formula (IX)

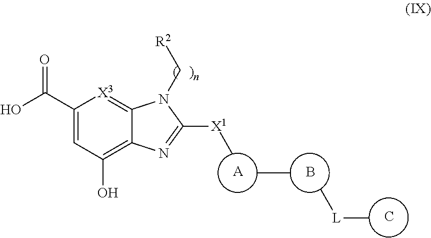

or a pharmaceutically acceptable salt thereof;
wherein X³, n, R², X¹, Ring A, Ring B, L, and Ring C are as defined for Formula (I*).

In some embodiments, the compound of Formula (I*) is of Formula (IY)

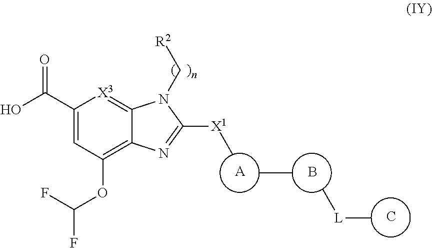

or a pharmaceutically acceptable salt thereof;
wherein X³, n, R², X¹, Ring A, Ring B, L, and Ring C are as defined for Formula (I*).

In some embodiments, the compound of Formula (I*) is of Formula (IZ)

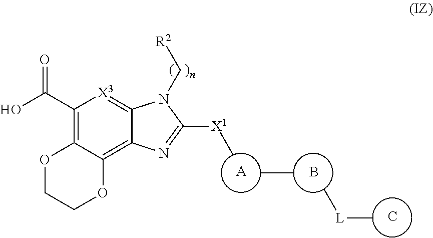

or a pharmaceutically acceptable salt thereof;
wherein X³, n, R², X¹, Ring A, Ring B, L, and Ring C are as defined for Formula (I*).

In some embodiments, the compound of Formula (I′″) is of Formula (IAA)

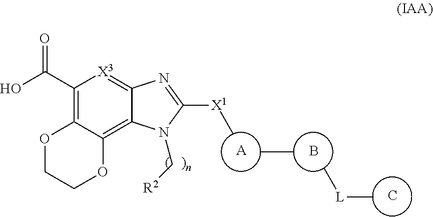

or a pharmaceutically acceptable salt thereof;
wherein X³, n, R², X¹, Ring A, Ring B, L, and Ring C are as defined for Formula (I*).

In some embodiments, the compound of Formula (I*) is of Formula (IAB):

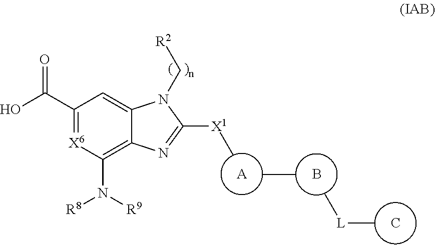

wherein X⁶, R⁸, R⁹, n, R², X¹, Ring A, Ring B, L, and Ring C are as defined for Formula (I*).

In some embodiments, the compound of Formula (I*) is of Formula (IAC):

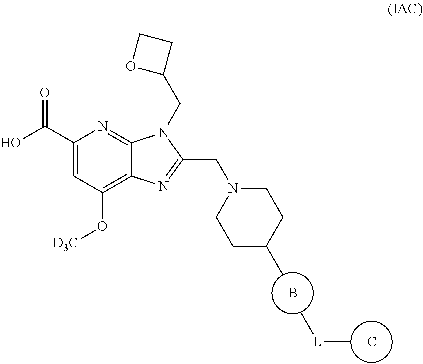

or a pharmaceutically acceptable salt thereof;
wherein Ring B, L, and Ring C are as defined for Formula (I*).

In some embodiments, the compound of Formula (I*) is of Formula (LAD):

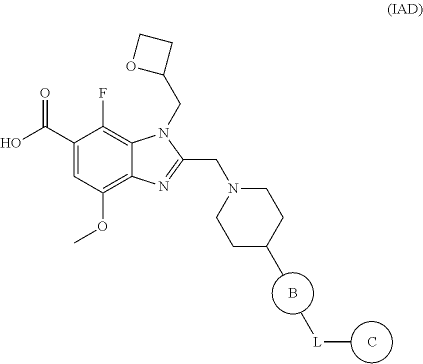

or a pharmaceutically acceptable salt thereof;
wherein Ring B, L, and Ring C are as defined for Formula (I*).

In some embodiments, the compound of Formula (I*) is of Formula (IAE):

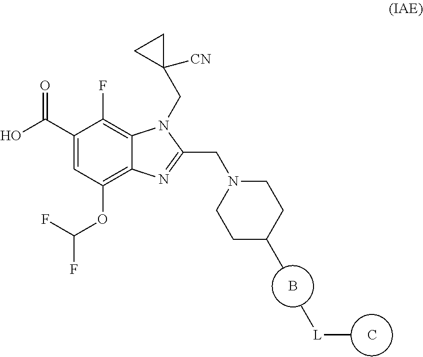

or a pharmaceutically acceptable salt thereof;
wherein Ring B, L, and Ring C are as defined for Formula (I*).

In some embodiments, X³ is N. In some embodiments, X³ is CH.

In some embodiments, X⁶ is N. In some embodiments, X⁶ is CR⁴.

In some embodiments, R¹ is R¹ is —O—C₁₋₆ alkyl. In some embodiments, the alkyl group is linear. In some embodiments, the alkyl group is branched. In some embodiments, the alkyl group is linear and is optionally substituted with one or more deuterium, —CN or —O—C₁₋₆ alkyl. In some embodiments, the alkyl group is unsubstituted. In some embodiments, the alkyl group is substituted with one or more —CN or —O—C₁₋₆ alkyl. In some embodiments, R¹ is —O—CH₂—CH₃, —O—CH₃, —O—CH₂—CN, —O—CH₂—CH₂—O—CH₃, or —O—CH₂CH(—O—CH₃)—CH₃. In some embodiments, R¹ is branched and optionally substituted with one or more —CN. In some embodiments, R¹ is

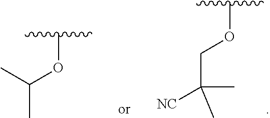

In some embodiments, R¹ is —O—C₁₋₆ haloalkyl, wherein the haloalkyl group is linear. In some embodiments, the haloalkyl group is substituted with one or more fluorine. In some embodiments, R¹ is —O—CHF₂, —O—CF₃, —O—CH₂—CH₂F, —O—CH₂—CF₃, —O—CH₂—CHF—CH₃, —O—CHF—CH₃, —O—CHF—CH₂F, or —O—CH₂—CH₂F.

In some embodiments, R¹ is —O—C₃₋₁₀ cycloalkyl optionally substituted with C₁₋₆ alkoxy or halogen. In some embodiments, R¹ is —O-cyclopropyl or —O-cyclobutyl, wherein the cyclopropyl or cyclobutyl is optionally substituted with fluorine or methoxy. In some embodiments, R¹ is —O—C₃₋₁₀ cycloalkyl optionally substituted with one or more halogen, e.g., fluorine, —OCH₃, or —CN.

In some embodiments, R¹ is —O-(3- to 8-membered heterocyclyl). In some embodiments, the 3- to 8-membered heterocyclyl comprises one oxygen atom. In some embodiments, the 3- to 8-membered heterocyclyl comprises two oxygen atoms. In some embodiments, the R¹ is —O—CH₂-(1,4-dioxan-2-yl) or —O—CH₂-tetrahydrofuran-2-yl.

In some embodiments, R¹ is —O—(C₁₋₆ alkyl)-(C₃₋₁₀ cycloalkyl). In some embodiments, R¹ is —O—(C₁₋₆ alkyl)-(C₃₋₁₀ cycloalkyl) optionally substituted with one or more halogen, cyano, or —OCH₃. In some embodiments, R¹ is

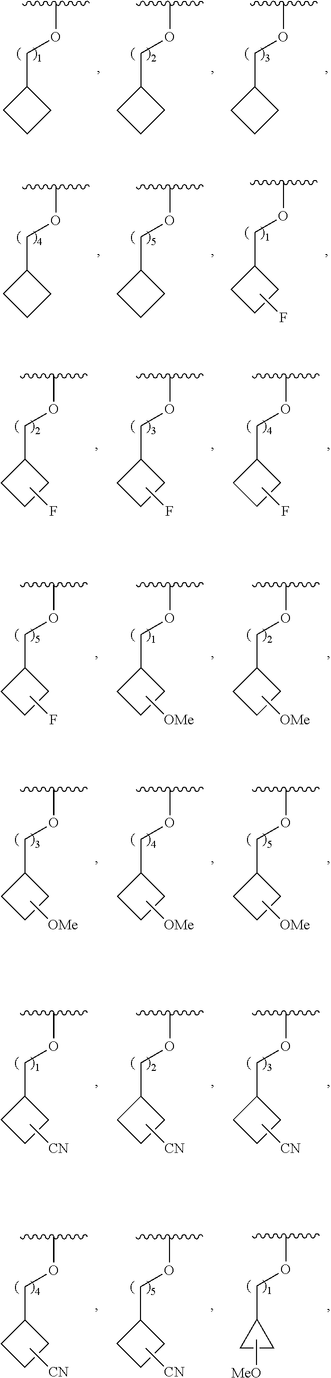

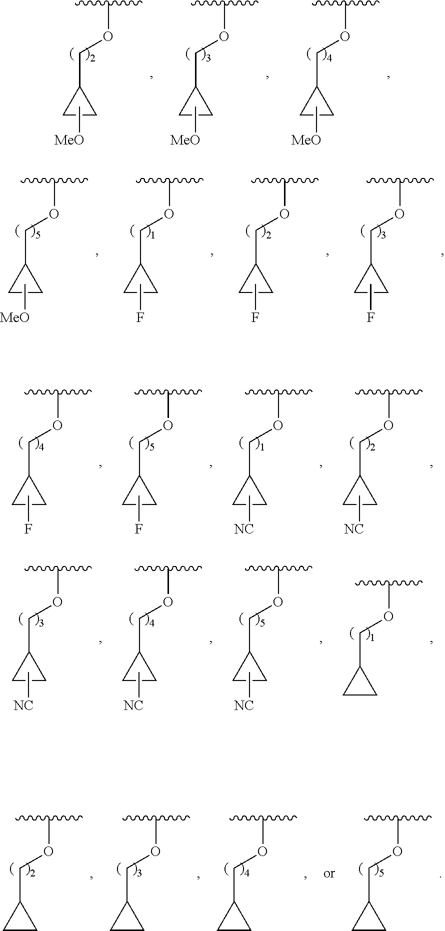

In some embodiments, R¹ is

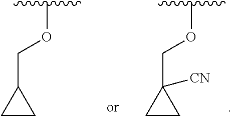 [SMILES: *OCC1CC1 ; *OCC1(C#N)CC1]

In some embodiments, R¹ is —O—(C₁₋₆ alkyl)-(3- to 8-membered heterocyclyl). In some embodiments, —O—(C₁₋₆ alkyl)-(3- to 8-membered heterocyclyl) optionally substituted with one or more halogen, cyano, or —OCH₃. In some embodiments, R¹ is

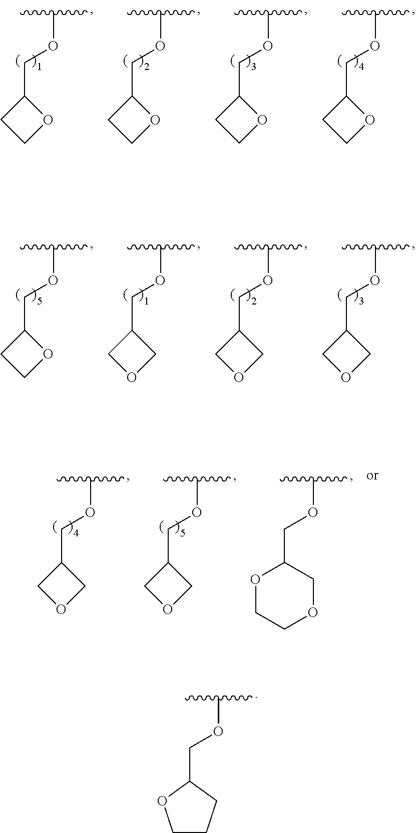

In some embodiments, R¹

is

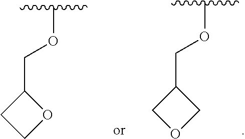 [SMILES: *OCC1CCO1 ; *OCC1COC1]

In some embodiments, R¹ is

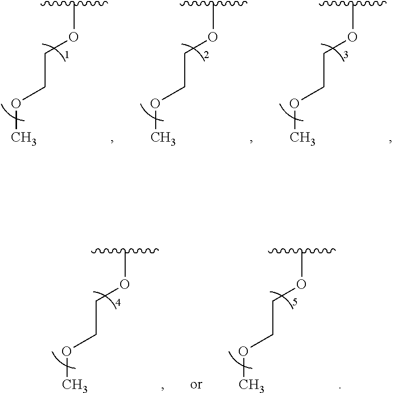

In some embodiments, R¹ is C₁₋₆ haloalkyl. In some embodiments, R¹ is —CF₂—CH₃.

In some embodiments, R¹ is —O-(3- to 8-membered heterocyclyl). In some embodiments, R¹ is oxetanyl. In some embodiments, R¹ is oxetan-3-yl.

In some embodiments, R¹ is —O—(CH₂—CH(—OCH₃)—CH₂—O)₁₋₅—CH₃. In some embodiments, R¹ is

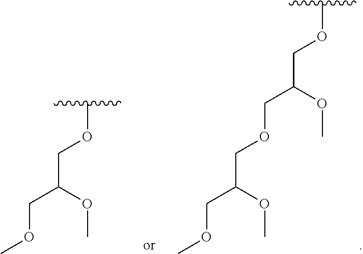

In some embodiments, R¹ is —NR⁸R⁹. In some embodiments, R⁸ and R⁹ combine with the atom to which they are attached to form a 6-membered heterocyclyl. In some embodiments, R⁸ and R⁹ combine with the atom to which they are attached to form morpholine. In some embodiments, R⁸ and R⁹ are hydrogen. In some embodiments, R¹ is NH₂. In some embodiments, R¹ is NR⁸R⁹, wherein R⁸ is H and R⁹ is C₁₋₆ alkyl optionally substituted with oxo.

In some embodiments, R² is C₃₋₁₀ cycloalkyl optionally substituted with one or more —CN, C₁₋₆ haloalkyl, or C₁₋₆ alkyl optionally substituted with one or more —CN. In some embodiments, R² is cyclopropyl optionally substituted with one or more —CN, C₁₋₆ haloalkyl, or C₁₋₆ alkyl optionally substituted with one or more —CN. In some embodiments, R² is

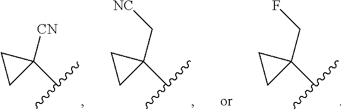 [SMILES: *C1(C#N)CC1 ; *C1(CC#N)CC1 ; *C1(CF)CC1]

In some embodiments, R² is 4 or 5-membered heterocyclyl comprising at least one oxygen or at least one sulfur atom, wherein the 4- or 5-membered heterocyclyl is optionally substituted with one or more oxo,

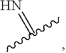 [SMILES: *=N]

or C₁₋₆ alkyl. In some embodiments, R² is (i) thietane optionally substituted with one or more oxo or

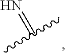 [SMILES: *=N]

(ii) oxetane, or (iii) tetrahydrofuran optionally substituted with one or more C16 alkyl. In some embodiments, R² is

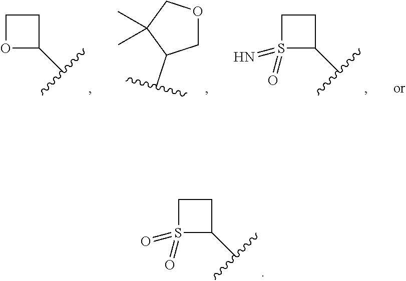

In some embodiments, R² is 5-membered heteroaryl, comprising 1 or 2 heteroatoms independently selected from N, and S, wherein at least one heteroatom of R⁵ is S. In some embodiments, R² is thiazole. In some embodiments, R² is

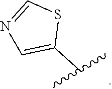 [SMILES: *c1cncs1]

In some embodiments, R² is H. In some embodiments, R² is —OCH₃.

In some embodiments, n is 1.

In some embodiments, X¹ is

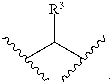

In some embodiments, X¹ is

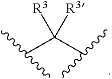

In some embodiments, X¹ is

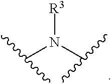

In some embodiments, R³ is methyl. In some embodiments, R³ is hydrogen. In some embodiments, R³ and R^{3′} independently are CH₃, CD₃, deuterium, or hydrogen. In some embodiments, X¹ is

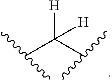 [SMILES: *C*]

In some embodiments, X¹ is

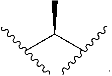

In some embodiments, X¹ is

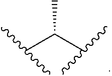

In some embodiments, X¹ is

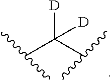 [SMILES: *C(*)([2H])[2H]]

In some embodiments, Ring A is

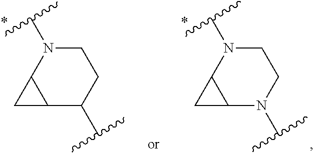 [SMILES: *C1CCN(*)C2CC12 ; *N1CCN(*)C2CC21]

wherein * indicates attachment to X¹. In some embodiments, Ring A is

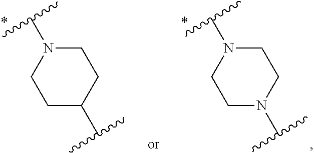 [SMILES: *C1CCN(*)CC1 ; *N1CCN(*)CC1]

wherein * indicates attachment to X¹. In some embodiments, Ring A is a phenyl ring optionally substituted with one or more halogen or C₁₋₆ alkyl. In some embodiments, Ring A is

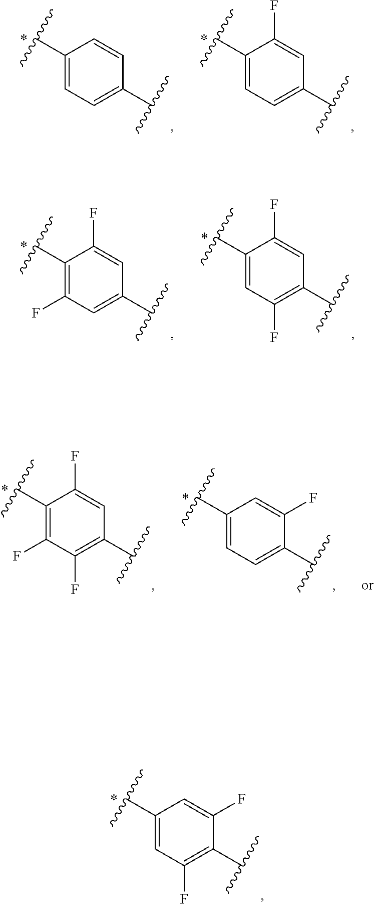

wherein [symbol] indicates attachment to X¹. In some embodiments, Ring A is a 6-membered heteroaryl ring optionally substituted with one or more halogen. In some embodiments, Ring A is

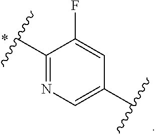 [SMILES: *c1cnc(*)c(F)c1]

In some embodiments, Ring A is

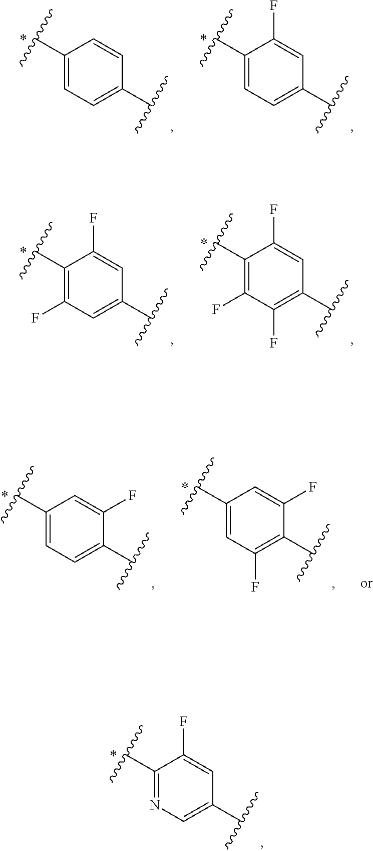

wherein [symbol] indicates attachment to X¹.

In some embodiments, Ring B is a 6-membered heteroarylene comprising nitrogen. In some embodiments, Ring B is pyridinylene or pyrimidinylene optionally substituted with one or more halogen. In some embodiments, Ring B is pyridinylene. In some embodiments, Ring B is

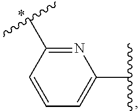 [SMILES: *c1cccc(*)n1]

wherein * indicates attachment to Ring A or L′. In some embodiments, Ring B is

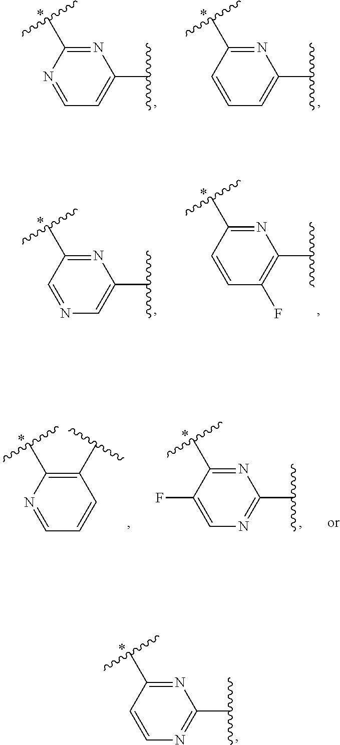

wherein * indicates attachment to Ring A or L′.

In some embodiments, Ring B is a 9-membered heterocycylene comprising two oxygen atoms optionally substituted with one or more C₁₋₆ alkyl. In some embodiments, Ring B is benzoidoxolylene optionally substituted with one or more C₁₋₆ alkyl. In some embodiments, Ring B is

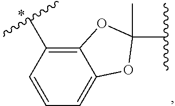 [SMILES: *c1cccc2c1OC(*)(C)O2]

wherein * indicates attachment to Ring A or L′. In some embodiments, Ring B is B is a 10-membered heterocycylene comprising two oxygen atoms. In some embodiments, Ring B is benzodioxanylene. In some embodiments, Ring B is

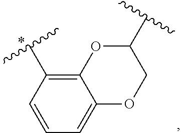 [SMILES: *c1cccc2c1OC(*)CO2]

wherein * indicates attachment to Ring A or L′. In some embodiments, Ring B is

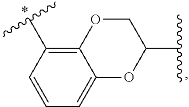 [SMILES: *c1cccc2c1OCC(*)O2]

wherein * indicates attachment to Ring A or L′.

In some embodiments, Ring B is a 10-membered heterocyclene comprising one oxygen atom and one nitrogen atom. In some embodiments, Ring B is

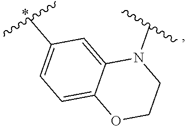 [SMILES: *c1ccc2c(c1)N(*)CCO2]

wherein * indicates attachment to Ring A or L′.

In some embodiments, L is a bond. In some embodiments, L is *—O—CH₂—, wherein * indicates attachment to Ring B.

In some embodiments, Ring C is phenyl optionally substituted with one or more halogen, —CN, —OCH₃,

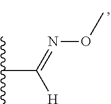 [SMILES: */C=N/OC]

or cyclopropyl. In some embodiments, Ring C is

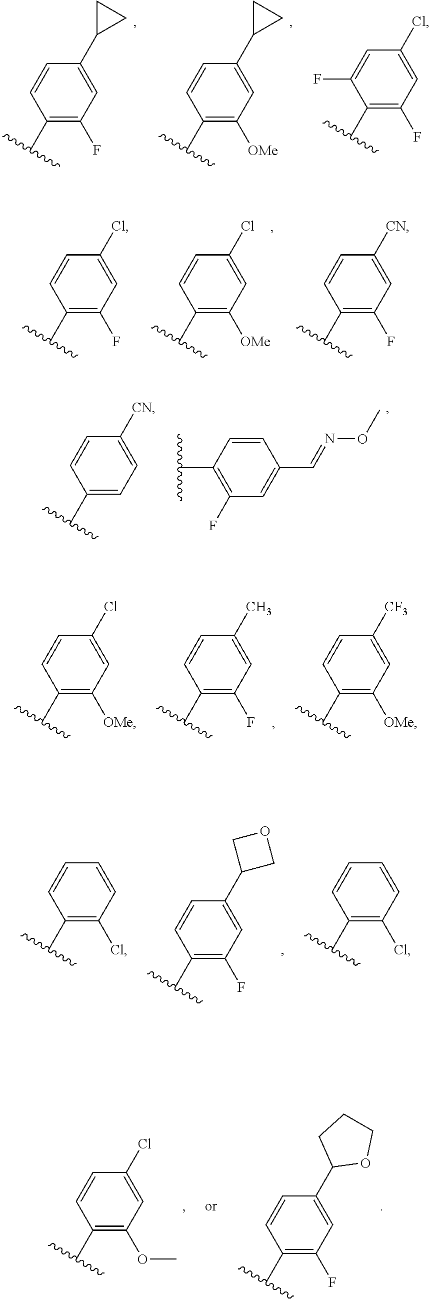

In some embodiments, Ring C is a 6-membered heteroaryl comprising a nitrogen atom optionally substituted with one or more halogen, —CN, —O—C₁₋₆ alkyl, —C(═O)—(C₃₋₁₀ cycloalkyl), or C₃₋₁₀ cycloalkyl. In some embodiments, Ring C is pyridinyl optionally substituted with one or more —Cl, —F, —CN, —OCH₃, cyclopropyl,

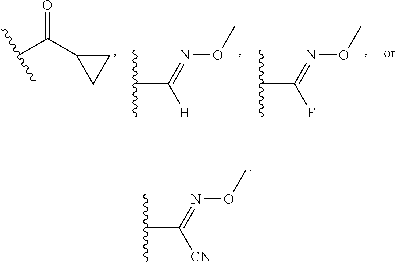

In some embodiments, Ring C is

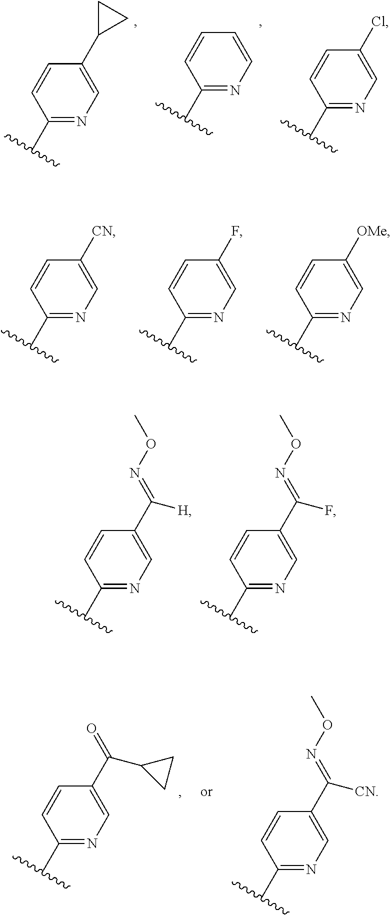

In some embodiments, Ring C is a bicylic 9- or 10-membered heteroaryl comprising two nitrogen atoms optionally substituted with one or more C₁₋₆ alkyl, or oxo. In some embodiments, Ring C is pyrazolopyridine, triazolopyridine, 2,3-dihydro-1H-pyrrolopyridine, 1,2-dihydrooxazolopyridine, 1,2,3,4-tetrahydronaphthyridine, 2,3-dihydro-pyridooxazine, 2,3-dihydro-1H-pyrrolopyridine, or 2,3-dihydrooxazolopyridine, wherein the pyrazolopyridine, triazolopyridine, 2,3-dihydro-1H-pyrrolopyridine, 1,2-dihydrooxazolopyridine, 1,2,3,4-tetrahydronaphthyridine, 2,3-dihydro-pyridooxazine, 2,3-dihydro-1H-pyrrolopyridine, or 2,3-dihydrooxazolopyridine is optionally substituted with one or more C₁₋₆ alkyl or oxo. In some embodiments, Ring C is

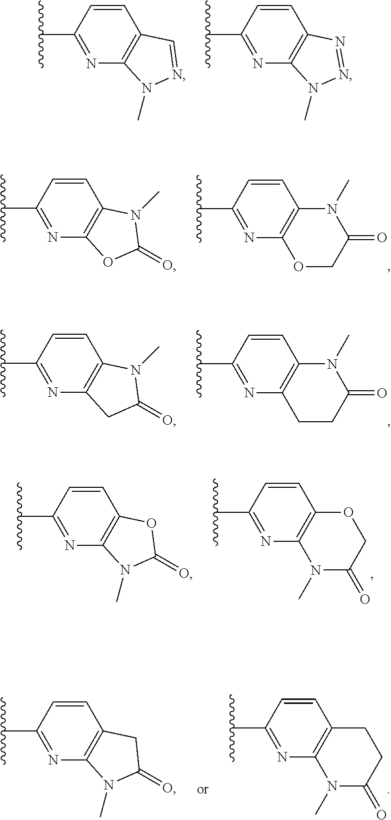

In some embodiments,

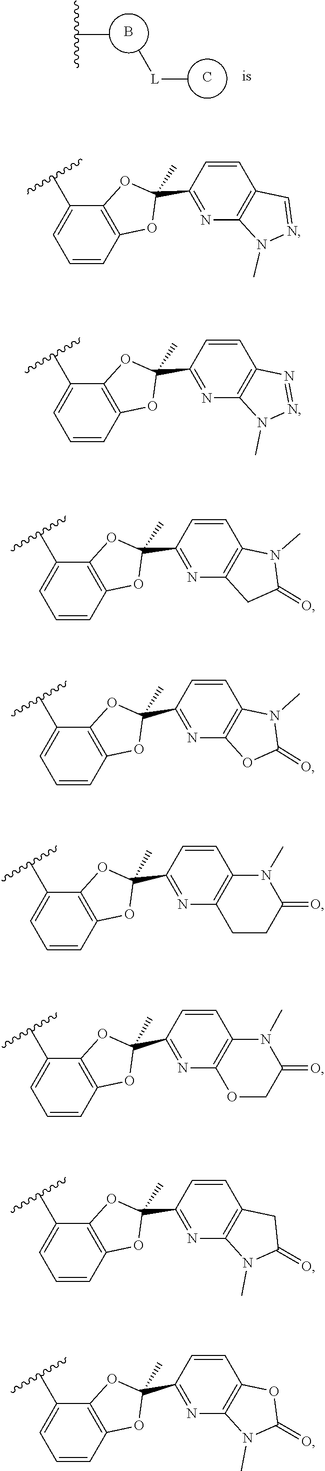

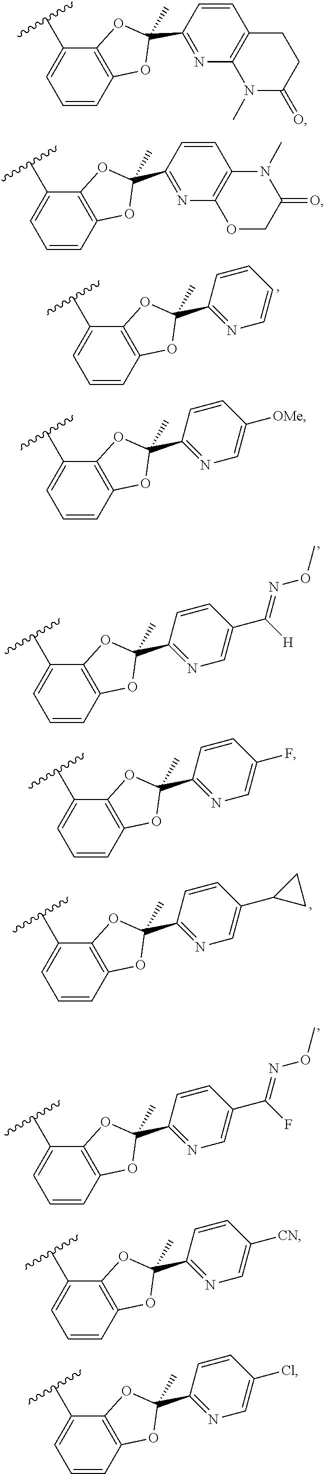

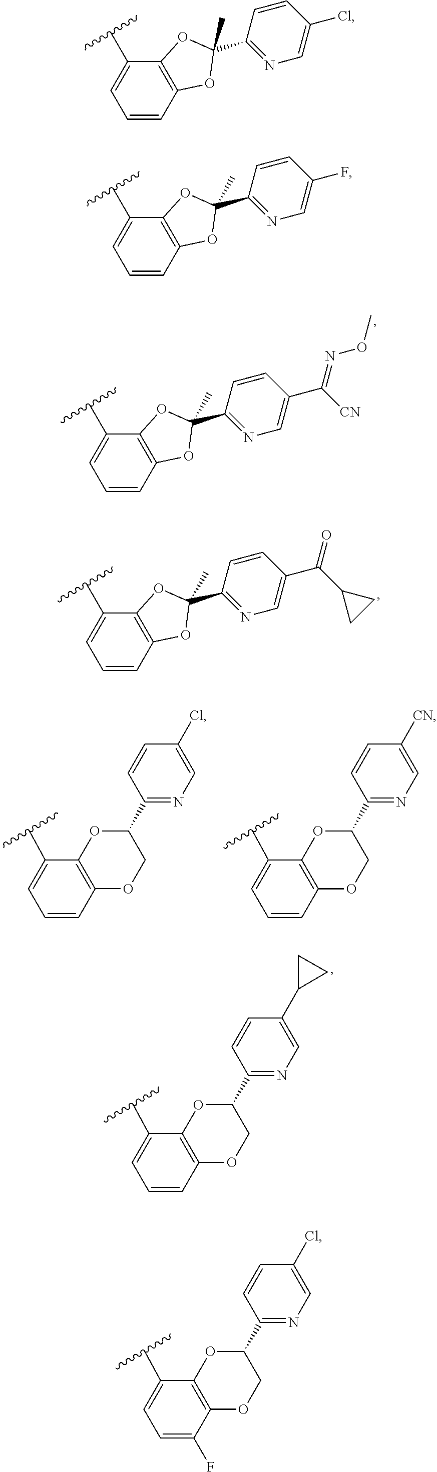

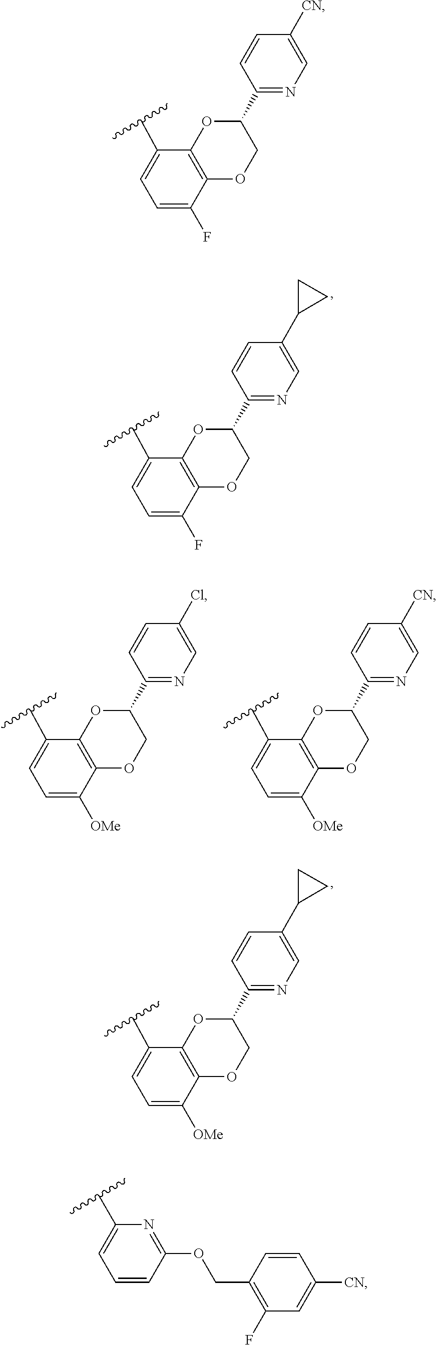 [SMILES: *c1ccc(F)c2c1O[C@H](c1ccc(C#N)cn1)CO2 ; *c1ccc(F)c2c1O[C@H](c1ccc(C3CC3)cn1)CO2 ; *c1ccc(OC)c2c1O[C@H](c1ccc(Cl)cn1)CO2 ; *c1ccc(OC)c2c1O[C@H](c1ccc(C#N)cn1)CO2 ; *c1ccc(OC)c2c1O[C@H](c1ccc(C3CC3)cn1)CO2 ; *c1cccc(OCc2ccc(C#N)cc2F)n1]

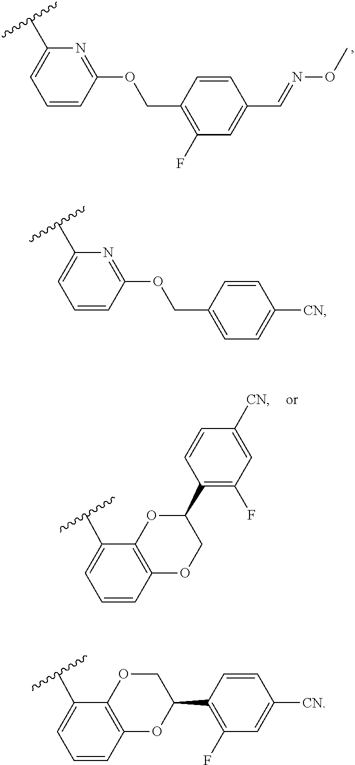 [SMILES: *c1cccc(OCc2ccc(/C=N/OC)cc2F)n1 ; *c1cccc(OCc2ccc(C#N)cc2)n1 ; *c1cccc2c1O[C@@H](c1ccc(C#N)cc1F)CO2 ; *c1cccc2c1OC[C@@H](c1ccc(C#N)cc1F)O2]

In some embodiments, R¹² is —COOH. In some embodiments, R¹² is

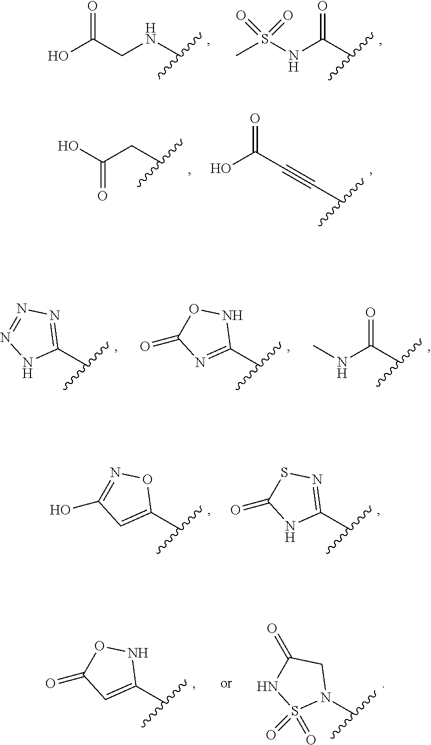

In some embodiments, the compound of Formula (I**) is of Formula (II**):

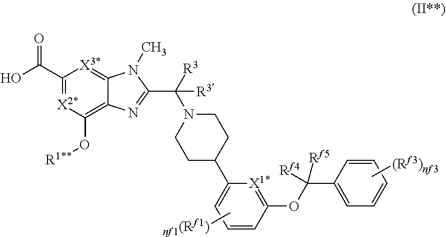

or a pharmaceutically acceptable salt thereof; wherein
R^{f4} and R^{f5} are each independently selected from C₁₋₆ alkyl, H and D
nf1 is 0, 1, 2, 3, or 4;
nf3 is 0, 1, 2, 3, 4, or 5;
each R^{f1} is halogen;
R³ and R^{3′} independently are H or D;
X¹* is N or CR^{f1};
X^{2*} and X^{3*} independently are CH or CF;
each R^{f3} is independently selected from halogen, —CN, C₁₋₆ alkyl, C₁₋₆ haloalkyl, C₁₋₆ alkoxy, or —C(O)N(R^{f3′})₂,
each R^{f3′} is independently selected from H or C₁₋₆ alkyl; and
R^{1**} is H or C₁₋₂ alkyl optionally substituted with one or more deuterium or halogen.

In some embodiments, the compound of Formula (I**) is of Formula (II*)

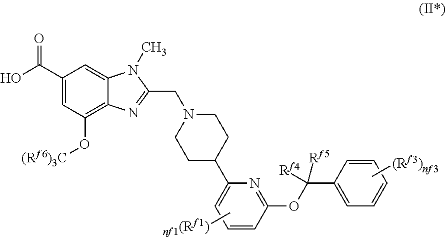

or a pharmaceutically acceptable salt thereof;
each R^{f1} is independently selected from halogen
R^{f4} and R^{f5} are each independently selected from C₁₋₆ alkyl, H and D
nf1 is 0, 1, 2, or 3;
nf3 is 0, 1, 2, 3, 4, or 5;
each R^{f1} is halogen;
each R^{f3} is independently selected from halogen, —CN, C₁₋₆ alkyl, C₁₋₆ haloalkyl, C₁₋₆ alkoxy, or —C(O)N(R^{f3′})₂,
   each R^{f3′} is independently selected from H or C₁₋₆ alkyl;
each R^{f6} is independently selected from H, D or F.

In some embodiments, R^{f4} and R^{f5} are each independently selected from CH₃, H, and D.

In some embodiments, each R^{f1} is fluorine.

In some embodiments, the present disclosure provides a pharmaceutical composition comprising the compound of any one of the compounds disclosed herein, or a pharmaceutically acceptable salt thereof, and a pharmaceutically acceptable excipient.

In some embodiments, the present disclosure provides a method of treating a disease mediated by glucagon-like peptide-1 receptor (GLP-1R) in an individual in need thereof, comprising administering to the individual a therapeutically effective amount of any one of the compounds disclosed herein, or a pharmaceutically acceptable salt thereof, or any pharmaceutical composition disclosed herein.

In some embodiments, the disease is a liver disease. In some embodiments, the liver disease is primary biliary cirrhosis (PBC), primary sclerosing cholangitis (PSC), drug induced cholestasis, intrahepatic cholestasis of pregnancy, parenteral nutrition associated cholestasis (PNAC), bacterial overgrowth or sepsis associated cholestasis, autoimmune hepatitis, viral hepatitis, alcoholic liver disease, nonalcoholic fatty liver disease (NAFLD), nonalcoholic steatohepatitis (NASH), graft versus host disease, transplant liver regeneration, congenital hepatic fibrosis, choledocholithiasis, granulomatous liver disease, intra- or extrahepatic malignancy, Sjogren's syndrome, sarcoidosis, Wilson's disease, Gaucher's disease, hemochromatosis, or oti-antitrypsin deficiency.

In some embodiments, the disease is diabetes.

In some embodiments, the disease is cardiometabolic disease.

In some embodiments, the disease is obesity.

In some embodiments, the present disclosure provides the use any one of the compounds disclosed herein, or a pharmaceutically acceptable salt thereof, in the manufacture of a medicament for treating a disease mediated by GLP-1R.

In some embodiments, the present disclosure provides a method of treating obesity in an individual in need thereof, comprising administering to the individual a compound disclosed herein, or pharmaceutically acceptable salt thereof, or a pharmaceutical composition disclosed herein.

In some embodiments, the present disclosure provides the use of a compound disclosed herein, or a pharmaceutically acceptable salt thereof, or a pharmaceutical composition disclosed herein, in the manufacture of a medicament for treating obesity.

In some embodiments, the present disclosure provides the use of a compound disclosed herein, or a pharmaceutically acceptable salt thereof, or a pharmaceutical composition disclosed herein, for treating obesity in an individual in need thereof.

In some embodiments, the present disclosure provides a method of decreasing food intake in an individual in need thereof, comprising administering to the individual any one of the compounds disclosed herein, or pharmaceutically acceptable salt thereof, or any pharmaceutical composition herein.

In some embodiments, the present disclosure provides a method of increasing glucose tolerance in an individual in need thereof, comprising administering to the individual any one of the compounds disclosed herein, or pharmaceutically acceptable salt thereof, or any pharmaceutical composition herein.

In some aspects, the present disclosure provides a compound obtainable by, or obtained by, a method for preparing a compound as described herein (e.g., a method comprising one or more steps described in Schemes A to W).

In some aspects, the present disclosure provides an intermediate as described herein, being suitable for use in a method for preparing a compound as described herein (e.g., the intermediate is selected from the intermediates described in Examples A1 to A9.

In some aspects, the present disclosure provides a method of modulating GLP-1R activity (e.g., in vitro or in vivo), comprising contacting a cell with an effective amount of a compound of the present disclosure or a pharmaceutically acceptable salt thereof.

In some aspects, the present disclosure provides a method of treating or preventing a disease or disorder disclosed herein in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a disease or disorder disclosed herein in a subject in need thereof, comprising administering to the subject a therapeutically effective amount of a compound of the present disclosure, or a pharmaceutically acceptable salt thereof, or a pharmaceutical composition of the present disclosure.

In some aspects, the present disclosure provides a compound of the present disclosure or a pharmaceutically acceptable salt thereof for use in modulating GLP-1R activity (e.g., in vitro or in vivo).

In some aspects, the present disclosure provides a compound of the present disclosure or a pharmaceutically acceptable salt thereof for use in treating or preventing a disease or disorder disclosed herein.

In some aspects, the present disclosure provides a compound of the present disclosure or a pharmaceutically acceptable salt thereof for use in treating a disease or disorder disclosed herein.

In some aspects, the present disclosure provides use of a compound of the present disclosure or a pharmaceutically acceptable salt thereof in the manufacture of a medicament for modulating GLP-1R activity (e.g., in vitro or in vivo).

In some aspects, the present disclosure provides use of a compound of the present disclosure or a pharmaceutically acceptable salt thereof in the manufacture of a medicament for treating or preventing a disease or disorder disclosed herein.

In some aspects, the present disclosure provides use of a compound of the present disclosure or a pharmaceutically acceptable salt thereof in the manufacture of a medicament for treating a disease or disorder disclosed herein.

In some aspects, the present disclosure provides a method of preparing a compound of the present disclosure.

In some aspects, the present disclosure provides a method of preparing a compound, comprising one or more steps described herein.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

Other features and advantages of the disclosure will be apparent from the following detailed description and claims.

### BRIEF DESCRIPTION OF THE DRAWINGS

FIG. 1 provides plasma concentrations of Compound 1 in rats after IV bolus dosing with 3 mg/kg.

FIG. 2 provides plasma concentrations of Compound 1 in rats after oral dosing with 10 mg/kg.

FIG. 3 provides mean plasma concentrations for compound 1 after IV bolus dosing at 3.0 mg/kg and IV bolus dosing at 10 mg/kg.

### DETAILED DESCRIPTION

The present disclosure relates to compounds, prodrugs, and pharmaceutically acceptable salts thereof, which may modulate GLP-1R activity and are accordingly useful in methods of treatment of the human or animal body, e.g., in the treatment of a disease mediated by GLP-1R. The present disclosure also relates to processes for the preparation of these compounds, to pharmaceutical compositions comprising them and to their use in the treatment of disorders in which GLP-1R is implicated, such as diabetes, non-alcoholic fatty liver disease (NASH), obesity, hyperglycemia, and/or insulinoma.

### Definitions

Unless otherwise stated, the following terms used in the specification and claims have the following meanings set out below.

Without wishing to be limited by this statement, it is understood that, while various options for variables are described herein, the disclosure intends to encompass operable embodiments having combinations of the options. The disclosure may be interpreted as excluding the non-operable embodiments caused by certain combinations of the options.

As used herein, “alkyl”, “C₁, C₂, C₃, C₄, C₅ or C₆ alkyl” or “C₁-C₆ alkyl” is intended to include C₁, C₂, C₃, C₄, C₅ or C₆ straight chain (linear) saturated aliphatic hydrocarbon groups and C₃, C₄, C₅ or C₆ branched saturated aliphatic hydrocarbon groups. For example, C₁-C₆ alkyl is intends to include C₁, C₂, C₃, C₄, C₅ and C₆ alkyl groups. Examples of alkyl include, moieties having from one to six carbon atoms, such as, but not limited to, methyl, ethyl, n-propyl, i-propyl, n-butyl, s-butyl, t-butyl, n-pentyl, i-pentyl, or n-hexyl. In some embodiments, a straight chain or branched alkyl has six or fewer carbon atoms (e.g., C₁-C₆ for straight chain, C₃-C₆ for branched chain), and in another embodiment, a straight chain or branched alkyl has four or fewer carbon atoms. A divalent alkyl group is referred to herein as “alkylene.”

As used herein, the term “optionally substituted alkyl” refers to unsubstituted alkyl or alkyl having designated substituents replacing one or more hydrogen atoms on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

As used herein, the term “alkenyl” includes unsaturated aliphatic groups analogous in length and possible substitution to the alkyls described above, but that contain at least one double bond. For example, the term “alkenyl” includes straight chain alkenyl groups (e.g., ethenyl, propenyl, butenyl, pentenyl, hexenyl, heptenyl, octenyl, nonenyl, decenyl), and branched alkenyl groups. In certain embodiments, a straight chain or branched alkenyl group has six or fewer carbon atoms in its backbone (e.g., C₂-C₆ for straight chain, C₃-C₆ for branched chain). The term “C₂-C₆” includes alkenyl groups containing two to six carbon atoms. The term “C₃-C₆” includes alkenyl groups containing three to six carbon atoms. A divalent alkenyl group is referred to herein as “alkenylene.”

As used herein, the term “optionally substituted alkenyl” refers to unsubstituted alkenyl or alkenyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, heterocyclyl, alkylaryl, or an aromatic or heteroaromatic moiety.

As used herein, the term “alkynyl” includes unsaturated aliphatic groups analogous in length and possible substitution to the alkyls described above, but which contain at least one triple bond. For example, “alkynyl” includes straight chain alkynyl groups (e.g., ethynyl, propynyl, butynyl, pentynyl, hexynyl, heptynyl, octynyl, nonynyl, decynyl), and branched alkynyl groups. In certain embodiments, a straight chain or branched alkynyl group has six or fewer carbon atoms in its backbone (e.g., C₂-C₆ for straight chain, C₃-C₆ for branched chain). The term “C₂-C₆” includes alkynyl groups containing two to six carbon atoms. The term “C₃-C₆” includes alkynyl groups containing three to six carbon atoms. As used herein, “C₂-C₆ alkenylene linker” or “C₂-C₆ alkynylene linker” is intended to include C₂, C₃, C₄, C₅ or C₆ chain (linear or branched) divalent unsaturated aliphatic hydrocarbon groups. For example, C₂-C₆ alkenylene linker is intended to include C₂, C₃, C₄, C₅ and C₆ alkenylene linker groups. A divalent alkynyl group is referred to herein as “alkynylene.”

As used herein, the term “optionally substituted alkynyl” refers to unsubstituted alkynyl or alkynyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or Heteroaromatic moiety.

Other optionally substituted moieties (such as optionally substituted cycloalkyl, heterocyclyl, aryl, or heteroaryl) include both the unsubstituted moieties and the moieties having one or more of the designated substituents. For example, substituted heterocyclyl includes those substituted with one or more alkyl groups, such as 2,2,6,6-tetramethyl-piperidinyl and 2,2,6,6-tetramethyl-1,2,3,6-tetrahydropyridinyl.

As used herein, the term “cycloalkyl” refers to a saturated or partially unsaturated hydrocarbon monocyclic or polycyclic (e.g., fused, bridged, or spiro rings) system having 3 to 30 carbon atoms (e.g., C₃-C₁₂, C₃-C₁₀, or C₃-C₈). Examples of cycloalkyl include, but are not limited to, cyclopropyl, cyclobutyl, cyclopentyl, cyclohexyl, cycloheptyl, cyclooctyl, cyclopentenyl, cyclohexenyl, cycloheptenyl, 1,2,3,4-tetrahydronaphthalenyl, and adamantyl. In the case of polycyclic cycloalkyl, only one of the rings in the cycloalkyl needs to be non-aromatic. A divalent cycloalkyl group is referred to herein as “cycloalkylene.”

As used herein, the term “heterocyclyl” refers to a saturated or partially unsaturated 3-8 membered monocyclic, 7-12 membered bicyclic (fused, bridged, or spiro rings), or 11-14 membered tricyclic ring system (fused, bridged, or spiro rings) having one or more heteroatoms (such as O, N, S, P, or Se), e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur, unless specified otherwise. Examples of heterocyclyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, triazolidinyl, oxiranyl, azetidinyl, oxetanyl, thietanyl, 1,2,3,6-tetrahydropyridinyl, tetrahydropyranyl, dihydropyranyl, pyranyl, morpholinyl, tetrahydrothiopyranyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl, 1,4-dioxaspiro[4.5]decanyl, 1-oxaspiro[4.5]decanyl, 1-azaspiro[4.5]decanyl, 3′H-spiro[cyclohexane-1,1′-isobenzofuran]-yl, 7′H-spiro[cyclohexane-1,5′-furo[3,4-b]pyridin]-yl, 3′H-spiro[cyclohexane-1,1′-furo[3,4-c]pyridin]-yl, 3-azabicyclo[3.1.0] hexanyl, 3-azabicyclo[3.1.0] hexan-3-yl, 1,4,5,6-tetrahydropyrrolo[3,4-c]pyrazolyl, 3,4,5,6,7,8-hexahydropyrido[4,3-d]pyrimidinyl, 4,5,6,7-tetrahydro-1H-pyrazolo[3,4-c]pyridinyl, 5,6,7,8-tetrahydropyrido[4,3-d]pyrimidinyl, 2-azaspiro[3.3]heptanyl, 2-methyl-2-azaspiro[3.3]heptanyl, 2-azaspiro[3.5]nonanyl, 2-methyl-2-azaspiro[3.5]nonanyl, 2-azaspiro[4.5]decanyl, 2-methyl-2-azaspiro[4.5]decanyl, 2-oxa-azaspiro[3.4]octanyl, 2-oxa-azaspiro[3.4]octan-6-yl, 5,6-dihydro-4H-cyclopenta[b]thiophenyl, and the like. In the case of multicyclic heterocyclyl, only one of the rings in the heterocyclyl needs to be non-aromatic (e.g., 4,5,6,7-tetrahydrobenzo[c] isoxazolyl, benzo[d][1,3]dioxole, 2,3-dihydrobenzo[b][1,4]dioxine, 2,3-dihydrobenzofuran, and the like). A divalent heterocyclyl group is referred to herein as “heterocyclylene.”

It is understood that when a variable has two attachments to the rest of the formula of the compound, the two attachments could be at the same atom or different atoms of the variable, as allowed by valency. For example, when a variable (e.g., variable X) is cycloalkyl or heterocyclyl, and has two attachments to the rest of the formula of the compound, the two attachments could be at the same atom or different atoms of the cycloalkyl or heterocyclyl.

As used herein, the term “aryl” refers to groups monocyclic or multicyclic systems with one or more aromatic rings that do not contain any heteroatom in the ring structure(s). The term aryl includes both monovalent species and divalent species. Examples of aryl groups include, but are not limited to, phenyl, biphenyl, naphthyl and the like. For example, an aryl is phenyl. A divalent aryl group is referred to herein as “arylene.”

As used herein, the term “heteroaryl” refers to a stable 5-, 6-, or 7-membered monocyclic or 7-, 8-, 9-, 10-, 11- or 12-membered bicyclic aromatic heterocyclic ring which consists of carbon atoms and one or more heteroatoms, e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur. The nitrogen atom may be substituted or unsubstituted (i.e., N or NR wherein R is H or other substituents, as defined). The nitrogen and sulfur heteroatoms may optionally be oxidised (i.e., N→O and S(O)ₚ, where p=1 or 2). It is to be noted that total number of S and O atoms in the aromatic heterocycle is not more than 1. Examples of heteroaryl groups include pyrrole, furan, thiophene, thiazole, isothiazole, imidazole, triazole, tetrazole, pyrazole, oxazole, isoxazole, isothiazole, pyridine, pyrazine, purine, pyridazine, pyrimidine, and the like. Heteroaryl groups can also be fused or bridged with alicyclic or heterocyclic rings, which are not aromatic so as to form a multicyclic system (e.g., 4,5,6,7-tetrahydrobenzo[c] isoxazolyl, 2,3-dihydro-1H-pyrrolopyridine, 1,2,3,4-tetrahydronaphthyridine, 2,3-dihydro-pyridooxazine, 2,3-dihydro-1H-pyrrolopyridine, 2,3-dihydrooxazolopyridine, and the like). A divalent heteroaryl group is referred to herein as “heteroarylene.”

Furthermore, the terms “aryl” and “heteroaryl” include multicyclic aryl and heteroaryl groups, respectively, e.g., tricyclic, bicyclic, e.g., naphthalene, benzoxazole, benzodioxazole, benzothiazole, benzoimidazole, benzothiophene, quinoline, isoquinoline, naphthrydine, indole, benzofuran, purine, benzofuran, deazapurine, indolizine.

The cycloalkyl, heterocyclyl, aryl, or heteroaryl ring can be substituted at one or more ring positions (e.g., the ring-forming carbon or heteroatom such as N) with such substituents as described above, for example, alkyl, alkenyl, alkynyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl and heteroaryl groups can also be fused or bridged with alicyclic or heterocyclic rings, which are not aromatic so as to form a multicyclic system (e.g., tetralin, methylenedioxyphenyl such as benzo[d][1,3]dioxole-5-yl).

As used herein, the term “substituted,” means that any one or more hydrogen atoms on the designated atom is replaced with a selection from the indicated groups, provided that the designated atom's normal valency is not exceeded, and that the substitution results in a stable compound. When a substituent is oxo or keto (i.e., ═O), then 2 hydrogen atoms on the atom are replaced. Keto substituents are not present on aromatic moieties. Ring double bonds, as used herein, are double bonds that are formed between two adjacent ring atoms (e.g., C═C, C═N or N═N).

“Stable compound” and “stable structure” are meant to indicate a compound that is sufficiently robust to survive isolation to a useful degree of purity from a reaction mixture, and formulation into an efficacious therapeutic agent.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., R) occurs more than one time in any constituent or formula for a compound, its definition at each occurrence is independent of its definition at every other occurrence. Thus, for example, if a group is shown to be substituted with 0-2 R moieties, then the group may optionally be substituted with up to two R moieties and R at each occurrence is selected independently from the definition of R. Also, combinations of substituents and/or variables are permissible, but only if such combinations result in stable compounds.

As used herein, the term “hydroxy” or “hydroxyl” includes groups with an —OH or —O⁻.

As used herein, the term “halo” or “halogen” refers to fluoro, chloro, bromo and iodo.

The term “haloalkyl” or “haloalkoxyl” refers to an alkyl or alkoxyl substituted with one or more halogen atoms.

The term “heteroalkyl” refers to an alkyl group, as defined herein, wherein at least one carbon atom has been replaced by a heteroatom selected from the group consisting of oxygen, nitrogen, or sulfur. The nitrogen atom may be substituted or unsubstituted (e.g., NR wherein R is H or other substituents, as defined). The nitrogen and sulfur heteroatoms may optionally be oxidised (i.e., N→O and S(O)ₚ, where p=1 or 2). A divalent heteroalkyl is referred to herein as “heteroalkylene.”

As used herein, the term “optionally substituted haloalkyl” refers to unsubstituted haloalkyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

As used herein, the term “alkoxy” or “alkoxyl” includes substituted and unsubstituted alkyl, alkenyl and alkynyl groups covalently linked to an oxygen atom. Examples of alkoxy groups or alkoxyl radicals include, but are not limited to, methoxy, ethoxy, isopropyloxy, propoxy, butoxy and pentoxy groups. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy and trichloromethoxy.

As used herein, the expressions “one or more of A, B, or C,” “one or more A, B, or C,” “one or more of A, B, and C,” “one or more A, B, and C,” “selected from the group consisting of A, B, and C”, “selected from A, B, and C”, and the like are used interchangeably and all refer to a selection from a group consisting of A, B, and/or C, i.e., one or more As, one or more Bs, one or more Cs, or any combination thereof, unless indicated otherwise.

It is to be understood that the present disclosure provides methods for the synthesis of the compounds of any of the Formulae described herein. The present disclosure also provides detailed methods for the synthesis of various disclosed compounds of the present disclosure according to the following schemes as well as those shown in the Examples.

It is to be understood that, throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps order for performing certain actions is immaterial so long as the invention remains operable. Moreover, two or more steps or actions can be conducted simultaneously.

It is to be understood that the synthetic processes of the disclosure can tolerate a wide variety of functional groups, therefore various substituted starting materials can be used. The processes generally provide the desired final compound at or near the end of the overall process, although it may be desirable in certain instances to further convert the compound to a pharmaceutically acceptable salt thereof.

It is to be understood that compounds of the present disclosure can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition, John Wiley & Sons: New York, 2001; Greene, T. W., Wuts, P. G. M., Protective Groups in Organic Synthesis, 3^{rd} edition, John Wiley & Sons: New York, 1999; R. Larock, Comprehensive Organic Transformations, VCH Publishers (1989); L. Fieser and M. Fieser, Fieser and Fieser's Reagents for organic Synthesis, John Wiley and Sons (1994); and L. Paquette, ed., Encyclopedia of Reagents for organic Synthesis, John Wiley and Sons (1995), incorporated by reference herein, are useful and recognised reference textbooks of organic synthesis known to those in the art

One of ordinary skill in the art will note that, during the reaction sequences and synthetic schemes described herein, the order of certain steps may be changed, such as the introduction and removal of protecting groups. One of ordinary skill in the art will recognise that certain groups may require protection from the reaction conditions via the use of protecting groups. Protecting groups may also be used to differentiate similar functional groups in molecules. A list of protecting groups and how to introduce and remove these groups can be found in Greene, T. W., Wuts, P. G. M., Protective Groups in Organic Synthesis, 3^{rd} edition, John Wiley & Sons: New York, 1999.

It is to be understood that, unless otherwise stated, any description of a method of treatment or prevention includes use of the compounds to provide such treatment or prevention as is described herein. It is to be further understood, unless otherwise stated, any description of a method of treatment or prevention includes use of the compounds to prepare a medicament to treat or prevent such condition. The treatment or prevention includes treatment or prevention of human or non-human animals including rodents and other disease models.

It is to be understood that, unless otherwise stated, any description of a method of treatment includes use of the compounds to provide such treatment as is described herein. It is to be further understood, unless otherwise stated, any description of a method of treatment includes use of the compounds to prepare a medicament to treat such condition. The treatment includes treatment of human or non-human animals including rodents and other disease models.

As used herein, the term “subject” includes human and non-human animals, as well as cell lines, cell cultures, tissues, and organs. In some embodiments, the subject is a mammal. The mammal can be e.g., a human or appropriate non-human mammal, such as primate, mouse, rat, dog, cat, cow, horse, goat, camel, sheep or a pig. The subject can also be a bird or fowl. In some embodiments, the subject is a human.

As used herein, the term “subject in need thereof” refers to a subject having a disease or having an increased risk of developing the disease. A subject in need thereof can be one who has been previously diagnosed or identified as having a disease or disorder disclosed herein. A subject in need thereof can also be one who is suffering from a disease or disorder disclosed herein. Alternatively, a subject in need thereof can be one who has an increased risk of developing such disease or disorder relative to the population at large (i.e., a subject who is predisposed to developing such disorder relative to the population at large). A subject in need thereof can have a refractory or resistant a disease or disorder disclosed herein (i.e., a disease or disorder disclosed herein that does not respond or has not yet responded to treatment). The subject may be resistant at start of treatment or may become resistant during treatment. In some embodiments, the subject in need thereof received and failed all known effective therapies for a disease or disorder disclosed herein. In some embodiments, the subject in need thereof received at least one prior therapy.

As used herein, the term “treating” or “treat” describes the management and care of a patient for the purpose of combating a disease, condition, or disorder and includes the administration of a compound of the present disclosure, or a pharmaceutically acceptable salt, polymorph or solvate thereof, to alleviate the symptoms or complications of a disease, condition or disorder, or to eliminate the disease, condition or disorder. The term “treat” can also include treatment of a cell in vitro or an animal model. It is to be appreciated that references to “treating” or “treatment” include the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

It is to be understood that a compound of the present disclosure, or a pharmaceutically acceptable salt, polymorph or solvate thereof, can or may also be used to prevent a relevant disease, condition or disorder, or used to identify suitable candidates for such purposes.

As used herein, the term “preventing,” “prevent,” or “protecting against” describes reducing or eliminating the onset of the symptoms or complications of such disease, condition or disorder.

It is to be understood that one skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3^{rd} edition), Cold Spring Harbor Press, Cold Spring Harbor, New York (2000); Coligan et al., Current Protocols in Immunology, John Wiley & Sons, N.Y.; Enna et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, PA, 18^{th} edition (1990). These texts can, of course, also be referred to in making or using an aspect of the disclosure.

It is to be understood that the present disclosure also provides pharmaceutical compositions comprising any compound described herein in combination with at least one pharmaceutically acceptable excipient or carrier.

As used herein, the term “pharmaceutical composition” is a formulation containing the compounds of the present disclosure in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In one embodiment, the active compound is mixed under sterile conditions with a pharmaceutically acceptable carrier, and with any preservatives, buffers, or propellants that are required.

As used herein, the term “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

It is to be understood that a pharmaceutical composition of the disclosure is formulated to be compatible with its intended route of administration. Examples of routes of administration include parenteral, e.g., intravenous, intradermal, subcutaneous, oral (e.g., ingestion), inhalation, transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

It is to be understood that a compound or pharmaceutical composition of the disclosure can be administered to a subject in many of the well-known methods currently used for chemotherapeutic treatment. For example, a compound of the disclosure may be injected into the blood stream or body cavities or taken orally or applied through the skin with patches. The dose chosen should be sufficient to constitute effective treatment but not so high as to cause unacceptable side effects. The state of the disease condition (e.g., a disease or disorder disclosed herein) and the health of the patient should preferably be closely monitored during and for a reasonable period after treatment.

As used herein, the term “therapeutically effective amount”, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by experimentation that is within the skill and judgment of the clinician.

It is to be understood that, for any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing active compounds of the present disclosure may be manufactured in a manner that is generally known, e.g., by means of conventional mixing, dissolving, granulating, dragee-making, levigating, emulsifying, encapsulating, entrapping, or lyophilising processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), cyclodextrins and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilisation. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, capsules or sachets. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, orange flavoring.

For administration by inhalation, the compounds are delivered in the form of an aerosol spray from pressured container or dispenser, which contains a suitable propellant, e.g., a gas such as carbon dioxide, or a nebuliser.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays, powders or suppositories. For transdermal administration, the active compounds are formulated into ointments, salves, gels, or creams as generally known in the art.

The active compounds can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It may be advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved.

In therapeutic applications, the dosages of the pharmaceutical compositions used in accordance with the disclosure vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the symptoms of the disease or disorder disclosed herein and also preferably causing complete regression of the disease or disorder. An effective amount of a pharmaceutical agent is that which provides an objectively identifiable improvement as noted by the clinician or other qualified observer. Improvement in survival and growth indicates regression. As used herein, the term “dosage effective manner” refers to amount of an active compound to produce the desired biological effect in a subject or cell.

It is to be understood that the pharmaceutical compositions can be included in a container, pack, or dispenser together with instructions for administration.

It is to be understood that, for the compounds of the present disclosure being capable of further forming salts, all of these forms are also contemplated within the scope of the claimed disclosure.

As used herein, the term “pharmaceutically acceptable salts” refer to derivatives of the compounds of the present disclosure wherein the parent compound is modified by making acid or base salts thereof. Examples of pharmaceutically acceptable salts include, but are not limited to, mineral organic acid salts of basic residues such as amines, alkali organic salts of acidic residues such as carboxylic acids, and the like. The pharmaceutically acceptable salts include the conventional non-toxic salts or the quaternary ammonium salts of the parent compound formed, for example, from non-toxic inorganic organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

In some embodiments, the pharmaceutically acceptable salt is a sodium salt, a potassium salt, a calcium salt, a magnesium salt, a diethylamine salt, a choline salt, a meglumine salt, a benzathine salt, a tromethamine salt, an ammonia salt, an arginine salt, or a lysine salt.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the compound to the cation or anion of the salt can be 1:1, or any ratio other than 1:1, e.g., 3:1, 2:1, 1:2, or 1:3.

It is to be understood that all references to pharmaceutically acceptable salts include solvent addition forms (solvates) or crystal forms (polymorphs) as defined herein, of the same salt.

The compounds, or pharmaceutically acceptable salts thereof, are administered orally, nasally, transdermally, pulmonary, inhalationally, buccally, sublingually, intraperitoneally, subcutaneously, intramuscularly, intravenously, rectally, intrapleurally, intrathecally and parenterally. In one embodiment, the compound is administered orally. One skilled in the art will recognise the advantages of certain routes of administration.

The dosage regimen utilising the compounds is selected in accordance with a variety of factors including type, species, age, weight, sex and medical condition of the patient; the severity of the condition to be treated; the route of administration; the renal and hepatic function of the patient; and the particular compound or salt thereof employed.

Techniques for formulation and administration of the disclosed compounds of the disclosure can be found in Remington: the Science and Practice of Pharmacy, 19^{th} edition, Mack Publishing Co., Easton, PA (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous organic solutions. The compounds will be present in such pharmaceutical compositions in amounts sufficient to provide the desired dosage amount in the range described herein.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

In the synthetic schemes described herein, compounds may be drawn with one particular configuration for simplicity. Such particular configurations are not to be construed as limiting the disclosure to one or another isomer, tautomer, regioisomer or stereoisomer, nor does it exclude mixtures of isomers, tautomers, regioisomers or stereoisomers; however, it will be understood that a given isomer, tautomer, regioisomer or stereoisomer may have a higher level of activity than another isomer, tautomer, regioisomer or stereoisomer.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

As use herein, the phrase “compound of the disclosure” refers to those compounds which are disclosed herein, both generically and specifically.

### Compounds of the Present Disclosure

In an aspect, the present disclosure provides a compound of Formula (I**):

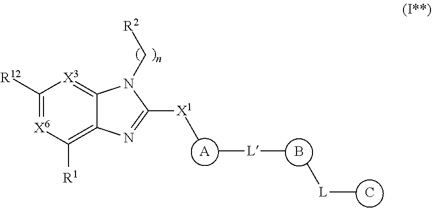

or a pharmaceutically acceptable salt thereof; wherein:
X³ is CR⁶ or N;
X⁶ is CR⁴ or N;
R¹ is —C₁₋₆ haloalkyl, halogen, —O—X⁴, or —NR⁸R⁹, or R¹ and R⁴, together with the atoms to which they are attached, combine to form a 5- or 6-membered heterocyclyl;
X⁴ is hydrogen, C₁₋₆ alkyl, C₁₋₆ heteroalkyl, C₁₋₆ haloalkyl, —(C₁₋₆ alkyl)-(C₃₋₁₀ cycloalkyl), or —(C₁₋₆ alkyl)-(3- to 8-membered heterocyclyl), 3- to 8-membered heterocyclyl, —(CH₂CH₂—O)₁₋₅—CH₃, —(CH₂—CH(—OCH₃)—CH₂—O)₁₋₅—CH₃, C₃₋₁₀ cycloalkyl, or C₆₋₁₀ aryl, wherein the alkyl, heteroalkyl, or haloalkyl group is optionally substituted with one or more deuterium, C₁₋₆ alkoxy, hydroxyl, —CN, or oxo, and the cycloalkyl, heterocyclyl, or aryl group is optionally substituted with one or more halogen, C₁₋₆ alkoxy, or —CN;
R⁶ is hydrogen, halogen, or —O—R⁷;
   wherein R⁷ and R², together with the atoms to which they are attached, combine to form a 6-membered heterocyclyl;
R⁸ and R⁹ each independently are selected from hydrogen, C₁₋₆ alkyl, or —S(O)₂—C₁₋₆ alkyl, or R⁸ and R⁹, together with the atoms to which they are attached, combine to form a 6-membered heterocycyl; wherein the C₁₋₆ alkyl is optionally substituted by one or more oxo;
n is 0, 1, 2, 3, 4, 5, or 6;
R² is:
   hydrogen;
   C₁₋₆ alkyl optionally substituted with deuterium;
   C₁₋₆ haloalkyl;
   —(O)—C₁₋₆ alkyl;
   —CN;
   a C₃₋₁₀ cycloalkyl optionally substituted with one or more —CN, C₁₋₆ haloalkyl, or C₁₋₆ alkyl optionally substituted with one or more —CN;
   a 4 or 5-membered heterocyclyl comprising at least one oxygen or at least one sulfur atom, wherein the 4- or 5-membered heterocyclyl is optionally substituted with one or more oxo,

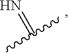 [SMILES: *=N]

or C₁₋₆ alkyl;
a 5-membered heteroaryl, comprising 1 or 2 heteroatoms independently selected from N, and S, wherein at least one heteroatom of R⁵ is S; or
R² and R⁷, together with the atoms to which they are attached, combine to form a 5- or 6-membered heterocyclyl;
R⁴ is halogen, hydrogen, —C(O)OH, or —O—R⁸,
   wherein R⁸ and R¹, together with the atoms to which they are attached, combine to form a 6-membered heterocyclyl;
R¹² is hydrogen, —C(O)OH, —C(O)NR^{N12}R^{N12′}, —C(O)NR¹²S(O)₂R^{12′}, —(C₂₋₆ alkynylene)-C(O)OH, —(C₁₋₆ alkylene)-C(O)OH, —NR^{N12}—(C₁₋₆ alkylene)-C(O)OH, 5-10 membered heteroaryl or 5- to 10-membered heterocyclyl optionally substituted with one or more oxo, C₁₋₆ alkyl or C₁₋₆ haloalkyl;
R^{N12} and R^{N12′} independently are H or C₁₋₆ alkyl;

X¹ is

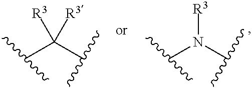

wherein R³ and R^{3′} independently are H, D or C₁₋₆ alkyl, wherein the C₁₋₆ alkyl is optionally substituted with deuterium;
Ring A is

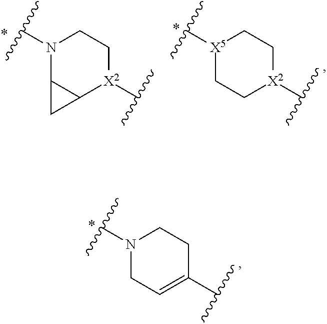

phenyl optionally substituted with one or more halo or C₁₋₆ alkyl, or 6-membered heteroaryl optionally substituted with one or more halo or C₁₋₆ alkyl;
wherein * indicates attachment to X¹,
   X⁵ is CR³ or N, and
   X² is CR³ or N;
L′ is a bond or —O—;
Ring B is a C₆₋₁₀ arylene, a 5-10 membered heteroarylene, or a 3-10 membered heterocycylene, wherein the C₆₋₁₀ arylene, 5-10 membered heteroarylene, or 3-10 membered heterocyclene is optionally substituted with one or more oxo, C₁₋₆ alkyl, C₁₋₆ alkoxy, or halogen;
L is a bond, *—(C₁₋₆ alkylene)-, *—NR^{L}—(C₁₋₆ alkyl), *—O—(C₁₋₆ alkyl)-, or *—(C₁₋₆ alkyl)-O—, wherein * indicates attachment to Ring B and the C₁₋₆ alkylene or C₁₋₆ alkyl is optionally substituted with deuterium;
   wherein R^{L} is H or C₁₋₆ alkyl; and
Ring C is:
a 6-membered aryl optionally substituted with one or more C₃₋₁₀ cycloalkyl, C₁₋₆ alkyl, C₁₋₆ haloalkyl, 3-10 membered heterocyclyl, halogen, C₁₋₆ alkoxy, C₁₋₆ haloalkoxy, —CN, C₃₋₁₀ cycloalkyl, or —C(O)NR′₂;
   wherein R′ is H or C₁₋₆ alkyl
a 6-membered heteroaryl comprising a nitrogen atom optionally substituted with one or more halogen, —CN, C₁₋₆ haloalkyl, —O—C₁₋₆ alkyl, C₃₋₁₀ cycloalkyl, —C(═O)—(C₃₋₁₀ cycloalkyl),

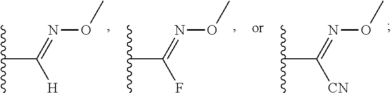

or

a bicylic 9- or 10-membered heteroaryl or heterocyclyl optionally substituted with one or more C₁₋₆ alkyl, halogen, —CN, or oxo.

In an aspect, the present disclosure provides a compound of Formula (I**-4):

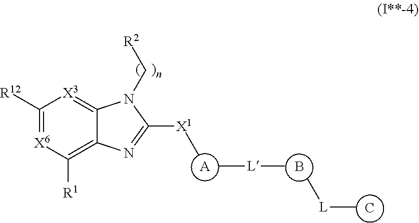

or a pharmaceutically acceptable salt thereof; wherein:
X³ is CR⁶ or N;
X⁶ is CR⁴ or N;
R¹ is —C₁₋₆ haloalkyl, halogen, —O—X⁴, or —NR⁸R⁹, or R¹ and R⁴, together with the atoms to which they are attached, combine to form a 6-membered heterocyclyl;
X⁴ is hydrogen, C₁₋₆ alkyl, C₁₋₆ heteroalkyl, C₁₋₆ haloalkyl, —(C₁₋₆ alkyl)-(C₃₋₁₀ cycloalkyl), or —(C₁₋₆ alkyl)-(3- to 8-membered heterocyclyl), 3- to 8-membered heterocyclyl, —(CH₂CH₂—O)₁₋₅—CH₃, or —(CH₂—CH(—OCH₃)—CH₂—O)₁₋₅—CH₃, wherein the alkyl, heteroalkyl, or haloalkyl group is optionally substituted with one or more deuterium, C₁₋₆ alkoxy, hydroxyl, —CN, or oxo, and the cycloalkyl or heterocyclyl group is optionally substituted with one or more halogen or —CN;
R⁶ is hydrogen, halogen, or —O—R⁷;
   wherein R⁷ and R², together with the atoms to which they are attached, combine to form a 6-membered heterocyclyl;
R⁸ and R⁹ each independently are selected from hydrogen, C₁₋₆ alkyl, or —S(O)₂—C₁₋₆ alkyl, or R⁸ and R⁹, together with the atoms to which they are attached, combine to form a 6-membered heterocycyl; wherein the C₁₋₆ alkyl is optionally substituted by one or more oxo;
n is 0, 1, 2, 3, 4, 5, or 6;
R² is:
   hydrogen;
   C₁₋₆ alkyl optionally substituted with deuterium;
   C₁₋₆ haloalkyl;
   —(O)—C₁₋₆ alkyl;
   —CN;
   a C₃₋₁₀ cycloalkyl optionally substituted with one or more —CN, C₁₋₆ haloalkyl, or C₁₋₆ alkyl optionally substituted with one or more —CN;
   a 4 or 5-membered heterocyclyl comprising at least one oxygen or at least one sulfur atom, wherein the 4- or 5-membered heterocyclyl is optionally substituted with one or more oxo,

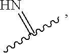 [SMILES: *=N]

or C₁₋₆ alkyl;
a 5-membered heteroaryl, comprising 1 or 2 heteroatoms independently selected from N, and S, wherein at least one heteroatom of R⁵ is S; or
R² and R⁷, together with the atoms to which they are attached, combine to form a 5- or 6-membered heterocyclyl;
R⁴ is hydrogen, —C(O)OH, or —O—R⁸,
   wherein R⁸ and R¹, together with the atoms to which they are attached, combine to form a 6-membered heterocyclyl;
R¹² is hydrogen, —C(O)OH or 5-10 membered heteroaryl optionally substituted with one or more oxo, C₁₋₆ alkyl or C₁₋₆ haloalkyl;
X¹ is

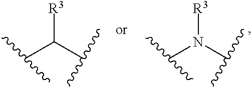

wherein R³ is H or C₁₋₆ alkyl;
Ring A is

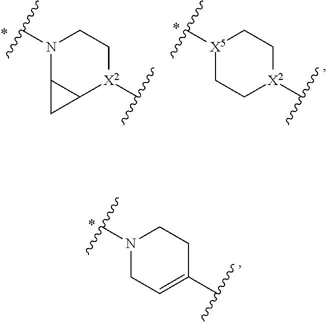

phenyl optionally substituted with one or more halo or C₁₋₆ alkyl, or 6-membered heteroaryl optionally substituted with one or more halo or C₁₋₆ alkyl;
wherein * indicates attachment to X¹,
   X⁵ is CR³ or N, and
   X² is CR³ or N;
L′ is a bond or —O—;
Ring B is a C₆₋₁₀ arylene, a 5-10 membered heteroarylene, or a 3-10 membered heterocycylene, wherein the C₆₋₁₀ arylene, 5-10 membered heteroarylene, or 3-10 membered heterocyclene is optionally substituted with one or more oxo, C₁₋₆ alkyl, C₁₋₆ alkoxy, or halogen;
L is a bond, *—(C₁₋₆ alkylene)-, *—NR^{L}—(C₁₋₆ alkyl), *—O—(C₁₋₆ alkyl)-, or *—(C₁₋₆ alkyl)-O—, wherein * indicates attachment to Ring B and the C₁₋₆ alkylene or C₁₋₆ alkyl is optionally substituted with deuterium;
   wherein R^{L} is H or C₁₋₆ alkyl; and
Ring C is:
a 6-membered aryl optionally substituted with one or more C₁₋₆ alkyl, C₁₋₆ haloalkyl, 3-10 membered heterocyclyl, halogen, C₁₋₆ alkoxy, C₁₋₆ haloalkoxy, —CN, C₃₋₁₀ cycloalkyl, or —C(O)NR′₂;
   wherein R′ is H or C₁₋₆ alkyl
a 6-membered heteroaryl comprising a nitrogen atom optionally substituted with one or more halogen, —CN, C₁₋₆ haloalkyl, —O—C₁₋₆ alkyl, C₃₋₁₀ cycloalkyl, —C(═O)—(C₃₋₁₀ cycloalkyl),

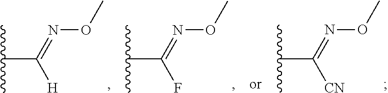

or
   a bicylic 9- or 10-membered heteroaryl optionally substituted with one or more C₁₋₆ alkyl, halogen, or oxo.

In an aspect, the present disclosure provides a compound of Formula (I):

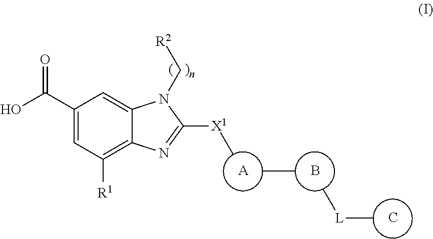

or a pharmaceutically acceptable salt thereof, wherein
R¹ is —C₁₋₆ haloalkyl or —O—X⁴;
wherein X⁴ is C₁₋₆ alkyl, C₁₋₆ heteroalkyl, C₁₋₆ haloalkyl, —(C₁₋₆ alkyl)-(C₃₋₁₀ cycloalkyl), or
(C₁₋₆ alkyl)-(3- to 8-membered heterocyclyl), or —CH₂CH₂—(—O—CH₂CH₂—)₁₋₅—CH₃, wherein the alkyl, heteroalkyl, or haloalkyl group is optionally substituted with one or more —CN, and the cycloalkyl or heterocyclyl group is optionally substituted with one or more halogen or —CN;
n is 0, 1, 2, 3, 4, 5, or 6;
R² is:
a C₃₋₁₀ cycloalkyl optionally substituted with one or more —CN, C₁₋₆ haloalkyl, or C₁₋₆ alkyl optionally substituted with one or more —CN;
a 4 or 5-membered heterocyclyl comprising at least one oxygen or at least one sulfur atom, wherein the 4- or 5-membered heterocyclyl is optionally substituted with one or more oxo or C₁₋₆ alkyl; or
a 5-membered heteroaryl, comprising 1 or 2 heteroatoms independently selected from N, and S, wherein at least one heteroatom of R⁵ is S;
X¹ is

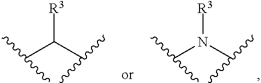

wherein R³ is H or C₁₋₆ alkyl;
Ring A is

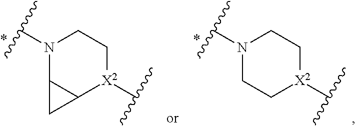

wherein * indicates attachment to X¹, and
X² is CH or N;
Ring B is:
a 6-membered heteroarylene comprising nitrogen;
a 9-membered heterocycylene comprising two oxygen atoms optionally substituted with one or more C₁₋₆ alkyl; or a 10-membered heterocycylene comprising two oxygen atoms;
L is a bond or *—O—(C₁₋₆ alkyl)-, wherein * indicates attachment to Ring B;
Ring C is:
a 6-membered aryl optionally substituted with one or more halogen, —OCH₃, —CN, or C₃₋₁₀ cycloalkyl;
a 6-membered heteroaryl comprising a nitrogen atom optionally substituted with one or more halogen, —CN, —O—C₁₋₆ alkyl, C₃₋₁₀ cycloalkyl,

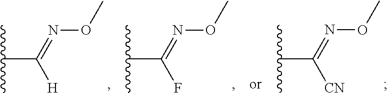

a bicylic 9- or 10-membered heteroaryl comprising two nitrogen atoms optionally substituted with one or more C₁₋₆ alkyl, or oxo.

In some embodiments, the compound of Formula (I**) is of Formula (I*):

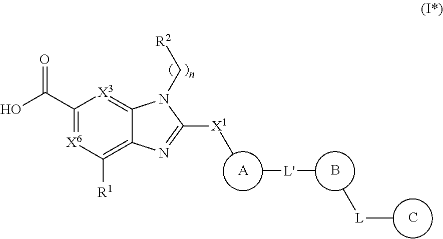

or a pharmaceutically acceptable salt thereof; wherein:
X³ is CR⁶ or N;
X⁶ is CR⁴ or N;
R¹ is —C₁₋₆ haloalkyl, —O—X⁴, or —NR⁸R⁹, or R¹ and R⁴, together with the atoms to which they are attached, combine to form a 6-membered heterocyclyl;
X⁴ is hydrogen, C₁₋₆ alkyl, C₁₋₆ heteroalkyl, C₁₋₆ haloalkyl, —(C₁₋₆ alkyl)-(C₃₋₁₀ cycloalkyl), or —(C₁₋₆ alkyl)-(3- to 8-membered heterocyclyl), 3- to 8-membered heterocyclyl, —(CH₂CH₂—O)₁₋₅—CH₃, or —(CH₂—CH(—OCH₃)—CH₂—O)₁₋₅—CH₃, wherein the alkyl, heteroalkyl, or haloalkyl group is optionally substituted with one or more deuterium, hydroxyl, —CN, or oxo, and the cycloalkyl or heterocyclyl group is optionally substituted with one or more halogen or —CN;
R⁶ is hydrogen, halogen, or —O—R⁷;
   wherein R⁷ and R², together with the atoms to which they are attached, combine to form a 6-membered heterocyclyl;
R⁸ and R⁹ each independently are selected from hydrogen, C₁₋₆ alkyl, or —S(O)₂—C₁₋₆ alkyl, or R⁸ and R⁹, together with the atoms to which they are attached, combine to form a 6-membered heterocycyl; wherein the C₁₋₆ alkyl is optionally substituted by one or more oxo;
n is 0, 1, 2, 3, 4, 5, or 6;
R² is:
hydrogen;
C₁₋₆ alkyl;
C₁₋₆ haloalkyl;
—(O)—C₁₋₆ alkyl;
a C₃₋₁₀ cycloalkyl optionally substituted with one or more —CN, C₁₋₆ haloalkyl, or C₁₋₆ alkyl optionally substituted with one or more —CN;
a 4 or 5-membered heterocyclyl comprising at least one oxygen or at least one sulfur atom, wherein the 4- or 5-membered heterocyclyl is optionally substituted with one or more oxo,

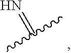 [SMILES: *=N]

or C₁₋₆ alkyl;
a 5-membered heteroaryl, comprising 1 or 2 heteroatoms independently selected from N, and S, wherein at least one heteroatom of R⁵ is S; or
R² and R⁷, together with the atoms to which they are attached, combine to form a 5- or 6-membered heterocyclyl;
R⁴ is hydrogen or —O—R⁸,
   wherein R⁸ and R¹, together with the atoms to which they are attached, combine to form a 6-membered heterocyclyl;
X¹ is

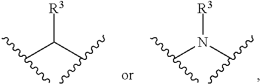

wherein R³ is H or C₁₋₆ alkyl;
Ring A is

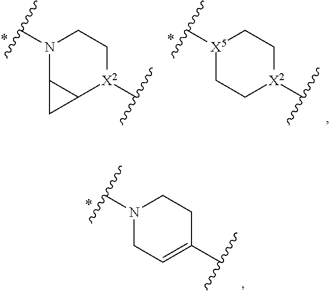

phenyl optionally substituted with one or more halo or C₁₋₆ alkyl, or 6-membered heteroaryl optionally substituted with one or more halo or C₁₋₆ alkyl;
wherein * indicates attachment to X¹,
   X⁵ is CR³ or N, and
   X² is CR³ or N;
L′ is a bond or —O—;
Ring B is a C₆₋₁₀ arylene, a 6-10 membered heteroarylene, or a 3-10 membered heterocycylene, wherein the C₆₋₁₀ arylene, 6-10 heteroarylene, or 3-10 membered heterocyclene is optionally substituted with one or more C₁₋₆ alkyl, C₁₋₆ alkoxy, or halogen;
L is a bond, *—CH₂—, *—O—(C₁₋₆ alkyl)-, or *—(C₁₋₆ alkyl)-O—, wherein * indicates attachment to Ring B and the C₁₋₆ alkyl is optionally substituted with deuterium; and
Ring C is:
a 6-membered aryl optionally substituted with one or more C₁₋₆ alkyl, 3-10 membered heterocyclyl, halogen, —OCH₃, —CN, or C₃₋₁₀ cycloalkyl;
a 6-membered heteroaryl comprising a nitrogen atom optionally substituted with one or more halogen, —CN, —O—C₁₋₆ alkyl, C₃₋₁₀ cycloalkyl, —C(═O)—(C₃₋₁₀ cycloalkyl),

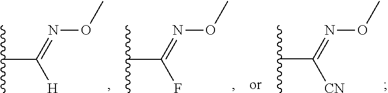

or
a bicylic 9- or 10-membered heteroaryl comprising two nitrogen atoms optionally substituted with one or more C₁₋₆ alkyl, or oxo.

In an aspect, the present disclosure provides a compound of Formula I′″*:

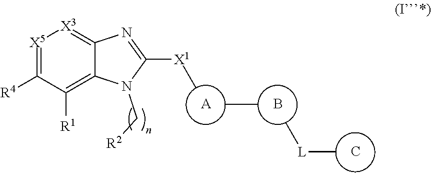

or a pharmaceutically acceptable salt thereof; wherein:
X³ is CR^{X3} or N;
   wherein R^{X3} is H, halogen, C₁₋₆ alkyl, C₁₋₆ haloalkoxy, —O—(CH₂CH₂—O)₁₋₅—CH₃, or C₁₋₆ alkoxy;
X^{5′} is CR^{5′}
R¹ is —C₁₋₆ haloalkyl or —O—X⁴, or R¹ and R⁴, together with the atoms to which they are attached, combine to form a 6-membered heterocyclyl;
wherein X⁴ is hydrogen, C₁₋₆ alkyl, C₁₋₆ heteroalkyl, C₁₋₆ haloalkyl, —(C₁₋₆ alkyl)-(C₃₋₁₀ cycloalkyl), or —(C₁₋₆ alkyl)-(3- to 8-membered heterocyclyl), 3- to 8-membered heterocyclyl, —(CH₂CH₂—O)₁₋₅—CH₃, or —(CH₂—CH(—OCH₃)—CH₂—O)₁₋₅—CH₃, wherein the alkyl, heteroalkyl, or haloalkyl group is optionally substituted with one or more deuterium, halogen, or —CN, and the cycloalkyl or heterocyclyl group is optionally substituted with one or more halogen or —CN;
n is 0, 1, 2, 3, 4, 5, or 6;
R² is:
   hydrogen;
   halogen;
   branched C₃₋₆ alkyl;
   —(O)—C₁₋₆ alkyl;
   C₃₋₁₀ cycloalkyl optionally substituted with one or more —CN, C₁₋₆ haloalkyl, or C₁₋₆ alkyl optionally substituted with one or more —CN;
   4 or 5-membered heterocyclyl comprising at least one oxygen or at least one sulfur atom, wherein the 4- or 5-membered heterocyclyl is optionally substituted with one or more oxo,

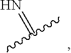 [SMILES: *=N]

or C₁₋₆ alkyl; or
5-membered heteroaryl, comprising 1 or 2 heteroatoms independently selected from N, and S, wherein at least one heteroatom of R⁵ is S; or
R² and R¹, together with the atoms to which they are attached, combine to form a 6-membered heterocyclyl;
R^{5′} is hydrogen or —C(O)—OH;
R⁴ is hydrogen, halogen, —C(O)—OH, or —O—R⁸,
   wherein R⁸ and R¹, together with the atoms to which they are attached, combine to form a 6-membered heterocyclyl; X¹ is

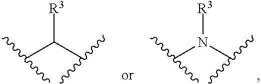

wherein R³ is H or C₁₋₆ alkyl;

### Ring A is

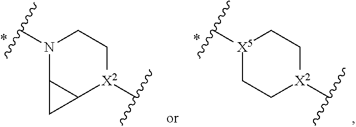 [0652] wherein * indicates attachment to X¹,
wherein X⁵ is CH or N, and
   X² is CH or N;
Ring B is a 6-10 membered heteroarylene, or a 3-10 membered heterocycylene optionally substituted with one or more C₁₋₆ alkyl;
L is a bond or *—O—(C₁₋₆ alkyl)-, wherein * indicates attachment to Ring B;
Ring C is:
a 6-membered aryl optionally substituted with one or more halogen, —OCH₃, —CN, or C₃₋₁₀ cycloalkyl;
a 6-membered heteroaryl comprising a nitrogen atom optionally substituted with one or more halogen, —CN, —O—C₁₋₆ alkyl, C₃₋₁₀ cycloalkyl, —C(═O)—(C₃₋₁₀ cycloalkyl),

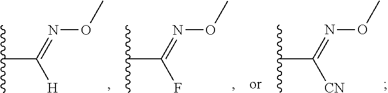

or
a bicylic 9- or 10-membered heteroaryl comprising two nitrogen atoms optionally substituted with one or more C₁₋₆ alkyl, or oxo.

In some embodiments, the compound of Formula (I**) is of Formula (II**):

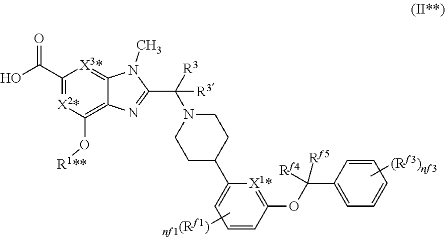

or a pharmaceutically acceptable salt thereof; wherein
R^{f4} and R^{f5} are each independently selected from C₁₋₆ alkyl, H and D
nf1 is 0, 1, 2, 3, or 4;
nf3 is 0, 1, 2, 3, 4, or 5;
each R^{f1} is halogen;
R³ and R^{3′} independently are H or D;
X¹* is N or CR^{f1};
X^{2*} and X^{3*} independently are CH or CF;
each R^{f3} is independently selected from halogen, —CN, C₁₋₆ alkyl, C₁₋₆ haloalkyl, C₁₋₆ alkoxy, or —C(O)N(R^{f3′})₂,
each R^{f3′} is independently selected from H or C₁₋₆ alkyl; and
R¹** is H or C₁₋₂ alkyl optionally substituted with one or more deuterium or halogen.

In some embodiments, the compound of Formula (I**) is of Formula (II*)

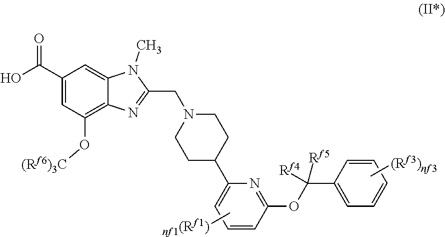

or a pharmaceutically acceptable salt thereof;
each R^{f1} is independently selected from halogen
R^{f4} and R^{f5} are each independently selected from C₁₋₆ alkyl, H and D
nf1 is 0, 1, 2, or 3;
nf3 is 0, 1, 2, 3, 4, or 5;
each R^{f1} is halogen;
each R^{f3} is independently selected from halogen, —CN, C₁₋₆ alkyl, C₁₋₆ haloalkyl, C₁₋₆ alkoxy, or —C(O)N(R^{f3′})₂,
   each R^{f3′} is independently selected from H or C₁₋₆ alkyl;
each R^{f6} is independently selected from H, D or F.

In some embodiments, R^{f4} and R^{f5} are each independently selected from CH₃, H, and D.

In some embodiments, each R^{f1} is fluorine.

In some embodiments the compound of Formula (I**) is of Formula (IA):

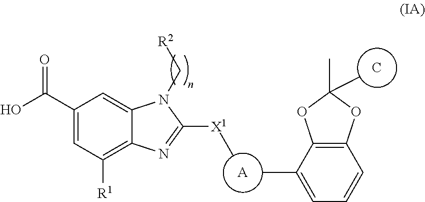

or a pharmaceutically acceptable salt thereof;
wherein R¹, n, R², X¹, Ring A, and Ring C are as defined for Formula (I**).

In some embodiments, the compound of Formula (I**) is of Formula (IB):

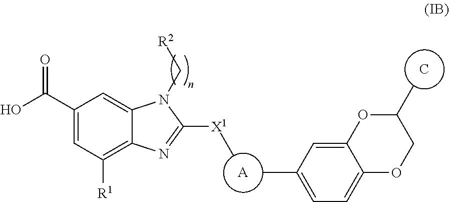

or a pharmaceutically acceptable salt thereof;
wherein R¹, n, R², X¹, Ring A, and Ring C are as defined for Formula (I**).

In some embodiments, the compound of Formula (I**) is of Formula (IC):

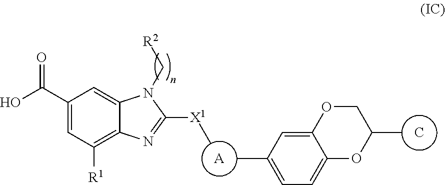

or a pharmaceutically acceptable salt thereof;
wherein R¹, n, R², X¹, Ring A, and Ring C are as defined for Formula (I**).

In some embodiments, the compound of Formula (I**) is of Formula (ID):

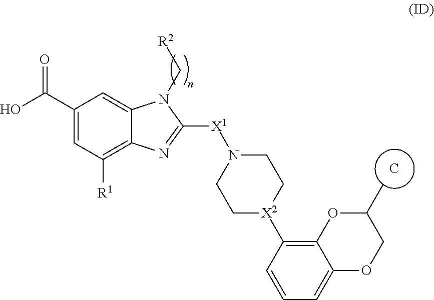

or a pharmaceutically acceptable salt thereof;
wherein R¹, n, R², X¹, X², and Ring C are as defined for Formula (I**).

In some embodiments, the compound of Formula (I**) is of Formula (IE):

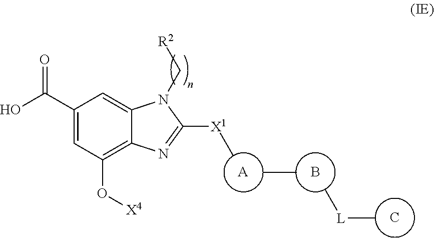

or a pharmaceutically acceptable salt thereof;
wherein X⁴, n, R², X¹, Ring A, Ring B, and Ring C are as defined for Formula (I**).

In some embodiments, the compound of Formula (I**) is of Formula (IF):

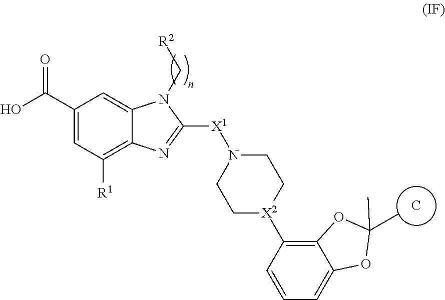

or a pharmaceutically acceptable salt thereof;
wherein R¹, n, R², X¹, and Ring C are as defined for Formula (I**).

In some embodiments, the compound of Formula (I**) is of Formula (IG):

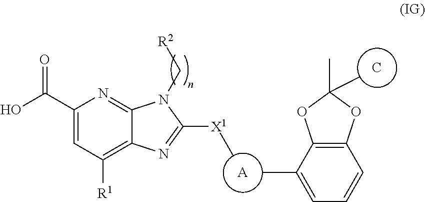

or a pharmaceutically acceptable salt thereof;
wherein R¹, n, R², X¹, Ring A, and Ring C are as defined for Formula (I**).

In some embodiments, the compound of Formula (I**) is of Formula (IH):

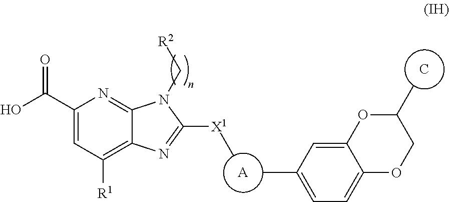

or a pharmaceutically acceptable salt thereof;
wherein R¹, n, R², X¹, Ring A, and Ring C are as defined for Formula (I**).

In some embodiments, the compound of Formula (I**) is of Formula (II):

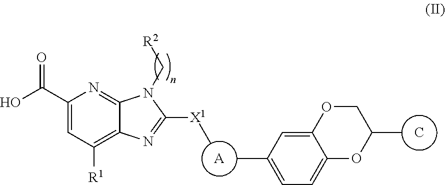

or a pharmaceutically acceptable salt thereof;
wherein R¹, n, R², X¹, and Ring A, Ring C are as defined for Formula (I**).

In some embodiments, the compound of Formula (I**) is of Formula (IJ)

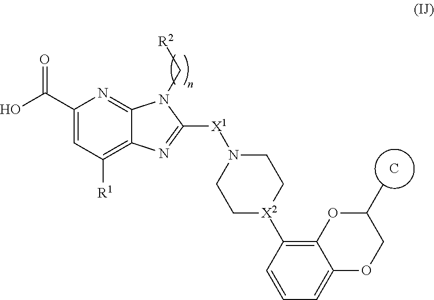

or a pharmaceutically acceptable salt thereof;
wherein R¹, n, R², X¹, and Ring C are as defined for Formula (I**).

In some embodiments, the compound of Formula (I**) is of Formula (IK)

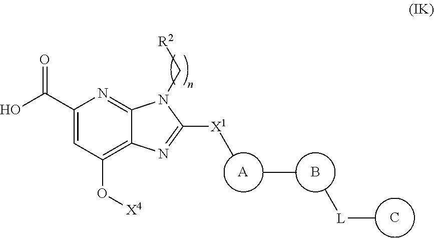

or a pharmaceutically acceptable salt thereof;
wherein X⁴, R², X¹, n Ring A, Ring B, L, and Ring C are as defined for Formula (I**).

In some embodiments, the compound of Formula (I**) is of Formula (IL)

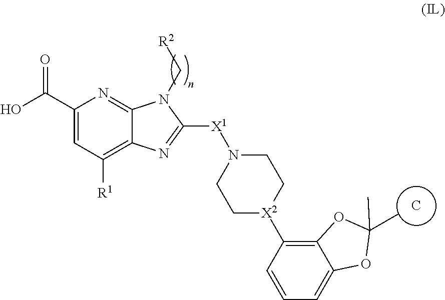

or a pharmaceutically acceptable salt thereof;
wherein R¹, R², X¹, n, and Ring C are as defined for Formula (I**).

In some embodiments, the compound of Formula (I**) is of Formula (IM)

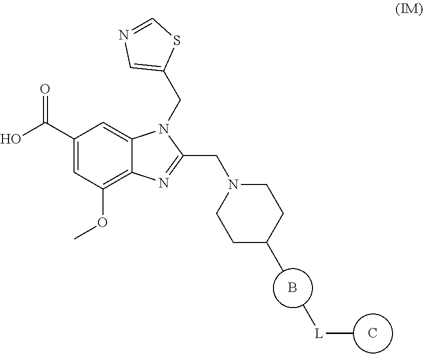

or a pharmaceutically acceptable salt thereof;
wherein Ring B, L, and Ring C are as defined for Formula (I**).

In some embodiments, the compound of Formula (I**) is of Formula (IN)

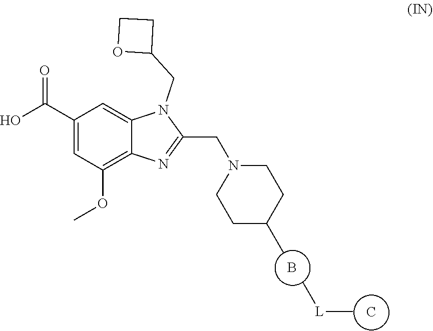

or a pharmaceutically acceptable salt thereof;
wherein Ring B, L, and Ring C are as defined for Formula (I**).

In some embodiments, the compound of Formula (I**) is of Formula (IO)

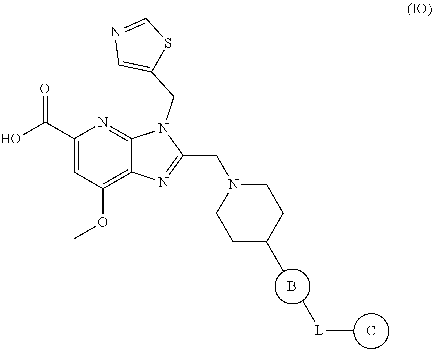

or a pharmaceutically acceptable salt thereof;
wherein Ring B, L, and Ring C are as defined for Formula (I**).

In some embodiments, the compound of Formula (I**) is of Formula (IP)

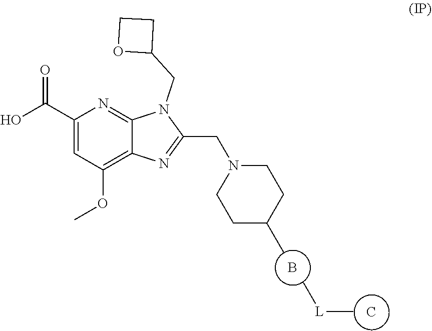

or a pharmaceutically acceptable salt thereof;
wherein Ring B, L, and Ring C are as defined for Formula (I**).

In some embodiments, the compound of Formula (I**) is of Formula (IQ)

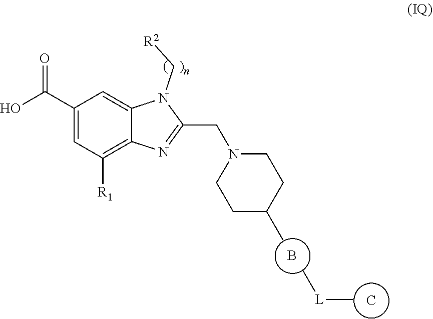

or a pharmaceutically acceptable salt thereof;
wherein R¹, R², n, Ring B, L, and Ring C are as defined for Formula (I**).

In some embodiments, the compound of Formula (I**) is of Formula (IR)

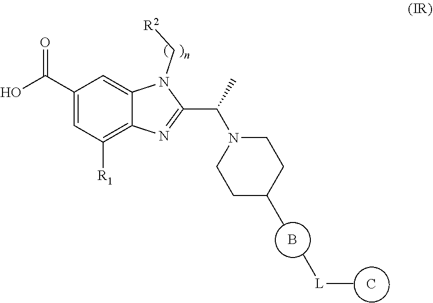

or a pharmaceutically acceptable salt thereof;
wherein R¹, R², n, Ring B, L, and Ring C are as defined for Formula (I**).

In some embodiments, the compound of Formula (I**) is of Formula (IS)

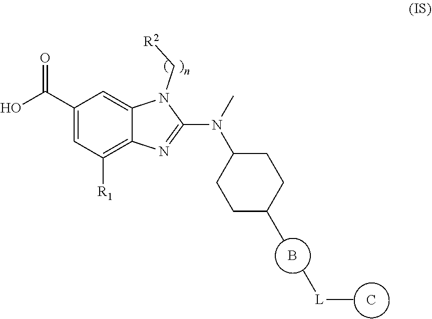

Wherein R¹, R², n, Ring B, L, and Ring C are as defined for Formula (I**).

In some embodiments, the compound of Formula (I**) is of Formula (IT)

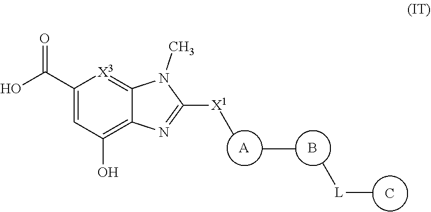

or a pharmaceutically acceptable salt thereof;
wherein X¹, X³, Ring A, Ring B, L, and Ring C are as defined for Formula (I**).

In some embodiments, the compound of Formula (I**) is of Formula (IU)

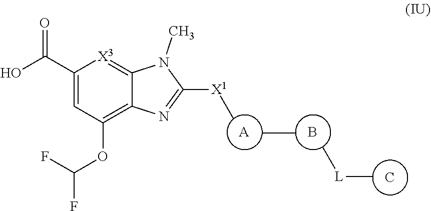

or a pharmaceutically acceptable salt thereof;
wherein X³, X¹, Ring A, Ring B, L, and Ring C are as defined for Formula (I**).
or a pharmaceutically acceptable salt thereof;
   wherein X³, R⁴, R¹, n, and R² are as defined for Formula (I**).

In some embodiments, the compound of Formula (I**) is of Formula (IX)

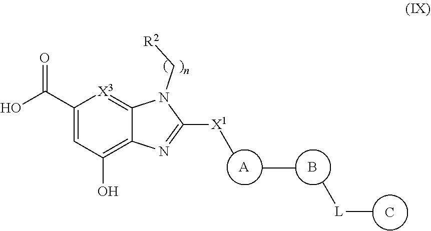

or a pharmaceutically acceptable salt thereof;
wherein X³, n, R², X¹, Ring A, Ring B, L, and Ring C are as defined for Formula (I**).

In some embodiments, the compound of Formula (I**) is of Formula (IY)

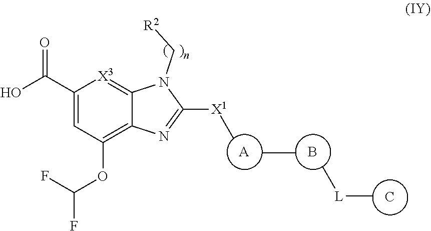

or a pharmaceutically acceptable salt thereof;
wherein X³, n, R², X¹, Ring A, Ring B, L, and Ring C are as defined for Formula (I**).

In some embodiments, the compound of Formula (I**) is of Formula (IZ)

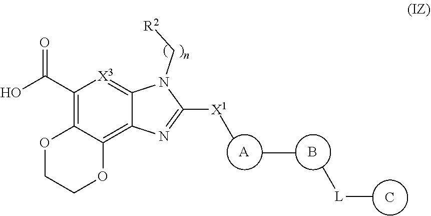

or a pharmaceutically acceptable salt thereof;
wherein X³, n, R², X¹, Ring A, Ring B, L, and Ring C are as defined for Formula (I**).

In some embodiments, the compound of Formula (I′″) is of Formula (IAA)

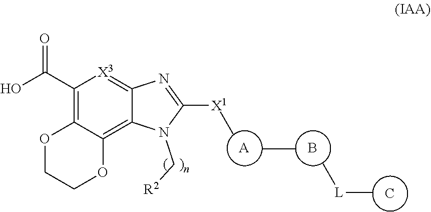

or a pharmaceutically acceptable salt thereof;
wherein X³, n, R², X¹, Ring A, Ring B, L, and Ring C are as defined for Formula (I*).

In some embodiments, the compound of Formula (I**) is of Formula (IAB)

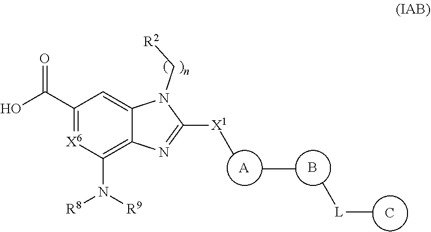

or a pharmaceutically acceptable salt thereof;
wherein X6, R8, R9, n, R2, X1, Ring A, Ring B, L, and Ring C are as defined for Formula (I**).

In some embodiments, the compound of Formula (I**) is of Formula (IAC)

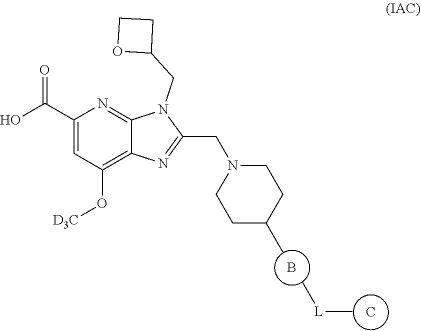

or a pharmaceutically acceptable salt thereof;
wherein Ring B, L, and Ring C are as defined for Formula (I**).

In some embodiments, the compound of Formula (I**) is of Formula (IAD)

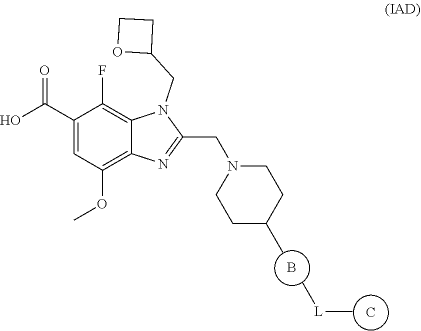

or a pharmaceutically acceptable salt thereof;
wherein Ring B, L, and Ring C are as defined for Formula (I**).

In some embodiments, the compound of Formula (I**) is of Formula (IAE)

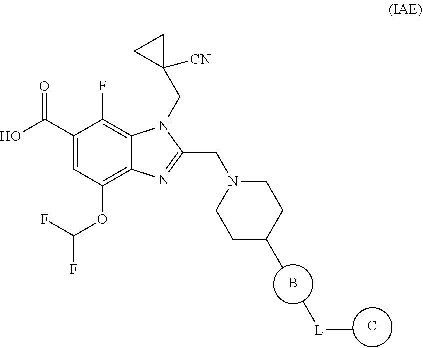

or a pharmaceutically acceptable salt thereof;
wherein Ring B, L, and Ring C are as defined for Formula (I**).

In some embodiments, the compound of Formula (I**) is of Formula (IAG)

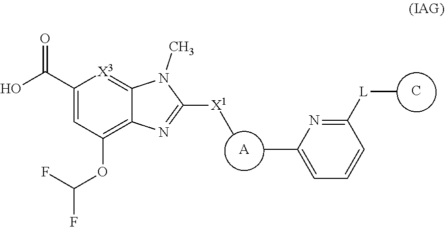

or a pharmaceutically acceptable salt thereof;
wherein X3, X1, Ring A, L, and Ring C are as defined for Formula (I**).

In some embodiments, the compound of Formula (I**) is of Formula (IAH):

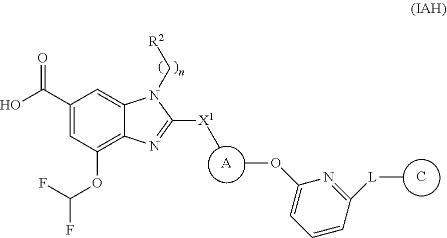

or a pharmaceutically acceptable salt thereof;
wherein n, R2, X1, Ring A, L, and Ring C are as defined for Formula (I**).

In some embodiments, the compound of Formula (I**) is of Formula (IAI):

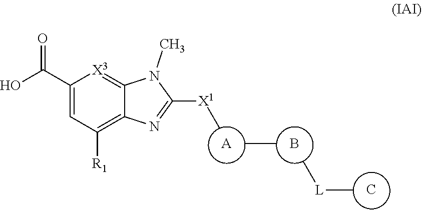

wherein R¹ is —O—(C₁₋₆ haloalkyl);
X3, X1, Ring A, Ring B, L, and Ring C are as defined for Formula (I**);
or a pharmaceutically acceptable salt thereof.

In some embodiments, the compound of Formula (I**) is of Formula (IAJ):

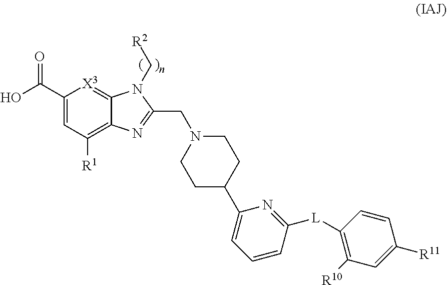

wherein R² is hydrogen, thiazolyl, oxetanyl, cyclopropyl optionally substituted with cyano, or methoxy;
n is 1 or 2;

R¹ is —O—C₁₋₆ alkyl, —OH, or —NH₂, wherein the C₁₋₆ alkyl is optionally substituted with one or more halo or deuterium;
X³ is CR⁶, wherein R⁶ is halo or hydrogen;
L is *—O—(C₁₋₆ alkyl)-, wherein * indicates attachment to Ring B and the C₁₋₆ alkyl is optionally substituted with deuterium;
R¹⁰ is halo; and
R¹¹ is halo or cyano;
or a pharmaceutically acceptable salt thereof.

The present disclosure contemplates the combination of any one of Formula (I**), (II*), (I*), (I″), (I′″), (I′), (I), (IE), (IK), (IL), (IM), (IN), (IO), (IP) (IQ), (IR), (IS), (IT), (IU), (IX), (IY), (IZ), (IAA), (IAB), (IAC), (IAD), (IAE), or (IAI) with any one of the following

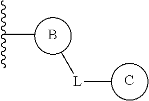

moieties:

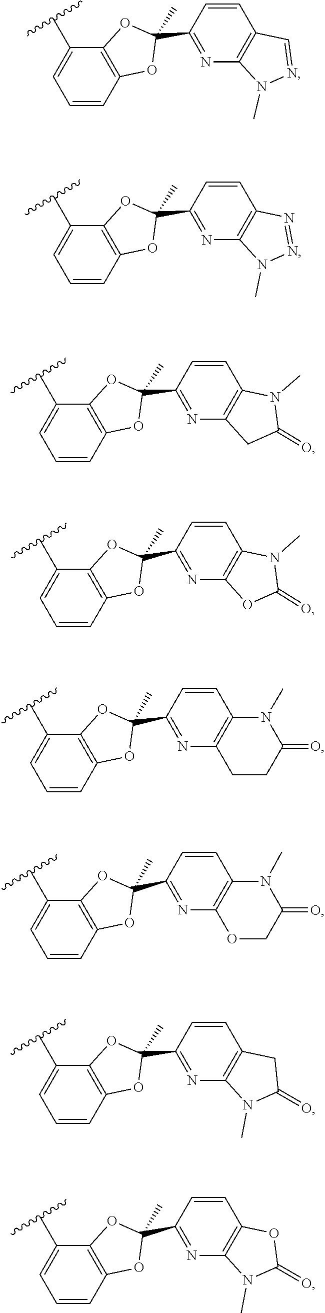

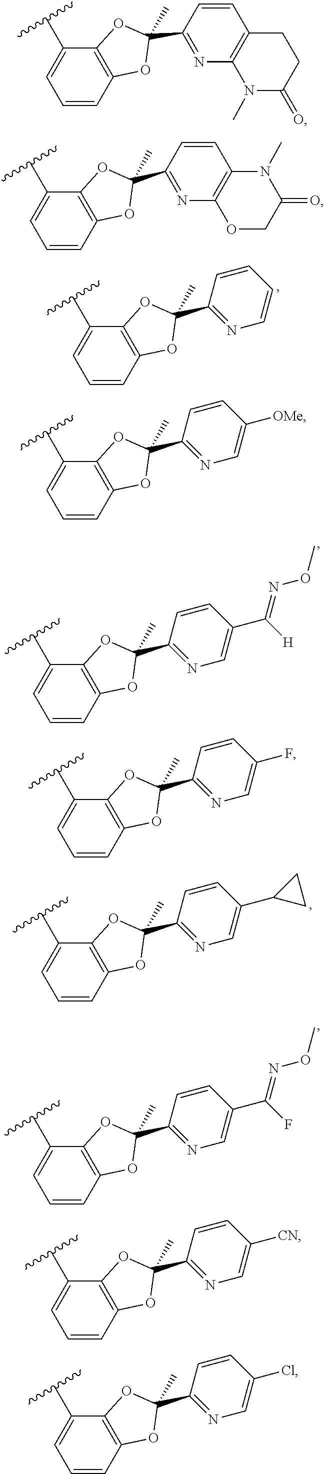

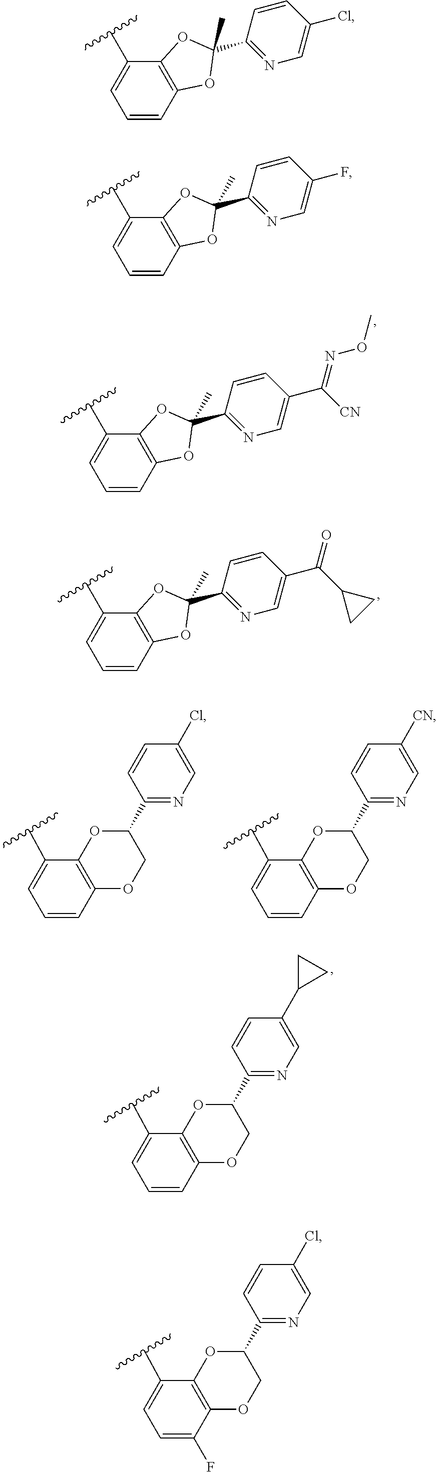

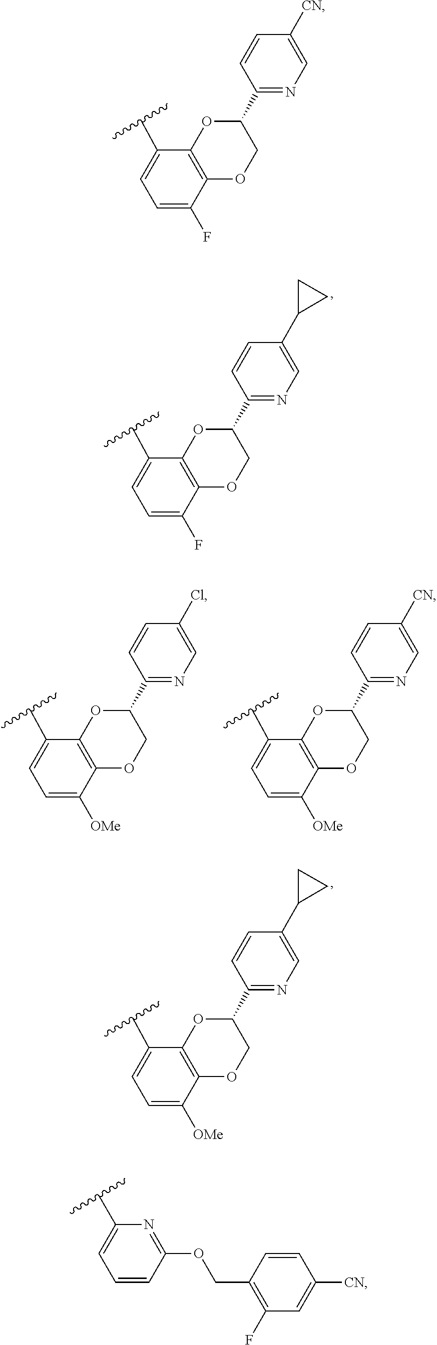 [SMILES: *c1ccc(F)c2c1O[C@H](c1ccc(C#N)cn1)CO2 ; *c1ccc(F)c2c1O[C@H](c1ccc(C3CC3)cn1)CO2 ; *c1ccc(OC)c2c1O[C@H](c1ccc(Cl)cn1)CO2 ; *c1ccc(OC)c2c1O[C@H](c1ccc(C#N)cn1)CO2 ; *c1ccc(OC)c2c1O[C@H](c1ccc(C3CC3)cn1)CO2 ; *c1cccc(OCc2ccc(C#N)cc2F)n1]

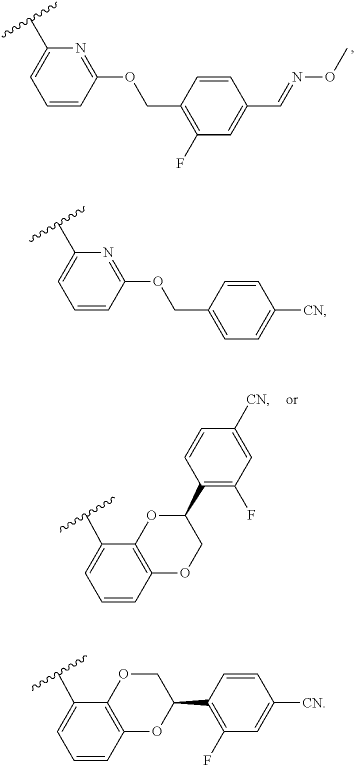 [SMILES: *c1cccc(OCc2ccc(/C=N/OC)cc2F)n1 ; *c1cccc(OCc2ccc(C#N)cc2)n1 ; *c1cccc2c1O[C@@H](c1ccc(C#N)cc1F)CO2 ; *c1cccc2c1OC[C@@H](c1ccc(C#N)cc1F)O2]

The present disclosure contemplates the combination of any one of Formula (I**), (II*), (I*), (I″), (I′″), (T), (I), (IE), (IK), (IL), (IM), (IN), (IO), (IP) (IQ), (IR), (IS), (IT), (IU), (IX), (IY), (IZ), (IAA), (LAB), (IAC), (LAD), (IAE), or (LAI) with any one of the following

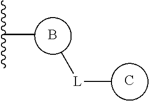

moieties:

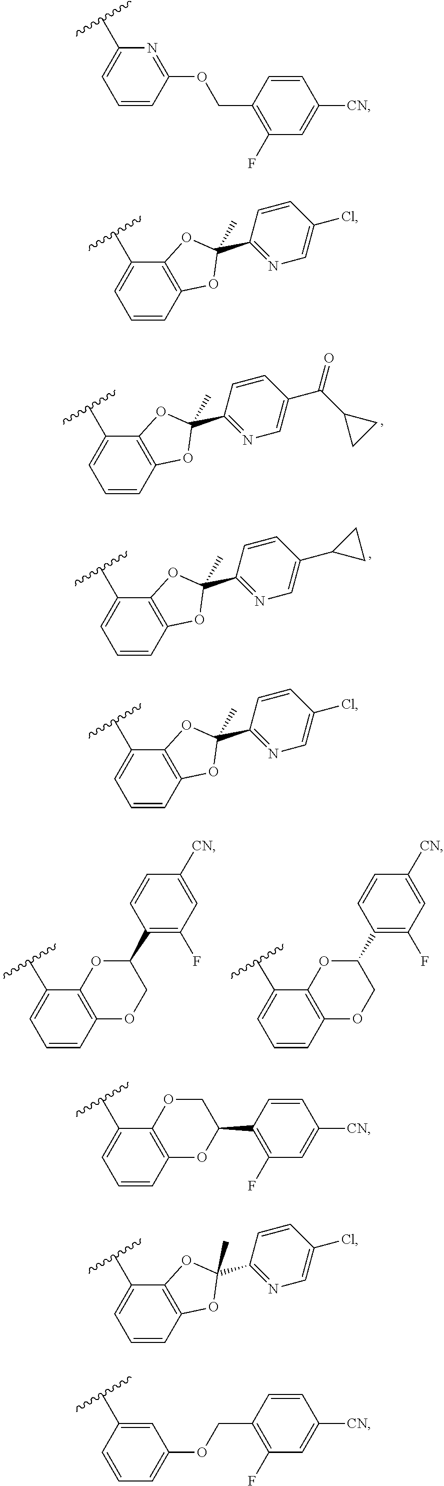

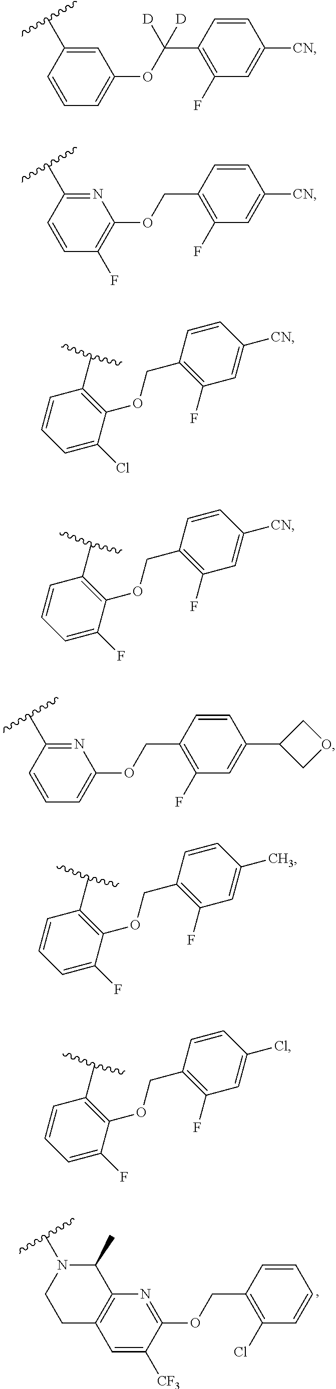

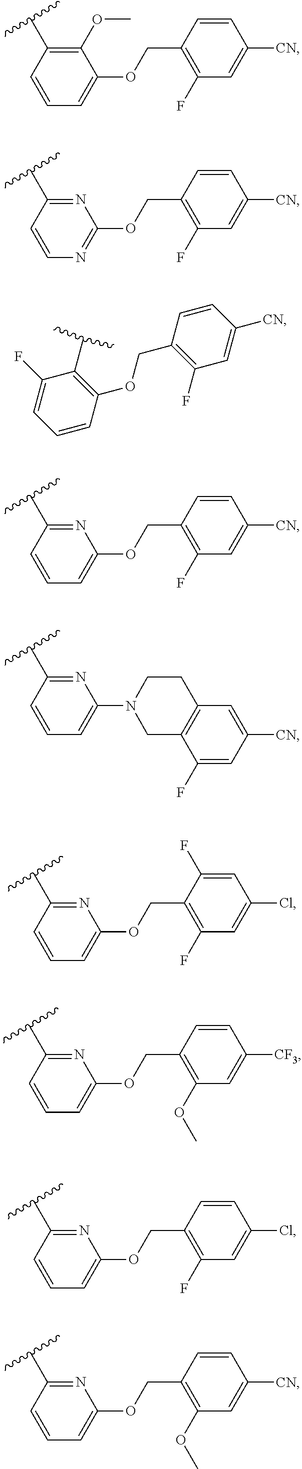

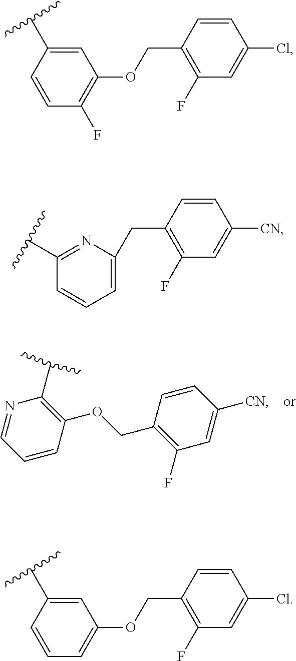 [SMILES: *c1ccc(F)c(OCc2ccc(Cl)cc2F)c1 ; *c1cccc(Cc2ccc(C#N)cc2F)n1 ; *c1ncccc1OCc1ccc(C#N)cc1F ; *c1cccc(OCc2ccc(Cl)cc2F)c1]

It is understood that, for a compound of the present disclosure, variables X¹, X², X³, X⁴, X³, X⁶, R¹, R², R³, R⁴, R⁵, R⁶, R⁷, R⁸, R⁹, R¹⁰, R¹¹, R¹², L, L′, n, Ring A, Ring B, and Ring C can each be, where applicable, selected from the groups described herein, and any group described herein for any of variables X¹, X², X³, X⁴, X³, X⁶, R¹, R², R³, R⁴, R⁵, R⁶, R⁷, R⁸, R⁹, R¹⁰, R¹¹, R¹², L, L′, n, Ring A, Ring B, and Ring C can be combined, where applicable, with any group described herein for one or more of the remainder of variables X¹, X², X³, X⁴, X⁵, X⁶, R¹, R², R³, R⁴, R⁵, R⁶, R⁷, R⁸, R⁹, R¹⁰, R¹¹, R¹², L, L′, n, Ring A, Ring B, and Ring C.

In some embodiments, the compound is selected from the compounds described in Table 1 and prodrugs and pharmaceutically acceptable salts thereof.

In some embodiments, the compound is selected from the compounds described in Table 1 and pharmaceutically acceptable salts thereof.

In some embodiments, the compound is selected from the prodrugs of compounds described in Table 1 and pharmaceutically acceptable salts thereof.

In some embodiments, the compound is selected from the compounds described in Table 1.

TABLE 1

| Compound | | |
|---|---|---|
| No. | Name | Structure |
| 1 | 2-((4-(6-((4-cyano-2- fluorobenzyl)oxy)pyridin-2- yl)piperidin-1-yl)methyl)-4- methoxy-1-(thiazol-5- ylmethyl)-1H- benzo[d]imidazole-6- carboxylic acid | 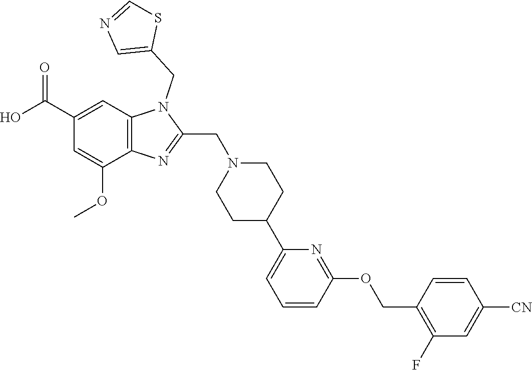 [SMILES: COc1cc(C(=O)O)cc2c1nc(CN1CCC(c3cccc(OCc4ccc(C#N)cc4F)n3)CC1)n2Cc1cncs1] |
| 2 | (S)-2-((4-(2-(5- chloropyridin-2-yl)-2- methylbenzo[d][1,3]dioxol- 4-yl)piperidin-1-yl)methyl)- 4-(2-methoxyethoxy)-1- (thiazol-5-ylmethyl)-1H- benzo[d]imidazole-6- carboxylic acid | 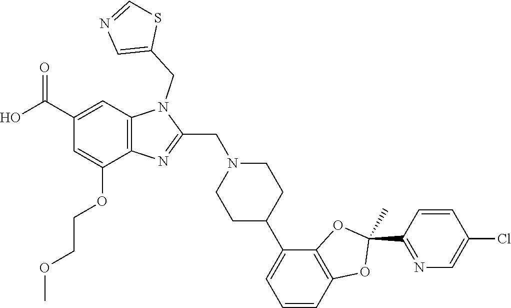 [SMILES: COCCOc1cc(C(=O)O)cc2c1nc(CN1CCC(c3cccc4c3O[C@@](C)(c3ccc(Cl)cn3)O4)CC1)n2Cc1cncs1] |
| 3 | (R)-2-((4-(2-(5- chloropyridin-2-yl)-2- methylbenzo[d][1,3]dioxol- 4-yl)piperidin-1-yl)methyl)- 4-(2-methoxyethoxy)-1- (thiazol-5-ylmethyl)-1H- benzo[d]imidazole-6- carboxylic acid | 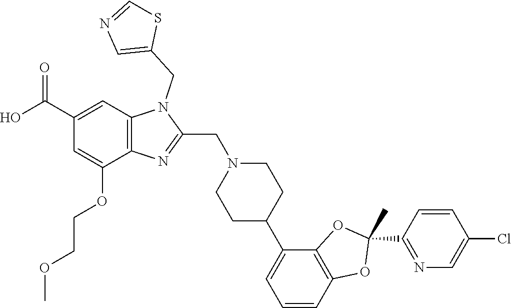 [SMILES: COCCOc1cc(C(=O)O)cc2c1nc(CN1CCC(c3cccc4c3O[C@](C)(c3ccc(Cl)cn3)O4)CC1)n2Cc1cncs1] |
| 4 | (S)-4-((2,5,8,11,14- pentaoxahexadecan-16- yl)oxy)-2-((4-(2-(5- chloropyridin-2-yl)-2- methylbenzo[d][1,3]dioxol- 4-yl)piperidin-1-yl)methyl)- 1-(thiazol-5-ylmethyl)-1H- benzo[d]imidazole-6- carboxylic acid | 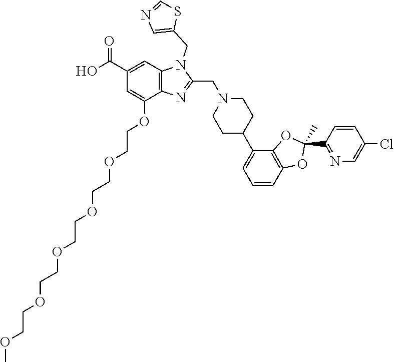 [SMILES: COCCOCCOCCOCCOCCOc1cc(C(=O)O)cc2c1nc(CN1CCC(c3cccc4c3O[C@@](C)(c3ccc(Cl)cn3)O4)CC1)n2Cc1cncs1] |
| 5 | (R)-4-((2,5,8,11,14- pentaoxahexadecan-16- yl)oxy)-2-((4-(2-(5- chloropyridin-2-yl)-2- methylbenzo[d][1,3]dioxol- 4-yl)piperidin-1-yl)methyl)- 1-(thiazol-5-ylmethyl)-1H- benzo[d]imidazole-6- carboxylic acid | 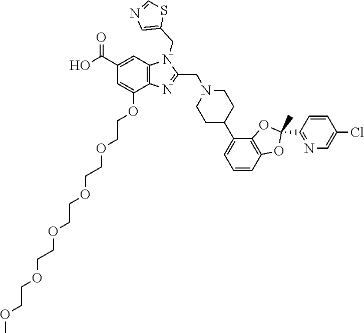 [SMILES: COCCOCCOCCOCCOCCOc1cc(C(=O)O)cc2c1nc(CN1CCC(c3cccc4c3O[C@](C)(c3ccc(Cl)cn3)O4)CC1)n2Cc1cncs1] |
| 6 | (S)-2-((4-(2-(5- chloropyridin-2-yl)-2- methylbenzo[d][1,3]dioxol- 4-yl)piperidin-1-yl)methyl)- 4-cyclopropoxy-1-(thiazol-5- ylmethyl)-1H- benzo[d]imidazole-6- carboxylic acid | 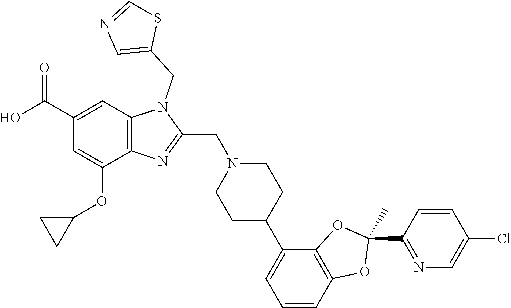 [SMILES: C[C@]1(c2ccc(Cl)cn2)Oc2cccc(C3CCN(Cc4nc5c(OC6CC6)cc(C(=O)O)cc5n4Cc4cncs4)CC3)c2O1] |
| 7 | (R)-2-((4-(2-(5- chloropyridin-2-yl)-2- methylbenzo[d][1,3]dioxol- 4-yl)piperidin-1-yl)methyl)- 4-cyclopropoxy-1-(thiazol-5- ylmethyl)-1H- benzo[d]imidazole-6- carboxylic acid | 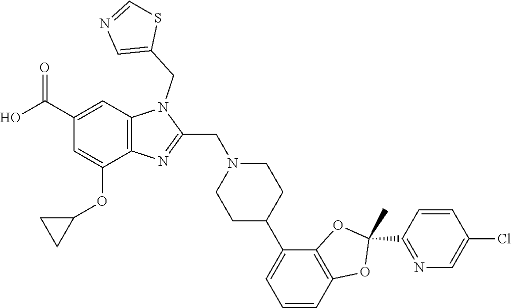 [SMILES: C[C@@]1(c2ccc(Cl)cn2)Oc2cccc(C3CCN(Cc4nc5c(OC6CC6)cc(C(=O)O)cc5n4Cc4cncs4)CC3)c2O1] |
| 8 | (S)-2-((4-(6-((4-cyano-2- fluorobenzyl)oxy)pyridin-2- yl)piperidin-1-yl)methyl)-4- methoxy-1-(oxetan-2- ylmethyl)-1H- benzo[d]imidazole-6- carboxylic acid | 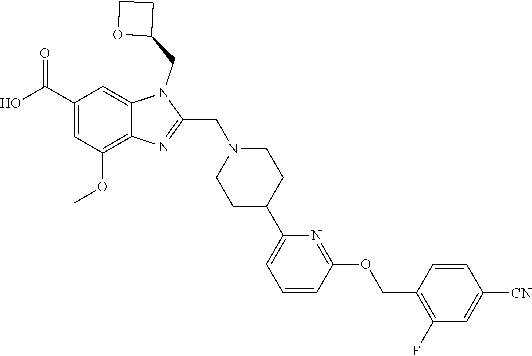 [SMILES: COc1cc(C(=O)O)cc2c1nc(CN1CCC(c3cccc(OCc4ccc(C#N)cc4F)n3)CC1)n2C[C@@H]1CCO1] |
| 9 | (S,E)-2-((4-(6-((2-fluoro-4- ((methoxyimino)methyl) benzyl)oxy)pyridin-2- yl)piperidin-1-yl)methyl)-4- methoxy-1-(oxetan-2- ylmethyl)-1H- benzo[d]imidazole-6- carboxylic acid | 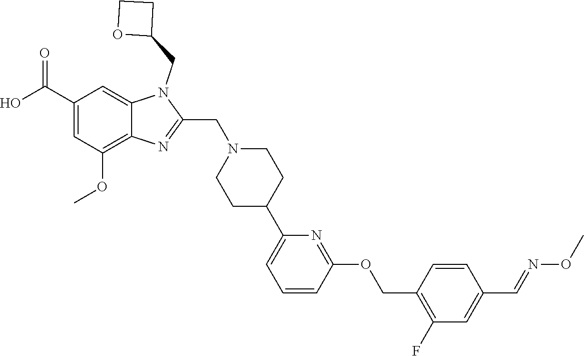 [SMILES: CO/N=C/c1ccc(COc2cccc(C3CCN(Cc4nc5c(OC)cc(C(=O)O)cc5n4C[C@@H]4CCO4)CC3)n2)c(F)c1] |
| 10 | 2-((4-(6-((4-Cyano-2- fluorobenzyl)oxy)pyridin-2- yl)piperidin-1-yl)methyl)-4- ethoxy-1-(thiazol-5- ylmethyl)-1H- benzo[d]imidazole-6- carboxylic acid | 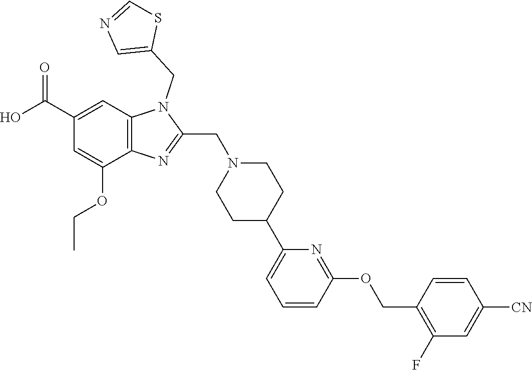 [SMILES: CCOc1cc(C(=O)O)cc2c1nc(CN1CCC(c3cccc(OCc4ccc(C#N)cc4F)n3)CC1)n2Cc1cncs1] |
| 11 | 2-((4-(6-((4-Cyano-2- fluorobenzyl)oxy)pyridin-2- yl)piperidin-1-yl)methyl)-4- (2-methoxyethoxy)-1- (thiazol-5-ylmethyl)-1H- benzo[d]imidazole-6- carboxylic acid | 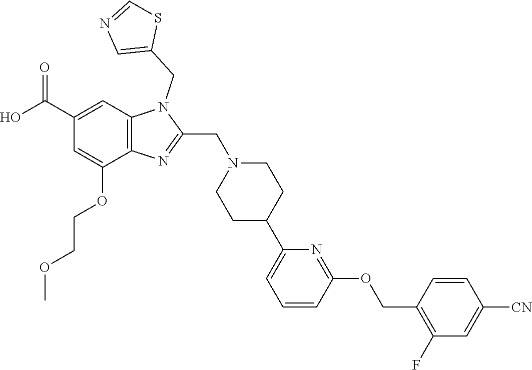 [SMILES: COCCOc1cc(C(=O)O)cc2c1nc(CN1CCC(c3cccc(OCc4ccc(C#N)cc4F)n3)CC1)n2Cc1cncs1] |
| 12 | (S)-2-((4-(6-((4- Cyanobenzyl)oxy)pyridin-2- yl)piperidin-1-yl)methyl)-4- (2-(2- methoxyethoxy)ethoxy)-1- (oxetan-2-ylmethyl)-1H- benzo[d]imidazole-6- carboxylic acid | 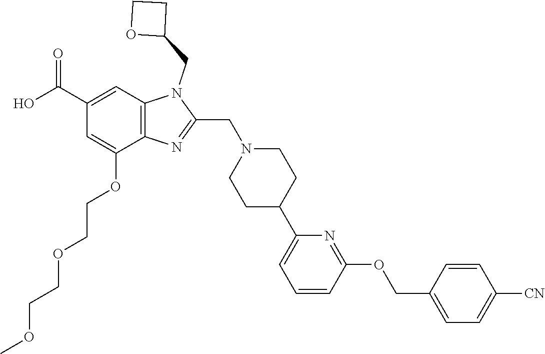 [SMILES: COCCOCCOc1cc(C(=O)O)cc2c1nc(CN1CCC(c3cccc(OCc4ccc(C#N)cc4)n3)CC1)n2C[C@@H]1CCO1] |
| 13 | (S)-2-((4-(6-((4- Cyanobenzyl)oxy)pyridin-2- yl)piperidin-1-yl)methyl)-4- (2-(2-(2- methoxyethoxy)ethoxy)ethox y)-1-(oxetan-2-ylmethyl)- 1H-benzo[d]imidazole-6- carboxylic acid | 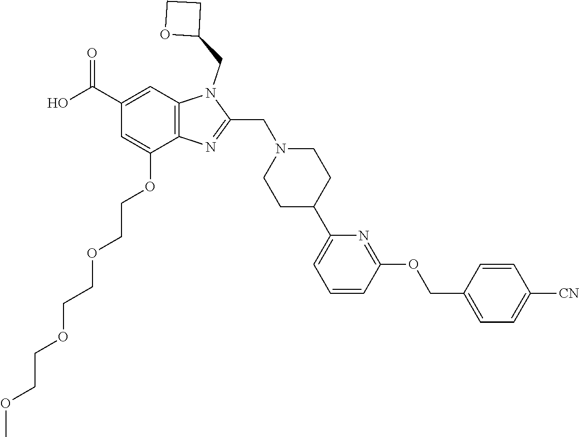 [SMILES: COCCOCCOCCOc1cc(C(=O)O)cc2c1nc(CN1CCC(c3cccc(OCc4ccc(C#N)cc4)n3)CC1)n2C[C@@H]1CCO1] |
| 14 | (S)-4-((2,5,8,11- Tetraoxatridecan-13-yl)oxy)- 2-((4-(6-((4- cyanobenzyl)oxy)pyridin-2- yl)piperidin-1-yl)methyl)-1- (oxetan-2-ylmethyl)-1H- benzo[d]imidazole-6- carboxylic acid | 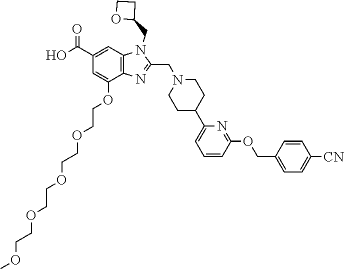 [SMILES: COCCOCCOCCOCCOc1cc(C(=O)O)cc2c1nc(CN1CCC(c3cccc(OCc4ccc(C#N)cc4)n3)CC1)n2C[C@@H]1CCO1] |
| 15 | (S)-4-((2,5,8,11,14- Pentaoxahexadecan-16- yl)oxy)-2-((4-(6-((4- cyanobenzyl)oxy)pyridin-2- yl)piperidin-1-yl)methyl)-1- (oxetan-2-ylmethyl)-1H- benzo[d]imidazole-6- carboxylic acid | 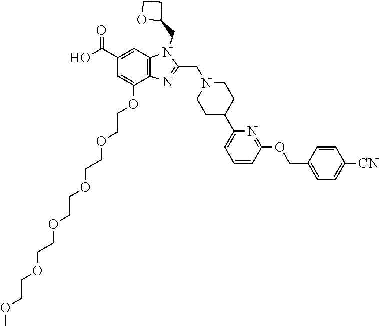 [SMILES: COCCOCCOCCOCCOCCOc1cc(C(=O)O)cc2c1nc(CN1CCC(c3cccc(OCc4ccc(C#N)cc4)n3)CC1)n2C[C@@H]1CCO1] |
| 16 | (S)-4-((2,5,8,11,14,17- Hexaoxanonadecan-19- yl)oxy)-2-((4-(6-((4- cyanobenzyl)oxy)pyridin-2- yl)piperidin-1-yl)methyl)-1- (oxetan-2-ylmethyl)-1H- benzo[d]imidazole-6- carboxylic acid | 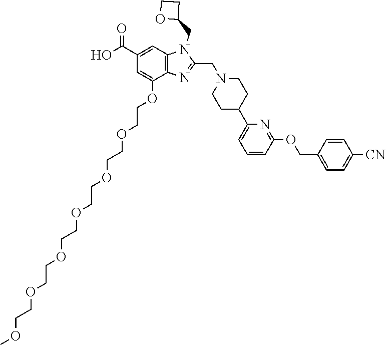 [SMILES: COCCOCCOCCOCCOCCOCCOc1cc(C(=O)O)cc2c1nc(CN1CCC(c3cccc(OCc4ccc(C#N)cc4)n3)CC1)n2C[C@@H]1CCO1] |
| 17 | (S)-4-((2,5,8,11,14,17,20- Heptaoxadocosan-22- yl)oxy)-2-((4-(6-((4- cyanobenzyl)oxy)pyridin-2- yl)piperidin-1-yl)methyl)-1- (oxetan-2-ylmethyl)-1H- benzo[d]imidazole-6- carboxylic acid | 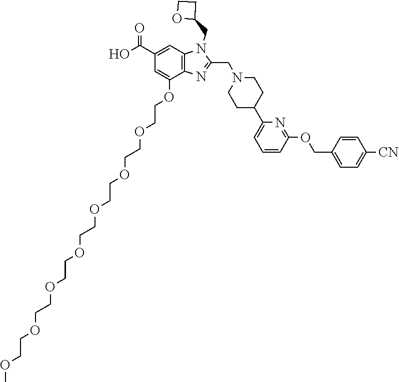 [SMILES: COCCOCCOCCOCCOCCOCCOCCOc1cc(C(=O)O)cc2c1nc(CN1CCC(c3cccc(OCc4ccc(C#N)cc4)n3)CC1)n2C[C@@H]1CCO1] |
| 18 | 2-((4-(6-((4- Cyanobenzyl)oxy)pyridin-2- yl)piperidin-1-yl)methyl)-4- (2,3-dimethoxypropoxy)-1- (((S)-oxetan-2-yl)methyl)- 1H-benzo[d]imidazole-6- carboxylic acid | 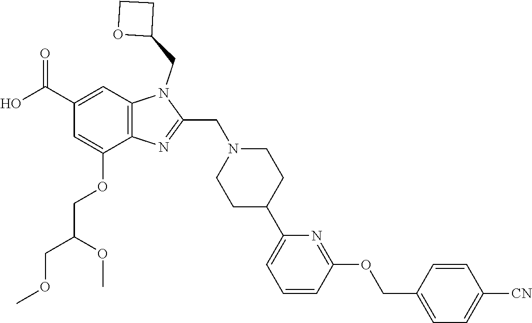 [SMILES: COCC(COc1cc(C(=O)O)cc2c1nc(CN1CCC(c3cccc(OCc4ccc(C#N)cc4)n3)CC1)n2C[C@@H]1CCO1)OC] |
| 19 | 2-((4-(6-((4- cyanobenzyl)oxy)pyridin-2- yl)piperidin-1-yl)methyl)-4- (3-(2,3-dimethoxypropoxy)- 2-methoxypropoxy)-1-(((S)- oxetan-2-yl)methyl)-1H- benzo[d]imidazole-6- carboxylic acid | 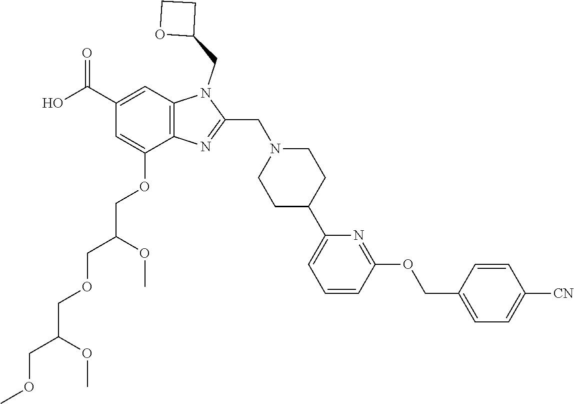 [SMILES: COCC(COCC(COc1cc(C(=O)O)cc2c1nc(CN1CCC(c3cccc(OCc4ccc(C#N)cc4)n3)CC1)n2C[C@@H]1CCO1)OC)OC] |
| 20 | 2-((4-(6-((4-Cyano-2- fluorobenzyl)oxy)pyridin-2- yl)piperidin-1-yl)methyl)-7- methoxy-3-(thiazol-5- ylmethyl)-3H-imidazo[4,5- blpyridine-5-carboxylic acid | 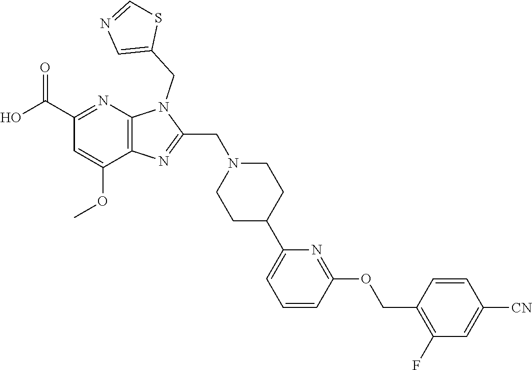 [SMILES: COc1cc(C(=O)O)nc2c1nc(CN1CCC(c3cccc(OCc4ccc(C#N)cc4F)n3)CC1)n2Cc1cncs1] |
| 21 | 2-((4-(6-((4-Cyano-2- fluorobenzyl)oxy)pyridin-2- yl)piperidin-1-yl)methyl)-7- ethoxy-3-(thiazol-5- ylmethyl)-3H-imidazo[4,5- b]pyridine-5-carboxylic acid | 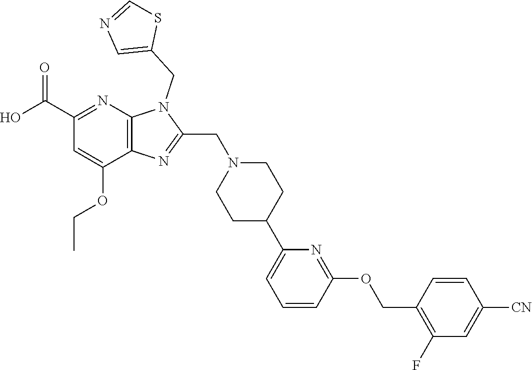 [SMILES: CCOc1cc(C(=O)O)nc2c1nc(CN1CCC(c3cccc(OCc4ccc(C#N)cc4F)n3)CC1)n2Cc1cncs1] |
| 22 | 2-((4-(6-((4-Cyano-2- fluorobenzyl)oxy)pyridin-2- yl)piperidin-1-yl)methyl)-7- (2-methoxyethoxy)-3- (thiazol-5-ylmethyl)-3H- imidazo[4,5-b]pyridine-5- carboxylic acid | 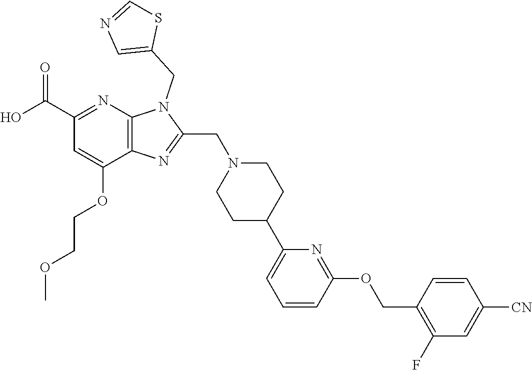 [SMILES: COCCOc1cc(C(=O)O)nc2c1nc(CN1CCC(c3cccc(OCc4ccc(C#N)cc4F)n3)CC1)n2Cc1cncs1] |
| 23 | 2-((4-((S)-2-(5- Chloropyridin-2-yl)-2- methylbenzo[d][1,3]dioxol- 4-yl)piperidin-1-yl)methyl)- 4-methoxy-1-(((S)-oxetan-2- yl)methyl)-1H- benzo[d]imidazole-6- carboxylic acid | 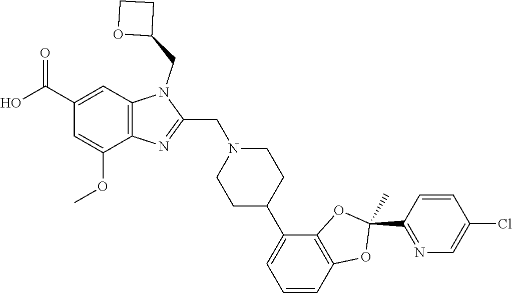 [SMILES: COc1cc(C(=O)O)cc2c1nc(CN1CCC(c3cccc4c3O[C@@](C)(c3ccc(Cl)cn3)O4)CC1)n2C[C@@H]1CCO1] |
| 24 | 2-((4-((S)-2-(5- Chloropyridin-2-yl)-2- methylbenzo[d][1,3]dioxol- 4-yl)piperidin-1-yl)methyl)- 4-(2-methoxyethoxy)-1- (((S)-oxetan-2-yl)methyl)- 1H-benzo[d]imidazole-6- carboxylic acid | 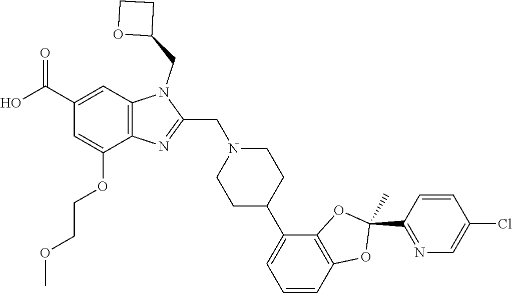 [SMILES: COCCOc1cc(C(=O)O)cc2c1nc(CN1CCC(c3cccc4c3O[C@@](C)(c3ccc(Cl)cn3)O4)CC1)n2C[C@@H]1CCO1] |
| 25 | 2-((4-((S)-2-(5- (Cyclopropanecarbonyl) pyridin-2-yl)-2- methylbenzo[d][1,3]dioxol- 4-yl)piperidin-1-yl)methyl)- 4-methoxy-1-(((S)-oxetan-2- yl)methyl)-1H- benzo[d]imidazole-6- carboxylic acid | 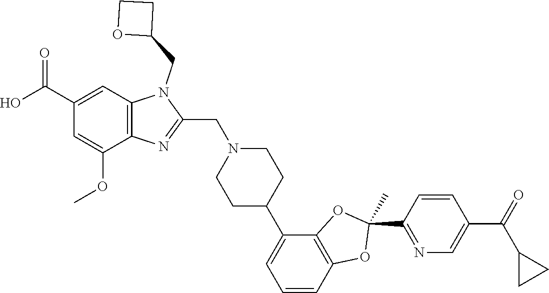 [SMILES: COc1cc(C(=O)O)cc2c1nc(CN1CCC(c3cccc4c3O[C@@](C)(c3ccc(C(=O)C5CC5)cn3)O4)CC1)n2C[C@@H]1CCO1] |
| 26 | 2-((4-((S)-2-(5- Cyclopropylpyridin-2-yl)-2- methylbenzo[d][1,3]dioxol- 4-yl)piperidin-1-yl)methyl)- 4-methoxy-1-(((S)-oxetan-2- yl)methyl)-1H- benzo[d]imidazole-6- carboxylic acid | 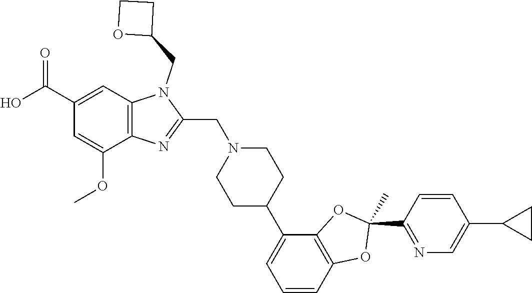 [SMILES: COc1cc(C(=O)O)cc2c1nc(CN1CCC(c3cccc4c3O[C@@](C)(c3ccc(C5CC5)cn3)O4)CC1)n2C[C@@H]1CCO1] |
| 27 | (S)-2-((4-(2-(5- Chloropyridin-2-yl)-2- methylbenzo[d][1,3]dioxol- 4-yl)piperidin-1-yl)methyl)- 4-methoxy-1-(2- methoxyethyl)-1H- benzo[d]imidazole-6- carboxylic acid | 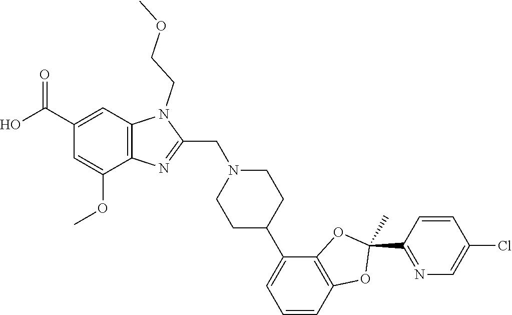 [SMILES: COCCn1c(CN2CCC(c3cccc4c3O[C@@](C)(c3ccc(Cl)cn3)O4)CC2)nc2c(OC)cc(C(=O)O)cc21] |
| 28 | 2-((4-((S)-2-(5- Chloropyridin-2-yl)-2- methylbenzo[d][1,3]dioxol- 4-yl)piperidin-1-yl)methyl)- 1-(((S)-1,1-dioxidothietan-2- yl)methyl)-4-methoxy-1H- benzo[d]imidazole-6- carboxylic acid | 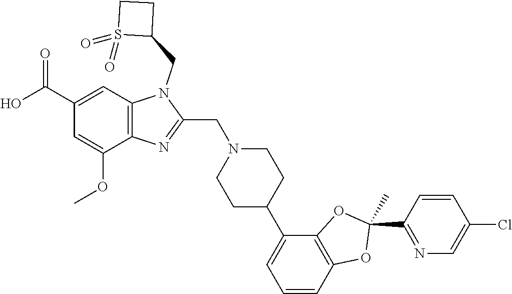 [SMILES: COc1cc(C(=O)O)cc2c1nc(CN1CCC(c3cccc4c3O[C@@](C)(c3ccc(Cl)cn3)O4)CC1)n2C[C@@H]1CCS1(=O)=O] |
| 29 | 2-((4-((S)-2-(5- Chloropyridin-2-yl)-2- methylbenzo[d][1,3]dioxol- 4-yl)piperidin-1-yl)methyl)- 1-(((2S)-1-imino-1-oxido- 116-thietan-2-yl)methyl)-4- methoxy-1H- benzo[d]imidazole-6- carboxylic acid | 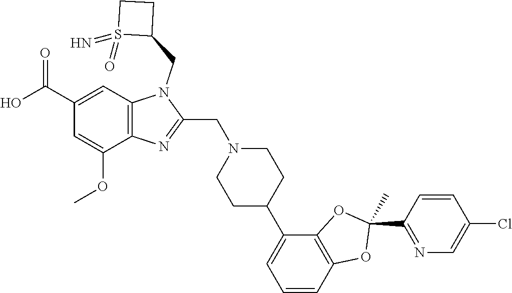 [SMILES: COc1cc(C(=O)O)cc2c1nc(CN1CCC(c3cccc4c3O[C@@](C)(c3ccc(Cl)cn3)O4)CC1)n2C[C@@H]1CCS1(=N)=O] |
| 30 | (S)-2-((4-(2-(5- Chloropyridin-2-yl)-2- methylbenzo[d][1,3]dioxol- 4-yl)piperidin-1-yl)methyl)- 4-(2-methoxyethoxy)-1- methyl-1H- benzo[d]imidazole-6- carboxylic acid | 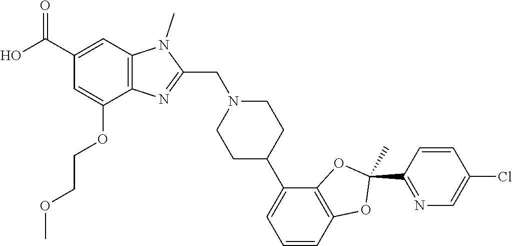 [SMILES: COCCOc1cc(C(=O)O)cc2c1nc(CN1CCC(c3cccc4c3O[C@@](C)(c3ccc(Cl)cn3)O4)CC1)n2C] |
| 31 | (S)-2-((4-(2-(5- Chloropyridin-2-yl)-2- methylbenzo[d][1,3]dioxol- 4-yl)piperidin-1-yl)methyl)- 7-(2-methoxyethoxy)-3- methyl-3H-imidazo[4,5- b]pyridine-5-carboxylic acid | 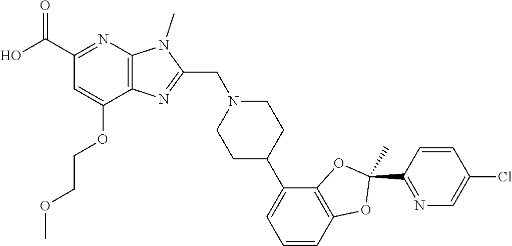 [SMILES: COCCOc1cc(C(=O)O)nc2c1nc(CN1CCC(c3cccc4c3O[C@@](C)(c3ccc(Cl)cn3)O4)CC1)n2C] |
| 32 | (S)-2-((4-(2-(5- Chloropyridin-2-yl)-2- methylbenzo[d][1,3]dioxol- 4-yl)piperidin-1-yl)methyl)- 4-methoxy-1-(thiazol-5- ylmethyl)-1H- benzo[d]imidazole-6- carboxylic acid | 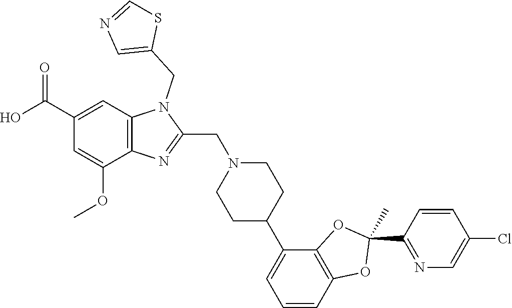 [SMILES: COc1cc(C(=O)O)cc2c1nc(CN1CCC(c3cccc4c3O[C@@](C)(c3ccc(Cl)cn3)O4)CC1)n2Cc1cncs1] |
| 33 | (S)-2-((4-(2-(5- Chloropyridin-2-yl)-2- methylbenzo[d][1,3]dioxol- 4-yl)piperidin-1-yl)methyl)- 4-ethoxy-1-(thiazol-5- ylmethyl)-1H- benzo[d]imidazole-6- carboxylic acid | 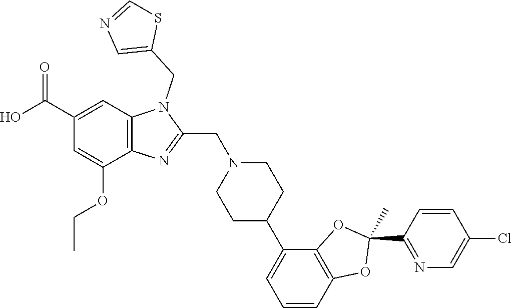 [SMILES: CCOc1cc(C(=O)O)cc2c1nc(CN1CCC(c3cccc4c3O[C@@](C)(c3ccc(Cl)cn3)O4)CC1)n2Cc1cncs1] |
| 34 | (S)-2-((4-(2-(5- Chloropyridin-2-yl)-2- methylbenzo[d][1,3]dioxol- 4-yl)piperidin-1-yl)methyl)- 4-isopropoxy-1-(thiazol-5- ylmethyl)-1H- benzo[d]imidazole-6- carboxylic acid | 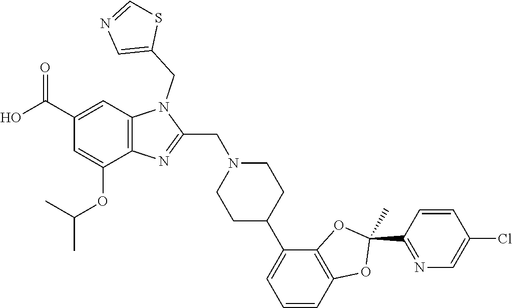 [SMILES: CC(C)Oc1cc(C(=O)O)cc2c1nc(CN1CCC(c3cccc4c3O[C@@](C)(c3ccc(Cl)cn3)O4)CC1)n2Cc1cncs1] |
| 35 | (S)-2-((4-(2-(5- Chloropyridin-2-yl)-2- methylbenzo[d][1,3]dioxol- 4-yl)piperidin-1-yl)methyl)- 4-(cyclopropylmethoxy)-1- (thiazol-5-ylmethyl)-1H- benzo[d]imidazole-6- carboxylic acid | 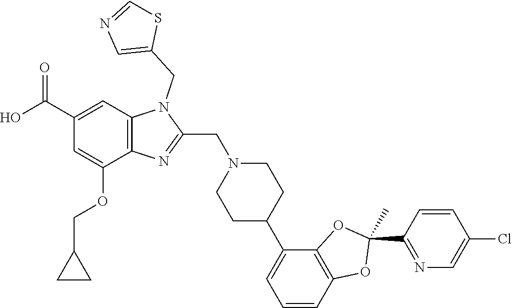 [SMILES: C[C@]1(c2ccc(Cl)cn2)Oc2cccc(C3CCN(Cc4nc5c(OCC6CC6)cc(C(=O)O)cc5n4Cc4cncs4)CC3)c2O1] |
| 36 | (S)-2-((4-(2-(5- Chloropyridin-2-yl)-2- methylbenzo[d][1,3]dioxol- 4-yl)piperidin-1-yl)methyl)- 4-(oxetan-3-yloxy)-1- (thiazol-5-ylmethyl)-1H- benzo[d]imidazole-6- carboxylic acid | 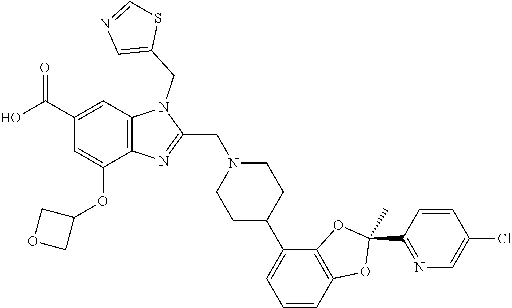 [SMILES: C[C@]1(c2ccc(Cl)cn2)Oc2cccc(C3CCN(Cc4nc5c(OC6COC6)cc(C(=O)O)cc5n4Cc4cncs4)CC3)c2O1] |
| 37 | (S)-2-((4-(2-(5- Chloropyridin-2-yl)-2- methylbenzo[d][1,3]dioxol- 4-yl)piperidin-1-yl)methyl)- 4-(oxetan-3-ylmethoxy)-1- (thiazol-5-ylmethyl)-1H- benzo[d]imidazole-6- carboxylic acid | 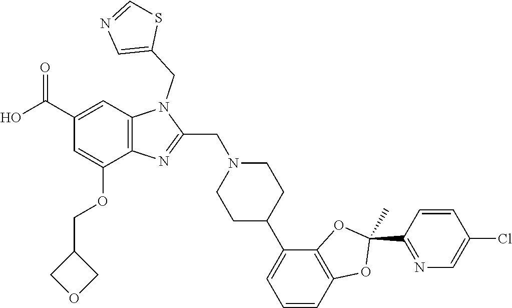 [SMILES: C[C@]1(c2ccc(Cl)cn2)Oc2cccc(C3CCN(Cc4nc5c(OCC6COC6)cc(C(=O)O)cc5n4Cc4cncs4)CC3)c2O1] |
| 38 | 2-((4-((S)-2-(5- Chloropyridin-2-yl)-2- methylbenzo[d][1,3]dioxol- 4-yl)piperidin-1-yl)methyl)- 4-(oxetan-2-ylmethoxy)-1- (thiazol-5-ylmethyl)-1H- benzo[d]imidazole-6- carboxylic acid | 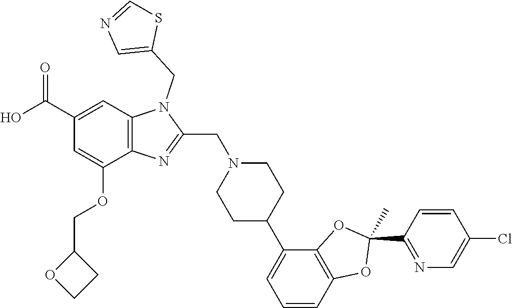 [SMILES: C[C@]1(c2ccc(Cl)cn2)Oc2cccc(C3CCN(Cc4nc5c(OCC6CCO6)cc(C(=O)O)cc5n4Cc4cncs4)CC3)c2O1] |
| 39 | (S)-2-((4-(2-(5- Chloropyridin-2-yl)-2- methylbenzo[d][1,3]dioxol- 4-yl)piperidin-1-yl)methyl)- 4-((1- cyanocyclopropyl)methoxy)- 1-(thiazol-5-ylmethyl)-1H- benzo[d]imidazole-6- carboxylic acid | 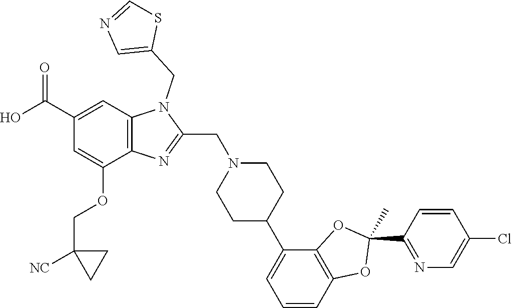 [SMILES: C[C@]1(c2ccc(Cl)cn2)Oc2cccc(C3CCN(Cc4nc5c(OCC6(C#N)CC6)cc(C(=O)O)cc5n4Cc4cncs4)CC3)c2O1] |
| 40 | 2-((4-((S)-2-(5- Chloropyridin-2-yl)-2- methylbenzo[d][1,3]dioxol- 4-yl)piperidin-1-yl)methyl)- 7-methoxy-3-(((S)-oxetan-2- yl)methyl)-3H-imidazo[4,5- b]pyridine-5-carboxylic acid | 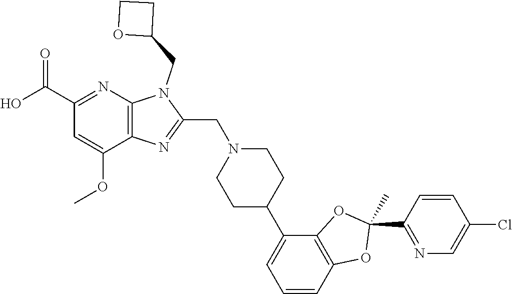 [SMILES: COc1cc(C(=O)O)nc2c1nc(CN1CCC(c3cccc4c3O[C@@](C)(c3ccc(Cl)cn3)O4)CC1)n2C[C@@H]1CCO1] |
| 41 | 2-((4-((S)-2-(5- Chloropyridin-2-yl)-2- methylbenzo[d][1,3]dioxol- 4-yl)piperidin-1-yl)methyl)- 7-(2-methoxyethoxy)-3- (((S)-oxetan-2-yl)methyl)- 3H-imidazo[4,5-b]pyridine- 5-carboxylic acid | 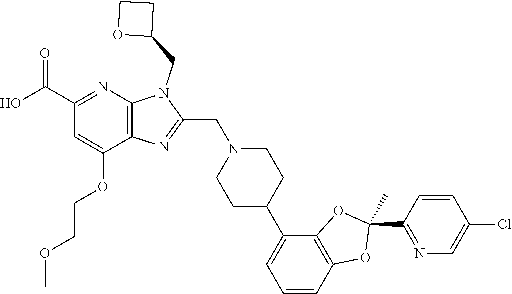 [SMILES: COCCOc1cc(C(=O)O)nc2c1nc(CN1CCC(c3cccc4c3O[C@@](C)(c3ccc(Cl)cn3)O4)CC1)n2C[C@@H]1CCO1] |
| 42 | 2-((4-((S)-2-(5- Chloropyridin-2-yl)-2- methylbenzo[d][1,3]dioxol- 4-yl)piperidin-1-yl)methyl)- 7-(2-fluoroethoxy)-3-(((S)- oxetan-2-yl)methyl)-3H- imidazo[4,5-b]pyridine-5- carboxylic acid | 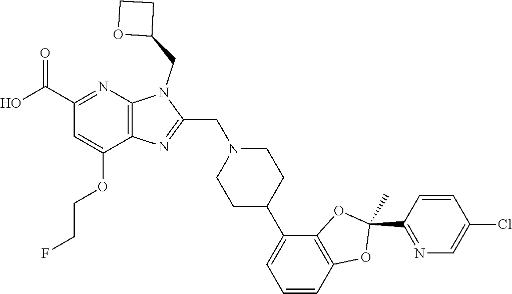 [SMILES: C[C@]1(c2ccc(Cl)cn2)Oc2cccc(C3CCN(Cc4nc5c(OCCF)cc(C(=O)O)nc5n4C[C@@H]4CCO4)CC3)c2O1] |
| 43 | (S)-2-((4-(2-(5- Chloropyridin-2-yl)-2- methylbenzo[d][1,3]dioxol- 4-yl)piperidin-1-yl)methyl)- 7-methoxy-3-(2- methoxyethyl)-3H- imidazo[4,5-b]pyridine-5- carboxylic acid | 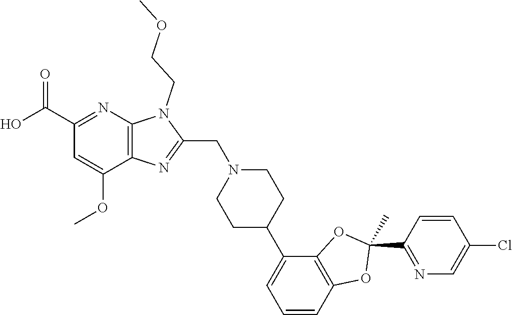 [SMILES: COCCn1c(CN2CCC(c3cccc4c3O[C@@](C)(c3ccc(Cl)cn3)O4)CC2)nc2c(OC)cc(C(=O)O)nc21] |
| 44 | (S)-2-((4-(2-(5- Chloropyridin-2-yl)-2- methylbenzo[d][1,3]dioxol- 4-yl)piperidin-1-yl)methyl)- 7-(2-methoxyethoxy)-3- methyl-3H-imidazo[4,5- b]pyridine-5-carboxylic acid | 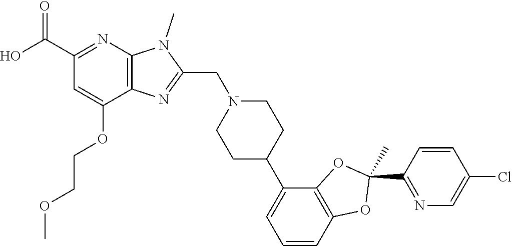 [SMILES: COCCOc1cc(C(=O)O)nc2c1nc(CN1CCC(c3cccc4c3O[C@@](C)(c3ccc(Cl)cn3)O4)CC1)n2C] |
| 45 | (S)-2-((4-(2-(5- Chloropyridin-2-yl)-2- methylbenzo[d][1,3]dioxol- 4-yl)piperidin-1-yl)methyl)- 7-methoxy-3-(thiazol-5- ylmethyl)-3H-imidazo[4,5- b]pyridine-5-carboxylic acid | 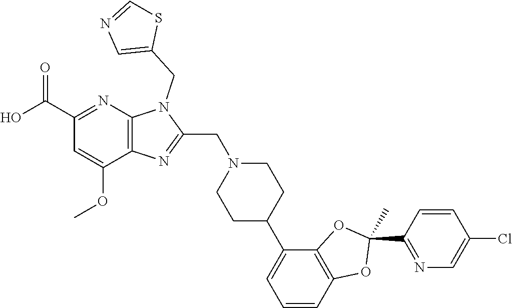 [SMILES: COc1cc(C(=O)O)nc2c1nc(CN1CCC(c3cccc4c3O[C@@](C)(c3ccc(Cl)cn3)O4)CC1)n2Cc1cncs1] |
| 46 | (S)-2-((4-(2-(5- chloropyridin-2-yl)-2- methylbenzo[d][1,3]dioxol- 4-yl)piperidin-1-yl)methyl)- 7-ethoxy-3-(thiazol-5- ylmethyl)-3H-imidazo[4,5- b]pyridine-5-carboxylic acid | 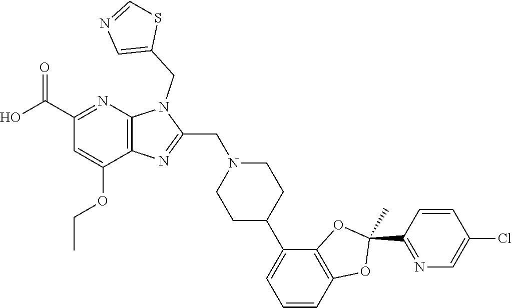 [SMILES: CCOc1cc(C(=O)O)nc2c1nc(CN1CCC(c3cccc4c3O[C@@](C)(c3ccc(Cl)cn3)O4)CC1)n2Cc1cncs1] |
| 47 | (S)-2-((4-(2-(5- Chloropyridin-2-yl)-2- methylbenzo[d][1,3]dioxol- 4-yl)piperidin-1-yl)methyl)- 7-(2-methoxyethoxy)-3- (thiazol-5-ylmethyl)-3H- imidazo[4,5-b]pyridine-5- carboxylic acid | 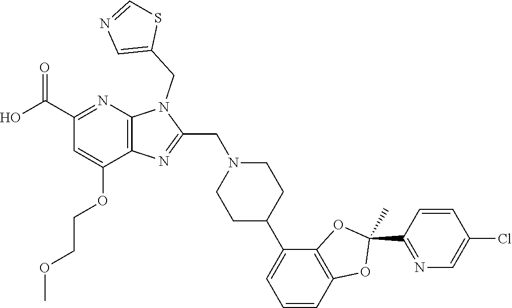 [SMILES: COCCOc1cc(C(=O)O)nc2c1nc(CN1CCC(c3cccc4c3O[C@@](C)(c3ccc(Cl)cn3)O4)CC1)n2Cc1cncs1] |
| 48 | (S)-2-((4-(2-(5- Chloropyridin-2-yl)-2- methylbenzo[d][1,3]dioxol- 4-yl)piperidin-1-yl)methyl)- 7-isopropoxy-3-(thiazol-5- ylmethyl)-3H-imidazo[4,5- b]pyridine-5-carboxylic acid | 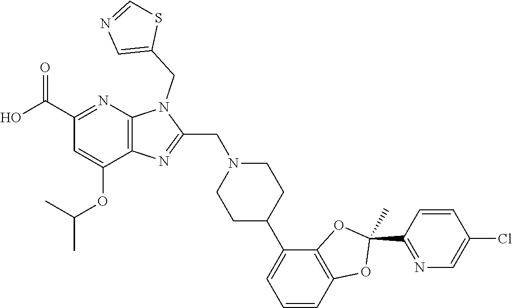 [SMILES: CC(C)Oc1cc(C(=O)O)nc2c1nc(CN1CCC(c3cccc4c3O[C@@](C)(c3ccc(Cl)cn3)O4)CC1)n2Cc1cncs1] |
| 49 | (S)-2-((4-(2-(5- Chloropyridin-2-yl)-2- methylbenzo[d][1,3]dioxol- 4-yl)piperidin-1-yl)methyl)- 7-cyclopropoxy-3-(thiazol-5- ylmethyl)-3H-imidazo[4,5- b]pyridine-5-carboxylic acid | 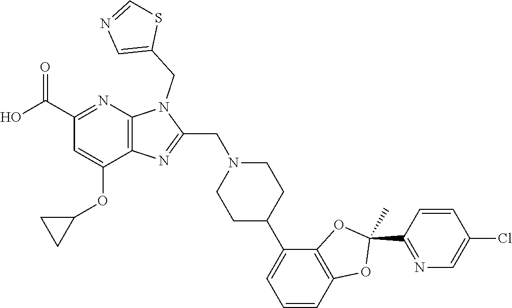 [SMILES: C[C@]1(c2ccc(Cl)cn2)Oc2cccc(C3CCN(Cc4nc5c(OC6CC6)cc(C(=O)O)nc5n4Cc4cncs4)CC3)c2O1] |
| 50 | (S)-2-((4-(2-(5- Chloropyridin-2-yl)-2- methylbenzo[d][1,3]dioxol- 4-yl)piperidin-1-yl)methyl)- 7-(cyclopropylmethoxy)-3- (thiazol-5-ylmethyl)-3H- imidazo[4,5-b]pyridine-5- carboxylic acid | 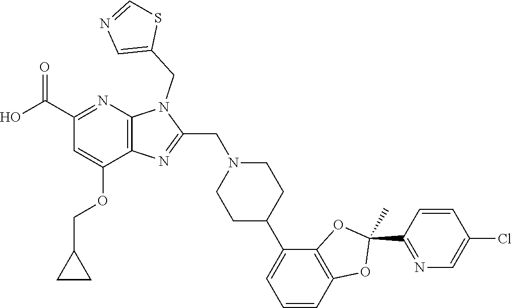 [SMILES: C[C@]1(c2ccc(Cl)cn2)Oc2cccc(C3CCN(Cc4nc5c(OCC6CC6)cc(C(=O)O)nc5n4Cc4cncs4)CC3)c2O1] |
| 51 | (S)-2-((4-(2-(5- Chloropyridin-2-yl)-2- methylbenzo[d][1,3]dioxol- 4-yl)piperidin-1-yl)methyl)- 7-(oxetan-3-yloxy)-3- (thiazol-5-ylmethyl)-3H- imidazo[4,5-b]pyridine-5- carboxylic acid | 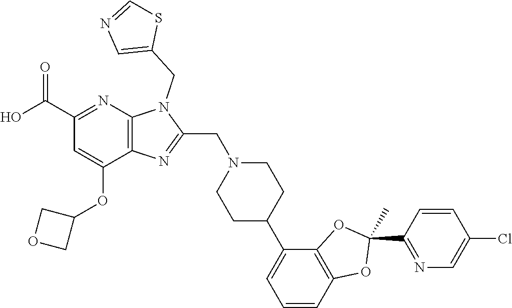 [SMILES: C[C@]1(c2ccc(Cl)cn2)Oc2cccc(C3CCN(Cc4nc5c(OC6COC6)cc(C(=O)O)nc5n4Cc4cncs4)CC3)c2O1] |
| 52 | (S)-2-((4-(2-(5- Chloropyridin-2-yl)-2- methylbenzo[d][1,3]dioxol- 4-yl)piperidin-1-yl)methyl)- 7-(oxetan-3-ylmethoxy)-3- (thiazol-5-ylmethyl)-3H- imidazo[4,5-b]pyridine-5- carboxylic acid | 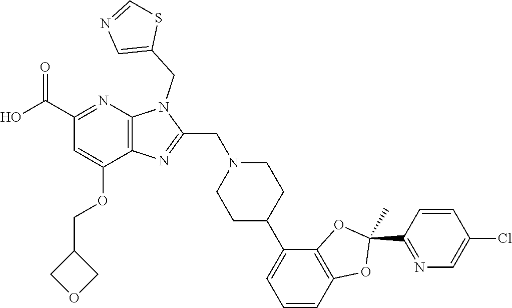 [SMILES: C[C@]1(c2ccc(Cl)cn2)Oc2cccc(C3CCN(Cc4nc5c(OCC6COC6)cc(C(=O)O)nc5n4Cc4cncs4)CC3)c2O1] |
| 53 | 2-((4-((S)-2-(5- Chloropyridin-2-yl)-2- methylbenzo[d][1,3]dioxol- 4-yl)piperidin-1-yl)methyl)- 7-(oxetan-2-ylmethoxy)-3- (thiazol-5-ylmethyl)-3H- imidazo[4,5-b]pyridine-5- carboxylic acid | 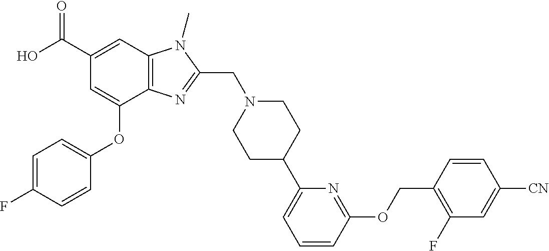 [SMILES: Cn1c(CN2CCC(c3cccc(OCc4ccc(C#N)cc4F)n3)CC2)nc2c(Oc3ccc(F)cc3)cc(C(=O)O)cc21] |
| 54 | (S)-2-((4-(2-(5- Chloropyridin-2-yl)-2- methylbenzo[d][1,3]dioxol- 4-yl)piperidin-1-yl)methyl)- 7-((1- cyanocyclopropyl)methoxy)- 3-(thiazol-5-ylmethyl)-3H- imidazo[4,5-b]pyridine-5- carboxylic acid | 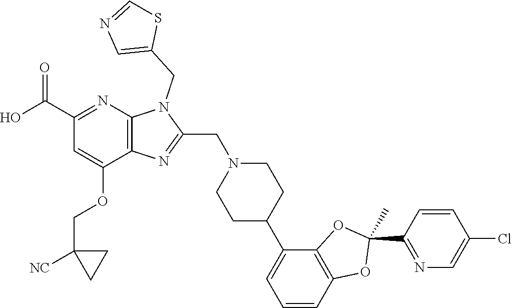 [SMILES: C[C@]1(c2ccc(Cl)cn2)Oc2cccc(C3CCN(Cc4nc5c(OCC6(C#N)CC6)cc(C(=O)O)nc5n4Cc4cncs4)CC3)c2O1] |
| 55 | (S)-2-((4-(6-((4-Cyano-2- fluorobenzyl)oxy)pyridin-2- yl)piperidin-1-yl)methyl)-4- (1,1-difluoroethyl)-1- (oxetan-2-ylmethyl)-1H- benzo[d]imidazole-6- carboxylic acid | 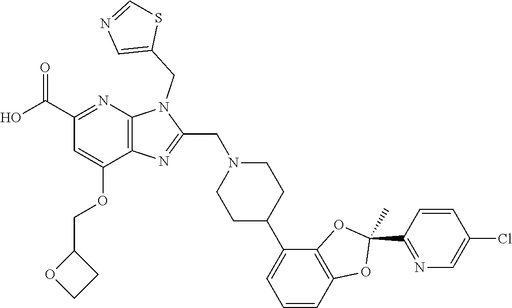 [SMILES: C[C@]1(c2ccc(Cl)cn2)Oc2cccc(C3CCN(Cc4nc5c(OCC6CCO6)cc(C(=O)O)nc5n4Cc4cncs4)CC3)c2O1] |
| 56 | 2-((4-(6-((4-Cyano-2- fluorobenzyl)oxy)pyridin-2- yl)piperidin-1-yl)methyl)-4- (1,1-difluoroethyl)-1- (thiazol-5-ylmethyl)-1H- benzo[d]imidazole-6- carboxylic acid | 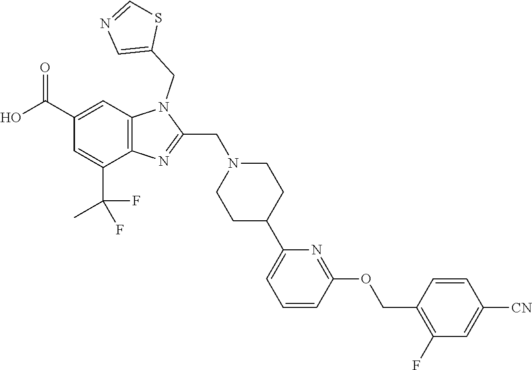 [SMILES: CC(F)(F)c1cc(C(=O)O)cc2c1nc(CN1CCC(c3cccc(OCc4ccc(C#N)cc4F)n3)CC1)n2Cc1cncs1] |
| 57 | 2-((4-((S)-2-(5- Chloropyridin-2-yl)-2- methylbenzo[d][1,3]dioxol- 4-yl)piperidin-1-yl)methyl)- 4-(1, 1-difluoroethyl)-1-(((S)- oxetan-2-yl)methyl)-1H- benzo[d]imidazole-6- carboxylic acid | 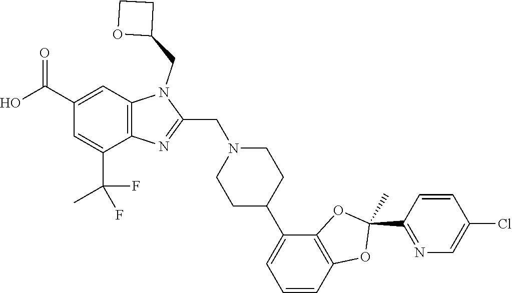 [SMILES: CC(F)(F)c1cc(C(=O)O)cc2c1nc(CN1CCC(c3cccc4c3O[C@@](C)(c3ccc(Cl)cn3)O4)CC1)n2C[C@@H]1CCO1] |
| 58 | 4-(1,1-Difluoroethyl)-2-((4- ((S)-2-(5-fluoropyridin-2-yl)- 2- methylbenzo[d][1,3]dioxol- 4-yl)piperidin-1-yl)methyl)- 1-(((S)-oxetan-2-yl)methyl)- 1H-benzo[d]imidazole-6- carboxylic acid | 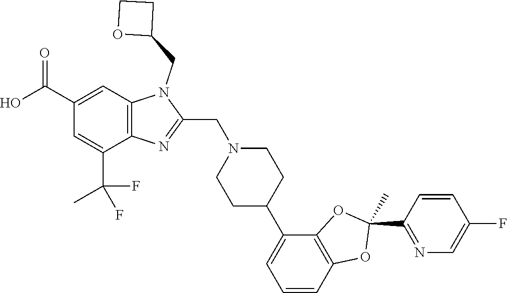 [SMILES: CC(F)(F)c1cc(C(=O)O)cc2c1nc(CN1CCC(c3cccc4c3O[C@@](C)(c3ccc(F)cn3)O4)CC1)n2C[C@@H]1CCO1] |
| 59 | 2-((4-((S)-2-(5- Cyanopyridin-2-yl)-2- methylbenzo[d][1,3]dioxol- 4-yl)piperidin-1-yl)methyl)- 4-(1, 1-difluoroethyl)-1-(((S)- oxetan-2-yl)methyl)-1H- benzo[d]imidazole-6- carboxylic acid | 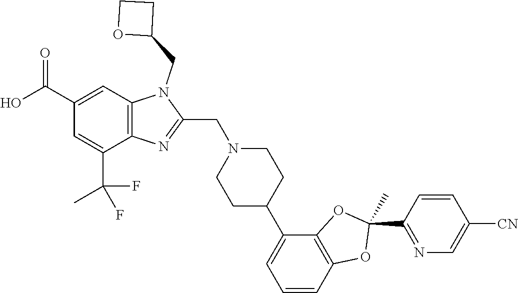 [SMILES: CC(F)(F)c1cc(C(=O)O)cc2c1nc(CN1CCC(c3cccc4c3O[C@@](C)(c3ccc(C#N)cn3)O4)CC1)n2C[C@@H]1CCO1] |
| 60 | (S)-2-((4-(2-(5- Chloropyridin-2-yl)-2- methylbenzo[d][1,3]dioxol- 4-yl)piperidin-1-yl)methyl)- 4-(1,1-difluoroethyl)-1- (thiazol-5-ylmethyl)-1H- benzo[d]imidazole-6- carboxylic acid | 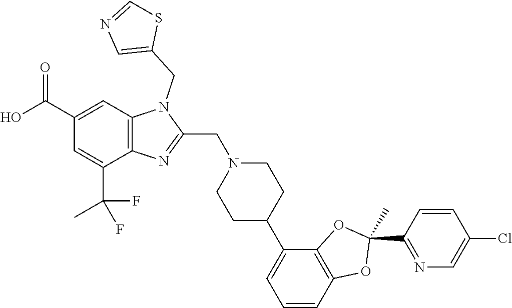 [SMILES: CC(F)(F)c1cc(C(=O)O)cc2c1nc(CN1CCC(c3cccc4c3O[C@@](C)(c3ccc(Cl)cn3)O4)CC1)n2Cc1cncs1] |
| 61 | 2-((4-((S)-2-(5- Chloropyridin-2-yl)-2- methylbenzo[d][1,3]dioxol- 4-yl)piperidin-1-yl)methyl)- 4-(1, 1-difluoroethyl)-1-((1,1- dioxidothietan-2-yl)methyl)- 1H-benzo[d]imidazole-6- carboxylic acid | 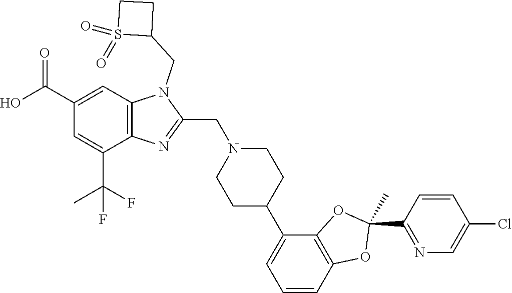 [SMILES: CC(F)(F)c1cc(C(=O)O)cc2c1nc(CN1CCC(c3cccc4c3O[C@@](C)(c3ccc(Cl)cn3)O4)CC1)n2CC1CCS1(=O)=O] |
| 62 | (S)-2-((4-(2-(5- Chloropyridin-2-yl)-2- methylbenzo[d][1,3]dioxol- 4-yl)piperidin-1-yl)methyl)- 1-((1- cyanocyclopropyl)methyl)-4- (1,1-difluoroethyl)-1H- benzo[d]imidazole-6- carboxylic acid | 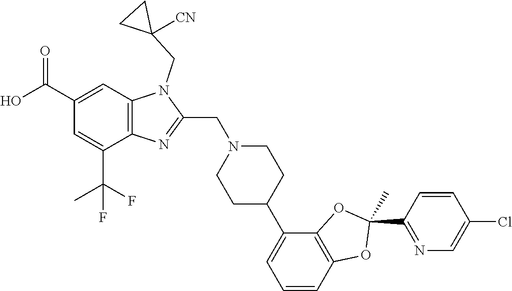 [SMILES: CC(F)(F)c1cc(C(=O)O)cc2c1nc(CN1CCC(c3cccc4c3O[C@@](C)(c3ccc(Cl)cn3)O4)CC1)n2CC1(C#N)CC1] |
| 63 | (S)-2-((4-(3-(4-Cyano-2- fluorophenyl)-2,3- dihydrobenzo[b][1,4]dioxin- 5-yl)piperidin-1-yl)methyl)- 4-methoxy-1-(thiazol-5- ylmethyl)-1H- benzo[d]imidazole-6- carboxylic acid | 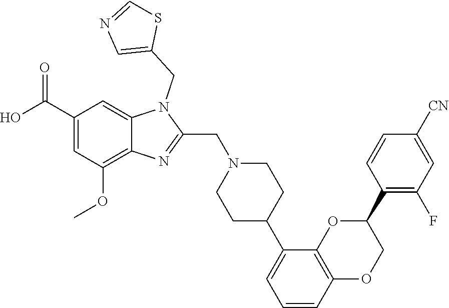 [SMILES: COc1cc(C(=O)O)cc2c1nc(CN1CCC(c3cccc4c3O[C@@H](c3ccc(C#N)cc3F)CO4)CC1)n2Cc1cncs1] |
| 64 | (R)-2-((4-(2-(4-Cyano-2- fluorophenyl)-2,3- dihydrobenzo[b][1,4]dioxin- 5-yl)piperidin-1-yl)methyl)- 4-methoxy-1-(thiazol-5- ylmethyl)-1H- benzo[d]imidazole-6- carboxylic acid | 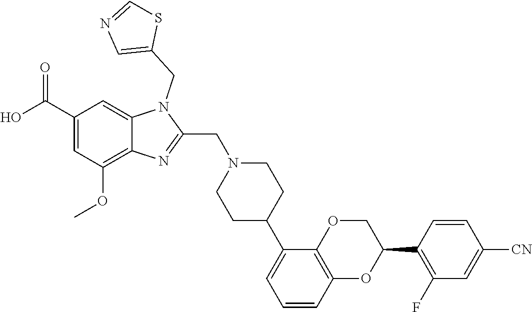 [SMILES: COc1cc(C(=O)O)cc2c1nc(CN1CCC(c3cccc4c3OC[C@@H](c3ccc(C#N)cc3F)O4)CC1)n2Cc1cncs1] |
| 65 | 2-((4-((S)-3-(4-Cyano-2- fluorophenyl)-2,3- dihydrobenzo[b][1,4]dioxin- 5-yl)piperidin-1-yl)methyl)- 4-methoxy-1-(((S)-oxetan-2- yl)methyl)-1H- benzo[d]imidazole-6- carboxylic acid | 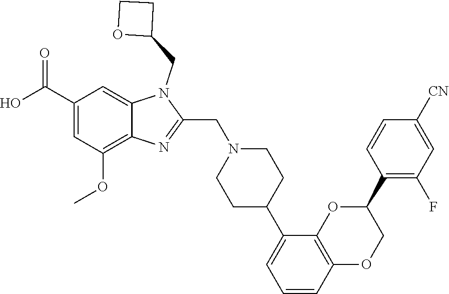 [SMILES: COc1cc(C(=O)O)cc2c1nc(CN1CCC(c3cccc4c3O[C@@H](c3ccc(C#N)cc3F)CO4)CC1)n2C[C@@H]1CCO1] |
| 66 | 2-((4-((R)-2-(4-Cyano-2- fluorophenyl)-2,3- dihydrobenzo[b][1,4]dioxin- 5-yl)piperidin-1-yl)methyl)- 4-methoxy-1-(((S)-oxetan-2- yl)methyl)-1H- benzo[d]imidazole-6- carboxylic acid | 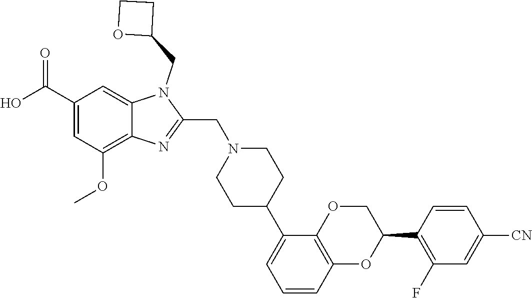 [SMILES: COc1cc(C(=O)O)cc2c1nc(CN1CCC(c3cccc4c3OC[C@@H](c3ccc(C#N)cc3F)O4)CC1)n2C[C@@H]1CCO1] |
| 67 | (S)-2-((4-(3-(4-Cyano-2- fluorophenyl)-2,3- dihydrobenzo[b][1,4]dioxin- 5-yl)piperidin-1-yl)methyl)- 4-(2-methoxyethoxy)-1- (thiazol-5-ylmethyl)-1H- benzo[d]imidazole-6- carboxylic acid | 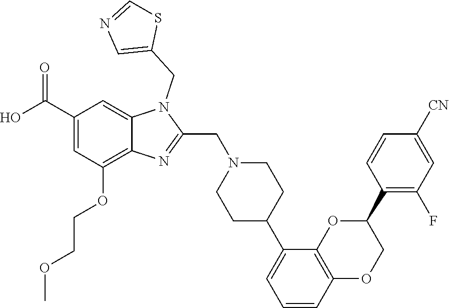 [SMILES: COCCOc1cc(C(=O)O)cc2c1nc(CN1CCC(c3cccc4c3O[C@@H](c3ccc(C#N)cc3F)CO4)CC1)n2Cc1cncs1] |
| 68 | (R)-2-((4-(2-(4-Cyano-2- fluorophenyl)-2,3- dihydrobenzo[b][1,4]dioxin- 5-yl)piperidin-1-yl)methyl)- 4-(2-methoxyethoxy)-1- (thiazol-5-ylmethyl)-1H- benzo[d]imidazole-6- carboxylic acid | 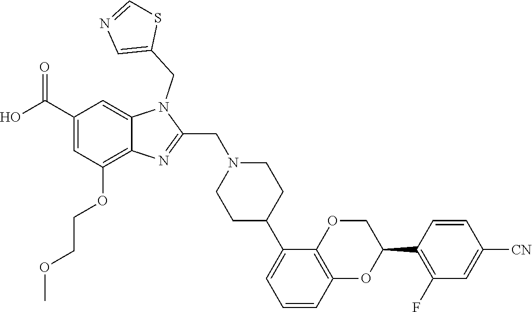 [SMILES: COCCOc1cc(C(=O)O)cc2c1nc(CN1CCC(c3cccc4c3OC[C@@H](c3ccc(C#N)cc3F)O4)CC1)n2Cc1cncs1] |
| 69 | 2-((4-((S)-3-(4-Cyano-2- fluorophenyl)-2,3- dihydrobenzo[b][1,4]dioxin- 5-yl)piperidin-1-yl)methyl)- 4-(2-methoxyethoxy)-1- (((S)-oxetan-2-yl)methyl)- 1H-benzo[d]imidazole-6- carboxylic acid | 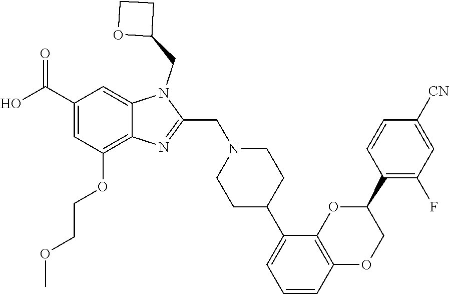 [SMILES: COCCOc1cc(C(=O)O)cc2c1nc(CN1CCC(c3cccc4c3O[C@@H](c3ccc(C#N)cc3F)CO4)CC1)n2C[C@@H]1CCO1] |
| 70 | 2-((4-((R)-2-(4-Cyano-2- fluorophenyl)-2,3- dihydrobenzo[b][1,4]dioxin- 5-yl)piperidin-1-yl)methyl)- 4-(2-methoxyethoxy)-1- (((S)-oxetan-2-yl)methyl)- 1H-benzo[d]imidazole-6- carboxylic acid | 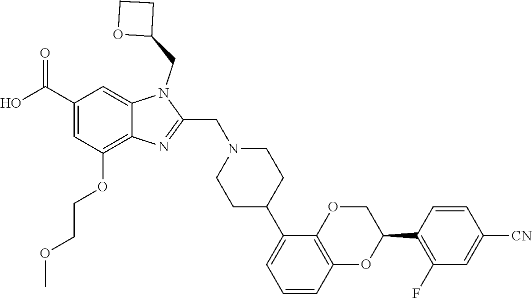 [SMILES: COCCOc1cc(C(=O)O)cc2c1nc(CN1CCC(c3cccc4c3OC[C@@H](c3ccc(C#N)cc3F)O4)CC1)n2C[C@@H]1CCO1] |
| 71 | 2-((4-((R)-2-(5- chloropyridin-2-yl)-2- methylbenzo[d][1,3]dioxol- 4-yl)piperidin-1-yl)methyl)- 4-(1,1-difluoroethyl)-1-(((S)- oxetan-2-yl)methyl)-1H- benzo[d]imidazole-6- carboxylic acid | 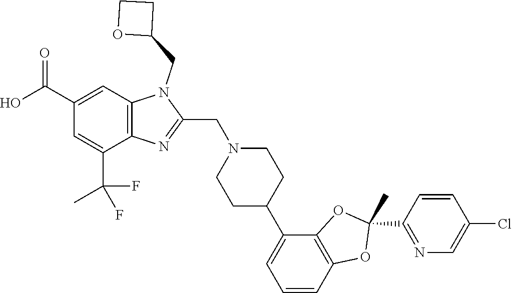 [SMILES: CC(F)(F)c1cc(C(=O)O)cc2c1nc(CN1CCC(c3cccc4c3O[C@](C)(c3ccc(Cl)cn3)O4)CC1)n2C[C@@H]1CCO1] |
| 72 | 2-((4-((S)-2-(5- chloropyridin-2-yl)-2- methylbenzo[d][1,3]dioxol- 4-yl)piperidin-1-yl)methyl)- 1-(((S)-oxetan-2-yl)methyl)- 4-(oxetan-3-yloxy)-1H- benzo[d]imidazole-6- carboxylic acid | 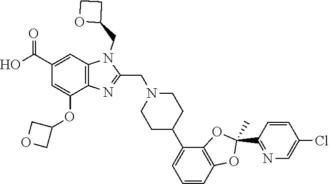 [SMILES: C[C@]1(c2ccc(Cl)cn2)Oc2cccc(C3CCN(Cc4nc5c(OC6COC6)cc(C(=O)O)cc5n4C[C@@H]4CCO4)CC3)c2O1] |
| 73 | 2-((4-(6-((4-cyano-2- fluorobenzyl)oxy)pyridin-2- yl)piperidin-1-yl)methyl)-4- isopropoxy-1-(thiazol-5- ylmethyl)-1H- benzo[d]imidazole-6- carboxylic acid | 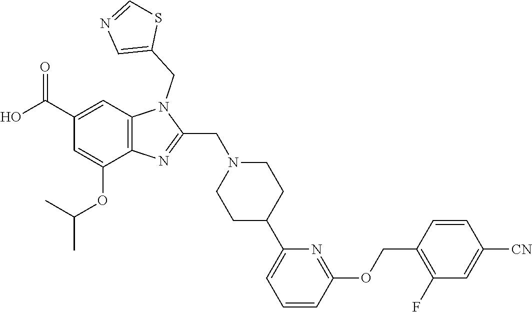 [SMILES: CC(C)Oc1cc(C(=O)O)cc2c1nc(CN1CCC(c3cccc(OCc4ccc(C#N)cc4F)n3)CC1)n2Cc1cncs1] |
| 74 | (S)-2-((4-(2-(5- chloropyridin-2-yl)-2- methylbenzo[d][1,3]dioxol- 4-yl)piperidin-1-yl)methyl)- 4-(2-methoxyethoxy)-1H- benzo[d]imidazole-6- carboxylic acid | 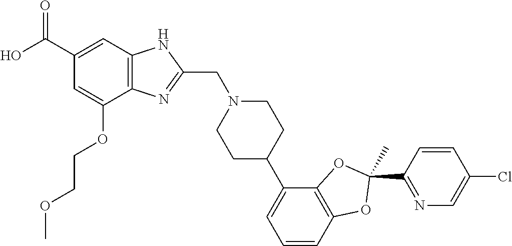 [SMILES: COCCOc1cc(C(=O)O)cc2[nH]c(CN3CCC(c4cccc5c4O[C@@](C)(c4ccc(Cl)cn4)O5)CC3)nc12] |
| 75 | (S)-2-((4-(2-(5- chloropyridin-2-yl)-2- methylbenzo[d][1,3]dioxol- 4-yl)piperidin-1-yl)methyl)- 3,4-dihydro-5-oxa-1,2a- diazaacenaphthylene-7- carboxylic acid | 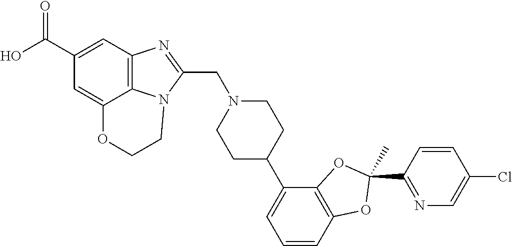 [SMILES: C[C@]1(c2ccc(Cl)cn2)Oc2cccc(C3CCN(Cc4nc5cc(C(=O)O)cc6c5n4CCO6)CC3)c2O1] |
| 76 | (S)-2-((4-(2-(5- chloropyridin-2-yl)-2- methylbenzo[d][1,3]dioxol- 4-yl)piperidin-1-yl)methyl)- 7-(2-methoxyethoxy)-1- methyl-1H- benzo[d]imidazole-5- carboxylic acid | 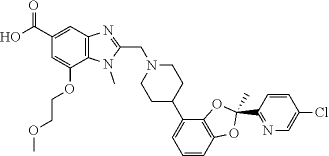 [SMILES: COCCOc1cc(C(=O)O)cc2nc(CN3CCC(c4cccc5c4O[C@@](C)(c4ccc(Cl)cn4)O5)CC3)n(C)c12] |
| 77 | 2-((4-((S)-2-(5- chloropyridin-2-yl)-2- methylbenzo[d][1,3]dioxol- 4-yl)piperidin-1-yl)methyl)- 1-(((S)-oxetan-2-yl)methyl)- 4-(oxetan-3-ylmethoxy)-1H- benzo[d]imidazole-6- carboxylic acid | 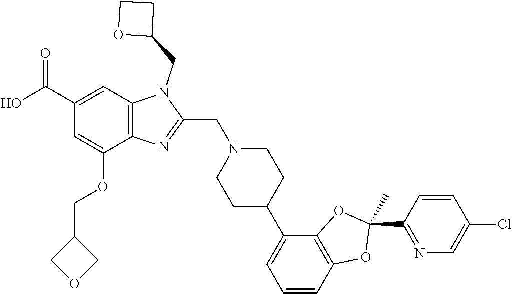 [SMILES: C[C@]1(c2ccc(Cl)cn2)Oc2cccc(C3CCN(Cc4nc5c(OCC6COC6)cc(C(=O)O)cc5n4C[C@@H]4CCO4)CC3)c2O1] |
| 78 | 2-((4-((S)-2-(5- chloropyridin-2-yl)-2- methylbenzo[d][1,3]dioxol- 4-yl)piperidin-1-yl)methyl)- 4-(difluoromethoxy)-1-(((S)- oxetan-2-yl)methyl)-1H- benzo[d]imidazole-6- carboxylic acid | 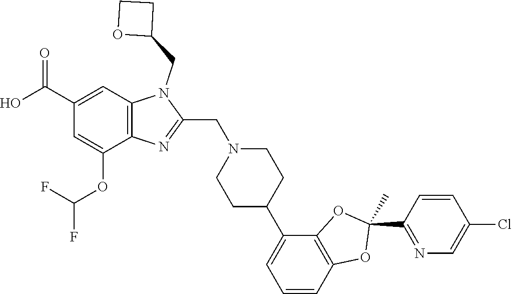 [SMILES: C[C@]1(c2ccc(Cl)cn2)Oc2cccc(C3CCN(Cc4nc5c(OC(F)F)cc(C(=O)O)cc5n4C[C@@H]4CCO4)CC3)c2O1] |
| 79 | 2-(((1R,4R)-4-((S)-2-(5- chloropyridin-2-yl)-2- methylbenzo[d][1,3]dioxol- 4-yl)cyclohexyl) (methyl)amino)- 4-methoxy-1-(((S)-oxetan- 2-yl)methyl)-1H- benzo[d]imidazole-6- carboxylic acid | 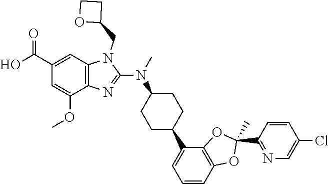 [SMILES: COc1cc(C(=O)O)cc2c1nc(N(C)[C@H]1CC[C@@H](c3cccc4c3O[C@@](C)(c3ccc(Cl)cn3)O4)CC1)n2C[C@@H]1CCO1] |
| 80 | 2-(((1S,4S)-4-((S)-2-(5- chloropyridin-2-yl)-2- methylbenzo[d][1,3]dioxol- 4-yl)cyclohexyl) (methyl)amino)-4- methoxy-1-(((S)-oxetan- 2-yl)methyl)-1H- benzo[d]imidazole-6- carboxylic acid | 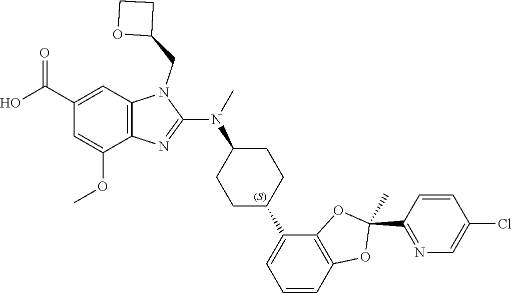 |
| 81 | 2-((4-((S)-2-(5- chloropyridin-2-yl)-2- methylbenzo[d][1,3]dioxol- 4-yl)piperidin-1-yl)methyl)- 4-(2-fluoroethoxy)-1-(((S)- oxetan-2-yl)methyl)-1H- benzo[d]imidazole-6- carboxylic acid | 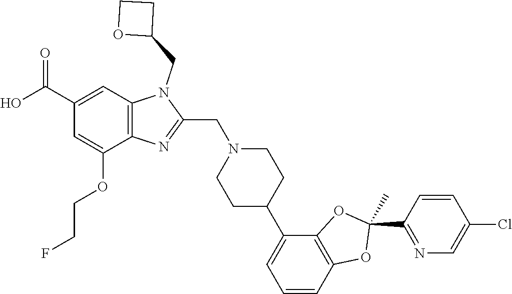 [SMILES: C[C@]1(c2ccc(Cl)cn2)Oc2cccc(C3CCN(Cc4nc5c(OCCF)cc(C(=O)O)cc5n4C[C@@H]4CCO4)CC3)c2O1] |
| 82 | 2-((4-(6-((4-cyano-2- fluorobenzyl)oxy)pyridin-2- yl)piperidin-1-yl)methyl)-4- hydroxy-1-(thiazol-5- ylmethyl)-1H- benzo[d]imidazole-6- carboxylic acid | 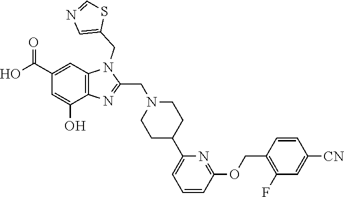 [SMILES: N#Cc1ccc(COc2cccc(C3CCN(Cc4nc5c(O)cc(C(=O)O)cc5n4Cc4cncs4)CC3)n2)c(F)c1] |
| 83 | (S)-2-((4-(2-(5- chloropyridin-2-yl)-2- methylbenzo[d][1,3]dioxol- 4-yl)piperidin-1-yl)methyl)- 4-(difluoromethoxy)-1- methyl-1H- benzo[d]imidazole-6- carboxylic acid | 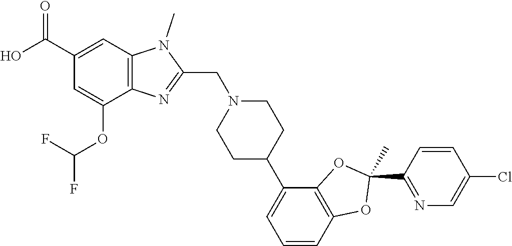 [SMILES: Cn1c(CN2CCC(c3cccc4c3O[C@@](C)(c3ccc(Cl)cn3)O4)CC2)nc2c(OC(F)F)cc(C(=O)O)cc21] |
| 84 | (S)-2-((4-(2-(5- chloropyridin-2-yl)-2- methylbenzo[d][1,3]dioxol- 4-yl)piperidin-1-yl)methyl)- 4-isopropoxy-1-methyl-1H- benzo[d]imidazole-6- carboxylic acid | 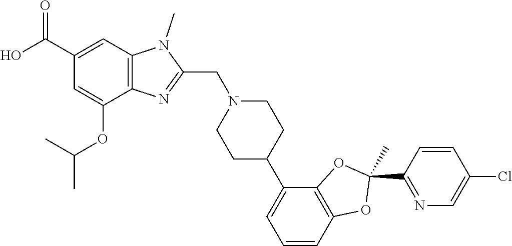 [SMILES: CC(C)Oc1cc(C(=O)O)cc2c1nc(CN1CCC(c3cccc4c3O[C@@](C)(c3ccc(Cl)cn3)O4)CC1)n2C] |
| 85 | (S)-2-((4-(2-(5- chloropyridin-2-yl)-2- methylbenzo[d][1,3]dioxol- 4-yl)piperidin-1-yl)methyl)- 4-methoxy-1-methyl-1H- benzo[d]imidazole-6- carboxylic acid | 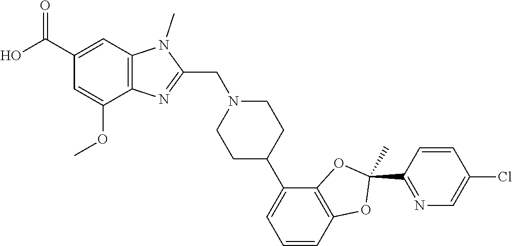 [SMILES: COc1cc(C(=O)O)cc2c1nc(CN1CCC(c3cccc4c3O[C@@](C)(c3ccc(Cl)cn3)O4)CC1)n2C] |
| 86 | (S)-2-((4-(2-(5- chloropyridin-2-yl)-2- methylbenzo[d][1,3]dioxol- 4-yl)piperidin-1-yl)methyl)- 1-isopropyl-4-methoxy-1H- benzo[d]imidazole-6- carboxylic acid | 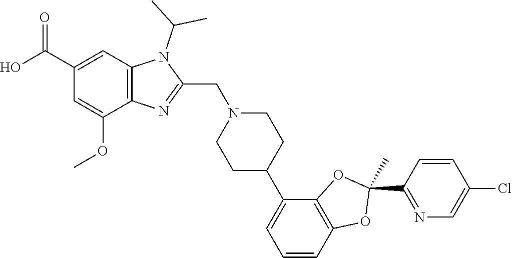 [SMILES: COc1cc(C(=O)O)cc2c1nc(CN1CCC(c3cccc4c3O[C@@](C)(c3ccc(Cl)cn3)O4)CC1)n2C(C)C] |
| 87 | (S)-2-((4-(2-(5- chloropyridin-2-yl)-2- methylbenzo[d][1,3]dioxol- 4-yl)piperidin-1-yl)methyl)- 4-(1,1-difluoroethyl)-1- methyl-1H- benzo[d]imidazole-6- carboxylic acid | 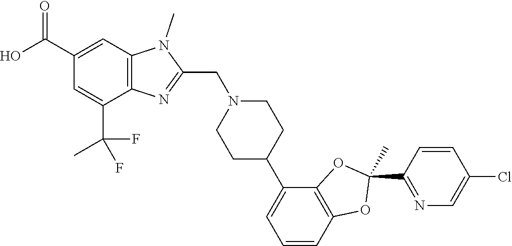 [SMILES: Cn1c(CN2CCC(c3cccc4c3O[C@@](C)(c3ccc(Cl)cn3)O4)CC2)nc2c(C(C)(F)F)cc(C(=O)O)cc21] |
| 88 | (S)-2-((4-(2-(5- chloropyridin-2-yl)-2- methylbenzo[d][1,3]dioxol- 4-yl)piperidin-1-yl)methyl)- 1-ethyl-4-isopropoxy-1H- benzo[d]imidazole-6- carboxylic acid | 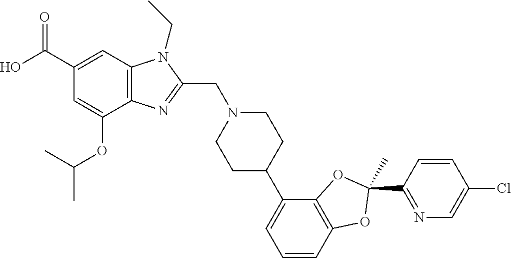 [SMILES: CCn1c(CN2CCC(c3cccc4c3O[C@@](C)(c3ccc(Cl)cn3)O4)CC2)nc2c(OC(C)C)cc(C(=O)O)cc21] |
| 89 | 2-(((15,5R,6S)-5-((S)-2-(5- chloropyridin-2-yl)-2- methylbenzo[d][1,3]dioxol- 4-yl)-2- azabicyclo[4.1.0]heptan-2- yl)methyl)-4-methoxy-1- (((S)-oxetan-2-yl)methyl)- 1H-benzo[d]imidazole-6- carboxylic acid | 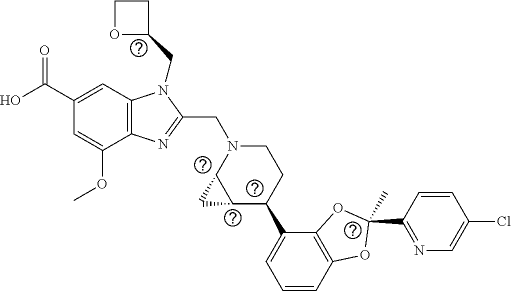 [SMILES: COc1cc(C(=O)O)cc2c1nc(CN1CC[C@@H](c3cccc4c3O[C@@](C)(c3ccc(Cl)cn3)O4)[C@@H]3C[C@@H]31)n2C[C@@H]1CCO1] |
| 90 | (S)-2-((4-(2-(5- chloropyridin-2-yl)-2- methylbenzo[d][1,3]dioxol- 4-yl)piperidin-1-yl)methyl)- 4-hydroxy-1-(thiazol-5- ylmethyl)-1H- benzo[d]imidazole-6- carboxylic acid | 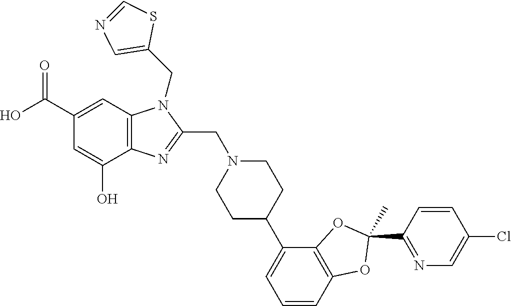 [SMILES: C[C@]1(c2ccc(Cl)cn2)Oc2cccc(C3CCN(Cc4nc5c(O)cc(C(=O)O)cc5n4Cc4cncs4)CC3)c2O1] |
| 91 | (R)-2-((4-(3-(4-cyano-2- fluorophenyl)-2,3- dihydrobenzo[b][1,4]dioxin- 5-yl)piperidin-1-yl)methyl)- 4-methoxy-1-(thiazol-5- ylmethyl)-1H- benzo[d]imidazole-6- carboxylic acid | 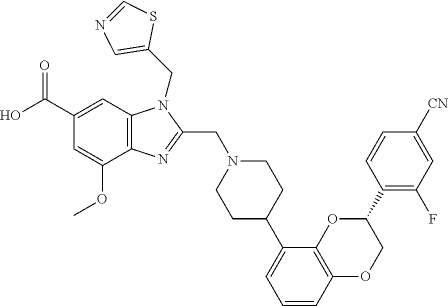 [SMILES: COc1cc(C(=O)O)cc2c1nc(CN1CCC(c3cccc4c3O[C@H](c3ccc(C#N)cc3F)CO4)CC1)n2Cc1cncs1] |
| 92 | 2-((4-((S)-2-(5- chloropyridin-2-yl)-2- methylbenzo[d][1,3]dioxol- 4-yl)piperidin-1-yl)methyl)- 4-hydroxy-1-(((S)-oxetan-2- yl)methyl)-1H- benzo[d]imidazole-6- carboxylic acid | 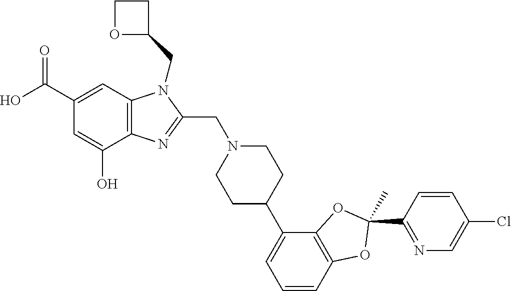 [SMILES: C[C@]1(c2ccc(Cl)cn2)Oc2cccc(C3CCN(Cc4nc5c(O)cc(C(=O)O)cc5n4C[C@@H]4CCO4)CC3)c2O1] |
| 93 | 2-((4-((S)-2-(5- chloropyridin-2-yl)-2- methylbenzo[d][1,3]dioxol- 4-yl)piperidin-1-yl)methyl)- 3-(((S)-oxetan-2-yl)methyl)- 7,8-dihydro-3H- [1,4]dioxino[2′,3′:3,4]benzo [1,2-d]imidazole-5-carboxylic acid | 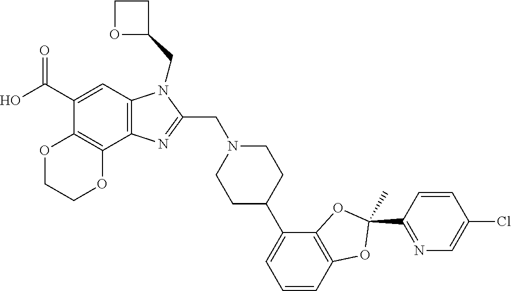 [SMILES: C[C@]1(c2ccc(Cl)cn2)Oc2cccc(C3CCN(Cc4nc5c6c(c(C(=O)O)cc5n4C[C@@H]4CCO4)OCCO6)CC3)c2O1] |
| 94 | 2-((R)-1-(4-(6-((4-cyano-2- fluorobenzyl)oxy)pyridin-2- yl)piperidin-1-yl)ethyl)-4- methoxy-1-(((S)-oxetan-2- yl)methyl)-1H- benzo[d]imidazole-6- carboxylic acid | 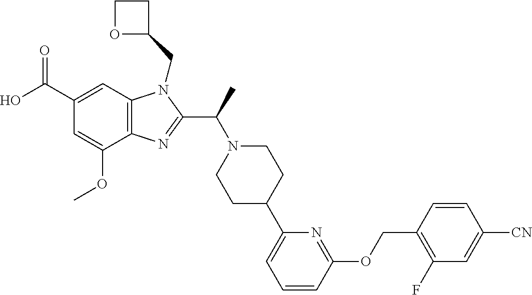 [SMILES: COc1cc(C(=O)O)cc2c1nc([C@@H](C)N1CCC(c3cccc(OCc4ccc(C#N)cc4F)n3)CC1)n2C[C@@H]1CCO1] |
| 95 | 2-((S)-1-(4-(6-((4-cyano-2- fluorobenzyl)oxy)pyridin-2- yl)piperidin-1-yl)ethyl)-4- methoxy-1-(((S)-oxetan-2- yl)methyl)-1H- benzo[d]imidazole-6- carboxylic acid | 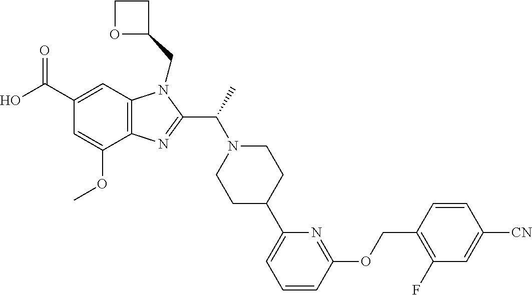 [SMILES: COc1cc(C(=O)O)cc2c1nc([C@H](C)N1CCC(c3cccc(OCc4ccc(C#N)cc4F)n3)CC1)n2C[C@@H]1CCO1] |
| 96 | 2-((S)-1-(4-((S)-2-(5- chloropyridin-2-yl)-2- methylbenzo[d][1,3]dioxol- 4-yl)piperidin-1-yl)ethyl)-4- methoxy-1-methyl-1H- benzo[d]imidazole-6- carboxylic acid | 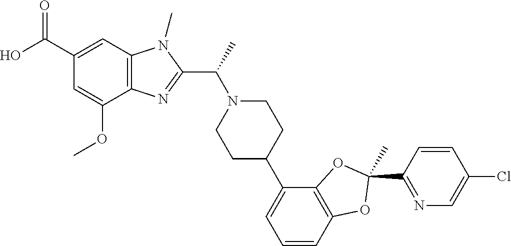 [SMILES: COc1cc(C(=O)O)cc2c1nc([C@H](C)N1CCC(c3cccc4c3O[C@@](C)(c3ccc(Cl)cn3)O4)CC1)n2C] |
| 97 | 2-((R)-1-(4-((S)-2-(5- chloropyridin-2-yl)-2- methylbenzo[d][1,3]dioxol- 4-yl)piperidin-1-yl)ethyl)-4- methoxy-1-methyl-1H- benzo[d]imidazole-6- carboxylic acid | 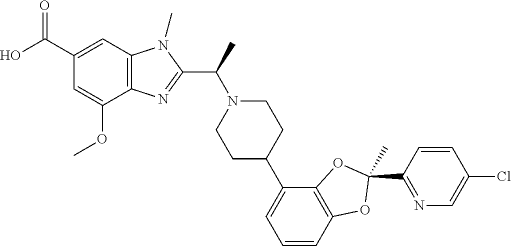 [SMILES: COc1cc(C(=O)O)cc2c1nc([C@@H](C)N1CCC(c3cccc4c3O[C@@](C)(c3ccc(Cl)cn3)O4)CC1)n2C] |
| 98 | 2-((4-((S)-2-(5- chloropyridin-2-yl)-2- methylbenzo[d][1,3]dioxol- 4-yl)piperidin-1-yl)methyl)- 4-(methoxy-d3)-1-(((S)- oxetan-2-yl)methyl)-1H- benzo[d]imidazole-6- carboxylic acid | 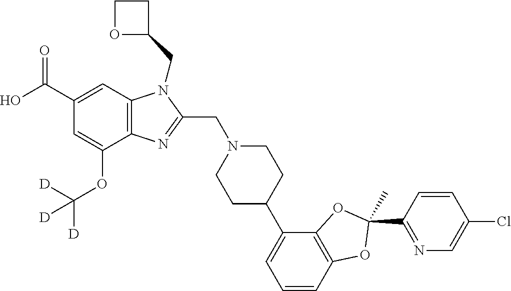 [SMILES: [2H]C([2H])([2H])Oc1cc(C(=O)O)cc2c1nc(CN1CCC(c3cccc4c3O[C@@](C)(c3ccc(Cl)cn3)O4)CC1)n2C[C@@H]1CCO1] |
| 99 | 2-((4-((S)-2-(5- chloropyridin-2-yl)-2- methylbenzo[d][1,3]dioxol- 4-yl)piperidin-1-yl)methyl)- 1-(((S)-oxetan-2-yl)methyl)- 7,8-dihydro-1H- [1,4]dioxino[2′,3′:3,4]benzo [1,2-d]imidazole-5- carboxylic acid | 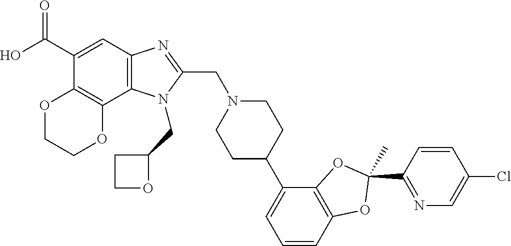 [SMILES: C[C@]1(c2ccc(Cl)cn2)Oc2cccc(C3CCN(Cc4nc5cc(C(=O)O)c6c(c5n4C[C@@H]4CCO4)OCCO6)CC3)c2O1] |
| 100 | (S)-2-((4-(6-((4-cyano-2- fluorobenzyl)oxy)pyridin-2- yl)piperidin-1-yl)methyl)-3- (oxetan-2-ylmethyl)-7,8- dihydro-3H- [1,4]dioxino[2′,3′:3,4]benzo [1,2-d]imidazole-5-carboxylic acid | 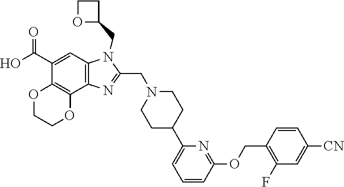 [SMILES: N#Cc1ccc(COc2cccc(C3CCN(Cc4nc5c6c(c(C(=O)O)cc5n4C[C@@H]4CCO4)OCCO6)CC3)n2)c(F)c1] |
| 101 | (S)-2-((4-(6-((4-cyano-2- fluorobenzyl)oxy)pyridin-2- yl)piperidin-1-yl)methyl)-1- (oxetan-2-ylmethyl)-7,8- dihydro-1H- [1,4]dioxino[2′,3′:3,4]benzo [1,2-d]imidazole-5-carboxylic acid | 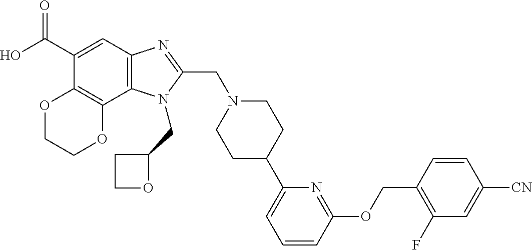 [SMILES: N#Cc1ccc(COc2cccc(C3CCN(Cc4nc5cc(C(=O)O)c6c(c5n4C[C@@H]4CCO4)OCCO6)CC3)n2)c(F)c1] |
| 102 | (S)-2-(1-(4-(6-((4-cyano-2- fluorobenzyl)oxy)pyridin-2- yl)piperidin-1-yl)ethyl)-4- methoxy-1-methyl-1H- benzo[d]imidazole-6- carboxylic acid | 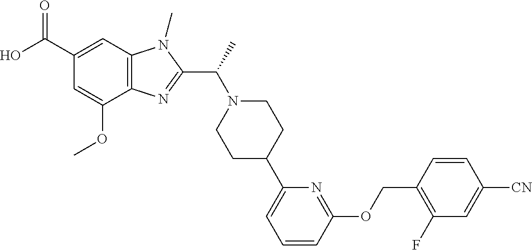 [SMILES: COc1cc(C(=O)O)cc2c1nc([C@H](C)N1CCC(c3cccc(OCc4ccc(C#N)cc4F)n3)CC1)n2C] |
| 103 | (R)-2-(1-(4-(6-((4-cyano-2- fluorobenzyl)oxy)pyridin-2- yl)piperidin-1-yl)ethyl)-4- methoxy-1-methyl-1H- benzo[d]imidazole-6- carboxylic acid | 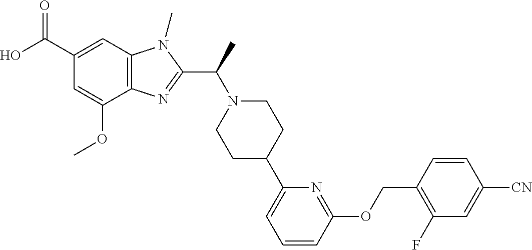 [SMILES: COc1cc(C(=O)O)cc2c1nc([C@@H](C)N1CCC(c3cccc(OCc4ccc(C#N)cc4F)n3)CC1)n2C] |
| 104 | 2-((4-(6-((4-cyano-2- fluorobenzyl)oxy)pyridin-2- yl)piperidin-1-yl)methyl)- 3,4-dihydro-5-oxa-1,2a- diazaacenaphthylene-6- carboxylic acid | 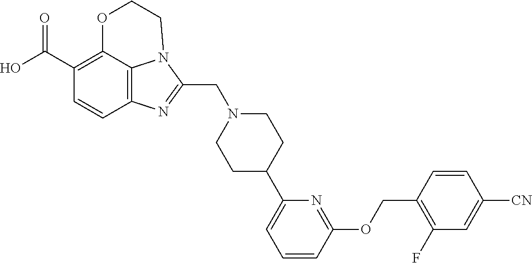 [SMILES: N#Cc1ccc(COc2cccc(C3CCN(Cc4nc5ccc(C(=O)O)c6c5n4CCO6)CC3)n2)c(F)c1] |
| 105 | 2-((4-(6-((4-cyano-2- fluorobenzyl)oxy)pyridin-2- yl)piperidin-1-yl)methyl)-1- ((1- cyanocyclopropyl)methyl)-4- (difluoromethoxy)-1H- benzo[d]imidazole-6- carboxylic acid | 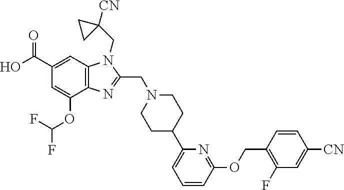 [SMILES: N#Cc1ccc(COc2cccc(C3CCN(Cc4nc5c(OC(F)F)cc(C(=O)O)cc5n4CC4(C#N)CC4)CC3)n2)c(F)c1] |
| 106 | 2-((4-(6-((4-cyano-2- fluorobenzyl)oxy)pyridin-2- yl)piperidin-1-yl)methyl)-4- (difluoromethoxy)-1-(2- methoxyethyl)-1H- benzo[d]imidazole-6- carboxylic acid | 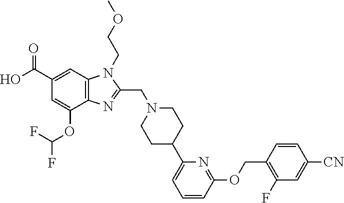 [SMILES: COCCn1c(CN2CCC(c3cccc(OCc4ccc(C#N)cc4F)n3)CC2)nc2c(OC(F)F)cc(C(=O)O)cc21] |
| 107 | 2-((4-(6-((4-cyano-2- fluorobenzyl)oxy)pyridin-2- yl)piperidin-1-yl)methyl)-4- (2-methoxyethyl)-1-methyl- 1H-imidazo[4,5-c]pyridine- 6-carboxylic acid | 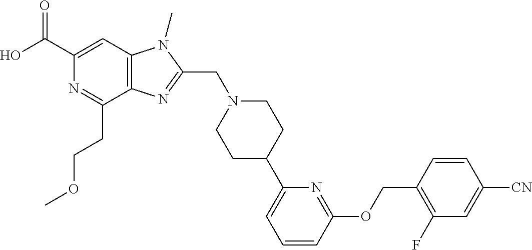 [SMILES: COCCc1nc(C(=O)O)cc2c1nc(CN1CCC(c3cccc(OCc4ccc(C#N)cc4F)n3)CC1)n2C] |
| 108 | (S)-2-((4-(6-((4-cyano-2- fluorobenzyl)oxy)pyridin-2- yl)piperidin-1-yl)methyl)-4- (methoxy-d3)-1-(oxetan-2- ylmethyl)-1H- benzo[d]imidazole-6- carboxylic acid | 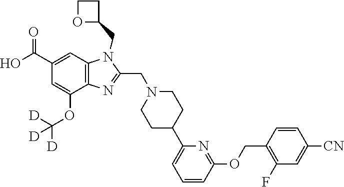 [SMILES: [2H]C([2H])([2H])Oc1cc(C(=O)O)cc2c1nc(CN1CCC(c3cccc(OCc4ccc(C#N)cc4F)n3)CC1)n2C[C@@H]1CCO1] |
| 109 | (S)-4-(tert-butoxy)-2-((4-(6- ((4-cyano-2- fluorobenzyl)oxy)pyridin-2- yl)piperidin-1-yl)methyl)-1- (oxetan-2-ylmethyl)-1H- benzo[d]imidazole-6- carboxylic acid | 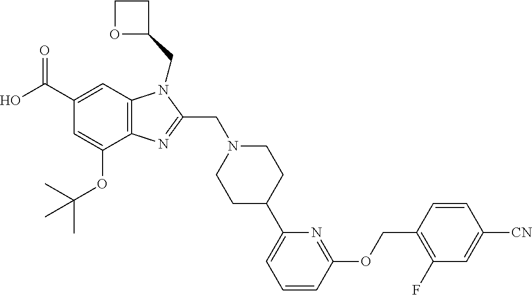 [SMILES: CC(C)(C)Oc1cc(C(=O)O)cc2c1nc(CN1CCC(c3cccc(OCc4ccc(C#N)cc4F)n3)CC1)n2C[C@@H]1CCO1] |
| 110 | (S)-2-((4-(6-((4-cyano-2- fluorobenzyl)oxy)pyridin-2- yl)piperidin-1-yl)methyl)-4- (difluoromethoxy)-1-((1,1- dioxidothietan-2-yl)methyl)- 1H-benzo[d]imidazole-6- carboxylic acid | 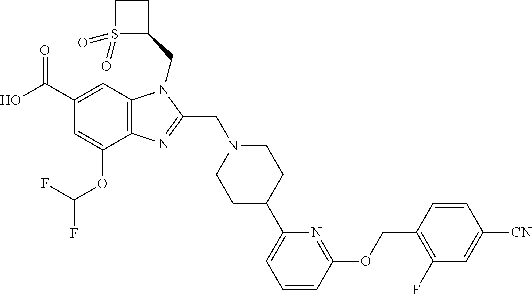 [SMILES: N#Cc1ccc(COc2cccc(C3CCN(Cc4nc5c(OC(F)F)cc(C(=O)O)cc5n4C[C@@H]4CCS4(=O)=O)CC3)n2)c(F)c1] |
| 111 | 2-((4-(3-((4-cyano-2- fluorobenzyl)oxy)phenyl) piperidin-1-yl)methyl)-4- (difluoromethoxy)-1-methyl- 1H-benzo[d]imidazole-6- carboxylic acid | 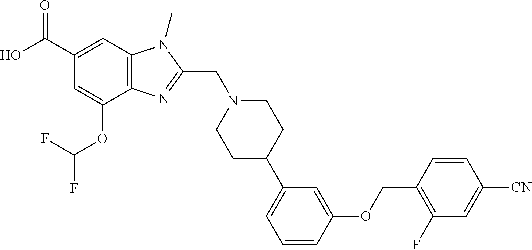 [SMILES: Cn1c(CN2CCC(c3cccc(OCc4ccc(C#N)cc4F)c3)CC2)nc2c(OC(F)F)cc(C(=O)O)cc21] |
| 112 | 2-((4-(6-((4-cyano-2- fluorobenzyl)oxy)pyridin-2- yl)piperidin-1-yl)methyl)-1- methyl-4-morpholino-1H- benzo[d]imidazole-6- carboxylic acid | 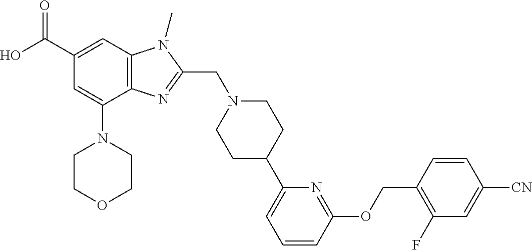 [SMILES: Cn1c(CN2CCC(c3cccc(OCc4ccc(C#N)cc4F)n3)CC2)nc2c(N3CCOCC3)cc(C(=O)O)cc21] |
| 113 | 2-((4-(6-((4-cyano-2- fluorobenzyl)oxy)pyridin-2- yl)piperidin-1-yl)methyl)-4- (fluoromethoxy)-1-methyl- 1H-benzo[d]imidazole-6- carboxylic acid | 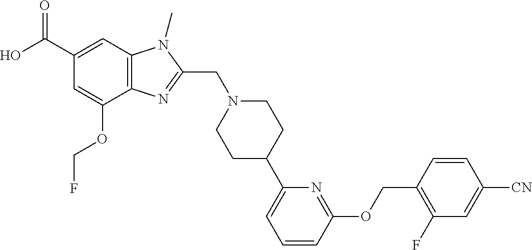 [SMILES: Cn1c(CN2CCC(c3cccc(OCc4ccc(C#N)cc4F)n3)CC2)nc2c(OCF)cc(C(=O)O)cc21] |
| 114 | 2-((4-(6-((4-cyano-2- fluorobenzyl)oxy)pyridin-2- yl)piperidin-1-yl)methyl)-4- hydroxy-1-methyl-1H- benzo[d]imidazole-6- carboxylic acid | 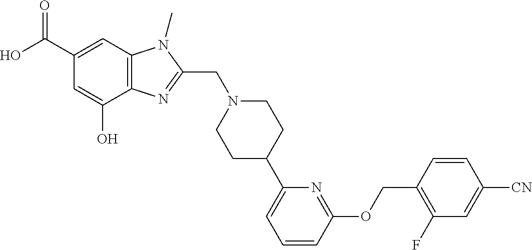 [SMILES: Cn1c(CN2CCC(c3cccc(OCc4ccc(C#N)cc4F)n3)CC2)nc2c(O)cc(C(=O)O)cc21] |
| 115 | 4-amino-2-((4-(6-((4-cyano- 2-fluorobenzyl)oxy)pyridin- 2-yl)piperidin-1-yl)methyl)- 1-methyl-1H- benzo[d]imidazole-6- carboxylic acid | 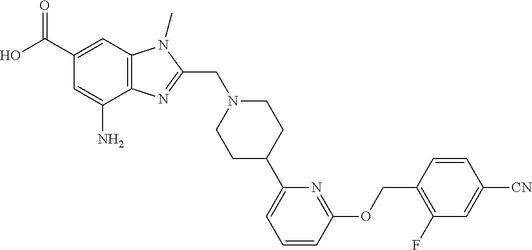 [SMILES: Cn1c(CN2CCC(c3cccc(OCc4ccc(C#N)cc4F)n3)CC2)nc2c(N)cc(C(=O)O)cc21] |
| 116 | 4-acetamido-2-((4-(6-((4- cyano-2- fluorobenzyl)oxy)pyridin-2- yl)piperidin-1-yl)methyl)-1- methyl-1H- benzo[d]imidazole-6- carboxylic acid | 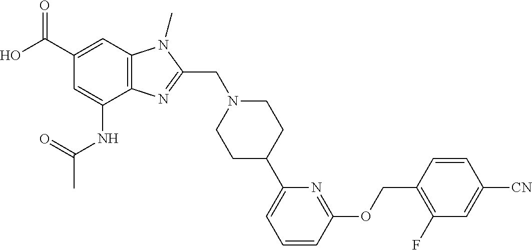 [SMILES: CC(=O)Nc1cc(C(=O)O)cc2c1nc(CN1CCC(c3cccc(OCc4ccc(C#N)cc4F)n3)CC1)n2C] |
| 117 | (S)-2-((4-(6-((4-cyano-2- fluorobenzyl)oxy)pyridin-2- yl)piperidin-1-yl)methyl)-7- fluoro-4-methoxy-1-(oxetan- 2-ylmethyl)-1H- benzo[d]imidazole-6- carboxylic acid | 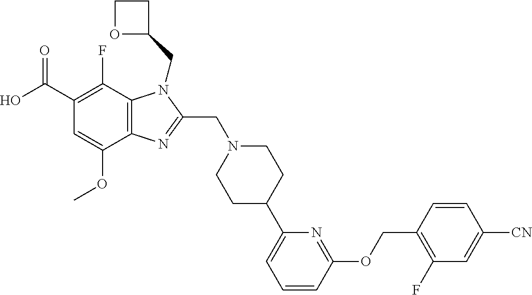 [SMILES: COc1cc(C(=O)O)c(F)c2c1nc(CN1CCC(c3cccc(OCc4ccc(C#N)cc4F)n3)CC1)n2C[C@@H]1CCO1] |
| 118 | 2-((4-(6-((4-cyano-2- fluorophenyl)methoxy- d2)pyridin-2-yl)piperidin-1- yl)methyl)-4- (difluoromethoxy)-1-methyl- 1H-benzo[d]imidazole-6- carboxylic acid | 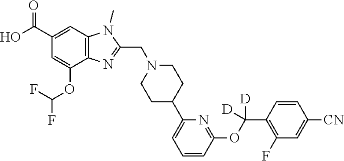 [SMILES: [2H]C([2H])(Oc1cccc(C2CCN(Cc3nc4c(OC(F)F)cc(C(=O)O)cc4n3C)CC2)n1)c1ccc(C#N)cc1F] |
| 119 | (S)-2-(1-(4-(6-((4-cyano-2- fluorobenzyl)oxy)pyridin-2- yl)piperidin-1-yl)ethyl)-4- (difluoromethoxy)-1-methyl- 1H-benzo[d]imidazole-6- carboxylic acid | 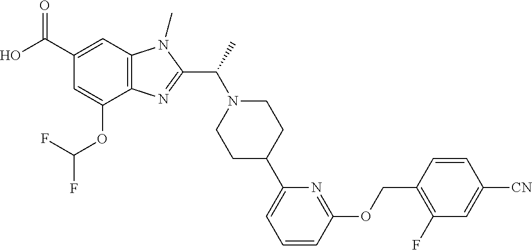 [SMILES: C[C@@H](c1nc2c(OC(F)F)cc(C(=O)O)cc2n1C)N1CCC(c2cccc(OCc3ccc(C#N)cc3F)n2)CC1] |
| 120 | 2-(4-(6-((4-cyano-2- fluorobenzyl)oxy)pyridin-2- yl)-2-fluorobenzyl)-4- (difluoromethoxy)-1-methyl- 1H-benzo[d]imidazole-6- carboxylic acid | 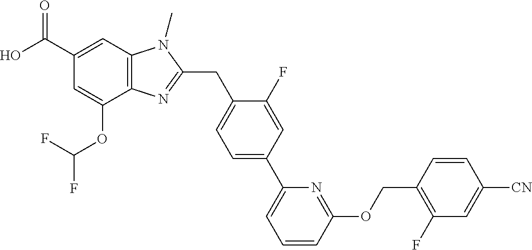 [SMILES: Cn1c(Cc2ccc(-c3cccc(OCc4ccc(C#N)cc4F)n3)cc2F)nc2c(OC(F)F)cc(C(=O)O)cc21] |
| 121 | 2-((4-(6-((4-cyano-2- fluorobenzyl)oxy)pyridin-2- yl)piperidin-1-yl)methyl)-1- methyl-4-(2,2,2- trifluoroethoxy)-1H- benzo[d]imidazole-6- carboxylic acid | 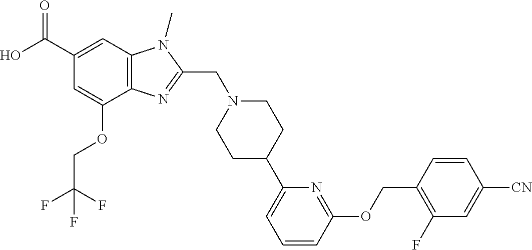 [SMILES: Cn1c(CN2CCC(c3cccc(OCc4ccc(C#N)cc4F)n3)CC2)nc2c(OCC(F)(F)F)cc(C(=O)O)cc21] |
| 122 | (R)-2-(1-(4-(6-((4-cyano-2- fluorobenzyl)oxy)pyridin-2- yl)piperidin-1-yl)ethyl)-4- (difluoromethoxy)-1-methyl- 1H-benzo[d]imidazole-6- carboxylic acid | 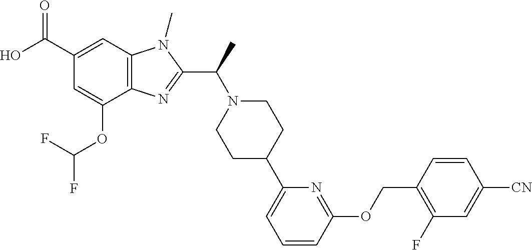 [SMILES: C[C@H](c1nc2c(OC(F)F)cc(C(=O)O)cc2n1C)N1CCC(c2cccc(OCc3ccc(C#N)cc3F)n2)CC1] |
| 123 | (S)-2-((4-(3-(4-cyano-2- fluorophenyl)-2,3- dihydrobenzo[b][1,4]dioxin- 5-yl)piperidin-1-yl)methyl)- 4-(difluoromethoxy)-1- methyl-1H- benzo[d]imidazole-6- carboxylic acid | 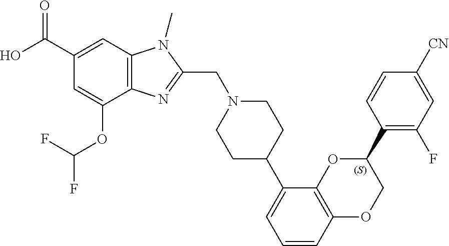 |
| 124 | (R)-2-((4-(3-(4-cyano-2- fluorophenyl)-2,3- dihydrobenzo[b][1,4]dioxin- 5-yl)piperidin-1-yl)methyl)- 4-(difluoromethoxy)-1- methyl-1H- benzo[d]imidazole-6- carboxylic acid | 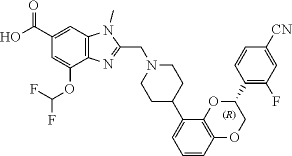 |
| 125 | 2-((4-(6-((4-cyano-2- fluorobenzyl)oxy)pyridin-2- yl)piperidin-1-yl)methyl)-1- methyl-4-(trifluoromethoxy)- 1H-benzo[d]imidazole-6- carboxylic acid | 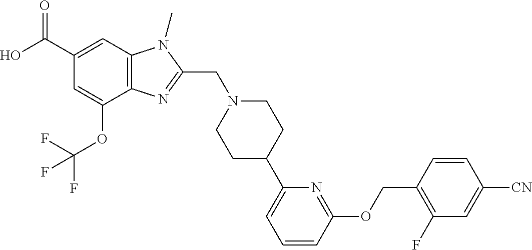 [SMILES: Cn1c(CN2CCC(c3cccc(OCc4ccc(C#N)cc4F)n3)CC2)nc2c(OC(F)(F)F)cc(C(=O)O)cc21] |
| 126 | 2-((4-(6-((4-cyano-2- fluorobenzyl)oxy)pyridin-2- yl)piperidin-1-yl)methyl)-4- (2-hydroxypropan-2-yl)-1- methyl-1H- benzo[d]imidazole-6- carboxylic acid | 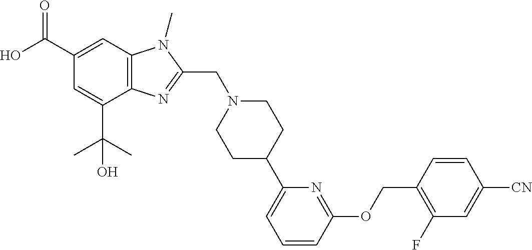 [SMILES: Cn1c(CN2CCC(c3cccc(OCc4ccc(C#N)cc4F)n3)CC2)nc2c(C(C)(C)O)cc(C(=O)O)cc21] |
| 127 | 2-((4-(6-((4-cyano-2- fluorobenzyl)oxy)pyridin-2- yl)piperidin-1-yl)methyl)-4- (difluoromethoxy)-1-methyl- 1H-benzo[d]imidazole-6- carboxylic acid | 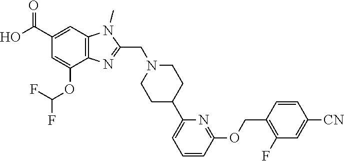 [SMILES: Cn1c(CN2CCC(c3cccc(OCc4ccc(C#N)cc4F)n3)CC2)nc2c(OC(F)F)cc(C(=O)O)cc21] |
| 128 | 4-acetoxy-2-((4-(6-((4- cyano-2- fluorobenzyl)oxy)pyridin-2- yl)piperidin-1-yl)methyl)-1- methyl-1H- benzo[d]imidazole-6- carboxylic acid | 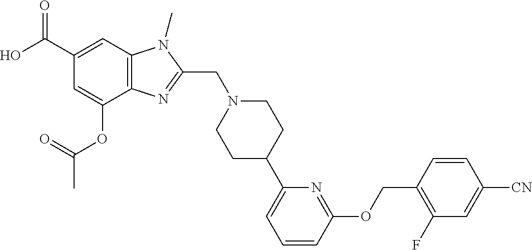 [SMILES: CC(=O)Oc1cc(C(=O)O)cc2c1nc(CN1CCC(c3cccc(OCc4ccc(C#N)cc4F)n3)CC1)n2C] |
| 129 | (S)-2-((4-(6-((4-cyano-2- fluorobenzyl)oxy)pyridin-2- yl)piperidin-1-yl)methyl)-4- (methylsulfonamido)-1- (oxetan-2-ylmethyl)-1H- benzo[d]imidazole-6- carboxylic acid | 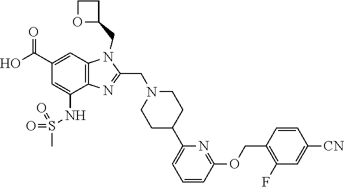 [SMILES: CS(=O)(=O)Nc1cc(C(=O)O)cc2c1nc(CN1CCC(c3cccc(OCc4ccc(C#N)cc4F)n3)CC1)n2C[C@@H]1CCO1] |
| 130 | 2-((4-(6-((4-cyano-2- fluorophenoxy)methyl) pyridin-2-yl)piperidin-1- yl)methyl)-4- (difluoromethoxy)-1-methyl- 1H-benzo[d]imidazole-6- carboxylic acid | 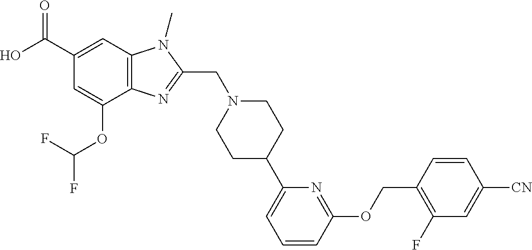 [SMILES: Cn1c(CN2CCC(c3cccc(OCc4ccc(C#N)cc4F)n3)CC2)nc2c(OC(F)F)cc(C(=O)O)cc21] |
| 131 | 2-(((1s,4R)-4-(6-((4-cyano- 2-fluorobenzyl)oxy)pyridin- 2-yl)cyclohexyl) (methyl)amino)-4- (difluoromethoxy)-1- (((S)-oxetan-2-yl)methyl)- 1H-benzo[d]imidazole-6- carboxylic acid | 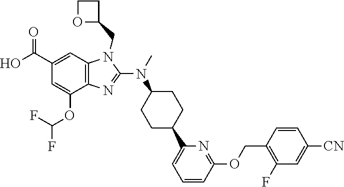 [SMILES: CN(c1nc2c(OC(F)F)cc(C(=O)O)cc2n1C[C@@H]1CCO1)[C@H]1CC[C@@H](c2cccc(OCc3ccc(C#N)cc3F)n2)CC1] |
| 132 | 2-(((1r,4S)-4-(6-((4-cyano-2- fluorobenzyl)oxy)pyridin-2- yl)cyclohexyl) (methyl)amino)-4- (difluoromethoxy)-1- (((S)-oxetan-2-yl)methyl)- 1H-benzo[d]imidazole-6- carboxylic acid | 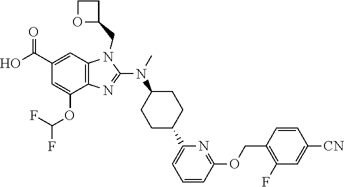 [SMILES: CN(c1nc2c(OC(F)F)cc(C(=O)O)cc2n1C[C@@H]1CCO1)[C@H]1CC[C@H](c2cccc(OCc3ccc(C#N)cc3F)n2)CC1] |
| 133 | 2-(((1r,4r)-4-(6-((4-cyano-2- fluorobenzyl)oxy)pyridin-2- yl)cyclohexyl)(methyl) amino)-4- (difluoromethoxy)- 1-methyl-1H- benzo[d]imidazole-6- carboxylic acid | 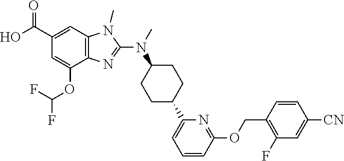 [SMILES: Cn1c(N(C)[C@H]2CC[C@H](c3cccc(OCc4ccc(C#N)cc4F)n3)CC2)nc2c(OC(F)F)cc(C(=O)O)cc21] |
| 134 | (S)-4-amino-2-((4-(6-((4- cyano-2- fluorobenzyl)oxy)pyridin-2- yl)piperidin-1-yl)methyl)-1- (oxetan-2-ylmethyl)-1H- imidazo[4,5-c]pyridine-6- carboxylic acid | 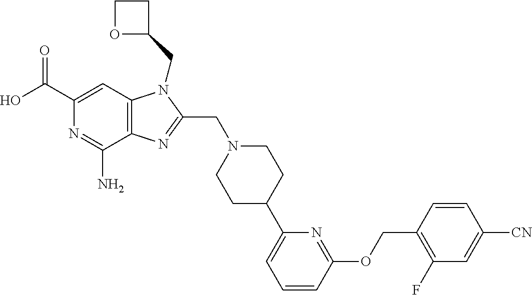 [SMILES: N#Cc1ccc(COc2cccc(C3CCN(Cc4nc5c(N)nc(C(=O)O)cc5n4C[C@@H]4CCO4)CC3)n2)c(F)c1] |
| 135 | 4-(tert-butoxy)-2-((4-(6-((4- cyano-2- fluorobenzyl)oxy)pyridin-2- yl)piperidin-1-yl)methyl)-1- methyl-1H- benzo[d]imidazole-6- carboxylic acid | 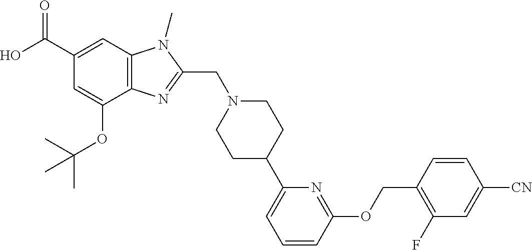 [SMILES: Cn1c(CN2CCC(c3cccc(OCc4ccc(C#N)cc4F)n3)CC2)nc2c(OC(C)(C)C)cc(C(=O)O)cc21] |
| 136 | 2-((4-(6-((4-chloro-2- fluorobenzyl)oxy)pyridin-2- yl)piperidin-1-yl)methyl)-4- (difluoromethoxy)-1-methyl- 1H-benzo[d]imidazole-6- carboxylic acid | 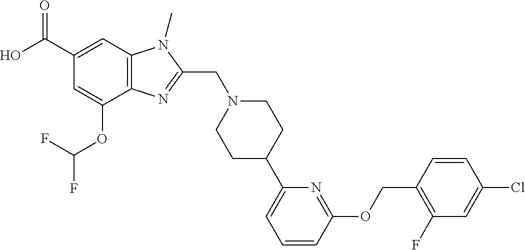 [SMILES: Cn1c(CN2CCC(c3cccc(OCc4ccc(Cl)cc4F)n3)CC2)nc2c(OC(F)F)cc(C(=O)O)cc21] |
| 137 | 2-((4-(6-((4-cyano-2- fluorobenzyl)oxy)-5- fluoropyridin-2-yl)piperidin- 1-yl)methyl)-4- (difluoromethoxy)-1-methyl- 1H-benzo[d]imidazole-6- carboxylic acid | 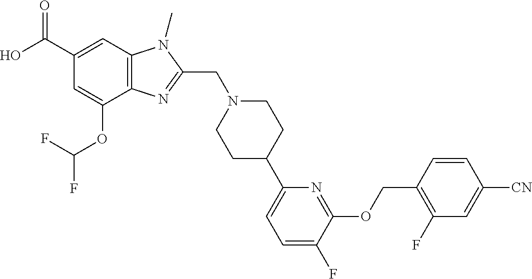 [SMILES: Cn1c(CN2CCC(c3ccc(F)c(OCc4ccc(C#N)cc4F)n3)CC2)nc2c(OC(F)F)cc(C(=O)O)cc21] |
| 138 | 2-((4-(4-chloro-2-((4-cyano- 2- fluorobenzyl)oxy)phenyl) piperidin-1-yl)methyl)-4- (difluoromethoxy)-1-methyl- 1H-benzo[d]imidazole-6- carboxylic acid | 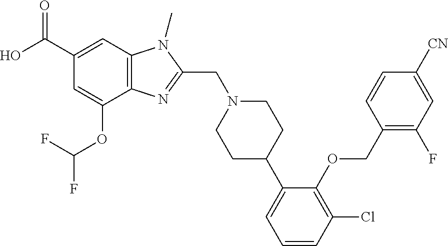 [SMILES: Cn1c(CN2CCC(c3cccc(Cl)c3OCc3ccc(C#N)cc3F)CC2)nc2c(OC(F)F)cc(C(=O)O)cc21] |
| 139 | 4-(difluoromethoxy)-2-((4- (4-fluoro-2-((2-fluoro-4- methylbenzyl)oxy)phenyl) piperidin-1-yl)methyl)-1- methyl-1H- benzo[d]imidazole-6- carboxylic acid | 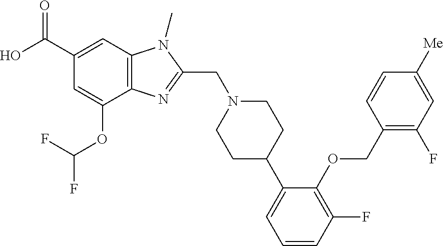 [SMILES: Cc1ccc(COc2c(F)cccc2C2CCN(Cc3nc4c(OC(F)F)cc(C(=O)O)cc4n3C)CC2)c(F)c1] |
| 140 | 2-((4-(2-((4-chloro-2- fluorobenzyl)oxy)-4- fluorophenyl)piperidin-1- yl)methyl)-4- (difluoromethoxy)-1-methyl- 1H-benzo[d]imidazole-6- carboxylic acid | 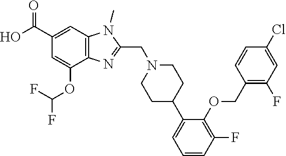 [SMILES: Cn1c(CN2CCC(c3cccc(F)c3OCc3ccc(Cl)cc3F)CC2)nc2c(OC(F)F)cc(C(=O)O)cc21] |
| 141 | 4-(difluoromethoxy)-2-((4- (6-((2-fluoro-4-(oxetan-3- yl)benzyl)oxy)pyridin-2- yl)piperidin-1-yl)methyl)-1- methyl-1H- benzo[d]imidazole-6- carboxylic acid | 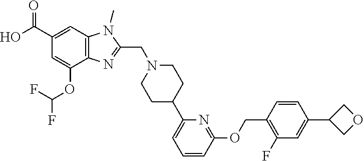 [SMILES: Cn1c(CN2CCC(c3cccc(OCc4ccc(C5COC5)cc4F)n3)CC2)nc2c(OC(F)F)cc(C(=O)O)cc21] |
| 142 | 2-(((1s,4s)-4-(6-((4-cyano-2- fluorobenzyl)oxy)pyridin-2- yl)cyclohexyl) (methyl)amino)- 4-(difluoromethoxy)-1- methyl-1H- benzo[d]imidazole-6- carboxylic acid | 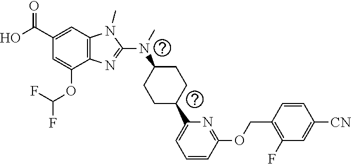 [SMILES: Cn1c(N(C)[C@H]2CC[C@@H](c3cccc(OCc4ccc(C#N)cc4F)n3)CC2)nc2c(OC(F)F)cc(C(=O)O)cc21] |
| 143 | (S)-2-((4-(6-((4-cyano-2- fluorobenzyl)oxy)pyridin-2- yl)piperidin-1-yl)methyl)-4- hydroxy-1-(oxetan-2- ylmethyl)-1H-imidazo[4,5- c]pyridine-6-carboxylic acid | 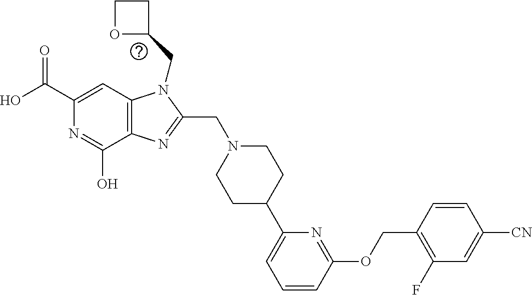 [SMILES: N#Cc1ccc(COc2cccc(C3CCN(Cc4nc5c(O)nc(C(=O)O)cc5n4C[C@@H]4CCO4)CC3)n2)c(F)c1] |
| 144 | 2-((4-(3-((4-cyano-2- fluorobenzyl)oxy)-2- methoxyphenyl)piperidin-1- yl)methyl)-4- (difluoromethoxy)-1-methyl- 1H-benzo[d]imidazole-6- carboxylic acid | 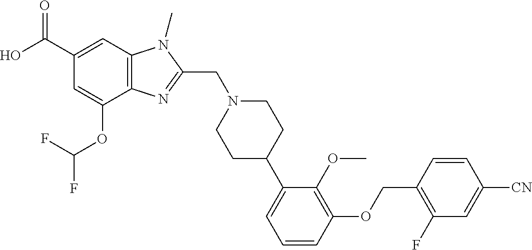 [SMILES: COc1c(OCc2ccc(C#N)cc2F)cccc1C1CCN(Cc2nc3c(OC(F)F)cc(C(=O)O)cc3n2C)CC1] |
| 145 | 2-((4-(2-((4-cyano-2- fluorobenzyl)oxy)pyrimidin- 4-yl)piperidin-1-yl)methyl)- 4-(difluoromethoxy)-1- methyl-1H- benzo[d]imidazole-6- carboxylic acid | 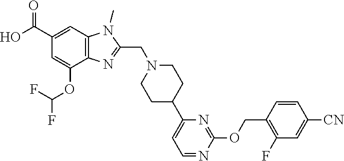 [SMILES: Cn1c(CN2CCC(c3ccnc(OCc4ccc(C#N)cc4F)n3)CC2)nc2c(OC(F)F)cc(C(=O)O)cc21] |
| 146 | 2-((4-(2-((4-cyano-2- fluorobenzyl)oxy)-6- fluorophenyl)piperidin-1- yl)methyl)-4- (difluoromethoxy)-1-methyl- 1H-benzo[d]imidazole-6- carboxylic acid | 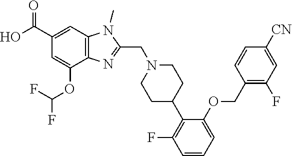 [SMILES: Cn1c(CN2CCC(c3c(F)cccc3OCc3ccc(C#N)cc3F)CC2)nc2c(OC(F)F)cc(C(=O)O)cc21] |
| 147 | 2-((4-(6-((4-cyano-2- fluorobenzyl)oxy)pyridin-2- yl)piperidin-1-yl)methyl)-1- (difluoromethyl)-4-methoxy- 1H-benzo[d]imidazole-6- carboxylic acid | 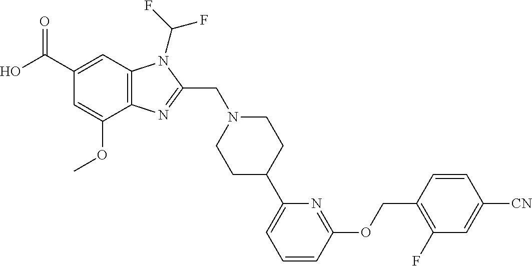 [SMILES: COc1cc(C(=O)O)cc2c1nc(CN1CCC(c3cccc(OCc4ccc(C#N)cc4F)n3)CC1)n2C(F)F] |
| 148 | 2-((4-(3-((4-cyano-2- fluorobenzyl)oxy)phenyl) piperidin-1-yl)methyl)-4- (difluoromethoxy)-1-methyl- 1H-benzo[d]imidazole-6- carboxylic acid | 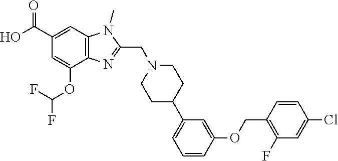 [SMILES: Cn1c(CN2CCC(c3cccc(OCc4ccc(Cl)cc4F)c3)CC2)nc2c(OC(F)F)cc(C(=O)O)cc21] |
| 149 | 2-((4-((6-((4-cyano-2- fluorophenoxy)methyl) pyridin-2-yl)oxy)piperidin- 1-yl)methyl)-4- (difluoromethoxy)-1-methyl- 1H-benzo[d]imidazole-6- carboxylic acid | 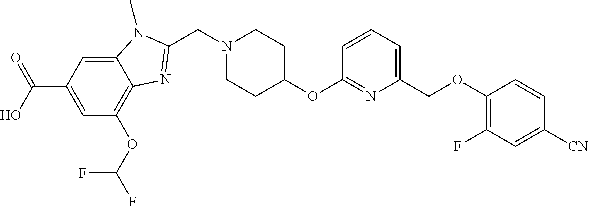 [SMILES: Cn1c(CN2CCC(Oc3cccc(COc4ccc(C#N)cc4F)n3)CC2)nc2c(OC(F)F)cc(C(=O)O)cc21] |
| 150 | 2-((4-(6-((4-cyano-2- fluorobenzyl)oxy)pyridin-2- yl)piperazin-1-yl)methyl)-4- (difluoromethoxy)-1-methyl- 1H-benzo[d]imidazole-6- carboxylic acid | 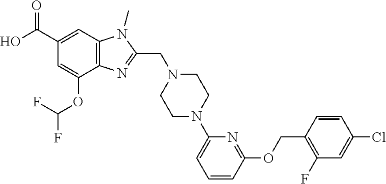 [SMILES: Cn1c(CN2CCN(c3cccc(OCc4ccc(Cl)cc4F)n3)CC2)nc2c(OC(F)F)cc(C(=O)O)cc21] |
| 151 | 2-((4-(6-(6-cyano-8-fluoro- 3,4-dihydroisoquinolin- 2(1H)-yl)pyridin-2- yl)piperidin-1-yl)methyl)-4- (difluoromethoxy)-1-methyl- 1H-benzo[d]imidazole-6- carboxylic acid | 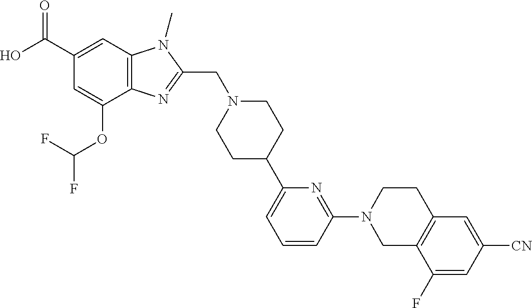 [SMILES: Cn1c(CN2CCC(c3cccc(N4CCc5cc(C#N)cc(F)c5C4)n3)CC2)nc2c(OC(F)F)cc(C(=O)O)cc21] |
| 152 | 2-((4-(3-((4-chloro-2- fluorobenzyl)oxy)-4- fluorophenyl)-3,6- dihydropyridin-1(2H)- yl)methyl)-4- (difluoromethoxy)-1-methyl- 1H-benzo[d]imidazole-6- carboxylic acid | 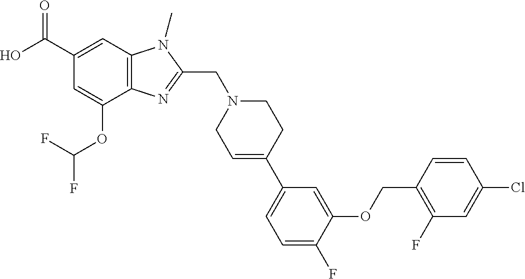 [SMILES: Cn1c(CN2CC=C(c3ccc(F)c(OCc4ccc(Cl)cc4F)c3)CC2)nc2c(OC(F)F)cc(C(=O)O)cc21] |
| 153 | 2-((4-(6-((4-chloro-2,6- difluorobenzyl)oxy)pyridin- 2-yl)piperidin-1-yl)methyl)- 4-(difluoromethoxy)-1- methyl-1H- benzo[d]imidazole-6- carboxylic acid | 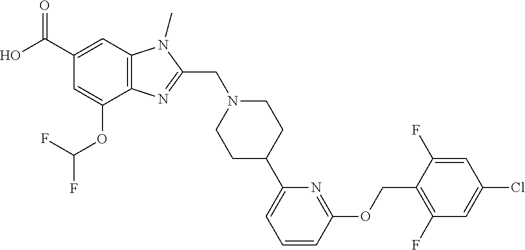 [SMILES: Cn1c(CN2CCC(c3cccc(OCc4c(F)cc(Cl)cc4F)n3)CC2)nc2c(OC(F)F)cc(C(=O)O)cc21] |
| 154 | 4-(difluoromethoxy)-2-((4- (6-((2-methoxy-4- (trifluoromethyl)benzyl)oxy) pyridin-2-yl)piperidin-1- yl)methyl)-1-methyl-1H- benzo[d]imidazole-6- carboxylic acid | 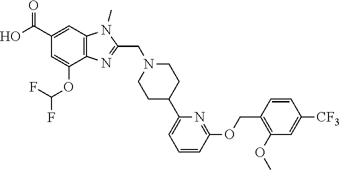 [SMILES: COc1cc(C(F)(F)F)ccc1COc1cccc(C2CCN(Cc3nc4c(OC(F)F)cc(C(=O)O)cc4n3C)CC2)n1] |
| 155 | 2-((6-((4-chloro-2- fluorobenzyl)oxy)-3′,6′- dihydro-[2,4′-bipyridin]- 1′(2′H)-yl)methyl)-4- (difluoromethoxy)-1-methyl- 1H-benzo[d]imidazole-6- carboxylic acid | 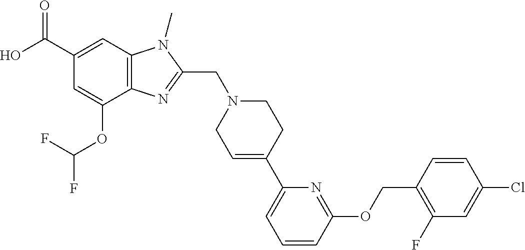 [SMILES: Cn1c(CN2CC=C(c3cccc(OCc4ccc(Cl)cc4F)n3)CC2)nc2c(OC(F)F)cc(C(=O)O)cc21] |
| 156 | 2-((4-(6-((4-cyano-2- methoxybenzyl)oxy)pyridin- 2-yl)piperidin-1-yl)methyl)- 4-(difluoromethoxy)-1- methyl-1H- benzo[d]imidazole-6- carboxylic acid | 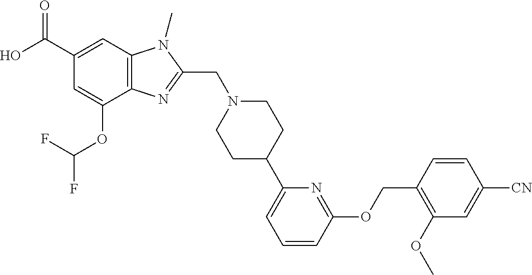 [SMILES: COc1cc(C#N)ccc1COc1cccc(C2CCN(Cc3nc4c(OC(F)F)cc(C(=O)O)cc4n3C)CC2)n1] |
| 157 | 2-((4-((6-(4-cyano-2- fluorobenzyl)pyridin-2- yl)oxy)piperidin-1- yl)methyl)-4- (difluoromethoxy)-1-methyl- 1H-benzo[d]imidazole-6- carboxylic acid | 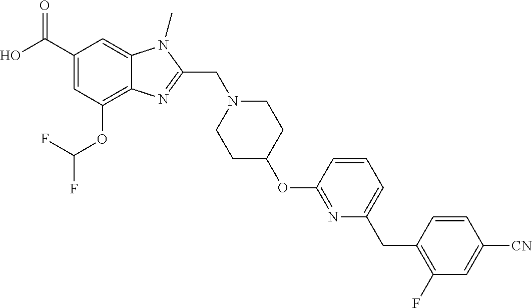 [SMILES: Cn1c(CN2CCC(Oc3cccc(Cc4ccc(C#N)cc4F)n3)CC2)nc2c(OC(F)F)cc(C(=O)O)cc21] |
| 158 | 2-((4-(3-((4-cyano-2- fluorobenzyl)oxy)pyridin-2- yl)piperidin-1-yl)methyl)-4- (difluoromethoxy)-1-methyl- 1H-benzo[d]imidazole-6- carboxylic acid | 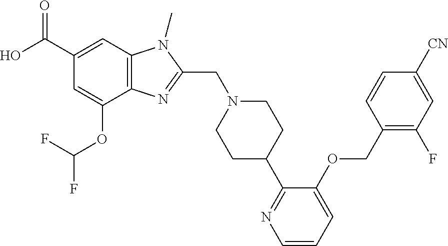 [SMILES: Cn1c(CN2CCC(c3ncccc3OCc3ccc(C#N)cc3F)CC2)nc2c(OC(F)F)cc(C(=O)O)cc21] |
| 159 | 2-((4-(3-((4-chloro-2- fluorobenzyl)oxy)phenyl)- 3,6-dihydropyridin-1(2H)- yl)methyl)-4- (difluoromethoxy)-1-methyl- 1H-benzo[d]imidazole-6- carboxylic acid | 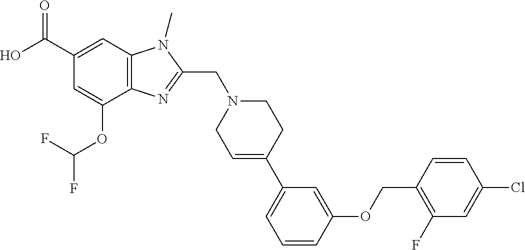 [SMILES: Cn1c(CN2CC=C(c3cccc(OCc4ccc(Cl)cc4F)c3)CC2)nc2c(OC(F)F)cc(C(=O)O)cc21] |
| 160 | 2-((4-(6-((4-cyano-2- fluorobenzyl)oxy)pyridin-2- yl)piperidin-1-yl)methyl)-4- ((1-fluoro-2-methylpropan-2- yl)oxy)-1-methyl-1H- benzo[d]imidazole-6- carboxylic acid | 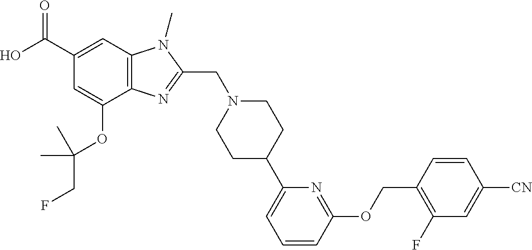 [SMILES: Cn1c(CN2CCC(c3cccc(OCc4ccc(C#N)cc4F)n3)CC2)nc2c(OC(C)(C)CF)cc(C(=O)O)cc21] |
| 161 | 2-((6-((4-chloro-2- fluorobenzyl)oxy)-5′-fluoro- [2,3′-bipyridin]-6′- yl)methyl)-4- (difluoromethoxy)-1-methyl- 1H-benzo[d]imidazole-6- carboxylic acid | 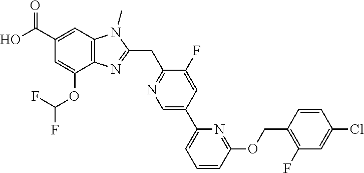 [SMILES: Cn1c(Cc2ncc(-c3cccc(OCc4ccc(Cl)cc4F)n3)cc2F)nc2c(OC(F)F)cc(C(=O)O)cc21] |
| 162 | 2-(4-(6-((4-chloro-2- fluorobenzyl)oxy)pyridin-2- yl)-2,6-difluorobenzyl)-4- (difluoromethoxy)-1-methyl- 1H-benzo[d]imidazole-6- carboxylic acid | 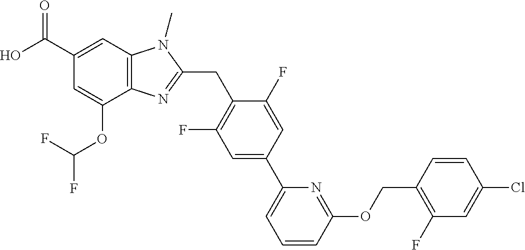 [SMILES: Cn1c(Cc2c(F)cc(-c3cccc(OCc4ccc(Cl)cc4F)n3)cc2F)nc2c(OC(F)F)cc(C(=O)O)cc21] |
| 163 | 2-(4-(6-((4-chloro-2- fluorobenzyl)oxy)pyridin-2- yl)-2,3,6-trifluorobenzyl)-4- (difluoromethoxy)-1-methyl- 1H-benzo[d]imidazole-6- carboxylic acid | 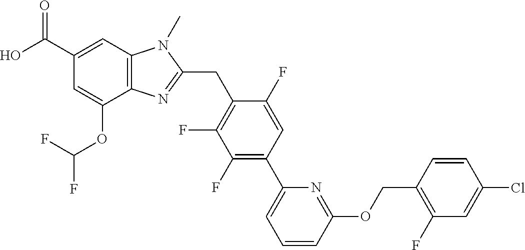 [SMILES: Cn1c(Cc2c(F)cc(-c3cccc(OCc4ccc(Cl)cc4F)n3)c(F)c2F)nc2c(OC(F)F)cc(C(=O)O)cc21] |
| 164 | 2-(4-(6-((4-chloro-2- fluorobenzyl)oxy)pyridin-2- yl)-3-fluorobenzyl)-4- (difluoromethoxy)-1-methyl- 1H-benzo[d]imidazole-6- carboxylic acid | 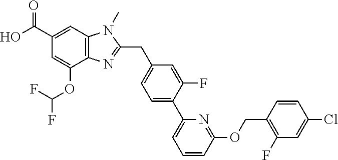 [SMILES: Cn1c(Cc2ccc(-c3cccc(OCc4ccc(Cl)cc4F)n3)c(F)c2)nc2c(OC(F)F)cc(C(=O)O)cc21] |
| 165 | 2-(4-(6-((4-chloro-2- fluorobenzyl)oxy)pyridin-2- yl)-2-fluoro-5- methylbenzyl)-4- (difluoromethoxy)-1-methyl- 1H-benzo[d]imidazole-6- carboxylic acid | 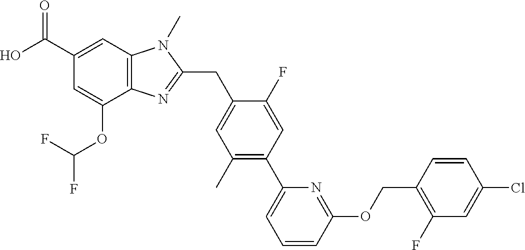 [SMILES: Cc1cc(Cc2nc3c(OC(F)F)cc(C(=O)O)cc3n2C)c(F)cc1-c1cccc(OCc2ccc(Cl)cc2F)n1] |
| 166 | 2-(4-(6-((4-chloro-2- fluorobenzyl)oxy)pyridin-2- yl)benzyl)-4- (difluoromethoxy)-1-methyl- 1H-benzo[d]imidazole-6- carboxylic acid | 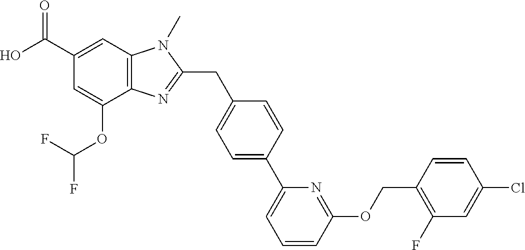 [SMILES: Cn1c(Cc2ccc(-c3cccc(OCc4ccc(Cl)cc4F)n3)cc2)nc2c(OC(F)F)cc(C(=O)O)cc21] |
| 167 | 2-((4-(5-((4-chloro-2- fluorobenzyl)oxy)-2- fluorophenyl)piperidin-1- yl)methyl)-4- (difluoromethoxy)-1-methyl- 1H-benzo[d]imidazole-6- carboxylic acid | 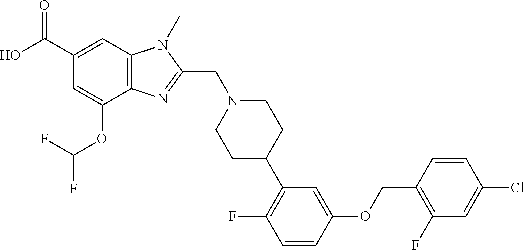 [SMILES: Cn1c(CN2CCC(c3cc(OCc4ccc(Cl)cc4F)ccc3F)CC2)nc2c(OC(F)F)cc(C(=O)O)cc21] |
| 168 | 2-((4-(4-((4-chloro-2- fluorobenzyl)oxy)pyrimidin- 2-yl)piperidin-1-yl)methyl)- 4-(difluoromethoxy)-1- methyl-1H- benzo[d]imidazole-6- carboxylic acid | 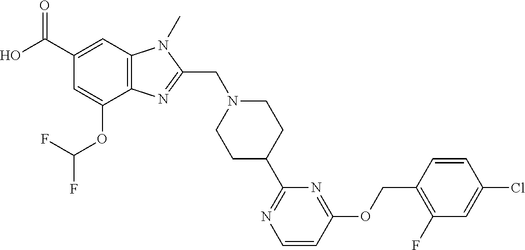 [SMILES: Cn1c(CN2CCC(c3nccc(OCc4ccc(Cl)cc4F)n3)CC2)nc2c(OC(F)F)cc(C(=O)O)cc21] |
| 169 | 2-((4-(6-((4-chloro-2- fluorobenzyl)oxy)pyridin-2- yl)piperidin-1-yl)methyl)-4- (2,2-difluoroethoxy)-1- methyl-1H- benzo[d]imidazole-6- carboxylic acid | 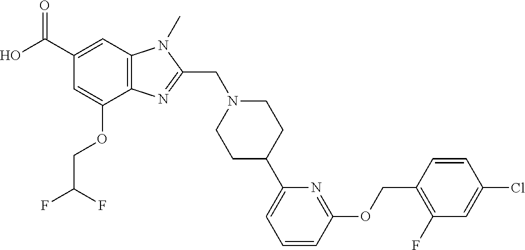 [SMILES: Cn1c(CN2CCC(c3cccc(OCc4ccc(Cl)cc4F)n3)CC2)nc2c(OCC(F)F)cc(C(=O)O)cc21] |
| 170 | 2-((4-(6-((4-chloro-2- fluorobenzyl)oxy)pyridin-2- yl)piperidin-1-yl)methyl)-4- (2-fluoroethoxy)-1-methyl- 1H-benzo[d]imidazole-6- carboxylic acid | 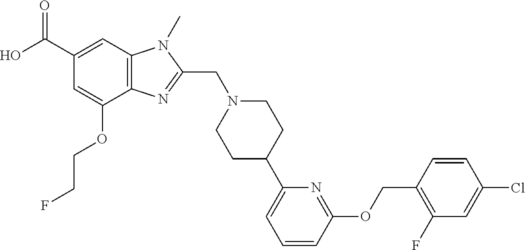 [SMILES: Cn1c(CN2CCC(c3cccc(OCc4ccc(Cl)cc4F)n3)CC2)nc2c(OCCF)cc(C(=O)O)cc21] |
| 171 | 2-((4-(3-((4-chloro-2- fluorobenzyl)oxy)-2- methoxyphenyl)piperidin-1- yl)methyl)-4- (difluoromethoxy)-1-methyl- 1H-benzo[d]imidazole-6- carboxylic acid | 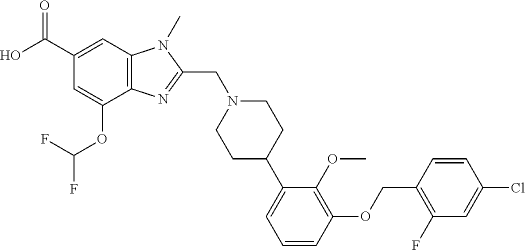 [SMILES: COc1c(OCc2ccc(Cl)cc2F)cccc1C1CCN(Cc2nc3c(OC(F)F)cc(C(=O)O)cc3n2C)CC1] |
| 172 | (S)-2-((4-(6-((4-cyano-2- fluorobenzyl)oxy)pyridin-2- yl)piperidin-1-yl)methyl)-7- hydroxy-1-(oxetan-2- ylmethyl)-1H- benzo[d]imidazole-6- carboxylic acid | 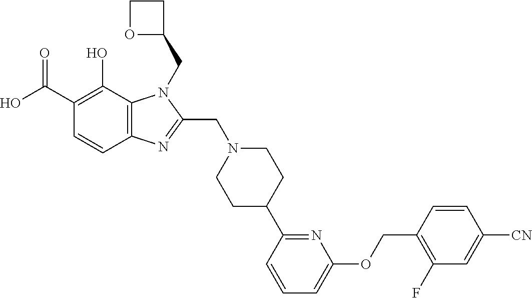 [SMILES: N#Cc1ccc(COc2cccc(C3CCN(Cc4nc5ccc(C(=O)O)c(O)c5n4C[C@@H]4CCO4)CC3)n2)c(F)c1] |
| 173 | (R)-2-((4-(2-(1-(4-cyano-2- fluorophenyl)ethoxy)phenyl) piperidin-1-yl)methyl)-4- (difluoromethoxy)-1-methyl- 1H-benzo[d]imidazole-6- carboxylic acid | 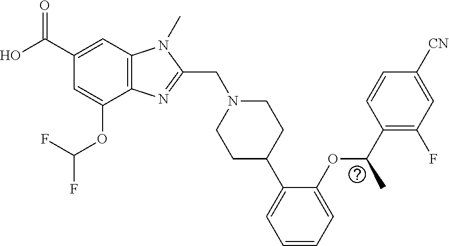 [SMILES: C[C@@H](Oc1ccccc1C1CCN(Cc2nc3c(OC(F)F)cc(C(=O)O)cc3n2C)CC1)c1ccc(C#N)cc1F] |
| 174 | (S)-2-((4-(2-(1-(4-cyano-2- fluorophenyl)ethoxy)phenyl) piperidin-1-yl)methyl)-4- (difluoromethoxy)-1-methyl- 1H-benzo[d]imidazole-6- carboxylic acid | 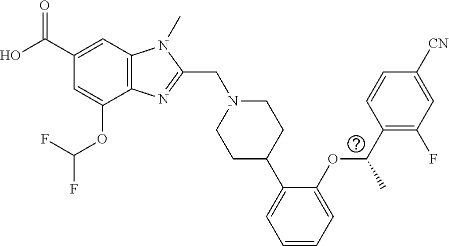 [SMILES: C[C@H](Oc1ccccc1C1CCN(Cc2nc3c(OC(F)F)cc(C(=O)O)cc3n2C)CC1)c1ccc(C#N)cc1F] |
| 175 | (R)-2-((4-(2-(4-chloro-2- fluorophenyl)-2H-chromen- 8-yl)piperidin-1-yl)methyl)- 4-(difluoromethoxy)-1- methyl-1H- benzo[d]imidazole-6- carboxylic acid | 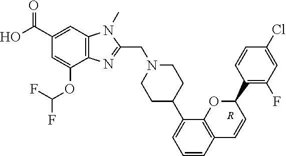 |
| 176 | (S)-2-((4-(2-(4-chloro-2- fluorophenyl)-2H-chromen- 8-yl)piperidin-1-yl)methyl)- 4-(difluoromethoxy)-1- methyl-1H- benzo[d]imidazole-6- carboxylic acid | 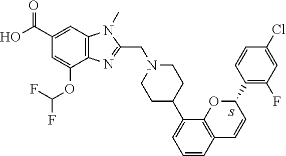 |
| 177 | 4-(difluoromethoxy)-2-((4- (6-((2-fluoro-4- (tetrahydrofuran-2- yl)benzyl)oxy)pyridin-2- yl)piperidin-1-yl)methyl)-1- methyl-1H- benzo[d]imidazole-6- carboxylic acid | 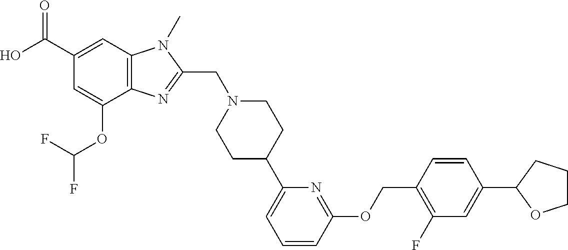 [SMILES: Cn1c(CN2CCC(c3cccc(OCc4ccc(C5CCCO5)cc4F)n3)CC2)nc2c(OC(F)F)cc(C(=O)O)cc21] |
| 178 | 2-(((1r,4r)-4-(6-((4-cyano-2- fluorobenzyl)oxy)pyridin-2- yl)cyclohexyl) (methyl)amino)- 4-methoxy-1-methyl-1H- benzo[d]imidazole-6- carboxylic acid | 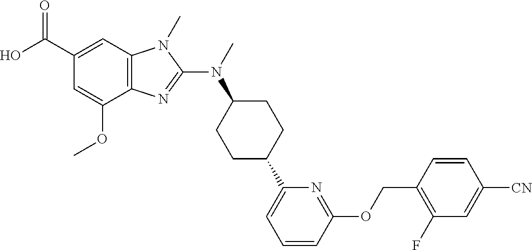 [SMILES: COc1cc(C(=O)O)cc2c1nc(N(C)[C@H]1CC[C@H](c3cccc(OCc4ccc(C#N)cc4F)n3)CC1)n2C] |
| 179 | 2-((4-(2-chloro-3-((4-cyano- 2- fluorobenzyl)oxy)phenyl) piperidin-1-yl)methyl)-4- (difluoromethoxy)-1-methyl- 1H-benzo[d]imidazole-6- carboxylic acid | 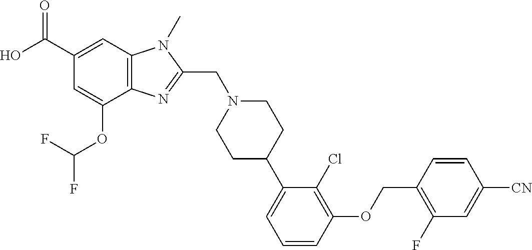 [SMILES: Cn1c(CN2CCC(c3cccc(OCc4ccc(C#N)cc4F)c3Cl)CC2)nc2c(OC(F)F)cc(C(=O)O)cc21] |
| 180 | 2-((4-(6-((4-cyano-2- fluorobenzyl)oxy)pyridin-2- yl)piperidin-1-yl)methyl)-4- (1,1-difluoroethoxy)-1- methyl-1H- benzo[d]imidazole-6- carboxylic acid | 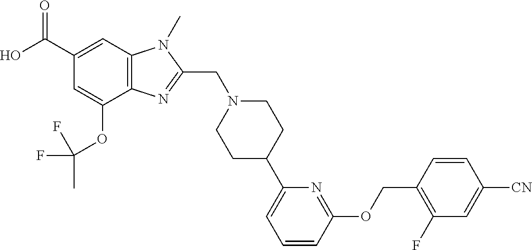 [SMILES: Cn1c(CN2CCC(c3cccc(OCc4ccc(C#N)cc4F)n3)CC2)nc2c(OC(C)(F)F)cc(C(=O)O)cc21] |
| 181 | 2-((4-(5-((4-cyano-2- fluorobenzyl)oxy)-2- fluorophenyl)piperidin-1- yl)methyl)-4- (difluoromethoxy)-1-methyl- 1H-benzo[d]imidazole-6- carboxylic acid | 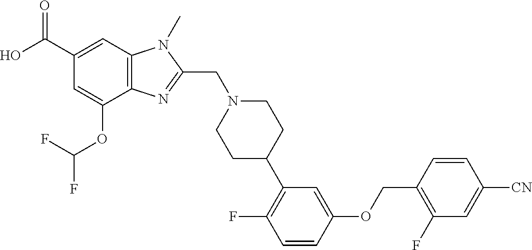 [SMILES: Cn1c(CN2CCC(c3cc(OCc4ccc(C#N)cc4F)ccc3F)CC2)nc2c(OC(F)F)cc(C(=O)O)cc21] |
| 182 | 2-((4-(2-((4-cyano-2- fluorobenzyl)oxy)-3- fluorophenyl)piperidin-1- yl)methyl)-4- (difluoromethoxy)-1-methyl- 1H-benzo[d]imidazole-6- carboxylic acid | 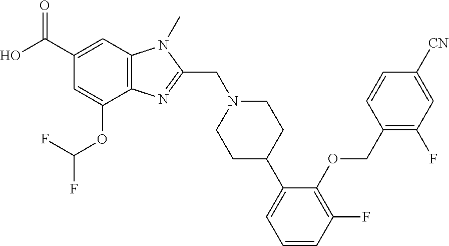 [SMILES: Cn1c(CN2CCC(c3cccc(F)c3OCc3ccc(C#N)cc3F)CC2)nc2c(OC(F)F)cc(C(=O)O)cc21] |
| 183 | 2-((4-(6-((4-cyano-2- fluorobenzyl)oxy)pyridin-2- yl)piperidin-1-yl)methyl)-4- (2-fluoroethoxy)-1-methyl- 1H-benzo[d]imidazole-6- carboxylic acid | 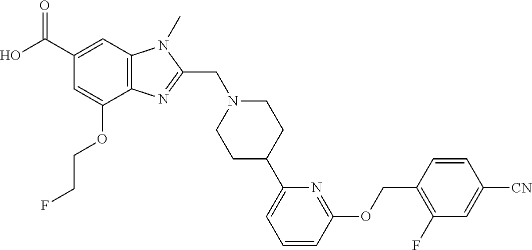 [SMILES: Cn1c(CN2CCC(c3cccc(OCc4ccc(C#N)cc4F)n3)CC2)nc2c(OCCF)cc(C(=O)O)cc21] |
| 184 | 2-((4-(4-((4-cyano-2- fluorobenzyl)oxy)pyrimidin- 2-yl)piperidin-1-yl)methyl)- 4-(difluoromethoxy)-1- methyl-1H- benzo[d]imidazole-6- carboxylic acid | 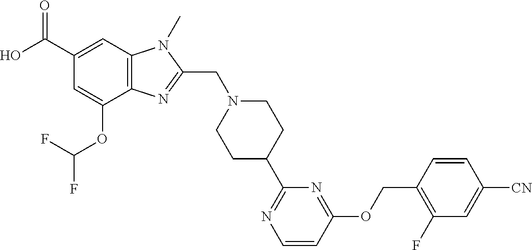 [SMILES: Cn1c(CN2CCC(c3nccc(OCc4ccc(C#N)cc4F)n3)CC2)nc2c(OC(F)F)cc(C(=O)O)cc21] |
| 185 | 2-((4-(6-((4-carbamoyl-2- fluorobenzyl)oxy)-5- fluoropyridin-2-yl)piperidin- 1-yl)methyl)-4- (difluoromethoxy)-1-methyl- 1H-benzo[d]imidazole-6- carboxylic acid | 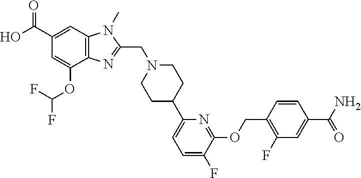 [SMILES: Cn1c(CN2CCC(c3ccc(F)c(OCc4ccc(C(N)=O)cc4F)n3)CC2)nc2c(OC(F)F)cc(C(=O)O)cc21] |
| 186 | 2-((4-(6-((4-Chloro-2- fluorobenzyl)oxy)pyridin-2- yl)piperidin-1-yl)methyl)-4- ((2-fluoroethyl)amino)-1- methyl-1H- benzo[d]imidazole-6- carboxylic acid | 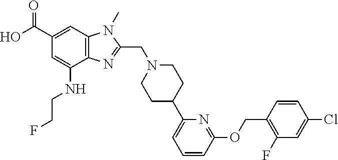 [SMILES: Cn1c(CN2CCC(c3cccc(OCc4ccc(Cl)cc4F)n3)CC2)nc2c(NCCF)cc(C(=O)O)cc21] |
| 187 | 2-((4-(6-((4-Chloro-2- fluorobenzyl)oxy)pyridin-2- yl)piperidin-1-yl)methyl)-4- methoxy-1-methyl-1H- benzo[d]imidazole-5- carboxylic acid | 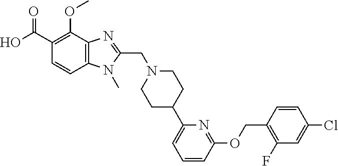 [SMILES: COc1c(C(=O)O)ccc2c1nc(CN1CCC(c3cccc(OCc4ccc(Cl)cc4F)n3)CC1)n2C] |
| 188 | 2-((4-(6-((4-Chloro-2- fluorobenzyl)oxy)pyridin-2- yl)piperidin-1-yl)methyl)-4- ((1- fluorocyclopropyl)methoxy)- 1-methyl-1H- benzo[d]imidazole-6- carboxylic acid | 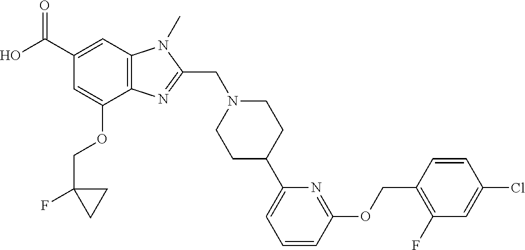 [SMILES: Cn1c(CN2CCC(c3cccc(OCc4ccc(Cl)cc4F)n3)CC2)nc2c(OCC3(F)CC3)cc(C(=O)O)cc21] |
| 189 | 2-((4-(6-((4-chloro-2- fluorobenzyl)oxy)pyridin-2- yl)piperidin-1-yl)methyl)-4- cyano-1-methyl-1H- benzo[d]imidazole-6- carboxylic acid | 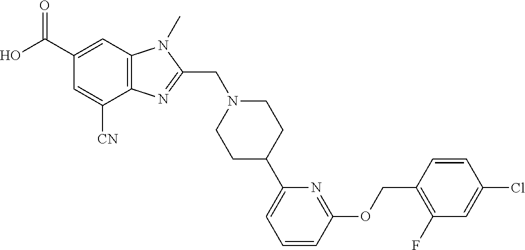 [SMILES: Cn1c(CN2CCC(c3cccc(OCc4ccc(Cl)cc4F)n3)CC2)nc2c(C#N)cc(C(=O)O)cc21] |
| 190 | 2-((4-(6-((4-Chloro-2- fluorobenzyl)oxy)pyridin-2- yl)piperidin-1-yl)methyl)-4- (difluoromethoxy)-7-fluoro- 1-methyl-1H- benzo[d]imidazole-6- carboxylic acid | 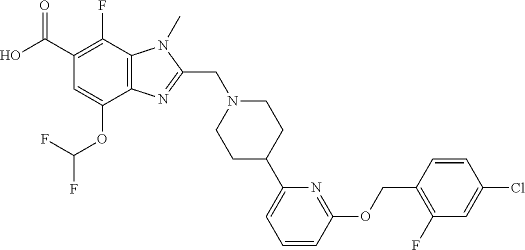 [SMILES: Cn1c(CN2CCC(c3cccc(OCc4ccc(Cl)cc4F)n3)CC2)nc2c(OC(F)F)cc(C(=O)O)c(F)c21] |
| 191 | (S)-2-((4-(6-((4-chloro-2- fluorobenzyl)oxy)pyridin-2- yl)piperidin-1-yl)methyl)-4- (difluoromethoxy)-1- (oxetan-2-ylmethyl)-1H- benzo[d]imidazole-5- carboxylic acid | 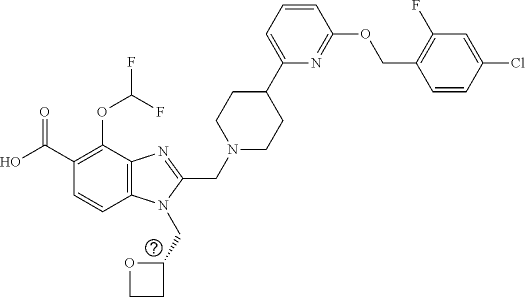 [SMILES: O=C(O)c1ccc2c(nc(CN3CCC(c4cccc(OCc5ccc(Cl)cc5F)n4)CC3)n2C[C@@H]2CCO2)c1OC(F)F] |
| 192 | 2-((4-(6-((4-Chloro-2- fluorobenzyl)oxy)pyridin-2- yl)piperidin-1-yl)methyl)-1- (fluoromethyl)-7-methoxy- 1H-benzo[d]imidazole-5- carboxylic acid | 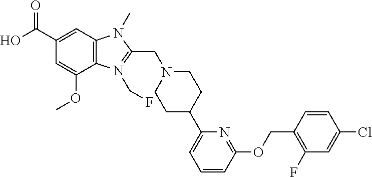 |
| 193 | 2-((4-(4-(4-chloro-2- fluorobenzyl)-3-oxo-3,4- dihydro-2H- benzo[b][1,4]oxazin-6- yl)piperidin-1-yl)methyl)-4- (difluoromethoxy)-1-methyl- 1H-benzo[d]imidazole-6- carboxylic acid | 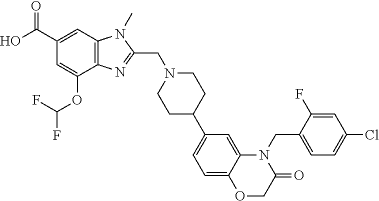 [SMILES: Cn1c(CN2CCC(c3ccc4c(c3)N(Cc3ccc(Cl)cc3F)C(=O)CO4)CC2)nc2c(OC(F)F)cc(C(=O)O)cc21] |
| 194 | 2-((4-(4-Chloro-3-((4-chloro- 2- fluorobenzyl)oxy)phenyl) piperidin-1-yl)methyl)-4- (difluoromethoxy)-1-methyl- 1H-benzo[d]imidazole-6- carboxylic acid | 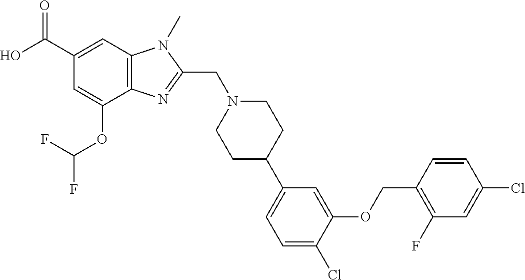 [SMILES: Cn1c(CN2CCC(c3ccc(Cl)c(OCc4ccc(Cl)cc4F)c3)CC2)nc2c(OC(F)F)cc(C(=O)O)cc21] |
| 195 | 2-((4-(6-((4-Chloro-2- fluorophenyl)methoxy- d2)pyridin-2-yl)piperidin-1- yl)methyl)-4- (difluoromethoxy)-1-methyl- 1H-benzo[d]imidazole-6- carboxylic acid | 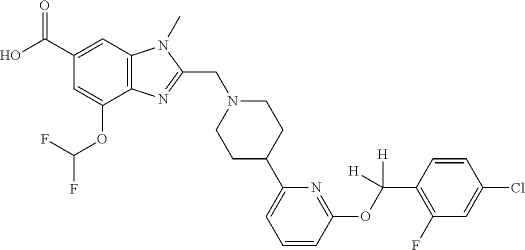 [SMILES: Cn1c(CN2CCC(c3cccc(OCc4ccc(Cl)cc4F)n3)CC2)nc2c(OC(F)F)cc(C(=O)O)cc21] |
| 196 | 2-((4-((6-((4-Chloro-2- fluorophenoxy)methyl)pyridin- 2-yl)oxy)piperidin-1- yl)methyl)-4- (difluoromethoxy)-1-methyl- 1H-benzo[d]imidazole-6- carboxylic acid | 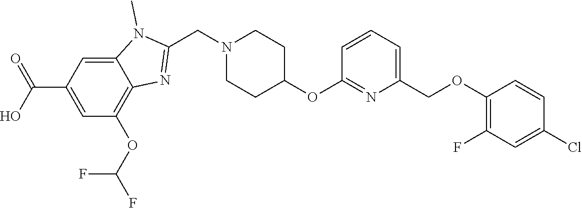 [SMILES: Cn1c(CN2CCC(Oc3cccc(COc4ccc(Cl)cc4F)n3)CC2)nc2c(OC(F)F)cc(C(=O)O)cc21] |
| 197 | 2-(4-(6-((4-Chloro-2- fluorobenzyl)oxy)pyridin-2- yl)-2,5-difluorobenzyl)-4- (difluoromethoxy)-1-methyl- 1H-benzo[d]imidazole-6- carboxylic acid | 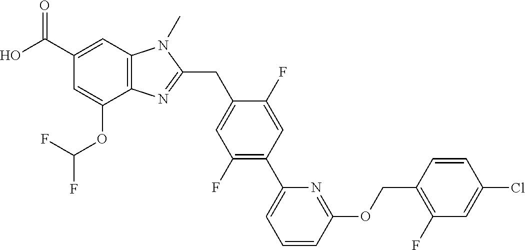 [SMILES: Cn1c(Cc2cc(F)c(-c3cccc(OCc4ccc(Cl)cc4F)n3)cc2F)nc2c(OC(F)F)cc(C(=O)O)cc21] |
| 198 | 2-(4-(6-((4-Chloro-2- fluorobenzyl)oxy)pyridin-2- yl)-2-fluorobenzyl)-4- (difluoromethoxy)-1-methyl- 1H-benzo[d]imidazole-6- carboxylic acid | 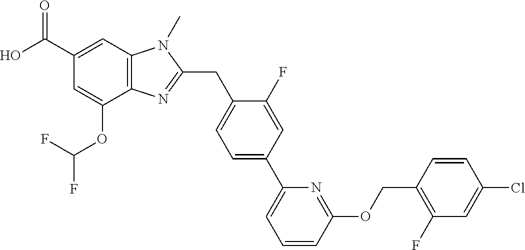 [SMILES: Cn1c(Cc2ccc(-c3cccc(OCc4ccc(Cl)cc4F)n3)cc2F)nc2c(OC(F)F)cc(C(=O)O)cc21] |
| 199 | 2-((4-(6-((4-Chloro-2- fluorobenzyl)oxy)pyridin-2- yl)piperidin-1-yl)methyl)-1- cyclopropyl-4- (difluoromethoxy)-1H- benzo[d]imidazole-6- carboxylic acid | 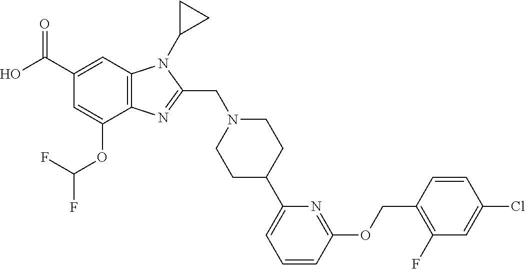 [SMILES: O=C(O)c1cc(OC(F)F)c2nc(CN3CCC(c4cccc(OCc5ccc(Cl)cc5F)n4)CC3)n(C3CC3)c2c1] |
| 200 | 2-((4-(5-((4-Chloro-2- fluorobenzyl)oxy)-1H- pyrazol-1-yl)piperidin-1- yl)methyl)-4- (difluoromethoxy)-1-methyl- 1H-benzo[d]imidazole-6- carboxylic acid | 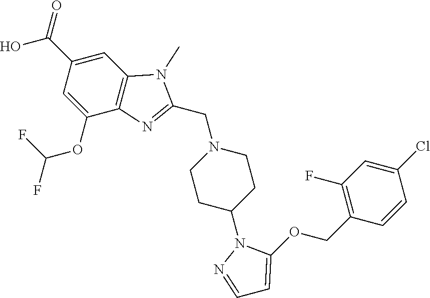 [SMILES: Cn1c(CN2CCC(n3nccc3OCc3ccc(Cl)cc3F)CC2)nc2c(OC(F)F)cc(C(=O)O)cc21] |
| 201 | 4-(Difluoromethoxy)-2-((4- (6-((4-(difluoromethyl)-2- fluorobenzyl)oxy)pyridin-2- yl)piperidin-1-yl)methyl)-1- methyl-1H- benzo[d]imidazole-6- carboxylic acid | 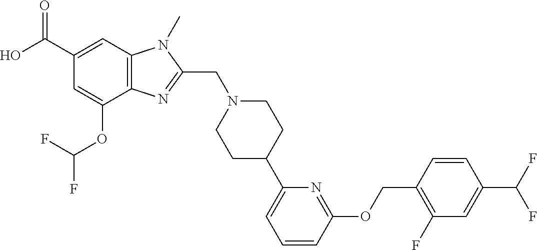 [SMILES: Cn1c(CN2CCC(c3cccc(OCc4ccc(C(F)F)cc4F)n3)CC2)nc2c(OC(F)F)cc(C(=O)O)cc21] |
| 202 | 2-((4-(2-((4-Cyano-2- fluorobenzyl)oxy)-3- fluorophenyl)piperidin-1- yl)methyl)-4- (difluoromethoxy)-1-methyl- 1H-benzo[d]imidazole-6- carboxylic acid | 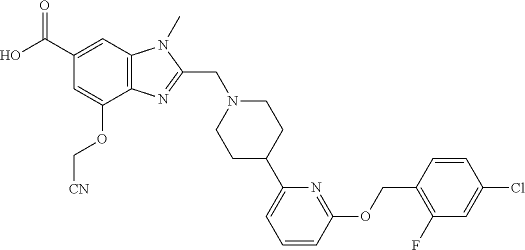 [SMILES: Cn1c(CN2CCC(c3cccc(OCc4ccc(Cl)cc4F)n3)CC2)nc2c(OCC#N)cc(C(=O)O)cc21] |
| 203 | 4-(difluoromethoxy)-1- methyl-2-((4-(6-((4- (trifluoromethyl)benzyl)oxy) pyridin-2-yl)piperidin-1- yl)methyl)-1H- benzo[d]imidazole-6- carboxylic acid | 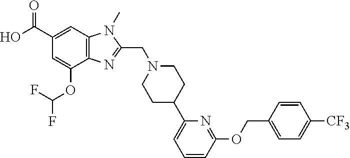 [SMILES: Cn1c(CN2CCC(c3cccc(OCc4ccc(C(F)(F)F)cc4)n3)CC2)nc2c(OC(F)F)cc(C(=O)O)cc21] |
| 204 | 2-((4-(6-((4-Chloro-2- fluorobenzyl)oxy)pyridin-2- yl)piperidin-1-yl)methyl)-7- hydroxy-1H- benzo[d]imidazole-6- carboxylic acid | 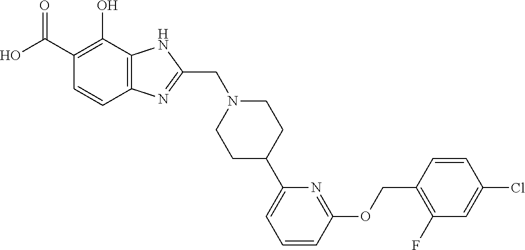 [SMILES: O=C(O)c1ccc2nc(CN3CCC(c4cccc(OCc5ccc(Cl)cc5F)n4)CC3)[nH]c2c1O] |
| 205 | 2-((4-(1-(4-Chloro-2- fluorobenzyl)-1H- pyrrolo[2,3-b]pyridin-6- yl)piperidin-1-yl)methyl)-4- (difluoromethoxy)-1-methyl- 1H-benzo[d]imidazole-6- carboxylic acid | 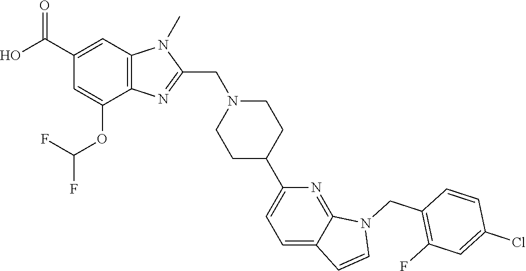 [SMILES: Cn1c(CN2CCC(c3ccc4ccn(Cc5ccc(Cl)cc5F)c4n3)CC2)nc2c(OC(F)F)cc(C(=O)O)cc21] |
| 206 | 2-((4-(6-((4-Chloro-2- fluorobenzyl)oxy)pyridin-2- yl)piperidin-1-yl)methyl)-7- (difluoromethoxy)-1- (fluoromethyl)-1H- benzo[d]imidazole-5- carboxylic acid | 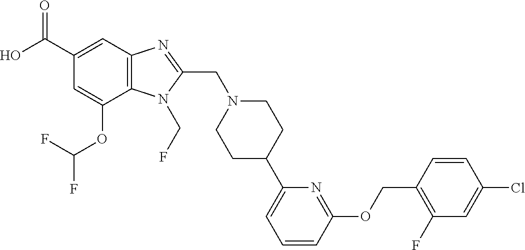 [SMILES: O=C(O)c1cc(OC(F)F)c2c(c1)nc(CN1CCC(c3cccc(OCc4ccc(Cl)cc4F)n3)CC1)n2CF] |
| 207 | 2-((4-(6-((4-chloro-2- fluorobenzyl)oxy)pyridin-2- yl)piperidin-1-yl)methyl)-4- (methoxy-d3)-1-methyl-1H- benzo[d]imidazole-6- carboxylic acid | 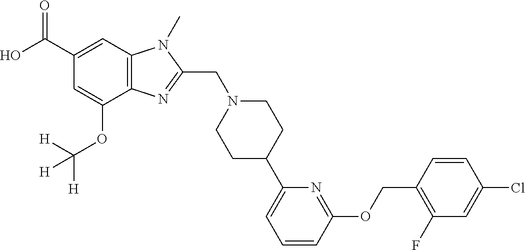 [SMILES: COc1cc(C(=O)O)cc2c1nc(CN1CCC(c3cccc(OCc4ccc(Cl)cc4F)n3)CC1)n2C] |
| 208 | 2-((4-(6-((4-Chloro-2- fluorobenzyl)oxy)pyridin-2- yl)piperidin-1-yl)methyl)-4- (difluoromethoxy)-1- (fluoromethyl)-1H- benzo[d]imidazole-6- carboxylic acid | 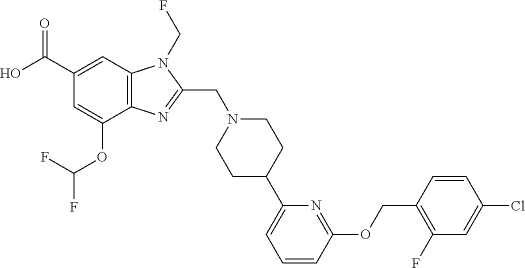 [SMILES: O=C(O)c1cc(OC(F)F)c2nc(CN3CCC(c4cccc(OCc5ccc(Cl)cc5F)n4)CC3)n(CF)c2c1] |
| 209 | 2-((4-(6-((4-Chloro-2- fluorobenzyl)oxy)pyridin-2- yl)piperidin-1-yl)methyl)-1- (fluoromethyl)-4-methoxy- 1H-benzo[d]imidazole-6- carboxylic acid | 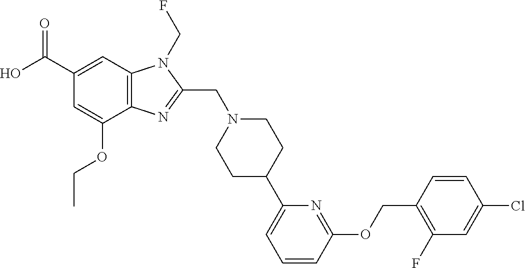 [SMILES: CCOc1cc(C(=O)O)cc2c1nc(CN1CCC(c3cccc(OCc4ccc(Cl)cc4F)n3)CC1)n2CF] |
| 210 | 2-((4-(6-((4-Chloro-2- fluorobenzyl)oxy)pyridin-2- yl)piperidin-1-yl)methyl)-7- (difluoromethoxy)-1H- benzo[d]imidazole-6- carboxylic acid | 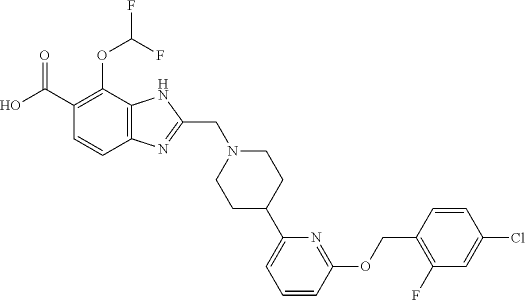 [SMILES: O=C(O)c1ccc2nc(CN3CCC(c4cccc(OCc5ccc(Cl)cc5F)n4)CC3)[nH]c2c1OC(F)F] |
| 211 | (S)-2-((4-(6-((4-Chloro-2- fluorobenzyl)oxy)pyridin-2- yl)piperidin-1-yl)methyl)-1- methyl-4-(oxetan-2- ylmethoxy)-1H- benzo[d]imidazole-6- carboxylic acid | 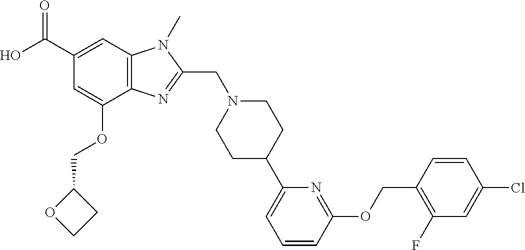 [SMILES: Cn1c(CN2CCC(c3cccc(OCc4ccc(Cl)cc4F)n3)CC2)nc2c(OC[C@@H]3CCO3)cc(C(=O)O)cc21] |
| 212 | 2-((4-(6-((4-Chloro-2-fluoro- 3- methoxybenzyl)oxy)pyridin- 2-yl)piperidin-1-yl)methyl)- 4-(difluoromethoxy)-1- methyl-1H- benzo[d]imidazole-6- carboxylic acid | 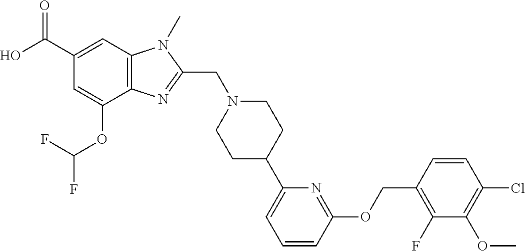 [SMILES: COc1c(Cl)ccc(COc2cccc(C3CCN(Cc4nc5c(OC(F)F)cc(C(=O)O)cc5n4C)CC3)n2)c1F] |
| 213 | 2-((4-(6-((5-Cyanopyridin-2- yl)methoxy)pyridin-2- yl)piperidin-1-yl)methyl)-4- (difluoromethoxy)-1-methyl- 1H-benzo[d]imidazole-6- carboxylic acid | 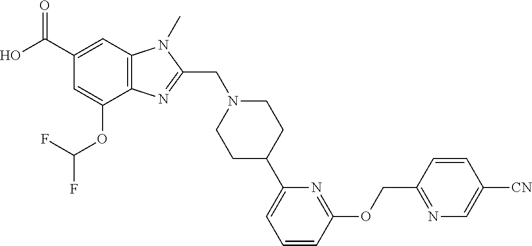 [SMILES: Cn1c(CN2CCC(c3cccc(OCc4ccc(C#N)cn4)n3)CC2)nc2c(OC(F)F)cc(C(=O)O)cc21] |
| 214 | 2-((4-(6-((4-Chloro-2- fluorobenzyl)oxy)pyridin-2- yl)piperidin-1-yl)methyl)-4- ((1- cyanocyclopropyl)methoxy)- 1-methyl-1H- benzo[d]imidazole-6- carboxylic acid | 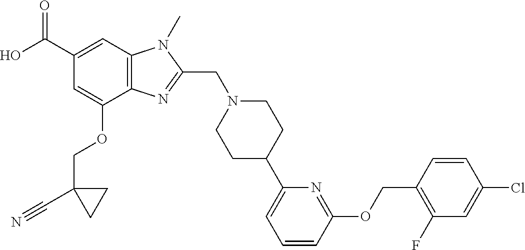 [SMILES: Cn1c(CN2CCC(c3cccc(OCc4ccc(Cl)cc4F)n3)CC2)nc2c(OCC3(C#N)CC3)cc(C(=O)O)cc21] |
| 215 | 2-((4-(6-((4-Chloro-2- fluorobenzyl)oxy)pyridin-2- yl)piperidin-1-yl)methyl)-4- cyclopropoxy-1-methyl-1H- benzo[d]imidazole-6- carboxylic acid | 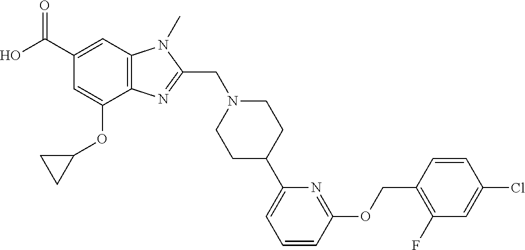 [SMILES: Cn1c(CN2CCC(c3cccc(OCc4ccc(Cl)cc4F)n3)CC2)nc2c(OC3CC3)cc(C(=O)O)cc21] |
| 216 | 2-((4-(3-((4-Chloro-2- fluorobenzyl)oxy)-4- fluorophenyl)piperidin-1- yl)methyl)-4- (difluoromethoxy)-1-methyl- 1H-benzo[d]imidazole-6- carboxylic acid | 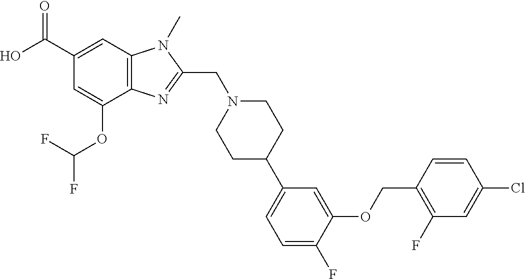 [SMILES: Cn1c(CN2CCC(c3ccc(F)c(OCc4ccc(Cl)cc4F)c3)CC2)nc2c(OC(F)F)cc(C(=O)O)cc21] |
| 217 | 2-((4-(3-((4-Chloro-2- fluorobenzyl)oxy)-1H- pyrazol-1-yl)piperidin-1- yl)methyl)-4- (difluoromethoxy)-1-methyl- 1H-benzo[d]imidazole-6- carboxylic acid | 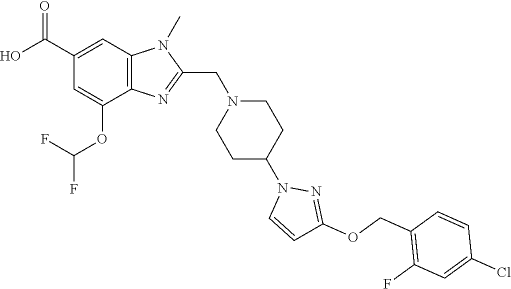 [SMILES: Cn1c(CN2CCC(n3ccc(OCc4ccc(Cl)cc4F)n3)CC2)nc2c(OC(F)F)cc(C(=O)O)cc21] |
| 218 | 4-chloro-2-((4-(6-((4-chloro- 2-fluorobenzyl)oxy)pyridin- 2-yl)piperidin-1-yl)methyl)- 1-methyl-1H- benzo[d]imidazole-6- carboxylic acid | 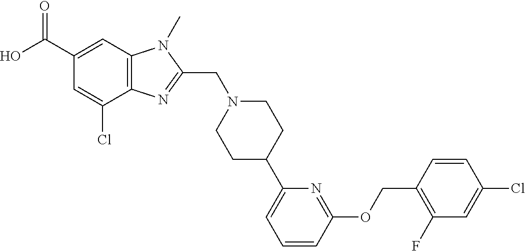 [SMILES: Cn1c(CN2CCC(c3cccc(OCc4ccc(Cl)cc4F)n3)CC2)nc2c(Cl)cc(C(=O)O)cc21] |
| 219 | 2-((4-(6-(4- chlorophenethyl)pyridin-2- yl)piperidin-1-yl)methyl)-4- (difluoromethoxy)-1-methyl- 1H-benzo[d]imidazole-6- carboxylic acid | 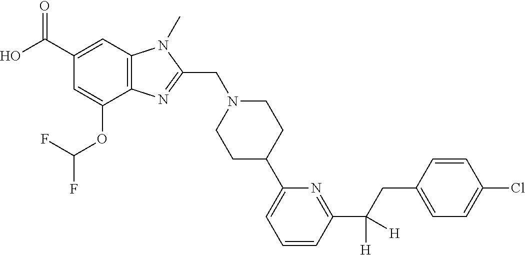 [SMILES: Cn1c(CN2CCC(c3cccc(CCc4ccc(Cl)cc4)n3)CC2)nc2c(OC(F)F)cc(C(=O)O)cc21] |
| 220 | 2-((4-(6-((4-Chloro-2- fluorobenzyl)oxy)pyridin-2- yl)piperidin-1-yl)methyl)-4- (difluoromethoxy)-1-methyl- 1H-benzo[d]imidazole-5- carboxylic acid | 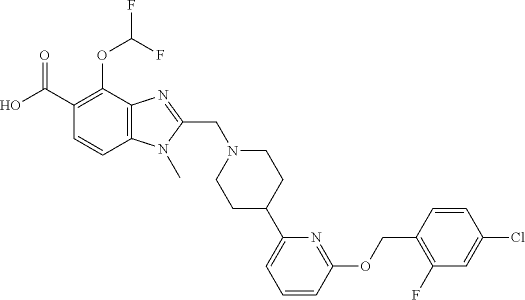 [SMILES: Cn1c(CN2CCC(c3cccc(OCc4ccc(Cl)cc4F)n3)CC2)nc2c(OC(F)F)c(C(=O)O)ccc21] |
| 221 | 4-(Difluoromethoxy)-2-((4- (6-((4-(difluoromethoxy)-2- fluorobenzyl)oxy)pyridin-2- yl)piperidin-1-yl)methyl)-1- methyl-1H- benzo[d]imidazole-6- carboxylic acid | 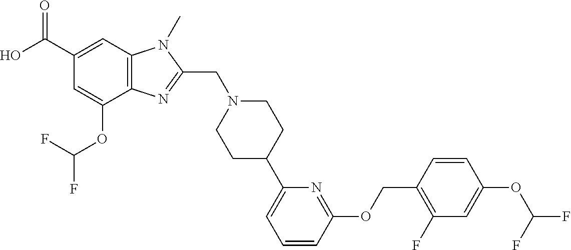 [SMILES: Cn1c(CN2CCC(c3cccc(OCc4ccc(OC(F)F)cc4F)n3)CC2)nc2c(OC(F)F)cc(C(=O)O)cc21] |
| 222 | 4-(Difluoromethoxy)-2-((4- (6-((4-(difluoromethoxy)-2- fluorobenzyl)oxy)pyridin-2- yl)piperidin-1-yl)methyl)-1- methyl-1H- benzo[d]imidazole-6- carboxylic acid | 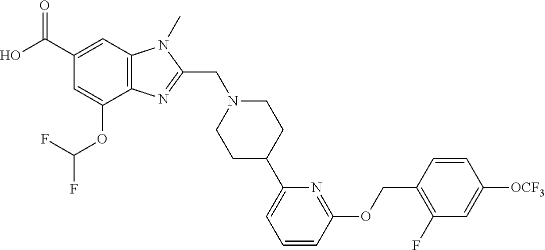 [SMILES: Cn1c(CN2CCC(c3cccc(OCc4ccc(OC(F)(F)F)cc4F)n3)CC2)nc2c(OC(F)F)cc(C(=O)O)cc21] |
| 223 | 4-(Difluoromethoxy)-2-((4- (6-((2-methoxy-4- (trifluoromethyl)benzyl)oxy) pyridin-2-yl)piperidin-1- yl)methyl)-1-methyl-1H- benzo[d]imidazole-6- carboxylic acid | 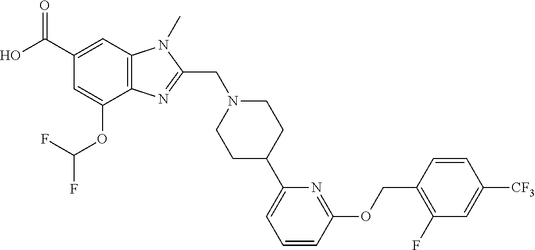 [SMILES: Cn1c(CN2CCC(c3cccc(OCc4ccc(C(F)(F)F)cc4F)n3)CC2)nc2c(OC(F)F)cc(C(=O)O)cc21] |
| 224 | 2-((4-(6-((4-Chloro-2- fluorobenzyl)oxy)pyridin-2- yl)piperidin-1-yl)methyl)-1- methyl-4-(2,2,2- trifluoroethoxy)-1H- benzo[d]imidazole-6- carboxylic acid | 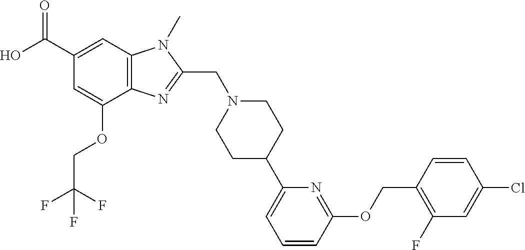 [SMILES: Cn1c(CN2CCC(c3cccc(OCc4ccc(Cl)cc4F)n3)CC2)nc2c(OCC(F)(F)F)cc(C(=O)O)cc21] |
| 225 | 2-((4-(6-((4-Chloro-2- fluorobenzyl)oxy)pyridin-2- yl)piperidin-1-yl)methyl)-4- (fluoromethoxy)-1-methyl- 1H-benzo[d]imidazole-6- carboxylic acid | 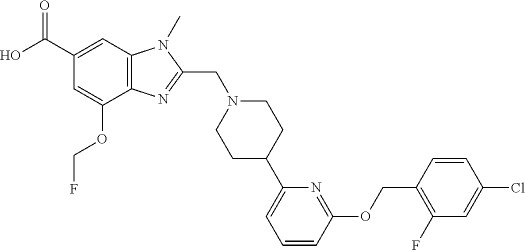 [SMILES: Cn1c(CN2CCC(c3cccc(OCc4ccc(Cl)cc4F)n3)CC2)nc2c(OCF)cc(C(=O)O)cc21] |
| 226 | 2-((4-(6-((4-Chloro-2- methoxybenzyl)oxy)pyridin- 2-yl)piperidin-1-yl)methyl)- 4-(difluoromethoxy)-1- methyl-1H- benzo[d]imidazole-6- carboxylic acid | 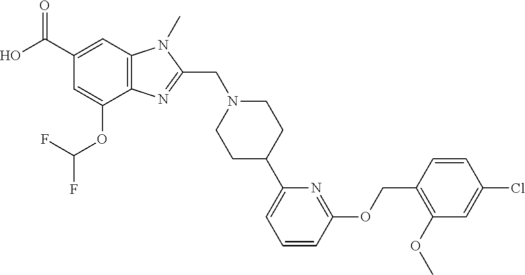 [SMILES: COc1cc(Cl)ccc1COc1cccc(C2CCN(Cc3nc4c(OC(F)F)cc(C(=O)O)cc4n3C)CC2)n1] |
| 227 | 2-((4-(6-((4-Chloro-2- fluorobenzyl)oxy)pyridin-2- yl)piperidin-1-yl)methyl)-4- (1,1-difluoro-2- methoxyethoxy)-1-methyl- 1H-benzo[d]imidazole-6- carboxylic acid | 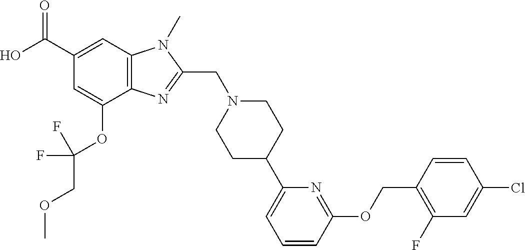 [SMILES: COCC(F)(F)Oc1cc(C(=O)O)cc2c1nc(CN1CCC(c3cccc(OCc4ccc(Cl)cc4F)n3)CC1)n2C] |
| 228 | 4-(Difluoromethoxy)-2-((4- (6-((2-fluoro-4- methoxybenzyl)oxy)pyridin- 2-yl)piperidin-1-yl)methyl)- 1-methyl-1H- benzo[d]imidazole-6- carboxylic acid | 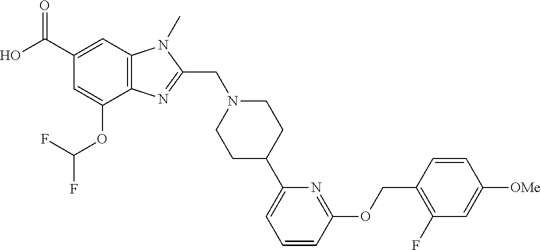 [SMILES: COc1ccc(COc2cccc(C3CCN(Cc4nc5c(OC(F)F)cc(C(=O)O)cc5n4C)CC3)n2)c(F)c1] |
| 229 | 4-(Difluoromethoxy)-1- methyl-2-((4-(6-((5- (trifluoromethyl)pyridin-2- yl)methoxy)pyridin-2- yl)piperidin-1-yl)methyl)- 1H-benzo[d]imidazole-6- carboxylic acid | 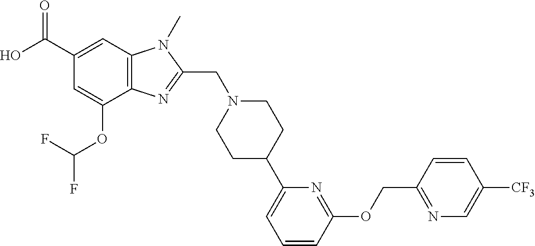 [SMILES: Cn1c(CN2CCC(c3cccc(OCc4ccc(C(F)(F)F)cn4)n3)CC2)nc2c(OC(F)F)cc(C(=O)O)cc21] |
| 230 | 2-((4-(3-((4-Chloro-2- fluorobenzyl)oxy)-4- methoxyphenyl)piperidin-1- yl)methyl)-4- (difluoromethoxy)-1-methyl- 1H-benzo[d]imidazole-6- carboxylic acid | 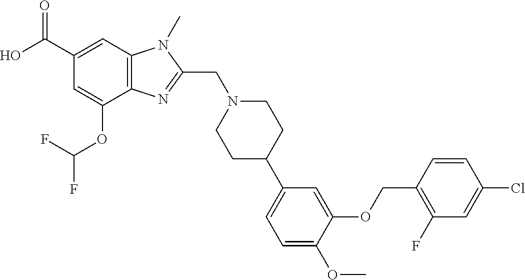 [SMILES: COc1ccc(C2CCN(Cc3nc4c(OC(F)F)cc(C(=O)O)cc4n3C)CC2)cc1OCc1ccc(Cl)cc1F] |
| 231 | 4-(Difluoromethoxy)-2-((4- (6-((4-fluoro-2- methoxybenzyl)oxy)pyridin- 2-yl)piperidin-1-yl)methyl)- 1-methyl-1H- benzo[d]imidazole-6- carboxylic acid | 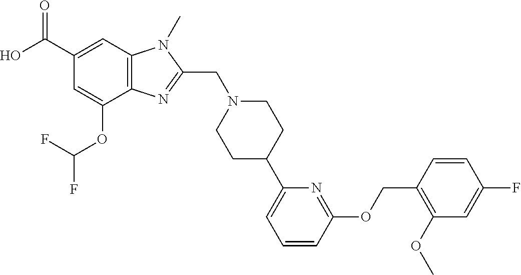 [SMILES: COc1cc(F)ccc1COc1cccc(C2CCN(Cc3nc4c(OC(F)F)cc(C(=O)O)cc4n3C)CC2)n1] |
| 232 | 2-((4-(6-((4-Chloro-2- fluorobenzyl)oxy)pyridin-2- yl)piperidin-1-yl)methyl)-4- (difluoromethoxy)-1-methyl- 6-(5-(trifluoromethyl)-4H- 1,2,4-triazol-3-yl)-1H- benzo[d]imidazole | 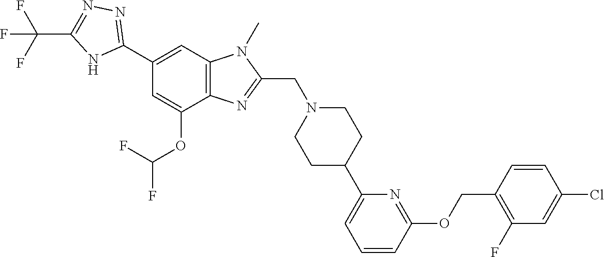 [SMILES: Cn1c(CN2CCC(c3cccc(OCc4ccc(Cl)cc4F)n3)CC2)nc2c(OC(F)F)cc(-c3nnc(C(F)(F)F)[nH]3)cc21] |
| 233 | 2-((4-(6-((4-Chloro-2- fluorobenzyl)oxy)pyridin-2- yl)piperidin-1-yl)methyl)-4- (difluoromethoxy)-1-methyl- 6-(5-(trifluoromethyl)-4H- 1,2,4-triazol-3-yl)-1H- benzo[d]imidazole | 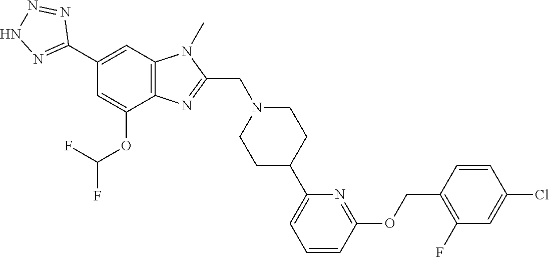 [SMILES: Cn1c(CN2CCC(c3cccc(OCc4ccc(Cl)cc4F)n3)CC2)nc2c(OC(F)F)cc(-c3nn[nH]n3)cc21] |
| 234 | 5-(2-((4-(6-((4-Chloro-2- fluorobenzyl)o midazoldin- 2-yl)piperidin-1-yl)methyl)- 4-(difluoromethoxy)-1- methyl-1H-benzo midazolezol-6-yl)-1,3,4- oxadiazol-2(3H)-one | 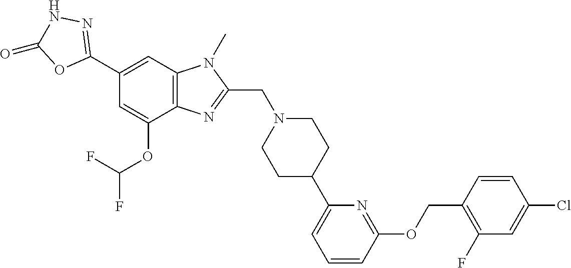 [SMILES: Cn1c(CN2CCC(c3cccc(OCc4ccc(Cl)cc4F)n3)CC2)nc2c(OC(F)F)cc(-c3n[nH]c(=O)o3)cc21] |
| 235 | 2-((4-(6-((4-Chloro-2- fluorobenzyl)oxy)pyridin-2- yl)piperidin-1-yl)methyl)-1- methyl-4-(1,1,2- trifluoroethoxy)-1H- benzo[d]imidazole-6- carboxylic acid | 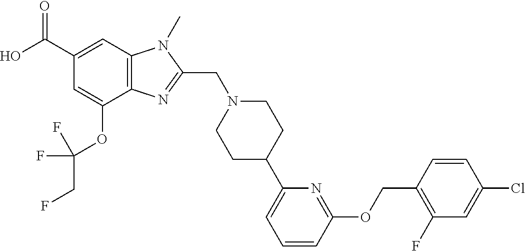 [SMILES: Cn1c(CN2CCC(c3cccc(OCc4ccc(Cl)cc4F)n3)CC2)nc2c(OC(F)(F)CF)cc(C(=O)O)cc21] |
| 236 | 3-(2-((4-(6-((4-Chloro-2- fluorobenzyl)oxy)pyridin-2- yl)piperidin-1-yl)methyl)-4- (difluoromethoxy)-1-methyl- 1H-benzo[d]imidazol-6-yl)- 1,2,4-oxadiazol-5(4H)-one | 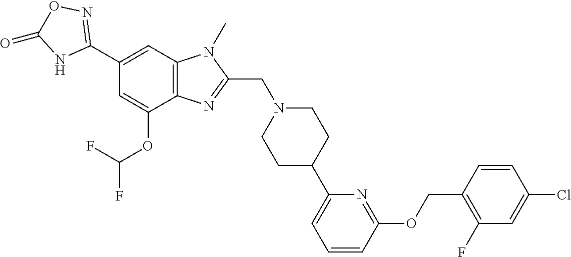 [SMILES: Cn1c(CN2CCC(c3cccc(OCc4ccc(Cl)cc4F)n3)CC2)nc2c(OC(F)F)cc(-c3noc(=O)[nH]3)cc21] |
| 237 | 2-((4-(6-((4- Chlorobenzofuran-7- yl)methoxy)pyridin-2- yl)piperidin-1-yl)methyl)-4- (difluoromethoxy)-1-methyl- 1H-benzo[d]imidazole-6- carboxylic acid | 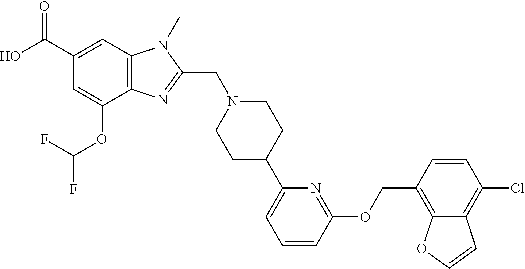 [SMILES: Cn1c(CN2CCC(c3cccc(OCc4ccc(Cl)c5ccoc45)n3)CC2)nc2c(OC(F)F)cc(C(=O)O)cc21] |
| 238 | 4-(Difluoromethoxy)-2-((4- (6-((2-fluoro-4- methylbenzyl)oxy)pyridin-2- yl)piperidin-1-yl)methyl)-1- methyl-1H- benzo[d]imidazole-6- carboxylic acid | 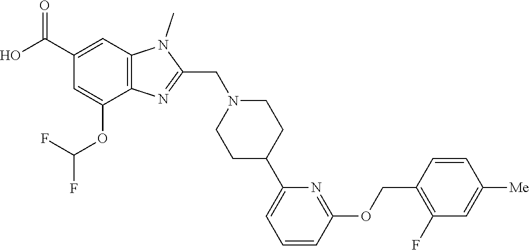 [SMILES: Cc1ccc(COc2cccc(C3CCN(Cc4nc5c(OC(F)F)cc(C(=O)O)cc5n4C)CC3)n2)c(F)c1] |
| 239 | 2-((4-(2-((4-Chloro-2- fluorobenzyl)amino)pyridin- 3-yl)piperidin-1-yl)methyl)- 4-(difluoromethoxy)-1- methyl-1H- benzo[d]imidazole-6- carboxylic acid | 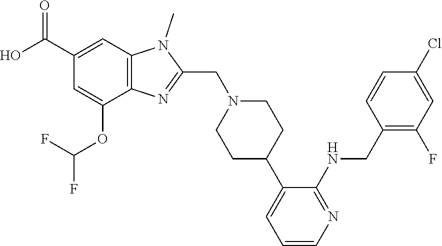 [SMILES: Cn1c(CN2CCC(c3cccnc3NCc3ccc(Cl)cc3F)CC2)nc2c(OC(F)F)cc(C(=O)O)cc21] |
| 240 | (R)-2-((4-(2-(4-chloro-2- fluorophenyl)-4-methyl-3- oxo-3,4-dihydro-2H- benzo[b][1,4]oxazin-8- yl)piperidin-1-yl)methyl)-4- (difluoromethoxy)-1-methyl- 1H-benzo[d]imidazole-6- carboxylic acid | 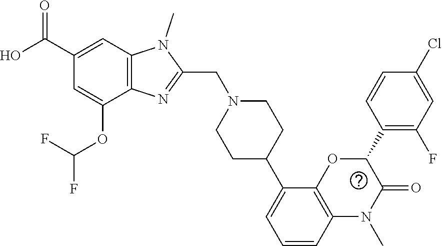 [SMILES: CN1C(=O)[C@@H](c2ccc(Cl)cc2F)Oc2c(C3CCN(Cc4nc5c(OC(F)F)cc(C(=O)O)cc5n4C)CC3)cccc21] |
| 241 | (S)-2-((4-(2-(4-chloro-2- fluorophenyl)-4-methyl-3- oxo-3,4-dihydro-2H- benzo[b][1,4]oxazin-8- yl)piperidin-1-yl)methyl)-4- (difluoromethoxy)-1-methyl- 1H-benzo[d]imidazole-6- carboxylic acid | 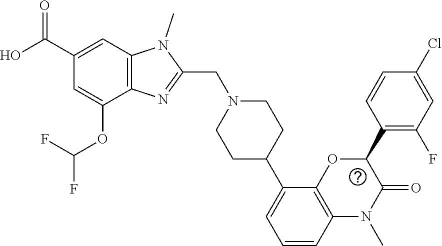 [SMILES: CN1C(=O)[C@H](c2ccc(Cl)cc2F)Oc2c(C3CCN(Cc4nc5c(OC(F)F)cc(C(=O)O)cc5n4C)CC3)cccc21] |
| 242 | 2-((4-(6-((4-Chloro-2- fluorobenzyl)oxy)-5- fluoropyridin-2-yl)piperidin- 1-yl)methyl)-4- (difluoromethoxy)-1-methyl- 1H-benzo[d]imidazole-6- carboxylic acid | 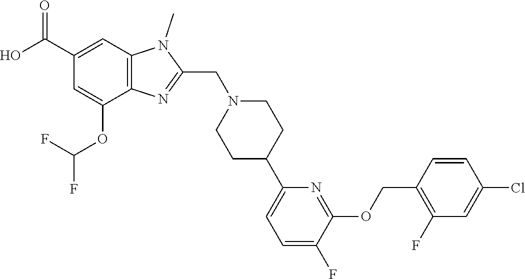 [SMILES: Cn1c(CN2CCC(c3ccc(F)c(OCc4ccc(Cl)cc4F)n3)CC2)nc2c(OC(F)F)cc(C(=O)O)cc21] |
| 243 | 2-((4-(6-((4-Chloro-2- fluorobenzyl)oxy)-3- fluoropyridin-2-yl)piperidin- 1-yl)methyl)-4- (difluoromethoxy)-1-methyl- 1H-benzo[d]imidazole-6- carboxylic acid | 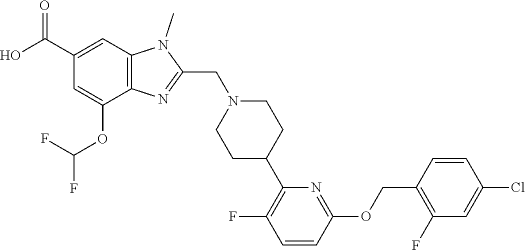 [SMILES: Cn1c(CN2CCC(c3nc(OCc4ccc(Cl)cc4F)ccc3F)CC2)nc2c(OC(F)F)cc(C(=O)O)cc21] |
| 244 | 5-((4-(6-((4-chloro-2- fluorobenzyl)oxy)pyridin-2- yl)piperidin-1-yl)methyl)- 3,4-dihydro-1H-pyrano[4,3- c]quinoline-9-carboxylic acid | 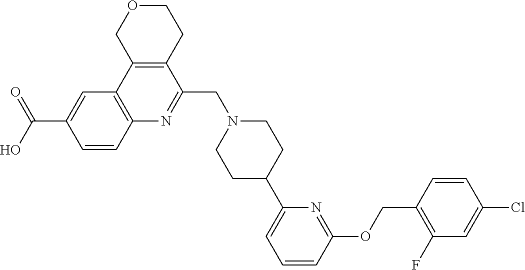 [SMILES: O=C(O)c1ccc2nc(CN3CCC(c4cccc(OCc5ccc(Cl)cc5F)n4)CC3)c3c(c2c1)COCC3] |
| 245 | (S)-2-((4-(6-((4-Chloro-2- fluorobenzyl)oxy)pyridin-2- yl)piperidin-1-yl)methyl)-4- hydroxy-1-(oxetan-2- ylmethyl)-1H- benzo[d]imidazole-5- carboxylic acid | 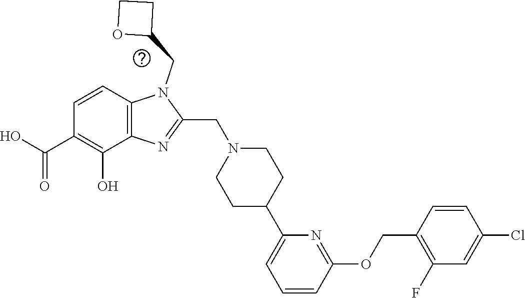 |
| 246 | 2-((4-(6-((4-Chloro-2- (difluoromethoxy)benzyl) oxy)pyridin-2-yl)piperidin-1- yl)methyl)-4- (difluoromethoxy)-1-methyl- 1H-benzo[d]imidazole-6- carboxylic acid | 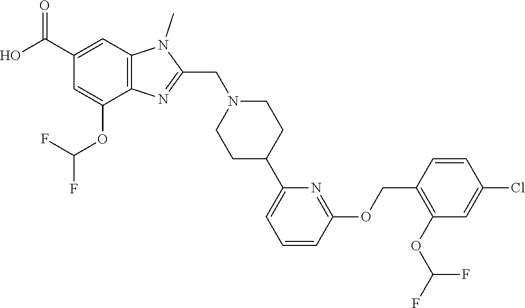 [SMILES: Cn1c(CN2CCC(c3cccc(OCc4ccc(Cl)cc4OC(F)F)n3)CC2)nc2c(OC(F)F)cc(C(=O)O)cc21] |
| 247 | 2-((4-(6-((5-Chloropyridin-2- yl)methoxy)pyridin-2- yl)piperidin-1-yl)methyl)-4- (difluoromethoxy)-1-methyl- 1H-benzo[d]imidazole-6- carboxylic acid | 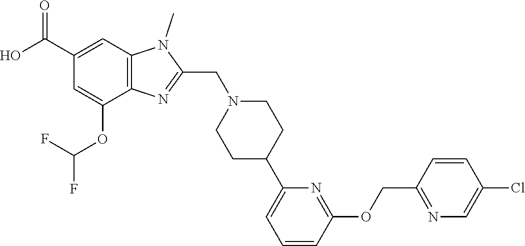 [SMILES: Cn1c(CN2CCC(c3cccc(OCc4ccc(Cl)cn4)n3)CC2)nc2c(OC(F)F)cc(C(=O)O)cc21] |
| 248 | 2-((4-(6-((2- Chlorobenzyl)oxy)pyridin-2- yl)piperidin-1-yl)methyl)-4- (difluoromethoxy)-1-methyl- 1H-benzo[d]imidazole-6- carboxylic acid | 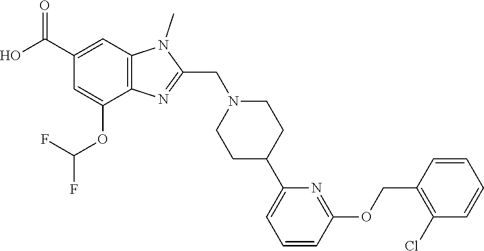 [SMILES: Cn1c(CN2CCC(c3cccc(OCc4ccccc4Cl)n3)CC2)nc2c(OC(F)F)cc(C(=O)O)cc21] |
| 249 | 2-((4-(6-((4-Chloro-2- fluorobenzyl)oxy)pyridin-2- yl)piperidin-1-yl)methyl)-4- methoxy-1-methyl-1H- benzo[d]imidazole-6- carboxylic acid | 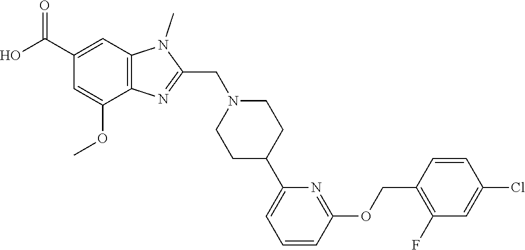 [SMILES: COc1cc(C(=O)O)cc2c1nc(CN1CCC(c3cccc(OCc4ccc(Cl)cc4F)n3)CC1)n2C] |
| 250 | 2-((4-(6-((2,4- Difluorobenzyl)oxy)pyridin- 2-yl)piperidin-1-yl)methyl)- 4-(difluoromethoxy)-1- methyl-1H- benzo[d]imidazole-6- carboxylic acid | 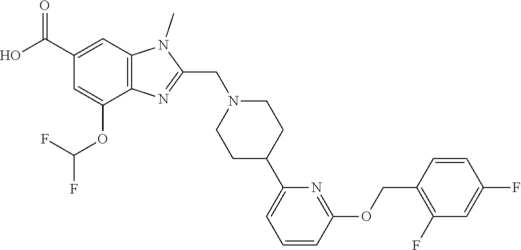 [SMILES: Cn1c(CN2CCC(c3cccc(OCc4ccc(F)cc4F)n3)CC2)nc2c(OC(F)F)cc(C(=O)O)cc21] |
| 251 | 4-(Difluoromethoxy)-2-((4- (6-((2- fluorobenzyl)oxy)pyridin-2- yl)piperidin-1-yl)methyl)-1- methyl-1H- benzo[d]imidazole-6- carboxylic acid | 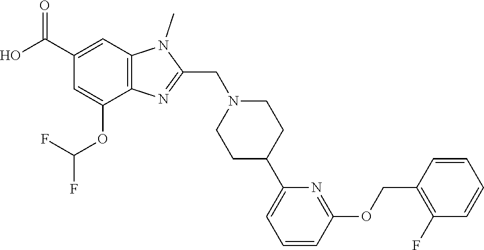 [SMILES: Cn1c(CN2CCC(c3cccc(OCc4ccccc4F)n3)CC2)nc2c(OC(F)F)cc(C(=O)O)cc21] |
| 252 | 2-((4-(6-((4- Chlorobenzyl)oxy)pyridin-2- yl)piperidin-1-yl)methyl)-4- (difluoromethoxy)-1-methyl- 1H-benzo[d]imidazole-6- carboxylic acid | 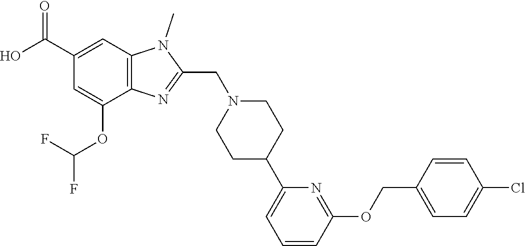 [SMILES: Cn1c(CN2CCC(c3cccc(OCc4ccc(Cl)cc4)n3)CC2)nc2c(OC(F)F)cc(C(=O)O)cc21] |
| 253 | 2-((4-(6-((4-Chloro-2- fluorobenzyl)oxy)pyridin-2- yl)piperidin-1-yl)methyl)-4- (difluoromethoxy)-1-methyl- 1H-imidazo[4,5-c]pyridine- 6-carboxylic acid | 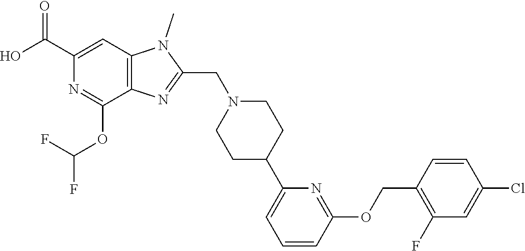 [SMILES: Cn1c(CN2CCC(c3cccc(OCc4ccc(Cl)cc4F)n3)CC2)nc2c(OC(F)F)nc(C(=O)O)cc21] |
| 254 | 4-(Difluoromethoxy)-2-((4- (6-((4- fluorobenzyl)oxy)pyridin-2- yl)piperidin-1-yl)methyl)-1- methyl-1H- benzo[d]imidazole-6- carboxylic acid | 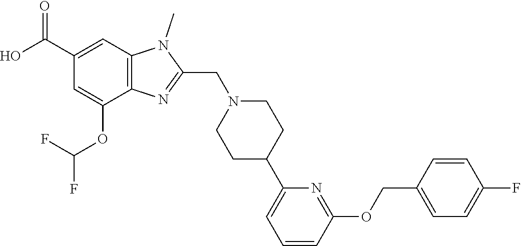 [SMILES: Cn1c(CN2CCC(c3cccc(OCc4ccc(F)cc4)n3)CC2)nc2c(OC(F)F)cc(C(=O)O)cc21] |
| 255 | (2-((4-(6-((4-chloro-2- fluorobenzyl)oxy)pyridin-2- yl)piperidin-1-yl)methyl)-4- (difluoromethoxy)-1-methyl- 1H-benzo[d]imidazol-6- yl)glycine | 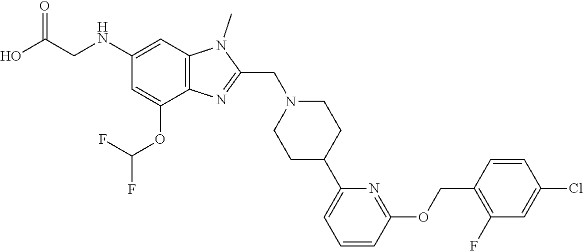 [SMILES: Cn1c(CN2CCC(c3cccc(OCc4ccc(Cl)cc4F)n3)CC2)nc2c(OC(F)F)cc(NCC(=O)O)cc21] |
| 256 | 2-((4-((6-((4-chloro-2- fluorophenoxy)methyl) pyridin-2-yl)oxy)piperidin- 1-yl)methyl)-4-ethoxy-1- methyl-1H- benzo[d]imidazole-6- carboxylic acid | 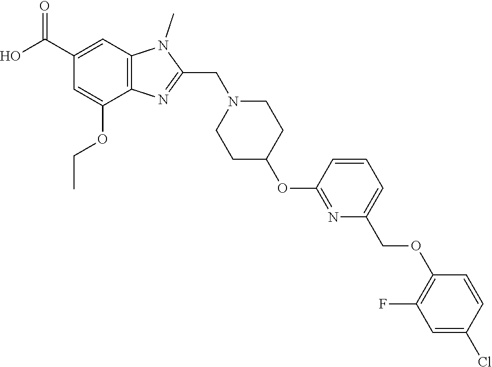 [SMILES: CCOc1cc(C(=O)O)cc2c1nc(CN1CCC(Oc3cccc(COc4ccc(Cl)cc4F)n3)CC1)n2C] |
| 257 | (S)-3-(2-((4-(6-((4-Chloro-2- fluorobenzyl)oxy)pyridin-2- yl)piperidin-1-yl)methyl)-4- methoxy-1-(oxetan-2- ylmethyl)-1H- benzo[d]imidazol-6- yl)propiolic acid | 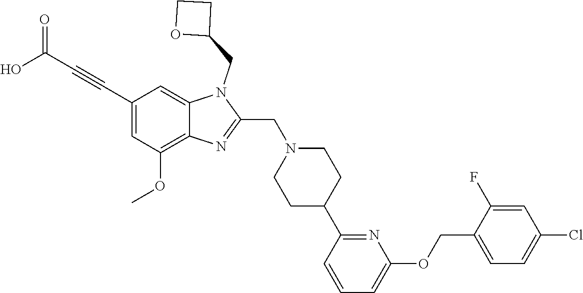 [SMILES: COc1cc(C#CC(=O)O)cc2c1nc(CN1CCC(c3cccc(OCc4ccc(Cl)cc4F)n3)CC1)n2C[C@@H]1CCO1] |
| 258 | (S)-2-((4-(2-(4-Chloro-2- fluorophenyl)-2,3- dihydrobenzo[b][1,4]dioxin- 5-yl)piperidin-1-yl)methyl)- 4-methoxy-1-methyl-1H- benzo[d]imidazole-6- carboxylic acid | 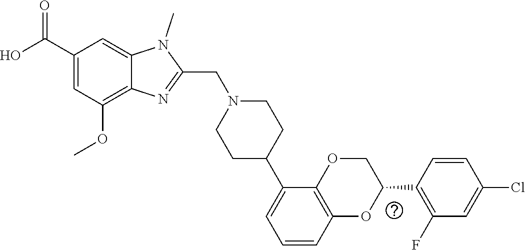 [SMILES: COc1cc(C(=O)O)cc2c1nc(CN1CCC(c3cccc4c3OC[C@H](c3ccc(Cl)cc3F)O4)CC1)n2C] |
| 259 | 2-(2-((4-(6-((4-Cyano-2- fluorobenzyl)oxy)pyridin-2- yl)piperidin-1-yl)methyl)-4- ethoxy-1-methyl-1H- benzo[d]imidazol-6-yl)acetic acid | 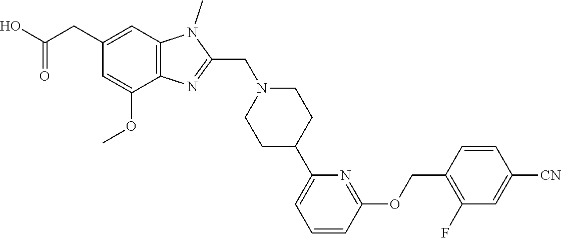 [SMILES: COc1cc(CC(=O)O)cc2c1nc(CN1CCC(c3cccc(OCc4ccc(C#N)cc4F)n3)CC1)n2C] |
| 260 | 4-(((6-(1-((4-ethoxy-1- methyl-6-(5-oxo-2,5- dihydro-1,2,4-oxadiazol-3- yl)-1H-benzo[d]imidazol-2- yl)methyl)piperidin-4- yl)pyridin-2-yl)oxy)methyl)- 3-fluorobenzonitrile | 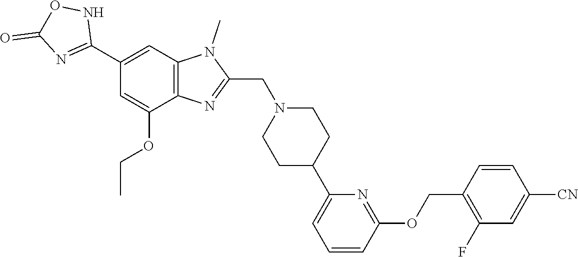 [SMILES: CCOc1cc(-c2nc(=O)o[nH]2)cc2c1nc(CN1CCC(c3cccc(OCc4ccc(C#N)cc4F)n3)CC1)n2C] |
| 261 | 2-((4-(6-((4-Cyano-2- fluorobenzyl)oxy)pyridin-2- yl)piperidin-1-yl)methyl)-4- ethoxy-1-methyl-N- (methylsulfonyl)-1H- benzo[d]imidazole-6- carboxamide | 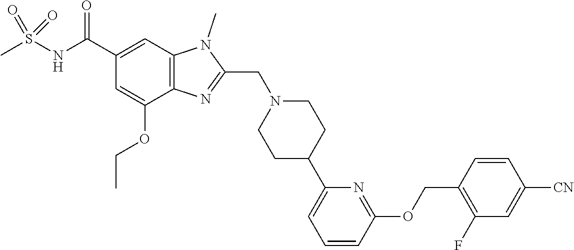 [SMILES: CCOc1cc(C(=O)NS(C)(=O)=O)cc2c1nc(CN1CCC(c3cccc(OCc4ccc(C#N)cc4F)n3)CC1)n2C] |
| 262 | rel-(R)-2-((4-(6-((4-Chloro- 2-fluorobenzyl)oxy)pyridin- 2-yl)piperidin-1-yl)methyl)- 4-((1-fluoropropan-2- yl)oxy)-1-methyl-1H- benzo[d]imidazole-6- carboxylic acid | 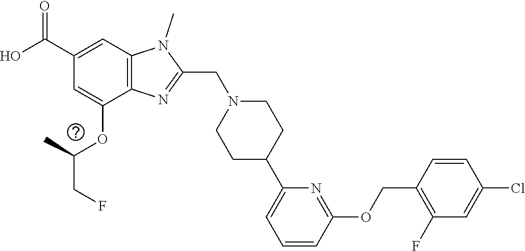 [SMILES: C[C@H](CF)Oc1cc(C(=O)O)cc2c1nc(CN1CCC(c3cccc(OCc4ccc(Cl)cc4F)n3)CC1)n2C] |
| 263 | rel-(S)-2-((4-(6-((4-Chloro- 2-fluorobenzyl)oxy)pyridin- 2-yl)piperidin-1-yl)methyl)- 4-((1-fluoropropan-2- yl)oxy)-1-methyl-1H- benzo[d]imidazole-6- carboxylic acid | 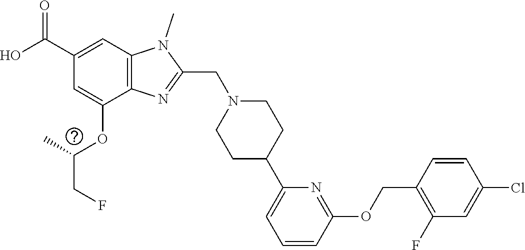 [SMILES: C[C@@H](CF)Oc1cc(C(=O)O)cc2c1nc(CN1CCC(c3cccc(OCc4ccc(Cl)cc4F)n3)CC1)n2C] |
| 264 | (2-((4-(3-((4-chloro-2- fluorobenzyl)oxy)phenyl) piperidin-1-yl)methyl)-4- (difluoromethoxy)-1-methyl- 1H-benzo[d]imidazol-6- yl)glycine | 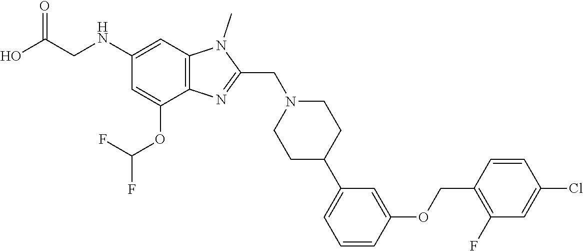 [SMILES: Cn1c(CN2CCC(c3cccc(OCc4ccc(Cl)cc4F)c3)CC2)nc2c(OC(F)F)cc(NCC(=O)O)cc21] |
| 265 | (R)-2-((4-(3-(4-chloro-2- fluorophenyl)-2,3- dihydrobenzo[b][1,4]dioxin- 5-yl)piperidin-1-yl)methyl)- 4-ethoxy-1-methyl-1H- benzo[d]imidazole-6- carboxylic acid | 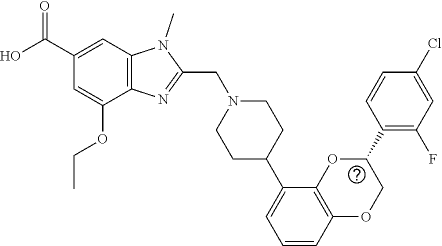 [SMILES: CCOc1cc(C(=O)O)cc2c1nc(CN1CCC(c3cccc4c3O[C@H](c3ccc(Cl)cc3F)CO4)CC1)n2C] |
| 266 | (S)-2-((4-(3-(4-chloro-2- fluorophenyl)-2,3- dihydrobenzo[b][1,4]dioxin- 5-yl)piperidin-1-yl)methyl)- 4-ethoxy-1-methyl-1H- benzo[d]imidazole-6- carboxylic acid | 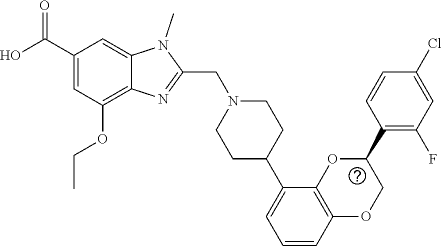 [SMILES: CCOc1cc(C(=O)O)cc2c1nc(CN1CCC(c3cccc4c3O[C@@H](c3ccc(Cl)cc3F)CO4)CC1)n2C] |
| 267 | (R)-2-((4-(3-(4-Chloro-2- fluorophenyl)-2,3- dihydrobenzo[b][1,4]dioxin- 5-yl)piperidin-1-yl)methyl)- 4-methoxy-1-methyl-1H- benzo[d]imidazole-6- carboxylic acid | 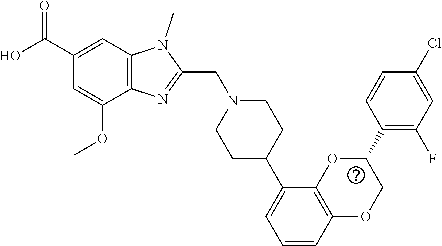 [SMILES: COc1cc(C(=O)O)cc2c1nc(CN1CCC(c3cccc4c3O[C@H](c3ccc(Cl)cc3F)CO4)CC1)n2C] |
| 268 | (S)-2-((4-(3-(4-Chloro-2- fluorophenyl)-2,3- dihydrobenzo[b][1,4]dioxin- 5-yl)piperidin-1-yl)methyl)- 4-methoxy-1-methyl-1H- benzo[d]imidazole-6- carboxylic acid | 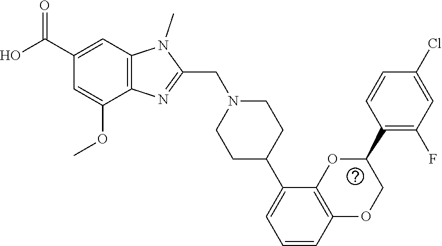 [SMILES: COc1cc(C(=O)O)cc2c1nc(CN1CCC(c3cccc4c3O[C@@H](c3ccc(Cl)cc3F)CO4)CC1)n2C] |
| 269 | (R)-2-((4-(2-(4-chloro-2- fluorophenyl)-2,3- dihydrobenzo[b][1,4]dioxin- 5-yl)piperidin-1-yl)methyl)- 4-ethoxy-1-methyl-1H- benzo[d]imidazole-6- carboxylic acid | 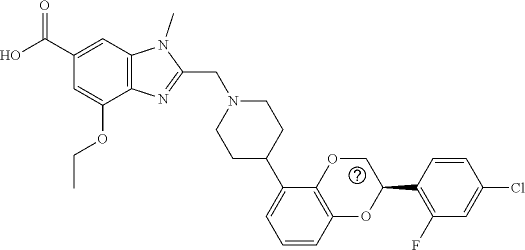 [SMILES: CCOc1cc(C(=O)O)cc2c1nc(CN1CCC(c3cccc4c3OC[C@@H](c3ccc(Cl)cc3F)O4)CC1)n2C] |
| 270 | (S)-2-((4-(2-(4-Chloro-2- fluorophenyl)-2,3- dihydrobenzo[b][1,4]dioxin- 5-yl)piperidin-1-yl)methyl)- 4-ethoxy-1-methyl-1H- benzo[d]imidazole-6- carboxylic acid | 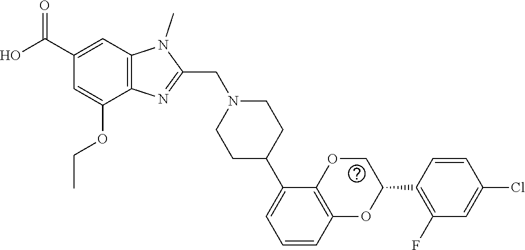 [SMILES: CCOc1cc(C(=O)O)cc2c1nc(CN1CCC(c3cccc4c3OC[C@H](c3ccc(Cl)cc3F)O4)CC1)n2C] |
| 271 | (R)-2-((4-(2-(4-Chloro-2- fluorophenyl)-2,3- dihydrobenzo[b][1,4]dioxin- 5-yl)piperidin-1-yl)methyl)- 4-methoxy-1-methyl-1H- benzo[d]imidazole-6- carboxylic acid | 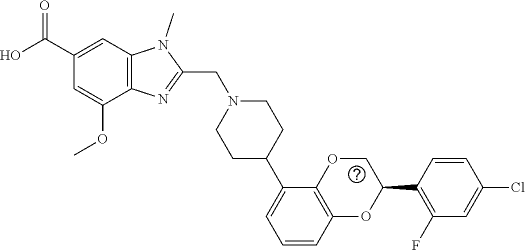 [SMILES: COc1cc(C(=O)O)cc2c1nc(CN1CCC(c3cccc4c3OC[C@@H](c3ccc(Cl)cc3F)O4)CC1)n2C] |
| 272 | 2-((4-(6-((4-Chloro-2,5- difluorobenzyl)oxy)pyridin- 2-yl)piperidin-1-yl)methyl)- 4-methoxy-1-methyl-1H- benzo[d]imidazole-6- carboxylic acid | 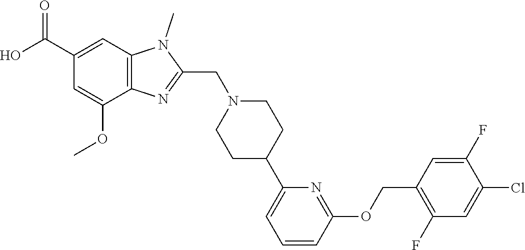 [SMILES: COc1cc(C(=O)O)cc2c1nc(CN1CCC(c3cccc(OCc4cc(F)c(Cl)cc4F)n3)CC1)n2C] |
| 273 | 4-(((6-(1-((4-ethoxy-1- methyl-6-(1H-tetrazol-5-yl)- 1H-benzo[d]imidazol-2- yl)methyl)piperidin-4- yl)pyridin-2-yl)oxy)methyl)- 3-fluorobenzonitrile | 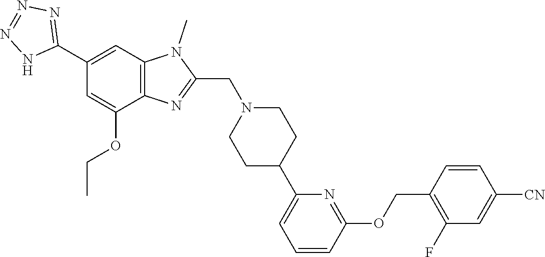 [SMILES: CCOc1cc(-c2nnn[nH]2)cc2c1nc(CN1CCC(c3cccc(OCc4ccc(C#N)cc4F)n3)CC1)n2C] |
| 274 | (S)-2-((4-(2-(4-Cyano-2- fluorophenyl)-2,3- dihydrobenzo[b][1,4]dioxin- 5-yl)piperidin-1-yl)methyl)- 4-methoxy-1-methyl-1H- benzo[d]imidazole-6- carboxylic acid | 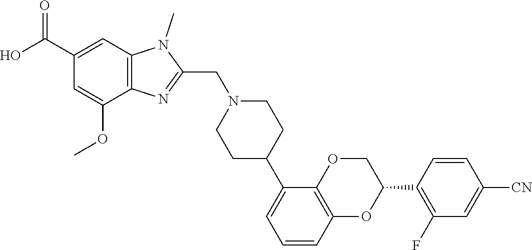 [SMILES: COc1cc(C(=O)O)cc2c1nc(CN1CCC(c3cccc4c3OC[C@H](c3ccc(C#N)cc3F)O4)CC1)n2C] |
| 275 | 7-((4-(6-((4-Chloro-2- fluorobenzyl)oxy)pyridin-2- yl)piperidin-1-yl)methyl)-6- methyl-6H- [1,3]dioxolo[4′,5′:3,4]benzo [1,2-d]imidazole-4-carboxylic acid | 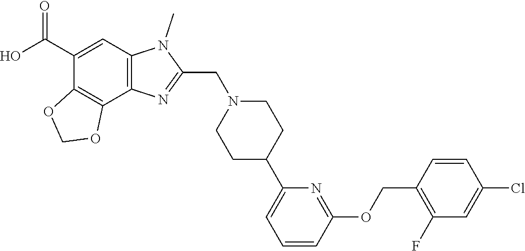 [SMILES: Cn1c(CN2CCC(c3cccc(OCc4ccc(Cl)cc4F)n3)CC2)nc2c3c(c(C(=O)O)cc21)OCO3] |
| 276 | (R)-2-((4-(2-(4-Cyano-2- fluorophenyl)-2,3- dihydrobenzo[b][1,4]dioxin- 5-yl)piperidin-1-yl)methyl)- 4-ethoxy-1-methyl-1H- benzo[d]imidazole-6- carboxylic acid | 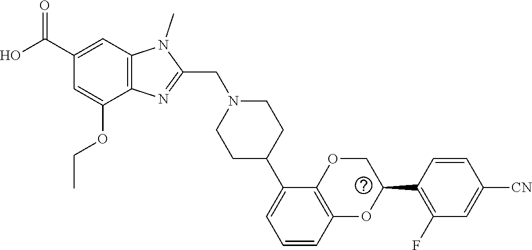 [SMILES: CCOc1cc(C(=O)O)cc2c1nc(CN1CCC(c3cccc4c3OC[C@@H](c3ccc(C#N)cc3F)O4)CC1)n2C] |
| 277 | (S)-2-((4-(2-(4-Cyano-2- fluorophenyl)-2,3- dihydrobenzo[b][1,4]dioxin- 5-yl)piperidin-1-yl)methyl)- 4-ethoxy-1-methyl-1H- benzo[d]imidazole-6- carboxylic acid | 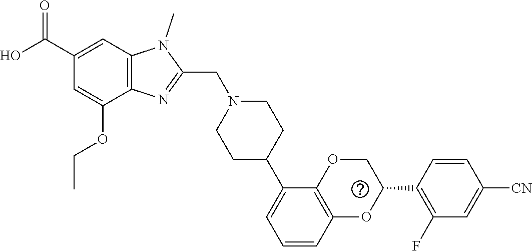 [SMILES: CCOc1cc(C(=O)O)cc2c1nc(CN1CCC(c3cccc4c3OC[C@H](c3ccc(C#N)cc3F)O4)CC1)n2C] |
| 278 | (R)-2-((4-(2-(4-Cyano-2- fluorophenyl)-2,3- dihydrobenzo[b][1,4]dioxin- 5-yl)piperidin-1-yl)methyl)- 4-methoxy-1-methyl-1H- benzo[d]imidazole-6- carboxylic acid | 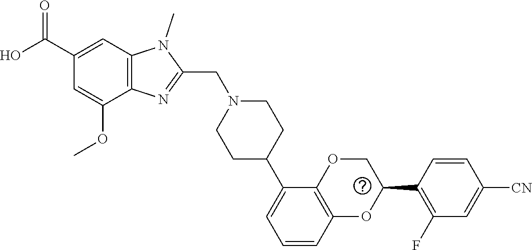 [SMILES: COc1cc(C(=O)O)cc2c1nc(CN1CCC(c3cccc4c3OC[C@@H](c3ccc(C#N)cc3F)O4)CC1)n2C] |
| 279 | (S)-2-((4-(6-((4-Chloro-2- fluorobenzyl)oxy)pyridin-2- yl)piperidin-1-yl)methyl)-4- methoxy-1-(oxetan-2- ylmethyl)-6-(1H-tetrazol-5- yl)-1H-benzo[d]imidazole | 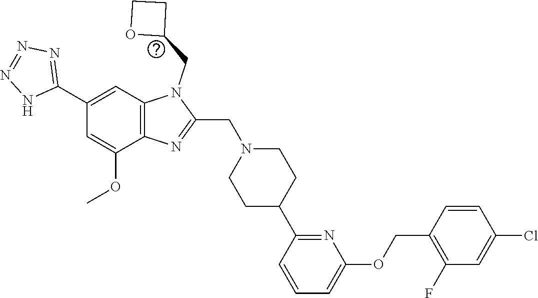 [SMILES: COc1cc(-c2nnn[nH]2)cc2c1nc(CN1CCC(c3cccc(OCc4ccc(Cl)cc4F)n3)CC1)n2C[C@@H]1CCO1] |
| 280 | 2-((4-(6-((4-chloro-2- fluorobenzyl)oxy)pyridin-2- yl)piperidin-1-yl)methyl)-4- methoxy-N, 1-dimethyl-1H- benzo[d]imidazole-6- carboxamide | 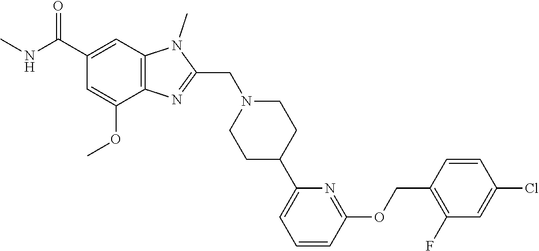 [SMILES: CNC(=O)c1cc(OC)c2nc(CN3CCC(c4cccc(OCc5ccc(Cl)cc5F)n4)CC3)n(C)c2c1] |
| 281 | 5-(2-((4-(3-((4-Chloro-2- fluorobenzyl)oxy)phenyl) piperidin-1-yl)methyl)-4- (difluoromethoxy)-1-methyl- 1H-benzo[d]imidazol-6-yl)- 1,2,5-thiadiazolidin-3-one 1,1-dioxide | 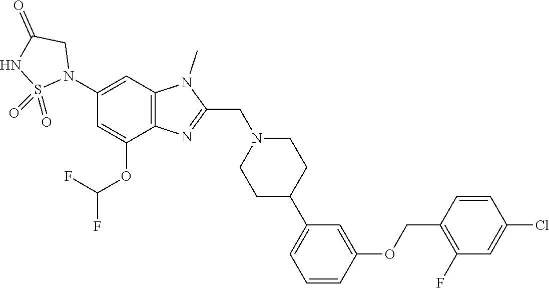 [SMILES: Cn1c(CN2CCC(c3cccc(OCc4ccc(Cl)cc4F)c3)CC2)nc2c(OC(F)F)cc(N3CC(=O)NS3(=O)=O)cc21] |
| 282 | 2-(4-(6-((4-Chloro-2- fluorobenzyl)oxy)pyridin-2- yl)-2,5-difluorobenzyl)-4- methoxy-1-methyl-1H- benzo[d]imidazole-6- carboxylic acid | 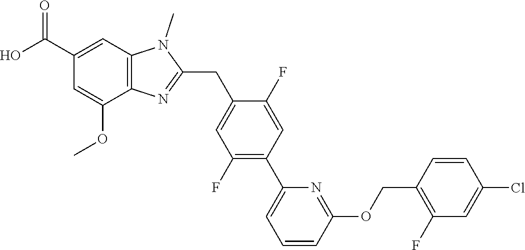 [SMILES: COc1cc(C(=O)O)cc2c1nc(Cc1cc(F)c(-c3cccc(OCc4ccc(Cl)cc4F)n3)cc1F)n2C] |
| 283 | 2-((4-(6-((4-Cyano-2,3- dihydrobenzofuran-7- yl)methoxy)pyridin-2- yl)piperidin-1-yl)methyl)-4- methoxy-1-methyl-1H- benzo[d]imidazole-6- carboxylic acid | 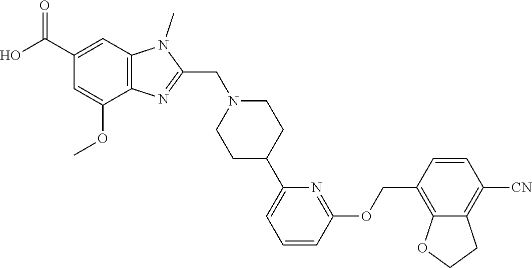 [SMILES: COc1cc(C(=O)O)cc2c1nc(CN1CCC(c3cccc(OCc4ccc(C#N)c5c4OCC5)n3)CC1)n2C] |
| 284 | 2-((4-(3-((4-Chloro-2- fluorobenzyl)oxy)phenyl) piperidin-1-yl)methyl)-4- (ethoxy-1,1-d2)-1-methyl- 1H-benzo[d]imidazole-6- carboxylic acid | 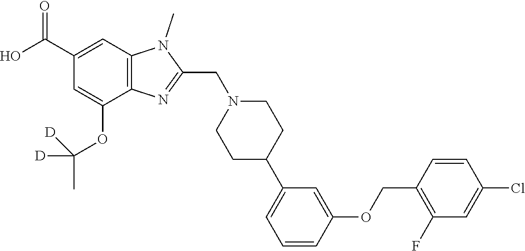 [SMILES: [2H]C([2H])(C)Oc1cc(C(=O)O)cc2c1nc(CN1CCC(c3cccc(OCc4ccc(Cl)cc4F)c3)CC1)n2C] |
| 285 | 2-((4-(6-((4-Cyano-2- fluorophenyl)methoxy- d2)pyridin-2-yl)piperidin-1- yl)methyl)-4-(ethoxy-1,1- d2)-1-methyl-1H- benzo[d]imidazole-6- carboxylic acid | 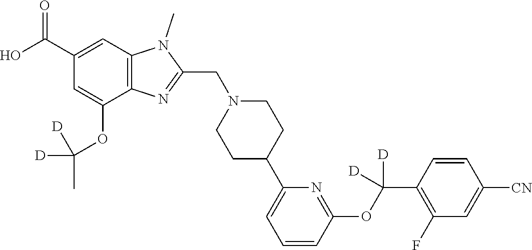 [SMILES: [2H]C([2H])(C)Oc1cc(C(=O)O)cc2c1nc(CN1CCC(c3cccc(OC([2H])([2H])c4ccc(C#N)cc4F)n3)CC1)n2C] |
| 286 | 2-((4-(6-((4-Chloro-2- fluorophenyl)methoxy- d2)pyridin-2-yl)piperidin-1- yl)methyl)-4-(ethoxy-1,1- d2)-1-methyl-1H- benzo[d]imidazole-6- carboxylic acid | 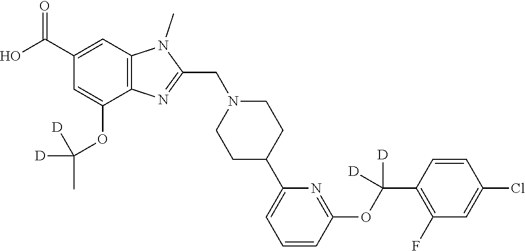 [SMILES: [2H]C([2H])(C)Oc1cc(C(=O)O)cc2c1nc(CN1CCC(c3cccc(OC([2H])([2H])c4ccc(Cl)cc4F)n3)CC1)n2C] |
| 287 | (S)-2-((4-(3-(4-Cyano-2- fluorophenyl)-2,3- dihydrobenzo[b][1,4]dioxin- 5-yl)piperidin-1-yl)methyl)- 4-ethoxy-1-methyl-1H- benzo[d]imidazole-6- carboxylic acid | 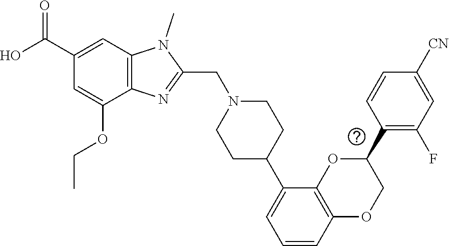 [SMILES: CCOc1cc(C(=O)O)cc2c1nc(CN1CCC(c3cccc4c3O[C@@H](c3ccc(C#N)cc3F)CO4)CC1)n2C] |
| 288 | (R)-2-((4-(3-(4-Cyano-2- fluorophenyl)-2,3- dihydrobenzo[b][1,4]dioxin- 5-yl)piperidin-1-yl)methyl)- 4-ethoxy-1-methyl-1H- benzo[d]imidazole-6- carboxylic acid | 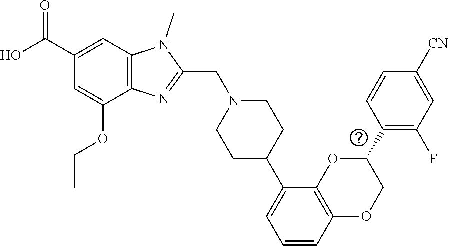 [SMILES: CCOc1cc(C(=O)O)cc2c1nc(CN1CCC(c3cccc4c3O[C@H](c3ccc(C#N)cc3F)CO4)CC1)n2C] |
| 289 | 2-((4-(6-((4-Cyano-2- fluorobenzyl)oxy)pyridin-2- yl)piperidin-1-yl)methyl)-3- methyl-7,8-dihydro-3H- [1,4]dioxino[2′,3′:3,4]benzo [1,2-d]imidazole-5- carboxylic acid | 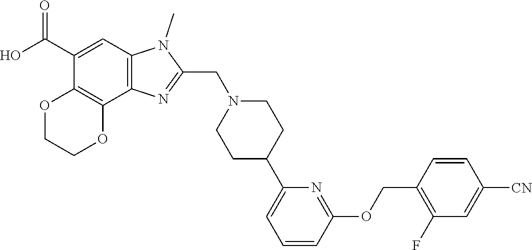 [SMILES: Cn1c(CN2CCC(c3cccc(OCc4ccc(C#N)cc4F)n3)CC2)nc2c3c(c(C(=O)O)cc21)OCCO3] |
| 290 | 2-((4-((6-((4-Cyano-2- fluorophenoxy)methyl) pyridin-2-yl)oxy)piperidin-1- yl)methyl)-4-ethoxy-1- methyl-1H- benzo[d]imidazole-6- carboxylic acid | 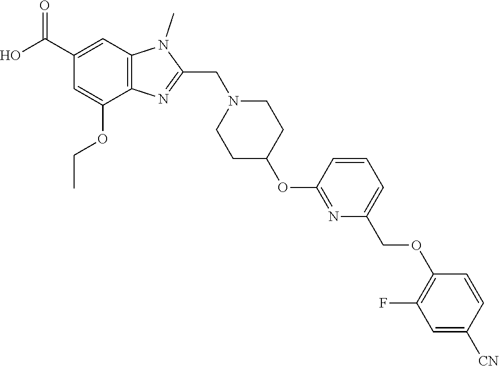 [SMILES: CCOc1cc(C(=O)O)cc2c1nc(CN1CCC(Oc3cccc(COc4ccc(C#N)cc4F)n3)CC1)n2C] |
| 291 | 2-((4-(6-((4-Cyano-2- fluorobenzyl)oxy)pyridin-2- yl)piperidin-1-yl)methyl)-4- (2-hydroxyethoxy)-1-methyl- 1H-benzo[d]imidazole-6- carboxylic acid | 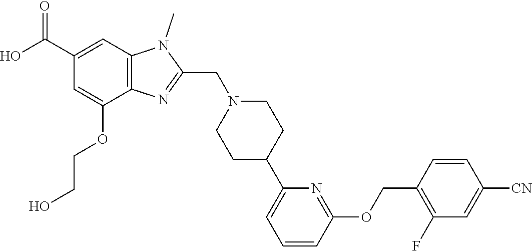 [SMILES: Cn1c(CN2CCC(c3cccc(OCc4ccc(C#N)cc4F)n3)CC2)nc2c(OCCO)cc(C(=O)O)cc21] |
| 292 | 2-(4-(6-((4-Chloro-2- fluorobenzyl)oxy)pyridin-2- yl)-2-fluorobenzyl)-4- methoxy-1-methyl-1H- benzo[d]imidazole-6- carboxylic acid | 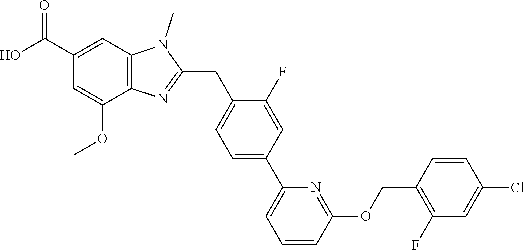 [SMILES: COc1cc(C(=O)O)cc2c1nc(Cc1ccc(-c3cccc(OCc4ccc(Cl)cc4F)n3)cc1F)n2C] |
| 293 | 2-(4-(6-((4-Cyano-2- fluorobenzyl)oxy)pyridin-2- yl)-2-fluorobenzyl)-4- ethoxy-1-methyl-1H- benzo[d]imidazole-6- carboxylic acid | 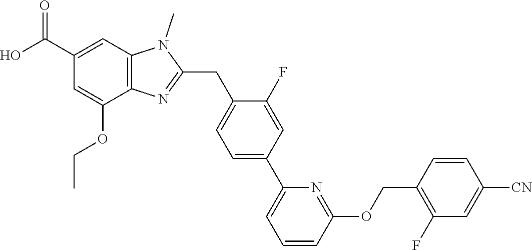 [SMILES: CCOc1cc(C(=O)O)cc2c1nc(Cc1ccc(-c3cccc(OCc4ccc(C#N)cc4F)n3)cc1F)n2C] |
| 294 | (S)-2-((4-(3-(4-Cyano-2- fluorophenyl)-2,3- dihydrobenzo[b][1,4]dioxin- 5-yl)piperidin-1-yl)methyl)- 4-(2-fluoroethoxy)-1-methyl- 1H-benzo[d]imidazole-6- carboxylic acid | 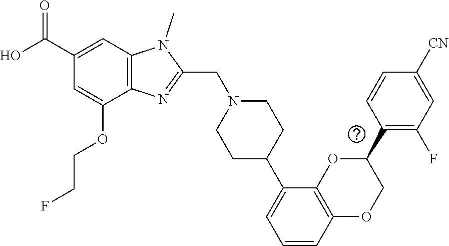 [SMILES: Cn1c(CN2CCC(c3cccc4c3O[C@@H](c3ccc(C#N)cc3F)CO4)CC2)nc2c(OCCF)cc(C(=O)O)cc21] |
| 295 | 2-((4-(6-((4-Cyano-2- fluorobenzyl)oxy)pyridin-2- yl)piperidin-1-yl)methyl)-4- (ethoxy-1,1-d2)-1-methyl- 1H-benzo[d]imidazole-6- carboxylic acid | 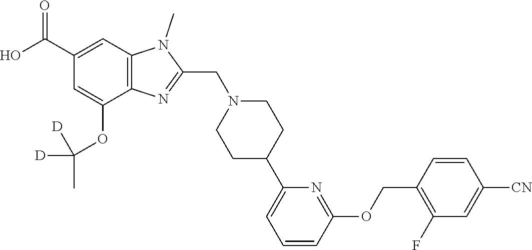 [SMILES: [2H]C([2H])(C)Oc1cc(C(=O)O)cc2c1nc(CN1CCC(c3cccc(OCc4ccc(C#N)cc4F)n3)CC1)n2C] |
| 296 | 2-((4-(6-((4-Chloro-2- fluorobenzyl)oxy)pyridin-2- yl)piperidin-1-yl)methyl)-4- (ethoxy-1,1-d2)-1-methyl- 1H-benzo[d]imidazole-6- carboxylic acid | 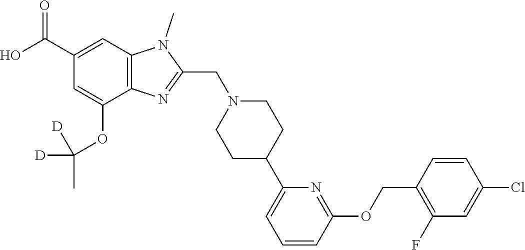 [SMILES: [2H]C([2H])(C)Oc1cc(C(=O)O)cc2c1nc(CN1CCC(c3cccc(OCc4ccc(Cl)cc4F)n3)CC1)n2C] |
| 297 | 2-((4-(6-((4-Chloro-2,3- dihydrobenzofuran-7- yl)methoxy)pyridin-2- yl)piperidin-1-yl)methyl)-4- methoxy-1-methyl-1H- benzo[d]imidazole-6- carboxylic acid | 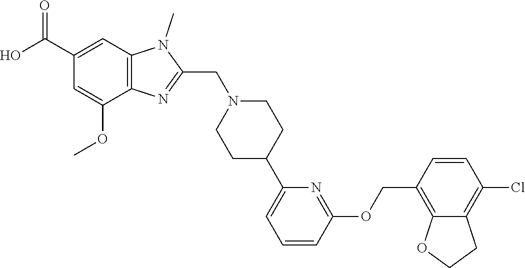 [SMILES: COc1cc(C(=O)O)cc2c1nc(CN1CCC(c3cccc(OCc4ccc(Cl)c5c4OCC5)n3)CC1)n2C] |
| 298 | 2-((4-(6-((4-Cyano-2- fluorobenzyl)oxy)pyridin-2- yl)piperidin-1-yl)methyl)-4- ethoxy-1-methyl-1H- benzo[d]imidazole-6- carboxylic acid | 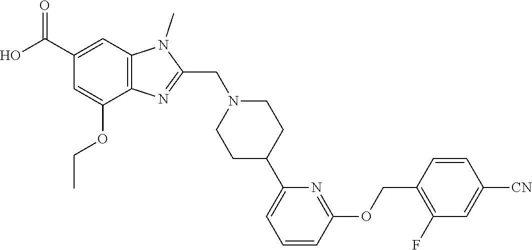 [SMILES: CCOc1cc(C(=O)O)cc2c1nc(CN1CCC(c3cccc(OCc4ccc(C#N)cc4F)n3)CC1)n2C] |
| 299 | (S)-2-((4-(3-(4-chloro-2- fluorophenyl)-2,3- dihydrobenzo[b][1,4]dioxin- 5-yl)piperidin-1-yl)methyl)- 4-(2-fluoroethoxy)-1-methyl- 1H-benzo[d]imidazole-6- carboxylic acid | 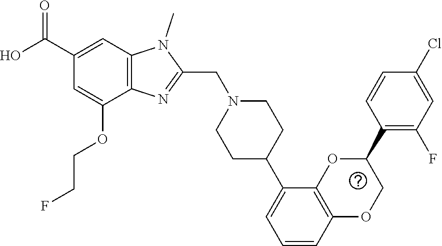 [SMILES: Cn1c(CN2CCC(c3cccc4c3O[C@@H](c3ccc(Cl)cc3F)CO4)CC2)nc2c(OCCF)cc(C(=O)O)cc21] |
| 300 | (R)-2-((4-(2-(4-Cyano-2- fluorophenyl)-2,3- dihydrobenzo[b][1,4]dioxin- 5-yl)piperidin-1-yl)methyl)- 4-(difluoromethoxy)-1- methyl-1H- benzo[d]imidazole-6- carboxylic acid | 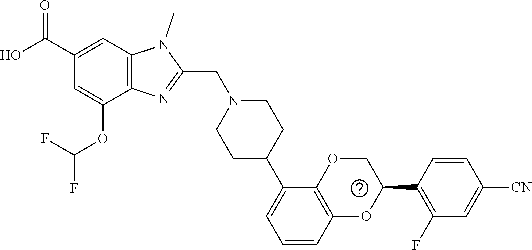 [SMILES: Cn1c(CN2CCC(c3cccc4c3OC[C@@H](c3ccc(C#N)cc3F)O4)CC2)nc2c(OC(F)F)cc(C(=O)O)cc21] |
| 301 | 2-((4-(6-((4-Cyano-2- fluorobenzyl)oxy)pyridin-2- yl)piperidin-1-yl)methyl)-5- fluoro-4-methoxy-1-methyl- 1H-benzo[d]imidazole-6- carboxylic acid | 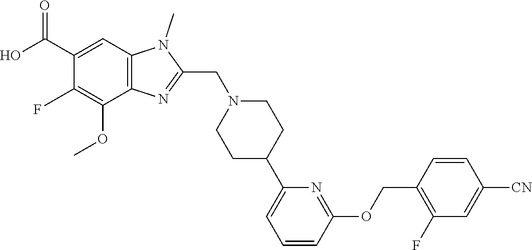 [SMILES: COc1c(F)c(C(=O)O)cc2c1nc(CN1CCC(c3cccc(OCc4ccc(C#N)cc4F)n3)CC1)n2C] |
| 302 | 2-((4-(6-((4-Chloro-2- fluorobenzyl)oxy)pyridin-2- yl)piperidin-1-yl)methyl)-7- fluoro-4-methoxy-1-methyl- 1H-benzo[d]imidazole-6- carboxylic acid | 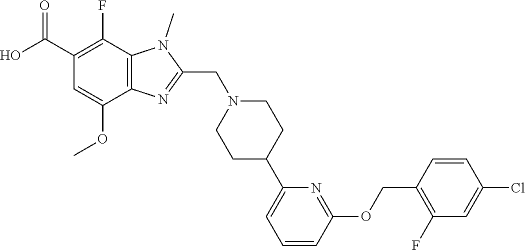 [SMILES: COc1cc(C(=O)O)c(F)c2c1nc(CN1CCC(c3cccc(OCc4ccc(Cl)cc4F)n3)CC1)n2C] |
| 303 | 2-((4-(6-((4-Cyano-2- fluorobenzyl)oxy)pyridin-2- yl)piperidin-1-yl)methyl)-7- fluoro-4-methoxy-1-methyl- 1H-benzo[d]imidazole-6- carboxylic acid | 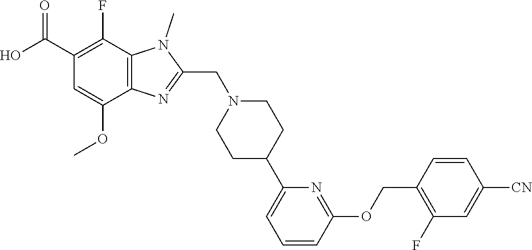 [SMILES: COc1cc(C(=O)O)c(F)c2c1nc(CN1CCC(c3cccc(OCc4ccc(C#N)cc4F)n3)CC1)n2C] |
| 304 | 2-(4-(6-((4-Cyano-2- fluorobenzyl)oxy)pyridin-2- yl)-2,5-difluorobenzyl)-4- methoxy-1-methyl-1H- benzo[d]imidazole-6- carboxylic acid | 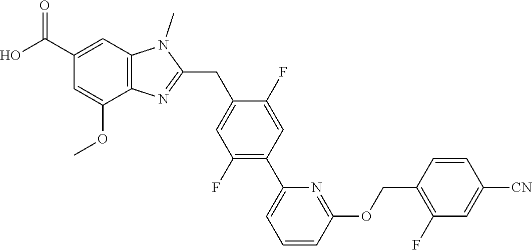 [SMILES: COc1cc(C(=O)O)cc2c1nc(Cc1cc(F)c(-c3cccc(OCc4ccc(C#N)cc4F)n3)cc1F)n2C] |
| 305 | 2-((4-(6-((4- (Difluoromethyl)-2- fluorobenzyl)oxy)pyridin-2- yl)piperidin-1-yl)methyl)-4- methoxy-1-methyl-1H- benzo[d]imidazole-6- carboxylic acid | 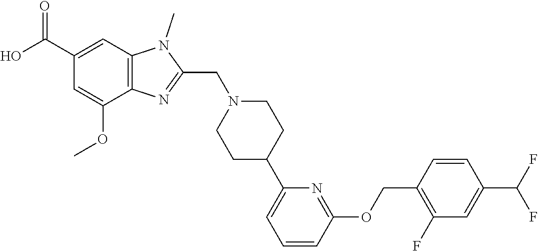 [SMILES: COc1cc(C(=O)O)cc2c1nc(CN1CCC(c3cccc(OCc4ccc(C(F)F)cc4F)n3)CC1)n2C] |
| 306 | 4-Methoxy-2-((4-(6-((2- methoxy-4- (trifluoromethyl)benzyl)oxy) pyridin-2-yl)piperidin-1- yl)methyl)-1-methyl-1H- benzo[d]imidazole-6- carboxylic acid | 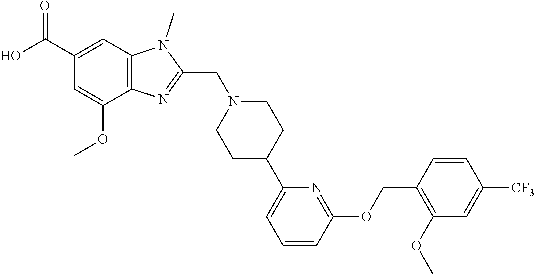 [SMILES: COc1cc(C(F)(F)F)ccc1COc1cccc(C2CCN(Cc3nc4c(OC)cc(C(=O)O)cc4n3C)CC2)n1] |
| 307 | 2-((4-(6-((4-Chloro-2- fluorobenzyl)oxy)pyridin-2- yl)piperidin-1-yl)methyl)-4- hydroxy-1-methyl-1H- benzo[d]imidazole-6- carboxylic acid | 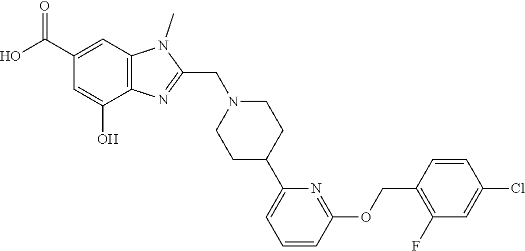 [SMILES: Cn1c(CN2CCC(c3cccc(OCc4ccc(Cl)cc4F)n3)CC2)nc2c(O)cc(C(=O)O)cc21] |
| 308 | 2-((4-(6-((4-Cyano-2- fluorobenzyl)oxy)pyridin-2- yl)piperidin-1-yl)methyl)-4- methoxy-1-methyl-1H- benzo[d]imidazole-6- carboxylic acid | 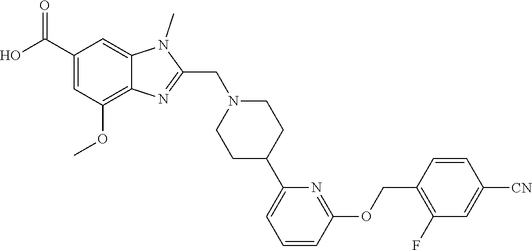 [SMILES: COc1cc(C(=O)O)cc2c1nc(CN1CCC(c3cccc(OCc4ccc(C#N)cc4F)n3)CC1)n2C] |
| 309 | (S)-2-((4-(2-(5- Chloropyridin-2-yl)-2- methylbenzo[d][1,3]dioxol- 4-yl)piperidin-1-yl)methyl)- 4-(2-methoxyethoxy)-1- methyl-1H- benzo[d]imidazole-6- carboxylic acid | 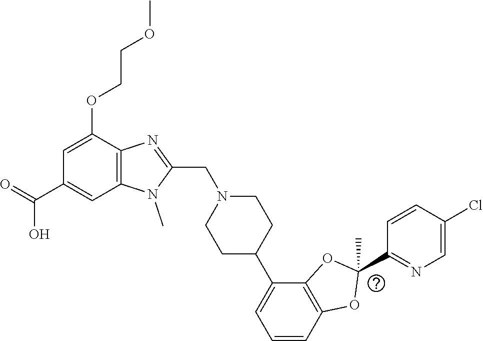 [SMILES: COCCOc1cc(C(=O)O)cc2c1nc(CN1CCC(c3cccc4c3O[C@@](C)(c3ccc(Cl)cn3)O4)CC1)n2C] |
| 310 | (R)-2-((4-(3-(4-cyano-2- fluorophenyl)-2,3- dihydrobenzo[b][1,4]dioxin- 5-yl)piperidin-1-yl)methyl)- 4-methoxy-1-methyl-1H- benzo[d]imidazole-6- carboxylic acid | 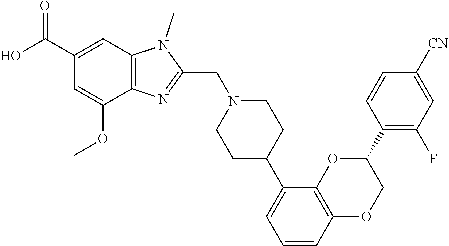 [SMILES: COc1cc(C(=O)O)cc2c1nc(CN1CCC(c3cccc4c3O[C@H](c3ccc(C#N)cc3F)CO4)CC1)n2C] |
| 311 | 2-((4-(6-((4-chloro-2- fluorobenzyl)oxy)pyridin-2- yl)piperidin-1-yl)methyl)-3- methyl-7,8-dihydro-3H- [1,4]dioxino[2′,3′:3,4]benzo [1,2-d]imidazole-5-carboxylic acid | 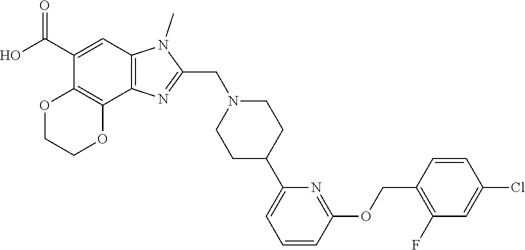 [SMILES: Cn1c(CN2CCC(c3cccc(OCc4ccc(Cl)cc4F)n3)CC2)nc2c3c(c(C(=O)O)cc21)OCCO3] |
| 312 | (R)-2-((4-(6-((4-Cyano-2- fluorobenzyl)oxy)pyridin-2- yl)piperidin-1-yl)methyl)-4- (1-fluoroethoxy)-1-methyl- 1H-benzo[d]imidazole-6- carboxylic acid | 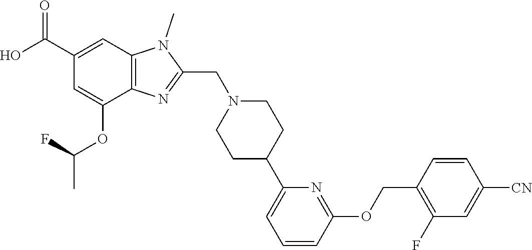 [SMILES: C[C@@H](F)Oc1cc(C(=O)O)cc2c1nc(CN1CCC(c3cccc(OCc4ccc(C#N)cc4F)n3)CC1)n2C] |
| 313 | (S)-2-((4-(6-((4-Cyano-2- fluorobenzyl)oxy)pyridin-2- yl)piperidin-1-yl)methyl)-4- (1-fluoroethoxy)-1-methyl- 1H-benzo[d]imidazole-6- carboxylic acid | 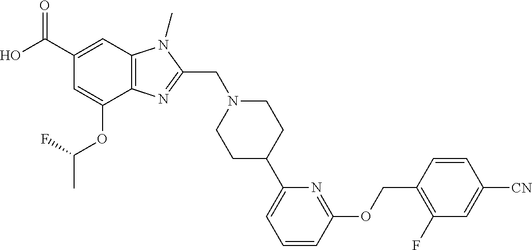 [SMILES: C[C@H](F)Oc1cc(C(=O)O)cc2c1nc(CN1CCC(c3cccc(OCc4ccc(C#N)cc4F)n3)CC1)n2C] |
| 314 | (S)-2-((4-(3-(4-Cyano-2- fluorophenyl)-2,3- dihydrobenzo[b][1,4]dioxin- 5-yl)piperidin-1-yl)methyl)- 4-methoxy-1-methyl-1H- benzo[d]imidazole-6- carboxylic acid | 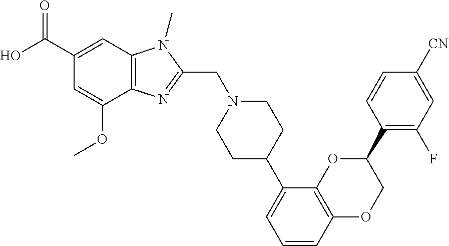 [SMILES: COc1cc(C(=O)O)cc2c1nc(CN1CCC(c3cccc4c3O[C@@H](c3ccc(C#N)cc3F)CO4)CC1)n2C] |
| 315 | (R)-2-((4-(3-(4-Cyano-2- fluorophenyl)-2,3- dihydrobenzo[b][1,4]dioxin- 5-yl)piperidin-1-yl)methyl)- 4-(2-fluoroethoxy)-1-methyl- 1H-benzo[d]imidazole-6- carboxylic acid | 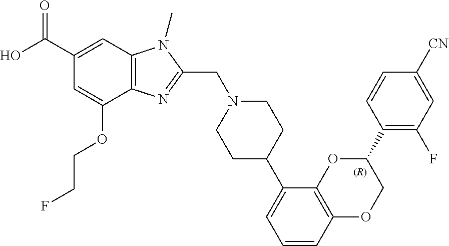 |
| 316 | 5-(2-((4-(6-((4-Chloro-2- fluorobenzyl)oxy)pyridin-2- yl)piperidin-1-yl)methyl)-4- (difluoromethoxy)-1-methyl- 1H-benzo[d]imidazol-6- yl)isoxazol-3-ol | 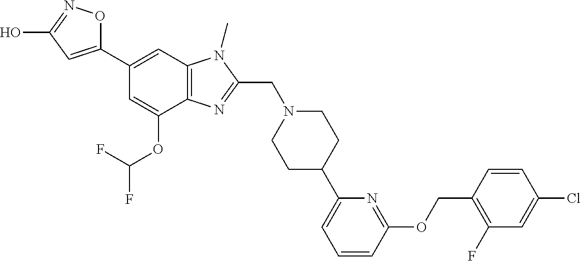 [SMILES: Cn1c(CN2CCC(c3cccc(OCc4ccc(Cl)cc4F)n3)CC2)nc2c(OC(F)F)cc(-c3cc(O)no3)cc21] |
| 317 | 2-((4-(6-((4-Cyano-2- fluorobenzyl)oxy)pyridin-2- yl)piperidin-1-yl)methyl)-4- ((1r,3r)-3- methoxycyclobutoxy)-1- methyl-1H- benzo[d]imidazole-6- carboxylic acid | 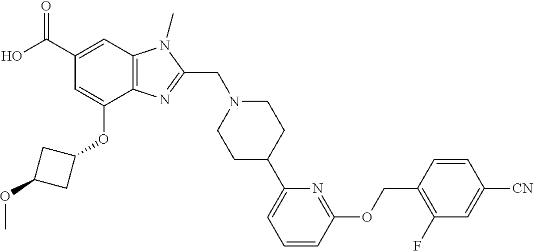 [SMILES: CO[C@H]1C[C@H](Oc2cc(C(=O)O)cc3c2nc(CN2CCC(c4cccc(OCc5ccc(C#N)cc5F)n4)CC2)n3C)C1] |
| 318 | 2-((4-(6-((4-Cyano-2- fluorobenzyl)oxy)pyridin-2- yl)piperidin-1-yl)methyl)-4- ((1s,3s)-3- methoxycyclobutoxy)-1- methyl-1H- benzo[d]imidazole-6- carboxylic acid | 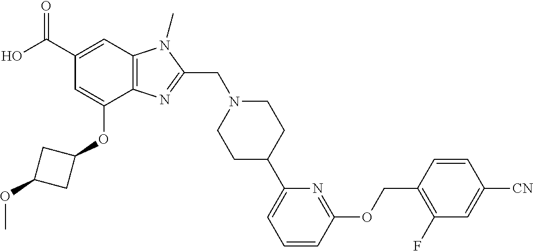 [SMILES: CO[C@H]1C[C@@H](Oc2cc(C(=O)O)cc3c2nc(CN2CCC(c4cccc(OCc5ccc(C#N)cc5F)n4)CC2)n3C)C1] |
| 319 | 2-((4-(6-((4-Cyano-2- fluorobenzyl)oxy)pyridin-2- yl)piperidin-1-yl)methyl)-1- methyl-4-propoxy-1H- benzo[d]imidazole-6- carboxylic acid | 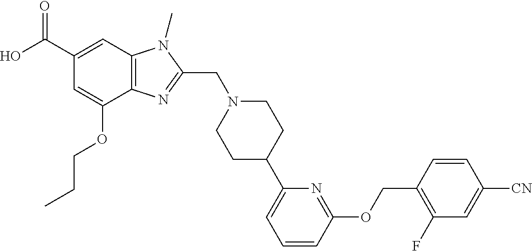 [SMILES: CCCOc1cc(C(=O)O)cc2c1nc(CN1CCC(c3cccc(OCc4ccc(C#N)cc4F)n3)CC1)n2C] |
| 320 | 2-((4-(6-((4-Chloro-2- fluorobenzyl)oxy)pyridin-2- yl)piperidin-1-yl)methyl)-4- ((1-hydroxypropan-2- yl)oxy)-1-methyl-1H- benzo[d]imidazole-6- carboxylic acid | 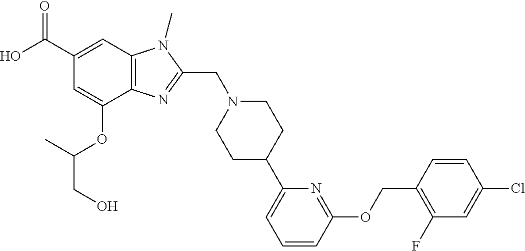 [SMILES: CC(CO)Oc1cc(C(=O)O)cc2c1nc(CN1CCC(c3cccc(OCc4ccc(Cl)cc4F)n3)CC1)n2C] |
| 321 | 2-((4-(6-((4-Chloro-2- fluorophenyl)methoxy- d2)pyridin-2-yl)piperidin-1- yl)methyl-d2)-4- (difluoromethoxy)-1-methyl- 1H-benzo[d]imidazole-6- carboxylic acid | 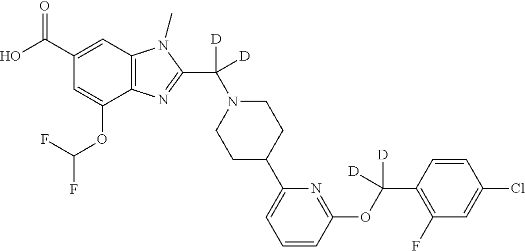 [SMILES: [2H]C([2H])(Oc1cccc(C2CCN(C([2H])([2H])c3nc4c(OC(F)F)cc(C(=O)O)cc4n3C)CC2)n1)c1ccc(Cl)cc1F] |
| 322 | 2-((4-(6-((4-chloro-2- fluorobenzyl)oxy)pyridin-2- yl)piperidin-1-yl)methyl)-1- methyl-4-propoxy-1H- benzo[d]imidazole-6- carboxylic acid | 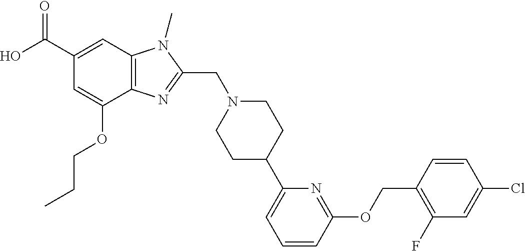 [SMILES: CCCOc1cc(C(=O)O)cc2c1nc(CN1CCC(c3cccc(OCc4ccc(Cl)cc4F)n3)CC1)n2C] |
| 323 | 2-((4-(6-((4-Cyano-2- fluorobenzyl)oxy)pyridin-2- yl)piperidin-1-yl)methyl)-7- methoxy-3-methyl-3H- imidazo[4,5-b]pyridine-5- carboxylic acid | 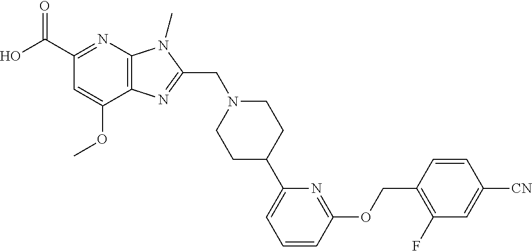 [SMILES: COc1cc(C(=O)O)nc2c1nc(CN1CCC(c3cccc(OCc4ccc(C#N)cc4F)n3)CC1)n2C] |
| 324 | 2-((4-(6-((4-Chloro-2- fluorobenzyl)oxy)pyridin-2- yl)piperidin-1-yl)methyl)-7- methoxy-3-methyl-3H- imidazo[4,5-b]pyridine-5- carboxylic acid | 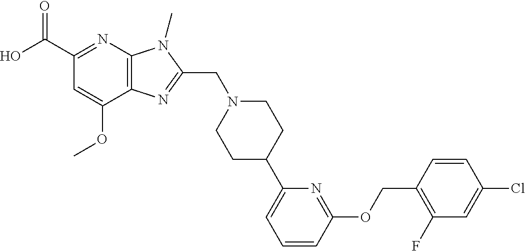 [SMILES: COc1cc(C(=O)O)nc2c1nc(CN1CCC(c3cccc(OCc4ccc(Cl)cc4F)n3)CC1)n2C] |
| 325 | 2-((4-(6-((4-Chloro-2- fluorobenzyl)oxy)pyridin-2- yl)piperidin-1-yl)methyl)-4- ((1r,3r)-3- methoxycyclobutoxy)-1- methyl-1H- benzo[d]imidazole-6- carboxylic acid | 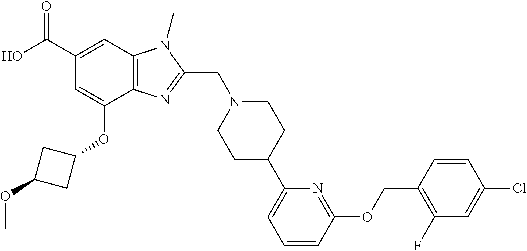 [SMILES: CO[C@H]1C[C@H](Oc2cc(C(=O)O)cc3c2nc(CN2CCC(c4cccc(OCc5ccc(Cl)cc5F)n4)CC2)n3C)C1] |
| 326 | (S)-2-((4-(2-(4-Cyano-2- fluorophenyl)-2,3- dihydrobenzo[b][1,4]dioxin- 5-yl)piperidin-1-yl)methyl)- 4-(difluoromethoxy)-1- methyl-1H- benzo[d]imidazole-6- carboxylic acid | 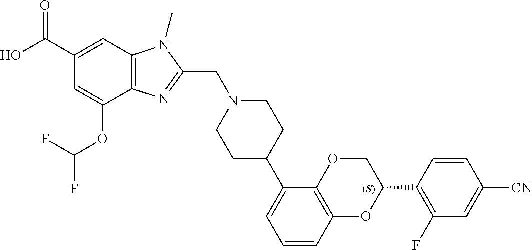 |
| 327 | 2-((4-(6-((4-Cyano-2,5- difluorobenzyl)oxy)pyridin- 2-yl)piperidin-1-yl)methyl)- 4-methoxy-1-methyl-1H- benzo[d]imidazole-6- carboxylic acid | 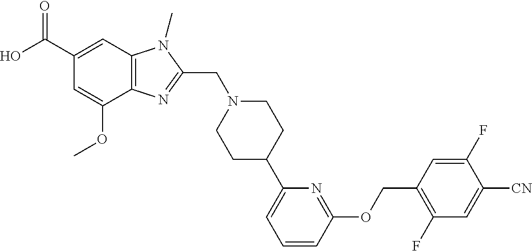 [SMILES: COc1cc(C(=O)O)cc2c1nc(CN1CCC(c3cccc(OCc4cc(F)c(C#N)cc4F)n3)CC1)n2C] |
| 328 | 2-((4-(6-((4-Chloro-2- fluorobenzyl)oxy)pyridin-2- yl)piperidin-1-yl)methyl)-7- (difluoromethoxy)-3-methyl- 3H-imidazo[4,5-b]pyridine- 5-carboxylic acid | 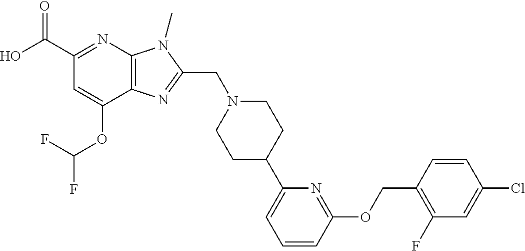 [SMILES: Cn1c(CN2CCC(c3cccc(OCc4ccc(Cl)cc4F)n3)CC2)nc2c(OC(F)F)cc(C(=O)O)nc21] |
| 329 | (S)-2-((4-(6-((4-chloro-2- fluorobenzyl)oxy)pyridin-2- yl)piperidin-1-yl)methyl)-4- (1,2-difluoroethoxy)-1- methyl-1H- benzo[d]imidazole-6- carboxylic acid | 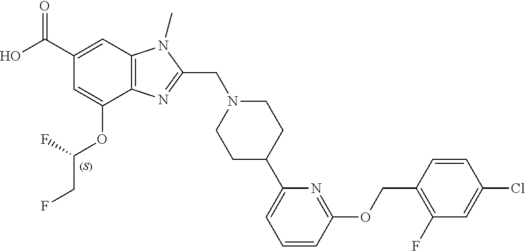 |
| 330 | (R)-2-((4-(6-((4-chloro-2- fluorobenzyl)oxy)pyridin-2- yl)piperidin-1-yl)methyl)-4- (1,2-difluoroethoxy)-1- methyl-1H- benzo[d]imidazole-6- carboxylic acid | 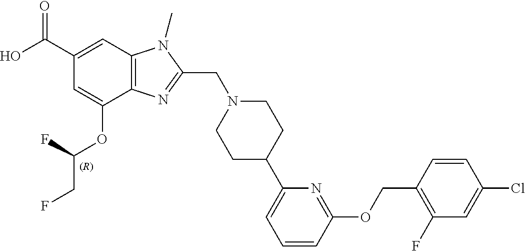 |
| 331 | 2-((4-(6-((4-Chloro-2,3- dihydrobenzofuran-7- yl)methoxy)pyridin-2- yl)piperidin-1-yl)methyl)-4- (difluoromethoxy)-1-methyl- 1H-benzo[d]imidazole-6- carboxylic acid | 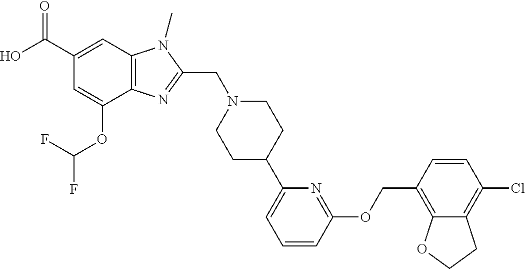 [SMILES: Cn1c(CN2CCC(c3cccc(OCc4ccc(Cl)c5c4OCC5)n3)CC2)nc2c(OC(F)F)cc(C(=O)O)cc21] |
| 332 | (S)-2-((4-(3-(4-chloro-2- fluorophenyl)-2,3- dihydrobenzo[b][1,4]dioxin- 5-yl)piperidin-1-yl)methyl)- 4-(2-fluoroethoxy)-1-methyl- 1H-benzo[d]imidazole-6- carboxylic acid | 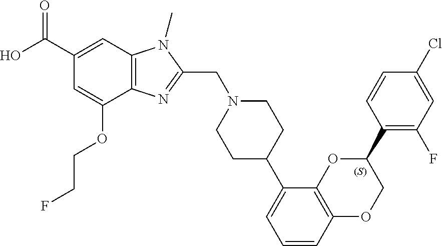 |
| 333 | (R)-2-((4-(3-(4-chloro-2- fluorophenyl)-2,3- dihydrobenzo[b][1,4]dioxin- 5-yl)piperidin-1-yl)methyl)- 4-(2-fluoroethoxy)-1-methyl- 1H-benzo[d]imidazole-6- carboxylic acid | 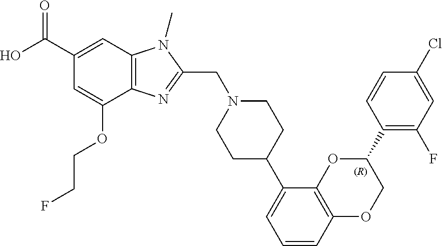 |
| 334 | (S)-2-((4-(3-(4-Chloro-2- fluorophenyl)-2,3- dihydrobenzo[b][1,4]dioxin- 5-yl)piperidin-1-yl)methyl)- 4-(difluoromethoxy)-1- methyl-1H- benzo[d]imidazole-6- carboxylic acid | 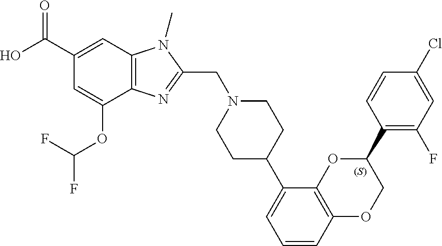 |
| 335 | (R)-2-((4-(3-(4-Chloro-2- fluorophenyl)-2,3- dihydrobenzo[b][1,4]dioxin- 5-yl)piperidin-1-yl)methyl)- 4-(difluoromethoxy)-1- methyl-1H- benzo[d]imidazole-6- carboxylic acid | 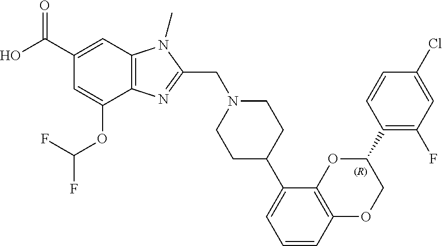 |
| 336 | 2-((4-(6-((4-Cyano-2- fluorobenzyl)oxy)pyridin-2- yl)piperidin-1-yl)methyl)-6- fluoro-7-methoxy-1-methyl- 1H-benzo[d]imidazole-5- carboxylic acid | 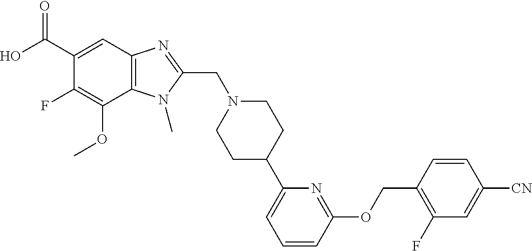 [SMILES: COc1c(F)c(C(=O)O)cc2nc(CN3CCC(c4cccc(OCc5ccc(C#N)cc5F)n4)CC3)n(C)c12] |
| 337 | 2-((4-(6-((4-Chloro-2- fluorobenzyl)oxy)pyridin-2- yl)piperidin-1-yl)methyl)-5- fluoro-4-methoxy-1-methyl- 1H-benzo[d]imidazole-6- carboxylic acid | 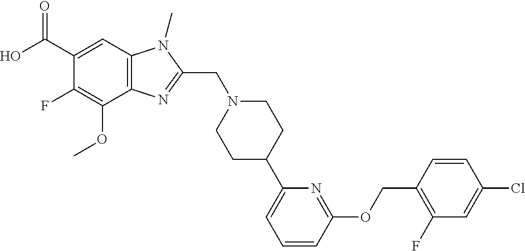 [SMILES: COc1c(F)c(C(=O)O)cc2c1nc(CN1CCC(c3cccc(OCc4ccc(Cl)cc4F)n3)CC1)n2C] |
| 338 | 2-((4-(6-((4-chloro-2- fluorobenzyl)oxy)pyridin-2- yl)piperidin-1-yl)methyl)-6- fluoro-7-methoxy-1-methyl- 1H-benzo[d]imidazole-5- carboxylic acid | 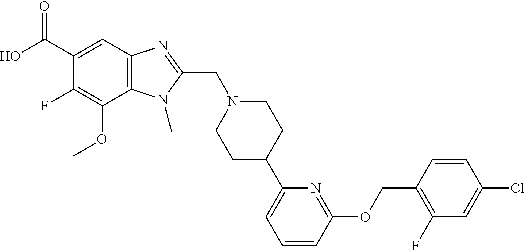 [SMILES: COc1c(F)c(C(=O)O)cc2nc(CN3CCC(c4cccc(OCc5ccc(Cl)cc5F)n4)CC3)n(C)c12] |
| 339 | 2-((4-(3-((4-Cyano-2- fluorobenzyl)oxy)-4- fluorophenyl)piperidin-1- yl)methyl)-7-fluoro-4- methoxy-1-methyl-1H- benzo[d]imidazole-6- carboxylic acid | 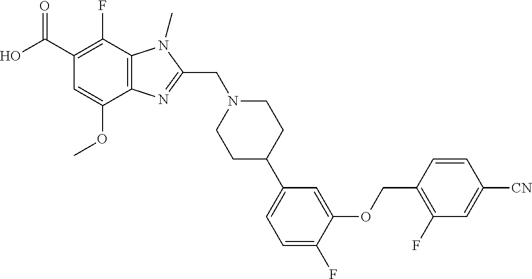 [SMILES: COc1cc(C(=O)O)c(F)c2c1nc(CN1CCC(c3ccc(F)c(OCc4ccc(C#N)cc4F)c3)CC1)n2C] |
| 340 | 2-((4-(3-((4-Chloro-2- fluorobenzyl)oxy)-4- fluorophenyl)piperidin-1- yl)methyl)-7-fluoro-4- methoxy-1-methyl-1H- benzo[d]imidazole-6- carboxylic acid | 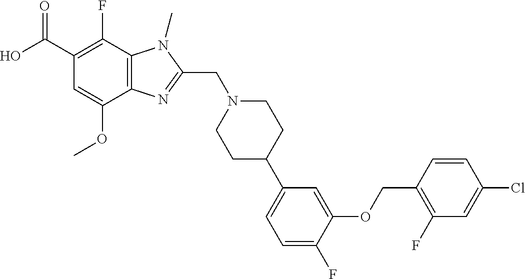 [SMILES: COc1cc(C(=O)O)c(F)c2c1nc(CN1CCC(c3ccc(F)c(OCc4ccc(Cl)cc4F)c3)CC1)n2C] |
| 341 | 2-((4-(6-((4-Chloro-2- fluorobenzyl)oxy)pyridin-2- yl)piperidin-1-yl)methyl)-7- hydroxy-1-methyl-1H- benzo[d]imidazole-6- carboxylic acid | 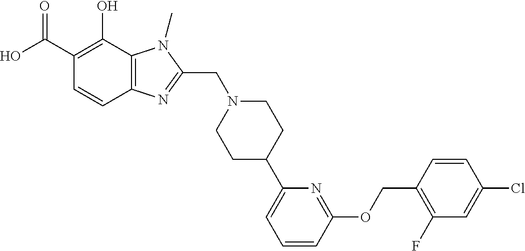 [SMILES: Cn1c(CN2CCC(c3cccc(OCc4ccc(Cl)cc4F)n3)CC2)nc2ccc(C(=O)O)c(O)c21] |
| 342 | 2-(4-(6-((4-cyano-2- fluorobenzyl)oxy)pyridin-2- yl)-2-fluorobenzyl)-4- methoxy-1-methyl-1H- benzo[d]imidazole-6- carboxylic acid | 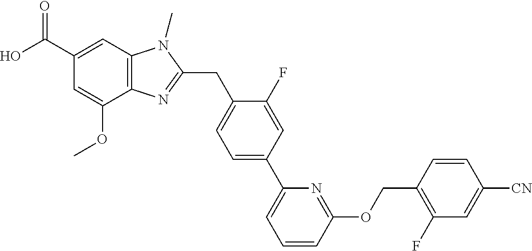 [SMILES: COc1cc(C(=O)O)cc2c1nc(Cc1ccc(-c3cccc(OCc4ccc(C#N)cc4F)n3)cc1F)n2C] |
| 343 | 2-((4-(6-((4-Chloro-2- fluorobenzyl)oxy)pyridin-2- yl)piperidin-1-yl)methyl)-4- (2-hydroxyethoxy)-1-methyl- 1H-benzo[d]imidazole-6- carboxylic acid | 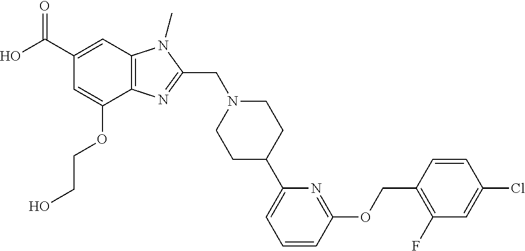 [SMILES: Cn1c(CN2CCC(c3cccc(OCc4ccc(Cl)cc4F)n3)CC2)nc2c(OCCO)cc(C(=O)O)cc21] |
| 344 | 2-((4-(6-((4-Cyano-2- methoxybenzyl)oxy)pyridin- 2-yl)piperidin-1-yl)methyl)- 4-methoxy-1-methyl-1H- benzo[d]imidazole-6- carboxylic acid | 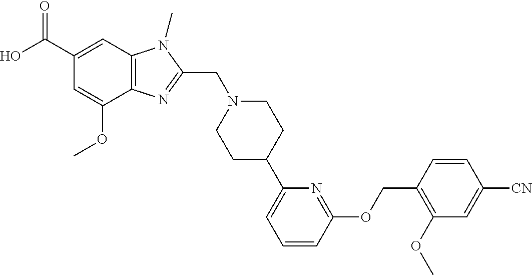 [SMILES: COc1cc(C#N)ccc1COc1cccc(C2CCN(Cc3nc4c(OC)cc(C(=O)O)cc4n3C)CC2)n1] |
| 345 | 2-((4-(6-((4-Chloro-2- fluorobenzyl)oxy)pyridin-2- yl)piperidin-1-yl)methyl)-4- hydroxy-1-methyl-1H- benzo[d]imidazole-5- carboxylic acid | 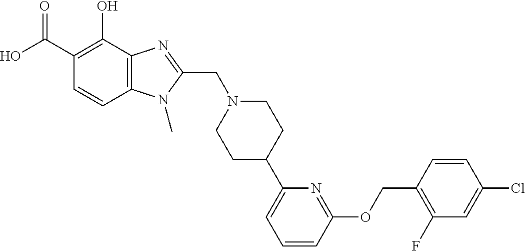 [SMILES: Cn1c(CN2CCC(c3cccc(OCc4ccc(Cl)cc4F)n3)CC2)nc2c(O)c(C(=O)O)ccc21] |
| 346 | 2-((4-(6-((4-Chloro-2- fluorobenzyl)oxy)pyridin-2- yl)piperidin-1-yl)methyl)-4- (fluoromethoxy)-1- (fluoromethyl)-1H- benzo[d]imidazole-6- carboxylic acid | 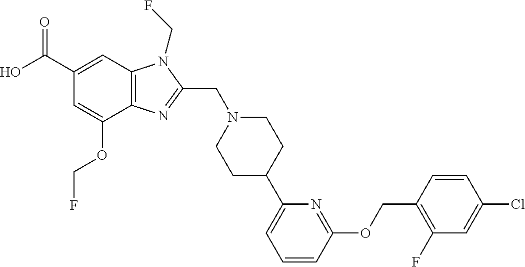 [SMILES: O=C(O)c1cc(OCF)c2nc(CN3CCC(c4cccc(OCc5ccc(Cl)cc5F)n4)CC3)n(CF)c2c1] |
| 347 | 2-((4-(6-((4-Chloro-2- fluorobenzyl)oxy)pyridin-2- yl)piperidin-1-yl)methyl)-7- (fluoromethoxy)-1- (fluoromethyl)-1H- benzo[d]imidazole-5- carboxylic acid | 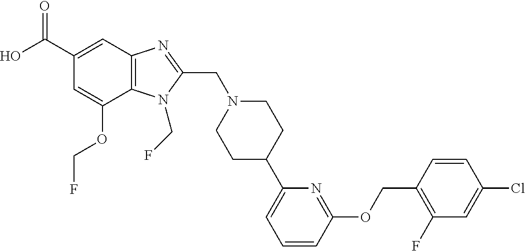 [SMILES: O=C(O)c1cc(OCF)c2c(c1)nc(CN1CCC(c3cccc(OCc4ccc(Cl)cc4F)n3)CC1)n2CF] |
| 348 | 2-((4-(6-((4-Chloro-2- methoxybenzyl)oxy)pyridin- 2-yl)piperidin-1-yl)methyl)- 4-methoxy-1-methyl-1H- benzo[d]imidazole-6- carboxylic acid | 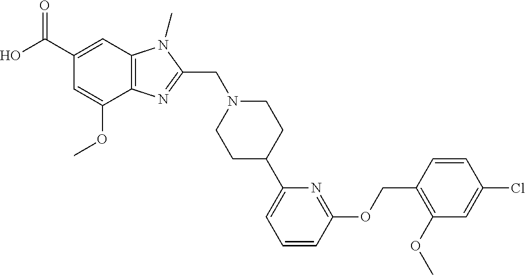 [SMILES: COc1cc(Cl)ccc1COc1cccc(C2CCN(Cc3nc4c(OC)cc(C(=O)O)cc4n3C)CC2)n1] |
| 349 | 2-((4-(6-((4-Cyano-2- fluorobenzyl)oxy)pyridin-2- yl)piperidin-1-yl)methyl)-4- (methoxy-d3)-1-methyl-1H- benzo[d]imidazole-6- carboxylic acid | 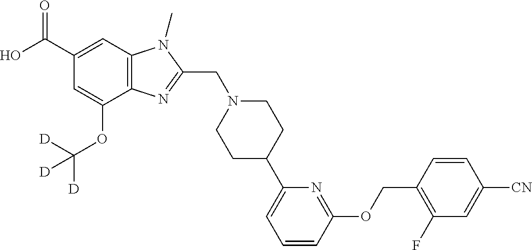 [SMILES: [2H]C([2H])([2H])Oc1cc(C(=O)O)cc2c1nc(CN1CCC(c3cccc(OCc4ccc(C#N)cc4F)n3)CC1)n2C] |
| 350 | 2-((4-(6-((6-Cyano-4- fluoropyridin-3- yl)methoxy)pyridin-2- yl)piperidin-1-yl)methyl)-4- (difluoromethoxy)-1-methyl- 1H-benzo[d]imidazole-6- carboxylic acid | 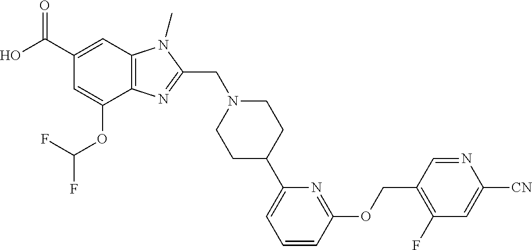 [SMILES: Cn1c(CN2CCC(c3cccc(OCc4cnc(C#N)cc4F)n3)CC2)nc2c(OC(F)F)cc(C(=O)O)cc21] |
| 351 | 2-((4-(6-((4-Chloro-2- fluorobenzyl)oxy)pyridin-2- yl)piperidin-1-yl)methyl)-4- (1-fluoropropoxy)-1-methyl- 1H-benzo[d]imidazole-6- carboxylic acid | 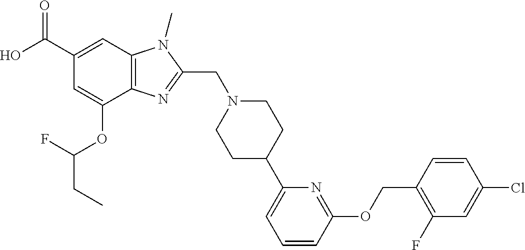 [SMILES: CCC(F)Oc1cc(C(=O)O)cc2c1nc(CN1CCC(c3cccc(OCc4ccc(Cl)cc4F)n3)CC1)n2C] |
| 352 | 2-((4-(6-((4-Cyano-2- fluorobenzyl)oxy)pyridin-2- yl)piperidin-1-yl)methyl)-4- (4-fluorophenoxy)-1-methyl- 1H-benzo[d]imidazole-6- carboxylic acid | 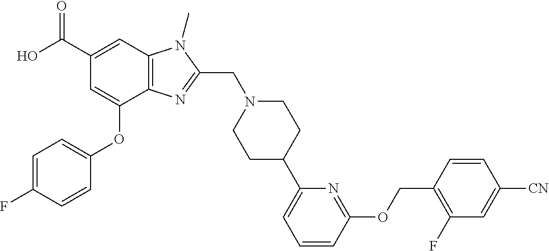 [SMILES: Cn1c(CN2CCC(c3cccc(OCc4ccc(C#N)cc4F)n3)CC2)nc2c(Oc3ccc(F)cc3)cc(C(=O)O)cc21] |
| 353 | 2-((4-(6-((4-Chloro-2- fluorobenzyl)oxy)pyridin-2- yl)piperidin-1-yl)methyl)-4- ((1r,3r)-3- fluorocyclobutoxy)-1- methyl-1H- benzo[d]imidazole-6- carboxylic acid | 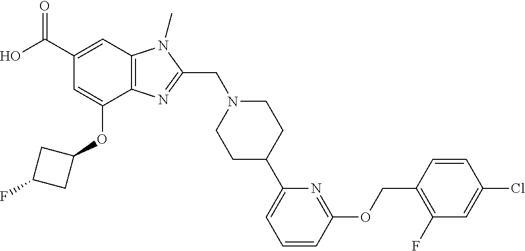 [SMILES: Cn1c(CN2CCC(c3cccc(OCc4ccc(Cl)cc4F)n3)CC2)nc2c(O[C@H]3C[C@H](F)C3)cc(C(=O)O)cc21] |
| 354 | 2-((4-(2-((4-Chloro-2- fluorobenzyl)oxy)-5- fluoropyrimidin-4- yl)piperidin-1-yl)methyl)-4- (difluoromethoxy)-1-methyl- 1H-benzo[d]imidazole-6- carboxylic acid | 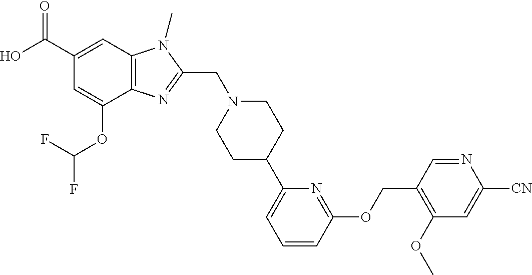 [SMILES: COc1cc(C#N)ncc1COc1cccc(C2CCN(Cc3nc4c(OC(F)F)cc(C(=O)O)cc4n3C)CC2)n1] |
| 355 | 2-((4-(3-((4-Chloro-2- fluorobenzyl)oxy)pyridazin- 4-yl)piperidin-1-yl)methyl)- 4-(difluoromethoxy)-1- methyl-1H- benzo[d]imidazole-6- carboxylic acid | 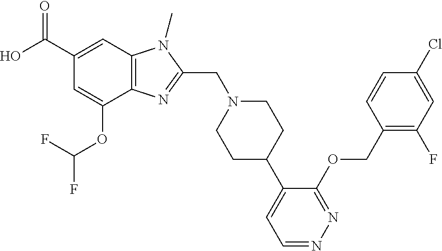 [SMILES: Cn1c(CN2CCC(c3ccnnc3OCc3ccc(Cl)cc3F)CC2)nc2c(OC(F)F)cc(C(=O)O)cc21] |
| 356 | 2-((4-(4-((4-Chloro-2- fluorobenzyl)oxy)-5- fluoropyrimidin-2- yl)piperidin-1-yl)methyl)-4- (difluoromethoxy)-1-methyl- 1H-benzo[d]imidazole-6- carboxylic acid | 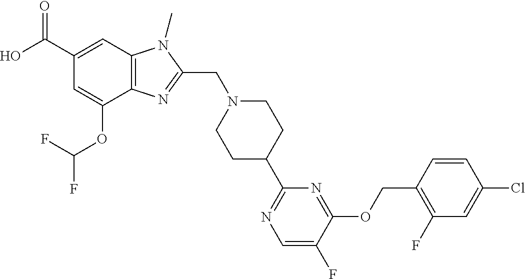 [SMILES: Cn1c(CN2CCC(c3ncc(F)c(OCc4ccc(Cl)cc4F)n3)CC2)nc2c(OC(F)F)cc(C(=O)O)cc21] |
| 357 | 2-((4-(6-((4-Chloro-2- fluorobenzyl)oxy)pyridin-2- yl)piperidin-1-yl)methyl)-4- (cyclopropylmethoxy)-1- methyl-1H- benzo[d]imidazole-6- carboxylic acid | 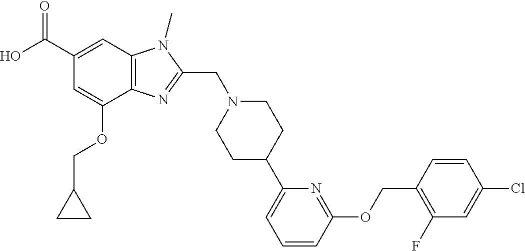 [SMILES: Cn1c(CN2CCC(c3cccc(OCc4ccc(Cl)cc4F)n3)CC2)nc2c(OCC3CC3)cc(C(=O)O)cc21] |
| 358 | 2-((4-(6-((4-Chloro-2- fluorobenzyl)oxy)pyridin-2- yl)piperidin-1-yl)methyl)-4- ethoxy-1-methyl-1H- benzo[d]imidazole-6- carboxylic acid | 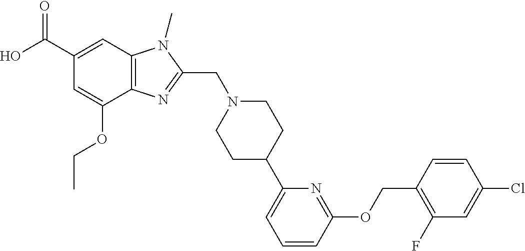 [SMILES: CCOc1cc(C(=O)O)cc2c1nc(CN1CCC(c3cccc(OCc4ccc(Cl)cc4F)n3)CC1)n2C] |
| 359 | -((4-(6-((4-Chloro-2- fluorobenzyl)oxy)pyridin-2- yl)piperidin-1-yl)methyl)-4- (1-fluoroethoxy)-1-methyl- 1H-benzo[d]imidazole-6- carboxylic acid | 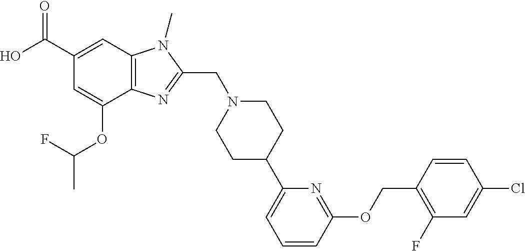 [SMILES: CC(F)Oc1cc(C(=O)O)cc2c1nc(CN1CCC(c3cccc(OCc4ccc(Cl)cc4F)n3)CC1)n2C] |
| 360 | (S)-2-((4-(6-((4-chloro-2- fluorobenzyl)oxy)pyridin-2- yl)piperidin-1-yl)methyl)-4- (1-fluoroethoxy)-1-methyl- 1H-benzo[d]imidazole-6- carboxylic acid | 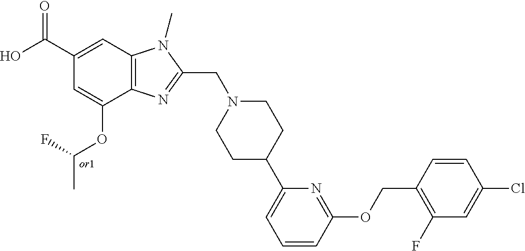 [SMILES: C[C@H](F)Oc1cc(C(=O)O)cc2c1nc(CN1CCC(c3cccc(OCc4ccc(Cl)cc4F)n3)CC1)n2C] |
| 361 | (R)-2-((4-(6-((4-Chloro-2- fluorobenzyl)oxy)pyridin-2- yl)piperidin-1-yl)methyl)-4- (1-fluoroethoxy)-1-methyl- 1H-benzo[d]imidazole-6- carboxylic acid | 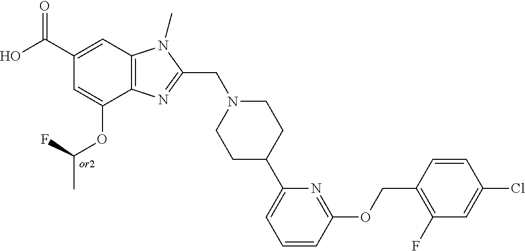 [SMILES: C[C@@H](F)Oc1cc(C(=O)O)cc2c1nc(CN1CCC(c3cccc(OCc4ccc(Cl)cc4F)n3)CC1)n2C] |
| 362 | 2-((3-(6-((4-Chloro-2- fluorophenoxy)methyl) pyridin-2-yl)pyrrolidin-1- yl)methyl)-4- (difluoromethoxy)-1-methyl- 1H-benzo[d]imidazole-6- carboxylic acid | 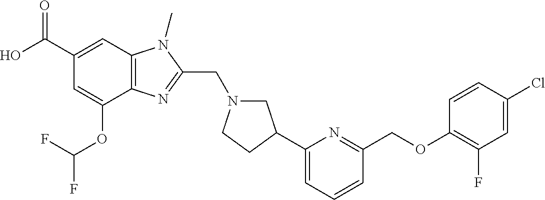 [SMILES: Cn1c(CN2CCC(c3cccc(COc4ccc(Cl)cc4F)n3)C2)nc2c(OC(F)F)cc(C(=O)O)cc21] |
| 363 | 2-(((1r,4r)-4-(6-((4-Chloro- 2-fluorobenzyl)oxy)pyridin- 2-yl)cyclohexyl) (methyl)amino)- 4-methoxy-1-methyl-1H- benzo[d]imidazole-6- carboxylic acid | 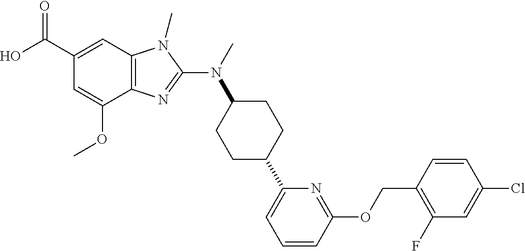 |
| 364 | 2-((4-(2-((4-Chloro-2- fluorobenzyl)oxy)pyridin-3- yl)piperidin-1-yl)methyl)-4- (difluoromethoxy)-1-methyl- 1H-benzo[d]imidazole-6- carboxylic acid | 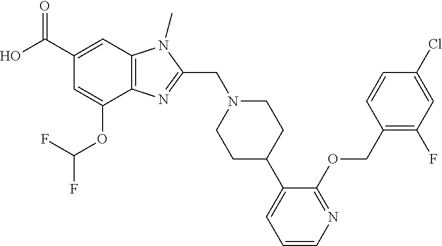 [SMILES: Cn1c(CN2CCC(c3cccnc3OCc3ccc(Cl)cc3F)CC2)nc2c(OC(F)F)cc(C(=O)O)cc21] |
| 365 | 2-((4-(6-((4-Cyclopropyl-2- fluorobenzyl)oxy)pyridin-2- yl)piperidin-1-yl)methyl)-4- (difluoromethoxy)-1-methyl- 1H-benzo[d]imidazole-6- carboxylic acid | 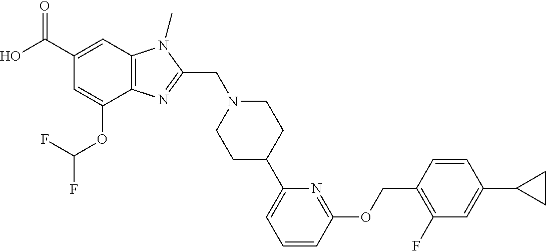 [SMILES: Cn1c(CN2CCC(c3cccc(OCc4ccc(C5CC5)cc4F)n3)CC2)nc2c(OC(F)F)cc(C(=O)O)cc21] |
| 366 | 2-((4-(6-((4-Cyano-2,5- difluorobenzyl)oxy)pyridin- 2-yl)piperidin-1-yl)methyl)- 4-(difluoromethoxy)-1- methyl-1H- benzo[d]imidazole-6- carboxylic acid | 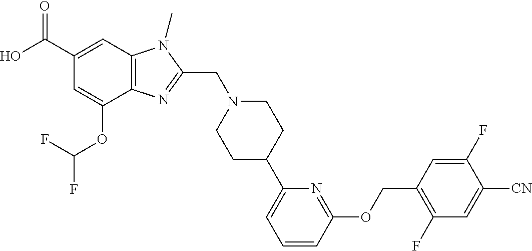 [SMILES: Cn1c(CN2CCC(c3cccc(OCc4cc(F)c(C#N)cc4F)n3)CC2)nc2c(OC(F)F)cc(C(=O)O)cc21] |
| 367 | 2-(4-((6-((4-chloro-2- fluorophenoxy)methyl) pyridin-2-yl) oxy)-2-fluorobenzyl)- 4-(difluoromethoxy)-1- methyl-1H- benzo[d]imidazole-6- carboxylic acid | 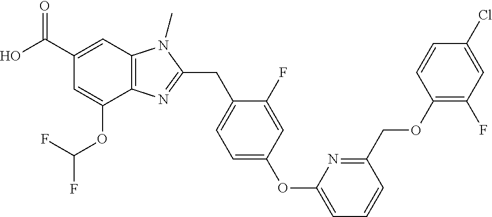 [SMILES: Cn1c(Cc2ccc(Oc3cccc(COc4ccc(Cl)cc4F)n3)cc2F)nc2c(OC(F)F)cc(C(=O)O)cc21] |
| 368 | S)-2-((4-(2-(4-Chloro-2- fluorophenyl)chroman-8- yl)piperidin-1-yl)methyl)-4- (difluoromethoxy)-1-methyl- 1H-benzo[d]imidazole-6- carboxylic acid | 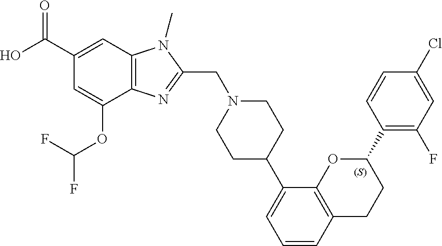 |
| 369 | (R)-2-((4-(2-(4-Chloro-2- fluorophenyl)chroman-8- yl)piperidin-1-yl)methyl)-4- (difluoromethoxy)-1-methyl- 1H-benzo[d]imidazole-6- carboxylic acid | 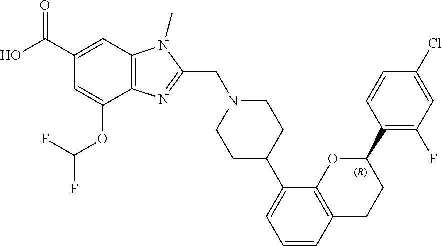 |
| 370 | 3-(2-((4-(6-((4-Chloro-2- fluorobenzyl)oxy)pyridin-2- yl)piperidin-1-yl)methyl)-4- (difluoromethoxy)-1-methyl- 1H-benzo[d]imidazol-6-yl)- 1,2,4-thiadiazol-5(4H)-one | 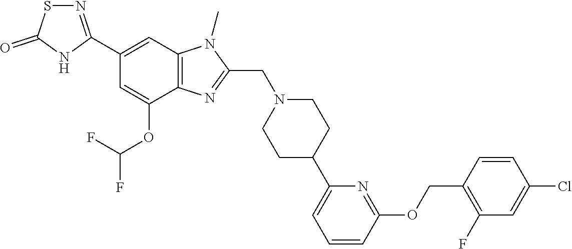 [SMILES: Cn1c(CN2CCC(c3cccc(OCc4ccc(Cl)cc4F)n3)CC2)nc2c(OC(F)F)cc(-c3nsc(=O)[nH]3)cc21] |
| 371 | 2-(((1r,4r)-4-(6-((4-Chloro- 2-fluorobenzyl)oxy)pyridin- 2-yl)cyclohexyl)amino)-4- (difluoromethoxy)-1-methyl- 1H-benzo[d]imidazole-6- carboxylic acid | 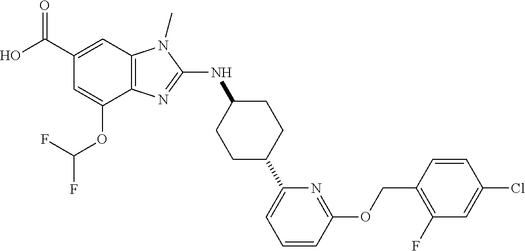 |
| 372 | 2-(((1r,4r)-4-(6-((4-Chloro- 2- fluorobenzyl)o186yridinedin- 2-yl)cyclohexyl)(methyl- d3)amino)-4- (difluoromethoxy)-1-methyl- 1H-benzo[d]imidazole-6- carboxylic acid | 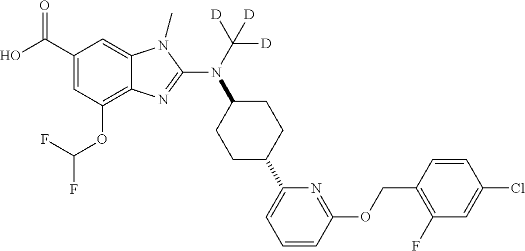 |
| 373 | 2-((4-(4-(4-Chloro-2- fluorobenzyl)-3,4-dihydro- 2H-benzo[b][1,4]oxazin-6- yl)piperidin-1-yl)methyl)-4- (difluoromethoxy)-1-methyl- 1H-benzo[d]imidazole-6- carboxylic acid | 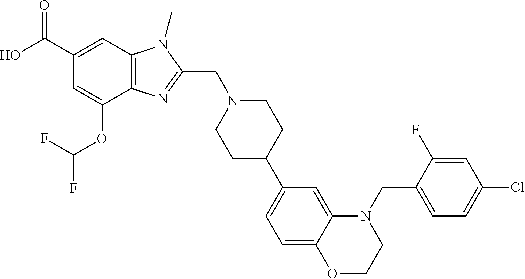 [SMILES: Cn1c(CN2CCC(c3ccc4c(c3)N(Cc3ccc(Cl)cc3F)CCO4)CC2)nc2c(OC(F)F)cc(C(=O)O)cc21] |
| 374 | 2-((4-(6-((4-Chloro-2- fluorobenzyl)oxy)pyrazin-2- yl)piperidin-1-yl)methyl)-4- (difluoromethoxy)-1-methyl- 1H-benzo[d]imidazole-6- carboxylic acid | 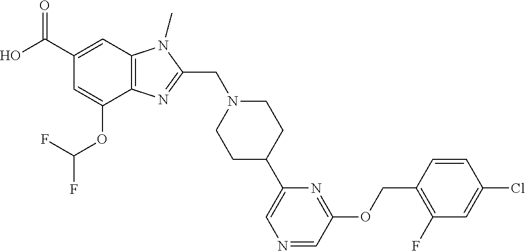 [SMILES: Cn1c(CN2CCC(c3cncc(OCc4ccc(Cl)cc4F)n3)CC2)nc2c(OC(F)F)cc(C(=O)O)cc21] |
| 375 | 2-((4-(2-((4-Chloro-2- fluorobenzyl)oxy)-5- fluoropyrimidin-4- yl)piperidin-1-yl)methyl)-4- (difluoromethoxy)-1-methyl- 1H-benzo[d]imidazole-6- carboxylic acid | 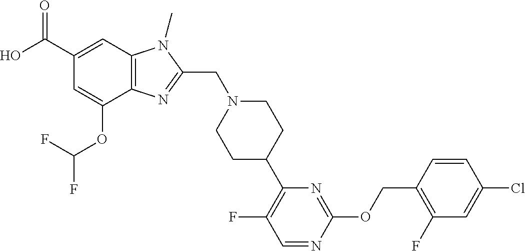 [SMILES: Cn1c(CN2CCC(c3nc(OCc4ccc(Cl)cc4F)ncc3F)CC2)nc2c(OC(F)F)cc(C(=O)O)cc21] |
| 376 | (S)-2-((4-(6-((4-Cyano-2- fluorobenzyl)oxy)pyridin-2- yl)piperidin-1-yl)methyl)-4- hydroxy-1-(oxetan-2- ylmethyl)-1H- benzo[d]imidazole-5- carboxylic acid | 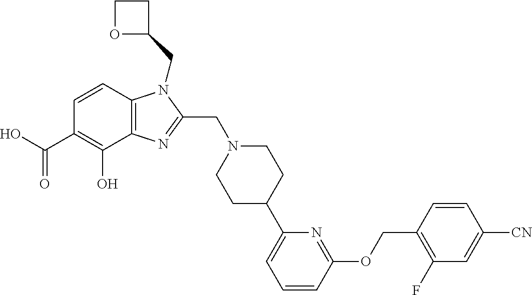 [SMILES: N#Cc1ccc(COc2cccc(C3CCN(Cc4nc5c(O)c(C(=O)O)ccc5n4C[C@@H]4CCO4)CC3)n2)c(F)c1] |
| 377 | (S)-2-((4-(6-((4-Cyano-2- fluorobenzyl)oxy)pyridin-2- yl)piperidin-1-yl)methyl)-7- hydroxy-1-(oxetan-2- ylmethyl)-1H- benzo[d]imidazole-6- carboxylic acid | 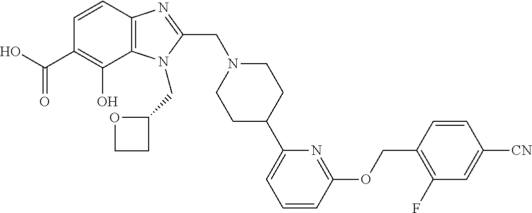 [SMILES: N#Cc1ccc(COc2cccc(C3CCN(Cc4nc5ccc(C(=O)O)c(O)c5n4C[C@@H]4CCO4)CC3)n2)c(F)c1] |
| 378 | (S)-2-((4-(6-((4-Cyano-2- fluorobenzyl)oxy)pyridin-2- yl)piperidin-1-yl)methyl)-1- (oxetan-2-ylmethyl)-1H- imidazo[4,5-c]pyridine-6- carboxylic acid | 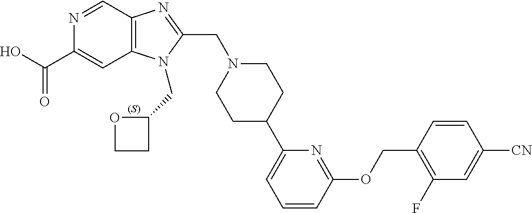 |
| 379 | 2-((4-(6-((4-Chloro-2- fluorobenzyl)oxy)pyridin-2- yl)piperidin-1-yl)methyl)-4- ((1s,3s)-3- fluorocyclobutoxy)-1- methyl-1H- benzo[d]imidazole-6- carboxylic acid | 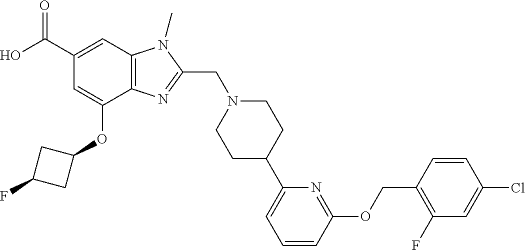 [SMILES: Cn1c(CN2CCC(c3cccc(OCc4ccc(Cl)cc4F)n3)CC2)nc2c(O[C@H]3C[C@@H](F)C3)cc(C(=O)O)cc21] |
| 380 | 2-((4-(6-((4-Chloro-2- fluorobenzyl)oxy)pyridin-2- yl)piperidin-1-yl)methyl)-4- methoxy-1-methyl-1H- imidazo[4,5-c]pyridine-6- carboxylic acid | 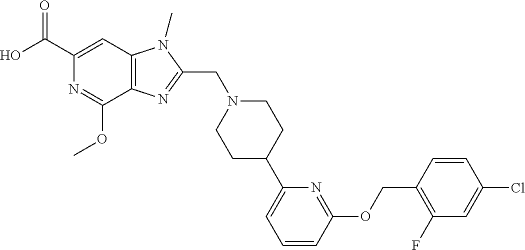 [SMILES: COc1nc(C(=O)O)cc2c1nc(CN1CCC(c3cccc(OCc4ccc(Cl)cc4F)n3)CC1)n2C] |
| 381 | 2-((4-(6-((4-Chloro-2- fluorophenyl)methoxy- d2)pyridin-2-yl)piperidin-1- yl)methyl-d2)-4-(ethoxy-1,1- d2)-1-methyl-1H- benzo[d]imidazole-6- carboxylic acid | 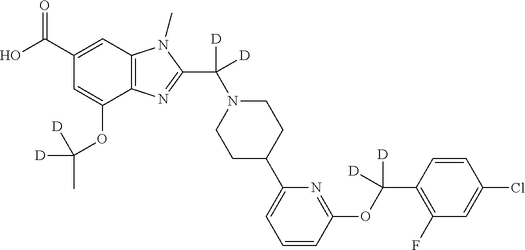 [SMILES: [2H]C([2H])(C)Oc1cc(C(=O)O)cc2c1nc(C([2H])([2H])N1CCC(c3cccc(OC([2H])([2H])c4ccc(Cl)cc4F)n3)CC1)n2C] |
| 382 | (S)-5-(2-((4-(6-((4-Chloro-2- fluorobenzyl)oxy)pyridin-2- yl)piperidin-1-yl)methyl)-4- methoxy-1-(oxetan-2- ylmethyl)-1H- benzo[d]imidazol-6- yl)isoxazol-3(2H)-one | 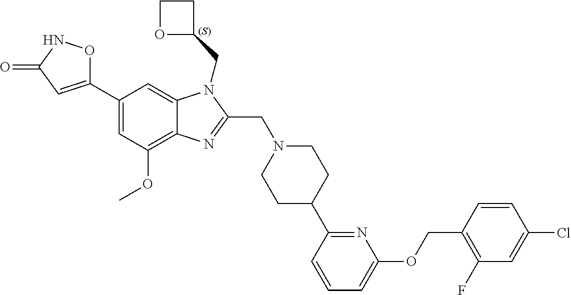 |
| 383 | 2-((4-(6-((4-Chloro-2- fluorobenzyl)oxy)pyridin-2- yl)piperazin-1-yl)methyl)-4- methoxy-1-methyl-1H- benzo[d]imidazole-6- carboxylic acid | 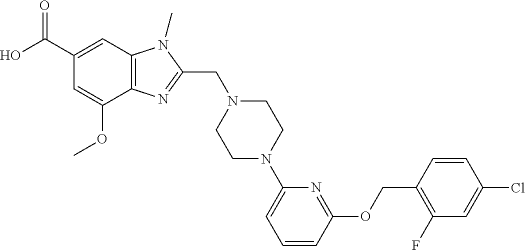 [SMILES: COc1cc(C(=O)O)cc2c1nc(CN1CCN(c3cccc(OCc4ccc(Cl)cc4F)n3)CC1)n2C] |
| 384 | 4-(((6-(1-((4-Ethoxy-1- methyl-6-(3-oxo-2,3- dihydroisoxazol-5-yl)-1H- benzo[d]imidazol-2- yl)methyl)piperidin-4- yl)pyridin-2-yl)oxy)methyl)- 3-fluorobenzonitrile | 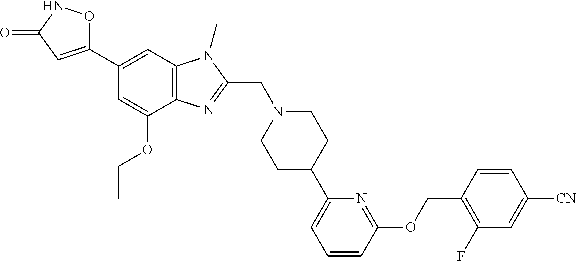 [SMILES: CCOc1cc(-c2cc(=O)[nH]o2)cc2c1nc(CN1CCC(c3cccc(OCc4ccc(C#N)cc4F)n3)CC1)n2C] |
| 385 | 4-(Difluoromethoxy)-1- methyl-2-((4-(6-((4- (trifluoromethyl)benzyl)oxy) pyridin-2-yl)piperidin-1- yl)methyl)-1H- benzo[d]imidazole-6- carboxylic acid | 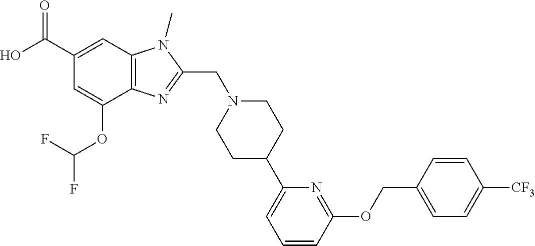 [SMILES: Cn1c(CN2CCC(c3cccc(OCc4ccc(C(F)(F)F)cc4)n3)CC2)nc2c(OC(F)F)cc(C(=O)O)cc21] |
| 386 | 2-((4-((S)-2-(5- Chloropyridin-2-yl)-2- methylbenzo[d][1,3]dioxol- 4-yl)piperidin-1-yl)methyl)- 4-(1,1-difluoroethyl)-1-(((S)- oxetan-2-yl)methyl)-1H- benzo[d]imidazole-6- carboxylic acid | 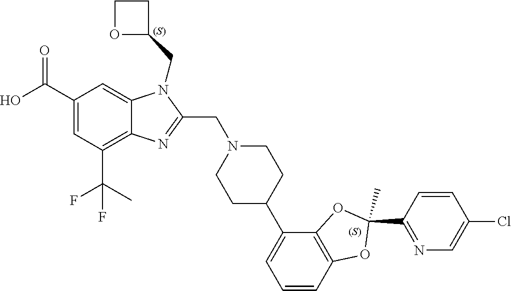 |
| 387 | 2-(((1S,5R,6S)-5-(6-((4- cyano-2- fluorobenzyl)oxy)pyridin-2- yl)-2- azabicyclo[4.1.0]heptan-2- yl)methyl)-4-methoxy-1- (((S)-oxetan-2-yl)methyl)- 1H-benzo[d]imidazole-6- carboxylic acid | 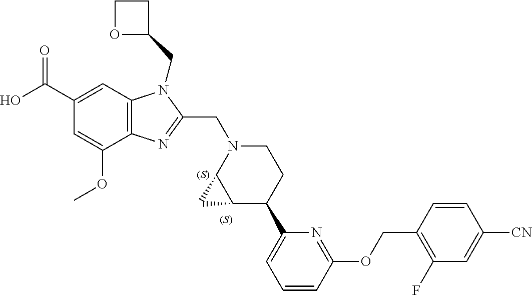 |

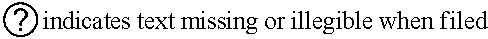

TABLE REF-1

| | |
|---|---|
| REF-1 | 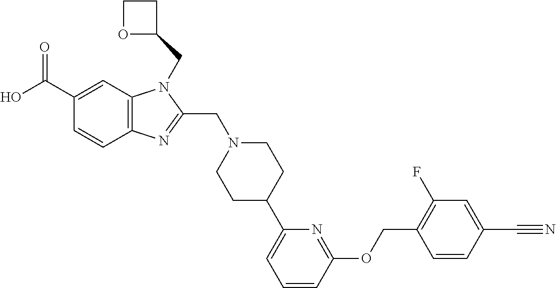 [SMILES: N#Cc1ccc(COc2cccc(C3CCN(Cc4nc5ccc(C(=O)O)cc5n4C[C@@H]4CCO4)CC3)n2)c(F)c1] |

In some embodiments, the compound is a pharmaceutically acceptable salt of any one of the compounds described in Table 1.

In some aspects, the present disclosure provides a compound being an isotopic derivative (e.g., isotopically labeled compound) of any one of the compounds of the Formulae disclosed herein.

In some embodiments, the compound is an isotopic derivative of any one of the compounds described in Table 1 and prodrugs and pharmaceutically acceptable salts thereof.

In some embodiments, the compound is an isotopic derivative of any one of the compounds described in Table 1 and pharmaceutically acceptable salts thereof.

In some embodiments, the compound is an isotopic derivative of any one of prodrugs of the compounds described in Table 1 and pharmaceutically acceptable salts thereof.

In some embodiments, the compound is an isotopic derivative of any one of the compounds described in Table 1.

It is understood that the isotopic derivative can be prepared using any of a variety of art-recognised techniques. For example, the isotopic derivative can generally be prepared by carrying out the procedures disclosed in the Schemes and/or in the Examples described herein, by substituting an isotopically labeled reagent for a non-isotopically labeled reagent.

In some embodiments, the isotopic derivative is a deuterium labeled compound.

In some embodiments, the isotopic derivative is a deuterium labeled compound of any one of the compounds of the Formulae disclosed herein.

The term “isotopic derivative”, as used herein, refers to a derivative of a compound in which one or more atoms are isotopically enriched or labelled. For example, an isotopic derivative of a compound of Formula (I) is isotopically enriched with regard to, or labelled with, one or more isotopes as compared to the corresponding compound of Formula (I). In some embodiments, the isotopic derivative is enriched with regard to, or labelled with, one or more atoms selected from ²H, ¹³C, ¹⁴C, ¹⁵N, ¹⁸O, ²⁹Si, ³¹P, and ³⁴S. In some embodiments, the isotopic derivative is a deuterium labeled compound (i.e., being enriched with ²H with regard to one or more atoms thereof). In some embodiments, the compound is a ¹⁸F labeled compound. In some embodiments, the compound is a ¹²³I labeled compound, a ¹²⁴I labeled compound, a ¹²⁵I labeled compound, a ¹²⁹I labeled compound, a ¹³¹I labeled compound, a ¹³⁵I labeled compound, or any combination thereof. In some embodiments, the compound is a ³³S labeled compound, a ³⁴S labeled compound, a ³⁵S labeled compound, a ³⁶S labeled compound, or any combination thereof.

It is understood that the ¹⁸F, ¹²³I, ¹²⁴I, ¹²⁵I, ¹²⁹I, ¹³¹I, ¹³⁵I, ³²S, ³⁴S, ³⁵S, and/or ³⁶S labeled compound, can be prepared using any of a variety of art-recognised techniques. For example, the deuterium labeled compound can generally be prepared by carrying out the procedures disclosed in the Schemes and/or in the Examples described herein, by substituting a ¹⁸F, ¹²³I, ¹²⁴I, ¹²⁵I, ¹²⁹I, ¹³¹I, ¹³⁵I, ³S, ³⁴S, ³⁵S, and/or ³⁶S labeled reagent for a non-isotope labeled reagent.

A compound of the invention or a pharmaceutically acceptable salt or solvate thereof that contains one or more of the aforementioned ¹⁸F, ¹²³I, ¹²⁴I, ¹²⁵I, ¹²⁹I, ¹³¹I, ¹³⁵I, ³²S, ³⁴S, ³⁵S, and ³⁶S atom(s) is within the scope of the invention. Further, substitution with isotope (e.g., ¹⁸F, ¹²³I, ¹²⁴I, ¹²⁵I, ¹²⁹I, ¹³¹I, ¹³⁵I, ³S, ³⁴S, ³⁵S, and/or ³⁶S) may afford certain therapeutic advantages resulting from greater metabolic stability, e.g., increased in vivo half-life or reduced dosage requirements.

For the avoidance of doubt it is to be understood that, where in this specification a group is qualified by “described herein”, the said group encompasses the first occurring and broadest definition as well as each and all of the particular definitions for that group.

A suitable pharmaceutically acceptable salt of a compound of the disclosure is, for example, an acid-addition salt of a compound of the disclosure which is sufficiently basic, for example, an acid-addition salt with, for example, an inorganic organic acid, for example hydrochloric, hydrobromic, sulfuric, phosphoric, trifluoroacetic, formic, citric methane sulfonate or maleic acid. In addition, a suitable pharmaceutically acceptable salt of a compound of the disclosure which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, diethylamine, trimethylamine, piperidine, morpholine or tris-(2-hydroxyethyl)amine.

It will be understood that the compounds of any one of the Formulae disclosed herein and any pharmaceutically acceptable salts thereof, comprise stereoisomers, mixtures of stereoisomers, polymorphs of all isomeric forms of said compounds.

It will be understood that while compounds disclosed herein may be presented in one particular configuration. Such particular configuration is not to be construed as limiting the disclosure to one or another isomer, tautomer, regioisomer or stereoisomer, nor does it exclude mixtures of isomers, tautomers, regioisomers or stereoisomers. In some embodiments, the presentation of a compound herein in a particular configuration intends to encompass, and to refer to, each of the available isomers, tautomers, regioisomers, and stereoisomers of the compound, or any mixture thereof; while the presentation further intends to refer to the specific configuration of the compound.

It will be understood that while compounds disclosed herein may be presented without specified configuration (e.g., without specified stereochemistry). Such presentation intends to encompass all available isomers, tautomers, regioisomers, and stereoisomers of the compound. In some embodiments, the presentation of a compound herein without specified configuration intends to refer to each of the available isomers, tautomers, regioisomers, and stereoisomers of the compound, or any mixture thereof.

As used herein, the term “isomerism” means compounds that have identical molecular formulae but differ in the sequence of bonding of their atoms or in the arrangement of their atoms in space. Isomers that differ in the arrangement of their atoms in space are termed “stereoisomers.”

Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

As used herein, the term “chiral centre” refers to a carbon atom bonded to four nonidentical substituents.

As used herein, the term “chiral isomer” means a compound with at least one chiral centre. Compounds with more than one chiral centre may exist either as an individual diastereomer or as a mixture of diastereomers, termed “diastereomeric mixture.” When one chiral centre is present, a stereoisomer may be characterised by the absolute configuration (R or S) of that chiral centre. Absolute configuration refers to the arrangement in space of the substituents attached to the chiral centre. The substituents attached to the chiral centre under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

As used herein, the term “geometric isomer” means the diastereomers that owe their existence to hindered rotation about double bonds or a cycloalkyl linker (e.g., 1,3-cyclobutyl). These configurations are differentiated in their names by the prefixes cis and trans, or Z and E, which indicate that the groups are on the same or opposite side of the double bond in the molecule according to the Cahn-Ingold-Prelog rules.

It is to be understood that the compounds of the present disclosure may be depicted as different chiral isomers or geometric isomers. It is also to be understood that when compounds have chiral isomeric or geometric isomeric forms, all isomeric forms are intended to be included in the scope of the present disclosure, and the naming of the compounds does not exclude any isomeric forms, it being understood that not all isomers may have the same level of activity.

It is to be understood that the structures and other compounds discussed in this disclosure include all atropic isomers thereof. It is also to be understood that not all atropic isomers may have the same level of activity.

As used herein, the term “atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

As used herein, the term “tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerisation is possible, a chemical equilibrium of the tautomers will be reached. The exact ratio of the tautomers depends on several factors, including temperature, solvent and pH. The concept of tautomers that are interconvertible by tautomerisations is called tautomerism. Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

It is to be understood that the compounds of the present disclosure may be depicted as different tautomers. It should also be understood that when compounds have tautomeric forms, all tautomeric forms are intended to be included in the scope of the present disclosure, and the naming of the compounds does not exclude any tautomer form. It will be understood that certain tautomers may have a higher level of activity than others.

An enantiomer can be characterised by the absolute configuration of its asymmetric centre and is described by the R- and S-sequencing rules of Cahn and Prelog, or by the manner in which the molecule rotates the plane of polarised light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this disclosure may possess one or more asymmetric centres; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the disclosure may have geometric isomeric centres (E- and Z-isomers). It is to be understood that the present disclosure encompasses all optical, diastereoisomers and geometric isomers and mixtures thereof that possess GLP-1R agonist activity.

The present disclosure also encompasses compounds of the disclosure as defined herein which comprise one or more isotopic substitutions.

It is to be understood that the compounds of any Formula described herein include the compounds themselves, as well as their salts, and their solvates, if applicable. A salt, for example, can be formed between an anion and a positively charged group (e.g., amino) on a substituted compound disclosed herein. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate).

As used herein, the term “pharmaceutically acceptable anion” refers to an anion suitable for forming a pharmaceutically acceptable salt. Likewise, a salt can also be formed between a cation and a negatively charged group (e.g., carboxylate) on a substituted compound disclosed herein. Suitable cations include sodium ion, potassium ion, magnesium ion, calcium ion, and an ammonium cation such as tetramethylammonium ion or diethylamine ion. The substituted compounds disclosed herein also include those salts containing quaternary nitrogen atoms.

It is to be understood that the compounds of the present disclosure, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

As used herein, the term “solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure origin to the reference compound.

As used herein, the term “derivative” refers to compounds that have a common core structure and are substituted with various groups as described herein.

As used herein, the term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include, but are not limited to, acyl sulfonamides, tetrazoles, sulfonates and phosphonates. See, e.g., Patani and LaVoie, Chem. Rev. 96, 3147-3176, 1996.

It is also to be understood that certain compounds of any one of the Formulae disclosed herein may exist in solvated as well as unsolvated forms such as, for example, hydrated forms. A suitable pharmaceutically acceptable solvate is, for example, a hydrate such as hemi-hydrate, a mono-hydrate, a di-hydrate or a tri-hydrate. It is to be understood that the disclosure encompasses all such solvated forms that possess GLP-1R agonist activity.

Compounds of any one of the Formulae disclosed herein may exist in a number of different tautomeric forms and references to compounds of Formula (I) include all such forms. For the avoidance of doubt, where a compound can exist in one of several tautomeric forms, and only one is specifically described or shown, all others are nevertheless embraced by Formula (I). Examples of tautomeric forms include keto-, enol-, and enolate-forms, as in, for example, the following pairs: keto/enol (illustrated below), imine/enamine, amide/imino alcohol, tautomeric amidine/amidine, nitroso/oxime, thioketone/enethiol, and nitro/aci-nitro.

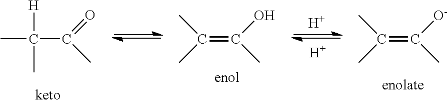

Compounds of any one of the Formulae disclosed herein containing an amine function may also form N-oxides. A reference herein to a compound of Formula (I) that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-oxides can be formed by treatment of the corresponding amine with an oxidising agent such as hydrogen peroxide or a peracid (e.g. a peroxycarboxylic acid), see for example Advanced Organic Chemistry, by Jerry March, 4th Edition, Wiley Interscience, pages. More particularly, N-oxides can be made by the procedure of L. W. Deady (Syn. Comm. 1977, 7, 509-514) in which the amine compound is reacted with meta-chloroperoxybenzoic acid (mCPBA), for example, in an inert solvent such as dichloromethane.

The compounds of any one of the Formulae disclosed herein may be administered in the form of a prodrug which is broken down in the human or animal body to release a compound of the disclosure. A prodrug may be used to alter the physical properties and/or the pharmacokinetic properties of a compound of the disclosure. A prodrug can be formed when the compound of the disclosure contains a suitable group or substituent to which a property-modifying group can be attached. Examples of prodrugs include derivatives containing in vivo cleavable alkyl or acyl substituents at the ester or amide group in any one of the Formulae disclosed herein.

Accordingly, the present disclosure includes those compounds of any one of the Formulae disclosed herein as defined hereinbefore when made available by organic synthesis and when made available within the human or animal body by way of cleavage of a prodrug thereof. Accordingly, the present disclosure includes those compounds of any one of the Formulae disclosed herein that are produced by organic synthetic means and also such compounds that are produced in the human or animal body by way of metabolism of a precursor compound, that is a compound of any one of the Formulae disclosed herein may be a synthetically-produced compound or a metabolically-produced compound.

A suitable pharmaceutically acceptable prodrug of a compound of any one of the Formulae disclosed herein is one that is based on reasonable medical judgment as being suitable for administration to the human or animal body without undesirable pharmacological activities and without undue toxicity. Various forms of prodrug have been described, for example in the following documents: a) Methods in Enzymology, Vol. 42, p. 309-396, edited by K. Widder, et al. (Academic Press, 1985); b) Design of Pro-drugs, edited by H. Bundgaard, (Elsevier, 1985); c) A Textbook of Drug Design and Development, edited by Krogsgaard-Larsen and H. Bundgaard, Chapter 5 “Design and Application of Pro-drugs”, by H. Bundgaard p. 113-191 (1991); d) H. Bundgaard, Advanced Drug Delivery Reviews, 8, 1-38 (1992); e) H. Bundgaard, et al., Journal of Pharmaceutical Sciences, 77, 285 (1988); f) N. Kakeya, et al., Chem. Pharm. Bull., 32, 692 (1984); g) T. Higuchi and V. Stella, “Pro-Drugs as Novel Delivery Systems”, A.C.S. Symposium Series, Volume 14; and h) E. Roche (editor), “Bioreversible Carriers in Drug Design”, Pergamon Press, 1987.

A suitable pharmaceutically acceptable prodrug of a compound of any one of the Formulae disclosed herein that possesses a hydroxy group is, for example, an in vivo cleavable ester or ether thereof. An in vivo cleavable ester or ether of a compound of any one of the Formulae disclosed herein containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C₁-C₁₀ alkanoyl groups such as acetyl, benzoyl, phenylacetyl and substituted benzoyl and phenylacetyl groups, C₁-C₁₀ alkoxycarbonyl groups such as ethoxycarbonyl, N,N—(C₁-C₆ alkyl)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁-C₄ alkyl) piperazin-1-ylmethyl. Suitable pharmaceutically acceptable ether forming groups for a hydroxy group include α-acyloxyalkyl groups such as acetoxymethyl and pivaloyloxymethyl groups.

A suitable pharmaceutically acceptable prodrug of a compound of any one of the Formulae disclosed herein that possesses a carboxy group is, for example, an in vivo cleavable amide thereof, for example an amide formed with an amine such as ammonia, a C₁₋₄alkylamine such as methylamine, a (C₁-C₄ alkyl)₂amine such as dimethylamine, N-ethyl-N-methylamine or diethylamine, a C₁-C₄ alkoxy-C₂-C₄ alkylamine such as 2-methoxyethylamine, a phenyl-C₁-C₄ alkylamine such as benzylamine and amino acids such as glycine or an ester thereof.

A suitable pharmaceutically acceptable prodrug of a compound of any one of the Formulae disclosed herein that possesses an amino group is, for example, an in vivo cleavable amide derivative thereof. Suitable pharmaceutically acceptable amides from an amino group include, for example an amide formed with C₁-C₁₀ alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁-C₄ alkyl) piperazin-1-ylmethyl.

The in vivo effects of a compound of any one of the Formulae disclosed herein may be exerted in part by one or more metabolites that are formed within the human or animal body after administration of a compound of any one of the Formulae disclosed herein. As stated hereinbefore, the in vivo effects of a compound of any one of the Formulae disclosed herein may also be exerted by way of metabolism of a precursor compound (a prodrug).

Suitably, the present disclosure excludes any individual compounds not possessing the biological activity defined herein.

### Pharmaceutical Compositions

In some aspects, the present disclosure provides a pharmaceutical composition comprising a compound of the present disclosure as an active ingredient. In some embodiments, the present disclosure provides a pharmaceutical composition comprising at least one compound of each of the formulae described herein, or a pharmaceutically acceptable salt or solvate thereof, and one or more pharmaceutically acceptable carriers or excipients. In some embodiments, the present disclosure provides a pharmaceutical composition comprising at least one compound selected from Table 1.

As used herein, the term “composition” is intended to encompass a product comprising the specified ingredients in the specified amounts, as well as any product which results, directly or indirectly, from combination of the specified ingredients in the specified amounts.

The compounds of present disclosure can be formulated for oral administration in forms such as tablets, capsules (each of which includes sustained release or timed release formulations), pills, powders, granules, elixirs, tinctures, suspensions, syrups and emulsions. The compounds of present disclosure on can also be formulated for intravenous (bolus or in-fusion), intraperitoneal, topical, subcutaneous, intramuscular or transdermal (e.g., patch) administration, all using forms well known to those of ordinary skill in the pharmaceutical arts.

The formulation of the present disclosure may be in the form of an aqueous solution comprising an aqueous vehicle. The aqueous vehicle component may comprise water and at least one pharmaceutically acceptable excipient. Suitable acceptable excipients include those selected from the group consisting of a solubility enhancing agent, chelating agent, preservative, tonicity agent, viscosity/suspending agent, buffer, and pH modifying agent, and a mixture thereof.

Any suitable solubility enhancing agent can be used. Examples of a solubility enhancing agent include cyclodextrin, such as those selected from the group consisting of hydroxypropyl-β-cyclodextrin, methyl-β-cyclodextrin, randomly methylated-β-cyclodextrin, ethylated-β-cyclodextrin, triacetyl-β-cyclodextrin, peracetylated-β-cyclodextrin, carboxymethyl-β-cyclodextrin, hydroxyethyl-β-cyclodextrin, 2-hydroxy-3-(trimethylammonio) propyl-β-cyclodextrin, glucosyl-β-cyclodextrin, sulfated β-cyclodextrin (S-β-CD), maltosyl-β-cyclodextrin, β-cyclodextrin sulfobutyl ether, branched-β-cyclodextrin, hydroxypropyl-γ-cyclodextrin, randomly methylated-γ-cyclodextrin, and trimethyl-γ-cyclodextrin, and mixtures thereof.

Any suitable chelating agent can be used. Examples of a suitable chelating agent include those selected from the group consisting of ethylenediaminetetraacetic acid and metal salts thereof, disodium edetate, trisodium edetate, and tetrasodium edetate, and mixtures thereof.

Any suitable preservative can be used. Examples of a preservative include those selected from the group consisting of quaternary ammonium salts such as benzalkonium halides (preferably benzalkonium chloride), chlorhexidine gluconate, benzethonium chloride, cetyl pyridinium chloride, benzyl bromide, phenylmercury nitrate, phenylmercury acetate, phenylmercury neodecanoate, merthiolate, methylparaben, propylparaben, sorbic acid, potassium sorbate, sodium benzoate, sodium propionate, ethyl p-hydroxybenzoate, propylaminopropyl biguanide, and butyl-p-hydroxybenzoate, and sorbic acid, and mixtures thereof.

The aqueous vehicle may also include a tonicity agent to adjust the tonicity (osmotic pressure). The tonicity agent can be selected from the group consisting of a glycol (such as propylene glycol, diethylene glycol, triethylene glycol), glycerol, dextrose, glycerin, mannitol, potassium chloride, and sodium chloride, and a mixture thereof.

The aqueous vehicle may also contain a viscosity/suspending agent. Suitable viscosity/suspending agents include those selected from the group consisting of cellulose derivatives, such as methyl cellulose, ethyl cellulose, hydroxyethylcellulose, polyethylene glycols (such as polyethylene glycol 300, polyethylene glycol 400), carboxymethyl cellulose, hydroxypropylmethyl cellulose, and cross-linked acrylic acid polymers (carbomers), such as polymers of acrylic acid cross-linked with polyalkenyl ethers or divinyl glycol (Carbopols-such as Carbopol 934, Carbopol 934P, Carbopol 971, Carbopol 974 and Carbopol 974P), and a mixture thereof.

In order to adjust the formulation to an acceptable pH (typically a pH range of about 5.0 to about 9.0, more preferably about 5.5 to about 8.5, particularly about 6.0 to about 8.5, about 7.0 to about 8.5, about 7.2 to about 7.7, about 7.1 to about 7.9, or about 7.5 to about 8.0), the formulation may contain a pH modifying agent. The pH modifying agent is typically a mineral acid or metal hydroxide base, selected from the group of potassium hydroxide, sodium hydroxide, and hydrochloric acid, and mixtures thereof, and preferably sodium hydroxide and/or hydrochloric acid. These acidic and/or basic pH modifying agents are added to adjust the formulation to the target acceptable pH range. Hence it may not be necessary to use both acid and base-depending on the formulation, the addition of one of the acid or base may be sufficient to bring the mixture to the desired pH range.

The aqueous vehicle may also contain a buffering agent to stabilise the pH. When used, the buffer is selected from the group consisting of a phosphate buffer (such as sodium dihydrogen phosphate and disodium hydrogen phosphate), a borate buffer (such as boric acid, or salts thereof including disodium tetraborate), a citrate buffer (such as citric acid, or salts thereof including sodium citrate), and ¿-aminocaproic acid, and mixtures thereof.

The formulation may further comprise a wetting agent. Suitable classes of wetting agents include those selected from the group consisting of polyoxypropylene-polyoxyethylene block copolymers (poloxamers), polyethoxylated ethers of castor oils, polyoxyethylenated sorbitan esters (polysorbates), polymers of oxyethylated octyl phenol (Tyloxapol), polyoxyl 40 stearate, fatty acid glycol esters, fatty acid glyceryl esters, sucrose fatty esters, and polyoxyethylene fatty esters, and mixtures thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavouring agent such as peppermint, methyl salicylate, orange flavoring.

According to a further aspect of the disclosure there is provided a pharmaceutical composition which comprises a compound of the disclosure as defined hereinbefore, or a pharmaceutically acceptable salt, hydrate or solvate thereof, in association with a pharmaceutically acceptable diluent or carrier.

The compositions of the disclosure may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the disclosure may be obtained by conventional procedures using conventional pharmaceutical excipients, well known in the art. Thus, compositions intended for oral use may contain, for example, one or more colouring, sweetening, flavouring and/or preservative agents.

An effective amount of a compound of the present disclosure for use in therapy is an amount sufficient to treat or prevent a GLP-1R related condition referred to herein, slow its progression and/or reduce the symptoms associated with the condition.

An effective amount of a compound of the present disclosure for use in therapy is an amount sufficient to treat a GLP-1R related condition referred to herein, slow its progression and/or reduce the symptoms associated with the condition.

The size of the dose for therapeutic or prophylactic purposes of a compound of Formula (I) will naturally vary according to the nature and severity of the conditions, the age and sex of the animal or patient and the route of administration, according to well-known principles of medicine.

### Methods of Use

In some aspects, the present disclosure provides a method of modulating GLP-1R activity (e.g., in vitro or in vivo), comprising contacting a cell with an effective amount of a compound of the present disclosure or a pharmaceutically acceptable salt thereof.

In some aspects, the present disclosure provides a method of modulating GLP-1R activity (e.g., in vitro or in vivo), comprising contacting a cell with a compound of the present disclosure or a pharmaceutically acceptable salt thereof.

In some aspects, the present disclosure provides a method of modulating GLP-1R activity (e.g., in vitro or in vivo), comprising contacting a cell with a compound of the present disclosure or a pharmaceutically acceptable salt thereof.

In some aspects, the present disclosure provides a method of treating or preventing a disease or disorder disclosed herein in a subject in need thereof, comprising administering to the subject a therapeutically effective amount of a compound of the present disclosure or a pharmaceutically acceptable salt thereof.

In some aspects, the present disclosure provides a method of treating a disease or disorder disclosed herein in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing a disease or disorder disclosed herein in a subject in need thereof, comprising administering to the subject a compound of the present disclosure or a pharmaceutically acceptable salt thereof.

In some aspects, the present disclosure provides a method of treating a disease or disorder disclosed herein in a subject in need thereof, comprising administering to the subject a compound of the present disclosure or a pharmaceutically acceptable salt thereof, or a pharmaceutical composition of the present disclosure.

In some embodiments, the disease or disorder is associated with an implicated GLP-1R activity. In some embodiments, the disease or disorder is a disease or disorder in which GLP-1R activity is implicated.

In some embodiments, the disease or disorder is diabetes, NASH, insulinoma, obesity, and/or hyperglycemia.

In some aspects, the present disclosure provides a method of treating or preventing diabetes, NASH, insulinoma, obesity, and/or hyperglycemia in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating diabetes, NASH, insulinoma, obesity, and/or hyperglycemia in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing diabetes, NASH, insulinoma, obesity, and/or hyperglycemia in a subject in need thereof, comprising administering to the subject a compound of the present disclosure or a pharmaceutically acceptable salt thereof, or a pharmaceutical composition of the present disclosure.

In some aspects, the present disclosure provides a method of treating diabetes, NASH, insulinoma, obesity, and/or hyperglycemia in a subject in need thereof, comprising administering to the subject a compound of the present disclosure or a pharmaceutically acceptable salt thereof, or a pharmaceutical composition of the present disclosure.

In some aspects, the present disclosure provides a compound of the present disclosure or a pharmaceutically acceptable salt thereof for use in modulating GLP-1R activity (e.g., in vitro or in vivo).

In some aspects, the present disclosure provides a compound of the present disclosure or a pharmaceutically acceptable salt thereof for use in modulating GLP-1R activity (e.g., in vitro or in vivo).

In some aspects, the present disclosure provides a compound of the present disclosure or a pharmaceutically acceptable salt thereof for use in treating or preventing a disease or disorder disclosed herein.

In some aspects, the present disclosure provides a compound of the present disclosure or a pharmaceutically acceptable salt thereof for use in treating a disease or disorder disclosed herein.

In some aspects, the present disclosure provides a compound of the present disclosure or a pharmaceutically acceptable salt thereof for use in treating or preventing diabetes, NASH, insulinoma, obesity, and/or hyperglycemia in a subject in need thereof.

In some aspects, the present disclosure provides a compound of the present disclosure or a pharmaceutically acceptable salt thereof for use in treating diabetes, NASH, insulinoma, obesity, and/or hyperglycemia in a subject in need thereof.

In some aspects, the present disclosure provides use of a compound of the present disclosure or a pharmaceutically acceptable salt thereof in the manufacture of a medicament for modulating GLP-1R activity (e.g., in vitro or in vivo).

In some aspects, the present disclosure provides use of a compound of the present disclosure or a pharmaceutically acceptable salt thereof in the manufacture of a medicament for treating or preventing a disease or disorder disclosed herein.

In some aspects, the present disclosure provides use of a compound of the present disclosure or a pharmaceutically acceptable salt thereof in the manufacture of a medicament for treating a disease or disorder disclosed herein.

In some aspects, the present disclosure provides use of a compound of the present disclosure or a pharmaceutically acceptable salt thereof in the manufacture of a medicament for treating or preventing diabetes, NASH, insulinoma, obesity, and/or hyperglycemia in a subject in need thereof.

In some aspects, the present disclosure provides use of a compound of the present disclosure or a pharmaceutically acceptable salt thereof in the manufacture of a medicament for treating cancer in a subject in need thereof.

The present disclosure provides compounds that function as modulators of GLP-1R activity.

In some embodiments, the compounds of the present disclosure are agonists of the GLP-1 receptor.

In some embodiments, the modulation of the GLP-1R receptor is activation of the GLP-1R receptor.

Effectiveness of compounds of the disclosure can be determined by industry-accepted assays/disease models according to standard practices of elucidating the same as described in the art and are found in the current general knowledge.

The present disclosure also provides a method of treating a disease or disorder in which GLP-1R activity is implicated in a patient in need of such treatment, said method comprising administering to said patient a therapeutically effective amount of a compound, or a pharmaceutically acceptable salt thereof, or a pharmaceutical composition as defined herein.

In accordance with the present application, a disease or condition to be treated and/or prevented is selected from the group consisting of cardiometabolic and associated diseases including diabetes (T1 D and/or T2DM, including pre-diabetes), idiopathic T1 D (Type 1 b), latent autoimmune diabetes in adults (LADA), early-onset T2DM (EOD), youth-onset atypical diabetes (YOAD), maturity onset diabetes of the young (MODY), malnutrition-related diabetes, gestational diabetes, hyperglycemia, insulin resistance, hepatic insulin resistance, impaired glucose tolerance, diabetic neuropathy, diabetic nephropathy, kidney disease (e.g., acute kidney disorder, tubular dysfunction, proinflammatory changes to the proximal tubules), diabetic retinopathy, adipocyte dysfunction, visceral adipose deposition, sleep apnea, obesity (including hypothalamic obesity and monogenic obesity) and related comorbidities (e.g., osteoarthritis and urine incontinence), eating disorders (including binge eating syndrome, bulimia nervosa, and syndromic obesity such as Prader-Willi and Bardet-Biedl syndromes), weight gain from use of other agents (e.g., from use of steroids and antipsychotics), excessive sugar craving, dyslipidemia (including hyperlipidemia, hypertriglyceridemia, increased total cholesterol, high LDL cholesterol, and low HDL cholesterol), hyperinsulinemia, liver diseases such as NAFLD, steatosis, NASH, fibrosis, cirrhosis, and hepatocellular carcinoma, cardiovascular disease, atherosclerosis (including coronary artery disease), peripheral vascular disease, hypertension, endothelial dysfunction, impaired vascular compliance, congestive heart failure, myocardial infarction (e.g. necrosis and apoptosis), stroke, hemorrhagic stroke, ischemic stroke, traumatic brain injury, pulmonary hypertension, restenosis after angioplasty, intermittent claudication, post-prandial lipemia, metabolic acidosis, ketosis, arthritis, osteoporosis, Parkinson's Disease, left ventricular hypertrophy, peripheral arterial disease, macular degeneration, cataract, glomerulosclerosis, chronic renal failure, metabolic syndrome, syndrome X, premenstrual syndrome, angina pectoris, thrombosis, atherosclerosis, transient ischemic attacks, vascular restenosis, impaired glucose metabolism, conditions of impaired fasting plasma glucose, hyperuricemia, gout, erectile dysfunction, skin and connective tissue disorders, psoriasis, foot ulcerations, ulcerative colitis, hyper apo B lipoproteinemia, Alzheimer's Disease, schizophrenia, impaired cognition, inflammatory bowel disease, short bowel syndrome, Crohn's disease, colitis, irritable bowel syndrome, Polycystic Ovary Syndrome and addiction (e.g., alcohol and/or drug abuse), prevention or treatment of Polycystic Ovary Syndrome and treatment of addiction (e.g., alcohol and/or drug abuse).

In some embodiments, provided herein is a method of treating a cardiometabolic disease in a subject (e.g., a human patient) in need thereof, comprising administering to the subject a therapeutically effective amount of a compound described herein, or a pharmaceutically acceptable salt thereof.

In some embodiments, provided herein is a method of treating diabetes in a subject (e.g., a human patient) in need thereof, comprising administering to the subject a therapeutically effective amount of a compound described herein, or a pharmaceutically acceptable salt thereof. Exemplary diabetes include, but are not limited to, T1 D, T2DM, pre-diabetes, idiopathic T1 D, LADA, EOD, YOAD, MODY, malnutrition-related diabetes, and gestational diabetes.

In some embodiments, provided herein is a method of treating a liver disorder in a subject (e.g., a human patient) in need thereof, comprising administering to the subject a therapeutically effective amount of a compound described herein, or a pharmaceutically acceptable salt thereof. Exemplary liver disorders include, without limitation, liver inflammation, fibrosis, and steatohepatitis. In some embodiments, the liver disorder is selected from the list consisting of primary biliary cirrhosis (PBC), primary sclerosing cholangitis (PSC), drug induced cholestasis, intrahepatic cholestasis of pregnancy, parenteral nutrition associated cholestasis (PNAC), bacterial overgrowth or sepsis associated cholestasis, autoimmune hepatitis, viral hepatitis, alcoholic liver disease, nonalcoholic fatty liver disease (NAFLD), nonalcoholic steatohepatitis (NASH), graft versus host disease, transplant liver regeneration, congenital hepatic fibrosis, choledocholithiasis, granulomatous liver disease, intra- or extrahepatic malignancy, Sjogren's syndrome, sarcoidosis, Wilson's disease, Gaucher's disease, hemochromatosis, and oti-antitrypsin deficiency. In some embodiments, the liver disorder is selected from the list consisting of liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH). In some embodiments, the liver disorder is selected from the group consisting of liver fibrosis, alcohol induced fibrosis, steatosis, alcoholic steatosis, NAFLD, and NASH. In one embodiment, the liver disorder is NASH. In another embodiment, the liver disorder is liver inflammation. In another embodiment, the liver disorder is liver fibrosis. In another embodiment, the liver disorder is alcohol induced fibrosis. In another embodiment, the liver disorder is steatosis. In another embodiment, the liver disorder is alcoholic steatosis. In another embodiment, the liver disorder is NAFLD. In one embodiment, the treatment methods provided herein impedes or slows the progression of NAFLD to NASH. In one embodiment, the treatment methods provided herein impedes or slows the progression of NASH. NASH can progress, e.g., to one or more of liver cirrhosis, hepatic cancer, etc. In some embodiments, the liver disorder is NASH. In some embodiments, the patient has had a liver biopsy. In some embodiments, the method further comprising obtaining the results of a liver biopsy.

In accordance with the present application, a compound described herein, or a pharmaceutically acceptable salt thereof, can be administered by any suitable route in the form of a pharmaceutical composition adapted to such a route, and in a dose effective for the treatment intended. In some embodiments, it is a compound of any embodiment of Formula (I) or a sub-formula thereof, or selected from the compounds of Table 1, or a pharmaceutically acceptable salt thereof. The compounds and/or compositions described herein may be administered orally, rectally, vaginally, parenterally, or topically.

In some embodiments, the compounds and/or compositions may be administered orally. Oral administration may involve swallowing, so that the compound enters the gastrointestinal tract, or buccal or sublingual administration may be employed by which the compound enters the bloodstream directly from the mouth.

In some embodiments, the compounds and/or compositions may be administered directly into the bloodstream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

In some embodiments, the compounds and/or compositions may be administered topically to the skin or mucosa, that is, dermally or transdermally. In some embodiments, the compounds and/or compositions may be administered intranasally or by inhalation. In some embodiments, the compounds and/or compositions may be administered rectally or vaginally. In some embodiments, the compounds and/or compositions may be administered directly to the eye or ear.

The compounds and/or compositions described herein can be used alone, or in combination with other therapeutic agents. The administration of two or more agents “in combination” means that all of the agents are administered closely enough in time that each may generate a biological effect in the same time frame. The presence of one agent may alter the biological effects of the other agent(s). The two or more agents may be administered simultaneously, concurrently or sequentially. Additionally, simultaneous administration may be carried out by mixing the agents prior to administration or by administering the compounds at the same point in time but as separate dosage forms at the same or different site of administration.

In some embodiments, the one or more other therapeutic agent is an anti-diabetic agent including but not limited to a biguanide (e.g., metformin), a sulfonylurea (e.g., tolbutamide, glibenclamide, gliclazide, chlorpropamide, tolazamide, acetohexamide·glyclopyramide, glimepiride, or glipizide), a thiazolidinedione (e.g., pioglitazone, rosiglitazone, or lobeglitazone), a glitazar (e.g., saroglitazar, aleglitazar, muraglitazar or tesaglitazar), a meglitinide (e.g., nateglinide, repaglinide), a dipeptidyl peptidase 4 (DPP-4) inhibitor (e.g., sitagliptin, vildagliptin, saxagliptin, linagliptin, gemigliptin, anagliptin, teneligliptin, alogliptin, trelagliptin, dutogliptin, or omarigliptin), a glitazone (e.g., pioglitazone, rosiglitazone, balaglitazone, rivoglitazone, or lobeglitazone), a sodium-glucose linked transporter 2 (SGLT2) inhibitor (e.g., empagliflozin, canagliflozin, dapagliflozin, ipragliflozin, Ipragliflozin, tofogliflozin, sergliflozin etabonate, remogliflozin etabonate, or ertugliflozin), an SGLTL1 inhibitor, a GPR40 agonist (FFAR1/FFA1 agonist, e.g. fasiglifam), glucose-dependent insulinotropic peptide (GIP) and analogues thereof, an alpha glucosidase inhibitor (e.g. voglibose, acarbose, or miglitol), or an insulin or an insulin analogue, including the pharmaceutically acceptable salts of the specifically named agents and the pharmaceutically acceptable solvates of said agents and salts.

In some embodiments, the one or more other therapeutic agent is an antiobesity agent including but not limited to peptide YY or an analogue thereof, a neuropeptide Y receptor type 2 (NPYR2) agonist, a NPYR1 or NPYR5 antagonist, a cannabinoid receptor type 1 (CB1 R) antagonist, a lipase inhibitor (e.g., orlistat), a human proislet peptide (HIP), a melanocortin receptor 4 agonist (e.g., setmelanotide), a melanin concentrating hormone receptor 1 antagonist, a farnesoid X receptor (FXR) agonist (e.g. obeticholic acid), zonisamide, phentermine (alone or in combination with topiramate), a norepinephrine/dopamine reuptake inhibitor (e.g., buproprion), an opioid receptor antagonist (e.g., naltrexone), a combination of norepinephrine/dopamine reuptake inhibitor and opioid receptor antagonist (e.g., a combination of bupropion and naltrexone), a GDF-15 analog, sibutramine, a cholecystokinin agonist, amylin and analogues thereof (e.g., pramlintide), leptin and analogues thereof (e.g., metroleptin), a serotonergic agent (e.g., lorcaserin), a methionine aminopeptidase 2 (MetAP2) inhibitor (e.g., beloranib or ZGN-1061), phendimetrazine, diethylpropion, benzphetamine, an SGLT2 inhibitor (e.g., empagliflozin, canagliflozin, dapagliflozin, ipragliflozin, Ipragliflozin, tofogliflozin, sergliflozin etabonate, remogliflozin etabonate, or ertugliflozin), an SGLTL1 inhibitor, a dual SGLT2/SGLT1 inhibitor, a fibroblast growth factor receptor (FGFR) modulator, an AMP-activated protein kinase (AMPK) activator, biotin, a MAS receptor modulator, or a glucagon receptor agonist (alone or in combination with another GLP-1 R agonist, e.g., liraglutide, exenatide, dulaglutide, albiglutide, lixisenatide, or semaglutide), including the pharmaceutically acceptable salts of the specifically named agents and the pharmaceutically acceptable solvates of said agents and salts.

In some embodiments, the one or more other therapeutic agent is an agent to treat NASH including but not limited to PF-05221304, an FXR agonist (e.g., obeticholic acid), a PPAR a/d agonist (e.g., elafibranor), a synthetic fatty acid-bile acid conjugate (e.g., aramchol), a caspase inhibitor (e.g., emricasan), an anti-lysyl oxidase homologue 2 (LOXL2) monoclonal antibody (e.g., simtuzumab), a galectin 3 inhibitor (e.g., GR-MD-02), a MAPK5 inhibitor (e.g., GS-4997), a dual antagonist of chemokine receptor 2 (CCR2) and CCR5 (e.g., cenicriviroc), a fibroblast growth factor21 (FGF21) agonist (e.g., BMS-986036), a leukotriene D4 (LTD4) receptor antagonist (e.g., tipelukast), a niacin analogue (e.g., ARI 3037MO), an ASBT inhibitor (e.g., volixibat), an acetyl-CoA carboxylase (ACC) inhibitor (e.g., NDI 010976), a ketohexokinase (KHK) inhibitor, a diacylglyceryl acyltransferase 2 (DGAT2) inhibitor, a CB1 receptor antagonist, an anti-CB1 R antibody, or an apoptosis signal-regulating kinase 1 (ASK1) inhibitor, including the pharmaceutically acceptable salts of the specifically named agents and the pharmaceutically acceptable solvates of said agents and salts.

### Articles of Manufacture and Kits

The present disclosure further provides articles of manufacture comprising a compound, or a pharmaceutically acceptable salt thereof in accordance with the present application, a composition described herein, or one or more unit dosages described herein in suitable packaging. In certain embodiments, the article of manufacture is for use in any of the methods described herein. Suitable packaging (e.g., containers) is known in the art and includes, for example, vials, vessels, ampules, bottles, jars, flexible packaging and the like. An article of manufacture may further be sterilized and/or sealed.

The kits may be in unit dosage forms, bulk packages (e.g., multi-dose packages) or sub-unit doses. For example, kits may be provided that contain sufficient dosages of a compound, or a pharmaceutically acceptable salt thereof in accordance with the present application, a composition described herein, and/or one or more other therapeutic agent useful for a disease detailed herein to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the compounds/compositions described herein and instructions for use and be packaged in quantities sufficient for storage and use in pharmacies (e.g., hospital pharmacies and compounding pharmacies

The kits may optionally include a set of instructions, generally written instructions, although electronic storage media (e.g., magnetic diskette or optical disk) containing instructions are also acceptable, relating to the use of component(s) of the methods of the present disclosure. The instructions included with the kit generally include information as to the components and their administration to an individual.

### Routes of Administration

Compounds of the present disclosure, or pharmaceutically acceptable salts thereof, may be administered alone as a sole therapy or can be administered in addition with one or more other substances and/or treatments. Such conjoint treatment may be achieved by way of the simultaneous, sequential or separate administration of the individual components of the treatment.

For example, therapeutic effectiveness may be enhanced by administration of an adjuvant (i.e. by itself the adjuvant may only have minimal therapeutic benefit, but in combination with another therapeutic agent, the overall therapeutic benefit to the individual is enhanced). Alternatively, by way of example only, the benefit experienced by an individual may be increased by administering the compound of Formula (I) with another therapeutic agent (which also includes a therapeutic regimen) that also has therapeutic benefit.

In the instances where the compound of the present disclosure is administered in combination with other therapeutic agents, the compound of the disclosure need not be administered via the same route as other therapeutic agents, and may, because of different physical and chemical characteristics, be administered by a different route. For example, the compound of the disclosure may be administered orally to generate and maintain good blood levels thereof, while the other therapeutic agent may be administered intravenously. The initial administration may be made according to established protocols known in the art, and then, based upon the observed effects, the dosage, modes of administration and times of administration can be modified by the skilled clinician.

The particular choice of other therapeutic agent will depend upon the diagnosis of the attending physicians and their judgment of the condition of the individual and the appropriate treatment protocol. According to this aspect of the disclosure there is provided a combination for use in the treatment of a disease in which GLP-1R activity is implicated comprising a compound of the disclosure as defined hereinbefore, or a pharmaceutically acceptable salt thereof, and another suitable agent.

According to a further aspect of the disclosure there is provided a pharmaceutical composition which comprises a compound of the disclosure, or a pharmaceutically acceptable salt thereof, in combination with a suitable, in association with a pharmaceutically acceptable diluent or carrier.

In addition to its use in therapeutic medicine, compounds of Formula (I) and pharmaceutically acceptable salts thereof are also useful as pharmacological tools in the development and standardisation of in vitro and in vivo test systems for the evaluation of the effects of modulators of GLP-1R activity in laboratory animals such as dogs, rabbits, monkeys, mini-pigs, rats and mice, as part of the search for new therapeutic agents.

In any of the above-mentioned pharmaceutical composition, process, method, use, medicament, and manufacturing features of the instant disclosure, any of the alternate embodiments of macromolecules of the present disclosure described herein also apply.

The compounds of the disclosure or pharmaceutical compositions comprising these compounds may be administered to a subject by any route of administration, whether systemically/peripherally or topically (i.e., at the site of desired action).

Routes of administration include, but are not limited to, oral (e.g. by ingestion); buccal; sublingual; transdermal (including, e.g., by a patch, plaster, etc.); transmucosal (including, e.g., by a patch, plaster, etc.); intranasal (e.g., by nasal spray or powder); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### Methods of Synthesis

In some aspects, the present disclosure provides a method of preparing a compound of the present disclosure.

In some aspects, the present disclosure provides a method of a compound, comprising one or more steps as described herein.

In some aspects, the present disclosure provides a compound obtainable by, or obtained by, or directly obtained by a method for preparing a compound as described herein.

In some aspects, the present disclosure provides an intermediate as described herein, being suitable for use in a method for preparing a compound as described herein.

The compounds of the present disclosure can be prepared by any suitable technique known in the art. Particular processes for the preparation of these compounds are described further in the accompanying examples.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

It will be appreciated that during the synthesis of the compounds of the disclosure in the processes defined herein, or during the synthesis of certain starting materials, it may be desirable to protect certain substituent groups to prevent their undesired reaction. The skilled chemist will appreciate when such protection is required, and how such protecting groups may be put in place, and later removed. For examples of protecting groups see one of the many general texts on the subject, for example, ‘Protective Groups in Organic Synthesis’ by Theodora Green (publisher: John Wiley & Sons). Protecting groups may be removed by any method described in the literature or known to the skilled chemist as appropriate for the removal of the protecting group in question, such methods being chosen so as to effect removal of the protecting group with the minimum disturbance of groups elsewhere in the molecule. Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl, or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium on carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively an arylmethyl group such as a benzyl group may be removed, for example, by hydrogenation over a catalyst such as palladium on carbon.

A suitable protecting group for a carboxy group is, for example, an esterifying group, for example a methyl or an ethyl group which may be removed, for example, by hydrolysis with a base such as sodium hydroxide, or for example a tert-butyl group which may be removed, for example, by treatment with an acid, for example an organic acid such as trifluoroacetic acid, or for example a benzyl group which may be removed, for example, by hydrogenation over a catalyst such as palladium on carbon.

Once a compound of Formula (I) has been synthesised by any one of the processes defined herein, the processes may then further comprise the additional steps of: (i) removing any protecting groups present; (ii) converting the compound Formula (I) into another compound of Formula (I); (iii) forming a pharmaceutically acceptable salt, hydrate or solvate thereof; and/or (iv) forming a prodrug thereof.

The resultant compounds of Formula (I) can be isolated and purified using techniques well known in the art.

In some embodiments, the reaction of the compounds is carried out in the presence of a suitable solvent, which is preferably inert under the respective reaction conditions. Examples of suitable solvents comprise but are not limited to hydrocarbons, such as hexane, petroleum ether, benzene, toluene or xylene; chlorinated hydrocarbons, such as trichlorethylene, 1,2-dichloroethane, tetrachloromethane, chloroform or dichloromethane; alcohols, such as methanol, ethanol, isopropanol, n-propanol, n-butanol or tert-butanol; ethers, such as diethyl ether, diisopropyl ether, tetrahydrofuran (THF), 2-methyltetrahydrofuran, cyclopentylmethyl ether (CPME), methyl tert-butyl ether (MTBE) or dioxane; glycol ethers, such as ethylene glycol monomethyl or monoethyl ether or ethylene glycol dimethyl ether (diglyme); ketones, such as acetone, methylisobutylketone (MIBK) or butanone; amides, such as acetamide, dimethylacetamide, dimethylformamide (DMF) or N-methylpyrrolidinone (NMP); nitriles, such as acetonitrile; sulfoxides, such as dimethyl sulfoxide (DMSO); nitro compounds, such as nitromethane or nitrobenzene; esters, such as ethyl acetate or methyl acetate, or mixtures of the said solvents or mixtures with water.

The reaction temperature is suitably between about −100° C. and 300° C., depending on the reaction step and the conditions used.

Reaction times are generally in the range between a fraction of a minute and several days, depending on the reactivity of the respective compounds and the respective reaction conditions. Suitable reaction times are readily determinable by methods known in the art, for example reaction monitoring. Based on the reaction temperatures given above, suitable reaction times generally lie in the range between 10 minutes and 48 hours.

Moreover, by utilising the procedures described herein, in conjunction with ordinary skills in the art, additional compounds of the present disclosure can be readily prepared. Those skilled in the art will readily understand that known variations of the conditions and processes of the following preparative procedures can be used to prepare these compounds.

As will be understood by the person skilled in the art of organic synthesis, compounds of the present disclosure are readily accessible by various synthetic routes, some of which are exemplified in the accompanying examples. The skilled person will easily recognise which kind of reagents and reactions conditions are to be used and how they are to be applied and adapted in any particular instance—wherever necessary or useful—in order to obtain the compounds of the present disclosure. Furthermore, some of the compounds of the present disclosure can readily be synthesised by reacting other compounds of the present disclosure under suitable conditions, for instance, by converting one particular functional group being present in a compound of the present disclosure, or a suitable precursor molecule thereof, into another one by applying standard synthetic methods, like reduction, oxidation, addition or substitution reactions; those methods are well known to the skilled person. Likewise, the skilled person will apply—whenever necessary or useful—synthetic protecting (or protective) groups; suitable protecting groups as well as methods for introducing and removing them are well-known to the person skilled in the art of chemical synthesis and are described, in more detail, in, e.g., P. G. M. Wuts, T. W. Greene, “Greene's Protective Groups in Organic Synthesis”, 4th edition (2006) (John Wiley & Sons).

General routes for the preparation of a compound of the application are described in Schemes A to W herein.

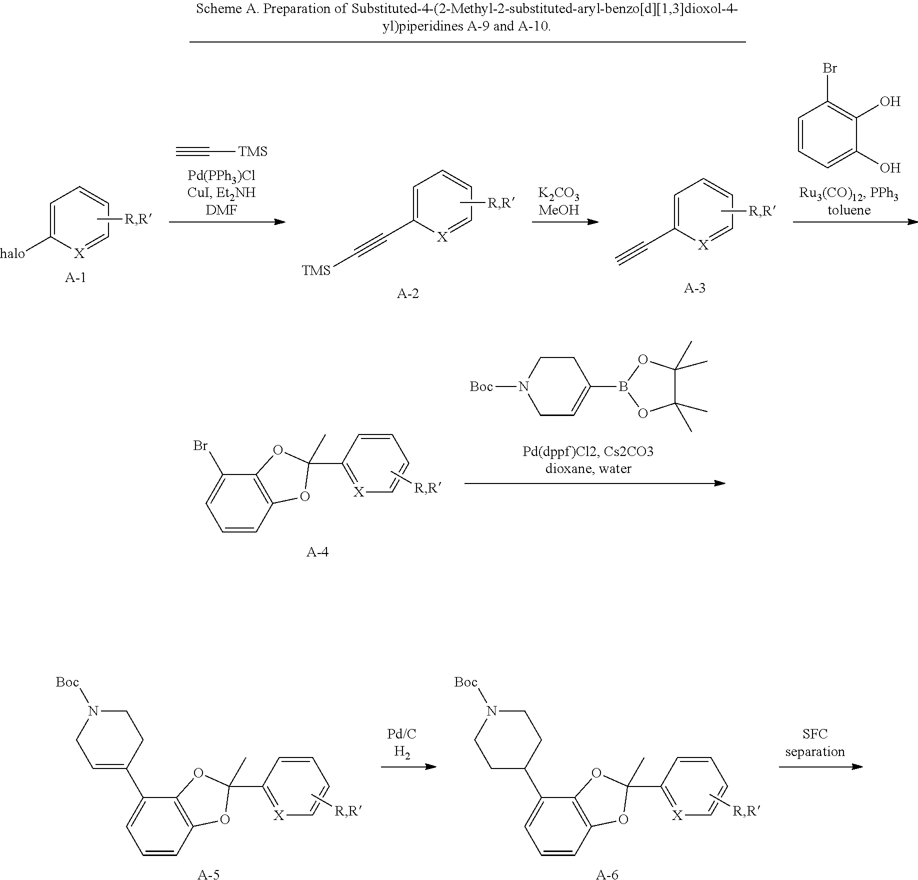

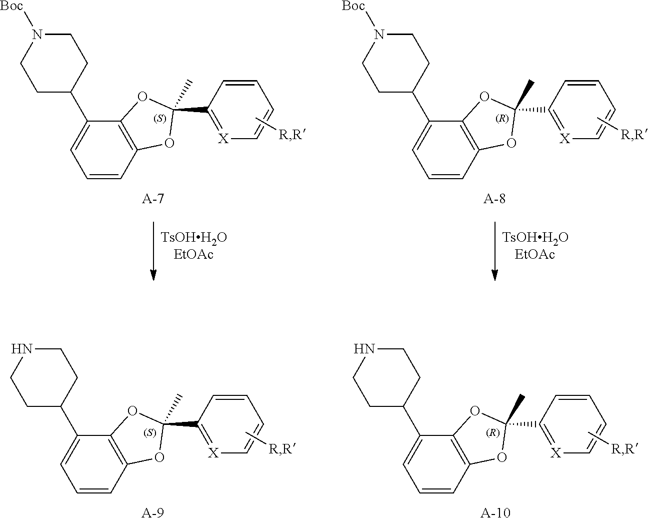

Substituted trimethyl(arylethynyl)silanes A-3 may be prepared via Sonogashira coupling of ethynyltrimethylsilane with substituted aryl halides A-1 followed by silyl deprotection. The addition of 3-bromocatechol to alkyne A-3 in the presence of triruthenium dodecacarbonyl provides 4-bromo-2-methyl-2-arylbenzo[d][1,3]dioxole A-4 as a mixture of enantiomers. Suzuki coupling of A-4 with commercially available tert-butyl 4-(4, 4, 5, 5-tetramethyl-1,3,2-dioxaborolan-2-yl)-3,6-dihydropyridine-1(2H)-carboxylate followed by reduction of the dihydropyridine intermediate provides A-6 which may be separated by super critical fluid chromatography (SFC). Enantiomers A-7 and A-8 are deprotected with strong acid to provide amines A-9 and A-10.

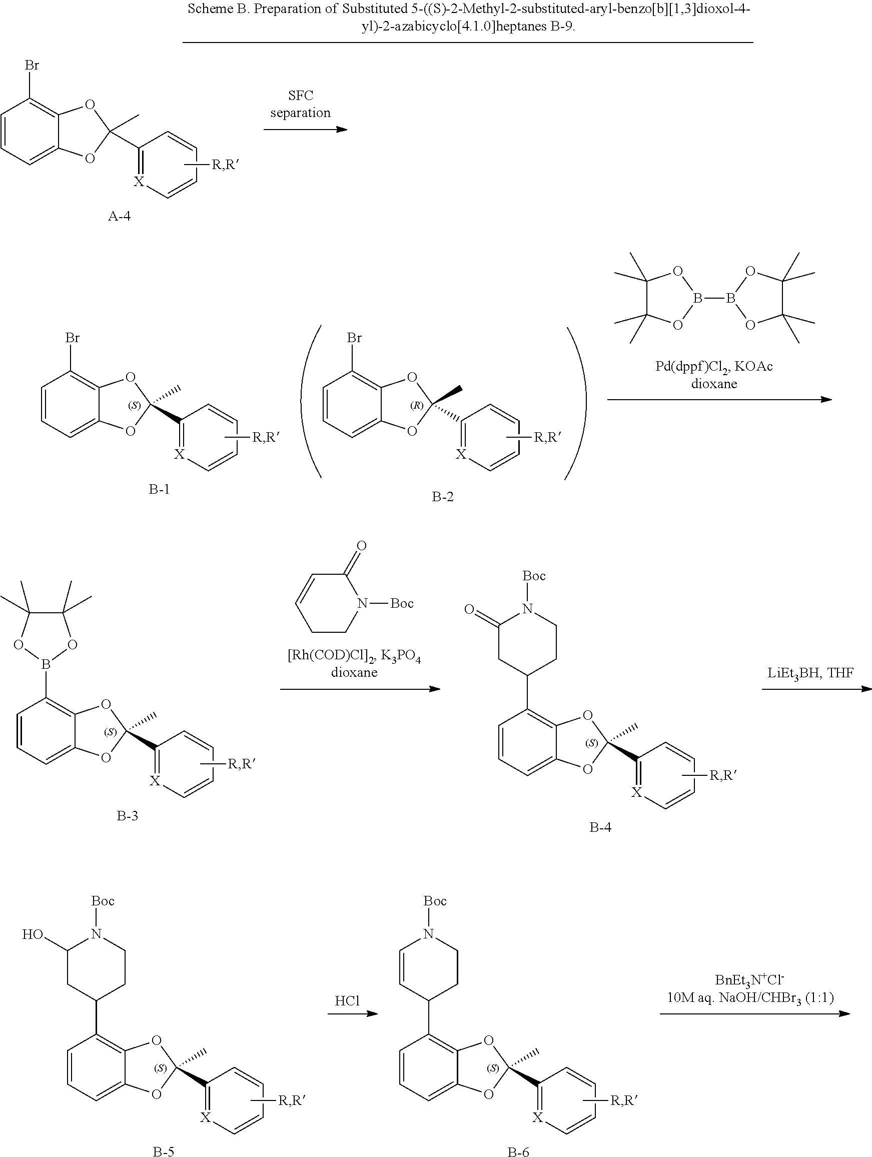

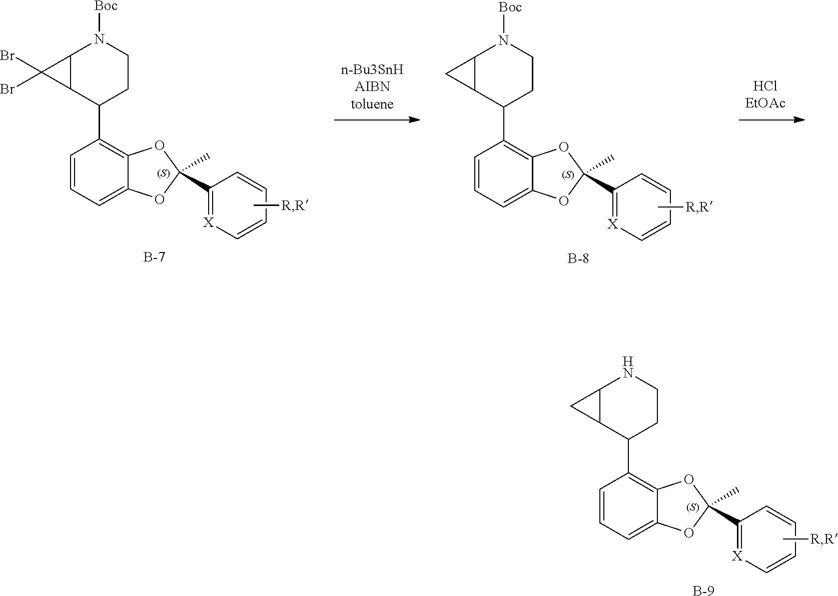

Substituted 4-bromo-2-methyl-2-substituted-aryl-benzo[d][1,3]dioxoles A-4 may be separated by supercritical fluid chromatography (SFC) and the desired S-isomer B-1 converted via Miyaura reaction to the boronate ester B-3. Rhodium catalyzed cross-coupling of B-3 with the commercially available tert-butyl 6-oxo-3,6-dihydropyridine-1(2H)-carboxylate provides lactam B-4. Reduction of lactam B-4 provides hydroxypiperidine B-5 which is treated with aqueous acid to provide dihydropyridine B-6.

Reaction of B-6 with dibromocarbene, generated under phase transfer conditions, provides gem-dibromocyclopropane adduct B-7 which is reductively dehalogenated with tri-n-butyltin hydride. Deprotection of B-8 with strong acid, such as HCl or TFA, provides B-9.

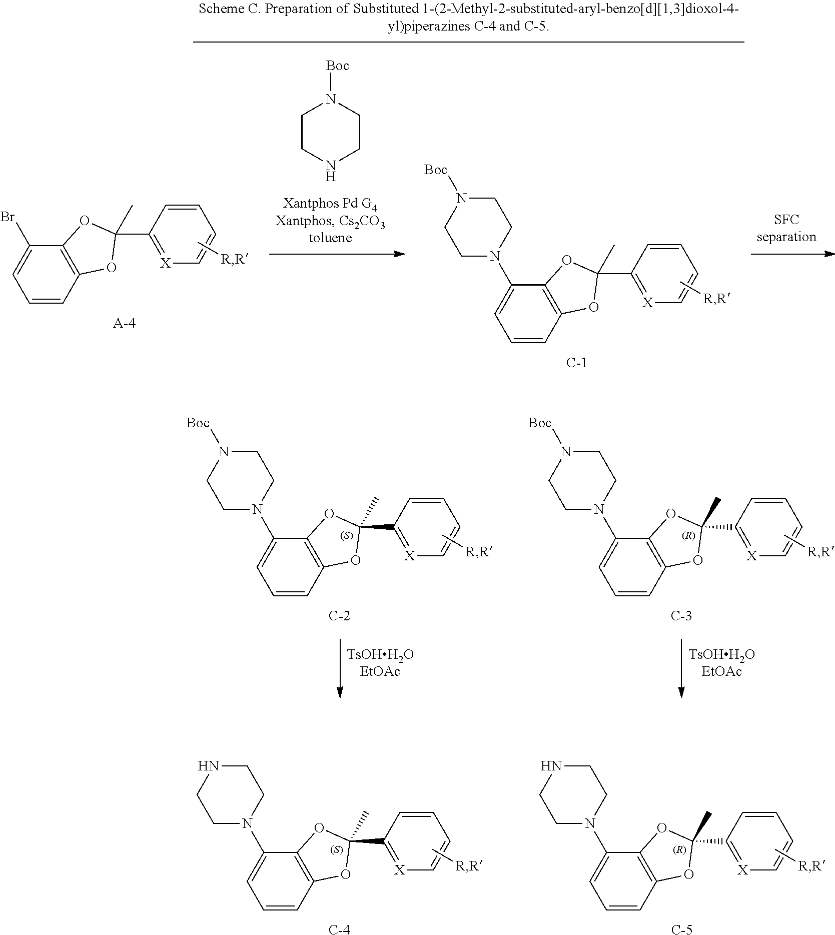

Commercially available tert-butyl piperazine-1-carboxylate is reacted with A-4 under Buchwald amination conditions and the resulting intermediate separated by supercritical fluid chromatography (SFC) and deprotected with strong acid to provide C-4 and C-5.

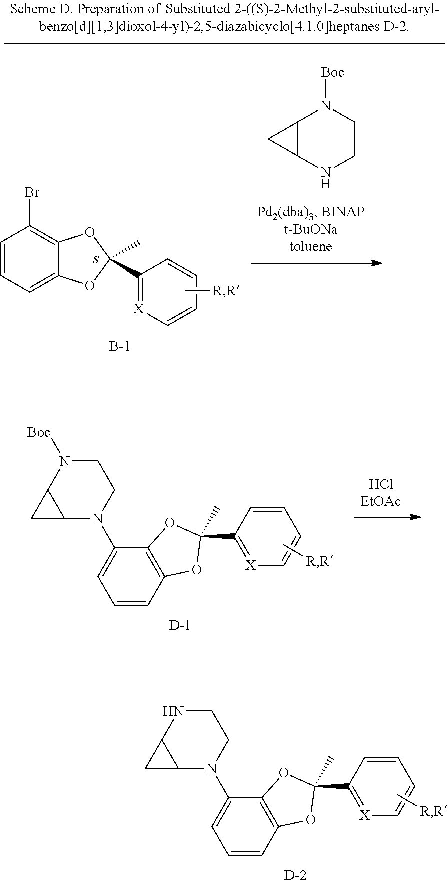

Commercially available tert-butyl 2,5-diazabicyclo[4.1.0]heptane-2-carboxylate is reacted with B-1 under Buchwald amination conditions and the resulting intermediate D-1 deprotected with strong acid to provide D-2.

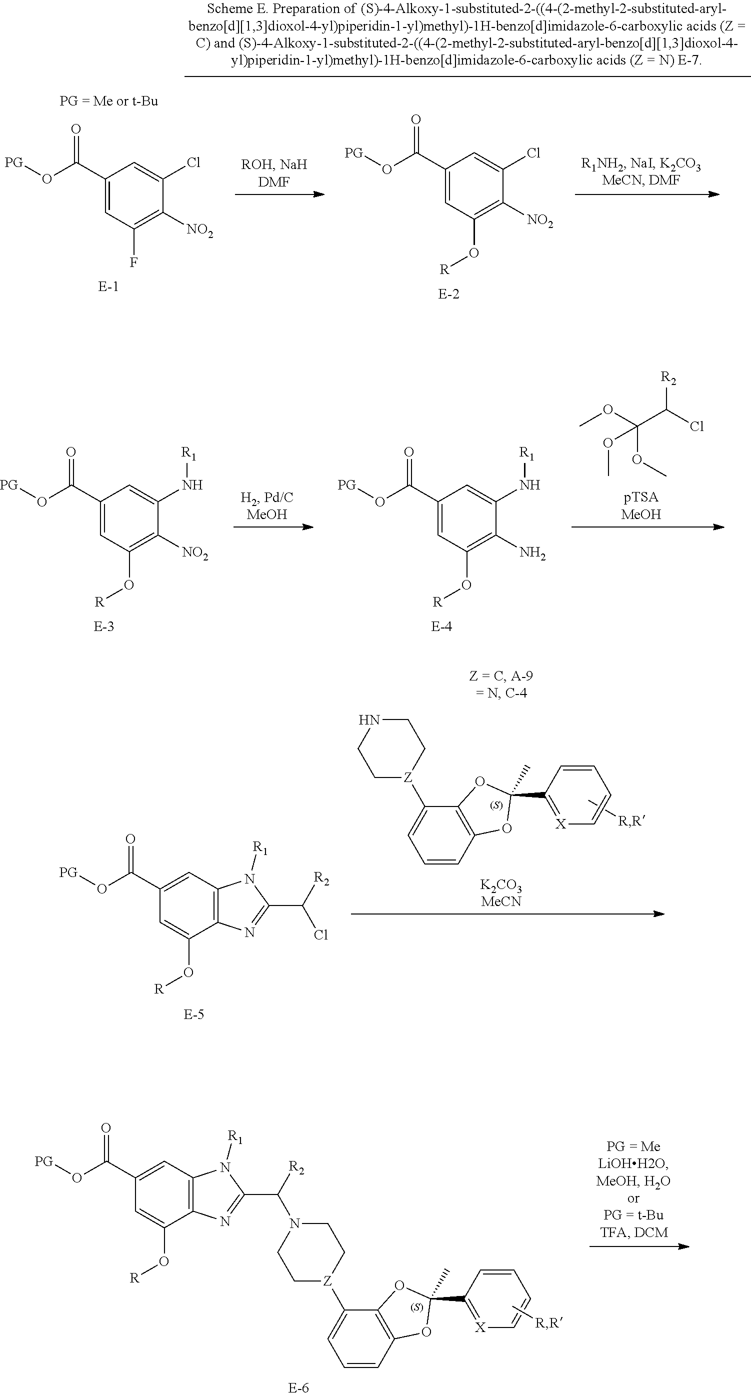

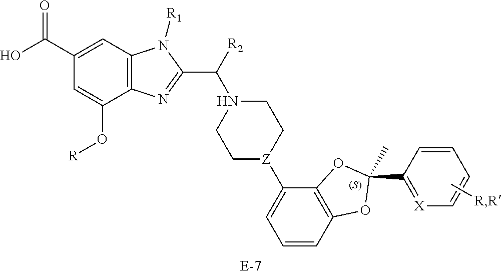

Reaction of methyl or t-butyl 3,5-difluoro-4-nitrobenzoate E-1 with an alkoxide derived from an alcohol and a strong base such as sodium hydride provides E-3. Nucleophilic substitution of E-3 with a desired amine provides E-4 which undergoes cyclization with an optionally substituted 2-chloro-1,1,1-trimethoxyethane to provide 2-(chloromethyl)-1H-benzo[d]imidazole E-5. E-5 and A-9/C-4 are reacted in the presence of an acid scavenger such as an alkali carbonate to provide E-6 which is then deprotected with an alkali hydroxide (PG-Me) or strong acid (PG=tBu) to provide E-7.

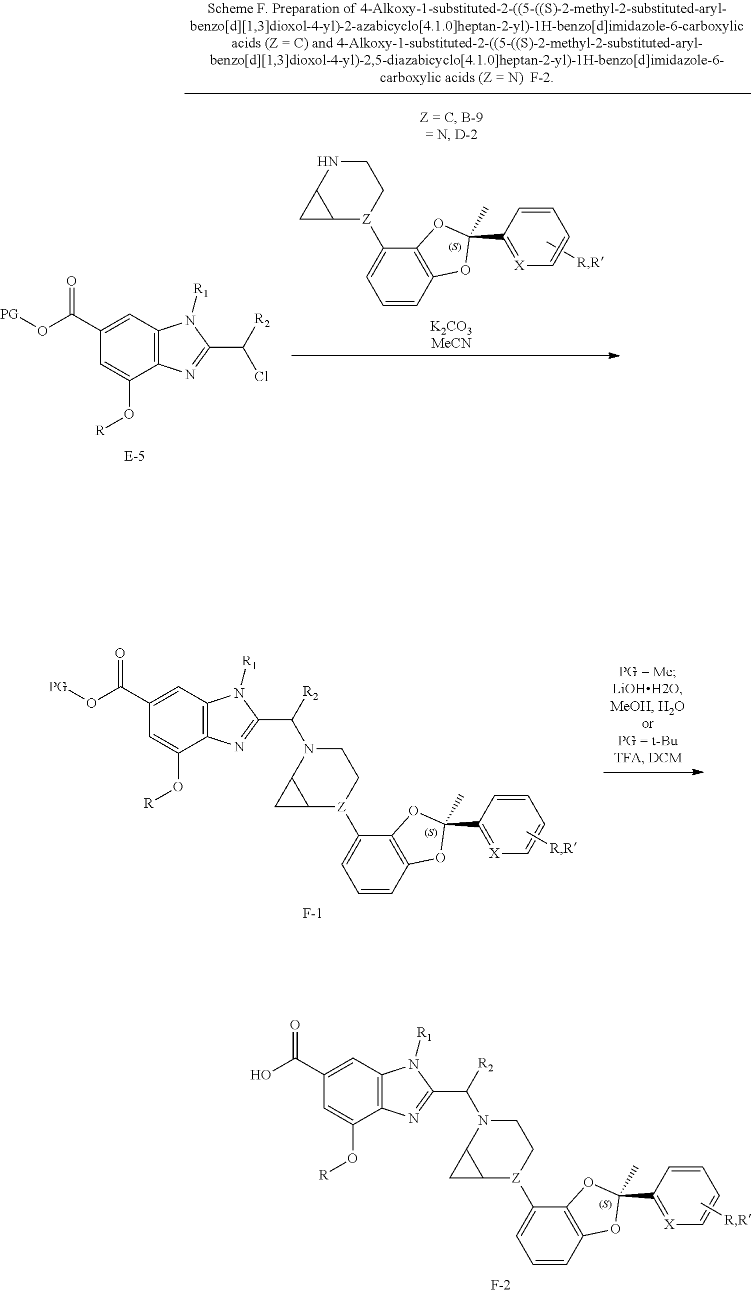

2-(Chloromethyl)-1H-benzo[d]imidazole E-5 and amine B-9/D-2 are reacted in the presence of an acid scavenger such as an alkali carbonate to provide F-1 which is then deprotected with an alkali hydroxide (PG=Me) or strong acid (PG=tBu) to provide F-2.

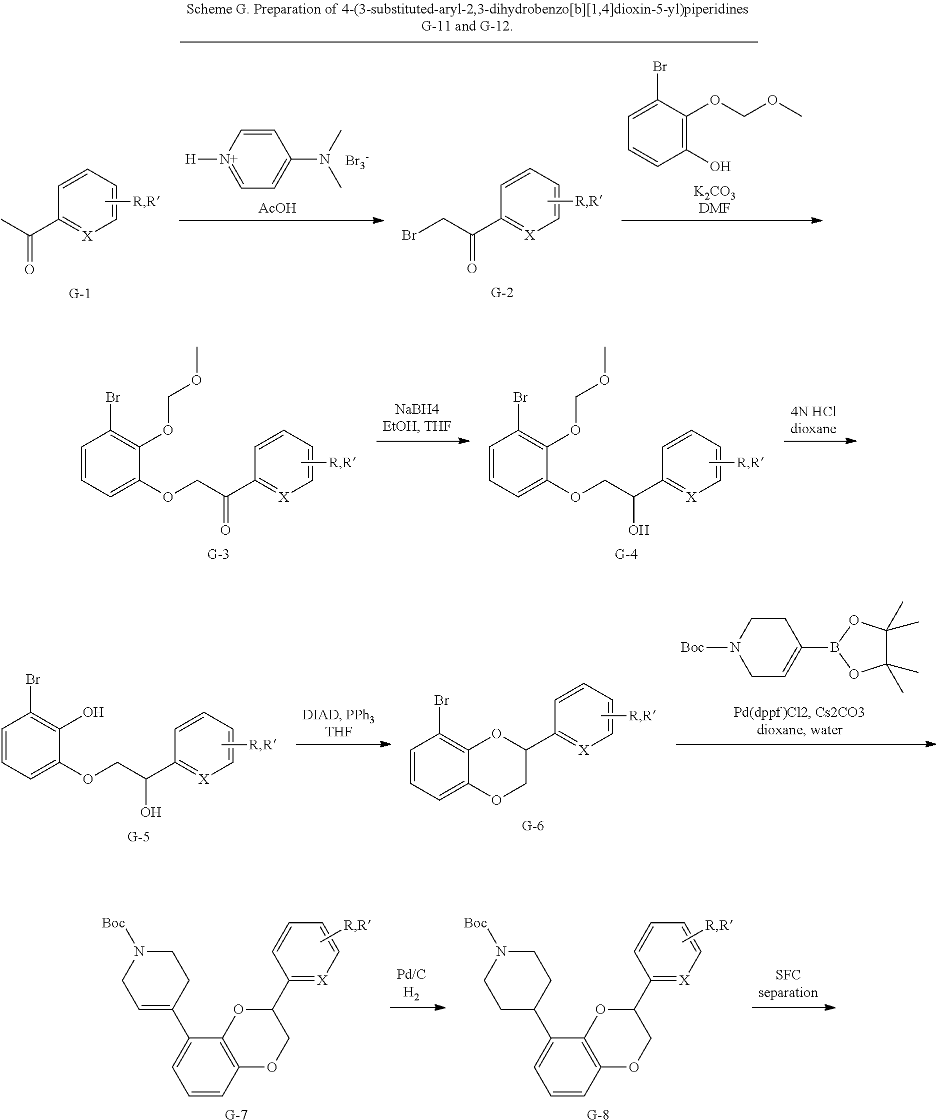

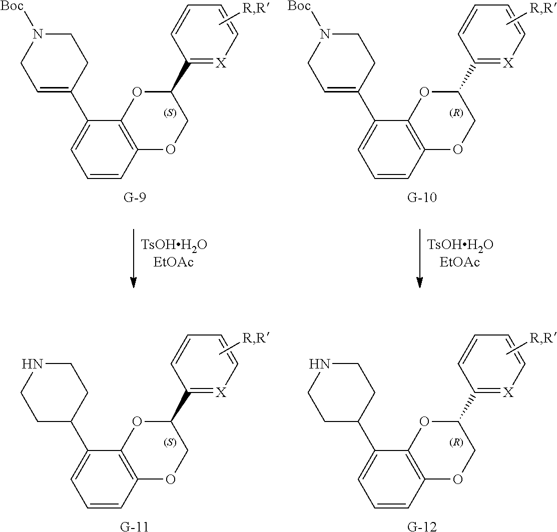

Reaction of 3-bromo-2-(methoxymethoxy) phenol with bromoketone G-2 in the presence of base provides phenoxyketone G-3 which is reduced with a suitable reducing agent such as sodium borohydride. Deprotection and cyclization under Mitsunobu conditions provides 2,3-dihydrobenzo[b][1,4]dioxine G-6. Suzuki coupling of G-6 with commercially available tert-butyl 4-(4, 4, 5, 5-tetramethyl-1,3,2-dioxaborolan-2-yl)-3,6-dihydropyridine-1(2H)-carboxylate followed by reduction of the dihydropyridine intermediate provides G-8 which may be separated by super critical fluid chromatography (SFC). Enantiomers G-9 and G-10 are deprotected with strong acid to provide amines G-11 and G-12.

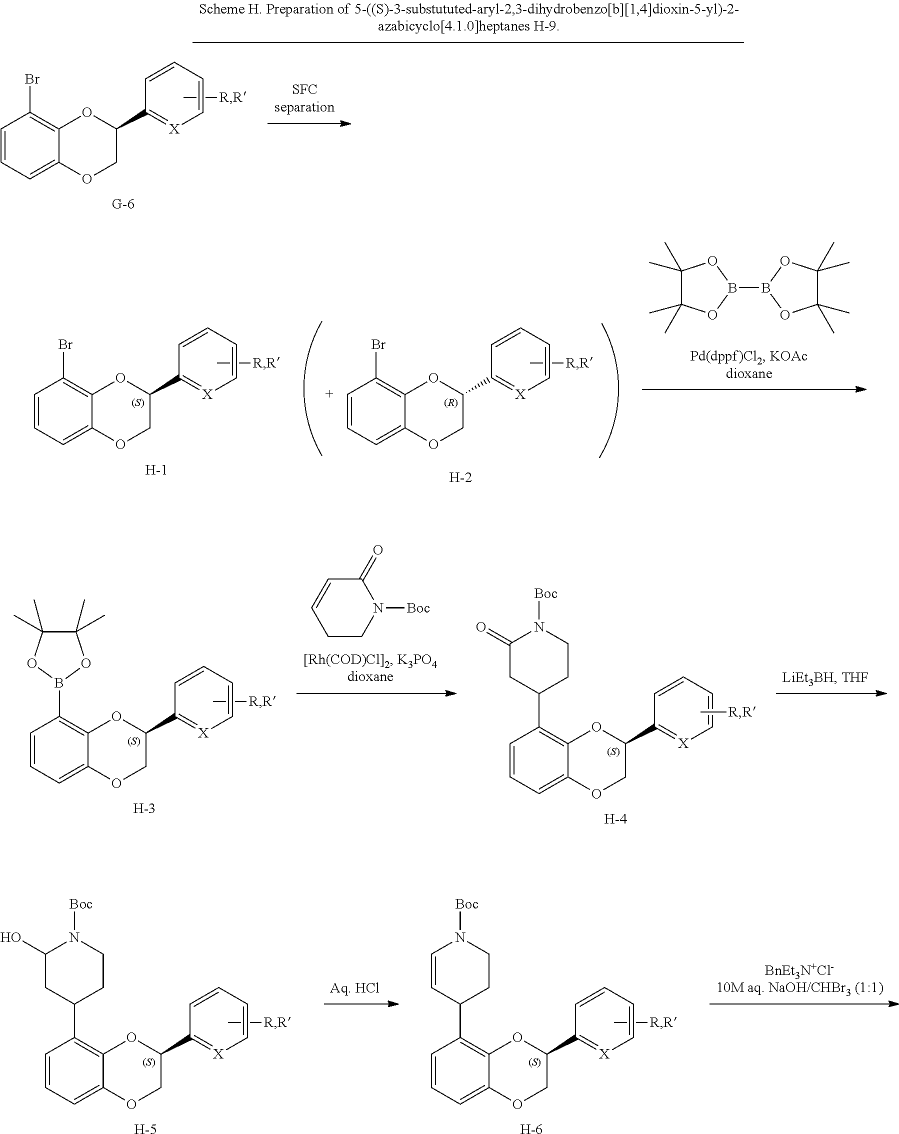

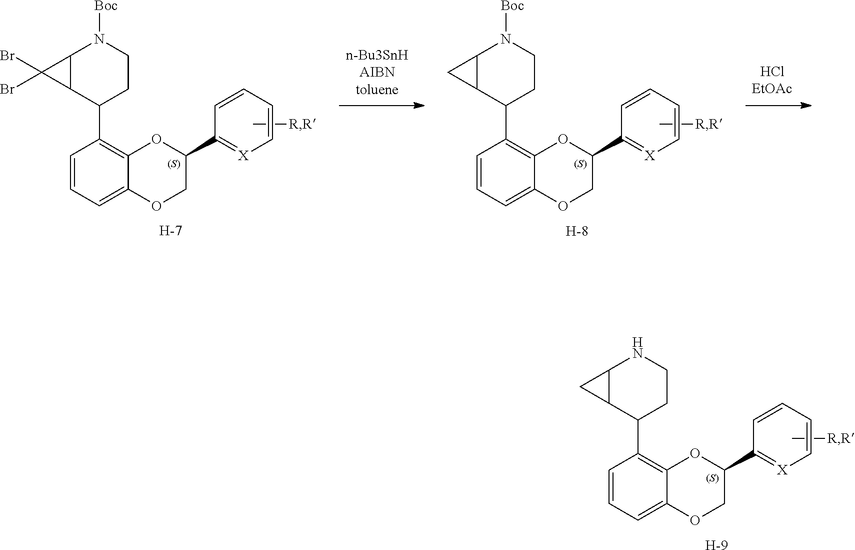

Substituted 8-bromo-2-substituted-aryl-2,3-dihydrobenzo[b][1,4]dioxines G-6 may be separated by supercritical fluid chromatography (SFC) and the desired S-isomer H-1 converted via Miyaura reaction to the boronate ester H-3. Rhodium catalyzed cross-coupling of H-3 with the commercially available tert-butyl 6-oxo-3,6-dihydropyridine-1(2H)-carboxylate provides lactam H-4. Reduction of lactam H-4 provides hydroxypiperidine H-5 which is treated with aqueous acid to provide dihydropyridine H-6.

Reaction of H-6 with dibromocarbene, generated under phase transfer conditions, provides gem-dibromocyclopropane adduct H-7 which is reductively dehalogenated with tri-n-butyltin hydride. Deprotection of H-8 with strong acid, such as HCl or TFA, provides H-9.

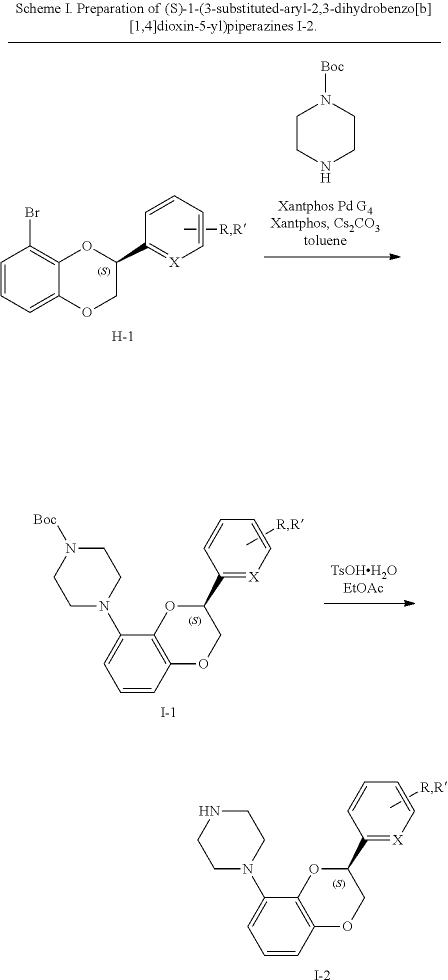

Commercially available tert-butyl piperazine-1-carboxylate is reacted with H-1 under Buchwald amination conditions and the resulting intermediate I-1 deprotected with strong acid to provide I-2.

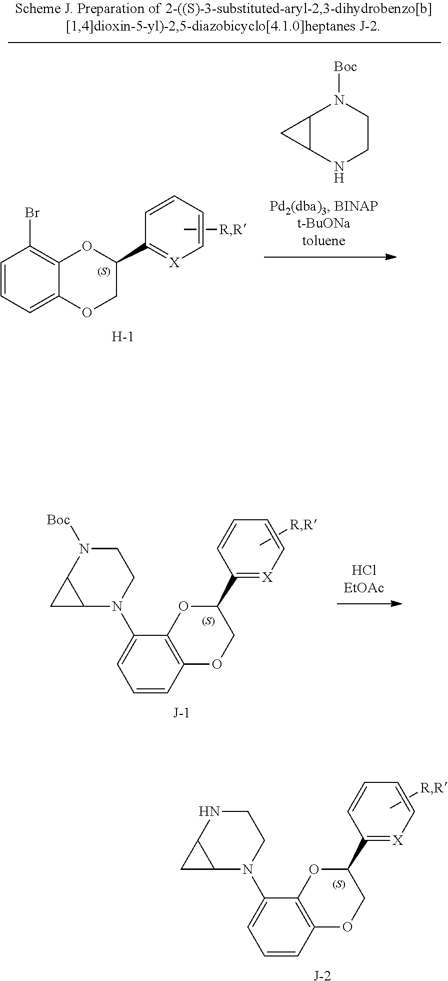

Commercially available tert-butyl 2,5-diazabicyclo[4.1.0]heptane-2-carboxylate is reacted with H-1 under Buchwald amination conditions and the resulting intermediate J-1 deprotected with strong acid to provide J-2.

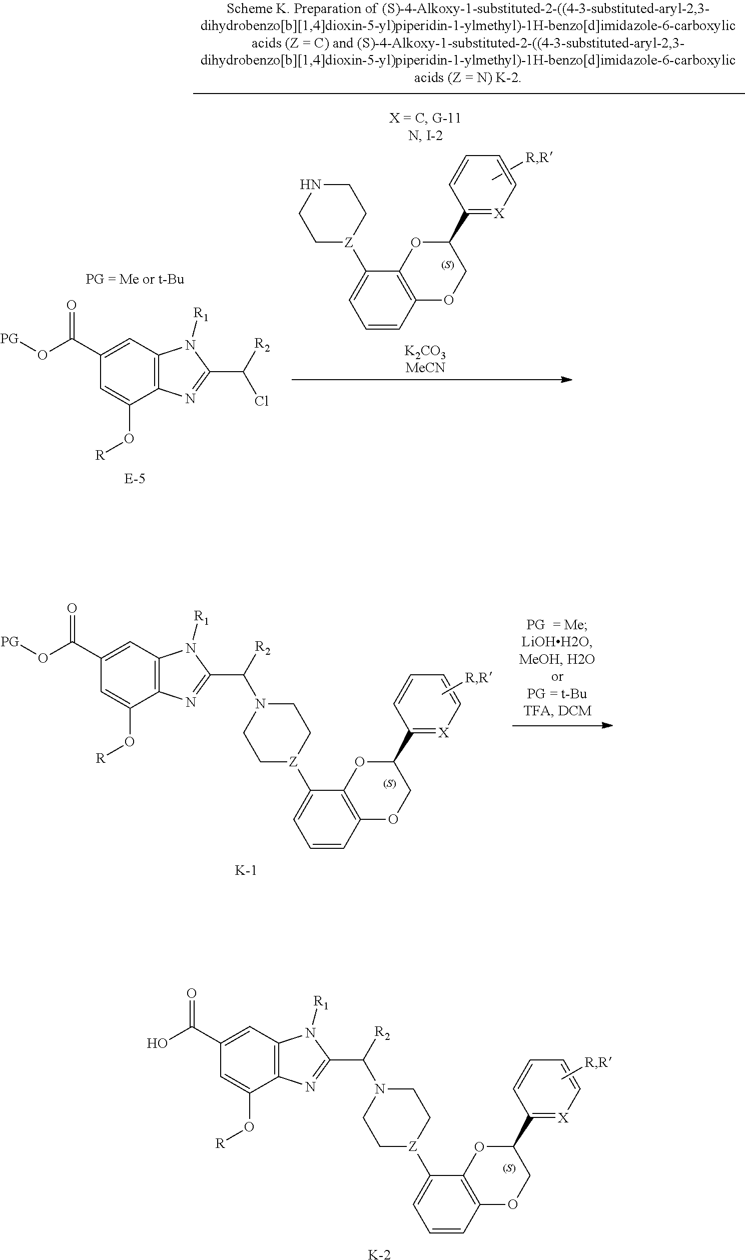

2-(Chloromethyl)-1H-benzo[d]imidazole E-5 and amine G-11/1-2 are reacted in the presence of an acid scavenger such as an alkali carbonate to provide K-1 which is then deprotected with an alkali hydroxide (PG=Me) or strong acid (PG=tBu) to provide K-2.

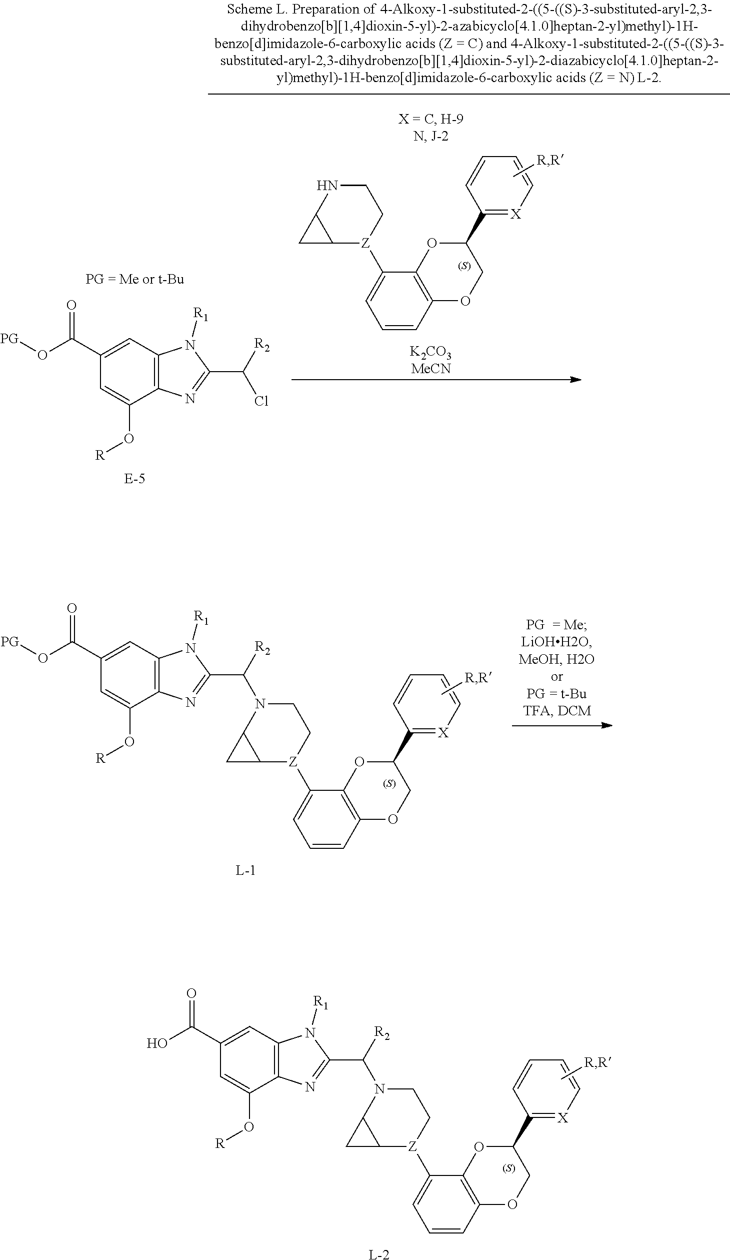

2-(Chloromethyl)-1H-benzo[d]imidazole E-5 and amine H-9/J-2 are reacted in the presence of an acid scavenger such as an alkali carbonate to provide L-1 which is then deprotected with an alkali hydroxide (PG=Me) or strong acid (PG=tBu) to provide L-2.

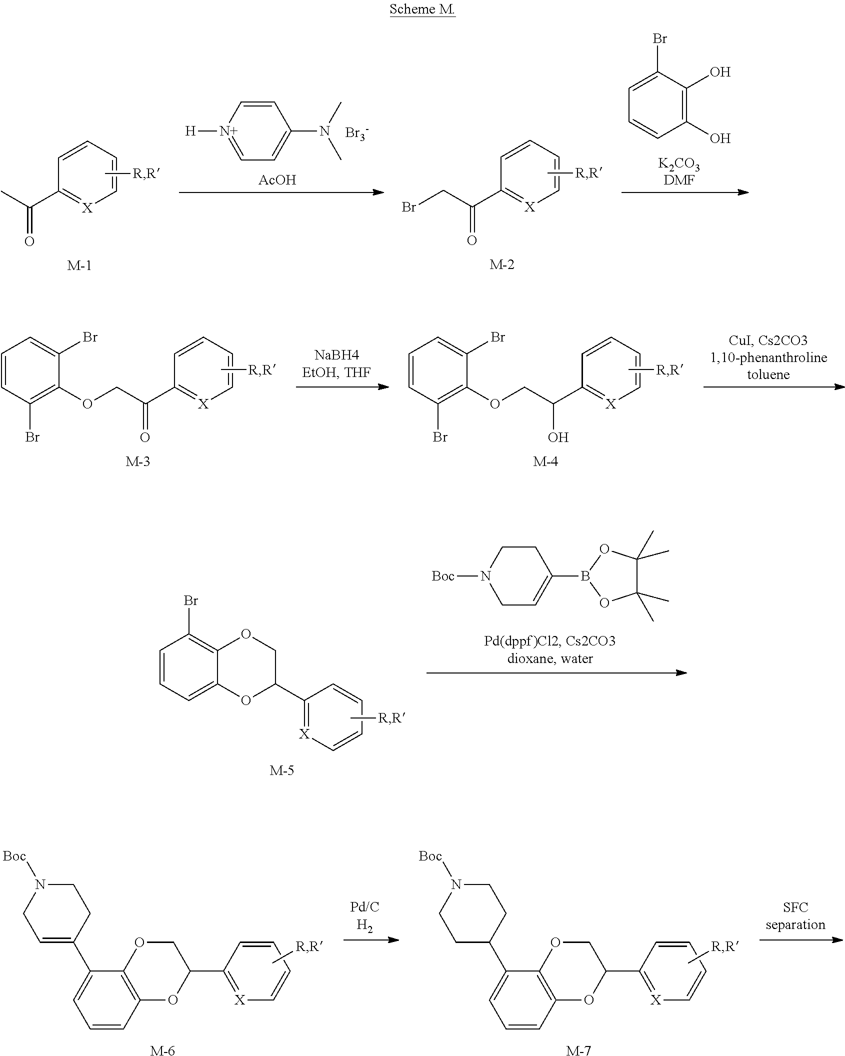

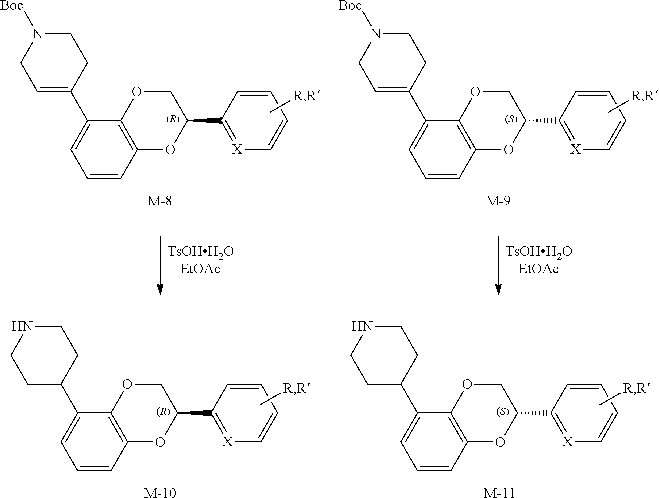

Reaction of 2,6-dibromophenol with bromoketone M-2 in the presence of base provides phenoxyketone M-3 which is reduced with a suitable reducing agent such as sodium borohydride. Copper catalyzed cyclization of M-4 in the presence of 1,10-phenanthroline ligand provides M-5. Suzuki coupling of M-5 with commercially available tert-butyl 4-(4, 4, 5, 5-tetramethyl-1,3,2-dioxaborolan-2-yl)-3,6-dihydropyridine-1(2H)-carboxylate followed by reduction of the dihydropyridine intermediate provides M-7 which may be separated by super critical fluid chromatography (SFC). Enantiomers M-8 and M-9 are deprotected with strong acid to provide amines M10 and M-11.

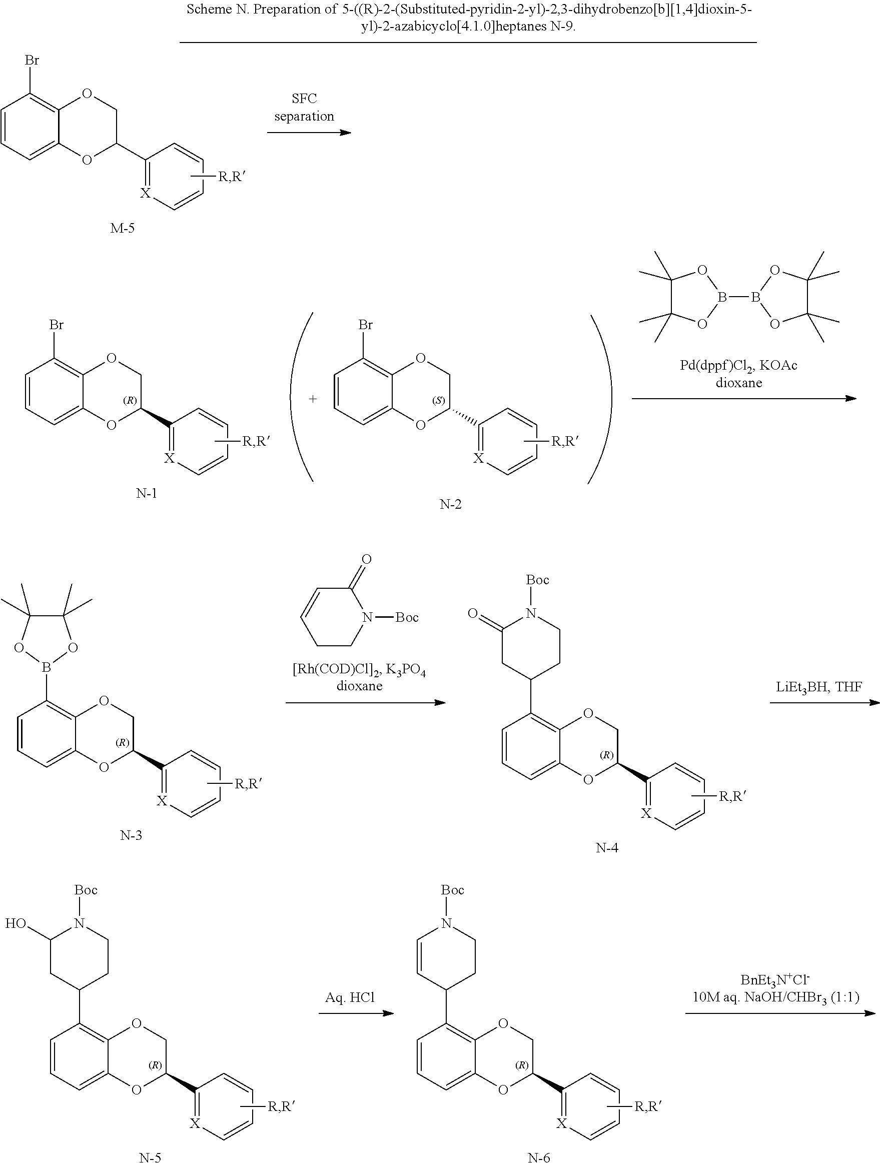

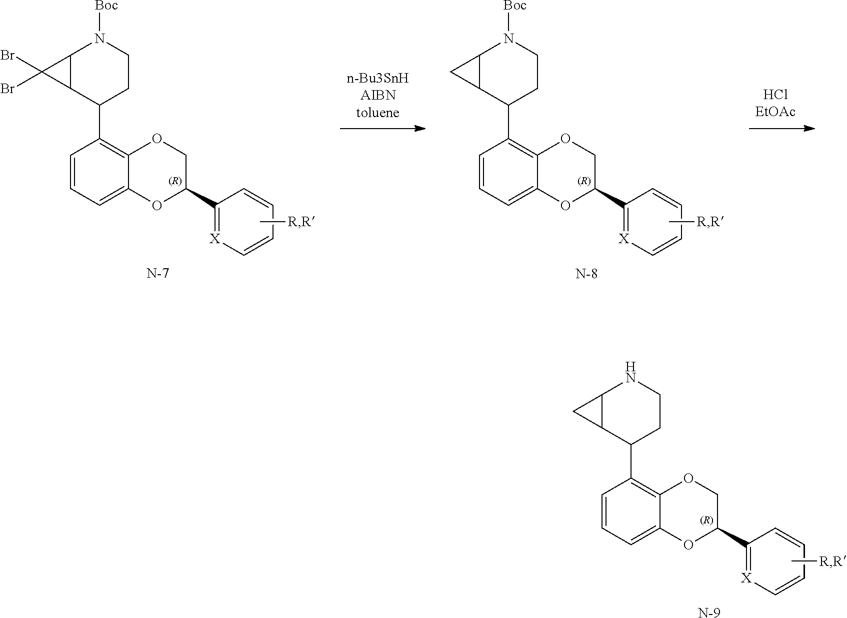

Substituted 5-bromo-2-substituted-aryl-2,3-dihydrobenzo[b][1,4]dioxines M-5 may be separated by supercritical fluid chromatography (SFC) and the desired S-isomer N-1 converted via Miyaura reaction to the boronate ester N-3. Rhodium catalyzed cross-coupling of N-3 with the commercially available tert-butyl 6-oxo-3,6-dihydropyridine-1(2H)-carboxylate provides lactam N-4. Reduction of lactam N-4 provides hydroxypiperidine N-5 which is treated with aqueous acid to provide dihydropyridine N-6.

Reaction of N-6 with dibromocarbene, generated under phase transfer conditions, provides gem-dibromocyclopropane adduct N-7 which is reductively dehalogenated with tri-n-butyltin hydride. Deprotection of N-8 with strong acid, such as HCl or TFA, provides N-9.

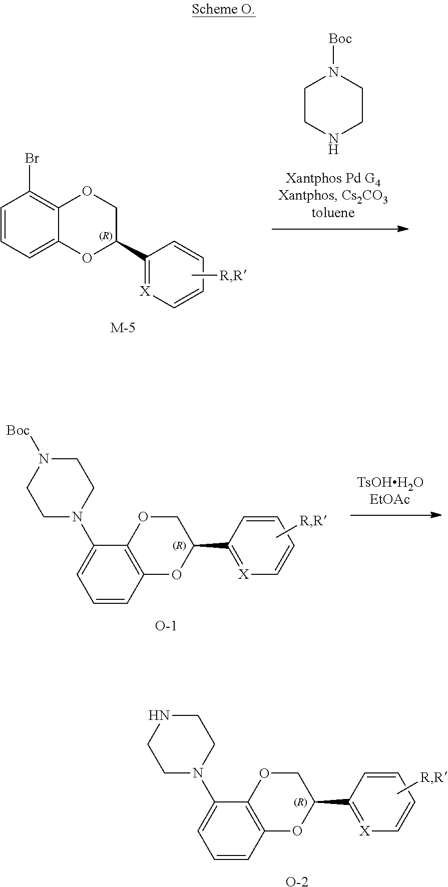

Commercially available tert-butyl piperazine-1-carboxylate is reacted with M-5 under Buchwald amination conditions and the resulting intermediate O-1 deprotected with strong acid to provide O-2.

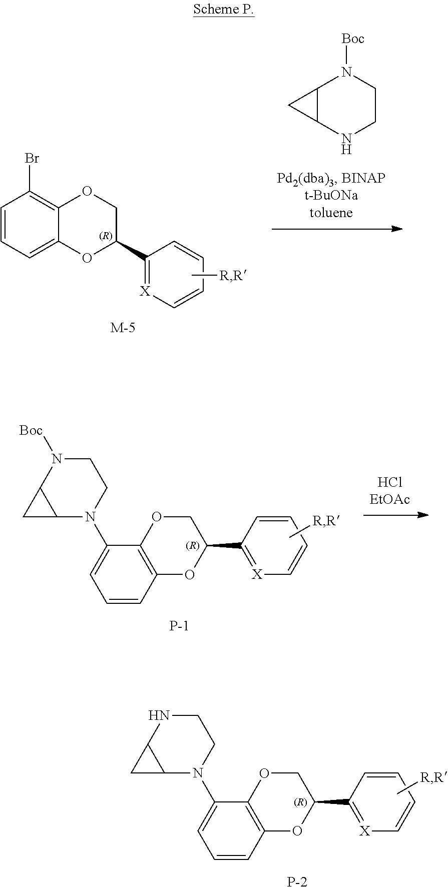

Commercially available tert-butyl 2,5-diazabicyclo[4.1.0]heptane-2-carboxylate is reacted with M-5 under Buchwald amination conditions and the resulting intermediate P-1 deprotected with strong acid to provide P-2.

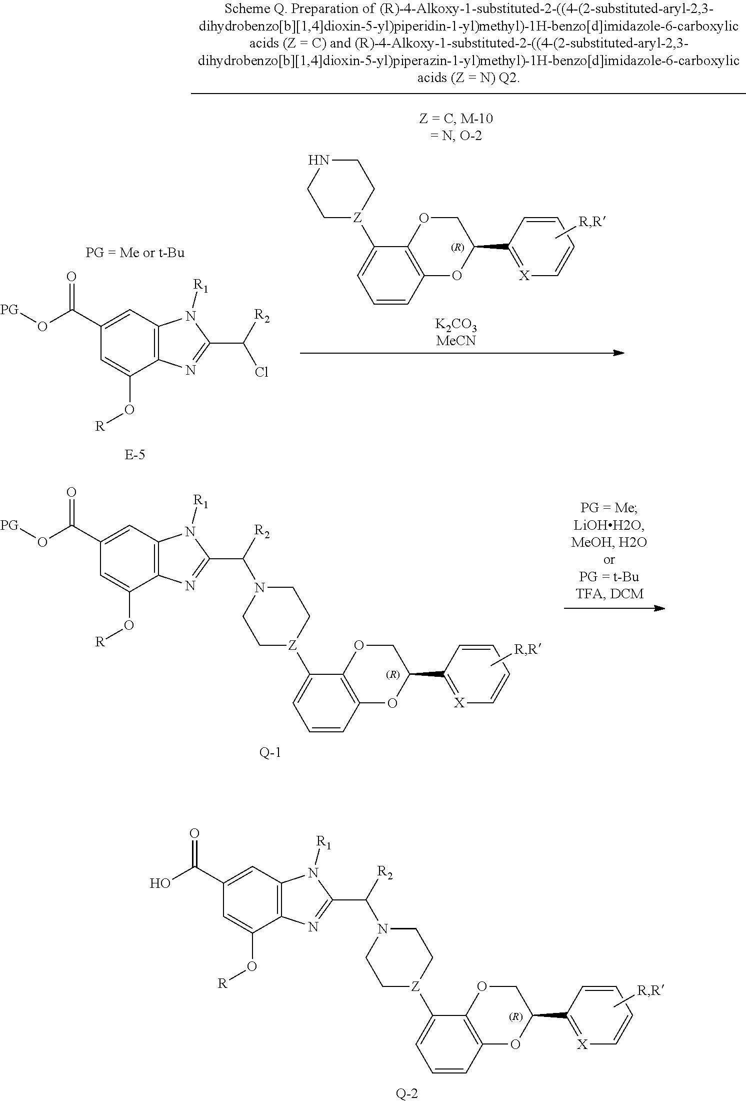

2-(Chloromethyl)-1H-benzo[d]imidazole E-5 and amine M-10/0-2 are reacted in the presence of an acid scavenger such as an alkali carbonate to provide Q-1 which is then deprotected with an alkali hydroxide (PG-Me) or strong acid (PG-tBu) to provide Q-2.

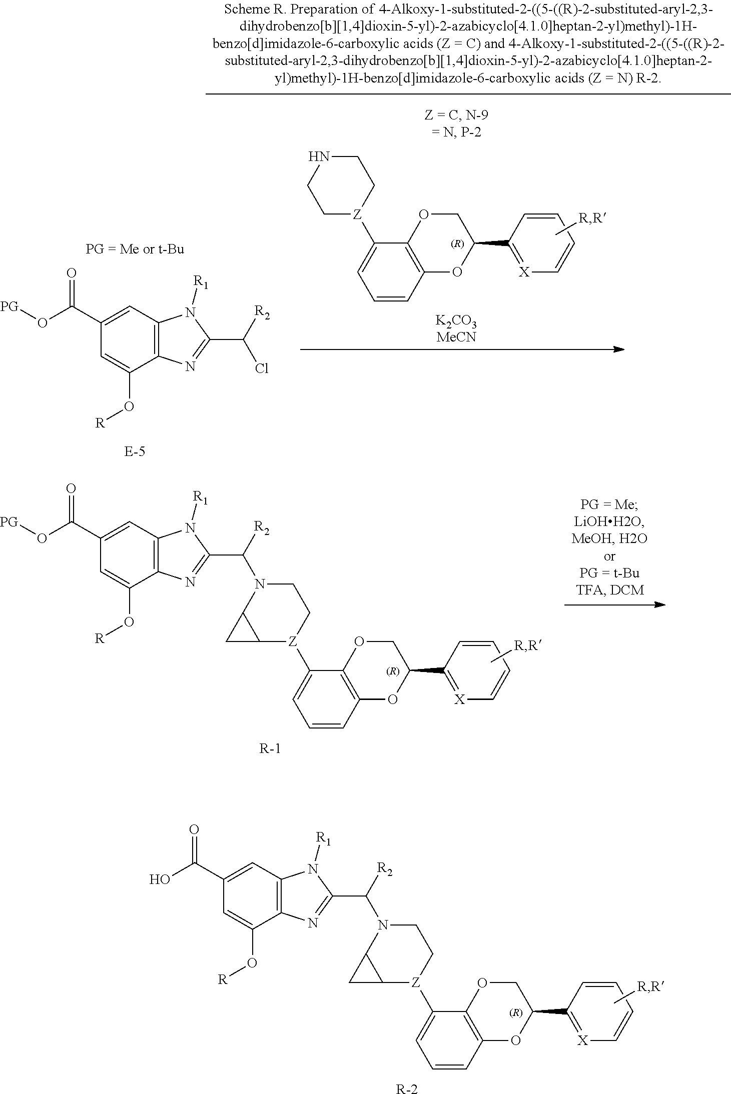

2-(Chloromethyl)-1H-benzo[d]imidazole E-5 and amine N-9/P-2 are reacted in the presence of an acid scavenger such as an alkali carbonate to provide R-1 which is then deprotected with an alkali hydroxide (PG=Me) or strong acid (PG=tBu) to provide R-2.

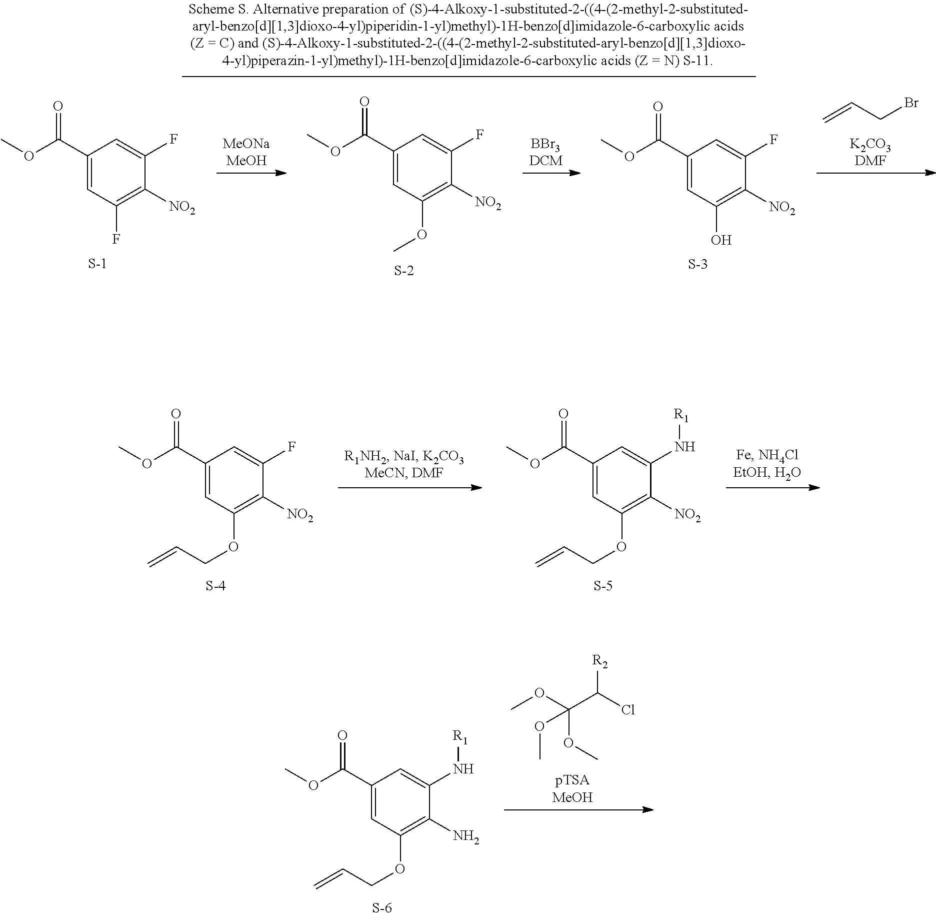

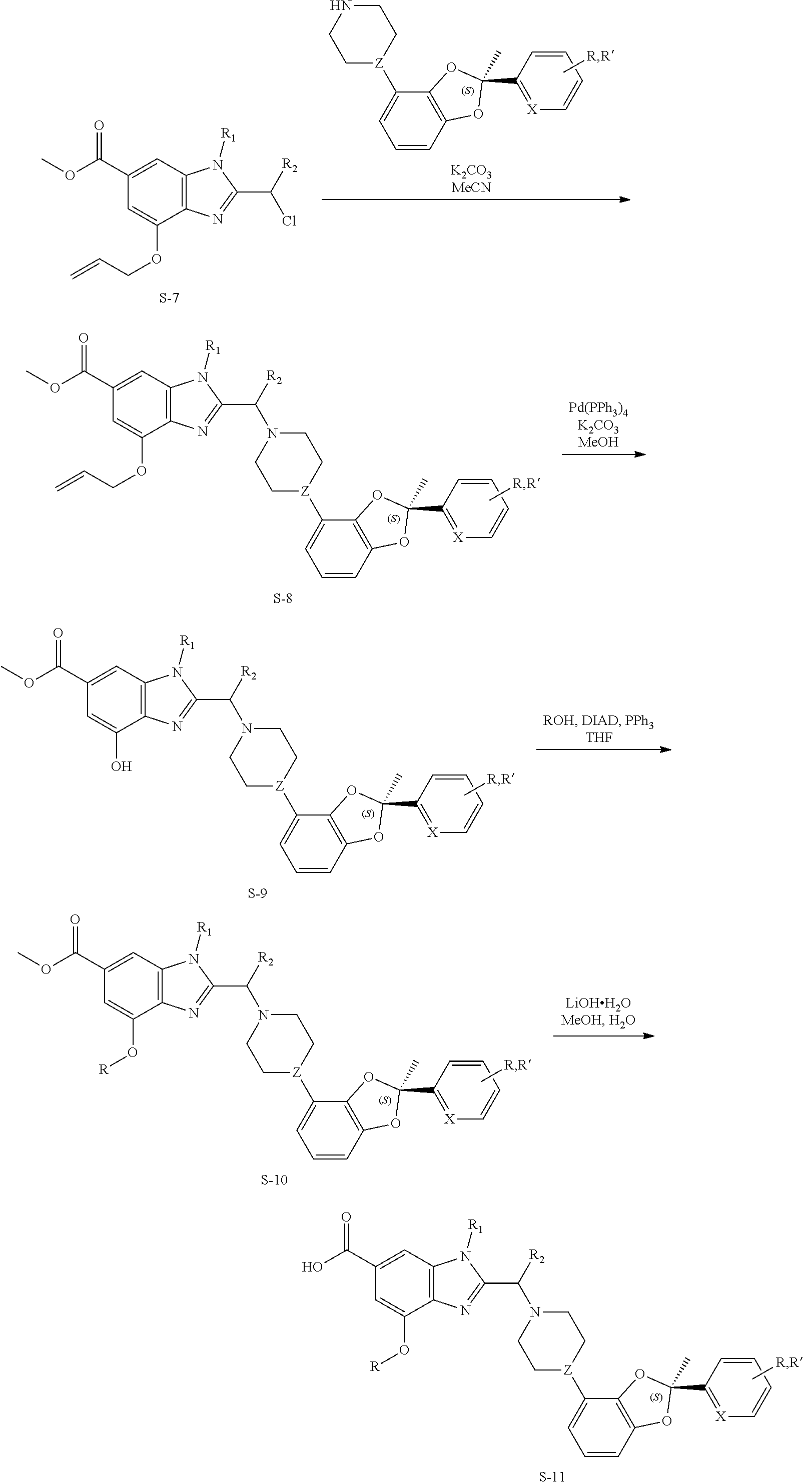

An alternative route to 4-alkoxybenzimidazoles relies on the palladium catalyzed deprotection of allyl protected intermediate S-8 prepared similarly to previous examples. Palladium catalyzed allyl deprotection provides phenol S-9 which is converted to 4-alkoxybenzimidazole S-10 via Mitsunobu alkylation. S-10 is then deprotected with an alkali hydroxide to provide S-11.

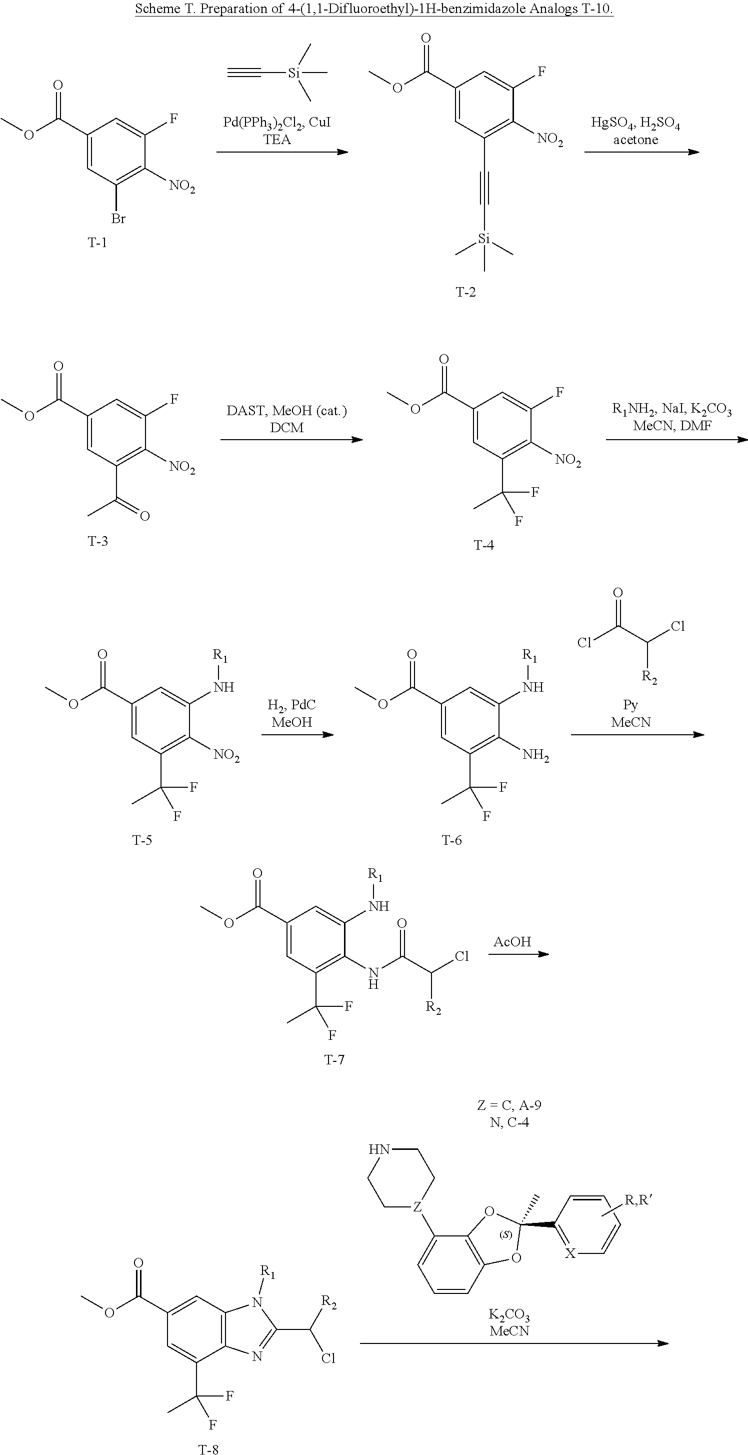

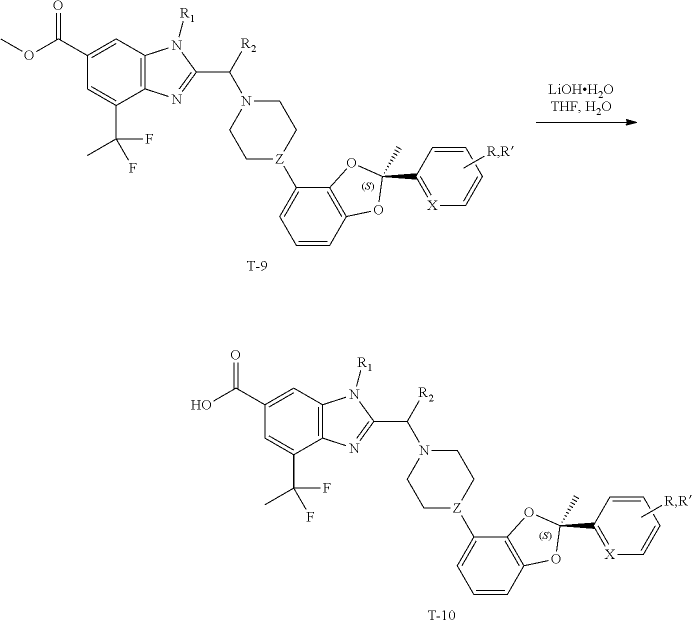

Mercury (II) catalyzed hydration of T-2 derived from Sonogashira coupling of methyl 3-bromo-5-fluoro-4-nitrobenzoate and ethynyltrimethylsilane provides ketone T-3. The key 1,1-difluoroethyl group is introduced via DAST mediated difluorination. Nucleophilic substitution with an appropriate amine and reduction provides diamine T-6. Reaction with an optionally substituted 2-chloroacetyl chloride and cyclization in acetic acid provides 2-(chloromethyl)-benzimidazole T-8. T-8 and amine A-9/C-4 are reacted in the presence of an acid scavenger such as an alkali carbonate to provide T-9 which is then deprotected with an alkali hydroxide to provide T-10.

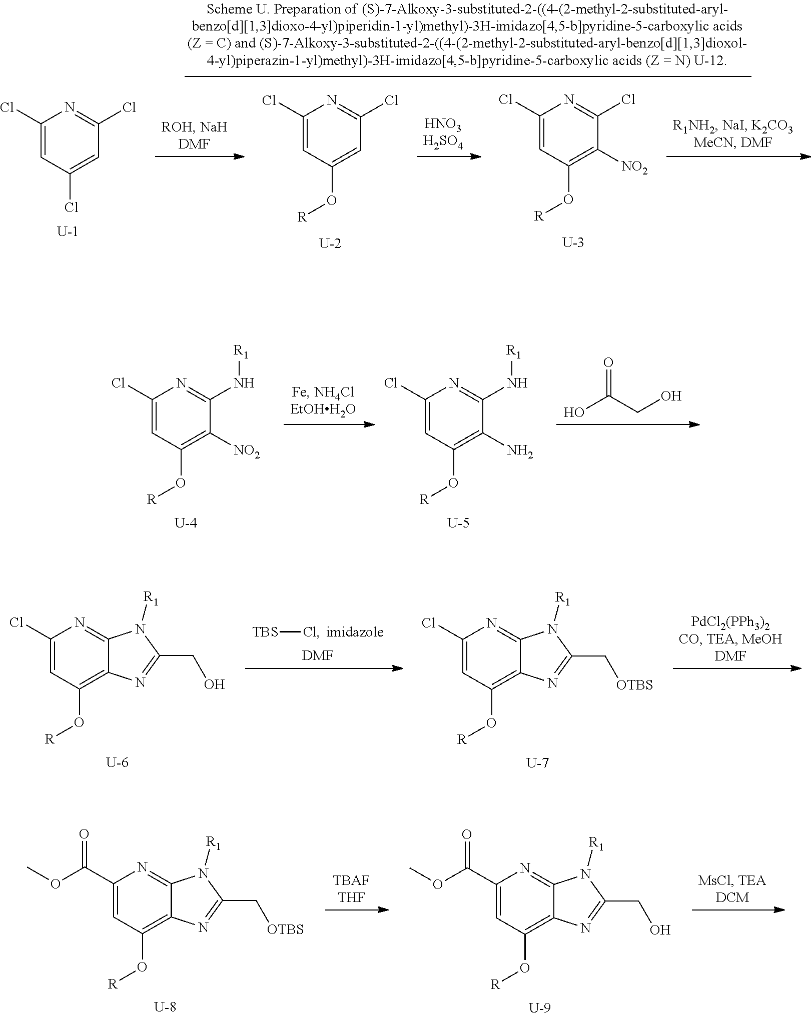

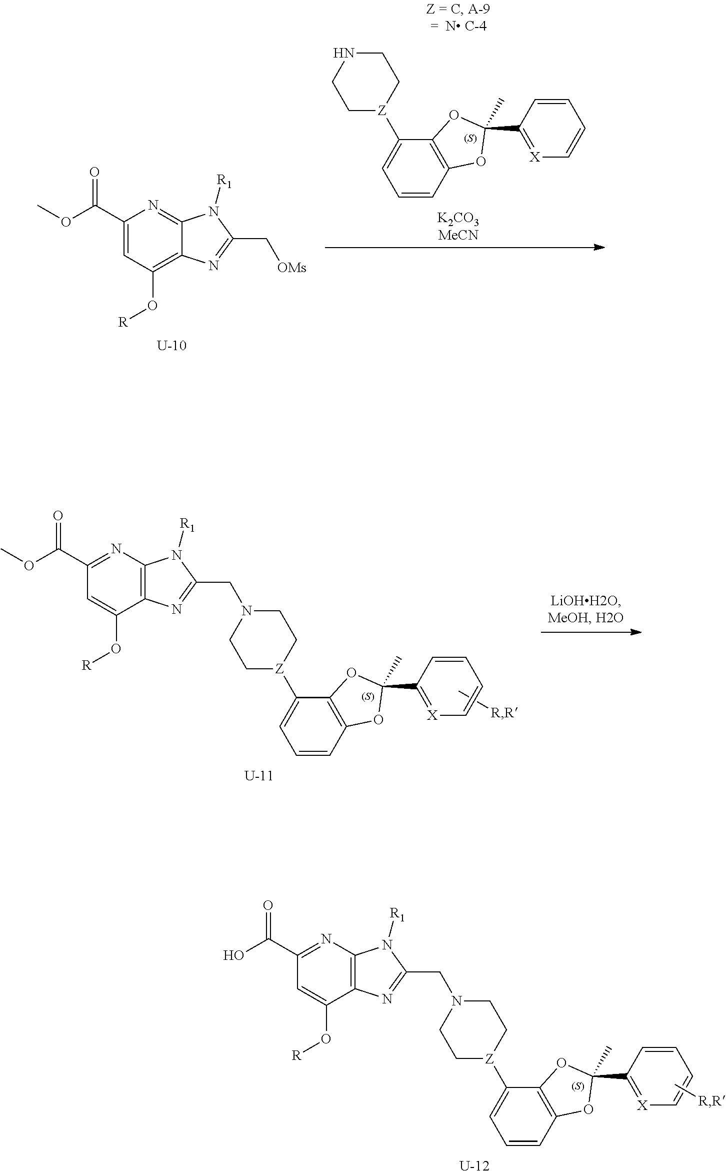

2,6-Dichloro-4-alkoxypyridine U-2 prepared from 2,4,6-trichloropyridine and an alkali alkoxide is nitrated and then reacted with an appropriate amine. Reduction provides diamine U-5 which is cyclized with an optionally substituted glycolic acid and then protected as a silyl ether. Palladium catalyzed carbonylation provides imidazopyridine ester U-8. Silyl group deprotection followed by activation as a mesylate ester provides U-10. U-10 and amine A-9/C-4 are reacted in the presence of an acid scavenger such as an alkali carbonate to provide U-11 which is then deprotected with an alkali hydroxide to provide U-12.

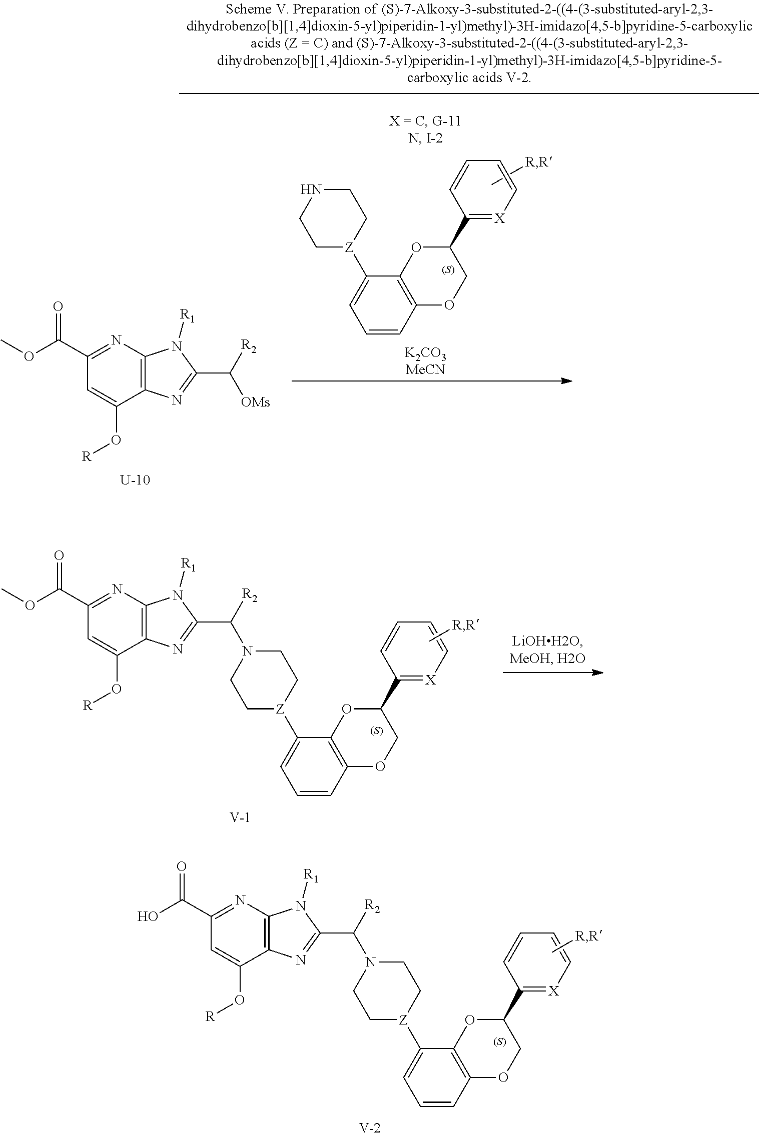

2-(((Methylsulfonyl)oxy)methyl)-3H-imidazo[4,5-b]pyridine U-10 and amine G-11/1-2 are reacted in the presence of an acid scavenger such as an alkali carbonate to provide V-1 which is then deprotected with an alkali hydroxide (PG=Me) or strong acid (PG=tBu) to provide V-2.

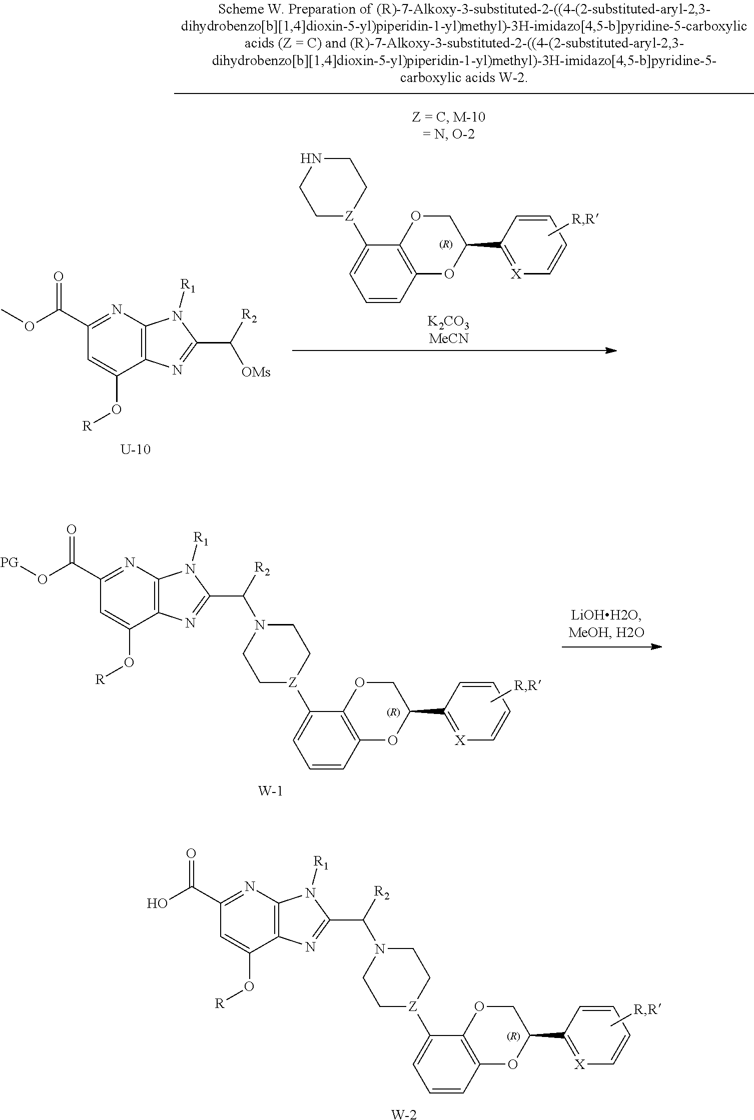

2-(((Methylsulfonyl)oxy)methyl)-3H-imidazo[4,5-b]pyridine U-10 and amine M-10/0-2 are reacted in the presence of an acid scavenger such as an alkali carbonate to provide W-1 which is then deprotected with an alkali hydroxide (PG=Me) or strong acid (PG-tBu) to provide W-2.

### Biological Assays

Compounds designed, selected and/or optimised by methods described above, once produced, can be characterised using a variety of assays known to those skilled in the art to determine whether the compounds have biological activity. For example, the molecules can be characterised by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

Various in vitro or in vivo biological assays are may be suitable for detecting the effect of the compounds of the present disclosure. These in vitro or in vivo biological assays can include, but are not limited to, enzymatic activity assays, electrophoretic mobility shift assays, reporter gene assays, in vitro cell viability assays, and the assays described herein.

### GLP-1R Cell Assay

Stable cell lines expressing high and low GLP-1R surface expression are generated in CHO-K1 cells transfected (Fugene 6) with a puromycin selectable DNA plasmid encoding human

GLP-1R receptor (accession number: NM_002062.5) under control of an EF1A promoter. Transfected cells are seeded into 24-well plates (9,000 cells/well) containing complete medium and incubated in a humidified incubator at 37° C. with 5% carbon dioxide. After overnight incubation, medium is replaced with complete medium supplemented with puromycin (6 μg/mL) and refreshed every 2-3 days to select for stably transfected cells. Individual pools of selected cells are expanded prior to analysis for responsiveness to GLP-1 control peptide using a TR-FRET assay to detect cAMP (LANCE Ultra cAMP Assay, Perkin Elmer). Briefly, cells are collected in Versene solution, plated in 384-well plates (1,000 cells/well) and combined with serially diluted GLP-1R control peptide (10 nL) using an acoustic dispenser (ECHO). Plates are incubated for 30 minutes at 25° C. prior to the addition of EU-CAMP tracer (5 μL) and Ulight-anti-cAMP (5 μL) reagents to each well, followed by 15 minutes incubation at 25° C. TR-FRET signal is detected using an EnVision Multimode Plate Reader (excitation=320 nm; emission=615 and 655 nm). Dose-response curves are used to generate EC₅₀ values as a measure of responsiveness to the GLP-1R control peptide. Selected cell lines are monitored for responsiveness over multiple passages to ensure stability. CHO-K1_hGLP-1Rhigh_clone16 and CHO-K1_hGLP-1Rlow_clone10 showed consistently high and low responsiveness to GLP-1R control peptide, respectively, and are chosen for further analysis to determine relative levels of GLP-1R surface expression. Briefly, GLP-1R expression is analyzed by flow cytometry using a fluorescein-labeled Exendin-4 peptide fluorescent probe (FLEX). Cells are harvested in Versene solution and washed 3-times with PBS+0.5% BSA before incubation with FLEX reagent (10 μM) for 2 hours at room temperature. After incubation, cells are washed 3-times in PBS+0.5% BSA before final resuspension in PBS prior to analysis by flow cytometry to measure FLEX mean fluorescence intensity (MFI) as a measure of GLP-1R expression on the cell surface.

For compound testing in the CHO-K1_hGLP-1Rlow_clone10 cell lines, cells are seeded in 384-well plates (1,000 cells/well). Test compounds are serially diluted in DMSO (10-point, 3-fold dilution), added to wells using an ECHO dispenser (10 nL/well) and plates are centrifuged for 1 min and agitated for 2 min at room temperature prior to 30-minute incubation at 25° C. After incubation, Eu-CAMP (5 L) and Ulight-anti-cAMP (5 μL) reagents are added to each well, followed by centrifugation for 1 minute, agitation for 2 minutes at room temperature, and final incubation of the plates at 25° C. for 15 minutes. Plates are read using an EnVision microplate reader (excitation=320 nm; emission=615 and 655 nm). Dose-response curves are generated from duplicate wells based on percent activation calculated relative to a control GLP-1 peptide agonist that was run in parallel. EC₅₀ values are determined by fitting percent activation as a function of compound concentration using the Hill equation (XLfit).

### Metabolic Stability in Hepatocytes

Test compounds are incubated in human hepatocytes and stability is assessed from the substrate depilation approach. Test compounds are dissolved in dimethyl sulfoxide (DMSO) to create a 10 mM Stock, and then further diluted to create a 1000× Working Stock of 1 mM with DMSO in 96-well plates for test compounds and the positive control (midazolam). Vials containing cryopreserved hepatocytes are removed from the liquid nitrogen tank and immediately immersed in a 37° C. water bath. The vials are shaken gently until the contents had thawed and were then immediately emptied into 48 mL of pre-warmed HT Medium in a 50 ml conical tube. Cells remaining in the vial are resuspended with 1.0 mL of pre-warmed HT Medium and added to the conical tube. The tube is capped and then gently inverted several times to resuspend the hepatocytes. The cell suspension is centrifuged at 50×g at room temperature for 5 minutes and the supernatant discarded. The cell pellet is loosened by gently swirling the centrifuge tube and is re-suspended in 4 mL of warm Dulbecco's Modified Eagle medium (DMEM). Cell density is determined by a cell counter by Nexcelom, and DMEM medium is added to obtain a target density of 1×106 cells/mL. The assay is carried out in 96-well microtiter plates. Test Compounds are incubated at 1 μM with 1×10⁶ cells/mL hepatocytes in DMEM for 0, 30, 60, 120 and 240 minutes. The incubation is carried out with gentle shaking at 37° C. under a humid atmosphere of 95% air/5% CO₂. The volume of the incubation mixture is 37 μL with a final 0.1% DMSO. At each of the time points, the incubation is stopped by adding 150 μL quenching solution (100% acetonitrile, 0.1% formic acid containing bucetin as an internal standard for positive ESI mode). Subsequently, the mixtures are vortexed for 20 min and centrifuged at 4,000 RPM at 10° C. The supernatant (80 μL) is transferred to a clean 96-well plate and analyzed by LC-MS/MS. Midazolam at 1 μM with a final 0.1% DMSO is included as a positive control to verify assay performance. The percent parent remaining, intrinsic and predicted hepatic clearance and t_{1/2} are calculated. All samples are analyzed by LC-MS/MS using an AB Sciex API 4000 instrument, coupled to a Shimadzu LC-20AD LC Pump system. Separation is achieved using a Waters Atlantis T3 dC18 reverse phase HPLC column (20 mm×2.1 mm) at a flow rate of 0.5 mL/min. The mobile phase consists of 0.1% formic acid in water (solvent A) and 0.1% formic acid in 100% acetonitrile (solvent B). Elution conditions are detailed below.

| Time | Flow | % | % |
|---|---|---|---|
| (min) | (μL/min) | A | B |
| 0 | 500 | 98 | 2 |
| 0.30 | 500 | 98 | 2 |
| 1.40 | 500 | 2 | 98 |
| 2.20 | 500 | 2 | 98 |
| 2.21 | 500 | 98 | 2 |
| 3.00 | 500 | 98 | 2 |

The ion optics of each test compound are optimized for their declustering potential (DP), collection energy (CE), collision-cell exit potential (CXP) and used in a selected ion monitoring experiment in the positive ion mode. The peak area ratio of each test compound to internal standard is then evaluated for stability. The extent of metabolism is calculated based on the disappearance of the test compound, compared to its initial concentration. The initial rates of clearance of the test compound are calculated using the linear regression plot of semi-log % remaining of the compound versus time. The elimination rate constant (k) of the linear regression plot is then used to determine t_{1/2} and the intrinsic clearance (CLᵢₙₜ) using the following formula, where C_{hepatocyte} (million cells/mL) is the cell density of the incubation:

$$k = - slope$$

$${t}_{1 / 2} = 0.6 \; 93 / k$$

$${CL}_{int} = k / {C}_{hepatocyte}$$

This method of intrinsic clearance determination assumes that the test compound concentration is far below the Michaelis-Menten constant of the compound to its metabolizing enzymes.

The predicted hepatic clearance (CLₕₑₚ) was calculated using the well stirred method with the following formula with CL_{int(in vivo)} normalized based on liver weight:

$${CL}_{int ( in vivo )} = {CL}_{int} \times Hepatocellularity \times liver weight$$

$${CL}_{hep predicted} = ( {CL}_{int ( in vivo )} \times {Q}_{liver} ) / ( {CL}_{int ( in vivo )} + {Q}_{liver} )$$

Where Qₗᵢᵥₑᵣ ((ml/min/kg) is Liver Blood Flow

The relevant physiological parameters of liver weight, blood flow, and hepatocellularity for humans are listed below:

| Liver Weight | Hepatocellularity | Liver Blood |
|---|---|---|
| (g liver/kg | (106 cells/g | Flow (Qₗᵢᵥₑᵣ, |
| body weight) | liver) | mL/min/kg |
| 25.7 | 135 | 20.7 |

### Passive Permeability and Efflux Ratio

Caco-2 cells (clone C2BBe1) are obtained, e.g., from American Type Culture Collection (Manassas, VA). Cell monolayers are grown to confluence on collagen-coated, microporous membranes in 12-well assay plates. The permeability assay buffer is Hanks' balanced salt solution containing 10 mM HEPES and 15 mM glucose at a pH of 7.4. The buffer in the receiver chamber also contains 1% bovine serum albumin. The dosing solution concentration is 5 μM of test article in the assay buffer. Cell monolayers are dosed on the apical side (A-to-B) or basolateral side (B-to-A) and incubated at 37° C. with 5% CO₂ in a humidified incubator. Samples are taken from the donor and receiver chambers at 120 minutes. Each determination is performed in duplicate. The flux of lucifer yellow is also measured post-experimentally for each monolayer to ensure no damage is inflicted to the cell monolayers during the flux period. All samples are assayed by LC-MS/MS using electrospray ionization. The apparent permeability (Papp) and percent recovery were calculated as follows:

$${P}_{app} = ( {dC}_{r} / dt ) \times {V}_{r} / ( A \times {C}_{A} ) \qquad ( 1 )$$

$$Percent Recovery = 100 \times ( ( {V}_{r} \times {C}_{r}^{final} ) + ( {V}_{d} \times {C}_{d}^{final} ) ) / ( {V}_{d} \times {C}_{N} ) , \qquad ( 2 )$$

where, dCᵣ/dt is the slope of the cumulative receiver concentration versus time in μM s⁻¹; Vᵣ is the volume of the receiver compartment in cm³; V_{d} is the volume of the donor compartment in cm³; A is the area of the insert (1.13 cm² for 12-well); C_{A} is the average of the nominal dosing concentration and the measured 120-minute donor concentration in μM; C_{N} is the nominal concentration of the dosing solution in μM; Cᵣ^{final} is the cumulative receiver concentration in μM at the end of the incubation period; C_{d}^{final} is the concentration of the donor in μM at the end of the incubation period. Efflux ratio (ER) is defined as Pₐₚₚ (B-to-A)/Pₐₚₚ (A-to-B).

### Rat Pharmacokinetics

Intravenous dosing: Compounds are formulated at 10. mg/mL in a solution comprising 5% polyethylene glycol 400 and 95% (12% (w/v) sulfobutyl-β-cyclodextrin in water) (v/v). Formulated compounds are sterile filtered through a 0.22 micron filter before dosing. Compounds are administered to male, 7-11-week-old Sprague-Dawley rats by jugular vein cannula infusion over 30 minutes at a dose of 1 mg/kg.

Oral dosing: Compounds are formulated at 1.0 mg/mL in a solution comprising 5% polyethylene glycol 400 and 95% (12% (w/v) sulfobutyl-β-cyclodextrin in water) (v/v). Formulated compounds are administered to male, 7-11 week old Sprague-Dawley rats by oral gavage at a dose of 10 mL/kg or 3 mL/kg.

Sample collection: Blood collections of about 0.2 mL per time point are performed from jugular vein or other suitable site of each animal, into pre-chilled commercial EDTA-K2 tubes and placed on wet ice until centrifugation. Blood samples are processed for plasma by centrifugation at approximately 4° C., 3,200 g for 10 min. Plasma is collected and transferred into pre-labeled 96 well plate or polypropylene tubes, quick frozen over dry ice and kept at −60° C. or lower until LC-MS/MS analysis.

Data analysis: Plasma concentration versus time data are plotted in graph and analyzed by non-compartmental approaches using the Phoenix WinNonlin 6.3 software program. Related PK parameters are calculated according to dosing route, e.g., CL, Vass and Co for intravenous administration, Cₘₐₓ, Tₘₐₓ or % F for extravascular administration, and T_{1/2}, AUC₍₀₋ₜ₎, AUC_{(0-inf)}, MRT₍₀₋ₜ₎, MRT_{(0-inf)} for all routes.

In some embodiments, the biological assay is described in the Examples herein.

### EXAMPLES

For exemplary purpose, neutral compounds of Formula (I) are synthesized and tested in the examples. It is understood that the neutral compounds of Formula (I) may be converted to the corresponding pharmaceutically acceptable salts of the compounds using techniques in the art (e.g., by saponification of an ester to the carboxylic acid salt, or by hydrolyzing an amide to form a corresponding carboxylic acid and then converting the carboxylic acid to a carboxylic acid salt).

### Abbreviations:

ACN Acetonitrile
AIBN Azobisisobutyronitrile
BOC tert-butyl carbamate
BOP (benzotriazol-1-yloxy)tris(dimethylamino)phosphonium hexafluorophosphate
BTC bis(trichloromethyl) carbonate
CDI carbonyl diimidazole
DAD diode array detector
DCM Dichloromethane
DIEA/DIP
EA N,N-diisopropylethylamine
DMF N,N-dimethylformamide
DMSO Dimethylsulfoxide
EA ethyl acetate
EDCI 1-ethyl-3-(3-dimethylaminopropyl) carbodiimide
ELSD evaporative light scattering detector
ES/ESI electrospray ionisation
HATU 1-[Bis(dimethylamino)methylene]-1H-1,2,3-triazolo[4,5-b]pyridinium 3-oxid hexafluorophosphate
HOAT 1-hydroxy-7-azabenzotriazole
HOBT hydroxy benzotriazole
HPLC high-performance liquid chromatography
IPA Isopropylalcohol
LC liquid chromatography
LiHMDS lithium hexamethyl disilazide
MS mass spectrometry
NMR nuclear magnetic resonance
Py Pyridine
RT retention time
SFC supercritical fluid chromatography
TBAI tetrabutyl ammonium iodide
TEA Triethylamine
TFA trifluoroacetic acid
TFAA trifluoroacetic anhydride
THF Tetrahydrofuran
TLC thin layer chromatography
TMS tetramethyl silane
UV Ultraviolet

### EXAMPLES

For exemplary purpose, neutral compounds of Formula (I) are synthesized and tested in the examples. It is understood that the neutral compounds of Formula (I) may be converted to the corresponding pharmaceutically acceptable salts of the compounds using techniques in the art (e.g., by saponification of an ester to the carboxylic acid salt, or by hydrolyzing an amide to form a corresponding carboxylic acid and then converting the carboxylic acid to a carboxylic acid salt).

### Abbreviations:

ACN Acetonitrile
AIBN Azobisisobutyronitrile
BOC tert-butyl carbamate
BOP (benzotriazol-1-yloxy)tris(dimethylamino)phosphonium BOP hexafluorophosphate
BTC bis(trichloromethyl) carbonate
CDI carbonyl diimidazole
DAD diode array detector
DCM Dichloromethane
DIEA/DIP
EA N,N-diisopropylethylamine
DMF N,N-dimethylformamide
DMSO Dimethylsulfoxide
EA ethyl acetate
EDCI 1-ethyl-3-(3-dimethylaminopropyl) carbodiimide
ELSD evaporative light scattering detector
ES/ESI electrospray ionisation
HATU 1-[Bis(dimethylamino)methylene]-1H-1,2,3-triazolo[4,5-b]pyridinium 3-oxid hexafluorophosphate
HOAT 1-hydroxy-7-azabenzotriazole
HOBT hydroxy benzotriazole
HPLC high-performance liquid chromatography
IPA Isopropylalcohol
LC liquid chromatography
LiHMDS lithium hexamethyl disilazide
MS mass spectrometry
NMR nuclear magnetic resonance
Py Pyridine
RT retention time
SFC supercritical fluid chromatography
TBAI tetrabutyl ammonium iodide
TEA Triethylamine
TFA trifluoroacetic acid
TFAA trifluoroacetic anhydride
THF Tetrahydrofuran
TLC thin layer chromatography
TMS tetramethyl silane
UV Ultraviolet

### Example A1. —Preparation of Compound 1. 2-((4-(6-((4-Cyano-2-fluorobenzyl)oxy)pyridin-2-yl)piperidin-1-yl)methyl)-4-methoxy-1-(thiazol-5-ylmethyl)-1H-benzo[d]imidazole-6-carboxylic acid (Compound 1) (7)

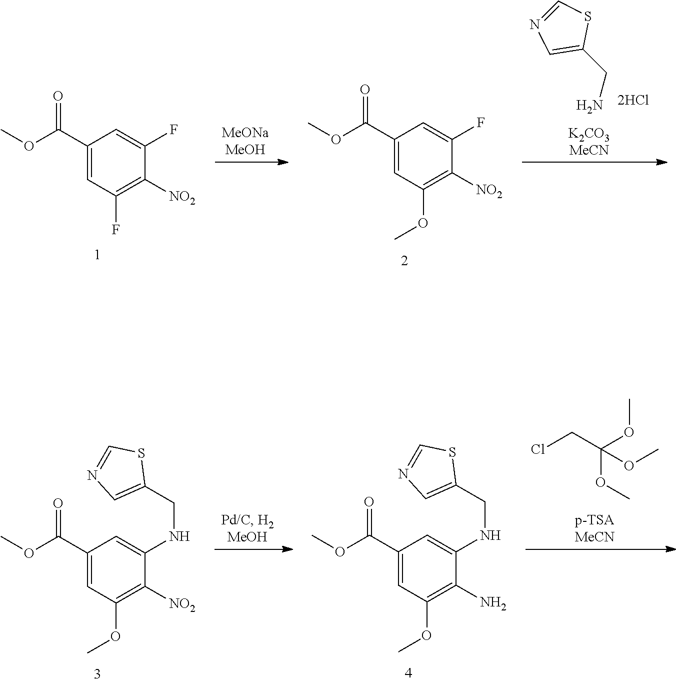

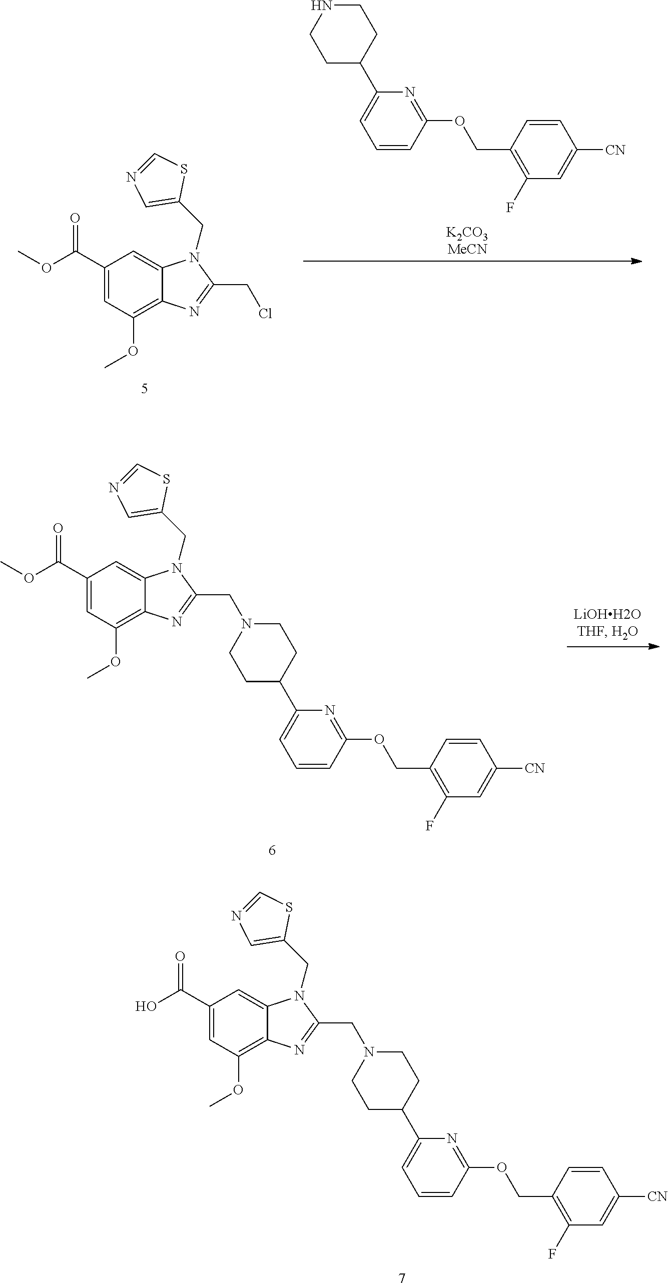

### Preparation of Intermediate

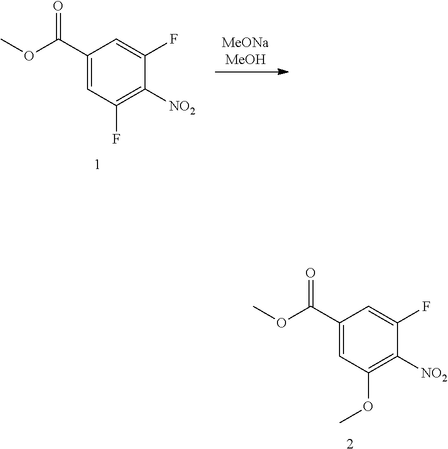

To a solution of methyl 3,5-difluoro-4-nitrobenzoate (1) (2 g, 9.21 mmol, 1 eq) in MeOH (20 mL) was added sodium methoxide (497.62 mg, 9.21 mmol, 1 eq). The mixture was stirred at 0° C. for 2 h. TLC indicated some starting material remained, and one major new spot with stronger polarity was detected. The reaction mixture was concentrated under reduced pressure to remove solvent. The residue was purified by column chromatography (SiO₂, Petroleum ether/Ethyl acetate=10/1 to 3/1) (Rf=0.20) to provide product as a yellow solid (0.7 g, 3.05 mmol, 33% yield).

¹H NMR (400 MHz, CHLOROFORM-d) δ=7.54-7.49 (m, 2H), 4.00 (s, 3H), 3.98 (s, 3H)

### Preparation of Intermediate (3), Methyl 3-methoxy-4-nitro-5-((thiazol-5-ylmethyl)amino)benzoate

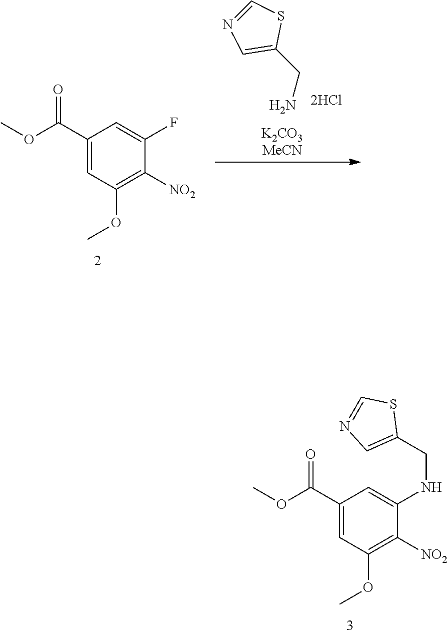

To a mixture of methyl 3-fluoro-5-methoxy-4-nitrobenzoate, 2 (500 mg, 2.18 mmol, 1 eq) and thiazol-5-ylmethanamine hydrochloride (408 mg, 2.18 mmol, 2 eq, 2 HCl) in CH₃CN (5 mL) was added K₂CO₃ (905 mg, 6.55 mmol, 3 eq). The mixture was stirred at 60° C. for 12 h. LC-MS showed starting material was consumed completely and desired mass was detected. The mixture was diluted with H₂O (30 mL), extracted with EtOAc (30 mL*3). The combined organic layers were dried over Na₂SO₄, filtered, and the filtrate was concentrated to give a crude product. The residue was purified by column chromatography (SiO₂, Petroleum ether/Ethyl acetate=100/1 to 1/1) to provide product as a yellow solid (149 mg, 461 μmol, 21% yield).

LCMS: RT=1.769 min, MS cal.: 323.32, [M+H]⁺=325.2

### Preparation of Intermediate (4), Methyl 4-amino-3-methoxy-5-((thiazol-5-ylmethyl)amino)benzoate

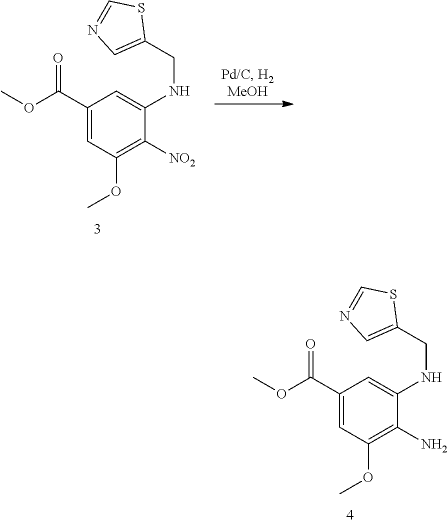

To a solution of methyl 3-methoxy-4-nitro-5-((thiazol-5-ylmethyl)amino)benzoate, 3 (100 mg, 2.12 μmol, 1 eq) in THF (1 mL) was added Pd/C (100 mg, 94 μmol, 10% on carbon) under N₂. The suspension was degassed under vacuum and purged with H₂ several times. The mixture was stirred under H₂ (15 psi) at 20° C. for 1 h. LC-MS showed starting material was consumed completely and the desired mass was detected. The suspension was filtered and the filter cake was washed with MeOH (30 mL×2). The combined filtrates were concentrated to dryness to provide product as a yellow solid (80 mg, 273 μmol, 88% yield).

LCMS: RT=1.055 min, MS cal.: 293.34, [M+H]⁺=294.2

¹H NMR (400 MHz, METHANOL-d₄) δ=8.88 (s, 1H), 7.84 (s, 1H), 7.11 (d, J=1.6, Hz, 1H), 7.09 (d, J=1.6, Hz, 1H), 4.65 (s, 2H), 3.90-3.87 (m, 3H), 3.82 (s, 3H)

### Preparation of Intermediate (5), Methyl 2-(chloromethyl)-4-methoxy-1-(thiazol-5-ylmethyl)-1H-benzo[d]imidazole-6-carboxylate (5

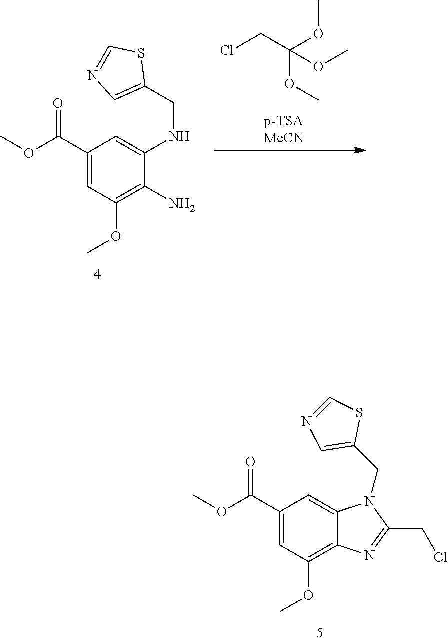

To a solution of methyl 4-amino-3-methoxy-5-((thiazol-5-ylmethyl)amino)benzoate, 4 (90 mg, 307 μmol, 1 eq), 2-chloro-1, 1, 1-trimethoxyethane (52.2 mg, 337 μmol, 45.5 μL, 1.1 eq) and p-TSA (2.64 mg, 15.3 μmol, 0.05 eq) in CH₃CN (1 mL) was degassed and purged with N₂ for 3 times, and then the mixture was stirred at 60° C. for 2 h under N₂ atmosphere. LC-MS showed starting material was consumed completely and desired mass was detected. The reaction mixture was concentrated to dryness to provide product as a white solid (100 mg, 284 μmol, 93% yield).

LCMS: RT=1.191 min, MS cal.: 351.81, [M+H]⁺=352.2

¹H NMR (400 MHz, METHANOL-d4) δ=8.98 (s, 1H), 8.00 (s, 1H), 7.91 (s, 1H), 7.49 (s, 1H), 5.96 (s, 2H), 5.05 (s, 2H), 4.06 (s, 3H), 3.94 (s, 3H).

### Preparation of Intermediate (6), Methyl 2-((4-(6-((4-cyano-2-fluorobenzyl)oxy)pyridin-2-yl)piperidin-1-yl)methyl)-4-methoxy-1-(thiazol-5-ylmethyl)-1H-benzo[d]imidazole-6-carboxylate

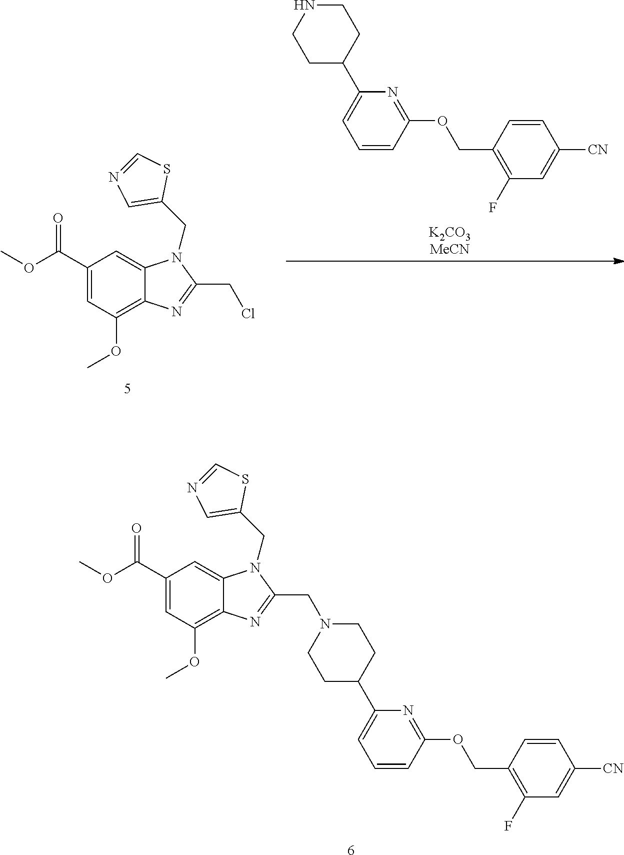

A mixture of methyl 2-(chloromethyl)-4-methoxy-1-(thiazol-5-ylmethyl)-1H-benzo[d]imidazole-6-carboxylate, 5 (100 mg, 284 μmol, 1 eq), 3-fluoro-4-(((6-(piperidin-4-yl)pyridin-2-yl)oxy)methyl)benzonitrile (88.5 mg, 284 μmol, 1 eq) and K₂CO₃ (118 mg, 853 μmol, 3 eq) in CH₃CN (1 mL) was degassed and purged with N₂ for 3 times, and then the mixture was stirred at 60° C. for 2 h under N₂ atmosphere. LC-MS showed starting material was consumed completely and desired mass was detected. The mixture was diluted with H₂O (30 mL), extracted with EtOAc (30 mL×3). The combined organic layers were dried over Na₂SO₄, filtered and the filtrate was concentrated to give a crude product. The residue was purified by column chromatography (SiO₂, Petroleum ether/Ethyl acetate=100/1 to 1/1) to provide product as a white solid (130 mg, 207 μmol, 73% yield).

LCMS: RT=1.470 min, MS cal.: 626.71, [M+H]⁺=627.3

¹H NMR (400 MHz, DMSO-d₆) δ=8.97 (s, 1H), 8.01 (s, 1H), 7.91-7.84 (m, 2H), 7.73-7.62 (m, 3H), 7.28 (s, 1H), 6.86 (d, J=7.2 Hz, 1H), 6.71 (d, J=8.0 Hz, 1H), 5.95 (s, 2H), 5.47 (s, 2H), 3.97 (s, 3H), 3.88-3.83 (m, 5H), 2.98-2.90 (m, 2H), 2.63-2.54 (m, 1H), 2.24-2.16 (m, 2H), 1.76-1.55 (m, 4H).

### Preparation of 2-((4-(6-((4-Cyano-2-fluorobenzyl)oxy)pyridin-2-yl)piperidin-1-yl)methyl)-4-methoxy-1-(thiazol-5-ylmethyl)-1H-benzo[d]imidazole-6-carboxylic acid, Compound 1 (7)

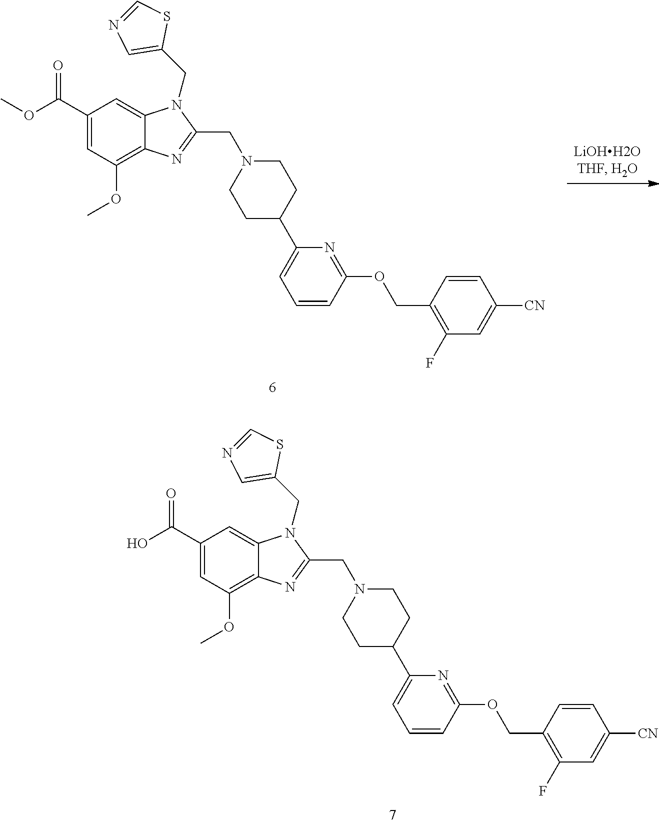 [SMILES: COC(=O)c1cc(OC)c2nc(CN3CCC(c4cccc(OCc5ccc(C#N)cc5F)n4)CC3)n(Cc3cncs3)c2c1 ; "LiOH·H2OTHF, H2O" ; "6" ; COc1cc(C(=O)O)cc2c1nc(CN1CCC(c3cccc(OCc4ccc(C#N)cc4F)n3)CC1)n2Cc1cncs1 ; "7"]

A mixture of methyl 2-((4-(6-((4-cyano-2-fluorobenzyl)oxy)pyridin-2-yl)piperidin-1-yl)methyl)-4-methoxy-1-(thiazol-5-ylmethyl)-1H-benzo[d]imidazole-6-carboxylate, 6 (130 mg, 207 μmol, 1 eq) and LiOH·H₂O (11.3 mg, 270 μmol, 1.3 eq) in THF (1 mL) and H₂O (0.3 mL) was degassed and purged with N₂ for 3 times, and then the mixture was stirred at 20° C. for 12 h under N₂ atmosphere. LC-MS showed starting material was consumed completely and desired mass was detected. The reaction mixture was dried under N₂. The crude product was pre-purified by prep-HPLC (NH₄HCO₃) to give product as a white solid (28.2 mg, 46.1 μmol, 22% yield).

LCMS: RT=2.564 min, MS cal.: 612.68, [M+H]⁺=613.2

HPLC: RT=10.088 min

¹H NMR (400 MHz, METHANOL-d4) δ=8.91 (s, 1H), 7.96 (s, 1H), 7.82 (s, 1H), 7.70-7.64 (m, 1H), 7.61-7.52 (m, 3H), 7.47 (s, 1H), 6.81 (d, J=7.2 Hz, 1H), 6.67 (d, J=8.0 Hz, 1H), 5.97 (s, 2H), 5.50 (s, 2H), 4.04 (s, 3H), 3.90 (s, 2H), 2.99 (m, 2H), 2.66-2.55 (m, 1H), 2.34-2.23 (m, 2H), 1.85-1.69 (m, 4H).

### Example A2. —Preparation of Compound 2. (S)-2-((4-(2-(5-Chloropyridin-2-yl)-2-methylbenzo[d][1,3]dioxol-4-yl)piperidin-1-yl)methyl)-4-(2-methoxyethoxy)-1-(thiazol-5-ylmethyl)-1H-benzo[d]imidazole-6-carboxylic acid (Compound 2) (7)

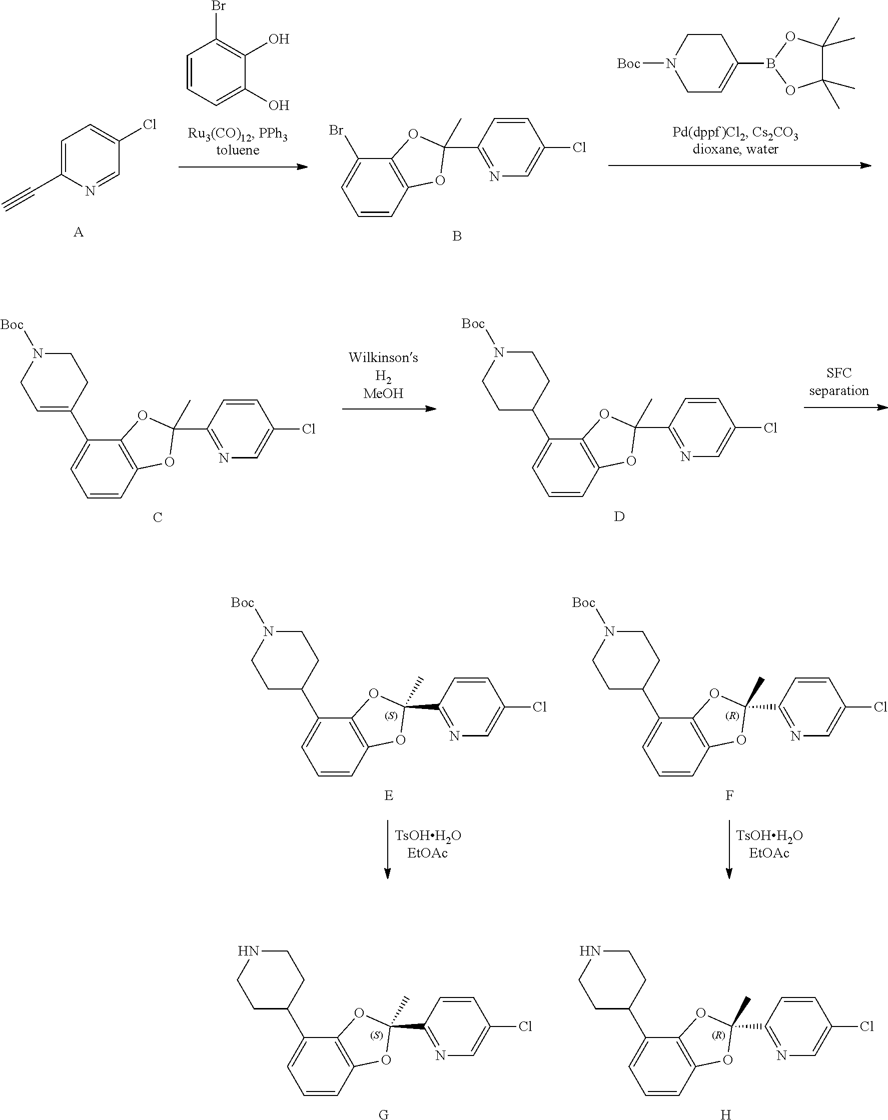

### Preparation of 2-(4-Bromo-2-methylbenzo[d][1,3]dioxol-2-yl)-5-chloropyridine, Intermediate B

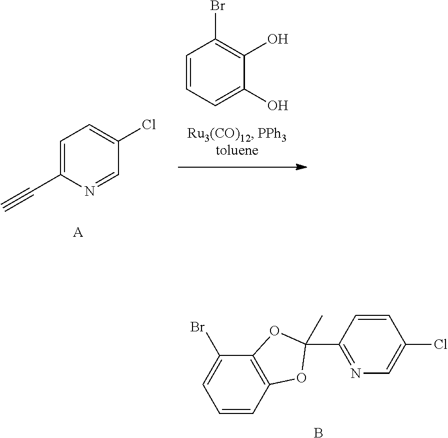

A mixture of 5-chloro-2-ethynylpyridine, A (15 g, 109 mmol, 1 eq), 3-bromobenzene-1,2-diol (41.2 g, 218 mmol, 2 eq), PPh₃ (2.86 g, 10.9 mmol, 0.1 eq) and Ru₃(CO)₁₂ (3.49 g, 5.45 mmol, 0.05 eq) in toluene (160 mL) was degassed and purged with N₂ for 3 times at 20° C., and then the mixture was stirred at 100° C. for 12 h under N₂ atmosphere. LCMS showed the starting material was consumed completely. The reaction mixture was poured into water (100 mL) and extracted with ethyl acetate (150 mL*2). The combined organic phase was concentrated in vacuo to give a residue. The residue was purified by column chromatography (SiO₂, Petroleum ether/Ethyl acetate=100/1 to 0/1) to provide product as a white oil (15 g, 45.9 mmol, 42% yield).

LCMS: RT=0.625 min, MS cal.: 326.6, [M+H]⁺=328.0

### Preparation of Intermediate (C), tert-Butyl 4-(2-(5-chloropyridin-2-yl)-2-methylbenzo[d][1,3]dioxol-4-yl)-3,6-dihydropyridine-1(2H)-carboxylate C

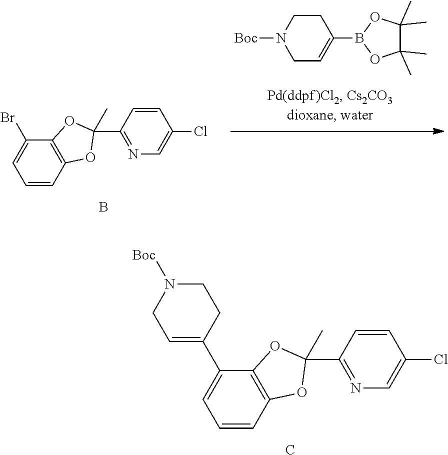

A mixture of 2-(4-bromo-2-methylbenzo[d][1,3]dioxol-2-yl)-5-chloropyridine, B (10 g, 30.6 mmol, 1 eq), tert-butyl 4-(4,4,5,5-tetramethyl-1,3,2-dioxaborolan-2-yl)-3,6-dihydropyridine-1(2H)-carboxylate (10.4 g, 33.7 mmol, 1.1 eq), Pd(dppf)Cl₂ (1.12 g, 1.53 mmol, 0.05 eq) and Cs₂CO₃ (11 g, 33.7 mmol, 1.1 eq) in dioxane (100 mL) and H₂O (20 mL) was degassed and purged with N₂ for 3 times at 20° C., and then the mixture was stirred at 90° C. for 3 h under N₂ atmosphere. LCMS showed the starting material was consumed completely. The reaction mixture was poured into water (100 mL) and extracted with ethyl acetate (150 mL*2). The combined organic phase was concentrated in vacuo to give a residue. The residue was purified by column chromatography (SiO₂, Petroleum ether/Ethyl acetate=100/1 to 0/1) to provide product as a yellow oil (10 g, 23.3 mmol, 76% yield).

¹H NMR (400 MHz, CHLOROFORM-d) δ=8.63-8.63 (d, J=2.4 Hz, 1H), 7.70-7.68 (dd, J=8.4 Hz, 1H), 7.58-7.56 (d, J=8.4 Hz, 1H), 6.82-6.76 (m, 2H), 6.75-6.74 (m, 1H), 6.39-6.33 (m, 1H), 4.11-4.10 (m, 2H), 3.64-3.62 (m, 2H), 2.56 (br s, 2H), 2.08 (s, 3H), 1.49 (s, 9H)

### Preparation of Intermediate D, tert-Butyl 4-(2-(5-chloropyridin-2-yl)-2-methylbenzo[d][1,3]dioxol-4-yl)piperidine-1-carboxylate

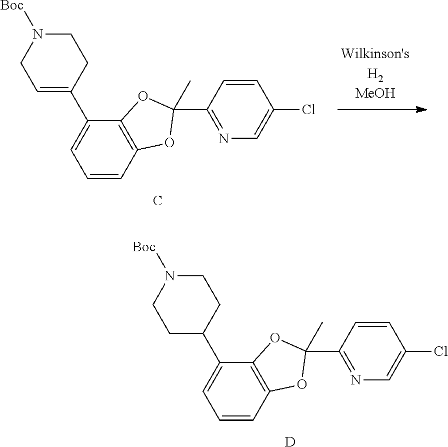

To a solution of tert-butyl 4-(2-(5-chloropyridin-2-yl)-2-methylbenzo[d][1,3]dioxol-4-yl)-3,6-dihydropyridine-1(2H)-carboxylate, C, (10 g, 23.3 mmol, 1 eq) in MeOH (100 mL) was added Wilkinson's catalyst (2.16 g, 2.33 mmol, 0.1 eq) under N₂. The suspension was degassed under vacuum and purged with H₂ several times. The mixture was stirred under H₂ (50 psi) at 60° C. for 24 h. LCMS showed the starting material was consumed completely. The reaction mixture was filtered and the filter was concentrated. The residue was purified by column chromatography (SiO₂, Petroleum ether/Ethyl acetate=100/1 to 0/1) to provide product as a yellow oil (8 g, 18.6 mmol, 80% yield).

LCMS: RT=2.450 min, MS cal.: 430.93 [M−55]⁺=431.1

¹H NMR (400 MHz, CHLOROFORM-d) δ ppm 8.63-8.62 (d, J=2.4 Hz, 1H) 7.71-7.68 (dd, J=8.4, 2.4 Hz, 1H) 7.59-7.56 (d, J=8.4 Hz, 1H) 6.80-6.78 (m, 1H) 6.73-6.69 (m, 2H) 4.23-4.22 (m, 2H) 2.88-2.81 (m, 3H) 2.05 (s, 3H) 1.88-1.64 (m, 4H) 1.48-1.24 (m, 9H).

### Preparation of tert-butyl(S)-4-(2-(5-chloropyridin-2-yl)-2-methylbenzo[d][1,3]dioxol-4-yl)piperidine-1-carboxylate Intermediate (E) and tert-butyl (R)-4-(2-(5-chloropyridin-2-yl)-2-methylbenzo[d][1,3|dioxol-4-yl)piperidine-1-carboxylate, Intermediate F

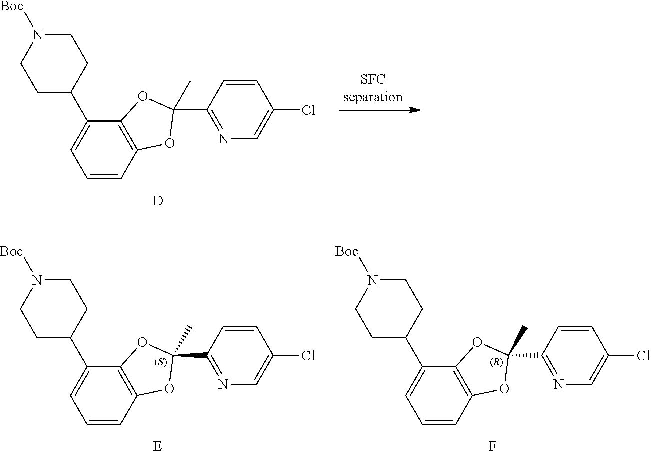

tert-Butyl 4-(2-(5-chloropyridin-2-yl)-2-methylbenzo[d][1,3]dioxol-4-yl)piperidine-1-carboxylate, (D), (2 g, 4.64 mmol, 1 eq) was purified by SFC (column: DAICEL CHIRALPAK AD (250 mm*30 mm, 10 um); mobile phase: [CO₂-IPA (0.1% NH3H2O)]; B %: 20%, isocratic elution mode) to provide tert-butyl(S)-4-(2-(5-chloropyridin-2-yl)-2-methylbenzo[d][1,3]dioxol-4-yl)piperidine-1-carboxylate, (E), as a white solid (1.5 g, 3.48 mmol, 75% yield) and tert-butyl (R)-4-(2-(5-chloropyridin-2-yl)-2-methylbenzo[d][1,3]dioxol-4-yl)piperidine-1-carboxylate, (F), as a white solid (1.5 g, 3.48 mmol, 75% yield).

### Preparation of (S)-5-Chloro-2-(2-methyl-4-(piperidin-4-yl)benzo[d][1,3]dioxol-2-yl)pyridine, Intermediate G, and (R)-5-chloro-2-(2-methyl-4-(piperidin-4-yl)benzo[d][1,3]dioxol-2-yl)pyridine, Intermediate H

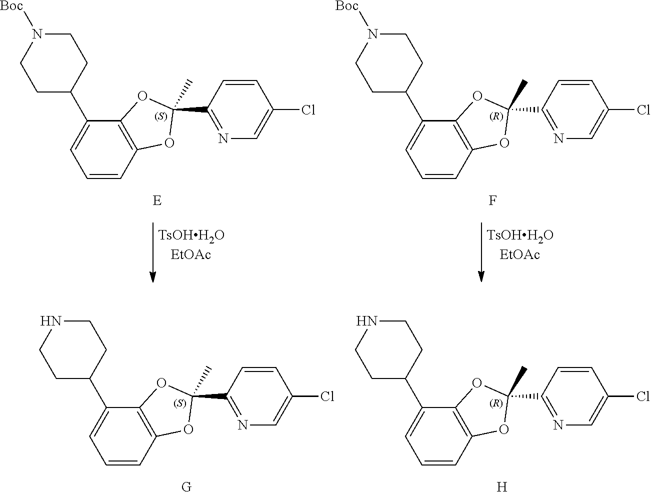

A mixture of tert-butyl(S)-4-(2-(5-chloropyridin-2-yl)-2-methylbenzo[d][1,3] dioxol-4-yl)piperidine-1-carboxylate, (E), (700 mg, 1.62 mmol, 1 eq) and TsOH·H₂O (699 mg, 4.06 mmol, 2.5 eq) in EtOAc (14 mL) was degassed and purged with N₂ for 3 times, and then the mixture was stirred at 60° C. for 2 h under N₂ atmosphere. LCMS showed the starting material was consumed completely. The aqueous layer was neutralized by aq. NaHCO₃ at −5° C.˜5° C. to pH around 7˜8. The mixture was poured into water (50 mL) and extracted with ethyl acetate (50 mL*2). The combined organic phase was concentrated in vacuo to provide (S)-5-Chloro-2-(2-methyl-4-(piperidin-4-yl)benzo[d][1,3]dioxol-2-yl)pyridine, (G), as a white solid (744 mg, crude).

LCMS: RT=0.696 min, MS cal.: 330.1, [M+H]⁺=331.1

A mixture of tert-butyl (R)-4-(2-(5-chloropyridin-2-yl)-2-methylbenzo[d][1,3]dioxol-4-yl)piperidine-1-carboxylate (F) (0.7 g, 1.62 mmol, 1 eq) and TosOH·H₂O (699 mg, 4.06 mmol, 2.5 eq) in EtOAc (14 mL) was degassed and purged with N₂ for 3 times, and then the mixture was stirred at 60° C. for 2 h under N₂ atmosphere. LCMS showed the starting material was consumed completely. The aqueous layer was neutralized by aq. NaHCO₃ at −5° C.˜5° C. to pH around 7˜8. The reaction mixture was poured into water (50 mL) and extracted with ethyl acetate (50 mL*2). The combined organic phase was concentrated in vacuo to give (R)-5-chloro-2-(2-methyl-4-(piperidin-4-yl)benzo[d][1,3]dioxol-2-yl)pyridine (H) as a white solid (530 mg, 1.60 mmol, 99% yield).

LCMS: RT=0.416 min, MS cal.: 330.1, [M+H]⁺=331.2

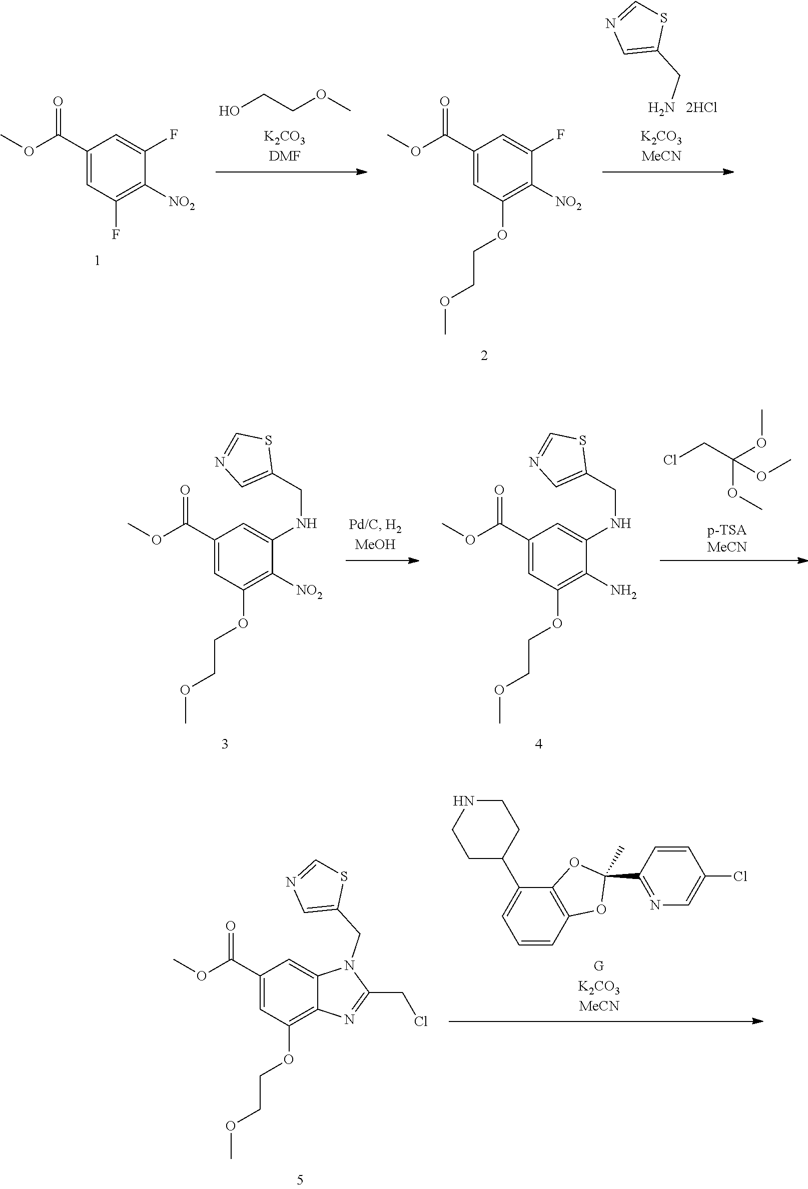

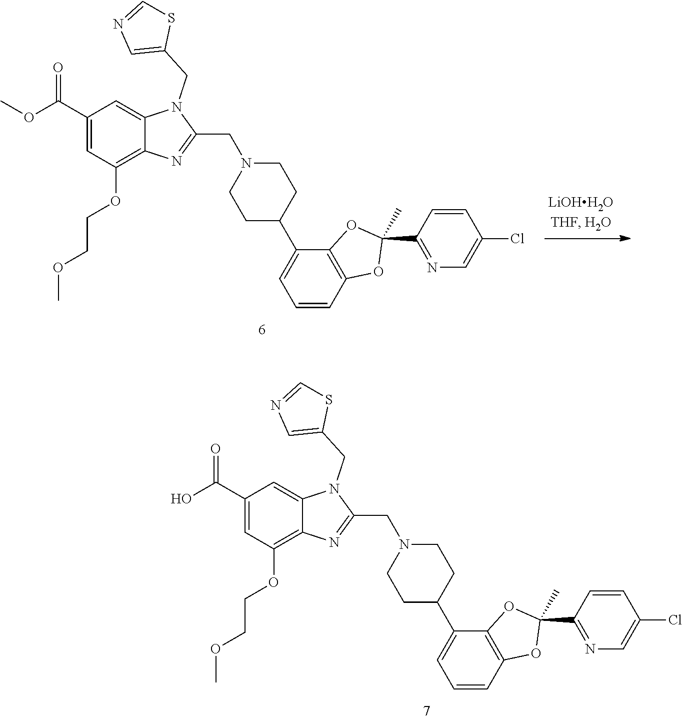 [SMILES: COCCOc1cc(C(=O)OC)cc2c1nc(CN1CCC(c3cccc4c3O[C@@](C)(c3ccc(Cl)cn3)O4)CC1)n2Cc1cncs1 ; COCCOc1cc(C(=O)O)cc2c1nc(CN1CCC(c3cccc4c3O[C@@](C)(c3ccc(Cl)cn3)O4)CC1)n2Cc1cncs1]

### Preparation of Intermediate (2), Methyl 3-fluoro-5-(2-methoxyethoxy)-4-nitrobenzoate

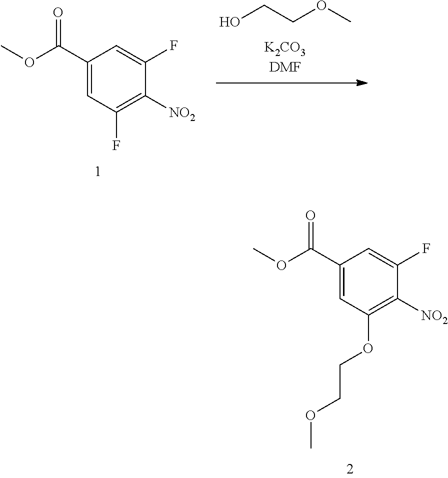

To a solution of 2-methoxyethan-1-ol 1 (1.8 g, 23.7 mmol, 1 eq) in DMF (30 mL) was added methyl 3,5-difluoro-4-nitrobenzoate (5.14 g, 23.7 mmol, 1 eq) and K₂CO₃ (9.81 g, 71 mmol, 3 eq) and the mixture was stirred at 60° C. for 12 h under N₂ atmosphere. LC-MS showed starting material was consumed completely and one main peak with desired mass was detected. The reaction mixture was diluted with H₂O (20 mL) and extracted with EtOAc (20 mL*3). The combined organic phase was dried over Na₂SO₄, filtered and concentrated under reduced pressure to give a residue. The residue was purified by column chromatography (SiO₂, Petroleum ether/Ethyl acetate=100/1 to 10/1) to provide product as a yellow oil (2 g, 7.32 mmol, 31% yield).

LCMS: RT=1.622 min, MS cal.: 273.0, [M+H]⁺=274.3

### Preparation of Intermediate (3)

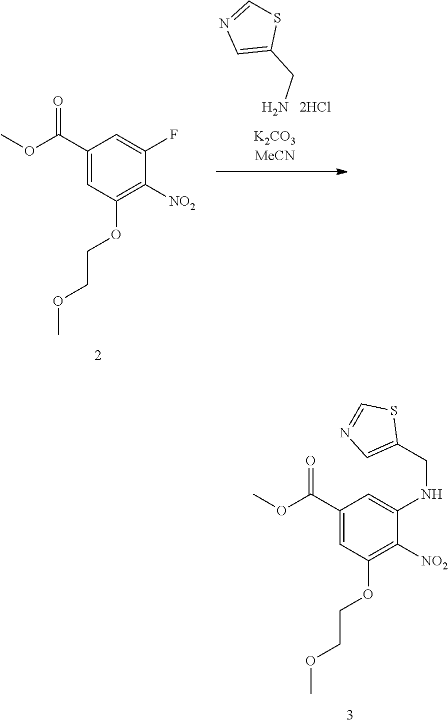 [SMILES: COCCOc1cc(C(=O)OC)cc(F)c1[N+](=O)[O-] ; NCc1cncs1 ; COCCOc1cc(C(=O)OC)cc(NCc2cncs2)c1[N+](=O)[O-]]

A mixture of methyl 3-fluoro-5-(2-methoxyethoxy)-4-nitrobenzoate 2 (400 mg, 1.46 mmol, 1 eq), thiazol-5-ylmethanamine (1.10 g, 7.32 mmol, 5 eq, HCl), K₂CO₃ (809 mg, 5.86 mmol, 4 eq) and NaI (439 mg, 2.93 mmol, 2 eq) in CH₃CN (10 mL) was degassed and purged with N₂ for 3 times, and then the mixture was stirred at 60° C. for 12 hr under N₂ atmosphere. LC-MS showed ˜48% of starting material remained and ˜45% of desired compound was detected. The reaction mixture was diluted with H₂O (5 mL) and extracted with EtOAc 30 mL (10 mL*3). The combined organic phase was dried over Na₂SO₄, filtered, and concentrated under reduced pressure to give a residue. The residue was purified by column chromatography (SiO₂, DCM/MeOH=100/1 to 10/1) to provide product as a yellow oil (150 mg, 408 μmol, 28% yield).

LCMS: RT=1.393 min, MS cal.: 367.0, [M+H]⁺=368.2

¹H NMR (400 MHz, CHLOROFORM-d) δ=8.77 (s, 1H), 7.86 (s, 1H), 7.16 (d, J=1.2 Hz, 1H), 7.05 (d, J=1.2 Hz, 1H), 6.08 (br t, J=5.5 Hz, 1H), 4.71 (d, J=5.5 Hz, 2H), 4.33-4.16 (m, 2H), 3.92 (s, 3H), 3.83-3.73 (m, 2H), 3.44 (s, 3H).

### Preparation of Intermediate (4)

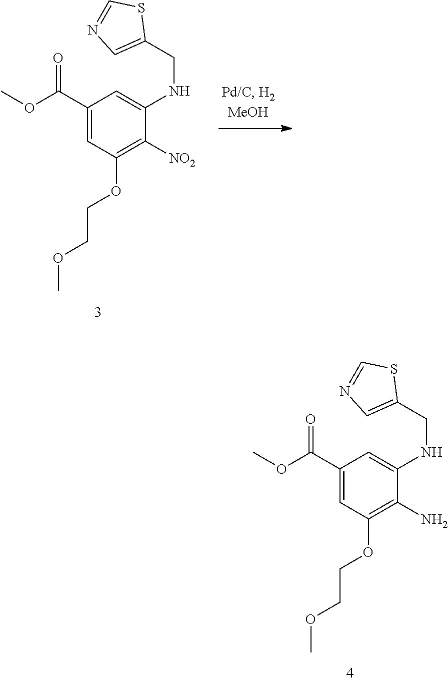 [SMILES: COCCOc1cc(C(=O)OC)cc(NCc2cncs2)c1[N+](=O)[O-] ; COCCOc1cc(C(=O)OC)cc(NCc2cncs2)c1N]

To a solution of methyl 3-(2-methoxyethoxy)-4-nitro-5-((thiazol-5-ylmethyl)amino)benzoate 3 (150 mg, 408 μmol, 1 eq) in MeOH (1 mL) was added Pd/C (1.30 g, 1.22 mmol, 10%, 3 eq) under N₂ atmosphere. The suspension was degassed and purged with H₂ for 3 times. The mixture was stirred under H₂ (15 psi) at 25° C. for 1 h. LC-MS showed starting material was consumed completely and one main peak with desired mass was detected. The resulting product was diluted in MeOH (10 mL) and filtered. The filtrate was concentrated in vacuo to provide product as a colorless oil (130 mg, 385 μmol, 94% yield).

LCMS: RT=1.083 min, MS cal.: 337.1, [M+H]⁺=338.2

### Preparation of Intermediate (5) Methyl 2-(chloromethyl)-4-(2-methoxyethoxy)-1-(thiazol-5-ylmethyl)-1H-benzo[d]imidazole-6-carboxylate

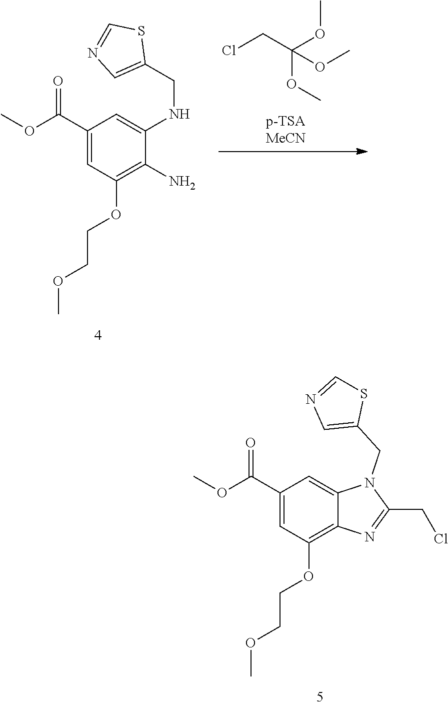

A mixture of methyl 4-amino-3-(2-methoxyethoxy)-5-((thiazol-5-ylmethyl)amino)benzoate 4 (110 mg, 326 μmol, 1 eq), 2-chloro-1,1,1-trimethoxyethane (60.48 mg, 391 μmol, 52.7 μL, 1.2 eq) and TosOH (5.61 mg, 32.6 μmol, 0.1 eq) in CH₃CN (1 mL) was degassed and purged with N₂ for 3 times and then the mixture was stirred at 60° C. for 1 h under N₂ atmosphere. LC-MS showed starting material was consumed completely and one main peak with desired mass was detected. The reaction mixture was concentrated under reduced pressure to remove solvent. Product was obtained as a colorless oil (120 mg, 303 μmol, 93% yield).

LCMS: RT=1.221 min, MS cal.: 395.0, [M+H]⁺=396.2

1H NMR (400 MHz, CHLOROFORM-d) δ=8.78 (s, 1H), 7.87 (s, 1H), 7.79-7.74 (s, 1H), 7.48 (s, 1H), 5.76 (s, 2H), 4.90-4.85 (s, 2H), 4.44 (m, 2H), 3.95 (s, 3H), 3.92-3.85 (m, 2H), 3.49-3.44 (m, 3H)

### Preparation of Intermediate (6) Methyl (S)-2-((4-(2-(5-chloropyridin-2-yl)-2-methylbenzo[d][1,3]dioxol-4-yl)piperidin-1-yl)methyl)-4-(2-methoxyethoxy)-1-(thiazol-5-ylmethyl)-1H-benzo[d]imidazole-6-carboxylate

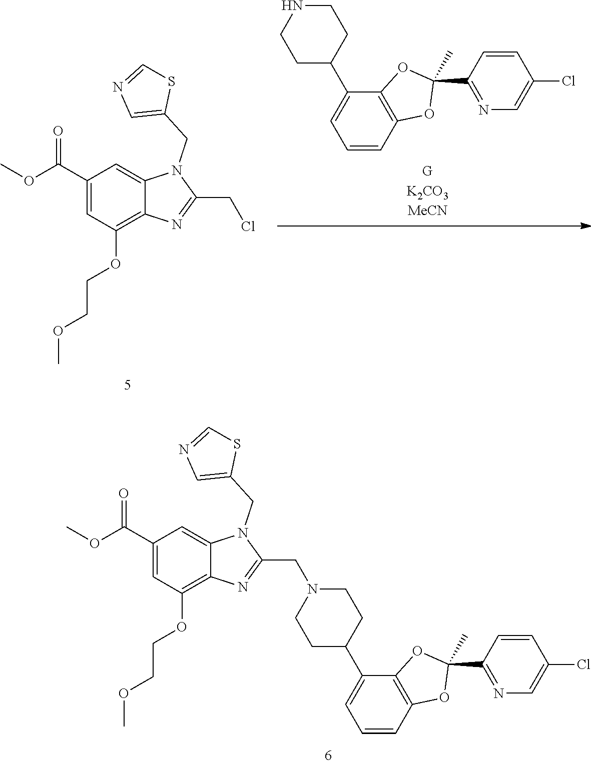 [SMILES: COCCOc1cc(C(=O)OC)cc2c1nc(CCl)n2Cc1cncs1 ; C[C@]1(c2ccc(Cl)cn2)Oc2cccc(C3CCNCC3)c2O1 ; "GK2CO3MeCN" ; COCCOc1cc(C(=O)OC)cc2c1nc(CN1CCC(c3cccc4c3O[C@@](C)(c3ccc(Cl)cn3)O4)CC1)n2Cc1cncs1]

A mixture of methyl 2-(chloromethyl)-4-(2-methoxyethoxy)-1-(thiazol-5-ylmethyl)-1H-benzo[d]imidazole-6-carboxylate 5 (46 mg, 116.20 μmol, 1 eq), (S)-5-chloro-2-(2-methyl-4-(piperidin-4-yl)benzo[d][1,3]dioxol-2-yl)pyridine (57.7 mg, 174 μmol, 1.5 eq) and K₂CO₃ (48.2 mg, 349 μmol, 3 eq) in CH₃CN (1 mL) was degassed and purged with N₂ for 3 times, and then the mixture was stirred at 60° C. for 2 h under N₂ atmosphere. LC-MS showed starting material was consumed completely and one main peak with desired mass was detected. The mixture was diluted with H₂O (10 mL), extracted with EtOAc (10 mL*3). The combined organic layer was dried over Na₂SO₄, filtered and the filtrate was concentrated to give a crude product. The residue was purified by prep-HPLC (column: Waters Xbridge Prep OBD C18 150*40 mm*10 um; mobile phase: [H₂O (10 mM NH₄HCO₃)-ACN]; gradient: 40%-80% B over 8.0 min) to provide product as a white solid (50 mg, 72.4 μmol, 62% yield).

LCMS: RT=0.725 min, MS cal.: 689.2, [M+H]⁺=690.3

### Preparation of Compound 2, (S)-2-((4-(2-(5-chloropyridin-2-yl)-2-methylbenzo[d][1,3]dioxol-4-yl)piperidin-1-yl)methyl)-4-(2-methoxyethoxy)-1-(thiazol-5-ylmethyl)-1H-benzo[d]imidazole-6-carboxylic acid (7)

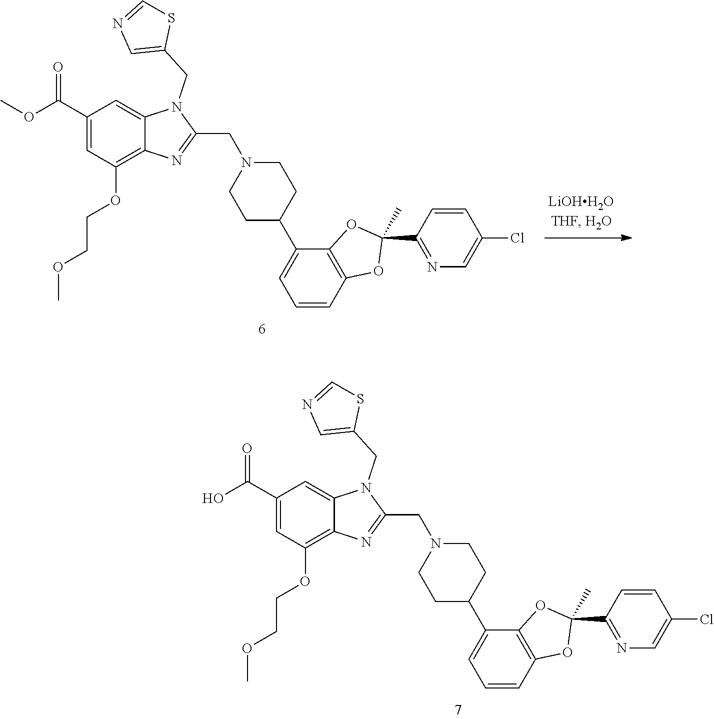 [SMILES: COCCOc1cc(C(=O)OC)cc2c1nc(CN1CCC(c3cccc4c3O[C@@](C)(c3ccc(Cl)cn3)O4)CC1)n2Cc1cncs1 ; COCCOc1cc(C(=O)O)cc2c1nc(CN1CCC(c3cccc4c3O[C@@](C)(c3ccc(Cl)cn3)O4)CC1)n2Cc1cncs1]

To a solution of methyl (S)-2-((4-(2-(5-chloropyridin-2-yl)-2-methylbenzo[d][1,3]dioxol-4-yl)piperidin-1-yl)methyl)-4-(2-methoxyethoxy)-1-(thiazol-5-ylmethyl)-1H-benzo[d]imidazole-6-carboxylate 6 (40 mg, 57.9 μmol, 1 eq) in THF (0.7 mL) was added LiOH·H₂O (3.65 mg, 86.9 μmol, 1.5 eq) in H₂O (0.3 mL). The mixture was stirred at 25° C. for 12 h. LC-MS showed starting material was consumed completely and one main peak with desired mass was detected. The reaction mixture was concentrated under reduced pressure to remove solvent. The residue was purified by prep-HPLC (column: Waters Xbridge BEH C18 100*30 mm*10 um; mobile phase: [H2O (10 mM NH₄HCO₃)-ACN]; gradient: 25%-55% B over 8.0 min) to provide product as a white solid (32.7 mg, 48.4 μmol, 83% yield).

LCMS: RT=1.615 min, MS cal.: 675.2, [M+H]⁺=676.2

HPLC: RT=10.576 min

¹H NMR (400 MHz, DMSO-d₆) δ=8.96 (s, 1H), 8.72 (s, 1H), 8.02-7.98 (m, 2H), 7.79 (s, 1H), 7.59 (d, J=8.4 Hz, 1H), 7.27 (s, 1H), 6.82-6.75 (m, 2H), 6.72-6.68 (m, 1H), 5.90 (s, 2H), 4.34-4.30 (m, 2H), 3.84 (s, 2H), 3.76-3.73 (m, 2H), 3.34 (s, 3H), 3.01-2.89 (m, 2H), 2.69-2.63 (m, 1H), 2.24-2.13 (m, 2H), 2.00 (s, 3H), 1.74-1.63 (m, 4H).

### Example A3. —Preparation of Compound 3. (R)-2-((4-(2-(5-Chloropyridin-2-yl)-2-methylbenzo[d][1,3]dioxol-4-yl)piperidin-1-yl)methyl)-4-(2-methoxyethoxy)-1-(thiazol-5-ylmethyl)-1H-benzo[d]imidazole-6-carboxylic acid (Compound 3) (3)

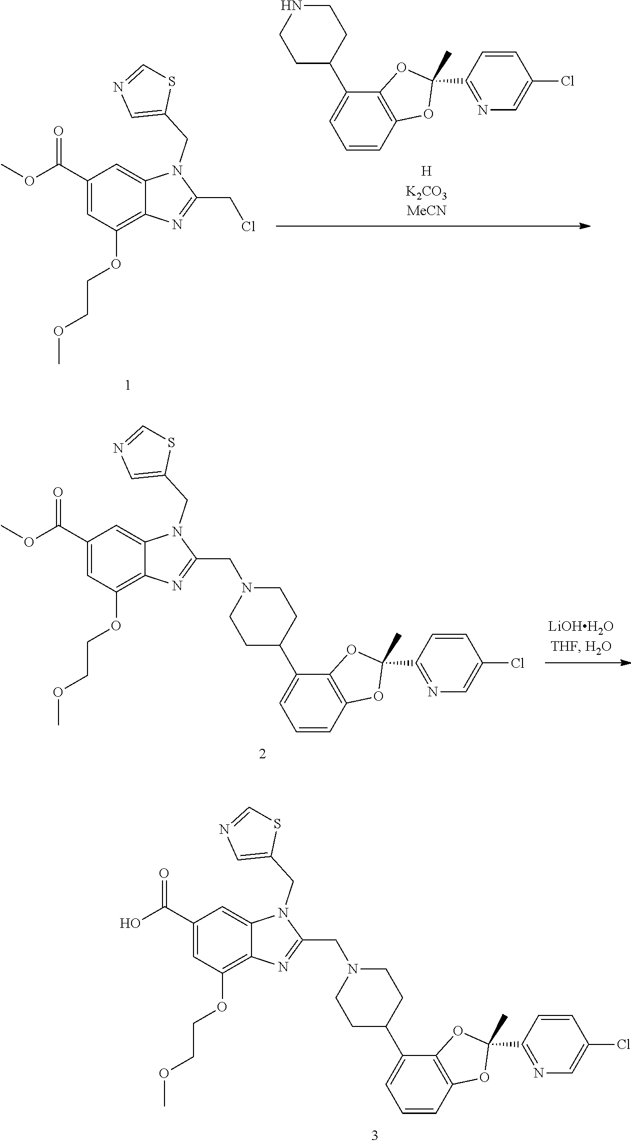

### Preparation of Methyl (R)-2-((4-(2-(5-chloropyridin-2-yl)-2-methylbenzo[d][1,3]dioxol-4-yl)piperidin-1-yl)methyl)-4-(2-methoxyethoxy)-1-(thiazol-5-ylmethyl)-1H-benzo[d]imidazole-6-carboxylate, Intermediate (2)

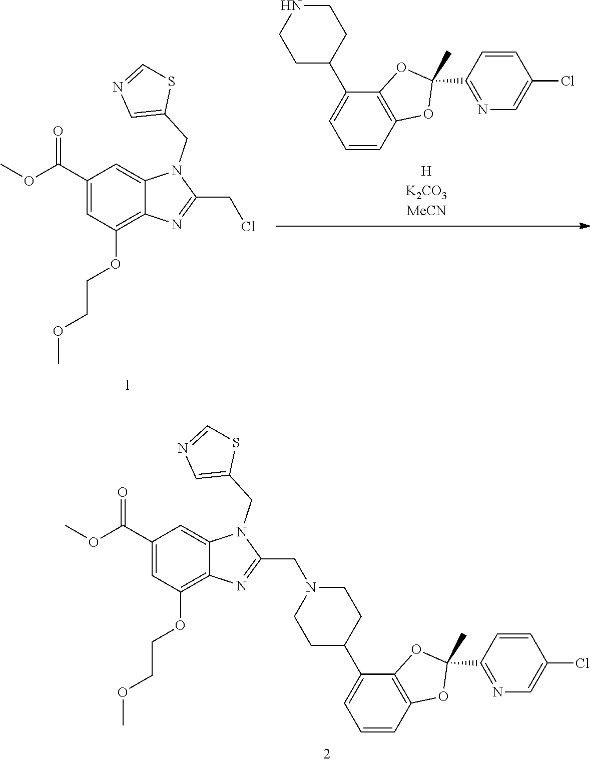

A mixture of methyl 2-(chloromethyl)-4-(2-methoxyethoxy)-1-(thiazol-5-ylmethyl)-1H-benzo[d]imidazole-6-carboxylate 1 (95.7 mg, 242 μmol, 1 eq), (R)-5-chloro-2-(2-methyl-4-(piperidin-4-yl)benzo[d][1,3]dioxol-2-yl)pyridine H (80 mg, 242 μmol, 1 eq) and K₂CO₃ (100 mg, 726 μmol, 3 eq) in CH₃CN (1 mL) was degassed and purged with N₂ for 3 times, and then the mixture was stirred at 60° C. for 2 h under N₂ atmosphere. LC-MS showed starting material was consumed completely and one main peak with desired mass was detected. The residue was diluted with H₂O (30 mL) and extracted with EtOAc (30 mL*3). The combined organic layers were washed with H₂O (30 mL), dried over Na₂SO₄, filtered, and concentrated under reduced pressure to give a residue. The residue was purified by column chromatography (SiO₂, Petroleum ether/Ethyl acetate=100/1 to 1/1) to provide product as a white solid (70 mg, 101 μmol, 42% yield).

LCMS: RT=1.525 min, MS cal.: 689.2, [M+H]⁺=690.3

### Preparation of Compound 3, (R)-2-((4-(2-(5-Chloropyridin-2-yl)-2-methylbenzo[d][1,3]dioxol-4-yl)piperidin-1-yl)methyl)-4-(2-methoxyethoxy)-1-(thiazol-5-ylmethyl)-1H-benzo[d]imidazole-6-carboxylic acid (3)

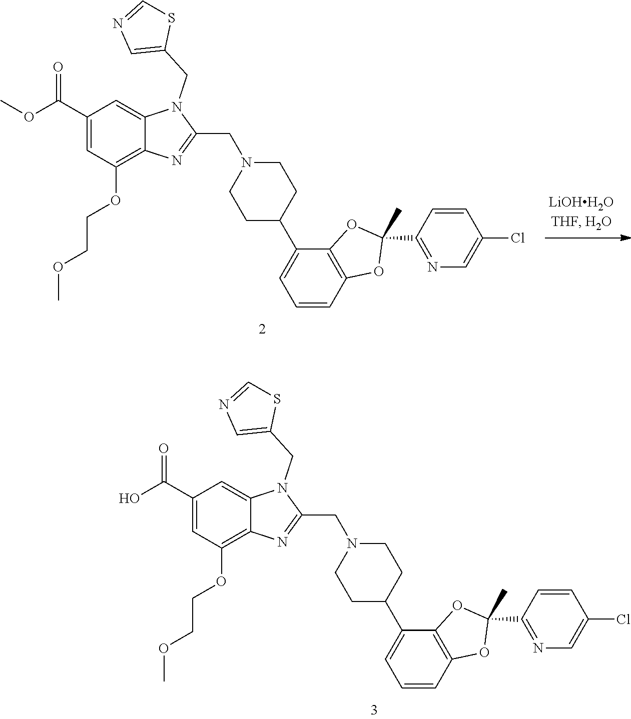 [SMILES: COCCOc1cc(C(=O)OC)cc2c1nc(CN1CCC(c3cccc4c3O[C@](C)(c3ccc(Cl)cn3)O4)CC1)n2Cc1cncs1 ; COCCOc1cc(C(=O)O)cc2c1nc(CN1CCC(c3cccc4c3O[C@](C)(c3ccc(Cl)cn3)O4)CC1)n2Cc1cncs1]

A mixture of methyl (R)-2-((4-(2-(5-chloropyridin-2-yl)-2-methylbenzo[d][1,3]dioxol-4-yl)piperidin-1-yl)methyl)-4-(2-methoxyethoxy)-1-(thiazol-5-ylmethyl)-1H-benzo[d]imidazole-6-carboxylate 2 (60 mg, 86.9 μmol, 1 eq) in THF (0.7 mL) and LiOH·H₂O (5.47 mg, 130 μmol, 1.5 eq) in H₂O (0.3 mL) was degassed and purged with N₂ for 3 times, and then the mixture was stirred at 25° C. for 12 h under N₂ atmosphere. LC-MS showed starting material was consumed completely and one main peak with desired mass was detected. The residue was purified by prep-HPLC (neutral condition column: Waters Xbridge Prep OBD C18 150*40 mm*10 um; mobile phase: [H₂O (10 Mm NH₄HCO₃)-ACN]; gradient: 25%-65% B over 8.0 min) to provide product as a white solid (21.0 mg, 31.1 μmol, 36% yield).

LCMS: ET43536-1161-P1A1, RT=1.606 min, MS cal.: 675.1, [M+H]⁺=676.3

1H NMR (400 MHz, DMSO-d6) δ=8.96 (s, 1H), 8.72 (s, 1H), 8.03-7.95 (m, 2H), 7.79 (s, 1H), 7.59 (d, J=8.8 Hz, 1H), 7.26 (s, 1H), 6.84-6.75 (m, 2H), 6.70 (m, 1H), 5.90 (s, 2H), 4.31 (dd, J=3.7, 5.2 Hz, 2H), 3.84 (s, 2H), 3.74 (dd, J=3.7, 5.1 Hz, 2H), 3.34 (s, 3H), 2.95 (br d, J=9.9 Hz, 2H), 2.66-2.58 (m, 1H), 2.26-2.12 (m, 2H), 2.00 (s, 3H), 1.76-1.60 (m, 4H).

### Example A4. —Preparation of Compound 4. (S)-4-((2,5,8,11,14-pentaoxahexadecan-16-yl)oxy)-2-((4-(2-(5-chloropyridin-2-yl)-2-methylbenzo[d][1,3]dioxol-4-yl)piperidin-1-yl)methyl)-1-(thiazol-5-ylmethyl)-1H-benzo[d]imidazole-6-carboxylic acid (Compound 4) (7)

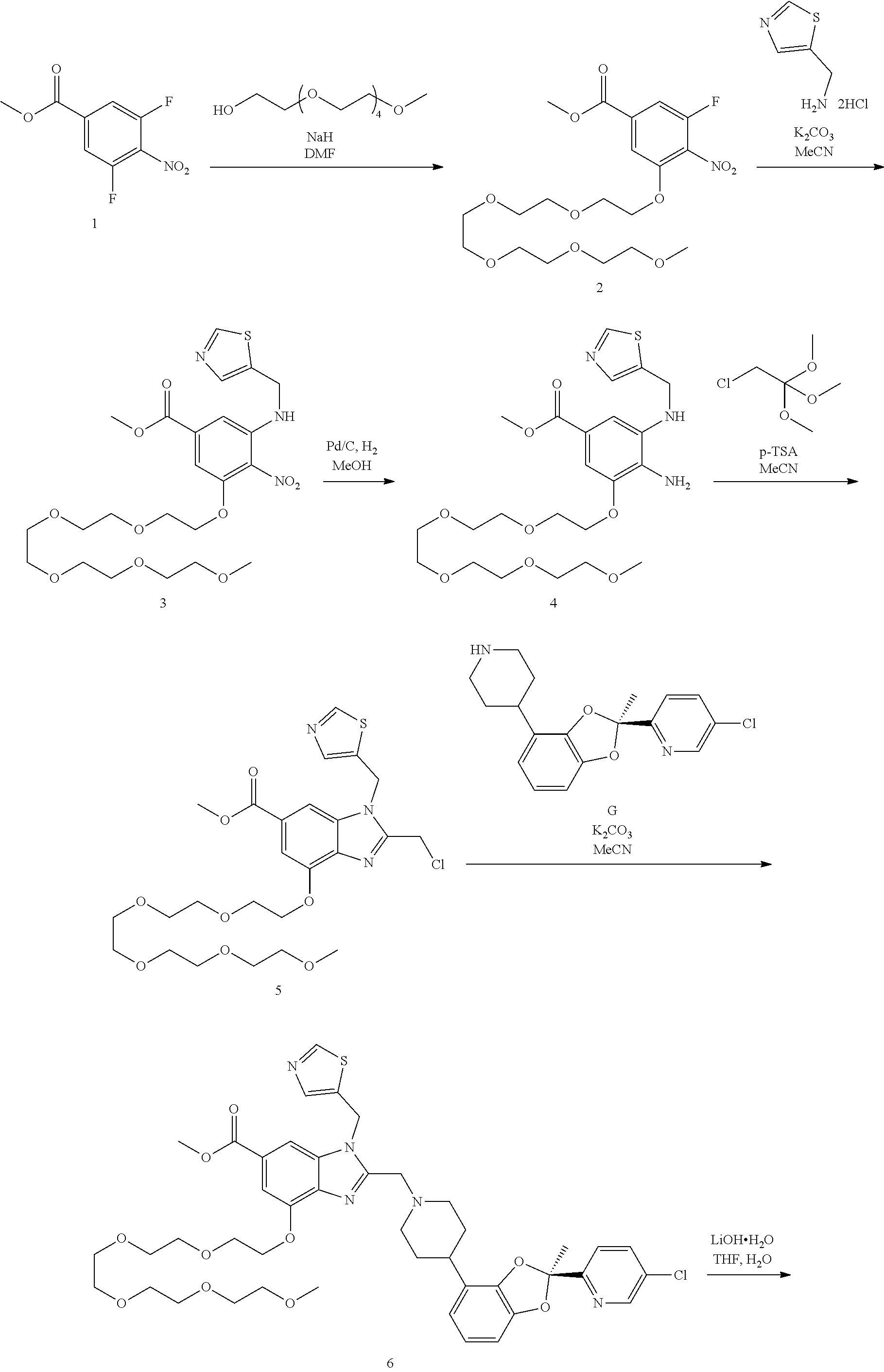

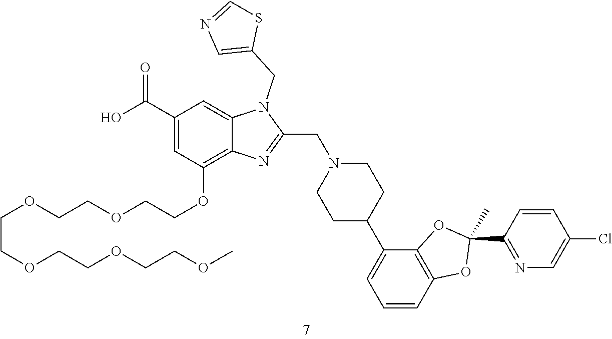 [SMILES: COCCOCCOCCOCCOCCOc1cc(C(=O)O)cc2c1nc(CN1CCC(c3cccc4c3O[C@@](C)(c3ccc(Cl)cn3)O4)CC1)n2Cc1cncs1]

### Preparation of Intermediate (2), Methyl 3-((2,5,8,11,14-pentaoxahexadecan-16-yl)oxy)-5-fluoro-4-nitrobenzoate (2)

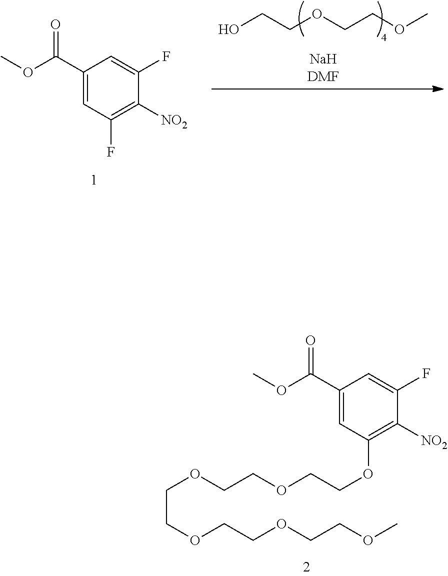

To a solution of 2,5,8, 11, 14-pentaoxahexadecan-16-ol (1.2 g, 4.76 mmol, 1 eq) in DMF (10 mL) was added NaH (190 mg, 4.76 mmol, 60% purity, 1 eq). The mixture was stirred at 0° C. for 0.5 hr. Then the mixture was added with methyl 3,5-difluoro-4-nitrobenzoate 1 (1.03 g, 4.76 mmol, 1 eq) at 0° C. The mixture was stirred at 20° C. for 2 h. The reaction mixture was quenched by addition NH₄Cl 10 mL at 0° C., and then extracted with EtOAc 30 mL (10 mL*3). The combined organic layers were dried over Na₂SO₄, filtered, and concentrated under reduced pressure to give a residue. The residue was purified by column chromatography (SiO₂, Dichloromethane: Methanol=100/1 to 10/1) (Rf=0.3) to provide product as yellow oil (1 g, crude).

LCMS: Rt=1.739 min, MS cal.: 449.17, [M+H₂O]⁺=467.2

### Preparation Intermediate (3) Methyl 3-((2,5,8,11,14-pentaoxahexadecan-16-yl)oxy)-4-nitro-5-((thiazol-5-ylmethyl)amino)benzoate

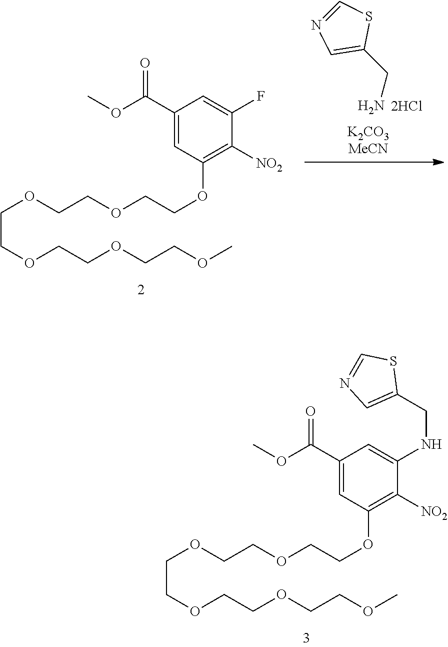

To a solution of thiazol-5-ylmethanamine (1.01 g, 6.68 mmol, 5 eq, HCl) in CH₃CN (6 mL) was added K₂CO₃ (554 mg, 4.01 mmol, 3 eq), NaI (400 mg, 2.67 mmol, 2 eq) and methyl 3-((2,5,8,11,14-pentaoxahexadecan-16-yl)oxy)-5-fluoro-4-nitrobenzoate 2 (600 mg, 1.34 mmol, 1 eq). The mixture was stirred at 60° C. for 72 h. LC-MS showed starting material was consumed completely and desired mass was detected. The residue was diluted with H₂O 20 mL and extracted with EtOAc 30 mL (30 mL*3). The combined organic layers were dried over Na₂SO₄, filtered, and concentrated under reduced pressure to give a residue. The residue was purified by column chromatography (SiO₂, Dichloromethane: Methanol=100/1 to 10/1) (Rf=0.53) to provide product as a yellow oil (400 mg, 736 μmol, 55% yield).

LCMS: Rt=1.368 min, MS cal.: 543.59, [M+H]⁺=544.3

### Preparation of Intermediate (4)

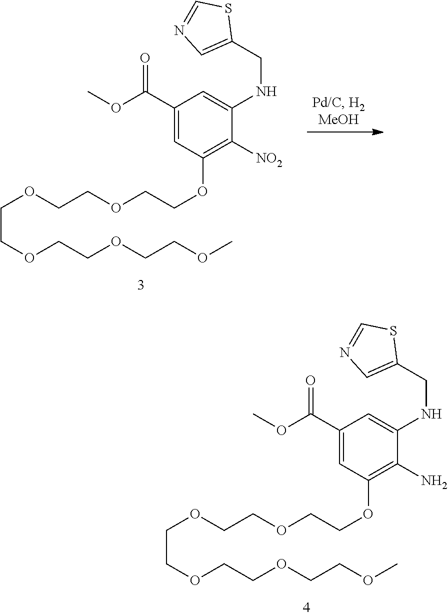 [SMILES: COCCOCCOCCOCCOCCOc1cc(C(=O)OC)cc(NCc2cncs2)c1[N+](=O)[O-] ; "Pd/C, H2MeOH" ; COCCOCCOCCOCCOCCOc1cc(C(=O)OC)cc(NCc2cncs2)c1N]

To a solution of methyl 3-((2,5,8,11,14-pentaoxahexadecan-16-yl)oxy)-4-nitro-5-((thiazol-5-ylmethyl)amino)benzoate 3 (80 mg, 147.17 μmol, 1 eq) in MeOH (1 mL) was added Pd/C (80.00 mg, 75.17 μmol, 10% purity) under N₂ atmosphere. The suspension was degassed and purged with H₂ for 3 times. The mixture was stirred under H₂ (15 Psi) at 20° C. for 2h. LC-MS showed starting material was consumed completely and desired mass was detected. The mixture was filtered through a Celite pad, and the filtrate was concentrated to provide crude product as a white solid (60 mg, 117 μmol, 79% yield).

LCMS: Rt=1.155 min, MS cal.: 513.61, [M+H]⁺=514.3

¹H NMR (400 MHz, CHLOROFORM-d) δ=8.75 (s, 1H), 7.86 (s, 1H), 7.23 (d, J=1.6 Hz, 1H), 7.19 (d, J=1.6 Hz, 1H), 4.61 (s, 2H), 4.22 (m, 2H), 3.87-3.83 (m, 5H), 3.74-3.71 (m, 2H), 3.68-3.63 (m, 10H), 3.63-3.60 (m, 2H), 3.52-3.49 (m, 2H), 3.33 (s, 3H).

### Preparation of Intermediate (5) (5)

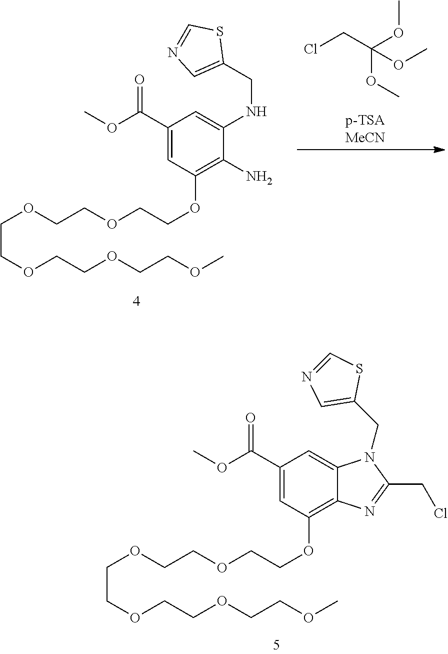

To a solution of 4 (40 mg, 77.9 μmol, 1 eq) in ACN (1 mL) was added TosOH (1.34 mg, 7.79 μmol, 0.1 eq) and 2-chloro-1,1,1-trimethoxyethane (13.2 mg, 85.7 μmol, 11.6 μL, 1.1 eq). The mixture was stirred at 60° C. for 1 h. LC-MS showed starting material was consumed completely and desired mass was detected. The reaction mixture was concentrated under reduced pressure to provide product as a colorless oil (44 mg, 76.9 μmol, 99% yield).

LCMS: Rt=1.462 min, MS cal.: 571.18, [M+H]⁺=572.2

### Preparation of Intermediate (6)

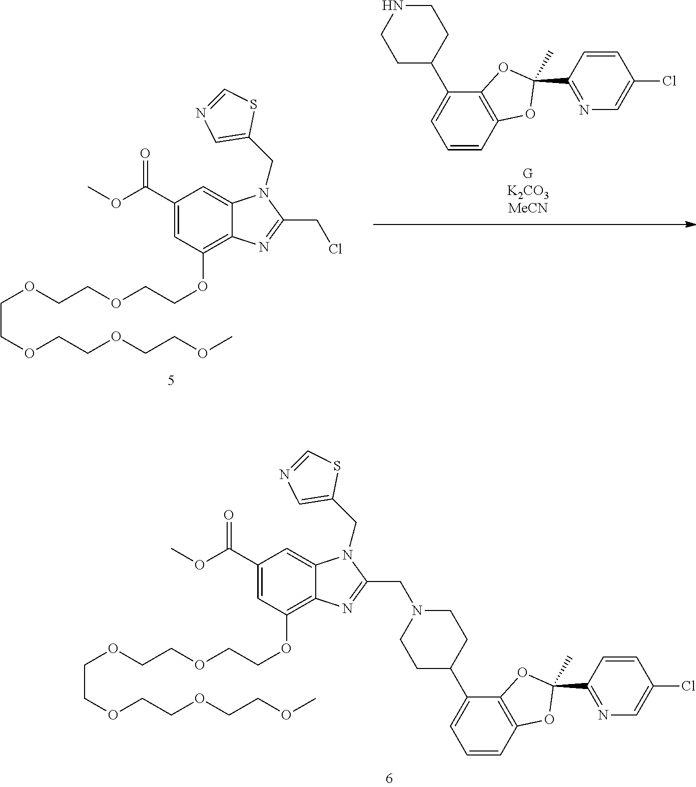 [SMILES: COCCOCCOCCOCCOCCOc1cc(C(=O)OC)cc2c1nc(CCl)n2Cc1cncs1 ; C[C@]1(c2ccc(Cl)cn2)Oc2cccc(C3CCNCC3)c2O1 ; COCCOCCOCCOCCOCCOc1cc(C(=O)OC)cc2c1nc(CN1CCC(c3cccc4c3O[C@@](C)(c3ccc(Cl)cn3)O4)CC1)n2Cc1cncs1]

To a solution of (S)-5-chloro-2-(2-methyl-4-(piperidin-4-yl)benzo[d][1,3]dioxol-2-yl)pyridine G (38.2 mg, 115 μmol, 1.5 eq), methyl 4-((2,5,8,11,14-pentaoxahexadecan-16-yl)oxy)-2-(chloromethyl)-1-(thiazol-5-ylmethyl)-1H-benzo[d]imidazole-6-carboxylate 5 (44 mg, 76.9 μmol, 1 eq) in CH₃CN (1 mL) was added K₂CO₃ (31.9 mg, 231 μmol, 3 eq). The mixture was stirred at 60° C. for 2 h. LC-MS showed starting material was consumed completely and desired mass was detected. The reaction mixture was diluted with H₂O (20 mL) and extracted with EtOAc (20 mL*3). The combined organic layers were dried over Na₂SO₄, filtered, and concentrated under reduced pressure to give a residue. The residue was purified by prep-HPLC (neutral condition; column: Waters Xbridge Prep OBD C18 150*40 mm*10 um; mobile phase: [H₂O (10 mM NH₄HCO₃)-ACN]; gradient: 40%-80% B over 8.0 min to provide product as a white solid (40 mg, 462 μmol, 60% yield).

LCMS: Rt=2.157 min, MS cal.: 865.31, [M+H]⁺=866.6

### Preparation of (S)-4-((2,5,8,11,14-Pentaoxahexadecan-16-yl)oxy)-2-((4-(2-(5-chloropyridin-2-yl)-2-methylbenzo[d][1,3]dioxol-4-yl)piperidin-1-yl)methyl)-1-(thiazol-5-ylmethyl)-1H-benzo[d]imidazole-6-carboxylic acid, Compound 4 (7)

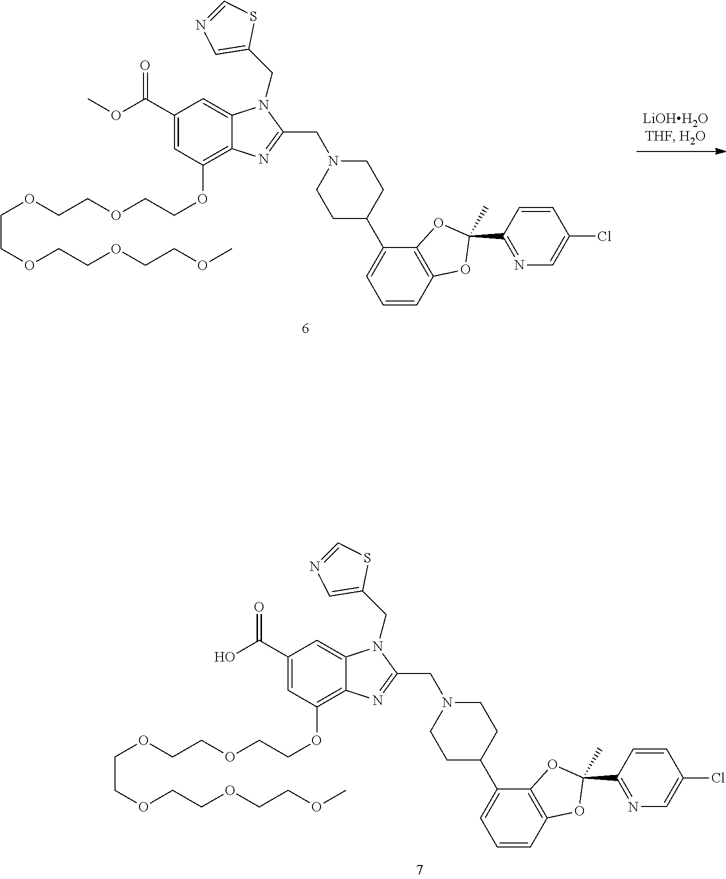 [SMILES: COCCOCCOCCOCCOCCOc1cc(C(=O)OC)cc2c1nc(CN1CCC(c3cccc4c3O[C@@](C)(c3ccc(Cl)cn3)O4)CC1)n2Cc1cncs1 ; COCCOCCOCCOCCOCCOc1cc(C(=O)O)cc2c1nc(CN1CCC(c3cccc4c3O[C@@](C)(c3ccc(Cl)cn3)O4)CC1)n2Cc1cncs1]

To a solution of methyl 4-((2,5,8,11,14-pentaoxahexadecan-16-yl)oxy)-2-(chloromethyl)-1-(thiazol-5-ylmethyl)-1H-benzo[d]imidazole-6-carboxylate 6 (35 mg, 40.4 μmol, 1 eq) in THF (0.3 mL) was added LiOH·H₂O (2.54 mg, 60.6 μmol, 1.5 eq) in H₂O (0.1 mL). The mixture was stirred at 25° C. for 12 h. LC-MS showed starting material was consumed completely and desired mass was detected. The reaction was purified directly. The residue was purified by prep-HPLC (neutral condition; column: Waters Xbridge BEH C18 100*30 mm*10 um; mobile phase: [H₂O (10 mM NH₄HCO₃)-ACN]; gradient: 25%-55% B over 8.0 min) to provide product as a white solid (24.24 mg, 28.4 μmol, 70% yield).

LCMS: Rt=1.619 min, MS cal.: 851.30, [M+H]⁺=852.3

HPLC: Rt=10.595 min,

¹H NMR (400 MHz, DMSO-d₆) δ=8.96 (s, 1H), 8.71 (d, J=2.4 Hz, 1H), 8.04-7.95 (m, 2H), 7.79 (s, 1H), 7.59 (d, J=8.4 Hz, 1H), 7.27 (s, 1H), 6.83-6.74 (m, 2H), 6.73-6.67 (m, 1H), 5.90 (s, 2H), 4.36-4.28 (m, 2H), 3.87-3.79 (m, 4H), 3.64-3.60 (m, 2H), 3.55 (m, 2H), 3.53-3.45 (m, 10H), 3.39 (m, 2H), 3.20 (s, 3H), 2.95 (m, 2H), 2.63 (m, 1H), 2.23-2.13 (m, 2H), 2.00 (s, 3H), 1.73-1.61 (m, 4H).

### Example A5. —Preparation of Compound 5. (R)-4-((2,5,8,11,14-pentaoxahexadecan-16-yl)oxy)-2-((4-(2-(5-chloropyridin-2-yl)-2-methylbenzo[d][1,3]dioxol-4-yl)piperidin-1-yl)methyl)-1-(thiazol-5-ylmethyl)-1H-benzo[d]imidazole-6-carboxylic acid (Compound 5) (3)

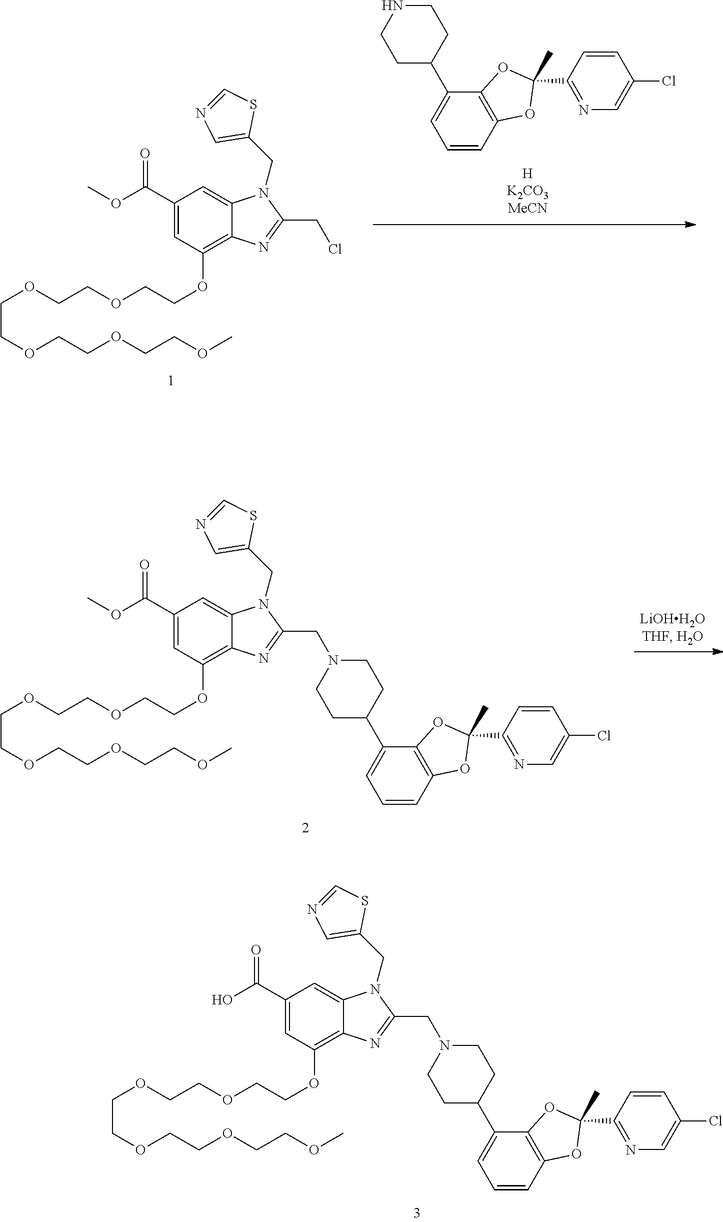 [SMILES: COCCOCCOCCOCCOCCOc1cc(C(=O)OC)cc2c1nc(CCl)n2Cc1cncs1 ; C[C@@]1(c2ccc(Cl)cn2)Oc2cccc(C3CCNCC3)c2O1 ; COCCOCCOCCOCCOCCOc1cc(C(=O)OC)cc2c1nc(CN1CCC(c3cccc4c3O[C@](C)(c3ccc(Cl)cn3)O4)CC1)n2Cc1cncs1 ; COCCOCCOCCOCCOCCOc1cc(C(=O)O)cc2c1nc(CN1CCC(c3cccc4c3O[C@](C)(c3ccc(Cl)cn3)O4)CC1)n2Cc1cncs1]

### Preparation of Intermediate (2) Methyl (R)-4-((2,5,8,11,14-pentaoxahexadecan-16-yl)oxy)-2-((4-(2-(5-chloropyridin-2-yl)-2-methylbenzo[d][1,3]dioxol-4-yl)piperidin-1-yl)methyl)-1-(thiazol-5-ylmethyl)-1H-benzo[d]imidazole-6-carboxylate

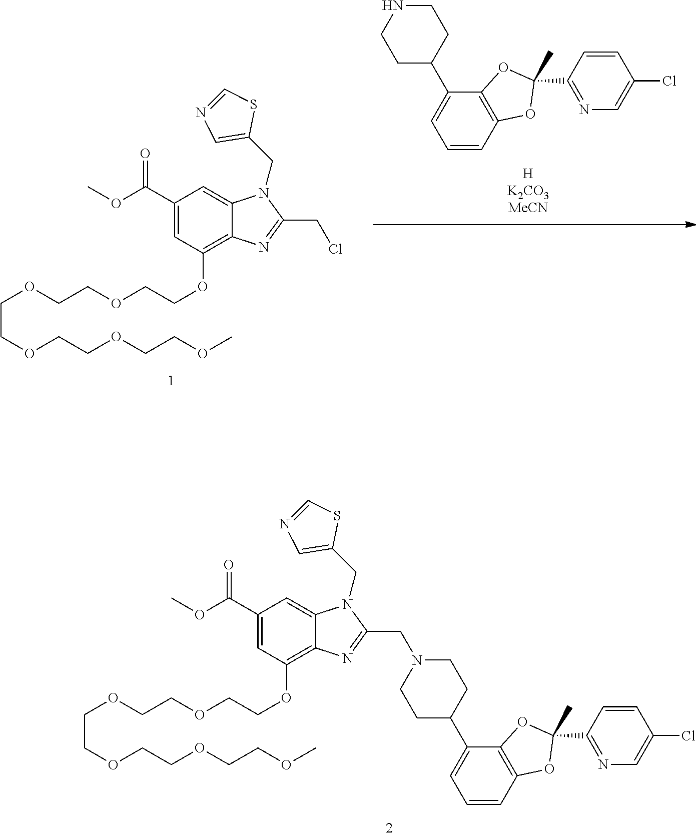 [SMILES: COCCOCCOCCOCCOCCOc1cc(C(=O)OC)cc2c1nc(CCl)n2Cc1cncs1 ; C[C@@]1(c2ccc(Cl)cn2)Oc2cccc(C3CCNCC3)c2O1 ; COCCOCCOCCOCCOCCOc1cc(C(=O)OC)cc2c1nc(CN1CCC(c3cccc4c3O[C@](C)(c3ccc(Cl)cn3)O4)CC1)n2Cc1cncs1]

A mixture of methyl 4-((2,5,8,11,14-pentaoxahexadecan-16-yl)oxy)-2-(chloromethyl)-1-(thiazol-5-ylmethyl)-1H-benzo[d]imidazole-6-carboxylate 1 (120 mg, 210 μmol, 1 eq), (R)-5-chloro-2-(2-methyl-4-(piperidin-4-yl)benzo[d][1,3]dioxol-2-yl)pyridine H (69.4 mg, 210 μmol, 1 eq) and K₂CO₃ (87 mg, 629 μmol, 3 eq) in ACN (2 mL) was degassed and purged with N₂ for 3 times, and then the mixture was stirred at 60° C. for 4 h under N₂ atmosphere. LC-MS showed starting material was consumed completely and one main peak with desired mass was detected. The residue mixture was diluted with H₂O (50 mL) and extracted with EtOAc (50 mL*3). The reaction mixture was poured into separator funnel and separated. The combined organic layers were washed with brine (50 mL), dried over Na₂SO₄, filtered, and concentrated under reduced pressure to give a residue. The residue was purified by column chromatography (SiO₂, Petroleum ether/Ethyl acetate=100/1 to 0/1, TLC-Ethyl acetate/Methanol=10/1, R_{f}=0.45). to provide product as a white solid (68 mg, 78.5 μmol, 37% yield).

LCMS: RT=2.162 min, MS cal.: 865.31, [M+H]⁺=866.6

### Preparation of (R)-4-((2,5,8,11,14-pentaoxahexadecan-16-yl)oxy)-2-((4-(2-(5-chloropyridin-2-yl)-2-methylbenzo[d][1,3]dioxol-4-yl)piperidin-1-yl)methyl)-1-(thiazol-5-ylmethyl)-1H-benzo[d]imidazole-6-carboxylic acid Compound 5 (3)

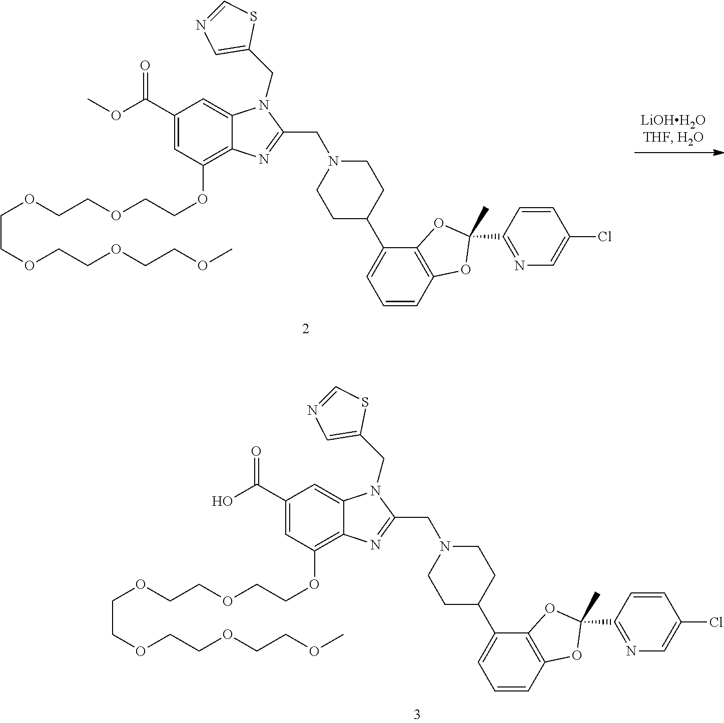 [SMILES: COCCOCCOCCOCCOCCOc1cc(C(=O)OC)cc2c1nc(CN1CCC(c3cccc4c3O[C@](C)(c3ccc(Cl)cn3)O4)CC1)n2Cc1cncs1 ; COCCOCCOCCOCCOCCOc1cc(C(=O)O)cc2c1nc(CN1CCC(c3cccc4c3O[C@](C)(c3ccc(Cl)cn3)O4)CC1)n2Cc1cncs1]

A mixture of methyl (R)-4-((2,5,8,11,14-pentaoxahexadecan-16-yl)oxy)-2-((4-(2-(5-chloropyridin-2-yl)-2-methylbenzo[d][1,3]dioxol-4-yl)piperidin-1-yl)methyl)-1-(thiazol-5-ylmethyl)-1H-benzo[d]imidazole-6-carboxylate 2 (60 mg, 69.3 μmol, 1 eq), LiOH·H₂O (4.36 mg, 104 μmol, 1.5 eq) in THF (0.7 mL) and H₂O (0.3 mL) was degassed and purged with N₂ for 3 times, and then the mixture was stirred at 25° C. for 12 h under N₂ atmosphere. LC-MS showed starting material was consumed completely and one main peak with desired mass was detected. The reaction mixture was filtered and the filtrate was concentrated. The crude product was purified by reversed-phase HPLC (column: Waters Xbridge BEH C18 100*30 mm 5 um; mobile phase: [H₂O (10 mM NH₄HCO₃)-ACN]; gradient: 22%-52% B over 8.0 min) to provide product as a white solid (26.2 mg, 30.7 μmol, 44% yield, 100% purity).

LCMS: RT=1.538 min, MS cal.: 851.30, [M+H]⁺=852.6

¹H NMR (400 MHz, METHANOL-d₄) δ=8.94 (s, 1H), 8.61 (d, J=2.0 Hz, 1H), 7.98 (s, 1H), 7.90-7.85 (m, 2H), 7.66-7.62 (d, J=8.8 Hz, 1H), 7.50-7.48 (s, 1H), 6.82-6.77 (m, 1H), 6.72-6.68 (m, 2H), 5.95 (s, 2H), 4.42-4.38 (m, 2H), 4.00-3.96 (m, 2H), 3.92 (s, 2H), 3.77-3.71 (m, 2H), 3.68-3.65 (m, 2H), 3.64-3.61 (m, 2H), 3.54 (m, 8H), 3.51-3.46 (m, 2H), 3.31-3.30 (m, 3H), 3.02 (m, 2H), 2.77-2.67 (m, 1H), 2.30 (m, 2H), 2.02 (s, 3H), 1.84-1.73 (m, 4H).

### Example A6. —Preparation of Compound 6. (S)-2-((4-(2-(5-chloropyridin-2-yl)-2-methylbenzo[d][1,3]dioxol-4-yl)piperidin-1-yl)methyl)-4-cyclopropoxy-1-(thiazol-5-ylmethyl)-1H-benzo[d]imidazole-6-carboxylic acid (Compound 6) (7)

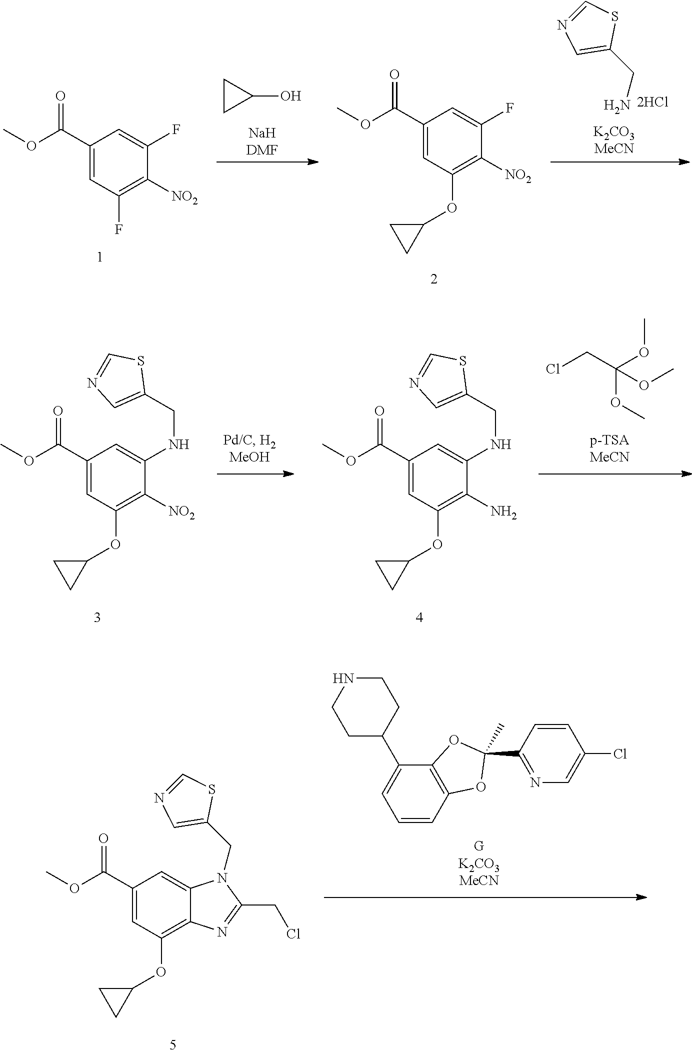

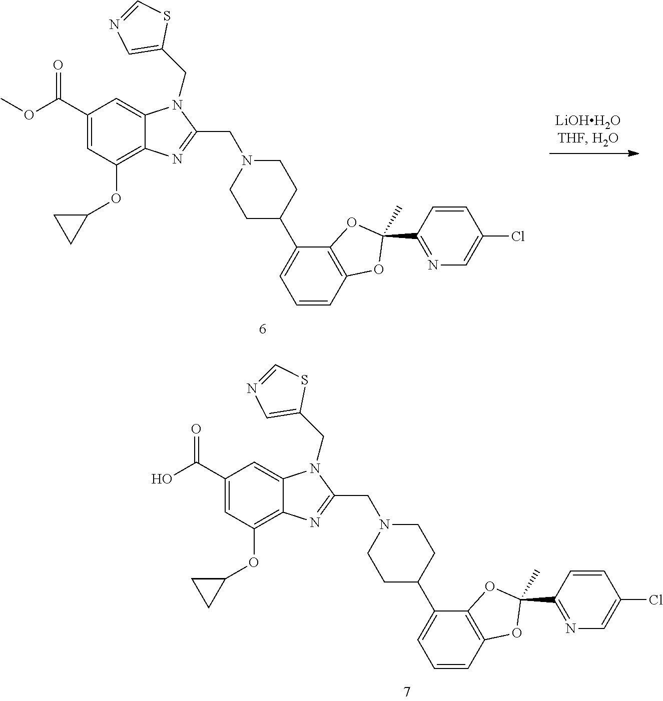 [SMILES: COC(=O)c1cc(OC2CC2)c2nc(CN3CCC(c4cccc5c4O[C@@](C)(c4ccc(Cl)cn4)O5)CC3)n(Cc3cncs3)c2c1 ; C[C@]1(c2ccc(Cl)cn2)Oc2cccc(C3CCN(Cc4nc5c(OC6CC6)cc(C(=O)O)cc5n4Cc4cncs4)CC3)c2O1]

### Preparation of Intermediate (2) methyl 3-cyclopropoxy-5-fluoro-4-nitrobenzoate

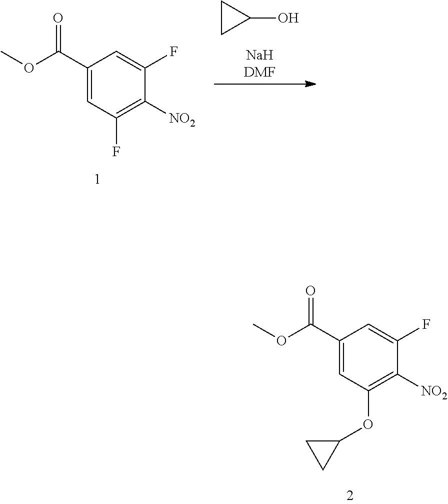

THF (7 mL) was charged to the 50 mL three-necked round bottom flask and then cyclopropanol (452 mg, 7.78 mmol, 1.3 eq) was added at 20° C. At 0° C. (inner temperature), NaH (263 mg, 6.59 mmol, 60%, 1.1 eq) was added to the reaction mixture at 0° C. within 2 min. After the addition, the mixture was stirred at 0° C. for 0.5 h. And then, methyl 3,5-difluoro-4-nitrobenzoate 1 (1.3 g, 5.99 mmol, 1 eq) in THF (7 mL) was added to the reaction mixture at 0° C. within 2 min. After the addition, the mixture was stirred at 0° C. for 2 h. TLC (Petroleum ether/Ethyl acetate=3/1) indicated starting material was consumed completely and one new spot formed. The reaction mixture was diluted with H₂O (10 mL) and extracted with EtOAc (20 mL*5). The combined organic layers were washed with brine (10 mL), dried over Na₂SO₄, filtered, and concentrated under reduced pressure to give a residue. The residue was purified by column chromatography (SiO₂, Petroleum ether/Ethyl acetate=100/1 to 0/1) to provide product as a white solid (900 mg, 3.53 mmol, 59% yield).

¹H NMR (400 MHz, CHLOROFORM-d) δ=7.85 (t, J=1.6 Hz, 1H), 7.51 (dd, J=1.6, 9.6 Hz, 1H), 3.99-3.97 (s, 3H), 3.97-3.88 (m, 1H), 0.93-0.86 (m, 4H)

### Preparation of Intermediate (3) Methyl 3-cyclopropoxy-4-nitro-5-((thiazol-5-ylmethyl)amino)benzoate

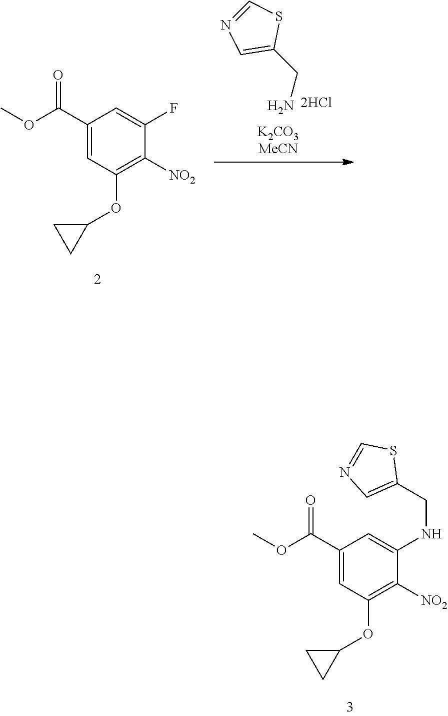

A mixture of methyl 3-cyclopropoxy-5-fluoro-4-nitrobenzoate 1 (900 mg, 3.53 mmol, 1 eq), thiazol-5-ylmethanamine (1.98 g, 10.6 mmol, 3 eq, 2HCl) and K₂CO₃ (1.46 g, 10.6 mmol, 3 eq) in DMF (5 mL) and CH₃CN (10 mL) was degassed and purged with N₂ for 3 times, and then the mixture was stirred at 60° C. for 48 h under N₂ atmosphere. LCMS showed ˜34% of desired compound was detected. The reaction mixture was diluted with H₂O (10 mL) and extracted with EtOAc (15 mL*3). The combined organic layers were washed with brine (10 mL), dried over Na₂SO₄, filtered, and concentrated under reduced pressure to give a residue. The residue was purified by column chromatography (SiO₂, Petroleum ether/Ethyl acetate=100/1 to 0/1) to provide product as a white solid (300 mg, 859 μmol, 24% yield).

LCMS: RT=1.758 min, MS cal.: 349.07, [M+H]⁺=350.1

¹H NMR (400 MHz, CHLOROFORM-d) δ=8.77 (s, 1H), 7.86 (s, 1H), 7.37 (d, J=1.6 Hz, 1H), 7.15 (d, J=1.6 Hz, 1H), 6.07-5.99 (m, 1H), 4.70 (d, J=5.6 Hz, 2H), 3.93 (s, 3H), 3.92-3.89 (m, 1H), 0.88-0.84 (m, 4H).

### Preparation of Intermediate (4), Methyl 4-amino-3-cyclopropoxy-5-((thiazol-5-ylmethyl)amino)benzoate

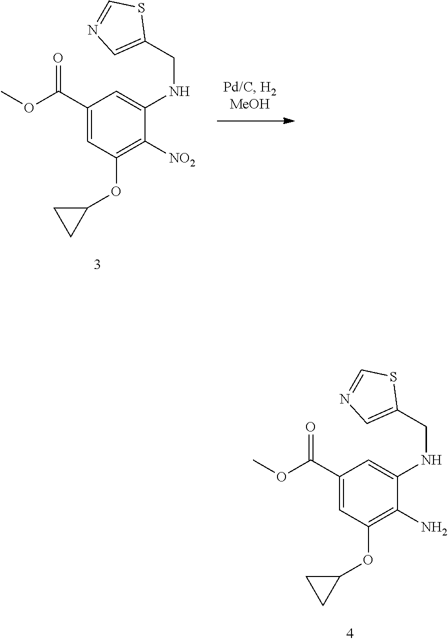 [SMILES: COC(=O)c1cc(NCc2cncs2)c([N+](=O)[O-])c(OC2CC2)c1 ; COC(=O)c1cc(NCc2cncs2)c(N)c(OC2CC2)c1]

A mixture of methyl 3-cyclopropoxy-4-nitro-5-((thiazol-5-ylmethyl)amino)benzoate 3 (300 mg, 859 μmol, 1 eq) and Pd/C (914 mg, 10% on carbon) in MeOH (5 mL) was degassed and purged with H₂ for 3 times, and then the mixture was stirred at 20° C. for 2 h under H₂ atmosphere. LCMS showed the starting material was consumed completely. The suspension was filtered through a pad of celite and the filter cake was washed with MeOH (8 mL*5). The combined filtrates were concentrated to provide product as a yellow solid (260 mg, 814 μmol, 95% yield).

LCMS: RT=0.403 min, MS cal.: 319.10, [M+H]⁺=320.2

### Preparation of Intermediate (5), Methyl 2-(chloromethyl)-4-cyclopropoxy-1-(thiazol-5-ylmethyl)-1H-benzo[d]imidazole-6-carboxylate

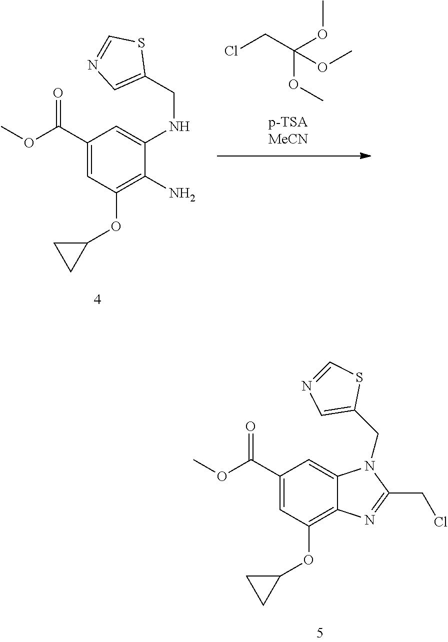

A mixture of Methyl 4-amino-3-cyclopropoxy-5-((thiazol-5-ylmethyl)amino)benzoate 4 (260 mg, 814 μmol, 1 eq), 2-chloro-1,1,1-trimethoxyethane (378 mg, 2.44 mmol, 329 μL, 3 eq), p-TSA (70.1 mg, 407 μmol, 0.5 eq) in CH₃CN (3 mL) was degassed and purged with N₂ for 3 times, and then the mixture was stirred at 60° C. for 2 h under N₂ atmosphere. LCMS showed the starting material was consumed completely. The reaction mixture was concentrated under reduced pressure to provide product as a white solid (390 mg, crude).

LCMS: RT=0.416 min, MS cal.: 377.06, [M+H]⁺=378.2

### Preparation of Intermediate (6) Methyl (S)-2-((4-(2-(5-chloropyridin-2-yl)-2-methylbenzo[d][1,3]dioxol-4-yl)piperidin-1-yl)methyl)-4-cyclopropoxy-1-(thiazol-5-ylmethyl)-1H-benzo[d]imidazole-6-carboxylate

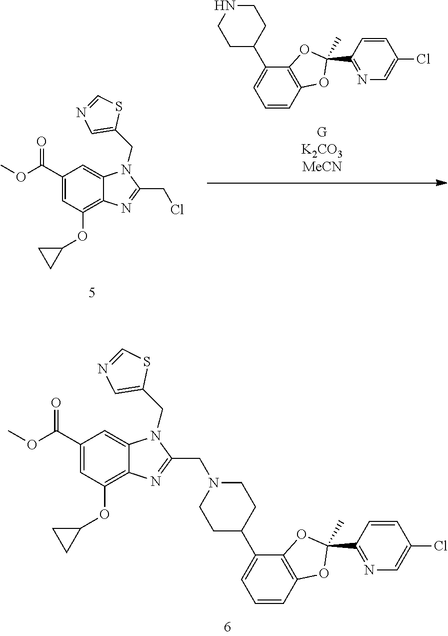 [SMILES: COC(=O)c1cc(OC2CC2)c2nc(CCl)n(Cc3cncs3)c2c1 ; C[C@]1(c2ccc(Cl)cn2)Oc2cccc(C3CCNCC3)c2O1 ; COC(=O)c1cc(OC2CC2)c2nc(CN3CCC(c4cccc5c4O[C@@](C)(c4ccc(Cl)cn4)O5)CC3)n(Cc3cncs3)c2c1]

A mixture of methyl 2-(chloromethyl)-4-cyclopropoxy-1-(thiazol-5-ylmethyl)-1H-benzo[d]imidazole-6-carboxylate 5 (100 mg, 265 μmol, 1 eq), (S)-5-chloro-2-(2-methyl-4-(piperidin-4-yl)benzo[d][1,3]dioxol-2-yl)pyridine G (105 mg, 318 μmol, 1.2 eq) and K₂CO₃ (110 mg, 794 μmol, 3 eq) in CH₃CN (3 mL) was degassed and purged with N₂ for 3 times, and then the mixture was stirred at 60° C. for 2 h under N₂ atmosphere. LCMS showed the starting material was consumed completely. The reaction mixture was diluted with H₂O (10 mL) and extracted with EtOAc (10 mL*3). The combined organic layers were washed with brine (15 mL), dried over Na₂SO₄, filtered, and concentrated under reduced pressure to give a residue. The residue was purified by column chromatography (SiO₂, Petroleum ether/Ethyl acetate=100/1 to 0/1) to provide product as a white solid (50 mg, 74.4 μmol, 28% yield).

LCMS: RT=2.398 min, MS cal.: 671.20, [M+H]⁺=672.1

¹H NMR (400 MHz, CHLOROFORM-d) δ=8.74 (s, 1H), 8.65-8.59 (s, 1H), 7.90-7.82 (s, 1H), 7.81-7.74 (m, 2H), 7.70-7.65 (m, 1H), 7.61-7.52 (m, 1H), 6.84-6.75 (m, 1H), 6.74-6.64 (m, 2H), 5.89 (s, 2H), 4.03-3.93 (m, 4H), 3.89 (s, 2H), 3.02-2.90 (m, 2H), 2.81-2.68 (m, 1H), 2.38-2.24 (m, 2H), 2.07-2.03 (m, 3H), 1.90-1.66 (m, 4H), 1.01-0.86 (m, 4H).

### Preparation of (S)-2-((4-(2-(5-chloropyridin-2-yl)-2-methylbenzo[d][1,3]dioxol-4-yl)piperidin-1-yl)methyl)-4-cyclopropoxy-1-(thiazol-5-ylmethyl)-1H-benzo[d]imidazole-6-carboxylic acid, Compound 6 (7)

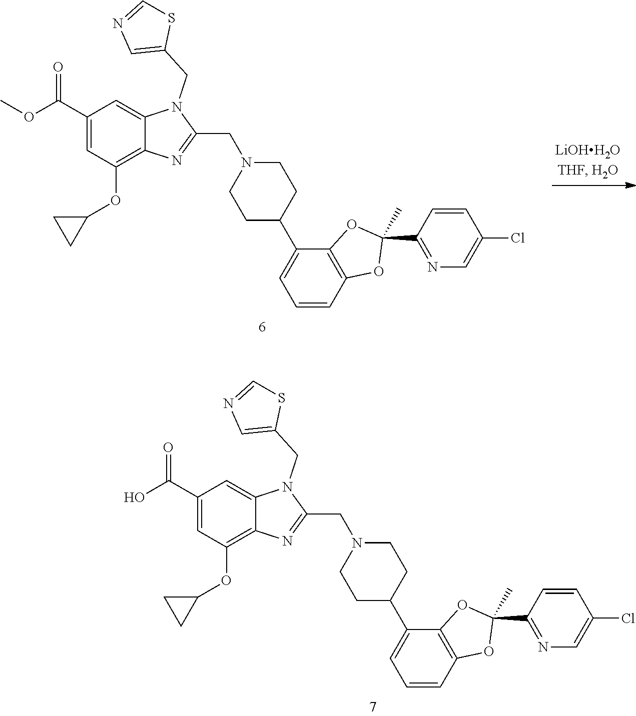 [SMILES: COC(=O)c1cc(OC2CC2)c2nc(CN3CCC(c4cccc5c4O[C@@](C)(c4ccc(Cl)cn4)O5)CC3)n(Cc3cncs3)c2c1 ; C[C@]1(c2ccc(Cl)cn2)Oc2cccc(C3CCN(Cc4nc5c(OC6CC6)cc(C(=O)O)cc5n4Cc4cncs4)CC3)c2O1]

A mixture of methyl (S)-2-((4-(2-(5-chloropyridin-2-yl)-2-methylbenzo[d][1,3]dioxol-4-yl)piperidin-1-yl)methyl)-4-cyclopropoxy-1-(thiazol-5-ylmethyl)-1H-benzo[d]imidazole-6-carboxylate 6 (70 mg, 104 μmol, 1 eq), LiOH·H₂O (6.55 mg, 156 μmol, 1.5 eq) in THF (0.7 mL) and H₂O (0.3 mL) was degassed and purged with N₂ for 3 times, and then the mixture was stirred at 25° C. for 12 h under N₂ atmosphere. LCMS showed the starting material was consumed completely. The reaction mixture was concentrated under reduced pressure to remove solvent. The residue was purified by prep-HPLC (column: Waters Xbridge Prep OBD C18 150*40 mm*10 um; mobile phase: [H₂O (10 mM NH₄HCO₃)—CH₃CN]; gradient: 15%-75% B over 8.0 min) to provide product as a white solid (24.66 mg, 37.5 μmol, 36% yield).

LCMS: RT=2.733 min, MS cal.: 658.2, [M+H]⁺=658.0

HPLC: RT=11.057 min, purity: 97.08%

¹H NMR (400 MHz, METHANOL-d4) δ=8.93 (s, 1H), 8.60 (s, 1H), 7.98-7.95 (s, 1H), 7.89-7.84 (m, 2H), 7.78 (s, 1H), 7.66-7.60 (m, 1H), 6.82-6.75 (m, 1H), 6.72-6.66 (m, 2H), 5.94 (m, 2H), 4.09-3.99 (m, 1H), 3.92 (s, 2H), 3.05-2.93 (m, 2H), 2.77-2.64 (m, 1H), 2.34-2.23 (m, 2H), 2.01 (s, 3H), 1.84-1.72 (m, 4H), 0.93-0.84 (m, 4H).

### Example A7. —Preparation of Compound 7. (R)-2-((4-(2-(5-chloropyridin-2-yl)-2-methylbenzo[d][1,3]dioxol-4-yl)piperidin-1-yl)methyl)-4-cyclopropoxy-1-(thiazol-5-ylmethyl)-1H-benzo[d]imidazole-6-carboxylic acid (Compound 7) (3)

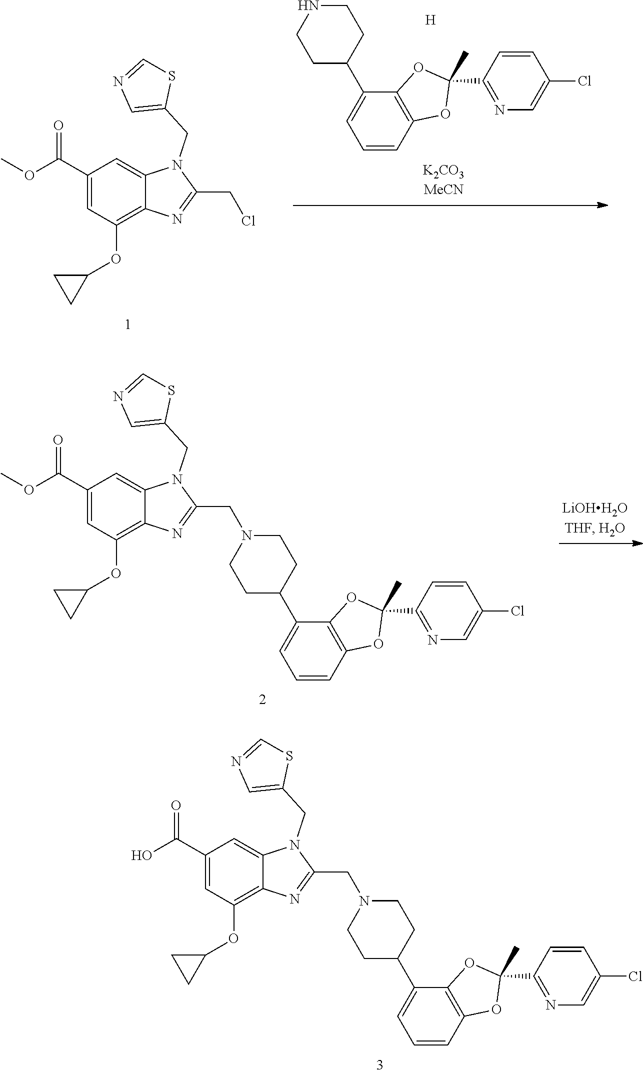

### Preparation of Intermediate (2), methyl (R)-2-((4-(2-(5-chloropyridin-2-yl)-2-methylbenzo[d][1,3]dioxol-4-yl)piperidin-1-yl)methyl)-4-cyclopropoxy-1-(thiazol-5-ylmethyl)-1H-benzo[d]imidazole-6-carboxylate

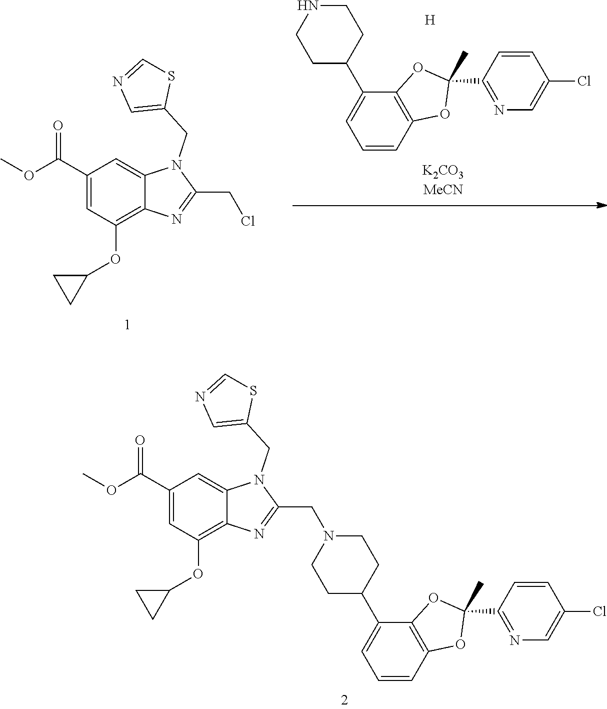

A mixture of methyl 2-(chloromethyl)-4-cyclopropoxy-1-(thiazol-5-ylmethyl)-1H-benzo[d]imidazole-6-carboxylate, 1 (200 mg, 529 μmol, 1 eq), (R)-5-chloro-2-(2-methyl-4-(piperidin-4-yl)benzo[d][1,3]dioxol-2-yl)pyridine, H (210.12 mg, 635.18 μmol, 1.2 eq) and K₂CO₃ (658 mg, 4.76 mmol, 9 eq) in CH₃CN (2 mL) was degassed and purged with N₂ for 3 times, and then the mixture was stirred at 60° C. for 2 hrs under N₂ atmosphere. LCMS showed starting material was consumed completely and one main peak with desired mass was detected. The reaction mixture was partitioned between DCM (15 mL*3) mL and water (5 mL). The organic phase was separated, dried over Na₂SO₄, filtered, and concentrated under reduced pressure to give a residue. The residue was purified by column chromatography (SiO₂, Petroleum ether/Ethyl acetate=100/1 to 1/1) to provide product as a yellow solid (160 mg, 238 μmol, 45% yield).

LCMS: RT=2.237 min, MS cal. 671.20, [M+H]⁺=672.2

¹H NMR (400 MHz, CHLOROFORM-d) δ=8.75 (s, 1H), 8.62 (s, 1H), 7.85 (s, 1H), 7.80-7.73 (m, 2H), 7.68 (m, 1H), 7.56 (m, 1H), 6.82-6.75 (m, 1H), 6.74-6.63 (m, 2H), 5.89 (s, 2H), 4.02-3.93 (m, 4H), 3.91-3.85 (s, 2H), 3.05-2.89 (m, 2H), 2.81-2.69 (m, 1H), 2.39-2.22 (m, 2H), 2.05 (s, 3H), 1.90-1.68 (m, 4H), 1.00-0.95 (m, 2H), 0.89 (m, 2H).

### Preparation of (R)-2-((4-(2-(5-chloropyridin-2-yl)-2-methylbenzo[d][1,3]dioxol-4-yl)piperidin-1-yl)methyl)-4-cyclopropoxy-1-(thiazol-5-ylmethyl)-1H-benzo[d]imidazole-6-carboxylic acid Compound 7 (3)

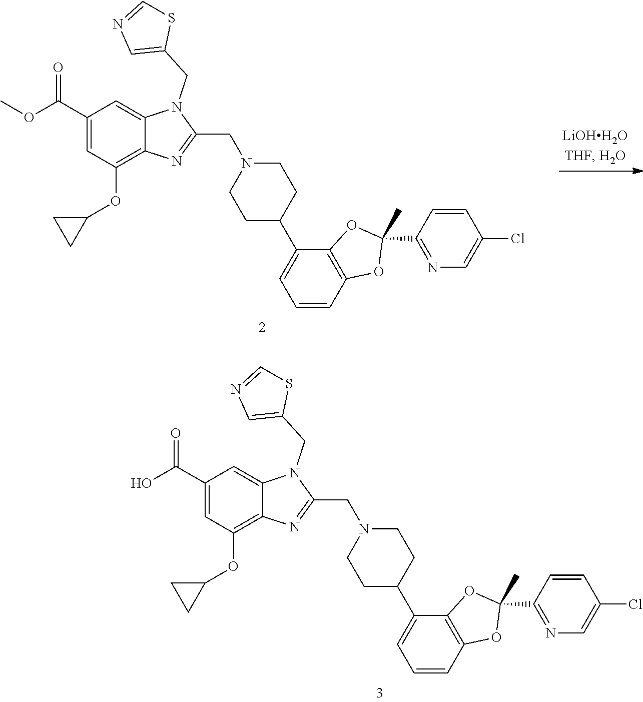 [SMILES: COC(=O)c1cc(OC2CC2)c2nc(CN3CCC(c4cccc5c4O[C@](C)(c4ccc(Cl)cn4)O5)CC3)n(Cc3cncs3)c2c1 ; C[C@@]1(c2ccc(Cl)cn2)Oc2cccc(C3CCN(Cc4nc5c(OC6CC6)cc(C(=O)O)cc5n4Cc4cncs4)CC3)c2O1]

A mixture of methyl (R)-2-((4-(2-(5-chloropyridin-2-yl)-2-methylbenzo[d][1,3]dioxol-4-yl)piperidin-1-yl)methyl)-4-cyclopropoxy-1-(thiazol-5-ylmethyl)-1H-benzo[d]imidazole-6-carboxylate, 2 (0.1 g, 149 μmol, 1 eq) and LiOH·H₂O (9.36 mg, 223 μmol, 1.5 eq) in THF (0.7 mL) and H₂O (0.3 mL) was degassed and purged with N₂ for 3 times, and then the mixture was stirred at 25° C. for 12 h under N₂ atmosphere. LCMS showed starting material was consumed completely and one main peak with desired mass was detected. The reaction mixture was filtered and concentrated under reduced pressure to give a residue. The residue was purified by prep-HPLC (column: Waters Xbridge Prep OBD C18 150*40 mm*10 um; mobile phase: [H₂O (10 mM NH₄HCO₃)—CH₃CN]; gradient: 30%-60% B over 8.0 min) to provide product as a white solid (32.9 mg, 50.1 μmol, 34% yield).

HPLC: RT=11.088 min, purity: 97.13%

LCMS: RT=2.696 min, MS cal. 657.18, [M+H]⁺=658.2

1H NMR (400 MHz, METHANOL-d₄) δ=8.93 (s, 1H), 8.59 (s, 1H), 7.96 (s, 1H), 7.89-7.83 (m, 2H), 7.78 (s, 1H), 7.62 (d, J=8.4 Hz, 1H), 6.82-6.73 (m, 1H), 6.72-6.65 (m, 2H), 5.94 (s, 2H), 4.02 (m, 1H), 3.89 (s, 2H), 2.99 (m, 2H), 2.69 (m, 1H), 2.33-2.22 (m, 2H), 2.00 (s, 3H), 1.83-1.71 (m, 4H), 0.93-0.87 (m, 2H), 0.87-0.82 (m, 2H).

### Example A8. —Preparation of Compound 8. (S)-2-((4-(6-((4-Cyano-2-fluorobenzyl)oxy)pyridin-2-yl)piperidin-1-yl)methyl)-4-methoxy-1-(oxetan-2-ylmethyl)-1H-benzo[d]imidazole-6-carboxylic acid (Compound 8) (6)

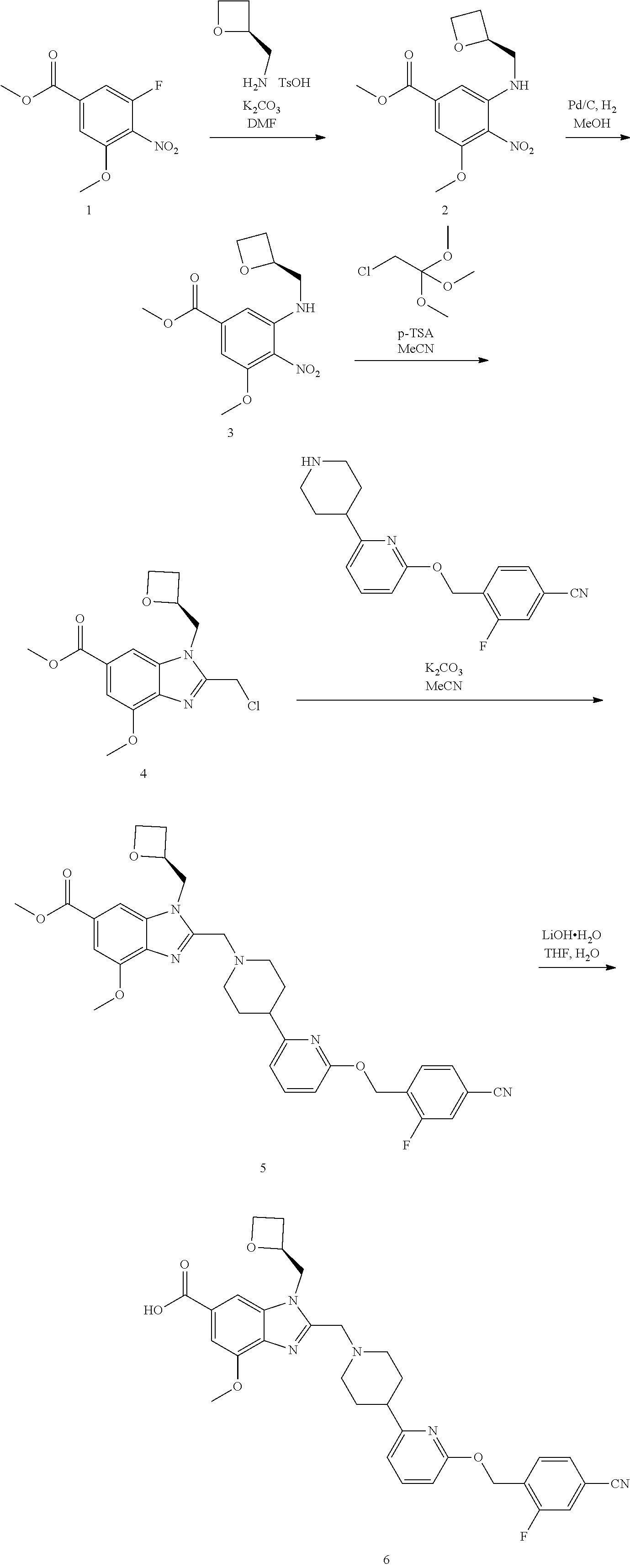

### Preparation of Intermediate Methyl (S)-3-methoxy-4-nitro-5-((oxetan-2-ylmethyl)amino)benzoate (2)

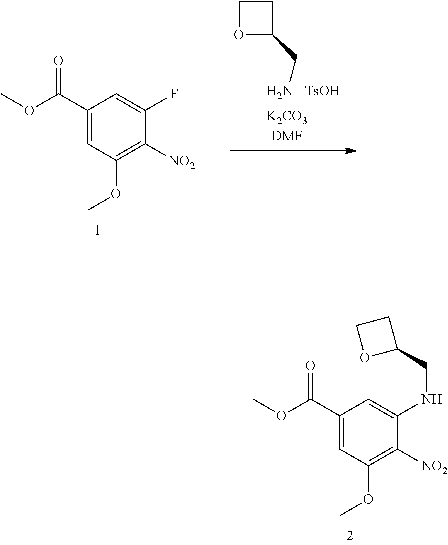

A mixture of methyl 3-fluoro-5-methoxy-4-nitrobenzoate (1) (1.3 g, 5.67 mmol, 1 eq), (S)-oxetan-2-ylmethanamine (1.77 g, 6.81 mmol, 1.2 eq, p-TSA salt) and K₂CO₃ (2.74 g, 19.85 mmol, 3.5 eq) in DMF (10 mL) was degassed and purged with N₂ for 3 times, and then the mixture was stirred at 60° C. for 12 h under N₂ atmosphere. TLC indicated starting material was consumed completely and one new spot formed. The mixture was diluted with H₂O (10 mL) and extracted with EtOAc (10 mL*3). The combined organic layers were dried over Na₂SO₄, filtered and the filtrate was concentrated to give crude product. The residue was purified by column chromatography (SiO₂, Petroleum ether/Ethyl acetate=10/1 to 3/1) to provide product as a yellow oil (1.4 g, 4.73 mmol, 83% yield).

LCMS: RT=0.441 min, MS cal.: 296.28, [M+H]⁺=297.2

¹H NMR (400 MHz, CHLOROFORM-d) δ=7.14 (d, J=1.2 Hz, 1H), 6.96 (d, J=1.2 Hz, 1H), 6.31-6.05 (br s, 1H), 5.14-5.05 (m, 1H), 4.77-4.68 (m, 1H), 4.58 (m, 1H), 3.94 (s, 6H), 3.49 (d, J=4.4 Hz, 2H), 2.78-2.67 (m, 1H), 2.63-2.50 (m, 1H).

### Preparation of Intermediate (3) Methyl (S)-4-amino-3-methoxy-5-((oxetan-2-ylmethyl)amino)benzoate

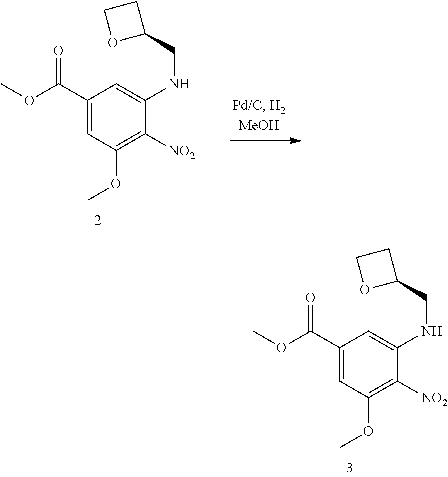 [SMILES: COC(=O)c1cc(NC[C@@H]2CCO2)c([N+](=O)[O-])c(OC)c1 ; COC(=O)c1cc(NC[C@@H]2CCO2)c([N+](=O)[O-])c(OC)c1]

To a solution of methyl (S)-3-methoxy-4-nitro-5-((oxetan-2-ylmethyl)amino)benzoate 2 (1.45 g, 4.89 mmol, 1 eq) in MeOH (10 mL) was added Pd/C (400 mg, 10%) under N₂. The suspension was degassed under vacuum and purged with H₂ several times. The mixture was stirred under H₂ (15 psi) at 20° C. for 2 h. LC-MS showed starting material was consumed completely and desired mass was detected. The suspension was filtered and the filter cake was washed with MeOH (30 mL×2). The combined filtrates were concentrated to dryness to provide product as a white solid (1.3 g, 4.88 mmol, 99% yield).

LCMS: RT=0.598 min, MS cal.: 266.30, [M+H]⁺=267.0

¹H NMR (400 MHz, METHANOL-d₄) δ=7.13 (s, 1H), 7.11 (s, 1H), 5.17-5.05 (m, 1H), 4.76-4.71 (m, 1H), 4.66-4.57 (m, 1H), 3.89 (s, 3H), 3.86 (s, 3H), 3.51-3.44 (m, 1H), 3.42-3.36 (m, 1H), 2.83-2.68 (m, 1H), 2.60 (m, 1H).

### Preparation of Intermediate (4) Methyl (S)-2-(chloromethyl)-4-methoxy-1-(oxetan-2-ylmethyl)-1H-benzo[d]imidazole-6-carboxylate

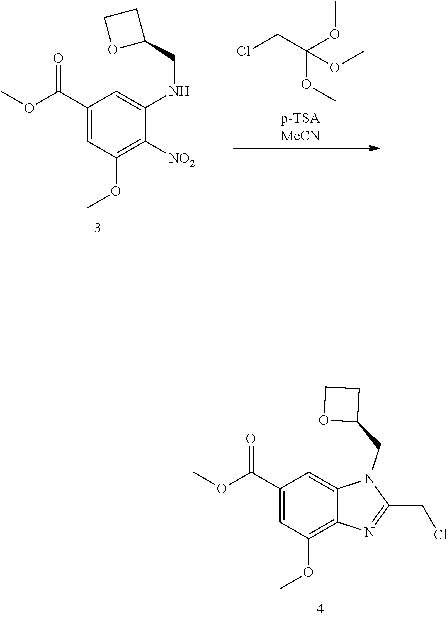

To a solution of compound methyl (S)-4-amino-3-methoxy-5-((oxetan-2-ylmethyl)amino)benzoate 3 (1.3 g, 4.88 mmol, 1 eq), 2-chloro-1,1,1-trimethoxyethane (830 mg, 5.37 mmol, 724 μL, 1.1 eq) and p-TSA (42.0 mg, 244 μmol, 0.05 eq) in CH₃CN (15 mL) was degassed and purged with N₂ for 3 times, and then the mixture was stirred at 60° C. for 2 h under N₂ atmosphere. LC-MS showed starting material was consumed completely and desired mass was detected. The reaction mixture was concentrated to dryness to provide product as a white solid (1.3 g, 4.00 mmol, 82.00% yield).

LCMS: RT=0.417 min, MS cal.: 324.76, [M+H]⁺=325.3

¹H NMR (400 MHz, CHLOROFORM-d) δ=7.64-7.58 (m, 1H), 7.29-7.25 (m, 1H), 5.13-5.02 (m, 1H), 4.95-4.83 (m, 2H), 4.52-4.31 (m, 3H), 4.22-4.11 (m, 1H), 3.94 (s, 3H), 3.85-3.76 (m, 3H), 2.60 (dtd, J=6.1, 8.0, 11.5 Hz, 1H), 2.33-2.17 (m, 1H)

### Preparation of Intermediate (5) Methyl (S)-2-((4-(6-((4-cyano-2-fluorobenzyl)oxy)pyridin-2-yl)piperidin-1-yl)methyl)-4-methoxy-1-(oxetan-2-ylmethyl)-1H-benzo[d]imidazole-6-carboxylate

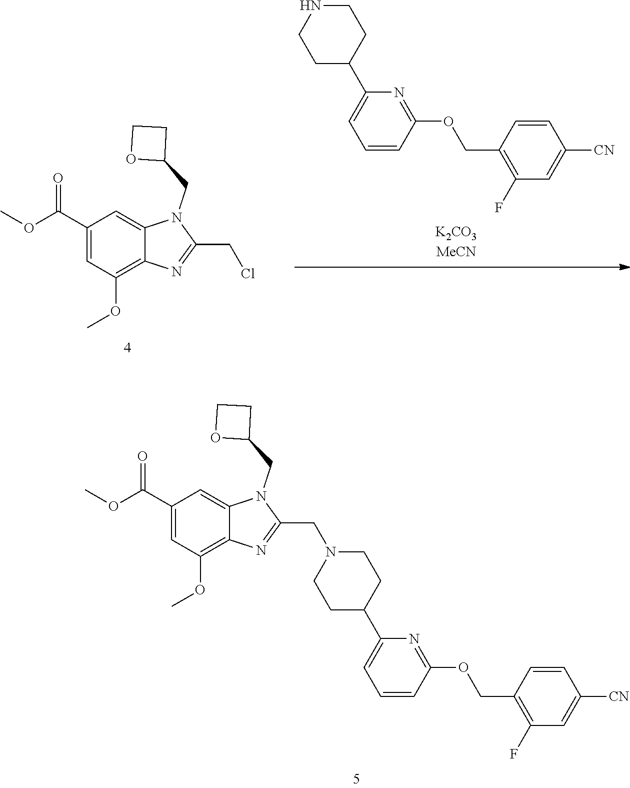 [SMILES: COC(=O)c1cc(OC)c2nc(CCl)n(C[C@@H]3CCO3)c2c1 ; N#Cc1ccc(COc2cccc(C3CCNCC3)n2)c(F)c1 ; COC(=O)c1cc(OC)c2nc(CN3CCC(c4cccc(OCc5ccc(C#N)cc5F)n4)CC3)n(C[C@@H]3CCO3)c2c1]

A mixture of methyl (S)-2-(chloromethyl)-4-methoxy-1-(oxetan-2-ylmethyl)-1H-benzo[d]imidazole-6-carboxylate 4 (131 mg, 308 μmol, 1 eq), 3-fluoro-4-(((6-(piperidin-4-yl)pyridin-2-yl)oxy)methyl)benzonitrile (100 mg, 308 μmol, 1 eq) and K₂CO₃ (42.6 mg, 308 μmol, 1 eq) in CH₃CN (2 mL) was degassed and purged with N₂ for 3 times, and then the mixture was stirred at 60° C. for 2 h under N₂ atmosphere. LCMS showed the starting material was consumed completely. The reaction mixture was concentrated under reduced pressure to remove solvent. The residue was purified by column chromatography (SiO₂, Petroleum ether/Ethyl acetate=50/1 to 1/1). The reaction mixture was concentrated to give a residue. Product was obtained as a white solid (115 mg, 192 μmol, 62.3% yield).

LCMS: RT=0.593 min, MS cal.: 599.66, [M+H]⁺=600.5

### Preparation of (S)-2-((4-(6-((4-Cyano-2-fluorobenzyl)oxy)pyridin-2-yl)piperidin-1-yl)methyl)-4-methoxy-1-(oxetan-2-ylmethyl)-1H-benzo[d]imidazole-6-carboxylic acid, Compound 8 (6)

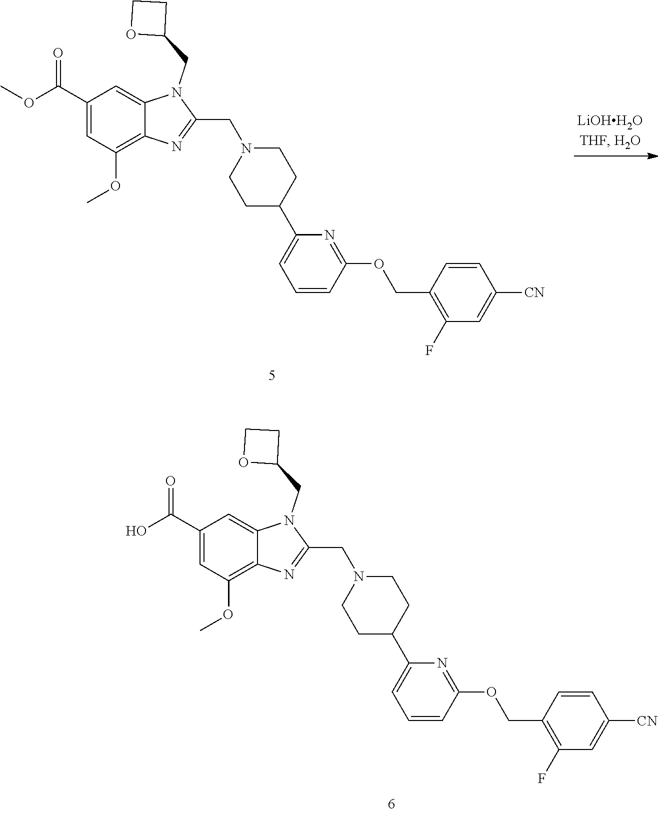 [SMILES: COC(=O)c1cc(OC)c2nc(CN3CCC(c4cccc(OCc5ccc(C#N)cc5F)n4)CC3)n(C[C@@H]3CCO3)c2c1 ; COc1cc(C(=O)O)cc2c1nc(CN1CCC(c3cccc(OCc4ccc(C#N)cc4F)n3)CC1)n2C[C@@H]1CCO1]

A mixture of methyl (S)-2-((4-(6-((4-cyano-2-fluorobenzyl)oxy)pyridin-2-yl)piperidin-1-yl)methyl)-4-methoxy-1-(oxetan-2-ylmethyl)-1H-benzo[d]imidazole-6-carboxylate 5 (130 mg, 217 μmol, 1 eq) in THF (0.7 mL) was added LiOH·H₂O (11.8 mg, 282 μmol, 1.3 eq) in H₂O (0.3 mL) was degassed and purged with N₂ for 3 times, and then the mixture was stirred at 25° C. for 12 h under N₂ atmosphere. LCMS showed the starting material was consumed completely. The residue was purified by prep-HPLC (Column: Waters Xbridge Prep OBD C18 150*40 mm*10 um; mobile phase: [NH₄HCO₃-ACN]; gradient: 20%-60% B over 8.0 min) to provide product as a white solid (31.4 mg, 53.7 μmol, 28% yield).

LC MS: RT=1.493 min, MS cal.: 585.64, [M+H]⁺=586.3

HPLC: RT=8.336 min, purity: 98.00%

¹H NMR (400 MHz, METHANOL-d₄) δ=7.92 (s, 1H), 7.70-7.63 (m, 1H), 7.62-7.49 (m, 3H), 7.45 (s, 1H), 6.84 (d, J=7.2 Hz, 1H), 6.68 (d, J=8.0 Hz, 1H), 5.51 (s, 2H), 5.26 (m, 1H), 4.82 (m, 1H), 4.75-4.57 (m, 2H), 4.46 (m, 1H), 4.12-3.93 (m, 5H), 3.17-3.07 (m, 1H), 3.11 (m, 1H), 3.02 (m, 1H), 2.85-2.72 (m, 1H), 2.71-2.60 (m, 1H), 2.58-2.47 (m, 1H), 2.46-2.30 (m, 2H), 1.92-1.76 (m, 4H)

### Example A9. —Preparation of Compound 9. (S)-2-((4-(6-((2-Fluoro-4-((methoxyimino)methyl)benzyl)oxy)pyridin-2-yl)piperidin-1-yl)methyl)-4-methoxy-1-(oxetan-2-ylmethyl)-1H-benzo[d]imidazole-6-carboxylic acid (Compound 9) (6)

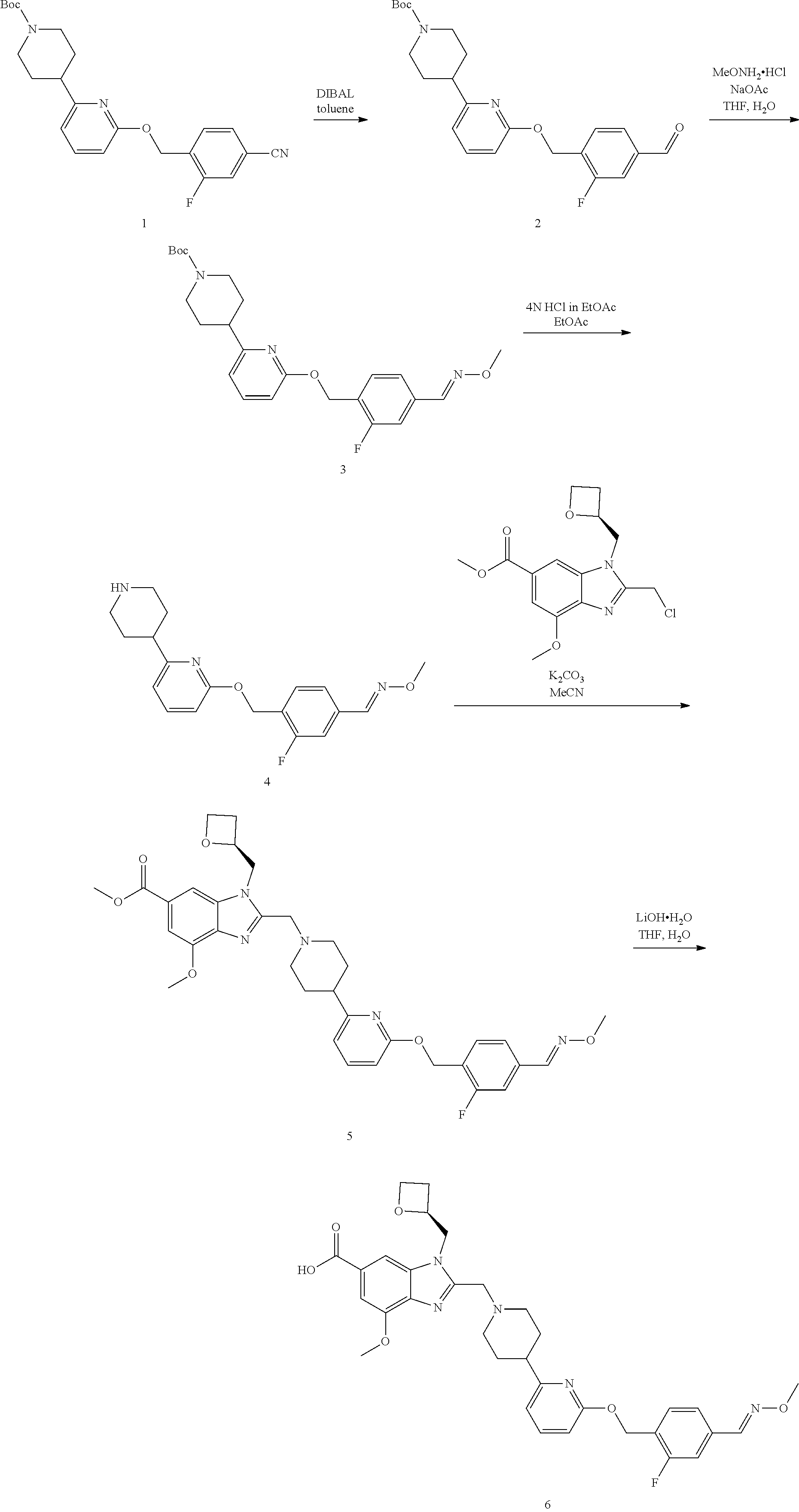

### Preparation of Intermediate (2) tert-Butyl 4-(6-((2-fluoro-4-formylbenzyl)oxy)pyridin-2-yl)piperidine-1-carboxylate

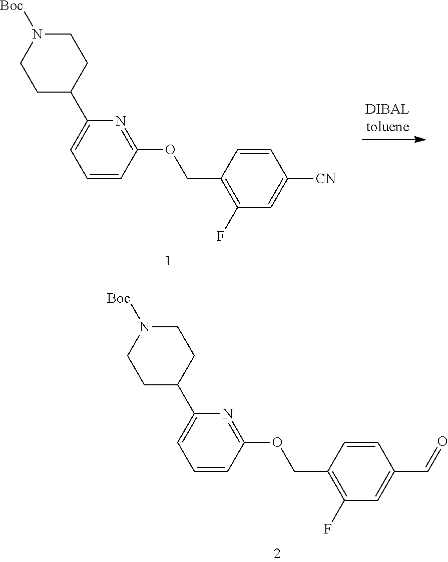 [SMILES: CC(C)(C)OC(=O)N1CCC(c2cccc(OCc3ccc(C#N)cc3F)n2)CC1 ; CC(C)(C)OC(=O)N1CCC(c2cccc(OCc3ccc(C=O)cc3F)n2)CC1]

A solution of tert-butyl 4-(6-((4-cyano-2-fluorobenzyl)oxy)pyridin-2-yl)piperidine-1-carboxylate 1 (3 g, 7.29 mmol, 1 eq) in toluene (30 mL) was degassed and purged with N₂ for 3 times, and then DIBAL (1 M, 8.75 mL, 1.2 eq) was added at 0° C. The mixture was stirred at 25° C. for 2 h under N₂ atmosphere. TLC indicated starting material was consumed completely. The combined reaction mixture was diluted with water (10 mL) and extracted with ethyl acetate (50 mL*2). The combined organic phases were concentrated in vacuo to give a residue, which was purified by column chromatography (SiO₂, Petroleum ether/Ethyl acetate=100/1 to 0/1) to provide product as a white solid (1.5 g, 3.62 mmol, 49.6% yield).

### Preparation of Intermediate (3), tert-Butyl-4-(6-((2-fluoro-4-((methoxyimino)methyl)benzyl)oxy)pyridin-2-yl)piperidine-1-carboxylate

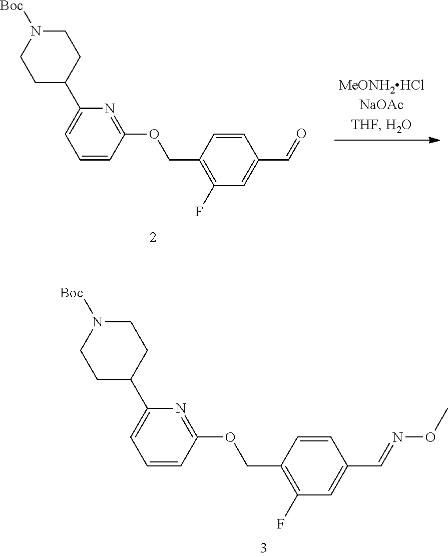 [SMILES: CC(C)(C)OC(=O)N1CCC(c2cccc(OCc3ccc(C=O)cc3F)n2)CC1 ; CO/N=C/c1ccc(COc2cccc(C3CCN(C(=O)OC(C)(C)C)CC3)n2)c(F)c1]

A mixture of tert-butyl 4-(6-((2-fluoro-4-formylbenzyl)oxy)pyridin-2-yl)piperidine-1-carboxylate 2 (1.5 g, 3.62 mmol, 1 eq), MeONH₂·HCl (605 mg, 7.24 mmol, 2 eq) and NaOAc (594 mg, 7.24 mmol, 2 eq) in THF (5 mL) and H₂O (15 mL) was degassed and purged with N₂ for 3 times, and then the mixture was stirred at 25° C. for 2 h under N₂ atmosphere. TLC indicated starting material was consumed completely. The reaction mixture was diluted with water (10 mL) and extracted with ethyl acetate (50 mL*2). The combined organic phases were concentrated in vacuo to give a residue, and the residue was purified by column chromatography (SiO₂, Petroleum ether/Ethyl acetate=100/1 to 0/1) to provide product as a white solid (770 mg, 1.74 mmol, 48% yield).

### Preparation of Intermediate (4) 3-fluoro-4-(((6-(piperidin-4-yl)pyridin-2-yl)oxy)methyl) benzaldehyde O-methyl oxime

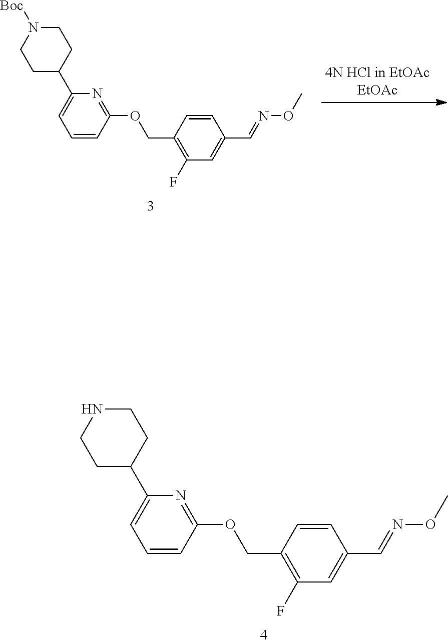 [SMILES: CO/N=C/c1ccc(COc2cccc(C3CCN(C(=O)OC(C)(C)C)CC3)n2)c(F)c1 ; CO/N=C/c1ccc(COc2cccc(C3CCNCC3)n2)c(F)c1]

To a solution of 3 (120 mg, 271 μmol, 1 eq) in EtOAc (1 mL) was added HCl/EtOAc (4 M, 2.00 mL, 29.6 eq). The mixture was stirred at 20° C. for 1 h. LC-MS showed starting material was consumed completely and desired mass was detected as main peak. The reaction mixture was concentrated under reduced pressure to give product as a white solid (100 mg, 263 μmol, 97% yield, HCl salt).

LCMS: Rt=0.427 min, MS cal.: 343.2, [M+H]⁺=344.2

¹H NMR (400 MHz, DMSO-d₆) δ=9.28-8.81 (br, 2H), 7.79-7.65 (m, 2H), 7.60-7.43 (m, 2H), 6.94 (d, J=7.2 Hz, 1H), 6.79 (d, J=8.4 Hz, 1H), 5.47 (s, 2H), 4.15-3.79 (s, 3H), 3.39 (m, 2H), 3.15-2.88 (m, 3H), 2.12-1.89 (m, 4H)

### Preparation of Intermediate (5) Methyl (S)-2-((4-(6-((2-Fluoro-4-((methoxyimino)methyl)benzyl)oxy)pyridin-2-yl)piperidin-1-yl)methyl)-4-methoxy-1-(oxetan-2-ylmethyl)-1H-benzo[d]imidazole-6-carboxylate

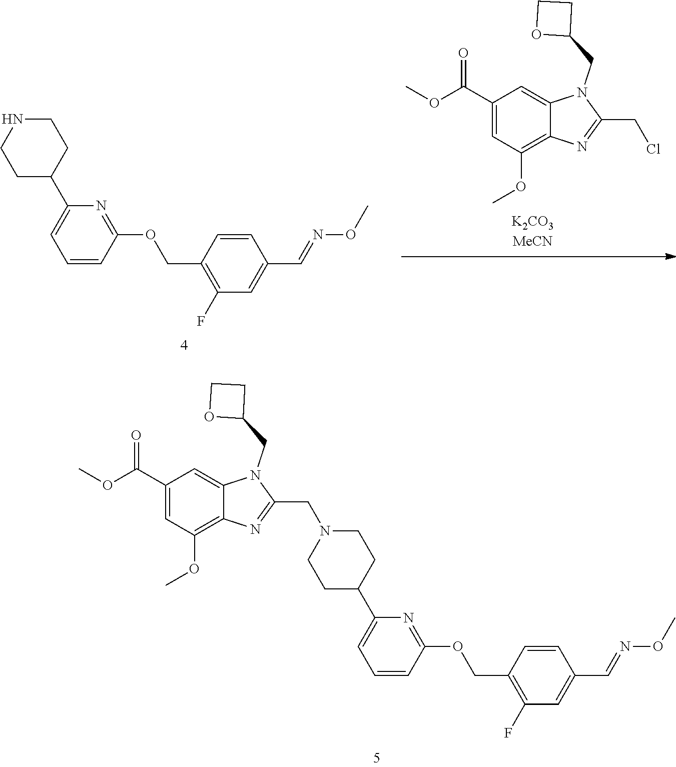

To a solution of 3-fluoro-4-(((6-(piperidin-4-yl)pyridin-2-yl)oxy)methyl) benzaldehyde O-methyl oxime 4 (100 mg, 291 μmol, 1 eq) in ACN (2 mL) was added methyl (S)-2-(chloromethyl)-4-methoxy-1-(oxetan-2-ylmethyl)-1H-benzo[d]imidazole-6-carboxylate (94.57 mg, 291.21 μmol, 1 eq) and K₂CO₃ (120.74 mg, 873.63 μmol, 3 eq). The mixture was stirred at 60° C. for 2 h. LC-MS showed starting material was consumed completely and desired mass was detected. The reaction mixture was diluted with H₂O (30 mL) and extracted with ethyl acetate (30 mL*3). The combined organic layers were washed with brine (10 mL), dried over Na₂SO₄, filtered, and concentrated under reduced pressure to give a residue. The residue was purified by column chromatography (SiO₂, EtOAc/MeOH=100/1 to 10/1) to provide product as a white solid (100 mg, 158 μmol, 54% yield).

LCMS: Rt=0.470 min, MS cal.: 631.3, [M+H]⁺=632.3

¹H NMR (400 MHz, DMSO-d₆) δ=8.23 (s, 1H), 7.94 (s, 1H), 7.70-7.52 (m, 2H), 7.44 (d, J=9.2 Hz, 2H), 7.27 (s, 1H), 6.88 (d, J=7.2 Hz, 1H), 6.69 (d, J=8.0 Hz, 1H), 5.41 (s, 2H), 5.15-5.06 (m, 1H), 4.85-4.74 (m, 1H), 4.66 (m, 1H), 4.52-4.43 (m, 1H), 4.35 (m, 1H), 4.01-3.85 (m, 10H), 3.77 (d, J=13.2 Hz, 1H), 2.99 (m, 1H), 2.84 (m, 1H), 2.75-2.66 (m, 1H), 2.64-2.56 (m, 1H), 2.48-2.38 (m, 1H), 2.28-2.12 (m, 2H), 1.84-1.62 (m, 4H)

### Preparation of (S)-2-((4-(6-((2-Fluoro-4-((methoxyimino)methyl)benzyl)oxy)pyridin-2-yl)piperidin-1-yl)methyl)-4-methoxy-1-(oxetan-2-ylmethyl)-1H-benzo[d]imidazole-6-carboxylic acid, Compound 9 (6)

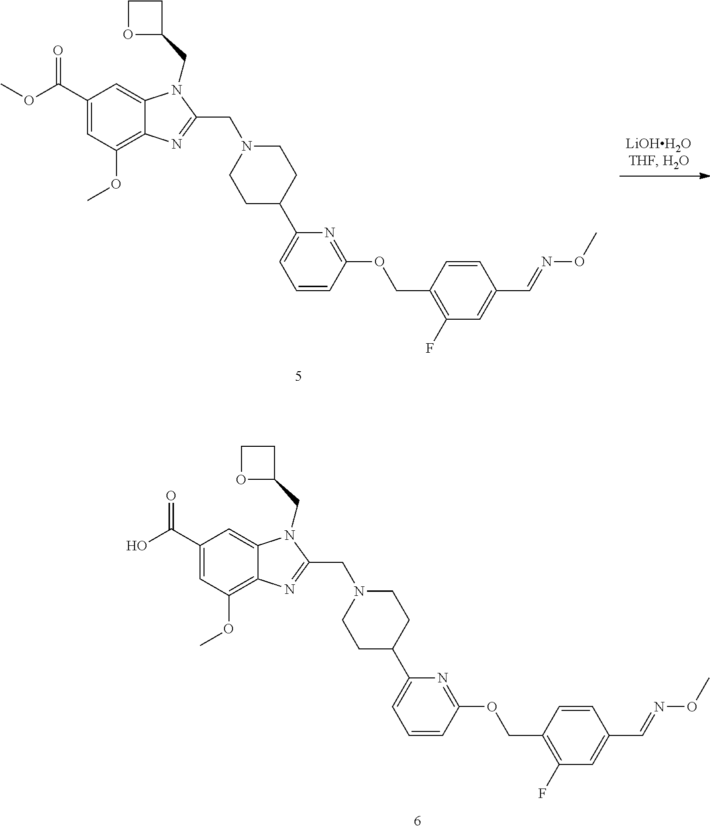

To a solution of methyl (S)-2-((4-(6-((2-Fluoro-4-((methoxyimino)methyl)benzyl)oxy)pyridin-2-yl)piperidin-1-yl)methyl)-4-methoxy-1-(oxetan-2-ylmethyl)-1H-benzo[d]imidazole-6-carboxylate, 5 (100 mg, 158 μmol, 1 eq) in THF (1.5 mL) was added LiOH·H₂O (10 mg, 237 μmol, 1.5 eq) in H₂O (0.5 mL). The mixture was stirred at 25° C. for 12 h. LC-MS showed starting material was consumed completely and desired mass was detected as main peak. The reaction mixture was filtered, and the filtrate was purified by prep-HPLC (neutral condition; Column: Waters Xbridge Prep OBD C18 150*40 mm*10 um; mobile phase: [H₂O (10 mM NH₄HCO₃)-ACN]; gradient: 20%-50% B over 8.0 min) to provide product as a white solid (28.3 mg, 45.8 μmol, 29% yield).

LCMS: Rt=1.521 min, MS cal.: 617.3, [M+H]⁺=618.4

HPLC: Rt=11.433 min, purity: 98.08%

¹H NMR (400 MHz, METHANOL-d₄) δ=8.07 (s, 1H), 7.95 (s, 1H), 7.61-7.57 (d, J=7.6 Hz, 1H), 7.53-7.51 (m, 1H), 7.47 (s, 1H), 7.40-7.35 (m, 2H), 6.84 (d, J=7.2 Hz, 1H), 6.67 (d, J=8.0 Hz, 1H), 5.47 (s, 2H), 5.29 (m, 1H), 4.88-4.86 (m, 1H), 4.76-4.70 (m, 1H), 4.69-4.61 (m, 1H), 4.52-4.45 (m, 1H), 4.14-3.98 (m, 5H), 3.94 (s, 3H), 3.14 (m, 1H), 3.05 (m, 1H), 2.88-2.77 (m, 1H), 2.75-2.65 (m, 1H), 2.62-2.51 (m, 1H), 2.50-2.37 (m, 2H), 1.99-1.84 (m, 4H)

### Example A10. —Preparation of Compound 57. 2-((4-((S)-2-(5-Chloropyridin-2-yl)-2-methylbenzo[d][1,3]dioxol-4-yl)piperidin-1-yl)methyl)-4-(1,1-difluoroethyl)-1-(((S)-oxetan-2-yl)methyl)-1H-benzo[d]imidazole-6-carboxylic acid (Compound 57) (10)

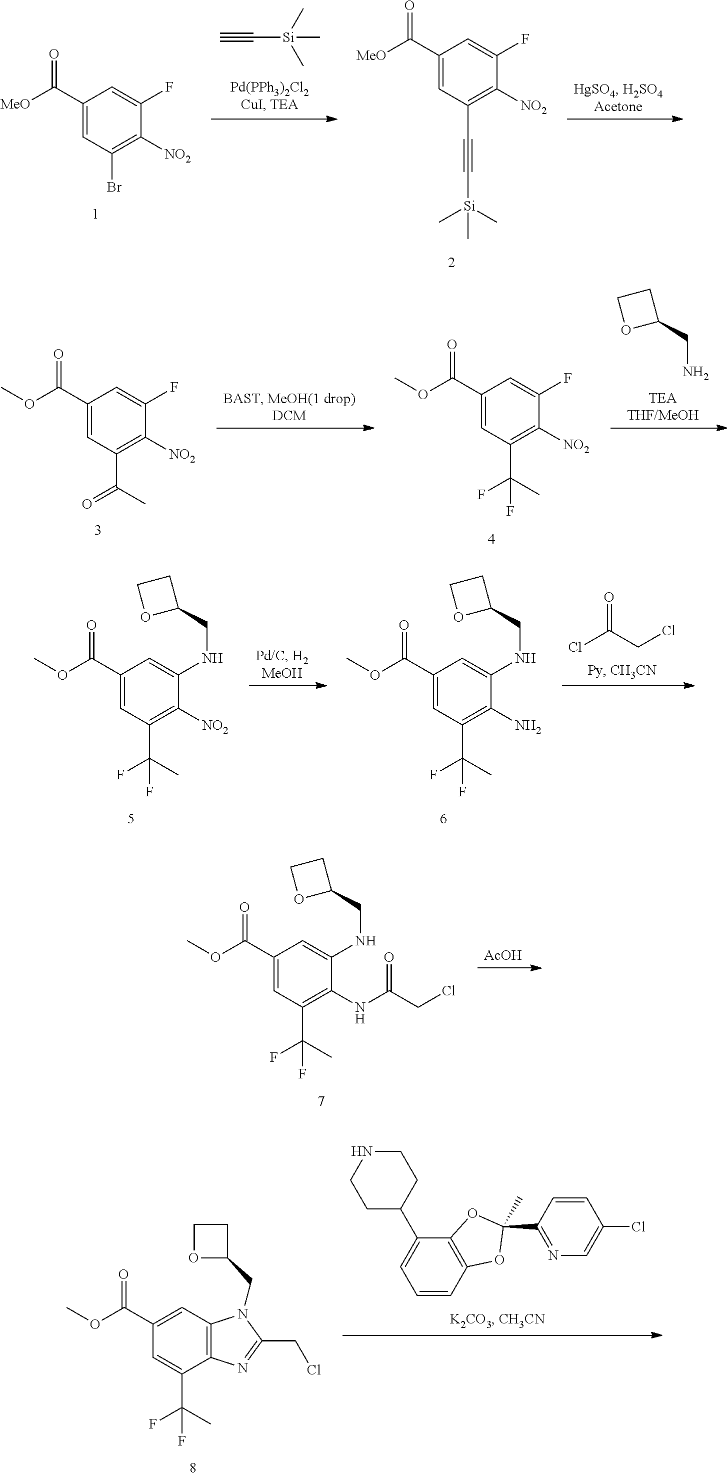

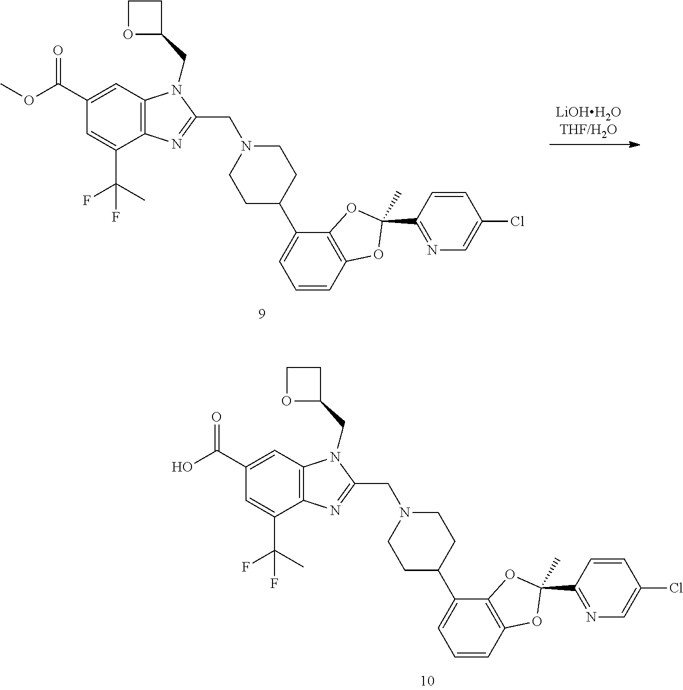 [SMILES: COC(=O)c1cc(C(C)(F)F)c2nc(CN3CCC(c4cccc5c4O[C@@](C)(c4ccc(Cl)cn4)O5)CC3)n(C[C@@H]3CCO3)c2c1 ; "LiOH·H2OTHF/H2O" ; CC(F)(F)c1cc(C(=O)O)cc2c1nc(CN1CCC(c3cccc4c3O[C@@](C)(c3ccc(Cl)cn3)O4)CC1)n2C[C@@H]1CCO1]

### Preparation of Intermediate (2) Methyl 3-fluoro-4-nitro-5-((trimethylsilyl)ethynyl)benzoate

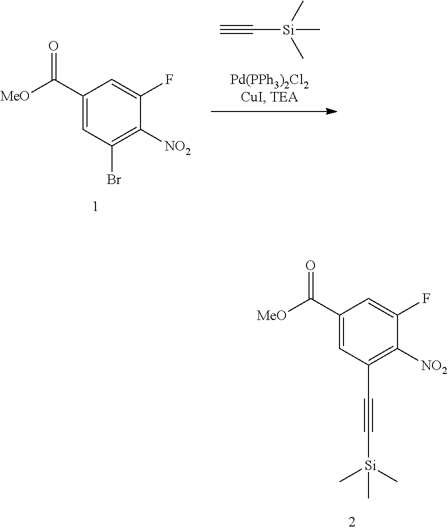

Methyl 3-bromo-5-fluoro-4-nitrobenzoate 1 (500 mg, 1.80 mmol, 1 eq) and Pd(PPh₃)₂Cl₂ (50.5 mg, 71.9 μmol, 0.04 eq) in TEA (10 mL) was degassed and purged with N₂ for 3 times. The mixture was stirred at 25° C. temperature for 10 min, and then ethynyltrimethylsilane (265 mg, 2.70 mmol, 374 μL, 1.5 eq) and CuI (68.5 mg, 360 μmol, 0.2 eq) in TEA (10 mL) was added at 25° C. The resulting mixture was stirred at 25° C. for 6 h. TLC (Petroleum ether/Ethyl acetate=5/1, R_{f}=0.4) indicated starting material was consumed completely and one new spot formed. The reaction mixture was diluted with water (100 mL) and extracted with EtOAc (70 mL*3). The organic phase was washed with brine (25 mL*1), dried over Na₂SO₄, filtered, and concentrated under reduced pressure to give a residue. The residue was purified by column chromatography (SiO₂, Petroleum ether/Ethyl acetate=20/1 to 0/1) to provide product (2.7 g, 9.14 mmol, 73% yield) as a yellow solid.

¹H NMR (400 MHz, CHLOROFORM-d) δ=8.04 (s, 1H), 7.85 (dd, J=1.2, 9.6 Hz, 1H), 3.98 (s, 3H), 0.26 (s, 9H)

### Preparation of Intermediate (3) Methyl 3-acetyl-5-fluoro-4-nitrobenzoate

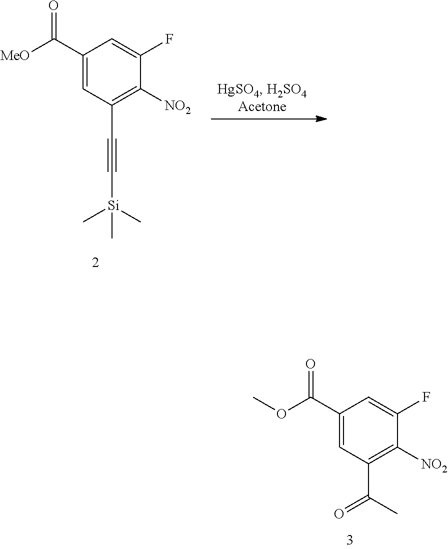 [SMILES: COC(=O)c1cc(F)c([N+](=O)[O-])c(C#C[Si](C)(C)C)c1 ; "HgSO4, H2SO4Acetone" ; COC(=O)c1cc(F)c([N+](=O)[O-])c(C(C)=O)c1]

Methyl 3-fluoro-4-nitro-5-((trimethylsilyl)ethynyl)benzoate 2 (4 g, 13.5 mmol, 1 eq), HgSO₄ (9.24 g, 31.2 mmol, 2.3 eq), and H₂SO₄ (611 mg, 6.23 mmol, 332 μL, 0.46 eq) in acetone (50 mL) were degassed and purged with N₂ for 3 times, and then the mixture was stirred at 60° C. for 12 h under a N₂ atmosphere. TLC (Petroleum ether/Ethyl acetate=1/1, R_{f}=0.3) indicated starting material was consumed completely and one new spot formed. The reaction mixture was diluted with water (100 mL) and extracted with EtOAc (100 mL*3). The organic phase was washed with brine (50 mL*2), dried over Na₂SO₄, filtered, and concentrated under reduced pressure to give a residue. The residue was purified by column chromatography (SiO₂, Petroleum ether/Ethyl acetate=9/1 to 0/1) to provide product (2.4 g, 9.95 mmol, 73% yield) as a brown solid.

LCMS: RT=1.622 min. MS cal.: 241.0; 242.0, [M−H]⁻=240.1; 240.9

¹H NMR (400 MHz, CHLOROFORM-d) δ=8.25 (t, J=1.2 Hz, 1H), 8.08 (dd, J=1.6, 8.8 Hz, 1H), 4.02 (s, 3H), 2.67 (s, 3H)

### Preparation of Intermediate (4) Methyl 3-(1,1-difluoroethyl)-5-fluoro-4-nitrobenzoate

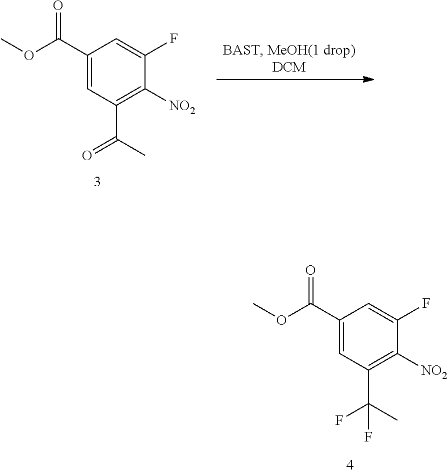 [SMILES: COC(=O)c1cc(F)c([N+](=O)[O-])c(C(C)=O)c1 ; "BAST, MeOH(1 drop)DCM" ; COC(=O)c1cc(F)c([N+](=O)[O-])c(C(C)(F)F)c1]

Methyl 3-acetyl-5-fluoro-4-nitrobenzoate 3 (500 mg, 2.07 mmol, 1 eq), BAST (4.13 g, 18.7 mmol, 4.09 mL, 9 eq) and MeOH (33.2 mg, 1.04 mmol, 42 μL, 0.5 eq) in DCM (25 mL) were degassed and purged with N₂ for 3 times, and then the mixture was stirred at 60° C. for 24 h under N₂ atmosphere. HPLC showed no starting material remained. Several new peaks were shown on HPLC and 77% of desired compound was detected. TLC (Petroleum ether/Ethyl acetate=2/1, R_{f}=0.4) indicated starting material was consumed completely and one new spot formed. At 0° C., the reaction solution was slowly added to water (20 mL). The mixture was extracted with DCM (30 mL*3). The organic phase was washed with brine (15 mL*2), dried over Na₂SO₄, filtered, and concentrated under reduced pressure to give a residue. The residue was purified by column chromatography (SiO₂, Petroleum ether/Ethyl acetate=20/1 to 0/1) to provide product (800 mg, 3.04 mmol, 73% yield) as a yellow solid.

HPLC: RT=3.092 min, purity=87%

¹H NMR (400 MHz, CHLOROFORM-d) δ=8.07 (s, 1H), 8.01 (d, J=8.8 Hz, 1H), 4.01 (s, 3H), 2.05 (t, J=18.4 Hz, 3H)

### Preparation of Intermediate (5) Methyl (S)-3-(1,1-difluoroethyl)-4-nitro-5-((oxetan-2-ylmethyl)amino)benzoate

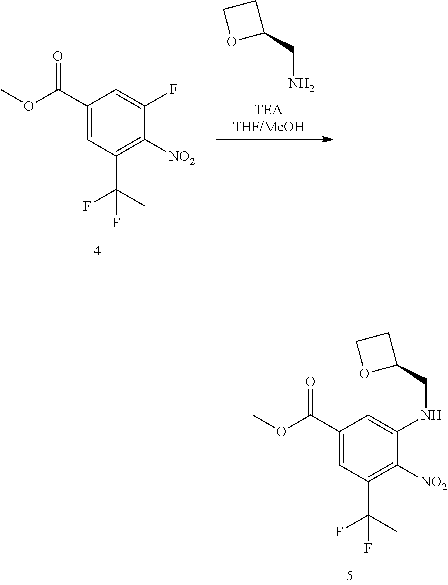

Methyl 3-(1,1-difluoroethyl)-5-fluoro-4-nitrobenzoate 4 (1 g, 3.80 mmol, 1 eq), (S)-oxetan-2-ylmethanamine (985 mg, 3.80 mmol, 1 eq, p-TSA), and TEA (385 mg, 3.80 mmol, 529 μL, 1 eq) in MeOH (5 mL) and THF (5 mL) was degassed and purged with N₂ for 3 times, and then the mixture was stirred at 60° C. for 12 h under a N₂ atmosphere. TLC (Petroleum ether/Ethyl acetate=1/1, R_{f}=0.2) indicated starting material was consumed completely and one new spot formed. The reaction mixture was diluted with water (10 mL) and extracted with DCM (10 mL*3). The organic phase was washed with brine (5 mL*2), dried over Na₂SO₄, filtered, and concentrated under reduced pressure to give a residue. The residue was purified by column chromatography (SiO₂, Petroleum ether/Ethyl acetate=9/1 to Petroleum ether/MeOH=0/1) to provide product (0.6 g, 1.82 mmol, 48% yield) as an orange oil.

LCMS: RT=0.508 min, MS cal.: 330.1; 331.1, [M+H]⁺=331.2; 332.3

¹H NMR (400 MHz, CHLOROFORM-d) δ=7.61 (s, 1H), 7.52 (s, 1H), 5.40 (br t, J=4.8 Hz, 1H), 5.15-4.98 (m, 1H), 4.79-4.68 (m, 1H), 4.63-4.52 (m, 1H), 3.96 (s, 3H), 3.58-3.45 (m, 2H), 2.81-2.66 (m, 1H), 2.63-2.50 (m, 1H), 2.19-2.00 (t, J=18.4 Hz, 3H)

### Preparation of Intermediate Methyl (S)-4-amino-3-(1,1-difluoroethyl)-5-((oxetan-2-ylmethyl)amino)benzoate (6)

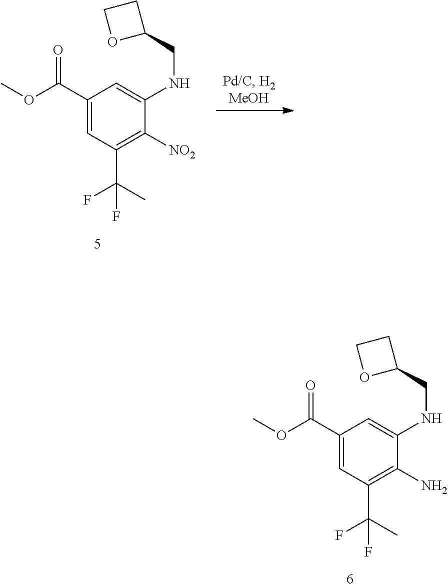 [SMILES: COC(=O)c1cc(NC[C@@H]2CCO2)c([N+](=O)[O-])c(C(C)(F)F)c1 ; "Pd/C, H2MeOH" ; COC(=O)c1cc(NC[C@@H]2CCO2)c(N)c(C(C)(F)F)c1]

Pd/C (275 mg, 10% purity) was added to methyl (S)-3-(1,1-difluoroethyl)-4-nitro-5-((oxetan-2-ylmethyl)amino)benzoate 5 (570 mg, 1.73 mmol, 1 eq) in MeOH (6 mL) under a N₂ atmosphere. The suspension was degassed and purged with H₂ for 3 times. The mixture was stirred under H₂ (15 psi) at 20° C. for 1 hr. LCMS showed starting material was consumed completely and one main peak with desired mass was detected. After 1 h, the reaction mixture was filtered, the cake was washed with MeOH (15 mL*3) and the filtrate was concentrated under reduced pressure to provide product (518 mg, crude) as a brown solid.

¹H NMR (400 MHz, CHLOROFORM-d) δ=7.68 (s, 1H), 7.47 (s, 1H), 5.12-5.11 (m, 1H), 4.77-4.75 (m, 1H), 4.68-4.54 (m, 1H), 4.57 (br s, 2H), 3.88 (s, 3H), 3.55-3.39 (m, 2H), 3.38-3.30 (m, 1H), 2.77-2.75 (m, 1H), 2.61-2.59 (m, 1H), 2.02 (t, J=18.8 Hz, 3H)

### Preparation of Intermediate Methyl (S)-4-(2-chloroacetamido)-3-(1,1-difluoroethyl)-5-((oxetan-2-ylmethyl)amino)benzoate (7)

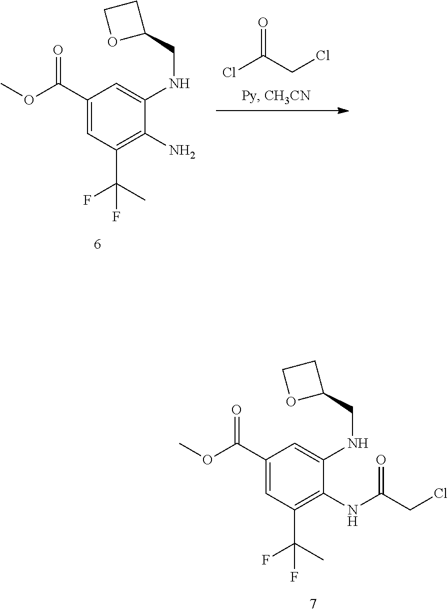

Methyl (S)-4-amino-3-(1,1-difluoroethyl)-5-((oxetan-2-ylmethyl)amino)benzoate 6 (500 mg, 1.66 mmol, 1 eq), pyridine (1.32 g, 16.7 mmol, 1.34 mL, 10 eq) and 2-chloroacetyl chloride (226 mg, 2.00 mmol, 159 μL, 1.2 eq) in CH₃CN (5 mL) were degassed and purged with N₂ for 3 times, and then the mixture was stirred at 20° C. for 1 h under N₂ atmosphere. TLC (Petroleum ether/Ethyl acetate=1/1, R_{f}=0.3) indicated starting material was consumed completely and one new spot formed. The reaction mixture was diluted with water (10 mL) and extracted with DCM (10 mL*3). The organic phase was dried over Na₂SO₄, filtered, and concentrated under reduced pressure to give a residue. The residue was purified by column chromatography (SiO₂, Petroleum ether/Ethyl acetate=5/1 to 0/1) to provide product (300 mg, 796 μmol, 48% yield) as a yellow solid.

LCMS: RT=1.537 min, MS cal.: 376.1; 378.1, [M+H]⁺=377.1; 379.1

### Preparation of Intermediate Methyl (S)-2-(chloromethyl)-4-(1,1-difluoroethyl)-1-(oxetan-2-ylmethyl)-1H-benzo[d]imidazole-6-carboxylate (8)

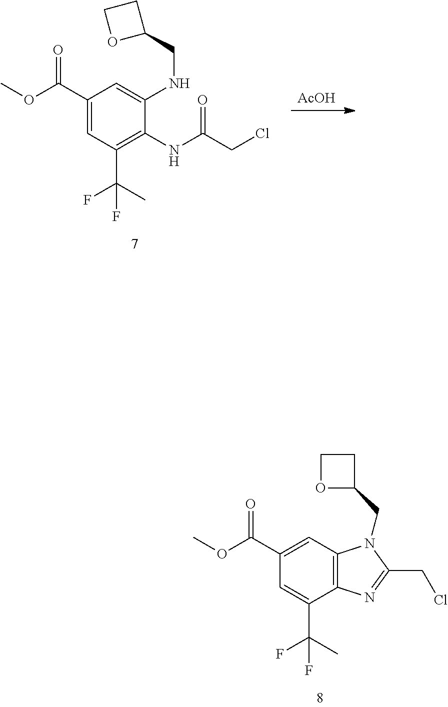 [SMILES: "AcOH" ; COC(=O)c1cc(NC[C@@H]2CCO2)c(NC(=O)CCl)c(C(C)(F)F)c1 ; COC(=O)c1cc(C(C)(F)F)c2nc(CCl)n(C[C@@H]3CCO3)c2c1]

Methyl (S)-4-(2-chloroacetamido)-3-(1,1-difluoroethyl)-5-((oxetan-2-ylmethyl)amino)benzoate 7 (300 mg, 796 μmol, 1 eq) in AcOH (1.5 mL) was degassed and purged with N₂ for 3 times, and then the mixture was stirred at 60° C. for 12 h under N₂ atmosphere. LCMS showed starting material was consumed completely and one main peak with desired mass was detected. TLC (Petroleum ether/Ethyl acetate=1/1, R_{f}=0.2) indicated starting material was consumed completely and one new spot formed. The reaction mixture was concentrated under reduced pressure to remove AcOH. The residue was diluted with water (5 mL) and extracted with DCM (5 mL*3). The organic phase was dried over Na₂SO₄, filtered, and concentrated under reduced pressure to give a residue. The residue was purified by column chromatography (SiO₂, Petroleum ether/Ethyl acetate=70/30 to Ethyl acetate/Methanol=0/1) to provide product (230 mg, 641 μmol, 81% yield) as a white solid.

LCMS: RT=1.737 min, MS cal.: 358.1; 360.1, [M+H]⁺=359.1

¹H NMR (400 MHz, CHLOROFORM-d) δ=8.21 (s, 2H), 5.31-5.21 (m, 1H), 5.08 (s, 2H), 4.72-4.61 (m, 2H), 4.60-4.52 (m, 1H), 4.40-4.38 (m, 1H), 3.98 (s, 3H), 2.79 (dtd, J=6.0, 8.0, 11.6 Hz, 1H), 2.50-2.39 (m, 1H), 2.32-2.20 (m, 3H)

### Preparation of Intermediate (9) Methyl 2-((4-((S)-2-(5-chloropyridin-2-yl)-2-methylbenzo[d][1,3]dioxol-4-yl)piperidin-1-yl)methyl)-4-(1,1-difluoroethyl)-1-(((S)-oxetan-2-yl)methyl)-1H-benzo[d]imidazole-6-carboxylate

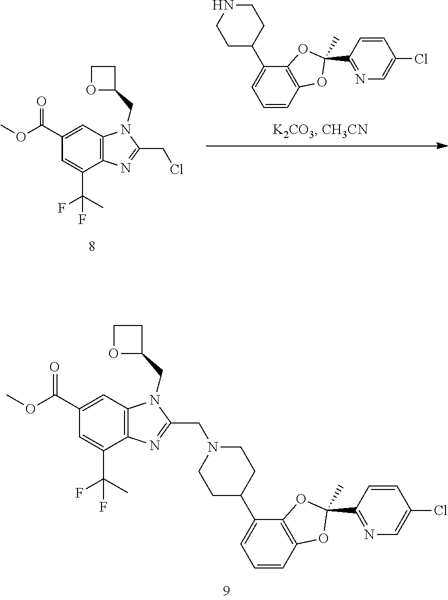 [SMILES: COC(=O)c1cc(C(C)(F)F)c2nc(CCl)n(C[C@@H]3CCO3)c2c1 ; C[C@]1(c2ccc(Cl)cn2)Oc2cccc(C3CCNCC3)c2O1 ; COC(=O)c1cc(C(C)(F)F)c2nc(CN3CCC(c4cccc5c4O[C@@](C)(c4ccc(Cl)cn4)O5)CC3)n(C[C@@H]3CCO3)c2c1]

Methyl (S)-2-(chloromethyl)-4-(1,1-difluoroethyl)-1-(oxetan-2-ylmethyl)-1H-benzo[d]imidazole-6-carboxylate 8 (120 mg, 334 μmol, 1 eq), (S)-5-chloro-2-(2-methyl-4-(piperidin-4-yl)benzo[d][1,3]dioxol-2-yl)pyridine (122 mg, 368 μmol, 1.1 eq), K₂CO₃ (416 mg, 3.01 mmol, 9 eq) in CH₃CN (1 mL) were degassed and purged with N₂ for 3 times, and then the mixture was stirred at 60° C. for 2 h under N₂ atmosphere. LCMS showed starting material was consumed completely and one main peak with desired mass was detected. TLC (Petroleum ether/Ethyl acetate=0/1, R_{f}=0.2) indicated starting material was consumed completely and one new spot formed. The reaction mixture was diluted with water (5 mL) and extracted with DCM (5 mL*3). The organic phase was dried over Na₂SO₄, filtered, and concentrated under reduced pressure to give a residue. The residue was purified by column chromatography (SiO₂, Petroleum ether/Ethyl acetate=7/3 to Ethyl acetate/Methanol=0/1) to provide product (130 mg, 199 μmol, 60% yield) as a white solid.

LCMS: RT=2.393 min, MS cal.: 652.2; 653.2, [M+H]⁺=653.2

### Preparation of 2-((4-((S)-2-(5-Chloropyridin-2-yl)-2-methylbenzo[d][1,3]dioxol-4-yl)piperidin-1-yl)methyl)-4-(1,1-difluoroethyl)-1-(((S)-oxetan-2-yl)methyl)-1H-benzo[d]imidazole-6-carboxylic acid, Compound 57 (10)

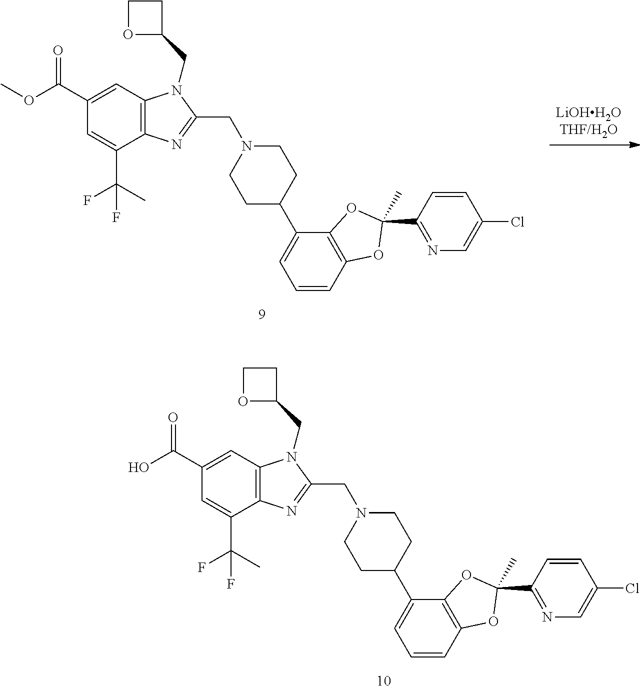 [SMILES: COC(=O)c1cc(C(C)(F)F)c2nc(CN3CCC(c4cccc5c4O[C@@](C)(c4ccc(Cl)cn4)O5)CC3)n(C[C@@H]3CCO3)c2c1 ; "LiOH·H2OTHF/H2O" ; CC(F)(F)c1cc(C(=O)O)cc2c1nc(CN1CCC(c3cccc4c3O[C@@](C)(c3ccc(Cl)cn3)O4)CC1)n2C[C@@H]1CCO1]

Methyl 2-((4-((S)-2-(5-chloropyridin-2-yl)-2-methylbenzo[d][1,3]dioxol-4-yl)piperidin-1-yl)methyl)-4-(1,1-difluoroethyl)-1-(((S)-oxetan-2-yl)methyl)-1H-benzo[d]imidazole-6-carboxylate 9 (0.1 g, 153 μmol, 1 eq), and LiOH·H₂O (9.64 mg, 230 μmol, 1.5 eq) in THF (0.7 mL) and H₂O (0.3 mL) were degassed and purged with N₂ for 3 times, and then the mixture was stirred at 25° C. for 12 h under N₂ atmosphere. LC-MS showed starting material was consumed completely and one main peak with desired mass was detected. The reaction mixture was filtered. The filtrate was purified by prep-HPLC (column: Waters Xbridge Prep OBD C18 150*40 mm*10 um; mobile phase: [H₂O (10 mM NH₄HCO₃)-ACN]; gradient: 30%-60% B over 8.0 min) to provide product (29 mg, 44.7 μmol, 29% yield) as a white solid.

LCMS: RT=2.742 min, MS cal.: 638.2; 639.2, [M+H]⁺=639.2

HPLC: RT=11.832 min, purity: 100.00%

SFC: RT=4.923 min, purity: 100.00%

¹H NMR (400 MHz, METHANOL-d₄) δ=8.61 (d, J=2.0 Hz, 1H), 8.41 (s, 1H), 8.12 (s, 1H), 7.87 (dd, J=2.4, 8.4 Hz, 1H), 7.65 (d, J=8.4 Hz, 1H), 6.83-6.76 (m, 1H), 6.75-6.70 (m, 2H), 5.27-5.25 (m, 1H), 4.93-4.87 (m, 1H), 4.73 (dd, J=2.4, 15.6 Hz, 1H), 4.66-4.58 (m, 1H), 4.50-4.48 (m, 1H), 4.21-4.15 (d, J=14.0 Hz, 1H), 4.10-4.04 (d, J=14.0 Hz, 1H), 3.23-3.21 (m, 1H), 3.0-3.07 (m, 1H), 2.87-2.70 (m, 2H), 2.61-2.39 (m, 3H), 2.18 (t, J=18.8 Hz, 3H), 2.02 (s, 3H), 2.00-1.82 (m, 4H)

### Example A11. —Preparation of Compound 71. 2-((4-((R)-2-(5-Chloropyridin-2-yl)-2-methylbenzo[d][1,3]dioxol-4-yl)piperidin-1-yl)methyl)-4-(1,1-difluoroethyl)-1-(((S)-oxetan-2-yl)methyl)-1H-benzo[d]imidazole-6-carboxylic acid (Compound 71) (3)

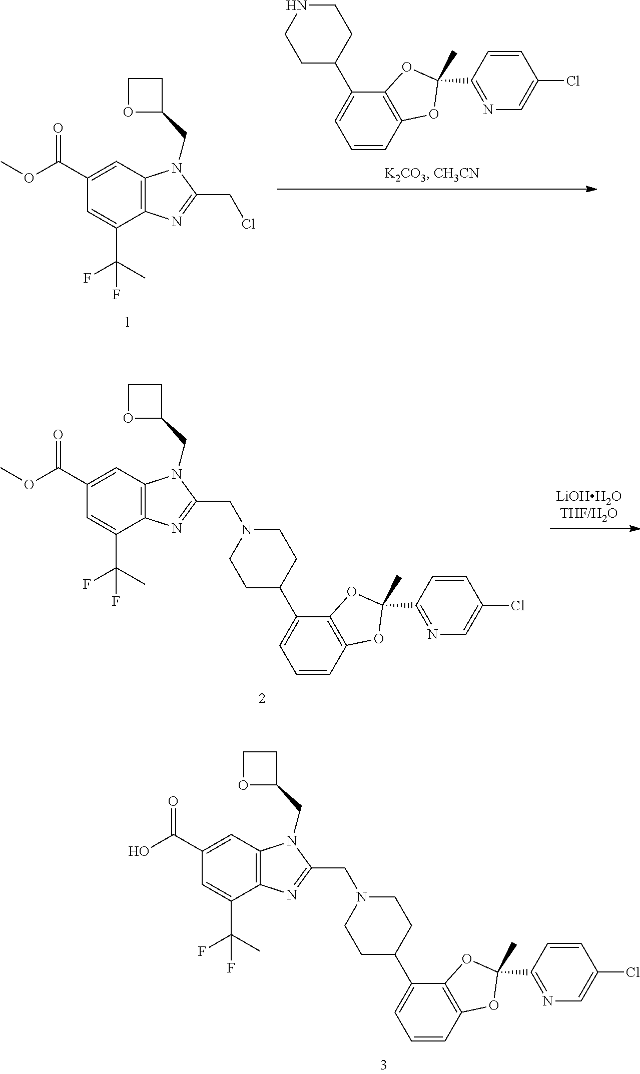 [SMILES: COC(=O)c1cc(C(C)(F)F)c2nc(CCl)n(C[C@@H]3CCO3)c2c1 ; C[C@@]1(c2ccc(Cl)cn2)Oc2cccc(C3CCNCC3)c2O1 ; COC(=O)c1cc(C(C)(F)F)c2nc(CN3CCC(c4cccc5c4O[C@](C)(c4ccc(Cl)cn4)O5)CC3)n(C[C@@H]3CCO3)c2c1 ; "LiOH·H2OTHF/H2O" ; CC(F)(F)c1cc(C(=O)O)cc2c1nc(CN1CCC(c3cccc4c3O[C@](C)(c3ccc(Cl)cn3)O4)CC1)n2C[C@@H]1CCO1]

### Preparation of Intermediate (2) Methyl 2-((4-((R)-2-(5-chloropyridin-2-yl)-2-methylbenzo[d][1,3]dioxol-4-yl)piperidin-1-yl)methyl)-4-(1,1-difluoroethyl)-1-(((S)-oxetan-2-yl)methyl)-1H-benzo[d]imidazole-6-carboxylate

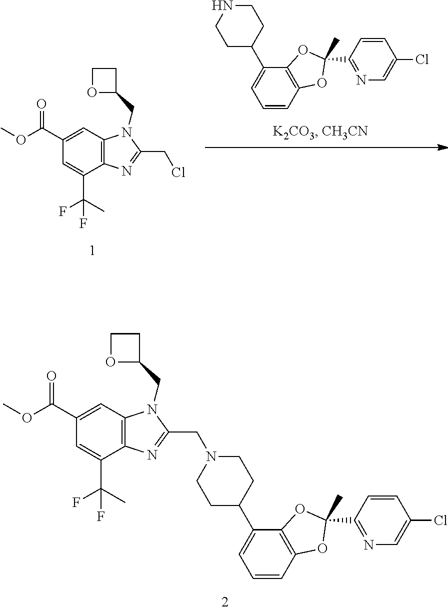 [SMILES: COC(=O)c1cc(C(C)(F)F)c2nc(CCl)n(C[C@@H]3CCO3)c2c1 ; C[C@@]1(c2ccc(Cl)cn2)Oc2cccc(C3CCNCC3)c2O1 ; COC(=O)c1cc(C(C)(F)F)c2nc(CN3CCC(c4cccc5c4O[C@](C)(c4ccc(Cl)cn4)O5)CC3)n(C[C@@H]3CCO3)c2c1]

Methyl (S)-2-(chloromethyl)-4-(1,1-difluoroethyl)-1-(oxetan-2-ylmethyl)-1H-benzo[d]imidazole-6-carboxylate 1 (120 mg, 334 μmol, 1 eq), (R)-5-chloro-2-(2-methyl-4-(piperidin-4-yl)benzo[d][1,3]dioxol-2-yl)pyridine (122 mg, 368 μmol, 1.1 eq) and K₂CO₃ (139 mg, 1.00 mmol, 3 eq) in CH₃CN (3 mL) was degassed and purged with N₂ for 3 times, and then the mixture was stirred at 60° C. for 2 h under N₂ atmosphere. LCMS showed the starting material was consumed completely. The reaction mixture was diluted with water (5 mL) and extracted with ethyl acetate (5 mL*4). The combined organic phase was washed with brine (8 mL), dried with anhydrous Na₂SO₄, filtered, and concentrated in vacuum. The residue was purified by column chromatography (SiO₂, Petroleum ether/Ethyl acetate=100/1 to 0/1) to provide product (170 mg, crude) as a white solid.

LCMS: RT=2.421 min, MS cal.: 652.2; 653.2, [M+H]⁺=653.2

### Preparation of 2-((4-((R)-2-(5-Chloropyridin-2-yl)-2-methylbenzo[d][1,3]dioxol-4-yl)piperidin-1-yl)methyl)-4-(1,1-difluoroethyl)-1-(((S)-oxetan-2-yl)methyl)-1H-benzo[d]imidazole-6-carboxylic acid, Compound 71 (3)

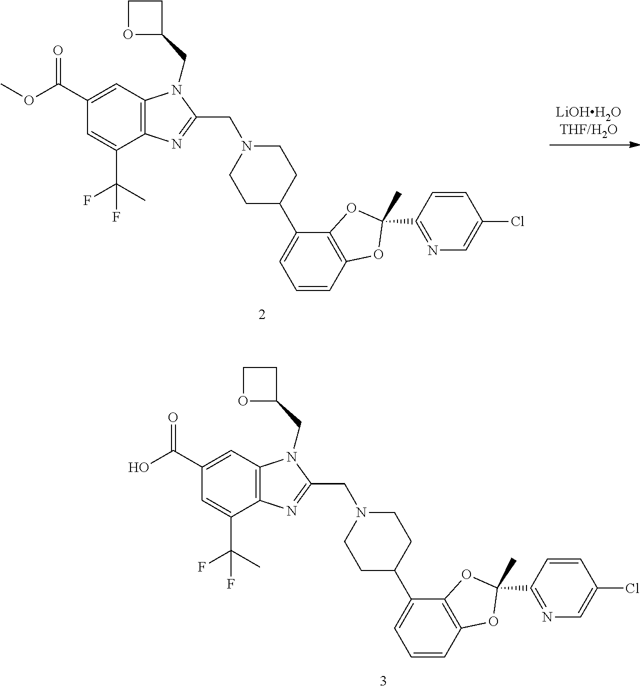 [SMILES: COC(=O)c1cc(C(C)(F)F)c2nc(CN3CCC(c4cccc5c4O[C@](C)(c4ccc(Cl)cn4)O5)CC3)n(C[C@@H]3CCO3)c2c1 ; "LiOH·H2OTHF/H2O" ; CC(F)(F)c1cc(C(=O)O)cc2c1nc(CN1CCC(c3cccc4c3O[C@](C)(c3ccc(Cl)cn3)O4)CC1)n2C[C@@H]1CCO1]

Methyl 2-((4-((R)-2-(5-chloropyridin-2-yl)-2-methylbenzo[d][1,3]dioxol-4-yl)piperidin-1-yl)methyl)-4-(1,1-difluoroethyl)-1-(((S)-oxetan-2-yl)methyl)-1H-benzo[d]imidazole-6-carboxylate 2 (150 mg, 230 μmol, 1 eq) and LiOH·H₂O (14.5 mg, 345 μmol, 1.5 eq), in THF (1.4 mL) and H₂O (0.6 mL) were degassed and purged with N₂ for 3 times, and then the mixture was stirred at 25° C. for 12 h under N₂ atmosphere. LCMS showed the starting material was consumed completely. The reaction mixture was concentrated under reduced pressure to remove solvent. The residue was purified by prep-HPLC (column: Waters Xbridge Prep OBD C18 150*40 mm*10 um; mobile phase: [H₂O (10 mM NH₄HCO₃)—CH₃CN]; gradient: 20%-50% B over 8.0 min) to provide product (31 mg, 48.6 μmol, 21% yield) as a white solid.

LCMS: RT=2.770 min, MS cal.: 638.2, [M+H]⁺=639.0

HPLC: RT=11.795 min, purity: 98.35%

SFC: RT=5.021 min, purity: 100.00%

¹H NMR (400 MHz, METHANOL-d4) δ=8.61 (s, 1H), 8.41 (s, 1H), 8.12 (s, 1H), 7.88 (dd, J=2.4, 8.4 Hz, 1H), 7.65 (d, J=8.4 Hz, 1H), 6.83-6.69 (m, 3H), 5.32-5.22 (m, 1H), 4.88-4.86 (m, 1H), 4.75-4.73 (m, 1H), 4.69-4.60 (m, 1H), 4.53-4.44 (m, 1H), 4.21 (d, J=14.0 Hz, 1H), 4.11 (d, J=14.0 Hz, 1H), 3.26-3.18 (m, 1H), 3.15-3.03 (m, 1H), 2.87-2.73 (m, 2H), 2.61-2.41 (m, 3H), 2.19 (t, J=18.4 Hz, 3H), 2.02 (s, 3H), 1.98-1.79 (m, 4H)

### Example A12. —Preparation of Compound 72. 2-((4-((S)-2-(5-Chloropyridin-2-yl)-2-methylbenzo[d][1,3]dioxol-4-yl)piperidin-1-yl)methyl)-1-(((S)-oxetan-2-yl)methyl)-4-(oxetan-3-yloxy)-1H-benzo[d]imidazole-6-carboxylic acid (Compound 72) (7)

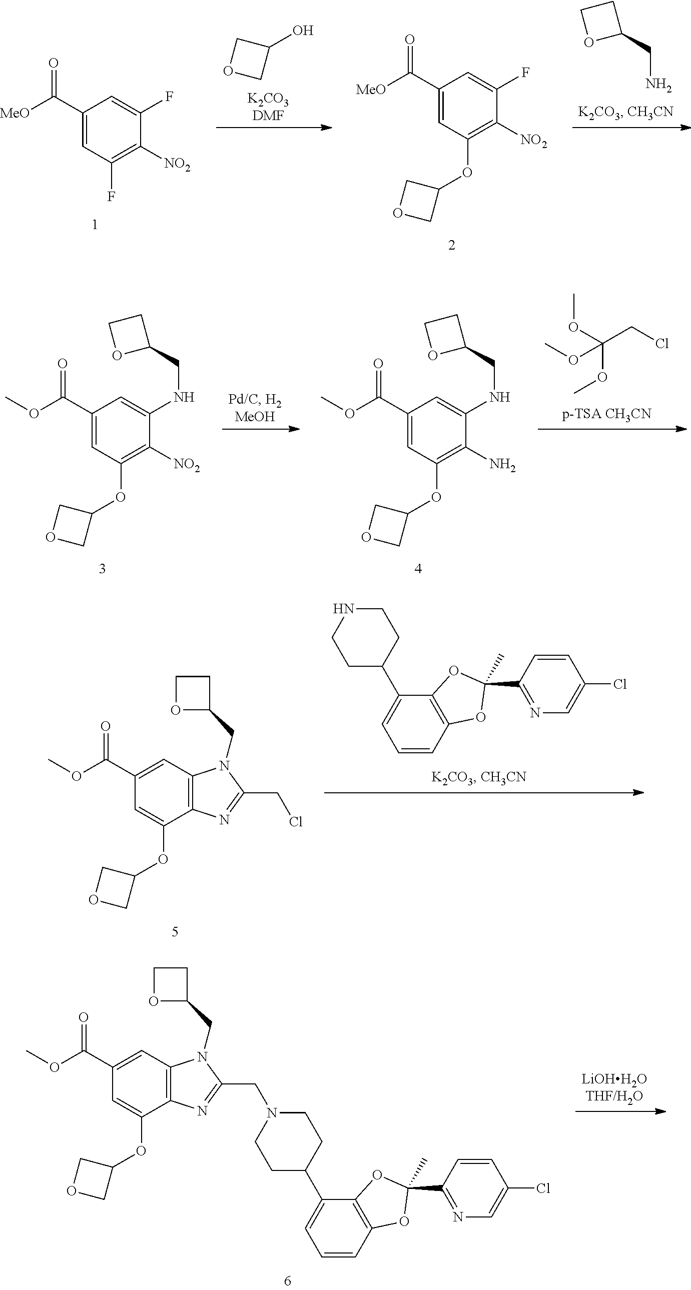

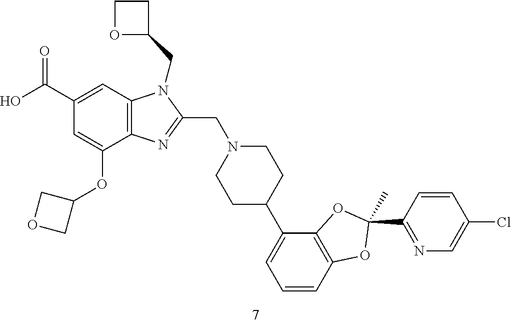 [SMILES: C[C@]1(c2ccc(Cl)cn2)Oc2cccc(C3CCN(Cc4nc5c(OC6COC6)cc(C(=O)O)cc5n4C[C@@H]4CCO4)CC3)c2O1]

### Preparation of Intermediate (2) Methyl 3-fluoro-4-nitro-5-(oxetan-3-yloxy)benzoate

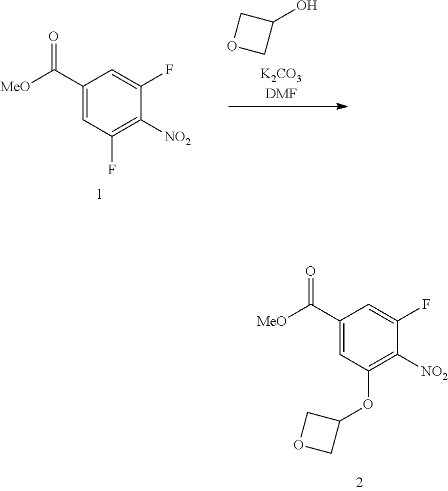 [SMILES: COC(=O)c1cc(F)c([N+](=O)[O-])c(F)c1 ; OC1COC1 ; COC(=O)c1cc(F)c([N+](=O)[O-])c(OC2COC2)c1]

Methyl 3,5-difluoro-4-nitrobenzoate 1 (1 g, 4.61 mmol, 1 eq), oxetan-3-ol (375 mg, 5.07 mmol, 1.1 eq) and K₂CO₃ (1.91 g, 13.82 mmol, 3 eq) in DMF (10 mL) was degassed and purged with N₂ for 3 times, and then the mixture was stirred at 60° C. for 5 h under N₂ atmosphere. TLC (Petroleum ether/Ethyl acetate=1/1, R_{f}=0.38) indicated starting material was consumed completely. The reaction mixture was diluted with H₂O (10 mL) and extracted with EtOAc (10 mL*3). The combined organic layers were washed with brine (10 mL*2), dried over Na₂SO₄, filtered, and concentrated under reduced pressure to give a residue. The residue was purified by column chromatography (SiO₂, Petroleum ether/Ethyl acetate=1/1) to provide product (750 mg, 2.77 mmol, 60% yield) as a white solid.

¹H NMR (400 MHz, CHLOROFORM-d) δ=7.56 (dd, J=1.2, 9.2 Hz, 1H), 7.09 (t, J=1.2 Hz, 1H), 5.42-5.35 (m, 1H), 5.03-4.99 (m, 2H), 4.80-4.77 (m, 2H), 3.97 (s, 3H)

### Preparation of Intermediate (3), Methyl (S)-4-nitro-3-((oxetan-2-ylmethyl)amino)-5-(oxetan-3-yloxy)benzoate

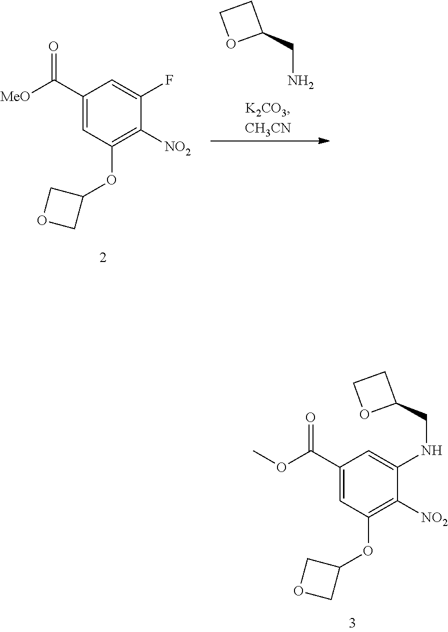 [SMILES: COC(=O)c1cc(F)c([N+](=O)[O-])c(OC2COC2)c1 ; NC[C@@H]1CCO1 ; COC(=O)c1cc(NC[C@@H]2CCO2)c([N+](=O)[O-])c(OC2COC2)c1]

(S)-Oxetan-2-ylmethanamine (1.08 g, 4.15 mmol, 1.5 eq, p-TSA), K₂CO₃ (1.15 g, 8.30 mmol, 3 eq) and methyl 3-fluoro-4-nitro-5-(oxetan-3-yloxy)benzoate 2 (750 mg, 2.77 mmol, 1 eq) in ACN (8 mL) were degassed and purged with N₂ for 3 times, and then the mixture was stirred at 60° C. for 12 h under N₂ atmosphere. LCMS showed starting material was consumed completely. The reaction mixture was diluted with H₂O (10 mL) and extracted with EtOAc (10 mL*3). The combined organic layers were washed with brine (10 mL*2), dried over Na₂SO₄, filtered, and concentrated under reduced pressure to give a residue. The residue was purified by column chromatography (SiO₂, Petroleum ether/Ethyl acetate=100/1 to 0/1) to provide product (450 mg, 1.33 mmol, 48% yield) as a white solid.

LCMS: RT=0.720 min, MS cal.: 338.3, [M+H]⁺=339.2

¹H NMR (400 MHz, CHLOROFORM-d) δ=7.18 (d, J=1.2 Hz, 1H), 6.48 (d, J=1.6 Hz, 1H), 6.25 (br t, J=5.2 Hz, 1H), 5.36-5.29 (m, 1H), 5.12-5.06 (m, 1H), 4.99-4.97 (m, 2H), 4.80-4.78 (m, 2H), 4.73-4.71 (m, 1H), 4.59-4.57 (m, 1H), 3.93 (s, 3H), 3.50 (t, J=5.2 Hz, 2H), 2.79-2.70 (m, 1H), 2.61-2.52 (m, 1H)

### Preparation of Intermediate (4) Methyl (S)-4-amino-3-((oxetan-2-ylmethyl)amino)-5-(oxetan-3-yloxy)benzoate

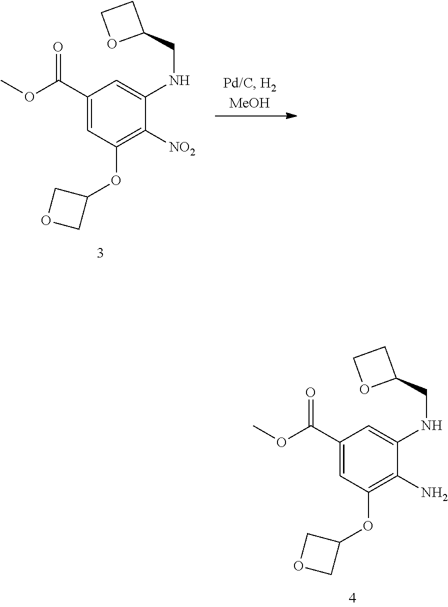 [SMILES: COC(=O)c1cc(NC[C@@H]2CCO2)c([N+](=O)[O-])c(OC2COC2)c1 ; COC(=O)c1cc(NC[C@@H]2CCO2)c(N)c(OC2COC2)c1]

To a solution of methyl (S)-4-nitro-3-((oxetan-2-ylmethyl)amino)-5-(oxetan-3-yloxy)benzoate 3 (450 mg, 1.33 mmol, 1 eq) in MeOH (8 mL) was added Pd/C (142 mg, 10%). The mixture was stirred at 20° C. for 1 h. LCMS showed starting material was consumed completely. The reaction mixture was filtered and the filter was concentrated to provide product (370 mg, 1.20 mmol, 90% yield) which was used into the next step without further purification.

LCMS: RT=0.622 min, MS cal.: 308.3, [M+H]⁺=309.2

¹H NMR (400 MHz, CHLOROFORM-d) δ=7.17 (d, J=1.2 Hz, 1H), 6.74 (d, J=1.2 Hz, 1H), 5.33-5.27 (m, 1H), 5.11-5.09 (m, 1H), 5.03-5.01 (m, 2H), 4.82-4.72 (m, 3H), 4.62-4.61 (m, 1H), 4.00 (br s, 2H), 3.87 (s, 3H), 3.61 (br s, 1H), 3.50-3.35 (m, 2H), 2.81-2.72 (m, 1H), 2.61-2.59 (m, 1H)

### Preparation of Intermediate (5) Methyl (S)-2-(chloromethyl)-1-(oxetan-2-ylmethyl)-4-(oxetan-3-yloxy)-1H-benzo[d]imidazole-6-carboxylate

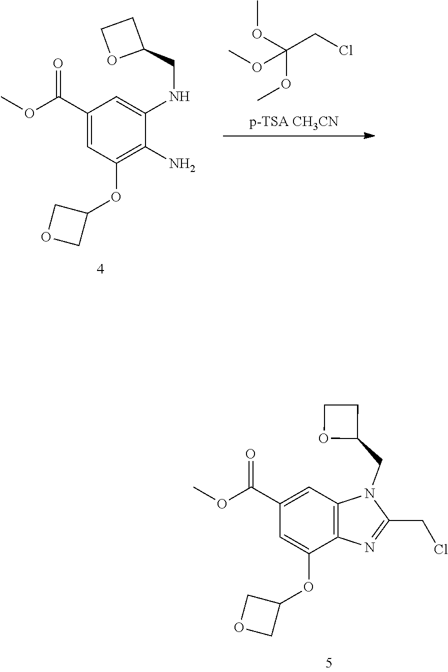

Methyl (S)-4-amino-3-((oxetan-2-ylmethyl)amino)-5-(oxetan-3-yloxy)benzoate 4 (300 mg, 973 μmol, 1 eq), 2-chloro-1,1,1-trimethoxyethane (451 mg, 2.92 mmol, 393 μL, 3 eq) and p-TSA (83.8 mg, 486 μmol, 0.5 eq) in ACN (5 mL) was degassed and purged with N₂ for 3 times, and then the mixture was stirred at 60° C. for 2 h under N₂ atmosphere. LCMS showed starting material was consumed completely. The reaction was concentrated under reduced pressure to give a residue. The residue was purified by column chromatography (SiO₂, Petroleum ether/Ethyl acetate=100/1 to 0/1) to provide product (140 mg, 382 μmol, 39% yield) as a white solid.

LCMS: RT=1.224 min, MS cal.: 366.8, [M+H]⁺=367.1

¹H NMR (400 MHz, CHLOROFORM-d) δ=7.79 (d, J=1.2 Hz, 1H), 7.03 (d, J=1.2 Hz, 1H), 5.56-5.51 (m, 1H), 5.24-5.17 (m, 1H), 5.10-5.02 (m, 4H), 4.98-4.96 (m, 2H), 4.68-4.49 (m, 3H), 4.38-4.24 (m, 1H), 3.94 (s, 3H), 2.77-2.72 (m, 1H), 2.42-2.37 (m, 1H)

### Preparation of Intermediate (6) Methyl 2-((4-((S)-2-(5-chloropyridin-2-yl)-2-methylbenzo[d][1,3] dioxol-4-yl)piperidin-1-yl)methyl)-1-(((S)-oxetan-2-yl)methyl)-4-(oxetan-3-yloxy)-1H-benzo[d]imidazole-6-carboxylate

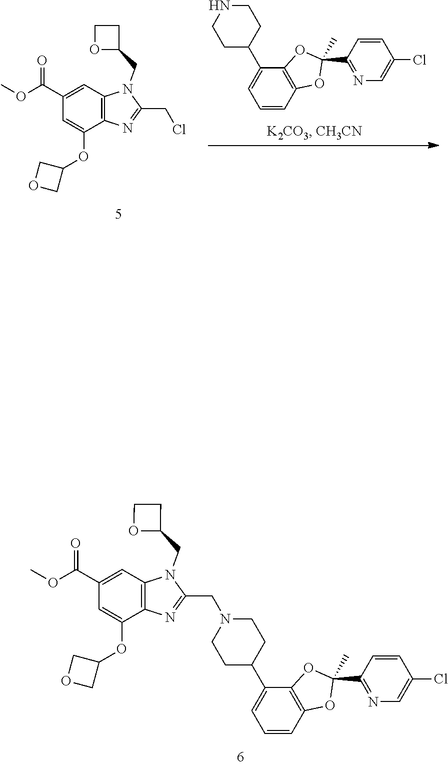 [SMILES: COC(=O)c1cc(OC2COC2)c2nc(CCl)n(C[C@@H]3CCO3)c2c1 ; C[C@]1(c2ccc(Cl)cn2)Oc2cccc(C3CCNCC3)c2O1 ; COC(=O)c1cc(OC2COC2)c2nc(CN3CCC(c4cccc5c4O[C@@](C)(c4ccc(Cl)cn4)O5)CC3)n(C[C@@H]3CCO3)c2c1]

Methyl (S)-2-(chloromethyl)-1-(oxetan-2-ylmethyl)-4-(oxetan-3-yloxy)-1H-benzo[d]imidazole-6-carboxylate 5 (80 mg, 218 μmol, 1 eq), (S)-5-chloro-2-(2-methyl-4-(piperidin-4-yl)benzo[d][1,3]dioxol-2-yl)pyridine (72.2 mg, 218 μmol, 1 eq) and K₂CO₃ (90.4 mg, 654 μmol, 3 eq) in ACN (2 mL) was degassed and purged with N₂ for 3 times, and then the mixture was stirred at 60° C. for 2 h under N₂ atmosphere. LCMS showed the starting material was consumed completely. The reaction mixture was diluted with H₂O (8 mL) and extracted with EtOAc (10 mL*3). The combined organic layers were washed with brine 10 mL, dried over Na₂SO₄, filtered, and concentrated under reduced pressure to give a residue. The residue was purified by column chromatography (SiO₂, Ethyl acetate/MeOH=100/1 to 1/1) to provide product (130 mg, 197 μmol, 90% yield) as a white solid.

LCMS: RT=2.100 min, MS cal.: 661.2, [M+H]⁺=661.2

### Preparation of 2-((4-((S)-2-(5-Chloropyridin-2-yl)-2-methylbenzo[d][1,3]dioxol-4-yl)piperidin-1-yl)methyl)-1-(((S)-oxetan-2-yl)methyl)-4-(oxetan-3-yloxy)-1H-benzo[d]imidazole-6-carboxylic acid, Compound 72 (7)

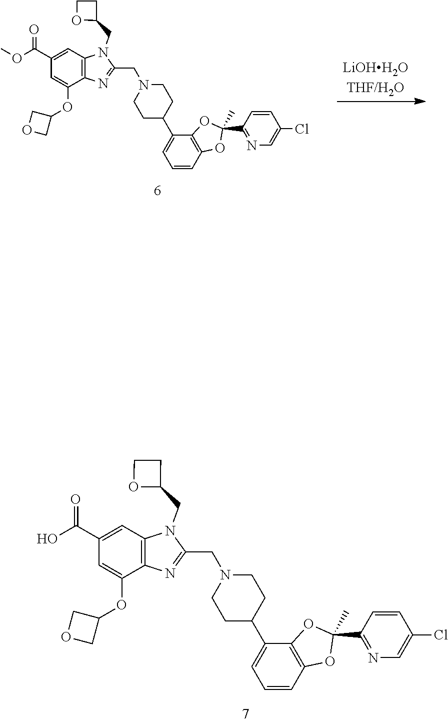 [SMILES: COC(=O)c1cc(OC2COC2)c2nc(CN3CCC(c4cccc5c4O[C@@](C)(c4ccc(Cl)cn4)O5)CC3)n(C[C@@H]3CCO3)c2c1 ; C[C@]1(c2ccc(Cl)cn2)Oc2cccc(C3CCN(Cc4nc5c(OC6COC6)cc(C(=O)O)cc5n4C[C@@H]4CCO4)CC3)c2O1]

Methyl 2-((4-((S)-2-(5-chloropyridin-2-yl)-2-methylbenzo[d][1,3]dioxol-4-yl)piperidin-1-yl)methyl)-1-(((S)-oxetan-2-yl)methyl)-4-(oxetan-3-yloxy)-1H-benzo[d]imidazole-6-carboxylate 6 (80 mg, 121 μmol, 1 eq) and LiOH·H₂O (7.62 mg, 182 μmol, 1.5 eq) in H₂O (0.3 mL) and THF (0.7 mL) were degassed and purged with N₂ for 3 times, and then the mixture was stirred at 25° C. for 12 hr under N₂ atmosphere. LCMS showed the starting material was consumed completely. The crude product was purified by reversed-phase HPLC (column: Waters Xbridge Prep OBD C18 150*40 mm*10 um; mobile phase: [H₂O (10 mM NH₄HCO₃)-ACN]; gradient: 20%-80% B over 8.0 min) to provide product (26.4 mg, 40.8 μmol, 34% yield) as a white solid.

LCMS: RT=2.613 min, MS cal.: 647.1, [M+H]⁺=647.0

HPLC: RT=2.409 min, purity=98.166%

¹H NMR (400 MHz, METHANOL-d₄) δ=8.62 (d, J=2.0 Hz, 1H), 8.00 (d, J=1.2 Hz, 1H), 7.90 (dd, J=2.4, 8.4 Hz, 1H), 7.67 (d, J=8.4 Hz, 1H), 7.09 (s, 1H), 6.84-6.71 (m, 3H), 5.60-5.53 (m, 1H), 5.32-5.24 (m, 1H), 5.14-5.12 (m, 2H), 4.88-4.82 (m, 3H), 4.74-4.62 (m, 2H), 4.49-4.49 (m, 1H), 4.18-4.11 (m, 1H), 4.09-4.03 (m, 1H), 3.24-3.06 (m, 2H), 2.87-2.74 (m, 2H), 2.59-2.41 (m, 3H), 2.04 (s, 3H), 2.00-1.81 (m, 4H)

### Example A13. —Preparation of Compound 73. 2-((4-(6-((4-Cyano-2-fluorobenzyl)oxy)pyridin-2-yl)piperidin-1-yl)methyl)-4-isopropoxy-1-(thiazol-5-ylmethyl)-1H-benzo[d]imidazole-6-carboxylic acid (Compound 73) (8)

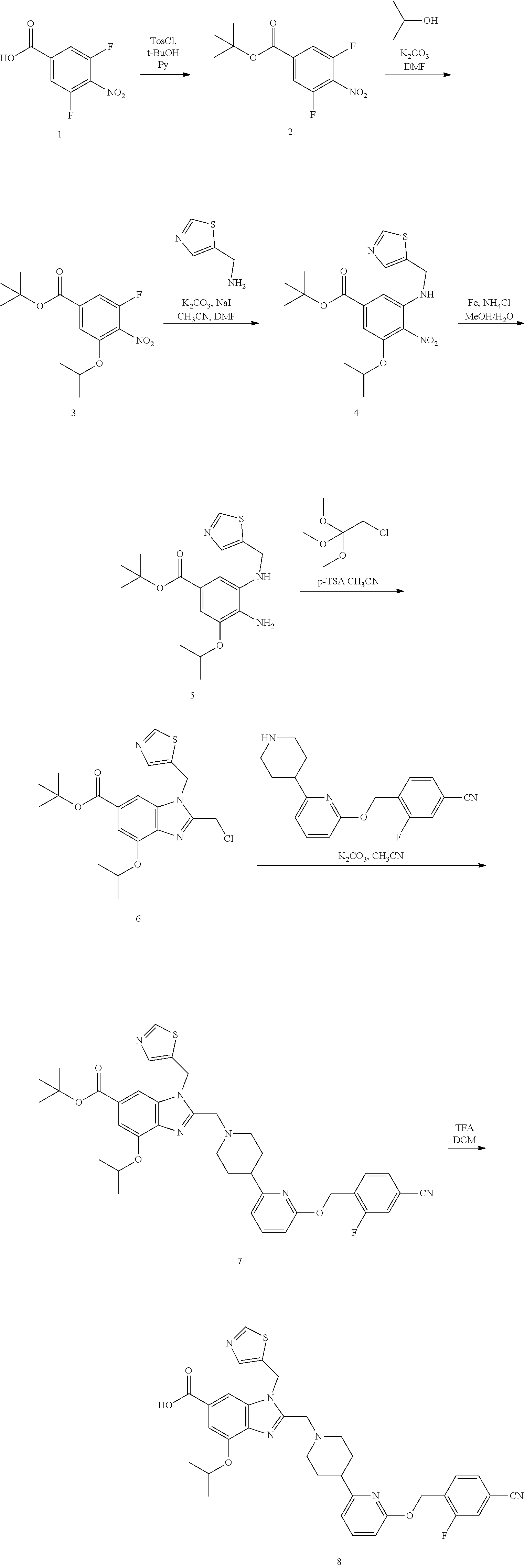

### Preparation of Intermediate (2) tert-Butyl 3,5-difluoro-4-nitrobenzoate

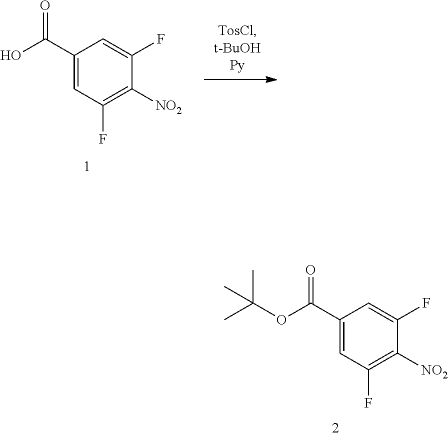 [SMILES: O=C(O)c1cc(F)c([N+](=O)[O-])c(F)c1 ; CC(C)(C)OC(=O)c1cc(F)c([N+](=O)[O-])c(F)c1]

To a solution of 3,5-difluoro-4-nitrobenzoic acid 1 (5 g, 24.6 mmol, 1 eq) in Py (10 mL) was added TosCl (9.39 g, 49.2 mmol, 2 eq). The mixture was stirred at 20° C. for 1 h. Then t-BuOH (3.65 g, 49.2 mmol, 4.71 mL, 2 eq) was added to the mixture at 20° C. The mixture was stirred at 20° C. for 16 h. LC-MS showed no starting material remained and one main peak was detected. The reaction mixture was diluted with 1M HCl to adjust pH=6, and then diluted with H₂O (20 mL) and extracted with EtOAc (50 mL*3). The combined organic layers were washed with brine (50 mL), dried over Na₂SO₄, filtered, and concentrated under reduced pressure to give a residue. The residue was purified by column chromatography (SiO₂, Petroleum ether/Ethyl acetate=1/0 to 10/1) provide product (3 g, 11.6 mmol, 47% yield) as a white solid.

¹H NMR (400 MHz, CHLOROFORM-d) δ=7.77-7.63 (m, 2H), 1.61 (s, 9H)

### Preparation of Intermediate (3) tert-Butyl 3-fluoro-5-isopropoxy-4-nitrobenzoate

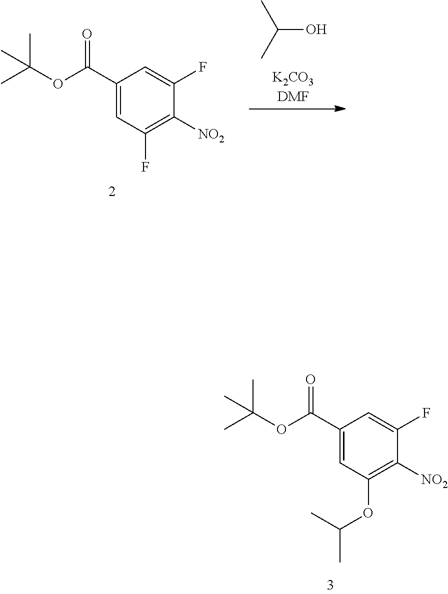

To a solution of tert-butyl 3,5-difluoro-4-nitrobenzoate 2 (1 g, 3.86 mmol, 1 eq) in i-PrOH (10 mL) was added K₂CO₃ (1.60 g, 11.6 mmol, 3 eq). The mixture was stirred at 90° C. for 12 h. TLC (Petroleum ether:Ethyl acetate=5:1, R_{f}=0.6) indicated no starting material remained and one new spot formed. The reaction mixture was diluted with H₂O (20 mL) and extracted with EtOAc (20 mL*3). The combined organic layers were washed with brine (20 mL), dried over Na₂SO₄, filtered, and concentrated under reduced pressure to give a residue. The residue was purified by column chromatography (SiO₂, Petroleum ether/Ethyl acetate=1/0 to 10/1) to obtain product (500 mg, 1.67 mmol, 43% yield) as a yellow oil.

¹H NMR (400 MHz, CHLOROFORM-d) δ=7.47 (d, J=1.2 Hz, 1H), 7.37 (dd, J=1.2, 9.2 Hz, 1H), 4.76-4.70 (m, 1H), 1.61 (s, 9H), 1.40-1.37 (m, 6H)

### Preparation of Intermediate tert-Butyl 3-isopropoxy-4-nitro-5-((thiazol-5-ylmethyl)amino)benzoate (4)

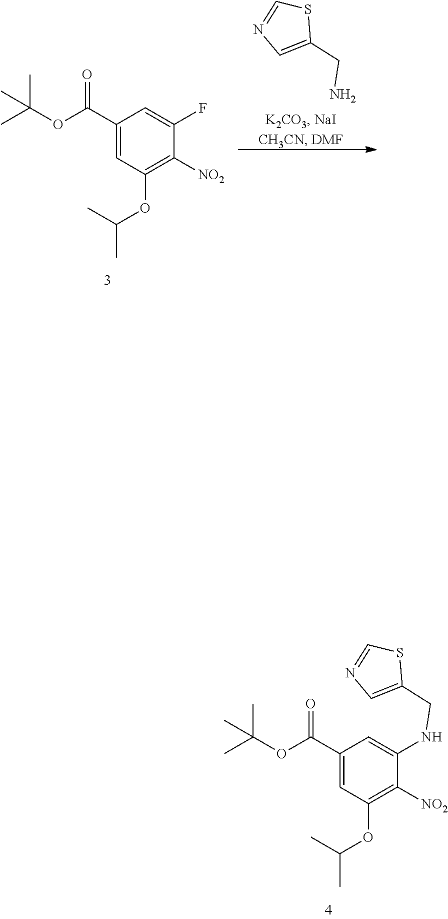 [SMILES: CC(C)Oc1cc(C(=O)OC(C)(C)C)cc(F)c1[N+](=O)[O-] ; NCc1cncs1 ; CC(C)Oc1cc(C(=O)OC(C)(C)C)cc(NCc2cncs2)c1[N+](=O)[O-]]

To a solution of tert-butyl 3-fluoro-5-isopropoxy-4-nitrobenzoate 3 (500 mg, 1.67 mmol, 1 eq) in ACN (10 mL) and DMF (1 mL) was added K₂CO₃ (924 mg, 6.68 mmol, 4 eq), NaI (501 mg, 3.34 mmol, 2 eq) and (1.56 g, 8.35 mmol, 5 eq, 2HCl). The mixture was stirred at 60° C. for 48 hr. LC-MS showed ˜39% of starting material remained. Several new peaks were shown on LC-MS and ˜36% of desired product was detected. The reaction mixture was diluted with H₂O (20 mL) and extracted with EtOAc (20 mL*3). The combined organic layers were washed with brine (20 mL), dried over Na₂SO₄, filtered, and concentrated under reduced pressure to give a residue. The residue was purified by column chromatography (SiO₂, Petroleum ether/Ethyl acetate=1/0 to 1/1) to provide product (210 mg, 534 μmol, 31% yield) as a yellow oil.

### Preparation of Intermediate (5) tert-Butyl 4-amino-3-isopropoxy-5-((thiazol-5-ylmethyl)amino)benzoate

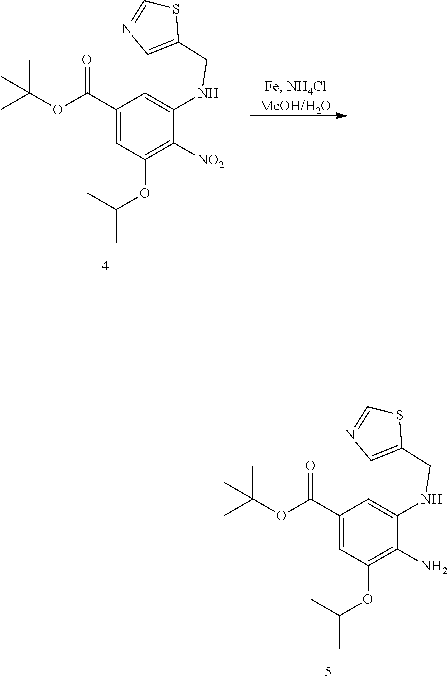 [SMILES: CC(C)Oc1cc(C(=O)OC(C)(C)C)cc(NCc2cncs2)c1[N+](=O)[O-] ; CC(C)Oc1cc(C(=O)OC(C)(C)C)cc(NCc2cncs2)c1N]

To a solution of tert-butyl 3-isopropoxy-4-nitro-5-((thiazol-5-ylmethyl)amino)benzoate 4 (100 mg, 254 μmol, 1 eq) in MeOH (4 mL) and H₂O (2 mL) was added Fe (71 mg, 1.27 mmol, 5 eq) and NH₄Cl (68 mg, 1.27 mmol, 5 eq). The mixture was stirred at 70° C. for 1 hr. LC-MS showed starting materuak was consumed completely and one main peak with desired m/z was detected. The reaction mixture was filtered through a pad of Celite and filter cake was washed with MeOH (5 mL*3). The filtrate was concentrated to give a residue. The residue was dissolved with EtOAc (20 mL) and washed with H₂O (5 mL*3), brine (10 mL*2), dried over Na₂SO₄, filtered and concentrated under reduced pressure to give a residue. The residue was purified by column chromatography (SiO₂, Petroleum ether/Ethyl acetate=1/0 to 0/1). 5 (80 mg, 220.10 μmol, 86% yield) was obtained as a yellow oil.

LCMS: RT=0.492 min, MS cal.: 363.2, [M−55]⁺=308.3

¹H NMR (400 MHz, CHLOROFORM-d) δ=8.76 (s, 1H), 7.86 (s, 1H), 7.19 (s, 1H), 7.13 (s, 1H), 4.67-4.54 (m, 3H), 1.58 (s, 9H), 1.37 (d, J=6.0 Hz, 6H)

### Preparation of Intermediate (6) tert-Butyl 2-(chloromethyl)-4-isopropoxy-1-(thiazol-5-ylmethyl)-1H-benzo[d]imidazole-6-carboxylate

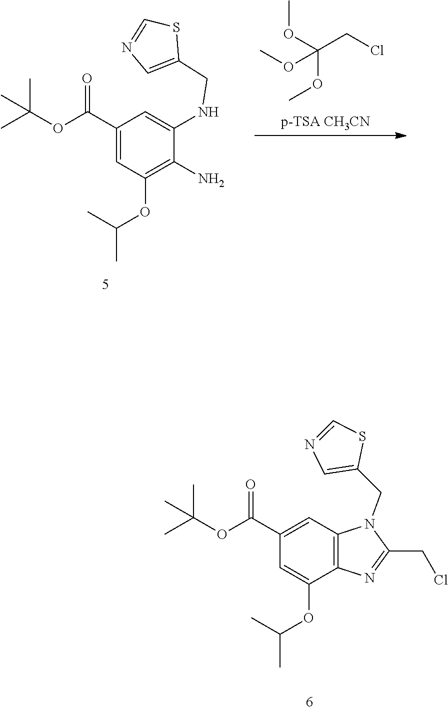

To a solution of tert-butyl 4-amino-3-isopropoxy-5-((thiazol-5-ylmethyl)amino)benzoate 5 (70 mg, 193 μmol, 1 eq) in ACN (3 mL) was added p-TSA (3.66 mg, 19.3 μmol, 0.1 eq) and 2-chloro-1,1,1-trimethoxyethane (35.7 mg, 231 μmol, 31.2 μL, 1.2 eq). The mixture was stirred at 60° C. for 2 h. LC-MS showed ˜10% of starting material remained. Several new peaks were shown on LC-MS and ˜70% of desired compound was detected. The reaction mixture was concentrated under reduced pressure to provide product (70 mg, 166 μmol, 86% yield) as a yellow oil.

LCMS: RT=0.528 min, MS cal.: 421.1, 423.1, [M+H]⁺=422.2, 424.2

### Preparation of Intermediate tert-Butyl 2-((4-(6-((4-cyano-2-fluorobenzyl)oxy)pyridin-2-yl)piperidin-1-yl)methyl)-4-isopropoxy-1-(thiazol-5-ylmethyl)-1H-benzo[d]imidazole-6-carboxylate (7)

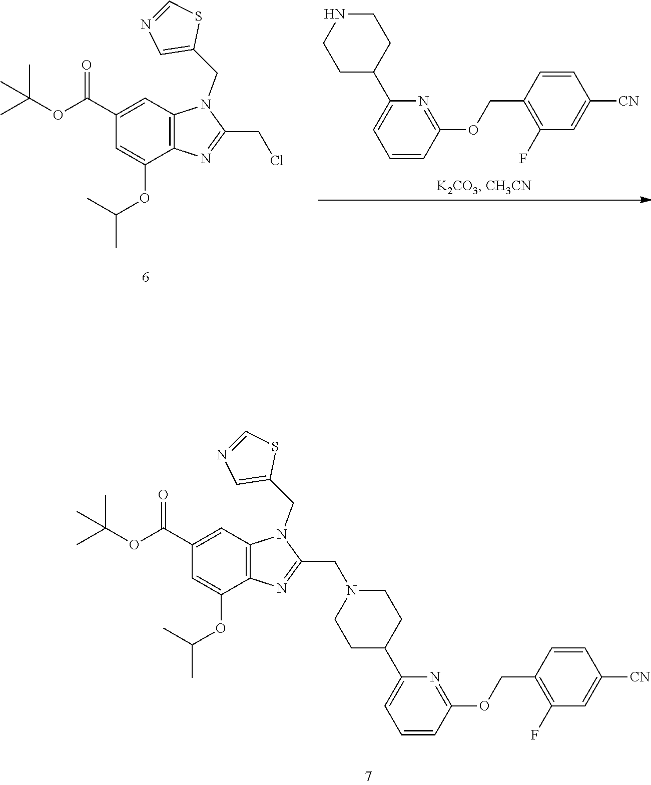 [SMILES: CC(C)Oc1cc(C(=O)OC(C)(C)C)cc2c1nc(CCl)n2Cc1cncs1 ; N#Cc1ccc(COc2cccc(C3CCNCC3)n2)c(F)c1 ; CC(C)Oc1cc(C(=O)OC(C)(C)C)cc2c1nc(CN1CCC(c3cccc(OCc4ccc(C#N)cc4F)n3)CC1)n2Cc1cncs1]

To a solution of tert-butyl 2-(chloromethyl)-4-isopropoxy-1-(thiazol-5-ylmethyl)-1H-benzo[d]imidazole-6-carboxylate 6 (60 mg, 142 μmol, 1 eq) in ACN (3 mL) was added K₂CO₃ (59 mg, 427 μmol, 3 eq) and 3-fluoro-4-(((6-(piperidin-4-yl)pyridin-2-yl)oxy)methyl)benzonitrile (44 mg, 142 μmol, 1 eq). The mixture was stirred at 60° C. for 4 h. LC-MS showed starting material was consumed completely and one main peak with desired m/z was detected. The reaction mixture was diluted with H₂O (10 mL) and extracted with EtOAc (10 mL*3). The combined organic layers were washed with brine (10 mL), dried over Na₂SO₄, filtered, and concentrated under reduced pressure to give a residue. The residue was purified by column chromatography (SiO₂, Petroleum ether/Ethyl acetate=1/0 to 0/1) to provide product (80 mg, 115 μmol, 80% yield) as a yellow solid.

LCMS: RT=0.514 min, MS cal.: 696.3, [M+H]⁺=697.2

### Preparation of 2-((4-(6-((4-Cyano-2-fluorobenzyl)oxy)pyridin-2-yl)piperidin-1-yl)methyl)-4-isopropoxy-1-(thiazol-5-ylmethyl)-1H-benzo[d]imidazole-6-carboxylic acid, Compound 73 (8)

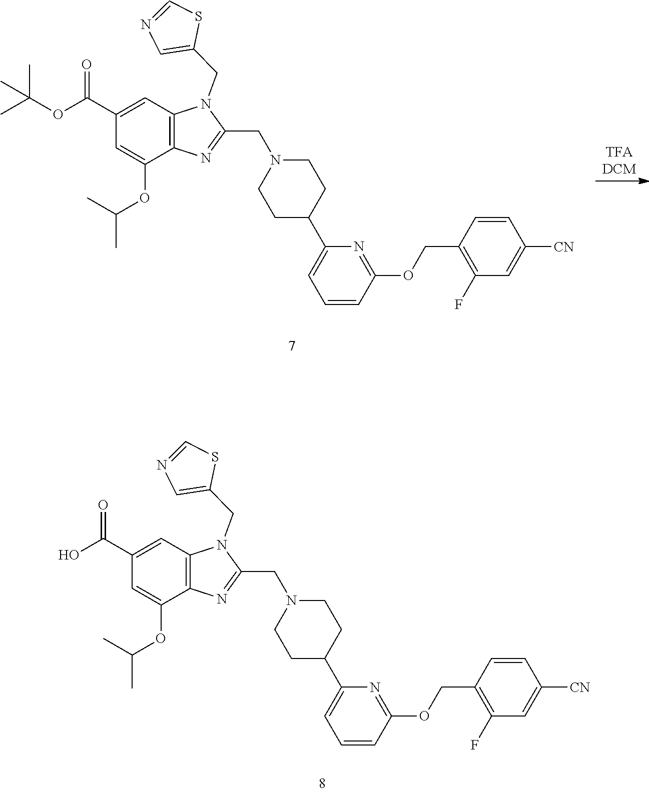 [SMILES: CC(C)Oc1cc(C(=O)OC(C)(C)C)cc2c1nc(CN1CCC(c3cccc(OCc4ccc(C#N)cc4F)n3)CC1)n2Cc1cncs1 ; CC(C)Oc1cc(C(=O)O)cc2c1nc(CN1CCC(c3cccc(OCc4ccc(C#N)cc4F)n3)CC1)n2Cc1cncs1]

To a solution of tert-butyl 2-((4-(6-((4-cyano-2-fluorobenzyl)oxy)pyridin-2-yl)piperidin-1-yl)methyl)-4-isopropoxy-1-(thiazol-5-ylmethyl)-1H-benzo[d]imidazole-6-carboxylate 7 (75 mg, 108 μmol, 1 eq) in DCM (4 mL) was added TFA (1.54 g, 13.5 mmol, 1 mL, 125 eq) at 0° C. The mixture was stirred at 0° C. for 15 min and then stirred at 20° C. for 12 h. LC-MS showed starting material was consumed completely and one main peak with desired m/z was detected. The reaction mixture was added saturated aq. NaHCO₃ (5 mL) to adjust pH=7, and then diluted with H₂O (1 mL) and extracted with DCM (3 mL*3). The combined organic layers were washed with brine (3 mL), dried over Na₂SO₄, filtered, and concentrated under reduced pressure to give a residue. The residue was purified by prep-HPLC (neutral condition; column: Waters Xbridge BEH C18 100*30 mm*10 um; mobile phase: [H₂O (10 mM NH₄HCO₃)-ACN]; gradient: 10%-40% B over 8.0 min) to provide product (17.8 mg, 27.7 μmol, 26% yield) as a white solid.

LCMS: RT=2.346 min, MS cal.: 640.2, [M+H]⁺=641.3

HPLC: Rt=10.660 min, purity: 100%

¹H NMR (400 MHz, DMSO-d6) δ=8.97 (s, 1H), 8.01 (s, 1H), 7.88 (d, J=10.4 Hz, 1H), 7.77 (d, J=1.2 Hz, 1H), 7.72-7.69 (m, 2H), 7.67-7.62 (m, 1H), 7.25 (s, 1H), 6.85 (d, J=7.2 Hz, 1H), 6.71 (d, J=8.4 Hz, 1H), 5.92 (s, 2H), 5.47 (s, 2H), 5.04-4.95 (m, 1H), 3.84 (s, 2H), 2.96-2.93 (m, 2H), 2.63-2.53 (m, 1H), 2.20-2.16 (m, 2H), 1.77-1.60 (m, 4H), 1.34 (d, J=6.0 Hz, 6H)

### Example A14. —Preparation of Compound 74. (S)-2-((4-(2-(5-Chloropyridin-2-yl)-2-methylbenzo[d][1,3]dioxol-4-yl)piperidin-1-yl)methyl)-4-(2-methoxyethoxy)-1H-benzo[d]imidazole-6-carboxylic acid (Compound 74) (7)

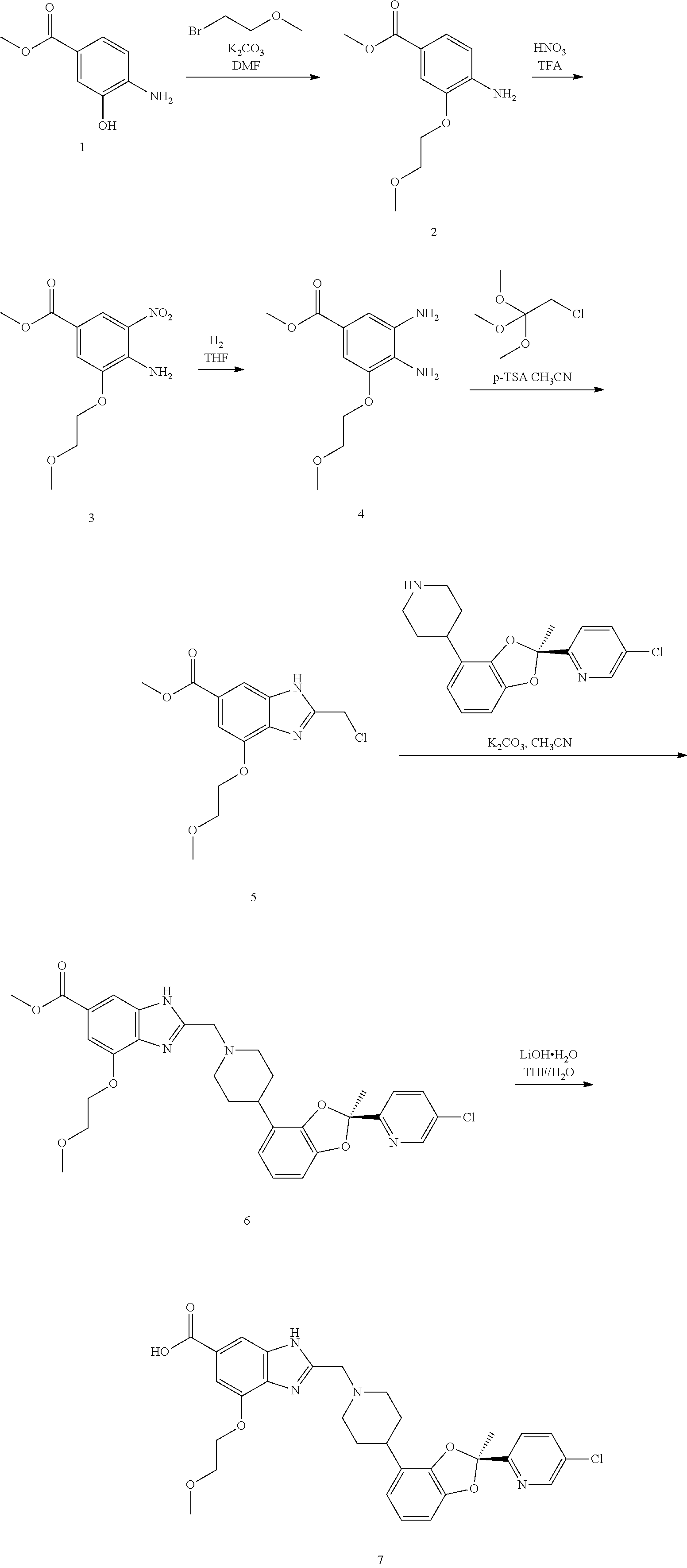

### Preparation of Intermediate Methyl 4-amino-3-(2-methoxyethoxy)benzoate (2)

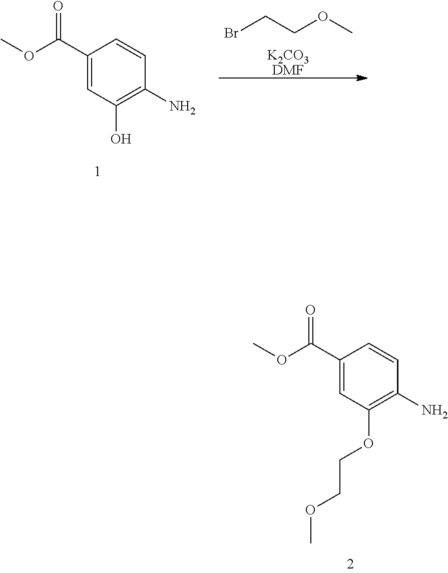

Methyl 4-amino-3-hydroxybenzoate 1 (5 g, 29.9 mmol, 1 eq), 1-bromo-2-methoxyethane (4.16 g, 29.9 mmol, 2.81 mL, 1 eq) and K₂CO₃ (12.4 g, 89.7 mmol, 3 eq) in DMF (50 mL) were degassed and purged with N₂ for 3 times, and then the mixture was stirred at 100° C. for 12 h under N₂ atmosphere. LCMS showed starting material was consumed completely. The reaction mixture was diluted with H₂O (60 mL) and extracted with EtOAc (100 mL*3). The combined organic layers were washed with brine (50 mL*3), dried over Na₂SO₄, filtered, and concentrated under reduced pressure to give a residue. The residue was purified by column chromatography (SiO₂, Petroleum ether/Ethyl acetate=100/1 to 1/1) to provide product (4.81 g, 21.4 mmol, 71% yield) as a black solid.

LCMS: RT=1.111 min, MS cal.: 225.2, [M+H]⁺=226.2

¹H NMR (400 MHz, DMSO-d₆) δ=7.39 (dd, J=1.6, 8.4 Hz, 1H), 7.31 (s, 1H), 6.66 (d, J=8.4 Hz, 1H), 5.57 (br s, 2H), 4.12-4.07 (m, 2H), 3.77 (s, 3H), 3.69-3.66 (m, 2H), 3.32 (s, 3H)

### Preparation of Intermediate Methyl 4-amino-3-(2-methoxyethoxy)-5-nitrobenzoate (3)

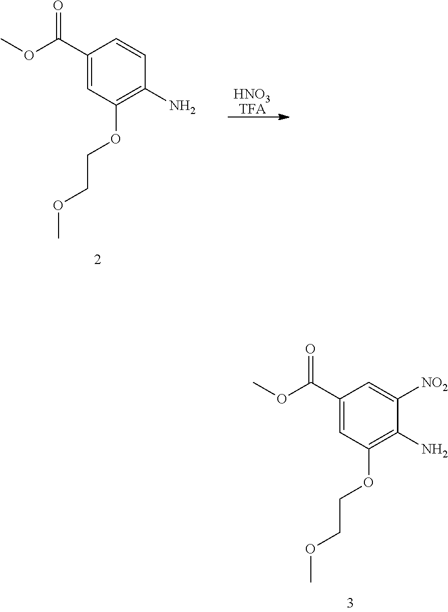 [SMILES: COCCOc1cc(C(=O)OC)ccc1N ; COCCOc1cc(C(=O)OC)cc([N+](=O)[O-])c1N ; "HNO3TFA"]

Equip a 250 mL three-necked round bottom flask and thermometer, N₂ balloon. TFA (38 mL) was charged to the 250 mL three-necked round bottom flask, then methyl 4-amino-3-(2-methoxyethoxy)benzoate 2 (3.8 g, 16.9 mmol, 1 eq) was added at 20° C. for 2 min. At 0° C. inner temperature, KNO₃ (2.56 g, 25.3 mmol, 1.5 eq) was added in portions to the reaction mixture at 0° C. within 8 min. After the addition, the mixture was stirred at 0° C. for 3 h. LCMS showed the starting material was consumed completely. After 3 h, the reaction mixture was added dropwise to H₂O (60 mL) at 0° C. within 10 min. Then the reaction mixture was filtered and the filter cake was dried in vacuum to give the crude product. The filtrate was extracted by EtOAc (80 mL*3). Then organic phase was combined and washed by H₂O (60 mL), brine (30 mL), dried with Na₂SO₄, filtered. The organic phase was concentrated under reduced pressure at 40° C. to give a residue. The residue was purified by column chromatography (SiO₂, Petroleum ether/Ethyl acetate=100/1 to 1/1) to provide product (3.36 g, 12.4 mmol, 74% yield) as a yellow solid.

LCMS: RT=1.366 min, MS cal.: 270.2, [M+H]⁺=271.3

¹H NMR (400 MHz, DMSO-d₆) δ=8.27 (d, J=1.6 Hz, 1H), 7.56 (br s, 2H), 7.46 (d, J=1.6 Hz, 1H), 4.27-4.25 (m, 2H), 3.84 (s, 3H), 3.78-3.74 (m, 2H), 3.33 (s, 3H)

### Preparation of Intermediate Methyl 3,4-diamino-5-(2-methoxyethoxy)benzoate (4)

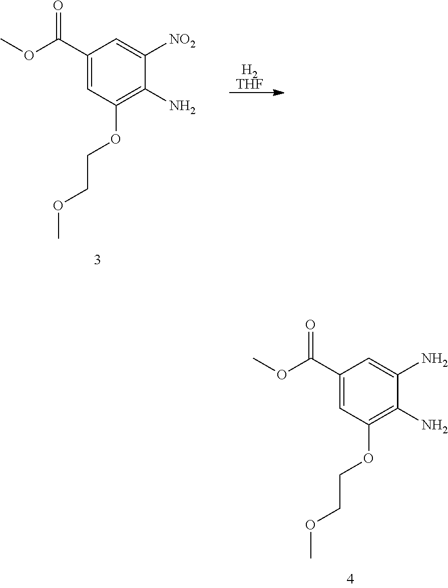 [SMILES: COCCOc1cc(C(=O)OC)cc([N+](=O)[O-])c1N ; COCCOc1cc(C(=O)OC)cc(N)c1N ; "H2THF"]

Equip a 50 mL round bottom flask, H2 (15 psi) atmosphere. THF (15 mL) was charged to the round bottom flask, then methyl 4-amino-3-(2-methoxyethoxy)-5-nitrobenzoate 3 (1 g, 3.70 mmol, 1 eq) was added to the mixture at 20° C. for 1 min. At 20° C. (inner temperature), Pd/C (473 mg, 10%) was added to the reaction mixture at 20° C. under H₂ atmosphere. After the addition, the suspension was degassed and purged with H₂ for 3 times, and then the mixture was stirred under H₂ atmosphere at 20° C. for 1 h. LCMS showed the starting material was consumed completely. The reaction mixture was diluted with THF (20 ml), and then filtered through celite pad. The filter cake was rinsed with THF (20 ml*3), and the filtrate was concentrated to provide product (889 mg, crude) as a yellow solid.

LCMS: RT=1.095 min, MS cal.: 240.3, [M+H]⁺=241.2

¹H NMR (400 MHz, DMSO-d₆) δ=6.98 (d, J=1.6 Hz, 1H), 6.85 (d, J=1.6 Hz, 1H), 4.82 (br s, 4H), 4.06-4.04 (m, 2H), 3.75 (s, 3H), 3.70-3.64 (m, 2H), 3.33 (s, 3H)

### Preparation of Intermediate Methyl 2-(chloromethyl)-4-(2-methoxyethoxy)-1H-benzo[d]imidazole-6-carboxylate (5)

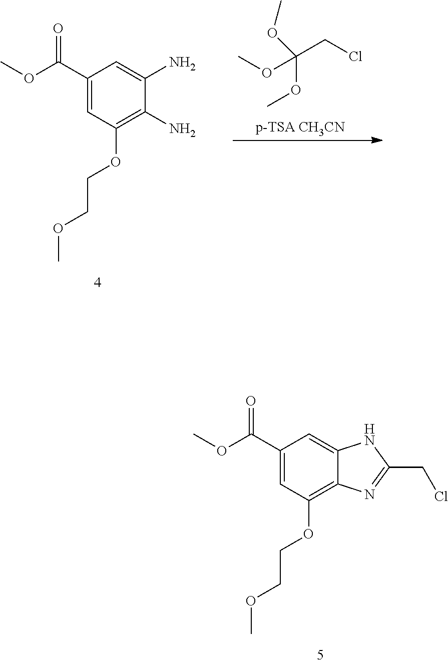

A mixture of methyl 3,4-diamino-5-(2-methoxyethoxy)benzoate 4 (880 mg, 3.66 mmol, 1 eq), 2-chloro-1,1,1-trimethoxyethane (1.70 g, 11 mmol, 1.48 mL, 3 eq), p-TSA (315 mg, 1.83 mmol, 0.5 eq) in ACN (10 mL) was degassed and purged with N₂ for 3 times, and then the mixture was stirred at 60° C. for 2 h under N₂ atmosphere. LCMS showed the starting material was consumed completely. The reaction mixture was filtered and concentrated under reduced pressure to provide product (1 g, crude) as a yellow oil.

LCMS: RT=1.325 min, MS cal.: 298.7, [M+H]⁺=299.1

### Preparation of Intermediate Methyl (S)-2-((4-(2-(5-chloropyridin-2-yl)-2-methylbenzo[d][1,3]dioxol-4-yl)piperidin-1-yl)methyl)-4-(2-methoxyethoxy)-1H-benzo[d]imidazole-6-carboxylate (6)

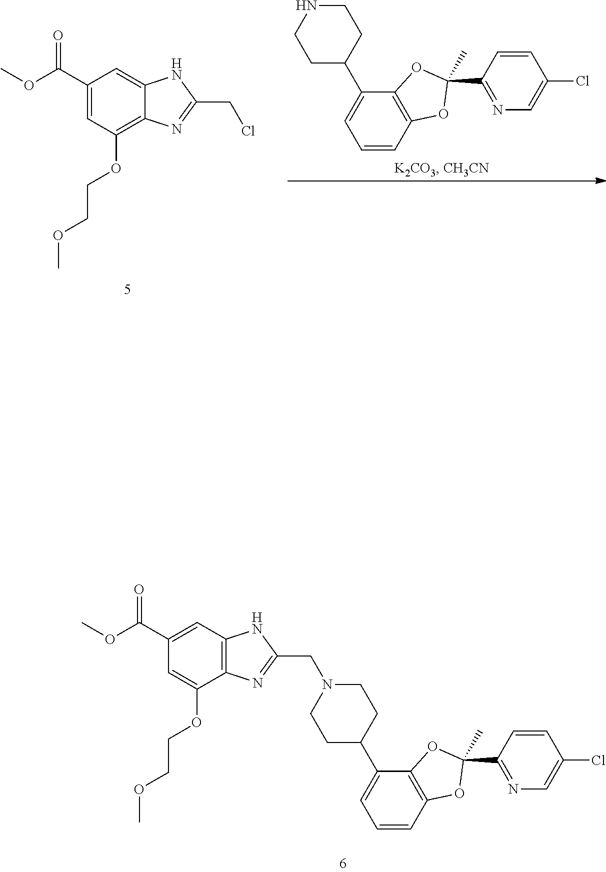

A mixture of methyl 2-(chloromethyl)-4-(2-methoxyethoxy)-1H-benzo[d]imidazole-6-carboxylate 5 (1 g, 3.35 mmol, 1 eq), (S)-5-chloro-2-(2-methyl-4-(piperidin-4-yl)benzo[d][1,3]dioxol-2-yl)pyridine (1.11 g, 3.35 mmol, 1 eq), K₂CO₃ (1.39 g, 10.04 mmol, 3 eq) in ACN (15 mL) was degassed and purged with N₂ for 3 times, and then the mixture was stirred at 60° C. for 4 h under N₂ atmosphere. LCMS showed the starting material was consumed completely. The reaction mixture was diluted with H₂O (10 mL) and extracted with EtOAc (20 mL*3). The combined organic layers were washed with brine (10 mL*2), dried over Na₂SO₄, filtered, and concentrated under reduced pressure to give a residue. The residue was purified by column chromatography (SiO₂, Ethyl acetate/MeOH=100/1 to 1/1) to provide product (800 mg, 1.35 mmol, 40% yield) as a yellow solid.

LCMS: RT=2.194 min, MS cal.: 593.1, [M+H]⁺=593.2

### Preparation of (S)-2-((4-(2-(5-Chloropyridin-2-yl)-2-methylbenzo[d][1,3]dioxol-4-yl)piperidin-1-yl)methyl)-4-(2-methoxyethoxy)-1H-benzo[d]imidazole-6-carboxylic acid, Compound 74 (7)

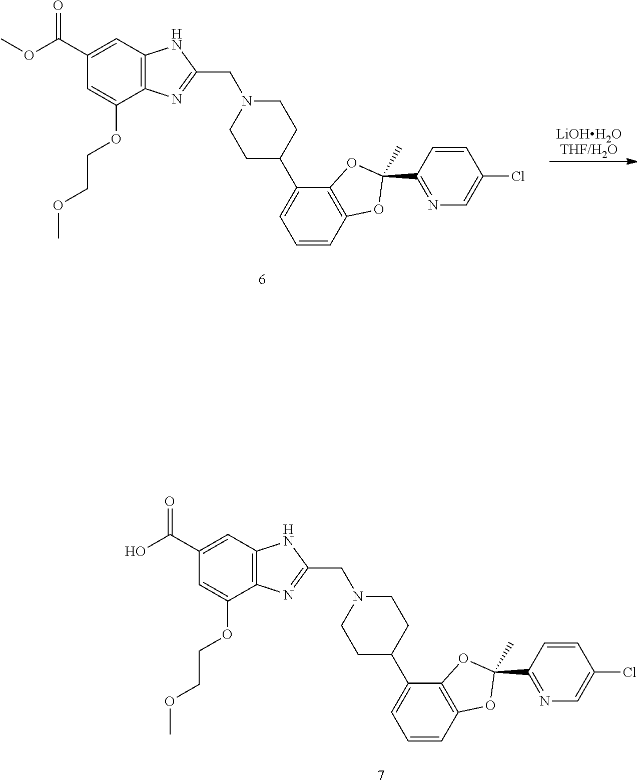 [SMILES: COCCOc1cc(C(=O)OC)cc2[nH]c(CN3CCC(c4cccc5c4O[C@@](C)(c4ccc(Cl)cn4)O5)CC3)nc12 ; "LiOH·H2OTHF/H2O" ; COCCOc1cc(C(=O)O)cc2[nH]c(CN3CCC(c4cccc5c4O[C@@](C)(c4ccc(Cl)cn4)O5)CC3)nc12]

A mixture of methyl (S)-2-((4-(2-(5-chloropyridin-2-yl)-2-methylbenzo[d][1,3]dioxol-4-yl)piperidin-1-yl)methyl)-4-(2-methoxyethoxy)-1H-benzo[d]imidazole-6-carboxylate 6 (60 mg, 101.17 μmol, 1 eq), LiOH·H₂O (6.37 mg, 151.75 μmol, 1.5 eq) in THF (0.7 mL) and H₂O (0.3 mL) was degassed and purged with N₂ for 3 times, and then the mixture was stirred at 25° C. for 48 h under N₂ atmosphere. LCMS showed the starting material was consumed completely. The crude product was purified by reversed-phase HPLC (column: Waters Xbridge Prep OBD C18 150*40 mm*10 um; mobile phase: [H₂O (10 mM NH₄HCO₃)-ACN]; gradient: 25%-55% B over 8.0 min) to provide product (20.06 mg, 34.64 μmol, 34.24% yield) as a white solid.

LCMS: RT=2.514 min, MS cal.: 579.1, [M+H]⁺=579.0

HPLC: RT=10.013 min, purity=99.829%

SFC: ee=100%

¹H NMR (400 MHz, METHANOL-d₄) δ=8.61 (d, J=2.0 Hz, 1H), 7.95 (s, 1H), 7.88 (dd, J=2.4, 8.4 Hz, 1H), 7.66 (d, J=8.4 Hz, 1H), 7.45 (s, 1H), 6.82-6.77 (m, 1H), 6.72 (s, 2H), 4.41-4.32 (m, 2H), 3.91 (s, 2H), 3.90-3.86 (m, 2H), 3.46 (s, 3H), 3.11-3.09 (m, 2H), 2.79-2.63 (m, 1H), 2.37-2.33 (m, 2H), 2.07-1.92 (m, 5H), 1.76 (s, 2H)

### Example A15. —Preparation of Compound 76. (S)-2-((4-(2-(5-Chloropyridin-2-yl)-2-methylbenzo[d][1,3]dioxol-4-yl)piperidin-1-yl)methyl)-7-(2-methoxyethoxy)-1-methyl-1H-benzo[d]imidazole-5-carboxylic acid (Compound 76) (4)

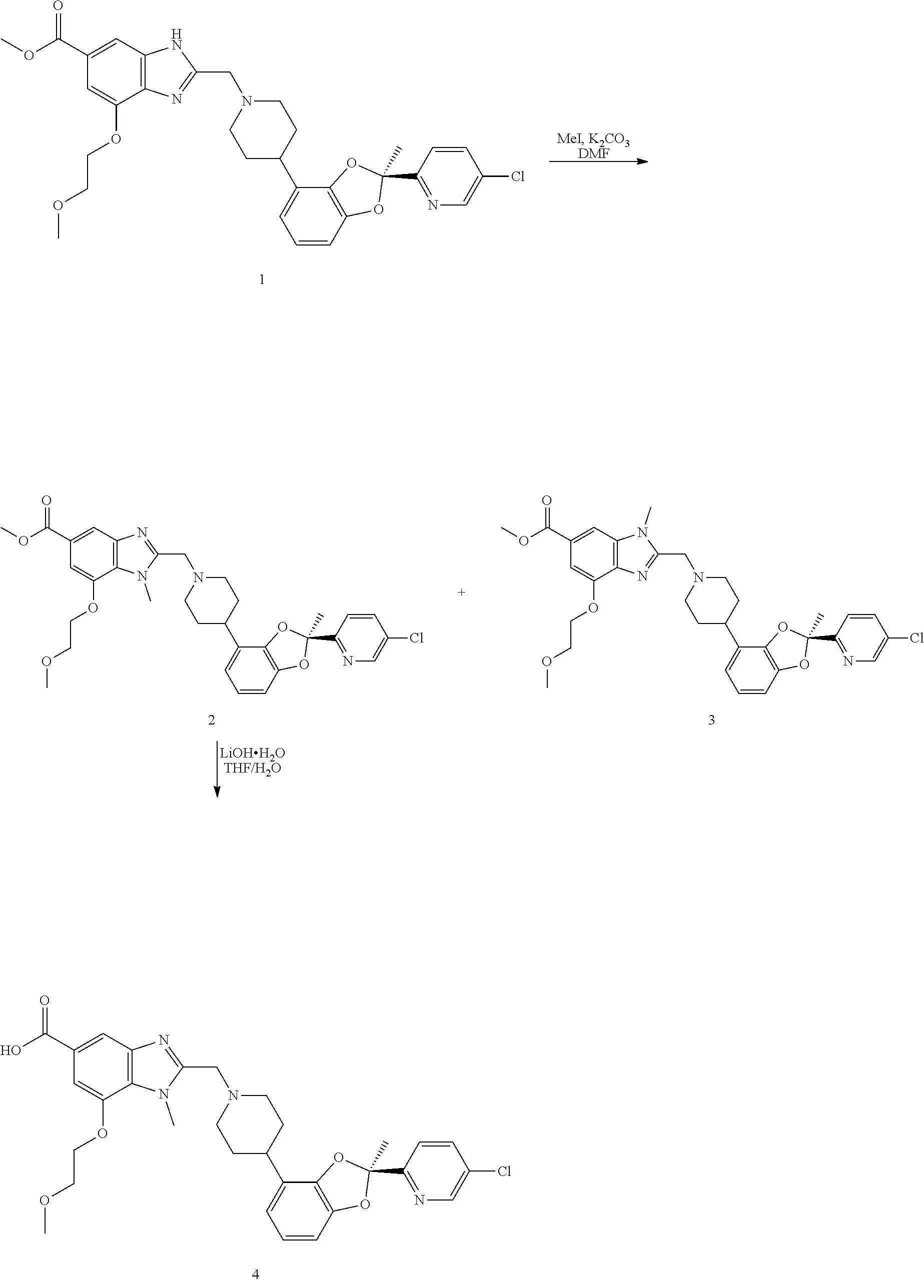

### Preparation of Intermediate Methyl (S)-2-((4-(2-(5-chloropyridin-2-yl)-2-methylbenzo[d][1,3]dioxol-4-yl)piperidin-1-yl)methyl)-7-(2-methoxyethoxy)-1-methyl-1H-benzo[d]imidazole-5-carboxylate (2) and Methyl (S)-2-((4-(2-(5-chloropyridin-2-yl)-2-methylbenzo[d][1,3]dioxol-4-yl)piperidin-1-yl)methyl)-4-(2-methoxyethoxy)-1-methyl-1H-benzo[d]imidazole-6-carboxylate (3)

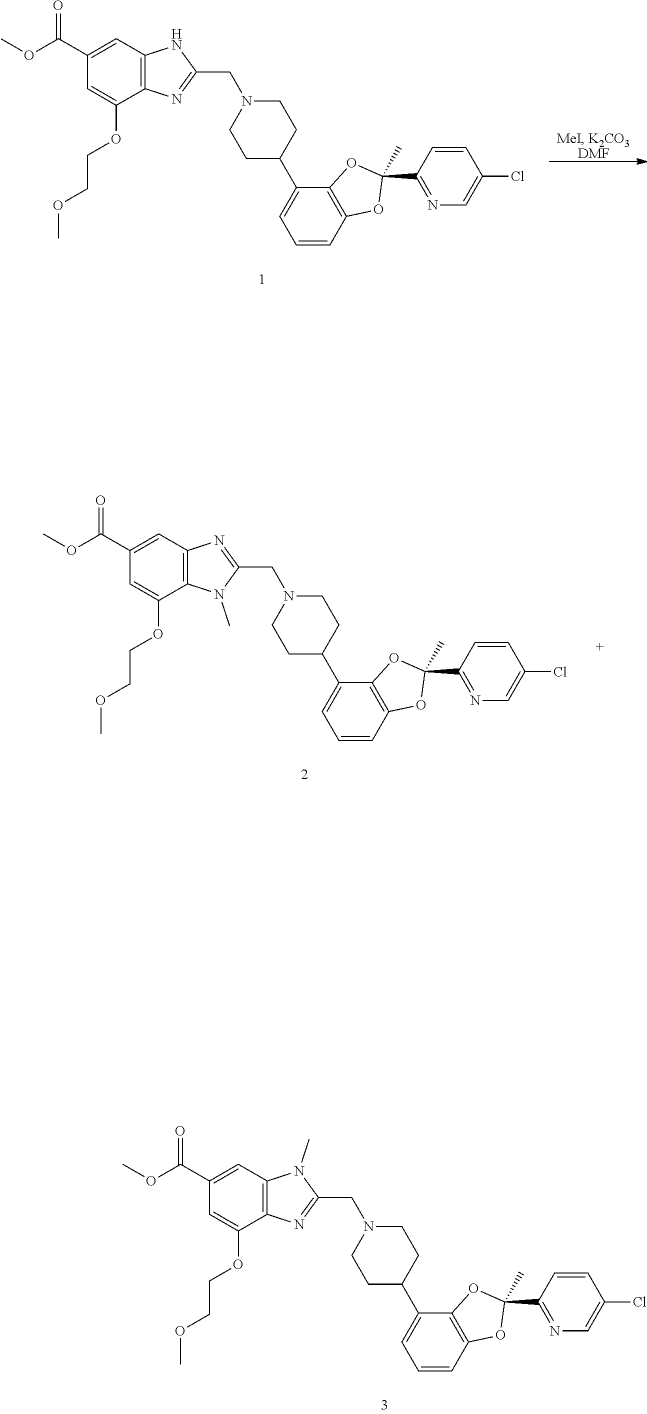

7 reactions were carried out in parallel. DMF (2 mL) was charged to the three-necked round bottom flask, then methyl (S)-2-((4-(2-(5-chloropyridin-2-yl)-2-methylbenzo[d][1,3]dioxol-4-yl)piperidin-1-yl)methyl)-7-(2-methoxyethoxy)-1-methyl-1H-benzo[d]imidazole-5-carboxylate (100 mg, 169 μmol, 1 eq) was added at 20° C. for 0.1 h. At 0° C., CH₃I (23.9 mg, 169 μmol, 10.5 μL, 1 eq) in DMF (1 mL) was added dropwise to the reaction mixture at 0° C. within 0.1 h. After the addition, the mixture was stirred at 20° C. for 12 h. LCMS showed starting material was consumed completely and desired mass was detected. After 12 h, the 7 reactions were combined for work up. The reaction mixture was added dropwise to H₂O (35 mL) at 0° C. The mixture was extracted by EtOAc (30 mL). Then organic phase was washed by H₂O (40 mL), brine (40 mL), dried with Na₂SO₄, filtered. The organic phase was concentrated under reduced pressure at 45° C. to give a residue. The crude product was purified by prep-HPLC (column: Waters Xbridge Prep OBD C18 150*40 mm*10 um; mobile phase: [H₂O (10 mM NH₄HCO₃)-ACN]; gradient: 50%-80% B over 8.0 min). Methyl (S)-2-((4-(2-(5-chloropyridin-2-yl)-2-methylbenzo[d][1,3]dioxol-4-yl)piperidin-1-yl)methyl)-7-(2-methoxyethoxy)-1-methyl-1H-benzo[d]imidazole-5-carboxylate (2, 85 mg, 140 μmol, 12% yield) was obtained as a white solid. Methyl (S)-2-((4-(2-(5-chloropyridin-2-yl)-2-methylbenzo[d][1,3]dioxol-4-yl)piperidin-1-yl)methyl)-4-(2-methoxyethoxy)-1-methyl-1H-benzo[d]imidazole-6-carboxylate (3, 75 mg, 124 μmol, 10% yield) was obtained as a white solid.

LCMS: RT (2)=2.463 min, MS cal.: 607.10, [M+H]⁺=607.2

¹H NMR (400 MHz, CHLOROFORM-d) δ=8.62 (d, J=2.0 Hz, 1H), 8.08 (s, 1H), 7.68 (dd, J=2.4, 8.4 Hz, 1H), 7.58 (d, J=8.4 Hz, 1H), 7.40 (s, 1H), 6.81-6.77 (m, 1H), 6.73-6.70 (m, 2H), 4.36-4.30 (m, 2H), 4.21 (s, 3H), 3.93 (s, 3H), 3.90-3.75 (m, 4H), 3.48 (s, 3H), 3.03-3.01 (m, 2H), 2.77-2.75 (m, 1H), 2.47-2.19 (m, 2H), 2.05 (s, 3H), 1.96-1.75 (m, 4H)

LCMS: RT (3)=2.361 min, MS cal.: 607.10, [M+H]⁺=607.2

¹H NMR (400 MHz, CHLOROFORM-d) δ=8.62 (d, J=1.6 Hz, 1H), 7.78 (s, 1H), 7.72-7.65 (m, 1H), 7.63-7.52 (m, 1H), 7.44 (s, 1H), 6.82-6.68 (m, 3H), 4.45-4.43 (m, 2H), 3.96 (s, 6H), 3.95-3.81 (m, 4H), 3.47 (s, 3H), 3.10-2.84 (m, 2H), 2.84-2.69 (m, 1H), 2.32-2.22 (m, 2H), 2.04 (s, 3H), 1.98-1.76 (m, 4H)

### Preparation of (S)-2-((4-(2-(5-Chloropyridin-2-yl)-2-methylbenzo[d][1,3]dioxol-4-yl)piperidin-1-yl)methyl)-7-(2-methoxyethoxy)-1-methyl-1H-benzo[d]imidazole-5-carboxylic acid, Compound 76 (4)

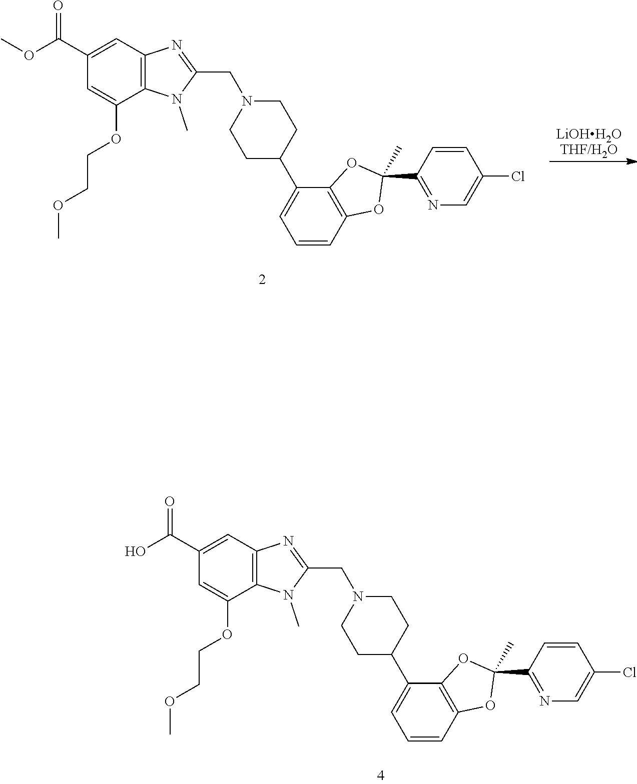 [SMILES: COCCOc1cc(C(=O)OC)cc2nc(CN3CCC(c4cccc5c4O[C@@](C)(c4ccc(Cl)cn4)O5)CC3)n(C)c12 ; "LiOH·H2OTHF/H2O" ; COCCOc1cc(C(=O)O)cc2nc(CN3CCC(c4cccc5c4O[C@@](C)(c4ccc(Cl)cn4)O5)CC3)n(C)c12]

A mixture of methyl (S)-2-((4-(2-(5-chloropyridin-2-yl)-2-methylbenzo[d][1,3]dioxol-4-yl)piperidin-1-yl)methyl)-7-(2-methoxyethoxy)-1-methyl-1H-benzo[d]imidazole-5-carboxylate 2 (60 mg, 101 μmol, 1 eq), LiOH·H₂O (6.37 mg, 152 μmol, 1.5 eq) in THF (0.7 mL) and H₂O (0.3 mL) was degassed and purged with N₂ for 3 times, and then the mixture was stirred at 25° C. for 48 h under N₂ atmosphere. LCMS showed the starting material was consumed completely. The crude product was purified by reversed-phase HPLC (column: Waters Xbridge Prep OBD C18 150*40 mm*10 um; mobile phase: [H₂O (10 mM NH₄HCO₃)-ACN]; gradient: 25%-55% B over 8.0 min) to provide product (20.06 mg, 34.6 μmol, 34% yield) as a white solid.

LCMS: RT=2.514 min, MS cal.: 579.1, [M+H]⁺=579.0

HPLC: RT=10.013 min, purity=99.829%

SFC: ee=100%

¹H NMR (400 MHz, METHANOL-d₄) δ=8.61 (d, J=2.0 Hz, 1H), 7.95 (s, 1H), 7.88 (dd, J=2.4, 8.4 Hz, 1H), 7.66 (d, J=8.4 Hz, 1H), 7.45 (s, 1H), 6.82-6.77 (m, 1H), 6.72 (s, 2H), 4.41-4.32 (m, 2H), 3.91 (s, 2H), 3.90-3.86 (m, 2H), 3.46 (s, 3H), 3.11-3.09 (m, 2H), 2.79-2.63 (m, 1H), 2.37-2.33 (m, 2H), 2.07-1.92 (m, 5H), 1.76 (s, 2H).

### Example A16. —Preparation of Compound 30. (S)-2-((4-(2-(5-Chloropyridin-2-yl)-2-methylbenzo[d][1,3]dioxol-4-yl)piperidin-1-yl)methyl)-4-(2-methoxyethoxy)-1-methyl-1H-benzo[d]imidazole-6-carboxylic acid (Compound 30) (2)

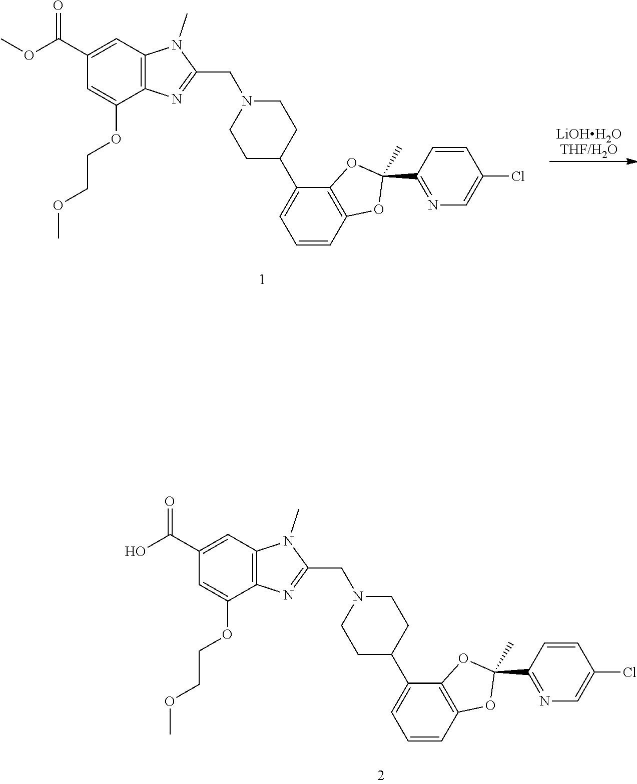 [SMILES: COCCOc1cc(C(=O)OC)cc2c1nc(CN1CCC(c3cccc4c3O[C@@](C)(c3ccc(Cl)cn3)O4)CC1)n2C ; "LiOH·H2OTHF/H2O" ; COCCOc1cc(C(=O)O)cc2c1nc(CN1CCC(c3cccc4c3O[C@@](C)(c3ccc(Cl)cn3)O4)CC1)n2C]

A mixture of methyl (S)-2-((4-(2-(5-Chloropyridin-2-yl)-2-methylbenzo[d][1,3]dioxol-4-yl)piperidin-1-yl)methyl)-4-(2-methoxyethoxy)-1-methyl-1H-benzo[d]imidazole-6-carboxylate 1 (70 mg, 115 μmol, 1 eq), LiOH·H₂O (24.2 mg, 577 μmol, 5 eq) in THF (0.5 mL) and H₂O (0.5 mL) was degassed and purged with N₂ for 3 times, and then the mixture was stirred at 25° C. for 12 h under N₂ atmosphere. LCMS showed the starting material was consumed completely. The crude product was purified by reversed-phase HPLC (column: Waters Xbridge Prep OBD C18 150*40 mm*10 um; mobile phase: [H₂O (10 mM NH₄HCO₃)-ACN]; gradient: 25%-55% B over 8.0 min) to provide product (28.8 mg, 48.5 μmol, 42% yield) as a white solid.

LCMS: RT=1.417 min, MS cal.: 593.1, [M+H]⁺=593.1

HPLC: RT=10.532 min, purity=99.85%

¹H NMR (400 MHz, METHANOL-d₄) δ=8.60 (d, J=2.4 Hz, 1H), 7.90-7.85 (m, 2H), 7.65 (d, J=8.4 Hz, 1H), 7.45 (s, 1H), 6.84-6.67 (m, 3H), 4.37-4.35 (m, 2H), 3.97 (s, 3H), 3.93 (s, 2H), 3.88-3.86 (m, 2H), 3.45 (s, 3H), 3.09-3.05 (m, 2H), 2.80-2.69 (m, 1H), 2.44-2.30 (m, 2H), 2.01 (s, 3H), 1.92-1.80 (m, 4H)

### Example A17. —Alternative preparation of compound 72. 2-((4-((S)-2-(5-Chloropyridin-2-yl)-2-methylbenzo[d][1,3]dioxol-4-yl)piperidin-1-yl)methyl)-1-(((S)-oxetan-2-yl)methyl)-4-(oxetan-3-yloxy)-1H-benzo[d]imidazole-6-carboxylic acid (Compound 72) (8)

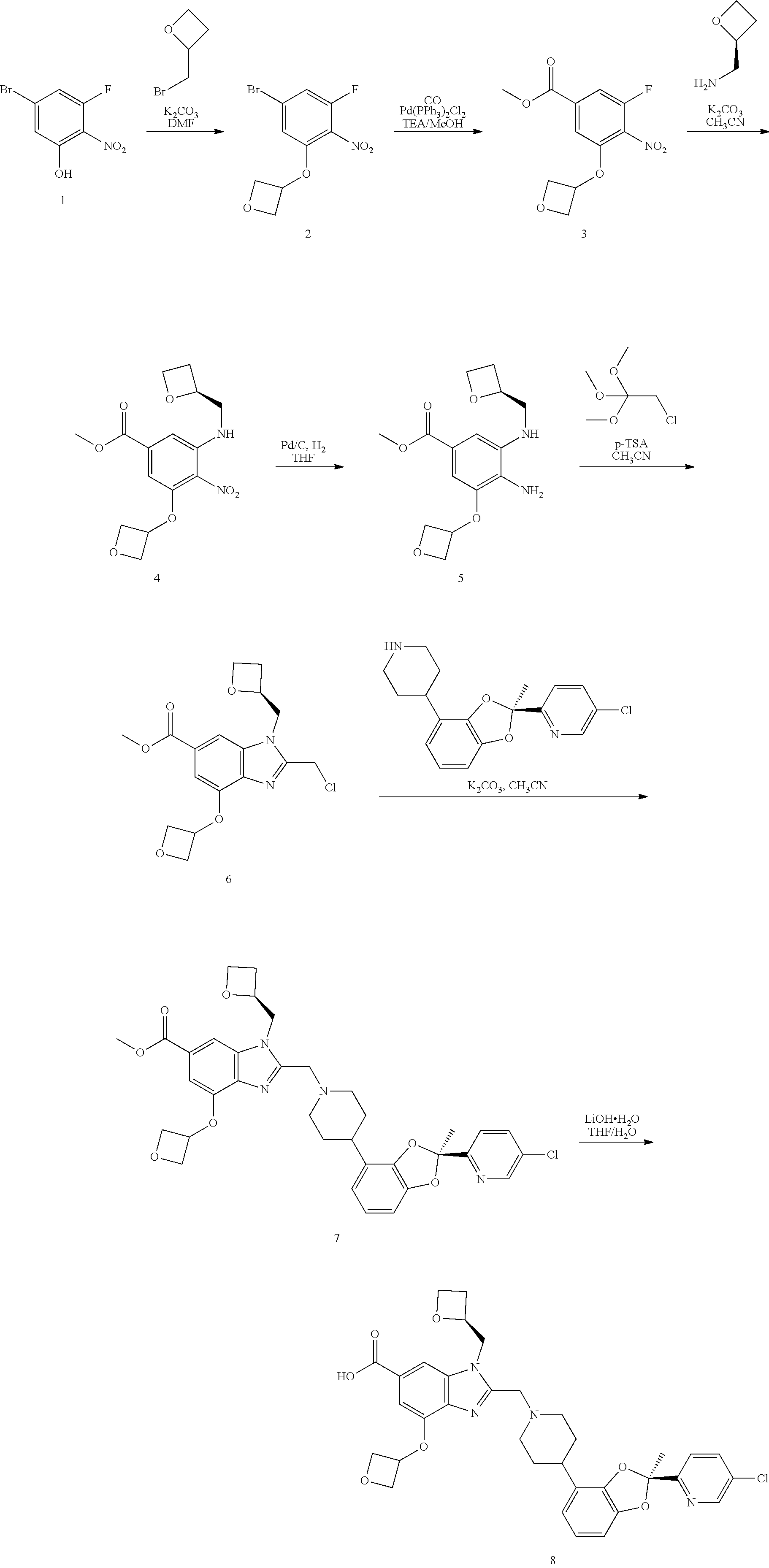

### Preparation of Intermediate 3-(5-Bromo-3-fluoro-2-nitrophenoxy) oxetane (2)

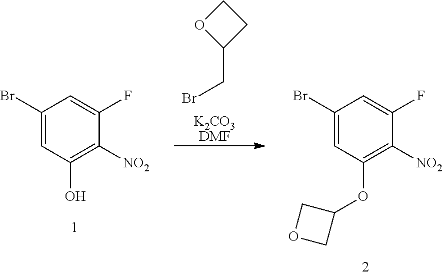

To a solution of 5-bromo-3-fluoro-2-nitrophenol 1 (3 g, 12.7 mmol, 1 eq) in DMF (30 mL) was added K₂CO₃ (1.93 g, 14 mmol, 1.1 eq) and 3-(bromomethyl) oxetane (2.88 g, 19.07 mmol, 1.5 eq). The mixture was stirred at 50° C. for 12 h. LCMS showed starting material was consumed. 50 mL of H₂O was added to the reaction mixture and extracted with EtOAc (50 mL*3) and the combined organic phase was concentrated in vacuum. The residue was purified by column chromatography (SiO₂, PE/EtOAc=10/1 to 1/1) to provide product (3.4 g, 11.1 mmol, 87% yield) as a brown solid.

LCMS: RT=0.479 min, MS cal.: 305.0/307.0, [M+H]⁺=306.0/308.

### Preparation of Intermediate Methyl 3-fluoro-4-nitro-5-(oxetan-3-yloxy)benzoate (3)

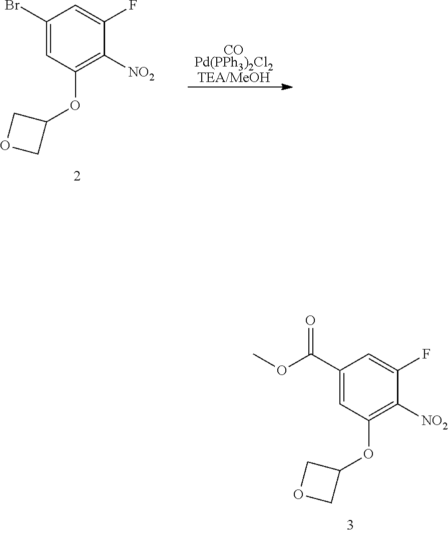 [SMILES: O=[N+]([O-])c1c(F)cc(Br)cc1OC1COC1 ; "COPd(PPh3)2Cl2TEA/MeOH" ; COC(=O)c1cc(F)c([N+](=O)[O-])c(OC2COC2)c1]

MeOH (25 mL) and TEA (12 mL) was charged to the 75 mL hydrogenation bottle, then 3-(5-bromo-3-fluoro-2-nitrophenoxy) oxetane 2 (3.3 g, 10.78 mmol, 1 eq) and Pd(PPh₃)₂Cl₂ (757 mg, 1.08 mmol, 0.1 eq) was added at 20° C. within 0.5 h. After the addition, the mixture was degassed and purged with CO for 3 times. The mixture was stirred at 80° C. (50 Psi) for 23.5 h. TLC (PE/EtOAc=2/1) indicated starting material was consumed completely. The reaction mixture was diluted with H₂O (50 mL) and adjusted pH to 7-8, which was extracted with EtOAc (20 mL*3). The combined organic layers were washed with brine (50 ml*2), dried over Na₂SO₄, filtered, and concentrated under reduced pressure to give a crude product. The crude product was triturated with PE (20 mL) at 20° C. for 20 min to provide product (2.6 g, 9.12 mmol, 85% yield) as a yellow solid.

LCMS: RT=0.440 min, MS cal.: 285.06, [M+H]⁺=286.2

¹H NMR (400 MHz, CHLOROFORM-d)>=7.58-7.52 (m, 2H), 4.93-4.84 (m, 2H), 4.54-4.48 (m, 2H), 4.42 (d, J=7.2 Hz, 2H), 3.98 (s, 3H), 3.51-3.41 (m, 1H)

### Preparation of Intermediate Methyl (S)-4-nitro-3-((oxetan-2-ylmethyl)amino)-5-(oxetan-3-yloxy)benzoate (4)

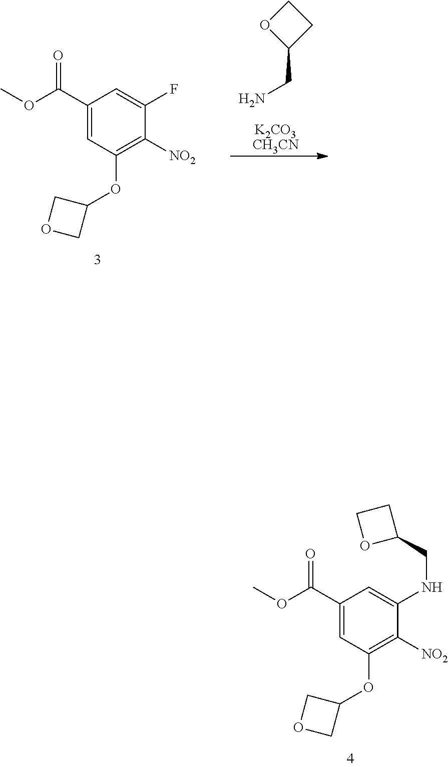

To a solution of methyl 3-fluoro-4-nitro-5-(oxetan-3-yloxy)benzoate 3 (700 mg, 2.45 mmol, 1 eq) in CH₃CN (7 mL) was added K₂CO₃ (1.02 g, 7.36 mmol, 3 eq) and(S)-oxetan-2-ylmethanamine (955 mg, 3.68 mmol, 1.5 eq, p-TSA). The mixture was stirred at 60° C. for 4 h. LC-MS showed starting material was consumed completely and one main peak with desired mass was detected. The mixture was diluted with H₂O (7 mL), extracted with EtOAc (7 mL*3). The combined organic layer was washed with 7 mL H₂O, 7 mL brine, dried over Na₂SO₄, filtered and the filtrate was concentrated to give a crude product. The residue was purified by column chromatography (SiO₂, PE/EtOAc=10/1 to 0/1) to provide product (470 mg, 1.33 mmol, 54% yield) as a white solid.

LCMS: RT=0.408 min, MS cal.: 352.13, [M+H]⁺=353.0

¹H NMR (400 MHz, CHLOROFORM-d) δ=7.17 (d, J=1.2 Hz, 1H), 6.98 (d, J=1.2 Hz, 1H), 6.19-6.12 (m, 1H), 5.13-5.05 (m, 1H), 4.91-4.85 (m, 2H), 4.77-4.68 (m, 1H), 4.62-4.52 (m, 3H), 4.36 (d, J=7.2 Hz, 2H), 3.95 (s, 3H), 3.54-3.42 (m, 3H), 2.78-2.67 (m, 1H), 2.61-2.52 (m, 1H)

### Preparation of Intermediate Methyl (S)-4-amino-3-((oxetan-2-ylmethyl)amino)-5-(oxetan-3-yloxy)benzoate (5)

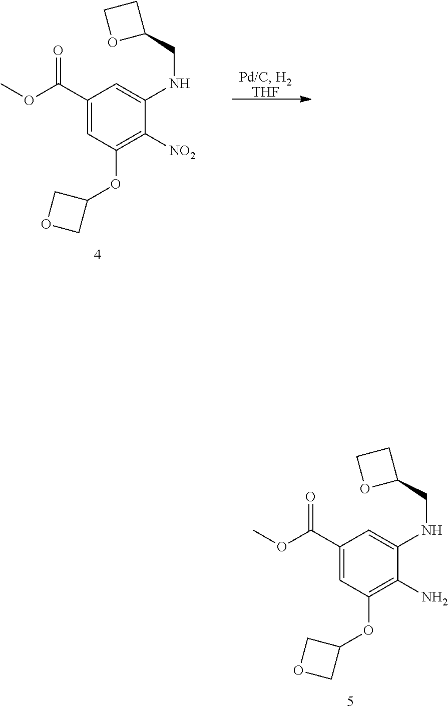 [SMILES: COC(=O)c1cc(NC[C@@H]2CCO2)c([N+](=O)[O-])c(OC2COC2)c1 ; "Pd/C, H2THF" ; COC(=O)c1cc(NC[C@@H]2CCO2)c(N)c(OC2COC2)c1]

To a solution of methyl (S)-4-nitro-3-((oxetan-2-ylmethyl)amino)-5-(oxetan-3-yloxy)benzoate 4 (660 mg, 1.87 mmol, 1 eq) in THF (7 mL) was added Pd/C (660 mg, 10%) under N₂. The suspension was degassed and purged with H₂ for 3 times. The mixture was stirred under H₂ (15 psi) at 20° C. for 1 h. LC-MS showed starting material was consumed completely and one main peak with desired mass was detected. The suspension was filtered and the filter cake was washed with THF (5 mL×3). The combined filtrates were concentrated to dryness to provide product (800 mg, crude) as a yellow oil.

LCMS: RT=0.331 min, MS cal.: 322.15, [M+H]⁺=323.2

¹H NMR (400 MHz, CHLOROFORM-d) δ=7.20 (d, J=1.6 Hz, 1H), 7.17 (d, J=1.2 Hz, 1H), 5.14-5.06 (m, 1H), 4.93-4.88 (m, 2H), 4.78-4.71 (m, 1H), 4.65-4.58 (m, 3H), 4.32-4.28 (m, 2H), 3.88 (s, 3H), 3.51-3.42 (m, 2H), 3.39 (d, J=4.0 Hz, 1H), 2.80-2.70 (m, 1H), 2.64-2.53 (m, 1H)

### Preparation of Intermediate Methyl (S)-2-(chloromethyl)-1-(oxetan-2-ylmethyl)-4-(oxetan-3-yloxy)-1H-benzo[d]imidazole-6-carboxylate (6)

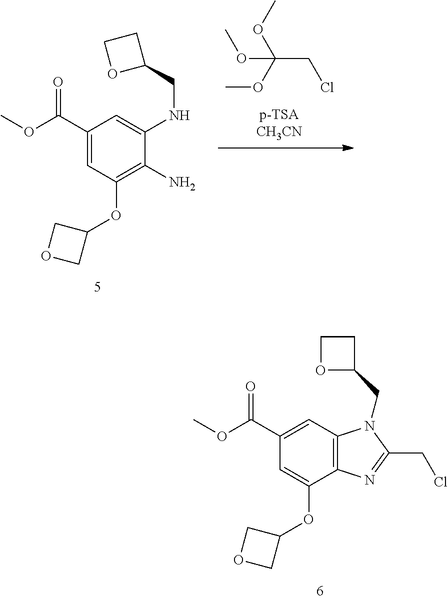

To a solution of methyl (S)-4-amino-3-((oxetan-2-ylmethyl)amino)-5-(oxetan-3-yloxy)benzoate 5 (350 mg, 1.09 mmol, 1 eq) in CH₃CN (5 mL) was added 2-chloro-1,1,1-trimethoxyethane (336 mg, 2.17 mmol, 293 μL, 2 eq) and p-TSA (37.4 mg, 217 μmol, 0.2 eq). The mixture was stirred at 60° C. for 4 hr. LC-MS showed starting material was consumed completely and one main peak with desired mass was detected. The mixture was diluted with H₂O (5 mL), extracted with EtOAc (5 mL*3). The combined organic layer was washed with 5 mL H₂O, 5 mL brine, dried over Na₂SO₄, filtered and the filtrate was concentrated to give a crude product. The residue was purified by column chromatography (SiO₂, PE/EtOAc=10/1 to 0/1) to provide product (230 mg, 604 μmol, 56% yield) as a yellow solid.

LCMS: RT=0.373 min, MS cal.: 380.1/381.1, [M+H]⁺=381.1/383.0

¹H NMR (400 MHz, CHLOROFORM-d) δ=7.78 (s, 1H), 7.48-7.44 (s, 1H), 5.25-5.18 (m, 1H), 5.11-5.01 (m, 2H), 4.99-4.93 (m, 2H), 4.64-4.54 (m, 7H), 4.35-4.28 (m, 1H), 3.97 (s, 3H), 3.70-3.58 (m, 1H), 2.80-2.69 (m, 1H), 2.47-2.34 (m, 1H)

### Preparation of Intermediate Methyl 2-((4-((S)-2-(5-chloropyridin-2-yl)-2-methylbenzo[d][1,3]dioxol-4-yl)piperidin-1-yl)methyl)-1-(((S)-oxetan-2-yl)methyl)-4-(oxetan-3-yloxy)-1H-benzo[d]imidazole-6-carboxylate (7)

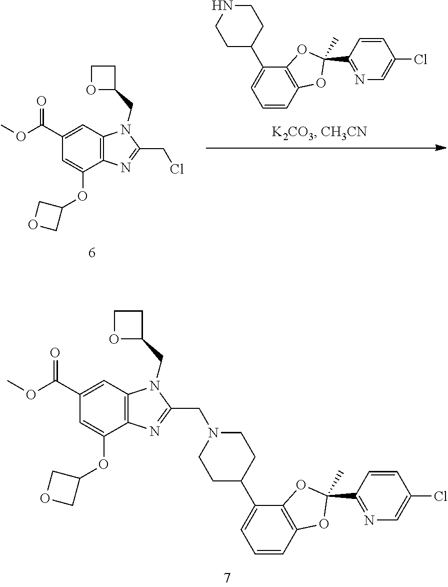 [SMILES: COC(=O)c1cc(OC2COC2)c2nc(CCl)n(C[C@@H]3CCO3)c2c1 ; C[C@]1(c2ccc(Cl)cn2)Oc2cccc(C3CCNCC3)c2O1 ; COC(=O)c1cc(OC2COC2)c2nc(CN3CCC(c4cccc5c4O[C@@](C)(c4ccc(Cl)cn4)O5)CC3)n(C[C@@H]3CCO3)c2c1]

To a solution of methyl (S)-2-(chloromethyl)-1-(oxetan-2-ylmethyl)-4-(oxetan-3-yloxy)-1H-benzo[d]imidazole-6-carboxylate 6 (200 mg, 525 μmol, 1 eq) in CH₃CN (4 mL) was added K₂CO₃ (218 mg, 1.58 mmol, 3 eq) and(S)-5-chloro-2-(2-methyl-4-(piperidin-4-yl)benzo[d][1,3]dioxol-2-yl)pyridine (208 mg, 630 μmol, 1.2 eq). The mixture was stirred at 60° C. for 4 h. LC-MS showed starting material was consumed completely and one main peak with desired mass was detected. The mixture was diluted with H₂O (2 mL), extracted with EtOAc (2 mL*3). The combined organic layer was washed with 2 mL H₂O, 2 mL brine, dried over Na₂SO₄, filtered and the filtrate was concentrated to provide product (265 mg, 392 μmol, 75% yield) as a yellow solid.

LCMS: RT=0.441 min, MS cal.: 674.3/675.3, [M+H]⁺=675.4/676.3.

### Preparation of 2-((4-((S)-2-(5-Chloropyridin-2-yl)-2-methylbenzo[d][1,3]dioxol-4-yl)piperidin-1-yl)methyl)-1-(((S)-oxetan-2-yl)methyl)-4-(oxetan-3-yloxy)-1H-benzo[d]imidazole-6-carboxylic acid Compound 72 (8)

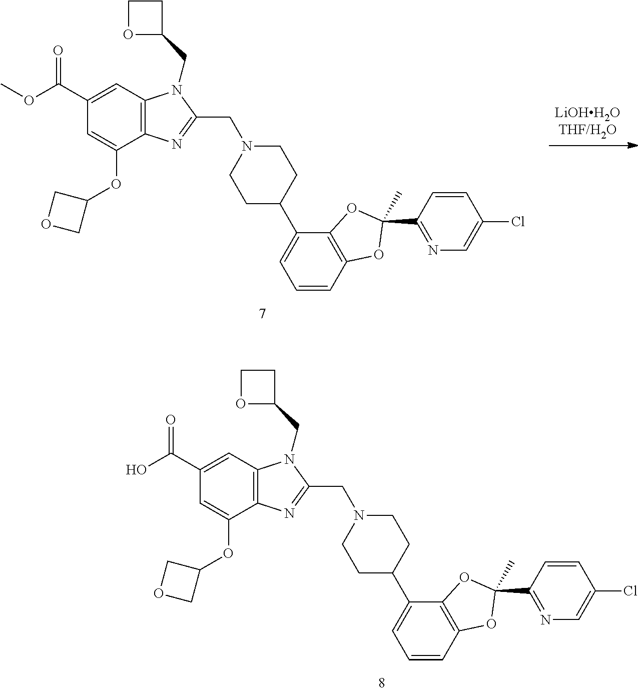 [SMILES: COC(=O)c1cc(OC2COC2)c2nc(CN3CCC(c4cccc5c4O[C@@](C)(c4ccc(Cl)cn4)O5)CC3)n(C[C@@H]3CCO3)c2c1 ; C[C@]1(c2ccc(Cl)cn2)Oc2cccc(C3CCN(Cc4nc5c(OC6COC6)cc(C(=O)O)cc5n4C[C@@H]4CCO4)CC3)c2O1]

To a solution of methyl 2-((4-((S)-2-(5-chloropyridin-2-yl)-2-methylbenzo[d][1,3]dioxol-4-yl)piperidin-1-yl)methyl)-1-(((S)-oxetan-2-yl)methyl)-4-(oxetan-3-yloxy)-1H-benzo[d]imidazole-6-carboxylate 7 (100 mg, 148 μmol, 1 eq) in THF (1 mL) was added LiOH·H₂O (10.6 mg, 252 μmol, 1.7 eq) in H₂O (1 mL). The mixture was stirred at 20° C. for 18 h. LC-MS showed starting material was consumed completely and one main peak with desired mass was detected. The reaction mixture was filtered directly. The residue was purified by prep-HPLC (column: Waters Xbridge Prep OBD C18 150*40 mm*10 um; mobile phase: [H₂O (10 mM NH₄HCO₃)-ACN]; gradient: 35%-65% B over 8.0 min) to provide product (24.8 mg, 37.8 μmol, 25% yield) as a white solid.

LCMS: RT=0.409 min, MS cal.: 660.2/661.2, [M+H]⁺=661.3/663.3

QC:

LCMS: RT=2.610 min, MS cal.: 660.2/661.2, [M+H]⁺=661.3/662.3

HPLC: RT=10.259 min, purity: 100%

¹H NMR (400 MHz, DMSO-d₆) δ=8.71 (d, J=2.0 Hz, 1H), 8.03 (dd, J=2.4, 8.4 Hz, 1H), 7.90 (s, 1H), 7.60 (d, J=8.8 Hz, 1H), 7.30 (s, 1H), 6.82-6.73 (m, 3H), 5.12-5.04 (m, 1H), 4.78-4.70 (m, 3H), 4.65-4.57 (m, 1H), 4.52-4.43 (m, 5H), 4.38-4.30 (m, 1H), 3.90 (d, J=13.2 Hz, 1H), 3.76 (d, J=13.2 Hz, 1H), 3.52-3.44 (m, 1H), 3.01-2.94 (m, 1H), 2.85-2.79 (m, 1H), 2.67-2.59 (m, 2H), 2.44-2.37 (m, 1H), 2.26-2.10 (m, 2H), 2.00 (s, 3H), 1.82-1.65 (m, 4H)

### Example A18. —Preparation of Compound 78. 2-((4-((S)-2-(5-Chloropyridin-2-yl)-2-methylbenzo[d][1,3]dioxol-4-yl)piperidin-1-yl)methyl)-4-(difluoromethoxy)-1-(((S)-oxetan-2-yl)methyl)-1H-benzo[d]imidazole-6-carboxylic acid (compound 78) (11)

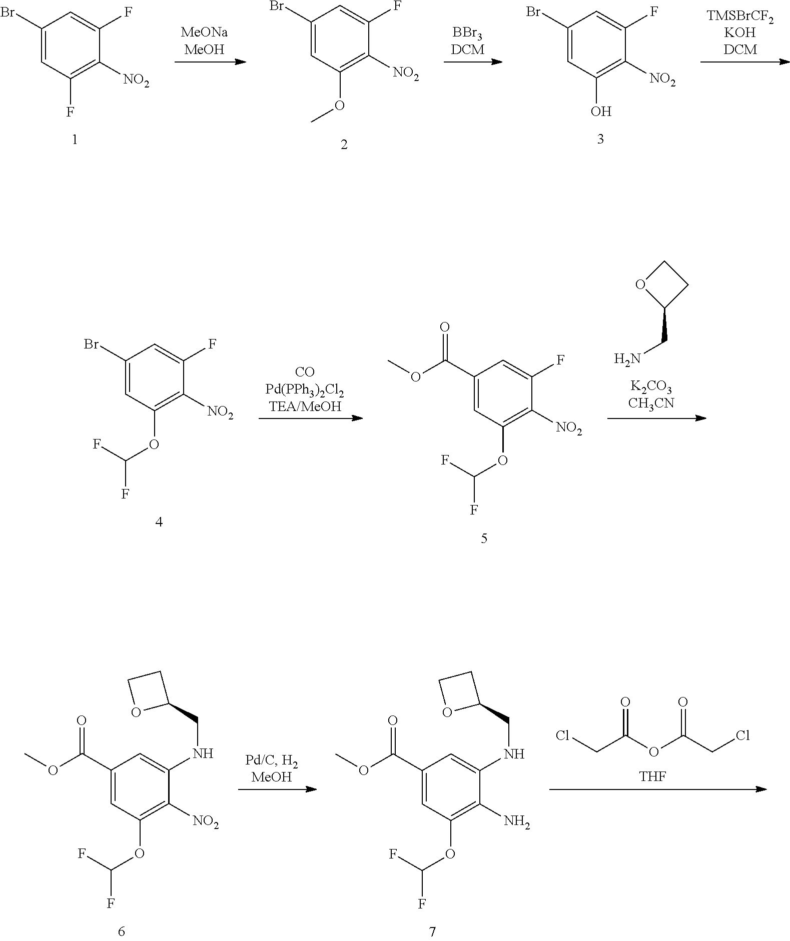

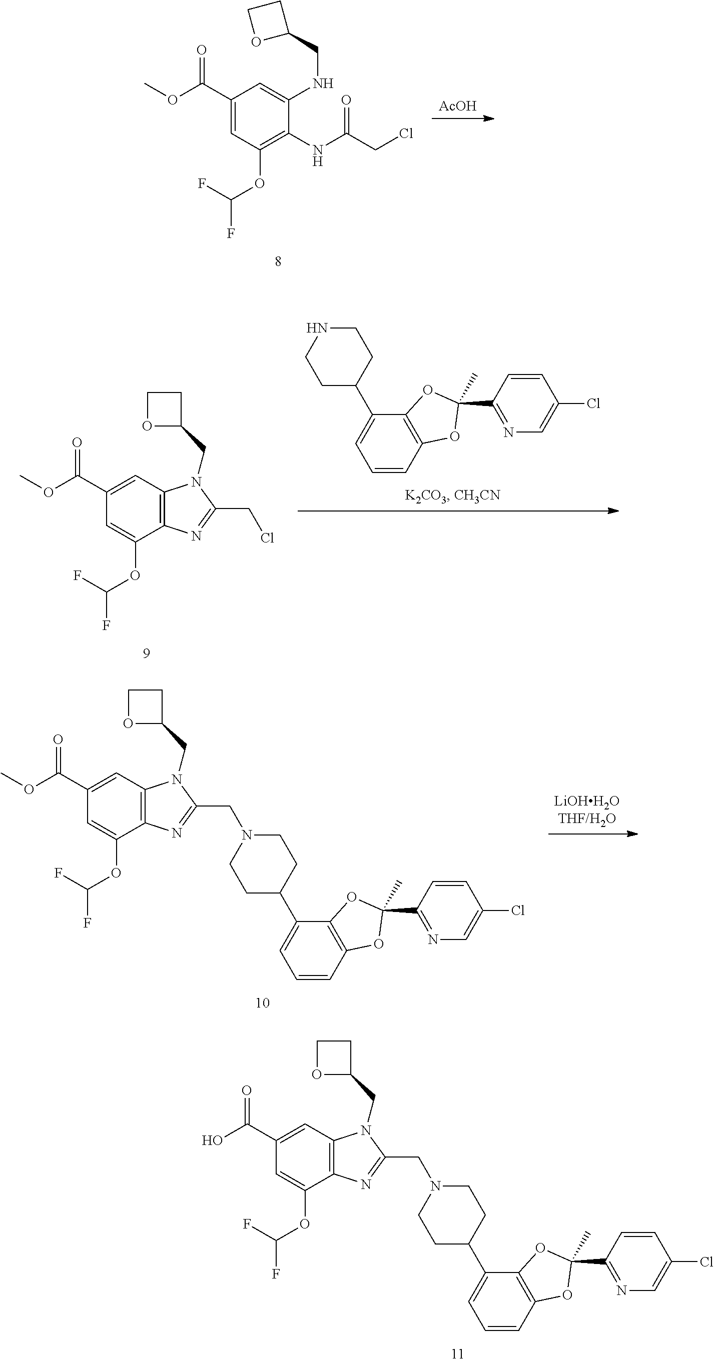 [SMILES: "AcOH" ; COC(=O)c1cc(NC[C@@H]2CCO2)c(NC(=O)CCl)c(OC(F)F)c1 ; COC(=O)c1cc(OC(F)F)c2nc(CCl)n(C[C@@H]3CCO3)c2c1 ; C[C@]1(c2ccc(Cl)cn2)Oc2cccc(C3CCNCC3)c2O1 ; COC(=O)c1cc(OC(F)F)c2nc(CN3CCC(c4cccc5c4O[C@@](C)(c4ccc(Cl)cn4)O5)CC3)n(C[C@@H]3CCO3)c2c1 ; C[C@]1(c2ccc(Cl)cn2)Oc2cccc(C3CCN(Cc4nc5c(OC(F)F)cc(C(=O)O)cc5n4C[C@@H]4CCO4)CC3)c2O1]

### Preparation of Intermediate 5-Bromo-1-fluoro-3-methoxy-2-nitrobenzene (2)

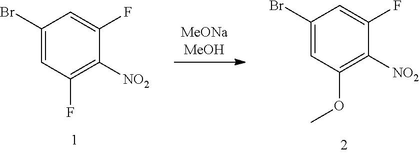

To a solution of 5-bromo-1,3-difluoro-2-nitrobenzene 1 (5 g, 21.0 mmol, 1 eq) in MeOH (50 mL) was added MeONa (3.41 g, 63.0 mmol, 3 eq). The mixture was stirred at 70° C. for 12 h. TLC indicated starting material was consumed completely and one new spot formed. The reaction was clean according to TLC. The reaction mixture was concentrated under reduced pressure to remove MeOH to give a residue. The residue was diluted by EtOAc (30 mL). The organic phase was washed by H₂O (10 mL), aq. Na₂CO₃ (10 mL), dried with Na₂SO₄, filtered. The organic phase was concentrated under reduced pressure at 40° C. to provide product (5.2 g, 20.8 mmol, 98% yield) as a white solid.

¹H NMR (400 MHz, DMSO-d₆) δ=7.57 (dd, J=2.0, 9.2 Hz, 1H), 7.51 (t, J=1.6 Hz, 1H), 3.97 (s, 3H)

### Preparation of Intermediate 5-Bromo-3-fluoro-2-nitrophenol (3)

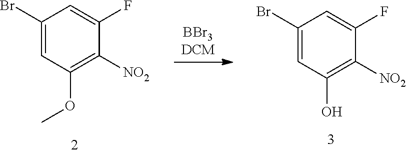

DCM (50 mL) was charged to the three-necked round bottom flask, then 5-bromo-1-fluoro-3-methoxy-2-nitrobenzene 2 (5 g, 20.0 mmol, 1 eq) was added to the mixture at 20° C. At −40° C. (inner temperature), BBr₃ (1 M, 26.0 mL, 1.3 eq) was added to the reaction mixture at −40° C. within 10 min. After the addition, the mixture was stirred at 20° C. for 15 h. LC-MS showed starting material consumed and one main peak with desired mass was detected. TLC indicated starting material was consumed completely and one new spot formed. The reaction was clean according to TLC. The reaction mixture was quenched by addition H₂O (50 mL) at 0° C., and extracted with DCM 60 mL (30 mL*2), and the combined organic layers were washed sequentially with 5% aqueous NaHCO₃ (20 mL) and brine (20 mL), dried over Na₂SO₄, filtered and concentrated under reduced pressure to provide product (4.5 g, 19.1 mmol, 95% yield) as a white solid.

LCMS: RT=0.568 min, MS cal.: 234.9, 236.9, [M+H]⁺=233.9

¹H NMR (400 MHz, DMSO-d₆) δ=12.13 (br s, 1H), 7.34 (dd, J=2.0, 9.6 Hz, 1H), 7.10 (t, J=1.6 Hz, 1H)

### Preparation of Intermediate 5-Bromo-1-(difluoromethoxy)-3-fluoro-2-nitrobenzene (4)

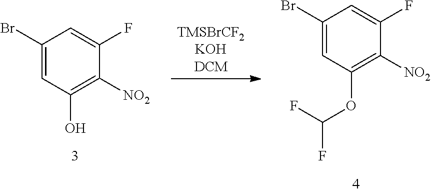

To a solution of 5-bromo-3-fluoro-2-nitrophenol 3 (3 g, 12.7 mmol, 1 eq) in DCM (30 mL) was added dropwise aq. KOH (21.4 g, 76.3 mmol, 20% purity, 6 eq) at 0° C., then TMSBrCF₂ (5.16 g, 25.4 mmol, 2 eq) in DCM (5 mL) was added dropwise at 0° C. The mixture was stirred at 20° C. for 1 h. TLC indicated starting material was consumed completely, the reaction was clean according to TLC. One major new spot with lower polarity was detected (R_{f}=0.7). The reaction mixture was diluted with H₂O (50 mL) and extracted with EtOAc 300 mL (100 mL*3). The combined organic layers were washed with brine (60 mL), dried with Na₂SO₄, filtered and concentrated under reduced pressure to provide product (3.4 g, 11.9 mmol, 93% yield) as an orange oil.

¹H NMR (400 MHz, DMSO-d₆) δ=7.98 (dd, J=1.6, 9.6 Hz, 1H), 7.76 (s, 1H), 7.49 (t, J=72.4 Hz, 1H)

### Preparation of Intermediate Methyl 3-(difluoromethoxy)-5-fluoro-4-nitrobenzoate (5)

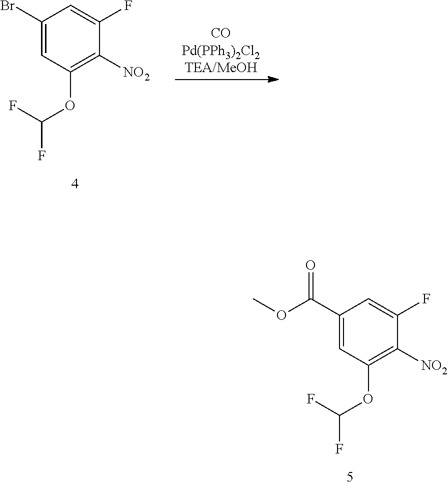 [SMILES: O=[N+]([O-])c1c(F)cc(Br)cc1OC(F)F ; "COPd(PPh3)2Cl2TEA/MeOH" ; COC(=O)c1cc(F)c([N+](=O)[O-])c(OC(F)F)c1]

To a solution of 5-bromo-1-(difluoromethoxy)-3-fluoro-2-nitrobenzene 4 (2 g, 6.99 mmol, 1 eq) in MeOH (20 mL) and TEA (10 mL) was added Pd(PPh₃)₂Cl₂ (491 mg, 699 μmol, 0.1 eq) under N₂ atmosphere. The suspension was degassed and purged with CO for 3 times. The mixture was stirred under CO (50 psi) at 80° C. for 1 h. TLC indicated ˜30% starting material remained, and one new spot with larger polarity was detected. The reaction was clean according to TLC (R_{f}=0.56). The reaction mixture was filtered, and the filtrate was diluted with H₂O (50 mL) and extracted with EtOAc 150 mL (50 mL*3). The combined organic layers were washed with brine (50 mL), dried over [Na₂SO₄], filtered and concentrated under reduced pressure to give a residue. The residue was purified by column chromatography (SiO₂, Petroleum ether/Ethyl acetate=1/0 to 5/1) to provide product (1.10 g, 4.15 mmol, 59% yield) as an orange oil

¹H NMR (400 MHz, DMSO-d6) δ=8.01 (dd, J=1.2, 9.6 Hz, 1H), 7.87 (s, 1H), 7.60 (t, J=72.4 Hz, 1H), 3.93 (s, 3H)

### Preparation of Intermediate Methyl (S)-3-(difluoromethoxy)-4-nitro-5-((oxetan-2-ylmethyl)amino)benzoate (6)

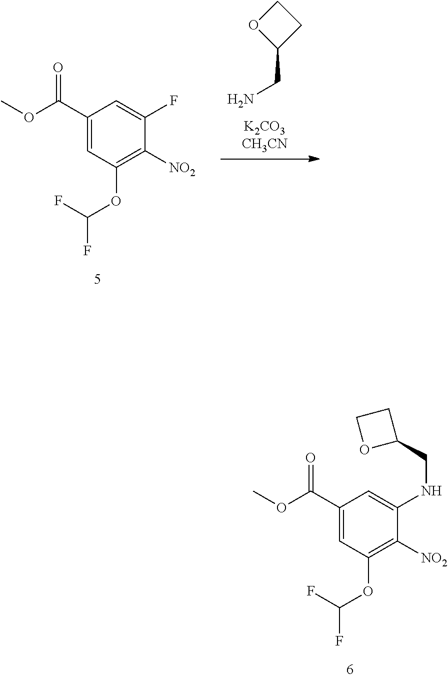

To a solution of methyl 3-(difluoromethoxy)-5-fluoro-4-nitrobenzoate 5 (500 mg, 1.89 mmol, 1 eq) and(S)-oxetan-2-ylmethanamine (734 mg, 2.83 mmol, 1.5 eq, p-TSA) in ACN (10 mL) and DMF (2 mL) was added K₂CO₃ (782 mg, 5.66 mmol, 3 eq). The mixture was stirred at 60° C. for 4 h. LC-MS showed starting material was consumed completely and one main peak with desired mass was detected. The reaction mixture was diluted with H₂O (30 mL) and extracted with EtOAc 90 mL (30 mL*3). The combined organic layers were washed with brine (20 mL), dried with Na₂SO₄, filtered and concentrated under reduced pressure to give a residue. The residue was purified by column chromatography (SiO₂, Petroleum ether/Ethyl acetate=10/1 to 0/1) to provide product (500 mg, 1.50 mmol, 79% yield) as an orange oil.

LCMS: RT=0.805 min, MS cal.: 332.1, [M−H]⁻=331.2

¹H NMR (400 MHz, ACETONITRILE-d3) δ=7.52 (s, 1H), 7.07 (s, 1H), 6.83 (t, J=72.8 Hz, 1H), 6.21 (br s, 1H), 5.03-4.92 (m, 1H), 4.61-4.59 (m, 1H), 4.48-4.44 (m, 1H), 3.89 (s, 3H), 3.58-3.46 (m, 2H), 2.73-2.63 (m, 1H), 2.53-2.50 (tm, 1H)

### Preparation of Intermediate Methyl (S)-4-amino-3-(difluoromethoxy)-5-((oxetan-2-ylmethyl)amino)benzoate (7)

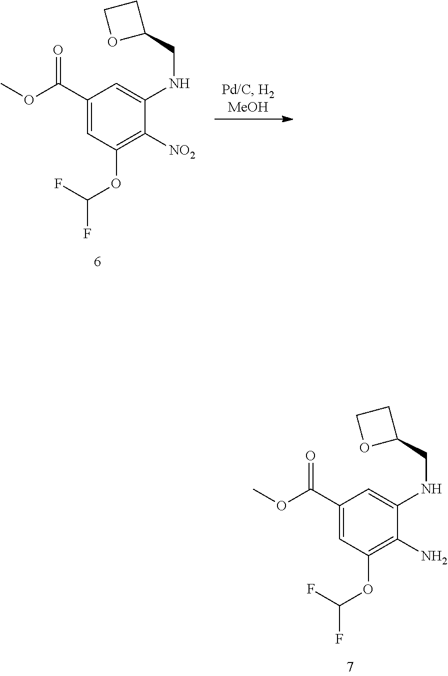 [SMILES: COC(=O)c1cc(NC[C@@H]2CCO2)c([N+](=O)[O-])c(OC(F)F)c1 ; "Pd/C, H2MeOH" ; COC(=O)c1cc(NC[C@@H]2CCO2)c(N)c(OC(F)F)c1]

To a solution of Pd/C (100 mg, 10%) in MeOH (5 mL) was added methyl (S)-3-(difluoromethoxy)-4-nitro-5-((oxetan-2-ylmethyl)amino)benzoate 6 (500 mg, 1.50 mmol, 1 eq) under N₂ atmosphere. The suspension was degassed and purged with H₂ for 3 times. The mixture was stirred under H₂ (15 psi) at 20° C. for 12 h. LC-MS showed starting material was consumed completely and one main peak with desired mass was detected. After 12 h, the suspension was filtered through a pad of Celite and filter cake was washed with MeOH (20 mL×3). The combined filtrates were concentrated to dryness to give product (440 mg, 1.46 mmol, 96% yield) as a light yellow oil.

¹H NMR (400 MHz, ACETONITRILE-d3) δ=7.23 (s, 1H), 7.21 (s, 1H), 6.71 (t, J=74.4 Hz, 1H), 5.03-4.95 (m, 1H), 4.64-4.62 (m, 1H), 4.53-4.51 (m, 1H), 4.48 (br s, 2H), 3.92 (br s, 1H), 3.80 (s, 3H), 3.45-3.30 (m, 2H), 2.75-2.65 (m, 1H), 2.59-2.49 (m, 1H)

### Preparation of Intermediate Methyl (S)-4-(2-chloroacetamido)-3-(difluoromethoxy)-5-((oxetan-2-ylmethyl)amino)benzoate (8)

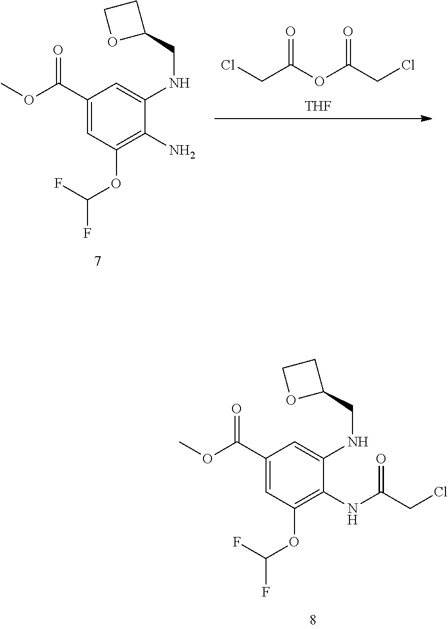 [SMILES: "THF" ; COC(=O)c1cc(NC[C@@H]2CCO2)c(N)c(OC(F)F)c1 ; O=C(CCl)OC(=O)CCl ; COC(=O)c1cc(NC[C@@H]2CCO2)c(NC(=O)CCl)c(OC(F)F)c1]

To a solution of methyl (S)-4-amino-3-(difluoromethoxy)-5-((oxetan-2-ylmethyl)amino)benzoate 7 (200 mg, 662 μmol, 1 eq) in THF (5 mL) was added 2-chloroacetic anhydride (113 mg, 662 μmol, 1 eq). The mixture was stirred at 0° C. for 1 h. LC-MS showed starting material was consumed completely and one main peak with desired mass was detected. The reaction mixture was concentrated under reduced pressure to provide product (250 mg, 660 μmol, 99% yield) as a yellow oil.

LCMS: RT=0.415 min, MS cal.: 378.1, 380.1, [M+H]⁺=379.0

### Preparation of Intermediate Methyl (S)-2-(chloromethyl)-4-(difluoromethoxy)-1-(oxetan-2-ylmethyl)-1H-benzo[d]imidazole-6-carboxylate (9)

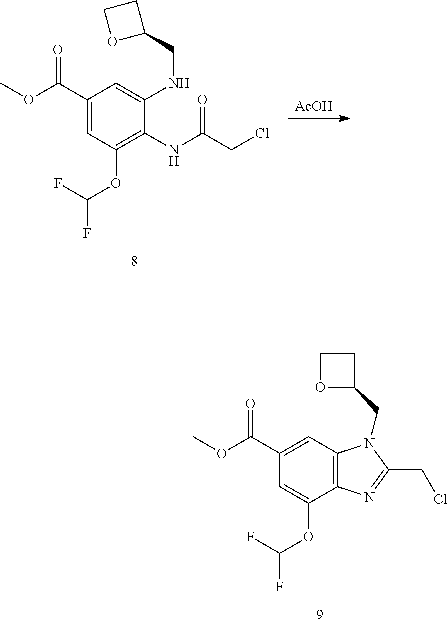 [SMILES: "AcOH" ; COC(=O)c1cc(NC[C@@H]2CCO2)c(NC(=O)CCl)c(OC(F)F)c1 ; COC(=O)c1cc(OC(F)F)c2nc(CCl)n(C[C@@H]3CCO3)c2c1]

A mixture of methyl (S)-4-(2-chloroacetamido)-3-(difluoromethoxy)-5-((oxetan-2-ylmethyl)amino)benzoate 8 (250 mg, 660 μmol, 1 eq) in AcOH (5 mL) was degassed and purged with N₂ for 3 times, and then the mixture was stirred at 80° C. for 12 h under N₂ atmosphere. LC-MS showed starting material was consumed completely and one main peak with desired mass was detected. The reaction mixture was concentrated under reduced pressure to remove solvent to provide product (260 mg, crude) as a yellow oil.

LCMS: RT=0.475 min, MS cal.: 360.1, 362.1, [M+H]⁺=361.0

### Preparation of Intermediate Methyl 2-((4-((S)-2-(5-chloropyridin-2-yl)-2-methylbenzo[d][1,3]dioxol-4-yl)piperidin-1-yl)methyl)-4-(difluoromethoxy)-1-(((S)-oxetan-2-yl)methyl)-1H-benzo[d]imidazole-6-carboxylate (10)

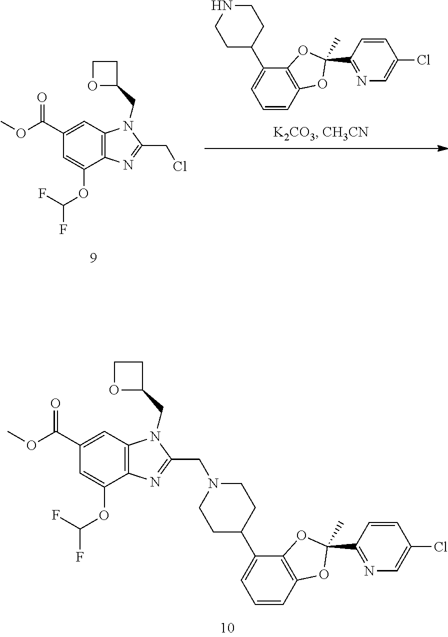 [SMILES: COC(=O)c1cc(OC(F)F)c2nc(CCl)n(C[C@@H]3CCO3)c2c1 ; C[C@]1(c2ccc(Cl)cn2)Oc2cccc(C3CCNCC3)c2O1 ; COC(=O)c1cc(OC(F)F)c2nc(CN3CCC(c4cccc5c4O[C@@](C)(c4ccc(Cl)cn4)O5)CC3)n(C[C@@H]3CCO3)c2c1]

To a solution of methyl (S)-2-(chloromethyl)-4-(difluoromethoxy)-1-(oxetan-2-ylmethyl)-1H-benzo[d]imidazole-6-carboxylate 9 (260 mg, 721 μmol, 1 eq) and(S)-5-chloro-2-(2-methyl-4-(piperidin-4-yl)benzo[d][1,3]dioxol-2-yl)pyridine (358 mg, 1.08 mmol, 1.5 eq) in ACN (10 mL) was added K₂CO₃ (498 mg, 3.60 mmol, 5 eq). The mixture was stirred at 60° C. for 4 h. LC-MS showed starting material was consumed completely and one main peak with desired mass was detected. The reaction mixture was diluted with H₂O (30 mL) and extracted with EtOAc 60 mL (20 mL*3). The combined organic layers were washed with brine (20 mL), dried with Na₂SO₄, filtered and concentrated under reduced pressure to give a residue. The residue was purified by column chromatography (SiO₂, Petroleum ether/Ethyl acetate=95/5 to 3/1) to provide product (300 mg, 458 μmol, 63% yield) as a yellow oil.

LCMS: RT=1.013 min, MS cal.: 654.2, 655.2, [M+H]⁺=655.4

### Preparation of 2-((4-((S)-2-(5-Chloropyridin-2-yl)-2-methylbenzo[d][1,3]dioxol-4-yl)piperidin-1-yl)methyl)-4-(difluoromethoxy)-1-(((S)-oxetan-2-yl)methyl)-1H-benzo[d]imidazole-6-carboxylic acid, Compound 78 (11)

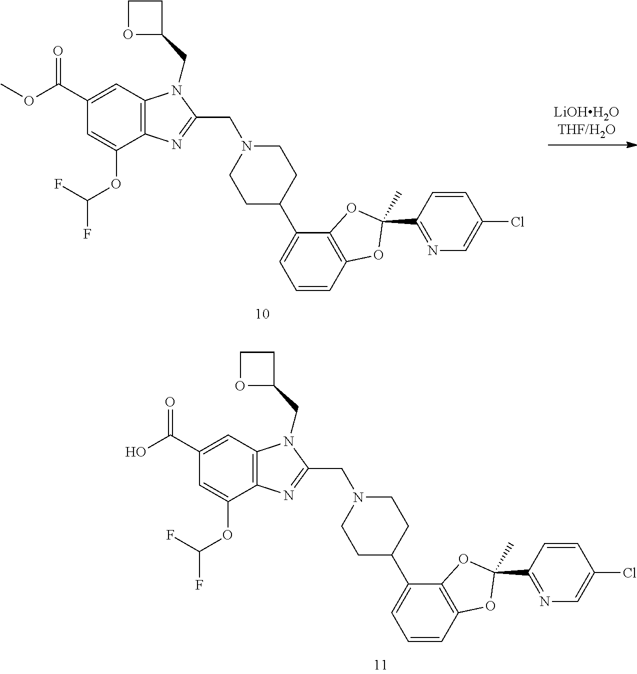 [SMILES: COC(=O)c1cc(OC(F)F)c2nc(CN3CCC(c4cccc5c4O[C@@](C)(c4ccc(Cl)cn4)O5)CC3)n(C[C@@H]3CCO3)c2c1 ; C[C@]1(c2ccc(Cl)cn2)Oc2cccc(C3CCN(Cc4nc5c(OC(F)F)cc(C(=O)O)cc5n4C[C@@H]4CCO4)CC3)c2O1]

A mixture of methyl 2-((4-((S)-2-(5-chloropyridin-2-yl)-2-methylbenzo[d][1,3]dioxol-4-yl)piperidin-1-yl)methyl)-4-(difluoromethoxy)-1-(((S)-oxetan-2-yl)methyl)-1H-benzo[d]imidazole-6-carboxylate 10 (100 mg, 153 μmol, 1 eq), LiOH·H₂O (9.61 mg, 229 μmol, 1.5 eq) in THF (1 mL) and H₂O (0.43 mL) was degassed and purged with N₂ for 3 times, and then the mixture was stirred at 25° C. for 12 h under N₂ atmosphere. LC-MS showed ˜7% of starting material remained. Several new peaks were shown on LC-MS and ˜79% of desired compound was detected. The reaction mixture was concentrated under reduced pressure to remove solvent and dissolved with ACN (1.5 mL). The residue was purified by prep-HPLC (column: Phenomenex Gemini-NX 80*40 mm*3 um; mobile phase: [H₂O (10 mM NH₄HCO₃)-ACN]; gradient: 20%-50% B over 8.0 min) to provide product (28.6 mg, 44.6 μmol, 29% yield) as a white solid.

LCMS: RT=2.729 min, MS cal.: 640.2, 641.2, [M+H]⁺=641.2

HPLC: RT=11.518 min

¹H NMR (400 MHz, DMSO-d₆) δ=8.72 (d, J=2.0 Hz, 1H), 8.19 (d, J=1.2 Hz, 1H), 8.01 (dd, J=2.4, 8.4 Hz, 1H), 7.62 (t, J=74.4 Hz, 1H), 7.60 (d, J=8.8 Hz, 1H), 7.51 (s, 1H), 6.84-6.74 (m, 3H), 5.13-5.10 (m, 1H), 4.82-4.80 (m, 1H), 4.70-4.63 (m, 1H), 4.51-4.43 (m, 1H), 4.39-4.37 (m, 1H), 3.97 (d, J=13.6 Hz, 1H), 3.79 (d, J=13.6 Hz, 1H), 3.02-3.00 (m, 1H), 2.85-2.83 (m, 1H), 2.71-2.62 (m, 2H), 2.47-2.42 (m, 1H), 2.31-2.14 (m, 2H), 2.02 (s, 3H), 1.82-1.67 (m, 4H)

### Example A19. —Preparation of Compound 79. 2-(((1R,4R)-4-((S)-2-(5-chloropyridin-2-yl)-2-methylbenzo[d][1,3]dioxol-4-yl)cyclohexyl)(methyl)amino)-4-methoxy-1-(((S)-oxetan-2-yl)methyl)-1H-benzo[d]imidazole-6-carboxylic acid (Compound 79) (14)

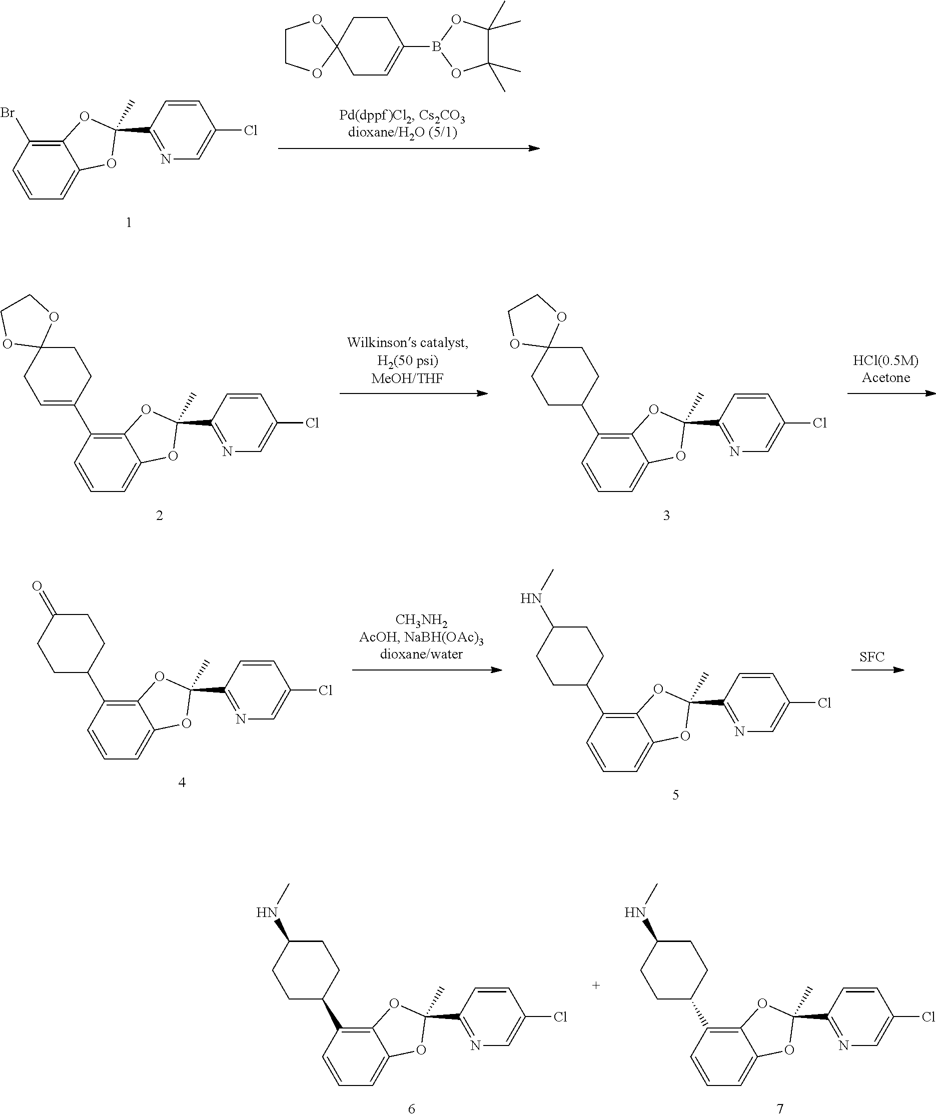

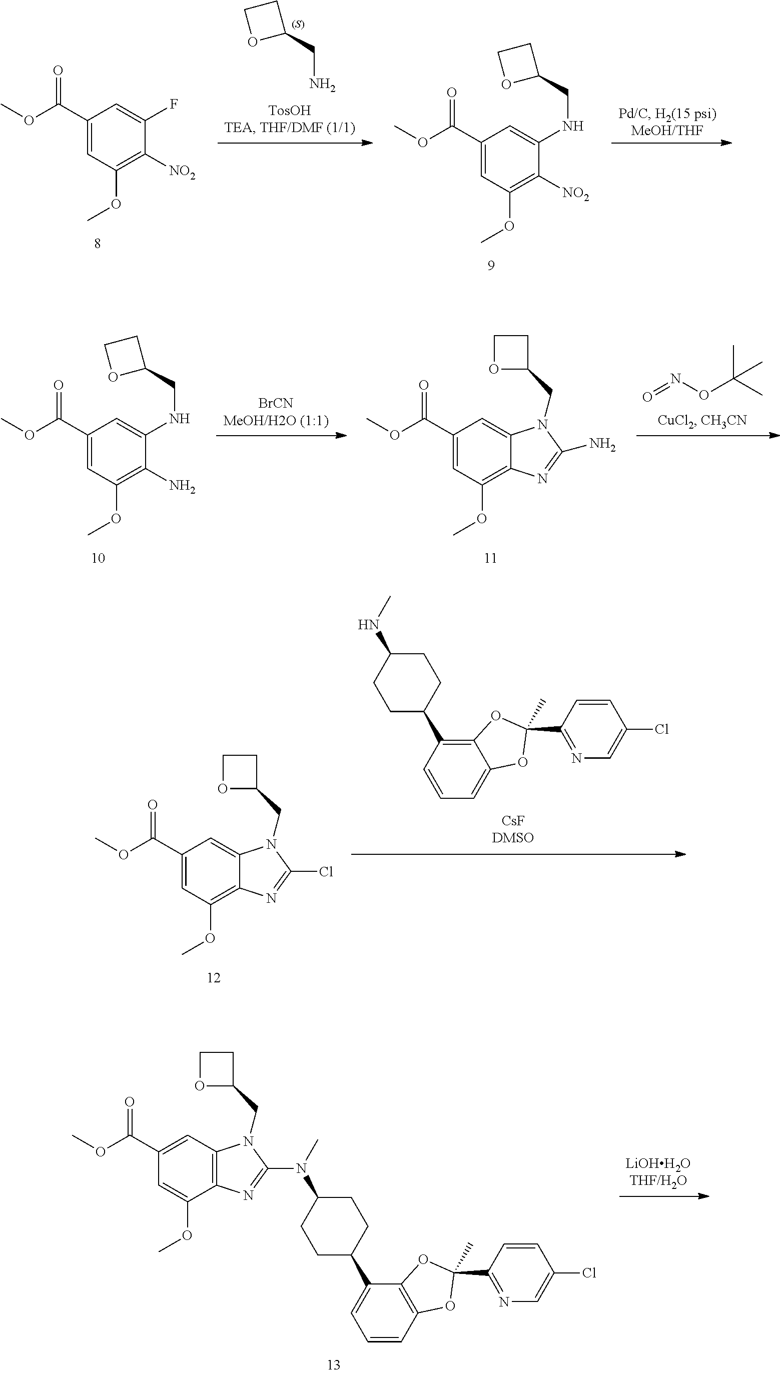

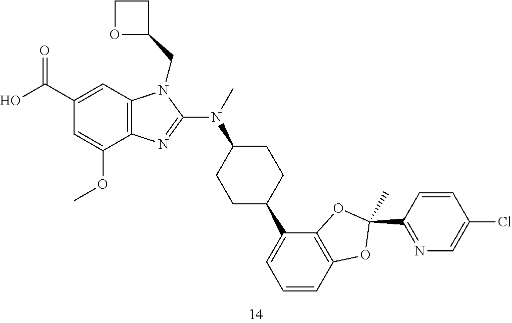 [SMILES: COc1cc(C(=O)O)cc2c1nc(N(C)[C@H]1CC[C@@H](c3cccc4c3O[C@@](C)(c3ccc(Cl)cn3)O4)CC1)n2C[C@@H]1CCO1]

### Preparation of Intermediate (S)-5-Chloro-2-(2-methyl-4-(1,4-dioxaspiro[4.5] dec-7-en-8-yl)benzo[d][1,3]dioxol-2-yl)pyridine (2)

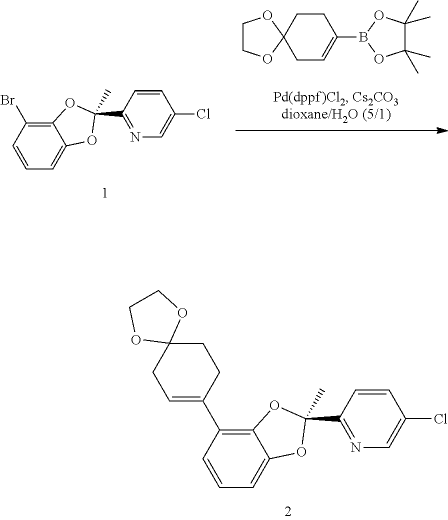 [SMILES: CC1(C)OB(C2=CCC3(CC2)OCCO3)OC1(C)C ; C[C@]1(c2ccc(Cl)cn2)Oc2cccc(Br)c2O1 ; C[C@]1(c2ccc(Cl)cn2)Oc2cccc(C3=CCC4(CC3)OCCO4)c2O1]

A mixture of (S)-2-(4-bromo-2-methylbenzo[d][1,3]dioxol-2-yl)-5-chloropyridine 1 (2.00 g, 6.11 mmol), 4,4,5,5-tetramethyl-2-(1,4-dioxaspiro[4.5] dec-7-en-8-yl)-1,3,2-dioxaborolane (2.44 g, 9.19 mmol, 1.5 eq), Cs₂CO₃ (2.99 g, 9.19 mmol, 1.5 eq), and (Pd(dppf)Cl₂ (448 mg, 612 μmol, 0.1 eq) in dioxane (20 mL) and H₂O (4 mL) was degassed and purged with N₂ for 3 times, and then the mixture was stirred at 90° C. for 12 h under N₂ atmosphere. LCMS showed the starting material was consumed completely. The reaction mixture was quenched by H₂O (10 mL) at 20° C., extracted with ethyl acetate 30 mL (10 mL*3). The combined organic layers were washed with brine 20 mL (10 mL*2), dried over Na₂SO₄, filtered, and concentrated under reduced pressure to give a residue. The residue was purified by column chromatography (SiO₂, Petroleum ether/Ethyl acetate=100/1 to 0/1) to provide product (3 g, crude) was as a white solid.

LCMS: RT=2.363 min, MS cal.: 385.1, [M+H]⁺=386.1

¹H NMR (400 MHz, METHANOL-d4) δ=8.60 (d, J=2.0 Hz, 1H), 7.86 (dd, J=2.4, 8.4 Hz, 1H), 7.62 (d, J=8.4 Hz, 1H), 6.86-6.69 (m, 3H), 6.26-6.18 (m, 1H), 4.01-3.96 (m, 4H), 2.71-2.56 (m, 2H), 2.47-2.38 (m, 2H), 2.02 (s, 3H), 1.88-1.85 (m, 2H)

### Preparation of Intermediate (S)-5-Chloro-2-(2-methyl-4-(1,4-dioxaspiro[4.5]decan-8-yl)benzo[d][1,3]dioxol-2-yl)pyridine (3)

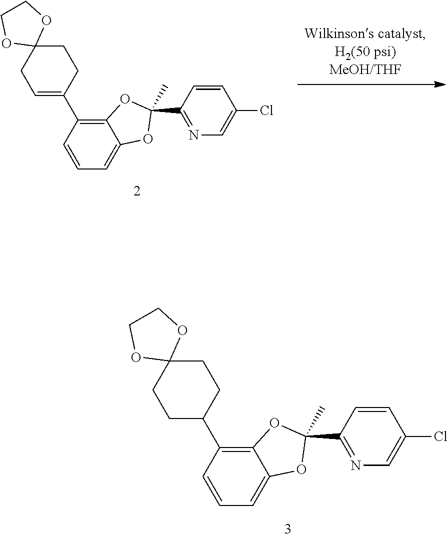 [SMILES: C[C@]1(c2ccc(Cl)cn2)Oc2cccc(C3=CCC4(CC3)OCCO4)c2O1 ; C[C@]1(c2ccc(Cl)cn2)Oc2cccc(C3CCC4(CC3)OCCO4)c2O1]

Equip a hydrogenated bottle (75 mL), MeOH (20 mL) and THF (20 mL) was charged to the hydrogenated bottle, then(S)-5-chloro-2-(2-methyl-4-(1,4-dioxaspiro[4.5] dec-7-en-8-yl)benzo[d][1,3]dioxol-2-yl)pyridine 2 (3 g, 7.78 mmol, 1 eq) and Wilkinson's catalyst (1.50 g) were added to the reaction mixture. After the addition, the suspension was degassed and purged with H₂ for 3 times. Then the reaction mixture was stirred at 60° C. for 24 h under H₂ (50 psi). LCMS showed the starting material was consumed completely. The suspension was filtered and the filter cake was washed with MeOH/THF=1/1 (10 mL*3). The combined filtrates were concentrated to give residue. The residue was purified by column chromatography (SiO₂, Petroleum ether/Ethyl acetate=100/1 to 0/1) to provide product (2.7 g, 6.96 mmol, 90% yield) as a white solid.

LCMS: RT=2.177 min, MS cal.: 387.1, [M+H]⁺=388.3

### Preparation of Intermediate (S)-4-(2-(5-Chloropyridin-2-yl)-2-methylbenzo[d][1,3] dioxol-4-yl)cyclohexan-1-one (4)

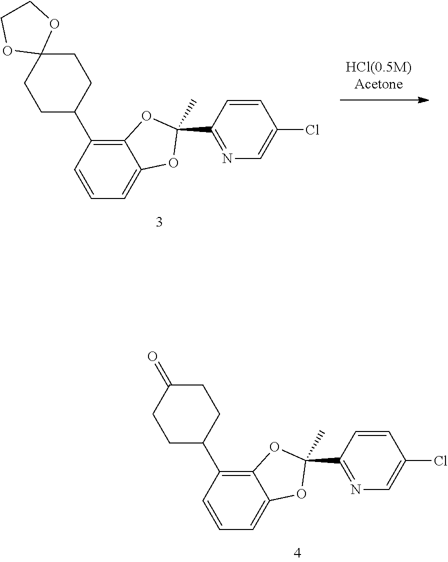 [SMILES: C[C@]1(c2ccc(Cl)cn2)Oc2cccc(C3CCC4(CC3)OCCO4)c2O1 ; C[C@]1(c2ccc(Cl)cn2)Oc2cccc(C3CCC(=O)CC3)c2O1]

A mixture of (S)-5-chloro-2-(2-methyl-4-(1,4-dioxaspiro[4.5]decan-8-yl)benzo[d][1,3]dioxol-2-yl)pyridine 3 (2.7 g, 6.96 mmol, 1 eq) and HCl (0.5 M, 125 mL, 9 eq) in acetone (250 mL) was degassed and purged with N₂ for 3 times, and then the mixture was stirred at 20° C. for 4 h under N₂ atmosphere. LCMS showed the starting material was consumed completely. After 2 hr, the reaction mixture was added to sat. NaHCO₃ to adjust pH=7-8 at 20° C., filtered. The filter cake was dried in vacuum to provide product (2 g, 5.82 mmol, 84% yield) as a white solid.

LCMS: RT=0.558 min, MS cal.: 343.1, [M+H]⁺=344.2

¹H NMR (400 MHz, CHLOROFORM-d) δ=8.64 (d, J=2.0 Hz, 1H), 7.70 (dd, J=2.4, 8.4 Hz, 1H), 7.58 (d, J=8.4 Hz, 1H), 6.85-6.68 (m, 3H), 3.27-3.13 (m, 1H), 2.60-2.43 (m, 4H), 2.31-2.13 (m, 2H), 2.07 (s, 3H), 2.05-1.96 (m, 2H)

### Preparation of Intermediate (S)-4-(2-(5-chloropyridin-2-yl)-2-methylbenzo[d][1,3]dioxol-4-yl)-N-methylcyclohexan-1-amine (5)

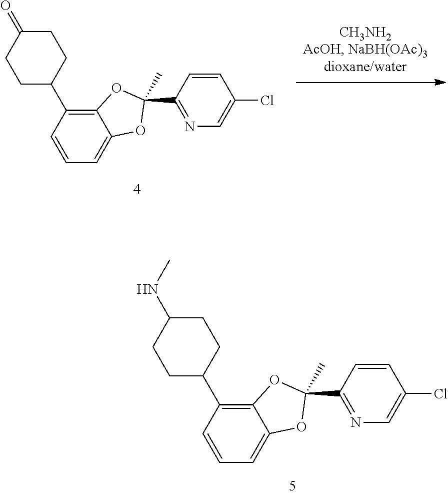 [SMILES: C[C@]1(c2ccc(Cl)cn2)Oc2cccc(C3CCC(=O)CC3)c2O1 ; CNC1CCC(c2cccc3c2O[C@@](C)(c2ccc(Cl)cn2)O3)CC1]

Equip a 250 mL three-necked round bottom flask, thermometer and N₂ balloon. DCE (20 mL) was charged to the three-necked round bottom flask, then(S)-4-(2-(5-chloropyridin-2-yl)-2-methylbenzo[d][1,3]dioxol-4-yl)cyclohexan-1-one 4 (2.0 g, 5.82 mmol, 1 eq) was added to the mixture at 20° C. CH₃NH₂ (2 M, 29 mL, 10 eq) and AcOH (524 mg, 8.73 mmol, 500 μL, 1.5 eq) was added dropwise to the reaction mixture at 20° C. After the addition, the mixture was stirred at 20° C. for 3 h. NaBH(OAc)₃ (3.70 g, 17.5 mmol, 3 eq) was added to the reaction mixture at 20° C. After the addition, the mixture was stirred at 20° C. for 4 h. LCMS showed the starting material was consumed completely. After 7 h, the reaction mixture was added to sat. Na₂CO₃ to adjust pH=8-9 at 20° C. The mixture was extracted with ethyl acetate (30 mL*3). Then organic phase was combined and washed by brine (20 mL*2), dried over anhydrous Na₂SO₄ and filtered. The organic phase was concentrated under reduced pressure at 35° C., then dried in vacuum to provide product (2 g, 5.57 mmol, 96% yield) as a brown oil.

LCMS: RT=0.369 min, MS cal.: 358.1, [M+H]⁺=359.3

### Preparation of Intermediate (1R,4s)-4-((S)-2-(5-chloropyridin-2-yl)-2-methylbenzo[d][1,3]dioxol-4-yl)-N-methylcyclohexan-1-amine (6) and Intermediate (1S,4r)-4-((S)-2-(5-chloropyridin-2-yl)-2-methylbenzo[d][1,3]dioxol-4-yl)-N-methylcyclohexan-1-amine (7)

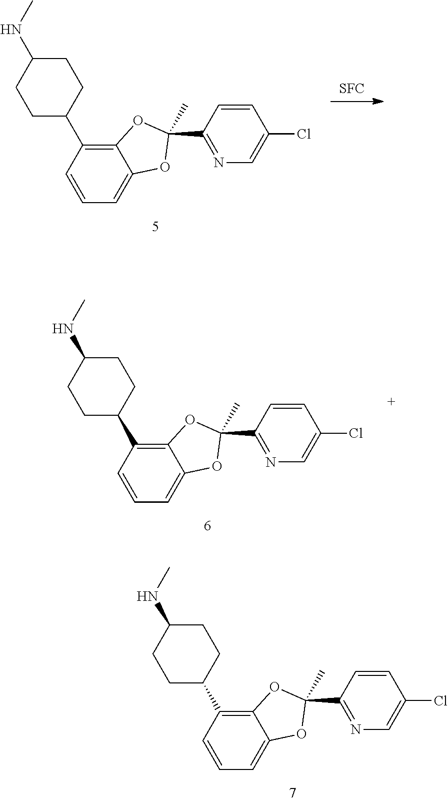 [SMILES: CNC1CCC(c2cccc3c2O[C@@](C)(c2ccc(Cl)cn2)O3)CC1 ; "SFC" ; CN[C@H]1CC[C@@H](c2cccc3c2O[C@@](C)(c2ccc(Cl)cn2)O3)CC1 ; CN[C@H]1CC[C@H](c2cccc3c2O[C@@](C)(c2ccc(Cl)cn2)O3)CC1]

(S)-4-(2-(5-Chloropyridin-2-yl)-2-methylbenzo[d][1,3]dioxol-4-yl)-N-methylcyclohexan-1-amine 5 was separated by SFC (column: DAICEL CHIRALCEL OD 250 mm*50 mm, 0 um); mobile phase: [CO₂-EtOH 0.1% NH₃H₂O)]; B %: 25%, isocratic elution mode) for 1^{st} round and by SFC (column: DAICEL CHIRALCEL OD (250 mm*30 mm, 10 um); mobile phase: [CO₂—EtOH (0.1% NH₃H₂O)]; B %: 20%, isocratic elution mode) for 2^{nd} round. (1R,4s)-4-((S)-2-(5-Chloropyridin-2-yl)-2-methylbenzo[d][1,3]dioxol-4-yl)-N-methylcyclohexan-1-amine (6) (1.2 g, 3.34 mmol, 67% yield) was obtained as a brown solid. (1S,4r)-4-((S)-2-(5-Chloropyridin-2-yl)-2-methylbenzo[d][1,3]dioxol-4-yl)-N-methylcyclohexan-1-amine (7) (180 mg. 32 μmol, 50% yield) was obtained as a brown solid.

Compound 6 SFC: ee %=100.00%

Compound 7 SFC: ee %=99.58%

¹H NMR (400 MHz, METHANOL-d₄) δ=8.59 (d, J=2.0 Hz, 1H), 7.86 (dd, J=2.4, 8.4 Hz, 1H), 7.65 (d, J=8.4 Hz, 1H), 6.84-6.74 (m, 2H), 6.68-6.66 (m, 1H), 2.80-2.76 (m, 1H), 2.48 (s, 3H), 2.47-2.42 (m, 1H), 2.01 (s, 3H), 1.93-1.75 (m, 4H), 1.70-1.53 (m, 4H)

¹H NMR (400 MHz, CHLOROFORM-d) δ=8.65 (d, J=2.4 Hz, 1H), 7.69 (dd, J=2.6, 8.4 Hz, 1H), 7.58 (dd, J=0.6, 8.4 Hz, 1H), 6.82-6.74 (m, 1H), 6.73-6.66 (m, 2H), 2.77-2.64 (m, 1H), 2.49 (s, 3H), 2.47-2.42 (m, 1H), 2.15-2.10 (m, 1H), 2.06 (s, 3H), 2.02-1.84 (m, 3H), 1.69-1.53 (m, 2H), 1.33-1.18 (m, 2H).

### Preparation of Intermediate Methyl (S)-3-methoxy-4-nitro-5-((oxetan-2-ylmethyl)amino)benzoate (9)

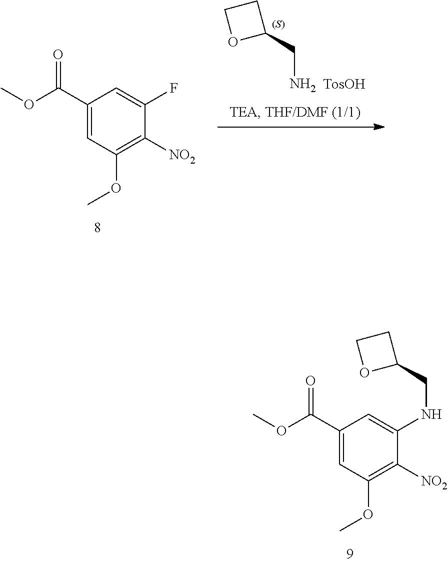

A mixture of methyl 3-fluoro-5-methoxy-4-nitrobenzoate 8 (1 g, 4.36 mmol, 1 eq), (S)-oxetan-2-ylmethanamine (1.36 g, 5.24 mmol, 1.2 eq, p-TSA) and TEA (1.32 g, 13.1 mmol, 1.82 mL, 3 eq) in THF (5 mL) and DMF (5 mL) was degassed and purged with N₂ for 3 times, and then the mixture was stirred at 60° C. for 12 h under N₂ atmosphere. The reaction mixture was quenched by H₂O 10 mL at 20° C., extracted with DCM 30 mL (10 mL*3). The combined organic layers were washed with brine 20 mL (10 mL*2), dried over Na₂SO₄, filtered, and concentrated under reduced pressure to give a residue. The residue was purified by column chromatography (SiO₂, Petroleum ether/Ethyl acetate=100/1 to 0/1) to provide product (630 mg, 2.13 mmol, 49% yield) as a yellow solid.

¹H NMR (400 MHz, CHLOROFORM-d) δ=7.14 (d, J=1.2 Hz, 1H), 6.97 (d, J=1.2 Hz, 1H), 6.25 (br s, 1H), 5.14-5.04 (m, 1H), 4.74-4.70 (m, 1H), 4.59-4.56 (m, 1H), 3.95 (s, 3H), 3.94 (s, 3H), 3.49 (d, J=4.4 Hz, 2H), 2.79-2.66 (m, 1H), 2.59-2.56 (m, 1H)

### Preparation of Intermediate Methyl (S)-4-amino-3-methoxy-5-((oxetan-2-ylmethyl)amino)benzoate (10)

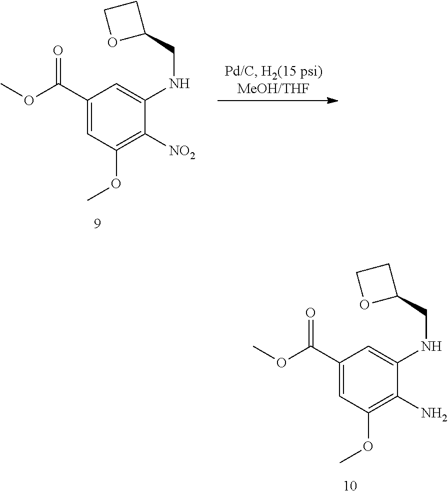 [SMILES: COC(=O)c1cc(NC[C@@H]2CCO2)c([N+](=O)[O-])c(OC)c1 ; "Pd/C, H2(15 psi)MeOH/THF" ; COC(=O)c1cc(NC[C@@H]2CCO2)c(N)c(OC)c1]

Equip a 50 mL round-bottom flask, MeOH (3 mL) and THF (3 mL) was charged to the flask, then methyl (S)-3-methoxy-4-nitro-5-((oxetan-2-ylmethyl)amino)benzoate 9 (630 mg, 2.13 mmol, 1 eq) was added to the mixture at 20° C. for 1 min. At 20° C., Pd/C (200 mg, 10%) was added to the reaction mixture within 1 min. After the addition, the suspension was degassed and purged with H₂ for 3 times. Then the mixture was stirred at 20° C. for 1 h under H₂ (15 psi). LCMS showed the starting material was consumed completely. The suspension was filtered through kieselguhr and the filter cake was washed with MeOH/THF=1/1 (8 mL*3). The combined filtrates were concentrated under reduced pressure to give the residue. The residue was purified by column chromatography (SiO₂, Petroleum ether/Ethyl acetate=100/1 to 0/1) to provide product (550 mg, 2.07 mmol, 97% yield) as a white solid.

LCMS: RT=0.345 min, MS cal.: 266.1, [M+H]⁺=267.3

### Preparation of Intermediate Methyl (S)-2-amino-4-methoxy-1-(oxetan-2-ylmethyl)-1H-benzo[d]imidazole-6-carboxylate (11)

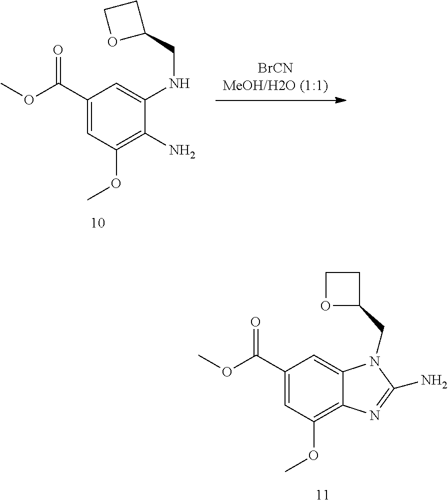 [SMILES: COC(=O)c1cc(NC[C@@H]2CCO2)c(N)c(OC)c1 ; "BrCNMeOH/H2O (1:1)" ; COC(=O)c1cc(OC)c2nc(N)n(C[C@@H]3CCO3)c2c1]

Equip a 100 mL three-necked round bottom flask, thermometer and N₂ balloon. MeOH (5 mL) and H₂O (5 mL) was charged to the three-necked round bottom flask, then methyl (S)-4-amino-3-methoxy-5-((oxetan-2-ylmethyl)amino)benzoate 10 (550 mg, 2.07 mmol, 1 eq) was added to the mixture at 20° C. BrCN (328 mg, 3.10 mmol, 227 μL, 1.5 eq) was added to the reaction mixture at 20° C. After the addition, the mixture was stirred at 50° C. for 2 h. The reaction mixture was adjusted to pH=8 with sat. Na₂CO₃ at 20° C., and then diluted with H₂O (10 mL) and extracted with ethyl acetate (10 mL*3). The combined organic layers were washed with brine (10 mL), dried over Na₂SO₄, filtered, and concentrated under reduced pressure to give a residue. The residue was purified by column chromatography (SiO₂, Petroleum ether/Ethyl acetate=50/1 to 0/1) to provide product (380 mg, 1.30 mmol, 63% yield) as a white solid.

LCMS: RT=0.302 min, MS cal.: 291.1, [M+H]⁺=292.2

¹H NMR (400 MHz, CHLOROFORM-d) δ=7.46 (d, J=1.0 Hz, 1H), 7.40 (d, J=1.0 Hz, 1H), 5.46 (br s, 2H), 5.33-5.25 (m, 1H), 4.70-4.61 (m, 1H), 4.39-4.36 (m, 1H), 4.18 (t, J=2.8 Hz, 2H), 4.04 (s, 3H), 3.93 (s, 3H), 2.80-2.66 (m, 1H), 2.46-2.41 (m, 1H)

### Preparation of Intermediate Methyl (S)-2-chloro-4-methoxy-1-(oxetan-2-ylmethyl)-1H-benzo[d]imidazole-6-carboxylate (12)

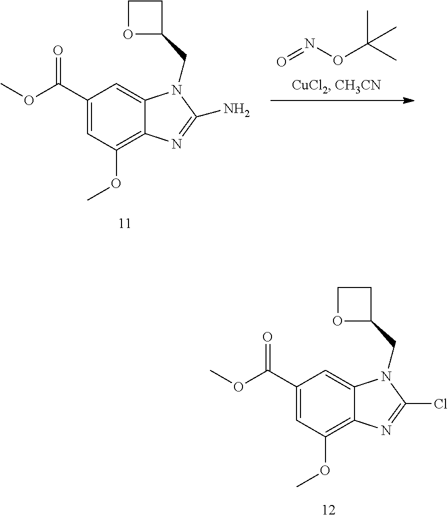

A mixture of methyl (S)-2-amino-4-methoxy-1-(oxetan-2-ylmethyl)-1H-benzo[d]imidazole-6-carboxylate 11 (1.2 g, 4.12 mmol, 1 eq), CuCl₂ (554 mg, 4.12 mmol, 1 eq) and tert-butyl nitrite (637 mg, 6.18 mmol, 735 μL, 1.5 eq) in CH₃CN (10 mL) was degassed and purged with N₂ for 3 times, and then the mixture was stirred at 65° C. for 2 h under N₂ atmosphere. The reaction mixture was quenched by addition H₂O 5 mL at 20° C., extracted with ethyl acetate 15 mL (5 mL*3). The combined organic layers were washed with brine 10 mL (5 mL*2), dried over Na₂SO₄, filtered, and concentrated under reduced pressure to give a residue. The residue was purified by column chromatography (SiO₂, Petroleum ether/Ethyl acetate=50/1 to 0/1) to provide product (650 mg, 2.09 mmol, 50.78% yield) as a white solid.

LCMS: RT=0.390 min, MS cal.: 310.1, [M+H]⁺=311.0

¹H NMR (400 MHz, CHLOROFORM-d) δ=7.84 (d, J=1.2 Hz, 1H), 7.42 (d, J=1.2 Hz, 1H), 5.25-5.14 (m, 1H), 4.62-4.60 (m, 1H), 4.47-4.44 (m, 2H), 4.39-4.36 (m, 1H), 4.07 (s, 3H), 3.95 (s, 3H), 2.83-2.69 (m, 1H), 2.57-2.42 (m, 1H)

### Preparation of Intermediate Methyl 2-(((1R,4R)-4-((S)-2-(5-chloropyridin-2-yl)-2-methylbenzo[d][1,3]dioxol-4-yl)cyclohexyl)(methyl)amino)-4-methoxy-1-(((S)-oxetan-2-yl)methyl)-1H-benzo[d]imidazole-6-carboxylate (13)

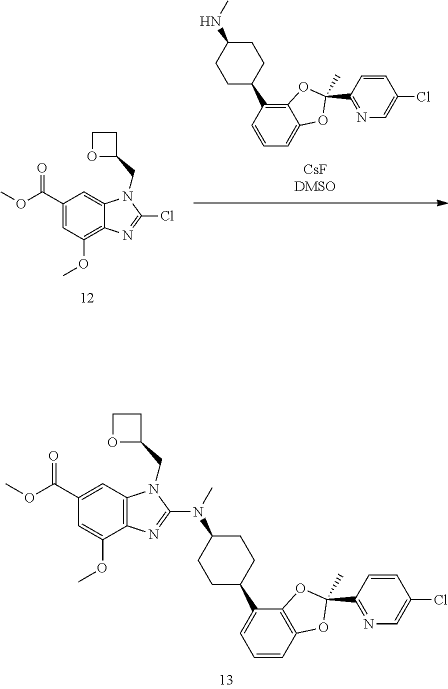 [SMILES: COC(=O)c1cc(OC)c2nc(Cl)n(C[C@@H]3CCO3)c2c1 ; CN[C@H]1CC[C@@H](c2cccc3c2O[C@@](C)(c2ccc(Cl)cn2)O3)CC1 ; COC(=O)c1cc(OC)c2nc(N(C)[C@H]3CC[C@@H](c4cccc5c4O[C@@](C)(c4ccc(Cl)cn4)O5)CC3)n(C[C@@H]3CCO3)c2c1]

A mixture of methyl (S)-2-chloro-4-methoxy-1-(oxetan-2-ylmethyl)-1H-benzo[d]imidazole-6-carboxylate 12 (50 mg, 161 μmol, 1 eq), 6 (57.7 mg, 161 μmol, 1 eq) and CsF (73.3 mg, 483 μmol, 17.8 μL, 3 eq) in DMSO (2 mL) was degassed and purged with N₂ for 3 times, and then the mixture was stirred at 100° C. for 12 h under N₂ in glove box. LCMS showed the starting material was consumed completely. The reaction mixture was quenched by addition H₂O 2 mL at 20° C., extracted with ethyl acetate 15 mL (3 mL*5). The combined organic layers were washed with brine 10 mL (5 mL*2), dried over Na₂SO₄, filtered, and concentrated under reduced pressure to give a residue. The residue was purified by column chromatography (SiO₂, Petroleum ether/Ethyl acetate=9/1 to 1/1) to provide product (30 mg, 47.4 μmol, 29% yield) as a white solid.

LCMS: RT=2.474 min, MS cal.: 632.2, [M+H]⁺=633.3

¹H NMR (400 MHz, METHANOL-d4) δ=8.62 (d, J=2.0 Hz, 1H), 7.91 (d, J=1.6 Hz, 1H), 7.88 (dd, J=2.4, 8.4 Hz, 1H), 7.65 (d, J=8.4 Hz, 1H), 7.41 (d, J=1.0 Hz, 1H), 6.84-6.79 (m, 2H), 6.73-6.68 (m, 1H), 5.32-5.24 (m, 1H), 4.69-4.57 (m, 2H), 4.53-4.50 (m, 1H), 4.39-4.36 (m, 1H), 4.01 (s, 3H), 3.92 (s, 3H), 3.74-3.71 (m, 1H), 2.91-2.81 (m, 4H), 2.80-2.72 (m, 1H), 2.56-2.46 (m, 1H), 2.15-2.05 (m, 2H), 2.04 (s, 3H), 2.01-1.97 (m, 2H), 1.75-1.55 (m, 4H)

### Preparation of 2-(((1R,4R)-4-((S)-2-(5-chloropyridin-2-yl)-2-methylbenzo[d][1,3]dioxol-4-yl)cyclohexyl)(methyl)amino)-4-methoxy-1-(((S)-oxetan-2-yl)methyl)-1H-benzo[d]imidazole-6-carboxylic acid, Compound 79 (14)

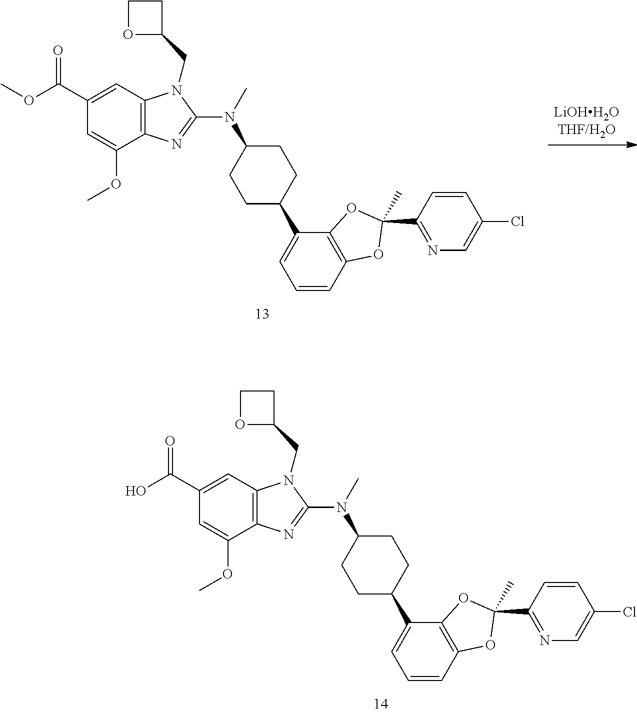 [SMILES: COC(=O)c1cc(OC)c2nc(N(C)[C@H]3CC[C@@H](c4cccc5c4O[C@@](C)(c4ccc(Cl)cn4)O5)CC3)n(C[C@@H]3CCO3)c2c1 ; COc1cc(C(=O)O)cc2c1nc(N(C)[C@H]1CC[C@@H](c3cccc4c3O[C@@](C)(c3ccc(Cl)cn3)O4)CC1)n2C[C@@H]1CCO1]

A mixture of methyl 2-(((1R,4R)-4-((S)-2-(5-chloropyridin-2-yl)-2-methylbenzo[d][1,3]dioxol-4-yl)cyclohexyl)(methyl)amino)-4-methoxy-1-(((S)-oxetan-2-yl)methyl)-1H-benzo[d]imidazole-6-carboxylate 13 (40.0 mg, 63.2 μmol, 1 eq) and LiOH·H₂O (3.98 mg, 94.8 μmol, 1.5 eq) in THF (0.7 mL) and H₂O (0.3 mL) was degassed and purged with N₂ for 3 times, and then the mixture was stirred at 25° C. for 12 h under N₂ atmosphere. LCMS showed the starting material was consumed completely. The reaction was purified by prep-HPLC (column: Waters Xbridge Prep OBD C18 150*40 mm*10 um; mobile phase: [H₂O (10 mM NH₄HCO₃)—CH₃CN]; gradient: 20%-70% B over 8.0 min) to provide product (25.1 mg, 40.5 μmol, 64% yield) as a white solid.

LCMS: RT=2.686 min, MS cal.: 619.1, [M+H]⁺=619.3

HPLC: RT=12.254 min, purity: 99.27%

¹H NMR (400 MHz, METHANOL-d4) δ=8.62 (d, J=2.0 Hz, 1H), 7.93-7.84 (m, 2H), 7.65 (d, J=8.6 Hz, 1H), 7.43 (d, J=1.0 Hz, 1H), 6.85-6.78 (m, 2H), 6.73-6.71 (m, 1H), 5.34-5.25 (m, 1H), 4.71-4.59 (m, 2H), 4.55-4.47 (m, 1H), 4.39-4.37 (m, 1H), 4.01 (s, 3H), 3.73-3.71 (m, 1H), 2.89-2.86 (m, 1H), 2.84 (s, 3H), 2.81-2.73 (m, 1H), 2.55-2.52 (m, 1H), 2.15-2.05 (m, 2H), 2.04 (s, 3H), 2.02-1.91 (m, 2H), 1.77-1.54 (m, 4H)

### Example A20. —Preparation of Compound 80. 2-(((1S,4S)-4-((S)-2-(5-Chloropyridin-2-yl)-2-methylbenzo[d][1,3]dioxol-4-yl)cyclohexyl)(methyl)amino)-4-methoxy-1-(((S)-oxetan-2-yl)methyl)-1H-benzo[d]imidazole-6-carboxylic acid (Compound 80) (3)

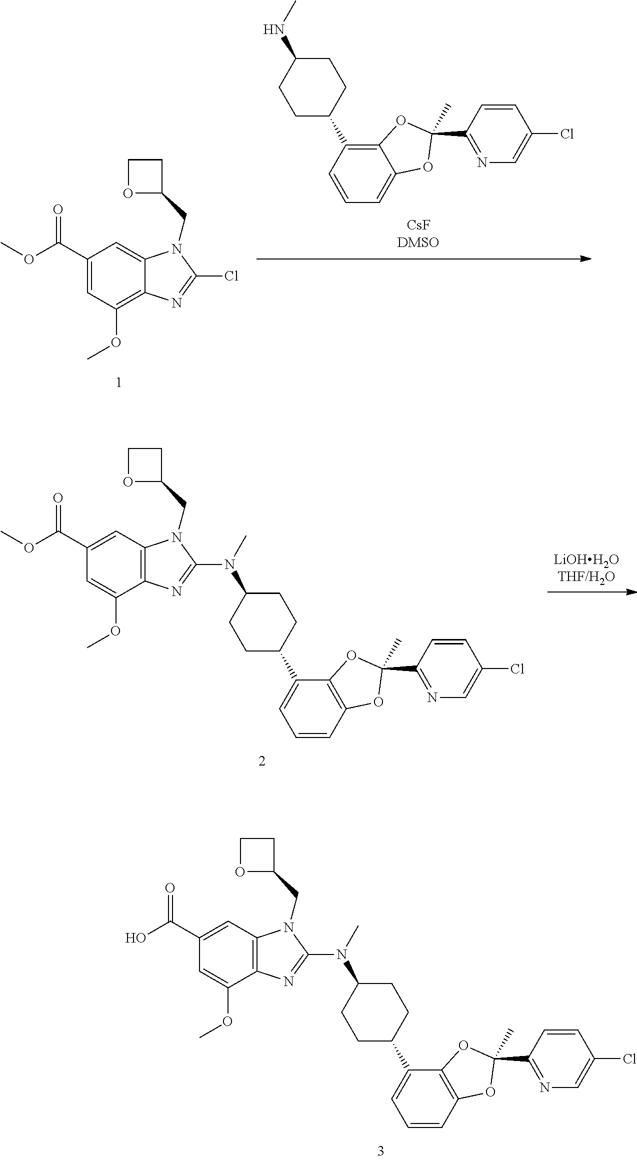 [SMILES: COC(=O)c1cc(OC)c2nc(Cl)n(C[C@@H]3CCO3)c2c1 ; CN[C@H]1CC[C@H](c2cccc3c2O[C@@](C)(c2ccc(Cl)cn2)O3)CC1 ; COC(=O)c1cc(OC)c2nc(N(C)[C@H]3CC[C@H](c4cccc5c4O[C@@](C)(c4ccc(Cl)cn4)O5)CC3)n(C[C@@H]3CCO3)c2c1 ; COc1cc(C(=O)O)cc2c1nc(N(C)[C@H]1CC[C@H](c3cccc4c3O[C@@](C)(c3ccc(Cl)cn3)O4)CC1)n2C[C@@H]1CCO1]

### Preparation of Intermediate Methyl 2-(((1S,4S)-4-((S)-2-(5-chloropyridin-2-yl)-2-methylbenzo[d][1,3]dioxol-4-yl)cyclohexyl)(methyl)amino)-4-methoxy-1-(((S)-oxetan-2-yl)methyl)-1H-benzo[d]imidazole-6-carboxylate (2)

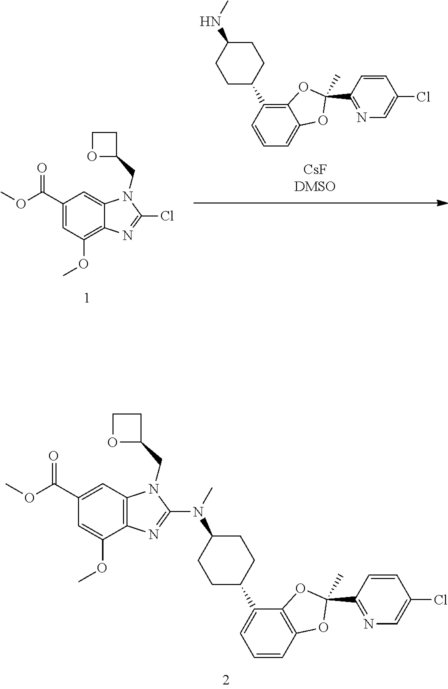 [SMILES: COC(=O)c1cc(OC)c2nc(Cl)n(C[C@@H]3CCO3)c2c1 ; CN[C@H]1CC[C@H](c2cccc3c2O[C@@](C)(c2ccc(Cl)cn2)O3)CC1 ; COC(=O)c1cc(OC)c2nc(N(C)[C@H]3CC[C@H](c4cccc5c4O[C@@](C)(c4ccc(Cl)cn4)O5)CC3)n(C[C@@H]3CCO3)c2c1]

To a solution of methyl (S)-2-chloro-4-methoxy-1-(oxetan-2-ylmethyl)-1H-benzo[d]imidazole-6-carboxylate 1 (43.3 mg, 139 μmol, 1 eq) and (15,4r)-4-((S)-2-(5-chloropyridin-2-yl)-2-methylbenzo[d][1,3]dioxol-4-yl)-N-methylcyclohexan-1-amine (50.0 mg, 139 μmol, 1 eq) in DMSO (1 mL) was added CsF (63.5 mg, 418 μmol, 15.4 μL, 3 eq) and 4A molecular sieve (50 mg). The mixture was stirred at 100° C. for 12 h. LC-MS showed starting material was consumed completely and desired mass was detected. The reaction mixture was quenched by addition H₂O 5 mL at 20° C., extracted with Ethyl acetate 15 mL (5 mL*3). Then organic phase was combined and washed by H₂O (5 mL), dried with Na₂SO₄, filtered. The organic phase was concentrated under reduced pressure at 40° C. to give a residue. The residue was purified by column chromatography (SiO₂, Petroleum ether/Ethyl acetate=50/1 to 0/1) to provide product (50 mg, 79 μmol, 28% yield) as a white solid.

LCMS: RT=0.941 min, MS cal.: 632.2, [M+H]⁺=633.3

### Preparation of Methyl 2-(((1S,4S)-4-((S)-2-(5-chloropyridin-2-yl)-2-methylbenzo[d][1,3]dioxol-4-yl)cyclohexyl)(methyl)amino)-4-methoxy-1-(((S)-oxetan-2-yl)methyl)-1H-benzo[d]imidazole-6-carboxylate, Compound 80 (3)

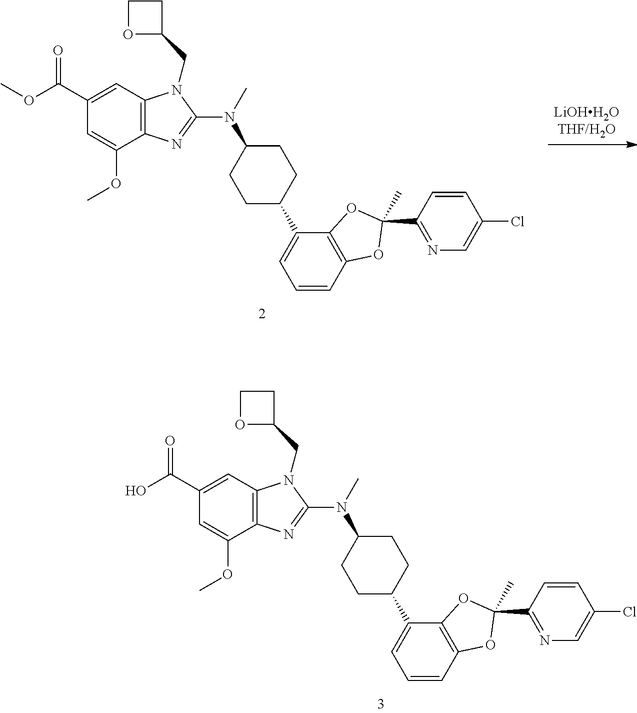 [SMILES: COC(=O)c1cc(OC)c2nc(N(C)[C@H]3CC[C@H](c4cccc5c4O[C@@](C)(c4ccc(Cl)cn4)O5)CC3)n(C[C@@H]3CCO3)c2c1 ; COc1cc(C(=O)O)cc2c1nc(N(C)[C@H]1CC[C@H](c3cccc4c3O[C@@](C)(c3ccc(Cl)cn3)O4)CC1)n2C[C@@H]1CCO1]

To a solution of methyl 2-(((1S,4S)-4-((S)-2-(5-chloropyridin-2-yl)-2-methylbenzo[d][1,3]dioxol-4-yl)cyclohexyl)(methyl)amino)-4-methoxy-1-(((S)-oxetan-2-yl)methyl)-1H-benzo[d]imidazole-6-carboxylate 2 (45 mg, 71.1 μmol, 1 eq) in THF (0.7 mL) was added LiOH·H₂O (5.97 mg, 142 μmol, 2 eq) in H₂O (0.3 mL). The mixture was stirred at 20° C. for 12 h. LC-MS showed starting material was consumed completely and desired mass was detected. The reaction was purified by reversed-phase HPLC (column: Waters Xbridge Prep OBD C18 150*40 mm*10 um; mobile phase: [H₂O (10 mM NH₄HCO₃)—CH₃CN]; gradient: 20%-70% B over 8.0 min) to provide product (26.0 mg, 42.0 μmol, 59% yield) as a white solid.

LCMS: RT=2.772 min, MS cal.: 619.1, [M+H]⁺=619.3

HPLC: RT=12.350 min, purity: 100%

¹H NMR (400 MHz, METHANOL-d₄) δ=8.60 (d, J=2.0 Hz, 1H), 7.88 (dd, J=2.4, 8.4 Hz, 1H), 7.80 (d, J=1.2 Hz, 1H), 7.65 (d, J=8.8 Hz, 1H), 7.43 (d, J=1.2 Hz, 1H), 6.81-6.65 (m, 3H), 5.36-5.33 (m, 1H), 4.66-4.63 (m, 1H), 4.57-4.48 (m, 2H), 4.31-4.28 (m, 1H), 4.00 (s, 3H), 3.71-3.61 (m, 1H), 2.94 (s, 3H), 2.86-2.75 (m, 1H), 2.74-2.64 (m, 1H), 2.62-2.52 (m, 1H), 2.10-2.03 (m, 1H), 2.01 (s, 3H), 1.95-1.92 (m, 3H), 1.85-1.65 (m, 4H)

### Example A21. —Preparation of Compound 81. 2-((4-((S)-2-(5-Chloropyridin-2-yl)-2-methylbenzo[d][1,3]dioxol-4-yl)piperidin-1-yl)methyl)-4-(2-fluoroethoxy)-1-(((S)-oxetan-2-yl)methyl)-1H-benzo[d]imidazole-6-carboxylic acid (Compound 81) (8)

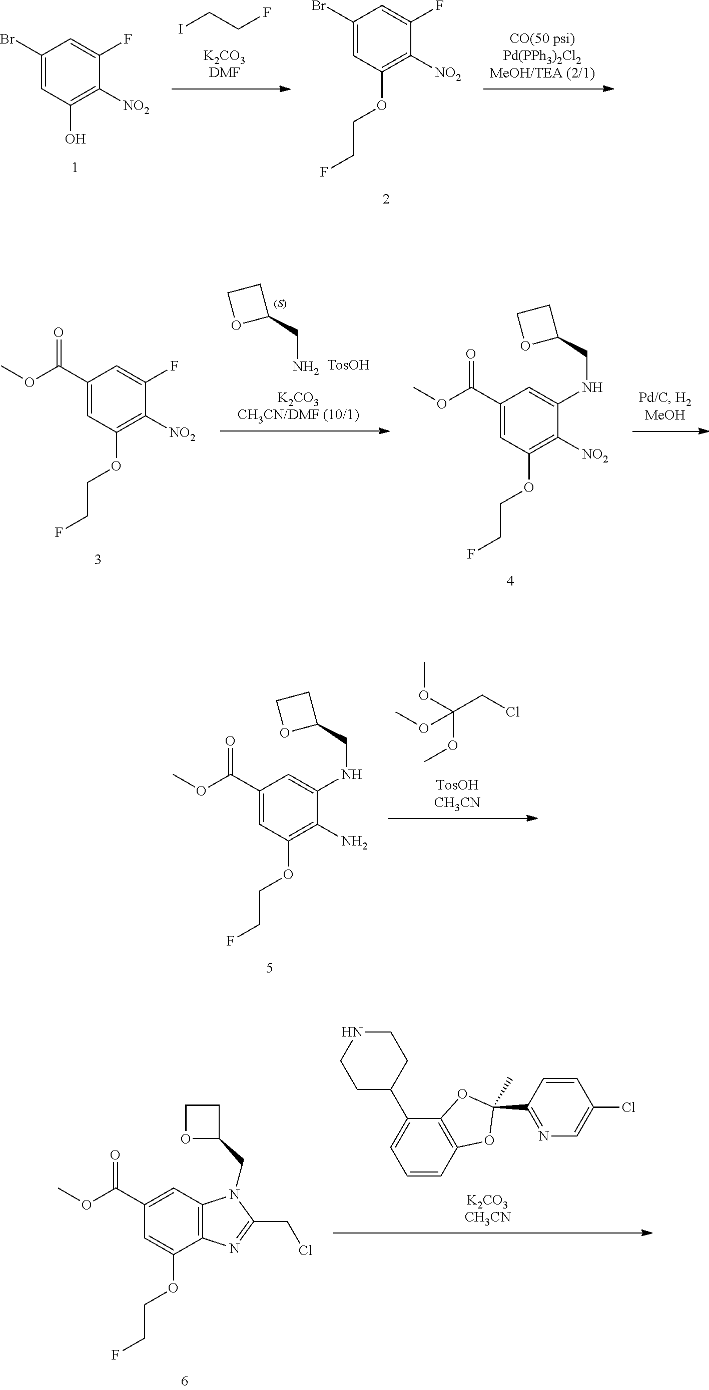

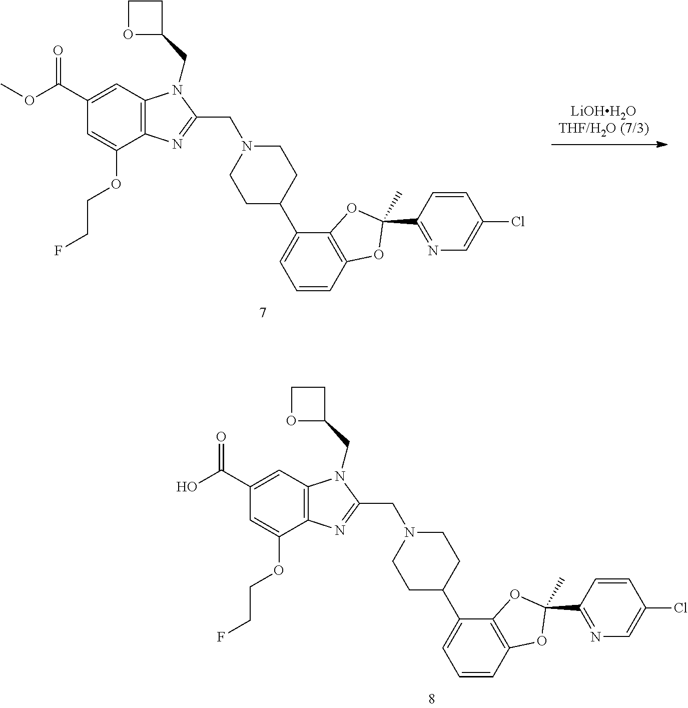 [SMILES: COC(=O)c1cc(OCCF)c2nc(CN3CCC(c4cccc5c4O[C@@](C)(c4ccc(Cl)cn4)O5)CC3)n(C[C@@H]3CCO3)c2c1 ; C[C@]1(c2ccc(Cl)cn2)Oc2cccc(C3CCN(Cc4nc5c(OCCF)cc(C(=O)O)cc5n4C[C@@H]4CCO4)CC3)c2O1]

### Preparation of 5Intermediate-Bromo-1-fluoro-3-(2-fluoroethoxy)-2-nitrobenzene (2)

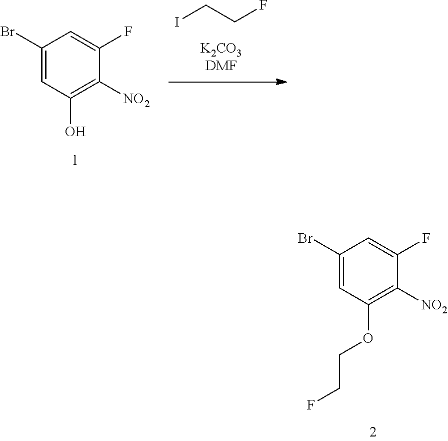

To a solution of 5-bromo-3-fluoro-2-nitrophenol, 1 (3 g, 12.7 mmol, 1 eq) in DMF (30 mL) was added K₂CO₃ (5.27 g, 38.14 mmol, 3 eq) and 1-fluoro-2-iodoethane (4.2 g, 24.2 mmol, 1.9 eq). The mixture was stirred at 50° C. for 3.5 h. TLC (Petroleum ether/Ethyl acetate=3/1, R_{f}=0.56) indicated starting material was consumed completely and one new spot formed. The reaction was clean according to TLC. The residue was poured into H₂O (60 mL). The aqueous phase was extracted with ethyl acetate (25 mL*3). Then organic phase was combined and washed by H₂O (50 mL), dried with Na₂SO₄, filtered. The organic phase was concentrated under reduced pressure at 40° C. to provide product (3.1 g, 11 mmol, 86% yield) as a white solid.

¹H NMR (400 MHz, DMSO-d6) δ=7.61 (dd, J=1.6, 9.2 Hz, 1H), 7.57 (s, 1H), 4.80-4.63 (m, 2H), 4.59-4.45 (m, 2H)

### Preparation of Intermediate Methyl 3-fluoro-5-(2-fluoroethoxy)-4-nitrobenzoate (3)

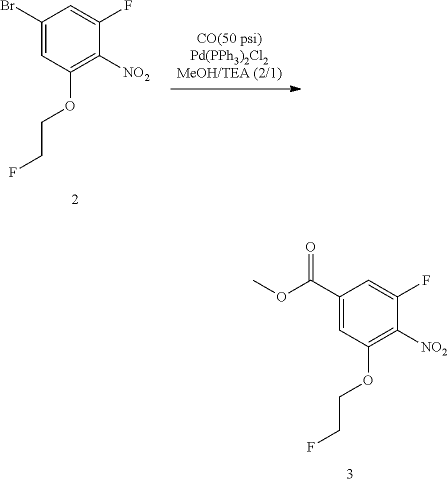 [SMILES: O=[N+]([O-])c1c(F)cc(Br)cc1OCCF ; COC(=O)c1cc(F)c([N+](=O)[O-])c(OCCF)c1]

MeOH (25 mL) and TEA (12.5 mL) was charged to the 125 mL Hydrogenated bottle, then 5-bromo-1-fluoro-3-(2-fluoroethoxy)-2-nitrobenzene 2 (2.6 g, 9.22 mmol, 1 eq) and Pd(PPh₃)₂Cl₂ (647 mg, 922 μmol, 0.1 eq) was added at 20° C. After the addition, the mixture was degassed and purged with CO for 3 times. The mixture was stirred at 80° C. under CO (50 psi) for 12 h. The residue was poured into H₂O (30 mL). The aqueous phase was extracted with DCM (20 mL*3). The combined organic phase was washed with brine (30 mL*2), dried with anhydrous Na₂SO₄, filtered, and concentrated in vacuum. The residue was purified by column chromatography (SiO₂, Petroleum ether/Ethyl acetate=1/0 to 0/1) to provide product (2.03 g, 7.77 mmol, 84% yield) as a yellow solid.

¹H NMR (400 MHz, CHLOROFORM-d) δ=7.56-7.53 (m, 2H), 4.84-7.81 (m, 2H), 4.47-4.40 (m, 2H), 3.98 (s, 3H)

### Preparation of Intermediate Methyl (S)-3-(2-fluoroethoxy)-4-nitro-5-((oxetan-2-ylmethyl)amino)benzoate (4)

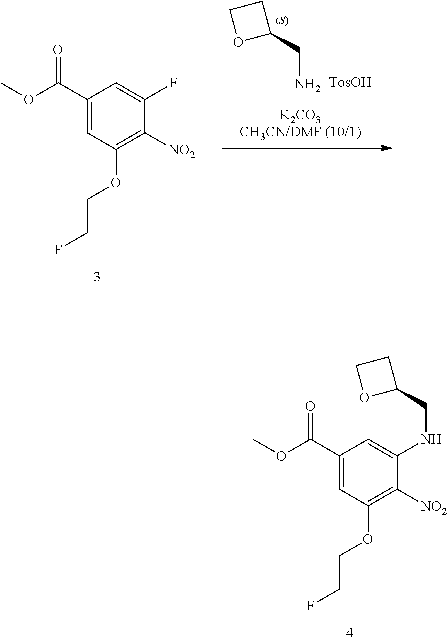

To a solution of methyl 3-fluoro-5-(2-fluoroethoxy)-4-nitrobenzoate 3 (1.06 g, 4.05 mmol, 1 eq) and(S)-oxetan-2-ylmethanamine (529 mg, 6.07 mmol, 1.5 eq) in CH₃CN (15 mL) and DMF (1.5 mL) was added K₂CO₃ (1.68 g, 12.1 mmol, 3 eq). The mixture was stirred at 60° C. for 12 h. TLC (Petroleum ether/Ethyl acetate=1/1) indicated starting material was consumed completely and one new spot formed. The reaction was clean according to TLC. The mixture was diluted with H₂O (30 mL), extracted with Ethyl acetate (50 mL*3). The combined organic layer was washed with brine 40 mL (20 mL*2), dried over Na₂SO₄, filtered and concentrated under reduced pressure to give a residue. The residue was purified by column chromatography (SiO₂, Petroleum ether/Ethyl acetate=100/0 to 85/15) to provide product (1.08 g, 3.29 mmol, 81% yield) as a yellow solid.

HPLC: RT=2.077 min, purity: 99.66%.

¹H NMR (400 MHz, CHLOROFORM-d) δ=7.17 (s, 1H), 6.96 (s, 1H), 6.18 (br t, J=5.2 Hz, 1H), 5.13-5.05 (m, 1H), 4.85-4.81 (m, 1H), 4.76-4.69 (m, 2H), 4.58-4.56 (m, 1H), 4.41-4.30 (m, 2H), 3.94 (s, 3H), 3.49 (t, J=4.8 Hz, 2H), 2.77-2.68 (m, 1H), 2.61-2.52 (m, 1H)

### Preparation of Intermediate Methyl (S)-4-amino-3-(2-fluoroethoxy)-5-((oxetan-2-ylmethyl)amino)benzoate (5)

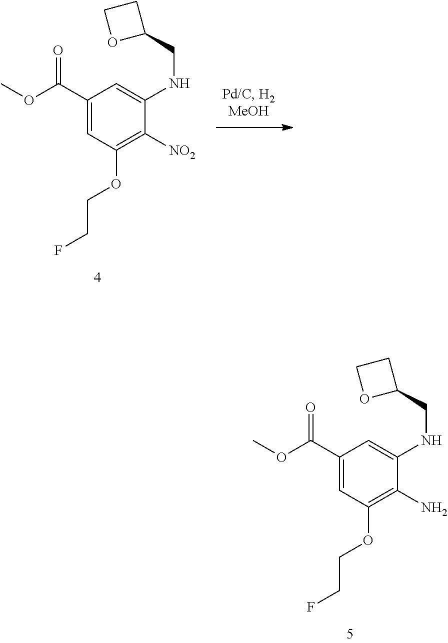 [SMILES: COC(=O)c1cc(NC[C@@H]2CCO2)c([N+](=O)[O-])c(OCCF)c1 ; COC(=O)c1cc(NC[C@@H]2CCO2)c(N)c(OCCF)c1]

THF (5 mL) was charged to the 25 mL round bottom flask under N₂ atmosphere, then Pd/C (500 mg, 10%) was added at 20° C. At 20° C., methyl (S)-3-(2-fluoroethoxy)-4-nitro-5-((oxetan-2-ylmethyl)amino)benzoate 4 (500 mg, 1.52 mmol, 1 eq) in THF (5 mL) was added to the reaction mixture at 20° C., degassed and purged with H₂ (15 psi) for 3 times. After the addition, the mixture was stirred at 20° C. under H₂ (15 psi) for 2 h. LCMS showed starting material was consumed completely and one main peak with desired m/z was detected. The reaction mixture was filtered and concentrated under reduced pressure to provide product (454 mg, 1.52 mmol, 99.9% yield) as a yellow oil.

LCMS: RT=0.348 min, MS cal.: 298.1, 299.1, [M+H]⁺=299.1

### Preparation of Intermediate Methyl (S)-2-(chloromethyl)-4-(2-fluoroethoxy)-1-(oxetan-2-ylmethyl)-1H-benzo[d]imidazole-6-carboxylate (6)

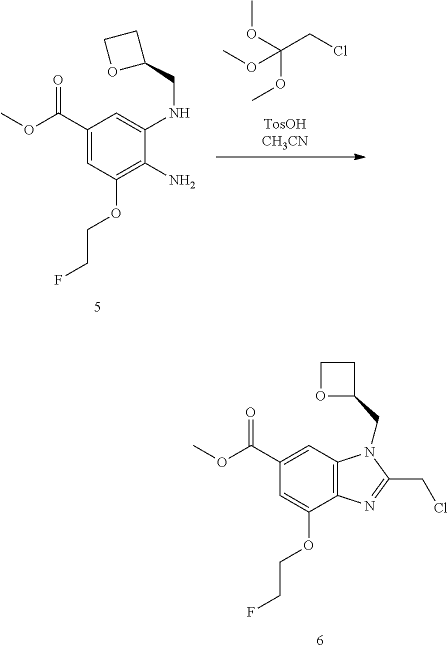

To a solution of methyl (S)-4-amino-3-(2-fluoroethoxy)-5-((oxetan-2-ylmethyl)amino)benzoate 5 (500 mg, 1.68 mmol, 1 eq) in CH₃CN (10 mL) was added TosOH (14.4 mg, 83.8 umol, 0.05 eq) and 2-chloro-1,1,1-trimethoxyethane (337 mg, 2.18 mmol, 294 uL, 1.3 eq). The mixture was stirred at 60° C. for 2 h. LCMS showed starting material was consumed completely and one main peak with desired mass was detected. The mixture was concentrated in vacuum to get a residue. The residue was purified by column chromatography (SiO₂, Petroleum ether/Ethyl acetate=3/1 to 0/1) to provide product (450 mg, 1.26 mmol, 75% yield) as a yellow oil.

LCMS: RT=0.388 min, MS cal.: 356.1, [M+H]⁺=356.9

### Preparation of Intermediate Methyl 2-((4-((S)-2-(5-chloropyridin-2-yl)-2-methylbenzo[d][1,3]dioxol-4-yl)piperidin-1-yl)methyl)-4-(2-fluoroethoxy)-1-(((S)-oxetan-2-yl)methyl)-1H-benzo[d]imidazole-6-carboxylate (7)

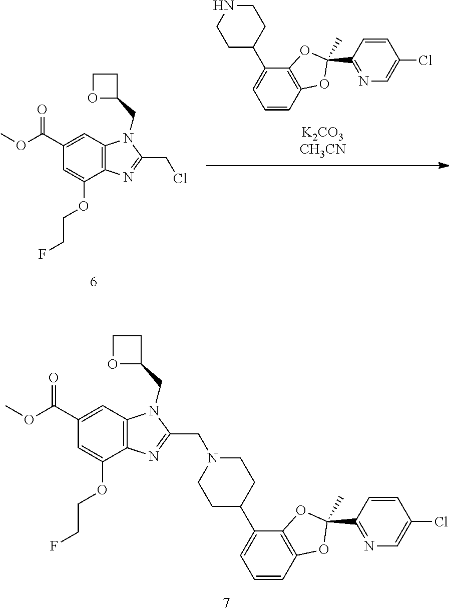 [SMILES: COC(=O)c1cc(OCCF)c2nc(CCl)n(C[C@@H]3CCO3)c2c1 ; C[C@]1(c2ccc(Cl)cn2)Oc2cccc(C3CCNCC3)c2O1 ; COC(=O)c1cc(OCCF)c2nc(CN3CCC(c4cccc5c4O[C@@](C)(c4ccc(Cl)cn4)O5)CC3)n(C[C@@H]3CCO3)c2c1]

To a solution of (S)-5-chloro-2-(2-methyl-4-(piperidin-4-yl)benzo[d][1,3]dioxol-2-yl)pyridine (211 mg, 420 umol, 1 eq, p-TSA) in CH₃CN (5 mL) was added K₂CO₃ (174.32 mg, 1.26 mmol, 3 eq) and methyl (S)-2-(chloromethyl)-4-(2-fluoroethoxy)-1-(oxetan-2-ylmethyl)-1H-benzo[d]imidazole-6-carboxylate 6 (150 mg, 420 μmol, 1 eq). The mixture was stirred at 60° C. for 2 h. LCMS showed starting material was consumed completely and one main peak with desired mass was detected. The mixture was poured into H₂O (10 mL) and extracted with ethyl acetate (5 mL*2), the organic phase was washed with brine (10 mL*2), dried over Na₂SO₄, filtered and concentrated to get a residue. The crude product was purified by column chromatography (SiO₂, Petroleum ether/Ethyl acetate=10/1 to 0/1) to provide product (160 mg, 246 μmol, 58% yield) as a yellow solid.

LCMS: RT=0.452 min, MS cal.: 650.2, [M+H]⁺=651.3

### Preparation of 2-((4-((S)-2-(5-Chloropyridin-2-yl)-2-methylbenzo[d][1,3]dioxol-4-yl)piperidin-1-yl)methyl)-4-(2-fluoroethoxy)-1-(((S)-oxetan-2-yl)methyl)-1H-benzo[d]imidazole-6-carboxylic acid Compound 81 (8)

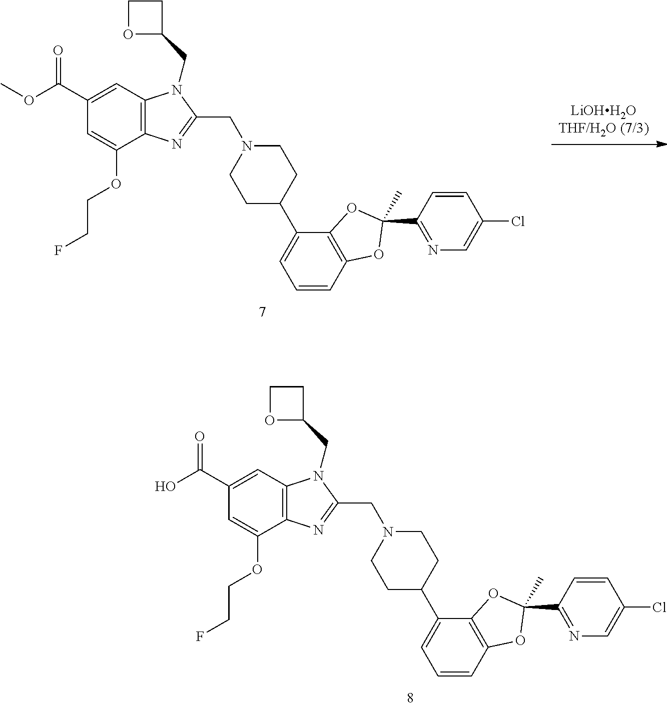 [SMILES: COC(=O)c1cc(OCCF)c2nc(CN3CCC(c4cccc5c4O[C@@](C)(c4ccc(Cl)cn4)O5)CC3)n(C[C@@H]3CCO3)c2c1 ; C[C@]1(c2ccc(Cl)cn2)Oc2cccc(C3CCN(Cc4nc5c(OCCF)cc(C(=O)O)cc5n4C[C@@H]4CCO4)CC3)c2O1]

To a solution of methyl 2-((4-((S)-2-(5-chloropyridin-2-yl)-2-methylbenzo[d][1,3]dioxol-4-yl)piperidin-1-yl)methyl)-4-(2-fluoroethoxy)-1-(((S)-oxetan-2-yl)methyl)-1H-benzo[d]imidazole-6-carboxylate 7 (160 mg, 246 μmol, 1 eq) in THF (1 mL) was added LiOH·H₂O (15.5 mg, 369 umol, 1.5 eq) in H₂O (1 mL). The mixture was stirred at 20° C. for 12 h. LC-MS showed starting material was consumed completely and one main peak with desired mass was detected. The mixture was filtered and the filtrate was purified by Prep-HPLC (column: Phenomenex luna C18 100*40 mm*3 um; mobile phase: [H₂O (0.2% FA)-ACN]; gradient: 20%-50% B over 8.0 min) to provide product (56 mg, 87.9 μmol, 36% yield) as a white solid.

LCMS: RT=2.578 min, MS cal.: 637.2, [2/M]⁺=319.1

HPLC: RT=6.749 min, purity: 99.87%

¹H NMR (400 MHz, DMSO-d6) δ=8.75-8.67 (m, 1H), 8.04-7.96 (m, 1H), 7.94-7.89 (m, 1H), 7.62-7.55 (m, 1H), 7.30-7.23 (m, 1H), 6.86-6.68 (m, 3H), 5.14-5.02 (m, 1H), 4.88-4.86 (m, 1H), 4.80-4.68 (m, 2H), 4.66-4.57 (m, 1H), 4.55-4.41 (m, 3H), 4.38-4.30 (m, 1H), 3.96-3.87 (m, 1H), 3.81-3.69 (m, 1H), 3.04-2.94 (m, 1H), 2.83-2.79 (m, 1H), 2.72-2.63 (m, 1H), 2.40-2.33 (m, 2H), 2.24-2.14 (m, 2H), 2.04 (s, 3H), 1.82-1.65 (m, 4H).

### Example A22. —Preparation of Compound 82. 2-((4-(6-((4-Cyano-2-fluorobenzyl)oxy)pyridin-2-yl)piperidin-1-yl)methyl)-4-hydroxy-1-(thiazol-5-ylmethyl)-1H-benzo[d]imidazole-6-carboxylic acid (Compound 82) (8)

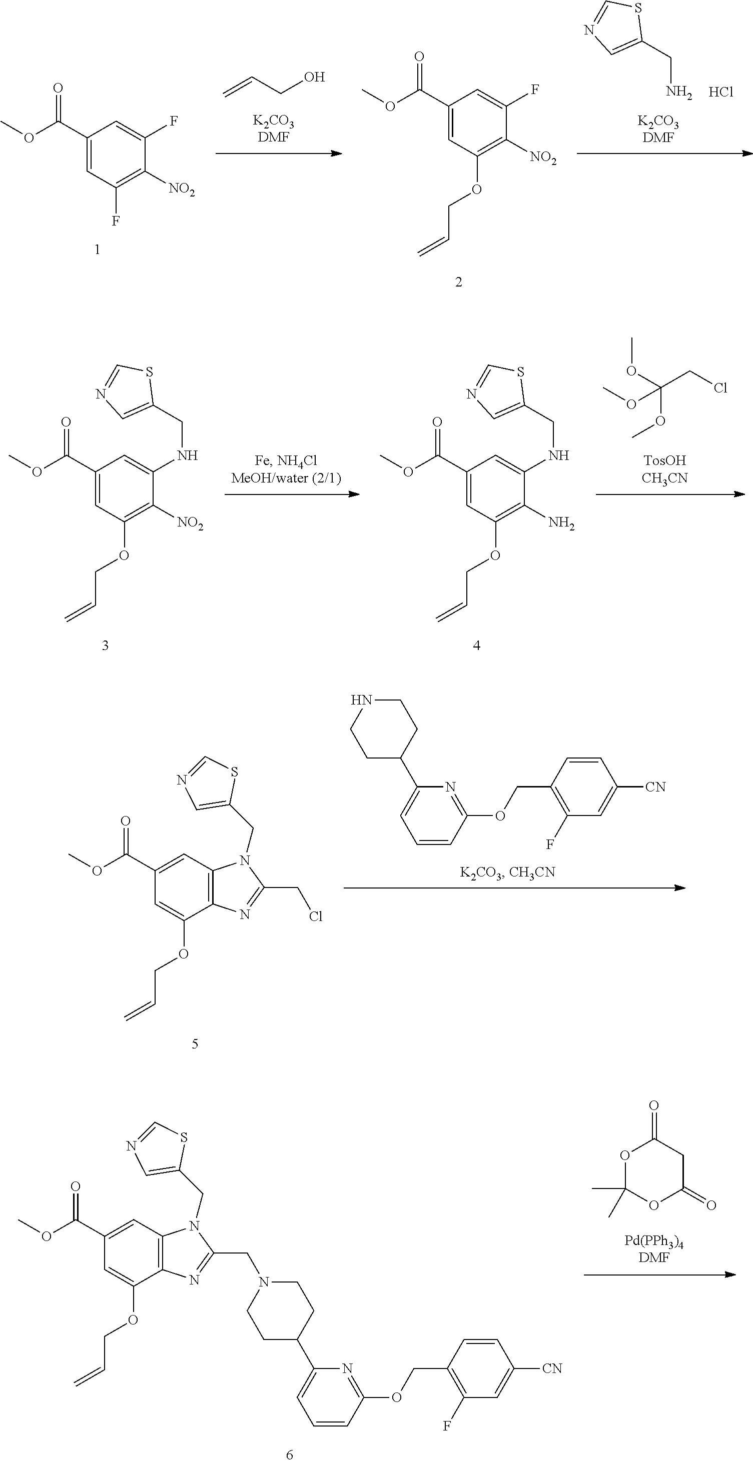

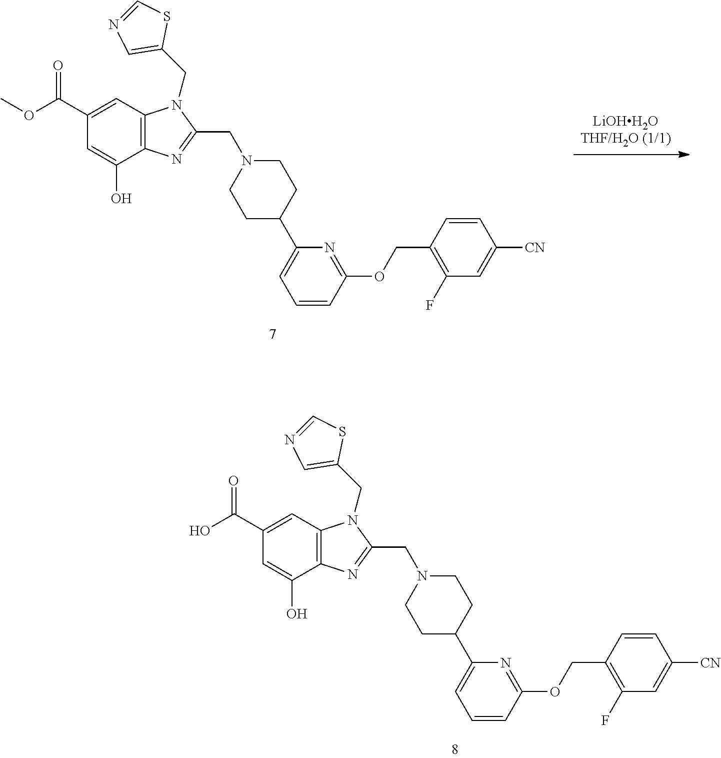 [SMILES: COC(=O)c1cc(O)c2nc(CN3CCC(c4cccc(OCc5ccc(C#N)cc5F)n4)CC3)n(Cc3cncs3)c2c1 ; N#Cc1ccc(COc2cccc(C3CCN(Cc4nc5c(O)cc(C(=O)O)cc5n4Cc4cncs4)CC3)n2)c(F)c1]

### Preparation of Intermediate Methyl 3-(allyloxy)-5-fluoro-4-nitrobenzoate (2)

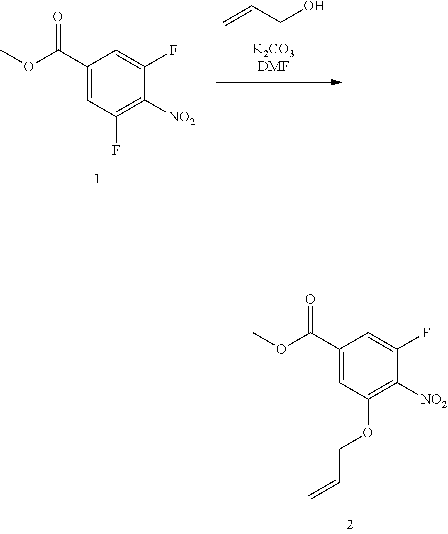

Equip a 250 mL three-necked round bottom flask and thermometer, N₂ balloon. DMF (50 mL) was charged to the 250 mL three-necked round bottom flask, then methyl 3,5-difluoro-4-nitrobenzoate 1 (5 g, 23.0 mmol, 1 eq) and K₂CO₃ (9.55 g, 69.1 mmol, 3 eq) was added to the mixture at 20° C. At 20° C., prop-2-en-1-ol (1.47 g, 25.3 mmol, 1.72 mL, 1.1 eq) was added dropwise to the reaction mixture at 20° C. within 1 min. After the addition, the mixture was stirred at 60° C. for 12 h. TLC (Petroleum ether/Ethyl acetate=3/1, R_{f}=0.59) indicated starting material was consumed completely and many new spots formed. After 12 h, the reaction mixture was quenched by H₂O (10 mL) at 20° C. The mixture was diluted with H₂O (30 mL), and extracted by EtOAc (50 mL*3). Then organic phase was combined and washed by brine (30 mL*2), dried with Na₂SO₄, filtered. The organic phase was concentrated under reduced pressure at 40° C. to give a residue. The residue was purified by column chromatography (SiO₂, Petroleum ether/Ethyl acetate=50/1 to 3/1) to provide product (2.2 g, crude) as a white solid.

¹H NMR (400 MHz, DMSO-d₆) δ=7.67-7.61 (m, 2H), 6.07-5.93 (m, 1H), 5.38 (dd, J=1.2, 17.2 Hz, 1H), 5.31 (dd, J=0.8, 10.8 Hz, 1H), 4.93-4.83 (m, 2H), 3.91 (s, 3H)

### Preparation of Intermediate Methyl 3-(allyloxy)-4-nitro-5-((thiazol-5-ylmethyl)amino)benzoate (3)

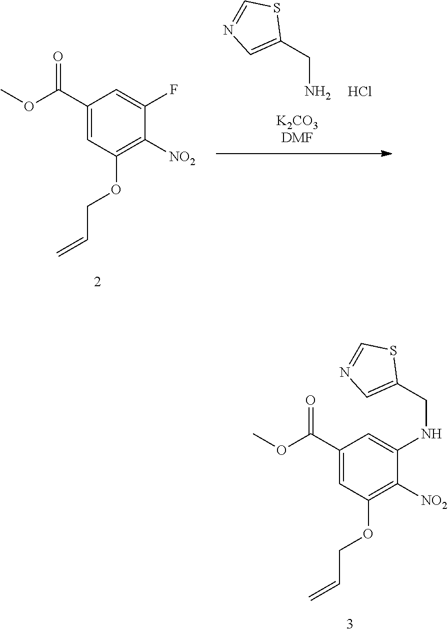 [SMILES: C=CCOc1cc(C(=O)OC)cc(F)c1[N+](=O)[O-] ; NCc1cncs1 ; Cl ; C=CCOc1cc(C(=O)OC)cc(NCc2cncs2)c1[N+](=O)[O-]]

To a solution of methyl 3-(allyloxy)-5-fluoro-4-nitrobenzoate 2 (1.9 g, 7.45 mmol, 1 eq) in DMF (20 mL) was added K₂CO₃ (3.09 g, 22.3 mmol, 3 eq) and thiazol-5-ylmethanamine (2.09 g, 11.2 mmol, 1.5 eq, 2HCl). The mixture was stirred at 60° C. for 12 h. TLC (Petroleum ether/Ethyl acetate=1/1, R_{f}=0.29) indicated starting material was consumed completely. The reaction mixture was quenched by addition H₂O (40 mL) at 25° C., and then extracted with EtOAc (15 mL*2). Then organic phase was combined and washed by H₂O (20 mL*2), dried with Na₂SO₄, filtered. The organic phase was concentrated under reduced pressure at 40° C. to give a residue. The residue was purified by column chromatography (SiO₂, Petroleum ether/Ethyl acetate-50/1 to 1/1) to provide product (810 mg, 2.32 mmol, 31% yield) as a white solid.

¹H NMR (400 MHz, DMSO-d₆) δ=8.96 (s, 1H), 7.82 (s, 1H), 7.06 (d, J=1.2 Hz, 1H), 6.98 (br t, J=6.0 Hz, 1H), 6.92 (d, J=1.2 Hz, 1H), 5.97-5.93 (m, 1H), 5.41-5.30 (m, 1H), 5.27-5.23 (m, 1H), 4.69-4.66 (m, 4H), 3.83 (s, 3H)

### Preparation of Intermediate Methyl 3-(allyloxy)-4-amino-5-((thiazol-5-ylmethyl)amino)benzoate (4)

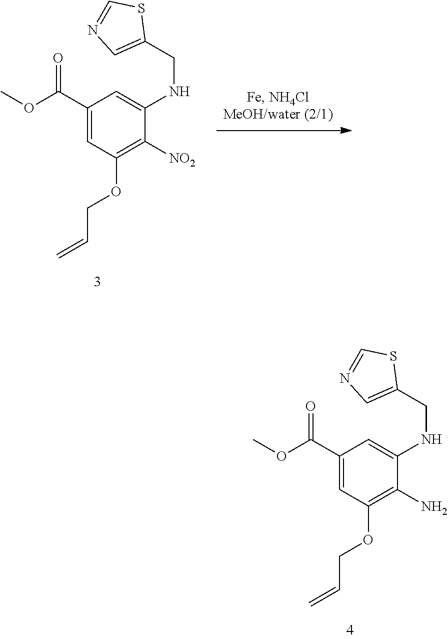 [SMILES: C=CCOc1cc(C(=O)OC)cc(NCc2cncs2)c1[N+](=O)[O-] ; C=CCOc1cc(C(=O)OC)cc(NCc2cncs2)c1N]

Equip a 25 mL three-necked round bottom flask and thermometer, N₂ balloon. MeOH (8 mL) and H₂O (4 mL) was charged to the 25 mL three-necked round bottom flask, and then methyl 3-(allyloxy)-4-nitro-5-((thiazol-5-ylmethyl)amino)benzoate 3 (800 mg, 2.29 mmol, 1 eq) and NH₄Cl (612.45 mg, 11.45 mmol, 5 eq) was added to the mixture at 20° C. At 20° C., Fe (639 mg, 11.5 mmol, 5 eq) was added portionwise to the reaction mixture at 20° C. After the addition, the mixture was stirred at 70° C. for 1 h. LC-MS showed starting material was consumed completely and desired mass was detected. The suspension was filtered through a pad of Celite and the filter cake was washed with MeOH (10 mL*2). The combined filtrates were concentrated to dryness to give product. The residue was purified by column chromatography (SiO₂, Petroleum ether/Ethyl acetate=50/1 to 1/1) to provide product (450 mg, 1.41 mmol, 61% yield) as a white solid.

LCMS: RT=0.656 min, MS cal.: 319.1, [M+H]⁺=320.2

¹H NMR (400 MHz, DMSO-d₆) δ=8.95 (s, 1H), 7.84 (s, 1H), 6.95 (s, 1H), 6.92 (s, 1H), 6.18-6.00 (m, 1H), 5.50 (br t, J=5.6 Hz, 1H), 5.43 (d, J=17.2 Hz, 1H), 5.25 (d, J=10.8 Hz, 1H), 5.01 (s, 2H), 4.63-4.53 (m, 4H), 3.72 (s, 3H)

### Preparation of Intermediate Methyl 4-(allyloxy)-2-(chloromethyl)-1-(thiazol-5-ylmethyl)-1H-benzo[d]imidazole-6-carboxylate (5)

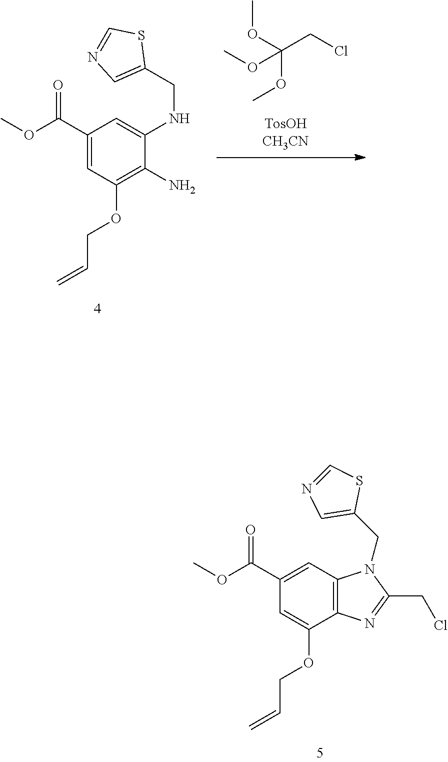

To a solution of methyl 3-(allyloxy)-4-amino-5-((thiazol-5-ylmethyl)amino)benzoate 4 (450 mg, 1.41 mmol, 1 eq) in ACN (5 mL) was added 2-chloro-1,1,1-trimethoxyethane (653 mg, 4.23 mmol, 570 μL, 3 eq) and 4-methylbenzenesulfonic acid (48.5 mg, 282 μmol, 0.2 eq). The mixture was stirred at 60° C. for 12 h. LC-MS showed starting material was consumed completely and desired mass was detected. The reaction mixture was concentrated under reduced pressure to provide product (532 mg, 1.41 mmol, 99% yield) as a white solid.

LCMS: RT=0.686 min, MS cal.: 377.1, 379.1 [M+H]⁺=378.1

¹H NMR (400 MHz, DMSO-d₆) δ=9.02 (s, 1H), 8.01 (s, 1H), 7.90 (s, 1H), 7.33 (s, 1H), 6.15-6.08 (m, 1H), 5.97 (s, 2H), 5.46 (dd, J=1.6, 17.2 Hz, 1H), 5.30 (dd, J=1.6 10.8 Hz, 1H), 5.17 (s, 2H), 4.83 (d, J=5.2 Hz, 2H), 3.89-3.85 (m, 3H)

### Preparation of Intermediate Methyl 4-(allyloxy)-2-((4-(6-((4-cyano-2-fluorobenzyl)oxy)pyridin-2-yl)piperidin-1-yl)methyl)-1-(thiazol-5-ylmethyl)-1H-benzo[d]imidazole-6-carboxylate (6)

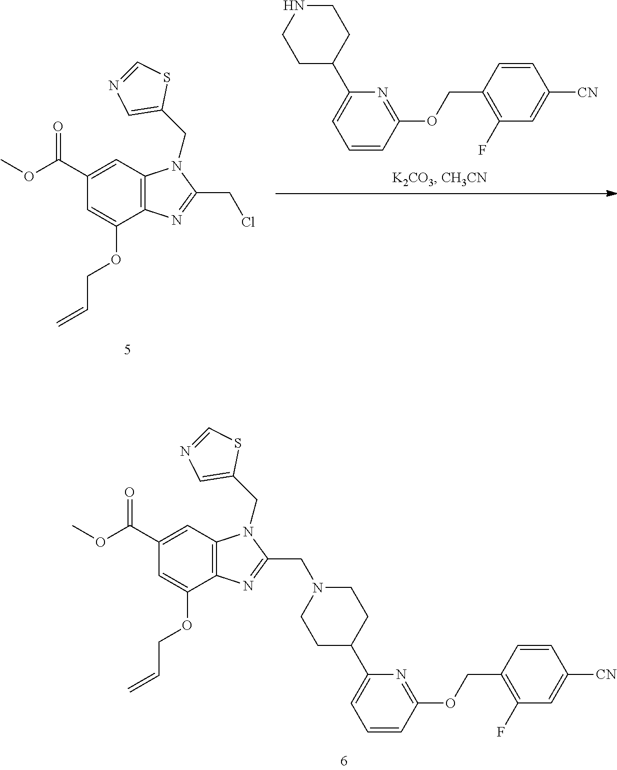 [SMILES: C=CCOc1cc(C(=O)OC)cc2c1nc(CCl)n2Cc1cncs1 ; N#Cc1ccc(COc2cccc(C3CCNCC3)n2)c(F)c1 ; C=CCOc1cc(C(=O)OC)cc2c1nc(CN1CCC(c3cccc(OCc4ccc(C#N)cc4F)n3)CC1)n2Cc1cncs1]

To a solution of methyl 4-(allyloxy)-2-(chloromethyl)-1-(thiazol-5-ylmethyl)-1H-benzo[d]imidazole-6-carboxylate 5 (232 mg, 614 μmol, 1 eq) in ACN (2.3 mL) was added K₂CO₃ (255 mg, 1.84 mmol, 3 eq) and 3-fluoro-4-(((6-(piperidin-4-yl)pyridin-2-yl)oxy)methyl)benzonitrile (210 mg, 675 μmol, 1.1 eq). The mixture was stirred at 60° C. for 12 h. LC-MS showed starting material was consumed completely and desired mass was detected. The mixture was diluted with H₂O (5 mL), and extracted by EtOAc (10 mL*2). Then organic phase was combined and washed by H₂O (8 mL), dried with Na₂SO₄, filtered. The organic phase was concentrated under reduced pressure at 40° C. to give a residue. The residue was purified by column chromatography (SiO₂, Petroleum ether/Ethyl acetate=50/1 to 0/1) to provide product (300 mg, 460 μmol, 74% yield) as a white solid.

LCMS: RT=0.737 min, MS cal.: 652.2, [M+H]⁺=653.2

¹H NMR (400 MHz, DMSO-d₆) δ=8.97 (s, 1H), 8.00 (s, 1H), 7.90-7.83 (m, 2H), 7.69-7.66 (m, 2H), 7.64 (t, J=7.6 Hz, 1H), 7.29 (s, 1H), 6.85 (d, J=7.6 Hz, 1H), 6.71 (d, J=8.0 Hz, 1H), 6.19-6.07 (m, 1H), 5.95 (s, 2H), 5.50-5.41 (m, 3H), 5.30 (d, J=10.8 Hz, 1H), 4.82 (d, J=5.2 Hz, 2H), 3.85 (m, 5H), 2.95-2.92 (m, 2H), 2.61-2.53 (m, 1H), 2.19-2.16 (m, 2H), 1.74-1.62 (m, 4H).

### Preparation of Intermediate Methyl 2-((4-(6-((4-cyano-2-fluorobenzyl)oxy)pyridin-2-yl)piperidin-1-yl)methyl)-4-hydroxy-1-(thiazol-5-ylmethyl)-1H-benzo[d]imidazole-6-carboxylate (7)

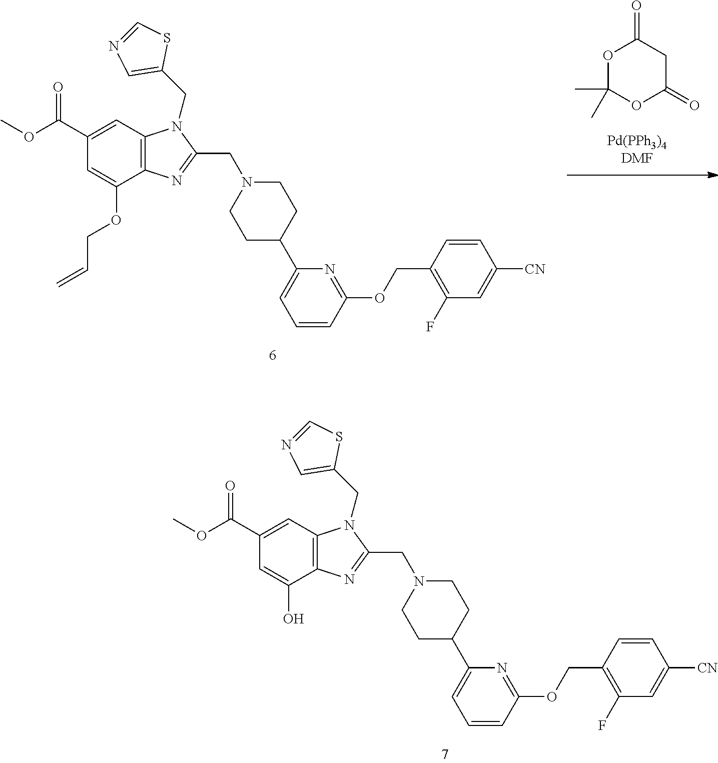

To a solution of methyl 4-(allyloxy)-2-((4-(6-((4-cyano-2-fluorobenzyl)oxy)pyridin-2-yl)piperidin-1-yl)methyl)-1-(thiazol-5-ylmethyl)-1H-benzo[d]imidazole-6-carboxylate 6 (300 mg, 460 μmol, 1 eq) in DMF (3 mL) was added 2,2-dimethyl-1,3-dioxane-4,6-dione (331 mg, 2.30 mmol, 5 eq) and Pd(PPh₃)₄ (53 mg, 46 μmol, 0.1 eq). The mixture was stirred at 25° C. for 12 h. LC-MS showed starting material was consumed completely and desired mass was detected. The mixture was diluted with H₂O (5 mL), and extracted by EtOAc (5 mL*2). Then organic phase was combined and washed by H₂O (5 mL), dried with Na₂SO₄, filtered. The organic phase was concentrated under reduced pressure at 40° C. to give a residue. The residue was purified by column chromatography (SiO₂, Petroleum ether/Ethyl acetate=20/1 to 0/1) to provide product (150 mg, 245 μmol, 53% yield) as a white solid.

LCMS: RT=0.679 min, MS cal.: 612.2, [M+H]⁺=613.2

### Preparation of 2-((4-(6-((4-Cyano-2-fluorobenzyl)oxy)pyridin-2-yl)piperidin-1-yl)methyl)-4-hydroxy-1-(thiazol-5-ylmethyl)-1H-benzo[d]imidazole-6-carboxylic acid, Compound 82 (8)

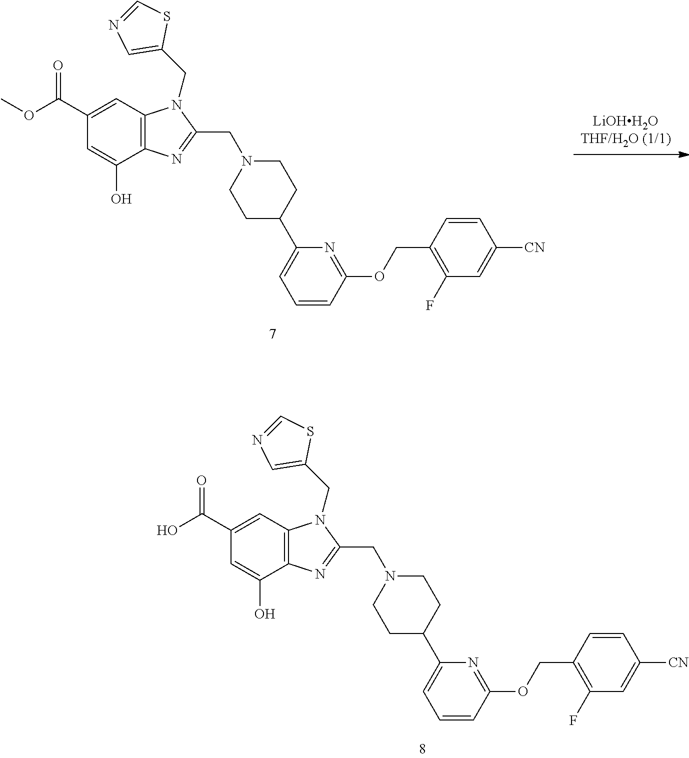 [SMILES: COC(=O)c1cc(O)c2nc(CN3CCC(c4cccc(OCc5ccc(C#N)cc5F)n4)CC3)n(Cc3cncs3)c2c1 ; N#Cc1ccc(COc2cccc(C3CCN(Cc4nc5c(O)cc(C(=O)O)cc5n4Cc4cncs4)CC3)n2)c(F)c1]

To a solution of methyl 2-((4-(6-((4-cyano-2-fluorobenzyl)oxy)pyridin-2-yl)piperidin-1-yl)methyl)-4-hydroxy-1-(thiazol-5-ylmethyl)-1H-benzo[d]imidazole-6-carboxylate, 7 (150 mg, 245 μmol, 1 eq) in H₂O (0.75 mL) and THF (0.75 mL) was added LiOH·H₂O (20.6 mg, 490 μmol, 2 eq). The mixture was stirred at 25° C. for 12 h. LC-MS showed ˜18% of starting material remained. Several new peaks were shown on LC-MS and ˜60% of desired compound was detected. The mixture was concentrated under reduced pressure to give a residue. The crude product was purified by reversed-phase HPLC (column: Waters Xbridge Prep OBD C18 150*40 mm*10 um; mobile phase: [H₂O (10 mM NH₄HCO₃)-ACN]; gradient: 20%-70% B over 8.0 min) to provide product (30.5 mg, 51.0 μmol, 20% yield) as a white solid.

LCMS: RT=2.571 min, MS cal.: 598.2, [M+H]⁺=599.2

HPLC: RT=9.243 min, purity: 98%

¹H NMR (400 MHz, DMSO-d₆) δ=8.96 (s, 1H), 7.99 (s, 1H), 7.88 (d, J=10.0 Hz, 1H), 7.72-7.68 (m, 2H), 7.64 (t, J=7.6 Hz, 1H), 7.59 (s, 1H), 7.19 (d, J=1.2 Hz, 1H), 6.86 (d, J=7.4 Hz, 1H), 6.71 (d, J=8.0 Hz, 1H), 5.87 (s, 2H), 5.47 (s, 2H), 3.83 (s, 2H), 2.97-2.94 (m, 2H), 2.62-2.53 (m, 1H), 2.20-2.15 (m, 2H), 1.75-1.63 (m, 4H).

### Example A23. —Preparation of Compound 83. (S)-2-((4-(2-(5-Chloropyridin-2-yl)-2-methylbenzo[d][1,3]dioxol-4-yl)piperidin-1-yl)methyl)-4-(difluoromethoxy)-1-methyl-1H-benzo[d]imidazole-6-carboxylic acid (Compound 83) (7)

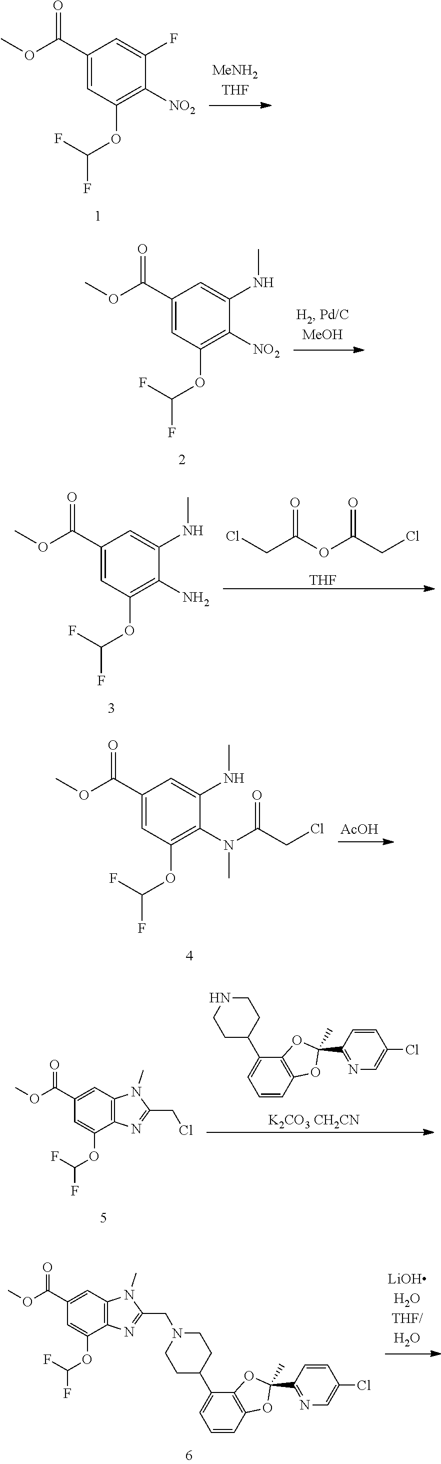

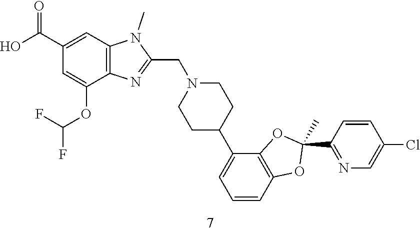 [SMILES: Cn1c(CN2CCC(c3cccc4c3O[C@@](C)(c3ccc(Cl)cn3)O4)CC2)nc2c(OC(F)F)cc(C(=O)O)cc21]

### Preparation of Intermediate Methyl 3-(difluoromethoxy)-5-(methylamino)-4-nitrobenzoate (2)

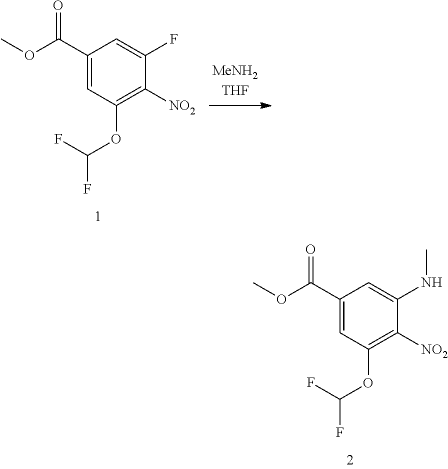 [SMILES: COC(=O)c1cc(F)c([N+](=O)[O-])c(OC(F)F)c1 ; CNc1cc(C(=O)OC)cc(OC(F)F)c1[N+](=O)[O-]]

THF (6 mL) was charged to a 50 mL three-necked round bottom flask, then methyl 3-(difluoromethoxy)-5-fluoro-4-nitrobenzoate 1 (600 mg, 2.26 mmol, 1 eq) was added to the mixture at 25° C. for 1 min. At 25° C. (inner temperature), CH₃NH₂ (2 M, 2.26 mL, 2 eq) was added dropwise to the reaction mixture at 25° C. within 1 min. After the addition, the mixture was stirred at 25° C. for 4 h. TLC indicated ˜10% of starting material remained, and one major new spot with larger polarity was detected (R_{f}=0.57). After 12 h, the reaction mixture was added FA (1 M) to adjust pH=5 at 25° C. The mixture was extracted by EtOAc (30 mL*3). Then the organic phase was combined and washed by H₂O 20 mL, brine 20 mL, dried with Na₂SO₄, filtered. The organic phase was concentrated under reduced pressure at 40° C. to give a residue. The residue was purified by column chromatography (SiO₂, Petroleum ether/Ethyl acetate=1/0 to 95/5) to provide product (460 mg, 1.67 mmol, 73% yield) as an orange solid.

LCMS: RT=0.777 min, MS cal.: 276.1, [M−H]⁻=275.1

¹H NMR (400 MHz, DMSO-d6) δ=7.40 (t, J=72.4 Hz, 1H), 7.20 (s, 1H), 7.03 (s, 1H), 6.76 (br q, J=4.4 Hz, 1H), 3.89 (s, 3H), 2.82 (d, J=4.8 Hz, 3H)

### Preparation of Intermediate Methyl 4-amino-3-(difluoromethoxy)-5-(methylamino)benzoate (3)

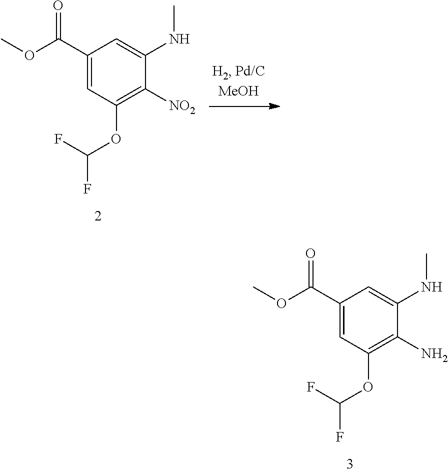

To a solution of Pd/C (100 mg, 10%) in MeOH (10 mL) was added methyl 3-(difluoromethoxy)-5-(methylamino)-4-nitrobenzoate 2 (400 mg, 1.45 mmol, 1 eq) under N₂ atmosphere. The suspension was degassed and purged with H₂ for 3 times. The mixture was stirred under H₂ (15 Psi) at 20° C. for 2 h. LC-MS showed starting material was consumed completely and one main peak with desired mass was detected. After 2 h, the suspension was filtered through a pad of celite and filter cake was washed with MeOH (20 mL×3). The combined filtrates were concentrated to dryness to provide product (350 mg, 1.42 mmol, 98% yield) as a white solid.

¹H NMR (400 MHz, DMSO-d6) δ=7.08 (s, 1H), 7.05 (t, J=74.4 Hz, 1H), 6.86 (s, 1H), 5.25 (br s, 2H), 5.17 (br q, J=5.2 Hz, 1H), 3.76 (s, 3H), 2.76 (d, J=5.2 Hz, 3H)

### Preparation of Intermediate Methyl 4-(2-chloro-N-methylacetamido)-3-(difluoromethoxy)-5-(methylamino)benzoate (4)

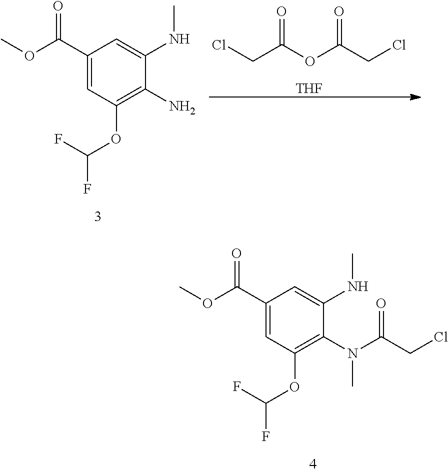

To a solution of methyl 4-amino-3-(difluoromethoxy)-5-(methylamino)benzoate 3 (0.2 g, 812 μmol in THF (5 mL) was added 2-chloroacetic anhydride (139 mg, 812 μmol, 1 eq). The mixture was stirred at 0° C. for 1 h. LC-MS showed starting material was consumed completely and one main peak with desired mass was detected. The reaction mixture was concentrated under reduced pressure to remove solvent to provide product (270 mg, crude) as a yellow solid.

LCMS: RT=0.680 min, MS cal.: 322.1, 324.1, [M−H]⁻=321.1

### Preparation of Intermediate Methyl 2-(chloromethyl)-4-(difluoromethoxy)-1-methyl-1H-benzo[d]imidazole-6-carboxylate (5)

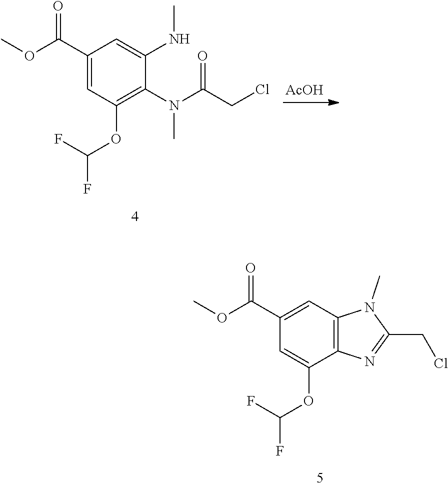 [SMILES: "AcOH" ; CNc1cc(C(=O)OC)cc(OC(F)F)c1N(C)C(=O)CCl ; COC(=O)c1cc(OC(F)F)c2nc(CCl)n(C)c2c1]

A mixture of methyl 4-(2-chloro-N-methylacetamido)-3-(difluoromethoxy)-5-(methylamino)benzoate 4 (270 mg, 837 μmol, 1 eq) in AcOH (5 mL) was degassed and purged with N₂ for 3 times, and then the mixture was stirred at 80° C. for 12 h under N₂ atmosphere. LC-MS showed starting material was consumed completely and one main peak with desired mass was detected. The reaction mixture was concentrated under reduced pressure to provide product (265 mg, crude) as a yellow oil.

LCMS: RT=0.680 min, MS cal.: 304.0, 306.0, [M+H]⁺=305.1

### Preparation of Intermediate Methyl (S)-2-((4-(2-(5-chloropyridin-2-yl)-2-methylbenzo[d][1,3]dioxol-4-yl)piperidin-1-yl)methyl)-4-(difluoromethoxy)-1-methyl-1H-benzo[d]imidazole-6-carboxylate (6)

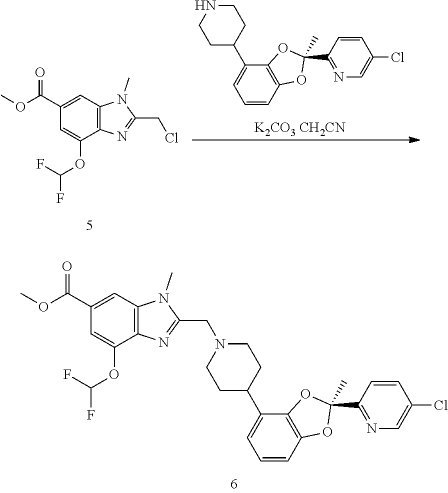 [SMILES: COC(=O)c1cc(OC(F)F)c2nc(CCl)n(C)c2c1 ; "K2CO3 CH2CN" ; C[C@]1(c2ccc(Cl)cn2)Oc2cccc(C3CCNCC3)c2O1 ; COC(=O)c1cc(OC(F)F)c2nc(CN3CCC(c4cccc5c4O[C@@](C)(c4ccc(Cl)cn4)O5)CC3)n(C)c2c1]

To a solution of methyl 2-(chloromethyl)-4-(difluoromethoxy)-1-methyl-1H-benzo[d]imidazole-6-carboxylate 5 (265 mg, 870 μmol, 1 eq) in ACN (3 mL) was added K₂CO₃ (361 mg, 2.61 mmol, 3 eq) and (S)-5-chloro-2-(2-methyl-4-(piperidin-4-yl)benzo[d][1,3]dioxol-2-yl)pyridine (317 mg, 957 μmol, 1.1 eq). The mixture was stirred at 60° C. for 4 h. LC-MS showed starting material was consumed completely and one main peak with desired mass was detected. The reaction mixture was diluted with H₂O 30 mL and extracted with EtOAc (20 mL*3). The combined organic layers were washed with brine 20 mL, dried with Na₂SO₄, filtered, and concentrated under reduced pressure to give a residue. The residue was purified by column chromatography (SiO₂, Petroleum ether/Ethyl acetate=95/5 to 1/1) to provide product (370 mg, 618 μmol, 71% yield) as a yellow solid.

LCMS: RT=0.974 min, MS cal.: 598.2, 599.2, [M+H]⁺=599.3

¹H NMR (400 MHz, CHLOROFORM-d) δ=8.62 (d, J=2.0 Hz, 1H), 7.98 (s, 1H), 7.73-7.66 (m, 2H), 7.57 (d, J=8.4 Hz, 1H), 7.32 (t, J=74.4 Hz, 1H), 6.82-6.76 (m, 1H), 6.74-6.69 (m, 2H), 3.99 (s, 3H), 3.97 (s, 3H), 3.90 (s, 2H), 3.06-2.98 (m, 2H), 2.78-2.76 (m, 1H), 2.34-2.32 (m, 2H), 2.05 (s, 3H), 1.94-1.76 (m, 4H)

### Preparation of Compound 83 (S)-2-((4-(2-(5-Chloropyridin-2-yl)-2-methylbenzo[d][1,3]dioxol-4-yl)piperidin-1-yl)methyl)-4-(difluoromethoxy)-1-methyl-1H-benzo[d]imidazole-6-carboxylic acid (7)

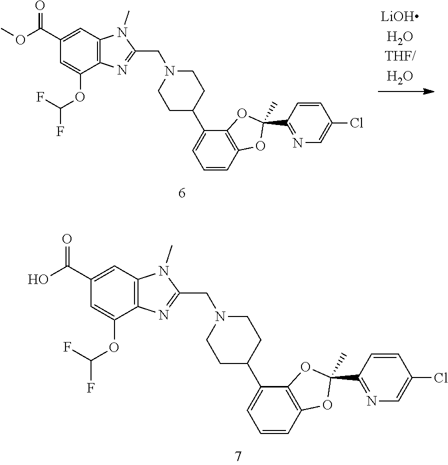 [SMILES: COC(=O)c1cc(OC(F)F)c2nc(CN3CCC(c4cccc5c4O[C@@](C)(c4ccc(Cl)cn4)O5)CC3)n(C)c2c1 ; Cn1c(CN2CCC(c3cccc4c3O[C@@](C)(c3ccc(Cl)cn3)O4)CC2)nc2c(OC(F)F)cc(C(=O)O)cc21]

To a solution of methyl (S)-2-((4-(2-(5-chloropyridin-2-yl)-2-methylbenzo[d][1,3]dioxol-4-yl)piperidin-1-yl)methyl)-4-(difluoromethoxy)-1-methyl-1H-benzo[d]imidazole-6-carboxylate 6 (100 mg, 167 μmol, 1 eq) in THF (0.7 mL) and H₂O (0.3 mL) was added LiOH·H₂O (10.5 mg, 250 μmol, 1.5 eq). The mixture was stirred at 25° C. for 12 hr. LC-MS showed 6 was consumed completely and one main peak with desired mass was detected. The reaction mixture was filtered and the filtrate was purified by prep-HPLC (column: Waters Xbridge Prep OBD C18 150*40 mm*10 um; mobile phase: [H₂O (10 mM NH₄HCO₃)-ACN]; gradient: 15%-65% B over 8.0 min) to provide product (28.9 mg, 49.2 μmol, 29% yield, 99% purity) as a white solid.

LCMS: RT=2.698 min, MS cal.: 584.2, 586.2, [M+H]⁺=585.2

HPLC: RT=6.696 min

¹H NMR (400 MHz, METHANOL-d4) δ=8.60 (s, 1H), 8.10 (s, 1H), 7.87 (dd, J=2.4, 8.4 Hz, 1H), 7.68 (s, 1H), 7.65 (dd, J=0.4, 8.4 Hz, 1H), 7.26 (t, J=74.4 Hz, 1H), 6.81-6.76 (m, 1H), 6.75-6.68 (m, 2H), 4.01 (s, 3H), 4.00 (s, 2H), 3.15-3.08 (m, 2H), 2.82-2.72 (m, 1H), 2.42-2.39 (m, 2H), 2.01 (s, 3H), 1.97-1.80 (m, 4H).

### Example A24. —Preparation of Compound 84. (S)-2-((4-(2-(5-Chloropyridin-2-yl)-2-methylbenzo[d][1,3]dioxol-4-yl)piperidin-1-yl)methyl)-4-isopropoxy-1-methyl-1H-benzo[d]imidazole-6-carboxylic acid (Compound 84) (6)

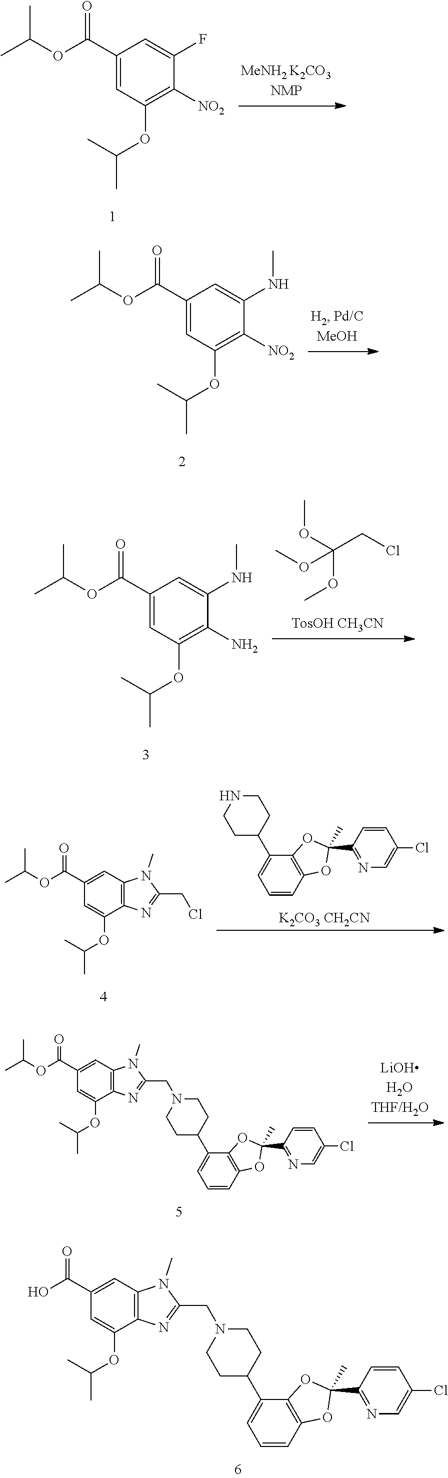

### Preparation of Intermediate Isopropyl 3-isopropoxy-5-(methylamino)-4-nitrobenzoate (2)

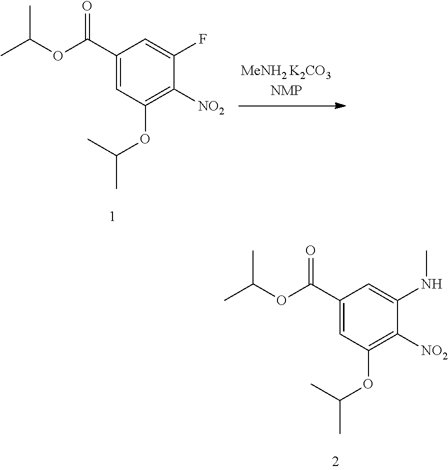 [SMILES: CC(C)OC(=O)c1cc(F)c([N+](=O)[O-])c(OC(C)C)c1 ; CNc1cc(C(=O)OC(C)C)cc(OC(C)C)c1[N+](=O)[O-]]

NMP (5 mL) was charged to the three-necked round bottom flask, then isopropyl 3-fluoro-5-isopropoxy-4-nitrobenzoate 1 (500 mg, 1.75 mmol, 1 eq) and K₂CO₃ (1.21 g, 8.76 mmol, 5 eq) was added to the mixture at 25° C. for 1 min. At 25° C. (inner temperature), MeNH₂ (2 M, 1.75 mL, 2 eq) was added dropwise to the reaction mixture at 25° C. within 1 min. After the addition, the mixture was stirred at 25° C. for 12 h. TLC indicated starting material was consumed completely and one new spot formed. After 12 h, the reaction mixture was added FA (1 M) to pH=5 at 20° C. The mixture was extracted with EtOAc 30 mL (10 mL*3). Then organic phase was combined and washed by H₂O 10 mL, brine 10 mL, dried with Na₂SO₄, filtered. The organic phase was concentrated under reduced pressure at 40° C. to give a residue. The residue was purified by column chromatography (SiO₂, Petroleum ether/Ethyl acetate=19/1 to 10/1) to provide product (350 mg, 1.18 mmol, 67% yield) as a yellow oil.

LCMS: RT=0.582 min, MS cal.: 296.1, [M+H]⁺=297.1

¹H NMR (400 MHz, CHLOROFORM-d) δ=7.00 (d, J=1.2 Hz, 1H), 6.94 (d, J=1.2 Hz, 1H), 5.29-5.22 (m, 1H), 4.72-4.64 (m, 1H), 2.95 (s, 3H), 1.39-1.36 (m, 12H).

### Preparation of Intermediate Isopropyl 4-amino-3-isopropoxy-5-(methylamino)benzoate (3)

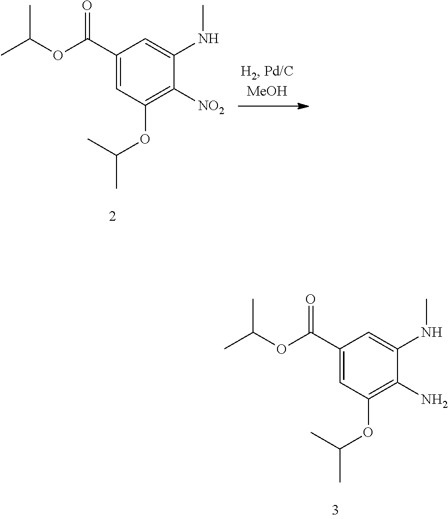 [SMILES: CNc1cc(C(=O)OC(C)C)cc(OC(C)C)c1[N+](=O)[O-] ; CNc1cc(C(=O)OC(C)C)cc(OC(C)C)c1N]

THF (3 mL) was charged to the round bottom flask, then isopropyl 3-isopropoxy-5-(methylamino)-4-nitrobenzoate 2 (300 mg, 1.01 mmol, 1 eq) was added to the mixture at 20° C. for 2 min. At 20° C. (inner temperature), Pd/C (150 mg, 10%) was added to the reaction mixture at 20° C. within 2 min. After the addition, purged with H₂ for 3 times, the mixture was stirred at 20° C. for 1 h. LC-MS showed starting material was consumed completely and desired mass was detected. The reaction mixture was filtered through celite pad. The filter cake was rinsed with MeOH (10 ml*3), and the filtrate was concentrated. The organic phase was concentrated under reduced pressure at 40° C. to give a residue. The reaction mixture was concentrated to provide product (250 mg, 939 μmol, 93% yield) as a yellow oil.

LCMS: RT=0.430 min, MS cal.: 266.2, [M+H]⁺=267.1

¹H NMR (400 MHz, CHLOROFORM-d) δ=7.19 (s, 1H), 7.11 (d, J=1.2 Hz, 1H), 5.27-5.21 (m, 1H), 4.67-4.59 (m, 1H), 2.93 (br s, 3H), 1.40-1.36 (m, 12H).

### Preparation of Intermediate Isopropyl 2-(chloromethyl)-4-isopropoxy-1-methyl-1H-benzo[d]imidazole-6-carboxylate (4)

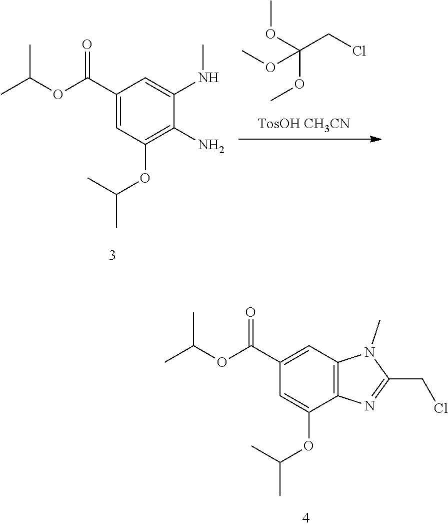

A mixture of isopropyl 4-amino-3-isopropoxy-5-(methylamino)benzoate 3 (250 mg, 939 μmol, 1 eq) in CH₃CN (3 mL) was added TosOH (16.2 mg, 93.9 μmol, 0.1 eq) and 2-chloro-1, 1, 1-trimethoxyethane (174 mg, 1.13 mmol, 152 μL, 1.2 eq). The mixture was stirred at 60° C. for 2h. LC-MS showed starting material was consumed completely and desired mass was detected. The reaction mixture was concentrated under reduced pressure to provide product (300 mg, 924 μmol, 98% yield) as a yellow oil.

LCMS: RT=0.828 min, MS cal.: 324.1, 326.1, [M+H]⁺=325.2

¹H NMR (400 MHz, CHLOROFORM-d) δ=7.54 (d, J=1.2 Hz, 1H), 7.09 (s, 1H), 5.15-5.09 (m, 1H), 4.83 (s, 2H), 4.80-4.75 (m, 1H), 3.77 (s, 3H), 1.32-1.21 (m, 12H).

### Preparation of Intermediate (5) Isopropyl(S)-2-((4-(2-(5-chloropyridin-2-yl)-2-methylbenzo[d][1,3]dioxol-4-yl)piperidin-1-yl)methyl)-4-isopropoxy-1-methyl-1H-benzo[d]imidazole-6-carboxylate

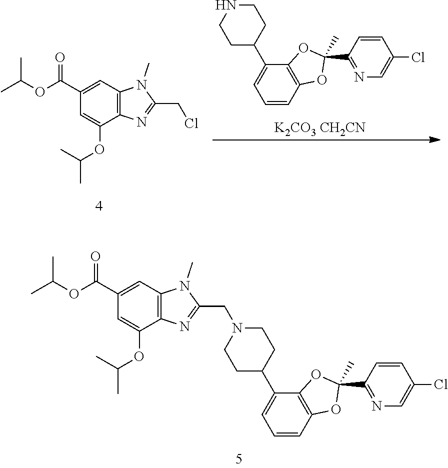

To a solution of isopropyl 2-(chloromethyl)-4-isopropoxy-1-methyl-1H-benzo[d]imidazole-6-carboxylate 4 (300 mg, 924 μmol, 1 eq) in CH₃CN (5 mL) was added K₂CO₃ (383 mg, 2.77 mmol, 3 eq) and(S)-5-chloro-2-(2-methyl-4-(piperidin-4-yl)benzo[d][1,3]dioxol-2-yl)pyridine (306 mg, 924 μmol, 1 eq). The mixture was stirred at 60° C. for 4h. LC-MS showed starting material was consumed completely and desired mass was detected. The residue was diluted with H₂O 20 mL and extracted with EtOAc 30 mL (10 mL*3). The combined organic layers were washed with brine 20 mL, dried over Na₂SO₄ filtered and concentrated under reduced pressure to give a residue. The residue was purified by column chromatography (SiO₂, Petroleum ether/Ethyl acetate=3/1 to 0/1) to provide product (310 mg, 501 μmol, 54% yield) as a yellow solid.

LCMS: RT=1.035 min, MS cal.: 618.3, 619.3, [M+H]⁺=619.4

¹H NMR (400 MHz, CHLOROFORM-d) δ=8.62 (d, J=2.4 Hz, 1H), 7.72-7.65 (m, 2H), 7.57 (d, J=8.4 Hz, 1H), 7.41 (s, 1H), 6.82-6.76 (m, 1H), 6.74-6.68 (m, 2H), 5.36-5.24 (m, 1H), 4.99-4.95 (m, 1H), 4.04-3.84 (m, 5H), 2.95-2.94 (m, 2H), 2.83-2.70 (m, 1H), 2.40-2.22 (m, 2H), 2.05 (s, 3H), 1.92-1.74 (m, 4H), 1.48 (d, J=6.0 Hz, 6H), 1.41 (d, J=6.4 Hz, 6H).

### Preparation of Compound 84, (S)-2-((4-(2-(5-Chloropyridin-2-yl)-2-methylbenzo[d][1,3]dioxol-4-yl)piperidin-1-yl)methyl)-4-isopropoxy-1-methyl-1H-benzo[d]imidazole-6-carboxylic acid (6)

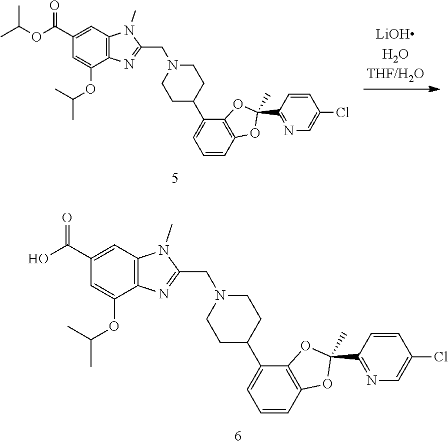 [SMILES: CC(C)OC(=O)c1cc(OC(C)C)c2nc(CN3CCC(c4cccc5c4O[C@@](C)(c4ccc(Cl)cn4)O5)CC3)n(C)c2c1 ; CC(C)Oc1cc(C(=O)O)cc2c1nc(CN1CCC(c3cccc4c3O[C@@](C)(c3ccc(Cl)cn3)O4)CC1)n2C]

To a solution of isopropyl(S)-2-((4-(2-(5-chloropyridin-2-yl)-2-methylbenzo[d][1,3]dioxol-4-yl)piperidin-1-yl)methyl)-4-isopropoxy-1-methyl-1H-benzo[d]imidazole-6-carboxylate 5 (100 mg, 162 μmol, 1 eq) in THF (0.5 mL) and MeOH (0.5 mL) was added LiOH H₂O (13.6 mg, 323 μmol, 2 eq) in H₂O (0.5 mL). The mixture was stirred at 25° C. for 12 h. LC-MS showed starting material was consumed completely and desired mass was detected. The reaction mixture was purified directly. The crude product was purified by prep-HPLC (neutral condition; column: Waters Xbridge Prep OBD C18 150*40 mm*10 um; mobile phase: [H₂O (10 mM NH₄HCO₃)-ACN]; gradient: 15%-65% B over 8.0 min) to provide product (30.2 mg, 52.4 μmol, 32% yield) as a white solid.

LCMS: RT=2.405 min, MS cal.: 576.2, 577.2, [M+H]⁺=577.3

HPLC: RT=2.922 min, purity: 99.6%

¹H NMR (400 MHz, METHANOL-d₄) δ=8.59 (d, J=2.0 Hz, 1H), 7.87 (dd, J=2.4, 8.4 Hz, 1H), 7.82 (d, J=1.2 Hz, 1H), 7.64 (d, J=8.4 Hz, 1H), 7.44 (d, J=0.8 Hz, 1H), 6.81-6.75 (m, 1H), 6.75-6.66 (m, 2H), 4.96-4.89 (m, 1H), 3.96 (s, 3H), 3.93 (s, 2H), 3.12-3.03 (m, 2H), 2.81-2.69 (m, 1H), 2.43-2.32 (m, 2H), 2.01 (s, 3H), 1.95-1.76 (m, 4H), 1.44 (d, J=6.0 Hz, 6H).

### Example A25. —Preparation of Compound 85. (S)-2-((4-(2-(5-Chloropyridin-2-yl)-2-methylbenzo[d][1,3]dioxol-4-yl)piperidin-1-yl)methyl)-4-methoxy-1-methyl-1H-benzo[d]imidazole-6-carboxylic acid (Compound 85) (6)

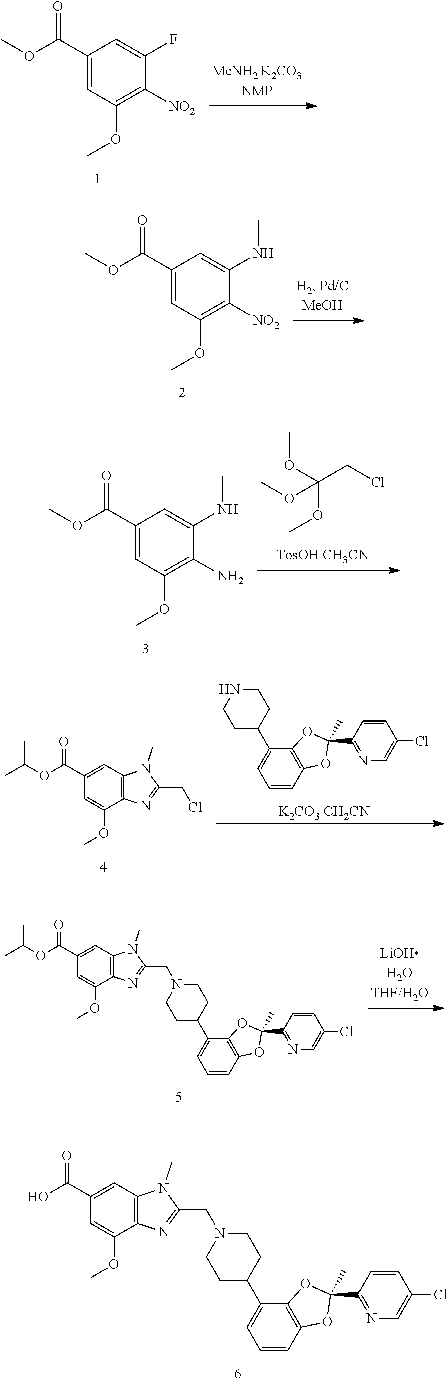

### Preparation of Intermediate Methyl 3-methoxy-5-(methylamino)-4-nitrobenzoate (2)

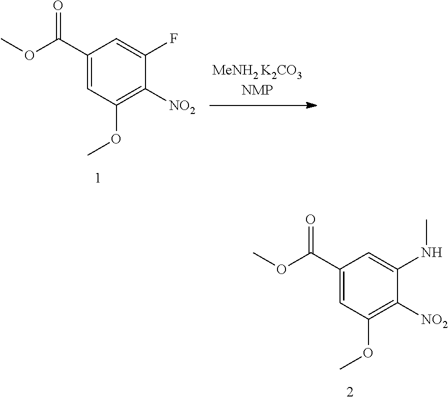 [SMILES: COC(=O)c1cc(F)c([N+](=O)[O-])c(OC)c1 ; CNc1cc(C(=O)OC)cc(OC)c1[N+](=O)[O-]]

Equip a 10 mL round bottom flask, and N₂ balloon. NMP (1 mL) was charged to the 10 mL round bottom flask, methyl 3-fluoro-5-methoxy-4-nitrobenzoate 1 (100 mg, 436 μmol, 1 eq) and K₂CO₃ (302 mg, 2.18 mmol, 5 eq) was added at 25° C. within 2 min. At 25° C. (inner temperature), CH₃NH₂·THF (2 M, 436 μL, 2 eq) was added to the reaction mixture at 25° C. within 2 min. After the addition, the mixture was stirred at 25° C. for 2 h. TLC (Petroleum ether/Ethyl acetate=10/1, R_{f}=0.30) indicated starting material was consumed completely and one new spot formed. The reaction was clean according to TLC. After 2 h, the reaction mixture was added FA (1 M) to adjust pH=5 at 25° C. The mixture was extracted with EtOAc 30 mL (10 mL*3). Then organic phase was combined and washed by H₂O 10 mL, brine 10 mL, dried with Na₂SO₄, filtered. The organic phase was concentrated under reduced pressure at 45° C. to give a residue. The residue was purified by column chromatography (SiO₂, Petroleum ether/Ethyl acetate=100/1 to 3/1) to provide product (100 mg, 416 μmol, 95% yield) as a yellow solid.

LCMS: RT=1.473 min, MS cal.: 240.1, [M+H]⁺=241.1

¹H NMR (400 MHz, CHLOROFORM-d) δ=7.08 (d, J=1.6 Hz, 1H), 6.94 (d, J=1.6 Hz, 1H), 3.95 (s, 3H), 3.94 (s, 3H), 2.97 (s, 3H).

### Preparation of Intermediate Methyl 4-amino-3-methoxy-5-(methylamino)benzoate (3)

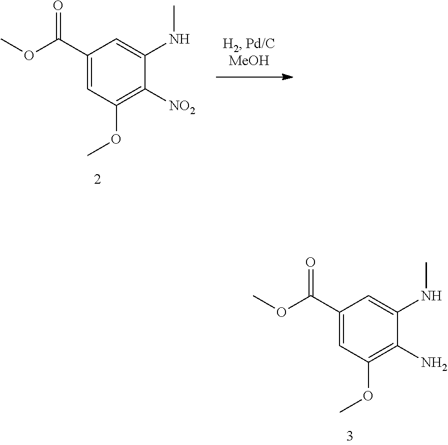

Equip a 50 mL round bottom flask, and H₂ balloon. THF (1 mL) was charged to the 50 mL round bottom flask, methyl 3-methoxy-5-(methylamino)-4-nitrobenzoate 2 (0.1 g, 416 μmol, 1 eq) was added at 25° C. within 2 min. At 25° C. (inner temperature), Pd/C (50 mg, 10%) in THF (1 mL) was added to the reaction mixture at 25° C. within 2 min. After the addition, the mixture was stirred at 25° C. for 2 h. TLC (Petroleum ether/Ethyl acetate=3/1, R_{f}=0.25) indicated starting material was consumed completely and one new spot formed. The reaction was clean according to TLC. After 2 h, the reaction mixture was filtered and the residue was washed with THF 30 mL. The organic phase was concentrated under reduced pressure to provide product (85 mg, 404 μmol, 97% yield) as an orange oil.

LCMS: RT=0.750 min, MS cal.: 210.1, [M+H]⁺=211.1

¹H NMR (400 MHz, CHLOROFORM-d) δ=7.20 (d, J=1.6 Hz, 1H), 7.17 (d, J=1.2 Hz, 1H), 3.91 (s, 3H), 3.89 (s, 3H), 2.93 (s, 3H).

### Preparation of Preparation of Intermediate Methyl 2-(chloromethyl)-4-methoxy-1-methyl-1H-benzo[d]imidazole-6-carboxylate (4)

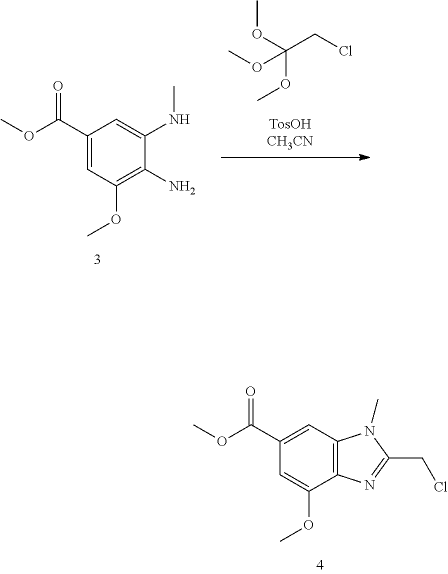

To a solution of methyl 4-amino-3-methoxy-5-(methylamino)benzoate 3 (85 mg, 404 μmol, 1 eq) in ACN (1 mL) was added 2-chloro-1,1,1-trimethoxyethane (93.8 mg, 606 μmol, 81.7 μL, 1.5 eq) and TosOH (6.96 mg, 40.4 μmol, 0.1 eq). The mixture was stirred at 60° C. for 2 h. TLC (Petroleum ether/Ethyl acetate=1/1, R_{f}=0.33) indicated starting material was consumed completely and one new spot formed. The reaction was clean according to TLC. The reaction mixture was concentrated under reduced pressure to provide product (105 mg, 391 μmol, 97% yield) as a white solid.

LCMS: RT=1.133 min, MS cal.: 268.1, 270.1, [M+H]⁺=269.0, 271.0

¹H NMR (400 MHz, CHLOROFORM-d) δ=7.79 (d, J=1.2 Hz, 1H), 7.43 (d, J=0.8 Hz, 1H), 4.93 (s, 2H), 4.07 (s, 3H), 3.98 (s, 3H), 3.94 (s, 3H).

### Preparation of Intermediate Methyl (S)-2-((4-(2-(5-chloropyridin-2-yl)-2-methylbenzo[d][1,3]dioxol-4-yl)piperidin-1-yl)methyl)-4-methoxy-1-methyl-1H-benzo[d]imidazole-6-carboxylate (5)

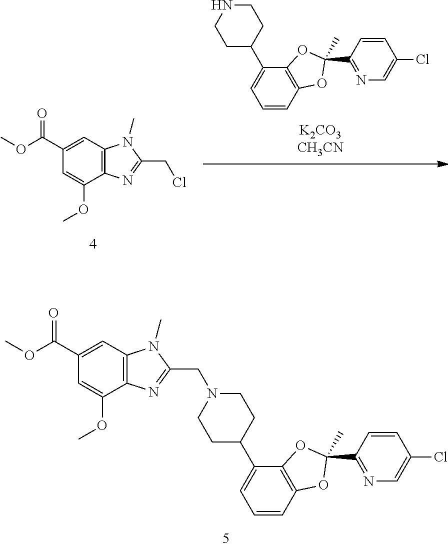 [SMILES: C[C@]1(c2ccc(Cl)cn2)Oc2cccc(C3CCNCC3)c2O1 ; COC(=O)c1cc(OC)c2nc(CCl)n(C)c2c1 ; COC(=O)c1cc(OC)c2nc(CN3CCC(c4cccc5c4O[C@@](C)(c4ccc(Cl)cn4)O5)CC3)n(C)c2c1]

To a solution of methyl 2-(chloromethyl)-4-methoxy-1-methyl-1H-benzo[d]imidazole-6-carboxylate 4 (105 mg, 391 μmol, 1 eq) in ACN (2 mL) was added(S)-5-chloro-2-(2-methyl-4-(piperidin-4-yl)benzo[d][1,3]dioxol-2-yl)pyridine (129 mg, 391 μmol, 1 eq) and K₂CO₃ (162 mg, 1.17 mmol, 3 eq). The mixture was stirred at 60° C. for 2 h. TLC (Petroleum ether/Ethyl acetate=1/1, R_{f}=0.10) indicated starting material was consumed completely and one new spot formed. The reaction was clean according to TLC. The reaction mixture was diluted with H₂O 10 mL and extracted with EtOAc 30 mL (10 mL*3). The organic phase was separated, washed with brine 10 mL, dried over Na₂SO₄, filtered, and concentrated under reduced pressure to give a residue. The residue was purified by column chromatography (SiO₂, Petroleum ether/Ethyl acetate=10/1 to 1/1) to provide product (155 mg, 275 μmol, 70% yield) as a yellow solid.

LCMS: RT=1.479 min, MS cal.: 562.2, 563.2, [M+H]⁺=563.2, 564.2

¹H NMR (400 MHz, CHLOROFORM-d) δ=8.62 (d, J=2.4 Hz, 1H), 7.78 (s, 1H), 7.68 (dd, J=2.4, 8.4 Hz, 1H), 7.57 (d, J=8.4 Hz, 1H), 7.39 (s, 1H), 6.81-6.75 (m, 1H), 6.74-6.67 (m, 2H), 4.07 (s, 3H), 3.97 (s, 6H), 3.95-3.84 (m, 2H), 3.08-2.88 (m, 2H), 2.84-2.70 (m, 1H), 2.42-2.22 (m, 2H), 2.05 (s, 3H), 1.92-1.72 (m, 4H).

### Preparation of (S)-2-((4-(2-(5-Chloropyridin-2-yl)-2-methylbenzo[d][1,3]dioxol-4-yl)piperidin-1-yl)methyl)-4-methoxy-1-methyl-1H-benzo[d]imidazole-6-carboxylic acid, Compound 85 (6)

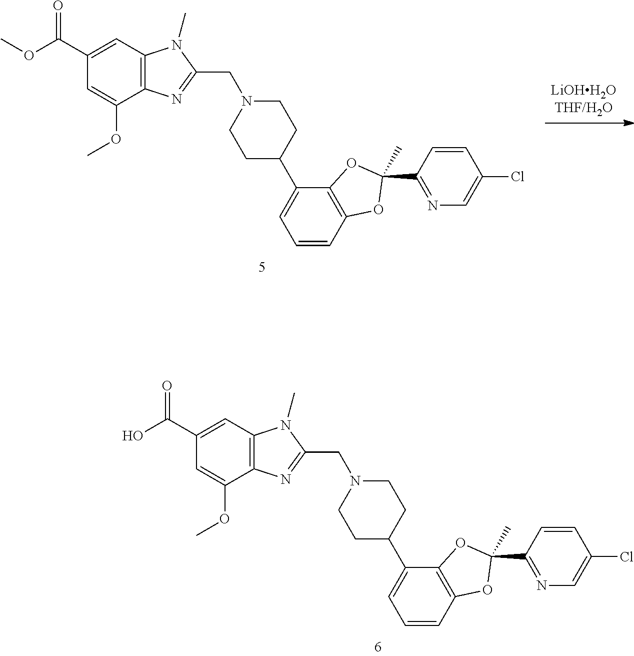 [SMILES: COC(=O)c1cc(OC)c2nc(CN3CCC(c4cccc5c4O[C@@](C)(c4ccc(Cl)cn4)O5)CC3)n(C)c2c1 ; COc1cc(C(=O)O)cc2c1nc(CN1CCC(c3cccc4c3O[C@@](C)(c3ccc(Cl)cn3)O4)CC1)n2C]

To a solution of methyl (S)-2-((4-(2-(5-chloropyridin-2-yl)-2-methylbenzo[d][1,3]dioxol-4-yl)piperidin-1-yl)methyl)-4-methoxy-1-methyl-1H-benzo[d]imidazole-6-carboxylate 5 (140 mg, 249 μmol, 1 eq) in THF (0.7 mL) was added LiOH·H₂O (15.7 mg, 373 μmol, 1.5 eq) in H₂O (0.3 mL). The mixture was stirred at 25° C. for 12 h. LC-MS showed starting material was consumed completely and desired mass was detected. The mixture was purified by prep-HPLC (neutral condition, column: Waters Xbridge BEH C18 100*30 mm*10 um; mobile phase: [H₂O (10 mM NH₄HCO₃)-ACN]; gradient: 20%-60% B over 8.0 min) to provide product (29.2 mg, 53.1 μmol, 21% yield) as a white solid.

LCMS: RT=1.028 min, MS cal.: 548.2, 550.2, [M+H]⁺=549.2, 551.2

LCMS: RT=2.536 min, MS cal.: 548.2, 550.2, [M+H]⁺=549.2, 551.2

HPLC: RT=10.088 min, purity: 99.67%

¹H NMR (400 MHz, METHANOL-d4) δ=8.59 (d, J=2.4 Hz, 1H), 7.89-7.82 (m, 2H), 7.63 (d, J=8.4 Hz, 1H), 7.43 (d, J=1.2 Hz, 1H), 6.80-6.75 (m, 1H), 6.73-6.67 (m, 2H), 4.02 (s, 3H), 3.96 (s, 3H), 3.93 (s, 2H), 3.09-3.06 (m, 2H), 2.80-2.69 (m, 1H), 2.37 (t, J=11.2 Hz, 2H), 2.00 (s, 3H), 1.94-1.79 (m, 4H).

### Example A26. —Preparation of Compound 86. (S)-2-((4-(2-(5-Chloropyridin-2-yl)-2-methylbenzo[d][1,3]dioxol-4-yl)piperidin-1-yl)methyl)-1-isopropyl-4-methoxy-1H-benzo[d]imidazole-6-carboxylic acid (Compound 86) (6)

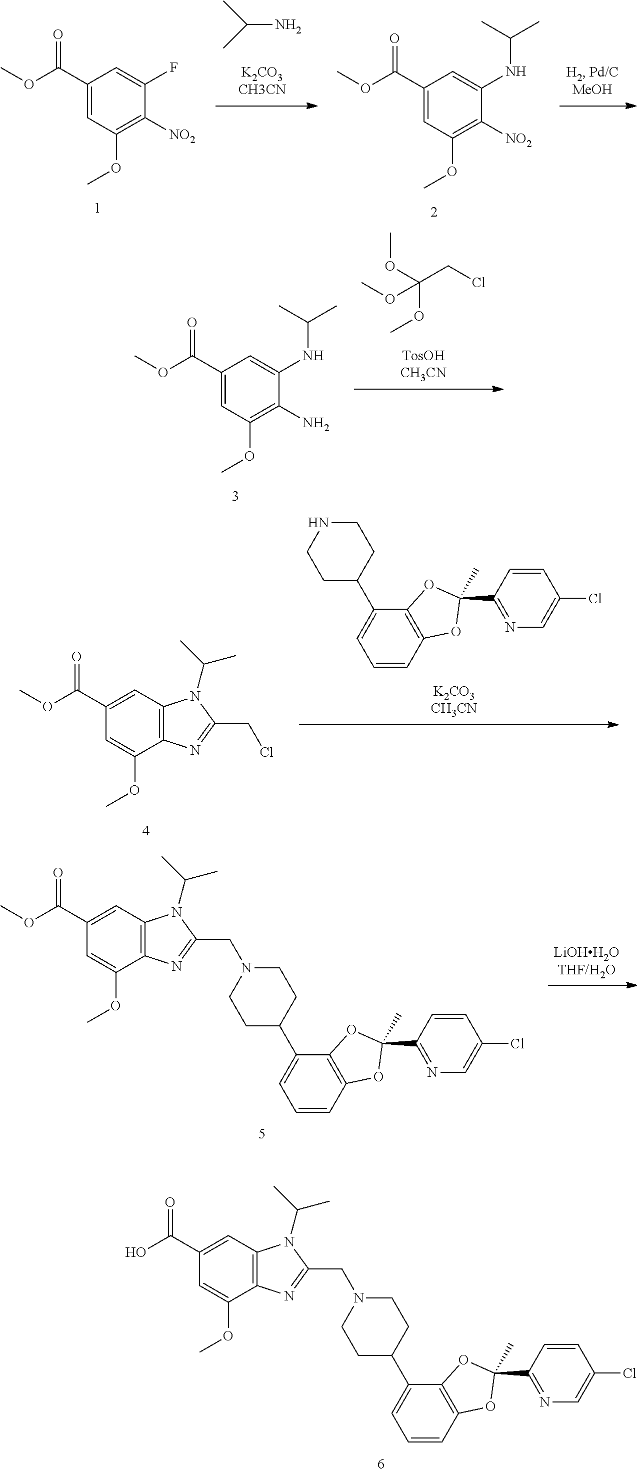

### Preparation of Intermediate Methyl 3-(isopropylamino)-5-methoxy-4-nitrobenzoate (2)

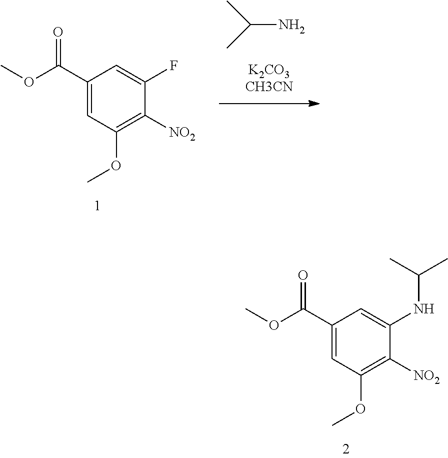

To a solution of methyl 3-fluoro-5-methoxy-4-nitrobenzoate 1 (450 mg, 1.96 mmol, 1 eq) in CH₃CN (5 mL) was added K₂CO₃ (1.36 g, 9.82 mmol, 5 eq) and propan-2-amine (290 mg, 4.91 mmol, 422 μL, 2.5 eq). The mixture was stirred at 60° C. for 12 h. TLC indicated 5% of starting material remained, and one major new spot with larger polarity was detected. The mixture was diluted with H₂O (10 mL), extracted with EtOAc (10 mL*3). The combined organic layer was washed with H₂O 10 mL, brine 10 mL, dried over Na₂SO₄, filtered and the filtrate was concentrated to give a crude product. The residue was purified by column chromatography (SiO₂, PE/EtOAc=1/0 to 30/1) to provide product (0.88 g, 3.28 mmol, 63% yield) as a yellow solid.

LCMS: RT=0.303 min, MS cal.: 268.1, [M+H]⁺=269.0

¹HNMR (400 MHz, CHLOROFORM-d) δ=7.10 (s, 1H), 6.89 (s, 1H), 3.94 (s, 3H), 3.93 (s, 3H), 3.79 (t, J=6.4 Hz, 1H), 1.27 (d, J=6.4 Hz, 6H).

### Preparation of Intermediate Methyl 4-amino-3-(isopropylamino)-5-methoxybenzoate (3)

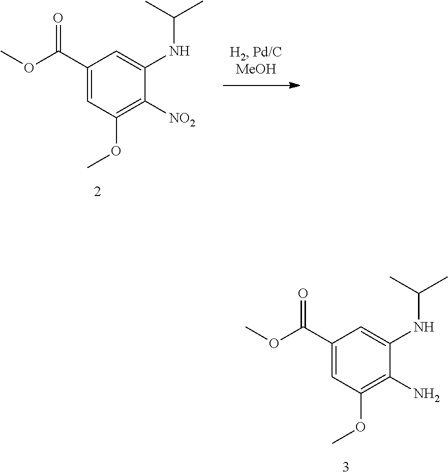 [SMILES: COC(=O)c1cc(NC(C)C)c([N+](=O)[O-])c(OC)c1 ; COC(=O)c1cc(NC(C)C)c(N)c(OC)c1]

To a solution of methyl 3-(isopropylamino)-5-methoxy-4-nitrobenzoate 2 (0.8 g, 2.98 mmol, 1 eq) in THF (8 mL) was added Pd/C (0.5 g, 10%) under N₂ atmosphere. The suspension was degassed and purged with H₂ for 3 times. The mixture was stirred under H₂ (15 psi) at 25° C. for 2 h. LCMS showed starting material was consumed completely and one main peak with desired mass was detected. The reaction mixture was filtered and the filter cake was washed with THE (20 mL). The combined filtrates were concentrated to dryness to provide product (0.61 g, 2.25 mmol, 76% yield) as a white solid.

LCMS: RT=0.297 min, MS cal.: 238.1, [M+H]⁺=239.1

¹HNMR (400 MHz, CHLOROFORM-d) δ=7.15 (s, 2H), 3.92 (s, 3H), 3.88 (s, 3H), 3.67 (t, J=6.0, 1H), 1.24 (d, J=6.0 Hz, 6H).

### Preparation of Intermediate Methyl 2-(chloromethyl)-1-isopropyl-4-methoxy-1H-benzo[d]imidazole-6-carboxylate (4)

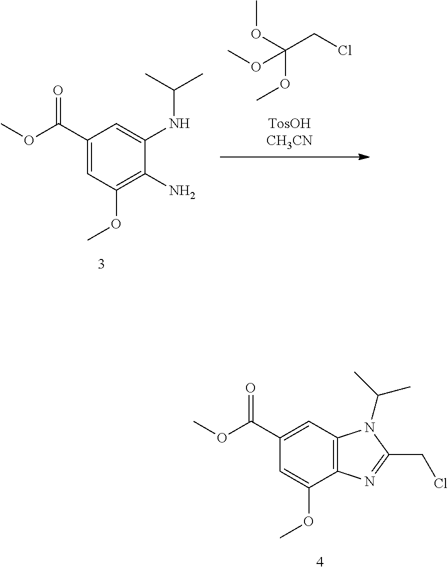

To a solution of methyl 4-amino-3-(isopropylamino)-5-methoxybenzoate 3 (580 mg, 2.43 mmol, 1 eq) in CH₃CN (30 mL) was added 2-chloro-1,1,1-trimethoxyethane (753 mg, 4.87 mmol, 656 μL, 2 eq) and p-TSA (83.8 mg, 487 μmol, 0.2 eq). The mixture was stirred at 60° C. for 2 h. LCMS showed starting material was not consumed completely. The mixture was added 2-chloro-1, 1,1-trimethoxyethane (376 mg, 2.43 mmol, 328 μL, 1 eq) and p-TSA (41.9 mg, 243 μmol, 0.1 eq). The mixture was stirred at 60° C. for another 2h. LCMS showed starting material was consumed completely and one main peak with desired mass was detected. The reaction mixture was diluted with H₂O 20 mL and extracted with EtOAc 60 mL (20 mL*3). The combined organic layers were washed with brine 40 mL (20 mL*2), dried over Na₂SO₄, filtered and concentrated under reduced pressure to provide product (670 mg, 2.05 mmol, 84% yield) as a yellow solid.

LCMS: RT=0.414 min, MS cal.: 296.1/298.1, [M+H]⁺=297.0/299.0

¹HNMR (400 MHz, CHLOROFORM-d) δ=7.94 (s, 1H), 7.39 (s, 1H), 4.88-4.81 (m, 3H), 4.06 (s, 3H), 3.97 (s, 3H), 1.73 (d, J=7.2 Hz, 6H).

### Preparation of Intermediate Methyl (S)-2-((4-(2-(5-Chloropyridin-2-yl)-2-methylbenzo[d][1,3]dioxol-4-yl)piperidin-1-yl)methyl)-1-isopropyl-4-methoxy-1H-benzo[d]imidazole-6-carboxylate (5)

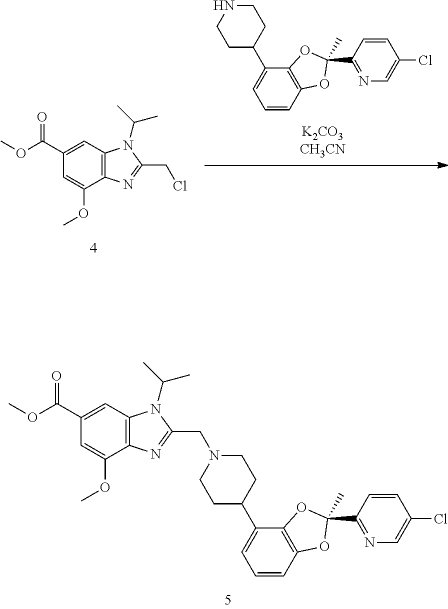 [SMILES: COC(=O)c1cc(OC)c2nc(CCl)n(C(C)C)c2c1 ; C[C@]1(c2ccc(Cl)cn2)Oc2cccc(C3CCNCC3)c2O1 ; COC(=O)c1cc(OC)c2nc(CN3CCC(c4cccc5c4O[C@@](C)(c4ccc(Cl)cn4)O5)CC3)n(C(C)C)c2c1]

To a solution of methyl 2-(chloromethyl)-1-isopropyl-4-methoxy-1H-benzo[d]imidazole-6-carboxylate 4 (200 mg, 674 μmol, 1 eq) in CH₃CN (2 mL) was added K₂CO₃ (279 mg, 2.02 mmol, 3 eq) and(S)-5-chloro-2-(2-methyl-4-(piperidin-4-yl)benzo[d][1,3]dioxol-2-yl)pyridine (334 mg, 1.01 mmol, 1.5 eq). The mixture was stirred at 60° C. for 12 h. LCMS showed starting material was consumed completely and one main peak with desired mass was detected. The reaction mixture was diluted with H₂O 5 mL and extracted with EtOAc 15 mL (5 mL*3). The combined organic layers were washed with brine 10 mL (5 mL*2), dried over Na₂SO₄, filtered, and concentrated under reduced pressure to give a residue. The residue was purified by column chromatography (SiO₂, PE/EtOAc=1/0 to 1/1) to provide product (0.31 g, 514 μmol, 76% yield) as a white solid.

LCMS: RT=0.460 min, MS cal.: 590.2/591.2, [M+H]⁺=591.3/592.3

### Preparation of (S)-2-((4-(2-(5-Chloropyridin-2-yl)-2-methylbenzo[d][1,3]dioxol-4-yl)piperidin-1-yl)methyl)-1-isopropyl-4-methoxy-1H-benzo[d]imidazole-6-carboxylic acid, Compound 86 (6)

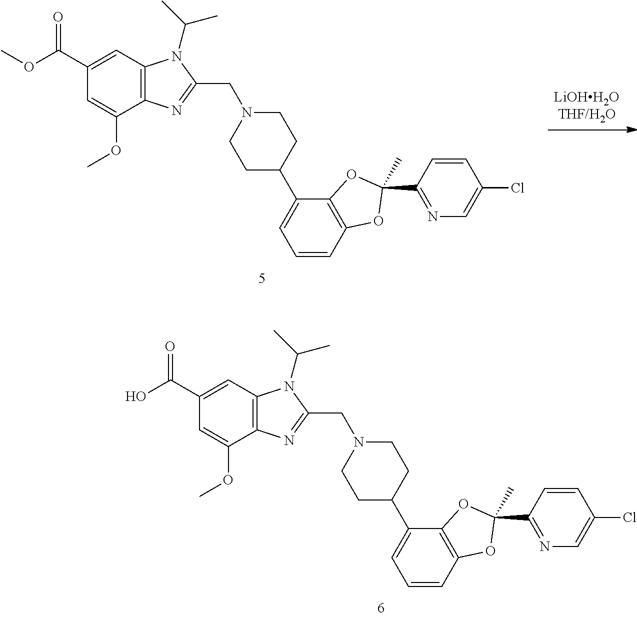 [SMILES: COC(=O)c1cc(OC)c2nc(CN3CCC(c4cccc5c4O[C@@](C)(c4ccc(Cl)cn4)O5)CC3)n(C(C)C)c2c1 ; COc1cc(C(=O)O)cc2c1nc(CN1CCC(c3cccc4c3O[C@@](C)(c3ccc(Cl)cn3)O4)CC1)n2C(C)C]

To a solution of methyl (S)-2-((4-(2-(5-chloropyridin-2-yl)-2-methylbenzo[d][1,3]dioxol-4-yl)piperidin-1-yl)methyl)-1-isopropyl-4-methoxy-1H-benzo[d]imidazole-6-carboxylate 5 (100 mg, 169 μmol, 1 eq) in THF (0.7 mL). After addition, then LiOH·H₂O (7.10 mg, 169 μmol, 1 eq) in H₂O (0.3 mL) was added. The mixture was stirred at 20° C. for 12 h. LCMS showed starting material was not consumed completely. The mixture was added LiOH·H₂O (3.55 mg, 84.6 μmol, 0.5 eq). The mixture was stirred at 20° C. for another 12 hr. LCMS showed starting material was consumed completely and one main peak with desired mass was detected. The mixture was diluted with H₂O 1 mL. The residue was purified by prep-HPLC (basic condition: column: Phenomenex Luna C18 100*30 mm*3 um; mobile phase: [H₂O (0.2% FA)-ACN]; gradient: 20%-60% B over 8.0 min) to provide product (30 mg, 50.4 μmol, 30% yield) as a white solid.

LCMS: RT=0.440 min, MS cal.: 576.2/577.2, [M+H]⁺=577.2/578.2

QC:

LCMS: RT=2.367 min, MS cal.: 576.2/577.2, [M+H]⁺=577.3/578.3

HPLC: RT=10.857 min, purity: 99.41%

¹HNMR (400 MHz, DMSO-d6) δ=8.72 (d, J=2.0 Hz, 1H), 8.00 (dd, J=2.4, 8.4 Hz, 1H), 7.85 (s, 1H), 7.59 (d, J=8.4 Hz, 1H), 7.25 (s, 1H), 6.81-6.75 (m, 2H), 6.75-6.71 (m, 1H), 5.11-5.04 (m, 1H), 3.94 (s, 3H), 3.82 (s, 2H), 2.85-2.3 (m, 2H), 2.70-2.59 (m, 1H), 2.22-2.11 (m, 2H), 2.00 (s, 3H), 1.78-1.67 (m, 4H), 1.59 (d, J=6.8 Hz, 6H).

### Example A27. —Preparation of Compound 32. (S)-2-((4-(2-(5-Chloropyridin-2-yl)-2-methylbenzo[d][1,3]dioxol-4-yl)piperidin-1-yl)methyl)-4-methoxy-1-(thiazol-5-ylmethyl)-1H-benzo[d]imidazole-6-carboxylic acid (Compound 32) (7)

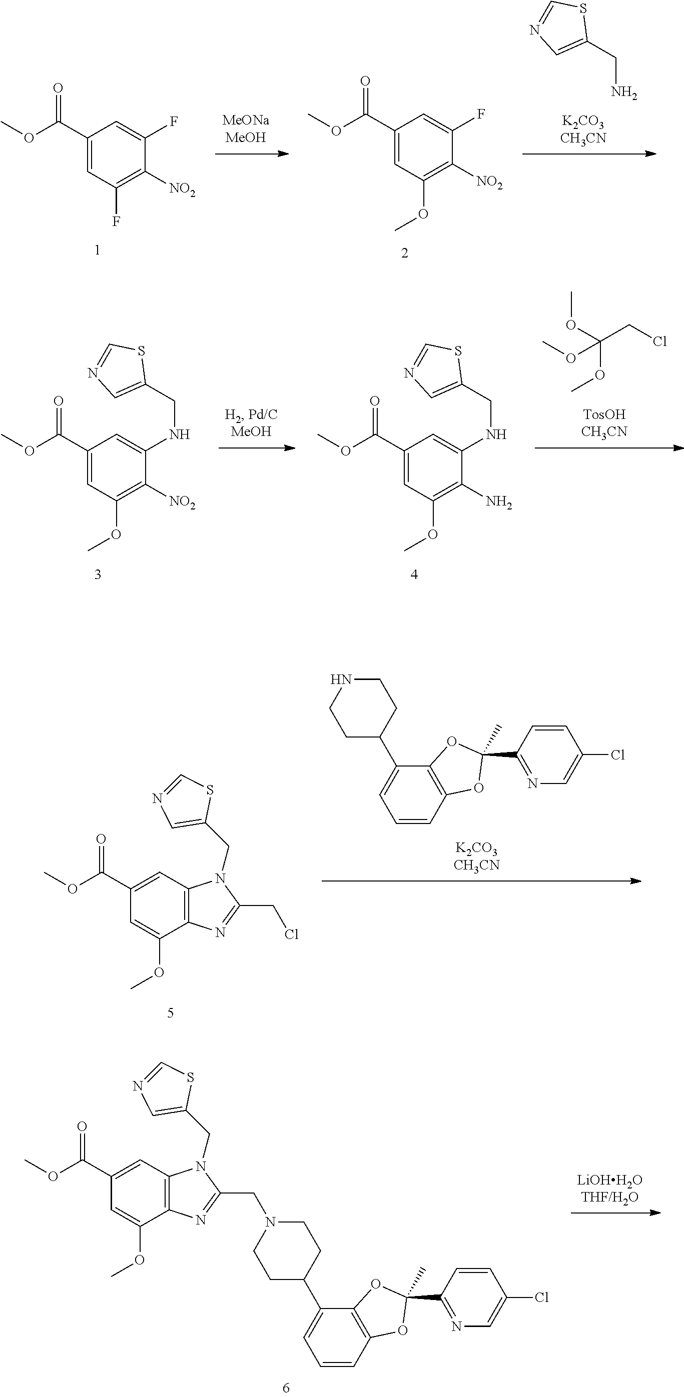

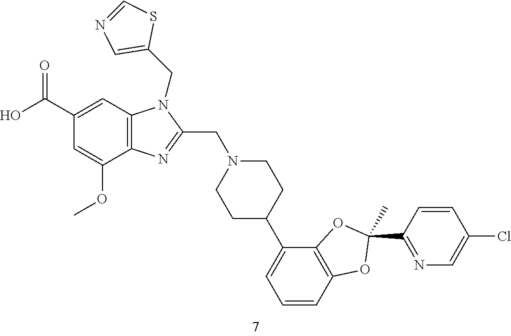 [SMILES: COc1cc(C(=O)O)cc2c1nc(CN1CCC(c3cccc4c3O[C@@](C)(c3ccc(Cl)cn3)O4)CC1)n2Cc1cncs1]

### Preparation of Intermediate Methyl 3-fluoro-5-methoxy-4-nitrobenzoate (2)

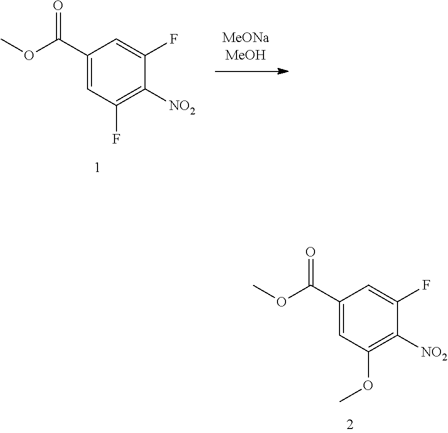 [SMILES: COC(=O)c1cc(F)c([N+](=O)[O-])c(F)c1 ; COC(=O)c1cc(F)c([N+](=O)[O-])c(OC)c1]

Two reactions were performed in parallel.

To a solution of methyl methyl 3,5-difluoro-4-nitrobenzoate 1 (12 g, 55.3 mmol, 1 eq) in MeOH (120 mL) was added MeONa (3.58 g, 66.3 mmol, 1.2 eq) in portions at 0° C. The mixture was stirred at 25° C. for 12 h. TLC indicated starting material was consumed completely and one new spot formed. The reaction was clean according to TLC (Rf=0.51). 2 reactions were combined for workup. The reaction mixture was concentrated under reduced pressure to remove solvent to give a residue. The residue was purified by column chromatography (SiO₂, Petroleum ether/Dichloromethane=2/1 to 0/1) to provide product (21 g, 91.64 mmol, 83% yield) as a light yellow solid.

¹H NMR (400 MHz, DMSO-d6) δ=7.69-7.60 (m, 2H), 4.03 (s, 3H), 3.92 (s, 3H)

### Preparation of Intermediate Methyl 3-methoxy-4-nitro-5-((thiazol-5-ylmethyl)amino)benzoate (3)

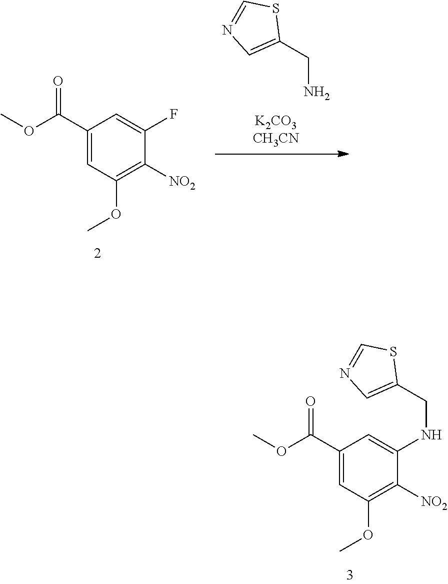 [SMILES: COC(=O)c1cc(F)c([N+](=O)[O-])c(OC)c1 ; NCc1cncs1 ; COC(=O)c1cc(NCc2cncs2)c([N+](=O)[O-])c(OC)c1]

To a solution of methyl 3-fluoro-5-methoxy-4-nitrobenzoate 2 (1.3 g, 5.67 mmol, 1 eq) in ACN (15 mL) was added K₂CO₃ (3.92 g, 28.4 mmol, 5 eq), thiazol-5-ylmethanamine (1.59 g, 8.51 mmol, 1.5 eq, 2HCl). The mixture was stirred at 60° C. for 12 h. TLC indicated ˜50% of starting material remained and one major new spot with larger polarity was detected. The reaction mixture was diluted with H₂O 20 mL and extracted with EtOAc (30 mL*3). The combined organic layers were dried over Na₂SO₄, filtered, and concentrated under reduced pressure to give a residue. The residue was purified by column chromatography (SiO₂, Petroleum ether/Ethyl acetate=10/1 to 0/1) to provide product 0.7 g, 2.17 mmol, 38% yield) as a yellow solid.

LCMS: RT=1.636 min, MS cal.: 323.3, [M+H]⁺=324.0

¹H NMR (400 MHz, CHLOROFORM-d) δ=8.77 (s, 1H), 7.86 (s, 1H), 7.15 (d, J=1.2 Hz, 1H), 7.03 (d, J=1.2 Hz, 1H), 6.09 (br s, 1H), 4.71 (d, J=4.0 Hz, 2H), 3.94 (s, 3H), 3.93 (s, 3H).

### Preparation of Intermediate Methyl 4-amino-3-methoxy-5-((thiazol-5-ylmethyl)amino)benzoate (4)

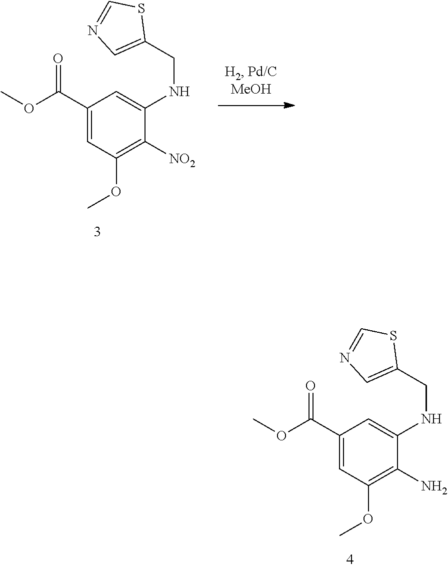 [SMILES: COC(=O)c1cc(NCc2cncs2)c([N+](=O)[O-])c(OC)c1 ; COC(=O)c1cc(NCc2cncs2)c(N)c(OC)c1]

THF (10 mL) and MeOH (3 mL) was charged to the round bottom flask, then methyl 3-methoxy-4-nitro-5-((thiazol-5-ylmethyl)amino)benzoate 3 (0.7 g, 2.17 mmol, 1 eq) was added to the mixture at 20° C. At 20° C., Pd/C (0.3 g, 10%) was added under N₂ atmosphere. The suspension was degassed and purged with H₂ for 3 times. The mixture was stirred under H₂ (15 psi) at 20° C. for 2 h. The reaction was monitored by TLC. The mixture was filtered through a Celite pad, and the filtrate was concentrated to provide product (450 mg, 1.53 mmol, 71% yield) as a white solid.

LCMS: RT=0.919 min, MS cal.: 293.3, [M+H]⁺=294.1

¹H NMR (400 MHz, CHLOROFORM-d) δ=8.79 (s, 1H), 7.84 (s, 1H), 7.22 (s, 2H), 7.21 (s, 2H), 4.60 (br s, 2H), 3.91 (s, 3H), 3.87 (s, 3H).

### Preparation of Intermediate Methyl 2-(chloromethyl)-4-methoxy-1-(thiazol-5-ylmethyl)-1H-benzo[d]imidazole-6-carboxylate (5)

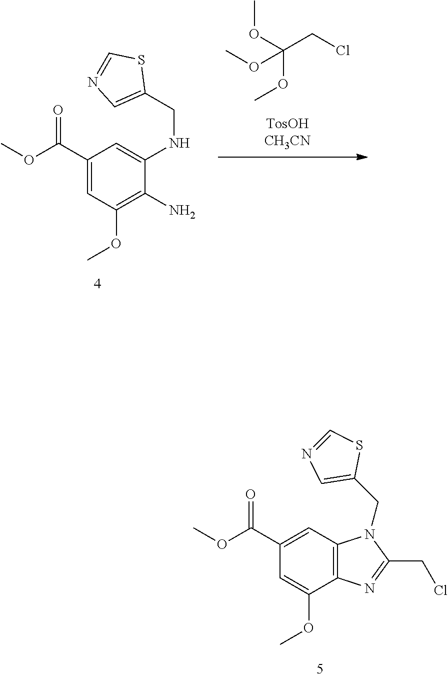

To a solution of methyl 4-amino-3-methoxy-5-((thiazol-5-ylmethyl)amino)benzoate 4 (120 mg, 409 μmol, 1 eq) in ACN (2 mL) was added p-TSA (7.04 mg, 40.9 μmol, 0.1 eq) and 2-chloro-1,1,1-trimethoxyethane (82.2 mg, 532 μmol, 71.7 μL, 1.3 eq). The mixture was stirred at 60° C. for 2 h. LC-MS showed starting material was consumed completely and desired mass was detected. The reaction mixture was concentrated under reduced pressure to remove solvent. The residue was purified by column chromatography (SiO₂, Petroleum ether/Ethyl acetate=10/1 to 0/1) to provide product (140 mg, 398 μmol, 97% yield) as a white solid.

LCMS: RT=0.934 min, MS cal.: 351.8, [M+H]⁺=352.0/354.0

### Preparation of Intermediate Methyl (S)-2-((4-(2-(5-chloropyridin-2-yl)-2-methylbenzo[d][1,3]dioxol-4-yl)piperidin-1-yl)methyl)-4-methoxy-1-(thiazol-5-ylmethyl)-1H-benzo[d]imidazole-6-carboxylate (6)

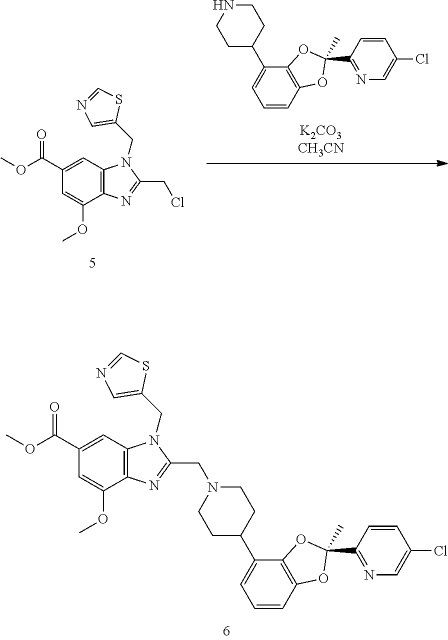 [SMILES: COC(=O)c1cc(OC)c2nc(CCl)n(Cc3cncs3)c2c1 ; C[C@]1(c2ccc(Cl)cn2)Oc2cccc(C3CCNCC3)c2O1 ; COC(=O)c1cc(OC)c2nc(CN3CCC(c4cccc5c4O[C@@](C)(c4ccc(Cl)cn4)O5)CC3)n(Cc3cncs3)c2c1]

To a solution of methyl 2-(chloromethyl)-4-methoxy-1-(thiazol-5-ylmethyl)-1H-benzo[d]imidazole-6-carboxylate 5 (140 mg, 398 μmol, 1 eq) in ACN (3 mL) was added K₂CO₃ (165 mg, 1.19 mmol, 3 eq) and(S)-5-chloro-2-(2-methyl-4-(piperidin-4-yl)benzo[d][1,3]dioxol-2-yl)pyridine (132 mg, 398 μmol, 1 eq). The mixture was stirred at 60° C. for 2 h. LC-MS showed starting material was consumed completely and desired mass was detected. The reaction mixture was poured into H₂O (20 mL) slowly at 0° C. and the solid formed. The mixture was stirred for 15 mins and filtered. The solid was collected and under reduced pressure to give product. The residue was purified by column chromatography (SiO₂, Petroleum ether/Ethyl acetate=10/1 to 0/1) to provide product (210 mg, 325 μmol, 82% yield) as a white solid.

LCMS: RT=2.303 min, MS cal.: 646.1, [M+H]⁺=646.2/647.2

### Preparation of Compound 32 (7)

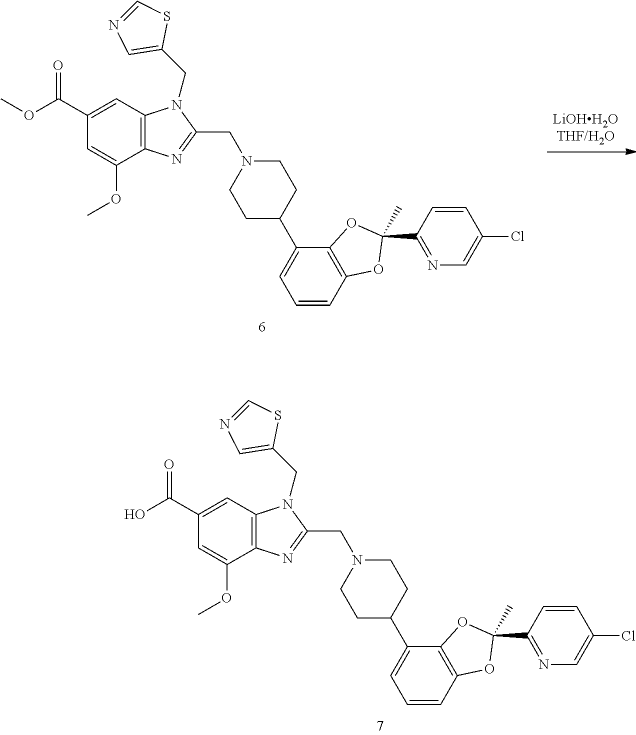 [SMILES: COC(=O)c1cc(OC)c2nc(CN3CCC(c4cccc5c4O[C@@](C)(c4ccc(Cl)cn4)O5)CC3)n(Cc3cncs3)c2c1 ; COc1cc(C(=O)O)cc2c1nc(CN1CCC(c3cccc4c3O[C@@](C)(c3ccc(Cl)cn3)O4)CC1)n2Cc1cncs1]

To a solution of methyl (S)-2-((4-(2-(5-chloropyridin-2-yl)-2-methylbenzo[d][1,3]dioxol-4-yl)piperidin-1-yl)methyl)-4-methoxy-1-(thiazol-5-ylmethyl)-1H-benzo[d]imidazole-6-carboxylate 6 (100 mg, 155 μmol, 1 eq) in THF (0.7 mL) and H₂O (0.3 mL) was added LiOH·H₂O (9.74 mg, 232 μmol, 1.5 eq). The mixture was stirred at 20° C. for 12 h. LCMS showed the starting material has been consumed and a new main peak with desired mass was detected. The reaction mixture was dried in a stream of N₂ to remove most of the THF, and the rest residue was purified by prep-HPLC under neutral condition (column: Waters Xbridge Prep OBD C₁₈ 150*40 mm*10 um; mobile phase: [H₂O (10 mM NH₄HCO₃)-ACN]; gradient: 20%-60% B over 8.0 min) to provide (29.6 mg, 46.8 μmol, 30% yield) as a white solid.

LCMS: RT=2.631 min, MS cal.: 632.1, [M+H]⁺=632.2/634.2

HPLC: RT=10.395 min, purity: 100.00%

1H NMR (400 MHz, DMSO-d₆) δ=8.96 (s, 1H), 8.72 (d, J=2.4 Hz, 1H), 8.04-7.99 (m, 2H), 7.81 (s, 1H), 7.60 (d, J=8.4 Hz, 1H), 7.28 (s, 1H), 6.83-6.76 (m, 2H), 6.75-6.68 (m, 1H), 5.91 (s, 2H), 3.96 (s, 3H), 3.84 (s, 2H), 2.96-2.94 (m, 2H), 2.66-2.59 (m, 1H), 2.23-2.14 (m, 2H), 2.01 (s, 3H), 1.75-1.62 (m, 4H).

### Example A28. —Preparation of Compound 87. (S)-2-((4-(2-(5-Chloropyridin-2-yl)-2-methylbenzo[d][1,3]dioxol-4-yl)piperidin-1-yl)methyl)-4-(1,1-difluoroethyl)-1-methyl-1H-benzo[d]imidazole-6-carboxylic acid (Compound 87) (7)

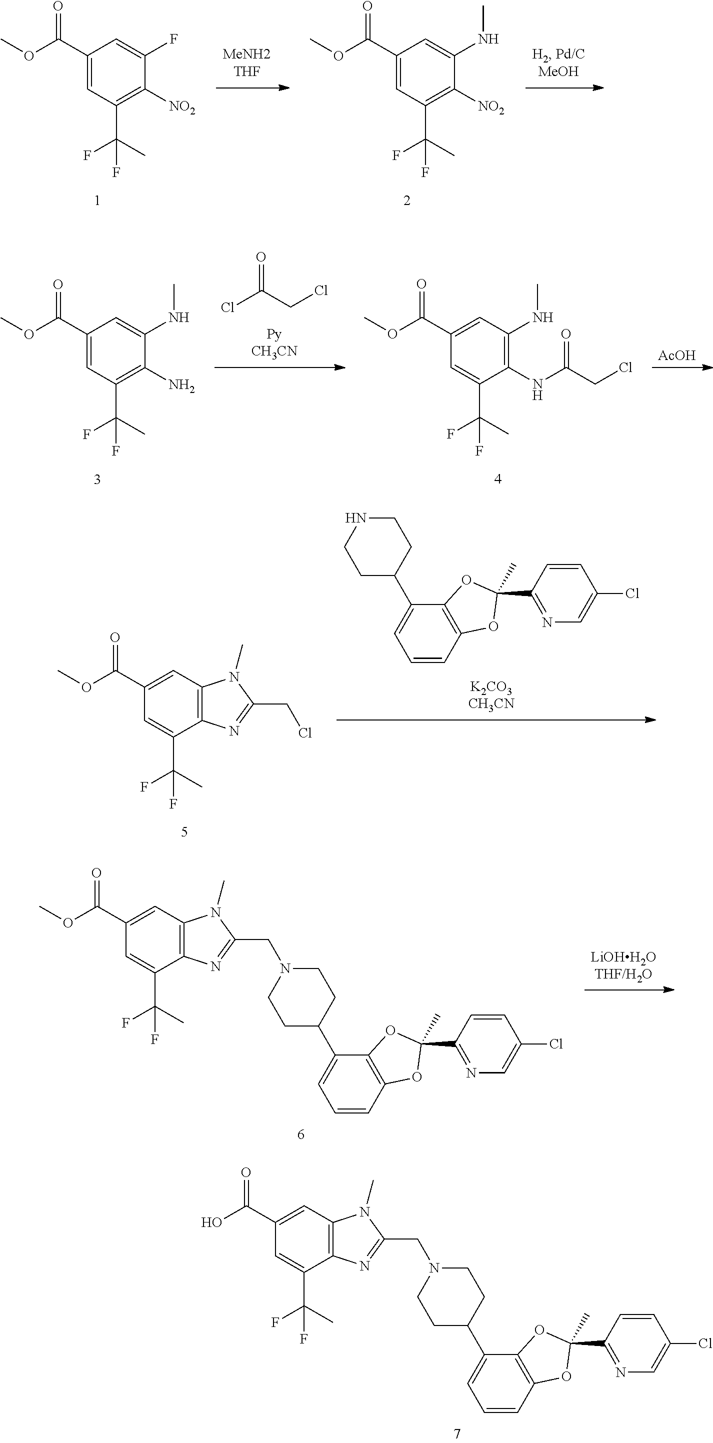

### Preparation of Intermediate Methyl 3-(1,1-difluoroethyl)-5-(methylamino)-4-nitrobenzoate (2)

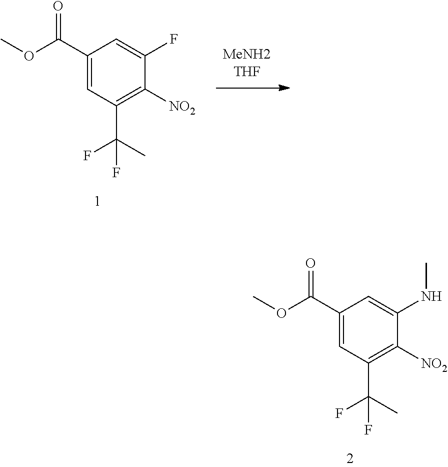 [SMILES: COC(=O)c1cc(F)c([N+](=O)[O-])c(C(C)(F)F)c1 ; CNc1cc(C(=O)OC)cc(C(C)(F)F)c1[N+](=O)[O-]]

To a solution of methyl 3-(1,1-difluoroethyl)-5-fluoro-4-nitrobenzoate 1 (1 g, 3.80 mmol, 1 eq) in THF (10 mL) was added dropwise CH₂NH₂·THF (2 M, 3.80 mL, 2 eq) at 20° C. The mixture was stirred at 20° C. for 12 h. TLC indicated starting material was consumed completely. The reaction mixture was added FA (1 M) to adjust pH=4, and extracted with EtOAc 30 mL (10 mL*3). The combined organic layers were washed with brine 30 mL, dried over Na₂SO₄, filtered, and concentrated under reduced pressure to give a residue. The residue was purified by column chromatography (SiO₂, Petroleum ether/Ethyl acetate=100/1 to 20/1). The residue was repurified by prep-HPLC (neutral condition; column: Phenomenex Gemini-NX 80*40 mm*3 um; mobile phase: [H₂O (10 mM NH₄HCO₃)-ACN]; gradient: 25%-50% B over 8.0 min) to provide product (300 mg, 1.09 mmol, 29% yield) as a red solid.

LCMS: RT=1.223 min, MS cal.: 274.2, [M+H]⁺=275.0

¹H NMR (400 MHz, CHLOROFORM-d) δ=7.53 (s, 1H), 7.50 (d, J=1.6 Hz, 1H), 5.06 (br s, 1H), 3.97 (s, 3H), 2.97 (s, 3H), 2.10 (t, J=18.4 Hz, 3H)

### Preparation of Intermediate Methyl 4-amino-3-(1,1-difluoroethyl)-5-(methylamino)benzoate (3)

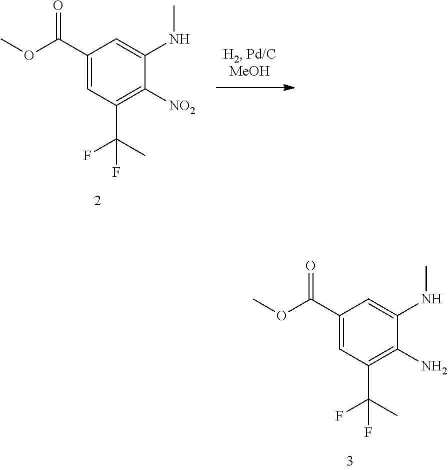

To a solution of methyl 3-(1,1-difluoroethyl)-5-(methylamino)-4-nitrobenzoate 2 (200 mg, 729 μmol, 1 eq) in MeOH (1 mL) was added Pd/C (50 mg, 10%) in MeOH (1 mL) under N₂ atmosphere. The suspension was degassed and purged with H₂ for 3 times. The mixture was stirred under H₂ (15 Psi) at 20° C. for 1 hour. TLC indicated starting material was consumed completely. The reaction mixture was filtered by kieselguhr and the filtrate was concentrated under reduced pressure to give a residue. The residue was purified by column chromatography (SiO₂, Petroleum ether/Ethyl acetate=100/1 to 10/1) to provide product (110 mg, 450 μmol, 62% yield) as a white solid.

LCMS: RT=0.416 min, MS cal.: 244.2, [M+H]⁺=245.0

### Preparation of Intermediate Methyl 4-(2-chloroacetamido)-3-(1,1-difluoroethyl)-5-(methylamino)benzoate (4)

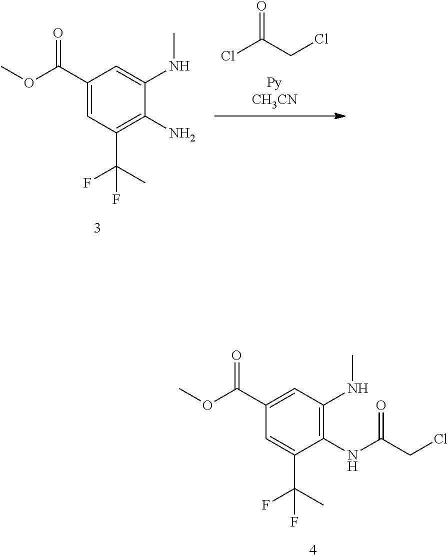

To a solution of methyl methyl 4-amino-3-(1,1-difluoroethyl)-5-(methylamino)benzoate 3 (110 mg, 450 μmol, 1 eq) in ACN (1 mL) was added pyridine (356 mg, 4.50 mmol, 363 μL, 10 eq) at 25° C. Then the mixture was cooled to 0° C. and added 2-chloroacetyl chloride (61.0 mg, 540 μmol, 43.1 μL, 1.2 eq). The mixture was stirred at 25° C. for 2 h. TLC (Petroleum ether/Ethyl acetate=2:1, R_{f}=0.33) indicated starting material was consumed completely and one new spot formed. The reaction was clean according to TLC. The reaction mixture was diluted with H₂O 10 mL and extracted with DCM 30 mL (10 mL*3). The organic phase was combined, washed with brine 10 mL, dried over Na₂SO₄, filtered, and concentrated under reduced pressure to provide product (140 mg, 437 μmol, 97% yield) as a yellow solid.

LCMS: RT=1.0.48 min, MS cal.: 320.1, 322.1, [M+H]⁺=321.0, 323.0

### Preparation of Intermediate Methyl 2-(chloromethyl)-4-(1,1-difluoroethyl)-1-methyl-1H-benzo[d]imidazole-6-carboxylate (5)

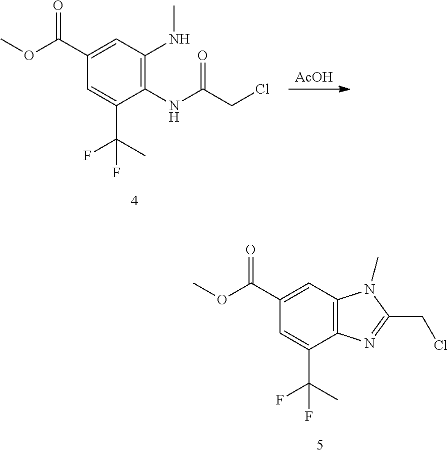 [SMILES: "AcOH" ; CNc1cc(C(=O)OC)cc(C(C)(F)F)c1NC(=O)CCl ; COC(=O)c1cc(C(C)(F)F)c2nc(CCl)n(C)c2c1]

A mixture of methyl 4-(2-chloroacetamido)-3-(1,1-difluoroethyl)-5-(methylamino)benzoate 4 (140 mg, 436.52 μmol, 1 eq) in AcOH (1 mL) was degassed and purged with N₂ for 3 times, and then the mixture was stirred at 60° C. for 1 h under N₂ atmosphere. TLC (Petroleum ether/Ethyl acetate=2/1, R_{f}=0.60) indicated starting material was consumed completely and one new spot formed. The reaction was clean according to TLC. The reaction mixture was concentrated under reduced pressure to provide product (130 mg, 429 μmol, 98% yield) as a yellow oil.

LCMS: RT=1.126 min, MS cal.: 302.1, 304.1, [M+H]⁺=303.0, 305.0

¹H NMR (400 MHz, CHLOROFORM-d) δ=8.21 (s, 1H), 8.20 (s, 1H), 4.92 (s, 2H), 3.99 (s, 3H), 3.97 (s, 3H), 2.24 (t, J=18.4 Hz, 3H).

### Preparation of Intermediate Methyl (S)-2-((4-(2-(5-Chloropyridin-2-yl)-2-methylbenzo[d][1,3]dioxol-4-yl)piperidin-1-yl)methyl)-4-(1,1-difluoroethyl)-1-methyl-1H-benzo[d]imidazole-6-carboxylate (6)

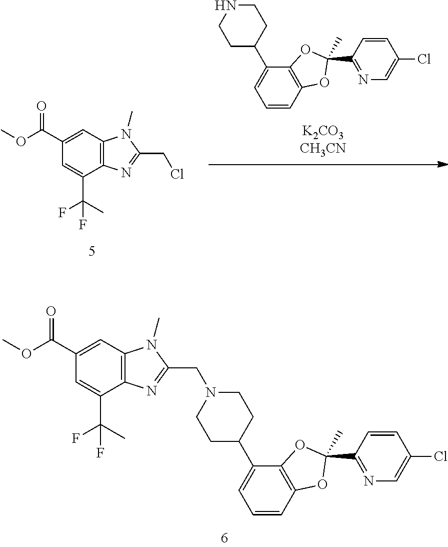 [SMILES: COC(=O)c1cc(C(C)(F)F)c2nc(CCl)n(C)c2c1 ; C[C@]1(c2ccc(Cl)cn2)Oc2cccc(C3CCNCC3)c2O1 ; COC(=O)c1cc(C(C)(F)F)c2nc(CN3CCC(c4cccc5c4O[C@@](C)(c4ccc(Cl)cn4)O5)CC3)n(C)c2c1]

To a solution of methyl 2-(chloromethyl)-4-(1,1-difluoroethyl)-1-methyl-1H-benzo[d]imidazole-6-carboxylate 5 (130 mg, 429 μmol, 1 eq) in ACN (2 mL) was added K₂CO₃ (178 mg, 1.29 mmol, 3 eq) and(S)-5-chloro-2-(2-methyl-4-(piperidin-4-yl)benzo[d][1,3]dioxol-2-yl)pyridine (156 mg, 472 μmol, 1.1 eq). The mixture was stirred at 60° C. for 12 h. TLC (Petroleum ether/Ethyl acetate=1/1, R_{f}=0.30) indicated starting material was consumed completely and one new spot formed. The reaction was clean according to TLC. The reaction mixture was diluted with H₂O 10 mL and extracted with EtOAc 30 mL (10 mL*3). The organic phase was separated, washed with brine 10 mL, dried over Na₂SO₄, filtered, and concentrated under reduced pressure to give a residue. The residue was purified by column chromatography (SiO₂, Petroleum ether/Ethyl acetate=10/1 to 3/1) to provide product (130 mg, 218 μmol, 51% yield) as a yellow solid.

LCMS: RT=1.637 min, MS cal.: 596.2, 597.2, [M+H]⁺=597.2, 599.2

¹H NMR (400 MHz, CHLOROFORM-d) δ=8.62 (d, J=2.0 Hz, 1H), 8.19 (d, J=8.0 Hz, 2H), 7.68 (dd, J=2.4, 8.4 Hz, 1H), 7.57 (d, J=8.4 Hz, 1H), 6.82-6.77 (m, 1H), 6.74-6.69 (m, 2H), 4.01 (s, 3H), 3.99 (s, 3H) 3.97-3.91 (m, 2H), 2.97-2.95 (m, 2H), 2.78-2.76 (m, 1H), 2.35-2.32 (m, 2H), 2.26 (t, J=18.8 Hz, 4H), 2.05 (s, 3H), 1.94-1.73 (m, 4H).

### Preparation of (S)-2-((4-(2-(5-Chloropyridin-2-yl)-2-methylbenzo[d][1,3]dioxol-4-yl)piperidin-1-yl)methyl)-4-(1,1-difluoroethyl)-1-methyl-1H-benzo[d]imidazole-6-carboxylic acid, Compound 87 (7)

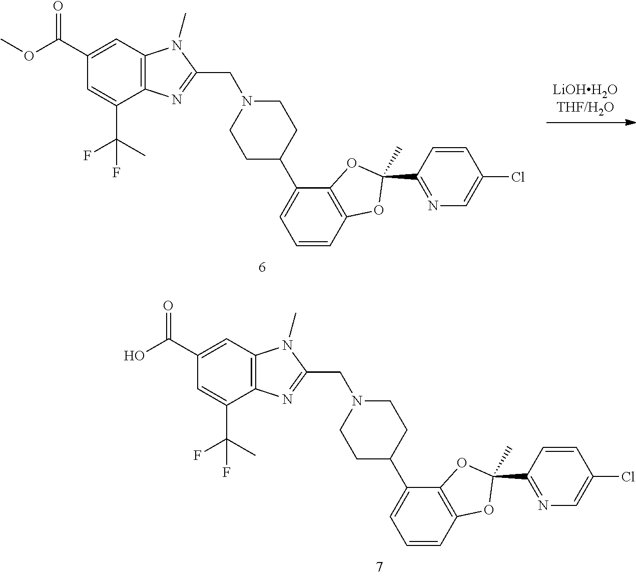 [SMILES: COC(=O)c1cc(C(C)(F)F)c2nc(CN3CCC(c4cccc5c4O[C@@](C)(c4ccc(Cl)cn4)O5)CC3)n(C)c2c1 ; Cn1c(CN2CCC(c3cccc4c3O[C@@](C)(c3ccc(Cl)cn3)O4)CC2)nc2c(C(C)(F)F)cc(C(=O)O)cc21]

To a solution of methyl (S)-2-((4-(2-(5-chloropyridin-2-yl)-2-methylbenzo[d][1,3]dioxol-4-yl)piperidin-1-yl)methyl)-4-(1,1-difluoroethyl)-1-methyl-1H-benzo[d]imidazole-6-carboxylate 6 (120 mg, 201 μmol, 1 eq) in THF (0.7 mL) was added LiOH·H₂O (12.7 mg, 301 μmol, 1.5 eq) in H₂O (0.3 mL). The mixture was stirred at 25° C. for 12 h. LC-MS showed starting material was consumed completely and desired mass was detected. The mixture was purified directly. The residue was purified by prep-HPLC (FA condition, column: Phenomenex Luna C18 100*30 mm*3 um; mobile phase: [H₂O (0.2% FA)-ACN]; gradient: 15%-55% B over 8.0 min) to provide product (29.5 mg, 50.5 μmol, 25% yield) as a white solid.

LCMS: RT=1.495 min, MS cal.: 583.03, [M+H]⁺=583.1

LCMS: RT=2.438 min, MS cal.: 583.03, [M+H]⁺=583.2

HPLC: RT=11.857 min, purity: 97.27%

¹H NMR (400 MHz, METHANOL-d4) δ=8.59 (d, J=2.0 Hz, 1H), 8.30 (s, 1H), 8.11 (s, 1H), 7.86 (dd, J=2.4, 8.4 Hz, 1H), 7.64 (d, J=8.4 Hz, 1H), 6.81-6.68 (m, 3H), 4.06 (s, 2H), 4.02 (s, 3H), 3.16-3.14 (m, 2H), 2.83-2.74 (m, 1H), 2.47-2.44 (m, 2H), 2.18 (t, J=18.4 Hz, 3H), 2.01 (s, 3H), 1.98-1.81 (m, 4H).

### Example A29. —Alternate preparation of Compound 84. (S)-2-((4-(2-(5-Chloropyridin-2-yl)-2-methylbenzo[d][1,3]dioxol-4-yl)piperidin-1-yl)methyl)-4-isopropoxy-1-methyl-1H-benzo[d]imidazole-6-carboxylic acid (Compound 84) (7)

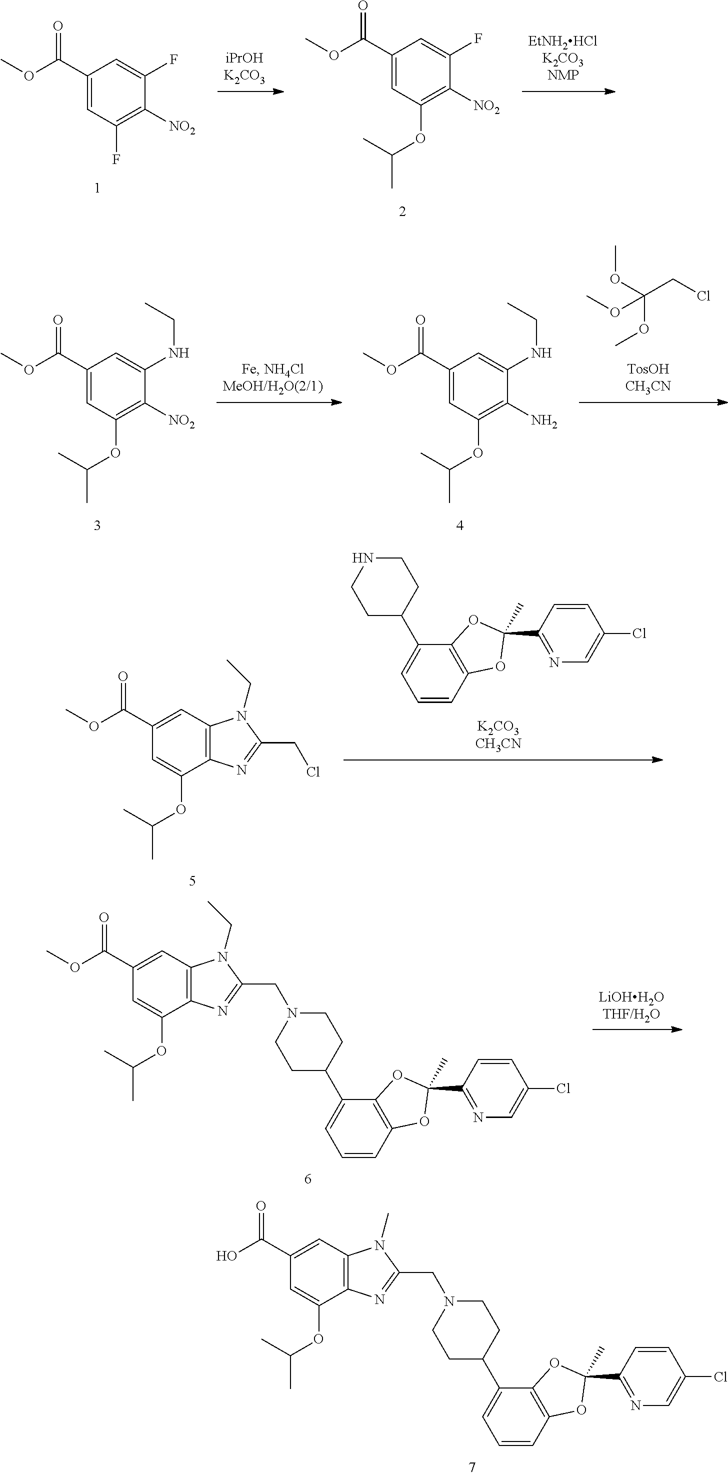

### Preparation of Intermediate Methyl 3-fluoro-5-isopropoxy-4-nitrobenzoate (2)

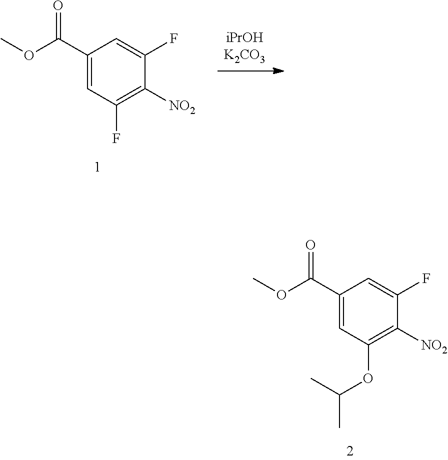 [SMILES: COC(=O)c1cc(F)c([N+](=O)[O-])c(F)c1 ; COC(=O)c1cc(F)c([N+](=O)[O-])c(OC(C)C)c1]

A mixture of methyl 3,5-difluoro-4-nitrobenzoate 1 (5 g, 23.0 mmol, 1 eq) and K₂CO₃ (9.55 g, 69.1 mmol, 3 eq) in i-PrOH (50 mL) was degassed and purged with N₂ for 3 times, then the mixture was stirred at 90° C. for 12 h under N₂ atmosphere. LCMS showed the starting material was consumed completely. The reaction mixture was quenched by H₂O 30 mL at 20° C., extracted with ethyl acetate 150 mL (50 mL*3). The combined organic layers were washed with brine 40 mL (20 mL*2), dried over Na₂SO₄, filtered, and concentrated under reduced pressure to give a residue. The residue was purified by column chromatography (SiO₂, Petroleum ether/Ethyl acetate=100/1 to 90/1) to provide product (1.7 g, 5.96 mmol, 26% yield) as a colorless oil.

¹H NMR (400 MHz, CHLOROFORM-d) δ=7.51 (d, J=1.2 Hz, 1H), 7.44 (dd, J=1.2, 9.3 Hz, 1H), 5.32-5.20 (m, 1H), 4.79-4.73 (m, 1H), 1.40 (d, J=6.0 Hz, 6H).

### Preparation of Intermediate Methyl 3-(ethylamino)-5-isopropoxy-4-nitrobenzoate (3)

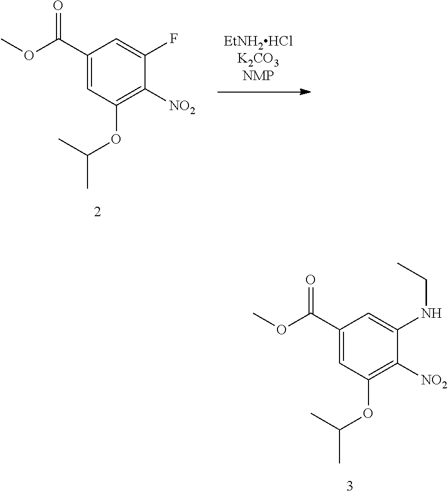 [SMILES: COC(=O)c1cc(F)c([N+](=O)[O-])c(OC(C)C)c1 ; CCNc1cc(C(=O)OC)cc(OC(C)C)c1[N+](=O)[O-]]

A mixture of methyl 3-fluoro-5-isopropoxy-4-nitrobenzoate 2 (800 mg, 2.80 mmol, 1 eq), ethylamine hydrochloride (662 mg, 5.61 mmol, 2 eq, HCl) and K₂CO₃ (1.94 g, 14.0 mmol, 5 eq) in NMP (8 mL) was degassed and purged with N₂ for 3 times, and then the mixture was stirred at 25° C. for 48 h under N₂ atmosphere. HPLC showed the starting material was consumed completely. The reaction mixture was quenched by H₂O 5 mL at 20° C., extracted with ethyl acetate 15 mL (5 mL*3). The combined organic layers were washed with brine 10 mL (5 mL*2), dried over Na₂SO₄, filtered, and concentrated under reduced pressure to give a residue. The residue was purified by column chromatography (SiO₂, Petroleum ether/Ethyl acetate=100/1 to 50/1) to provide product (700 mg, 2.26 mmol, 80% yield) as a yellow oil.

HPLC: RT=4.014 min.

¹H NMR (400 MHz, CHLOROFORM-d) δ=7.01 (d, J=1.4 Hz, 1H), 6.92 (d, J=1.4 Hz, 1H), 5.27-5.21 (m, 1H), 4.70-4.67 (m, 1H), 3.27 (q, J=7.2 Hz, 2H), 1.38 (d, J=6.0 Hz, 12H), 1.30 (t, J=7.2 Hz, 3H).

### Preparation of Intermediate Methyl 4-amino-3-(ethylamino)-5-isopropoxybenzoate (4)

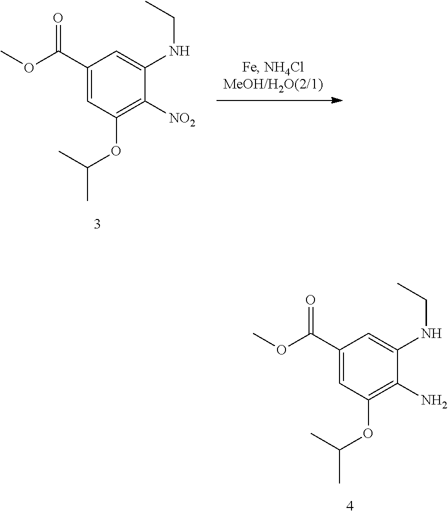 [SMILES: CCNc1cc(C(=O)OC)cc(OC(C)C)c1[N+](=O)[O-] ; CCNc1cc(C(=O)OC)cc(OC(C)C)c1N]

MeOH (4 mL) was charged to the three-necked round bottom flask, then methyl 3-(ethylamino)-5-isopropoxy-4-nitrobenzoate 3 (350 mg, 1.13 mmol, 1 eq) and NH₄Cl (301.63 mg, 5.64 mmol, 5 eq) in H₂O (2 mL) was added to the mixture at 20° C. Fe (315 mg, 5.64 mmol, 5 eq) was added in portions to the reaction mixture at 20° C. After the addition, the mixture was stirred at 70° C. for 1 h. LCMS showed the starting material was consumed completely. The reaction mixture was filtered and the filtrate was added to H₂O 5 mL at 20°. The mixture was extracted with ethyl acetate (5 mL*3). The combined organic phase was washed with brine (5 mL*2), dried with Na₂SO₄, filtered. The organic phase was concentrated under reduced pressure at 35° C. to give a residue. The residue was purified by column chromatography (SiO₂, Petroleum ether/Ethyl acetate=100/1 to 50/1) to provide product (280 mg, 999 μmol, 89% yield) as a white solid.

LCMS: RT=0.421 min, MS cal.: 280.2, [M+H]⁺=281.1

¹H NMR (400 MHz, CHLOROFORM-d) δ=7.18 (d, J=1.2 Hz, 1H), 7.11 (d, J=1.2 Hz, 1H), 5.27-5.16 (m, 1H), 4.67-4.55 (m, 1H), 3.75 (br s, 2H), 3.22 (q, J=7.2 Hz, 2H), 1.36 (d, J=6.0 Hz, 12H), 1.30 (t, J=7.2 Hz, 3H).

### Preparation of Intermediate Methyl 2-(chloromethyl)-1-ethyl-4-isopropoxy-1H-benzo[d]imidazole-6-carboxylate (5)

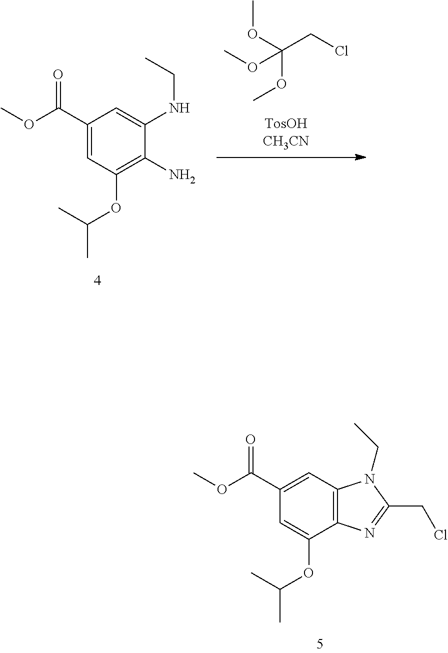

A mixture of methyl 4-amino-3-(ethylamino)-5-isopropoxybenzoate 4 (220 mg, 785 μmol, 1 eq), 2-chloro-1,1,1-trimethoxyethane (243 mg, 1.57 mmol, 212 μL, 2 eq), p-TSA (27.0 mg, 157 μmol, 0.2 eq) in CH₃CN (2.2 mL) was degassed and purged with N₂ for 3 times, and then the mixture was stirred at 60° C. for 2 h under N₂ atmosphere. The reaction mixture was concentrated under reduced pressure to provide product (300 mg, crude) as a colorless oil.

### Preparation of Intermediate (S)-2-((4-(2-(5-chloropyridin-2-yl)-2-methylbenzo[d][1,3] dioxol-4-yl)piperidin-1-yl)methyl)-4-isopropoxy-1-methyl-1H-benzo[d]imidazole-6-carboxylic acid (6)

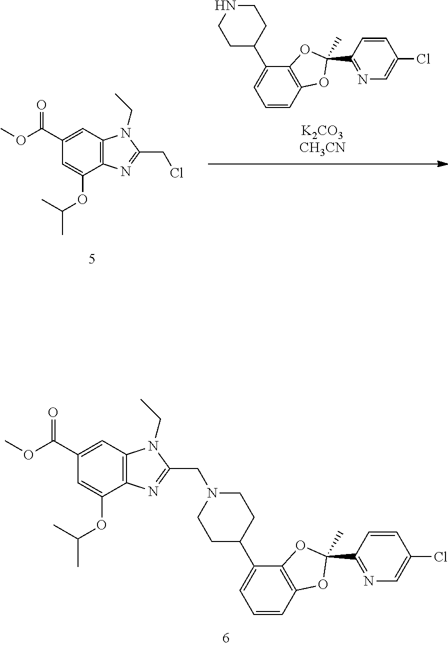 [SMILES: CCn1c(CCl)nc2c(OC(C)C)cc(C(=O)OC)cc21 ; C[C@]1(c2ccc(Cl)cn2)Oc2cccc(C3CCNCC3)c2O1 ; CCn1c(CN2CCC(c3cccc4c3O[C@@](C)(c3ccc(Cl)cn3)O4)CC2)nc2c(OC(C)C)cc(C(=O)OC)cc21]

A mixture of methyl 2-(chloromethyl)-1-ethyl-4-isopropoxy-1H-benzo[d]imidazole-6-carboxylate 5 (100 mg, 295.13 μmol, 1 eq), (S)-5-chloro-2-(2-methyl-4-(piperidin-4-yl)benzo[d][1,3]dioxol-2-yl)pyridine (117 mg, 354 μmol, 1.2 eq) and K₂CO₃ (204 mg, 1.48 mmol, 5 eq) in CH₃CN (3 mL) was degassed and purged with N₂ for 3 times, then the mixture was stirred at 60° C. for 4 h under N₂ atmosphere. The reaction mixture was quenched by H₂O 5 mL at 20° C., extracted with ethyl acetate 15 mL (5 mL*3). The combined organic layers were washed with brine 10 mL (5 mL*2), dried over Na₂SO₄, filtered, and concentrated under reduced pressure to give a residue. The residue was purified by column chromatography (SiO₂, Petroleum ether/Ethyl acetate=75/1 to 50/1) to provide product (120 mg, 190 μmol, 64.22% yield) as a white solid.

¹H NMR (400 MHz, CHLOROFORM-d) δ=8.62 (d, J=2.0 Hz, 1H), 7.72-7.65 (m, 2H), 7.59-7.57 (m, 1H), 7.40 (s, 1H), 6.82-6.75 (m, 1H), 6.73-6.67 (m, 2H), 5.34-5.25 (m, 1H), 5.02-4.98 (m, 1H), 4.43-4.40 (m, 2H), 3.89 (s, 2H), 2.98-2.96 (m, 2H), 2.83-2.67 (m, 1H), 2.39-2.22 (m, 2H), 2.05 (s, 3H), 1.94-1.70 (m, 4H), 1.53-1.50 (m, 3H), 1.49 (d, J=6.0 Hz, 6H), 1.41 (d, J=6.4 Hz, 6H).

### Preparation of (S)-2-((4-(2-(5-chloropyridin-2-yl)-2-methylbenzo[d][1,3]dioxol-4-yl)piperidin-1-yl)methyl)-4-isopropoxy-1-methyl-1H-benzo[d]imidazole-6-carboxylic acid, Compound 84 (7)

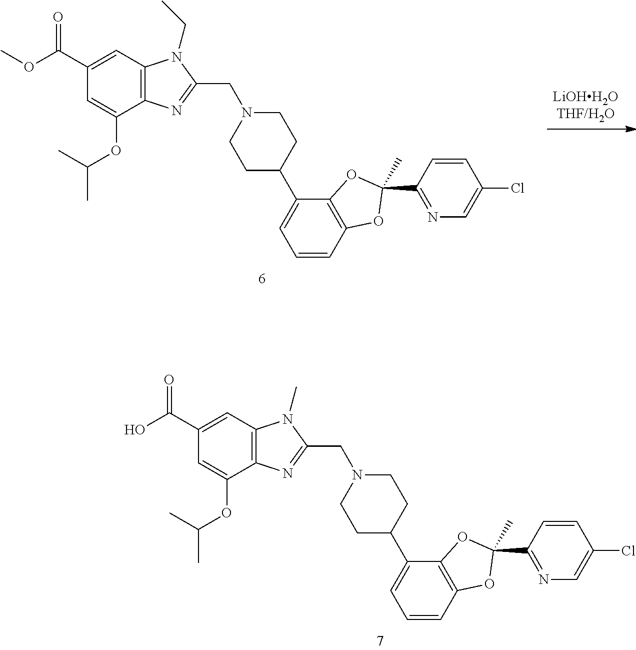 [SMILES: CCn1c(CN2CCC(c3cccc4c3O[C@@](C)(c3ccc(Cl)cn3)O4)CC2)nc2c(OC(C)C)cc(C(=O)OC)cc21 ; CC(C)Oc1cc(C(=O)O)cc2c1nc(CN1CCC(c3cccc4c3O[C@@](C)(c3ccc(Cl)cn3)O4)CC1)n2C]

A mixture of methyl (S)-2-((4-(2-(5-chloropyridin-2-yl)-2-methylbenzo[d][1,3]dioxol-4-yl)piperidin-1-yl)methyl)-1-ethyl-4-isopropoxy-1H-benzo[d]imidazole-6-carboxylate 6 (120 mg, 190 μmol, 1 eq), LiOH·H₂O (11.9 mg, 284 μmol, 1.5 eq) in THF (0.4 mL), H₂O (0.4 mL) and MeOH (0.4 mL) was degassed and purged with N₂ for 3 times, and then the mixture was stirred at 70° C. for 12 h under N₂ atmosphere. LCMS showed the starting material was consumed completely. The reaction was purified by prep-HPLC (column: Waters Xbridge Prep OBD C18 150*40 mm*10 um; mobile phase: [H₂O (10 mM NH₄HCO₃)—CH₃CN]; gradient: 15%-65% B over 8.0 min) to provide product (26.7 mg, 45.1 μmol, 24% yield, 100% purity) as a white solid.

LCMS: RT=2.718 min, MS cal.: 590.2, [M+H]⁺=591.3

HPLC: RT=11.698 min, purity: 100%

¹H NMR (400 MHz, DMSO-d6) δ=8.72 (dd, J=0.8, 2.4 Hz, 1H), 8.00 (dd, J=2.6, 8.5 Hz, 1H), 7.73 (d, J=1.2 Hz, 1H), 7.63-7.54 (m, 1H), 7.24 (d, J=1.0 Hz, 1H), 6.83-6.65 (m, 3H), 5.05-5.02 (m, 1H), 4.37 (q, J=7.0 Hz, 2H), 3.79 (s, 2H), 2.95-2.92 (m, 2H), 2.72-2.58 (m, 1H), 2.23-2.19 (m, 2H), 1.99 (s, 3H), 1.81-1.61 (m, 4H), 1.39 (t, J=7.2 Hz, 3H), 1.33 (d, J=6.0 Hz, 6H).

### Example A30. —Preparation of Compound 90. (S)-2-((4-(2-(5-chloropyridin-2-yl)-2-methylbenzo[d][1,3]dioxol-4-yl)piperidin-1-yl)methyl)-4-hydroxy-1-(thiazol-5-ylmethyl)-1H-benzo[d]imidazole-6-carboxylic acid (Compound 90) (4)

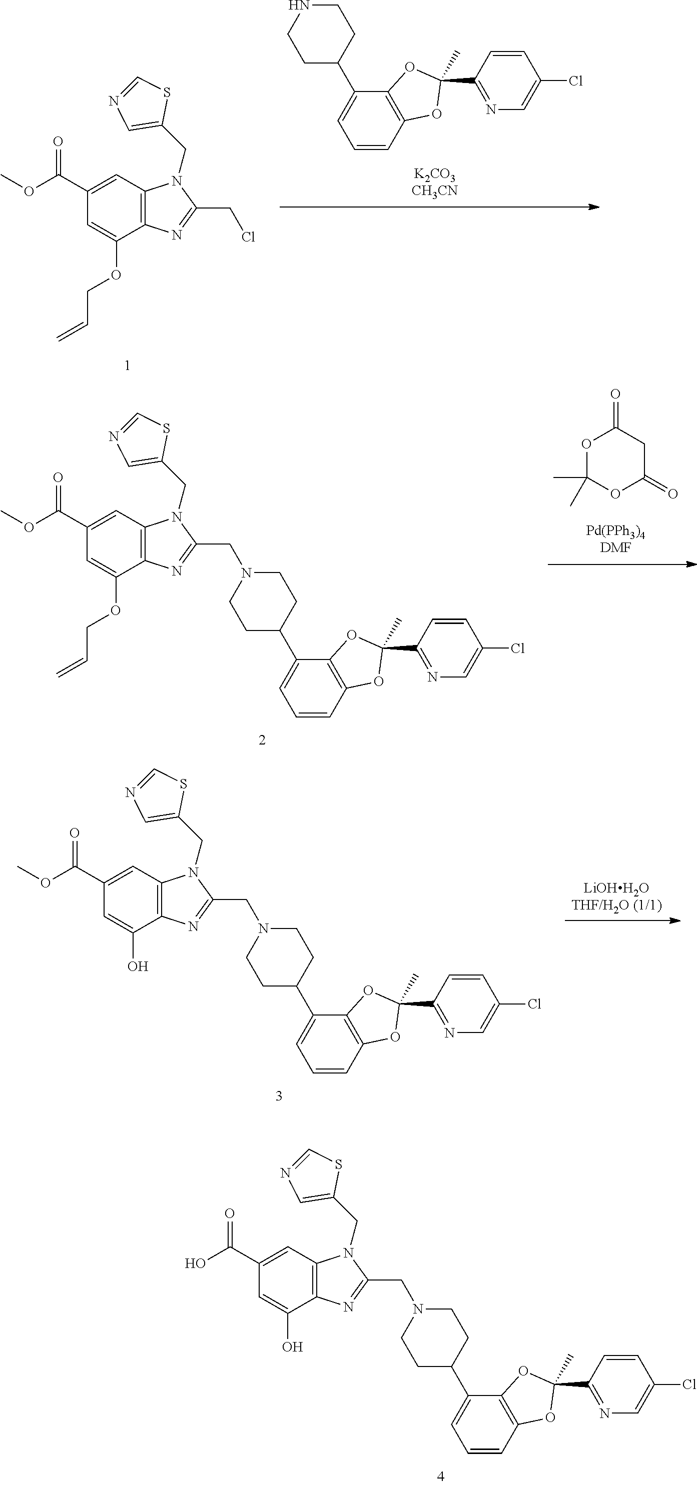

### Preparation of Intermediate Methyl (S)-4-(allyloxy)-2-((4-(2-(5-chloropyridin-2-yl)-2-methylbenzo[d][1,3]dioxol-4-yl)piperidin-1-yl)methyl)-1-(thiazol-5-ylmethyl)-1H-benzo[d]imidazole-6-carboxylate (2)

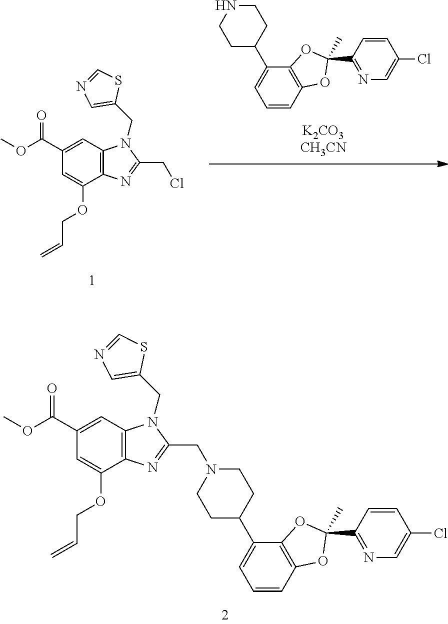 [SMILES: C=CCOc1cc(C(=O)OC)cc2c1nc(CCl)n2Cc1cncs1 ; C[C@]1(c2ccc(Cl)cn2)Oc2cccc(C3CCNCC3)c2O1 ; C=CCOc1cc(C(=O)OC)cc2c1nc(CN1CCC(c3cccc4c3O[C@@](C)(c3ccc(Cl)cn3)O4)CC1)n2Cc1cncs1]

To a solution of methyl 4-(allyloxy)-2-(chloromethyl)-1-(thiazol-5-ylmethyl)-1H-benzo[d]imidazole-6-carboxylate 1 (193 mg, 582 μmol, 1.1 eq) in CH₃CN (2 mL) was added K₂CO₃ (219 mg, 1.59 mmol, 3 eq) and(S)-5-chloro-2-(2-methyl-4-(piperidin-4-yl)benzo[d][1,3]dioxol-2-yl)pyridine (200 mg, 529 μmol, 1 eq). The mixture was stirred at 60° C. for 4 h. The reaction mixture was diluted with H₂O 10 mL and extracted with ethyl acetate (10 mL*3). The combined organic layers were washed with brine 10 mL, dried over Na₂SO₄, filtered, and concentrated under reduced pressure to give a residue. The residue was purified by column chromatography (SiO₂, Petroleum ether/Ethyl acetate=1/0 to 1/1) to provide product (270 mg, 402 μmol, 76% yield) as a yellow solid.

### Preparation of Intermediate Methyl (S)-2-((4-(2-(5-Chloropyridin-2-yl)-2-methylbenzo[d][1,3]dioxol-4-yl)piperidin-1-yl)methyl)-4-hydroxy-1-(thiazol-5-ylmethyl)-1H-benzo[d]imidazole-6-carboxylate (3)

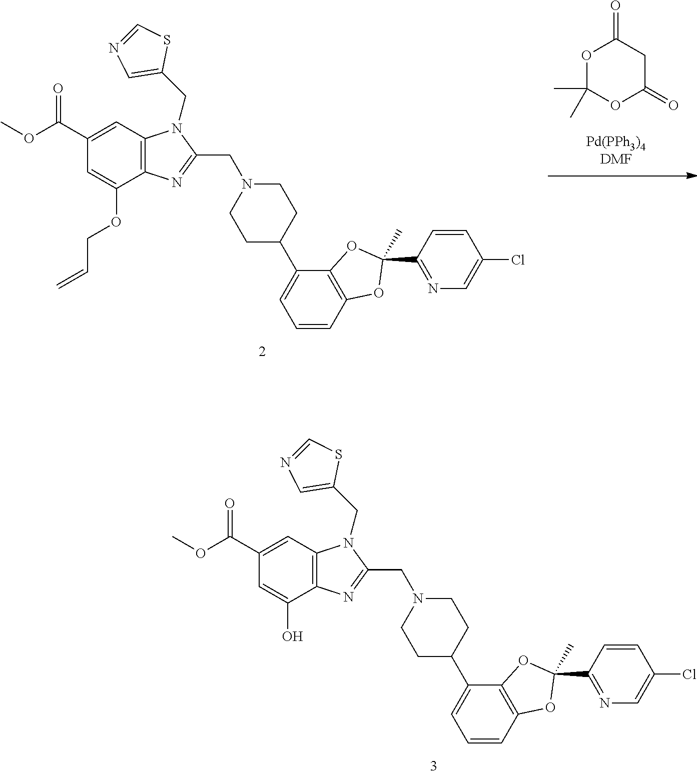

To a solution of methyl (S)-4-(allyloxy)-2-((4-(2-(5-chloropyridin-2-yl)-2-methylbenzo[d][1,3]dioxol-4-yl)piperidin-1-yl)methyl)-1-(thiazol-5-ylmethyl)-1H-benzo[d]imidazole-6-carboxylate 2 (270 mg, 402 μmol, 1 eq) in DMF (2.7 mL) was added 2,2-dimethyl-1,3-dioxane-4,6-dione (289 mg, 2.01 mmol, 5 eq) and Pd(PPh₃)₄ (46.4 mg, 40.2 μmol, 0.1 eq). The mixture was stirred at 50° C. for 12 h. LCMS showed starting material was consumed completely and one main peak with desired m/z was detected. The reaction mixture was diluted with H₂O 10 mL and extracted with ethyl acetate (10 mL*3). The combined organic layers were washed with brine 10 mL, dried over Na₂SO₄, filtered, and concentrated under reduced pressure to give a residue. The residue was purified by column chromatography (SiO₂, Petroleum ether/Ethyl acetate=1/1 to 0/1) to provide product (200 mg, 316 μmol, 79% yield) as a white solid.

LCMS: RT=1.361 min, MS cal.: 631.2, [M+H]⁺=632.0

### Preparation of (S)-2-((4-(2-(5-Chloropyridin-2-yl)-2-methylbenzo[d][1,3]dioxol-4-yl)piperidin-1-yl)methyl)-4-hydroxy-1-(thiazol-5-ylmethyl)-1H-benzo[d]imidazole-6-carboxylic acid, Compound 90 (4)

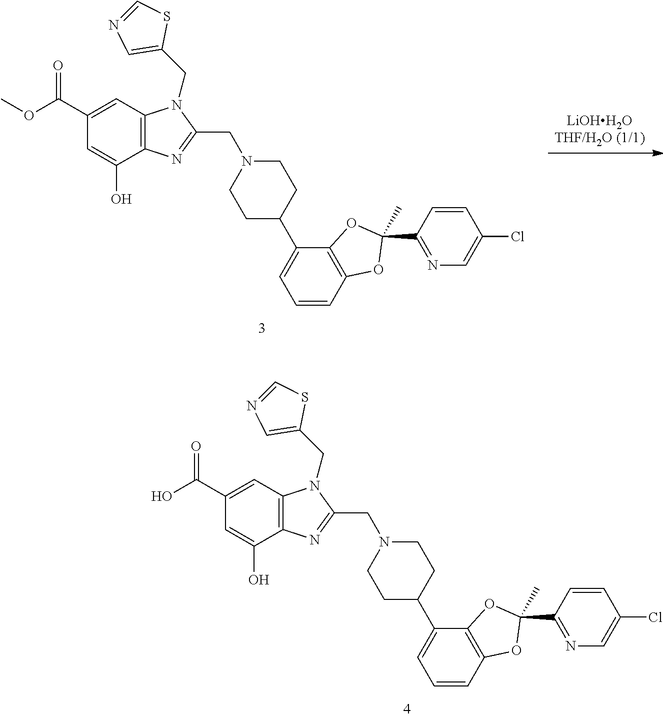 [SMILES: COC(=O)c1cc(O)c2nc(CN3CCC(c4cccc5c4O[C@@](C)(c4ccc(Cl)cn4)O5)CC3)n(Cc3cncs3)c2c1 ; C[C@]1(c2ccc(Cl)cn2)Oc2cccc(C3CCN(Cc4nc5c(O)cc(C(=O)O)cc5n4Cc4cncs4)CC3)c2O1]

To a solution of methyl (S)-2-((4-(2-(5-chloropyridin-2-yl)-2-methylbenzo[d][1,3]dioxol-4-yl)piperidin-1-yl)methyl)-4-hydroxy-1-(thiazol-5-ylmethyl)-1H-benzo[d]imidazole-6-carboxylate 3 (150 mg, 237 μmol, 1 eq) in THF (0.7 mL) was added LiOH·H₂O (19.9 mg, 475 μmol, 2 eq) in H₂O (0.3 mL). The mixture was stirred at 25° C. for 12 h. TLC indicated starting material was consumed completely and one new spot formed. The reaction mixture was concentrated under reduced pressure to remove solvent. The residue was purified by prep-HPLC (column: Waters Xbridge Prep OBD C18 150*40 mm*10 um; mobile phase: [H₂O (10 mM NH₄HCO₃)—CH₃CN]; gradient: 25%-55% B over 8.0 min) to provide product (25.8 mg, 41.6 μmol, 17% yield, 99.5% purity) as a white solid.

LCMS: RT=2.239 min, MS cal.: 617.1, [M+H]⁺=618.2

HPLC: RT=9.753 min, purity: 99.51%

¹H NMR (400 MHz, DMSO-d₆) δ=8.96 (s, 1H), 8.72 (d, J=2.4 Hz, 1H), 8.04-7.97 (m, 2H), 7.64-7.55 (m, 2H), 7.18 (d, J=1.2 Hz, 1H), 6.84-6.74 (m, 2H), 6.73-6.68 (m, 1H), 5.86 (s, 2H), 3.84 (s, 2H), 2.99-2.97 (m, 2H), 2.69-2.61 (m, 1H), 2.23-2.11 (m, 2H), 2.00 (s, 3H), 1.78-1.59 (m, 4H).

### Example A31. —Preparation of Compound 63. (S)-2-((4-(3-(4-cyano-2-fluorophenyl)-2,3-dihydrobenzo[b][1,4]dioxin-5-yl)piperidin-1-yl)methyl)-4-methoxy-1-(thiazol-5-ylmethyl)-1H-benzo[d]imidazole-6-carboxylic acid (Compound 63) (10)

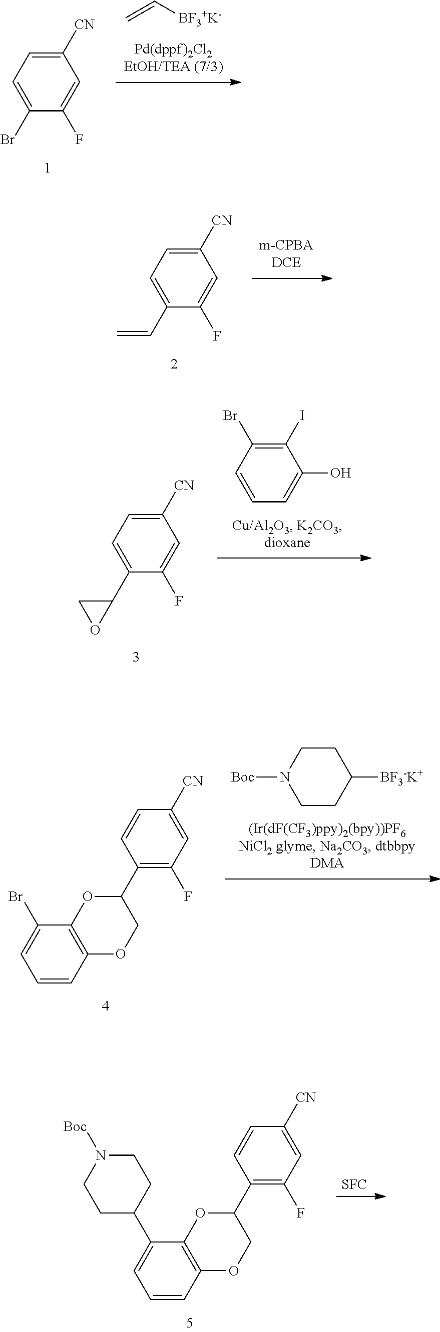

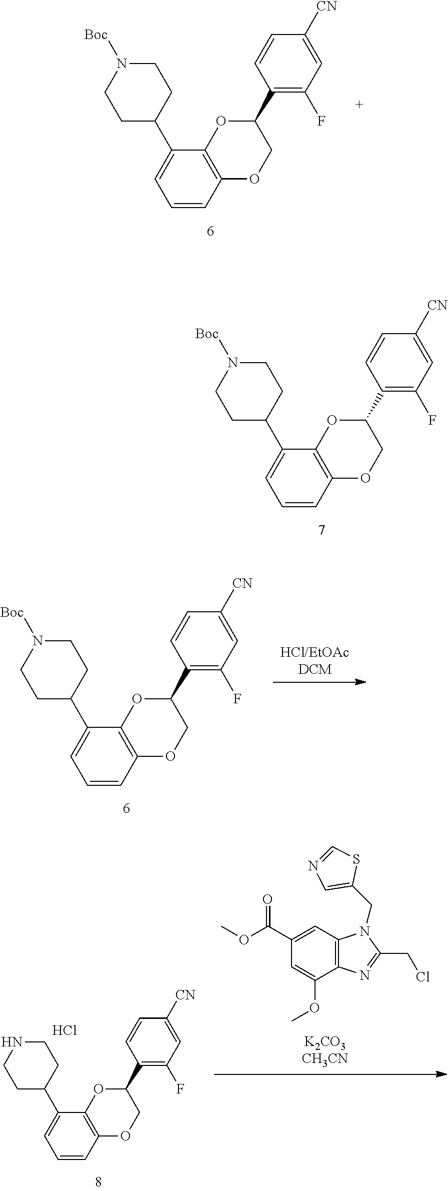

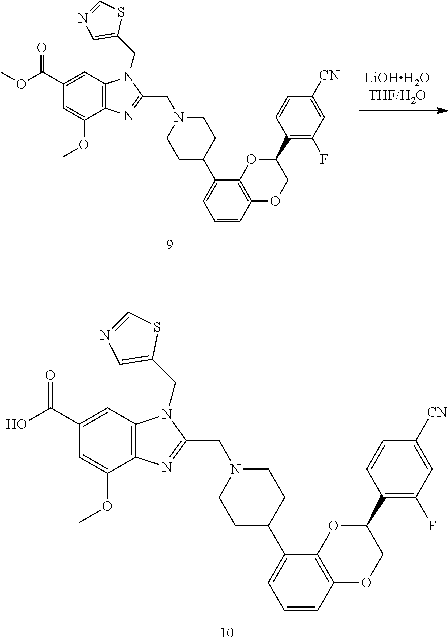 [SMILES: COC(=O)c1cc(OC)c2nc(CN3CCC(c4cccc5c4O[C@@H](c4ccc(C#N)cc4F)CO5)CC3)n(Cc3cncs3)c2c1 ; COc1cc(C(=O)O)cc2c1nc(CN1CCC(c3cccc4c3O[C@@H](c3ccc(C#N)cc3F)CO4)CC1)n2Cc1cncs1]

### Preparation of Intermediate 3-fluoro-4-vinylbenzonitrile (2)

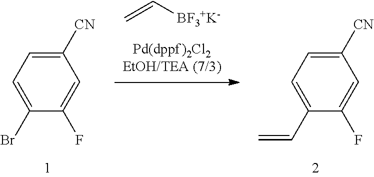

To a solution of 4-bromo-3-fluorobenzonitrile 1 (15 g, 75.00 mmol, 1 eq) and potassium vinyltrifluoroborate (15.5 g, 90 mmol, 1.2 eq, K*) in TEA (45 mL) and EtOH (105 mL) was added Pd(dppf)Cl₂ (1.10 g, 1.50 mmol, 0.02 eq). The mixture was stirred at 100° C. for 4 h. TLC (Petroleum ether/Ethyl acetate=5/1) indicated starting material was consumed completely and one new spot formed. The reaction was clean according to TLC. The reaction mixture was partitioned between ethyl acetate 200 mL and H₂O 150 mL. The organic phase was separated, washed with brine 80 mL (40 mL*2), dried over Na₂SO₄, filtered, and concentrated under reduced pressure to give a residue. The residue was purified by column chromatography (SiO₂, Petroleum ether/Ethyl acetate=100/0 to 90/10) to provide product (9.85 g, 66.9 mmol, 89% yield) as a white solid.

HPLC: RT=2.239 min, purity: 88.61%

1H NMR (400 MHz, CHLOROFORM-d) δ=7.60 (t, J=7.6 Hz, 1H), 7.45-7.40 (d, J=8.4 Hz, 1H), 7.35 (dd, J=1.6, 10.0 Hz, 1H), 6.88 (dd, J=11.2, 18.0 Hz, 1H), 5.97 (d, J=18.0 Hz, 1H), 5.58 (d, J=11.2 Hz, 1H).

### Preparation of Intermediate 3-Fluoro-4-(oxiran-2-yl)benzonitrile (3)

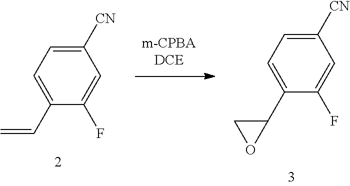

Three reactions were performed in parallel. Equip a 500 mL three-necked round bottom flask, thermometer and N₂ balloon. DCE (300 mL) was charged to the flask, then 3-fluoro-4-vinylbenzonitrile 2 (5.5 g, 37.38 mmol, 1 eq) was added at 20° C. within 3 min. m-CPBA (24.2 g, 112 mmol, 80% purity, 3 eq) was added to the reaction mixture at 0° C. in portions within 5 min. After the addition, the mixture was stirred at 50° C. for 12 h. The reaction was monitored by TLC (Petroleum ether/Ethyl acetate=8/1) indicated starting material was consumed completely and one new spot formed. The reaction was clean according to TLC. After 12 h, the combined reaction mixture was added to sat. Na₂S₂O₃ 100 mL at 0° C. The aqueous layer was neutralized by sat. NaHCO₃ at −5° C.˜5° C. to adjust pH=8-9. The mixture was extracted with DCM (400 mL*3). Then organic phase was washed by H₂O (250 mL), brine (200 mL). The organic phases were dried over Na₂SO₄, filtered. The organic phase was concentrated under reduced pressure at 45° C. to give a residue. The residue was purified by column chromatography (SiO₂, Petroleum ether/Ethyl acetate=100/0 to 90/10) to provide product (13.8 g, 84.59 mmol, 75.43% yield) as a white solid.

HPLC: RT=1.624 min, purity: 96.87%

¹H NMR (400 MHz, CHLOROFORM-d) δ=7.46 (d, J=8.0 Hz, 1H), 7.37 (d, J=9.6 Hz, 1H), 7.35-7.29 (m, 1H), 4.18-4.16 (m, 1H), 3.26-3.23 (m, 1H), 2.77-2.74 (m, 1H).

### Preparation of Intermediate 4-(8-Bromo-2,3-dihydrobenzo[b][1,4]dioxin-2-yl)-3-fluorobenzonitrile (4)

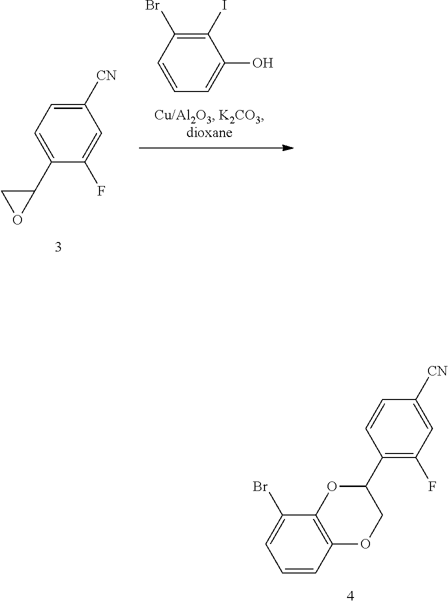 [SMILES: N#Cc1ccc(C2CO2)c(F)c1 ; Oc1cccc(Br)c1I ; N#Cc1ccc(C2COc3cccc(Br)c3O2)c(F)c1]

To a solution of 3-fluoro-4-(oxiran-2-yl)benzonitrile 3 (100 mg, 613 μmol, 1 eq) and 3-bromo-2-iodophenol (183 mg, 613 μmol, 1 eq) in dioxane (1 mL) was added K₂CO₃ (254 mg, 1.84 mmol, 3 eq) and Cu/Al₂O₃ (115 mg, 0.1 eq). The mixture was stirred at 100° C. for 24 h. TLC (Petroleum ether/Ethyl acetate=3/1) indicated starting material was consumed completely and one new spot formed. The aqueous layer was neutralized by FA (0.5 M) at −5° C.˜5° C. to adjust pH=4-5. The reaction mixture was partitioned between MTBE 10 mL and H₂O 25 mL, then the mixture was extracted with MTBE (20 mL*3). The organic phase was separated, washed with brine (5 mL), dried over Na₂SO₄, filtered, and concentrated under reduced pressure to give a residue. The residue was purified by prep-HPLC (column: Agela Dura Shell C18 250*70 mm*10 um; mobile phase: [H₂O (10 mM NH₄HCO₃)—CH₃CN]; gradient: 45%-75% B over 17.0 min) to provide product (1 g, 2.99 mmol, 20% yield) as a white solid.

HPLC: RT=2.811 min, purity: 93.33%

¹H NMR (400 MHz, CHLOROFORM-d) δ=7.76 (t, J=7.6 Hz, 1H), 7.60-7.55 (m, 1H), 7.46-7.41 (m, 1H), 7.22-7.16 (m, 1H), 6.92 (dd, J=1.2, 8.4 Hz, 1H), 6.85-6.77 (m, 1H), 5.59-5.56 (m, 1H), 4.54-4.50 (m, 1H), 3.99-3.95 (m, 1H).

### Preparation of Intermediate tert-Butyl 4-(3-(4-cyano-2-fluorophenyl)-2,3-dihydrobenzo[b][1,4]dioxin-5-yl)piperidine-1-carboxylate (5)

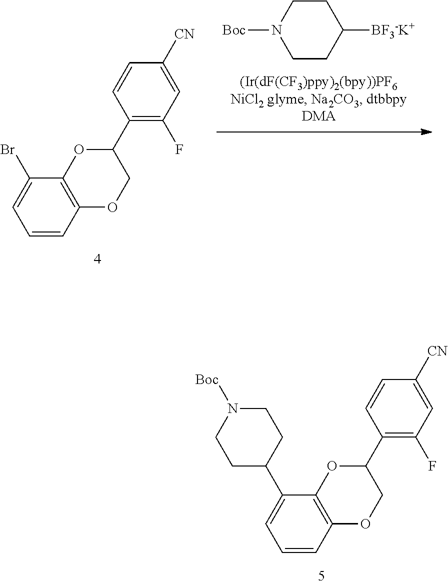

To a solution of 4-(8-bromo-2,3-dihydrobenzo[b][1,4]dioxin-2-yl)-3-fluorobenzonitrile 4 (500 mg, 1.50 mmol, 1 eq) and potassium (1-(tert-butoxycarbonyl)piperidine-4-yl) trifluoroborate (871 mg, 2.99 mmol, 2 eq) in DMA (5 mL) was added NiCl₂ glyme (16.4 mg, 74.8 μmol, 0.05 eq), Na₂CO₃ (317 mg, 2.99 mmol, 2 eq), dtbbpy (20.1 mg, 74.8 μmol, 0.05 eq) and (Ir(dF(CF₃)ppy)₂(bpy))PF₆ (45.3 mg, 44.9 μmol, 0.03 eq). The mixture was stirred at 20° C. for 12 hr. LC-MS showed starting material was consumed completely and one main peak with desired m/z was detected. TLC (Petroleum ether/Ethyl acetate=3/1) indicated starting material was consumed completely and one new spot formed. The reaction was clean according to TLC. The reaction mixture was partitioned between ethyl acetate 2 mL and H₂O 5 mL, the aqueous phase was extracted with ethyl acetate (5 mL*3). The organic phase was combined, washed with brine (3 mL), dried over Na₂SO₄, filtered, and concentrated under reduced pressure to give a residue. The residue was purified by column chromatography (SiO₂, Petroleum ether/Ethyl acetate=100/0 to 93/7) to provide product (400 mg, 912 μmol, 61% yield) as a white solid.

LCMS: RT=2.602 min, MS cal.: 438.49, [M−55]⁺=383.2

¹H NMR (400 MHz, CHLOROFORM-d) δ=7.65-7.59 (m, 1H), 7.58-7.55 (m, 1H), 7.45 (dd, J=1.2, 9.6 Hz, 1H), 6.93-6.88 (m, 1H), 6.85-6.80 (m, 2H), 5.50-5.50 (m, 1H), 4.48-4.45 (m, 1H), 4.27-4.24 (m, 2H), 4.05-3.95 (m, 1H), 3.11-3.01 (m, 1H), 2.88-2.73 (m, 2H), 1.90-1.76 (m, 2H), 1.72-1.59 (m, 2H), 1.47 (s, 9H).

### Preparation of Intermediate tert-butyl(S)-4-(3-(4-cyano-2-fluorophenyl)-2,3-dihydrobenzo[b][1,4]dioxin-5-yl)piperidine-1-carboxylate (6) and Intermediate tert-butyl (R)-4-(3-(4-cyano-2-fluorophenyl)-2,3-dihydrobenzo[b][1,4]dioxin-5-yl)piperidine-1-carboxylate (7)

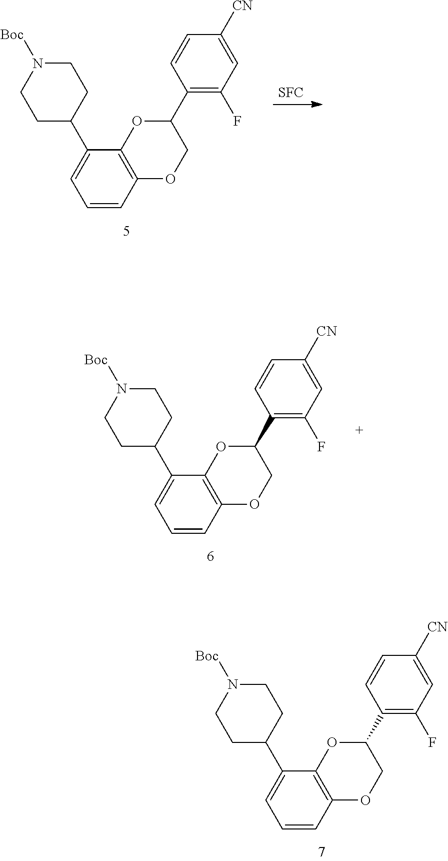

tert-Butyl 4-(3-(4-cyano-2-fluorophenyl)-2,3-dihydrobenzo[b][1,4]dioxin-5-yl)piperidine-1-carboxylate 5 was separated by SFC (column: Chiral Pak IH, 250*30 mm, 10 um; mobile phase: [CO₂-IPA]; B %: 33%, isocratic elution mode).

tert-Butyl(S)-4-(3-(4-cyano-2-fluorophenyl)-2,3-dihydrobenzo[b][1,4]dioxin-5-yl)piperidine-1-carboxylate (6) (441 mg, 1.01 mmol, 67% yield) was obtained as a white solid.

tert-Butyl (R)-4-(3-(4-cyano-2-fluorophenyl)-2,3-dihydrobenzo[b][1,4]dioxin-5-yl)piperidine-1-carboxylate (7) (589 mg, 1.34 mmol, 90% yield) was obtained as a white solid.

¹H NMR (400 MHz, CHLOROFORM-d) δ=7.65-7.59 (m, 1H), 7.59-7.55 (m, 1H), 7.47-7.42 (m, 1H), 6.93-6.87 (m, 1H), 6.86-6.79 (m, 2H), 5.54-5.48 (m, 1H), 4.49-4.46 (m, 1H), 4.29-4.20 (m, 2H), 4.00-3.97 (m, 1H), 3.11-3.01 (m, 1H), 2.80-2.77 (m, 2H), 1.90-1.76 (m, 2H), 1.71-1.59 (m, 2H), 1.46 (s, 9H)

¹H NMR (400 MHz, CHLOROFORM-d) δ=7.67-7.54 (m, 2H), 7.45 (dd, J=1.4, 9.6 Hz, 1H), 6.94-6.77 (m, 3H), 5.53-5.50 (m, 1H), 4.49-4.46 (m, 1H), 4.24-4.20 (m, 2H), 4.00-3.97 (m, 1H), 3.13-3.01 (m, 1H), 2.80-2.77 (m, 2H), 1.90-1.74 (m, 2H), 1.72-1.59 (m, 2H), 1.44 (s, 9H)

### Preparation of Intermediate (8)

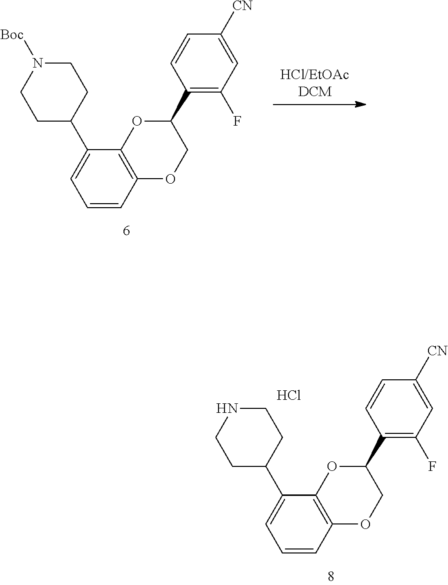 [SMILES: CC(C)(C)OC(=O)N1CCC(c2cccc3c2O[C@@H](c2ccc(C#N)cc2F)CO3)CC1 ; "HCl/EtOAcDCM" ; N#Cc1ccc([C@H]2COc3cccc(C4CCNCC4)c3O2)c(F)c1 ; Cl]

To a solution of tert-butyl(S)-4-(3-(4-cyano-2-fluorophenyl)-2,3-dihydrobenzo[b][1,4]dioxin-5-yl)piperidine-1-carboxylate 6 (175 mg, 399 μmol, 1 eq) in DCM (4 mL) was added HCl/Ethyl acetate (4 M, 4 mL). The mixture was stirred at 20° C. for 2 h. The reaction mixture was concentrated under reduced pressure to provide product (130 mg, crude, HCl) as a white solid.

LCMS: RT=0.376 min, MS cal.: 338.37, [M+H]⁺=339.1.

### Preparation of Intermediate Methyl (S)-2-((4-(3-(4-cyano-2-fluorophenyl)-2,3-dihydrobenzo[b][1,4]dioxin-5-yl)piperidin-1-yl)methyl)-4-methoxy-1-(thiazol-5-ylmethyl)-1H-benzo[d]imidazole-6-carboxylate (9)

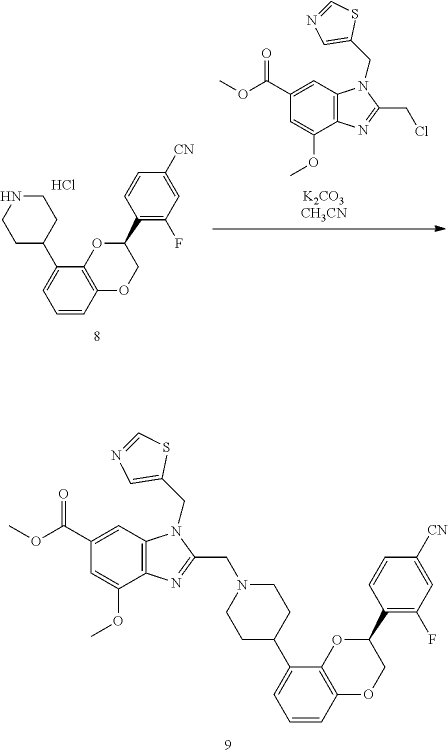 [SMILES: N#Cc1ccc([C@H]2COc3cccc(C4CCNCC4)c3O2)c(F)c1 ; Cl ; COC(=O)c1cc(OC)c2nc(CCl)n(Cc3cncs3)c2c1 ; "K2CO3CH3CN" ; COC(=O)c1cc(OC)c2nc(CN3CCC(c4cccc5c4O[C@@H](c4ccc(C#N)cc4F)CO5)CC3)n(Cc3cncs3)c2c1]

To a solution of methyl 2-(chloromethyl)-4-methoxy-1-(thiazol-5-ylmethyl)-1H-benzo[d]imidazole-6-carboxylate 8 (62.4 mg, 177 μmol, 1 eq) in CH₃CN (0.6 mL) was added K₂CO₃ (73.52 mg, 531.95 μmol, 3 eq) and(S)-3-fluoro-4-(8-(piperidin-4-yl)-2,3-dihydrobenzo[b][1,4]dioxin-2-yl)benzonitrile (60 mg, 177 μmol, 1 eq, HCl). The mixture was stirred at 60° C. for 2 h. The reaction mixture was diluted with H₂O 5 mL and extracted with ethyl acetate 15 mL (5 mL*3). The combined organic layers were washed with brine 10 mL (5 mL*2), dried over Na₂SO₄, filtered, and concentrated under reduced pressure to give a residue. The residue was purified by column chromatography (SiO₂, Petroleum ether/Ethyl acetate=I/O to 0/1) to provide product (40 mg, 61.2 μmol, 35% yield) as a white solid.

LCMS: RT=0.475 min, MS cal.: 653.2/654.2, [M+H]⁺=654.2/655.1

### Preparation of Compound 63, (S)-2-((4-(3-(4-Cyano-2-fluorophenyl)-2,3-dihydrobenzo[b][1,4]dioxin-5-yl)piperidin-1-yl)methyl)-4-methoxy-1-(thiazol-5-ylmethyl)-1H-benzo[d]imidazole-6-carboxylic acid (10)

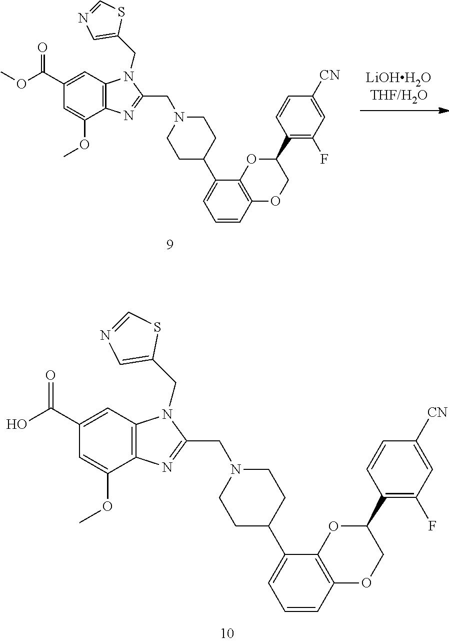 [SMILES: COC(=O)c1cc(OC)c2nc(CN3CCC(c4cccc5c4O[C@@H](c4ccc(C#N)cc4F)CO5)CC3)n(Cc3cncs3)c2c1 ; COc1cc(C(=O)O)cc2c1nc(CN1CCC(c3cccc4c3O[C@@H](c3ccc(C#N)cc3F)CO4)CC1)n2Cc1cncs1]

To a solution of methyl (S)-2-((4-(3-(4-cyano-2-fluorophenyl)-2,3-dihydrobenzo[b][1,4]dioxin-5-yl)piperidin-1-yl)methyl)-4-methoxy-1-(thiazol-5-ylmethyl)-1H-benzo[d]imidazole-6-carboxylate 9 (70 mg, 107 μmol, 1 eq) in THF (0.7 mL) was added LiOH·H₂O (4.49 mg, 107 μmol, 1 eq) in H₂O (0.3 mL). The mixture was stirred at 20° C. for 12 h. LCMS showed starting material remained. Then LiOH·H₂O (1.35 mg, 32.1 μmol, 0.3 eq) was added to the mixture. The mixture was stirred at 20° C. for 2 h. LCMS showed ˜35% of starting material remained and ˜49% of desired compound was detected. The reaction mixture was diluted with H₂O 1 mL. The mixture was purified by prep-HPLC (neutral condition: column: Waters Xbridge Prep OBD C18 150*40 mm*10 um; mobile phase: [H₂O (10 mM NH₄HCO₃)—CH₃CN]; gradient: 35%-65% B over 8.0 min) to provide product (11.3 mg, 8.19 μmol, 7.7% yield, 99.6% purity) as a white solid.

LCMS: RT=2.315 min, MS cal.: 640.2, [M+H]⁺=641.2

HPLC: RT=10.387 min, purity: 99.59%

¹H NMR (400 MHz, DMSO-d₆) δ=9.02 (s, 1H), 8.03-7.94 (m, 2H), 7.82-7.80 (m, 2H), 7.72-7.62 (m, 1H), 7.27 (s, 1H), 6.91-6.82 (m, 1H), 6.80-6.78 (m, 2H), 5.94 (s, 2H), 5.63-5.54 (m, 1H), 4.52-4.42 (m, 1H), 4.15-4.06 (m, 1H), 3.94 (s, 3H), 3.88-3.76 (m, 2H), 2.95-2.80 (m, 3H), 2.19-2.08 (m, 2H), 1.79-1.70 (m, 1H), 1.69-1.51 (m, 3H)

### Example A32. —Preparation of Compound 91. (R)-2-((4-(3-(4-cyano-2-fluorophenyl)-2,3-dihydrobenzo[b][1,4]dioxin-5-yl)piperidin-1-yl)methyl)-4-methoxy-1-(thiazol-5-ylmethyl)-1H-benzo[d]imidazole-6-carboxylic acid (Compound 91) (4)

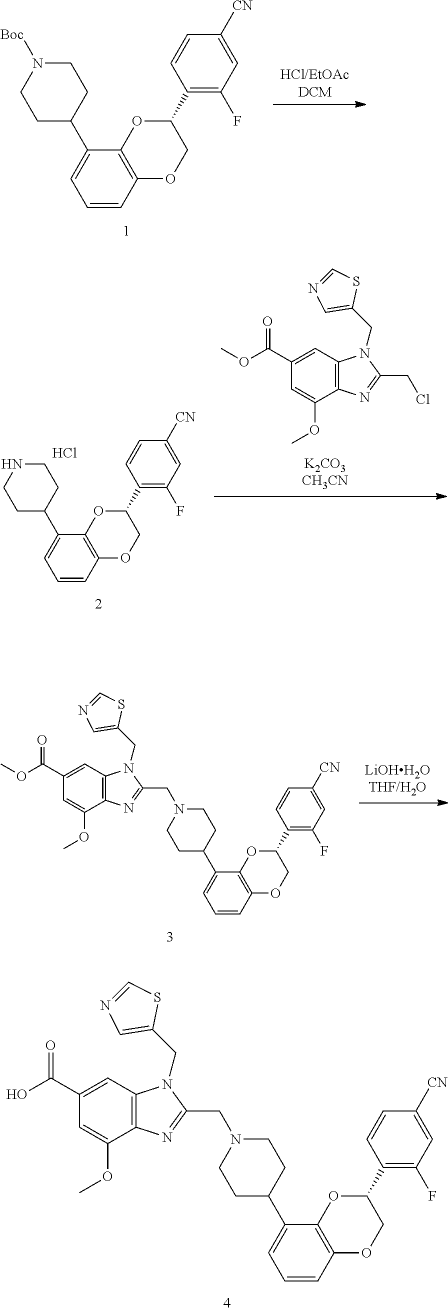

### Preparation of Intermediate (R)-3-fluoro-4-(8-(piperidin-4-yl)-2,3-dihydrobenzo[b][1,4]dioxin-2-yl)benzonitrile (2)

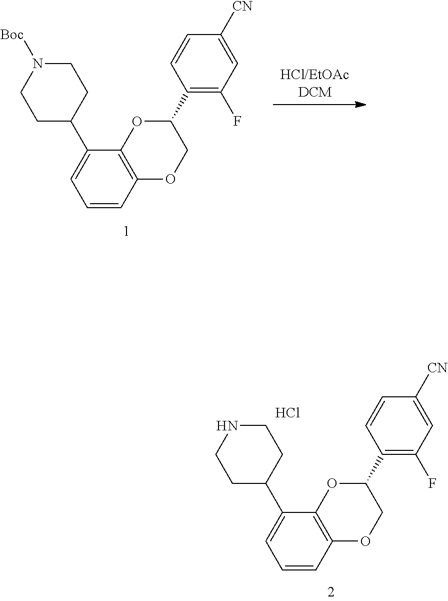 [SMILES: CC(C)(C)OC(=O)N1CCC(c2cccc3c2O[C@H](c2ccc(C#N)cc2F)CO3)CC1 ; "HCl/EtOAcDCM" ; N#Cc1ccc([C@@H]2COc3cccc(C4CCNCC4)c3O2)c(F)c1 ; Cl]

A mixture of tert-butyl (R)-4-(3-(4-cyano-2-fluorophenyl)-2,3-dihydrobenzo[b][1,4]dioxin-5-yl)piperidine-1-carboxylate 1 (0.16 g, 365 μmol, 1 eq) in DCM (4 mL) HCl/Ethyl acetate (4 M, 4 mL) was stirred at 25° C. for 2 h. LCMS showed starting material was consumed completely and one main peak with desired m/z was detected. The reaction mixture was concentrated under reduced pressure to provide product (129 mg, crude, HCl) as a yellow solid.

### Preparation of Intermediate Methyl (R)-2-((4-(3-(4-cyano-2-fluorophenyl)-2,3-dihydrobenzo[b][1,4]dioxin-5-yl)piperidin-1-yl)methyl)-4-methoxy-1-(thiazol-5-ylmethyl)-1H-benzo[d]imidazole-6-carboxylate (3)

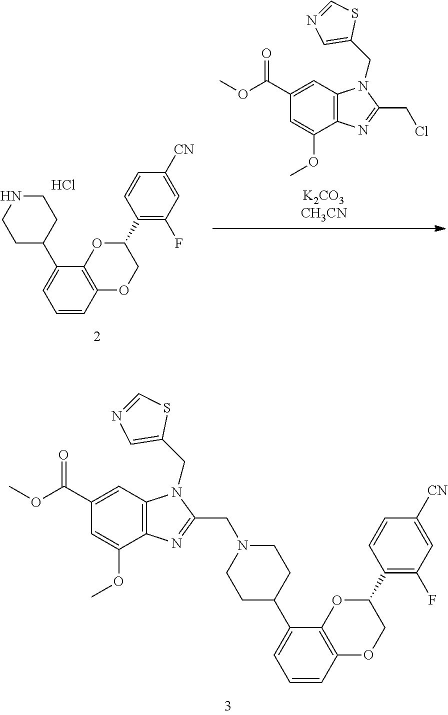 [SMILES: N#Cc1ccc([C@@H]2COc3cccc(C4CCNCC4)c3O2)c(F)c1 ; Cl ; COC(=O)c1cc(OC)c2nc(CCl)n(Cc3cncs3)c2c1 ; "K2CO3CH3CN" ; COC(=O)c1cc(OC)c2nc(CN3CCC(c4cccc5c4O[C@H](c4ccc(C#N)cc4F)CO5)CC3)n(Cc3cncs3)c2c1]

A mixture of methyl 2-(chloromethyl)-4-methoxy-1-(thiazol-5-ylmethyl)-1H-benzo[d]imidazole-6-carboxylate (100 mg, 284 μmol, 1 eq), (R)-3-fluoro-4-(8-(piperidin-4-yl)-2,3-dihydrobenzo[b][1,4]dioxin-2-yl)benzonitrile 2 (67.3 mg, 199 μmol, 0.7 eq, HCl) and K₂CO₃ (118 mg, 853 μmol, 3 eq) in CH₃CN (1 mL) was degassed and purged with N₂ for 3 times, and then the mixture was stirred at 60° C. for 2 h under N₂ atmosphere. The reaction mixture was quenched by H₂O 5 mL at 20° C., extracted with ethyl acetate 15 mL (5 mL*3). The combined organic layers were washed with brine 10 mL (5 mL*2), dried over Na₂SO₄, filtered, and concentrated under reduced pressure to give a residue. The residue was purified by column chromatography (SiO₂, Petroleum ether/Ethyl acetate=80/1 to 20/1) to provide product (100 mg, 136 μmol, 48% yield, 89% purity) as a white solid.

SFC: ee %=96.82%

### Preparation of (R)-2-((4-(3-(4-Cyano-2-fluorophenyl)-2,3-dihydrobenzo[b][1,4]dioxin-5-yl)piperidin-1-yl)methyl)-4-methoxy-1-(thiazol-5-ylmethyl)-1H-benzo[d]imidazole-6-carboxylic acid, Compound 91 (4)

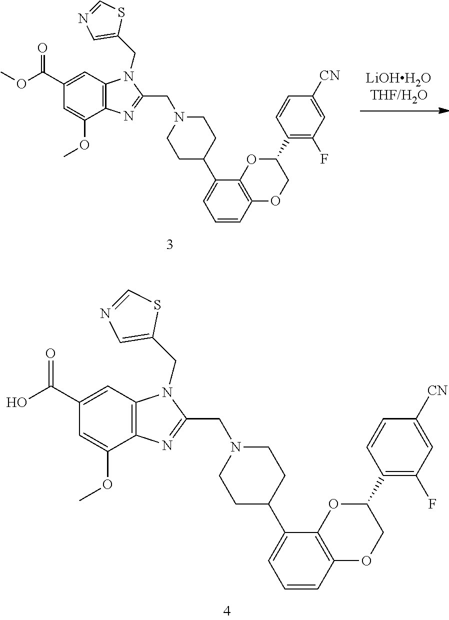 [SMILES: COC(=O)c1cc(OC)c2nc(CN3CCC(c4cccc5c4O[C@H](c4ccc(C#N)cc4F)CO5)CC3)n(Cc3cncs3)c2c1 ; COc1cc(C(=O)O)cc2c1nc(CN1CCC(c3cccc4c3O[C@H](c3ccc(C#N)cc3F)CO4)CC1)n2Cc1cncs1]

A mixture of methyl (R)-2-((4-(3-(4-cyano-2-fluorophenyl)-2,3-dihydrobenzo[b][1,4]dioxin-5-yl)piperidin-1-yl)methyl)-4-methoxy-1-(thiazol-5-ylmethyl)-1H-benzo[d]imidazole-6-carboxylate 3 (100 mg, 153 μmol, 1 eq) in THF (0.7 mL) was added LiOH·H₂O (7.06 mg, 168 μmol, 1.1 eq) in H₂O (0.3 mL) was degassed and purged with N₂ for 3 times, and then the mixture was stirred at 25° C. for 12 h under N₂ atmosphere. LCMS showed the starting material was consumed completely. The reaction mixture was concentrated under reduced pressure to remove solvent. The residue was purified by prep-HPLC (column: Waters Xbridge Prep OBD C18 150*40 mm*10 um; mobile phase: [H₂O (10 mM NH₄HCO₃)—CH₃CN]; gradient: 10%-60% B over 8.0 min) to provide product (25.0 mg, 38.2 μmol, 25% yield, 97.6% purity) as a white solid.

LCMS: RT=2.623 min, MS cal.: 639.2, [M+H]⁺=640.3

HPLC: RT=10.288 min, purity: 97.63%

¹H NMR (400 MHz, DMSO-d6) δ=9.00 (s, 1H), 8.02-7.95 (m, 2H), 7.85-7.78 (m, 2H), 7.66 (t, J=7.6 Hz, 1H), 6.88-6.82 (m, 1H), 6.81-6.75 (m, 2H), 5.90 (s, 2H), 5.59 (dd, J=2.2, 7.5 Hz, 1H), 4.47 (dd, J=2.4, 11.4 Hz, 1H), 4.11 (dd, J=7.6, 11.6 Hz, 1H), 3.94 (s, 3H), 3.88-3.76 (m, 2H), 2.99-2.80 (m, 3H), 2.23-2.07 (m, 2H), 1.80-1.45 (m, 4H).

### Example A33. —Preparation of Compound 92. 2-((4-((S)-2-(5-Chloropyridin-2-yl)-2-methylbenzo[d][1,3]dioxol-4-yl)piperidin-1-yl)methyl)-4-hydroxy-1-(((S)-oxetan-2-yl)methyl)-1H-benzo[d]imidazole-6-carboxylic acid (7)

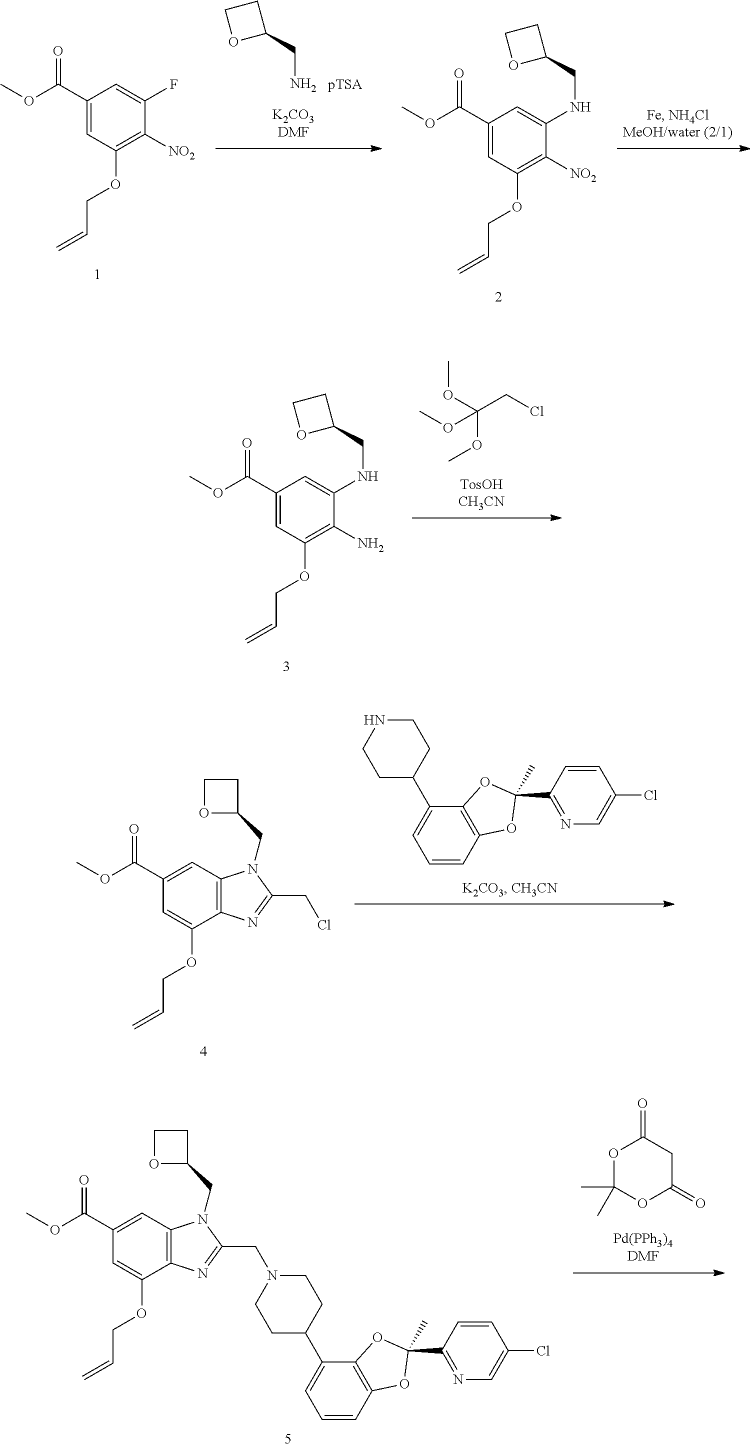

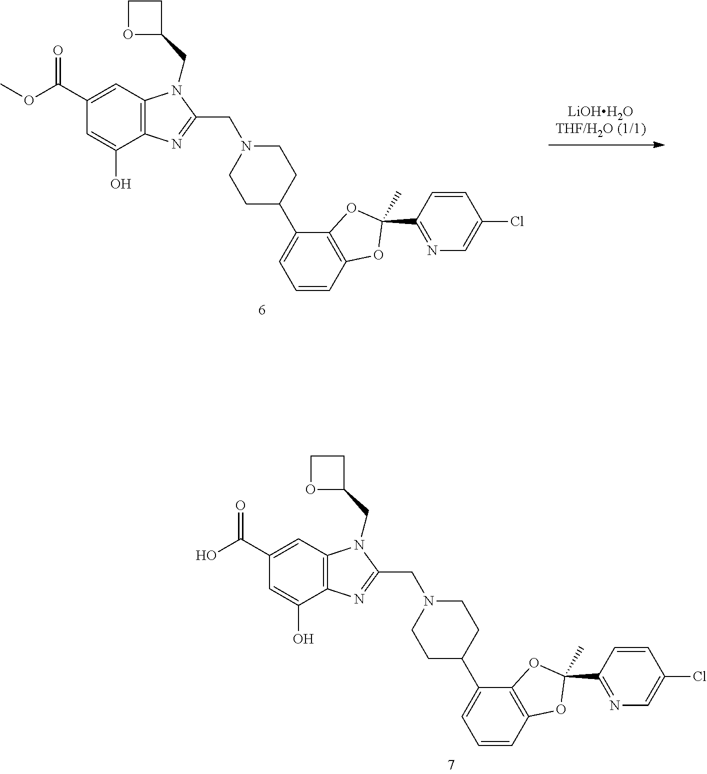 [SMILES: COC(=O)c1cc(O)c2nc(CN3CCC(c4cccc5c4O[C@@](C)(c4ccc(Cl)cn4)O5)CC3)n(C[C@@H]3CCO3)c2c1 ; C[C@]1(c2ccc(Cl)cn2)Oc2cccc(C3CCN(Cc4nc5c(O)cc(C(=O)O)cc5n4C[C@@H]4CCO4)CC3)c2O1]

### Preparation of Intermediate Methyl (S)-3-(allyloxy)-4-nitro-5-((oxetan-2-ylmethyl)amino)benzoate (2)

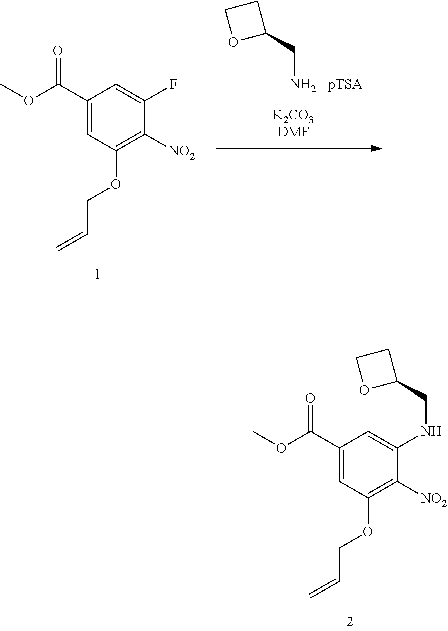 [SMILES: C=CCOc1cc(C(=O)OC)cc(F)c1[N+](=O)[O-] ; NC[C@@H]1CCO1 ; C=CCOc1cc(C(=O)OC)cc(NC[C@@H]2CCO2)c1[N+](=O)[O-]]

A mixture of methyl 3-(allyloxy)-5-fluoro-4-nitrobenzoate 1 (900 mg, 3.53 mmol, 1 eq), (S)-oxetan-2-ylmethanamine (1.37 g, 5.29 mmol, 1.5 eq, p-TSA), K₂CO₃ (1.46 g, 10.6 mmol, 3 eq) in DMF (10 mL) was degassed and purged with N₂ for 3 times, and then the mixture was stirred at 60° C. for 12 h under N₂ atmosphere. LCMS showed starting material was consumed completely. The reaction mixture was diluted with H₂O (20 mL) and extracted with DCM (20 mL*5). The organic phase was dried over Na₂SO₄, filtered, and concentrated under reduced pressure to give a residue. The residue was purified by column chromatography (SiO₂, Petroleum ether/Ethyl acetate=19/1 to 0/1) to provide product (1.1 g, 3.41 mmol, 97% yield) as an orange oil.

LCMS: RT=1.186, MS cal.: 322.1, [M+H]⁺=323.1

¹H NMR (400 MHz, CHLOROFORM-d) δ=7.14 (s, 1H), 6.96 (s, 1H), 6.09-5.94 (m, 1H), 5.47 (qd, J=1.6, 17.2 Hz, 1H), 5.38-5.28 (m, 1H), 5.14-5.01 (m, 1H), 4.78-4.64 (m, 3H), 4.59-4.57 (m, 1H), 3.94 (s, 3H), 3.49 (d, J=4.4 Hz, 2H), 2.80-2.67 (m, 1H), 2.59-2.56 (m, 1H)

### Preparation of Intermediate Methyl (S)-3-(allyloxy)-4-amino-5-((oxetan-2-ylmethyl)amino)benzoate (3)

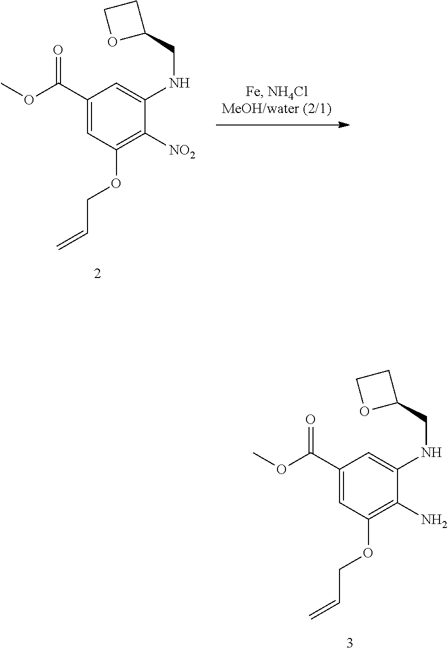 [SMILES: C=CCOc1cc(C(=O)OC)cc(NC[C@@H]2CCO2)c1[N+](=O)[O-] ; C=CCOc1cc(C(=O)OC)cc(NC[C@@H]2CCO2)c1N]

Equip a 50 mL three-necked round bottom flask, N₂ balloon. MeOH (8 mL) and H₂O (4 mL) was charged to the 50 mL three-necked round bottom flask, the mixture was degassed and purged with N₂ for 3 times, then methyl (S)-3-(allyloxy)-4-nitro-5-((oxetan-2-ylmethyl)amino)benzoate 2 (1.1 g, 3.41 mmol, 1 eq) and NH₄Cl (913 mg, 17.1 mmol, 5 eq) was added to the mixture at 20° C. for 10 min. At 20° C. (inner temperature), Fe (953 mg, 17.1 mmol, 5 eq) was added in portions to the reaction mixture at 20° C. within 10 min. After the addition, the mixture was stirred at 70° C. for 1 h under N₂ atmosphere. LCMS showed starting material was consumed completely. The suspension was filtered through a pad of silica gel and the filter cake was washed with MeOH (100 mL). The combined filtrates were concentrated to provide product (0.95 g, 3.25 mmol, 95% yield) as a yellow oil.

LCMS: RT=1.014 min. MS cal.: 292.1, [M+H]⁺=293.1

¹H NMR (400 MHz, CHLOROFORM-d) δ=7.22-7.15 (m, 2H), 6.15-6.00 (m, 1H), 5.44-5.40 (m, 1H), 5.35-5.25 (m, 1H), 5.16-5.03 (m, 1H), 4.75-4.72 (m, 1H), 4.67-4.58 (m, 3H), 3.90 (s, 3H), 3.50-3.44 (m, 1H), 3.41-3.36 (m, 1H), 2.82-2.70 (m, 1H), 2.61-2.59 (m, 1H)

### Preparation of Intermediate Methyl (S)-4-(allyloxy)-2-(chloromethyl)-1-(oxetan-2-ylmethyl)-1H-benzo[d]imidazole-6-carboxylate (4)

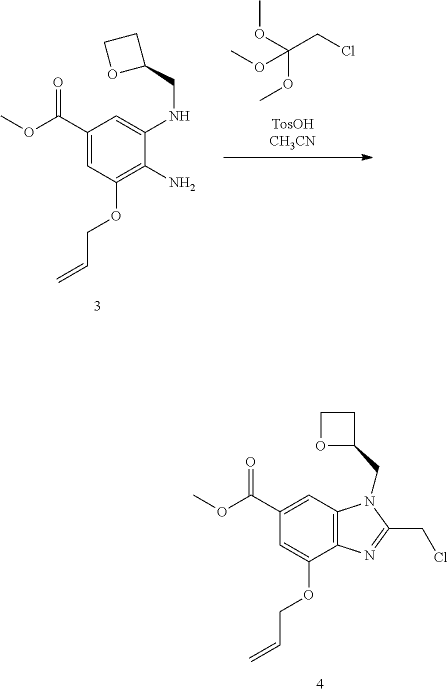

A mixture of methyl (S)-3-(allyloxy)-4-amino-5-((oxetan-2-ylmethyl)amino)benzoate 3 (950 mg, 3.25 mmol, 1 eq), 2-chloro-1,1,1-trimethoxyethane (1.51 g, 9.75 mmol, 1.31 mL, 3 eq), p-TSA (112 mg, 650 μmol, 0.2 eq) in ACN (10 mL) was degassed and purged with N₂ for 3 times, and then the mixture was stirred at 60° C. for 12 h under N₂ atmosphere. LCMS showed starting material was consumed completely. TLC (Petroleum ether/Ethyl acetate=0/1, R_{f}=0.4) indicated starting material was consumed completely. The reaction mixture was diluted with H₂O (20 mL) and extracted with DCM (20 mL*4). The organic phase was dried over Na₂SO₄, filtered, and concentrated under reduced pressure to give a residue. The residue was purified by column chromatography (SiO₂, Petroleum ether/Ethyl acetate=20/1 to 0/1) to provide product (900 mg, 2.57 mmol, 79% yield) as a yellow solid.

LCMS: RT=1.081 min, MS cal.: 350.1, [M+H]⁺=351.0

¹H NMR (400 MHz, CHLOROFORM-d) δ=7.75 (s, 1H), 7.43 (d, J=1.2 Hz, 1H), 6.23-6.16 (m, 1H), 5.49 (qd, J=1.6, 17.2 Hz, 1H), 5.34 (dd, J=1.2, 10.4 Hz, 1H), 5.25-5.18 (m, 1H), 5.12-5.01 (m, 2H), 4.88-4.84 (m, 2H), 4.65-4.53 (m, 3H), 4.32-4.29 (m, 1H), 3.95 (s, 3H), 2.76-2.72 (m, 1H), 2.42-2.37 (m, 1H)

### Preparation of Intermediate Methyl 4-(allyloxy)-2-((4-((S)-2-(5-chloropyridin-2-yl)-2-methylbenzo[d][1,3]dioxol-4-yl)piperidin-1-yl)methyl)-1-(((S)-oxetan-2-yl)methyl)-1H-benzo[d]imidazole-6-carboxylate (5)

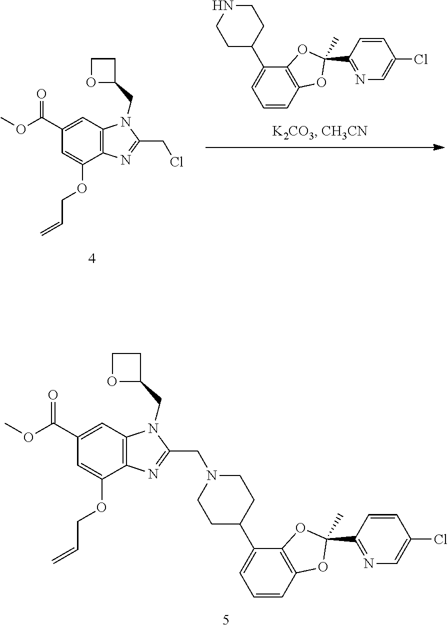 [SMILES: C=CCOc1cc(C(=O)OC)cc2c1nc(CCl)n2C[C@@H]1CCO1 ; C[C@]1(c2ccc(Cl)cn2)Oc2cccc(C3CCNCC3)c2O1 ; C=CCOc1cc(C(=O)OC)cc2c1nc(CN1CCC(c3cccc4c3O[C@@](C)(c3ccc(Cl)cn3)O4)CC1)n2C[C@@H]1CCO1]

A mixture of methyl (S)-4-(allyloxy)-2-(chloromethyl)-1-(oxetan-2-ylmethyl)-1H-benzo[d]imidazole-6-carboxylate 4 (400 mg, 1.14 mmol, 1 eq), (S)-5-chloro-2-(2-methyl-4-(piperidin-4-yl)benzo[d][1,3]dioxol-2-yl)pyridine (415 mg, 1.25 mmol, 1.1 eq), K₂CO₃ (473 mg, 3.42 mmol, 3 eq) in ACN (5 mL) was degassed and purged with N₂ for 3 times, and then the mixture was stirred at 60° C. for 2 h under N₂ atmosphere. TLC (Petroleum ether/Ethyl acetate=0/1, R_{f}=0.3) indicated starting material was consumed completely. The reaction mixture was diluted with H₂O (15 mL) and extracted with DCM (15 mL*4). The organic phase was separated, dried over Na₂SO₄, filtered, and concentrated under reduced pressure to give a residue. The residue was purified by column chromatography (SiO₂, Petroleum ether/Ethyl acetate=20/1 to Petroleum ether/MeOH=0/1) to provide product (400 mg, 620 μmol, 54% yield) as a white solid.

LCMS: RT=1.562 min, MS cal.: 644.2, [M+H]⁺=645.1

### Preparation of Intermediate Methyl 2-((4-((S)-2-(5-chloropyridin-2-yl)-2-methylbenzo[d][1,3]dioxol-4-yl)piperidin-1-yl)methyl)-4-hydroxy-1-(((S)-oxetan-2-yl)methyl)-1H-benzo[d]imidazole-6-carboxylate (6)

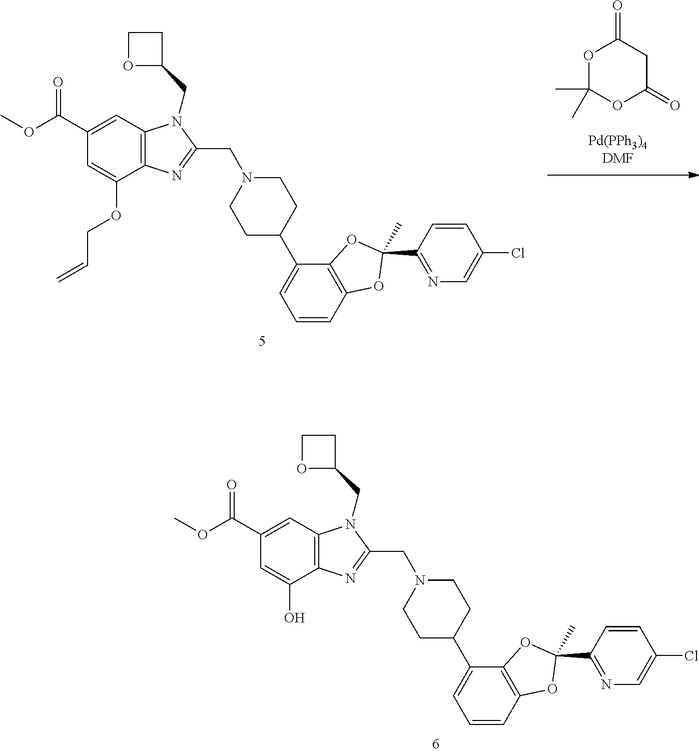

A mixture of methyl 4-(allyloxy)-2-((4-((S)-2-(5-chloropyridin-2-yl)-2-methylbenzo[d][1,3]dioxol-4-yl)piperidin-1-yl)methyl)-1-(((S)-oxetan-2-yl)methyl)-1H-benzo[d]imidazole-6-carboxylate 5 (600 mg, 930 μmol, 1 eq), 2,2-dimethyl-1,3-dioxane-4,6-dione (670 mg, 4.65 mmol, 5 eq), Pd(PPh₃)₄ (107 mg, 93.0 μmol, 0.1 eq) in DMF (6 mL) was degassed and purged with N₂ for 3 times, and then the mixture was stirred at 50° C. for 12 h under N₂ atmosphere. LCMS showed starting material was consumed completely. The reaction mixture was diluted with H₂O (8 mL) and extracted with DCM (8*4 mL). The organic phase was dried over Na₂SO₄, filtered, and concentrated under reduced pressure to give a residue. The residue was purified by prep-HPLC (column: Waters Xbridge BEH C18 250*50 mm*10 um; mobile phase: [H₂O (10 mM NH₄HCO₃)-ACN]; gradient: 30%-80% B over 10.0 min) to provide product (300 mg, 496 μmol, 53% yield) as a white solid.

LCMS: RT=2.227 min, MS cal.: 604.2, [M+H]⁺=605.2

¹H NMR (400 MHz, CHLOROFORM-d) δ=8.61 (d, J=2.0 Hz, 1H), 7.72 (d, J=1.2 Hz, 1H), 7.68 (dd, J=2.4, 8.4 Hz, 1H), 7.60-7.53 (m, 2H), 6.81-6.76 (m, 1H), 6.73-6.68 (m, 2H), 5.27-5.16 (m, 1H), 4.84-4.73 (m, 1H), 4.69-4.58 (m, 2H), 4.44-4.42 (m, 1H), 4.08-3.98 (m, 2H), 3.96 (s, 3H), 3.03-2.88 (m, 2H), 2.79-2.70 (m, 2H), 2.55-2.45 (m, 1H), 2.41-2.28 (m, 2H), 2.05 (s, 3H), 1.96-1.80 (m, 4H)

### Preparation of 2-((4-((S)-2-(5-Chloropyridin-2-yl)-2-methylbenzo[d][1,3]dioxol-4-yl)piperidin-1-yl)methyl)-4-hydroxy-1-(((S)-oxetan-2-yl)methyl)-1H-benzo[d]imidazole-6-carboxylic acid, Compound 92 (7)

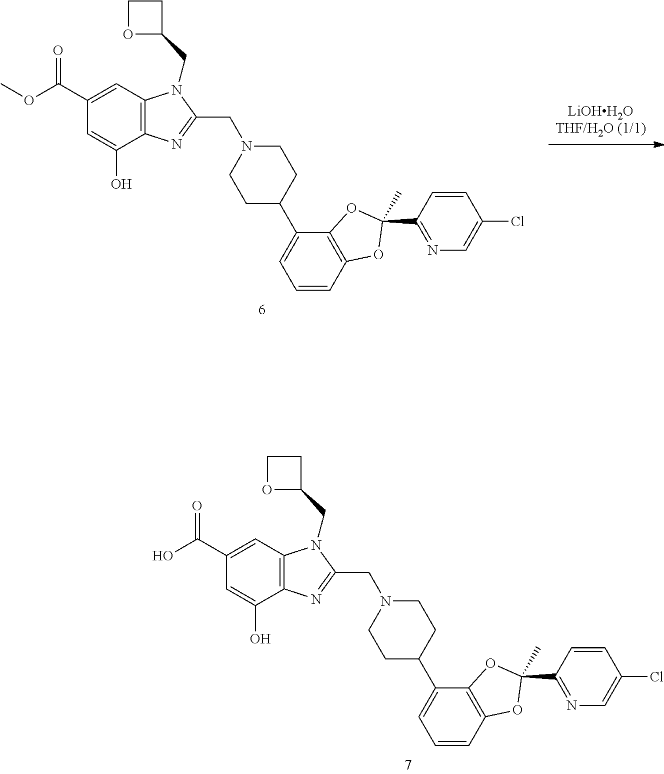 [SMILES: COC(=O)c1cc(O)c2nc(CN3CCC(c4cccc5c4O[C@@](C)(c4ccc(Cl)cn4)O5)CC3)n(C[C@@H]3CCO3)c2c1 ; C[C@]1(c2ccc(Cl)cn2)Oc2cccc(C3CCN(Cc4nc5c(O)cc(C(=O)O)cc5n4C[C@@H]4CCO4)CC3)c2O1]

A mixture of methyl 2-((4-((S)-2-(5-chloropyridin-2-yl)-2-methylbenzo[d][1,3]dioxol-4-yl)piperidin-1-yl)methyl)-4-hydroxy-1-(((S)-oxetan-2-yl)methyl)-1H-benzo[d]imidazole-6-carboxylate 6 (99 mg, 164 μmol, 1 eq), LiOH·H₂O (27.5 mg, 654 μmol, 4 eq) in THF (1 mL) and H₂O (1 mL) was degassed and purged with N₂ for 3 times, and then the mixture was stirred at 25° C. for 12 h under N₂ atmosphere. LCMS showed starting material was consumed completely. The reaction mixture was filtered. The filtrate was purified by prep-HPLC (column: Waters Xbridge Prep OBD C₁₈ 150*40 mm*10 um; mobile phase: [H₂O (10 mM NH₄HCO₃)-ACN]; gradient: 10%-60% B over 8.0 min) to provide product (24.8 mg, 40.68 μmol, 25% yield, 97.2% purity) as a white solid.

LCMS: RT=2.575 min, MS cal.: 590.2, [M+H]⁺=591.2

HPLC: RT=10.038 min, purity: 97.15%

¹H NMR (400 MHz, METHANOL-d₄) δ=8.60 (d, J=2.0 Hz, 1H), 7.88 (dd, J=2.4, 8.4 Hz, 1H), 7.78 (d, J=1.2 Hz, 1H), 7.65 (d, J=8.4 Hz, 1H), 7.34 (d, J=1.2 Hz, 1H), 6.78 (d, J=7.6 Hz, 1H), 6.76-6.69 (m, 2H), 5.26-5.19 (m, 1H), 4.81-4.76 (m, 1H), 4.69-4.59 (m, 2H), 4.49-4.45 (m, 1H), 4.14-4.05 (m, 1H), 4.03-3.96 (m, 1H), 3.23-3.12 (m, 1H), 3.10-3.02 (m, 1H), 2.83-2.71 (m, 2H), 2.57-2.34 (m, 3H), 2.02 (s, 3H), 1.97-1.79 (m, 4H).

### Example A34. —Preparation of Compound 93. 2-((4-((S)-2-(5-Chloropyridin-2-yl)-2-methylbenzo[d][1,3]dioxol-4-yl)piperidin-1-yl)methyl)-3-(((S)-oxetan-2-yl)methyl)-7,8-dihydro-3H-[1,4]dioxino[2′,3′:3,4]benzo[1,2-d]imidazole-5-carboxylic acid (Compound 93) (18)

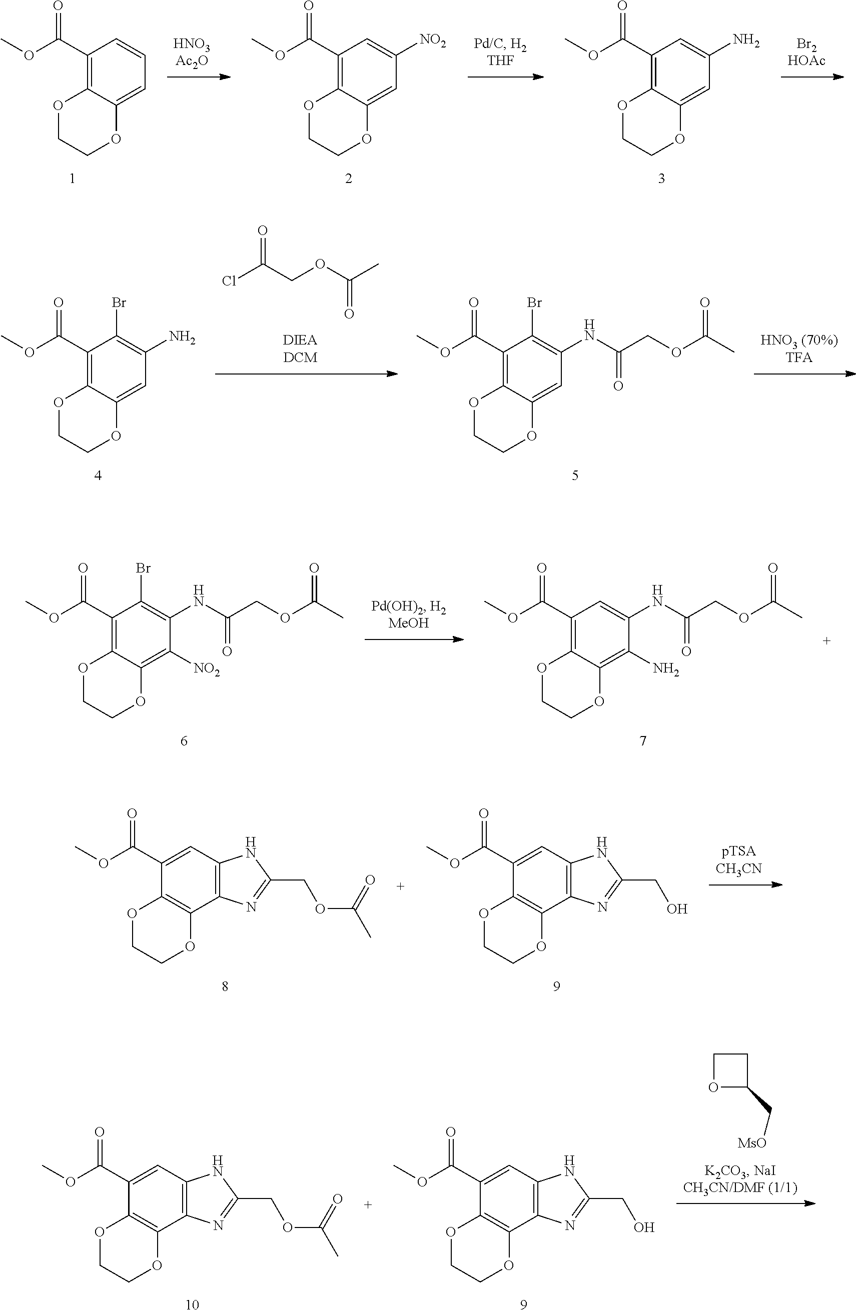

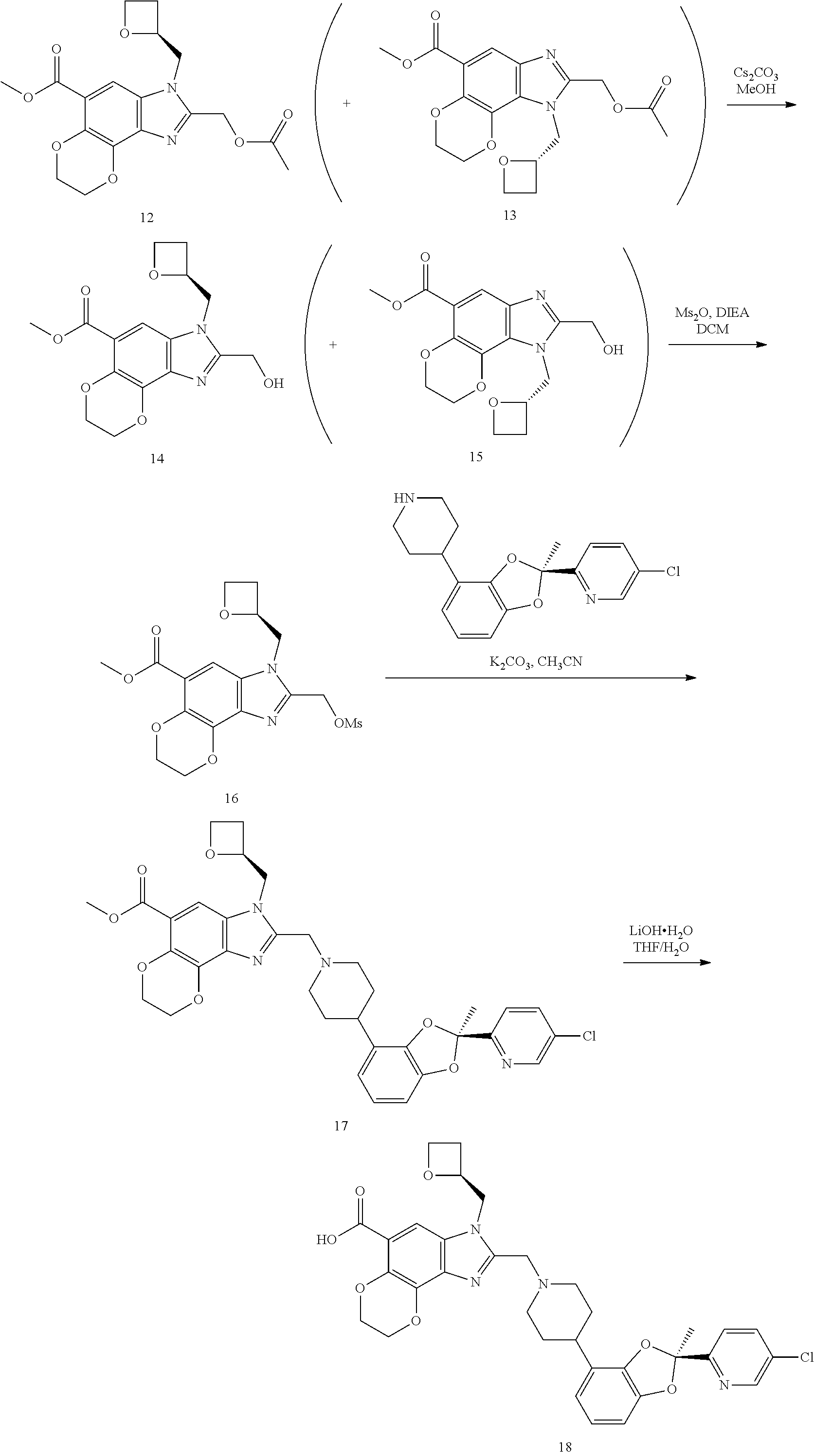

### Preparation of Intermediate Methyl 7-nitro-2,3-dihydrobenzo[b][1,4]dioxine-5-carboxylate (2)

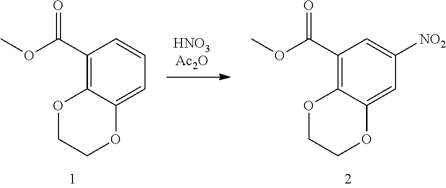 [SMILES: COC(=O)c1cccc2c1OCCO2 ; COC(=O)c1cc([N+](=O)[O-])cc2c1OCCO2]

Equip a 500 mL three-necked round bottom flask and thermometer, N₂ balloon. Ac₂O (91 mL) was charged to the three-necked round bottom flask, and then methyl 2,3-dihydrobenzo[b][1,4]dioxine-5-carboxylate 1 (13 g, 66.9 mmol, 1 eq) was added to the mixture at 15° C. At 15° C. (inner temperature) HNO₃ (6.33 g, 100 mmol, 4.52 mL, 70% purity, 1.5 eq) was added to the reaction mixture at 15° C. within 1 min. After the addition, the mixture was stirred at 15° C. for 1 hr. TLC (Petroleum ether/Ethyl acetate=1/1, R_{f}=0.54) indicated starting material was consumed completely and one new spot formed. The reaction was clean according to TLC. The mixture was poured into a mixture of ice water (50 mL) and extracted by DCM (20 mL*2). The organic phase was added with sat. NaHCO₃ adjust to pH=7˜8, and then washed by brine (25 mL*2) dried over Na₂SO₄, filtered and concentrated. The residue was purified by column chromatography (SiO₂, Petroleum ether/Ethyl acetate=20/1 to 2/1) to provide product (2.2 g, 9.20 mmol, 13% yield) as a white solid.

LCMS: RT=0.678. min, MS cal.: 239.0, [M+H] ·=240.2

¹H NMR (400 MHz, CHLOROFORM-d) δ=8.35 (d, J=2.8 Hz, 1H), 7.92 (d, J=2.8 Hz, 1H), 4.51-4.46 (m, 2H), 4.40-4.34 (m, 2H), 3.94 (s, 3H)

### Preparation of Intermediate Methyl 7-amino-2,3-dihydrobenzo[b][1,4]dioxine-5-carboxylate (3)

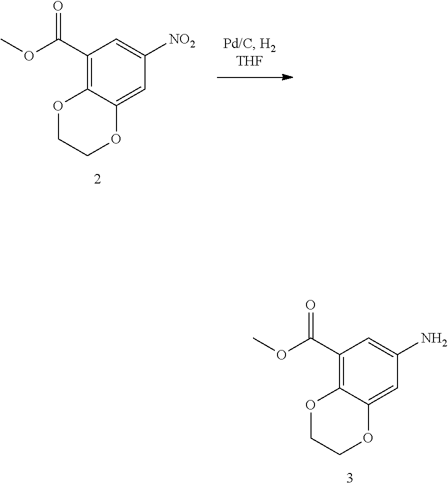 [SMILES: COC(=O)c1cc([N+](=O)[O-])cc2c1OCCO2 ; COC(=O)c1cc(N)cc2c1OCCO2]

Equip a 250 mL round bottom flask, H₂ atmosphere. THF (25 mL) was charged to the round bottom flask, then methyl 7-nitro-2,3-dihydrobenzo[b][1,4]dioxine-5-carboxylate 2 (2.2 g, 9.20 mmol, 1 eq) was added to the mixture at 20° C. At 20° C. (inner temperature), Pd/C (660 mg, 10%) was added to the reaction mixture at 20° C. under H₂ atmosphere. After the addition, the suspension was degassed and purged with H₂ (15 psi) for 3 times, and then the mixture was stirred under H₂ (15 psi) atmosphere at 20° C. for 1 h. LC-MS showed starting material was consumed completely and desired mass was detected. The suspension was filtered through a pad of Celite and the filter cake was washed with THF (10 ml*3). The mixture was concentrated under reduced pressure to provide product (1.9 g, 9.08 mmol, 98% yield) as a white solid.

LCMS: RT=0.381. min, MS cal.: 209.1, [M+H]⁺=210.2

¹H NMR (400 MHz, CHLOROFORM-d) δ=6.76 (d, J=2.8 Hz, 1H), 6.42 (d, J=2.8 Hz, 1H), 4.31-4.24 (m, 4H), 3.87 (s, 3H), 3.55 (br s, 2H)

### Preparation of Intermediate Methyl 7-amino-6-bromo-2,3-dihydrobenzo[b][1,4]dioxine-5-carboxylate (4)

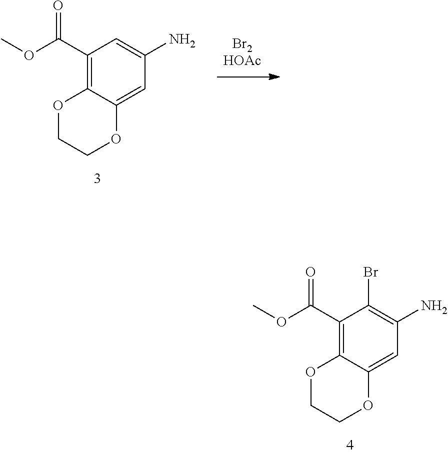 [SMILES: COC(=O)c1cc(N)cc2c1OCCO2 ; COC(=O)c1c(Br)c(N)cc2c1OCCO2]

To a solution of methyl 7-amino-2,3-dihydrobenzo[b][1,4]dioxine-5-carboxylate 3 (1.8 g, 8.60 mmol, 1 eq) in HOAc (13 mL) was added Br₂ (1.38 g, 8.60 mmol, 443 μL, 1 eq). The mixture was stirred at 0° C. for 1 h. LC-MS showed starting material was consumed completely and desired mass was detected. The mixture was poured into a mixture of ice water (50 mL) and extracted by DCM (25 ml*2). The organic phase was added with sat. NaHCO₃ adjust to pH=7˜8, and then the organic phase washed by brine (15 mL*2) dried over Na₂SO₄, filtered and concentrated. The residue was purified by column chromatography (SiO₂, Petroleum ether/Ethyl acetate=30/1 to 1/1) to provide product (1.9 g, 6.60 mmol, 76% yield) as a white solid.

LCMS: RT=0.603. min, MS cal.: 287.0, 289.0, [M+H]⁺=288.1

¹H NMR (400 MHz, DMSO-d6) δ=6.43 (s, 1H), 5.02 (s, 2H), 4.23-4.11 (m, 4H), 3.79 (s, 3H)

### Preparation of Intermediate Methyl 7-(2-acetoxyacetamido)-6-bromo-2,3-dihydrobenzo[b][1,4]dioxine-5-carboxylate (5)

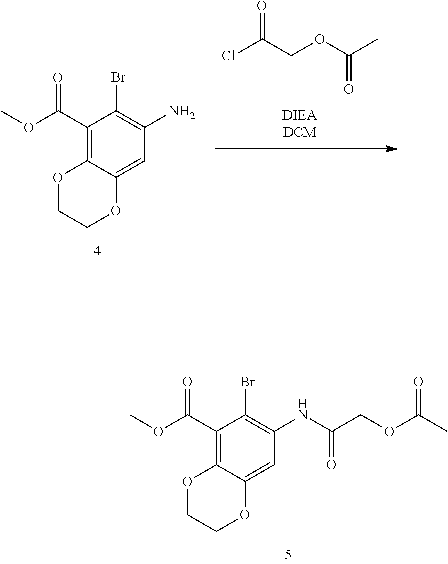

To a solution of methyl 7-amino-6-bromo-2,3-dihydrobenzo[b][1,4]dioxine-5-carboxylate 4 (2.4 g, 8.33 mmol, 1 eq) in DCM (24 mL) was added 2-chloro-2-oxoethyl acetate (1.14 g, 8.33 mmol, 896 μL, 1 eq) and DIEA (3.23 g, 25 mmol, 4.35 mL, 3 eq). The mixture was stirred at 0° C. for 2 h. LC-MS showed starting material was consumed completely and desired mass was detected. The mixture was diluted with H₂O (30 mL), and extracted by EtOAc (25 mL*2). Then organic phase was combined and washed by H₂O (30 mL), dried with Na₂SO₄, filtered. The organic phase was concentrated under reduced pressure at 40° C. to give a residue. The residue was purified by column chromatography (SiO₂, Petroleum ether/Ethyl acetate=30/1 to 0/1) to provide product (3.2 g, 8.24 mmol, 98% yield) as a white solid.

LCMS: RT=0.627. min, MS cal.: 387.0, 389.0, [M+H]⁺=388.1

¹H NMR (400 MHz, DMSO-d6) δ=9.60 (s, 1H), 7.13 (s, 1H), 4.66 (s, 2H), 4.30 (s, 4H), 3.84 (s, 3H), 2.12 (s, 3H)

### Preparation of Intermediate Methyl 7-(2-acetoxyacetamido)-6-bromo-8-nitro-2,3-dihydrobenzo[b][1,4]dioxine-5-carboxylate (6)

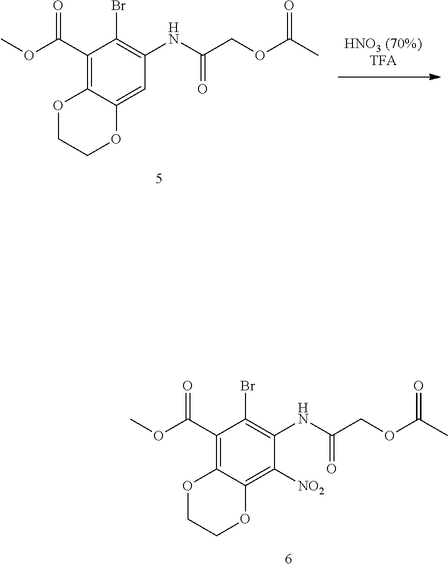

Equip a 100 mL three-necked round bottom flask and thermometer, N₂ balloon. TFA (22 mL) was charged to the three-necked round bottom flask, then methyl 7-(2-acetoxyacetamido)-6-bromo-2,3-dihydrobenzo[b][1,4]dioxine-5-carboxylate 5 (2.2 g, 5.67 mmol, 1 eq) was added to the mixture at 0° C. At 0° C. (inner temperature), HNO₃ (536 mg, 8.50 mmol, 383 μL, 70% purity, 1.5 eq) was added to the reaction mixture at 0° C. After the addition, the mixture was stirred at 0° C. for 2 h. LC-MS showed ˜30% of starting material remained. Then at 0° C. (inner temperature), HNO₃ (179 mg, 2.83 mmol, 128 μL, 70% purity, 0.5 eq) was added to the reaction mixture at 0° C. After the addition, the mixture was stirred at 0° C. for 2 h. LC-MS showed starting material was consumed completely and desired mass was detected. After 2 h, the reaction mixture was added to ice water (10 mL) at 0° C. The mixture was diluted with H₂O 10 mL, and extracted by EtOAc (25 mL*2). The organic phase was added with sat. NaHCO₃ adjust to pH=7˜8, and then washed by brine (20 mL*2) dried over Na₂SO₄, filtered and concentrated. The residue was purified by column chromatography (SiO₂, Petroleum ether/Ethyl acetate=20/1 to 0/1) to provide product (670 mg, 1.55 mmol, 27% yield) as a white solid.

LCMS: RT=0.636. min, MS cal.: 432.0, 434.0, [M+H]⁺=433.0

¹H NMR (400 MHz, DMSO-d6) δ=10.20 (s, 1H), 4.62 (s, 2H), 4.42-4.36 (m, 4H), 3.80 (s, 3H), 2.10 (s, 3H)

### Preparation of Intermediate Methyl 7-(2-acetoxyacetamido)-8-amino-2,3-dihydrobenzo[b][1,4]dioxine-5-carboxylate (7)

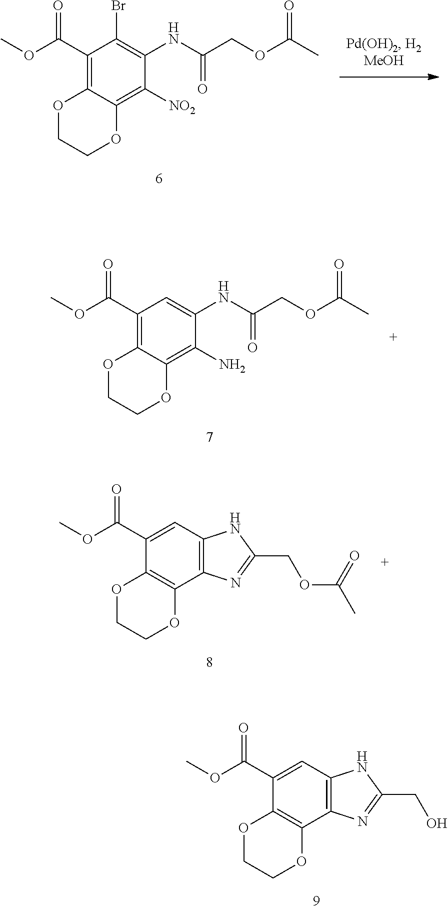

Equip a 35 mL Hydrogenated bottle, H₂ atmosphere. MeOH (6 mL) was charged to the round bottom flask, then methyl 7-(2-acetoxyacetamido)-6-bromo-8-nitro-2,3-dihydrobenzo[b][1,4]dioxine-5-carboxylate 6 (600 mg, 1.39 mmol, 1 eq) was added to the mixture at 20° C. At 20° C. (inner temperature), Pd(OH)₂ (180 mg, 1.28 mmol, 9.25e-1 eq) was added to the reaction mixture at 20° C. under H₂ atmosphere. After the addition, the suspension was degassed and purged with H₂ (50 psi) for 3 times, and then the mixture was stirred under H₂ (50 psi) atmosphere at 25° C. for 12 h. LC-MS showed starting material was consumed completely. The reaction mixture was diluted with MeOH (5 ml), and then filtered through a pad of Celite and the filter cake was washed with MeOH (10 mL*2). And the filtrate was concentrated under reduced pressure at 40° C. to give a mixture of 7/8/9 (400 mg in total) with a ratio of 2:5:1.

### Preparation of Intermediate Methyl 2-(acetoxymethyl)-7,8-dihydro-3H-[1,4]dioxino[2′,3′:3,4]benzo[1,2-d]imidazole-5-carboxylate (10)

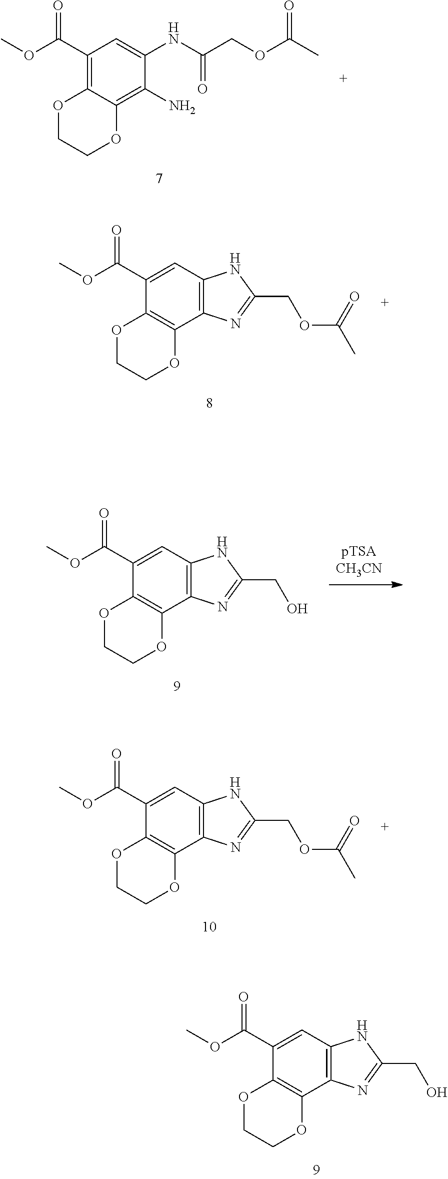

A mixture of 7/8/9 (350 mg in total) with a ratio of 2:5:1 in ACN (4 mL) was added 4-methylbenzenesulfonic acid (2.32 mg, 13.5 μmol, 0.1 eq). The mixture was stirred at 60° C. for 24 h. LC-MS showed 7/8/9 were consumed completely and desired mass was detected. The mixture was diluted with H₂O (20 mL), and extracted by EtOAc (20 mL*3). Then organic phase was combined and washed by brine (15 mL), dried with Na₂SO₄, filtered. The organic phase was concentrated under reduced pressure at 40° C. to give a residue. The crude product was purified by reversed-phase HPLC (column: Waters Xbridge Prep OBD C18 150*40 mm*10 um; mobile phase: [H₂O (10 mM NH₄HCO₃)-ACN]; gradient: 5%-45% B over 8.0 min) to provide a mixture of 9/10 (102 mg, crude) as a white solid.

LCMS: RT=0.494 min, MS cal.: 306.1, [M+H]⁺=307.2 Compound 8B (78 mg, crude) was obtained as a white solid.

LCMS: RT=0.428 min, MS cal.: 264.1, [M+H]⁺=264.2

### Preparation of Intermediate Methyl (S)-2-(acetoxymethyl)-3-(oxetan-2-ylmethyl)-7,8-dihydro-3H-[1,4]dioxino[2′,3′:3,4]benzo[1,2-d]imidazole-5-carboxylate (12) and Methyl (S)-2-(acetoxymethyl)-1-(oxetan-2-ylmethyl)-7,8-dihydro-1H-[1,4]dioxino[2′,3′:3,4]benzo[1,2-d]imidazole-5-carboxylate (13)

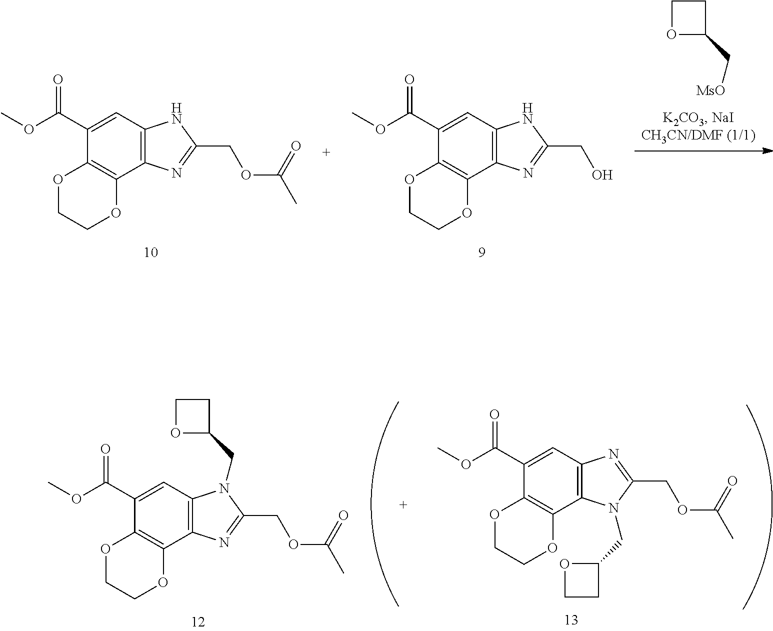

To a solution of 10/9 (100 mg, 327 μmol, 1 eq) in ACN (0.5 mL) and DMF (0.5 mL) was added K₂CO₃ (271 mg, 1.96 mmol, 6 eq), NaI (48.9 mg, 327 μmol, 1 eq) and(S)-oxetan-2-ylmethyl methanesulfonate (271 mg, 1.63 mmol, 5 eq). The mixture was stirred at 80° C. for 12 h. LC-MS showed starting material was consumed completely and desired mass was detected. The mixture was filtered and the filter cake was washed by ACN (5 ml*2). The reaction mixture was concentrated under reduced pressure to give a mixture of 12 and 13 (120 mg, 319 μmol, 97% yield in total) with a ratio of 1/1.

LCMS: RT=1.177. min, MS cal.: 376.1, [M+H]⁺=377.1

### Preparation of Intermediate Methyl (S)-2-(hydroxymethyl)-3-(oxetan-2-ylmethyl)-7,8-dihydro-3H-[1,4]dioxino[2′,3′:3,4]benzo[1,2-d]imidazole-5-carboxylate (14) and methyl (S)-2-(hydroxymethyl)-1-(oxetan-2-ylmethyl)-7,8-dihydro-1H-[1,4]dioxino[2′,3′:3,4]benzo[1,2-d]imidazole-5-carboxylate (15)

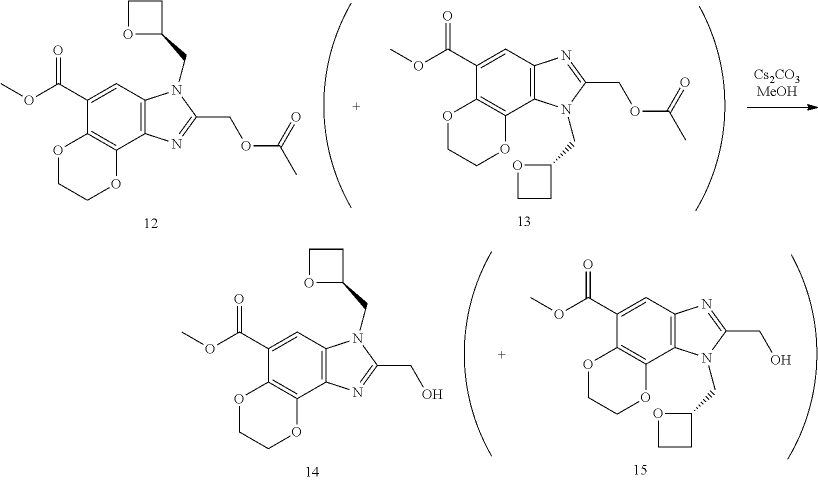

A mixture of methyl (S)-2-(hydroxymethyl)-3-(oxetan-2-ylmethyl)-7,8-dihydro-3H-[1,4]dioxino[2′,3′:3,4]benzo[1,2-d]imidazole-5-carboxylate (12) and methyl (S)-2-(hydroxymethyl)-1-(oxetan-2-ylmethyl)-7,8-dihydro-1H-[1,4]dioxino[2′,3′:3,4]benzo[1,2-d]imidazole-5-carboxylate (13) (120 mg, 319 μmol, 98% yield in total) with a ratio of 1/1 in MeOH (1.2 mL) was added Cs₂CO₃ (312 mg, 957 μmol, 3 eq). The mixture was stirred at 25° C. for 12 h. LC-MS showed starting materials was consumed completely and desired mass was detected. The mixture was concentrated under reduced pressure to give a residue. The crude product was purified by reversed-phase HPLC (column: Waters Xbridge Prep OBD C18 150*40 mm*10 um; mobile phase: [H₂O (10 mM NH₄HCO₃)-ACN]; gradient: 10%-30% B over 8.0 min) to provide compound 14 (20 mg, 59.8 μmol, 18% yield) as a white solid.

LCMS: RT=0.986 min, MS cal.: 334.1, [M+H]⁺=335.0

¹H NMR (400 MHz, DMSO-d6) δ=7.55 (s, 1H), 5.58 (br s, 1H), 5.01-4.99 (m, 1H), 4.78-4.66 (m, 2H), 4.65-4.56 (m, 1H), 4.52-4.42 (m, 2H), 4.39-4.26 (m, 5H), 3.79 (s, 3H), 2.69-2.62 (m, 1H), 2.40-2.30 (m, 1H)

And compound 15 (30 mg, 89.7 μmol, 28% yield) as a white solid.

LCMS: RT=1.017 min, MS cal.: 334.1, [M+H]⁺=335.0

¹H NMR (400 MHz, DMSO-d6) δ=7.52 (s, 1H), 5.58 (br s, 1H), 5.04-5.02 (m, 1H), 4.79-4.66 (m, 3H), 4.60-4.58 (m, 1H), 4.51-4.44 (m, 1H), 4.44-4.29 (m, 5H), 3.78 (s, 3H), 2.71-2.61 (m, 1H), 2.46-2.37 (m, 1H)

### Preparation of Preparation of Intermediate Methyl (S)-2-(((methylsulfonyl)oxy)methyl)-3-(oxetan-2-ylmethyl)-7,8-dihydro-3H-[1,4]dioxino[2′,3′:3,4]benzo[1,2-d]imidazole-5-carboxylate (16)

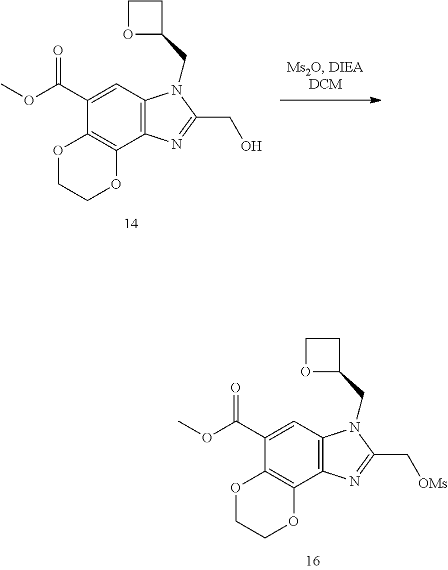 [SMILES: COC(=O)c1cc2c(nc(CO)n2C[C@@H]2CCO2)c2c1OCCO2 ; COC(=O)c1cc2c(nc(COS(C)(=O)=O)n2C[C@@H]2CCO2)c2c1OCCO2]

To a solution of methyl (S)-2-(hydroxymethyl)-3-(oxetan-2-ylmethyl)-7,8-dihydro-3H-[1,4]dioxino[2′,3′:3,4]benzo[1,2-d]imidazole-5-carboxylate 14 (30 mg, 89.7 μmol, 1 eq) in DCM (0.3 mL) was added Ms₂O (23.5 mg, 135 μmol, 1.5 eq) and DIEA (34.8 mg, 269 μmol, 46.9 μL, 3 eq). The mixture was stirred at 25° C. for 2 h. LC-MS showed starting material was consumed completely and desired mass was detected. The mixture was diluted with H₂O (5 mL), and extracted by DCM (5 mL*2). Then organic phase was combined and washed by H₂O (5 mL*2), dried with Na₂SO₄, filtered. The organic phase was concentrated under reduced pressure at 40° C. to provide product (37 mg, 89.7 μmol, 99% yield) as a white solid.

LCMS: RT=0.555 min, MS cal.: 412.1, [M+H]⁺=413.1

### Preparation of Intermediate Methyl 2-((4-((S)-2-(5-chloropyridin-2-yl)-2-methylbenzo[d][1,3]dioxol-4-yl)piperidin-1-yl)methyl)-3-(((S)-oxetan-2-yl)methyl)-7,8-dihydro-3H-[1,4]dioxino[2′,3′:3,4]benzo[1,2-d]imidazole-5-carboxylate (17)

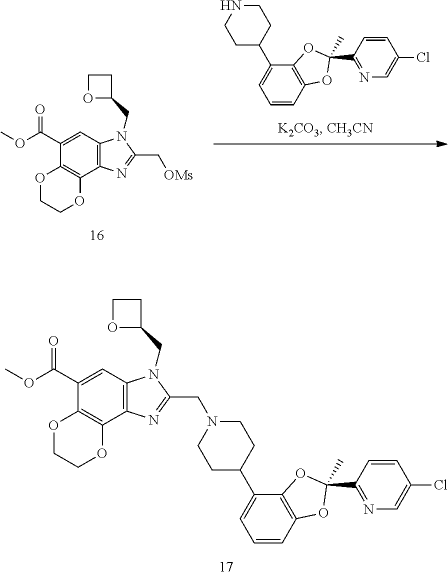 [SMILES: COC(=O)c1cc2c(nc(COS(C)(=O)=O)n2C[C@@H]2CCO2)c2c1OCCO2 ; C[C@]1(c2ccc(Cl)cn2)Oc2cccc(C3CCNCC3)c2O1 ; COC(=O)c1cc2c(nc(CN3CCC(c4cccc5c4O[C@@](C)(c4ccc(Cl)cn4)O5)CC3)n2C[C@@H]2CCO2)c2c1OCCO2]

To a solution of methyl (S)-2-(((methylsulfonyl)oxy)methyl)-3-(oxetan-2-ylmethyl)-7,8-dihydro-3H-[1,4]dioxino[2′,3′:3,4]benzo[1,2-d]imidazole-5-carboxylate 16 (37 mg, 89.7 μmol, 1 eq) in ACN (0.5 mL) was added K₂CO₃ (37.2 mg, 269 μmol, 3 eq) and(S)-5-chloro-2-(2-methyl-4-(piperidin-4-yl)benzo[d][1,3]dioxol-2-yl)pyridine (32.7 mg, 98.7 μmol, 1.1 eq). The mixture was stirred at 60° C. for 4 h. LC-MS showed starting material was consumed completely and desired mass was detected. The mixture was concentrated under reduced pressure to give a residue. The crude product was purified by reversed-phase HPLC (column: Waters Xbridge BEH C18 100*30 mm*10 um; mobile phase: [H₂O (10 mM NH₄HCO₃)-ACN]; gradient: 40%-90% B over 8.0 min) to provide product (18 mg, 27.8 μmol, 31% yield) as a white solid.

LCMS: RT=2.192 min, MS cal.: 646.2, 647.2, [M+H]⁺=647.1

¹H NMR (400 MHz, DMSO-d6) δ=8.71 (d, J=2.4 Hz, 1H), 8.00 (dd, J=2.4, 8.4 Hz, 1H), 7.60 (d, J=8.4 Hz, 1H), 7.55 (s, 1H), 6.82-6.72 (m, 3H), 5.05-5.03 (m, 1H), 4.70-4.61 (m, 1H), 4.57-4.49 (m, 1H), 4.48-4.40 (m, 1H), 4.39-4.26 (m, 5H), 3.87 (d, J=13.2 Hz, 1H), 3.79 (s, 3H), 3.71 (d, J=13.6 Hz, 1H), 2.98-2.96 (m, 1H), 2.83-2.81 (m, 1H), 2.68-2.60 (m, 2H), 2.42-2.31 (m, 1H), 2.26-2.09 (m, 2H), 2.00 (s, 3H), 1.79-1.65 (m, 4H)

### Preparation of 2-((4-((S)-2-(5-Chloropyridin-2-yl)-2-methylbenzo[d][1,3]dioxol-4-yl)piperidin-1-yl)methyl)-3-(((S)-oxetan-2-yl)methyl)-7,8-dihydro-3H-[1,4]dioxino[2′,3′:3,4]benzo[1,2-d]imidazole-5-carboxylic acid Compound 93 (18)

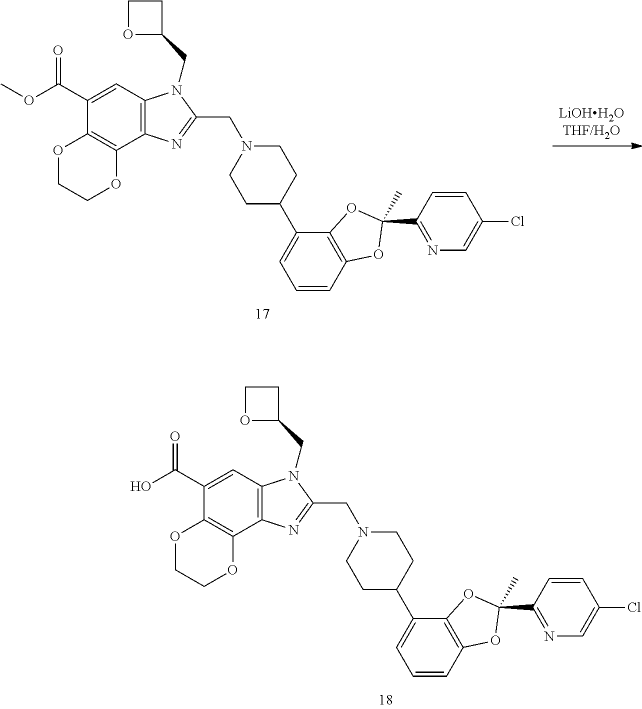 [SMILES: COC(=O)c1cc2c(nc(CN3CCC(c4cccc5c4O[C@@](C)(c4ccc(Cl)cn4)O5)CC3)n2C[C@@H]2CCO2)c2c1OCCO2 ; C[C@]1(c2ccc(Cl)cn2)Oc2cccc(C3CCN(Cc4nc5c6c(c(C(=O)O)cc5n4C[C@@H]4CCO4)OCCO6)CC3)c2O1]

To a solution of methyl 2-((4-((S)-2-(5-chloropyridin-2-yl)-2-methylbenzo[d][1,3]dioxol-4-yl)piperidin-1-yl)methyl)-3-(((S)-oxetan-2-yl)methyl)-7,8-dihydro-3H-[1,4]dioxino[2′,3′:3,4]benzo[1,2-d]imidazole-5-carboxylate 17 (18 mg, 27.8 μmol, 9.82e-1 eq) in THF (0.35 mL) and H₂O (0.15 mL) was added LiOH·H₂O (1.55 mg, 36.8 μmol, 1.3 eq). The mixture was stirred at 25° C. for 12 h. LC-MS showed starting material was consumed completely and desired mass was detected. The mixture was concentrated under reduced pressure to give a residue. The crude product was purified by reversed-phase HPLC (column: Waters Xbridge Prep OBD C18 150*40 mm*10 um; mobile phase: [H₂O (10 mM NH₄HCO₃)-ACN]; gradient: 20%-60% B over 8.0 min) to provide product (14.6 mg, 22.7 μmol, 80% yield, 98% purity) as a white solid.

LCMS: RT=2.533 min, MS cal.: 632.2, 633.2, [M+H]⁺=633.2

HPLC: RT=10.068 min, purity: 98%

¹H NMR (400 MHz, DMSO-d₆) δ=8.71 (d, J=2.0 Hz, 1H), 8.00 (dd, J=2.4, 8.4 Hz, 1H), 7.60 (d, J=8.4 Hz, 1H), 7.33 (s, 1H), 6.83-6.72 (m, 3H), 5.08-5.01 (m, 1H), 4.51-4.50 (m, 1H), 4.68-4.43 (m, 2H), 4.36-4.29 (m, 3H), 4.25-4.23 (m, 2H), 3.84 (d, J=13.2 Hz, 1H), 3.70 (d, J=13.2 Hz, 1H), 2.97-2.95 (m, 1H), 2.84-2.82 (m, 1H), 2.64-6.62 (m, 1H), 2.38-2.36 (m, 1H), 2.25-2.08 (m, 3H), 2.01 (s, 3H), 1.79-1.65 (m, 4H)

### Example A35. —Preparation of Compound 94. 2-((R)-1-(4-(6-((4-Cyano-2-fluorobenzyl)oxy)pyridin-2-yl)piperidin-1-yl)ethyl)-4-methoxy-1-(((S)-oxetan-2-yl)methyl)-1H-benzo[d]imidazole-6-carboxylic acid (Compound 94) (2)

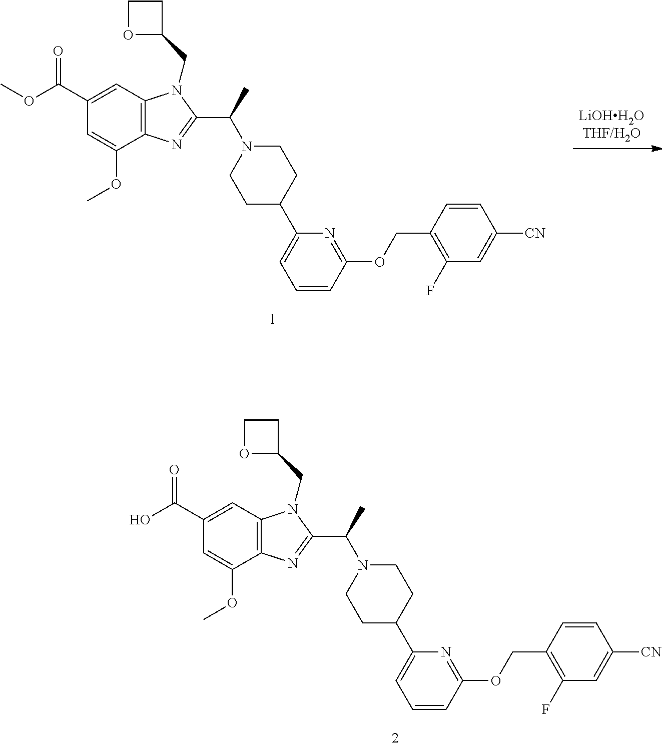 [SMILES: COC(=O)c1cc(OC)c2nc([C@@H](C)N3CCC(c4cccc(OCc5ccc(C#N)cc5F)n4)CC3)n(C[C@@H]3CCO3)c2c1 ; COc1cc(C(=O)O)cc2c1nc([C@@H](C)N1CCC(c3cccc(OCc4ccc(C#N)cc4F)n3)CC1)n2C[C@@H]1CCO1]

To a solution of methyl 2-((R)-1-(4-(6-((4-cyano-2-fluorobenzyl)oxy)pyridin-2-yl)piperidin-1-yl)ethyl)-4-methoxy-1-(((S)-oxetan-2-yl)methyl)-1H-benzo[d]imidazole-6-carboxylate 1 (80 mg, 130 μmol, 1 eq) in THF (0.7 mL) and H₂O (0.3 mL) was added LiOH·H₂O (5.47 mg, 130 μmol, 1 eq). The mixture was stirred at 25° C. for 12 h. LCMS showed ˜24% of starting material remained. Several new peaks were shown on LCMS and ˜72% of desired compound was detected. The reaction mixture was filtered and concentrated under reduced pressure to give a residue in ACN. The residue was purified by prep-HPLC (column: Waters Xbridge Prep OBD C18 150*40 mm*10 um; mobile phase: [H₂O (10 mM NH₄HCO₃)-ACN]; gradient: 20%-70% B over 8.0 min) to provide product (25.6 mg, 42.8 μmol, 33% yield) as a white solid.

LCMS: RT=0.410 min, MS cal.: 599.3, [M+H]⁺=600.2

LCMS: RT=2.576 min, MS cal.: 599.3, [M+H]⁺=600.3

HPLC: RT=10.232 min, purity: 100%

¹H NMR (400 MHz, METHANOL-d4) δ=7.87 (d, J=0.8 Hz, 1H), 7.68-7.63 (m, 1H), 7.61-7.56 (m, 1H), 7.56-7.49 (m, 2H), 7.47 (d, J=0.8 Hz, 1H), 6.86-6.79 (d, J=7.2 Hz, 1H), 6.70-6.65 (d, J=8.4 Hz, 1H), 5.50 (s, 2H), 5.34-5.25 (m, 1H), 4.71-4.53 (m, 4H), 4.32-4.23 (m, 1H), 4.06 (s, 3H), 3.17-3.03 (m, 2H), 2.86-2.76 (m, 1H), 2.76-2.69 (m, 1H), 2.69-2.58 (m, 1H), 2.57-2.48 (m, 1H), 2.47-2.36 (m, 1H), 1.95-1.69 (m, 4H), 1.65 (d, J=6.8 Hz, 3H)

### Example A36. Preparation of Compound 95. 2-((S)-1-(4-(6-((4-Cyano-2-fluorobenzyl)oxy)pyridin-2-yl)piperidin-1-yl)ethyl)-4-methoxy-1-(((S)-oxetan-2-yl)methyl)-1H-benzo[d]imidazole-6-carboxylic acid (Compound 95) (9)

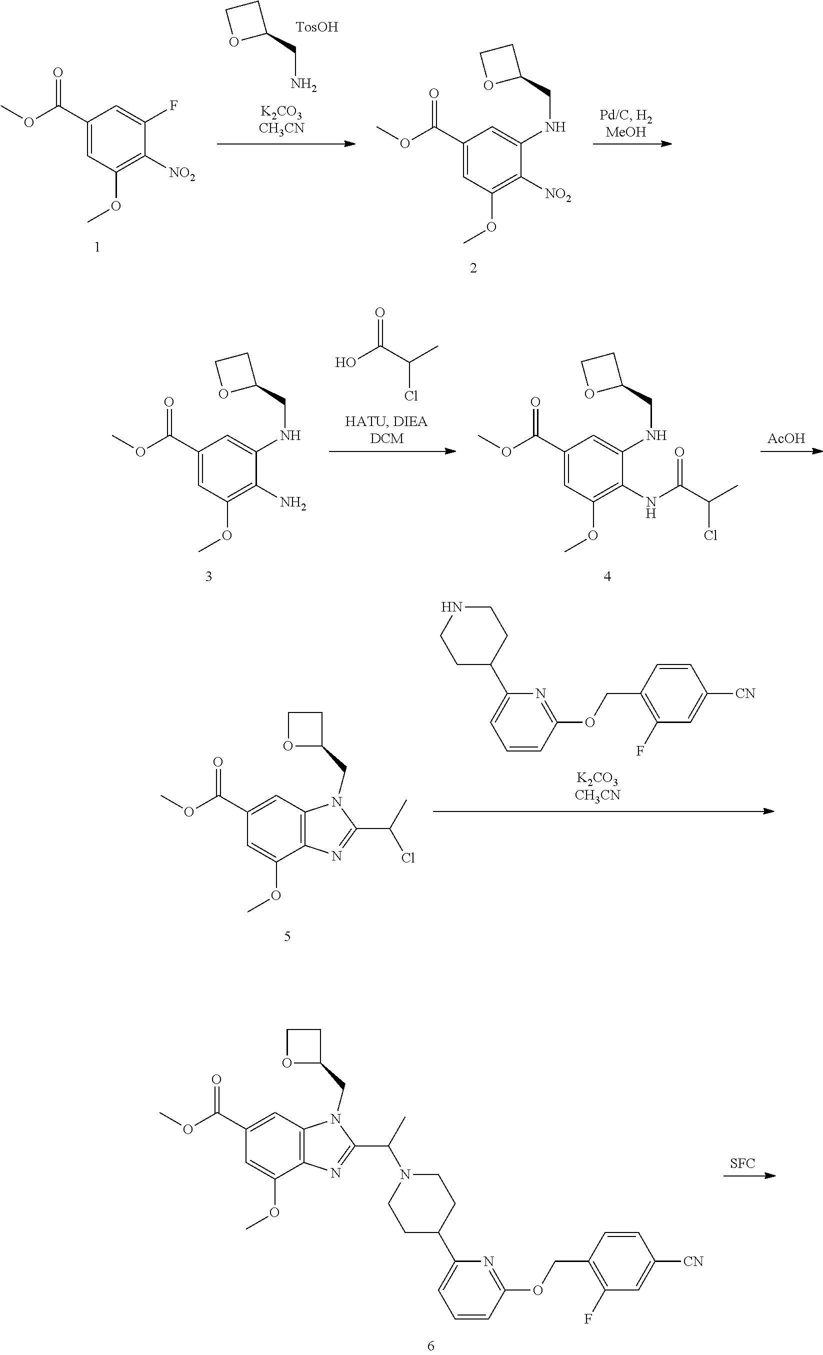

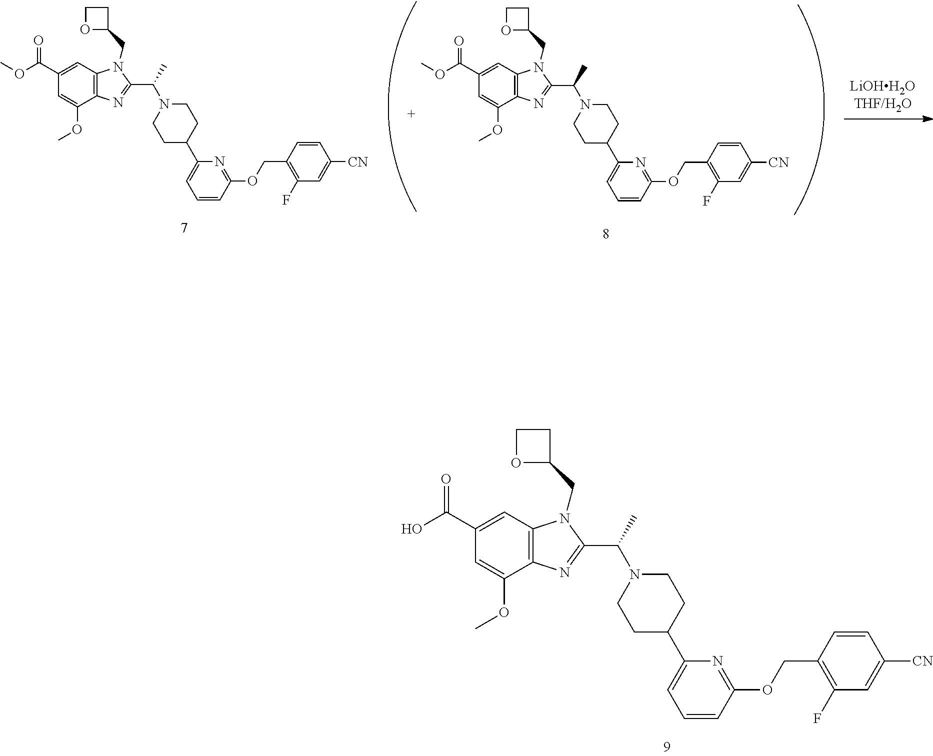

### Preparation of Intermediate Methyl (S)-3-methoxy-4-nitro-5-((oxetan-2-ylmethyl)amino)benzoate (2)

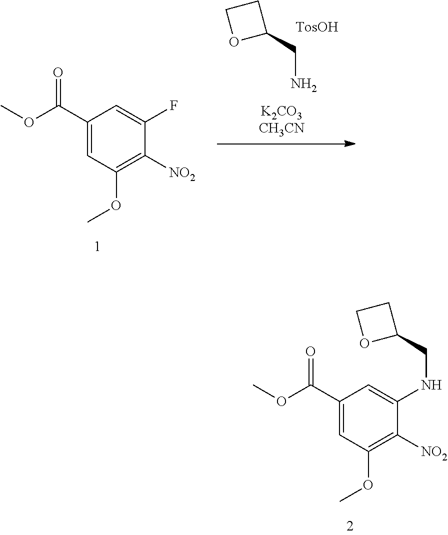

To a solution of methyl 3-fluoro-5-methoxy-4-nitrobenzoate 1 (0.8 g, 3.49 mmol, 1 eq) and (S)-oxetan-2-ylmethanamine (1.36 g, 5.24 mmol, 1.5 eq, p-TSA) in CH₃CN (10 mL) was added K₂CO₃ (1.45 g, 10.5 mmol, 3 eq). The mixture was stirred at 60° C. for 4 h. TLC (Petroleum ether/Ethyl acetate=5:1) indicated starting material was consumed completely and one new spot formed. The reaction was clean according to TLC. LCMS showed starting material was consumed completely and one main peak with desired was detected. The reaction mixture was partitioned between ethyl acetate (30 mL) and H₂O (15 mL). The organic phase was separated, washed with brine (5 mL), dried over Na₂SO₄, filtered, and concentrated under reduced pressure to give a residue. The residue was purified by column chromatography (SiO₂, Petroleum ether/Ethyl acetate=100/0 to 87/13) to provide product (1 g, 3.38 mmol, 97% yield) as a white solid.

LCMS: RT=1.713 min, MS cal.: 296.1, [M+H]⁺=297.1

¹H NMR (400 MHz, CHLOROFORM-d) δ=7.14 (d, J=1.2 Hz, 1H), 6.97 (d, J=1.2 Hz, 1H), 5.12-5.05 (m, 1H), 4.75-4.69 (m, 1H), 4.60-4.56 (m, 1H), 3.94 (s, 6H), 3.49 (d, J=4.4 Hz, 2H), 2.77-2.68 (m, 1H), 2.58-2.56 (m, 1H)

### Preparation of Intermediate Methyl (S)-4-amino-3-methoxy-5-((oxetan-2-ylmethyl)amino)benzoate (3)

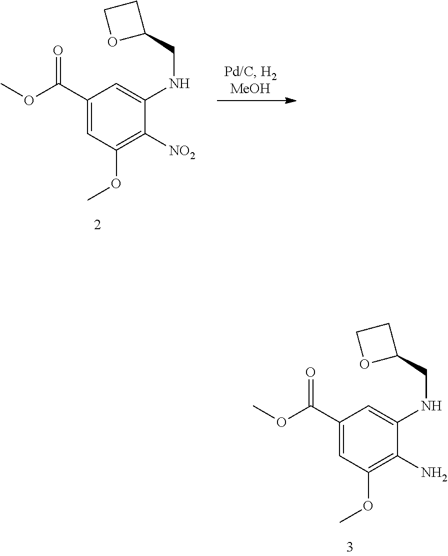 [SMILES: COC(=O)c1cc(NC[C@@H]2CCO2)c([N+](=O)[O-])c(OC)c1 ; "Pd/C, H2MeOH" ; COC(=O)c1cc(NC[C@@H]2CCO2)c(N)c(OC)c1]

Equip a 25 mL round bottom flask, H₂ balloon (15 psi). THF (2 mL) was charged to the 25 mL round bottom flask under N₂ atmosphere, then Pd/C (8.00 g, 10%) was added at 20° C. within 1 min. At 20° C., methyl (S)-3-methoxy-4-nitro-5-((oxetan-2-ylmethyl)amino)benzoate 2 (0.8 g, 2.70 mmol, 1 eq) in THF (2 mL) was added to the reaction mixture at 20° C. within 1 min, purged with H₂ (15 psi) for 3 times. After the addition, the mixture was stirred at 20° C. for 1 h under H₂ (15 psi). The reaction was monitored by TLC and LCMS showed starting material was consumed completely and one main peak with desired was detected. After 1 h, the reaction mixture was filtered and concentrated under reduced pressure to provide product (1 g, crude) as a white solid.

LCMS: RT=0.330 min, MS cal.: 266.1, [M+H]⁺=267.0

¹H NMR (400 MHz, DMSO-d₆) δ=7.18 (s, 1H), 7.16 (s, 1H), 5.14-5.05 (m, 1H), 4.79-4.69 (m, 1H), 4.62-4.62 (m, 1H), 3.89 (s, 3H), 3.87 (s, 3H), 3.78 (br s, 2H), 3.49-3.33 (m, 2H), 2.79-2.69 (m, 1H), 2.64-2.53 (m, 1H)

### Preparation of Intermediate Methyl 4-(2-chloropropanamido)-3-methoxy-5-((((S)-oxetan-2-yl)methyl) amino)benzoate (4)

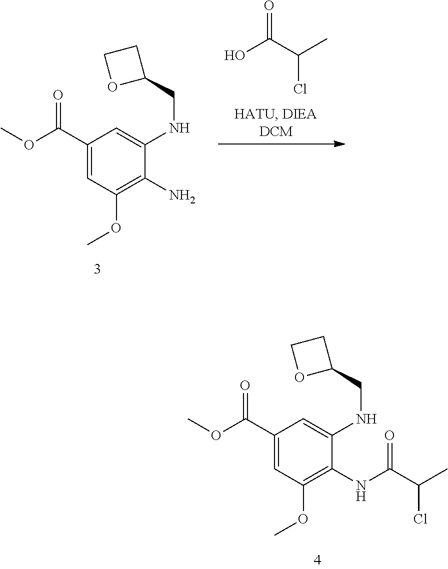

To a solution of methyl (S)-4-amino-3-methoxy-5-((oxetan-2-ylmethyl)amino)benzoate 3 (700 mg, 2.63 mmol, 1 eq) and 2-chloropropanoic acid (342 mg, 3.15 mmol, 1.2 eq) in DCM (7.5 mL) was added HATU (1.10 g, 2.89 mmol, 1.1 eq) and DIEA (1.70 g, 13.1 mmol, 2.29 mL, 5 eq). The mixture was stirred at 50° C. for 1 h. LC-MS showed starting material was consumed completely and one main peak with desired was detected. TLC (Petroleum ether/Ethyl acetate=5:1) indicated starting material was consumed completely and one new spot formed. The reaction was clean according to TLC. The reaction mixture was partitioned between ethyl acetate (20 mL) and H₂O (15 mL). The organic phase was separated, washed with brine (3 mL), dried over Na₂SO₄, filtered, and concentrated under reduced pressure to give a residue. The residue was purified by column chromatography (SiO₂, Petroleum ether/Ethyl acetate=100/0 to 90/10) to provide product (820 mg, 2.30 mmol, 87% yield) as a white solid.

LCMS: RT=0.400 min, MS cal.: 356.1, 358.1, [M+H]⁺=357.1

### Preparation of Intermediate Methyl 2-(1-chloroethyl)-4-methoxy-1-(((S)-oxetan-2-yl)methyl)-1H-benzo[d]imidazole-6-carboxylate (5)

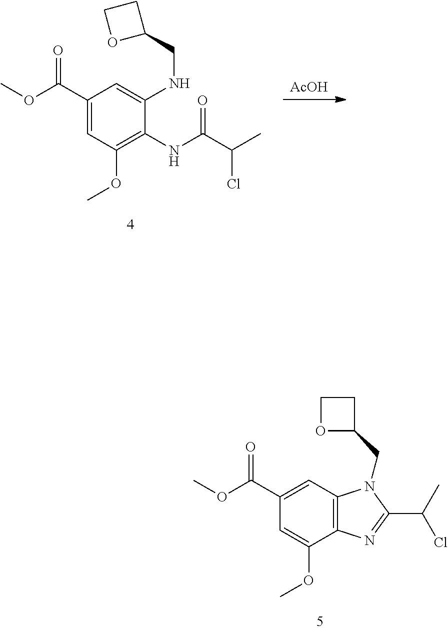 [SMILES: "AcOH" ; COC(=O)c1cc(NC[C@@H]2CCO2)c(NC(=O)C(C)Cl)c(OC)c1 ; COC(=O)c1cc(OC)c2nc(C(C)Cl)n(C[C@@H]3CCO3)c2c1]

A solution of methyl 4-(2-chloropropanamido)-3-methoxy-5-((((S)-oxetan-2-yl)methyl) amino)benzoate 4 (770 mg, 2.16 mmol, 1 eq) in AcOH (8 mL) was stirred at 100° C. for 1 h. LC-MS showed starting material was consumed completely and one main peak with desired was detected. TLC (Petroleum ether/Ethyl acetate=0:1) indicated starting material was consumed completely and one new spot formed. The reaction was clean according to TLC. The reaction mixture was partitioned between ethyl acetate (30 mL) and H₂O (15 mL). The organic phase was separated, washed with brine (3 mL), dried over Na₂SO₄, filtered and concentrated under reduced pressure to give a residue. The residue was purified by column chromatography (SiO₂, Petroleum ether/Ethyl acetate=100/0 to 79/21) to provide product (320 mg, 945 μmol, 44% yield) as a white solid.

LCMS: RT=0.411 min, MS cal.: 338.1, 340.1, [M+H]⁺=339.0

### Preparation of Intermediate Methyl 2-(1-(4-(6-((4-cyano-2-fluorobenzyl)oxy)pyridin-2-yl)piperidin-1-yl)ethyl)-4-methoxy-1-(((S)-oxetan-2-yl)methyl)-1H-benzo[d]imidazole-6-carboxylate (6)

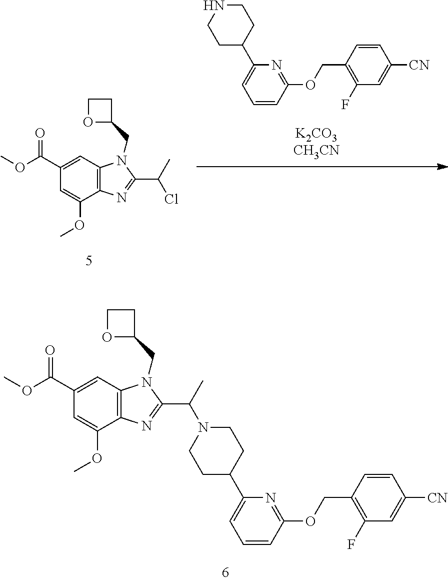 [SMILES: COC(=O)c1cc(OC)c2nc(C(C)Cl)n(C[C@@H]3CCO3)c2c1 ; N#Cc1ccc(COc2cccc(C3CCNCC3)n2)c(F)c1 ; COC(=O)c1cc(OC)c2nc(C(C)N3CCC(c4cccc(OCc5ccc(C#N)cc5F)n4)CC3)n(C[C@@H]3CCO3)c2c1]

To a solution of methyl 2-(1-chloroethyl)-4-methoxy-1-(((S)-oxetan-2-yl)methyl)-1H-benzo[d]imidazole-6-carboxylate 5 (290 mg, 856 μmol, 1 eq) and 3-fluoro-4-(((6-(piperidin-4-yl)pyridin-2-yl)oxy)methyl)benzonitrile (266.52 mg, 856.00 μmol, 1 eq) in CH₃CN (3 mL) was added K₂CO₃ (355 mg, 2.57 mmol, 3 eq). The mixture was stirred at 60° C. for 4 h. LC-MS showed starting material was consumed completely and one main peak with desired was detected. TLC (Petroleum ether/Ethyl acetate=0:1) indicated starting material was consumed completely and one new spot formed. The reaction was clean according to TLC. The reaction mixture was partitioned between ethyl acetate (20 mL) and H₂O (10 mL). The organic phase was separated, washed with brine (3 mL), dried over Na₂SO₄, filtered, and concentrated under reduced pressure to give a residue. The residue was purified by column chromatography (SiO₂, Petroleum ether/Ethyl acetate=100/0 to 75/25). And crude product was purified by prep-HPLC (column: Waters Xbridge BEH C18 100*30 mm*10 um; mobile phase: [H₂O (10 mM NH₄HCO₃)-ACN]; gradient: 60%-95% B over 8.0 min) to provide product (150 mg, 244 μmol, 29% yield) as a white solid.

LCMS: RT=2.469 min, MS cal.: 613.3, 614.3, [M+H]⁺=614.2

### Preparation of Intermediate Methyl 2-((S)-1-(4-(6-((4-cyano-2-fluorobenzyl)oxy)pyridin-2-yl)piperidin-1-yl)ethyl)-4-methoxy-1-(((S)-oxetan-2-yl)methyl)-1H-benzo[d]imidazole-6-carboxylate (7) and Methyl 2-((R)-1-(4-(6-((4-cyano-2-fluorobenzyl)oxy)pyridin-2-yl)piperidin-1-yl)ethyl)-4-methoxy-1-(((S)-oxetan-2-yl)methyl)-1H-benzo[d]imidazole-6-carboxylate (8)

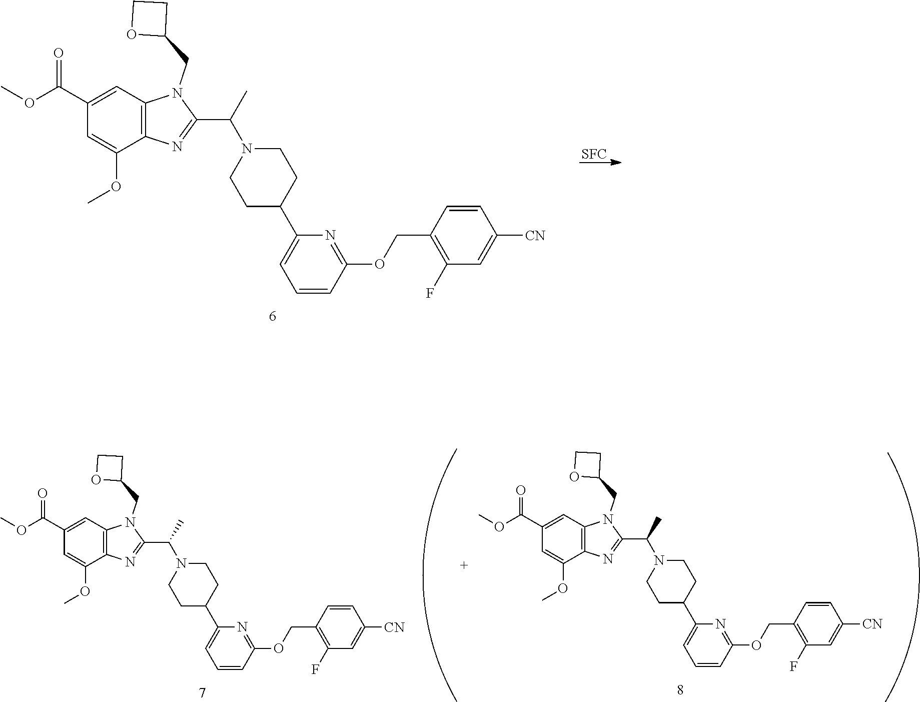

The methyl 2-(1-(4-(6-((4-cyano-2-fluorobenzyl)oxy)pyridin-2-yl)piperidin-1-yl)ethyl)-4-methoxy-1-(((S)-oxetan-2-yl)methyl)-1H-benzo[d]imidazole-6-carboxylate 6 (150 mg) was further separated by SFC (column: DAICEL CHIRALCEL OD (250 mm*30 mm, 10 um); mobile phase: [CO₂-IPA (0.1% NH₃H₂O)]; B %: 50%, isocratic elution mode) to provide 7 (60 mg, 97.8 μmol, 30% yield) and 8 (90 mg, 147 μmol, 45% yield) as a white solids.

LCMS Compound 7: RT=0.445 min, MS cal.: 613.3, 614.3, [M+H]⁺=614.3.

HPLC Compound 7: RT=3.086 min, purity: 96.18%.

SFC Compound 7: RT=1.320 min, purity: 100%

LCMS Compound 8: RT=0.468 min, MS cal.: 613.3, 614.3, [M+H]⁺=614.3.

HPLC Compound 8: RT=3.038 min, purity: 99.07%

SFC Compound 8: RT=1.320 min, purity: 100%

### Preparation of 2-((S)-1-(4-(6-((4-Cyano-2-fluorobenzyl)oxy)pyridin-2-yl)piperidin-1-yl)ethyl)-4-methoxy-1-(((S)-oxetan-2-yl)methyl)-1H-benzo[d]imidazole-6-carboxylic acid Compound 95 (9)

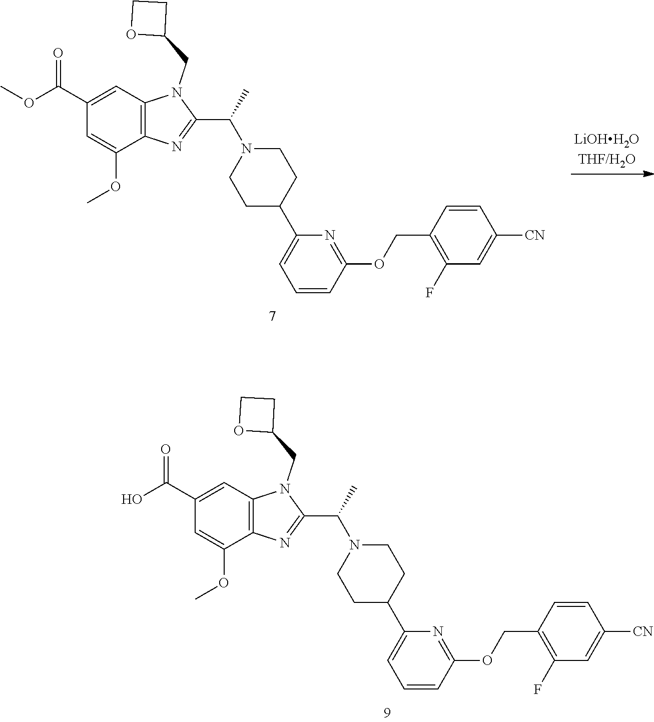 [SMILES: COC(=O)c1cc(OC)c2nc([C@H](C)N3CCC(c4cccc(OCc5ccc(C#N)cc5F)n4)CC3)n(C[C@@H]3CCO3)c2c1 ; COc1cc(C(=O)O)cc2c1nc([C@H](C)N1CCC(c3cccc(OCc4ccc(C#N)cc4F)n3)CC1)n2C[C@@H]1CCO1]

To a solution of methyl 2-((S)-1-(4-(6-((4-cyano-2-fluorobenzyl)oxy)pyridin-2-yl)piperidin-1-yl)ethyl)-4-methoxy-1-(((S)-oxetan-2-yl)methyl)-1H-benzo[d]imidazole-6-carboxylate 7 (50 mg, 81.5 μmol, 1 eq) in THF (0.7 mL) and H₂O (0.3 mL) was added LiOH·H₂O (1.71 mg, 40.7 μmol, 0.5 eq). The mixture was stirred at 20° C. for 12 h. LC-MS showed starting material was consumed completely and one main peak with desired was detected. The reaction mixture was filtered and concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 25%-55% B over 8.0 min) to provide product (13.8 mg, 22.5 μmol, 28% yield, 98% purity) as a white solid.

LCMS: RT=2.567 min, MS cal.: 599.3, 600.3, [M+H]⁺=600.3

HPLC: RT=10.189 min, purity=98.59%

¹H NMR (400 MHz, METHANOL-d₄) δ=7.90-7.84 (m, 2H), 7.73-7.67 (m, 2H), 7.64 (t, J=8.0 Hz, 1H), 7.25 (s, 1H), 6.87 (d, J=7.2 Hz, 1H), 6.71 (d, J=8.0 Hz, 1H), 5.47 (s, 2H), 5.06-5.04 (m, 1H), 4.93-4.82 (m, 1H), 4.54-4.46 (m, 3H), 4.42 (q, J=7.2 Hz, 1H), 3.94 (s, 3H), 3.06-3.04 (m, 1H), 2.75-2.72 (m, 1H), 2.69-2.60 (m, 1H), 2.57-2.54 (m, 1H), 2.26-2.24 (m, 1H), 1.89-1.81 (m, 1H), 1.80-1.66 (m, 2H), 1.57-1.47 (m, 1H), 1.42 (d, J=6.8 Hz, 3H)

### Example A37. —Preparation of Compound 96. 2-((S)-1-(4-((S)-2-(5-Chloropyridin-2-yl)-2-methylbenzo[d][1,3]dioxol-4-yl)piperidin-1-yl)ethyl)-4-methoxy-1-methyl-1H-benzo[d]imidazole-6-carboxylic acid (Compound 96) (9)

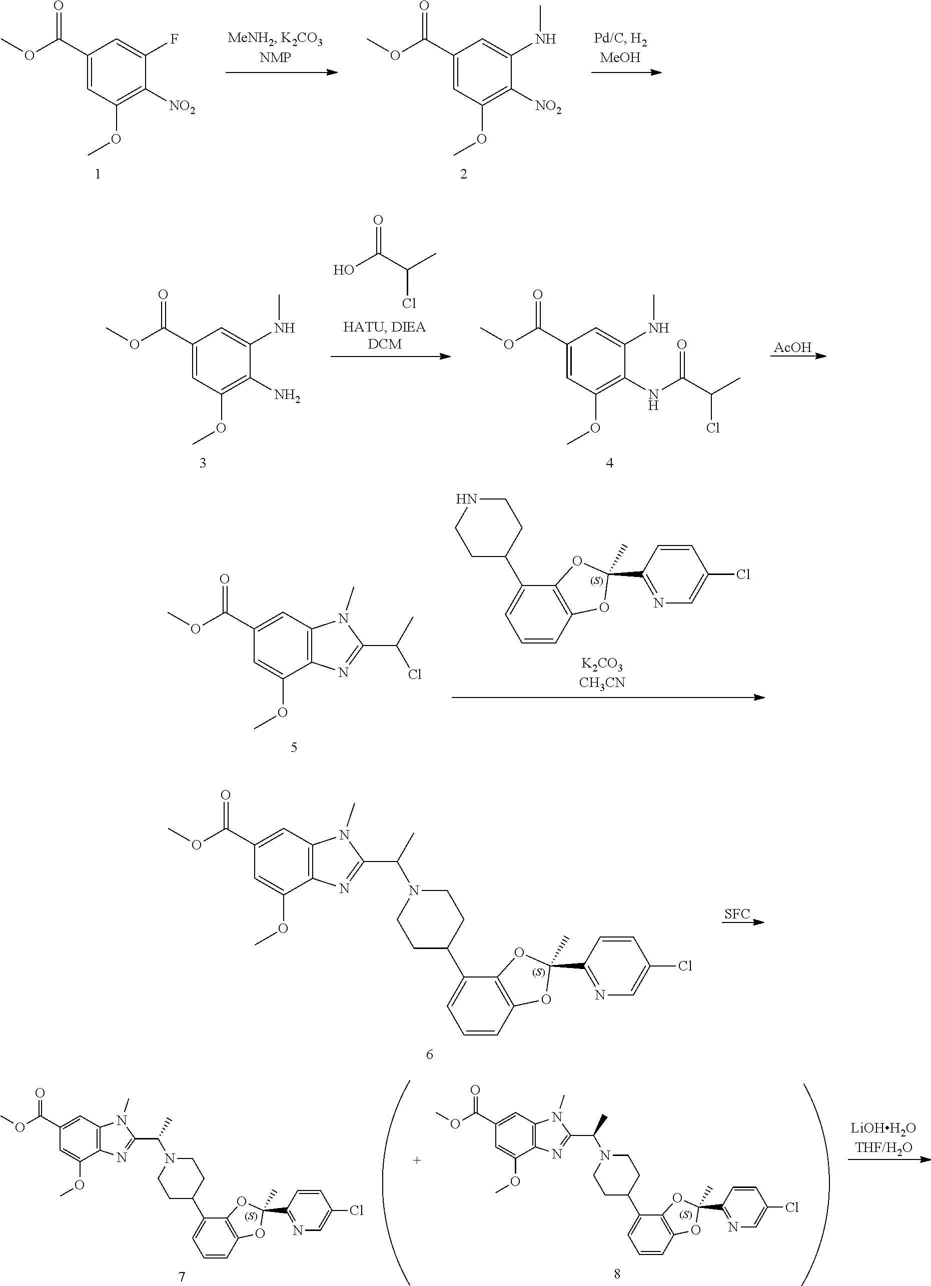

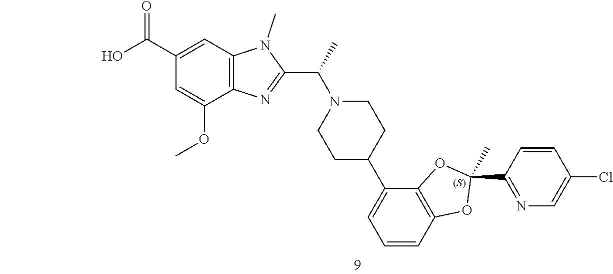

### Preparation of Intermediate Methyl 3-methoxy-5-(methylamino)-4-nitrobenzoate (2)

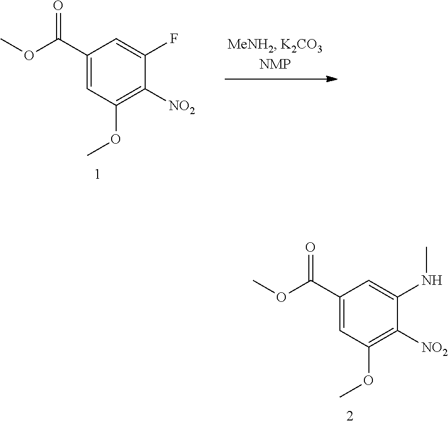 [SMILES: COC(=O)c1cc(F)c([N+](=O)[O-])c(OC)c1 ; CNc1cc(C(=O)OC)cc(OC)c1[N+](=O)[O-]]

Equip a 25 mL round bottom flask, and N₂ balloon. NMP (5 mL) was charged to the 25 mL three-necked round bottom flask, methyl 3-fluoro-5-methoxy-4-nitrobenzoate 1 (500 mg, 2.18 mmol, 1 eq) and methylamine (2 M, 2.18 mL, 2 eq) was added at 25° C. within 2 min. At 25° C. (inner temperature), K₂CO₃ (1.51 g, 10.9 mmol, 5 eq) was added to the mixture at 25° C. within 2 min. After the addition, the mixture was stirred at 25° C. for 8 hr. HPLC showed the starting material was consumed completely. After 2 h, the mixture was added FA (1 M) to pH=5 at 25° C. The mixture was extracted by EtOAc 300 mL (100 mL*3). Then organic phase was combined and washed by H₂O 100 mL, brine 100 mL, dried with Na₂SO₄, filtered. The organic phase was concentrated under reduced pressure at 45° C. to give a residue. The residue was purified by column chromatography (SiO₂, PE/EtOAc=10/1 to 0/1) to provide product (500 mg, 2.08 mmol, 95% yield) as an orange solid.

HPLC: product: RT=1.968 min; start material: RT=2.159 min, purity: 95.08%

¹H NMR (400 MHz, CHLOROFORM-d) δ=7.08 (d, J=1.2 Hz, 1H), 6.94 (d, J=1.2 Hz, 1H), 3.95 (s, 3H), 3.94 (s, 3H), 2.97 (s, 3H)

### Preparation of Intermediate Methyl 4-amino-3-methoxy-5-(methylamino)benzoate (3)

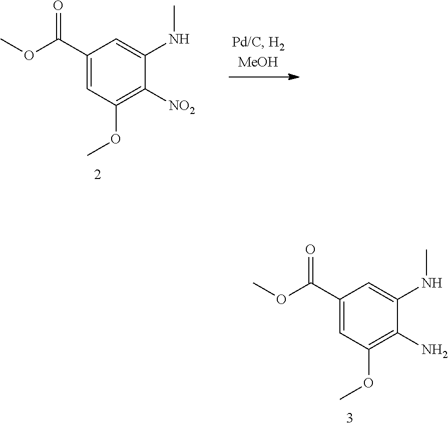

Equip a 10 mL 3-necked round bottom flask, addition funnel and thermometer, H₂ balloon. MeOH (4 mL) was charged to the three-necked round bottom flask, then methyl 3-methoxy-5-(methylamino)-4-nitrobenzoate 2 (500 mg, 2.08 mmol, 1 eq) was added to the mixture at 25° C. for 2 min under H₂ (15 psi). At 25° C., Pd/C (369 mg, 10%) was added in portions to the reaction mixture at 25° C. within 2 min under H₂ (15 psi). After the addition, the mixture was stirred at 25° C. for 1 hr under H₂ (15 psi). LC-MS showed starting material was consumed completely and one main peak with desired mass was detected. The suspension was filtered and the filter cake was washed with MeOH (5 mL*3). The combined filtrates were concentrated to dryness to give product.

LCMS: RT=0.269 min, MS cal.: 210.1, [M+H]⁺=211.0

¹H NMR (400 MHz, CHLOROFORM-d) δ=7.19 (d, J=1.6 Hz, 1H), 7.15-7.13 (d, J=1.6 Hz, 1H), 3.91 (s, 3H), 3.89 (s, 3H), 3.58 (br s, 2H), 2.92 (s, 3H)

### Preparation of Intermediate Methyl 4-(2-chloropropanamido)-3-methoxy-5-(methylamino)benzoate (4)

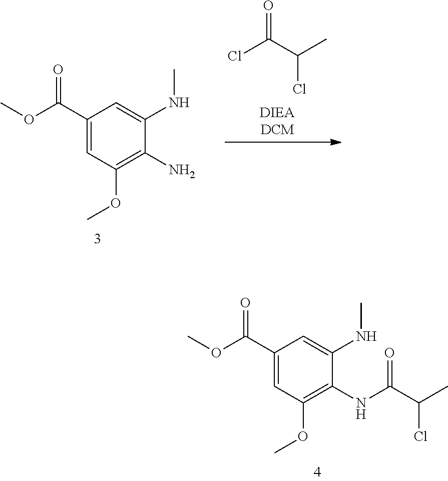

2-chloropropanoyl chloride (406 mg, 3.20 mmol, 310 μL, 1.05 eq) was added dropwise at 0° C. to methyl 4-amino-3-methoxy-5-(methylamino)benzoate 3 (0.64 g, 3.04 mmol, 1 eq) and DIEA (1.18 g, 9.13 mmol, 1.59 mL, 3 eq) in DCM under N₂. The reaction mixture was stirred for 2 h at 20° C. under N₂. LC-MS showed starting material was Consumed completely and one main peak with or desired mass was detected. The reaction mixture was added to 10 mL NH₄Cl and extracted by DCM (20*3 mL). Then organic phase was combined and washed by H₂O (10 mL), aq. brine (10 mL), dried with Na₂SO₄, filtered. The organic phase was concentrated under reduced pressure at 45° C. to provide product (790 mg, crude) as a yellow solid.

LCMS: RT=1.351 min, MS cal.: 300.1/302.1, [M+H]⁺=301.2/303.0

### Preparation of Intermediate Methyl 2-(1-chloroethyl)-4-methoxy-1-methyl-1H-benzo[d]imidazole-6-carboxylate (5)

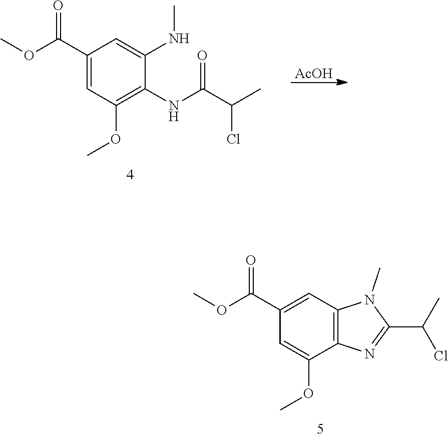 [SMILES: "AcOH" ; CNc1cc(C(=O)OC)cc(OC)c1NC(=O)C(C)Cl ; COC(=O)c1cc(OC)c2nc(C(C)Cl)n(C)c2c1]

A solution of methyl 4-(2-chloropropanamido)-3-methoxy-5-(methylamino)benzoate 4 (0.79 g, 2.63 mmol, 1 eq) in AcOH (15 mL) was stirred at 100° C. for 1h. LC-MS showed starting material was consumed completely and one main peak with desired mass was detected. The reaction was pour into the water (10 ml), then was added NaHCO₃ to adjust pH=5-6. The mixture was diluted with H₂O (30 mL), extracted with EtOAc (50 mL*3). The combined organic layer was washed with 20 mL H₂O, 20 mL brine, dried over Na₂SO₄, filtered and the filtrate was concentrated to give a crude product. The residue was purified by column chromatography (SiO₂, PE/EtOAc=10/1 to 0/1) to provide product (0.4 g, 1.41 mmol, 54% yield) as a yellow oil.

LCMS: RT=0.399 min, MS cal.: 282.1/284.1, [M+H]⁺=283.0/284.9

### Preparation of Intermediate Methyl 2-(1-(4-((S)-2-(5-chloropyridin-2-yl)-2-methylbenzo[d][1,3]dioxol-4-yl)piperidin-1-yl)ethyl)-4-methoxy-1-methyl-1H-benzo[d]imidazole-6-carboxylate (6)

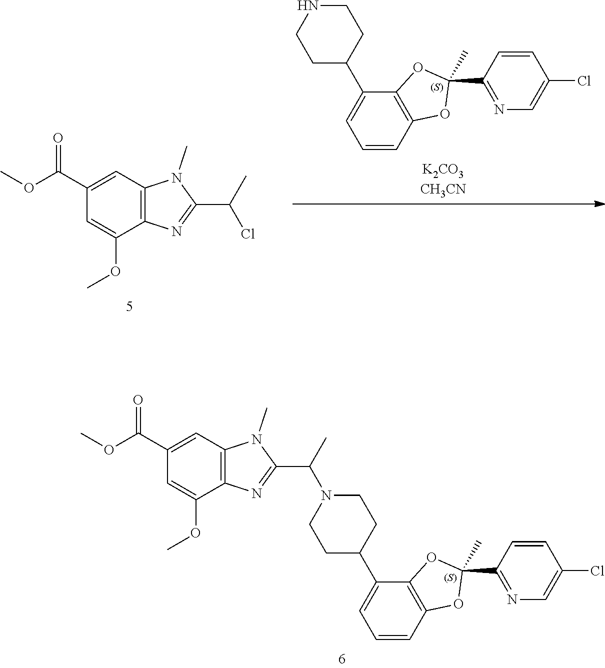

To a solution of methyl 2-(1-chloroethyl)-4-methoxy-1-methyl-1H-benzo[d]imidazole-6-carboxylate 5 (0.2 g, 707 μmol, 1 eq) in CH₃CN (2 mL) was added K₂CO₃ (293 mg, 2.12 mmol, 3 eq) and(S)-5-chloro-2-(2-methyl-4-(piperidin-4-yl)benzo[d][1,3]dioxol-2-yl)pyridine (257.42 mg, 778 μmol, 1.1 eq). The mixture was stirred at 60° C. for 4 h. LC-MS showed starting material was consumed completely and one main peak with desired mass was detected. The mixture was diluted with H₂O (20 mL), extracted with EtOAc (20 mL*3). The combined organic layer was washed with H₂O (20 mL), brine (20 Ml), dried over Na₂SO₄, filtered and the filtrate was concentrated to give a crude product. The residue was purified by column chromatography (SiO₂, PE/EtOAc=10/1 to 0/1) to provide product (0.24 g, 416 μmol, 59% yield) as a white solid.

LCMS: RT=0.462 min, MS cal.: 576.2/577.2, [M+H]⁺=577.2/578.2

### Preparation of Intermediate Methyl 2-((S)-1-(4-((S)-2-(5-chloropyridin-2-yl)-2-methylbenzo[d][1,3]dioxol-4-yl)piperidin-1-yl)ethyl)-4-methoxy-1-methyl-1H-benzo[d]imidazole-6-carboxylate (7) and Methyl 2-((R)-1-(4-((S)-2-(5-chloropyridin-2-yl)-2-methylbenzo[d][1,3]dioxol-4-yl)piperidin-1-yl)ethyl)-4-methoxy-1-methyl-1H-benzo[d]imidazole-6-carboxylate (8)

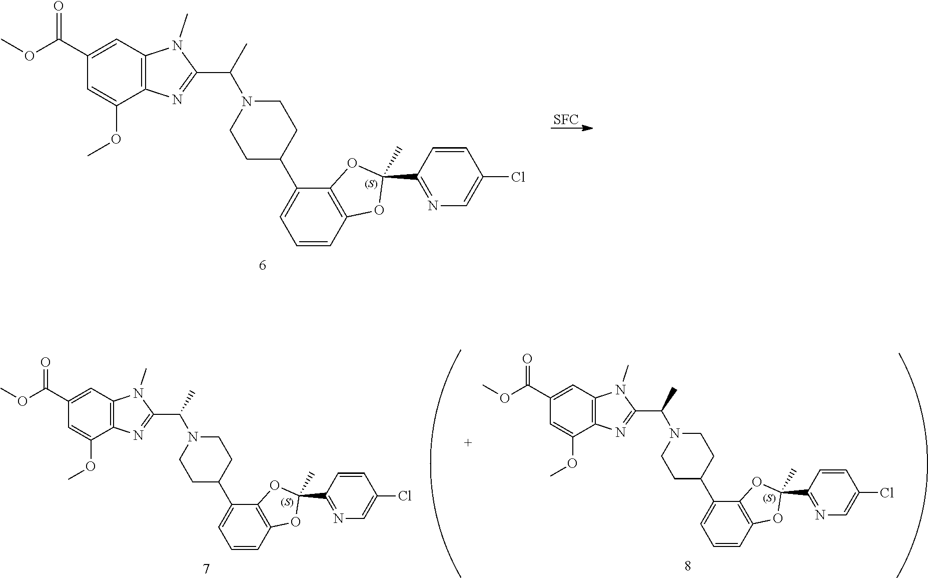

The residue of 6 was separated by SFC (column: DAICEL CHIRALPAK IG (250 mm*30 mm, 10 um); mobile phase: [CO₂-EtOH (0.1% NH₃H₂O)]; B %: 50%, isocratic elution mode) to provide 7 (120 mg, 208 μmol, 50% yield) and 8 (120 mg, 208 μmol, 50% yield) as white solids.

LCMS compound 7: RT=0.462 min, MS cal.: 576.2, [M+H]⁺=577.2

LCMS compound 8: RT=0.462 min, MS cal.: 576.2, [M+H]⁺=577.2

### Preparation of 2-((S)-1-(4-((S)-2-(5-Chloropyridin-2-yl)-2-methylbenzo[d][1,3]dioxol-4-yl)piperidin-1-yl)ethyl)-4-methoxy-1-methyl-1H-benzo[d]imidazole-6-carboxylic acid, Compound 96 (9)

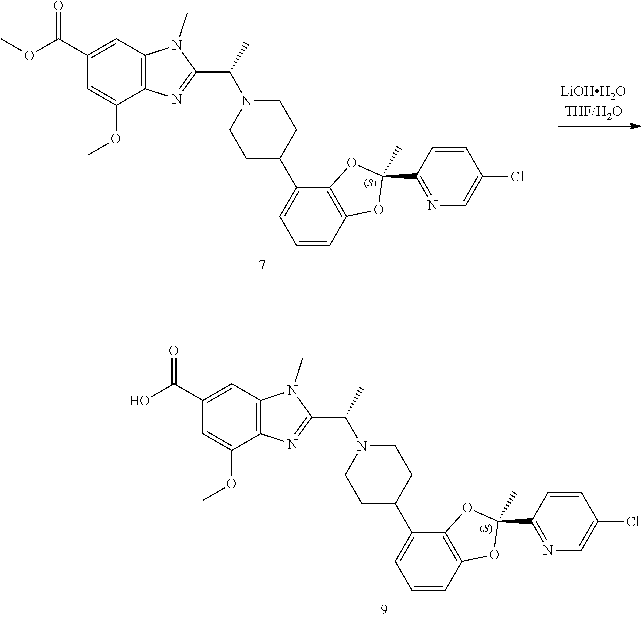

To a solution of methyl 2-((S)-1-(4-((S)-2-(5-chloropyridin-2-yl)-2-methylbenzo[d][1,3]dioxol-4-yl)piperidin-1-yl)ethyl)-4-methoxy-1-methyl-1H-benzo[d]imidazole-6-carboxylate 7 (100 mg, 173 μmol, 1 eq) in THF (0.7 mL) and H₂O (0.3 mL) was added LiOH·H₂O (14.5 mg, 347 μmol, 2 eq). The mixture was stirred at 20° C. for 12 h. LCMS showed 38% of starting material remained. The mixture was diluted with H₂O 1 mL.

The residue was purified by prep-HPLC (basic condition: column: Waters Xbridge Prep OBD C18 150*40 mm*10 um; mobile phase: [H₂O (10 mM NH₄HCO₃)-ACN]; gradient: 15%-65% B over 8.0 min) to provide product (26.4 mg, 46.4 μmol, 27% yield) as a white solid.

LCMS: RT=1.484 min, MS cal.: 562.2/563.2, [M+H]⁺=563.2/564.2.

QC:

LCMS: RT=2.566 min, MS cal.: 562.2/563.2, [M+H]⁺=563.2/564.2

HPLC: RT=10.340 min, purity: 99.61%

¹H NMR (400 MHz, DMSO-d₆) δ=8.71 (d, J=1.6 Hz, 1H), 8.02-7.96 (m, 1H), 7.77 (s, 1H), 7.58 (d, J=8.4 Hz, 1H), 7.26 (s, 1H), 6.80-6.70 (m, 3H), 4.32-4.24 (m, 1H), 3.94 (s, 3H), 3.89 (s, 3H), 3.05-2.97 (m, 1H), 2.63-2.53 (m, 3H), 2.26-2.16 (m, 1H), 1.99 (s, 3H), 1.85-1.72 (m, 2H), 1.69-1.60 (m, 1H), 1.58-1.49 (m, 1H), 1.45 (d, J=6.8 Hz, 3H).

### Example A38. —Preparation of Compound 97. 2-((R)-1-(4-((S)-2-(5-chloropyridin-2-yl)-2-methylbenzo[d][1,3]dioxol-4-yl)piperidin-1-yl)ethyl)-4-methoxy-1-methyl-1H-benzo[d]imidazole-6-carboxylic acid (Compound 97) (2)

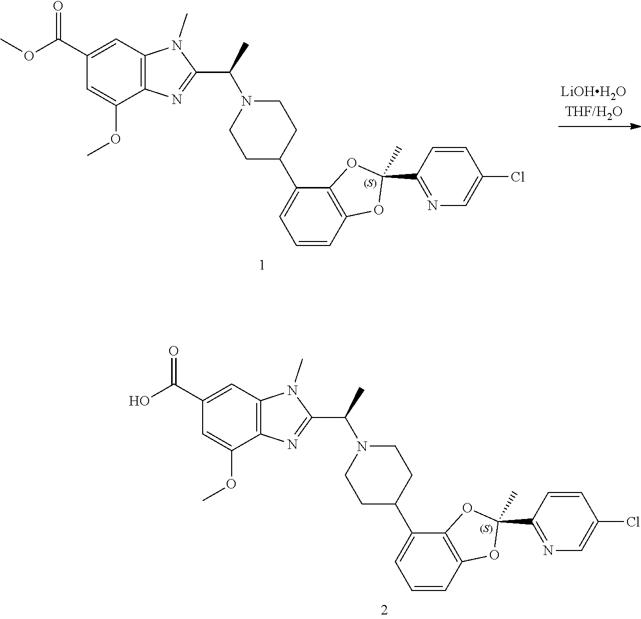

To a solution of methyl 2-((R)-1-(4-((S)-2-(5-chloropyridin-2-yl)-2-methylbenzo[d][1,3]dioxol-4-yl)piperidin-1-yl)ethyl)-4-methoxy-1-methyl-1H-benzo[d]imidazole-6-carboxylate 1 (100 mg, 173 μmol, 1 eq) in THF (0.7 mL) was added LiOH·H₂O (14.5 mg, 347 μmol, 2 eq) in H₂O (0.3 mL). The mixture was stirred at 25° C. for 12 h. HPLC showed starting material was consumed completely and one main peak with desired mass was detected. The reaction was filtered directly. The residue was purified by prep-HPLC (column: Waters Xbridge BEH C18 100*30 mm*10 um; mobile phase: [H₂O (10 mM NH₄HCO₃)-ACN]; gradient: 20%-60% B over 8.0 min) to provide product (28.4 mg, 50.4 μmol, 29% yield) as a white solid.

LCMS: RT=0.417 min, MS cal.: 562.2/563.2, [M+H]⁺=563.2/564.2

QC:

LCMS: RT=2.565 min, MS cal.: 562.2/563.2, [M+H]⁺=563.2/564.2

HPLC: RT=10.340 min, purity: 99.80%

¹H NMR (400 MHz, DMSO-d₆) δ=8.72-8.68 (d, J=2.4 Hz, 1H), 8.02-7.96 (m, 1H), 7.77 (s, 1H), 7.58 (d, J=8.8 Hz, 1H), 7.26 (s, 1H), 6.79-6.71 (m, 3H), 4.30-4.25 (m, 1H), 3.94 (s, 3H), 3.89 (s, 3H), 3.03-2.95 (m, 1H), 2.67-2.54 (m, 3H), 2.25-2.16 (m, 1H), 2.02 (s, 3H), 1.76-1.72 (m, 2H), 1.71-1.64 (m, 1H), 1.60-1.50 (m, 1H), 1.45 (d, J=6.8 Hz, 3H).

### Example A39. —Preparation of Compound 98. 2-((4-((S)-2-(5-chloropyridin-2-yl)-2-methylbenzo[d][1,3]dioxol-4-yl)piperidin-1-yl)methyl)-4-(methoxy-d₃)-1-(((S)-oxetan-2-yl)methyl)-1H-benzo[d]imidazole-6-carboxylic acid (Compound 98) (3)

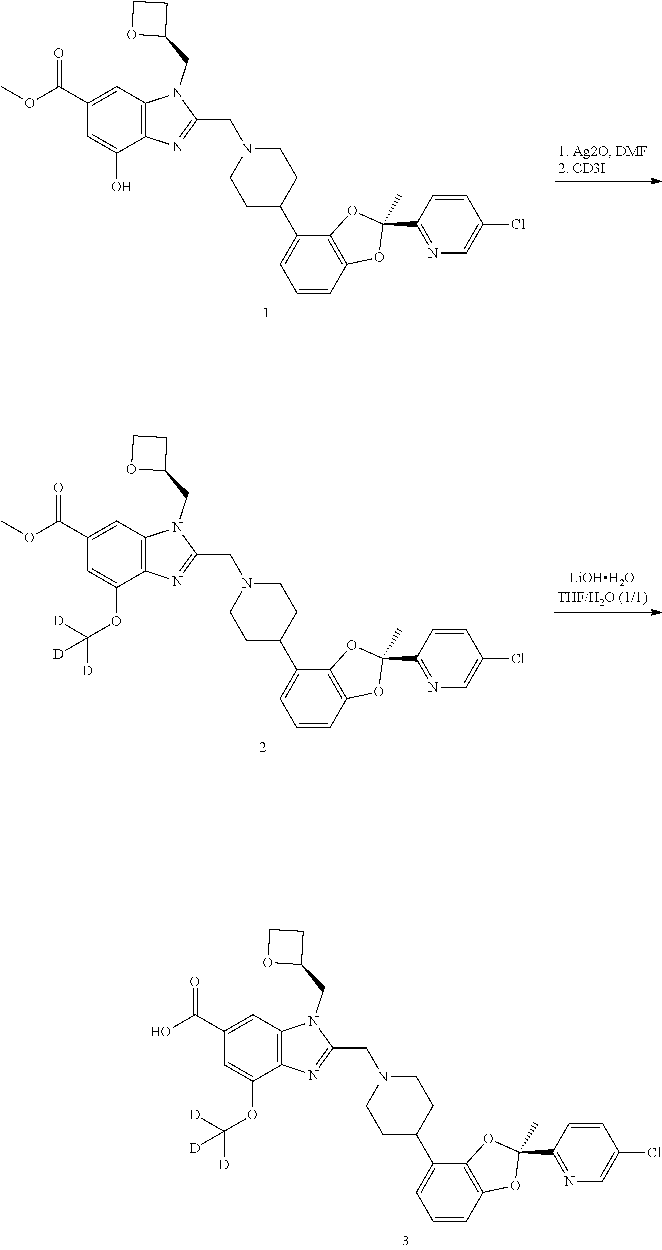 [SMILES: COC(=O)c1cc(O)c2nc(CN3CCC(c4cccc5c4O[C@@](C)(c4ccc(Cl)cn4)O5)CC3)n(C[C@@H]3CCO3)c2c1 ; [2H]C([2H])([2H])Oc1cc(C(=O)OC)cc2c1nc(CN1CCC(c3cccc4c3O[C@@](C)(c3ccc(Cl)cn3)O4)CC1)n2C[C@@H]1CCO1 ; [2H]C([2H])([2H])Oc1cc(C(=O)O)cc2c1nc(CN1CCC(c3cccc4c3O[C@@](C)(c3ccc(Cl)cn3)O4)CC1)n2C[C@@H]1CCO1]

### Preparation of Intermediate Methyl 2-((4-((S)-2-(5-chloropyridin-2-yl)-2-methylbenzo[d][1,3]dioxol-4-yl)piperidin-1-yl)methyl)-4-(methoxy-d₃)-1-(((S)-oxetan-2-yl)methyl)-1H-benzo[d]imidazole-6-carboxylate (2)

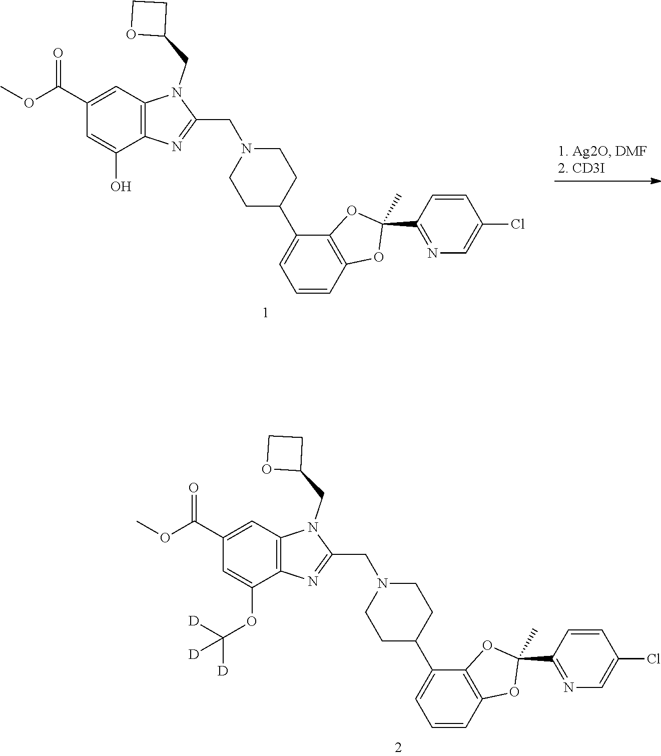 [SMILES: COC(=O)c1cc(O)c2nc(CN3CCC(c4cccc5c4O[C@@](C)(c4ccc(Cl)cn4)O5)CC3)n(C[C@@H]3CCO3)c2c1 ; [2H]C([2H])([2H])Oc1cc(C(=O)OC)cc2c1nc(CN1CCC(c3cccc4c3O[C@@](C)(c3ccc(Cl)cn3)O4)CC1)n2C[C@@H]1CCO1]

Equip a 25 mL three-necked round bottom flask, N₂ balloon. DMF (2 mL) was charged to the three-necked round bottom flask, then methyl 2-((4-((S)-2-(5-chloropyridin-2-yl)-2-methylbenzo[d][1,3]dioxol-4-yl)piperidin-1-yl)methyl)-4-hydroxy-1-(((S)-oxetan-2-yl)methyl)-1H-benzo[d]imidazole-6-carboxylate 1 (200 mg, 331 μmol, 1 eq) and Ag₂O (153 mg, 661 μmol, 2 eq) was added to the mixture at 25° C. for 0.5 h. At 25° C. (inner temperature), CD₃I (46.9 mg, 331 μmol, 1 eq) was added dropwise to the reaction mixture at 25° C. within 0.1 h. After the addition, the mixture was stirred at 25° C. for 12 h. LCMS showed starting material was consumed completely. TLC (Petroleum ether/Ethyl acetate=0/1, R_{f}=0.4) indicated starting material was consumed completely. After 12 h, the reaction mixture was added to H₂O (6 mL) at 25° C. The mixture was extracted by EtOAc (6 mL*3). Then organic phase was combined and washed by brine (6 mL), dried with Na₂SO₄, filtered. The organic phase was concentrated under reduced pressure at 35° C. to give a residue. The residue was purified by column chromatography (SiO₂, Petroleum ether/Ethyl acetate=3/1 to Ethyl acetate/Methanol=0/1) to provide product (200 mg, 321 μmol, 97% yield) as a pink solid.

LCMS: RT=1.481 min, MS cal.: 621.2, [M+H]⁺=622.1

¹H NMR (400 MHz, CHLOROFORM-d) δ=8.61 (d, J=2.0 Hz, 1H), 7.83 (s, 1H), 7.68 (dd, J=2.4, 8.4 Hz, 1H), 7.57 (d, J=8.4 Hz, 1H), 7.38 (d, J=1.2 Hz, 1H), 6.82-6.75 (m, 1H), 6.74-6.67 (m, 2H), 5.27-5.19 (m, 1H), 4.72-4.68 (m, 2H), 4.65-4.57 (m, 1H), 4.41-4.39 (m, 1H), 4.02-3.91 (m, 5H), 3.03-2.90 (m, 2H), 2.79-2.68 (m, 2H), 2.54-2.42 (m, 1H), 2.38-2.20 (m, 2H), 2.05 (s, 3H), 1.92-1.76 (m, 4H)

### Preparation of 2-((4-((S)-2-(5-Chloropyridin-2-yl)-2-methylbenzo[d][1,3]dioxol-4-yl)piperidin-1-yl)methyl)-4-(methoxy-d₃)-1-(((S)-oxetan-2-yl)methyl)-1H-benzo[d]imidazole-6-carboxylic acid Compound 98 (3)

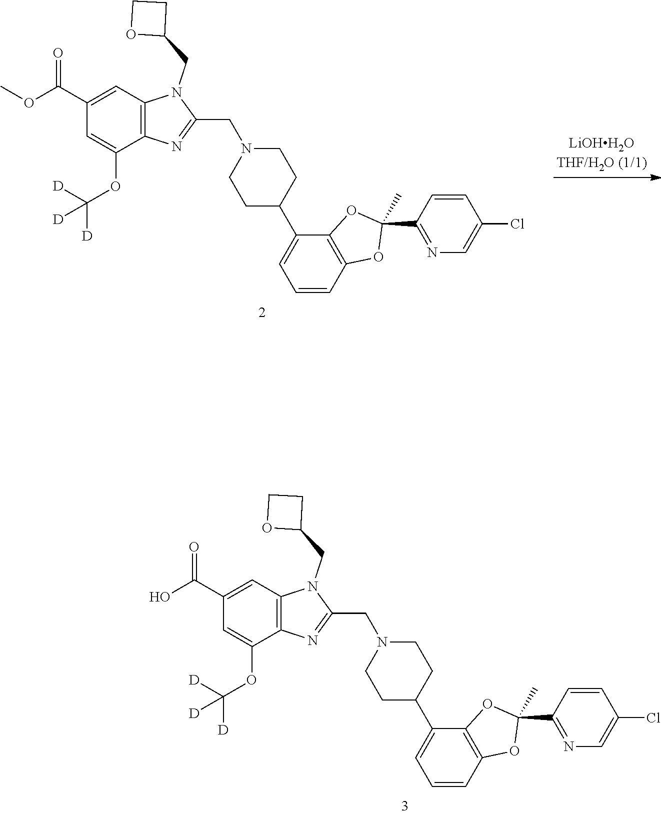 [SMILES: [2H]C([2H])([2H])Oc1cc(C(=O)OC)cc2c1nc(CN1CCC(c3cccc4c3O[C@@](C)(c3ccc(Cl)cn3)O4)CC1)n2C[C@@H]1CCO1 ; [2H]C([2H])([2H])Oc1cc(C(=O)O)cc2c1nc(CN1CCC(c3cccc4c3O[C@@](C)(c3ccc(Cl)cn3)O4)CC1)n2C[C@@H]1CCO1]

To a solution of methyl 2-((4-((S)-2-(5-chloropyridin-2-yl)-2-methylbenzo[d][1,3]dioxol-4-yl)piperidin-1-yl)methyl)-4-(methoxy-d₃)-1-(((S)-oxetan-2-yl)methyl)-1H-benzo[d]imidazole-6-carboxylate 2 (100 mg, 161 μmol, 1 eq) in THF (0.7 mL) was added LiOH·H₂O (10.1 mg, 241 μmol, 1.5 eq) in H₂O (0.3 mL). The mixture was stirred at 25° C. for 12 h. LCMS showed starting material was consumed completely. The reaction mixture was filtered. The filtrate was purified by prep-HPLC (column: Waters Xbridge Prep OBD C18 150*40 mm*10 um; mobile phase: [H₂O (10 mM NH₄HCO₃)-ACN]; gradient: 5%-55% B over 8.0 min) to provide product (28.1 mg, 44.7 μmol, 28% yield, 96.7% purity) as a white solid.

LCMS: RT=2.579 min, MS cal.: 607.2, [M+H]⁺=608.2

HPLC: RT=10.573 min, purity: 96.72%

¹H NMR (400 MHz, DMSO-d₆) δ=8.71 (d, J=2.4 Hz, 1H), 8.00 (dd, J=2.4, 8.4 Hz, 1H), 7.88 (d, J=1.2 Hz, 1H), 7.60 (d, J=8.4 Hz, 1H), 7.25 (d, J=1.2 Hz, 1H), 6.87-6.68 (m, 3H), 5.08-5.07 (m, 1H), 4.78-4.67 (m, 1H), 4.65-4.53 (m, 1H), 4.51-4.40 (m, 1H), 4.35-4.33 (m, 1H), 3.97-3.86 (d, J=13.2 Hz, 1H), 3.74 (d, J=13.2 Hz, 1H), 2.99 (d, J=10.4 Hz, 1H), 2.84 (d, J=11.2 Hz, 1H), 2.75-2.58 (m, 2H), 2.46-2.33 (m, 1H), 2.30-2.09 (m, 2H), 2.01 (s, 3H), 1.82-1.59 (m, 4H)

### Example A40. —Preparation of Compound 99. 2-((4-((S)-2-(5-Chloropyridin-2-yl)-2-methylbenzo[d][1,3]dioxol-4-yl)piperidin-1-yl)methyl)-1-(((S)-oxetan-2-yl)methyl)-7,8-dihydro-1H-[1,4]dioxino[2′,3′:3,4]benzo[1,2-d]imidazole-5-carboxylic acid (Compound 99) (4)

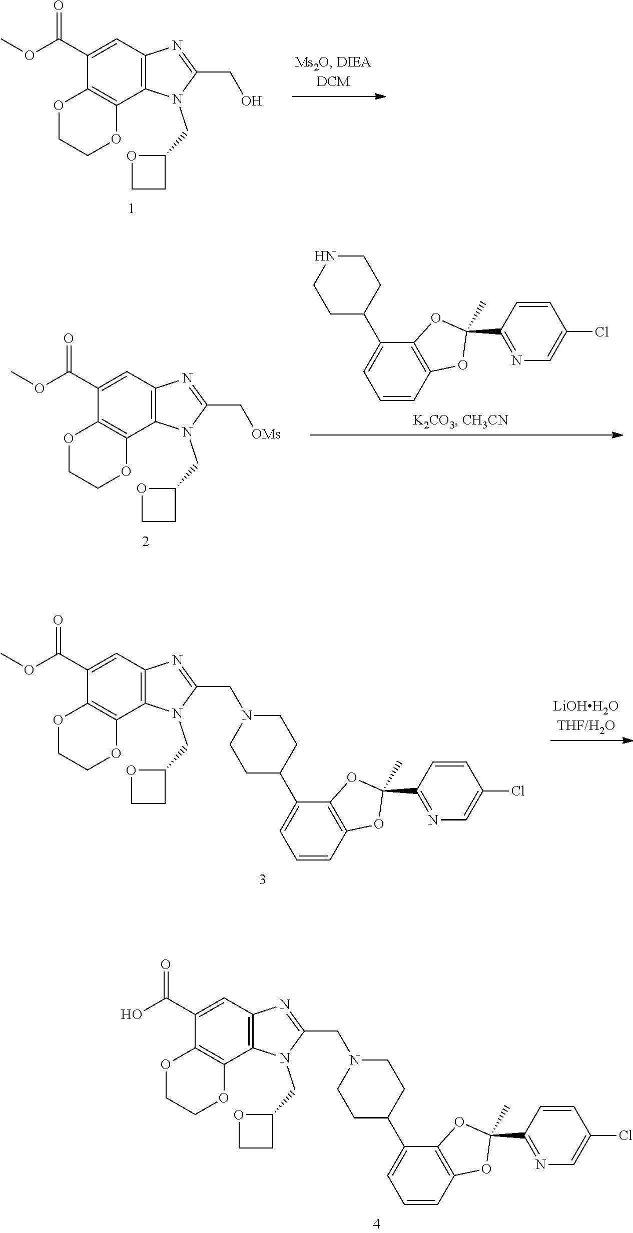 [SMILES: COC(=O)c1cc2nc(CO)n(C[C@@H]3CCO3)c2c2c1OCCO2 ; COC(=O)c1cc2nc(COS(C)(=O)=O)n(C[C@@H]3CCO3)c2c2c1OCCO2 ; C[C@]1(c2ccc(Cl)cn2)Oc2cccc(C3CCNCC3)c2O1 ; COC(=O)c1cc2nc(CN3CCC(c4cccc5c4O[C@@](C)(c4ccc(Cl)cn4)O5)CC3)n(C[C@@H]3CCO3)c2c2c1OCCO2 ; C[C@]1(c2ccc(Cl)cn2)Oc2cccc(C3CCN(Cc4nc5cc(C(=O)O)c6c(c5n4C[C@@H]4CCO4)OCCO6)CC3)c2O1]

### Preparation of Intermediate Methyl (S)-2-(((methylsulfonyl)oxy)methyl)-1-(oxetan-2-ylmethyl)-7,8-dihydro-1H-[1,4]dioxino[2′,3′:3,4]benzo[1,2-d]imidazole-5-carboxylate (2)

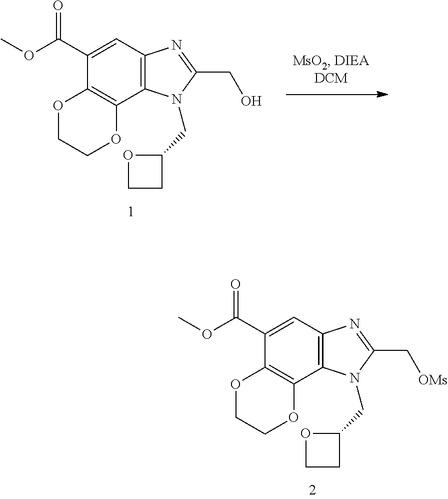 [SMILES: COC(=O)c1cc2nc(CO)n(C[C@@H]3CCO3)c2c2c1OCCO2 ; COC(=O)c1cc2nc(COS(C)(=O)=O)n(C[C@@H]3CCO3)c2c2c1OCCO2]

To a solution of methyl (S)-2-(hydroxymethyl)-1-(oxetan-2-ylmethyl)-7,8-dihydro-1H-[1,4]dioxino[2′,3′:3,4]benzo[1,2-d]imidazole-5-carboxylate 1 (70 mg, 209 μmol, 1 eq) in DCM (0.7 mL) was added Ms₂O (36.5 mg, 209 μmol, 1 eq) and DIEA (81.2 mg, 628 μmol, 109 μL, 3 eq). The mixture was stirred at 25° C. for 2 h. After monitoring, the reaction was not complete, then was added Ms₂O (7.29 mg, 41.9 μmol, 0.2 eq) and DIEA (81.2 mg, 628 μmol, 109 μL, 3 eq). The mixture was stirred at 25° C. for 24 h. LC-MS showed starting material was consumed completely and one main peak with desired mass was detected. The solvent was evaporated, then the crude was diluted with DCM (5 mL) and poured into FA (IM) solution (0.1 mL), and the pH was adjusted to 8. The residue was poured into water (10 mL). The aqueous phase was extracted with DCM (5 mL*3). The combined organic phase was washed with brine (10 mL*2), dried with anhydrous Na₂SO₄, filtered, and concentrated in vacuum to provide product (40 mg, crude) as a yellow oil.

LCMS: RT=0.346 min, MS cal.: 412.1, [M+H]⁺=413.1

### Preparation of Intermediate Methyl 2-((4-((S)-2-(5-chloropyridin-2-yl)-2-methylbenzo[d][1,3]dioxol-4-yl)piperidin-1-yl)methyl)-1-(((S)-oxetan-2-yl)methyl)-7,8-dihydro-1H-[1,4]dioxino[2′,3′:3,4]benzo[1,2-d]imidazole-5-carboxylate (3)

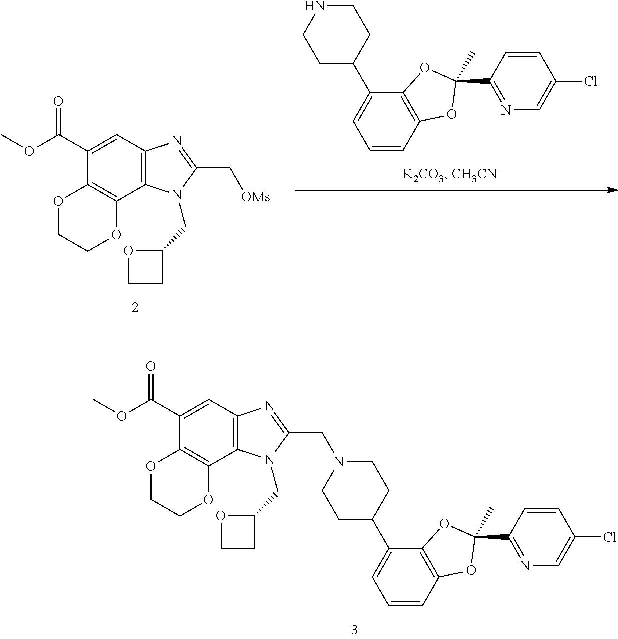 [SMILES: COC(=O)c1cc2nc(COS(C)(=O)=O)n(C[C@@H]3CCO3)c2c2c1OCCO2 ; C[C@]1(c2ccc(Cl)cn2)Oc2cccc(C3CCNCC3)c2O1 ; COC(=O)c1cc2nc(CN3CCC(c4cccc5c4O[C@@](C)(c4ccc(Cl)cn4)O5)CC3)n(C[C@@H]3CCO3)c2c2c1OCCO2]

To a solution of methyl (S)-2-(((methylsulfonyl)oxy)methyl)-1-(oxetan-2-ylmethyl)-7,8-dihydro-1H-[1,4]dioxino[2′,3′:3,4]benzo[1,2-d]imidazole-5-carboxylate 2 (40 mg, 97 μmol, 1 eq) in CH₃CN (0.4 mL) was added K₂CO₃ (40.2 mg, 291 μmol, 3 eq) and(S)-5-chloro-2-(2-methyl-4-(piperidin-4-yl)benzo[d][1,3]dioxol-2-yl)pyridine (35.3 mg, 107 μmol, 1.1 eq). The mixture was stirred at 60° C. for 4 h. LC-MS showed starting material was consumed completely and one main peak with desired mass was detected. The residue was poured into water (10 mL). The aqueous phase was extracted with EtOAc (5 mL*3). The combined organic phase was washed with brine (10 mL*2), dried with anhydrous Na₂SO₄, filtered, and concentrated in vacuum. The residue was purified by prep-HPLC (column: Waters Xbridge Prep OBD C18 150*40 mm*10 um; mobile phase: [H₂O (10 mM NH₄HCO₃)-ACN]; gradient: 50%-80% B over 8.0 min) to provide product (40 mg, 61.8 μmol, 64% yield) as a yellow oil.

LCMS: RT=0.429 min, MS cal.: 646.2/647.2, [M+H]⁺=647.2/649.1

### Preparation of 2-((4-((S)-2-(5-Chloropyridin-2-yl)-2-methylbenzo[d][1,3]dioxol-4-yl)piperidin-1-yl)methyl)-1-(((S)-oxetan-2-yl)methyl)-7,8-dihydro-1H-[1,4]dioxino[2′,3′:3,4]benzo[1,2-d]imidazole-5-carboxylic acid, Compound 99 (4)

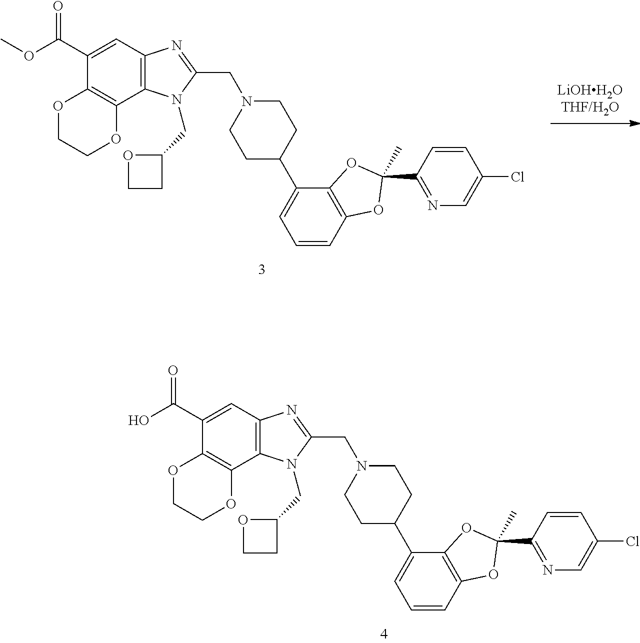 [SMILES: COC(=O)c1cc2nc(CN3CCC(c4cccc5c4O[C@@](C)(c4ccc(Cl)cn4)O5)CC3)n(C[C@@H]3CCO3)c2c2c1OCCO2 ; C[C@]1(c2ccc(Cl)cn2)Oc2cccc(C3CCN(Cc4nc5cc(C(=O)O)c6c(c5n4C[C@@H]4CCO4)OCCO6)CC3)c2O1]

To a solution of 3 (40 mg, 61.81 μmol, 1 eq) in THF (0.7 mL) was added LiOH·H₂O (3.11 mg, 74.2 μmol, 1.2 eq) in H₂O (0.3 mL). The mixture was stirred at 25° C. for 12 h. LC-MS showed starting material was consumed completely and one main peak with desired mass was detected. The reaction was filtered directly. The residue was purified by prep-HPLC (column: Waters Xbridge Prep OBD C18 150*40 mm*10 um; mobile phase: [H₂O (10 mM NH₄HCO₃)-ACN]; gradient: 30%-60% B over 8.0 min]; gradient: 25%-55% B over 8.0 min) to provide product (1.53 mg, 2.41 μmol, 4% yield) as a white solid.

LCMS: RT=0.415 min, MS cal.: 632.2/633.2, [M+H]⁺=633.3/634.2

QC:

LCMS: RT=2.594 min, MS cal.: 632.2/633.2, [M+H]⁺=633.2.3/634.2

HPLC: RT=10.195 min, purity: 99.62%

¹H NMR (400 MHz, DMSO-d6) δ=8.72 (d, J=2.4 Hz, 1H), 8.06-7.94 (dd, J=2.4, 8.4 Hz, 1H), 7.64-7.57 (d, J=8.4 Hz, 1H), 7.28 (s, 1H), 6.83-6.72 (m, 3H), 5.14-5.02 (m, 1H), 4.83-4.75 (m, 1H), 4.69-4.60 (m, 1H), 4.49-4.42 (m, 1H), 4.36-4.25 (m, 5H), 3.85-3.80 (m, 1H), 3.76-3.72 (m, 1H), 3.00-2.93 (m, 1H), 2.91-2.83 (m, 1H), 2.61-2.57 (m, 1H), 2.46-2.39 (m, 1H), 2.23-2.09 (m, 2H), 2.00 (s, 3H), 1.80-1.66 (m, 4H)

### Example A41. —Preparation of Compound 100. (S)-2-((4-(6-((4-Cyano-2-fluorobenzyl)oxy)pyridin-2-yl)piperidin-1-yl)methyl)-3-(oxetan-2-ylmethyl)-7,8-dihydro-3H-[1,4]dioxino[2′,3′:3,4]benzo[1,2-d]imidazole-5-carboxylic acid (Compound 100) (4)

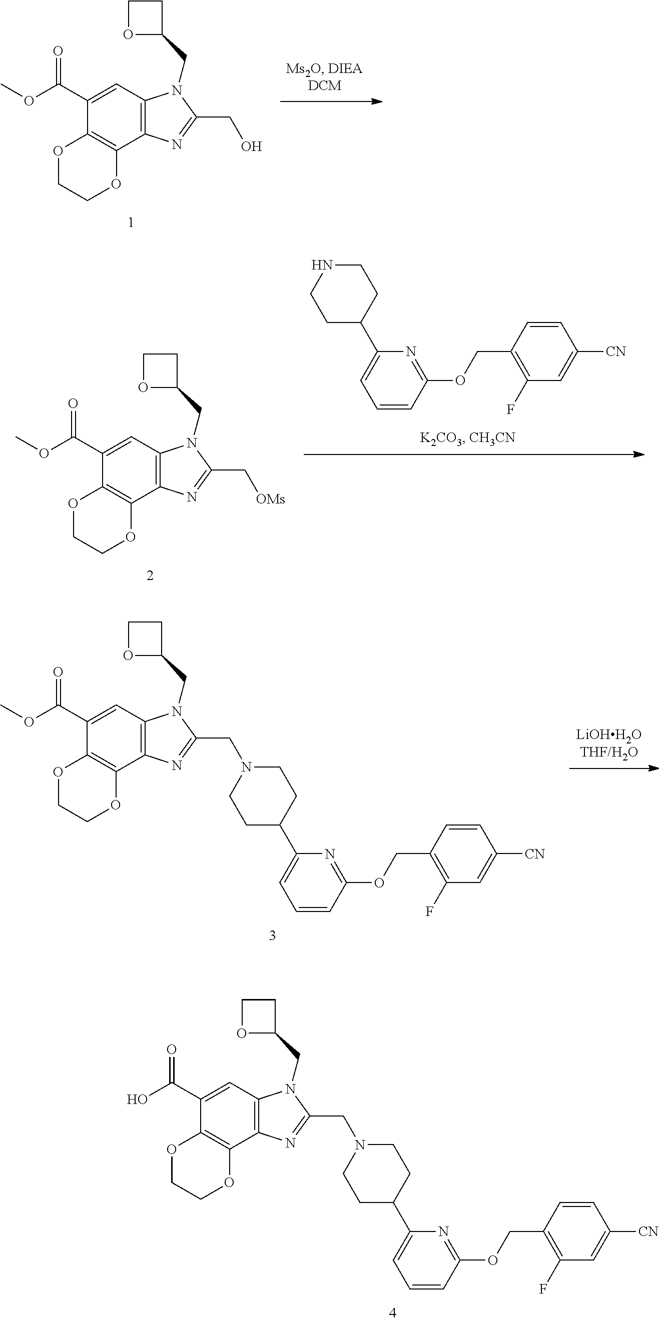 [SMILES: COC(=O)c1cc2c(nc(CO)n2C[C@@H]2CCO2)c2c1OCCO2 ; COC(=O)c1cc2c(nc(COS(C)(=O)=O)n2C[C@@H]2CCO2)c2c1OCCO2 ; N#Cc1ccc(COc2cccc(C3CCNCC3)n2)c(F)c1 ; COC(=O)c1cc2c(nc(CN3CCC(c4cccc(OCc5ccc(C#N)cc5F)n4)CC3)n2C[C@@H]2CCO2)c2c1OCCO2 ; N#Cc1ccc(COc2cccc(C3CCN(Cc4nc5c6c(c(C(=O)O)cc5n4C[C@@H]4CCO4)OCCO6)CC3)n2)c(F)c1]

### Preparation of Intermediate Methyl (S)-2-(((methylsulfonyl)oxy)methyl)-3-(oxetan-2-ylmethyl)-7,8-dihydro-3H-[1,4]dioxino[2′,3′:3,4]benzo[1,2-d]imidazole-5-carboxylate (2)

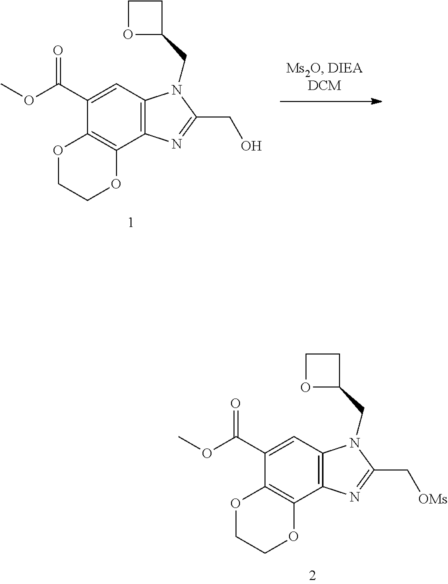 [SMILES: COC(=O)c1cc2c(nc(CO)n2C[C@@H]2CCO2)c2c1OCCO2 ; COC(=O)c1cc2c(nc(COS(C)(=O)=O)n2C[C@@H]2CCO2)c2c1OCCO2]

To a solution of methyl (S)-2-(hydroxymethyl)-3-(oxetan-2-ylmethyl)-7,8-dihydro-3H-[1,4]dioxino[2′,3′:3,4]benzo[1,2-d]imidazole-5-carboxylate 1 (50 mg, 149.56 μmol, 1 eq) in DCM (0.5 mL) was added Ms₂O (39.1 mg, 224.3 μmol, 1.5 eq) and DIEA (58 mg, 449 μmol, 78.2 μL, 3 eq). The mixture was stirred at 25° C. for 2 h. LC-MS showed starting material was consumed completely and desired mass was detected. The mixture was diluted with H₂O (5 mL), and extracted by DCM (5 mL*2). Then organic phase was combined and washed by H₂O (3 mL*2), dried with Na₂SO₄, filtered. The organic phase was concentrated under reduced pressure at 40° C. to provide product (60 mg, 145 μmol, 97% yield) as a white solid.

LCMS: RT=0.559 min, MS cal.: 412.1, [M+H]⁺=412.4

¹H NMR (400 MHz, CHLOROFORM-d) δ=9.96-9.69 (m, 1H), 7.62-7.42 (m, 1H), 7.27 (s, 1H), 5.75-5.66 (m, 1H), 5.64-5.53 (m, 1H), 5.23-5.11 (m, 1H), 5.09-4.97 (m, 1H), 4.69-4.53 (m, 3H), 4.53-4.47 (m, 3H), 4.47-4.40 (m, 4H), 4.39-4.27 (m, 2H), 3.93-3.91 (m, 1H), 3.77-3.61 (m, 1H), 3.10 (s, 3H), 2.78 (s, 3H), 2.45-2.35 (m, 1H)

### Preparation of Intermediate Methyl (S)-2-((4-(6-((4-cyano-2-fluorobenzyl)oxy)pyridin-2-yl)piperidin-1-yl)methyl)-3-(oxetan-2-ylmethyl)-7,8-dihydro-3H-[1,4]dioxino[2′,3′:3,4]benzo[1,2-d]imidazole-5-carboxylate (3)

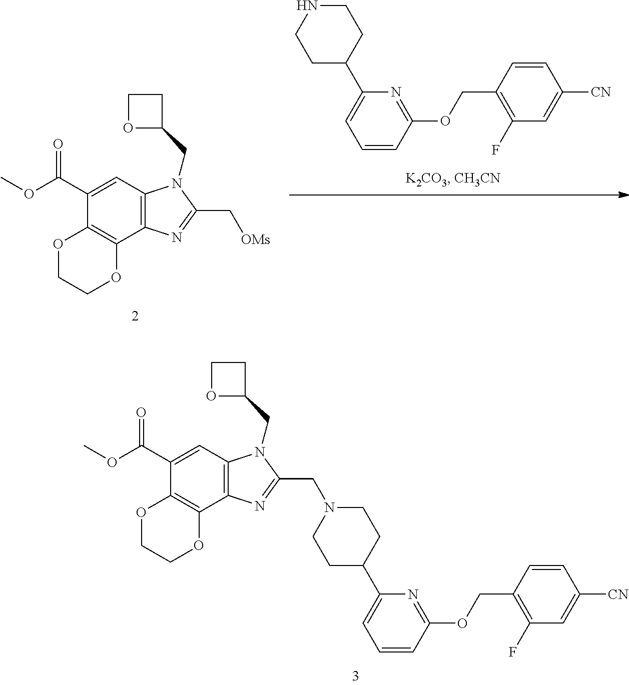 [SMILES: COC(=O)c1cc2c(nc(COS(C)(=O)=O)n2C[C@@H]2CCO2)c2c1OCCO2 ; N#Cc1ccc(COc2cccc(C3CCNCC3)n2)c(F)c1 ; COC(=O)c1cc2c(nc(CN3CCC(c4cccc(OCc5ccc(C#N)cc5F)n4)CC3)n2C[C@@H]2CCO2)c2c1OCCO2]

To a solution of methyl (S)-2-(((methylsulfonyl)oxy)methyl)-3-(oxetan-2-ylmethyl)-7,8-dihydro-3H-[1,4]dioxino[2′,3′:3,4]benzo[1,2-d]imidazole-5-carboxylate 2 (60 mg, 145 μmol, 1 eq) in ACN (0.6 mL) was added K₂CO₃ (60.3 mg, 436 μmol, 3 eq) and 3-fluoro-4-(((6-(piperidin-4-yl)pyridin-2-yl)oxy)methyl)benzonitrile (49.8 mg, 160 μmol, 1.1 eq). The mixture was stirred at 60° C. for 12 h. LC-MS showed starting material was consumed completely and desired mass was detected. The mixture was diluted with H₂O (5 mL), and extracted by EtOAc (5 mL*2). Then organic phase was combined and washed by H₂O (5 mL*2), dried with Na₂SO₄, filtered. The organic phase was concentrated under reduced pressure at 40° C. to give a residue. The residue was purified by column chromatography (SiO₂, Petroleum ether/Ethyl acetate=50/1 to 0/1) to provide product (43 mg, 68.5 μmol, 47% yield) as a white solid.

LCMS: RT=0.692 min, MS cal.: 627.2, [M+H]⁺=628.2

¹H NMR (400 MHz, DMSO-d6) δ=7.89 (d, J=10.4 Hz, 1H), 7.73-7.67 (m, 2H), 7.64 (t, J=7.6 Hz, 1H), 7.56 (s, 1H), 6.88 (d, J=7.6 Hz, 1H), 6.72 (d, J=8.0 Hz, 1H), 5.47 (s, 2H), 5.11-5.00 (m, 1H), 4.73-4.64 (m, 1H), 4.60-4.52 (m, 1H), 4.52-4.42 (m, 1H), 4.40-4.32 (m, 3H), 4.30 (br d, J=2.8 Hz, 2H), 3.87 (d, J=13.6 Hz, 1H), 3.79 (s, 3H), 3.72 (d, J=13.6 Hz, 1H), 2.95 (br d, J=11.2 Hz, 1H), 2.82 (br d, J=11.2 Hz, 1H), 2.69 (s, 3H), 2.61-2.56 (m, 1H), 2.44-2.37 (m, 1H), 2.24-2.09 (m, 3H), 1.93-1.87 (m, 1H), 1.81-1.57 (m, 4H)

### Preparation of (S)-2-((4-(6-((4-Cyano-2-fluorobenzyl)oxy)pyridin-2-yl)piperidin-1-yl)methyl)-3-(oxetan-2-ylmethyl)-7,8-dihydro-3H-[1,4]dioxino[2′,3′:3,4]benzo[1,2-d]imidazole-5-carboxylic acid, Compound 100 (4)

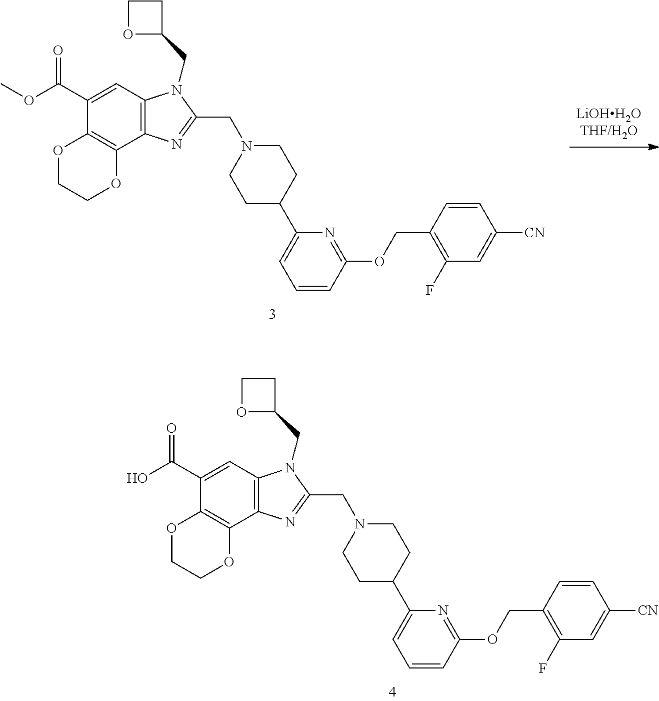 [SMILES: COC(=O)c1cc2c(nc(CN3CCC(c4cccc(OCc5ccc(C#N)cc5F)n4)CC3)n2C[C@@H]2CCO2)c2c1OCCO2 ; N#Cc1ccc(COc2cccc(C3CCN(Cc4nc5c6c(c(C(=O)O)cc5n4C[C@@H]4CCO4)OCCO6)CC3)n2)c(F)c1]

To a solution of methyl (S)-2-((4-(6-((4-cyano-2-fluorobenzyl)oxy)pyridin-2-yl)piperidin-1-yl)methyl)-3-(oxetan-2-ylmethyl)-7,8-dihydro-3H-[1,4]dioxino[2′,3′:3,4]benzo[1,2-d]imidazole-5-carboxylate 3 (43 mg, 68.5 μmol, 1 eq) in H₂O (0.13 mL) and THF (0.3 mL) was added LiOH·H₂O (4.31 mg, 103 μmol, 1.5 eq). The mixture was stirred at 25° C. for 12 h. LC-MS showed starting material was consumed completely and desired mass was detected. The mixture was concentrated under reduced pressure to give a residue. The crude product was purified by reversed-phase HPLC (column: Waters Xbridge Prep OBD C18 150*40 mm*10 um; mobile phase: [H₂O (10 mM NH₄HCO₃)-ACN]; gradient: 20%-60% B over 8.0 min) to provide product (29.2 mg, 47.0 μmol, 68% yield, 98% purity) as a white solid.

LCMS: RT=1.452 min, MS cal.: 613.2, [M+H]⁺=614.2

HPLC: RT=9.634 min, purity: 98%

¹H NMR (400 MHz, METHANOL-d4) δ=7.71-7.64 (m, 1H), 7.63-7.51 (m, 4H), 6.85 (d, J=7.2 Hz, 1H), 6.69 (d, J=8.0 Hz, 1H), 5.52 (s, 2H), 5.23 (dq, J=2.4, 7.2 Hz, 1H), 4.75 (d, J=6.8 Hz, 1H), 4.66-4.61 (m, 2H), 4.49-4.41 (m, 3H), 4.40-4.35 (m, 2H), 4.14-4.06 (d, J=14.0 Hz, 1H), 4.05-3.97 (d, J=14.0 Hz, 1H), 3.16-3.14 (m, 1H), 3.08-3.06 (m, 1H), 2.84-2.63 (m, 2H), 2.57-2.40 (m, 3H), 1.92-1.82 (m, 4H)

### Example A42. —Preparation of Compound 101. (S)-2-((4-(6-((4-Cyano-2-fluorobenzyl)oxy)pyridin-2-yl)piperidin-1-yl)methyl)-1-(oxetan-2-ylmethyl)-7,8-dihydro-1H-[1,4]dioxino[2′,3′:3,4]benzo[1,2-d]imidazole-5-carboxylic acid (Compound 101) (4)

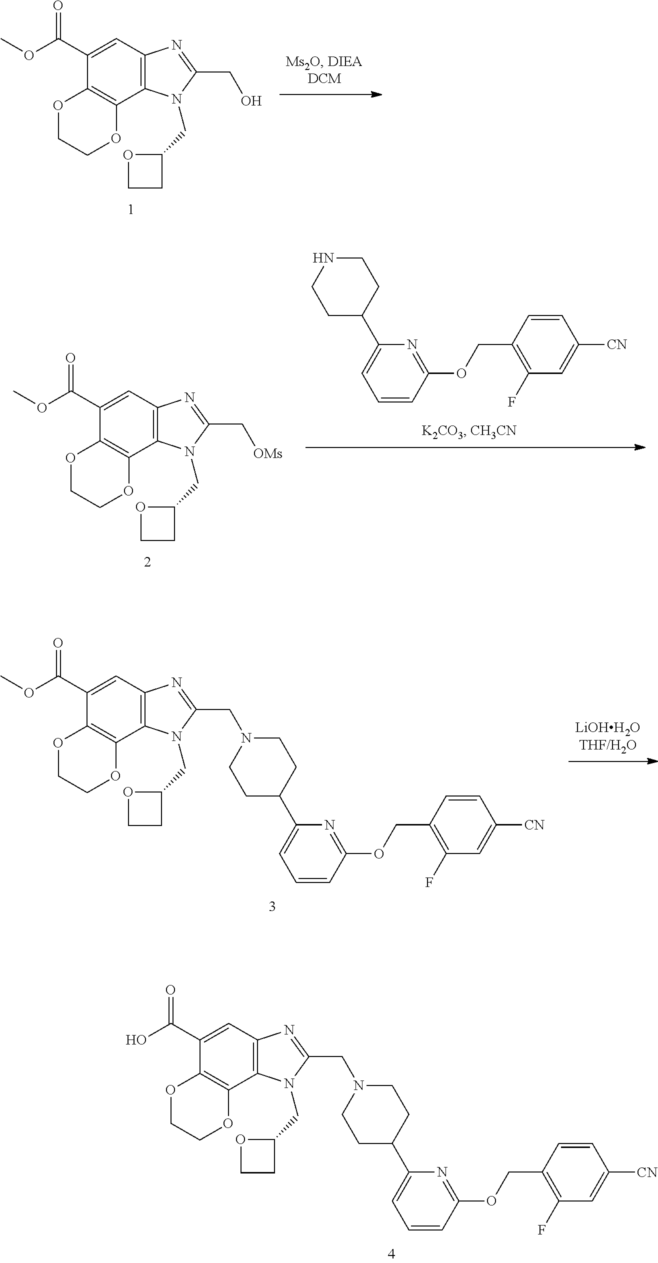 [SMILES: COC(=O)c1cc2nc(CO)n(C[C@@H]3CCO3)c2c2c1OCCO2 ; COC(=O)c1cc2nc(COS(C)(=O)=O)n(C[C@@H]3CCO3)c2c2c1OCCO2 ; N#Cc1ccc(COc2cccc(C3CCNCC3)n2)c(F)c1 ; COC(=O)c1cc2nc(CN3CCC(c4cccc(OCc5ccc(C#N)cc5F)n4)CC3)n(C[C@@H]3CCO3)c2c2c1OCCO2 ; N#Cc1ccc(COc2cccc(C3CCN(Cc4nc5cc(C(=O)O)c6c(c5n4C[C@@H]4CCO4)OCCO6)CC3)n2)c(F)c1]

### Preparation of Intermediate Methyl (S)-2-(((methylsulfonyl)oxy)methyl)-1-(oxetan-2-ylmethyl)-7,8-dihydro-1H-[1,4]dioxino[2′,3′:3,4]benzo[1,2-d]imidazole-5-carboxylate (2)

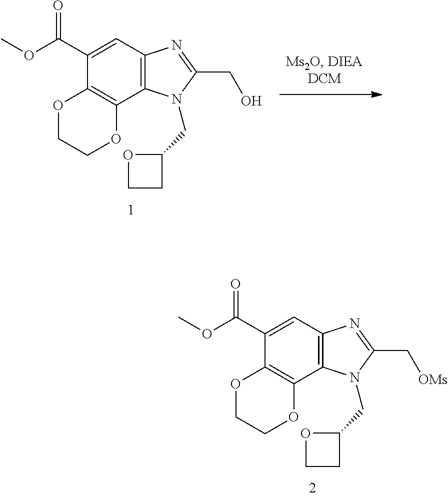 [SMILES: COC(=O)c1cc2nc(CO)n(C[C@@H]3CCO3)c2c2c1OCCO2 ; COC(=O)c1cc2nc(COS(C)(=O)=O)n(C[C@@H]3CCO3)c2c2c1OCCO2]

To a solution of methyl (S)-2-(hydroxymethyl)-1-(oxetan-2-ylmethyl)-7,8-dihydro-1H-[1,4]dioxino[2′,3′:3,4]benzo[1,2-d]imidazole-5-carboxylate 1 (150 mg, 449 μmol, 1 eq) in DCM (0.3 mL) was added Ms₂O (117 mg, 673 μmol, 1.5 eq) and DIEA (174 mg, 1.35 mmol, 234 μL, 3 eq). The mixture was stirred at 25° C. for 2 h. LC-MS showed starting material was consumed completely and one main peak with desired mass was detected. The solvent was evaporated, then the crude was diluted with DCM (5 mL) and added with FA (1M, 0.1 mL) and the pH was adjusted to 8. The residue was poured into water (10 mL). The aqueous phase was extracted with DCM (5 mL*3). The combined organic phase was washed with brine (10 mL*2), dried with anhydrous Na₂SO₄, filtered, and concentrated in vacuum to provide product (140 mg, 339 μmol, 76% yield) as a yellow oil.

LCMS: RT=0.337 min, MS cal.: 412.1, [M+H]⁺=413.0

### Preparation of Intermediate Methyl (S)-2-((4-(6-((4-cyano-2-fluorobenzyl)oxy)pyridin-2-yl)piperidin-1-yl)methyl)-1-(oxetan-2-ylmethyl)-7,8-dihydro-1H-[1,4]dioxino[2′,3′:3,4]benzo[1,2-d]imidazole-5-carboxylate (3)

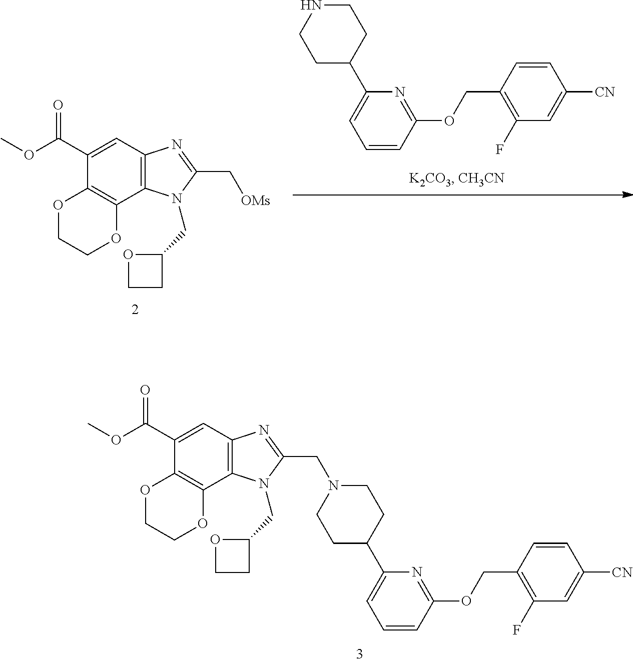 [SMILES: COC(=O)c1cc2nc(COS(C)(=O)=O)n(C[C@@H]3CCO3)c2c2c1OCCO2 ; N#Cc1ccc(COc2cccc(C3CCNCC3)n2)c(F)c1 ; COC(=O)c1cc2nc(CN3CCC(c4cccc(OCc5ccc(C#N)cc5F)n4)CC3)n(C[C@@H]3CCO3)c2c2c1OCCO2]

To a solution of methyl (S)-2-(((methylsulfonyl)oxy)methyl)-1-(oxetan-2-ylmethyl)-7,8-dihydro-1H-[1,4]dioxino[2′,3′:3,4]benzo[1,2-d]imidazole-5-carboxylate 2 (140 mg, 339 μmol, 1 eq) in CH₃CN (0.2 mL) was added K₂CO₃ (141 mg, 1.02 mmol, 3 eq) and 3-fluoro-4-(((6-(piperidin-4-yl)pyridin-2-yl)oxy)methyl)benzonitrile (116 mg, 373 μmol, 1.1 eq). The mixture was stirred at 60° C. for 4 h. LC-MS showed starting material was consumed completely and one main peak with desired mass was detected. The residue was poured into water (10 mL). The mixture was extracted with EtOAc (5 mL*3). The combined organic phase was washed with brine (10 mL*2), dried with anhydrous Na₂SO₄, filtered, and concentrated in vacuum. The residue was purified by column chromatography (SiO₂, PE/EtOAc=10/1 to 0/1) to provide product (160 mg, 255 μmol, 75% yield) as a yellow oil.

### Preparation of (S)-2-((4-(6-((4-Cyano-2-fluorobenzyl)oxy)pyridin-2-yl)piperidin-1-yl)methyl)-1-(oxetan-2-ylmethyl)-7,8-dihydro-1H-[1,4]dioxino[2′,3′:3,4]benzo[1,2-d]imidazole-5-carboxylic acid, Compound 101 (4)

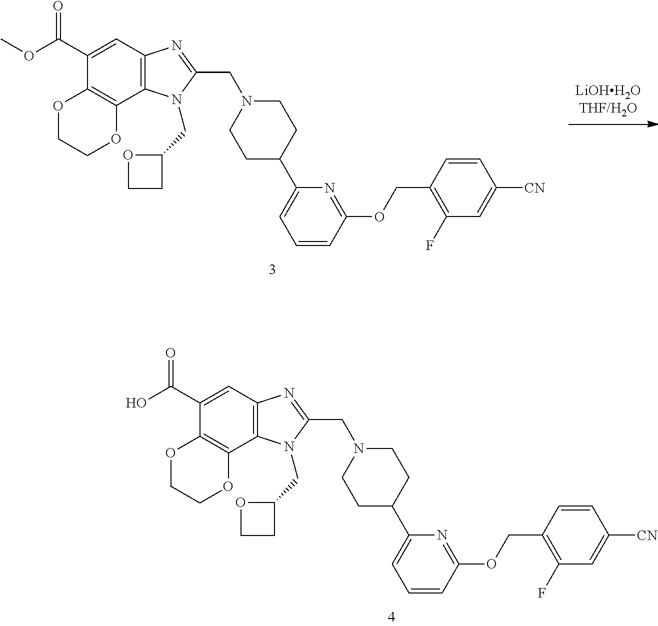 [SMILES: COC(=O)c1cc2nc(CN3CCC(c4cccc(OCc5ccc(C#N)cc5F)n4)CC3)n(C[C@@H]3CCO3)c2c2c1OCCO2 ; N#Cc1ccc(COc2cccc(C3CCN(Cc4nc5cc(C(=O)O)c6c(c5n4C[C@@H]4CCO4)OCCO6)CC3)n2)c(F)c1]

Methyl (S)-2-((4-(6-((4-cyano-2-fluorobenzyl)oxy)pyridin-2-yl)piperidin-1-yl)methyl)-1-(oxetan-2-ylmethyl)-7,8-dihydro-1H-[1,4]dioxino[2′,3′:3,4]benzo[1,2-d]imidazole-5-carboxylate 3 (0.127 g, 207 μmol) and LiOH·H₂O (13.0 mg, 311 μmol, 1.5 eq) in THF and H₂O (0.39 mL) were stirred at 25° C. for 12 h. LC-MS showed starting material was consumed completely and one main peak with desired mass was detected. The reaction was filtered directly. The residue was purified by prep-HPLC (column: Waters Xbridge BEH C18 100*30 mm*10 um; mobile phase: [H₂O (10 mM NH₄HCO₃)-ACN]; gradient: 25%-55% B over 8.0 min) to provide product (28 mg, 45.6 μmol, 22% yield) as a white solid.

LCMS: RT=0.405 min, MS cal.: 632.2/633.2, [M+H]⁺=614.2/615.2

QC:

LCMS: RT=2.604 min, MS cal.: 632.2/633.2, [M+H]⁺=614.3/615.3 HPLC: RT=9.776 min, purity: 93.41%

1H NMR (400 MHz, DMSO-d6) δ=7.92-7.85 (m, 1H), 7.73-7.68 (m, 2H), 7.67-7.61 (m, 1H), 7.47 (s, 1H), 6.92-6.87 (d, J=7.2 Hz, 1H), 6.75-6.70 (d, J=8.0 Hz, 1H), 5.46 (s, 2H), 5.15-5.04 (m, 1H), 4.90-4.81 (m, 1H), 4.74-4.66 (m, 1H), 4.52-4.44 (m, 1H), 4.41-4.29 (m, 5H), 3.88-3.81 (d, J=13.6 Hz, 1H), 3.78-3.72 (d, J=13.6 Hz, 1H), 2.97-2.80 (m, 2H), 2.70-2.64 (m, 1H), 2.63-2.56 (m, 1H), 2.46-2.38 (m, 1H), 2.22-2.11 (m, 2H), 1.83-1.62 (m, 4H).

### Example A43. —Preparation of Compound 102. (S)-2-(1-(4-(6-((4-Cyano-2-fluorobenzyl)oxy)pyridin-2-yl)piperidin-1-yl)ethyl)-4-methoxy-1-methyl-1H-benzo[d]imidazole-6-carboxylic acid (Compound 102) (5)

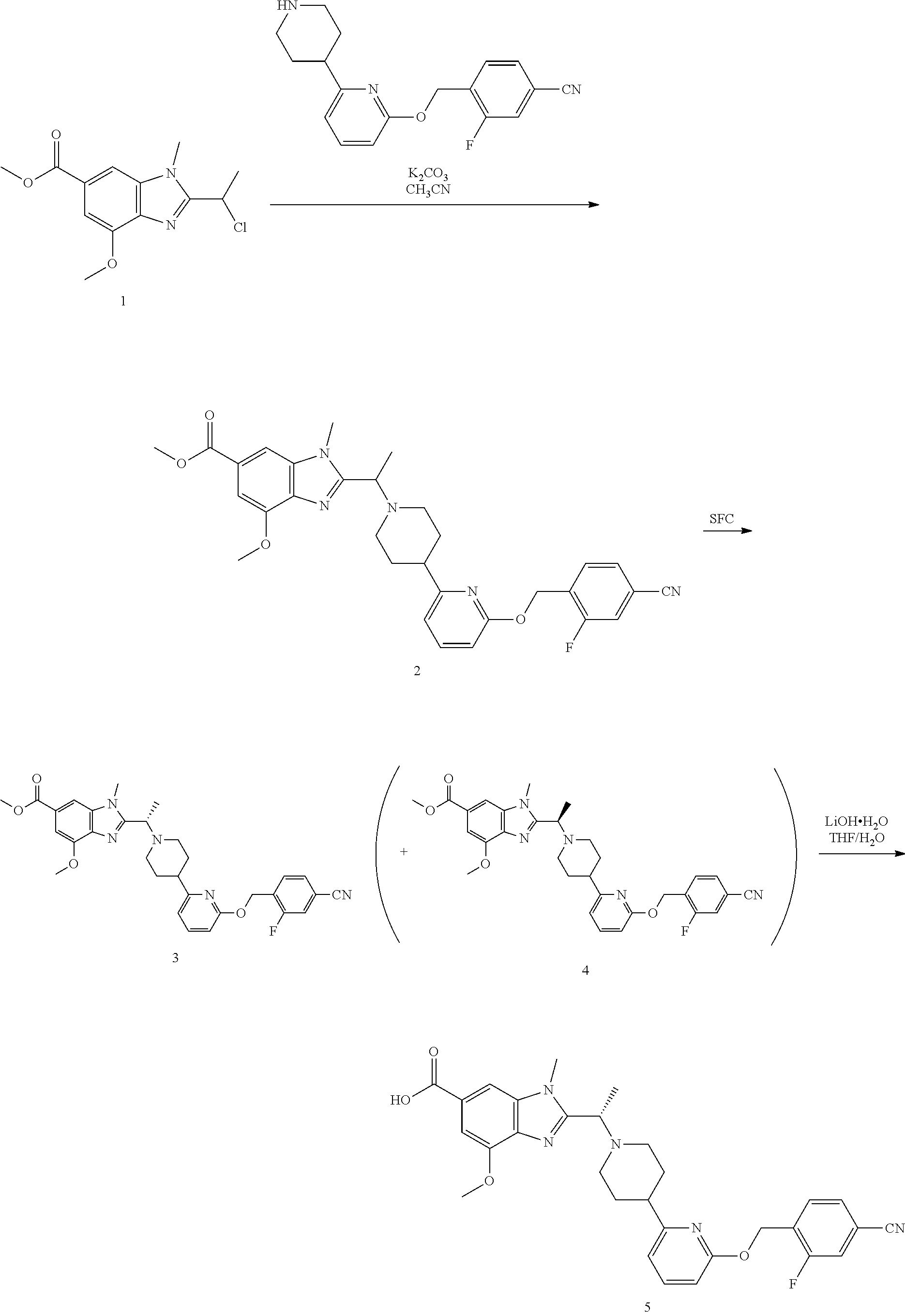

### Preparation of Intermediate Methyl 2-(1-(4-(6-((4-cyano-2-fluorobenzyl)oxy)pyridin-2-yl)piperidin-1-yl)ethyl)-4-methoxy-1-methyl-1H-benzo[d]imidazole-6-carboxylate (2)

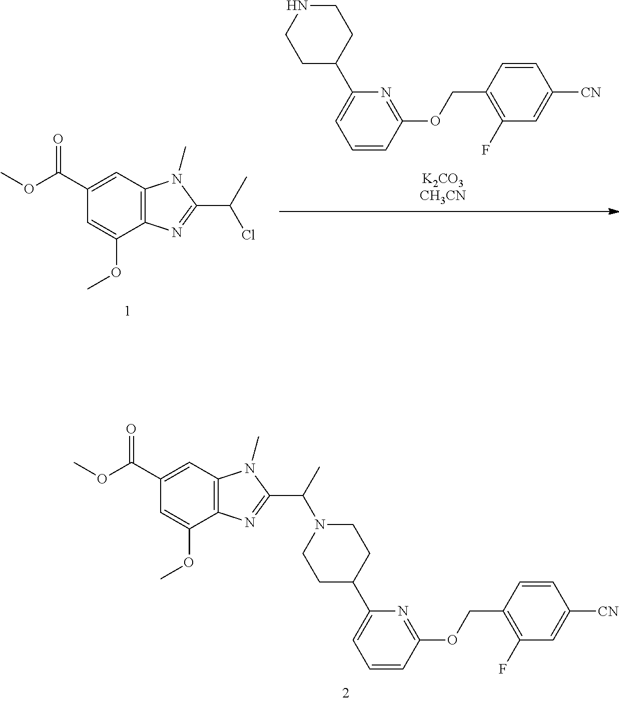 [SMILES: COC(=O)c1cc(OC)c2nc(C(C)Cl)n(C)c2c1 ; N#Cc1ccc(COc2cccc(C3CCNCC3)n2)c(F)c1 ; COC(=O)c1cc(OC)c2nc(C(C)N3CCC(c4cccc(OCc5ccc(C#N)cc5F)n4)CC3)n(C)c2c1]

A mixture of methyl 2-(1-chloroethyl)-4-methoxy-1-methyl-1H-benzo[d]imidazole-6-carboxylate 1 (120 mg, 424 μmol, 1 eq) in CH₃CN (2 mL) was added 3-fluoro-4-(((6-(piperidin-4-yl)pyridin-2-yl)oxy)methyl)benzonitrile (145 mg, 467 μmol, 1.1 eq) and K₂CO₃ (176 mg, 1.27 mmol, 3 eq). The mixture was stirred at 60° C. for 12 h. LC-MS showed starting material was consumed completely and desired mass was detected. The reaction mixture was diluted with H₂O (10 mL) and extracted with EtOAc (5 mL*3). The combined organic layers were washed with brine (60 mL), dried over Na₂SO₄, filtered, and concentrated under reduced pressure to give a residue. The residue was purified by column chromatography (SiO₂, Petroleum ether/Ethyl acetate=3/1 to 1/1) to provide product (140 mg, 251 μmol, 59% yield) as a white solid.

LCMS: RT=0.444 min, MS cal.: 557.2, [M+H]⁺=558.3

### Preparation of Intermediate Methyl (S)-2-(1-(4-(6-((4-cyano-2-fluorobenzyl)oxy)pyridin-2-yl)piperidin-1-yl)ethyl)-4-methoxy-1-methyl-1H-benzo[d]imidazole-6-carboxylate (3) and Methyl (R)-2-(1-(4-(6-((4-cyano-2-fluorobenzyl)oxy)pyridin-2-yl)piperidin-1-yl)ethyl)-4-methoxy-1-methyl-1H-benzo[d]imidazole-6-carboxylate (4)

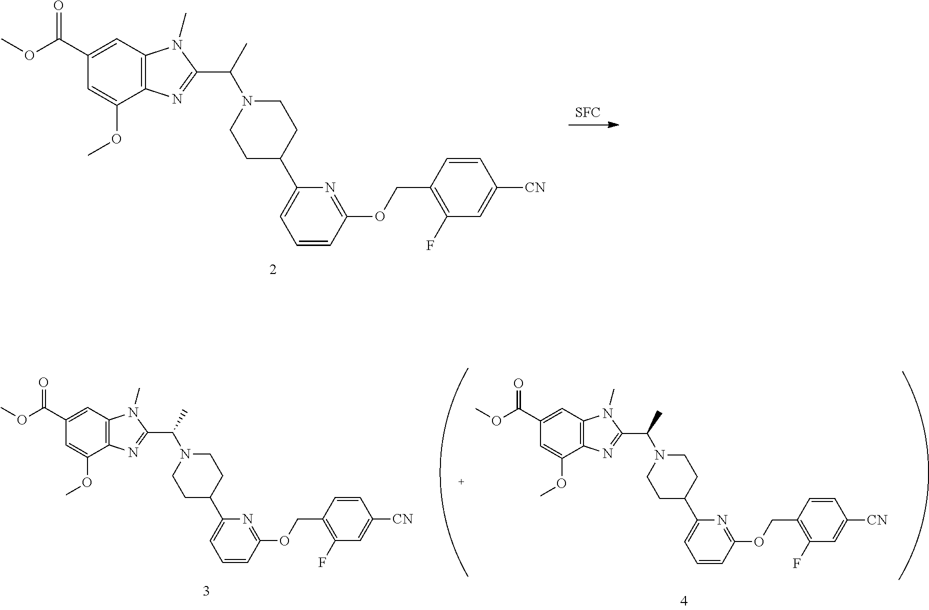

Methyl 2-(1-(4-(6-((4-cyano-2-fluorobenzyl)oxy)pyridin-2-yl)piperidin-1-yl)ethyl)-4-methoxy-1-methyl-1H-benzo[d]imidazole-6-carboxylate 2 (140 mg, 251 μmol, 1 eq) was separated by SFC (column: DAICEL CHIRALCEL OD (250 mm*30 mm, 10 um); mobile phase: [CO₂-MeOH (0.1% NH₃H₂O)]; B %: 50%, isocratic elution mode) to provide 3 (70 mg, 126 μmol, 50% yield) as a yellow oil and 4 (70 mg, 126 μmol, 50% yield) as a yellow oil.

LCMS compound 3: RT=0.434 min, MS cal.: 557.2, [M+H]⁺=558.3

LCMS compound 4: RT=0.433 min, MS cal.: 557.2, [M+H]⁺=558.3

¹H NMR (400 MHz, DMSO-d₆) δ=7.90-7.80 (m, 2H), 7.72-7.55 (m, 3H), 7.26 (s, 1H), 6.84 (d, J=7.2 Hz, 1H), 6.69 (d, J=8.0 Hz, 1H), 5.50-5.37 (s, 2H), 4.29 (q, J=6.4 Hz, 1H), 3.98 (s, 3H), 3.93 (s, 3H), 3.85 (s, 3H), 3.02-2.98 (m, 1H), 2.58-2.51 (m, 3H), 2.23-2.21 (m, 1H), 1.85-1.62 (m, 3H), 1.51-1.39 (m, 4H)

¹H NMR (400 MHz, DMSO-d₆) δ=7.90-7.81 (m, 2H), 7.73-7.56 (m, 3H), 7.26 (s, 1H), 6.84 (d, J=7.2 Hz, 1H), 6.69 (d, J=8.4 Hz, 1H), 5.45 (s, 2H), 4.34 (d, J=4.4 Hz, 1H), 4.00 (s, 3H), 3.92 (s, 3H), 3.87 (s, 3H), 2.98-2.96 (m, 1H), 2.62-2.53 (m, 3H), 2.23-2.21 (m, 1H), 1.83-1.60 (m, 3H), 1.53-1.38 (m, 4H)

### Preparation of (S)-2-(1-(4-(6-((4-Cyano-2-fluorobenzyl)oxy)pyridin-2-yl)piperidin-1-yl)ethyl)-4-methoxy-1-methyl-1H-benzo[d]imidazole-6-carboxylic acid Compound 102 (5)

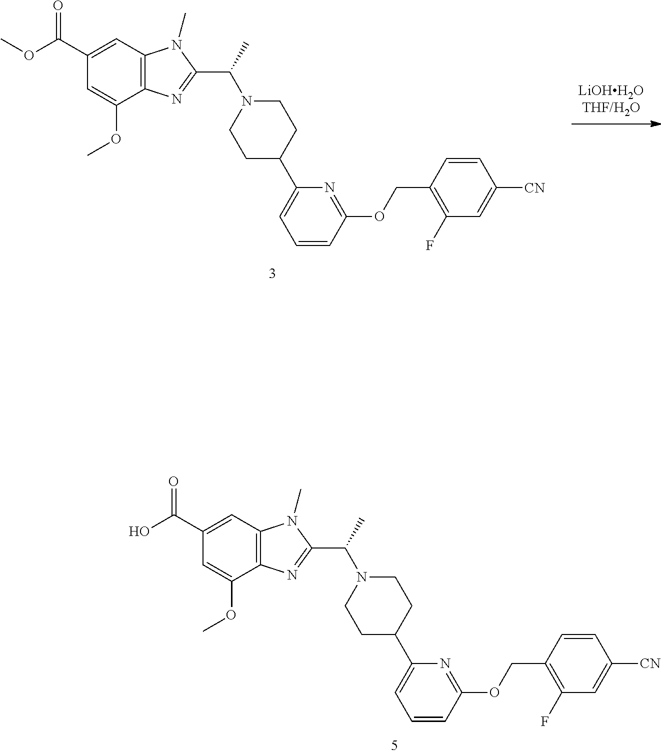 [SMILES: COC(=O)c1cc(OC)c2nc([C@H](C)N3CCC(c4cccc(OCc5ccc(C#N)cc5F)n4)CC3)n(C)c2c1 ; COc1cc(C(=O)O)cc2c1nc([C@H](C)N1CCC(c3cccc(OCc4ccc(C#N)cc4F)n3)CC1)n2C]

A mixture of methyl (S)-2-(1-(4-(6-((4-cyano-2-fluorobenzyl)oxy)pyridin-2-yl)piperidin-1-yl)ethyl)-4-methoxy-1-methyl-1H-benzo[d]imidazole-6-carboxylate 3 (60 mg, 108 μmol, 1 eq), LiOH·H₂O (6.77 mg, 161 μmol, 1.5 eq) in THF (0.7 mL) and H₂O (0.3 mL) was degassed and purged with N₂ for 3 times, and then the mixture was stirred at 25° C. for 12 h under N₂ atmosphere. LCMS showed the starting material was consumed completely. The residue was purified by prep-HPLC (neutral condition; column: Waters Xbridge Prep OBD C18 150*40 mm*10 um; mobile phase: [H₂O (0.05% NH₃H₂O+10 mM NH₄HCO₃)-ACN]; gradient: 30%-60% B over 8.0 min) to provide product (29.7 mg, 54.7 μmol, 50% yield) as a white solid.

LCMS: RT=2.584 min, MS cal.: 543.2, [M+H]⁺=544.2

HPLC: RT=9.685 min, purity: 99.23%

¹H NMR (400 MHz, METHANOL-d₄) δ=7.86 (d, J=1.2 Hz, 1H), 7.65 (t, J=7.6 Hz, 1H), 7.60-7.56 (m, 1H), 7.55-7.49 (m, 2H), 7.45 (d, J=1.2 Hz, 1H), 6.81 (d, J=7.2 Hz, 1H), 6.67 (d, J=8.0 Hz, 1H), 5.50 (s, 2H), 4.35-4.24 (m, 1H), 4.04 (s, 3H), 4.00 (s, 3H), 3.06-2.91 (m, 2H), 2.67-2.50 (m, 2H), 2.41-2.30 (m, 1H), 1.92-1.66 (m, 4H), 1.60 (d, J=6.8 Hz, 3H)

### Example A44. —Preparation of Compound 103. (R)-2-(1-(4-(6-((4-Cyano-2-fluorobenzyl)oxy)pyridin-2-yl)piperidin-1-yl)ethyl)-4-methoxy-1-methyl-1H-benzo[d]imidazole-6-carboxylic acid (Compound 103) (2)

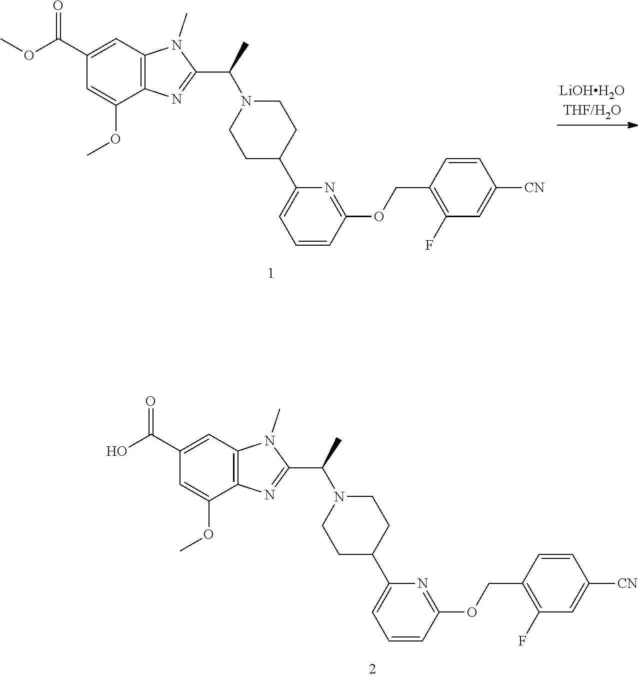 [SMILES: COC(=O)c1cc(OC)c2nc([C@@H](C)N3CCC(c4cccc(OCc5ccc(C#N)cc5F)n4)CC3)n(C)c2c1 ; COc1cc(C(=O)O)cc2c1nc([C@@H](C)N1CCC(c3cccc(OCc4ccc(C#N)cc4F)n3)CC1)n2C]

A mixture of methyl (R)-2-(1-(4-(6-((4-cyano-2-fluorobenzyl)oxy)pyridin-2-yl)piperidin-1-yl)ethyl)-4-methoxy-1-methyl-1H-benzo[d]imidazole-6-carboxylate 1 (60 mg, 108 μmol, 1 eq), LiOH·H₂O (6.77 mg, 161 μmol, 1.5 eq) in THF (0.7 mL) and H₂O (0.3 mL) was degassed and purged with N₂ for 3 times, and then the mixture was stirred at 25° C. for 12 h under N₂ atmosphere. LCMS showed the starting material was consumed completely. The residue was purified by prep-HPLC (neutral condition column: Waters Xbridge Prep OBD C18 150*40 mm*10 um; mobile phase: [H₂O (10 mM NH₄HCO₃)-ACN]; gradient: 15%-55% B over 8.0 min) to provide product (27.9 mg, 51.3 μmol, 47% yield) as a white solid.

LCMS: RT=2.186 min, MS cal.: 543.2, [M+H]⁺=544.2

HPLC: RT=9.671 min, purity: 100%

1H NMR (400 MHz, METHANOL-d4) δ=7.86 (d, J=1.2 Hz, 1H) 7.65 (t, J=7.6 Hz, 1H) 7.60-7.49 (m, 3H) 7.45 (d, J=0.8 Hz, 1H) 6.81 (d, J=7.2 Hz, 1H) 6.67 (d, J=8.0 Hz, 1H) 5.49 (s, 2H) 4.30 (q, J=6.8 Hz, 1H) 4.04 (s, 3H) 4.00 (s, 3H) 3.09-2.89 (m, 2H) 2.66-2.51 (m, 2H) 2.42-2.28 (m, 1H) 1.94-1.67 (m, 4H) 1.60 (d, J=6.8 Hz, 3H)

### Example A45-Preparation of Compound 310. (R)-2-((4-(3-(4-cyano-2-fluorophenyl)-2,3-dihydrobenzo[b][1,4]dioxin-5-yl)piperidin-1-yl)methyl)-4-methoxy-1-methyl-1H-benzo[d]imidazole-6-carboxylic acid (Compound 310)

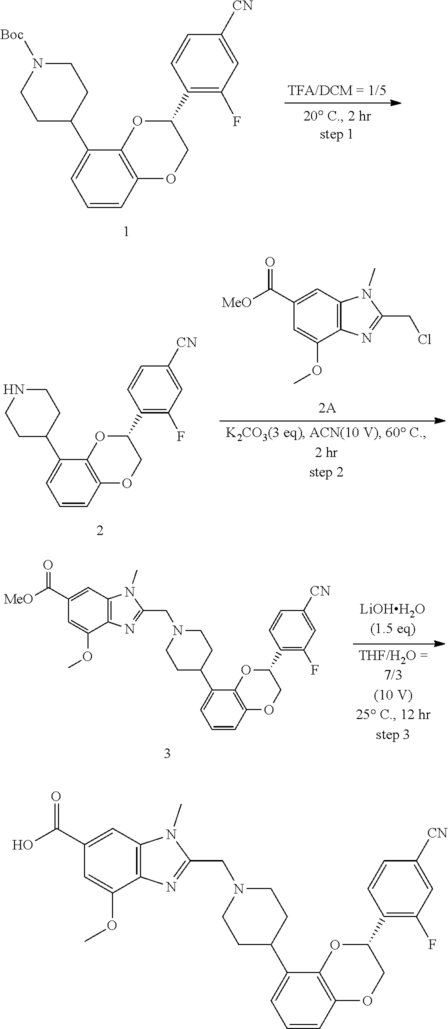 [SMILES: CC(C)(C)OC(=O)N1CCC(c2cccc3c2O[C@H](c2ccc(C#N)cc2F)CO3)CC1 ; N#Cc1ccc([C@@H]2COc3cccc(C4CCNCC4)c3O2)c(F)c1 ; COC(=O)c1cc(OC)c2nc(CCl)n(C)c2c1 ; COC(=O)c1cc(OC)c2nc(CN3CCC(c4cccc5c4O[C@H](c4ccc(C#N)cc4F)CO5)CC3)n(C)c2c1 ; COc1cc(C(=O)O)cc2c1nc(CN1CCC(c3cccc4c3O[C@H](c3ccc(C#N)cc3F)CO4)CC1)n2C]

### General Procedure for Preparation of Intermediate 2:

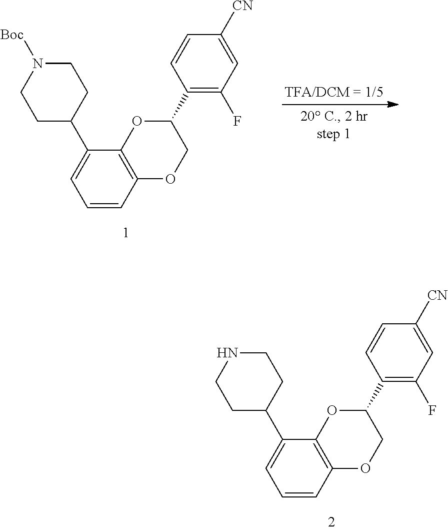 [SMILES: CC(C)(C)OC(=O)N1CCC(c2cccc3c2O[C@H](c2ccc(C#N)cc2F)CO3)CC1 ; N#Cc1ccc([C@@H]2COc3cccc(C4CCNCC4)c3O2)c(F)c1]

To a solution of 1 (65 mg, 148.24 μmol, 1 eq) in DCM (1 mL) was added TFA (0.2 mL). The mixture was stirred at 20° C. for 2 hr. LCMS (RT=1.223 min) showed the starting material was consumed completely. The reaction mixture was concentrated under reduced pressure to give a residue. The crude product 2 (67 mg, crude, TFA) was used into the next step without further purification.

LCMS: RT=1.223 min, MS cal.: 338.4, [M+1]⁺=339.1

### General Procedure for Preparation of Intermediate 3:

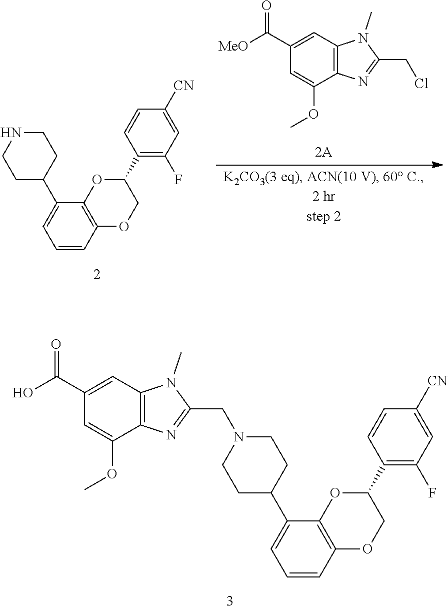 [SMILES: N#Cc1ccc([C@@H]2COc3cccc(C4CCNCC4)c3O2)c(F)c1 ; COC(=O)c1cc(OC)c2nc(CCl)n(C)c2c1 ; COc1cc(C(=O)O)cc2c1nc(CN1CCC(c3cccc4c3O[C@H](c3ccc(C#N)cc3F)CO4)CC1)n2C]

A mixture of 2A (39.79 mg, 148.10 μmol, 1 eq), 2 (67 mg, 148.10 μmol, 1 eq, TFA), K₂CO₃ (61.40 mg, 444.30 μmol, 3 eq) in ACN (1 mL) was degassed and purged with N₂ for 3 times, and then the mixture was stirred at 60° C. for 2 hr under N₂ atmosphere. LCMS (RT=1.520 min) showed the starting material was consumed completely. The reaction mixture was diluted with H₂O (10 mL) and extracted with EtOAc (15 mL*3). The combined organic layers were washed with brine (10 mL*2), dried over Na₂SO₄, filtered and concentrated under reduced pressure to give a residue. The residue was purified by column chromatography (SiO₂, Petroleum ether/Ethyl acetate=100/1 to 0/1). 3 (70 mg, 122.68 μmol, 82.83% yield) was obtained as a white solid.

LCMS: RT=1.521 min, MS cal.: 570.6, [M+H]⁺=571.1

¹H NMR (400 MHz, CHCl3-d) δ=7.78 (s, 1H), 7.65-7.55 (m, 2H), 7.45 (d, J=9.6 Hz, 1H), 7.39 (s, 1H), 6.91-6.79 (m, 3H), 5.52-5.48 (m, 1H), 4.46 (dd, J=2.4, 11.2 Hz, 1H), 4.07 (s, 3H), 4.00 (s, 1H), 3.93-3.92 (m, 1H), 4.01-3.92 (m, 5H), 3.87 (s, 2H), 3.03-2.91 (m, 3H), 2.36-2.19 (m, 2H), 1.94-1.66 (m, 4H)

### General Procedure for Preparation of Compound 310:

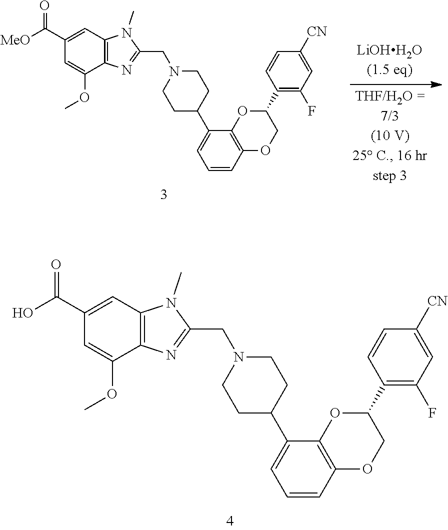 [SMILES: COC(=O)c1cc(OC)c2nc(CN3CCC(c4cccc5c4O[C@H](c4ccc(C#N)cc4F)CO5)CC3)n(C)c2c1 ; COc1cc(C(=O)O)cc2c1nc(CN1CCC(c3cccc4c3O[C@H](c3ccc(C#N)cc3F)CO4)CC1)n2C]

To a solution of 3 (60 mg, 105.15 μmol, 1 eq) in THF (1.4 mL) was added LiOH·H₂O (6.62 mg, 157.73 μmol, 1.5 eq) in H₂O (0.6 mL). The mixture was stirred at 25° C. for 12 hr. LCMS showed ˜ 47% of reactant 1 remained. Then LiOH·H₂O (2.21 mg, 52.58 μmol, 0.5 eq) in H₂O (0.3 mL) was added to the mixture and stirred at 25° C. for 4 hr. LCMS (ET53089-1171-P1R, product: RT=1.050 min) showed the starting material was consumed completely. The crude product was purified by reversed-phase HPLC (column: Waters Xbridge BEH C18 100*30 mm*10 um; mobile phase: [H₂O (10 mM NH₄HCO₃)-ACN]; gradient: 25%-60% B over 8.0 min). (R)-2-((4-(3-(4-cyano-2-fluorophenyl)-2,3-dihydrobenzo[b][1,4]dioxin-5-yl)piperidin-1-yl)methyl)-4-methoxy-1-methyl-1H-benzo[d]imidazole-6-carboxylic acid_(23.89 mg, 42.92 μmol, 40.82% yield) was obtained as a white solid.

LCMS: RT=2.568 min, MS cal.: 556.6, [M+H]⁺=557.3

HPLC: RT=10.255 min, purity=99.241%

SFC: RT=3.347 min, ee %=96.42%

¹H NMR (400 MHz, DMSO-d₆) δ=7.99 (dd, J=1.6, 10.3 Hz, 1H), 7.84 (dd, J=1.2, 8.0 Hz, 1H), 7.74 (s, 1H), 7.67 (t, J=7.6 Hz, 1H), 7.26 (d, J=1.2 Hz, 1H), 6.87-6.80 (m, 2H), 6.79-6.74 (m, 1H), 5.58 (dd, J=2.0, 7.4 Hz, 1H), 4.47 (dd, J=2.4, 11.2 Hz, 1H), 4.10 (dd, J=7.6, 11.4 Hz, 1H), 3.93 (s, 3H), 3.86 (s, 3H), 3.82-3.72 (m, 2H), 2.97-2.79 (m, 3H), 2.21-2.08 (m, 2H), 1.84-1.54 (m, 4H)

### Example A46—Preparation of Compound 311. 2-((4-(6-((4-chloro-2-fluorobenzyl)oxy)pyridin-2-yl)piperidin-1-yl)methyl)-3-methyl-7,8-dihydro-3H-[1,4]dioxino[2′,3′:3,4]benzo[1,2-d]imidazole-5-carboxylic acid (Compound 311) (11)

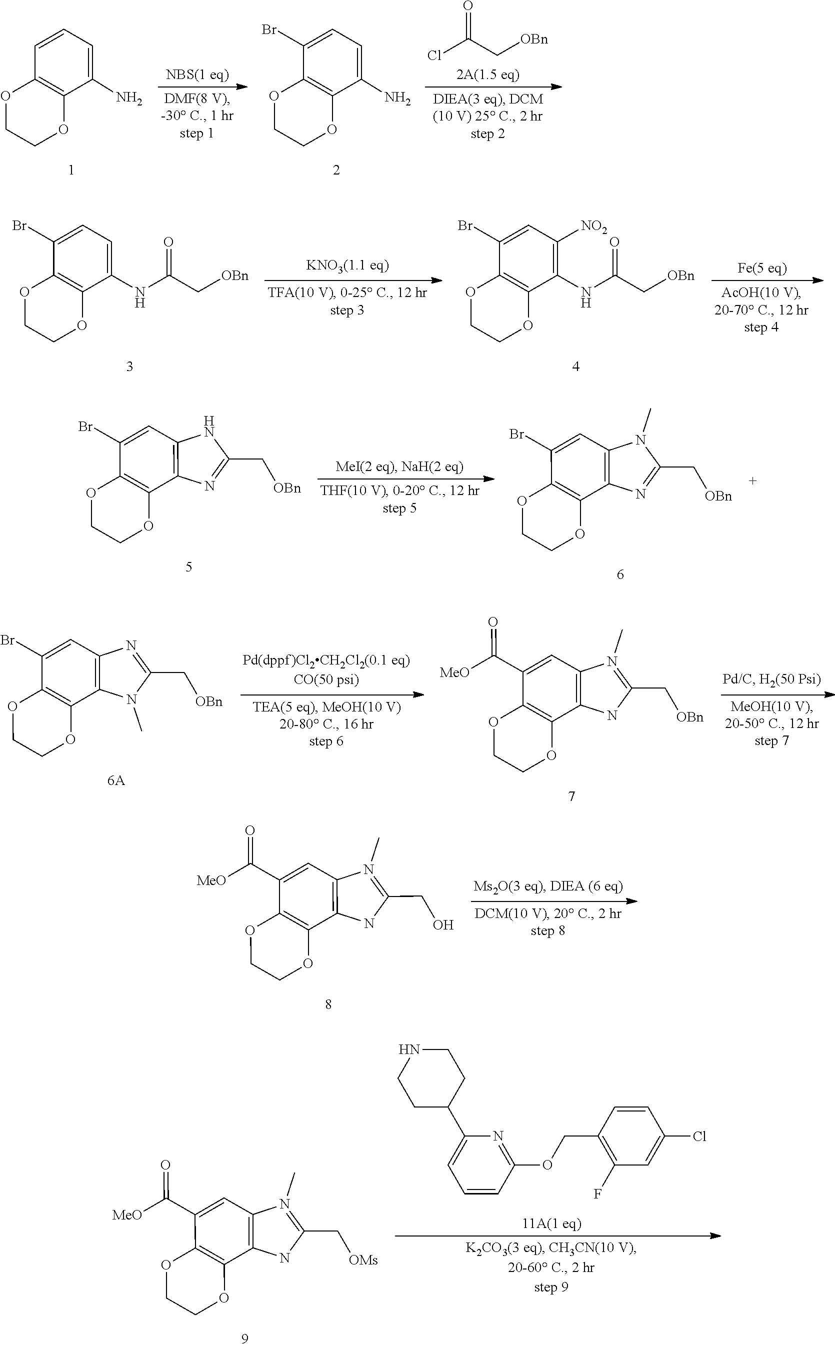

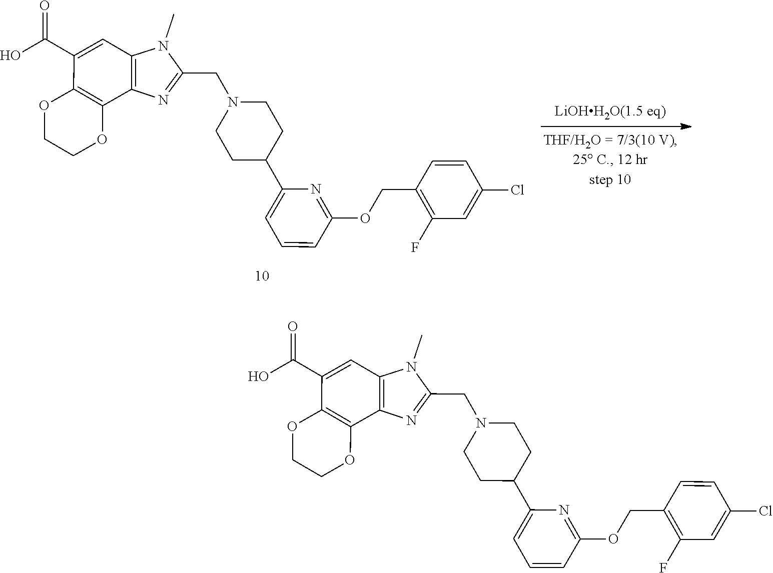 [SMILES: Cn1c(CN2CCC(c3cccc(OCc4ccc(Cl)cc4F)n3)CC2)nc2c3c(c(C(=O)O)cc21)OCCO3 ; Cn1c(CN2CCC(c3cccc(OCc4ccc(Cl)cc4F)n3)CC2)nc2c3c(c(C(=O)O)cc21)OCCO3]

### General Procedure for Preparation of Intermediate 2:

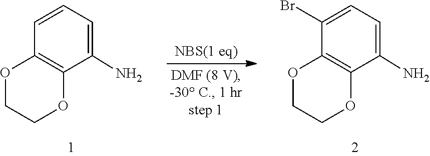 [SMILES: Nc1cccc2c1OCCO2 ; Nc1ccc(Br)c2c1OCCO2]

The reaction was set up in 2 batches.

A mixture of 1 (5 g, 33.08 mmol, 1 eq) in DMF (20 mL) was degassed and purged with N₂ for 3 times, then a solution of NBS (5.89 g, 33.08 mmol, 1 eq) in DMF (20 mL) was added dropwise to the mixture at −30° C. The mixture was stirred at −30° C. for 1 hr under N₂ atmosphere. TLC (PE/EtOAc=2/1, product R_{f}=0.47) showed the 1 was consumed completely. Two batches were combined to work up. The mixture was poured into water 100 mL at 0° C. Solid was precipitate out. Then filtered and the filtered cake was co-evaporated with ACN 50 mL. The operation was repeated for twice. Compound 2 (12 g, 52.16 mmol, 78.85% yield) was obtained as a pink solid.

LCMS: RT=0.999 min, MS cal.: 229.0/231.0, [M+H]⁺=230.0/232.0

¹H NMR (400 MHz, CHCl3-d) δ=6.89 (d, J=8.4 Hz, 1H), 6.25 (d, J=8.4 Hz, 1H), 4.39-4.33 (m, 2H), 4.33-4.27 (m, 2H), 3.74 (br s, 2H)

### General Procedure for Preparation of Intermediate 3:

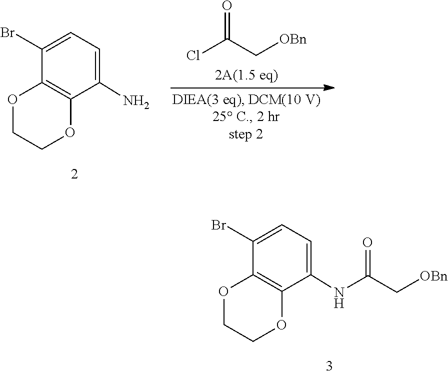 [SMILES: Nc1ccc(Br)c2c1OCCO2 ; O=C(Cl)COCc1ccccc1 ; O=C(COCc1ccccc1)Nc1ccc(Br)c2c1OCCO2]

The reaction was set up in 2 batches.

A mixture of 2 (6 g, 26.08 mmol, 1 eq), 2A (7.22 g, 39.12 mmol, 6.07 mL, 1.5 eq), DIEA (10.11 g, 78.24 mmol, 13.63 mL, 3 eq) in DCM (60 mL) was degassed and purged with N₂ for 3 times, and then the mixture was stirred at 25° C. for 2 hr under N₂ atmosphere. TLC (PE/EtOAc=2/1, product R_{f}=0.43) indicated 2 was consumed completely and one new spot formed. Two batches were combined to work up. The reaction mixture was quenched by addition H₂O 60 mL at 20° C., and extracted with DCM 40 mL*3. The combined organic layers were washed with brine 30 mL*5, dried over Na₂SO₄, filtered and concentrated under reduced pressure to give a residue. The residue was purified by column chromatography (SiO₂, Petroleum ether/Ethyl acetate=5/1 to 0/1). 3 (16 g, 42.30 mmol, 81.10% yield) was obtained as a white solid.

LCMS: RT=1.380 min, MS cal.: 377.0/379.0, [M+H]⁺=378.0/380.0

¹H NMR (400 MHz, CHCl3-d) δ=8.83 (br s, 1H), 7.89 (d, J=9.2 Hz, 1H), 7.45-7.32 (m, 5H), 7.10 (d, J=9.2 Hz, 1H), 4.68 (s, 2H), 4.42-4.36 (m, 2H), 4.35-4.31 (m, 2H), 4.12 (s, 2H)

### General Procedure for Preparation of Intermediate 4:

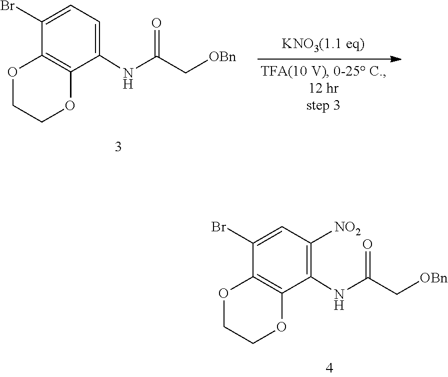 [SMILES: O=C(COCc1ccccc1)Nc1ccc(Br)c2c1OCCO2 ; O=C(COCc1ccccc1)Nc1c([N+](=O)[O-])cc(Br)c2c1OCCO2]

TFA (160 mL) was charged to a three-necked round bottom flask, then 3 (16 g, 42.30 mmol, 1 eq) was added to the mixture at 0° C. At 0° C., KNO₃ (4.70 g, 46.53 mmol, 1.1 eq) was added in portions to the reaction mixture at 0° C. After the addition, the mixture was stirred at 25° C. for 12 hr. TLC (PE/EtOAc=2/1, product R_{f}=0.35, byproduct R_{f}=0.21) showed the 3 was consumed completely. The reaction mixture was added dropwise to H₂O 150 mL at 0° C., then extracted with DCM 50 mL*4. The combined organic layers were added sat. Na₂CO₃ to adjust pH=8-9 at 0° C. and was poured into separatory funnel and separated. The organic layer was washed with brine 50 mL*2, dried over Na₂SO₄, filtered and the filtrate was concentrated to give a crude product. The residue was purified by column chromatography (SiO₂, Petroleum ether/Ethyl acetate=2/1 to 0/1). 4 (7.8 g, 18.43 mmol, 43.57% yield) was obtained as a white solid.

LCMS: RT=1.331 min, MS cal.: 422.0/424.0, [M+H]⁺=423.0/425.0

¹H NMR (400 MHz, CHCl3-d) δ=8.95 (s, 1H), 7.92 (s, 1H), 7.46-7.33 (m, 5H), 4.71 (s, 2H), 4.48 (dd, J=3.2, 5.2 Hz, 2H), 4.36 (dd, J=3.2, 5.2 Hz, 2H), 4.16 (s, 2H)

### General Procedure for Preparation of Intermediate 5:

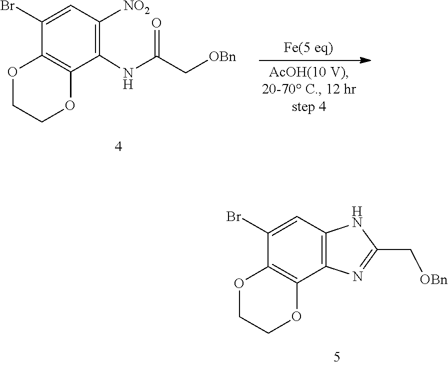 [SMILES: O=C(COCc1ccccc1)Nc1c([N+](=O)[O-])cc(Br)c2c1OCCO2 ; Brc1cc2[nH]c(COCc3ccccc3)nc2c2c1OCCO2]

The reaction was set up in 2 batches.

AcOH (45 mL) was charged to a 100 mL three-necked round bottom flask, then 4 (4.5 g, 10.63 mmol, 1 eq) was added to the mixture at 20° C. The mixture was heated to 50° C. At 50° C., Fe (2.97 g, 53.16 mmol, 5 eq) was added in portions to the reaction mixture at 50° C. After the addition, the mixture was stirred at 70° C. for 12 hr. LCMS (ET64759-704-P1A1, product: RT=1.147 min) showed the 4 was consumed completely, one main peak with desired mass was detected. The reaction mixture was cooled down to 30° C., then filtered through a pad of Celite, the filter cake was washed with EtOAc 30 mL*3. The combined organic layers were concentrated to give a residue. The residue was dissolved with EtOAc 60 mL, then washed with H₂O 20 mL, sat. Na₂CO₃ 20 mL*2 (until the aqueous layer's pH=9˜10), brine 20 mL*2, dried over Na₂SO₄, filtered, and concentrated under reduced pressure to give a residue. Compound 5 (7.22 g, 19.24 mmol, 90.48% yield) was obtained as a brown solid.

LCMS: RT=1.331 min, MS cal.: 374.0/376.0, [M+H]⁺=375.0/377.0

¹H NMR (400 MHz, DMSO-d6) δ=7.40-7.26 (m, 6H), 4.65 (s, 2H), 4.57 (s, 2H), 4.37 (s, 4H)

### General Procedure for Preparation of Intermediate 6 & Intermediate 6A:

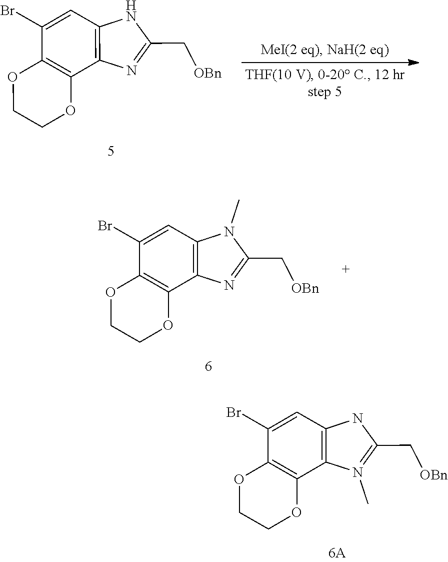

To a solution of 5 (6 g, 15.99 mmol, 1 eq) in THF (60 mL) was added NaH (1.28 g, 31.98 mmol, 60% purity, 2 eq) in portions at 0° C. under N₂. The mixture was stirred at 0° C. for 0.5 hr. At 0° C., Mel (4.54 g, 31.98 mmol, 1.99 mL, 2 eq) was added in portions to the reaction mixture. After the addition, the mixture was stirred at 20° C. for 11.5 hr. TLC (PE/EtOAc=1/1, 6 R_{f}=0.17, 6A R_{f}=0.34) showed the 5 was consumed completely. The reaction mixture was quenched by addition H₂O 60 mL at 0° C. and extracted with EtOAc 50 mL*3. The combined organic layers were washed with brine 20 mL*2, dried over Na₂SO₄, filtered, and concentrated under reduced pressure to give a residue. The residue was purified by column chromatography (SiO₂, Petroleum ether/Ethyl acetate=5/1 to 0/1). 6 (3 g, 7.71 mmol, 48.20% yield) was obtained as a white solid. 6A (2 g, 5.14 mmol, 32.13% yield) was obtained as a white solid.

¹H NMR (400 MHz, CHCl3-d) δ=7.37-7.28 (m, 5H), 7.13 (s, 1H), 4.84 (s, 2H), 4.55 (s, 2H), 4.48-4.40 (m, 4H), 3.76 (s, 3H)

¹H NMR (400 MHz, CHCl3-d) δ=7.51 (s, 1H), 7.38-7.29 (m, 5H), 4.77 (s, 2H), 4.56 (s, 2H), 4.45-4.40 (m, 2H), 4.39-4.35 (m, 2H), 4.02 (s, 3H)

### General Procedure for Preparation of Intermediate 7:

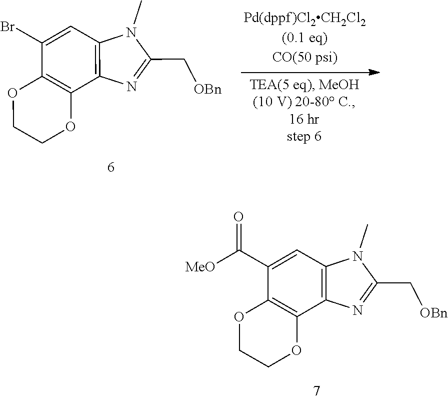 [SMILES: Cn1c(COCc2ccccc2)nc2c3c(c(Br)cc21)OCCO3 ; COC(=O)c1cc2c(nc(COCc3ccccc3)n2C)c2c1OCCO2]

MeOH (30 mL) was charged to a hydrogenating flask, 6 (3 g, 7.71 mmol, 1 eq) and TEA (3.90 g, 38.54 mmol, 5.36 mL, 5 eq) was added to the solution at 20° C. At 20° C., Pd(dppf Cl₂·CH₂Cl₂ (629.41 mg, 770.73 μmol, 0.1 eq) was added to the reaction mixture under Ar atmosphere. The mixture was degassed under vacuum and purged with CO 3 times. The mixture was stirred under CO (50 psi) at 80° C. for 16 hours. LCMS (ET64759-717-P1A, product: RT=1.121 min) showed the starting material was consumed completely, one main peak with desired mass was detected. The reaction mixture was cooled down 20° C. and filtered, the filter cake was washed EtOAc 10 mL*3. The filtrate was added H₂O 10 mL and FA (1 M) to adjust pH=6-7 and extracted with EtOAc 30 mL*3. The combined organic layers were washed with brine 20 mL*2, dried over Na₂SO₄, filtered and concentrated under reduced pressure to give a residue. The residue was purified by column chromatography (SiO₂, Petroleum ether/Ethyl acetate=1/1 to 0/1). 7 (2.3 g, 6.24 mmol, 81.01% yield) was obtained as a red oil.

LCMS: RT=1.181 min, MS cal.: 368.1, [M+H]⁺=369.1

¹H NMR (400 MHz, CHCl3-d) δ=7.49 (s, 1H), 7.37-7.28 (m, 5H), 4.87 (s, 2H), 4.57 (s, 2H), 4.51-4.47 (m, 2H), 4.45-4.41 (m, 2H), 3.94 (s, 3H), 3.82 (s, 3H)

### General Procedure for Preparation of Intermediate 8:

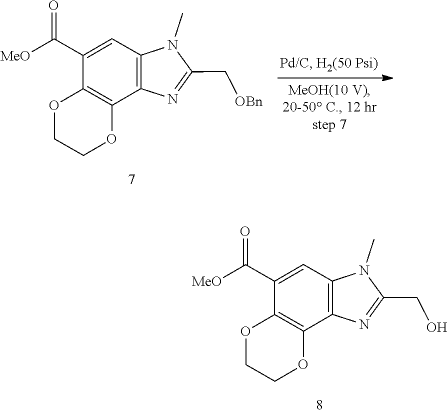 [SMILES: COC(=O)c1cc2c(nc(COCc3ccccc3)n2C)c2c1OCCO2 ; COC(=O)c1cc2c(nc(CO)n2C)c2c1OCCO2]

Pd/C (300 mg) was added to hydrogenation bottle under Ar, then MeOH (1.5 mL) was added. 7 (300 mg, 814.37 μmol, 1 eq) in MeOH (1.5 mL) was added to solution A at 20° C. under Ar atmosphere. The mixture was degassed under vacuum and purged with H2 3 times. The mixture was stirred under H₂ (50 psi) at 50° C. for 12 hours. LCMS (ET64759-719-P1A1, product: RT=0.218 min) showed the 7 was consumed completely, one main peak with desired mass was detected. The suspension was filtered through a pad of Celite, and the cake was washed with MeOH 20 mL×5. The combined filtrates were concentrated to give a product. 8 (220 mg, 790.63 μmol, 97.08% yield) was obtained as a colorless oil.

LCMS: RT=0.240 min, MS cal.: 278.1, [M+H]⁺=279.1

¹H NMR (400 MHz, CHCl3-d) δ=7.47 (s, 1H), 4.90 (s, 2H), 4.48 (dd, J=2.8, 5.2 Hz, 2H), 4.45-4.39 (m, 2H), 3.93 (s, 3H), 3.80 (s, 3H), 3.49 (s, 1H)

### General Procedure for Preparation of Intermediate 9:

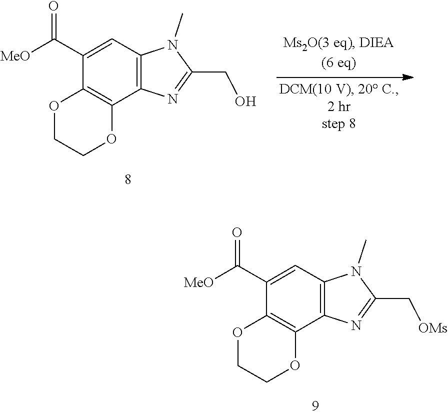 [SMILES: COC(=O)c1cc2c(nc(CO)n2C)c2c1OCCO2 ; COC(=O)c1cc2c(nc(COS(C)(=O)=O)n2C)c2c1OCCO2]

To a solution of 8 (200 mg, 718.75 μmol, 1 eq) and DIEA (557.36 mg, 4.31 mmol, 751.16 μL, 6 eq) in DCM (2 mL) was added Ms₂O (375.61 mg, 2.16 mmol, 3 eq) at 20° C. The mixture was stirred at 20° C. for 2 hr. LCMS (product: RT=0.353 min) showed the 8 was consumed completely. The reaction mixture was quenched by addition H₂O 5 mL at 20° C. and extracted with DCM 5 mL*3. The combined organic layers were washed with FA (1 M) to adjust pH=5-6, then the organic layer was washed with sat. Na₂CO₃ to adjust pH=8-9, and washed with brine 5 mL*2, dried over Na₂SO₄, filtered and concentrated under reduced pressure to give a residue. 9 (200 mg, crude) was obtained as a yellow oil.

### General Procedure for Preparation of Intermediate 10:

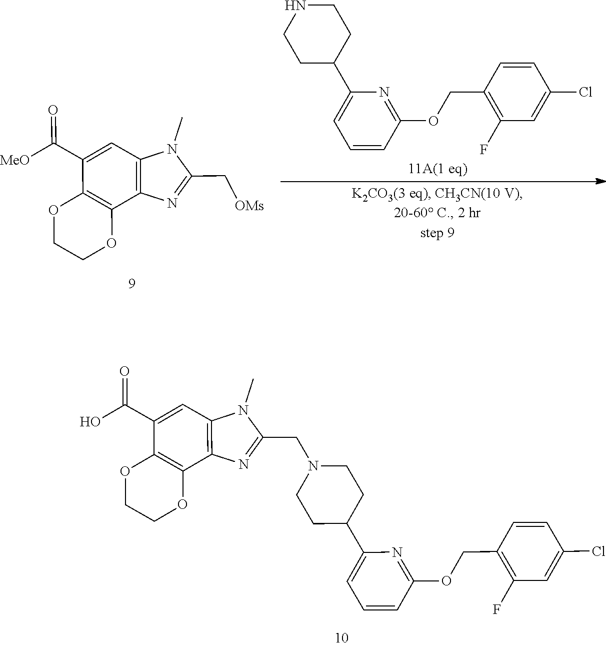 [SMILES: COC(=O)c1cc2c(nc(COS(C)(=O)=O)n2C)c2c1OCCO2 ; Fc1cc(Cl)ccc1COc1cccc(C2CCNCC2)n1 ; Cn1c(CN2CCC(c3cccc(OCc4ccc(Cl)cc4F)n3)CC2)nc2c3c(c(C(=O)O)cc21)OCCO3]

To a solution of 9 (100 mg, 224.50 μmol, 1 eq) and 11A (72.02 mg, 224.50 μmol, 1 eq) in CH₃CN (1 mL) was added K₂CO₃ (93.08 mg, 673.49 μmol, 3 eq) at 20° C. The mixture was stirred at 60° C. for 2 hr. LCMS (ET64759-731-PIA, product: RT=0.448 min) showed the 9 was consumed completely. The reaction mixture was cooled down to 20° C. and quenched by addition H₂O 3 mL at 20° C., and then extracted with EtOAc 5 mL*3. The combined organic layers were washed with brine 3 mL*2, dried over Na₂SO₄, filtered, and concentrated under reduced pressure to give a residue. The residue was purified by column chromatography (SiO₂, Petroleum ether/Ethyl acetate=1/1 to Ethyl acetate/Methanol=1/1). 10 (50 mg, 86.05 μmol, 38.33% yield) was obtained as a yellow oil.

¹H NMR (400 MHz, CHCl3-d) δ=7.53-7.47 (m, 2H), 7.44 (t, J=8.0 Hz, 1H), 7.14-7.08 (m, 2H), 6.73 (d, J=7.2 Hz, 1H), 6.60 (d, J=8.4 Hz, 1H), 5.41 (s, 2H), 4.48 (dd, J=2.4, 5.2 Hz, 2H), 4.45-4.39 (m, 2H), 3.94 (s, 3H), 3.92 (s, 3H), 3.85 (s, 2H), 2.96 (br d, J=12.0 Hz, 2H), 2.66-2.54 (m, 1H), 2.27 (br t, J=10.4 Hz, 2H), 1.93-1.72 (m, 4H)

### General Procedure for Preparation of Compound 311:

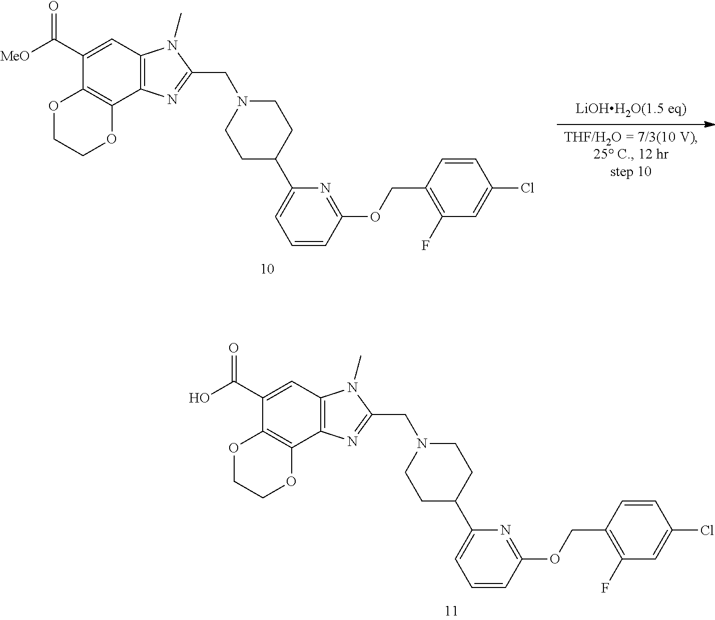

To a solution of 10 (50 mg, 86.05 μmol, 1 eq) in THF (0.35 mL) was added LiOH·H₂O (5.42 mg, 129.08 μmol, 1.5 eq) in H₂O (0.15 mL) at 25° C. The mixture was stirred at 25° C. for 12 hr. LCMS (RT=0.414 min) showed the 10 was consumed completely. The reaction mixture was filtered, then the filtrate was purified by prep-HPLC (column: Waters Xbridge BEH C18 100*30 mm*10 um; mobile phase: [H₂O (10 mM NH₄HCO₃)—CH₃CN]; gradient: 20%-60% B over 8.0 min). 2-((4-(6-((4-Chloro-2-fluorobenzyl)oxy)pyridin-2-yl)piperidin-1-yl)methyl)-3-methyl-7,8-dihydro-3H-[1,4]dioxino[2′,3′:3,4]benzo[1,2-d]imidazole-5-carboxylic acid (27.31 mg, 47.98 μmol, 55.75% yield, 99.61% purity) was obtained as a white solid.

LCMS: RT=2.627 min, MS cal.: 566.2, [M+H]⁺=567.2

HPLC: RT=10.655 min

¹H NMR (400 MHz, MeOH-d4) δ=7.59-7.54 (m, 1H), 7.53 (s, 1H), 7.48 (t, J=8.0 Hz, 1H), 7.23-7.14 (m, 2H), 6.82 (d, J=7.2 Hz, 1H), 6.63 (d, J=8.0 Hz, 1H), 5.40 (s, 2H), 4.43 (dd, J=2.4, 5.2 Hz, 2H), 4.40-4.34 (m, 2H), 3.99-3.88 (m, 5H), 3.08 (d, J=12.0 Hz, 2H), 2.72-2.61 (m, 1H), 2.46-2.33 (m, 2H), 1.95-1.83 (m, 4H)

### Example A47—Preparation of Compound 312 and Compound 313. (R)-2-((4-(6-((4-Cyano-2-fluorobenzyl)oxy)pyridin-2-yl)piperidin-1-yl)methyl)-4-(1-fluoroethoxy)-1-methyl-1H-benzo[d]imidazole-6-carboxylic acid (Compound 312) (4B) and(S)-2-((4-(6-((4-Cyano-2-fluorobenzyl)oxy)pyridin-2-yl)piperidin-1-yl)methyl)-4-(1-fluoroethoxy)-1-methyl-1H-benzo[d]imidazole-6-carboxylic acid (Compound 313) (4A)

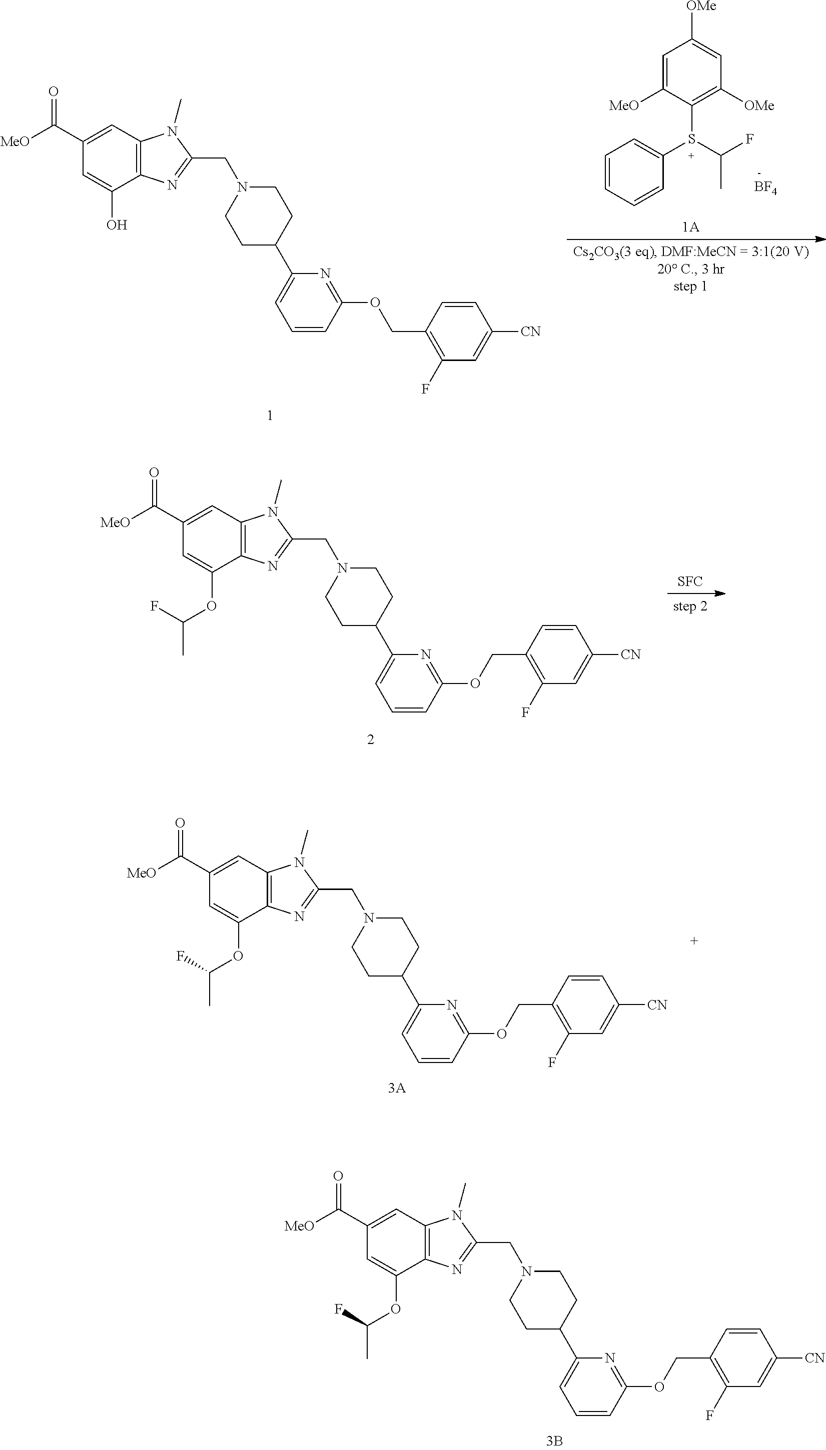

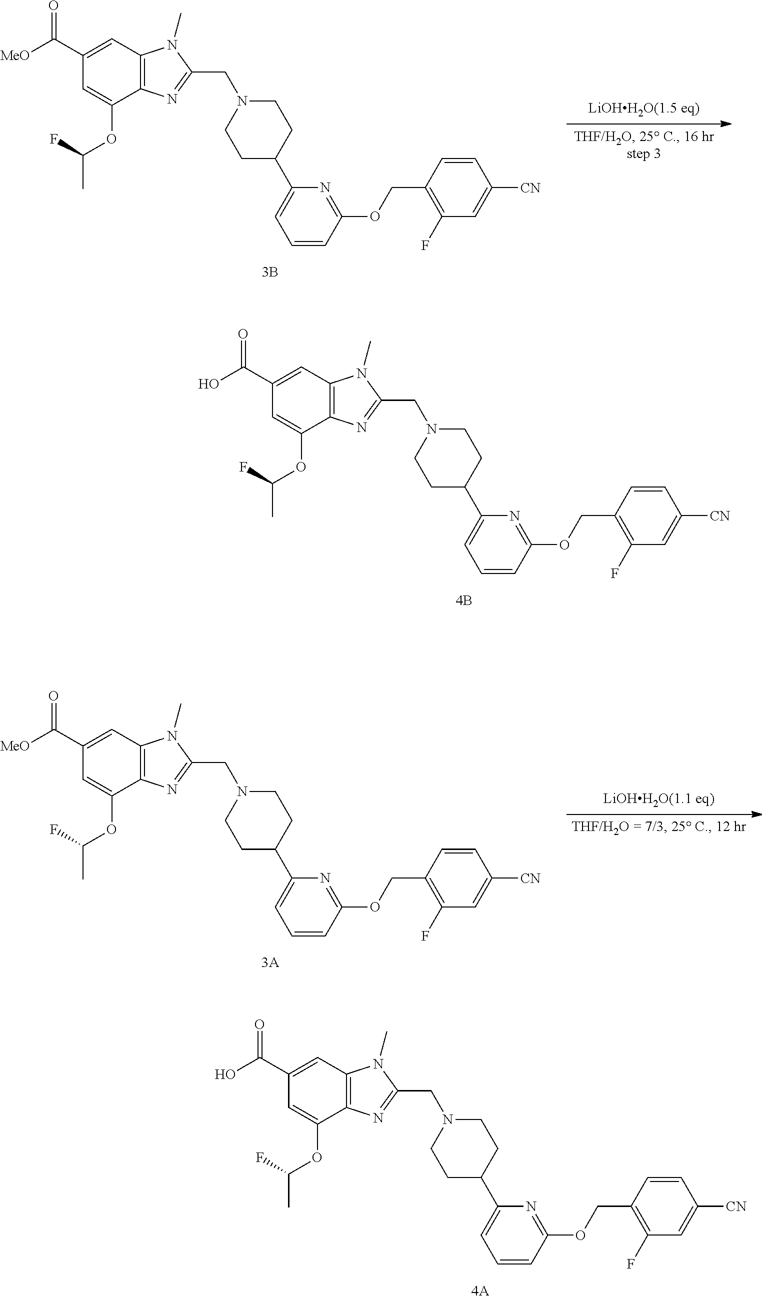

### General Procedure for Preparation of Intermediate 2:

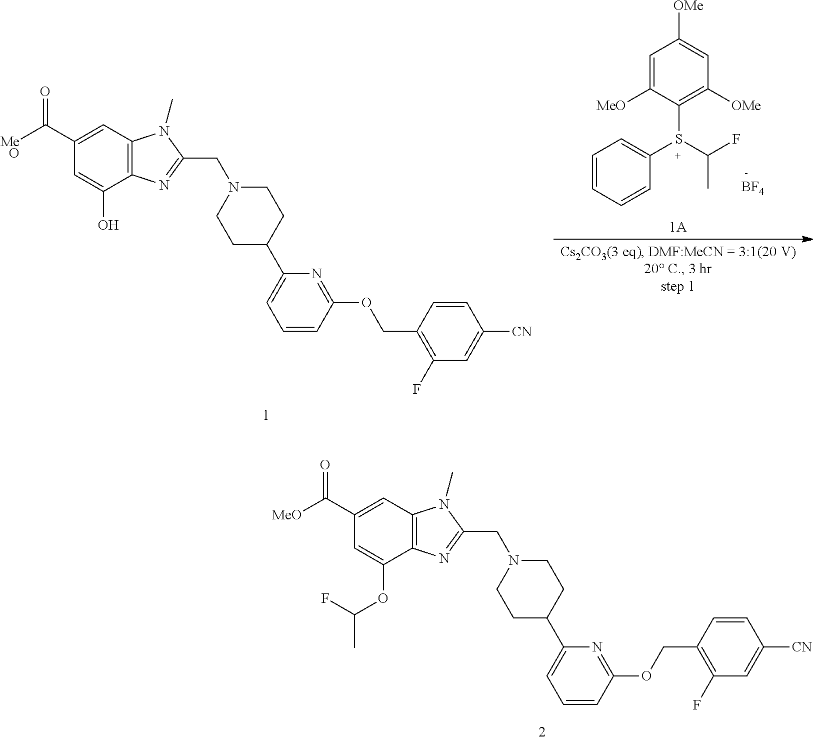

The reactions were carried out in parallel as 3 batches.

To a solution of 1 (100 mg, 188.84 μmol, 1 eq) and Cs₂CO₃ (184.58 mg, 566.51 μmol, 3 eq) in ACN (0.5 mL) and DMF (1.5 mL) was added 1A (122.14 mg, 377.67 μmol, 2 eq). The mixture was stirred at 20° C. for 3 hr. LC-MS showed 1 was consumed completely and one main peak with desired mass was detected. 3 reactions were combined for work-up. The reaction mixture was diluted with H₂O 10 mL and extracted with DCM 30 mL (10 mL*3). The combined organic layers were washed with brine 10 mL, dried over Na₂SO₄, filtered and concentrated under reduced pressure to give a residue. The residue was purified by column chromatography (SiO₂, Petroleum ether/Ethyl acetate=1/0 to 1/1). 2 (295 mg, 512.50 μmol, 90.47% yield) was obtained as a yellow solid.

LCMS: RT=0.456 min, MS cal.: 575.23, [M+H]⁺=576.2

### General Procedure for Preparation of Intermediate 3A & Intermediate 3B:

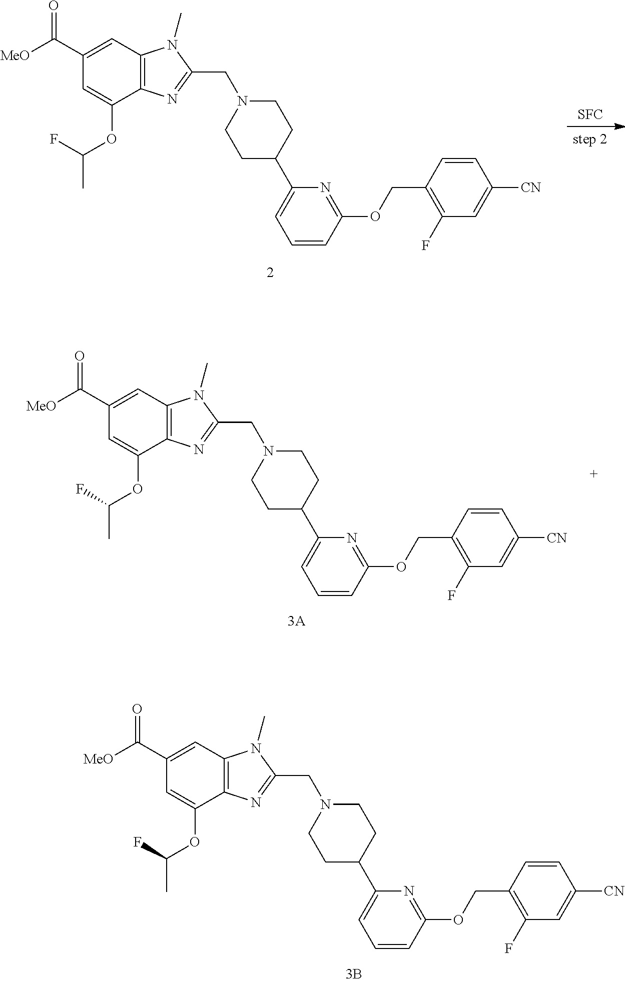 [SMILES: COC(=O)c1cc(OC(C)F)c2nc(CN3CCC(c4cccc(OCc5ccc(C#N)cc5F)n4)CC3)n(C)c2c1 ; "SFC" ; "step 2" ; COC(=O)c1cc(O[C@H](C)F)c2nc(CN3CCC(c4cccc(OCc5ccc(C#N)cc5F)n4)CC3)n(C)c2c1 ; COC(=O)c1cc(O[C@@H](C)F)c2nc(CN3CCC(c4cccc(OCc5ccc(C#N)cc5F)n4)CC3)n(C)c2c1]

2 was separated by SFC (column: Daicel ChiralPak IG (250*30 mm, 10 um); mobile phase: [CO₂-MeOH]; B %: 60%, isocratic elution mode). 3A (98 mg, 158.34 μmol, 30.89% yield, 93% purity) was obtained as a white solid. 3B (100 mg, 158.09 μmol, 30.85% yield, 91% purity) was obtained as a white solid.

LCMS: RT=0.462 min, MS cal.: 575.23, [M+H]⁺=576.2

¹H NMR (400 MHz, CHCl3-d) δ=7.90 (s, 1H), 7.69 (s, 1H), 7.67-7.58 (m, 1H), 7.58-7.50 (m, 1H), 7.45 (br d, J=8.4 Hz, 1H), 7.37 (br d, J=9.2 Hz, 1H), 6.76 (br d, J=7.2 Hz, 1H), 6.69-6.61 (m, 1H), 6.40-6.32 (m, 1H), 6.36 (br d, J=5.2 Hz, 1H), 5.51 (s, 2H), 3.99 (br s, 3H), 4.05-3.85 (m, 6H), 2.96 (br d, J=1.2 Hz, 2H), 2.60 (br d, J=2.8 Hz, 1H), 2.41-2.17 (m, 2H), 1.58 (br d, J=7.2 Hz, 2H), 1.92-1.45 (m, 9H)

### General Procedure for Preparation of Compound 312:

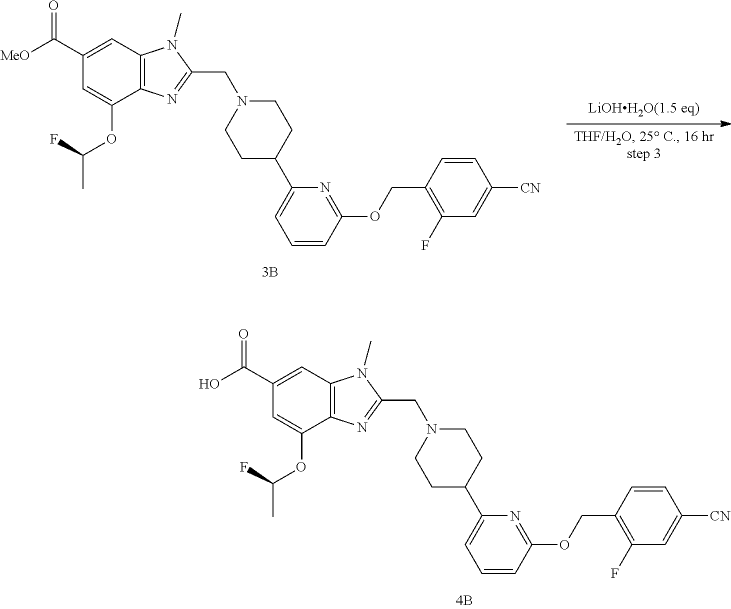 [SMILES: COC(=O)c1cc(O[C@@H](C)F)c2nc(CN3CCC(c4cccc(OCc5ccc(C#N)cc5F)n4)CC3)n(C)c2c1 ; "LiOH·H2O(1.5 eq)" ; "THF/H2O, 25° C., 16 hrstep 3" ; C[C@@H](F)Oc1cc(C(=O)O)cc2c1nc(CN1CCC(c3cccc(OCc4ccc(C#N)cc4F)n3)CC1)n2C]

To a solution of 3B (90 mg, 156.36 μmol, 1 eq) in THF (1.4 mL) was added LiOH·H₂O (9.84 mg, 234.54 μmol, 1.5 eq) in H2O (0.6 mL). The mixture was stirred at 25° C. for 16 hr. The mixture was monitored by LC-MS showed ˜25% of 3B remained. Several new peaks were shown on LC-MS and ˜64% of desired product was detected. The reaction mixture was used directly in the next step. The reaction mixture was purified by prep-HPLC (column: Waters Xbridge BEH C18 100*30 mm*10 um; mobile phase: [H₂O (10 mM NH₄HCO₃)-ACN]; gradient: 20%-70% B over 8.0 min). (R)-2-((4-(6-((4-Cyano-2-fluorobenzyl)oxy)pyridin-2-yl)piperidin-1-yl)methyl)-4-(1-fluoroethoxy)-1-methyl-1H-benzo[d]imidazole-6-carboxylic acid (28.98 mg, 48.37 μmol, 30.93% yield, 93.73% purity) was obtained as a white solid.

LCMS: RT=2.289 min, MS cal.: 561.22, [M+H]⁺=562.2

HPLC: RT=10.571 min, purity: 93.73%

¹H NMR (400 MHz, CHCl3-d) δ=7.89 (s, 1H), 7.75 (s, 1H), 7.65-7.58 (m, 1H), 7.53 (t, J=8.0 Hz, 1H), 7.43 (d, J=8.0 Hz, 1H), 7.38-7.38 (m, 1H), 7.37-7.33 (m, 1H), 6.76 (d, J=7.2 Hz, 1H), 6.64 (d, J=8.0 Hz, 1H), 6.54-6.48 (m, 1H), 6.39-6.33 (m, 1H), 5.49 (s, 2H), 3.98 (s, 3H), 3.97 (br s, 2H), 3.10-3.01 (m, 2H), 2.67-2.57 (m, 1H), 2.41-2.31 (m, 2H), 1.93-1.73 (m, 7H)

### General Procedure for Preparation of Compound 313:

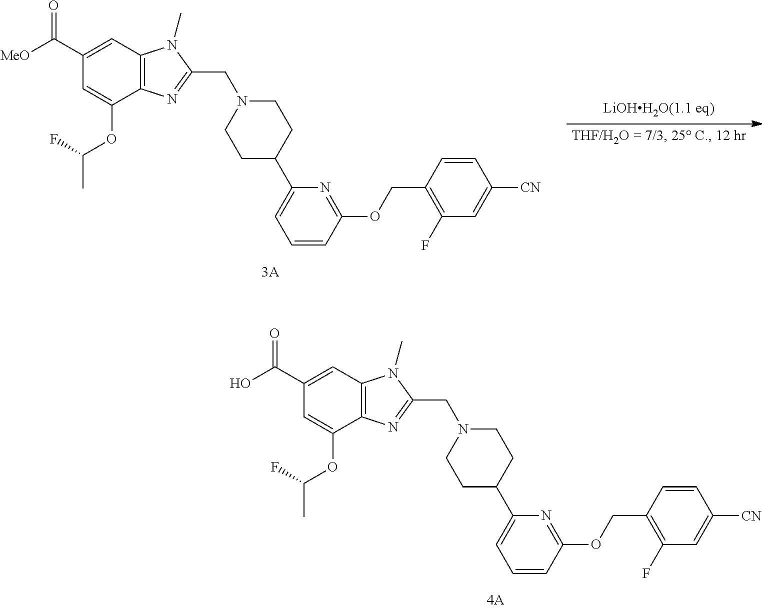

A mixture of 3A (85.00 mg, 147.67 μmol, 1 eq), LiOH·H₂O (6.82 mg, 162.44 μmol, 1.1 eq), in THF (0.7 mL) and H₂O (0.3 mL) was degassed and purged with N₂ for 3 times, and then the mixture was stirred at 25° C. for 12 hr under N₂ atmosphere. LC-MS (ET83888-183, product, RT=1.074 min) showed 3A as consumed completely and one main peak with desired mass was detected. The reaction was purified directly. The residue was purified by prep-HPLC (column: Waters Xbridge Prep OBD C18 150*40 mm*10 um; mobile phase: [H₂O (10 mM NH₄HCO₃)-ACN]; gradient: 20%-70% B over 8.0 min). (S)-2-((4-(6-((4-Cyano-2-fluorobenzyl)oxy)pyridin-2-yl)piperidin-1-yl)methyl)-4-(1-fluoroethoxy)-1-methyl-1H-benzo[d]imidazole-6-carboxylic acid (27.2 mg, 46.59 μmol, 31.55% yield, 96.20% purity) was obtained as a white solid.

LCMS: RT=1.074 min, MS cal.: 561.22, [M+H]⁺=562.1

HPLC: RT=11.54 min, purity: 96.20. %

SFC: ee %=100%

¹H NMR (400 MHz, CHCl3-d) δ=8.01-7.97 (m, 1H), 7.70-7.64 (m, 2H), 7.62-7.51 (m, 3H), 6.83 (d, J=7.2 Hz, 1H), 6.68 (d, J=8.4 Hz, 1H), 6.44 (dq, J=62.4, 4.8 Hz, 1H), 5.50 (s, 2H), 4.00 (s, 3H), 3.95 (s, 2H), 3.06 (br dd, J=3.2, 11.2 Hz, 2H), 2.64 (br t, J=7.2 Hz, 1H), 2.42-2.33 (m, 2H), 1.89-1.81 (m, 4H), 1.80-1.71 (m, 3H), 2.78 (m, 1H), 2.21-2.09 (m, 2H), 1.83-1.54 (m, 4H)

### Example A48—Preparation of Compound 314. (S)-2-((4-(3-(4-Cyano-2-fluorophenyl)-2,3-dihydrobenzo[b][1,4]dioxin-5-yl)piperidin-1-yl)methyl)-4-methoxy-1-methyl-1H-benzo[d]imidazole-6-carboxylic acid (Compound 314) (3)

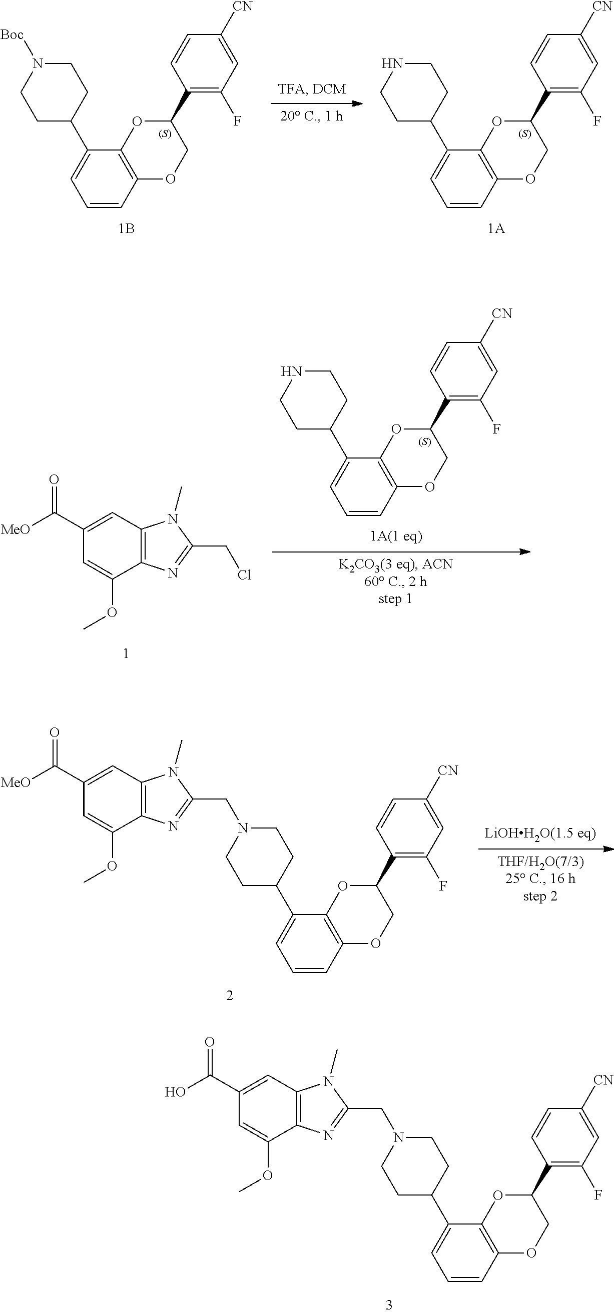

### General Procedure for Preparation of Intermediate 1A:

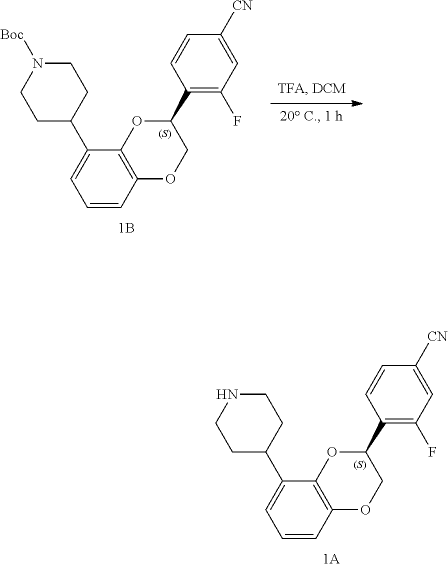

To a solution of 1B (90 mg, 205.25 μmol, 1 eq) in DCM (1.2 mL) was added TFA (0.3 mL). The mixture was stirred at 20° C. for 1 hr. LC-MS showed 1B was consumed completely and one main peak with desired mass was detected. The mixture was concentrated under reduced pressure. Without purification. 1A (90 mg, 198.94 μmol, 96.93% yield, TFA) was obtained as yellow oil.

LCMS: RT=0.470 min, MS cal.: 338.14, [M+H]⁺=339.3

### General Procedure for Preparation of Intermediate 2:

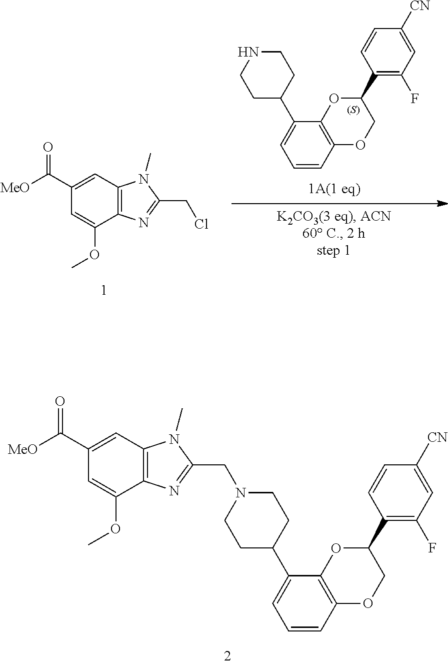

To a solution of 1 (80 mg, 236.42 μmol, 1 eq) in ACN (2 mL) was added K₂CO₃ (98.03 mg, 709.27 μmol, 3 eq) and 1A (63.53 mg, 236.42 μmol, 1 eq). The mixture was stirred at 60° C. for 2 hr. LCMS showed 1 was consumed completely and one main peak with desired mass was detected. The residue was poured into ice-water (5 mL) and stirred for 10 min. The aqueous phase was extracted with ethyl acetate (10 mL*3). The combined organic phase was washed with brine (10 mL), dried with anhydrous Na₂SO₄, filtered and concentrated in vacuum. The residue was purified by prep-TLC (Ethyl acetate, Rf=0.18). 2 (90 mg, 157.73 μmol, 66.71% yield) was obtained as a white solid.

LCMS: RT=0.654 min, MS cal.: 570.23, [M+H]⁺=571.4

SFC: RT=1.325 min, ee %=99.44

¹H NMR (400 MHz, CHCl3-d) δ=7.80 (d, J=1.2 Hz, 1H), 7.64-7.56 (m, 2H), 7.47-7.39 (m, 2H), 6.93-6.80 (m, 3H), 5.50 (dd, J=1.6, 7.2 Hz, 1H), 4.45 (dd, J=2.4, 11.0 Hz, 1H), 4.08 (s, 3H), 4.03-3.99 (m, 3H), 3.98 (s, 3H), 3.79-3.73 (m, 3H), 3.27-3.10 (m, 1H), 3.06-2.98 (m, 1H), 2.97-2.88 (m, 1H), 2.68-2.40 (m, 2H), 1.99-1.90 (m, 2H), 1.86 (td, J=3.2, 6.8 Hz, 4H)

### General Procedure for Preparation of Compound 314:

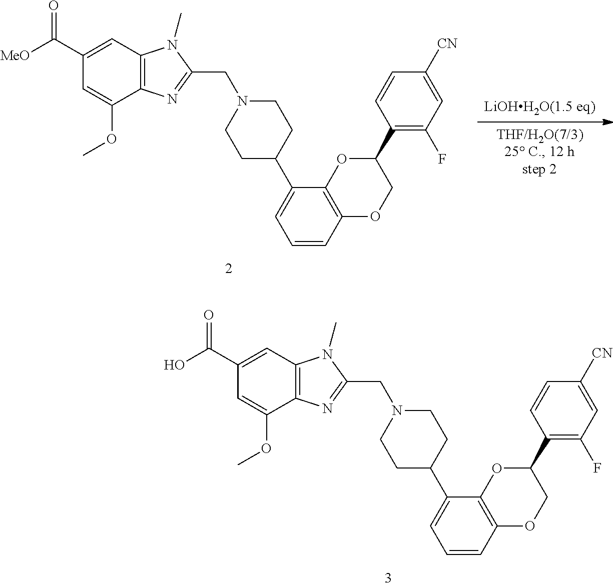 [SMILES: COC(=O)c1cc(OC)c2nc(CN3CCC(c4cccc5c4O[C@@H](c4ccc(C#N)cc4F)CO5)CC3)n(C)c2c1 ; "LiOH·H2O(1.5 eq)" ; "THF/H2O(7/3)25° C., 12 hstep 2" ; COc1cc(C(=O)O)cc2c1nc(CN1CCC(c3cccc4c3O[C@@H](c3ccc(C#N)cc3F)CO4)CC1)n2C]

To a solution of 2 (80 mg, 140.20 μmol, 1 eq) in THF (1.4 mL) and H₂O (0.6 mL) was added LiOH·H₂O (8.82 mg, 210.30 μmol, 1.5 eq). The mixture was stirred at 25° C. for 12 hr. HPLC showed 2 was consumed completely and one main peak with desired mass was detected. The mixture was purified directly. The crude was purified by prep-HPLC (column: Waters Xbridge Prep OBD C18 150*40 mm*10 um; mobile phase: [H₂O (10 mM NH₄HCO₃)-ACN]; gradient: 25%-55% B over 8.0 min). (S)-2-((4-(3-(4-Cyano-2-fluorophenyl)-2,3-dihydrobenzo[b][1,4]dioxin-5-yl)piperidin-1-yl)methyl)-4-methoxy-1-methyl-1H-benzo[d]imidazole-6-carboxylic acid (30.29 mg, 54.36 μmol, 38.77% yield, 99.89% purity) was obtained as a white solid.

HPLC: RT=1.879 min, purity: 70.53%

LCMS: RT=1.888 min, MS cal.: 556.2, 559.2, [M+H]⁺=557.3

HPLC: RT=9.333 min, purity: 99.86%

SFC: RT=3.526 min, ee %=98.76

¹H NMR (400 MHz, DMSO-d6) δ=7.99 (dd, J=1.2, 10.4 Hz, 1H), 7.84 (dd, J=1.2, 8.0 Hz, 1H), 7.73-7.64 (m, 2H), 7.27 (s, 1H), 6.89-6.75 (m, 3H), 5.59 (dd, J=2.0, 7.2 Hz, 1H), 4.47 (dd, J=2.0, 11.2 Hz, 1H), 4.11 (dd, J=7.6, 11.6 Hz, 1H), 3.92 (s, 3H), 3.85 (s, 3H), 3.82-3.71 (m, 2H), 2.97-2.82 (m, 3H), 2.22-2.09 (m, 2H), 1.79 (br d, J=11.6 Hz, 1H), 1.74-1.57 (m, 3H)

### Example A49—Preparation of Compound 294 and Compound 315. (S)-2-((4-(3-(4-Cyano-2-fluorophenyl)-2,3-dihydrobenzo[b][1,4]dioxin-5-yl)piperidin-1-yl)methyl)-4-(2-fluoroethoxy)-1-methyl-1H-benzo[d]imidazole-6-carboxylic acid (Compound 294) (11A) and (R)-2-((4-(3-(4-Cyano-2-fluorophenyl)-2,3-dihydrobenzo[b][1,4]dioxin-5-yl)piperidin-1-yl)methyl)-4-(2-fluoroethoxy)-1-methyl-1H-benzo[d]imidazole-6-carboxylic acid (Compound 315) (11B)

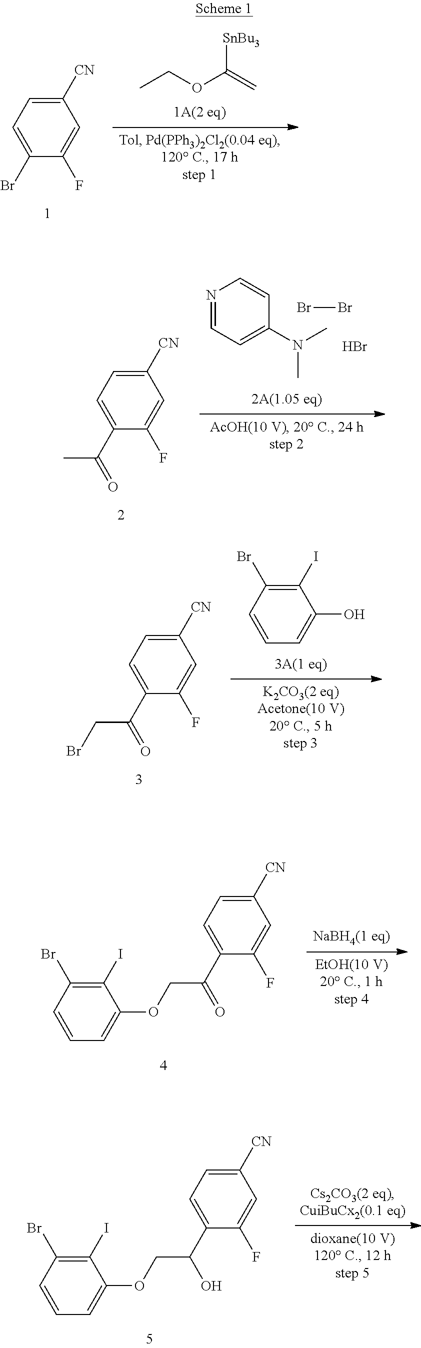

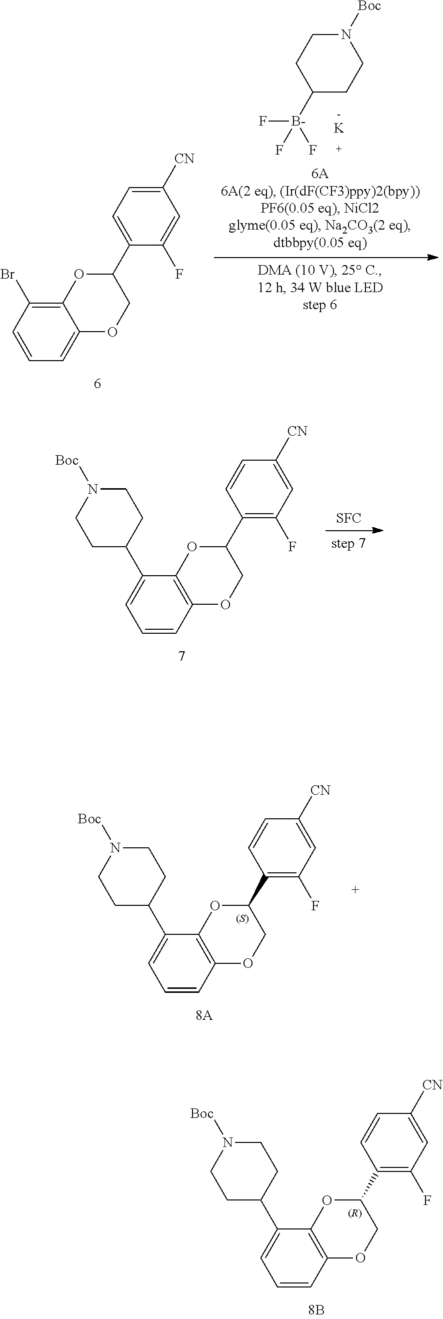

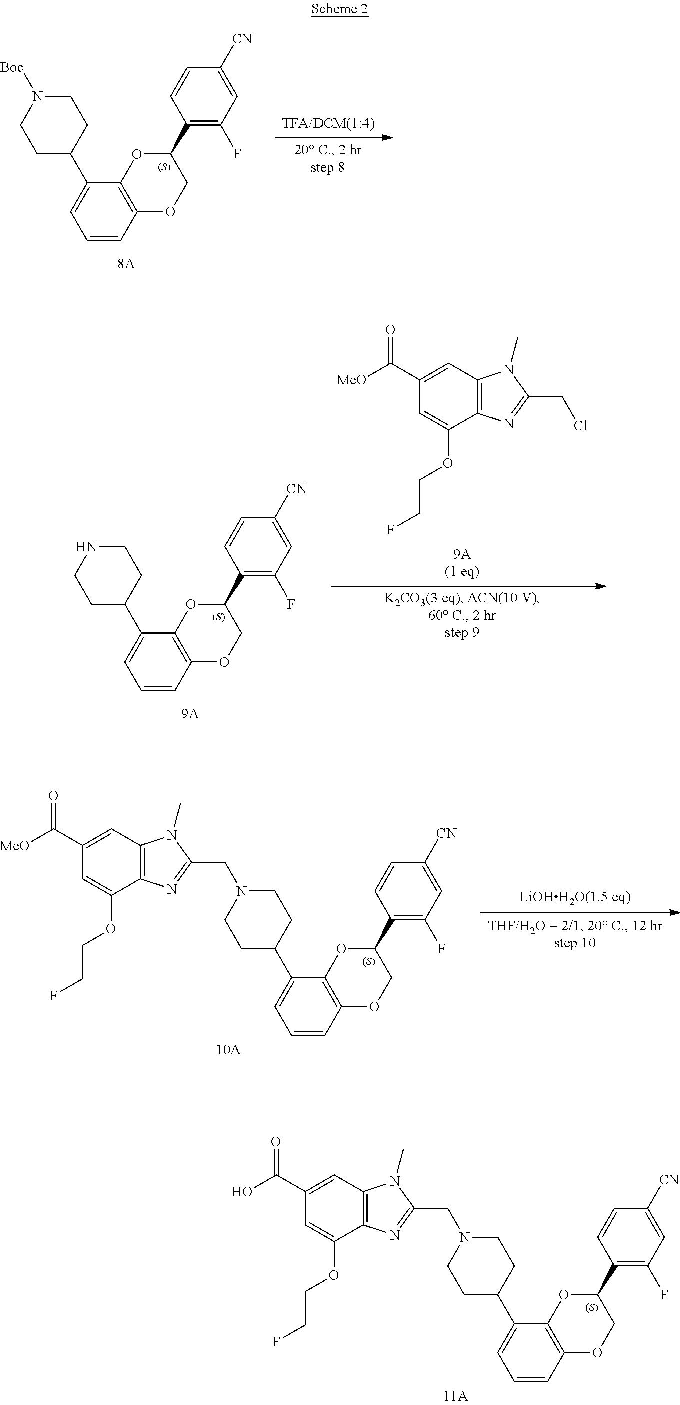

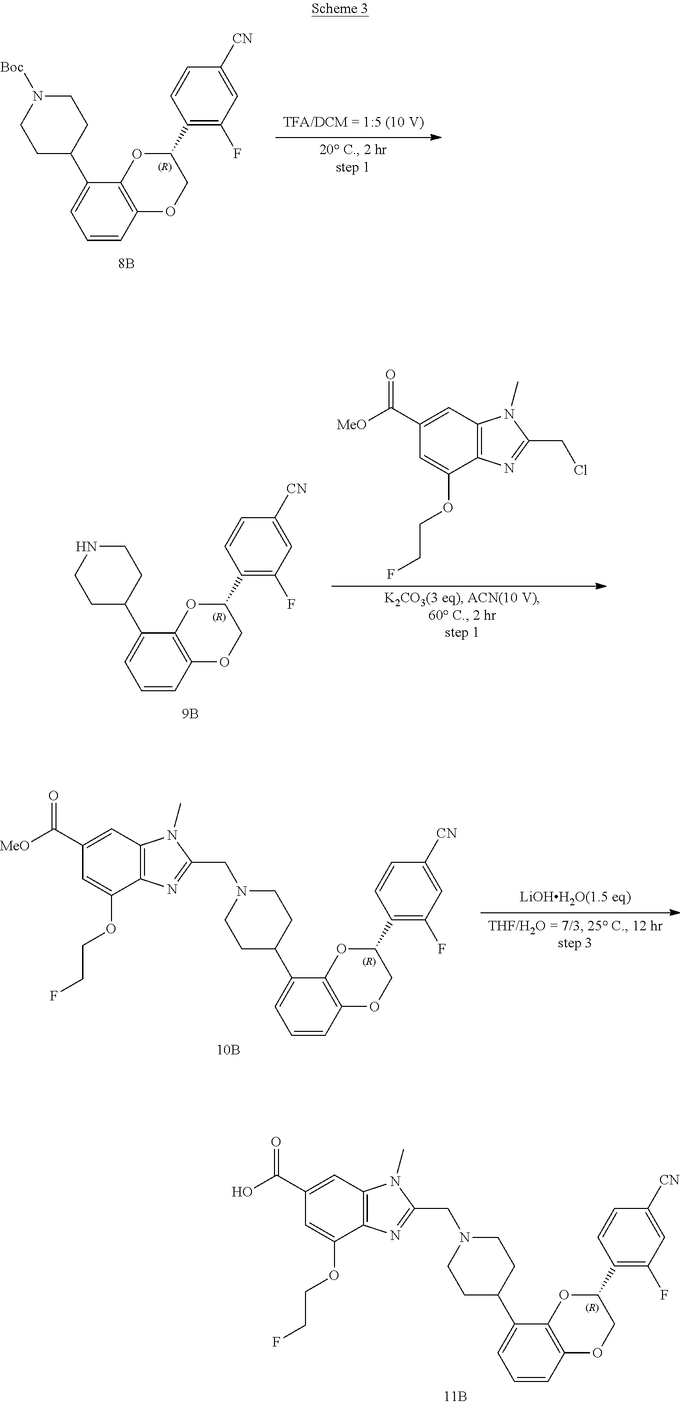

### General Procedure for Preparation of Intermediate 2:

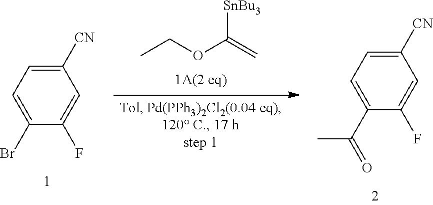

To a solution of 1 (25 g, 125.00 mmol, 1 eq) in Toluene (375 mL) was added 1A (49.66 g, 137.49 mmol, 46.45 mL, 1.1 eq) and Pd(PPh₃)₂Cl₂ (3.51 g, 5.00 mmol, 0.04 eq) at 20° C. The mixture was stirred for 5 hr at 120° C. under N₂ and then added 1A (40.63 g, 112.50 mmol, 38.01 mL, 0.9 eq) at 20° C. and stirred for 12 hr at 120° C. under N₂. HPLC showed that the starting material was consumed. The mixture was cooled down to 20° C. and added KF (250 ml, 2M) at 20° C. The mixture was stirred for 30 min and then added HCl (500 ml, 6M)) and stirred for 2 hr at 70° C. under N₂. The mixture was extracted with EA (800 ml, 500 ml) and washed with brine (300 ml) and concentrated to give the residue. The crude product was purified by column chromatography (SiO₂, PE/EA=100:1˜3:1). 2 (18 g, 110.33 mmol, 88.27% yield) was obtain as a white solid.

¹H NMR (400 MHz, DMSO-d6) δ=8.09 (dd, J=1.2, 10.2 Hz, 1H), 8.01 (t, J=8.0 Hz, 1H), 7.87 (dd, J=1.2, 8.0 Hz, 1H), 2.67 (d, J=4.0 Hz, 3H)

### General Procedure for Preparation of Intermediate 3:

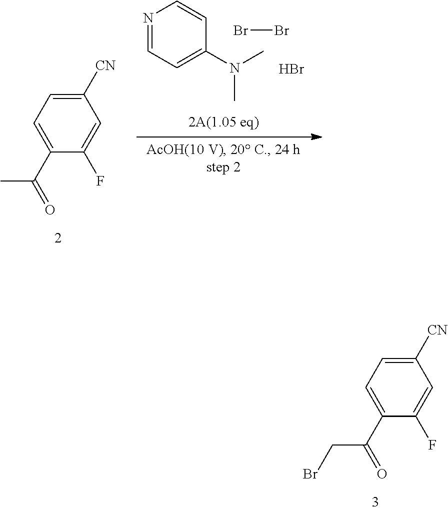

To a solution of 2 (10 g, 61.29 mmol, 1 eq) in AcOH (100 mL) was added 2A (23.35 g, 64.36 mmol, 1.05 eq) at 20° C. The mixture was stirred for 24 hr at 20° C. under N₂. HPLC (ET87376-30-P1A1) showed that the starting material was consumed. The mixture was filtered to give the residue. Page on ET87376-24 was combined to page on ET87376-30 for purification. The crude product was triturated with water (50 ml). 3 (14.2 g, 58.67 mmol, 53.17% yield) was obtain as a white solid.

¹H NMR (400 MHz, DMSO-d6) δ=8.14-8.02 (m, 2H), 7.88 (dd, J=1.2, 8.0 Hz, 1H), 4.90 (d, J=2.0 Hz, 2H)

### General Procedure for Preparation of Intermediate 4:

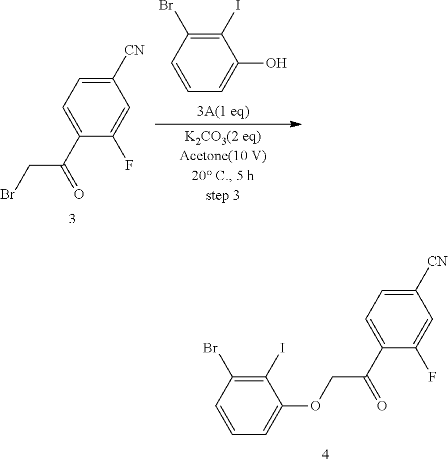

To a solution of 3 (13 g, 53.71 mmol, 1 eq) in acetone (130 mL) was added K₂CO₃ (14.85 g, 107.42 mmol, 2 eq) and 3A (16.05 g, 53.71 mmol, 1 eq) at 20° C. The mixture was stirred for 5 hr at 20° C. under N₂. HPLC (ET87376-34-p1a1) showed that the starting material was consumed. The mixture was poured into water (1 L) and extracted with EA (IL, 500 ml) and washed with brine (500 ml) and dried over Na₂SO₄ and concentrated to give the residue. The crude product was triturated with MTBE (30 ml) and filtered to give the filter cake. 4 (16 g, 34.78 mmol, 64.76% yield) was obtain as a gray solid.

¹H NMR (400 MHz, CHCl3-d) δ=8.04 (dd, J=6.8, 8.0 Hz, 1H), 7.61 (dd, J=1.2, 8.0 Hz, 1H), 7.54 (dd, J=1.2, 10.0 Hz, 1H), 7.33 (dd, J=1.2, 8.0 Hz, 1H), 7.17 (t, J=8.0 Hz, 1H), 6.62 (dd, J=1.0, 8.4 Hz, 1H), 5.22 (d, J=2.8 Hz, 2H)

### General Procedure for Preparation of Intermediate 5:

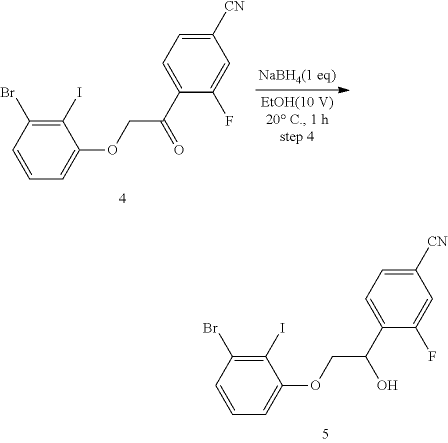 [SMILES: N#Cc1ccc(C(=O)COc2cccc(Br)c2I)c(F)c1 ; N#Cc1ccc(C(O)COc2cccc(Br)c2I)c(F)c1]

To a solution of 4 (14 g, 30.43 mmol, 1 eq) in EtOH (140 mL) was added NaBH₄ (1.15 g, 30.43 mmol, 1 eq) at 20° C. The mixture was stirred for 1 hr at 20° C. under N₂. TLC (PE/EA=3:1, Rf=0.5) showed that the starting material was consumed. The mixture was poured into sat NH₄Cl aqueous (500 ml) and extracted with EA (500 ml, 300 ml). The organic phase was washed with brine (200 ml) and concentrated to give the residue. The crude product was purified by column chromatography (SiO₂, PE/EA=100:1˜1:1). 5 (11 g, 23.81 mmol, 78.23% yield) was obtained as a white solid.

¹H NMR (400 MHz, DMSO-d6) δ=7.87 (t, J=7.2 Hz, 1H), 7.83 (dd, J=1.2, 10.0 Hz, 1H), 7.74 (d, J=8.0 Hz, 1H), 7.36-7.23 (m, 2H), 6.98 (dd, J=1.6, 7.8 Hz, 1H), 6.06 (d, J=4.8 Hz, 1H), 5.29 (q, J=5.2 Hz, 1H), 4.28-4.16 (m, 2H).

### General Procedure for Preparation of Intermediate 6:

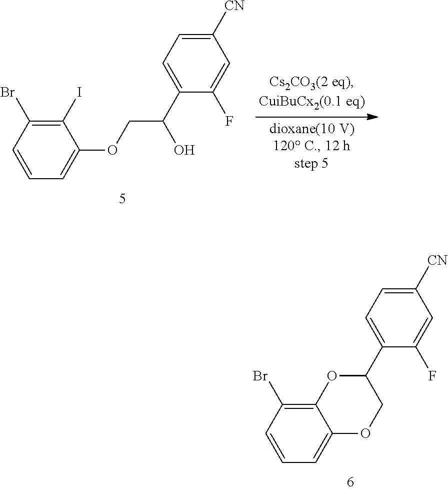 [SMILES: N#Cc1ccc(C(O)COc2cccc(Br)c2I)c(F)c1 ; N#Cc1ccc(C2COc3cccc(Br)c3O2)c(F)c1]

To a solution of 5 in dioxane (50 mL) was added dicesium; carbonate (7.05 g, 21.64 mmol, 2 eq) and bis[(1Z)-2-methyl-1-(2-oxocyclohexylidene) propoxy] copper (430.68 mg, 1.08 mmol, 0.1 eq) at 20° C. The mixture was stirred for 12 hr at 120° C. under N₂. TLC (PE/EA=5:1, Rf-0.5) showed that the starting material was consumed. The mixture was filtered to give the filtrate which was concentrated to give the residue. The crude product was purified by column chromatography (SiO₂, PE/EA=100:1, 1:1). 6 (1.6 g, 4.79 mmol, 44.25% yield) was obtained as a white solid.

¹H NMR (400 MHz, DMSO-d₆) δ=8.01 (d, J=10.4 Hz, 1H), 7.84 (d, J=7.8 Hz, 1H), 7.70 (t, J=7.6 Hz, 1H), 7.22 (dd, J=1.0, 7.8 Hz, 1H), 6.99 (dd, J=1.0, 8.0 Hz, 1H), 6.87 (t, J=8.0 Hz, 1H), 5.72 (dd, J=2.0, 7.6 Hz, 1H), 4.56 (dd, J=2.4, 11.8 Hz, 1H), 4.22 (dd, J=7.8, 11.8 Hz, 1H)

### General Procedure for Preparation of Intermediate 7:

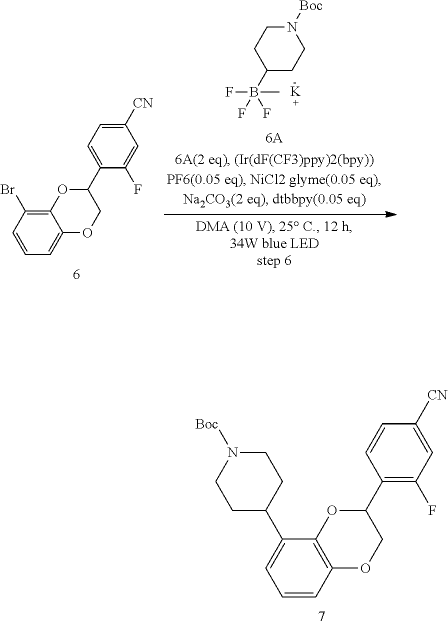

To a solution of 6 in DMA (8 mL) was added 6A (697.10 mg, 2.39 mmol, 2 eq), dichloronickel; 1,2-dimethoxyethane (13.15 mg, 59.86 μmol, 0.05 eq), Na₂CO₃ (253.76 mg, 2.39 mmol, 2 eq), dtbbpy (16.06 mg, 59.86 μmol, 0.05 eq),bis[3,5-difluoro-2-[5-(trifluoromethyl)-2-pyridyl]phenyl]iridium(1+); 2-(2-pyridyl)pyridine; hexafluorophosphate (60.44 mg, 59.86 μmol, 0.05 eq) at 25° C. under Ar and stirred for 12 hr under Ar 34W blue LED. TLC (PE/EA=5:1, Rf-0.6) showed that the starting material was consumed. The mixture was poured into water (10 ml) and extracted with EA (10 ml, 5 ml) and washed with brine (20 ml) and concentrated to give the residue. The crude product was purified by column chromatography (SiO₂, PE/EA=100:1˜3:1). 7 (0.35 g, 798.19 μmol, 66.68% yield) was obtained as a white solid.

¹H NMR (400 MHz, DMSO-d₆) δ=8.02-7.95 (m, 1H), 7.82 (d, J=8.0 Hz, 1H), 7.68 (t, J=7.6 Hz, 1H), 6.88-6.78 (m, 3H), 5.60 (dd, J=2.0, 7.2 Hz, 1H), 4.48 (dd, J=2.2, 11.6 Hz, 1H), 4.16-4.02 (m, 3H), 3.01 (br t, J=12.0 Hz, 1H), 2.77 (br s, 2H), 1.78 (br d, J=12.4 Hz, 1H), 1.74-1.66 (m, 1H), 1.60-1.42 (m, 2H), 1.38 (s, 9H)

### General Procedure for Preparation of Intermediate 8A & Intermediate 8B:

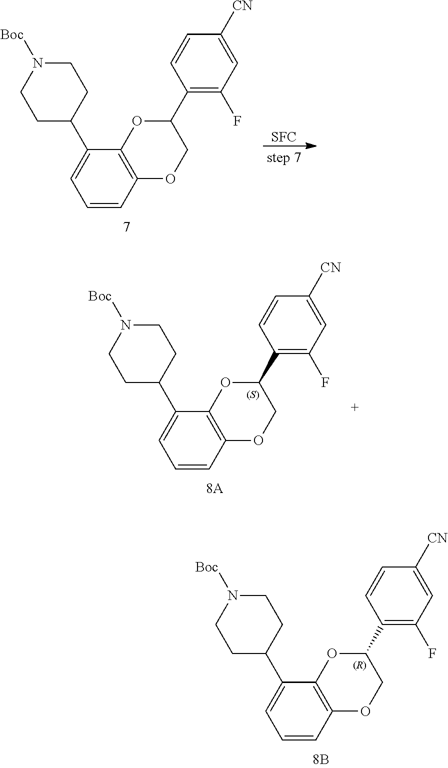

7 (0.35 g, 798.19 μmol, 1 eq) was separated by SFC (column: ChiralPak IH, 250*50 mm, 10 um; mobile phase: [CO₂-IPA (0.1% NH₃H₂O)]; B %: 20%, isocratic elution mode). 8A (0.19 g, 433.31 μmol, 54.29% yield) was obtain as a white solid.

SFC: RT=1.186 min, ee %=99.66%

¹H NMR (400 MHz, DMSO-d₆) δ=8.06 (dd, J=1.2, 10.4 Hz, 1H), 7.88 (d, J=7.6 Hz, 1H), 7.74 (t, J=7.6 Hz, 1H), 6.94-6.83 (m, 3H), 5.66 (dd, J=2.0, 7.2 Hz, 1H), 4.54 (dd, J=2.4, 11.6 Hz, 1H), 4.23-4.05 (m, 3H), 3.12-3.03 (m, 1H), 2.95-2.72 (m, 2H), 1.89-1.72 (m, 2H), 1.66-1.49 (m, 2H), 1.45 (s, 9H)

8B (0.17 g, 387.69 μmol, 48.57% yield) was obtain as a white solid.

SFC: RT=1.433 min, ee %=99.52%

¹H NMR (400 MHz, DMSO-d₆) δ=8.06 (dd, J=1.2, 10.4 Hz, 1H), 7.88 (d, J=7.6 Hz, 1H), 7.74 (t, J=7.6 Hz, 1H), 6.94-6.83 (m, 3H), 5.66 (dd, J=2.0, 7.2 Hz, 1H), 4.54 (dd, J=2.4, 11.6 Hz, 1H), 4.23-4.05 (m, 3H), 3.12-3.03 (m, 1H), 2.95-2.72 (m, 2H), 1.89-1.72 (m, 2H), 1.66-1.49 (m, 2H), 1.45 (s, 9H)

### General Procedure for Preparation of Intermediate 9:

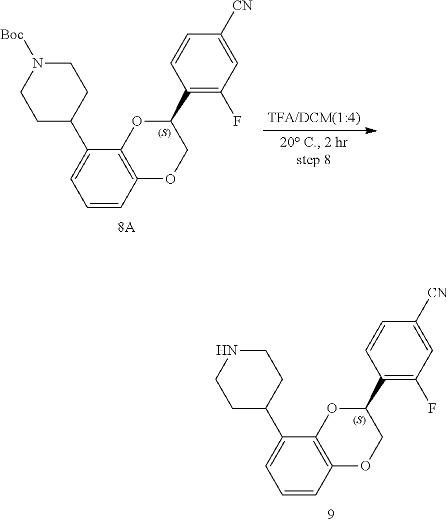

To a solution of 8A (60.00 mg, 136.83 μmol, 1 eq) in DCM (2.4 mL) was added TFA (0.6 mL) at 20° C. The mixture was stirred for 2 hr at 20° C. under N₂. LCMS (ET87376-53-p1a1) showed that the starting material was consumed. The mixture was concentrated to give the residue. The crude product was used to the next step without purification. 9 (0.062 g, crude, TFA) was obtain as a light yellow oil LCMS: RT=0.403 min, MS cal.: 338.38, [M+H]⁺=339.2

### General Procedure for Preparation of Intermediate 10:

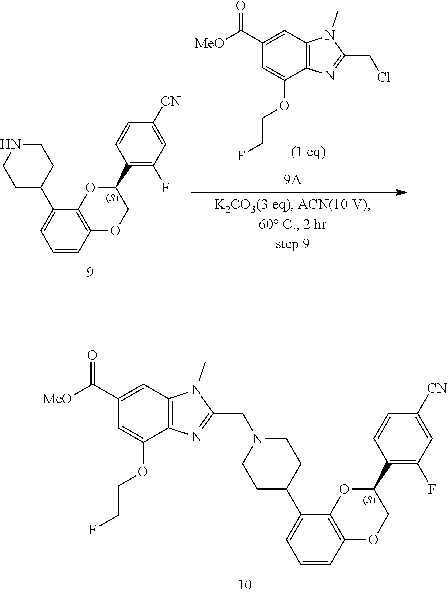

To a solution of 9 (61.90 mg, 136.83 μmol, 1 eq, TFA) in ACN (2 mL) was added K₂CO₃ (56.73 mg, 410.48 μmol, 3 eq) and 9A (41.15 mg, 136.83 μmol, 1 eq) at 20° C. The mixture was stirred for 2 hr at 60° C. under N₂. LCMS (Rt=0.468, MS+1=603.4) showed that the starting material was consumed. The mixture was poured into water (5 ml) and extracted with EA (10 ml, 5 ml) and washed with brine (3 ml), dried over Na₂SO₄, filtered, and concentrated to give the residue. The crude product was purified by prep-TLC (PE/EA=0:1). 10 (0.07 g, 116.16 μmol, 84.89% yield) was obtained as a white solid.

LCMS: RT=0.468 min, MS cal.: 602.64, [M+H]⁺=603.4

SFC: RT=2.315 min, ee %=99.49%

¹H NMR (400 MHz, DMSO-d₆) δ=8.06 (br d, J=10.0 Hz, 1H), 7.96-7.85 (m, 2H), 7.74 (t, J=7.6 Hz, 1H), 7.35 (s, 1H), 6.95-6.80 (m, 3H), 5.65 (br d, J=6.0 Hz, 1H), 4.94 (dt, J=48, 3.6 Hz, 2H), 4.66-4.47 (m, 3H), 4.18 (br dd, J=7.6, 11.6 Hz, 1H), 3.96 (d, J=9.2 Hz, 6H), 3.87 (br s, 2H), 3.06-2.87 (m, 3H), 2.32-2.17 (m, 2H), 1.86 (br d, J=11.2 Hz, 1H), 1.81-1.64 (m, 3H)

### General Procedure for Preparation of Compound 294:

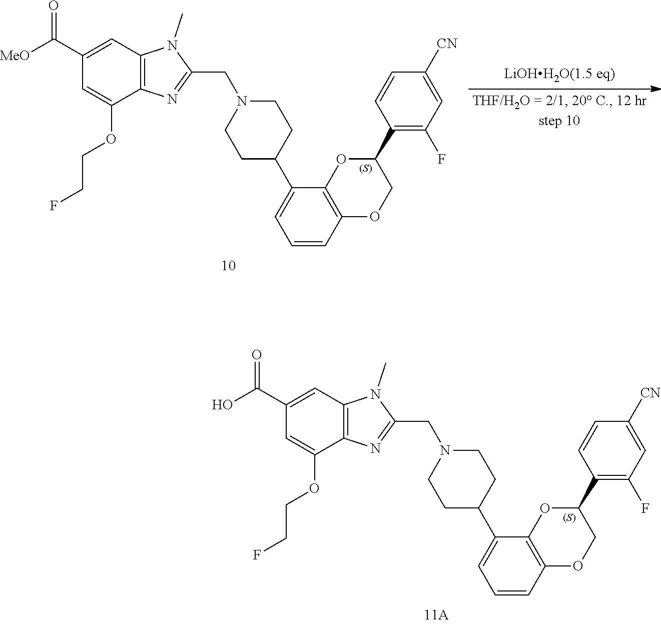

To a solution of 10 (0.05 g, 82.97 μmol, 1 eq) in THF (1 mL) was added LiOH·H₂O (5.22 mg, 124.46 μmol, 1.5 eq) in H₂O (0.5 mL) at 20° C. The mixture was stirred for 12 hr at 20° C. under N₂. LCMS showed that the starting material was consumed. The mixture was diluent with THF (1 ml) and purified by prep-HPLC (column: Waters Xbridge Prep OBD C18 150*40 mm*10 um; mobile phase: [H2O (10 mM NH4HCO3)-ACN]; gradient: 20%-65% B over 8.0 min). (S)-2-((4-(3-(4-Cyano-2-fluorophenyl)-2,3-dihydrobenzo[b][1,4]dioxin-5-yl)piperidin-1-yl)methyl)-4-(2-fluoroethoxy)-1-methyl-1H-benzo[d]imidazole-6-carboxylic acid (29.96 mg, 50.90 μmol, 61.35% yield) was obtain as a white solid.

LCMS: RT=0.462 min, MS cal.: 588.61, [M+H]⁺=589.2

SFC: RT=4.433 min, ee %=99.52%

¹H NMR (400 MHz, DMSO-d6) δ=8.03-7.98 (m, 1H), 7.87-7.83 (m, 1H), 7.83-7.80 (m, 1H), 7.68 (t, J=7.8 Hz, 1H), 7.30-7.27 (m, 1H), 6.86-6.77 (m, 3H), 5.62-5.57 (m, 1H), 4.88 (dt, J=48, 3.6 Hz, 2H), 4.56-4.43 (m, 3H), 4.12 (dd, J=7.6, 11.2 Hz, 1H), 3.89 (s, 3H), 3.85-3.75 (m, 2H), 3.00-2.81 (m, 3H), 2.24-2.11 (m, 2H), 1.87-1.54 (m, 4H)

### General Procedure for Preparation of Intermediate 9B:

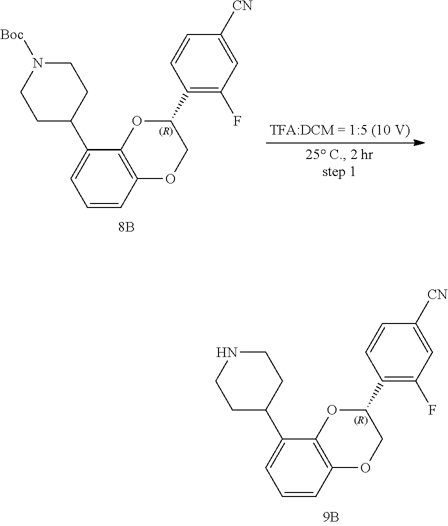

To a solution of 8B (75 mg, 171.04 μmol, 1 eq) in DCM (1 mL) was added TFA (0.2 ml). The mixture was stirred at 25° C. for 2 hr. LCMS showed 8B was consumed completely and one main peak with desired mass was detected. The mixture was concentrated under reduced pressure. Without purification. 9B (77 mg, crude, TFA) was obtained as a colorless oil.

LCMS: RT=0.395 min, MS cal.: 338.1/339.1, [M+1]⁺=339.1/340.1

### General Procedure for Preparation of Intermediate 10B:

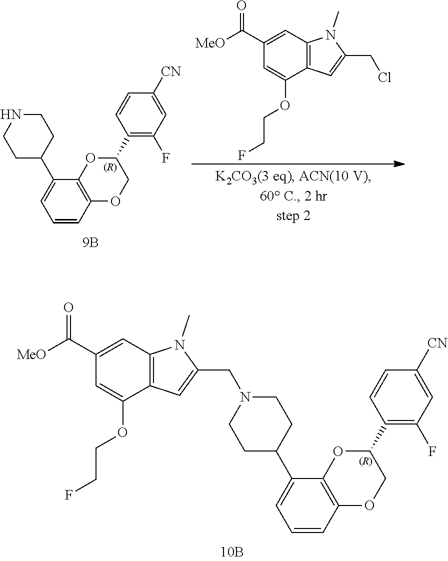

To a solution of 9B (77 mg, 170.20 μmol, 1 eq, TFA) in ACN (1 mL) was added K₂CO₃ (70.57 mg, 510.61 μmol, 3 eq) and 9A (51.18 mg, 170.20 μmol, 1 eq). The mixture was stirred at 60° C. for 2 hr. LCMS showed 9B was consumed completely and one main peak with desired mass was detected. The reaction mixture was diluted with H₂O 10 mL and extracted with EA 30 mL (10 mL*3). The combined organic layers were washed with brine 20 mL, dried over Na₂SO₄, filtered, and concentrated under reduced pressure to give a residue. The residue was purified by column chromatography (SiO₂, PE/EtOAc=1/0 to 1/1). 10B (60 mg, 93.59 μmol, 54.99% yield) was obtained as a white solid.

LCMS: RT=1.515 min, MS cal.: 602.2/603.2, [M+H]⁺=603.1/604.1

¹HNMR (400 MHz, DMSO-d₆) δ=8.01-7.97 (m, 1H), 7.85-7.81 (m, 2H), 7.67 (t, J=7.6 Hz, 1H), 7.29-7.27 (m, 1H), 6.85-6.76 (m, 3H), 5.61-5.55 (m, 1H), 4.82 (dt, J=48, 4.0 Hz, 2H), 4.55-4.44 (m, 3H), 4.10 (dd, J=7.6, 11.6 Hz, 1H), 3.89 (d, J=9.2 Hz, 6H), 3.80 (d, J=2.1 Hz, 2H), 2.96-2.80 (m, 3H), 2.22-2.12 (m, 2H), 1.79 (br d, J=11.9 Hz, 1H), 1.73-1.65 (m, 2H), 1.60 (br dd, J=3.2, 12.2 Hz, 1H)

### General Procedure for Preparation of Compound 315:

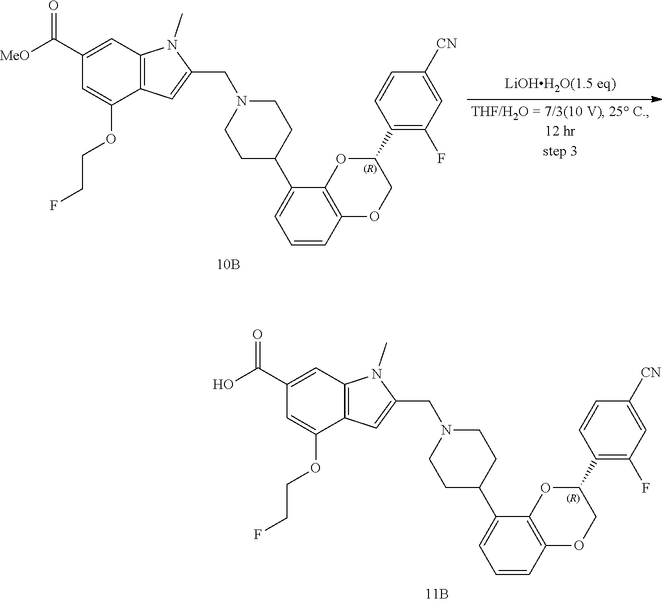

To a solution of 10B (50 mg, 82.97 μmol, 1 eq) in THF (0.7 mL) and H₂O (0.3 mL) was added LiOH·H₂O (5.22 mg, 124.46 μmol, 1.5 eq). The mixture was stirred at 25° C. for 12 hr. LCMS showed 16% of 10B remained. Several new peaks were shown on LCMS and 71% of desired Compound was detected. The mixture was added THF (0.1 mL). The residue was purified by prep-HPLC (neutral condition: column: Waters Xbridge Prep OBD C18 150*40 mm*10 um; mobile phase: [H₂O (10 mM NH₄HCO₃)-ACN]; gradient: 20%-50% B over 8.0 min). (R)-2-((4-(3-(4-Cyano-2-fluorophenyl)-2,3-dihydrobenzo[b][1,4]dioxin-5-yl)piperidin-1-yl)methyl)-4-(2-fluoroethoxy)-1-methyl-1H-benzo[d]imidazole-6-carboxylic acid (21.72 mg, 36.53 μmol, 44.03% yield) was obtained as a white solid.

LCMS: RT=2.626 min, MS cal.: 588.2/589.2, [M+H]⁺=589.3/590.3

HPLC: RT=10.624 min

¹HNMR (400 MHz, DMSO-d₆) δ=7.99 (dd, J=1.3, 10.2 Hz, 1H), 7.84 (dd, J=1.3, 8.0 Hz, 1H), 7.80 (d, J=0.8 Hz, 1H), 7.68 (t, J=7.6 Hz, 1H), 7.28 (d, J=1.0 Hz, 1H), 6.87-6.76 (m, 3H), 5.61-5.56 (m, 1H), 4.87 (dt, J=48, 4.0 Hz, 2H), 4.54-4.43 (m, 3H), 4.11 (dd, J=7.5, 11.6 Hz, 1H), 3.89 (s, 3H), 3.85-3.73 (m, 1H), 2.97-2.78 (m, 3H), 2.24-2.08 (m, 2H), 1.84-1.55 (m, 4H)

### Example A50—Preparation of Compound 295. 2-((4-(6-((4-Cyano-2-fluorobenzyl)oxy)pyridin-2-yl)piperidin-1-yl)methyl)-4-(ethoxy-1,1-d2)-1-methyl-1H-benzo[d]imidazole-6-carboxylic acid (Compound 295) (3)

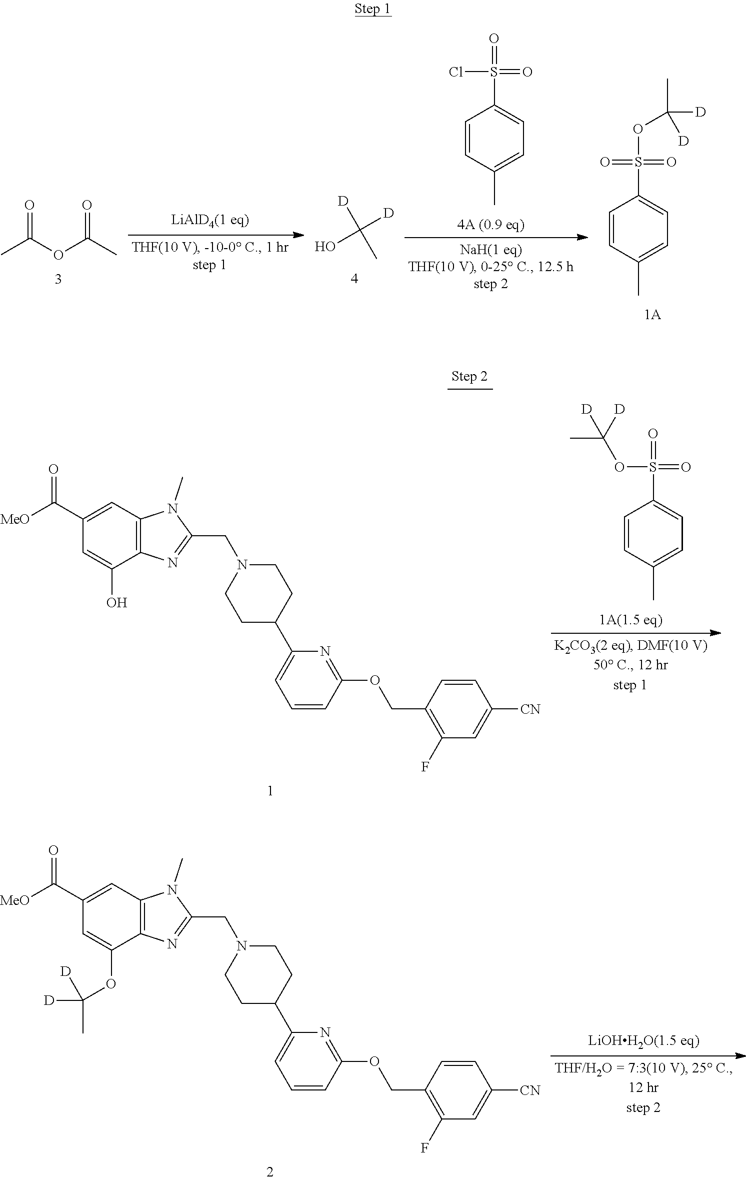

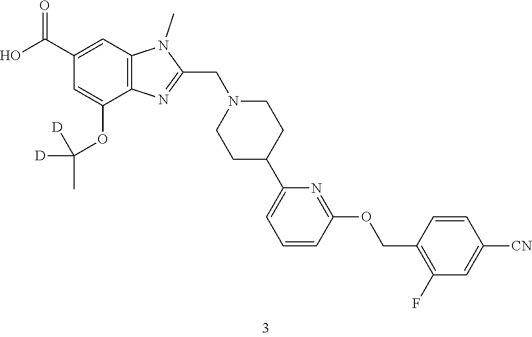 [SMILES: [2H]C([2H])(C)Oc1cc(C(=O)O)cc2c1nc(CN1CCC(c3cccc(OCc4ccc(C#N)cc4F)n3)CC1)n2C]

### General Procedure for Preparation of Intermediate 4:

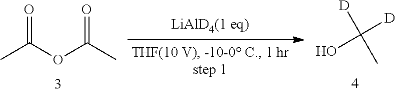

THF (25 mL) was charged to the three-necked round bottom flask, then LiAlD₄ (743.56 mg, 19.59 mmol, 1.01 mL, 1 eq) was added to the mixture at 0° C. At −10° C. (inner temperature), 3 (2 g, 19.59 mmol, 1.84 mL, 1 eq) in THF (3 mL) was added dropwise to the reaction mixture at −10° C. After the addition, the mixture was stirred at −10° C. for 1 hr. HNMR (ET62365-786-p1a) showed 3 was consumed completely. The reaction mixture was quenched by addition D₂O (2 mL) at 0° C., and then added NaOD (0.8 mL, 15% w/w). The reaction mixture was filtered, and the filter cake was washed by THF (20 mL). The filtrate was combined, dried over Na₂SO₄. 4 (42 g, ˜1.7% w/w) was obtained as a THF solution. It was used into the next step without further purification.

¹H NMR (400 MHz, CHCl3-d) δ=1.05 (s, 3H).

### General Procedure for Preparation of Intermediate 1A:

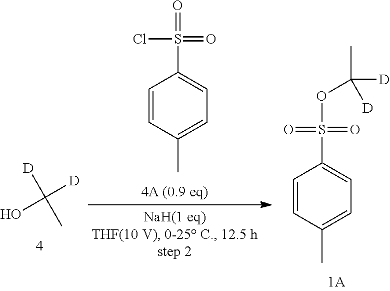

4 (16.8 g, 5.94 mmol, 1 eq) (in THF, 1.7% w/w) was charged to the three-necked round bottom flask at 25° C. At 0° C. (inner temperature), NaH (237.60 mg, 5.94 mmol, 60% purity, 1 eq) was added in portions to the reaction mixture at 0° C. After the addition, the mixture was stirred at 25° C. for 0.5 hr. Then the reaction mixture was added TosCl (1.02 g, 5.35 mmol, 0.9 eq), the mixture was stirred at 25° C. for 12 hr. TLC (Petroleum ether/Ethyl acetate=5/1, R_{f}=0.3) indicated 4 was consumed completely and one new spot formed. The reaction mixture was quenched by addition HCl (10 mL, 1M) at 0° C., and then extracted with DCM (10 mL*4). The combined organic layers were dried over Na₂SO₄, filtered and concentrated under reduced pressure to give a residue. The residue was purified by column chromatography (SiO₂, Petroleum ether/Ethyl acetate=100/1 to 0/1). 1A (0.75 g, 3.71 mmol, 62% yield) was obtained as a white oil.

¹H NMR (400 MHz, CHCl3-d) δ=7.80 (d, J=8.4 Hz, 2H), 7.35 (d, J=8.4 Hz, 2H), 4.11 (q, J=7.2 Hz, 1H), 2.46 (s, 3H), 1.29 (s, 3H)

### General Procedure for Preparation of Intermediate 2:

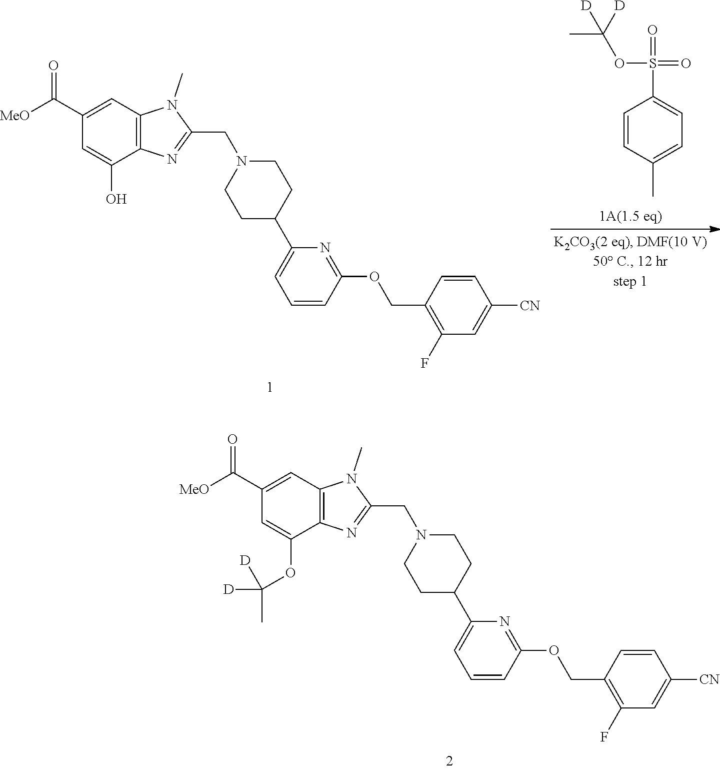 [SMILES: COC(=O)c1cc(O)c2nc(CN3CCC(c4cccc(OCc5ccc(C#N)cc5F)n4)CC3)n(C)c2c1 ; [2H]C([2H])(C)OS(=O)(=O)c1ccc(C)cc1 ; [2H]C([2H])(C)Oc1cc(C(=O)OC)cc2c1nc(CN1CCC(c3cccc(OCc4ccc(C#N)cc4F)n3)CC1)n2C]

To a solution of 1 (100 mg, 188.84 μmol, 1 eq) (Synthesized from Int 7) in DMF (1 mL) was added 1, 1-dideuterioethyl 4-methylbenzenesulfonate (57.29 mg, 283.25 μmol, 1.5 eq) and K₂CO₃ (52.20 mg, 377.67 μmol, 2 eq). The mixture was stirred at 50° C. for 12 hr. LC-MS showed 1 was consumed completely and one main peak with desired m/z was detected. After 12 hr, the reaction mixture was diluted with H₂O (2 mL), extracted by Ethyl acetate 6 mL (3 mL*2). Then organic phase was combined and washed by brine 10 mL, dried with Na₂SO₄, filtered. The organic phase was concentrated under reduced pressure at 40° C. to give a residue. The residue was purified by prep-HPLC (column: Waters Xbridge BEH C18 100*30 mm*10 um; mobile phase: [H₂O (10 mM NH₄HCO₃)-ACN]; gradient: 60%-90% B over 8.0 min). 2 (35 mg, 61.29 μmol, 32.46% yield, 98% purity) was obtained as a white solid.

LCMS: RT=1.551 min, MS cal.: 559.26, 560.26, [M+H]⁺=560.2

¹H NMR (400 MHz, CHCl3-d) δ=7.89 (d, J=10.0 Hz, 1H), 7.82 (s, 1H), 7.70 (d, J=3.6 Hz, 2H), 7.64 (t, J=8.0 Hz, 1H), 7.25 (s, 1H), 6.88 (d, J=7.2 Hz, 1H), 6.72 (d, J=8.4 Hz, 1H), 5.45 (s, 2H), 3.90 (d, J=17.6 Hz, 6H), 3.83-3.79 (m, 2H), 2.91 (br d, J=11.6 Hz, 2H), 2.63-2.53 (m, 1H), 2.18 (br t, J=10.8 Hz, 2H), 1.79-1.59 (m, 4H), 1.41 (s, 3H).

### General Procedure for Preparation of Compound 295 (3):

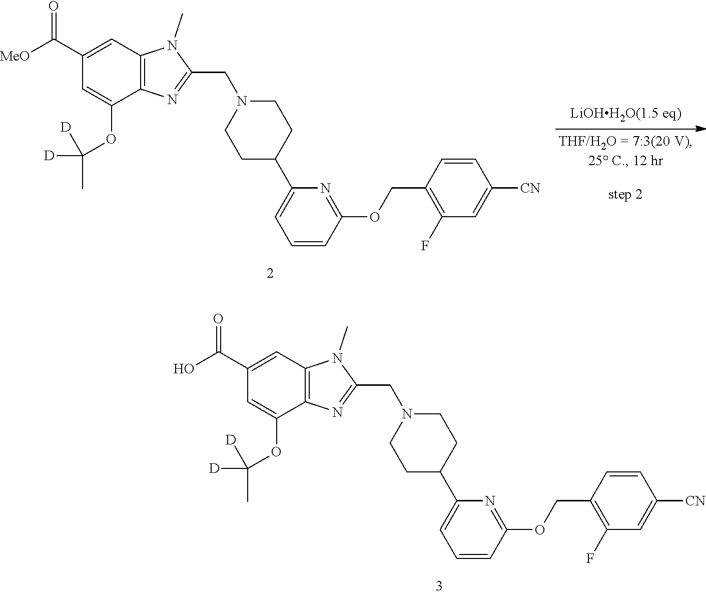 [SMILES: [2H]C([2H])(C)Oc1cc(C(=O)OC)cc2c1nc(CN1CCC(c3cccc(OCc4ccc(C#N)cc4F)n3)CC1)n2C ; [2H]C([2H])(C)Oc1cc(C(=O)O)cc2c1nc(CN1CCC(c3cccc(OCc4ccc(C#N)cc4F)n3)CC1)n2C]

To a solution of 2 (30 mg, 53.61 μmol, 1 eq) in THF (0.42 mL) was added LiOH·H₂O (3.37 mg, 80.41 μmol, 1.5 eq) and H₂O (0.18 mL). The mixture was stirred at 25° C. for 12 hr. The reaction was monitored by LCMS showed 2 was consumed completely and one main peak with desired m/z was detected. The residue was purified by prep-HPLC (column: Waters Xbridge BEH C18 100*30 mm*10 um; mobile phase: [H₂O (10 mM NH₄HCO₃)-ACN]; gradient: 20%-55% B over 8.0 min). 2-((4-(6-((4-Cyano-2-fluorobenzyl)oxy)pyridin-2-yl)piperidin-1-yl)methyl)-4-(ethoxy-1,1-d2)-1-methyl-1H-benzo[d]imidazole-6-carboxylic acid (24.98 mg, 44.87 μmol, 83.70% yield, 98% purity) was obtained as a white solid.

LCMS: RT=2.605 min, MS cal.: 545.24, 546.24, [M+H]⁺=546.3

HPLC: RT=10.281 min, purity: 98.38%

¹H NMR (400 MHz, DMSO-d₄) δ=7.89 (d, J=10.0 Hz, 1H), 7.78 (s, 1H), 7.70 (d, J=2.8 Hz, 2H), 7.67-7.62 (m, 1H), 7.25 (s, 1H), 6.88 (d, J=7.2 Hz, 1H), 6.72 (d, J=8.4 Hz, 1H), 5.46 (s, 2H), 3.91 (s, 3H), 3.81 (s, 2H), 2.91 (br d, J=10.8 Hz, 2H), 2.63-2.53 (m, 1H), 2.18 (br t, J=10.8 Hz, 2H), 1.80-1.59 (m, 4H), 1.41 (s, 3H)

### Example A51—Preparation of Compound 296. 2-((4-(6-((4-Chloro-2-fluorobenzyl)oxy)pyridin-2-yl)piperidin-1-yl)methyl)-4-(ethoxy-1,1-d2)-1-methyl-1H-benzo[d]imidazole-6-carboxylic acid (Compound 296) (3)

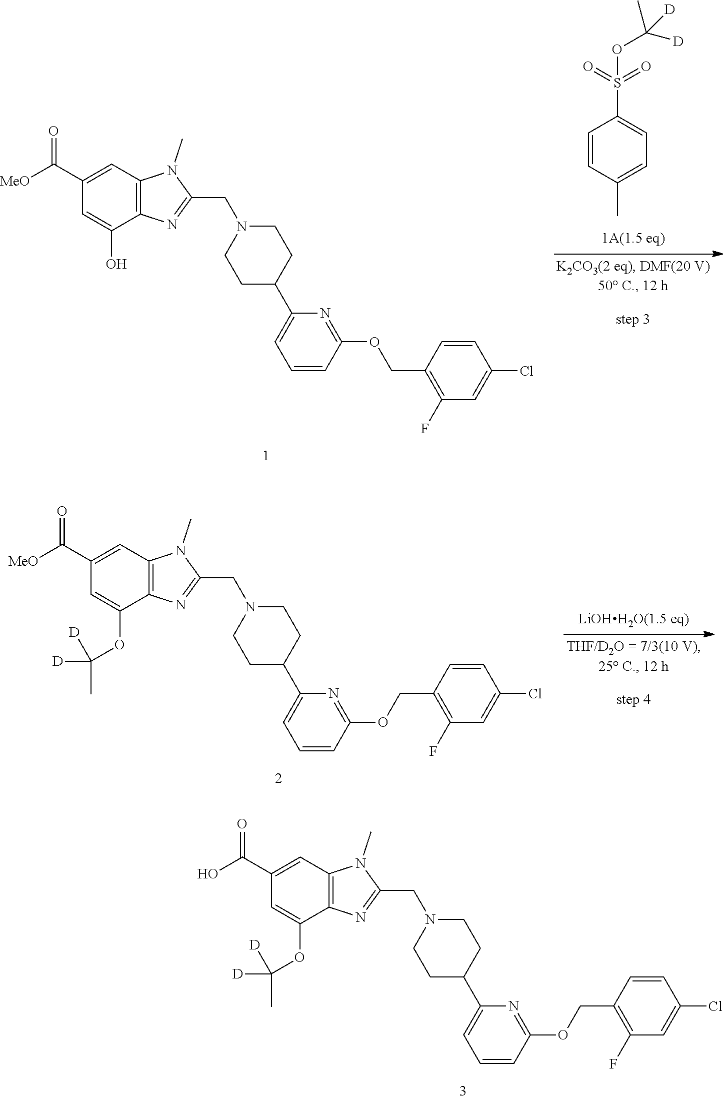 [SMILES: COC(=O)c1cc(O)c2nc(CN3CCC(c4cccc(OCc5ccc(Cl)cc5F)n4)CC3)n(C)c2c1 ; [2H]C([2H])(C)OS(=O)(=O)c1ccc(C)cc1 ; [2H]C([2H])(C)Oc1cc(C(=O)OC)cc2c1nc(CN1CCC(c3cccc(OCc4ccc(Cl)cc4F)n3)CC1)n2C ; [2H]C([2H])(C)Oc1cc(C(=O)O)cc2c1nc(CN1CCC(c3cccc(OCc4ccc(Cl)cc4F)n3)CC1)n2C]

### General Procedure for Preparation of Intermediate 2:

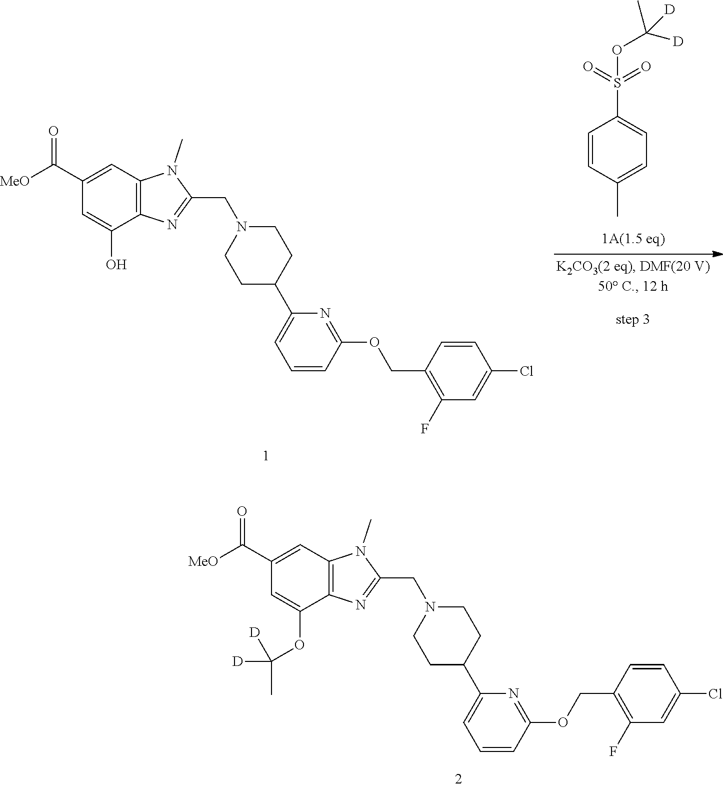 [SMILES: COC(=O)c1cc(O)c2nc(CN3CCC(c4cccc(OCc5ccc(Cl)cc5F)n4)CC3)n(C)c2c1 ; [2H]C([2H])(C)OS(=O)(=O)c1ccc(C)cc1 ; [2H]C([2H])(C)Oc1cc(C(=O)OC)cc2c1nc(CN1CCC(c3cccc(OCc4ccc(Cl)cc4F)n3)CC1)n2C]

A mixture of 1 (100 mg, 185.53 μmol, 1 eq), 1A (56.29 mg, 278.29 μmol, 1.5 eq), K₂CO₃ (51.28 mg, 371.06 μmol, 2 eq), in DMF (1 mL) was degassed and purged with N₂ for 3 times, and then the mixture was stirred at 50° C. for 12 hr under N₂ atmosphere. LC-MS showed 1 was consumed completely and one main peak with desired mass was detected. The reaction mixture was diluted with H₂O 10 mL and extracted with EtOAc 30 mL (10 mL*3). The combined organic layers were washed with brine 10 mL dried over Na₂SO₄, filtered, and concentrated under reduced pressure to give a residue. The residue was purified by prep-HPLC (neutral condition column: Waters Xbridge Prep OBD C18 150*40 mm*10 um; mobile phase: [H₂O (10 mM NH₄HCO₃)-ACN]; gradient: 65%-95% B over 8.0 min). 2 (70 mg, 123.01 μmol, 66.30% yield) was obtained as a white solid.

LCMS: Rt=1.685 min, MS cal.: 568.2, [M+H]⁺=569.2

1H NMR (400 MHz, DMSO-d6)=7.82 (d, J=1.20 Hz, 1H) 7.66-7.60 (m, 1H) 7.56 (t, J=8.16 Hz, 1H) 7.46 (dd, J=9.88, 2.03 Hz, 1H) 7.29 (dd, J=8.28, 1.85 Hz, 1H) 7.25 (d, J=1.30 Hz, 1H) 6.87 (d, J=7.36 Hz, 1H) 6.67 (d, J=8.12 Hz, 1H) 5.36 (s, 2H) 3.93 (s, 3H) 3.88 (s, 3H) 3.82 (s, 2H) 2.93 (br d, J=11.20 Hz, 2H) 2.59 (ddt, J=15.36, 11.68, 3.52, 3.52 Hz, 1H) 2.25-2.14 (m, 2H) 1.84-1.73 (m, 2H) 1.72-1.62 (m, 2H) 1.41 (s, 3H)

### General Procedure for Preparation of Compound 296 (3):

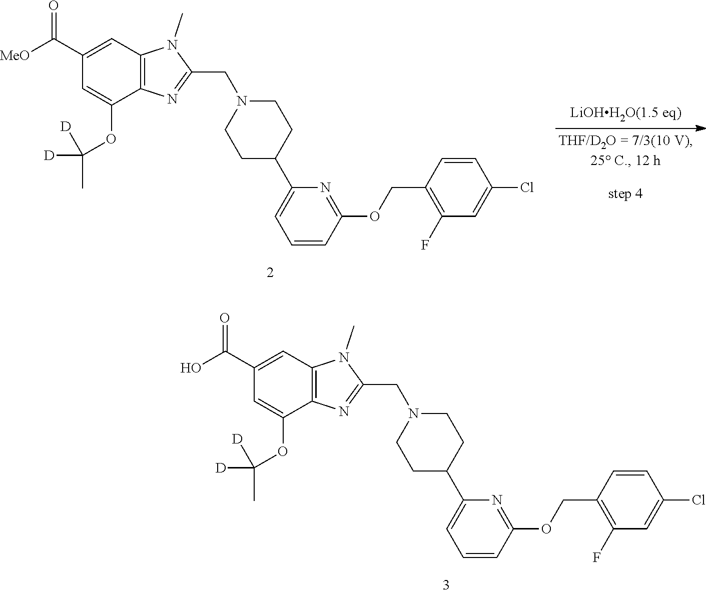 [SMILES: [2H]C([2H])(C)Oc1cc(C(=O)OC)cc2c1nc(CN1CCC(c3cccc(OCc4ccc(Cl)cc4F)n3)CC1)n2C ; [2H]C([2H])(C)Oc1cc(C(=O)O)cc2c1nc(CN1CCC(c3cccc(OCc4ccc(Cl)cc4F)n3)CC1)n2C]

A mixture of 2 (90 mg, 158.15 μmol, 1 eq), LiOH·H₂O (9.96 mg, 237.23 μmol, 1.5 eq) in THF (0.7 mL) D₂O (0.3 mL) was degassed and purged with N₂ for 3 times, and then the mixture was stirred at 25° C. for 12 hr under N₂ atmosphere. LC-MS (ET43536-1741-P1B1 RT=1.182 min) showed 2 was consumed completely and one main peak with desired mass was detected. The residue was purified directly. The residue was purified by prep-HPLC (neutral condition column: Waters Xbridge Prep OBD C18 150*40 mm*10 um; mobile phase: [H₂O (10 mM NH₄HCO₃)-ACN]; gradient: 20%-70% B over 8.0 min). 2-((4-(6-((4-Chloro-2-fluorobenzyl)oxy)pyridin-2-yl)piperidin-1-yl)methyl)-4-(ethoxy-1,1-d2)-1-methyl-1H-benzo[d]imidazole-6-carboxylic acid (24.26 mg, 43.58 μmol, 27.55% yield, 99.70% purity) was obtained as a white solid.

LCMS: Rt₁=1.182 min, MS cal.: 554.2, [M+H]⁺=555.1

1H NMR (400 MHz, DMSO-d6) δ=7.76 (d, J=1.20 Hz, 1H) 7.65-7.59 (m, 1H) 7.55 (t, J=8.16 Hz, 1H) 7.45 (dd, J=10.00, 2.03 Hz, 1H) 7.28 (dd, J=8.24, 1.79 Hz, 1H) 7.25 (d, J=1.32 Hz, 1H) 6.86 (d, J=7.2 Hz, 1H) 6.66 (d, J=8.12 Hz, 1H) 5.36 (s, 2H) 3.90 (s, 3H) 3.80 (s, 2H) 2.92 (br d, J=11.2 Hz, 2H) 2.59 (tt, J=11.4, 3.6 Hz, 1H) 2.25-2.13 (m, 2H) 1.83-1.63 (m, 2H) 1.72-1.62 (m, 1H) 1.40 (s, 3H)

### Example A52—Preparation of Compound 316. 5-(2-((4-(6-((4-Chloro-2-fluorobenzyl)oxy)pyridin-2-yl)piperidin-1-yl)methyl)-4-(difluoromethoxy)-1-methyl-1H-benzo[d]imidazol-6-yl) isoxazol-3-ol (Compound 316) (7)

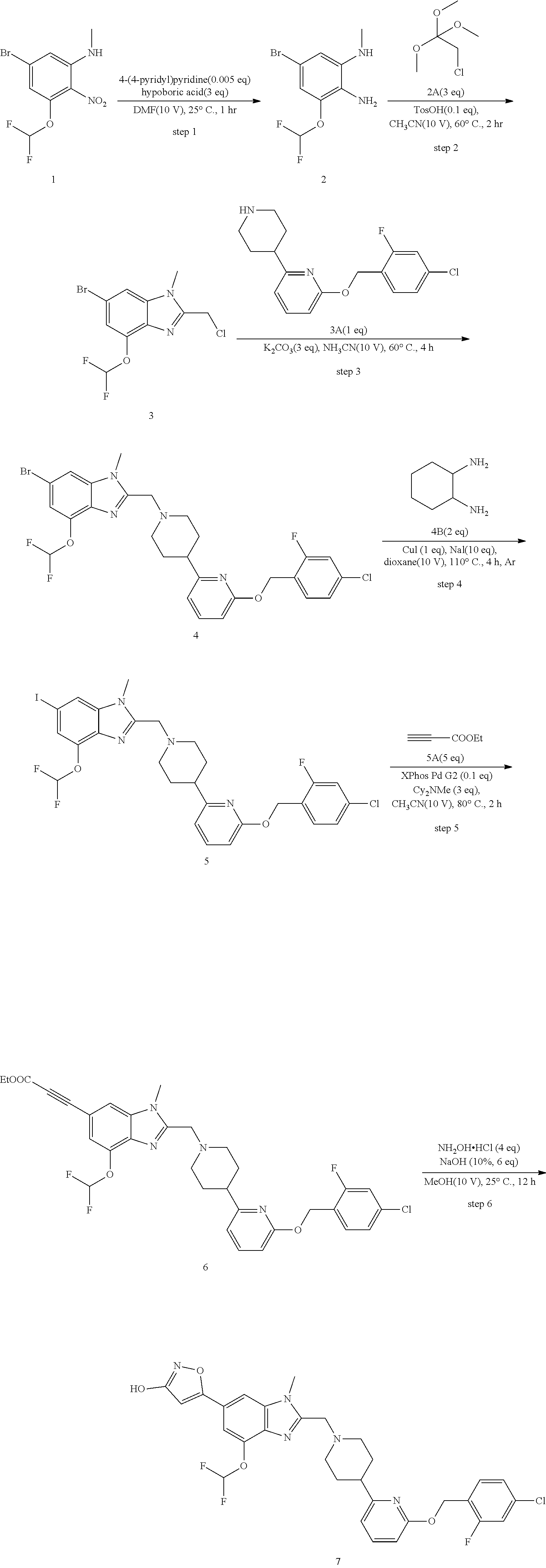

General procedure for preparation of Intermediate 2:

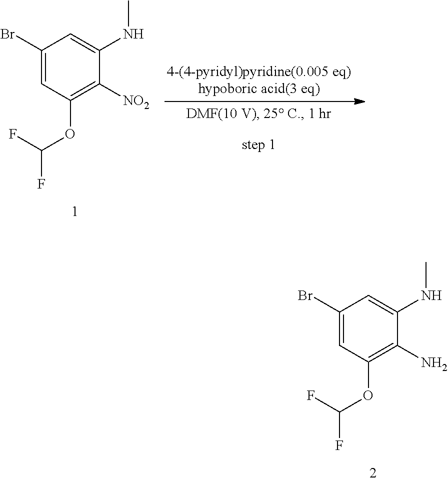 [SMILES: CNc1cc(Br)cc(OC(F)F)c1[N+](=O)[O-] ; CNc1cc(Br)cc(OC(F)F)c1N]

To a solution of 1 (5.4 g, 18.18 mmol, 1 eq) in DMF (54 mL) was added 4-(4-pyridyl)pyridine (14.20 mg, 90.89 μmol, 0.005 eq) and hypoboric acid (4.89 g, 54.54 mmol, 3 eq). The mixture was stirred at 25° C. for 1 hr. LC-MS showed 1 was consumed completely and desired mass was detected. The residue was diluted with H₂O (30 mL) and extracted with EtOAc (90 mL). The combined organic layers were washed with brine (30 mL), dried over Na₂SO₄, filtered, and concentrated under reduced pressure to give a residue. The residue was purified by column chromatography (SiO₂, Petroleum ether/Ethyl acetate=5/1 to 1/1). 2 (3.1 g, 11.61 mmol, 63.85% yield) was obtained as a black oil.

LCMS: RT=1.239 min, MS cal.: 265.99 [M+H]⁺=451.0

¹H NMR (400 MHz, CHCl3-d) δ=6.72 (s, 1H), 6.64 (d, J=2.0 Hz, 1H), 6.46 (t, J=74.0 Hz, 1H), 3.76-3.29 (m, 3H), 2.87 (s, 3H)

### General Procedure for Preparation of Intermediate 3:

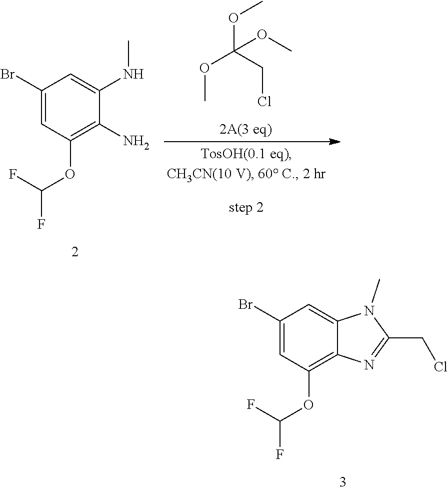

To a solution of 2 (3 g, 11.23 mmol, 1 eq), 2A (5.21 g, 33.70 mmol, 4.54 mL, 3 eq) in CH₃CN (30 mL) was added TosOH (193.43 mg, 1.12 mmol, 0.1 eq). The mixture was stirred at 60° C. for 2 hr. LC-MS showed 2 was consumed completely and desired mass was detected. The reaction mixture was added with aq. Na₂CO₃ to adjust pH-8-9. The mixture was diluted with H₂O (20 mL) and extracted with EtOAc (30 mL). The combined organic layers were washed with brine (20 mL), dried over Na₂SO₄ filtered and concentrated under reduced pressure to give a residue. The residue was purified by column chromatography (SiO₂, Petroleum ether/Ethyl acetate=5/1 to 1/1). 3 (3.315 g, 10.18 mmol, 90.65% yield) was obtained as a purple solid.

LCMS: RT=0.503 min, MS cal.: 325.54, [M+H]⁺=326.9

¹H NMR (400 MHz, CHCl3-d) δ=7.35 (t, J=74.0 Hz, 1H), 7.35 (d, J=1.2 Hz, 1H), 7.18 (d, J=1.2 Hz, 1H), 4.82 (s, 2H), 3.85 (s, 3H)

### General Procedure for Preparation of Intermediate 4:

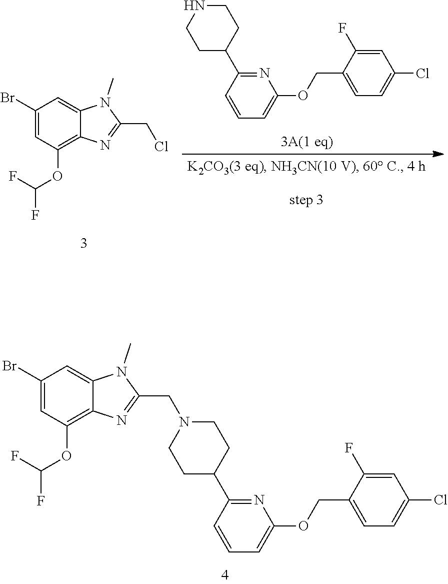 [SMILES: Cn1c(CCl)nc2c(OC(F)F)cc(Br)cc21 ; Fc1cc(Cl)ccc1COc1cccc(C2CCNCC2)n1 ; Cn1c(CN2CCC(c3cccc(OCc4ccc(Cl)cc4F)n3)CC2)nc2c(OC(F)F)cc(Br)cc21]

To a solution of 3 (0.5 g, 1.54 mmol, 1 eq), 3A (492.71 mg, 1.54 mmol, 1 eq) in CH₃CN (10 mL) was added K₂CO₃ (636.82 mg, 4.61 mmol, 3 eq). The mixture was stirred at 60° C. for 4 hr. LC-MS showed 3 was consumed completely and desired mass was detected. The residue was diluted with H₂O (20 mL) and extracted with EtOAc (30 mL). The combined organic layers were washed with brine (20 mL), dried over Na₂SO₄ filtered and concentrated under reduced pressure to give a residue. The residue was purified by column chromatography (SiO₂, Petroleum ether/Ethyl acetate=5/1 to 2/1). 4 (1.6 g, 2.62 mmol, 85.41% yield) was obtained as a red solid.

LCMS: RT=1.830 min, MS cal.: 609.86, [M+H]⁺=611.0

LCMS: RT=1.829 min, MS cal.: 609.86, [M+H]⁺=611.0

¹H NMR (400 MHz, DMSO-d₆) δ=7.76 (d, J=1.6 Hz, 1H), 7.68 (t, J=74.4 Hz, 1H), 7.65-7.58 (m, 1H), 7.54 (t, J=8.4 Hz, 1H), 7.43 (dd, J=2.0, 10.0 Hz, 1H), 7.27 (dd, J=1.6, 8.4 Hz, 1H), 7.15 (d, J=1.2 Hz, 1H), 6.85 (d, J=7.2 Hz, 1H), 6.66 (d, J=8.0 Hz, 1H), 5.35 (s, 2H), 3.89 (s, 3H), 3.80 (s, 2H), 2.91 (br d, J=11.2 Hz, 2H), 2.57 (tt, J=4.0, 11.6 Hz, 1H), 2.24-2.14 (m, 2H), 1.82-1.61 (m, 4H)

### General Procedure for Preparation of Intermediate 4& Intermediate 5:

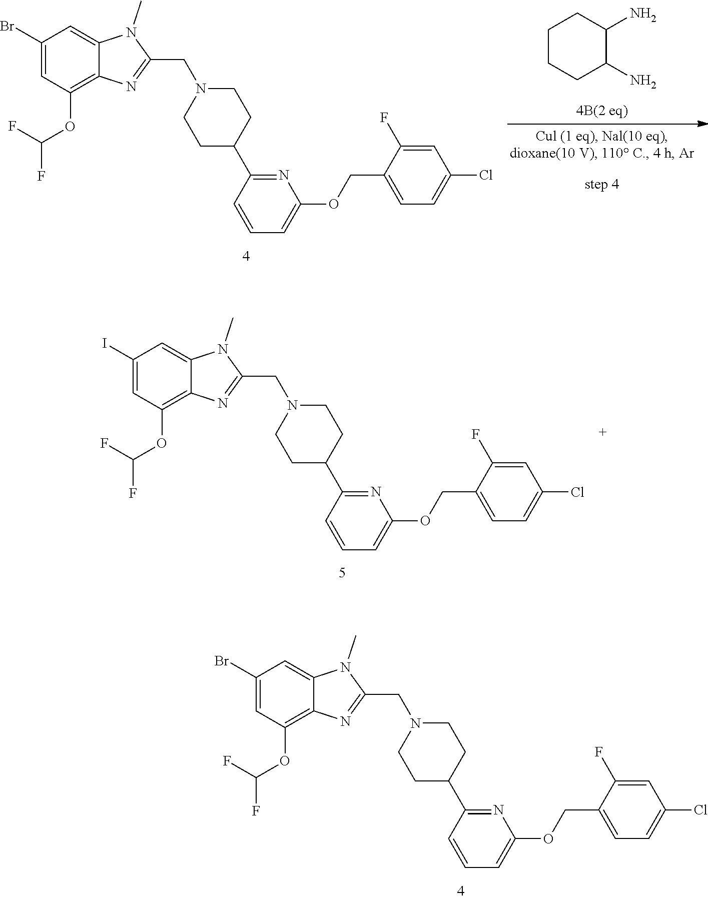 [SMILES: Cn1c(CN2CCC(c3cccc(OCc4ccc(Cl)cc4F)n3)CC2)nc2c(OC(F)F)cc(Br)cc21 ; NC1CCCCC1N ; Cn1c(CN2CCC(c3cccc(OCc4ccc(Cl)cc4F)n3)CC2)nc2c(OC(F)F)cc(I)cc21 ; Cn1c(CN2CCC(c3cccc(OCc4ccc(Cl)cc4F)n3)CC2)nc2c(OC(F)F)cc(Br)cc21]

To a solution of 4 (200 mg, 327.94 μmol, 1 eq) in dioxane (2 mL) was added CuI (62.46 mg, 327.94 μmol, 1 eq), NaI (491.55 mg, 3.28 mmol, 10 eq), 4B (74.90 mg, 655.88 μmol, 80.45 μL, 2 eq). The mixture was stirred at 110° C. for 4 hr. LC-MS showed ˜50% of 4 remained. One new peak was shown on LC-MS and ˜50% of desired Compound was detected. Three batches crude product mixture was combined for workup. The reaction mixture was filtered through celite pad. The filtrate was diluted with H₂O (20 mL) and extracted with EtOAc (30 mL). The combined organic layers were washed with brine (20 mL), dried over Na₂SO₄, filtered, and concentrated under reduced pressure to give a residue. The residue was purified by column chromatography (SiO₂, Petroleum ether/Ethyl acetate=3/1 to 1/1). 4& 5 (600 mg crude in total) as a white solid.

LCMS: RT=1.850 min, MS cal.: 656.86, [M+H]⁺=657.0

### General Procedure for Preparation of Intermediate 4:

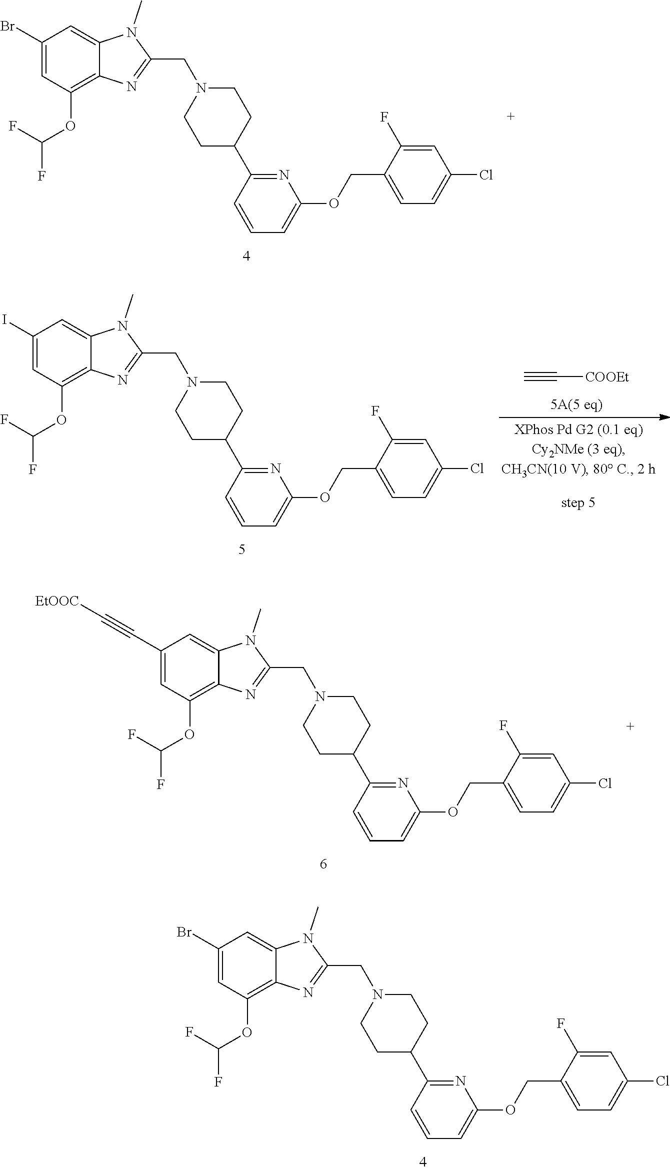

To a solution of 4&5 (600 mg, crude), 5A (224.02 mg, 2.28 mmol, 224.02 μL, 5 eq) in CH₃CN (3 mL) was added Cy₂NMe (267.64 mg, 1.37 mmol, 290.60 μL, 3 eq) and XPhos Pd G2 (35.93 mg, 45.67 μmol, 0.1 eq). The mixture was stirred at 80° C. for 2 hr. LCMS showed 5 was consumed completely and desired mass was detected. The mixture was diluted with H₂O (20 mL) and extracted with EtOAc (30 mL). The combined organic layers were washed with brine (20 mL), dried over Na₂SO₄ filtered and concentrated under reduced pressure to give a residue. The residue was purified by prep-HPLC (neutral condition; column: Waters Xbridge BEH C18 100*30 mm*10 um; mobile phase: [H₂O (10 mM NH₄HCO₃)-ACN]; gradient: 75%-95% B over 8.0 min). 6 (140 mg, 223.27 μmol, 48.89% yield) was obtained as a white solid. 4 (360 mg, 590.30 μmol, 64.62% yield) was obtained as a white solid.

LCMS: RT=1.834 min, MS cal.: 627.05, [M+H]⁺=627.2

¹H NMR (400 MHz, DMSO-d₆) δ=7.96 (d, J=1.2 Hz, 1H), 7.66 (t, J=74.4 Hz, 1H), 7.62 (t, J=7.6 Hz, 1H), 7.55 (t, J=8.0 Hz, 1H), 7.44 (dd, J=2.0, 10.0 Hz, 1H), 7.28 (dd, J=1.6, 8.4 Hz, 1H), 7.24 (s, 1H), 6.86 (d, J=7.2 Hz, 1H), 6.66 (d, J=8.0 Hz, 1H), 5.36 (s, 2H), 4.25 (q, J=7.2 Hz, 2H), 3.94 (s, 3H), 3.85 (s, 2H), 2.93 (br d, J=11.2 Hz, 2H), 2.62-2.55 (m, 1H), 2.22 (br t, J=10.8 Hz, 2H), 1.83-1.63 (m, 5H), 1.28 (t, J=7.2 Hz, 3H)

### General Procedure for Preparation of Compound 316 (7):

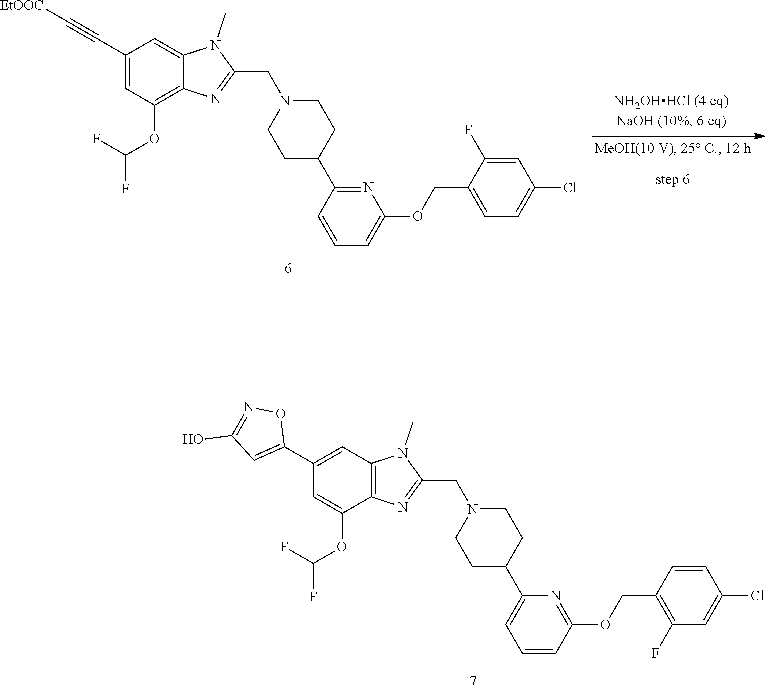 [SMILES: CCOC(=O)C#Cc1cc(OC(F)F)c2nc(CN3CCC(c4cccc(OCc5ccc(Cl)cc5F)n4)CC3)n(C)c2c1 ; Cn1c(CN2CCC(c3cccc(OCc4ccc(Cl)cc4F)n3)CC2)nc2c(OC(F)F)cc(-c3cc(O)no3)cc21]

To a solution of 6 (100 mg, 159.48 μmol, 1 eq) in MeOH (1 mL) was added NaOH (382.72 mg, 956.86 μmol, 10% purity, 6 eq) and NH₂OH HCl (44.33 mg, 637.91 μmol, 4 eq). The mixture was stirred at 25° C. for 12 hr. LCMS showed 6 was consumed completely and desired mass was detected. The reaction mixture was purified directly. The residue was purified by prep-HPLC (neutral condition; column: column: Waters Xbridge Prep OBD C18 150*40 mm*10 um; mobile phase: [H₂O (10 mM NH₄HCO₃)-ACN]; gradient: 35%-55% B over 8.0 min). 5-(2-((4-(6-((4-Chloro-2-fluorobenzyl)oxy)pyridin-2-yl)piperidin-1-yl)methyl)-4-(difluoromethoxy)-1-methyl-1H-benzo[d]imidazol-6-yl) isoxazol-3-ol (29.92 mg, 48.73 μmol, 30.56% yield) was obtained as a white solid.

LCMS: RT=3.048 min, MS cal.: 614.01, [M+H]⁺=614.2

HPLC: RT=12.568 min, purity: 99.8%

¹H NMR (400 MHz, MeOH-d4) δ=7.85 (d, J=1.2 Hz, 1H), 7.56 (t, J=7.6 Hz, 1H), 7.51-7.45 (m, 1H), 7.40 (s, 1H), 7.31 (t, J=74.4 Hz, 1H), 7.21-7.14 (m, 2H), 6.81 (d, J=7.2 Hz, 1H), 6.62 (d, J=8.0 Hz, 1H), 6.39 (s, 1H), 5.39 (s, 2H), 4.02 (s, 3H), 3.91 (s, 2H), 3.03 (br d, J=11.2 Hz, 2H), 2.69-2.58 (m, 1H), 2.38-2.27 (m, 2H), 1.90-1.81 (m, 4H)

### Example A53—Preparation of Compound 317. 2-((4-(6-((4-Cyano-2-fluorobenzyl)oxy)pyridin-2-yl)piperidin-1-yl)methyl)-4-((1r,3r)-3-methoxycyclobutoxy)-1-methyl-1H-benzo[d]imidazole-6-carboxylic acid (Compound 317) (3)

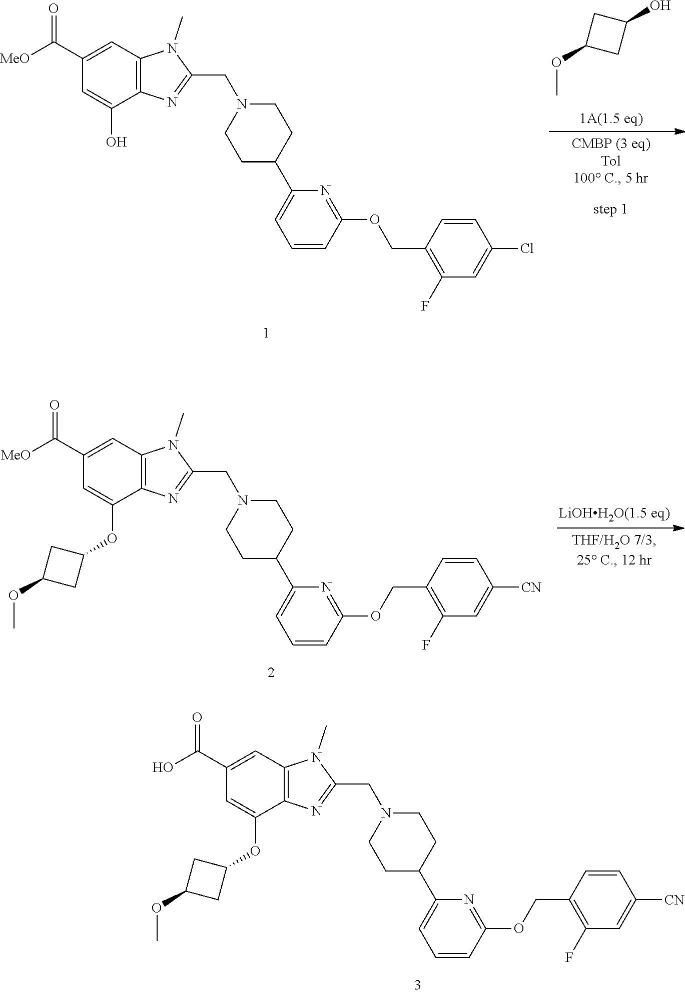

### General Procedure for Preparation of Intermediate 2:

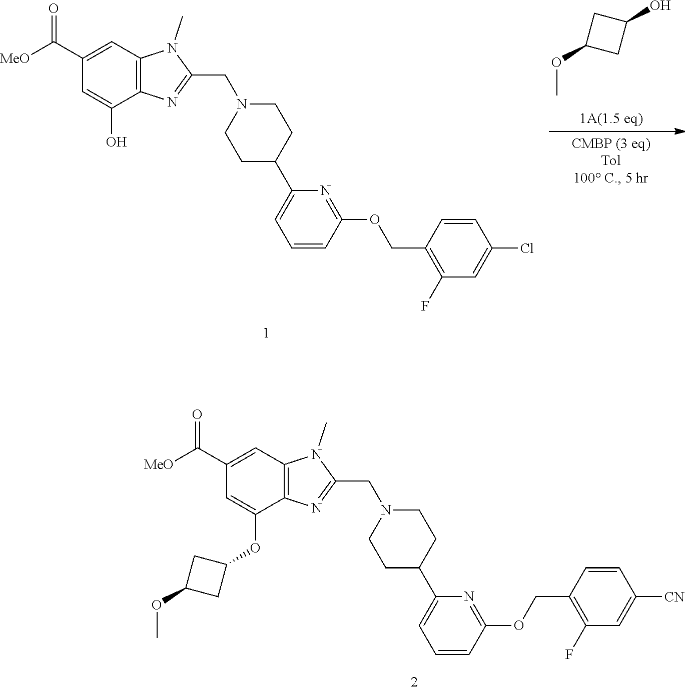

A mixture of 1 (100 mg, 188.84 μmol, 1 eq), 1A (28.93 mg, 283.25 μmol, 1.5 eq), CMBP (136.73 mg, 566.51 μmol, 3 eq) in Toluene (2 mL) was degassed and purged with N₂ for 3 times, and then the mixture was stirred at 100° C. for 5 hr under N₂ atmosphere. LC-MS (RT=1.519 min) showed 1 was consumed completely and one main peak with desired mass was detected. The reaction mixture was diluted with H₂O 20 mL and extracted with EtOAc 60 mL (20 mL*3). The combined organic layers were washed with brine 20 mL, dried over Na₂SO₄, filtered, and concentrated under reduced pressure to give a residue. The residue was purified by column chromatography (SiO₂, Petroleum ether/Ethyl acetate=10/1 to 1/1). 2 (58 mg, 94.51 μmol, 50.05% yield) was obtained as a yellow solid.

LCMS: RT=1.674 min, MS cal.: 611.20, [M+H]⁺=612.4

SFC: ee %=86.26%

¹H NMR (400 MHz, CHCl3-d) δ=7.84-7.75 (m, 1H), 7.68-7.58 (m, 1H), 7.57-7.49 (m, 1H), 7.48-7.42 (m, 1H), 7.37 (d, J=9.2 Hz, 1H), 7.20 (br s, 1H), 6.82-6.70 (m, 1H), 6.65 (br d, J=7.2 Hz, 1H), 5.50 (s, 2H), 5.21-5.08 (m, 1H), 4.25-4.13 (m, 2H), 4.07-3.86 (m, 7H), 3.30 (s, 3H), 3.02-2.89 (m, 2H), 2.70-2.51 (m, 5H), 2.36-2.16 (m, 2H), 1.93-1.81 (m, 2H), 1.79-1.72 (m, 1H), 1.63-1.50 (m, 2H)

### General Procedure for Preparation of Compound 317 (3):

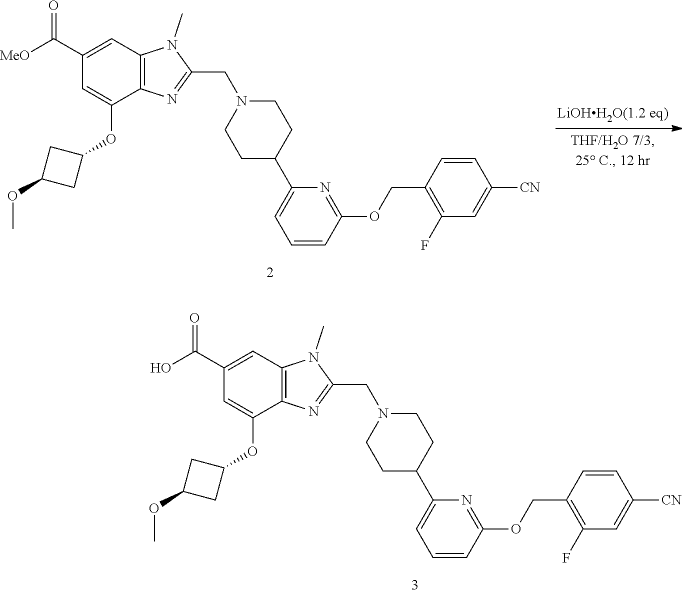 [SMILES: COC(=O)c1cc(O[C@H]2C[C@H](OC)C2)c2nc(CN3CCC(c4cccc(OCc5ccc(C#N)cc5F)n4)CC3)n(C)c2c1 ; CO[C@H]1C[C@H](Oc2cc(C(=O)O)cc3c2nc(CN2CCC(c4cccc(OCc5ccc(C#N)cc5F)n4)CC2)n3C)C1]

A mixture of 2 (45 mg, 73.33 μmol, 1 eq), LiOH·H₂O (3.69 mg, 87.99 μmol, 1.2 eq) in THF (0.7 mL) and H₂O (0.3 mL) was degassed and purged with N₂ for 3 times, and then the mixture was stirred at 25° C. for 12 hr under N₂ atmosphere. LC-MS (ET83888-171, product, RT=1.087 min) showed 2 was consumed completely and one main peak with desired mass was detected. The reaction was purified directly. The residue was purified by prep-HPLC (column: Waters Xbridge Prep OBD C18 150*40 mm*10 um; mobile phase: [H₂O (10 mM NH₄HCO₃)-ACN]; gradient: 25%-65% B over 8.0 min). 2-((4-(6-((4-Cyano-2-fluorobenzyl)oxy)pyridin-2-yl)piperidin-1-yl)methyl)-4-((1r,3r)-3-methoxycyclobutoxy)-1-methyl-1H-benzo[d]imidazole-6-carboxylic acid (20.97 mg, 34.65 μmol, 47.26% yield, 99.09% purity) was obtained as a white solid.

LCMS: RT=2.308 min, MS cal.: 599.25, [M+1]⁺=600.2

HPLC: RT=10.38 min, purity: 99.09%

SFC: ee %=100%

¹H NMR (400 MHz, DMSO) δ=7.86 (s, 1H), 7.66 (t, J=7.2 Hz, 1H), 7.62-7.49 (m, 3H), 7.23 (s, 1H), 6.83 (d, J=7.2 Hz, 1H), 6.67 (d, J=8.4 Hz, 1H), 5.50 (s, 2H), 5.08 (m, J=5.2 Hz, 1H), 4.26-4.17 (m, 1H), 3.98 (s, 3H), 3.94 (s, 2H), 3.29 (s, 3H), 3.06 (br d, J=11.2 Hz, 2H), 2.64 (br t, J=7.6 Hz, 1H), 2.55 (t, J=5.6 Hz, 4H), 2.43-2.31 (m, 2H), 1.90-1.79 (m, 4H)

### Example A54—Preparation of Compound 318. 2-((4-(6-((4-Cyano-2-fluorobenzyl)oxy)pyridin-2-yl)piperidin-1-yl)methyl)-4-((1s,3s)-3-methoxycyclobutoxy)-1-methyl-1H-benzo[d]imidazole-6-carboxylic acid (Compound 318) (3)

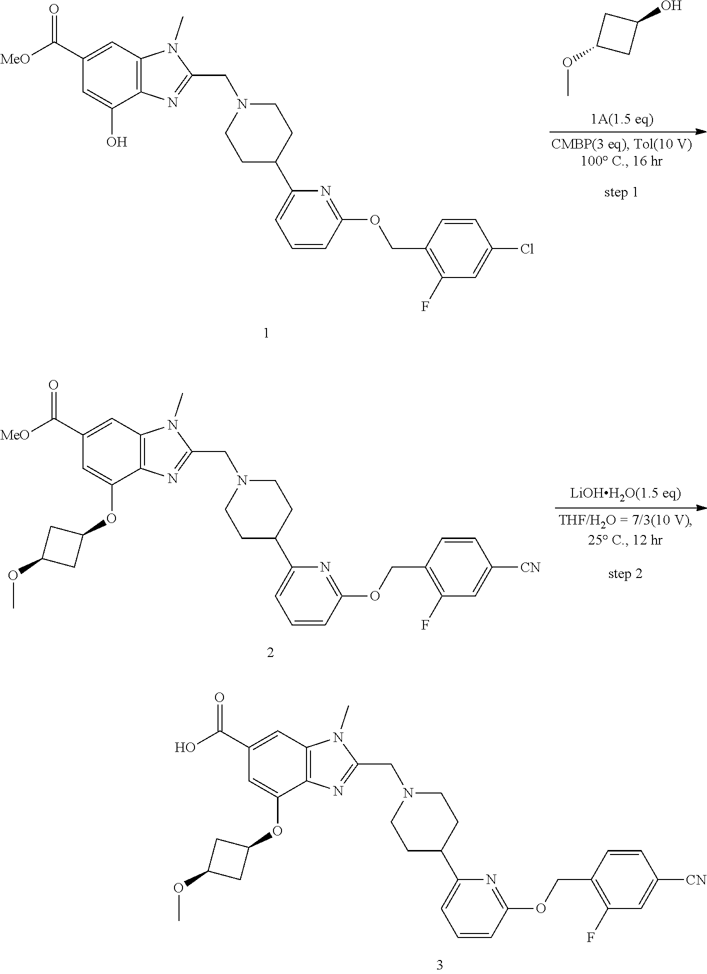

### General Procedure for Preparation of Intermediate 2:

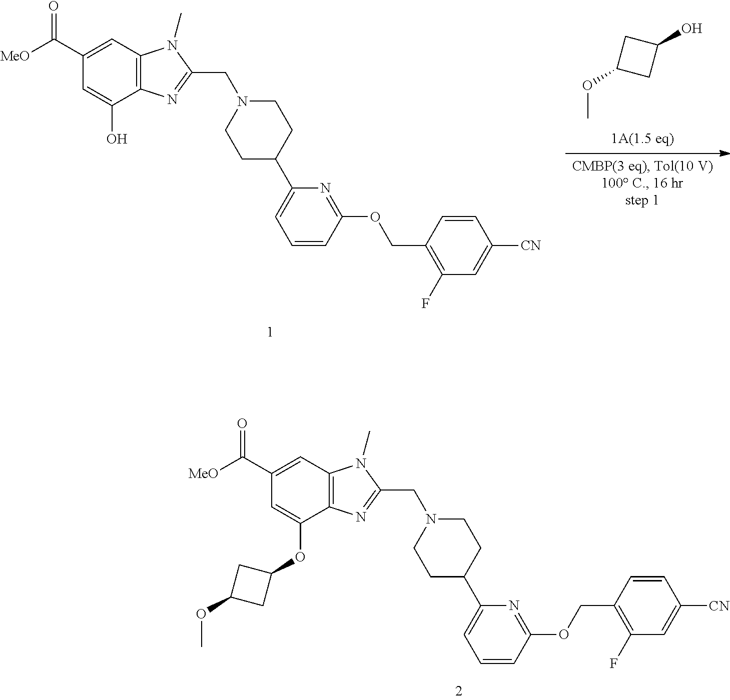

A mixture of 1 (100 mg, 188.84 μmol, 1 eq), 1A (136.73 mg, 566.51 μmol, 3 eq) in Toluene (1 mL) was degassed and purged with N₂ for 3 times, and then the mixture was stirred at 100° C. for 16 hr under N₂ atmosphere. LC-MS showed 1 was consumed completely and one main peak with desired mass was detected. The mixture was diluted with H₂O (30 mL), the mixture was extracted by EtOAc (20 mL*3). Then organic phase was combined and washed by H₂O (20 mL), aq. brine (20 mL), dried with Na₂SO₄, filtered. The organic phase was concentrated under reduced pressure at 45° C. to give a residue. The residue was purified by column chromatography (SiO₂, PE/EtOAc=10/1 to 0/1). 2 (135 mg, crude) was obtained as a brown oil.

LCMS: Rt=0.447 min, MS cal.: 613.27/614.27, [M+H]⁺=614.3/615.2

¹HNMR (400 MHz, CHCl3-d) δ=7.13 (br t, J=7.2 Hz, 1H), 7.03 (t, J=8.0 Hz, 1H), 6.94 (br d, J=8.0 Hz, 1H), 6.87 (d, J=9.2 Hz, 1H), 6.78-6.75 (m, 3H), 6.26 (d, J=7.2 Hz, 1H), 6.15 (d, J=8.4 Hz, 1H), 5.00 (s, 2H), 4.81 (s, 2H), 4.13-4.06 (m, 1H), 3.66-3.60 (m, 1H), 3.49-3.44 (m, 5H), 3.43-3.36 (m, 1H), 3.26-3.19 (m, 1H), 2.78 (s, 3H), 2.54-2.45 (m, 3H), 2.09 (br d, J=10.8 Hz, 1H), 1.89-1.75 (m, 3H), 1.42-1.11 (m, 4H), 0.77 (t, J=7.2 Hz, 2H)

### General Procedure for Preparation of Compound 318 (3):

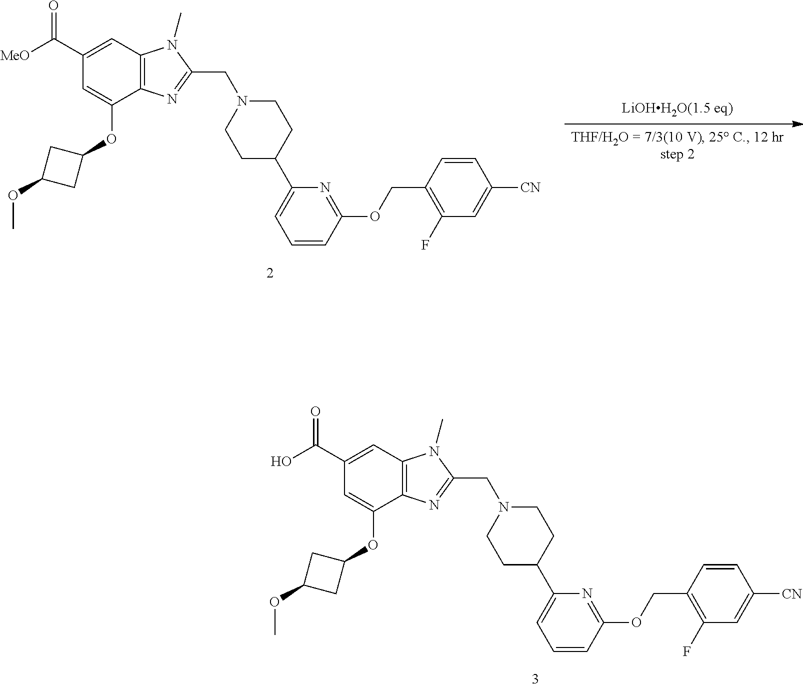

To a solution of 2 (130 mg, 211.84 μmol, 1 eq) in THF (0.7 mL) and H₂O (0.3 mL) was added LiOH·H₂O (13.33 mg, 317.76 μmol, 1.5 eq). The mixture was stirred at 25° C. for 12 hr. LC-MS (ET77627-430-P1A1 Rt=1.066 min) showed 2 was consumed completely and one main peak with desired mass was detected. The reaction was filtered directly. The residue was purified by prep-HPLC (column: Waters Xbridge BEH C18 100*25 mm*10 um; mobile phase: [H₂O (10 mM NH₄HCO₃)-ACN]; gradient: 20%-55% B over 8.0 min). 2-((4-(6-((4-Cyano-2-fluorobenzyl)oxy)pyridin-2-yl)piperidin-1-yl)methyl)-4-((1s,3s)-3-methoxycyclobutoxy)-1-methyl-1H-benzo[d]imidazole-6-carboxylic acid (27.27 mg, 45.48 μmol, 21.47% yield) was obtained as a white solid

HPLC: Rt=10.344 min, purity 94.88%

LCMS: Rt=2.584 min, MS cal.: 599.25/600.25, [M+H]⁺=600.3/601.3

¹H NMR (400 MHz, DMSO-d₆) δ=7.88 (br d, J=9.6 Hz, 1H), 7.78 (s, 1H), 7.73-7.61 (m, 3H), 7.14 (s, 1H), 6.88 (d, J=7.8 Hz, 1H), 6.71 (d, J=8.4 Hz, 1H), 5.45 (s, 2H), 4.64 (br d, J=7.2 Hz, 1H), 3.90 (s, 3H), 3.80 (s, 2H), 3.71 (br d, J=7.2 Hz, 1H), 3.21-3.14 (m, 3H), 2.96-2.85 (m, 4H), 2.61-2.55 (m, 1H), 2.23-2.12 (m, 2H), 2.05-1.94 (m, 2H), 1.80-1.59 (m, 4H).

### Example A55—Preparation of Compound 319. 2-((4-(6-((4-Cyano-2-fluorobenzyl)oxy)pyridin-2-yl)piperidin-1-yl)methyl)-1-methyl-4-propoxy-1H-benzo[d]imidazole-6-carboxylic acid (Compound 319) (3)

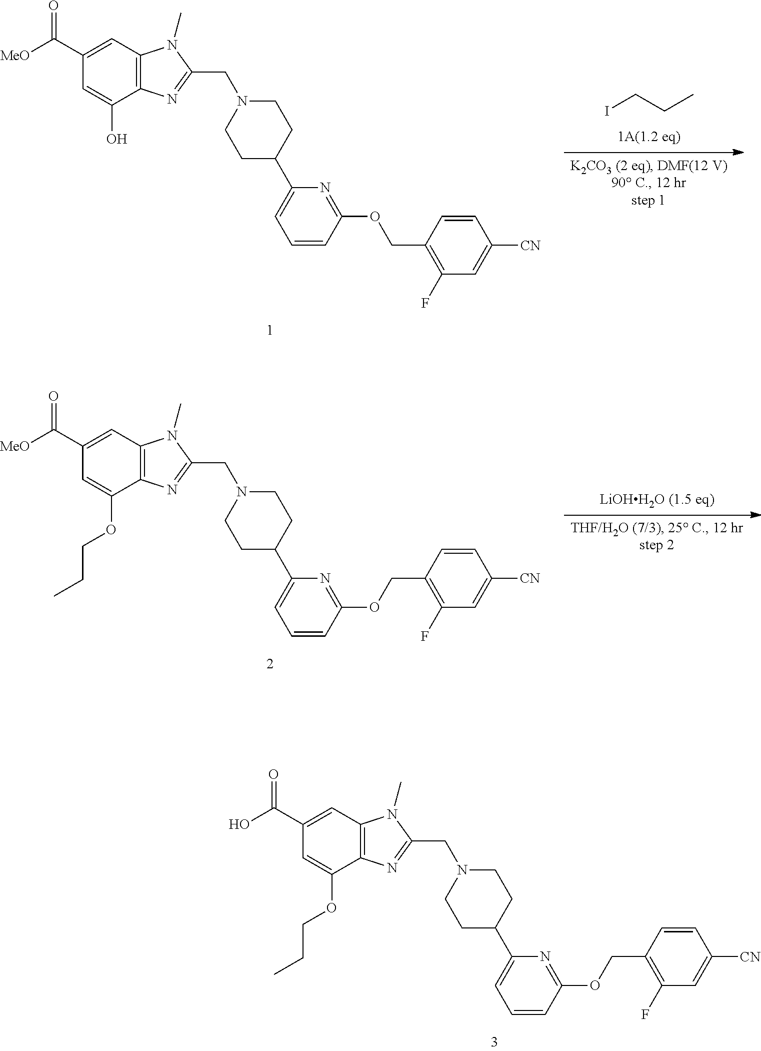

### General Procedure for Preparation of Intermediate 2:

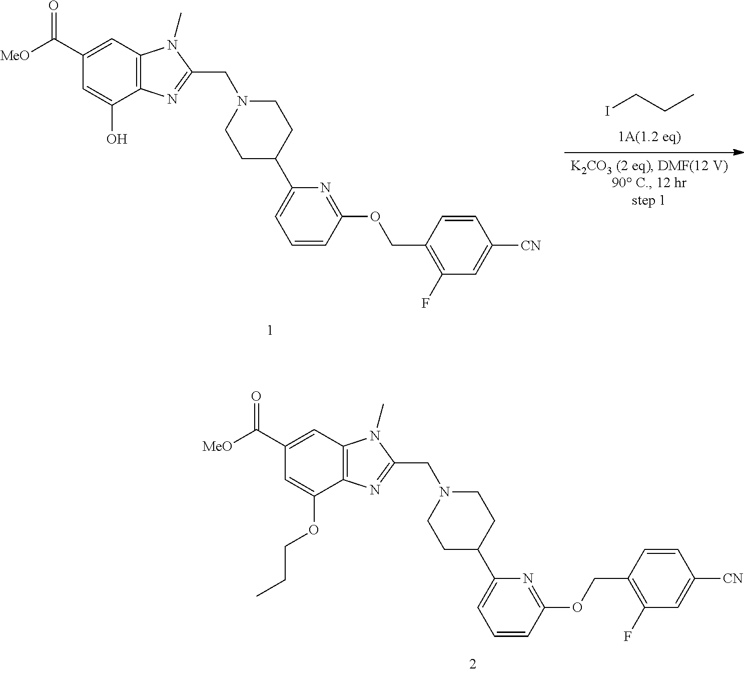

To a solution of 1 (130 mg, 245.49 μmol, 1 eq) in DMF (1.5 mL) was added K₂CO₃ (67.86 mg, 490.97 μmol, 2 eq) and 1A (50.08 mg, 294.58 μmol, 28.78 μL, 1.2 eq). The mixture was stirred at 90° C. for 12 hr. LC-MS showed 1 was consumed completely and desired mass was detected. The reaction mixture was diluted with H₂O (10 mL) and extracted with EtOAc (8 mL*3). The combined organic layers were washed with brine (8 mL*3), dried over anhydrous Na₂SO₄, filtered, and concentrated under reduced pressure to give a residue. The residue was purified by column chromatography (SiO₂, Petroleum ether/Ethyl acetate=1/1 to 0/1). 2 (140 mg, 244.91 μmol, 99.76% yield) was obtained as a white solid.

LCMS: RT=1.596 min, MS cal.: 571.2, [M+H]⁺=572.2

¹H NMR (400 MHz, CHCl3-d) δ=7.76 (s, 1H), 7.63 (br t, J=6.4 Hz, 1H), 7.53 (t, J=7.6 Hz, 1H), 7.44 (br d, J=7.6 Hz, 1H), 7.39 (br d, J=5.2 Hz, 2H), 6.76 (d, J=7.2 Hz, 1H), 6.65 (d, J=8.0 Hz, 1H), 5.50 (s, 2H), 5.31 (s, 1H), 4.22 (t, J=7.2 Hz, 2H), 3.90 (br s, 1H), 3.97 (s, 7H), 2.59 (br s, 1H), 2.28 (br s, 2H), 2.00 (sxt, J=7.2 Hz, 2H), 1.90-1.56 (m, 5H), 1.09 (t, J=7.6 Hz, 3H)

### General Procedure for Preparation of Compound 319 (3):

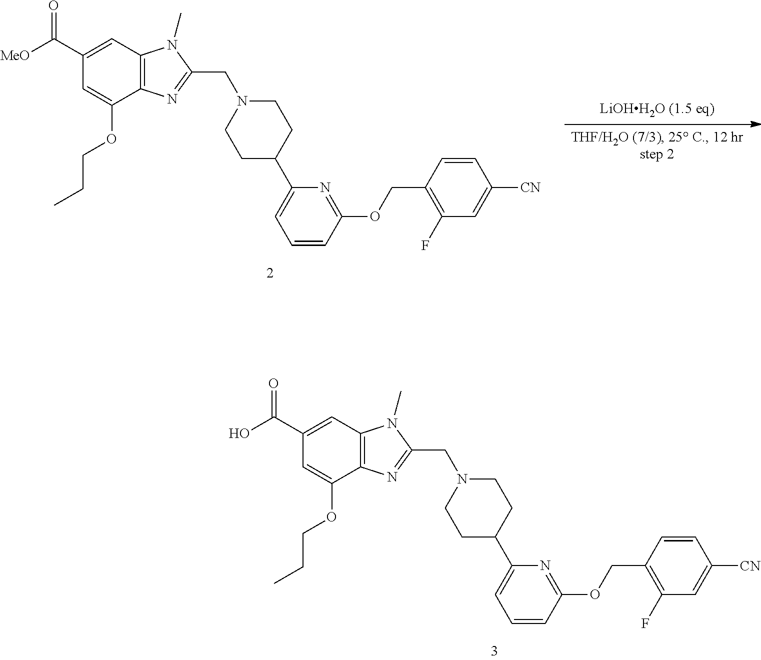 [SMILES: CCCOc1cc(C(=O)OC)cc2c1nc(CN1CCC(c3cccc(OCc4ccc(C#N)cc4F)n3)CC1)n2C ; "LiOH·H2O (1.5 eq)" ; "THF/H2O (7/3), 25° C., 12 hrstep 2" ; CCCOc1cc(C(=O)O)cc2c1nc(CN1CCC(c3cccc(OCc4ccc(C#N)cc4F)n3)CC1)n2C]

To a solution of 2 (130 mg, 227.42 μmol, 1 eq) in THF (1.4 mL) was added LiOH·H₂O (14.31 mg, 341.12 μmol, 1.5 eq) in H₂O (0.6 mL). The mixture was stirred at 25° C. for 12 hr. LC-MS (ET87210-51-P1A1) showed 2 was consumed completely and desired mass was detected. The residue was purified by prep-HPLC (column: Waters Xbridge Prep OBD C18 150*40 mm*10 um; mobile phase: [H₂O (10 mM NH₄HCO₃)-ACN]; gradient: 25%-60% B over 8.0 min). 2-((4-(6-((4-Cyano-2-fluorobenzyl)oxy)pyridin-2-yl)piperidin-1-yl)methyl)-1-methyl-4-propoxy-1H-benzo[d]imidazole-6-carboxylic acid (28.08 mg, 50.36 μmol, 22.14% yield, 99.79% purity) was obtained as a white solid.

LCMS: RT=2.384 min, MS cal.: 557.2, [M+H]⁺=558.2

HPLC: RT=10.950 min, purity: 99.79%

¹H NMR (400 MHz, DMSO-d₆) δ=7.88 (d, J=10.4 Hz, 1H), 7.77 (d, J=1.2 Hz, 1H), 7.73-7.67 (m, 2H), 7.67-7.60 (m, 1H), 7.25 (d, J=1.2 Hz, 1H), 6.87 (d, J=7.2 Hz, 1H), 6.71 (d, J=8.0 Hz, 1H), 5.45 (s, 2H), 4.16 (t, J=6.8 Hz, 2H), 3.90 (s, 3H), 3.80 (s, 2H), 2.90 (br d, J=11.2 Hz, 2H), 2.57 (br s, 1H), 2.18 (br t, J=10.4 Hz, 2H), 1.86-1.78 (m, 2H), 1.78-1.72 (m, 2H), 1.71-1.59 (m, 2H), 1.03 (t, J=7.2 Hz, 3H)

### Example A56—Preparation of Compound 320. 2-((4-(6-((4-Chloro-2-fluorobenzyl)oxy)pyridin-2-yl)piperidin-1-yl)methyl)-4-((1-hydroxypropan-2-yl)oxy)-1-methyl-1H-benzo[d]imidazole-6-carboxylic acid (Compound 320) (4)

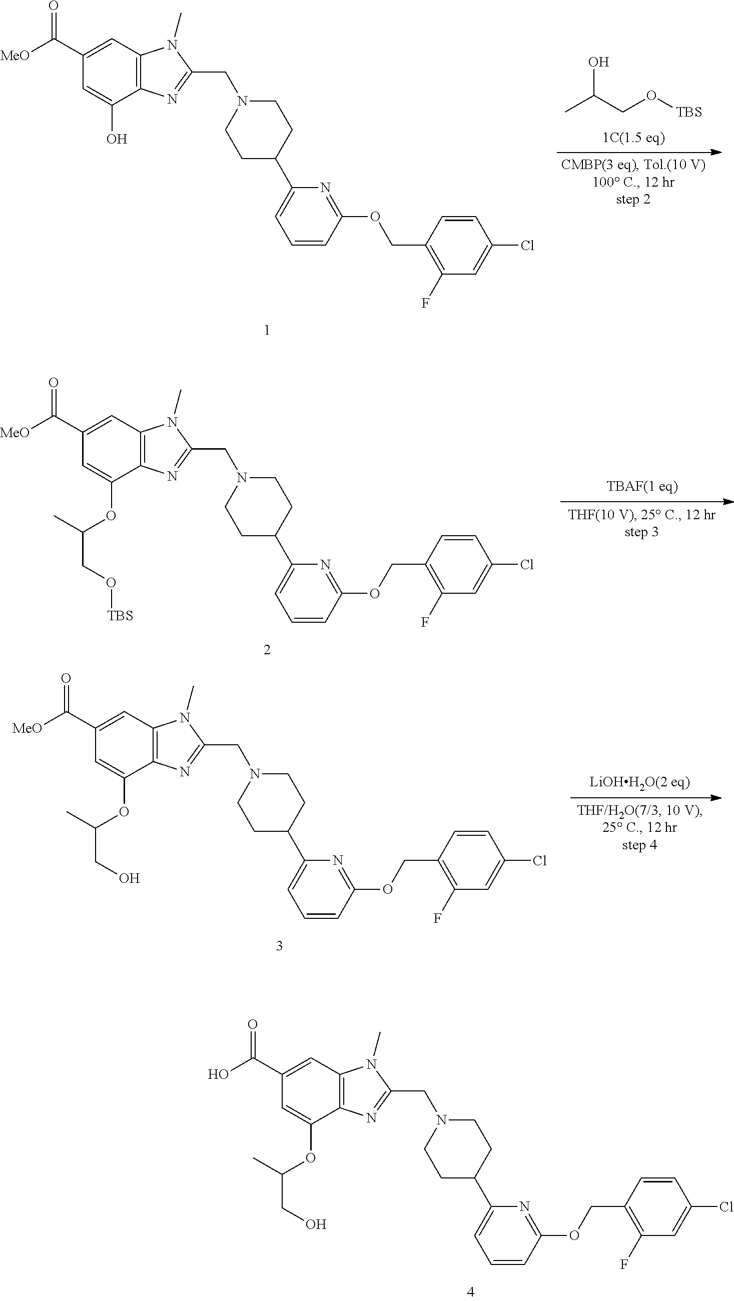

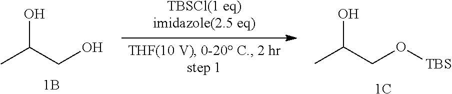

### General Procedure for Preparation of Intermediate 1C:

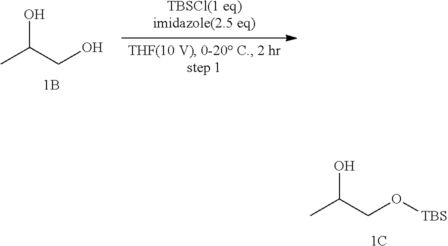

THF (40 mL) was charged to the 250 mL three-necked round bottom flask, then 1B (5 g, 65.71 mmol, 4.83 mL, 1 eq) and imidazole (6.71 g, 98.56 mmol, 1.5 eq) was added to the mixture at 25° C. At 0° C. (inner temperature), TBSCl (9.90 g, 65.71 mmol, 8.08 mL, 1 eq) in THF (10 mL) was added dropwise to the reaction mixture at 0° C. After the addition, the mixture was stirred at 25° C. for 12 hr. TLC (PE/EtOAc-3/1, R_{f}=0.4) indicated 1B was consumed completely and one new spot formed. The reaction was clean according to TLC. The suspension was filtered, and then the combined filtrates was diluted with NH₄Cl 50 mL and extracted with DCM 200 mL (50 mL*4). The combined organic layers were washed with NH₄Cl 50 mL (25 mL*2), dried over Na₂SO₄, filtered, and concentrated under reduced pressure to give a residue. 1C (4.8 g, 25.22 mmol, 38% yield) was obtained as a colorless oil.

¹HNMR (400 MHz, CHCl3-d) δ=3.86-3.77 (m, 1H), 3.59 (dd, J=3.2, 10.0 Hz, 1H), 3.35 (dd, J=8.0, 10.0 Hz, 1H), 2.52 (s, 1H), 1.12 (d, J=6.4 Hz, 3H), 0.91 (s, 9H), 0.09-0.05 (m, 6H)

### General Procedure for Preparation of Intermediate 2:

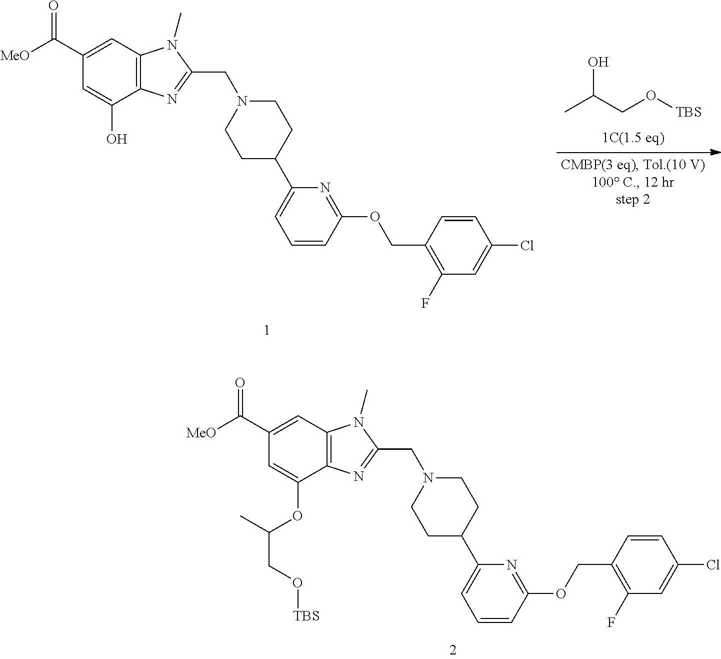

To a solution of 1 (200 mg, 371.06 μmol, 1 eq) (Synthesized from Int 3) and 1C (105.95 mg, 556.59 μmol, 1.5 eq) in Toluene (2 mL) was added CMBP (268.67 mg, 1.11 mmol, 3 eq). The mixture was stirred at 100° C. for 12 hr. LCMS (RT=2.068 min) showed 1 was consumed completely and one main peak with desired mass was detected. The reaction mixture was diluted with H₂O 10 mL and extracted with DCM (10 mL*3). The combined organic layers were dried over Na₂SO₄, filtered and concentrated under reduced pressure to give a residue. The residue was purified by column chromatography (SiO₂, Petroleum ether/Ethyl acetate=100/1 to 0/1). 2 (250 mg, 351.45 μmol, 94% yield) was obtained as a brown oil.

¹H NMR (400 MHz, CHCl3-d) δ=7.83-7.70 (m, 1H), 7.58-7.40 (m, 3H), 7.18-7.06 (m, 2H), 6.81-6.70 (m, 1H), 6.69-6.55 (m, 1H), 5.49-5.35 (m, 2H), 4.94-4.81 (m, 1H), 4.19-4.10 (m, 1H), 4.06-3.85 (m, 7H), 3.81-3.68 (m, 1H), 3.05-2.85 (m, 1H), 2.68-2.53 (m, 1H), 2.39-2.12 (m, 2H), 1.94-1.62 (m, 4H), 1.50-1.43 (m, 3H), 0.92-0.84 (m, 9H), 0.06 (d, J=14.4 Hz, 6H).

### General Procedure for Preparation of Intermediate 3:

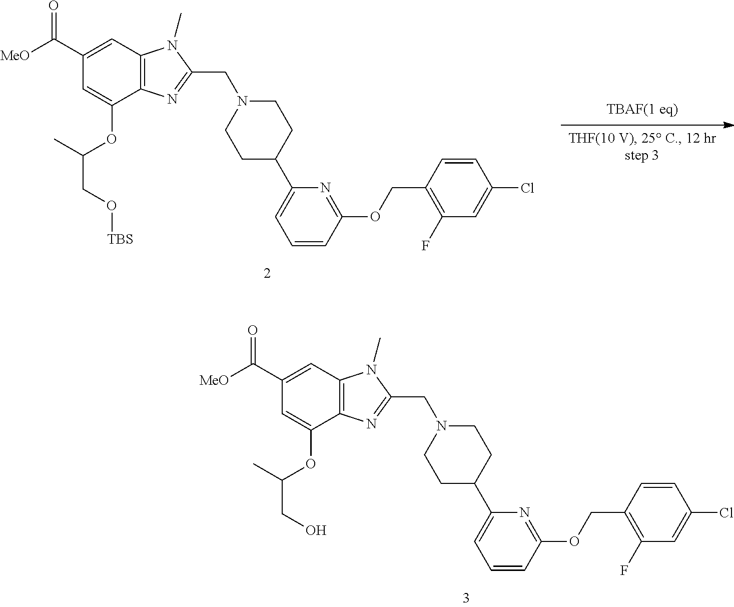

To a solution of 2 (200 mg, 281.16 μmol, 1 eq) in THF (2 mL) was added TBAF (1 M, 281.16 μL, 1 eq). The mixture was stirred at 25° C. for 12 hr. LCMS (RT=1.562 min) showed 2 was consumed completely and one main peak with desired mass was detected. The reaction mixture was diluted with H₂O 10 mL and extracted with DCM (10 mL*3). The combined organic layers were dried over Na₂SO₄, filtered and concentrated under reduced pressure to give a residue. 3 (170 mg, crude) was obtained as a yellow solid.

### General Procedure for Preparation of Compound 320 (4):

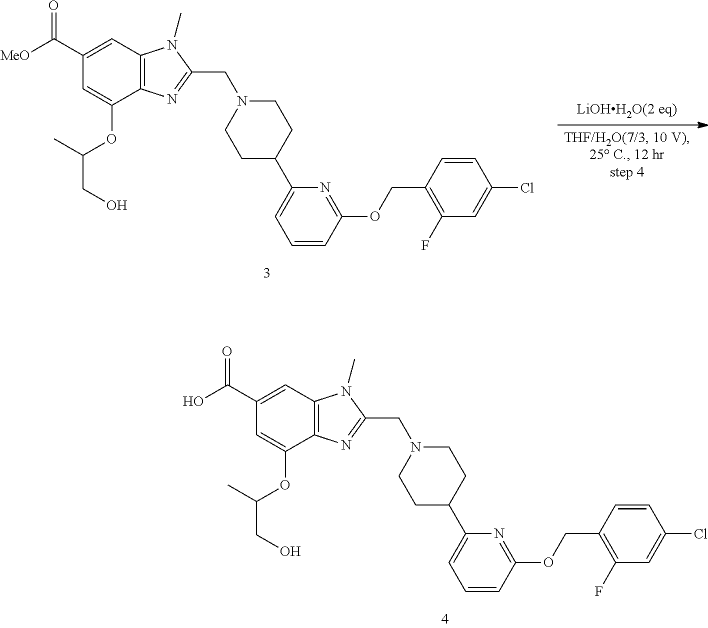 [SMILES: COC(=O)c1cc(OC(C)CO)c2nc(CN3CCC(c4cccc(OCc5ccc(Cl)cc5F)n4)CC3)n(C)c2c1 ; "LiOH·H2O(2 eq)" ; "THF/H2O(7/3, 10 V),25° C., 12 hrstep 4" ; CC(CO)Oc1cc(C(=O)O)cc2c1nc(CN1CCC(c3cccc(OCc4ccc(Cl)cc4F)n3)CC1)n2C]

A mixture of 3 (60 mg, 100.49 μmol, 1 eq), LiOH·H₂O (8.43 mg, 200.98 μmol, 2 eq) in THF (0.7 mL) and H₂O (0.3 mL) was degassed and purged with N₂ for 3 times, and then the mixture was stirred at 25° C. for 12 hr under N₂ atmosphere. LCMS (ET62365-805-p1b, RT=1.116 min) showed 3 was consumed completely and one main peak with desired mass was detected. The reaction mixture was filtered. The residue was purified by prep-HPLC (column: Waters Xbridge BEH C18 100*30 mm*10 um; mobile phase: [H₂O (10 mM NH₄HCO₃)-ACN]; gradient: 35%-65% B over 8.0 min). 2-((4-(6-((4-Chloro-2-fluorobenzyl)oxy)pyridin-2-yl)piperidin-1-yl)methyl)-4-((1-hydroxypropan-2-yl)oxy)-1-methyl-1H-benzo[d]imidazole-6-carboxylic acid (28.17 mg, 48.31 μmol, 28% yield) was obtained as a white solid.

LCMS: RT=2.318 min, MS cal.: 582.2, [M+H]⁺=583.2

HPLC: RT=10.862 min, purity: 100.00%

¹H NMR (400 MHz, MeOH-d4) δ=7.85 (d, J=1.0 Hz, 1H), 7.62-7.53 (m, 1H), 7.52-7.43 (m, 2H), 7.18 (s, 2H), 6.82 (d, J=7.2 Hz, 1H), 6.62 (d, J=8.1 Hz, 1H), 5.40 (s, 2H), 4.81-4.70 (m, 1H), 3.99 (s, 3H), 3.92 (s, 2H), 3.78 (dd, J=2.8, 4.4 Hz, 2H), 3.11-3.01 (m, 2H), 2.72-2.59 (m, 1H), 2.43-2.27 (m, 2H), 1.91-1.82 (m, 4H), 1.40 (d, J=6.3 Hz, 3H)

### Example A57—Preparation of Compound 321. 2-((4-(6-((4-Chloro-2-fluorophenyl)methoxy-d2)pyridin-2-yl)piperidin-1-yl)methyl-d2)-4-(difluoromethoxy)-1-methyl-1H-benzo[d]imidazole-6-carboxylic acid (Compound 321)

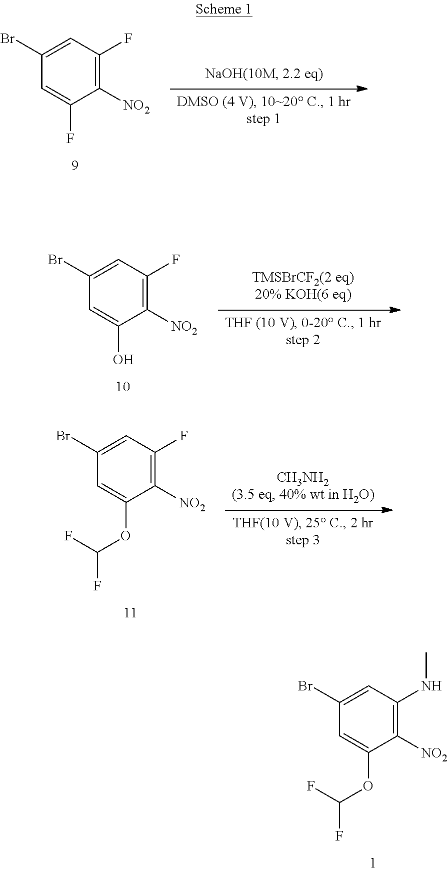

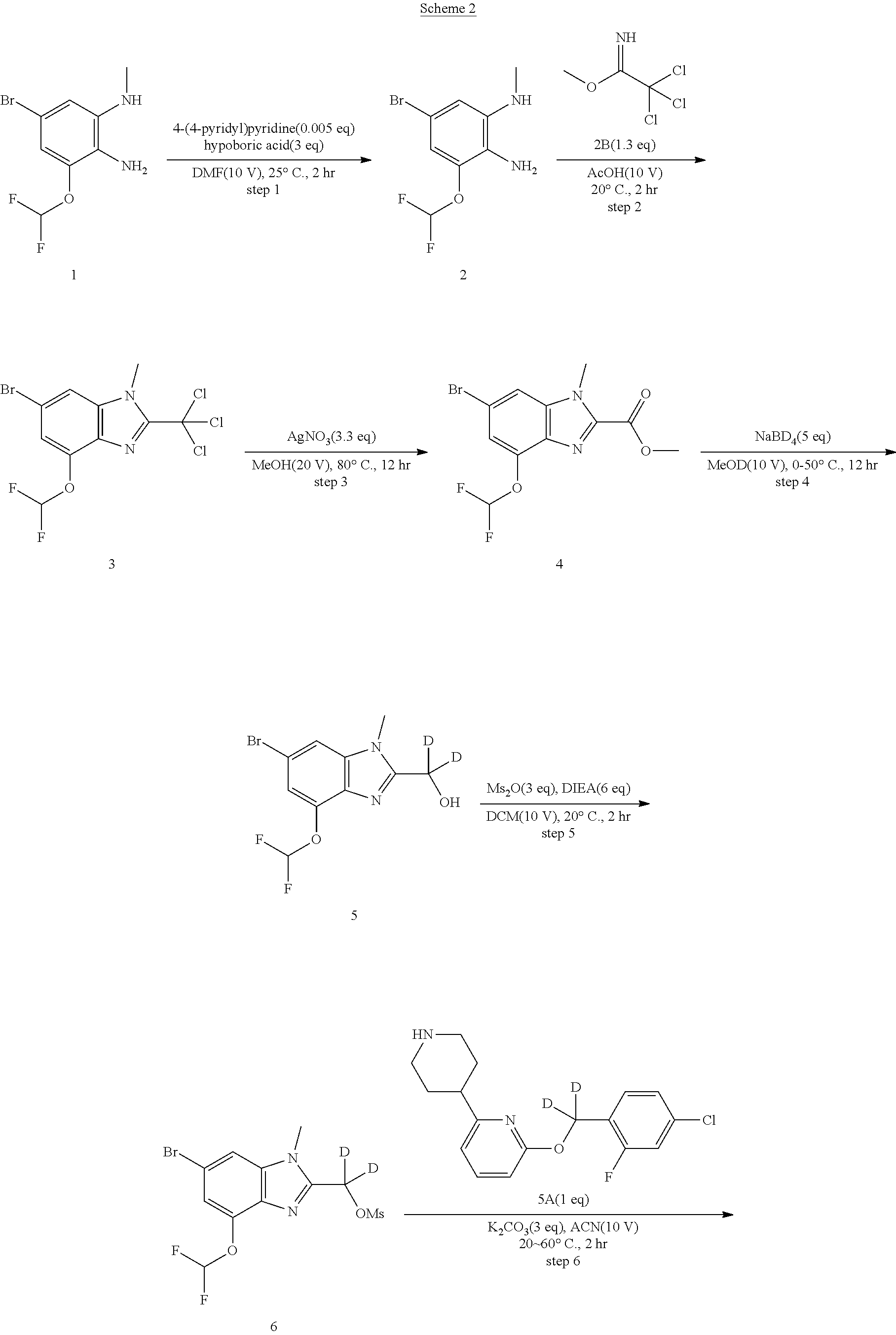

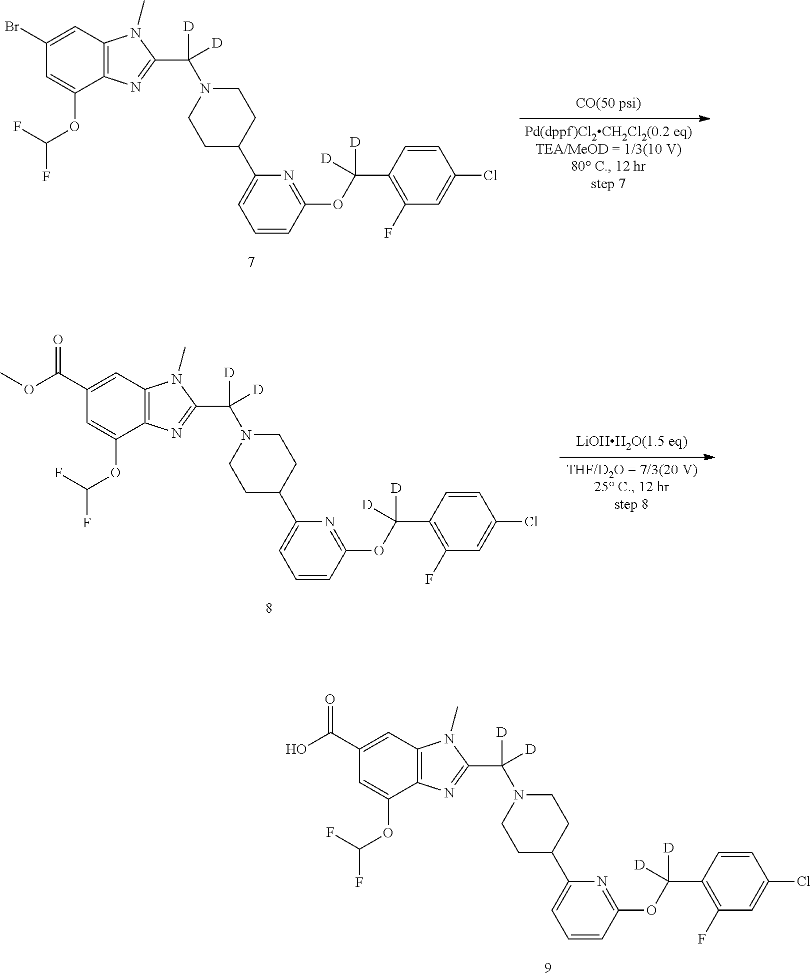

### General Procedure for Preparation of Scheme 1, Intermediate 10:

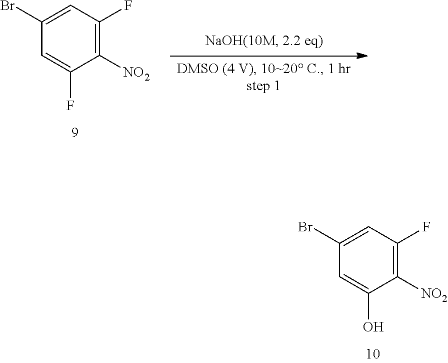

The reactions were carried out in parallels as two batches. To a mixture of 9 (100 g, 420.19 mmol, 1 eq) in DMSO (400 mL) was added NaOH (10 M, 92.44 mL, 2.2 eq) dropwise at 10-20° C. The mixture was stirred at 20° C. for 1 hr. The color of the mixture turned to brown. TLC (PE:EA=5:1, Rf=0.3) indicated 9 was consumed completely and one new spot formed. Two reactions were combined for work-up. The reaction mixture was acidified to pH=3 with 6M HCl (˜306 mL) at 0˜20° C. Then was diluted with H₂O (1.6 L) and extracted with MTBE (600 mL*2). The combined organic phases were washed with H₂O (600 mL*2) and brine (600 mL*2), dried with anhydrous Na₂SO₄, filtered and the filtrate was concentrated in vacuum to afford a yellow residue.

The crude product 10 (198 g, crude) as a yellow solid was used into the next step without further purification.

¹H NMR (400 MHz, DMSO-d6) δ=12.12 (br s, 1H), 7.32 (dd, J=2.0, 9.6 Hz, 1H), 7.10 (t, J=2.0 Hz, 1H)

### General Procedure for Preparation of Scheme 1, Intermediate 11:

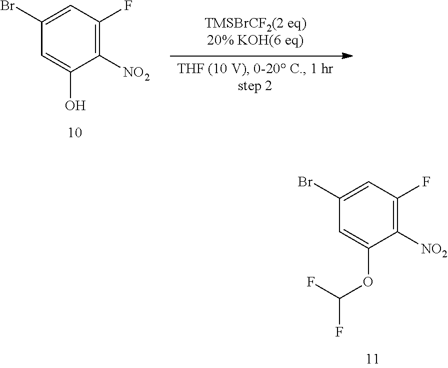 [SMILES: O=[N+]([O-])c1c(O)cc(Br)cc1F ; "THF (10 V), 0-20° C., 1 hrstep 2" ; "TMSBrCF2(2 eq)20% KOH(6 eq)" ; O=[N+]([O-])c1c(F)cc(Br)cc1OC(F)F]

THF (2 L) was charged to the three-necked round bottom flask, then 10 (198 g, 839.00 mmol, 1 eq) and KOH (1.88 kg, 6.71 mol, 20% purity, 6 eq) were added at 20° C. The mixture was cooled to 0° C. At 0° C., TMSBrCF₂ (340.80 g, 1.68 mol, 2 eq) in THF (200 mL) was added dropwise to the reaction mixture at 0° C. After the addition, the mixture was stirred at 20° C. for 1 hr. TLC (PE:EA=5:1, Rf=0.6) indicated 10 was consumed completely, the reaction was clean according to TLC. One major new spot with lower polarity was detected. The reaction mixture was diluted with H₂O (2 L) and extracted with MTBE (1 L*3). The combined organic layers were washed with brine (1 L), dried over Na₂SO₄, filtered, and concentrated under reduced pressure to give a residue. The crude product 11 (240 g, crude) as a brown oil was used into the next step without further purification.

¹H NMR (400 MHz, DMSO-d6) δ=7.98 (dd, J=1.6, 9.6 Hz, 1H), 7.76 (s, 1H), 7.49 (t, J=71.7 Hz, 1H)

### General Procedure for Preparation of Scheme 1, Intermediate 1:

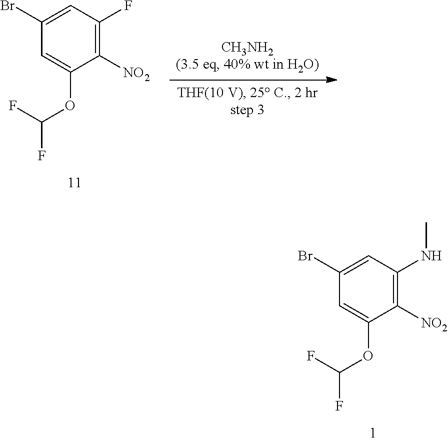 [SMILES: O=[N+]([O-])c1c(F)cc(Br)cc1OC(F)F ; "CH3NH2(3.5 eq, 40% wt in H2O)" ; "THF(10 V), 25° C., 2 hrstep 3" ; CNc1cc(Br)cc(OC(F)F)c1[N+](=O)[O-]]

THF (2.4 L) was charged to the 25 mL three-necked round bottom flask, then 11 (240 g, 839.15 mmol, 1 eq) was added to the mixture at 25° C. At 25° C., CH₃NH₂ (162.88 g, 2.10 mol, 2.5 eq, 40 wt % in H₂O) was added dropwise to the reaction mixture at 25° C. After the addition, the mixture was stirred at 25° C. for 1 hr. Another CH₃NH₂ (65.15 g, 839.15 mmol, 1 eq, 40 wt % in H₂O) was added dropwise to the reaction mixture at 25° C. After the addition, the mixture was stirred at 25° C. for 1 hr. TLC (PE:EA=5:1, Rf=0.4) indicated 11 was consumed completely and one new spot formed. The reaction was clean according to TLC. The reaction mixture was added FA (1 M) to pH=5 at 25° C. The reaction mixture was diluted with H₂O (2.4 L), extracted with MTBE (720 mL*3). The combined organic layer was washed with brine (720 mL), dried over Na₂SO₄, filtered and the filtrate was concentrated to give a crude product. The crude product was triturated with PE (1.2 L) at 25° C. for 30 min. The reaction mixture was filtered, and the filter cake was washed with PE (500 mL), then the filtrate was concentrated to give product. 1 (216 g, 706.56 mmol, 84% yield, 97.17% purity) was obtained as an orange solid.

LCMS: RT=1.314 min, MS cal.: 295.9, [M−H]⁺=294.9

¹H NMR (400 MHz, DMSO-d6) δ=7.27 (t, J=72.8 Hz, 1H)), 6.85 (d, J=1.7 Hz, 1H), 6.77 (q, J=4.2 Hz, 1H), 6.71 (d, J=0.7 Hz, 1H), 2.73 (d, J=4.6 Hz, 3H)

### General Procedure for Preparation of Scheme 2, Intermediate 2:

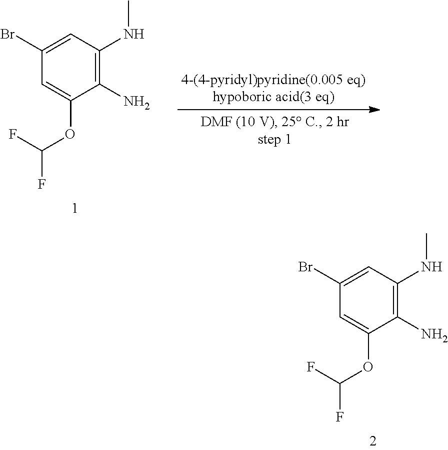

To a solution of 1 (2.5 g, 8.42 mmol, 1 eq) in DMF (50 mL) was added 4-(4-pyridyl)pyridine (6.57 mg, 42.08 μmol, 0.005 eq) and hypoboric acid (2.26 g, 25.25 mmol, 3 eq) at 25° C. The mixture was stirred at 25° C. for 2 hr. LC-MS showed 1 was consumed completely and desired m/z was detected. The reaction mixture was added H₂O (50 mL) at 0° C., and then extracted with Ethyl acetate (20 mL*3). The combined organic layers were washed with brine (30 mL*2), dried over Na₂SO₄, filtered, and concentrated under reduced pressure to give a crude product. The residue was purified by column chromatography (SiO₂, Petroleum ether/Ethyl acetate=1/0 to 20/1). 2 (3.7 g, 13.85 mmol, 82% yield) was obtained as a brown oil.

LCMS: RT=1.197 min, MS cal.: 265.9, 267.9, [M+H]⁺=266.8

1H NMR (400 MHz, CHCl3-d) δ=6.72 (s, 1H), 6.64 (d, J=2.0 Hz, 1H), 6.46 (t, J=74.0 Hz, 1H), 2.87 (s, 3H)

### General Procedure for Preparation of Scheme 2, Intermediate 3:

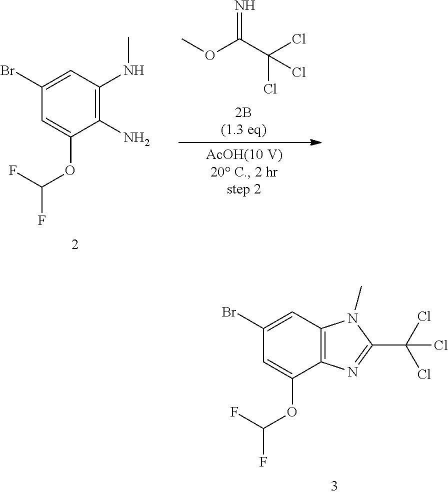

To a solution of 2 (500 mg, 1.87 mmol, 1 eq) in AcOH (5 mL) was added 2B (429.40 mg, 2.43 mmol, 301.33 μL, 1.3 eq) dropwise slowly at 0° C. The mixture was stirred at 20° C. for 2 hr. LC-MS showed 2 was consumed completely and desired m/z was detected. The reaction mixture was added to H₂O (20 mL) at 0° C., and then extracted with Ethyl acetate (10 mL*3). The combined organic layers were washed with brine (10 mL*2), dried over Na₂SO₄, filtered, and concentrated under reduced pressure to give a crude product. The residue was purified by column chromatography (SiO₂, Petroleum ether/Ethyl acetate=99/1 to 5/1). 3 (670 mg, 1.70 mmol, 90% yield) was obtained as a yellow solid.

LCMS: RT=0.608 min, MS cal.: 391.9, 393.9, [M+H]⁺=394.9

1H NMR (400 MHz, CHCl3-d) δ=7.53 (t, J=74.0 Hz, 1H), 7.42 (d, J=1.6 Hz, 1H), 7.26 (d, J=1.2 Hz, 1H), 4.09 (s, 3H)

### General Procedure for Preparation of Scheme 2, Compound 4:

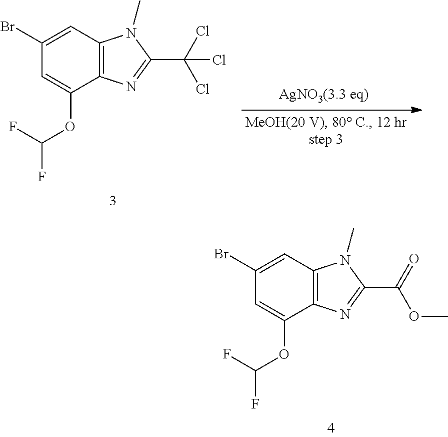 [SMILES: Cn1c(C(Cl)(Cl)Cl)nc2c(OC(F)F)cc(Br)cc21 ; COC(=O)c1nc2c(OC(F)F)cc(Br)cc2n1C]

MeOH (12 mL) was charged to the three-necked round bottom flask, 3 (1.2 g, 3.04 mmol, 1 eq) was added to the mixture at 25° C. At 25° C., AgNO₃ (1.71 g, 10.04 mmol, 3.3 eq) was added in portions to the reaction mixture at 25° C. After the addition, the mixture was stirred at 80° C. for 12 hr. LCMS showed 3 was consumed completely and desired m/z was detected. The reaction mixture was adjusted to pH=7˜8 with aq. NaHCO₃ at 25° C. The mixture was extracted by MTBE (15 mL*3). The combined organic layers were washed with brine (10 mL), dried over Na₂SO₄ filtered and concentrated under reduced pressure to give a crude product. The reaction mixture was concentrated to give 4 (530 mg, crude) was obtained as a yellow solid.

LCMS: RT=1.206 min, MS cal.: 335.10, [M+H]⁺=334.9

### General Procedure for Preparation of Scheme 2, Intermediate 5:

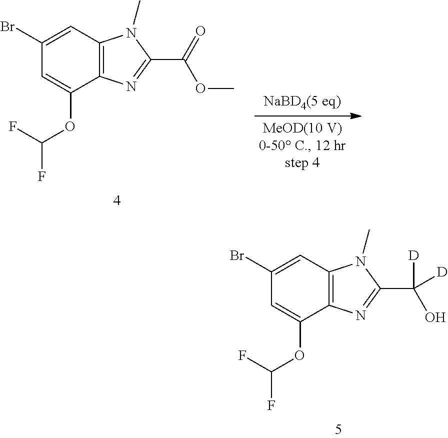 [SMILES: COC(=O)c1nc2c(OC(F)F)cc(Br)cc2n1C ; [2H]C([2H])(O)c1nc2c(OC(F)F)cc(Br)cc2n1C]

To a solution of 4 (330 mg, 984.78 μmol, 1 eq) in MeOD (3.3 mL) was added NaBD4 (186.28 mg, 4.92 mmol, 5 eq) at 0° C. The mixture was stirred at 50° C. for 12 hr. LC-MS showed 4 was consumed completely and one main peak with desired m/z was detected. The reaction mixture was quenched by addition D20 5 mL at 20° C., and then diluted with H₂O 5 mL and extracted with EtOAc 10 mL. The combined organic layers were washed with brine 5 ml, dried over Na₂SO₄, filtered, and concentrated under reduced pressure to give a crude product. The residue was purified by prep-HPLC (column: Waters Xbridge BEH C18 250*50 mm*10 um; mobile phase: [H₂O (10 mM NH₄HCO₃)-ACN]; gradient: 20%-60% B over 10.0 min). 5 (70 mg, 226.46 μmol, 23% yield) was obtained as a white solid.

LCMS: RT=1.057 min, MS cal.: 308.0, 310.0, [M+H]⁺=308.9

¹H NMR (400 MHz, DMSO-d₆) δ=7.77 (d, J=1.60 Hz, 1H), 7.69 (t, J=74.4 Hz, 1H), 7.16 (d, J=1.60 Hz, 1H), 3.84 (s, 3H)

### General Procedure for Preparation of Scheme 2, Intermediate 6:

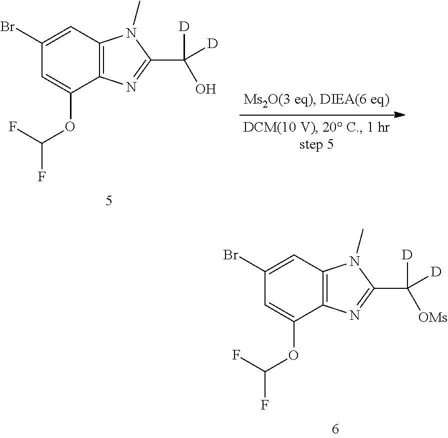 [SMILES: [2H]C([2H])(O)c1nc2c(OC(F)F)cc(Br)cc2n1C ; [2H]C([2H])(OS(C)(=O)=O)c1nc2c(OC(F)F)cc(Br)cc2n1C]

To a solution of 5 (50 mg, 161.76 μmol, 1 eq) and DIEA (125.44 mg, 970.55 μmol, 169.05 μL, 6 eq) in DCM (1.5 mL) was added methylsulfonyl methanesulfonate (84.53 mg, 485.27 μmol, 3 eq). The mixture was stirred at 20° C. for 1 hr. LC-MS showed 5 was consumed completely and one main peak with desired m/z was detected. The mixture was added HCOOH (1M, 3 mL) to adjust pH=6, diluted with H₂O (5 mL), extracted with Ethyl acetate (5 mL*3). The combined organic layer was washed with 10 mL brine, dried over Na₂SO₄, filtered and the filtrate was concentrated to give a residue. 6 (40 mg, 103.31 μmol, 64% yield) was obtained as a yellow oil.

LCMS: RT=0.474 min, MS cal.: 386.0, 388.0, [M+H]⁺=386.9

### General Procedure for Preparation of Scheme 2, Intermediate 7:

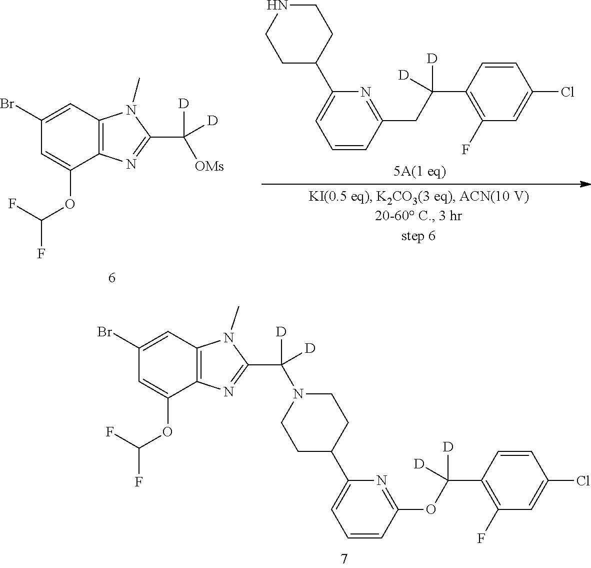 [SMILES: [2H]C([2H])(OS(C)(=O)=O)c1nc2c(OC(F)F)cc(Br)cc2n1C ; [2H]C([2H])(Cc1cccc(C2CCNCC2)n1)c1ccc(Cl)cc1F ; [2H]C([2H])(Oc1cccc(C2CCN(C([2H])([2H])c3nc4c(OC(F)F)cc(Br)cc4n3C)CC2)n1)c1ccc(Cl)cc1F]

To a solution of 6 (100 mg, 258.27 μmol, 1 eq), 5A (83.37 mg, 258.27 μmol, 1 eq) (Synthesized from Int 4) in ACN (1 mL) was added KI (21.44 mg, 129.13 μmol, 0.5 eq) and K₂CO₃ (107.08 mg, 774.81 μmol, 3 eq). The mixture was stirred at 60° C. for 3 hr. LC-MS showed 6 was consumed completely and one main peak with desired m/z was detected. The mixture was diluted with H₂O (5 mL), extracted with Ethyl acetate (5 mL*3). The combined organic layer was washed with 10 mL brine, dried over Na₂SO₄, filtered and the filtrate was concentrated to give a crude product. The residue was purified by column chromatography (SiO₂, Petroleum ether/Ethyl acetate=1/1). 7 (100 mg, 162.90 μmol, 63% yield) was obtained as a yellow oil.

LCMS: RT=1.866 min, MS cal.: 612.1, 614.1, [M+H]⁺=613.0

### General Procedure for Preparation of Scheme 2, Intermediate 8:

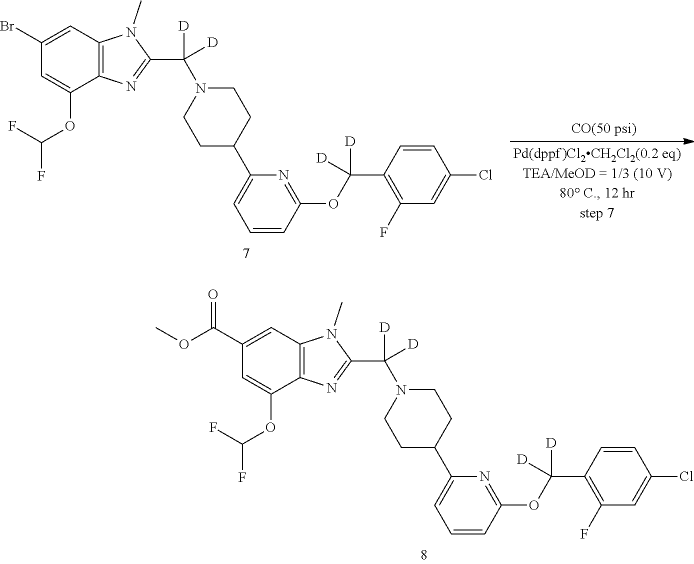 [SMILES: [2H]C([2H])(Oc1cccc(C2CCN(C([2H])([2H])c3nc4c(OC(F)F)cc(Br)cc4n3C)CC2)n1)c1ccc(Cl)cc1F ; [2H]C([2H])(Oc1cccc(C2CCN(C([2H])([2H])c3nc4c(OC(F)F)cc(C(=O)OC)cc4n3C)CC2)n1)c1ccc(Cl)cc1F]

MeOD (3 mL) and TEA (1 mL) was charged to the 35 mL Hydrogenated bottle, then 7 (50 mg, 81.45 μmol, 1 eq) and Pd(dppf)Cl₂·CH₂Cl₂ (13.30 mg, 16.29 μmol, 0.2 eq) was added at 20° C. After the addition, the mixture was degassed and purged with CO for 3 times. The mixture was stirred at 80° C. (50 psi) for 12 hr. The reaction was monitored by LCMS showed 7 was consumed completely and one main peak with desired m/z was detected. TLC (Petroleum ether:Ethyl acetate=0:1) indicated 7 was consumed completely and one new spot formed. The reaction was clean according to TLC. After 12 hr, the reaction mixture was diluted with H₂O (3 mL), extracted by Ethyl acetate 10 mL (5 mL*2). Then organic phase was combined and washed by brine 5 mL, dried with Na₂SO₄, filtered. The organic phase was concentrated under reduced pressure at 40° C. to give a residue. The residue was purified by column chromatography (SiO₂, Petroleum ether/Ethyl acetate=100/0 to 1/1). 8 (20 mg, 29.00 μmol, 35.61% yield, 86% purity) was obtained as a green oil.

LCMS: Rt=1.753 min, MS cal.: 592.20, 594.20, [M+H]⁺=593.1

¹H NMR (400 MHz, CHCl3-d) δ=8.12 (d, J=1.2 Hz, 1H), 7.65 (t, J=78.8 Hz, 1H), 7.62 (t, J=7.8 Hz, 1H), 7.58-7.52 (m, 2H), 7.47-7.42 (m, 1H), 7.28 (dd, J=2.0, 8.4 Hz, 1H), 6.86 (d, J=7.2 Hz, 1H), 6.66 (d, J=8.0 Hz, 1H), 3.98 (s, 3H), 3.90 (s, 3H), 2.94 (br d, J=11.6 Hz, 2H), 2.64-2.54 (m, 1H), 2.27-2.18 (m, 2H), 1.82-1.66 (m, 5H)

### General Procedure for Preparation of Scheme 2, Compound 321 (9):

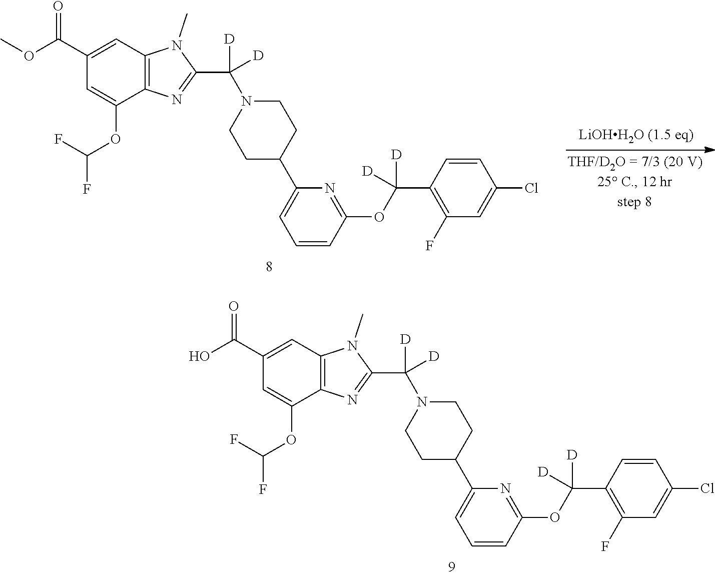 [SMILES: [2H]C([2H])(Oc1cccc(C2CCN(C([2H])([2H])c3nc4c(OC(F)F)cc(C(=O)OC)cc4n3C)CC2)n1)c1ccc(Cl)cc1F ; [2H]C([2H])(Oc1cccc(C2CCN(C([2H])([2H])c3nc4c(OC(F)F)cc(C(=O)O)cc4n3C)CC2)n1)c1ccc(Cl)cc1F]

To a solution of 8 (25 mg, 41.94 μmol, 1 eq) in THF (0.35 mL) was added LiOH·H₂O (2.64 mg, 62.91 μmol, 1.5 eq) and H₂O (0.15 mL). The mixture was stirred at 25° C. for 12 hr. LCMS showed 8 was consumed completely and one main peak with desired m/z was detected. The residue was purified by prep-HPLC (column: Waters Xbridge BEH C18 100*30 mm*10 um; mobile phase: [H₂O (10 mM NH₄HCO₃)-ACN]; gradient: 20%-65% B over 8.0 min). 2-((4-(6-((4-Chloro-2-fluorophenyl)methoxy-d2)pyridin-2-yl)piperidin-1-yl)methyl-d2)-4-(difluoromethoxy)-1-methyl-1H-benzo[d]imidazole-6-carboxylic acid (11.16 mg, 18.70 μmol, 44.58% yield, 97% purity) was obtained as a white solid.

LCMS: Rt=2.806 min, MS cal.: 578.18, 580.18, [M+H]⁺=579.2

HPLC: Rt=12.233 min, purity=97.40%

¹H NMR (400 MHz, DMSO-d₄) δ=8.05 (s, 1H), 7.62 (t, J=10 Hz, 1H), 7.62 (t, J=74.6 Hz, 1H) 7.58-7.53 (m, 1H), 7.51 (s, 1H), 7.47-7.43 (m, 1H), 7.28 (dd, J=2.0, 8.4 Hz, 1H), 6.86 (d, J=7.6 Hz, 1H), 6.66 (d, J=8.4 Hz, 1H), 3.96 (s, 3H), 2.97-2.90 (m, 2H), 2.64 (s, 1H), 2.27-2.16 (m, 2H), 1.82-1.63 (m, 4H)

### Example A58—Preparation of Compound 322. 2-((4-(6-((4-chloro-2-fluorobenzyl)oxy)pyridin-2-yl)piperidin-1-yl)methyl)-1-methyl-4-propoxy-1H-benzo[d]imidazole-6-carboxylic acid (Compound 322) (3)

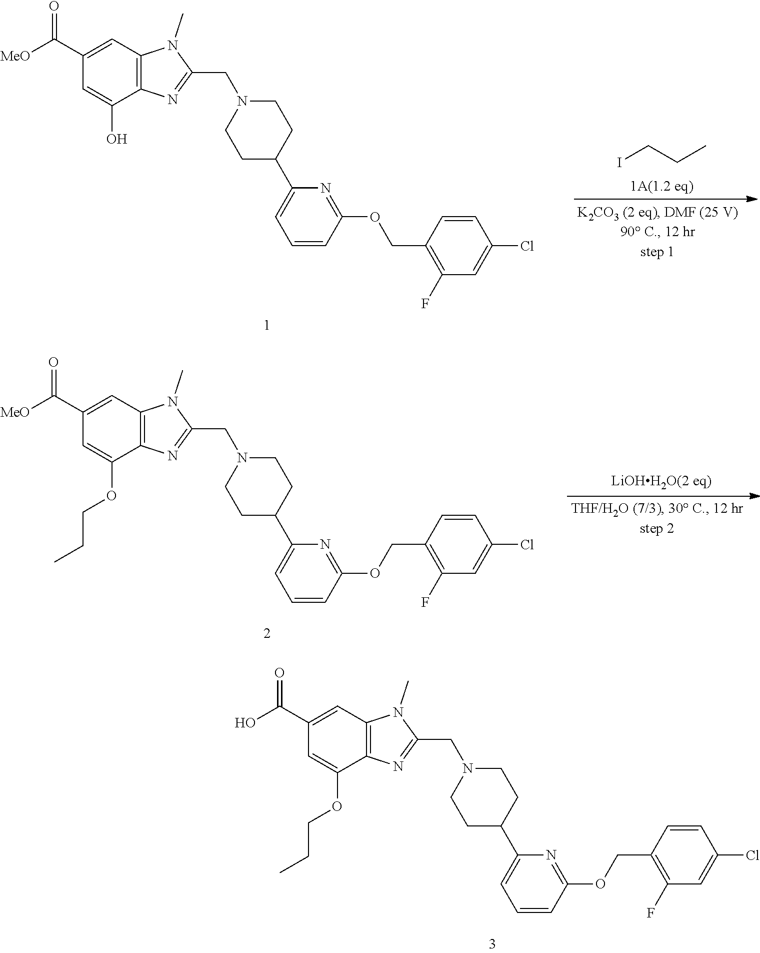

### General Procedure for Preparation of Intermediate 2:

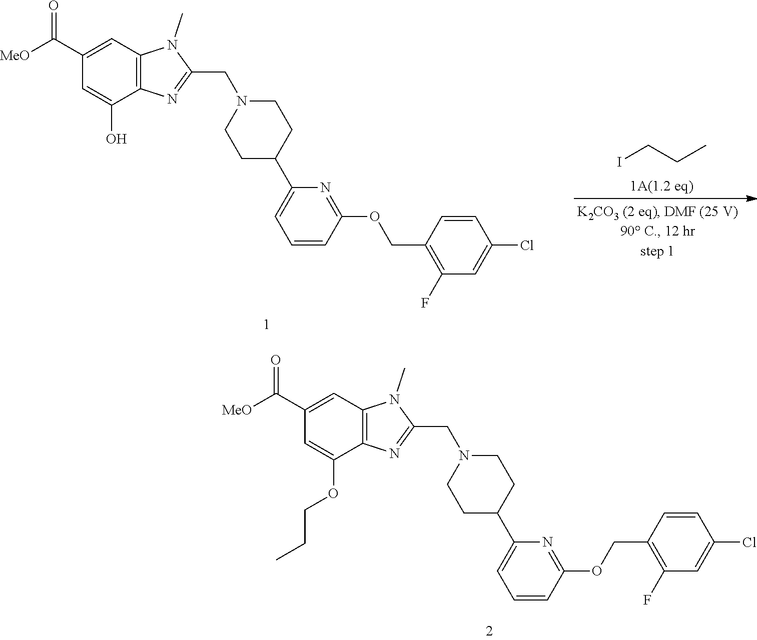

To a solution of 1 (80 mg, 148.42 μmol, 1 eq) in DMF (2 mL) was added K₂CO₃ (41.03 mg, 296.85 μmol, 2 eq) and 1A (30.28 mg, 178.11 μmol, 17.40 μL, 1.2 eq) at 20° C. The mixture was stirred at 90° C. for 12 hr. LC-MS showed 1 was consumed completely and desired mass was detected. The reaction mixture was diluted with H₂O (10 mL) and extracted with EtOAc (8 mL*3). The combined organic layers were washed with brine (8 mL*3), dried over anhydrous Na₂SO₄, filtered and concentrated under reduced pressure to give a residue. The residue was purified by column chromatography (SiO₂, Petroleum ether/Ethyl acetate=3/1 to 0/1). 2 (55 mg, 94.65 μmol, 63.77% yield) was obtained as a white solid.

LCMS: RT=1.778 min, MS cal.: 580.2, [M+H]⁺=581.1

¹H NMR (400 MHz, CHCl3-d) δ=7.76 (s, 1H), 7.56-7.35 (m, 3H), 7.17-7.08 (m, 2H), 6.73 (d, J=7.2 Hz, 1H), 6.60 (d, J=8.0 Hz, 1H), 5.41 (s, 2H), 4.22 (t, J=6.8 Hz, 2H), 4.02-3.94 (m, 6H), 3.90 (s, 2H), 2.96 (br d, J=11.2 Hz, 2H), 2.61 (br t, J=11.6 Hz, 1H), 2.29 (br t, J=10.4 Hz, 2H), 2.03-1.95 (m, 2H), 1.93-1.76 (m, 4H), 1.09 (t, J=7.2 Hz, 3H)

### General Procedure for Preparation of Compound 322 (3):

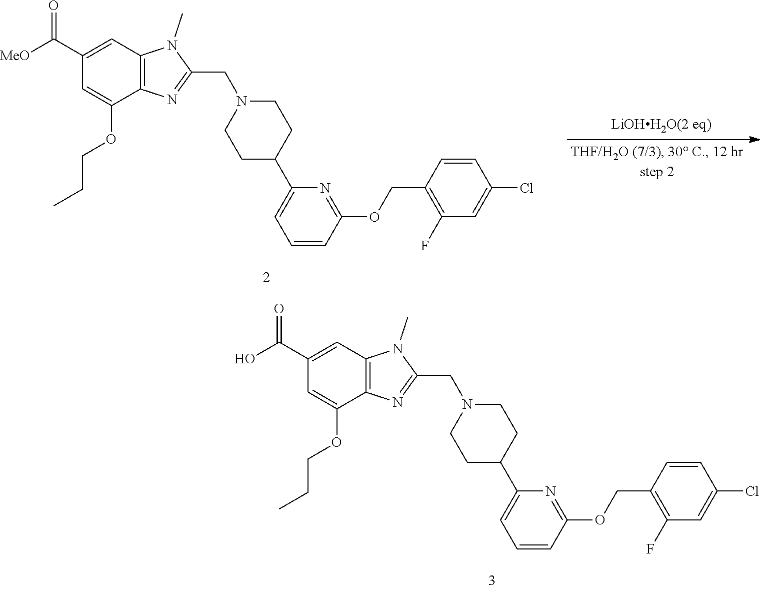 [SMILES: CCCOc1cc(C(=O)OC)cc2c1nc(CN1CCC(c3cccc(OCc4ccc(Cl)cc4F)n3)CC1)n2C ; CCCOc1cc(C(=O)O)cc2c1nc(CN1CCC(c3cccc(OCc4ccc(Cl)cc4F)n3)CC1)n2C]

To a solution of 2 (45 mg, 77.44 μmol, 1 eq) in THF (0.35 mL) was added LiOH·H₂O (4.87 mg, 116.16 μmol, 1.5 eq) in H₂O (0.15 mL). The mixture was stirred at 30° C. for 4 hr. LC-MS showed ˜62% of 2 remained. Several new peaks were shown on LC-MS and ˜33% of desired Compound was detected. So LiOH·H₂O (1.62 mg, 38.72 μmol, 0.5 eq) was added the mixture, the mixture was stirred at 30° C. for 8 hr. LC-MS showed 2 was consumed completely and desired mass was detected. The residue was purified by prep-HPLC (column: Waters Xbridge Prep OBD C18 150*40 mm*10 um; mobile phase: [H₂O (10 mM NH₄HCO₃)-ACN]; gradient: 20%-70% B over 8.0 min). 2-((4-(6-((4-chloro-2-fluorobenzyl)oxy)pyridin-2-yl)piperidin-1-yl)methyl)-1-methyl-4-propoxy-1H-benzo[d]imidazole-6-carboxylic acid (28.85 mg, 50.88 μmol, 65.70% yield, 96.96% purity) was obtained as a white solid.

LCMS: RT=2.512 min, MS cal.: 558.2, [M+H]⁺=567.2

HPLC: RT=12.514 min, purity: 96.96%

¹H NMR (400 MHz, DMSO-d₆) δ=7.76 (d, J=1.2 Hz, 1H), 7.62 (dd, J=7.6, 8.2 Hz, 1H), 7.55 (t, J=8.0 Hz, 1H), 7.45 (dd, J=2.0, 9.6 Hz, 1H), 7.28 (dd, J=1.6, 8.3 Hz, 1H), 7.26 (d, J=1.2 Hz, 1H), 6.86 (d, J=7.2 Hz, 1H), 6.66 (d, J=8.0 Hz, 1H), 5.36 (s, 2H), 4.16 (t, J=6.8 Hz, 2H), 3.90 (s, 3H), 3.81 (s, 2H), 2.92 (br d, J=11.2 Hz, 2H), 2.63-2.54 (m, 1H), 2.26-2.12 (m, 2H), 1.91-1.58 (m, 6H), 1.03 (t, J=7.6 Hz, 3H)

### Example A59—Preparation of Compound 323. 2-((4-(6-((4-Cyano-2-fluorobenzyl)oxy)pyridin-2-yl)piperidin-1-yl)methyl)-7-methoxy-3-methyl-3H-imidazo[4,5-b] pyridine-5-carboxylic acid (Compound 323) (3)

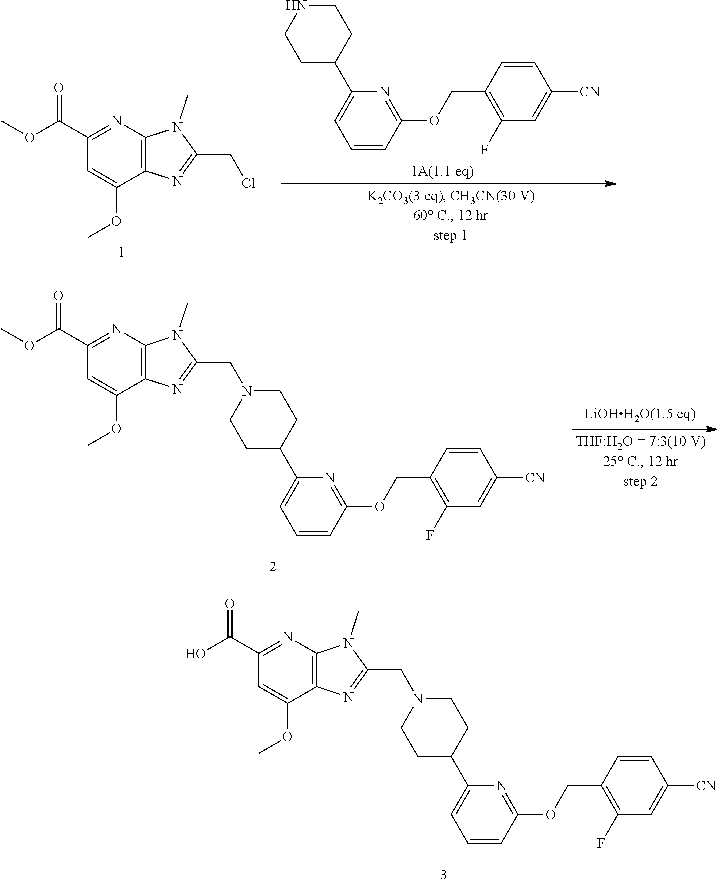 [SMILES: COC(=O)c1cc(OC)c2nc(CCl)n(C)c2n1 ; N#Cc1ccc(COc2cccc(C3CCNCC3)n2)c(F)c1 ; COC(=O)c1cc(OC)c2nc(CN3CCC(c4cccc(OCc5ccc(C#N)cc5F)n4)CC3)n(C)c2n1 ; COc1cc(C(=O)O)nc2c1nc(CN1CCC(c3cccc(OCc4ccc(C#N)cc4F)n3)CC1)n2C]

### General Procedure for Preparation of Intermediate 2:

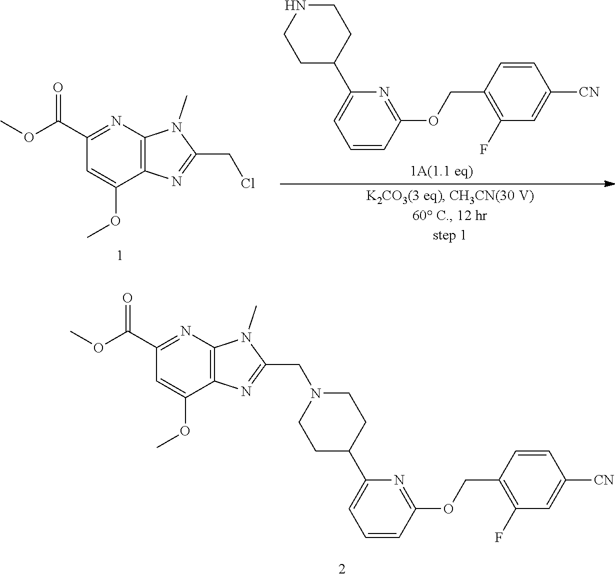 [SMILES: COC(=O)c1cc(OC)c2nc(CCl)n(C)c2n1 ; N#Cc1ccc(COc2cccc(C3CCNCC3)n2)c(F)c1 ; COC(=O)c1cc(OC)c2nc(CN3CCC(c4cccc(OCc5ccc(C#N)cc5F)n4)CC3)n(C)c2n1]

To a solution of 1 (70 mg, 259.56 μmol, 1 eq) in ACN (2 mL) was added 1A (88.90 mg, 285.52 μmol, 1.1 eq, synthesized from Int 3) and K₂CO₃ (107.62 mg, 778.69 μmol, 3 eq) at 15° C., the mixture was stirred at 60° C. for 12 hr. LCMS showed 1 was consumed completely and one main peak with desired m/z was detected. The reaction mixture was diluted with H₂O (5 mL) and extracted with EtOAc (5 mL*3). The combined organic layers were washed with brine (5 mL*2), dried over Na₂SO₄, filtered and concentrated under reduced pressure to give a residue. The residue was purified by column chromatography (SiO₂, Petroleum ether/Ethyl acetate=1/0 to 1/1) to obtained 2 (72 mg, 132.21 μmol, 50.94% yield) as a white solid.

LCMS: RT=1.885 min, MS cal.: 544.59, [M+H]⁺=545.1

¹H NMR (400 MHz, CHCl3-d) δ=7.67-7.59 (m, 2H), 7.53 (t, J=7.7 Hz, 1H), 7.44 (br d, J=8.0 Hz, 1H), 7.37 (dd, J=1.3, 9.2 Hz, 1H), 6.75 (d, J=7.4 Hz, 1H), 6.64 (d, J=8.2 Hz, 1H), 5.50 (s, 2H), 4.15 (s, 3H), 4.06 (s, 3H), 4.04 (s, 3H), 3.95-3.82 (m, 2H), 2.95 (br d, J=9.4 Hz, 2H), 2.58 (br s, 1H), 2.28 (br t, J=10.3 Hz, 2H), 1.94-1.70 (m, 4H)

### General Procedure for Preparation of Compound 323 (3):

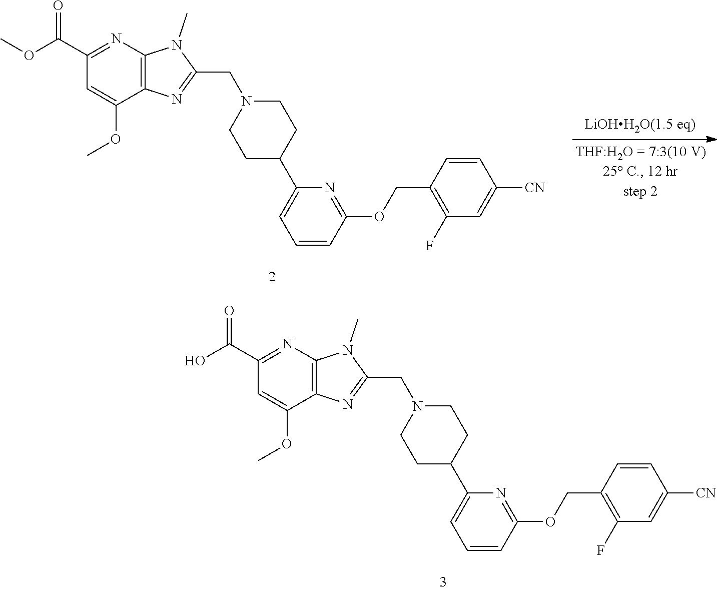 [SMILES: COC(=O)c1cc(OC)c2nc(CN3CCC(c4cccc(OCc5ccc(C#N)cc5F)n4)CC3)n(C)c2n1 ; COc1cc(C(=O)O)nc2c1nc(CN1CCC(c3cccc(OCc4ccc(C#N)cc4F)n3)CC1)n2C]

To a solution of 2 (62 mg, 113.85 μmol, 1 eq) in THF (0.7 mL) and H₂O (0.3 mL) was added LiOH·H₂O (7.17 mg, 170.78 μmol, 1.5 eq) at 15° C., the mixture was stirred at 25° C. for 12 hr. LCMS showed 2 was consumed completely and one main peak with desired m/z was detected. The reaction mixture was filtered and purified by prep-HPLC (neutral condition; column: Waters Xbridge Prep OBD C18 150*40 mm*10 um; mobile phase: [H₂O (10 mM NH₄HCO₃)-ACN]; gradient: 15%-65% B over 8.0 min) to obtained 2-((4-(6-((4-Cyano-2-fluorobenzyl)oxy)pyridin-2-yl)piperidin-1-yl)methyl)-7-methoxy-3-methyl-3H-imidazo[4,5-b] pyridine-5-carboxylic acid (29.02 mg, 54.70 μmol, 48.04% yield) as a white solid.

LCMS: RT=1.057 min, MS cal.: 530.56, [M+H]⁺=531.1

¹H NMR (400 MHz, MeOH-d4) δ=7.68 (s, 1H), 7.70-7.64 (m, 1H), 7.62-7.55 (m, 2H), 7.53 (dd, J=1.4, 7.9 Hz, 1H), 6.84 (d, J=7.3 Hz, 1H), 6.68 (d, J=8.0 Hz, 1H), 5.51 (s, 2H), 4.14 (s, 3H), 4.03 (s, 3H), 3.99 (s, 2H), 3.15-3.07 (m, 2H), 2.72-2.61 (m, 1H), 2.49-2.38 (m, 2H), 1.92-1.78 (m, 4H)

### Example A60—Preparation of Compound 324. 2-((4-(6-((4-Chloro-2-fluorobenzyl)oxy)pyridin-2-yl)piperidin-1-yl)methyl)-7-methoxy-3-methyl-3H-imidazo[4,5-b]pyridine-5-carboxylic acid (Compound 324) (6)

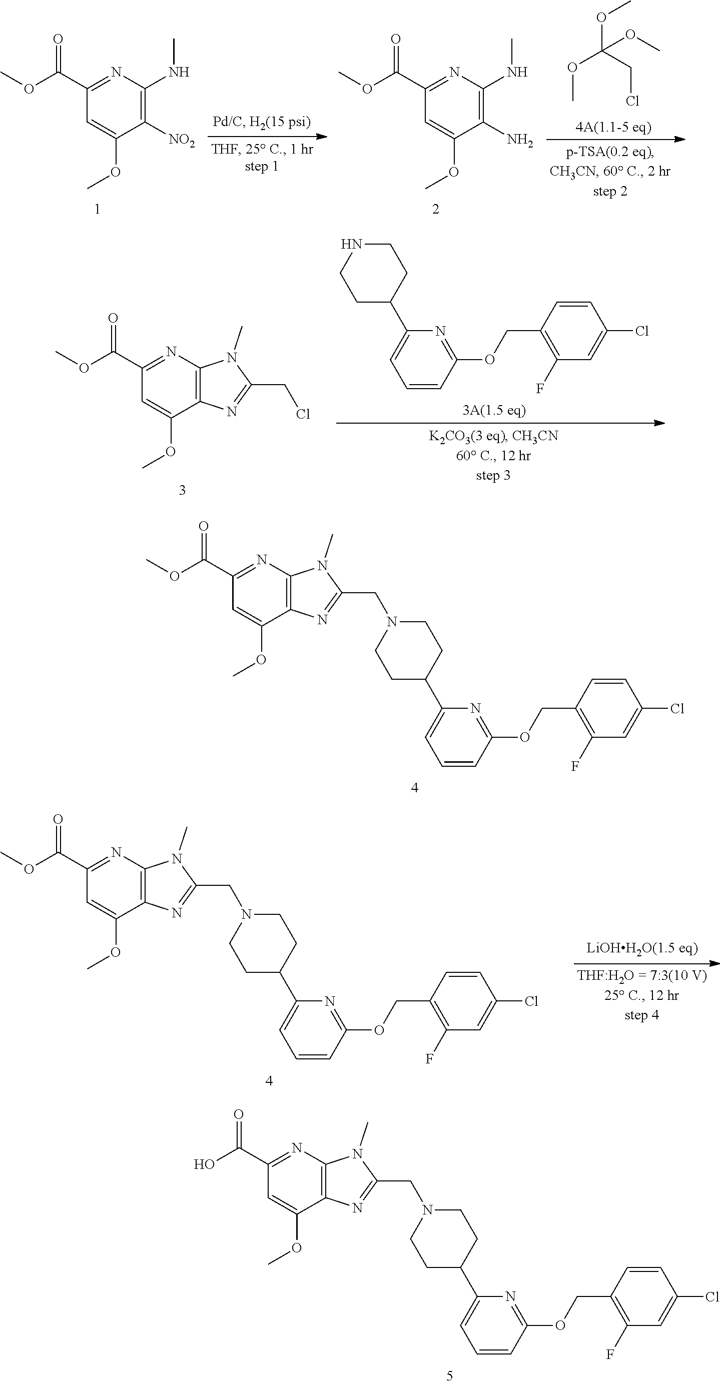

### General Procedure for Preparation of Intermediate 2:

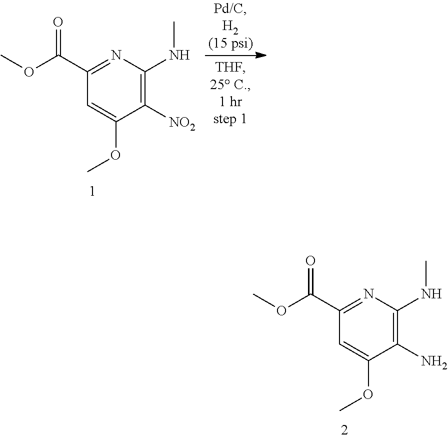 [SMILES: CNc1nc(C(=O)OC)cc(OC)c1[N+](=O)[O-] ; CNc1nc(C(=O)OC)cc(OC)c1N]

Equip a 25 mL three-necked round bottom flask, addition funnel and thermometer, H₂ balloon. THF (3 mL) was charged to the three-necked round bottom flask, then 1 (150 mg, 621.89 μmol, 1 eq) was added to the mixture at 15° C. At 15° C. inner temperature, Pd/C (198.54 mg) was added in portions to the reaction mixture at 15° C. After the addition, the mixture was stirred at 15° C. for 2 hr. LCMS showed 1 was consumed completely and one main peak with desired m/z was detected. The suspension was filtered and the filter cake was washed with THF (10 mL×3). The combined filtrates were concentrated to dryness to obtained 2 (120 mg, 568.14 μmol, 91.36% yield) as a brown solid.

LCMS: RT=0.986 min, MS cal.: 211.22, [M+H]⁺=212.0

¹H NMR (400 MHz, DMSO-d₆) δ=7.14 (s, 1H), 5.77 (br d, J=4.5 Hz, 1H), 4.89 (s, 2H), 3.82 (s, 3H), 3.75 (s, 3H), 2.86 (d, J=4.5 Hz, 3H)

### General Procedure for Preparation of Intermediate 3:

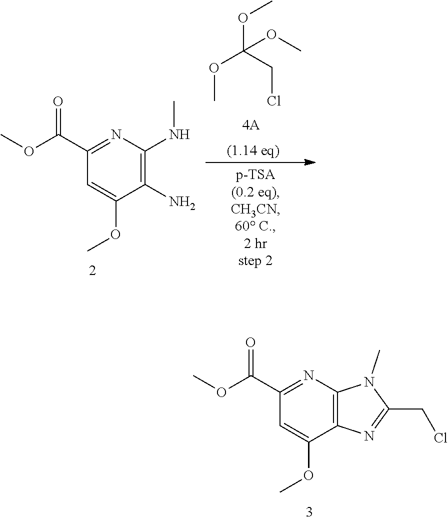

To a solution of 2 (120 mg, 568.14 μmol, 1 eq) in ACN (2 mL) was added 4A (175.66 mg, 1.14 mmol, 153.15 μL, 2 eq) and TsOH (19.57 mg, 113.63 μmol, 0.2 eq) at 15° C., the mixture was stirred at 60° C. for 2 hr. LCMS showed Compound 2 was consumed completely and one main peak with desired m/z was detected. The reaction mixture was concentrated under reduced pressure to give a residue, and then diluted with H₂O (5 mL) and extracted with DCM (5 mL*3). The combined organic layers were washed with brine (5 mL*2), dried over Na₂SO₄, filtered, and concentrated under reduced pressure to give a residue. The crude product was triturated with MTBE (10 V) at 10° C. for 15 min to obtain Compound 3 (174 mg, crude) as a black solid.

LCMS: RT=1.185 min, MS cal.: 269.69, [M+H]⁺=269.9

¹H NMR (400 MHz, CHCl3-d) δ=7.65 (s, 1H), 4.85 (s, 2H), 4.17 (s, 3H), 4.03 (d, J=11.0 Hz, 6H)

### General Procedure for Preparation of Intermediate 4:

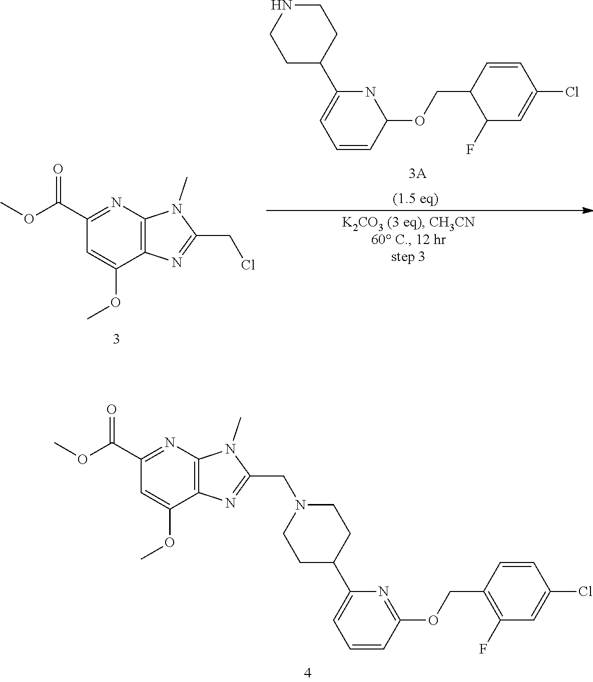

To a solution of 3 (60 mg, 222.48 μmol, 1 eq) in ACN (2 mL) was added 3A (78.51 mg, 244.73 μmol, 1.1 eq) and K₂CO₃ (92.25 mg, 667.45 μmol, 3 eq) at 15° C., the mixture was stirred at 60° C. for 12 hr. LCMS showed 3 was consumed completely and one main peak with desired m/z was detected. The reaction mixture was diluted with H₂O (5 mL) and extracted with EtOAc (5 mL*3). The combined organic layers were washed with brine (5 mL*2), dried over Na₂SO₄, filtered and concentrated under reduced pressure to give a residue. The residue was purified by column chromatography (SiO₂, Petroleum ether/Ethyl acetate=1/0 to 1/1) to obtained Compound 4 (90 mg, 162.45 μmol, 73.02% yield) as a white solid.

LCMS: RT=2.159 min, MS cal.: 554.02, [M+H]⁺=554.1

¹H NMR (400 MHz, CHCl3-d) δ=7.66-7.61 (m, 1H), 7.54-7.41 (m, 2H), 7.15-7.08 (m, 2H), 6.73 (br d, J=7.2 Hz, 1H), 6.61 (br d, J=8.1 Hz, 1H), 5.40 (s, 2H), 4.18-4.13 (m, 3H), 4.10-4.02 (m, 6H), 3.89 (br s, 2H), 3.04-2.87 (m, 2H), 2.69-2.51 (m, 1H), 2.39-2.21 (m, 2H), 1.96-1.71 (m, 4H)

### General Procedure for Preparation of Compound 323 (5):

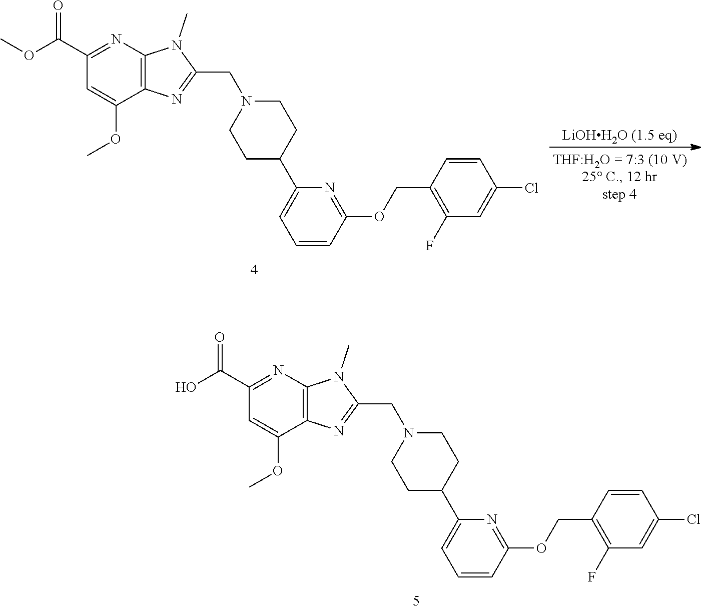 [SMILES: COC(=O)c1cc(OC)c2nc(CN3CCC(c4cccc(OCc5ccc(Cl)cc5F)n4)CC3)n(C)c2n1 ; COc1cc(C(=O)O)nc2c1nc(CN1CCC(c3cccc(OCc4ccc(Cl)cc4F)n3)CC1)n2C]

To a solution of Compound 4 (80 mg, 144.40 μmol, 1 eq) in THF (0.7 mL) and H₂O (0.3 mL) was added LiOH·H₂O (9.09 mg, 216.60 μmol, 1.5 eq), the mixture was stirred at 25° C. for 12 hr. LCMS showed 4 was consumed completely and one main peak with desired m/z was detected. The mixture was filtered and purified by prep-HPLC (neutral condition; column: Waters Xbridge Prep OBD C18 150*40 mm*10 um; mobile phase: [H₂O (10 mM NH₄HCO₃)-ACN]; gradient: 20%-70% B over 8.0 min) to obtained 2-((4-(6-((4-Chloro-2-fluorobenzyl)oxy)pyridin-2-yl)piperidin-1-yl)methyl)-7-methoxy-3-methyl-3H-imidazo[4,5-b]pyridine-5-carboxylic acid (28.85 mg, 53.43 μmol, 37.00% yield) as a white solid.

LCMS: RT=1.114 min, MS cal.: 539.99, [M+H]⁺=540.0

¹H NMR (400 MHz, MeOH-d4) δ=7.68 (s, 1H), 7.61-7.55 (m, 1H), 7.49 (t, J=8.1 Hz, 1H), 7.23-7.15 (m, 2H), 6.83 (d, J=7.2 Hz, 1H), 6.64 (d, J=8.2 Hz, 1H), 5.41 (s, 2H), 4.15 (s, 3H), 4.05 (s, 2H), 4.03 (s, 3H), 3.18 (br d, J=11.2 Hz, 2H), 2.77-2.64 (m, 1H), 2.58-2.44 (m, 2H), 1.98-1.85 (m, 4H)

### Example A61—Preparation of Compound 325. 2-((4-(6-((4-Chloro-2-fluorobenzyl)oxy)pyridin-2-yl)piperidin-1-yl)methyl)-4-((1r,3r)-3-methoxycyclobutoxy)-1-methyl-1H-benzo[d]imidazole-6-carboxylic acid (Compound 325) (3)

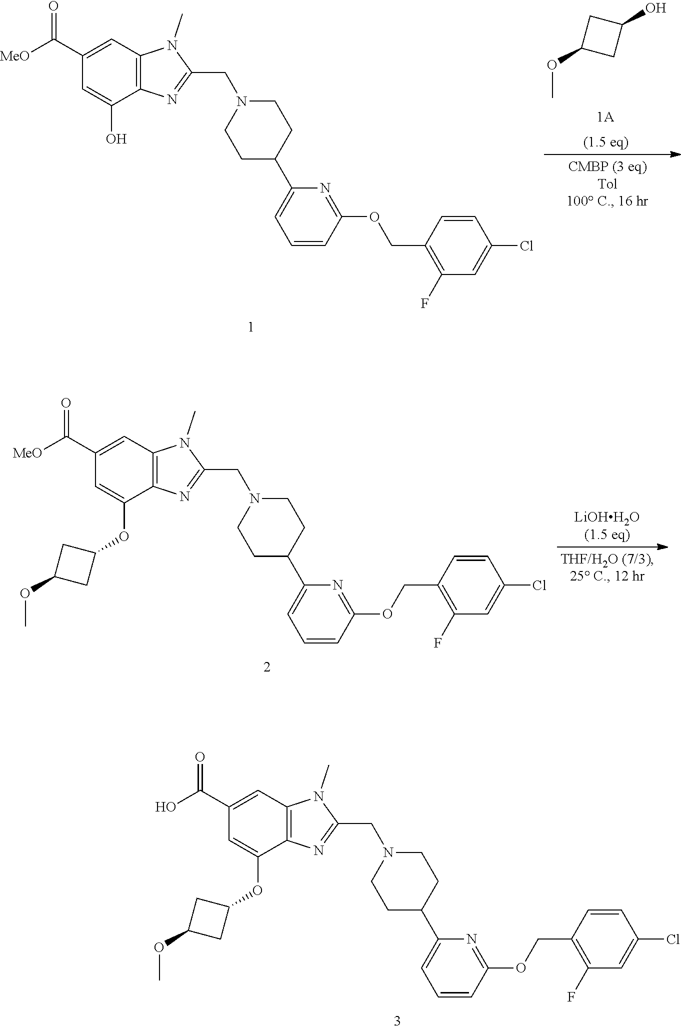

### General Procedure for Preparation of Intermediate 2:

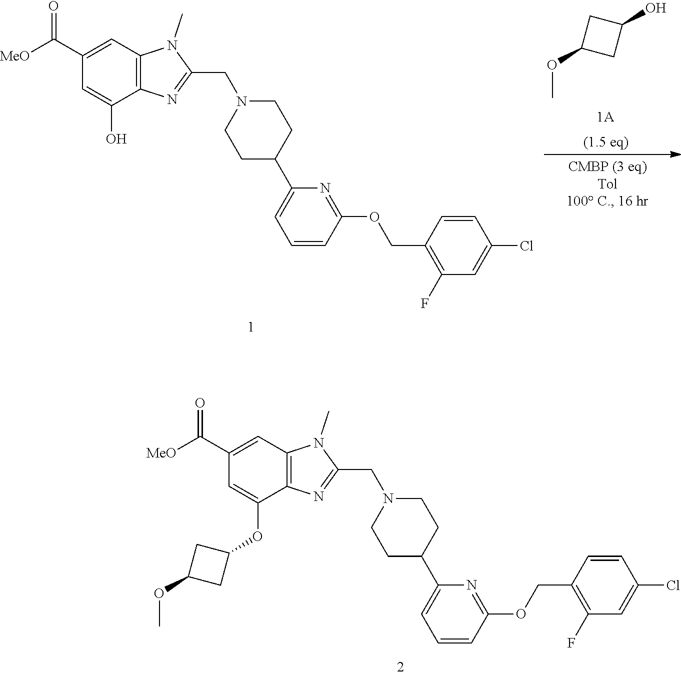

A mixture of 1 (100 mg, 185.53 μmol, 1 eq), 1A (28.42 mg, 278.29 μmol, 1.5 eq), CMBP acetonitrile (134.33 mg, 556.59 μmol, 3 eq) in Toluene (5 mL) was degassed and purged with N₂ for 3 times, and then the mixture was stirred at 100° C. for 16 hr under N₂ atmosphere. LC-MS (RT=1.691 min) showed 1 was consumed completely and one main peak with desired mass was detected. The reaction mixture was diluted with H₂O 15 mL and extracted with EtOAc 60 mL (20 mL*3). The combined organic layers were washed with brine 15 mL, dried over Na₂SO₄, filtered and concentrated under reduced pressure to give a residue. The residue was purified by column chromatography (SiO₂, Petroleum ether/Ethyl acetate=10/1 to 0/1). 2 (90 mg, 144.44 μmol, 77.85% yield) was obtained as a yellow oil.

LCMS: RT=1.691 min, MS cal.: 622.24, [M+H]⁺=623.3

SFC: ee %=100%

¹H NMR (400 MHz, CHCl3-d) δ=7.77 (s, 1H), 7.53-7.41 (m, 2H), 7.20 (s, 1H), 7.14-7.08 (m, 2H), 6.74 (br d, J=7.2 Hz, 1H), 6.61 (br d, J=7.6 Hz, 1H), 5.41 (s, 2H), 5.18-5.10 (m, 1H), 4.07-3.87 (m, 7H), 3.30 (s, 3H), 3.04-2.90 (m, 2H), 2.71-2.53 (m, 5H), 2.39-2.20 (m, 2H), 1.94-1.79 (m, 3H), 1.69-1.53 (m, 3H)

### General Procedure for Preparation of Compound 325 (3):

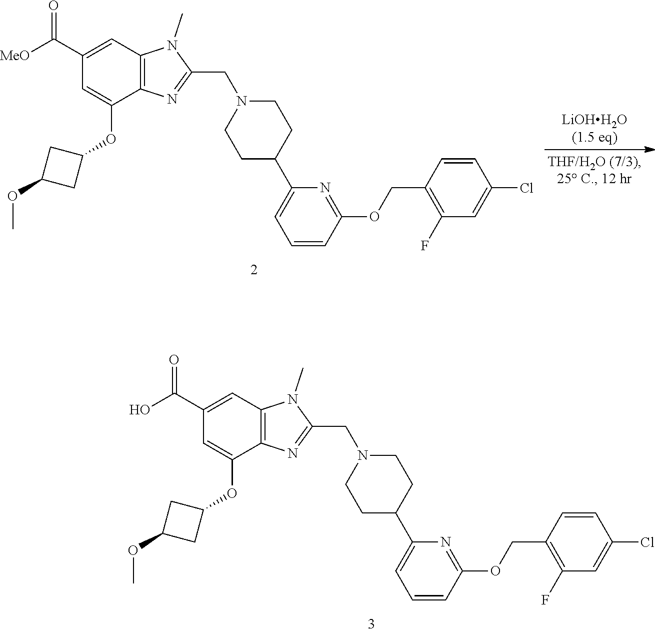 [SMILES: COC(=O)c1cc(O[C@H]2C[C@H](OC)C2)c2nc(CN3CCC(c4cccc(OCc5ccc(Cl)cc5F)n4)CC3)n(C)c2c1 ; CO[C@H]1C[C@H](Oc2cc(C(=O)O)cc3c2nc(CN2CCC(c4cccc(OCc5ccc(Cl)cc5F)n4)CC2)n3C)C1]

A mixture of 2 (75 mg, 120.36 μmol, 1 eq), LiOH·H₂O (7.58 mg, 180.55 μmol, 1.5 eq) and in THF (0.7 mL) and H₂O (0.3 mL) was degassed and purged with N₂ for 3 times, and then the mixture was stirred at 25° C. for 12 hr under N₂ atmosphere. LC-MS (product, RT=1.199 min) showed 2 was consumed completely and one main peak with desired mass was detected. The reaction was purified directly. The residue was purified by prep-HPLC (column: Waters Xbridge Prep OBD C18 150*40 mm*10 um; mobile phase: [H₂O (10 mM NH₄HCO₃)-ACN]; gradient: 25%-65% B over 8.0 min). 2-((4-(6-((4-chloro-2-fluorobenzyl)oxy)pyridin-2-yl)piperidin-1-yl)methyl)-4-((1r,3r)-3-methoxycyclobutoxy)-1-methyl-1H-benzo[d]imidazole-6-carboxylic acid (25.62 mg, 41.74 μmol, 34.68% yield, 99.23% purity) was obtained as a white solid.

LCMS: RT=2.334 min, MS cal.: 608.2, [M+1]⁺=609.2

HPLC: RT=11.90 min, purity: 99.28%

SFC: ee %=100%

¹H NMR (400 MHz, MeOH-d4) δ=7.86 (d, J=1.2 Hz, 1H), 7.57 (t, J=7.6 Hz, 1H), 7.48 (t, J=7.6 Hz, 1H), 7.25-7.15 (m, 3H), 6.82 (d, J=7.2 Hz, 1H), 6.63 (d, J=8.4 Hz, 1H), 5.40 (s, 2H), 5.08 (t, J=5.2 Hz, 1H), 4.21 (t, J=5.6 Hz, 1H), 3.98 (s, 3H), 3.94 (s, 2H), 3.29 (s, 3H), 3.07 (br d, J=11.2 Hz, 2H), 2.66 (br t, J=7.2 Hz, 1H), 2.55 (t, J=5.6 Hz, 4H), 2.44-2.33 (m, 2H), 1.94-1.83 (m, 4H)

### Example A62—Preparation of Compound 326 and Compound 300. (S)-2-((4-(2-(4-Cyano-2-fluorophenyl)-2,3-dihydrobenzo[b][1,4]dioxin-5-yl)piperidin-1-yl)methyl)-4-(difluoromethoxy)-1-methyl-1H-benzo[d]imidazole-6-carboxylic acid (Compound 326) & (R)-2-((4-(2-(4-Cyano-2-fluorophenyl)-2,3-dihydrobenzo[b][1,4]dioxin-5-yl)piperidin-1-yl)methyl)-4-(difluoromethoxy)-1-methyl-1H-benzo[d]imidazole-6-carboxylic acid (Compound 300)

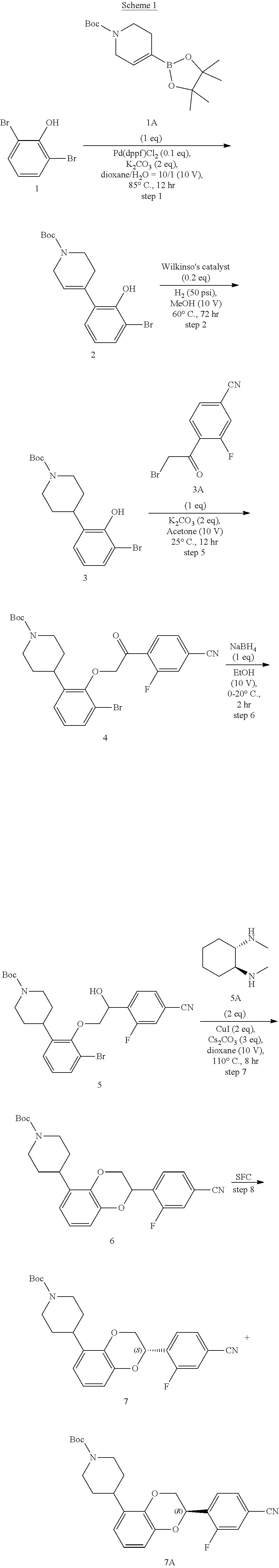

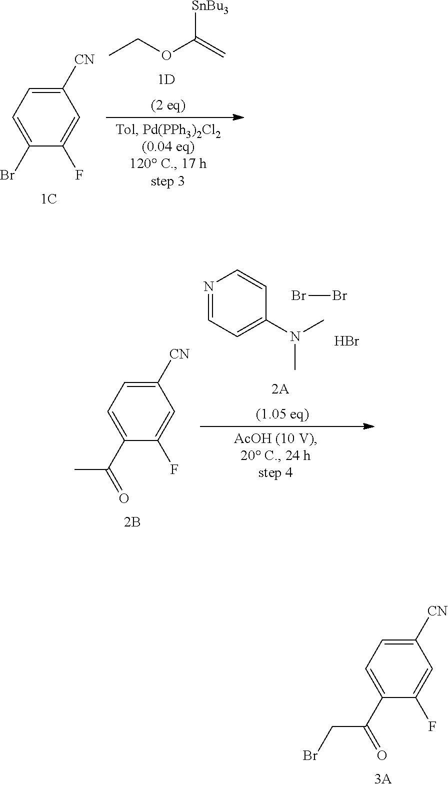

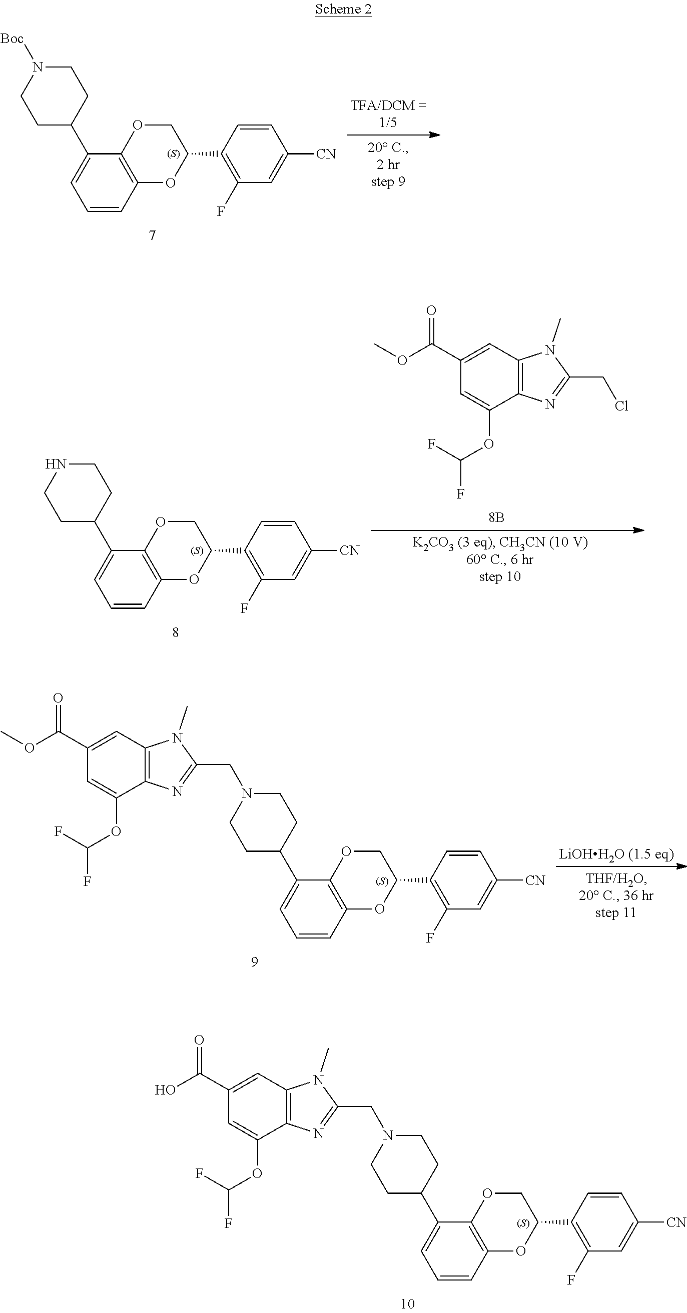

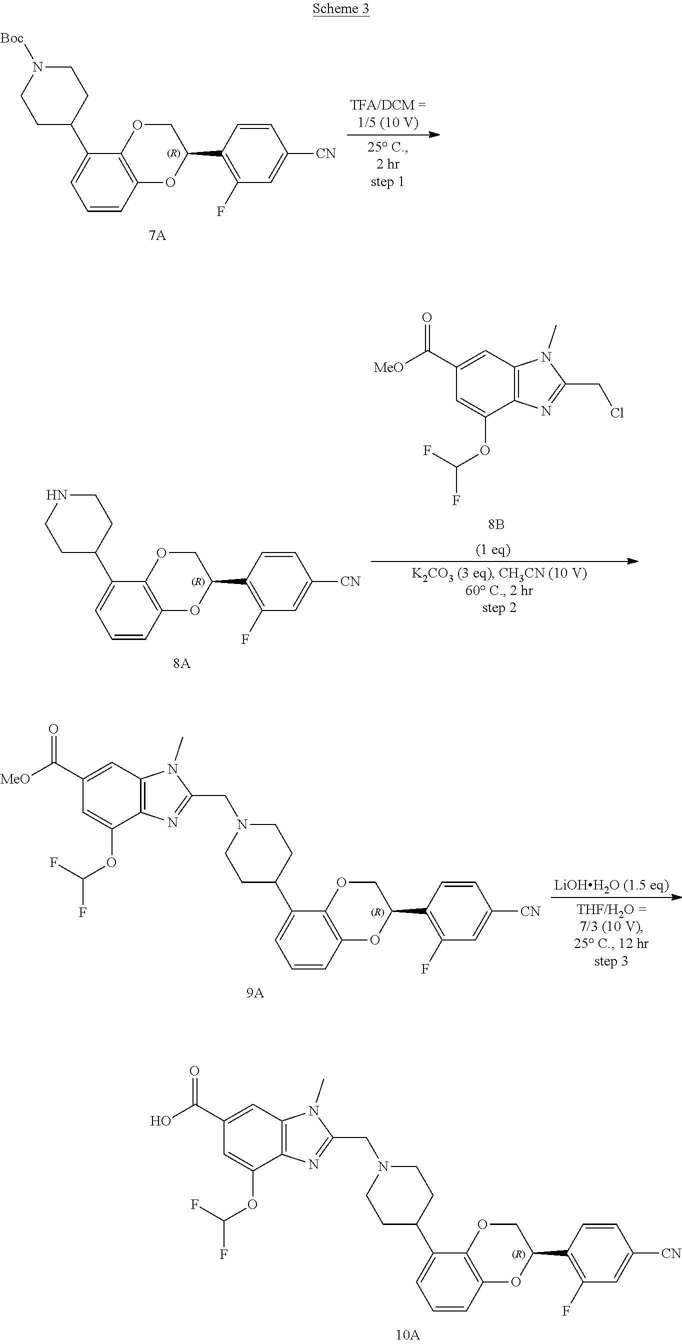

### General Procedure for Preparation of Scheme 1, Intermediate 2:

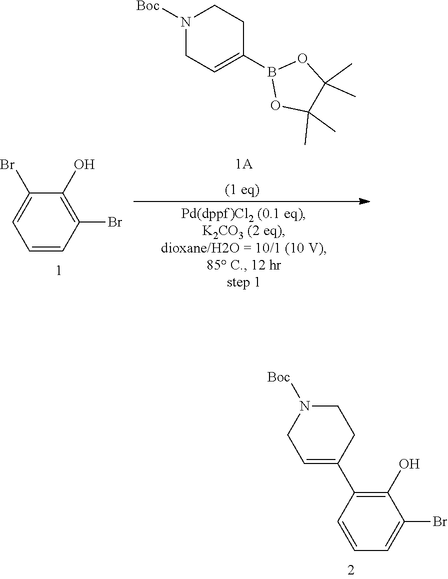 [SMILES: CC(C)(C)OC(=O)N1CC=C(B2OC(C)(C)C(C)(C)O2)CC1 ; Oc1c(Br)cccc1Br ; CC(C)(C)OC(=O)N1CC=C(c2cccc(Br)c2O)CC1]

A mixture of 1 (17 g, 67.49 mmol, 1 eq), 1A (20.87 g, 67.49 mmol, 1 eq), Pd(dppf)Cl₂ (4.94 g, 6.75 mmol, 0.1 eq),K₂CO₃ (18.65 g, 134.97 mmol, 2 eq) in dioxane (150 mL) and H₂O (20 mL) was degassed and purged with N₂ for 3 times, and then the mixture was stirred at 85° C. for 12 hr under N₂ atmosphere. LCMS showed 1 was consumed completely and one main peak with desired m/z was detected. The reaction mixture was diluted with H₂O 200 mL and extracted with EtOAc 300 mL (100 mL*3), dried over Na₂SO₄, filtered and concentrated under reduced pressure to give a residue. The residue was purified by column chromatography (SiO₂, Petroleum ether/Ethyl acetate=100/1 to 94/6). 2 (6.5 g, 17.43 mmol, 25.83% yield) was obtained as a white solid.

LCMS: RT=0.593 min, MS cal.: 353.1/355.1, [M−100]⁺=253.9/260.1

### General Procedure for Preparation of Scheme 1, Intermediate 3:

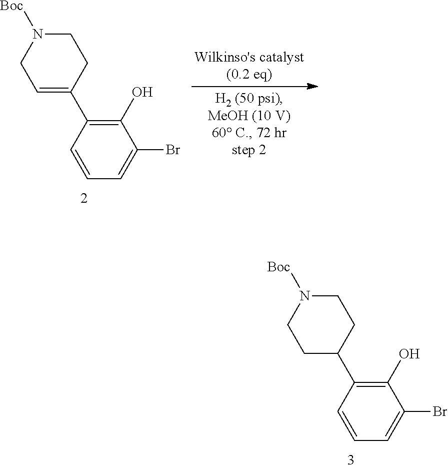 [SMILES: CC(C)(C)OC(=O)N1CC=C(c2cccc(Br)c2O)CC1 ; CC(C)(C)OC(=O)N1CCC(c2cccc(Br)c2O)CC1]

Equip a 250 mL necked round bottom flask, addition funnel, H₂ balloon etc. MeOH (60 mL) was charged to the 250 mL necked round bottom flask, then chlororhodium; triphenylphosphane (3.13 g, 3.39 mmol, 0.2 eq) was added at 25° C. within 1 min. At 25° C., 2 (6 g, 16.94 mmol, 1 eq) in MeOH (60 mL) was added to the reaction mixture at 25° C., within 2 min under H₂ atmosphere. After the addition, the mixture was stirred at 60° C. for 72 hr under H₂ atmosphere (50Psi). HNMR revealed that 20% of 1 remained. After 72 hr. the reaction mixture was filtered and concentrated under reduced pressure to give a residue. The residue was purified by column chromatography (SiO₂, Petroleum ether/Ethyl acetate=100/1 to 10/1). 3 (3.4 g, 8.40 mmol, 49.58% yield) was obtained as a gray solid.

LCMS: RT=0.853 min, MS cal.: 355.1/357.1, [M−55]⁺=300.0/302.0

### General Procedure for Preparation of Scheme 1, Intermediate 2B:

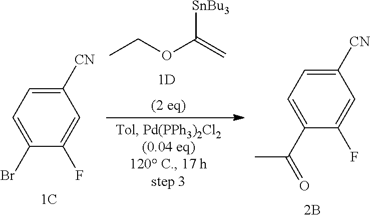

To a solution of 1C (25 g, 125.00 mmol, 1 eq) in Toluene (375 mL) was added 1D (49.66 g, 137.49 mmol, 46.45 mL, 1.1 eq) and Pd(PPh₃)₂Cl₂ (3.51 g, 5.00 mmol, 0.04 eq) at 20° C. The mixture was stirred for 5 hr at 120° C. under N₂ and then added 1D (40.63 g, 112.50 mmol, 38.01 mL, 0.9 eq) at 20° C. and stirred for 12 hr at 120° C. under N₂. HPLC showed that the starting material was consumed. The mixture was cooled down to 20° C. and added KF (250 ml, 2M) at 20° C. The mixture was stirred for 30 min and then added HCl (500 ml, 6M)) and stirred for 2 hr at 70° C. under N₂. The mixture was extracted with EA (800 ml, 500 ml) and washed with brine (300 ml) and concentrated to give the residue. The crude product was purified by column chromatography (SiO₂, PE/EA=100:1˜3:1). 2B (18 g, 110.33 mmol, 88.27% yield) was obtain as a white solid.

¹H NMR (400 MHz, DMSO-d6) δ=8.09 (dd, J=1.2, 10.0 Hz, 1H), 8.01 (t, J=8.0 Hz, 1H), 7.87 (dd, J=1.2, 8.0 Hz, 1H), 2.67 (d, J=4.0 Hz, 3H)

### General Procedure for Preparation of Scheme 1, Intermediate 3A:

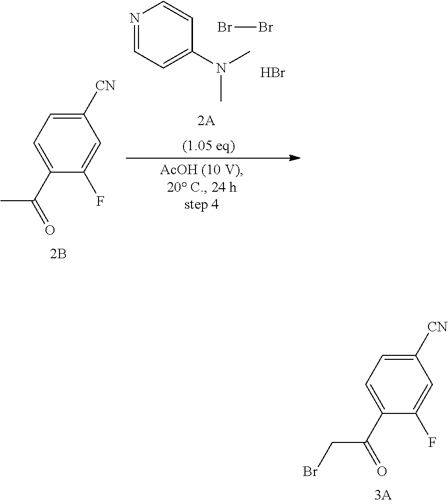

To a solution of 2B (10 g, 61.29 mmol, 1 eq) in AcOH (100 mL) was added 2A (23.35 g, 64.36 mmol, 1.05 eq) at 20° C. The mixture was stirred for 24 hr at 20° C. under N₂. HPLC showed that the starting material was consumed. The mixture was filtered to give the residue. The crude product was triturated with water (50 ml) and filtered to give the filter cake. 3A (14.2 g, 58.67 mmol, 53.17% yield) was obtain as a white solid.

¹H NMR (400 MHz, DMSO-d6) δ=8.14-8.02 (m, 2H), 7.88 (dd, J=1.2, 8.0 Hz, 1H), 4.90 (d, J=2.0 Hz, 2H)

### General Procedure for Preparation of Scheme 1, Intermediate 4:

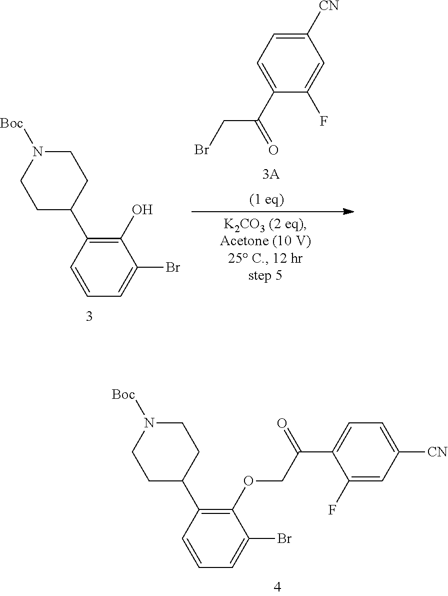 [SMILES: CC(C)(C)OC(=O)N1CCC(c2cccc(Br)c2O)CC1 ; N#Cc1ccc(C(=O)CBr)c(F)c1 ; CC(C)(C)OC(=O)N1CCC(c2cccc(Br)c2OCC(=O)c2ccc(C#N)cc2F)CC1]

A mixture of 3 (200 mg, 561.40 μmol, 1 eq), 3A (163.06 mg, 673.68 μmol, 1.2 eq), K₂CO₃ (155.18 mg, 1.12 mmol, 2 eq) in Acetone (2 mL) was degassed and purged with N₂ for 3 times, and then the mixture was stirred at 25° C. for 12 hr under N₂ atmosphere. LCMS (RT=1.606 min) showed 3 was consumed completely and one main peak with desired mass was detected. The reaction mixture was diluted with H₂O 10 mL and extracted with EtOAc (10 mL*3). The combined organic layers were dried over Na₂SO₄, filtered, and concentrated under reduced pressure to give a residue. The residue was purified by column chromatography (SiO₂, Petroleum ether/Ethyl acetate=100/1 to 0/1). 4 (870 mg, 1.68 mmol, 74% yield) was obtained as a yellow solid.

LCMS: RT=1.612 min, MS cal.: 516.1, [M−100]⁺=416.9

¹H NMR (400 MHz, CHCl3-d) δ=8.18 (dd, J=7.2, 8.0 Hz, 1H), 7.64 (dd, J=1.6, 8.0 Hz, 1H), 7.52 (dd, J=1.2, 10.0 Hz, 1H), 7.44 (dd, J=1.6, 8.0 Hz, 1H), 7.18 (dd, J=1.6, 7.6 Hz, 1H), 7.10-7.00 (m, 1H), 5.19 (d, J=3.2 Hz, 2H), 4.24 (d, J=13.2 Hz, 2H), 3.28-3.15 (m, 1H), 2.89-2.73 (m, 2H), 1.77 (d, J=12.4 Hz, 2H), 1.66-1.57 (m, 2H), 1.49 (s, 9H)

### General Procedure for Preparation of Scheme 1, Intermediate 5:

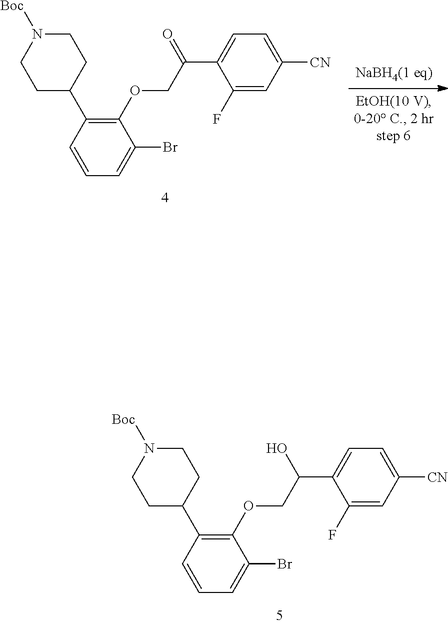 [SMILES: CC(C)(C)OC(=O)N1CCC(c2cccc(Br)c2OCC(=O)c2ccc(C#N)cc2F)CC1 ; "EtOH(10 V),0-20° C., 2 hrstep 6" ; "NaBH4(1 eq)" ; CC(C)(C)OC(=O)N1CCC(c2cccc(Br)c2OCC(O)c2ccc(C#N)cc2F)CC1]

EtOH (8 mL) was charged to the three-necked round bottom flask, then 4 (870 mg, 1.68 mmol, 1 eq) was added to the mixture at 20° C. At 0° C., NaBH₄ (63.62 mg, 1.68 mmol, 1 eq) was added in portions to the reaction mixture. After the addition, the mixture was stirred at 20° C. for 2 hr. LCMS showed 4 was consumed completely and one main peak with desired mass was detected. The reaction mixture was added to NH₄Cl (20 mL) at 20° C. The mixture was extracted by EtOAc (10 mL*3). Then organic phase was combined and washed by brine (20 mL*3), dried with Na₂SO₄, filtered. The organic phase was concentrated under reduced pressure at 35° C. to give a residue. 5 (770 mg, 1.48 mmol, 88.16% yield) was obtained as a yellow solid.

LCMS: RT=1.552 min, MS cal.: 518.1, [M−100]⁺=418.9

¹H NMR (400 MHz, CHCl3-d) δ=7.86 (t, J=7.6 Hz, 1H), 7.57-7.53 (m, 1H), 7.42 (dd, J=1.2, 8.0 Hz, 1H), 7.37 (dd, J=1.2, 9.6 Hz, 1H), 7.15 (dd, J=1.6, 7.6 Hz, 1H), 7.05-6.99 (m, 1H), 5.55 (dd, J=2.4, 7.2 Hz, 1H), 4.25 (br t, J=10.4 Hz, 2H), 4.14 (dd, J=2.4, 9.2 Hz, 1H), 4.04-3.95 (m, 1H), 3.29-3.15 (m, 1H), 3.04-2.90 (m, 1H), 2.82-2.62 (m, 2H), 1.79-1.66 (m, 2H), 1.58 (br dd, J=3.2, 12.8 Hz, 2H), 1.51-1.48 (m, 9H)

### General Procedure for Preparation of Scheme 1, Intermediate 6:

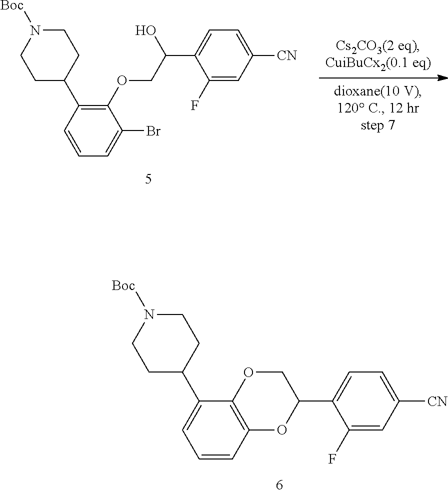 [SMILES: CC(C)(C)OC(=O)N1CCC(c2cccc(Br)c2OCC(O)c2ccc(C#N)cc2F)CC1 ; "Cs2CO3(2 eq),CuiBuCx2(0.1 eq)" ; "dioxane(10 V),120° C., 12 hrstep 7" ; CC(C)(C)OC(=O)N1CCC(c2cccc3c2OCC(c2ccc(C#N)cc2F)O3)CC1]

A mixture of 5 (300 mg, 577.59 μmol, 1 eq), Cs₂CO₃ (376.38 mg, 1.16 mmol, 2 eq), CuiBuCx₂ (22.99 mg, 57.76 μmol, 0.1 eq) in dioxane (3 mL) was degassed and purged with N₂ for 3 times, and then the mixture was stirred at 120° C. for 12 hr under N₂ atmosphere. LCMS (RT=1.665 min) showed 0% of 5 remained. Several new peaks were shown on LC-MS and 94% of desired Compound was detected. The reaction mixture was diluted with H₂O 15 mL and extracted with DCM (15 mL*3). The combined organic layers were dried over Na₂SO₄, filtered, and concentrated under reduced pressure to give a residue. The residue was purified by column chromatography (SiO₂, Petroleum ether/Ethyl acetate=100/1 to 0/1). 6 (410 mg, 935.03 μmol, 80% yield) was obtained as a white solid.

¹H NMR (400 MHz, CHCl3-d) δ=7.73-7.66 (m, 1H), 7.55 (d, J=8.0 Hz, 1H), 7.43 (d, J=9.6 Hz, 1H), 6.93-6.86 (m, 2H), 6.81 (dd, J=3.6, 5.6 Hz, 1H), 5.50 (d, J=6.4 Hz, 1H), 4.51 (dd, J=2.0, 11.2 Hz, 1H), 4.25 (d, J=12.4 Hz, 2H), 3.97 (dd, J=8.0, 11.2 Hz, 1H), 3.10-2.99 (m, 1H), 2.88-2.76 (m, 2H), 1.88-1.73 (m, 2H), 1.69-1.58 (m, 2H), 1.49 (s, 9H)

### General Procedure for Preparation of Scheme 1, Intermediate 7& 7A:

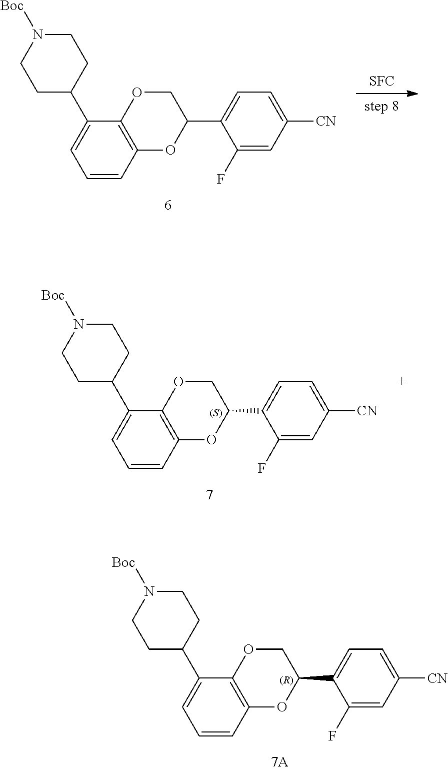

Compound 6 was separated by SFC (column: DAICEL CHIRALPAK IG 250 mm*30 mm*5 um; mobile phase: [CO₂-MeOH (0.1% NH₃H₂O)]; B %: 33%, isocratic elution mode). 7 (210 mg, 478.92 μmol, 42% yield) was obtained as a white solid.

SFC: RT=1.730 min, purity: 99.96%

¹H NMR (400 MHz, CHCl3-d) δ=7.69 (t, J=7.6 Hz, 1H), 7.55 (dd, J=1.2, 8.0 Hz, 1H), 7.43 (dd, J=1.2, 9.6 Hz, 1H), 6.93-6.88 (m, 2H), 6.85-6.77 (m, 1H), 5.50 (dd, J=1.6, 8.0 Hz, 1H), 4.51 (dd, J=2.0, 11.2 Hz, 1H), 4.24 (d, J=12.0 Hz, 2H), 3.97 (dd, J=8.0, 11.2 Hz, 1H), 3.04 (tt, J=3.6, 12.0 Hz, 1H), 2.89-2.74 (m, 2H), 1.88-1.72 (m, 2H), 1.68-1.58 (m, 2H), 1.49 (s, 9H)

7A (230 mg, 524.53 μmol, 46% yield) was obtained as a white solid.

SFC: RT=1.995 min, purity: 98.76%

¹H NMR (400 MHz, CHCl3-d) δ=7.69 (t, J=7.6 Hz, 1H), 7.60-7.51 (m, 1H), 7.43 (dd, J=1.6, 9.6 Hz, 1H), 6.93-6.87 (m, 2H), 6.85-6.78 (m, 1H), 5.55-5.45 (m, 1H), 4.51 (dd, J=2.0, 11.2 Hz, 1H), 4.24 (d, J=13.6 Hz, 2H), 3.97 (dd, J=8.0, 11.2 Hz, 1H), 3.10-2.98 (m, 1H), 2.89-2.76 (m, 2H), 1.88-1.73 (m, 2H), 1.69-1.60 (m, 2H), 1.49 (s, 9H)

### General Procedure for Preparation of Scheme 2, 8:

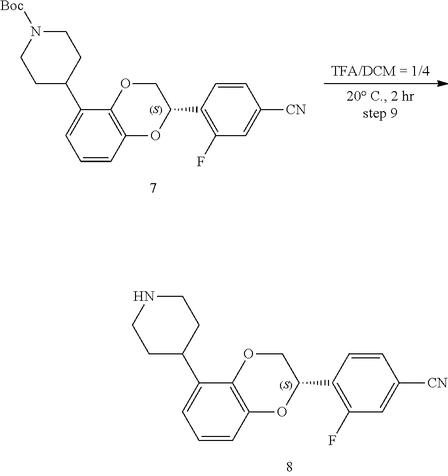

To a solution of 7 (0.16 g, 364.89 μmol, 1 eq) in DCM (4 mL) was added TFA (1 mL) at 20° C. The mixture was stirred for 2 hr at 20° C. under N₂. LCMS (ET87376-22-P1A1) showed that the starting material was consumed. The mixture was concentrated to give the residue. 8 (0.165 g, 364.72 μmol, 99.95% yield, TFA) was obtained as a white solid.

LCMS: RT=0.394 min, MS cal.: 338.3, [M+H]⁺=339.1

### General Procedure for Preparation of Scheme 2, Intermediate 9:

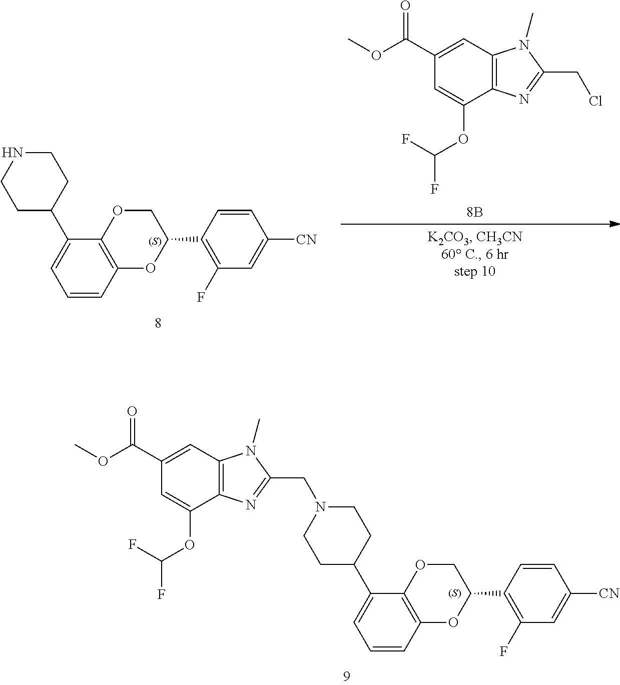

To a solution of 8 (0.165 g, 364.72 μmol, 1 eq, TFA) in ACN (5 mL) was added K₂CO₃ (151.22 mg, 1.09 mmol, 3 eq) and 8B (111.12 mg, 364.72 μmol, 1 eq) at 20° C. The mixture was stirred for 6 hr at 60° C. under N₂. TLC (PE/EA=1:1, Rf=0.3) showed that the starting material was consumed. The mixture was poured into water (20 ml) and extract with EA (30 ml, 10 ml). The combined organic phase was washed with brine (10 ml) and concentrated to give the residue. The crude product was purified by column chromatography (SiO₂, PE/EA=100:1, 1:1). 9 (0.2 g, 329.71 μmol, 90.40% yield) was obtain as a white solid.

LCMS: RT=0.696 min, MS cal.: 606.6, [M+H]⁺=607.2

SFC: RT=1.539 min, ee %=100%

¹H NMR (400 MHz, DMSO-d6) ¹H NMR (400 MHz, DMSO-d6) δ=8.12 (s, 1H), 7.98 (d, J=10.1 Hz, 1H), 7.80 (d, J=8.0 Hz, 1H), 7.70 (t, J=7.6 Hz, 1H), 7.65 (t, J=74.0 Hz, 1H), 7.54 (s, 1H), 6.90-6.78 (m, 3H), 5.59-5.53 (m, 1H), 4.59-4.52 (m, 1H), 4.15 (dd, J=7.9, 11.4 Hz, 1H), 3.98 (s, 3H), 3.91 (s, 3H), 3.89-3.84 (m, 2H), 2.96 (br d, J=9.8 Hz, 2H), 2.91-2.82 (m, 1H), 2.30-2.16 (m, 2H), 1.79-1.57 (m, 4H)

### General Procedure for Preparation of Scheme 2, Compound 326 (10):

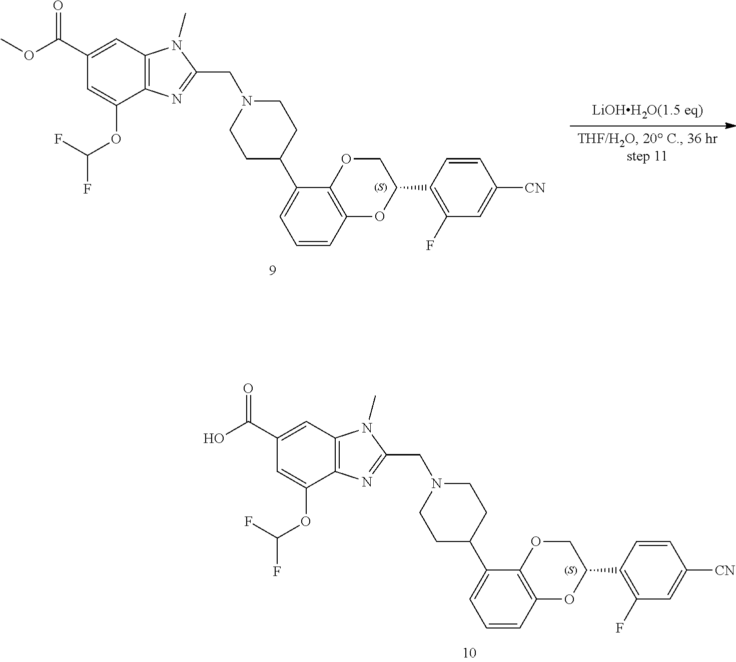

To a solution of 9 (0.05 g, 82.43 μmol, 1 eq) in THF (0.5 mL) was added LiOH·H₂O (5.19 mg, 123.64 μmol, 1.5 eq) in H₂O (0.5 mL) at 20° C. The mixture was stirred for 36 hr at 20° C. under N₂. LCMS (ET87376-28-P1H2) showed that the starting material was consumed. The mixture was diluted with THF (0.5 ml) and purified by prep-HPLC (column: Waters Xbridge Prep OBD C18 150*40 mm*10 um; mobile phase: [H₂O (10 mM NH₄HCO₃)-ACN]; gradient: 25%-55% B over 8.0 min). (S)-2-((4-(2-(4-Cyano-2-fluorophenyl)-2,3-dihydrobenzo[b][1,4]dioxin-5-yl)piperidin-1-yl)methyl)-4-(difluoromethoxy)-1-methyl-1H-benzo[d]imidazole-6-carboxylic acid (0.023 g, 38.81 μmol, 47.09% yield) was obtain as a white solid.

LCMS: RT=1.104 min, MS cal.: 592.6, [M+H]⁺=593.3

SFC: RT=3.381 min, ee %=99.74%

¹H NMR (400 MHz, DMSO-d6) δ=8.05 (s, 1H), 7.98 (dd, J=1.2, 10.0 Hz, 1H), 7.78-7.81 (m, 1H), 7.73-7.67 (m, 1H), 7.62 (t, J=74 Hz, 1H), 7.51 (s, 1H), 6.89-6.79 (m, 3H), 5.55 (dd, J=2.2, 7.8 Hz, 1H), 4.60-4.51 (m, 1H), 4.14 (br d, J=11.6 Hz, 1H), 3.95 (s, 3H), 3.84 (s, 2H), 2.95 (br d, J=9.6 Hz, 2H), 2.90-2.81 (m, 1H), 2.27-2.14 (m, 2H), 1.78-1.55 (m, 4H)

### General Procedure for Preparation of Scheme 2, Intermediate 8A:

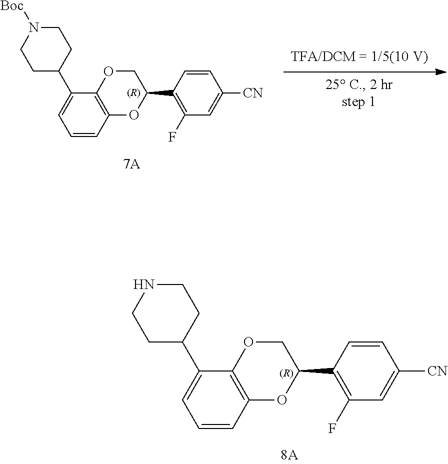

To a solution of 7A (110.00 mg, 250.86 μmol, 1 eq) in DCM (1 mL) was added TFA (337.70 mg, 2.96 mmol, 220.00 L, 11.81 eq). The mixture was stirred at 25° C. for 2 hr. LCMS showed 7A was consumed completely and one main peak with desired mass was detected. The mixture was concentrated under reduced pressure. Without purification. 8A (110 mg, crude, TFA) was obtained as a yellow oil.

LCMS: RT=0.369 min, MS cal.: 338.1/339.1, [M+1]⁺=339.1/340.1

### General Procedure for Preparation of Scheme 2, Intermediate 9A:

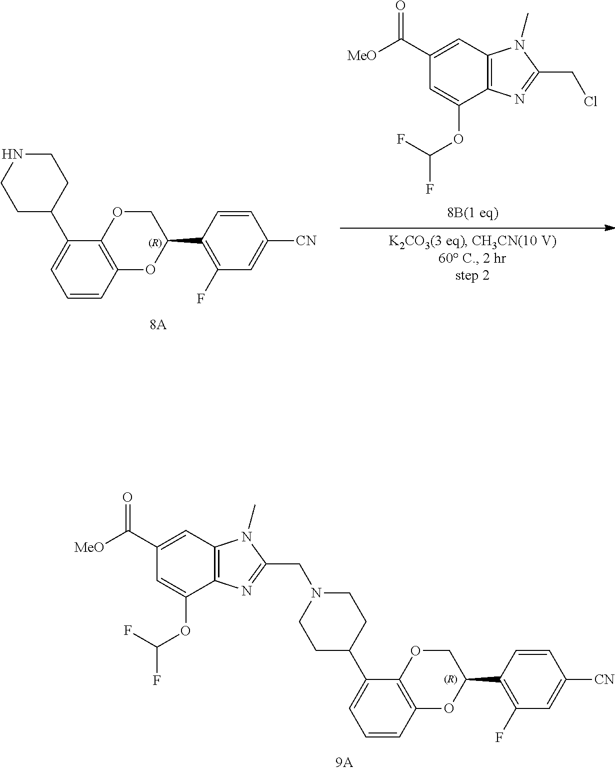

To a solution of 8A (110 mg, 243.15 μmol, 1 eq, TFA) in ACN (1 mL) was added K₂CO₃ (100.81 mg, 729.45 μmol, 3 eq) and 8B (74.08 mg, 243.15 μmol, 1 eq). The mixture was stirred at 60° C. for 2 hr. LCMS showed 8A was consumed completely and one main peak with desired mass was detected. The reaction mixture was diluted with H₂O 5 mL and extracted with EtOAc 30 mL (10 mL*3). The combined organic layers were washed with brine 10 mL, dried over Na₂SO₄, filtered and concentrated under reduced pressure to give a residue. The residue was purified by column chromatography (SiO₂, PE/EtOAc=1/0 to 1/1). 9A (80 mg, 129.25 μmol, 53.16% yield) was obtained as a white solid.

LCMS: RT=0.478 min, MS cal.: 606.2/607.2, [M+1]⁺=607.2/608.2

¹HNMR (400 MHz, CHCl3-d) δ=8.01-7.98 (m, 1H), 7.74-7.66 (m, 2H), 7.54 (d, J=8.0 Hz, 1H), 7.43 (dd, J=1.2, 9.6 Hz, 1H), 7.31 (t, J=74.8 Hz, 1H), 6.91-6.82 (m, 3H), 5.51-5.46 (m, 1H), 4.51 (dd, J=2.2, 11.2 Hz, 1H), 4.00 (br d, J=4.4 Hz, 2H), 3.98-3.97 (m, 3H), 3.89 (br s, 1H), 3.02-2.85 (m, 2H), 2.43-2.22 (m, 2H), 1.96-1.64 (m, 4H), 1.57 (br s, 2H)

SFC: RT=1.370 min, ee %=98.93%

### General Procedure for Preparation of Scheme 2, Compound 300 (10A):

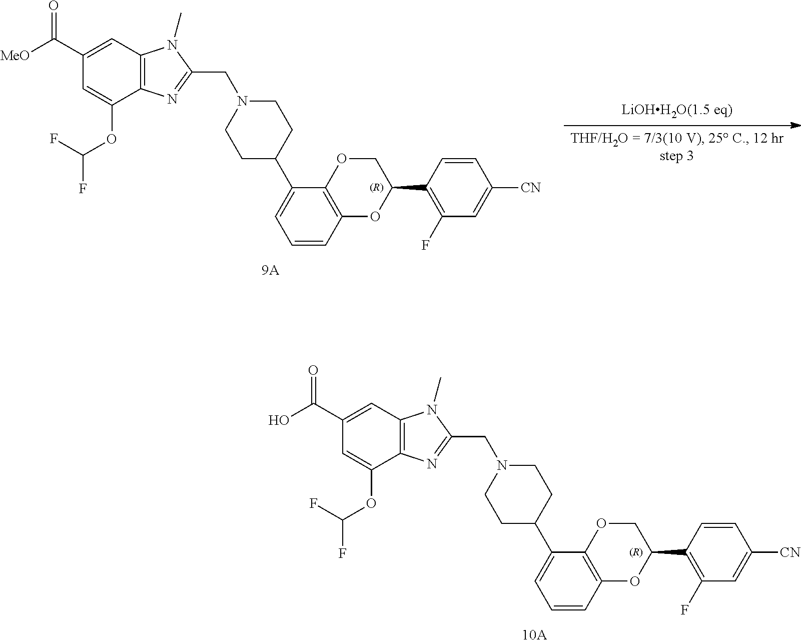

To a solution of 9A (70 mg, 115.40 μmol, 1 eq) in THF (0.7 mL) and H₂O (0.3 mL) was added LiOH·H₂O (7.26 mg, 173.10 μmol, 1.5 eq). The mixture was stirred at 25° C. for 12 hr. LCMS showed 9A was consumed completely and one main peak with desired mass was detected. The mixture was added THF 0.1 mL. The residue was purified by prep-HPLC (neutral condition: column: Waters Xbridge Prep OBD C18 150*40 mm*10 um; mobile phase: [H₂O (10 mM NH₄HCO₃)-ACN]; gradient: 25%-65% B over 8.0 min). (R)-2-((4-(2-(4-Cyano-2-fluorophenyl)-2,3-dihydrobenzo[b][1,4]dioxin-5-yl)piperidin-1-yl)methyl)-4-(difluoromethoxy)-1-methyl-1H-benzo[d]imidazole-6-carboxylic acid (28.55 mg, 47.22 μmol, 40.92% yield) was obtained as a white solid

LCMS: RT=2.673 min, MS cal.: 592.2/593.2, [M+H]⁺=593.2/594.2

HPLC: RT=11.324 min

¹HNMR (400 MHz, MeOH-d4) δ=8.14-8.09 (m, 1H), 7.74-7.69 (m, 1H), 7.69-7.62 (m, 3H), 7.25 (t, J=74.4 Hz, 1H), 6.87-6.82 (m, 3H), 5.51 (dd, J=2.1, 7.6 Hz, 1H), 4.53 (dd, J=2.4, 11.2 Hz, 1H), 4.06 (dd, J=7.8, 11.6 Hz, 1H), 4.01 (s, 3H), 4.00-3.97 (m, 2H), 3.15-3.07 (m, 2H), 3.04-2.94 (m, 1H), 2.47-2.37 (m, 2H), 1.92-1.76 (m, 4H)

SFC: RT=2.260 min, ee %=99.27%

### Example A63-Preparation of Compound 327. 2-((4-(6-((4-Cyano-2,5-difluorobenzyl)oxy)pyridin-2-yl)piperidin-1-yl)methyl)-4-methoxy-1-methyl-1H-benzo[d]imidazole-6-carboxylic acid (Compound 327)

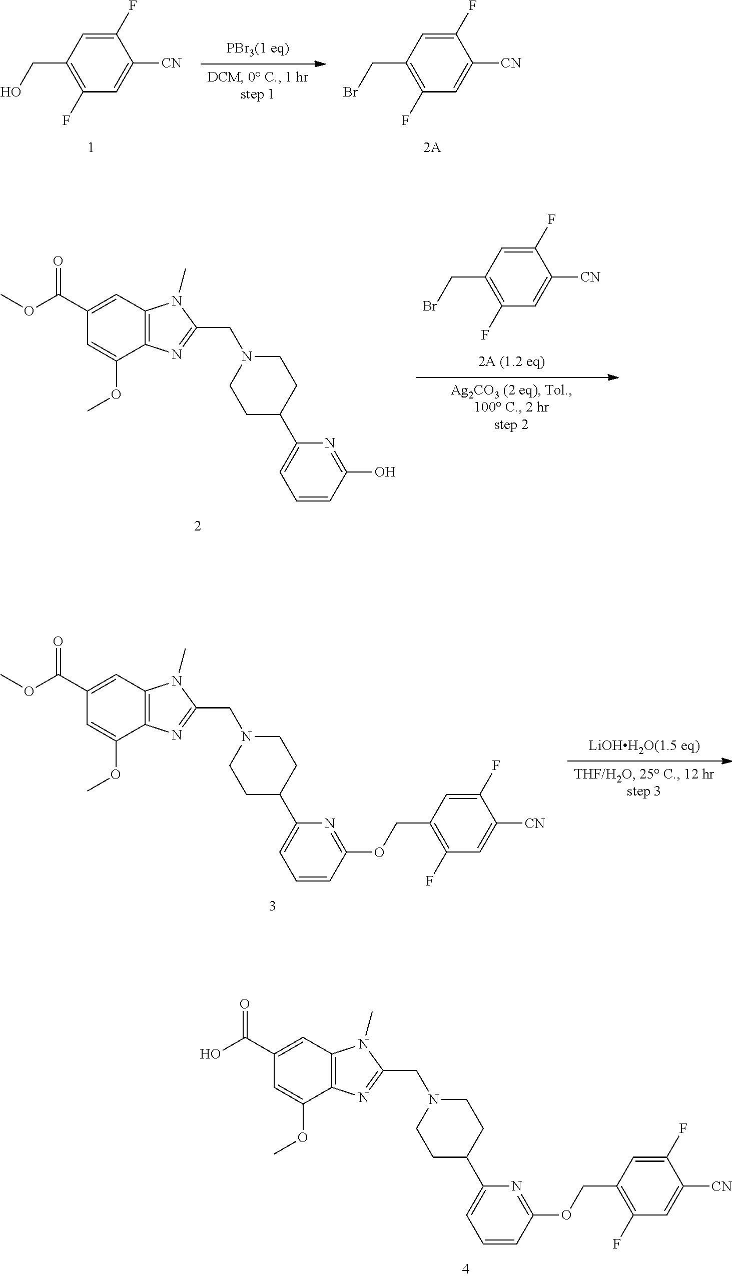

### General Procedure for Preparation of Intermediate 2A:

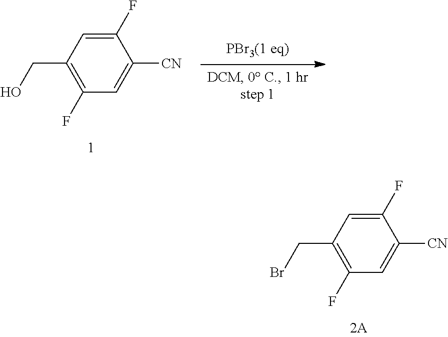 [SMILES: N#Cc1cc(F)c(CO)cc1F ; "DCM, 0° C., 1 hrstep 1" ; "PBr3(1 eq)" ; N#Cc1cc(F)c(CBr)cc1F]

Equip a 10 mL one-necked round bottom flask, addition funnel N₂ balloon. DCM (2 mL) was charged to the one-necked round bottom flask, then 1 (200 mg, 1.18 mmol, 1 eq) was added. At 0° C., PBr₃ (320.10 mg, 1.18 mmol, 1 eq) was added to the reaction mixture at 0° C. After the addition, the mixture was stirred at 0° C. for 1 hr. TLC indicated 1 was consumed completely and two new spots formed (TLC-Ethyl acetate/Methanol=5/1, Product Rf=0.43). The reaction was clean according to TLC. Then, the reaction mixture was added to 5 mL NaHCO₃ at 25° C. . . . The mixture was extracted by DCM (10 mL*3). Then organic phase was combined and washed by brine (10 mL*2), dried with Na₂SO₄, filtered. The organic phase was concentrated under reduced pressure give a residue. 2A (200 mg, 861.98 μmol, 72.89% yield) was obtained as a white solid. ¹H NMR (400 MHz, CHCl3-d) δ=7.39-7.33 (m, 2H), 4.46 (s, 2H)

### General Procedure for Preparation of Intermediate 3:

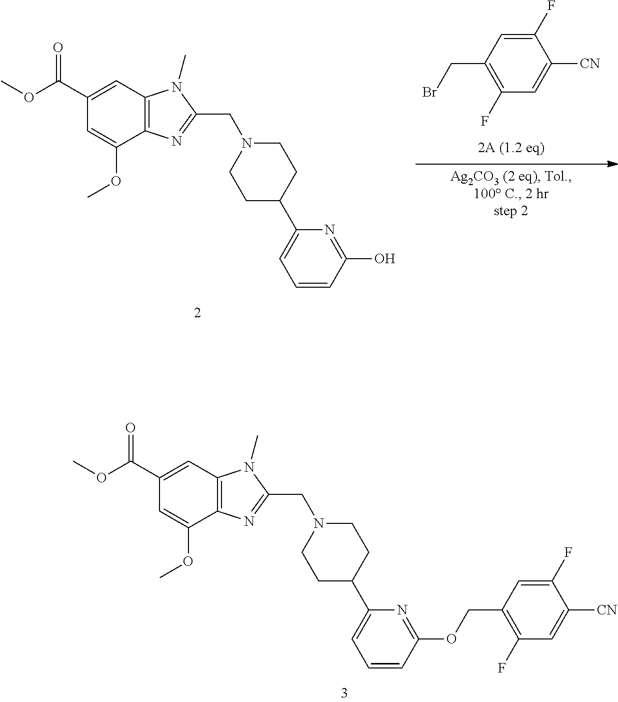 [SMILES: COC(=O)c1cc(OC)c2nc(CN3CCC(c4cccc(O)n4)CC3)n(C)c2c1 ; N#Cc1cc(F)c(CBr)cc1F ; "Ag2CO3 (2 eq), Tol.,100° C., 2 hrstep 2" ; COC(=O)c1cc(OC)c2nc(CN3CCC(c4cccc(OCc5cc(F)c(C#N)cc5F)n4)CC3)n(C)c2c1]

To a solution of 2 (200 mg, 487.25 μmol, 1 eq) and 2A (136.82 mg, 589.66 μmol, 1.2 eq) in Toluene (2 mL) was added Ag₂CO₃ (268.71 mg, 974.50 μmol, 44.22 μL, 2 eq). The mixture was stirred at 100° C. for 2 hr. LC-MS showed 2 was consumed completely and one main peak with desired mass was detected. The mixture was filtered and washed with ethyl acetate. The aqueous phase was extracted with ethyl acetate (10 mL*3) and water (10 mL). The combined organic phase was washed with brine (5 mL), dried with anhydrous Na₂SO₄, filtered and concentrated in vacuum. The residue was purified by silica gel chromatography (column height: 250 mm, diameter: 100 mm, 100-200 mesh silica gel, Petroleum ether/Ethyl acetate=1/0, 10/1). 3 (200 mg, 245.74 μmol, 50.43% yield, 69% purity) was obtained as yellow oil.

LCMS: RT=0458 min, MS cal.: 561.5, [M+H]⁺=562.3

¹H NMR (400 MHz, DMSO-d6) δ=8.05-7.99 (m, 1H), 7.85 (s, 1H), 7.70-7.61 (m, 2H), 7.28 (s, 1H), 6.90 (d, J=7.2 Hz, 1H), 6.72 (d, J=8.0 Hz, 1H), 5.43 (s, 2H), 3.96 (s, 3H), 3.91 (s, 3H), 3.88 (s, 3H), 3.83 (s, 2H), 2.93 (br d, J=8.4 Hz, 2H), 2.26-2.16 (m, 2H), 1.75 (br d, J=2.0 Hz, 3H)

### General Procedure for Preparation of Compound 327 (4):

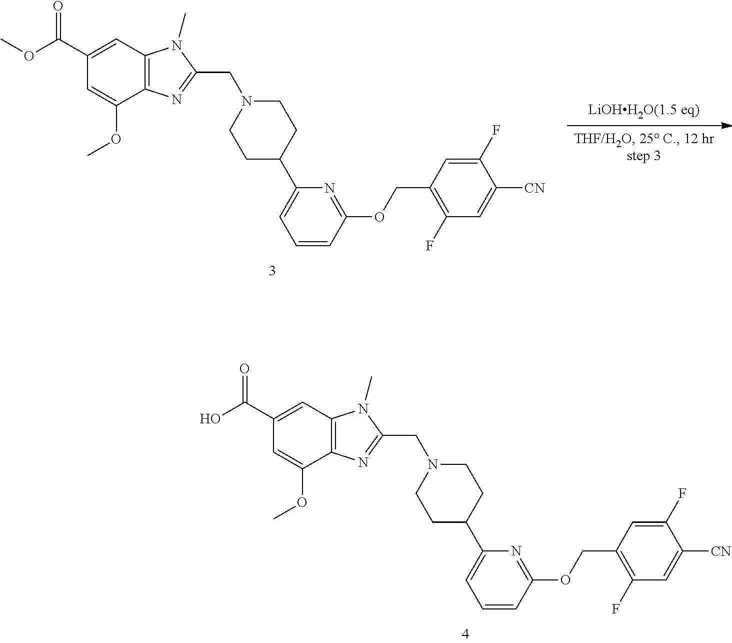 [SMILES: COC(=O)c1cc(OC)c2nc(CN3CCC(c4cccc(OCc5cc(F)c(C#N)cc5F)n4)CC3)n(C)c2c1 ; "LiOH·H2O(1.5 eq)" ; "THF/H2O, 25° C., 12 hrstep 3" ; COc1cc(C(=O)O)cc2c1nc(CN1CCC(c3cccc(OCc4cc(F)c(C#N)cc4F)n3)CC1)n2C]

To a solution of 3 (200 mg, 356.14 μmol, 1 eq) in THF (1.4 mL) was added LiOH·H₂O (22.42 mg, 534.21 μmol, 1.5 eq) in H₂O (0.6 mL). The mixture was stirred at 25° C. for 12 hr. LCMS showed 3 was consumed completely and one main peak with desired mass was detected. The mixture was concentrated under reduce pressure. The crude product was purified by prep-HPLC (column: Waters Xbridge Prep OBD C18 150*40 mm*10 um; mobile phase: [H₂O (10 mM NH₄HCO₃)-ACN]; gradient: 20%-50% B over 8.0 min). 2-((4-(6-((4-Cyano-2,5-difluorobenzyl)oxy)pyridin-2-yl)piperidin-1-yl)methyl)-4-methoxy-1-methyl-1H-benzo[d]imidazole-6-carboxylic acid (16.82 mg, 30.72 μmol, 8.63% yield) was obtained as a white solid.

HPLC: product Rt-9.460 min

LCMS: RT=1.088 min, MS cal.: 547.2, [M+H]⁺=548.2

¹H NMR (400 MHz, DMSO-d6) δ=8.02 (dd, J=5.2, 9.2 Hz, 1H), 7.75 (d, J=1.2 Hz, 1H), 7.70-7.61 (m, 2H), 7.27 (d, J=1.2 Hz, 1H), 6.89 (d, J=7.2 Hz, 1H), 6.73 (d, J=8.0 Hz, 1H), 5.42 (s, 2H), 3.94 (s, 3H), 3.89 (s, 3H), 3.79 (s, 2H), 2.91 (br d, J=11.2 Hz, 2H), 2.61-2.56 (m, 1H), 2.21-2.13 (m, 2H), 1.78-1.71 (m, 2H), 1.70-1.61 (m, 2H)

### Example A64—Preparation of Compound 328. 2-((4-(6-((4-Chloro-2-fluorobenzyl)oxy)pyridin-2-yl)piperidin-1-yl)methyl)-7-(difluoromethoxy)-3-methyl-3H-imidazo[4,5-b]pyridine-5-carboxylic acid (Compound 328) (11)

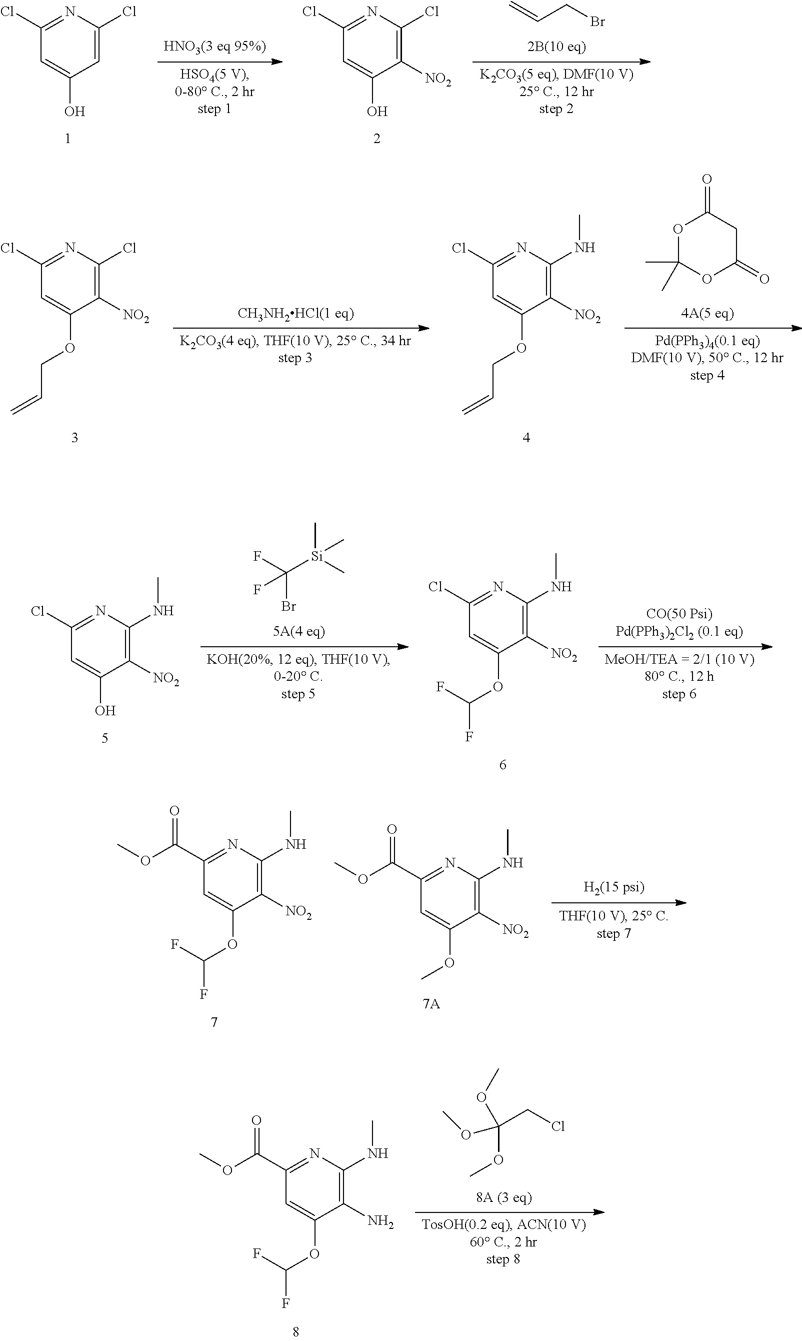

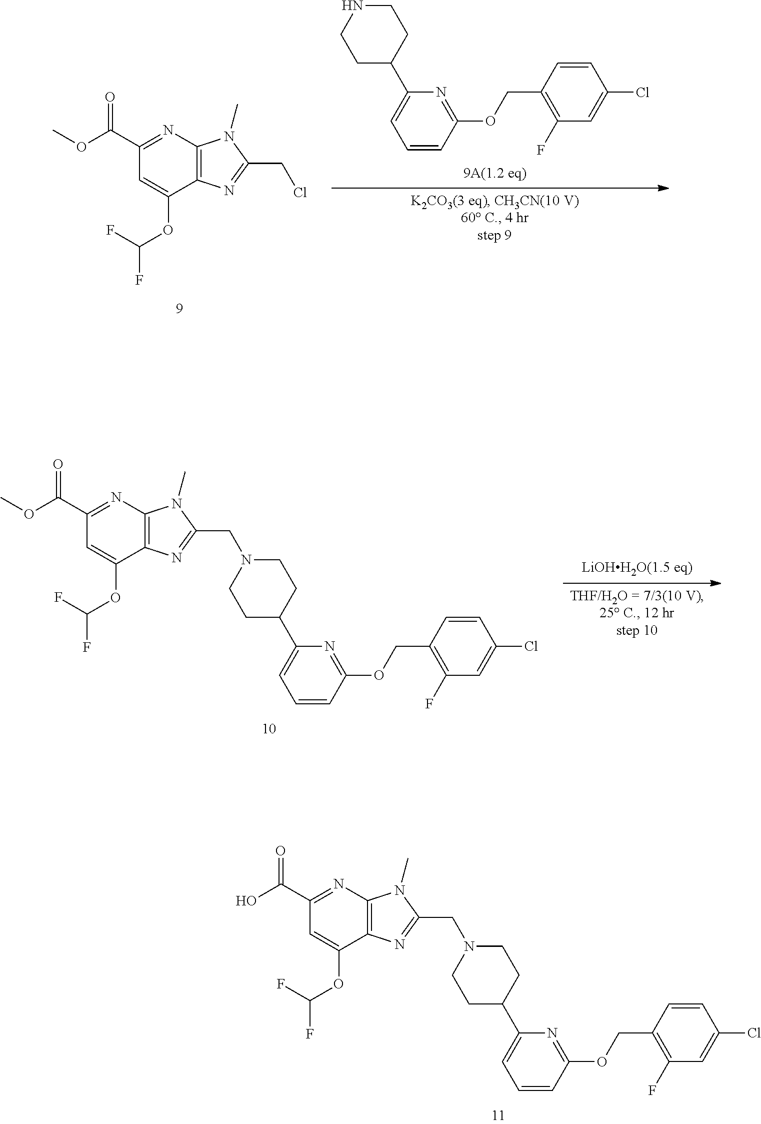

### General Procedure for Preparation of Intermediate 2

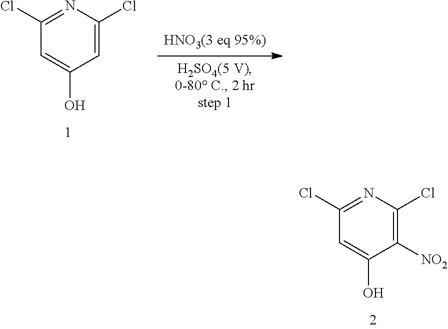 [SMILES: Oc1cc(Cl)nc(Cl)c1 ; "H2SO4(5 V),0-80° C., 2 hrstep 1" ; "HNO3(3 eq 95%)" ; O=[N+]([O-])c1c(O)cc(Cl)nc1Cl]

The reactions were carried out in parallels as 2 batches. Equip a 250 mL three-necked round bottom flask, thermometer, N₂ balloon. H₂SO₄ (50 mL) was charged to the three-necked round bottom flask, then 1 (10 g, 60.98 mmol, 1 eq) was added to the mixture at 20° C. At 0° C., HNO₃ (12.13 g, 182.94 mmol, 8.67 mL, 95% purity, 3 eq) was added in portions to the reaction mixture at 0° C. After the addition, the mixture was stirred at 80° C. for 2 hr. TLC (PE:EA=1:1) indicated 1 was consumed completely and one new spot formed. The reaction was clean according to TLC. The reaction mixture was dropwise added to ice H₂O 300 mL at 0° C. within 5 min, and then filtered. The filter cake was co-evaporated with Toluene (50 mL*3), then dried under reduced pressure to give 2 (8.2 g, 39.24 mmol, 64.34% yield) was obtained as a yellow solid.

¹H NMR (400 MHz, DMSO-d6) δ=7.16 (s, 1H)

### General Procedure for Preparation of Intermediate 3:

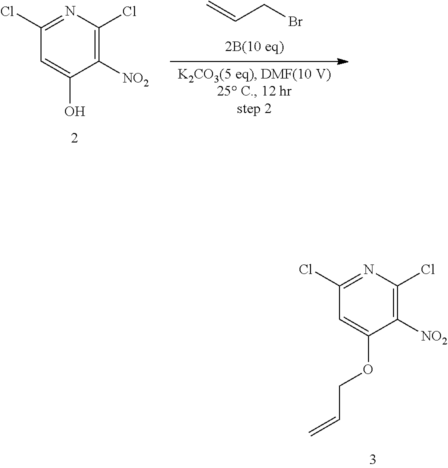

The reactions were carried out in parallels as 2 batches. To a solution of 2 (4.1 g, 19.62 mmol, 1 eq) in DMF (40 mL) was added K₂CO₃ (13.56 g, 98.09 mmol, 5 eq) and 2B (23.73 g, 196.18 mmol, 10 eq). The mixture was stirred at 25° C. for 12 hr. LC-MS showed 2 was consumed completely and one main peak with desired mass was detected. The mixture was extracted by EtOAc (100 mL*3). Then organic phase was combined and washed by H₂O (100 mL), aq. brine (100 mL), dried with Na₂SO₄, filtered. The organic phase was concentrated under reduced pressure at 45° C. to give a residue. The residue was purified by column chromatography (SiO₂, PE/EtOAc=10/1 to 0/1). 3 (7.5 g, 76.5% yield) was obtained as a yellow solid.

LCMS: Rt=1.876 min, MS cal.: 247.98/249.97, [M+H]⁺=249.0/251.0

¹H NMR (400 MHz, DMSO-d6) δ=7.75 (s, 1H), 5.96 (br d, J=10.8 Hz, 1H), 5.42-5.30 (m, 2H), 4.93 (d, J=5.2 Hz, 2H)

### General Procedure for Preparation of Intermediate 4:

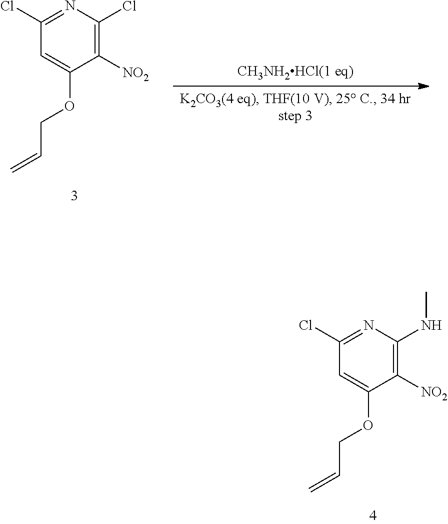 [SMILES: C=CCOc1cc(Cl)nc(Cl)c1[N+](=O)[O-] ; "CH3NH2·HCl(1 eq)" ; "K2CO3(4 eq), THF(10 V), 25° C., 34 hrstep 3" ; C=CCOc1cc(Cl)nc(NC)c1[N+](=O)[O-]]

To a solution of 3 (3.5 g, 14.05 mmol, 1 eq) in THF (35 mL) was added CH₃NH₂·HCl (474.43 mg, 7.03 mmol, 0.5 eq) and K₂CO₃ (5.83 g, 42.16 mmol, 3 eq). The mixture was stirred at 25° C. for 26 hr. After monitoring, the reaction was not completely, then was added CH₃NH₂·HCl (284.66 mg, 4.22 mmol, 0.3 eq), The mixture was stirred at 25° C. for 2 hr. After monitoring, the reaction was not completely, then was added CH₃NH₂·HCl (189.77 mg, 2.81 mmol, 0.2 eq) and K₂CO₃ (1.94 g, 14.05 mmol, 1 eq). The mixture was stirred at 25° C. for 6 hr. TLC (PE:EA=3:1) indicated 3 was consumed completely and one new spot formed. The reaction was clean according to TLC. The reaction mixture was added FA (1 M) to pH=5 at 25° C., then the mixture was diluted with H₂O (100 mL), and then extracted with EtOAc (100 mL*3). The combined organic layers were washed with brine (100 mL*2), dried over Na₂SO₄, filtered and concentrated under reduced pressure to give a residue. The residue was purified by column chromatography (SiO₂, PE/EtOAc=10/1 to 0/1). The residue was purified by prep-HPLC (column: Agela DuraShell C18 250*70 mm*10 um; mobile phase: [H₂O (10 mM NH₄HCO₃)-ACN]; gradient: 38%-63% B over 17.0 min). 4 (1.3 g, 5.34 mmol, 37.97% yield) was obtained as a yellow solid.

LCMS: Rt=1.801 min, MS cal.: 243.04/245.04, [M+H]⁺=244.0/246.0

¹H NMR (400 MHz, CHCl3-d) δ=7.78 (br s, 1H), 6.25 (s, 1H), 6.06-5.97 (m, 1H), 5.54 (dd, J=1.2, 17.2 Hz, 1H), 5.39 (dd, J=1.2, 10.6 Hz, 1H), 4.68 (td, J=1.2, 4.9 Hz, 2H), 3.10 (d, J=4.8 Hz, 3H)

### General Procedure for Preparation of Intermediate 4:

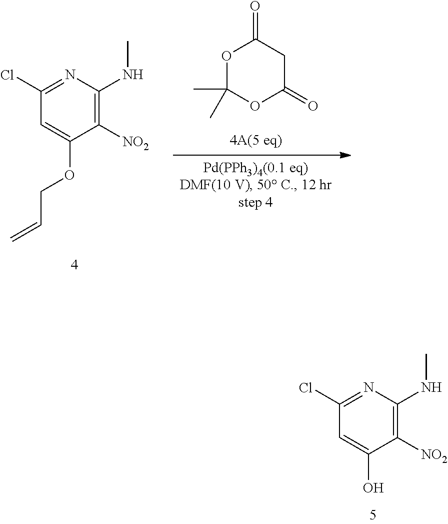

To a solution of 4 (2.75 g, 11.29 mmol, 1 eq) in DMF (30 mL) was added Pd(PPh₃)₄ (1.30 g, 1.13 mmol, 0.1 eq) and 4A (8.13 g, 56.43 mmol, 5 eq) at 25° C. The mixture was stirred at 50° C. for 12 hr. LC-MS (Rt=1.527 min) showed 4 was consumed completely and one main peak with desired mass was detected. The mixture was diluted with H₂O (100 mL), and then extracted with EtOAc (100 mL*2). The combined organic layers were washed with brine (100 mL*2), dried over Na₂SO₄, filtered and concentrated under reduced pressure to give a residue. The residue was purified by column chromatography (SiO₂, PE/EtOAc=10/1 to 0/1). The crude product was triturated with PE (5 ml) at 25° C. for 30 min. Compound 5 (0.67 g, 3.29 mmol, 29.16% yield) was obtained as a yellow solid.

LCMS: Rt=1.556 min, MS cal.: 203.01/205.01, [M+H]⁺=204.0/206.0

¹H NMR (400 MHz, CHCl3-d) δ=12.38 (s, 1H), 8.77 (br s, 1H), 6.33 (s, 1H), 3.18 (d, J=4.8 Hz, 3H).

### General Procedure for Preparation of Intermediate 6:

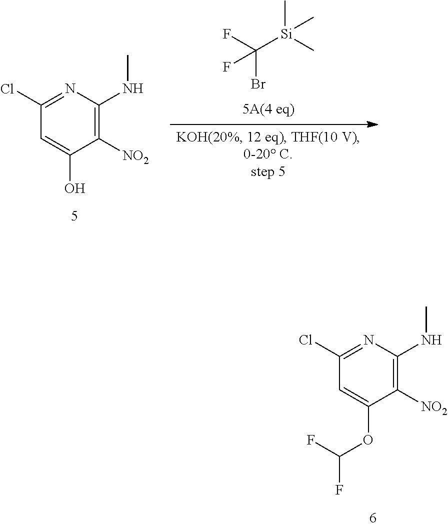

THF (4 mL) was charged to the three-necked round bottom flask, then 5 (330 mg, 1.62 mmol, 1 eq) and KOH (5.46 g, 19.45 mmol, 20% purity, 12 eq) were added at 20° C. The mixture was cooled to 0° C. At 0° C., 5A (1.32 g, 6.48 mmol, 4 eq) in THF (2 mL) was added dropwise to the reaction mixture at 0° C. After the addition, the mixture was stirred at 20° C. for 1 hr. LC-MS (Rt=0.506 min) showed 5 was consumed completely and one main peak with desired mass was detected. The reaction mixture was diluted with H₂O (100 mL) and extracted with MTBE (100 mL*3). The combined organic layers were washed with brine (100 mL), dried over Na₂SO₄, filtered and concentrated under reduced pressure to give 6 (0.77 g, 3.04 mmol, 93.66% yield) was obtained as a yellow solid.

LCMS: Rt=0.506 min, MS cal.: 253.01/255.00, [M+H]⁺=254.0/256.0

¹H NMR (400 MHz, CHCl3-d) δ=7.60 (br s, 1H), 6.65 (t, J=71.6 Hz, 2H), 3.13 (d, J=4.8 Hz, 3H)

### General Procedure for Preparation of Intermediate 7:

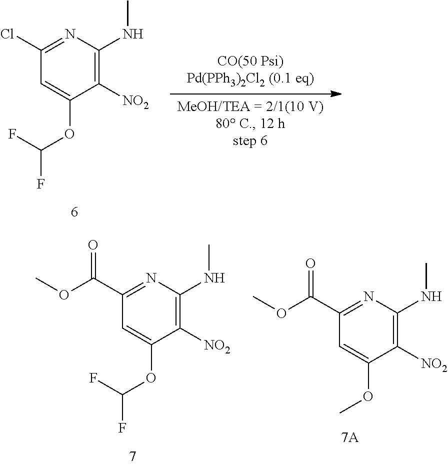

The reactions were carried out in parallels as 4 batches. Equip a 75 mL three-necked round bottom flask, addition funnel and thermometer, N₂, MeOH (2 mL) and TEA (1 mL) was charged to the three-necked round bottom flask, then 6 (200 mg, 788.67 μmol, 1 eq) was added to the mixture at 25° C. At 25° C. (inner temperature), Pd(PPh₃)₂Cl₂ (55.36 mg, 78.87 μmol, 0.1 eq) was added in portions to the reaction mixture at 25° C. After the addition, the mixture was stirred at 80° C. for 12 hr. TLC (PE:EtOAc=3:1) indicated 6 was consumed completely and two new spots formed. The reaction was messy according to TLC. The reaction mixture was filtered. The filtrate was diluted with H₂O (10 mL), the aqueous layer was extracted with EtOAc (10 mL*3). The combined organic phase was washed with brine (10 mL*2), dried with anhydrous Na₂SO₄, filtered and concentrated in vacuum. The residue was purified by column chromatography (SiO₂, PE/EtOAc=10/1 to 0/1). 7 (70 mg, 8% yield) was obtained as a yellow solid and 7A (180 mg, 746.27 μmol, 23.7% yield) was obtained as a yellow solid.

LCMS: Rt=0.450 min, MS cal.: 277.05/278.05, [M+H]⁺=278.1/278.9

¹H NMR (400 MHz, CHCl3-d) δ=7.25-7.17 (m, 1H), 6.99 (br s, 1H), 6.71 (t, J=71.4 Hz, 1H), 4.05-3.92 (m, 3H), 3.25-3.12 (m, 3H)

LCMS: Rt=0.405 min, MS cal.: 241.07/242.07, [M+H]⁺=242.0/242.9

¹H NMR (400 MHz, CHCl3-d) δ=7.19 (s, 1H), 7.01 (s, 1H), 3.95 (s, 3H), 3.90 (s, 3H), 3.09 (d, J=4.8 Hz, 3H).

### General Procedure for Preparation of Intermediate 8:

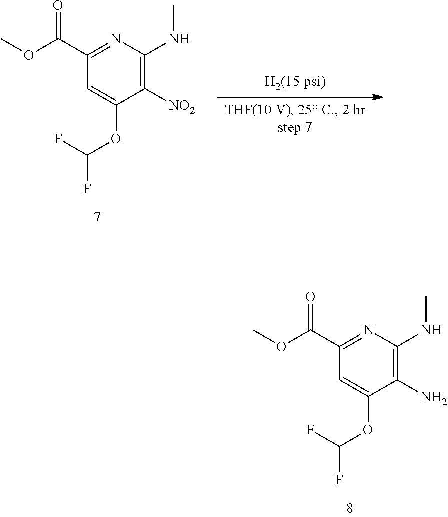

Equip a 25 mL three-necked round bottom flask, addition funnel and thermometer, H₂ balloon. THF (1 mL) was charged to the three-necked round bottom flask, then 6 (70 mg, 252.54 μmol, 1 eq) was added to the mixture at 25° C. Pd/C (80.63 mg) was added in portions to the reaction mixture at 25° C. After the addition, the mixture was stirred at 25° C. for 2 hr. LC-MS (Rt=0.298 min) showed 7 was consumed completely and one main peak with desired mass was detected. The suspension was filtered and the filter cake was washed with THF (10 mL×3). The combined filtrates were concentrated to dryness to give product. 8 (40 mg, 161.81 μmol, 64.07% yield) was obtained as a white solid.

LCMS: Rt=0.297 min, MS cal.: 247.08/248.08, [M+H]⁺=248.0/248.9

¹H NMR (400 MHz, CHCl3-d) δ=7.38 (s, 1H), 6.99 (s, 1H), 6.63 (t, J=73.2 Hz, 1H), 4.01-3.93 (m, 3H), 3.72 (br s, 2H), 3.17-3.07 (m, 3H)

### General Procedure for Preparation of Intermediate 9:

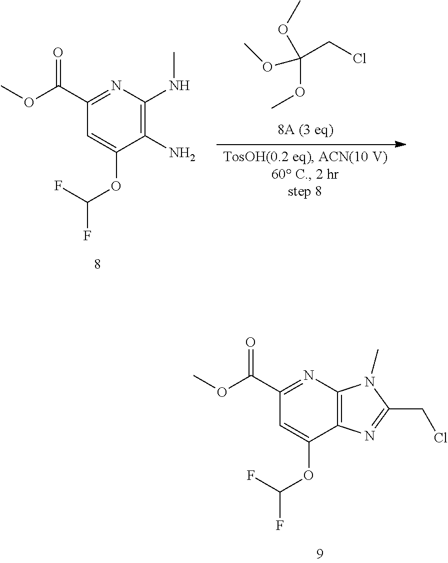

A mixture of 8 (40 mg, 161.81 μmol, 1 eq), 8A (75.04 mg, 485.44 μmol, 65.43 μL, 3 eq), TosOH (5.57 mg, 32.36 μmol, 0.2 eq) in ACN (1 mL) was degassed and purged with N₂ for 3 times, and then the mixture was stirred at 60° C. for 2 hr under N₂ atmosphere. LC-MS (Rt=0.432 min) showed 7 was consumed completely and one main peak with desired mass was detected. The reaction mixture was concentrated under reduced pressure to remove solvent. 9 (70 mg, crude) was obtained as a white solid.

LCMS: Rt=1.041 min, MS cal.: 305.04/307.03, [M+H]⁺=306.0/308.0

### General Procedure for Preparation of Intermediate 10:

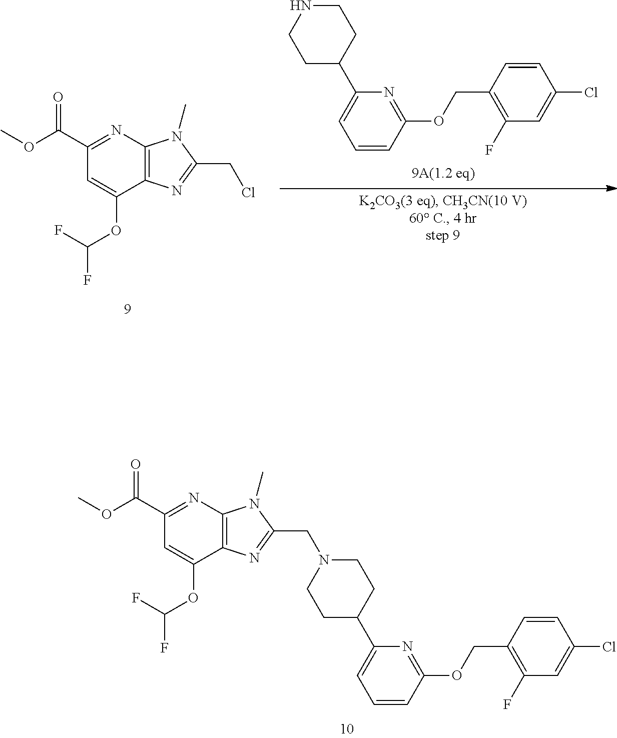 [SMILES: COC(=O)c1cc(OC(F)F)c2nc(CCl)n(C)c2n1 ; Fc1cc(Cl)ccc1COc1cccc(C2CCNCC2)n1 ; "9A(1.2 eq)" ; "K2CO3(3 eq), CH3CN(10 V)60° C., 4 hrstep 9" ; COC(=O)c1cc(OC(F)F)c2nc(CN3CCC(c4cccc(OCc5ccc(Cl)cc5F)n4)CC3)n(C)c2n1]

To a solution of 9 (70 mg, 229.01 μmol, 1 eq) in ACN (1 mL) was added K₂CO₃ (94.95 mg, 687.03 μmol, 3 eq) and 9A (88.16 mg, 274.81 μmol, 1.2 eq). The mixture was stirred at 60° C. for 2 hr. LC-MS (Rt=0.470 min) showed 9 was consumed completely and one main peak with desired mass was detected. The mixture was extracted by EtOAc (10 mL*3). Then organic phase was combined and washed by H₂O (10 mL), aq. brine (10 mL), dried with Na₂SO₄, filtered. The organic phase was concentrated under reduced pressure at 45° C. to give a residue. The residue was purified by column chromatography (SiO₂, PE/EtOAc=10/1 to 0/1). 10 (35 mg, 59.32 μmol, 25.90% yield) was obtained as a white solid.

LCMS: Rt=0.503 min, MS cal.: 589.17/591.17, [M+H]⁺=590.2/592.2

¹H NMR (400 MHz, CHCl3-d) δ=7.82 (s, 1H), 7.86 (t, J=72.0 Hz, 1H), 7.51 (t, J=8.0 Hz, 1H), 7.44 (t, J=8.0 Hz, 1H), 7.15-7.08 (m, 2H), 6.74 (d, J=7.2 Hz, 1H), 6.61 (d, J=8.4 Hz, 1H), 5.41 (s, 2H), 4.09 (s, 3H), 4.04 (s, 3H), 3.89 (s, 2H), 2.96 (br d, J=11.2 Hz, 2H), 2.62 (br s, 1H), 2.32 (br t, J=10.8 Hz, 2H), 1.91-1.76 (m, 4H).

### General Procedure for Preparation of Compound 328 (11):

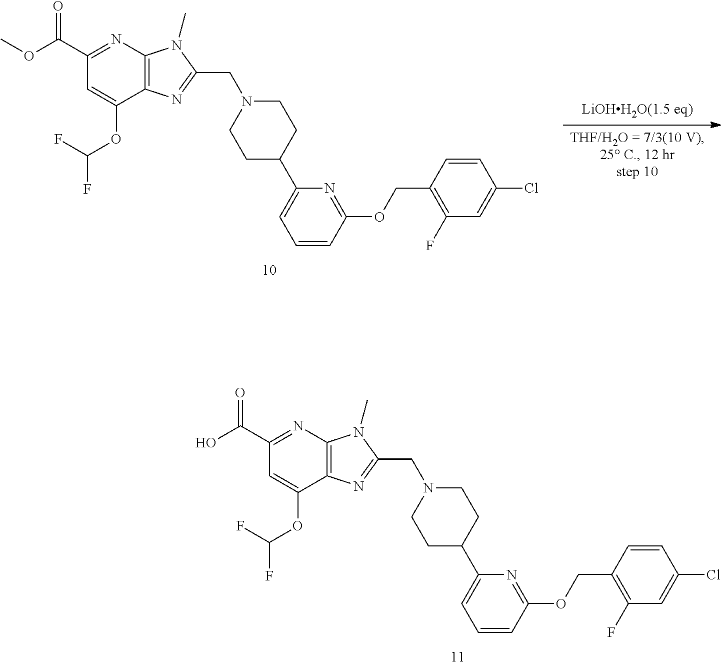

To a solution of 10 (35 mg, 59.32 μmol, 1 eq) in THF (0.7 mL) was added LiOH·H₂O (3.73 mg, 88.98 μmol, 1.5 eq) in H₂O (0.3 mL). The mixture was stirred at 25° C. for 12 hr. LC-MS (Rt=1.183 min) showed 10 was consumed completely and one main peak with desired mass was detected. The reaction was filtered directly. The residue was purified by prep-HPLC (column: Waters Xbridge BEH C18 100*30 mm*10 um; mobile phase: [H₂O (10 mM NH₄HCO₃)-ACN]; gradient: 15%-70% B over 8.0 min). 2-((4-(6-((4-Chloro-2-fluorobenzyl)oxy)pyridin-2-yl)piperidin-1-yl)methyl)-7-(difluoromethoxy)-3-methyl-3H-imidazo[4,5-b]pyridine-5-carboxylic acid (15.61 mg, 27.10 μmol, 45.69% yield) was obtained as a white solid.

HPLC: Rt=12.161 min, purity: 100.00%

LCMS: Rt=2.816 min, MS cal.: 575.15/577.15, [M+H]⁺=576.2/578.2

¹HNMR (400 MHz, DMSO-d6) δ=7.96 (t, J=73.2 Hz, 1H), 7.66 (s, 1H), 7.62 (t, J=7.8 Hz, 1H), 7.55 (t, J=8.2 Hz, 1H), 7.47-7.42 (m, 1H), 7.27 (s, 1H), 7.28 (d, J=8.0 Hz, 1H), 6.87 (d, J=7.2 Hz, 1H), 6.67 (d, J=8.0 Hz, 1H), 5.36 (s, 2H), 3.95 (s, 3H), 3.88 (s, 2H), 2.94 (br d, J=11.2 Hz, 2H), 2.61-2.52 (m, 2H), 2.32-2.15 (m, 2H), 1.84-1.65 (m, 4H)

### Example A65—Preparation of Compound 329 and Compound 330. (S)-2-((4-(6-((4-chloro-2-fluorobenzyl)oxy)pyridin-2-yl)piperidin-1-yl)methyl)-4-(1,2-difluoroethoxy)-1-methyl-1H-benzo[d]imidazole-6-carboxylic acid (Compound 329) & (R)-2-((4-(6-((4-chloro-2-fluorobenzyl)oxy)pyridin-2-yl)piperidin-1-yl)methyl)-4-(1,2-difluoroethoxy)-1-methyl-1H-benzo[d]imidazole-6-carboxylic acid (Compound 330)

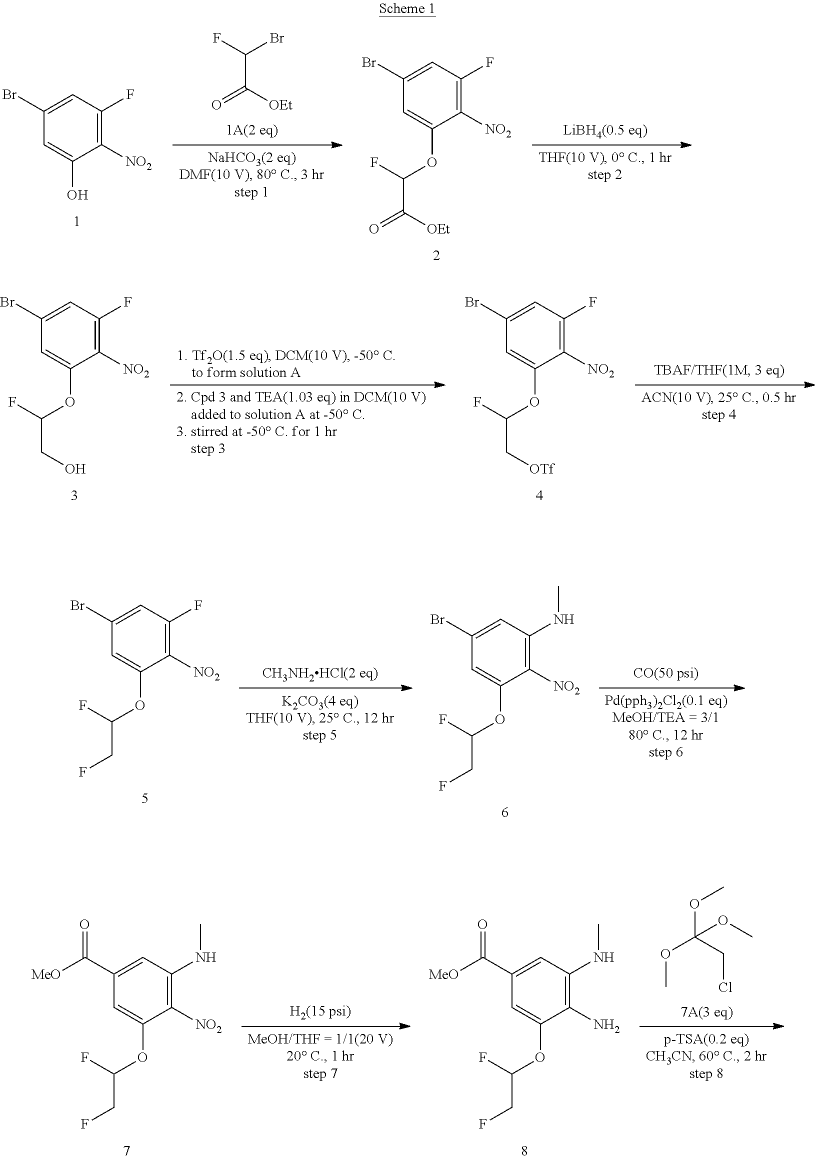

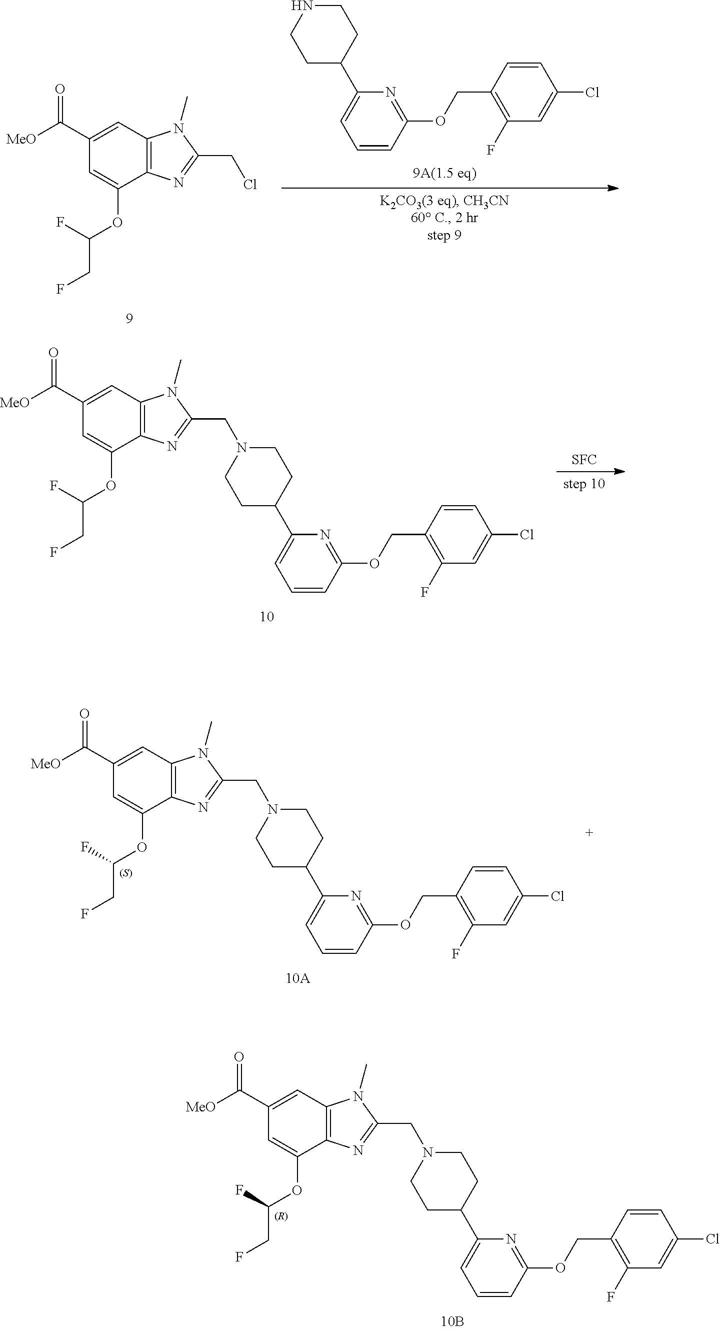

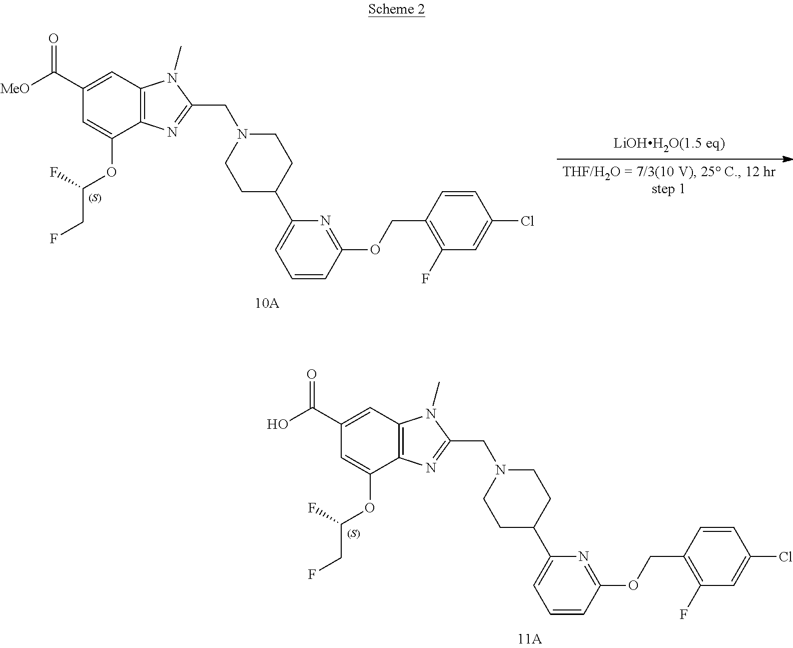

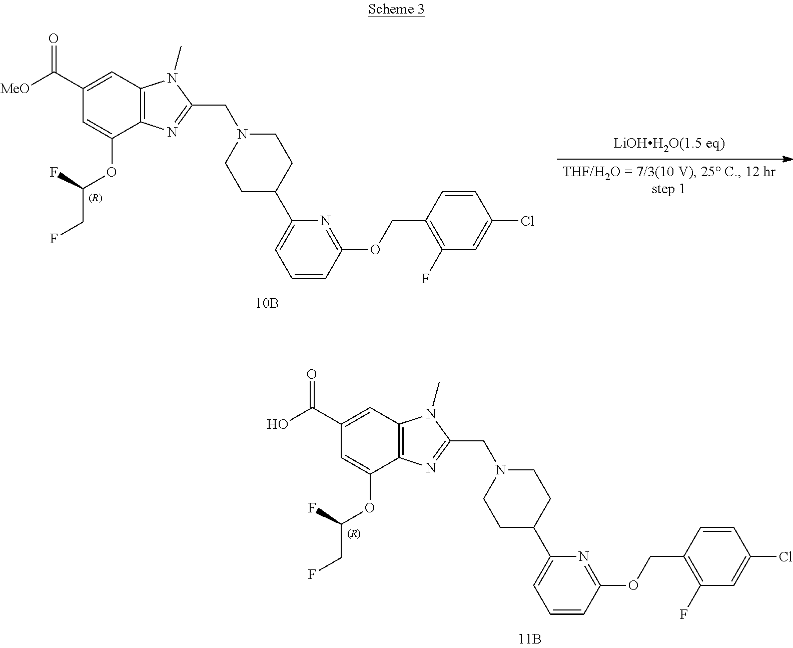

### General Procedure for Preparation of Scheme 1, Intermediate 2:

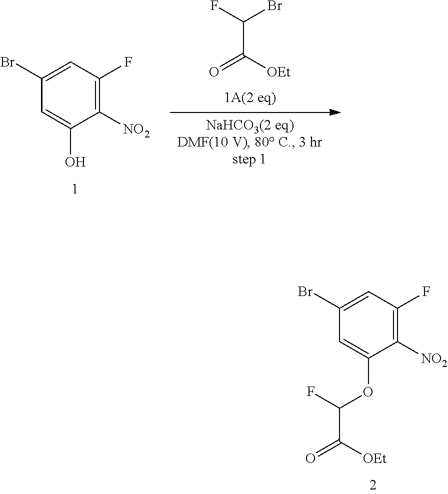

To a solution of 1 (5 g, 21.19 mmol, 1 eq) in DMF (50 mL) was added 1A (7.84 g, 42.37 mmol, 5.01 mL, 2 eq) and NaHCO₃ (3.56 g, 42.37 mmol, 2 eq). The mixture was stirred at 80° C. for 3 hr. TLC (Petroleum ether/Ethyl acetate=5/1, product R_{f}=0.46) indicated 1 was consumed almost and one new spot formed. The reaction was clean according to TLC. The reaction mixture was diluted with H₂O 30 mL and extracted with MTBE 90 mL (30 mL*3). The combined organic layers were washed with brine 30 mL, dried over Na₂SO₄, filtered, and concentrated under reduced pressure to give a residue. The residue was purified by column chromatography (SiO₂, Petroleum ether/Ethyl acetate=50/1 to 10/1). 2 (7.13 g, 20.97 mmol, 99% yield) was obtained as a yellow oil.

¹H NMR (400 MHz, CHCl3-d) δ=7.35-7.28 (m, 2H), 5.96 (d, J=57.2 Hz, 1H), 4.43-4.30 (m, 2H), 1.36 (t, J=7.2 Hz, 3H)

### General Procedure for Preparation of Scheme 1, Intermediate 3:

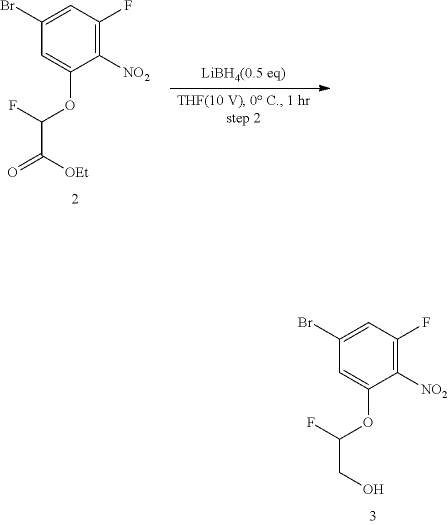 [SMILES: CCOC(=O)C(F)Oc1cc(Br)cc(F)c1[N+](=O)[O-] ; "LiBH4(0.5 eq)" ; "THF(10 V), 0° C., 1 hrstep 2" ; O=[N+]([O-])c1c(F)cc(Br)cc1OC(F)CO]

Equip a 25 mL three-necked round bottom flask, thermometer and N₂ balloon. THF (2 mL) was charged to the 25 mL three-necked round bottom flask, then 2 (200 mg, 588.11 μmol, 1 eq) was added at 25° C. At 0° C. (inner temperature), LiBH₄ (2 M, 147.03 μL, 0.5 eq) was added in portions to the reaction mixture at 0° C. After the addition, the mixture was stirred at 0° C. for 1 hr. The reaction was monitored by TLC. TLC (Petroleum ether/Ethyl acetate=3/1, product R_{f}=0.40) indicated 2 was consumed completely and one new spot formed. 5 reactions were combined for workup. The reaction mixture was added to H₂O 20 mL at 25° C. The mixture was extracted by DCM 60 mL (20 mL*3). Then organic phase was washed brine 20 mL. The organic was dried over Na₂SO₄, filtered. The organic phase was concentrated under reduced pressure at 45° C. to give a residue. The residue was purified by column chromatography (SiO₂, Petroleum ether/Ethyl acetate=20/1 to 5/1). Compound 3 (390 mg, 1.31 mmol, 44% yield) was obtained as a yellow oil.

¹H NMR (400 MHz, CHCl3-d) δ=7.34 (d, J=1.2 Hz, 1H), 7.26 (dd, J=1.6, 8.4 Hz, H), 5.84 (dt, J=8.4, 59.6 Hz, 1H), 3.92 (dd, J=4.8, 12.0 Hz, 2H)

### General Procedure for Preparation of Scheme 1, Intermediate 4:

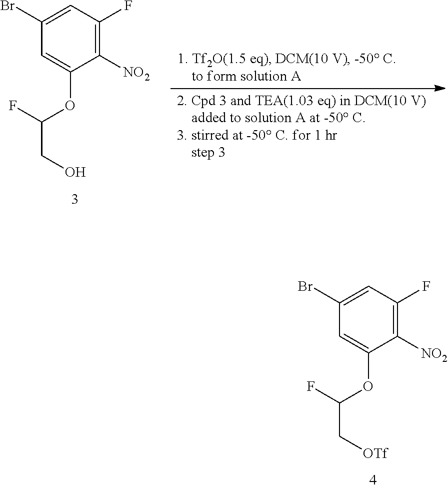

Equip a 25 mL three-necked round bottom flask, and thermometer, N₂ balloon (Solution B). DCM (4 mL) was charged to the 25 mL round bottom flask, then 3 (800 mg, 2.68 mmol, 1 eq) was added at 25° C. At −10° C. (inner temperature), TEA (279.76 mg, 2.76 mmol, 384.82 μL, 1.03 eq) was added to the reaction mixture at −10° C. to form solution B. Equip a 50 mL three-necked round bottom flask, and thermometer, N₂ balloon (Solution A). DCM (4 mL) was charged to the 50 mL round bottom flask, then Tf₂O (1.14 g, 4.03 mmol, 664.32 μL, 1.5 eq) was added at 25° C. to form solution A. Solution A was cooled to −50° C., then solution B was added to the solution A at −50° C. After the addition, the mixture was stirred at −50° C. for 1 hr. The reaction was monitored by TLC. TLC (Petroleum ether/Ethyl acetate=5/1, product R_{f}=0.45) indicated 3 was consumed completely and one new spot formed. The reaction was clean according to TLC. The reaction mixture was added to H₂O 20 mL at 25° C. The mixture was extracted by DCM 60 mL (20 mL*3). Then organic phase was washed brine 20 mL, dried over Na₂SO₄, filtered. The organic phase was concentrated under reduced pressure at 45° C. to give a residue. The residue was purified by column chromatography (SiO₂, Petroleum ether/Ethyl acetate=50/1 to 10/1). 4 (840 mg, 1.95 mmol, 73% yield) was obtained as a yellow oil.

¹H NMR (400 MHz, CHCl3-d) δ=7.36-7.32 (m, 2H), 6.03 (dt, J=4.4, 59.6 Hz, 1H), 4.74-4.67 (m, 2H)

### General Procedure for Preparation of Scheme 1, Intermediate 5:

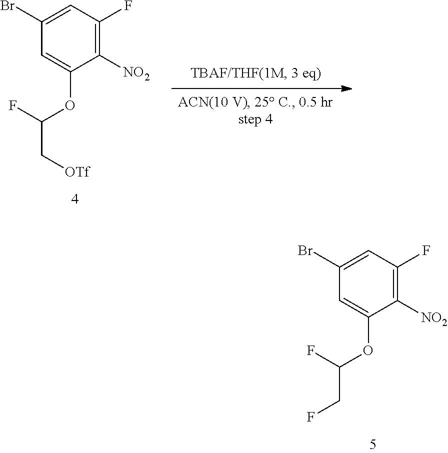 [SMILES: O=[N+]([O-])c1c(F)cc(Br)cc1OC(F)COS(=O)(=O)C(F)(F)F ; "ACN(10 V), 25° C., 0.5 hrstep 4" ; "TBAF/THF(1M, 3 eq)" ; O=[N+]([O-])c1c(F)cc(Br)cc1OC(F)CF]

To a solution of 4 (840 mg, 1.95 mmol, 1 eq) in ACN (8 mL) was added TBAF (1 M, 5.86 mL, 3 eq). The mixture was stirred at 25° C. for 0.5 hr. TLC (Petroleum ether/Ethyl acetate=7/1, product R_{f}=0.45) indicated 4 was consumed completely and one new spot formed. The reaction was clean according to TLC. The reaction mixture was partitioned between H₂O 20 mL and DCM 60 mL. The organic phase was separated, washed with brine 20 mL, dried over Na₂SO₄, filtered, and concentrated under reduced pressure to give a residue. The residue was purified by column chromatography (SiO₂, Petroleum ether/Ethyl acetate=30/1 to 10/1). 5 (500 mg, 1.67 mmol, 85% yield) was obtained as a colorless oil.

¹H NMR (400 MHz, CHCl3-d) δ=7.34 (d, J=1.2 Hz, 1H), 7.29 (dd, J=1.6, 8.4 Hz, 1H), 6.09-5.87 (m, 1H), 4.72-4.66 (m, 1H), 4.61-4.54 (m, 1H)

### General Procedure for Preparation of Scheme 1, Intermediate 6:

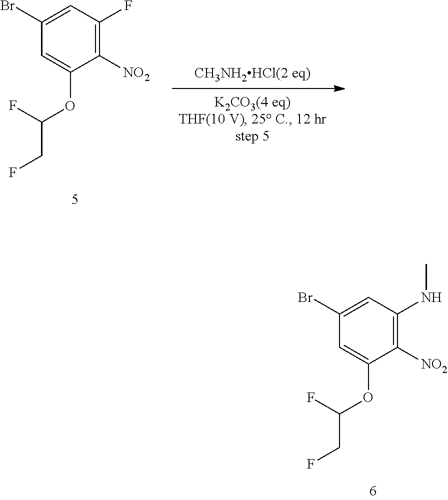 [SMILES: O=[N+]([O-])c1c(F)cc(Br)cc1OC(F)CF ; "CH3NH2·HCl(2 eq)" ; "K2CO3(4 eq)THF(10 V), 25° C., 12 hrstep 5" ; CNc1cc(Br)cc(OC(F)CF)c1[N+](=O)[O-]]

To a solution of 5 (400 mg, 1.33 mmol, 1 eq) in THF (4 mL) was added CH₃NH₂·HCl (180.03 mg, 2.67 mmol, 2 eq) and K₂CO₃ (737.02 mg, 5.33 mmol, 4 eq). The mixture was stirred at 25° C. for 12 hr. TLC (Petroleum ether/Ethyl acetate=5/1, product R_{f}=0.43) indicated 5 was consumed completely and one new spot formed. The reaction was clean according to TLC. The reaction mixture was diluted with H₂O 20 mL and extracted with EtOAc 60 mL (20 mL*3). The combined organic layers were washed with brine 10 mL, dried over Na₂SO₄, filtered and concentrated under reduced pressure to give a residue. The residue was purified by column chromatography (SiO₂, Petroleum ether/Ethyl acetate=30/1 to 10/1). 6 (370 mg, 1.19 mmol, 89% yield) was obtained as a yellow solid.

LCMS: RT=1.314 min, MS cal.: 309.9, 311.9, [M−H]⁺=308.9, 310.9

¹H NMR (400 MHz, CHCl3-d) δ=6.80-6.71 (m, 2H), 6.48 (s, 1H), 6.02-5.81 (m, 1H), 4.79-4.51 (m, 2H), 2.95 (s, 3H)

### General Procedure for Preparation of Scheme 1, Intermediate 7:

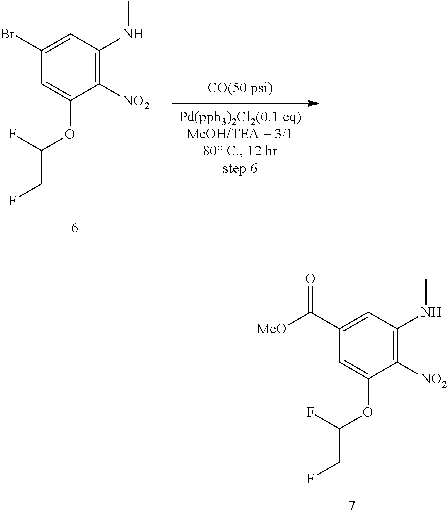

MeOH (3 mL) and TEA (1 mL) was charged to a 35 mL hydrogenated bottle, then 6 (410 mg, 1.32 mmol, 1 eq) and Pd(PPh₃)₂Cl₂ (92.51 mg, 131.80 μmol, 0.1 eq) was added. After the addition, the mixture was stirred at 80° C. for 12 hr under CO atmosphere (50Psi). The reaction was monitored by TLC. TLC (Petroleum ether/Ethyl acetate=5/1, product R_{f}=0.23) indicated 6 was consumed completely and one new spot formed. The reaction mixture was added to H₂O (10 mL). The mixture was extracted by EtOAc 30 mL (10 mL*3). Then organic phase was washed by H₂O 10 mL, brine 10 mL. The organic were dried over Na₂SO₄, filtered. The organic phase was concentrated under reduced pressure at 45° C. to give a residue. The residue was purified by prep-TLC (SiO₂, Petroleum ether/Ethyl acetate=5/1). 7 (260 mg, 895.87 μmol, 68% yield) was obtained as an orange solid.

LCMS: RT=1.221 min, MS cal.: 290.1, 291.1, [M−H]⁺=289.0, 290.0

¹H NMR (400 MHz, CHCl3-d) δ=7.29 (d, J=1.2 Hz, 1H), 7.20 (s, 1H), 6.06-5.89 (m, 1H), 4.80-4.53 (m, 2H), 3.96 (s, 3H), 3.00 (s, 3H)

### General Procedure for Preparation of Scheme 1, Intermediate 8:

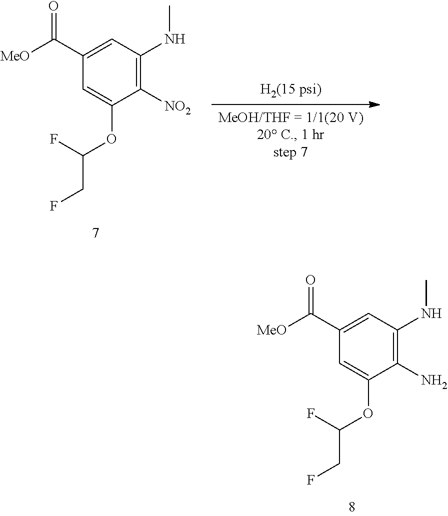

Equip a 50 mL round bottom flask, H₂ balloon. Pd/C (100 mg) was charged to the 50 mL round bottom flask under Ar atmosphere, then MeOH (1.5 mL) and 7 (260 mg, 895.87 μmol, 1 eq) in THF (1.5 mL) was added to the reaction mixture. After the addition, the mixture was stirred at 20° C. for 1 hr under H₂ atmosphere (15 Psi). The reaction was monitored by TLC. TLC (Petroleum ether/Ethyl acetate=3/1, product R_{f}=0.25) indicated 7 was consumed completely and one new spot formed. The reaction was clean according to TLC. The reaction mixture was filtered through a Celite pad and the filter cake was washed with EtOAc 30 mL (10 mL*3). The filtrate was concentrated under reduced pressure at 45° C. to give the product. 8 (210 mg, 806.96 μmol, 90% yield) was obtained as a brown solid.

LCMS: RT=1.037 min, MS cal.: 260.1, [M+H]⁺=260.9

¹H NMR (400 MHz, CHCl3-d) δ=7.41 (s, 1H), 7.27-7.26 (m, 1H), 6.04-5.82 (m, 1H), 4.75-4.56 (m, 2H), 3.89 (s, 3H), 2.94 (s, 3H)

### General Procedure for Preparation of Scheme 1, Intermediate 9:

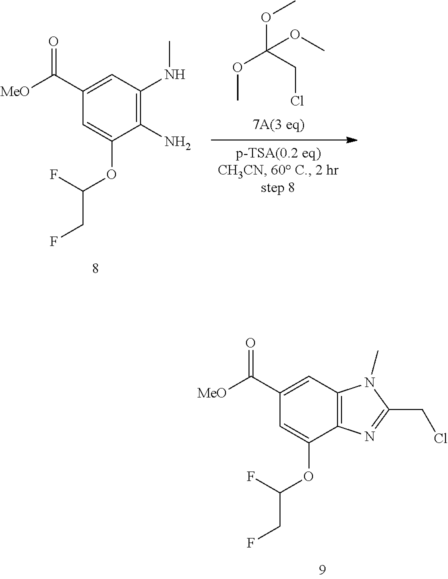

To a solution of 8 (210 mg, 806.96 μmol, 1 eq) in ACN (3 mL) was added 7A (374.25 mg, 2.42 mmol, 326.28 μL, 3 eq) and TosOH (27.79 mg, 161.39 μmol, 0.2 eq). The mixture was stirred at 60° C. for 2 hr. TLC (Petroleum ether/Ethyl acetate=1/1, product Rf=0.26) indicated 8 was consumed completely and one new spot formed. The reaction was clean according to TLC. The reaction mixture was concentrated under reduced pressure to remove solvent. The residue was diluted with H₂O 20 mL and extracted with EtOAc 60 mL (20 mL*3). The combined organic layers were washed with brine 20 mL, dried over Na₂SO₄, filtered, and concentrated under reduced pressure to give the product. 9 (240 mg, 753.05 μmol, 93% yield) was obtained as a brown solid.

LCMS: RT=1.117 min, MS cal.: 318.1, 320.1, [M+H]⁺=318.9, 320.8

¹H NMR (400 MHz, CHCl3-d) δ=7.94 (d, J=1.1 Hz, 1H), 7.72 (s, 1H), 6.68-6.46 (m, 1H), 4.88 (s, 2H), 4.87-4.67 (m, 2H), 3.98 (s, 3H), 3.94 (s, 3H)

### General Procedure for Preparation of Scheme 1, Intermediate 10:

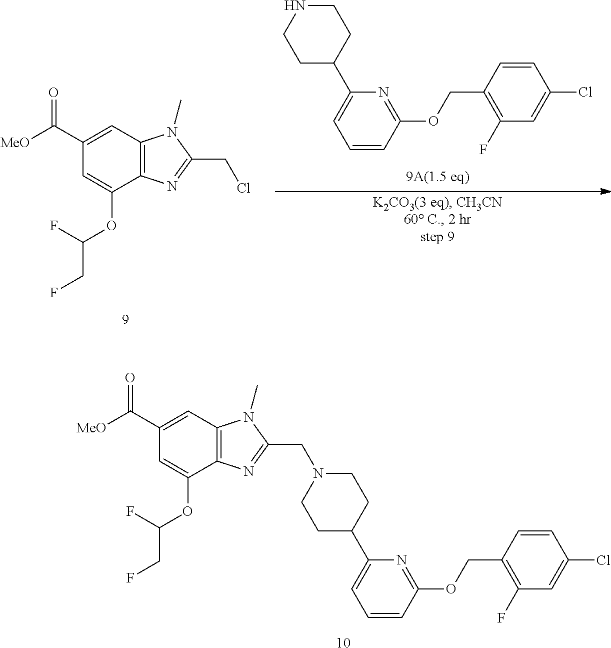

To a solution of 9 (240 mg, 753.05 μmol, 1 eq) in ACN (3 mL) was added K₂CO₃ (312.23 mg, 2.26 mmol, 3 eq) and 9A (241.57 mg, 753.05 μmol, 1 eq). The mixture was stirred at 60° C. for 2 hr. TLC (Petroleum ether/Ethyl acetate=0/1, product R_{f}=0.23) indicated 9 was consumed completely and one new spot formed. The reaction was clean according to TLC. The reaction mixture diluted with H₂O 10 mL and extracted with EtOAc 30 mL (10 mL*3). The combined organic layers were washed with brine 10 mL, dried over Na₂SO₄, filtered, and concentrated under reduced pressure to give a residue. The residue was purified by column chromatography (SiO₂, Petroleum ether/Ethyl acetate=10/1 to I/O). 10 (350 mg, 580.40 μmol, 77.07% yield) was obtained as a brown solid.

LCMS: RT=1.709 min, MS cal.: 602.2, 604.2, [M+H]⁺=603.0, 604.9

¹H NMR (400 MHz, CHCl3-d) δ=7.94 (d, J=1.2 Hz, 1H), 7.70 (s, 1H), 7.54-7.41 (m, 2H), 7.14-7.08 (m, 2H), 6.73 (d, J=7.2 Hz, 1H), 6.68-6.48 (m, 1H), 6.61 (d, J=8.0 Hz, 1H), 5.41 (s, 2H), 4.96-4.67 (m, 2H), 4.00 (s, 3H), 3.97 (s, 3H), 3.90 (s, 2H), 2.96 (s, 2H), 2.62 (t, J=11.2 Hz, 1H), 2.37-2.24 (m, 2H), 1.96-1.74 (m, 4H)

### General Procedure for Preparation of Scheme 1, Intermediate 10A& Intermediate 10B:

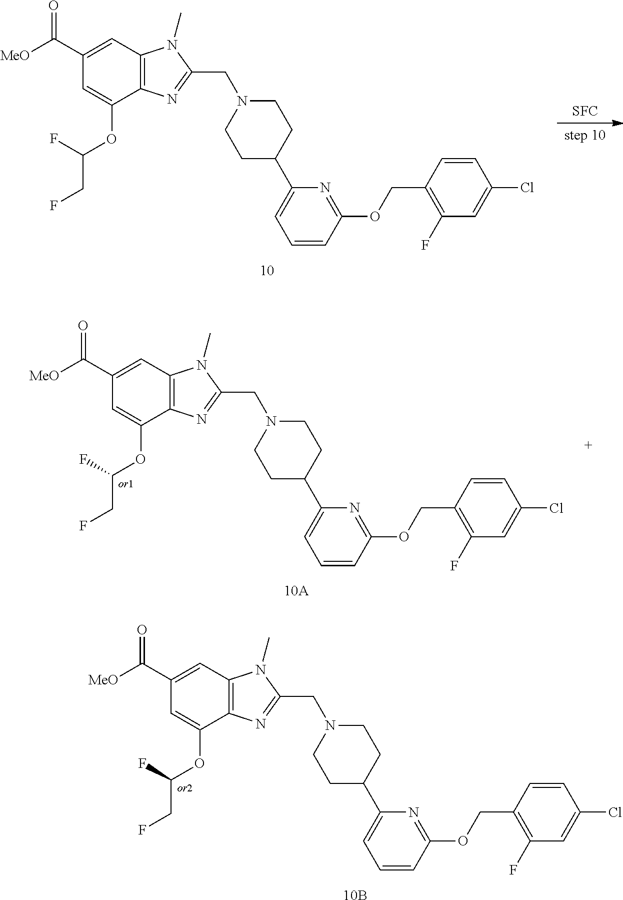

(350 mg, 580.40 μmol) was separated by SFC (condition, column: DAICEL CHIRALPAK IG (250 mm*30 mm, 10 um); mobile phase: [CO₂-MeOH (0.1% NH₃H₂O)]; B %: 60%, isocratic elution mode). 10A (170 mg, 281.91 μmol, 49% yield) was obtained as a white solid. 10B (170 mg, 281.91 μmol, 49% yield) was obtained as a white solid.

### General Procedure for Preparation of Compound 329 (11A):

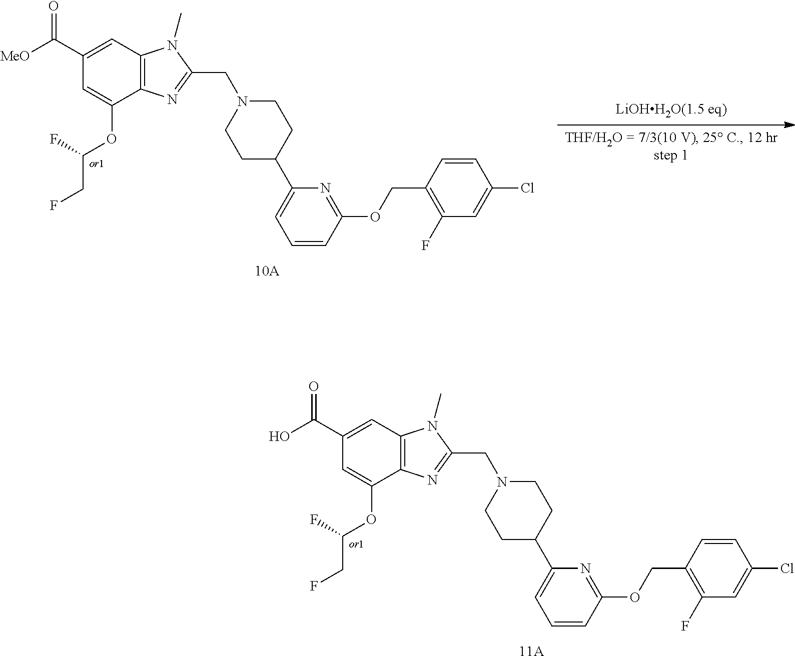

To a solution of 10A (70.00 mg, 116.08 μmol, 1 eq) in THF (0.7 mL) was added LiOH·H₂O (7.31 mg, 174.12 μmol, 1.5 eq) in H₂O (0.3 mL). The mixture was stirred at 25° C. for 1 hr. LC-MS (product RT=1.183) showed 10A was consumed completely and one main peak with desired mass was detected. The mixture was purified directly. The residue was purified by prep-HPLC (neutral condition, column: Waters Xbridge Prep OBD C18 150*40 mm*10 um; mobile phase: [H₂O (10 mM NH₄HCO₃)-ACN]; gradient: 25%-55% B over 8.0 min). (S)-2-((4-(6-((4-Chloro-2-fluorobenzyl)oxy)pyridin-2-yl)piperidin-1-yl)methyl)-4-(1,2-difluoroethoxy)-1-methyl-1H-benzo[d]imidazole-6-carboxylic acid (28.53 mg, 48.44 μmol, 41.73% yield, 100% purity) was obtained as a white solid.

LCMS: RT=2.429 min, MS cal.: 588.2, 590.2, [M+H]⁺=589.1, 591.1

HPLC: RT=12.185 min, purity: 100.00%

1H NMR (400 MHz, MeOH-d4) δ=8.03 (s, 1H), 7.71 (s, 1H), 7.57 (t, J=7.6 Hz, 1H), 7.49 (t, J=8.0 Hz, 1H), 7.23-7.14 (m, 2H), 6.82 (d, J=7.2 Hz, 1H), 6.63 (d, J=8.0 Hz, 1H), 6.56-6.34 (m, 1H), 5.40 (s, 2H), 4.86-4.64 (m, 2H), 4.02 (s, 3H), 3.96 (s, 2H), 3.07 (d, J=10.8 Hz, 2H), 2.71-2.61 (m, 1H), 2.43-2.32 (m, 2H), 1.88 (d, J=2.4 Hz, 4H)

### General Procedure for Preparation of Compound 330 (11B):

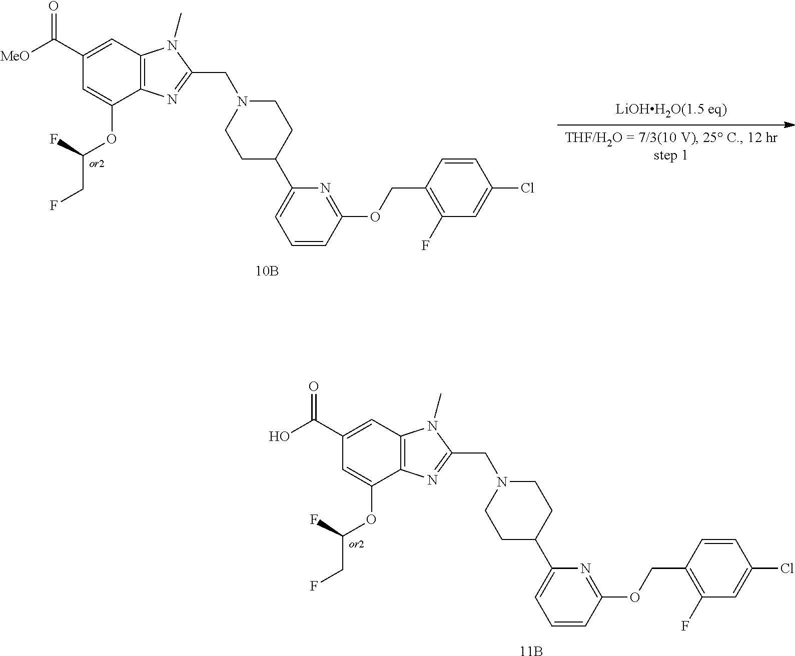

To a solution of 10B (70 mg, 116.08 μmol, 1 eq) in THF (0.7 mL) was added LiOH·H₂O (7.31 mg, 174.12 μmol, 1.5 eq) in H₂O (0.3 mL). The mixture was stirred at 25° C. for 12 hr. LCMS (RT=1.199 min) showed the 10B was consumed completely. The crude product was purified by reversed-phase HPLC (column: Waters Xbridge Prep ACN]; gradient: 30%-60% B over 8.0 min). (R)-2-((4-(6-((4-Chloro-2-fluorobenzyl)oxy)pyridin-2-yl)piperidin-1-yl)methyl)-4-(1,2-difluoroethoxy)-1-methyl-1H-benzo[d]imidazole-6-carboxylic acid (26.09 mg, 44.30 μmol, 38.16% yield) was obtained as a white solid.

LCMS: RT=2.802 min, MS cal.: 589.01, [M+H]⁺=589.2

HPLC: RT=12.185 min, purity=98.51%

SFC: RT=2.025 min, ee %=99.78%

¹H NMR (400 MHz, DMSO-d₆) δ=7.98 (d, J=1.2 Hz, 1H), 7.66-7.59 (m, 1H), 7.55 (t, J=8.4 Hz, 1H), 7.51 (s, 1H), 7.45 (dd, J=2.0, 10.0 Hz, 1H), 7.28 (dd, J=1.6, 8.0 Hz, 1H), 6.91-6.84 (m, 1H), 6.76-6.69 (m, 1H), 6.68-6.64 (m, 1H), 5.35 (s, 2H), 4.97-4.68 (m, 2H), 3.95 (s, 3H), 3.84 (d, J=1.2 Hz, 2H), 2.99-2.87 (m, 2H), 2.65-2.54 (m, 1H), 2.20 (t, J=11.6 Hz, 2H), 1.84-1.61 (m, 4H)

### Example A66—Preparation of Compound 297. 2-((4-(6-((4-Chloro-2,3-dihydrobenzofuran-7-yl)methoxy)pyridin-2-yl)piperidin-1-yl)methyl)-4-methoxy-1-methyl-1H-benzo[d]imidazole-6-carboxylic acid (Compound 297) (3)

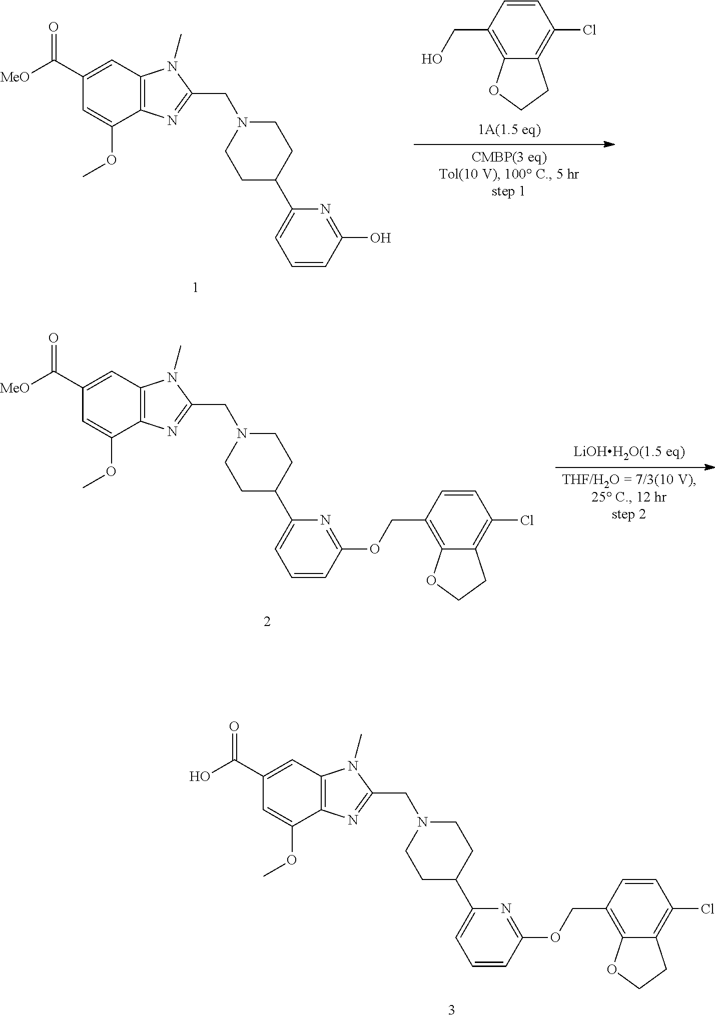

### General Procedure for Preparation of Intermediate 2:

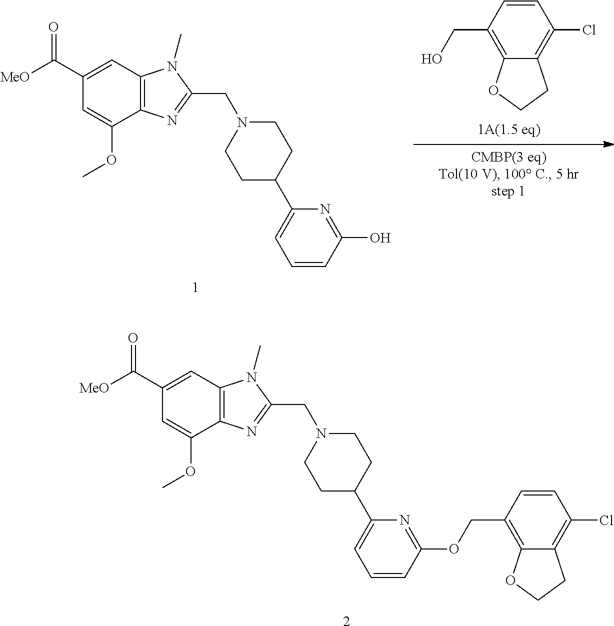

A mixture of 1 (100 mg, 243.63 μmol, 1 eq), 1A (67.47 mg, 365.44 μmol, 1.5 eq), CMBP (176.40 mg, 730.88 μmol, 3 eq) in Toluene (1 mL) was degassed and purged with N₂ for 3 times, and then the mixture was stirred at 100° C. for 5 hr under N₂ atmosphere. LCMS (product: RT=1.649 min) showed 1 was consumed completely. The reaction mixture was diluted with H₂O 10 mL and extracted with EtOAc (15 mL*3). The combined organic layers were washed with brine (10 mL*2), dried over Na₂SO₄, filtered, and concentrated under reduced pressure to give a residue. The residue was purified by column chromatography (SiO₂, Petroleum ether/Ethyl acetate=100/1 to 0/1). 2 (70 mg, 121.30 μmol, 49.79% yield) was obtained as a yellow solid.

LCMS: RT=1.642 min, MS cal.: 577.08, [M+H]⁺=577.1

¹H NMR (400 MHz, CHCl3-d) δ=7.78 (s, 1H), 7.47 (t, J=7.6 Hz, 1H), 7.39 (s, 1H), 7.21 (d, J=8.0 Hz, 1H), 6.81 (d, J=8.4 Hz, 1H), 6.71 (d, J=7.2 Hz, 1H), 6.60 (d, J=8.0 Hz, 1H), 5.31 (s, 3H), 4.66 (t, J=8.8 Hz, 2H), 4.07 (s, 3H), 4.01-3.93 (m, 5H), 3.89 (br s, 2H), 3.26 (t, J=8.8 Hz, 2H), 2.98 (br dd, J=2.0, 4.0 Hz, 2H), 2.62 (br s, 1H), 2.29 (br s, 2H), 2.00-1.71 (m, 4H)

### General Procedure for Preparation of Compound 297 (3):

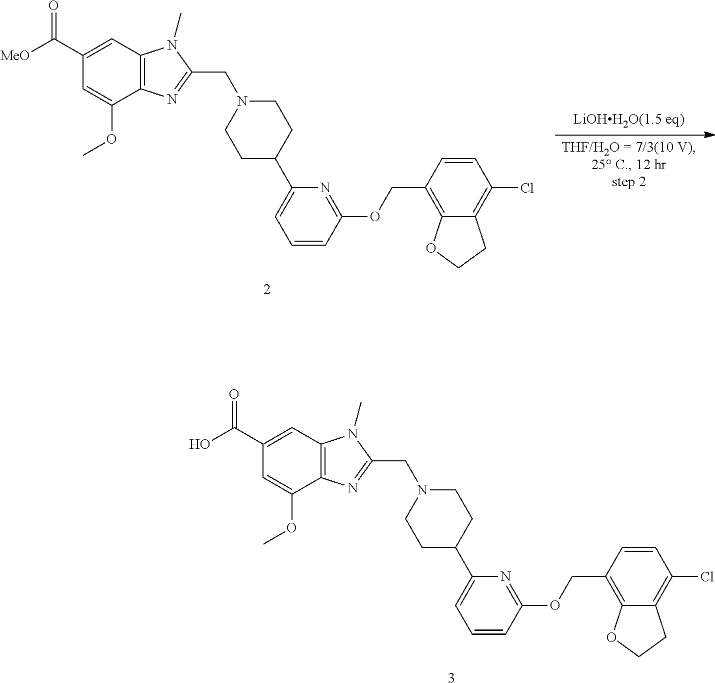

A mixture of 2 (60 mg, 103.97 μmol, 1 eq), LiOH·H₂O (6.54 mg, 155.96 μmol, 1.5 eq) in THF (0.7 mL) and H₂O (0.3 mL) was degassed and purged with N₂ for 3 times, and then the mixture was stirred at 25° C. for 12 hr under N₂ atmosphere. LCMS (product: RT=1.141 min) showed the 2 was consumed completely. The crude product was purified by reversed-phase HPLC (column: Waters Xbridge Prep OBD C18 150*40 mm*10 um; mobile phase: [H₂O (10 mM NH₄HCO₃)-ACN]; gradient: 30%-80% B over 8.0 min). Compound 297 (23.89 mg, 42.43 μmol, 40.81% yield) was obtained as a white solid.

LCMS: RT=2.673 min, MS cal.: 563.05, [M+H]⁺=563.2

HPLC: RT=11.307 min, purity=99.462%

¹H NMR (400 MHz, DMSO-d₆) δ=7.76 (s, 1H), 7.63-7.56 (m, 1H), 7.26 (s, 1H), 7.21 (d, J=8.0 Hz, 1H), 6.88-6.81 (m, 2H), 6.62 (d, J=8.0 Hz, 1H), 5.20 (s, 2H), 4.64 (t, J=8.8 Hz, 2H), 3.94 (s, 3H), 3.88 (s, 3H), 3.79 (s, 2H), 3.21 (t, J=8.8 Hz, 3H), 2.94 (br d, J=10.8 Hz, 2H), 2.63-2.53 (m, 1H), 2.23-2.12 (m, 2H), 1.84-1.65 (m, 4H)

### Example A67—Preparation of Compound 331. 2-((4-(6-((4-Chloro-2,3-dihydrobenzofuran-7-yl)methoxy)pyridin-2-yl)piperidin-1-yl)methyl)-4-(difluoromethoxy)-1-methyl-1H-benzo[d]imidazole-6-carboxylic acid (Compound 331) (7)

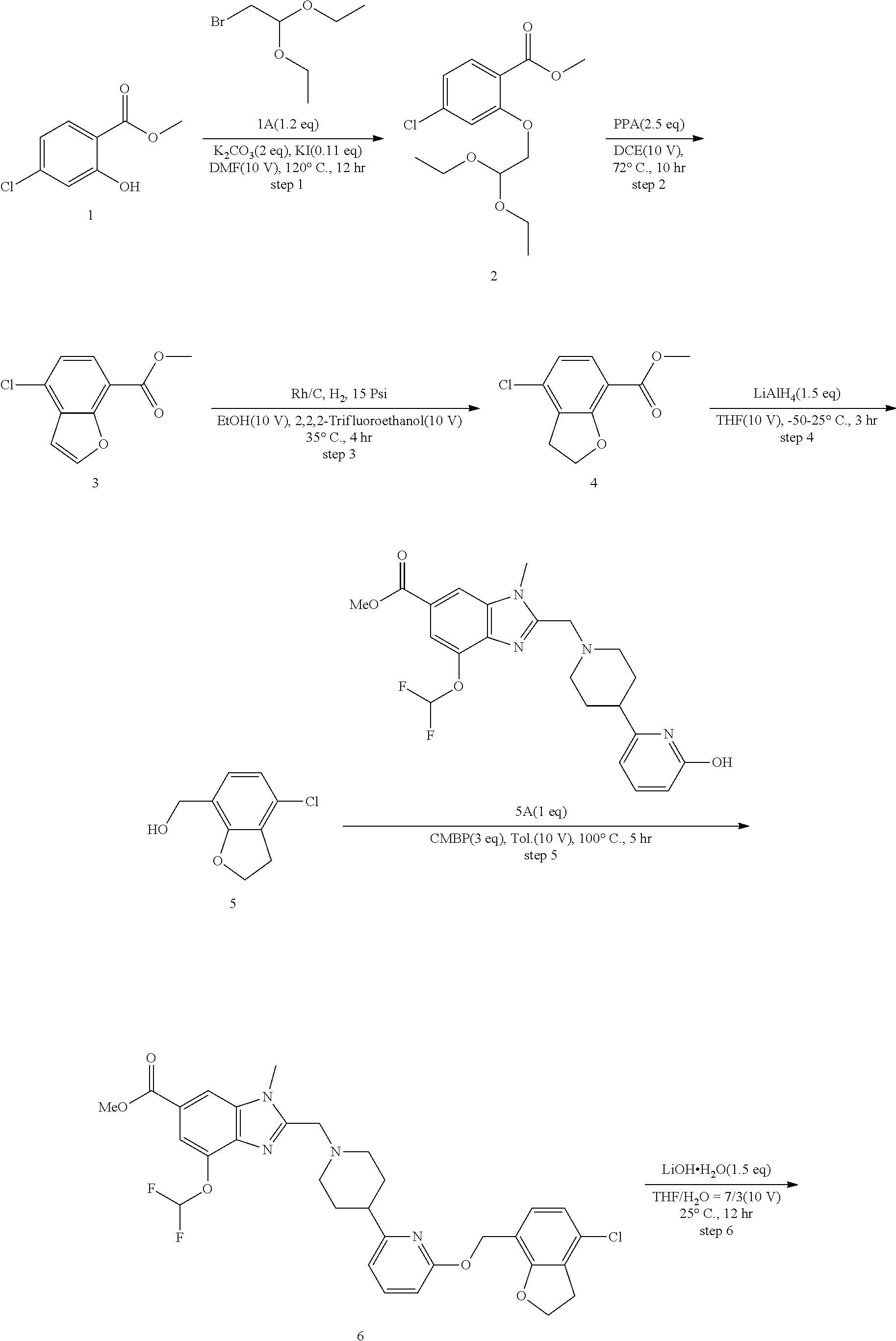

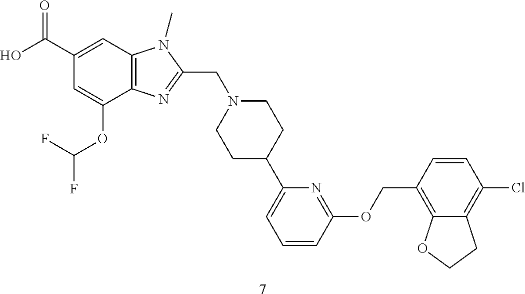 [SMILES: Cn1c(CN2CCC(c3cccc(OCc4ccc(Cl)c5c4OCC5)n3)CC2)nc2c(OC(F)F)cc(C(=O)O)cc21 ; "7"]

### General Procedure for Preparation of Intermediate 2:

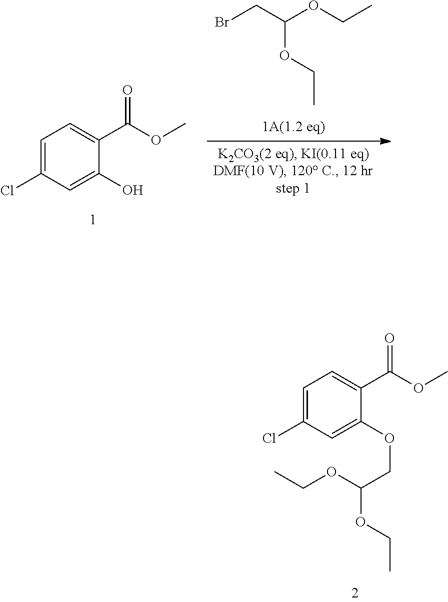

The reactions were carried out in parallels as 2 batches. To a solution of 1 (8 g, 42.87 mmol, 1 eq), 1A (10.14 g, 51.45 mmol, 7.74 mL, 1.2 eq) in DMF (80 mL) was added KI (782.90 mg, 4.72 mmol, 0.11 eq) and K₂CO₃ (11.85 g, 85.75 mmol, 2 eq). The mixture was stirred at 120° C. for 12 hr. TLC (PE:EA=5:1) indicated 1 was consumed completely and one new spot formed. The reaction was clean according to TLC. The reactions were combined to work-up. The reaction mixture was diluted with H₂O (500 mL) and extracted with EtOAc (500 mL*3). The combined organic layers were washed with brine (300 mL), dried over Na₂SO₄, filtered, and concentrated under reduced pressure to give a residue. The residue was purified by column chromatography (SiO₂, PE/EtOAc=10/1 to 0/1). 2 (11.8 g, 38.98 mmol, 45.45% yield) was obtained as a yellow oil.

¹H NMR (400 MHz, CHCl3-d) δ=7.74 (d, J=8.4 Hz, 1H), 7.02-6.95 (m, 2H), 4.86 (s, 1H), 4.05 (d, J=5.2 Hz, 2H), 3.86 (s, 3H), 3.84-3.63 (m, 4H), 1.24 (t, J=7.2 Hz, 6H)

### General Procedure for Preparation of Intermediate 2:

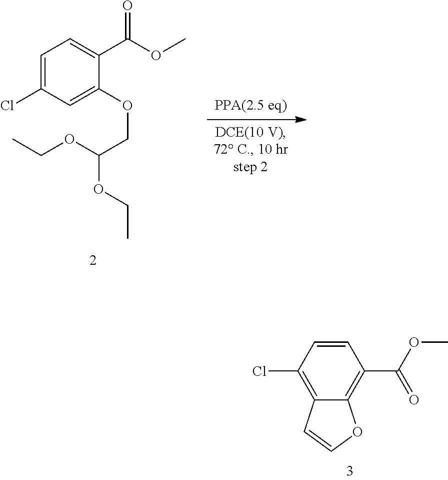 [SMILES: CCOC(COc1cc(Cl)ccc1C(=O)OC)OCC ; "DCE(10 V),72° C., 10 hrstep 2" ; "PPA(2.5 eq)" ; COC(=O)c1ccc(Cl)c2ccoc12]

To a solution of 2 (5.2 g, 17.18 mmol, 1 eq) in DCE (55 mL) was added PPA (17.07 g, 42.94 mmol, 85% purity, 2.5 eq). The mixture was stirred at 85° C. for 12 hr. TLC indicated 2 was consumed, and one major new spot with larger polarity was detected. The reaction was combined with ET77627-378 to work-up. The aqueous phase was extracted with DCM (100 mL*3). The combined organic phase was washed with brine (100 mL), dried with anhydrous Na₂SO₄, filtered and concentrated in vacuum. The residue was purified by column chromatography (SiO₂, Petroleum ether/Ethyl acetate=100/1 to 10/1). 3 (1.8 g, 8.55 mmol, 25.71% yield) was obtained as a white solid.

¹H NMR (400 MHz, CHCl3-d) δ=7.93 (d, J=8.4 Hz, 1H), 7.80 (d, J=2.4 Hz, 1H), 7.32 (d, J=8.4 Hz, 1H), 6.95 (d, J=2.4 Hz, 1H), 4.02 (s, 3H)

### General Procedure for Preparation of Intermediate 4:

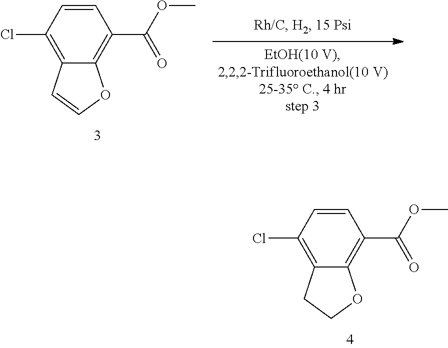

2. 2,2,2-Trifluoroethanol (9 mL) was charged to the 100 mL round bottom flask, then 3 (900 mg, 4.27 mmol, 1 eq) was added to the mixture at 25° C. Rh/C (175.90 mg) in EtOH (9 mL) was added to the reaction mixture at 25° C. under Ar atmosphere. After the addition, the suspension was degassed and purged with H₂ for 3 times, and then the mixture was stirred under H₂ atmosphere at 35° C. for 4 hr. LCMS (product: RT=0.470 min) showed the starting material was consumed completely. The reaction mixture was diluted with EtOH (20 ml), and then filtered through celite pad. The filter cake was rinsed with EtOH (30 ml*3), and the filtrate was concentrated under reduced pressure to give a product. 4 (900 mg, crude) was obtained as a yellow solid.

LCMS: RT=0.470 min, MS cal.: 212.6, [M+H]⁺=213.0

¹H NMR (400 MHz, CHCl3-d) δ=7.70 (d, J=8.4 Hz, 1H), 6.88 (d, J=8.4 Hz, 1H), 4.79 (t, J=8.8 Hz, 2H), 3.91 (s, 3H), 3.28 (t, J=8.8 Hz, 2H)

### General Procedure for Preparation of Intermediate 5:

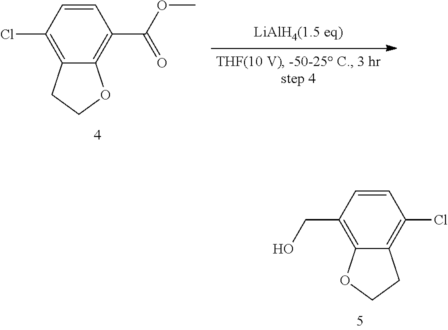

Equip a 25 mL three-necked round bottom flask, thermometer, N₂ balloon. THF (4 mL) was charged to the three-necked round bottom flask, then 4 (200 mg, 940.60 μmol, 1 eq) was added to the mixture at 25° C. At −50° C. (inner temperature), LiAlH₄ (2.5 M, 188.12 μL, 0.5 eq) was added dropwise to the reaction mixture. After the addition, the mixture was stirred at −50° C. for 1 hr. After monitoring, the reaction was not consumed completely. Then LiAlH₄ (2.5 M, 188.12 μL, 0.5 eq) was added dropwise to the reaction mixture. After the addition, the mixture was stirred at −50° C. for 1 hr. After monitoring, the reaction was not consumed completely. Then LiAlH₄ (2.5 M, 188.12 μL, 0.5 eq) was added dropwise to the reaction mixture. After the addition, the mixture was stirred at −50° C. for 1 hr. TLC (Petroleum ether:Ethyl acetate=3:1, R_{f}=0.21) indicated it was consumed completely and one new spot formed. The reaction was clean according to TLC. The reaction mixture was added to (10 mL) H₂O at 0° C. The mixture was extracted by EtOAc (10 mL*3). Then organic phase was combined and washed by brine (10 mL*2), dried with Na₂SO₄, filtered. The organic phase was concentrated under reduced pressure at 45° C. to give a residue. The crude 5 (160 mg, crude) was used into the next step without further purification as a yellow solid.

¹H NMR (400 MHz, CHCl3-d) δ=7.05 (d, J=8.0 Hz, 1H), 6.83 (d, J=8.0 Hz, 1H), 4.67 (t, J=8.8 Hz, 2H), 4.63 (s, 2H), 3.31-3.21 (m, 2H)

### General Procedure for Preparation of Compound 6:

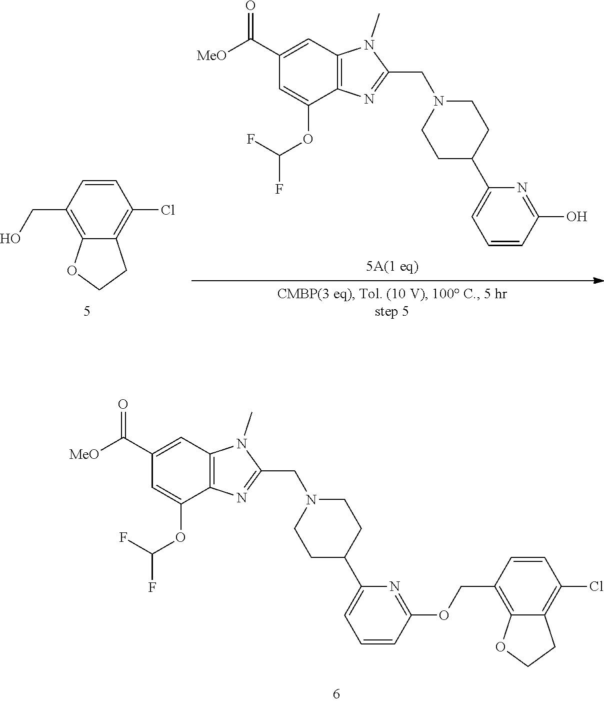 [SMILES: OCc1ccc(Cl)c2c1OCC2 ; COC(=O)c1cc(OC(F)F)c2nc(CN3CCC(c4cccc(O)n4)CC3)n(C)c2c1 ; COC(=O)c1cc(OC(F)F)c2nc(CN3CCC(c4cccc(OCc5ccc(Cl)c6c5OCC6)n4)CC3)n(C)c2c1]

A mixture of 5A (100 mg, 223.99 μmol, 1 eq), 5 (62.03 mg, 335.99 μmol, 1.5 eq), CMBP (162.18 mg, 671.97 μmol, 3 eq) in Toluene (1 mL) was degassed and purged with N₂ for 3 times, and then the mixture was stirred at 100° C. for 5 hr under N₂ atmosphere. LCMS (product: RT=1.760 min) showed the starting material was consumed completely. The reaction mixture was diluted with H₂O (10 mL) and extracted with EtOAc (15 mL*3). The combined organic layers were washed with brine (10 mL*2), dried over Na₂SO₄, filtered, and concentrated under reduced pressure to give a residue. The residue was purified by column chromatography (SiO₂, Petroleum ether/Ethyl acetate=100/1 to 0/1). 6 (70 mg, 114.18 μmol, 50.98% yield) was obtained as a yellow solid.

LCMS: RT=1.762 min, MS cal.: 613.1, [M+H]⁺=613.1

¹H NMR (400 MHz, CHCl3-d) δ=7.99 (d, J=0.8 Hz, 1H), 7.72 (s, 1H), 7.48 (br t, J=7.6 Hz, 1H), 7.32 (s, 1H), 7.21 (br d, J=8.4 Hz, 1H), 7.13 (s, 1H), 6.82 (d, J=8.4 Hz, 1H), 6.72 (br d, J=7.2 Hz, 1H), 6.61 (br d, J=8.0 Hz, 1H), 5.31 (d, J=2.4 Hz, 2H), 4.67 (t, J=8.8 Hz, 2H), 4.01 (br s, 3H), 3.97 (s, 3H), 3.91 (br d, J=7.2 Hz, 2H), 3.27 (t, J=8.8 Hz, 3H), 2.97 (br s, 2H), 2.65 (br d, J=9.2 Hz, 1H), 2.30 (br d, J=9.6 Hz, 2H), 2.00-1.78 (m, 4H)

### General Procedure for Preparation of Compound 331 (7):

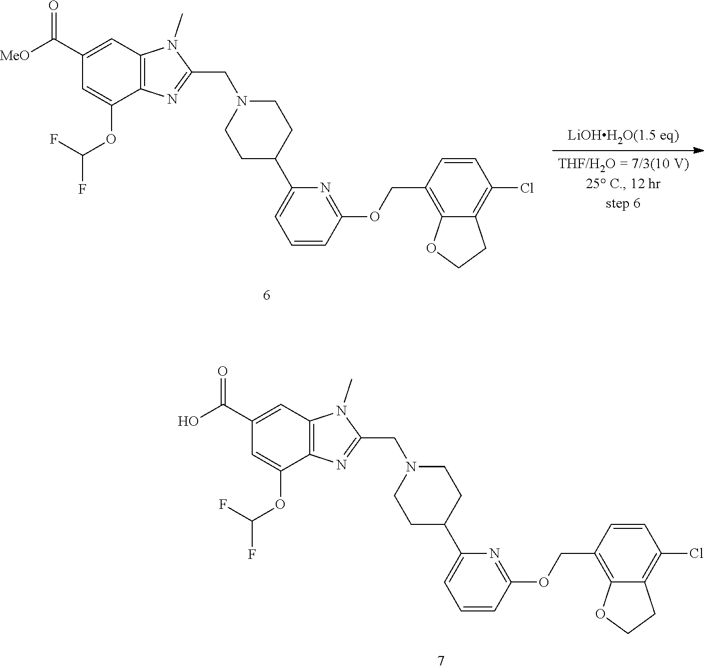 [SMILES: COC(=O)c1cc(OC(F)F)c2nc(CN3CCC(c4cccc(OCc5ccc(Cl)c6c5OCC6)n4)CC3)n(C)c2c1 ; Cn1c(CN2CCC(c3cccc(OCc4ccc(Cl)c5c4OCC5)n3)CC2)nc2c(OC(F)F)cc(C(=O)O)cc21]

A mixture of 6 (60 mg, 97.87 μmol, 1 eq), LiOH·H₂O (6.16 mg, 146.81 μmol, 1.5 eq) in THF (0.7 mL) and H₂O (0.3 mL) was degassed and purged with N₂ for 3 times, and then the mixture was stirred at 25° C. for 12 hr under N₂ atmosphere. LCMS (product: RT=1.203 min) showed the starting material was consumed completely. The crude product was purified by reversed-phase HPLC (column: Waters Xbridge BEH C18 100*30 mm*10 um; mobile phase: [H₂O (10 mM NH₄HCO₃)-ACN]; gradient: 35%-65% B over 8.0 min). 2-((4-(6-((4-Chloro-2,3-dihydrobenzofuran-7-yl)methoxy)pyridin-2-yl)piperidin-1-yl)methyl)-4-(difluoromethoxy)-1-methyl-1H-benzo[d]imidazole-6-carboxylic acid (27.63 mg, 46.13 μmol, 47.13% yield) was obtained as a white solid.

LCMS: RT=2.824 min, MS cal.: 599.0, [M+H]⁺=599.2

HPLC: RT=12.392 min, purity=97.009%

¹H NMR (400 MHz, DMSO-d₆) δ=8.07 (d, J=1.2 Hz, 1H), 7.83 (s, 1H), 7.64 (s, 1H), 7.61 (t, J=7.2 Hz, 1H), 7.45 (s, 1H), 7.23 (d, J=8.4 Hz, 1H), 6.88-6.82 (m, 2H), 6.63 (d, J=8.0 Hz, 1H), 5.21 (s, 2H), 4.65 (t, J=8.8 Hz, 2H), 3.96 (s, 3H), 3.86 (s, 2H), 3.26-3.16 (m, 3H), 2.96 (br d, J=11.2 Hz, 2H), 2.66-2.54 (m, 1H), 2.29-2.18 (m, 2H), 1.88-1.68 (m, 4H)

### Example A68—Preparation of Compound 298. 2-((4-(6-((4-Cyano-2-fluorobenzyl)oxy)pyridin-2-yl)piperidin-1-yl)methyl)-4-ethoxy-1-methyl-1H-benzo[d]imidazole-6-carboxylic acid (Compound 298) (3)

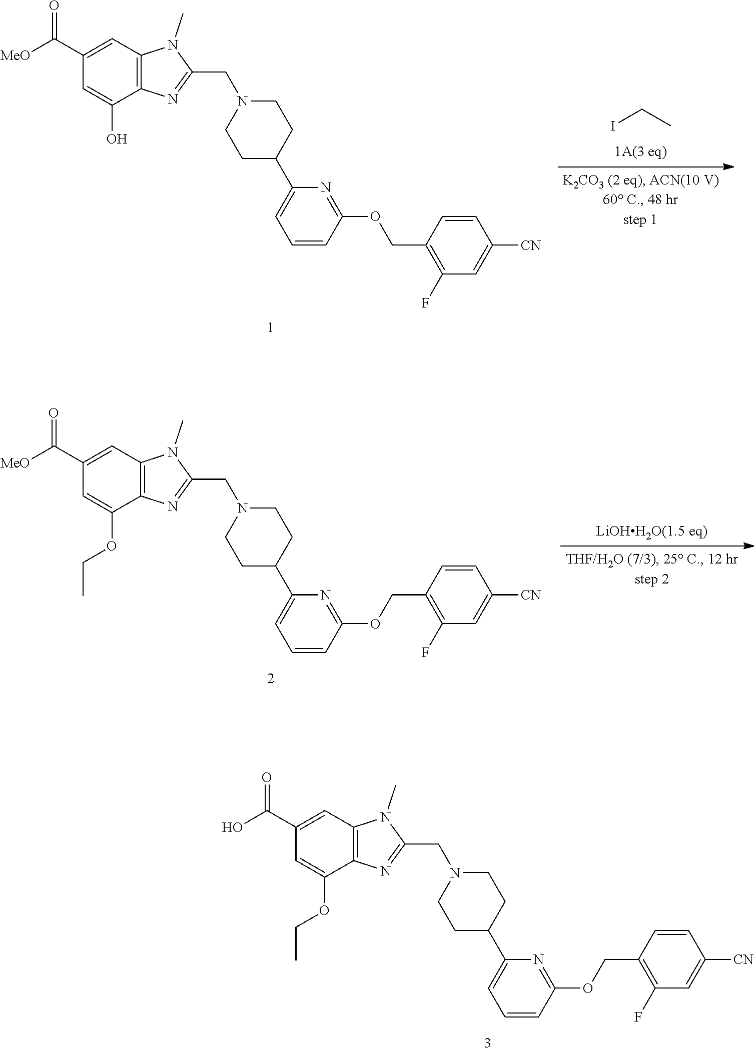

### General Procedure for Preparation of Intermediate 2:

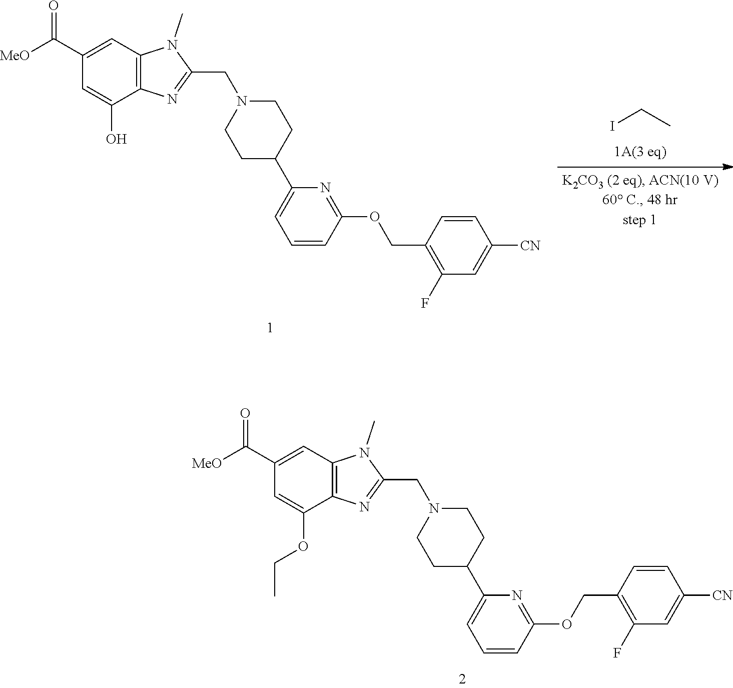

To a solution of 1 (100 mg, 188.84 μmol, 1 eq) in ACN (1 mL) was added K₂CO₃ (52.20 mg, 377.67 μmol, 2 eq) and 1A (88.36 mg, 566.51 μmol, 45.31 μL, 3 eq). The mixture was stirred at 60° C. for 48 hr. LCMS showed 1 was consumed completely and one main peak with desired mass was detected. The reaction mixture was diluted with H₂O (10 mL) and extracted with EtOAc (10 mL*3). The combined organic layers were washed with brine (10 mL*3), dried over Na₂SO₄, filtered and concentrated under reduced pressure to give a residue. The residue was purified by column chromatography (SiO₂, Petroleum ether/Ethyl acetate=10/1 to 0/1). 2 (80 mg, 143.47 μmol, 75.98% yield) was obtained as a yellow solid.

LCMS: RT=1.524 min, MS cal.: 557.2, [M+H]⁺=358.2

¹H NMR (400 MHz, CHCl3-d) δ=7.77 (d, J=0.8 Hz, 1H), 7.63 (br t, J=7.6 Hz, 1H), 7.57-7.49 (m, 1H), 7.44 (br d, J=8.0 Hz, 1H), 7.41-7.34 (m, 2H), 6.76 (d, J=7.2 Hz, 1H), 6.65 (d, J=8.4 Hz, 1H), 5.50 (s, 2H), 4.34 (q, J=7.2 Hz, 2H), 4.08-3.81 (m, 8H), 3.08-2.89 (m, 2H), 2.61 (br d, J=4.4 Hz, 1H), 2.41-2.21 (m, 2H), 1.91-1.70 (m, 4H), 1.57 (t, J=7.0 Hz, 3H)

### General Procedure for Preparation of Compound 298 (3):

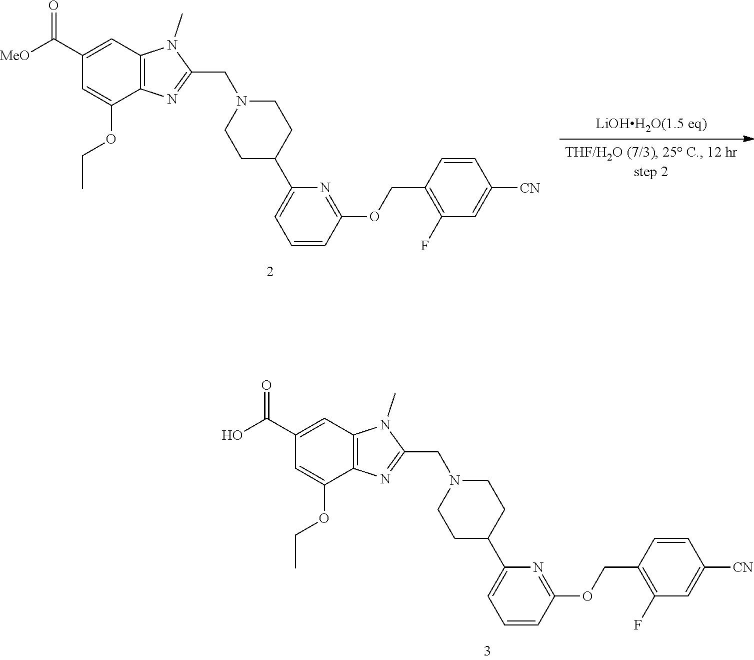 [SMILES: CCOc1cc(C(=O)OC)cc2c1nc(CN1CCC(c3cccc(OCc4ccc(C#N)cc4F)n3)CC1)n2C ; CCOc1cc(C(=O)O)cc2c1nc(CN1CCC(c3cccc(OCc4ccc(C#N)cc4F)n3)CC1)n2C]

A mixture of 2 (60 mg, 107.60 μmol, 1 eq), LiOH·H₂O (6.77 mg, 161.40 μmol, 1.5 eq) in THF (0.7 mL) and H₂O (0.3 mL) was degassed and purged with N₂ for 3 times, and then the mixture was stirred at 25° C. for 12 hr under N₂ atmosphere. LCMS showed 2 was consumed completely and one main peak with desired mass was detected. The reaction mixture was added with H₂O (10 mL) and extracted with EtOAc (10 mL*3). The combined organic layers were washed with brine (10 mL*3), dried over Na₂SO₄, filtered and concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 20%-50% B over 8.0 min). 2-((4-(6-((4-Cyano-2-fluorobenzyl)oxy)pyridin-2-yl)piperidin-1-yl)methyl)-4-ethoxy-1-methyl-1H-benzo[d]imidazole-6-carboxylic acid (29.14 mg, 53.61 μmol, 49.82% yield) was obtained as a white solid.

LCMS: RT=2.571 min, MS cal.: 543.2, [M+H]⁺=544.3

HPLC: RT=10.316 min, purity: 97.89%

¹H NMR (400 MHz, MeOH-d4) δ=7.85 (d, J=1.2 Hz, 1H), 7.67 (s, 1H), 7.62-7.51 (m, 3H), 7.43 (d, J=1.2 Hz, 1H), 6.83 (d, J=7.2 Hz, 1H), 6.68 (d, J=8.0 Hz, 1H), 5.51 (s, 2H), 4.29 (d, J=7.2 Hz, 2H), 3.98 (s, 3H), 3.93 (s, 2H), 3.08-3.00 (m, 2H), 2.71-2.58 (m, 1H), 2.41-2.30 (m, 2H), 1.89-1.79 (m, 4H), 1.53 (t, J=7.2 Hz, 3H)

### Example A69—Preparation of Compound 332 and Compound 333. (S)-2-((4-(3-(4-chloro-2-fluorophenyl)-2,3-dihydrobenzo[b][1,4]dioxin-5-yl)piperidin-1-yl)methyl)-4-(2-fluoroethoxy)-1-methyl-1H-benzo[d]imidazole-6-carboxylic acid (Compound 332) (9A) & (R)-2-((4-(3-(4-chloro-2-fluorophenyl)-2,3-dihydrobenzo[b][1,4]dioxin-5-yl)piperidin-1-yl)methyl)-4-(2-fluoroethoxy)-1-methyl-1H-benzo[d]imidazole-6-carboxylic acid (Compound 333) (9B)

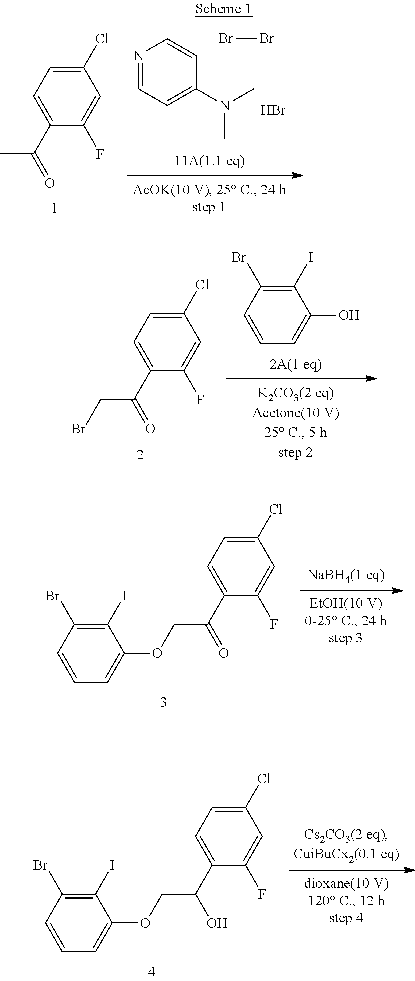

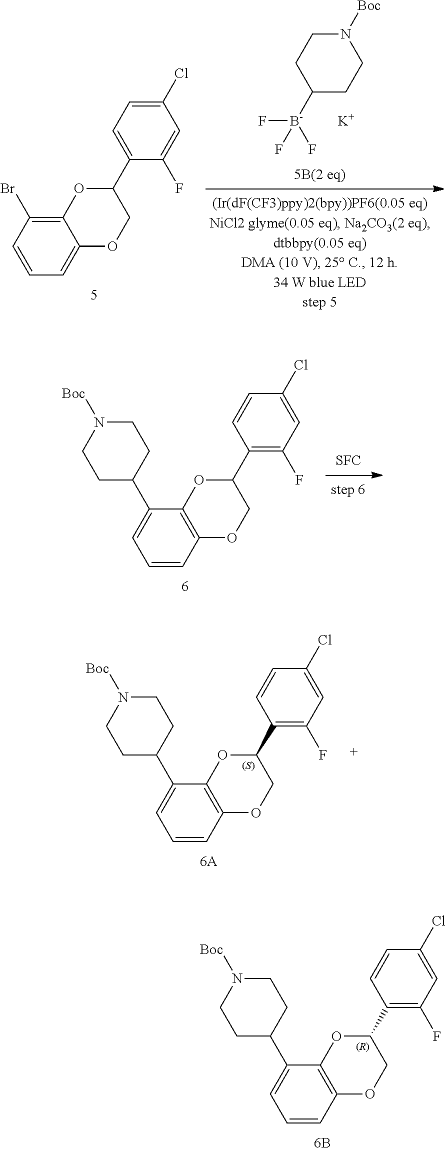

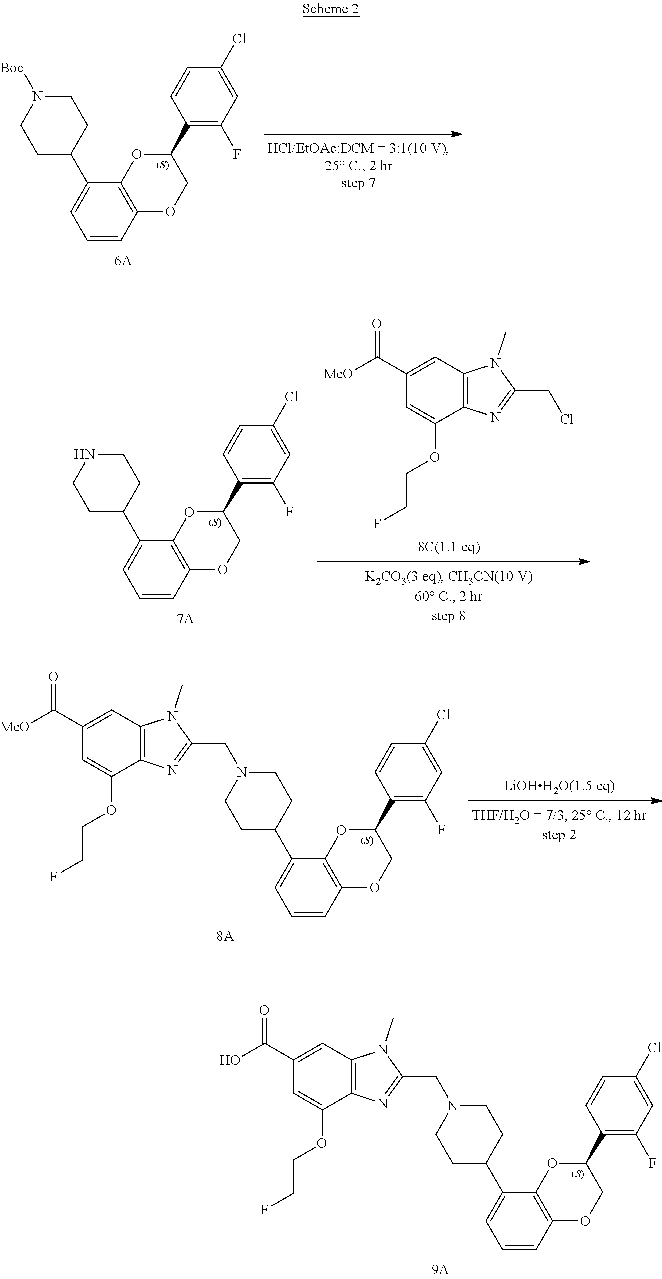

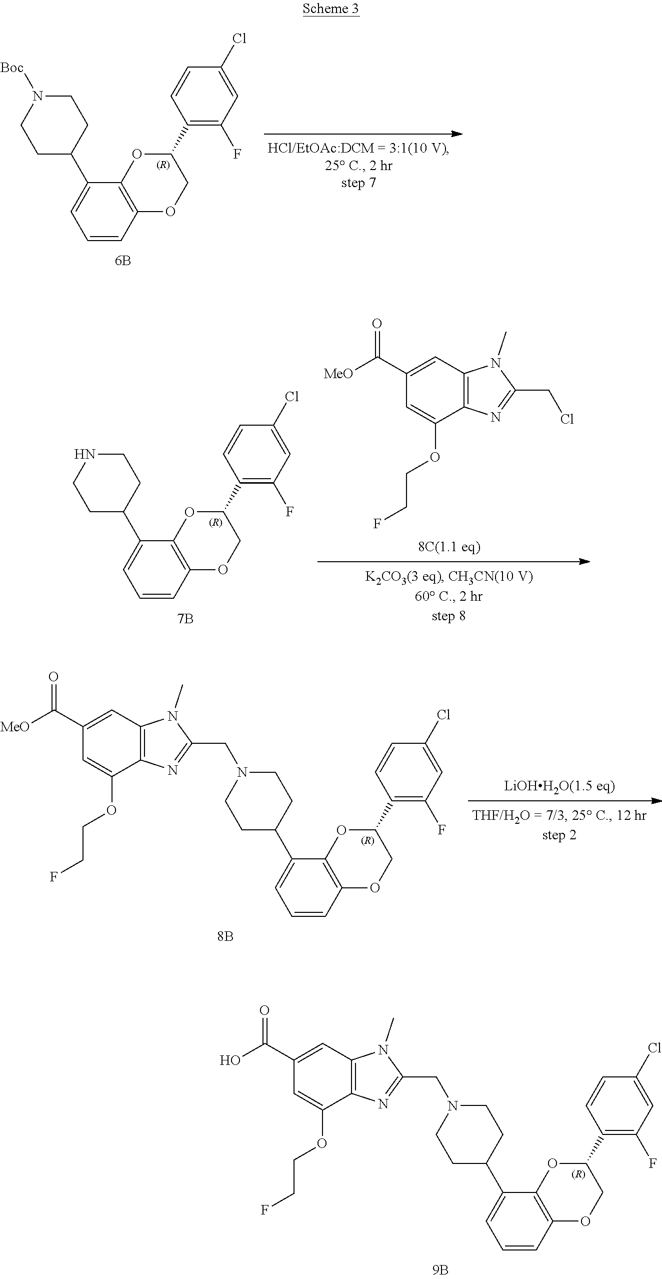

### General Procedure for Preparation of Scheme 1, Intermediate 2:

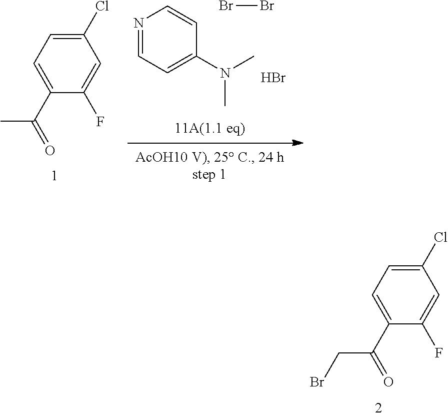

To a solution of 1 (4 g, 23.18 mmol, 1 eq) in AcOH (40 mL) was added 11A (9.25 g, 25.49 mmol, 1.1 eq). The mixture was stirred at 25° C. for 24 hr. TLC (Petroleum ether/Ethyl acetate=5:1) indicated 1 was completely and one new spot formed. The reaction was clean according to TLC. The mixture was poured into water (30 ml) at 25° C. Solid was precipitate out. It was filtered, then the solid was collected and dissolved in Ethyl acetate 10 mL, washed with water 5 mL, brine 5 mL, dried over Na₂SO₄, filtered and the filtrate was concentrated to give a crude product. 1 (9.5 g, 37.36 mmol, 80.60% yield, 98.9% purity) was obtained as a white solid.

¹HNMR (400 MHz, CHCl3-d) δ=7.94 (t, J=8.4 Hz, 1H), 7.33-7.29 (m, 1H), 7.25 (dd, J=2.0, 10.8 Hz, 1H), 4.51 (d, J=2.4 Hz, 2H)

### General Procedure for Preparation of Scheme 1, Intermediate 3:

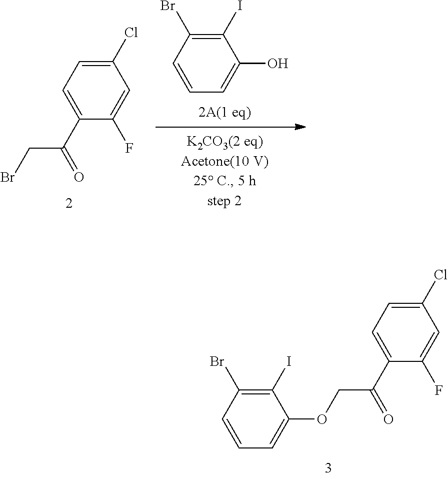 [SMILES: O=C(CBr)c1ccc(Cl)cc1F ; Oc1cccc(Br)c1I ; O=C(COc1cccc(Br)c1I)c1ccc(Cl)cc1F]

To a solution of 2 (9 g, 35.79 mmol, 1 eq) and 2A (10.70 g, 35.79 mmol, 1 eq) in Acetone (90 mL) was added K₂CO₃ (9.89 g, 71.58 mmol, 2 eq). The mixture was stirred at 25° C. for 5 hr. TLC (Petroleum ether/Ethyl acetate=5:1) indicated 2 was completely and one new spot formed. The reaction was clean according to TLC. The reaction mixture was added to H₂O 50 mL at 20° C.

The mixture was extracted by Ethyl acetate (50 mL*3). Then organic phase was combined and washed by brine (20 mL), dried with Na₂SO₄, filtered. The organic phase was concentrated under reduced pressure at 30° C. to give a residue. The residue was purified by column chromatography (SiO₂, Petroleum ether/Ethyl acetate=100/0 to 90/1). 3 (15.87 g, 31.44 mmol, 87.84% yield) was obtained as a yellow solid.

### General Procedure for Preparation of Scheme 1, Intermediate 4:

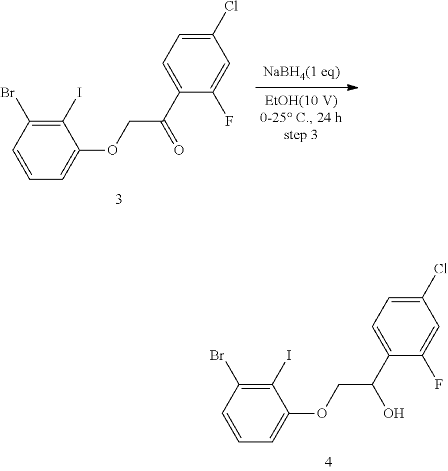 [SMILES: O=C(COc1cccc(Br)c1I)c1ccc(Cl)cc1F ; OC(COc1cccc(Br)c1I)c1ccc(Cl)cc1F]

At 20° C., 3 (7.9 g, 16.83 mmol, 1 eq) in EtOH (79 mL) was charged to the three round bottom flask at 25° C. NaBH₄ (1.27 g, 33.65 mmol, 1 eq) was added to the mixture at 0° C. After the addition, the mixture was stirred at 25° C. for 24 hr. TLC (Petroleum ether/Ethyl acetate=5:1) indicated 3 was completely and one new spot formed. The reaction was clean according to TLC. After 1 hr, 20 mL H₂O was added to the reaction mixture at 0° C. The mixture was diluted with H₂O (50 mL) and extracted by Ethyl acetate (60 mL*3). Then organic phase was combined and washed by brine (30 mL*2), dried with Na₂SO₄, filtered. The organic phase was concentrated under reduced pressure at 40° C. to give a residue. The residue was purified by column chromatography (SiO₂, Petroleum ether/Ethyl acetate=100/0 to 96/4). And the crude product was also purified by pre-HPLC (column: Welch Xtimate C18 250*100 mm #10 um; mobile phase: [H₂O (10 mM NH₄HCO₃)-ACN]; gradient: 40%-75% B over 20.0 min). 4 (5.6 g, 11.16 mmol, 66.35% yield, 94% purity) was obtained as a white oil.

¹HNMR (400 MHz, DMSO-d₆) δ=7.73 (t, J=8.0 Hz, 1H), 7.44 (dd, J=1.6, 10.0 Hz, 1H), 7.40-7.28 (m, 3H), 7.03 (br d, J=7.6 Hz, 1H), 5.93 (d, J=4.8 Hz, 1H), 5.26 (q, J=4.8 Hz, 1H), 4.29-4.16 (m, 2H)

### General Procedure for Preparation of Scheme 1, Intermediate 5:

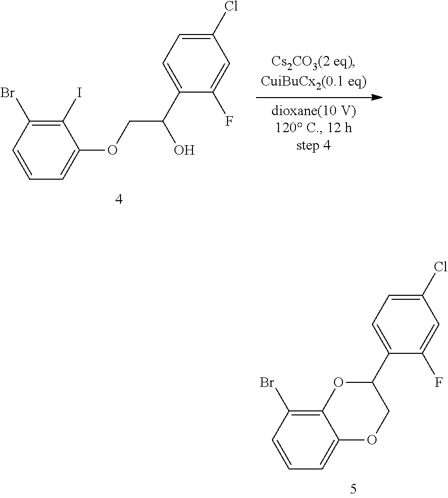 [SMILES: OC(COc1cccc(Br)c1I)c1ccc(Cl)cc1F ; Fc1cc(Cl)ccc1C1COc2cccc(Br)c2O1]

To a solution of 4 (1.5 g, 3.18 mmol, 1 eq) in dioxane (15 mL) was added Cs₂CO₃ (2.07 g, 6.36 mmol, 2 eq) and bis[(1Z)-2-methyl-1-(2-oxocyclohexylidene) propoxy] copper (126.62 mg, 318.14 μmol, 0.1 eq). The mixture was stirred at 120° C. for 12 hr. TLC (Petroleum ether/Ethyl acetate=5:1) indicated 4 was completely and one new spot formed. The reaction was clean according to TLC. The reaction mixture was added to H₂O 20 mL at 20° C. The mixture was extracted by Ethyl acetate (20 mL*3). Then organic phase was combined and washed by brine (10 mL), dried with Na₂SO₄, filtered. The organic phase was concentrated under reduced pressure at 30° C. to give a residue. The residue was purified by column chromatography (SiO₂, Petroleum ether/Ethyl acetate=100/0 to 100/0). 5 (865 mg, 2.39 mmol, 75.18% yield) was obtained as a green solid.

¹HNMR (400 MHz, DMSO-d₆) δ=7.64-7.55 (m, 2H), 7.46 (dd, J=2.0, 8.4 Hz, 1H), 7.23 (dd, J=1.2, 8.0 Hz, 1H), 7.03-7.00 (m, 1H), 6.88 (t, J=8.0 Hz, 1H), 5.62 (dd, J=2.4, 8.0 Hz, 1H), 4.55 (dd, J=2.4, 11.6 Hz, 1H), 4.23 (dd, J=8.0, 11.6 Hz, 1H)

### General Procedure for Preparation of Scheme 1, Intermediate 6:

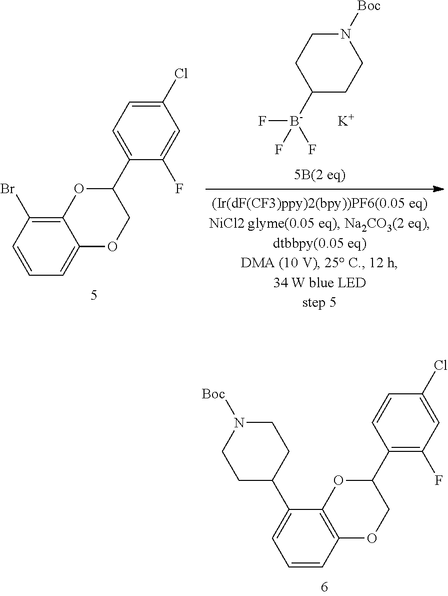 [SMILES: Fc1cc(Cl)ccc1C1COc2cccc(Br)c2O1 ; CC(C)(C)OC(=O)N1CCC([B-](F)(F)F)CC1 ; [K+] ; CC(C)(C)OC(=O)N1CCC(c2cccc3c2OC(c2ccc(Cl)cc2F)CO3)CC1]

To a solution of 5 (500 mg, 1.46 mmol, 1 eq) and 5B (847.44 mg, 2.91 mmol, 2 eq) in DMA (15 mL) was added dichloronickel; 1,2-dimethoxyethane (15.99 mg, 72.76 μmol, 0.05 eq) and Na₂CO₃ (308.49 mg, 2.91 mmol, 2 eq) and 4-tert-butyl-2-(4-tert-butyl-2-pyridyl)pyridine (19.53 mg, 72.76 μmol, 0.05 eq) and bis[3,5-difluoro-2-[5-(trifluoromethyl)-2-pyridyl]phenyl]iridium(1+); 2-(2-pyridyl)pyridine; hexafluorophosphate (44.08 mg, 43.66 μmol, 0.03 eq). The mixture was stirred at 25° C. for 12 hr at 34W blue LED. LCMS showed 5 was consumed completely and one main peak with desired m/z was detected. TLC (Petroleum ether/Ethyl acetate=5:1) indicated 5 was completely and one new spot formed. The reaction was clean according to TLC. The reaction mixture was added to H₂O 30 mL at 20° C. The mixture was extracted by Ethyl acetate (30 mL*3). Then organic phase was combined and washed by brine (30 mL), dried with Na₂SO₄, filtered. The organic phase was concentrated under reduced pressure at 30° C. to give a residue. The residue was purified by column chromatography (SiO₂, Petroleum ether/Ethyl acetate=100/1 to 50/1). 6 (0.94 g, 1.99 mmol, 45.66% yield) was obtained as a white solid.

LCMS: RT=1.810 min, MS cal.: 447.16, 449.16, [M−55]⁺=391.9

¹HNMR (400 MHz, DMSO-d₆) δ=7.61-7.48 (m, 2H), 7.47-7.34 (m, 1H), 6.87-6.75 (m, 3H), 5.47 (br d, J=6.0 Hz, 1H), 4.44 (dd, J=2.0, 11.6 Hz, 1H), 4.16-3.98 (m, 3H), 2.99 (br t, J=12.0 Hz, 1H), 2.76 (br s, 2H), 1.80-1.65 (m, 2H), 1.61-1.44 (m, 2H), 1.38 (s, 9H)

### General Procedure for Preparation of Scheme 1, Intermediate 6A&6B:

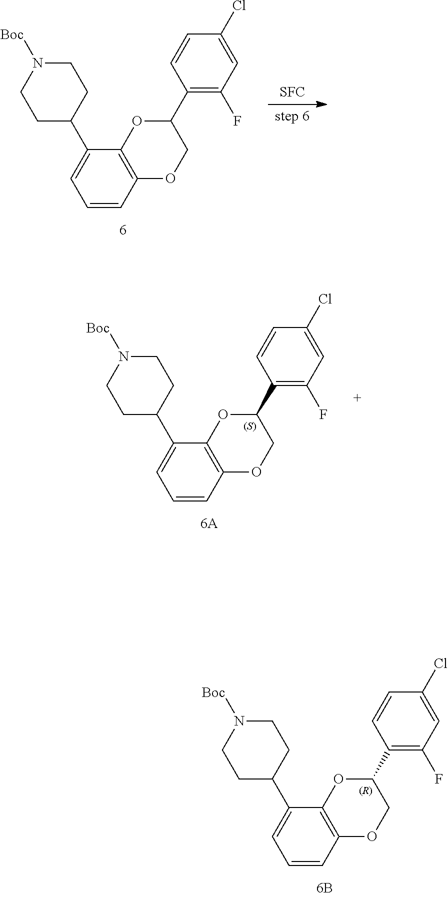

The 6 was separated by SFC (column: DAICEL CHIRALCEL OJ (250 mm*50 mm, 10 um); mobile phase: [CO₂-MeOH]; B %: 25%, isocratic elution mode). 6A (380 mg, 848.35 μmol, 42.22% yield) and 6B (400 mg, 848.35 μmol, 44.44% yield) was obtained as a white solid.

¹HNMR (400 MHz, DMSO-d₆) (6A) δ=7.62-7.54 (m, 2H), 7.44 (dd, J=2.0, 8.4 Hz, 1H), 6.90-6.81 (m, 3H), 5.50 (dd, J=2.0, 7.6 Hz, 1H), 4.47 (dd, J=2.4, 11.6 Hz, 1H), 4.14 (dd, J=8.0, 11.6 Hz, 1H), 4.10-4.03 (m, 2H), 3.07-2.97 (m, 1H), 2.93-2.67 (m, 2H), 1.84-1.68 (m, 2H), 1.64-1.44 (m, 3H), 1.41 (s, 9H)

¹HNMR (400 MHz, DMSO-d₆) (6B) δ=7.57 (dd, J=2.0, 10.4 Hz, 1H), 7.52 (t, J=8.4 Hz, 1H), 7.40 (dd, J=1.6, 8.4 Hz, 1H), 6.86-6.77 (m, 3H), 5.47 (dd, J=2.0, 8.0 Hz, 1H), 4.44 (dd, J=2.4, 11.6 Hz, 1H), 4.11 (dd, J=8.0, 11.6 Hz, 1H), 4.07-3.96 (m, 2H), 3.05-2.93 (m, 1H), 2.88-2.64 (m, 2H), 1.80-1.65 (m, 2H), 1.61-1.43 (m, 2H), 1.38 (s, 9H)

### General Procedure for Preparation of Scheme 2, Intermediate 7A:

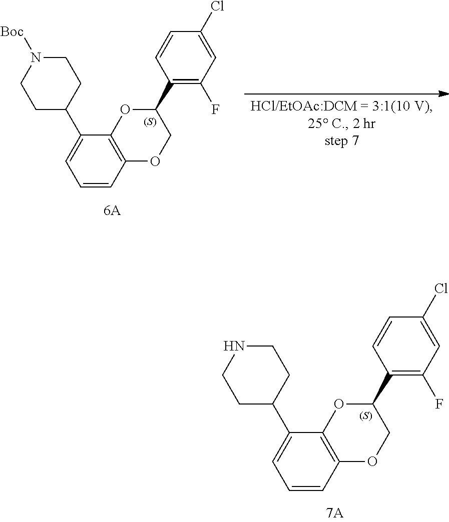

To a solution of 6A (340 mg, 759.05 μmol, 1 eq) in DCM (3 mL) was added HCl/EtOAc (9 mL). The mixture was stirred at 25° C. for 2 hr. LC-MS showed 6A was consumed completely and desired mass was detected. The organic phase was concentrated under reduced pressure at 40° C. to give a residue. The reaction mixture was concentrated to give 7A (290 mg, 754.68 μmol, 99.42% yield, HCl) was obtained as an orange oil.

LCMS: RT=1.185 min, MS cal.: 347.1, [M+H]⁺=348.1

¹H NMR (400 MHz, CHCl3-d) δ=7.41-7.33 (m, 1H), 7.26-7.22 (m, 1H), 7.21-7.16 (m, 1H), 6.95-6.85 (m, 2H), 6.82 (br d, J=7.2 Hz, 1H), 5.47-5.38 (m, 1H), 4.41 (dd, J=2.0, 11.3 Hz, 1H), 4.02 (dd, J=8.4, 11.2 Hz, 1H), 3.63 (br s, 2H), 3.21 (br d, J=1.6 Hz, 1H), 3.07 (br s, 2H), 2.22-1.98 (m, 5H)

### General Procedure for Preparation of Scheme 2, Intermediate 8A:

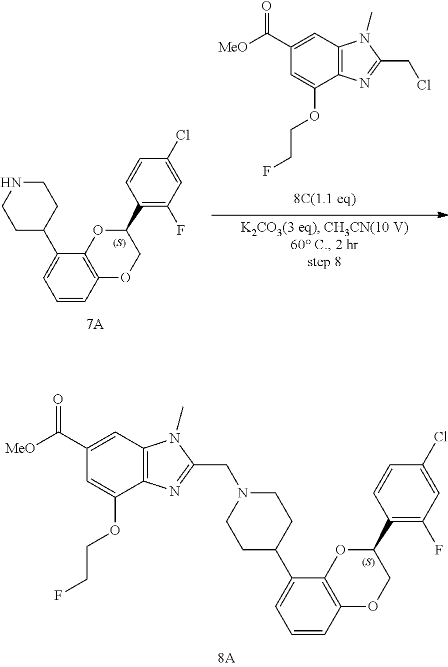

A mixture of 7A (106.71 mg, 354.86 μmol, 1 eq), 8C (150 mg, 390.35 μmol, 1.1 eq, HCl), K₂CO₃ (147.13 mg, 1.06 mmol, 3 eq) in ACN (2 mL) was degassed and purged with N₂ for 3 times, and then the mixture was stirred at 60° C. for 2 hr under N₂ atmosphere. LC-MS (Product, RT=1.674 min) showed 7A was consumed completely and one main peak with desired m/z or desired mass was detected. The reaction mixture was diluted with H₂O 20 mL and extracted with EtOAc 60 mL (20 mL*3). The combined organic layers were washed with Na₂SO₄ 15 mL, dried over NA₂SO₄, filtered and concentrated under reduced pressure to give a residue. The residue was purified by column chromatography (SiO₂, Petroleum ether/Ethyl acetate=10/1 to 0/1). 8A (210 mg, 343.10 μmol) was obtained as a white solid.

LCMS: RT=1.674 min, MS cal.: 611.20, [M+H]⁺=612.4

### General Procedure for Preparation of Compound 332 (9A):

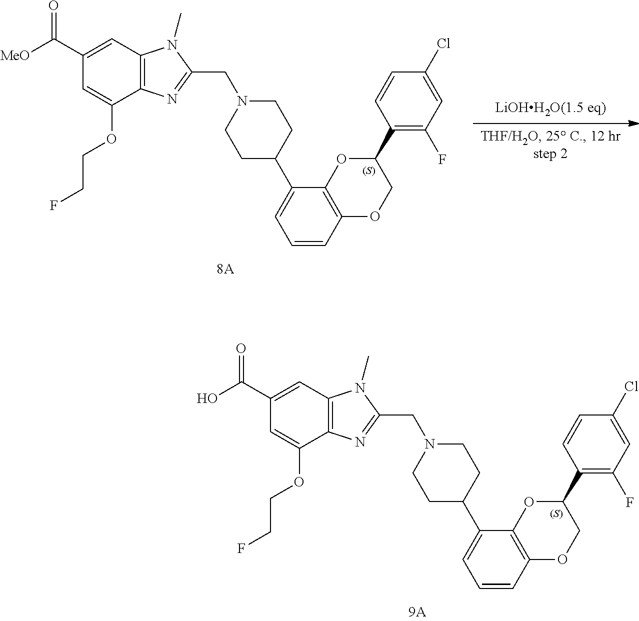

A mixture of 8A (150.00 mg, 245.07 μmol, 1 eq), LiOH·H₂O (15.43 mg, 367.61 μmol, 1.5 eq) in THF (1.4 mL) and H₂O (0.6 mL) was degassed and purged with N₂ for 3 times, and then the mixture was stirred at 30° C. for 12 hr under N₂ atmosphere. LC-MS (product, RT=1.191 min) showed 8A was consumed completely and one main peak with desired mass was detected. The reaction was purified directly. The residue was purified by prep-HPLC (column: Waters Xbridge Prep OBD C18 150*40 mm*10 um; mobile phase: [H₂O (10 mM NH₄HCO₃)-ACN]; gradient: 20%-70% B over 8.0 min). (S)-2-((4-(3-(4-Chloro-2-fluorophenyl)-2,3-dihydrobenzo[b][1,4]dioxin-5-yl)piperidin-1-yl)methyl)-4-(2-fluoroethoxy)-1-methyl-1H-benzo[d]imidazole-6-carboxylic acid (28.52 mg, 47.52 μmol, 19.39% yield) was obtained as a white solid.

LCMS: RT=2.399 min, MS cal.: 597.18, [M+1]⁺=598.

¹HNMR (400 MHz, DMSO) δ=7.82-7.73 (m, 1H), 7.57 (br d, J=10.0 Hz, 1H), 7.55-7.49 (m, 1H), 7.47-7.39 (m, 1H), 7.29 (s, 1H), 6.86-6.74 (m, 3H), 5.46 (br d, J=6.4 Hz, 1H), 4.82 (dt, J=48, 3.2 Hz, 2H), 4.52-4.42 (m, 3H), 4.09 (dd, J=8.0, 11.2 Hz, 1H), 3.86 (s, 3H), 3.82-3.73 (m, 2H), 2.91 (br d, J=10.0 Hz, 2H), 2.87-2.78 (m, 1H), 2.21-2.09 (m, 2H), 1.83-1.54 (m, 4H)

### General Procedure for Preparation of Scheme 2, Intermediate 7B:

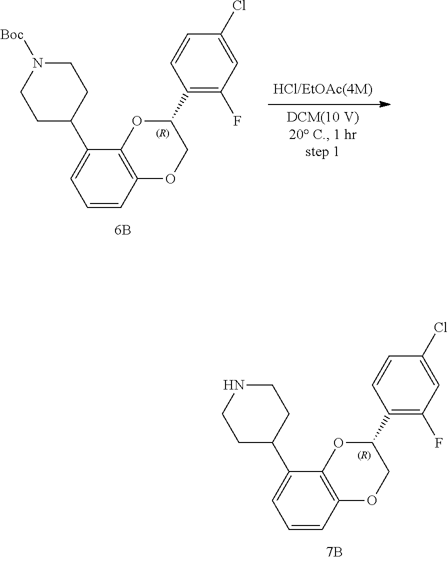

A mixture of 6B (360 mg, 803.70 μmol, 1 eq) in DCM (1 mL) and HCl/EtOAc (2.6 mL) was degassed and purged with N₂ for 3 times, and then the mixture was stirred at 20° C. for 1 hr under N₂ atmosphere. LCMS (Product, Rt=1.153 min) showed 95% of product 1 was detected. The reaction mixture was concentrated under reduced pressure to give a residue. Without purification. 7B (300 mg, crude) was obtained as a white solid.

LCMS: RT=1.217 min, MS cal.: 347.1/349.1, [M+1]⁺=347.9/349.9

### General Procedure for Preparation of Intermediate 8B:

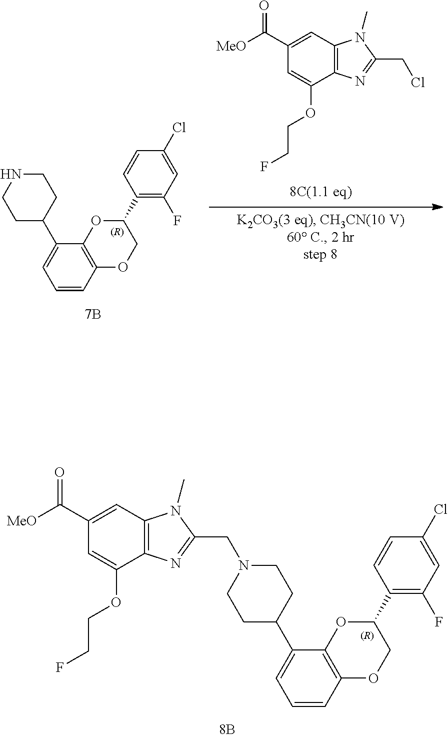

To a solution of 7B (71.14 mg, 236.58 μmol, 1 eq) in ACN (1 mL) was added K₂CO₃ (98.09 mg, 709.73 μmol, 3 eq) and 8C (synthesized from Int 6) (100 mg, 260.23 μmol, 1.1 eq, HCl). The mixture was stirred at 60° C. for 2 hr. TLC (PE/EtOAc-0/1, Rf=0.4) indicated 7B was consumed completely and one new spot formed. The reaction was clean according to TLC. The reaction mixture was diluted with H₂O 5 mL and extracted with EtOAc 15 mL (5 mL*3). The combined organic layers were washed with brine 10 mL, dried over Na₂SO₄, filtered and concentrated under reduced pressure to give a residue. The residue was purified by column chromatography (SiO₂, PE/EtOAc=1/0 to 1/1). Compound 8B (70 mg, 108.65 μmol, 45.93% total yield) was obtained as a white solid.

LCMS: RT=1.732 min, MS cal.: 611.2/612.2, [M−1]⁻=612.1/613.1

¹HNMR (400 MHz, DMSO-d6) δ=7.84 (s, 1H), 7.61-7.49 (m, 2H), 7.45-7.40 (m, 1H), 7.29-7.27 (m, 1H), 6.86-6.75 (m, 3H), 5.46 (br d, J=6.0 Hz, 1H), 4.82 (dt, J=47.6, 3.2 Hz, 2H), 4.55-4.42 (m, 3H), 4.09 (dd, J=8.0, 11.6 Hz, 1H), 3.88 (d, J=4.8 Hz, 5H), 3.91-3.85 (m, 1H), 3.80 (s, 2H), 2.95-2.87 (m, 2H), 2.87-2.79 (m, 1H), 2.22-2.07 (m, 2H), 1.83-1.53 (m, 4H)

### General Procedure for Preparation of Compound 333 (9B):

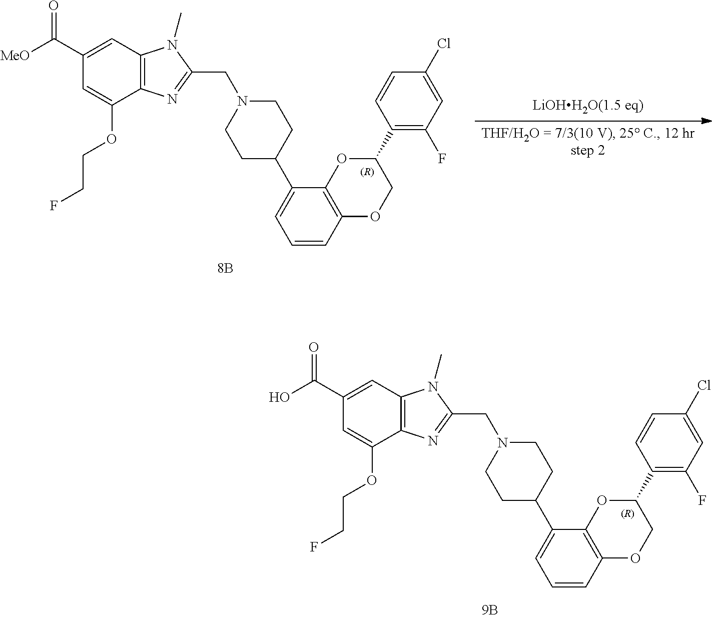

To a solution of 8B (60 mg, 98.03 μmol, 1 eq) in THF (0.7 mL) and H₂O (0.3 mL) was added LiOH·H₂O (6.17 mg, 147.04 μmol, 1.5 eq). The mixture was stirred at 25° C. for 12 hr. LCMS showed 8B was consumed completely and one main peak with desired mass was detected. The mixture was added THF 0.1 mL. The residue was purified by prep-HPLC (neutral condition: column: Waters Xbridge Prep OBD C18 150*40 mm*10 um; mobile phase: [H₂O (10 mM NH₄HCO₃)-ACN]; gradient: 20%-70% B over 8.0 min). (R)-2-((4-(3-(4-Chloro-2-fluorophenyl)-2,3-dihydrobenzo[b][1,4]dioxin-5-yl)piperidin-1-yl)methyl)-4-(2-fluoroethoxy)-1-methyl-1H-benzo[d]imidazole-6-carboxylic acid (26.09 mg, 43.19 μmol, 44.06% yield) was obtained as a white solid.

LCMS: RT=2.776 min, MS cal.: 597.2/598.2, [M+H]⁺=598.2/599.2

HPLC: RT=11.921 min

¹HNMR (400 MHz, DMSO-d6) δ=7.80-7.73 (m, 1H), 7.60-7.55 (m, 1H), 7.55-7.50 (m, 1H), 7.45-7.41 (m, 1H), 7.28 (s, 1H), 6.85-6.76 (m, 3H), 5.46 (dd, J=2.0, 8.0 Hz, 1H), 4.82 (dt, J=48, 3.6 Hz, 2H), 4.51-4.43 (m, 3H), 4.10 (dd, J=8.0, 11.4 Hz, 1H), 3.86 (s, 3H), 3.82-3.73 (m, 2H), 2.91 (br d, J=10.8 Hz, 2H), 2.87-2.80 (m, 1H), 2.25-2.07 (m, 2H), 1.84-1.53 (m, 4H)

### Example A70—Preparation of Compound 334 Compound 335. (S)-2-((4-(3-(4-Chloro-2-fluorophenyl)-2,3-dihydrobenzo[b][1,4]dioxin-5-yl)piperidin-1-yl)methyl)-4-(difluoromethoxy)-1-methyl-1H-benzo[d]imidazole-6-carboxylic acid (Compound 334) (10A) & (R)-2-((4-(3-(4-Chloro-2-fluorophenyl)-2,3-dihydrobenzo[b][1,4]dioxin-5-yl)piperidin-1-yl)methyl)-4-(difluoromethoxy)-1-methyl-1H-benzo[d]imidazole-6-carboxylic acid (Compound 335) (10B)

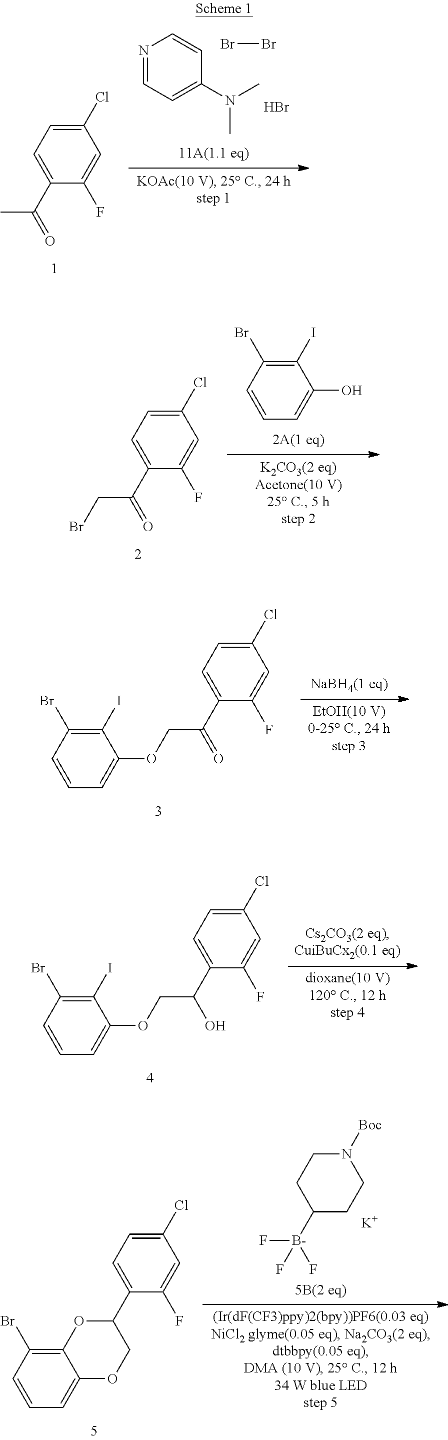

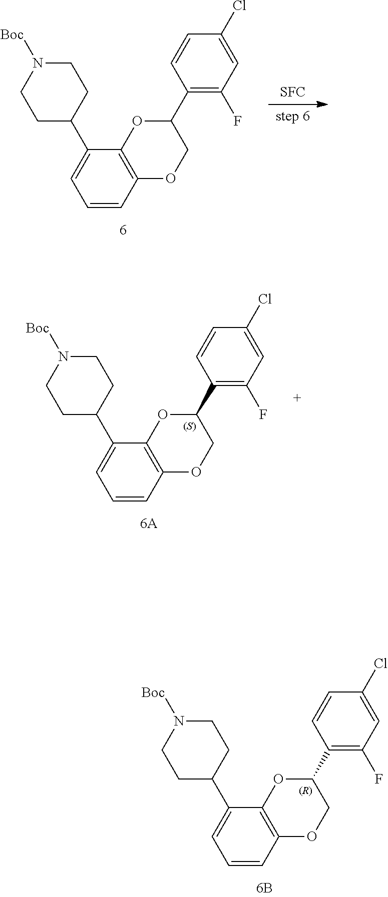

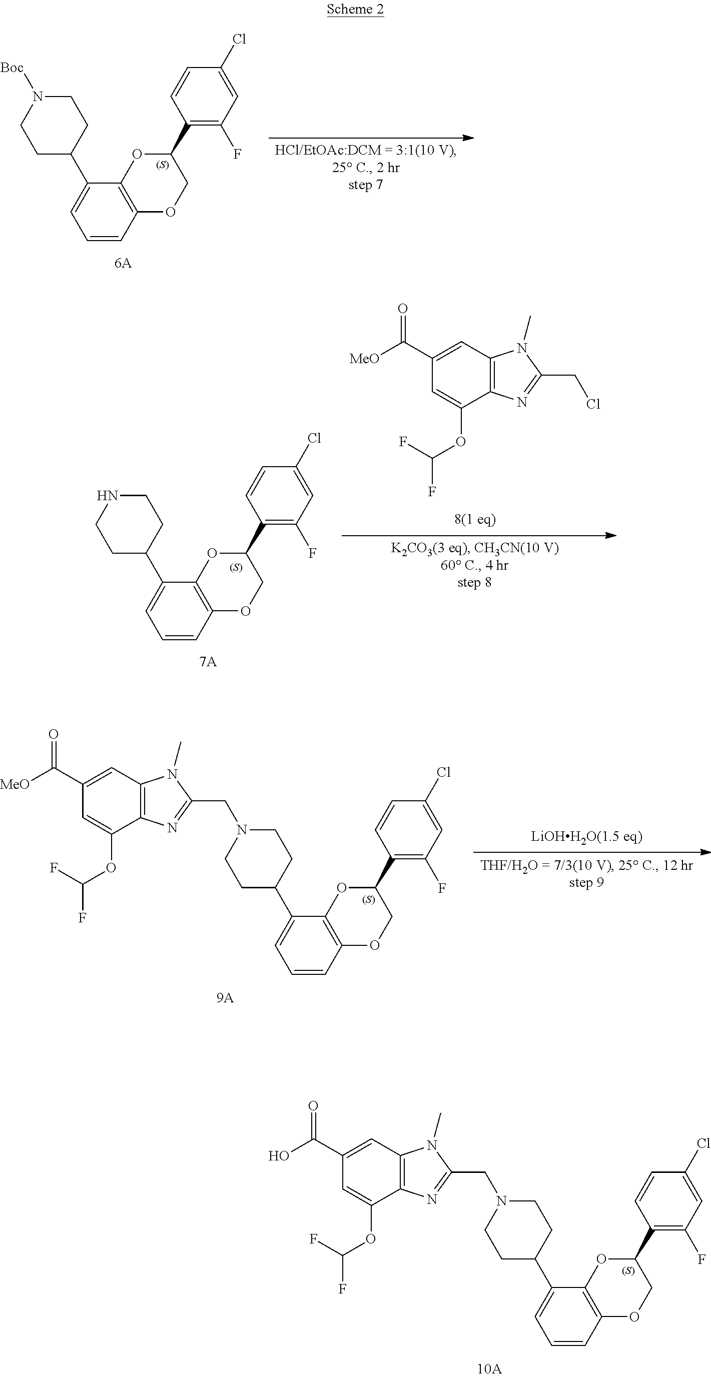

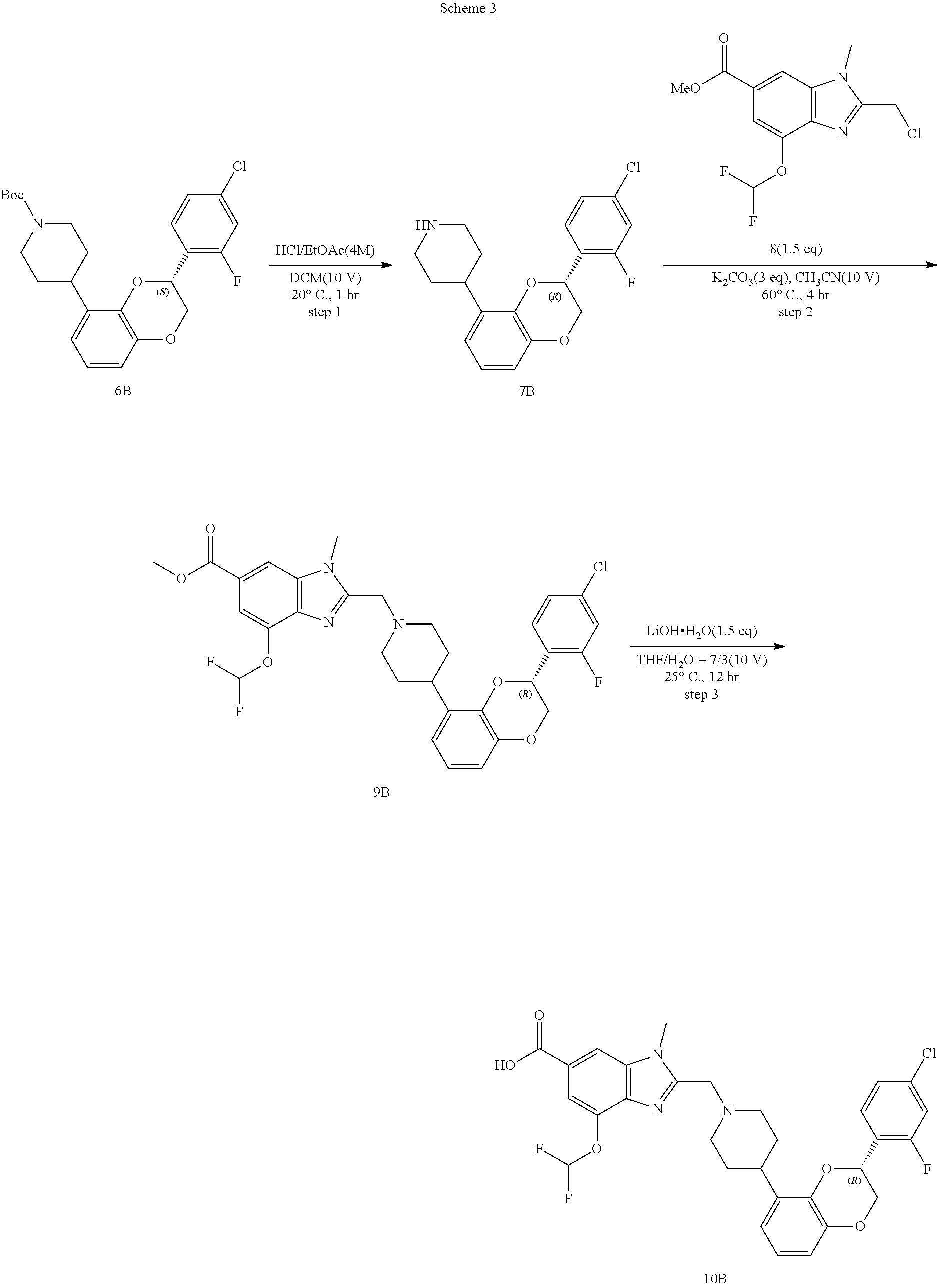

### General Procedure for Preparation of Intermediate 2:

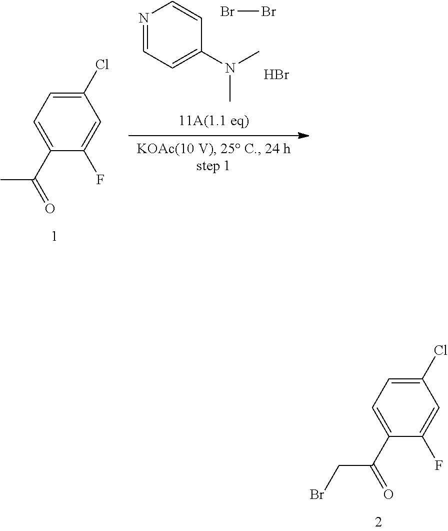

To a solution of 1 (4 g, 23.18 mmol, 1 eq) in KOAc (40 mL) was added 1A (9.25 g, 25.49 mmol, 1.1 eq). The mixture was stirred at 25° C. for 24 hr. TLC (Petroleum ether/Ethyl acetate=5:1) indicated 1 was completely and one new spot formed. The reaction was clean according to TLC. The mixture was poured into water (10 mL) at 0° C. Solid was precipitate out. And the crude product was triturated with H₂O (30 ml) at 25° C. for 30 min. 1 (9.5 g, 37.36 mmol, 80.60% yield, 98.9% purity) was obtained as a white solid.

¹H NMR (400 MHz, CHCl3-d) δ=7.94 (t, J=8.0 Hz, 1H), 7.33-7.29 (m, 1H), 7.25 (dd, J=2.0, 10.8 Hz, 1H), 4.51 (d, J=2.4 Hz, 2H)

### General Procedure for Preparation of Intermediate 3:

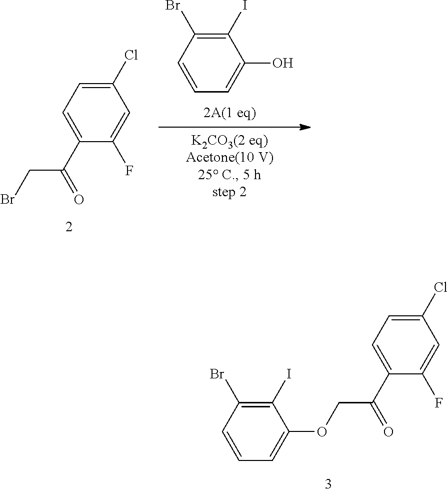

To a solution of 2 (9 g, 35.79 mmol, 1 eq) and 2A (10.70 g, 35.79 mmol, 1 eq) in Acetone (90 mL) was added K₂CO₃ (9.89 g, 71.58 mmol, 2 eq). The mixture was stirred at 25° C. for 5 hr. TLC

(Petroleum ether/Ethyl acetate=5:1) indicated 2 was completely and one new spot formed. The reaction was clean according to TLC. The reaction mixture was added to H₂O (50 mL) at 20° C. The mixture was extracted by Ethyl acetate (50 mL*3). Then organic phase was combined and washed by brine (20 mL), dried with Na₂SO₄, filtered. The organic phase was concentrated under reduced pressure at 30° C. to give a residue. The residue was purified by column chromatography (SiO₂, Petroleum ether/Ethyl acetate=100/0 to 90/1). 3 (15.87 g, 31.44 mmol, 87.84% yield, 93% purity) was obtained as a white solid.

¹H NMR (400 MHz, CHCl3-d) δ=7.87 (t, J=8.0 Hz, 1H), 7.25-7.22 (m, 2H), 7.20-7.18 (m, 1H), 7.07 (t, J=8.0 Hz, 1H), 6.52 (dd, J=1.2, 8.4 Hz, 1H), 5.16 (d, J=3.2 Hz, 2H)

### General Procedure for Preparation of Intermediate 4:

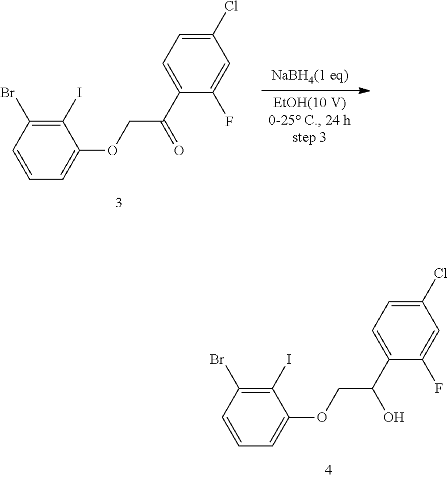 [SMILES: O=C(COc1cccc(Br)c1I)c1ccc(Cl)cc1F ; "EtOH(10 V)0-25° C., 24 hstep 3" ; "NaBH4(1 eq)" ; OC(COc1cccc(Br)c1I)c1ccc(Cl)cc1F]

3 (7.9 g, 16.83 mmol, 1 eq) in EtOH (79 mL) was charged to the three round bottom flask. NaBH₄ (1.27 g, 33.65 mmol, 1 eq) was added to the mixture at 0° C. After the addition, the mixture was stirred at 25° C. for 24 hr. TLC (Petroleum ether/Ethyl acetate=5:1) indicated 3 was completely and one new spot formed. The reaction was clean according to TLC. After 1 hr, 20 mL H₂O was added to the reaction mixture at 0° C. The mixture was diluted with H₂O (50 mL), and extracted by Ethyl acetate (60 mL*3). Then organic phase was combined and washed by brine (30 mL*2), dried with Na₂SO₄, filtered. The organic phase was concentrated under reduced pressure at 40° C. to give a residue. The residue was purified by column chromatography (SiO₂, Petroleum ether/Ethyl acetate=100/0 to 96/4). And the crude product was also purified by pre-HPLC (column: Welch Xtimate C18 250*100 mm #10 um; mobile phase: [H₂O (10 mM NH₄HCO₃)-ACN]; gradient: 40%-75% B over 20.0 min). 4 (5.6 g, 11.16 mmol, 66.35% yield, 94% purity) was obtained as a white oil.

¹H NMR (400 MHz, DMSO-d₆) δ=7.73 (t, J=8.0 Hz, 1H), 7.44 (dd, J=1.6, 10.0 Hz, 1H), 7.40-7.28 (m, 3H), 7.03 (br d, J=7.6 Hz, 1H), 5.93 (d, J=4.8 Hz, 1H), 5.26 (q, J=5.2 Hz, 1H), 4.29-4.16 (m, 2H)

### General Procedure for Preparation of Intermediate 5:

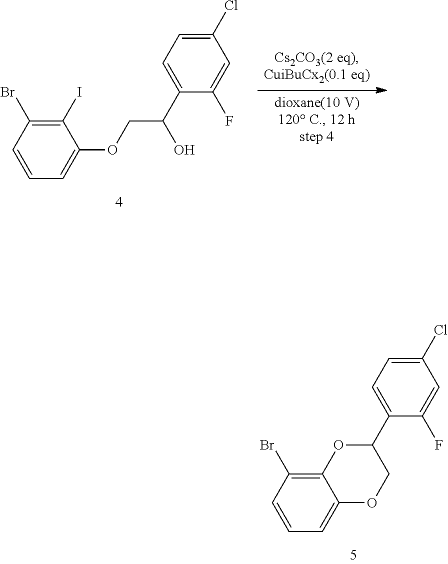 [SMILES: OC(COc1cccc(Br)c1I)c1ccc(Cl)cc1F ; "Cs2CO3(2 eq),CuiBuCx2(0.1 eq)" ; "dioxane(10 V)120° C., 12 hstep 4" ; Fc1cc(Cl)ccc1C1COc2cccc(Br)c2O1]

To a solution of 4 (1.5 g, 3.18 mmol, 1 eq) in dioxane (15 mL) was added Cs₂CO₃ (2.07 g, 6.36 mmol, 2 eq) and CuiBuCx₂ (126.62 mg, 318.14 μmol, 0.1 eq). The mixture was stirred at 120° C. for 12 hr. TLC (Petroleum ether/Ethyl acetate=5:1) indicated 4 was completely and one new spot formed. The reaction was clean according to TLC. The reaction mixture was added to H₂O 20 mL at 20° C. The mixture was extracted by Ethyl acetate (20 mL*3). Then organic phase was combined and washed by brine (10 mL), dried with Na₂SO₄, filtered. The organic phase was concentrated under reduced pressure at 30° C. to give a residue. The residue was purified by column chromatography (SiO₂, Petroleum ether/Ethyl acetate=100/0 to 100/0). 5 (865 mg, 2.39 mmol, 75.18% yield, 95% purity) was obtained as a white solid.

¹H NMR (400 MHz, DMSO-d₆) δ=7.64-7.55 (m, 2H), 7.46 (dd, J=2.0, 8.4 Hz, 1H), 7.23 (dd, J=1.2, 8.0 Hz, 1H), 7.03-7.00 (m, 1H), 6.88 (t, J=8.0 Hz, 1H), 5.62 (dd, J=2.4, 8.0 Hz, 1H), 4.55 (dd, J=2.4, 11.6 Hz, 1H), 4.23 (dd, J=8.0, 11.6 Hz, 1H)

### General Procedure for Preparation of Intermediate 6:

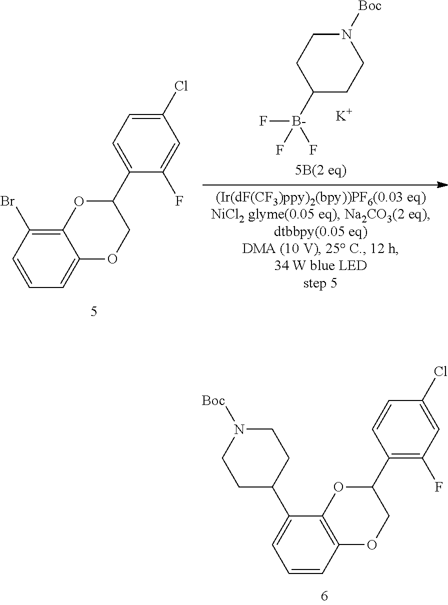

To a solution of 5 (500 mg, 1.46 mmol, 1 eq) and 5B (847.44 mg, 2.91 mmol, 2 eq) in DMA (15 mL) was added NiCl₂ glyme (15.99 mg, 72.76 μmol, 0.05 eq) and Na₂CO₃ (308.49 mg, 2.91 mmol, 2 eq) and dtbbpy (19.53 mg, 72.76 μmol, 0.05 eq) and (Ir(dF(CF₃)ppy)₂(bpy))PF₆ (44.08 mg, 43.66 μmol, 0.03 eq). The mixture was stirred at 25° C. for 12 hr. The mixture was monitoring by LCMS showed 5 was consumed completely and one main peak with desired m/z was detected. TLC (Petroleum ether/Ethyl acetate=5:1) indicated 5 was completely and one new spot formed. The reaction was clean according to TLC. The reaction mixture was added to H₂O 30 mL at 20° C. The mixture was extracted by Ethyl acetate (30 mL*3). Then organic phase was combined and washed by brine (30 mL), dried with Na₂SO₄, filtered. The organic phase was concentrated under reduced pressure at 30° C. to give a residue. The residue was purified by column chromatography (SiO₂, Petroleum ether/Ethyl acetate=100/1 to 50/1). 6 (0.94 g, 1.99 mmol, 45.66% yield, 95% purity) was obtained as a white solid.

LCMS: RT=1.810 min, MS cal.: 447.16, 449.16, [M−55]⁺=391.9

¹H NMR (400 MHz, DMSO-d₆) δ=7.61-7.48 (m, 2H), 7.47-7.34 (m, 1H), 6.87-6.75 (m, 3H), 5.47 (br d, J=6.0 Hz, 1H), 4.44 (dd, J=2.0, 11.6 Hz, 1H), 4.16-3.98 (m, 3H), 2.99 (br t, J=11.6 Hz, 1H), 2.76 (br s, 2H), 1.80-1.65 (m, 2H), 1.61-1.44 (m, 2H), 1.38 (s, 9H)

### General Procedure for Preparation of Intermediate 6A& Intermediate 6B:

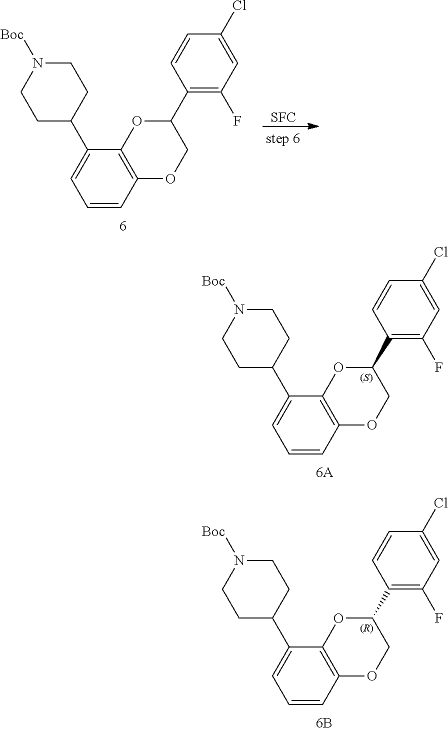

The residue was further separated by SFC (column: DAICEL CHIRALCEL OJ (250 mm*50 mm, 10 um); mobile phase: [CO₂-MeOH]; B %: 25%, isocratic elution mode). 6A (380 mg, 848.35 μmol, 42.22% yield) and 6B (400 mg, 848.35 μmol, 44.44% yield) was obtained as a white solid.

¹H NMR (400 MHz, DMSO-d₆) (6A) δ=7.62-7.54 (m, 2H), 7.44 (dd, J=1.6, 8.4 Hz, 1H), 6.90-6.81 (m, 3H), 5.50 (dd, J=2.0, 7.6 Hz, 1H), 4.47 (dd, J=2.4, 11.2 Hz, 1H), 4.14 (dd, J=8.0, 11.6 Hz, 1H), 4.10-4.03 (m, 2H), 3.07-2.97 (m, 1H), 2.93-2.67 (m, 2H), 1.84-1.68 (m, 2H), 1.64-1.44 (m, 3H), 1.41 (s, 9H)

¹H NMR (400 MHz, DMSO-d₆) (6B) δ=7.57 (dd, J=1.6, 10.4 Hz, 1H), 7.52 (t, J=8.4 Hz, 1H), 7.40 (dd, J=1.6, 8.4 Hz, 1H), 6.86-6.77 (m, 3H), 5.47 (dd, J=2.0, 7.6 Hz, 1H), 4.44 (dd, J=2.4, 11.6 Hz, 1H), 4.11 (dd, J=8.0, 11.6 Hz, 1H), 4.07-3.96 (m, 2H), 3.05-2.93 (m, 1H), 2.88-2.64 (m, 2H), 1.80-1.65 (m, 2H), 1.61-1.43 (m, 2H), 1.38 (s, 9H)

### General Procedure for Preparation of Intermediate 7A:

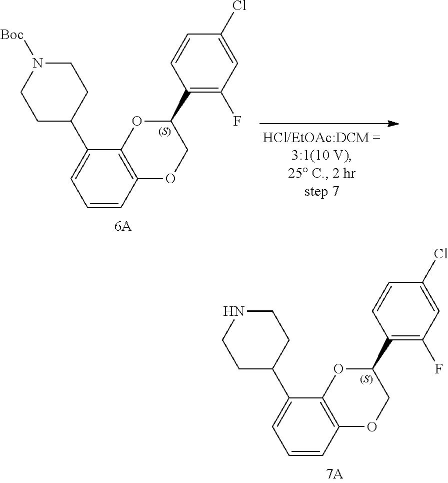

To a solution of 6A (340 mg, 759.05 μmol, 1 eq) in DCM (3 mL) was added HCl/EtOAc (9 mL). The mixture was stirred at 25° C. for 2 hr. LC-MS showed 6A was consumed completely and desired mass was detected. The organic phase was concentrated under reduced pressure at 40° C. to give a residue. 7A (290 mg, 754.68 μmol, 99.42% yield, HCl) was obtained as an orange oil.

LCMS: RT=1.185 min, MS cal.: 347.1, [M+H]⁺=348.1

¹H NMR (400 MHz, CHCl3-d) δ=7.41-7.33 (m, 1H), 7.26-7.22 (m, 1H), 7.21-7.16 (m, 1H), 6.95-6.85 (m, 2H), 6.82 (br d, J=7.2 Hz, 1H), 5.47-5.38 (m, 1H), 4.41 (dd, J=2.0, 11.3 Hz, 1H), 4.02 (dd, J=8.4, 11.2 Hz, 1H), 3.63 (br s, 2H), 3.21 (br d, J=1.6 Hz, 1H), 3.07 (br s, 2H), 2.22-1.98 (m, 5H)

### General Procedure for Preparation of Intermediate 9:

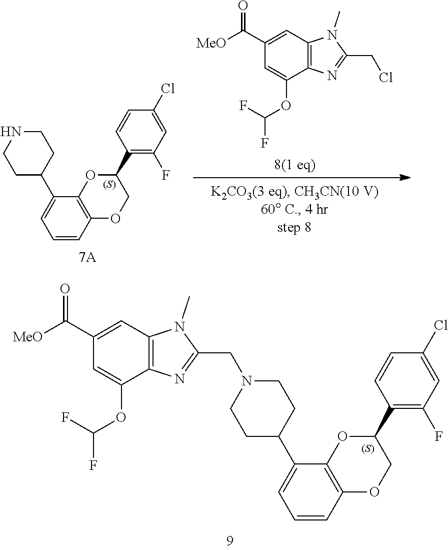

To a solution of 8 (86.49 mg, 283.89 μmol, 1 eq), 7A (120 mg, 312.28 μmol, 1.1 eq, HCl) in CH₃CN (2 mL) was added K₂CO₃ (117.71 mg, 851.67 μmol, 3 eq). The mixture was stirred at 60° C. for 4 hr. LC-MS showed 7A was consumed completely and desired mass was detected. The reaction mixture was added with aq. Na₂CO₃ to adjust pH=8-9. The residue was diluted with H₂O (10 mL) and extracted with EtOAc (15 mL). The combined organic layers were washed with brine (10 mL), dried over Na₂SO₄, filtered and concentrated under reduced pressure to give a residue. The residue was purified by column chromatography (SiO₂, Petroleum ether/Ethyl acetate=3/1 to 1/1). 9 (83 mg, 134.73 μmol, 47.46% yield) was obtained as a brown solid.

LCMS: RT=1.745 min, MS cal.: 615.16, [M+H]⁺=616.1

¹H NMR (400 MHz, DMSO-d₆) δ=8.10 (d, J=1.2 Hz, 1H), 7.64 (t, J=74.4 Hz, 1H), 7.57 (dd, J=2.0, 10.4 Hz, 1H), 7.55-7.48 (m, 2H), 7.42 (dd, J=2.0, 8.4 Hz, 1H), 6.84-6.80 (m, 1H), 6.77 (d, J=2.8 Hz, 1H), 6.84-6.75 (m, 1H), 5.46 (dd, J=2.0, 8.0 Hz, 1H), 4.44 (dd, J=2.4, 11.2 Hz, 1H), 4.10 (dd, J=8.0, 11.2 Hz, 1H), 3.94 (s, 3H), 3.90 (s, 3H), 3.83 (s, 2H), 2.93 (br d, J=11.2 Hz, 2H), 2.89-2.78 (m, 1H), 2.24-2.12 (m, 2H), 1.75-1.54 (m, 3H)

### General Procedure for Preparation of Compound 334 (10A):

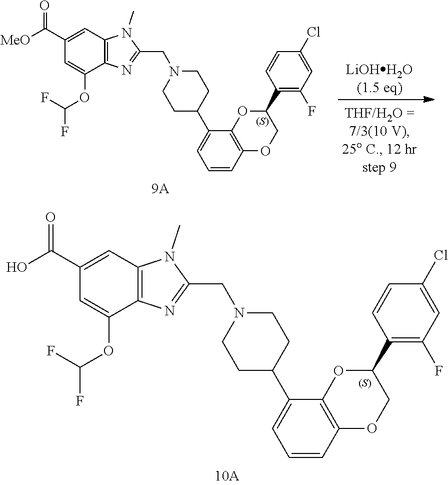

To a solution of 9 (100 mg, 162.33 μmol, 1 eq) in THF (0.7 mL) was added LiOH·H₂O (10.22 mg, 243.50 μmol, 1.5 eq) in H₂O (0.3 mL). The mixture was stirred at 25° C. for 12 hr. LC-MS showed 9 was consumed completely and desired mass was detected. The reaction mixture was purified directly. The residue was purified by prep-HPLC (neutral condition; column: Waters Xbridge Prep OBD C18 150*40 mm*10 um; mobile phase: [H₂O (10 mM NH₄HCO₃)-ACN]; gradient: 20%-70% B over 8.0 min). (S)-2-((4-(3-(4-Chloro-2-fluorophenyl)-2,3-dihydrobenzo[b][1,4]dioxin-5-yl)piperidin-1-yl)methyl)-4-(difluoromethoxy)-1-methyl-1H-benzo[d]imidazole-6-carboxylic acid (29.97 mg, 49.78 μmol, 30.67% yield) was obtained as a white solid.

LCMS: RT=1.251 min, MS cal.: 601.16, [M+H]⁺=602.2

LCMS: RT=2.862 min, MS cal.: 601.16, [M+H]⁺=602.2

HPLC: RT=12.600 min, purity: 100.0%

¹H NMR (400 MHz, DMSO-d₆) δ=8.06 (s, 1H), 7.62 (t, J=74.4 Hz, 1H), 7.57 (dd, J=1.6, 10.4 Hz, 1H), 7.55-7.49 (m, 2H), 7.45-7.40 (m, 1H), 6.86-6.75 (m, 3H), 5.46 (dd, J=1.6, 7.6 Hz, 1H), 4.44 (dd, J=2.0, 11.2 Hz, 1H), 4.10 (dd, J=8.0, 11.6 Hz, 1H), 3.93 (s, 3H), 3.83 (s, 2H), 2.92 (br d, J=10.8 Hz, 2H), 2.89-2.78 (m, 1H), 2.24-2.09 (m, 2H), 1.82-1.56 (m, 4H)

### General Procedure for Preparation of Intermediate 7B:

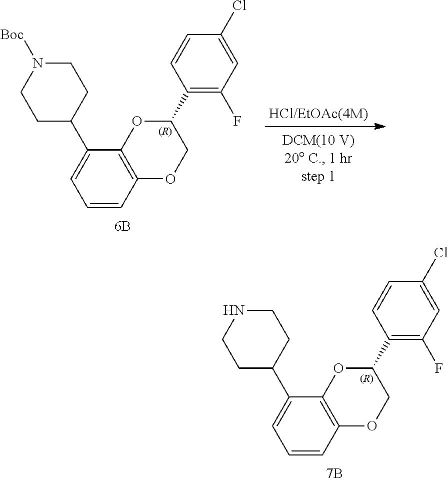

A mixture of 6B (360 mg, 803.70 μmol, 1 eq) in DCM (1 mL) and HCl/EtOAc (2.6 mL) was degassed and purged with N₂ for 3 times, and then the mixture was stirred at 20° C. for 1 hr under N₂ atmosphere. LCMS (Product, Rt=1.153 min) showed 95% of 6B was detected. The reaction mixture was concentrated under reduced pressure to give a residue. Without purification. 7B (300 mg, crude) was obtained as a white solid.

LCMS: RT=1.217 min, MS cal.: 347.1/349.1, [M+1]⁺=347.9/349.9

### General Procedure for Preparation of Intermediate 9B:

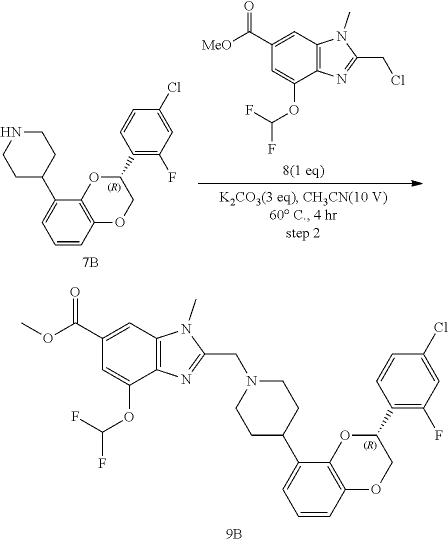

A mixture of 7B (70.00 mg, 182.16 μmol, 1.1 eq, HCl), 8 (50.46 mg, 165.60 μmol, 1 eq), K₂CO₃ (68.66 mg, 496.81 μmol, 3 eq) in CH₃CN (0.7 mL) was degassed and purged with N₂ for 3 times, and then the mixture was stirred at 60° C. for 4 hr under N₂ atmosphere. LCMS (product, RT=1.786 min) showed 91% of 7B was detected. The mixture was diluted with H₂O (10 ml) and then extracted with EtOAc (20 mL*3). The combined organic layers were washed with brine (20 mL), dried over Na₂SO₄, filtered, and concentrated under reduced pressure to give a residue. The crude product on notebook page ET57960-1005 was combined to ET57960-1008 for further purification. The residue was purified by column chromatography (SiO₂, Petroleum ether/Ethyl acetate=3/1 to 1/1). Compound 9B (120 mg, crude) was obtained as a white solid.

LCMS: RT=1.786 min, MS cal.: 615.17/616.18, [M+1]⁺=616/618.1

LCMS: RT=1.773 min, MS cal.: 615.17/616.18, [M+1]⁺=616.1/618.0

### General Procedure for Preparation of 335 (10B):

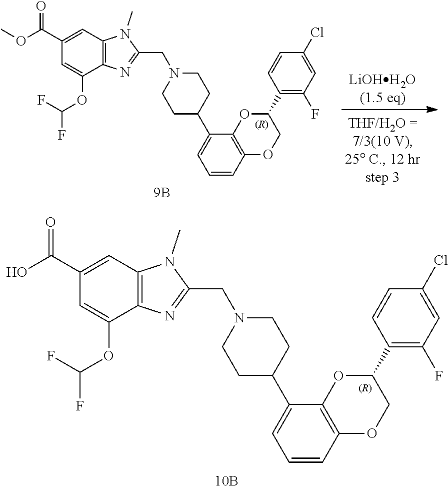

A mixture of 9B (110 mg, 178.56 μmol, 1 eq), LiOH·H₂O (11.24 mg, 267.85 μmol, 1.5 eq) in THF (0.77 mL) and H₂O (0.33 mL) was degassed and purged with N₂ for 3 times, and then the mixture was stirred at 25° C. for 12 hr under N₂ atmosphere. LCMS (product, RT=1.195 min) showed 52% of 9B was detected. The reaction mixture was purified directly. The residue was purified by prep-HPLC (neutral condition column: Waters Xbridge Prep OBD C18 150*40 mm*10 um; mobile phase: [H₂O (10 mM NH₄HCO₃)-ACN]; gradient: 30%-80% B over 8.0 min). (R)-2-((4-(3-(4-Chloro-2-fluorophenyl)-2,3-dihydrobenzo[b][1,4]dioxin-5-yl)piperidin-1-yl)methyl)-4-(difluoromethoxy)-1-methyl-1H-benzo[d]imidazole-6-carboxylic acid (29.04 mg, 48.24 μmol, 27.02% yield) was obtained as a white solid.

LCMS: RT=2.858 min, MS cal.: 614.2/615.2, [M+H]⁺=602.2/604.2

HPLC: RT=12.588 min, purity: 99.87%

1H NMR (400 MHz, DMSO-d6) δ=8.05 (s, 1H), 7.62 (t, J=74.4 Hz, 1H), 7.59 (d, J=2.0 Hz, 1H), 7.54-7.50 (m, 2H), 7.45-7.40 (m, 1H), 6.84-6.76 (m, 3H), 5.46 (dd, J=2.4, 7.7 Hz, 1H), 4.44 (dd, J=2.4, 11.2 Hz, 1H), 4.10 (dd, J=8.0, 11.4 Hz, 1H), 3.92 (s, 3H), 3.82 (s, 2H), 2.92 (br d, J=10.8 Hz, 2H), 2.88-2.77 (m, 1H), 2.24-2.11 (m, 2H), 1.85-1.58 (m, 4H)

### Example A71—Preparation of Compound 301 and Compound 336. 2-((4-(6-((4-Cyano-2-fluorobenzyl)oxy)pyridin-2-yl)piperidin-1-yl)methyl)-5-fluoro-4-methoxy-1-methyl-1H-benzo[d]imidazole-6-carboxylic acid (Compound 301) (3) & 2-((4-(6-((4-Cyano-2-fluorobenzyl)oxy)pyridin-2-yl)piperidin-1-yl)methyl)-6-fluoro-7-methoxy-1-methyl-1H-benzo[d]imidazole-5-carboxylic acid (Compound 336) (5) Scheme 1

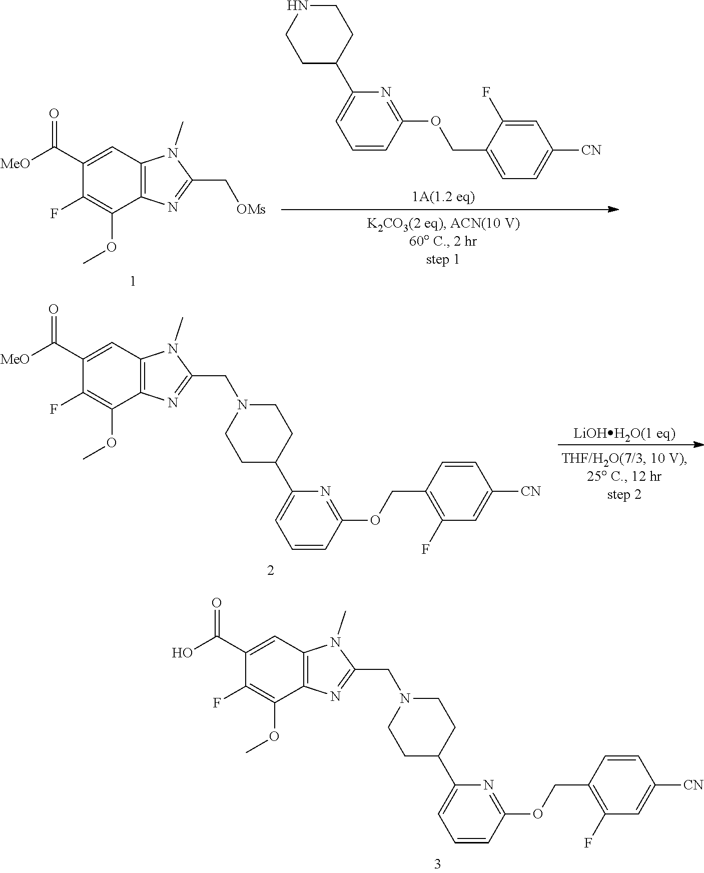 [SMILES: COC(=O)c1cc2c(nc(COS(C)(=O)=O)n2C)c(OC)c1F ; N#Cc1ccc(COc2cccc(C3CCNCC3)n2)c(F)c1 ; COC(=O)c1cc2c(nc(CN3CCC(c4cccc(OCc5ccc(C#N)cc5F)n4)CC3)n2C)c(OC)c1F ; COc1c(F)c(C(=O)O)cc2c1nc(CN1CCC(c3cccc(OCc4ccc(C#N)cc4F)n3)CC1)n2C]

### General Procedure for Preparation of Intermediate 2:

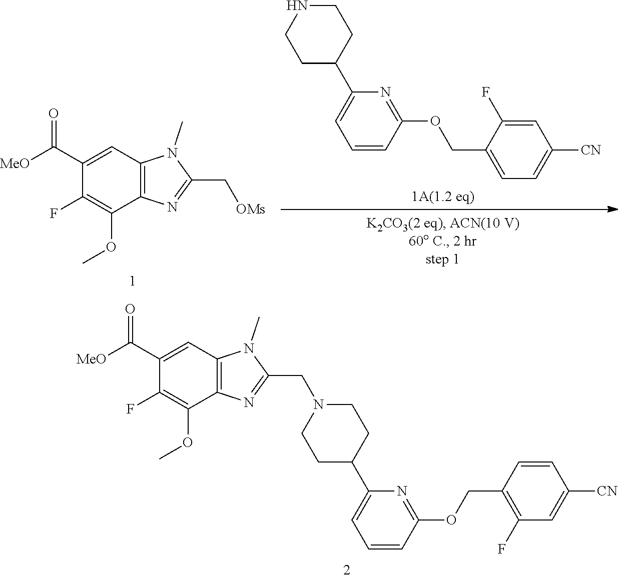 [SMILES: COC(=O)c1cc2c(nc(COS(C)(=O)=O)n2C)c(OC)c1F ; N#Cc1ccc(COc2cccc(C3CCNCC3)n2)c(F)c1 ; COC(=O)c1cc2c(nc(CN3CCC(c4cccc(OCc5ccc(C#N)cc5F)n4)CC3)n2C)c(OC)c1F]

A mixture of 1 (100 mg, 288.74 μmol, 1 eq), 1A (120.51 mg, 346.49 μmol, 1.2 eq, HCl), K₂CO₃ (119.72 mg, 866.22 μmol, 3 eq) in ACN (2 mL) was degassed and purged with N₂ for 3 times, and then the mixture was stirred at 60° C. for 2 hr under N₂ atmosphere. LC-MS showed 1 was consumed completely and one main peak with desired mass was detected. The mixture was filtered and concentrated in vacuum. The residue was purified by prep-TLC (SiO₂, PE:EA=1:2, R (=0.50). 2 (135 mg, 228.59 μmol, 79.17% yield) was obtained as a yellow oil.

LCMS: RT=0.444 min, MS cal.: 561.2, [M+H]⁺=562.1

HPLC: product: RT=2.609 min, purity: 95.09%

¹H NMR (400 MHz, DMSO-d6) ¿=7.87 (d, J=10.0 Hz, 1H), 7.70 (dd, J=5.2, 12.4 Hz, 3H), 7.64 (t, J=7.6 Hz, 1H), 6.87 (d, J=7.2 Hz, 1H), 6.71 (d, J=8.0 Hz, 1H), 5.45 (s, 2H), 4.26 (s, 3H), 3.90 (s, 3H), 3.87 (s, 3H), 3.82 (s, 2H), 2.91 (br d, J=11.2 Hz, 2H), 2.61-2.53 (m, 1H), 2.20 (br t, J=10.0 Hz, 2H), 1.79-1.60 (m, 4H)

### General Procedure for Preparation of Compound 301 (3):

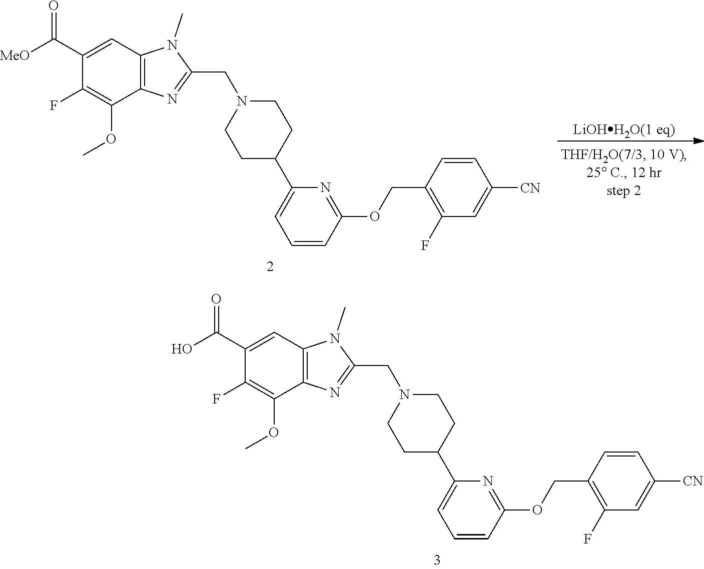 [SMILES: COC(=O)c1cc2c(nc(CN3CCC(c4cccc(OCc5ccc(C#N)cc5F)n4)CC3)n2C)c(OC)c1F ; COc1c(F)c(C(=O)O)cc2c1nc(CN1CCC(c3cccc(OCc4ccc(C#N)cc4F)n3)CC1)n2C]

To a solution of 2 (120 mg, 213.68 μmol, 1 eq) in THF (1.4 mL) and H₂O (0.6 mL) was added LiOH·H₂O (8.97 mg, 213.68 μmol, 1 eq). The mixture was stirred at 25° C. for 15 hr. LC-MS showed 2 was consumed completely and one main peak with desired mass was detected. The mixture was filtered and concentrated in vacuum. The residue was purified by prep-HPLC (column: Waters Xbridge Prep OBD C₁₈ 150*40 mm*10 um; mobile phase: [H₂O (10 mM NH₄HCO₃)-ACN]; gradient: 15%-55% B over 8.0 min). 2-((4-(6-((4-Cyano-2-fluorobenzyl)oxy)pyridin-2-yl)piperidin-1-yl)methyl)-5-fluoro-4-methoxy-1-methyl-1H-benzo[d]imidazole-6-carboxylic acid (28.99 mg, 52.82 μmol, 24.72% yield) was obtained as a white solid.

LCMS: RT=2.524 min, MS cal.: 547.2, [M+H]⁺=548.2

HPLC: product: RT=10.078 min, purity: 99.77%

¹H NMR (400 MHz, MeOH-d4) δ=7.66 (t, J=8.0 Hz, 1H), 7.63-7.50 (m, 4H), 6.84 (d, J=7.6 Hz, 1H), 6.68 (d, J=8.0 Hz, 1H), 5.50 (s, 2H), 4.17 (s, 3H), 3.99 (s, 2H), 3.93 (s, 3H), 3.16 (br d, J=11.6 Hz, 2H), 2.68 (br t, J=8.0 Hz, 1H), 2.52-2.42 (m, 2H), 1.93-1.83 (m, 4H)

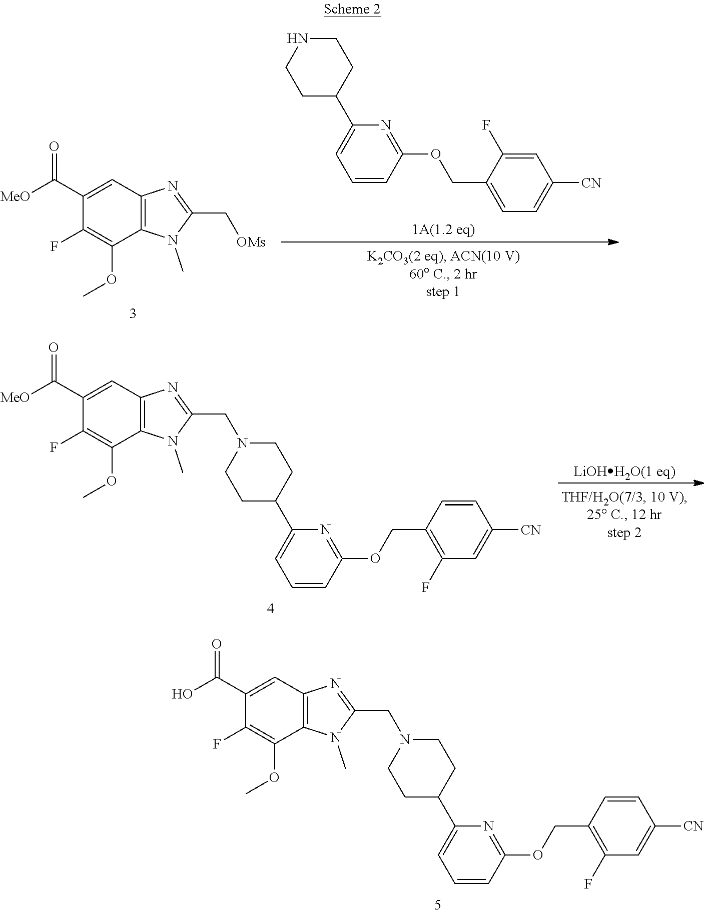 [SMILES: COC(=O)c1cc2nc(COS(C)(=O)=O)n(C)c2c(OC)c1F ; N#Cc1ccc(COc2cccc(C3CCNCC3)n2)c(F)c1 ; COC(=O)c1cc2nc(CN3CCC(c4cccc(OCc5ccc(C#N)cc5F)n4)CC3)n(C)c2c(OC)c1F ; COc1c(F)c(C(=O)O)cc2nc(CN3CCC(c4cccc(OCc5ccc(C#N)cc5F)n4)CC3)n(C)c12]

### General Procedure for Preparation of Intermediate 4:

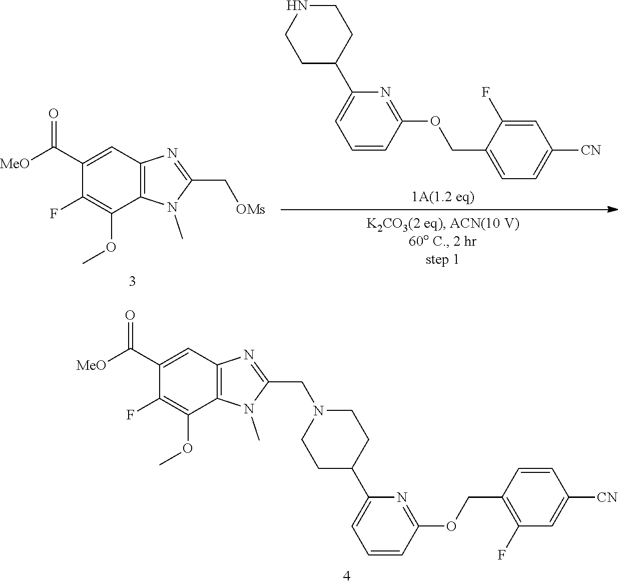 [SMILES: COC(=O)c1cc2nc(COS(C)(=O)=O)n(C)c2c(OC)c1F ; N#Cc1ccc(COc2cccc(C3CCNCC3)n2)c(F)c1 ; COC(=O)c1cc2nc(CN3CCC(c4cccc(OCc5ccc(C#N)cc5F)n4)CC3)n(C)c2c(OC)c1F]

A mixture of methyl 3 (70 mg, 202.12 μmol, 1 eq), 1A (84.36 mg, 242.54 μmol, 1.2 eq, HCl), K₂CO₃ (111.74 mg, 808.48 μmol, 4 eq) in ACN (1 mL) was degassed and purged with N₂ for 3 times, and then the mixture was stirred at 60° C. for 2 hr under N₂ atmosphere. LC-MS showed 3 was consumed completely and one main peak with desired mass was detected. The mixture was filtered and concentrated in vacuum. The residue was purified by prep-TLC (SiO₂, Petroleum ether:Ethyl acetate=1:2, R_{f}=0.40). 4 (105 mg, 186.97 μmol, 92.51% yield) was obtained as a yellow oil.

LCMS: RT=0.452 min, MS cal.: 561.2 [M+H]⁺=562.3

¹H NMR (400 MHz, DMSO-d6) δ=7.87 (d, J=10.4 Hz, 1H), 7.82 (d, J=5.2 Hz, 1H), 7.69 (d, J=3.6 Hz, 2H), 7.66-7.61 (m, 1H), 6.87 (d, J=7.6 Hz, 1H), 6.71 (d, J=8.0 Hz, 1H), 5.44 (s, 2H), 4.05 (s, 3H), 4.02 (s, 3H), 3.85 (s, 3H), 3.77 (s, 2H), 2.91 (br d, J=11.2 Hz, 2H), 2.62-2.52 (m, 1H), 2.22-2.13 (m, 2H), 1.79-1.59 (m, 4H)

### General Procedure for Preparation of Compound 336 (5):

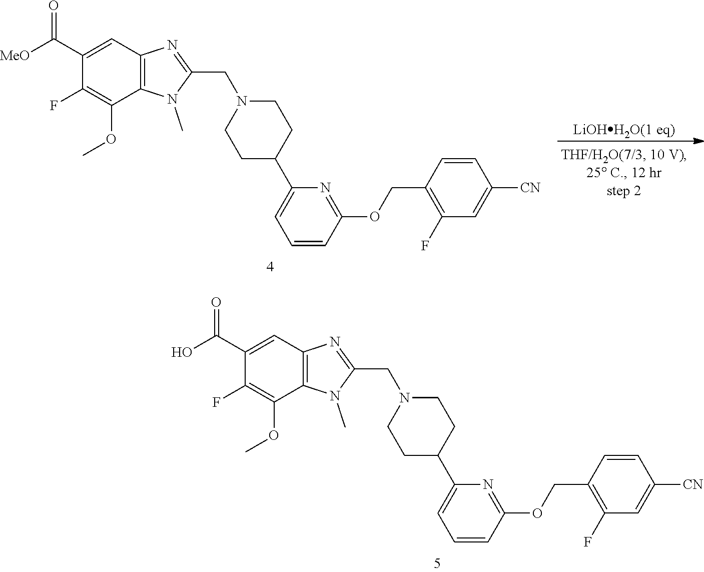 [SMILES: COC(=O)c1cc2nc(CN3CCC(c4cccc(OCc5ccc(C#N)cc5F)n4)CC3)n(C)c2c(OC)c1F ; COc1c(F)c(C(=O)O)cc2nc(CN3CCC(c4cccc(OCc5ccc(C#N)cc5F)n4)CC3)n(C)c12]

To a solution of 4 (100 mg, 178.07 μmol, 1 eq) in THF (0.7 mL) and H₂O (0.3 mL) was added LiOH·H₂O (7.47 mg, 178.07 μmol, 1 eq). The mixture was stirred at 25° C. for 15 hr. LC-MS showed 4 was consumed completely and one main peak with desired mass was detected. The mixture was concentrated in vacuum. The residue was purified by prep-HPLC (column: Waters Xbridge Prep OBD C₁₈ 150*40 mm*10 um; mobile phase: [H₂O (10 mM NH₄HCO₃)-ACN]; gradient: 15%-65% B over 8.0 min). 2-((4-(6-((4-Cyano-2-fluorobenzyl)oxy)pyridin-2-yl)piperidin-1-yl)methyl)-6-fluoro-7-methoxy-1-methyl-1H-benzo[d]imidazole-5-carboxylic acid (28.41 mg, 51.77 μmol, 29.07% yield) was obtained as a white solid.

LCMS: RT=2.611 min, MS cal.: 547.2, [M+H]⁺=548.2

HPLC: product: RT=9.817 min, purity: 99.77%

¹H NMR (400 MHz, MeOH-d4) δ=7.80 (d, J=5.2 Hz, 1H), 7.66 (t, J=7.6 Hz, 1H), 7.62-7.50 (m, 3H), 6.84 (d, J=7.2 Hz, 1H), 6.68 (d, J=7.6 Hz, 1H), 5.50 (s, 2H), 4.14 (s, 3H), 4.08 (d, J=0.8 Hz, 3H), 3.89 (s, 2H), 3.09 (br d, J=11.2 Hz, 2H), 2.71-2.60 (m, 1H), 2.43-2.32 (m, 2H), 1.90-1.81 (m, 4H)

### Example A72—Preparation of Compound 337 and Compound 338. 2-((4-(6-((4-Chloro-2-fluorobenzyl)oxy)pyridin-2-yl)piperidin-1-yl)methyl)-5-fluoro-4-methoxy-1-methyl-1H-benzo[d]imidazole-6-carboxylic acid (Compound 337) (10) and 2-((4-(6-((4-chloro-2-fluorobenzyl)oxy)pyridin-2-yl)piperidin-1-yl)methyl)-6-fluoro-7-methoxy-1-methyl-1H-benzo[d]imidazole-5-carboxylic acid (Compound 338) (10A)

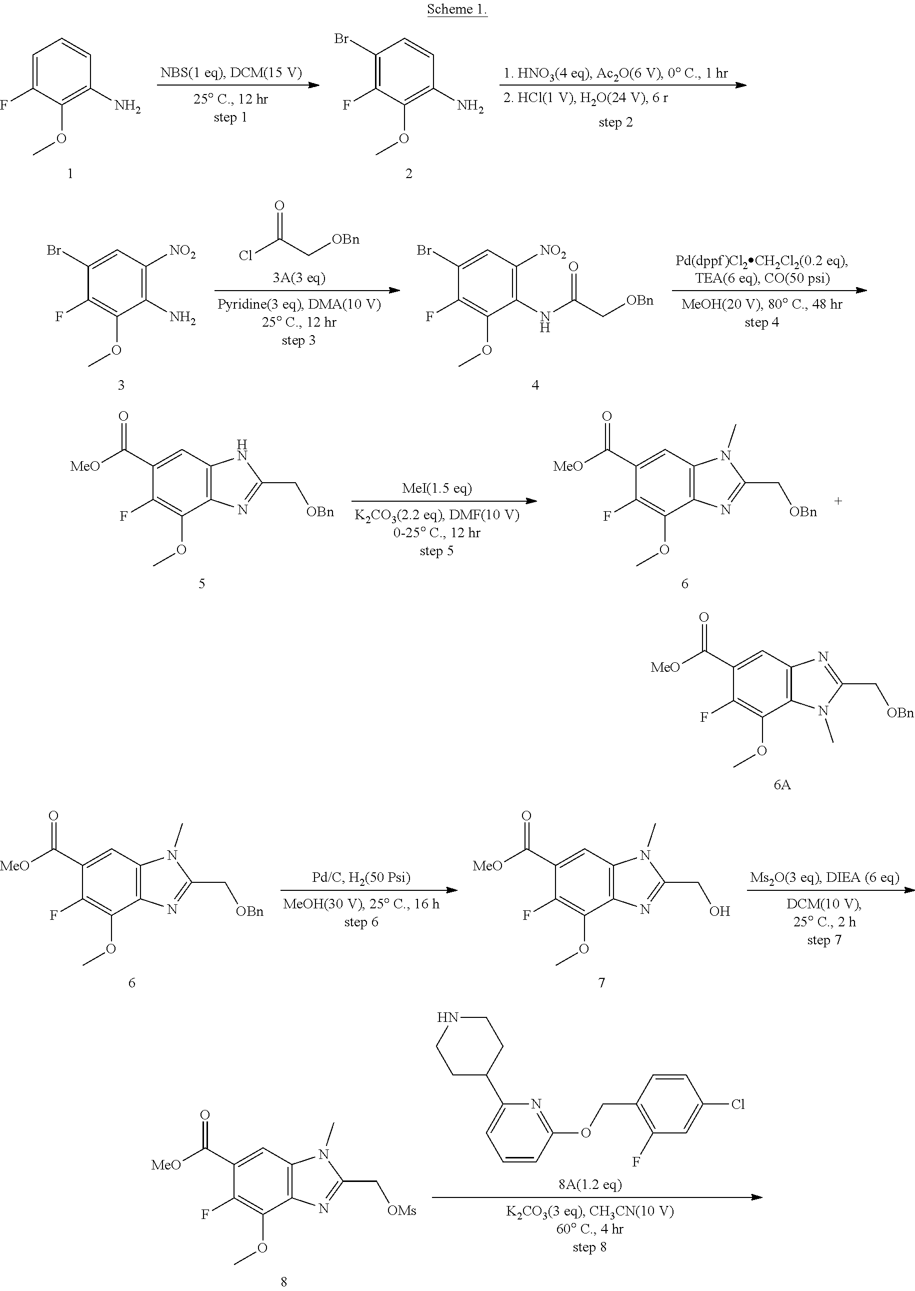

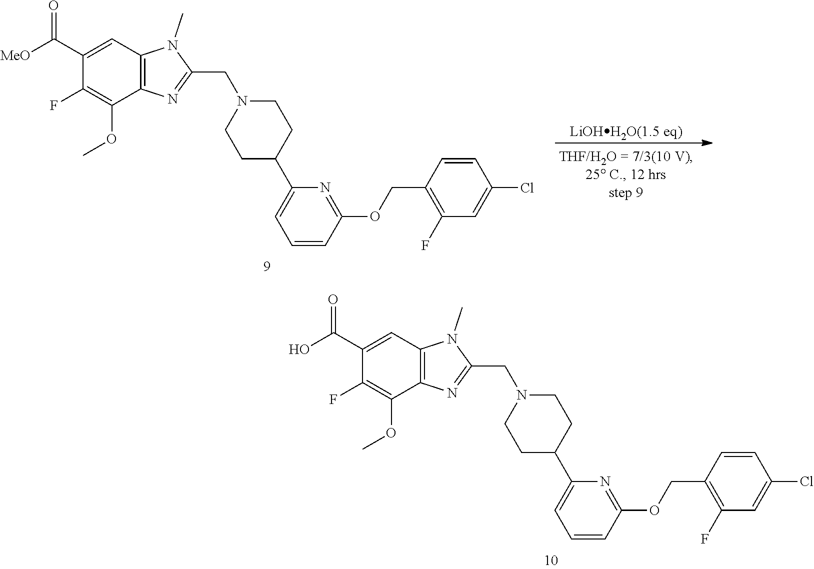 [SMILES: COC(=O)c1cc2c(nc(CN3CCC(c4cccc(OCc5ccc(Cl)cc5F)n4)CC3)n2C)c(OC)c1F ; COc1c(F)c(C(=O)O)cc2c1nc(CN1CCC(c3cccc(OCc4ccc(Cl)cc4F)n3)CC1)n2C]

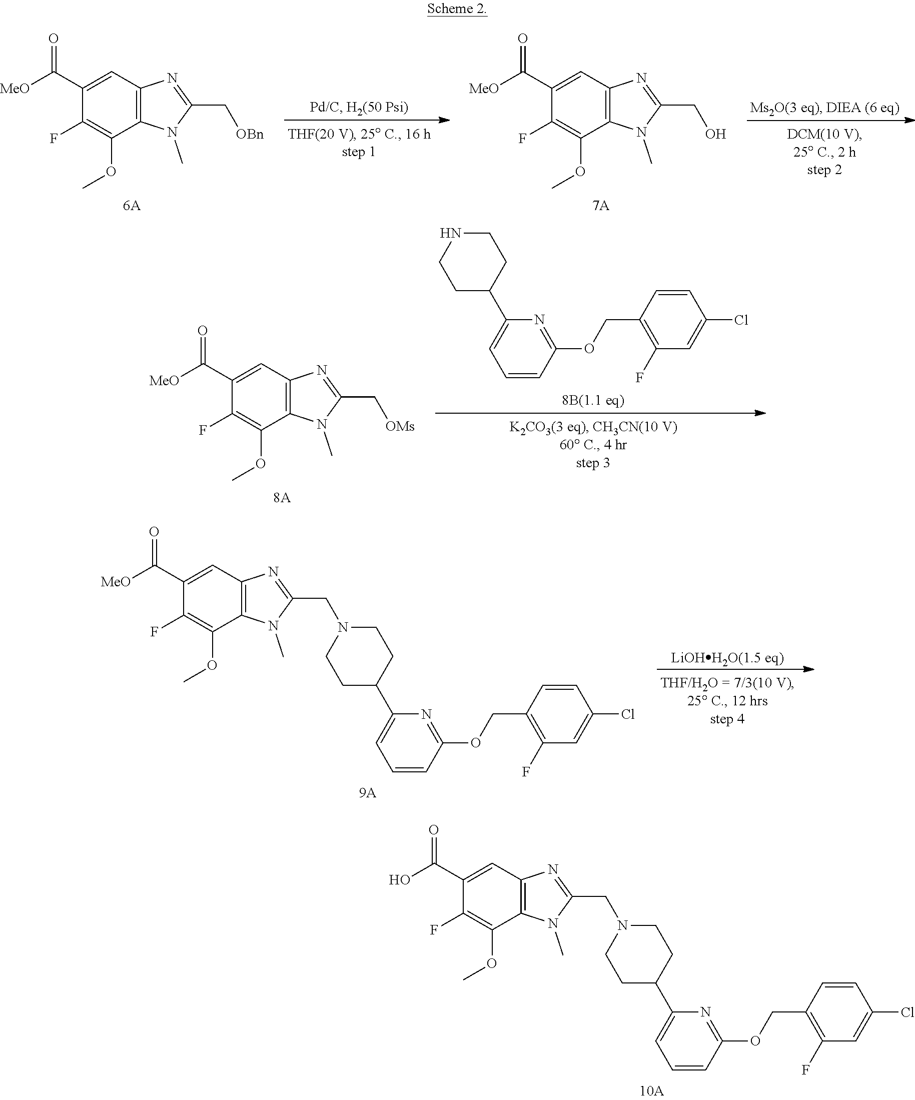 [SMILES: COC(=O)c1cc2nc(COCc3ccccc3)n(C)c2c(OC)c1F ; COC(=O)c1cc2nc(CO)n(C)c2c(OC)c1F ; COC(=O)c1cc2nc(COS(C)(=O)=O)n(C)c2c(OC)c1F ; Fc1cc(Cl)ccc1COc1cccc(C2CCNCC2)n1 ; COC(=O)c1cc2nc(CN3CCC(c4cccc(OCc5ccc(Cl)cc5F)n4)CC3)n(C)c2c(OC)c1F ; COc1c(F)c(C(=O)O)cc2nc(CN3CCC(c4cccc(OCc5ccc(Cl)cc5F)n4)CC3)n(C)c12]

### General Procedure for Preparation of Scheme 1, Intermediate 2:

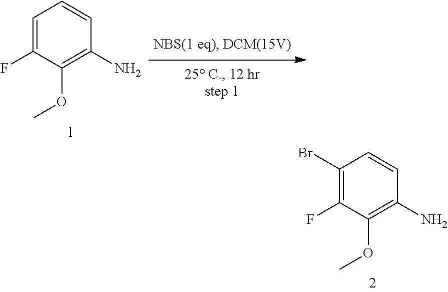

To a solution of 1 (8 g, 56.68 mmol, 1 eq) in DCM (120 mL) was added NBS (10.59 g, 59.51 mmol, 1.05 eq). The mixture was stirred at 25° C. for 15 hrs. The color changes to brown. TLC (SiO₂, Petroleum ether:Ethyl acetate=10:1, R_{f}=0.40) showed the reaction was completed. The residue was poured into water (500 mL) and the aqueous phase was extracted with dichloromethane (100 mL*3). The combined organic phase was washed with saturated brine (50 mL), dried with anhydrous Na₂SO₄, filtered, and concentrated in vacuum. The residue was purified by prep-HPLC (column: Waters Xbridge BEH C18 250* 70 mm*10 um; mobile phase: [H₂O (10 mM NH₄HCO₃)-ACN]; gradient: 30%-60% B over 20.0 min). 2 (6.9 g, 26.03 mmol, 45.92% yield) was obtained as a brown solid.

LCMS: RT=0.459 min, MS cal.: 221.0, [M+H]⁺=221.9

¹H NMR (400 MHz, DMSO-d6) δ=7.01 (dd, J=7.6, 8.8 Hz, 1H), 6.46 (dd, J=1.6, 8.8 Hz, 1H), 5.40 (s, 2H), 3.74 (s, 3H)

### General Procedure for Preparation of Scheme 1, Intermediate 3:

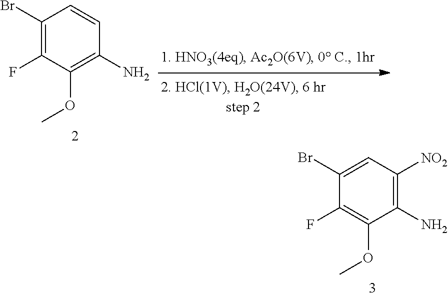

To a mixture of 2 (5 g, 22.72 mmol, 1 eq) in Ac₂O (37.5 mL) was added HNO₃ (5.73 g, 90.89 mmol, 4.09 mL, 4 eq) dropwise at 0° C. The mixture was stirred at 0° C. for 30 min. The mixture was added ice-H₂O (150 mL) and HCl (7.5 mL) and heated to 100° C. for 12 h. The color changes to yellow. TLC (SiO₂, Petroleum ether:Ethyl acetate=3:1, R_{f}=0.40) showed the reaction was completed. The reaction mixture was cooled to 0° C. and added NH₃·H₂O to pH=8-9 and extracted with EA 240 mL (80 mL*3). The combined organic layers were washed with Brine 200 mL, dried over Na₂SO₄, filtered and concentrated under reduced pressure to give a residue. The residue was purified by column chromatography (SiO₂, Petroleum ether:Ethyl acetate=1/0 to 0/1). 3 (2.95 g, 11.13 mmol, 48.98% yield) was obtained as a yellow solid.

¹H NMR (400 MHz, DMSO-d6) δ=8.03 (d, J=7.2 Hz, 1H), 7.47 (br s, 2H), 3.87 (d, J=1.2 Hz, 3H)

### General Procedure for Preparation of Scheme 1, Intermediate 4:

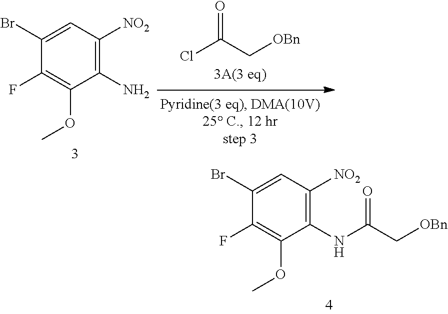 [SMILES: COc1c(N)c([N+](=O)[O-])cc(Br)c1F ; O=C(Cl)COCc1ccccc1 ; COc1c(F)c(Br)cc([N+](=O)[O-])c1NC(=O)COCc1ccccc1]

To a mixture of 3 (2.95 g, 11.13 mmol, 1 eq) and Pyridine (2.64 g, 33.39 mmol, 2.70 mL, 3 eq) in DMA (30 mL) was added 3A (6.16 g, 33.39 mmol, 5.18 mL, 3 eq) at 0° C. The mixture was stirred at 20° C. for 12 h. The color changes to yellow. LCMS showed the reaction was completed. The reaction mixture was quenched by addition water 50 mL, and then extracted with EA 60 mL (20 mL*3). The combined organic layers were washed with brine 60 mL*3, dried over Na₂SO₄, filtered and concentrated under reduced pressure to give a residue. The residue was purified by prep-HPLC (column: Welch Xtimate C18 250* 100 mm #10 um; mobile phase: [H₂O (10 mM NH₄HCO₃)-ACN]; gradient: 45%-75% B over 18.0 min). 4 (2.72 g, 6.56 mmol, 58.92% yield) was obtained as a yellow solid.

LCMS: RT=1.394 min, MS cal.: 412.0, [M−H]⁺=410.8

¹H NMR (400 MHz, DMSO-d6) δ=10.00 (s, 1H), 8.14 (d, J=6.4 Hz, 1H), 7.43-7.36 (m, 4H), 7.36-7.30 (m, 1H), 4.61 (s, 2H), 4.16 (s, 2H), 3.93 (d, J=2.0 Hz, 3H)

### General Procedure for Preparation of Scheme 1, Intermediate 5:

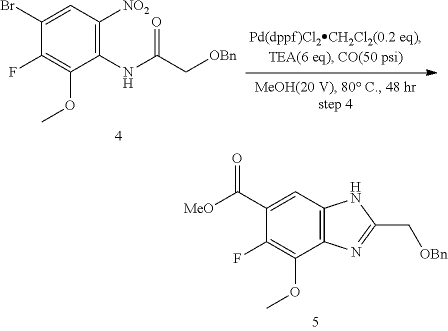 [SMILES: COc1c(F)c(Br)cc([N+](=O)[O-])c1NC(=O)COCc1ccccc1 ; COC(=O)c1cc2[nH]c(COCc3ccccc3)nc2c(OC)c1F]

MeOH (100 mL) and TEA (7.93 g, 78.41 mmol, 10.91 mL, 6 eq) was charged to the 250 mL Hydrogenated bottle, then 4 (5.4 g, 13.07 mmol, 1 eq) and Pd(dppf)Cl₂·CH₂Cl₂ (2.13 g, 2.61 mmol, 0.2 eq) was added at 25° C. After the addition, the mixture was degassed and purged with CO for 3 times. The mixture was stirred at 80° C. (50 psi) for 72 hrs. The color changes to brown. TLC (SiO₂, PE:EA=1:1, R_{f}=0.50) showed the reaction was completed. The mixture was filtered and concentrated in vacuum. The residue was purified by column chromatography (SiO₂, Petroleum ether/Ethyl acetate=2/1 to 1/1). 5 (3.7 g, 10.66 mmol, 81.55% yield) was obtained as a yellow solid.

LCMS: RT=0.498 min, MS cal.: 344.1, [M+H]⁺=345.2

HPLC: RT=1.994 min, purity: 99.18%

¹H NMR (400 MHz, DMSO-d6) δ=13.10-12.85 (m, 1H), 7.67-7.55 (m, 1H), 7.43-7.35 (m, 4H), 7.34-7.28 (m, 1H), 4.78 (br s, 2H), 4.64 (s, 2H), 4.36-4.09 (m, 3H), 3.85 (s, 3H)

### General Procedure for Preparation of Scheme 1, Intermediate 6:

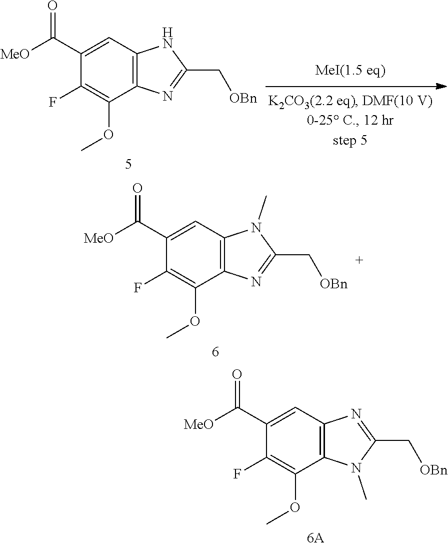 [SMILES: COC(=O)c1cc2[nH]c(COCc3ccccc3)nc2c(OC)c1F ; COC(=O)c1cc2c(nc(COCc3ccccc3)n2C)c(OC)c1F ; COC(=O)c1cc2nc(COCc3ccccc3)n(C)c2c(OC)c1F]

To a mixture of 5 (4.1 g, 11.91 mmol, 1 eq) and K₂CO₃ (3.62 g, 26.20 mmol, 2.2 eq) in DMF (41 mL) was added CH₃I (2.54 g, 17.86 mmol, 1.11 mL, 1.5 eq) at 0° C. The mixture was stirred at 25° C. for 1 hour. The color changes to yellow. TLC (SiO₂, PE:EA=1:1, R_{f}=0.50, 0.40) showed the reaction was completed. The reaction mixture was quenched by addition water 200 mL, and then extracted with DCM 300 mL (100 mL*3). The combined organic layers were washed with brine 300 mL, dried over Na₂SO₄, filtered, and concentrated under reduced pressure to give a residue. The residue was purified by column chromatography (SiO₂, Petroleum ether/Ethyl acetate=3/1 to 2/1). 6 (1.5 g, 4.19 mmol, 35.15% yield) was obtained as a white solid. 6A (1.2 g, 3.35 mmol, 28.12% yield) was obtained as a yellow solid.

¹H NMR (400 MHz, DMSO-d6) δ=7.72 (d, J=5.2 Hz, 1H), 7.36 (d, J=4.4 Hz, 4H), 7.33-7.27 (m, 1H), 4.82 (s, 2H), 4.60 (s, 2H), 4.28 (s, 3H), 3.87 (s, 3H), 3.84 (s, 3H)

¹H NMR (400 MHz, DMSO-d6) δ=7.86 (d, J=5.6 Hz, 1H), 7.36 (d, J=4.0 Hz, 4H), 7.33-7.28 (m, 1H), 4.79 (s, 2H), 4.58 (s, 2H), 4.01-3.97 (m, 6H), 3.86 (s, 3H)

### General Procedure for Preparation of Scheme 1, Intermediate 7:

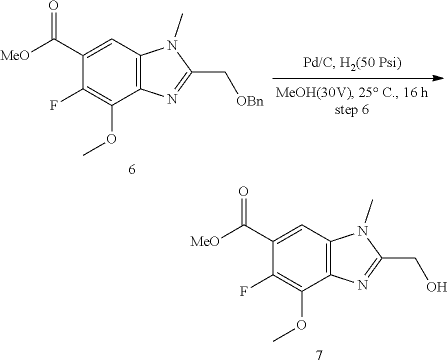 [SMILES: COC(=O)c1cc2c(nc(COCc3ccccc3)n2C)c(OC)c1F ; COC(=O)c1cc2c(nc(CO)n2C)c(OC)c1F]

To a solution of 6 (600 mg, 1.67 mmol, 1 eq) in MeOH (20 mL) was added Pd/C (178.18 mg, 167.43 μmol, 10% purity, 0.1 eq) under N₂ atmosphere. The suspension was degassed and purged with H₂ for 3 times. The mixture was stirred under H₂ (15 Psi) at 25° C. for 24 hours. The color changes to black. LCMS showed the reaction was completed. The mixture was filtered and concentrated in vacuum. The mixture was used for next step directly without purification. 7 (400 mg, 1.41 mmol, 84.18% yield) was obtained as a white solid.

LCMS: RT=0.287 min, MS cal.: 268.1, [M+H]⁺=269.2.

HPLC: RT=1.159 min, purity: 94.51%

¹H NMR (400 MHz, DMSO-d6) δ=7.70 (d, J=4.8 Hz, 1H), 5.71 (s, 1H), 4.73 (s, 2H), 4.26 (s, 3H), 3.86 (d, J=8.8 Hz, 6H)

### General Procedure for Preparation of Scheme 1, 8:

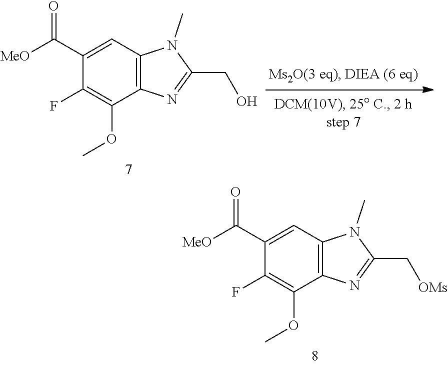 [SMILES: COC(=O)c1cc2c(nc(CO)n2C)c(OC)c1F ; COC(=O)c1cc2c(nc(COS(C)(=O)=O)n2C)c(OC)c1F]

To a mixture of 7 (400 mg, 1.49 mmol, 1 eq) and DIEA (1.16 g, 8.95 mmol, 1.56 mL, 6 eq) in DCM (8 mL) was added methylsulfonyl methanesulfonate (779.28 mg, 4.47 mmol, 3 eq). The mixture was stirred at 25° C. for 2 hours. The color changes to yellow. TLC (SiO₂, PE:EA=1:1, R_{f}=0.50) showed the reaction was completed. The reaction mixture was quenched by addition water 10 mL at 25° C., and then extracted with DCM 30 mL (10 mL*3). The combined organic layers were washed with brine 30 mL, dried over Na₂SO₄, filtered and concentrated under reduced pressure to give a residue. The residue was purified by column chromatography (SiO₂, Petroleum ether/Ethyl acetate=3/1 to 2/1). 8 (420 mg, 1.13 mmol, 75.61% yield, 92.97% purity) was obtained as a white solid.

LCMS: RT=0.389 min, MS cal.: 346.1, [M−H]⁺=347.1

HPLC: RT=2.233 min, purity: 92.97%

¹H NMR (400 MHz, DMSO-d6) δ=7.79 (d, J=5.2 Hz, 1H), 5.59 (s, 2H), 4.28 (s, 3H), 3.88 (d, J=10.0 Hz, 6H), 3.34 (s, 3H)

### General Procedure for Preparation of Scheme 1, Intermediate 9:

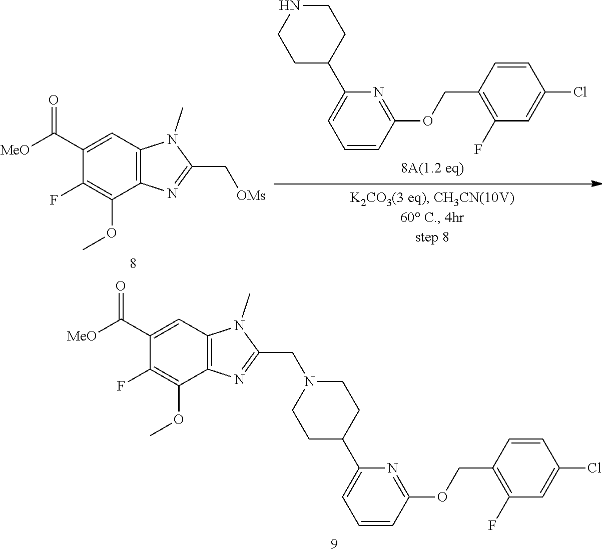 [SMILES: COC(=O)c1cc2c(nc(COS(C)(=O)=O)n2C)c(OC)c1F ; Fc1cc(Cl)ccc1COc1cccc(C2CCNCC2)n1 ; COC(=O)c1cc2c(nc(CN3CCC(c4cccc(OCc5ccc(Cl)cc5F)n4)CC3)n2C)c(OC)c1F]

To a mixture of Compound 8 (100 mg, 288.74 μmol, 1 eq), 8A (111.15 mg, 346.49 μmol, 1.2 eq) and K₂CO₃ (119.72 mg, 866.22 μmol, 3 eq) in ACN (3 mL) was stirred at 60° C. for 1 hour. LCMS showed 8 was consumed completely and one main peak with desired mass was detected. The mixture was filtered and concentrated in vacuum. The residue was purified by prep-TLC (SiO₂, PE:EA=1:2, R_{f}=0.45). 9 (110 mg, 187.92 μmol, 65.08% yield) was obtained as a yellow oil.

LCMS: RT=0.490 min, MS cal.: 570.2, [M+H]⁺=571.1

HPLC: product: RT=3.388 min, purity: 97.54%

¹H NMR (400 MHz, DMSO-d6) δ=7.71 (d, J=5.2 Hz, 1H), 7.62 (t, J=7.6 Hz, 1H), 7.55 (t, J=8.0 Hz, 1H), 7.44 (dd, J=2.0, 10.0 Hz, 1H), 7.28 (dd, J=1.6, 8.4 Hz, 1H), 6.86 (d, J=7.2 Hz, 1H), 6.66 (d, J=8.4 Hz, 1H), 5.36 (s, 2H), 4.26 (s, 3H), 3.91 (s, 3H), 3.87 (s, 3H), 3.83 (s, 2H), 2.93 (br d, J=11.2 Hz, 2H), 2.64-2.55 (m, 1H), 2.22 (br t, J=9.6 Hz, 2H), 1.82-1.63 (m, 4H)

### General Procedure for Preparation of Scheme 1, Compound 337 (10):

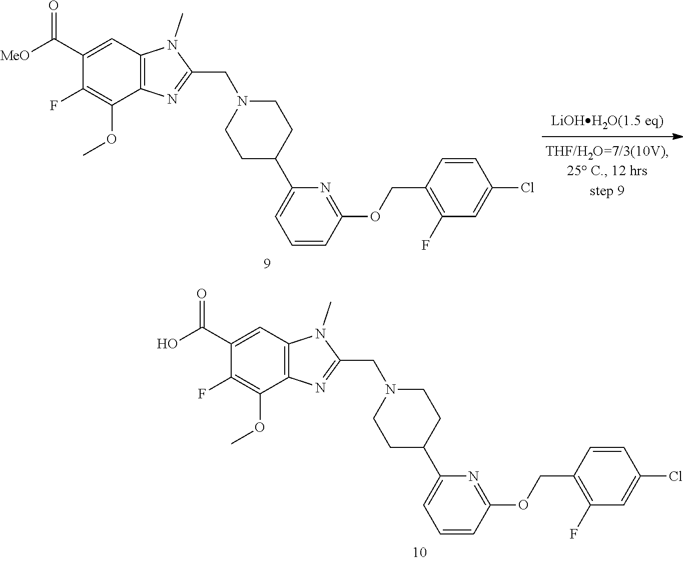 [SMILES: COC(=O)c1cc2c(nc(CN3CCC(c4cccc(OCc5ccc(Cl)cc5F)n4)CC3)n2C)c(OC)c1F ; COc1c(F)c(C(=O)O)cc2c1nc(CN1CCC(c3cccc(OCc4ccc(Cl)cc4F)n3)CC1)n2C]

To a mixture of 9 (110 mg, 192.64 μmol, 1 eq) in THF (2.1 mL) and H₂O (0.9 mL) was added LiOH·H₂O (12.13 mg, 288.96 μmol, 1.5 eq). The mixture was stirred at 25° C. for 12 hours. LC-MS showed 9 was consumed completely and one main peak with desired mass was detected. The mixture was filtered and concentrated in vacuum. The residue was purified by prep-HPLC (column: Waters Xbridge Prep OBD C₁₈ 150*40 mm*10 um; mobile phase: [H₂O (10 mM NH₄HCO₃)-ACN]; gradient: 20%-70% B over 8.0 min). 2-((4-(6-((4-Chloro-2-fluorobenzyl)oxy)pyridin-2-yl)piperidin-1-yl)methyl)-5-fluoro-4-methoxy-1-methyl-1H-benzo[d]imidazole-6-carboxylic acid (28.48 mg, 51.00 μmol, 26.47% yield) was obtained as a white solid.

LCMS: RT=2.696 min, MS cal.: 556.2, [M+H]⁺=557.2

HPLC: product: RT=11.591 min, purity: 99.74%

¹H NMR (400 MHz, DMSO-d6) δ=7.68 (d, J=5.2 Hz, 1H), 7.62 (t, J=8.0 Hz, 1H), 7.55 (t, J=8.4 Hz, 1H), 7.45 (dd, J=1.6, 10.0 Hz, 1H), 7.28 (dd, J=1.6, 8.4 Hz, 1H), 6.86 (d, J=7.2 Hz, 1H), 6.66 (d, J=8.4 Hz, 1H), 5.35 (s, 2H), 4.25 (s, 3H), 3.89 (s, 3H), 3.82 (s, 2H), 2.93 (br d, J=11.2 Hz, 2H), 2.63-2.53 (m, 1H), 2.21 (br t, J=10.4 Hz, 2H), 1.83-1.62 (m, 4H)

### General Procedure for Preparation of Scheme 2, Intermediate 7A:

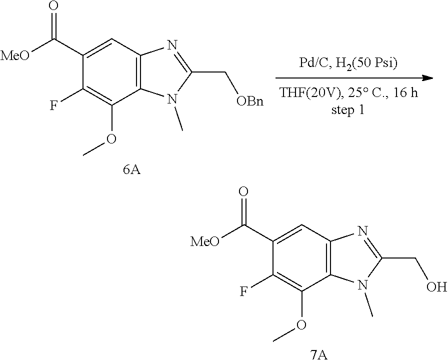 [SMILES: COC(=O)c1cc2nc(COCc3ccccc3)n(C)c2c(OC)c1F ; COC(=O)c1cc2nc(CO)n(C)c2c(OC)c1F]

To a solution of 6A (500 mg, 1.40 mmol, 1 eq) in MeOH (10 mL) was added Pd/C (148.48 mg, 139.52 μmol, 10% purity, 0.1 eq) under N₂ atmosphere. The suspension was degassed and purged with H₂ for 3 times. The mixture was stirred under H₂ (50 Psi) at 25° C. for 48 hr. LCMS showed 6A was consumed completely and one main peak with desired mass was detected. The mixture was filtered and concentrated in vacuum. The crude product was used into the next step without further purification. 7A (305 mg, 1.02 mmol, 73.35% yield) was obtained as a yellow solid.

LCMS: RT=0.296 min, MS cal.: 268.1 [M+H]⁺=269.0

HPLC: product: RT=1.544 min, purity: 90.69%

¹H NMR (400 MHz, DMSO-d6) δ=7.83 (d, J=5.6 Hz, 1H), 5.79 (br s, 1H), 4.69 (s, 2H), 4.00 (s, 6H), 3.85 (s, 3H)

### General Procedure for Preparation of Scheme 2, Intermediate 8A:

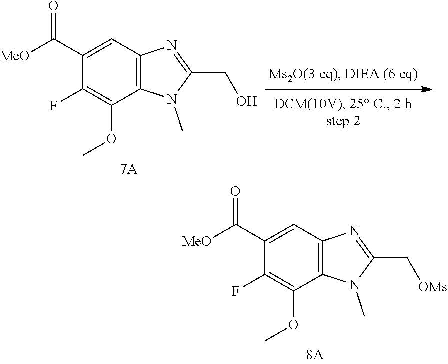 [SMILES: COC(=O)c1cc2nc(CO)n(C)c2c(OC)c1F ; COC(=O)c1cc2nc(COS(C)(=O)=O)n(C)c2c(OC)c1F]

To a solution of 7A (300 mg, 1.12 mmol, 1 eq) and DIEA (867.27 mg, 6.71 mmol, 1.17 mL, 6 eq) in DCM (10 mL) was added methylsulfonyl methanesulfonate (584.46 mg, 3.36 mmol, 3 eq) at 0° C. The resulting mixture was stirred at 25° C. for 1 hr. LCMS showed 7A was consumed completely and one main peak with desired mass was detected. The residue was poured into water (100 mL). The aqueous phase was extracted with dichloromethane (10 mL*3). The combined organic phase was washed with saturated brine (50 mL*1), dried with anhydrous Na₂SO₄, filtered and concentrated in vacuum. The residue was purified by column chromatography (SiO₂, Petroleum ether:Ethyl acetate=1/0 to 0/1). 8A (160 mg, 461.99 μmol, 41.31% yield) was obtained as a yellow solid.

LCMS: RT=0.390 min, MS cal.: 346.1 [M+H]⁺=347.1

¹H NMR (400 MHz, CHCl3-d) δ=8.07 (d, J=5.6 Hz, 1H), 5.47 (s, 2H), 4.13-4.10 (m, 6H), 3.96 (s, 3H), 3.11 (s, 3H)

### General Procedure for Preparation of Scheme 2, Intermediate 9A:

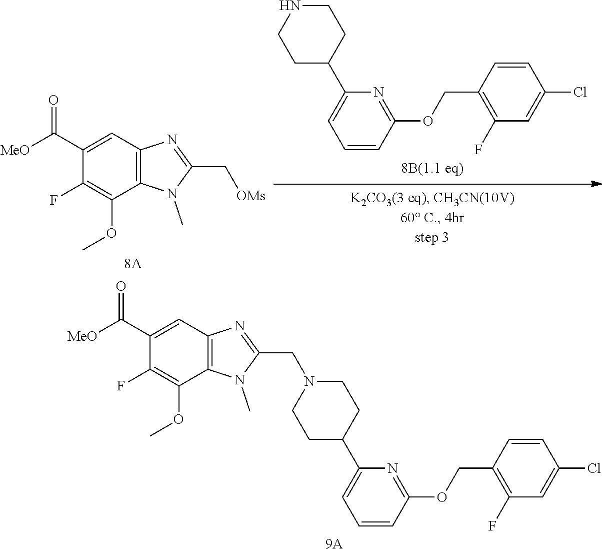 [SMILES: COC(=O)c1cc2nc(COS(C)(=O)=O)n(C)c2c(OC)c1F ; Fc1cc(Cl)ccc1COc1cccc(C2CCNCC2)n1 ; COC(=O)c1cc2nc(CN3CCC(c4cccc(OCc5ccc(Cl)cc5F)n4)CC3)n(C)c2c(OC)c1F]

To a mixture of 8A (70 mg, 202.12 μmol, 1 eq), 8B (77.80 mg, 242.54 μmol, 1.2 eq) and K₂CO₃ (83.80 mg, 606.36 μmol, 3 eq) in ACN (2 mL) was stirred at 60° C. for 1 hour. TLC (SiO₂, PE:EA=1:2, Rf=0.40) showed the reaction was completed. The mixture was filtered and concentrated in vacuum. The residue was purified by prep-TLC (SiO₂, Petroleum ether:Ethyl acetate=1:2, R_{f}=0.40). 9A (110 mg, 190.52 μmol, 94.26% yield) was obtained as a yellow oil.

LCMS: RT=0.474 min, MS cal.: 570.2 [M+H]⁺=571.4

HPLC: product: RT=3.378 min, purity: 98.90%

¹H NMR (400 MHz, DMSO-d6) δ=7.83 (d, J=5.6 Hz, 1H), 7.62 (t, J=7.6 Hz, 1H), 7.56 (t, J=8.0 Hz, 1H), 7.44 (dd, J=1.6, 9.6 Hz, 1H), 7.28 (dd, J=1.6, 8.0 Hz, 1H), 6.86 (d, J=7.2 Hz, 1H), 6.67 (d, J=8.0 Hz, 1H), 5.35 (s, 2H), 4.06 (s, 3H), 4.02 (s, 3H), 3.86 (s, 3H), 3.78 (s, 2H), 2.93 (br d, J=11.2 Hz, 2H), 2.64-2.54 (m, 1H), 2.19 (br t, J=9.6 Hz, 2H), 1.79 (br s, 1H), 1.75-1.64 (m, 3H)

### General Procedure for Preparation of Compound 338 (10A):

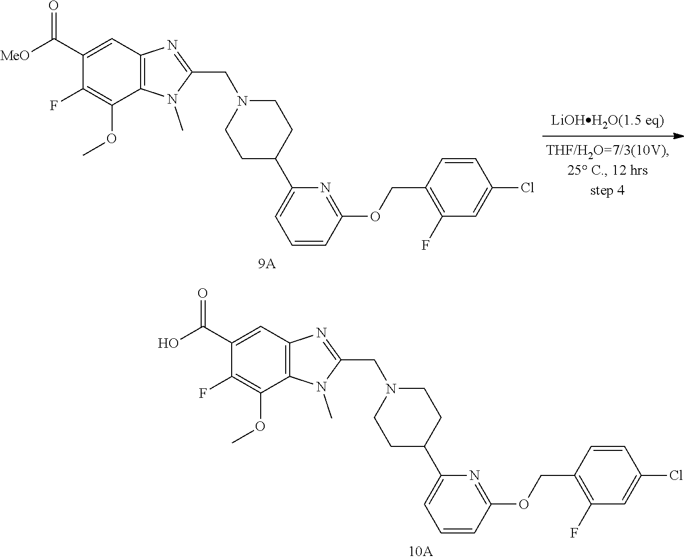 [SMILES: COC(=O)c1cc2nc(CN3CCC(c4cccc(OCc5ccc(Cl)cc5F)n4)CC3)n(C)c2c(OC)c1F ; COc1c(F)c(C(=O)O)cc2nc(CN3CCC(c4cccc(OCc5ccc(Cl)cc5F)n4)CC3)n(C)c12]

To a mixture of 9A (100 mg, 175.13 μmol, 1 eq) in THF (3.5 mL) and H₂O (0.9 mL) was added LiOH·H₂O (11.02 mg, 262.69 μmol, 1.5 eq). The mixture was stirred at 25° C. for 12 hours. LC-MS showed 9A was consumed completely and one main peak with desired mass was detected. The mixture was concentrated in vacuum. The residue was purified by prep-HPLC (column: Waters Xbridge Prep OBD C₁₈ 150*40 mm*10 um; mobile phase: [H₂O (10 mM NH₄HCO₃)-ACN]; gradient: 20%-70% B over 8.0 min). 2-((4-(6-((4-Chloro-2-fluorobenzyl)oxy)pyridin-2-yl)piperidin-1-yl)methyl)-6-fluoro-7-methoxy-1-methyl-1H-benzo[d]imidazole-5-carboxylic acid (28.23 mg, 50.09 μmol, 28.60% yield) was obtained as a yellow solid.

LCMS: RT=2.786 min, MS cal.: 556.2, [M+H]⁺=557.2

HPLC: product: RT=11.035 min, purity: 98.83%

¹H NMR (400 MHz, MeOH-d4) δ=7.76 (br d, J=3.2 Hz, 1H), 7.57 (dd, J=7.6, 8.0 Hz, 1H), 7.49 (t, J=8.4 Hz, 1H), 7.22-7.15 (m, 2H), 6.82 (d, J=7.2 Hz, 1H), 6.63 (d, J=8.0 Hz, 1H), 5.40 (s, 2H), 4.14 (s, 3H), 4.08 (d, J=1.2 Hz, 3H), 3.85 (s, 2H), 3.05 (br d, J=10.8 Hz, 2H), 2.65 (quin, J=7.6 Hz, 1H), 2.40-2.26 (m, 2H), 1.96-1.75 (m, 4H)

### Example A73—Preparation of Compound 339. 2-((4-(3-((4-Cyano-2-fluorobenzyl)oxy)-4-fluorophenyl)piperidin-1-yl)methyl)-7-fluoro-4-methoxy-1-methyl-1H-benzo[d]imidazole-6-carboxylic acid (Compound 339) (5)

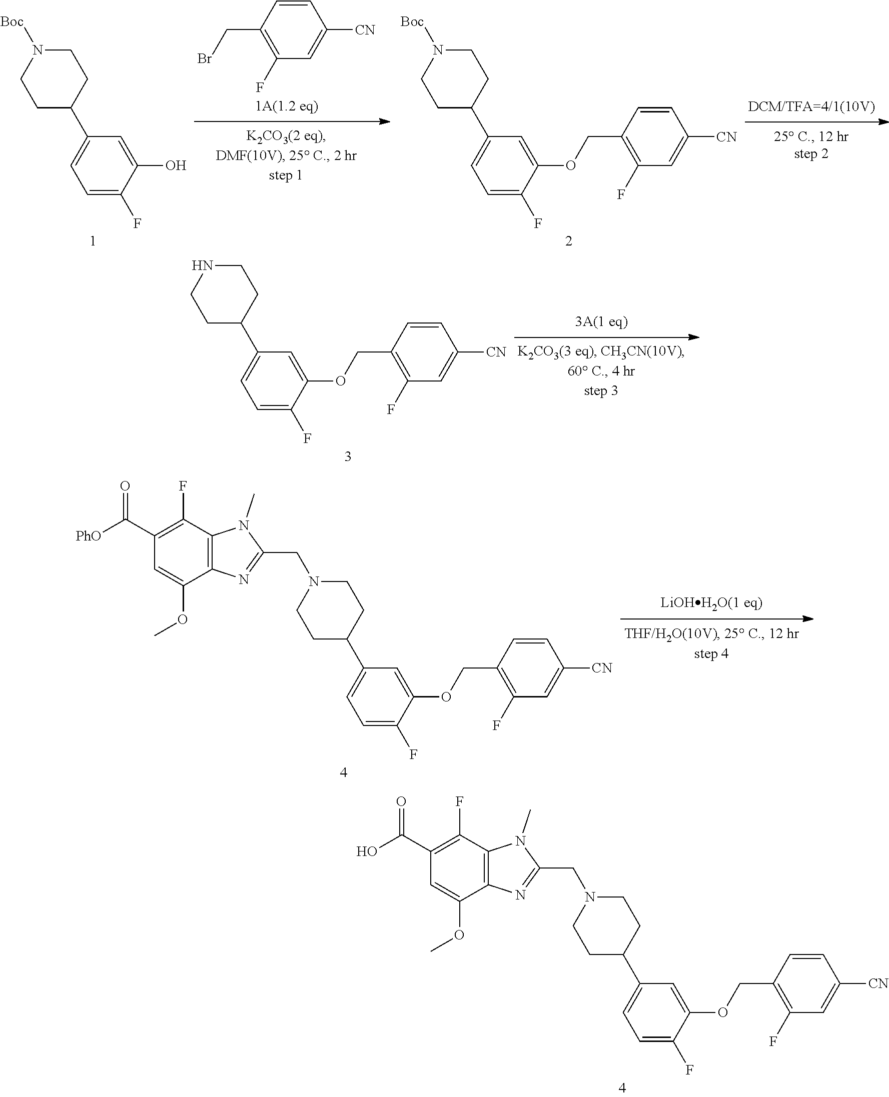 [SMILES: CC(C)(C)OC(=O)N1CCC(c2ccc(F)c(O)c2)CC1 ; N#Cc1ccc(CBr)c(F)c1 ; CC(C)(C)OC(=O)N1CCC(c2ccc(F)c(OCc3ccc(C#N)cc3F)c2)CC1 ; N#Cc1ccc(COc2cc(C3CCNCC3)ccc2F)c(F)c1 ; COc1cc(C(=O)Oc2ccccc2)c(F)c2c1nc(CN1CCC(c3ccc(F)c(OCc4ccc(C#N)cc4F)c3)CC1)n2C ; COc1cc(C(=O)O)c(F)c2c1nc(CN1CCC(c3ccc(F)c(OCc4ccc(C#N)cc4F)c3)CC1)n2C]

### General Procedure for Preparation of Intermediate 2:

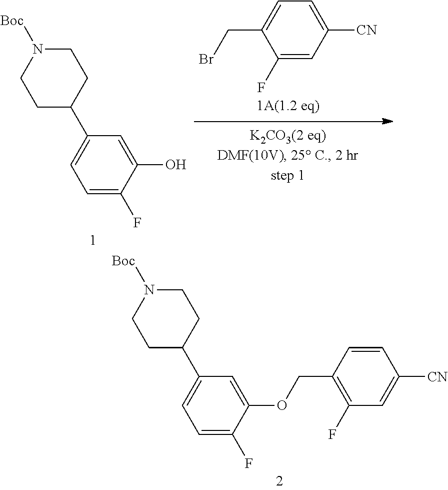

To a mixture of 1 (200 mg, 677.17 μmol, 1 eq) and 1A (173.92 mg, 812.60 μmol, 1.2 eq) in DMF (3 mL) was added K₂CO₃ (187.18 mg, 1.35 mmol, 2 eq) in one portion at 20° C. under N₂. The mixture was de-gassed under reduced pressure and recharged with N₂. The mixture was stirred at 20° C. for 12 hours. The solution changed to cloudy. LCMS showed Compound 1 was consumed and a major peak was desired product. Filtrate, the mixture was concentrated in vacuum. The crude product was purified by prep-TLC (Petroleum ether:Ethyl acetate=5:1, Rf=0.6). 2 (160 mg, 373.42 μmol, 55.14% yield) as a white solid.

LCMS: RT=0.640 min, MS cal.: 428.2, [M+Na]⁺=451.3

¹H NMR (400 MHz, MeOH-d4) δ=7.96-7.91 (m, 1H), 7.79-7.74 (m, 2H), 7.20 (dd, J=1.6, 8.0 Hz, 1H), 7.14 (dd, J=8.4, 11.6 Hz, 1H), 6.84 (ddd, J=2.0, 4.4, 8.4 Hz, 1H), 5.31 (s, 2H), 4.07 (br d, J=11.2 Hz, 2H), 2.90-2.70 (m, 2H), 2.69-2.62 (m, 1H), 1.72 (br d, J=12.0 Hz, 2H), 1.56-1.46 (m, 2H), 1.41 (s, 9H)

### General Procedure for Preparation of Intermediate 3:

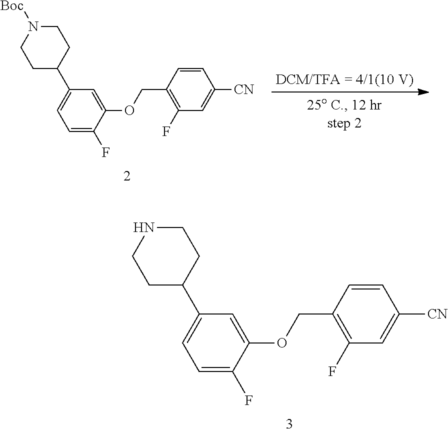 [SMILES: CC(C)(C)OC(=O)N1CCC(c2ccc(F)c(OCc3ccc(C#N)cc3F)c2)CC1 ; N#Cc1ccc(COc2cc(C3CCNCC3)ccc2F)c(F)c1]

To a mixture of 2 (160 mg, 373.42 μmol, 1 eq) in DCM (2 mL) was added TFA (767.50 mg, 6.73 mmol, 0.5 mL, 18.03 eq) in one portion at 20° C. under N₂. The mixture was stirred at 20° C. for 2 hours. LCMS showed the 2 was consumed and a main peak was desired product. To a mixture was added Saturated sodium carbonate solution to PH=9˜10. The aqueous phase was extracted with ethyl acetate (5 mL*3). The combined organic phase was washed with brine (10 mL*2), dried with anhydrous Na₂SO₄, filtered and concentrated in vacuum. The product was used to next step without purification. 3 (110 mg, 335.00 μmol, 89.71% yield) as a white solid.

LCMS: RT=0.377 min, MS cal.: 328.1, [M+H−(t-Bu)]⁺=329.1

¹H NMR (400 MHz, MeOH-d4) δ=7.80-7.73 (m, 1H), 7.62 (s, 1H), 7.60 (s, 1H), 7.07-7.01 (m, 2H), 6.85 (ddd, J=2.4, 4.4, 8.4 Hz, 1H), 5.28 (s, 2H), 3.12 (br d, J=12.4 Hz, 2H), 2.75-2.59 (m, 3H), 1.78 (br d, J=13.2 Hz, 2H), 1.60 (dq, J=4.0, 12.4 Hz, 2H)

### General Procedure for Preparation of Intermediate 4:

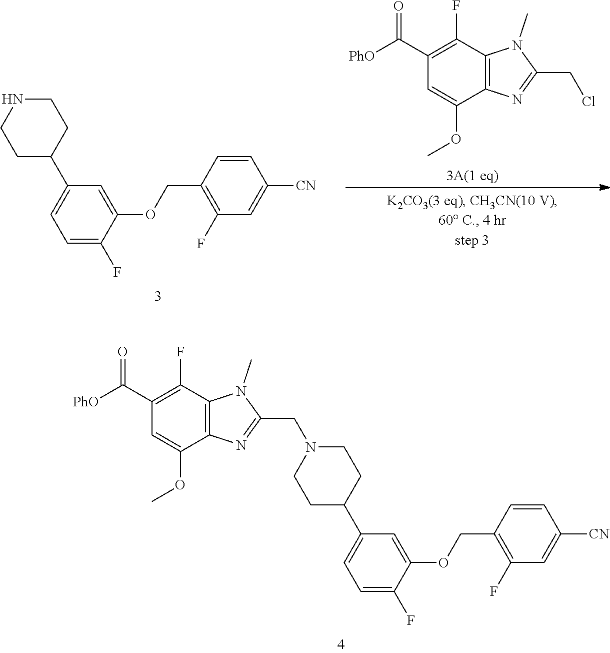 [SMILES: N#Cc1ccc(COc2cc(C3CCNCC3)ccc2F)c(F)c1 ; COc1cc(C(=O)Oc2ccccc2)c(F)c2c1nc(CCl)n2C ; COc1cc(C(=O)Oc2ccccc2)c(F)c2c1nc(CN1CCC(c3ccc(F)c(OCc4ccc(C#N)cc4F)c3)CC1)n2C]

To a mixture of 3 (51.78 mg, 157.70 μmol, 1.1 eq) and 3A (50 mg, 143.37 μmol, 1 eq) in ACN (2 mL) was added K₂CO₃ (59.44 mg, 430.10 μmol, 3 eq) in one portion at 20° C. under N₂. The mixture was stirred at 20° C. for 2 hours. The solution changed to cloudy. LCMS showed the 3A was consumed and a main peak was desired product. Filtrate, the mixture was concentrated in vacuum. The crude product was purified by prep-TLC (Petroleum ether:Ethyl acetate=1:1, R_{f}=0.25). 3 (65 mg, 101.46 μmol, 70.77% yield) as a white solid.

LCMS: RT=1.637 min, MS cal.: 640.2, [M+H]⁺=641.2

¹H NMR (400 MHz, MeOH-d4) δ=7.93 (d, J=10.4 Hz, 1H), 7.79-7.74 (m, 2H), 7.53-7.45 (m, 2H), 7.36-7.27 (m, 3H), 7.21-7.10 (m, 3H), 6.88-6.82 (m, 1H), 5.30 (s, 2H), 4.08 (s, 3H), 3.96 (s, 3H), 3.83 (s, 2H), 2.95 (br d, J=11.2 Hz, 2H), 2.55 (br d, J=3.6 Hz, 1H), 2.24-2.16 (m, 2H), 1.77-1.59 (m, 4H)

### General Procedure for Preparation of Compound 339 (5):

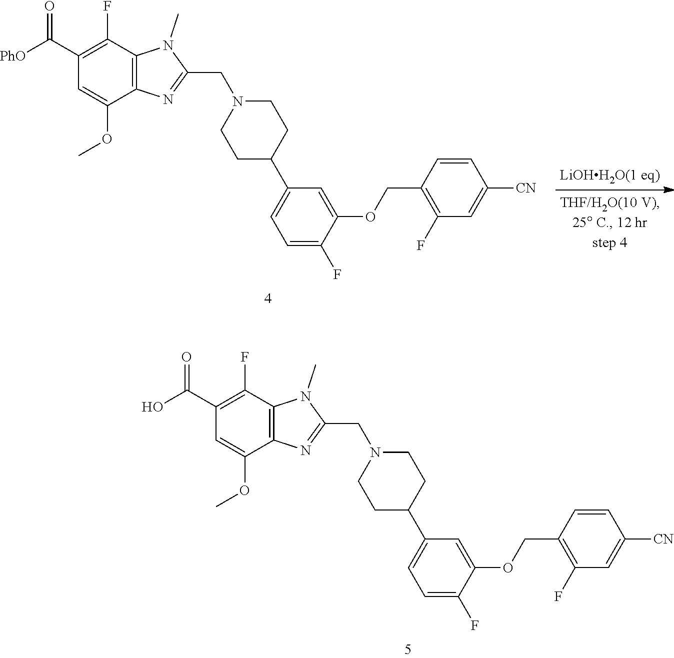 [SMILES: COc1cc(C(=O)Oc2ccccc2)c(F)c2c1nc(CN1CCC(c3ccc(F)c(OCc4ccc(C#N)cc4F)c3)CC1)n2C ; COc1cc(C(=O)O)c(F)c2c1nc(CN1CCC(c3ccc(F)c(OCc4ccc(C#N)cc4F)c3)CC1)n2C]

To a mixture of 4 (65 mg, 101.46 μmol, 1 eq) in THF (0.7 mL) and H₂O (0.3 mL) was added LiOH·H₂O (4.26 mg, 101.46 μmol, 1 eq) in one portion at 20° C. under N₂. The mixture was stirred at 20° C. for 12 hours. The color of the solution changed to yellow. LCMS showed a main peak was desired product. The mixture was concentrated in vacuum. The crude product was purified by prep-HPLC (column: Waters Xbridge Prep OBD C₁₈ 150*40 mm*10 um; mobile phase: [H₂O (10 mM NH₄HCO₃)-ACN]; gradient: 15%-65% B over 8.0 min). 2-((4-(3-((4-Cyano-2-fluorobenzyl)oxy)-4-fluorophenyl)piperidin-1-yl)methyl)-7-fluoro-4-methoxy-1-methyl-1H-benzo[d]imidazole-6-carboxylic acid (27.21 mg, 48.20 μmol, 47.50% yield) as a white solid.

LCMS: RT=2.546 min, MS cal.: 564.2, [M+H]⁺=565.2

HPLC: RT=8.102 min, purity: 99.48%

¹H NMR (400 MHz, MeOH-d4) δ=7.79-7.72 (m, 1H), 7.61 (d, J=8.8 Hz, 2H), 7.11 (d, J=4.8 Hz, 1H), 7.07-6.99 (m, 2H), 6.85 (ddd, J=2.0, 4.4, 8.4 Hz, 1H), 5.27 (s, 2H), 4.13 (s, 3H), 3.98 (s, 3H), 3.87 (s, 2H), 3.03 (br d, J=11.4 Hz, 2H), 2.60-2.50 (m, 1H), 2.36-2.28 (m, 2H), 1.85-1.71 (m, 4H)

### Example A74—Preparation of Compound 340. 2-((4-(3-((4-Chloro-2-fluorobenzyl)oxy)-4-fluorophenyl)piperidin-1-yl)methyl)-7-fluoro-4-methoxy-1-methyl-1H-benzo[d]imidazole-6-carboxylic acid (Compound 340) (7)

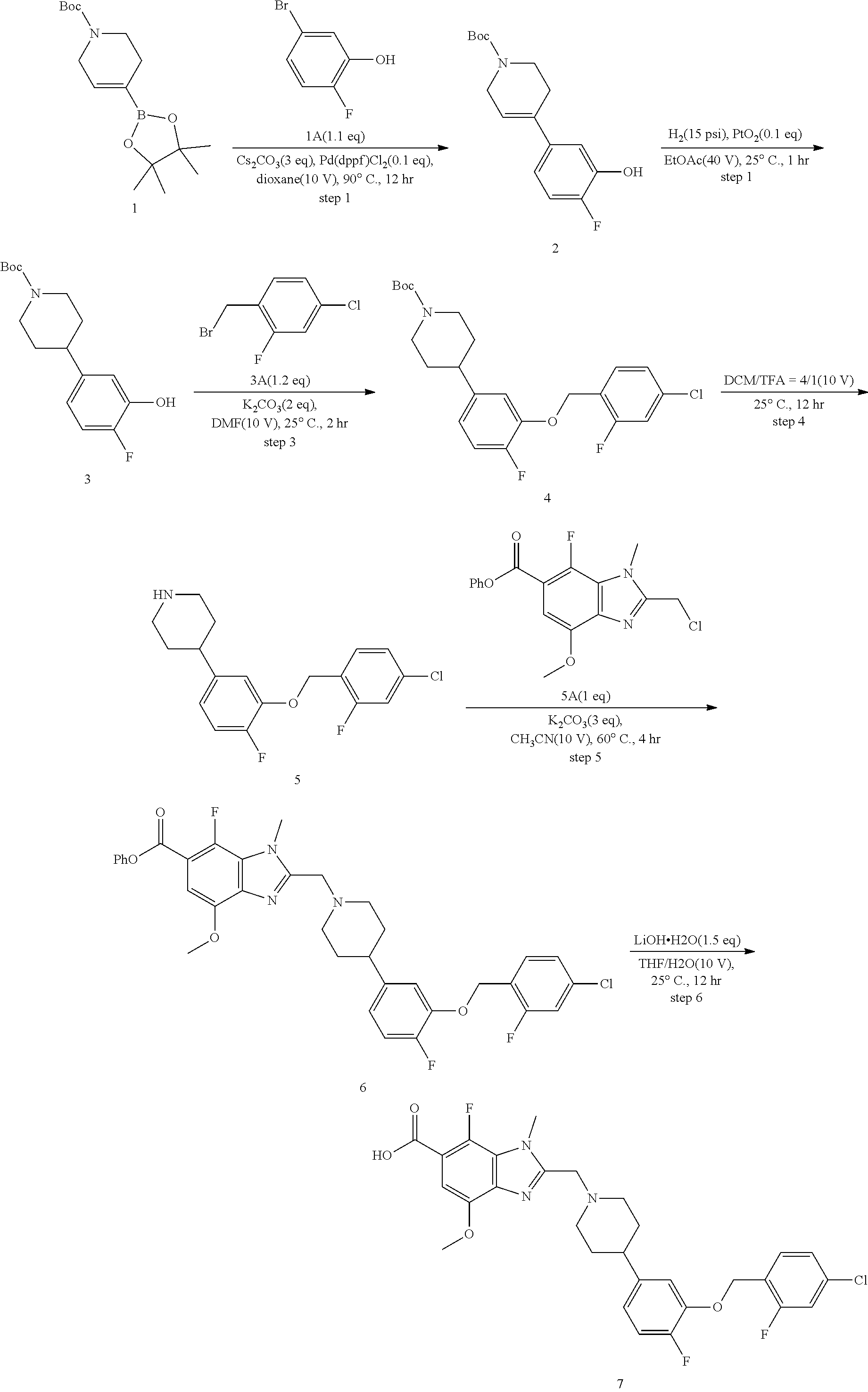

### General Procedure for Preparation of Intermediate 2:

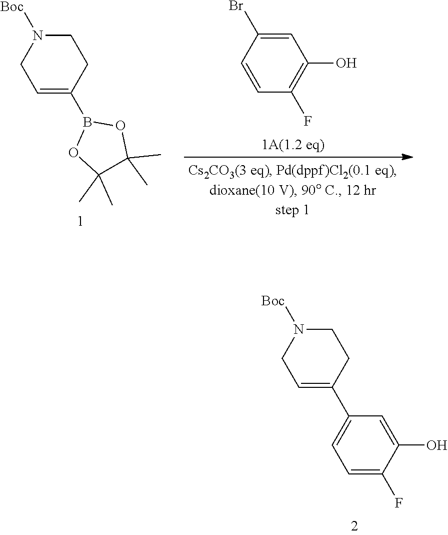 [SMILES: CC(C)(C)OC(=O)N1CC=C(B2OC(C)(C)C(C)(C)O2)CC1 ; Oc1cc(Br)ccc1F ; CC(C)(C)OC(=O)N1CC=C(c2ccc(F)c(O)c2)CC1]

To a mixture of 1 (1 g, 5.24 mmol, 1 eq) and 1A (1.94 g, 6.28 mmol, 1.2 eq) in dioxane (10 mL) was added Cs₂CO₃ (5.12 g, 15.71 mmol, 3 eq) and Pd(dppf)Cl₂ (383.10 mg, 523.57 μmol, 0.1 eq) in one portion at 20° C. under N₂. The mixture was de-gassed under reduced pressure and recharged with N₂. The mixture was stirred at 90° C. for 12 hours. The color of the solution changed to black. LC-MS showed 1 was consumed completely and one main peak with desired mass was detected. Filtrate, the mixture was concentrated in vacuum. The residue was purified by silica gel chromatography (column height: 250 mm, diameter: 100 mm, 100-200 mesh silica gel, Petroleum ether/Ethyl acetate=100/0, 20/80, 0/100). 2 (1 g, 3.41 mmol, 65.11% yield) as a white solid.

LCMS: RT=0.518 min, MS cal.: 293.1, [M+H−(Me)]⁺=279.2

¹H NMR (400 MHz, MeOH-d4) δ=9.82 (s, 1H), 7.08 (dd, J=8.4, 11.2 Hz, 1H), 6.98 (dd, J=2.4, 8.4 Hz, 1H), 6.84 (ddd, J=2.4, 4.4, 8.4 Hz, 1H), 6.02 (br s, 1H), 3.96 (br s, 2H), 3.51 (t, J=5.6 Hz, 2H), 2.38 (br d, J=1.6 Hz, 2H), 1.42 (s, 9H)

### General Procedure for Preparation of Intermediate 3:

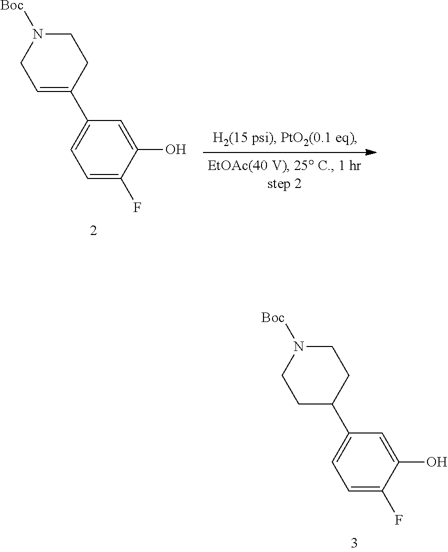 [SMILES: CC(C)(C)OC(=O)N1CC=C(c2ccc(F)c(O)c2)CC1 ; CC(C)(C)OC(=O)N1CCC(c2ccc(F)c(O)c2)CC1]

To a mixture of 2 (1 g, 3.41 mmol, 1 eq) in EtOAc (40 mL) was added PtO₂ (77.41 mg, 340.91 μmol, 0.1 eq) in one portion at 20° C. under N₂. The mixture was de-gassed under reduced pressure and recharged with H₂ (15 Psi). The mixture was stirred at 20° C. for 2 hours. The solution changed to cloudy. LCMS showed the 2 was consumed and a main peak was desired product. Filtrate, the mixture was concentrated in vacuum. The crude product was used no next step without purification. 3 (900 mg, 3.05 mmol, 89.39% yield) as a colorless oil.

LCMS: RT=0.528 min, MS cal.: 295.2, [M+H−(Me)]⁺=281.2

¹H NMR (400 MHz, MeOH-d4) δ=6.94 (dd, J=8.4, 11.2 Hz, 1H), 6.77 (dd, J=2.0, 8.4 Hz, 1H), 6.65 (ddd, J=2.0, 4.0, 8.0 Hz, 1H), 4.18 (br d, J=13.6 Hz, 2H), 2.84 (br s, 2H), 2.62 (tt, J=3.6, 12.0 Hz, 1H), 1.78 (br d, J=13.2 Hz, 2H), 1.57-1.49 (m, 2H), 1.47 (s, 9H)

### General Procedure for Preparation of Intermediate 4:

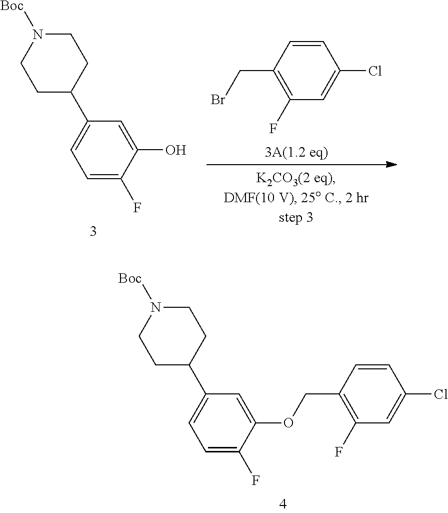 [SMILES: CC(C)(C)OC(=O)N1CCC(c2ccc(F)c(O)c2)CC1 ; Fc1cc(Cl)ccc1CBr ; CC(C)(C)OC(=O)N1CCC(c2ccc(F)c(OCc3ccc(Cl)cc3F)c2)CC1]

To a mixture of 3 (200 mg, 677.17 μmol, 1 eq) and 1 3A (181.59 mg, 812.60 μmol, 1.2 eq) in DMF (3 mL) was added K₂CO₃ (187.18 mg, 1.35 mmol, 2 eq) in one portion at 20° C. under N₂. The mixture was de-gassed under reduced pressure and recharged with N₂. The mixture was stirred at 20° C. for 12 hours. The solution changed to cloudy. LCMS showed the 3 was consumed and a major peak was desired product, TLC (Petroleum ether:Ethyl acetate=5:1, R_{f}=0.6). Filtrate, the mixture was concentrated in vacuum. The crude product was purified by prep-TLC (Petroleum ether:Ethyl acetate=5:1, R_{f}=0.5). 4 (200 mg, 456.72 μmol, 67.45% yield) as a white solid.

LCMS: RT=0.707 min, MS cal.: 437.2, [M+H−(Me)]⁺=423.2

¹H NMR (400 MHz, MeOH-d4) δ=7.59 (t, J=8.0 Hz, 1H), 7.51 (dd, J=2.4, 10.0 Hz, 1H), 7.35 (dd, J=1.6, 8.0 Hz, 1H), 7.19 (dd, J=1.6, 8.0 Hz, 1H), 7.12 (dd, J=8.4, 11.6 Hz, 1H), 6.83 (ddd, J=2.0, 4.4, 8.4 Hz, 1H), 5.20 (s, 2H), 4.17-4.00 (m, 2H), 2.92-2.59 (m, 3H), 1.72 (br d, J=11.6 Hz, 2H), 1.56-1.46 (m, 2H), 1.41 (s, 9H)

### General Procedure for Preparation of Intermediate 5:

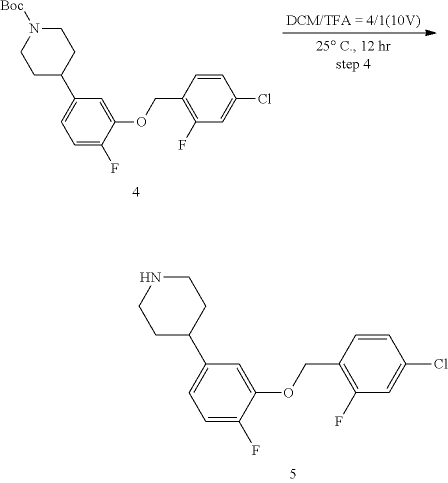 [SMILES: CC(C)(C)OC(=O)N1CCC(c2ccc(F)c(OCc3ccc(Cl)cc3F)c2)CC1 ; Fc1cc(Cl)ccc1COc1cc(C2CCNCC2)ccc1F]

To a mixture of 4 (200 mg, 456.72 μmol, 1 eq) a in DCM (2 mL) was added TFA (767.50 mg, 6.73 mmol, 0.5 mL, 14.74 eq) in one portion at 20° C. under N₂. The mixture was stirred at 20° C. for 1 hours. The color of the solution changed to yellow. LCMS showed the 4 was consumed and a main peak was desired product. To a mixture was added saturated sodium carbonate solution to PH=9˜10. The aqueous phase was extracted with ethyl acetate (5 mL*3). The combined organic phase was washed with brine (10 mL*2), dried with anhydrous Na₂SO₄, filtered and concentrated in vacuum. The crude product was used to next step without purification. 5 (140 mg, 414.46 μmol, 90.75% yield) as a white solid.

LCMS: RT=0.409 min, MS cal.: 337.1, [M+H]⁺=338.2

¹H NMR (400 MHz, CHCl3-d) δ=7.52 (t, J=8.0 Hz, 1H), 7.25 (br d, J=2.4 Hz, 1H), 7.23 (s, 1H), 7.05-6.98 (m, 2H), 6.83 (ddd, J=2.4, 4.4, 8.0 Hz, 1H), 5.17 (s, 2H), 3.12 (br d, J=12.4 Hz, 2H), 2.74-2.58 (m, 3H), 1.78 (br d, J=12.8 Hz, 2H), 1.65-1.54 (m, 2H).

### General Procedure for Preparation of Intermediate 6:

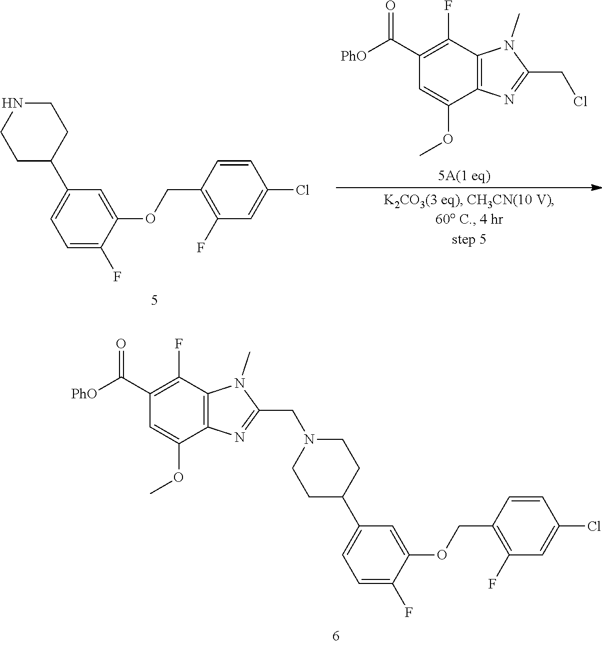 [SMILES: Fc1cc(Cl)ccc1COc1cc(C2CCNCC2)ccc1F ; COc1cc(C(=O)Oc2ccccc2)c(F)c2c1nc(CCl)n2C ; COc1cc(C(=O)Oc2ccccc2)c(F)c2c1nc(CN1CCC(c3ccc(F)c(OCc4ccc(Cl)cc4F)c3)CC1)n2C]

To a mixture of 5 (53.27 mg, 157.70 μmol, 1.1 eq) and 5A (50 mg, 143.37 μmol, 1 eq) in ACN (2 mL) was added K₂CO₃ (59.44 mg, 430.10 μmol, 3 eq) in one portion at 20° C. under N₂. The mixture was stirred at 20° C. for 2 hours. The solution changed to cloudy. LCMS showed the 5 was consumed and a main peak was desired product. Filtrate, the mixture was concentrated in vacuum. The crude product was prep-TLC (Petroleum ether:Ethyl acetate=1:1, R_{f}=0.25). 6 (70 mg, 107.68 μmol, 75.11% yield) as a white solid.

LCMS: RT=0.533 min, MS cal.: 649.2, [M+H]⁺=650.3

¹H NMR (400 MHz, CHCl3-d) δ=7.59 (t, J=8.0 Hz, 1H), 7.49 (t, J=7.6 Hz, 3H), 7.37-7.27 (m, 4H), 7.21-7.15 (m, 2H), 7.11 (dd, J=8.4, 11.2 Hz, 1H), 6.83 (br dd, J=3.6, 7.6 Hz, 1H), 5.19 (s, 2H), 4.08 (s, 3H), 3.96 (s, 3H), 3.84 (s, 2H), 2.95 (br d, J=10.8 Hz, 2H), 2.55 (br s, 1H), 2.21 (br t, J=11.6 Hz, 2H), 1.79-1.71 (m, 2H), 1.71-1.58 (m, 2H)

### General Procedure for Preparation of Compound 340 (7):

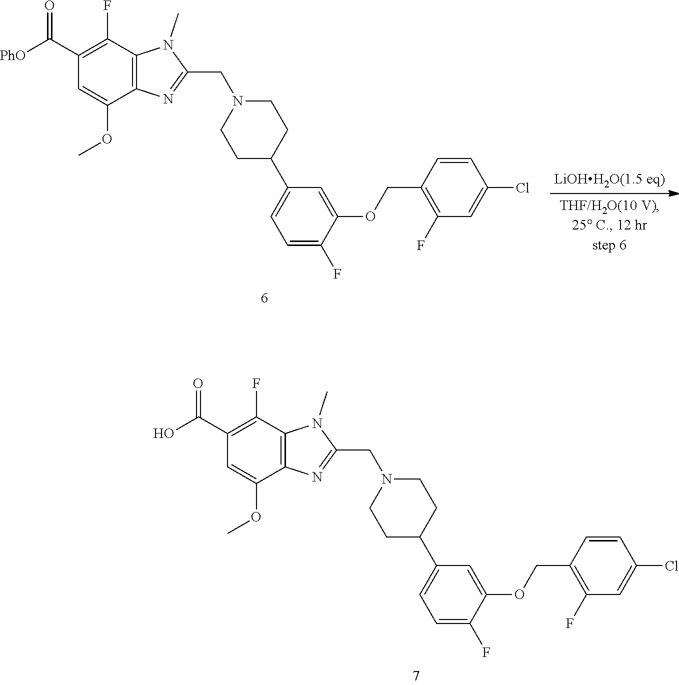 [SMILES: COc1cc(C(=O)Oc2ccccc2)c(F)c2c1nc(CN1CCC(c3ccc(F)c(OCc4ccc(Cl)cc4F)c3)CC1)n2C ; COc1cc(C(=O)O)c(F)c2c1nc(CN1CCC(c3ccc(F)c(OCc4ccc(Cl)cc4F)c3)CC1)n2C]

To a mixture of 6 (70 mg, 107.68 μmol, 1 eq) in THF (0.7 mL) and H₂O (0.3 mL) was added LiOH·H₂O (6.78 mg, 161.52 μmol, 1.5 eq) in one portion at 20° C. under N₂. The mixture was stirred at 20° C. for 12 hours. The color of the solution changed to yellow. LCMS showed the 6 was consumed and a main peak was desired product. The mixture was concentrated in vacuum. The crude product was purified by prep-HPLC (column: Waters Xbridge Prep OBD C₁₈ 150*40 mm*10 um; mobile phase: [H₂O (10 mM NH₄HCO₃)-ACN]; gradient: 20%-70% B over 8.0 min). 2-((4-(3-((4-Chloro-2-fluorobenzyl)oxy)-4-fluorophenyl)piperidin-1-yl)methyl)-7-fluoro-4-methoxy-1-methyl-1H-benzo[d]imidazole-6-carboxylic acid (29.33 mg, 51.10 μmol, 47.45% yield) as a white solid.

LCMS: RT=2.730 min, MS cal.: 573.2, [M+H]⁺=574.2

HPLC: RT=9.361 min, purity: 98.59%

¹H NMR (400 MHz, MeOH-d4) δ=7.52 (t, J=8.0 Hz, 1H), 7.24 (br dd, J=3.2, 8.0 Hz, 2H), 7.10 (d, J=4.8 Hz, 1H), 7.04-6.97 (m, 2H), 6.83 (ddd, J=1.6, 4.0, 8.4 Hz, 1H), 5.16 (s, 2H), 4.13 (s, 3H), 3.98 (s, 3H), 3.87 (s, 2H), 3.03 (br d, J=11.2 Hz, 2H), 2.60-2.48 (m, 1H), 2.39-2.25 (m, 2H), 1.84-1.68 (m, 4H)

### Example A75—Preparation of Compound 302. 2-((4-(6-((4-Chloro-2-fluorobenzyl)oxy)pyridin-2-yl)piperidin-1-yl)methyl)-7-fluoro-4-methoxy-1-methyl-1H-benzo[d]imidazole-6-carboxylic acid (Compound 302) (3)

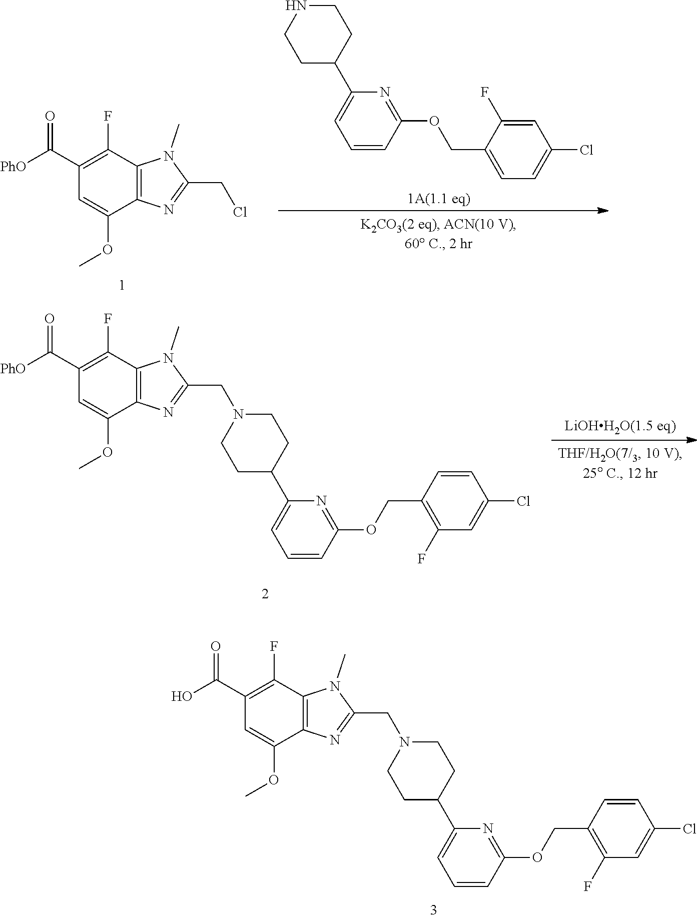 [SMILES: COc1cc(C(=O)Oc2ccccc2)c(F)c2c1nc(CCl)n2C ; Fc1cc(Cl)ccc1COc1cccc(C2CCNCC2)n1 ; COc1cc(C(=O)Oc2ccccc2)c(F)c2c1nc(CN1CCC(c3cccc(OCc4ccc(Cl)cc4F)n3)CC1)n2C ; COc1cc(C(=O)O)c(F)c2c1nc(CN1CCC(c3cccc(OCc4ccc(Cl)cc4F)n3)CC1)n2C]

### General Procedure for Preparation of Intermediate 2:

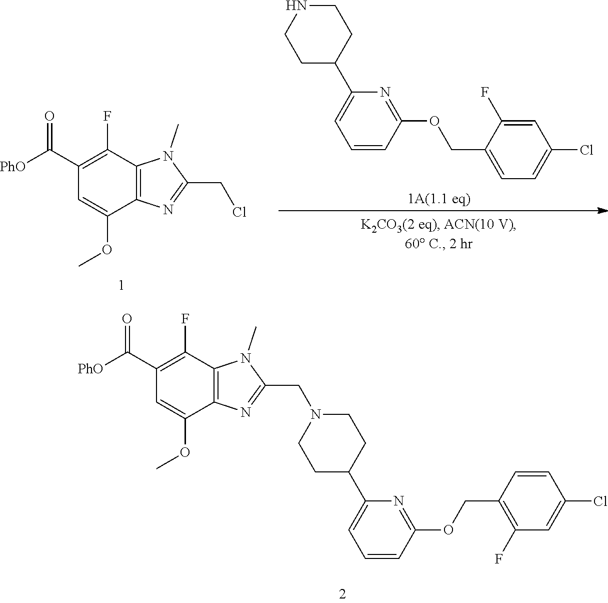 [SMILES: COc1cc(C(=O)Oc2ccccc2)c(F)c2c1nc(CCl)n2C ; Fc1cc(Cl)ccc1COc1cccc(C2CCNCC2)n1 ; COc1cc(C(=O)Oc2ccccc2)c(F)c2c1nc(CN1CCC(c3cccc(OCc4ccc(Cl)cc4F)n3)CC1)n2C]

A mixture of 1 (75 mg, 215.05 μmol, 1 eq), 1A (75.88 mg, 236.56 μmol, 1.1 eq), K₂CO₃ (59.44 mg, 430.10 μmol, 2 eq), in ACN (2 mL) was degassed and purged with N₂ for 3 times, and then the mixture was stirred at 60° C. for 2 hr under N₂ atmosphere. LC-MS (product. RT=1.866 min) showed 1 was consumed completely and one main peak with desired mass was detected. The reaction mixture was diluted with H₂O 15 mL and extracted with EtOAc 60 mL (20 mL*3). The combined organic layers were washed with brine 15 mL, dried over Na₂SO₄, filtered, and concentrated under reduced pressure to give a residue. The residue was purified by column chromatography (SiO₂, Petroleum ether/Ethyl acetate=10/1 to 1/1). 2 (100 mg, 157.96 μmol, 73.45% yield) was obtained as a yellow oil.

LCMS: RT=1.866 min, MS cal.: 632.20, [M+H]⁺=633.3

¹H NMR (400 MHz, CHCl3-d) δ=7.55-7.49 (m, 1H), 7.49-7.42 (m, 3H), 7.33-7.28 (m, 2H), 7.26-7.24 (m, 1H), 7.16-7.09 (m, 2H), 6.75 (d, J=7.2 Hz, 1H), 6.62 (d, J=8.4 Hz, 1H), 5.41 (s, 2H), 4.22 (br s, 3H), 4.06 (s, 3H), 4.02-3.82 (m, 1H), 3.37-2.87 (m, 2H), 2.77-2.58 (m, 1H), 2.51-2.18 (m, 2H), 2.03-1.79 (m, 3H), 1.74-1.38 (m, 2H)

### General Procedure for Preparation of Compound 302 (3):

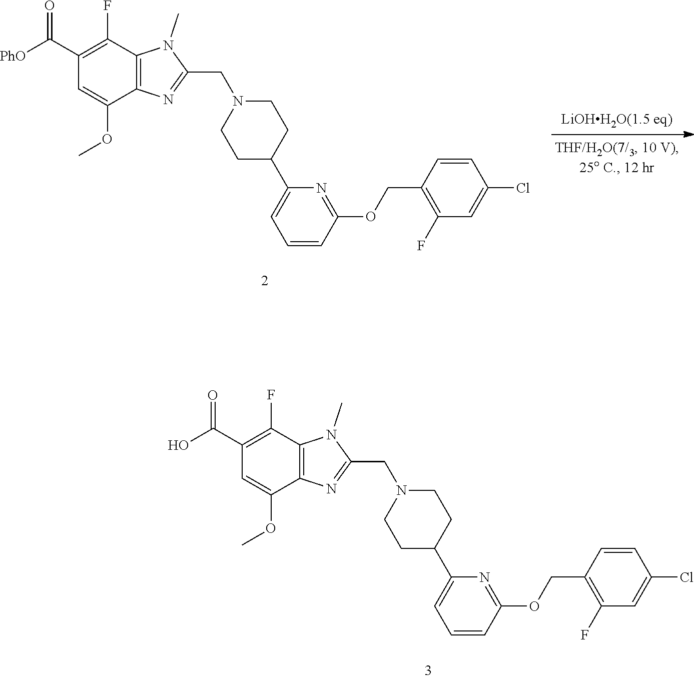 [SMILES: COc1cc(C(=O)Oc2ccccc2)c(F)c2c1nc(CN1CCC(c3cccc(OCc4ccc(Cl)cc4F)n3)CC1)n2C ; COc1cc(C(=O)O)c(F)c2c1nc(CN1CCC(c3cccc(OCc4ccc(Cl)cc4F)n3)CC1)n2C]

A mixture of 2 (90 mg, 142.16 μmol, 1 eq), LiOH·H₂O (8.95 mg, 213.24 μmol, 1.5 eq) in THF (0.7 mL) and H₂O (0.3 mL) was degassed and purged with N₂ for 3 times, and then the mixture was stirred at 25° C. for 12 hr under N₂ atmosphere. LC-MS (product, RT=1.177 min) showed 2 was consumed completely and one main peak with desired mass was detected. The reaction was purified directly. The residue was purified by prep-HPLC (column: Waters Xbridge Prep OBD C18 150*40 mm*10 um; mobile phase: [H₂O (10 mM NH₄HCO₃)-ACN]; gradient: 20%-70% B over 8.0 min). 2-((4-(6-((4-Chloro-2-fluorobenzyl)oxy)pyridin-2-yl)piperidin-1-yl)methyl)-7-fluoro-4-methoxy-1-methyl-1H-benzo[d]imidazole-6-carboxylic acid (26.57 mg, 47.46 μmol, 33.38% yield, 99.49% purity) was obtained as a white solid.

LCMS: RT=2.334 min, MS cal.: 556.17, [M+1]⁺=557.2

HPLC: RT=11.45 min, purity: 99.59%

¹H NMR (400 MHz, MeOH-d4) δ=7.61-7.53 (m, 1H), 7.49 (t, J=8.0 Hz, 1H), 7.22-7.15 (m, 2H), 7.07 (d, J=4.8 Hz, 1H), 6.81 (d, J=7.2 Hz, 1H), 6.62 (d, J=8.0 Hz, 1H), 5.40 (s, 2H), 4.13 (s, 3H), 3.97 (s, 3H), 3.86 (s, 2H), 3.02 (br d, J=11.2 Hz, 2H), 2.69-2.59 (m, 1H), 2.37-2.26 (m, 2H), 1.92-1.80 (m, 4H)

### Example A76—Preparation of Compound 303. 2-((4-(6-((4-Cyano-2-fluorobenzyl)oxy)pyridin-2-yl)piperidin-1-yl)methyl)-7-fluoro-4-methoxy-1-methyl-1H-benzo[d]imidazole-6-carboxylic acid (Compound 303) (9)

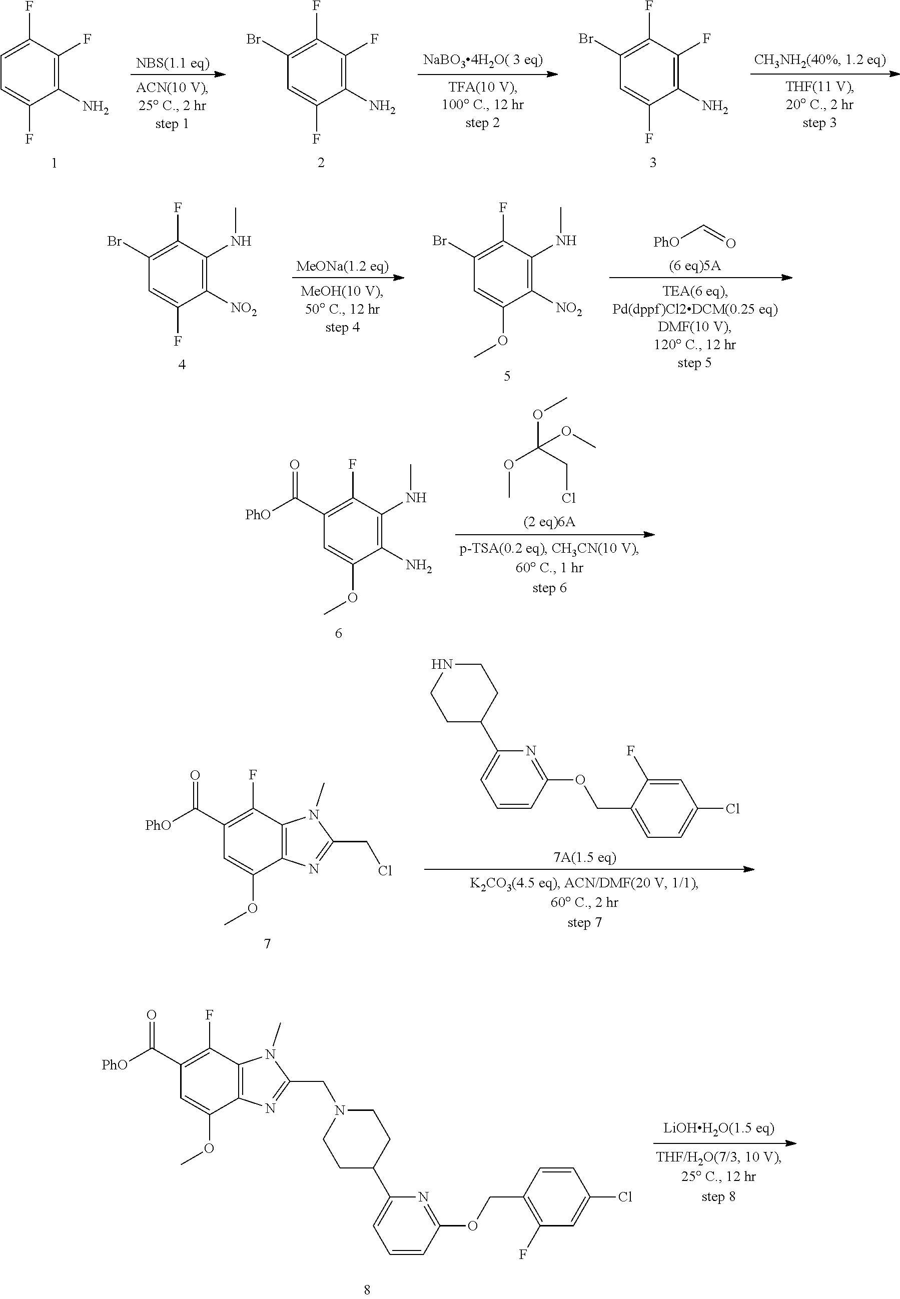

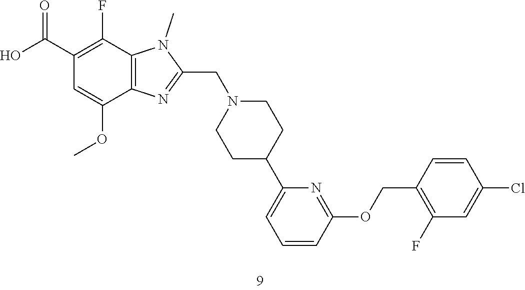 [SMILES: COc1cc(C(=O)O)c(F)c2c1nc(CN1CCC(c3cccc(OCc4ccc(Cl)cc4F)n3)CC1)n2C]

### General Procedure for Preparation of Intermediate 2:

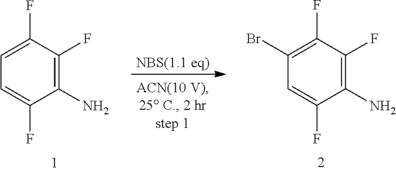 [SMILES: Nc1c(F)ccc(F)c1F ; Nc1c(F)cc(Br)c(F)c1F]

To a solution of 1 (15 g, 101.97 mmol, 1 eq) in ACN (150 mL) was added NBS (19.96 g, 112.17 mmol, 1.1 eq) at 0° C. The mixture was stirred at 25° C. for 2 hr. LCMS (Rt=1.134) showed Reactant 1 was consumed completely and desired mass was detected. The reaction mixture was added H₂O (300 mL) and extracted with DCM (100 mL*3). The combined organic layers were washed with brine (100 mL*2), dried over Na₂SO₄, filtered, and concentrated under reduced pressure to give a crude product. The residue was purified by column chromatography (SiO₂, Petroleum ether/Ethyl acetate-100/1 to 5/1). 2 (17.5 g, 77.44 mmol, 76% yield) was obtained as a brown oil.

LCMS: RT=1.147 min, MS cal.: 224.9, [M−H]⁻=223.8, 225.8

1H NMR (400 MHz, CHCl3-d) δ=7.02 (m, 1H), 3.88 (br s, 2H)

### General Procedure for Preparation of Intermediate 3:

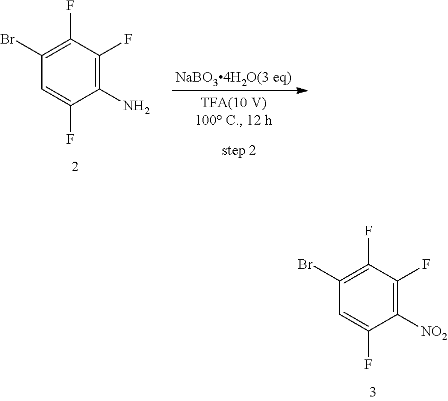 [SMILES: Nc1c(F)cc(Br)c(F)c1F ; O=[N+]([O-])c1c(F)cc(Br)c(F)c1F]

To a solution of 2 (17.5 g, 77.44 mmol, 1 eq) in TFA (175 mL) was added sodium; 3-oxidodioxaborirane, tetrahydrate (35.74 g, 232.31 mmol, 44.68 mL, 3 eq) at 20° C. The mixture was stirred at 100° C. for 12 hr. TLC showed Reactant 1 was consumed completely. The reaction mixture was concentrated under reduced pressure to remove TFA. The residue was diluted with H₂O (300 mL) and extracted with DCM (100 mL*3). The combined organic layers were washed with brine (100 mL*2), dried over Na₂SO₄, filtered and concentrated under reduced pressure to give a residue. The residue was purified by column chromatography (SiO₂, Petroleum ether/Ethyl acetate=1/0 to 10/1). 3 (8.5 g, 33.21 mmol, 43% yield) was obtained as an orange oil.

1H NMR (400 MHz, CHCl3-d) δ=7.43-7.38 (m, 1H)

### General Procedure for Preparation of Intermediate 4:

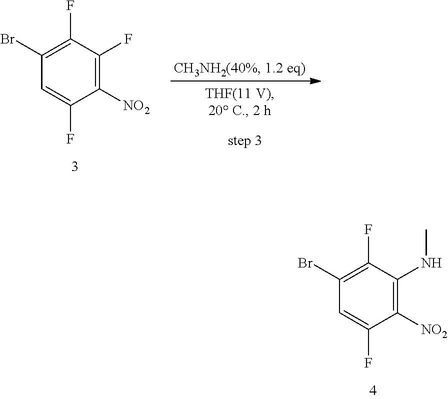

3 (8.5 g, 33.21 mmol, 1 eq) was charged to the round bottom flask, then THF (85 mL) was added at 20° C. under N₂. At 20° C. (inner temperature), methanamine (3.09 g, 39.85 mmol, 40% purity, 1.2 eq) in THF (8.5 mL) was added to the reaction mixture under N₂ atmosphere. After the addition, the mixture was degassed and purged with N₂ for 3 times, the mixture was stirred at 20° C. for 2 hr. LCMS (Rt=1.327) showed Reactant 1 was consumed completely and desired mass was detected. The reaction mixture was quenched by addition solution of 1 M FA to adjust pH=7, and then diluted with H₂O (200 mL) and extracted with EtOAc (100 mL*3). The combined organic layers were washed with brine (100 mL*2), dried over Na₂SO₄, filtered and concentrated under reduced pressure to give a residue. The residue was purified by column chromatography (SiO₂, Petroleum ether/Ethyl acetate=1/0 to 5/1). 4 (7.4 g, 27.71 mmol, 83% yield) was obtained as an orange solid.

LCMS: RT=1.327 min, MS cal.: 266.0, [M−H]⁻=264.8, 266.8

1H NMR (400 MHz, CHCl3-d) δ=6.68 (dd, J=5.2, 10.0 Hz, 1H), 6.56 (br d, J, 1H), 3.17 (d, J=6.8 Hz, 3H)

### General Procedure for Preparation of Intermediate 5:

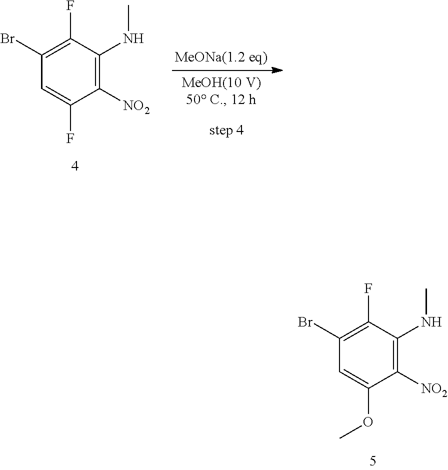

To a solution of 4 (3.4 g, 12.73 mmol, 1 eq) in MeOH (34 mL) was added CH3ONa (825.45 mg, 15.28 mmol, 1.2 eq) at 20° C. The mixture was stirred at 50° C. for 12 hr. LC-MS (Rt=1.286) showed Reactant 1 was consumed completely and desired mass was detected. The reaction mixture was diluted with H₂O (150 mL) and extracted with EtOAc (50 mL*3). The combined organic layers were washed with brine (50 mL*2), dried over Na₂SO₄, filtered and concentrated under reduced pressure to give a residue. The residue was purified by column chromatography (SiO₂, Petroleum ether/Ethyl acetate=100/1 to 20/1). 5 (2.9 g, 10.39 mmol, 82% yield) was obtained as an orange solid.

LCMS: RT=1.293 min, MS cal.: 264.0, [M−H]⁻=278.8, 280.8

1H NMR (400 MHz, CHCl3-d) δ=6.44 (d, J=4.9 Hz, 1H), 5.35-4.96 (m, 1H), 3.85 (s, 3H), 3.04 (d, J=4.6 Hz, 3H)

### General Procedure for Preparation of Intermediate 6:

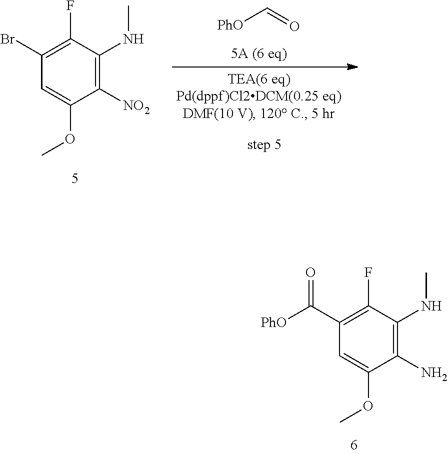 [SMILES: CNc1c(F)c(Br)cc(OC)c1[N+](=O)[O-] ; O=COc1ccccc1 ; CNc1c(N)c(OC)cc(C(=O)Oc2ccccc2)c1F]

To a solution of 5 (800 mg, 2.87 mmol, 1 eq) and phenyl formate (2.10 g, 17.20 mmol, 1.88 mL, 6 eq) in DMF (8 mL) was added TEA (1.74 g, 17.20 mmol, 2.39 mL, 6 eq) and Pd(dppf)Cl₂·CH₂Cl₂ (585.27 mg, 716.68 μmol, 0.25 eq) at 20° C. under N₂. The mixture was stirred at 120° C. for 5 hr. LC-MS (Rt=1.220) showed Reactant 1 was consumed completely and desired mass was detected. The reaction mixture was filtered and the filter cake was washed with EtOAc (20 mL). The filtrate was diluted with H₂O (50 mL) and extracted with EtOAc (15 mL*3). The combined organic layers were washed with brine (20 mL*2), dried over Na₂SO₄, filtered and concentrated under reduced pressure to give a residue. The residue was purified by column chromatography (SiO₂, Petroleum ether/Ethyl acetate=10/1 to 3/1). 6 (190 mg, 654.52 μmol, 23% yield) was obtained as a brown oil.

LCMS: RT=1.214 min, MS cal.: 290.1, [M+H]⁺=290.9

1H NMR (400 MHz, DMSO-d6) δ=7.49-7.39 (m, 2H), 7.32-7.25 (m, 1H), 7.24-7.18 (m, 2H), 7.02 (d, J=6.2 Hz, 1H), 5.59 (br s, 2H), 4.22 (br d, J=1.9 Hz, 1H), 3.81 (s, 3H), 2.71 (d, J=2.0 Hz, 3H)

### General Procedure for Preparation of Intermediate 7:

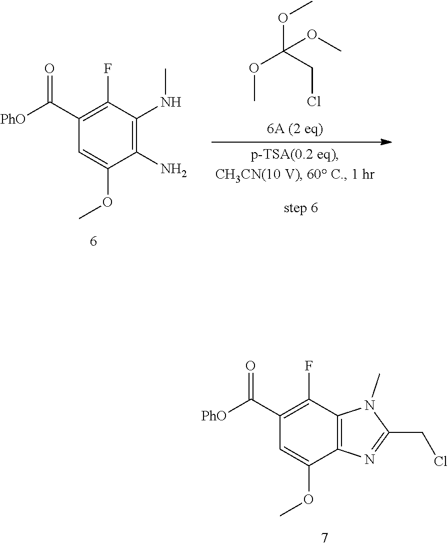

To a solution of 6 (350 mg, 1.21 mmol, 1 eq) and 2-chloro-1,1,1-trimethoxy-ethane (372.78 mg, 2.41 mmol, 325.00 μL, 2 eq) in ACN (3 mL) was added TosOH (41.52 mg, 241.14 μmol, 0.2 eq) at 20° C. The mixture was stirred at 60° C. for 1 hr. LC-MS (Rt=1.263) showed Reactant 1 was consumed completely and desired mass was detected. The residue was diluted with H₂O (10 mL) and extracted with EtOAc (5 mL*3). The combined organic layers were washed with saturated sodium bicarbonate solution (5 mL*2), dried over Na₂SO₄, filtered, and concentrated under reduced pressure to give a residue. 7 (330 mg, 946.22 μmol, 78% yield) was obtained as a white solid.

LCMS: RT=1.263 min, MS cal.: 348.0/350.0, [M+H]⁺=348.9/350.9

1H NMR (400 MHz, DMSO-d6) δ=7.54-7.43 (m, 2H), 7.34-7.27 (m, 3H), 7.21 (d, J=4.6 Hz, 1H), 5.12 (s, 2H), 4.05 (s, 3H), 3.97 (s, 3H)

### General Procedure for Preparation of Intermediate 8:

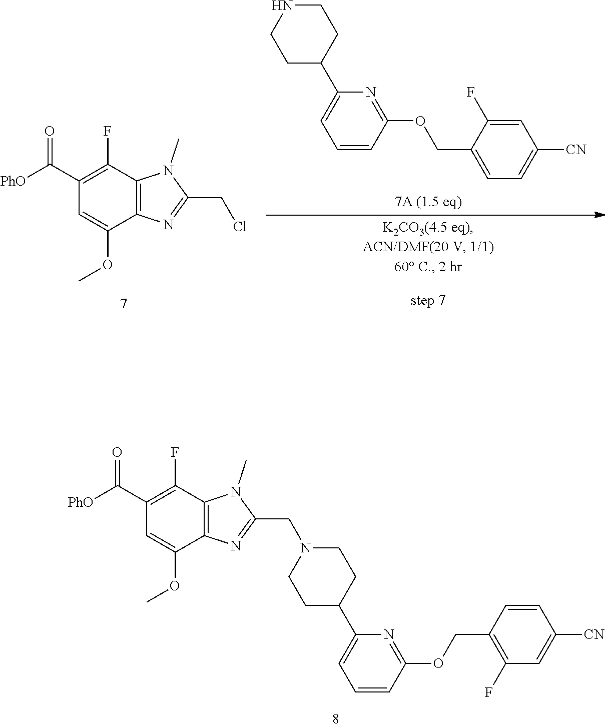 [SMILES: COc1cc(C(=O)Oc2ccccc2)c(F)c2c1nc(CCl)n2C ; N#Cc1ccc(COc2cccc(C3CCNCC3)n2)c(F)c1 ; COc1cc(C(=O)Oc2ccccc2)c(F)c2c1nc(CN1CCC(c3cccc(OCc4ccc(C#N)cc4F)n3)CC1)n2C]

To a solution of 7A (141.00 mg, 344.08 μmol, 1.5 eq, 2.7HCl) in ACN (0.8 mL) and DMF (0.8 mL) was added K₂CO₃ (95.11 mg, 688.16 μmol, 3 eq) at 15° C. and stirred for 0.15 hr. The mixture was added 7 (80 mg, 229.39 μmol, 1 eq) and K₂CO₃ (47.56 mg, 344.08 μmol, 1.5 eq) at 15° C. and stirred at 60° C. for 1.75 hr. LC-MS (Rt=0.492) showed it was consumed completely and desired mass was detected. The reaction mixture was diluted with H₂O (5 mL) and extracted with EtOAc (2 mL*3). The combined organic layers were washed with brine (2 mL*2), dried over Na₂SO₄, filtered and concentrated under reduced pressure to give a crude product. The residue was purified by column chromatography (SiO₂, Petroleum ether/Ethyl acetate=9/1 to 1/1). 8 (100 mg, 160.35 μmol, 70% yield) was obtained as a white solid.

LCMS: RT=0.477 min, MS cal.: 623.2/624.2, [M+H]⁺=624.4/625.3

1H NMR (400 MHz, DMSO-d6) δ=7.87 (d, J=9.9 Hz, 1H), 7.69 (br s, 2H), 7.64 (t, J=7.7 Hz, 1H), 7.53-7.45 (m, 2H), 7.38-7.25 (m, 3H), 7.17 (br d, J=4.1 Hz, 1H), 6.87 (d, J=7.3 Hz, 1H), 6.71 (d, J=8.2 Hz, 1H), 5.45 (s, 2H), 4.08 (s, 3H), 3.96 (s, 3H), 3.83 (br s, 2H), 2.91 (br s, 2H), 2.56 (br d, J=9.6 Hz, 1H), 2.26-2.11 (m, 2H), 1.81-1.60 (m, 4H)

### General Procedure for Preparation of Compound 303 (9):

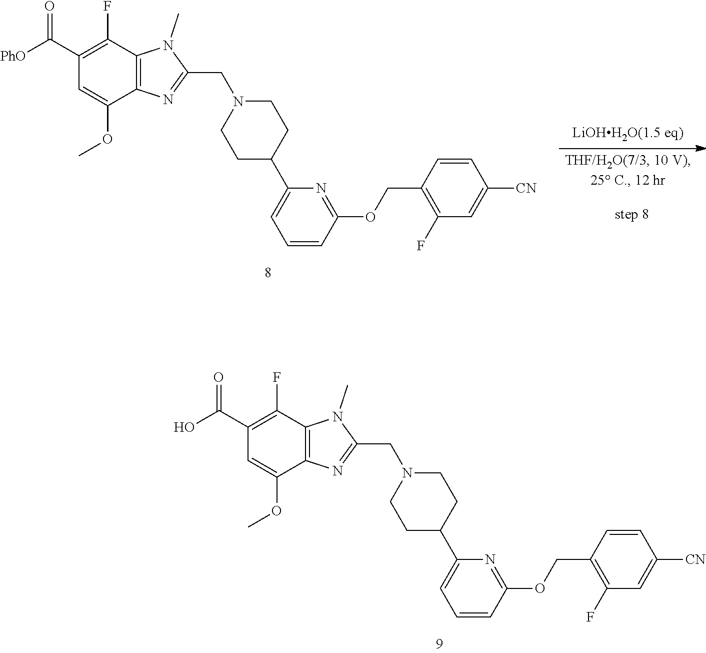 [SMILES: COc1cc(C(=O)Oc2ccccc2)c(F)c2c1nc(CN1CCC(c3cccc(OCc4ccc(C#N)cc4F)n3)CC1)n2C ; COc1cc(C(=O)O)c(F)c2c1nc(CN1CCC(c3cccc(OCc4ccc(C#N)cc4F)n3)CC1)n2C]

To a solution of 8 (111.05 mg, 178.07 μmol, 1 eq) in THF (0.7 mL) and H₂O (0.3 mL) was added LiOH·H₂O (11.21 mg, 267.10 μmol, 1.5 eq) at 15° C., the mixture was stirred at 25° C. for 12 hr. LCMS showed Compound 2 was consumed completely. The reaction mixture was filtered, purified by prep-HPLC (neutral condition; column: Waters Xbridge Prep OBD C18 150*40 mm*10 um; mobile phase: [H₂O (10 mM NH₄HCO₃)-ACN]; gradient: 25%-55% B over 8.0 min) to give 2-((4-(6-((4-Cyano-2-fluorobenzyl)oxy)pyridin-2-yl)piperidin-1-yl)methyl)-7-fluoro-4-methoxy-1-methyl-1H-benzo[d]imidazole-6-carboxylic acid (28.68 mg, 52.38 μmol, 29% yield) as a white solid.

LCMS: RT=0.399 min, MS cal.: 547.56, [M+H]⁺=548.3

1H NMR (400 MHz, MeOH-d4) δ=7.69 (t, J=7.6 Hz, 1H), 7.63-7.52 (m, 3H), 7.07 (d, J=4.8 Hz, 1H), 6.85 (d, J=7.2 Hz, 1H), 6.68 (d, J=8.2 Hz, 1H), 5.53 (s, 2H), 4.15 (s, 3H), 3.99 (s, 3H), 3.86 (s, 2H), 3.01 (br d, J=11.4 Hz, 2H), 2.68-2.56 (m, 1H), 2.36-2.25 (m, 2H), 1.90-1.78 (m, 4H)

### Example A77—Preparation of Compound 304. 2-(4-(6-((4-Cyano-2-fluorobenzyl)oxy)pyridin-2-yl)-2,5-difluorobenzyl)-4-methoxy-1-methyl-1H-benzo[d]imidazole-6-carboxylic acid (Compound 304) (5)

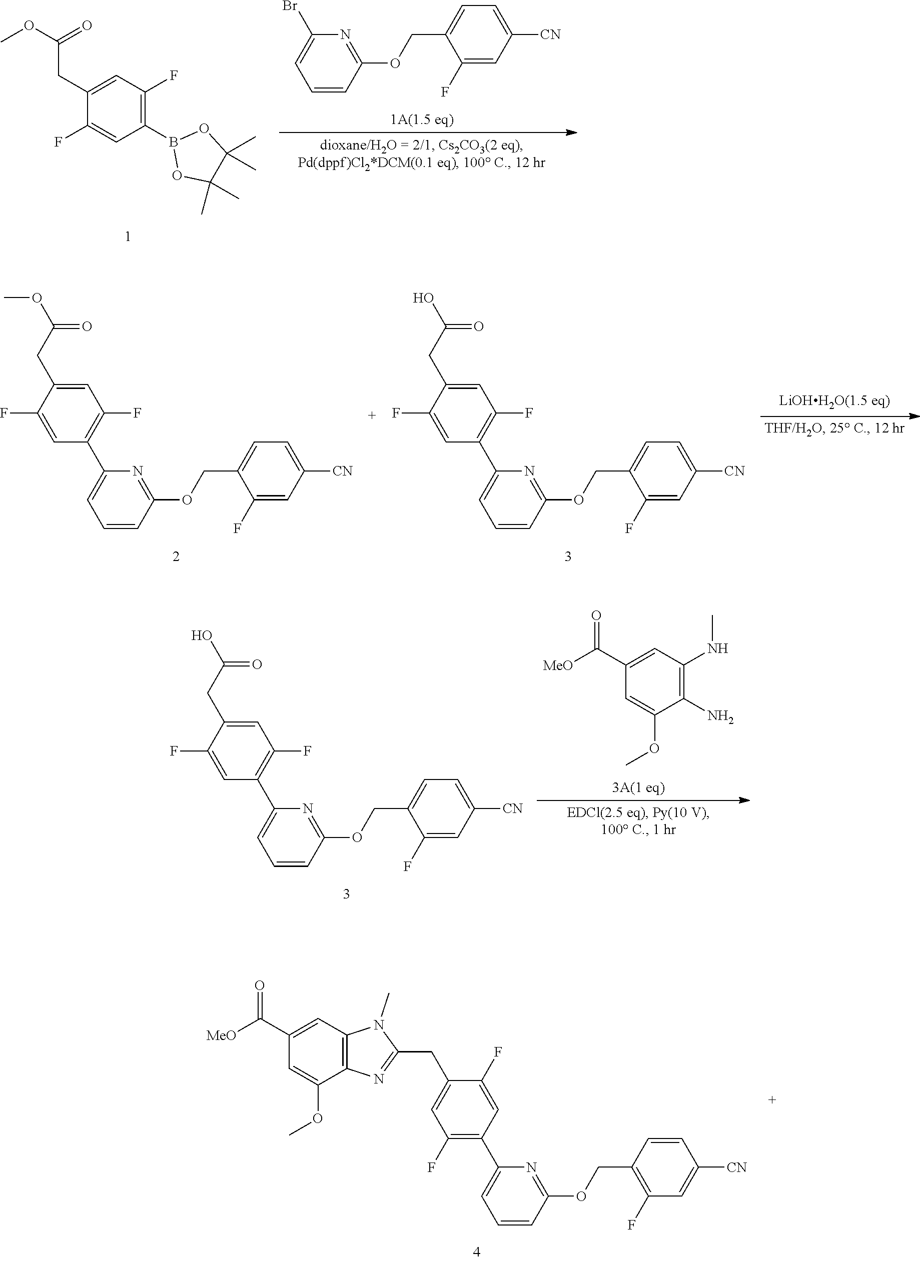

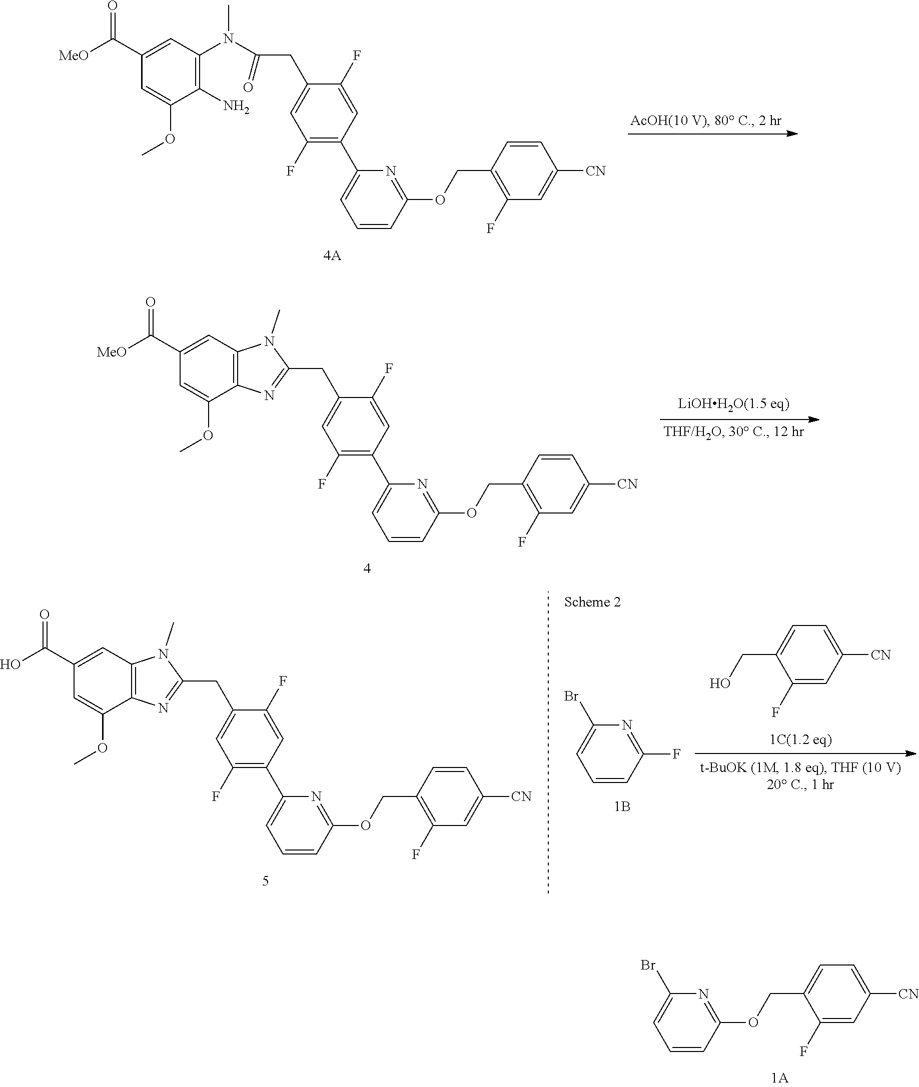 [SMILES: COC(=O)c1cc(OC)c(N)c(N(C)C(=O)Cc2cc(F)c(-c3cccc(OCc4ccc(C#N)cc4F)n3)cc2F)c1 ; COC(=O)c1cc(OC)c2nc(Cc3cc(F)c(-c4cccc(OCc5ccc(C#N)cc5F)n4)cc3F)n(C)c2c1 ; COc1cc(C(=O)O)cc2c1nc(Cc1cc(F)c(-c3cccc(OCc4ccc(C#N)cc4F)n3)cc1F)n2C ; Fc1cccc(Br)n1 ; N#Cc1ccc(CO)c(F)c1 ; N#Cc1ccc(COc2cccc(Br)n2)c(F)c1]

### General Procedure for Preparation of Intermediate 1A:

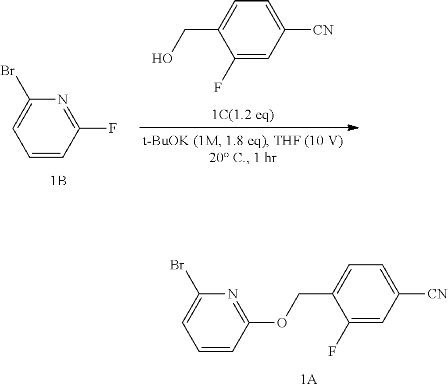 [SMILES: Fc1cccc(Br)n1 ; N#Cc1ccc(CO)c(F)c1 ; N#Cc1ccc(COc2cccc(Br)n2)c(F)c1]

To a solution of 1B (5 g, 28.41 mmol, 1 eq) in THF (50 mL) was added t-BuOK (1 M, 51.14 mL, 1.8 eq) and 1C (5.15 g, 34.09 mmol, 1.2 eq). The mixture was stirred at 20° C. for 1 hr. LCMS showed 1B was consumed completely and desired mass was detected. The reaction mixture was quenched with 20 mL sat. NH₄Cl, then 100 mL H₂O was added to the mixture and extracted with EtOAc (100 mL×3), the combined organic phase was washed with 100 mL brine, dried over anhydrous Na₂SO₄, filtered and concentrated under reduced pressure to give a residue. The residue was purified by column chromatography (SiO₂, Petroleum ether/Ethyl acetate=1/0 to 0/1). 1A (8.45 g, 27.51 mmol, 48.42% yield) was obtained as a white solid.

LCMS: RT=1.465 min, MS cal.: 306.0, [M+H]⁺=307.0

¹H NMR (400 MHz, CHCl3-d) δ=7.67 (t, J=7.6 Hz, 1H), 7.53-7.44 (m, 2H), 7.40 (dd, J=1.2, 9.3 Hz, 1H), 7.13 (d, J=7.2 Hz, 1H), 6.78 (d, J=8.0 Hz, 1H), 5.49 (s, 2H)

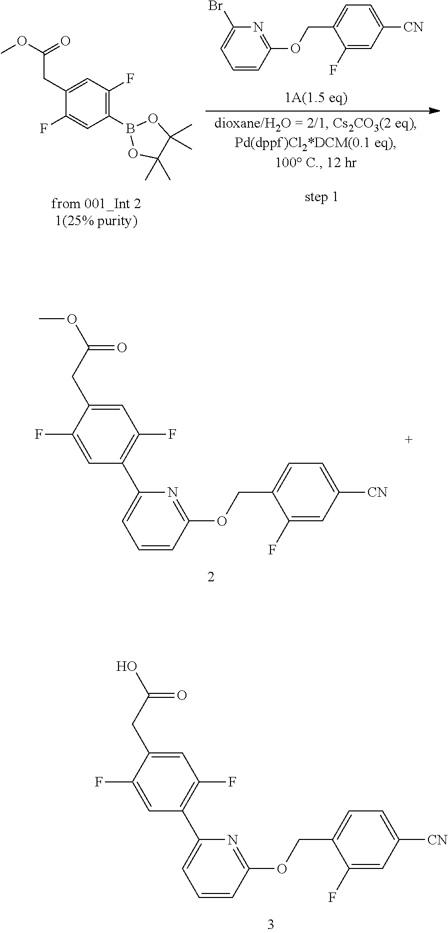

To a solution of 1 (0.7 g, 560.69 μmol, 1 eq, 25% purity, synthesized from Int 2) and 1A (258.30 mg, 841.03 μmol, 1.5 eq) in dioxane (4.9 mL) and H₂O (2.1 mL) was added Cs₂CO₃ (365.37 mg, 1.12 mmol, 2 eq) and Pd(dppf)Cl₂·DCM (45.79 mg, 56.07 μmol, 0.1 eq) at 25° C. The mixture was stirred at 100° C. for 12 hr. LC-MS showed 1 was consumed completely and desired mass was detected. 3 was also detected. The reaction mixture was diluted with H₂O 30 mL and extracted with EtOAc (10 mL*3). The combined organic layers were washed with brine 20 mL, dried over Na₂SO₄, filtered and concentrated under reduced pressure to give a residue. The residue was purified by column chromatography (SiO₂, Petroleum ether/Ethyl acetate=10/1 to I/O). 2 (68 mg, 164.90 μmol, 29.41% yield) was obtained as a white solid and 3 (320 mg, crude) was obtained as a yellow solid.

LCMS (Compound 2): RT=1.542 min, MS cal.: 412.1, [M+H]⁺=412.9

LCMS (Compound 3): RT=1.048 min, MS cal.: 398.1, [M+H]⁺=399.0

¹H NMR (400 MHz, CHCl3-d) (2) δ=7.78-7.61 (m, 3H), 7.53 (dd, J=1.2, 7.4 Hz, 1H), 7.47 (dd, J=1.2, 7.9 Hz, 1H), 7.42 (dd, J=1.2, 9.3 Hz, 1H), 7.10 (dd, J=6.0, 11.3 Hz, 1H), 6.84 (d, J=8.0 Hz, 1H), 5.59 (s, 2H), 3.76 (s, 3H), 3.71 (s, 2H)

¹H NMR (400 MHz, CHCl3-d) (3) δ=7.74-7.63 (m, 3H), 7.52 (d, J=6.8 Hz, 1H), 7.47 (d, J=8.0 Hz, 1H), 7.41 (d, J=9.2 Hz, 1H), 7.11 (dd, J=6.0, 11.1 Hz, 1H), 6.83 (d, J=8.0 Hz, 1H), 5.59 (s, 2H), 3.73 (s, 2H), 1.30-1.23 (m, 208H), 1.31-1.18 (m, 1H)

### General Procedure for Preparation of Intermediate 3:

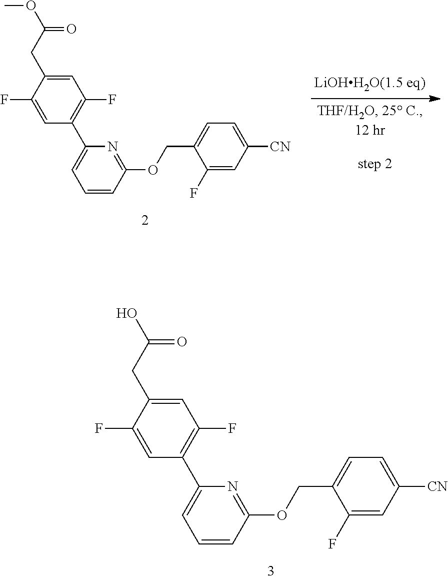 [SMILES: COC(=O)Cc1cc(F)c(-c2cccc(OCc3ccc(C#N)cc3F)n2)cc1F ; N#Cc1ccc(COc2cccc(-c3cc(F)c(CC(=O)O)cc3F)n2)c(F)c1]

To a solution of 2 (68 mg, 164.90 μmol, 1 eq) in THF (0.7 mL) was added LiOH·H₂O (10.38 mg, 247.36 μmol, 1.5 eq) in H₂O (0.3 mL). The mixture was stirred at 25° C. for 12 hr. LC-MS showed 2 was consumed completely and desired mass was detected. The reaction mixture was added with 1M FA to adjust pH=3-4. Then the mixture was diluted with H₂O 15 mL and extracted with EtOAc (10 mL*3). The combined organic layers were washed with brine 15 mL, dried over anhydrous Na₂SO₄, filtered, and concentrated under reduced pressure to give a residue. The crude product 3 (60 mg, crude, a yellow solid) was used into the next step without further purification.

LCMS: RT=1.047 min, MS cal.: 398.1, [M+H]⁺=398.9

¹H NMR (400 MHz, CHCl3-d) δ=7.76-7.64 (m, 3H), 7.55-7.40 (m, 3H), 7.11 (dd, J=6.0, 11.1 Hz, 1H), 6.84 (d, J=8.0 Hz, 1H), 5.59 (s, 2H), 3.76 (s, 2H)

### General Procedure for Preparation of Intermediate 4 & 4A:

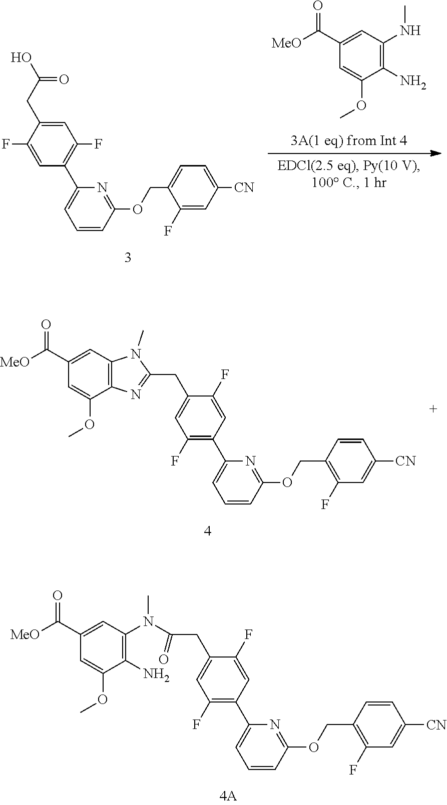 [SMILES: N#Cc1ccc(COc2cccc(-c3cc(F)c(CC(=O)O)cc3F)n2)c(F)c1 ; CNc1cc(C(=O)OC)cc(OC)c1N ; COC(=O)c1cc(OC)c2nc(Cc3cc(F)c(-c4cccc(OCc5ccc(C#N)cc5F)n4)cc3F)n(C)c2c1 ; COC(=O)c1cc(OC)c(N)c(N(C)C(=O)Cc2cc(F)c(-c3cccc(OCc4ccc(C#N)cc4F)n3)cc2F)c1]

To a solution of 3 (270 mg, 155.90 μmol, 1 eq) and 3A (32.77 mg, 155.90 μmol, 1 eq) in Py (2.7 mL) was added EDCI (74.71 mg, 389.75 μmol, 2.5 eq) at 20° C. The mixture was stirred at 100° C. for 1 hr. LC-MS showed 3 was consumed completely and 4 & 4A was detected. The reaction mixture was adjusted to pH=3 with 1M FA. Then 30 mL H₂O was added to the mixture and extracted with EtOAc (30 mL*3). The combined organic phase was washed with brine (20 mL*2), dried with anhydrous Na₂SO₄, filtered and concentrated in vacuum. The crude product 4 & 4A (390 mg, crude, a yellow solid, ratio: 1:3 in LCMS) was used into the next step without further purification.

LCMS (4): RT=1.862 min, MS cal: 572.2, [M+H]⁺=573.4

LCMS (4A): RT=2.141 min, MS cal: 590.2, [M+H]⁺=591.4

### General Procedure for Preparation of Intermediate 4:

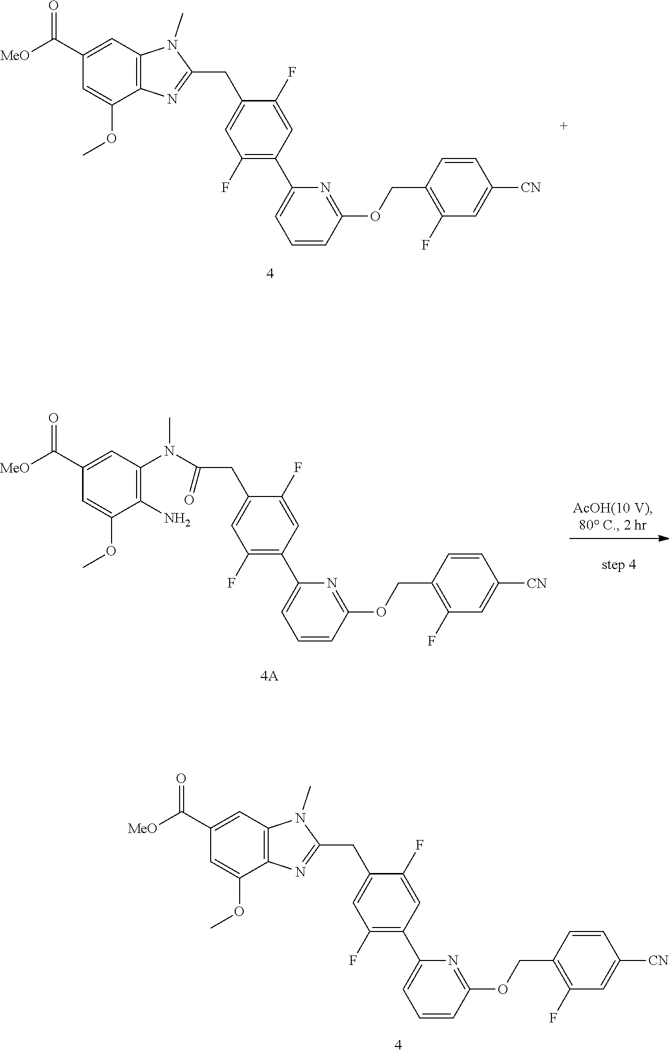 [SMILES: COC(=O)c1cc(OC)c2nc(Cc3cc(F)c(-c4cccc(OCc5ccc(C#N)cc5F)n4)cc3F)n(C)c2c1 ; COC(=O)c1cc(OC)c(N)c(N(C)C(=O)Cc2cc(F)c(-c3cccc(OCc4ccc(C#N)cc4F)n3)cc2F)c1 ; COC(=O)c1cc(OC)c2nc(Cc3cc(F)c(-c4cccc(OCc5ccc(C#N)cc5F)n4)cc3F)n(C)c2c1]

4 & 4A (390 mg, crude, ratio: 1:3 in LCMS) was dissolved in CH₃COOH (4 mL) at 25° C. The mixture was stirred at 80° C. for 2 hr. LC-MS showed 4 & 4A was consumed completely and desired mass was detected. The reaction mixture was concentrated under reduced pressure to remove CH₃COOH. The reaction mixture was diluted with sat. NaHCO₃25 mL and extracted with EtOAc (20 mL*3). The combined organic layers were washed with brine 20 mL dried over anhydrous Na₂SO₄, filtered and concentrated under reduced pressure to give a residue. The residue was purified by column chromatography (SiO₂, Petroleum ether/Ethyl acetate=2/1 to 1/4). 4 (100 mg, 174.66 μmol, 26.45% yield) was obtained as a white solid.

LCMS: RT=1.872 min, MS cal.: 590.2, [M+H]⁺=573.3

¹H NMR (400 MHz, CHCl3-d) δ=7.78-7.63 (m, 4H), 7.54-7.40 (m, 4H), 6.84 (d, J=8.0 Hz, 1H), 5.59 (s, 2H), 5.31 (s, 1H), 4.10 (s, 3H), 3.97 (s, 3H), 3.78 (br s, 2H)

### General Procedure for Preparation of Compound 304 (5):

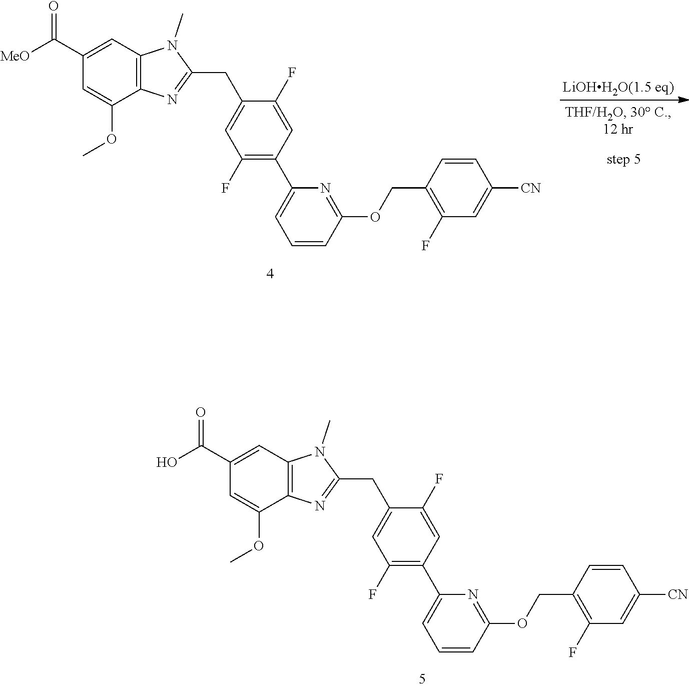 [SMILES: COC(=O)c1cc(OC)c2nc(Cc3cc(F)c(-c4cccc(OCc5ccc(C#N)cc5F)n4)cc3F)n(C)c2c1 ; COc1cc(C(=O)O)cc2c1nc(Cc1cc(F)c(-c3cccc(OCc4ccc(C#N)cc4F)n3)cc1F)n2C]

To a solution of 4 (90 mg, 157.20 μmol, 1 eq) in THF (1.4 mL) was added LiOH·H₂O (9.89 mg, 235.79 μmol, 1.5 eq) in H₂O (0.6 mL). The mixture was stirred at 30° C. for 12 hr. LC-MS showed ˜50% of 4 remained. Several new peaks were shown on LC-MS and ˜50% of desired Compound was detected. The residue was purified by prep-HPLC (column: Waters Xbridge Prep OBD C18 150*40 mm*10 um; mobile phase: [H₂O (10 mM NH₄HCO₃)-ACN]; gradient: 20%-65% B over 8.0 min). 2-(4-(6-((4-Cyano-2-fluorobenzyl)oxy)pyridin-2-yl)-2,5-difluorobenzyl)-4-methoxy-1-methyl-1H-benzo[d]imidazole-6-carboxylic acid (21.84 mg, 39.10 μmol, 24.88% yield, 98.31% purity) was obtained as a white solid.

LCMS: RT=2.624 min, MS cal.: 558.2, [M+H]⁺=559.2

HPLC: RT=12.241 min, purity: 98.31%

¹H NMR (400 MHz, DMSO-d₆) δ=7.97-7.83 (m, 2H), 7.79-7.67 (m, 4H), 7.55-7.49 (m, 1H), 7.33 (dd, J=6.0, 11.4 Hz, 1H), 7.27 (d, J=1.2 Hz, 1H), 6.98 (d, J=8.0 Hz, 1H), 5.60 (s, 2H), 4.37 (s, 2H), 3.89 (s, 3H), 3.84 (s, 3H)

### Example A78—Preparation of Compound 341. 2-((4-(6-((4-Chloro-2-fluorobenzyl)oxy)pyridin-2-yl)piperidin-1-yl)methyl)-7-hydroxy-1-methyl-1H-benzo[d]imidazole-6-carboxylic acid (Compound 341) (6)

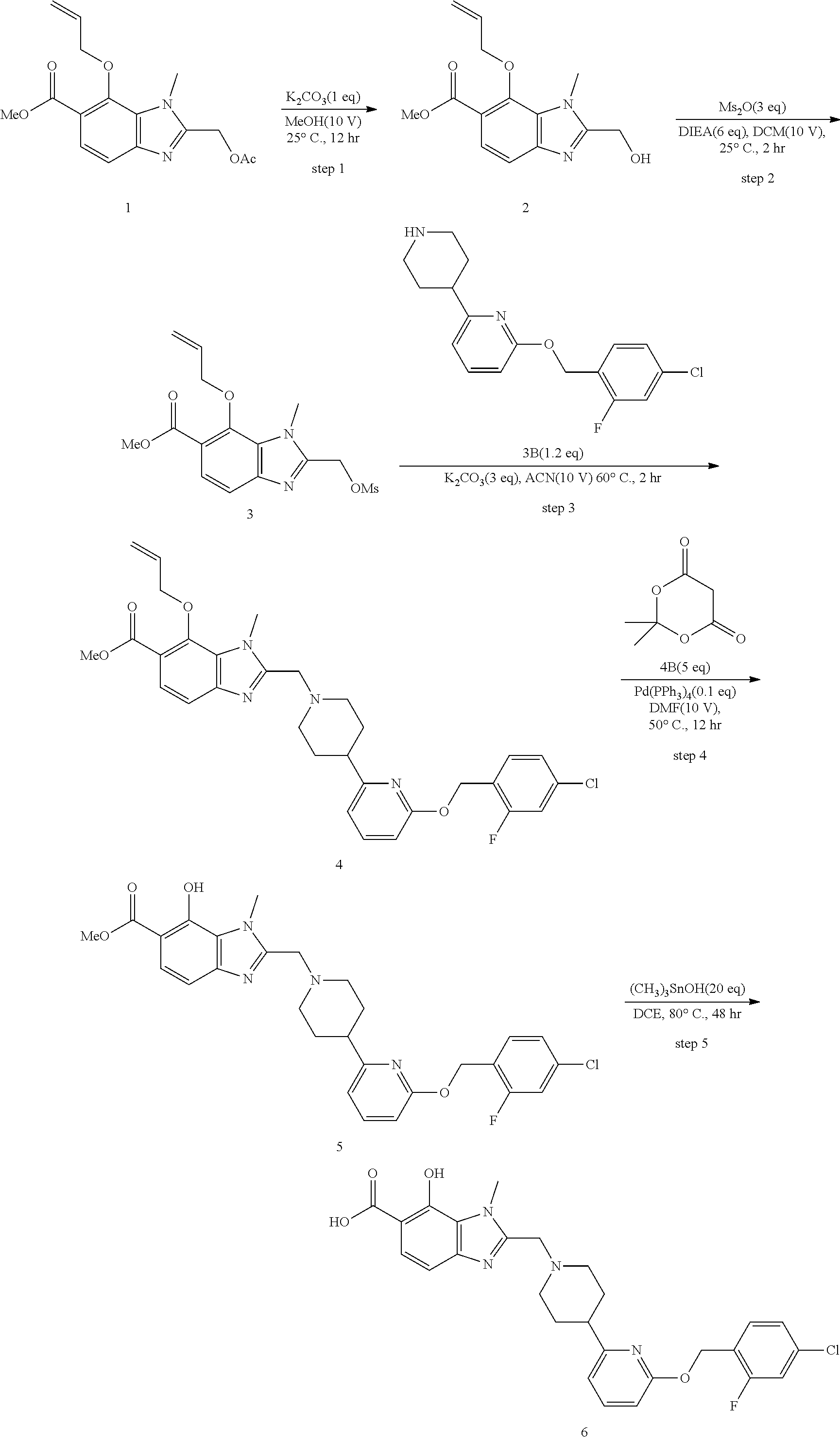

### General Procedure for Preparation of Intermediate 2:

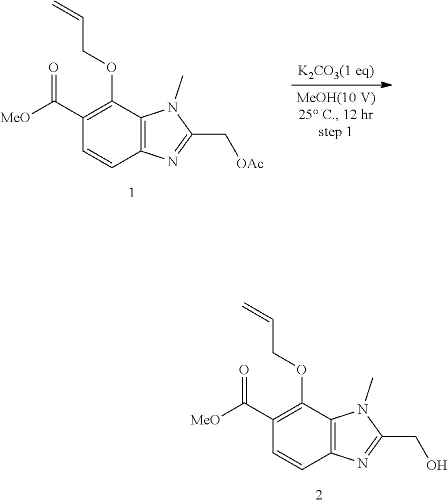 [SMILES: C=CCOc1c(C(=O)OC)ccc2nc(COC(C)=O)n(C)c12 ; C=CCOc1c(C(=O)OC)ccc2nc(CO)n(C)c12]

A mixture of 1 (200 mg, 628.29 μmol, 1 eq), K₂CO₃ (86.83 mg, 628.29 μmol, 1 eq) in MeOH (2 mL) was degassed and purged with N₂ for 3 times, and then the mixture was stirred at 25° C. for 12 hr under N₂ atmosphere. TLC (Petroleum ether/Ethyl acetate=0/1, R_{f}=0.24) indicated 1 was consumed completely and one new spot formed. The reaction mixture was diluted with H₂O 10 mL and extracted with DCM (10 mL*3). The combined organic layers were dried over Na₂SO₄, filtered, and concentrated under reduced pressure to give a residue. 2 (170 mg, crude) was obtained as a yellow solid.

### General Procedure for Preparation of Intermediate 3:

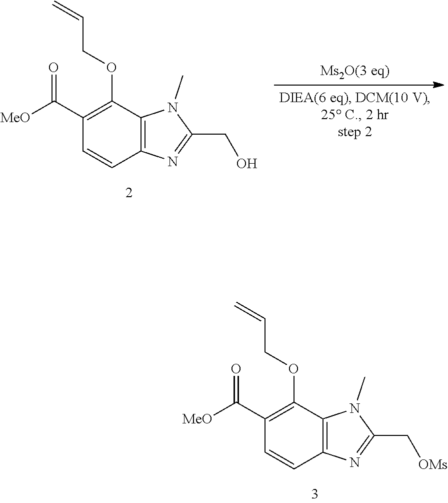 [SMILES: C=CCOc1c(C(=O)OC)ccc2nc(CO)n(C)c12 ; C=CCOc1c(C(=O)OC)ccc2nc(COS(C)(=O)=O)n(C)c12]

A mixture of 2 (170 mg, 615.30 μmol, 1 eq), Ms₂O (321.55 mg, 1.85 mmol, 3 eq), DIEA (477.14 mg, 3.69 mmol, 643.04 μL, 6 eq) in DCM (1.7 mL) was degassed and purged with N₂ for 3 times, and then the mixture was stirred at 25° C. for 2 hr under N₂ atmosphere. LCMS (RT=1.045 min) showed 2 was consumed completely and one main peak with desired mass was detected. The reaction mixture was added HCOOH at 25° C. until pH=7-8, and then diluted with H₂O 10 mL and extracted with DCM 10 mL*4. The combined organic layers were dried over Na₂SO₄, filtered, and concentrated under reduced pressure to give a residue. 3 (220 mg, crude) was obtained as a yellow solid.

### General Procedure for Preparation of Intermediate 4:

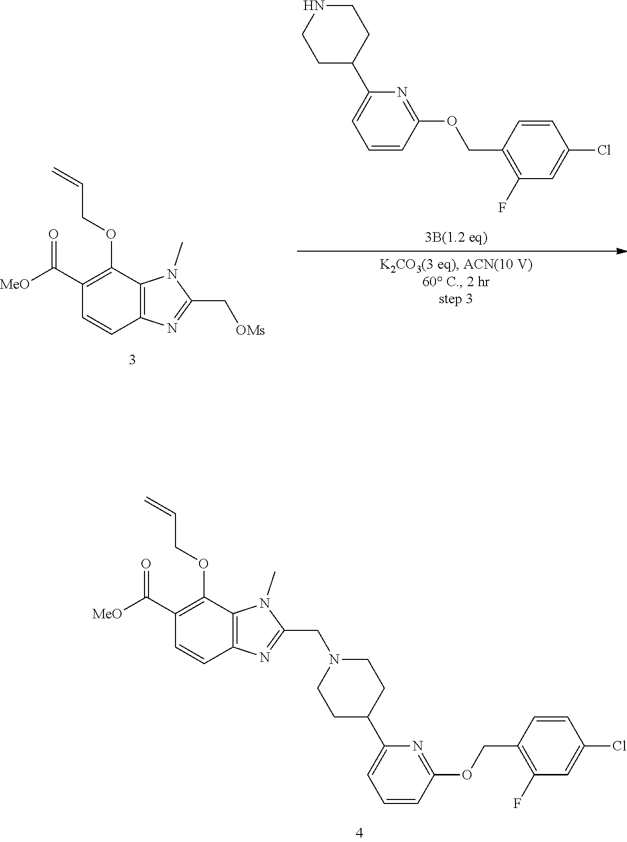 [SMILES: C=CCOc1c(C(=O)OC)ccc2nc(COS(C)(=O)=O)n(C)c12 ; Fc1cc(Cl)ccc1COc1cccc(C2CCNCC2)n1 ; C=CCOc1c(C(=O)OC)ccc2nc(CN3CCC(c4cccc(OCc5ccc(Cl)cc5F)n4)CC3)n(C)c12]

A mixture of 3 (220 mg, 620.81 μmol, 1 eq), 3B (238.98 mg, 744.97 μmol, 1.2 eq), K₂CO₃ (257.40 mg, 1.86 mmol, 3 eq) in ACN (2.2 mL) was degassed and purged with N₂ for 3 times, and then the mixture was stirred at 60° C. for 2 hr under N₂ atmosphere. LCMS (RT=1.728 min) showed 3 was consumed completely and one main peak with desired mass was detected. The reaction mixture was diluted with H₂O (10 mL) and extracted with DCM (10 mL*3). The combined organic layers were dried over Na₂SO₄, filtered and concentrated under reduced pressure to give a residue. The residue was purified by column chromatography (SiO₂, Petroleum ether/Ethyl acetate=100/1 to 0/1). 4 (210 mg, 362.66 μmol, 58% yield) was obtained as a white solid.

### General Procedure for Preparation of Intermediate 5:

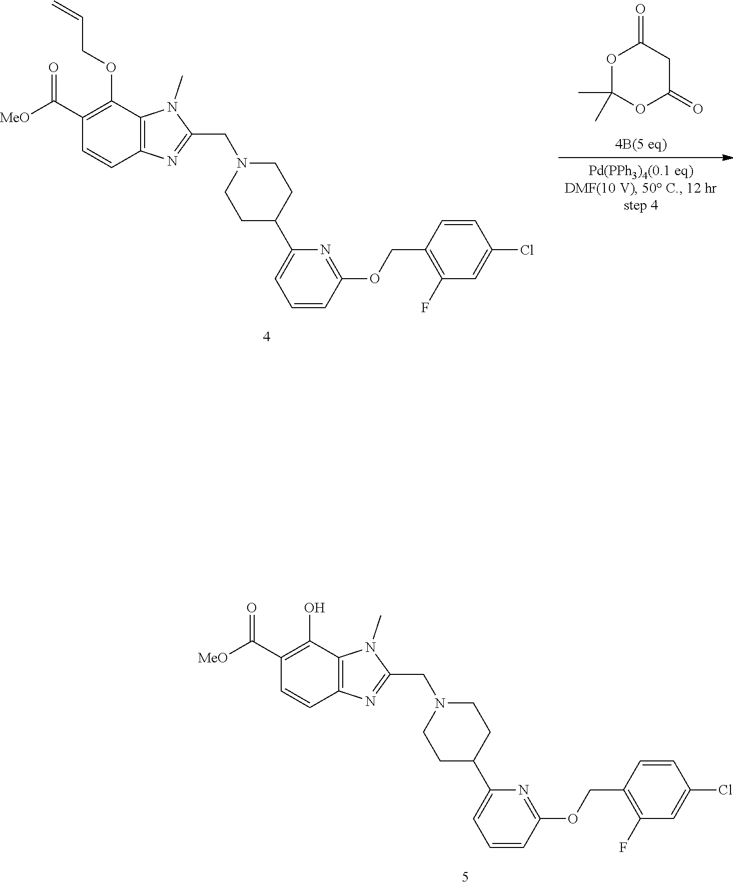

A mixture of 4 (160 mg, 276.31 μmol, 1 eq), Pd(PPh₃)₄ (10.19 mg, 27.63 mol, 0.1 eq), 4B (199.12 mg, 1.38 mmol, 5 eq) in DMF (1.5 mL) was degassed and purged with N₂ for 3 times, and then the mixture was stirred at 50° C. for 12 hr under N₂ atmosphere. LCMS (RT=1.778 min) showed 4 was consumed completely and one main peak with desired mass was detected. The reaction mixture was filtered. The filtrate was purified by prep-HPLC (column: Waters Xbridge Prep OBD C₁₈ 150*40 mm*10 um; mobile phase: [H₂O (10 mM NH₄HCO₃)-ACN]; gradient: 75%-95% B over 8.0 min). 5 (80 mg, 148.42 μmol, 53% yield) was obtained as a white solid.

LCMS: RT=1.777 min, MS cal.: 538.2, [M+H]⁺=539.1

¹H NMR (400 MHz, CHCl3-d) δ=11.66 (s, 1H), 7.66 (d, J=8.8 Hz, 1H), 7.50 (t, J=7.6 Hz, 1H), 7.45 (t, J=8.0 Hz, 1H), 7.19 (d, J=8.8 Hz, 1H), 7.15-7.07 (m, 2H), 6.74 (d, J=7.6 Hz, 1H), 6.61 (d, J=8.0 Hz, 1H), 5.41 (s, 2H), 4.26 (s, 3H), 3.98 (s, 3H), 3.90-3.78 (m, 2H), 3.21-2.89 (m, 2H), 2.72-2.56 (m, 1H), 2.44-2.13 (m, 2H), 1.98-1.78 (m, 4H)

### General Procedure for Preparation of Compound 341 (6):

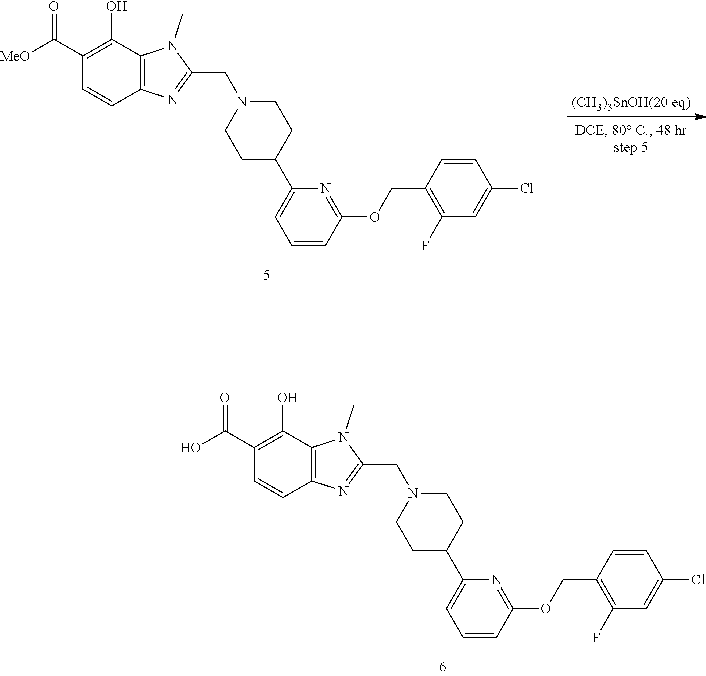 [SMILES: COC(=O)c1ccc2nc(CN3CCC(c4cccc(OCc5ccc(Cl)cc5F)n4)CC3)n(C)c2c1O ; Cn1c(CN2CCC(c3cccc(OCc4ccc(Cl)cc4F)n3)CC2)nc2ccc(C(=O)O)c(O)c21]

DCE (0.6 mL) was charged to the three-necked round bottom flask, then 5 (60 mg, 111.32 μmol, 1 eq) was added to the mixture at 25° C. At 25° C., (CH₃)₃SnOH (402.57 mg, 2.23 mmol, 20 eq) was added to the reaction mixture at 25° C. After the addition, the mixture was stirred at 80° C. for 48 hr. LCMS (RT=1.583 min) showed 5 was consumed completely and one main peak with desired mass was detected. The reaction mixture was added saturated KF solution (3 ml) at 25° C., the mixture was diluted with H₂O 5 mL and extracted with DCM (10 mL*4). The combined organic layers were dried over Na₂SO₄, filtered, and concentrated under reduced pressure to give a residue. The residue was purified by prep-HPLC (column: Phenomenex Luna C₁₈ 100*30 mm*3 um; mobile phase: [H₂O (0.2% FA)-ACN]; gradient: 30%-60% B over 8.0 min). 2-((4-(6-((4-Chloro-2-fluorobenzyl)oxy)pyridin-2-yl)piperidin-1-yl)methyl)-7-hydroxy-1-methyl-1H-benzo[d]imidazole-6-carboxylic acid (25.57 mg, 48.71 μmol, 43% yield) was obtained as a white solid.

LCMS: RT=2.808 min, MS cal.: 525.2, [M+H]⁺=524.2

HPLC: RT=11.739 min, purity: 96.98%

¹H NMR (400 MHz, MeOH-d4) δ=7.77 (d, J=8.4 Hz, 1H), 7.65-7.58 (m, 1H), 7.50 (t, J=8.0 Hz, 1H), 7.26-7.16 (m, 2H), 7.06 (d, J=8.4 Hz, 1H), 6.87 (d, J=7.6 Hz, 1H), 6.67 (d, J=8.0 Hz, 1H), 5.42 (s, 2H), 4.24 (s, 3H), 4.20 (s, 2H), 3.38 (s, 2H), 2.90-2.69 (m, 3H), 2.10-1.97 (m, 4H)

### Example A79—Preparation of Compound 305. 2-((4-(6-((4-(Difluoromethyl)-2-fluorobenzyl)oxy)pyridin-2-yl)piperidin-1-yl)methyl)-4-methoxy-1-methyl-1H-benzo[d]imidazole-6-carboxylic acid (Compound 305) (3)

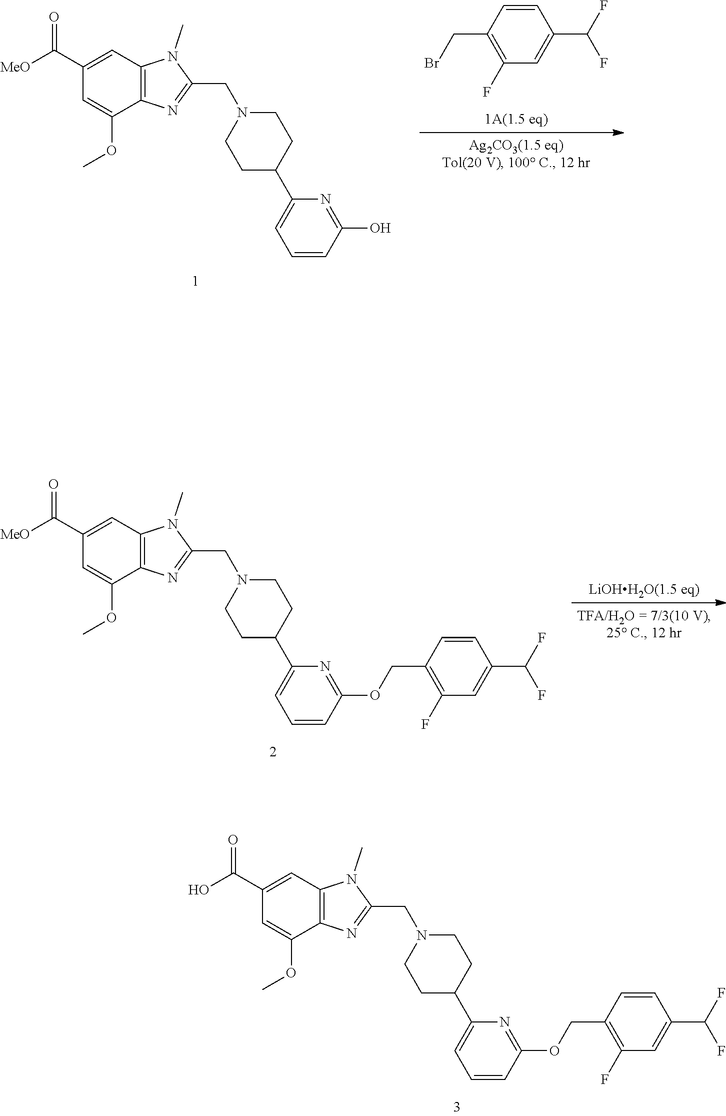 [SMILES: COC(=O)c1cc(OC)c2nc(CN3CCC(c4cccc(O)n4)CC3)n(C)c2c1 ; Fc1cc(C(F)F)ccc1CBr ; COC(=O)c1cc(OC)c2nc(CN3CCC(c4cccc(OCc5ccc(C(F)F)cc5F)n4)CC3)n(C)c2c1 ; COc1cc(C(=O)O)cc2c1nc(CN1CCC(c3cccc(OCc4ccc(C(F)F)cc4F)n3)CC1)n2C]

### General Procedure for Preparation of Intermediate 2:

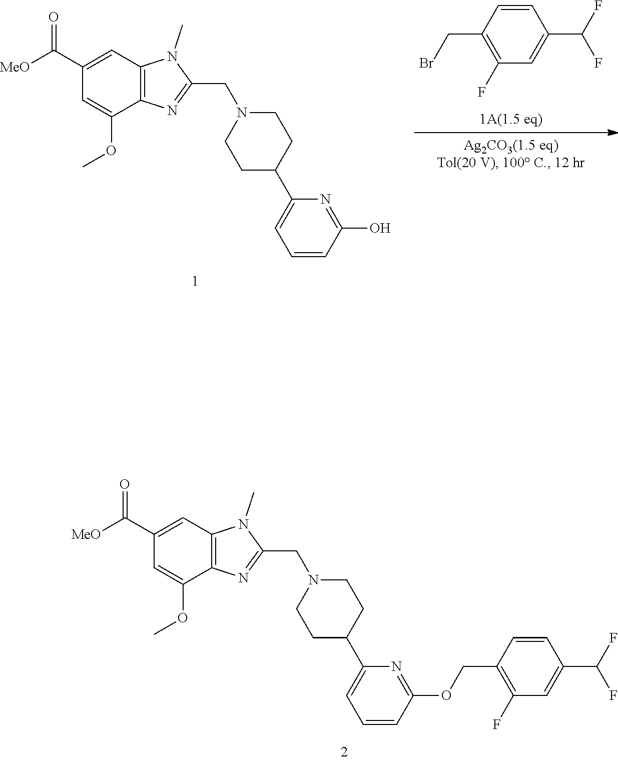 [SMILES: COC(=O)c1cc(OC)c2nc(CN3CCC(c4cccc(O)n4)CC3)n(C)c2c1 ; Fc1cc(C(F)F)ccc1CBr ; COC(=O)c1cc(OC)c2nc(CN3CCC(c4cccc(OCc5ccc(C(F)F)cc5F)n4)CC3)n(C)c2c1]

A mixture of 1 (90 mg, 219.26 μmol, 1 eq), 1A (119.12 mg, 328.90 μmol, 1.5 eq), Ag₂CO₃ (90.69 mg, 328.90 μmol, 14.92 μL, 1.5 eq) in Toluene (2 mL) was degassed and purged with N₂ for 3 times, and then the mixture was stirred at 100° C. for 12 hr under N₂ atmosphere. LC-MS (Rt=1.593 min) showed 1 was consumed completely and one main peak with desired mass was detected. The reaction mixture was diluted with H₂O 20 mL and extracted with EtOAc 60 mL (20 mL*3). The combined organic layers were washed with brine 30 mL, dried over Na₂SO₄, filtered, and concentrated under reduced pressure to give a residue. The residue was purified by column chromatography (SiO₂, Ethyl acetate: Methanol=100/1 to 10/1). 2 (60 mg, 105.53 μmol, 48.13% yield) was obtained as a yellow solid.

LCMS: RT=1.593 min, MS cal.: 568.23, [M+H]⁺=569.3

¹H NMR (400 MHz, MeOH-d4) δ=7.91-7.84 (m, 1H), 7.65-7.52 (m, 2H), 7.42 (s, 1H), 7.35-7.25 (m, 2H), 6.80 (d, J=7.2 Hz, 1H), 6.74 (t, J=56 Hz, 1H), 6.64 (d, J=8.2 Hz, 1H), 5.46 (s, 2H), 4.04-3.93 (m, 10H), 3.87 (s, 2H), 2.98 (br d, J=11.1 Hz, 2H), 2.62 (td, J=7.6, 15.0 Hz, 1H), 2.33-2.24 (m, 2H), 1.90-1.77 (m, 4H)

### General Procedure for Preparation of Compound 305 (3):

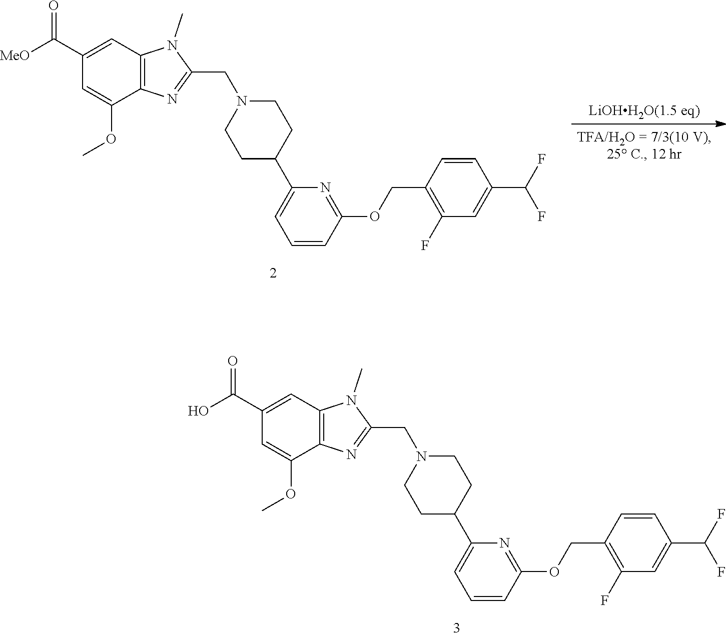 [SMILES: COC(=O)c1cc(OC)c2nc(CN3CCC(c4cccc(OCc5ccc(C(F)F)cc5F)n4)CC3)n(C)c2c1 ; COc1cc(C(=O)O)cc2c1nc(CN1CCC(c3cccc(OCc4ccc(C(F)F)cc4F)n3)CC1)n2C]

A mixture of 2 (50 mg, 87.94 μmol, 1 eq), LiOH·H₂O (5.54 mg, 131.91 μmol, 1.5 eq) in THF (0.7 mL) and H₂O (0.3 mL) was degassed and purged with N₂ for 3 times, and then the mixture was stirred at 25° C. for 12 hr under N₂ atmosphere. LC-MS (product, RT=1.102 min) showed 2 was consumed completely and one main peak with desired mass was detected. The reaction was purified directly. The residue was purified by prep-HPLC (column: Waters Xbridge Prep OBD C18 150*40 mm*10 um; mobile phase: [H₂O (10 mM NH₄HCO₃)-ACN]; gradient: 20%-70% B over 8.0 min). 2-((4-(6-((4-(Difluoromethyl)-2-fluorobenzyl)oxy)pyridin-2-yl)piperidin-1-yl)methyl)-4-methoxy-1-methyl-1H-benzo[d]imidazole-6-carboxylic acid (27.05 mg, 47.83 μmol, 54.39% yield, 98.06% purity) was obtained as a white solid.

LCMS: RT=2.279 min, MS cal.: 554.21, [M+1]⁺=555.3

HPLC: RT=10.76 min, purity: 98.06%

¹H NMR (400 MHz, MeOH-d4) δ=7.89-7.84 (m, 1H), 7.64-7.54 (m, 2H), 7.45 (s, 1H), 7.31 (br t, J=10.4 Hz, 2H), 6.82 (d, J=7.2 Hz, 1H), 6.75 (t, J=56 Hz, 1H), 6.65 (d, J=8.4 Hz, 1H), 6.61 (s, 1H), 5.47 (s, 2H), 4.03 (s, 3H), 3.98 (s, 3H), 3.94 (s, 2H), 3.06 (br d, J=11.2 Hz, 2H), 2.70-2.60 (m, 1H), 2.42-2.32 (m, 2H), 1.91-1.82 (m, 4H)

### Example A80—Preparation of Compound 342. 2-(4-(6-((4-cyano-2-fluorobenzyl)oxy)pyridin-2-yl)-2-fluorobenzyl)-4-methoxy-1-methyl-1H-benzo[d]imidazole-6-carboxylic acid (Compound 342) (4)

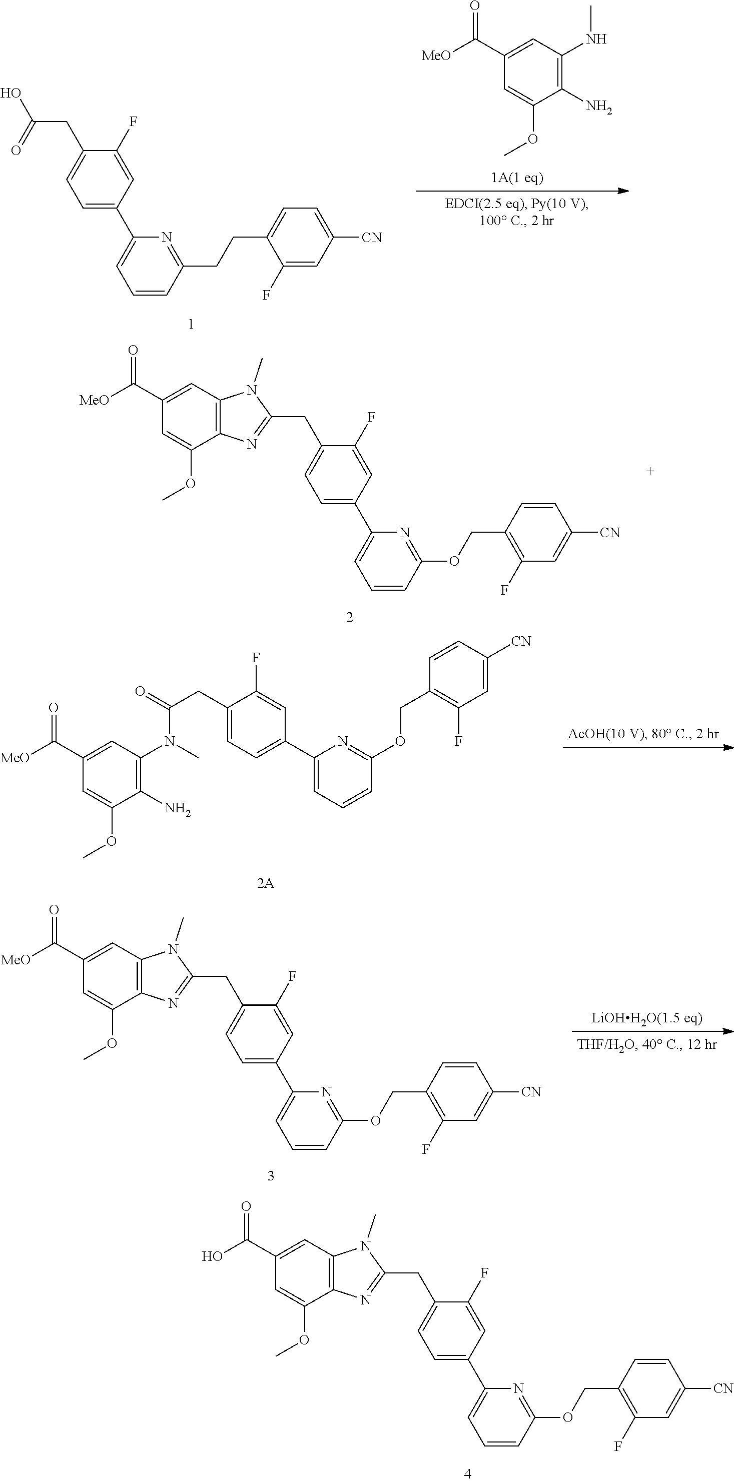 [SMILES: N#Cc1ccc(CCc2cccc(-c3ccc(CC(=O)O)c(F)c3)n2)c(F)c1 ; CNc1cc(C(=O)OC)cc(OC)c1N ; COC(=O)c1cc(OC)c2nc(Cc3ccc(-c4cccc(OCc5ccc(C#N)cc5F)n4)cc3F)n(C)c2c1 ; COC(=O)c1cc(OC)c(N)c(N(C)C(=O)Cc2ccc(-c3cccc(OCc4ccc(C#N)cc4F)n3)cc2F)c1 ; COC(=O)c1cc(OC)c2nc(Cc3ccc(-c4cccc(OCc5ccc(C#N)cc5F)n4)cc3F)n(C)c2c1 ; COc1cc(C(=O)O)cc2c1nc(Cc1ccc(-c3cccc(OCc4ccc(C#N)cc4F)n3)cc1F)n2C]

### General Procedure for Preparation of Intermediate 2 & 2A:

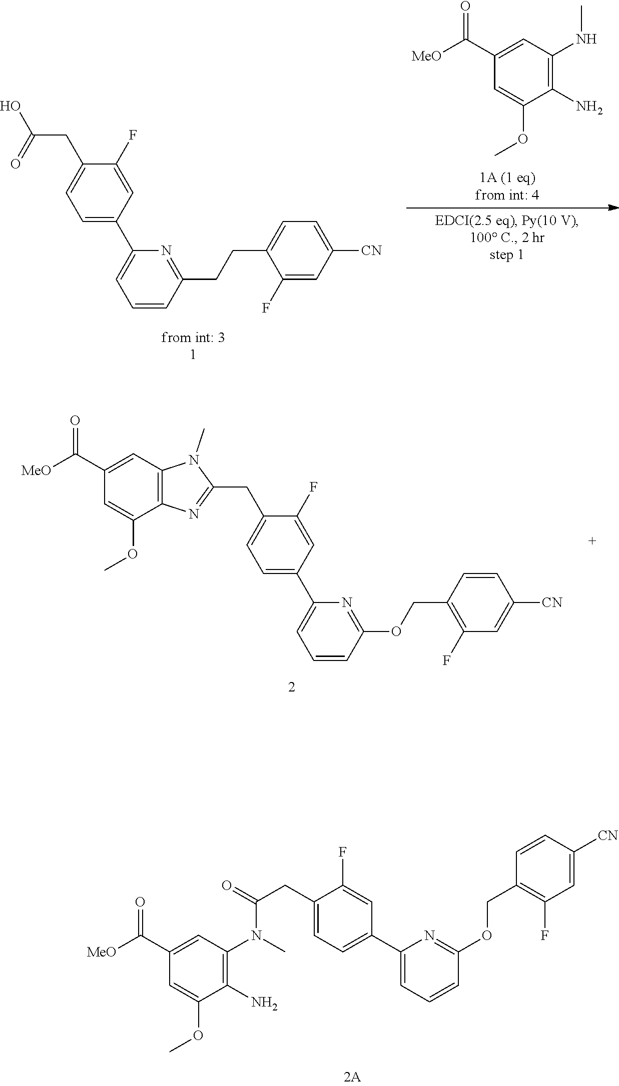 [SMILES: N#Cc1ccc(CCc2cccc(-c3ccc(CC(=O)O)c(F)c3)n2)c(F)c1 ; CNc1cc(C(=O)OC)cc(OC)c1N ; COC(=O)c1cc(OC)c2nc(Cc3ccc(-c4cccc(OCc5ccc(C#N)cc5F)n4)cc3F)n(C)c2c1 ; COC(=O)c1cc(OC)c(N)c(N(C)C(=O)Cc2ccc(-c3cccc(OCc4ccc(C#N)cc4F)n3)cc2F)c1]

To a solution of 1 (120 mg, 315.50 μmol, 1 eq) and 1A (66.33 mg, 315.50 μmol, 1 eq) in Py (1.2 mL) was added EDCI (151.21 mg, 788.76 μmol, 2.5 eq) at 20° C. The mixture was stirred at 100° C. for 2 hr. LC-MS showed 1 was consumed completely and desired mass was detected. The reaction mixture was adjusted to pH=3 with 1M FA. Then 10 mL H₂O was added to the mixture and extracted with EtOAc (10 mL*3). The combined organic phase was washed with brine (10 mL*2), dried with anhydrous Na₂SO₄, filtered and concentrated in vacuum. The crude product 2 & 2A (640 mg, crude, ratio: 1:2 in LCMS, a yellow solid) was used into the next step without further purification.

LCMS (Compound 2): RT=1.819 min, MS cal.: 554.2, [M+H]⁺=555.3

LCMS (Compound 2A): RT=2.131 min, MS cal.: 572.2, [M+H]⁺=573.2

### General Procedure for Preparation of Intermediate 3:

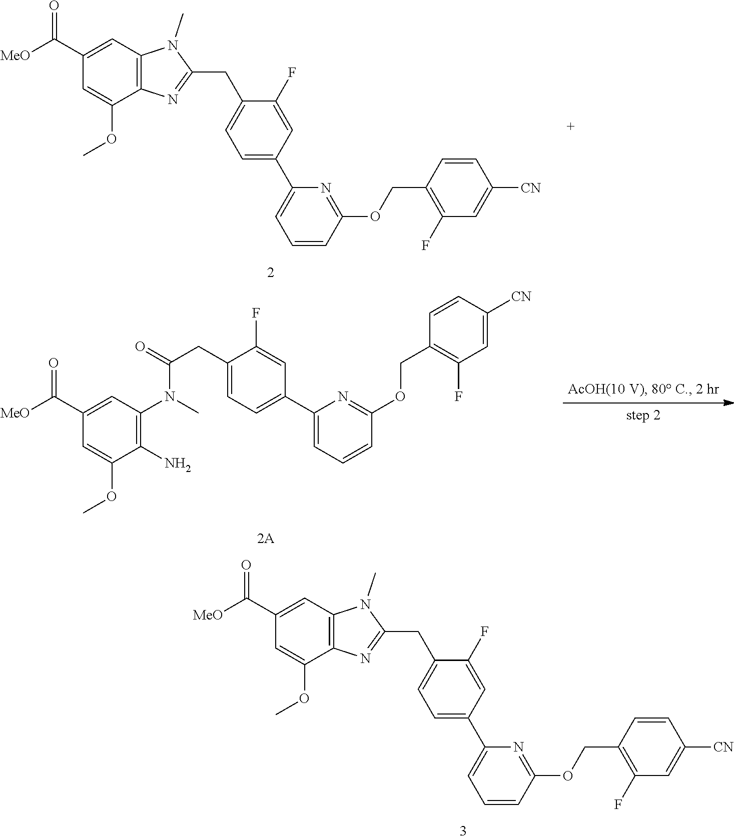 [SMILES: COC(=O)c1cc(OC)c2nc(Cc3ccc(-c4cccc(OCc5ccc(C#N)cc5F)n4)cc3F)n(C)c2c1 ; COC(=O)c1cc(OC)c(N)c(N(C)C(=O)Cc2ccc(-c3cccc(OCc4ccc(C#N)cc4F)n3)cc2F)c1 ; COC(=O)c1cc(OC)c2nc(Cc3ccc(-c4cccc(OCc5ccc(C#N)cc5F)n4)cc3F)n(C)c2c1]

2 & 2A (640 mg, crude, ratio: 1:2 in LCMS) was dissolved in CH₃COOH (6 mL) at 25° C. The mixture was stirred at 80° C. for 2 hr. LC-MS showed 2 & 2A was consumed completely and desired mass was detected. The reaction mixture was concentrated under reduced pressure to remove CH₃COOH. The reaction mixture was diluted with sat. NaHCO₃20 mL and extracted with EtOAc (10 mL*3). The combined organic layers were washed with brine 20 mL, dried over anhydrous Na₂SO₄, filtered and concentrated under reduced pressure to give a residue. The residue was purified by column chromatography (SiO₂, Petroleum ether/Ethyl acetate=5/1 to 1/0). 3 (158 mg, 284.92 μmol, 25.49% yield) was obtained as a white solid.

LCMS: RT=1.798 min, MS cal.: 554.2, [M+H]⁺=555.3

¹H NMR (400 MHz, CHCl3-d) δ=7.74 (s, 1H), 7.77-7.61 (m, 1H), 7.73-7.61 (m, 3H), 7.49-7.39 (m, 3H), 7.34 (d, J=7.2 Hz, 1H), 6.83-6.77 (m, 1H), 5.60 (s, 2H), 4.47 (br s, 2H), 4.10 (s, 3H), 3.98-3.95 (m, 3H), 3.74 (s, 3H)

### General Procedure for Preparation of Compound 342 (4) Compound 4:

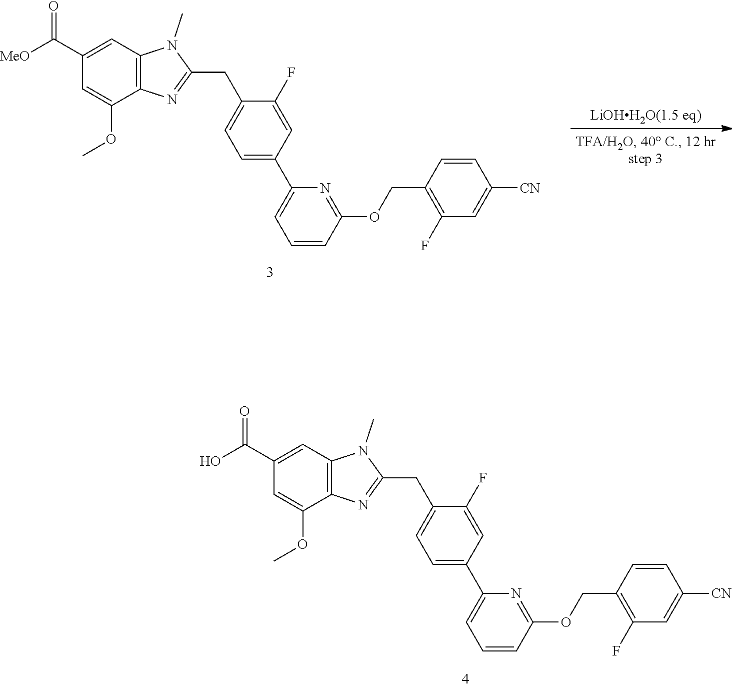 [SMILES: COC(=O)c1cc(OC)c2nc(Cc3ccc(-c4cccc(OCc5ccc(C#N)cc5F)n4)cc3F)n(C)c2c1 ; COc1cc(C(=O)O)cc2c1nc(Cc1ccc(-c3cccc(OCc4ccc(C#N)cc4F)n3)cc1F)n2C]

To a solution of 3 (128 mg, 230.82 μmol, 1 eq) in THF (8 mL) was added LiOH·H₂O (14.53 mg, 346.23 μmol, 1.5 eq) in H₂O (1.2 mL). The mixture was stirred at 40° C. for 12 hr. LC-MS (RT=1.121 min) showed ˜30% of 3 remained. Several new peaks were shown on LC-MS and ˜60% of desired Compound was detected. The residue was purified by prep-HPLC (column: Waters Xbridge Prep OBD C18 150*40 mm*10 um; mobile phase: [H₂O (10 mM NH₄HCO₃)-ACN]; gradient: 25%-65% B over 8.0 min). 2-(4-(6-((4-Cyano-2-fluorobenzyl)oxy)pyridin-2-yl)-2-fluorobenzyl)-4-methoxy-1-methyl-1H-benzo[d]imidazole-6-carboxylic acid (26.6 mg, 49.21 μmol, 21.32% yield, 98.31% purity) was obtained as a white solid.

LCMS: RT=2.454 min, MS cal.: 540.2, [M+H]⁺=541.2

HPLC: RT=11.759 min, purity: 98.31%

¹H NMR (400 MHz, DMSO-d₆) δ=7.92 (d, J=9.2 Hz, 1H), 7.89-7.83 (m, 3H), 7.79-7.71 (m, 1H), 7.74-7.71 (m, 1H), 7.65 (d, J=7.2 Hz, 1H), 7.38 (t, J=8.4 Hz, 1H), 7.26 (s, 1H), 6.92 (d, J=8.4 Hz, 1H), 5.61 (s, 2H), 4.37 (s, 2H), 3.90 (s, 3H), 3.82 (s, 3H)

### Example A81—Preparation of Compound 306. 4-Methoxy-2-((4-(6-((2-methoxy-4-(trifluoromethyl) benzyl)oxy)pyridin-2-yl)piperidin-1-yl)methyl)-1-methyl-1H-benzo[d]imidazole-6-carboxylic acid (Compound 306) (3)

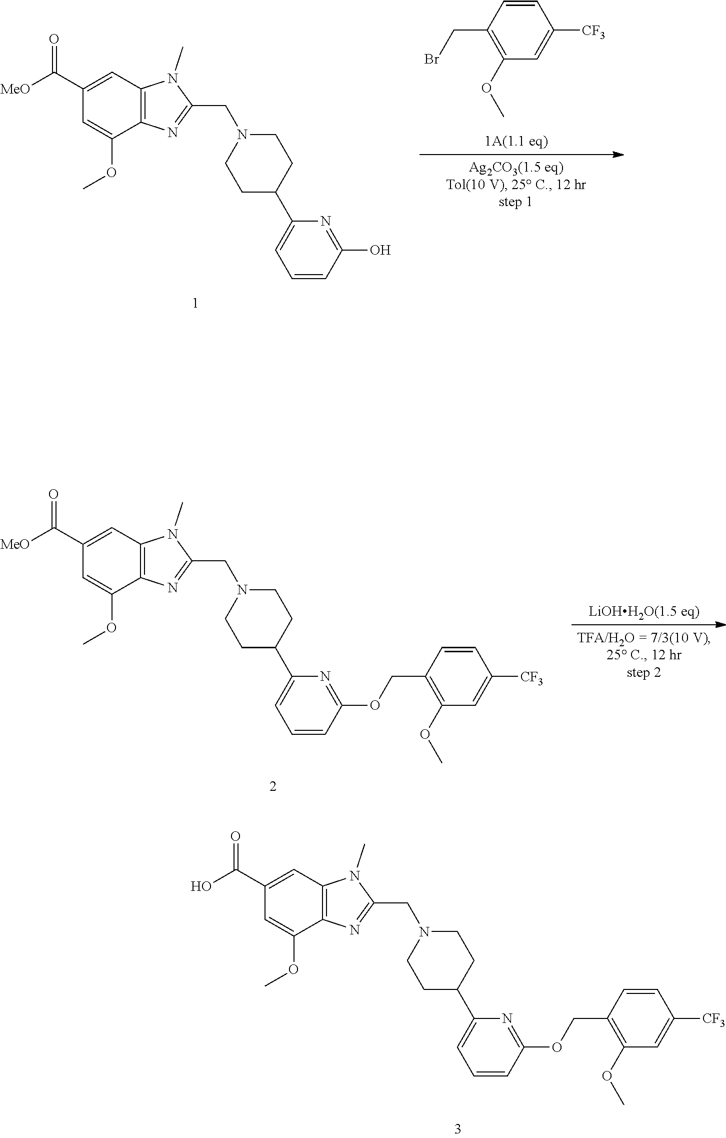 [SMILES: COC(=O)c1cc(OC)c2nc(CN3CCC(c4cccc(O)n4)CC3)n(C)c2c1 ; COc1cc(C(F)(F)F)ccc1CBr ; COC(=O)c1cc(OC)c2nc(CN3CCC(c4cccc(OCc5ccc(C(F)(F)F)cc5OC)n4)CC3)n(C)c2c1 ; COc1cc(C(F)(F)F)ccc1COc1cccc(C2CCN(Cc3nc4c(OC)cc(C(=O)O)cc4n3C)CC2)n1]

### General Procedure for Preparation of Intermediate 2:

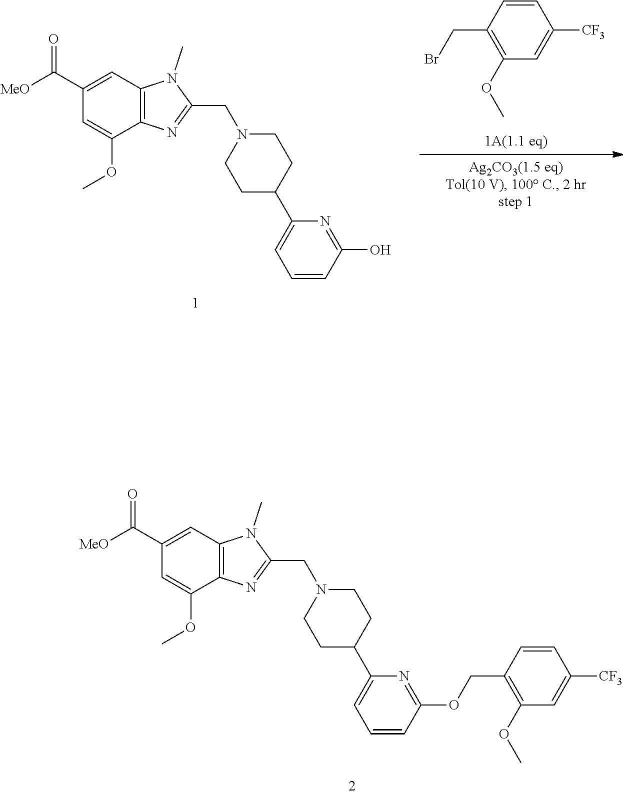 [SMILES: COC(=O)c1cc(OC)c2nc(CN3CCC(c4cccc(O)n4)CC3)n(C)c2c1 ; COc1cc(C(F)(F)F)ccc1CBr ; COC(=O)c1cc(OC)c2nc(CN3CCC(c4cccc(OCc5ccc(C(F)(F)F)cc5OC)n4)CC3)n(C)c2c1]

To a solution of 1 (120 mg, 292.35 μmol, 1 eq) in Toluene (1.5 mL) was added Ag₂CO₃ (120.92 mg, 438.53 μmol, 1.5 eq) and 1A (86.53 mg, 321.59 μmol, 1.1 eq). The mixture was stirred at 100° C. for 2 hr. LCMS showed 1 was consumed completely and one main peak with desired mass was detected. The reaction mixture was diluted with H₂O 10 mL and extracted with EtOAc 20 mL (10 mL*2). The combined organic layers were washed with brine 10 mL, dried over

Na₂SO₄, filtered, and concentrated under reduced pressure to give a residue. The residue was purified by column chromatography (SiO₂, PE/EtOAc=1/0 to 3/7). 2 (100 mg, 163.71 μmol, 56.00% yield) was obtained as a yellow solid.

LCMS: RT=1.672 min, MS cal.: 598.2/599.2, [M+1]⁺=599.3/600.3

¹HNMR (400 MHz, MeOH-d4) δ=7.88-7.83 (m, 1H), 7.61-7.51 (m, 2H), 7.41 (d, J=1.2 Hz, 1H), 7.24-7.17 (m, 2H), 6.83-6.78 (m, 1H), 6.68-6.63 (m, 1H), 5.42 (s, 2H), 4.02 (s, 3H), 3.97 (s, 3H), 3.95-3.93 (m, 3H), 3.93-3.91 (m, 3H), 3.85 (s, 2H), 2.98 (br d, J=11.6 Hz, 2H), 2.61 (tt, J=5.2, 10.3 Hz, 1H), 2.28 (dt, J=4.0, 11.1 Hz, 2H), 1.87-1.74 (m, 4H)

### General Procedure for Preparation of Compound 306 (3):

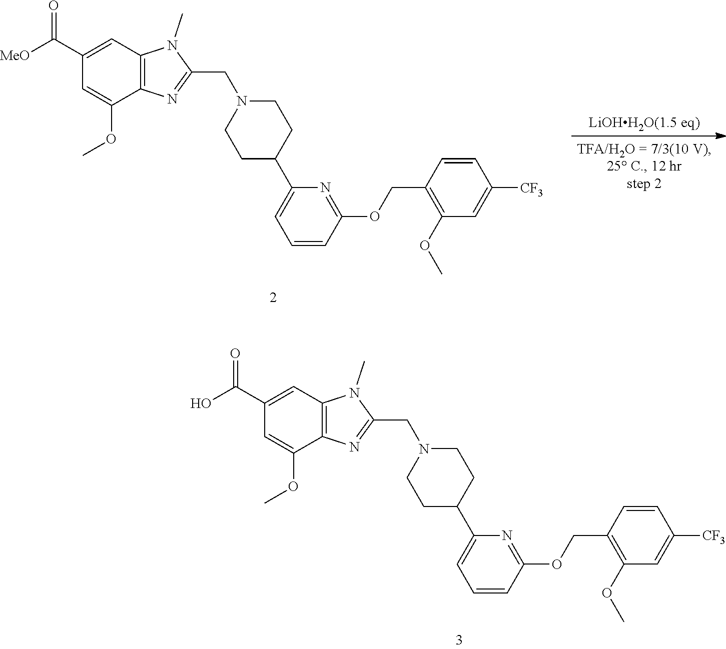 [SMILES: COC(=O)c1cc(OC)c2nc(CN3CCC(c4cccc(OCc5ccc(C(F)(F)F)cc5OC)n4)CC3)n(C)c2c1 ; COc1cc(C(F)(F)F)ccc1COc1cccc(C2CCN(Cc3nc4c(OC)cc(C(=O)O)cc4n3C)CC2)n1]

To a solution of 2 (95 mg, 158.70 μmol, 1 eq) in THF (1.4 mL) and H₂O (0.7 mL) was added LiOH·H₂O (9.99 mg, 238.05 μmol, 1.5 eq). The mixture was stirred at 25° C. for 12 hr. LCMS showed 26% of 2 remained. Several new peaks were shown on LCMS and 72% of desired product was detected. The mixture was added THF 0.1 mL. The residue was purified by prep-HPLC (neutral condition: column: Waters Xbridge Prep OBD C18 150*40 mm*10 um; mobile phase: [H₂O (10 mM NH₄HCO₃)-ACN]; gradient: 30%-60% B over 8.0 min). 4-Methoxy-2-((4-(6-((2-methoxy-4-(trifluoromethyl) benzyl)oxy)pyridin-2-yl)piperidin-1-yl)methyl)-1-methyl-1H-benzo[d]imidazole-6-carboxylic acid (28.95 mg, 49.03 μmol, 30.89% yield) was obtained as a white solid.

LCMS: RT=2.751 min, MS cal.: 584.2/585.2, [M+H]⁺=585.3/586.3

HPLC: RT=11.894 min

¹HNMR (400 MHz, MeOH-d4) δ=7.87-7.84 (m, 1H), 7.61-7.53 (m, 2H), 7.45 (d, J=0.8 Hz, 1H), 7.23-7.20 (m, 2H), 6.84-6.80 (m, 1H), 6.67 (d, J=8.0 Hz, 1H), 5.43 (s, 2H), 4.03 (s, 3H), 3.97 (s, 3H), 3.93 (s, 3H), 3.91 (s, 2H), 3.04 (br d, J=11.2 Hz, 2H), 2.64 (td, J=4.8, 9.6 Hz, 1H), 2.39-2.30 (m, 2H), 1.90-1.80 (m, 4H)

### Example A82—Preparation of Compound 343. 2-((4-(6-((4-Chloro-2-fluorobenzyl)oxy)pyridin-2-yl)piperidin-1-yl)methyl)-4-(2-hydroxyethoxy)-1-methyl-1H-benzo[d]imidazole-6-carboxylic acid (Compound 343) (3)

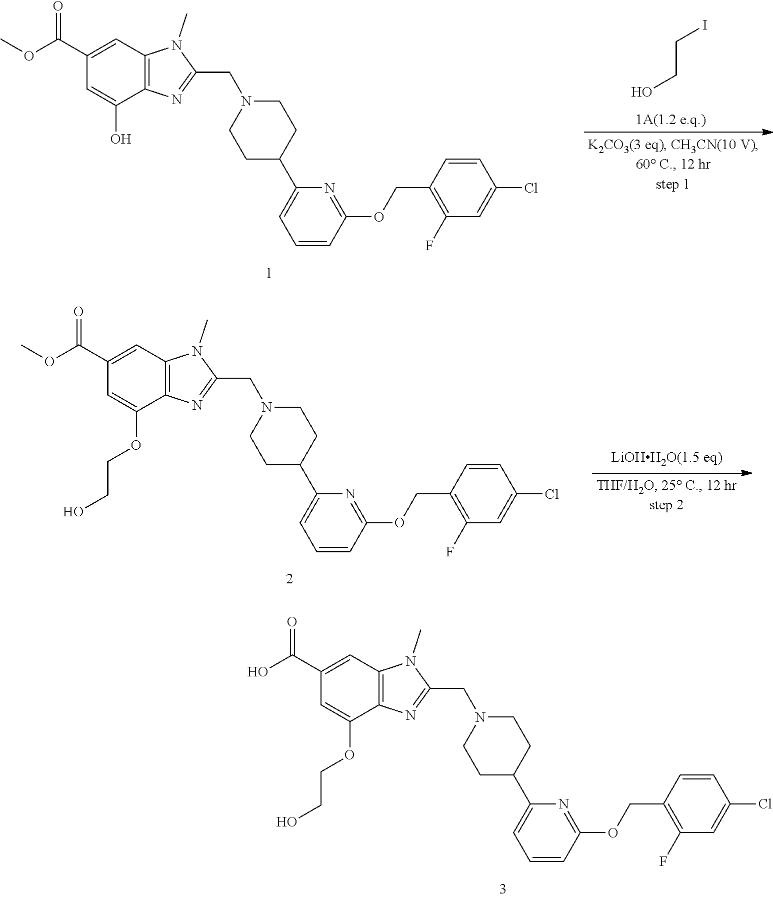

### General Procedure for Preparation of Intermediate 2:

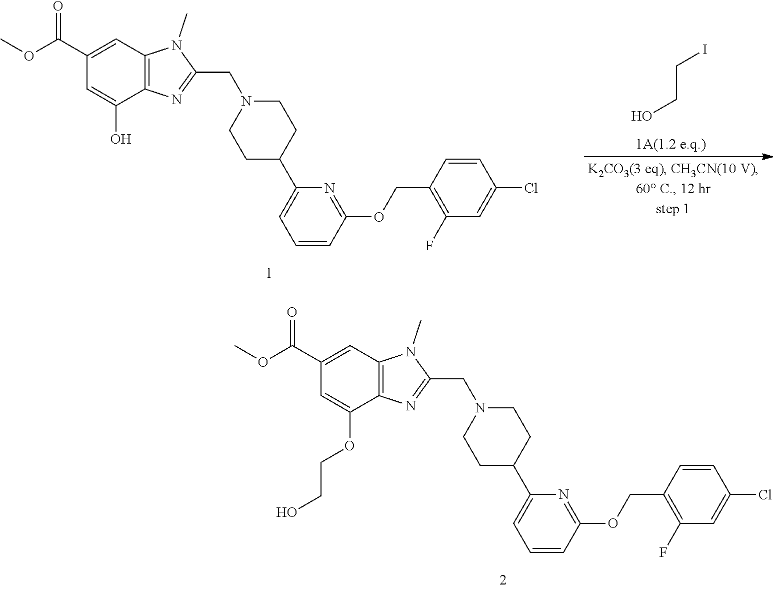

A mixture of 1 (200 mg, 371.06 μmol, 1 eq), 1A (95.71 mg, 556.59 μmol, 43.51 μL, 1.5 eq), K₂CO₃ (153.85 mg, 1.11 mmol, 3 eq) in ACN (2 mL) was degassed and purged with N₂ for 3 times, and then the mixture was stirred at 60° C. for 12 hr under N₂ atmosphere. LCMS showed ˜36% of 1 remained. Several new peaks were shown on LCMS and ˜29% of desired Compound was detected. The reaction mixture was diluted with H₂O (10 mL) and extracted with EtOAc (10 mL*3). The combined organic layers were washed with brine (10 mL*3), dried over Na₂SO₄, filtered and concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 55%-85% B over 8.0 min). 2 (37 mg, 63.46 μmol, 17.10% yield) was obtained as a white solid.

LCMS: RT=9.760 min, MS cal.: 582.2, [M+H]⁺=583.3

¹H NMR (400 MHz, MeOH-d4) δ=7.89 (d, J=1.2 Hz, 1H), 7.59-7.53 (m, 1H), 7.51-7.45 (m, 1H), 7.42 (d, J=1.2 Hz, 1H), 7.18 (s, 2H), 6.81 (d, J=7.2 Hz, 1H), 6.62 (d, J=7.6 Hz, 1H), 5.39 (s, 2H), 4.26 (s, 2H), 4.01-3.96 (m, 5H), 3.94 (s, 3H), 3.89 (s, 2H), 3.06-2.98 (m, 2H), 2.67-2.59 (m, 1H), 2.36-2.24 (m, 2H), 1.85 (br d, J=3.2 Hz, 4H)

### General Procedure for Preparation of Compound 343 (3):

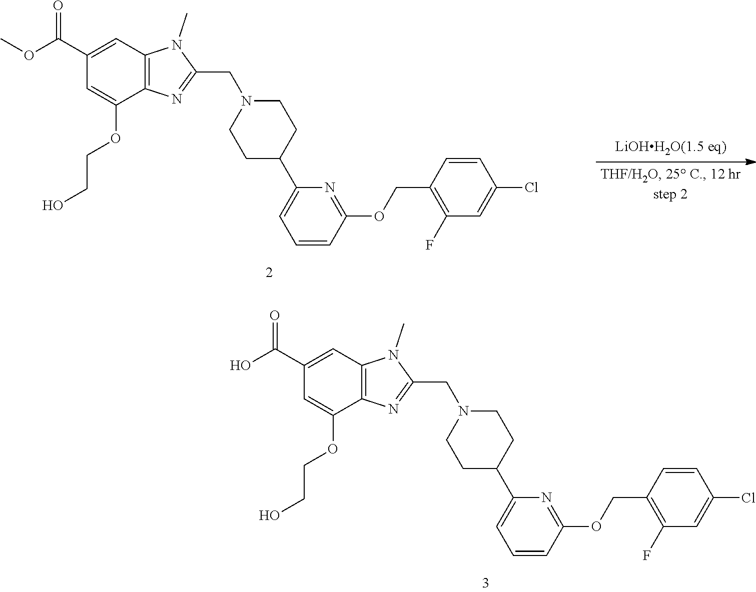 [SMILES: COC(=O)c1cc(OCCO)c2nc(CN3CCC(c4cccc(OCc5ccc(Cl)cc5F)n4)CC3)n(C)c2c1 ; Cn1c(CN2CCC(c3cccc(OCc4ccc(Cl)cc4F)n3)CC2)nc2c(OCCO)cc(C(=O)O)cc21]

A mixture of 2 (27 mg, 46.31 μmol, 1 eq), LiOH·H₂O (2.91 mg, 69.46 μmol, 1.5 eq) in THF (0.7 mL) and H₂O (0.3 mL) was degassed and purged with N₂ for 3 times, and then the mixture was stirred at 25° C. for 12 hr under N₂ atmosphere. LCMS showed 2 was consumed completely and one main peak with desired mass was detected. The reaction mixture was added with H₂O (10 mL) and extracted with EtOAc (10 mL*3). The combined organic layers were washed with brine (10 mL*3), dried over Na₂SO₄, filtered and concentrated under reduced pressure to give a residue. The residue was purified by prep-HPLC (column: Waters Xbridge BEH C18 100* 30 mm* 10 um; mobile phase: [H₂O (10 mM NH₄HCO₃)-ACN]; gradient: 25%-55% B over 8.0 min). 2-((4-(6-((4-Chloro-2-fluorobenzyl)oxy)pyridin-2-yl)piperidin-1-yl)methyl)-4-(2-hydroxyethoxy)-1-methyl-1H-benzo[d]imidazole-6-carboxylic acid (8.23 mg, 14.46 μmol, 31.23% yield) was obtained as a white solid.

LCMS: RT=2.630 min, MS cal.: 568.2, [M+H]⁺=569.2

HPLC: RT=10.457 min, purity: 99.14%

1H NMR (400 MHz, DMSO-d6) δ=7.76 (s, 1H), 7.66-7.60 (m, 1H), 7.56 (t, J=8.4 Hz, 1H), 7.45 (dd, J=2.0, 10.0 Hz, 1H), 7.31-7.26 (m, 2H), 6.87 (d, J=7.2 Hz, 1H), 6.67 (d, J=8.4 Hz, 1H), 5.36 (s, 2H), 4.98-4.91 (m, 1H), 4.22 (t, J=5.2 Hz, 2H), 3.90 (s, 3H), 3.85-3.76 (m, 4H), 2.93 (br d, J=11.2 Hz, 2H), 2.61-2.56 (m, 1H), 2.19 (br s, 2H), 1.83-1.65 (m, 4H)

### Example A83—Preparation of Compound 344. 2-((4-(6-((4-Cyano-2-methoxybenzyl)oxy)pyridin-2-yl)piperidin-1-yl)methyl)-4-methoxy-1-methyl-1H-benzo[d]imidazole-6-carboxylic acid (Compound 344) (3)

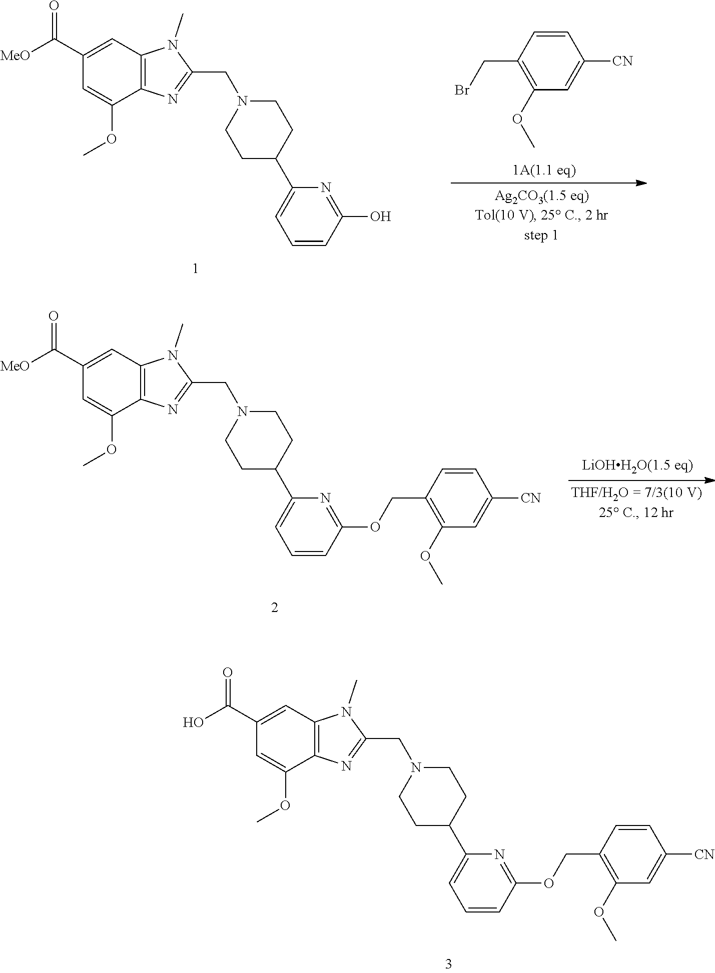 [SMILES: COC(=O)c1cc(OC)c2nc(CN3CCC(c4cccc(O)n4)CC3)n(C)c2c1 ; COc1cc(C#N)ccc1CBr ; COC(=O)c1cc(OC)c2nc(CN3CCC(c4cccc(OCc5ccc(C#N)cc5OC)n4)CC3)n(C)c2c1 ; COc1cc(C#N)ccc1COc1cccc(C2CCN(Cc3nc4c(OC)cc(C(=O)O)cc4n3C)CC2)n1]

### General Procedure for Preparation of Intermediate 2:

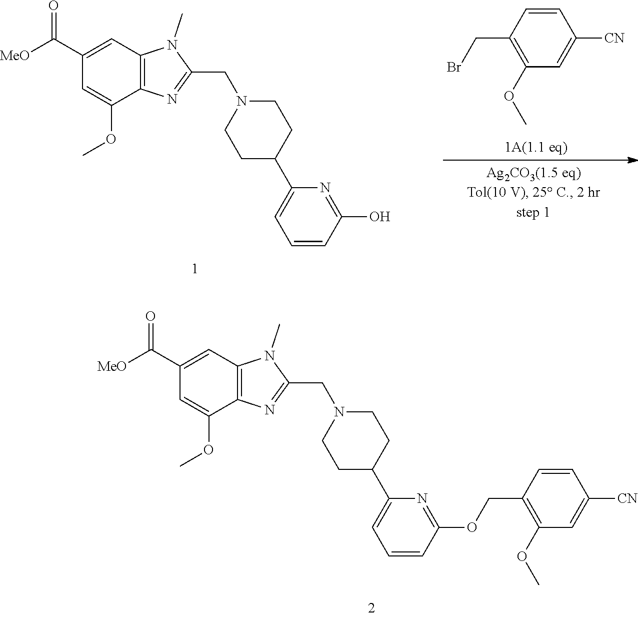 [SMILES: COC(=O)c1cc(OC)c2nc(CN3CCC(c4cccc(O)n4)CC3)n(C)c2c1 ; COc1cc(C#N)ccc1CBr ; COC(=O)c1cc(OC)c2nc(CN3CCC(c4cccc(OCc5ccc(C#N)cc5OC)n4)CC3)n(C)c2c1]

To a solution of 1 (120 mg, 292.35 μmol, 1 eq) in Toluene (1.5 mL) was added Ag₂CO₃ (120.92 mg, 438.53 μmol, 19.90 μL, 1.5 eq) and 1A (72.70 mg, 321.59 μmol, 1.1 eq). The mixture was stirred at 100° C. for 2 hr. LCMS showed 1 was consumed completely and one main peak with desired mass was detected. The reaction mixture was diluted with H₂O (10 mL) and extracted with EtOAc 20 mL (10 mL*2). The combined organic layers were washed with brine 10 mL, dried over Na₂SO₄, filtered and concentrated under reduced pressure to give a residue. The residue was purified by column chromatography (SiO₂, Petroleum ether/Ethyl acetate=9/1 to 0/1). 2 (95 mg, 170.98 μmol, 59.38% yield) was obtained as a yellow solid.

LCMS: RT=1.501 min, MS cal.: 555.2/556.3, [M+1]⁺=556.2/557.2

¹H NMR (400 MHz, CHCl3-d) δ=7.78 (s, 1H), 7.57-7.48 (m, 2H), 7.39 (s, 1H), 7.25 (s, 1H), 7.11 (s, 1H), 6.74 (d, J=7.2 Hz, 1H), 6.64 (d, J=8.0 Hz, 1H), 5.44 (s, 2H), 4.07 (s, 3H), 3.97 (s, 6H), 3.91 (s, 3H), 3.88 (br s, 2H), 3.06-2.90 (m, 2H), 2.69-2.54 (m, 1H), 2.28 (br s, 2H), 1.87 (br s, 2H), 1.83-1.69 (m, 2H)

### General Procedure for Preparation of Compound 344 (3) Compound 3:

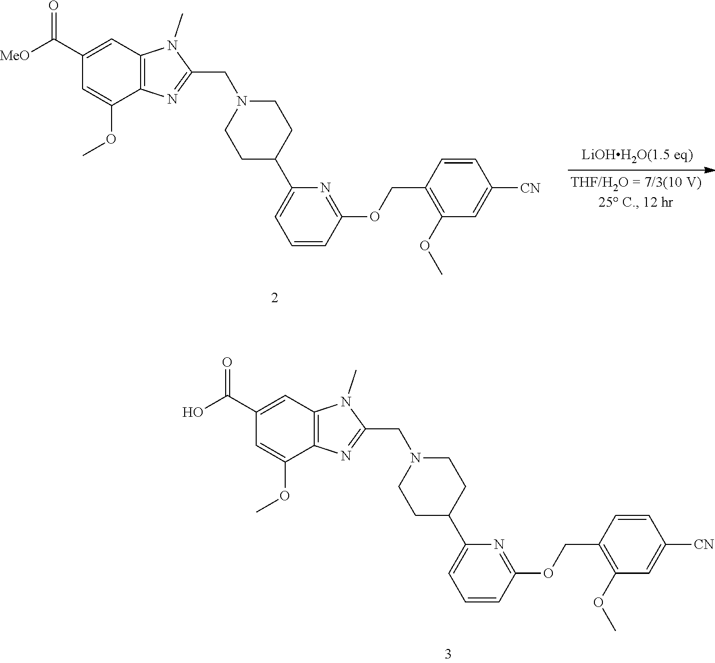 [SMILES: COC(=O)c1cc(OC)c2nc(CN3CCC(c4cccc(OCc5ccc(C#N)cc5OC)n4)CC3)n(C)c2c1 ; COc1cc(C#N)ccc1COc1cccc(C2CCN(Cc3nc4c(OC)cc(C(=O)O)cc4n3C)CC2)n1]

To a solution of 2 (70 mg, 125.98 μmol, 1 eq) in H₂O (0.3 mL) and THF (0.7 mL) was added LiOH·H₂O (7.93 mg, 188.98 μmol, 1.5 eq). The mixture was stirred at 25° C. for 12 hr. LCMS (product: RT=1.080 min; start material: RT=1.498 min) showed ˜23% of the 2 was remained. The crude product was purified by reversed-phase HPLC (column: Waters Xbridge BEH C18 100*30 mm*10 um; mobile phase: [H₂O (10 mM NH₄HCO₃)-ACN]; gradient: 25%-55% B over 8.0 min). 2-((4-(6-((4-Cyano-2-methoxybenzyl)oxy)pyridin-2-yl)piperidin-1-yl)methyl)-4-methoxy-1-methyl-1H-benzo[d]imidazole-6-carboxylic acid (26.57 mg, 49.06 μmol, 38.94% yield) was obtained as a white solid.

LCMS: RT=2.506 min, MS cal.: 541.6, [M+H]⁺=542.3

HPLC: RT=10.018 min, purity=99.75%

¹H NMR (400 MHz, DMSO-d₆) δ=7.72 (d, J=0.8 Hz, 1H), 7.63 (t, J=7.6 Hz, 1H), 7.54-7.50 (m, 2H), 7.41 (dd, J=1.2, 7.6 Hz, 1H), 7.28 (d, J=0.8 Hz, 1H), 6.86 (d, J=7.2 Hz, 1H), 6.71 (d, J=8.0 Hz, 1H), 5.36 (s, 2H), 3.93 (s, 3H), 3.89 (s, 3H), 3.86 (s, 3H), 3.77 (s, 2H), 2.91 (br d, J=11.2 Hz, 2H), 2.61-2.53 (m, 1H), 2.16 (br t, J=10.8 Hz, 2H), 1.79-1.71 (m, 2H), 1.71-1.59 (m, 2H)

### Example A84—Preparation of Compound 345. 2-((4-(6-((4-Chloro-2-fluorobenzyl)oxy)pyridin-2-yl)piperidin-1-yl)methyl)-4-hydroxy-1-methyl-1H-benzo[d]imidazole-5-carboxylic acid (Compound 345) (7)

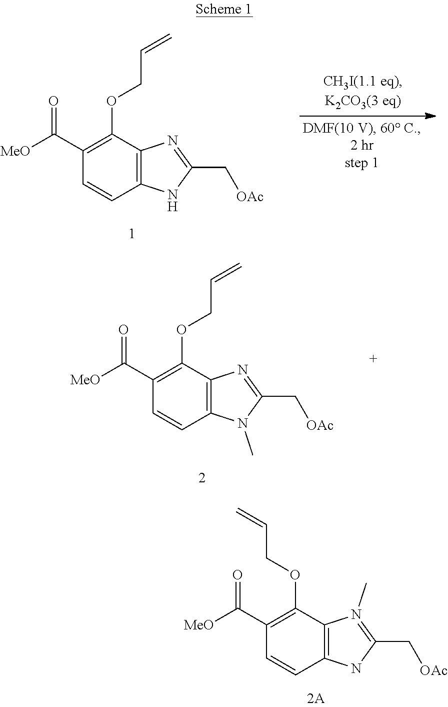

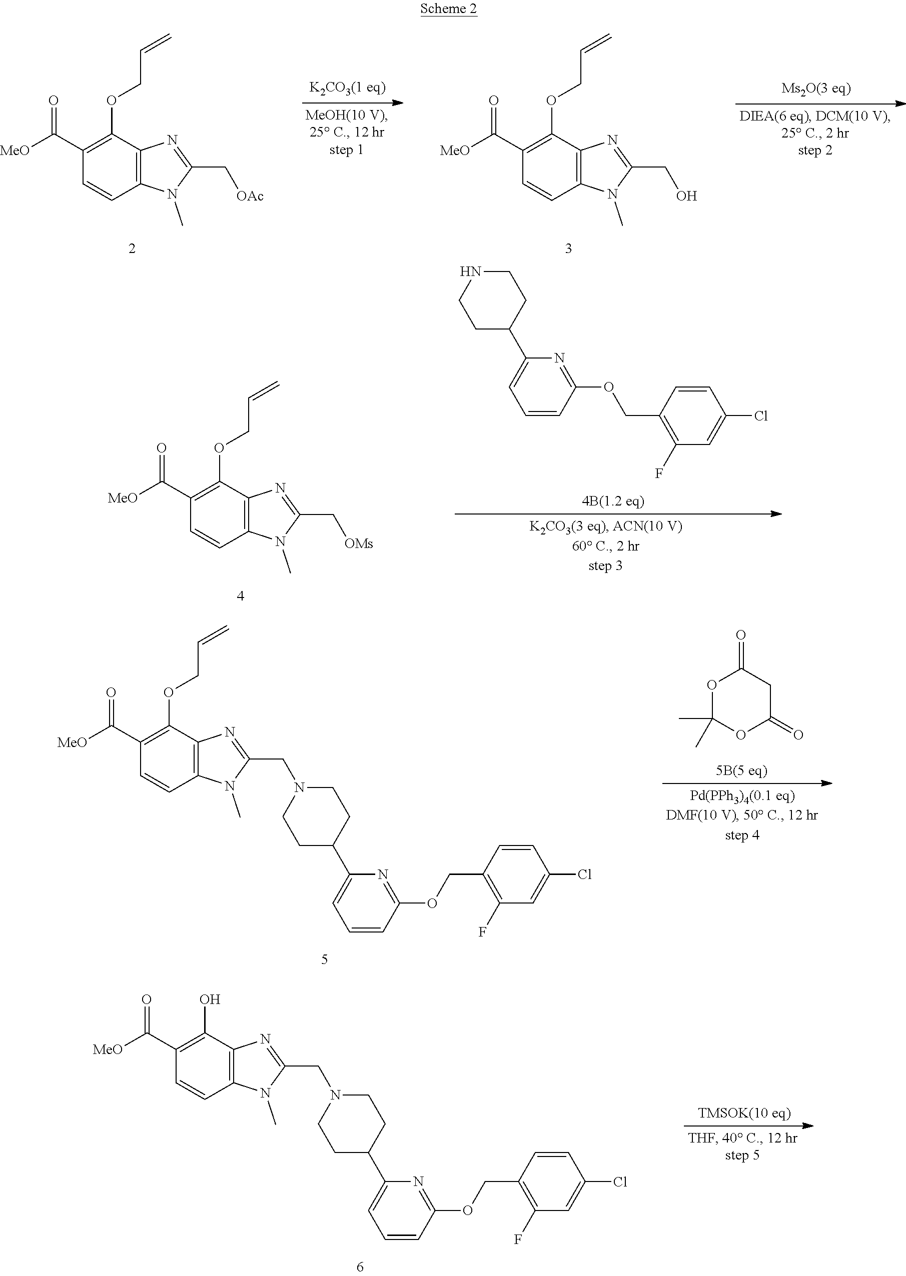

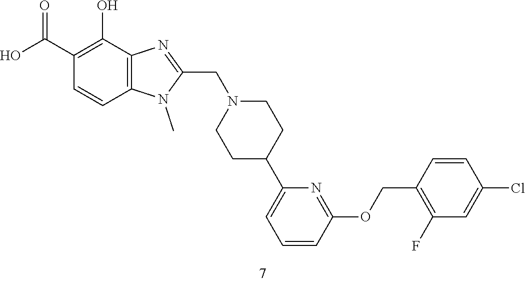 [SMILES: Cn1c(CN2CCC(c3cccc(OCc4ccc(Cl)cc4F)n3)CC2)nc2c(O)c(C(=O)O)ccc21]

### General Procedure for Preparation of Scheme 1, Intermediate 2:

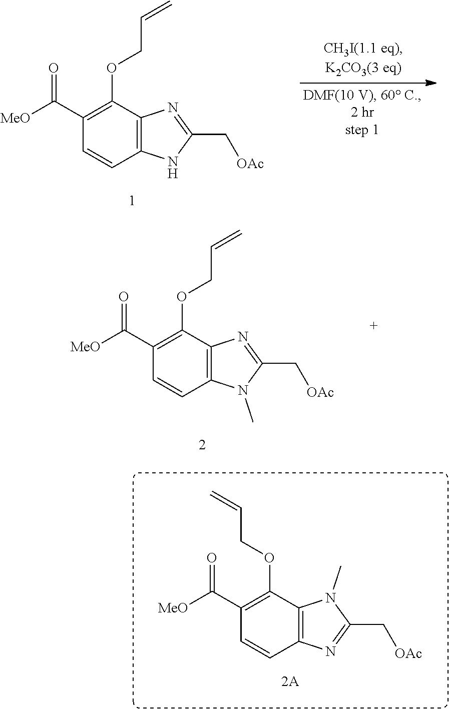 [SMILES: C=CCOc1c(C(=O)OC)ccc2[nH]c(COC(C)=O)nc12 ; C=CCOc1c(C(=O)OC)ccc2c1nc(COC(C)=O)n2C ; C=CCOc1c(C(=O)OC)ccc2nc(COC(C)=O)n(C)c12]

Equip a 50 mL three-necked round bottom flask, thermometer, N₂ balloon. DMF (15 mL) was charged to the three-necked round bottom flask, then 1 (1.5 g, 4.93 mmol, 1 eq) and K₂CO₃ (1.02 g, 7.39 mmol, 1.5 eq) was added to the mixture at 25° C. At 25° C. inner temperature, CH₃I (769.65 mg, 5.42 mmol, 337.56 μL, 1.1 eq) was added to the reaction mixture at 25° C. After the addition, the mixture was stirred at 25° C. for 2 hr. LCMS (product: RT=1.071 min) showed the starting material was consumed completely. After 2 hr, the reaction mixture was added to H₂O (10 mL) at 0° C. The mixture was extracted by EtOAc (20 mL). Then organic phase was combined and washed by brine (15 mL), dried with Na₂SO₄, filtered. The organic phase was concentrated under reduced pressure at 40° C. to give a residue. The residue was purified by column chromatography (SiO₂, Petroleum ether/Ethyl acetate=100/1 to 1/1). 2 (1 g, 3.14 mmol, 63.73% yield) was obtained as a white oil. 2A (270 mg, 848.19 μmol, 17.21% yield) was obtained as a white solid.

LCMS: RT2=1.070 min, MS cal.: 318.33, [M+H]⁺=318.9

¹H NMR (400 MHz, CHCl3-d) δ=7.81 (d, J=8.8 Hz, 1H), 7.00 (d, J=8.8 Hz, 1H), 6.19 (tdd, J=5.2, 10.6, 17.2 Hz, 1H), 5.48 (qd, J=1.6, 17.2 Hz, 1H), 5.37 (s, 2H), 5.35-5.30 (m, 2H), 5.23 (qd, J=1.2, 10.5 Hz, 1H), 3.92 (s, 3H), 3.81 (s, 3H)

LCMS: RT_{2A}=1.059 min, MS cal.: 318.33, [M+H]⁺=318.9

¹H NMR (400 MHz, CHCl3-d) δ=7.80 (d, J=8.4 Hz, 1H), 7.53 (d, J=8.8 Hz, 1H), 6.25-6.08 (m, 1H), 5.45 (dd, J=1.2, 17.2 Hz, 1H), 5.37 (s, 2H), 5.33 (dd, J=1.0, 10.4 Hz, 1H), 4.64 (d, J=5.6 Hz, 2H), 4.08 (s, 3H), 3.95 (s, 3H)

### General Procedure for Preparation of Scheme 2, Intermediate 3:

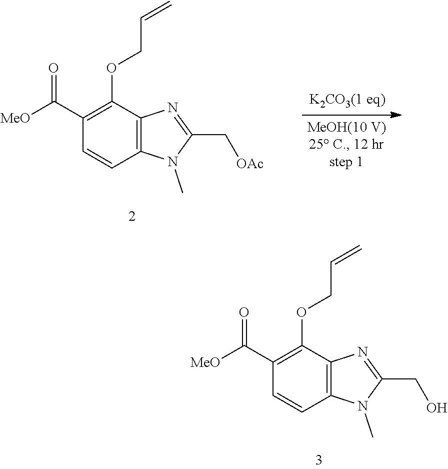 [SMILES: C=CCOc1c(C(=O)OC)ccc2c1nc(COC(C)=O)n2C ; C=CCOc1c(C(=O)OC)ccc2c1nc(CO)n2C]

To a solution of 2 (400 mg, 1.26 mmol, 1 eq) in MeOH (4 mL) was added K₂CO₃ (173.67 mg, 1.26 mmol, 1 eq). The mixture was stirred at 25° C. for 12 hr. LCMS (product: RT=0.908 min) showed the starting material was consumed completely. The reaction mixture was filtered, and the filtrate concentrated under reduced pressure to give a residue. The crude product 3 (340 mg, crude) was used into the next step without further purification.

LCMS: RT=0.920 min, MS cal.: 276.29, [M+H]⁺=276.9

1H NMR (400 MHz, DMSO-d6) δ=7.60-7.51 (m, 1H), 7.19 (d, J=8.4 Hz, 1H), 6.17-6.01 (m, 1H), 5.41 (br dd, J=1.2, 17.2 Hz, 1H), 5.22 (br d, J=5.2 Hz, 2H), 5.16 (br d, J=10.4 Hz, 1H), 4.71 (s, 2H), 3.83 (s, 3H), 3.81-3.76 (m, 3H), 3.16 (d, J=5.6 Hz, 1H)

### General Procedure for Preparation of Scheme 2, Intermediate 4:

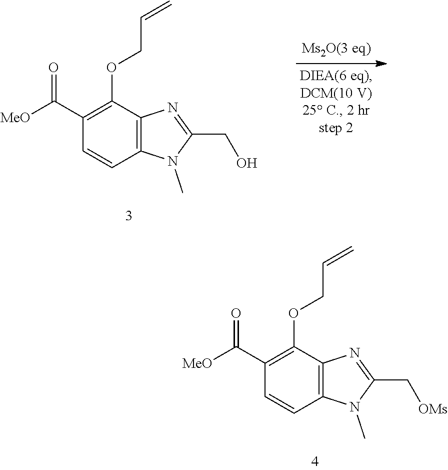 [SMILES: C=CCOc1c(C(=O)OC)ccc2c1nc(CO)n2C ; C=CCOc1c(C(=O)OC)ccc2c1nc(COS(C)(=O)=O)n2C]

A mixture of 3 (340 mg, 1.23 mmol, 1 eq), DIEA (954.28 mg, 7.38 mmol, 1.29 mL, 6 eq), Ms₂O (643.10 mg, 3.69 mmol, 3 eq) in DCM (3.4 mL) was degassed and purged with N₂ for 3 times, and then the mixture was stirred at 25° C. for 2 hr under N₂ atmosphere. LCMS (product: RT=1.059 min) showed the starting material was consumed completely. The reaction mixture was added FA (1 M) to pH=5 at 25° C. The mixture was extracted by DCM (10 mL*3). Then organic phase was combined and washed by H₂O (10 mL), brine (10 mL), dried with Na₂SO₄, filtered. The organic phase was concentrated under reduced pressure at 45° C. to give a residue. 4 (430 mg, crude) was obtained as a white solid.

LCMS: RT=1.059 min, MS cal.: 354.38, [M+H]⁺=354.9

### General Procedure for Preparation of Scheme 2, Intermediate 5:

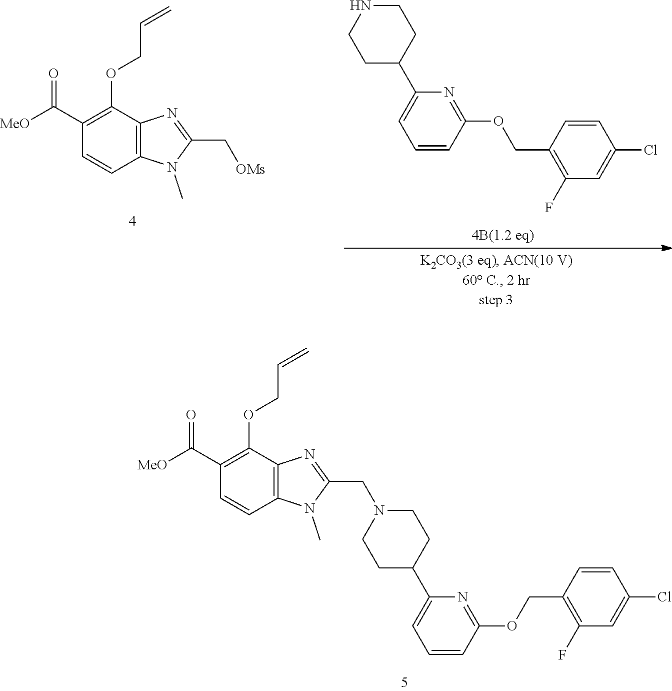 [SMILES: C=CCOc1c(C(=O)OC)ccc2c1nc(COS(C)(=O)=O)n2C ; Fc1cc(Cl)ccc1COc1cccc(C2CCNCC2)n1 ; C=CCOc1c(C(=O)OC)ccc2c1nc(CN1CCC(c3cccc(OCc4ccc(Cl)cc4F)n3)CC1)n2C]

A mixture of 4 (430 mg, 1.21 mmol, 1 eq), 4B (467.09 mg, 1.46 mmol, 1.2 eq), K₂CO₃ (503.09 mg, 3.64 mmol, 3 eq) in ACN (5 mL) was degassed and purged with N₂ for 3 times, and then the mixture was stirred at 60° C. for 2 hr under N₂ atmosphere. LCMS (product: RT=1.736 min) showed the starting material was consumed completely. The reaction mixture was diluted with H₂O (15 mL) and extracted with EtOAc (20 mL*3). The combined organic layers were washed with brine (20 mL*2), dried over Na₂SO₄, filtered and concentrated under reduced pressure to give a residue. The residue was purified by column chromatography (SiO₂, Petroleum ether/Ethyl acetate=100/1 to 0/1). 5 (410 mg, 708.04 μmol, 58.35% yield) was obtained as a white oil.

LCMS: RT=1.782 min, MS cal.: 579.07, [M+H]⁺=579.1

¹H NMR (400 MHz, CHCl3-d) δ=7.85-7.73 (m, 1H), 7.59-7.41 (m, 2H), 7.18-7.07 (m, 2H), 7.05-6.99 (m, 1H), 6.74 (br d, J=6.8 Hz, 1H), 6.66-6.57 (m, 1H), 6.26-6.11 (m, 1H), 5.52-5.44 (m, 1H), 5.43-5.38 (m, 2H), 5.36-5.28 (m, 2H), 5.21 (br d, J=9.6 Hz, 1H), 3.97-3.89 (m, 6H), 3.88-3.80 (m, 2H), 3.10-2.88 (m, 2H), 2.72-2.54 (m, 1H), 2.43-2.21 (m, 2H), 2.04-1.71 (m, 4H)

### General Procedure for Preparation of Scheme 2, Intermediate 6:

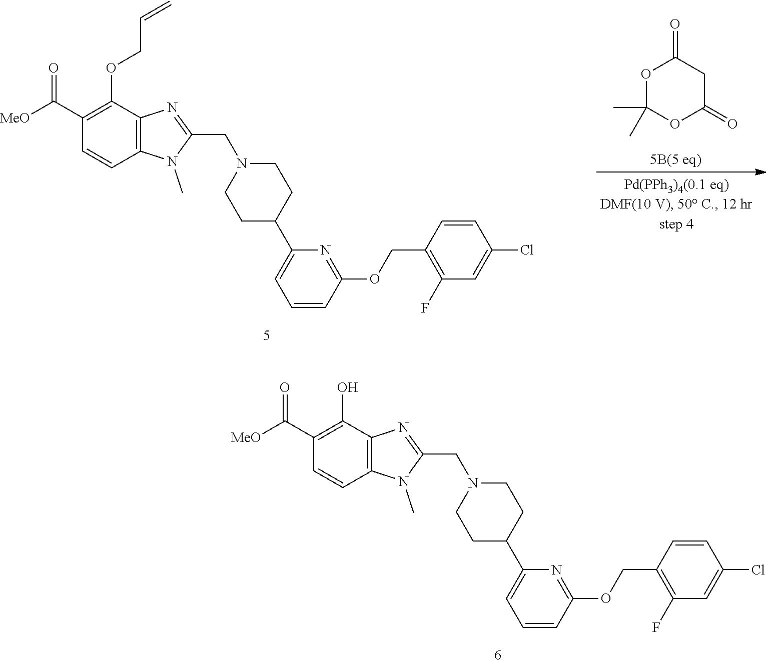

A mixture of 5 (300 mg, 518.08 μmol, 1 eq), 5B (373.34 mg, 2.59 mmol, 5 eq), Pd(PPh₃)₄ (59.87 mg, 51.81 μmol, 0.1 eq) in DMF (3 mL) was degassed and purged with N₂ for 3 times, and then the mixture was stirred at 50° C. for 12 hr under N₂ atmosphere. LCMS (product: RT=1.681 min) showed the starting material was consumed completely. The crude product was purified by reversed-phase HPLC (column: Waters Xbridge Prep OBD C18 150*40 mm*10 um; mobile phase: [H₂O (10 mM NH₄HCO₃)-ACN]; gradient: 50%-90% B over 8.0 min). 6 (160 mg, 296.85 μmol, 57.30% yield) was obtained as a yellow solid.

LCMS: RT=1.683 min, MS cal.: 539.00, [M+H]⁺=539.1

¹H NMR (400 MHz, CHCl3-d) δ=11.62-11.38 (m, 1H), 7.76 (d, J=8.8 Hz, 1H), 7.57-7.40 (m, 2H), 7.17-7.06 (m, 2H), 6.86 (d, J=8.8 Hz, 1H), 6.73 (d, J=7.2 Hz, 1H), 6.61 (d, J=8.0 Hz, 1H), 5.41 (s, 2H), 3.98 (s, 3H), 3.94 (br s, 3H), 3.90 (br d, J=2.8 Hz, 2H), 2.99 (br s, 2H), 2.72-2.54 (m, 1H), 2.29 (br dd, J=5.6, 7.2 Hz, 2H), 1.96-1.74 (m, 4H)

### General Procedure for Preparation of Compound 345 (7):

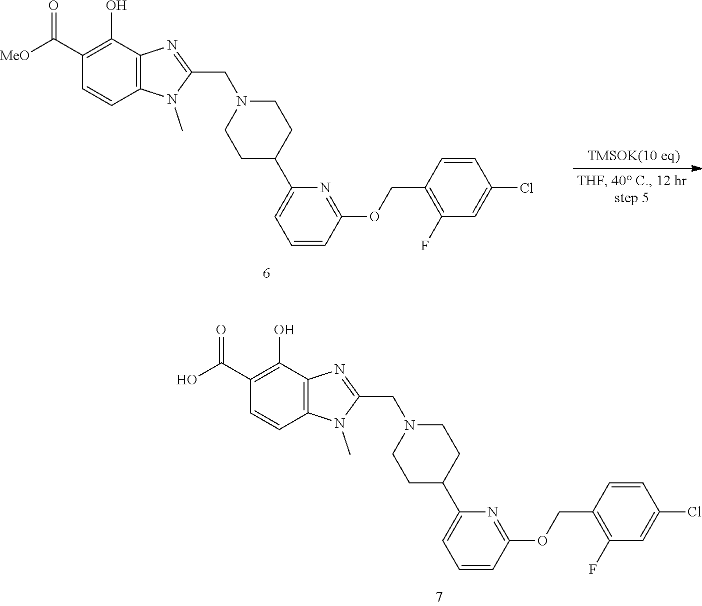 [SMILES: COC(=O)c1ccc2c(nc(CN3CCC(c4cccc(OCc5ccc(Cl)cc5F)n4)CC3)n2C)c1O ; Cn1c(CN2CCC(c3cccc(OCc4ccc(Cl)cc4F)n3)CC2)nc2c(O)c(C(=O)O)ccc21]

To a solution of 6 (80 mg, 148.42 μmol, 1 eq) in THF (1 mL) was added TMSOK (190.41 mg, 1.48 mmol, 10 eq). The mixture was stirred at 40° C. for 12 hr. LCMS (product: RT=1.201 min; start material: RT=1.688 min) showed ˜ 29% of the starting material was remained. The crude product was purified by reversed-phase HPLC (column: Waters Xbridge Prep OBD C18 150*40 mm*10 um; mobile phase: [H₂O (10 mM NH₄HCO₃)-ACN]; gradient: 25%-75% B over 8.0 min). 2-((4-(6-((4-Chloro-2-fluorobenzyl)oxy)pyridin-2-yl)piperidin-1-yl)methyl)-4-hydroxy-1-methyl-1H-benzo[d]imidazole-5-carboxylic acid (27.38 mg, 52.16 μmol, 35.14% yield) was obtained as a white solid.

LCMS: RT=2.766 min, MS cal.: 524.98, [M+H]⁺=525.2

HPLC: RT=11.320 min, purity=96.267%

¹H NMR (400 MHz, DMSO-d₆) δ=7.66-7.59 (m, 2H), 7.56 (t, J=8.0 Hz, 1H), 7.46 (br d, J=9.6 Hz, 1H), 7.29 (br d, J=8.4 Hz, 1H), 7.23-7.02 (m, 1H), 6.87 (d, J=7.2 Hz, 1H), 6.85-6.79 (m, 1H), 6.67 (d, J=8.4 Hz, 1H), 6.31-6.31 (m, 1H), 5.36 (s, 2H), 3.89 (br s, 2H), 3.82 (s, 3H), 3.07-2.97 (m, 2H), 2.70-2.59 (m, 1H), 2.39-2.25 (m, 2H), 1.88-1.69 (m, 4H)

### Example A85—Preparation of Compound 346 and Compound 347. 2-((4-(6-((4-Chloro-2-fluorobenzyl)oxy)pyridin-2-yl)piperidin-1-yl)methyl)-4-(fluoromethoxy)-1-(fluoromethyl)-1H-benzo[d]imidazole-6-carboxylic acid (Compound 346) (12) & 2-((4-(6-((4-Chloro-2-fluorobenzyl)oxy)pyridin-2-yl)piperidin-1-yl)methyl)-7-(fluoromethoxy)-1-(fluoromethyl)-1H-benzo[d]imidazole-5-carboxylic acid (Compound 347) (12A)

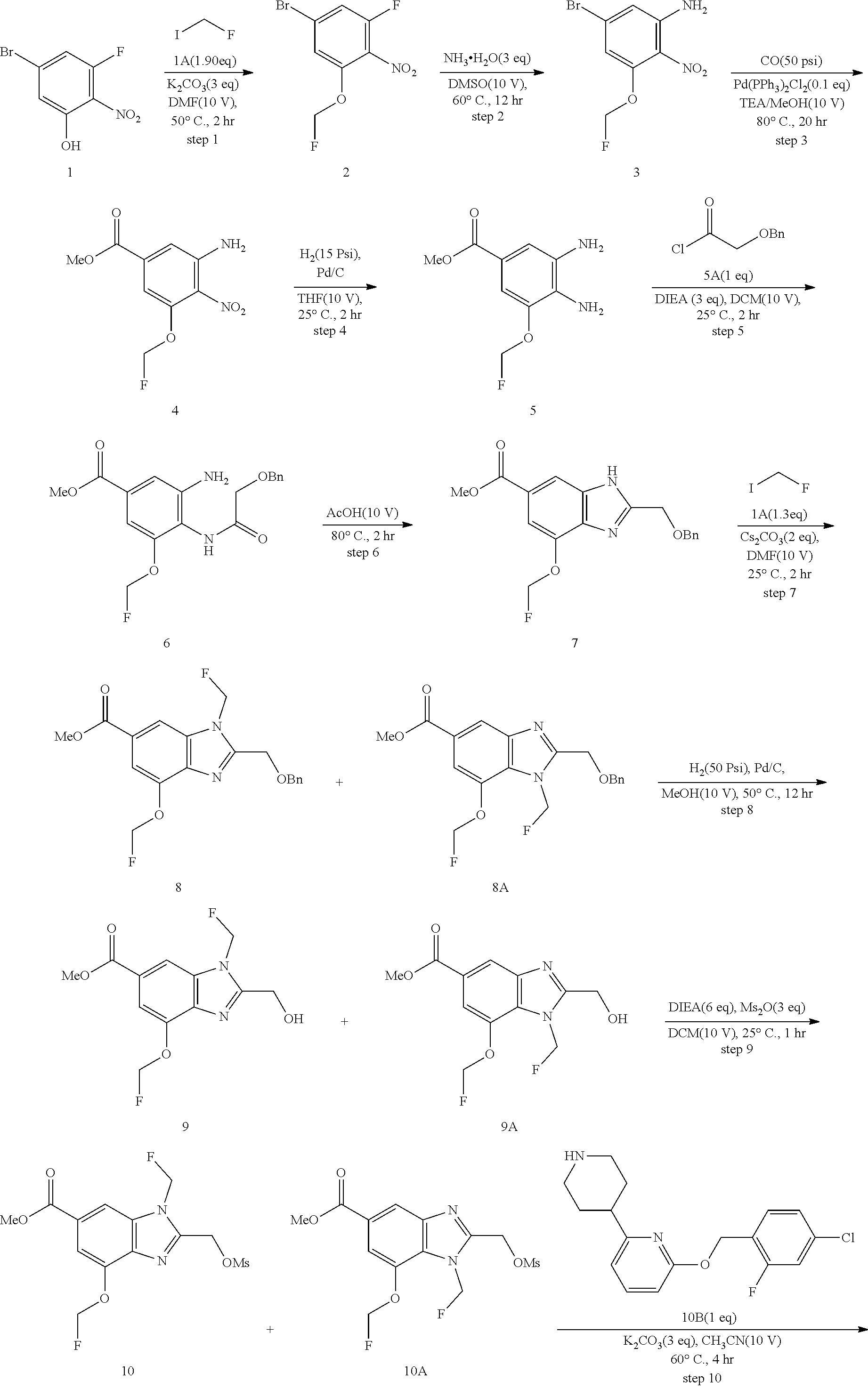

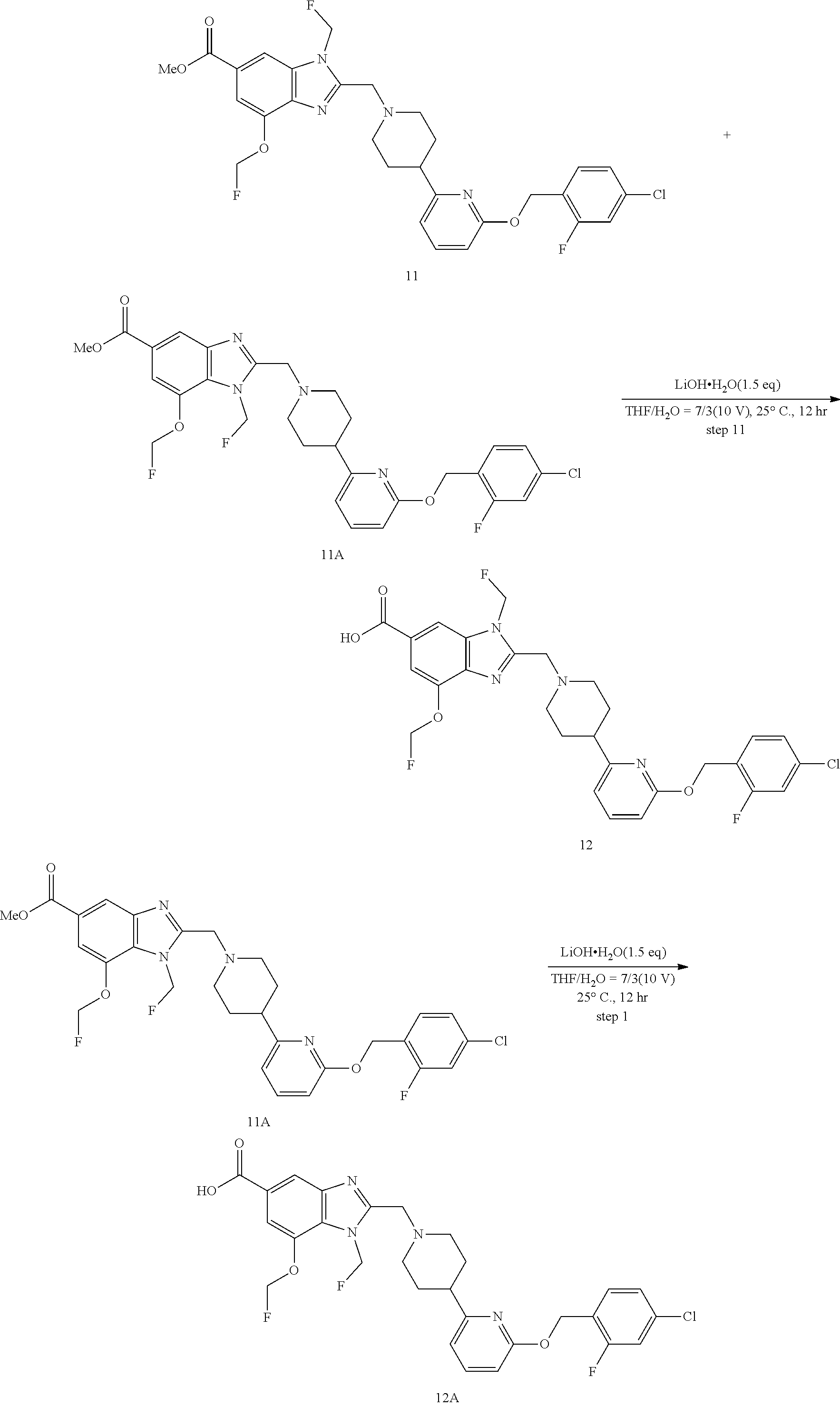 [SMILES: COC(=O)c1cc(OCF)c2nc(CN3CCC(c4cccc(OCc5ccc(Cl)cc5F)n4)CC3)n(CF)c2c1 ; COC(=O)c1cc(OCF)c2c(c1)nc(CN1CCC(c3cccc(OCc4ccc(Cl)cc4F)n3)CC1)n2CF ; O=C(O)c1cc(OCF)c2nc(CN3CCC(c4cccc(OCc5ccc(Cl)cc5F)n4)CC3)n(CF)c2c1 ; COC(=O)c1cc(OCF)c2c(c1)nc(CN1CCC(c3cccc(OCc4ccc(Cl)cc4F)n3)CC1)n2CF ; O=C(O)c1cc(OCF)c2c(c1)nc(CN1CCC(c3cccc(OCc4ccc(Cl)cc4F)n3)CC1)n2CF]

### General Procedure for Preparation of Intermediate 2:

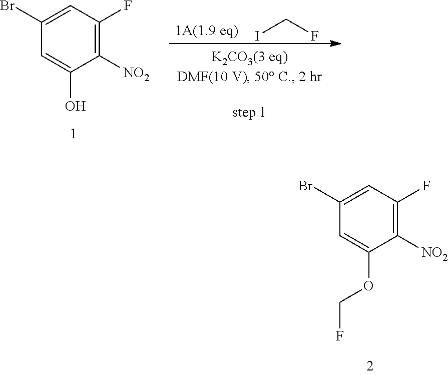

To a solution of 1 (1.5 g*2, 6.36 mmol, 1 eq) in DMF (30 mL) was added K₂CO₃ (2.64 g*2, 19.07 mmol, 3 eq) and 1A (1.93 g*2, 12.08 mmol, 1.9 eq) at 25° C. The mixture was stirred at 50° C. for 2 hr. TLC (Petroleum ether:Ethyl acetate-3:1) showed 1 was consumed completely. Two batches crude product mixture was combined for workup. The reaction mixture was diluted with H₂O (50 mL) and extracted with EtOAc (40 mL). The combined organic layers were washed with brine (40 mL), dried over Na₂SO₄, filtered and concentrated under reduced pressure to give 2 (3.2 g, 11.94 mmol, 93.92% yield) as a white solid.

LCMS: Rt=1.221 min, MS cal.: 266.93, [M−H]⁻=265.8

### General Procedure for Preparation of Intermediate 3:

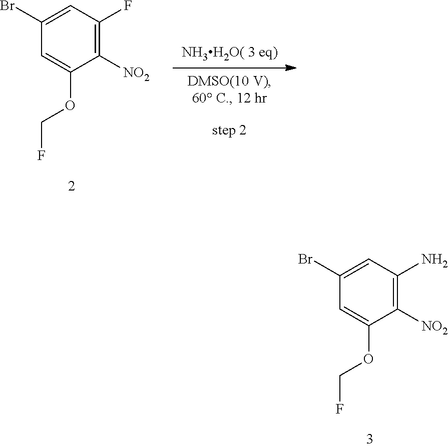 [SMILES: O=[N+]([O-])c1c(F)cc(Br)cc1OCF ; Nc1cc(Br)cc(OCF)c1[N+](=O)[O-]] [2886] To a solution of 2 (500 mg, 1.87 mmol, 1 eq) in DMSO (5 mL) was added NH₃·H₂O (784.57 mg, 5.60 mmol, 862.17 μL, 25% purity, 3 eq) at 25° C. The mixture was stirred at 60° C. for 12 hr. TLC (Petroleum ether:Ethyl acetate=3:1) showed 2 was consumed completely. The reaction mixture was diluted with H₂O (20 mL) and extracted with EtOAc (15 mL). The combined organic layers were washed with brine (20 mL), dried over Na₂SO₄, filtered and concentrated under reduced pressure to give 3 (450 mg, 1.70 mmol, 91.01% yield) as a yellow oil.

LCMS: Rt=1.094 min, MS cal.: 263.95, [M−H]⁻=262.8

¹H NMR (400 MHz, DMSO-d6) δ=6.85 (d, J=2.0 Hz, 1H), 6.65 (s, 1H), 6.45 (s, 2H), 5.94-5.81 (d, J=52.8 Hz, 2H),

### General Procedure for Preparation of Intermediate 4:

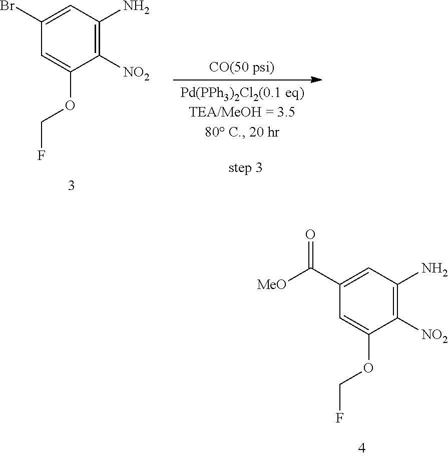 [SMILES: Nc1cc(Br)cc(OCF)c1[N+](=O)[O-] ; COC(=O)c1cc(N)c([N+](=O)[O-])c(OCF)c1]

To a solution of 3 (1.6 g, 6.04 mmol, 1 eq) in TEA (6 mL) and MeOH (10 mL) was added Pd(PPh₃)₂Cl₂ (423.73 mg, 603.69 μmol, 0.1 eq) at 25° C. After the addition, the mixture was stirred at 80° C. for 20 hr under CO (50 psi). TLC (Petroleum ether:Ethyl acetate=3:1) indicated 3 was consumed completely. The reaction mixture was filtered. The filtrate was diluted with H₂O (30 mL) and extracted with EtOAc (10 mL*3). The combined organic phase was washed with brine (10 mL*2), dried over Na₂SO₄, filtered and concentrated under reduced pressure to give a residue. The residue was purified by column chromatography (SiO₂, Petroleum ether/Ethyl acetate=1/0 to 0/1). 4 (900 mg, 3.69 mmol, 61.06% yield) was obtained as a yellow solid.

LCMS: Rt=0.431 min, MS cal.: 224.05, [M+H]⁻=245.0

### General Procedure for Preparation of Intermediate 5:

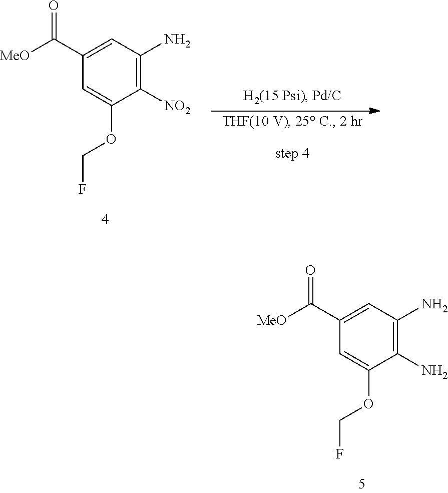 [SMILES: COC(=O)c1cc(N)c([N+](=O)[O-])c(OCF)c1 ; COC(=O)c1cc(N)c(N)c(OCF)c1]

THF (10 mL) was charged to the round-neck flask. 4 (0.94 g, 3.85 mmol, 1 eq) was added at 25° C. Pd/C (5.00 g) was added to the reaction mixture at 25° C. After the addition, the mixture was stirred at 25° C. for 2 hr. LC-MS showed 4 was consumed completely and desired mass was detected. The reaction mixture was filtered through celite pad. The filter cake was rinsed with MeOH (30 ml*3), and the filtrate was concentrated. 5 (810 mg, 3.78 mmol, 98.23% yield) was obtained as a yellow solid.

LCMS: RT=0.831 min, MS cal.: 214.08, [M+H]⁻=214.9

1H NMR (400 MHz, DMSO-d6) δ=7.10-6.95 (m, 2H), 5.85-5.60 (d, J=55.2 Hz, 2H), 5.08 (br s, 2H), 4.93 (br s, 2H), 3.73 (s, 3H)

### General Procedure for Preparation of Intermediate 6:

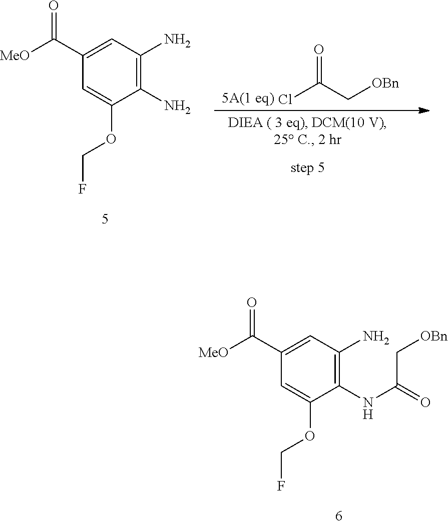 [SMILES: COC(=O)c1cc(N)c(N)c(OCF)c1 ; O=C(Cl)COCc1ccccc1 ; COC(=O)c1cc(N)c(NC(=O)COCc2ccccc2)c(OCF)c1]

To a solution of 5 (800 mg, 3.73 mmol, 1 eq), 5A (689.54 mg, 3.73 mmol, 579.93 μL, 1 eq) in DCM (8 mL) was added DIEA (1.45 g, 11.20 mmol, 1.95 mL, 3 eq). The mixture was stirred at 25° C. for 2 hr. LC-MS showed 5 was consumed completely and desired mass was detected. The reaction mixture was diluted with H₂O (20 mL), was extracted with EtOAc (10 mL*3). The combined organic phase was washed with brine (10 mL*2), dried over Na₂SO₄, filtered, and concentrated under reduced pressure to give a residue. The residue was purified by column chromatography (SiO₂, Petroleum ether/Ethyl acetate=4/1 to 1/1). 6 (800 mg, 2.21 mmol, 59.11% yield) was obtained as a yellow solid.

LCMS: RT=1.098 min, MS cal.: 362.35, [M+H]⁺=363.1

¹H NMR (400 MHz, DMSO-d₆) δ=9.27 (s, 1H), 7.69 (s, 1H), 7.50-7.27 (m, 6H), 5.90-5.76 (d, J=54.4 Hz, 2H), 5.62 (br s, 2H), 4.64 (s, 2H), 4.16 (s, 2H), 3.78 (s, 3H)

### General Procedure for Preparation of Intermediate 7:

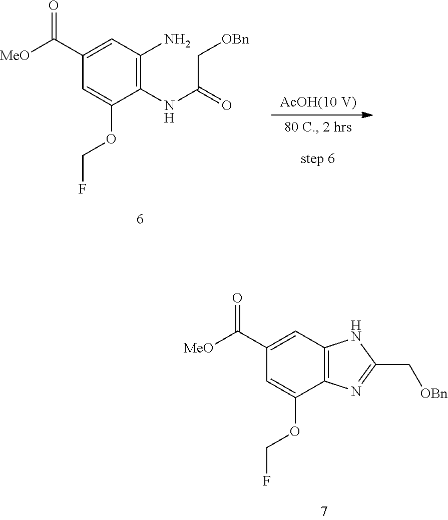 [SMILES: COC(=O)c1cc(N)c(NC(=O)COCc2ccccc2)c(OCF)c1 ; COC(=O)c1cc(OCF)c2nc(COCc3ccccc3)[nH]c2c1]

To a solution of 6 (800 mg, 2.21 mmol, 1 eq) in AcOH (9 mL). The mixture was stirred at 80° C. for 2 hr. LC-MS showed 6 was consumed completely and desired mass was detected. The residue was diluted with H₂O (20 mL) and extracted with EtOAc (30 mL). The combined organic layers were washed with brine (20 mL), dried over Na₂SO₄ filtered and concentrated under reduced pressure to give 7 (890 mg, 2.20 mmol, 99.69% yield) was obtained as an orange oil.

LCMS: RT=1.125 min, MS cal.: 404.37, [M+H]⁺=345.1

### General Procedure for Preparation of Intermediate 8 & 8A:

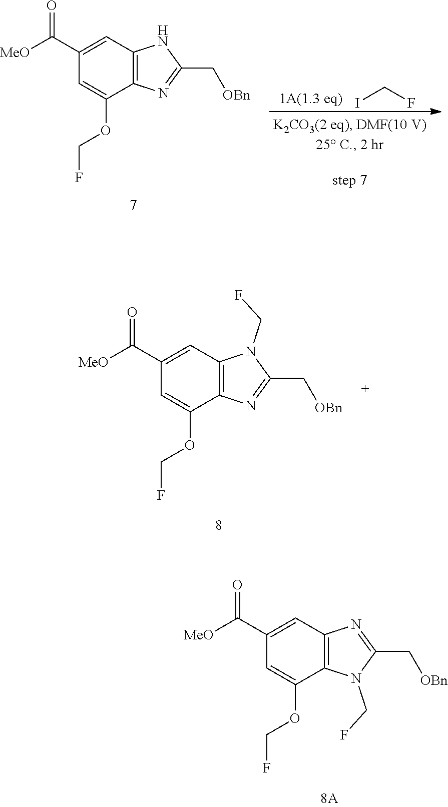

To a solution of 7 (500 mg, 1.45 mmol, 1 eq) in DMF (5 mL) was added Cs₂CO₃ (946.23 mg, 2.90 mmol, 2 eq) and 1A (301.90 mg, 1.89 mmol, 1.3 eq) at 25° C. The mixture was stirred at 25° C. for 2 hr. LC-MS showed 7 was consumed completely and desired mass was detected. The reaction mixture was diluted with H₂O (20 mL) and extracted with EtOAc (30 mL). The combined organic layers were washed with brine (20 mL), dried over Na₂SO₄ filtered and concentrated under reduced pressure to give 8 & 8A (500 mg, crude in total) was obtained as a yellow solid.

LCMS: RT=7.957 min, MS cal.: 376.38, [M+H]⁺=377.2

### General Procedure for Preparation of Intermediate 9 & Intermediate 9A:

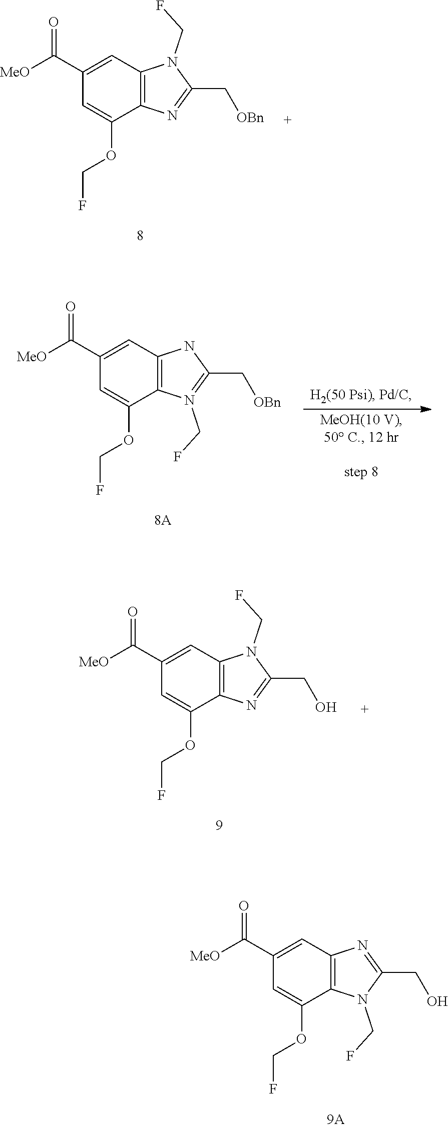

MeOH (5 mL) was charged to the round bottom flask, then Pd/C (250 mg, 234.92 μmol, 10% purity) was added to the mixture at 25° C. 8 & 8A (480 mg, crude) was added to the reaction mixture. After the addition, the mixture was stirred at 50° C. for 12 hr. LCMS showed 8 & 8A was consumed completely and desired mass was detected. The reaction mixture was filtered through celite pad. The filter cake was rinsed with MeOH (30 ml*3), and the filtrate was concentrated under reduced pressure to give 9 & 9A (300 mg, crude in total) as a white solid.

LCMS: RT=0.890 min, MS cal.: 286.23, [M+H]⁺=268.9

### General Procedure for Preparation of Intermediate 10 & Intermediate 10A:

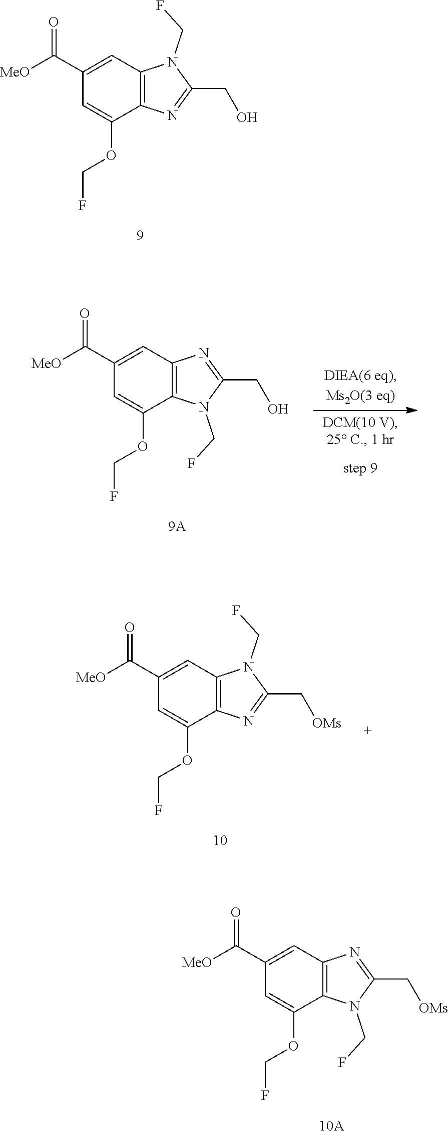 [SMILES: COC(=O)c1cc(OCF)c2nc(CO)n(CF)c2c1 ; COC(=O)c1cc(OCF)c2c(c1)nc(CO)n2CF ; COC(=O)c1cc(OCF)c2nc(COS(C)(=O)=O)n(CF)c2c1 ; COC(=O)c1cc(OCF)c2c(c1)nc(COS(C)(=O)=O)n2CF]

To a solution of 9 & 9A (250 mg, crude) in DCM (3 mL) was added DIEA (677.30 mg, 5.24 mmol, 912.80 μL, 6 eq) and Ms₂O (456.44 mg, 2.62 mmol, 3 eq). The mixture was stirred at 25° C. for 1 hr. LCMS showed 9 & 9A was consumed completely and desired mass was detected. The reaction mixture was diluted with H₂O (10 mL) and extracted with DCM (15 mL). The combined organic layers were washed with brine (10 mL), dried over Na₂SO₄, filtered, and concentrated under reduced pressure to give 10 & 10A (318 mg, crude in total) as a yellow oil.

LCMS: RT=1.025 min, MS cal.: 364.32, [M+H]⁺=365.0

### General Procedure for Preparation of Intermediate 11 & Intermediate 11A:

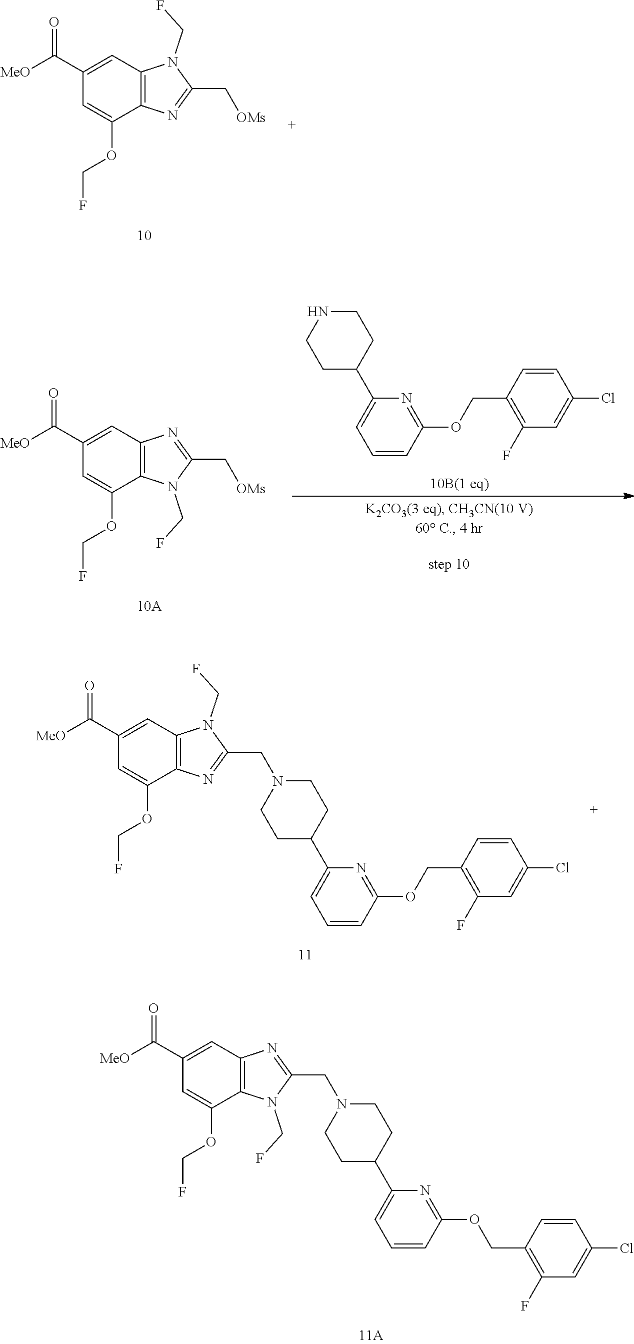 [SMILES: COC(=O)c1cc(OCF)c2nc(COS(C)(=O)=O)n(CF)c2c1 ; COC(=O)c1cc(OCF)c2c(c1)nc(COS(C)(=O)=O)n2CF ; Fc1cc(Cl)ccc1COc1cccc(C2CCNCC2)n1 ; COC(=O)c1cc(OCF)c2nc(CN3CCC(c4cccc(OCc5ccc(Cl)cc5F)n4)CC3)n(CF)c2c1 ; COC(=O)c1cc(OCF)c2c(c1)nc(CN1CCC(c3cccc(OCc4ccc(Cl)cc4F)n3)CC1)n2CF]

To a solution of 10 & 10A (318 mg, crude), 10B (280.00 mg, 872.86 μmol, 1 eq) in CH₃CN (4 mL) was added K₂CO₃ (361.90 mg, 2.62 mmol, 3 eq). The mixture was stirred at 60° C. for 4 hr. LC-MS showed 10 & 10A was consumed completely and desired mass was detected. The residue was diluted with H₂O (20 mL) and extracted with EtOAc (30 mL). The combined organic layers were washed with brine (20 mL), dried over Na₂SO₄ filtered and concentrated under reduced pressure to give a residue. The residue was purified by column chromatography (SiO₂, Petroleum ether/Ethyl acetate=2/1 to 0/1). 11 & 11A (245 mg, crude in total) was obtained as a yellow solid.

LCMS: RT=1.692 min, MS cal.: 589.0, [M+H]⁺=589.2

### General Procedure for Preparation of Intermediate 11 & Intermediate 11A:

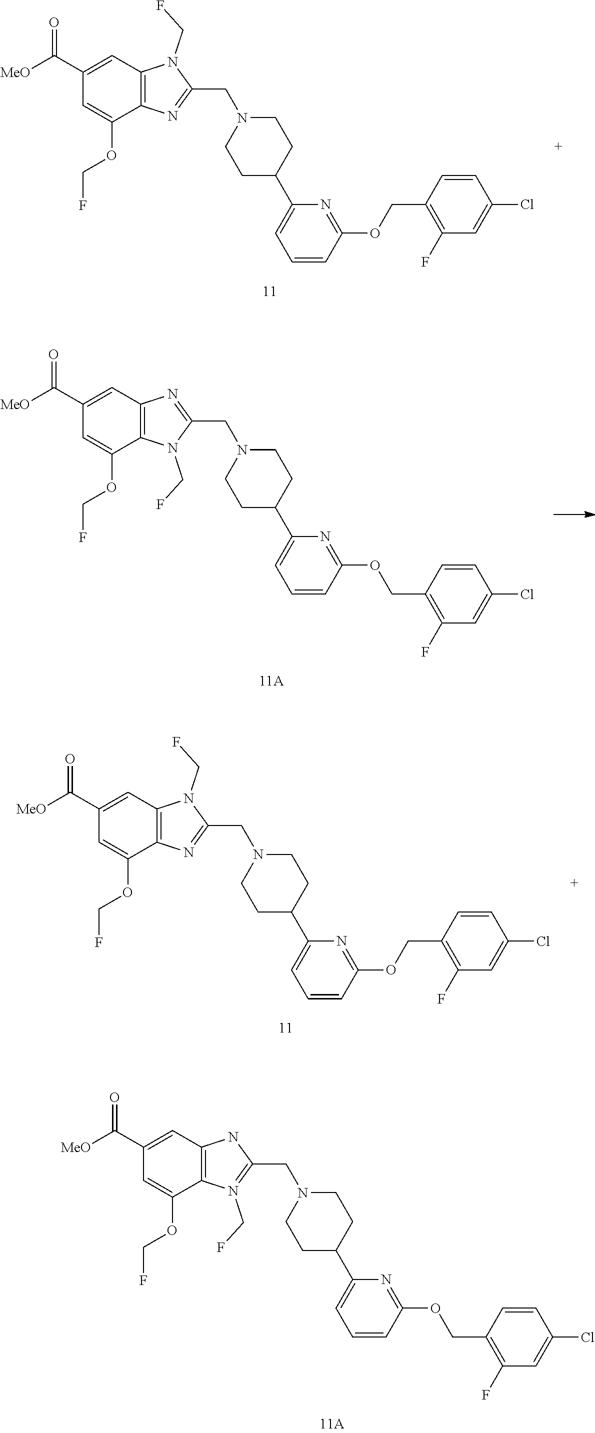

The residue was purified by prep-SFC (column: REGIS (s,s) WHELK-01 (250 mm 30 mm, 5 um); mobile phase: [CO₂-MeOH]; B %: 50%, isocratic elution mode). 11 (100 mg, 169.78 μmol, 40.82% yield) and 11A (106 mg, 179.96 μmol, 43.27% yield) were obtained as a yellow solid.

¹H NMR (400 MHz, DMSO-d₆) (Compound 11) δ=8.16 (s, 1H), 7.67-7.52 (m, 3H), 7.45 (dd, J=1.6, 10.0 Hz, 1H), 7.33-7.24 (m, 1H), 6.86 (d, J=7.2 Hz, 1H), 6.73-6.60 (d, J=52.0 Hz, 1H), 6.66 (d, J=8.2 Hz, 1H), 6.22-6.03 (d, J=53.6 Hz, 2H), 5.36 (s, 2H), 3.94 (s, 2H), 3.91 (s, 3H), 2.93 (br d, J=11.2 Hz, 2H), 2.62-2.56 (m, 1H), 2.21 (br t, J=10.4 Hz, 2H), 1.84-1.65 (m, 4H)

¹H NMR (400 MHz, DMSO-d₆) (Compound 11A) δ=8.03 (d, J=1.2 Hz, 1H), 7.69 (s, 1H), 7.64-7.59 (m, 1H), 7.57 (t, J=8.2 Hz, 1H), 7.45 (dd, J=2.0, 10.0 Hz, 1H), 7.28 (dd, J=1.8, 8.2 Hz, 1H), 6.85 (d, J=7.3 Hz, 1H), 6.69-6.56 (d, J=52.0 Hz, 2H), 6.66 (d, J=8.1 Hz, 1H), 6.17-6.00 (d, J=53.6 Hz, 2H), 5.35 (s, 2H), 3.92 (s, 2H), 3.89 (s, 3H), 2.93 (br d, J=11.2 Hz, 2H), 2.63-2.54 (m, 1H), 2.26-2.15 (m, 2H), 1.84-1.66 (m, 4H)

### General Procedure for Preparation of Compound 346 (12):

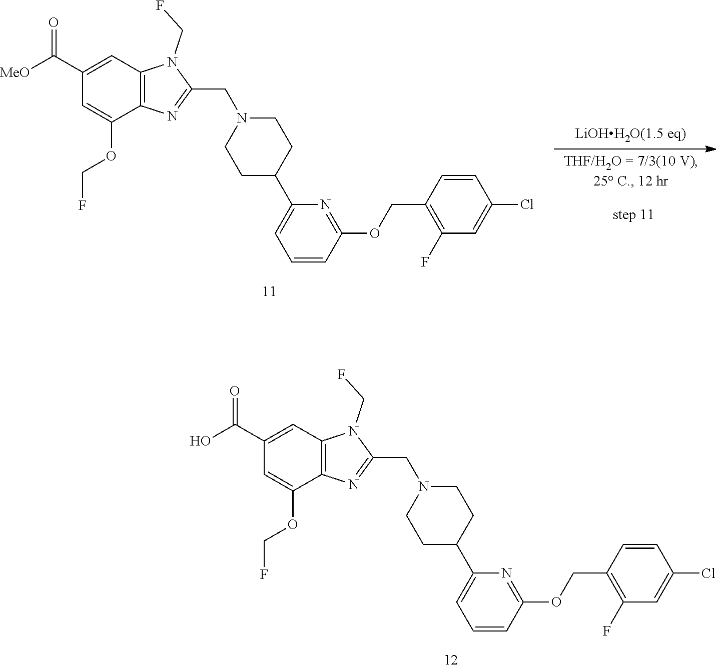 [SMILES: COC(=O)c1cc(OCF)c2nc(CN3CCC(c4cccc(OCc5ccc(Cl)cc5F)n4)CC3)n(CF)c2c1 ; O=C(O)c1cc(OCF)c2nc(CN3CCC(c4cccc(OCc5ccc(Cl)cc5F)n4)CC3)n(CF)c2c1]

To a solution of 11 (90 mg, 152.80 μmol, 1 eq) in THF (0.7 mL) was added LiOH·H₂O (9.62 mg, 229.20 μmol, 1.5 eq) in H₂O (0.3 mL). The mixture was stirred at 25° C. for 12 hr. LC-MS showed 11 was consumed completely and desired mass was detected. The reaction mixture was purified directly. The residue was purified by prep-HPLC (neutral condition; column: Waters Xbridge Prep OBD C18 150*40 mm*10 um; mobile phase: [H₂O (10 mM NH₄HCO₃)-ACN]; gradient: 30%-60% B over 8.0 min). 2-((4-(6-((4-Chloro-2-fluorobenzyl)oxy)pyridin-2-yl)piperidin-1-yl)methyl)-4-(fluoromethoxy)-1-(fluoromethyl)-1H-benzo[d]imidazole-6-carboxylic acid (28.32 mg, 49.25 μmol, 32.23% yield) was obtained as a white solid.

LCMS: RT=2.783 min, MS cal.: 574.98, [M+H]⁺=575.2

HPLC: RT=11.896 min, purity: 99.7%

¹H NMR (400 MHz, MeOH-d4) δ=8.10 (s, 1H), 7.75 (s, 1H), 7.57 (t, J=7.6 Hz, 1H), 7.50 (t, J=8.0 Hz, 1H), 7.24-7.16 (m, 2H), 6.82 (d, J=7.2 Hz, 1H), 6.67-6.54 (d, J=52.4 Hz, 2H), 6.62 (d, J=8.0 Hz, 1H), 6.07-5.94 (d, J=54.0 Hz, 2H), 5.40 (s, 2H), 4.00 (s, 2H), 3.02 (br d, J=11.6 Hz, 2H), 2.70-2.59 (m, 1H), 2.38-2.27 (m, 2H), 1.96-1.82 (m, 4H)

### General Procedure for Preparation of Compound 347 (12A):

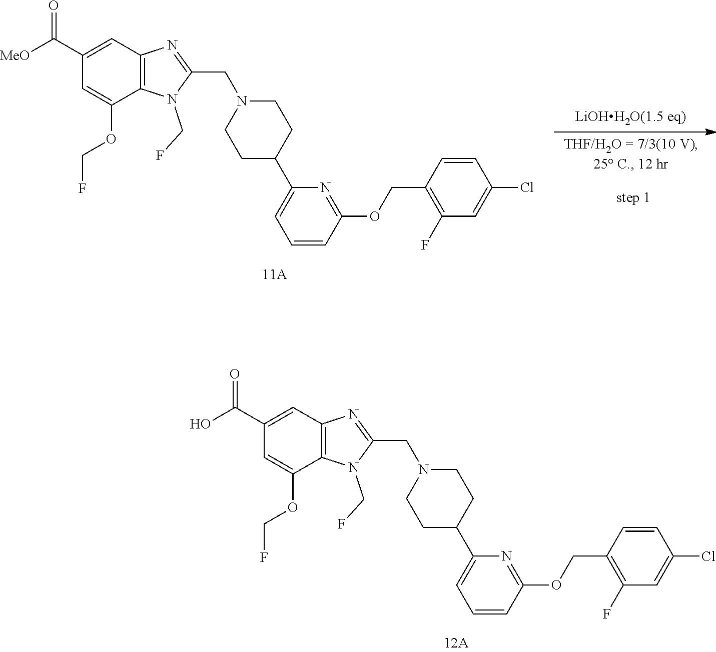 [SMILES: COC(=O)c1cc(OCF)c2c(c1)nc(CN1CCC(c3cccc(OCc4ccc(Cl)cc4F)n3)CC1)n2CF ; O=C(O)c1cc(OCF)c2c(c1)nc(CN1CCC(c3cccc(OCc4ccc(Cl)cc4F)n3)CC1)n2CF]

A mixture of 11A (90.00 mg, 152.80 μmol, 1 eq), LiOH·H₂O (9.62 mg, 229.20 μmol, 1.5 eq) in THF (0.7 mL) and H₂O (0.3 mL) was degassed and purged with N₂ for 3 times, and then the mixture was stirred at 25° C. for 12 hrs under N₂ atmosphere. LC-MS showed 11A was consumed completely and one main peak with desired mass was detected. The reaction mixture was diluted with ACN 0.5 mL. The mixture was purified by reversed-phase HPLC (column: Waters Xbridge Prep OBD C₁₈ 150*40 mm*10 um; mobile phase: [H₂O (10 mM NH₄HCO₃)-ACN]; gradient: 20%-70% B over 8.0 min). 2-((4-(6-((4-Chloro-2-fluorobenzyl)oxy)pyridin-2-yl)piperidin-1-yl)methyl)-7-(fluoromethoxy)-1-(fluoromethyl)-1H-benzo[d]imidazole-5-carboxylic acid (29.07 mg, 50.56 μmol, 33.09% yield) was obtained as a white solid.

LCMS of ET62904-747-p1a1: RT=0.449 min, MS cal.: 574.16, [M+H]⁺=575.1

HPLC: RT=11.866 min, purity: 100.00%

¹H NMR (400 MHz, MeOH-d4) δ=8.13 (d, J=1.2 Hz, 1H), 7.83 (s, 1H), 7.61-7.45 (m, 2H), 7.25-7.13 (m, 2H), 6.82 (d, J=7.2 Hz, 1H), 6.79-6.64 (d, J=51.6 Hz, 2H), 6.62 (d, J=8.4 Hz, 1H), 6.05-5.92 (d, J=53.6 Hz, 2H), 5.40 (s, 2H), 3.97 (s, 2H), 3.02 (br d, J=11.2 Hz, 2H), 2.73-2.58 (m, 1H), 2.32 (dt, J=4.4, 10.8 Hz, 2H), 1.94-1.82 (m, 4H)

### Example A86—Preparation of Compound 348. 2-((4-(6-((4-Chloro-2-methoxybenzyl)oxy)pyridin-2-yl)piperidin-1-yl)methyl)-4-methoxy-1-methyl-1H-benzo[d]imidazole-6-carboxylic acid (Compound 348) (9)

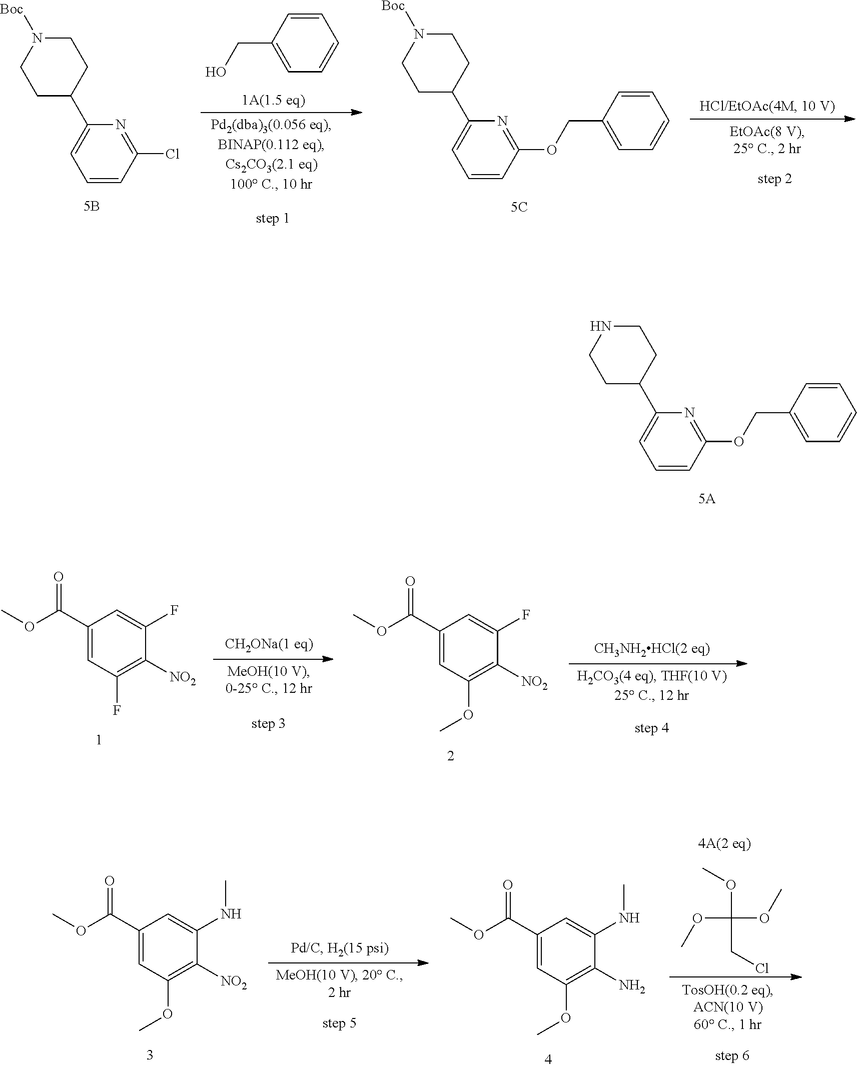

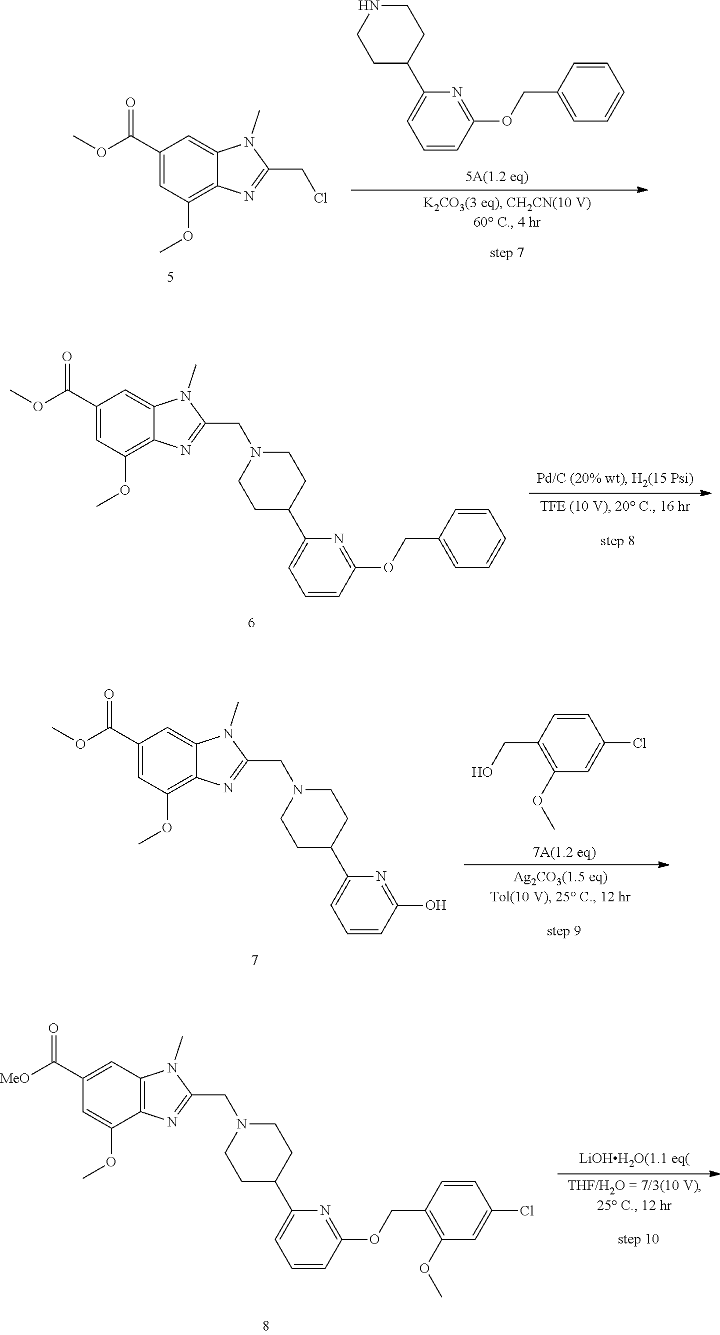 [SMILES: COC(=O)c1cc(OC)c2nc(CCl)n(C)c2c1 ; c1ccc(COc2cccc(C3CCNCC3)n2)cc1 ; COC(=O)c1cc(OC)c2nc(CN3CCC(c4cccc(OCc5ccccc5)n4)CC3)n(C)c2c1 ; COC(=O)c1cc(OC)c2nc(CN3CCC(c4cccc(O)n4)CC3)n(C)c2c1 ; COc1cc(Cl)ccc1CO ; COC(=O)c1cc(OC)c2nc(CN3CCC(c4cccc(OCc5ccc(Cl)cc5OC)n4)CC3)n(C)c2c1]

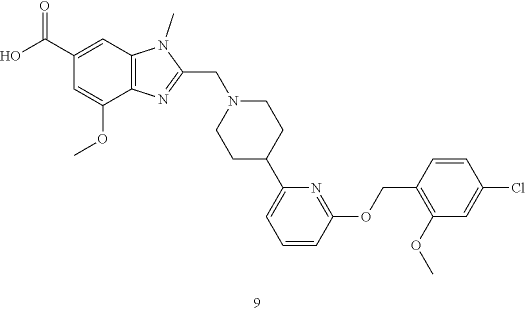 [SMILES: COc1cc(Cl)ccc1COc1cccc(C2CCN(Cc3nc4c(OC)cc(C(=O)O)cc4n3C)CC2)n1]

### General Procedure for Preparation of Intermediate 5C:

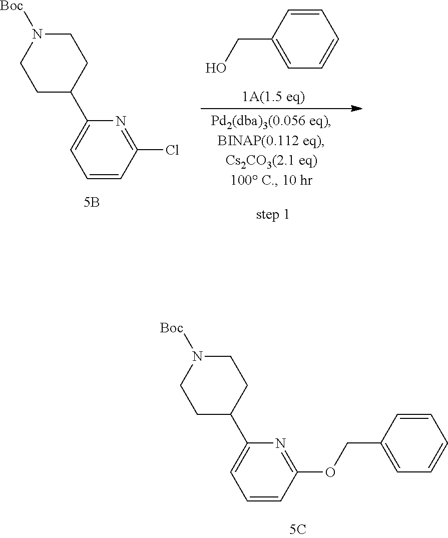 [SMILES: CC(C)(C)OC(=O)N1CCC(c2cccc(Cl)n2)CC1 ; OCc1ccccc1 ; CC(C)(C)OC(=O)N1CCC(c2cccc(OCc3ccccc3)n2)CC1]

A mixture of 5B (40 g, 134.77 mmol, 1 eq), 1A (21.86 g, 202.16 mmol, 20.94 mL, 1.5 eq), Pd₂ (dba) 3 (6.91 g, 7.55 mmol, 0.056 eq), Cs₂CO₃ (92.22 g, 283.03 mmol, 2.1 eq) and BINAP (9.40 g, 15.09 mmol, 0.112 eq) in dioxane (400 mL) was degassed and purged with N₂ for 3 times, and then the mixture was stirred at 100° C. for 10 hr under N₂ atmosphere. LCMS indicated Reactant 1 was consumed completely and one main peak with desired mass was detected. The reaction mixture was filtered, the filter was poured into water (1000 mL). The aqueous phase was extracted with EtOAc (500 mL*3). The combined organic phase was washed with brine (500 mL*2), dried with anhydrous Na₂SO₄, filtered and concentrated in vacuum. The residue was purified by column chromatography (SiO₂, Petroleum ether/Ethyl acetate=1/0 to 20/1). 5C (40 g, crude) was a yellow solid.

LCMS: RT=2.245 min, MS cal.: 398.5, [M+H]⁺=369.5

### General Procedure for Preparation of Intermediate 5A:

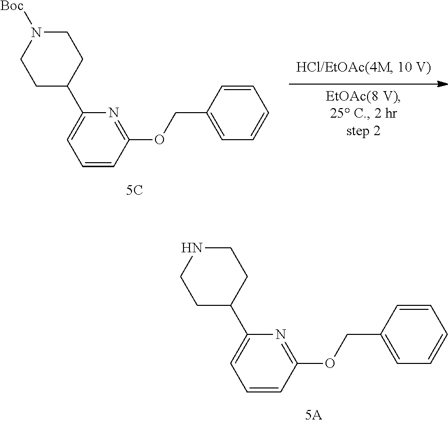 [SMILES: CC(C)(C)OC(=O)N1CCC(c2cccc(OCc3ccccc3)n2)CC1 ; "EtOAc(8 V),25° C., 2 hrstep 2" ; c1ccc(COc2cccc(C3CCNCC3)n2)cc1]

To a solution of 5C (17.00 g, 46.14 mmol, 1 eq) in EtOAc (150 mL) was added HCl/EtOAc (4 M, 155.35 mL, 13.47 eq). The mixture was stirred at 25° C. for 4 hr. TLC indicated Reactant 1 was consumed completely and one new spot formed. The reaction was clean according to TLC. The reaction mixture was filtered and filter cake was concentrated under reduced pressure. 5A (80 g, 213.85 mmol, 92.70% yield, 2.9 HCl) was a colorless oil.

LCMS: RT=1.190 min, MS cal.: 268.1, [M+H]⁺=269.1

¹H NMR (400 MHz, DEUTERIUM OXIDE) δ=8.30 (t, J=8.4 Hz, 1H), 7.52-7.28 (m, 7H), 5.46 (s, 2H), 3.54 (br d, J=13.2 Hz, 2H), 3.24-3.07 (m, 3H), 2.20 (br d, J=14.4 Hz, 2H), 2.04-1.90 (m, 2H)

### General Procedure for Preparation of Intermediate 2:

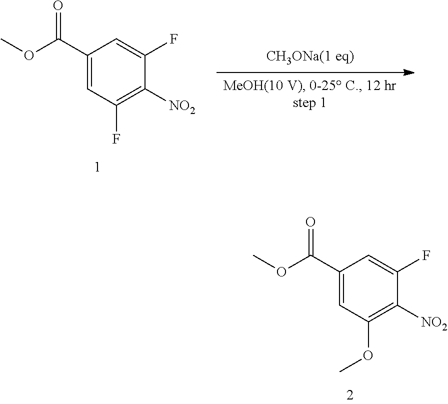 [SMILES: COC(=O)c1cc(F)c([N+](=O)[O-])c(F)c1 ; "MeOH(10 V), 0-25° C., 12 hrstep 1" ; COC(=O)c1cc(F)c([N+](=O)[O-])c(OC)c1]

To a solution of 1 (20 g, 92.11 mmol, 1 eq) in MeOH (200 mL) was added CH₃ONa (4.98 g, 92.11 mmol, 1 eq) in portions at 0° C. The mixture was stirred at 25° C. for 12 hr. TLC (Petroleum ether:Ethyl acetate=3:1, rt=0.47) indicated 1 was consumed completely and one new spot formed. The reaction was clean according to TLC. The reaction mixture was concentrated under reduced pressure to remove solvent to give a residue. The crude product was triturated with H₂O (200 ml) at 25° C. for 30 min. The filter cake is the product. 2 (20 g, 86.40 mmol, 93.80% yield, 99% purity) was obtained as a white solid.

¹H NMR (400 MHz, CHCl3-d) δ=7.54-7.47 (m, 2H), 3.96 (d, J=9.9 Hz, 6H)

### General Procedure for Preparation of Intermediate 3:

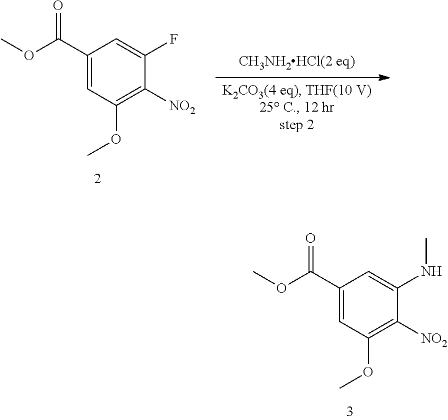 [SMILES: COC(=O)c1cc(F)c([N+](=O)[O-])c(OC)c1 ; "K2CO3(4 eq), THF(10 V)25° C., 12 hrstep 2" ; CNc1cc(C(=O)OC)cc(OC)c1[N+](=O)[O-]]

Equip a 1000 mL three-necked round bottom flask, addition funnel and thermometer, N₂ balloon etc. THF (300 mL) was charged to the 1000 mL three-necked round bottom flask, then 2 (30 g, 130.91 mmol, 1 eq), K₂CO₃ (72.37 g, 523.65 mmol, 4 eq) was added to the mixture at 25° C. At 25° C., methanamine; hydrochloride (17.68 g, 261.82 mmol, 2 eq) was added dropwise to the reaction mixture at 25° C. within 10 min. After the addition, the mixture was stirred at 25° C. for 12 hr. HPLC showed 2 was consumed completely and one main peak with desired m/z was detected. The reaction mixture was added with FA (1 M) to adjust pH=5 at 25° C. The mixture was extracted by EtOAc (2000 mL). Then organic phase was combined and washed by H₂O (3000 mL), brine (200 mL), dried with Na₂SO₄, filtered. The organic phase was concentrated under reduced pressure at 45° C. to give a residue. 3 (31 g, 122.60 mmol, 93.65% yield, 95% purity) was obtained as a red solid.

¹H NMR (400 MHz, CHCl3-d) δ=7.08 (d, J=1.4 Hz, 1H), 6.96 (d, J=1.4 Hz, 1H), 3.96 (d, J=3.8 Hz, 7H), 2.96 (s, 3H)

### General Procedure for Preparation of Intermediate 4:

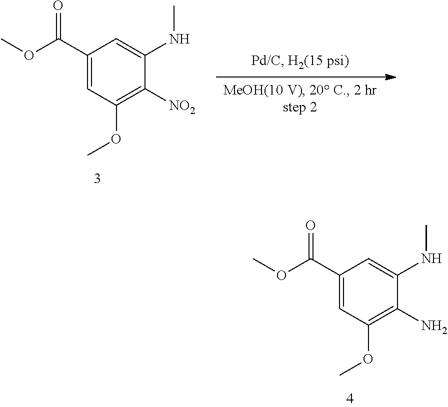

Equip a 2000 mL round bottom flask. MeOH (200 mL) was charged to the round bottom flask, then Pd/C (3.10 g) was added to the mixture at 20° C. At 20° C. (inner temperature), 3 (31 g, 129.05 mmol, 1 eq) in MeOH (110 mL) was added to the reaction mixture at 20° C. under N₂ atmosphere. After the addition, the suspension was degassed and purged with H₂ for 3 times, and then the mixture was stirred under H₂ (15 Psi) atmosphere at 20° C. for 2 hr. LC-MS showed 3 was consumed completely and one main peak with desired m/z was detected. The mixture is filtered with MeOH (1000 ml) and the filtrate is concentrated under reduced pressure. 4 (27 g, 115.59 mmol, 89.57% yield, 90% purity) was obtained as a brown oil.

LCMS: RT=0.286 min, MS cal.: 210.10, [M+H]⁺=211.10

¹H NMR (400 MHz, CHCl3-d) δ=7.16 (d, J=1.6 Hz, 1H), 7.16 (d, J=1.6 Hz, 1H), 3.92 (d, J=6.8 Hz, 6H), 2.92 (s, 3H)

### General Procedure for Preparation of Intermediate 5:

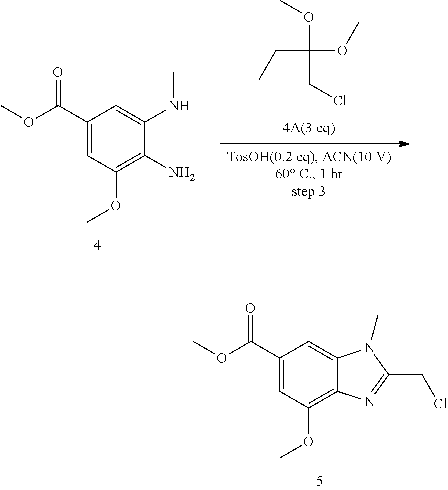

A mixture of 4 (25 g, 118.92 mmol, 1 eq), 4A (36.77 g, 237.84 mmol, 32.06 mL, 2 eq), TosOH (4.10 g, 23.78 mmol, 0.2 eq), in ACN (250 mL) was degassed and purged with N₂ for 3 times, and then the mixture was stirred at 60° C. for 1 hr under N₂ atmosphere. TLC indicated 4 was consumed completely and one new spot formed. The reaction was clean according to TLC. The reaction mixture was concentrated under reduced pressure to remove ACN. The residue was diluted with aq. NaHCO₃ (200 mL) and extracted with DCM (300 mL*3). The combined organic layers were washed with brine (300 mL), dried over Na₂SO₄, filtered and concentrated under reduced pressure to give a residue. The crude product was triturated with MTBE (3 V) at 25° C. for 1 h. 5 (26.8 g, 94.75 mmol, 79.68% yield, 95% purity) was obtained as a white solid.

¹H NMR (400 MHz, CHCl3-d) δ=7.76 (d, J=0.9 Hz, 1H), 7.40 (s, 1H), 4.88 (s, 2H), 4.08 (s, 3H), 3.96 (s, 4H), 3.96-3.88 (m, 3H)

### General Procedure for Preparation of Intermediate 6:

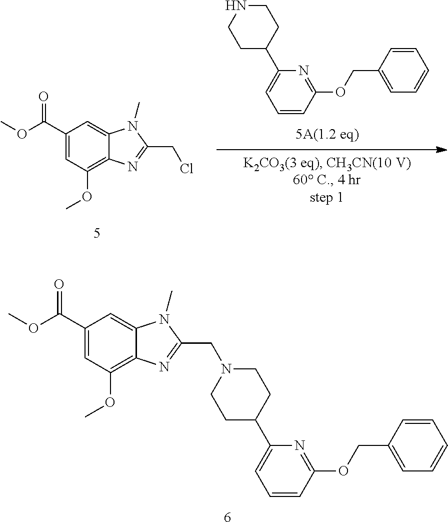

To a solution of 5 (4.99 g, 18.61 mmol, 1 eq) in CH₃CN (50 mL) was added K₂CO₃ (7.72 g, 55.83 mmol, 3 eq). The mixture was stirred at 60° C. for 12 hr. LCMS indicated Reactant 1 was consumed completely. Filtered and concentrated in vacuum. The residue was purified by column chromatography (SiO₂, Petroleum ether/Ethyl acetate=1/0 to 0/1). 6 (7.8 g, 15.58 mmol, 83.73% yield) was a brown solid.

LCMS: RT=1.527 min, MS cal.: 500.2, [M+H]⁺=501.3

¹H NMR (400 MHz, CHCl3-d) δ=7.79 (d, J=0.8 Hz, 1H), 7.52-7.44 (m, 3H), 7.40 (s, 1H), 7.38-7.34 (m, 2H), 7.33-7.28 (m, 1H), 6.72 (d, J=7.2 Hz, 1H), 6.61 (br d, J=8.0 Hz, 1H), 5.38 (s, 2H), 4.07 (s, 3H), 3.98 (br s, 2H), 3.97 (s, 3H), 3.89 (br s, 2H), 2.98 (br d, J=3.2 Hz, 2H), 2.71-2.55 (m, 1H), 2.37-2.22 (m, 2H), 1.96-1.75 (m, 4H)

### General Procedure for Preparation of Intermediate 7:

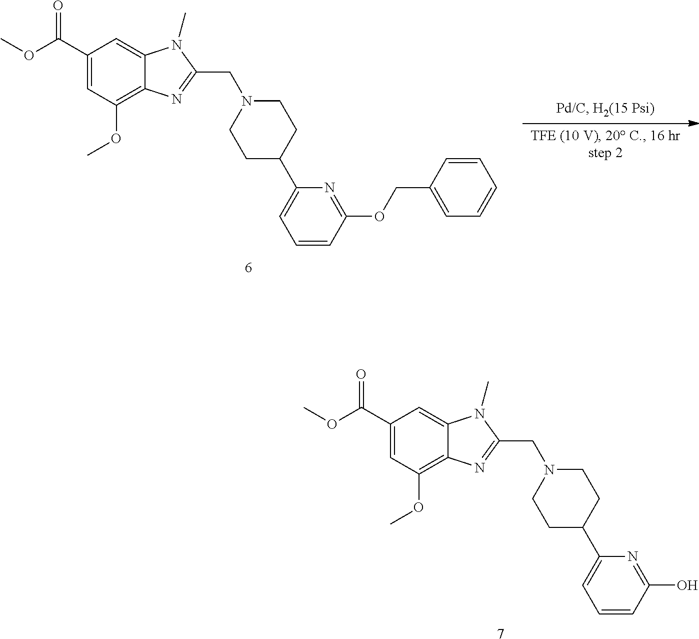 [SMILES: COC(=O)c1cc(OC)c2nc(CN3CCC(c4cccc(OCc5ccccc5)n4)CC3)n(C)c2c1 ; "Pd/C, H2(15 Psi)" ; "TFE (10 V), 20° C., 16 hrstep 2" ; "6" ; COC(=O)c1cc(OC)c2nc(CN3CCC(c4cccc(O)n4)CC3)n(C)c2c1 ; "7"]

Equip a 250 mL round bottom flask, addition funnel N₂ balloon. TFE (70 mL) was charged to the one-necked round bottom flask, then 6 (7.3 g, 14.58 mmol, 1 eq) was added to the mixture at 15° C. Pd/C (1.55 g) was added to the reaction mixture at 15° C. After the addition, the mixture was degassed and purged with H₂ for 3 times, and then stirred at 15° C. for 2 hr under H₂ atmosphere. LC-MS showed Reactant 1 was consumed completely and one main peak with desired m/z or desired mass was detected. The mixture was filtered and washed with THF:TFE=3:1. The filtrate was concentrated under reduce pressure. Compound 7 (8 g, crude) was obtained as a white solid.

LCMS: RT=0.890 min, MS cal.: 410.2, [M+H]⁺=411.2

¹H NMR (400 MHz, DMSO-d6) δ=11.53-11.44 (m, 1H), 7.82 (d, J=1.2 Hz, 1H), 7.32 (br dd, J=7.2, 8.4 Hz, 1H), 7.26 (d, J=1.2 Hz, 1H), 6.12 (br d, J=8.8 Hz, 1H), 5.97 (br d, J=7.2 Hz, 1H), 3.96 (s, 3H), 3.88 (d, J=5.2 Hz, 7H), 3.79 (s, 2H), 2.90 (br d, J=11.6 Hz, 2H), 2.40 (br s, 1H), 2.11 (br t, J=10.8 Hz, 2H), 1.78 (br d, J=12.0 Hz, 2H), 1.66-1.51 (m, 2H)

### General Procedure for Preparation of Intermediate 8:

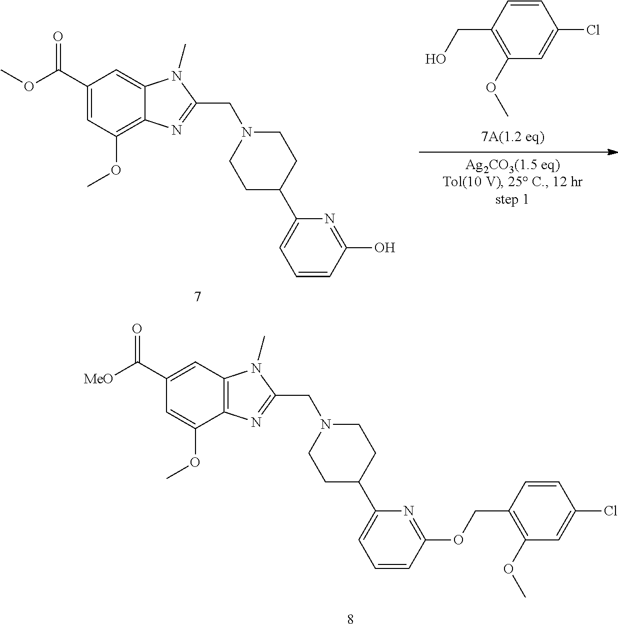

To a solution of 7 (100 mg, 243.63 μmol, 1 eq) and 7A (86.06 mg, 365.44 μmol, 1.5 eq) in Toluene (2 mL) was added Ag₂CO₃ (134.36 mg, 487.25 μmol, 2 eq). The mixture was stirred at 90° C. for 12 hr. LCMS showed 7 was consumed completely and one main peak with desired mass was detected. The mixture was filtered and washed with DCM. Then the filtrate was concentrated under reduce pressure. The residue was poured into ice-water (2 mL) and stirred for 5 min. The aqueous phase was extracted with DCM (5 mL*3). The combined organic phase was washed with brine (5 mL), dried with anhydrous Na₂SO₄, filtered and concentrated in vacuum. The residue was purified by column chromatography (SiO₂, Petroleum ether/Ethyl acetate=20/1 to 1/1). 8 (80 mg, 141.58 μmol, 58.11% yield) was obtained as a yellow solid.

LCMS: product Rt=0.487 min;

LCMS: RT=0.487 min, MS cal.: 564.2, [M+H]⁺=565.3

¹H NMR (400 MHz, CHCl3-d) δ=7.79 (s, 1H), 7.55-7.46 (m, 1H), 7.44-7.35 (m, 2H), 6.92 (br d, J=8.0 Hz, 1H), 6.88 (d, J=1.6 Hz, 1H), 6.76-6.69 (m, 1H), 6.65-6.58 (m, 1H), 5.38-5.31 (m, 2H), 4.07 (s, 3H), 3.97 (s, 3H), 3.91-3.82 (m, 6H), 3.01-2.92 (m, 1H), 2.67-2.57 (m, 1H), 2.34-2.26 (m, 1H), 1.98-1.85 (m, 2H), 1.57 (br d, J=1.6 Hz, 5H)

General Procedure for Preparation of Compound 348 (7 below)-7:

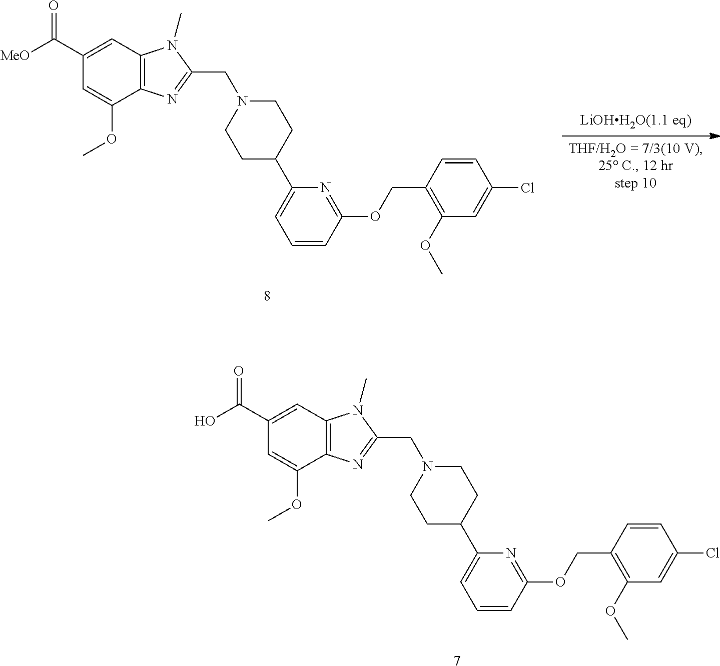

A mixture of 8 (70 mg, 123.88 μmol, 1 eq), LiOH·H₂O (10.40 mg, 247.76 μmol, 2 eq) in THF (2.1 mL) and H₂O (0.3 mL) was stirred at 25° C. for 12 hr. LCMS showed 8 was consumed completely and one main peak with desired mass was detected. The mixture was concentrated under reduce pressure to remove solvent. The residue was diluted with CH₃CN 0.5 mL and filtered. The crude product was purified by prep-HPLC (column: Waters Xbridge BEH C18 100*30 mm*10 um; mobile phase: [H₂O (10 mM NH₄HCO₃)-ACN]; gradient: 30%-60% B over 8.0 min). 2-((4-(6-((4-Chloro-2-methoxybenzyl)oxy)pyridin-2-yl)piperidin-1-yl)methyl)-4-methoxy-1-methyl-1H-benzo[d]imidazole-6-carboxylic acid (28.48 mg, 51.68 μmol, 41.72% yield, 100% purity) was obtained as a white solid.

LCMS: RT=1.150 min, MS cal.: 550.2, [M+H]⁺=551.1

LCMS: RT=2.323 min, MS cal.: 550.2, [M+H]⁺=551.3

HPLC: RT=11.291 min, purity: 98.68%

¹H NMR (400 MHz, DMSO-d6) δ=7.77 (s, 1H), 7.60 (t, J=8.0 Hz, 1H), 7.39 (d, J=8.0 Hz, 1H), 7.26 (s, 1H), 7.11 (d, J=1.6 Hz, 1H), 6.98 (dd, J=2.0, 8.0 Hz, 1H), 6.84 (d, J=7.2 Hz, 1H), 6.65 (d, J=8.0 Hz, 1H), 5.27 (s, 2H), 3.94 (s, 3H), 3.89 (s, 3H), 3.83 (s, 3H), 3.79 (s, 2H), 2.93 (br d, J=12 Hz, 2H), 2.63-2.55 (m, 1H), 2.18 (br t, J=10.4 Hz, 2H), 1.82-1.66 (m, 4H)

### Example A87—Preparation of Compound 349. 2-((4-(6-((4-Cyano-2-fluorobenzyl)oxy)pyridin-2-yl)piperidin-1-yl)methyl)-4-(methoxy-d₃)-1-methyl-1H-benzo[d]imidazole-6-carboxylic acid (Compound 349) (3)

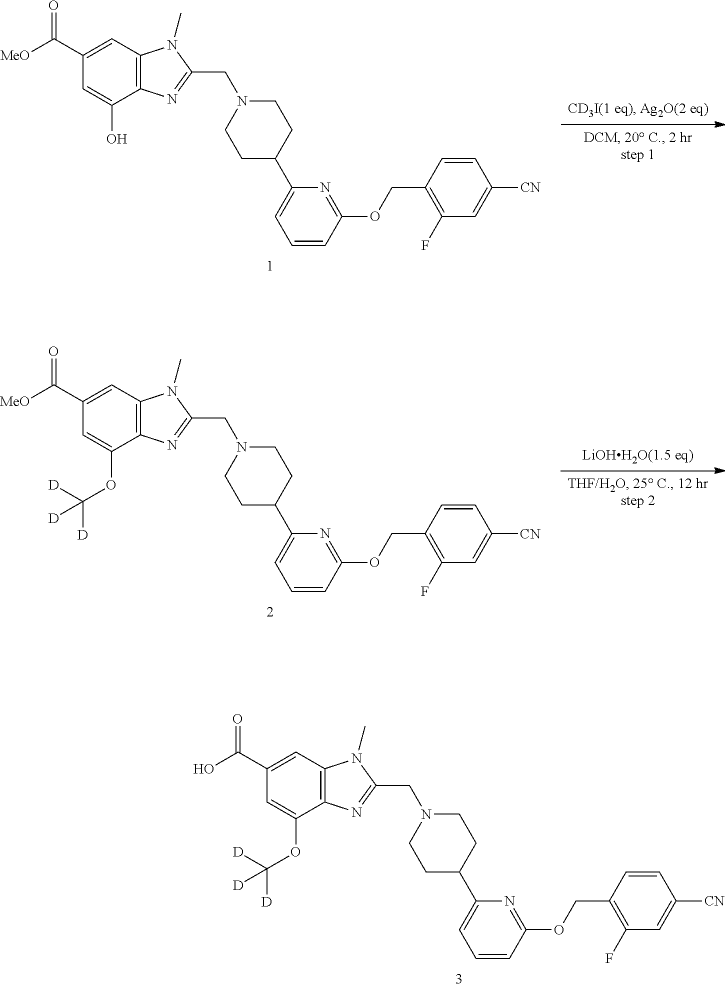

### General Procedure for Preparation of Intermediate 2:

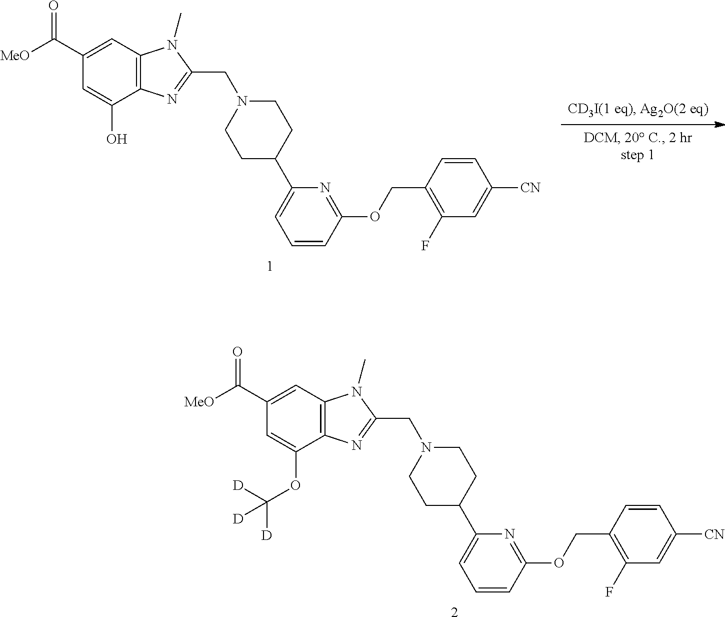

DMF (1 mL) was charged to the three-necked round bottom flask, then 1 (100 mg, 188.84 μmol, 1 eq) and Ag₂O (87.52 mg, 377.67 μmol, 2 eq) was added to the mixture at 20° C. CD₃I (27.37 mg, 188.84 μmol, 11.75 μL, 1 eq) was added dropwise to the reaction mixture at 20° C. After the addition, the mixture was stirred at 20° C. for 2 hr. LC-MS (RT=1.483 min) showed 1 was consumed completely and one main peak with desired mass was detected. The reaction mixture was added to H₂O 6 mL at 25° C. The mixture was extracted by EtOAc (6 mL*3). Then organic phase was combined and washed by H₂O (6 mL), dried with Na₂SO₄, filtered. The organic phase was concentrated under reduced pressure at 35° C. to give a residue. The residue was purified by column chromatography (SiO₂, Petroleum ether/Ethyl acetate=10/1 to Ethyl acetate/Methanol=0/1). 2 (70 mg, 128.06 μmol, 67% yield) was obtained as a yellow solid.

LCMS: RT=1.495 min, MS cal.: 546.2, [M+H]⁺=547.2

¹H NMR (400 MHz, DMSO-d₆) δ=7.88 (d, J=10.0 Hz, 1H), 7.83 (d, J=1.2 Hz, 1H), 7.71-7.67 (m, 2H), 7.64 (t, J=7.6 Hz, 1H), 7.26 (d, J=1.2 Hz, 1H), 6.87 (d, J=7.2 Hz, 1H), 6.71 (d, J=8.4 Hz, 1H), 5.45 (s, 2H), 3.92 (s, 3H), 3.88 (s, 3H), 3.80 (s, 2H), 2.90 (d, J=12.8 Hz, 2H), 2.57 (s, 1H), 2.25-2.11 (m, 2H), 1.78-1.58 (m, 4H)

### General Procedure for Preparation of Compound 349 (3):

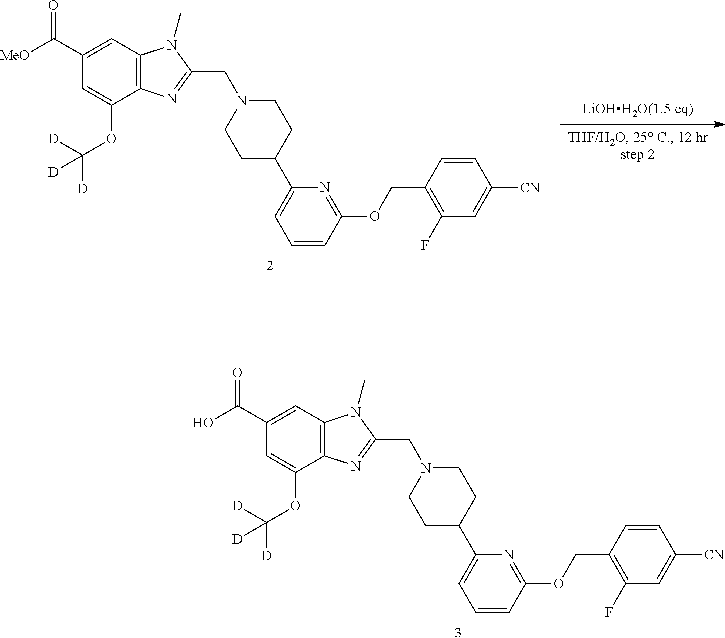

To a solution of 2 (60 mg, 109.77 μmol, 1 eq) in THF (0.7 mL) was added LiOH·H₂O (6.91 mg, 164.65 μmol, 1.5 eq) in H₂O (0.3 mL). The mixture was stirred at 25° C. for 12 hr. LC-MS (RT=1.076 min) showed 2 was consumed completely and one main peak with desired mass was detected. The reaction mixture was filtered. The filtrate was purified by prep-HPLC (column: Waters Xbridge Prep OBD C₁₈ 150*40 mm*10 um; mobile phase: [H₂O (10 mM NH₄HCO₃)-ACN]; gradient: 20%-60% B over 8.0 min). The residue was repurified by prep-HPLC (column: Waters Xbridge BEH C₁₈ 100*30 mm*10 um; mobile phase: [H₂O (10 mM NH₄HCO₃)-ACN]; gradient: 25%-55% B over 8.0 min). 2-((4-(6-((4-Cyano-2-fluorobenzyl)oxy)pyridin-2-yl)piperidin-1-yl)methyl)-4-(methoxy-d₃)-1-methyl-1H-benzo[d]imidazole-6-carboxylic acid (26.06 mg, 48.93 μmol, 44% yield) was obtained as a white solid.

LCMS: RT=2.536 min, MS cal.: 532.2, [M+H]⁺=533.3

HPLC: RT=9.787 min, purity: 98.81%

¹H NMR (400 MHz, MeOH-d4) δ=7.85 (d, J=1.2 Hz, 1H), 7.67 (t, J=7.6 Hz, 1H), 7.62-7.51 (m, 3H), 7.45 (d, J=1.2 Hz, 1H), 6.83 (d, J=7.2 Hz, 1H), 6.67 (d, J=8.0 Hz, 1H), 5.51 (s, 2H), 3.99 (s, 3H), 3.89 (s, 2H), 3.02 (d, J=11.2 Hz, 2H), 2.68-2.57 (m, 1H), 2.36-2.27 (m, 2H), 1.87-1.78 (m, 4H)

### Example A88—Preparation of Compound 350. 2-((4-(6-((6-Cyano-4-fluoropyridin-3-yl)methoxy)pyridin-2-yl)piperidin-1-yl)methyl)-4-(difluoromethoxy)-1-methyl-1H-benzo[d]imidazole-6-carboxylic acid (Compound 350) (5)

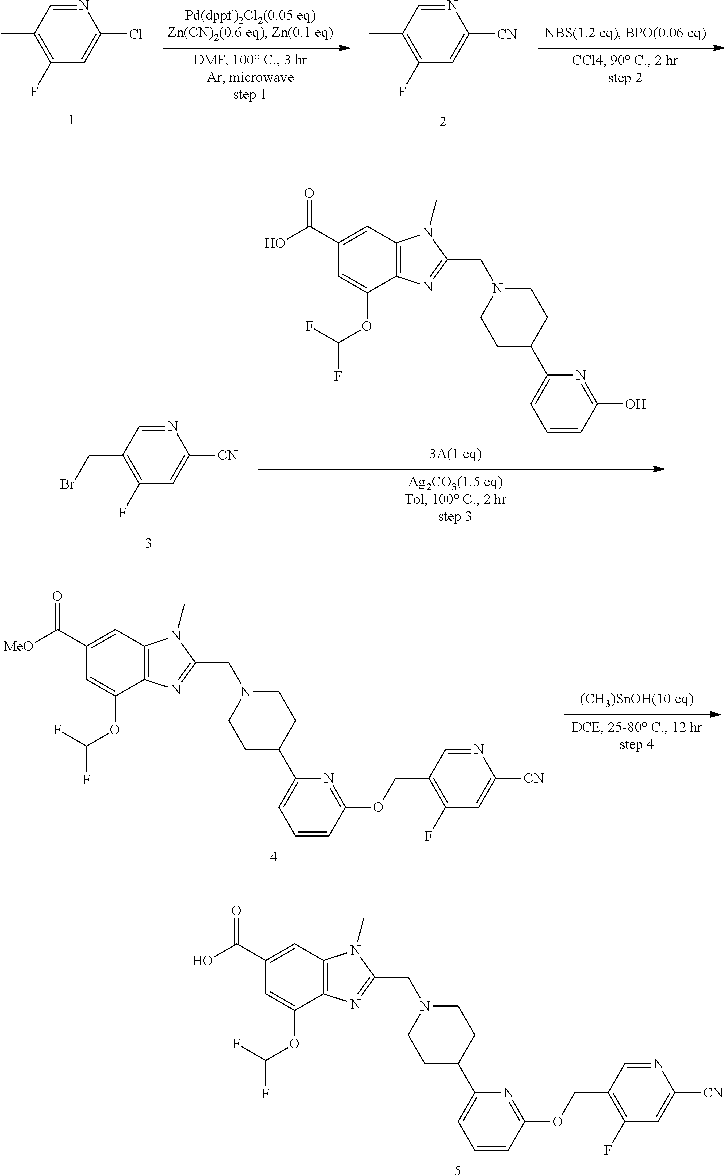

### General Procedure for Preparation of Intermediate 2:

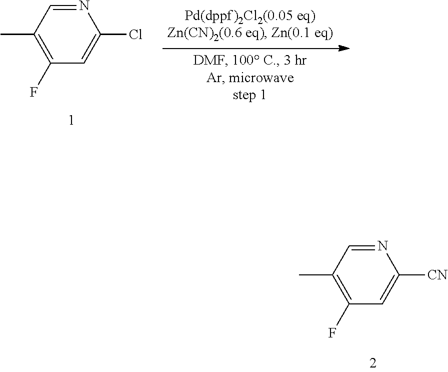

DMF (5 mL) was charged to the microwave tube, the 1 (500 mg, 3.43 mmol, 1 eq) was added to the mixture at 20° C. At 20° C., Zn (CN)₂ (242.01 mg, 2.06 mmol, 130.82 μL, 0.6 eq), Zn (22.46 mg, 343.50 μmol, 0.1 eq) and Pd(dppf)Cl₂·CH₂Cl₂ (140.26 mg, 171.75 μmol, 0.05 eq) was added under N₂. The resulting mixture was sparged with argon for 5 minutes then heated to 100° C. in a sealed 20 mL MW vial for 3 hr. TLC (Petroleum ether:Ethyl acetate=3:1, Rf=0.38) indicated 1 was consumed completely and one new spot formed. The suspension was filtered through a pad of Celite, and filter cake was washed with EtOAc 15 mL (5 mL*3). The combined filtrates were diluted with H₂O 30 mL and extracted with EtOAc 60 mL (20 mL*3). The combined organic layers were washed with brine 15 mL, dried over Na₂SO₄, filtered, and concentrated under reduced pressure to give a residue. The residue was purified by column chromatography (SiO₂, Petroleum ether/Ethyl acetate=10/1 to 0/1). 2 (320 mg, 2.35 mmol, 68.44% yield) was obtained as colorless oil.

LCMS: RT=1.022 min, MS cal.: 136.0, [M+H]⁺=137.1

¹H NMR (400 MHz, CHCl3-d) δ=8.55 (d, J=9.6 Hz, 1H), 7.40 (d, J=8.4 Hz, 1H), 2.38 (s, 3H)

### General Procedure for Preparation of Intermediate 3:

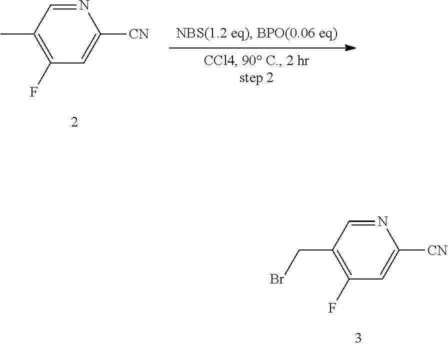

CCl₄ (1.5 mL) was charged to the three-necked round bottom flask, then 2 (150 mg, 1.10 mmol, 1 eq) and NBS (235.35 mg, 1.32 mmol, 1.2 eq) was added. At 0° C., BPO (16.01 mg, 66.12 μmol, 0.06 eq) was added to the reaction mixture. After the addition, the mixture was stirred at 90° C. for 2 hr. LC-MS (RT=1.280 min) showed 2 was consumed completely and desired mass was detected. The reaction mixture was quenched by addition saturated Na₂S₂O₃ solution 1 mL at 0° C., and then diluted with H₂O 5 mL and extracted with DCM 5 mL. The organic layer was washed with brine, dried over Na₂SO₄, filtered and concentrated under reduced pressure to give a residue. The residue was purified by column chromatography (SiO₂, Petroleum ether/Ethyl acetate=10/1 to 1/1). 3 (140 mg, 520.88 μmol, 47.27% yield, 80% purity) was obtained as a yellow solid.

LCMS: RT=1.297 min, MS cal.: 215.03, [M+H]⁺=215.0

### General Procedure for Preparation of Intermediate 4:

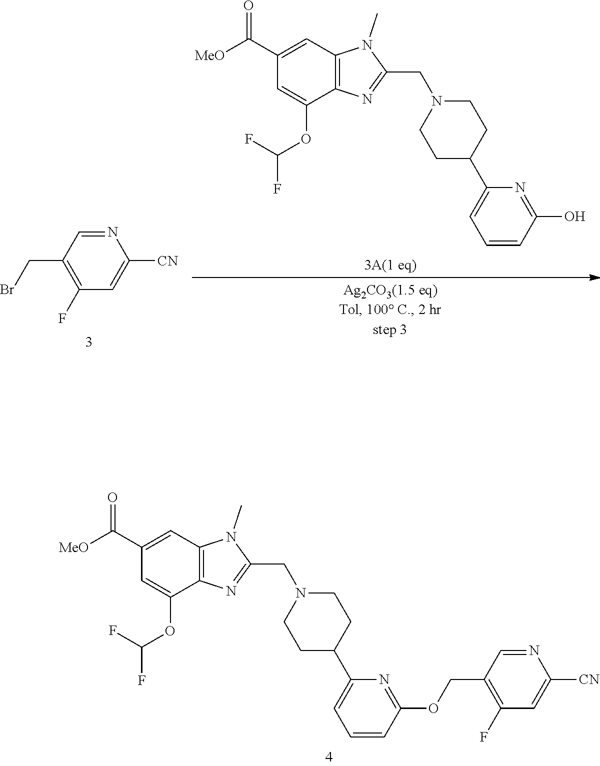 [SMILES: N#Cc1cc(F)c(CBr)cn1 ; COC(=O)c1cc(OC(F)F)c2nc(CN3CCC(c4cccc(O)n4)CC3)n(C)c2c1 ; COC(=O)c1cc(OC(F)F)c2nc(CN3CCC(c4cccc(OCc5cnc(C#N)cc5F)n4)CC3)n(C)c2c1]

A mixture of 3 (130 mg, 483.67 μmol, 1 eq), 3A (215.93 mg, 483.67 μmol, 1 eq), Ag₂CO₃ (200.05 mg, 725.51 μmol, 32.92 μL, 1.5 eq) in Toluene (2 mL) was degassed and purged with N₂ for 3 times, and then the mixture was stirred at 100° C. for 2 hr under N₂ atmosphere. TLC (Petroleum ether:Ethyl acetate=0:1, Rf=0.50) indicated 3 was consumed completely and one new spot formed. The reaction was clean according to TLC. The reaction mixture was diluted with H₂O 10 mL and extracted with EtOAc 10 mL. The organic layer was washed with brine 10 mL, dried over Na₂SO₄, filtered and concentrated under reduced pressure to give a residue. The residue was purified by column chromatography (SiO₂, Petroleum ether/Ethyl acetate=5/1 to 0/1). 4 (150 mg, 258.37 μmol, 53.42% yield) was obtained as a yellow solid.

LCMS: RT=0.454 min, MS cal.: 580.57, [M+H]⁺=581.2

¹H NMR (400 MHz, CHCl3-d) δ=8.86 (d, J=9.2 Hz, 1H), 7.99 (d, J=1.2 Hz, 1H), 7.72 (s, 1H), 7.56-7.52 (m, 1H), 7.45 (d, J=8.8 Hz, 1H), 7.32 (t, J=74.4 Hz, 1H), 6.78 (d, J=7.2 Hz, 1H), 6.65 (d, J=8.0 Hz, 1H), 5.54 (s, 2H), 4.01 (s, 3H), 3.97 (s, 3H), 3.90 (br s, 2H), 2.97 (br d, J=9.6 Hz, 2H), 2.66-2.58 (m, 1H), 2.31 (br s, 2H), 1.88-1.75 (m, 4H)

### General Procedure for Preparation of Compound 350 (5):

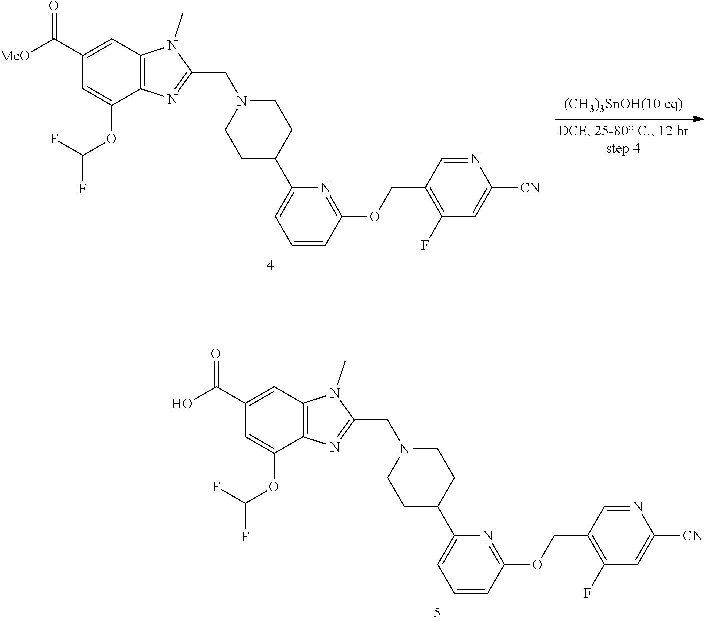 [SMILES: COC(=O)c1cc(OC(F)F)c2nc(CN3CCC(c4cccc(OCc5cnc(C#N)cc5F)n4)CC3)n(C)c2c1 ; Cn1c(CN2CCC(c3cccc(OCc4cnc(C#N)cc4F)n3)CC2)nc2c(OC(F)F)cc(C(=O)O)cc21]

DCE (1.3 mL) was charged to the three-necked round bottom flask, then 4 (130 mg, 223.92 μmol, 1 eq) and (CH₃)₃SnOH (404.90 mg, 2.24 mmol, 10 eq) was added to the reaction mixture at 25° C. After the addition, the mixture was stirred at 80° C. for 12 hr. LC-MS (RT=1.070 min) showed 4 was consumed completely and desired mass was detected. The reaction mixture was added saturated KF solution (3 ml) at 25° C., the mixture was diluted with H₂O 5 mL and extracted with DCM 20 mL (5 mL*4). The combined organic layers were dried over Na₂SO₄, filtered, and concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 30%-60% B over 8.0 min) for 1st round. The residue was purified by prep-HPLC (column: Phenomenex Luna C18 100*30 mm*3 um; mobile phase: [H₂O (0.2% FA)-ACN]; gradient: 10%-50% B over 8.0 min) for 2nd round. 2-((4-(6-((6-Cyano-4-fluoropyridin-3-yl)methoxy)pyridin-2-yl)piperidin-1-yl)methyl)-4-(difluoromethoxy)-1-methyl-1H-benzo[d]imidazole-6-carboxylic acid (4.36 mg, 7.11 μmol, 3.18% yield, 92.39% purity) was obtained as a white solid.

LCMS: RT=2.229 min, MS cal.: 566.54, [M+H]⁺=567.2

HPLC: RT=10.025 min, purity: 92.39%

¹H NMR (400 MHz, DMSO-d₆) δ=8.90 (d, J=9.6 Hz, 1H), 8.21 (d, J=9.6 Hz, 1H), 8.08 (s, 1H), 7.66-7.62 (m, 1H), 7.63 (t, J=74.4 Hz, 1H), 7.52 (s, 1H), 6.89 (d, J=7.6 Hz, 1H), 6.71 (d, J=8.0 Hz, 1H), 5.49 (s, 2H), 3.98 (s, 3H), 3.86 (s, 2H), 2.93 (br d, J=11.6 Hz, 2H), 2.58 (br d, J=3.6 Hz, 1H), 2.21 (br t, J=10.8 Hz, 2H), 1.77-1.65 (m, 4H)

### Example A89—Preparation of Compound 351. 2-((4-(6-((4-Chloro-2-fluorobenzyl)oxy)pyridin-2-yl)piperidin-1-yl)methyl)-4-(1-fluoropropoxy)-1-methyl-1H-benzo[d]imidazole-6-carboxylic acid (Compound 351) (6)

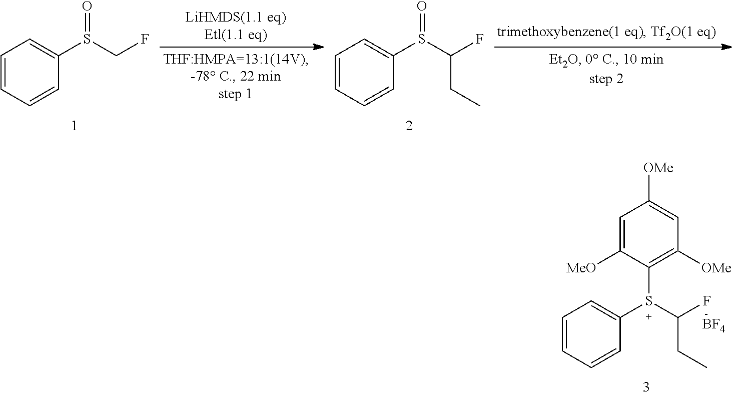

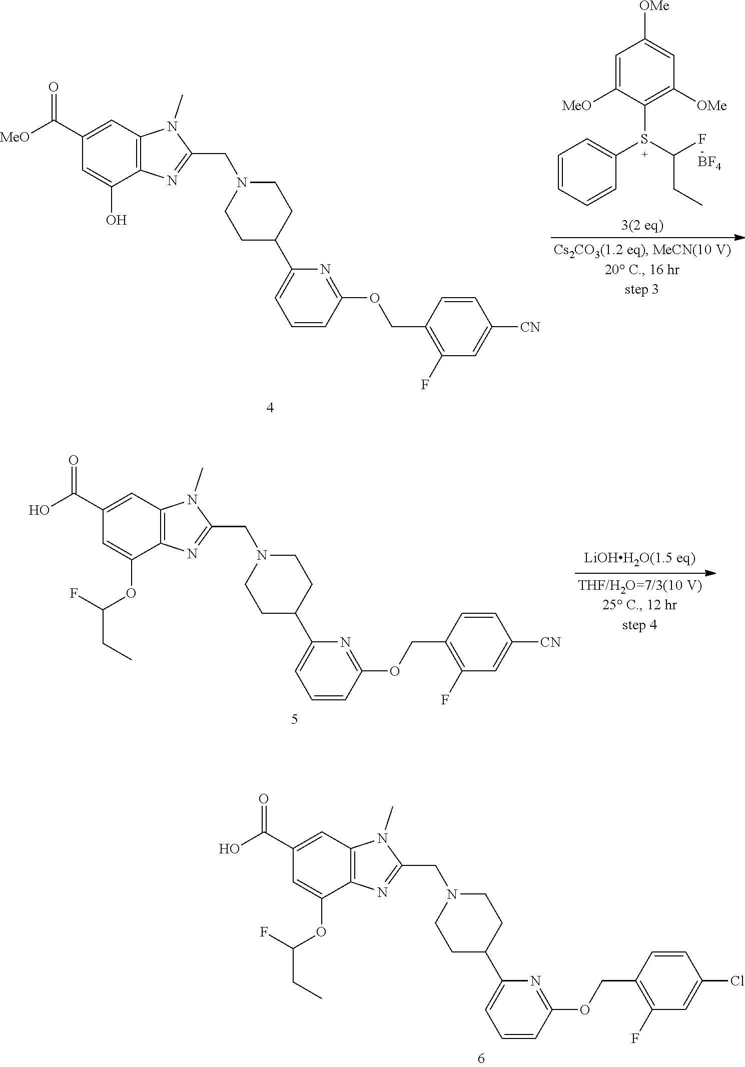

### General Procedure for Preparation of Intermediate 2:

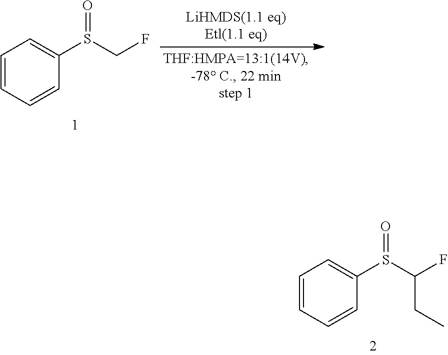

Equip a 100 mL three-necked round bottom flask, addition funnel and thermometer, N₂ balloon etc. THF (13 mL) and HMPA (1 mL) was charged to the 100 mL three-necked round bottom flask, then 1 (1 g, 6.32 mmol, 1 eq) and EtI (1.08 g, 6.95 mmol, 556.16 μL, 1.1 eq) was added within 1 min. At −78° C. inner temperature, LiHMDS (1 M, 6.95 mL, 1.1 eq) was added to the reaction mixture at −78° C. within 1 min. After the addition, the mixture was stirred at −78° C. for 20 min. TLC (Petroleum ether/Ethyl acetate=5/1, R_{f}=0.67, 0.8) indicated 1 was consumed completely and two new spot formed. The reaction was clean according to TLC. Then saturated aqueous NH₄Cl solution (10 mL) and water (20 mL) were added. The mixture was warmed to RT and MTBE (30 mL) was added. The layers were separated, and the aqueous layer was extracted with MTBE (20 mL*3). The combined organic layers were washed with water (20 mL*1), dried over MgSO₄ and concentrated under vacuum. The residue was purified by column chromatography (SiO₂, Petroleum ether/Ethyl acetate=100/1 to 0/1). 2 (320 mg, 1.55 mmol, 24% yield, 90% purity) was obtained as a white solid.

### General Procedure for Preparation of Intermediate 3:

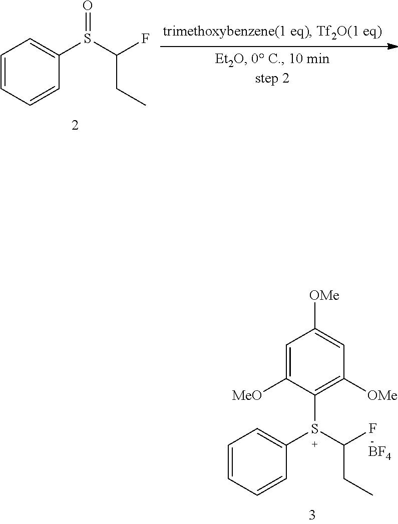

Equip a 50 mL round bottom flask. Et₂O (15 mL) was charged to the 50 mL three necked round bottom flask, then 2 (120 mg, 644.31 μmol, 1 eq) and 1,3,5-trimethoxybenzene (108.37 mg, 644.31 μmol, 1 eq) was added to the mixture at 25° C. The resulting solution was cooled down to 0° C. and Tf₂O (181.78 mg, 644.31 μmol, 106.31 μL, 1 eq) was added dropwise. After the addition, the reaction was stirred for 10 mins. LCMS showed 2 was consumed completely and one main peak with desired mass was detected. After 10 min, then Et₂O was removed with a syringe. Then, Et₂O (5 mL*2) was added and removed with a syringe. This operation was repeated three. DCM (5 mL) was then added, and the solution was washed with aqueous 1M NaBF₄ solution (3×8 mL). The organic layer was dried over Na₂SO₄ and concentrated under vacuum. 3 (250 mg, crude, BF4-) was obtained as a gray solid.

### General Procedure for Preparation of Intermediate 5:

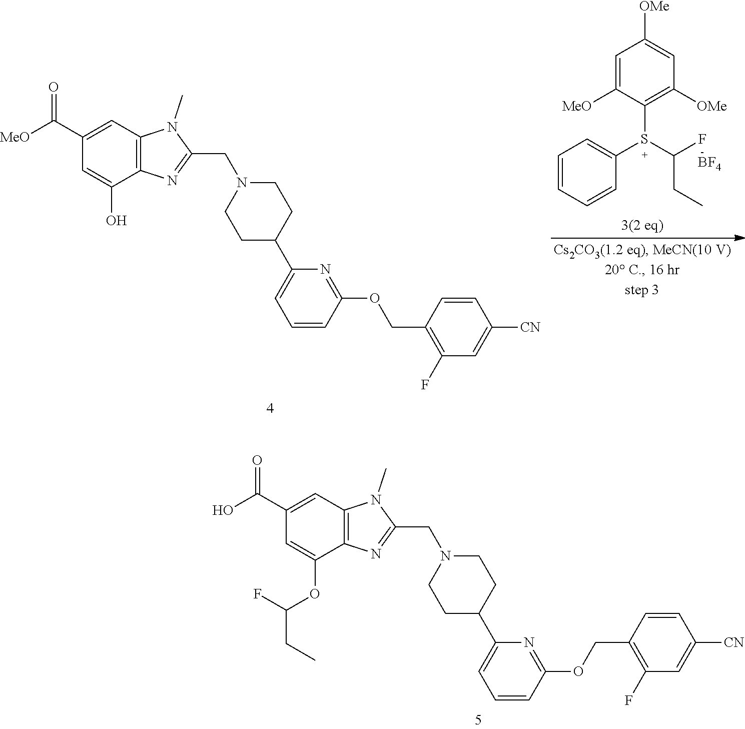

A mixture of 4 (100 mg, 185.53 μmol, 1 eq), 3 (157.42 mg, 371.06 μmol, 2 eq, BF4-), Cs₂CO₃ (72.54 mg, 222.64 μmol, 1.2 eq), in MeCN (1 mL) was degassed and purged with N₂ for 3 times, and then the mixture was stirred at 20° C. for 16 hr under N₂ atmosphere. LCMS showed 4 was consumed completely and one main peak with desired mass was detected. The reaction mixture was diluted with H₂O 3 mL and extracted with EtOAc 6 mL (2 mL*3), dried over Na₂SO₄, filtered, and concentrated under reduced pressure to give a residue. The residue was purified by prep-TLC (SiO₂, Petroleum ether/Ethyl acetate=2:1). 5 (55 mg, 88.14 μmol, 47% yield, 96% purity) was obtained as a white solid.

LCMS: RT=0.530 min, MS cal.: 598.2, 599.2, [M+H]⁺=599.2, 600.2

### General Procedure for Preparation of Compound 351 (6):

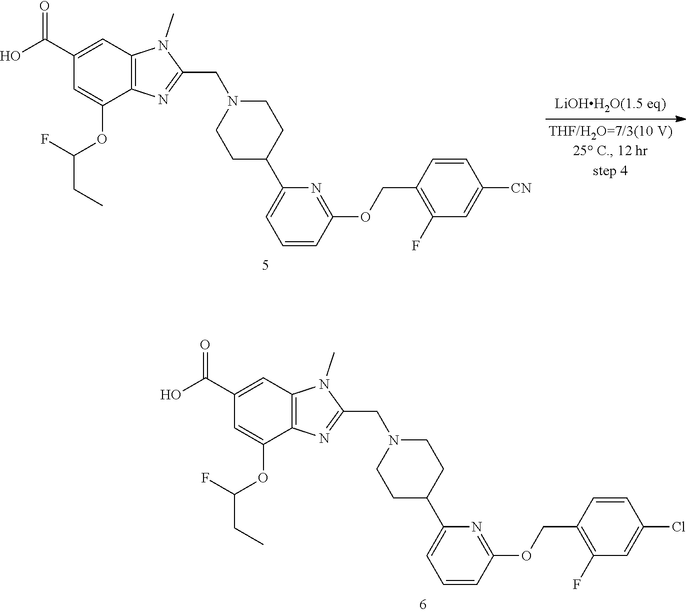 [SMILES: CCC(F)Oc1cc(C(=O)O)cc2c1nc(CN1CCC(c3cccc(OCc4ccc(C#N)cc4F)n3)CC1)n2C ; CCC(F)Oc1cc(C(=O)O)cc2c1nc(CN1CCC(c3cccc(OCc4ccc(Cl)cc4F)n3)CC1)n2C]

A mixture of 5 (60 mg, 100.16 μmol, 1 eq), LiOH·H₂O (6.30 mg, 150.23 μmol, 1.5 eq) in THF (0.42 mL) and H₂O (0.18 mL) was degassed and purged with N₂ for 3 times, and then the mixture was stirred at 25° C. for 24 hr under N₂ atmosphere. LC-MS showed Compound 5 was consumed completely and one main peak with desired mass was detected. The mixture was concentrated. The residue was purified by prep-HPLC (neutral condition; column: Waters Xbridge Prep OBD C18150*40 mm*10 um; mobile phase: [H₂O (10 mM NH₄HCO₃)-ACN]; gradient: 30%-60% B over 8.0 min). 2-((4-(6-((4-Chloro-2-fluorobenzyl)oxy)pyridin-2-yl)piperidin-1-yl)methyl)-4-(1-fluoropropoxy)-1-methyl-1H-benzo[d]imidazole-6-carboxylic acid (18.89 mg, 31.97 μmol, 31.92% yield, 99.24% purity) was obtained as a pink solid.

LCMS: RT=2.905 min, MS cal.: 584.2, 585.2, [M+H]⁺=585.2, 587.2

HPLC: RT=12.915 purity: 99.24%

¹H NMR (400 MHz, DMSO-d6) δ=7.87 (s, 1H), 7.619 (t, J=8.0 Hz, 1H), 7.55 (t, J=8.0 Hz, 1H), 7.48 (s, 1H), 7.45 (dd, J=10.0, 2.0 Hz, 1H), 7.29 (dd, J=8.0, 2.0 Hz, 1H), 6.86 (d, J=7.2 Hz, 1H), 6.66 (d, J=8.4 Hz, 1H), 6.39 (dt, J=62.4, 4.8 Hz, 1H), 5.35 (s, 2H), 3.92 (s, 3H), 3.82 (d, J=4.4 Hz, 2H), 2.95-2.92 (m, 2H), 2.59-2.51 (m, 1H), 2.20-2.15 (m, 2H), 1.99-1.95 (m, 2H), 1.80-1.68 (m, 4H), 1.06 (t, J=7.6 Hz, 3H)

### Example A90—Preparation of Compound 352. 2-((4-(6-((4-Cyano-2-fluorobenzyl)oxy)pyridin-2-yl)piperidin-1-yl)methyl)-4-(4-fluorophenoxy)-1-methyl-1H-benzo[d]imidazole-6-carboxylic acid (Compound 352) (7)

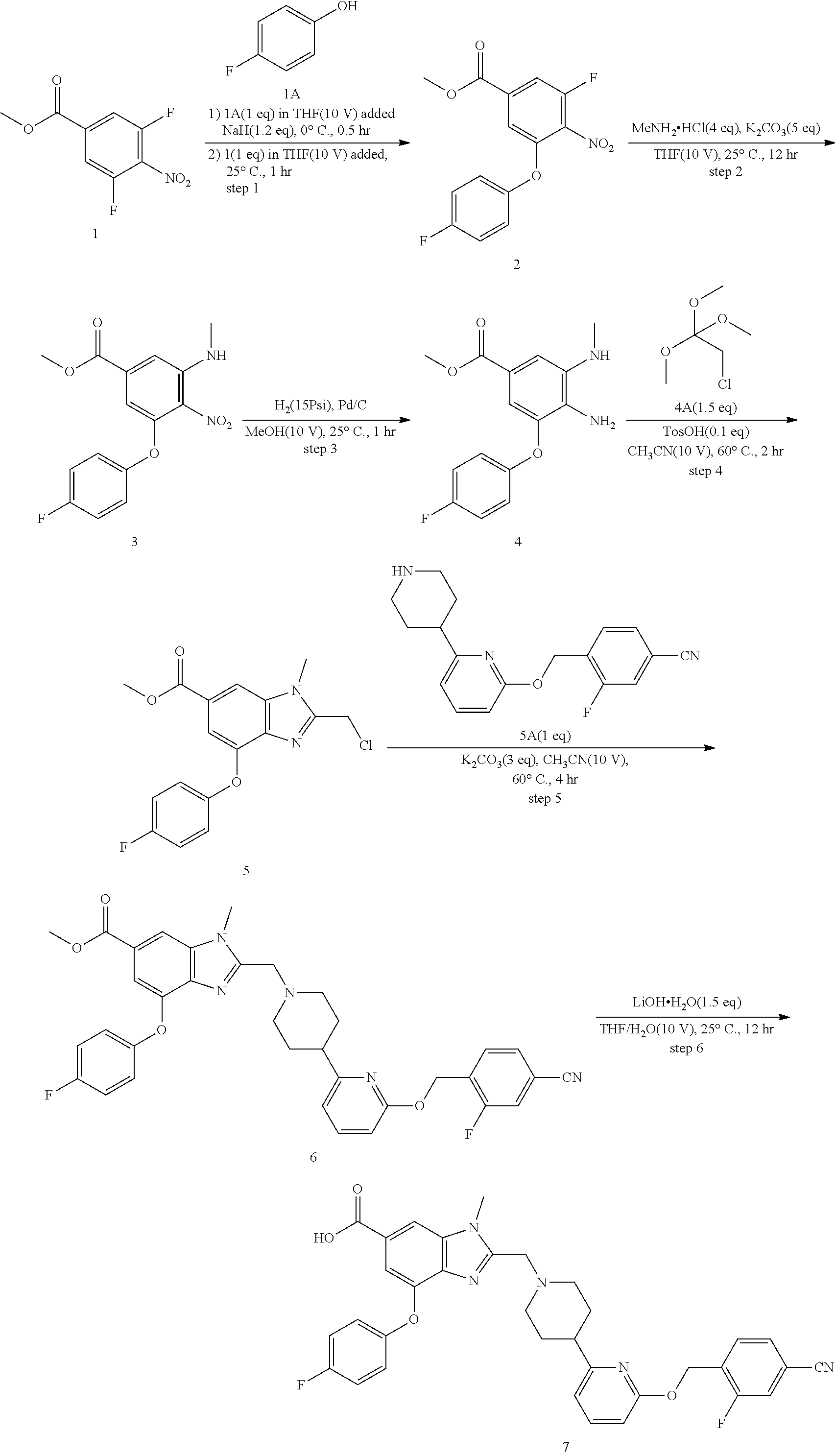

### General Procedure for Preparation of Intermediate 2:

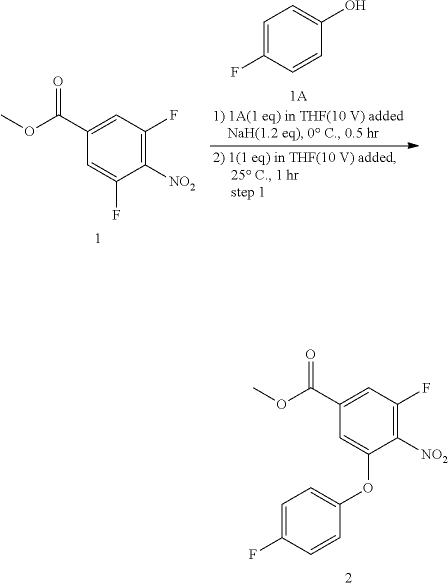 [SMILES: Oc1ccc(F)cc1 ; COC(=O)c1cc(F)c([N+](=O)[O-])c(F)c1 ; COC(=O)c1cc(F)c([N+](=O)[O-])c(Oc2ccc(F)cc2)c1]

THF (5 mL) was charged to the round bottom flask, then 1A (516.30 mg, 4.61 mmol, 1 eq) was added to the mixture at 25° C. At 0° C., NaH (221.05 mg, 5.53 mmol, 60% purity, 1.2 eq) was added in portions at 0° C. The mixture was stirred at 0° C. for 0.5 hr. 1 (1 g, 4.61 mmol, 1 eq) in THF (10 mL) was added dropwise to the mixture at 0° C. After the addition, the mixture was stirred at 25° C. for 1 hr. HPLC showed 1 was consumed completely and a new main peak was detected. The reaction mixture was diluted with H₂O (20 mL) and extracted with EtOAc (30 mL). The combined organic layers were washed with brine (20 mL), dried over Na₂SO₄, filtered and concentrated under reduced pressure to give a residue. The residue was purified by column chromatography (SiO₂, Petroleum ether/Ethyl acetate=100/1 to 10/1). 2 (658 mg, 2.13 mmol, 46.20% yield) was obtained as a yellow oil.

¹H NMR (400 MHz, DMSO-d₆) δ=7.83 (dd, J=1.2, 9.6 Hz, 1H), 7.36-7.32 (m, 4H), 7.25-7.20 (m, 1H), 3.83 (s, 3H)

### General Procedure for Preparation of Intermediate 3:

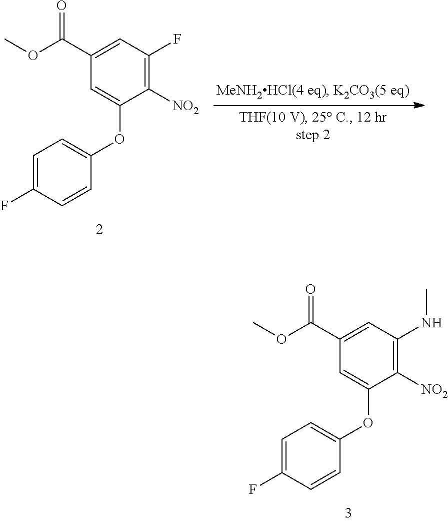 [SMILES: COC(=O)c1cc(F)c([N+](=O)[O-])c(Oc2ccc(F)cc2)c1 ; CNc1cc(C(=O)OC)cc(Oc2ccc(F)cc2)c1[N+](=O)[O-]]

To a solution of 2 (658 mg, 2.13 mmol, 1 eq) in THF (6 mL) was added K₂CO₃ (1.47 g, 10.64 mmol, 5 eq) and MeNH₂·HCl (574.69 mg, 8.51 mmol, 4 eq). The mixture was stirred at 25° C. for 12 hr. LC-MS showed 2 was consumed completely and desired mass was detected. The mixture was diluted with H₂O (20 mL) and extracted with EtOAc (30 mL). The combined organic layers were washed with brine (20 mL), dried over Na₂SO₄, filtered and concentrated under reduced pressure to give a residue. 3 (630 mg, 1.97 mmol, 92.44% yield) was obtained as a red oil.

LCMS: RT=1.364 min, MS cal.: 320.08, [M+H]⁺=321.0

### General Procedure for Preparation of Intermediate 4:

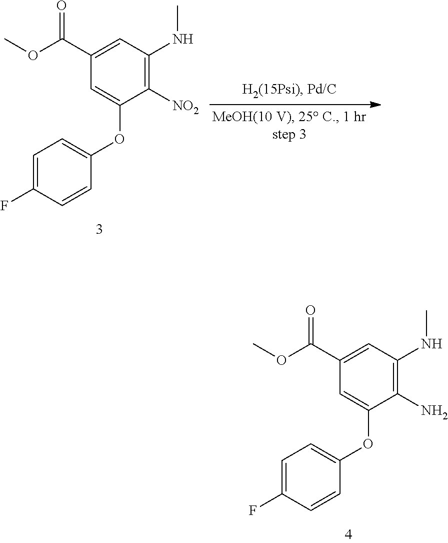

MeOH (5 mL) was charged to the round bottom flask, then 3 (520 mg, 1.62 mmol, 1 eq) was added to the mixture at 25° C. Pd/C (260.00 mg, 10% purity) was added to the reaction mixture at 25° C. After the addition, the mixture was stirred at 25° C. for 1 hr under H₂ atmosphere (15 psi). LCMS showed 2 was consumed completely and desired mass was detected. The reaction mixture was filtered through celite pad. The filter cake was rinsed with MeOH (20 ml*3), and the filtrate was concentrated. 4 (470 mg, 1.62 mmol, 99.72% yield) was obtained as a colorless oil.

LCMS: RT=1.239 min, MS cal.: 290.11, [M+H]⁺=291.0

¹H NMR (400 MHz, DMSO-d₆) δ=7.20-7.13 (m, 2H), 6.98-6.91 (m, 2H), 6.84 (d, J=1.2 Hz, 1H), 5.27-5.18 (m, 2H), 5.15 (br d, J=4.8 Hz, 1H), 3.71 (s, 3H), 2.79 (d, J=5.2 Hz, 3H)

### General Procedure for Preparation of Intermediate 5:

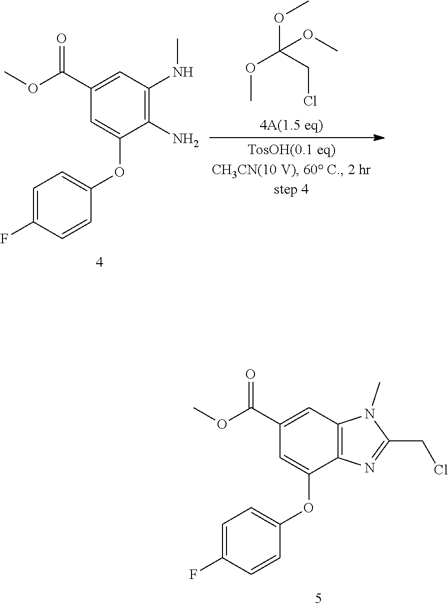

To a solution of 4 (460 mg, 1.58 mmol, 1 eq), 4A (367.46 mg, 2.38 mmol, 320.36 μL, 1.5 eq) in CH₃CN (5 mL) was added TosOH (27.29 mg, 158.46 μmol, 0.1 eq). The mixture was stirred at 60° C. for 2 hr. LC-MS showed 4 was consumed completely and desired mass was detected. The mixture concentrated under reduced pressure to give a residue. 5 (450 mg, 863.80 μmol, 54.51% yield, p-TSA) was obtained as a white solid.

LCMS: RT=1.259 min, MS cal.: 348.07, [M+H]⁺=349.0

### General Procedure for Preparation of Intermediate 6:

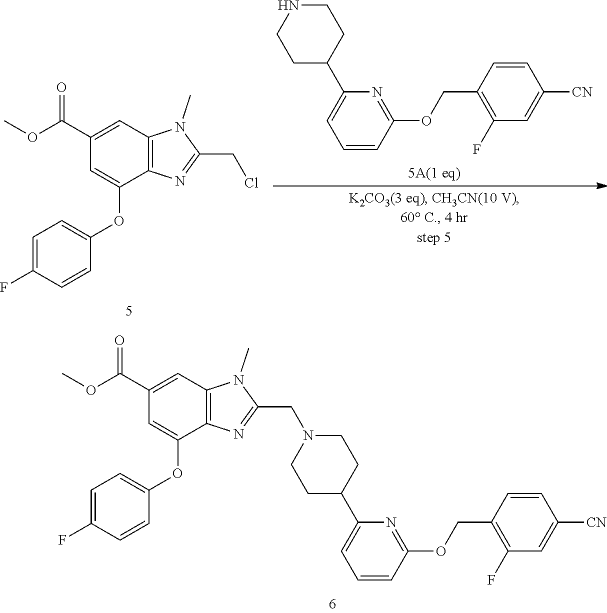 [SMILES: COC(=O)c1cc(Oc2ccc(F)cc2)c2nc(CCl)n(C)c2c1 ; N#Cc1ccc(COc2cccc(C3CCNCC3)n2)c(F)c1 ; COC(=O)c1cc(Oc2ccc(F)cc2)c2nc(CN3CCC(c4cccc(OCc5ccc(C#N)cc5F)n4)CC3)n(C)c2c1]

To a solution of 5 (250 mg, 479.89 μmol, 1 eq, p-TSA), 5A (Synthesized from Int 6) (149.41 mg, 479.89 μmol, 1 eq) in CH₃CN (3 mL) was added K₂CO₃ (198.97 mg, 1.44 mmol, 3 eq). The mixture was stirred at 60° C. for 4 hr. LC-MS showed 5 was consumed completely and desired mass was detected. The mixture was diluted with H₂O (10 mL) and extracted with EtOAc (15 mL). The combined organic layers were washed with brine (10 mL), dried over Na₂SO₄, filtered, and concentrated under reduced pressure to give a residue. The residue was purified by column chromatography (SiO₂, Petroleum ether/Ethyl acetate=3/1 to 0/1). 6 (186 mg, 298.25 μmol, 62.15% yield) was obtained as a white oil.

LCMS: RT=1.636 min, MS cal.: 623.23, [M+H]⁺=624.3

¹H NMR (400 MHz, DMSO-d₆) δ=8.01 (d, J=1.2 Hz, 1H), 7.87 (d, J=10.0 Hz, 1H), 7.69 (d, J=3.6 Hz, 2H), 7.64 (t, J=8.0 Hz, 1H), 7.30-7.20 (m, 2H), 7.19 (d, J=1.2 Hz, 1H), 7.15-7.06 (m, 2H), 6.87 (d, J=7.2 Hz, 1H), 6.71 (d, J=8.0 Hz, 1H), 5.45 (s, 2H), 3.97 (s, 3H), 3.86-3.79 (m, 5H), 2.91 (br d, J=11.2 Hz, 2H), 2.56 (br d, J=4.0 Hz, 1H), 2.18 (br t, J=10.8 Hz, 2H), 1.79-1.59 (m, 4H)

### General Procedure for Preparation of Compound 352 (7):

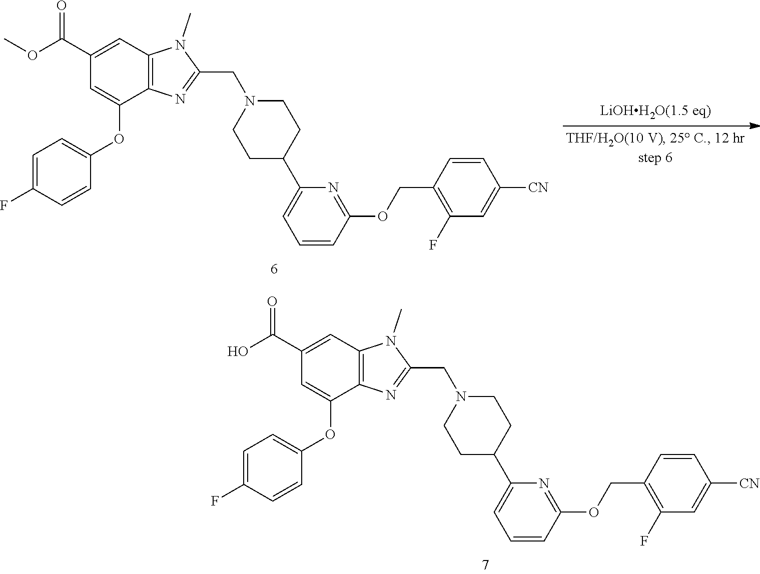 [SMILES: COC(=O)c1cc(Oc2ccc(F)cc2)c2nc(CN3CCC(c4cccc(OCc5ccc(C#N)cc5F)n4)CC3)n(C)c2c1 ; Cn1c(CN2CCC(c3cccc(OCc4ccc(C#N)cc4F)n3)CC2)nc2c(Oc3ccc(F)cc3)cc(C(=O)O)cc21]

To a solution of 6 (250 mg, 400.87 μmol, 1 eq) in THF (1.4 mL) was added LiOH·H₂O (25.23 mg, 601.30 μmol, 1.5 eq) in H₂O (0.6 mL). The mixture was stirred at 25° C. for 12 hr. LC-MS showed 6 was consumed completely and desired mass was detected. The reaction mixture was purified directly. The residue was purified by prep-HPLC neutral condition; column: Waters Xbridge Prep OBD C18 150*40 mm*10 um; mobile phase: [H₂O (10 mM NH₄HCO₃)-ACN]; gradient: 20%-70% B over 8.0 min). 2-((4-(6-((4-Cyano-2-fluorobenzyl)oxy)pyridin-2-yl)piperidin-1-yl)methyl)-4-(4-fluorophenoxy)-1-methyl-1H-benzo[d]imidazole-6-carboxylic acid (28.53 mg, 46.80 μmol, 11.67% yield) was obtained as a white solid.

LCMS: RT=2.775 min, MS cal.: 609.22, [M+H]⁺=610.3

HPLC: RT=11.748 min, purity: 100.0%

¹H NMR (400 MHz, DMSO-d₆) δ=13.27-12.60 (m, 1H), 7.98 (d, J=1.2 Hz, 1H), 7.88 (d, J=10.0 Hz, 1H), 7.69 (d, J=3.6 Hz, 2H), 7.64 (t, J=7.6 Hz, 1H), 7.26-7.20 (m, 2H), 7.17 (d, J=1.2 Hz, 1H), 7.14-7.08 (m, 2H), 6.87 (d, J=7.2 Hz, 1H), 6.71 (d, J=8.0 Hz, 1H), 5.45 (s, 2H), 3.96 (s, 3H), 3.81 (s, 2H), 2.91 (br d, J=11.2 Hz, 2H), 2.62-2.52 (m, 1H), 2.18 (br t, J=10.8 Hz, 2H), 1.80-1.58 (m, 4H)

### Example A91—Preparation of Compound 379. 2-((4-(6-((4-Chloro-2-fluorobenzyl)oxy)pyridin-2-yl)piperidin-1-yl)methyl)-4-((1s,3s)-3-fluorocyclobutoxy)-1-methyl-1H-benzo[d]imidazole-6-carboxylic acid (Compound 379) (3)

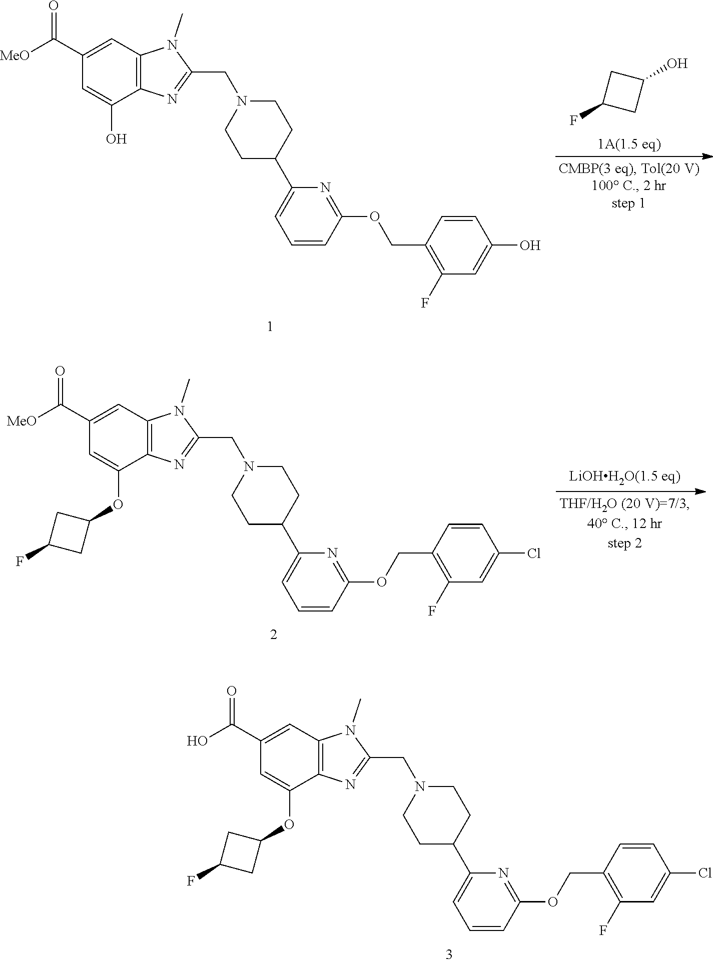 [SMILES: COC(=O)c1cc(O)c2nc(CN3CCC(c4cccc(OCc5ccc(O)cc5F)n4)CC3)n(C)c2c1 ; O[C@H]1C[C@H](F)C1 ; COC(=O)c1cc(O[C@H]2C[C@@H](F)C2)c2nc(CN3CCC(c4cccc(OCc5ccc(Cl)cc5F)n4)CC3)n(C)c2c1 ; Cn1c(CN2CCC(c3cccc(OCc4ccc(Cl)cc4F)n3)CC2)nc2c(O[C@H]3C[C@@H](F)C3)cc(C(=O)O)cc21]

### General Procedure for Preparation of Intermediate 2:

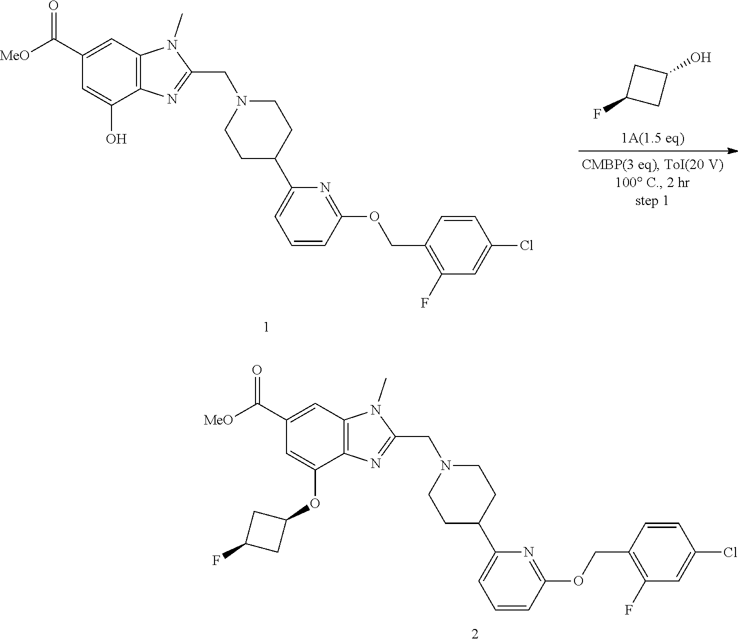 [SMILES: COC(=O)c1cc(O)c2nc(CN3CCC(c4cccc(OCc5ccc(Cl)cc5F)n4)CC3)n(C)c2c1 ; O[C@H]1C[C@H](F)C1 ; COC(=O)c1cc(O[C@H]2C[C@@H](F)C2)c2nc(CN3CCC(c4cccc(OCc5ccc(Cl)cc5F)n4)CC3)n(C)c2c1]

To a solution of 1 (100 mg, 185.53 μmol, 1 eq) (synthesized from Int 3) in Toluene (2 mL) was added CMBP (134.33 mg, 556.59 μmol, 3 eq) and 1A (25.07 mg, 278.29 μmol, 1.5 eq). The mixture was stirred at 100° C. for 2 hr. LC-MS (Rt=0.508 min) showed 1 was consumed completely and one main peak with desired mass was detected. The mixture was diluted with H₂O (30 mL), the aqueous phase was extracted with EtOAc (10 mL*3). The combined organic phase was washed with brine (10 mL), dried with anhydrous Na₂SO₄, filtered and concentrated in vacuum. The residue was purified by column chromatography (SiO₂, PE/EtOAc=0/1 to 0/1). 2 (60 mg, 98.19 μmol, 52.92% yield) was obtained as a brown oil.

LCMS: Rt=0.508 min, MS cal.: 610.22/611.22, [M+H]⁺=611.1/613.1

### General Procedure for Preparation of Compound 379 (3):

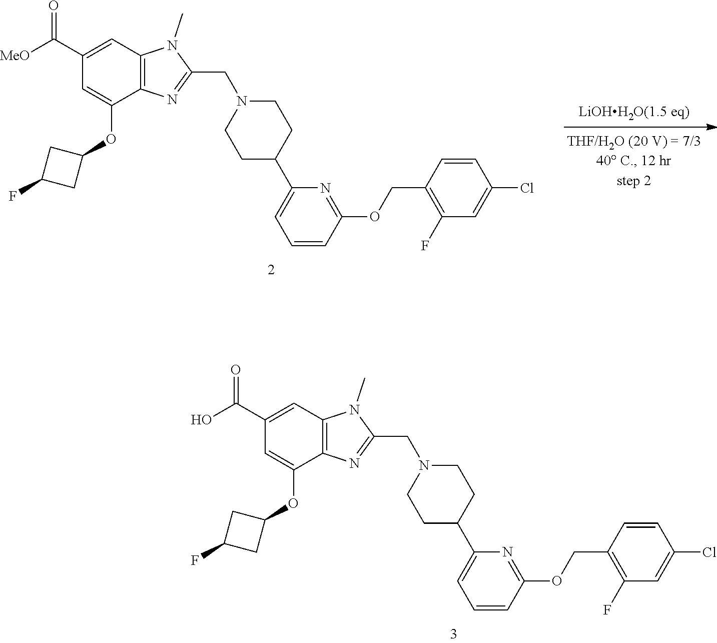 [SMILES: COC(=O)c1cc(O[C@H]2C[C@@H](F)C2)c2nc(CN3CCC(c4cccc(OCc5ccc(Cl)cc5F)n4)CC3)n(C)c2c1 ; Cn1c(CN2CCC(c3cccc(OCc4ccc(Cl)cc4F)n3)CC2)nc2c(O[C@H]3C[C@@H](F)C3)cc(C(=O)O)cc21]

To a solution of 2 (50 mg, 81.82 μmol, 1 eq) in THF (0.7 mL) was added LiOH·H₂O (5.15 mg, 122.73 μmol, 1.5 eq) in H₂O (0.3 mL). The mixture was stirred at 40° C. for 12 hr. LC-MS (Rt=0.473 min) showed 2 was consumed completely and one main peak with desired mass was detected. The reaction was filtered directly. The residue was purified by prep-HPLC (column: Waters Xbridge BEH C18 100*30 mm*10 um; mobile phase: [H₂O (10 mM NH₄HCO₃)-ACN]; gradient: 35%-65% B over 8.0 min). 2-((4-(6-((4-Chloro-2-fluorobenzyl)oxy)pyridin-2-yl)piperidin-1-yl)methyl)-4-((1s,3s)-3-fluorocyclobutoxy)-1-methyl-1H-benzo[d]imidazole-6-carboxylic acid (10.78 mg, 18.06 μmol, 22.07% yield) was obtained as a white solid.

HPLC: Rt=12.236 min, purity 98.71%

LCMS: Rt=2.799 min, MS cal.: 596.2/597.2, [M+H]⁺=597.2/598.2

¹H NMR (400 MHz, DMSO-d₆) δ=7.79 (s, 1H), 7.62 (t, J=8.0 Hz, 1H), 7.55 (t, J=8.4 Hz, 1H), 7.47-7.43 (m, 1H), 7.30-7.26 (m, 1H), 7.13 (s, 1H), 6.87 (d, J=7.2 Hz, 1H), 6.66 (d, J=8.0 Hz, 1H), 5.36 (s, 2H), 5.02 (dt, J=56.4, 6.4 Hz, 1H), 4.68-4.58 (m, 1H), 3.91 (s, 3H), 3.81 (s, 2H), 3.10-3.00 (m, 2H), 2.92 (br d, J=10.4 Hz, 2H), 2.59-2.54 (m, 1H), 2.37-2.26 (m, 2H), 2.19 (br s, 2H), 1.84-1.63 (m, 4H)

### Example A92—Preparation of Compound 353. 2-((4-(6-((4-Chloro-2-fluorobenzyl)oxy)pyridin-2-yl)piperidin-1-yl)methyl)-4-((1r,3r)-3-fluorocyclobutoxy)-1-methyl-1H-benzo[d]imidazole-6-carboxylic acid (Compound 353) (3)

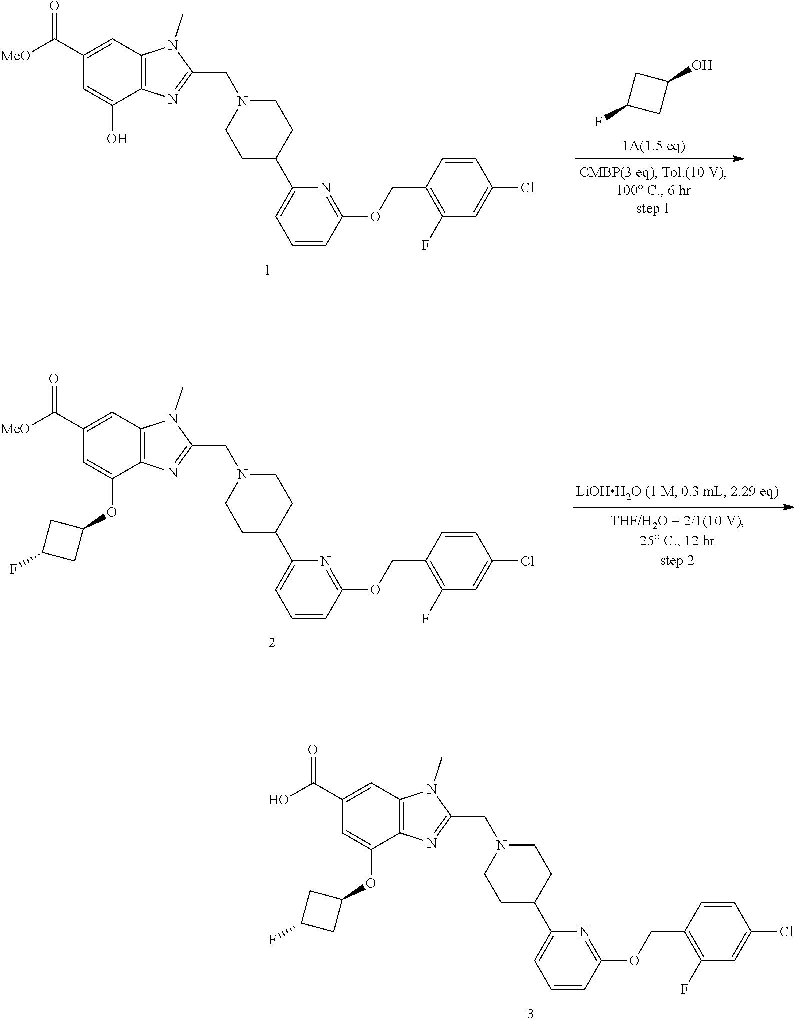 [SMILES: COC(=O)c1cc(O)c2nc(CN3CCC(c4cccc(OCc5ccc(Cl)cc5F)n4)CC3)n(C)c2c1 ; O[C@H]1C[C@@H](F)C1 ; COC(=O)c1cc(O[C@H]2C[C@H](F)C2)c2nc(CN3CCC(c4cccc(OCc5ccc(Cl)cc5F)n4)CC3)n(C)c2c1 ; Cn1c(CN2CCC(c3cccc(OCc4ccc(Cl)cc4F)n3)CC2)nc2c(O[C@H]3C[C@H](F)C3)cc(C(=O)O)cc21]

### General Procedure for Preparation of Intermediate 2:

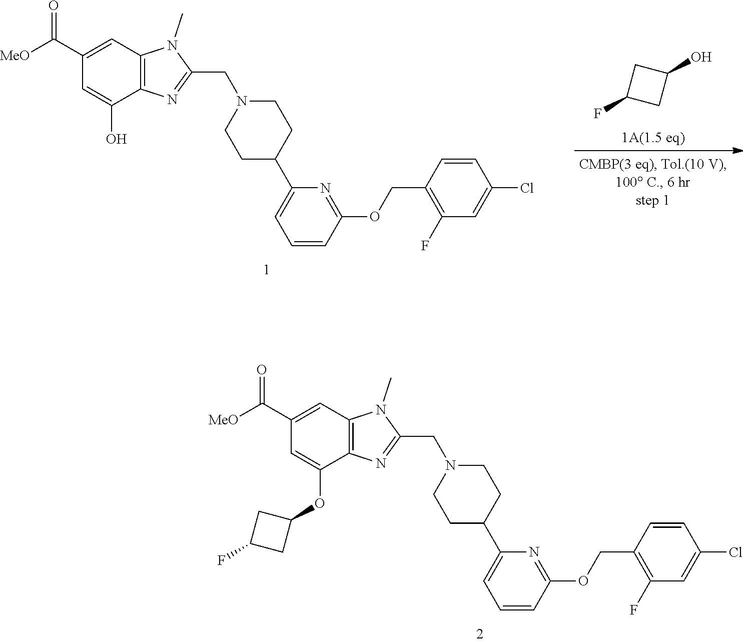 [SMILES: COC(=O)c1cc(O)c2nc(CN3CCC(c4cccc(OCc5ccc(Cl)cc5F)n4)CC3)n(C)c2c1 ; O[C@H]1C[C@@H](F)C1 ; COC(=O)c1cc(O[C@H]2C[C@H](F)C2)c2nc(CN3CCC(c4cccc(OCc5ccc(Cl)cc5F)n4)CC3)n(C)c2c1]

A mixture of 1 (100 mg, 185.53 μmol, 1 eq), 1A (25.07 mg, 278.29 μmol, 1.5 eq) and CMBP (134.33 mg, 556.59 μmol, 3 eq) in Toluene (1 mL) was degassed and purged with N₂ for 3 times, and then the mixture was stirred at 100° C. for 6 hr under N₂ atmosphere. LCMS (product: RT=1.743 min) showed the 1 was consumed completely. The reaction mixture was cooled down to 20° C. and quenched by addition H₂O 5 mL at 20° C., and extracted with MTBE 5 mL*4. The combined organic layers were washed with brine 4 mL*5, dried over Na₂SO₄, filtered and concentrated under reduced pressure to give a residue. The residue was purified by column chromatography (SiO₂, Petroleum ether/Ethyl acetate=5/1 to 0/1). 2 (90 mg, 147.28 μmol, 79.38% yield) was obtained as a red solid.

LCMS: RT=1.743 min, MS cal.: 610.2, [M+H]⁺=611.1

¹HNMR (400 MHz, DMSO-d6) δ=7.84 (d, J=1.2 Hz, 1H), 7.62 (t, J=7.6 Hz, 1H), 7.55 (t, J=8.4 Hz, 1H), 7.45 (dd, J=2.0, 10.0 Hz, 1H), 7.28 (dd, J=1.6, 8.0 Hz, 1H), 7.09 (d, J=1.2 Hz, 1H), 6.86 (d, J=7.6 Hz, 1H), 6.66 (d, J=8.4 Hz, 1H), 5.49-5.40 (m, 1H), 5.35 (s, 2H), 5.33-5.23 (m, 1H), 3.92 (s, 3H), 3.87 (s, 3H), 3.81 (s, 2H), 2.92 (d, J=11.2 Hz, 2H), 2.78-2.66 (m, 2H), 2.63-2.54 (m, 3H), 2.20 (t, J=10.4 Hz, 2H), 1.85-1.62 (m, 4H)

### General Procedure for Preparation of Compound 353 (3):

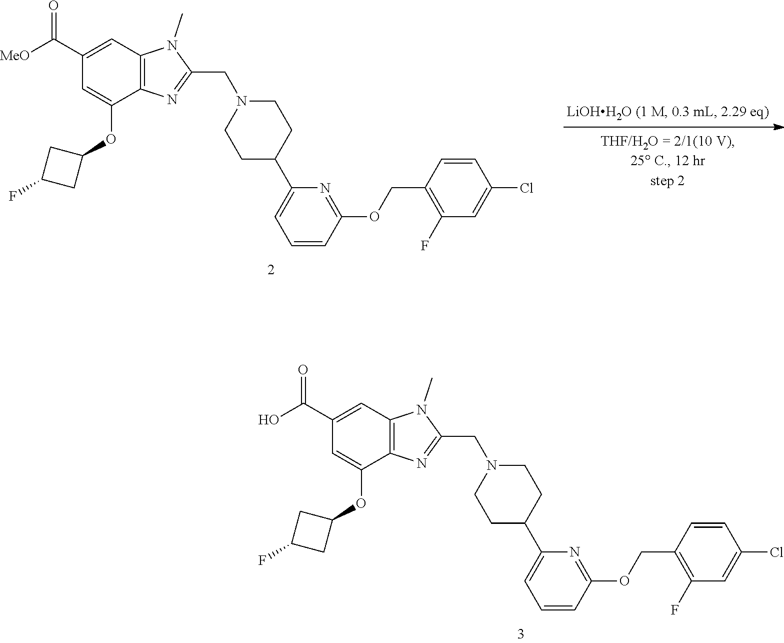 [SMILES: COC(=O)c1cc(O[C@H]2C[C@H](F)C2)c2nc(CN3CCC(c4cccc(OCc5ccc(Cl)cc5F)n4)CC3)n(C)c2c1 ; Cn1c(CN2CCC(c3cccc(OCc4ccc(Cl)cc4F)n3)CC2)nc2c(O[C@H]3C[C@H](F)C3)cc(C(=O)O)cc21]

To a solution of 2 (80 mg, 130.92 μmol, 1 eq) in THF (0.7 mL) was added LiOH·H₂O (1 M, 0.3 mL, 2.29 eq). The mixture was stirred at 25° C. for 12 hr under N₂ atmosphere. LCMS (product: RT=1.243 min) showed the 2 was consumed completely. The reaction mixture was filtered, and the filtrate was purified by prep-HPLC (column: Waters Xbridge Prep OBD C18 150*40 mm*10 um; mobile phase: [H₂O (10 mM NH₄HCO₃)-ACN]; gradient: 20%-70% B over 8.0 min). 2-((4-(6-((4-Chloro-2-fluorobenzyl)oxy)pyridin-2-yl)piperidin-1-yl)methyl)-4-((1r,3r)-3-fluorocyclobutoxy)-1-methyl-1H-benzo[d]imidazole-6-carboxylic acid (22.48 mg, 36.36 μmol, 27.78% yield, 96.58% purity) was obtained as a white solid.

LCMS: RT=2.768 min, MS cal.: 596.2, [M+H]⁺=597.2

HPLC: RT=12.162 min

¹H NMR (400 MHz, DMSO-d6) δ=12.98-12.66 (m, 1H), 7.81 (s, 1H), 7.62 (t, J=7.6 Hz, 1H), 7.55 (t, J=8.4 Hz, 1H), 7.45 (dd, J=1.6, 10.0 Hz, 1H), 7.31-7.24 (m, 1H), 7.08 (s, 1H), 6.86 (d, J=7.2 Hz, 1H), 6.66 (d, J=8.4 Hz, 1H), 5.49-5.40 (m, 1H), 5.35 (s, 2H), 5.33-5.28 (m, 1H), 5.25 (dt, J=4.0, 6.8 Hz, 1H), 3.91 (s, 3H), 3.82 (s, 2H), 2.92 (br d, J=10.8 Hz, 2H), 2.78-2.65 (m, 2H), 2.64-2.53 (m, 3H), 2.30-2.09 (m, 2H), 1.86-1.62 (m, 4H)

### Example A93—Preparation of Compound 354. 2-((4-(2-((4-Chloro-2-fluorobenzyl)oxy)-5-fluoropyrimidin-4-yl)piperidin-1-yl)methyl)-4-(difluoromethoxy)-1-methyl-1H-benzo[d]imidazole-6-carboxylic acid (Compound 354) (5)

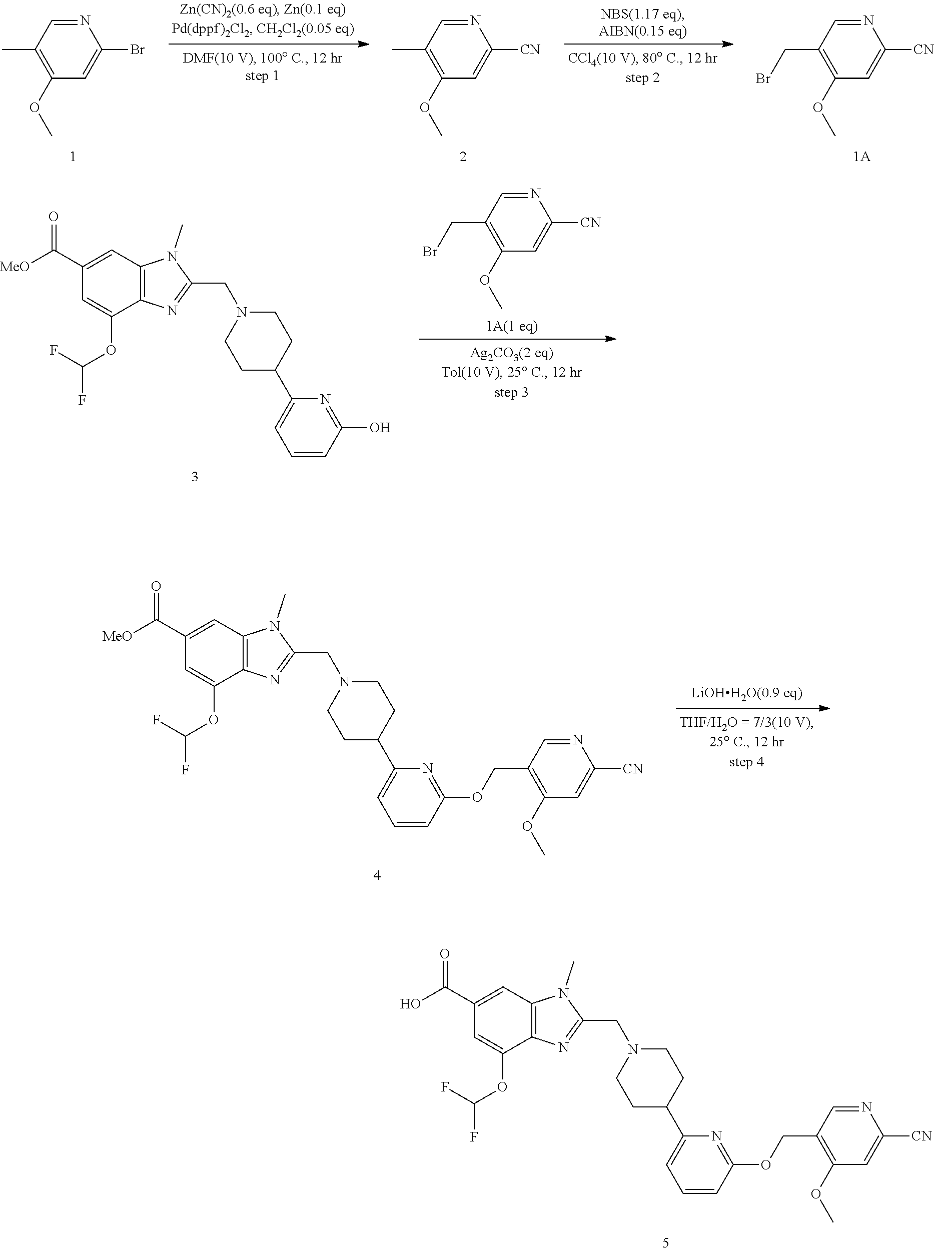

### General Procedure for Preparation of Intermediate 2:

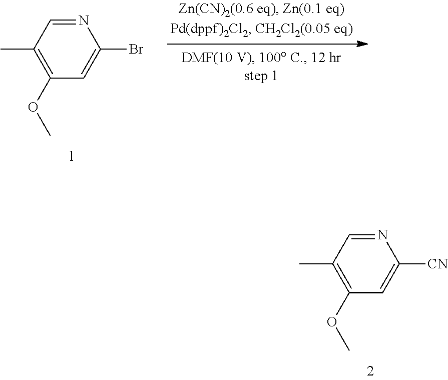

To a mixture of 1 (200 mg, 989.86 μmol, 1 eq) in DMF (2 mL) was added Zn (CN)₂ (69.74 mg, 593.92 μmol, 37.70 μL, 0.6 eq), Zn (6.47 mg, 98.99 μmol, 0.1 eq) and Pd(dppf)Cl₂·CH₂Cl₂ (40.42 mg, 49.49 μmol, 0.05 eq) at 20° C. The mixture was degassed and purged with N₂ for 3 times. The mixture was stirred at 100° C. for 12 hours. The color changes to black. TLC (SiO₂, PE:EA=5:1, R_{f}=0.50) showed the reaction was completed. The suspension was filtered through a pad of Celite and filter cake was washed with EtOAc 5 mL*3. The combined filtrates were diluted with H₂O 30 mL and extracted with EtOAc 60 mL 20 mL*3. The combined organic layers were washed with brine 45 mL, dried over Na₂SO₄, filtered, and concentrated under reduced pressure to give a residue. The reaction was purified by prep-TLC (SiO₂, PE:EA=5:1, R (=0.45). 2 (92 mg, 603.50 μmol, 60.97% yield) was obtained as a white solid.

HPLC: RT=2.021 min, purity: 97.19%

¹H NMR (400 MHz, MeOH-d4) δ=8.31 (s, 1H), 7.51 (s, 1H), 3.98 (s, 3H), 2.24 (s, 3H)

### General Procedure for Preparation of Intermediate 1A:

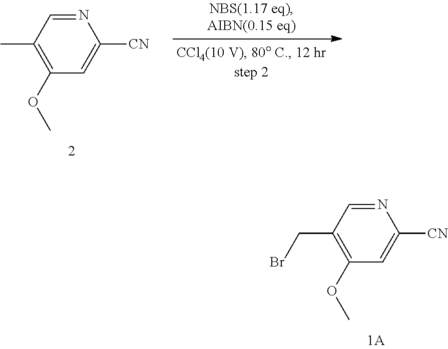

To a mixture of 2 (92 mg, 620.94 μmol, 1 eq) in CCl₄ (2 mL) was added NBS (129.31 mg, 726.50 μmol, 1.17 eq) and AIBN (15.29 mg, 93.14 μmol, 0.15 eq). The mixture was stirred at 80° C. for 12 hours. The color changes to orange. TLC (SiO₂, PE:EA=5:1, R_{f}=0.40) showed the reaction was completed. The reaction mixture was quenched by addition water 10 mL, and then extracted with DCM 30 mL (10 mL*3). The combined organic layers were washed with brine 30 mL, dried over Na₂SO₄, filtered, and concentrated under reduced pressure to give a residue. The residue was purified by prep-TLC (SiO₂, PE:EA=5:1, R_{f}=0.35). 1A (104 mg, 412.23 μmol, 66.39% yield) was obtained as a yellow solid.

HPLC: RT=2.293 min, purity: 89.09%

¹H NMR (400 MHz, MeOH-d4) δ=8.53 (s, 1H), 7.62 (s, 1H), 4.59 (s, 2H), 4.05 (s, 3H)

### General Procedure for Preparation of Intermediate 4:

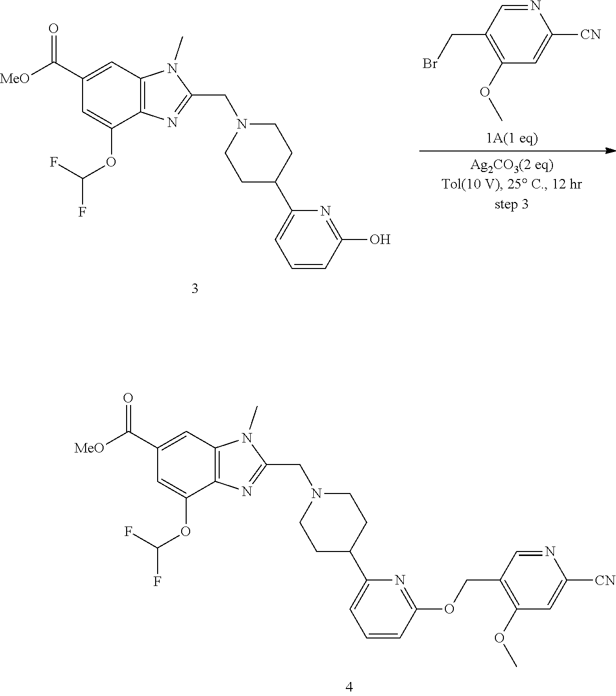 [SMILES: COC(=O)c1cc(OC(F)F)c2nc(CN3CCC(c4cccc(O)n4)CC3)n(C)c2c1 ; COc1cc(C#N)ncc1CBr ; COC(=O)c1cc(OC(F)F)c2nc(CN3CCC(c4cccc(OCc5cnc(C#N)cc5OC)n4)CC3)n(C)c2c1]

To a mixture 3 (100 mg, 223.99 μmol, 1 eq), 1A (101.72 mg, 447.98 μmol, 2 eq) and Ag₂CO₃ (123.53 mg, 447.98 μmol, 20.33 μL, 2 eq) in Toluene (2 mL) was stirred at 100° C. for 12 hours under N₂. The color changes to black. TLC (SiO₂, PE:EA=1:1, R_{f}=0.50) showed the reaction was completed. The reaction mixture was quenched by addition water 5 mL, and then extracted with EA 15 mL (5 mL*3). The combined organic layers were washed with brine 20 mL, dried over Na₂SO₄, filtered and concentrated under reduced pressure to give a residue. The residue was purified by prep-TLC (SiO₂, PE:EA=1:1, R_{f}=0.45). 4 (90 mg, 144.80 μmol, 64.64% yield) was obtained as a white solid.

HPLC: RT=2.949 min, purity: 95.34%

### General Procedure for Preparation of Compound 354 (5):

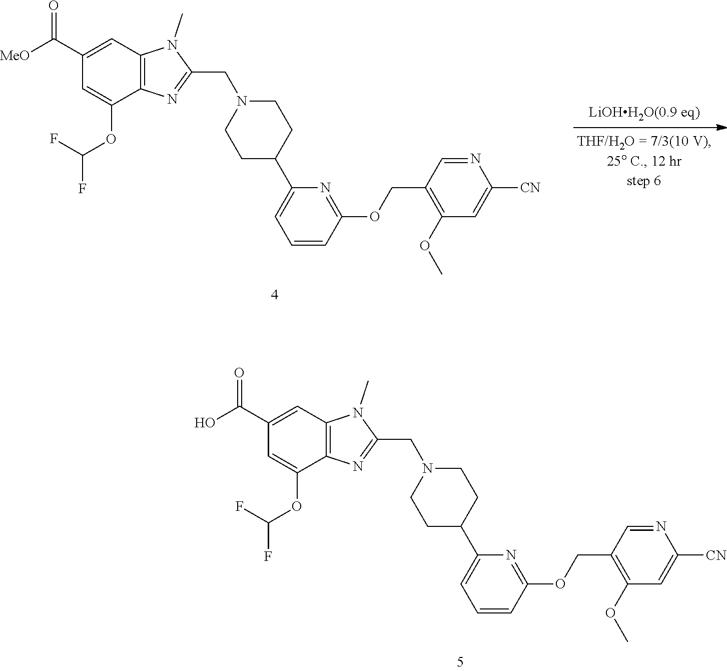 [SMILES: COC(=O)c1cc(OC(F)F)c2nc(CN3CCC(c4cccc(OCc5cnc(C#N)cc5OC)n4)CC3)n(C)c2c1 ; COc1cc(C#N)ncc1COc1cccc(C2CCN(Cc3nc4c(OC(F)F)cc(C(=O)O)cc4n3C)CC2)n1]

To a mixture 4 (80 mg, 135.00 μmol, 1 eq) and LiOH·H₂O (5.10 mg, 121.50 μmol, 0.9 eq) in THF (0.7 mL) and H₂O (0.3 mL) was stirred at 20° C. for 12 hours. The color changes to yellow. LCMS showed 78% of desired Compound 354 was detected. The mixture was concentrated in vacuum. The residue was purified by prep-HPLC (column: Waters Xbridge Prep OBD C18 150*40 mm*10 um; mobile phase: [H₂O (10 mM NH₄HCO₃)-ACN]; gradient: 30%-50% B over 8.0 min). 2-((4-(2-((4-Chloro-2-fluorobenzyl)oxy)-5-fluoropyrimidin-4-yl)piperidin-1-yl)methyl)-4-(difluoromethoxy)-1-methyl-1H-benzo[d]imidazole-6-carboxylic acid (28.16 mg, 48.03 μmol, 35.58% yield) was obtained as a white solid.

LCMS: RT=2.488 min, MS cal.: 578.2, [M+H]⁺=579.3.

HPLC: RT=9.793 min, purity: 98.69%

¹H NMR (400 MHz, DMSO-d6) δ=8.59 (s, 1H), 8.07 (d, J=1.2 Hz, 1H), 7.84 (s, 1H), 7.63 (t, J=74.4 Hz, 1H), 7.66-7.60 (m, 1H), 7.52 (s, 1H), 6.87 (d, J=7.6 Hz, 1H), 6.70 (d, J=8.0 Hz, 1H), 5.37 (s, 2H), 3.97 (d, J=4.8 Hz, 6H), 3.85 (s, 2H), 2.94 (d, J=10.8 Hz, 2H), 2.58 (dd, J=4.0, 11.2 Hz, 1H), 2.22 (t, J=11.2 Hz, 2H), 1.81-1.62 (m, 4H)

### Example A94—Preparation of Compound 355. 2-((4-(3-((4-Chloro-2-fluorobenzyl)oxy)pyridazin-4-yl)piperidin-1-yl)methyl)-4-(difluoromethoxy)-1-methyl-1H-benzo[d]imidazole-6-carboxylic acid (Compound 355) (7)

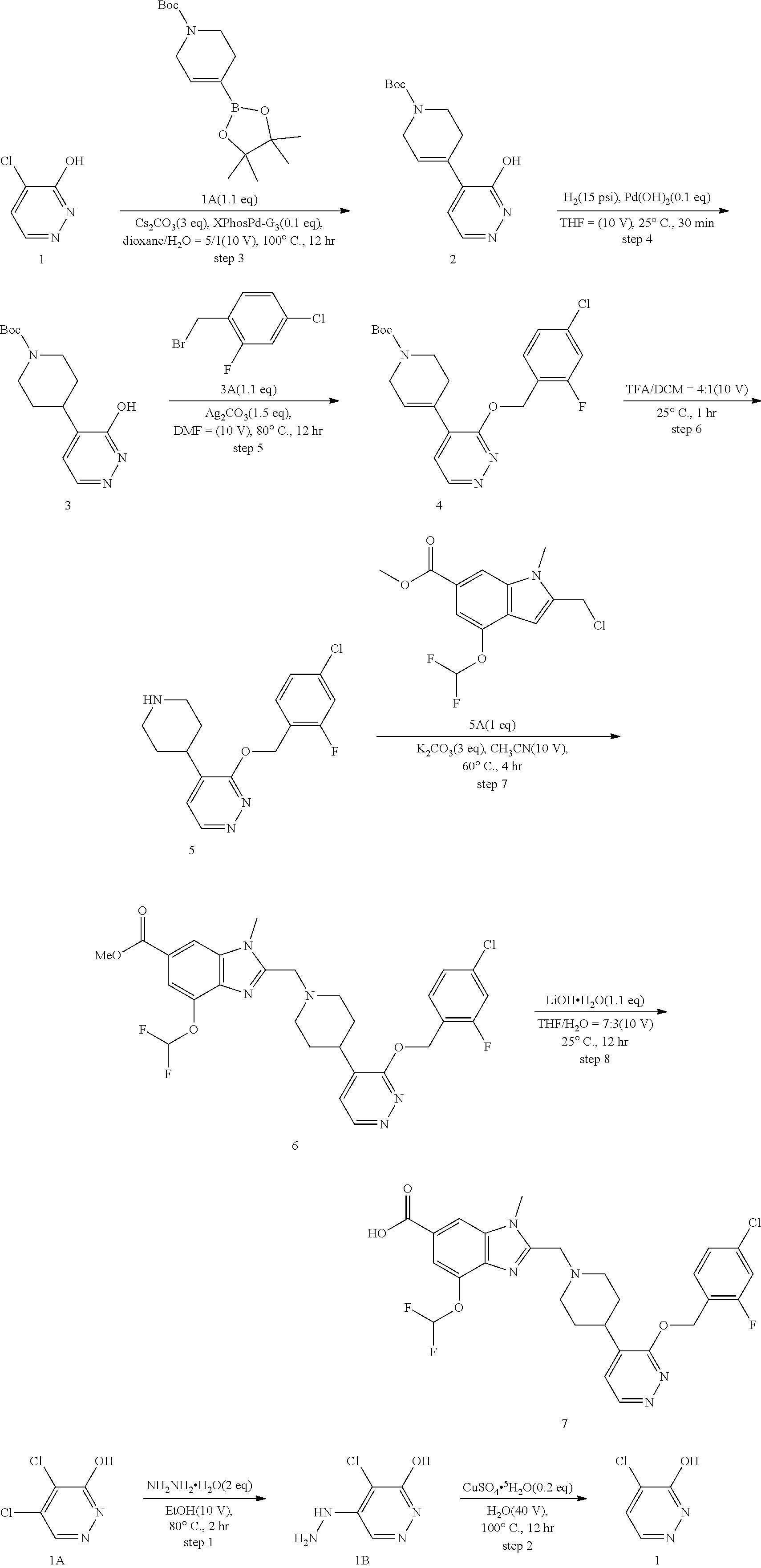

### General Procedure for Preparation of Intermediate 1B:

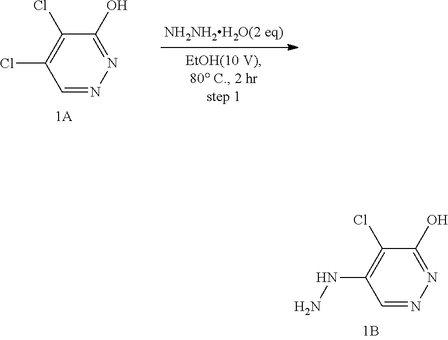 [SMILES: Oc1nncc(Cl)c1Cl ; NNc1cnnc(O)c1Cl]

To a mixture of 1A (3 g, 18.18 mmol, 1 eq) in EtOH (30 mL) was added NH₂NH₂·H₂O (3.18 g, 62.25 mmol, 3.08 mL, 98% purity, 3.42 eq) in one portion at 20° C. under N₂. The mixture was stirred at 80° C. for 2 hours. The solution of changed to cloudy. TLC (SiO₂, PE:EA=0:1, R_{f}=0.3) showed 1A was consumed completely and one new spot form. The residue was poured into ice-water (30 mL) and stirred for 5 min. The aqueous phase was extracted with ethyl acetate (20 mL*3). The combined organic phase was washed with brine (30 mL*2), dried with anhydrous Na₂SO₄, filtered, and concentrated in vacuum. The crude product was used to next step without purification. 1B (2 g, 12.46 mmol, 68.50% yield) as a yellow solid.

¹H NMR (400 MHz, MeOH-d4) δ=12.46 (br s, 1H), 8.08 (s, 1H), 7.97 (s, 1H), 4.58 (s, 2H)

### General Procedure for Preparation of Intermediate 1:

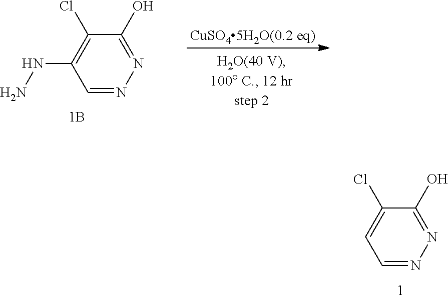 [SMILES: NNc1cnnc(O)c1Cl ; Oc1nnccc1Cl]

To a mixture of 1B (2 g, 12.46 mmol, 1 eq) in H₂O (80 mL) was added CuSO₄·5H₂O (622.03 mg, 2.49 mmol, 0.2 eq) in one portion at 20° C. under N₂. The mixture was stirred at 100° C. for 12 hours. The color of the solution changed black. LC-MS showed Compound 1B was consumed completely and one main peak with desired mass was detected. Filtrate, The mixture was cooled to 0° C., and an aq solution of NaOH was added until the pH was 4. The aqueous layer was thrice extracted with EtOAc (10 mL each). The combined extracts were dried (Na₂SO₄), filtered and evaporated. The remaining aqueous phase was adjusted to pH of 2 with conc. HCl and the solution was extracted with EtOAc six times. The extracts were combined, dried over Na₂SO₄, the mixture was concentrated in vacuum. The crude product was purified by prep-TLC (Petroleum ether:Ethyl acetate-0:1, R_{f}=0.5). 1 (600 mg, 4.60 mmol, 36.90% yield) as a white solid.

LCMS: RT=0.133 min, MS cal.: 130.0, [M+H−(t-Bu)]⁺=130.1.2

¹H NMR (400 MHz, MeOH-d4) δ=7.84 (d, J=4.4 Hz, 1H), 7.66 (d, J=4.4 Hz, 1H)

### General Procedure for Preparation of Intermediate 2:

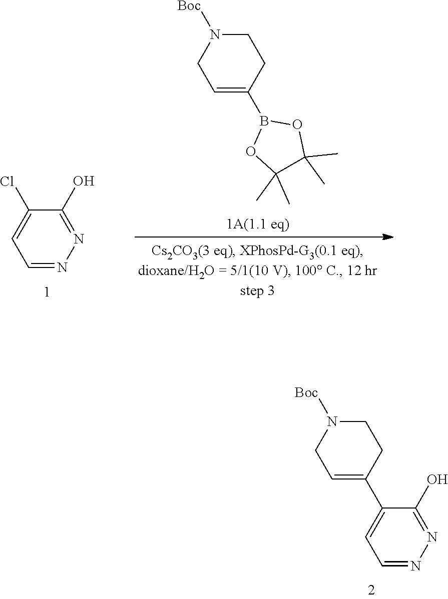 [SMILES: Oc1nnccc1Cl ; CC(C)(C)OC(=O)N1CC=C(B2OC(C)(C)C(C)(C)O2)CC1 ; CC(C)(C)OC(=O)N1CC=C(c2ccnnc2O)CC1]

To a mixture of 1 (600 mg, 4.60 mmol, 1 eq) and 1A (1.56 g, 5.06 mmol, 1.1 eq) in dioxane (10 mL) and H₂O (2 mL) was added Cs₂CO₃ (4.49 g, 13.79 mmol, 3 eq), XPhosPd-G₃ (389.07 mg, 459.66 μmol, 0.1 eq) in one portion at 20° C. under N₂. The mixture was de-gassed under reduced pressure and recharged with N₂. The mixture was stirred at 100° C. for 12 hours. LC-MS showed Compound 3 was consumed completely and one main peak with desired mass was detected. Filtrate, The mixture was concentrated in vacuum. The residue was purified by silica gel chromatography (column height: 250 mm, diameter: 100 mm, 100-200 mesh silica gel, Petroleum ether/Ethyl acetate=100/0, 60/40). 2 (500 mg, 1.80 mmol, 39.22% yield) as a white solid.

LCMS: RT=1.010 min, MS cal.: 277.1, [M+Na]⁺=300.2

¹H NMR (400 MHz, MeOH-d4) δ=7.88 (d, J=4.4 Hz, 1H), 7.29 (d, J=4.4 Hz, 1H), 7.11 (br s, 1H), 4.11 (br s, 2H), 3.62 (br t, J=5.6 Hz, 2H), 2.48 (br d, J=1.6 Hz, 2H), 1.48 (s, 9H)

### General Procedure for Preparation of Intermediate 3:

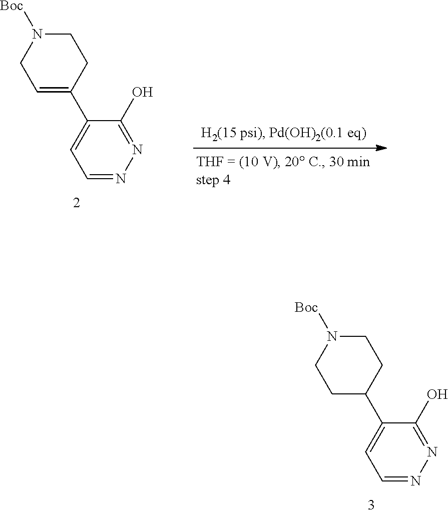 [SMILES: CC(C)(C)OC(=O)N1CC=C(c2ccnnc2O)CC1 ; CC(C)(C)OC(=O)N1CCC(c2ccnnc2O)CC1]

To a mixture of 2 (500 mg, 1.80 mmol, 1 eq) in THF (10 mL) was added Pd(OH)₂ (126.60 mg, 90.15 μmol, 10% purity, 0.05 eq), in one portion at 20° C. under N₂. The mixture was de-gassed under reduced pressure and recharged with N₂. Then, the mixture was de-gassed under reduced pressure and recharged with H₂ (15 Psi). The mixture was stirred at 20° C. for 2 hours (15 Psi). LCMS showed reactant 1 was consumed completely and a major peak was desired product. Filtrate, the mixture was filtered and concentrated in vacuum. The crude product was used to next step without purification. 3 (450 mg, 1.61 mmol, 89.35% yield) as a gray solid.

LCMS: RT=0.382 min, MS cal.: 279.2, [M+Na]⁺=302.2

¹H NMR (400 MHz, CHCl3-d) δ=7.85 (d, J=4.0 Hz, 1H), 7.23 (dd, J=0.8, 4.0 Hz, 1H), 4.21 (br d, J=13.2 Hz, 2H), 3.03-2.81 (m, 3H), 1.91-1.88 (m, 1H), 1.87-1.85 (m, 1H), 1.56-1.47 (m, 2H), 1.47 (s, 9H)

### General Procedure for Preparation of Intermediate 4:

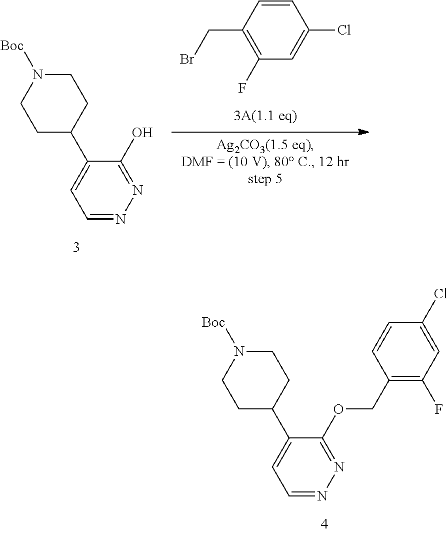

To a mixture of 3 (200 mg, 715.99 μmol, 1 eq) and 3A (176.00 mg, 787.59 μmol, 1.1 eq) in DMF (3 mL) was added Ag₂CO₃ (394.86 mg, 1.43 mmol, 64.98 μL, 2 eq) in one portion at 20° C. under N₂. The mixture was de-gassed under reduced pressure and recharged with N₂. The mixture was stirred at 100° C. 2 hours. LCMS showed 3 was consumed completely and one main peak with desired mass was detected. Filtrated, the mixture was concentrated in vacuum. The crude product was purified by prep-TLC (Petroleum ether:Ethyl acetate=1:1,Rr-0.4). 4 (130 mg, 308.14 μmol, 43.04% yield) as a white solid.

LCMS: RT=0.566 min, MS cal.: 421.2, [M+H]⁺=422.2

¹H NMR (400 MHz, CHCl3-d) δ=8.75 (d, J=4.8 Hz, 1H), 7.58 (t, J=8.0 Hz, 1H), 7.48 (d, J=4.8 Hz, 1H), 7.30-7.23 (m, 2H), 5.61 (s, 2H), 4.20 (br d, J=13.2 Hz, 2H), 3.01 (tt, J=3.2, 12.0 Hz, 1H), 2.86 (br d, J=1.2 Hz, 2H), 1.86 (br d, J=12.4 Hz, 2H), 1.63-1.52 (m, 2H), 1.45 (s, 9H)

### General Procedure for Preparation of Intermediate 5:

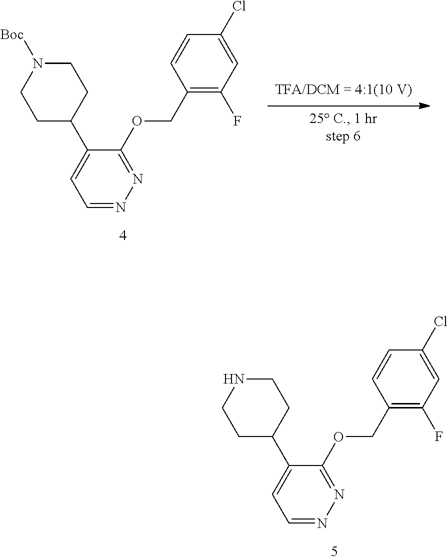 [SMILES: CC(C)(C)OC(=O)N1CCC(c2ccnnc2OCc2ccc(Cl)cc2F)CC1 ; "25° C., 1 hrstep 6" ; Fc1cc(Cl)ccc1COc1nnccc1C1CCNCC1]

To a mixture 4 (130 mg, 308.14 μmol, 1 eq) in DCM (1.6 mL) was added TFA (702.69 mg, 6.16 mmol, 457.78 μL, 20 eq) in one portion at 20° C. under N₂. The mixture was stirred at 20° C. 2 hours. LC-MS showed 4 was consumed completely and one main peak with desired mass was detected. To a mixture was added Saturated sodium carbonate solution to PH=9˜10. The aqueous phase was extracted with DCM (5 mL*3). The combined organic phase was washed with brine (10 mL*2), dried with anhydrous Na₂SO₄, filtered and concentrated in vacuum. The crude product was used to next step without purification. 5 (90 mg, 279.70 μmol, 90.77% yield) as a white solid.

LCMS: RT=0.325 min, MS cal.: 321.1, [M+H]⁺=322.0

¹H NMR (400 MHz, CHCl3-d) δ=8.75 (d, J=4.8 Hz, 1H), 7.58 (t, J=8.0 Hz, 1H), 7.48 (d, J=4.8 Hz, 1H), 7.30-7.22 (m, 2H), 5.61 (s, 2H), 3.13 (br d, J=12.4 Hz, 2H), 3.02-2.92 (m, 1H), 2.70 (dt, J=2.4, 12.8 Hz, 2H), 1.86 (br d, J=12.4 Hz, 2H), 1.58 (dq, J=3.6, 12.4 Hz, 2H)

### General Procedure for Preparation of Intermediate 6:

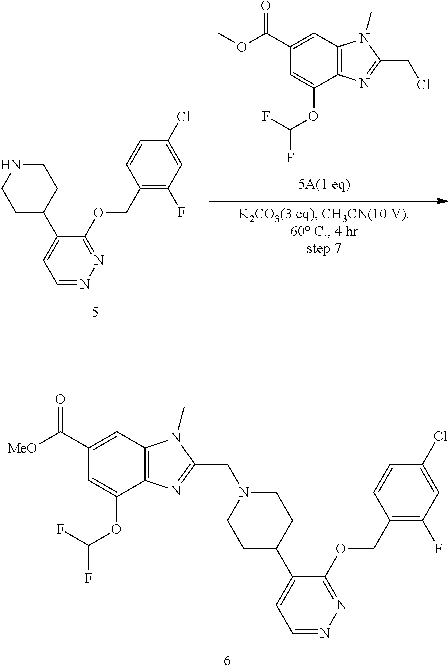 [SMILES: Fc1cc(Cl)ccc1COc1nnccc1C1CCNCC1 ; COC(=O)c1cc(OC(F)F)c2nc(CCl)n(C)c2c1 ; "K2CO3(3 eq), CH3CN(10 V).60° C., 4 hrstep 7" ; COC(=O)c1cc(OC(F)F)c2nc(CN3CCC(c4ccnnc4OCc4ccc(Cl)cc4F)CC3)n(C)c2c1]

To a mixture of 5 (90 mg, 279.70 μmol, 1 eq) and 5A (85.22 mg, 279.70 μmol, 1 eq) in ACN (1 mL) was added K₂CO₃ (115.97 mg, 839.09 μmol, 3 eq) in one portion at 20° C. The mixture was de-gassed under reduced pressure and recharged with N₂. The mixture was stirred at 60° C. for 2 hours. The solution changed to cloudy. LCMS showed Compound 1 was consumed completely and one main peak with desired mass was detected. Filtrate, the mixture was concentrated in vacuum. The crude product was purified by prep-TLC (Petroleum ether:Ethyl acetate=1:1, R_{f}=0.3). 6 (110 mg, 186.44 μmol, 66.66% yield) as a white solid.

LCMS: RT=0.427 min, MS cal.: 589.2, [M+H]⁺=590.3

¹H NMR (400 MHz, CHCl3-d) δ=8.81 (d, J=4.8 Hz, 1H), 8.11 (d, J=1.2 Hz, 1H), 7.64 (t, J=74.4 Hz, 1H), 7.61 (t, J=8.4 Hz, 1H), 7.54-7.51 (m, 2H), 7.35 (dd, J=2.0, 8.4 Hz, 1H), 5.56 (s, 2H), 3.93 (s, 3H), 3.90 (s, 3H), 3.85 (s, 2H), 2.94 (br d, J=11.2 Hz, 2H), 2.72 (br t, J=12.0 Hz, 1H), 2.21 (br t, J=10.8 Hz, 2H), 1.76 (br d, J=11.6 Hz, 2H), 1.69-1.56 (m, 2H)

### General Procedure for Preparation of Compound 355 (7):

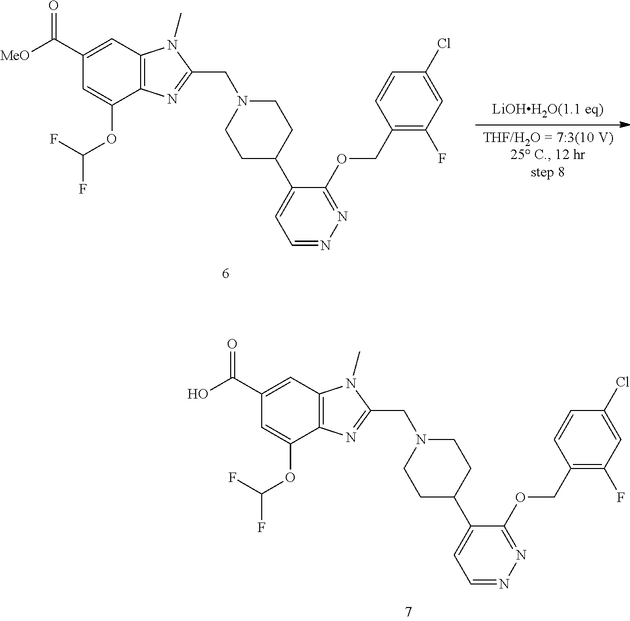

To a mixture of 6 (110 mg, 186.44 μmol, 1 eq) in THF (0.7 mL) and H₂O (0.3 mL) was added LiOH·H₂O (8.61 mg, 205.09 μmol, 1.1 eq) in one portion at 20° C. The mixture was stirred at 20° C. for 12 hours. LC-MS showed Compound 6 was consumed completely and one main peak with desired mass was detected. The mixture was filtered and concentrated in vacuum. The crude product was purified by prep-HPLC (column: Waters Xbridge Prep OBD C₁₈ 150*40 mm*10 um; mobile phase: [H₂O (10 mM NH₄HCO₃)-ACN]; gradient: 10%-60% B over 8.0 min). 2-((4-(3-((4-Chloro-2-fluorobenzyl)oxy)pyridazin-4-yl)piperidin-1-yl)methyl)-4-(difluoromethoxy)-1-methyl-1H-benzo[d]imidazole-6-carboxylic acid (27.15 mg, 46.46 μmol, 24.92% yield, 98.561% purity) as a white solid.

LCMS: RT=2.449 min, MS cal.: 575.2, [M+H]⁺=576.2

HPLC: RT=9.429 min, purity: 98.56%

¹H NMR (400 MHz, MeOH-d4) δ=8.81 (d, J=4.8 Hz, 1H), 8.07 (s, 1H), 7.62 (t, J=74 Hz, 1H), 7.61-7.58 (m, 1H), 7.55-7.47 (m, 3H), 7.35 (br d, J=8.4 Hz, 1H), 5.56 (s, 2H), 3.92 (s, 3H), 3.84 (s, 2H), 2.94 (br d, J=10.8 Hz, 2H), 2.72 (br t, J=12.0 Hz, 1H), 2.20 (br t, J=11.2 Hz, 2H), 1.76 (br d, J=11.6 Hz, 2H), 1.68-1.55 (m, 2H)

### Example A95—Preparation of Compound 356. 2-((4-(4-((4-Chloro-2-fluorobenzyl)oxy)-5-fluoropyrimidin-2-yl)piperidin-1-yl)methyl)-4-(difluoromethoxy)-1-methyl-1H-benzo[d]imidazole-6-carboxylic acid (Compound 356) (7)

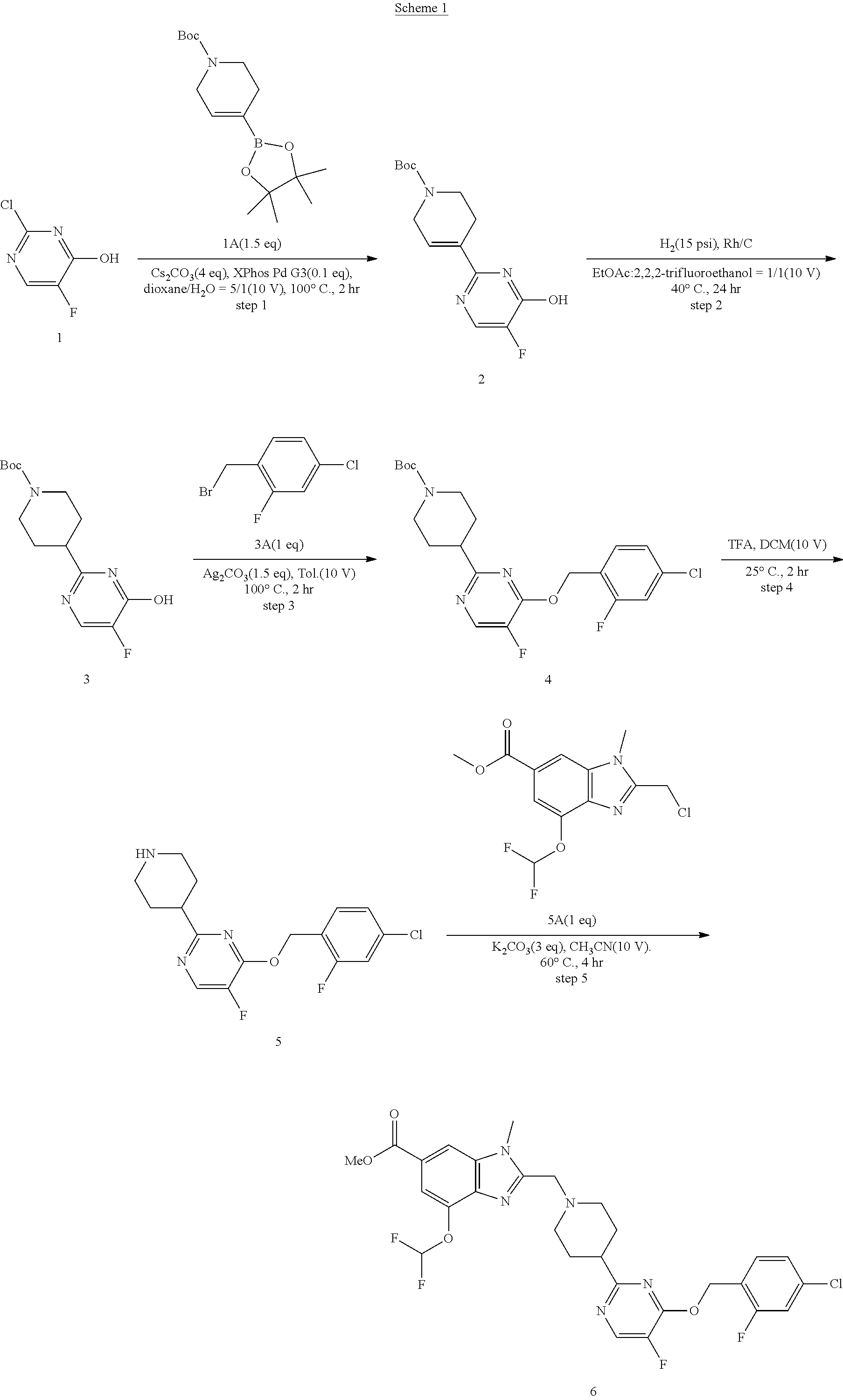

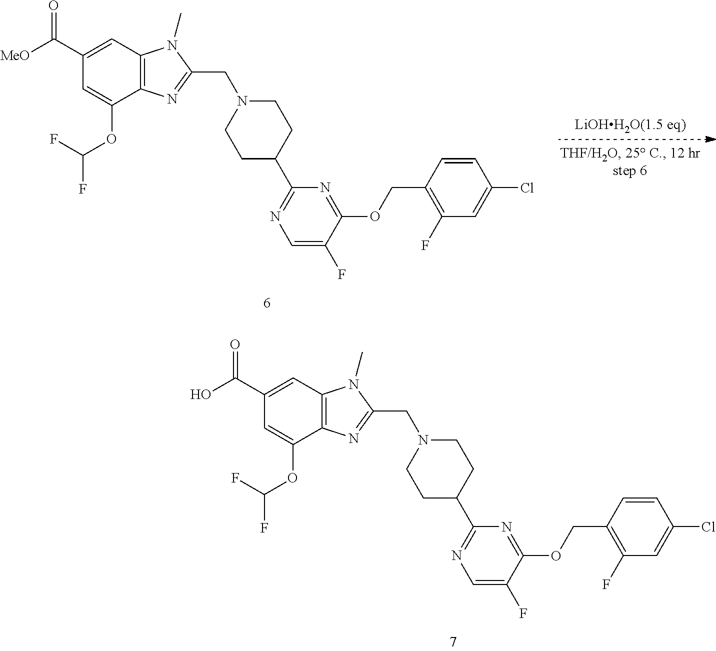 [SMILES: COC(=O)c1cc(OC(F)F)c2nc(CN3CCC(c4ncc(F)c(OCc5ccc(Cl)cc5F)n4)CC3)n(C)c2c1 ; "LiOH·H2O(1.5 eq)" ; "THF/H2O, 25° C., 12 hrstep 6" ; Cn1c(CN2CCC(c3ncc(F)c(OCc4ccc(Cl)cc4F)n3)CC2)nc2c(OC(F)F)cc(C(=O)O)cc21]

### General Procedure for Preparation of Intermediate 2:

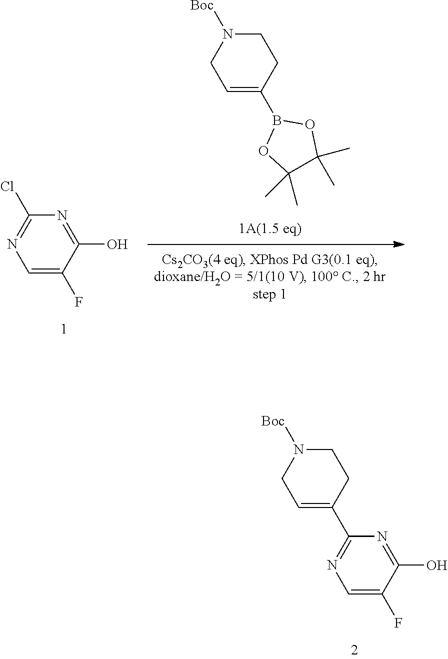 [SMILES: Oc1nc(Cl)ncc1F ; CC(C)(C)OC(=O)N1CC=C(B2OC(C)(C)C(C)(C)O2)CC1 ; CC(C)(C)OC(=O)N1CC=C(c2ncc(F)c(O)n2)CC1]

To a solution of 1 (1 g, 6.73 mmol, 1 eq) in dioxane (10 mL) and H₂O (2 mL) was added Cs₂CO₃ (8.77 g, 26.93 mmol, 4 eq), XPhos Pd G3 (569.91 mg, 673.30 μmol, 0.1 eq) and 1A (3.12 g, 10.10 mmol, 1.5 eq). The mixture was stirred at 100° C. for 2 hr. LCMS showed 1 was consumed completely and one main peak with desired mass was detected. The reaction mixture was diluted with H₂O 15 mL and extracted with EtOAc 60 mL (20 mL*3). The combined organic layers were washed with brine 10 mL, dried over Na₂SO₄, filtered and concentrated under reduced pressure to give a residue. The residue was purified by column chromatography (SiO₂, PE/EtOAc=1/0 to 3/2). 2 (780 mg, 2.25 mmol, 33.34% yield) was obtained as a white solid.

LCMS: RT=0.881 min, MS cal.: 295.1/296.1, [M−1]⁻=294.1/295.1

¹HNMR (400 MHz, CHCl3-d) δ=7.97 (d, J=2.4 Hz, 1H), 6.92-6.83 (m, 1H), 4.20 (br d, J=2.4 Hz, 2H), 3.66-3.56 (m, 2H), 2.65-2.57 (m, 2H), 1.50 (s, 9H)

### General Procedure for Preparation of Intermediate 3:

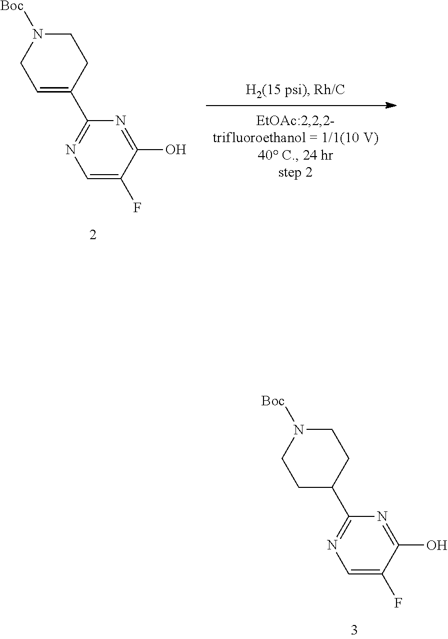 [SMILES: CC(C)(C)OC(=O)N1CC=C(c2ncc(F)c(O)n2)CC1 ; CC(C)(C)OC(=O)N1CCC(c2ncc(F)c(O)n2)CC1]

Equip a 100 mL one-necked round bottom flask, thermometer, H₂ balloon. EtOAc (1.5 mL) and 2,2,2-trifluoroethanol (1.5 mL) was charged to the 100 mL one-necked round bottom flask, then 2 (300 mg, 1.02 mmol, 1 eq) was added to the mixture at 25° C. Rh/C (30 mg) was added to the reaction mixture at 25° C. After the addition, the mixture was stirred at 40° C. for 12 hr at 15 psi. After monitoring, the mixture was added Rh/C (50 mg). After the addition, the mixture was stirred at 40° C. for 12 hr. LCMS showed 2 was consumed completely and one main peak with desired mass was detected. The reaction mixture was filtered, and the filter cake was washed with EtOAc. 3 (320 mg, crude) was obtained as a white solid.

LCMS: RT=0.913 min, MS cal.: 297.1/298.2, [M−1]⁻=296.0/297.0

¹HNMR (400 MHz, CHCl3-d) δ=7.94 (d, J=2.4 Hz, 1H), 7.95-7.88 (m, 1H), 4.31-4.11 (m, 2H), 2.89-2.76 (m, 2H), 1.96-1.89 (m, 2H), 1.88-1.72 (m, 3H), 1.47 (s, 9H)

### General Procedure for Preparation of Intermediate 4:

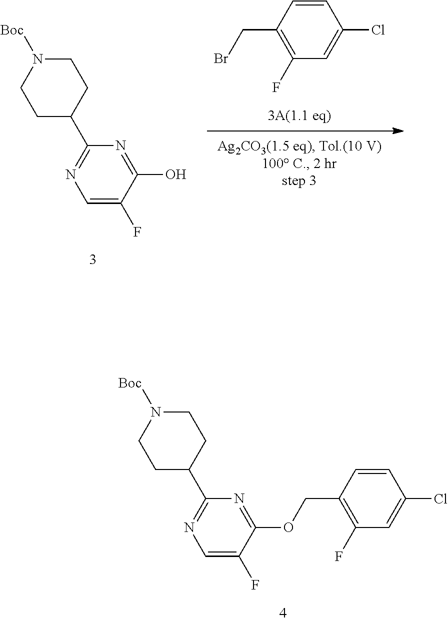 [SMILES: CC(C)(C)OC(=O)N1CCC(c2ncc(F)c(O)n2)CC1 ; Fc1cc(Cl)ccc1CBr ; CC(C)(C)OC(=O)N1CCC(c2ncc(F)c(OCc3ccc(Cl)cc3F)n2)CC1]

To a solution of 3 (260 mg, 874.47 μmol, 1 eq) in Toluene (2.6 mL) was added Ag₂CO₃ (361.69 mg, 1.31 mmol, 59.52 μL, 1.5 eq) and 3A (214.96 mg, 961.91 μmol, 1.1 eq). The mixture was stirred at 100° C. for 2 hr. LCMS showed 3 was consumed completely. Several new peaks were shown on LCMS and 22% of desired 4 was detected. The reaction mixture was diluted with H₂O 5 mL and extracted with EtOAc 30 mL (10 mL*3). The combined organic layers were washed with brine 10 mL, dried over Na₂SO₄, filtered, and concentrated under reduced pressure to give a residue. The residue was purified by column chromatography (SiO₂, PE/EtOAc=1/0 to 3/1). 4 (110 mg, 167.54 μmol, 19.16% yield) was obtained as a white solid.

LCMS: RT=2.397 min, MS cal.: 439.1/441.1, [M−55]⁺=384.2/386.2

¹HNMR (400 MHz, CHCl3-d) δ=8.28-8.25 (m, 1H), 7.48-7.36 (m, 1H), 7.19-7.14 (m, 2H), 5.53 (s, 2H), 4.27-4.14 (m, 2H), 2.97-2.82 (m, 3H), 1.96 (br d, J=12.8 Hz, 2H), 1.83-1.70 (m, 2H), 1.49 (s, 9H)

### General Procedure for Preparation of Intermediate 5:

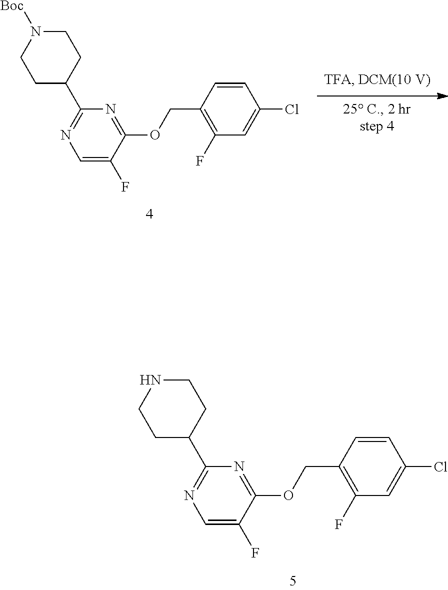 [SMILES: CC(C)(C)OC(=O)N1CCC(c2ncc(F)c(OCc3ccc(Cl)cc3F)n2)CC1 ; "25° C., 2 hrstep 4" ; Fc1cc(Cl)ccc1COc1nc(C2CCNCC2)ncc1F]

To a solution of 4 (100 mg, 227.33 μmol, 1 eq) in DCM (1 mL) was added TFA (767.50 mg, 6.73 mmol, 0.5 mL, 29.61 eq). The mixture was stirred at 25° C. for 2 hr. LCMS showed 4 was consumed completely and one main peak with desired mass was detected. The reaction mixture was concentrated under reduced pressure. 5 (100 mg, crude, TFA) was obtained as a yellow oil.

LCMS: RT=0.399 min, MS cal.: 339.1/341.1, [M+1]⁺=340.1/342.1

### General Procedure for Preparation of Intermediate 6:

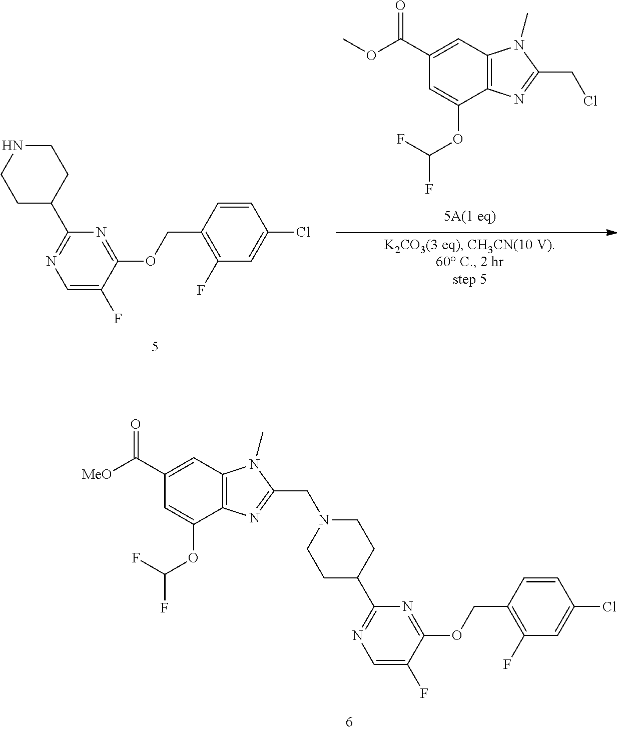 [SMILES: Fc1cc(Cl)ccc1COc1nc(C2CCNCC2)ncc1F ; COC(=O)c1cc(OC(F)F)c2nc(CCl)n(C)c2c1 ; "K2CO3(3 eq), CH3CN(10 V).60° C., 2 hrstep 5" ; COC(=O)c1cc(OC(F)F)c2nc(CN3CCC(c4ncc(F)c(OCc5ccc(Cl)cc5F)n4)CC3)n(C)c2c1]

To a solution of 5 (100 mg, 220.37 μmol, 1 eq, TFA) in CH₃CN (1 mL) was added K₂CO₃ (91.37 mg, 661.10 μmol, 3 eq) and 5A (67.14 mg, 220.37 μmol, 1 eq). The mixture was stirred at 60° C. for 2 hr. LCMS showed 5 was consumed completely and one main peak with desired mass was detected. The reaction mixture was diluted with H₂O 5 mL and extracted with EtOAc 10 mL (5 mL*2), the combined organic layers were washed with brine 10 mL, dried over Na₂SO₄, filtered, and concentrated under reduced pressure to give a residue. The residue was purified by column chromatography (SiO₂, PE/EtOAc=1/0 to 1/1). 6 (70 mg, 110.53 μmol, 50.16% yield) was obtained as a yellow solid.

LCMS: RT=1.632 min, MS cal.: 607.2/609.2, [M+1]⁺=608.1/610.1

¹HNMR (400 MHz, MeOH-d4) δ=8.33 (d, J=3.2 Hz, 1H), 8.15 (s, 1H), 7.68 (s, 1H), 7.57-7.52 (m, 1H), 7.45 (s, 1H), 7.30-7.22 (m, 3H), 7.08-7.07 (m, 1H), 5.57 (s, 2H), 4.03 (s, 3H), 3.96 (s, 3H), 3.91 (s, 2H), 3.01 (br d, J=12.0 Hz, 2H), 2.86-2.77 (m, 1H), 2.33 (br d, J=2.8 Hz, 2H), 1.99-1.86 (m, 4H)

### General Procedure for Preparation of Compound 356 (7):

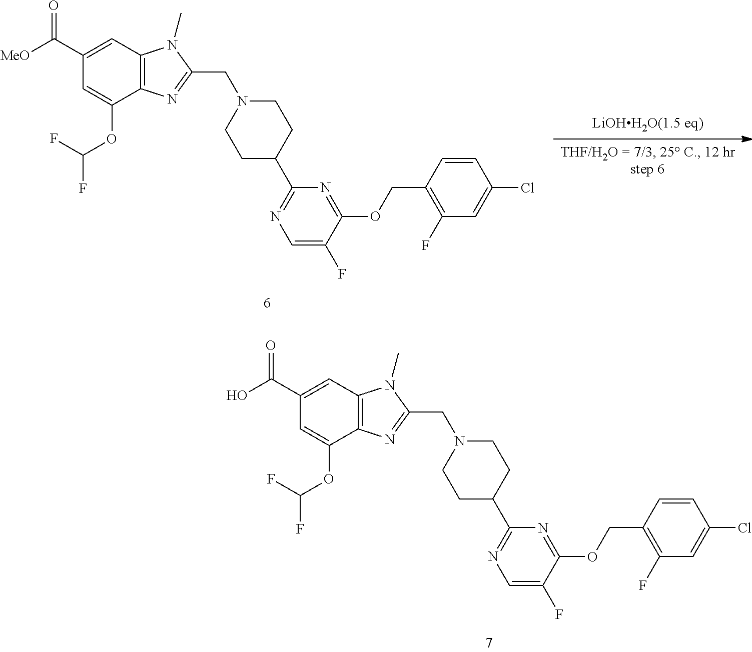

To a solution of 6 (60 mg, 98.69 μmol, 1 eq) in THF (0.7 mL) and H₂O (0.3 mL) was added LiOH·H₂O (6.21 mg, 148.03 μmol, 1.5 eq). The mixture was stirred at 25° C. for 12 hr. LCMS showed 6 was consumed completely and one main peak with desired mass was detected. The mixture was added THF 0.1 mL. The residue was purified by prep-HPLC (neutral condition: column: Waters Xbridge Prep OBD C18 150*40 mm*10 um; mobile phase: [H₂O (10 mM NH₄HCO₃)-ACN]; gradient: 20%-70% B over 8.0 min). 2-((4-(4-((4-Chloro-2-fluorobenzyl)oxy)-5-fluoropyrimidin-2-yl)piperidin-1-yl)methyl)-4-(difluoromethoxy)-1-methyl-1H-benzo[d]imidazole-6-carboxylic acid (26.32 mg, 43.87 μmol, 44.45% yield) was obtained as a white solid.

LCMS: RT=2.676 min, MS cal.: 593.1/595.1, [M+H]⁺=594.2/596.2

HPLC: RT=11.683 min

₁HNMR (400 MHz, MeOH-d4) δ=8.35-8.32 (m, 1H), 8.11 (d, J=1.2 Hz, 1H), 7.70-7.67 (m, 1H), 7.57-7.52 (m, 1H), 7.44-7.43 (m, 1H), 7.29-7.21 (m, 2H), 7.07-7.06 (m, 1H), 5.57 (s, 2H), 4.02 (s, 3H), 3.96 (s, 2H), 3.06 (br d, J=11.6 Hz, 2H), 2.89-2.79 (m, 1H), 2.39 (dt, J=2.4, 11.6 Hz, 2H), 2.03-1.86 (m, 4H)

### Example A96—Preparation of Compound 357. 2-((4-(6-((4-Chloro-2-fluorobenzyl)oxy)pyridin-2-yl)piperidin-1-yl)methyl)-4-(cyclopropylmethoxy)-1-methyl-1H-benzo[d]imidazole-6-carboxylic acid (Compound 357) (3)

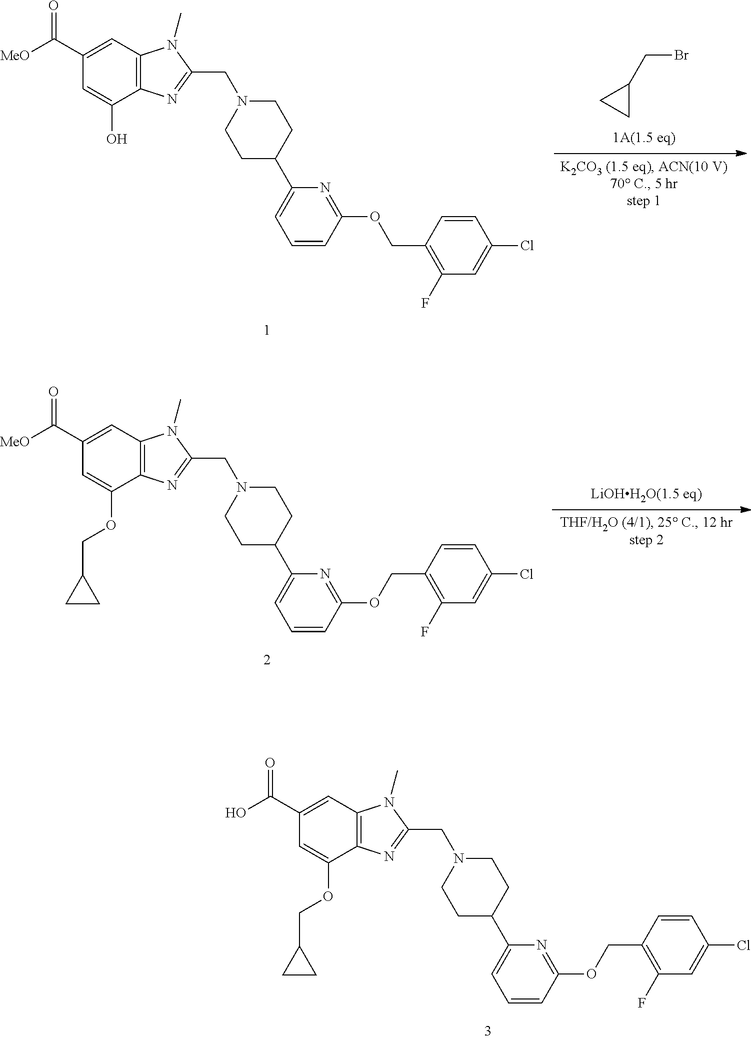

### General Procedure for Preparation of Intermediate 2:

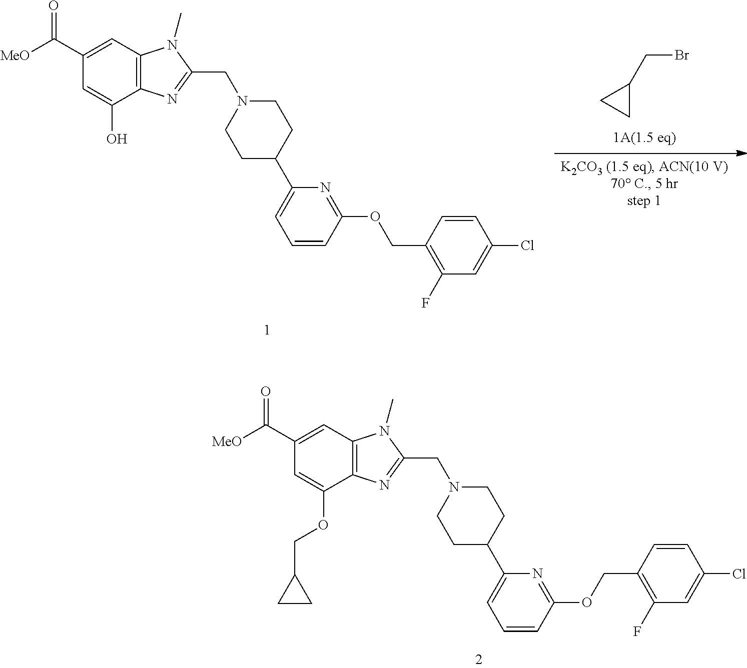

To a solution of 1 (100 mg, 185.53 μmol, 1 eq), 1A (37.57 mg, 278.29 μmol, 26.57 μL, 1.5 eq) in ACN (1 mL) was added K₂CO₃ (38.46 mg, 278.29 μmol, 1.5 eq). The mixture was stirred at 70° C. for 5 hr. LC-MS showed 1 was consumed completely and one main peak with desired mass was detected. The mixture was diluted with H₂O (5 mL), extracted with EtOAc (5 mL*3). The combined organic layer was washed with 10 mL brine, dried over Na₂SO₄, filtered and the filtrate was concentrated to give a crude product. The residue was purified by column chromatography (SiO₂, Petroleum ether:Ethyl acetate=1:1). 2 (45 mg, 75.87 μmol, 41% yield) was obtained as a white solid.

LCMS: RT=0.513 min, MS cal.: 592.2, 593.2, [M−H]⁺=593.2, 594.2

¹H NMR (400 MHz, CHCl3-d) δ=7.76 (s, 1H), 7.54-7.41 (m, 2H), 7.38 (s, 1H), 7.15-7.08 (m, 2H), 6.73 (d, J=7.2 Hz, 1H), 6.61 (d, J=8.0 Hz, 1H), 5.45-5.37 (m, 2H), 4.10 (d, J=7.2 Hz, 2H), 3.91 (s, 1H), 4.04-3.83 (m, 7H), 2.97 (d, J=10.0 Hz, 2H), 2.68-2.51 (m, 1H), 2.36-2.23 (m, 2H), 1.95-1.72 (m, 4H), 1.52-1.42 (m, 1H), 0.74-0.61 (m, 2H), 0.51-0.35 (m, 2H)

### General Procedure for Preparation of Compound 357 (3):

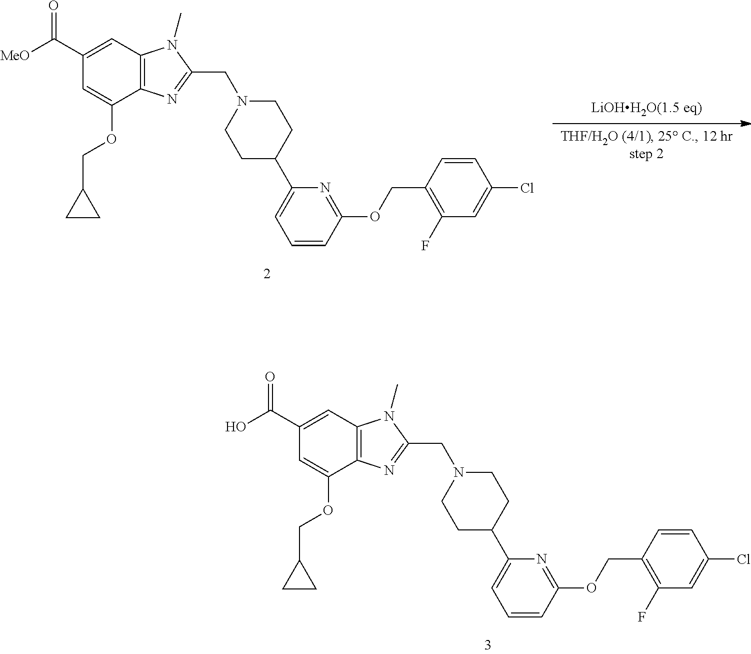 [SMILES: COC(=O)c1cc(OCC2CC2)c2nc(CN3CCC(c4cccc(OCc5ccc(Cl)cc5F)n4)CC3)n(C)c2c1 ; "LiOH·H2O(1.5 eq)" ; "THF/H2O (4/1), 25° C., 12 hrstep 2" ; Cn1c(CN2CCC(c3cccc(OCc4ccc(Cl)cc4F)n3)CC2)nc2c(OCC3CC3)cc(C(=O)O)cc21]

To a solution of 2 (40 mg, 67.44 μmol, 1 eq) in THF (0.4 mL) was added LiOH·H₂O (4.25 mg, 101.17 μmol, 1.5 eq) in H₂O (0.1 mL). The mixture was stirred at 25° C. for 12 hr. LC-MS showed 2 was consumed completely and one main peak with desired mass was detected. The reaction was combined for purification. The residue was purified by prep-HPLC (column: Waters Xbridge Prep OBD C18 150*40 mm*10 um; mobile phase: [H₂O (10 mM NH₄HCO₃)-ACN]; gradient: 30%-70% B over 8.0 min). 2-((4-(6-((4-Chloro-2-fluorobenzyl)oxy)pyridin-2-yl)piperidin-1-yl)methyl)-4-(cyclopropylmethoxy)-1-methyl-1H-benzo[d]imidazole-6-carboxylic acid (7.32 mg, 12.64 μmol, 19% yield) was obtained as a white solid.

LCMS: RT=2.813 min, MS cal.: 578.2, 579.2, [M+H]⁺=579.2, 580.2

¹H NMR (400 MHz, DMSO-d6) δ=7.75 (s, 1H), 7.62 (t, J=7.8 Hz, 1H), 7.56 (t, J=8.0 Hz, 1H), 7.45 (dd, J=10.4, 2.0 Hz, 1H), 7.28 (dd, J=8.0, 2.0 Hz, 1H), 7.22 (s, 1H), 6.87 (d, J=7.2 Hz, 1H), 6.66 (d, J=8.0 Hz, 1H), 5.36 (s, 2H), 4.03 (d, J=7.0 Hz, 2H), 3.90 (s, 3H), 3.81 (s, 2H), 2.93 (d, J=11.0 Hz, 2H), 2.63-2.58 (m, 1H), 2.23-2.15 (m, 2H), 1.83-1.63 (m, 4H), 1.36-1.26 (m, 1H), 0.64-0.57 (m, 2H), 0.41-0.35 (m, 2H)

### Example A97—Preparation of Compound 358. 2-((4-(6-((4-Chloro-2-fluorobenzyl)oxy)pyridin-2-yl)piperidin-1-yl)methyl)-4-ethoxy-1-methyl-1H-benzo[d]imidazole-6-carboxylic acid (Compound 358) (3)

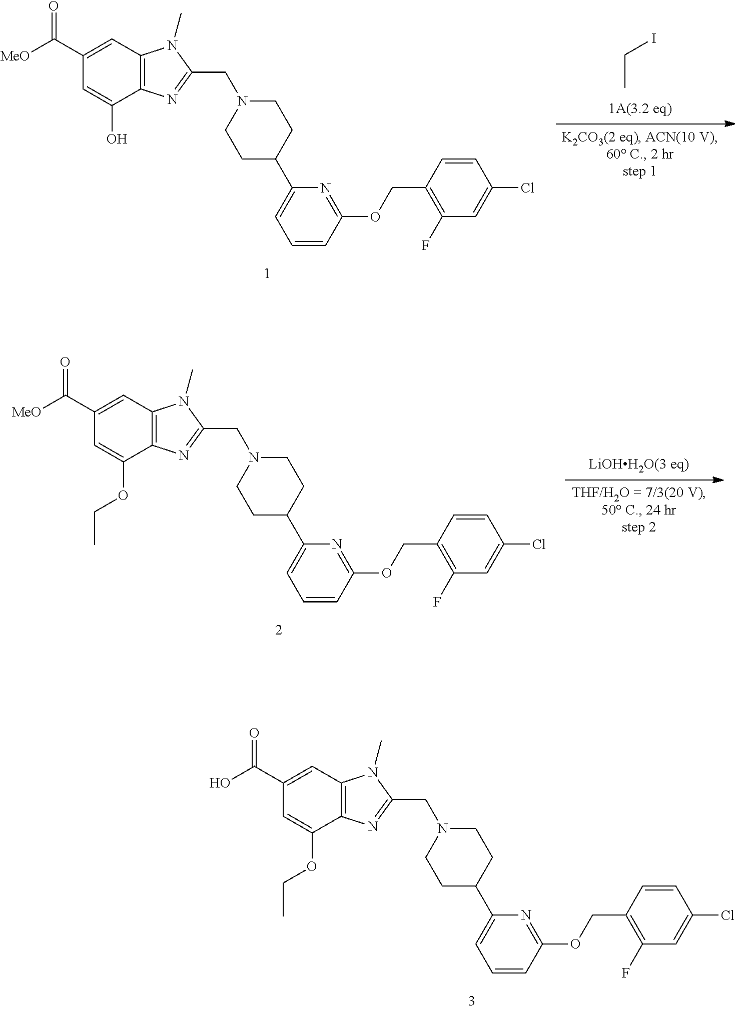

### General Procedure for Preparation of Intermediate 2:

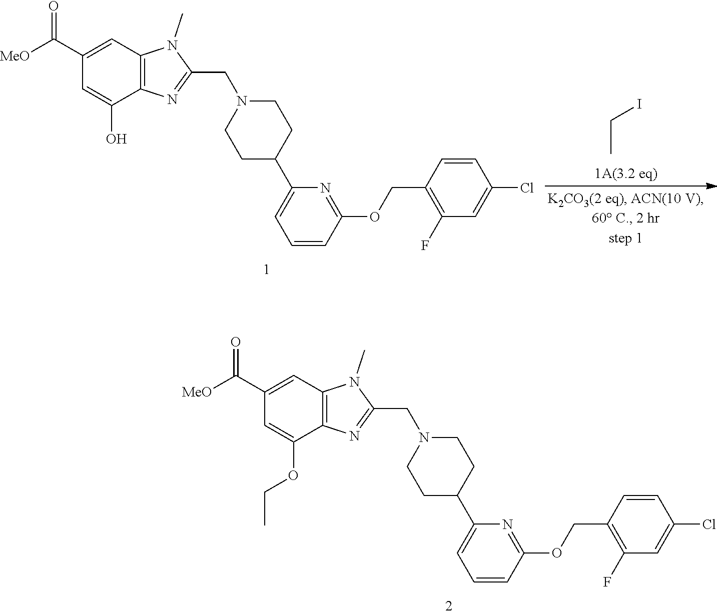

To a solution of 1 (100 mg, 185.53 μmol, 1 eq) in ACN (1 mL) was added K₂CO₃ (51.28 mg, 371.06 μmol, 2 eq) and added 1A (34.72 mg, 222.64 μmol, 17.81 μL, 3.2 eq). The mixture was stirred at 60° C. for 2 hr. LCMS (product: RT=0.488 min) showed the 1 was consumed completely. The reaction mixture was quenched by addition H₂O 5 mL at 20° C. and extracted with EtOAc 15 mL (5 mL*3). The combined organic layers were washed with brine 10 mL (5 mL*2), dried over Na₂SO₄, filtered and concentrated under reduced pressure to give a residue. The residue was purified by column chromatography (SiO₂, Petroleum ether/Ethyl acetate=1/1 to 0/1). 2 (50 mg, 88.18 μmol, 47.53% yield) was obtained as a colorless oil.

¹H NMR (400 MHz, DMSO-d6) δ=7.81 (d, J=1.2 Hz, 1H), 7.64-7.59 (m, 1H), 7.55 (t, J=8.4 Hz, 1H), 7.45 (dd, J=2.0, 10.0 Hz, 1H), 7.32-7.23 (m, 2H), 6.86 (d, J=7.6 Hz, 1H), 6.66 (d, J=8.4 Hz, 1H), 5.35 (s, 2H), 4.26 (q, J=7.2 Hz, 2H), 3.92 (s, 3H), 3.87 (s, 3H), 3.81 (s, 2H), 2.97-2.86 (m, 2H), 2.64-2.54 (m, 1H), 2.19 (br t, J=10.4 Hz, 2H), 1.82-1.63 (m, 4H), 1.42 (t, J=7.2 Hz, 3H)

### General Procedure for Preparation of Compound 358 (3):

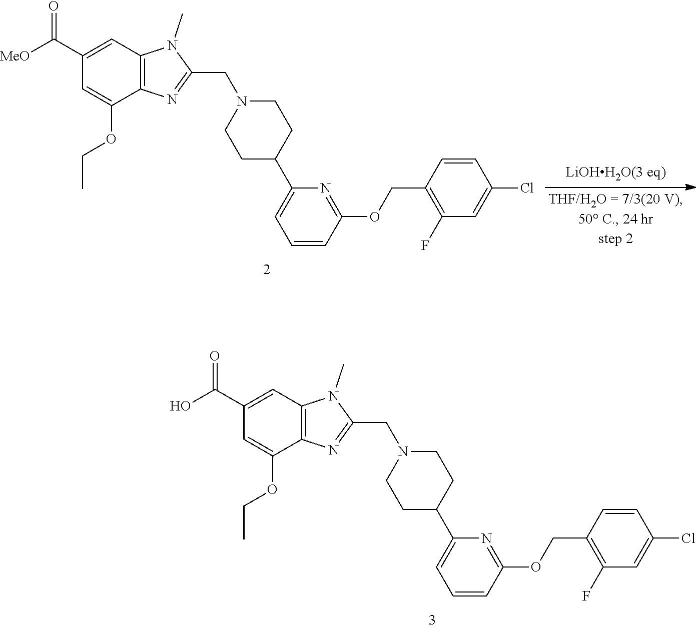 [SMILES: CCOc1cc(C(=O)OC)cc2c1nc(CN1CCC(c3cccc(OCc4ccc(Cl)cc4F)n3)CC1)n2C ; CCOc1cc(C(=O)O)cc2c1nc(CN1CCC(c3cccc(OCc4ccc(Cl)cc4F)n3)CC1)n2C]

A mixture of 2 (45 mg, 79.36 μmol, 1 eq) in THF (0.7 mL) and LiOH·H₂O (9.99 mg, 238.07 μmol, 3 eq), in H₂O (0.3 mL) was degassed and purged with N₂ for 3 times, and then the mixture was stirred at 50° C. for 24 hr under N₂ atmosphere. LCMS (ET64759-648-P1A, product: RT=1.182 min) showed the starting material was consumed completely. The reaction mixture was filtered, and the filtrate was purified by prep-HPLC (column: Waters Xbridge BEH C18 100*30 mm*10 um; mobile phase: [H₂O (10 mM NH₄HCO₃)-ACN]; gradient: 30%-60% B over 8.0 min). 2-((4-(6-((4-Chloro-2-fluorobenzyl)oxy)pyridin-2-yl)piperidin-1-yl)methyl)-4-ethoxy-1-methyl-1H-benzo[d]imidazole-6-carboxylic acid (14.65 mg, 26.49 μmol, 33.38% yield) was obtained as a white solid.

LCMS: RT=2.721 min, MS cal.: 552.2, [M+H]⁺=553.2

HPLC: RT=12.017 min

¹H NMR (400 MHz, DMSO-d6) δ=7.77 (d, J=0.8 Hz, 1H), 7.63 (t, J=7.6 Hz, 1H), 7.56 (t, J=8.4 Hz, 1H), 7.46 (dd, J=2.0, 10.0 Hz, 1H), 7.29 (dd, J=1.6, 8.4 Hz, 1H), 7.26 (s, 1H), 6.87 (d, J=7.2 Hz, 1H), 6.67 (d, J=8.0 Hz, 1H), 5.36 (s, 2H), 4.26 (q, J=7.2 Hz, 2H), 3.91 (s, 3H), 3.81 (s, 2H), 2.93 (br d, J=11.2 Hz, 2H), 2.64-2.55 (m, 1H), 2.25-2.13 (m, 2H), 1.84-1.63 (m, 4H), 1.42 (t, J=7.2 Hz, 3H).

### Example A98—Preparation of Compound 359, Compound 360, and Compound 361. 2-((4-(6-((4-Chloro-2-fluorobenzyl)oxy)pyridin-2-yl)piperidin-1-yl)methyl)-4-(1-fluoroethoxy)-1-methyl-1H-benzo[d]imidazole-6-carboxylic acid (Compound 359), (S)-2-((4-(6-((4-chloro-2-fluorobenzyl)oxy)pyridin-2-yl)piperidin-1-yl)methyl)-4-(1-fluoroethoxy)-1-methyl-1H-benzo[d]imidazole-6-carboxylic acid (Compound 360) & (R)-2-((4-(6-((4-Chloro-2-fluorobenzyl)oxy)pyridin-2-yl)piperidin-1-yl)methyl)-4-(1-fluoroethoxy)-1-methyl-1H-benzo[d]imidazole-6-carboxylic acid (Compound 361)

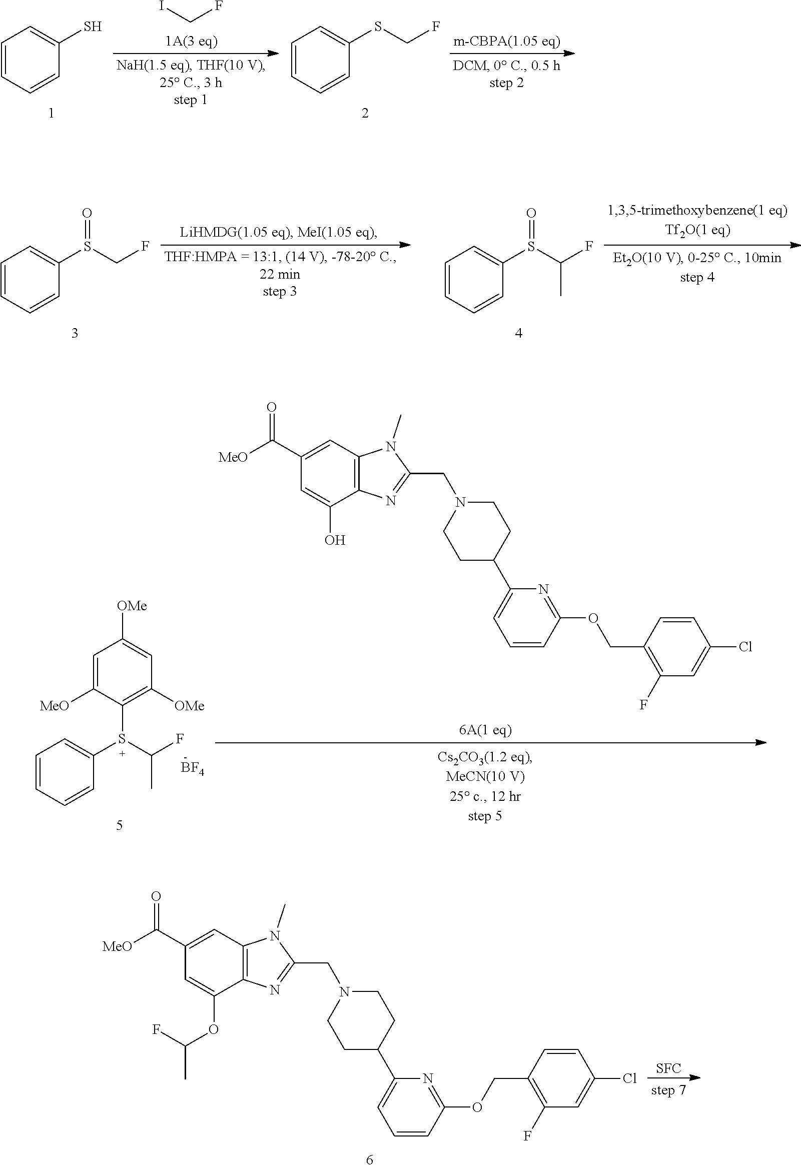

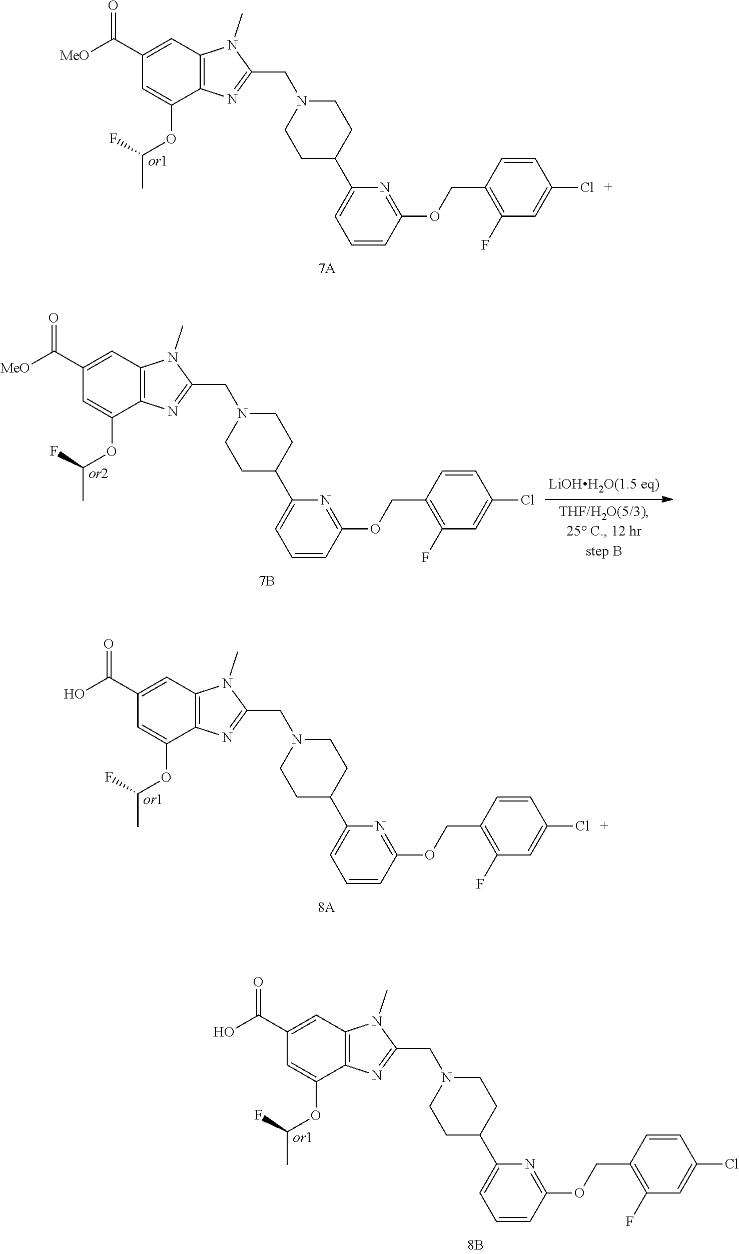

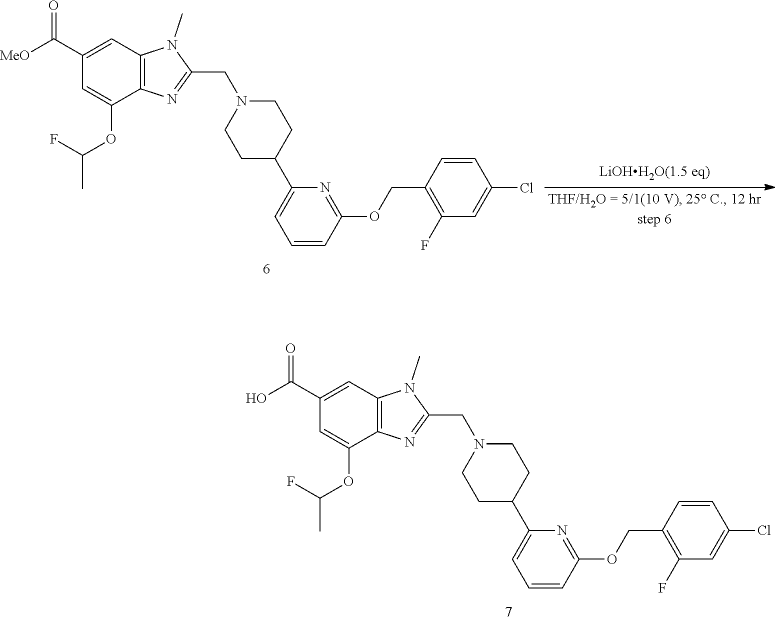 [SMILES: COC(=O)c1cc(OC(C)F)c2nc(CN3CCC(c4cccc(OCc5ccc(Cl)cc5F)n4)CC3)n(C)c2c1 ; CC(F)Oc1cc(C(=O)O)cc2c1nc(CN1CCC(c3cccc(OCc4ccc(Cl)cc4F)n3)CC1)n2C]

### General Procedure for Preparation of Intermediate 2:

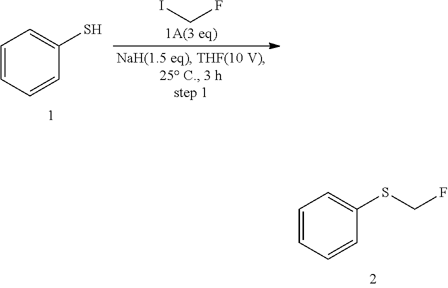

Equip a 250 mL three-necked round bottom flask, thermometer, N₂ balloon. THF (50 mL) was charged to the round bottom flask under N₂, then benzenethiol (5 g, 45.38 mmol, 4.64 mL, 1 eq) was added to the mixture at 25° C., NaH (2.72 g, 68.07 mmol, 60% purity, 1.5 eq). After 30 min, (21.77 g, 136.15 mmol, 3 eq) was added. After the addition, the mixture was stirred at 25° C. for 2.5 hr. The reaction was monitored by TLC (Petroleum ether:Ethyl acetate=1:0, R_{f}=0.51) Then to the mixture was added H₂O (20 mL) and extracted with ethyl acetate 150 mL (50 mL*3), the combined organic phase was washed with brine and dried over Na₂SO₄, the organic phase was concentrated under reduced pressure at 40° C. to give a residue. The residue was purified by column chromatography (SiO₂, Petroleum ether/Ethyl acetate=1/0 to 5/1, R_{f}=0.48). 2 (8.1 g, 28.57 mmol, 97% yield) was obtained as a yellow solid.

¹H NMR (400 MHz, CHCl3-d) δ=7.51 (d, J=7.6 Hz, 2H), 7.39-7.30 (m, 3H), 5.89-5.63 (d, J=52.8 Hz, 2H)

### General Procedure for Preparation of Intermediate 3:

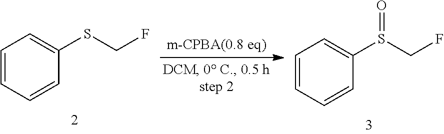 [SMILES: FCSc1ccccc1 ; O=S(CF)c1ccccc1]

Equip a 250 mL three-necked round bottom flask and thermometer, N₂ balloon. DCM (57 mL) was charged to the 250 mL flask, then 2 (5.7 g, 40.09 mmol, 1 eq) was added under N₂. m-CPBA (6.51 g, 32.07 mmol, 85% purity, 0.8 eq) was added in portions to the reaction mixture at 0° C. After the addition, the reaction mixture was stirred for 30 min at 0° C. TLC (Petroleum ether:Ethyl acetate=3:1, R_{f}=0.55) indicated the reaction was clean according to TLC. After 0.5 hr, 1M NaOH aqueous solution (30 mL) was slowly added at 0° C. The layers were then separated, and the aqueous layer was extracted with DCM (10 mL*3). The combined organic phase was washed with brine and dried over Na₂SO₄ and concentrated under vacuum to give a residue. The residue was purified by column chromatography (SiO₂, Petroleum ether/Ethyl acetate=1/0 to 3/1). 3 (4.68 g, 29.58 mmol, 74% yield) was obtained as a yellow oil.

¹H NMR (400 MHz, CHCl3-d) δ=7.72-7.67 (m, 2H), 7.61-7.57 (m, 3H), 5.17-5.03 (d, J=48.0 Hz, 2H)

### General Procedure for Preparation of Intermediate 4:

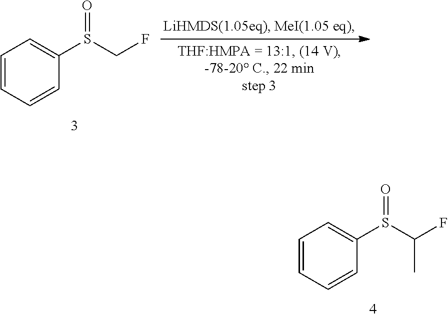

Equip a 50 mL three-necked round bottom flask, addition funnel and thermometer, N₂ balloon etc. THF (13 mL) and HMPA (1 mL) was charged to the 50 mL three-necked round bottom flask, then 3 (1 g, 6.32 mmol, 1 eq) and CH₃I (942.12 mg, 6.64 mmol, 413.21 μL, 1.05 eq) was added at 20° C. within 1 min. At −78° C. inner temperature, LiHMDS (1 M, 6.64 mL, 1.05 eq) was added to the reaction mixture at −78° C. After the addition, the mixture was stirred at −78° C. for 20 min. The reaction was monitored by TLC (Petroleum ether:Ethyl acetate=1:1, R_{f}=0.51). LCMS showed Reactant 1 was consumed and desired mass was detected. The mixture was added to the saturated NH₄Cl aqueous solution (10 ml, 0° C.) and water (8 mL). The mixture was warmed to RT and ethyl acetate (10 mL) was added. The layers were separated, and the aqueous layer was extracted with ethyl acetate 120 mL (40 mL*3). The combined organic layers were washed with water (10 mL*3), dried over Na₂SO₄, and concentrated under vacuum. The residue was purified by column chromatography (SiO₂, Petroleum ether/Ethyl acetate=100/1 to 0/1). 4 (0.49 g, 2.85 mmol, 45% yield) was obtained as a colorless oil.

### General Procedure for Preparation of Intermediate 5:

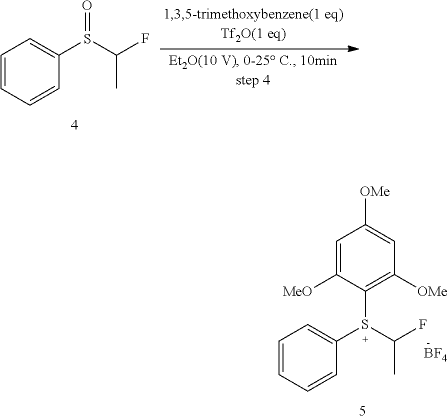

Equip a 25 mL round bottom flask. Et₂O (2 mL) was charged to the 25 mL three-necked round bottom flask, then 4 (0.2 g, 1.16 mmol, 1 eq), 1,3,5-trimethoxybenzene (195.32 mg, 1.16 mmol, 1 eq) was added to the mixture at 25° C. The resulting solution was cooled down to 0° C. and Tf₂O (327.65 mg, 1.16 mmol, 191.61 μL, 1 eq) was added dropwise. After the addition, the reaction was stirred for 10 min. The reaction was monitored by TLC (Petroleum ether:Ethyl acetate=0:1, R_{f}=0.29). After 10 min, then Et₂O was removed with a syringe. Then, Et₂O (5 mL) was added and removed with a syringe. This operation was repeated for three times. DCM (5 mL) was then added and the solution was washed with aqueous 1M NaBF₄ solution (8 mL*3). The organic layer was dried over Na₂SO₄ and concentrated under vacuum. The Compound was used directly without further purification. 5 (400 mg, crude, BF4) was obtained as a red solid.

### General Procedure for Preparation of Intermediate 6:

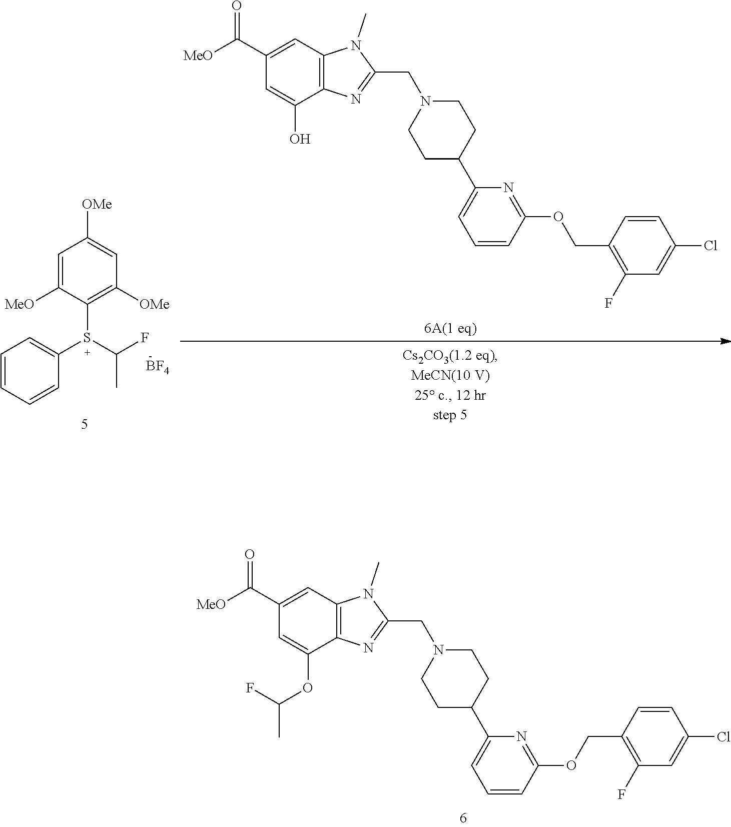

To a solution of 6A (100 mg, 185.53 μmol, 1 eq) and 5 (152.21 mg, 371.06 μmol, 2 eq, BF4-) in ACN (1 mL) was added Cs₂CO₃ (72.54 mg, 222.64 μmol, 1.2 eq). The mixture was stirred at 25° C. for 12 hr. LCMS showed 6A was consumed and desired mass was detected. The reaction mixture was partitioned between EtOAc and water. The mixture was poured into separatory funnel and separated. The aqueous layer was extracted with EtOAc (3 mL*3). The combined organic layer was washed with brine (2 mL*2), dried over Na₂SO₄, filtered and the filtrate was concentrated to give a crude product. The residue was purified by Prep-HPLC (column: Waters xbridge 150*25 mm 10 um; mobile phase: [H₂O (10 mM NH₄HCO₃)-ACN]; gradient: 50%-80% B over 8.0 min). 6 (65 mg, 111.10 μmol, 60% yield) was obtained as a white solid.

LCMS: RT=0.489 min, MS cal.: 584.2, 585.2, [M+H]⁺=585.1, 587.1

¹H NMR (400 MHz, DMSO-d6) δ=7.98 (s, 1H), 7.62 (t, J=7.8 Hz, 1H), 7.55 (t, J=8.0 Hz, 1H), 7.49 (s, 1H), 7.45 (dd, J=10.0, 1.8 Hz, 1H), 7.28 (br d, J=8.0 Hz, 1H), 6.86 (d, J=7.6 Hz, 1H), 6.73-6.53 (dq, J=63.2, 4.4 Hz, 1H), 6.67 (d, J=8.0 Hz, 1H), 5.35 (s, 2H), 3.95 (s, 3H), 3.88 (s, 3H), 3.84 (s, 2H), 2.96-2.88 (m, 2H), 2.63-2.54 (m, 1H), 2.21 (t, J=11.2 Hz, 2H), 1.82-1.75 (m, 2H), 1.75-1.63 (m, 5H)

### General Procedure for Preparation of Compound 359 (7):

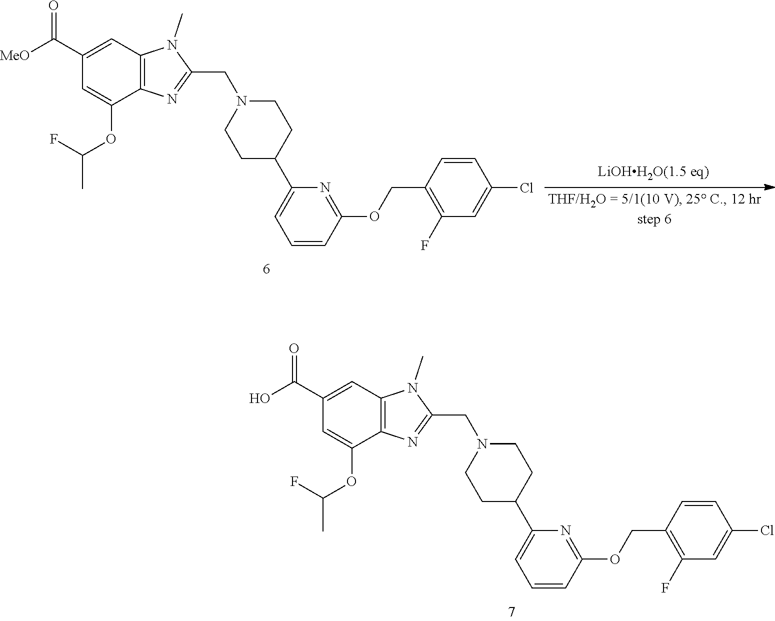 [SMILES: COC(=O)c1cc(OC(C)F)c2nc(CN3CCC(c4cccc(OCc5ccc(Cl)cc5F)n4)CC3)n(C)c2c1 ; CC(F)Oc1cc(C(=O)O)cc2c1nc(CN1CCC(c3cccc(OCc4ccc(Cl)cc4F)n3)CC1)n2C]

To a solution of 6 (60 mg, 102.56 μmol, 1 eq) in THF (0.5 mL) and H₂O (0.1 mL) was added LiOH·H₂O (6.45 mg, 153.84 μmol, 1.5 eq). The mixture was stirred at 25° C. for 12 hr. LCMS showed 6 was consumed and desired mass was detected. The was purified directly. The residue was purified by Prep-HPLC (column: Waters Xbridge BEH C18 100*30 mm*10 um; mobile phase: [H₂O (10 mM NH₄HCO₃)-ACN]; gradient: 25%-55% B over 8.0 min). 2-((4-(6-((4-chloro-2-fluorobenzyl)oxy)pyridin-2-yl)piperidin-1-yl)methyl)-4-(1-fluoroethoxy)-1-methyl-1H-benzo[d]imidazole-6-carboxylic acid (29.48 mg, 51.28 μmol, 50.00% yield, 99.32% purity) was obtained as a white solid.

LCMS: RT=2.825 min, MS cal.: 570.2, 572.2, [M+H]⁺=571.2, 573.2

HPLC: RT=12.213 purity: 99.32%

¹H NMR (400 MHz, DMSO-d6) δ=7.93 (d, J=1.2 Hz, 1H), 7.65-7.59 (m, 1H), 7.55 (t, J=8.2 Hz, 1H), 7.48 (s, 1H), 7.45 (dd, J=10.0, 2.0 Hz, 1H), 7.28 (dd, J=8.0, 2.0 Hz, 1H), 6.86 (d, J=7.6 Hz, 1H), 6.71-6.52 (dq, J=62.8, 4.8 Hz, 1H), 6.66 (d, J=8.0 Hz, 1H), 5.35 (s, 2H), 3.94 (s, 3H), 3.83 (s, 2H), 2.92 (br dd, J=7.8, 3.2 Hz, 2H), 2.63-2.54 (m, 1H), 2.20 (br t, J=11.6 Hz, 2H), 1.81-1.75 (m, 2H), 1.71 (d, J=4.8 Hz, 3H), 1.75-1.63 (m, 2H)

### General Procedure for Preparation of Intermediate 7A & 7B:

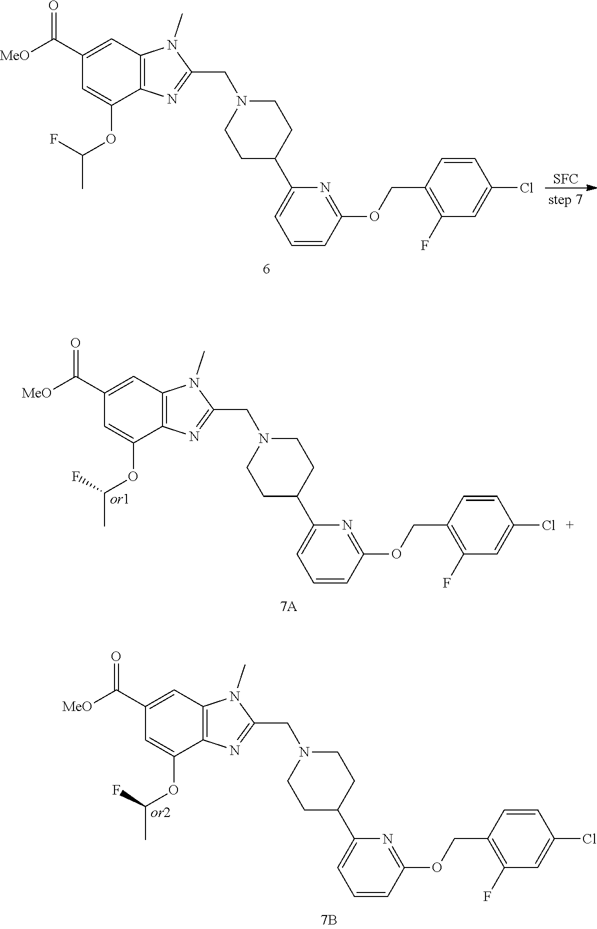

6 (280 mg, 478.60 μmol, 1 eq) was further separated by SFC (column: DAICEL CHIRALPAK AD (250 mm*30 mm, 10 um); mobile phase: [CO₂-EtOH]; B %: 40%, isocratic elution mode). The reaction mixture was concentrated under reduced pressure to remove EtOH. 7A (100 mg, 170.93 μmol, 36% yield) was obtained as a white solid. 7B (50 mg, 85.46 μmol, 18% yield) was obtained as a white solid.

LCMS: RT=1.693 min, MS cal.: 584.2, 585.2, [M+H]⁺=585.1, 587.0

LCMS: RT=1.722 min, MS cal.: 584.2, 585.2, [M+H]⁺=585.1, 587.0

SFC: ee %=100.00%

SFC: ee %=98.22%

¹H NMR (400 MHz, DMSO-d6) δ=7.98 (s, 1H), 7.62 (t, J=7.8 Hz, 1H), 7.55 (t, J=8.0 Hz, 1H), 7.49 (s, 1H), 7.45 (dd, J=10.0, 2.0 Hz, 1H), 7.28 (dd, J=8.0, 2.0 Hz, 1H), 6.86 (d, J=7.2 Hz, 1H), 6.72-6.54 (dq, J=62.8, 4.4 Hz, 1H), 6.66 (d, J=8.0 Hz, 1H), 5.35 (s, 2H), 3.95 (s, 3H), 3.88 (s, 3H), 3.84 (s, 2H), 2.92 (br d, J=8.8 Hz, 2H), 2.61-2.56 (m, 1H), 2.20 (br t, J=11.6 Hz, 2H), 1.83-1.76 (m, 2H), 1.74-1.64 (m, 5H)

¹H NMR (400 MHz, DMSO-d6) δ=7.98 (d, J=1.4 Hz, 1H), 7.65-7.60 (m, 1H), 7.56 (t, J=8.2 Hz, 1H), 7.49 (s, 1H), 7.46 (dd, J=10.0, 2.0 Hz, 1H), 7.29 (dd, J=8.2, 1.8 Hz, 1H), 6.87 (d, J=7.2 Hz, 1H), 6.74-6.54 (dq, J=60.0, 4.8 Hz, 1H), 6.67 (d, J=8.2 Hz, 1H), 5.36 (s, 2H), 3.96 (s, 3H), 3.89 (s, 3H), 3.85 (s, 2H), 2.97-2.89 (m, 2H), 2.65-2.55 (m, 1H), 2.21 (t, J=11.6 Hz, 2H), 1.82-1.65 (m, 7H)

### General Procedure for Preparation of Compound 360 (8A):

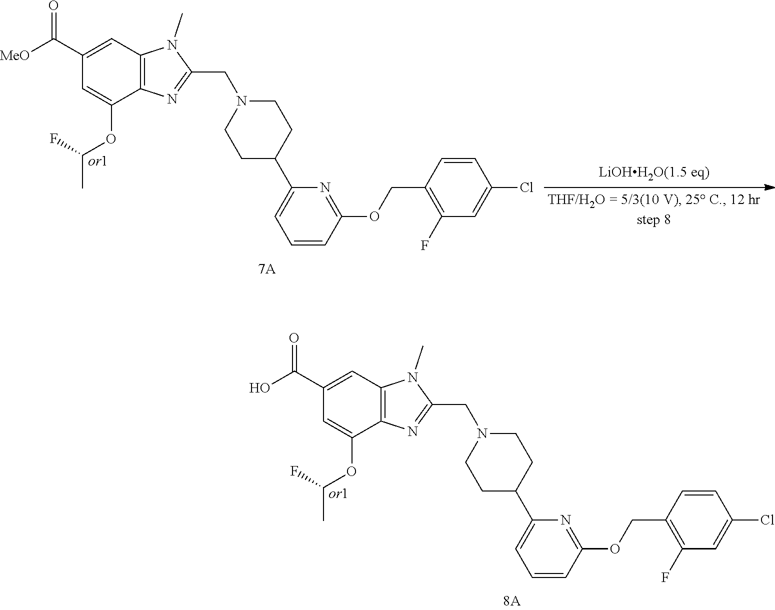 [SMILES: COC(=O)c1cc(O[C@H](C)F)c2nc(CN3CCC(c4cccc(OCc5ccc(Cl)cc5F)n4)CC3)n(C)c2c1 ; C[C@H](F)Oc1cc(C(=O)O)cc2c1nc(CN1CCC(c3cccc(OCc4ccc(Cl)cc4F)n3)CC1)n2C]

To a solution of 7A (80.00 mg, 136.74 μmol, 1 eq) in THF (0.5 mL) and H₂O (0.3 mL) was added LiOH·H₂O (8.61 mg, 205.11 μmol, 1.5 eq). The mixture was stirred at 25° C. for 12 hr.

LCMS showed 7A was consumed and desired mass was detected. The reaction was used directly for purification. The residue was purified by Prep-HPLC (column: Waters Xbridge Prep OBD C18 150*40 mm*10 um; mobile phase: [H₂O (10 mM NH₄HCO₃)-ACN]; gradient: 25%-55% B over 8.0 min). (S)-2-((4-(6-((4-chloro-2-fluorobenzyl)oxy)pyridin-2-yl)piperidin-1-yl)methyl)-4-(1-fluoroethoxy)-1-methyl-1H-benzo[d]imidazole-6-carboxylic acid (29.45 mg, 51.10 μmol, 37.37% yield, 99.07% purity) was obtained as a white solid.

LCMS: RT=1.230 min, MS cal.: 570.18/571.02, [M+H]⁺=571.1/572.1

SFC: ee %=100.00%

HPLC: RT=12.068 min, purity: 99.07%

¹H NMR (400 MHz, DMSO-d6) δ=7.93 (s, 1H), 7.62 (t, J=7.6 Hz, 1H), 7.55 (t, J=8.2 Hz, 1H), 7.48 (s, 1H), 7.45 (dd, J=10.0, 2.0 Hz, 1H), 7.28 (dd, J=8.2, 1.8 Hz, 1H), 6.86 (d, J=7.4 Hz, 1H), 6.71-6.52 (dq, J=62.8, 4.8 Hz, 1H), 6.66 (d, J=8.0 Hz, 1H), 5.36 (s, 2H), 3.94 (s, 3H), 3.88-3.79 (m, 2H), 2.92 (br d, J=8.6 Hz, 2H), 2.63-2.55 (m, 1H), 2.20 (br t, J=11.6 Hz, 2H), 1.77 (br t, J=11.0 Hz, 1H), 1.83-1.60 (m, 6H)

### General Procedure for Preparation of Compound 361 (8B):

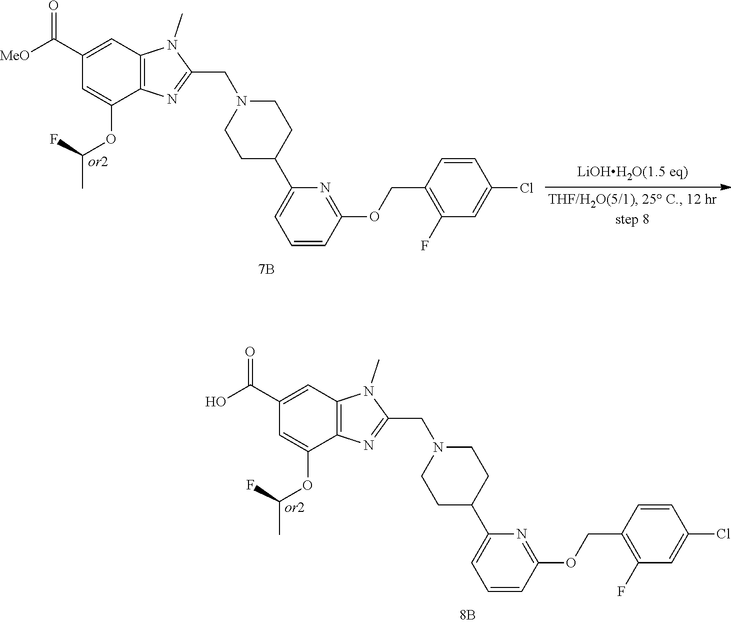 [SMILES: COC(=O)c1cc(O[C@@H](C)F)c2nc(CN3CCC(c4cccc(OCc5ccc(Cl)cc5F)n4)CC3)n(C)c2c1 ; C[C@@H](F)Oc1cc(C(=O)O)cc2c1nc(CN1CCC(c3cccc(OCc4ccc(Cl)cc4F)n3)CC1)n2C]

To a solution of 7B (45 mg, 76.92 μmol, 1 eq) in THF (0.5 mL) was added LiOH·H₂O (4.84 mg, 115.38 μmol, 1.5 eq) in H₂O (0.1 mL). The mixture was stirred at 25° C. for 18 hr. LCMS showed 7B was consumed and desired mass was detected. The reaction was used directly for purification. The residue was purified by Prep-HPLC (column: Waters Xbridge Prep OBD C18 150*40 mm*10 um; mobile phase: [H₂O (10 mM NH₄HCO₃)-ACN]; gradient: 25%-60% B over 8.0 min). (R)-2-((4-(6-((4-chloro-2-fluorobenzyl)oxy)pyridin-2-yl)piperidin-1-yl)methyl)-4-(1-fluoroethoxy)-1-methyl-1H-benzo[d]imidazole-6-carboxylic acid (22.38 mg, 39.19 μmol, 50.95% yield) was obtained as a white solid.

LCMS: RT=2.824 min, MS cal.: 570.18/571.02, [M+H]⁺=571.1/572.1

SFC: ee %=98.48%

HPLC: RT=12.021 min, purity: 97.92%

¹H NMR (400 MHz, DMSO-d6) δ=7.92 (s, 1H), 7.65-7.59 (m, 1H), 7.55 (t, J=8.2 Hz, 1H), 7.48 (s, 1H), 7.45 (dd, J=10.0, 2.0 Hz, 1H), 7.28 (dd, J=8.4, 2.0 Hz, 1H), 6.87 (d, J=7.2 Hz, 1H), 6.72-6.59 (dq, J=64.0, 4.0 Hz, 1H), 6.66 (d, J=8.2 Hz, 1H), 5.36 (s, 2H), 3.93 (s, 3H), 3.88-3.79 (m, 2H), 2.96-2.88 (m, 2H), 2.63-2.55 (m, 1H), 2.20 (t, J=11.6 Hz, 2H), 1.83-1.63 (m, 7H)

### Example A99—Preparation of Compound 362. 2-((3-(6-((4-Chloro-2-fluorophenoxy)methyl)pyridin-2-yl) pyrrolidin-1-yl)methyl)-4-(difluoromethoxy)-1-methyl-1H-benzo[d]imidazole-6-carboxylic acid (Compound 362) (7)

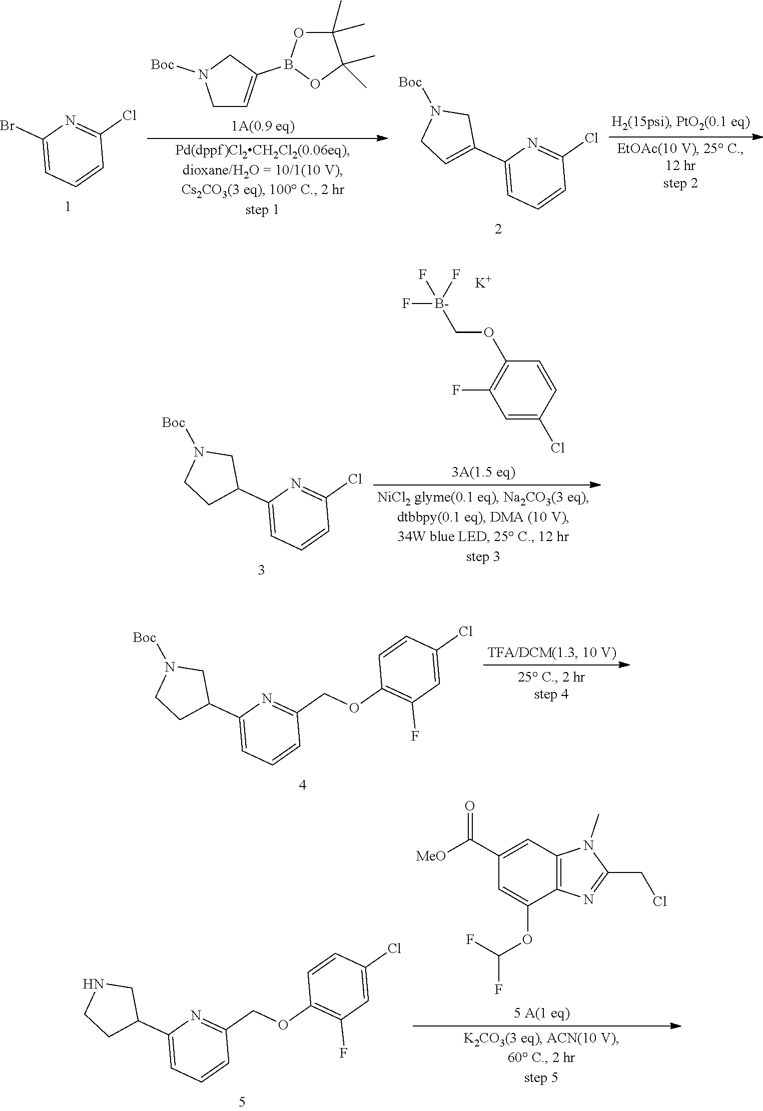

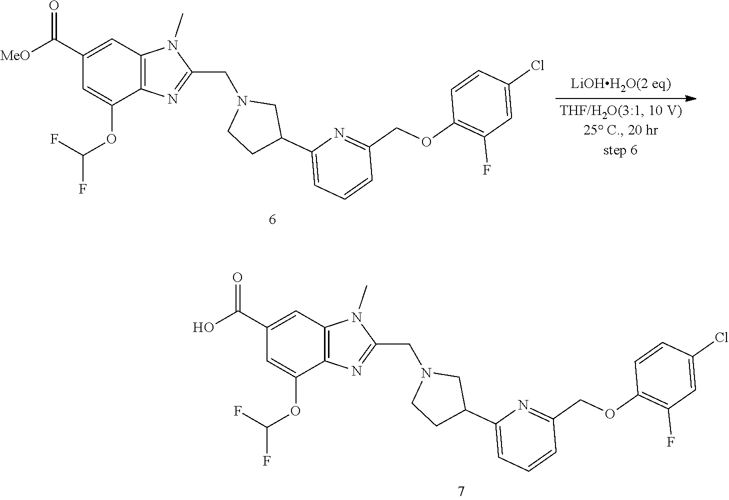 [SMILES: COC(=O)c1cc(OC(F)F)c2nc(CN3CCC(c4cccc(COc5ccc(Cl)cc5F)n4)C3)n(C)c2c1 ; Cn1c(CN2CCC(c3cccc(COc4ccc(Cl)cc4F)n3)C2)nc2c(OC(F)F)cc(C(=O)O)cc21]

### General Procedure for Preparation of Intermediate 2:

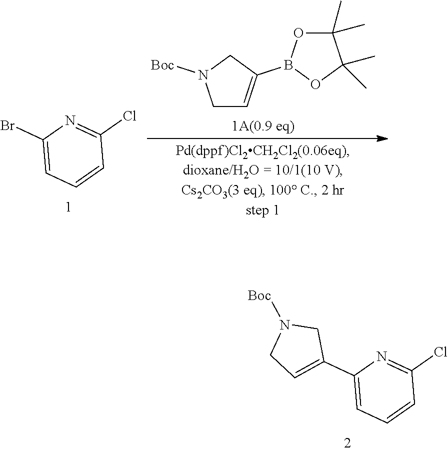 [SMILES: CC(C)(C)OC(=O)N1CC=C(B2OC(C)(C)C(C)(C)O2)C1 ; Clc1cccc(Br)n1 ; CC(C)(C)OC(=O)N1CC=C(c2cccc(Cl)n2)C1]

To a solution of 1 (6 g, 31.18 mmol, 1 eq) and 1A (8.28 g, 28.06 mmol, 0.9 eq) in dioxane (55 mL) and H₂O (5 mL) was added Cs₂CO₃ (30.48 g, 93.54 mmol, 3 eq) and Pd(dppf)Cl₂·CH₂Cl₂ (1.53 g, 1.87 mmol, 0.06 eq) at 25° C. The mixture was stirred at 100° C. for 2 hr. TLC showed 1 was consumed completely. The reaction mixture was filtered. The mixture was diluted with H₂O 70 mL and extracted with EtOAc 60 mL (20 mL×3). The combined organic layers were washed with brine 50 mL, dried over Na₂SO₄, filtered and concentrated under reduced pressure to give a residue. The residue was purified by column chromatography (SiO₂, Petroleum ether/Ethyl acetate=1/0 to 0/1). 2 (5 g, 17.81 mmol, 57.12% yield) was obtained as a yellow solid.

¹H NMR (400 MHz, CHCl3-d) δ=7.90-7.83 (m, 1H), 7.73-7.60 (m, 1H), 7.42 (d, J=8.0 Hz, 1H), 6.74 (d, J=16.8 Hz, 1H), 4.40 (br s, 2H), 4.26 (br s, 2H), 1.45 (d, J=6.8 Hz, 9H)

### General Procedure for Preparation of Intermediate 3:

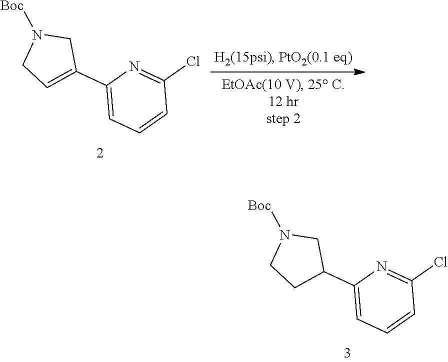 [SMILES: CC(C)(C)OC(=O)N1CC=C(c2cccc(Cl)n2)C1 ; CC(C)(C)OC(=O)N1CCC(c2cccc(Cl)n2)C1]

A mixture of Compound 2 (5 g, 17.81 mmol, 1 eq) and PtO₂ (404.41 mg,) in EtOAc (50 mL) was stirred under H₂ (15 psi) at 60° C. for 12 hour. TLC showed 2 was consumed completely. The reaction mixture was diluted with EtOAc (20 mL), and then filtered through celite pad. The filter cake was rinsed with EtOAc (5 ml×3), and the filtrate was concentrated. 3 (3.6 g, 12.73 mmol, 71.49% yield) was obtained as a colorless oil.

¹H NMR (400 MHz, CHCl3-d) δ=7.52 (t, J=7.6 Hz, 1H), 7.13 (d, J=8.0 Hz, 1H), 7.04 (d, J=7.6 Hz, 1H), 3.79-3.68 (m, 1H), 3.56 (d, J=8.4 Hz, 1H), 3.61-3.28 (m, 3H), 2.24-2.05 (m, 2H), 1.40 (s, 9H)

### General Procedure for Preparation of Intermediate 4:

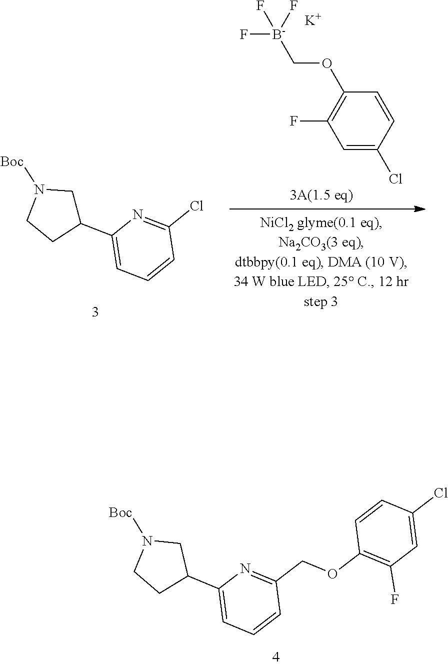 [SMILES: CC(C)(C)OC(=O)N1CCC(c2cccc(Cl)n2)C1 ; Fc1cc(Cl)ccc1OC[B-](F)(F)F ; [K+] ; CC(C)(C)OC(=O)N1CCC(c2cccc(COc3ccc(Cl)cc3F)n2)C1]

To a solution of 3 (300 mg, 1.06 mmol, 1 eq) in DMA (3 mL) was added 3A (424.07 mg, 1.59 mmol, 1.5 eq, K⁺), NiCl₂ glyme (23.31 mg, 106.10 μmol, 0.1 eq) and Na₂CO₃ (337.35 mg, 3.18 mmol, 3 eq) dtbbpy (28.48 mg, 106.10 μmol, 0.1 eq), (IR(dF(CF₃)ppy)₂(bpy))PF₆ (53.56 mg, 53.05 μmol, 0.05 eq). The mixture was stirred at 25° C. for 12 hr. TLC showed 3 was consumed completely. The reaction mixture was diluted with H₂O 8 mL and extracted with EtOAc 6 mL. The combined organic layers were washed with brine 5 mL, dried over Na₂SO₄, filtered, and concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 60%-95% B over 8.0 min). 4 (40 mg, 98.31 μmol, 4.63% yield) as a yellow oil.

LCMS: RT=1.613 min, MS cal.: 406.15, [M+H]⁺=407.0

¹H NMR (400 MHz, CHCl3-d) δ=7.81 (t, J=7.7 Hz, 1H), 7.47 (dd, J=2.4, 11.1 Hz, 1H), 7.38 (d, J=7.7 Hz, 1H), 7.32 (d, J=7.7 Hz, 1H), 7.34-7.26 (m, 1H), 7.30-7.26 (m, 1H), 7.23-7.18 (m, 1H), 5.24 (s, 2H), 3.71-3.64 (m, 1H), 3.54 (d, J=8.0 Hz, 1H), 3.51-3.43 (m, 1H), 3.42-3.34 (m, 2H), 2.26-2.13 (m, 1H), 2.11-1.97 (m, 1H), 1.41 (d, J=2.7 Hz, 9H)

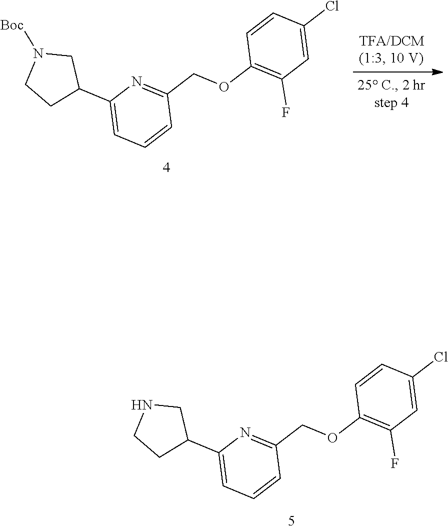 [SMILES: CC(C)(C)OC(=O)N1CCC(c2cccc(COc3ccc(Cl)cc3F)n2)C1 ; Fc1cc(Cl)ccc1OCc1cccc(C2CCNC2)n1]

To a solution of 4 (40 mg, 98.31 μmol, 1 eq) in TFA (0.1 mL) was added DCM (0.3 mL) at 25° C. The mixture was stirred at 25° C. for 2 hr. TLC showed 4 was consumed completely. The reaction mixture was adjusted to pH 6 with NaHCO₃. The aqueous layer was extracted with EtOAc (2 mL×3). The combined organic phase was washed with brine (5 mL×2), dried with anhydrous Na₂SO₄, filtered and concentrated in vacuum. 5 (25 mg, 81.50 μmol, 82.90% yield) was obtained as a yellow oil.

LCMS: RT-0.396 min, MS cal.: 306.09, [M+H]⁺=307.1

¹H NMR (400 MHz, CHCl3-d) δ=7.77 (t, J=7.6 Hz, 1H), 7.44-7.38 (m, 1H), 7.35 (d, J=7.6 Hz, 1H), 7.29 (d, J=8.0 Hz, 1H), 7.21 (d, J=8.8 Hz, 1H), 7.17-7.12 (m, 1H), 5.18 (s, 2H), 3.52 (d, J=7.6 Hz, 1H), 3.42 (dd, J=8.0, 11.2 Hz, 1H), 3.22-3.10 (m, 3H), 2.25-2.17 (m, 1H), 1.97-1.86 (m, 1H)

### General Procedure for Preparation of Intermediate 6:

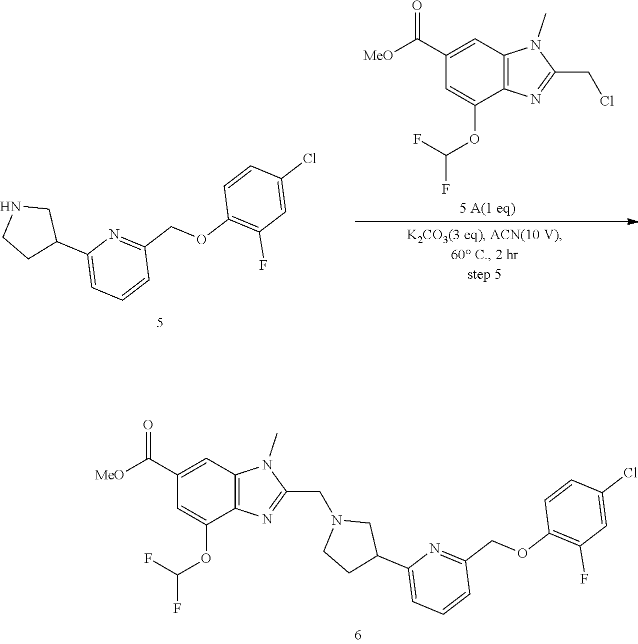 [SMILES: Fc1cc(Cl)ccc1OCc1cccc(C2CCNC2)n1 ; COC(=O)c1cc(OC(F)F)c2nc(CCl)n(C)c2c1 ; COC(=O)c1cc(OC(F)F)c2nc(CN3CCC(c4cccc(COc5ccc(Cl)cc5F)n4)C3)n(C)c2c1]

To a solution of 5 (30 mg, 97.80 μmol, 1 eq) and 5A (29.80 mg, 97.80 μmol, 1 eq) in ACN (0.5 mL) was added K₂CO₃ (27.03 mg, 195.59 μmol, 2 eq). The mixture was stirred at 60° C. for 1 hr. TLC showed 5 was consumed completely. The combined crude solution was diluted with H₂O 10 mL and extracted with EtOAc 9 mL. The combined organic layers were washed with brine 10 mL, dried over Na₂SO₄, filtered, and concentrated under reduced pressure to give a residue. Compound 6 (45 mg, 78.26 μmol, 82.5% total yield) as a yellow oil.

### General Procedure for Preparation of Compound 362 (7):

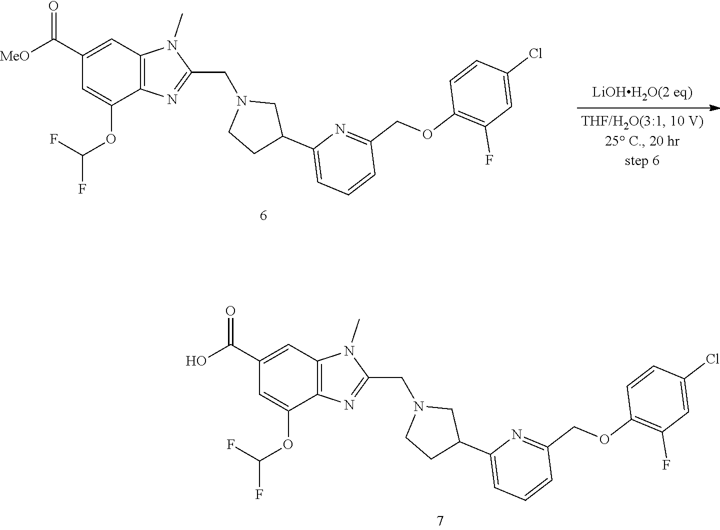 [SMILES: COC(=O)c1cc(OC(F)F)c2nc(CN3CCC(c4cccc(COc5ccc(Cl)cc5F)n4)C3)n(C)c2c1 ; Cn1c(CN2CCC(c3cccc(COc4ccc(Cl)cc4F)n3)C2)nc2c(OC(F)F)cc(C(=O)O)cc21]

To a solution of 6 (45 mg, 78.26 μmol, 1 eq) in THF (0.3 mL) was added LiOH·H₂O (4.38 mg, 104.35 μmol, 1.5 eq), LiOH·H₂O (6.57 mg, 156.53 μmol, 2 eq) and H₂O (0.1 mL). The mixture was stirred at 25° C. for 20 hr. TLC showed 6 was consumed completely. The reaction mixture was filtered to give a solution. The combined crude solution was purified by prep-HPLC (column: Waters xbridge 150*25 mm 10 um; mobile phase: [H₂O (10 mM NH₄HCO₃)-ACN]; gradient: 30%-65% B over 8.0 min). 2-((3-(6-((4-Chloro-2-fluorophenoxy)methyl)pyridin-2-yl) pyrrolidin-1-yl)methyl)-4-(difluoromethoxy)-1-methyl-1H-benzo[d]imidazole-6-carboxylic acid (29.9 mg, 26.74 μmol, 32.26% total yield) was obtained as a white solid.

LCMS: RT=2.604 min, MS cal.: 560.14, [M+H]⁺=561.2.

¹H NMR (400 MHz, CHCl3-d) δ=8.06 (d, J=1.2 Hz, 1H), 7.76 (t, J=7.6 Hz, 1H), 7.64 (t, J=76 Hz, 1H), 7.51 (s, 1H), 7.49-7.43 (m, 1H), 7.30-7.25 (m, 1H), 7.35-7.25 (m, 2H), 7.21-7.16 (m, 1H), 5.23 (s, 2H), 4.00 (s, 2H), 3.94 (s, 3H), 3.56-3.46 (m, 1H), 3.03 (t, J=8.4 Hz, 1H), 2.85-2.67 (m, 3H), 2.29-2.16 (m, 1H), 2.08-1.93 (m, 1H)

### Example A100—Preparation of Compound 307. 2-((4-(6-((4-Chloro-2-fluorobenzyl)oxy)pyridin-2-yl)piperidin-1-yl)methyl)-4-hydroxy-1-methyl-1H-benzo[d]imidazole-6-carboxylic acid (Compound 307) (2)

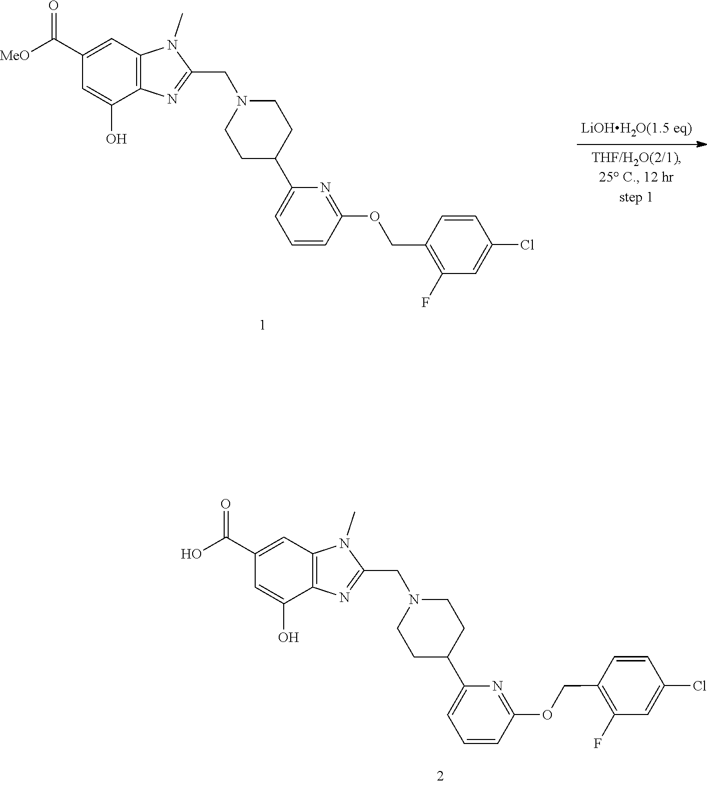 [SMILES: COC(=O)c1cc(O)c2nc(CN3CCC(c4cccc(OCc5ccc(Cl)cc5F)n4)CC3)n(C)c2c1 ; Cn1c(CN2CCC(c3cccc(OCc4ccc(Cl)cc4F)n3)CC2)nc2c(O)cc(C(=O)O)cc21]

### General Procedure for Preparation of Compound 307 (2):

To a solution of 1 (50 mg, 92.76 μmol, 1 eq) in THF (0.4 mL) and H₂O (0.2 mL) was added LiOH·H₂O (5.84 mg, 139.15 μmol, 1.5 eq). The mixture was stirred at 25° C. for 12 hr. LCMS showed 1 was consumed completely and one main peak with desired mass was detected. The reaction mixture was diluted with H₂O (10 mL) and extracted with EtOAc (10 mL*3). The combined organic layers were washed with brine (10 mL*3), dried over Na₂SO₄, filtered, and concentrated under reduced pressure to give a residue. The residue was purified by prep-HPLC (column: Waters Xbridge Prep OBD C18 150* 40 mm*10 um; mobile phase: [H₂O (10 mM NH₄HCO₃)-ACN]; gradient: 15%-65% B over 8.0 min). 2-((4-(6-((4-Chloro-2-fluorobenzyl)oxy)pyridin-2-yl)piperidin-1-yl)methyl)-4-hydroxy-1-methyl-1H-benzo[d]imidazole-6-carboxylic acid (27.5 mg, 52.38 μmol, 56.47% yield) was obtained as a white solid.

LCMS: RT=2.668 min, MS cal.: 524.2, [M+H]⁺=525.2

HPLC: RT=10.989 min, purity: 100%

¹H NMR (400 MHz, MeOH-d4) δ=7.71 (s, 1H), 7.60-7.54 (m, 1H), 7.52-7.45 (m, 1H), 7.34 (s, 1H), 7.19 (s, 1H), 6.85-6.79 (m, 1H), 6.65-6.60 (m, 1H), 5.40 (s, 2H), 3.96 (s, 3H), 3.92 (s, 2H), 3.11-3.03 (m, 2H), 2.71-2.60 (m, 1H), 2.42-2.30 (m, 2H), 1.88 (br d, J=3.2 Hz, 4H)

### Example A101—Preparation of Compound 363. 2-(((1r,4r)-4-(6-((4-Chloro-2-fluorobenzyl)oxy)pyridin-2-yl)cyclohexyl)(methyl)amino)-4-methoxy-1-methyl-1H-benzo[d]imidazole-6-carboxylic acid (Compound 363) (5)

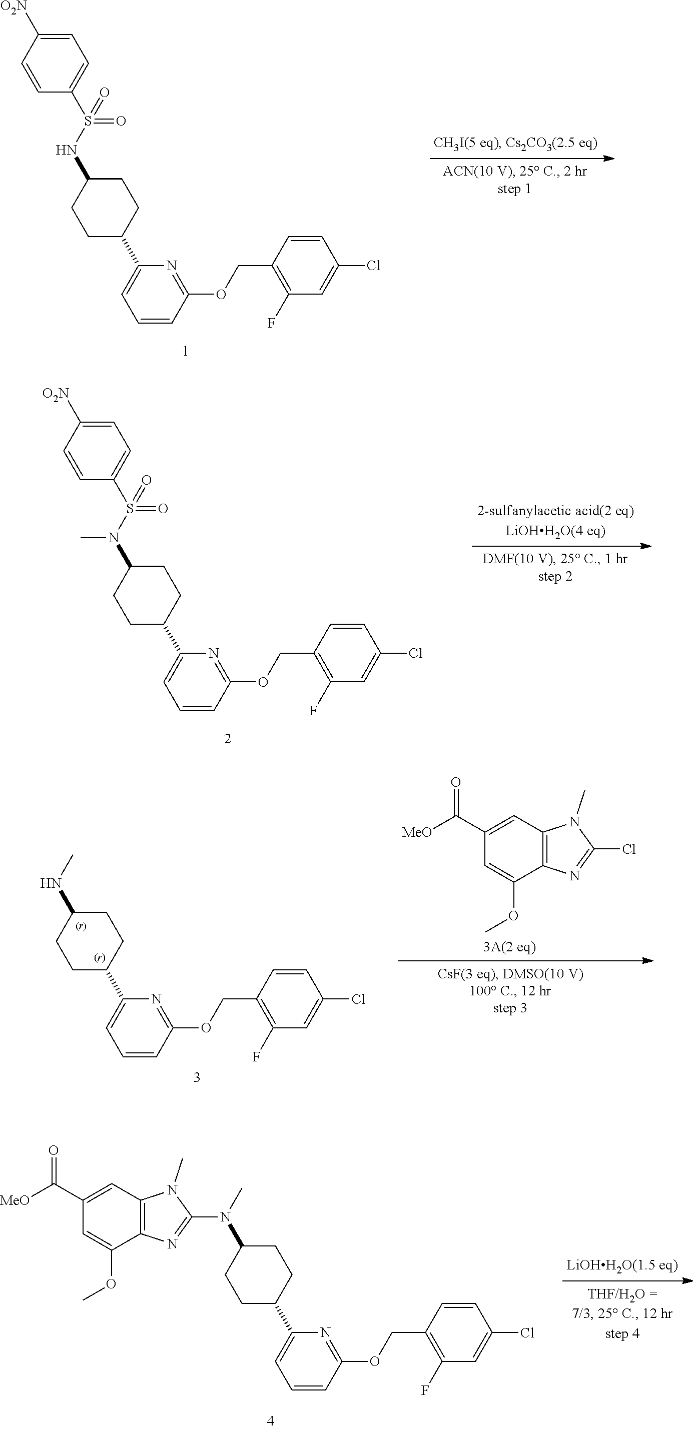

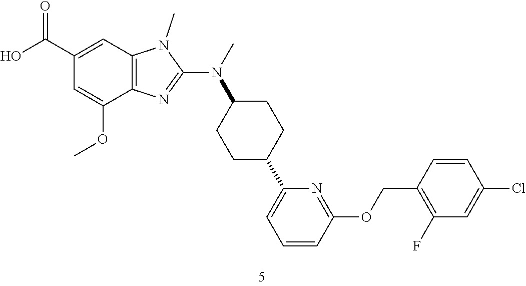 [SMILES: COc1cc(C(=O)O)cc2c1nc(N(C)[C@H]1CC[C@H](c3cccc(OCc4ccc(Cl)cc4F)n3)CC1)n2C]

### General Procedure for Preparation of Intermediate 2:

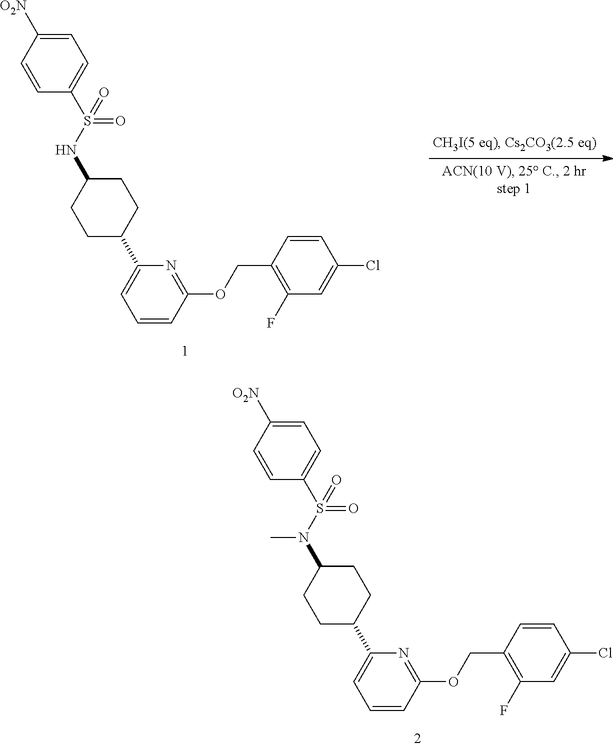 [SMILES: O=[N+]([O-])c1ccc(S(=O)(=O)N[C@H]2CC[C@H](c3cccc(OCc4ccc(Cl)cc4F)n3)CC2)cc1 ; CN([C@H]1CC[C@H](c2cccc(OCc3ccc(Cl)cc3F)n2)CC1)S(=O)(=O)c1ccc([N+](=O)[O-])cc1]

ACN (25 mL) was charged to the 50 mL three-necked round bottom flask, 1 (2.5 g, 4.81 mmol, 1 eq) and Cs₂CO₃ (3.92 g, 12.02 mmol, 2.5 eq) was added to the mixture at 25° C. At 25° C., CH₃I (3.41 g, 24.04 mmol, 1.50 mL, 5 eq) was added to the reaction mixture. After the addition, the mixture was stirred at 25° C. for 2 hr. LCMS showed 1 was consumed completely and one main peak with desired mass was detected. The reaction mixture was added to H₂O (50 mL) at 25° C. and extracted with EtOAc (100 mL*3). Then organic phase was washed by brine (50 mL*2). The organic phase was dried over Na₂SO₄, filtered. The organic phase was concentrated under reduced pressure at 40° C. to give a residue. 2 (2.5 g, 4.68 mmol, 97.37% yield) was obtained as a yellow solid.

LCMS: RT=0.702 min, MS cal.: 533.1, [M+H]⁺=534.2

¹H NMR (400 MHz, CHCl3-d) δ=8.41-8.36 (m, 2H), 8.07-8.02 (m, 2H), 7.49 (dd, J=7.2, 8.0 Hz, 1H), 7.44 (t, J=8.0 Hz, 1H), 7.14 (d, J=8.4 Hz, 2H), 6.68 (d, J=7.2 Hz, 1H), 6.61 (d, J=8.0 Hz, 1H), 5.40 (s, 2H), 3.95 (tt, J=4.0, 11.2 Hz, 1H), 2.86 (s, 3H), 2.47 (tt, J=3.6, 11.6 Hz, 1H), 2.00-1.91 (m, 2H), 1.74-1.57 (m, 6H)

### General Procedure for Preparation of Intermediate 3:

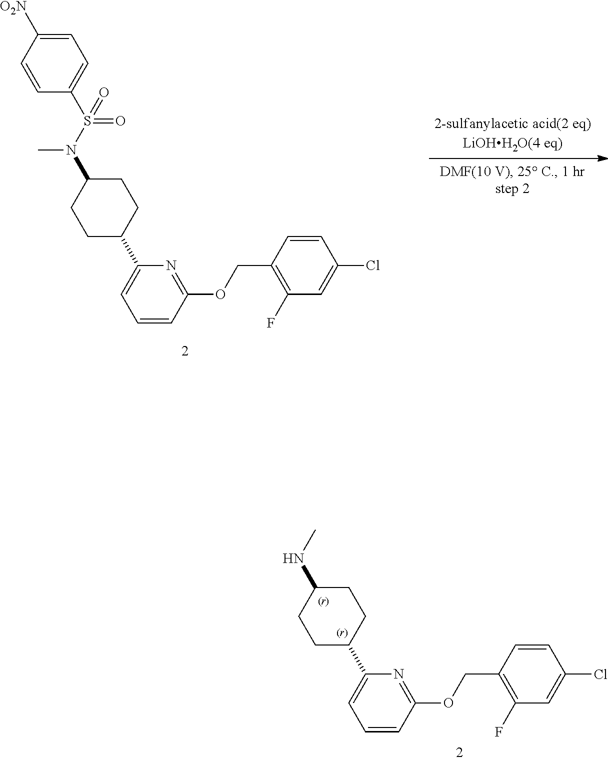

DMF (25 mL) was charged to the 50 mL three-necked round bottom flask, 2 (2.5 g, 4.68 mmol, 1 eq) and LiOH·H₂O (785.84 mg, 18.73 mmol, 4 eq) was added at 25° C. At 25° C., 2-sulfanylacetic acid (862.52 mg, 9.36 mmol, 638.90 μL, 2 eq) was added to the reaction mixture at 25° C. After the addition, the mixture was stirred at 25° C. for 1 hr. LCMS showed 2 was consumed completely and one main peak with desired mass was detected. The reaction mixture was added to H₂O (20 mL) and extracted with EtOAc (20 mL*3). Then organic phase was washed with brine (20 mL). The organic were dried over Na₂SO₄, filtered. The organic phase was concentrated under reduced pressure at 45° C. to give a residue. The residue was purified by prep-HPLC (column: Waters Xbridge BEH C18 250*50 mm*10 um; mobile phase: [H₂O (10 mM NH₄HCO₃)-ACN]; gradient: 30%-70% B over 10.0 min). 3 (1 g, 2.87 mmol, 61.23% yield) was obtained as yellow oil.

LCMS: RT=1.247 min, MS cal.: 348.1, [M+H]⁺=349

¹H NMR (400 MHz, CHCl3-d) δ=7.53-7.40 (m, 2H), 7.16-7.09 (m, 2H), 6.72 (d, J=7.2 Hz, 1H), 6.60 (d, J=8.0 Hz, 1H), 5.41 (s, 2H), 2.68-2.45 (m, 5H), 2.15 (br d, J=11.6 Hz, 2H), 2.06-1.93 (m, 2H), 1.70-1.55 (m, 2H), 1.43-1.28 (m, 2H)

### General Procedure for Preparation of Intermediate 4:

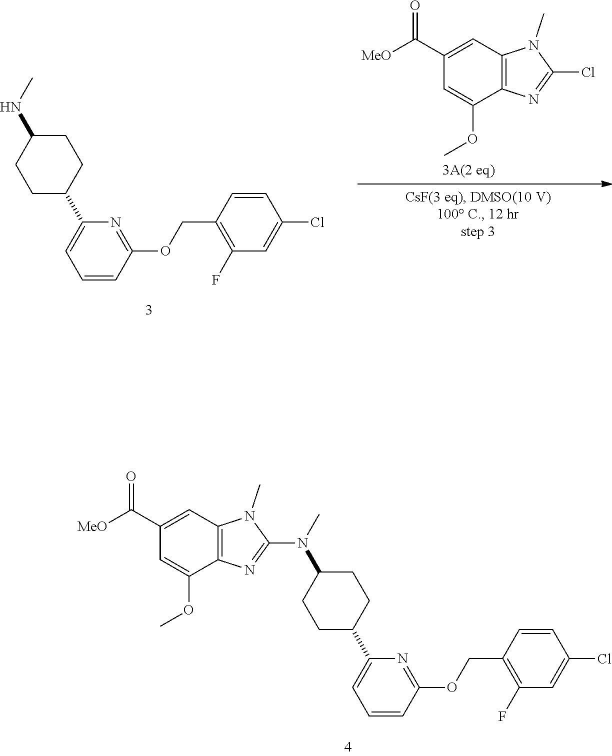

To a solution of 3A (Synthesized from Int 4) (146.01 mg, 573.33 μmol, 2 eq), 3 (100 mg, 286.66 μmol, 1 eq) in DMSO (2 mL) was added CsF (130.64 mg, 859.99 μmol, 3 eq) and molecular sieves (150 mg). The mixture was stirred at 100° C. for 12 hr. LC-MS showed 3 was consumed completely and desired mass was detected. The reaction mixture was filtered through celite pad. The filtrate was diluted with H₂O (10 mL) and extracted with EtOAc (15 mL). The combined organic layers were washed with brine (10 mL), dried over Na₂SO₄, filtered, and concentrated under reduced pressure to give a residue. The residue was purified by column chromatography (SiO₂, Petroleum ether/Ethyl acetate=10/1 to 5/1). 4 (152 mg, crude) was obtained as a yellow oil.

LCMS: RT=1.727 min, MS cal.: 566.21, 568.21, [M+H]⁺=567.1

### General Procedure for Preparation of Compound 363 (5):

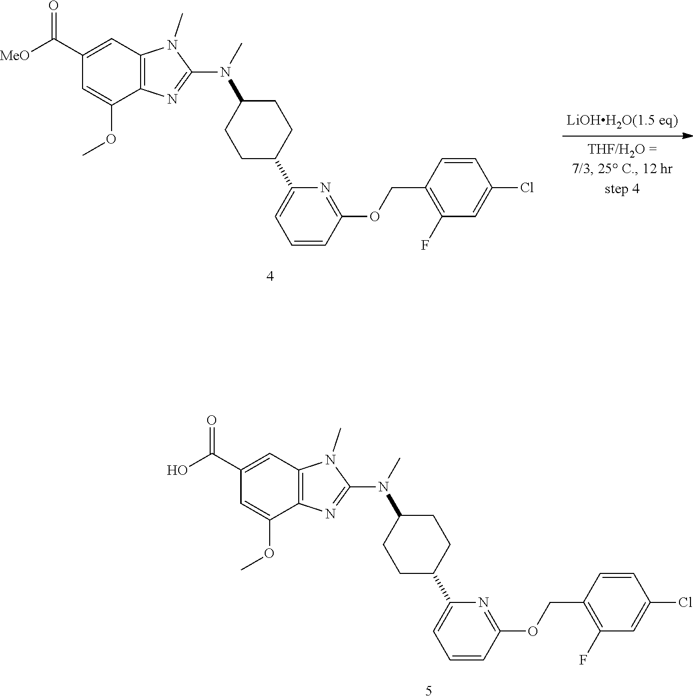 [SMILES: COC(=O)c1cc(OC)c2nc(N(C)[C@H]3CC[C@H](c4cccc(OCc5ccc(Cl)cc5F)n4)CC3)n(C)c2c1 ; COc1cc(C(=O)O)cc2c1nc(N(C)[C@H]1CC[C@H](c3cccc(OCc4ccc(Cl)cc4F)n3)CC1)n2C]

To a solution of 4 (110 mg, 155.19 μmol, 1 eq) in THF (0.7 mL) was added LiOH·H₂O (6.51 mg, 155.19 μmol, 1 eq) in H₂O (0.3 mL). The mixture was stirred at 25° C. for 12 hr. LC-MS showed 4 was consumed completely and desired mass was detected. The reaction mixture was purified directly. The residue was purified by prep-HPLC (neutral condition; column: Waters Xbridge Prep OBD C18 150*40 mm*10 um; mobile phase: [H₂O (10 mM NH₄HCO₃)-ACN]; gradient: 25%-55% B over 8.0 min). 2-(((1r,4r)-4-(6-((4-Chloro-2-fluorobenzyl)oxy)pyridin-2-yl)cyclohexyl)(methyl)amino)-4-methoxy-1-methyl-1H-benzo[d]imidazole-6-carboxylic acid (28.92 mg, 52.29 μmol, 33.70% yield) was obtained as a white solid.

LCMS: RT=2.820 min, MS cal.: 552.19, [M+H]⁺=553.2

HPLC: RT=13.442 min, purity: 95.3%

¹H NMR (400 MHz, MeOH-d4) δ=7.64 (s, 1H), 7.56 (t, J=7.6 Hz, 1H), 7.50 (t, J=8.4 Hz, 1H), 7.44 (s, 1H), 7.27-7.14 (m, 2H), 6.82 (d, J=7.2 Hz, 1H), 6.61 (d, J=8.4 Hz, 1H), 5.40 (s, 2H), 4.00 (s, 3H), 3.70 (s, 3H), 3.49-3.41 (m, 1H), 2.95 (s, 3H), 2.67-2.54 (m, 1H), 2.00 (br d, J=10.4 Hz, 4H), 1.88-1.67 (m, 4H)

### Example A102—Preparation of Compound 364. 2-((4-(2-((4-Chloro-2-fluorobenzyl)oxy)pyridin-3-yl)piperidin-1-yl)methyl)-4-(difluoromethoxy)-1-methyl-1H-benzo[d]imidazole-6-carboxylic acid (Compound 364) (8)

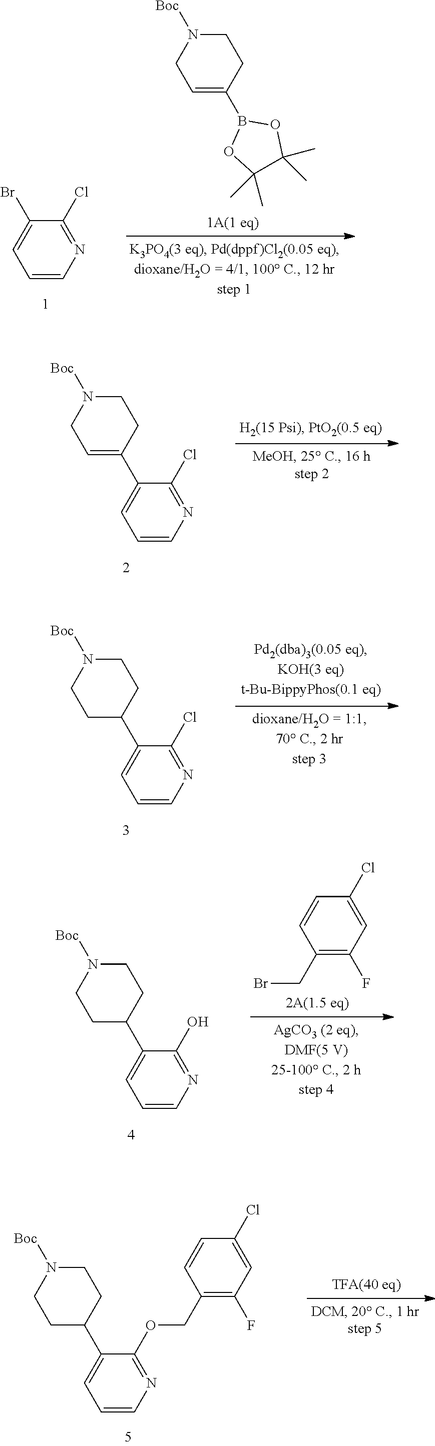

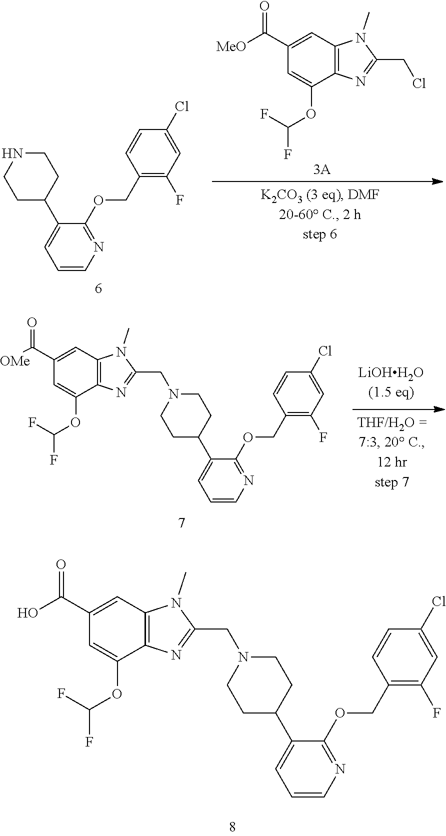 [SMILES: Fc1cc(Cl)ccc1COc1ncccc1C1CCNCC1 ; COC(=O)c1cc(OC(F)F)c2nc(CCl)n(C)c2c1 ; COC(=O)c1cc(OC(F)F)c2nc(CN3CCC(c4cccnc4OCc4ccc(Cl)cc4F)CC3)n(C)c2c1 ; Cn1c(CN2CCC(c3cccnc3OCc3ccc(Cl)cc3F)CC2)nc2c(OC(F)F)cc(C(=O)O)cc21]

### General Procedure for Preparation of Intermediate 2:

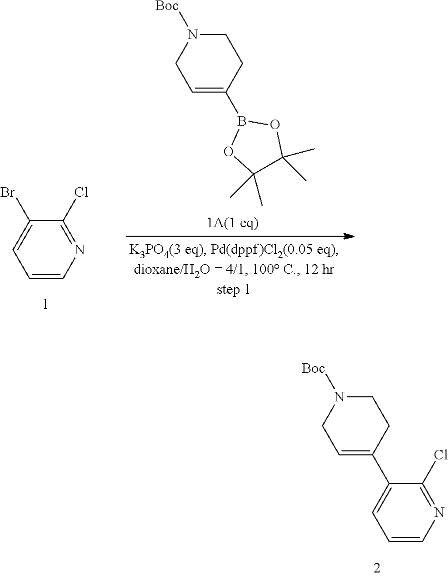 [SMILES: Clc1ncccc1Br ; CC(C)(C)OC(=O)N1CC=C(B2OC(C)(C)C(C)(C)O2)CC1 ; CC(C)(C)OC(=O)N1CC=C(c2cccnc2Cl)CC1]

To a solution of 1 (5 g, 25.98 mmol, 1 eq) and 1A (8.03 g, 25.98 mmol, 1 eq) in dioxane (100 mL) and H₂O (25 mL) was added K₃PO₄ (16.55 g, 77.95 mmol, 3 eq) and Pd(dppf)Cl₂ (950.55 mg, 1.30 mmol, 0.05 eq). The mixture was stirred at 100° C. for 12 hr. The mixture was monitoring by LCMS showed 1 was consumed completely and one main peak with desired m/z was detected. The mixture was filtered by celite pad and filtrate was added water (50 mL), extracted with Ethyl acetate (50 mL*3), the organic phase was washed with brine (50 mL), filtered and concentrated in vacuum to get a residue. The residue was purified by column chromatography (SiO₂, Petroleum ether/Ethyl acetate=1/0 to 10/1). 2 (6.6 g, 22.39 mmol, 86.17% yield, 94% purity) was obtained as a off-white oil.

LCMS: RT=0.529 min, MS cal.: 294.11, 296.11, [M+H]⁺=295.1

¹H NMR (400 MHz, CHCl3-d) δ=8.35-8.28 (m, 1H), 7.55-7.48 (m, 1H), 7.25-7.19 (m, 1H), 5.73 (br s, 1H), 4.07 (br d, J=2.0 Hz, 2H), 3.64 (t, J=5.6 Hz, 2H), 2.46 (br s, 2H), 1.50 (s, 9H)

### General Procedure for Preparation of Intermediate 3:

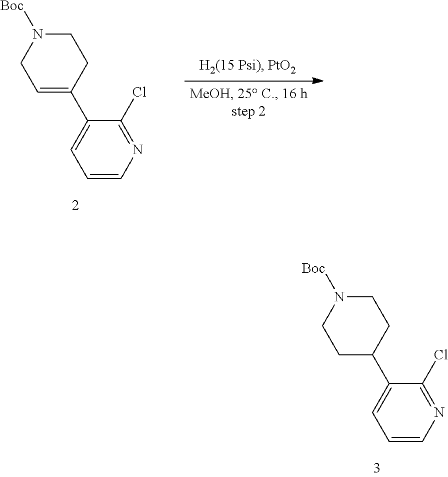 [SMILES: CC(C)(C)OC(=O)N1CC=C(c2cccnc2Cl)CC1 ; CC(C)(C)OC(=O)N1CCC(c2cccnc2Cl)CC1]

MeOH (30 mL) was charged to the 100 mL necked round bottom flask, then PtO₂ (1.16 g) was added at 25° C. At 25° C. inner temperature, 2 (3 g, 10.18 mmol, 1 eq) in MeOH (30 mL) was added to the reaction mixture at 25° C. under H₂ atmosphere. After the addition, the mixture was stirred at 25° C. for 16 hr under H₂ atmosphere. The reaction was monitored by LCMS showed 2 was consumed completely and one main peak with desired m/z was detected. After 16 hr, the reaction mixture was filtered and concentrated under reduced pressure to give a residue. The residue was purified by column chromatography (SiO₂, Petroleum ether/Ethyl acetate=1/0 to 10/1). 3 (1.5 g, 5.05 mmol, 49.66% yield, 99% purity) was obtained as a off-white oil.

LCMS: RT=0.548 min, MS cal.: 296.13, 298.13, [M+H]⁺=297.1

¹H NMR (400 MHz, CHCl3-d) δ=8.29-8.24 (m, 1H), 7.59-7.53 (m, 1H), 7.26-7.21 (m, 1H), 4.29 (br d, J=10.0 Hz, 2H), 3.19-3.05 (m, 1H), 2.86 (br t, J=12.4 Hz, 2H), 1.89 (br d, J=12.8 Hz, 2H), 1.58-1.51 (m, 2H), 1.49 (s, 9H)

### General Procedure for Preparation of Intermediate 4:

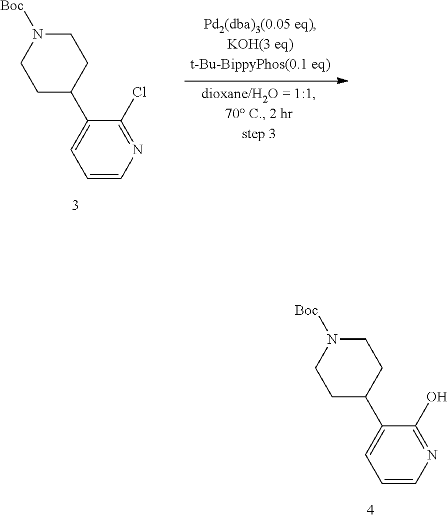 [SMILES: CC(C)(C)OC(=O)N1CCC(c2cccnc2Cl)CC1 ; CC(C)(C)OC(=O)N1CCC(c2cccnc2O)CC1]

A mixture of 3 (1 g, 3.37 mmol, 1 eq), Pd₂(dba)₃ (154.27 mg, 168.47 μmol, 0.05 eq), potassium; hydroxide (567.12 mg, 10.11 mmol, 3 eq) and ditert-butyl-[2-(1,3,5-triphenylpyrazol-4-yl)pyrazol-3-yl] phosphane (170.70 mg, 336.94 μmol, 0.1 eq) in dioxane (10 mL) and H₂O (10 mL) was degassed and purged with N₂ for 3 times, and then the mixture was stirred at 70° C. for 2 hr under N₂ atmosphere. The reaction was monitored by LCMS showed ˜24% of 3 remained and ˜42% of 4 desired Compound was detected. The reaction mixture was diluted with H₂O 50 mL and extracted with EtOAc 60 mL (20 mL*3). The combined organic layers were washed with brine 20 mL, dried over Na₂SO₄, filtered and concentrated under reduced pressure to give a residue. The crude product was triturated with PE:MTBE=1:1 (10 mL) at 25° C. for 5 min, filtered and the filtered cake was concentrated under reduced pressure to give a residue. 4 (670 mg, 2.41 mmol, 71.44% yield, 74% purity) was obtained as a white solid.

LCMS: RT=1.042 min, MS cal.: 278.16, 279.17, [M−H]⁻=277.1

¹H NMR (400 MHz, DMSO-d6) δ=11.51 (br s, 1H), 7.28-7.16 (m, 2H), 6.12 (t, J=6.8 Hz, 1H), 4.04 (br d, J=7.0 Hz, 2H), 2.79 (br t, J=12.0 Hz, 3H), 1.71 (br d, J=11.6 Hz, 2H), 1.40 (s, 9H), 1.38-1.28 (m, 2H)

### General Procedure for Preparation of Intermediate 5:

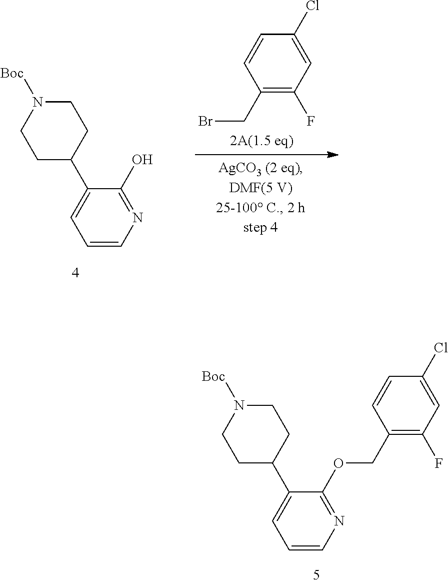 [SMILES: CC(C)(C)OC(=O)N1CCC(c2cccnc2O)CC1 ; Fc1cc(Cl)ccc1CBr ; CC(C)(C)OC(=O)N1CCC(c2cccnc2OCc2ccc(Cl)cc2F)CC1]

A mixture of 4 (500 mg, 1.80 mmol, 1 eq), 2A (602.14 mg, 2.69 mmol, 1.5 eq), Ag₂CO₃ (990.64 mg, 3.59 mmol, 2 eq) in DMF (2.5 mL) was degassed and purged with N₂ for 3 times at 25° C., and then the mixture was stirred at 100° C. for 2 hr under N₂ atmosphere. The mixture was monitoring by LCMS showed 4 was consumed completely and one main peak with desired m/z was detected. The suspension was filtered through diatomite and filter cake was washed with EtOAc 30 ml*3. The combined filtrate were diluted with H₂O 20 mL and extracted with EtOAc 60 mL (20 mL*3). The combined organic layers were washed with brine 20 mL, dried over Na₂SO₄, filtered, and concentrated under reduced pressure to give a residue. The residue was purified by column chromatography (SiO₂, Petroleum ether/Ethyl acetate=100/1 to 10/1). 5 (550 mg, 1.31 mmol, 72.74% yield, 80% purity) was obtained as a white solid.

LCMS: RT=1.745 min, MS cal.: 420.16, 422.16, [M+H]⁺=421.0

¹H NMR (400 MHz, DMSO-d₆) δ=8.04-7.99 (m, 1H), 7.64-7.47 (m, 3H), 7.35-7.28 (m, 1H), 7.03-6.94 (m, 1H), 5.41 (s, 2H), 4.12-3.99 (m, 2H), 2.96-2.85 (m, 1H), 2.87-2.67 (m, 2H), 1.72 (br d, J=12.4 Hz, 2H), 1.54-1.43 (m, 2H), 1.42-1.37 (m, 9H)

### General Procedure for Preparation of Intermediate 6:

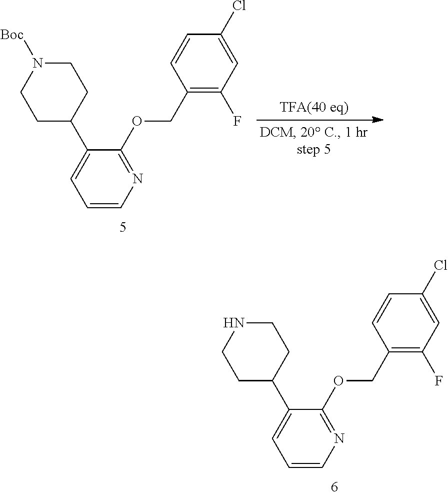 [SMILES: CC(C)(C)OC(=O)N1CCC(c2cccnc2OCc2ccc(Cl)cc2F)CC1 ; Fc1cc(Cl)ccc1COc1ncccc1C1CCNCC1]

To a solution of 5 (550 mg, 1.31 mmol, 1 eq) in DCM (10 mL) was added TFA (5.96 g, 52.27 mmol, 3.88 mL, 40 eq). The mixture was stirred at 20° C. for 1 hr. The reaction was monitored by LCMS showed 5 was consumed completely and one main peak with desired m/z was detected.

The reaction mixture was concentrated under reduced pressure to remove TFA and DCM. The residue was co-evaporated with ACN (10 ml*3). The residue was diluted with ACN 10 mL and added dropwise to sat. NaHCO₃ solution (6 ml) (pH ˜ 8), and then extracted with EtOAc 90 mL (30 mL*3). The combined organic layers were washed with brine 30 mL, dried over Na₂SO₄, filtered, and concentrated under reduced pressure to give a residue. 6 (480 mg, crude, 85% purity) was obtained as a yellow solid.

LCMS: RT=1.124 min, MS cal.: 320.11, 322.11, [M+H]⁺=320.9

¹H NMR (400 MHz, DMSO-d₆) δ=8.04-7.98 (m, 1H), 7.63-7.45 (m, 3H), 7.33-7.29 (m, 1H), 7.00-6.95 (m, 1H), 5.41 (s, 2H), 4.12-3.93 (m, 2H), 2.97-2.85 (m, 1H), 2.78 (br s, 1H), 1.72 (br d, J=12.0 Hz, 2H), 1.56-1.43 (m, 2H), 1.39 (s, 9H)

### General Procedure for Preparation of Intermediate 7:

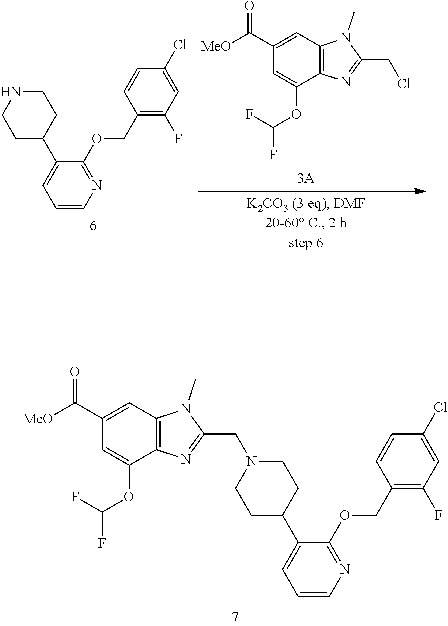 [SMILES: Fc1cc(Cl)ccc1COc1ncccc1C1CCNCC1 ; COC(=O)c1cc(OC(F)F)c2nc(CCl)n(C)c2c1 ; COC(=O)c1cc(OC(F)F)c2nc(CN3CCC(c4cccnc4OCc4ccc(Cl)cc4F)CC3)n(C)c2c1]

To a solution of 6 (150 mg, 467.60 μmol, 1 eq) and 3A (142.47 mg, 467.60 μmol, 1 eq) in DMF (1.5 mL) was added K₂CO₃ (193.88 mg, 1.40 mmol, 3 eq) at 20° C., The mixture was stirred at 60° C. for 2 hr. The reaction was monitored by LCMS showed ˜19% of 6 remained. Several new peaks were shown on LC-MS and ˜32% of desired 7 was detected. The reaction mixture was diluted with H₂O 10 mL and extracted with EtOAc 30 mL (10 mL*3). The combined organic layers were washed with brine 10 mL, dried over Na₂SO₄, filtered, and concentrated under reduced pressure to give a residue. The residue was purified by column chromatography (SiO₂, Petroleum ether/Ethyl acetate=1/0 to 10/1). 7 (80 mg, 135.82 μmol, 29.05% yield, 92% purity) was obtained as a white solid.

LCMS: RT=1.731 min, MS cal.: 588.18, 590.17, [M+H]⁺=589.0

¹H NMR (400 MHz, DMSO-d₆) δ=8.10 (s, 1H), 8.02-7.98 (m, 1H), 7.64 (t, J=74.4 Hz, 1H), 7.59 (d, J=7.2 Hz, 1H), 7.56-7.51 (m, 2H), 7.49 (dd, J=1.6, 10.0 Hz, 1H), 7.32 (dd, J=1.6, 8.4 Hz, 1H), 6.97 (dd, J=5.2, 7.2 Hz, 1H), 5.39 (s, 2H), 3.94 (s, 3H), 3.90 (s, 3H), 3.88-3.81 (m, 2H), 2.93 (d, J=11.2 Hz, 2H), 2.77-2.70 (m, 1H), 2.20 (t, J=10.8 Hz, 2H), 1.77-1.69 (m, 2H), 1.59 (s, 1H), 1.59-1.56 (m, 1H)

### General Procedure for Preparation of Compound 364 (8) Compound 8:

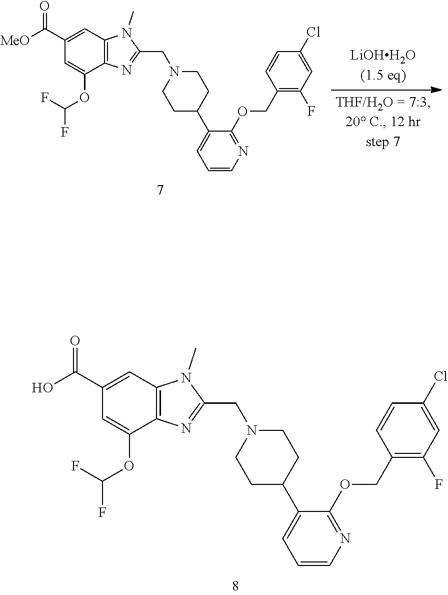 [SMILES: COC(=O)c1cc(OC(F)F)c2nc(CN3CCC(c4cccnc4OCc4ccc(Cl)cc4F)CC3)n(C)c2c1 ; Cn1c(CN2CCC(c3cccnc3OCc3ccc(Cl)cc3F)CC2)nc2c(OC(F)F)cc(C(=O)O)cc21]

To a solution of 7 (60 mg, 101.87 μmol, 1 eq) in THF (0.7 mL) was added LiOH·H₂O (6.41 mg, 152.80 μmol, 1.5 eq) in H₂O (0.3 mL). The mixture was stirred at 20° C. for 12 hr. The mixture was monitoring by LCMS showed 7 was consumed completely and one main peak with desired m/z was detected. The mixture was purified by prep-HPLC (column: Waters Xbridge BEH C18 100*30 mm*10 um; mobile phase: [H₂O (10 mM NH₄HCO₃)-ACN]; gradient: 20%-65% B over 8.0 min). 2-((4-(2-((4-Chloro-2-fluorobenzyl)oxy)pyridin-3-yl)piperidin-1-yl)methyl)-4-(difluoromethoxy)-1-methyl-1H-benzo[d]imidazole-6-carboxylic acid (28.97 mg, 50.38 μmol, 49.46% yield, 97% purity) was obtained as a white solid.

LCMS: RT=2.445 min, MS cal.: 574.16, 576.16, [M+H]⁺=575.1

HPLC: RT=11.993 min, purity: 97.40%

¹H NMR (400 MHz, DMSO-d₆) δ=8.06 (d, J=1.2 Hz, 1H), 8.02-7.99 (m, 1H), 7.62 (t, J=74.4 Hz, 1H), 7.61-7.57 (m, 1H), 7.57-7.47 (m, 3H), 7.35-7.30 (m, 1H), 6.99-6.94 (m, 1H), 5.39 (s, 2H), 3.92 (s, 3H), 3.84 (s, 2H), 2.93 (d, J=11.2 Hz, 2H), 2.80-2.69 (m, 1H), 2.20 (t, J=10.8 Hz, 2H), 1.76-1.70 (m, 2H), 1.70-1.57 (m, 2H)

### Example A103—Preparation of Compound 308. 2-((4-(6-((4-Cyano-2-fluorobenzyl)oxy)pyridin-2-yl)piperidin-1-yl)methyl)-4-methoxy-1-methyl-1H-benzo[d]imidazole-6-carboxylic acid (Compound 308) (3)

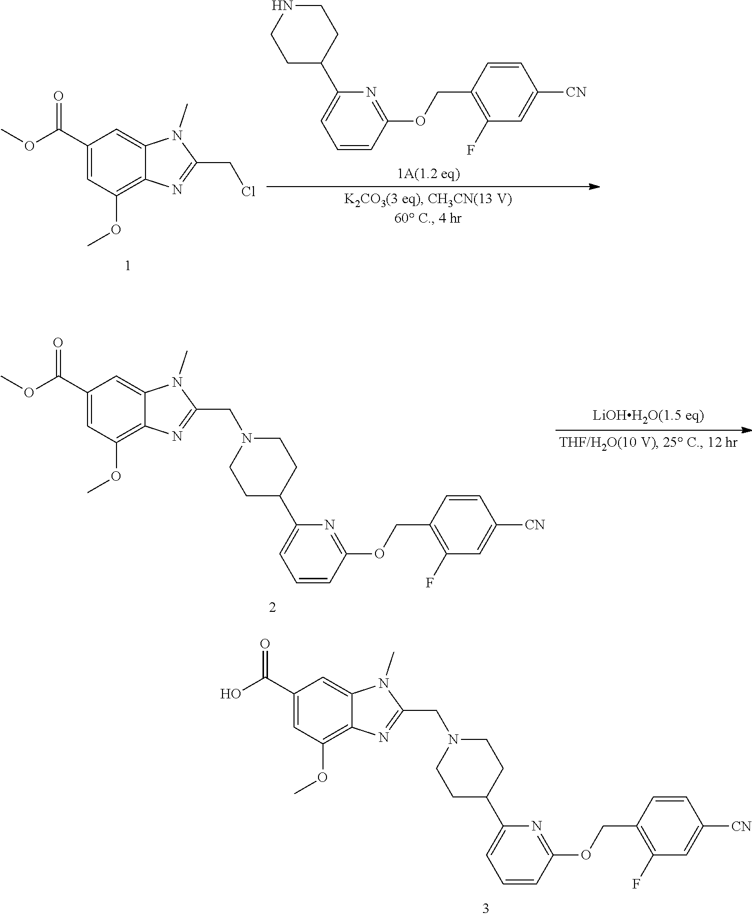 [SMILES: COC(=O)c1cc(OC)c2nc(CCl)n(C)c2c1 ; N#Cc1ccc(COc2cccc(C3CCNCC3)n2)c(F)c1 ; COC(=O)c1cc(OC)c2nc(CN3CCC(c4cccc(OCc5ccc(C#N)cc5F)n4)CC3)n(C)c2c1 ; COc1cc(C(=O)O)cc2c1nc(CN1CCC(c3cccc(OCc4ccc(C#N)cc4F)n3)CC1)n2C]

### General Procedure for Preparation of Intermediate 2:

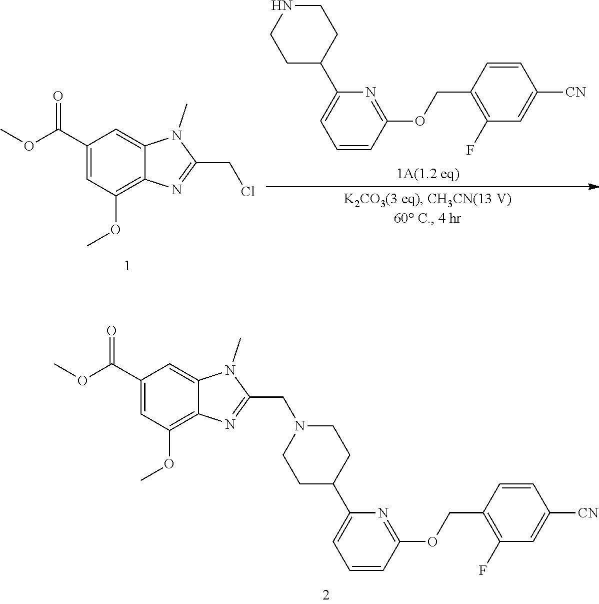 [SMILES: COC(=O)c1cc(OC)c2nc(CCl)n(C)c2c1 ; N#Cc1ccc(COc2cccc(C3CCNCC3)n2)c(F)c1 ; COC(=O)c1cc(OC)c2nc(CN3CCC(c4cccc(OCc5ccc(C#N)cc5F)n4)CC3)n(C)c2c1]

A mixture of 1 (150 mg, 558.25 μmol, 1 eq), 1A (208.58 mg, 669.90 μmol, 1.2 eq), K₂CO₃ (231.46 mg, 1.67 mmol, 3 eq), in CH₃CN (2 mL) was degassed and purged with N₂ for 3 times, and then the mixture was stirred at 60° C. for 4 hr under N₂ atmosphere. LC-MS showed 1 was consumed completely and one main peak with desired mass was detected. The reaction mixture was diluted with H₂O 10 mL and extracted with EtOAc 45 mL (15 mL*3). The combined organic layers were washed with brine 10 mL, dried over Na₂SO₄, filtered, and concentrated under reduced pressure to give a residue. The residue was purified by column chromatography (SiO₂, Petroleum ether/Ethyl acetate=10/1 to 0/1). 2 (180 mg, 331.13 μmol, 59.32% yield) was obtained as a white solid.

LCMS: RT=0.423 min, MS cal.: 543.60, [M+1]⁺=544.2

¹H NMR (400 MHz, CDCl₃) δ=7.81-7.77 (m, 1H), 7.63 (br t, J=7.3 Hz, 1H), 7.53 (t, J=7.7 Hz, 1H), 7.44 (d, J=8.0 Hz, 1H), 7.41-7.35 (m, 2H), 6.75 (d, J=7.3 Hz, 1H), 6.64 (d, J=8.1 Hz, 1H), 5.50 (s, 2H), 4.07 (s, 3H), 3.97 (s, 6H), 3.88 (br s, 2H), 3.00-2.93 (m, 2H), 2.67-2.54 (m, 1H), 2.28 (br s, 2H), 1.84 (br s, 3H), 1.65-1.53 (m, 1H)

### General Procedure for Preparation of Compound 308 (3):

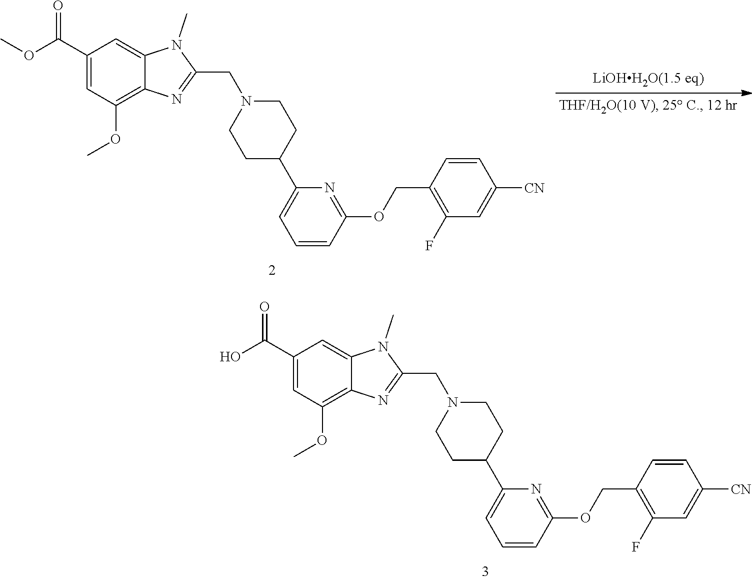 [SMILES: COC(=O)c1cc(OC)c2nc(CN3CCC(c4cccc(OCc5ccc(C#N)cc5F)n4)CC3)n(C)c2c1 ; COc1cc(C(=O)O)cc2c1nc(CN1CCC(c3cccc(OCc4ccc(C#N)cc4F)n3)CC1)n2C]

A mixture of 2 (160 mg, 294.34 μmol, 1 eq), LiOH·H₂O (18.53 mg, 441.51 μmol, 1.5 eq), in THF (1.4 mL) and H₂O (0.6 mL) was degassed and purged with N₂ for 3 times, and then the mixture was stirred at 25° C. for 12 hr under N₂ atmosphere. LC-MS (ET83888-106, product, RT=1.069 min) showed 2 was consumed completely and one main peak with desired mass was detected. The reaction was purified directly. The residue was purified by prep-HPLC (column: Waters Xbridge Prep OBD C18 150*40 mm*10 um; mobile phase: [H₂O (10 mM NH₄HCO₃)-ACN]; gradient: 20%-60% B over 8.0 min). 2-((4-(6-((4-Cyano-2-fluorobenzyl)oxy)pyridin-2-yl)piperidin-1-yl)methyl)-4-methoxy-1-methyl-1H-benzo[d]imidazole-6-carboxylic acid (29.91 mg, 56.48 μmol, 19.19% yield) was obtained as a white solid.

LCMS: RT=2.188 min, MS cal.: 529.57, [M+1]⁺=530.2

HPLC: RT=9.84 min, purity: 99.80%

¹H NMR (400 MHz, MEOD) δ=7.84 (s, 1H), 7.67 (t, J=7.2 Hz, 1H), 7.61-7.50 (m, 3H), 7.45 (s, 1H), 6.83 (d, J=7.2 Hz, 1H), 6.67 (d, J=8.4 Hz, 1H), 5.50 (s, 2H), 4.03 (s, 3H), 3.98 (s, 3H), 3.89 (s, 2H), 3.01 (br d, J=11.2 Hz, 2H), 2.69-2.56 (m, 1H), 2.36-2.24 (m, 2H), 1.88-1.77 (m, 4H)

### Example A104—Preparation of Compound 365. 2-((4-(6-((4-Cyclopropyl-2-fluorobenzyl)oxy)pyridin-2-yl)piperidin-1-yl)methyl)-4-(difluoromethoxy)-1-methyl-1H-benzo[d]imidazole-6-carboxylic acid (Compound 365) (3)

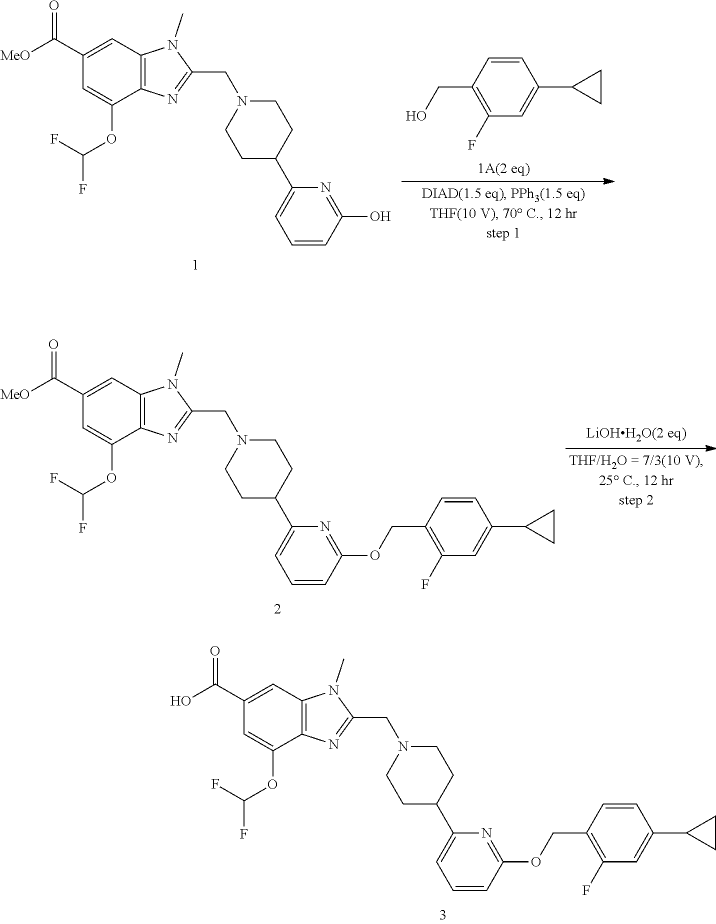 [SMILES: COC(=O)c1cc(OC(F)F)c2nc(CN3CCC(c4cccc(O)n4)CC3)n(C)c2c1 ; OCc1ccc(C2CC2)cc1F ; COC(=O)c1cc(OC(F)F)c2nc(CN3CCC(c4cccc(OCc5ccc(C6CC6)cc5F)n4)CC3)n(C)c2c1 ; Cn1c(CN2CCC(c3cccc(OCc4ccc(C5CC5)cc4F)n3)CC2)nc2c(OC(F)F)cc(C(=O)O)cc21]

### General Procedure for Preparation of Intermediate 2:

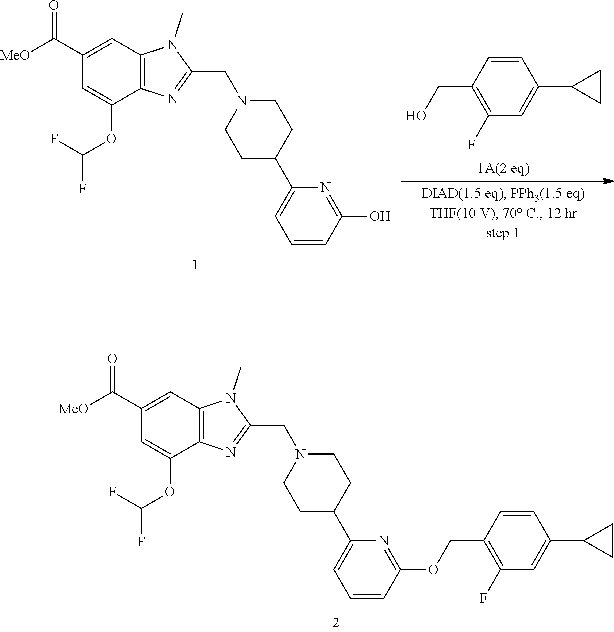 [SMILES: COC(=O)c1cc(OC(F)F)c2nc(CN3CCC(c4cccc(O)n4)CC3)n(C)c2c1 ; OCc1ccc(C2CC2)cc1F ; COC(=O)c1cc(OC(F)F)c2nc(CN3CCC(c4cccc(OCc5ccc(C6CC6)cc5F)n4)CC3)n(C)c2c1]

To a solution of 1 (150 mg, 335.99 μmol, 1 eq) and 1A (111.68 mg, 671.97 μmol, 2 eq) in THF (1.5 mL) was added DIAD (101.91 mg, 503.98 μmol, 97.71 μL, 1.5 eq) and PPh₃ (132.19 mg, 503.98 μmol, 1.5 eq). The mixture was stirred at 70° C. for 12 hr. The mixture was monitoring by LCMS showed 1 was consumed completely and one main peak with desired m/z was detected. TLC (Petroleum ether:Ethyl acetate=0:1) indicated 1 was consumed completely and one new spot formed. The reaction was clean according to TLC. H₂O 10 mL was added to the reaction mixture at 0° C. The mixture was filtered first, then extracted by Ethyl acetate (10 mL*3). Then organic phase was combined and washed by H₂O (5 mL), brine (5 mL), dried with Na₂SO₄, filtered. The organic phase was concentrated under reduced pressure at 45° C. to give a residue. The residue was purified by column chromatography (SiO₂, Petroleum ether/Ethyl acetate=100/0 to 1/1). And the crude product was also purified by prep-HPLC (column: Waters Xbridge BEH C18 100*30 mm*10 um; mobile phase: [H₂O (10 mM NH₄HCO₃)-ACN]; gradient: 60%-100% B over 8.0 min). 2 (40 mg, 63.91 μmol, 19.02% yield, 95% purity) was obtained as a white solid.

LCMS: RT=1.778 min, MS cal.: 594.25, 595.25, [M+H]⁺=595.2

### General Procedure for Preparation of Compound 365 (3):

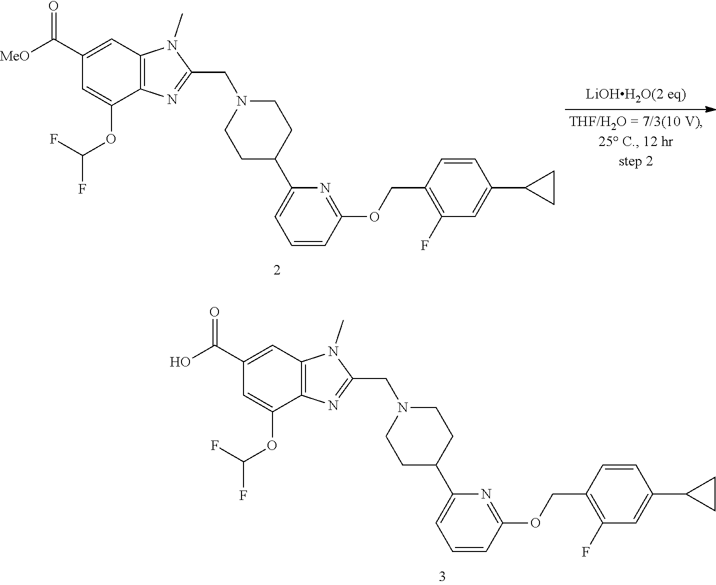 [SMILES: COC(=O)c1cc(OC(F)F)c2nc(CN3CCC(c4cccc(OCc5ccc(C6CC6)cc5F)n4)CC3)n(C)c2c1 ; Cn1c(CN2CCC(c3cccc(OCc4ccc(C5CC5)cc4F)n3)CC2)nc2c(OC(F)F)cc(C(=O)O)cc21]

To a solution of 2 (40 mg, 67.27 μmol, 1 eq) in THF (0.28 mL) and H₂O (0.12 mL) was added LiOH·H₂O (5.65 mg, 134.54 μmol, 2 eq). The mixture was stirred at 25° C. for 12 hr. The reaction was monitored by LCMS showed 2 was consumed completely and one main peak with desired m/z was detected. The residue was purified by prep-HPLC (column: Waters Xbridge BEH C18 100*30 mm*10 um; mobile phase: [H₂O (10 mM NH₄HCO₃)-ACN]; gradient: 25%-65% B over 8.0 min). 2-((4-(6-((4-Cyclopropyl-2-fluorobenzyl)oxy)pyridin-2-yl)piperidin-1-yl)methyl)-4-(difluoromethoxy)-1-methyl-1H-benzo[d]imidazole-6-carboxylic acid (19.19 mg, 33.05 μmol, 49.13% yield, 100% purity) was obtained as a white solid.

LCMS: RT=2.863 min, MS cal.: 580.23, 581.23, [M+H]⁺=581.3

HPLC: RT=12.793 min, purity: 100.00%

¹H NMR (400 MHz, DMSO-d₄) δ=8.08 (d, J=1.2 Hz, 1H), 7.64 (t, J=74.8 Hz, 1H), 7.63-7.59 (m, 1H), 7.52 (s, 1H), 7.39 (t, J=8.0 Hz, 1H), 6.91 (s, 1H), 6.89 (dd, J=1.2, 5.6 Hz, 1H), 6.87-6.84 (m, 1H), 6.63 (d, J=8.0 Hz, 1H), 5.31 (s, 2H), 3.98 (s, 3H), 3.86 (s, 2H), 2.95 (br d, J=11.2 Hz, 2H), 2.65-2.54 (m, 1H), 2.28-2.18 (m, 2H), 1.95-1.87 (m, 1H), 1.83-1.77 (m, 2H), 1.77-1.68 (m, 2H), 0.99-0.90 (m, 2H), 0.69-0.64 (m, 2H)

### Example A105—Preparation of Compound 366. 2-((4-(6-((4-Cyano-2,5-difluorobenzyl)oxy)pyridin-2-yl)piperidin-1-yl)methyl)-4-(difluoromethoxy)-1-methyl-1H-benzo[d]imidazole-6-carboxylic acid (Compound 366) (4)

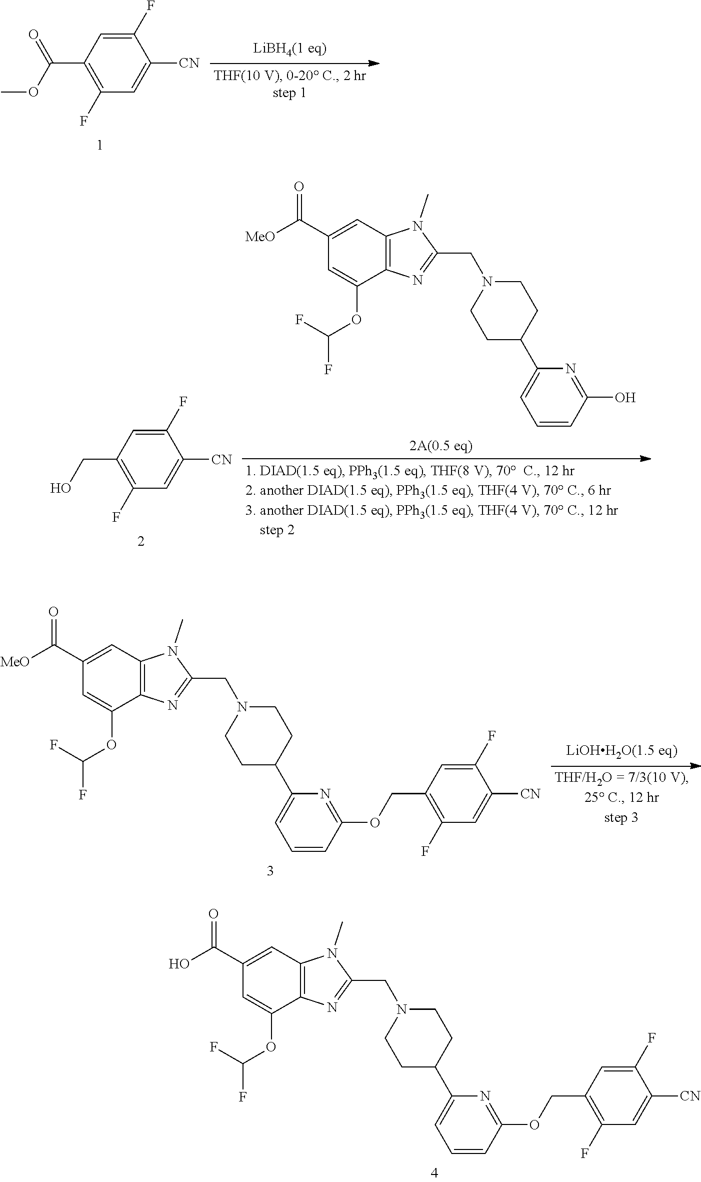 [SMILES: COC(=O)c1cc(F)c(C#N)cc1F ; N#Cc1cc(F)c(CO)cc1F ; COC(=O)c1cc(OC(F)F)c2nc(CN3CCC(c4cccc(O)n4)CC3)n(C)c2c1 ; COC(=O)c1cc(OC(F)F)c2nc(CN3CCC(c4cccc(OCc5cc(F)c(C#N)cc5F)n4)CC3)n(C)c2c1 ; Cn1c(CN2CCC(c3cccc(OCc4cc(F)c(C#N)cc4F)n3)CC2)nc2c(OC(F)F)cc(C(=O)O)cc21]

### General Procedure for Preparation of Intermediate 2:

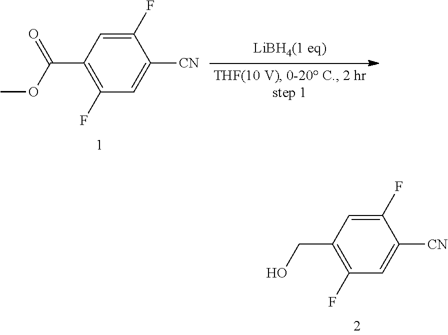

Equip a 25 mL three-necked round bottom flask, thermometer and N₂ balloon. THF (3 mL) was charged to the 25 mL three-necked round bottom flask, then 1 (300.00 mg, 1.52 mmol, 1 eq) was added at 25° C. At 0° C. (inner temperature), LiBH₄ (2 M, 760.89 μL, 1 eq) was added to the reaction mixture at 0° C. After the addition, the mixture was stirred at 20° C. for 2 hr. The reaction was monitored by TLC. TLC (Petroleum ether/Ethyl acetate=3/1, product R_{f}=0.30) indicated 1 was consumed completely and one new spot formed. The reaction was clean according to TLC. The reaction mixture was added to a.q. NH₄Cl 10 mL at 20° C. The mixture was extracted by EtOAc 30 mL (10 mL*3). Then organic phase was washed brine 10 mL. The organic was dried over Na₂SO₄, filtered. The organic phase was concentrated under reduced pressure at 45° C. to give a residue. The residue was purified by column chromatography (SiO₂, Petroleum ether/Ethyl acetate=10/1 to 2/1). 2 (160 mg, 946.03 μmol, 62% yield) was obtained as a yellow solid.

LCMS: RT=0.829 min, MS cal.: 169.1, [M−H]⁺=168.0

¹H NMR (400 MHz, DMSO-d6) δ=7.91 (dd, J=5.2, 9.2 Hz, 1H), 7.53 (dd, J=5.6, 9.6 Hz, 1H), 5.68 (t, J=5.6 Hz, 1H), 4.61 (d, J=5.6 Hz, 2H)

### General Procedure for Preparation of Intermediate 3:

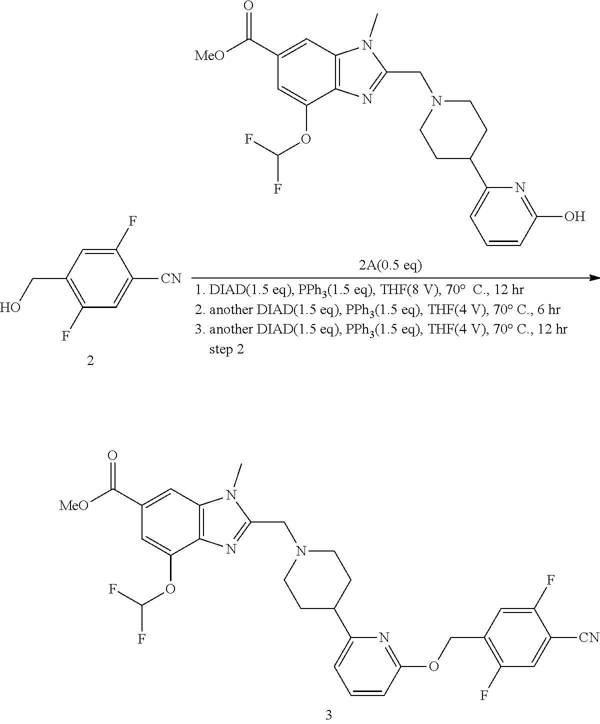 [SMILES: N#Cc1cc(F)c(CO)cc1F ; COC(=O)c1cc(OC(F)F)c2nc(CN3CCC(c4cccc(O)n4)CC3)n(C)c2c1 ; COC(=O)c1cc(OC(F)F)c2nc(CN3CCC(c4cccc(OCc5cc(F)c(C#N)cc5F)n4)CC3)n(C)c2c1]

To a solution of 2A (131.98 mg, 295.63 μmol, 1 eq) and 2 (100 mg, 591.27 μmol, 2 eq) in THF (0.4 mL) was added a solution of DIAD (89.67 mg, 443.45 μmol, 85.97 μL, 1.5 eq) and PPh₃ (116.31 mg, 443.45 μmol, 1.5 eq) in THF (0.4 mL) at 20° C. The mixture was stirred at 70° C. for 12 hr. LC-MS showed ˜17% of 2A remained. Several new peaks were shown on LC-MS and ˜21% of desired Compound was detected. The mixture was added a solution of DIAD (89.67 mg, 443.45 μmol, 85.97 μL, 1.5 eq) and PPh₃ (116.31 mg, 443.45 μmol, 1.5 eq) in THF (0.4 mL) and stirred at 70° C. for 6 hr. LC-MS showed ˜5% of 2A remained. Several new peaks were shown on LC-MS and ˜26% of desired Compound was detected. The mixture was added a solution of DIAD (89.67 mg, 443.45 μmol, 85.97 μL, 1.5 eq) and PPh₃ (116.31 mg, 443.45 μmol, 1.5 eq) in THF (0.4 mL) and stirred at 70° C. for 12 hr. LC-MS (RT=1.626) showed 2 was consumed completely and desired mass was detected. The reaction mixture was diluted with H₂O 10 mL and extracted with EtOAc 30 mL (10 mL*3). The combined organic layers were washed with brine 10 mL, dried over Na₂SO₄, filtered and concentrated under reduced pressure to give a residue. The residue was purified by prep-HPLC (neutral condition, column: Waters Xbridge Prep OBD C18 150*40 mm*10 um; mobile phase: [H₂O (10 mM NH₄HCO₃)-ACN]; gradient: 65%-95% B over 8.0 min). 3 (60 mg, 100.41 μmol, 33.96% yield) was obtained as a white solid.

LCMS: RT=1.643 min, MS cal.: 597.2, 598.2, [M+H]⁺=598.1, 599.1

¹H NMR (400 MHz, CHCl3-d) δ=7.99 (d, J=1.2 Hz, 1H), 7.72 (s, 1H), 7.55 (t, J=7.6 Hz, 1H), 7.40 (dd, J=5.6, 8.8 Hz, 1H), 7.35-7.30 (m, 1H), 7.31 (t, J=74.4 Hz, 1H), 6.78 (d, J=7.2 Hz, 1H), 6.67 (d, J=8.4 Hz, 1H), 5.49 (s, 2H), 4.00 (s, 3H), 3.97 (s, 3H), 3.89 (s, 2H), 2.96 (d, J=9.6 Hz, 2H), 2.66-2.54 (m, 1H), 2.30 (t, J=10.8 Hz, 2H), 1.91-1.82 (m, 2H), 1.82-1.68 (m, 2H)

### General Procedure for Preparation of Compound 366 (4):

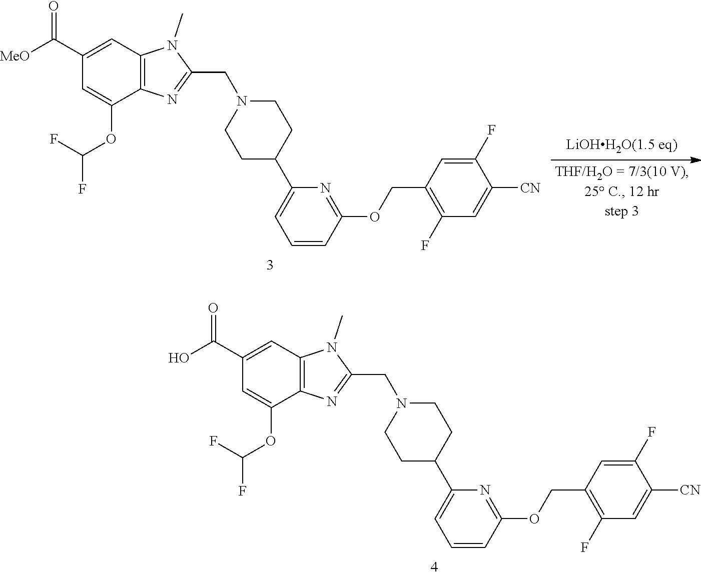 [SMILES: COC(=O)c1cc(OC(F)F)c2nc(CN3CCC(c4cccc(OCc5cc(F)c(C#N)cc5F)n4)CC3)n(C)c2c1 ; Cn1c(CN2CCC(c3cccc(OCc4cc(F)c(C#N)cc4F)n3)CC2)nc2c(OC(F)F)cc(C(=O)O)cc21]

To a solution of 3 (60 mg, 100.41 μmol, 1 eq) in THF (0.7 mL) was added LiOH·H₂O (6.32 mg, 150.61 μmol, 1.5 eq) in H₂O (0.3 mL). The mixture was stirred at 25° C. for 12 hr. LCMS (product RT=1.184) showed 3 was consumed completely and desired mass was detected. The mixture was purified directly. The residue was purified by prep-HPLC (neutral condition, column: Waters Xbridge BEH C18 100*30 mm*10 um; mobile phase: [H₂O (10 mM NH₄HCO₃)-ACN]; gradient: 21%-56% B over 8.0 min). 2-((4-(6-((4-Cyano-2,5-difluorobenzyl)oxy)pyridin-2-yl)piperidin-1-yl)methyl)-4-(difluoromethoxy)-1-methyl-1H-benzo[d]imidazole-6-carboxylic acid (21.22 mg, 36.31 μmol, 36.16% yield, 99.85% purity) was obtained as a white solid.

LCMS: RT=2.688 min, MS cal.: 583.2, 584.2, [M+H]⁺=584.2, 585.2

HPLC: RT=11.289 min, purity: 99.85%

¹H NMR (400 MHz, DMSO-d6) δ=8.09-8.06 (m, 1H), 8.04-7.98 (m, 1H), 7.69-7.64 (m, 2H), 7.63 (t, J=74.4 Hz, 1H), 7.52 (s, 1H), 6.89 (d, J=7.2 Hz, 1H), 6.73 (d, J=8.4 Hz, 1H), 5.43 (s, 2H), 3.97 (s, 3H), 3.85 (s, 2H), 2.92 (d, J=11.2 Hz, 2H), 2.61-2.54 (m, 1H), 2.21 (t, J=10.8 Hz, 2H), 1.80-1.72 (m, 2H), 1.71-1.59 (m, 2H)

### Example A106—Preparation of Compound 367. 2-(4-((6-((4-chloro-2-fluorophenoxy)methyl)pyridin-2-yl)oxy)-2-fluorobenzyl)-4-(difluoromethoxy)-1-methyl-1H-benzo[d]imidazole-6-carboxylic acid (Compound 367) (6)

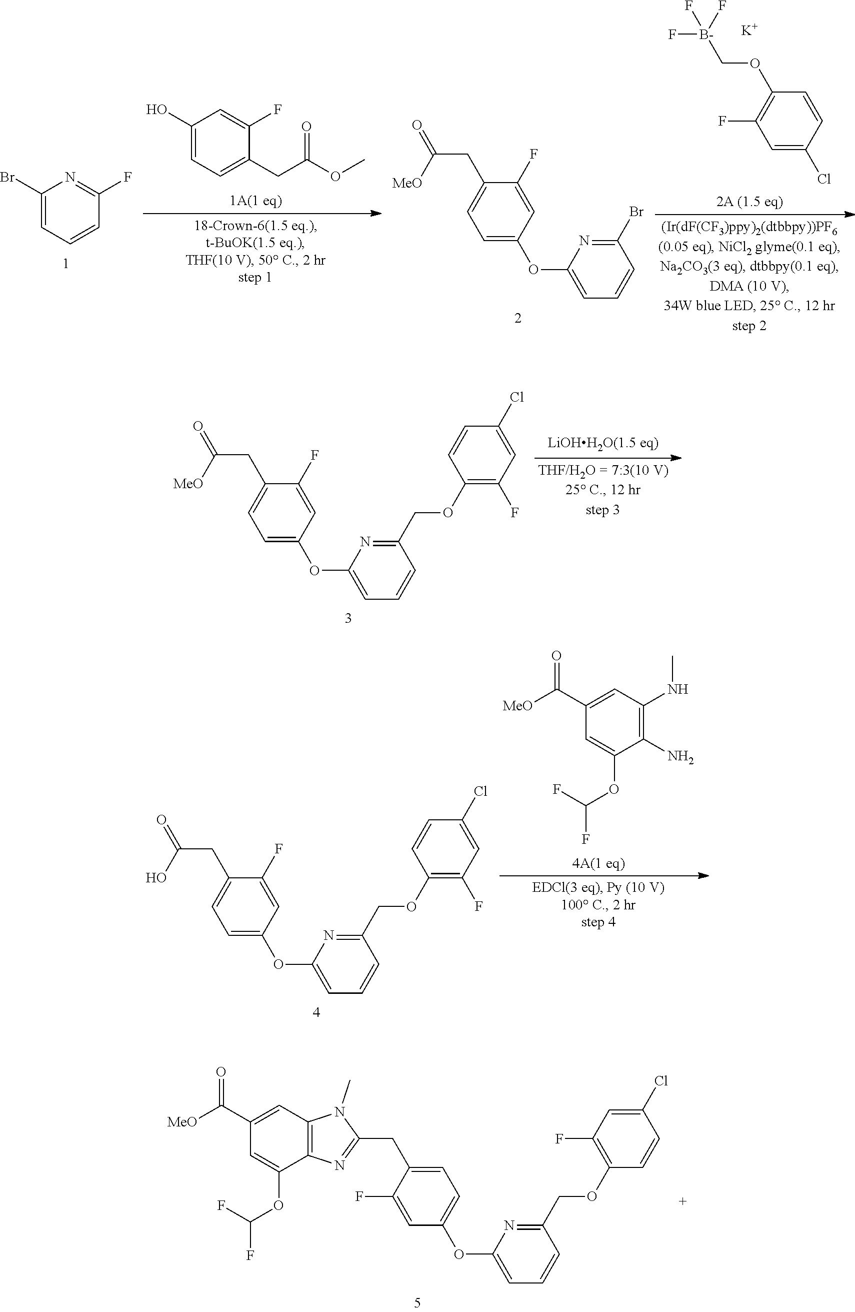

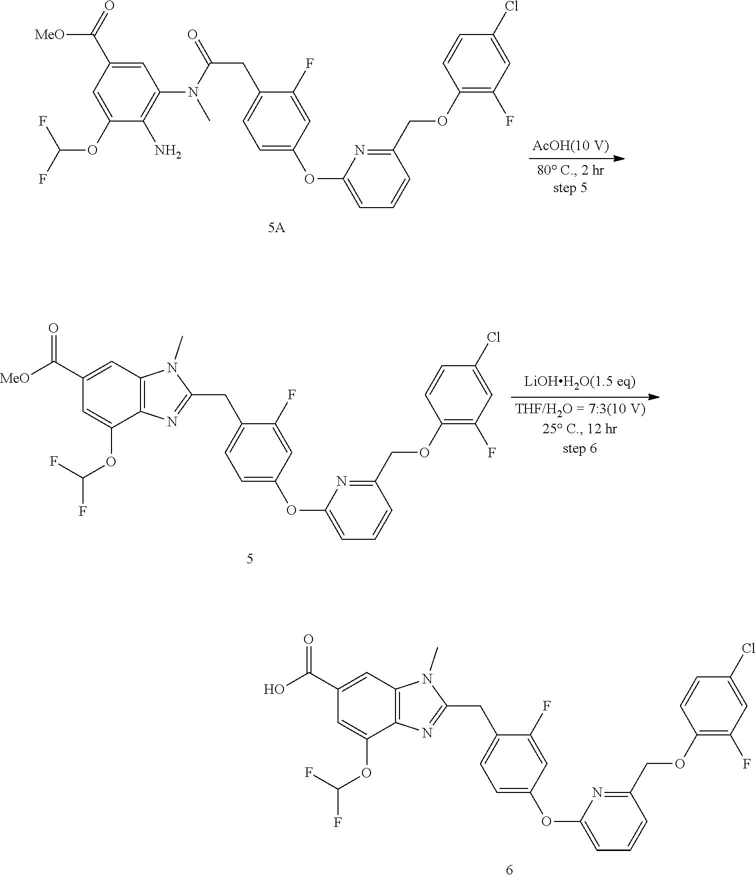 [SMILES: COC(=O)c1cc(OC(F)F)c(N)c(N(C)C(=O)Cc2ccc(Oc3cccc(COc4ccc(Cl)cc4F)n3)cc2F)c1 ; COC(=O)c1cc(OC(F)F)c2nc(Cc3ccc(Oc4cccc(COc5ccc(Cl)cc5F)n4)cc3F)n(C)c2c1 ; Cn1c(Cc2ccc(Oc3cccc(COc4ccc(Cl)cc4F)n3)cc2F)nc2c(OC(F)F)cc(C(=O)O)cc21]

### General Procedure for Preparation of Intermediate 2:

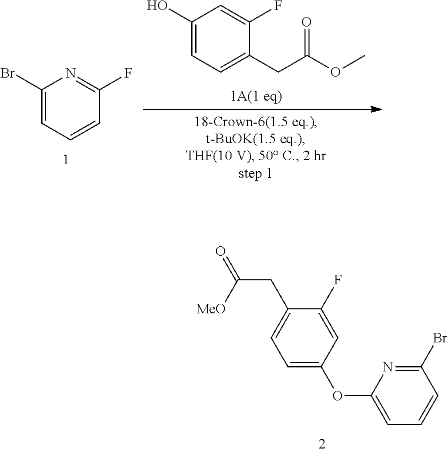 [SMILES: COC(=O)Cc1ccc(O)cc1F ; Fc1cccc(Br)n1 ; COC(=O)Cc1ccc(Oc2cccc(Br)n2)cc1F]

To a solution of 1 (1 g, 5.68 mmol, 1 eq) in THF (10 mL) was added 18-crown-6 (2.25 g, 8.52 mmol, 1.5 eq), potassium; 2-methylpropan-2-olate (956.43 mg, 8.52 mmol, 1.5 eq) and 1A (1.05 g, 5.68 mmol, 1 eq). The mixture was stirred at 50° C. for 2 hr. TLC indicated 1 was consumed completely and one new spot formed. The reaction was clean according to TLC. The reaction mixture was added 1M FA 2 mL. Then the mixture was diluted with H₂O 10 mL and extracted with EtOAc 20 mL (10 mL*2). The combined organic layers were washed with brine 10 mL, dried over Na₂SO₄, filtered, and concentrated under reduced pressure to give a residue. The residue was purified by column chromatography (SiO₂, PE/EtOAc=1/0 to 5/1). 2 (710 mg, 2.05 mmol, 36.00% yield) was obtained as a colorless oil.

LCMS: RT=1.354 min, MS cal.: 339.0/341.0, [M+1]⁺=339.9/341.9

¹HNMR (400 MHz, CHCl3-d) δ=7.55 (t, J=8.0 Hz, 1H), 7.32-7.23 (m, 2H), 6.95-6.90 (m, 2H), 6.85 (d, J=8.0 Hz, 1H), 3.74 (s, 3H), 3.68 (s, 2H)

### General Procedure for Preparation of Intermediate 3:

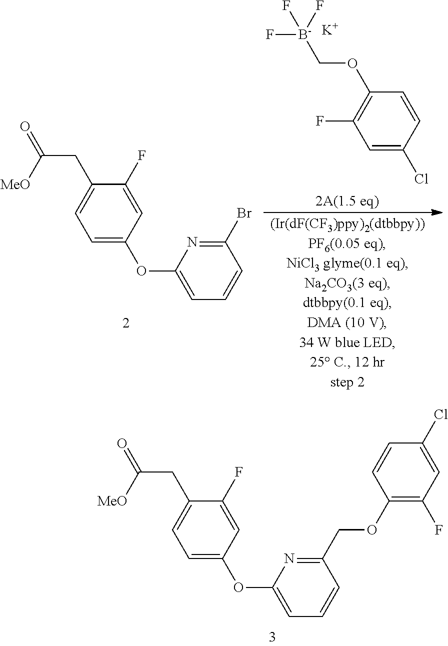 [SMILES: COC(=O)Cc1ccc(Oc2cccc(Br)n2)cc1F ; Fc1cc(Cl)ccc1OC[B-](F)(F)F ; [K+] ; COC(=O)Cc1ccc(Oc2cccc(COc3ccc(Cl)cc3F)n2)cc1F]

To a solution of 2 (500 mg, 1.47 mmol, 1 eq) in DMA (5 mL) was added (Ir(dF(CF₃)ppy)2(dtbbpy))PF6 (82.46 mg, 73.50 μmol, 0.05 eq), Na₂CO₃ (467.40 mg, 4.41 mmol, 3 eq), NiCl₂ glyme (39.45 mg, 147.00 μmol, 0.1 eq), dtbbpy (32.30 mg, 147.00 μmol, 0.1 eq) and Compound 2A (587.55 mg, 2.20 mmol, 1.5 eq, K⁺). The mixture was stirred at 25° C. for 12 hr. LCMS showed 2 was consumed completely and one main peak with desired mass was detected. The reaction mixture was diluted with H₂O 10 mL and extracted with EtOAc 30 mL (10 mL*3). The combined organic layers were washed with brine 10 mL, dried over Na₂SO₄, filtered, and concentrated under reduced pressure to give a residue. The residue was purified by column chromatography (SiO₂, PE/EtOAc=1/0 to 9/1). 3 (370 mg, 749.16 μmol, 50.96% yield) was obtained as a colorless oil.

LCMS: RT=1.556 min, MS cal.: 419.1/421.1, [M+1]⁺=419.9/421.9

¹HNMR (400 MHz, CHCl3-d) δ=7.78-7.72 (m, 1H), 7.32-7.27 (m, 2H), 7.26 (br s, 1H), 7.15-7.10 (m, 1H), 7.02-6.98 (m, 1H), 6.94-6.88 (m, 3H), 6.84 (d, J=8.0 Hz, 1H), 5.11 (s, 2H), 3.75 (s, 3H), 3.69 (s, 2H)

### General Procedure for Preparation of Intermediate 4:

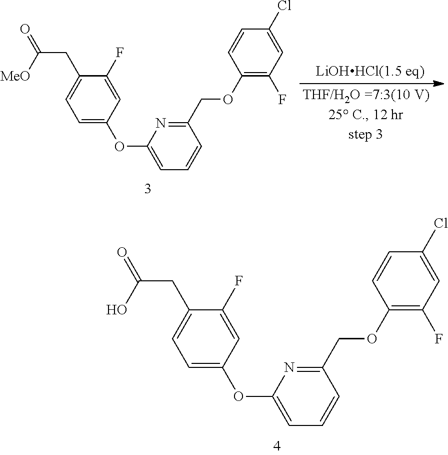 [SMILES: COC(=O)Cc1ccc(Oc2cccc(COc3ccc(Cl)cc3F)n2)cc1F ; O=C(O)Cc1ccc(Oc2cccc(COc3ccc(Cl)cc3F)n2)cc1F]

To a solution of 3 (370 mg, 881.36 μmol, 1 eq) in THF (2.8 mL) and H₂O (1.2 mL) was added LiOH·H₂O (55.48 mg, 1.32 mmol, 1.5 eq). The mixture was stirred at 25° C. for 12 hr. LCMS showed 3 was consumed completely and one main peak with desired mass was detected. The reaction mixture was adjusted to pH=4 with 0.5M citric acid, then the mixture was diluted with H₂O 5 mL and extracted with EtOAc 20 mL (10 mL*2). The combined organic layers were washed with brine 10 mL, dried over Na₂SO₄, filtered and concentrated under reduced pressure to give a residue. 4 (290 mg, 678.94 μmol, 77.03% yield) was obtained as a colorless oil.

LCMS: RT=1.069 min, MS cal.: 405.1/407.1, [M+1]⁺=406.1/407.9

¹HNMR (400 MHz, DMSO-d6) δ=7.93 (t, J=8.0 Hz, 1H), 7.46-7.42 (m, 1H), 7.38-7.32 (m, 1H), 7.28 (d, J=7.2 Hz, 1H), 7.25-7.19 (m, 1H), 7.16-7.12 (m, 1H), 7.07-7.00 (m, 2H), 6.93 (dd, J=2.4, 8.4 Hz, 1H), 5.12 (s, 2H), 3.62 (s, 2H)

### General Procedure for Preparation of Intermediate 5&5A:

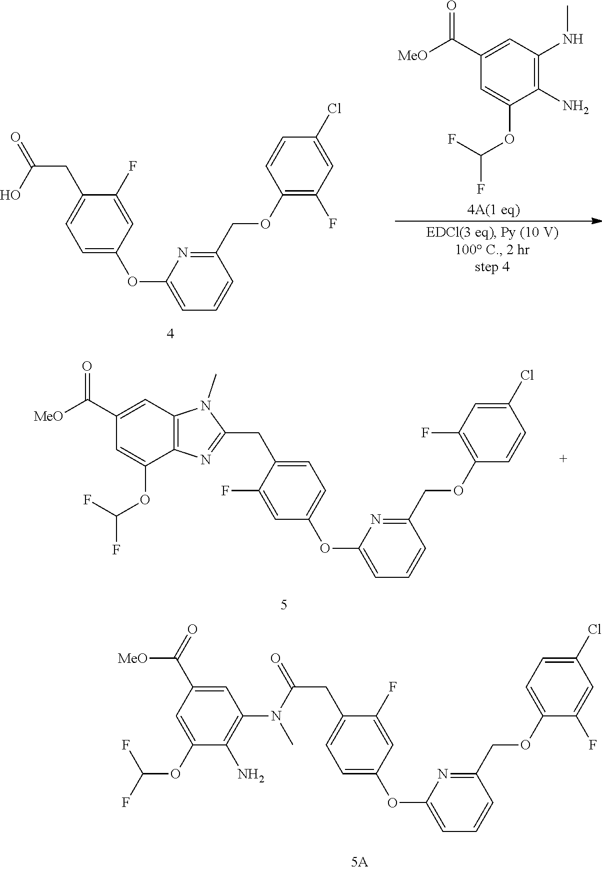 [SMILES: O=C(O)Cc1ccc(Oc2cccc(COc3ccc(Cl)cc3F)n2)cc1F ; CNc1cc(C(=O)OC)cc(OC(F)F)c1N ; COC(=O)c1cc(OC(F)F)c2nc(Cc3ccc(Oc4cccc(COc5ccc(Cl)cc5F)n4)cc3F)n(C)c2c1 ; COC(=O)c1cc(OC(F)F)c(N)c(N(C)C(=O)Cc2ccc(Oc3cccc(COc4ccc(Cl)cc4F)n3)cc2F)c1]

To a solution of 4 (140 mg, 345.02 μmol, 1 eq) in Py (1.5 mL) was added EDCI (198.42 mg, 1.04 mmol, 3 eq) and 4A (84.95 mg, 345.02 μmol, 1 eq). The mixture was stirred at 100° C. for 2 hr. LCMS showed 4 was consumed completely and several new peaks were shown on LCMS and 10% of desired 5 was detected and 56% of desired 5A was detected. The reaction mixture was diluted with H₂O 10 mL and extracted with EtOAc 30 mL (10 mL*3). The combined organic layers were washed with brine 10 mL, dried over Na₂SO₄, filtered, and concentrated under reduced pressure to give a residue. 5&5A (140 mg, crude) was obtained as a purple oil.

LCMS: RT=2.337 min, MS cal.: 615.1/616.1, [M+1]⁺=616.2/617.2

LCMS: RT=2.282 min, MS cal.: 633.1/634.1, [M+1]⁺=634.2/635.2

### General Procedure for Preparation of Intermediate 5:

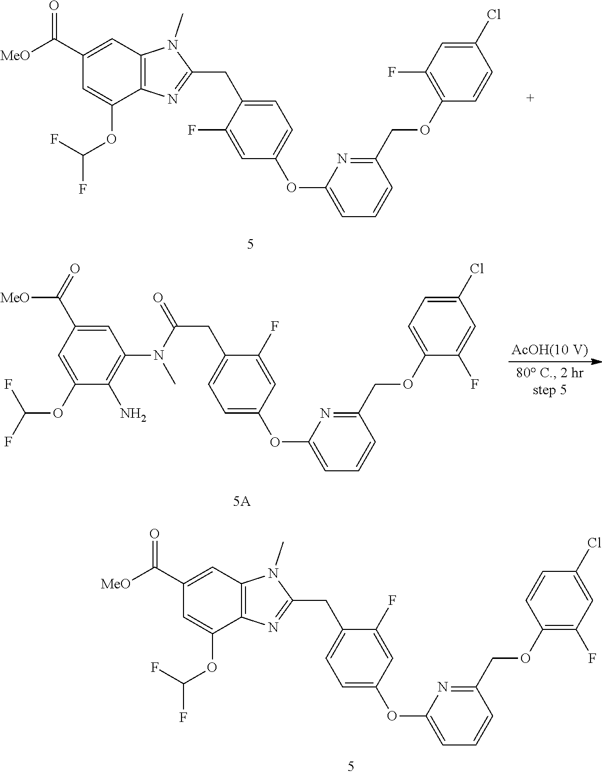 [SMILES: COC(=O)c1cc(OC(F)F)c2nc(Cc3ccc(Oc4cccc(COc5ccc(Cl)cc5F)n4)cc3F)n(C)c2c1 ; COC(=O)c1cc(OC(F)F)c(N)c(N(C)C(=O)Cc2ccc(Oc3cccc(COc4ccc(Cl)cc4F)n3)cc2F)c1 ; COC(=O)c1cc(OC(F)F)c2nc(Cc3ccc(Oc4cccc(COc5ccc(Cl)cc5F)n4)cc3F)n(C)c2c1]

A solution of 5&5A (140 mg, crude) in AcOH (2 mL) was stirred at 80° C. for 2 hr. LCMS showed 5A was consumed completely and one main peak with desired mass was detected. The reaction mixture was adjusted to pH=7 by aq. Na₂CO₃, and then diluted with H₂O 10 mL and extracted with EtOAc 20 mL (10 mL*2). The combined organic layers were washed with brine 10 mL, dried over Na₂SO₄, filtered, and concentrated under reduced pressure to give a residue.

The residue was purified by column chromatography (SiO₂, PE/EtOAc=1/0 to 3/1). 5 (140 mg, crude) was obtained as a yellow oil.

LCMS: RT=1.669 min, MS cal.: 615.1/616.1, [M+1]⁺=616.0/617.0

¹HNMR (400 MHz, MeOH-d4) δ=8.09 (d, J=1.2 Hz, 1H), 7.88-7.83 (m, 1H), 7.67 (s, 1H), 7.46-7.44 (m, 1H), 7.30 (d, J=7.6 Hz, 1H), 7.26 (s, 1H), 7.23 (t, J=8.8 Hz, 1H), 7.16-7.11 (m, 1H), 7.08 (s, 1H), 7.07-7.01 (m, 2H), 7.00-6.96 (m, 1H), 6.94 (d, J=8.4 Hz, 1H), 6.89 (dd, J=2.0, 8.4 Hz, 1H), 5.06 (s, 2H), 4.44-4.41 (m, 2H), 3.95 (s, 3H), 3.85 (s, 3H)

### General Procedure for Preparation of Compound 367 (6):

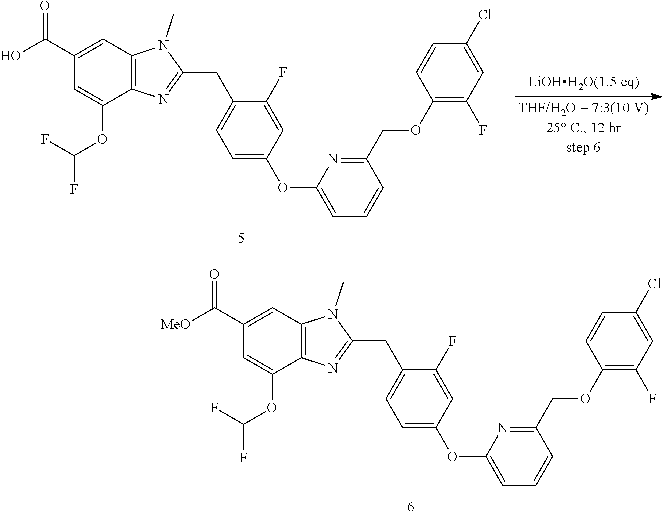 [SMILES: Cn1c(Cc2ccc(Oc3cccc(COc4ccc(Cl)cc4F)n3)cc2F)nc2c(OC(F)F)cc(C(=O)O)cc21 ; COC(=O)c1cc(OC(F)F)c2nc(Cc3ccc(Oc4cccc(COc5ccc(Cl)cc5F)n4)cc3F)n(C)c2c1]

To a solution of 5 (70 mg, 113.64 μmol, 1 eq) in THF (0.7 mL) and H₂O (0.3 mL) was added LiOH·H₂O (7.15 mg, 170.47 μmol, 1.5 eq). The mixture was stirred at 25° C. for 12 hr. LCMS showed 5 was consumed completely and one main peak with desired mass was detected. The mixture was added ACN 0.5 mL. The residue was purified by prep-HPLC (neutral condition: column: Waters Xbridge Prep OBD C18 150*40 mm*10 um; mobile phase: [H₂O (10 mM NH₄HCO₃)-ACN]; gradient: 20%-70% B over 8.0 min). 2-(4-((6-((4-Chloro-2-fluorophenoxy)methyl)pyridin-2-yl)oxy)-2-fluorobenzyl)-4-(difluoromethoxy)-1-methyl-1H-benzo[d]imidazole-6-carboxylic acid (26.38 mg, 42.95 μmol, 37.79% yield) was obtained as a white solid.

LCMS: RT=2.779 min, MS cal.: 601.1/603.1, [M+H]⁺=602.2/604.2

HPLC: RT=14.944 min

¹HNMR (400 MHz, MeOH-d4) δ=8.09-8.07 (m, 1H), 7.86 (t, J=8.0 Hz, 1H), 7.69 (s, 1H), 7.43 (s, 1H), 7.30 (d, J=7.6 Hz, 1H), 7.24 (s, 1H), 7.25-7.20 (m, 1H), 7.14 (dd, J=2.0, 11.2 Hz, 1H), 7.07 (s, 1H), 7.06-7.01 (m, 2H), 7.01-6.96 (m, 1H), 6.94 (d, J=8.4 Hz, 1H), 6.89 (dd, J=2.4, 8.4 Hz, 1H), 5.09-5.06 (m, 2H), 4.42 (s, 2H), 3.85 (s, 3H)

### Example A107—Preparation of Compound 368 and Compound 369. (S)-2-((4-(2-(4-Chloro-2-fluorophenyl)chroman-8-yl)piperidin-1-yl)methyl)-4-(difluoromethoxy)-1-methyl-1H-benzo[d]imidazole-6-carboxylic acid (Compound 368) (10A) & (R)-2-((4-(2-(4-Chloro-2-fluorophenyl)chroman-8-yl)piperidin-1-yl)methyl)-4-(difluoromethoxy)-1-methyl-1H-benzo[d]imidazole-6-carboxylic acid (Compound 369) (10B)

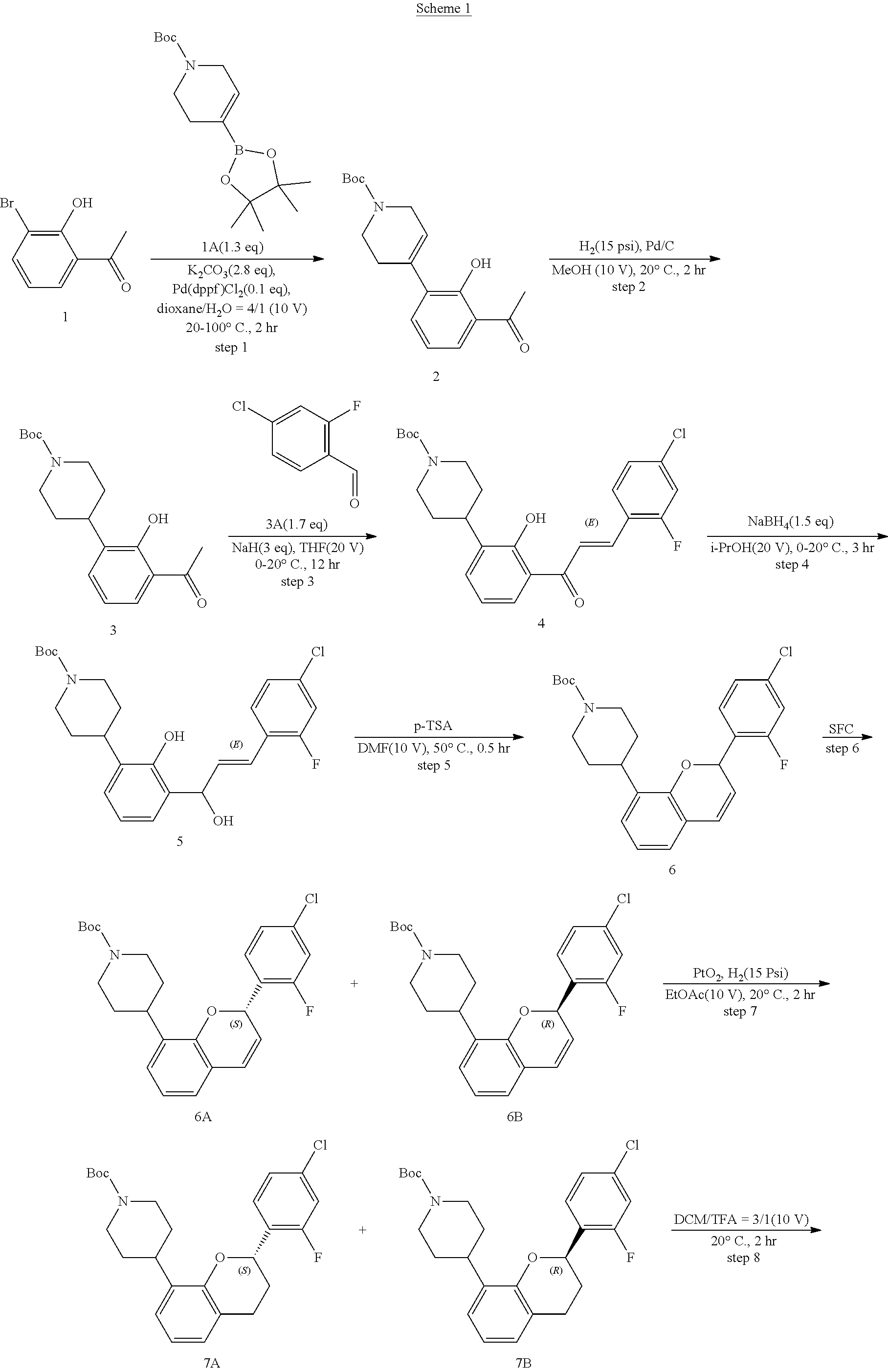

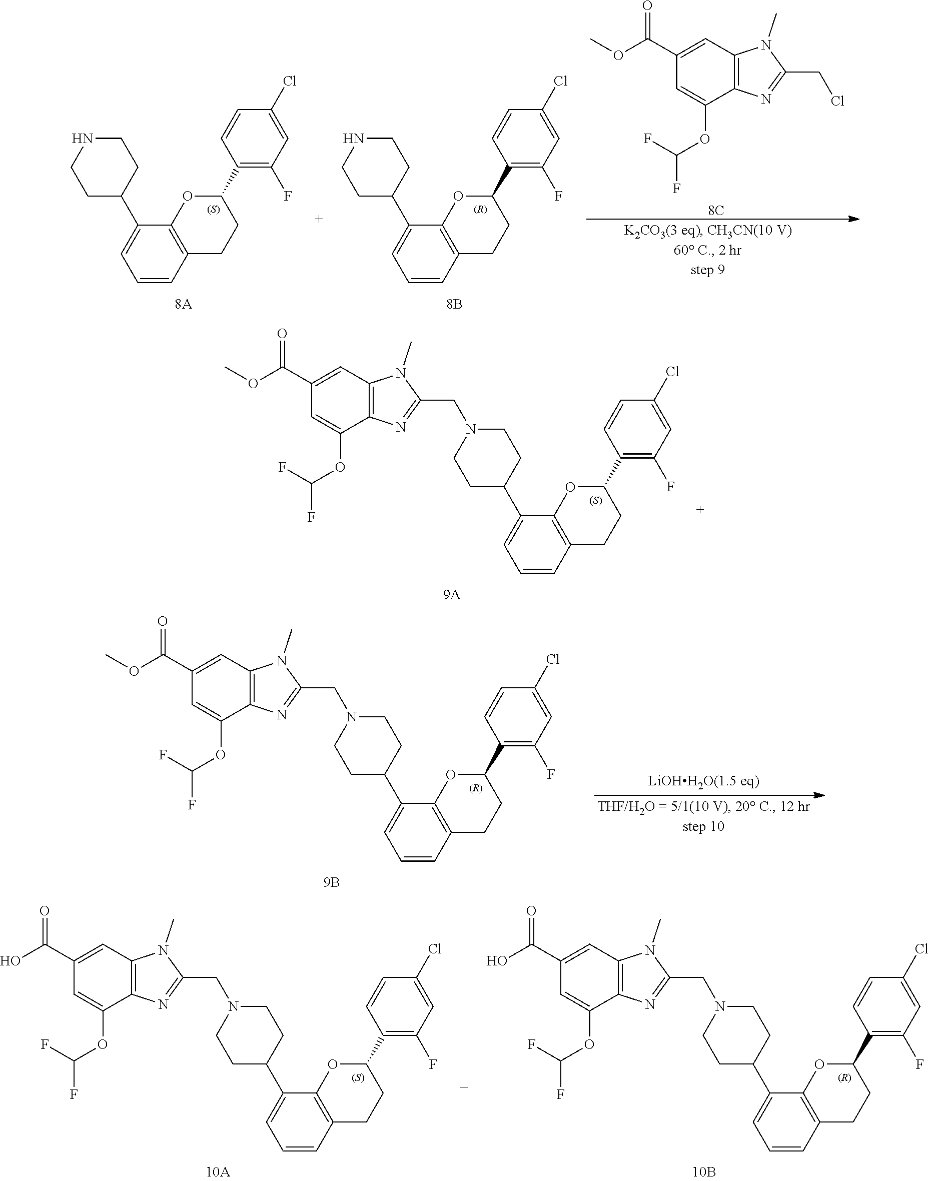

### General Procedure for Preparation of Compound 2:

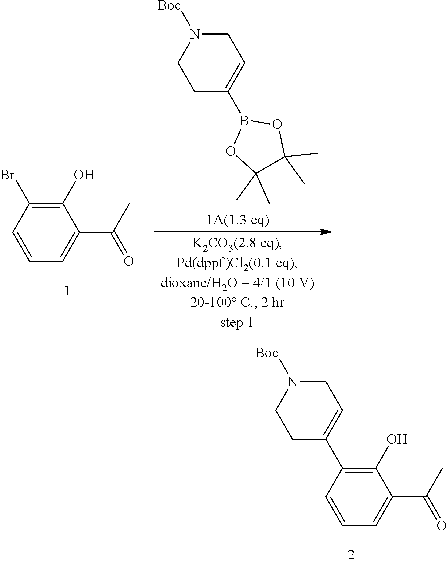 [SMILES: CC(=O)c1cccc(Br)c1O ; CC(C)(C)OC(=O)N1CC=C(B2OC(C)(C)C(C)(C)O2)CC1 ; CC(=O)c1cccc(C2=CCN(C(=O)OC(C)(C)C)CC2)c1O]

To a solution of Compound 1A (7.48 g, 24.18 mmol, 1.3 eq), Compound 1 (4 g, 18.60 mmol, 1 eq) in dioxane (40 mL), H₂O (10 mL) was added K₂CO₃ (7.20 g, 52.08 mmol, 2.8 eq) and Pd(dppf)Cl₂ (1.36 g, 1.86 mmol, 0.1 eq). The mixture was stirred at 100° C. for 2 hr. TLC (Petroleum ether:Ethyl acetate=3:1, R_{f}=0.37) indicated Compound 1 was consumed completely and one new spot formed. Two reactions were combined for workup. The suspension was filtered, and the filter cake was washed with EtOAc (30 mL*3). The combined filtrates were washed with 50 mL H₂O, brine 30 mL, dried over Na₂SO₄, filtered and the filtrate was concentrated under reduced pressure to give a residue. The residue was purified by column chromatography (SiO₂, Petroleum ether:Ethyl acetate=10:1). Compound 2 (10.2 g, 32.14 mmol, 86% yield) was obtained as a yellow oil.

¹H NMR (400 MHz, CHCl3-d) δ=12.77 (br, 1H), 7.68 (dd, J=8.0, 1.6 Hz, 1H), 7.36 (dd, J=7.6, 1.6 Hz, 1H), 6.87 (t, J=7.6 Hz, 1H), 5.87-5.86 (m, 1H), 4.07 (d, J=2.4 Hz, 2H), 3.62 (t, J=5.6 Hz, 2H), 2.65 (s, 3H), 2.54 (d, J=1.6 Hz, 2H), 1.49 (s, 9H)

### General Procedure for Preparation of Compound 3:

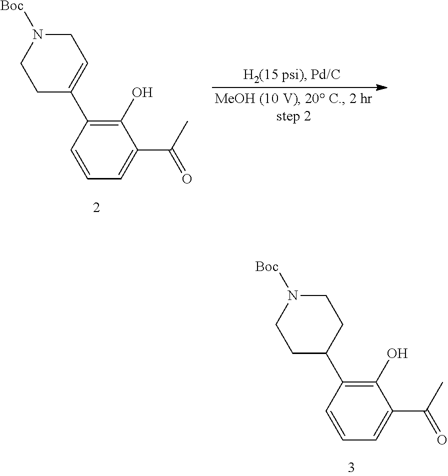 [SMILES: CC(=O)c1cccc(C2=CCN(C(=O)OC(C)(C)C)CC2)c1O ; CC(=O)c1cccc(C2CCN(C(=O)OC(C)(C)C)CC2)c1O]

Equip a 250 mL round bottom flask. At 20° C. (inner temperature) Pd/C (250 mg) in MeOH (50 mL) was added to the reaction mixture at 20° C. under N₂ atmosphere. Then Compound 2 (5 g, 15.75 mmol, 1 eq) in MeOH (50 mL) was added to the mixture at 20° C. After the addition, the suspension was degassed and purged with H₂ for 3 times, and then the mixture was stirred under H₂ (15 Psi) atmosphere at 20° C. for 2 hr. LC-MS showed Compound 2 was consumed completely and one main peak with desired mass was detected. The reaction mixture was filtered through celite pad. The filter cake was rinsed with MeOH (50 mL*3), and the filtrate was concentrated. Compound 3 (5 g, 15.65 mmol, 99% yield) was obtained as a brown oil.

LCMS: RT=1.486 min, MS cal.: 319.2, 320.2, [M−H]⁺=318.1, 319.1

¹H NMR (400 MHz, CHCl3-d) δ=12.75 (s, 1H), 7.63 (dd, J=8.0, 1.6 Hz, 1H), 7.39-7.34 (m, 1H), 6.88 (t, J=8.0 Hz, 1H), 4.25 (d, J=13.2 Hz, 2H), 3.18 (tt, J=12.0, 3.0 Hz, 1H), 2.86 (td, J=13.2, 2.0 Hz, 2H), 2.65 (s, 3H), 1.85 (d, J=12.9 Hz, 2H), 1.58 (td, J=12.0, 4.08 Hz, 2H), 1.49 (s, 9H)

### General Procedure for Preparation of Compound 4:

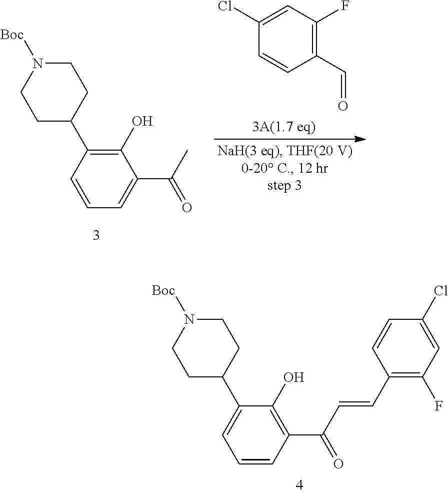 [SMILES: CC(=O)c1cccc(C2CCN(C(=O)OC(C)(C)C)CC2)c1O ; O=Cc1ccc(Cl)cc1F ; CC(C)(C)OC(=O)N1CCC(c2cccc(C(=O)/C=C/c3ccc(Cl)cc3F)c2O)CC1]

Equip a 250 mL three-neck bottle. Then Compound 3 (4.5 g, 14.09 mmol, 1 eq) in THF (45 mL) was added to the mixture at 20° C. At 0° C. (inner temperature), NaH (1.69 g, 42.27 mmol, 60% purity, 3 eq) was added to the reaction mixture at 0° C., the mixture was stirred at 0° C. for 0.5 hr. Then was added Compound 3A (3.80 g, 23.95 mmol, 1.7 eq) in THF (45 mL) at 0° C., the mixture was stirred at 20° C. for 12 hr. LC-MS showed Compound 3 was consumed completely and the desired mass was detected as a main peak. The reaction mixture was quenched by addition NH₄Cl 25 mL at 0° C., and then diluted with H₂O 25 mL and extracted with DCM 75 mL (25 mL*3). The combined organic layers were washed with brine 10 mL, dried over Na₂SO₄, filtered, and concentrated under reduced pressure to give a residue. The residue was purified by column chromatography (SiO₂, Petroleum ether/Ethyl acetate=10/1). Compound 4 (3.5 g, 7.61 mmol, 54% yield) was obtained as a yellow solid.

LCMS: RT=1.891 min, MS cal.: 459.2, 461.2, [M−H]⁺=458.1, 460.1

¹H NMR (400 MHz, DMSO-d6) δ=13.26 (s, 1H), 8.28-8.21 (m, 1H), 8.17 (d, J=16.0 Hz, 1H), 7.91 (d, J=16.0 Hz, 1H), 7.61 (dd, J=10.0, 3.0 Hz, 1H), 7.55 (d, J=7.2 Hz, 1H), 7.47-7.43 (m, 1H), 7.00 (t, J=8.0 Hz, 1H), 4.17-4.03 (m, 2H), 3.16-3.06 (m, 1H), 2.93-2.71 (m, 2H), 1.76 (d, J=12.0 Hz, 2H), 1.58-1.45 (m, 2H), 1.42 (s, 9H)

### General Procedure for Preparation of Compound 5:

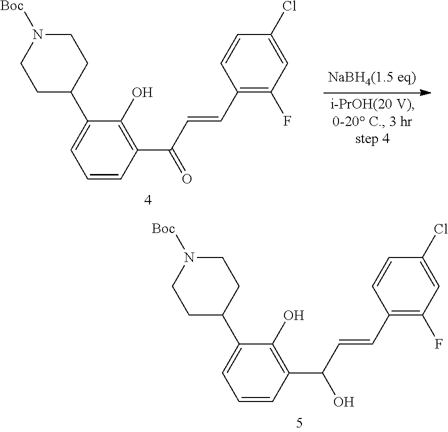 [SMILES: CC(C)(C)OC(=O)N1CCC(c2cccc(C(=O)/C=C/c3ccc(Cl)cc3F)c2O)CC1 ; CC(C)(C)OC(=O)N1CCC(c2cccc(C(O)/C=C/c3ccc(Cl)cc3F)c2O)CC1]

Equip a 250 mL round bottom flask. Then Compound 4 (3.5 g, 7.61 mmol, 1 eq) in i-PrOH (70 mL) was added to the mixture at 20° C. At 0° C. (inner temperature) NaBH₄ (431.84 mg, 11.41 mmol, 1.5 eq) was added dropwise to the mixture at 0° C. under N₂ atmosphere. After the addition, the suspension was degassed and purged with N₂ for 3 times, and then the mixture was stirred at 20° C. for 3 hr. LC-MS showed Compound 4 was consumed completely and the desired mass was detected as a main peak. The reaction mixture was quenched by addition NH₄Cl 5 mL at 0° C., and then diluted with H₂O 10 mL and extracted with EtOAc 15 mL (5 mL*3). The combined organic layers were washed with brine 10 mL, dried over Na₂SO₄, filtered and concentrated under reduced pressure to give a residue. The residue was purified by column chromatography (SiO₂, Petroleum ether/Ethyl acetate=10/1) Compound 5 (2.2 g, 4.76 mmol, 63% yield) was obtained as a white solid.

LCMS: RT=0.643 min, MS cal.: 461.1, 463.1, [M+Na]⁺=484.1, 486.1

¹H NMR (400 MHz, CHCl3-d) δ=7.38 (t, J=8.0 Hz, 1H), 7.16-7.09 (m, 2H), 7.04-6.99 (m, 1H), 6.92-6.84 (m, 2H), 6.61 (dd, J=10.0, 1.6 Hz, 1H), 6.22 (d, J=2.0 Hz, 1H), 5.77 (dd, J=10.0, 4.0 Hz, 1H), 4.28-4.12 (m, 3H), 2.98 (tt, J=12.0, 4.0 Hz, 1H), 2.76 (qd, J=13.0, 2.8 Hz, 2H), 1.88-1.74 (m, 2H), 1.54 (d, J=4.0 Hz, 2H), 1.48-1.43 (m, 9H)

### General Procedure for Preparation of Compound 6:

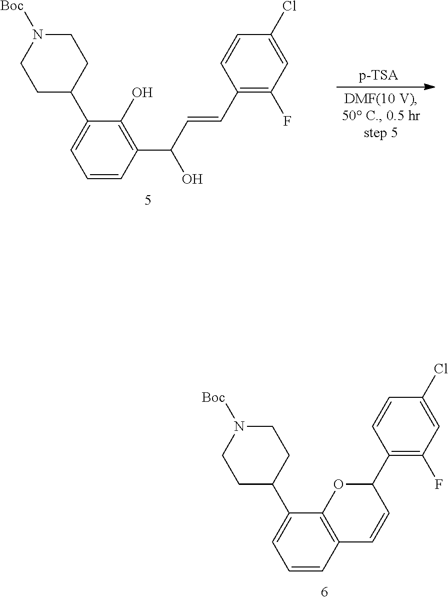 [SMILES: CC(C)(C)OC(=O)N1CCC(c2cccc(C(O)/C=C/c3ccc(Cl)cc3F)c2O)CC1 ; CC(C)(C)OC(=O)N1CCC(c2cccc3c2OC(c2ccc(Cl)cc2F)C=C3)CC1]

To a solution of Compound 5 (1 g, 2.16 mmol, 1 eq) in DMF (10 mL) was added p-TsOH (111.83 mg, 649.42 μmol, 0.3 eq). The mixture was stirred at 50° C. for 0.5 hr. LC-MS showed 5 was consumed completely and the desired mass was detected as a main peak. The mixture was diluted with H₂O (15 mL), extracted with EtOAc (15 mL*3). The combined organic layer was washed with 10 mL brine, dried over Na₂SO₄, filtered and the filtrate was concentrated to give a crude product. The residue was purified by column chromatography (SiO₂, Petroleum ether/Ethyl acetate=10/1) 6 (800 mg, 1.80 mmol, 83% yield) was obtained as a colorless oil.

LCMS: RT=1.815 min, MS cal.: 443.2, 445.2, [M−55]⁺=387.9, 389.9

¹H NMR (400 MHz, CHCl3-d) δ=7.36-7.41 (m, 1H), 7.17-7.09 (m, 2H), 7.02 (dd, J=7.2, 2.0 Hz, 1H), 6.92-6.84 (m, 2H), 6.61 (dd, J=10.0, 2.0 Hz, 1H), 6.22 (dd, J=3.6, 1.20 Hz, 1H), 5.79-5.74 (m, 1H), 4.24-4.16 (m, 2H), 2.98 (tt, J=12.0, 3.4 Hz, 1H), 2.76 (qd, J=30.0, 13.0 Hz, 2H), 1.78 (d, J=13.0 Hz, 1H), 1.54-1.50 (m, 2H), 1.47 (s, 9H)

### General Procedure for Preparation of Intermediate 6A & 6B:

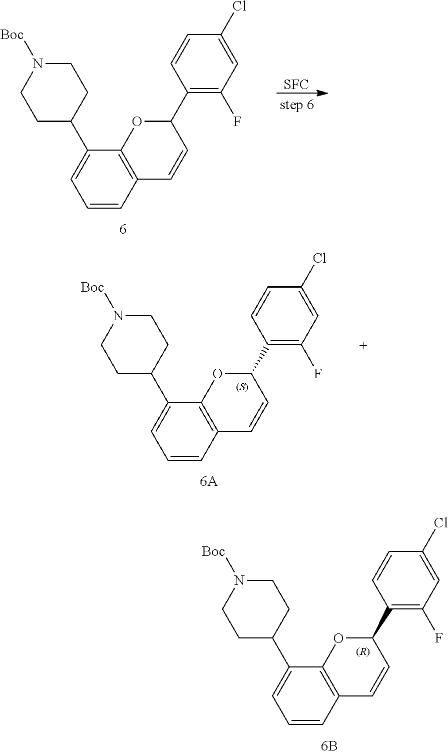

6 (800 mg, 1.80 mmol, 1 eq) was further separated by SFC (column: DAICEL CHIRALCEL OD (250 mm*50 mm, 10 um); mobile phase: [CO₂-MeOH]; B %: 26%, isocratic elution mode). The reaction mixture was concentrated under reduced pressure to remove EtOH. 6A (300 mg, 675.77 μmol, 38% yield) was obtained as a colorless oil. 6B (200 mg, 450.51 μmol, 25% yield) was obtained as a colorless oil.

LCMS: RT=0.729 min, MS cal.: 443.2, 445.2, [M−55]⁺=388.1, 390.1

LCMS: RT=0.719 min, MS cal.: 443.2, 445.2, [M−55]⁺=388.1, 390.0

SFC: ee %=100.00%

SFC: ee %=99.92%

¹H NMR (400 MHz, DMSO-d6) δ=7.52 (dd, J=10.0, 2.0 Hz, 1H), 7.38-7.33 (m, 1H), 7.31-7.27 (m, 1H), 7.03 (d, J=7.6 Hz, 1H), 7.01-6.97 (m, 1H), 6.87-6.82 (m, 1H), 6.74 (dd, J=10.0, 1.2 Hz, 1H), 6.24 (d, J=3.8 Hz, 1H), 5.91 (dd, J=10.0, 4.0 Hz, 1H), 4.07-3.90 (m, 2H), 2.92-2.82 (m, 1H), 2.79-2.59 (m, 2H), 1.67-1.58 (m, 1H), 1.56-1.42 (m, 1H), 1.39 (s, 10H)

¹H NMR (400 MHz, DMSO-d6) δ=7.52 (dd, J=10.0, 2.0 Hz, 1H), 7.40-7.33 (m, 1H), 7.32-7.26 (m, 1H), 7.03 (d, J=7.6 Hz, 1H), 6.99 (dd, J=7.2, 1.2 Hz, 1H), 6.89-6.81 (m, 1H), 6.74 (dd, J=10.0, 1.2 Hz, 1H), 6.24 (d, J=3.8 Hz, 1H), 5.91 (dd, J=10.0, 4.0 Hz, 1H), 4.08-3.92 (m, 2H), 2.97-2.81 (m, 1H), 2.80-2.59 (m, 2H), 1.63 (d, J=13.4 Hz, 1H), 1.53-1.42 (m, 1H), 1.41-1.38 (m, 9H), 1.33 (d, J=4.8 Hz, 2H)

### General Procedure for Preparation of Intermediate 7A:

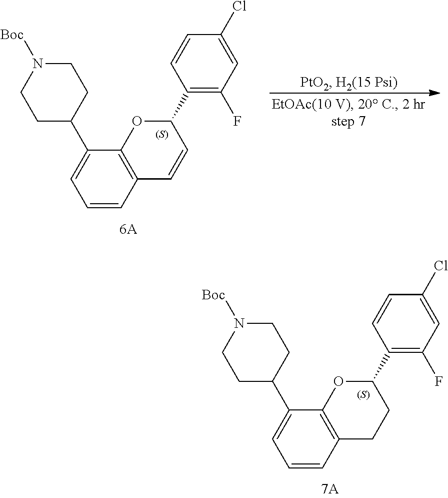

Equip a 50 mL round bottom flask. PtO₂ (10 mg) in EtOAc (2 mL) was added to the reaction mixture at 20° C. under N₂ atmosphere. Then 6A (200.00 mg, 450.51 μmol, 1 eq) in EtOAc (2 mL) was added to the mixture at 20° C. After the addition, the suspension was degassed and purged with H₂ for 3 times, and then the mixture was stirred under H₂ (15 Psi) atmosphere at 20° C. for 2 hr. LC-MS showed 6A was consumed completely and the desired mass was detected as a main peak. The reaction mixture was filtered through celite pad. The filter cake was rinsed with EtOAc 54 mL (18 mL*3), and the filtrate was concentrated. 7A (200 mg, 448.48 μmol, 100% yield) was obtained as a white solid.

LCMS: RT=0.758 min, MS cal.: 565.2, 566.2, [M+H]⁺=566.2, 567.3

¹H NMR (400 MHz, CHCl3-d) δ=7.43 (t, J=8.0 Hz, 1H), 7.19 (dd, J=8.0, 1.6 Hz, 1H), 7.13 (dd, J=10.0, 2.0 Hz, 1H), 7.03 (d, J=7.6 Hz, 1H), 6.98 (d, J=7.0 Hz, 1H), 6.91-6.85 (m, 1H), 5.36-5.31 (m, 1H), 4.26-4.17 (m, 2H), 3.11-2.99 (m, 2H), 2.85-2.72 (m, 3H), 2.30-2.22 (m, 1H), 2.05-1.94 (m, 1H), 1.90-1.75 (m, 2H), 1.67-1.58 (m, 2H), 1.47 (s, 9H)

### General Procedure for Preparation of Intermediate 8A:

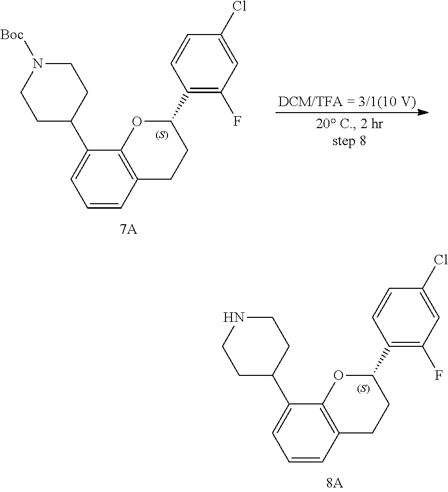

To a solution of 7A (80 mg, 403.63 μmol, 1 eq) in DCM (3 mL) was added TFA (1 mL). The mixture was stirred at 20° C. for 1 hr. LC-MS showed 7A was consumed completely and the desired mass was detected as a main peak. The mixture was poured into aq. Na₂CO₃ 10 mL to adjust pH=12, then diluted with H₂O 10 mL and extracted with DCM 10 mL, the combined organic layers were washed with 10 mL, dried over Na₂SO₄, filtered and concentrated under reduced pressure to give a residue. 8A (135 mg, 390.36 μmol, 97% yield) was obtained as a brown solid.

LCMS: RT=0.457 min, MS cal.: 345.1, 347.1, [M+H]⁺=346.1, 348.0

¹H NMR (400 MHz, CHCl3-d) δ=7.44 (t, J=8.0 Hz, 1H), 7.2 (d, J=8.4 Hz, 1H), 7.13 (dd, J=10.0, 2.0 Hz, 1H), 7.08 (d, J=7.6 Hz, 1H), 6.97 (d, J=7.0 Hz, 1H), 6.92-6.86 (m, 1H), 5.36-5.31 (m, 1H), 3.22 (t, J=12.8 Hz, 2H), 3.13-2.98 (m, 2H), 2.84-2.74 (m, 3H), 2.58-2.46 (m, 1H), 2.30-2.22 (m, 1H), 2.05-1.95 (m, 1H), 1.94-1.80 (m, 2H), 1.75-1.61 (m, 2H)

### General Procedure for Preparation of Intermediate 9A:

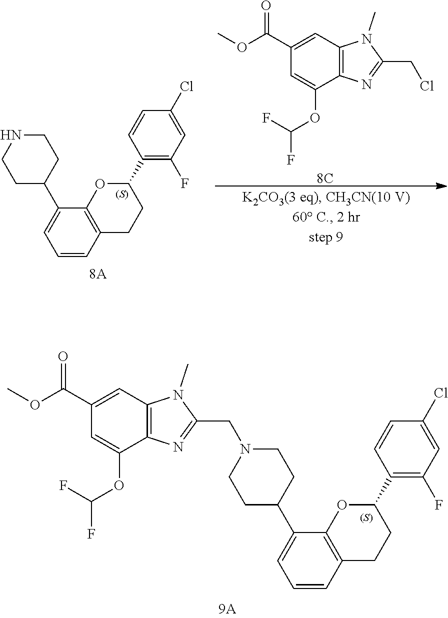

To a solution of 8C (98 mg, 321.65 μmol, 1 eq) 8A (133.49 mg, 385.98 μmol, 1.2 eq) in CH₃CN (1 mL) was added K₂CO₃ (44.45 mg, 321.65 μmol, 1 eq). The mixture was stirred at 60° C. for 2 hr. LC-MS showed 8A was consumed completely and the desired mass was detected as a main peak. The mixture was diluted with H₂O (10 mL), extracted with EtOAc 15 mL (5 mL*3). The combined organic layer was washed with 10 mL brine, dried over Na₂SO₄, filtered and the filtrate was concentrated to give a crude product. The residue was purified by column chromatography (SiO₂, Petroleum ether/Ethyl acetate=3/1) 9A (150 mg, 244.28 μmol, 76% yield) was obtained as a white solid.

LCMS: RT=0.543 min, MS cal.: 613.2, 614.2, [M+H]⁺=614.3, 615.3

¹H NMR (400 MHz, CHCl3-d) δ=8.00 (s, 1H), 7.76-7.67 (m, 1H), 7.42 (t, J=6.8 Hz, 1H), 7.22 (d, J=8.4 Hz, 1H), 7.20 (t, J=72.8 Hz, 1H), 7.15 (d, J=10.0 Hz, 1H), 7.06 (d, J=6.4 Hz, 1H), 7.02-6.95 (m, 1H), 6.91-6.84 (m, 1H), 5.33 (s, 1H), 4.05-3.93 (m, 7H), 3.93-3.81 (m, 2H), 3.08-2.98 (m, 3H), 2.80 (d, J=16.4 Hz, 1H), 2.30-2.19 (m, 2H), 2.08-1.93 (m, 2H), 1.91-1.64 (m, 4H)

### General Procedure for Preparation of Compound 368 (10A):

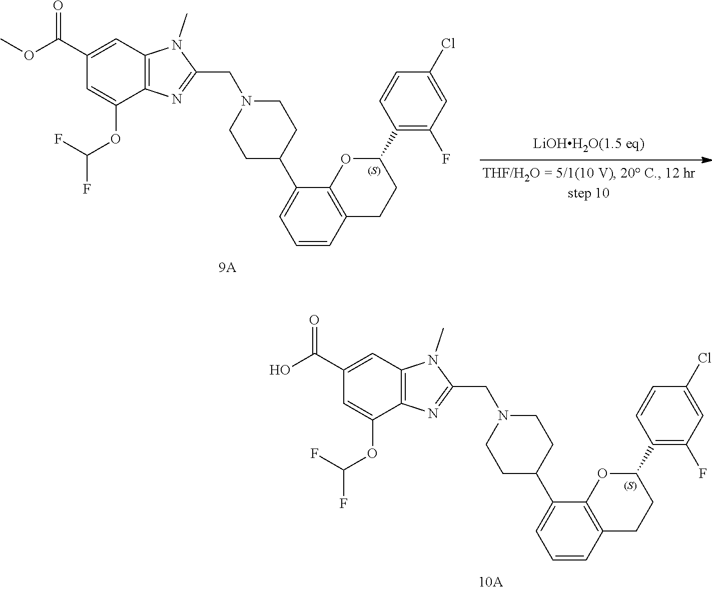

To a solution of 9A (120 mg, 195.42 μmol, 1 eq) in THF (1.5 mL) was added LiOH·H₂O (12.30 mg, 293.13 μmol, 1.5 eq) in H₂O (0.3 mL). The mixture was stirred at 20° C. for 12 hr. LC-MS showed 9A was consumed completely and one main peak with desired mass was detected. The reaction was combined for purification. The residue was purified by prep-HPLC (column: Waters Xbridge Prep OBD C18 150*40 mm*10 um; mobile phase: [H₂O (10 mM NH₄HCO₃)-ACN]; gradient: 30%-75% B over 8.0 min). (S)-2-((4-(2-(4-Chloro-2-fluorophenyl)chroman-8-yl)piperidin-1-yl)methyl)-4-(difluoromethoxy)-1-methyl-1H-benzo[d]imidazole-6-carboxylic acid (22.13 mg, 36.88 μmol, 19% yield) was obtained as a white solid.

LCMS: RT=2.974 min, MS cal.: 599.2, 600.2, [M+H]⁺=600.2, 601.2

¹H NMR (400 MHz, DMSO-d6) δ=8.05 (s, 1H), 7.61 (t, J=76.0 Hz, 1H), 7.56-7.52 (m, 1H), 7.50 (d, J=1.2 Hz, 2H), 7.39 (dd, J=8.0, 2.0 Hz, 1H), 7.01 (d, J=7.2 Hz, 1H), 6.95 (d, J=6.8 Hz, 1H), 6.84-6.78 (m, 1H), 5.33-5.28 (m, 1H), 3.91 (s, 3H), 3.81 (s, 2H), 2.94-3.04 (m, 1H), 2.93-2.86 (m, 2H), 2.85-2.77 (m, 1H), 2.76-2.69 (m, 1H), 2.21-2.08 (m, 3H), 2.00-1.90 (m, 1H), 1.79-1.70 (m, 1H), 1.70-1.53 (m, 3H)

### General Procedure for Preparation of Intermediate 7B:

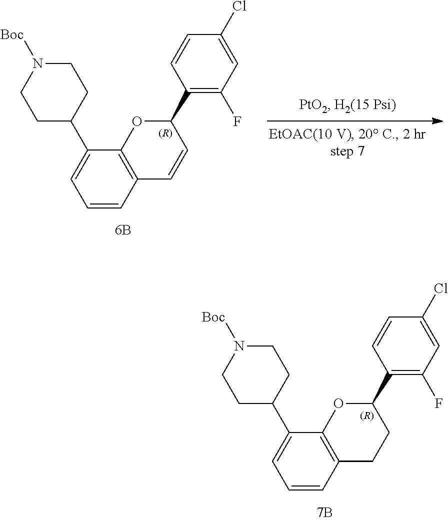

Equip a 50 mL round bottom flask. PtO₂ (10.23 mg) in EtOAc (0.5 mL) was added to the reaction mixture at 20° C. under N₂ atmosphere. Then 6B (0.1 g, 225.26 μmol, 1 eq) in EtOAc (0.5 mL) was added to the mixture at 20° C. After the addition, the suspension was degassed and purged with H₂ for 3 times, and then the mixture was stirred under H₂ (15 psi) atmosphere at 20° C. for 2 hr. LCMS showed 6B was consumed and desired mass was detected. The reaction mixture was filtered through celite pad. The filter cake was rinsed with EtOAc 60 mL (20 mL*3), and the filtrate was concentrated. 7B (80 mg, 179.39 μmol, 80% yield) was obtained as a white solid.

LCMS: RT=0.748 min, MS cal.: 445.2, 447.2, [M−55]⁺=390.1, 392.1

¹H NMR (400 MHz, DMSO-d6) δ=7.56-7.46 (m, 2H), 7.37 (dd, J=8.4, 2.0 Hz, 1H), 6.98 (dd, J=13.2, 7.6 Hz, 2H), 6.86-6.79 (m, 1H), 5.33 (dd, J=10.0, 1.8 Hz, 1H), 3.03-2.90 (m, 2H), 2.81-2.69 (m, 3H), 2.24-2.13 (m, 1H), 1.97-1.90 (m, 1H), 1.79-1.63 (m, 4H), 1.52-1.44 (m, 2H), 1.38 (s, 9H)

### General Procedure for Preparation of Intermediate 8B:

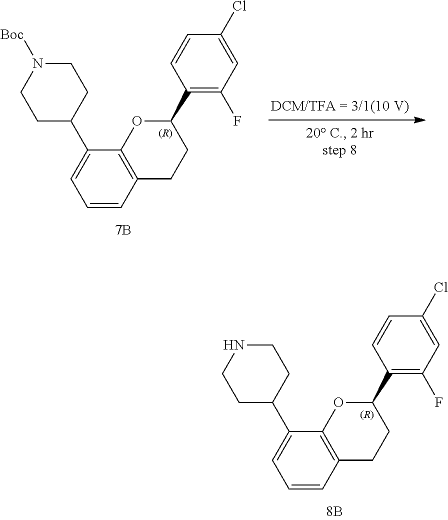

To a solution of 7B (80 mg, 179.39 μmol, 1 eq) was dissolved in DCM (0.6 mL) and TFA (0.2 mL). The reaction mixture was stirred for 2 h at 20° C. LCMS showed 7B was consumed and desired mass was detected. The mixture was concentrated under vacuum. 8B (55 mg, 159.03 μmol, 89% yield) was obtained as a white solid.

LCMS: RT=0.451 min, MS cal.: 345.1, 347.1, [M+H]⁺=346.1, 347.0

¹H NMR (400 MHz, DMSO-d6) δ=7.56-7.47 (m, 2H), 7.38 (dd, J=8.4, 2.0 Hz, 1H), 7.00 (d, J=7.6 Hz, 1H), 6.95 (d, J=6.8 Hz, 1H), 6.87-6.81 (m, 1H), 5.35-5.28 (m, 1H), 3.04-2.90 (m, 4H), 2.70 (br s, 1H), 2.55 (br dd, J=12.0, 2.0 Hz, 1H), 2.22-2.14 (m, 1H), 1.97-1.88 (m, 1H), 1.76-1.59 (m, 4H), 1.48 (ddd, J=15.6, 12.0, 4.0 Hz, 2H)

### General Procedure for Preparation of Intermediate 9B:

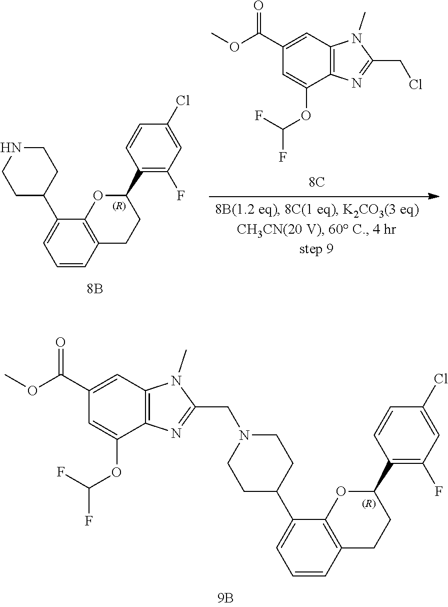

To a solution of 8B (54.48 mg, 157.54 μmol, 1.2 eq) and 8C (40 mg, 131.29 μmol, 1 eq) was dissolved in ACN (0.8 mL) and K₂CO₃ (54.43 mg, 393.86 μmol, 3 eq) was added. The reaction mixture was stirred at 60° C. for 4h. LCMS showed 8B was consumed and desired mass was detected. The residue was diluted with H₂O 3 mL and extracted with EtOAc 30 mL (10 mL*3). The organic phase was separated, washed with brine 15 mL (5 mL*3), with dried over Na₂SO₄, filtered and concentrated under reduced pressure to give a residue. The Compound was used directly to next step. 9B (70 mg, 114.00 μmol, 87% yield) was obtained as a white solid.

LCMS: RT=0.509 min, MS cal.: 613.2, 614.2, [M+H]⁺=614.2, 615.2

SFC: ee %=99.91%

¹H NMR (400 MHz, DMSO-d6) δ=10 (d, J=1.2 Hz, 1H), 7.63 (t, J=74.4 Hz, 1H), 7.55-7.49 (m, 3H), 7.39 (dd, J=8.4, 1.6 Hz, 1H), 7.01 (d, J=7.6 Hz, 1H), 6.95 (d, J=6.8 Hz, 1H), 6.85-6.78 (m, 1H), 5.31 (br d, J=8.4 Hz, 1H), 3.93 (s, 3H), 3.90 (s, 3H), 3.82 (s, 2H), 3.05-2.95 (m, 1H), 2.91 (br d, J=2.4 Hz, 2H), 2.84-2.68 (m, 2H), 2.22-2.10 (m, 3H), 1.98-1.90 (m, 1H), 1.78-1.57 (m, 4H)

### General Procedure for Preparation of Compound 369 (10B):

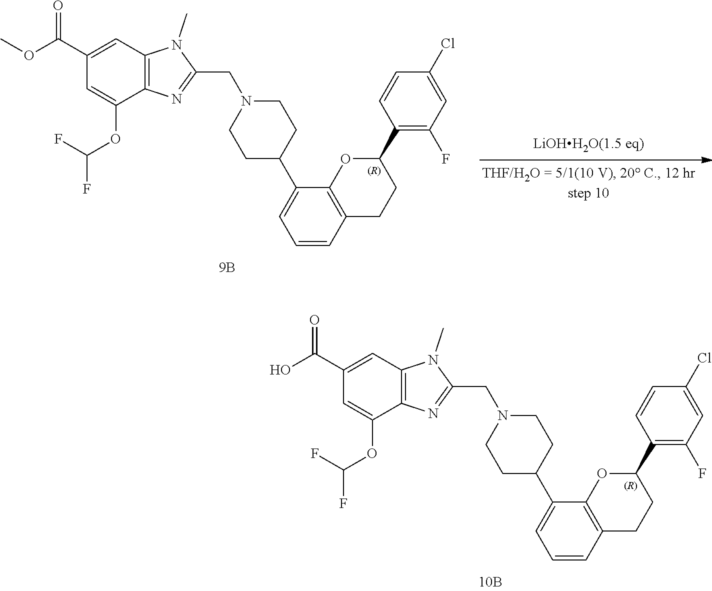

To a solution of 4 (60 mg, 97.71 μmol, 1 eq) was dissolved in THF (0.5 mL) and H₂O (0.1 mL). Then LiOH·H₂O (6.15 mg, 146.57 μmol, 1.5 eq) was added. The reaction mixture was stirred for 12 h at 20° C. LCMS showed 4 was consumed and desired mass was detected. The reaction mixture was partitioned between H₂O 2 mL and EtOAc 4 mL. The organic phase was separated, washed with EtOAc 15 mL (5 mL*3), with dried over Na₂SO₄, filtered and concentrated under reduced pressure to give a residue. The residue was purified by Prep-HPLC (column: Waters Xbridge Prep OBD C18 150*40 mm*10 um; mobile phase: [H₂O (10 mM NH₄HCO₃)-ACN]; gradient: 35%-65% B over 8.0 min). (R)-2-((4-(2-(4-Chloro-2-fluorophenyl)chroman-8-yl)piperidin-1-yl)methyl)-4-(difluoromethoxy)-1-methyl-1H-benzo[d]imidazole-6-carboxylic acid (29.12 mg, 48.53 μmol, 49.67% yield, 100.00% purity) was obtained as a white solid.

LCMS: RT=3.048 min, MS cal.: 599.2/600.2, [M+H]⁺=600.2/601.2

HPLC: RT=13.567 purity: 100.00%

¹H NMR (400 MHz, MeOH-d4) δ=8.05 (s, 1H), 7.62 (t, J=74.4 Hz, 1H), 7.56-7.50 (m, 3H), 7.39 (dd, J=1.8, 8.4 Hz, 1H), 7.01 (d, J=6.8 Hz, 1H), 6.95 (d, J=7.2 Hz, 1H), 6.84-6.79 (m, 1H), 5.31 (br d, J=8.4 Hz, 1H), 3.91 (s, 3H), 3.81 (s, 2H), 2.99 (ddd, J=6.0, 11.2, 16.8 Hz, 1H), 2.94-2.87 (m, 2H), 2.84-2.69 (m, 2H), 2.21-2.10 (m, 3H), 2.01-1.89 (m, 1H), 1.75 (dd, J=1.2, 12.0 Hz, 1H), 1.70-1.55 (m, 3H)

### Example A108—Preparation of Compound 370. 3-(2-((4-(6-((4-Chloro-2-fluorobenzyl)oxy)pyridin-2-yl)piperidin-1-yl)methyl)-4-(difluoromethoxy)-1-methyl-1H-benzo[d]imidazol-6-yl)-1,2,4-thiadiazol-5 (4H)-one (Compound 370) (2)

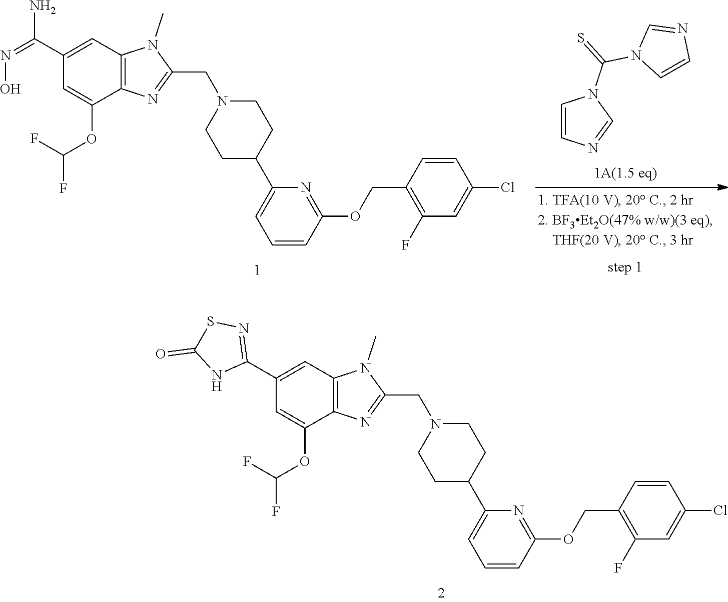 [SMILES: Cn1c(CN2CCC(c3cccc(OCc4ccc(Cl)cc4F)n3)CC2)nc2c(OC(F)F)cc(/C(N)=N\O)cc21 ; S=C(n1ccnc1)n1ccnc1 ; Cn1c(CN2CCC(c3cccc(OCc4ccc(Cl)cc4F)n3)CC2)nc2c(OC(F)F)cc(-c3nsc(=O)[nH]3)cc21]

A mixture of 1 (100 mg, 169.78 μmol, 1 eq), 1A (45.38 mg, 254.67 μmol, 1.5 eq) in THF (1 mL) was degassed and purged with N₂ for 3 times, and then the mixture was stirred at 20° C. for 2 hr under N₂ atmosphere. LCMS (product: RT=1.319 min) showed the 1 was consumed completely. The reaction mixture was quenched by addition H₂O 2 mL at 20° C. and extracted with EtOAc 3 mL*3. The combined organic layers were washed with brine 4 mL*2, dried over Na₂SO₄, filtered, and concentrated under reduced pressure to give a residue. The residue (100 mg, crude) was obtained as a yellow solid.

The residue (100 mg, crude) was added THF (2 mL) at 20° C., then BF₃·Et₂O (287.11 mg, 950.77 μmol, 248.80 μL, 47% purity, 3 eq) was degassed and purged with N₂ for 3 times, and then the mixture was stirred at 20° C. for 3 hr under N₂ atmosphere. LCMS (ET64759-627-P1A, product: RT=1.332 min) showed the 1 was completely. The reaction mixture was quenched by addition H₂O 2 mL at 20° C. and extracted with EtOAc 3 mL*3. The combined organic layers were washed with brine 4 mL*2, dried over Na₂SO₄, filtered, and concentrated under reduced pressure to give a residue. The residue purified by prep-HPLC (column: Waters Xbridge Prep OBD C18 150*40 mm*10 um; mobile phase: [H₂O (10 mM NH₄HCO₃)-ACN]; gradient: 35%-65% B over 8.0 min). 3-(2-((4-(6-((4-Chloro-2-fluorobenzyl)oxy)pyridin-2-yl)piperidin-1-yl)methyl)-4-(difluoromethoxy)-1-methyl-1H-benzo[d]imidazol-6-yl)-1,2,4-thiadiazol-5 (4H)-one (33.87 mg, 51.60 μmol, 16.28% yield, 96.14% purity) was obtained as a white solid.

LCMS: RT=2.977 min, MS cal.: 630.1, [M+H]⁺=631.2

HPLC: RT=13.026 min

¹H NMR (400 MHz, DMSO-d6) δ=8.10 (s, 1H) 7.66-7.60 (m, 1H), 7.65 (t, J=74.4 Hz, 1H) 7.60 (s, 1H), 7.58-7.53 (m, 1H), 7.48-7.42 (m, 1H), 7.28 (d, J=8.0 Hz, 1H), 6.87 (d, J=7.2 Hz, 1H), 6.67 (d, J=8.4 Hz, 1H), 5.36 (s, 2H), 3.95 (s, 3H), 3.89 (s, 2H), 3.01-2.92 (m, 2H), 2.63-2.56 (m, 1H), 2.25 (t, J=11.2 Hz, 2H), 1.84-1.66 (m, 4H)

### Example A109—Preparation of Compound 371. 2-(((1r,4r)-4-(6-((4-Chloro-2-fluorobenzyl)oxy)pyridin-2-yl)cyclohexyl)amino)-4-(difluoromethoxy)-1-methyl-1H-benzo[d]imidazole-6-carboxylic acid (Compound 371) (6)

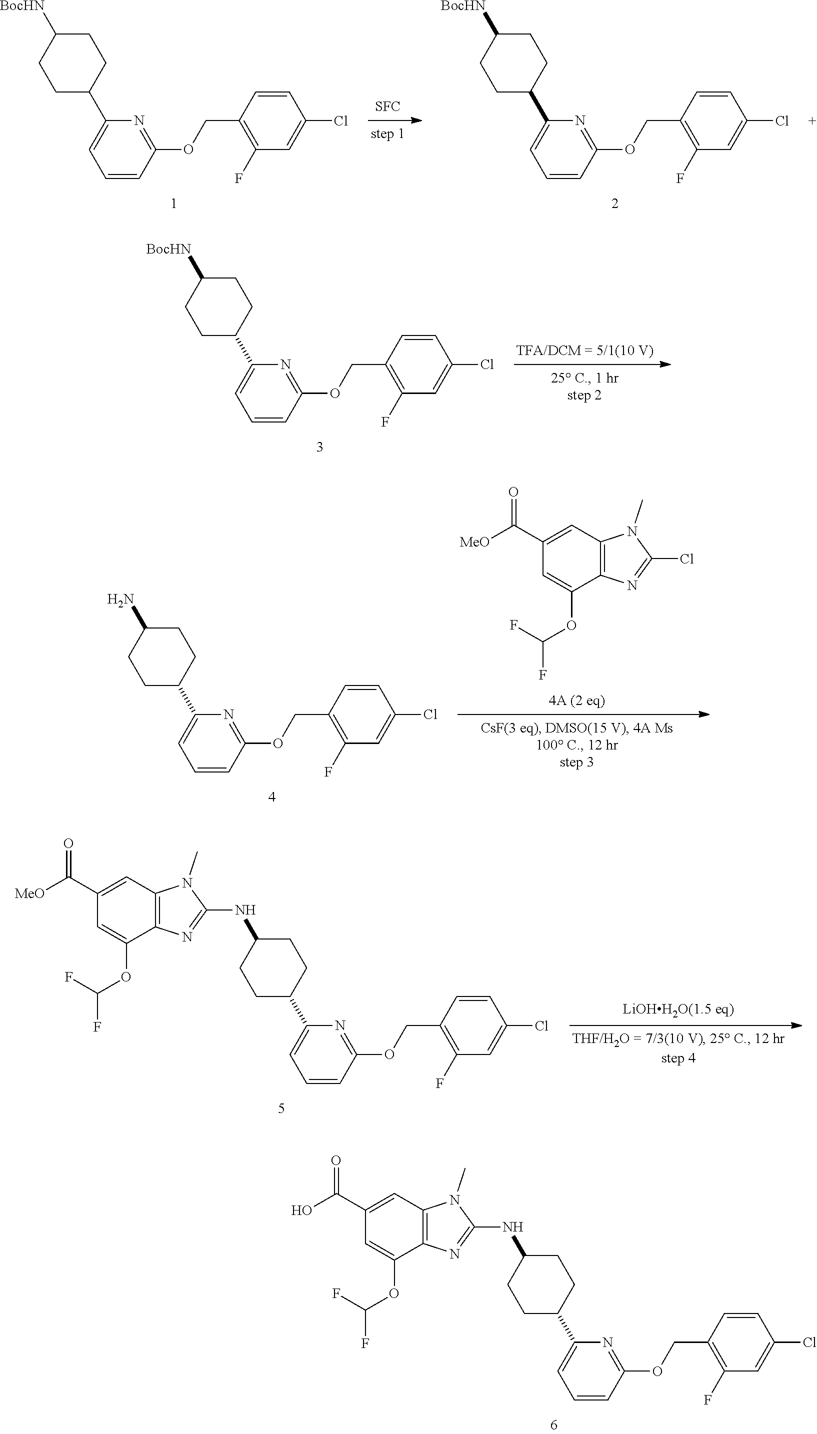

### General Procedure for Preparation of Intermediate 3:

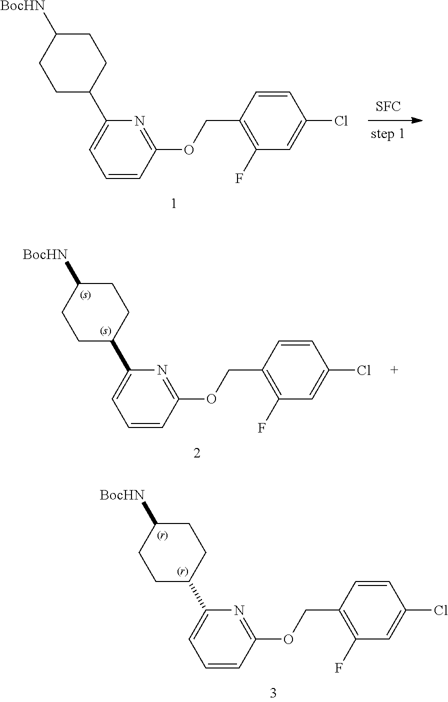

1 was purified by column chromatography (SiO₂, PE/EtOAc=10/1 to 0/1). The residue was further separated by SFC. 3 (0.74 g, 37.00% yield) was obtained as a white oil.

¹H NMR (400 MHz, CHCl3-d) δ=7.52-7.48 (m, 1H), 7.48-7.41 (m, 1H), 7.16-7.10 (m, 2H), 6.72 (d, 1H), 6.60 (d, 1H), 5.41 (s, 2H), 4.43 (s, 1H), 3.52 (s, 1H), 2.61-2.52 (m, 1H), 2.17-2.10 (m, 2H), 2.00-1.93 (m, 2H), 1.73-1.61 (m, 2H), 1.56 (s, 4H), 1.50-1.45 (m, 9H), 1.32-1.19 (m, 2H)

2 (1.1 g, 2.53 mmol, 55.00% yield) was obtained as a white oil.

¹H NMR (400 MHz, MeOH-d4) δ=7.64-7.49 (m, 2H), 7.29-7.14 (m, 2H), 6.89-6.60 (m, 2H), 6.54-6.40 (m, 1H), 5.46-5.36 (m, 2H), 3.76-3.64 (m, 1H), 2.75-2.61 (m, 1H), 1.96-1.75 (m, 4H), 1.72-1.56 (m, 4H), 1.51-1.43 (m, 9H).

### General Procedure for Preparation of Intermediate 4:

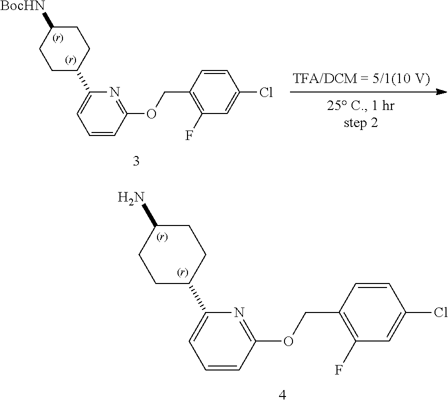

To a solution of 4 (340 mg, 781.73 μmol, 1 eq) in DCM (3.4 mL) was added TFA (1.04 g, 9.15 mmol, 680.00 μL, 11.71 eq). The mixture was stirred at 25° C. for 1 hr. LC-MS (Rt=0.421 min) showed 4 was consumed completely and one main peak with desired mass was detected. The mixture was added with NaHCO₃(aq.) to under pH=10-11. The mixture was diluted with H₂O 5 mL and extracted with DCM 30 mL (10 mL*3). The combined organic layers were washed with brine 5 mL, dried over Na₂SO₄, filtered and concentrated under reduced pressure to give 4 (200 mg, 597.35 μmol, 76.41% yield) was obtained as a green oil.

LCMS: Rt=0.407 min, MS cal.: 334.12/336.12, [M+H]⁺=335.10/337.10

¹H NMR (400 MHz, CHCl3-d) δ=7.50 (t, J=8.0 Hz, 1H), 7.43 (t, J=8.0 Hz, 1H), 7.15-7.09 (m, 2H), 6.71 (d, J=7.2 Hz, 1H), 6.61 (d, J=8.0 Hz, 1H), 5.32 (s, 2H), 3.15-3.02 (m, 1H), 2.68-2.51 (m, 1H), 2.19 (br d, J=11.2 Hz, 2H), 2.07-1.96 (m, 2H), 1.73-1.59 (m, 2H), 1.59-1.46 (m, 2H).

### General Procedure for Preparation of Intermediate 5:

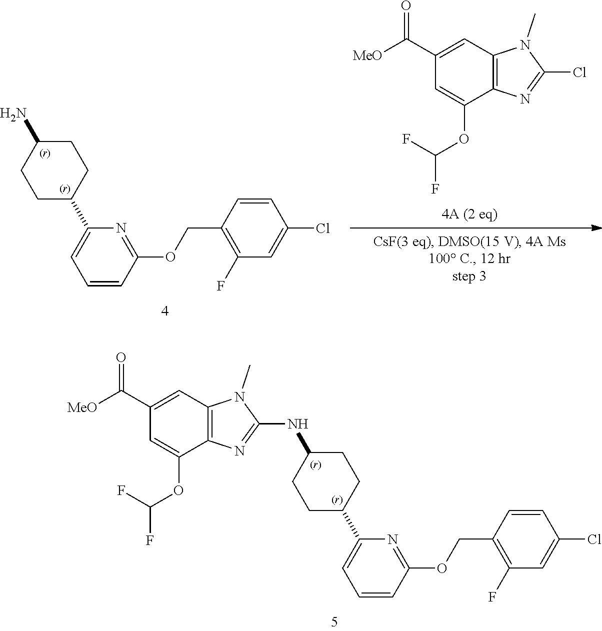

To a solution of 4 (100 mg, 298.67 μmol, 1 eq) and 4A (173.62 mg, 597.35 μmol, 2 eq) in DMSO (1.5 mL) was added CsF (136.11 mg, 896.02 μmol, 3 eq) and molecular sieves (300 mg).

The mixture was stirred at 100° C. for 12 hr. LC-MS (ET77627-307-P1J2, Rt=1.742 min) showed 4 was consumed completely and one main peak with desired mass was detected. The reaction mixture was filtered with EtOAc 10 ml and the filtrate was concentrated. The residue was diluted with H₂O 10 mL and extracted with EtOAc 30 mL (10 mL*3). The combined organic layers were washed with brine 20 mL, dried over Na₂SO₄, filtered, and concentrated under reduced pressure to give a residue. The residue was purified by column chromatography (SiO₂, PE/EtOAc=10/1 to 0/1). 5 (0.08 g, 135.82 μmol, 45.48% yield) was obtained as a yellow oil.

LCMS: Rt=1.742 min, MS cal.: 588.18/590.17, [M+H]⁺=589.1/590.1

LCMS: Rt=1.742 min, MS cal.: 588.18/590.17, [M+H]⁺=589.1/590.1

### General Procedure for Preparation of Compound 371 (6):

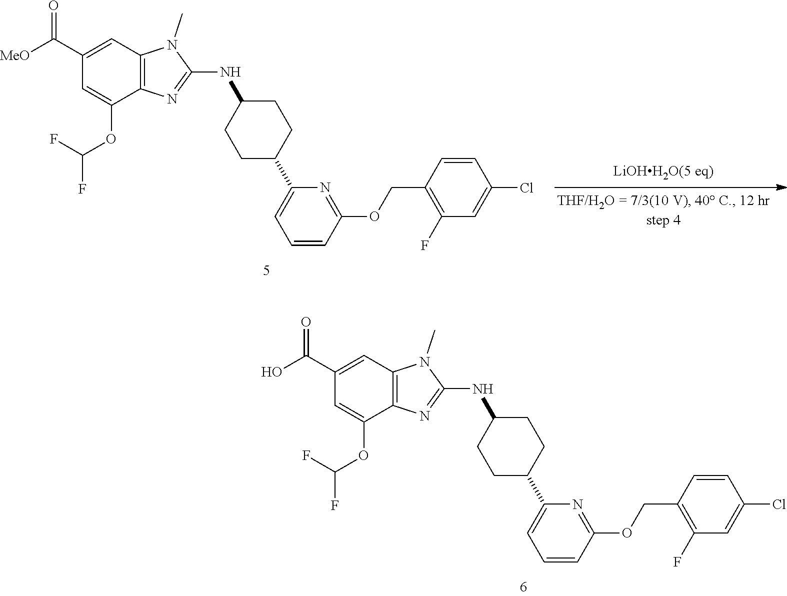

To a solution of 5 (70 mg, 118.84 μmol, 1 eq) in THF (0.7 mL) was added LiOH·H₂O (7.48 mg, 178.27 μmol, 1.5 eq) in H₂O (0.3 mL). The mixture was stirred at 25° C. for 12 h. After monitoring, the reaction was not completely. Then was added LiOH·H₂O (4.99 mg, 118.84 μmol, 1 eq). The mixture was stirred at 25° C. for 2 hr. After monitoring, the reaction was not completely. Then was added LiOH·H₂O (4.99 mg, 118.84 μmol, 1 eq). The mixture was stirred at 25° C. for 12 hr. After monitoring, the reaction was not completely. Then was added LiOH·H₂O (2.49 mg, 59.42 μmol, 0.5 eq). The mixture was stirred at 30° C. for 2 hr. After monitored, the reaction was not completely. Then was added LiOH·H₂O (4.99 mg, 118.84 μmol, 1 eq). The mixture was stirred at 40° C. for 8 hr. LC-MS (Rt=1.265 min) showed 5 was consumed completely and one main peak with desired mass was detected. The reaction was purified directly. The residue was purified by prep-HPLC (column: Waters Xbridge BEH C18 100*30 mm*10 um; mobile phase: [H₂O (10 mM NH₄HCO₃)-can]; gradient: 40%-70% B over 8.0 min) to provide 2-(((1r,4r)-4-(6-((4-chloro-2-fluorobenzyl)o648yridinedin-2-yl)cyclohexyl)amino)-4-(difluoromethoxy)-1-methyl-1H-benzo[d]imidazole-6-carboxylic acid.

LCMS: Rt=1.265 min, MS cal.: 574.16/576.16, [M+H]⁺=575.10/577.0

HPLC: Rt=13.484 min, purity: 100.00%

LCMS: Rt=2.909 min, MS cal.: 574.16/576.16, [M+H]⁺=575.2/577.2

¹H NMR (400 MHz, DMSO-d6) δ=7.-7-7.57 (m, 3H), 7.55 (t, J=75.2 Hz, 1H), 7.-0-7.46 (m, 1H), 7.38 (s, 1H), 7.32 (dd, J=8.4, 2.0 Hz, 1H), 6.-8-6.90 (m, 2H), 6.68 (d, J=8.0 Hz, 1H), 5.40 (s, 2H), 3.-4-3.85 (m, 1H), 3.57 (s, 3H), 2.-6-2.58 (m, 1H), 2.15 (br d, J=11.2 Hz, 2H), 1.93 (br d, J=12.4 Hz, 2H), 1.-4-1.64 (m, 2H), 1.-8-1.47 (m, 2H)

### Example A110—Preparation of Compound 372. 2-(((1r,4r)-4-(6-((4-Chloro-2-fluorobenzyl)o648yridinedin-2-yl)cyclohexyl)(methyl-d3)amino)-4-(difluoromethoxy)-1-methyl-1H-benzo[d]imidazole-6-carboxylic acid (Compound 372) (10)

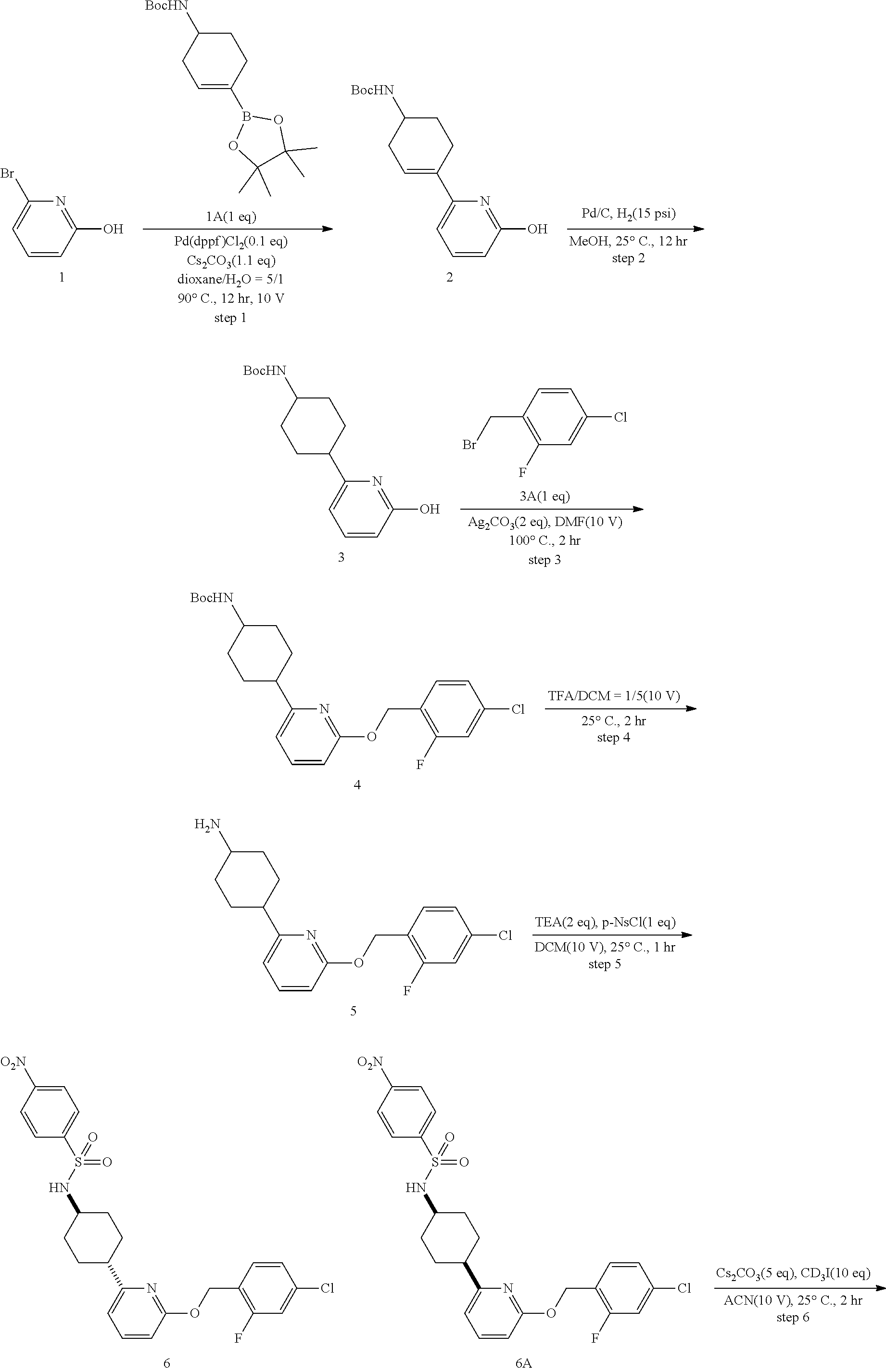

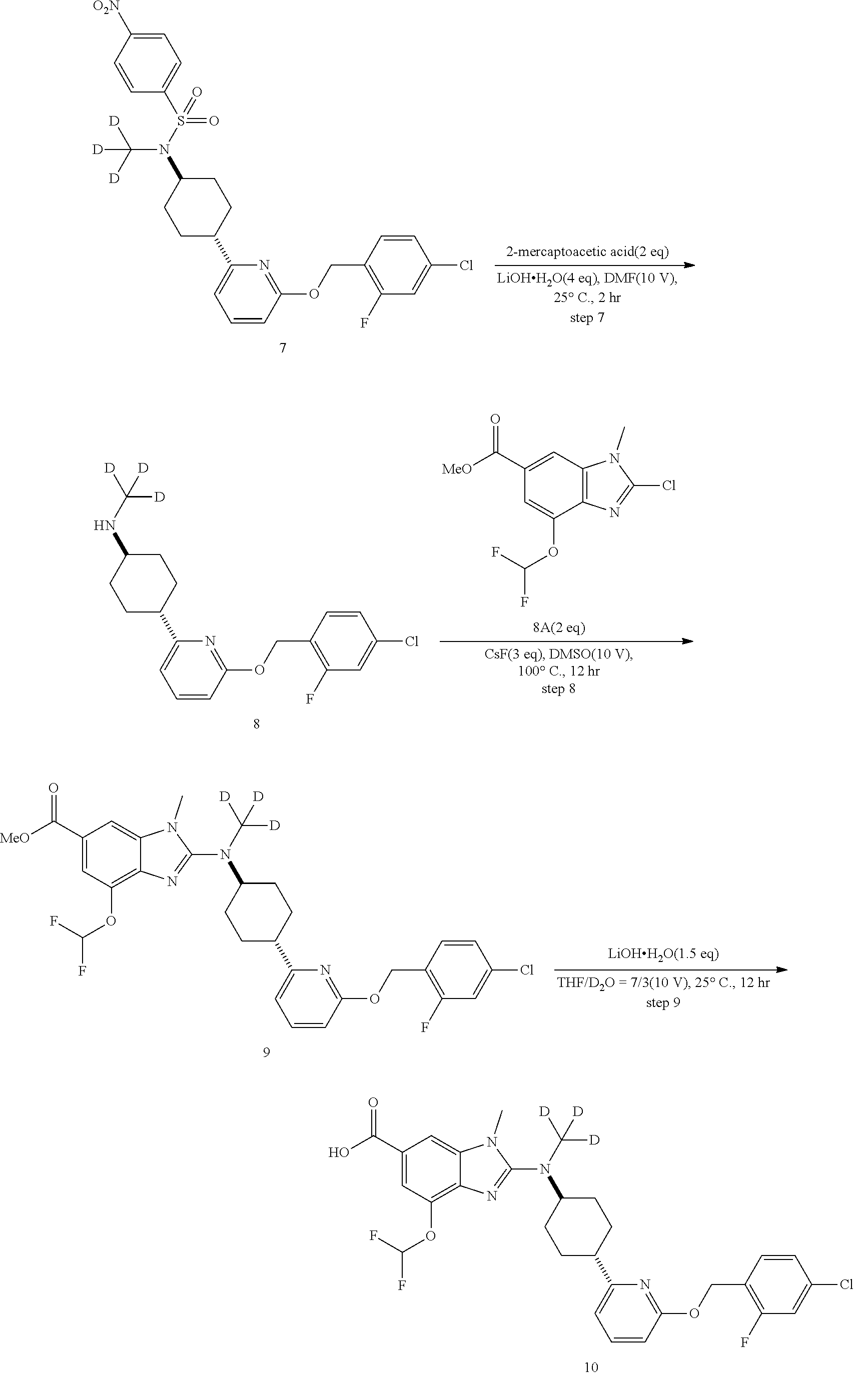

### General Procedure for Preparation of Intermediate 2:

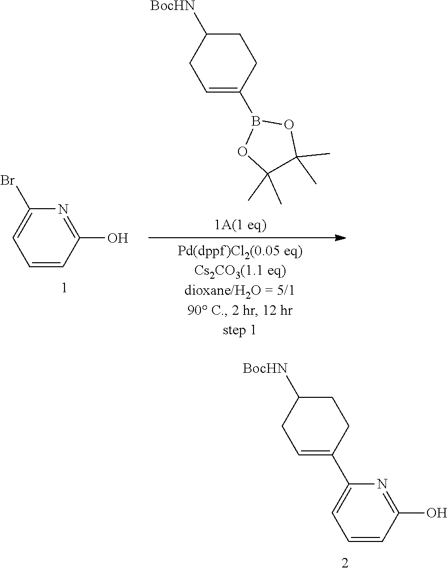 [SMILES: Oc1cccc(Br)n1 ; CC(C)(C)OC(=O)NC1CC=C(B2OC(C)(C)C(C)(C)O2)CC1 ; CC(C)(C)OC(=O)NC1CC=C(c2cccc(O)n2)CC1]

To a solution of 1 (10 g, 57.47 mmol, 1.2 eq) and 1A (15.48 g, 47.89 mmol, 1 eq) in dioxane 5 (100 mL) and H₂O (20 mL) was added Cs₂CO₃ (17.17 g, 52.68 mmol, 1.1 eq) and Pd(dppf)Cl₂· CH₂Cl₂ (1.96 g, 2.39 mmol, 0.05 eq). The mixture was stirred at 90° C. for 12 hr. LC-MS showed 1 was consumed completely and one main peak with desired mass was detected. The reaction mixture was cooled to 25° C., filtered and the filter cake was washed with EtOAc (20 mL*3). The combined filtrate was diluted with H₂O 150 mL, extracted with EtOAc 300 mL (100 mL*3). The combined organic layers were washed with brine 100 mL (50 mL*2), dried over Na₂SO₄, filtered, and concentrated under reduced pressure to give a residue. The residue was purified by column chromatography (SiO₂, Petroleum ether/Ethyl acetate=1/1 to 0/1). 2 (11 g, 37.88 mmol, 79.10% yield) was obtained as a light yellow solid.

¹H NMR (400 MHz, DMSO-d6) δ=11.17 (br s, 1H), 7.38 (dd, J=7.2, 9.2 Hz, 1H), 6.83 (br d, J=7.6 Hz, 1H), 6.35 (br s, 1H), 6.25-6.16 (m, 2H), 3.48 (br s, 1H), 2.37 (br s, 3H), 2.10-1.99 (m, 1H), 1.90-1.80 (m, 1H), 1.55-1.43 (m, 1H), 1.39 (s, 9H)

### General Procedure for Preparation of Intermediate 3:

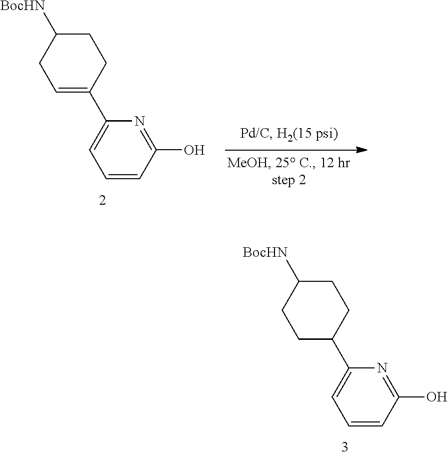 [SMILES: CC(C)(C)OC(=O)NC1CC=C(c2cccc(O)n2)CC1 ; CC(C)(C)OC(=O)NC1CCC(c2cccc(O)n2)CC1]

To a solution of 2 (20 g, 34.44 mmol, 1 eq) in MeOH (200 mL) was added Pd/C (6 g) at 25° C. under H₂ atmosphere. After the addition, purged with H₂ for 3 times, the mixture was stirred at 25° C. for 12 hr under H₂ (15 Psi). TLC showed 2 was consumed completely. The reaction mixture was diluted with MeOH (50 mL), and then filtered through celite pad. The filter cake was rinsed with MeOH (300 mL*3), and the filtrate was concentrated to give 3 (19.9 g, 68.06 mmol, 98.81% yield) as a yellow solid.

LCMS: RT=0.398 min, MS cal.: 292.18, [M−H]⁻=293.1

¹H NMR (400 MHz, CHCl3-d) δ=11.58-11.23 (m, 1H), 7.64-7.45 (m, 1H), 7.35 (td, J=6.4, 8.8 Hz, 1H), 6.99-6.74 (m, 1H), 6.18-6.11 (m, 1H), 6.02 (br dd, J=6.4, 19.2 Hz, 1H), 3.78 (d, J=2.0 Hz, 1H), 3.30-3.20 (m, 1H), 2.46-2.25 (m, 1H), 1.88-1.71 (m, 3H), 1.53 (d, J=13.2 Hz, 4H), 1.41 (d, J=12.0 Hz, 9H), 1.22 (d, J=10.8 Hz, 1H)

### General Procedure for Preparation of Intermediate 4:

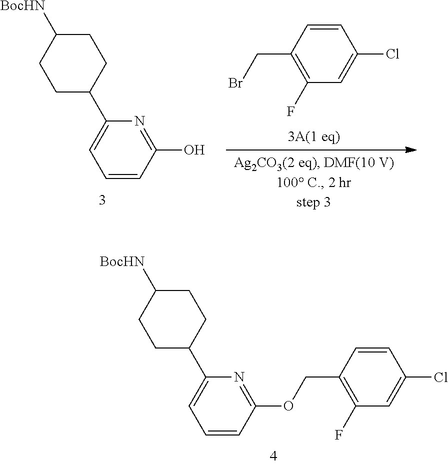 [SMILES: CC(C)(C)OC(=O)NC1CCC(c2cccc(O)n2)CC1 ; Fc1cc(Cl)ccc1CBr ; CC(C)(C)OC(=O)NC1CCC(c2cccc(OCc3ccc(Cl)cc3F)n2)CC1]

To a solution of 3 (17.8 g, 30.44 mmol, 1 eq) in DMF (178 mL) was added Ag₂CO₃ (33.58 g, 60.88 mmol, 2.76 mL, 2 eq) and Compound 3A (13.6 g, 30.44 mmol, 1 eq) at 25° C. Then the mixture was stirred at 25° C. for 2 hr. TLC showed 3 was consumed completely. The reaction mixture was filtered, and the filter cake was washed by EtOAc (100 mL*3), then the combined filtrate was added H₂O 400 mL and extracted with EtOAc 30 mL (100 mL*3). The combined organic layers were washed with brine 400 mL (200 mL*2), dried over Na₂SO₄, filtered, and concentrated under reduced pressure to give a residue. The residue was purified by column chromatography (SiO₂, Petroleum ether/Ethyl acetate=1/0 to 0/1). Compound 4 (20 g, 45.98 mmol, 75.53% yield) was obtained as a white solid.

LCMS: RT=0.685 min, MS cal.: 434.18, [M+H]⁻=435.1

¹H NMR (400 MHz, CHCl3-d) δ=7.42 (t, J=7.6 Hz, 1H), 7.37 (t, J=8.0 Hz, 1H), 7.06-7.01 (m, 2H), 6.64 (d, J=7.6 Hz, 1H), 6.52 (d, J=8.0 Hz, 1H), 5.33 (s, 2H), 4.44-4.29 (m, 1H), 3.57-3.30 (m, 1H), 2.55-2.39 (m, 1H), 2.06 (d, J=10.0 Hz, 2H), 1.93-1.86 (m, 2H), 1.61-1.53 (m, 3H), 1.39 (s, 9H), 1.17 (dd, J=2.8, 12.4 Hz, 1H)

### General Procedure for Preparation of Intermediate 5:

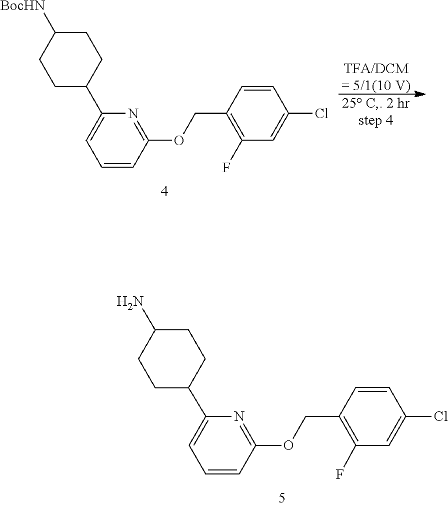 [SMILES: CC(C)(C)OC(=O)NC1CCC(c2cccc(OCc3ccc(Cl)cc3F)n2)CC1 ; NC1CCC(c2cccc(OCc3ccc(Cl)cc3F)n2)CC1]

To a solution of 4 (7 g, 16.09 mmol, 1 eq) in DCM (60 mL) was added TFA (10 mL) at 25° C. The mixture was stirred at 25° C. for 2 hr. The mixture was stirred at 25° C. for 2 hr. TLC showed 4 was consumed completely. The reaction mixture was adjusted to pH 8 with Na₂CO₃ aq. The aqueous layer was extracted with DCM (50 mL*3). The combined organic phase was washed with brine (50 mL*2), dried with anhydrous Na₂SO₄, filtered, and concentrated in vacuum. 5 (5 g, 14.93 mmol, 92.79% yield) was obtained as a yellow solid.

¹H NMR (400 MHz, CHCl3-d) δ=7.42 (t, J=8.0 Hz, 1H), 7.38-7.28 (m, 1H), 7.08-7.00 (m, 2H), 6.71 (d, J=7.2 Hz, 1H), 6.52 (d, J=8.0 Hz, 1H), 5.38-5.29 (m, 2H), 3.12 (s, 1H), 2.73-2.56 (m, 3H), 1.97-1.85 (m, 2H), 1.71-1.60 (m, 4H)

### General Procedure for Preparation of Intermediate 6 & Intermediate 6A:

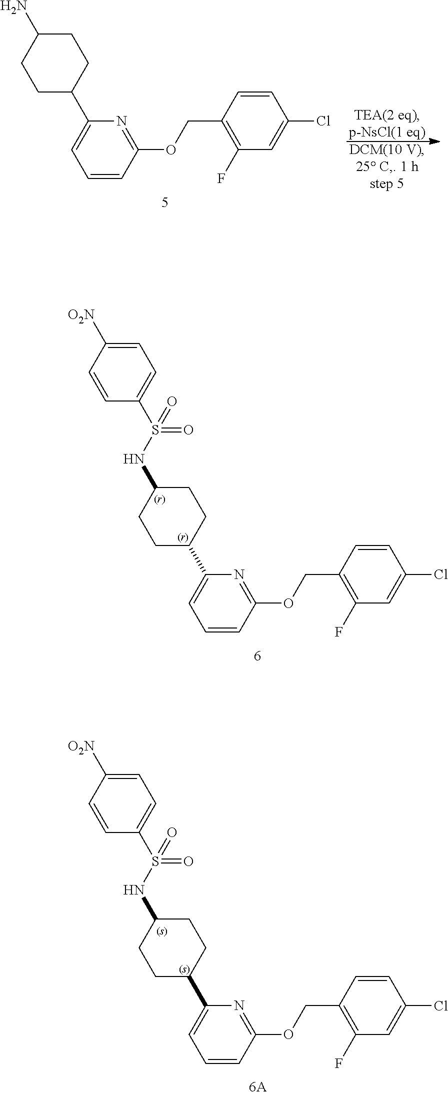

To a solution of 5 (2.1 g, 6.27 mmol, 1 eq) in DCM (42 mL) was added 4-nitrobenzenesulfonyl chloride (1.39 g, 6.27 mmol, 1 eq) and TEA (1.27 g, 12.54 mmol, 1.75 mL, 2 eq) at 25° C. The mixture was stirred at 25° C. for 1 hr. The reaction solution is filtered reaction mixture was diluted with H₂O 40 mL and extracted with DCM 35 mL, washed with brine 30 mL, dried over Na₂SO₄, filtered and concentrated under reduced pressure to give a residue. The residue was purified by column chromatography (SiO₂, Petroleum ether/Ethyl acetate=1/0 to 0/1, R_{f1}=0.51, R_{f2}=0.46). 6 (1.2 g, 2.31 mmol, 18.40% yield) was obtained as a white solid. 6A (2.8 g, 5.38 mmol, 42.93% yield) was obtained as a white solid.

LCMS: RT=2.38 min, MS cal.: 519.1, [M+H]⁺=520.0

¹H NMR (400 MHz, CHCl3-d) δ=8.46-8.35 (m, 2H), 8.13 (d, J=8.8 Hz, 2H), 7.50 (t, J=8.0 Hz, 1H), 7.43 (t, J=8.4 Hz, 1H), 7.17-7.09 (m, 2H), 6.69 (d, J=7.2 Hz, 1H), 6.62 (d, J=8.4 Hz, 1H), 5.39 (s, 2H), 4.53 (d, J=8.0 Hz, 1H), 3.39-3.26 (m, 1H), 2.59-2.44 (m, 1H), 2.05-1.87 (m, 4H), 1.57 (s, 2H), 1.35 (dd, J=3.2, 12.1 Hz, 2H)

### General Procedure for Preparation of Intermediate 7:

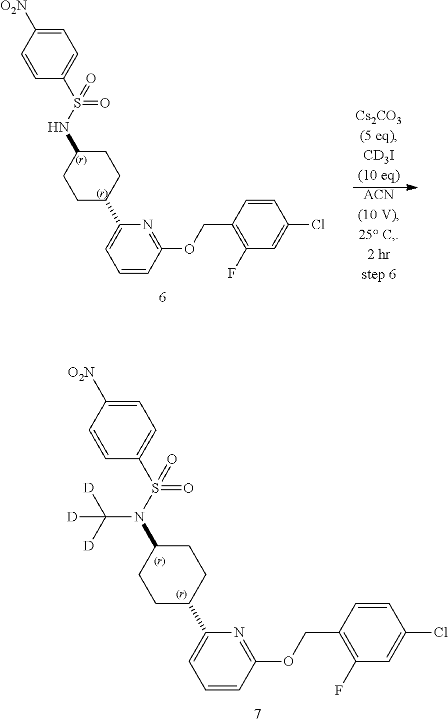

To a solution of 6 (550 mg, 1.06 mmol, 1 eq) in ACN (5.5 mL) and Cs₂CO₃ (1.72 g, 5.29 mmol, 5 eq) was added at 25° C. Trideuterio(iodo)methane (1.53 g, 10.58 mmol, 658.36 μL, 10 eq) was added to the reaction mixture. After the addition, the mixture was stirred at 25° C. for 2 hr. The mixture was stirred at 60° C. for 2 hr. TLC showed 6 was consumed completely. The mixture was diluted with H₂O (15 mL) extracted by EtOAc (5 mL×3). Then organic phase was washed by brine (10 mL). The organic were dried (Na₂SO₄), filtered. The organic phase was concentrated under reduced pressure at 45° C. to give 7 (200 mg, 665.09 μmol, 80.56% yield) as a yellow solid.

LCMS: RT=0.671 min, MS cal.: 536.1, [M+H]⁺=537.1.

¹H NMR (400 MHz, CHCl3-d) δ=8.36-8.27 (m, 2H), 8.00-7.92 (m, 2H), 7.44-7.39 (m, 1H), 7.36 (t, J=8.0 Hz, 1H), 7.06 (d, J=8.6 Hz, 2H), 6.60 (d, J=7.2 Hz, 1H), 6.53 (d, J=8.2 Hz, 1H), 5.32 (s, 2H), 3.86 (s, 1H), 2.39 (s, 1H), 1.87 (d, J=12.6 Hz, 2H), 1.65-1.49 (m, 6H)

### General Procedure for Preparation of Intermediate 8:

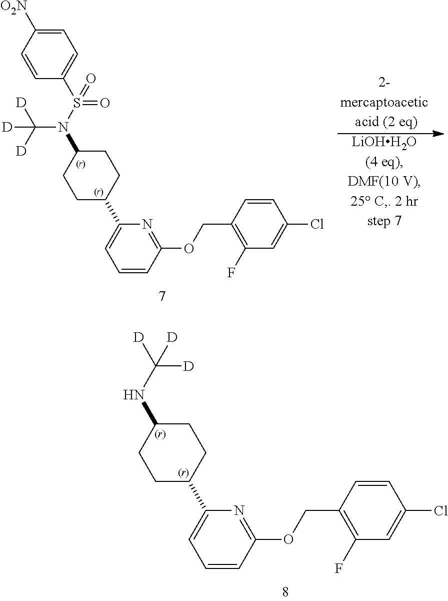

To a solution of 7 (500 mg, 931.07 μmol, 1 eq) in DMF (5 mL) was added LiOH·H₂O (156.27 mg, 3.72 mmol, 4 eq) and 2-sulfanylacetic acid (171.54 mg, 1.86 mmol, 127.07 μL, 2 eq) at 25° C. The mixture was stirred at 25° C. for 2 hr. TLC showed 7 was consumed completely. The reaction mixture was diluted with H₂O 10 mL and extracted with EtOAc 6 mL. The combined organic layers were washed with brine 10 mL, dried over Na₂SO₄, filtered, and concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 35%-80% B over 8.0 min). 8 (200 mg, 568.41 μmol, 61.05% yield) was obtained as a white solid.

LCMS: RT-0.436 min, MS cal.: 351.2, [M+H]⁺=352.1

¹H NMR (400 MHz, CHCl3-d) δ=7.62 (t, J=7.6 Hz, 1H), 7.57 (t, J=8.4 Hz, 1H), 7.47 (dd, J=2.0, 10.0 Hz, 1H), 7.33-7.28 (m, 1H), 6.85 (d, J=7.2 Hz, 1H), 6.66 (d, J=8.2 Hz, 1H), 5.37 (s, 2H), 2.59-2.52 (m, 1H), 2.30 (tt, J=4.0, 10.8 Hz, 1H), 2.01-1.93 (m, 2H), 1.84 (d, J=11.8 Hz, 2H), 1.51 (dq, J=2.7, 12.9 Hz, 2H), 1.18-1.01 (m, 2H)

### General Procedure for Preparation of Intermediate 9:

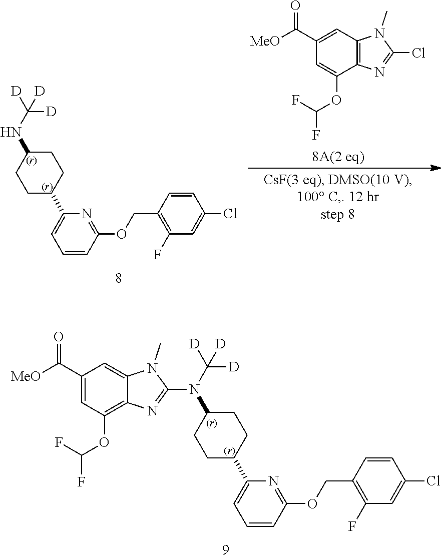

To a solution of 8 (100 mg, 284.20 μmol, 1 eq) in DMSO (1 mL) was added CsF (129.51 mg, 852.61 μmol, 3 eq) and 8A (165.21 mg, 568.41 μmol, 2 eq) at 25° C. The mixture was stirred at 100° C. for 12 hr. TLC showed 8 was consumed completely. The reaction mixture was filtered to give a residue. The reaction mixture was partitioned between EtOAc 8 mL and H₂O 10 mL. The organic phase was separated, washed with brine 10 mL, dried over Na₂SO₄, filtered, and concentrated under reduced pressure to give a residue. The residue was purified by column chromatography (SiO₂, Petroleum ether/Ethyl acetate=1/0 to 0/1). 9 (140 mg, crude) was obtained as a yellow solid.

### General Procedure for Preparation of Compound 372 (10):

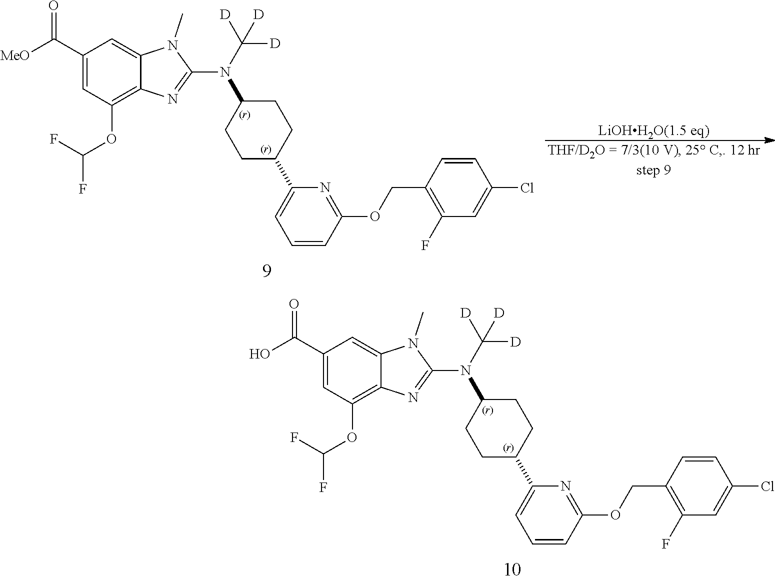

To a solution of 9 (130 mg, 171.60 μmol, 1 eq) in THF (0.7 mL) was added LiOH·H₂O (10.80 mg, 257.40 μmol, 1.5 eq) and H₂O (0.3 mL) at 25° C. The mixture was stirred at 25° C. for 12 hr. TLC showed 9 was consumed completely. The reaction mixture was filtered to give a residue. The residue was purified by prep-HPLC (column: Waters Xbridge BEH C18 100*25 mm*10 um; mobile phase: [H₂O (10 mM NH₄HCO₃)-ACN]; gradient: 40%-70% B over 8.0 min). 2-(((1r,4r)-4-(6-((4-Chloro-2-fluorobenzyl)oxy)pyridin-2-yl)cyclohexyl)(methyl-d3)amino)-4-(difluoromethoxy)-1-methyl-1H-benzo[d]imidazole-6-carboxylic acid (28.9 mg, 48.82 μmol, 28.45% yield) was obtained as a white solid.

LCMS: RT=2.984 min, MS cal.: 591.1, [M+H]⁺=592.2

HPLC: RT=14.294 min, purity: 97.02%

¹H NMR (400 MHz, MeOH-d4) δ=7.88 (s, 1H), 7.63 (s, 1H), 7.59 (t, J=7.6 Hz, 1H), 7.52 (t, J=8.0 Hz, 1H), 7.33 (t, J=37.6 Hz, 1H), 7.23-7.19 (m, 1H), 6.85 (d, J=7.2 Hz, 1H), 6.65 (d, J=8.2 Hz, 1H), 5.43 (s, 2H), 3.75 (s, 3H), 3.64 (br d, J=3.6 Hz, 1H), 2.65 (br d, J=1.6 Hz, 1H), 2.04 (br d, J=11.0 Hz, 4H), 1.91 (br d, J=10.0 Hz, 2H), 1.83-1.71 (m, 2H)

### Example A111—Preparation of Compound 373. 2-((4-(4-(4-Chloro-2-fluorobenzyl)-3,4-dihydro-2H-benzo[b][1,4]oxazin-6-yl)piperidin-1-yl)methyl)-4-(difluoromethoxy)-1-methyl-1H-benzo[d]imidazole-6-carboxylic acid (Compound 373) (5)

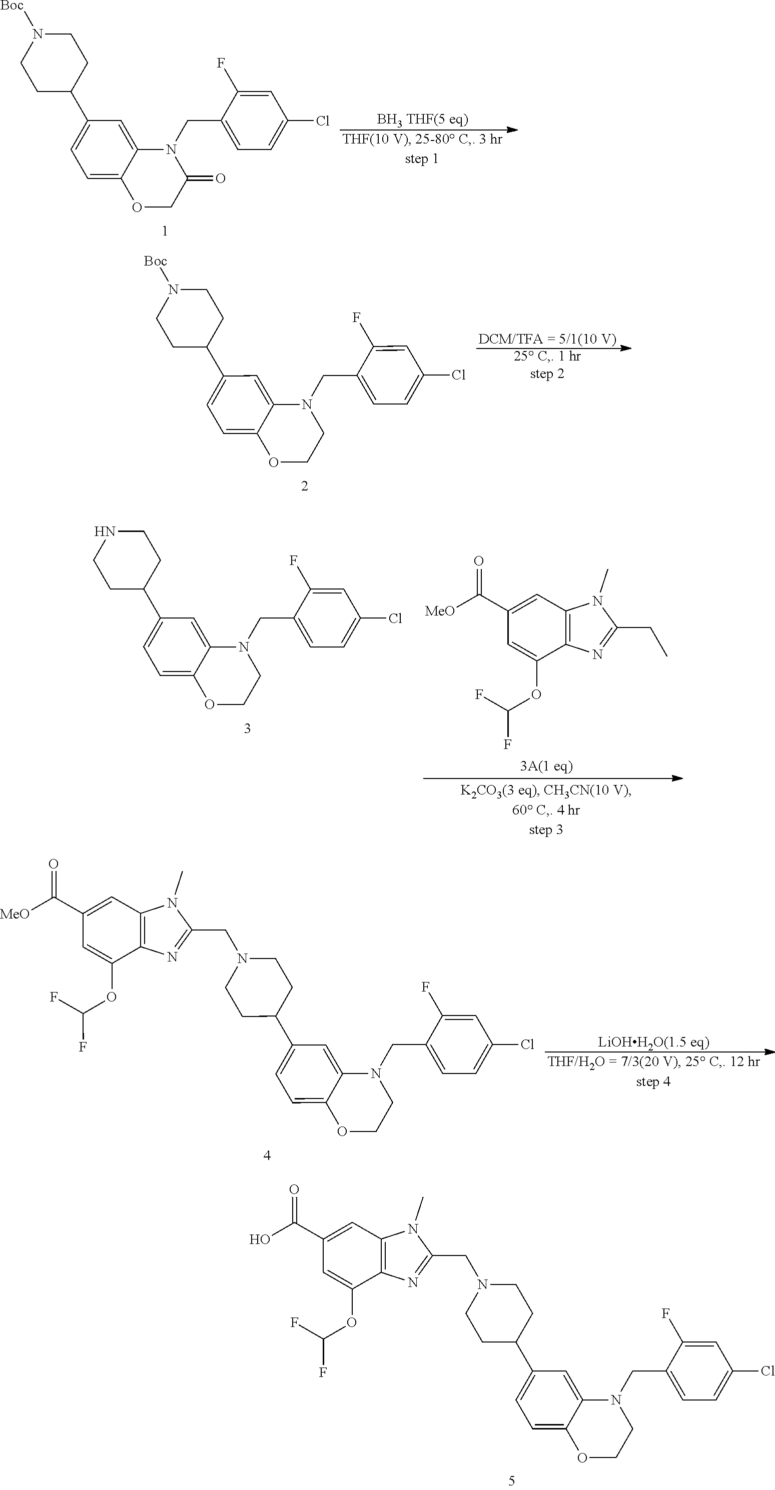 [SMILES: CC(C)(C)OC(=O)N1CCC(c2ccc3c(c2)N(Cc2ccc(Cl)cc2F)C(=O)CO3)CC1 ; CC(C)(C)OC(=O)N1CCC(c2ccc3c(c2)N(Cc2ccc(Cl)cc2F)CCO3)CC1 ; Fc1cc(Cl)ccc1CN1CCOc2ccc(C3CCNCC3)cc21 ; CCc1nc2c(OC(F)F)cc(C(=O)OC)cc2n1C ; COC(=O)c1cc(OC(F)F)c2nc(CN3CCC(c4ccc5c(c4)N(Cc4ccc(Cl)cc4F)CCO5)CC3)n(C)c2c1 ; Cn1c(CN2CCC(c3ccc4c(c3)N(Cc3ccc(Cl)cc3F)CCO4)CC2)nc2c(OC(F)F)cc(C(=O)O)cc21]

### General Procedure for Preparation of Intermediate 2:

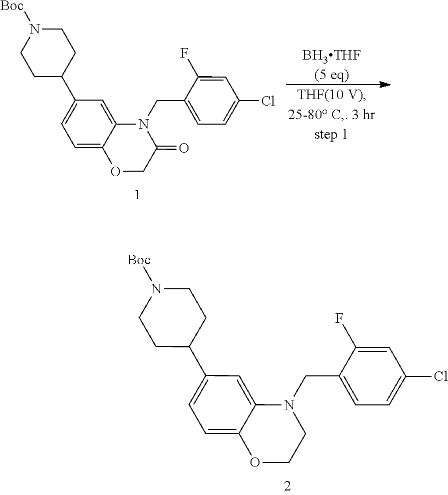 [SMILES: CC(C)(C)OC(=O)N1CCC(c2ccc3c(c2)N(Cc2ccc(Cl)cc2F)C(=O)CO3)CC1 ; CC(C)(C)OC(=O)N1CCC(c2ccc3c(c2)N(Cc2ccc(Cl)cc2F)CCO3)CC1]

To a mixture of 1 (700 mg, 1.47 mmol, 1 eq) in THF (7 mL) was added BH₃·THF (1 M, 7.37 mL, 5 eq). After the addition, the mixture was stirred at 25° C. for 1 hr. Then the mixture was stirred at 80° C. for 2 hr. LCMS (Product, RT=1.741 min) showed 95% of product 1 was detected. The reaction was cooled to 25° C. and quenched in an ice bath with the addition of MeOH (anhydrous 20 mL). The reaction was concentrated and then dissolved with EtOAc 10 ml. The reaction was washed with aq. Na₂CO₃ 30 mL, extracted with EtOAc (20 mL*3). The combined organic layers were washed with brine 20 mL, dried over Na₂SO₄, filtered and concentrated under reduced pressure to give a residue. 2 (600 mg, 1.24 mmol, 83.90% yield, 95% purity) was obtained as a colorless oil.

LCMS: RT=1.741 min, MS cal.: 460.2/462.2, [M−55]⁺=405/406.9

LCMS: RT=1.746 min, MS cal.: 460.2/462.2, [M−55]⁺=404.9/406.9

1H NMR (400 MHz, CHCl3-d) δ=7.26-7.19 (m, 1H), 7.15-7.08 (m, 2H), 6.77 (d, J=8.0 Hz, 1H), 6.55-6.44 (m, 2H), 4.45 (s, 2H), 4.29-4.23 (m, 2H), 4.23-4.01 (m, 2H), 3.40-3.34 (m, 2H), 2.74 (br t, J=12.4 Hz, 2H), 2.47 (br t, J=12.0 Hz, 1H), 1.74 (br d, J=12.8 Hz, 2H), 1.62-1.52 (m, 2H), 1.48 (s, 8H)

### General Procedure for Preparation of Intermediate 3:

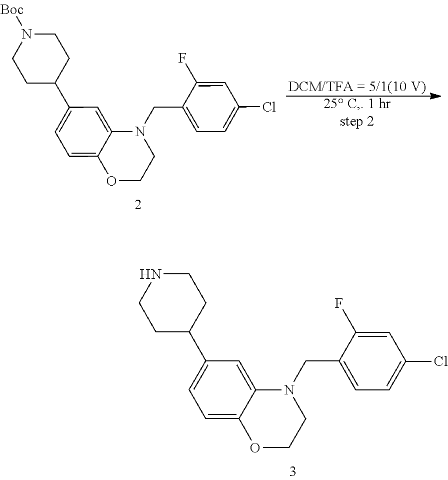

To a solution of 2 (550 mg, 1.19 mmol, 1 eq) in DCM (5.5 mL) was added TFA (1.1 mL). The mixture was stirred at 25° C. for 1 hr. TLC indicated 2 was consumed completely, and one major new spot with larger polarity was detected. The reaction mixture was concentrated under reduced pressure to give a residue. 3 (800 mg, crude, TFA) was obtained as a yellow oil.

LCMS: RT=1.127 min, MS cal.: 360.1/362.1, [M+1]⁺=361/362.9

### General Procedure for Preparation of Intermediate 4:

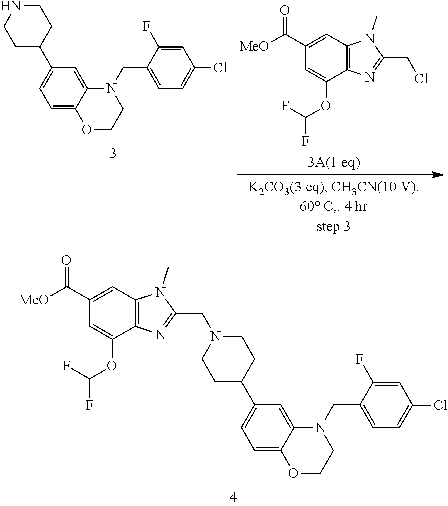 [SMILES: Fc1cc(Cl)ccc1CN1CCOc2ccc(C3CCNCC3)cc21 ; COC(=O)c1cc(OC(F)F)c2nc(CCl)n(C)c2c1 ; COC(=O)c1cc(OC(F)F)c2nc(CN3CCC(c4ccc5c(c4)N(Cc4ccc(Cl)cc4F)CCO5)CC3)n(C)c2c1]

A mixture of 3 (300 mg, 631.74 μmol, 1 eq, TFA), 3A (192.48 mg, 631.74 μmol, 1 eq), K₂CO₃ (261.93 mg, 1.90 mmol, 3 eq) in CH₃CN (3 mL) was degassed and purged with N₂ for 3 times, and then the mixture was stirred at 60° C. for 4 hr under N₂ atmosphere. LCMS (Product, RT=1.709 min) showed 76% of product 1 was detected. The mixture was diluted with H₂O (10 ml) and then extracted with EtOAc (20 mL*3). The combined organic layers were washed with brine 20 mL, dried over Na₂SO₄, filtered and concentrated under reduced pressure to give a residue. The residue was purified by column chromatography (SiO₂, Petroleum ether/Ethyl acetate=10/1 to 0/1). 4 (120 mg, 137.35 μmol, 21.74% yield, 72% purity) was obtained as a green solid.

LCMS: RT=1.710 min, MS cal.: 628.2/629.2, [M+H]⁺=629.1/631.0

1H NMR (400 MHz, CHCl3-d) δ=7.98 (d, J=1.2 Hz, 1H), 7.71 (s, 1H), 7.31 (t, J=74.4 Hz, 1H), 7.24 (t, J=8.0 Hz, 1H), 7.15-7.06 (m, 2H), 6.76 (d, J=8.2 Hz, 1H), 6.52 (dd, J=1.6 Hz, 8.3 Hz, 1H), 6.46 (d, J=1.6 Hz, 1H), 4.45 (s, 2H), 4.26-4.23 (m, 2H), 3.98 (s, 3H), 3.97 (s, 3H), 3.86 (br s, 2H), 3.39-3.35 (m, 2H), 2.97-2.88 (m, 3H), 2.44-2.34 (m, 1H), 2.30-2.20 (m, 2H), 1.78 (br s, 1H), 1.75 (br s, 1H)

### General Procedure for Preparation of Compound 373 (5):

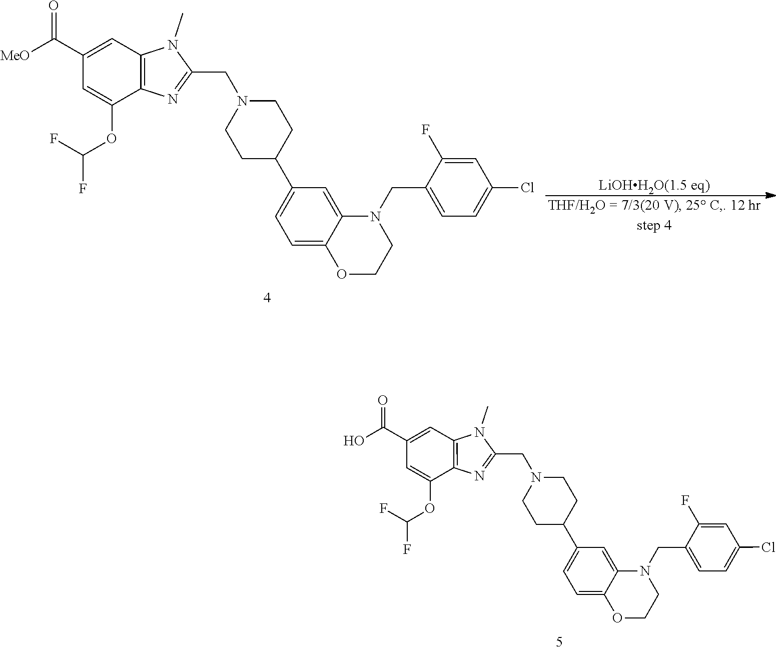 [SMILES: COC(=O)c1cc(OC(F)F)c2nc(CN3CCC(c4ccc5c(c4)N(Cc4ccc(Cl)cc4F)CCO5)CC3)n(C)c2c1 ; Cn1c(CN2CCC(c3ccc4c(c3)N(Cc3ccc(Cl)cc3F)CCO4)CC2)nc2c(OC(F)F)cc(C(=O)O)cc21]

A mixture of 4 (120 mg, 190.76 μmol, 1 eq), LiOH·H₂O (12.01 mg, 286.14 μmol, 1.5 eq) in

THF (1.68 mL) and H₂O (0.72 mL) was degassed and purged with N₂ for 3 times, and then the mixture was stirred at 25° C. for 12 hr under N₂ atmosphere. LCMS (Product, RT=1.216 min) showed 62% of product 1 was detected. The reaction mixture was purified directly. The residue was purified by prep-HPLC (neutral condition column: Waters Xbridge Prep OBD C18 150*40 mm*10 um; mobile phase: [H₂O (10 mM NH₄HCO₃)-ACN]; gradient: 25%-75% B over 8.0 min). 2-((4-(4-(4-Chloro-2-fluorobenzyl)-3,4-dihydro-2H-benzo[b][1,4]oxazin-6-yl)piperidin-1-yl)methyl)-4-(difluoromethoxy)-1-methyl-1H-benzo[d]imidazole-6-carboxylic acid (20.63 mg, 33.46 μmol, 17.54% yield, 99.76% purity) was obtained as a white solid.

LCMS: RT=1.216 min, MS cal.: 614.2/615.2, [M+H]⁺=615.1/617.0

HPLC: RT=12.326 min, purity: 99.76%

1H NMR (400 MHz, MeOH-d4) δ=8.09 (d, J=1.2 Hz, 1H), 7.68 (s, 1H), 7.31 (t, J=8.0 Hz, 1H), 7.24 (t, J=74.4 Hz, 1H), 7.23 (m, 1H), 7.14 (dd, J=1.6, 8.3 Hz, 1H), 6.63 (d, J=8.0 Hz, 1H), 6.50-6.44 (m, 2H), 4.48 (s, 2H), 4.22-4.19 (m, 2H), 3.99 (s, 3H), 3.96 (s, 2H), 3.40-3.37 (m, 2H), 3.05 (br d, J=11.6 Hz, 2H), 2.42-2.32 (m, 3H), 1.76-1.61 (m, 4H)

### Example A112—Preparation of Compound 374. 2-((4-(6-((4-Chloro-2-fluorobenzyl)oxy)pyrazin-2-yl)piperidin-1-yl)methyl)-4-(difluoromethoxy)-1-methyl-1H-benzo[d]imidazole-6-carboxylic acid (Compound 374) (7)

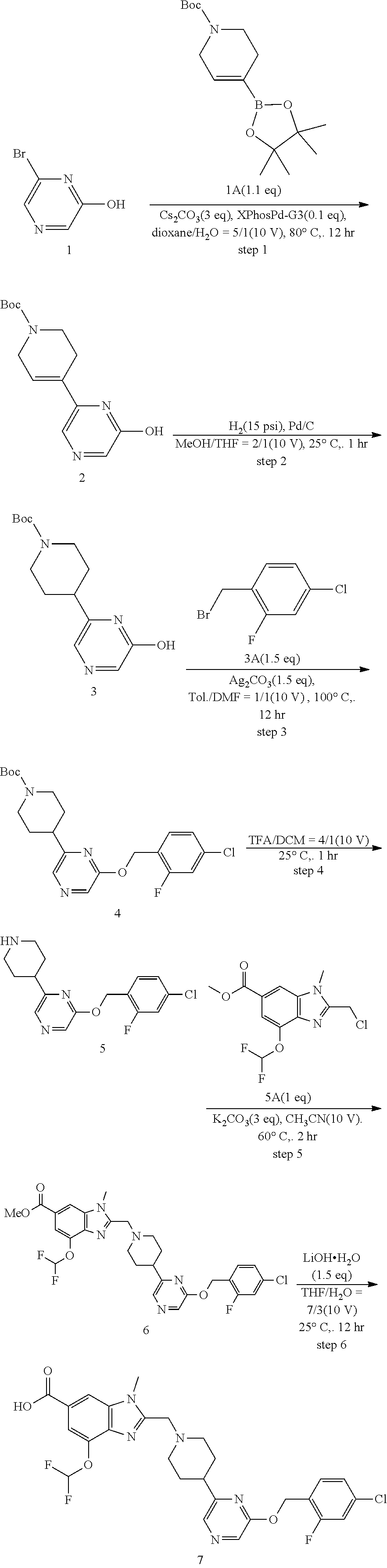

### General Procedure for Preparation of Intermediate 2:

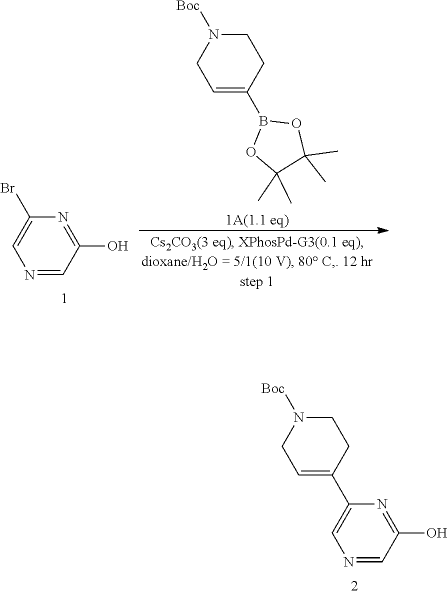 [SMILES: Oc1cncc(Br)n1 ; CC(C)(C)OC(=O)N1CC=C(B2OC(C)(C)C(C)(C)O2)CC1 ; CC(C)(C)OC(=O)N1CC=C(c2cncc(O)n2)CC1]

To a mixture of 1 (1 g, 5.71 mmol, 1 eq), 1A (1.94 g, 6.29 mmol, 1.1 eq) and Cs₂CO₃ (5.59 g, 17.14 mmol, 3 eq) in dioxane (10 mL) and H₂O (2 mL) was added Xphos-Pd-G3 (483.73 mg, 571.48 μmol, 0.1 eq) at 25° C. The mixture was de-gassed under reduced pressure and recharged with N₂. The mixture was stirred at 80° C. for 12 hours. LC-MS (ET83210-129-P1A) showed 1 was consumed completely and one main peak with desired mass was detected. The residue was poured into water (50 mL). The aqueous phase was extracted with ethyl acetate (20 mL*3). The combined organic phase was washed with saturated brine (50 mL*1), dried with anhydrous Na₂SO₄, filtered and concentrated in vacuum. The residue was purified by column chromatography (SiO₂, PE/EtOAc=1/0 to 0/1). 2 (880 mg, 3.09 mmol, 27.02% yield) was obtained as a yellow solid.

LCMS: RT=0.359 min, MS cal.: 277.1, [M+H]⁺=278.1

HPLC: product: RT=2.145 min, purity: 97.33%

¹H NMR (400 MHz, MeOH-d4) δ=7.94 (s, 1H), 7.70 (br s, 1H), 6.53 (br s, 1H), 4.12 (br s, 2H), 3.63 (br t, J=4.8 Hz, 2H), 2.52 (br s, 2H), 1.49 (s, 9H)

### General Procedure for Preparation of Intermediate 3:

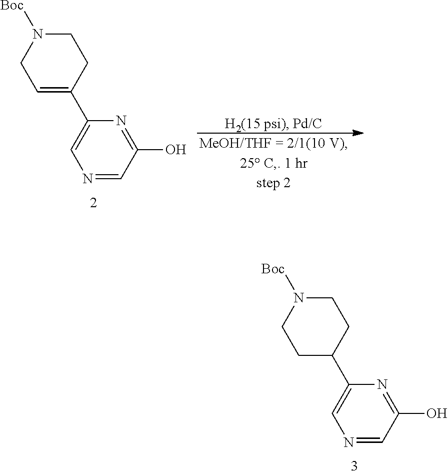 [SMILES: CC(C)(C)OC(=O)N1CC=C(c2cncc(O)n2)CC1 ; CC(C)(C)OC(=O)N1CCC(c2cncc(O)n2)CC1]

To a solution 2 in MeOH (1 mL) was added Pd/C (100 mg, 10% purity) under N₂ atmosphere. The suspension was degassed and purged with H₂ for 3 times. LC-MS (ET83210-131-P1E) showed 2 was consumed completely and one main peak with desired mass was detected. The mixture was filtered and concentrated in vacuum. The residue was purified by column chromatography (SiO₂, Petroleum ether:Ethyl acetate=1/0 to 0/1). 3 (130 mg, 451.34 μmol, 31.29% yield) was obtained as a yellow solid.

LCMS (3): RT=0.353 min, MS cal.: 279.2, [M+H−t-Bu]⁺=224.2

HPLC: product: RT=1.382 min, purity: 96.98%

¹H NMR (400 MHz, MeOH-d4) δ=7.89 (s, 1H), 7.40 (s, 1H), 4.22 (br d, J=13.6 Hz, 2H), 2.85 (br s, 2H), 2.70 (tt, J=3.6, 12.0 Hz, 1H), 1.88 (br d, J=12.8 Hz, 2H), 1.70-1.59 (m, 2H), 1.47 (s, 9H)

### General Procedure for Preparation of Intermediate 4:

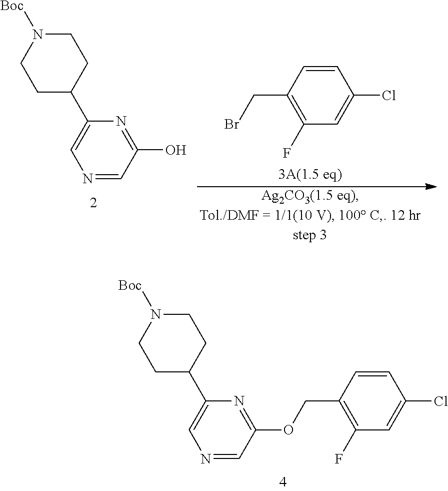 [SMILES: CC(C)(C)OC(=O)N1CCC(c2cncc(O)n2)CC1 ; Fc1cc(Cl)ccc1CBr ; CC(C)(C)OC(=O)N1CCC(c2cncc(OCc3ccc(Cl)cc3F)n2)CC1]

To a solution of 3 (120 mg, 429.59 μmol, 1 eq) in DMF (1 mL) and Toluene (1 mL) was added 3A (144.00 mg, 644.39 μmol, 1.5 eq) and Ag₂CO₃ (177.69 mg, 644.39 μmol, 29.24 μL, 1.5 eq). The mixture was stirred at 100° C. for 12 hr. LC-MS showed 3 was consumed completely and one main peak with desired mass was detected. The residue was poured into water (50 mL). The aqueous phase was extracted with ethyl acetate (10 mL*3). The combined organic phase was washed with saturated brine (50 mL*1), dried with anhydrous Na₂SO₄, filtered, and concentrated in vacuum. The residue was purified by column chromatography (SiO₂, Petroleum ether:Ethyl acetate=1/0 to 1/1). 4 (80 mg, 157.77 μmol, 36.72% yield) was obtained as a yellow solid.

LCMS: RT=0.897 min, MS cal.: 421.2, [M+H−t-Bu]⁺=366.2

HPLC: product: RT=3.258 min, purity: 83.20%

¹H NMR (400 MHz, MeOH-d4) δ=8.06 (d, J=5.6 Hz, 2H), 7.51 (t, J=8.0 Hz, 1H), 7.26-7.17 (m, 2H), 5.45 (s, 2H), 4.19 (br d, J=13.2 Hz, 2H), 2.91 (tdd, J=3.6, 7.6, 15.2 Hz, 3H), 1.86-1.78 (m, 2H), 1.76-1.64 (m, 2H), 1.49 (s, 9H)

### General Procedure for Preparation of Intermediate 5:

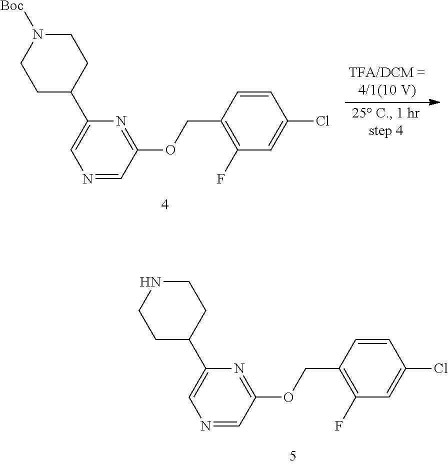 [SMILES: CC(C)(C)OC(=O)N1CCC(c2cncc(OCc3ccc(Cl)cc3F)n2)CC1 ; Fc1cc(Cl)ccc1COc1cncc(C2CCNCC2)n1]

To a solution of 4 (69 mg, 163.55 μmol, 1 eq) in DCM (0.8 mL) was added TFA (307.00 mg, 2.69 mmol, 0.2 mL, 16.46 eq). The mixture was stirred at 25° C. for 1 hr. LC-MS (ET83210-136-PIA) showed 4 was consumed completely and one main peak with desired mass was detected. The residue was poured into NaHCO₃until PH>7. The aqueous phase was extracted with dichloromethane (10 mL*3). The combined organic phase was washed with saturated brine (50 mL*1), dried with anhydrous Na₂SO₄, filtered, and concentrated in vacuum. The crude product was used into the next step without further purification. 5 (62 mg, crude) was obtained as a yellow solid.

LCMS: RT=0.373 min, MS cal.: 321.1 [M+H]⁺=322.1

HPLC: product: RT=2.141 min, purity: 95.86%

¹H NMR (400 MHz, MeOH-d4) δ=8.10 (d, J=8.8 Hz, 2H), 7.55 (t, J=8.0 Hz, 1H), 7.29-7.22 (m, 2H), 5.48 (s, 2H), 3.34 (br d, J=2.8 Hz, 2H), 3.03-2.89 (m, 3H), 2.04-1.85 (m, 4H)

### General Procedure for Preparation of Intermediate 6:

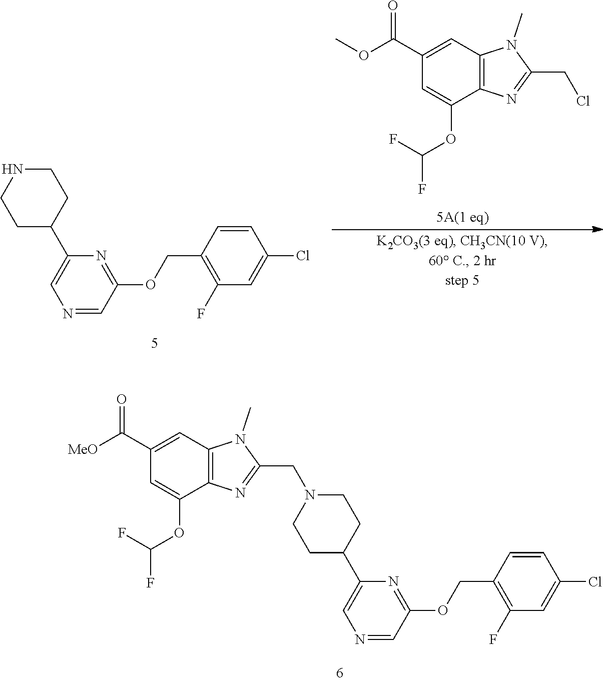 [SMILES: Fc1cc(Cl)ccc1COc1cncc(C2CCNCC2)n1 ; COC(=O)c1cc(OC(F)F)c2nc(CCl)n(C)c2c1 ; COC(=O)c1cc(OC(F)F)c2nc(CN3CCC(c4cncc(OCc5ccc(Cl)cc5F)n4)CC3)n(C)c2c1]

To a solution of 5A (47.34 mg, 155.39 μmol, 1 eq) in ACN (1 mL) was added K₂CO₃ (64.43 mg, 466.16 μmol, 3 eq) and 5 (50 mg, 155.39 μmol, 1 eq). The mixture was de-gassed under reduced pressure and recharged with N₂. The mixture was stirred at 60° C. for 2 hr. LC-MS showed 5 was consumed completely and one main peak with desired mass was detected. The mixture was filtered and concentrated in vacuum. The residue was purified by prep-TLC (SiO₂, Petroleum ether:Ethyl acetate=2:1, R_{f}=0.40). 6 (65 mg, 110.17 μmol, 70.90% yield) was obtained as a yellow solid.

LCMS: RT=0.601 min, MS cal.: 589.2 [M+H]⁺=590.1

¹H NMR (400 MHz, DMSO-d6) δ=8.17 (d, J=3.2 Hz, 2H), 8.13 (d, J=1.2 Hz, 1H), 7.66 (t, J=74.4, 2.0 Hz, 1H), 7.54 (s, 1H), 7.51-7.45 (m, 1H), 7.31 (dd, J=2.0, 8.4 Hz, 1H), 5.41 (s, 2H), 3.99 (s, 3H), 3.91 (s, 3H), 3.88 (s, 2H), 2.97 (br d, J=11.2 Hz, 2H), 2.76-2.65 (m, 1H), 2.30-2.20 (m, 2H), 1.85-1.69 (m, 4H)

### General Procedure for Preparation of Compound 374 (7):

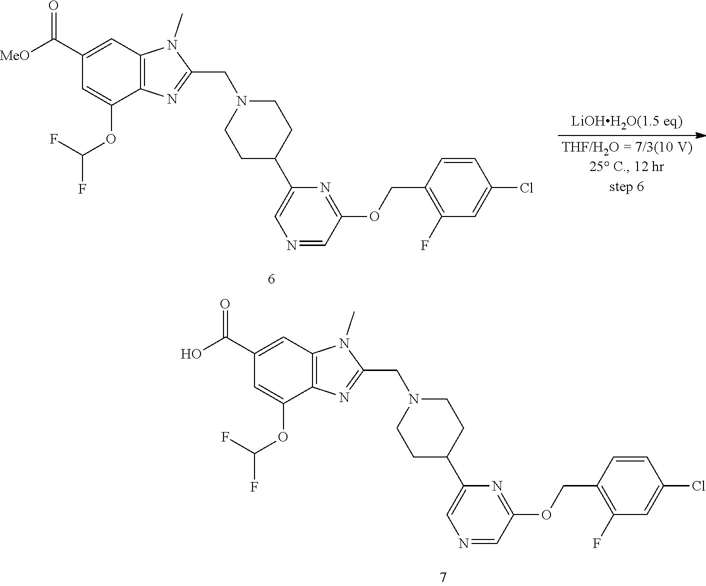 [SMILES: COC(=O)c1cc(OC(F)F)c2nc(CN3CCC(c4cncc(OCc5ccc(Cl)cc5F)n4)CC3)n(C)c2c1 ; Cn1c(CN2CCC(c3cncc(OCc4ccc(Cl)cc4F)n3)CC2)nc2c(OC(F)F)cc(C(=O)O)cc21]

To a solution of 6 (50 mg, 84.75 μmol, 1 eq) in THF (0.7 mL) and H₂O (0.3 mL) was added LiOH·H₂O (5.33 mg, 127.12 μmol, 1.5 eq). The mixture was stirred at 25° C. for 15 hr. LC-MS showed 6 was consumed completely and one main peak with desired mass was detected. The mixture was filtered and concentrated in vacuum. The residue was purified by prep-HPLC (column: Waters Xbridge Prep OBD C₁₈ 150*40 mm*10 um; mobile phase: [H₂O (10 mM NH₄HCO₃)-ACN]; gradient: 20%-60% B over 8.0 min). 2-((4-(6-((4-Chloro-2-fluorobenzyl)oxy)pyrazin-2-yl)piperidin-1-yl)methyl)-4-(difluoromethoxy)-1-methyl-1H-benzo[d]imidazole-6-carboxylic acid (29 mg, 50.05 μmol, 59.06% yield) was obtained as a yellow solid.

LCMS: RT=2.617 min, MS cal.: 575.2, [M+H]⁺=576.2

HPLC: product: RT=11.016 min, purity: 99.41%

¹H NMR (400 MHz, DMSO-d6) δ=8.17 (d, J=2.4 Hz, 2H), 8.07 (s, 1H), 7.63 (t, J=74.4, 2.0 Hz, 1H), 7.52 (s, 1H), 7.30 (dd, J=2.0, 8.4 Hz, 1H), 5.40 (s, 2H), 3.97 (s, 3H), 3.86 (s, 2H), 2.96 (br d, J=11.2 Hz, 2H), 2.74-2.65 (m, 1H), 2.30-2.18 (m, 2H), 1.88-1.66 (m, 4H)

### Example A113—Preparation of Compound 375. 2-((4-(2-((4-Chloro-2-fluorobenzyl)oxy)-5-fluoropyrimidin-4-yl)piperidin-1-yl)methyl)-4-(difluoromethoxy)-1-methyl-1H-benzo[d]imidazole-6-carboxylic acid (Compound 375) (6)

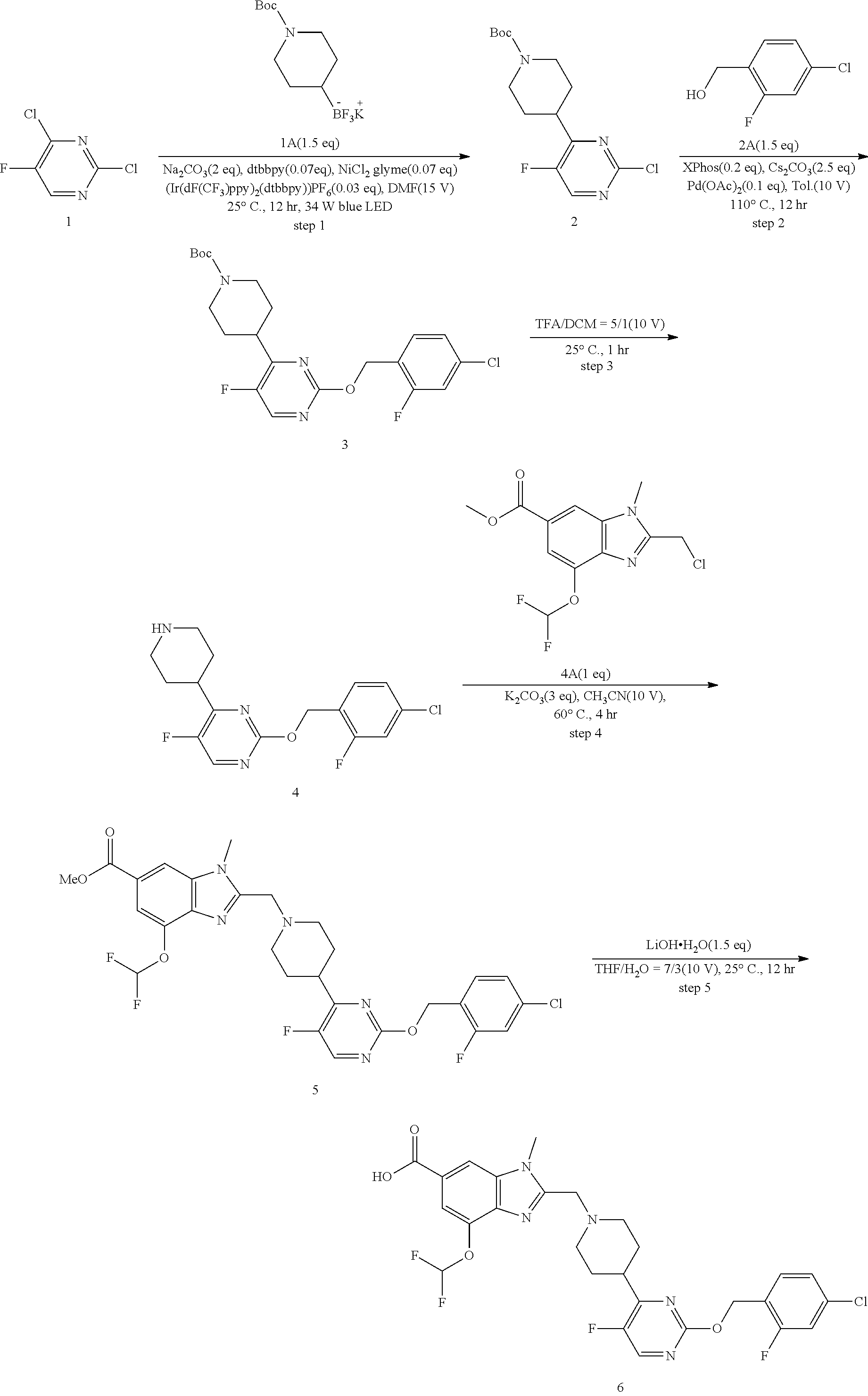

### General Procedure for Preparation of Intermediate 2:

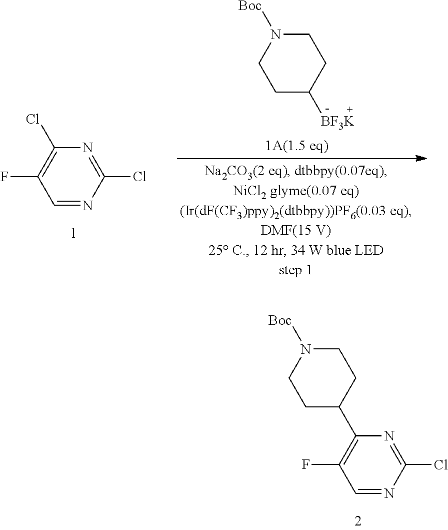

To a mixture of 1 (200 mg, 1.20 mmol, 1 eq) and 1A (523.14 mg, 1.80 mmol, 1.5 eq, K) in DMA (4 mL) was added Na₂CO₃ (253.91 mg, 2.40 mmol, 2 eq), dtbbpy (22.50 mg, 83.85 μmol, 0.07 eq), dichloronickel; 1,2-dimethoxyethane (18.42 mg, 83.85 μmol, 0.07 eq) and bis[3,5-difluoro-2-[5-(trifluoromethyl)-2-pyridyl]phenyl]iridium(1+); 4-tert-butyl-2-(4-tert-butyl-2-pyridyl)pyridine; hexafluorophosphate (40.32 mg, 35.93 μmol, 0.03 eq) 25° C. under Ar. The mixture was stirred at 25° C. at 34W blue LED for 12 hours. The color changes to brown. TLC (SiO₂, PE:EA=5:1, R_{f}=0.50) showed the reaction was completed. The reaction mixture was quenched by addition water 10 mL, and then extracted with EA 30 mL (10 mL*3). The combined organic layers were washed with brine 30 mL, dried over Na₂SO₄, filtered and concentrated under reduced pressure to give a residue. The residue was purified by prep-TLC (SiO₂, PE:EA=5:1, R_{f}=0.45). 2 (440 mg, 1.23 mmol, 51.40% yield) was obtained as a white solid.

HPLC: RT=2.501 min, purity: 88.37%

¹H NMR (400 MHz, MeOH-d4) δ=8.54 (d, J=1.6 Hz, 1H), 4.20 (d, J=13.6 Hz, 2H), 3.30-3.23 (m, 1H), 3.03-2.82 (m, 2H), 1.87-1.68 (m, 4H), 1.48 (s, 9H)

### General Procedure for Preparation of Intermediate 3:

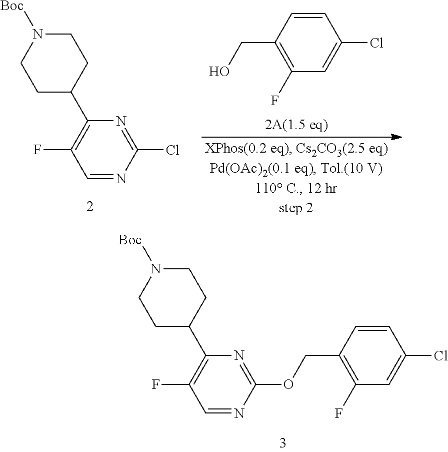 [SMILES: CC(C)(C)OC(=O)N1CCC(c2nc(Cl)ncc2F)CC1 ; OCc1ccc(Cl)cc1F ; CC(C)(C)OC(=O)N1CCC(c2nc(OCc3ccc(Cl)cc3F)ncc2F)CC1]

To a mixture of 2 (220 mg, 696.71 μmol, 1 eq), Cs₂CO₃ (567.50 mg, 1.74 mmol, 2.5 eq) and 2A (223.75 mg, 1.39 mmol, 2 eq) in Toluene (3 mL) was added Pd(OAc)₂ (15.64 mg, 69.67 μmol, 0.1 eq) and Xphos (66.43 mg, 139.34 μmol, 0.2 eq) under N₂. The mixture was stirred at 110° C. for 12 hours. The color changes to brown. LCMS showed the reaction was completed. The reaction mixture was quenched by addition water 30 mL, and then extracted with EA 45 mL (15 mL*3). The combined organic layers were washed with brine 30 mL, dried over Na₂SO₄, filtered, and concentrated under reduced pressure to give a residue. The residue was purified by prep-HPLC (column: Waters Xbridge BEH C18 100*30 mm*10 um; mobile phase: [H₂O (10 mM NH₄HCO₃)-ACN]; gradient: 45%-80% B over 8.0 min). 3 (190 mg, 429.64 μmol, 30.83% yield) was obtained as a white solid.

LCMS: RT=0.656 min, MS cal.: 439.1, [M+H]⁺=384.2

HPLC: RT=3.267 min, purity: 99.47%

¹H NMR (400 MHz, MeOH-d4) δ=8.39 (d, J=2.0 Hz, 1H), 7.55-7.50 (m, 1H), 7.26-7.20 (m, 2H), 5.44 (s, 2H), 4.19 (d, J=13.2 Hz, 2H), 3.29-3.18 (m, 1H), 2.92 (s, 2H), 1.82-1.69 (m, 4H), 1.49 (s, 9H)

### General Procedure for Preparation of Intermediate 4:

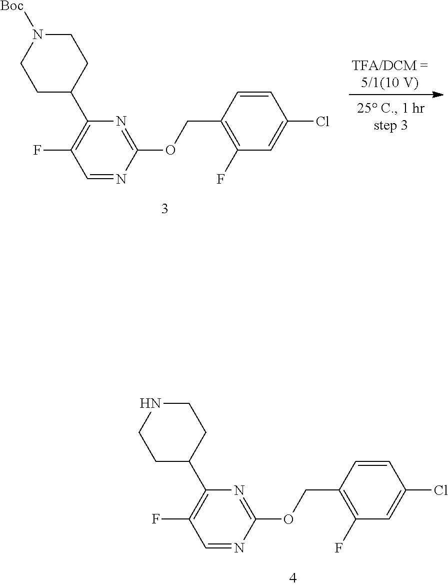 [SMILES: CC(C)(C)OC(=O)N1CCC(c2nc(OCc3ccc(Cl)cc3F)ncc2F)CC1 ; Fc1cc(Cl)ccc1COc1ncc(F)c(C2CCNCC2)n1]

To a mixture of 3 (190 mg, 431.93 μmol, 1 eq) in DCM (5 mL) and TFA (1 mL) was stirred at 25° C. for 1 hour. The color changes to yellow. LCMS showed the reaction was completed. The reaction mixture was quenched by addition Sat. NaHCO₃ 10 mL at 25° C., and then diluted with EA 5 mL and extracted with EA 30 mL (10 mL*3). The combined organic layers were washed with brine 30 mL, dried over Na₂SO₄, filtered, and concentrated under reduced pressure to give a residue. 4 (135 mg, 393.36 μmol, 91.07% yield) was obtained as yellow oil.

LCMS: RT=0.380 min, MS cal.: 339.1, [M+H]⁺=340.1

HPLC: RT=1.840 min, purity: 99.01%

¹H NMR (400 MHz, MeOH-d4) δ=8.41 (d, J=2.0 Hz, 1H), 7.55 (t, J=8.4 Hz, 1H), 7.28-7.21 (m, 2H), 5.46 (s, 2H), 3.30-3.21 (m, 3H), 1.99-1.85 (m, 4H)

### General Procedure for Preparation of Intermediate 5:

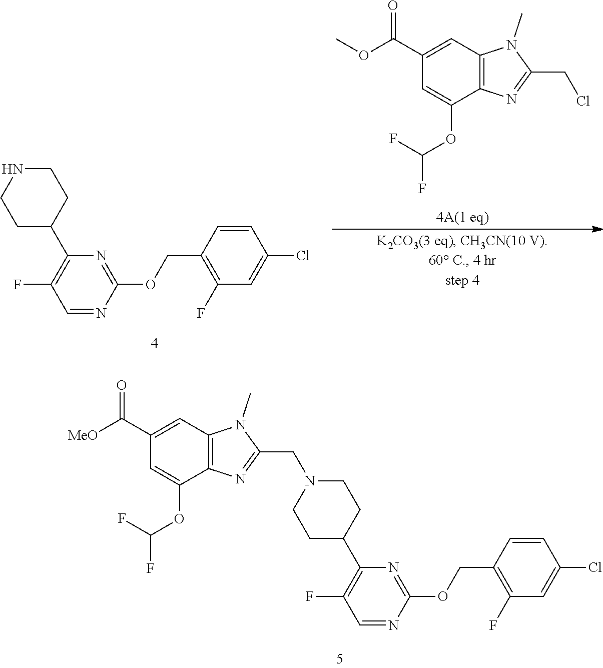 [SMILES: Fc1cc(Cl)ccc1COc1ncc(F)c(C2CCNCC2)n1 ; COC(=O)c1cc(OC(F)F)c2nc(CCl)n(C)c2c1 ; COC(=O)c1cc(OC(F)F)c2nc(CN3CCC(c4nc(OCc5ccc(Cl)cc5F)ncc4F)CC3)n(C)c2c1]

A mixture of 4 (135 mg, 397.33 μmol, 1.2 eq), 4A (100.88 mg, 331.11 μmol, 1 eq) and K₂CO₃ (137.28 mg, 993.33 μmol, 3 eq) in ACN (4 mL) was stirred at 60° C. for 1 hour. The color changes to brown. TLC (SiO₂, PE:EA=1:1, R_{f}=0.50) showed the reaction was completed. The mixture was filtered and concentrated in vacuum. The residue was purified by prep-TLC (SiO₂, PE:EA=1:1, R_{f}=0.45). 5 (180 mg, 268.32 μmol, 81.04% yield) was obtained as a white solid.

HPLC: RT=3.211 min, purity: 90.63%

¹H NMR (400 MHz, CHCl3-d) δ=8.25 (s, 1H), 7.99 (s, 1H), 7.72 (s, 1H), 7.48-7.45 (m, 1H), 7.30 (t, J=74.4 Hz, 1H), 7.16-7.09 (m, 2H), 5.41 (s, 2H), 4.01-3.96 (m, 6H), 3.90 (s, 2H), 2.99 (d, J=1.6 Hz, 3H), 2.41-2.24 (m, 2H), 2.03-1.88 (m, 2H), 1.86-1.77 (m, 2H)

### General Procedure for Preparation of Compound 375 (6):

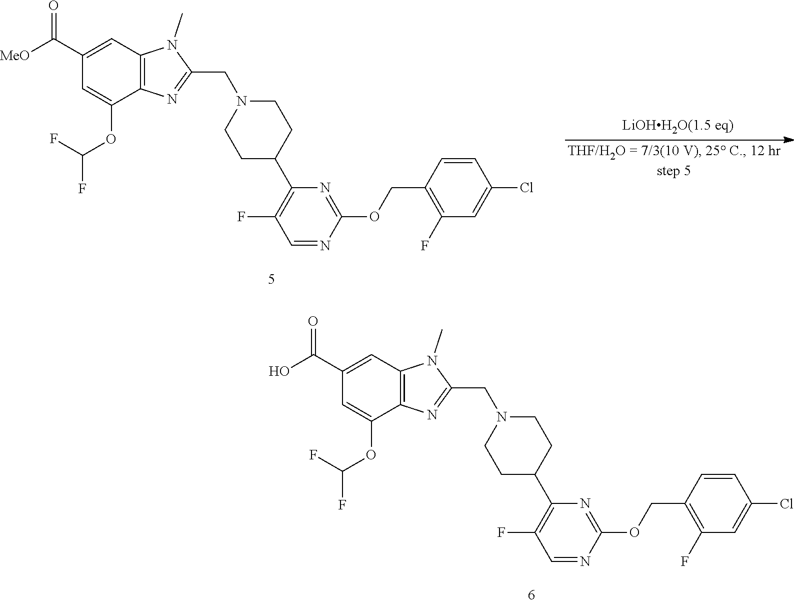 [SMILES: COC(=O)c1cc(OC(F)F)c2nc(CN3CCC(c4nc(OCc5ccc(Cl)cc5F)ncc4F)CC3)n(C)c2c1 ; Cn1c(CN2CCC(c3nc(OCc4ccc(Cl)cc4F)ncc3F)CC2)nc2c(OC(F)F)cc(C(=O)O)cc21]

To a mixture of 5 (140 mg, 230.27 μmol, 1 eq) and LiOH·H₂O (14.49 mg, 345.40 μmol, 1.5 eq) in THF (3.5 mL) and H₂O (1.5 mL) was stirred at 25° C. for 12 hours. The color changes to yellow. LCMS showed 95% of desired product was detected. The mixture was concentrated in vacuum. The residue was purified by prep-HPLC (column: Waters Xbridge BEH C₁₈ 100*30 mm*10 um; mobile phase: [H₂O (10 mM NH₄HCO₃)-ACN]; gradient: 30%-50% B over 8.0 min). 2-((4-(2-((4-Chloro-2-fluorobenzyl)oxy)-5-fluoropyrimidin-4-yl)piperidin-1-yl)methyl)-4-(difluoromethoxy)-1-methyl-1H-benzo[d]imidazole-6-carboxylic acid (29.64 mg, 49.68 μmol, 21.58% yield) was obtained as a white solid.

LCMS: RT=2.641 min, MS cal.: 593.1/595.1, [M+H]⁺=594.2/596.2

HPLC: RT=11.207 min, purity: 99.56%

¹H NMR (400 MHz, DMSO-d6) δ=8.57 (d, J=1.6 Hz, 1H), 8.06 (s, 1H), 7.63 (t, J=74.4 Hz, 1H), 7.57 (t, J=8.0 Hz, 1H), 7.51 (s, 1H), 7.48 (dd, J=1.6, 9.6 Hz, 1H), 7.31 (dd, J=1.2, 8.0 Hz, 1H), 5.36 (s, 2H), 3.96 (s, 3H), 3.86 (s, 2H), 2.94 (d, J=10.8 Hz, 3H), 2.24 (dt, J=3.6, 10.8 Hz, 2H), 1.83-1.69 (m, 4H)

### Example A114—Preparation of Compound 186. 2-((4-(6-((4-Chloro-2-fluorobenzyl)oxy)pyridin-2-yl)piperidin-1-yl)methyl)-4-((2-fluoroethyl) amino)-1-methyl-1H-benzo[d]imidazole-6-carboxylic acid (Compound 186) (12)

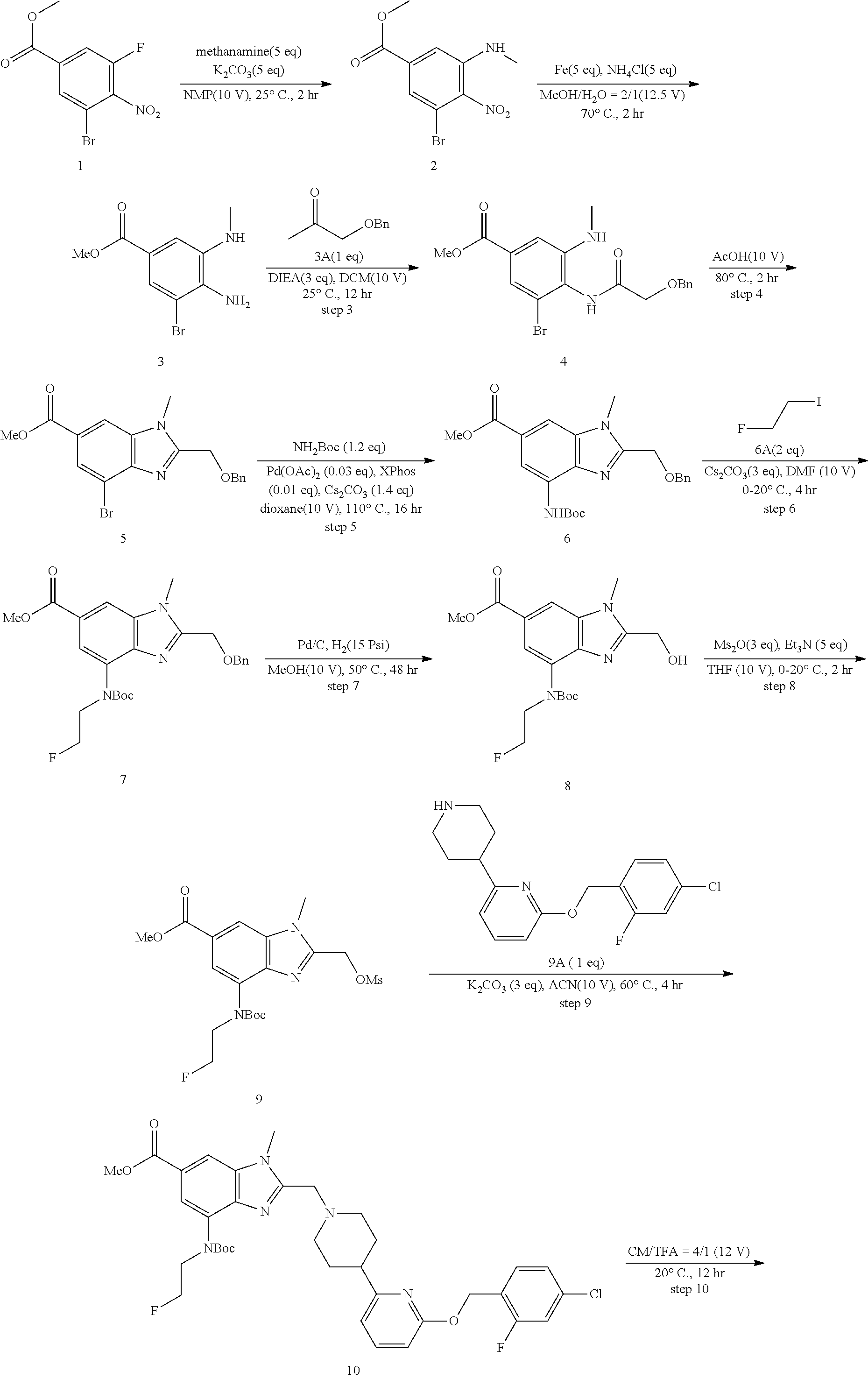

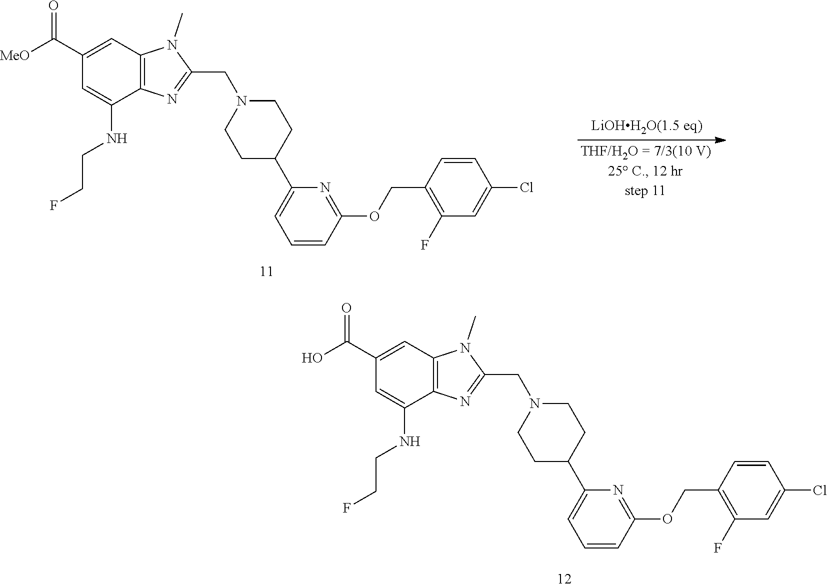 [SMILES: COC(=O)c1cc(NCCF)c2nc(CN3CCC(c4cccc(OCc5ccc(Cl)cc5F)n4)CC3)n(C)c2c1 ; Cn1c(CN2CCC(c3cccc(OCc4ccc(Cl)cc4F)n3)CC2)nc2c(NCCF)cc(C(=O)O)cc21]

### General Procedure for Preparation of Intermediate 2:

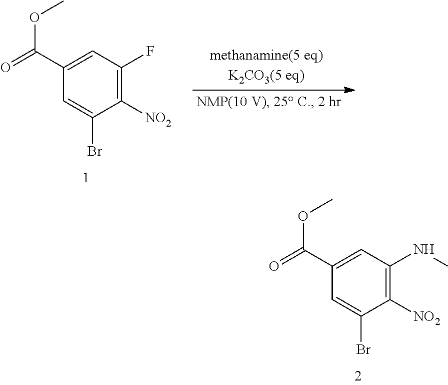 [SMILES: COC(=O)c1cc(F)c([N+](=O)[O-])c(Br)c1 ; CNc1cc(C(=O)OC)cc(Br)c1[N+](=O)[O-]]

Equip a 100 mL three-necked round bottom flask, thermometer, N₂ balloon. NMP (40 mL) was charged to the 100 mL three-necked round bottom flask, then 1 (4 g, 14.39 mmol, 1 eq), K₂CO₃ (9.94 g, 71.93 mmol, 5 eq) was added to the mixture at 25° C. methanamine (2 M, 14.39 mL, 2 eq) was added dropwise to the reaction mixture at 25° C. After the addition, the mixture was stirred at 25° C. for 2 hr. The reaction was monitored by TLC (Petroleum ether:Ethyl acetate=3:1, R_{f}=0.63). After 2 hr, FA (1 M) was added reaction mixture to adjust pH=5. The reaction mixture was extracted with EtOAc 90 mL (30 mL*3). The combined organic layers were washed with brine 40 mL (20 mL*2), dried over Na₂SO₄, filtered and concentrated under reduced pressure to give a residue. The residue was purified by column chromatography (SiO₂, Petroleum ether/Ethyl acetate=100/1 to 100/30). 2 (4 g, 13.84 mmol, 96.18% yield) was obtained as a yellow solid.

¹H NMR (400 MHz, CHCl3-d) δ=7.59 (d, J=1.6 Hz, 1H), 7.42 (d, J=1.6 Hz, 1H), 3.95 (s, 3H), 2.98 (s, 3H)

### General Procedure for Preparation of Intermediate 3:

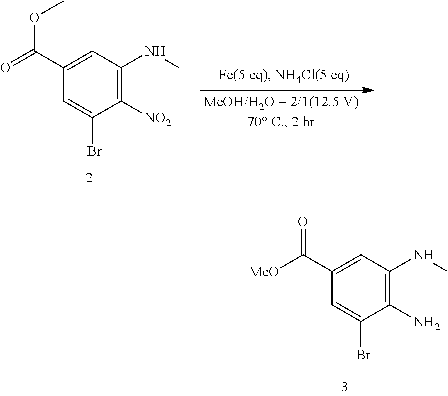 [SMILES: CNc1cc(C(=O)OC)cc(Br)c1[N+](=O)[O-] ; CNc1cc(C(=O)OC)cc(Br)c1N]

Equip a 250 mL three-necked round bottom flask and thermometer, N₂ balloon. MeOH (30 mL) was charged to the 250 mL flask, then 2 (3.6 g, 12.45 mmol, 1 eq) was added at 25° C. under N₂. At 25° C., NH₄Cl (3.33 g, 62.27 mmol, 5 eq) in H₂O (15 mL) was added to the reaction mixture at 25° C. under N₂ atmosphere. The mixture was heated to 40° C. Fe (3.48 g, 62.27 mmol, 5 eq) was added in portions to the reaction mixture at 40° C. under N₂ atmosphere. After the addition, the suspension was degassed and purged with N₂ for 3 times, and then the mixture was stirred under N₂ atmosphere at 70° C. for 2 hr. LCMS (ET84080-11-p1e, RT=0.406 min) showed 2 was consumed completely and one main peak with desired mass was detected. After 2 hr, the suspension was filtered through a pad of Celite and filter cake was washed with MeOH 60 mL (20 mL×3). Then the organic layer was concentrated under reduced pressure to give a residue. The residue was dissolved with EtOAc 50 mL, the organic layer was washed with H₂O 60 mL (20 mL×3), brine 30 mL (15 mL*2), dried over Na₂SO₄, filtered and concentrated under reduced pressure to give a residue. The was used directly to next step. 3 (3.2 g, 12.35 mmol, 99.18% yield) was obtained as a yellow solid.

LCMS: RT=0.406 min, MS cal.: 258.0/260.0, [M+H]⁺=259.0/261.8

¹H NMR (400 MHz, CHCl3-d) δ=7.74 (d, J=1.8 Hz, 1H), 7.29 (d, J=1.8 Hz, 1H), 3.88 (s, 3H), 2.91 (s, 3H)

### General Procedure for Preparation of Intermediate 4:

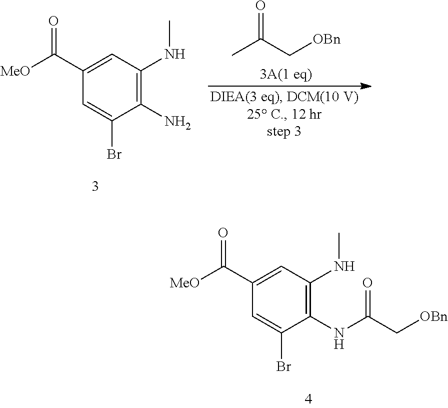 [SMILES: CNc1cc(C(=O)OC)cc(Br)c1N ; CC(=O)COCc1ccccc1 ; CNc1cc(C(=O)OC)cc(Br)c1NC(=O)COCc1ccccc1]

To a solution of 3 (4 g, 15.44 mmol, 1 eq) and DIEA (5.99 g, 46.31 mmol, 8.07 mL, 3 eq) in DCM (40 mL) was added a solution of 3A (2.85 g, 15.44 mmol, 2.40 mL, 1 eq) in DCM (10 mL). The mixture was stirred at 25° C. for 2 hrs. LC-MS showed Reactant 1 was consumed completely and one main peak with desired mass was detected. The reaction mixture was concentrated under reduced pressure to remove DCM. Then the residue was diluted with EtOAc 50 mL and H₂O 50 mL at 25° C., and then extracted with EtOAc 120 mL (40 mL*3). The combined organic layers were washed with 1M FA 15 mL, aq. NaHCO₃20 mL*2, brine 20 mL, dried over Na₂SO₄, filtered and concentrated under reduced pressure to give a residue. The crude product 4 (7.0 g, crude) as a brown oil was used into the next step without further purification.

### General Procedure for Preparation of Intermediate 5:

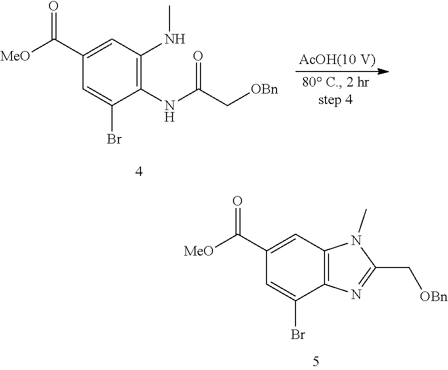 [SMILES: CNc1cc(C(=O)OC)cc(Br)c1NC(=O)COCc1ccccc1 ; COC(=O)c1cc(Br)c2nc(COCc3ccccc3)n(C)c2c1]

A mixture of 4 (7 g, 16.62 mmol, 1 eq) in AcOH (70 mL) was degassed and purged with N₂ for 3 times, and then the mixture was stirred at 80° C. for 2 hrs under N₂ atmosphere. LC-MS showed 4 was consumed completely and one main peak with desired mass was detected. The reaction mixture was concentrated under reduced pressure to remove AcOH. The residue was diluted with EtOAc 50 mL and then the mixture was added aq. Na₂CO₃ 20 mL to adjust pH=9, extracted with EtOAc 90 mL (30 mL*3), washed with brine 40 mL, dried with Na₂SO₄, filtered and concentrated under reduced pressure to give a residue. The residue was purified by column chromatography (SiO₂, Petroleum ether/Ethyl acetate=1/0 to 2/1). 5 (5.5 g, 14.13 mmol, 85.04% yield) was obtained as a gray solid.

LCMS: RT=0.507 min, MS cal.: 388.0, [M+H]⁺=389.0

¹H NMR (400 MHz, CHCl3-d) δ=8.19 (d, J=1.2 Hz, 1H), 8.06 (d, J=1.2 Hz, 1H), 7.38-7.29 (m, 5H), 4.93 (s, 2H), 4.62 (s, 2H), 3.97 (s, 3H), 3.90-3.87 (m, 3H)

### General Procedure for Preparation of Intermediate 6:

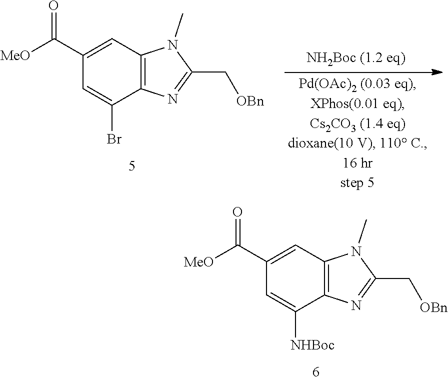 [SMILES: COC(=O)c1cc(Br)c2nc(COCc3ccccc3)n(C)c2c1 ; COC(=O)c1cc(NC(=O)OC(C)(C)C)c2nc(COCc3ccccc3)n(C)c2c1]

A mixture of 5 (1 g, 2.57 mmol, 1 eq), NH₂Boc (601.92 mg, 5.14 mmol, 2 eq), Cs₂CO₃ (1.67 g, 5.14 mmol, 2 eq), Pd(OAc)₂ (28.84 mg, 128.45 μmol, 0.05 eq) and XPhos (244.95 mg, 513.82 μmol, 0.2 eq) in dioxane (10 mL) was degassed and purged with N₂ for 3 times, and then the mixture was stirred at 110° C. for 16 hrs under N₂ atmosphere. TLC indicated 5 was consumed completely and one new spot formed. The reaction was clean according to TLC (Petroleum ether:Ethyl acetate=2:1, Product R_{f}=0.52). The residue mixture was diluted with H₂O 50 mL and extracted with EtOAc (50 mL*2). The combined organic layers were washed with brine 50 mL, dried over Na₂SO₄, filtered and concentrated under reduced pressure to give a residue. The residue was purified by column chromatography (SiO₂, Petroleum ether/Ethyl acetate=100/1 to 7/3, TLC-Petroleum ether/Ethyl acetate-2/1, Product Rr-0.52). 6 (940 mg, 2.07 mmol, 80.54% yield) was obtained as a colorless oil.

LCMS: RT=0.537 min, MS cal.: 425.20, [M+H]⁺=426.2

¹H NMR (400 MHz, CHLOROFORM-d) δ=8.60 (s, 1H), 7.81 (s, 1H), 7.68 (br s, 1H), 7.39-7.32 (m, 5H), 4.83 (s, 2H), 4.61 (s, 2H), 3.95 (s, 3H), 3.85 (s, 3H), 1.57 (s, 9H)

### General Procedure for Preparation of Intermediate 7:

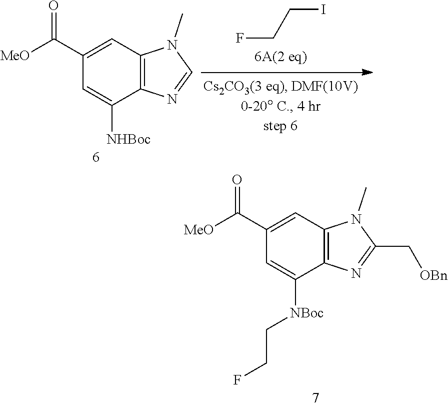

A mixture of 6 (900 mg, 2.12 mmol, 1 eq) in DMF (9 mL) was degassed and purged with N₂ for 3 times. The mixture was Cooled to 0° C. and Cs₂CO₃ (2.07 g, 6.35 mmol, 3 eq) was added to the reaction mixture at 0° C. Then the mixture was stirred at 20° C. for 0.5 hrs under N₂ atmosphere. And then 6A (735.93 mg, 4.23 mmol, 2 eq) in DMF (1 mL) was added dropwise to the reaction mixture. After the addition, the mixture was stirred at 20° C. for 12 hr. TLC indicated 6 was consumed completely and one new spot formed. The reaction was clean according to TLC (Petroleum ether:Ethyl acetate=2:1, Product R_{f}=0.24). The residue mixture was diluted with H₂O 100 mL and extracted with EtOAc (30 mL*3). The combined organic layers were washed with brine (30 mL*4), dried over Na₂SO₄, filtered, and concentrated under reduced pressure to give a residue. The residue was purified by column chromatography (SiO₂, Petroleum ether/Ethyl acetate=100/1 to 1/1, TLC-Petroleum ether/Ethyl acetate=2/1, Product R_{f}=0.24). 7 (760 mg, 1.54 mmol, 72.65% yield, 95.34% purity) was obtained as a colorless oil.

LCMS: RT=0.761 min, MS cal.: 471.22, [M+H]⁺=472.3

¹H NMR (400 MHz, CHLOROFORM-d) δ=8.01 (s, 1H), 7.91 (br s, 1H), 7.38-7.30 (m, 5H), 4.88 (s, 2H), 4.67-4.53 (dt, J=47.2, 5.6 Hz, 2H), 4.62 (s, 2H), 4.29-4.18 (dt, J=23.6, 5.2 Hz, 2H), 3.96 (s, 3H), 3.88 (s, 3H), 1.42 (br s, 9H)

### General Procedure for Preparation of Intermediate 8

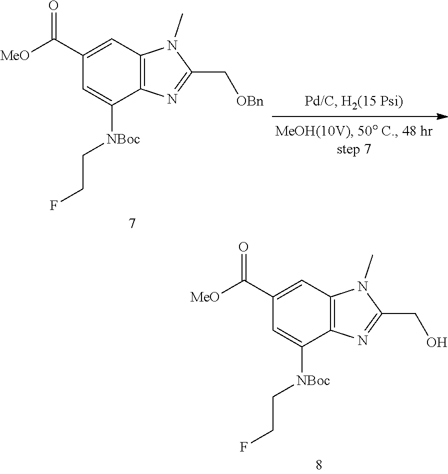 [SMILES: COC(=O)c1cc(N(CCF)C(=O)OC(C)(C)C)c2nc(COCc3ccccc3)n(C)c2c1 ; COC(=O)c1cc(N(CCF)C(=O)OC(C)(C)C)c2nc(CO)n(C)c2c1]

A mixture of 7 (660 mg, 1.40 mmol, 1 eq,), Pd/C (330.00 mg) in MeOH (6.6 mL) was degassed and purged with H₂ for 3 times, and then the mixture was stirred at 50° C. for 48 hrs under H₂ (15 Psi) atmosphere. TLC indicated ˜10% of 7 was remained, and one major new spot with larger polarity was detected (Petroleum ether:Ethyl acetate=1:1, product Rf=0.13). The reaction mixture was filtered, and filtrate was concentrated under reduced pressure to give a residue. The crude product was triturated with EtOAc 10 mL at 20° C. for 10 min and then filtered. The filtrate was concentrated under reduced pressure to give a residue. 8 (320 mg, 798.08 μmol, 57.02% yield) was obtained as a white solid.

LCMS: RT=0.592 min, MS cal.: 381.17, [M+H]⁺=382.2

¹H NMR (400 MHz, CHLOROFORM-d) δ=8.02 (s, 1H), 7.90 (br s, 1H), 4.91 (br d, J=5.2 Hz, 2H), 4.68-4.48 (dt, J=47.2, 5.2 Hz, 2H), 4.22-4.11 (dt, J=23.6, 5.2 Hz, 2H), 4.04 (br t, J=5.6 Hz, 1H), 3.96 (s, 3H), 3.86 (s, 3H), 1.41 (br s, 9H)

### General Procedure for Preparation of Intermediate 9:

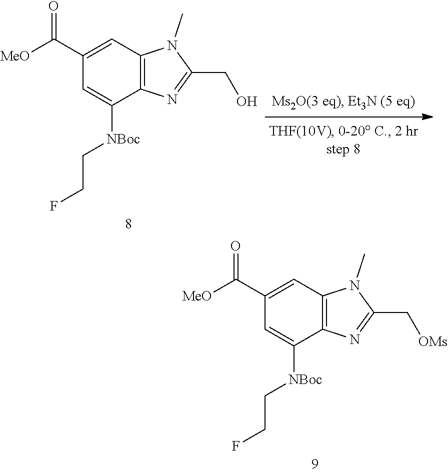 [SMILES: COC(=O)c1cc(N(CCF)C(=O)OC(C)(C)C)c2nc(CO)n(C)c2c1 ; COC(=O)c1cc(N(CCF)C(=O)OC(C)(C)C)c2nc(COS(C)(=O)=O)n(C)c2c1]

A mixture of 8 (100 mg, 262.19 μmol, 1 eq), Et₃N (132.66 mg, 1.31 mmol, 182.47 μL, 5 eq) in THF (1 mL) was degassed and purged with N₂ for 3 times. The mixture was Cooled to 0° C. and Ms₂O (137.02 mg, 786.58 μmol, 3 eq) was added, dropwise, keeping the internal temperature below 10° C. Then the mixture was stirred at 20° C. for 2 hrs under N₂ atmosphere. TLC indicated 8 was consumed completely and one new spot formed. The reaction was clean according to TLC (Petroleum ether:Ethyl acetate=1:1, Product R_{f}=0.50). The residue mixture was diluted with H₂O 10 mL and extracted with DCM (10 mL*3). The combined organic layers were washed with brine 10 mL, dried over Na₂SO₄, filtered, and concentrated under reduced pressure to give a residue. 9 (120 mg, crude) was obtained as a colorless oil.

LCMS: RT=0.703 min, MS cal.: 459.15, [M+H]⁺−55=404.1

¹H NMR (400 MHz, CHLOROFORM-d) δ=8.07 (s, 1H), 7.95 (br s, 1H), 5.54 (s, 2H), 4.68-4.52 (dt, J=47.2, 5.2 Hz, 2H), 4.25-4.13 (dt, J=24.0, 5.2 Hz, 2H), 3.97 (s, 3H), 3.95-3.92 (m, 3H), 3.12 (s, 3H), 1.47-1.38 (m, 9H)

### General Procedure for Preparation of Intermediate 10:

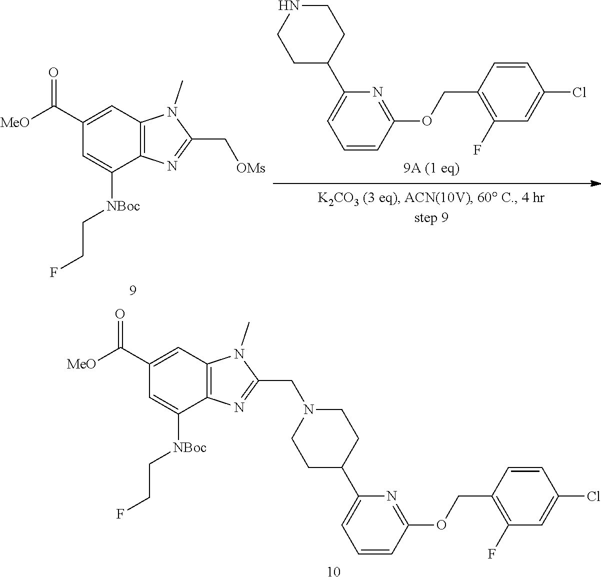 [SMILES: COC(=O)c1cc(N(CCF)C(=O)OC(C)(C)C)c2nc(COS(C)(=O)=O)n(C)c2c1 ; Fc1cc(Cl)ccc1COc1cccc(C2CCNCC2)n1 ; COC(=O)c1cc(N(CCF)C(=O)OC(C)(C)C)c2nc(CN3CCC(c4cccc(OCc5ccc(Cl)cc5F)n4)CC3)n(C)c2c1]

A mixture of 9 (120 mg, 261.16 μmol, 1 eq,), 9A (83.78 mg, 261.16 μmol, 1 eq), K₂CO₃ (108.28 mg, 783.48 μmol, 3 eq) in ACN (1.2 mL) was degassed and purged with N₂ for 3 times, and then the mixture was stirred at 60° C. for 4 hrs under N₂ atmosphere. LC-MS showed 9 was consumed completely and one main peak with desired mass was detected. The residue mixture was diluted with H₂O 10 mL and extracted with EtOAc (10 mL*3). The combined organic layers were washed with brine 10 mL, dried over Na₂SO₄, filtered and concentrated under reduced pressure to give a residue. The residue was purified by column chromatography (SiO₂, Petroleum ether/Ethyl acetate=100/1 to 1/1, TLC-Petroleum ether/Ethyl acetate=1/1, Product R (=0.43). 10 (160 mg, 233.86 μmol, 89.55% yield) was obtained as a colorless oil.

LCMS: RT=0.810 min, MS cal.: 683.27, [M+H]⁺=684.3

¹H NMR (400 MHz, CHLOROFORM-d) δ=8.02 (s, 1H), 7.89 (br s, 1H), 7.50 (t, J=7.6 Hz, 1H), 7.44 (t, J=8.0 Hz, 1H), 7.15-7.07 (m, 2H), 6.74 (d, J=7.2 Hz, 1H), 6.61 (d, J=8.4 Hz, 1H), 5.41 (s, 2H), 4.70-4.51 (dt, J=47.2, 5.2 Hz, 2H), 4.30-4.17 (dt, J=23.6, 5.2 Hz, 2H), 3.97 (d, J=10.4 Hz, 6H), 3.89 (s, 2H), 2.98 (br d, J=10.4 Hz, 2H), 2.68-2.56 (m, 1H), 2.30 (br t, J=10.4 Hz, 2H), 1.93-1.80 (m, 4H), 1.42 (br s, 9H)

### General Procedure for Preparation of Intermediate 11:

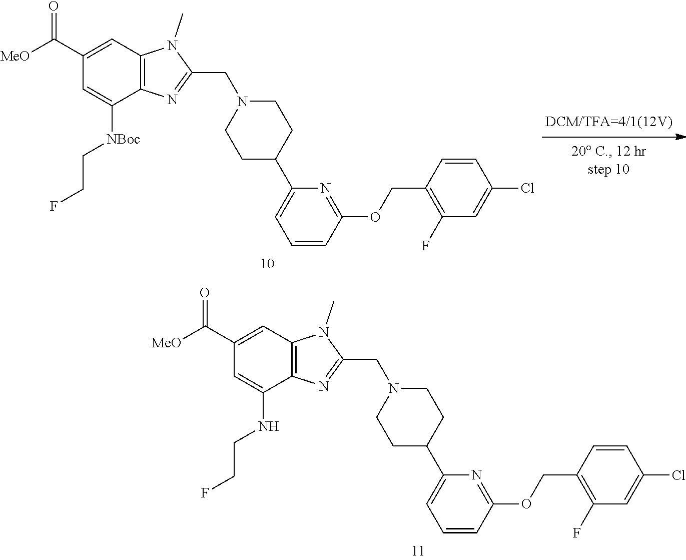 [SMILES: COC(=O)c1cc(N(CCF)C(=O)OC(C)(C)C)c2nc(CN3CCC(c4cccc(OCc5ccc(Cl)cc5F)n4)CC3)n(C)c2c1 ; COC(=O)c1cc(NCCF)c2nc(CN3CCC(c4cccc(OCc5ccc(Cl)cc5F)n4)CC3)n(C)c2c1]

A mixture of 10 (160 mg, 233.86 μmol, 1 eq,), TFA (583.30 mg, 5.12 mmol, 0.38 mL, 21.87 eq) in DCM (1.54 mL) was degassed and purged with N₂ for 3 times, and then the mixture was stirred at 20° C. for 2 hrs under N₂ atmosphere. LC-MS showed 10 was consumed completely and one main peak with desired mass was detected. The reaction mixture was adjusted to pH=12 with a.q. Na₂CO₃ and extracted with EtOAc (10 mL*3). The combined organic layers were washed with brine 10 mL, dried over Na₂SO₄, filtered, and concentrated under reduced pressure to give a residue. 11 (110 mg, crude) was obtained as a white oil.

LCMS: RT=1.700 min, MS cal.: 583.22, [M+H]⁺=584.1

¹H NMR (400 MHz, CHLOROFORM-d) δ=7.54 (d, J=1.2 Hz, 1H), 7.52-7.48 (m, 1H), 7.44 (t, J=8.0 Hz, 1H), 7.14-7.07 (m, 3H), 6.74 (d, J=7.2 Hz, 1H), 6.61 (d, J=8.0 Hz, 1H), 5.41 (s, 2H), 5.25-5.15 (m, 1H), 4.80-4.62 (dt, J=47.2, 4.8 Hz, 2H), 3.94 (d, J=5.6 Hz, 6H), 3.85 (br s, 2H), 3.75-3.63 (dq, J=26.0, 5.2 Hz, 2H), 3.00 (br d, J=9.6 Hz, 2H), 2.69-2.56 (m, 1H), 2.28 (br s, 2H), 1.93-1.81 (m, 4H)

### General Procedure for Preparation of Compound 186 (12):

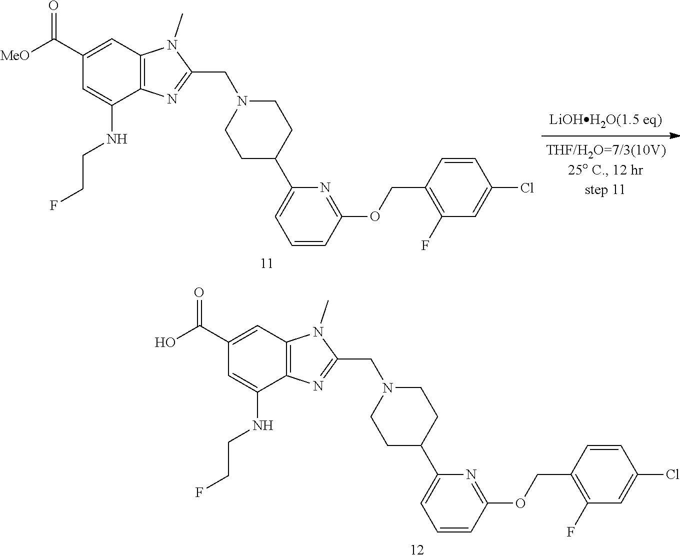 [SMILES: COC(=O)c1cc(NCCF)c2nc(CN3CCC(c4cccc(OCc5ccc(Cl)cc5F)n4)CC3)n(C)c2c1 ; Cn1c(CN2CCC(c3cccc(OCc4ccc(Cl)cc4F)n3)CC2)nc2c(NCCF)cc(C(=O)O)cc21]

A mixture of 11 (110 mg, 188.34 μmol, 1 eq), LiOH·H₂O (11.86 mg, 282.51 μmol, 1.5 eq) in THF (0.77 mL) and H₂O (0.33 mL) was degassed and purged with N₂ for 3 times, and then the mixture was stirred at 25° C. for 72 hrs under N₂ atmosphere. LC-MS showed ˜32% of Reactant 1 remained. Several new peaks were shown on LC-MS and ˜60% of desired Compound was detected. The crude reaction mixture on notebook page ET62904-697 was combined to ET62904-700 for workup. The reaction mixture was diluted with ACN 0.5 mL. The liquid was purified by prep-HPLC (column: Waters Xbridge Prep OBD C₁₈ 150*40 mm*10 um; mobile phase: [H₂O (10 mM NH₄HCO₃)-ACN]; gradient: 20%-70% B over 8.0 min). 2-((4-(6-((4-Chloro-2-fluorobenzyl)oxy)pyridin-2-yl)piperidin-1-yl)methyl)-4-((2-fluoroethyl) amino)-1-methyl-1H-benzo[d]imidazole-6-carboxylic acid (28.59 mg, 50.16 μmol, 26.63% purity) was obtained as a white solid.

LCMS: RT=1.210 min, MS cal.: 569.20, [M+H]⁺=570.1

¹H NMR (400 MHz, DMSO-d6) δ=12.80-12.19 (m, 1H), 7.62 (t, J=7.6 Hz, 1H), 7.55 (t, J=8.0 Hz, 1H), 7.49-7.40 (m, 2H), 7.28 (dd, J=1.6, 8.2 Hz, 1H), 6.92 (s, 1H), 6.86 (d, J=7.2 Hz, 1H), 6.66 (d, J=8.0 Hz, 1H), 5.90 (br t, J=6.0 Hz, 1H), 5.35 (s, 2H), 4.70-4.56 (dt, J=47.6, 5.2 Hz, 2H), 3.87 (s, 3H), 3.79 (s, 2H), 3.64-3.54 (dq, J=25.6, 4.8 Hz, 2H), 2.93 (br d, J=10.8 Hz, 2H), 2.63-2.53 (m, 1H), 2.18 (br t, J=11.2 Hz, 2H), 1.82-1.64 (m, 4H)

### Example A115—Preparation of Compound 187. 2-((4-(6-((4-Chloro-2-fluorobenzyl)oxy)pyridin-2-yl)piperidin-1-yl)methyl)-4-methoxy-1-methyl-1H-benzo[d]imidazole-5-carboxylic acid (Compound 187) (7)

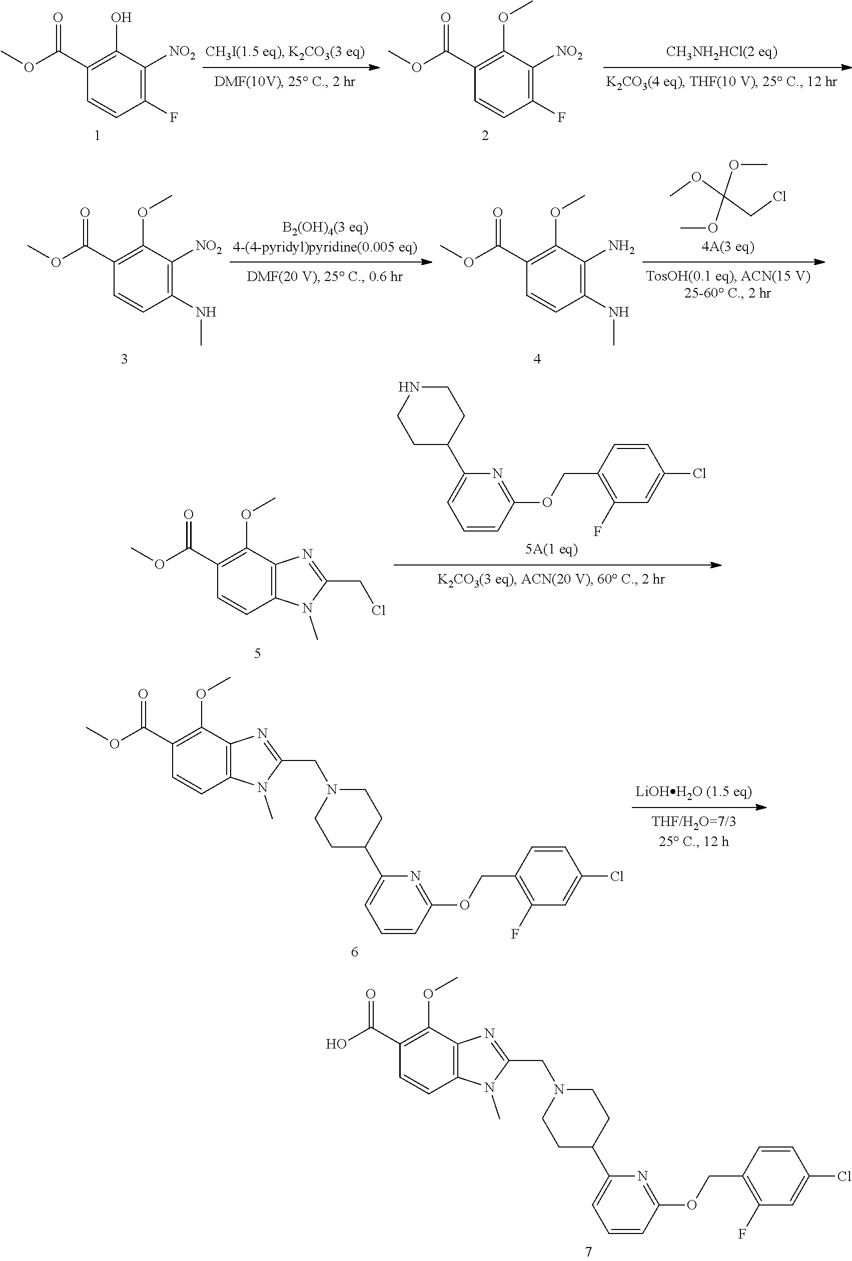

### General Procedure for Preparation of Intermediate 2:

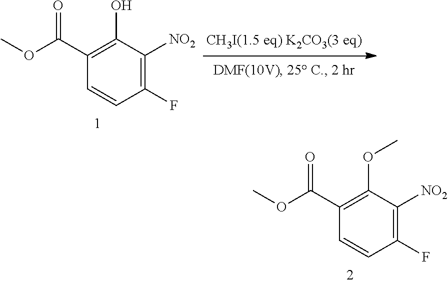 [SMILES: COC(=O)c1ccc(F)c([N+](=O)[O-])c1O ; COC(=O)c1ccc(F)c([N+](=O)[O-])c1OC]

Equip a 100 mL single round bottom flask, addition funnel and thermometer, N₂ balloon. DMF (20 mL) was charged to the three-necked round bottom flask, then 1 (2 g, 9.30 mmol, 1 eq) and K₂CO₃ (3.85 g, 27.89 mmol, 3 eq) was added to the mixture at 25° C. At 25° C. inner temperature, CH₃I (1.98 g, 13.94 mmol, 868.11 μL, 1.5 eq) was added dropwise to the reaction mixture at 25° C. After the addition, the mixture was stirred at 25° C. for 2 hr. TLC (PE:EA=3:1) indicated 1 was consumed completely and one new spot formed. After 2 hr, the reaction mixture was added to 50 mL at 0° C. The mixture was extracted by DCM (10 mL*3). Then organic phase was combined and washed by H₂O (50 mL), aq. brine (50 mL), dried with Na₂SO₄, filtered. The organic phase was concentrated under reduced pressure at 45° C. to give a residue. The residue was purified by column chromatography (SiO₂, Petroleum ether/Ethyl acetate=10/1 to 0/1). 2 (1.6 g, 6.98 mmol, 75.10% yield) was obtained as a white solid.

¹H NMR (400 MHz, CDCl₃) δ=8.10-8.01 (m, 1H), 7.13-7.03 (m, 1H), 4.01 (s, 3H), 3.98-3.93 (m, 3H)

### General Procedure for Preparation of Intermediate 3:

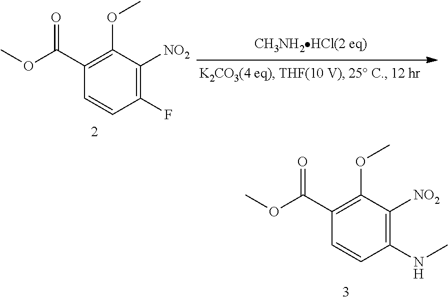

A mixture of 2 (600 mg, 2.62 mmol, 1 eq), K₂CO₃ (1.45 g, 10.47 mmol, 4 eq), CH₃NH₂·HCl (353.56 mg, 5.24 mmol, 2 eq) and in THF (6 mL) was degassed and purged with N₂ for 3 times, and then the mixture was stirred at 25° C. for 12 hr under N₂ atmosphere. LC-MS (RT=1.099 min) showed 2 was consumed completely and one main peak with desired mass was detected. The reaction mixture was diluted with H₂O 20 mL and extracted with EtOAc 60 mL (20 mL*3). The combined organic layers were washed with brine 15 mL, dried over Na₂SO₄, filtered, and concentrated under reduced pressure to give a residue. 3 (610 mg, 2.54 mmol, 96.99% yield) was obtained as a yellow solid.

LCMS: RT=1.368 min, MS cal.: 240.9, [M+1]⁺=241.9

¹H NMR (400 MHz, CDCl₃) δ=7.94 (d, J=9.2 Hz, 1H), 6.54 (d, J=9.2 Hz, 1H), 6.08 (br s, 1H), 3.97 (s, 3H), 3.90 (s, 3H), 2.97 (s, 3H)

### General Procedure for Preparation of Intermediate 4:

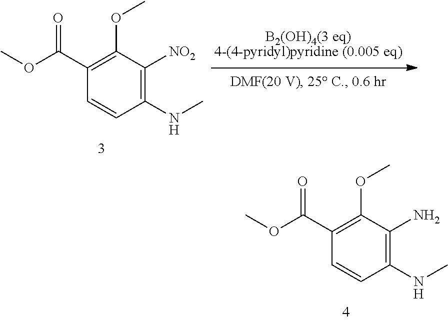

Equip a 100 mL three-necked round bottom flask, thermometer, N₂ balloon. DMF (7 mL) was charged to the 100 mL one-necked round bottom flask, then 3 (600 mg, 2.50 mmol, 1 eq) and 4-(4-pyridyl)pyridine (1.95 mg, 12.49 μmol, 0.005 eq) was added to the mixture at 25° C. At 25° C. (inner temperature), B₂(OH)₄ (671.79 mg, 7.49 mmol, 3 eq) was added dropwise to the reaction mixture. After the addition, the mixture was stirred at 25° C. for 0.6 hr. LC-MS (product, RT=0.877 min) showed 3 was consumed completely and one main peak with desired mass was detected. The reaction mixture was diluted with H₂O 30 mL and extracted with EtOAc 75 mL (25 mL*3). The combined organic layers were washed with brine 15 mL, dried over Na₂SO₄, filtered and concentrated under reduced pressure to give a residue. The residue was purified by column chromatography (SiO₂, Petroleum ether/Ethyl acetate=10/1 to 1/1). 4 (330 mg, 1.57 mmol, 62.84% yield) was obtained as a white solid.

LCMS: RT=0.877 min, MS cal.: 210.23, [M+1]⁺=211.0

¹H NMR (400 MHz, CDCl₃) δ=7.51 (br d, J=8.6 Hz, 1H), 6.44 (d, J=8.6 Hz, 1H), 3.87 (d, J=8.1 Hz, 6H), 3.62-3.27 (m, 3H), 2.93 (s, 3H)

### General Procedure for Preparation of Intermediate 5:

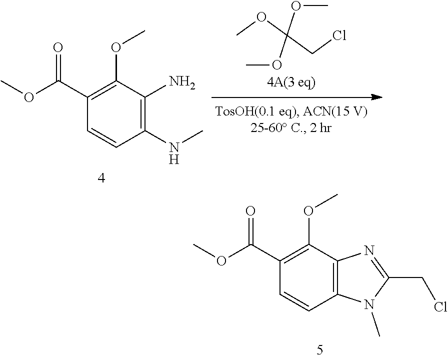

A mixture of 4 (727.99 mg, 4.71 mmol, 634.69 μL, 3 eq), 4A (727.99 mg, 4.71 mmol, 634.69 μL, 3 eq), TosOH (27.03 mg, 156.97 μmol, 0.1 eq) in ACN (5 mL) was degassed and purged with N₂ for 3 times, and then the mixture was stirred at 60° C. for 2 hr under N₂ atmosphere. LC-MS (product, RT=1.222 min) showed 4 was consumed completely and one main peak with desired mass was detected. The reaction mixture was concentrated under reduced pressure to give a residue. 5 (420 mg, 1.56 mmol, 99.58% yield) was obtained as a blue liquid.

LCMS: RT=1.454 min, MS cal.: 353.07, [M+1]⁺=354.1

### General Procedure for Preparation of Intermediate 6:

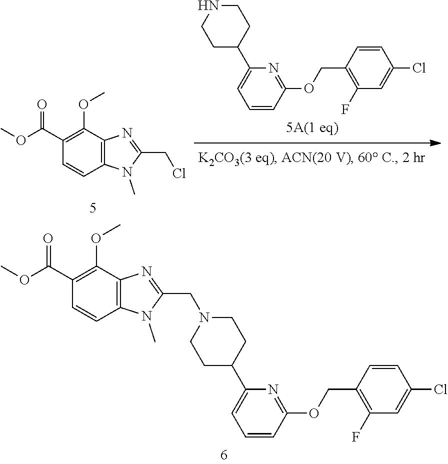

A mixture of 5 (270 mg, 1.00 mmol, 1 eq), 5A (322.35 mg, 1.00 mmol, 1 eq), K₂CO₃ (416.63 mg, 3.01 mmol, 3 eq) in ACN (6 mL) was degassed and purged with N₂ for 3 times, and then the mixture was stirred at 60° C. for 2 hr under N₂ atmosphere. LC-MS (product RT=1.586 min) showed 5 was consumed completely and one main peak with desired mass was detected. The reaction mixture was diluted with H₂O 15 mL and extracted with EtOAc 45 mL (15 mL*3). The combined organic layers were washed with brine 15 mL, dried over Na₂SO₄, filtered, and concentrated under reduced pressure to give a residue. The residue was purified by column chromatography (SiO₂, Petroleum ether/Ethyl acetate=10/1 to 0/1). 6 (450 mg, 813.71 μmol, 80.98% yield) was obtained as a yellow oil.

LCMS: RT=1.586 min, MS cal.: 553.03, [M+1]⁺=554.3

¹H NMR (400 MHz, DMSO-d6) δ=7.77 (d, J=8.6 Hz, 1H), 7.57-7.38 (m, 2H), 7.15-7.08 (m, 2H), 7.03 (d, J=8.6 Hz, 1H), 6.74 (d, J=7.2 Hz, 1H), 6.61 (d, J=8.2 Hz, 1H), 5.41 (s, 2H), 4.40 (s, 3H), 3.93 (s, 6H), 3.87 (br s, 1H), 2.99 (br dd, J=2.0, 3.2 Hz, 2H), 2.62 (br s, 1H), 2.30 (br s, 2H), 1.97-1.75 (m, 4H), 1.61 (br s, 2H)

### General Procedure for Preparation of Compound 187 (7):

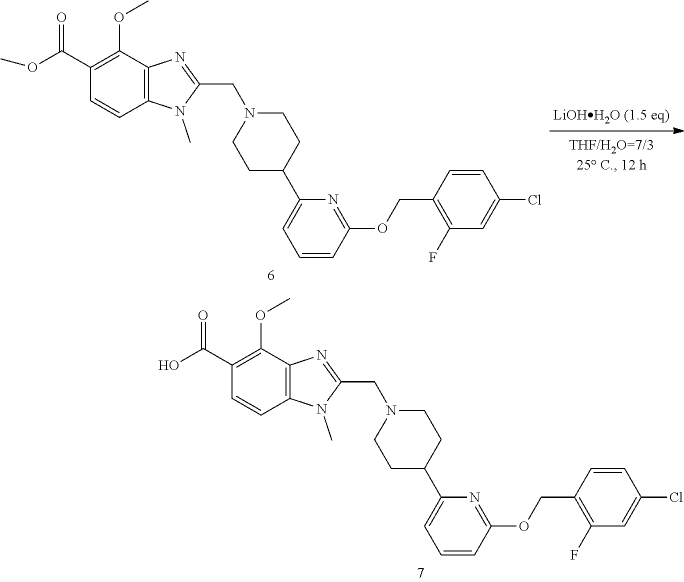

A mixture of 6 (80 mg, 144.66 μmol, 1 eq), LiOH·H₂O (9.11 mg, 216.99 μmol, 1.5 eq) in THF (0.7 mL) and H₂O (0.3 mL) was degassed and purged with N₂ for 3 times, and then the mixture was stirred at 25° C. for 12 hr under N₂ atmosphere. LC-MS (ET83888-82, product RT=1.198 min) showed 28% of 6 remained. Several new peaks were shown on LC-MS and 67% of desired Compound was detected. The reaction was purified directly. The residue was purified by prep-HPLC (column: Waters Xbridge Prep OBD C18 150*40 mm*10 um; mobile phase: [H₂O (10 mM NH₄HCO₃)-ACN]; gradient: 15%-65% B over 8.0 min). 2-((4-(6-((4-Chloro-2-fluorobenzyl)oxy)pyridin-2-yl)piperidin-1-yl)methyl)-4-methoxy-1-methyl-1H-benzo[d]imidazole-5-carboxylic acid (26.29 mg, 48.78 μmol, 33.72% yield) was obtained as a white solid.

LCMS: RT=2.389 min, MS cal.: 539.00, [M+1]⁺=540.2

HPLC: RT=11.37 min, purity: 100%

¹H NMR (400 MHz, MEOD) δ=7.79 (d, J=8.8 Hz, 1H), 7.57 (t, J=8.0 Hz, 1H), 7.49 (t, J=8.4 Hz, 1H), 7.26 (d, J=8.8 Hz, 1H), 7.22-7.14 (m, 2H), 6.82 (d, J=7.2 Hz, 1H), 6.63 (d, J=8.0 Hz, 1H), 5.40 (s, 2H), 4.29 (s, 3H), 3.96 (s, 3H), 3.94 (s, 2H), 3.08 (br d, J=11.2 Hz, 2H), 2.72-2.60 (m, 1H), 2.43-2.32 (m, 2H), 1.92-1.84 (m, 4)

### Example A116—Preparation of Compound 188. 2-((4-(6-((4-Chloro-2-fluorobenzyl)oxy)pyridin-2-yl)piperidin-1-yl)methyl)-4-((1-fluorocyclopropyl)methoxy)-1-methyl-1H-benzo[d]imidazole-6-carboxylic acid (Compound 188) (3)

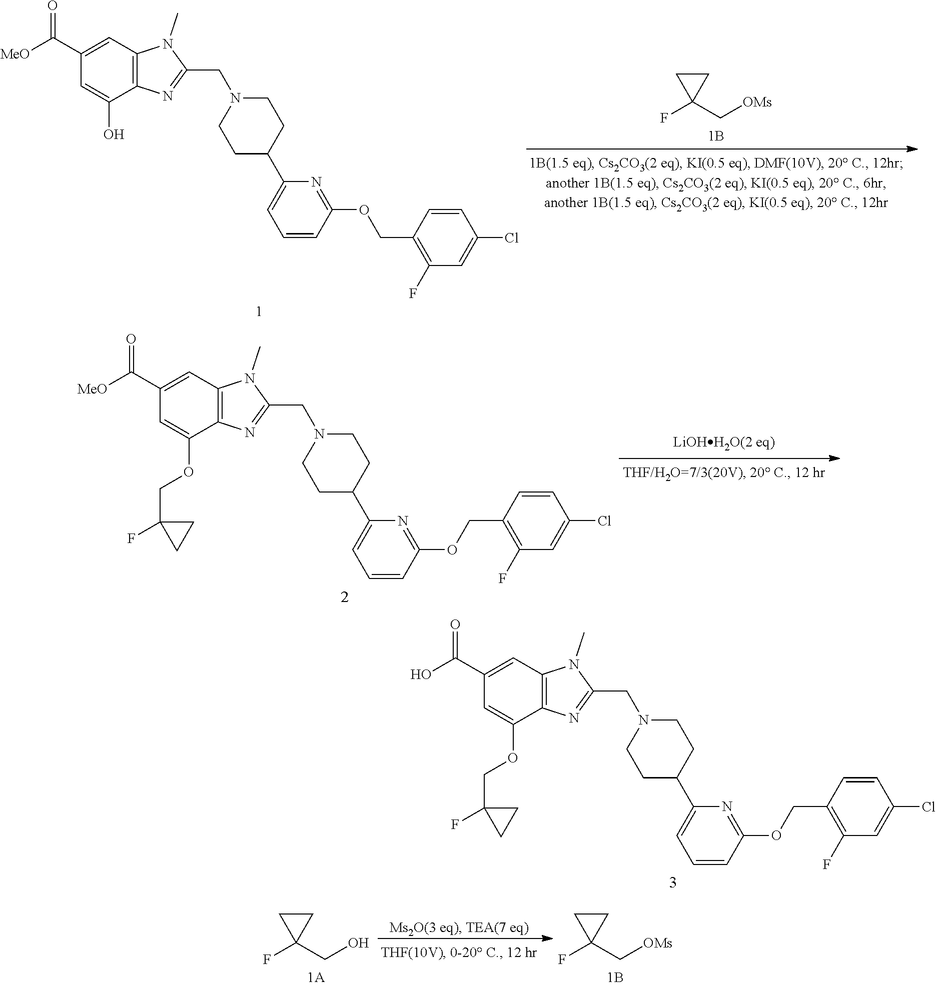 [SMILES: COC(=O)c1cc(O)c2nc(CN3CCC(c4cccc(OCc5ccc(Cl)cc5F)n4)CC3)n(C)c2c1 ; CS(=O)(=O)OCC1(F)CC1 ; COC(=O)c1cc(OCC2(F)CC2)c2nc(CN3CCC(c4cccc(OCc5ccc(Cl)cc5F)n4)CC3)n(C)c2c1 ; Cn1c(CN2CCC(c3cccc(OCc4ccc(Cl)cc4F)n3)CC2)nc2c(OCC3(F)CC3)cc(C(=O)O)cc21 ; OCC1(F)CC1 ; CS(=O)(=O)OCC1(F)CC1]

### General Procedure for Preparation of Intermediate 1B:

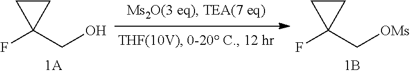 [SMILES: OCC1(F)CC1 ; CS(=O)(=O)OCC1(F)CC1]

To a solution of 1A (200 mg, 2.22 mmol, 1 eq) in THF (2 mL) was added TEA (1.57 g, 15.54 mmol, 2.16 mL, 7 eq) at 20° C. The mixture was degassed and purged with N₂ for 3 times, and then cooled to 0° C. Then Ms₂O (1.16 g, 6.66 mmol, 3 eq) in THF (0.5 mL) was added dropwise, keeping the internal temperature below 10° C. The mixture was stirred at 20° C. for 12 hr. HNMR showed 1A was consumed completely and desired was detected. The reaction mixture was added to 5 mL H₂O, IN FA was added to the mixture to adjust pH-6-7, then the mixture was extracted with DCM (10 mL×3), the combined organic phase was washed with brine 5 mL, dried over anhydrous Na₂SO₄, filtered and concentrated under reduced pressure to give a residue. 1B (330 mg, 1.96 mmol, 88.39% yield) was obtained as a yellow oil.

¹H NMR (400 MHz, CHCl3-d) δ=4.48 (d, J=22 Hz, 2H), 3.11 (s, 3H), 1.30-1.19 (m, 2H), 0.91-0.83 (m, 2H)

### General Procedure for Preparation of Intermediate 2:

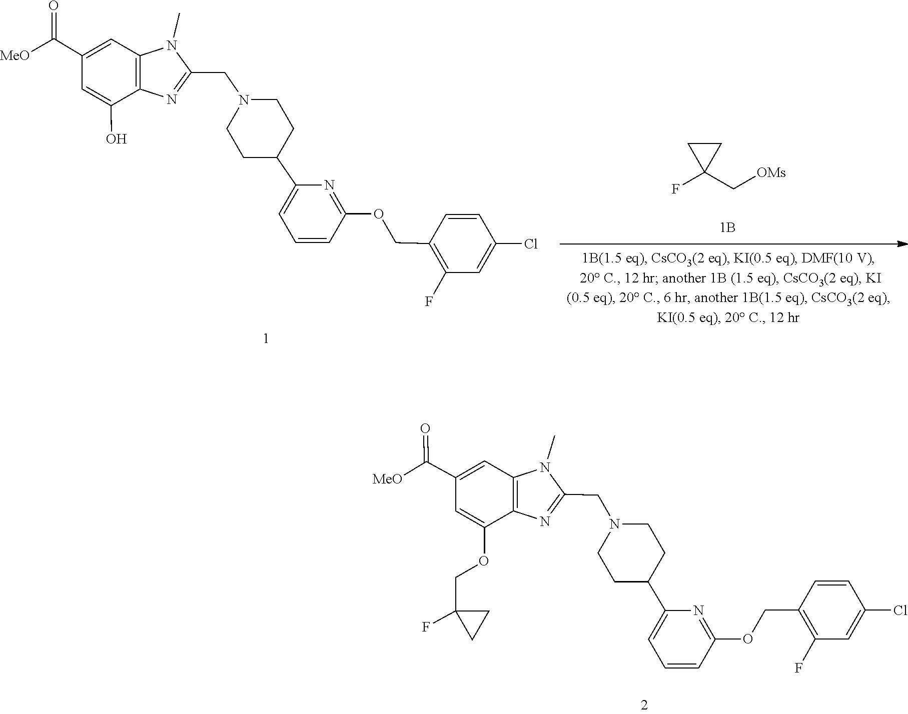 [SMILES: COC(=O)c1cc(O)c2nc(CN3CCC(c4cccc(OCc5ccc(Cl)cc5F)n4)CC3)n(C)c2c1 ; CS(=O)(=O)OCC1(F)CC1 ; COC(=O)c1cc(OCC2(F)CC2)c2nc(CN3CCC(c4cccc(OCc5ccc(Cl)cc5F)n4)CC3)n(C)c2c1]

To a solution of 1 (80 mg, 148.42 μmol, 1 eq) in DMF (0.8 mL) was added 1B (37.44 mg, 222.64 μmol, 1.5 eq, Cs₂CO₃ (96.72 mg, 296.85 μmol, 2 eq) and KI (12.32 mg, 74.21 μmol, 0.5 eq) at 20° C. The mixture was stirred at 20° C. for 12 hr. Then another 1B (37.44 mg, 222.64 μmol, 1.5 eq), KI (12.32 mg, 74.21 μmol, 0.5 eq) and Cs₂CO₃ (96.72 mg, 296.85 μmol, 2 eq) was added to the mixture and stirred at 20° C. for 6 hr. Then another 1B (37.44 mg, 222.64 μmol, 1.5 eq), KI (12.32 mg, 74.21 μmol, 0.5 eq) and Cs₂CO₃ (96.72 mg, 296.85 μmol, 2 eq) was added to the mixture and stirred at 20° C. for 12 hr. LCMS (RT=1.712 min) showed 1 was consumed completely and one main peak with desired m/s was detected. The reaction mixture was added to 10 mL H₂O and extracted with EtOAc (10 mL×3), the combined organic phase was washed with brine 10 mL, dried over Na₂SO₄, filtered, and concentrated under reduced pressure to give a residue. The residue was purified by column chromatography (SiO₂, Petroleum ether/Ethyl acetate=1/0 to 0/1). Then the crude product was triturated with PE (5 mL) at 20° C. for 10 min. The mixture was filtered and collected. It was repeated for 5 times. The final product was dried under vacuum. 2 (50 mg, 76.10 μmol, 51.27% yield, 93% purity) was obtained as a white solid.

LCMS: RT=1.714 min, MS cal.: 610.2, [M+H]⁺=611.3

### General Procedure for Preparation of Compound 188 (3):

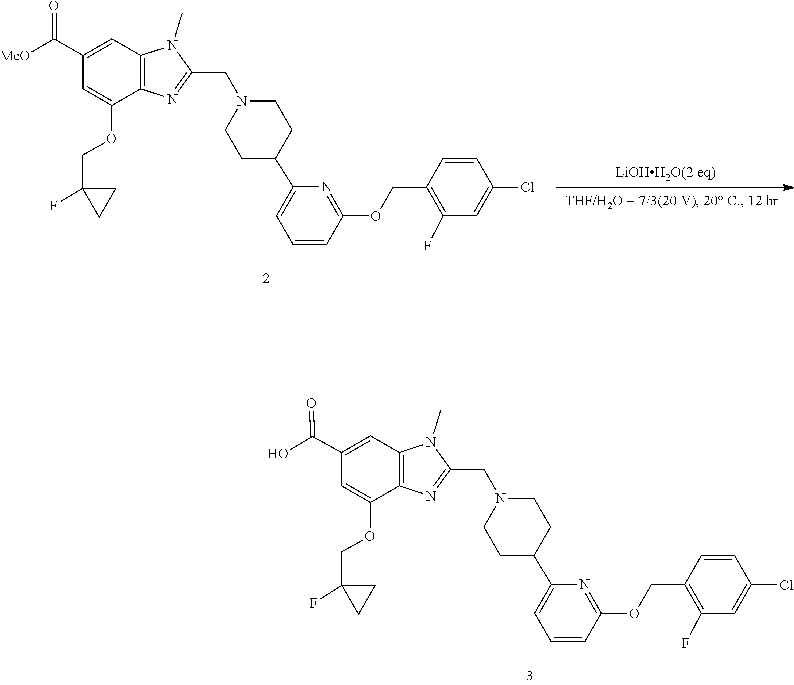 [SMILES: COC(=O)c1cc(OCC2(F)CC2)c2nc(CN3CCC(c4cccc(OCc5ccc(Cl)cc5F)n4)CC3)n(C)c2c1 ; Cn1c(CN2CCC(c3cccc(OCc4ccc(Cl)cc4F)n3)CC2)nc2c(OCC3(F)CC3)cc(C(=O)O)cc21]

To a solution of 2 (40 mg, 65.46 μmol, 1 eq) in THF (0.8 mL) was added LiOH·H₂O (5.49 mg, 130.92 μmol, 2 eq) in H₂O (0.3 mL). The mixture was stirred at 20° C. for 12 hr. Then LiOH·H₂O (2.75 mg, 65.46 μmol, 1 eq) was added to the mixture and stirred at 20° C. for 6 hr. LCMS (RT=1.197 min) showed 2 consumed completely and one main peak with desired m/s was detected. The reaction mixture was filtered. The residue was purified by prep-HPLC (column: Waters Xbridge Prep OBD C18 150*40 mm*10 um; mobile phase: [H₂O (10 mM NH₄HCO₃)-ACN]; gradient: 15%-65% B over 8.0 min). 2-((4-(6-((4-Chloro-2-fluorobenzyl)oxy)pyridin-2-yl)piperidin-1-yl)methyl)-4-((1-fluorocyclopropyl)methoxy)-1-methyl-1H-benzo[d]imidazole-6-carboxylic acid (9.72 mg, 15.53 μmol, 23.73% yield, 95.42% purity) was obtained as a white solid.

LCMS: RT=2.827 min, MS cal.: 596.2, [M+H]⁺=597.2

HPLC: RT=12.566 min, purity: 95.42%

¹H NMR (400 MHz, DMSO-d6) δ=7.80 (s, 1H), 7.62 (t, J=7.6 Hz, 1H), 7.55 (t, J=8.4 Hz, 1H), 7.45 (dd, J=2.0, 10.4 Hz, 1H), 7.31-7.25 (m, 2H), 6.87 (d, J=7.2 Hz, 1H), 6.66 (d, J=8.4 Hz, 1H), 5.36 (s, 2H), 4.55 (d, J=22.8 Hz, 2H), 3.92 (s, 3H), 3.82 (s, 2H), 2.93 (br d, J=10.8 Hz, 2H), 2.63-2.55 (m, 1H), 2.20 (br t, J=11.2 Hz, 2H), 1.85-1.65 (m, 4H), 1.24-1.09 (m, 2H), 0.92 (q, J=7.6 Hz, 2H)

### Example A117—Preparation of Compound 190. 2-((4-(6-((4-Chloro-2-fluorobenzyl)oxy)pyridin-2-yl)piperidin-1-yl)methyl)-4-(difluoromethoxy)-7-fluoro-1-methyl-1H-benzo[d]imidazole-6-carboxylic acid (Compound 190) (11)

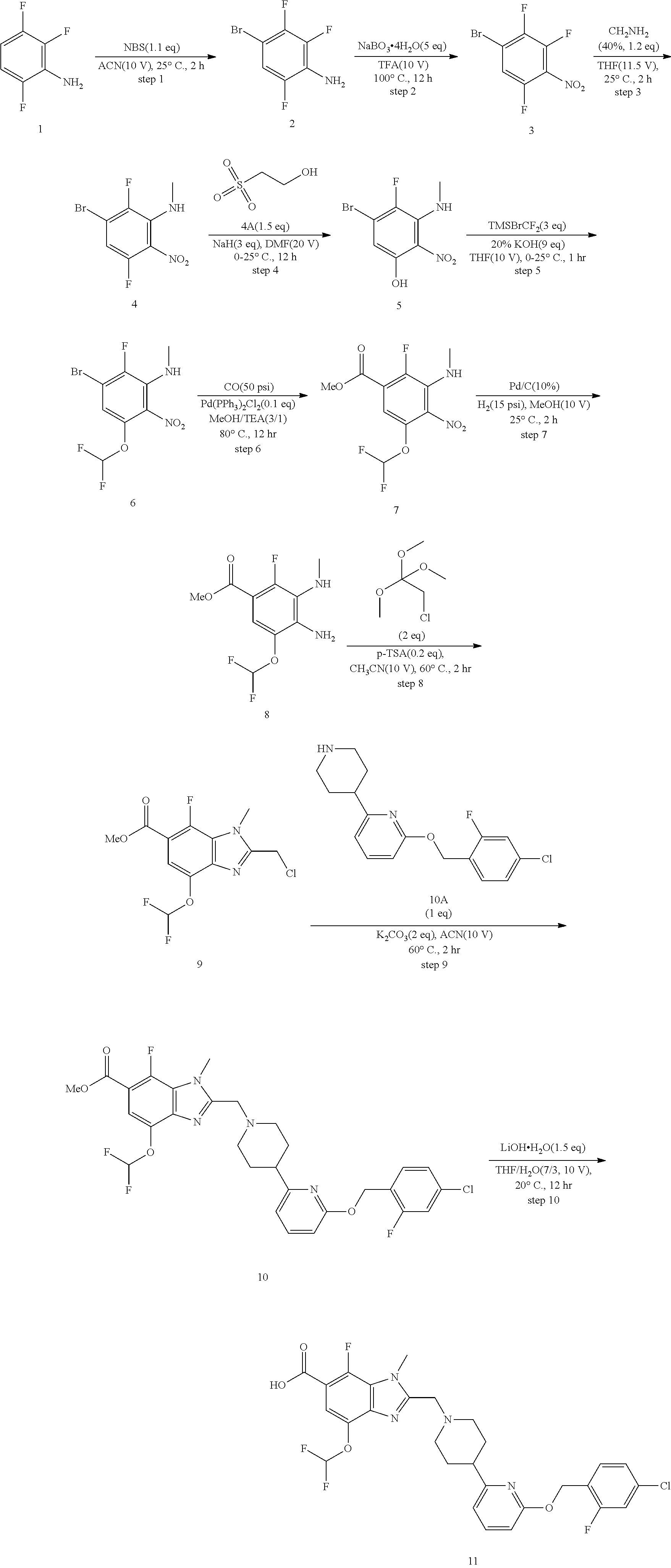

### General Procedure for Preparation of Intermediate 2:

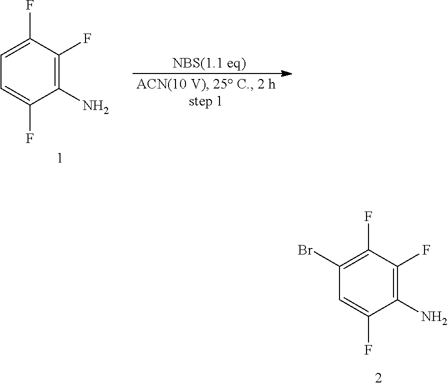 [SMILES: Nc1c(F)ccc(F)c1F ; Nc1c(F)cc(Br)c(F)c1F]

To a solution of 1 (5 g, 33.99 mmol, 1 eq) in ACN (50 mL) was added NBS (6.65 g, 37.39 mmol, 1.1 eq) at 0° C. The mixture was stirred at 25° C. for 2 hr. LCMS (Rt=1.147) showed 1 was consumed completely and desired mass was detected. The reaction mixture was added H₂O (100 mL) and extracted with DCM (40 mL*3). The combined organic layers were washed with brine (50 mL*2), dried over Na₂SO₄, filtered, and concentrated under reduced pressure to give a crude product. The residue was purified by column chromatography (SiO₂, Petroleum ether/Ethyl acetate=100/1 to 5/1). 2 (6.6 g, 29.20 mmol, 86% yield) was obtained as a brown oil.

LCMS: RT=1.147 min, MS cal.: 224.9, [M−H]⁻=223.8/225.8

1H NMR (400 MHz, CHCl3-d) δ=7.02 (m, 1H), 3.88 (br s, 2H)

### General Procedure for Preparation of Intermediate 3:

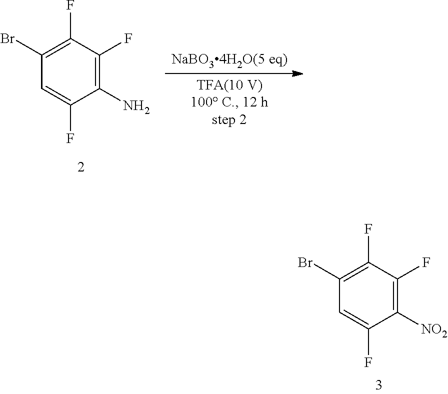 [SMILES: Nc1c(F)cc(Br)c(F)c1F ; O=[N+]([O-])c1c(F)cc(Br)c(F)c1F]

The reactions were carried out in parallels as two batches. Two reactions were combined for workup.

To a solution of 2 (2.8 g, 12.39 mmol, 1 eq) in TFA (28 mL) was added sodium; 3-oxidodioxaborirane; tetrahydrate (9.53 g, 61.95 mmol, 5 eq) at 20° C. The mixture was stirred at 100° C. for 12 hr. HPLC (Rt=2.345) showed 2 was consumed completely. The reaction mixture was concentrated under reduced pressure to remove TFA. The residue was diluted with H₂O (50 mL) and extracted with DCM (20 mL*3). The combined organic layers were washed with brine (50 mL*2), dried over Na₂SO₄, filtered, and concentrated under reduced pressure to give a residue. The residue was purified by column chromatography (SiO₂, Petroleum ether/Ethyl acetate=1/0 to 10/1). 3 (3.5 g, 13.67 mmol, 55% yield) was obtained as an orange oil.

HPLC: RT=2.345 min, purity: 81%

1H NMR (400 MHz, CHCl3-d) δ=7.43-7.38 (m, 1H)

### General Procedure for Preparation of Intermediate 4:

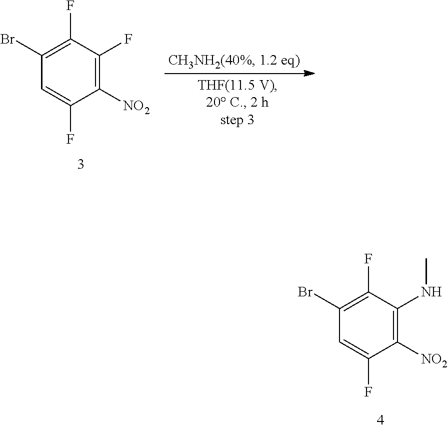

The reaction mixture was combined with the small scale (500 mg). Two reactions were combined for purification.

3 (3.3 g, 12.89 mmol, 1 eq) was charged to the three-necked round bottom flask, then THF (33 mL) was added at 20° C. under N₂. At 20° C. (inner temperature), methanamine (1.20 g, 15.47 mmol, 40% purity, 1.2 eq) in THF (5 mL) was added to the reaction mixture under N₂ atmosphere. After the addition, the mixture was degassed and purged with N₂ for 3 times, the mixture was stirred at 20° C. for 2 hr. LCMS (Rt=1.266) showed 3 was consumed completely and desired mass was detected. The reaction mixture was quenched by addition solution of 1 N FA to adjust pH=7, and then diluted with H₂O (100 mL) and extracted with EtOAc (40 mL*3). The combined organic layers were washed with brine (100 mL*2), dried over Na₂SO₄, filtered, and concentrated under reduced pressure to give a residue. The residue was purified by column chromatography (SiO₂, Petroleum ether/Ethyl acetate=1/0 to 5/1). 4 (2.8 g, 10.49 mmol, 70% yield) was obtained as an orange solid.

LCMS: RT=1.266 min, MS cal.: 266.0, [M−H]⁻=264.8, 266.8

1H NMR (400 MHz, CHCl3-d) δ=6.68 (dd, J=5.2, 10.0 Hz, 1H), 6.56 (br d, J, 1H), 3.17 (d, J=6.8 Hz, 3H)

### General Procedure for Preparation of Intermediate 5:

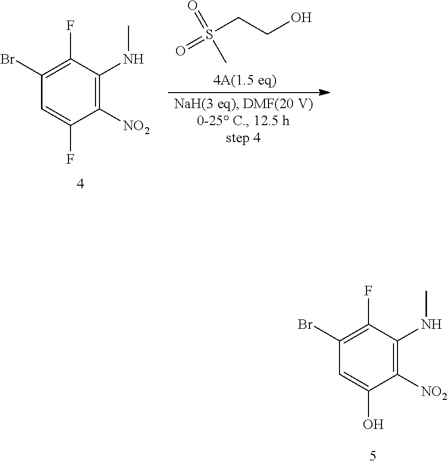

4 (1.2 g, 4.49 mmol, 1 eq) in DMF (12 mL) was charged to the three-necked round bottom flask, then 4A (836.94 mg, 6.74 mmol, 1.5 eq) was added at 25° C. under N₂. At 0° C. (inner temperature), NaH (539.22 mg, 13.48 mmol, 60% purity, 3 eq) was added to the reaction mixture under N₂ atmosphere and stirred for 0.5 h. Then the mixture was added DMF (12 mL) and stirred at 25° C. for 12 hr. LCMS (Rt=1.020) showed 4 was consumed completely and desired mass was detected. The reaction mixture was quenched by addition ammonium chloride solution at 0° C., and then diluted with H₂O (100 mL) and extracted with EtOAc (40 mL*3). The combined organic layers were washed with brine (55 mL*2), dried over Na₂SO₄, filtered and concentrated under reduced pressure to give a residue. The residue was purified by column chromatography (SiO₂, Petroleum ether/Ethyl acetate=99/1 to 8/1). 5 (600 mg, 2.26 mmol, 50% yield) was obtained as a dark red solid.

LCMS: RT=1.020 min, MS cal.: 264.0, [M−H]⁻=262.9, 264.9

1H NMR (400 MHz, CHCl3-d) δ=11.30 (s, 1H), 8.52-8.25 (br s, 1H), 6.49 (d, J=5.6 Hz, 1H), 3.29 (d, J=8.4 Hz, 3H)

### General Procedure for Preparation of Intermediate 6:

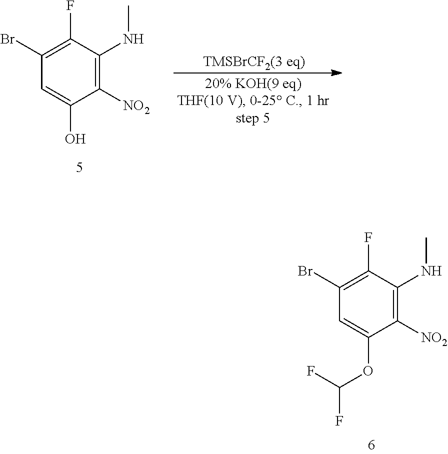 [SMILES: CNc1c(F)c(Br)cc(O)c1[N+](=O)[O-] ; CNc1c(F)c(Br)cc(OC(F)F)c1[N+](=O)[O-]]

THF (4.5 mL) was charged to the three-necked round bottom flask, then 5 (500 mg, 1.89 mmol, 1 eq) and KOH (4.76 g, 16.98 mmol, 20% purity, 9 eq) was added to the mixture at 0° C. for 10 min. At 0° C., [bromo (difluoro) methyl]-trimethyl-silane (1.15 g, 5.66 mmol, 3 eq) in THF (0.5 mL) was added dropwise to the reaction mixture. After the addition, the mixture was stirred at 25° C. for 50 min. LCMS (Rt=1.330) showed 5 was consumed completely and desired mass was detected. The reaction mixture was diluted with H₂O (30 mL) and extracted with EtOAc (10 mL*3). The combined organic layers were washed with brine (10 mL*2), dried over Na₂SO₄, filtered, and concentrated under reduced pressure to give a crude product. 6 (530 mg, 1.68 mmol, 89% yield) was obtained as an orange solid.

LCMS: RT=1.330 min, MS cal.: 314.0, [M+H]⁺=312.9, 314.9

1H NMR (400 MHz, CHCl3-d) δ=6.80 (d, J=5.2 Hz, 1H), 6.49 (t, J=72.8 Hz, 1H), 5.50 (br s, 1H), 3.09 (d, J=4.8 Hz, 3H)

### General Procedure for Preparation of Intermediate 7:

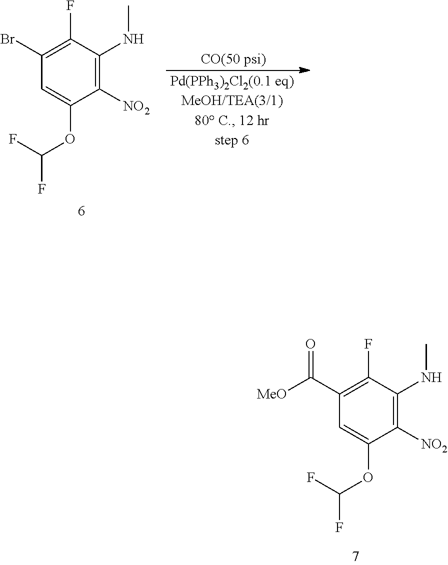 [SMILES: CNc1c(F)c(Br)cc(OC(F)F)c1[N+](=O)[O-] ; CNc1c(F)c(C(=O)OC)cc(OC(F)F)c1[N+](=O)[O-]]

TEA (2 mL) was charged to 75 mL hydrogenated bottle, then 6 (530 mg, 1.68 mmol, 1 eq) in MeOH (6 mL) and Pd(PPh₃)₂Cl₂ (118.08 mg, 168.23 μmol, 0.1 eq) was added to the mixture at 20° C. After the addition, the mixture was degassed and purged with CO (50 Psi) for 3 times. The mixture was stirred at 80° C. for 12 hr. LCMS (Rt=1.231) showed 6 was consumed completely and desired mass was detected. The reaction mixture was filtered, and the filter cake was washed with EtOAc (3 mL). The filtrate was diluted with H₂O (15 mL) and extracted with EtOAc (5 mL*3). The combined organic layers were washed with brine (10 mL*2), dried over Na₂SO₄, filtered, and concentrated under reduced pressure to give a residue. The residue was purified by column chromatography (SiO₂, Petroleum ether/Ethyl acetate=99/1 to 3/1). 7 (120 mg, 407.91 μmol, 24% yield) was obtained as a yellow solid.

LCMS: RT=1.231 min, MS cal.: 294.0, [M−H]⁻=293.0

1H NMR (400 MHz, CHCl3-d) δ=7.04 (d, J=5.2 Hz, 1H), 6.51 (t, J=72.8 Hz, 1H), 5.11 (br s, 1H), 3.96 (s, 3H), 3.05 (d, J=2.8 Hz, 3H)

### General Procedure for Preparation of Intermediate 8:

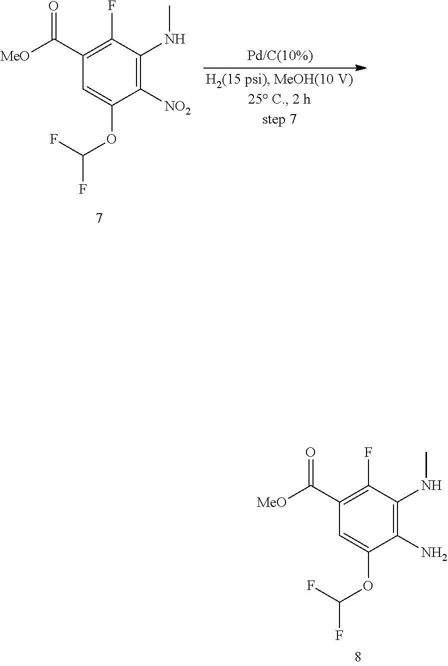 [SMILES: CNc1c(F)c(C(=O)OC)cc(OC(F)F)c1[N+](=O)[O-] ; CNc1c(N)c(OC(F)F)cc(C(=O)OC)c1F]

MeOH (1.2 mL) was charged to the round bottom flask, then Pd/C (40 mg) was added at 20° C. under Ar. At 20° C. (inner temperature), 7 (120 mg, 407.91 μmol, 1 eq) was added to the reaction mixture under N₂ atmosphere. After the addition, the mixture was degassed and purged with H₂ for 3 times, the mixture was stirred at 20° C. for 2 hr under H₂ (15 psi). LCMS (Rt=0.395) showed 7 was consumed completely and desired mass was detected. The reaction mixture was diluted with MeOH (3 ml) and filtered through celite pad. The filter cake was rinsed with MeOH (5 ml*3) and the filtrate was concentrated under reduced pressure to give a crude product. The residue was purified by column chromatography (SiO₂, Petroleum ether/Ethyl acetate=10/1 to 3/1). 8 (80 mg, 302.80 μmol, 74% yield) was obtained as a white solid.

LCMS: RT=0.395 min, MS cal.: 264.1, [M+H]⁺=265.1

1H NMR (400 MHz, DMSO-d6) δ=7.10 (d, J=6.4 Hz, 1H), 7.03 (t, J=72.8, 1H), 5.73 (s, 2H), 4.33 (m, 1H), 3.75 (s, 3H), 2.73 (dd, J=2.8, 5.2 Hz, 3H)

### General Procedure for Preparation of Intermediate 9:

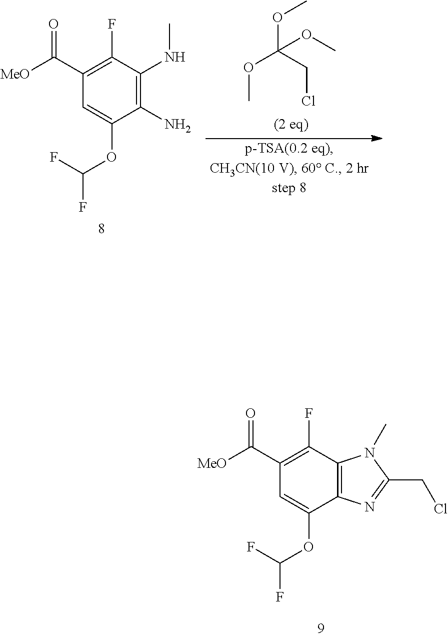

A solution of 8 (80 mg, 302.80 μmol, 1 eq) and 2-chloro-1,1,1-trimethoxy-ethane (93.62 mg, 605.60 μmol, 81.62 μL, 2 eq) in ACN (1 mL) was added p-TSA (10.43 mg, 60.56 μmol, 0.2 eq) at 20° C. The mixture was stirred at 60° C. for 2 hr. LCMS (Rt=0.478) showed 8 was consumed completely and desired mass was detected. The reaction mixture was diluted with H₂O (3 mL) and extracted with EtOAc (1 mL*3). The combined organic layers were washed with brine (2 mL*2), dried over Na₂SO₄, filtered, and concentrated under reduced pressure to give a residue. 9 (80 mg, 247.93 μmol, 81.88% yield) was obtained as a yellow oil.

LCMS: RT=0.478 min, MS cal.: 322.0, [M+H]⁺=323.1

1H NMR (400 MHz, CHCl3-d) δ=7.59 (d, J=5.2 Hz, 1H), 7.12 (t, J=74.0, 1H), 4.84 (s, 2H), 4.13 (s, 3H), 3.98 (s, 3H)

### General Procedure for Preparation of Intermediate 9:

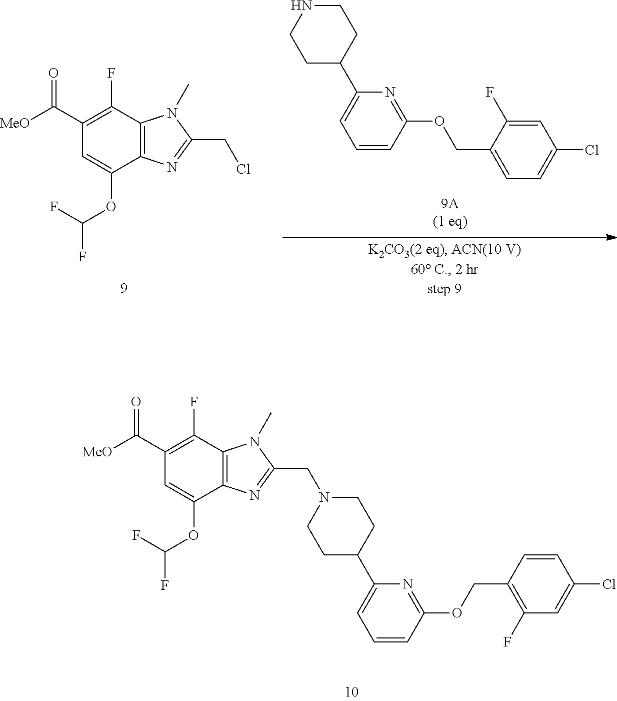 [SMILES: COC(=O)c1cc(OC(F)F)c2nc(CCl)n(C)c2c1F ; Fc1cc(Cl)ccc1COc1cccc(C2CCNCC2)n1 ; COC(=O)c1cc(OC(F)F)c2nc(CN3CCC(c4cccc(OCc5ccc(Cl)cc5F)n4)CC3)n(C)c2c1F]

A mixture of 9 (60 mg, 185.95 μmol, 1 eq), 9A (59.65 mg, 185.95 μmol, 1 eq), K₂CO₃ (51.40 mg, 371.90 μmol, 2 eq) in ACN (1 mL) was degassed and purged with N₂ for 3 times at 20° C., and then the mixture was stirred at 60° C. for 2 hr under N₂ atmosphere. LCMS (Rt=0.515) showed 9 was consumed completely and desired mass was detected. The reaction mixture was diluted with H₂O (3 mL) and extracted with DCM (1 mL*3). The combined organic layers were washed with brine (2 mL*2), dried over Na₂SO₄, filtered, and concentrated under reduced pressure to give a residue. The residue was purified by column chromatography (SiO₂, Petroleum ether/Ethyl acetate=10/1 to 1/1). 10 (60 mg, 98.85 μmol, 53% yield) was obtained as a white solid.

LCMS: RT=0.515 min, MS cal.: 606.2, [M−H]⁻=607.3

### General Procedure for Preparation of Compound 190 (11):

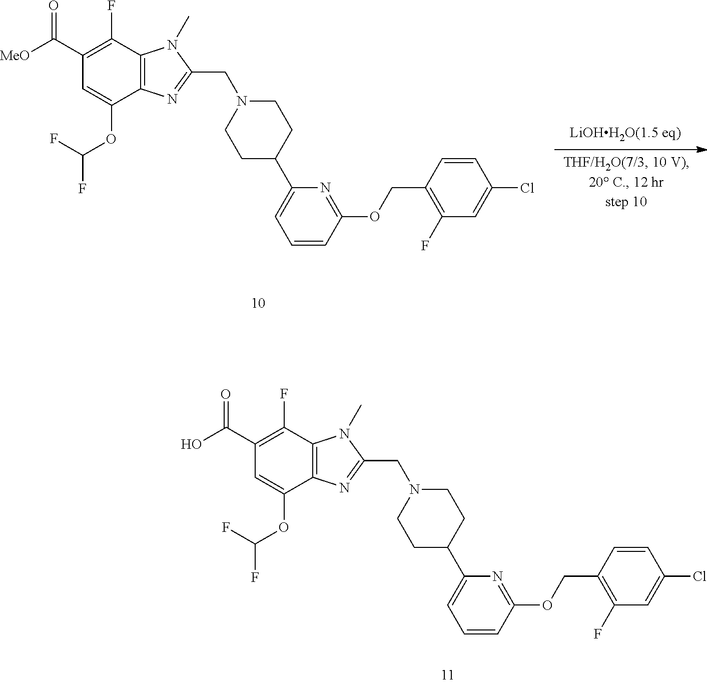 [SMILES: COC(=O)c1cc(OC(F)F)c2nc(CN3CCC(c4cccc(OCc5ccc(Cl)cc5F)n4)CC3)n(C)c2c1F ; Cn1c(CN2CCC(c3cccc(OCc4ccc(Cl)cc4F)n3)CC2)nc2c(OC(F)F)cc(C(=O)O)c(F)c21]

A mixture of 10 (50 mg, 82.37 μmol, 1 eq), LiOH·H₂O (5.19 mg, 123.56 μmol, 1.5 eq) in THF (0.35 mL) and H₂O (0.15 mL) was degassed and purged with N₂ for 3 times, and then the mixture was stirred at 20° C. for 12 hr under N₂ atmosphere. LCMS (Rt=1.226) showed 10 was consumed completely and desired mass was detected. The reaction mixture was filtered and concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 30%-70% B over 8.0 min). 2-((4-(6-((4-Chloro-2-fluorobenzyl)oxy)pyridin-2-yl)piperidin-1-yl)methyl)-4-(difluoromethoxy)-7-fluoro-1-methyl-1H-benzo[d]imidazole-6-carboxylic acid (28.39 mg, 47.88 μmol, 58% yield) was obtained as a white solid.

LCMS: RT=1.226 min, MS cal.: 592.2, [M+H]⁺=593.0

HPLC: RT=9.452 purity: 99.73%

¹H NMR (400 MHz, MeOH-d4) δ=7.66 (s, 1H), 7.65-7.60 (m, 1H), 7.56 (t, J=8.4 Hz, 1H), 7.47 (t, J=74.4, 1H), 7.44 (dd, J=2.0, 10.4 Hz, 1H), 7.34 (d, J=4.8 Hz, 1H), 7.31-7.26 (m, 1H), 6.87 (d, J=7.2 Hz, 1H), 6.67 (d, J=8.1 Hz, 1H), 5.36 (s, 2H), 4.07 (s, 3H), 3.83 (s, 2H), 2.93 (d, J=11.2 Hz, 2H), 2.65-2.55 (m, 1H), 2.27-2.15 (m, 2H), 1.84-1.63 (m, 4H)

### Example A118—Preparation of Compound 191. (S)-2-((4-(6-((4-chloro-2-fluorobenzyl)oxy)pyridin-2-yl)piperidin-1-yl)methyl)-4-(difluoromethoxy)-1-(oxetan-2-ylmethyl)-1H-benzo[d]imidazole-5-carboxylic acid (Compound 191) (6)

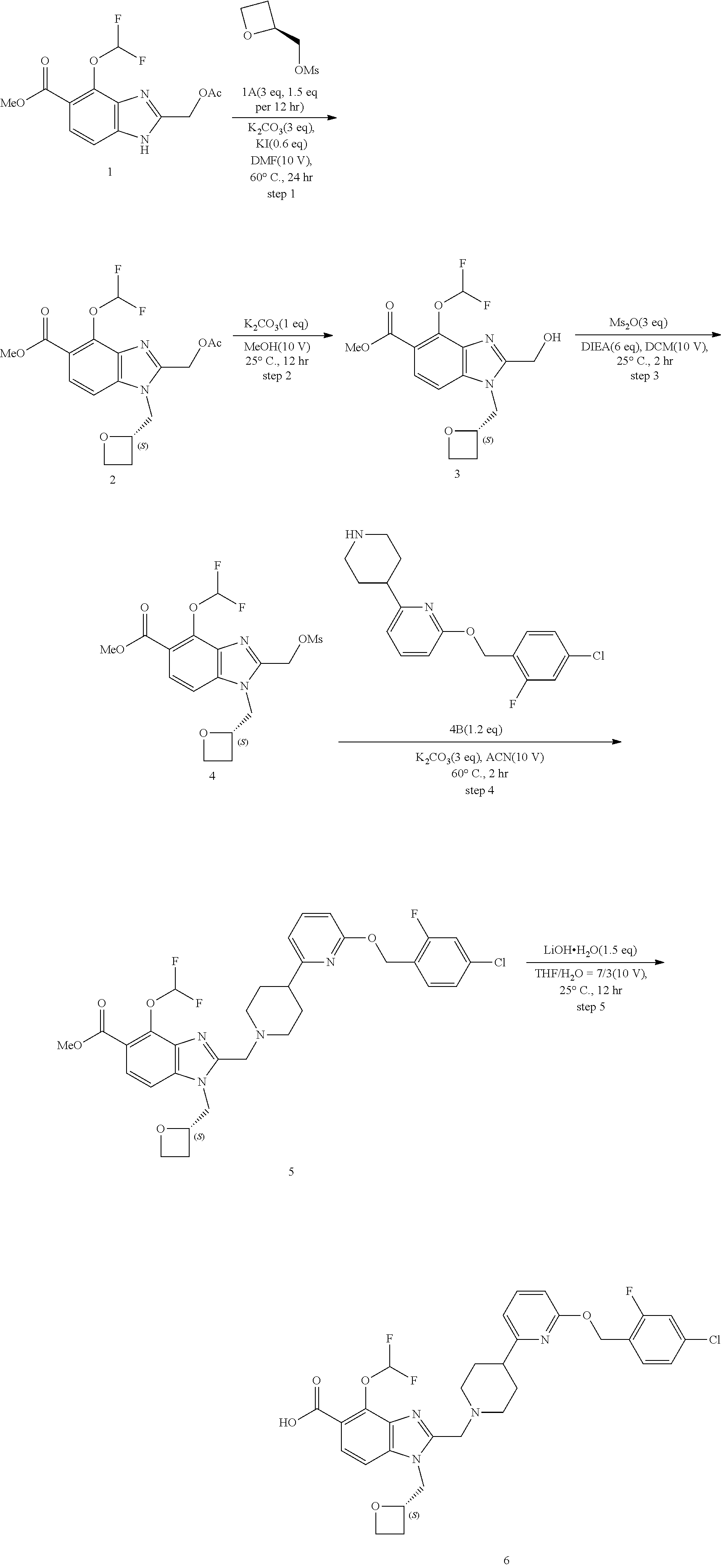

### General Procedure for Preparation of Intermediate 2:

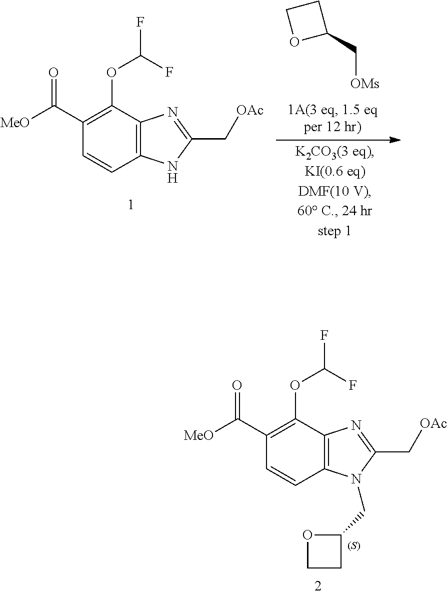

A mixture of 1 (120 mg, 381.87 μmol, 1 eq), 1A (95.20 mg, 572.81 μmol, 1.5 eq), K₂CO₃ (158.34 mg, 1.15 mmol, 3 eq), KI (38.03 mg, 229.12 μmol, 0.6 eq) in DMF (1 mL) was degassed and purged with N₂ for 3 times, and then the mixture was stirred at 60° C. for 12 hr under N₂ atmosphere. Then the mixture was added 1A (95.20 mg, 572.81 μmol, 1.5 eq), the mixture was stirred at 60° C. for 12 hr under N₂ atmosphere. LCMS (RT=1.051 min) showed 1 was consumed completely and one main peak with desired mass was detected. The reaction mixture was diluted with H₂O 5 mL and extracted with DCM (5 mL*4). The combined organic layers were dried over Na₂SO₄, filtered, and concentrated under reduced pressure to give a residue. The residue was purified by column chromatography (SiO₂, Petroleum ether/Ethyl acetate=100/1 to 0/1). 2 (130 mg, 338.25 μmol, 88% yield) was obtained as a yellow solid.

### General Procedure for Preparation of Intermediate 3:

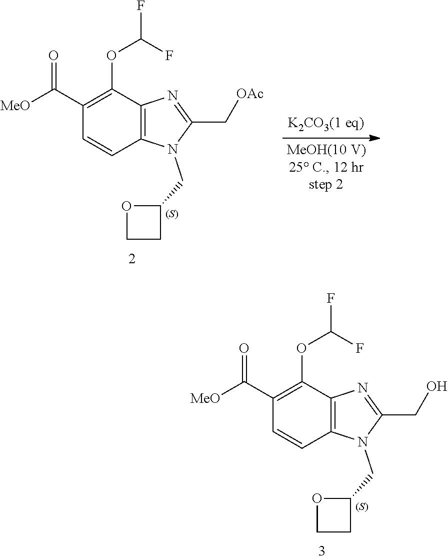

A mixture of 2 (120 mg, 312.23 μmol, 1 eq), K₂CO₃ (43.15 mg, 312.23 μmol, 1 eq) in MeOH (1.2 mL) was degassed and purged with N₂ for 3 times, and then the mixture was stirred at 25° C. for 12 hr under N₂ atmosphere. LCMS (RT=0.910 min) showed 2 was consumed completely and one main peak with desired mass was detected. The reaction mixture was filtered and concentrated under reduced pressure to give a residue. The residue was purified by prep-HPLC (column: Waters Xbridge Prep OBD C₁₈ 150*40 mm*10 um; mobile phase: [H₂O (10 mM NH₄HCO₃)-ACN]; gradient: 5%-35% B over 8.0 min). 3 (20 mg, 58.43 μmol, 19% yield) was obtained as a white solid.

LCMS: RT=0.910 min, MS cal.: 342.1, [M+H]⁺=342.9

¹H NMR (400 MHz, DMSO-d₆) δ=7.73-7.70 (m, 1H), 7.71 (t, J=74.8 Hz, 1H), 7.67-7.64 (m, 1H), 5.73 (s, 1H), 5.06 (dq, J=2.8, 7.2 Hz, 1H), 4.85-4.68 (m, 3H), 4.62-4.54 (m, 1H), 4.51-4.44 (m, 1H), 4.38 (td, J=6.0, 8.8 Hz, 1H), 3.85 (s, 3H), 2.74-2.64 (m, 1H), 2.45-2.37 (m, 1H)

### General Procedure for Preparation of Intermediate 4:

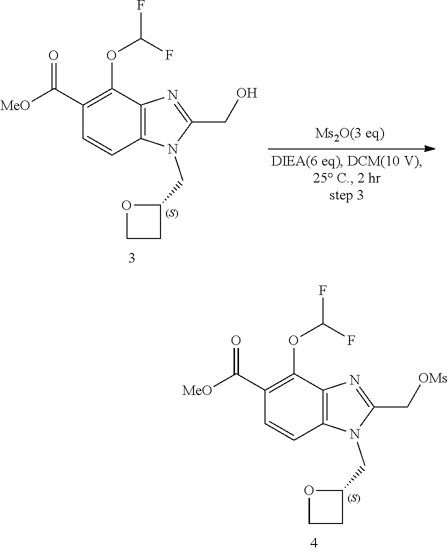

A mixture of 3 (20 mg, 58.43 μmol, 1 eq), Ms₂O (30.53 mg, 175.29 μmol, 3 eq), DIEA (45.31 mg, 350.58 μmol, 61.06 μL, 6 eq) in DCM (0.5 mL) was degassed and purged with N₂ for 3 times, and then the mixture was stirred at 25° C. for 2 hr under N₂ atmosphere. LCMS (RT=1.056 min) showed 3 was consumed completely and one main peak with desired mass was detected. The reaction mixture was added HCOOH at 25° C. until pH=7-8, and then diluted with H₂O 5 mL and extracted with DCM 5 mL*4. The combined organic layers were dried over Na₂SO₄, filtered, and concentrated under reduced pressure to give a residue. 4 (24 mg, crude) was used into the next step without further purification.

### General Procedure for Preparation of Intermediate 5:

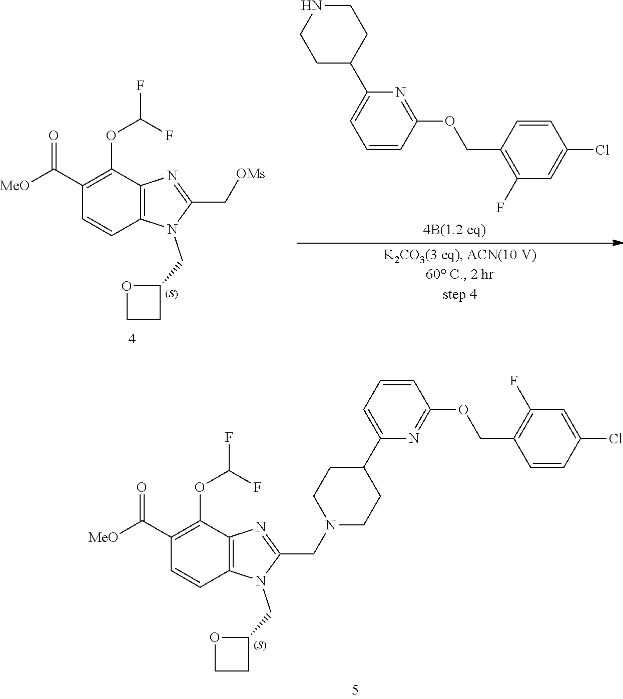

A mixture of 4 (24 mg, 57.09 μmol, 1 eq), 4B (21.98 mg, 68.51 μmol, 1.2 eq), K₂CO₃ (23.67 mg, 171.27 μmol, 3 eq) in ACN (0.5 mL) was degassed and purged with N₂ for 3 times, and then the mixture was stirred at 60° C. for 2 hr under N₂ atmosphere. LCMS (RT=1.732 min) showed 4 was consumed completely and one main peak with desired mass was detected. The reaction mixture was diluted with H₂O 5 mL and extracted with DCM (5 mL*4). The combined organic layers were dried over Na₂SO₄, filtered and concentrated under reduced pressure to give a residue. The residue was purified by prep-TLC (SiO₂, Petroleum ether/Ethyl acetate=0/1). 5 (27 mg, 41.86 μmol, 73% yield) was obtained as a yellow oil.

LCMS: RT=1.736 min, MS cal.: 644.2, [M+H]⁺=645.1

¹H NMR (400 MHz, DMSO-d₆) δ=7.73-7.70 (m, 1H), 7.69 (t, J=76 Hz, 1H), 7.67-7.61 (m, 2H), 7.56 (t, J=8.0 Hz, 1H), 7.46 (dd, J=2.0, 10.0 Hz, 1H), 7.29 (dd, J=2.0, 8.0 Hz, 1H), 6.88 (d, J=7.2 Hz, 1H), 6.68 (d, J=8.0 Hz, 1H), 5.38 (s, 2H), 5.16-5.09 (m, 1H), 4.85-4.76 (m, 1H), 4.70-4.62 (m, 1H), 4.52-4.38 (m, 2H), 3.96 (d, J=13.6 Hz, 1H), 3.85 (s, 3H), 3.80 (d, J=13.6 Hz, 1H), 3.00 (d, J=12.0 Hz, 1H), 2.90-2.83 (m, 1H), 2.76-2.67 (m, 1H), 2.32-2.15 (m, 2H), 1.85-1.65 (m, 4H)

### General Procedure for Preparation of Compound 191 (6):

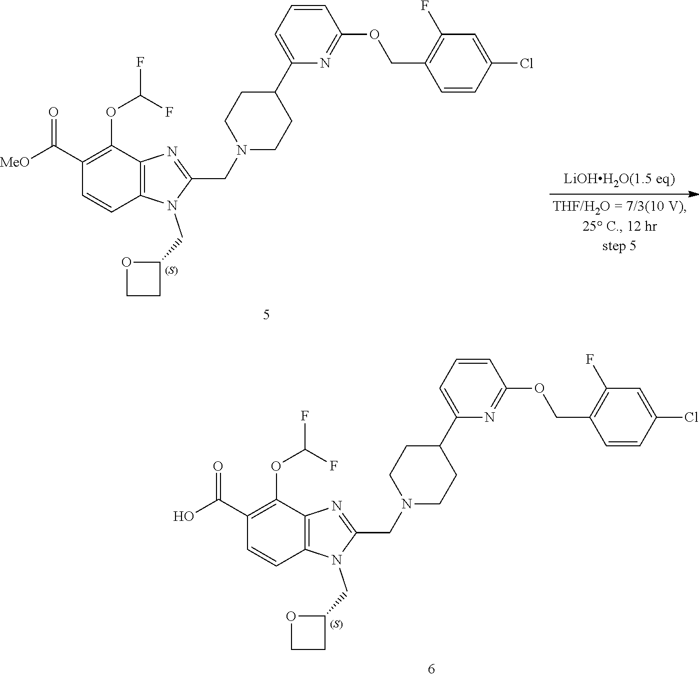

A mixture of 5 (17 mg, 26.35 μmol, 1 eq), LiOH·H₂O (1.66 mg, 39.53 μmol, 1.5 eq) in THF (0.35 mL) and H₂O (0.15 mL) was degassed and purged with N₂ for 3 times, and then the mixture was stirred at 25° C. for 12 hr under N₂ atmosphere. LCMS (RT=1.233 min) showed 5 was consumed completely and one main peak with desired mass was detected. The reaction mixture was filtered. The filtrate was purified by prep-HPLC (column: Waters Xbridge BEH C₁₈ 100*30 mm*10 um; mobile phase: [H₂O (10 mM NH₄HCO₃)-ACN]; gradient: 30%-65% B over 8.0 min). (S)-2-((4-(6-((4-chloro-2-fluorobenzyl)oxy)pyridin-2-yl)piperidin-1-yl)methyl)-4-(difluoromethoxy)-1-(oxetan-2-ylmethyl)-1H-benzo[d]imidazole-5-carboxylic acid (16.12 mg, 25.55 μmol) was obtained as a white solid.

LCMS: RT=2.463 min, MS cal.: 630.2, [M+H]⁺=631.2

HPLC: RT=12.321 min, purity: 98.17%

¹H NMR (400 MHz, DMSO-d₆) δ=7.70 (d, J=8.4 Hz, 1H), 7.67 (t, J=76 Hz, 1H), 7.66-7.59 (m, 2H), 7.56 (t, J=8.0 Hz, 1H), 7.49-7.43 (m, 1H), 7.27 (s, 1H), 6.88 (d, J=7.2 Hz, 1H), 6.68 (d, J=8.0 Hz, 1H), 5.42-5.34 (m, 2H), 5.12 (dd, J=3.2, 7.2 Hz, 1H), 4.83-4.75 (m, 1H), 4.69-4.61 (m, 1H), 4.51-4.38 (m, 2H), 3.95 (d, J=13.6 Hz, 1H), 3.79 (d, J=13.6 Hz, 1H), 3.00 (d, J=11.2 Hz, 1H), 2.86 (d, J=9.6 Hz, 1H), 2.75-2.66 (m, 1H), 2.65-2.58 (m, 1H), 2.45-2.39 (m, 1H), 2.33-2.14 (m, 2H), 1.84-1.65 (m, 4H)

### Example A119—Preparation of Compound 192. 2-((4-(6-((4-Chloro-2-fluorobenzyl)oxy)pyridin-2-yl)piperidin-1-yl)methyl)-1-(fluoromethyl)-7-methoxy-1H-benzo[d]imidazole-5-carboxylic acid (Compound 192) (5)

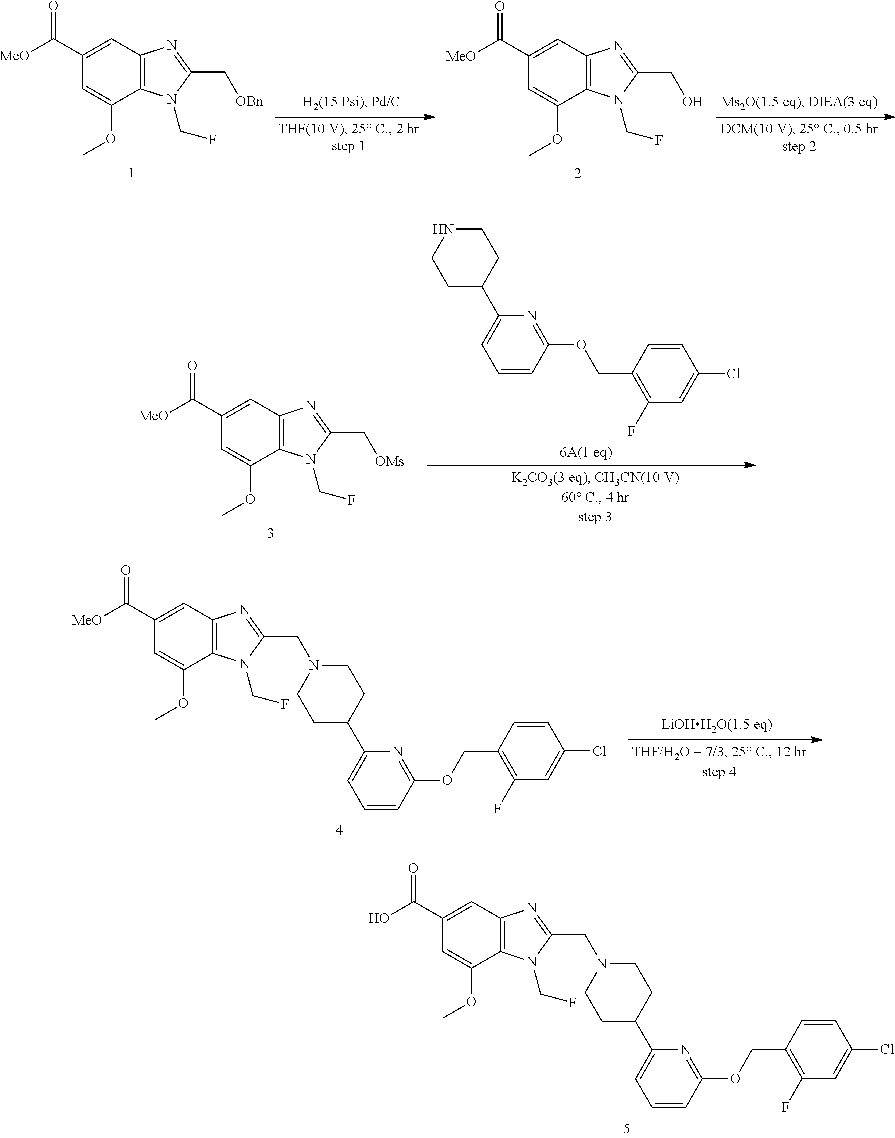 [SMILES: COC(=O)c1cc(OC)c2c(c1)nc(COCc1ccccc1)n2CF ; COC(=O)c1cc(OC)c2c(c1)nc(CO)n2CF ; COC(=O)c1cc(OC)c2c(c1)nc(COS(C)(=O)=O)n2CF ; Fc1cc(Cl)ccc1COc1cccc(C2CCNCC2)n1 ; COC(=O)c1cc(OC)c2c(c1)nc(CN1CCC(c3cccc(OCc4ccc(Cl)cc4F)n3)CC1)n2CF ; COc1cc(C(=O)O)cc2nc(CN3CCC(c4cccc(OCc5ccc(Cl)cc5F)n4)CC3)n(CF)c12]

### General Procedure for Preparation of Intermediate 2:

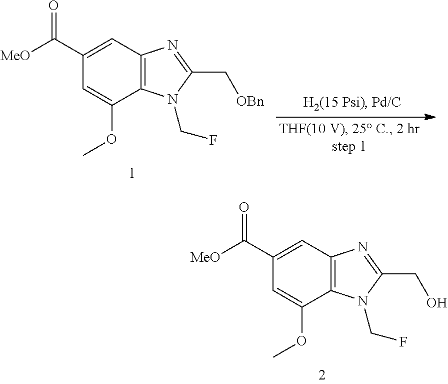 [SMILES: COC(=O)c1cc(OC)c2c(c1)nc(COCc1ccccc1)n2CF ; COC(=O)c1cc(OC)c2c(c1)nc(CO)n2CF]

THF (1 mL) was charged to the round bottom flask, then Pd/C (50 mg, 5% purity) was added at 25° C. under N₂. At 25° C., 1 (200 mg, 558.09 μmol, 1 eq) in THF (1 mL) was added to the reaction mixture at 25° C. under N₂ atmosphere. After the addition, the suspension was degassed and purged with H₂ for 3 times, and then the mixture was stirred under H₂ (15 Psi) atmosphere at 50° C. for 2 hr. LCMS showed 1 was consumed completely and one main peak with desired mass was detected. The suspension was filtered through a pad of celite, and filter cake was washed with THF (5 mL×3). The combined filtrates were concentrated to dryness to give a residue. 2 (140 mg, 521.92 μmol, 93.52% yield) was obtained as a white solid.

LCMS: RT=0.346 min, MS cal.: 268.1, [M+H]⁺=269.2

### General Procedure for Preparation of Intermediate 3:

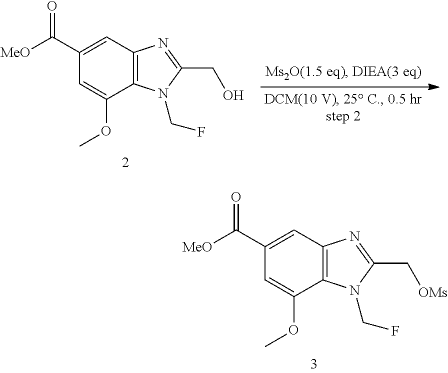 [SMILES: COC(=O)c1cc(OC)c2c(c1)nc(CO)n2CF ; COC(=O)c1cc(OC)c2c(c1)nc(COS(C)(=O)=O)n2CF]

To a solution of 2 (140 mg, 521.92 μmol, 1 eq) in DCM (2 mL) was added Ms₂O (136.37 mg, 782.88 μmol, 1.5 eq) and DIEA (202.36 mg, 1.57 mmol, 272.73 μL, 3 eq). The mixture was stirred at 25° C. for 0.5 hr. LCMS showed 2 was consumed completely and one main peak with desired mass was detected. The reaction mixture was diluted with H₂O (5 mL) and extracted with DCM (5 mL*3). The combined organic layers were washed with brine (3 mL*3), dried over Na₂SO₄, filtered and concentrated under reduced pressure to give a residue. 3 (180 mg, 519.73 μmol, 99.58% yield) was obtained as a yellow solid.

LCMS: RT=0.401 min, MS cal.: 346.1, [M+H]⁺=347.0

### General Procedure for Preparation of Intermediate 4:

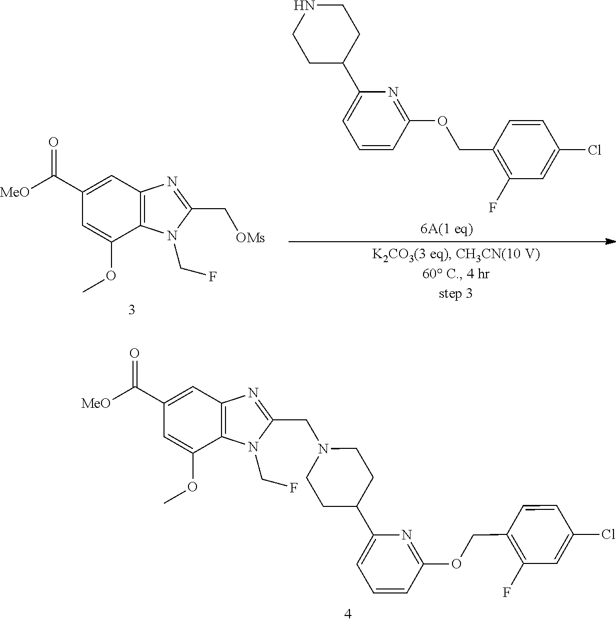 [SMILES: COC(=O)c1cc(OC)c2c(c1)nc(COS(C)(=O)=O)n2CF ; Fc1cc(Cl)ccc1COc1cccc(C2CCNCC2)n1 ; COC(=O)c1cc(OC)c2c(c1)nc(CN1CCC(c3cccc(OCc4ccc(Cl)cc4F)n3)CC1)n2CF]

To a solution of 3 (180 mg, 519.73 μmol, 1 eq) in ACN (2 mL) was added K₂CO₃ (215.49 mg, 1.56 mmol, 3 eq) and 6A (166.72 mg, 519.73 μmol, 1 eq). The mixture was stirred at 60° C. for 4 hr. LCMS showed 3 was consumed completely and one main peak with desired mass was detected. The reaction mixture was diluted with H₂O (10 mL) and extracted with DCM (10 mL*3). The combined organic layers were washed with brine (10 mL*3), dried over Na₂SO₄, filtered, and concentrated under reduced pressure to give a residue. The residue was purified by column chromatography (SiO₂, Petroleum ether/Ethyl acetate=10/1 to 0/1). 4 (140 mg, 245.18 μmol, 47.17% yield) was obtained as yellow oil.

LCMS: RT=0.511 min, MS cal.: 570.2, [M+H]⁺=571.3

¹H NMR (400 MHz, DMSO-d6) δ=8.11 (d, J=1.2 Hz, 1H), 7.96-7.89 (m, 2H), 7.83 (t, J=8.0 Hz, 1H), 7.79 (s, 1H), 7.66 (s, 1H), 7.65-7.62 (m, 1H), 7.62-7.58 (m, 1H), 7.53 (s, 1H), 7.49 (dd, J=2.0, 10.0 Hz, 1H), 7.44-7.38 (m, 1H), 7.32 (dd, J=1.6, 8.3 Hz, 1H), 6.88 (d, J=8.0 Hz, 1H), 5.52 (s, 2H), 4.46 (s, 2H), 3.90 (d, J=2.0 Hz, 6H)

### General Procedure for Preparation of Compound 192 (5):

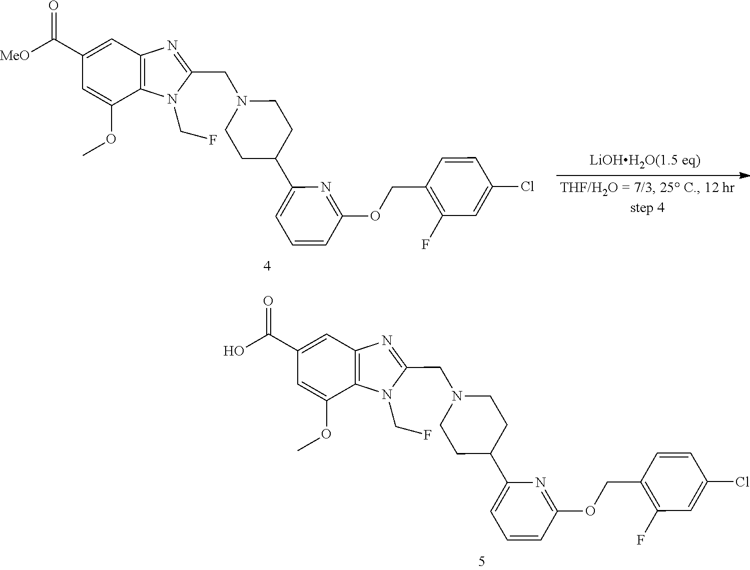 [SMILES: COC(=O)c1cc(OC)c2c(c1)nc(CN1CCC(c3cccc(OCc4ccc(Cl)cc4F)n3)CC1)n2CF ; COc1cc(C(=O)O)cc2nc(CN3CCC(c4cccc(OCc5ccc(Cl)cc5F)n4)CC3)n(CF)c12]

To a solution of 4 (60 mg, 105.08 μmol, 1 eq) in THF (0.7 mL) and H₂O (0.3 mL) was added LiOH·H₂O (6.61 mg, 157.61 μmol, 1.5 eq). The mixture was stirred at 25° C. for 12 hr. LCMS showed 4 was consumed completely and one main peak with desired mass was detected. The reaction mixture was filtered and concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 20%-60% B over 8.0 min). 2-((4-(6-((4-Chloro-2-fluorobenzyl)oxy)pyridin-2-yl)piperidin-1-yl)methyl)-1-(fluoromethyl)-7-methoxy-1H-benzo[d]imidazole-5-carboxylic acid (18.94 mg, 34.00 μmol, 32.36% yield, 100% purity) was obtained as a white solid.

LCMS: RT=2.919 min, MS cal.: 556.2, [M+H]⁺=557.2

HPLC: RT=11.836 min, purity: 100%

¹H NMR (400 MHz, DMSO-d6) δ=7.84 (s, 1H), 7.62 (t, J=8.0 Hz, 1H), 7.57 (s, 1H), 7.48-7.43 (m, 2H), 7.30 (br d, J=2.0 Hz, 1H), 6.86 (d, J=7.2 Hz, 1H), 6.70-6.64 (m, 2H), 6.55 (s, 1H), 5.36 (s, 2H), 3.99 (s, 3H), 3.88 (s, 2H), 2.92 (br d, J=11.2 Hz, 2H), 2.61-2.56 (m, 1H), 2.19 (br d, J=2.4 Hz, 2H), 1.77 (br s, 4H)

### Example A120—Preparation of Compound 194. 2-((4-(4-Chloro-3-((4-chloro-2-fluorobenzyl)oxy)phenyl)piperidin-1-yl)methyl)-4-(difluoromethoxy)-1-methyl-1H-benzo[d]imidazole-6-carboxylic acid (Compound 194) (7)

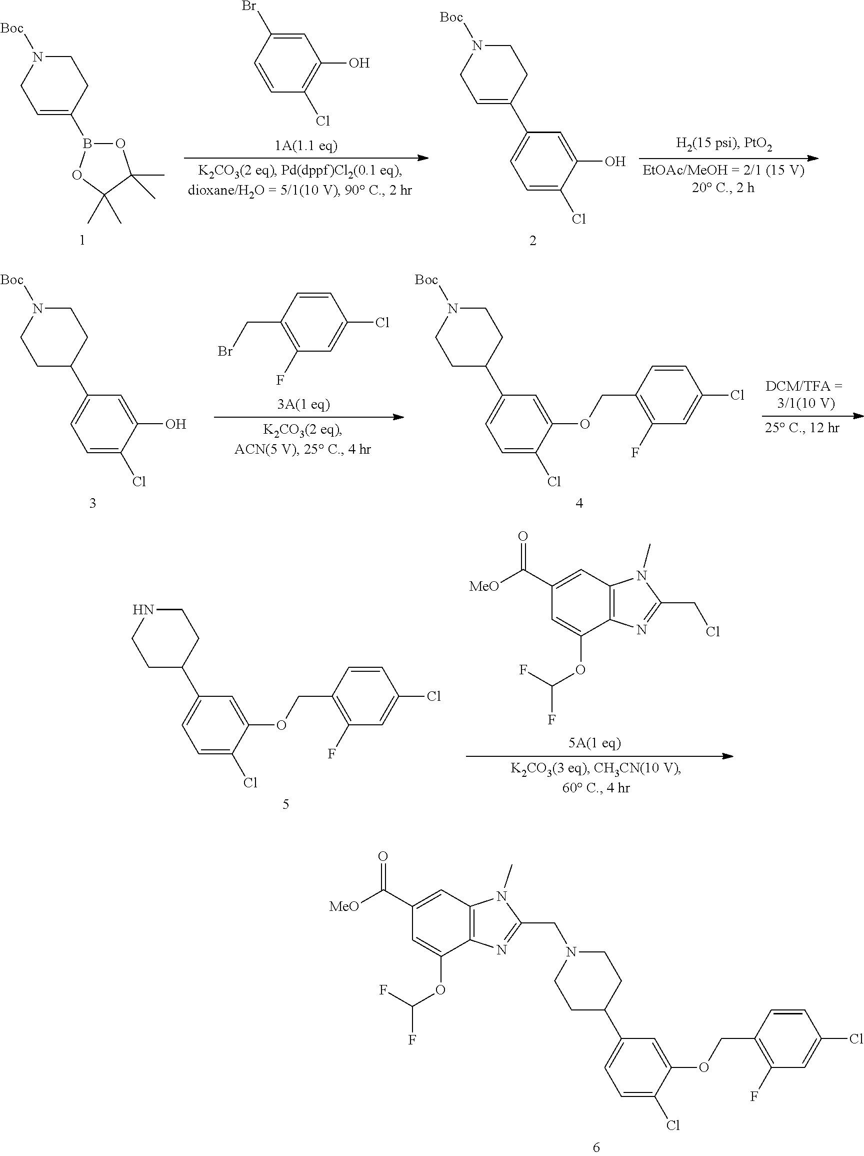

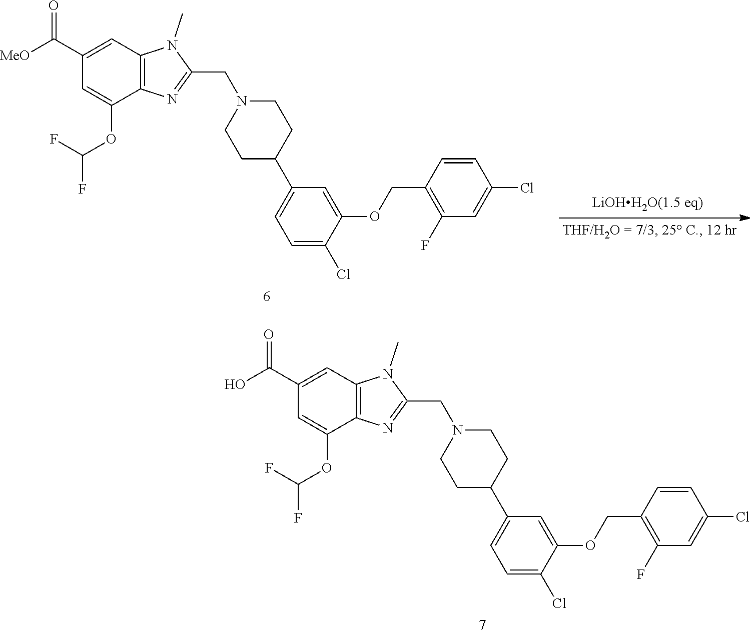 [SMILES: COC(=O)c1cc(OC(F)F)c2nc(CN3CCC(c4ccc(Cl)c(OCc5ccc(Cl)cc5F)c4)CC3)n(C)c2c1 ; Cn1c(CN2CCC(c3ccc(Cl)c(OCc4ccc(Cl)cc4F)c3)CC2)nc2c(OC(F)F)cc(C(=O)O)cc21]

### General Procedure for Preparation of Intermediate 2:

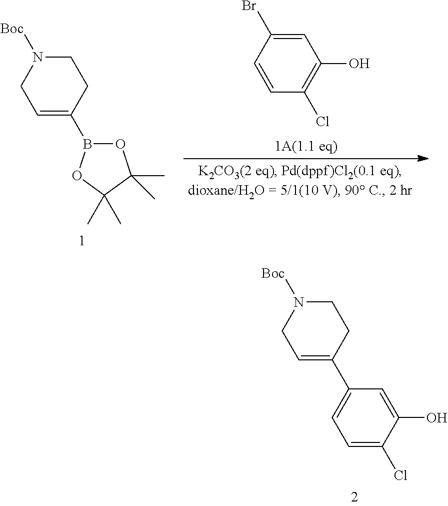 [SMILES: CC(C)(C)OC(=O)N1CC=C(B2OC(C)(C)C(C)(C)O2)CC1 ; Oc1cc(Br)ccc1Cl ; CC(C)(C)OC(=O)N1CC=C(c2ccc(Cl)c(O)c2)CC1]

A mixture of 1 (2.98 g, 9.64 mmol, 1 eq), 1A (2 g, 9.64 mmol, 1 eq), K₂CO₃ (2.66 g, 19.28 mmol, 2 eq), Pd(dppf)Cl₂ (705.42 mg, 964.08 μmol, 0.1 eq) in dioxane (20 mL) and H₂O (4 mL) was degassed and purged with N₂ for 3 times, and then the mixture was stirred at 90° C. for 2 hr under N₂ atmosphere. TLC (Petroleum ether:Ethyl acetate=5:1, Rf=0.29) indicated 1 was consumed completely and one new spot formed. The reaction was clean according to TLC. The suspension was filtered through a pad of Celite, and filter cake was washed with EtOAc (20 mL*3). The reaction mixture was diluted with H₂O 30 mL and extracted with EtOAc 60 mL (20 mL*3). The combined organic layers were washed with brine 15 mL, dried over Na₂SO₄, filtered, and concentrated under reduced pressure to give a residue. The residue was purified by column chromatography (SiO₂, Petroleum ether/Ethyl acetate=10/1 to 3/1). 2 (2.9 g, 9.36 mmol, 97.10% yield) was obtained as a white solid.

LCMS: RT=1.368 min, MS cal.: 309.11, [M−H]⁺=308.0

¹H NMR (400 MHz, CDCl₃) δ=7.29 (d, J=1.2 Hz, 1H), 7.05 (d, J=2.0 Hz, 1H), 6.91 (dd, J=2.0, 8.0 Hz, 1H), 6.05 (br s, 1H), 5.82 (br s, 1H), 4.09 (br d, J=2.8 Hz, 2H), 3.65 (t, J=5.6 Hz, 2H), 2.49 (br d, J=1.2 Hz, 2H), 1.52 (s, 9H)

### General Procedure for Preparation of Intermediate 3:

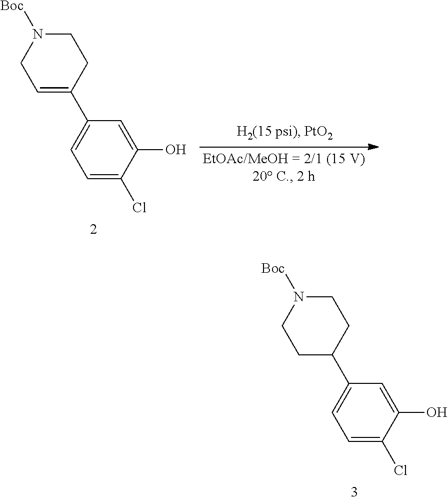 [SMILES: CC(C)(C)OC(=O)N1CC=C(c2ccc(Cl)c(O)c2)CC1 ; CC(C)(C)OC(=O)N1CCC(c2ccc(Cl)c(O)c2)CC1]

EtOAc (4 mL) and MeOH (2 mL) was charged to the round bottom flask, then 2 (400 mg, 1.29 mmol, 1 eq) was added to the mixture at 20° C. At 20° C., PtO₂ (200 mg) under N₂ atmosphere. The suspension was degassed and purged with H₂ for 3 times. The mixture was stirred under H₂ (15 Psi) at 20° C. for 2 hr. LC-MS showed 2 was consumed completely and desired mass was detected. The mixture was filtered through a Celite pad and washed by MeOH (5 mL*3), and the filtrate was concentrated to give the crude product. 3 (240 mg, 769.72 μmol, 59.61% yield) was obtained as a white solid.

LCMS: RT=1.921 min, MS cal.: 311.1/313.1, [M−(t-Bu)+CH₃CN+H]⁺=297.1/299.1

1H NMR (400 MHz, MeOH-d4) δ=7.20 (d, J=8.2 Hz, 1H), 6.79 (d, J=2.0 Hz, 1H), 6.70 (dd, J=2.0, 8.0 Hz, 1H), 4.21 (br d, J=13.2 Hz, 2H), 2.86 (br s, 2H), 2.65 (tt, J=3.6, 12.0 Hz, 1H), 1.81 (br d, J=13.2 Hz, 2H), 1.61-1.44 (m, 4H), 1.49 (s, 1H)

### General Procedure for Preparation of Compound 4:

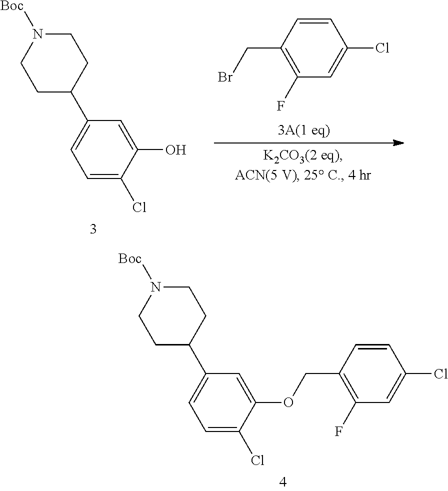 [SMILES: CC(C)(C)OC(=O)N1CCC(c2ccc(Cl)c(O)c2)CC1 ; Fc1cc(Cl)ccc1CBr ; CC(C)(C)OC(=O)N1CCC(c2ccc(Cl)c(OCc3ccc(Cl)cc3F)c2)CC1]

A mixture of 3 (200 mg, 641.43 μmol, 1 eq), 3A (143.34 mg, 641.43 μmol, 1 eq), K₂CO₃ (177.30 mg, 1.28 mmol, 2 eq) in ACN (4 mL) was degassed and purged with N₂ for 3 times, and then the mixture was stirred at 25° C. for 4 hr under N₂ atmosphere. LC-MS (product, Rt=2.741 min) showed 3 was consumed completely and desired mass was detected. The reaction mixture was diluted with H₂O 15 mL and extracted with EtOAc 45 mL (15 mL*3). The combined organic layers were washed with brine 15 mL, dried over Na₂SO₄, filtered and concentrated under reduced pressure to give a residue. 4 (230 mg, 506.21 μmol, 78.92% yield) was obtained as a colorless oil.

LCMS: RT=2.741 min, MS cal.: 453.13, [M−100]⁺=354.1

¹H NMR (400 MHz, CDCl₃) δ=7.57 (t, J=8.0 Hz, 1H), 7.31 (d, J=8.0 Hz, 1H), 7.20 (dd, J=1.6, 8.4 Hz, 1H), 7.14 (dd, J=2.0, 9.6 Hz, 1H), 6.84 (d, J=1.6 Hz, 1H), 6.79 (dd, J=1.6, 8.1 Hz, 1H), 5.16 (s, 2H), 4.25 (br d, J=13.2 Hz, 2H), 2.80 (dt, J=2.4, 12.8 Hz, 2H), 2.62 (tt, J=3.2, 12.4 Hz, 1H), 1.80 (br d, J=12.4 Hz, 2H), 1.68-1.56 (m, 2H), 1.49 (s, 9H)

### General Procedure for Preparation of Intermediate 5:

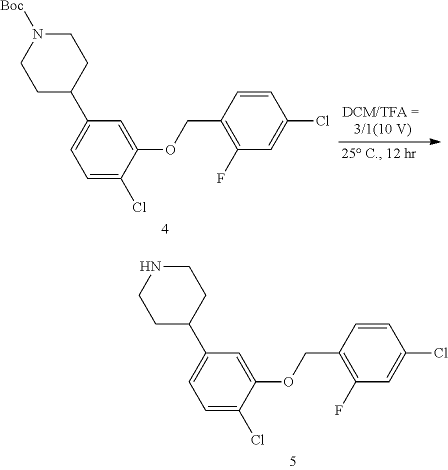 [SMILES: CC(C)(C)OC(=O)N1CCC(c2ccc(Cl)c(OCc3ccc(Cl)cc3F)c2)CC1 ; Fc1cc(Cl)ccc1COc1cc(C2CCNCC2)ccc1Cl]

A mixture of 4 (200 mg, 440.18 μmol, 1 eq), TFA (767.50 mg, 6.73 mmol, 0.5 mL, 15.29 eq) in DCM (1.5 mL) was degassed and purged with N₂ for 3 times, and then the mixture was stirred at 25° C. for 12 hr under N₂ atmosphere. LC-MS (product, RT=1.454 min) showed 4 was consumed completely and one main peak with desired mass was detected. The reaction mixture was concentrated under reduced pressure to give a residue and without purification. 5 (200 mg, 427.11 μmol, 97.03% yield, TFA) was obtained as a white solid.

LCMS: RT=1.454 min, MS cal.: 353.07, [M−100]⁺=354.1

¹H NMR (400 MHz, CDCl₃) δ=9.06-8.80 (m, 1H), 8.61-8.35 (m, 1H), 7.56 (t, J=8.0 Hz, 1H), 7.36 (d, J=8.0 Hz, 1H), 7.20 (d, J=8.4 Hz, 1H), 7.14 (dd, J=1.6, 10.0 Hz, 1H), 7.04 (br s, 2H), 6.87 (d, J=1.2 Hz, 1H), 6.80 (dd, J=1.6, 8.0 Hz, 1H), 5.17 (s, 2H), 3.62 (br d, J=12.0 Hz, 2H), 3.09 (br s, 2H), 2.85-2.73 (m, 1H), 2.08 (br s, 1H), 2.11-1.96 (m, 1H)

### General Procedure for Preparation of Intermediate 6:

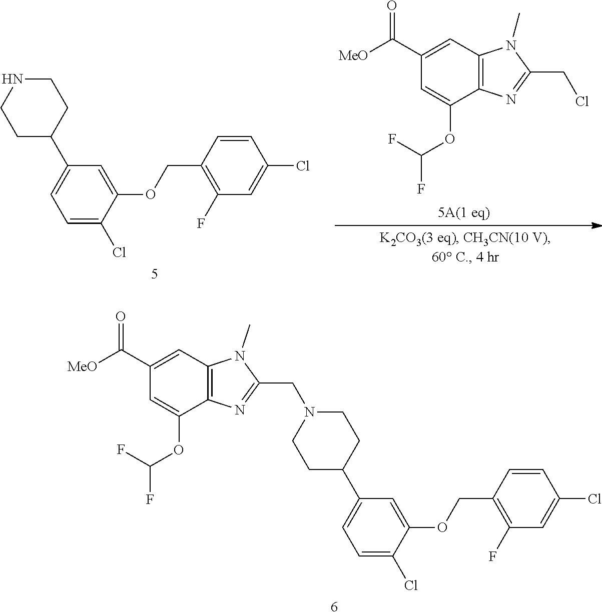 [SMILES: Fc1cc(Cl)ccc1COc1cc(C2CCNCC2)ccc1Cl ; COC(=O)c1cc(OC(F)F)c2nc(CCl)n(C)c2c1 ; COC(=O)c1cc(OC(F)F)c2nc(CN3CCC(c4ccc(Cl)c(OCc5ccc(Cl)cc5F)c4)CC3)n(C)c2c1]

A mixture of 5 (200 mg, 564.58 μmol, 1 eq), 5A (137.61 mg, 451.66 μmol, 0.8 eq), K₂CO₃ (234.08 mg, 1.69 mmol, 3 eq) in CH₃CN (2 mL) was degassed and purged with N₂ for 3 times, and then the mixture was stirred at 60° C. for 4 hr under N₂ atmosphere. LC-MS (product, RT=1.883 min) showed 5 was consumed completely and one main peak with desired mass was detected. The reaction mixture was diluted with H₂O 20 mL and extracted with EtOAc 60 mL (20 mL*3). The combined organic layers were washed with brine 15 mL, dried over Na₂SO₄, filtered, and concentrated under reduced pressure to give a residue. The residue was purified by column chromatography (SiO₂, Petroleum ether/Ethyl acetate=10/1 to 1/1). 6 (80 mg, 128.52 μmol, 22.76% yield) was obtained as a white solid.

LCMS: RT=1.883 min, MS cal.: 621.14, [M+1]⁺=622.2

¹H NMR (400 MHz, DMSO-d6) δ=8.12 (d, J=1.2 Hz, 1H), 7.65 (t, J=74.4 Hz 1H), 7.61 (t, J=8.2 Hz, 1H), 7.54-7.52 (m, 1H), 7.52-7.48 (m, 1H), 7.38-7.30 (m, 2H), 7.17 (d, J=1.2 Hz, 1H), 6.86 (dd, J=1.6, 8.4 Hz, 1H), 5.22 (s, 2H), 3.98 (s, 3H), 3.90 (s, 3H), 3.88-3.85 (m, 2H), 3.02-3.00 (m, 1H), 2.96 (br d, J=11.0 Hz, 2H), 2.22 (br t, J=10.4 Hz, 2H), 1.78-1.62 (m, 4H)

### General Procedure for Preparation of Compound 194 (7):

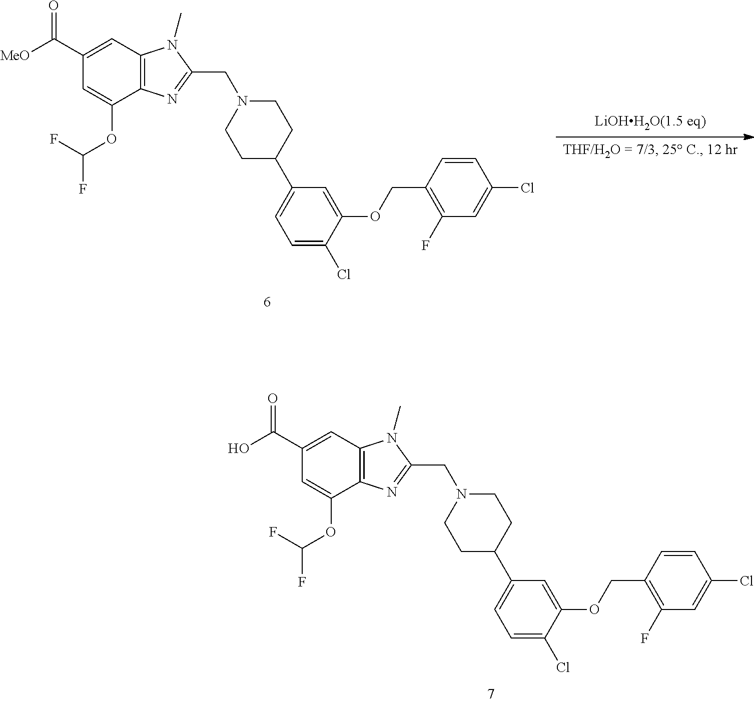

A mixture of 6 (60 mg, 96.39 μmol, 1 eq), LiOH·H₂O (6.07 mg, 144.59 μmol, 1.5 eq), in THF (0.7 mL) and H₂O (0.3 mL) was degassed and purged with N₂ for 3 times, and then the mixture was stirred at 25° C. for 12 hr under N₂ atmosphere. LC-MS (product, RT=1.301 min) showed 6 was consumed completely and one main peak with desired mass was detected. The reaction was directly purified. The residue was purified by prep-HPLC (column: Waters Xbridge Prep OBD C18 150*40 mm*10 um; mobile phase: [H₂O (10 mM NH₄HCO₃)-ACN]; gradient: 15%-65% B over 8.0 min). 2-((4-(4-Chloro-3-((4-chloro-2-fluorobenzyl)oxy)phenyl)piperidin-1-yl)methyl)-4-(difluoromethoxy)-1-methyl-1H-benzo[d]imidazole-6-carboxylic acid (29.69 mg, 48.55 μmol, 50.37% yield, 99.49% purity) was obtained as a white solid.

LCMS: RT=2.628 min, MS cal.: 607.13, [M+1]⁺=608.2

HPLC: RT=13.56 min, purity: 99.49%

¹H NMR (400 MHz, MeOD) δ=8.11 (d, J=1.2 Hz, 1H), 7.69 (s, 1H), 7.57 (t, J=8.4 Hz, 1H), 7.30-7.22 (m, 4H), 7.25 (t, J=74.4 Hz, 1H), 7.01 (d, J=1.6 Hz, 1H), 6.86 (dd, J=1.6, 8.3 Hz, 1H), 5.18 (s, 2H), 4.02 (s, 3H), 3.96 (s, 2H), 3.07 (br d, J=11.2 Hz, 2H), 2.65-2.53 (m, 1H), 2.44-2.31 (m, 2H), 1.89-1.70 (m, 4H)

### Example A121—Preparation of Compound 195. 2-((4-(6-((4-Chloro-2-fluorophenyl)methoxy-d2)pyridin-2-yl)piperidin-1-yl)methyl)-4-(difluoromethoxy)-1-methyl-1H-benzo[d]imidazole-6-carboxylic acid (Compound 195) (6)

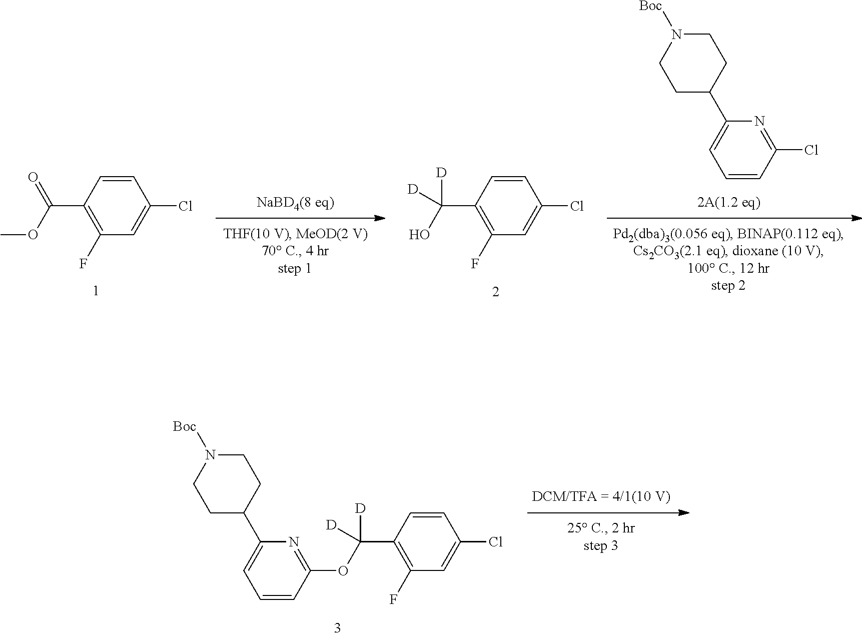 [SMILES: COC(=O)c1ccc(Cl)cc1F ; [2H]C([2H])(O)c1ccc(Cl)cc1F ; CC(C)(C)OC(=O)N1CCC(c2cccc(Cl)n2)CC1 ; [2H]C([2H])(Oc1cccc(C2CCN(C(=O)OC(C)(C)C)CC2)n1)c1ccc(Cl)cc1F]

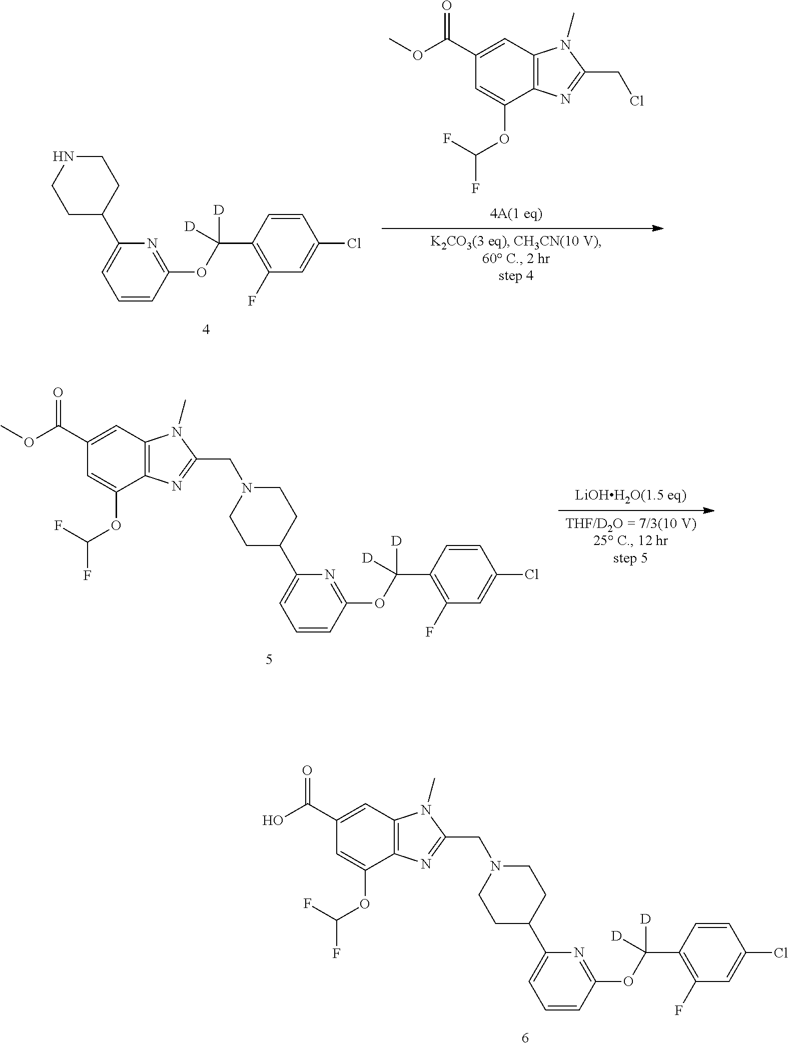

### General Procedure for Preparation of Intermediate 2:

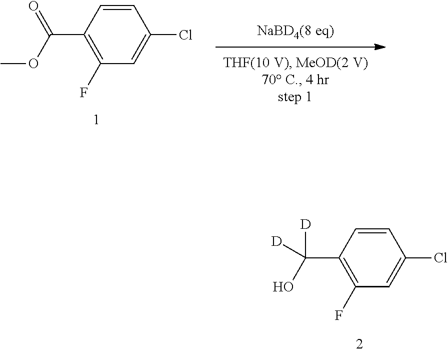

A mixture of 1 (500 mg, 2.65 mmol, 1 eq) in THF (5 mL) was added NaBD₄ (802.46 mg, 21.21 mmol, 8 eq) at 25° C., and the mixture was stirred at 70° C. for 10 min. Then MeOD (1.25 mL) was added at 70° C. for 1 hour. The mixture was stirred at 70° C. for 4 hr. TLC (SiO₂, Petroleum ether:Ethyl acetate=3:1, R_{f}=0.50) showed the reaction was completed. The residue was poured into water (50 mL). The aqueous phase was extracted with dichloromethane (20 mL*3). The combined organic phase was washed with saturated brine (50 mL), dried with anhydrous Na₂SO₄, filtered, and concentrated in vacuum. The residue was purified by column chromatography (SiO₂, PE/EtOAc=1/0 to 0/1). 2 (340 mg, 1.93 mmol, 73% yield) was obtained as a yellow oil.

HPLC: product: RT=2.214 min, purity: 92.52%

1H NMR (400 MHz, DMSO-d6) δ=7.48 (t, J=8.0 Hz, 1H), 7.35 (dd, J=2.0, 10.0 Hz, 1H), 7.31-7.24 (m, 1H), 5.30 (s, 1H)

### General Procedure for Preparation of Intermediate 3:

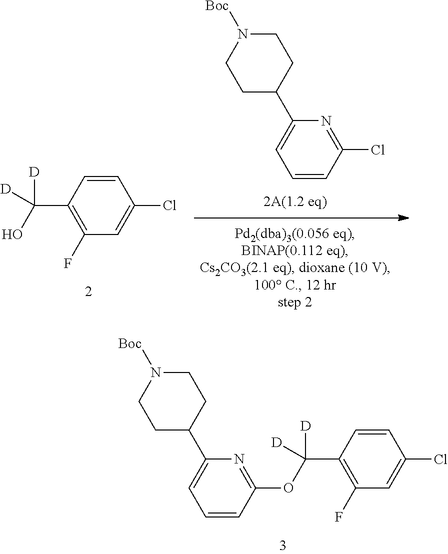 [SMILES: [2H]C([2H])(O)c1ccc(Cl)cc1F ; CC(C)(C)OC(=O)N1CCC(c2cccc(Cl)n2)CC1 ; [2H]C([2H])(Oc1cccc(C2CCN(C(=O)OC(C)(C)C)CC2)n1)c1ccc(Cl)cc1F]

To a mixture of 2 (340 mg, 2.09 mmol, 1 eq), 2A (682.72 mg, 2.30 mmol, 1.1 eq), Cs₂CO₃ (1.43 g, 4.39 mmol, 2.1 eq) and BINAP (145.84 mg, 234.22 μmol, 0.112 eq) in dioxane (5 mL) was added Pd₂(dba)₃ (107.24 mg, 117.11 μmol, 0.056 eq) at 25° C. The mixture was degassed under reduced pressure and recharged with N₂. The mixture was stirred at 100° C. for 12 hours. LC-MS showed 2 was consumed completely and one main peak with desired mass was detected. The residue was poured into water (100 mL). The aqueous phase was extracted with ethyl acetate (20 mL*3). The combined organic phase was washed with saturated brine (100 mL), dried with anhydrous Na₂SO₄, filtered, and concentrated in vacuum. The residue was purified by column chromatography (SiO₂, Petroleum ether:Ethyl acetate=1/0 to 2/1). 3 (680 mg, 1.45 mmol, 69.38% yield) was obtained as a colorless oil.

LCMS: RT=1.786 min, MS cal.: 422.2, [M+H]⁺=423.0

HPLC: product: RT=4.131 min, purity: 90.23%

¹H NMR (400 MHz, DMSO-d6) δ=7.63 (dd, J=7.2, 8.0 Hz, 1H), 7.55 (t, J=8.4 Hz, 1H), 7.44 (dd, J=2.0, 10.0 Hz, 1H), 7.29 (dd, J=2.0, 8.4 Hz, 1H), 6.87 (d, J=7.2 Hz, 1H), 6.68 (d, J=8.0 Hz, 1H), 4.10-3.97 (m, 2H), 2.93-2.70 (m, 3H), 1.76 (d, J=10.8 Hz, 2H), 1.54 (dq, J=4.0, 12.4 Hz, 2H), 1.42 (s, 9H)

### General Procedure for Preparation of Intermediate 4:

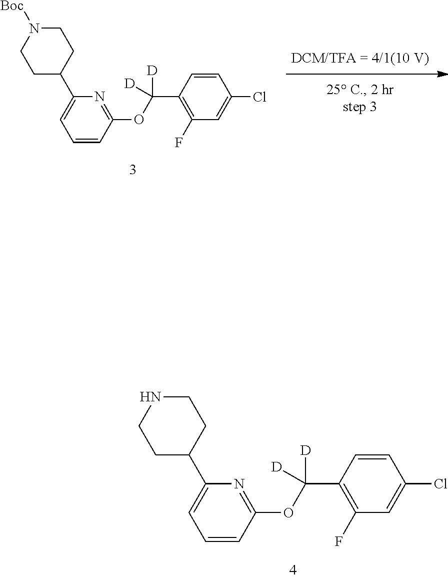 [SMILES: [2H]C([2H])(Oc1cccc(C2CCN(C(=O)OC(C)(C)C)CC2)n1)c1ccc(Cl)cc1F ; [2H]C([2H])(Oc1cccc(C2CCNCC2)n1)c1ccc(Cl)cc1F]

To a solution of 3 (630 mg, 1.49 mmol, 1 eq) in DCM (6 mL) was added TFA (2.30 g, 20.19 mmol, 1.5 mL, 13.56 eq). The mixture was stirred at 25° C. for 2 hr. LC-MS showed 3 was consumed completely and one main peak with desired mass was detected. The mixture was filtered and concentrated in vacuum. The crude product was used into the next step without further purification. 4 (550 mg, crude) was obtained as a yellow solid.

LCMS: RT=0.392 min, MS cal.: 322.1, [M+H]⁺=323.1

HPLC: product: RT=2.454 min, purity: 97.38%

¹H NMR (400 MHz, MeOH-d4) δ=7.64 (dd, J=7.6, 8.0 Hz, 1H), 7.50 (t, J=8.0 Hz, 1H), 7.28-7.18 (m, 2H), 6.88 (d, J=7.2 Hz, 1H), 6.71 (d, J=8.0 Hz, 1H), 3.51-3.41 (m, 2H), 3.10 (dt, J=3.6, 12.4 Hz, 2H), 2.97 (tt, J=4.0, 11.2 Hz, 1H), 2.14-1.95 (m, 4H)

### General Procedure for Preparation of Intermediate 5:

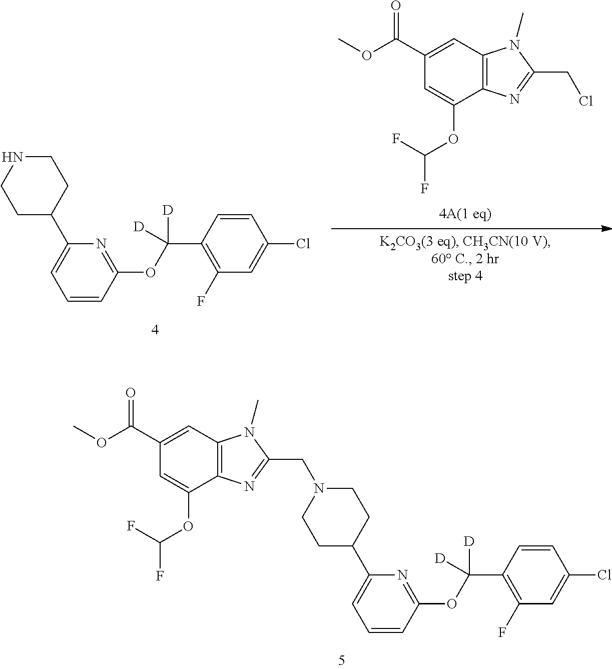 [SMILES: [2H]C([2H])(Oc1cccc(C2CCNCC2)n1)c1ccc(Cl)cc1F ; COC(=O)c1cc(OC(F)F)c2nc(CCl)n(C)c2c1 ; "4A(1 eq)" ; "K2CO3(3 eq), CH3CN(10 V),60° C., 2 hrstep 4" ; [2H]C([2H])(Oc1cccc(C2CCN(Cc3nc4c(OC(F)F)cc(C(=O)OC)cc4n3C)CC2)n1)c1ccc(Cl)cc1F]

To a solution of 4A (60 mg, 196.93 μmol, 1 eq) in ACN (1 mL) was added K₂CO₃ (81.65 mg, 590.79 μmol, 3 eq) and 4 (82.64 mg, 256.01 μmol, 1.3 eq). The mixture was de-gassed under reduced pressure and recharged with N₂. The mixture was stirred at 60° C. for 2 hr. LC-MS showed 4A was consumed completely and one main peak with desired mass was detected. The mixture was filtered and concentrated in vacuum. The residue was purified by prep-TLC (SiO₂, Petroleum ether:Ethyl acetate=1:1, R_{f}=0.50). 5 (115 mg, 191.70 μmol, 97.34% yield) was obtained as a yellow solid.

LCMS: RT=0.515 min, MS cal.: 590.2 [M+H]⁺=591.3

HPLC: product: RT=2.991 min, purity: 98.52%

¹H NMR (400 MHz, DMSO-d6) δ=8.12 (s, 1H), 7.65 (t, J=74.4 Hz, 1H), 7.62 (m, 1H), 7.58-7.52 (m, 2H), 7.47-7.42 (m, 1H), 7.28 (dd, J=2.0, 8.4 Hz, 1H), 6.86 (d, J=7.2 Hz, 1H), 6.66 (d, J=8.0 Hz, 1H), 3.98 (s, 3H), 3.90 (s, 3H), 3.86 (s, 2H), 2.94 (d, J=11.2 Hz, 2H), 2.64-2.54 (m, 1H), 2.22 (t, J=10.0 Hz, 2H), 1.82-1.64 (m, 4H)

### General Procedure for Preparation of Compound 195 (6):

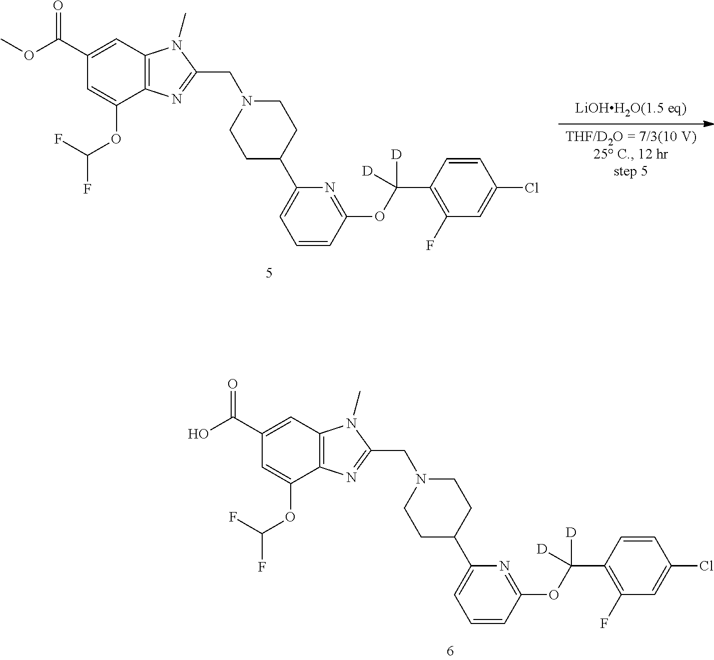

To a solution of 5 (90 mg, 152.28 μmol, 1 eq) in THF (0.7 mL) and H₂O (0.3 mL) was added LiOH H₂O (9.59 mg, 228.42 μmol, 1.5 eq). The mixture was stirred at 25° C. for 15 hr. LC-MS showed 5 was consumed completely and one main peak with desired mass was detected. The mixture was filtered and concentrated in vacuum. The residue was purified by prep-HPLC (column: Waters Xbridge Prep OBD C₁₈ 150*40 mm*10 um; mobile phase: [H₂O (10 mM NH₄HCO₃)-ACN]; gradient: 20%-70% B over 8.0 min). 2-((4-(6-((4-Chloro-2-fluorophenyl)methoxy-d2)pyridin-2-yl)piperidin-1-yl)methyl)-4-(difluoromethoxy)-1-methyl-1H-benzo[d]imidazole-6-carboxylic acid (28.74 mg, 49.78 μmol, 32.69% yield) was obtained as a white solid.

LCMS: RT=1.179 min, MS cal.: 576.2, [M+H]⁺=577.3

LCMS: RT=2.795 min, MS cal.: 576.2, [M+H]⁺=577.2

HPLC: product: RT=12.353 min, purity: 99.94%

¹H NMR (400 MHz, MeOH-d4) δ=8.09 (d, J=1.2 Hz, 1H), 7.68 (s, 1H), 7.60-7.53 (m, 1H), 7.48 (t, J=8.0 Hz, 1H), 7.25 (t, J=74.4 Hz, 1H), 7.22-7.14 (m, 2H), 6.82 (d, J=7.6 Hz, 1H), 6.62 (d, J=8.4 Hz, 1H), 4.01 (s, 3H), 3.99 (s, 2H), 3.11 (d, J=11.4 Hz, 2H), 2.67 (td, J=8.0, 15.6 Hz, 1H), 2.49-2.36 (m, 2H), 1.97-1.83 (m, 4H)

### Example A122—Preparation of Compound 196. 2-((4-((6-((4-Chloro-2-fluorophenoxy)methyl)pyridin-2-yl)oxy)piperidin-1-yl)methyl)-4-(difluoromethoxy)-1-methyl-1H-benzo[d]imidazole-6-carboxylic acid (Compound 196) (6)

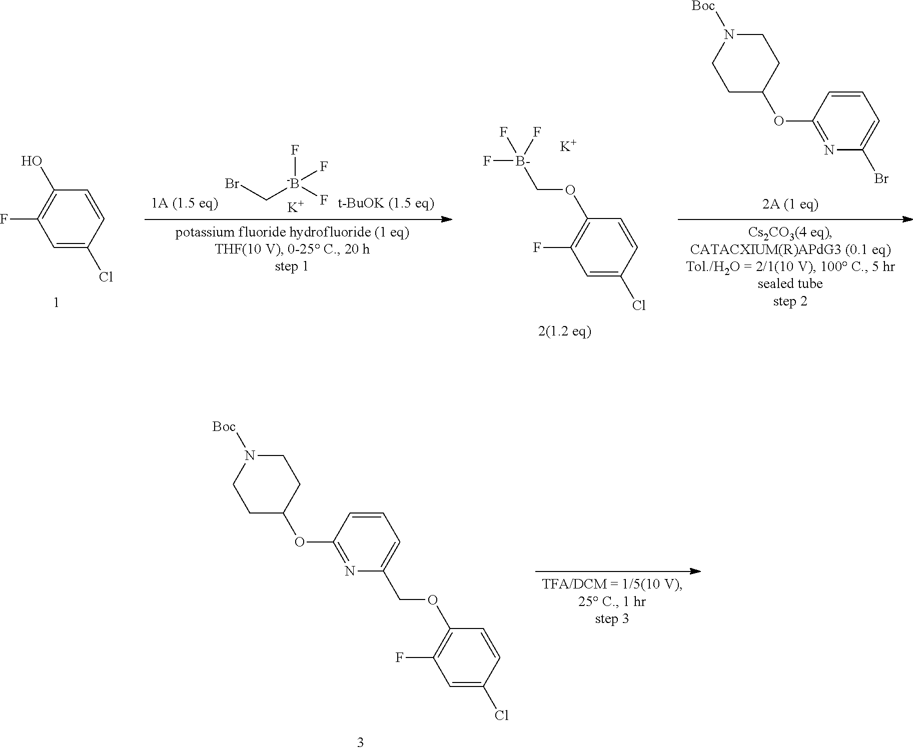

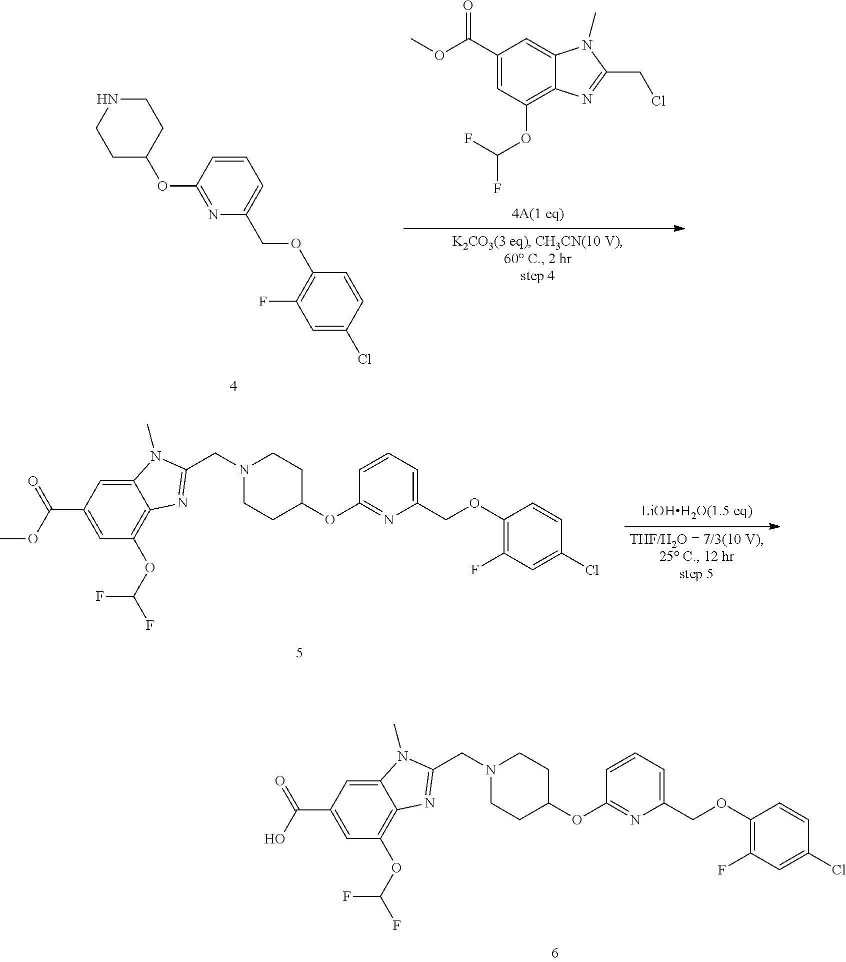

### General Procedure for Preparation of Intermediate 2:

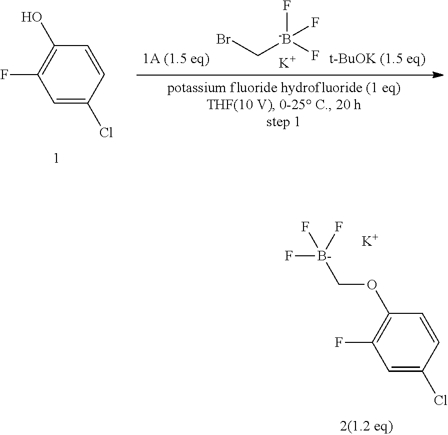

To a solution of t-BuOK (1 M, 40.94 mL, 1.5 eq) in THF was added 1 (4 g, 27.30 mmol, 1 eq) at 0° C. The mixture was stirred at 0° C. for 0.1 h. Then the mixture was warmed to 25° C. and stirred for 0.1 h. Then 1A (8.22 g, 40.94 mmol, 1.5 eq) was added to the mixture at 0° C. The mixture was stirred at 25° C. for 20 h. TLC (Petroleum ether/Ethyl acetate=3:1, Rf=0.6) indicated 1 was consumed completely. The mixture added a solution of KHF₂ (532.96 mg) at 25° C., the mixture was stirred at 25° C. for 0.5 h. The reaction mixture was concentrated under reduced pressure to remove solvent. The residue was triturated with MTBE (30 mL). The mixture was filtered and collected. The final product was dried under vacuum at 50° C. for 30 mins. The residue was triturated with H₂O (15 mL). The mixture was filtered and collected. The final product was dried under vacuum at 50° C. for 30 mins. 2 (4.5 g, 15.20 mmol, 55.68% yield, 90% purity, K⁺) was obtained as a white solid.

LCMS: RT=0.904 min, MS cal.: 227.01, [M−H]⁺=226.9

¹H NMR (400 MHz, DMSO-d₆) δ=7.28-7.24 (m, 1H), 7.11-7.07 (m, 2H), 3.05 (q, J=5.2 Hz, 2H).

### General Procedure for Preparation of Intermediate 3:

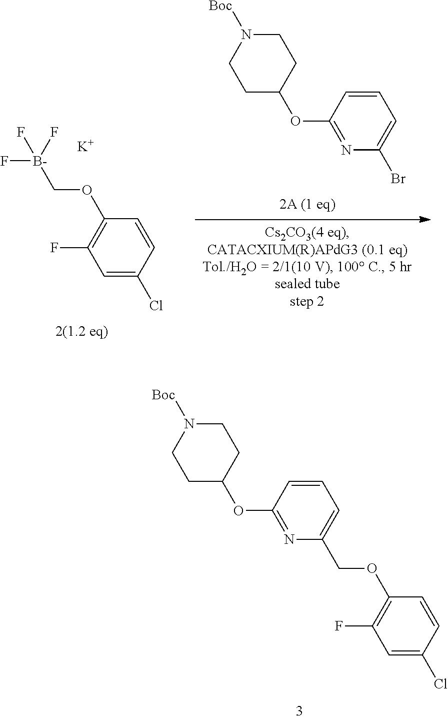

To a solution of 2A (0.5 g, 1.40 mmol, 1 eq) and 2 (447.54 mg, 1.68 mmol, 1.2 eq, K⁺) in Toluene (4 mL) and was added Cs₂CO₃ (1.82 g, 5.60 mmol, 4 eq) in H₂O (2 mL) and CATACXIUM® A Pd G₃ (101.93 mg, 139.96 μmol, 0.1 eq). The mixture was stirred at 100° C. for 5 hr. LCMS showed ˜10% of 2A remained. On LC-MS, ˜38% of desired was detected. The reaction mixture was diluted with H₂O (40 mL) and extracted with EtOAc (150 mL). The combined organic layers were washed with brine (30 mL), dried with Na₂SO₄, filtered, and concentrated under reduced pressure to give a residue. The residue was purified by prep-HPLC (column: Welch Xtimate C18 250*100 mm #10 um; mobile phase: [H₂O (10 mM NH₄HCO₃)-ACN]; gradient: 60%-95% B over 20 min). 3 (0.215 g, 984.20 μmol, 35.16% yield) was obtained as a faint yellow oil.

LCMS: RT=1.735 min, MS cal.: 436.16, [M+H]⁺=437.0

### General Procedure for Preparation of Intermediate 4:

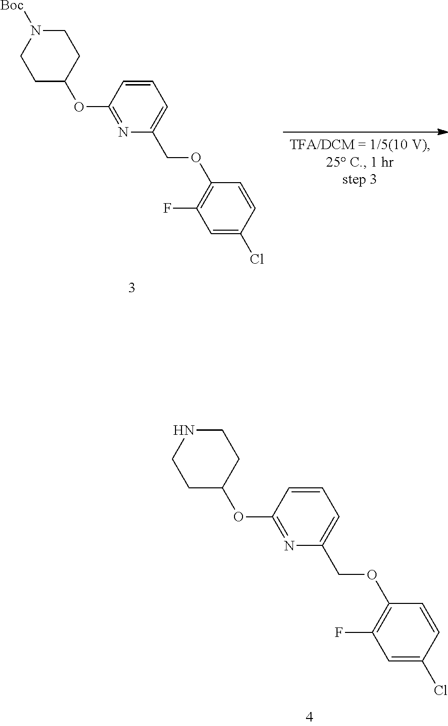

To a solution of 3 (50 mg, 114.44 μmol, 1 eq) in DCM (4 mL) was added TFA (0.8 mL). The mixture was stirred at 25° C. for 1 hr. LC-MS showed 3 was consumed completely and one main peak with desired mass was detected. The mixture was adjusted to pH=7-8 with a.q. Na₂CO₃. The mixture was diluted with H₂O (20 mL) and extracted with DCM 30 mL (10 mL*3). The organic phase was washed with brine (20 mL), dried over Na₂SO₄, filtered, and concentrated under reduced pressure to give a residue. The organic phase was concentrated under reduced pressure at 40° C. to give a residue. 4 (270 mg, 801.69 μmol, 92.17% yield) was obtained as a yellow oil.

LCMS: RT=1.101 min, MS cal.: 336.1, 338.1, [M+H]⁺=336.9, 338.9

¹H NMR (400 MHz, CHCl3-d) δ=7.59 (t, J=7.6 Hz, 1H), 7.13 (dd, J=2.4, 10.8 Hz, 1H), 7.07-6.91 (m, 3H), 6.65 (d, J=8.4 Hz, 1H), 5.17 (tt, J=4.0, 8.0 Hz, 1H), 5.11 (s, 2H), 3.75 (br s, 2H), 3.26-3.13 (m, 2H), 2.87 (ddd, J=3.2, 8.8, 12.4 Hz, 2H), 2.14-2.02 (m, 2H), 1.76 (dtd, J=4.0, 8.8, 12.8 Hz, 2H)

### General Procedure for Preparation of Intermediate 5:

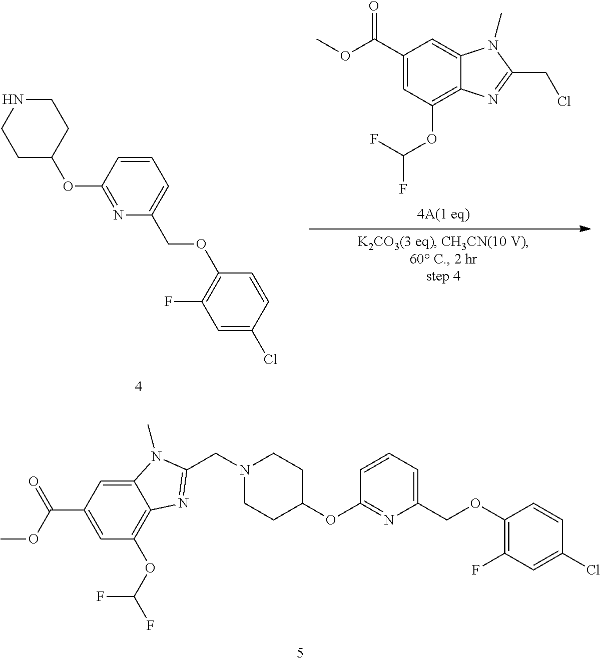 [SMILES: Fc1cc(Cl)ccc1OCc1cccc(OC2CCNCC2)n1 ; COC(=O)c1cc(OC(F)F)c2nc(CCl)n(C)c2c1 ; "4A(1 eq)" ; "K2CO3(3 eq), CH3CN(10 V),60° C., 2 hrstep 4" ; COC(=O)c1cc(OC(F)F)c2nc(CN3CCC(Oc4cccc(COc5ccc(Cl)cc5F)n4)CC3)n(C)c2c1]

To a solution of 4 (130 mg, 386.00 μmol, 1 eq) and 4A (117.61 mg, 386.00 μmol, 1 eq) in CH₃CN (2 mL) was added K₂CO₃ (160.04 mg, 1.16 mmol, 3 eq). The mixture was stirred at 60° C. for 2 hr. LC-MS showed 4 was consumed completely and one main peak with desired mass was detected. The reaction mixture was diluted with H₂O (20 mL) and extracted with EtOAc (30 mL). The combined organic layers were washed with brine (20 mL), dried with Na₂SO₄, filtered and concentrated under reduced pressure to give a residue. The residue was purified by column chromatography (SiO₂, Petroleum ether/Ethyl acetate=3/1 to 0/1). 5 (70 mg, 115.70 μmol, 29.97% yield) was obtained as a yellow solid.

LCMS: RT=1.701 min, MS cal.: 604.17, 606.17, [M+H]⁺=605.1

¹H NMR (400 MHz, DMSO-d₆) δ=8.11 (d, J=1.2 Hz, 1H), 7.71 (t, J=7.6 Hz, 1H), 7.65 (t, J=74 Hz, 1H), 7.53 (s, 1H), 7.44 (dd, J=2.4, 11.8 Hz, 1H), 7.31-7.23 (m, 1H), 7.21-7.16 (m, 1H), 7.04 (d, J=7.2 Hz, 1H), 6.71 (d, J=8.4 Hz, 1H), 5.18 (s, 2H), 4.98-4.88 (m, 1H), 3.95 (s, 3H), 3.90 (s, 3H), 3.86 (s, 2H), 2.80-2.71 (m, 2H), 2.39-2.27 (m, 2H), 1.97-1.86 (m, 2H), 1.68-1.55 (m, 2H), 1.17 (t, J=7.2 Hz, 1H)

### General Procedure for Preparation of Compound 196 (6):

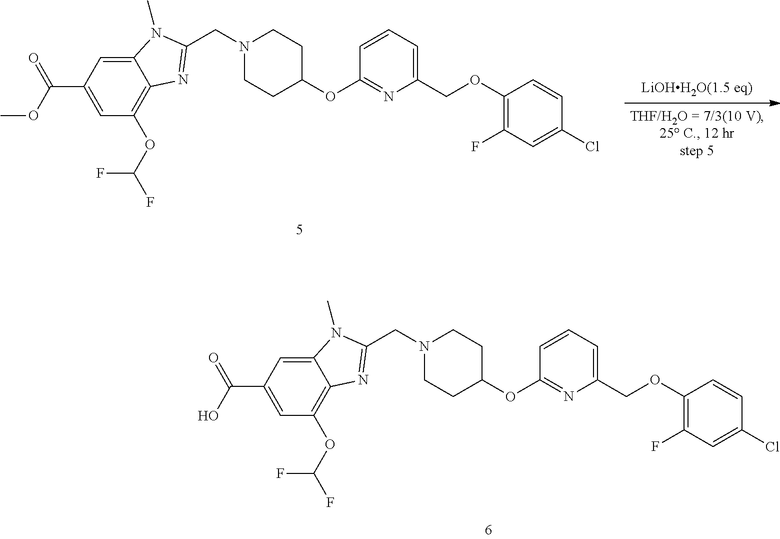

A mixture of 5 (65 mg, 107.44 μmol, 1 eq) in THF (0.7 mL) was added LiOH H₂O (6.76 mg, 161.16 μmol, 1.5 eq) in H₂O (0.3 mL). The mixture was stirred at 25° C. for 12 hr. LC-MS showed 5 was consumed completely and one main peak with desired mass was detected. The residue was purified by prep-HPLC (neutral condition; column: Waters Xbridge Prep OBD C18 150*40 mm*10 um; mobile phase: [H₂O (10 mM NH₄HCO₃)-ACN]; gradient: 15%-65% B over 8.0 min). 2-((4-((6-((4-Chloro-2-fluorophenoxy)methyl)pyridin-2-yl)oxy)piperidin-1-yl)methyl)-4-(difluoromethoxy)-1-methyl-1H-benzo[d]imidazole-6-carboxylic acid (29.48 mg, 49.38 μmol, 45.97% yield, 99% purity) was obtained as a white solid.

LCMS: RT=3.138 min, MS cal.: 590.15, 592.15, [M+H]⁺=591.1

HPLC: RT=12.391 min

¹H NMR (400 MHz, DMSO-d₆) δ=8.04 (s, 1H), 7.71 (t, J=7.6 Hz, 1H), 7.63 (t, J=74.4 Hz, 1H), 7.51 (s, 1H), 7.45 (dd, J=3.2, 10.4 Hz, 1H), 7.32-7.23 (m, 1H), 7.21-7.15 (m, 1H), 7.04 (d, J=7.2 Hz, 1H), 6.71 (d, J=8.4 Hz, 1H), 5.18 (s, 2H), 4.98-4.86 (m, 1H), 3.93 (s, 3H), 3.84 (s, 2H), 2.81-2.68 (m, 2H), 2.31 (br t, J=9.6 Hz, 2H), 1.98-1.86 (m, 2H), 1.69-1.54 (m, 2H)

### Example A123—Preparation of Compound 197. 2-(4-(6-((4-Chloro-2-fluorobenzyl)oxy)pyridin-2-yl)-2,5-difluorobenzyl)-4-(difluoromethoxy)-1-methyl-1H-benzo[d]imidazole-6-carboxylic acid (Compound 197) (7)

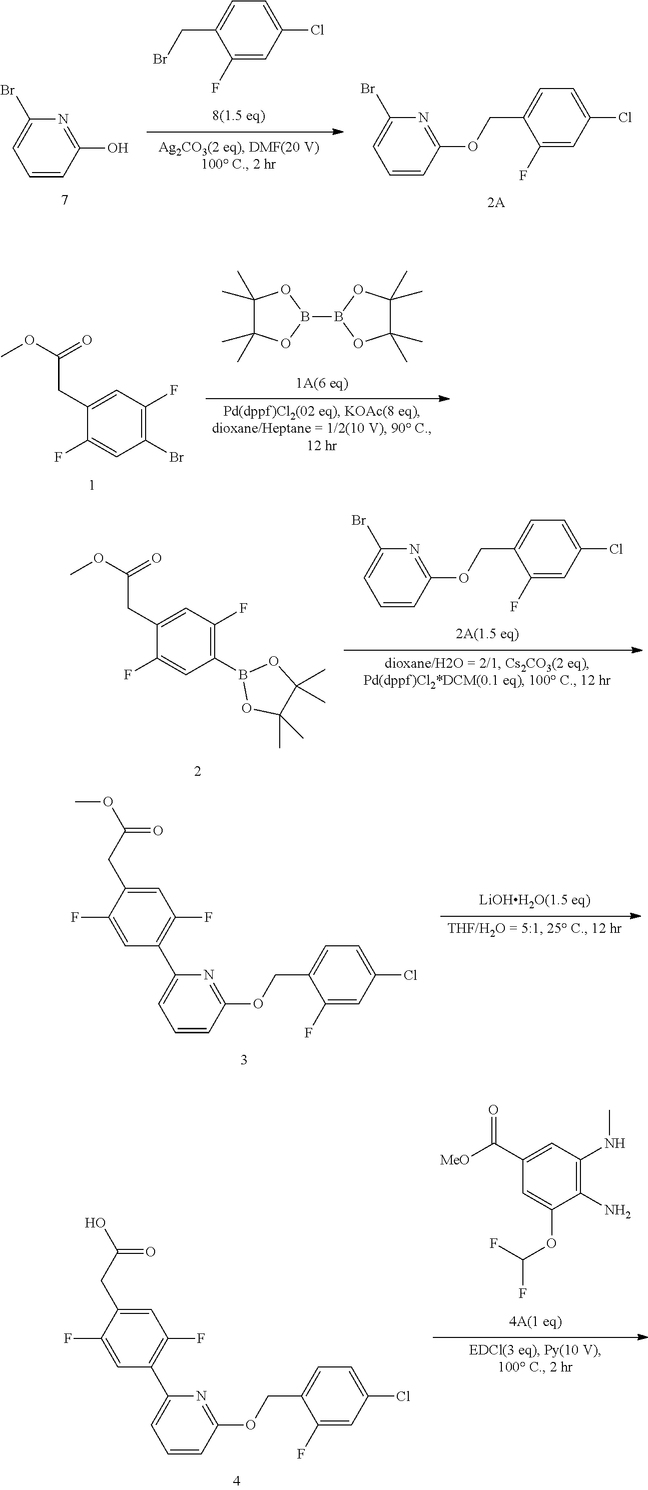

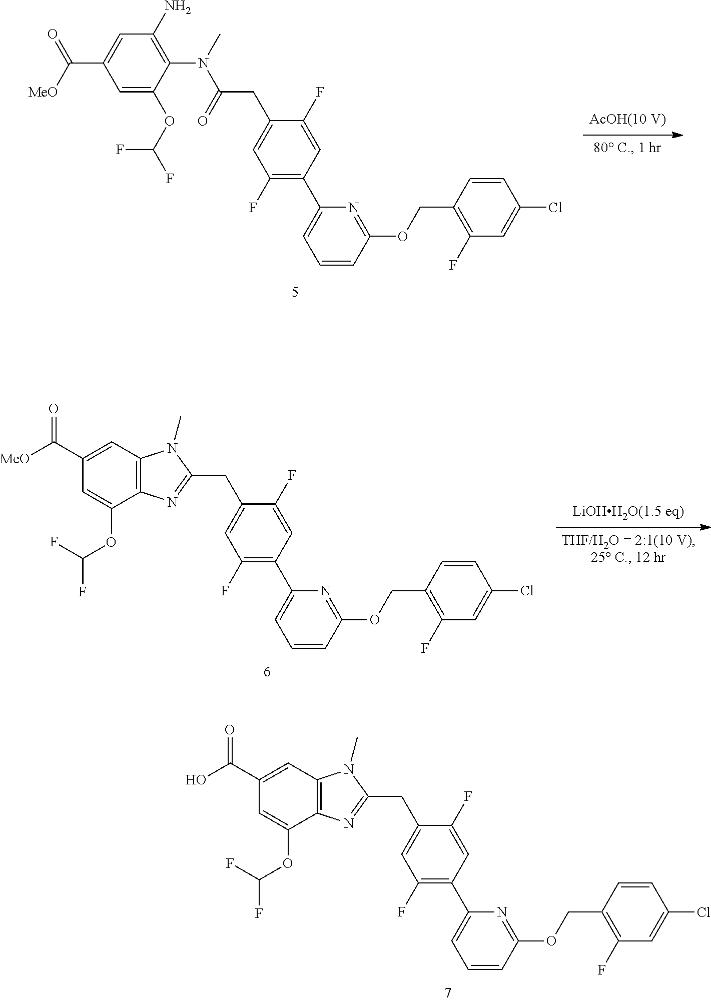

### General Procedure for Preparation of Intermediate 2A:

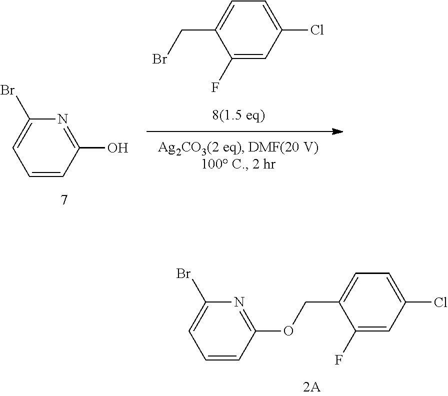 [SMILES: Fc1cc(Cl)ccc1CBr ; Oc1cccc(Br)n1 ; Fc1cc(Cl)ccc1COc1cccc(Br)n1]

To a solution of 7 (5 g, 28.74 mmol, 1 eq) in DMF (100 mL) was added Ag₂CO₃ (15.85 g, 57.47 mmol, 2.61 mL, 2 eq) and 8 (9.63 g, 43.10 mmol, 1.5 eq). The mixture was stirred at 100° C. for 2 hr. TLC (Petroleum ether/Ethyl acetate=10/1, Rf=0.4) indicated 7 was consumed completely and one new spot formed. The reaction was clean according to TLC. The suspension was filtered, and the filter cake was washed with EtOAc (200 mL). The combined filtrates were diluted with H₂O (100 mL) and extracted with EtOAc (100 mL*3). The combined organic layers were washed with H₂O (50 mL*6), dried over Na₂SO₄, filtered, and concentrated under reduced pressure to give a residue. The residue was purified by column chromatography (SiO₂, Petroleum ether/Ethyl acetate=1/0 to 3/1). 2A (13.07 g, 40.88 mmol, 71.12% yield) was obtained as a white solid.

LCMS: RT=1.572 min, MS cal.: 316.6, [M+H]⁺=317.9

¹HNMR (400 MHz, CHCl3-d) δ=7.46 (td, J=8.0, 10.4 Hz, 2H), 7.18-7.08 (m, 3H), 6.74 (d, J=8.4 Hz, 1H), 5.39 (s, 2H)

### General Procedure for Preparation of Intermediate 2:

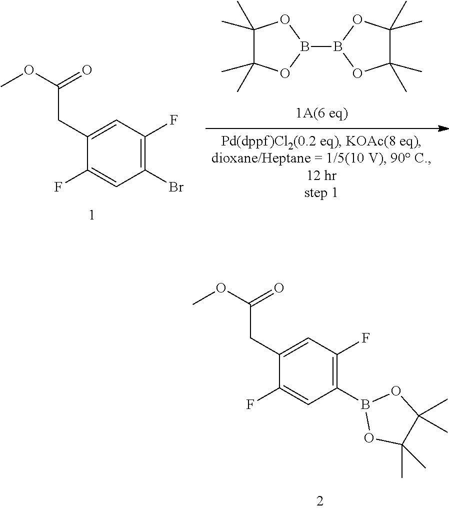

To a solution of 1 (1.5 g, 5.66 mmol, 1 eq) in dioxane (5 mL) and Heptane (10 mL) was added KOAc (4.44 g, 45.27 mmol, 8 eq) and 1A (8.62 g, 33.96 mmol, 6 eq), Pd(dppf)Cl₂ (828.19 mg, 1.13 mmol, 0.2 eq) at 25° C. The mixture was stirred at 90° C. for 12 hrs. TLC showed 1 was consumed completely. The reaction mixture was filtered. The residue was diluted with H₂O (30 mL) and extracted with EtOAc (20 mL). The combined organic layers were washed with brine (20 mL), dried over Na₂SO₄, filtered, and concentrated under reduced pressure to give a residue. The residue was purified by column chromatography (SiO₂, Petroleum ether/Ethyl acetate=1/0 to 0/1, Rf=0.32). 2 (5 g, 8.01 mmol, 70.77% yield, 50% purity) was obtained as a white solid.

¹H NMR (400 MHz, CHCl3-d) δ=7.37 (dd, J=4.8, 9.2 Hz, 1H), 6.94 (dd, J=5.6, 8.7 Hz, 1H), 3.69 (s, 3H), 3.64 (s, 2H), 1.33 (s, 12H), 1.24 (s, 56H)

### General Procedure for Preparation of Intermediate 3:

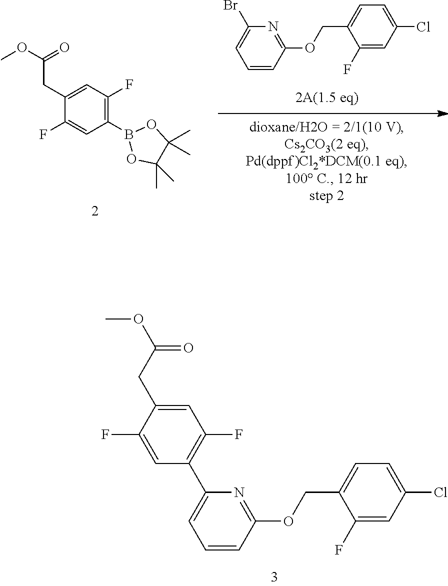

To a solution of 2A (760.66 mg, 1.20 mmol, 1.5 eq) and 2 (1 g, 800.98 μmol, 1 eq, purity 50%) in dioxane (3.5 mL) and H₂O (1.5 mL) was added Cs₂CO₃ (521.95 mg, 1.60 mmol, 2 eq) and Pd(dppf)Cl₂*DCM (65.41 mg, 80.10 μmol, 0.1 eq) at 25° C. The mixture was stirred at 100° C. for 12 hr. TLC showed 2 was consumed completely. The reaction mixture was filtered. The residue was diluted with H₂O (20 mL) and extracted with EtOAc (15 mL). The combined organic layers were washed with brine (20 mL), dried over Na₂SO₄, filtered and concentrated under reduced pressure to give a residue. The residue was purified by column chromatography (SiO₂, Petroleum ether/Ethyl acetate=1/0 to 0/1, Rf=0.36). 3 (300 mg, 711.24 μmol, 44.40% yield) was obtained as a yellow oil.

LCMS: RT=0.673 min, MS cal.: 417.07, [M+H]⁺=422.0

¹H NMR (400 MHz, CHCl3-d) δ=7.81 (dd, J=6.4, 10.6 Hz, 1H), 7.68 (t, J=8.0 Hz, 1H), 7.54-7.44 (m, 2H), 7.17-7.13 (m, 2H), 7.10 (dd, J=5.6, 11.2 Hz, 1H), 6.80 (d, J=8.4 Hz, 1H), 5.50 (s, 2H), 3.76 (s, 3H), 3.72 (s, 2H)

### General Procedure for Preparation of Intermediate 4:

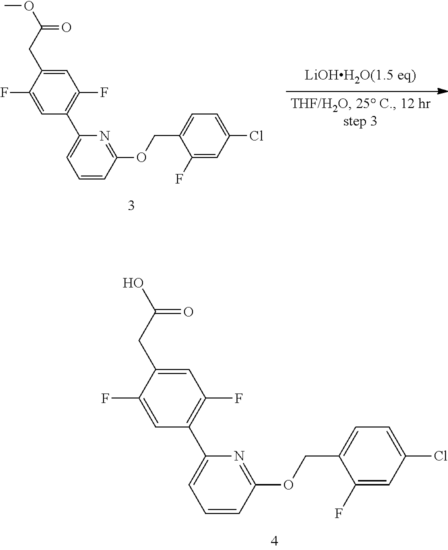 [SMILES: COC(=O)Cc1cc(F)c(-c2cccc(OCc3ccc(Cl)cc3F)n2)cc1F ; "THF/H2O, 25° C., 12 hrstep 3" ; O=C(O)Cc1cc(F)c(-c2cccc(OCc3ccc(Cl)cc3F)n2)cc1F]

To a solution of 3 (300 mg, 711.24 μmol, 1 eq) in THF (2.5 mL) was added LiOH·H₂O (44.77 mg, 1.07 mmol, 1.5 eq) and H₂O (0.5 mL) at 25° C. The mixture was stirred at 25° C. for 12 hr. LC-MS showed 3 was consumed completely. The reaction mixture was added with 0.5 M citric acid to adjust pH=7. Then mixture was diluted with H₂O 15 (mL) and extracted with EtOAc 10 mL. The combined organic layers were washed with brine (15 mL), dried over Na₂SO₄, filtered, and concentrated under reduced pressure to give 4 (200 mg, 490.47 μmol, 68.96% yield) as a white solid.

LCMS: RT=0.612 min, MS cal.: 407.05, [M+H]⁺=408.0

¹H NMR (400 MHz, CHCl3-d) δ=7.82 (dd, J=6.4, 10.6 Hz, 1H), 7.68 (t, J=7.6 Hz, 1H), 7.54-7.44 (m, 2H), 7.17-7.13 (m, 2H), 7.13-7.08 (m, 1H), 6.80 (d, J=8.4 Hz, 1H), 5.50 (s, 2H), 3.77 (s, 2H)

### General Procedure for Preparation of Intermediate 6&5:

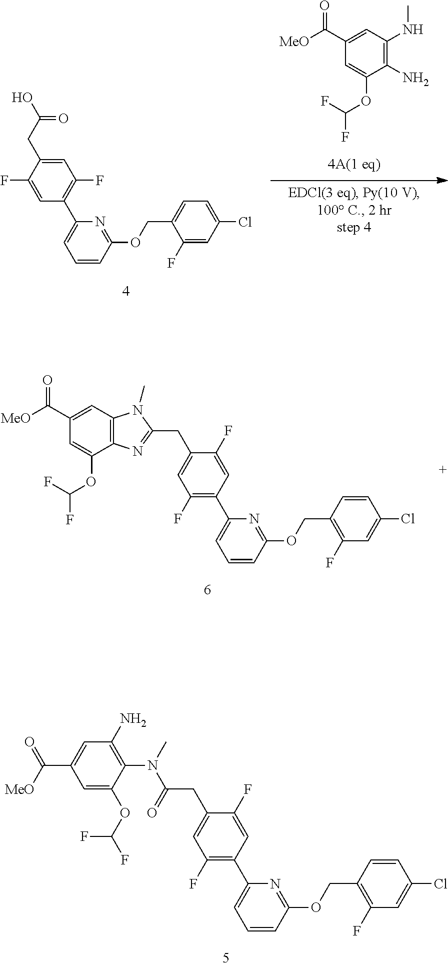 [SMILES: O=C(O)Cc1cc(F)c(-c2cccc(OCc3ccc(Cl)cc3F)n2)cc1F ; CNc1cc(C(=O)OC)cc(OC(F)F)c1N ; "EDCl(3 eq), Py(10 V),100° C., 2 hrstep 4" ; COC(=O)c1cc(OC(F)F)c2nc(Cc3cc(F)c(-c4cccc(OCc5ccc(Cl)cc5F)n4)cc3F)n(C)c2c1 ; COC(=O)c1cc(N)c(N(C)C(=O)Cc2cc(F)c(-c3cccc(OCc4ccc(Cl)cc4F)n3)cc2F)c(OC(F)F)c1]

To a solution of 4 (300 mg, 735.71 μmol, 1 eq) in Py (3 mL) was added 4A (181.14 mg, 735.71 μmol, 1 eq) and EDCI (423.11 mg, 2.21 mmol, 3 eq) at 25° C. The mixture was stirred at 100° C. for 2 hr. TLC showed 4 was consumed completely. The reaction mixture was diluted with H₂O (20 mL) and extracted with EtOAc (15 mL). The combined organic layers were washed with brine (20 mL), dried over Na₂SO₄, filtered and concentrated under reduced pressure to give a residue. 6 (20 mg, 32.37 μmol, 4.40% yield) was obtained as a white solid. 5 (200 mg, 314.48 μmol, 42.75% yield) was obtained as a white solid.

### General Procedure for Preparation of Intermediate 6:

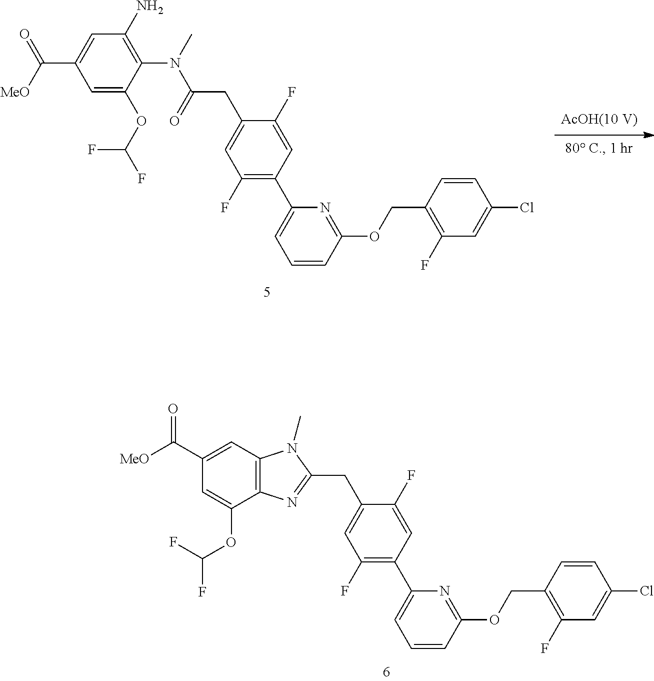 [SMILES: COC(=O)c1cc(N)c(N(C)C(=O)Cc2cc(F)c(-c3cccc(OCc4ccc(Cl)cc4F)n3)cc2F)c(OC(F)F)c1 ; "80° C., 1 hr" ; COC(=O)c1cc(OC(F)F)c2nc(Cc3cc(F)c(-c4cccc(OCc5ccc(Cl)cc5F)n4)cc3F)n(C)c2c1]

To a solution of 5 (200 mg, 314.48 μmol, 1 eq) in CH₃COOH (2 mL). The mixture was stirred at 80° C. for 1 hr. TLC showed 5 was consumed completely. The reaction mixture was concentrated under reduced pressure to remove CH₃COOH. The reaction mixture was diluted with NaHCO₃ (0.5 mL) and extracted with EtOAc (6 mL). The combined organic layers were washed with brine (5 mL) dried over Na₂SO₄, filtered, and concentrated under reduced pressure to give a residue. 6 (150 mg, 242.74 μmol, 77.19% yield) was obtained as a white solid.

LCMS: RT=0.682 min, MS cal.: 617.11, [M+H]⁺=618.1.

### General Procedure for Preparation of Compound 197 (7):

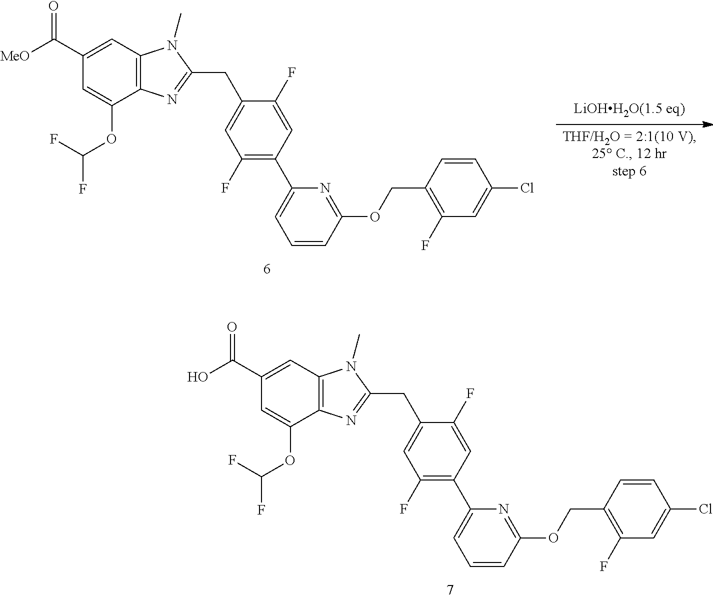

To a solution of 6 (150 mg, 242.74 μmol, 1 eq) in THF (1 mL) was added LiOH·H₂O (15.28 mg, 364.11 μmol, 1.5 eq) and H₂O (0.5 mL) at 25° C. The mixture was stirred at 25° C. for 12 hr. TLC showed 6 was consumed completely. The reaction mixture was filtered to give a residue. The residue was purified by prep-HPLC (column: Waters Xbridge Prep OBD C18 150*40 mm*10 um; mobile phase: [H₂O (10 mM NH₄HCO₃)-ACN]; gradient: 30%-50% B over 8.0 min). 2-(4-(6-((4-Chloro-2-fluorobenzyl)oxy)pyridin-2-yl)-2,5-difluorobenzyl)-4-(difluoromethoxy)-1-methyl-1H-benzo[d]imidazole-6-carboxylic acid (12.03 mg, 19.92 μmol, 8.21% yield) was obtained as a white solid.

LCMS: RT=3.347 min, MS cal.: 603.10, [M+H]⁺=604.0.

HPLC: RT=9.871 min, purity: 97.27%

¹H NMR (400 MHz, CHCl3-d) δ=8.02 (s, 1H), 7.90-7.81 (m, 2H), 7.75 (s, 1H), 7.61 (t, J=8.0 Hz, 1H), 7.54-7.47 (m, 3H), 7.37 (t, J=74.4 Hz, 1H) 7.30 (m, 2H), 6.94 (d, J=8.4 Hz, 1H), 5.50 (s, 2H), 4.44 (s, 2H), 3.89 (s, 3H)

### Example A124—Preparation of Compound 198. 2-(4-(6-((4-Chloro-2-fluorobenzyl)oxy)pyridin-2-yl)-2-fluorobenzyl)-4-(difluoromethoxy)-1-methyl-1H-benzo[d]imidazole-6-carboxylic acid (Compound 198) (5)

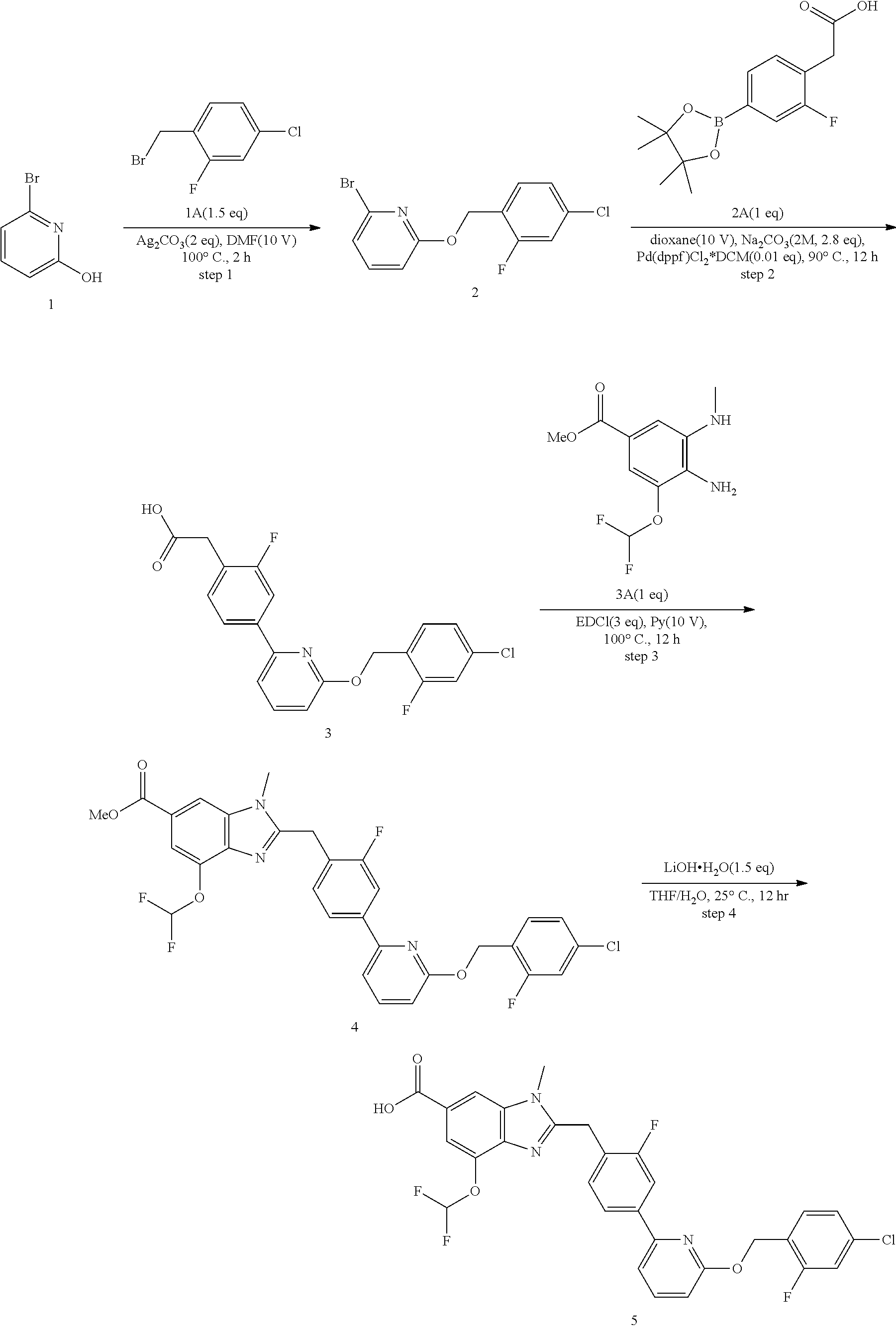

### General Procedure for Preparation of Intermediate 2:

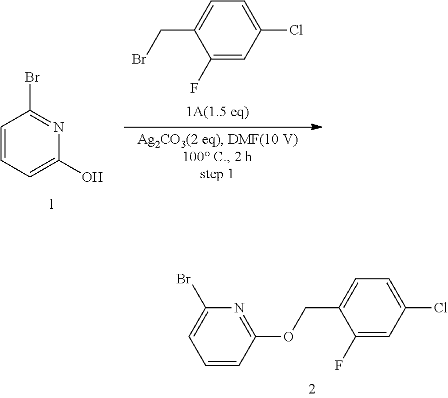

A mixture of 1 (10 g, 57.47 mmol, 1 eq), 1A (19.27 g, 86.21 mmol, 1.5 eq), Ag₂CO₃ (31.70 g, 114.95 mmol, 5.22 mL, 2 eq), in DMF (100 mL) was degassed and purged with N₂ for 3 times, and then the mixture was stirred at 100° C. for 2 hr under N₂ atmosphere. LCMS showed 1 was consumed completely and one main peak with desired mass was detected. The suspension was filtered through diatomite and filter cake was washed with EtOAc 30 ml*3. The combined filtrate were diluted with H₂O 20 mL and extracted with EtOAc 60 mL (20 mL*3). The combined organic layers were washed with brine 20 mL, dried over Na₂SO₄, filtered, and concentrated under reduced pressure to give a residue. The residue was purified by column chromatography (SiO₂, Petroleum ether/Ethyl acetate=100/1 to 10/1). 2 (18 g, 56.86 mmol, 98.94% yield) was obtained as a white solid.

LCMS: RT=0.662 MS cal.: 314.9, [M+H]⁺=315.9

1H NMR (400 MHz, DMSO-d6) δ=7.71 7.66m, 1H) 7.59 (t, J=8.0 Hz, 1H) 7.48 (dd, J=10.00, 2.04 Hz, 1H) 7.33 (dd, J=8.4, 1.73 Hz, 1H) 7.26 (d, J=7.2 Hz, 1H) 6.93 (d, J=8.0 Hz, 1H) 5.34 (s, 2H)

### General Procedure for Preparation of Intermediate 3:

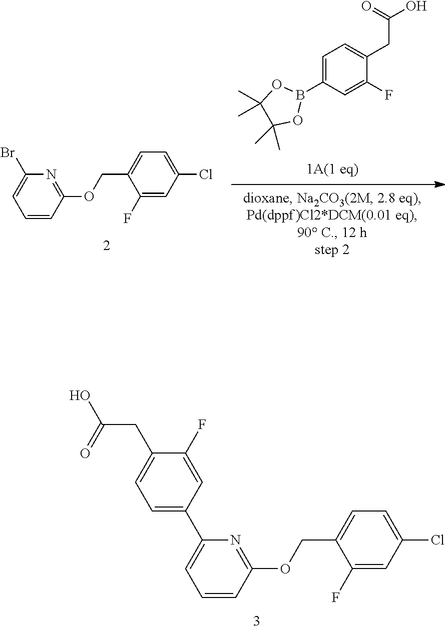 [SMILES: CC1(C)OB(c2ccc(CC(=O)O)c(F)c2)OC1(C)C ; Fc1cc(Cl)ccc1COc1cccc(Br)n1 ; "1A(1 eq)" ; "dioxane, Na2CO3(2M, 2.8 eq),Pd(dppf)Cl2*DCM(0.01 eq),90° C., 12 hstep 2" ; O=C(O)Cc1ccc(-c2cccc(OCc3ccc(Cl)cc3F)n2)cc1F]

A mixture of 2 (700 mg, 2.21 mmol, 1 eq), 2A (743.27 mg, 2.65 mmol, 1.2 eq), Na₂CO₃ (2 M, 3.10 mL, 2.8 eq) in dioxane (7 mL) was sparged with N₂ for 5 min, and Pd(dppf)Cl₂·CH₂Cl₂ (18.06 mg, 22.11 μmol, 0.01 eq) was then added to the mixture. The mixture was degassed and purged with N₂ for 3 times, and then the mixture was stirred at 90° C. for 12 hr under N₂ atmosphere. LCMS indicated 2 was consumed completely and one main peak with desired mass was detected. The residue was poured into water (15 mL). The aqueous phase was extracted with EtOAc (15 mL*3). The combined organic phase was washed with brine (15 mL*2), dried with anhydrous Na₂SO₄, filtered, and concentrated in vacuum. The residue was purified by column chromatography (SiO₂, Petroleum ether/Ethyl acetate=10/1 to 3/1). 3 (800 mg, 2.05 mmol, 92.82% yield) was a brown solid.

LCMS: RT=2.136 min, MS cal.: 389.1, [M+H]⁺=390.1

¹H NMR (400 MHz, CHCl3-d) δ=7.76-7.64 (m, 4H), 7.51-7.32 (m, 4H), 6.82 (d, J=8.0 Hz, 1H), 5.62 (s, 2H), 3.78 (s, 2H), 1.28 (s, 1H)

### General Procedure for Preparation of Intermediate 4:

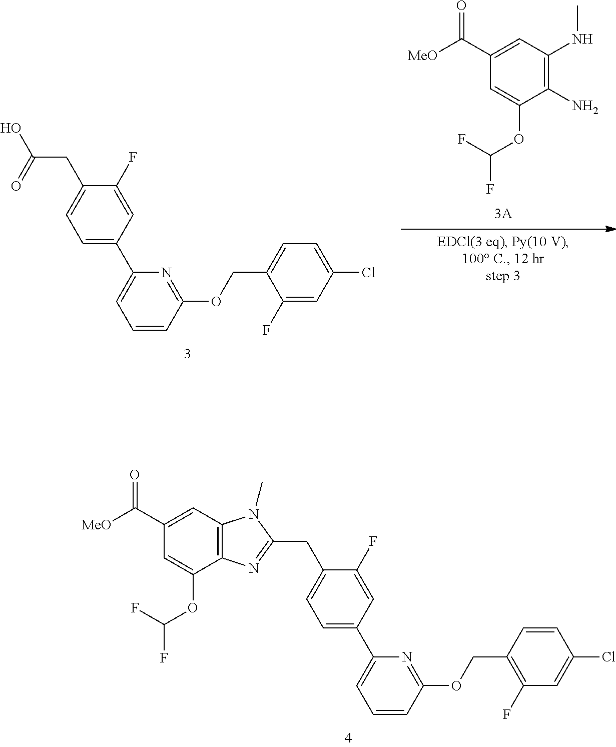

To a solution of 3A (Synthesized from Int 3) (100 mg, 406.16 μmol, 1 eq) in Py (2 mL) was added EDCI (233.58 mg, 1.22 mmol, 3 eq) and 3 (158.31 mg, 406.16 μmol, 1 eq). The mixture was stirred at 100° C. for 12 hr. LCMS indicated 3 was consumed completely and one main peak with desired mass was detected. The residue was poured into water (10 mL). The aqueous phase was extracted with ethyl acetate (10 mL*3). The combined organic phase was washed with brine (10 mL*2), dried with anhydrous Na₂SO₄, filtered and concentrated in vacuum. The residue was purified by prep-HPLC (column: Waters Xbridge Prep OBD C18 150*40 mm*10 um; mobile phase: [H₂O (10 mM NH₄HCO₃)-ACN]; gradient: 40%-60% B over 8.0 min). 4 (120 mg, crude) was a pink solid.

LCMS: RT=1.761 min, MS cal.: 599.1, [M+H]⁺=600.0

### General Procedure for Preparation of Compound 198 (5):

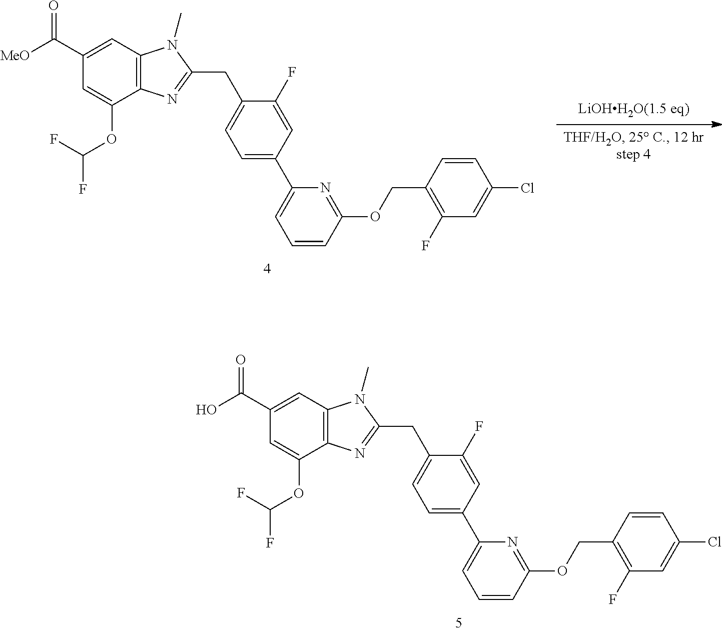 [SMILES: COC(=O)c1cc(OC(F)F)c2nc(Cc3ccc(-c4cccc(OCc5ccc(Cl)cc5F)n4)cc3F)n(C)c2c1 ; "LiOH·H2O(1.5 eq)" ; "THF/H2O, 25° C., 12 hrstep 4" ; Cn1c(Cc2ccc(-c3cccc(OCc4ccc(Cl)cc4F)n3)cc2F)nc2c(OC(F)F)cc(C(=O)O)cc21]

To a solution of 4 (90 mg, 150.01 μmol, 1 eq) in THF (0.7 mL) and H₂O (0.3 mL) was added LiOH·H₂O (9.44 mg, 225.02 μmol, 1.5 eq). The mixture was stirred at 25° C. for 12 hr. LCMS showed 4 was consumed completely and one main peak with desired mass was detected. The reaction mixture was filtered and concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 30%-50% B over 8.0 min). 2-(4-(6-((4-Chloro-2-fluorobenzyl)oxy)pyridin-2-yl)-2-fluorobenzyl)-4-(difluoromethoxy)-1-methyl-1H-benzo[d]imidazole-6-carboxylic acid (24.15 mg, 40.33 μmol, 26.89% yield, 97% purity) was obtained as a white solid.

LCMS: RT=2.920 min, MS cal.: 585.1, [M+H]⁺=586.1

HPLC: RT=16.390 min, purity: 97.86%

¹H NMR (400 MHz, MeOH-d4) δ=8.00 (s, 1H), 7.89-7.84 (m, 1H), 7.81 (dd, J=1.6, 8.0 Hz, 1H), 7.77-7.72 (m, 1H), 7.70 (s, 1H), 7.56-7.51 (m, 1H), 7.50 (s, 1H), 7.25-7.20 (m, 3H), 7.18 (t, J=74.4 Hz, 1H), 6.81 (s, 1H), 5.53 (s, 2H), 4.46 (s, 2H), 3.81 (s, 3H)

### Example A125—Preparation of Compound 199. 2-((4-(6-((4-Chloro-2-fluorobenzyl)oxy)pyridin-2-yl)piperidin-1-yl)methyl)-1-cyclopropyl-4-(difluoromethoxy)-1H-benzo[d]imidazole-6-carboxylic acid (Compound 199) (6)

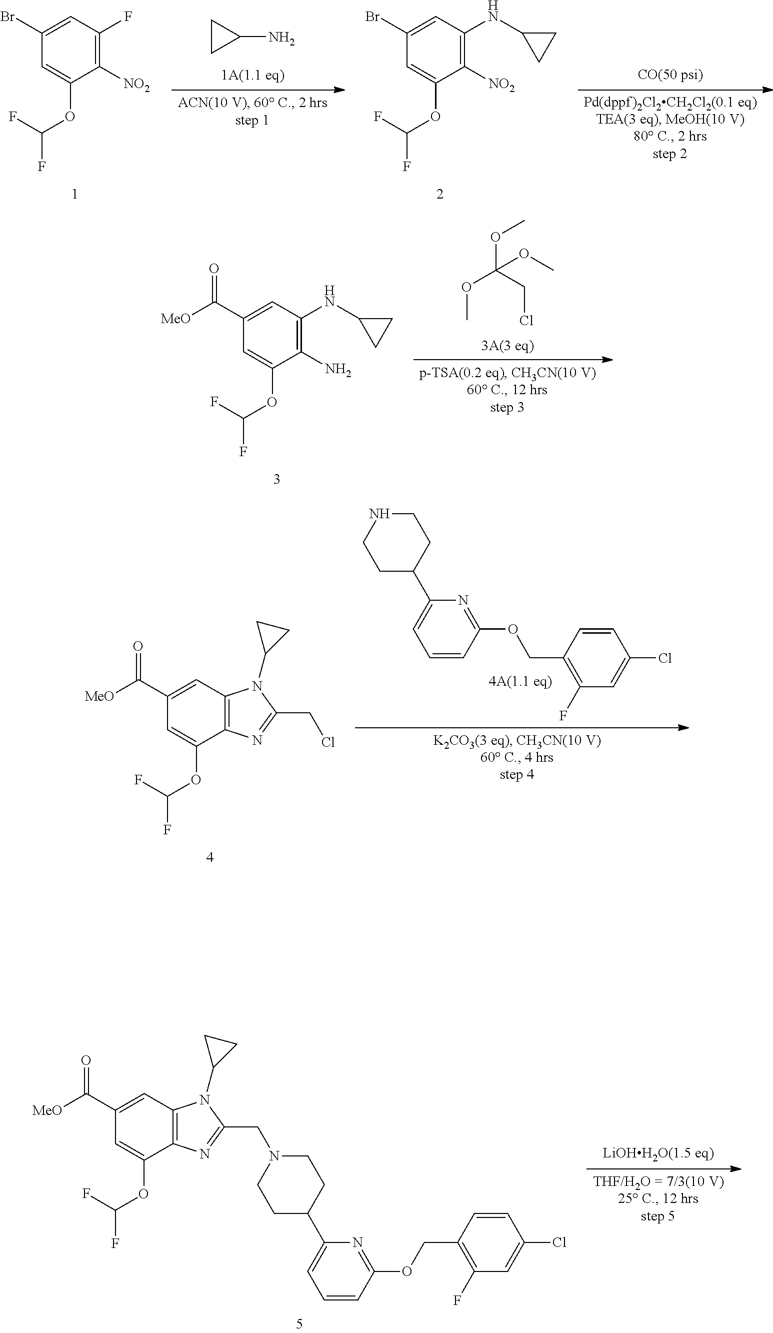

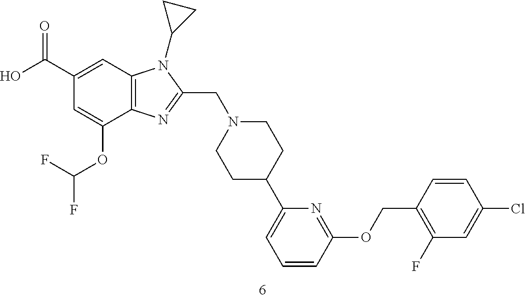 [SMILES: O=C(O)c1cc(OC(F)F)c2nc(CN3CCC(c4cccc(OCc5ccc(Cl)cc5F)n4)CC3)n(C3CC3)c2c1]

### General Procedure for Preparation of Intermediate 2:

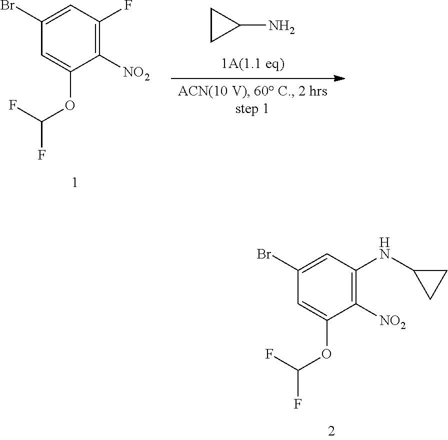

To a mixture of 1 (1.8 g, 6.29 mmol, 1 eq) and 1A (395.26 mg, 6.92 mmol, 479.69 μL, 1.1 eq) in ACN (18 mL) was stirred at 60° C. for 2 hours. The color changes to yellow. TLC (SiO₂, PE:EA=3:1, R_{f}=0.60) showed the reaction was completed. The reaction mixture was quenched by addition water (10 mL), and then extracted with EA (10 mL*3). The combined organic layers were washed with brine (30 mL), dried over Na₂SO₄, filtered, and concentrated under reduced pressure to give a residue. The residue was purified by column chromatography (SiO₂, Petroleum ether/Ethyl acetate=10/1 to 8/1). 2 (1.6 g, 4.95 mmol, 78.69% yield)) was obtained as a yellow solid.

¹H NMR (400 MHz, CHCl3-d) δ=7.30 (d, J=2.0 Hz, 1H), 6.76 (s, 1H), 6.58 (s, 1H), 6.53 (t, J=72.8 Hz, 1H), 2.55-2.48 (m, 1H), 0.95-0.88 (m, 2H), 0.66-0.59 (m, 2H)

### General Procedure for Preparation of Intermediate 3:

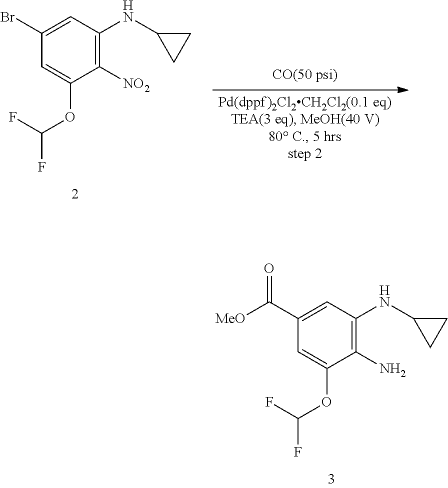 [SMILES: O=[N+]([O-])c1c(NC2CC2)cc(Br)cc1OC(F)F ; "CO(50 psi)" ; "Pd(dppf)2Cl2·CH2Cl2(0.1 eq)TEA(3 eq), MeOH(40 V)80° C., 5 hrsstep 2" ; COC(=O)c1cc(NC2CC2)c(N)c(OC(F)F)c1]

MeOH (28 mL) and TEA (657.71 mg, 6.50 mmol, 904.69 μL, 3 eq) was charged to the 75 mL Hydrogenated bottle, then 2 (700 mg, 2.17 mmol, 1 eq) and Pd(dppf)Cl₂·CH₂Cl₂ (176.93 mg, 216.66 μmol, 0.1 eq) was added at 25° C. After the addition, the mixture was degassed and purged with CO for 3 times. The mixture was stirred at 80° C. (50 psi) for 5 hrs. The color changes to orange. LCMS showed the reaction was completed. The mixture was filtered and concentrated in vacuum. The mixture was used for the next step directly without purification. 3 (1.36 g, crude) was obtained as orange oil.

LCMS: Rt=1.156 min, MS cal.: 272.1, [M+H]⁺=272.9

### General Procedure for Preparation of Intermediate 4:

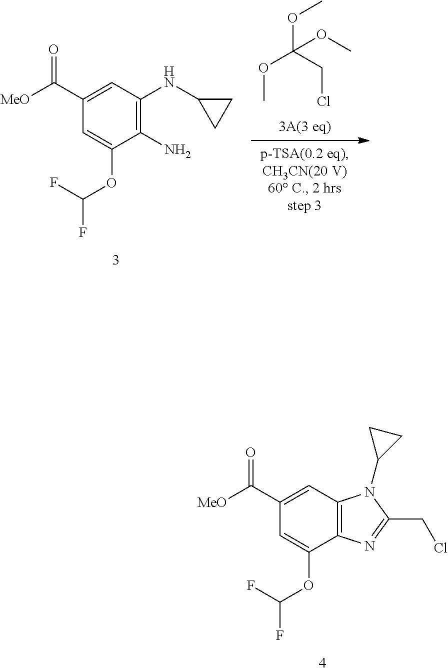

To a mixture of 3 (1 g, 3.67 mmol, 1 eq) in ACN (20 mL) was added 3A (1.7 g, 11.00 mmol, 1.49 mL, 3 eq) and p-TsOH (126.52 mg, 734.64 μmol, 0.2 eq). The mixture was stirred at 60° C. for 2 hours. The color changes to brown. TLC (SiO₂, PE:EA=2:1, Rf=0.50) showed the reaction was completed. The mixture was concentrated in vacuum. The residue was purified by prep-TLC (SiO₂, PE:EA=2:1, R_{f}=0.50). 4 (209 mg, 586.40 μmol, 15.96% yield) was obtained as yellow oil.

LCMS: Rt=0.489 min, purity: 92.79%

¹H NMR (400 MHz, MeOH-d4) δ=8.23-8.20 (m, 1H), 7.68 (s, 1H), 7.29 (t, J=74.0 Hz, 1H), 5.03 (s, 2H), 3.96 (s, 3H), 3.55-3.46 (m, 1H), 1.42-1.35 (m, 2H), 1.29-1.25 (m, 2H)

### General Procedure for Preparation of Intermediate 5:

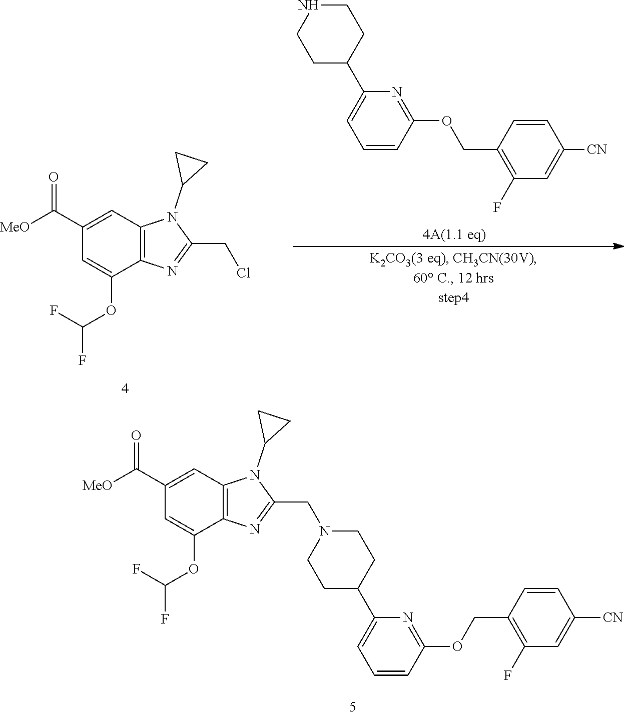

To a mixture of 4 (100 mg, 302.38 μmol, 1 eq) and 4A (106.70 mg, 332.61 μmol, 1.1 eq) in ACN (3 mL) was added K₂CO₃ (125.37 mg, 907.13 μmol, 3 eq). The mixture was stirred at 60° C. for 12 hours. The color changes to brown. TLC (SiO₂, PE:EA=1:1, R_{f}=0.50) showed the reaction was completed. The mixture was filtered and concentrated in vacuum. The residue was purified by prep-TLC (SiO₂, PE:EA=1:1, R_{f}=0.45). 5 (140 mg, 223.83 μmol, 74.02% yield) was obtained as light-yellow oil.

HPLC: Rt=3.199 min, purity: 98.33%

### General Procedure for Preparation of Compound 199 (6):

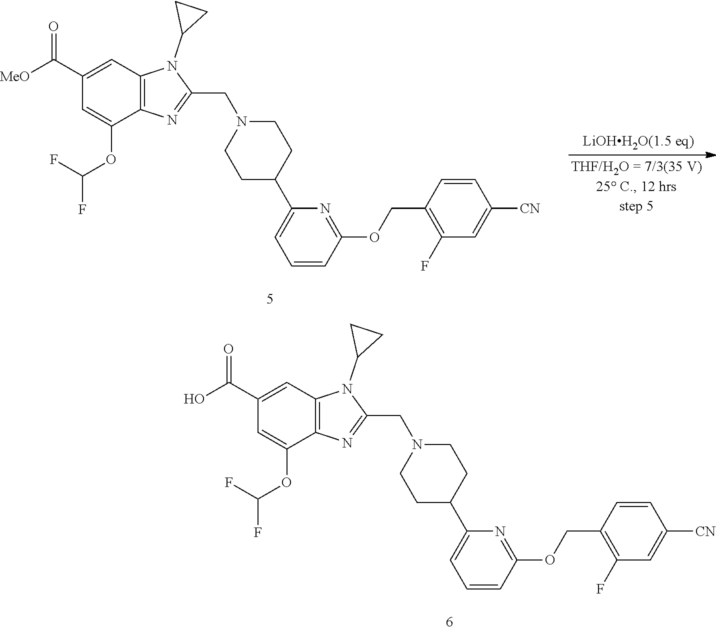 [SMILES: COC(=O)c1cc(OC(F)F)c2nc(CN3CCC(c4cccc(OCc5ccc(C#N)cc5F)n4)CC3)n(C3CC3)c2c1 ; N#Cc1ccc(COc2cccc(C3CCN(Cc4nc5c(OC(F)F)cc(C(=O)O)cc5n4C4CC4)CC3)n2)c(F)c1]

To a mixture of 5 (140 mg, 227.63 μmol, 1 eq) and LiOH. H₂O (14.33 mg, 341.44 μmol, 1.5 eq) in THF (3.5 mL) and H₂O (1.5 mL) was stirred at 25° C. for 12 hours. The color changes to yellow. LCMS showed 79% of desired product was detected. The mixture was concentrated in vacuum. The residue was purified by prep-HPLC (column: Waters Xbridge Prep OBD C18 150*40 mm*10 um; mobile phase: [H₂O (10 mM NH₄HCO₃)-ACN]; gradient: 15%-65% B over 8.0 min). 2-((4-(6-((4-Chloro-2-fluorobenzyl)oxy)pyridin-2-yl)piperidin-1-yl)methyl)-1-cyclopropyl-4-(difluoromethoxy)-1H-benzo[d]imidazole-6-carboxylic acid (29.31 mg, 48.74 μmol, 21.41% yield) was obtained as a white solid.

LCMS: Rt=2.878 min, MS cal.: 600.2, [M+H]⁺=601.2.

HPLC: Rt=12.914 min, purity: 99.95%

¹H NMR (400 MHz, DMSO-d6) δ=8.06 (s, 1H), 7.62 (t, J=72.0 Hz, 1H), 7.61-7.59 (m, 1H), 7.57-7.52 (m, 2H), 7.45-7.40 (dd, J=2.0, 10.4 Hz, 1H), 7.27 (dd, J=1.6, 8.4 Hz, 1H), 6.86 (d, J=7.6 Hz, 1H), 6.66 (d, J=8.0 Hz, 1H), 5.35 (s, 2H), 3.87 (s, 2H), 3.51-3.45 (m, 1H), 3.02 (br d, J=11.2 Hz, 2H), 2.64-2.54 (m, 1H), 2.28 (br t, J=10.0 Hz, 2H), 1.84-1.76 (m, 2H), 1.75-1.63 (m, 2H), 1.30-1.23 (m, 2H), 1.23-1.16 (m, 2H)

### Example A126—Preparation of Compound 200. 2-((4-(5-((4-Chloro-2-fluorobenzyl)oxy)-1H-pyrazol-1-yl)piperidin-1-yl)methyl)-4-(difluoromethoxy)-1-methyl-1H-benzo[d]imidazole-6-carboxylic acid (Compound 200) (7)

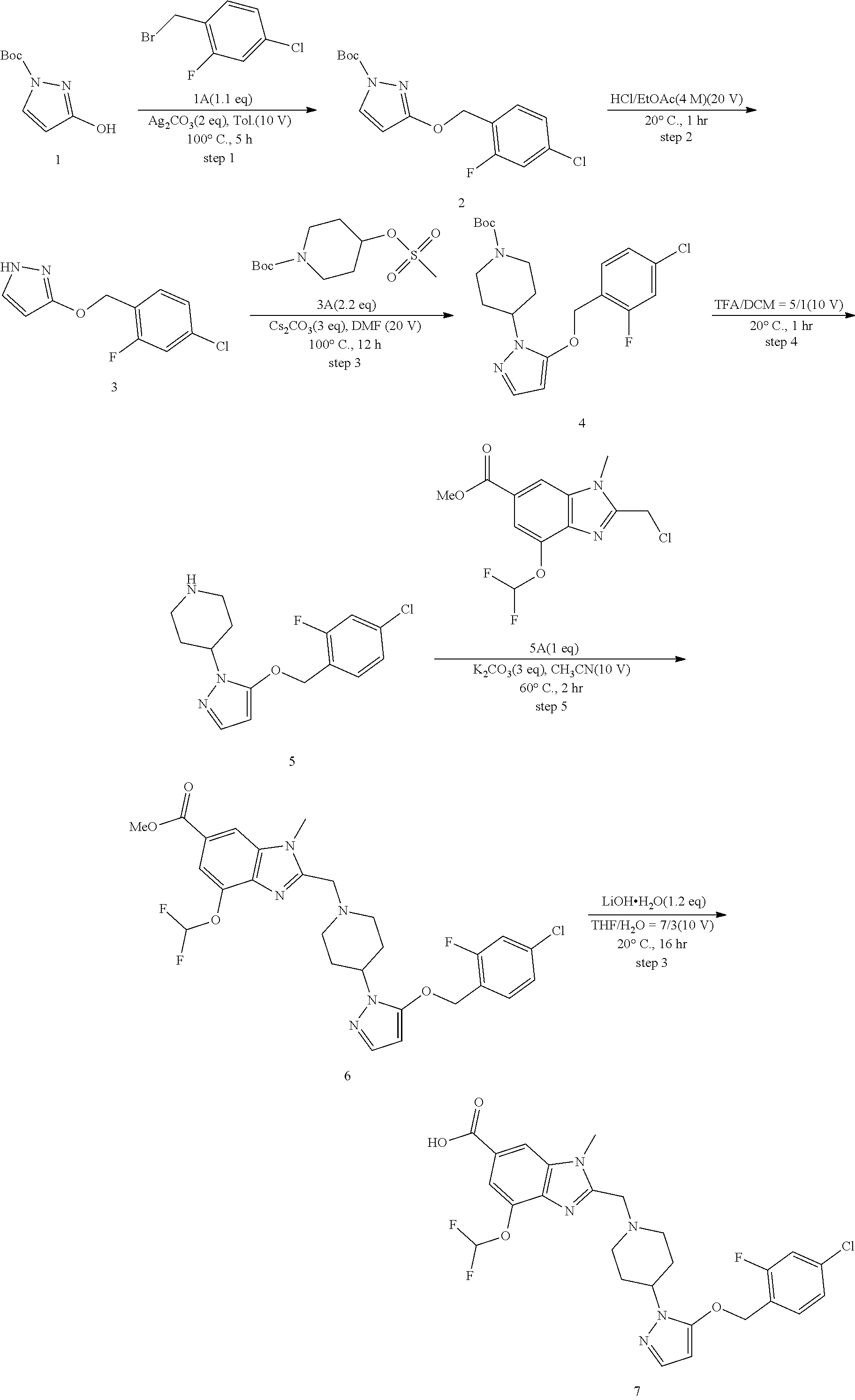

### General Procedure for Preparation of Intermediate 2:

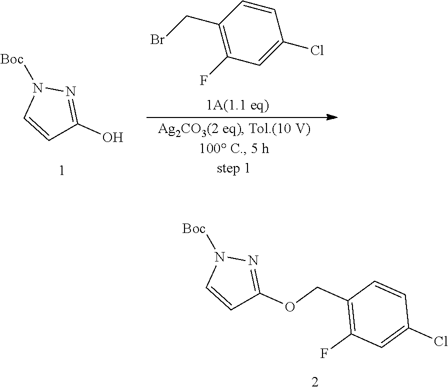 [SMILES: CC(C)(C)OC(=O)n1ccc(O)n1 ; Fc1cc(Cl)ccc1CBr ; CC(C)(C)OC(=O)n1ccc(OCc2ccc(Cl)cc2F)n1]

To a mixture of 1A (2.00 g, 8.96 mmol, 1.1 eq) and 1 (1.5 g, 8.14 mmol, 1 eq) in Toluene (15 mL) was added Ag₂CO₃ (4.49 g, 16.29 mmol, 739.04 μL, 2 eq) in one portion at 20° C. under N₂. The mixture was stirred at 100° C. and stirred for 5 hours. LC-MS (RT=0.899 min) showed 1 was consumed completely and one main peak with desired mass was detected. The mixture was filtered and concentrated in vacuum. The residue was purified by column chromatography (SiO₂, PE/EtOAc=10/1 to 0/1). 2 (1.7 g, 5.20 mmol, 63.89% yield) was obtained as colorless oil.

LCMS: RT=0.899 min, MS cal.: 326.1, [M−(t-Bu)+H]⁺=271.0

¹H NMR (400 MHz, MeOH-d4) δ=8.02 (d, J=3.2 Hz, 1H) 7.56 (t, J=8.0 Hz, 1H) 7.23-7.30 (m, 2H) 6.05 (d, J=3.2 Hz, 1H) 5.32 (s, 2H) 1.64 (s, 9H)

### General Procedure for Preparation of Intermediate 3:

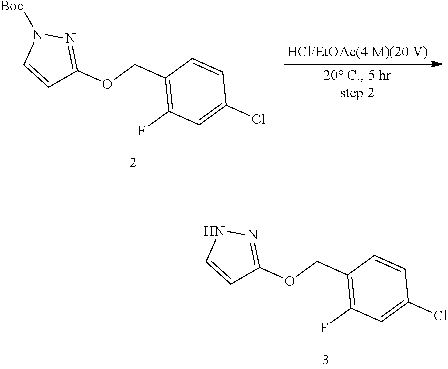 [SMILES: CC(C)(C)OC(=O)n1ccc(OCc2ccc(Cl)cc2F)n1 ; Fc1cc(Cl)ccc1COc1cc[nH]n1]

To a mixture of 2 (1.7 g, 5.20 mmol, 1 eq) in EtOAc (2 mL) was added HCl/EtOAc (4 M, 20 mL, 15.38 eq) at 20° C. The mixture was stirred at 20° C. for 5 hours. LC-MS (product: RT=0.455 min) showed 2 was consumed completely and one main peak with desired mass was detected. The reaction mixture was quenched by saturated NaHCO₃ solution to pH=8. The mixture filtered. The aqueous phase was extracted with DCM (10 mL*3). The combined organic phase was washed with brine (10 mL*3), dried with anhydrous Na₂SO₄, filtered, and concentrated in vacuum. The residue was purified by column chromatography (SiO₂, PE/EtOAc=99/1 to 88/12). 3 (1.02 g, 4.50 mmol, 86.50% yield) was obtained as light-yellow oil.

LCMS: RT=0.455 min, MS cal.: 226.0, [M+H]⁺=227.1

¹H NMR (400 MHz, MeOH-d4) δ=7.51 (t, J=8.4 Hz, 1H) 7.45 (d, J=2.4 Hz, 1H) 7.22-7.25 (m, 1H) 7.20-7.22 (m, 1H) 5.77 (d, J=2.4 Hz, 1H) 5.18 (s, 2H)

### General Procedure for Preparation of Intermediate 4:

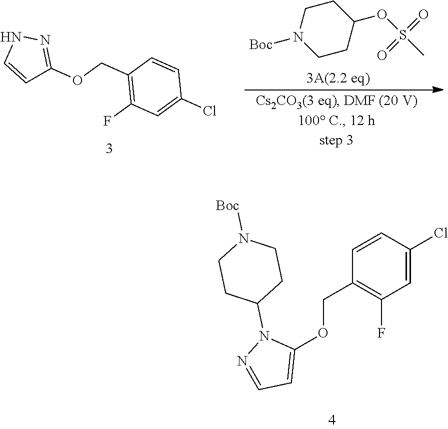 [SMILES: Fc1cc(Cl)ccc1COc1cc[nH]n1 ; CC(C)(C)OC(=O)N1CCC(OS(C)(=O)=O)CC1 ; CC(C)(C)OC(=O)N1CCC(n2nccc2OCc2ccc(Cl)cc2F)CC1]

To a mixture of 3 (300 mg, 1.32 mmol, 1 eq) and 3A (813.52 mg, 2.91 mmol, 2.2 eq) in DMF (6 mL) was added Cs₂CO₃ (1.29 g, 3.97 mmol, 3 eq). The mixture was stirred at 100° C. for 12 hours. LC-MS (product: RT=0.583 min) showed 3 was consumed completely and one main peak with desired mass was detected. The reaction mixture was quenched by addition H₂O (10 mL). The residue was extracted with EtOAc (10 mL*3). The combined organic layers were washed with brine (20 mL*2), dried over Na₂SO₄, filtered and concentrated in vacuum. The residue was purified by column chromatography (SiO₂, PE/EtOAc=99/1 to 92/8). 4 (49 mg, 9.04% yield) was obtained as a light-yellow oil.

LCMS: RT=0.583 min, MS cal.: 409.2, [M−(t-Bu)+H]⁺=354.2

¹H NMR (400 MHz, MeOH-d4) δ=7.52 (t, J=8.0 Hz, 1H) 7.23-7.34 (m, 3H) 5.77 (d, J=2.0 Hz, 1H) 5.20 (s, 2H) 4.32 (tt, J=11.6, 4.0 Hz, 1H) 4.17 (br d, J=13.2 Hz, 2H) 2.72-3.00 (m, 2H) 1.93 (qd, J=12.4, 4.4 Hz, 2H) 1.73-1.85 (m, 2H) 1.46 (s, 9H)

### General Procedure for Preparation of Intermediate 5:

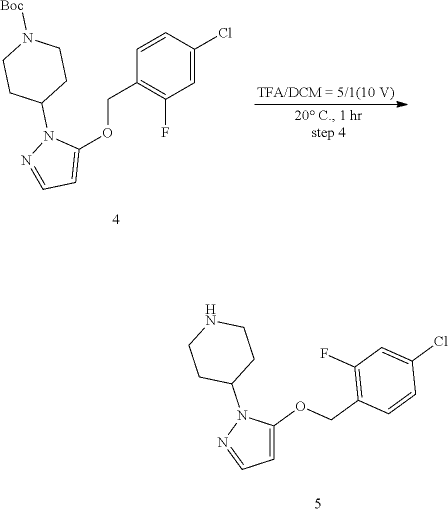 [SMILES: CC(C)(C)OC(=O)N1CCC(n2nccc2OCc2ccc(Cl)cc2F)CC1 ; Fc1cc(Cl)ccc1COc1ccnn1C1CCNCC1]

To a mixture of 4 (39 mg, 95.15 μmol, 1 eq) in DCM (2.5 mL) and TFA (767.50 mg, 6.73 mmol, 0.5 mL, 70.74 eq) was stirred at 20° C. for 1 hour. LC-MS (product: RT=0.379 min) showed 4 was consumed completely and one main peak with desired mass was detected. The mixture was concentrated in vacuum. The mixture was added 10 mL DCM. Then the reaction mixture was quenched by NaHCO₃ solution to pH-8. The aqueous phase was extracted with DCM (10 mL*3). The combined organic phase was washed with brine (10 mL*3), dried with anhydrous Na₂SO₄, filtered, and concentrated in vacuum. The crude product was used into the next step without further purification. 5 (28 mg, 90.39 μmol, 95.00% yield) was obtained as a white solid.

LCMS: RT=0.379 min, MS cal.: 309.1, [M+H]⁺=310.2

¹H NMR (400 MHz, MeOH-d4) δ=7.53 (t, J=8.4 Hz, 1H) 7.24-7.33 (m, 3H) 5.77 (d, J=2.0 Hz, 1H) 5.20 (s, 2H) 4.27 (tt, J=11.6, 4.0 Hz, 1H) 3.18 (br d, J=12.8 Hz, 2H) 2.74 (td, J=12.8, 2.8 Hz, 2H) 1.92-2.10 (m, 2H) 1.83 (br dd, J=12.4, 2.0 Hz, 2H)

### General Procedure for Preparation of Intermediate 6:

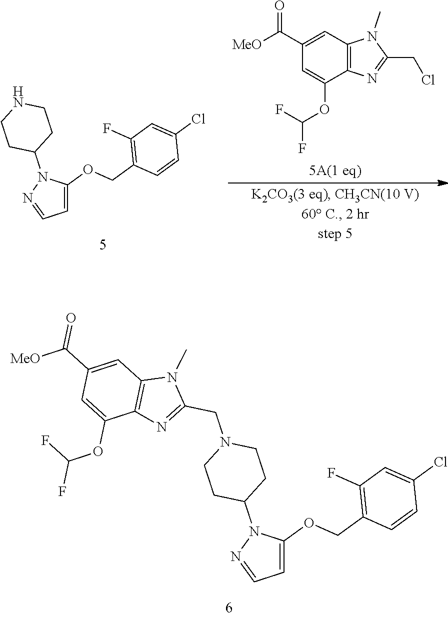 [SMILES: Fc1cc(Cl)ccc1COc1ccnn1C1CCNCC1 ; COC(=O)c1cc(OC(F)F)c2nc(CCl)n(C)c2c1 ; COC(=O)c1cc(OC(F)F)c2nc(CN3CCC(n4nccc4OCc4ccc(Cl)cc4F)CC3)n(C)c2c1]

To a mixture of 5 (28 mg, 90.39 μmol, 1 eq) and 5A (27.54 mg, 90.39 μmol, 1 eq) in ACN (1 mL) was added K₂CO₃ (37.48 mg, 271.17 μmol, 3 eq). The mixture was stirred at 60° C. for 2 hours. LC-MS (ET83164-131-PIA, product: RT=0.459 min) showed 5 was consumed completely and one main peak with desired mass was detected. The mixture was concentrated in vacuum. The residue was purified by prep-TLC (SiO₂, PE/EtOAc=0/1). 6 (43 mg, 74.40 μmol, 82.31% yield) was obtained as a white solid.

LCMS: RT=0.459 min, MS cal.: 677.2, [M+H]⁺=678.4

¹H NMR (400 MHz, MeOH-d4) δ=8.13 (d, J=1.2 Hz, 1H) 7.67 (s, 1H) 7.51 (t, J=7.6 Hz, 1H) 7.24 (t, J=74 Hz, 1H) 7.25-7.32 (m, 3H) 5.76 (d, J=2.0 Hz, 1H) 5.19 (s, 2H) 4.09-4.21 (m, 1H) 4.00 (s, 3H) 3.96 (s, 3H) 3.90 (s, 2H) 2.92-3.02 (m, 2H) 2.24-2.39 (m, 2H) 2.11 (qd, J=12.4, 3.81 Hz, 2H) 1.72-1.86 (m, 2H)

### General Procedure for Preparation of Compound 200 (7):

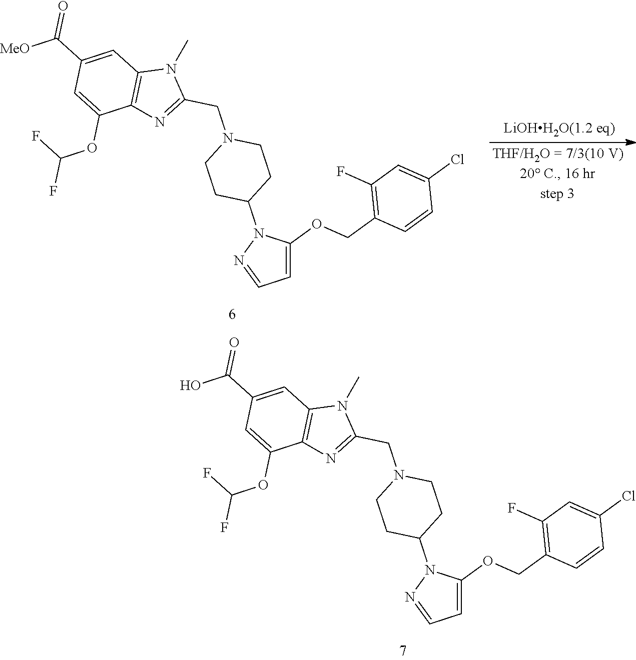 [SMILES: COC(=O)c1cc(OC(F)F)c2nc(CN3CCC(n4nccc4OCc4ccc(Cl)cc4F)CC3)n(C)c2c1 ; Cn1c(CN2CCC(n3nccc3OCc3ccc(Cl)cc3F)CC2)nc2c(OC(F)F)cc(C(=O)O)cc21]

To a mixture of 6 (43 mg, 74.40 μmol, 1 eq) in THF (2.1 mL) and H₂O (0.9 mL) was added LiOH·H₂O (3.75 mg, 89.28 μmol, 1.2 eq). The mixture was stirred at 20° C. for 16 hours. LC-MS (product: RT=1.095 min) showed 6 was consumed completely and one main peak with desired mass was detected. The mixture was concentrated in vacuum. The residue was purified by prep-HPLC (column: Waters Xbridge BEH C₁₈ 100*30 mm*10 um; mobile phase: [H₂O (10 mM NH₄HCO₃)-ACN]; gradient: 25%-55% B over 8.0 min). 2-((4-(5-((4-Chloro-2-fluorobenzyl)oxy)-1H-pyrazol-1-yl)piperidin-1-yl)methyl)-4-(difluoromethoxy)-1-methyl-1H-benzo[d]imidazole-6-carboxylic acid (26.21 mg, 46.31 μmol, 62.24% yield) was obtained as a white solid.

LCMS: RT=2.518 min, MS cal.: 563.2, [M+H]⁺=564.2

HPLC: RT=10.627 min, purity: 99.64%

¹H NMR (400 MHz, DMSO-d₆) δ=8.07 (d, J=0.8 Hz, 1H) 7.62 (t, J=8.0 Hz, 1H) 7.60 (t, J=74.4 Hz, 1H) 7.49-7.54 (m, 2H) 7.35 (dd, J=8.4, 1.6 Hz, 1H) 7.23 (d, J=1.6 Hz, 1H) 5.76 (d, J=1.6 Hz, 1H) 5.18 (s, 2H) 3.98-4.08 (m, 1H) 3.94 (s, 3H) 3.85 (s, 2H) 2.90 (br d, J=11.2 Hz, 2H) 2.23 (br t, J=11.2 Hz, 2H) 1.94 (qd, J=12.0, 3.6 Hz, 2H) 1.69 (br d, J=10.4 Hz, 2H)

### Example A127—Preparation of Compound 201. 4-(Difluoromethoxy)-2-((4-(6-((4-(difluoromethyl)-2-fluorobenzyl)oxy)pyridin-2-yl)piperidin-1-yl)methyl)-1-methyl-1H-benzo[d]imidazole-6-carboxylic acid (Compound 201) (3)

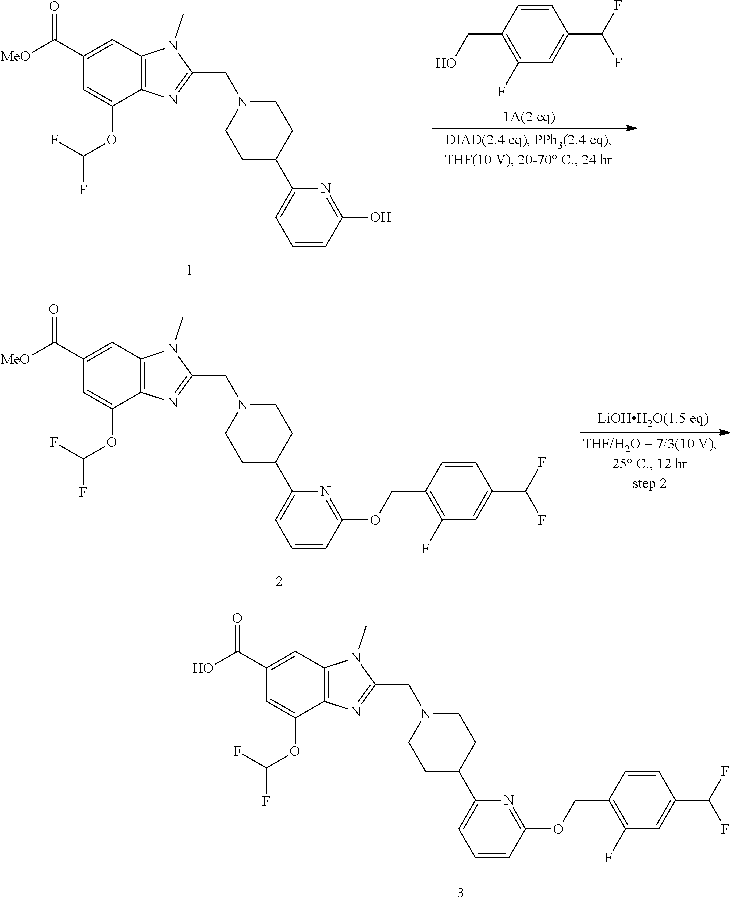 [SMILES: COC(=O)c1cc(OC(F)F)c2nc(CN3CCC(c4cccc(O)n4)CC3)n(C)c2c1 ; OCc1ccc(C(F)F)cc1F ; COC(=O)c1cc(OC(F)F)c2nc(CN3CCC(c4cccc(OCc5ccc(C(F)F)cc5F)n4)CC3)n(C)c2c1 ; Cn1c(CN2CCC(c3cccc(OCc4ccc(C(F)F)cc4F)n3)CC2)nc2c(OC(F)F)cc(C(=O)O)cc21]

### General Procedure for Preparation of Intermediate 2:

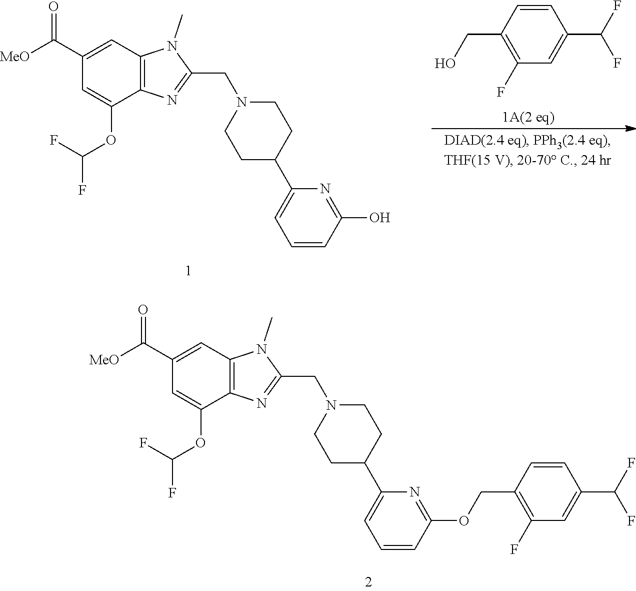 [SMILES: COC(=O)c1cc(OC(F)F)c2nc(CN3CCC(c4cccc(O)n4)CC3)n(C)c2c1 ; OCc1ccc(C(F)F)cc1F ; COC(=O)c1cc(OC(F)F)c2nc(CN3CCC(c4cccc(OCc5ccc(C(F)F)cc5F)n4)CC3)n(C)c2c1]

To a solution of 1 (150 mg, 335.99 μmol, 1 eq) and 1A (118.36 mg, 671.97 μmol, 2 eq) in THF (0.75 mL) was added DIAD (81.53 mg, 403.18 μmol, 78.17 μL, 1.2 eq) and PPh₃ (105.75 mg, 403.18 μmol, 1.2 eq) in THF (0.75 mL) at 20° C. The mixture was stirred at 70° C. for 12 hr. At 20° C., the mixture was added DIAD (81.53 mg, 403.18 μmol, 78.17 μL, 1.2 eq) and PPh₃ (105.75 mg, 403.18 μmol, 1.2 eq) in THF (0.5 mL). The mixture was stirred at 70° C. for 12 hr. LCMS (product: RT=1.656 min) showed the 1 was consumed completely. The reaction mixture was cooled down to 20° C. and quenched by addition H₂O 2 mL at 20° C. and extracted with ethyl acetate 3 mL*3. The combined organic layers were washed with brine 3 mL*2, dried over Na₂SO₄, filtered and concentrated under reduced pressure to give a residue. The residue was purified by column chromatography (SiO₂, Petroleum ether/Ethyl acetate=10/1 to 0/1). 2 (80 mg, 132.33 μmol, 39.38% yield) was obtained as a yellow solid.

LCMS: RT=1.673 min, MS cal.: 604.2, [M+H]⁺=605.2

### General Procedure for Preparation of Compound 20 (3):

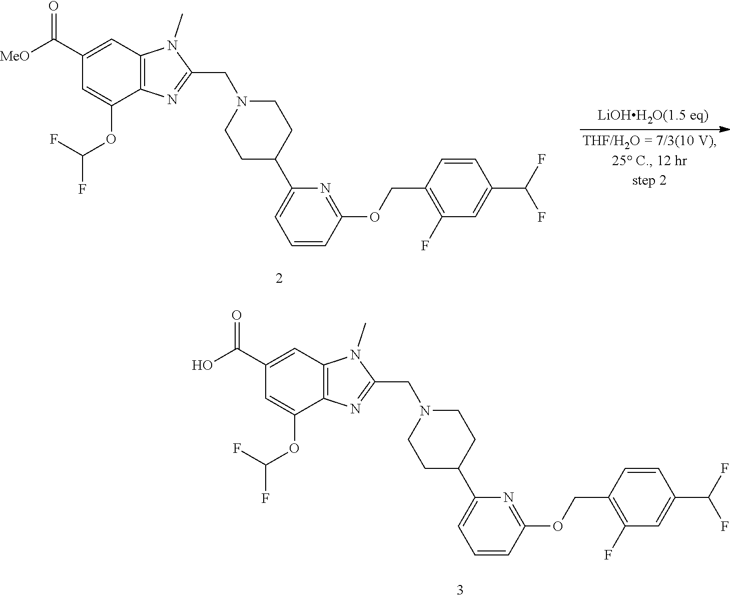 [SMILES: COC(=O)c1cc(OC(F)F)c2nc(CN3CCC(c4cccc(OCc5ccc(C(F)F)cc5F)n4)CC3)n(C)c2c1 ; Cn1c(CN2CCC(c3cccc(OCc4ccc(C(F)F)cc4F)n3)CC2)nc2c(OC(F)F)cc(C(=O)O)cc21]

A mixture of 2 (80 mg, 132.33 μmol, 1 eq), LiOH·H₂O (8.33 mg, 198.49 μmol, 1.5 eq) in THF (0.56 mL) and H₂O (0.24 mL) was degassed and purged with N₂ for 3 times, and then the mixture was stirred at 25° C. for 12 hr under N₂ atmosphere. LCMS (product: RT=1.182 min) showed the 2 was consumed completely. The reaction mixture was filtered, and the filtrate was purified by prep-HPLC (column: Waters Xbridge Prep OBD C18 150*40 mm*10 um; mobile phase: [H₂O (10 mM NH₄HCO₃)-ACN]; gradient: 30%-80% B over 8.0 min). 4-(Difluoromethoxy)-2-((4-(6-((4-(difluoromethyl)-2-fluorobenzyl)oxy)pyridin-2-yl)piperidin-1-yl)methyl)-1-methyl-1H-benzo[d]imidazole-6-carboxylic acid (28.40 mg, 47.41 μmol, 35.83% yield, 98.59% purity) was obtained as a white solid.

LCMS: RT=2.733 min, MS cal.: 590.2, [M+H]⁺=591.2

HPLC: RT=11.746 min

¹H NMR (400 MHz, MeOH-d4) δ=8.10 (d, J=1.6 Hz, 1H), 7.68 (s, 1H), 7.64-7.55 (m, 2H), 7.31 (t, J=10.4 Hz, 2H), 7.25 (t, J=74.0 Hz, 1H), 6.83 (d, J=7.6 Hz, 1H), 6.75 (t, J=56.0 Hz, 1H), 6.66 (d, J=8.0 Hz, 1H), 5.47 (s, 2H), 4.02 (s, 3H), 3.99 (s, 2H), 3.10 (d, J=11.6 Hz, 2H), 2.73-2.61 (m, 1H), 2.49-2.36 (m, 2H), 1.95-1.84 (m, 4H)

### Example A128—Preparation of Compound 202. 2-((4-(2-((4-Cyano-2-fluorobenzyl)oxy)-3-fluorophenyl)piperidin-1-yl)methyl)-4-(difluoromethoxy)-1-methyl-1H-benzo[d]imidazole-6-carboxylic acid (Compound 202) (5)

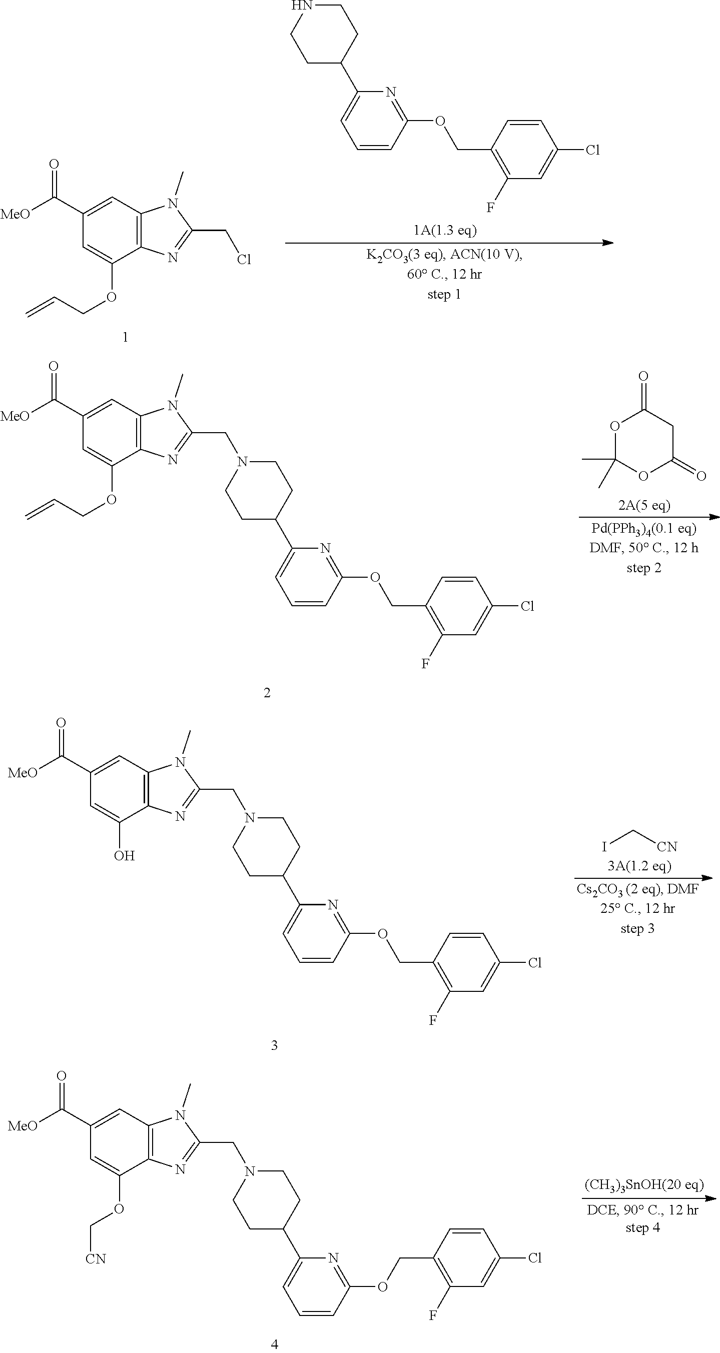

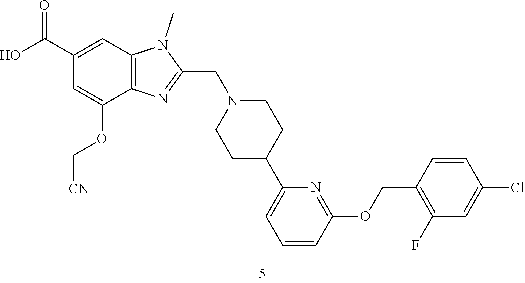 [SMILES: Cn1c(CN2CCC(c3cccc(OCc4ccc(Cl)cc4F)n3)CC2)nc2c(OCC#N)cc(C(=O)O)cc21]

### General Procedure for Preparation of Intermediate 2:

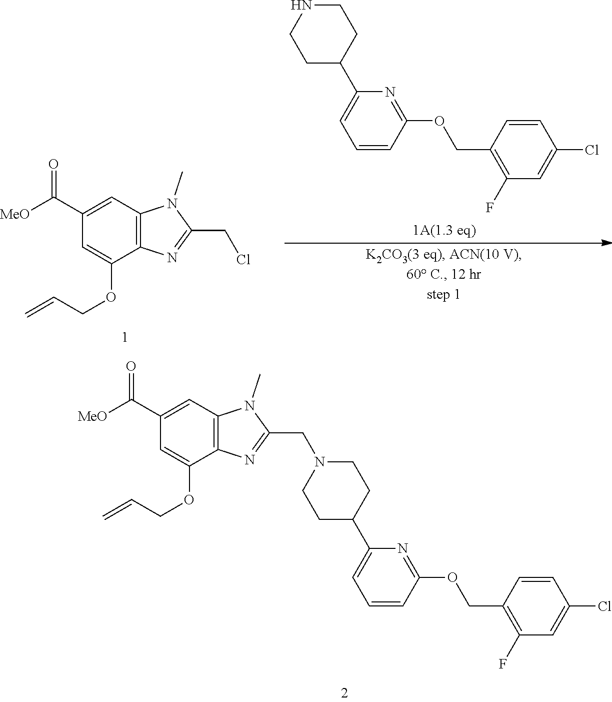 [SMILES: C=CCOc1cc(C(=O)OC)cc2c1nc(CCl)n2C ; Fc1cc(Cl)ccc1COc1cccc(C2CCNCC2)n1 ; C=CCOc1cc(C(=O)OC)cc2c1nc(CN1CCC(c3cccc(OCc4ccc(Cl)cc4F)n3)CC1)n2C]

To a solution of 1 (1 g+0.33 g, 3.39 mmol, 1 eq, from INT 5) in DMF (10 mL) was added K₂CO₃ (1.41 g, 10.18 mmol, 3 eq) and 1A (1.20 g, 3.73 mmol, 1.1 eq) at 25° C. The mixture was stirred at 60° C. for 2 hr. LC-MS showed 1 was consumed completely and one main peak with desired mass was detected. The mixture was diluted with H₂O (50 mL), extracted with EtOAc (50 mL*3). The combined organic layer was washed with 50 mL H₂O, 50 mL brine, dried over Na₂SO₄, filtered and the filtrate was concentrated to give a crude product. The residue was purified by column chromatography (SiO₂, PE/EtOAc=10/1 to 0/1). 2 (1.8 g, 3.11 mmol, 91.62% yield) was obtained as a yellow solid.

### General Procedure for Preparation of Intermediate 3:

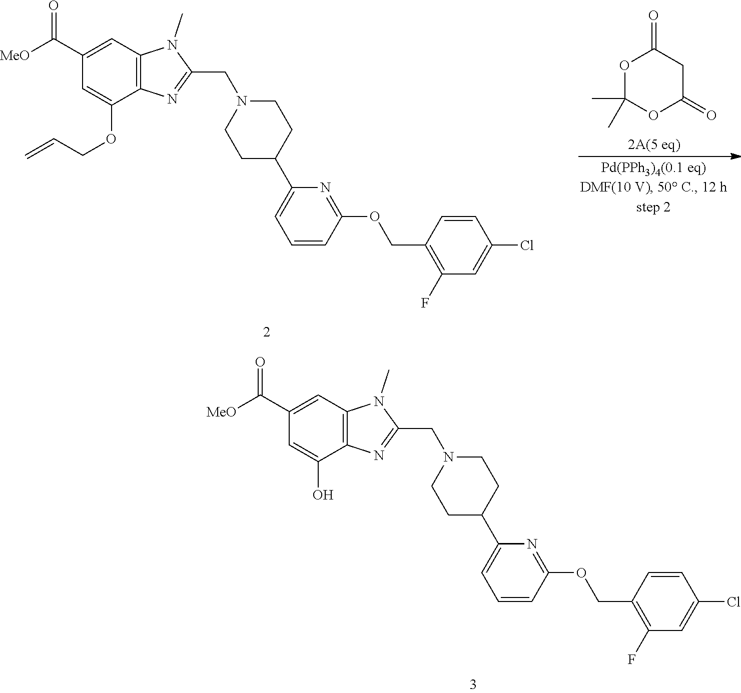

To a solution of 2 (1.1 g, 1.90 mmol, 1 eq) and 2A (1.37 g, 9.50 mmol, 5 eq) in DMF (15 mL) was added Pd(PPh₃)₄ (219.51 mg, 189.96 μmol, 0.1 eq). The mixture was stirred at 50° C. for 12 h under N₂ atmosphere. LCMS showed 2 was consumed completely and one main peak with desired mass was detected. The reaction mixture was quenched by addition water (30 mL), and then extracted with EA (20 mL*2). The combined organic layers were washed with NaCl (30 mL*2), dried over Na₂SO₄, filtered and concentrated under reduced pressure to give a residue. The residue was purified by prep-HPLC (column: Agela DuraShell C18 250*70 mm*10 um; mobile phase: [H₂O (10 mM NH₄HCO₃)-ACN]; gradient: 55%-85% B over 17.0 min). 3 (600 mg, 1.11 mmol, 53.7% yield) was obtained as a light-yellow solid.

LCMS: RT=0.456 min, MS cal.: 538.2, [M+1]⁺=539.2

¹H NMR (400 MHz, DMSO-d₆) δ=10.12 (s, 1H), 7.65-7.60 (m, 2H), 7.55 (t, J=8.0 Hz, 1H), 7.45 (dd, J=10.0, 2.0 Hz, 1H), 7.28 (dd, J=8.4, 1.6 Hz, 1H), 7.19 (d, J=1.2 Hz, 1H), 6.86 (d, J=7.2 Hz, 1H), 6.66 (d, J=8.0 Hz, 1H), 5.36 (s, 2H), 3.89 (s, 3H), 3.85 (s, 3H), 3.81 (s, 2H), 2.94 (d, J=11.2 Hz, 2H), 2.62-2.55 (m, 1H), 2.19 (t, J=11.2 Hz, 2H), 1.84-1.63 (m, 4H)

### General Procedure for Preparation of Intermediate 4:

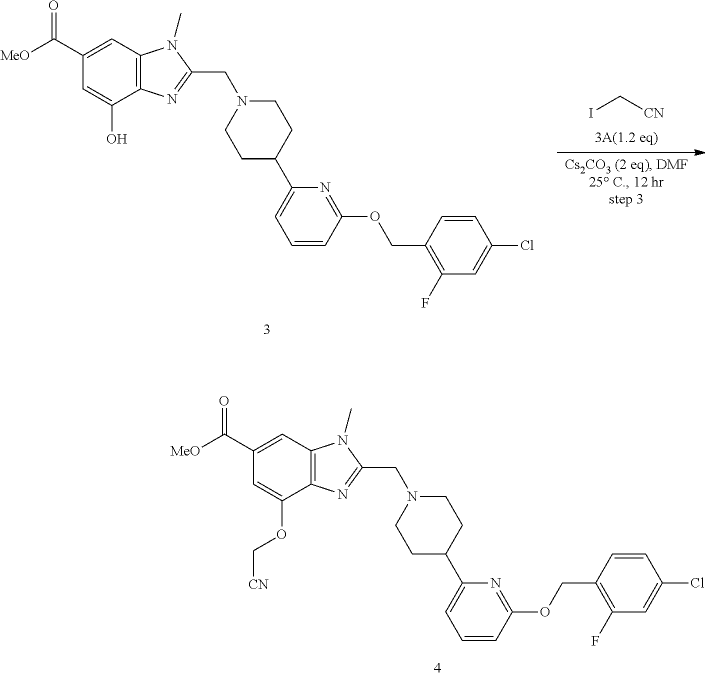

To a solution of 3 (60 mg, 111.32 μmol, 1 eq) and 3A (22.30 mg, 133.58 μmol, 1.2 eq) in DMF (2 mL) was added Cs₂CO₃ (72.54 mg, 222.64 μmol, 2 eq). The mixture was stirred at 25° C. for 12 hr under N₂ atmosphere. LC-MS showed 3 was consumed completely and one main peak with desired mass was detected. The reaction mixture was quenched by addition water 5 mL, and then extracted with EtOAc (5 mL*3). The combined organic layers were washed with brine (10 mL), dried over Na₂SO₄, filtered and concentrated under reduced pressure to give a residue. The crude product was not purified and used to next step directly. 4 (60 mg, 103.80 μmol, 80% yield) as a yellow solid.

LCMS: RT=0.476 min, MS cal.: 577.2, [M+H]=578.2

¹H NMR (400 MHz, DMSO-d₆) δ=7.97 (d, J=1.2 Hz, 1H), 7.65-7.59 (m, 1H), 7.55 (t, J=8.0 Hz, 1H), 7.45 (dd, J=10.0, 2.0 Hz, 1H), 7.41 (d, J=1.2 Hz, 1H), 7.28 (dd, J=8.4, 1.6 Hz, 1H), 6.86 (d, J=7.6 Hz, 1H), 6.66 (d, J=8.4 Hz, 1H), 5.49 (s, 2H), 5.35 (s, 2H), 3.95 (s, 3H), 3.90 (s, 3H), 3.84 (s, 2H), 2.94 (d, J=11.2 Hz, 2H), 2.63-2.55 (m, 1H), 2.21 (t, J=9.6 Hz, 2H), 1.82-1.65 (m, 4H)

### General Procedure for Preparation of Compound 202 (5):

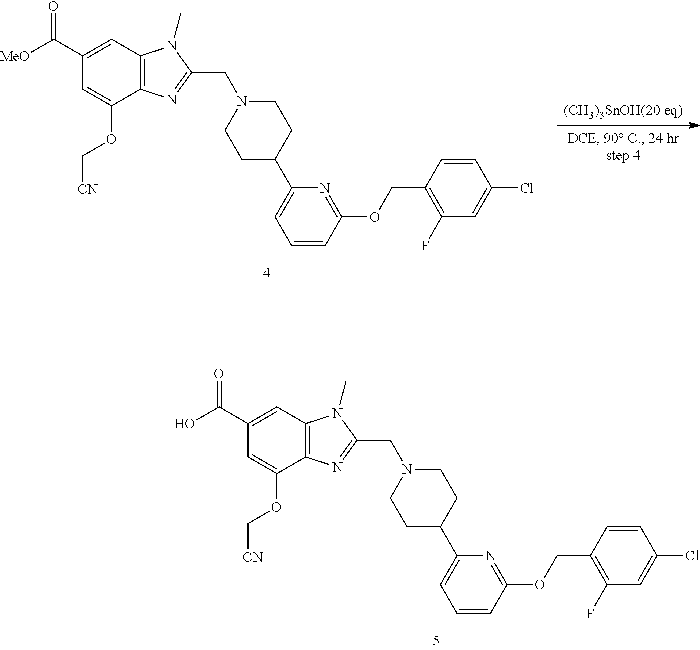 [SMILES: COC(=O)c1cc(OCC#N)c2nc(CN3CCC(c4cccc(OCc5ccc(Cl)cc5F)n4)CC3)n(C)c2c1 ; "(CH3)3SnOH(20 eq)" ; "DCE, 90° C., 24 hrstep 4" ; Cn1c(CN2CCC(c3cccc(OCc4ccc(Cl)cc4F)n3)CC2)nc2c(OCC#N)cc(C(=O)O)cc21]

To a solution of 4 (35 mg, 60.55 μmol, 1 eq) in DCE (1 mL) was added (CH₃)₃SnOH (109.49 mg, 605.50 μmol, 10 eq) at 20° C. The mixture was stirred at 90° C. for 12 hr. (CH₃)₃SnOH (109.49 mg, 605.50 μmol, 10 eq) was added to the mixture. The mixture was stirred at 90° C. for 12 hr. LC-MS showed 4 was consumed completely and one main peak with desired mass was detected. The reaction mixture was added saturated KF solution (2 ml) at 20° C., the mixture was diluted with H₂O 2 mL and extracted with DCM (2 mL*3). The combined organic layers were dried over Na₂SO₄, filtered, and concentrated under reduced pressure to give a residue. The residue was purified by prep-HPLC (column: 3_Phenomenex Luna C18 75*30 mm*3 um; mobile phase: [H₂O (0.2% FA)-ACN]; gradient: 30%-60% B over 8.0 min). 2-((4-(2-((4-Cyano-2-fluorobenzyl)oxy)-3-fluorophenyl)piperidin-1-yl)methyl)-4-(difluoromethoxy)-1-methyl-1H-benzo[d]imidazole-6-carboxylic acid (3.5 mg, 6.21 μmol, 10.25% yield) was obtained as a white solid.

LCMS: RT=2.344 min, MS cal.: 563.1, [M+H]⁺=564.2

HPLC: RT=0.2.687 min, purity: 99.83%

¹H NMR (400 MHz, DMSO-d₆) δ=13.15 (s, 1H), 7.92 (s, 1H), 7.62 (t, J=7.6 Hz, 1H), 7.55 (t, J=8.0 Hz, 1H), 7.45 (dd, J=10.0, 2.0 Hz, 1H), 7.41 (s, 1H), 7.28 (dd, J=8.4, 2.0 Hz, 1H), 6.86 (d, J=7.2 Hz, 1H), 6.66 (d, J=8.0 Hz, 1H), 5.47 (s, 2H), 5.36 (s, 2H), 3.94 (s, 3H), 3.83 (s, 2H), 2.94 (d, J=11.2 Hz, 2H), 2.59 (s, 1H), 2.27-2.16 (m, 2H), 1.82-1.65 (m, 4H)

### Example A129—Preparation of Compound 205. 2-((4-(1-(4-Chloro-2-fluorobenzyl)-1H-pyrrolo[2,3-b]pyridin-6-yl)piperidin-1-yl)methyl)-4-(difluoromethoxy)-1-methyl-1H-benzo[d]imidazole-6-carboxylic acid (Compound 205) (7)

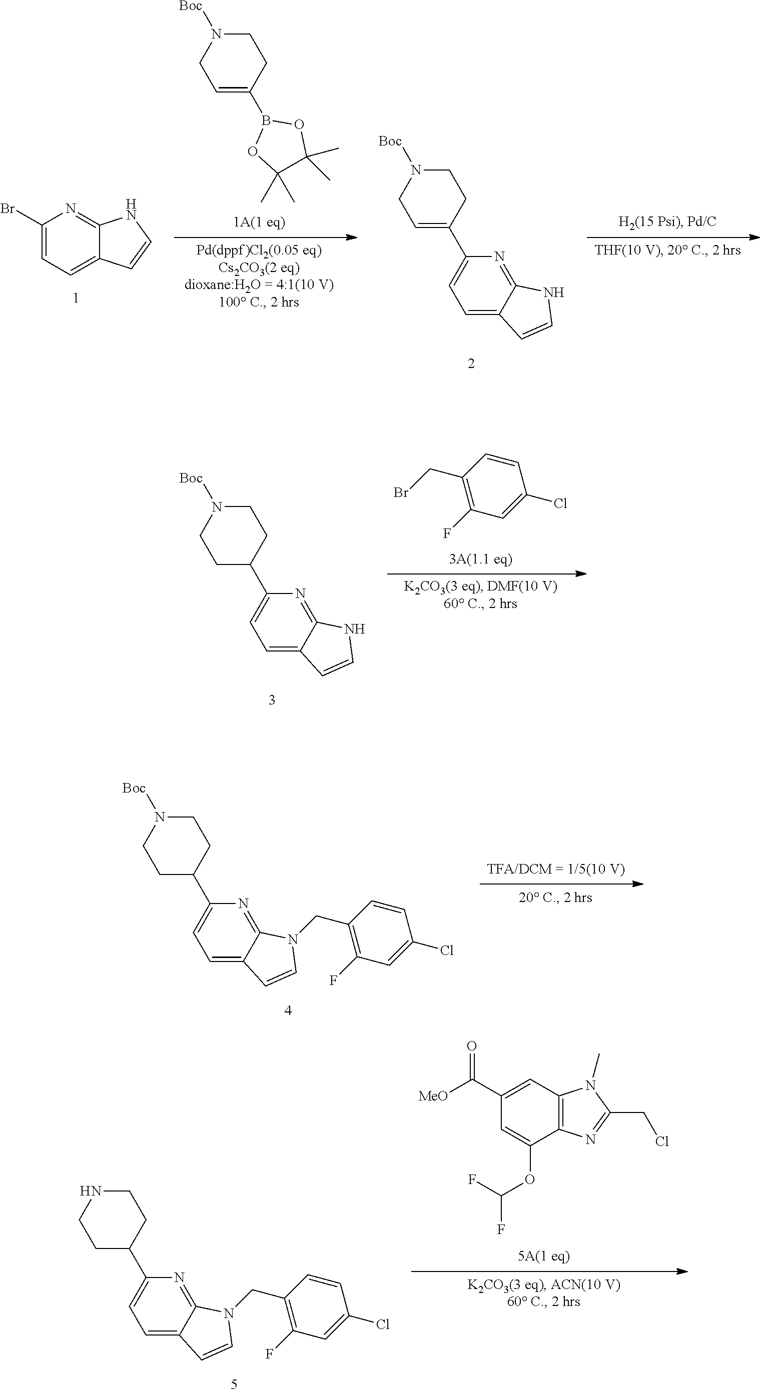

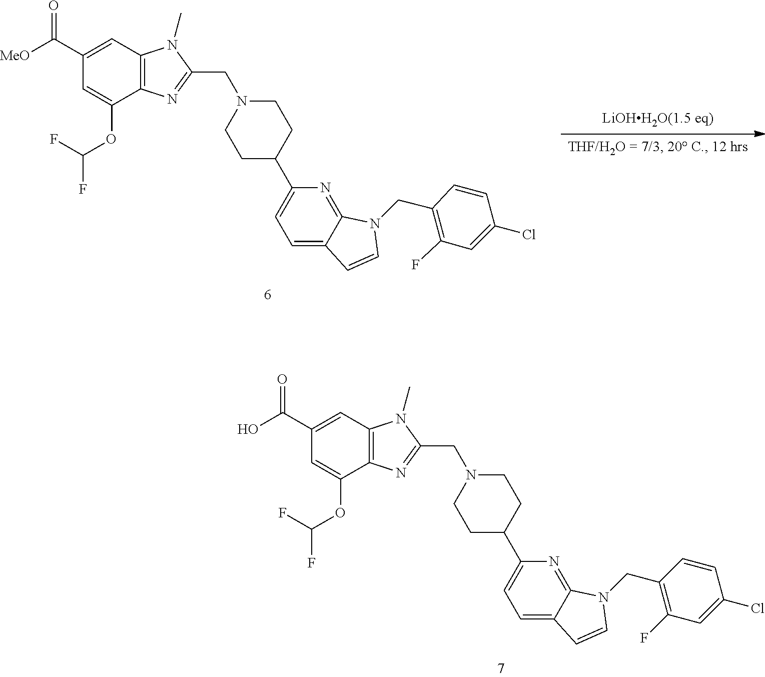

### General Procedure for Preparation of Intermediate 2:

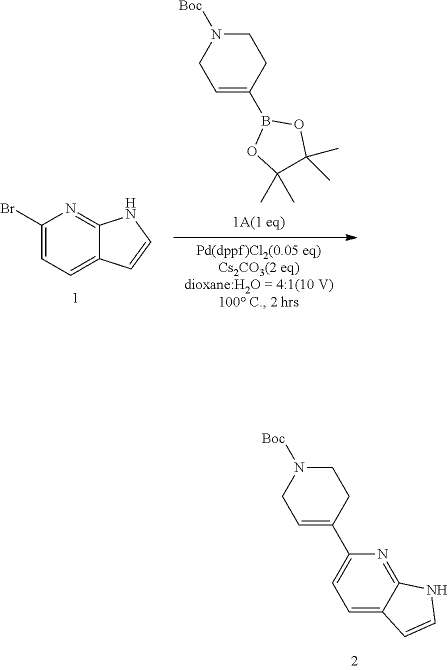

A mixture of 1A (3.14 g, 10.15 mmol, 1 eq), 1 (2 g, 10.15 mmol, 1 eq), Pd(dppf)Cl₂ (371.36 mg, 507.53 μmol, 0.05 eq), Cs₂CO₃ (6.61 g, 20.30 mmol, 2 eq) in dioxane (20 mL) and H₂O (5 mL) was degassed and purged with N₂ for 3 times, and then the mixture was stirred at 100° C. for 2 hrs under N₂ atmosphere. LC-MS showed 1 was consumed completely and desired mass was detected. The suspension was filtered through a pad of Celite and filter cake was washed with EtOAc (5 mL*3). The combined filtrates were diluted with H₂O 30 mL and extracted with EtOAc 60 mL (20 mL*3). The combined organic layers were washed with brine 15 mL, dried over Na₂SO₄, filtered and concentrated under reduced pressure to give a residue. The residue was purified by column chromatography (SiO₂, Petroleum ether/Ethyl acetate=20/1 to 1/1). 2 (2 g, 6.68 mmol, 65.82% yield) was obtained as a white solid.

LCMS: RT=1.430 min, MS cal.: 299.16, [M+H]⁺=300.2

¹H NMR (400 MHz, DMSO-d6) δ=11.59 (br s, 1H), 7.90 (d, J=8.4 Hz, 1H), 7.45-7.40 (m, 1H), 7.31 (d, J=8.4 Hz, 1H), 6.58 (br s, 1H), 6.41 (dd, J=1.2, 3.2 Hz, 1H), 4.05 (br s, 2H), 3.55 (br t, J=5.6 Hz, 2H), 2.64 (br s, 2H), 1.43 (s, 9H)

### General Procedure for Preparation of Intermediate 3:

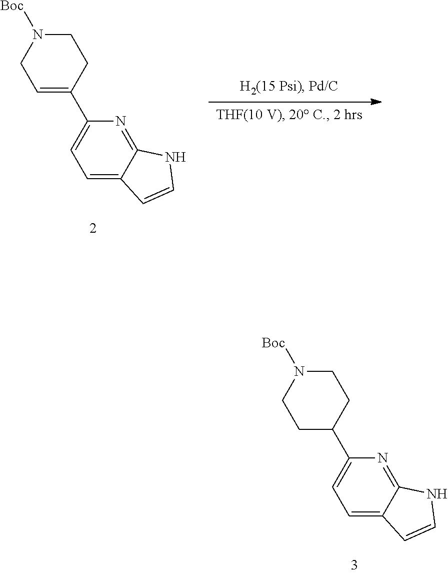 [SMILES: CC(C)(C)OC(=O)N1CC=C(c2ccc3cc[nH]c3n2)CC1 ; "H2(15 Psi), Pd/C" ; "THF(10 V), 20° C., 2 hrs" ; CC(C)(C)OC(=O)N1CCC(c2ccc3cc[nH]c3n2)CC1]

THF (5 mL) was charged to the round bottom flask, then 2 (500 mg, 1.67 mmol, 1 eq) was added to the mixture at 20° C. At 20° C., Pd/C (200 mg, 10% purity) under N₂ atmosphere. The suspension was degassed and purged with H₂ for 3 times. The mixture was stirred under H₂ (15 Psi) at 20° C. for 2 hrs. LC-MS showed 2 was consumed completely and desired mass was detected. The mixture was filtered through a Celite pad and washed by THF (5 mL*3), and the filtrate was concentrated to give the crude product. 3 (480 mg, 1.59 mmol, 95.36% yield) was obtained as a white solid.

LCMS: RT=1.899 min, MS cal.: 301.1, [M+H]⁺=302.2

¹H NMR (400 MHz, DMSO-d6) δ=11.48 (br s, 1H), 7.85 (d, J=8.0 Hz, 1H), 7.43-7.30 (m, 1H), 6.96 (d, J=8.0 Hz, 1H), 6.37 (dd, J=1.6, 3.2 Hz, 1H), 4.12-4.02 (m, 2H), 2.94-2.78 (m, 3H), 1.84 (br d, J=11.6 Hz, 2H), 1.71-1.56 (m, 2H), 1.42 (s, 9H)

### General Procedure for Preparation of Intermediate 4:

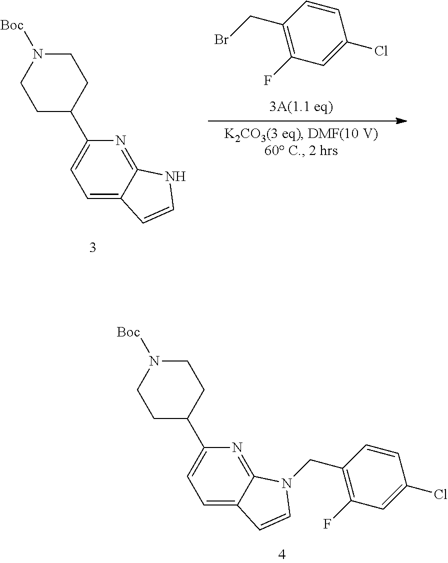

To a solution of 3 (200 mg, 663.61 μmol, 1 eq) in DMF (2 mL) was added K₂CO₃ (275.14 mg, 1.99 mmol, 3 eq) and 3A (163.13 mg, 729.97 μmol, 1.1 eq). The mixture was stirred at 60° C. for 2 hrs. LC-MS showed 3 was consumed completely and desired mass was detected. The residue was diluted with H₂O 15 mL and extracted with DCM 60 mL (20 mL*3). The combined organic layers were washed with brine 10 mL, dried over Na₂SO₄, filtered, and concentrated under reduced pressure to give a residue. The residue was purified by column chromatography (SiO₂, Petroleum ether/Ethyl acetate=10/1 to 0/1). 4 (170 mg, 382.93 μmol, 57.70% yield) was obtained as colorless oil

¹H NMR (400 MHz, DMSO-d6) δ=7.89 (d, J=8.0 Hz, 1H), 7.49 (d, J=3.4 Hz, 1H), 7.43 (dd, J=1.8, 9.9 Hz, 1H), 7.24-7.14 (m, 2H), 7.02 (d, J=8.0 Hz, 1H), 6.46 (d, J=3.5 Hz, 1H), 5.47 (s, 2H), 4.05 (br d, J=11.3 Hz, 2H), 2.97-2.78 (m, 3H), 1.82 (br d, J=10.8 Hz, 2H), 1.71-1.53 (m, 2H), 1.42 (s, 9H)

### General Procedure for Preparation of Intermediate 5:

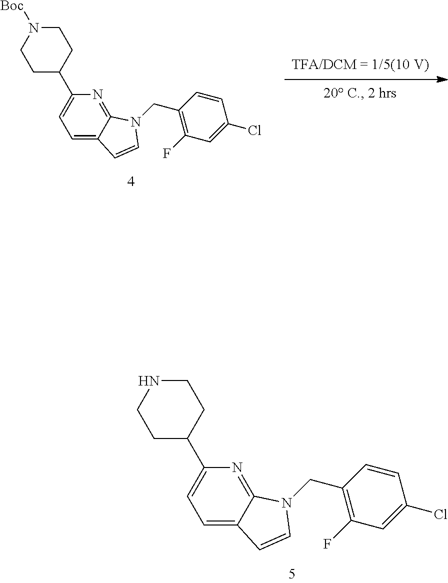

To a solution of 4 (150 mg, 337.88 μmol, 1 eq) in DCM (1.5 mL) was added TFA (460.50 mg, 4.04 mmol, 0.3 mL, 11.95 eq). The mixture was stirred at 20° C. for 2 hrs. LC-MS showed 4 was consumed completely and desired mass was detected. The reaction mixture was concentrated under reduced pressure to remove solvent. 5 (150 mg, 327.62 μmol, 96.96% yield, TFA) was obtained as colorless oil.

LCMS: RT=1.396 min, MS cal.: 343.1, [M+H]⁺=344.1

### General Procedure for Preparation of Intermediate 6:

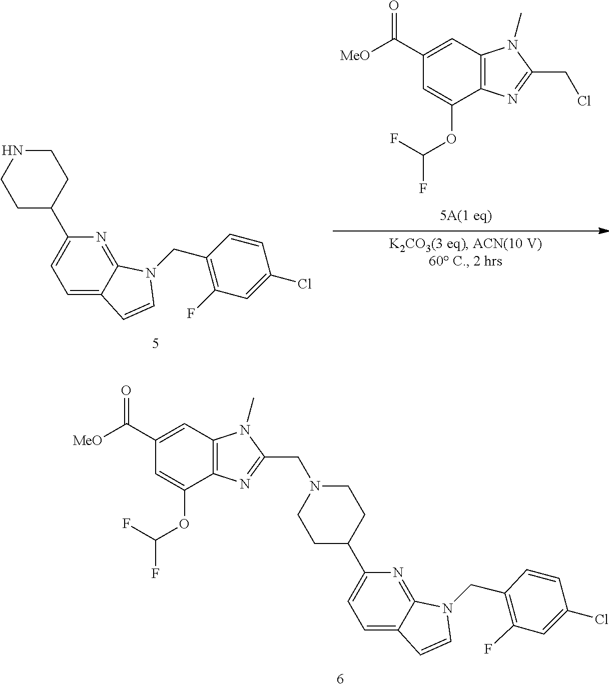 [SMILES: Fc1cc(Cl)ccc1Cn1ccc2ccc(C3CCNCC3)nc21 ; COC(=O)c1cc(OC(F)F)c2nc(CCl)n(C)c2c1 ; "5A(1 eq)" ; "K2CO3(3 eq), ACN(10 V)60° C., 2 hrs" ; COC(=O)c1cc(OC(F)F)c2nc(CN3CCC(c4ccc5ccn(Cc6ccc(Cl)cc6F)c5n4)CC3)n(C)c2c1]

To a solution of 5 (150 mg, 327.62 μmol, 1 eq, TFA) in ACN (1.5 mL) was added K₂CO₃ (135.84 mg, 982.86 μmol, 3 eq) and 5A (99.82 mg, 327.62 μmol, 1 eq). The mixture was stirred at 60° C. for 2 hrs. LC-MS showed 5 was consumed completely and desired mass was detected. The residue was diluted with H₂O 15 mL and extracted with EtOAc 45 mL (15 mL*3). The combined organic layers were washed with brine 15 mL, dried over Na₂SO₄, filtered, and concentrated under reduced pressure to give a residue. The residue was purified by column chromatography (SiO₂, Petroleum ether/Ethyl acetate=10/1 to 0/1). 6 (120 mg, 196.07 μmol, 59.85% yield) was obtained as a white solid.

LCMS: RT=1.789 min, MS cal.: 611.1, [M+H]⁺=612.3

¹H NMR (400 MHz, DMSO-d6) δ=8.11 (d, J=1.2 Hz, 1H), 7.90-7.82 (m, 1H), 7.65 (t, J=74.4 Hz, 1H), 7.53 (s, 1H), 7.47 (d, J=4.0 Hz, 1H), 7.42 (dd, J=1.6, 10.0 Hz, 1H), 7.25-7.12 (m, 2H), 7.00 (d, J=8.0 Hz, 1H), 6.44 (d, J=3.2 Hz, 1H), 5.46 (s, 2H), 3.99 (s, 3H), 3.90 (s, 3H), 3.86 (s, 2H), 2.96 (br d, J=11.2 Hz, 2H), 2.72 (br dd, J=4.8, 10.2 Hz, 1H), 2.28-2.20 (m, 2H), 1.89-1.73 (m, 4H)

### General Procedure for Preparation of Compound 205 (7):

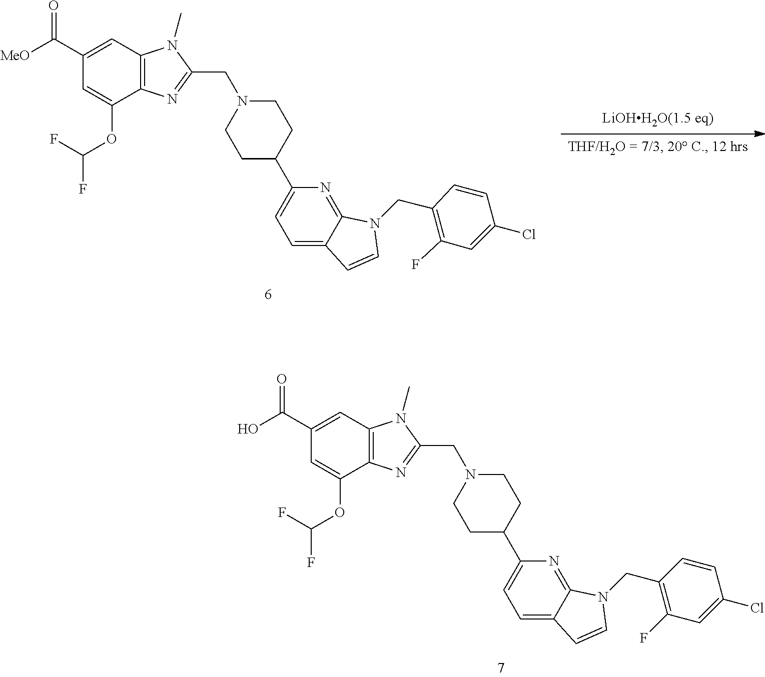

To a solution of 6 (110 mg, 179.73 μmol, 1 eq) in THF (0.7 mL) was added LiOH·H₂O (11.31 mg, 269.59 μmol, 1.5 eq) in H₂O (0.3 mL). The mixture was stirred at 20° C. for 12 hrs. LC-MS showed ˜29% of 6 remained. Several new peaks were shown on LC-MS and ˜67% of desired Compound was detected. The reaction was added citric acid (0.5 M) to pH=6-7, then the mixture was purified directly. The residue was purified by prep-HPLC (neutral condition; column: Waters Xbridge Prep OBD C18 150*40 mm*10 um; mobile phase: [H₂O (10 mM NH₄HCO₃)-ACN]; gradient: 15%-65% B over 8.0 min). 2-((4-(1-(4-Chloro-2-fluorobenzyl)-1H-pyrrolo[2,3-b]pyridin-6-yl)piperidin-1-yl)methyl)-4-(difluoromethoxy)-1-methyl-1H-benzo[d]imidazole-6-carboxylic acid (32.62 mg, 53.40 μmol, 29.71% yield, 97.9% purity) was obtained as a white solid.

LCMS: RT=2.511 min, MS cal.: 597.1, [M+H]⁺=598.2

HPLC: RT=12.471 min, purity: 97.90%

¹H NMR (400 MHz, MeOH-d4) δ=8.13 (s, 1H), 7.87 (d, J=8.0 Hz, 1H), 7.71 (s, 1H), 7.32 (d, J=3.6 Hz, 1H), 7.29 (t, J=74.4 Hz, 1H), 7.22 (d, J=10.4 Hz, 1H), 7.12-7.07 (m, 2H), 7.04 (d, J=8.0 Hz, 1H), 6.46 (d, J=3.6 Hz, 1H), 5.53 (s, 2H), 4.07 (s, 2H), 4.04 (s, 3H), 3.21 (br d, J=11.2 Hz, 2H), 2.94-2.82 (m, 1H), 2.54 (dt, J=3.2, 11.2 Hz, 2H), 2.10-1.95 (m, 4H)

### Example A130—Preparation of Compound 207. 2-((4-(6-((4-chloro-2-fluorobenzyl)oxy)pyridin-2-yl)piperidin-1-yl)methyl)-4-(methoxy-d3)-1-methyl-1H-benzo[d]imidazole-6-carboxylic acid (Compound 207) (3)

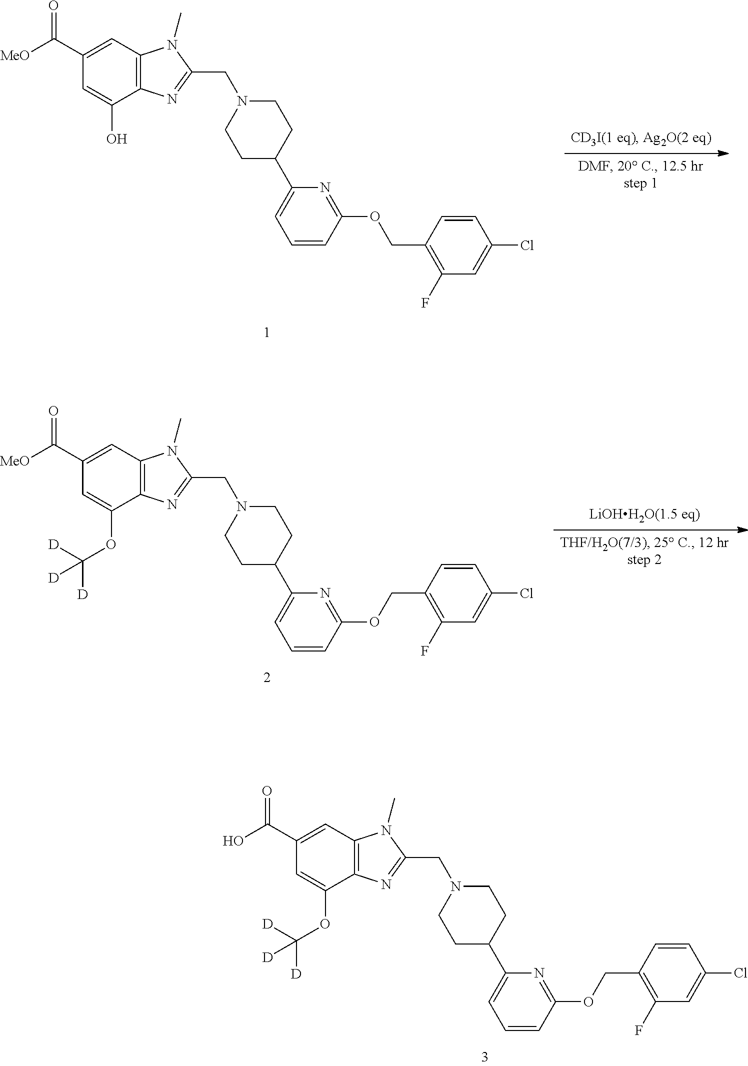

### General Procedure for Preparation of Intermediate 2:

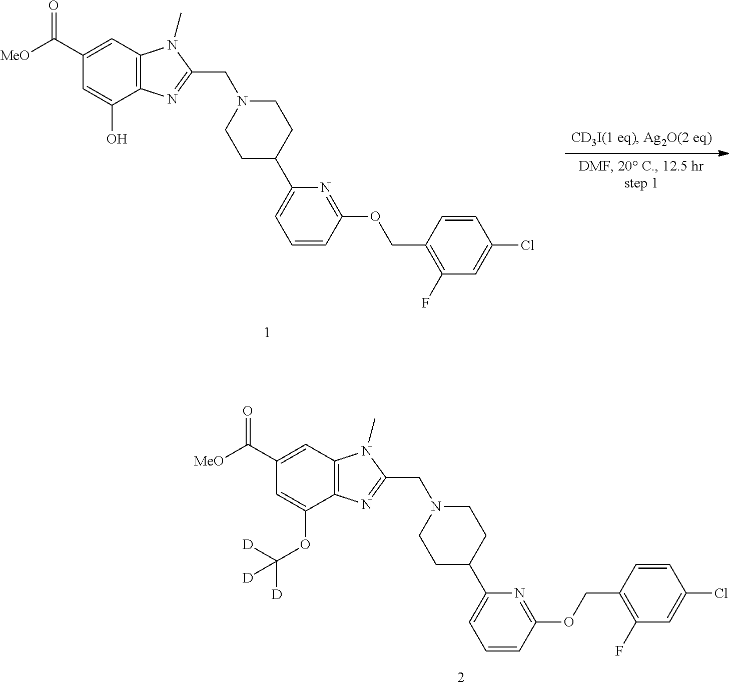 [SMILES: COC(=O)c1cc(O)c2nc(CN3CCC(c4cccc(OCc5ccc(Cl)cc5F)n4)CC3)n(C)c2c1 ; "CD3I(1 eq), Ag2O(2 eq)" ; "DMF, 20° C., 12.5 hrstep 1" ; [2H]C([2H])([2H])Oc1cc(C(=O)OC)cc2c1nc(CN1CCC(c3cccc(OCc4ccc(Cl)cc4F)n3)CC1)n2C]

To a solution of 1 (60 mg, 111.32 μmol, 1 eq) in DMF (1.5 mL) was added Ag₂O (51.59 mg, 222.64 μmol, 2 eq). The mixture was stirred at 20° C. for 0.5 hr, then CD₃I (15.80 mg, 111.32 μmol, 6.78 μL, 1 eq) was added to the mixture at 20° C. The mixture was stirred at 20° C. for 12 hr. LC-MS showed 1 was consumed completely and one main peak with desired mass was detected. The reaction mixture was quenched by addition water 5 mL, and then extracted with EtOAc 10 mL (5 mL*2). The combined organic layers were washed with brine 20 mL (10 mL*2), dried over Na₂SO₄, filtered, and concentrated under reduced pressure to give a residue. 2 (51 mg, 91.72 μmol, 61.8% yield) was obtained as a brown solid.

LCMS: RT=0.486 min, MS cal.: 556.05, [M+H]⁺=556.3

¹H NMR (400 MHz, DMSO-d₆) δ=7.83 (d, J=1.2 Hz, 1H), 7.65-7.59 (m, 1H), 7.56 (t, J=8.0 Hz, 1H), 7.45 (dd, J=1.6, 2.0 Hz, 1H), 7.29-7.25 (m, 1H), 6.87 (d, J=2.8 Hz, 1H), 6.66 (d, J=8.4 Hz, 1H), 5.36 (s, 2H), 3.92 (s, 3H), 3.88 (s, 3H), 3.82 (s, 2H), 2.93 (br d, J=11.2 Hz, 2H), 2.61-2.56 (m, 1H), 2.24-2.17 (m, 2H), 1.81-1.70 (m, 4H)

### General Procedure for Preparation of Compound 207 (3):

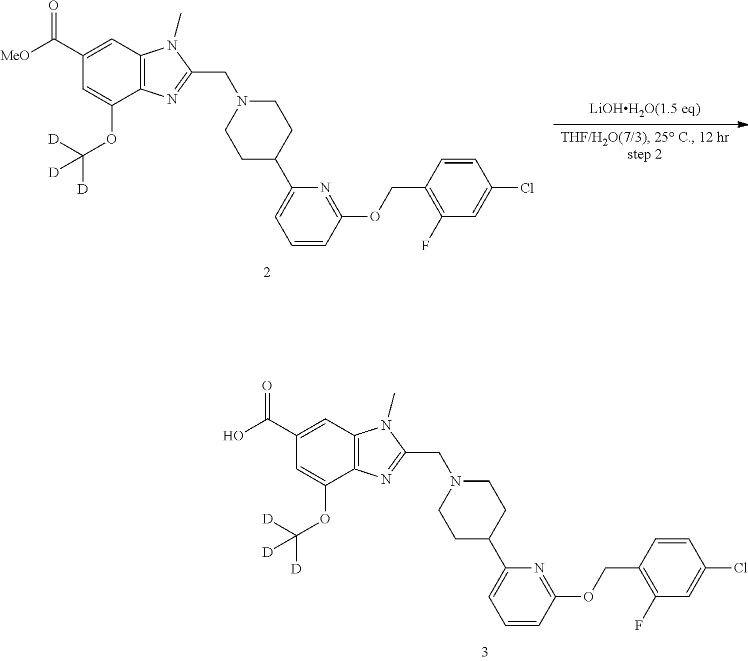 [SMILES: [2H]C([2H])([2H])Oc1cc(C(=O)OC)cc2c1nc(CN1CCC(c3cccc(OCc4ccc(Cl)cc4F)n3)CC1)n2C ; "LiOH·H2O(1.5 eq)" ; "THF/H2O(7/3), 25° C., 12 hrstep 2" ; [2H]C([2H])([2H])Oc1cc(C(=O)O)cc2c1nc(CN1CCC(c3cccc(OCc4ccc(Cl)cc4F)n3)CC1)n2C]

A mixture of 2 (40 mg, 71.94 μmol, 1 eq), LiOH·H₂O (4.53 mg, 107.91 μmol, 1.5 eq) in THF (0.7 mL) and H₂O (0.3 mL) was degassed and purged with N₂ for 3 times, and then the mixture was stirred at 25° C. for 12 hr under N₂ atmosphere. LC-MS (RT=1.171 min) showed 2 was consumed completely and desired mass was detected. The reaction mixture was filtered and concentrated under reduced pressure to give a residue. The crude product was purified by prep-HPLC (column: Waters Xbridge Prep OBD C18 150*40 mm*10 um; mobile phase: [H₂O (10 mM NH₄HCO₃)-ACN]; gradient: 25%-60% B over 8.0 min). 2-((4-(6-((4-Chloro-2-fluorobenzyl)oxy)pyridin-2-yl)piperidin-1-yl)methyl)-4-(methoxy-d3)-1-methyl-1H-benzo[d]imidazole-6-carboxylic acid (6.89 mg, 12.27 μmol, 17.05% yield, 96.51% purity) was obtained as a white solid.

LCMS: RT=2.239 min, MS cal.: 542.02, [M+H]⁺=542.1

HPLC: RT=11.413 min, purity: 96.51%

¹H NMR (400 MHz, MeOH-d4) δ=7.86-7.81 (m, 1H), 7.56 (t, J=7.6 Hz, 1H), 7.51-7.42 (m, 2H), 7.24-7.12 (m, 2H), 6.81 (d, J=7.2 Hz, 1H), 6.62 (d, J=8.0 Hz, 1H), 5.39 (s, 2H), 3.98 (s, 3H), 3.90 (s, 2H), 3.03 (br d, J=11.6 Hz, 2H), 2.69-2.58 (m, 1H), 2.39-2.28 (m, 2H), 1.90-1.82 (m, 4H)

### Example A131—Preparation of Compound 208 and Compound 206. 2-((4-(6-((4-Chloro-2-fluorobenzyl)oxy)pyridin-2-yl)piperidin-1-yl)methyl)-4-(difluoromethoxy)-1-(fluoromethyl)-1H-benzo[d]imidazole-6-carboxylic acid (Compound 208) (11) & 2-((4-(6-((4-Chloro-2-fluorobenzyl)oxy)pyridin-2-yl)piperidin-1-yl)methyl)-7-(difluoromethoxy)-1-(fluoromethyl)-1H-benzo[d]imidazole-5-carboxylic acid (Compound 206) (11A)

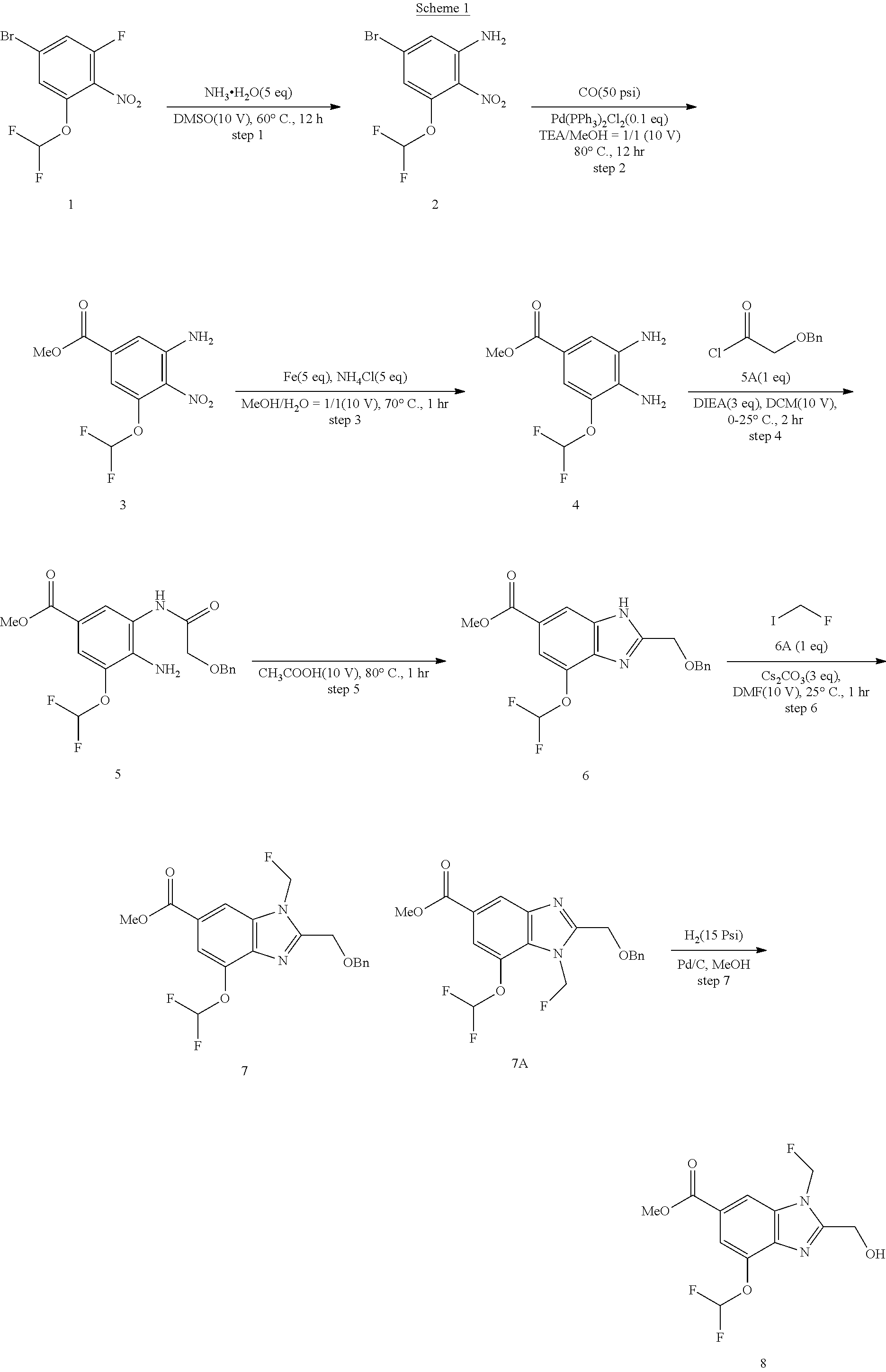

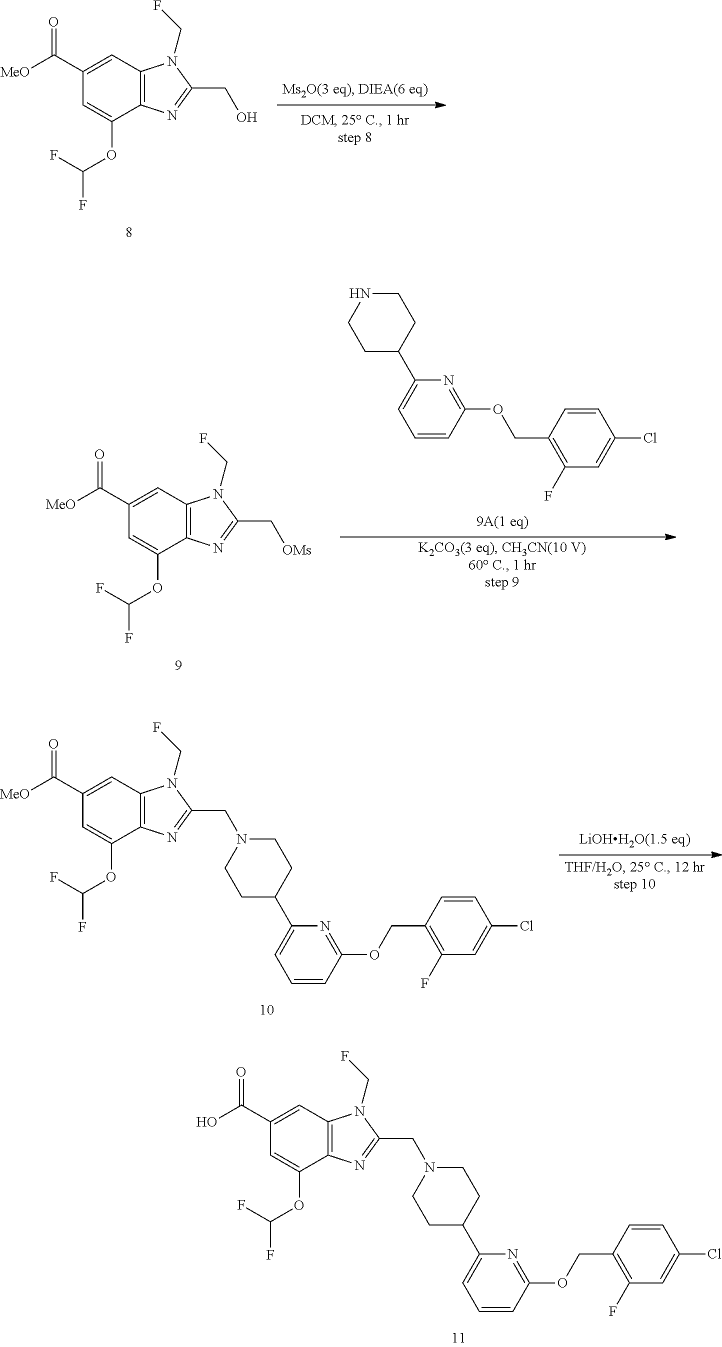

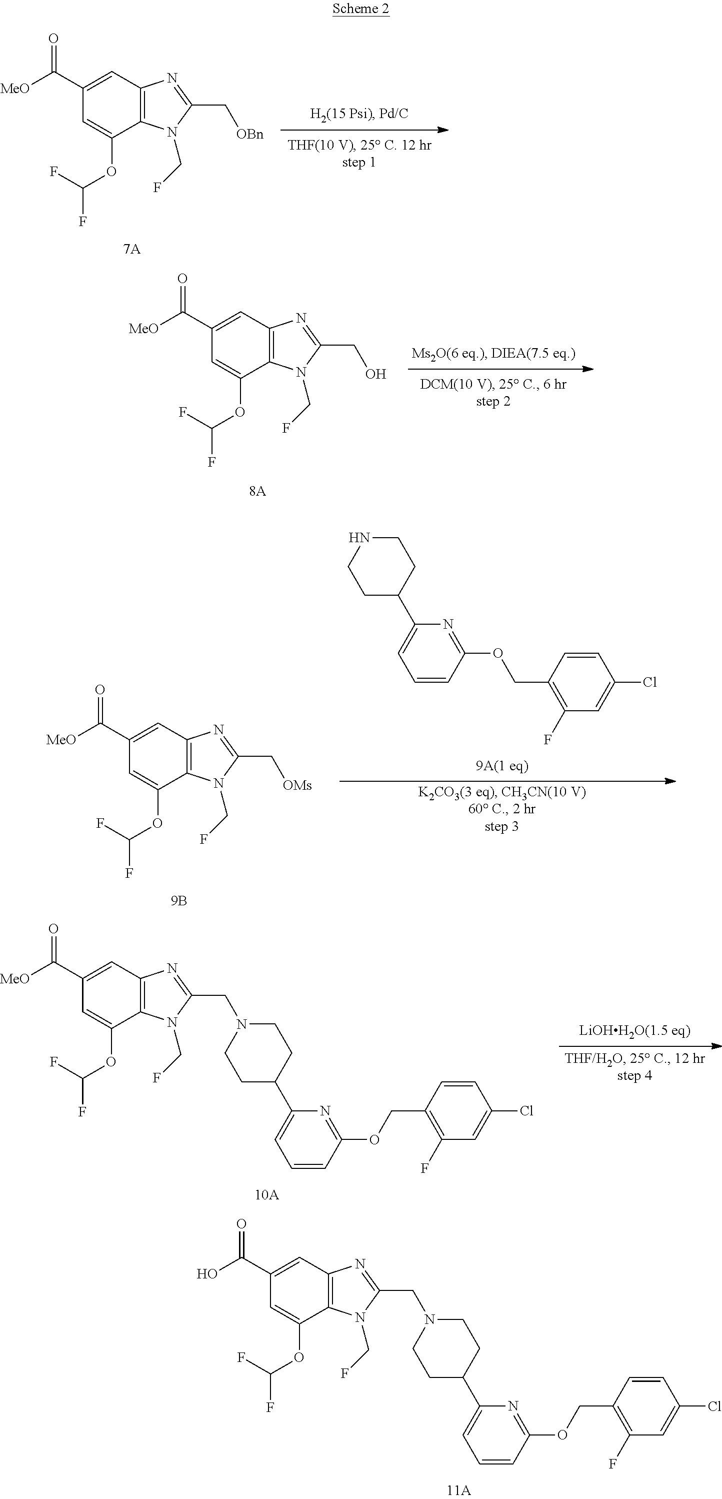

### General Procedure for Preparation of Scheme 1, Intermediate 2:

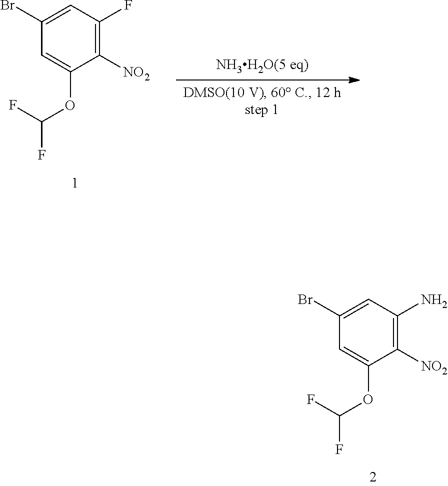 [SMILES: O=[N+]([O-])c1c(F)cc(Br)cc1OC(F)F ; "DMSO(10 V), 60° C., 12 hstep 1" ; "NH3·H2O(5 eq)" ; Nc1cc(Br)cc(OC(F)F)c1[N+](=O)[O-]]

A mixture of 1 (5 g, 17.48 mmol, 1 eq), NH₃·H₂O (12.25 g, 87.41 mmol, 13.47 mL, 25% purity, 5 eq) in DMSO (50 mL) was degassed and purged with N₂ for 3 times, and then the mixture was stirred at 60° C. for 12 hr under N₂ atmosphere. LC-MS (ET43536-1560-P1A1 product Rt=1.157 min) showed 1 was consumed completely and one main peak with desired mass was detected. The reaction mixture was diluted with H₂O 100 mL and extracted with EtOAc 300 mL (100 mL*3). The combined organic layers were washed with brine 50 mL, dried over Na₂SO₄, filtered and concentrated under reduced pressure to give a residue. 2 (4.6 g, 16.25 mmol, 92.97% yield) was obtained as a yellow solid.

LCMS: Rt₁=1.157 min, MS cal.: 281.9, [M+H]⁺=219.0

¹H NMR (400 MHz, CHCl3-d) δ=6.89 (d, J=1.92 Hz, 1H) 6.77-6.71 (m, 1H) 6.55 (t, J=72.8 Hz 1H) 6.37 (s, 1H) 4.76-5.55 (m, 2H)

### General Procedure for Preparation of Scheme 1, Intermediate 3:

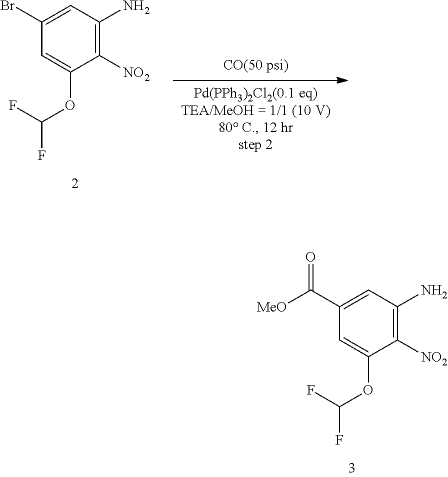

TEA (20 mL) and MeOH (20 mL) was charged to the 75 mL Hydrogenated bottle flask, then 2 (4.6 g, 16.25 mmol, 1 eq) Pd(PPh₃)₂Cl₂ (1.14 g, 1.63 mmol, 0.1 eq) was added at 25° C. After the addition, purged with CO for 3 times, the mixture was stirred at 80° C. 50 psi for 12 hr. TLC (Petroleum ether:Ethyl acetate=3:1 Rf=0.33) indicated 2 was consumed completely and one new spot formed. The reaction was clean according to TLC. The suspension was filtered through diatomite and filter cake was washed with EtOAc 10 ml*3. The combined filtrate were diluted with H₂O 10 mL and extracted with EtOAc 30 mL (10 mL*3). The combined organic layers were washed with brine 10 mL, dried over Na₂SO₄, filtered and concentrated under reduced pressure to give a residue. The residue was purified by column chromatography (SiO₂, Petroleum ether/Ethyl acetate=100/1 to 1/1). 3 (1.34 g, 5.11 mmol, 31.45% yield) was obtained as a white solid.

1H NMR (400 MHz, CHCl3-d) δ=7.40 (d, J=1.52 Hz, 1H) 7.21 (d, J=1.08 Hz, 1H) 6.59 (t, J=72.8 Hz, 1H) 6.41 (s, 1H) 5.15 (br s, 2H) 3.95 (s, 3H)

### General Procedure for Preparation of Scheme 1, Intermediate 4:

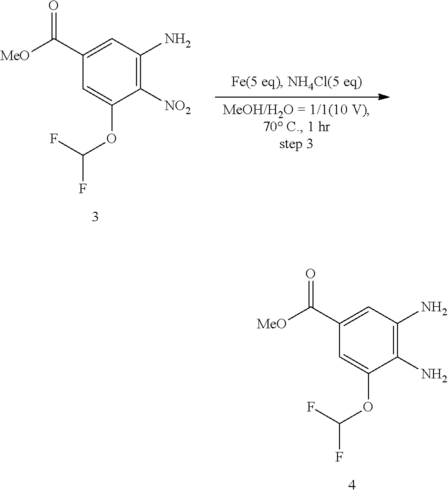

MeOH (6 mL) was charged to the 50 mL three-necked round bottom flask 3 (1.3 g, 4.96 mmol, 1 eq) was added at 20° C., H₂O (6 mL) was charged to the 50 mL three-necked round bottom flask NH₄Cl (1.33 g, 24.79 mmol, 5 eq) was added at 20° C., At 20° C. inner temperature Fe (1.38 g, 24.79 mmol, 5 eq) was added dropwise to the reaction mixture at 20° C., After the addition, the mixture was stirred at 70° C. for 1 h. LC-MS showed 3 was consumed completely and one main peak with desired mass was detected. The resulting product was dissolved in MeOH (6 mL) and filtered to remove the insoluble. The reaction mixture was added to 30 mL (H₂O) at 20° C., the mixture was extracted by EtOAc (30 mL). The organic phase was combined and washed by H₂O (30 mL), brine (30 mL), dried with Na₂SO₄, filtered, the organic phase was concentrated under reduced pressure at 40° C. to give a residue. The residue was purified by column chromatography (SiO₂, Petroleum ether/Ethyl acetate=100/1 to 5/1). 4 (1 g, 4.31 mmol, 86.86% yield) was obtained as a white solid.

LCMS: Rt=0.343 min, MS cal.: 232.0, [M+H]⁺=233.1

1H NMR (400 MHz, MeOH-d4) δ=7.26 (d, J=1.72 Hz, 1H) 7.20 (d, J=0.72 Hz, 1H) 6.72 (t, J=74.4 Hz 1H) 3.82 (s, 3H)

### General Procedure for Preparation of Scheme 1, Intermediate 5:

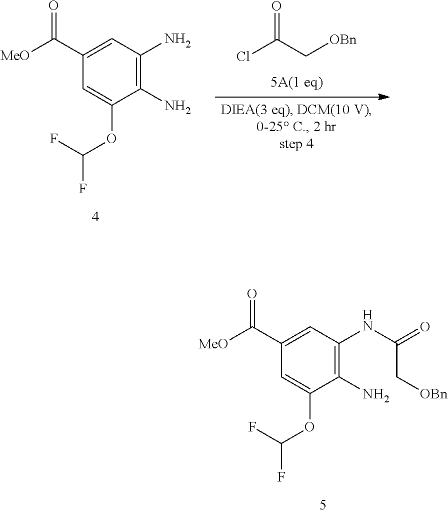

To a solution of 4 (1 g, 4.31 mmol, 1 eq) DIEA (1.67 g, 12.92 mmol, 2.25 mL, 3 eq) in DCM (10 mL) at 25° C. 5A (795.14 mg, 4.31 mmol, 668.75 μL, 1 eq) added to the mixture, The mixture was stirred at 25° C. for 2 hr. LC-MS (product Rt=0.476 min) showed 4 was consumed completely and one main peak with desired mass was detected. The reaction mixture was diluted with H₂O 50 mL and extracted with EtOAc 150 mL (50 mL*3). The combined organic layers were washed with brine 30 mL, dried over Na2so4, filtered and concentrated under reduced pressure to give a residue. 5 (1.6 g, crude) was obtained as a white solid.

LCMS: Rt₁=0.476 min, MS cal.: 380.1, [M+H]⁺=381.1

### General Procedure for Preparation of Scheme 1, Intermediate 6:

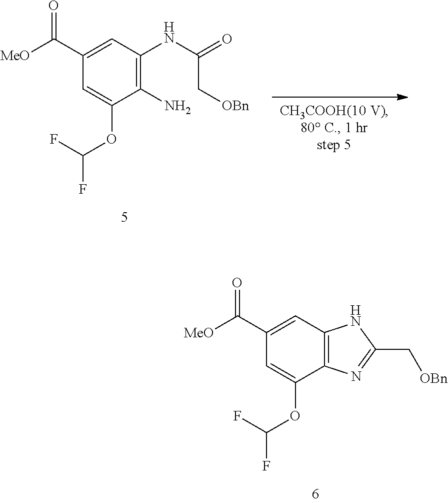 [SMILES: COC(=O)c1cc(NC(=O)COCc2ccccc2)c(N)c(OC(F)F)c1 ; "CH3COOH(10 V),80° C., 1 hrstep 5" ; COC(=O)c1cc(OC(F)F)c2nc(COCc3ccccc3)[nH]c2c1]

A mixture of 5 (1.6 g, 4.21 mmol, 1 eq) in CH₃COOH (16 mL) was degassed and purged with N₂ for 3 times, and then the mixture was stirred at 80° C. for 1 hr under N₂ atmosphere. LC-MS (product Rt=0.486 min) showed 5 was consumed completely and one main peak with desired mass was detected. The reaction mixture was diluted with NaHCO₃10 mL and extracted with EtOAc 30 mL (10 mL*3). The combined organic layers were washed with brine 10 mL dried over Na₂SO₄, filtered and concentrated under reduced pressure to give a residue. The residue was purified by column chromatography (SiO₂, Petroleum ether/Ethyl acetate=100/1 to 3/1). 6 (1 g, 2.76 mmol, 65.61% yield) was obtained as a white solid.

LCMS: Rt=0.486 min, MS cal.: 362.1, [M+H]⁺=363.0

### General Procedure for Preparation of Scheme 1, Intermediate 7:

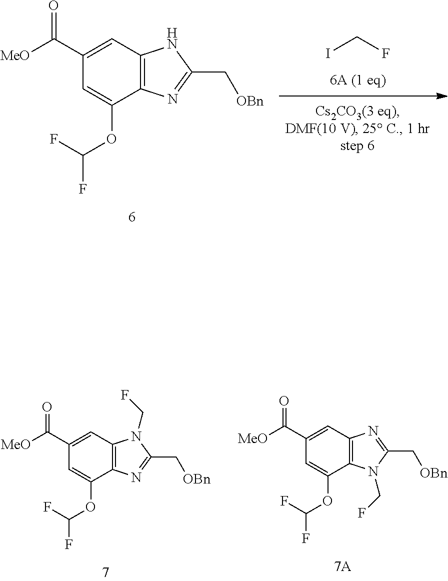

A mixture of 6A (441.39 mg, 2.76 mmol, 1 eq), 6 (1 g, 2.76 mmol, 1 eq), Cs₂CO₃ (2.70 g, 8.28 mmol, 3 eq), in DMF (10 mL) was degassed and purged with N₂ for 3 times, and then the mixture was stirred at 25° C. for 1 hr under N₂ atmosphere. LC-MS (Rt=0.559 min) showed 6 was consumed completely and one main peak with desired mass was detected. The reaction mixture was diluted with H₂O 20 mL and extracted with EtOAc 60 mL (20 mL*3). The combined organic layers were washed with brine 20 mL, dried over Na₂SO₄, filtered and concentrated under reduced pressure to give a residue. The residue was purified by column chromatography (SiO₂, Petroleum ether/Ethyl acetate=100/1 to 10/1). 7 (350 mg, 887.55 μmol, 32.16% yield) was obtained as a white solid. 7A (660 mg, crude) was obtained as a white solid.

LCMS: Rt₁=0.558 min, MS cal.: 394.1, [M+H]⁺=395.1

1H NMR (400 MHz, MeOH-d4) δ=8.20 (s, 1H) 7.72 (s, 1H) 7.40-7.35 (m, 2H) 7.34 (t, J=74 Hz, 1H) 7.33 (d, J=6.92 Hz, 3H) 7.31-7.24 (m, 1H) 7.15 (s, 1H) 6.44 (d, J=51.6 Hz, 2H) 4.94 (s, 2H) 4.63 (s, 2H) 3.96 (s, 3H)

### General Procedure for Preparation of Scheme 1, Intermediate 8:

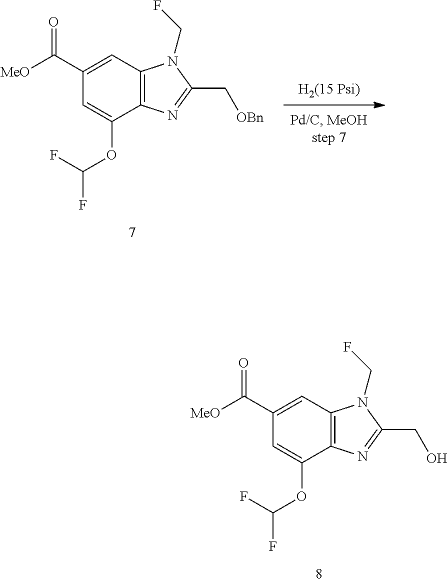 [SMILES: COC(=O)c1cc(OC(F)F)c2nc(COCc3ccccc3)n(CF)c2c1 ; COC(=O)c1cc(OC(F)F)c2nc(CO)n(CF)c2c1]

THF (2 mL) was charged to the 75 mL Hydrogenated bottle flask, then Pd/C (100 mg) and 7 (200 mg, 507.17 μmol, 1 eq) was added at 25° C. After the addition, purged with H₂ for 3 times the mixture was stirred at 50° C. 15 psi for 2 hr. LC-MS showed 7 was consumed completely and one main peak with desired mass was detected. The suspension was filtered through diatomite and filter cake was washed with EtOAc 10 ml*3. The combined organic layers were reduced pressure to give a residue. 8 (150 mg, 493.06 μmol, 97.22% yield) was obtained as a white solid.

LCMS: Rt=0.983 min, MS cal. 304.0, [M+H]⁺=304.9

1H NMR (400 MHz, MeOH-d4) δ=8.22 (s, 1H) 7.73 (s, 1H) 7.35 (t, J=74 Hz, 1H) 6.51 (d, J=48.8 Hz, 2H) 4.96 (s, 2H) 4.01-3.98 (m, 4H)

### General Procedure for Preparation of Scheme 1, Intermediate 9:

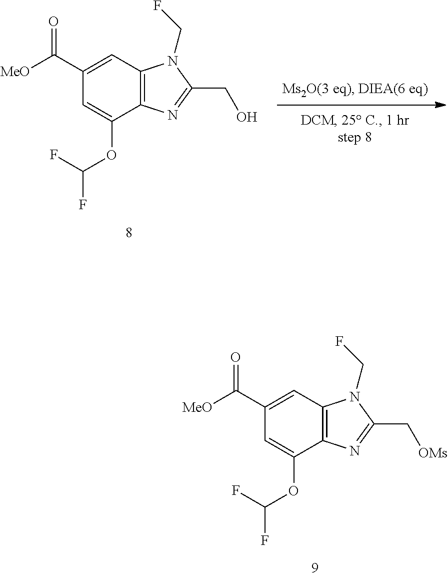 [SMILES: COC(=O)c1cc(OC(F)F)c2nc(CO)n(CF)c2c1 ; COC(=O)c1cc(OC(F)F)c2nc(COS(C)(=O)=O)n(CF)c2c1]

A mixture of 8 (100 mg, 328.71 μmol, 1 eq), DIEA (254.90 mg, 1.97 mmol, 343.53 μL, 6 eq), Ms₂O (171.78 mg, 986.12 μmol, 3 eq) in DCM (1 mL) was degassed and purged with N₂ for 3 times, and then the mixture was stirred at 25° C. for 1 hr under N₂ atmosphere. LC-MS (Rt=0.443 min) showed 8 was consumed completely and one main peak with desired mass was detected. The reaction mixture was diluted with H₂O 10 mL and extracted with EtOAc 30 mL (10 mL*3). The combined organic layers were washed with brine 10 mL, dried over Na₂SO₄, filtered, and concentrated under reduced pressure to give a residue. 9 (125 mg, crude) was obtained as a white solid.

LCMS: Rt=0.443 min, MS cal. 382.0, [M+H]⁺=383.0

### General Procedure for Preparation of Scheme 1, Intermediate 10:

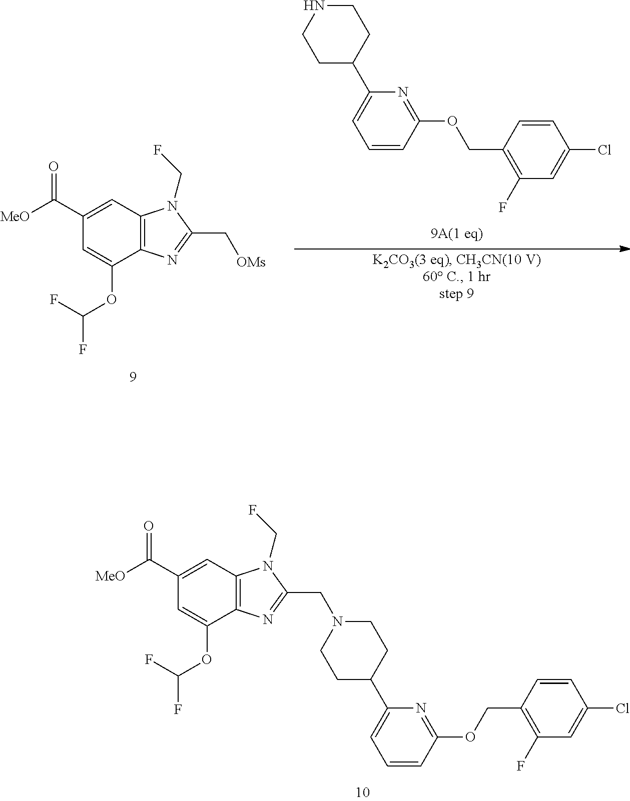 [SMILES: COC(=O)c1cc(OC(F)F)c2nc(COS(C)(=O)=O)n(CF)c2c1 ; Fc1cc(Cl)ccc1COc1cccc(C2CCNCC2)n1 ; "9A(1 eq)" ; "K2CO3(3 eq), CH3CN(10 V)60° C., 1 hrstep 9" ; COC(=O)c1cc(OC(F)F)c2nc(CN3CCC(c4cccc(OCc5ccc(Cl)cc5F)n4)CC3)n(CF)c2c1]

A mixture of 9A (104.88 mg, 326.96 μmol, 1 eq), 9 (125 mg, 326.96 μmol, 1 eq), K₂CO₃ (135.56 mg, 980.87 μmol, 3 eq), in CH₃CN (1 mL) was degassed and purged with N₂ for 3 times, and then the mixture was stirred at 60° C. for 1 hr under N₂ atmosphere. TLC (Petroleum ether:Ethyl acetate=1:1) indicated 9 was consumed completely and one new spot formed. The reaction mixture was diluted with H₂O 10 mL and extracted with EtOAc 30 mL (10 mL*3). The combined organic layers were washed with brine 10 mL, dried over Na₂SO₄, filtered and concentrated under reduced pressure to give a residue. The residue was purified by column chromatography (SiO₂, Petroleum ether/Ethyl acetate=100/1 to 1/1). 10 (120 mg, 197.70 μmol, 60.47% yield) was obtained as a white solid.

1H NMR (400 MHz, MeOH-d4) δ=8.24 (d, J=1.20 Hz, 1H) 7.74 (s, 1H) 7.54-7.59 (m, 1H) 7.53-7.47 (m, 1H) 7.33 (t, J=74 Hz, 1H) 7.23-7.16 (m, 2H) 7.14 (s, 1H) 6.82 (d, J=7.24 Hz, 1H) 6.70 (s, 1H) 6.70-9.57 (d, J=51.6 Hz, 2H) 6.57 (s, 1H) 5.40 (s, 2H) 4.00 (s, 2H) 3.97 (s, 3H) 3.22 (s, 1H) 3.00 (br d, J=11.08 Hz, 2H) 2.68-2.59 (m, 1H) 2.35-2.27 (m, 2H) 2.22 (s, 1H) 1.93-1.82 (m, 4H)

### General Procedure for Preparation of Scheme 1, Compound 208 (11):

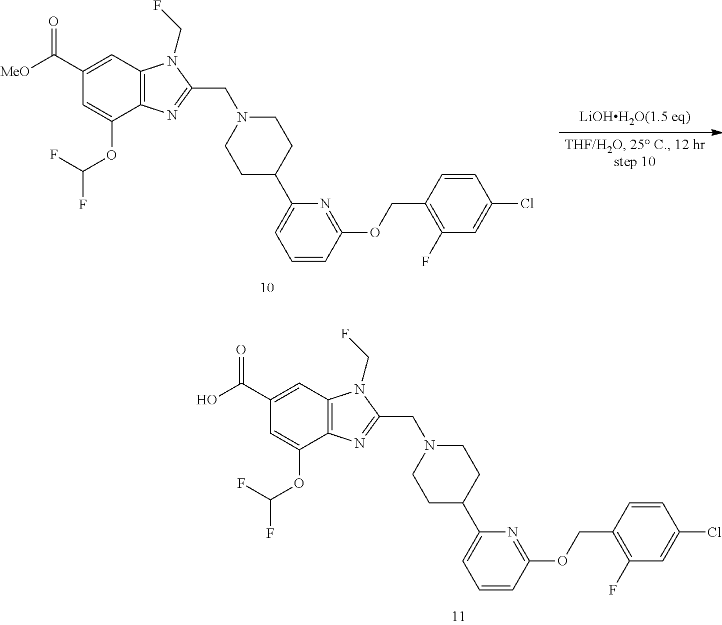 [SMILES: COC(=O)c1cc(OC(F)F)c2nc(CN3CCC(c4cccc(OCc5ccc(Cl)cc5F)n4)CC3)n(CF)c2c1 ; "LiOH·H2O(1.5 eq)" ; "THF/H2O, 25° C., 12 hrstep 10" ; O=C(O)c1cc(OC(F)F)c2nc(CN3CCC(c4cccc(OCc5ccc(Cl)cc5F)n4)CC3)n(CF)c2c1]

A mixture of 10 (100 mg, 164.75 μmol, 1 eq), LiOH·H₂O (10.37 mg, 247.12 μmol, 1.5 eq), in THF (0.7 mL) H₂O (0.3 mL) was degassed and purged with N₂ for 3 times, and then the mixture was stirred at 25° C. for 16 hr under N₂ atmosphere. LC-MS (ET43536-1601-P1b1 product Rt=0.954 min) showed 10 was consumed completely and one main peak with desired mass was detected. The residue was purified directly. The residue was purified by prep-HPLC (neutral condition column: Waters Xbridge Prep OBD C18 150*40 mm*10 um; mobile phase: [H₂O (10 mM NH₄HCO₃)-ACN]; gradient: 30%-70% B over 8.0 min). 2-((4-(6-((4-Chloro-2-fluorobenzyl)oxy)pyridin-2-yl)piperidin-1-yl)methyl)-4-(difluoromethoxy)-1-(fluoromethyl)-1H-benzo[d]imidazole-6-carboxylic acid (29.71 mg, 50.10 μmol, 30.41% yield) was obtained as a grey white solid.

LCMS: Rt₁=2.900 min, MS cal.: 592.1, [M+H]⁺=593.2

H NMR (400 MHz, MeOH-d4) δ=8.20 (s, 1H) 7.75 (s, 1H) 7.57 (t, J=7.80 Hz, 1H) 7.52-7.46 (m, 1H) 7.30 (t, J=74 Hz, 1H) 7.23-7.14 (m, 2H) 7.11 (s, 1H) 6.82 (d, J=7.28 Hz, 1H) 6.68-6.55 (d, J=52 Hz, 2H) 6.62 (d, J=8.12 Hz, 1H) 5.40 (s, 2H) 4.03 (s, 2H) 3.04 (br d, J=11.08 Hz, 2H) 2.71-2.57 (m, 1H) 2.42-2.29 (m, 2H) 1.95-1.81 (m, 4H)

### General Procedure for Preparation of Scheme 2, Intermediate 8A:

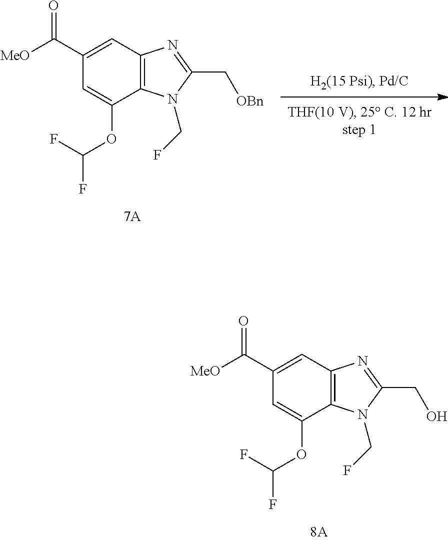 [SMILES: COC(=O)c1cc(OC(F)F)c2c(c1)nc(COCc1ccccc1)n2CF ; COC(=O)c1cc(OC(F)F)c2c(c1)nc(CO)n2CF]

THF (1.3 mL) was charged to the 50 mL one-necked round bottom flask, then 7A (0.13 g, 329.66 μmol, 1 eq) was added to the mixture at 25° C. At 25° C. (inner temperature), Pd/C (56.13 mg) was added dropwise to the reaction mixture at 20° C. After the addition, the mixture was stirred at 50° C. for 12 hr. LCMS showed 7A was consumed completely and one main peak with desired mass was detected. The reaction mixture was filtered, and the filter cake was washed with MeOH. The combined filtrate was concentrated to dryness to give product. 8A (0.1 g, crude) was obtained as a white solid.

LCMS: RT=0.959 min, MS cal.: 304.1/305.1, [M+1]⁺=305.0/305.9

¹HNMR (400 MHz, CHCl3-d) δ=8.29-8.26 (m, 1H), 7.78 (s, 1H), 6.73 (t, J=74.4 Hz 1H), 6.483 (d, J=52 Hz, 2H), 5.07-5.03 (m, 2H), 4.00-3.96 (m, 3H)

### General Procedure for Preparation of Scheme 2, Intermediate 9A:

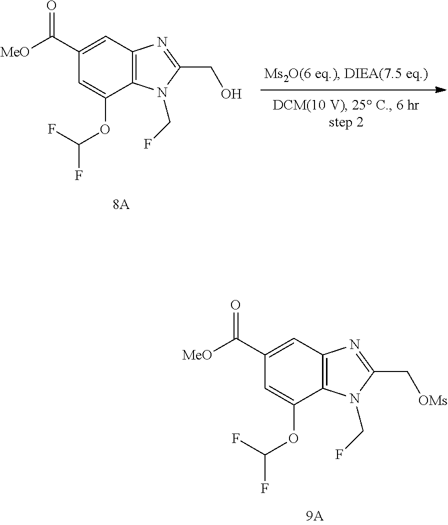 [SMILES: COC(=O)c1cc(OC(F)F)c2c(c1)nc(CO)n2CF ; COC(=O)c1cc(OC(F)F)c2c(c1)nc(COS(C)(=O)=O)n2CF]

To a solution of 8A (0.1 g, 328.71 μmol, 1 eq) in DCM (1 mL) was added DIEA (254.90 mg, 1.97 mmol, 343.53 μL, 6 eq) and Ms₂O (171.78 mg, 986.12 μmol, 3 eq). The mixture was stirred at 25° C. for 4 hr. After monitoring, the mixture was added Ms₂O (57.26 mg, 328.71 μmol, 1 eq) and DIEA (63.72 mg, 493.06 μmol, 85.88 μL, 1.5 eq). The mixture was stirred at 25° C. for 2 hr. LCMS showed 8A was consumed completely and one main peak with desired mass was detected. 9A (125 mg, crude) was obtained as a brown oil.

LCMS: RT=0.437 min, MS cal.: 382.0/383.0, [M+1]⁺=383.2/384.0

### General Procedure for Preparation of Scheme 2, Intermediate 10A:

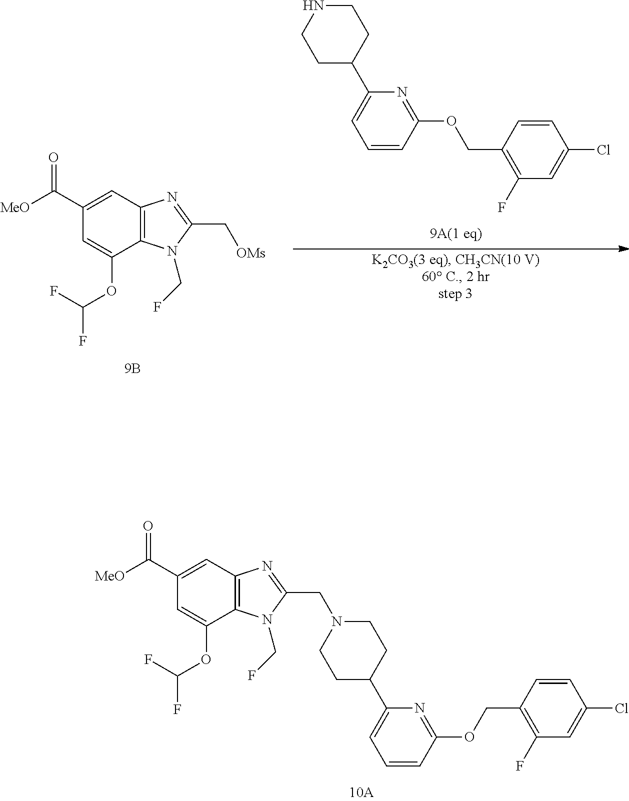 [SMILES: COC(=O)c1cc(OC(F)F)c2c(c1)nc(COS(C)(=O)=O)n2CF ; Fc1cc(Cl)ccc1COc1cccc(C2CCNCC2)n1 ; "9A(1 eq)" ; "K2CO3(3 eq), CH3CN(10 V)60° C., 2 hrstep 3" ; COC(=O)c1cc(OC(F)F)c2c(c1)nc(CN1CCC(c3cccc(OCc4ccc(Cl)cc4F)n3)CC1)n2CF]

To a solution of 9B (0.125 g, 326.96 μmol, 1 eq) in CH₃CN (2 mL) was added K₂CO₃ (135.57 mg, 980.87 μmol, 3 eq) and 9A (104.88 mg, 326.96 μmol, 1 eq). The mixture was stirred at 60° C. for 2 hr. LCMS showed 9A was consumed completely and one main peak with desired mass was detected. The reaction mixture was diluted with H₂O 5 mL and extracted with EtOAc 10 mL (5 mL*2). The combined organic layers were washed with brine 5 mL, dried over Na₂SO₄, filtered and concentrated under reduced pressure to give a residue. The residue was purified by column chromatography (SiO₂, PE/EtOAc=1/0 to 1/1). 10A (50 mg, 82.37 μmol, 25.19% yield) was obtained as a yellow oil.

LCMS: RT=1.759 min, MS cal.: 606.2/608.2, [M+1]⁺=607.1/609.1

### General Procedure for Preparation of Scheme 2, Compound 206 (11A):

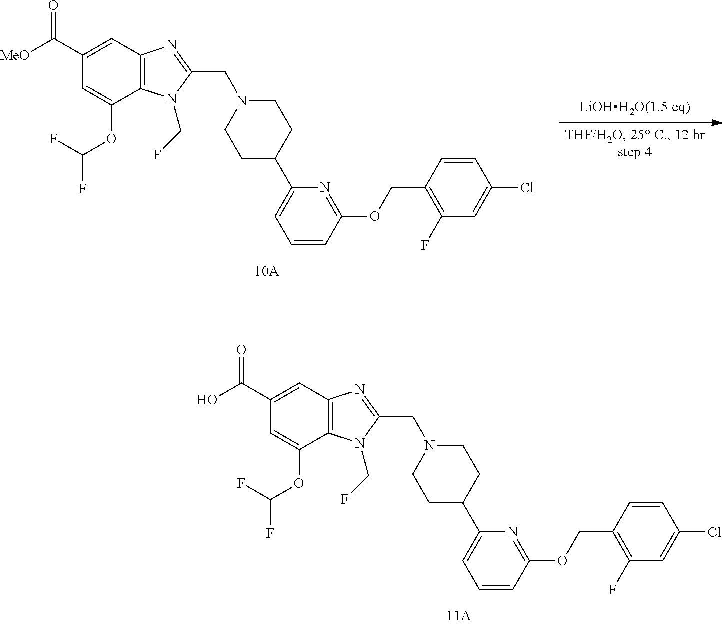 [SMILES: COC(=O)c1cc(OC(F)F)c2c(c1)nc(CN1CCC(c3cccc(OCc4ccc(Cl)cc4F)n3)CC1)n2CF ; "LiOH·H2O(1.5 eq)" ; "THF/H2O, 25° C., 12 hrstep 4" ; O=C(O)c1cc(OC(F)F)c2c(c1)nc(CN1CCC(c3cccc(OCc4ccc(Cl)cc4F)n3)CC1)n2CF]

To a solution of 10A (45 mg, 74.14 μmol, 1 eq) in THF (0.7 mL) and H₂O (0.3 mL) was added LiOH·H₂O (4.67 mg, 111.20 μmol, 1.5 eq). The mixture was stirred at 25° C. for 12 hr. LCMS showed 10A was consumed completely and one main peak with desired mass was detected. The mixture was added ACN 0.5 mL. The residue was purified by prep-HPLC (neutral condition: column: Waters Xbridge Prep OBD C18 150*40 mm*10 um; mobile phase: [H₂O (10 mM NH₄HCO₃)-ACN]; gradient: 15%-65% B over 8.0 min). 2-((4-(6-((4-Chloro-2-fluorobenzyl)oxy)pyridin-2-yl)piperidin-1-yl)methyl)-7-(difluoromethoxy)-1-(fluoromethyl)-1H-benzo[d]imidazole-5-carboxylic acid (23.50 mg, 39.23 μmol, 52.92% yield) was obtained as a white solid.

LCMS: RT=1.277 min, MS cal.: 592.2/594.1, [M+H]⁺=593.1/595.1

LCMS: RT=2.920 min, MS cal.: 592.2/594.1, [M+H]⁺=593.2/595.2

HPLC: RT=12.285 min

¹H NMR (400 MHz, DMSO-d₆) δ=8.23 (s, 1H), 7.84 (s, 1H), 7.58 (t, J=7.8 Hz, 1H), 7.54-7.47 (m, 1H), 7.23-7.16 (m, 2H), 7.12 (t, J=72.8 Hz, 1H), 6.80 (s, 1H), 6.73-6.60 (d, J=51.6 Hz, 2H), 6.65-6.62 (m, 1H), 5.41 (s, 2H), 4.01 (s, 2H), 3.08-3.01 (m, 2H), 2.72-2.60 (m, 1H), 2.41-2.28 (m, 2H), 1.97-1.84 (m, 4H)

### Example A132—Preparation of Compound 209. 2-((4-(6-((4-Chloro-2-fluorobenzyl)oxy)pyridin-2-yl)piperidin-1-yl)methyl)-1-(fluoromethyl)-4-methoxy-1H-benzo[d]imidazole-6-carboxylic acid (Compound 209) (9)

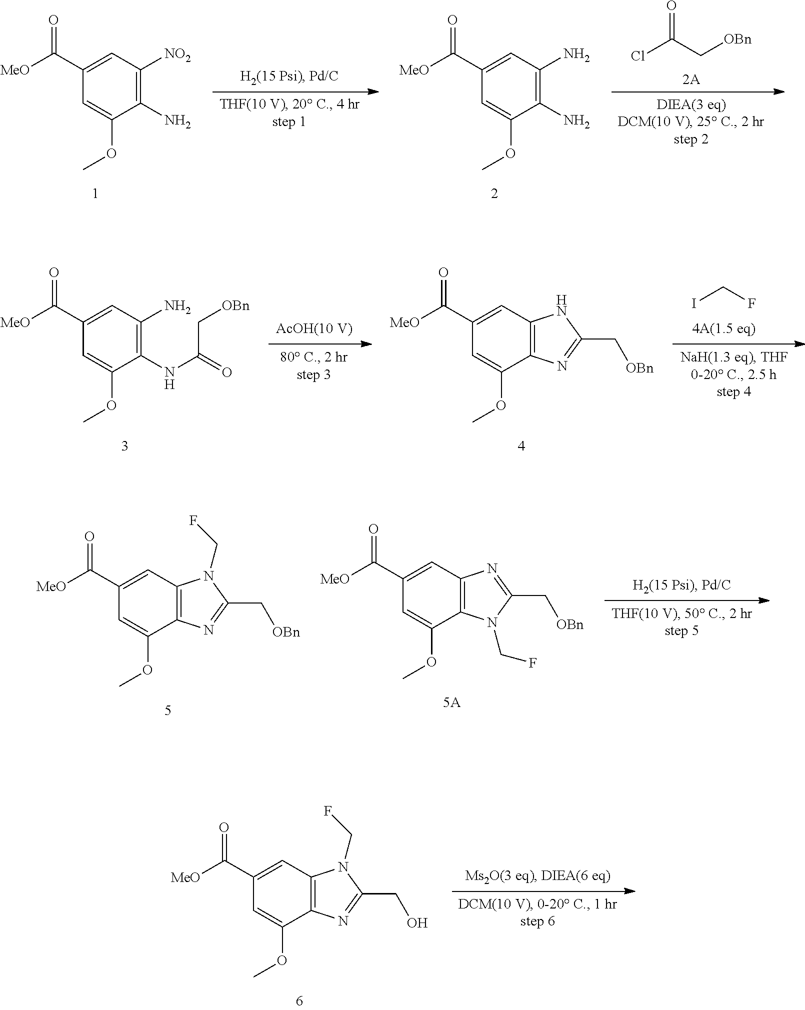

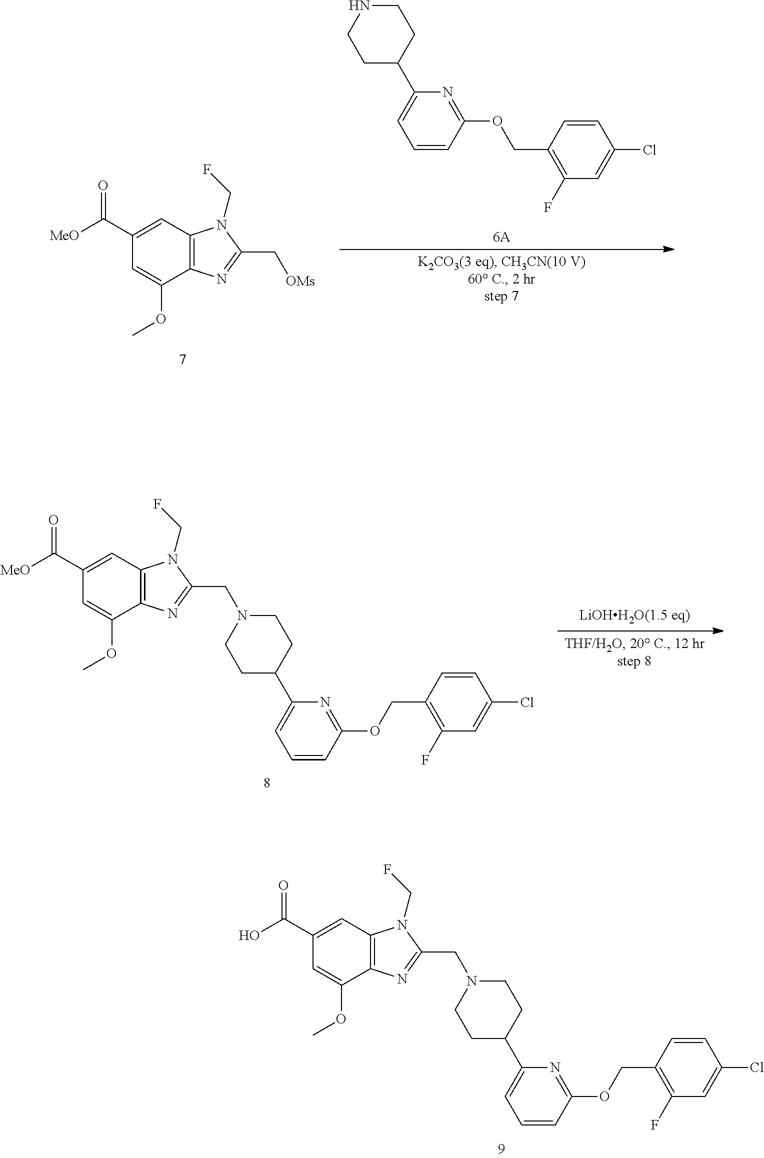

### General Procedure for Preparation of Intermediate 2:

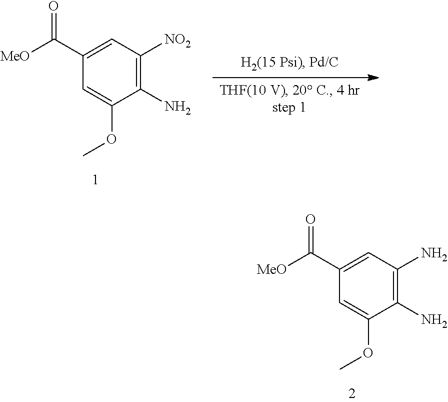 [SMILES: COC(=O)c1cc(OC)c(N)c([N+](=O)[O-])c1 ; "H2(15 Psi), Pd/C" ; "THF(10 V), 20° C., 4 hrstep 1" ; COC(=O)c1cc(N)c(N)c(OC)c1]

THF (20 mL) was charged to the round bottom flask, then 1 (1.5 g, 6.63 mmol, 1 eq) was added to the mixture at 20° C. At 20° C., Pd/C (400 mg) under N₂ atmosphere. The suspension was degassed and purged with H₂ for 3 times. The mixture was stirred under H₂ (15 Psi) at 20° C. for 4 hr. TLC (Petroleum ether:Ethyl acetate=2:1, Rf=0.21) indicated 1 was consumed completely and one new spot formed. The mixture was filtered through a Celite pad, and filter cake was washed with THF (10 mL*3). The combined filtrates were concentrated to dryness to give product. The residue was purified by column chromatography (SiO₂, Petroleum ether/Ethyl acetate=10/1 to 1/1). 2 (1 g, 5.10 mmol, 76.85% yield) was obtained as a white solid.

¹H NMR (400 MHz, DMSO-d6) δ=6.97 (d, J=1.6 Hz, 1H), 6.83 (d, J=1.6 Hz, 1H), 4.81 (br s, 2H), 4.74 (br s, 2H), 3.76 (s, 3H), 3.73 (s, 3H)

### General Procedure for Preparation of Intermediate 3:

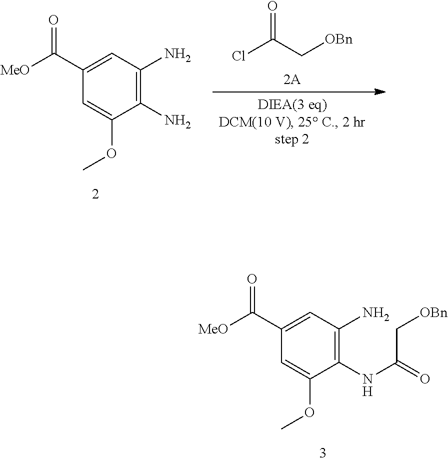

To a solution of 2 (800 mg, 4.08 mmol, 1 eq) and DIEA (1.58 g, 12.23 mmol, 2.13 mL, 3 eq) in DCM (10 mL) was added 2A (752.77 mg, 4.08 mmol, 633.11 μL, 1 eq) in DCM (2 mL). The mixture was stirred at 25° C. for 2 hr. LC-MS (showed ˜14% of 2 remained. Several new peaks were shown on LC-MS and ˜68% of desired was detected. The residue was diluted with H₂O 20 mL and extracted with DCM 60 mL (20 mL*3). The combined organic layers were washed with brine 10 mL, dried over Na₂SO₄, filtered, and concentrated under reduced pressure to give a residue. 3 (1.4 g, crude) was obtained as yellow oil.

LCMS: RT=1.467 min, MS cal.: 344.1, [M+H]⁺=345.1

### General Procedure for Preparation of Intermediate 4:

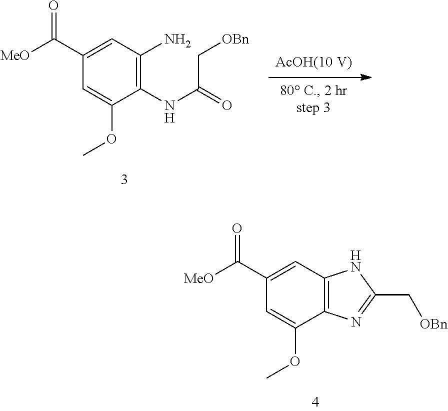 [SMILES: COC(=O)c1cc(N)c(NC(=O)COCc2ccccc2)c(OC)c1 ; "80° C., 2 hrstep 3" ; "AcOH(10 V)" ; COC(=O)c1cc(OC)c2nc(COCc3ccccc3)[nH]c2c1]

To a solution of 3 (1.2 g, 2.37 mmol, 1 eq) in AcOH (10 mL) was stirred at 80° C. for 2 hr. LC-MS showed 3 was consumed completely and desired mass was detected. The reaction mixture was concentrated under reduced pressure to remove solvent. The residue was diluted with NaHCO₃30 mL and extracted with DCM 60 mL (20 mL*3). The combined organic layers were washed with brine 20 mL, dried over Na₂SO₄, filtered and concentrated under reduced pressure to give a residue. The residue was purified by column chromatography (SiO₂, Petroleum ether/Ethyl acetate=10/1 to 0/1). 4 (800 mg, crude) was obtained as a white solid.

¹H NMR (400 MHz, DMSO-d6) δ=7.93-7.69 (m, 1H), 7.48-7.18 (m, 6H), 4.73 (br s, 2H), 4.61 (s, 2H), 3.98 (s, 3H), 3.86 (s, 3H)

### General Procedure for Preparation of Intermediate 5&5A:

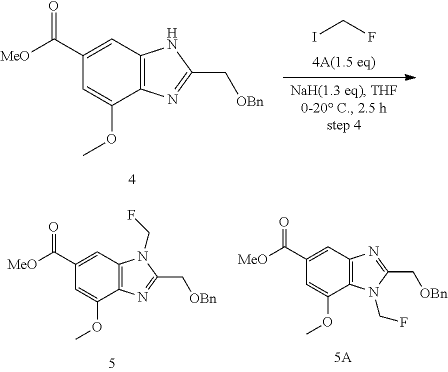

NaH (127.47 mg, 3.19 mmol, 60% purity, 1.3 eq) was added to a mixture of 4 (800 mg, 2.45 mmol, 1 eq) in THF (16 mL) at 0° C. The mixture was stirred for 30 min at 0° C. The 4A (588.07 mg, 3.68 mmol, 1.5 eq) was added and stirred for 2 h at 20° C. LC-MS showed ˜46% (Rt=1.633 min) & ˜33% (Rt=1.707 min) of desired was detected. The reaction mixture was quenched by addition NH₄Cl 30 mL at 20° C., and then extracted with EtOAc 60 (20 mL*3). The combined organic layers were washed with brine (10 mL), dried over Na₂SO₄, filtered, and concentrated under reduced pressure to give a residue. The residue was purified by column chromatography (SiO₂, Petroleum ether/Ethyl acetate=10/1 to 0/1). 5 (400 mg, 1.08 mmol, 44.08% yield, 96.8% purity) was obtained as a white solid. 5A (400 mg, 1.10 mmol, 45.03% yield, 98.9% purity) was obtained as a white solid.

LCMS: RT=1.633 min& 1.707 min, MS cal.: 358.1, [M+H]⁺=359.2

¹H NMR (400 MHz, DMSO-d6) δ=8.01 (d, J=1.2 Hz, 1H), 7.39-7.34 (m, 5H), 7.33-7.27 (m, 1H), 6.64-6.38 (d, J=52 Hz, 2H), 4.89 (s, 2H), 4.57 (s, 2H), 4.00 (s, 3H), 3.90 (s, 3H)

¹H NMR (400 MHz, DMSO-d6) δ=7.91 (d, J=1.2 Hz, 1H), 7.46 (d, J=0.8 Hz, 1H), 7.35 (d, J=4.2 Hz, 4H), 7.33-7.27 (m, 1H), 6.56-6.40 (d, J=52 Hz, 2H), 4.88 (s, 2H), 4.57 (s, 2H), 4.01 (s, 3H), 3.88 (s, 3H)

### General Procedure for Preparation of Intermediate 6:

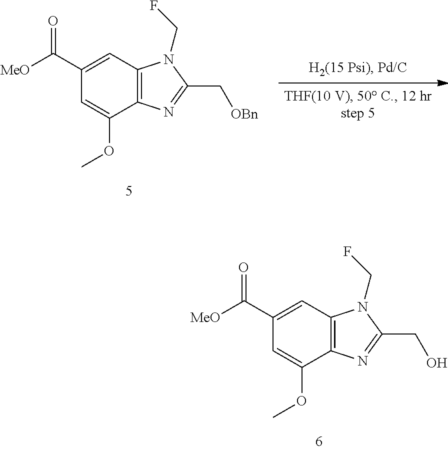 [SMILES: COC(=O)c1cc(OC)c2nc(COCc3ccccc3)n(CF)c2c1 ; "H2(15 Psi), Pd/C" ; "THF(10 V), 50° C., 12 hrstep 5" ; COC(=O)c1cc(OC)c2nc(CO)n(CF)c2c1]

THF (4 mL) was charged to the round bottom flask, 5 (400 mg, 1.12 mmol, 1 eq) was added to the mixture at 20° C. At 20° C., Pd/C (200 mg) under N₂ atmosphere. The suspension was degassed and purged with H₂ for 3 times at 20° C. The mixture was stirred under H₂ (15 Psi) at 50° C. for 12 hr. LC-MS showed 5 was consumed completely and desired mass was detected. The mixture was filtered through a Celite pad and washed by THF (5 mL*3) and the filtrate was concentrated to give the crude product. 6 (270 mg, 1.01 mmol, 90.18% yield) was obtained as a white solid.

¹H NMR (400 MHz, DMSO-d6) δ=7.98 (s, 1H), 7.36 (s, 1H), 6.58-6.45 (d, J=52.4 Hz, 2H), 5.85 (br t, J=5.2 Hz, 1H), 4.79 (br d, J=5.6 Hz, 2H), 3.99 (s, 3H), 3.92-3.86 (m, 3H)

### General Procedure for Preparation of Intermediate 7:

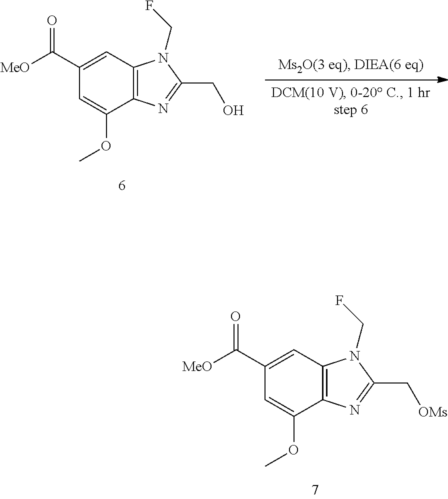 [SMILES: COC(=O)c1cc(OC)c2nc(CO)n(CF)c2c1 ; "DCM(10 V), 0-20° C., 1 hrstep 6" ; "Ms2O(3 eq), DIEA(6 eq)" ; COC(=O)c1cc(OC)c2nc(COS(C)(=O)=O)n(CF)c2c1]

To a solution of 6 (150 mg, 559.20 μmol, 1 eq) and DIEA (433.64 mg, 3.36 mmol, 584.41 μL, 6 eq) in DCM (2 mL) was added Ms₂O (292.23 mg, 1.68 mmol, 3 eq) at 0° C. The mixture was stirred at 20° C. for 1 hr. LCMS showed 6 was consumed completely and desired mass was detected. The residue was diluted with H₂O 10 mL and extracted with DCM 45 mL (15 mL*3). The combined organic layers were washed with brine 15 mL, dried over Na₂SO₄, filtered and concentrated under reduced pressure to give a residue. 7 (190 mg, crude) was obtained as yellow oil.

LCMS: RT=0.987 min, MS cal.: 346.0, [M+H]⁺=346.9

### General Procedure for Preparation of Intermediate 8:

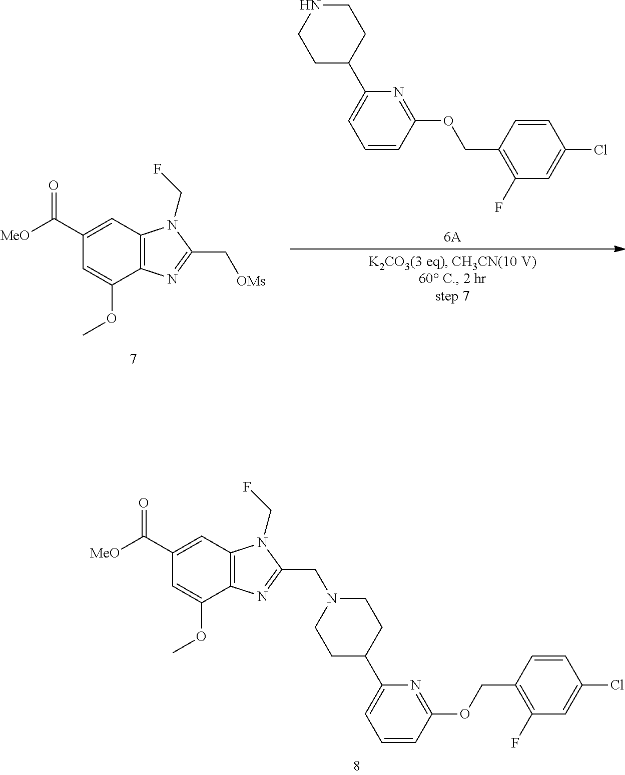 [SMILES: COC(=O)c1cc(OC)c2nc(COS(C)(=O)=O)n(CF)c2c1 ; Fc1cc(Cl)ccc1COc1cccc(C2CCNCC2)n1 ; "6A" ; "K2CO3(3 eq), CH3CN(10 V)60° C., 2 hrstep 7" ; COC(=O)c1cc(OC)c2nc(CN3CCC(c4cccc(OCc5ccc(Cl)cc5F)n4)CC3)n(CF)c2c1]

To a solution of 7 (190 mg, 548.61 μmol, 1 eq) in ACN (3 mL) was added K₂CO₃ (227.46 mg, 1.65 mmol, 3 eq) and 6A (175.99 mg, 548.61 μmol, 1 eq). The mixture was stirred at 60° C. for 2 hr. LC-MS showed 7 was consumed completely and desired mass was detected. The residue was diluted with H₂O 15 mL and extracted with EtOAc 45 mL (15 mL*3). The combined organic layers were washed with brine 15 mL, dried over Na₂SO₄, filtered and concentrated under reduced pressure to give a residue. The residue was purified by column chromatography (SiO₂, Petroleum ether/Ethyl acetate=10/1 to 0/1). 8 (170 mg, 297.72 μmol, 54.27% yield) was obtained as a white solid.

LCMS: RT=1.694 min, MS cal.: 570.1, [M+H]⁺=571.1

¹H NMR (400 MHz, MeOH-d4) δ=7.97 (d, J=1.2 Hz, 1H), 7.56 (t, J=7.6 Hz, 1H), 7.52-7.45 (m, 2H), 7.23-7.15 (m, 2H), 6.80 (d, J=7.2 Hz, 1H), 6.62 (d, J=8.4 Hz, 1H), 6.65-6.52 (d, J=52 Hz, 2H), 5.40 (s, 2H), 4.05 (s, 3H), 3.95 (s, 5H), 2.97 (br d, J=11.2 Hz, 2H), 2.68-2.57 (m, 1H), 2.34-2.23 (m, 2H), 1.93-1.81 (m, 4H)

### General Procedure for Preparation of Compound 209 (9):

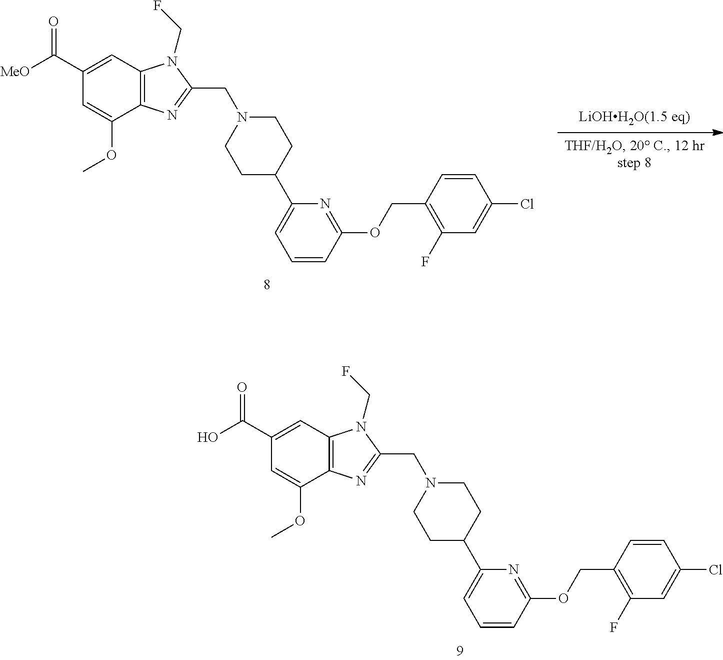 [SMILES: COC(=O)c1cc(OC)c2nc(CN3CCC(c4cccc(OCc5ccc(Cl)cc5F)n4)CC3)n(CF)c2c1 ; "LiOH·H2O(1.5 eq)" ; "THF/H2O, 20° C., 12 hrstep 8" ; COc1cc(C(=O)O)cc2c1nc(CN1CCC(c3cccc(OCc4ccc(Cl)cc4F)n3)CC1)n2CF]

To a solution of 8 (80 mg, 140.10 μmol, 1 eq) in THF (0.7 mL) was added LiOH·H₂O (8.82 mg, 210.15 μmol, 1.5 eq) in H₂O (0.3 mL). The mixture was stirred at 20° C. for 12 hr. LC-MS showed 8 was consumed completely and desired mass was detected. The reaction was added citric acid (0.5 M) to pH=7-8, then the mixture was purified directly. The residue was purified by prep-HPLC (neutral condition; column: Waters Xbridge BEH C18 100*30 mm*10 um; mobile phase: [H₂O (10 mM NH₄HCO₃)-ACN]; gradient: 30%-70% B over 8.0 min). 2-((4-(6-((4-Chloro-2-fluorobenzyl)oxy)pyridin-2-yl)piperidin-1-yl)methyl)-1-(fluoromethyl)-4-methoxy-1H-benzo[d]imidazole-6-carboxylic acid (28.8 mg, 51.53 μmol, 36.78% yield, 99.66% purity) was obtained as a white solid.

LCMS: RT=2.416 min, MS cal.: 556.1, [M+H]⁺=557.2

HPLC: RT=11.836 min, purity: 99.66%

¹H NMR (400 MHz, MeOH-d4) δ=7.87 (d, J=0.8 Hz, 1H), 7.59-7.47 (m, 3H), 7.24-7.15 (m, 2H), 6.81 (d, J=7.2 Hz, 1H), 6.62 (d, J=8.4 Hz, 1H), 6.63-6.50 (d, J=52.4 Hz, 2H), 5.40 (s, 2H), 4.04 (s, 3H), 3.94 (s, 2H), 2.99 (br d, J=11.2 Hz, 2H), 2.68-2.57 (m, 1H), 2.34-2.25 (m, 2H), 1.94-1.80 (m, 4H)

### Example A133—Preparation of Compound 210 and Compound 204. 2-((4-(6-((4-Chloro-2-fluorobenzyl)oxy)pyridin-2-yl)piperidin-1-yl)methyl)-7-(difluoromethoxy)-1H-benzo[d]imidazole-6-carboxylic acid (Compound 210) (5) & 2-((4-(6-((4-Chloro-2-fluorobenzyl)oxy)pyridin-2-yl)piperidin-1-yl)methyl)-7-hydroxy-1H-benzo[d]imidazole-6-carboxylic acid (Compound 204) (6)

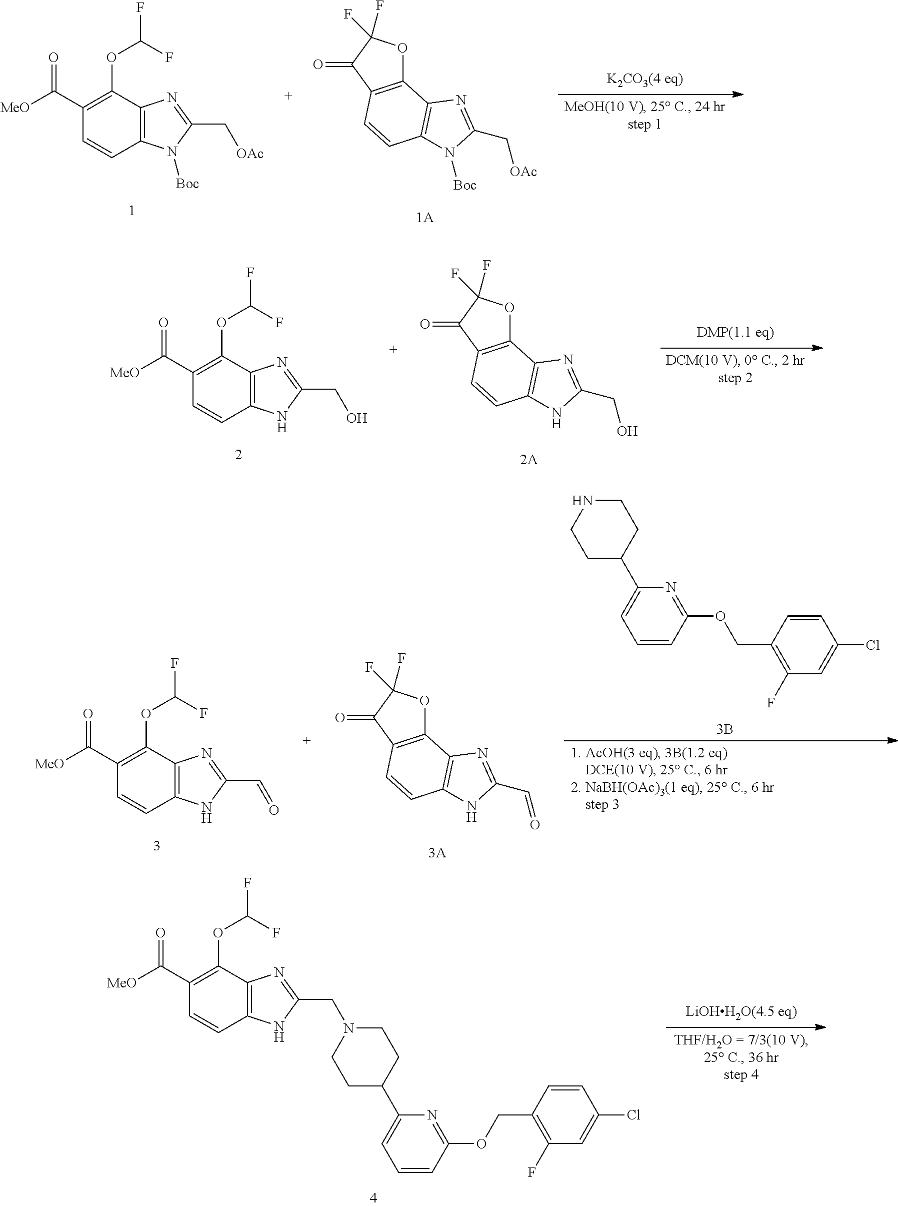

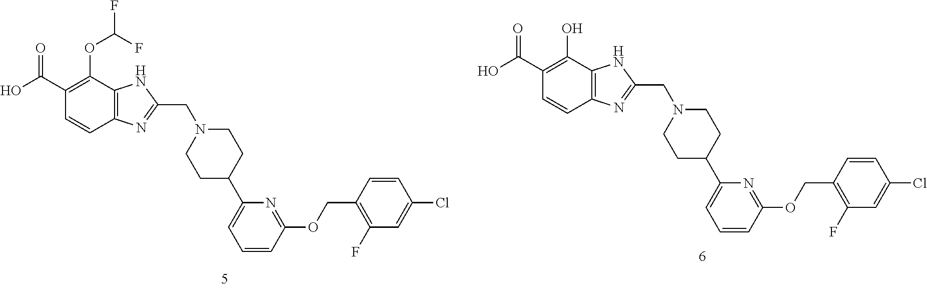 [SMILES: O=C(O)c1ccc2nc(CN3CCC(c4cccc(OCc5ccc(Cl)cc5F)n4)CC3)[nH]c2c1OC(F)F ; O=C(O)c1ccc2nc(CN3CCC(c4cccc(OCc5ccc(Cl)cc5F)n4)CC3)[nH]c2c1O]

### General Procedure for Preparation of Intermediate 2 & Intermediate 2A:

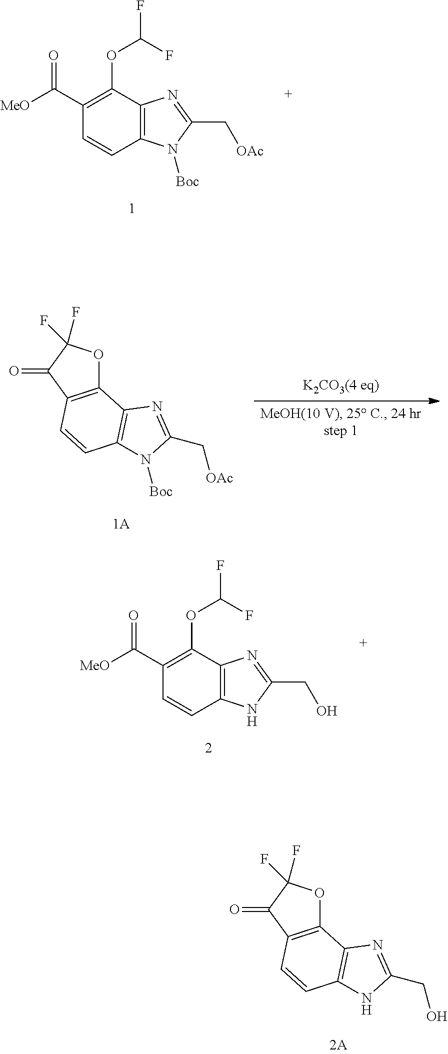 [SMILES: COC(=O)c1ccc2c(nc(COC(C)=O)n2C(=O)OC(C)(C)C)c1OC(F)F ; CC(=O)OCc1nc2c3c(ccc2n1C(=O)OC(C)(C)C)C(=O)C(F)(F)O3 ; "K2CO3(4 eq)" ; "MeOH(10 V), 25° C., 24 hrstep 1" ; COC(=O)c1ccc2[nH]c(CO)nc2c1OC(F)F ; O=C1c2ccc3[nH]c(CO)nc3c2OC1(F)F]

A mixture of 1 & 1A (1 g crude, ratio: 1/3 in HNMR), K₂CO₃ (333.55 mg, 2.41 mmol, 4 eq) in MeOH (10 mL) was degassed and purged with N₂ for 3 times, and then the mixture was stirred at 25° C. for 24 hr under N₂ atmosphere. LCMS (RT=0.855 min) showed 1 & 1A was consumed completely and one main peak with desired m/z was detected. The reaction mixture was filtered and concentrated under reduced pressure to give a residue. A mixture of 2 (640 mg, crude) was obtained as a white solid.

### General Procedure for Preparation of Intermediate 3 & Intermediate 3A:

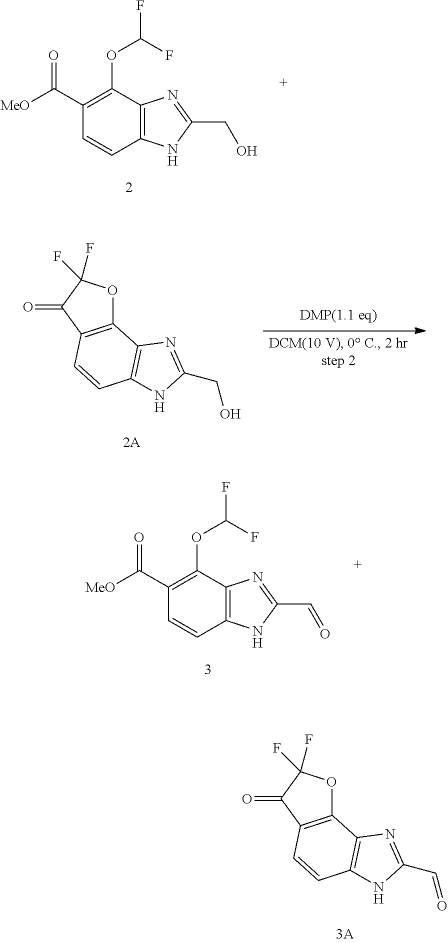 [SMILES: COC(=O)c1ccc2[nH]c(CO)nc2c1OC(F)F ; O=C1c2ccc3[nH]c(CO)nc3c2OC1(F)F ; "DCM(10 V), 0° C., 2 hrstep 2" ; "DMP(1.1 eq)" ; COC(=O)c1ccc2[nH]c(C=O)nc2c1OC(F)F ; O=Cc1nc2c3c(ccc2[nH]1)C(=O)C(F)(F)O3]

A mixture of 2 & 2A (640 mg crude), DMP (1.09 g, 646.57 μmol, 200.32 μL, 1.1 eq) in DCM (6.5 mL) was degassed and purged with N₂ for 3 times, and then the mixture was stirred at 0° C. for 2 hr under N₂ atmosphere. LCMS (RT=0.831 min) showed 2 & 2A was consumed completely and one main peak with desired mass was detected. The reaction mixture was quenched by addition Na₂S₃O₄ 10 mL at 25° C., and then diluted with H₂O 10 mL and extracted with DCM (10 mL*6). The combined organic layers were dried over Na₂SO₄, filtered, and concentrated under reduced pressure to give a residue. A mixture of 3 & 3A (600 mg, crude) was obtained as a white solid.

### General Procedure for Preparation of Intermediate 4:

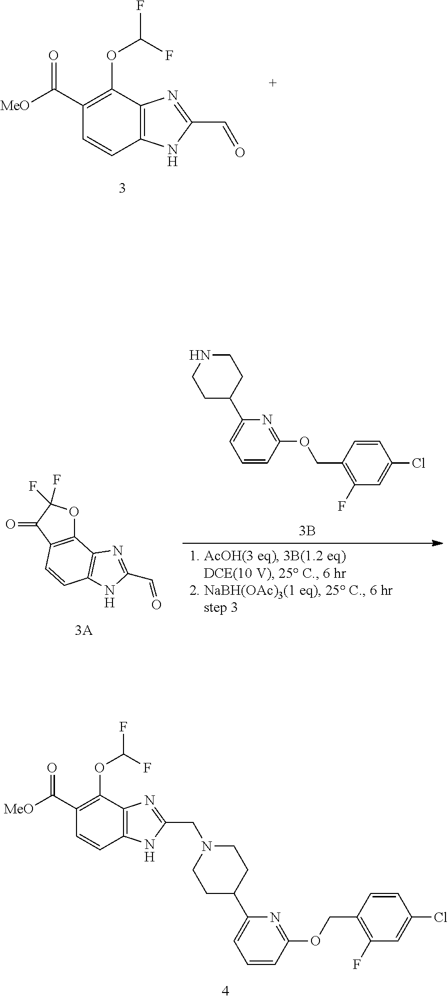

DCE (6 mL) was charged to the three-necked round bottom flask, then 3 & 3A (600 mg crude), 3B (854.84 mg, 666.20 μmol, 1.2 eq) was added to the mixture at 25° C. AcOH (400.08 mg, 1.67 mmol, 95.34 μL, 3 eq) was added dropwise to the reaction mixture at 25° C. And the mixture was stirred at 25° C. for 6 hr. NaBH(OAc)₃ (470.64 mg, 555.17 μmol, 1 eq) was added to the reaction mixture at 25° C. After the addition, the mixture was stirred at 25° C. for 6 hr. LCMS (RT=1.588 min) showed 3 & 3A was consumed completely and one main peak with desired mass was detected. The reaction mixture was diluted with H₂O 5 mL and extracted with DCM (5 mL*4). The combined organic layers were dried over Na₂SO₄, filtered and concentrated under reduced pressure to give a residue. The residue was purified by column chromatography (SiO₂, Petroleum ether/Ethyl acetate=100/1 to 0/1). The residue was repurified by prep-HPLC (column: Waters Xbridge Prep OBD C₁₈ 150*40 mm*10 um; mobile phase: [H₂O (10 mM NH₄HCO₃)-ACN]; gradient: 55%-75% B over 8.0 min). 4 (25 mg, 43.48 μmol, 7% yield) was obtained as a white solid.

LCMS: RT=1.590 min, MS cal.: 574.2, [M+H]⁺=575.0

¹H NMR (400 MHz, DMSO-d6) δ=13.07-12.77 (m, 1H), 7.69-7.61 (m, 2H), 7.57 (t, J=8.0 Hz, 1H), 7.50-7.39 (m, 2H), 7.31 (dd, J=1.6, 8.2 Hz, 1H), 6.88 (d, J=7.2 Hz, 1H), 6.68 (d, J=8.0 Hz, 1H), 5.37 (s, 2H), 3.84 (s, 3H), 3.80 (s, 2H), 2.96 (d, J=10.8 Hz, 2H), 2.68-2.54 (m, 1H), 2.30-2.15 (m, 2H), 1.81 (d, J=2.0 Hz, 4H)

### General Procedure for Preparation of Compound 210 (5) & Compound 204 (6):

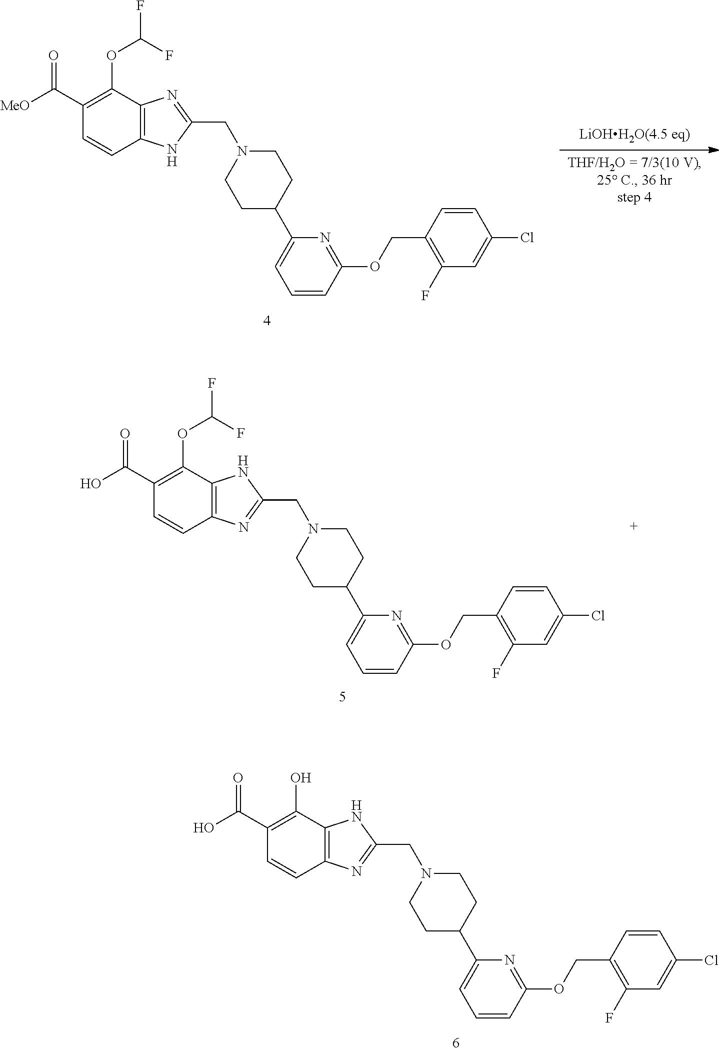

A mixture of 4 (20 mg, 34.78 μmol, 1 eq), LiOH·H₂O (3.65 mg, 86.96 μmol, 2.5 eq) in THF (0.7 mL) and H₂O (0.3 mL) was degassed and purged with N₂ for 3 times, and then the mixture was stirred at 25° C. for 12 hr under N₂ atmosphere. The mixture was added LiOH·H₂O (1.46 mg, 34.78 μmol, 1 eq), the mixture was stirred at 25° C. for 12 hr under N₂ atmosphere. The mixture was added LiOH·H₂O (1.46 mg, 34.78 μmol, 1 eq), the mixture was stirred at 25° C. for 12 hr under N₂ atmosphere. HPLC (2-((4-(6-((4-Chloro-2-fluorobenzyl)oxy)pyridin-2-yl)piperidin-1-yl)methyl)-7-(difluoromethoxy)-1H-benzo[d]imidazole-6-carboxylic acid, RT=11.535 min, 2-((4-(6-((4-Chloro-2-fluorobenzyl)oxy)pyridin-2-yl)piperidin-1-yl)methyl)-7-hydroxy-1H-benzo[d]imidazole-6-carboxylic acid, RT=10.994 min) showed 4 was consumed completely. The reaction mixture was filtered. The filtrate was purified by prep-HPLC (column: Waters Xbridge BEH C₁₈ 100*30 mm*10 um; mobile phase: [H₂O (10 mM NH₄HCO₃)-ACN]; gradient: 20%-55% B over 8.0 min). The residue was repurified by prep-HPLC (column: Phenomenex Gemini-NX 80*40 mm*3 um; mobile phase: [H₂O (0.04% HCl)-ACN]; gradient: 20%-50% B over 20.0 min). 2-((4-(6-((4-Chloro-2-fluorobenzyl)oxy)pyridin-2-yl)piperidin-1-yl)methyl)-7-(difluoromethoxy)-1H-benzo[d]imidazole-6-carboxylic acid (5.03 mg, 8.97 μmol, 25% yield) was obtained as a pink solid.

LCMS: RT=2.262 min, MS cal.: 560.1, [M+H]⁺=561.2

HPLC: RT=11.485 min, purity: 90.68%

¹H NMR (400 MHz, MeOH-d4) δ=7.94-7.89 (m, 1H), 7.68 (t, J=7.6 Hz, 1H), 7.59 (d, J=8.4 Hz, 1H), 7.53-7.47 (m, 1H), 7.34 (t J=76 Hz, 1H), 7.27-7.18 (m, 2H), 6.93 (d, J=7.2 Hz, 1H), 6.76 (d, J=8.4 Hz, 1H), 5.43 (s, 2H), 4.72-4.66 (m, 2H), 3.87 (d, J=11.6 Hz, 2H), 3.47-3.38 (m, 2H), 3.11-2.98 (m, 1H), 2.23 (s, 4H)

2-((4-(6-((4-Chloro-2-fluorobenzyl)oxy)pyridin-2-yl)piperidin-1-yl)methyl)-7-hydroxy-1H-benzo[d]imidazole-6-carboxylic acid (1.74 mg, 3.41 μmol, 9% yield) was obtained as a pink solid.

LCMS: RT=2.650 min, MS cal.: 510.1, [M+H]⁺=511.2

HPLC: RT=10.988 min, purity: 96.12%

¹H NMR (400 MHz, MeOH-d4) δ=7.86 (d, J=8.8 Hz, 1H), 7.70 (t, J=7.6 Hz, 1H), 7.51 (t, J=8.4 Hz, 1H), 7.29-7.15 (m, 3H), 6.95 (d, J=7.2 Hz, 1H), 6.78 (d, J=8.4 Hz, 1H), 5.46-5.38 (m, 2H), 4.69 (s, 2H), 3.82 (d, J=11.6 Hz, 2H), 3.38 (d, J=3.6 Hz, 2H), 3.11-2.98 (m, 1H), 2.31-2.17 (m, 4H)

### Example A134—Preparation of Compound 211. (S)-2-((4-(6-((4-Chloro-2-fluorobenzyl)oxy)pyridin-2-yl)piperidin-1-yl)methyl)-1-methyl-4-(oxetan-2-ylmethoxy)-1H-benzo[d]imidazole-6-carboxylic acid (Compound 211) (3)

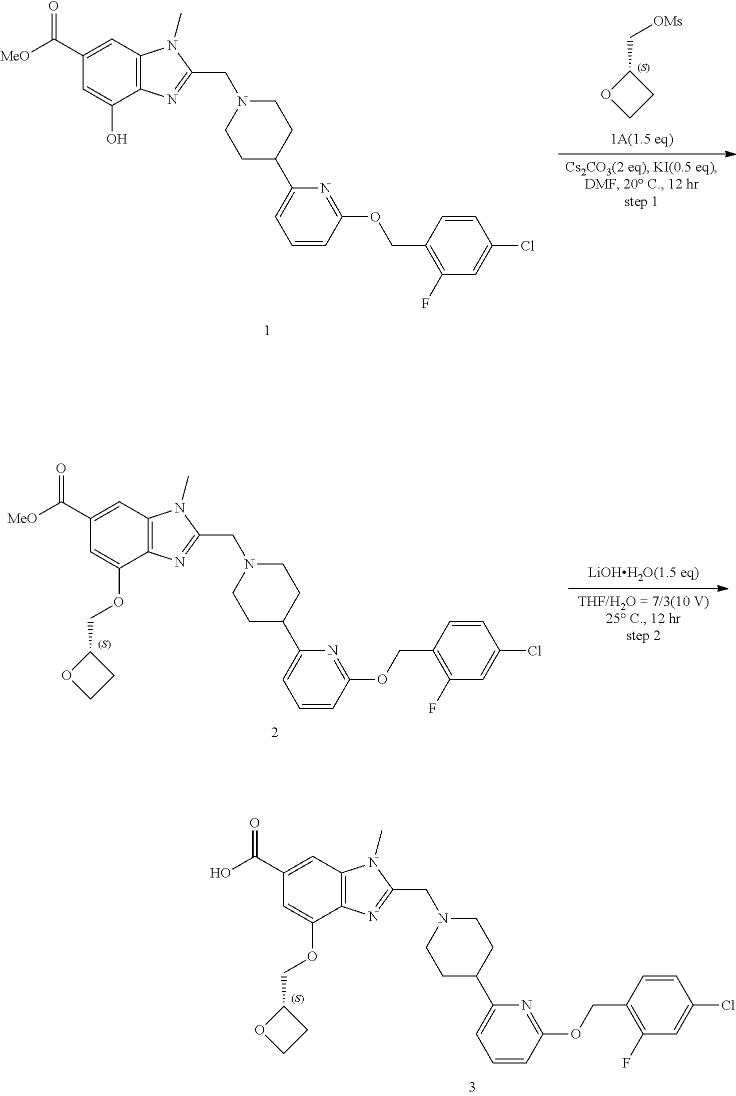

### General Procedure for Preparation of Intermediate 2:

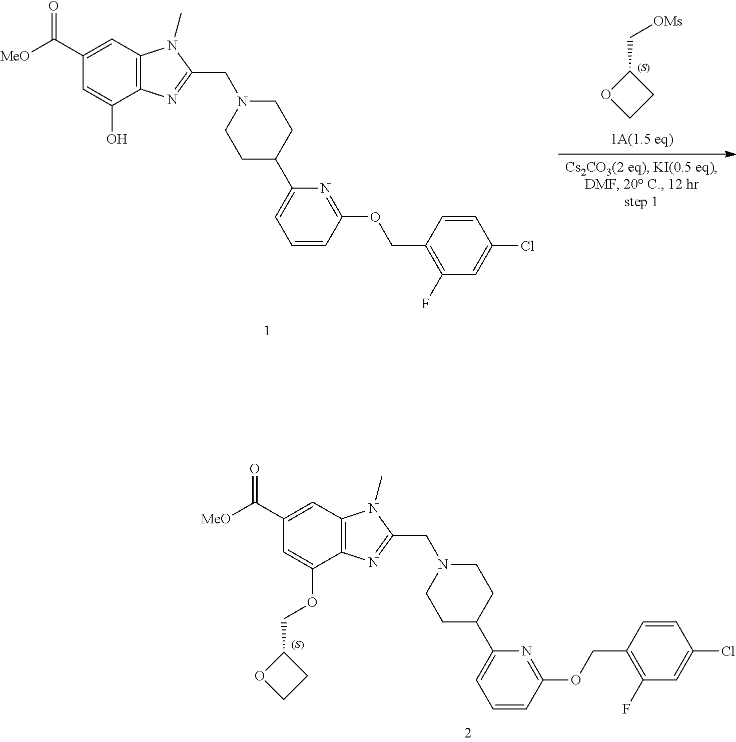

A mixture of 1 (60 mg, 111.32 μmol, 1 eq), 1A (27.75 mg, 166.98 μmol, 1.5 eq), Cs₂CO₃ (72.54 mg, 222.64 μmol, 2 eq), KI (9.24 mg, 55.66 μmol, 0.5 eq) in DMF (0.6 mL) was degassed and purged with N₂ for 3 times, and then the mixture was stirred at 20° C. for 12 hrs under N₂ atmosphere. 1A (27.75 mg, 166.98 μmol, 1.5 eq), Cs₂CO₃ (72.54 mg, 222.64 μmol, 2 eq), KI (9.24 mg, 55.66 μmol, 0.5 eq) were added to the mixture reaction and then the mixture was stirred at 20° C. for 12 hrs under N₂ atmosphere. LC-MS showed 1 was consumed completely and one main peak with desired mass was detected. The residue mixture was diluted with H₂O 10 mL) and extracted with EtOAc (10 mL*3). The combined organic layers were washed with brine 10 mL, dried over Na₂SO₄, filtered, and concentrated under reduced pressure to give a residue. The residue was purified by column chromatography (SiO₂, Petroleum ether/Ethyl acetate=100/1 to 0/1, TLC-Petroleum ether/Ethyl acetate=0/1, Product R_{f}=0.10). 2 (50 mg, 75.93 μmol, 51% yield) was obtained as a colorless oil.

LCMS: RT=0.750 min, MS cal.: 608.22, [M+H]⁺=609.3

¹H NMR (400 MHz, CHCl3-d) δ=7.79 (d, J=0.8 Hz, 1H), 7.53-7.42 (m, 3H), 7.14-7.08 (m, 2H), 6.73 (d, J=7.2 Hz, 1H), 6.61 (d, J=8.0 Hz, 1H), 5.41 (s, 2H), 5.35-5.25 (m, 1H), 4.77-4.64 (m, 2H), 4.57-4.46 (m, 2H), 3.98 (br s, 3H), 3.96 (s, 3H), 3.90 (br s, 2H), 2.96 (br d, J=4.0 Hz, 2H), 2.90-2.81 (m, 1H), 2.78-2.68 (m, 1H), 2.67-2.54 (m, 1H), 2.38-2.18 (m, 2H), 1.94-1.78 (m, 4H)

### General Procedure for Preparation of Compound 211 (3):

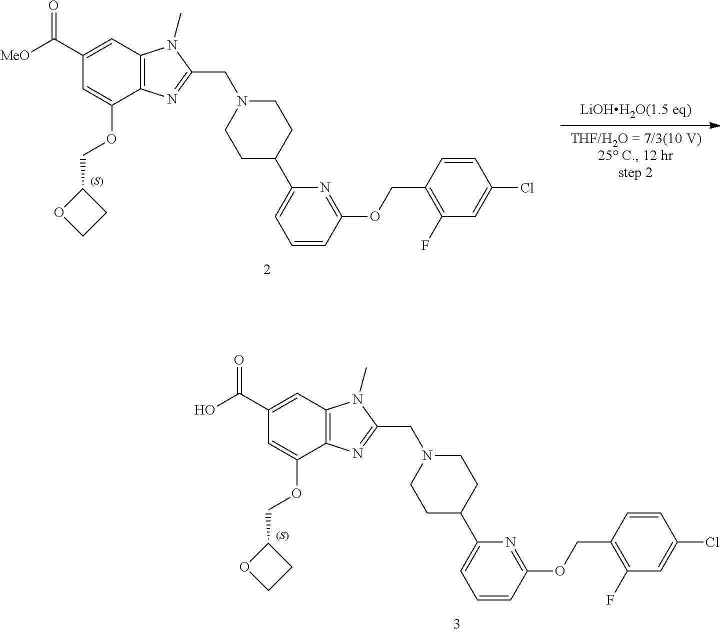

A mixture of 2 (40 mg, 65.67 μmol, 1 eq), LiOH·H₂O (4.13 mg, 98.51 μmol, 1.5 eq) in THF (0.28 mL) and H₂O (0.12 mL) was degassed and purged with N₂ for 3 times, and then the mixture was stirred at 25° C. for 12 hrs under N₂ atmosphere. LC-MS showed 2 was consumed completely and one main peak with desired mass was detected. The reaction mixture was diluted with ACN 0.5 mL. The liquid was purified by prep-HPLC (column: Waters Xbridge Prep OBD C₁₈ 150*40 mm*10 um; mobile phase: [H₂O (10 mM NH₄HCO₃)-ACN]; gradient: 25%-75% B over 8.0 min). (S)-2-((4-(6-((4-Chloro-2-fluorobenzyl)oxy)pyridin-2-yl)piperidin-1-yl)methyl)-1-methyl-4-(oxetan-2-ylmethoxy)-1H-benzo[d]imidazole-6-carboxylic acid (25.24 mg, 42.14 μmol, 51% yield, 99.35% purity) was obtained as a white solid.

LCMS: RT=0.696 min, MS cal.: 594.20, [M+H]⁺=595.2

¹H NMR (400 MHz, DMSO-d₆) δ=7.80 (s, 1H), 7.61 (t, J=7.6 Hz, 1H), 7.55 (t, J=8.0 Hz, 1H), 7.44 (dd, J=1.6, 10.0 Hz, 1H), 7.32 (s, 1H), 7.28 (dd, J=1.6, 8.2 Hz, 1H), 6.86 (d, J=7.2 Hz, 1H), 6.66 (d, J=8.0 Hz, 1H), 5.35 (s, 2H), 5.13-5.05 (m, 1H), 4.59-4.50 (m, 2H), 4.46-4.39 (m, 1H), 4.39-4.32 (m, 1H), 3.91 (s, 3H), 3.81 (s, 2H), 2.92 (br d, J=11.2 Hz, 2H), 2.80-2.68 (m, 1H), 2.65-2.54 (m, 2H), 2.19 (br t, J=10.8 Hz, 2H), 1.82-1.63 (m, 4H)

### Example A135—Preparation of Compound 212. 2-((4-(6-((4-Chloro-2-fluoro-3-methoxybenzyl)oxy)pyridin-2-yl)piperidin-1-yl)methyl)-4-(difluoromethoxy)-1-methyl-1H-benzo[d]imidazole-6-carboxylic acid (Compound 212) (3)

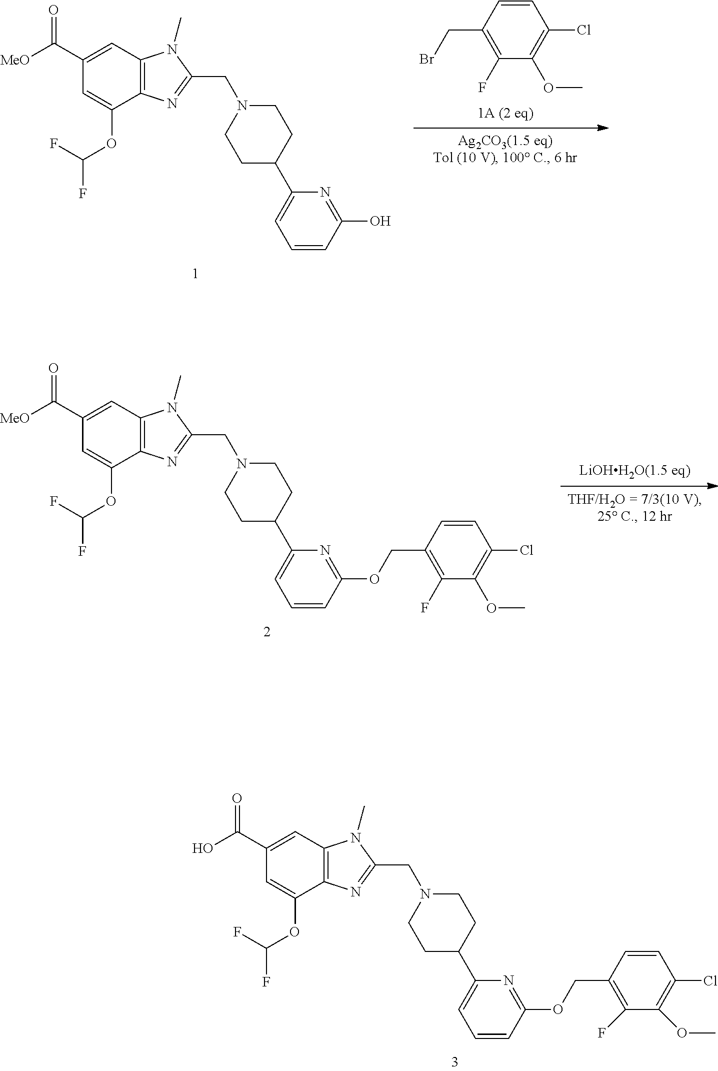

### General Procedure for Preparation of Intermediate 2:

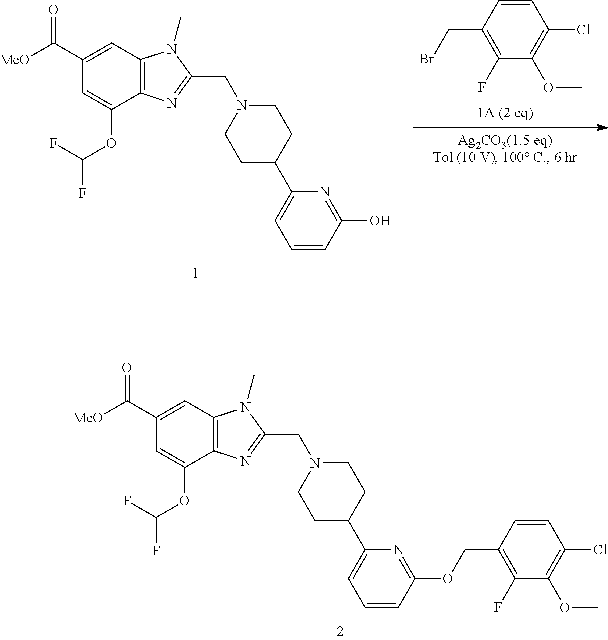

A mixture of 1 (0.15 g, 335.99 μmol, 1 eq), 1A (170.34 mg, 671.97 μmol, 2 eq) in Toluene (2 mL) was degassed and purged with N₂ for 3 times, then Ag₂CO₃ (138.97 mg, 503.98 μmol, 22.87 μL, 1.5 eq) was added to the mixture. The mixture was stirred at 100° C. for 6 hr under N₂ atmosphere. LCMS showed 1 was consumed completely and one main peak with desired m/z was detected. The reaction mixture was filtered and concentrated under reduced pressure to give a residue. The residue was purified by column chromatography (SiO₂, Petroleum ether/Ethyl acetate=20/1 to 3/1). 2 (0.095 g, 127.38 μmol, 37.91% yield, 83% purity) was obtained as a faint yellow solid.

LCMS: RT=1.745 min, MS cal.: 618.19, [M+H]⁺=619.1

### General Procedure for Preparation of Compound 212 (3):

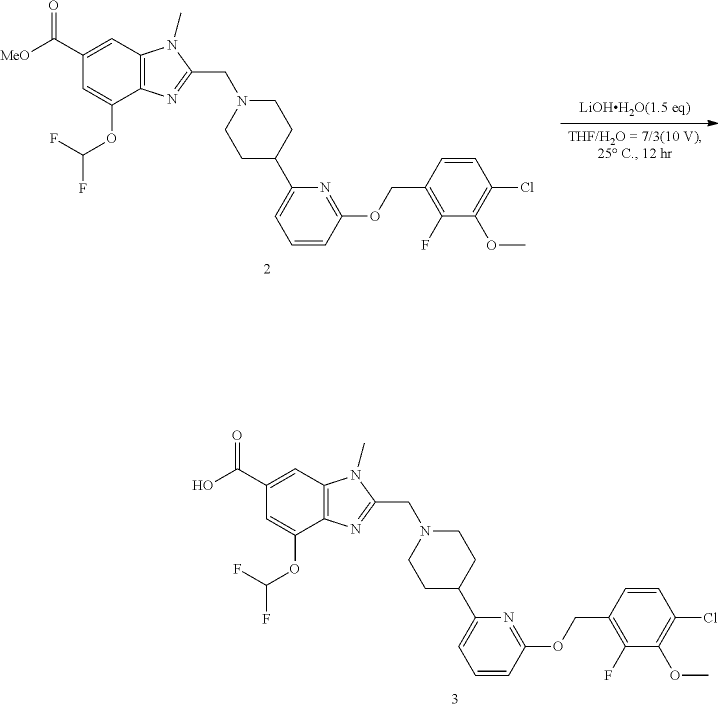

A mixture of 2 (70 mg, 113.08 μmol, 1 eq), LiOH·H₂O (7.12 mg, 169.62 μmol, 1.5 eq) in THF (0.7 mL) and H₂O (0.3 mL) was degassed and purged with N₂ for 3 times, and then the mixture was stirred at 25° C. for 12 hr under N₂ atmosphere. LC-MS (RT=1.235 min) showed 2 was consumed completely and one main peak with desired mass was detected. The reaction was purified directly. The residue was purified by prep-HPLC (column: Waters Xbridge Prep OBD C18 150*40 mm*10 um; mobile phase: [H₂O (10 mM NH₄HCO₃)-ACN]; gradient: 15%-65% B over 8.0 min). 2-((4-(6-((4-Chloro-2-fluoro-3-methoxybenzyl)oxy)pyridin-2-yl)piperidin-1-yl)methyl)-4-(difluoromethoxy)-1-methyl-1H-benzo[d]imidazole-6-carboxylic acid (29.38 mg, 47.29 μmol, 41.82% yield, 97.38% purity) was obtained as a white solid.

LCMS: RT=2.493 min, MS cal.: 604.17, [M+1]⁺=605.2

HPLC: RT=12.51 min, purity: 97.38%

¹H NMR (400 MHz, DMSO) δ=8.06 (d, J=1.2 Hz, 1H), 7.65-7.59 (m, 2H), 7.62 (t, J=74.4 Hz), 7.52 (s, 1H), 7.31-7.22 (m, 2H), 6.86 (d, J=7.2 Hz, 1H), 6.67 (d, J=8.4 Hz, 1H), 5.36 (s, 2H), 3.96 (s, 3H), 3.84 (s, 5H), 2.93 (br d, J=11.2 Hz, 2H), 2.62-2.54 (m, 1H), 2.27-2.15 (m, 2H), 1.82-1.62 (m, 4H)

### Example A136—Preparation of Compound 213. 2-((4-(6-((5-Cyanopyridin-2-yl)methoxy)pyridin-2-yl)piperidin-1-yl)methyl)-4-(difluoromethoxy)-1-methyl-1H-benzo[d]imidazole-6-carboxylic acid (Compound 213) (3)

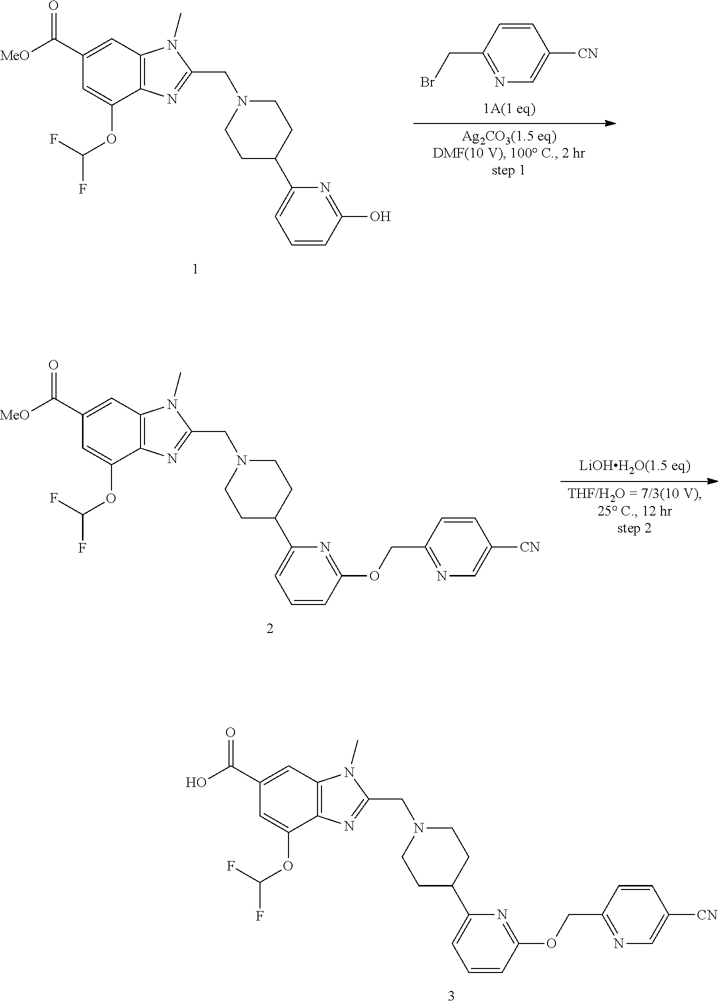

### General Procedure for Preparation of Intermediate 2:

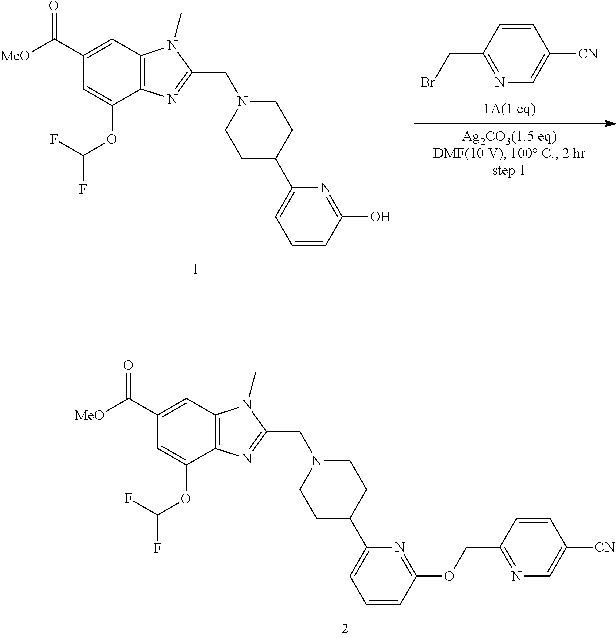

To a solution of 1 (0.1 g, 223.99 μmol, 1 eq) in DMF (1 mL) was added 1A (44.13 mg, 223.99 μmol, 1 eq), Ag₂CO₃ (92.65 mg, 335.99 μmol, 15.25 μL, 1.5 eq). The mixture was stirred at 100° C. for 2 hr. LCMS showed 1 was consumed and desired mass was detected. The reaction was quenched by addition of 3 mL of H₂O at 25° C. The reaction mixture was partitioned between EtOAc and water. The mixture was poured into separatory funnel and separated. The aqueous layer was extracted with EtOAc (3 mL*3). The combined organic layer was washed with brine 4 mL (2 mL*2), dried over Na₂SO₄, filtered and the filtrate was concentrated to give a crude product. The residue was purified by Prep-HPLC (column: 2_Phenomenex Gemini C18 75*40 mm*3 um; mobile phase: [H₂O (10 mM NH₄HCO₃)-ACN]; gradient: 40%-65% B over 8.0 min). 2 (80 mg, 142.21 μmol, 63% yield) was obtained as a yellow solid.

LCMS: RT=0.432 min, MS cal.: 562.2, 563.2, [M+H]⁺=563.2, 564.2

¹H NMR (400 MHz, DMSO-d6) δ=8.93 (d, J=1.2 Hz, 1H), 8.20 (dd, J=8.4, 2.0 Hz, 1H), 8.09 (d, J=1.2 Hz, 1H), 7.62 (t, J=7.8 Hz, 1H), 7.58 (t, J=74.4 Hz, 1H), 7.57-7.50 (m, 2H), 6.83 (d, J=7.4 Hz, 1H), 6.74 (d, J=8.0 Hz, 1H), 5.47 (s, 2H), 3.94 (s, 3H), 3.88 (s, 3H), 3.81 (s, 2H), 2.85 (br d, J=11.6 Hz, 2H), 2.55-2.52 (m, 1H), 2.16 (t, J=10.8 Hz, 2H), 1.71-1.63 (m, 2H), 1.60-1.48 (m, 2H)

### General Procedure for Preparation of Compound 213 (3):

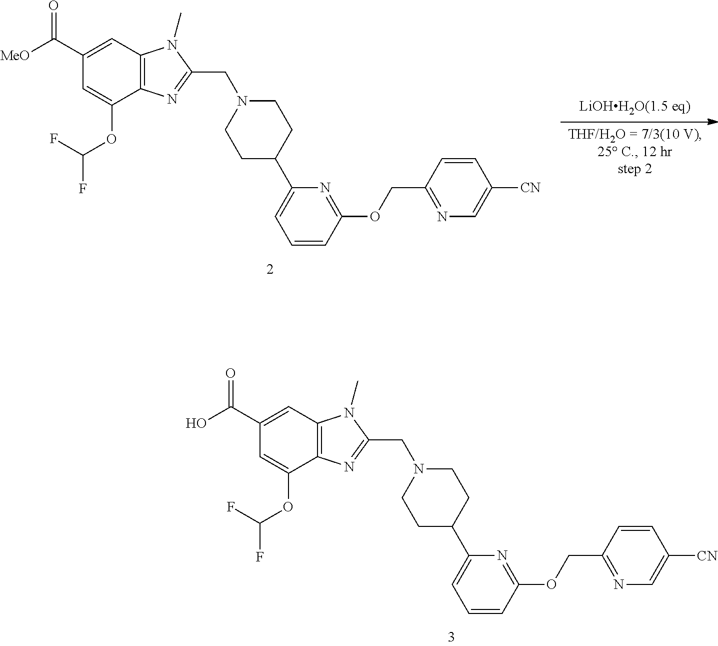

To a solution of 2 (75 mg, 133.32 μmol, 1 eq) in THF (0.52 mL) and H₂O (0.22 mL) was added LiOH·H₂O (8.39 mg, 199.98 μmol, 1.5 eq). The mixture was stirred at 25° C. for 12 hr. LCMS showed 2 was consumed and desired mass was detected. The reaction mixture was filtered and concentrated under reduced pressure to give a residue. The residue was purified by Prep-HPLC (column: Waters Xbridge Prep OBD C18 150*40 mm*10 um; mobile phase: [column: Waters Xbridge Prep OBD C18 150*40 mm*10 um; mobile phase: [H₂O (10 mM NH₄HCO₃)-ACN]; gradient: 15%-55% B over 8.0 min). 2-((4-(6-((4-Chloro-2-fluoro-3-methoxybenzyl)oxy)pyridin-2-yl)piperidin-1-yl)methyl)-4-(difluoromethoxy)-1-methyl-1H-benzo[d]imidazole-6-carboxylic acid (14.95 mg, 26.60 μmol, 19.95% yield, 97.6% purity) was obtained as a white solid.

LCMS: RT=2.439 min, MS cal.: 548.2, 549.2, [M+H]⁺=549.2, 550.2

HPLC: RT=9.351 purity: 97.6%

¹H NMR (400 MHz, DMSO-d6) δ=8.99 (d, J=1.6 Hz, 1H), 8.28 (dd, J=8.4, 2.0 Hz, 1H), 8.06 (d, J=1.0 Hz, 1H), 7.66 (t, J=7.8 Hz, 1H), 7.62 (t, J=74.4 Hz, 1H), 7.58 (d, J=8.0 Hz, 1H), 7.51 (s, 1H), 6.87 (d, J=7.6 Hz, 1H), 6.77 (d, J=8.0 Hz, 1H), 5.49 (s, 2H), 3.95 (s, 3H), 3.82 (s, 2H), 2.87 (d, J=11.2 Hz, 2H), 2.55 (s, 1H), 2.16 (t, J=10.8 Hz, 2H), 1.73-1.62 (m, 2H), 1.60-1.48 (m, 2H)

### Example A137—Preparation of Compound 214. 2-((4-(6-((4-Chloro-2-fluorobenzyl)oxy)pyridin-2-yl)piperidin-1-yl)methyl)-4-((1-cyanocyclopropyl)methoxy)-1-methyl-1H-benzo[d]imidazole-6-carboxylic acid (Compound 214) (3)

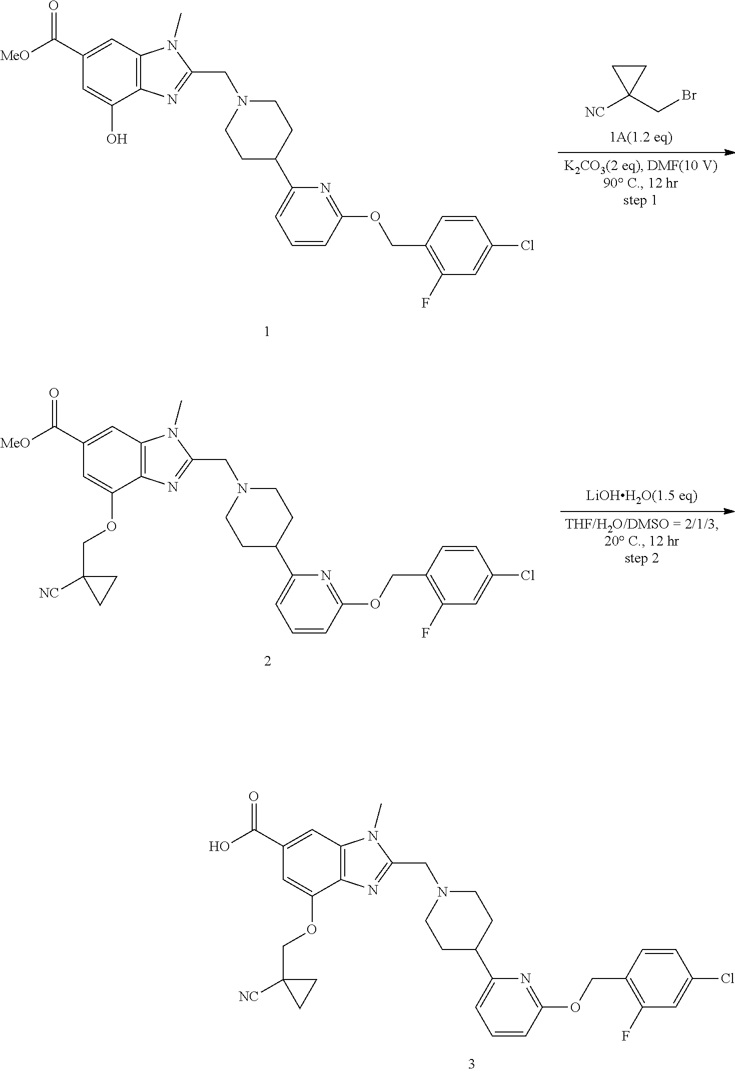

### General Procedure for Preparation of Intermediate 2:

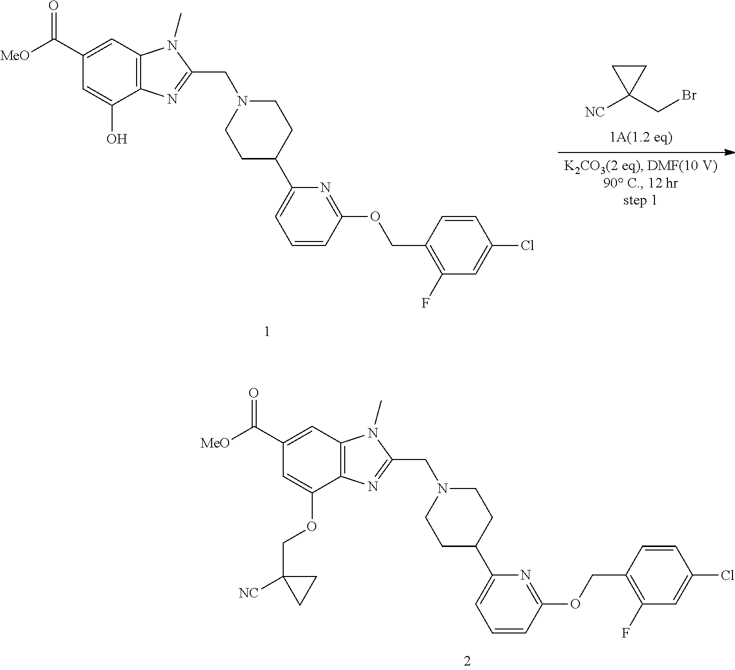

A mixture of 1 (0.06 g, 111.32 μmol, 1 eq), 1A (21.37 mg, 133.58 μmol, 1.2 eq), K₂CO₃ (30.77 mg, 222.64 μmol, 2 eq) in DMF (1 mL) was degassed and purged with N₂ for 3 times, and then the mixture was stirred at 90° C. for 12 hr under N₂ atmosphere. TLC (PE/EtOAc=0/1, 1 Rf=0.3) showed the 1 was consumed completely. The reaction mixture was added H₂O 5 mL and extracted with EtOAc 5 mL*3. The combined organic layers were washed with brine 5 mL*3, dried over Na₂SO₄, filtered and concentrated under reduced pressure to give a residue. The residue was purified by prep-TLC (SiO₂, PE/EtOAc=0/1). 2 (0.05 g, 64.71 μmol, 58.14% yield, 80% purity) was obtained as a faint yellow oil.

¹H NMR (400 MHz, CHCl3-d) δ=7.82 (s, 1H), 7.51 (br t, J=8.0 Hz, 1H), 7.47-7.41 (m, 2H), 7.16-7.07 (m, 2H), 6.77-6.72 (m, 1H), 6.61 (br d, J=7.6 Hz, 1H), 5.43-5.38 (m, 2H), 4.42 (br s, 2H), 4.02-3.95 (m, 6H), 3.94-3.86 (m, 2H), 3.01-2.92 (m, 2H), 2.67-2.55 (m, 1H), 2.37-2.24 (m, 1H), 2.36-2.24 (m, 1H), 2.09-2.04 (m, 1H), 1.93-1.77 (m, 4H), 1.67-1.51 (m, 5H), 1.43 (br s, 2H), 1.23-1.19 (m, 1H), 1.31-1.19 (m, 4H).

### General Procedure for Preparation of Compound 214 (3):

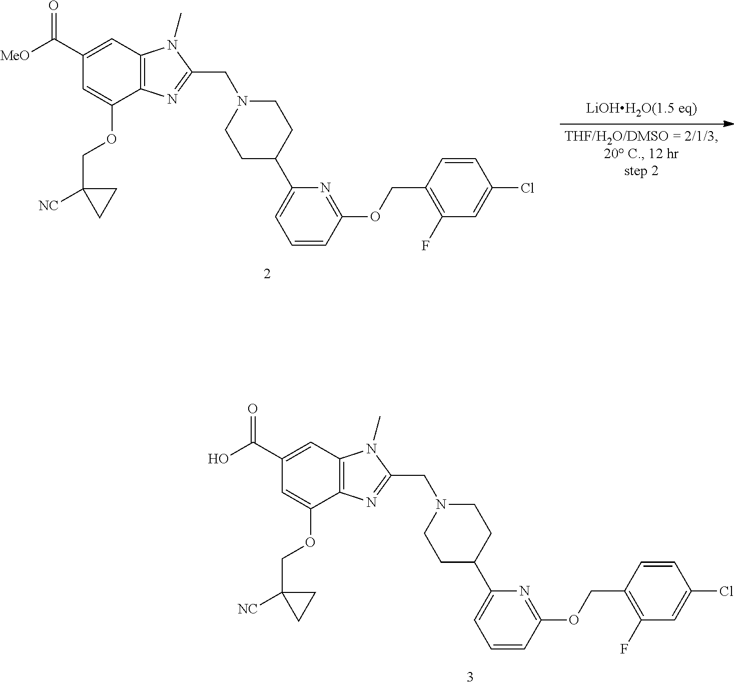

A mixture of 2 (50 mg, 80.89 μmol, 1 eq), in DMSO (0.9 mL) and THF (0.6 mL) LiOH·H₂O (5.09 mg, 121.34 μmol, 1.5 eq) in H₂O (0.3 mL) was degassed and purged with N₂ for 3 times, and then the mixture was stirred at 20° C. for 12 hr under N₂ atmosphere. LCMS (ET64759-607-P1Q, product: RT=1.197 min) showed the 2 was consumed completely. The reaction mixture was filtered. The filtrate was purified by prep-HPLC (column: Waters Xbridge Prep OBD C18 150*40 mm*10 um; mobile phase: [H₂O (10 mM NH₄HCO₃)-ACN]; gradient: 30%-60% B over 8.0 min). 2-((4-(6-((4-Chloro-2-fluorobenzyl)oxy)pyridin-2-yl)piperidin-1-yl)methyl)-4-((1-cyanocyclopropyl)methoxy)-1-methyl-1H-benzo[d]imidazole-6-carboxylic acid (24.14 mg, 39.96 μmol, 49.40% yield) was obtained as a white solid.

LCMS: RT=2.769 min, MS cal.: 603.2, [M+H]⁺=604.2

HPLC: RT=12.197 min

¹H NMR (400 MHz, DMSO-d6) δ=7.86-7.75 (m, 1H), 7.63 (t, J=7.8 Hz, 1H), 7.56 (t, J=8.4 Hz, 1H), 7.45 (dd, J=2.0, 10.0 Hz, 1H), 7.29 (dd, J=1.6, 8.4 Hz, 1H), 7.23-7.12 (m, 1H), 6.87 (d, J=7.2 Hz, 1H), 6.67 (d, J=8.4 Hz, 1H), 5.36 (s, 2H), 4.29 (br s, 2H), 3.91 (br s, 3H), 3.83 (br s, 2H), 2.93 (br d, J=10.8 Hz, 2H), 2.64-2.56 (m, 1H), 2.21 (br t, J=11.2 Hz, 2H), 1.87-1.63 (m, 4H), 1.46-1.35 (m, 2H), 1.29-1.18 (m, 2H)

### Example A138—Preparation of Compound 215. 2-((4-(6-((4-Chloro-2-fluorobenzyl)oxy)pyridin-2-yl)piperidin-1-yl)methyl)-4-cyclopropoxy-1-methyl-1H-benzo[d]imidazole-6-carboxylic acid (Compound 215) (7)

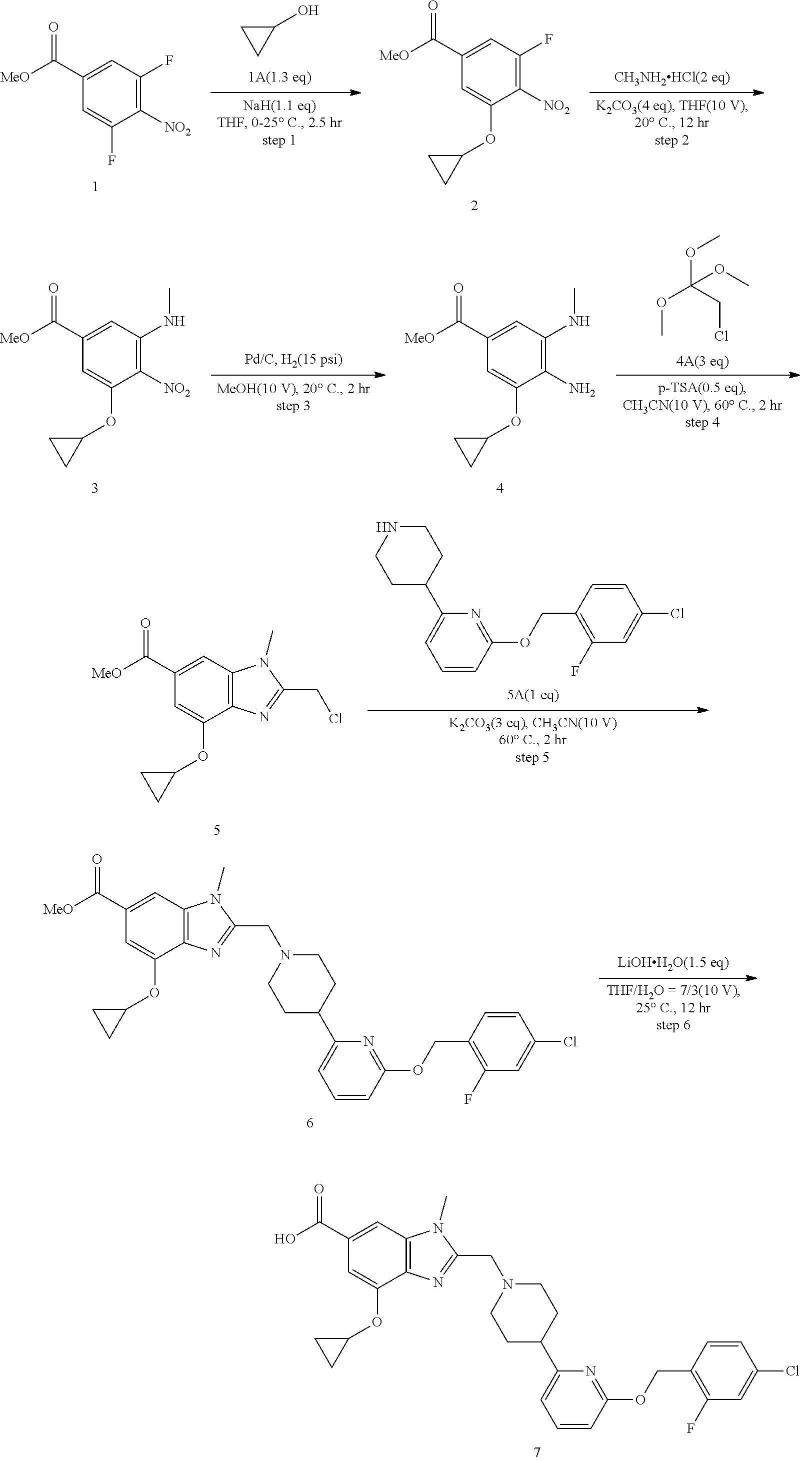

### General Procedure for Preparation of Intermediate 2:

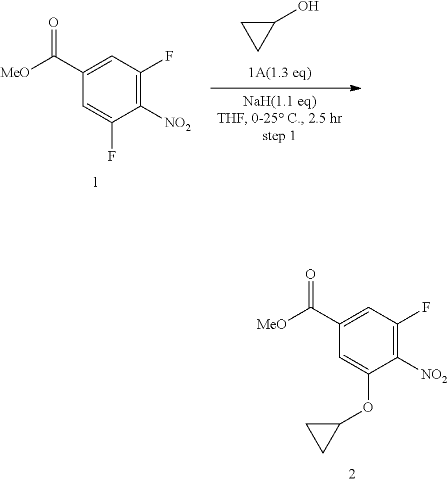

To a solution of 1A (521.60 mg, 8.98 mmol, 1.3 eq) in THF (7.5 mL) was added NaH (303.94 mg, 7.60 mmol, 60% purity, 1.1 eq) at 0° C. The mixture was stirred at at 0° C. for 0.5 hr. 1 (1.5 g, 6.91 mmol, 1 eq) was added to the reaction mixture at 0° C. The mixture was stirred at 25° C. for 2 hr. TLC (Petroleum ether/Ethyl acetate=5/1 1 R_{f}=0.60, product R_{f}=0.53) indicated 1 was consumed completely and one new spot formed. The reaction mixture was added to H₂O 20 mL at 20° C. and extracted by ethyl acetate (15 mL*3). Then organic phase was combined and washed by brine 10 mL*2, dried with Na₂SO₄, filtered. The organic phase was concentrated under reduced pressure at 35° C. to give a residue. The residue was purified by column chromatography (SiO₂, Petroleum ether/Ethyl acetate=1/0 to 5/1). 2 (780 mg, 3.06 mmol, 44.24% yield) was obtained as a white solid.

¹H NMR (400 MHz, CHCl3-d) δ=7.85 (s, 1H), 7.52 (dd, J=1.2, 9.2 Hz, 1H), 3.98 (s, 3H), 3.97-3.94 (m, 1H), 0.93-0.85 (m, 4H)

### General Procedure for Preparation of Intermediate 3:

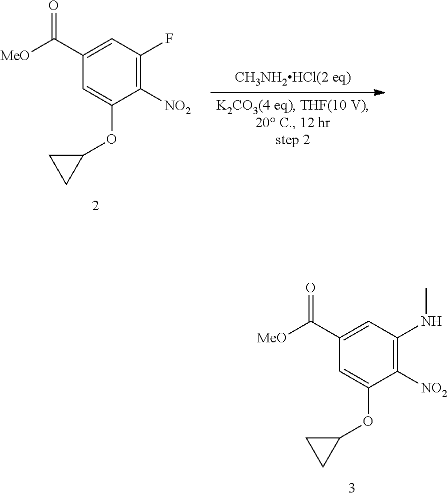 [SMILES: COC(=O)c1cc(F)c([N+](=O)[O-])c(OC2CC2)c1 ; "CH3NH2·HCl(2 eq)" ; "K2CO3(4 eq), THF(10 V),20° C., 12 hrstep 2" ; CNc1cc(C(=O)OC)cc(OC2CC2)c1[N+](=O)[O-]]

A mixture of 2 (700 mg, 2.74 mmol, 1 eq), CH₃NH₂·HCl (370.40 mg, 5.49 mmol, 2 eq), K₂CO₃ (1.52 g, 10.97 mmol, 4 eq) in THF (7 mL) was degassed and purged with N₂ for 3 times, and then the mixture was stirred at 20° C. for 12 hr under N₂ atmosphere. HPLC (product: RT=2.295 min) showed the 2 was consumed completely. The reaction mixture was quenched by addition H₂O 5 mL and extracted with ethyl acetate 15 mL (5 mL*3). The combined organic layers were washed with brine 10 mL (5 mL*2), dried over Na₂SO₄, filtered, and concentrated under reduced pressure to give a residue. 3 (680 mg, 2.55 mmol, 93.11% yield) was obtained as a red solid.

¹H NMR (400 MHz, CHCl3-d) δ=7.28 (d, J=1.6 Hz, 1H), 7.08 (d, J=1.2 Hz, 1H), 6.08-5.63 (m, 1H), 3.95 (s, 3H), 2.98-2.94 (m, 3H), 0.86 (s, 2H), 0.85 (s, 2H)

### General Procedure for Preparation of Intermediate 4:

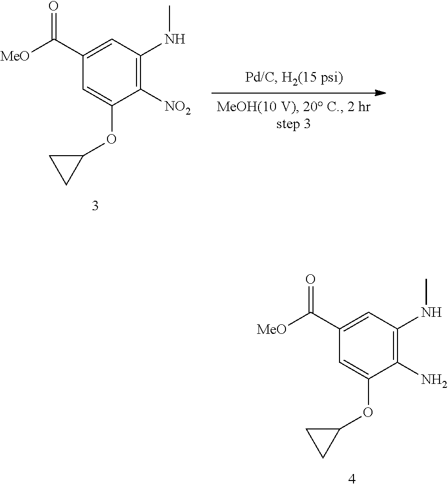 [SMILES: CNc1cc(C(=O)OC)cc(OC2CC2)c1[N+](=O)[O-] ; "MeOH(10 V), 20° C., 2 hrstep 3" ; "Pd/C, H2(15 psi)" ; CNc1cc(C(=O)OC)cc(OC2CC2)c1N]

MeOH (3 mL) and was charged to the round bottom flask, then 3 (300 mg, 1.13 mmol, 1 eq) was added to the mixture at 20° C. Pd/C (60.00 mg) was added to the reaction mixture at 20° C., under Ar atmosphere. The mixture was stirred at 20° C. for 2 hr under H₂ (15 Psi) atmosphere. LCMS (product: RT=0.339 min) showed the 3 was consumed completely. The suspension was filtered through a pad of celite, and filter cake was washed with MeOH (20 mL*6). The combined filtrate was concentrated to give a product. 4 (300 mg, crude) was obtained as a brown oil.

### General Procedure for Preparation of Intermediate 5:

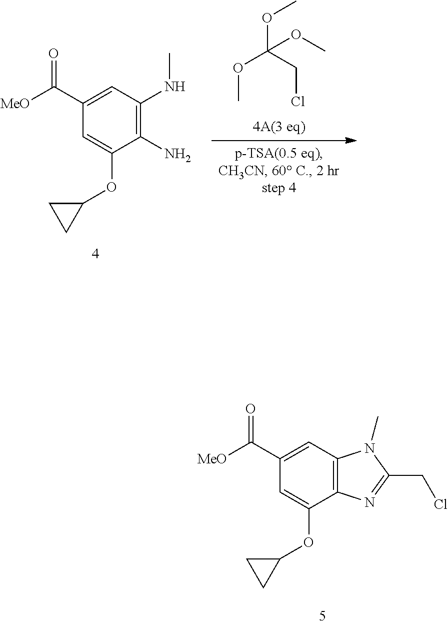

A mixture of 4 (250 mg, 1.06 mmol, 1 eq), 4A (490.73 mg, 3.17 mmol, 427.84 μL, 3 eq), p-TSA (91.11 mg, 529.06 μmol, 0.5 eq) in CH₃CN (2.5 mL) was degassed and purged with N₂ for 3 times, and then the mixture was stirred at 60° C. for 2 hr under N₂ atmosphere. LCMS (product: RT=0.433 min) showed the 4 was consumed completely. The reaction mixture was quenched by addition H₂O 10 mL at 20° C. and extracted with ethyl acetate 15 mL (5 mL*3). The combined organic layers were washed with brine 10 mL (5 mL*2), dried over Na₂SO₄, filtered, and concentrated under reduced pressure to give a residue. 5 (230 mg, crude) was obtained as a yellow oil.

### General Procedure for Preparation of Intermediate 6:

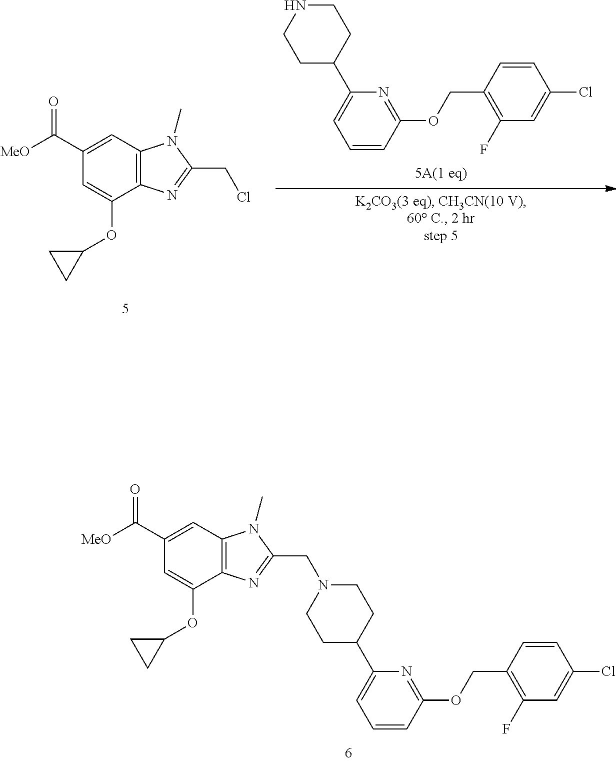 [SMILES: COC(=O)c1cc(OC2CC2)c2nc(CCl)n(C)c2c1 ; Fc1cc(Cl)ccc1COc1cccc(C2CCNCC2)n1 ; "5A(1 eq)" ; "K2CO3(3 eq), CH3CN(10 V),60° C., 2 hrstep 5" ; COC(=O)c1cc(OC2CC2)c2nc(CN3CCC(c4cccc(OCc5ccc(Cl)cc5F)n4)CC3)n(C)c2c1]

A mixture of methyl 5 (150 mg, 508.94 μmol, 1 eq), 5A (163.26 mg, 508.94 μmol, 1 eq), K₂CO₃ (211.01 mg, 1.53 mmol, 3 eq) in CH₃CN (1.5 mL) was degassed and purged with N₂ for 3 times, and then the mixture was stirred at 60° C. for 2 hr under N₂ atmosphere. LCMS (product: RT=0.508 min) showed the 5 was consumed completely. The reaction mixture was quenched by addition H₂O 5 mL at 20° C. and extracted with ethyl acetate 15 mL (5 mL*3). The combined organic layers were washed with brine 10 mL (5 mL*2), dried over Na₂SO₄, filtered, and concentrated under reduced pressure to give a residue. The residue was purified by column chromatography (SiO₂, Petroleum ether/Ethyl acetate=5/1 to 0/1). 6 (130 mg, 224.50 μmol, 44.11% yield) was obtained as a yellow solid.

¹H NMR (400 MHz, DMSO-d6) δ=7.85 (s, 1H), 7.62 (t, J=7.6 Hz, 1H), 7.59-7.52 (m, 2H), 7.45 (dd, J=2.0, 10.0 Hz, 1H), 7.28 (dd, J=1.6, 8.0 Hz, 1H), 6.86 (d, J=7.2 Hz, 1H), 6.67 (d, J=8.0 Hz, 1H), 5.35 (s, 2H), 4.08 (tt, J=2.8, 6.0 Hz, 1H), 3.92 (s, 3H), 3.88 (s, 3H), 3.86-3.73 (m, 2H), 2.92 (br s, 2H), 2.59 (br d, J=2.8 Hz, 1H), 2.20 (br s, 2H), 1.87-1.61 (m, 4H), 0.89-0.82 (m, 2H), 0.80-0.72 (m, 2H)

### General Procedure for Preparation of Compound 215 (7):

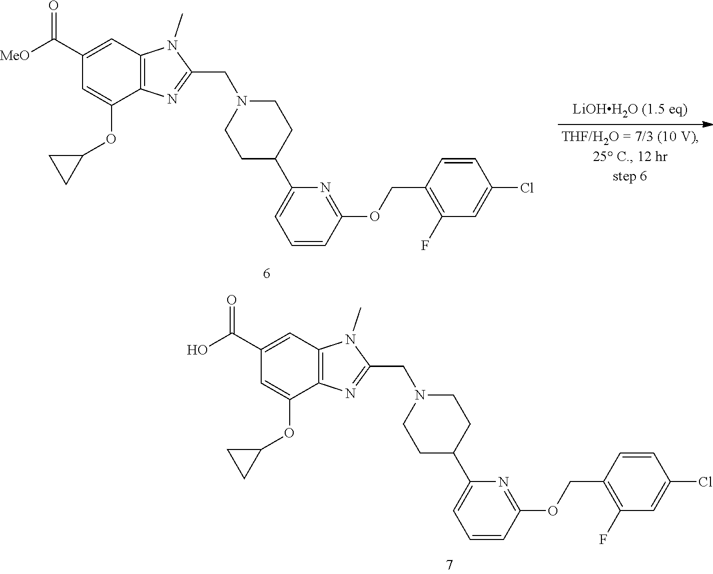 [SMILES: COC(=O)c1cc(OC2CC2)c2nc(CN3CCC(c4cccc(OCc5ccc(Cl)cc5F)n4)CC3)n(C)c2c1 ; Cn1c(CN2CCC(c3cccc(OCc4ccc(Cl)cc4F)n3)CC2)nc2c(OC3CC3)cc(C(=O)O)cc21]

To a solution of 6 (100 mg, 172.69 μmol, 1 eq) in THF (0.7 mL) was added LiOH·H₂O (10.87 mg, 259.04 μmol, 1.5 eq) in H₂O (0.3 mL). The mixture was stirred at 25° C. for 12 hr under N₂ atmosphere. LCMS (product: RT=1.184 min; start material: RT=1.728 min) showed 50% of 6 remained and 34% of desired Compound was detected. The reaction mixture was filtered, and the filtrate was purified by prep-HPLC (column: Waters Xbridge BEH C18 100*30 mm*10 um; mobile phase: [H₂O (10 mM NH₄HCO₃)—CH₃CN]; gradient: 35%-55% B over 8.0 min). 2-((4-(6-((4-Chloro-2-fluorobenzyl)oxy)pyridin-2-yl)piperidin-1-yl)methyl)-4-cyclopropoxy-1-methyl-1H-benzo[d]imidazole-6-carboxylic acid (29.55 mg, 51.49 μmol, 29.82% yield, 98.46% purity) was obtained as a white solid.

LCMS: RT=2.745 min, MS cal.: 564.2, [M+H]⁺=565.2

HPLC: RT=11.965 min

¹H NMR (400 MHz, MeOH-d4) δ=7.87 (s, 1H), 7.77 (s, 1H), 7.56 (t, J=7.6 Hz, 1H), 7.48 (t, J=8.4 Hz, 1H), 7.22-7.14 (m, 2H), 6.81 (d, J=7.6 Hz, 1H), 6.62 (d, J=8.4 Hz, 1H), 5.39 (s, 2H), 4.02 (tt, J=2.8, 5.6 Hz, 1H), 3.98 (s, 3H), 3.91 (s, 2H), 3.04 (br d, J=11.2 Hz, 2H), 2.69-2.59 (m, 1H), 2.40-2.29 (m, 2H), 1.91-1.81 (m, 4H), 0.93-0.87 (m, 2H), 0.87-0.82 (m, 2H)

### Example A139—Preparation of Compound 216. 2-((4-(3-((4-Chloro-2-fluorobenzyl)oxy)-4-fluorophenyl)piperidin-1-yl)methyl)-4-(difluoromethoxy)-1-methyl-1H-benzo[d]imidazole-6-carboxylic acid (Compound 216) (5)

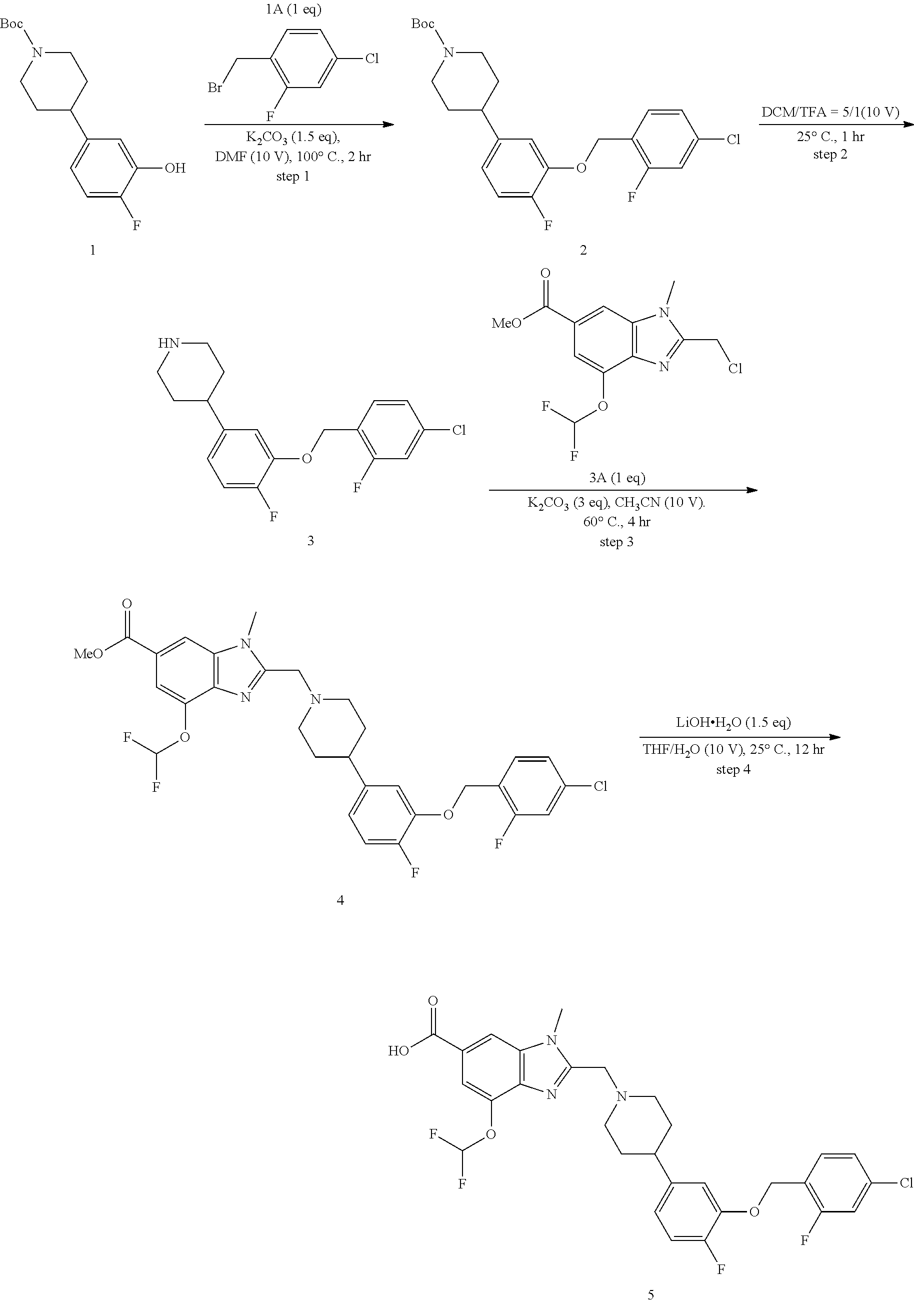

### General Procedure for Preparation of Intermediate 2:

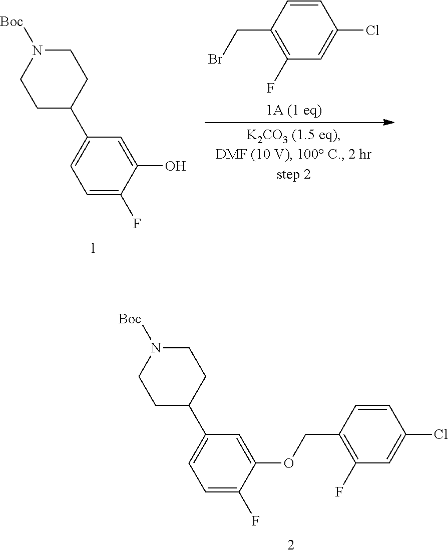 [SMILES: CC(C)(C)OC(=O)N1CCC(c2ccc(F)c(O)c2)CC1 ; Fc1cc(Cl)ccc1CBr ; CC(C)(C)OC(=O)N1CCC(c2ccc(F)c(OCc3ccc(Cl)cc3F)c2)CC1]

To a mixture of 1 (330 mg, 1.12 mmol, 1 eq) and 1A (249.69 mg, 1.12 mmol, 1 eq) in DMF (5 mL) was added K₂CO₃ (231.63 mg, 1.68 mmol, 1.5 eq). The mixture was stirred at 25° C. for 12 hours. The color changes to yellow. TLC (SiO₂, PE:EA=2:1, R_{f}=0.50) showed the reaction was completed. The reaction mixture was quenched by addition water 10 mL at 25° C., and then extracted with EA 15 mL (5 mL*3). The combined organic layers were washed with brine 50 mL (10 mL*5), dried over Na₂SO₄, filtered and concentrated under reduced pressure to give a residue. The residue was purified by prep-TLC (SiO₂, PE:EA=2:1, R_{f}=0.45). 2 (450 mg, 1.01 mmol, 90.76% yield) was obtained as yellow oil.

HPLC: RT=3.529 min, purity: 98.68%

¹H NMR (400 MHz, MeOH-d4) δ=7.52 (t, J=7.6 Hz, 1H), 7.30-7.20 (m, 2H), 7.07-6.96 (m, 2H), 6.86-6.78 (m, 1H), 5.16 (s, 2H), 4.19 (br d, J=13.2 Hz, 2H), 2.96-2.77 (m, 2H), 2.75-2.62 (m, 1H), 1.78 (br d, J=13.2 Hz, 2H), 1.61-1.50 (m, 2H), 1.48 (s, 9H)

### General Procedure for Preparation of Intermediate 3:

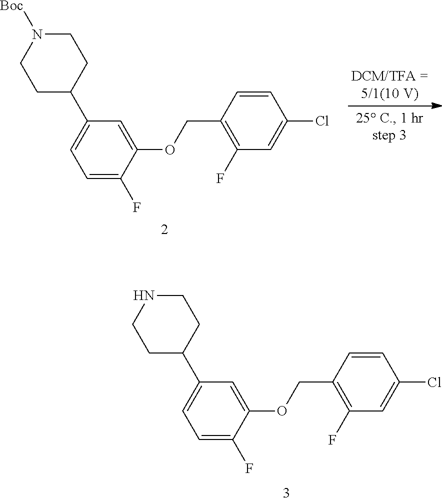 [SMILES: CC(C)(C)OC(=O)N1CCC(c2ccc(F)c(OCc3ccc(Cl)cc3F)c2)CC1 ; Fc1cc(Cl)ccc1COc1cc(C2CCNCC2)ccc1F]

A mixture of 2 (450 mg, 1.03 mmol, 1 eq) in DCM (4.5 mL) and TFA (0.9 mL) was stirred at 25° C. for 1 hour. The color changes to yellow. LCMS showed the reaction was completed. The reaction mixture was quenched by addition Sat. NaHCO₃10 mL at 25° C., and then diluted with EA 5 mL and extracted with EA 30 mL (10 mL*3). The combined organic layers were washed with brine (30 mL), dried over Na₂SO₄, filtered and concentrated under reduced pressure to give a residue. The mixture was used for the next step directly without purification. 3 (330 mg, 957.50 μmol, 93.18% yield) was obtained as yellow oil.

LCMS: RT=0.500 min, MS cal.: 337.1, [M+H]⁺=338.2

HPLC: RT=2.151 min, purity: 98.01%

¹H NMR (400 MHz, MeOH-d4) δ=7.53 (t, J=8.0 Hz, 1H), 7.28-7.21 (m, 2H), 7.07-6.99 (m, 2H), 6.84 (ddd, J=2.0, 4.4, 8.4 Hz, 1H), 5.17 (s, 2H), 3.18 (br d, J=12.4 Hz, 2H), 2.77 (dt, J=2.4, 12.4 Hz, 2H), 2.67 (tt, J=3.2, 12.0 Hz, 1H), 1.82 (br d, J=13.2 Hz, 2H), 1.71-1.57 (m, 2H)

### General Procedure for Preparation of Intermediate 4:

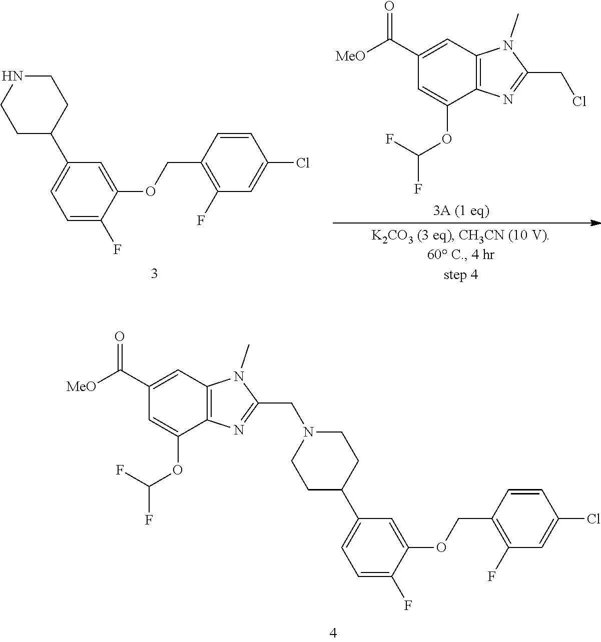 [SMILES: Fc1cc(Cl)ccc1COc1cc(C2CCNCC2)ccc1F ; COC(=O)c1cc(OC(F)F)c2nc(CCl)n(C)c2c1 ; COC(=O)c1cc(OC(F)F)c2nc(CN3CCC(c4ccc(F)c(OCc5ccc(Cl)cc5F)c4)CC3)n(C)c2c1]

A mixture of 3 (110 mg, 325.65 μmol, 1.2 eq), 3A (82.68 mg, 271.37 μmol, 1 eq) and K₂CO₃ (112.52 mg, 814.11 μmol, 3 eq) in ACN (1.5 mL) was stirred at 60° C. for 1 hour. The color changes to brown. TLC (SiO₂, PE:EA=1:1, R_{f}=0.50) showed the reaction was completed. The mixture was filtered and concentrated in vacuum. The residue was purified prep-TLC (SiO₂, PE:EA=1:1, R_{f}=0.45). 4 (150 mg, 242.42 μmol, 89.33% yield was obtained as yellow oil.

HPLC: RT=3.392 min, purity: 97.94%)

¹H NMR (400 MHz, MeOH-d4) δ=8.13 (d, J=1.2 Hz, 1H), 7.68 (s, 1H), 7.51 (t, J=8.4 Hz, 1H), 7.26 (t, J=74.0 Hz, 1H), 7.27-7.21 (m, 2H), 7.04-6.97 (m, 2H), 6.83 (ddd, J=2.0, 4.4, 8.4 Hz, 1H), 5.16 (s, 2H), 4.03 (s, 3H), 3.96 (s, 3H), 3.91 (s, 2H), 3.05-2.98 (m, 2H), 2.60-2.49 (m, 1H), 2.31 (dt, J=1.2, 10.8 Hz, 2H), 1.82-1.69 (m, 4H)

### General Procedure for Preparation of Compound 216 (5):

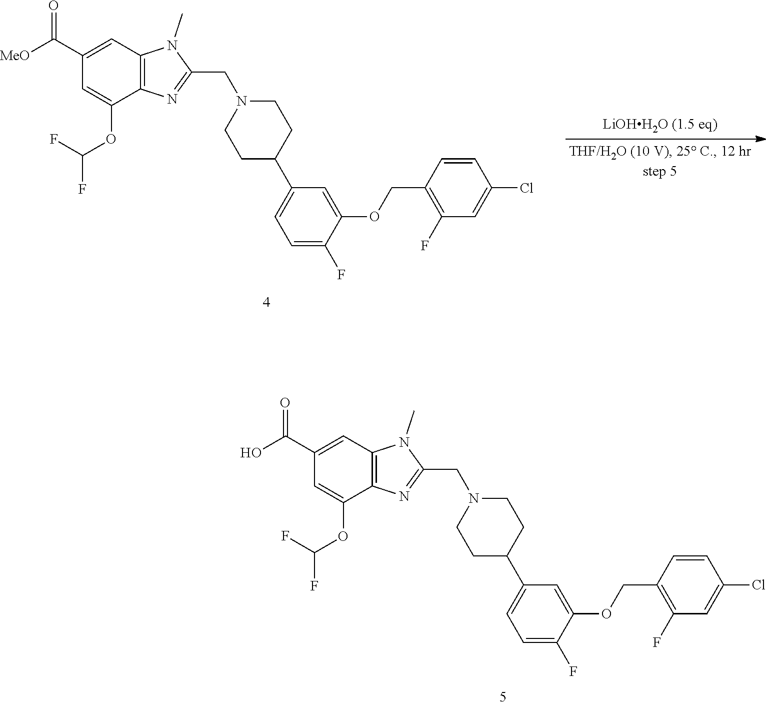 [SMILES: COC(=O)c1cc(OC(F)F)c2nc(CN3CCC(c4ccc(F)c(OCc5ccc(Cl)cc5F)c4)CC3)n(C)c2c1 ; Cn1c(CN2CCC(c3ccc(F)c(OCc4ccc(Cl)cc4F)c3)CC2)nc2c(OC(F)F)cc(C(=O)O)cc21]

To a mixture of 4 (150 mg, 247.52 μmol, 1 eq) and LiOH·H₂O (15.58 mg, 371.28 μmol, 1.5 eq) in THF (2.1 mL) and H₂O (0.9 mL) was stirred at 25° C. for 36 hours. The color changes to pink. LCMS showed 90% of desired product was detected. showed the reaction was completed.

The mixture was concentrated in vacuum. The residue was purified by prep-HPLC (column: Waters Xbridge Prep OBD C18 150*40 mm*10 um; mobile phase: [H₂O (10 mM NH₄HCO₃)-ACN]; gradient: 20%-70% B over 8.0 min). 2-((4-(3-((4-Chloro-2-fluorobenzyl)oxy)-4-fluorophenyl)piperidin-1-yl)methyl)-4-(difluoromethoxy)-1-methyl-1H-benzo[d]imidazole-6-carboxylic acid (103.58 mg, 171.84 μmol, 69.42% yield) was obtained as a white solid.

LCMS: RT=2.825 min, MS cal.: 596.2, [M+H]⁺=597.2

HPLC: RT=12.771 min, purity: 98.21%

¹H NMR (400 MHz, MeOH-d4) δ=8.10 (s, 1H), 7.68 (s, 1H), 7.51 (t, J=8.0 Hz, 1H), 7.24 (t, J=72.4 Hz, 1H), 7.26-7.21 (m, 2H), 7.04-6.98 (m, 2H), 6.83 (ddd, J=2.0, 4.4, 8.4 Hz, 1H), 5.16 (s, 2H), 4.01 (s, 3H), 3.96 (s, 2H), 3.07 (br d, J=11.2 Hz, 2H), 2.56 (tt, J=3.6, 12.0 Hz, 1H), 2.38 (br t, J=10.8 Hz, 2H), 1.86-1.68 (m, 4H)

### Example A140—Preparation of Compound 217. 2-((4-(3-((4-Chloro-2-fluorobenzyl)oxy)-1H-pyrazol-1-yl)piperidin-1-yl)methyl)-4-(difluoromethoxy)-1-methyl-1H-benzo[d]imidazole-6-carboxylic acid (Compound 217) (7)

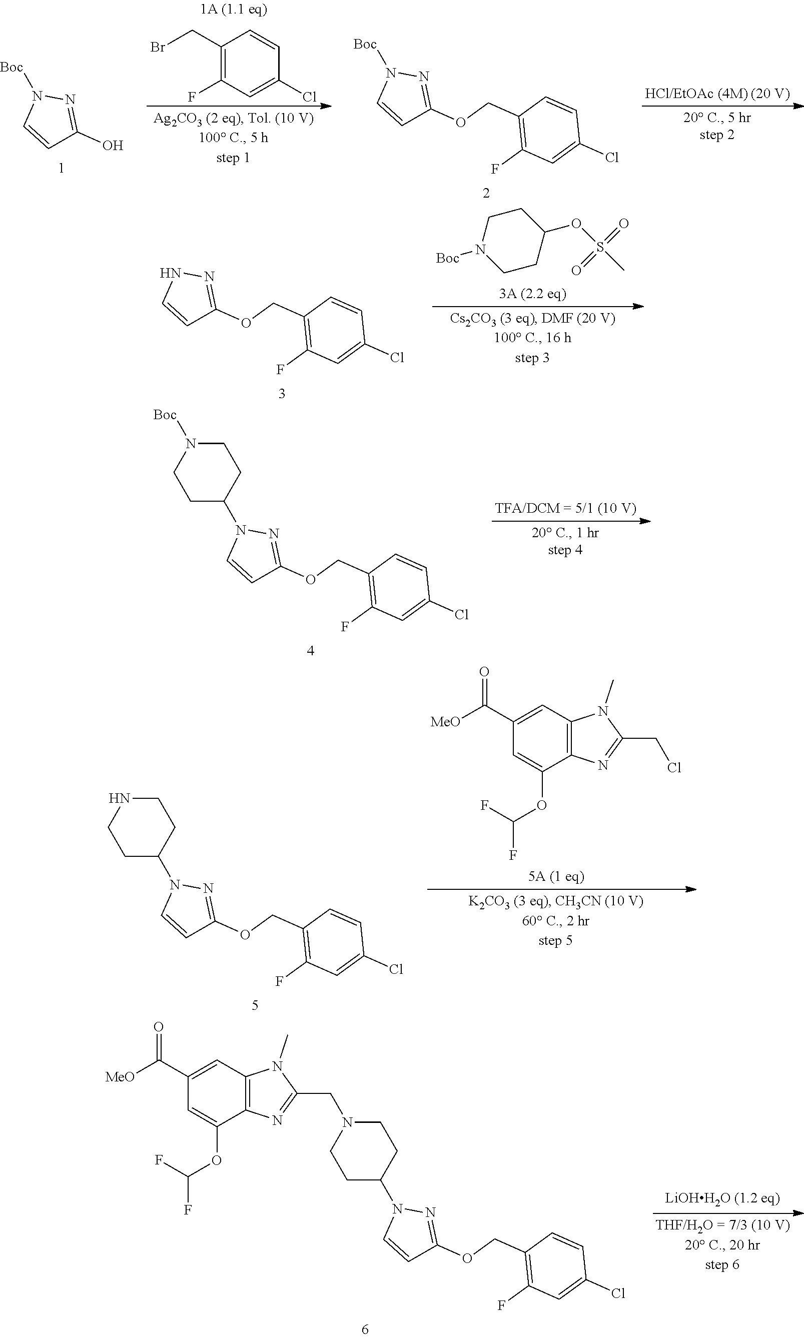

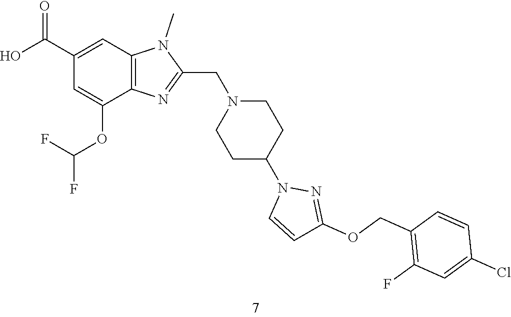 [SMILES: Cn1c(CN2CCC(n3ccc(OCc4ccc(Cl)cc4F)n3)CC2)nc2c(OC(F)F)cc(C(=O)O)cc21]

### General Procedure for Preparation of Intermediate 2:

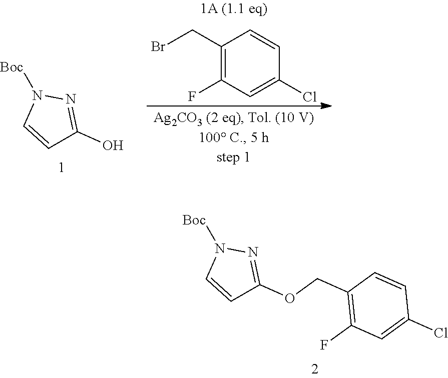 [SMILES: CC(C)(C)OC(=O)n1ccc(O)n1 ; Fc1cc(Cl)ccc1CBr ; CC(C)(C)OC(=O)n1ccc(OCc2ccc(Cl)cc2F)n1]

To a mixture of 1A (2.00 g, 8.96 mmol, 1.1 eq) and 1 (1.5 g, 8.14 mmol, 1 eq) in Toluene (15 mL) was added Ag₂CO₃ (4.49 g, 16.29 mmol, 739.04 μL, 2 eq) in one portion at 20° C. under N₂. The mixture was stirred at 100° C. and stirred for 5 hours. LC-MS (product: RT=0.899 min) showed 1 was consumed completely and one main peak with desired mass was detected. The mixture was filtered and concentrated in vacuum. The residue was purified by column chromatography (SiO₂, PE/EtOAc=10/1 to 0/1). 2 (1.7 g, 5.20 mmol, 63.89% yield) was obtained as colorless oil.

LCMS: RT=0.899 min, MS cal.: 326.1, [M−(t-Bu)+H]⁺=271.0

¹H NMR (400 MHz, MeOH-d4) δ=8.02 (d, J=3.2 Hz, 1H) 7.56 (t, J=8.0 Hz, 1H) 7.23-7.30 (m, 2H) 6.05 (d, J=3.2 Hz, 1H) 5.32 (s, 2H) 1.64 (s, 9H)

### General Procedure for Preparation of Intermediate 3:

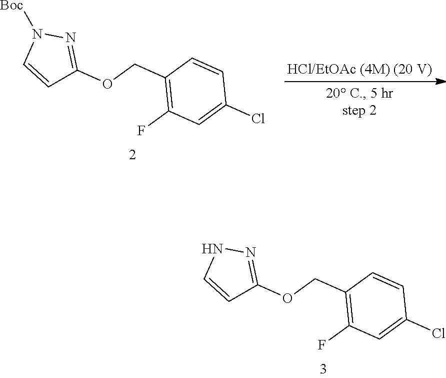 [SMILES: CC(C)(C)OC(=O)n1ccc(OCc2ccc(Cl)cc2F)n1 ; Fc1cc(Cl)ccc1COc1cc[nH]n1]

To a mixture of 2 (1.7 g, 5.20 mmol, 1 eq) in EtOAc (2 mL) was added HCl/EtOAc (4 M, 20 mL, 15.38 eq) at 20° C. The mixture was stirred at 20° C. for 5 hours. LC-MS (product: RT=0.455 min) showed 2 was consumed completely and one main peak with desired mass was detected. The reaction mixture was quenched by saturated NaHCO₃ solution to pH=8. The aqueous phase was extracted with DCM (10 mL*3). The combined organic phase was washed with brine (10 mL*3), dried with anhydrous Na₂SO₄, filtered, and concentrated in vacuum. The residue was purified by column chromatography (SiO₂, PE/EtOAc=99/1 to 88/12). 3 (1.02 g, 4.50 mmol, 86.50% yield) was obtained as light-yellow oil.

LCMS: RT=0.455 min, MS cal.: 226.0, [M+H]⁺=227.1

¹H NMR (400 MHz, MeOH-d4) δ=7.51 (t, J=8.4 Hz, 1H) 7.45 (d, J=2.4 Hz, 1H) 7.22-7.25 (m, 1H) 7.20-7.22 (m, 1H) 5.77 (d, J=2.4 Hz, 1H) 5.18 (s, 2H)

### General Procedure for Preparation of Intermediate 4:

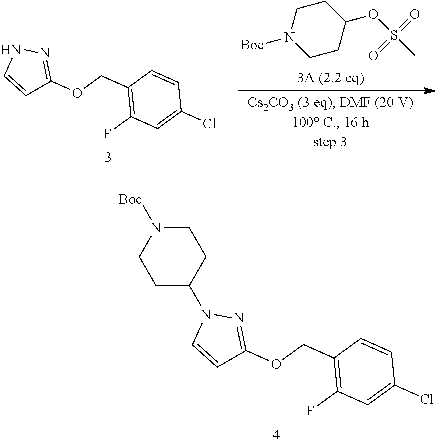 [SMILES: Fc1cc(Cl)ccc1COc1cc[nH]n1 ; CC(C)(C)OC(=O)N1CCC(OS(C)(=O)=O)CC1 ; CC(C)(C)OC(=O)N1CCC(n2ccc(OCc3ccc(Cl)cc3F)n2)CC1]

To a mixture of 3 (300 mg, 1.32 mmol, 1 eq) and 3A (813.52 mg, 2.91 mmol, 2.2 eq) in DMF (6 mL) was added Cs₂CO₃ (1.29 g, 3.97 mmol, 3 eq). The mixture was stirred at 100° C. for 16 hours. LC-MS (product: RT=0.615 min) showed 3 was consumed completely and one main peak with desired mass was detected. The reaction mixture was quenched by addition H₂O (10 mL). The residue was extracted with EtOAc (10 mL*3). The combined organic layers were washed with brine (20 mL*2), dried over Na₂SO₄, filtered, and concentrated in vacuum. The residue was purified by column chromatography (SiO₂, PE/EtOAc=99/1 to 92/8). 4 (273 mg, 666.05 μmol, 50.32% yield) was obtained as a colorless oil.

LCMS: RT=0.615 min, MS cal.: 409.2, [M−(t-Bu)+H]⁺=354.1

¹H NMR (400 MHz, MeOH-d4) δ=7.51 (t, J=8.4 Hz, 1H) 7.45 (d, J=2.4 Hz, 1H) 7.19-7.25 (m, 2H) 5.71 (d, J=2.4 Hz, 1H) 5.17 (s, 2H) 4.06-4.24 (m, 3H) 2.82-3.04 (m, 2H) 2.01 (br d, J=10.4 Hz, 2H) 1.77-1.92 (m, 2H) 1.48 (s, 9H)

### General Procedure for Preparation of Intermediate 5:

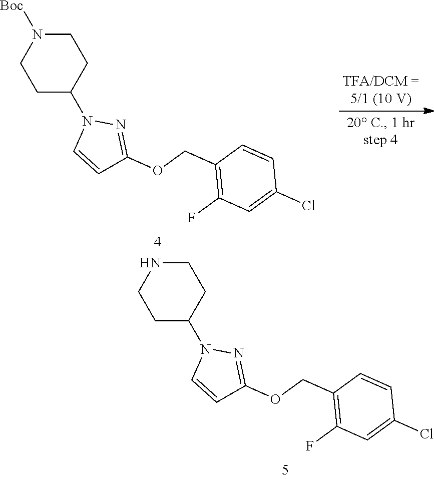

To a mixture of 4 (273 mg, 666.05 μmol, 1 eq) in DCM (5 mL) was added TFA (1.54 g, 13.46 mmol, 1 mL, 20.21 eq). The mixture was stirred at 20° C. for 1 hour. LC-MS (product: RT=0.374 min) showed 4 was consumed completely and one main peak with desired mass was detected. The mixture was concentrated in vacuum. The mixture was added 10 mL DCM. Then the reaction mixture was quenched by NaHCO₃ solution to pH-8. The aqueous phase was extracted with DCM (10 mL*3). The combined organic phase was washed with brine (10 mL*3), dried with anhydrous Na₂SO₄, filtered and concentrated in vacuum. The crude product was used into the next step without further purification. 5 (201 mg, 648.88 μmol, 97.42% yield) was obtained as a white solid.

LCMS: RT=0.374 min, MS cal.: 309.1, [M+H]⁺=310.2

¹H NMR (400 MHz, MeOH-d4) δ=7.51 (t, J=8.4 Hz, 1H) 7.45 (d, J=2.4 Hz, 1H) 7.22 (dt, J=10.0, 2.4 Hz, 2H) 5.73 (d, J=2.4 Hz, 1H) 5.18 (s, 2H) 4.16 (tt, J=11.2, 4.0 Hz, 1H) 3.25-3.30 (m, 2H) 2.87 (td, J=12.8, 2.8 Hz, 2H) 2.06-2.17 (m, 2H) 1.92-2.04 (m, 2H)

### General Procedure for Preparation of Intermediate 6:

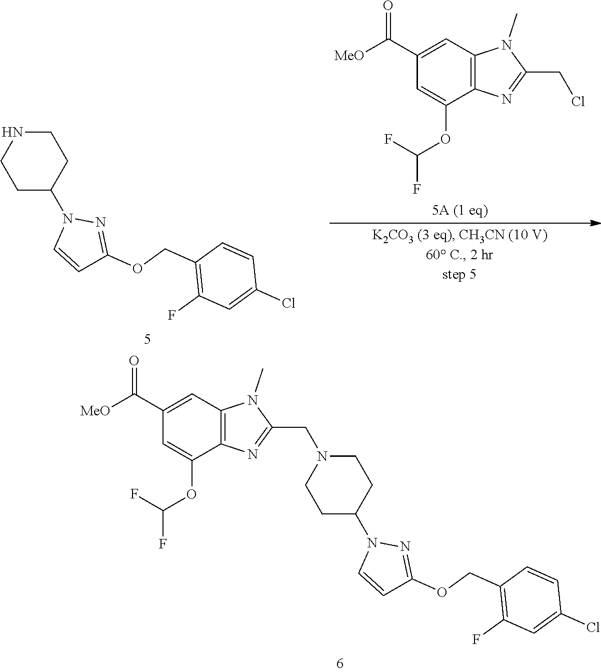 [SMILES: Fc1cc(Cl)ccc1COc1ccn(C2CCNCC2)n1 ; COC(=O)c1cc(OC(F)F)c2nc(CCl)n(C)c2c1 ; COC(=O)c1cc(OC(F)F)c2nc(CN3CCC(n4ccc(OCc5ccc(Cl)cc5F)n4)CC3)n(C)c2c1]

To a mixture of 5 (80 mg, 258.26 μmol, 1 eq) and 5A (78.69 mg, 258.26 μmol, 1 eq) in ACN (1 mL) was added K₂CO₃ (107.08 mg, 774.78 μmol, 3 eq). The mixture was stirred at 60° C. for 2 hours. LC-MS (product: RT=0.464 min) showed 5 was consumed completely and one main peak with desired mass was detected. The mixture was concentrated in vacuum. The residue was purified by prep-TLC (SiO₂, PE/EtOAc=0/1). 6 (121 mg, 209.35 μmol, 81.06% yield) was obtained as a white solid.

LCMS: RT=0.464 min, MS cal.: 677.2, [M+H]⁺=678.3

¹H NMR (400 MHz, MeOH-d4) δ=8.13 (d, J=1.2 Hz, 1H) 7.67 (s, 1H) 7.50 (t, J=8.4 Hz, 1H) 7.44 (d, J=2.4 Hz, 1H) 7.26 (t, J=74.4 Hz, 1H) 7.18-7.21 (m, 2H) 5.70 (d, J=2.4 Hz, 1H) 5.16 (s, 2H) 4.02 (s, 3H) 3.99 (br d, J=8.0 Hz, 1H) 3.96 (s, 3H) 3.92 (s, 2H) 3.00 (br d, J=12.0 Hz, 2H) 2.28-2.43 (m, 2H) 1.95-2.09 (m, 4H)

### General Procedure for Preparation of Compound 217 (7):

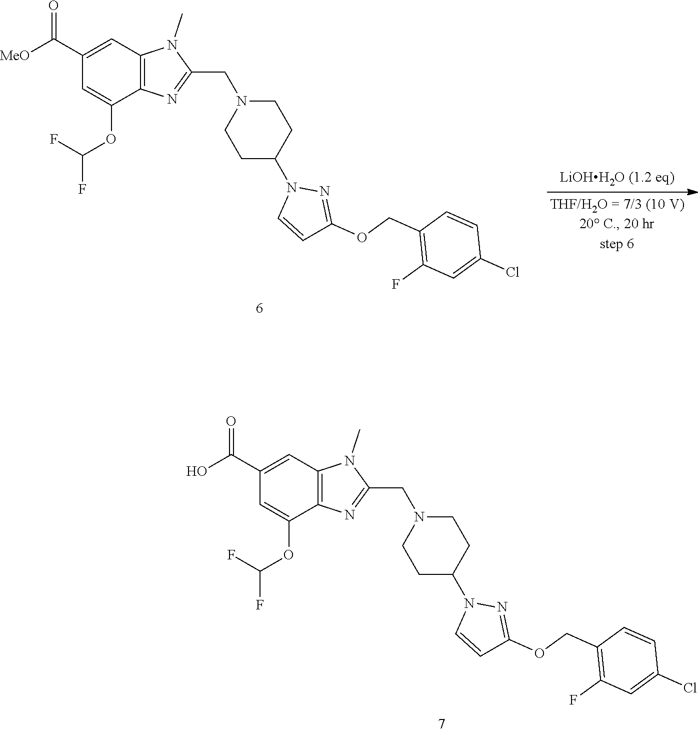 [SMILES: COC(=O)c1cc(OC(F)F)c2nc(CN3CCC(n4ccc(OCc5ccc(Cl)cc5F)n4)CC3)n(C)c2c1 ; Cn1c(CN2CCC(n3ccc(OCc4ccc(Cl)cc4F)n3)CC2)nc2c(OC(F)F)cc(C(=O)O)cc21]

To a mixture of 6 (121 mg, 209.35 μmol, 1 eq) in THF (2.1 mL) and H₂O (0.9 mL) was added LiOH·H₂O (10.54 mg, 251.22 μmol, 1.2 eq). The mixture was stirred at 20° C. for 20 hours. LC-MS (product: RT=1.564 min) showed 6 was consumed completely and one main peak with desired mass was detected. The mixture was concentrated in vacuum. The residue was purified by prep-HPLC (column: Waters xbridge 150*25 mm 10 um; mobile phase: [H₂O (10 mM NH₄HCO₃)-ACN]; gradient: 30%-60% B over 9.0 min). 2-((4-(3-((4-Chloro-2-fluorobenzyl)oxy)-1H-pyrazol-1-yl)piperidin-1-yl)methyl)-4-(difluoromethoxy)-1-methyl-1H-benzo[d]imidazole-6-carboxylic acid (28.39 mg, 50.13 μmol, 23.95% yield) was obtained as a white solid.

LCMS: RT=2.574 min, MS cal.: 563.2, [M+H]⁺=564.2

HPLC: RT=11.133 min, purity: 99.59%

¹H NMR (400 MHz, DMSO-d₆) δ=8.05 (s, 1H) 7.61 (t, J=74.8 Hz, 1H) 7.56-7.60 (m, 2H) 7.54 (d, J=3.6 Hz, 1H) 7.47 (dd, J=10.0, 2.0 Hz, 1H) 7.31 (dd, J=8.4, 1.6 Hz, 1H) 5.68 (d, J=2.4 Hz, 1H) 5.12 (s, 2H) 3.92-4.03 (m, 4H) 3.86 (s, 2H) 2.87-2.98 (m, 2H) 2.26 (br t, J=10.4 Hz, 2H) 1.80-1.99 (m, 4H)

### Example A141—Preparation of Compound 221. 4-(Difluoromethoxy)-2-((4-(6-((4-(difluoromethoxy)-2-fluorobenzyl)oxy)pyridin-2-yl)piperidin-1-yl)methyl)-1-methyl-1H-benzo[d]imidazole-6-carboxylic acid (Compound 221) (5)

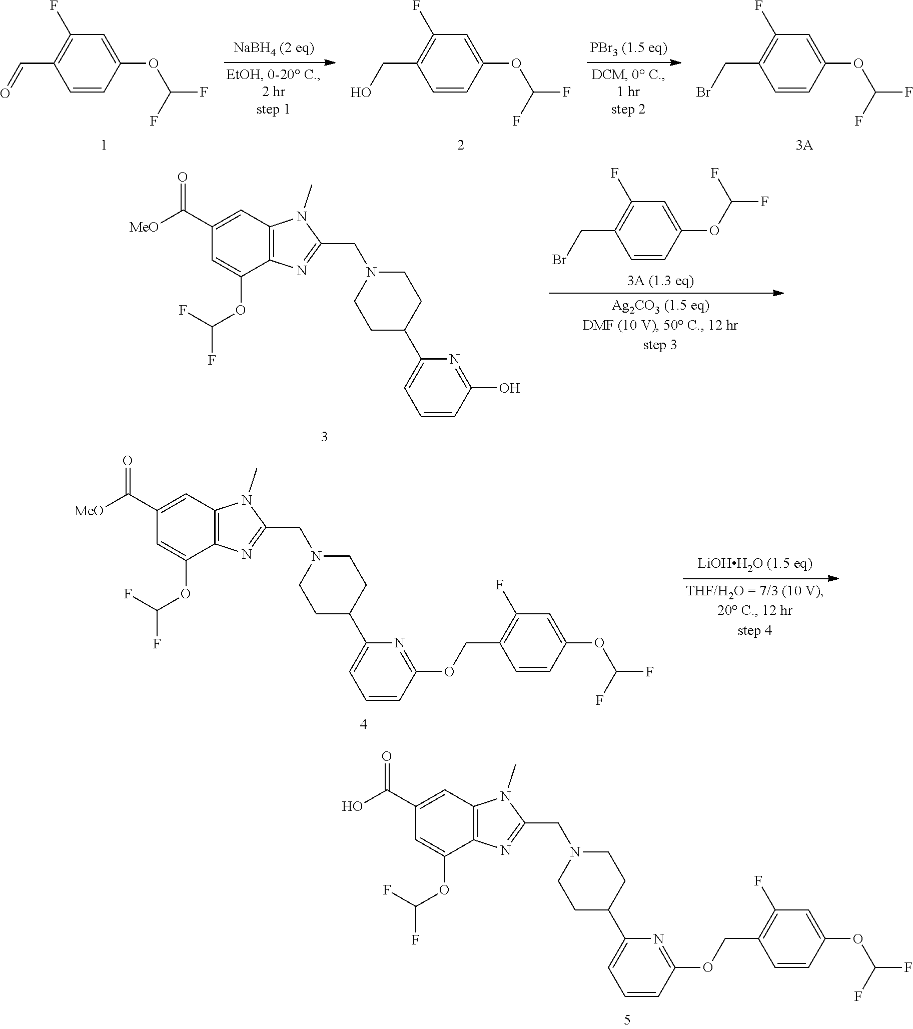

### General Procedure for Preparation of Intermediate 2:

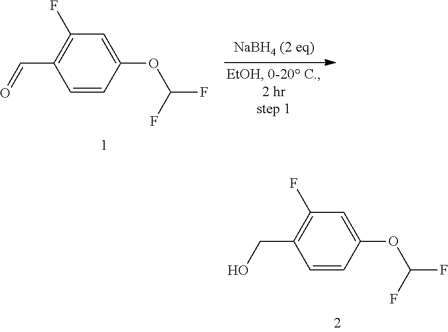 [SMILES: O=Cc1ccc(OC(F)F)cc1F ; OCc1ccc(OC(F)F)cc1F]

To a solution of 1 (4.7 g, 24.72 mmol, 1 eq) in EtOH (50 mL) was added NaBH₄ (1.87 g, 49.44 mmol, 2 eq) at 0° C. The mixture was stirred at 20° C. for 2 hr under N₂ atmosphere. TLC (PE/EA=1/1, R_{f}=0.3) indicated 1 was consumed completely and one new spot was formed. The reaction was clean according to TLC. The reaction mixture was added to H₂O (100 mL) at 25° C. The mixture was extracted by EA (50 mL*3). Then organic phase was combined and washed by brine (50 mL), dried over Na₂SO₄, filtered, and concentrated under reduced pressure to give a residue. The crude product was not purified and used to next step directly. 2 (4.4 g, 22.90 mmol, 92.63% yield) was obtained as a colorless liquid.

¹H NMR (400 MHz, CHCl3-d) δ=7.44 (t, J=8.0 Hz, 1H), 6.94 (dd, J=1.6, 2.0 Hz, 1H), 6.88 (dd, J=2.4, 2.0 Hz, 1H), 6.51 (t, J=73.6 Hz, 1H), 4.75 (s, 2H)

### General Procedure for Preparation of Intermediate 3A:

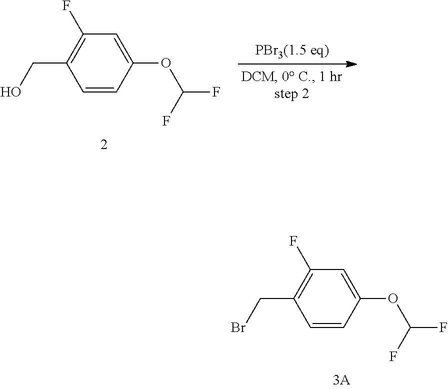 [SMILES: OCc1ccc(OC(F)F)cc1F ; Fc1cc(OC(F)F)ccc1CBr]

To a solution of 2 (500 mg, 2.60 mmol, 1 eq) in DCM (10 mL) was added PBr₃ (1.06 g, 3.90 mmol, 1.5 eq) at 0° C. The mixture was stirred at 0° C. for 1 h under N₂ atmosphere. TLC (PE/EA=5/1, R_{f}=0.7) indicated 2 was consumed completely and one new spot was formed. The reaction was clean according to TLC. The reaction mixture was quenched by addition NaHCO₃ (5 mL) and extracted with DCM (5 mL*3). The combined organic layers were washed with NaCl (5 mL), dried over Na₂SO₄, filtered and concentrated under reduced pressure to give a residue. The crude product was not purified and used to next step directly. 3A (250 mg, 980.27 μmol, 37.67% yield) was obtained as a light-yellow liquid.

¹H NMR (400 MHz, CHCl3-d) δ=7.40 (t, J=8.4 Hz, 1H), 6.97-6.85 (m, 2H), 6.53 (t, J=73.2 Hz, 1H), 4.50 (s, 2H)

### General Procedure for Preparation of Intermediate 4:

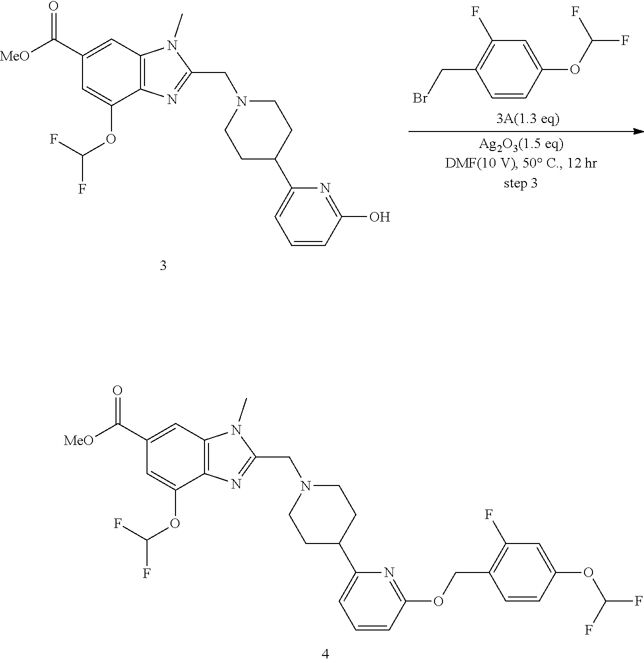 [SMILES: COC(=O)c1cc(OC(F)F)c2nc(CN3CCC(c4cccc(O)n4)CC3)n(C)c2c1 ; Fc1cc(OC(F)F)ccc1CBr ; COC(=O)c1cc(OC(F)F)c2nc(CN3CCC(c4cccc(OCc5ccc(OC(F)F)cc5F)n4)CC3)n(C)c2c1]

To a solution of 3 (120 mg, 268.79 μmol, 1 eq) and 3A (89.11 mg, 349.43 μmol, 1.3 eq) in DMF (4 mL) was added Ag₂CO₃ (111.18 mg, 403.18 μmol, 1.5 eq). The mixture was stirred at 50° C. for 12 hr under N₂ atmosphere. LCMS showed 3 was consumed completely and one main peak with desired mass was detected. The mixture was filtered and added water (10 mL), extracted with EA (10 mL*3), the organic phase was washed with NaCl (20 mL), filtered, and concentrated in vacuum to get a residue. The residue was purified by prep-TLC (SiO₂, PE:EA=0:1). 4 (135 mg, 217.54 μmol, 64.7% yield) as a yellow solid.

LCMS: RT=0.504 min, MS cal.: 620.2, [M+H]⁺=621.2

¹H NMR (400 MHz, DMSO-d₆) δ=8.11 (d, J=0.8 Hz, 1H), 7.84 (s, 1H), 7.65 (t, J=74.4, 1H), 7.64-7.56 (m, 2H), 7.53 (s, 1H), 7.46 (s, 1H), 7.28 (t, J=73.6, 1H), 7.15 (dd, J=2.4, 2.4 Hz, 1H), 7.10 (s, 1H), 7.02 (dd, J=2.0, 2.0 Hz, 1H), 6.86 (d, J=7.6 Hz, 1H), 6.65 (d, J=8.0 Hz, 1H), 5.35 (s, 2H), 3.98 (s, 3H), 3.90 (s, 3H), 3.86 (s, 2H), 2.95 (br d, J=11.2 Hz, 2H), 2.65-2.54 (m, 1H), 2.23 (br t, J=10.0 Hz, 2H), 1.84-1.65 (m, 4H)

### General Procedure for Preparation of Compound 221 (5)

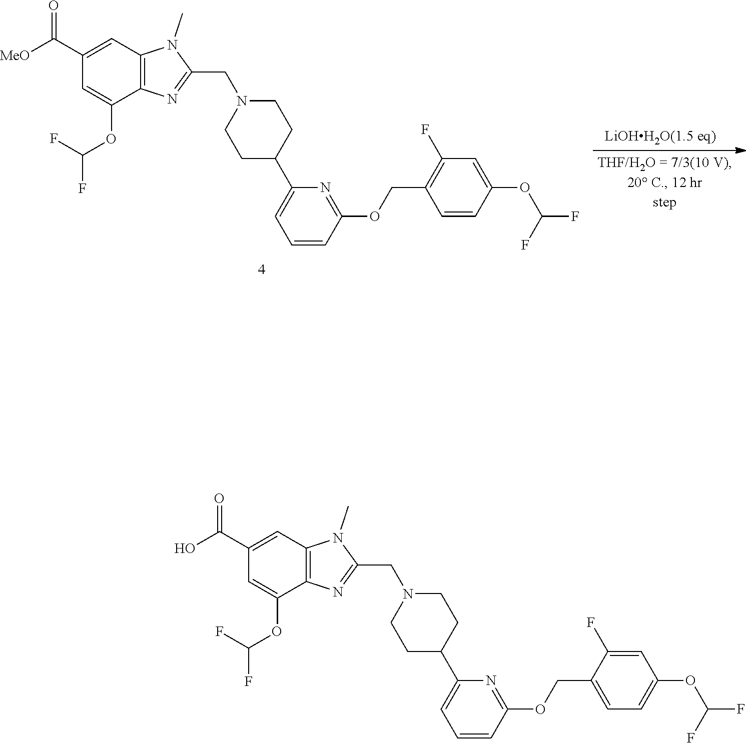 [SMILES: COC(=O)c1cc(OC(F)F)c2nc(CN3CCC(c4cccc(OCc5ccc(OC(F)F)cc5F)n4)CC3)n(C)c2c1 ; Cn1c(CN2CCC(c3cccc(OCc4ccc(OC(F)F)cc4F)n3)CC2)nc2c(OC(F)F)cc(C(=O)O)cc21]

To a solution of 4 (115 mg, 185.31 μmol, 1 eq) in THF (1.4 mL) was added LiOH·H₂O (11.66 mg, 277.97 μmol, 1.5 eq) in H₂O (0.6 mL). The mixture was stirred at 20° C. for 12 hr. LCMS showed 4 was consumed completely and one main peak with desired mass was detected. The mixture was filtered and filtrate was concentrated in vacuum to get a residue. The residue was purified by prep-HPLC (column: Waters Xbridge BEH C18 100*30 mm*10 um; mobile phase: [H₂O (10 mM NH₄HCO₃)-ACN]; gradient: 25%-70% B over 8.0 min). 4-(Difluoromethoxy)-2-((4-(6-((4-(difluoromethoxy)-2-fluorobenzyl)oxy)pyridin-2-yl)piperidin-1-yl)methyl)-1-methyl-1H-benzo[d]imidazole-6-carboxylic acid (28.72 mg, 47.35 μmol, 21.77% yield) was obtained as a white solid.

LCMS: RT=2.434 min, MS cal.: 606.2, [M+H]⁺=607.2

HPLC: purity: 99.2%

¹H NMR (400 MHz, DMSO-d₆) δ=13.62-12.57 (m, 1H), 8.06 (d, J=0.8 Hz, 1H), 7.62 (t, J=74.4 Hz, 1H), 7.64-7.57 (m, 2H), 7.52 (s, 1H), 7.28 (t, J=73.6 Hz, 1H), 7.15 (dd, J=2.0, 2.0 Hz, 1H), 7.10 (s, 1H), 7.02 (dd, J=2.4, 2.4 Hz, 1H), 6.86 (d, J=7.2 Hz, 1H), 6.65 (d, J=8.4 Hz, 1H), 5.35 (s, 2H), 3.96 (s, 3H), 3.85 (s, 2H), 2.95 (br d, J=11.2 Hz, 2H), 2.63-2.56 (m, 1H), 2.28-2.17 (m, 2H), 1.85-1.66 (m, 4H)

### Example A142—Preparation of Compound 222. 4-(Difluoromethoxy)-2-((4-(6-((4-(difluoromethoxy)-2-fluorobenzyl)oxy)pyridin-2-yl)piperidin-1-yl)methyl)-1-methyl-1H-benzo[d]imidazole-6-carboxylic acid (Compound 222) (3)

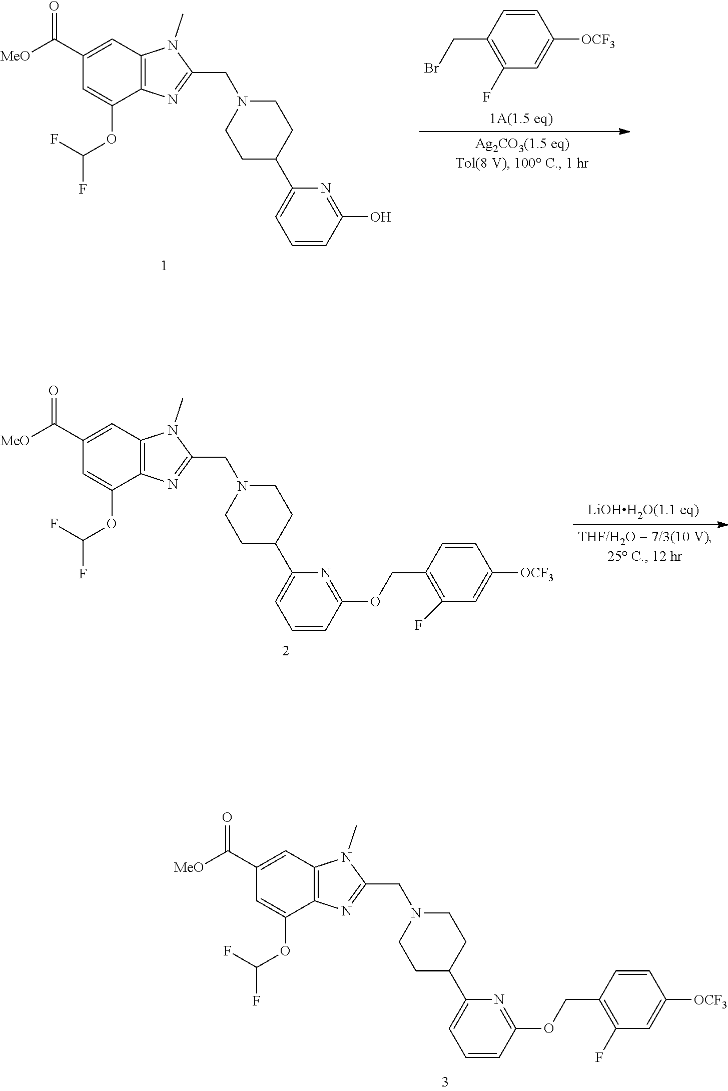 [SMILES: COC(=O)c1cc(OC(F)F)c2nc(CN3CCC(c4cccc(O)n4)CC3)n(C)c2c1 ; Fc1cc(OC(F)(F)F)ccc1CBr ; COC(=O)c1cc(OC(F)F)c2nc(CN3CCC(c4cccc(OCc5ccc(OC(F)(F)F)cc5F)n4)CC3)n(C)c2c1 ; COC(=O)c1cc(OC(F)F)c2nc(CN3CCC(c4cccc(OCc5ccc(OC(F)(F)F)cc5F)n4)CC3)n(C)c2c1]

### General Procedure for Preparation of Intermediate 2:

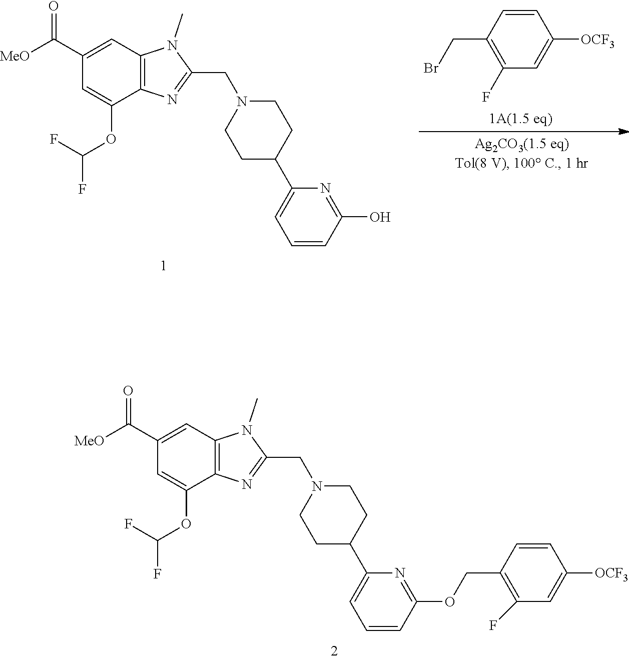 [SMILES: COC(=O)c1cc(OC(F)F)c2nc(CN3CCC(c4cccc(O)n4)CC3)n(C)c2c1 ; Fc1cc(OC(F)(F)F)ccc1CBr ; COC(=O)c1cc(OC(F)F)c2nc(CN3CCC(c4cccc(OCc5ccc(OC(F)(F)F)cc5F)n4)CC3)n(C)c2c1]

A mixture of 1 (120 mg, 268.79 μmol, 1 eq), 1A (110.08 mg, 403.18 μmol, 1.5 eq), Ag₂CO₃ (111.18 mg, 403.18 μmol, 18.29 μL, 1.5 eq) in Toluene (1 mL) was degassed and purged with N₂ for 3 times, and then the mixture was stirred at 100° C. for 1 hr under N₂ atmosphere. LC-MS (product, Rt=1.860 min) showed 1 was consumed completely and desired mass was detected. The suspension was filtered through a pad of Celite, and filter cake was washed with EtOAc (10 mL*3). The reaction mixture was concentrated under reduced pressure to give a residue. The residue was purified by column chromatography (SiO₂, Petroleum ether/Ethyl acetate=10/1 to 1/1). 2 (60 mg, 93.96 μmol, 34.96% yield) was obtained as a yellow solid.

LCMS: RT=1.860 min, MS cal.: 638.20, [M+H]⁺=639.3

¹H NMR (400 MHz, CDCl₃) δ=7.99 (s, 1H), 7.72 (s, 1H), 7.56 (s, 1H), 7.55-7.49 (m, 2H), 7.32 (t, J=74.4 Hz, 1H), 6.99 (br t, J=10.4 Hz, 2H), 6.75 (d, J=7.2 Hz, 1H), 6.62 (d, J=8.0 Hz, 1H), 5.43 (s, 2H), 4.05-3.94 (m, 7H), 2.99 (br s, 2H), 2.64 (br s, 1H), 2.41-2.24 (m, 2H), 1.97-1.80 (m, 4H)

### General Procedure for Preparation of Compound 222 (3):

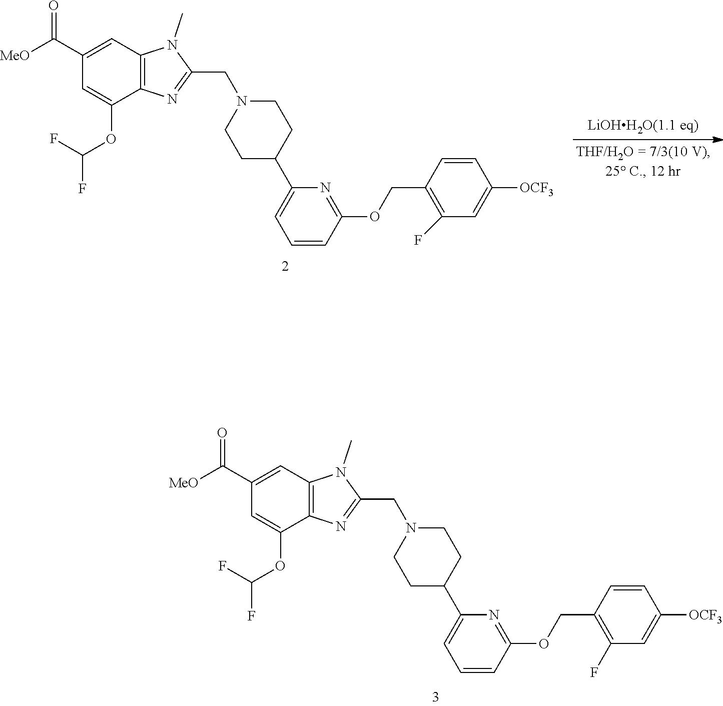 [SMILES: COC(=O)c1cc(OC(F)F)c2nc(CN3CCC(c4cccc(OCc5ccc(OC(F)(F)F)cc5F)n4)CC3)n(C)c2c1 ; COC(=O)c1cc(OC(F)F)c2nc(CN3CCC(c4cccc(OCc5ccc(OC(F)(F)F)cc5F)n4)CC3)n(C)c2c1]

A mixture of 2 (50 mg, 78.30 μmol, 1 eq), LiOH·H₂O (3.61 mg, 86.13 μmol, 1.1 eq) in THF (0.7 mL) and H₂O (0.3 mL) was degassed and purged with N₂ for 3 times, and then the mixture was stirred at 25° C. for 12 hr under N₂ atmosphere. LC-MS (product RT=1.305 min) showed was 2 consumed completely and one main peak with desired mass was detected. The reaction was purified directly. The residue was purified by prep-HPLC (column: Waters Xbridge Prep OBD C18 150*40 mm*10 um; mobile phase: [H₂O (10 mM NH₄HCO₃)-ACN]; gradient: 15%-65% B over 8.0 min). 4-(Difluoromethoxy)-2-((4-(6-((4-(difluoromethoxy)-2-fluorobenzyl)oxy)pyridin-2-yl)piperidin-1-yl)methyl)-1-methyl-1H-benzo[d]imidazole-6-carboxylic acid (28.12 mg, 44.92 μmol, 57.37% yield, 99.77% purity) was obtained as a white solid.

LCMS: RT=2.593 min, MS cal.: 624.18, [M+H]⁺=625.3

HPLC: RT=12.90 min, purity: 99.77%

¹H NMR (400 MHz, DMSO) δ=8.06 (br s, 1H), 7.67 (s, 1H), 7.66-7.61 (m, 1H), 7.69-7.59 (m, 1H), 7.63 (t, J=74.4 Hz, 1H), 7.60 (s, 1H), 7.51 (s, 1H), 7.40 (br d, J=10.0 Hz, 1H), 7.22 (br d, J=9.2 Hz, 1H), 6.86 (d, J=7.2 Hz, 1H), 6.68 (d, J=8.0 Hz, 1H), 5.39 (s, 2H), 3.96 (s, 3H), 3.84 (s, 2H), 2.92 (br d, J=10.4 Hz, 2H), 2.60-2.55 (m, 1H), 2.25-2.16 (m, 2H), 1.82-1.60 (m, 4H)

### Example A143—Preparation of Compound 223. 4-(Difluoromethoxy)-2-((4-(6-((2-methoxy-4-(trifluoromethyl)benzyl)oxy)pyridin-2-yl)piperidin-1-yl)methyl)-1-methyl-1H-benzo[d]imidazole-6-carboxylic acid (Compound 223) (3)

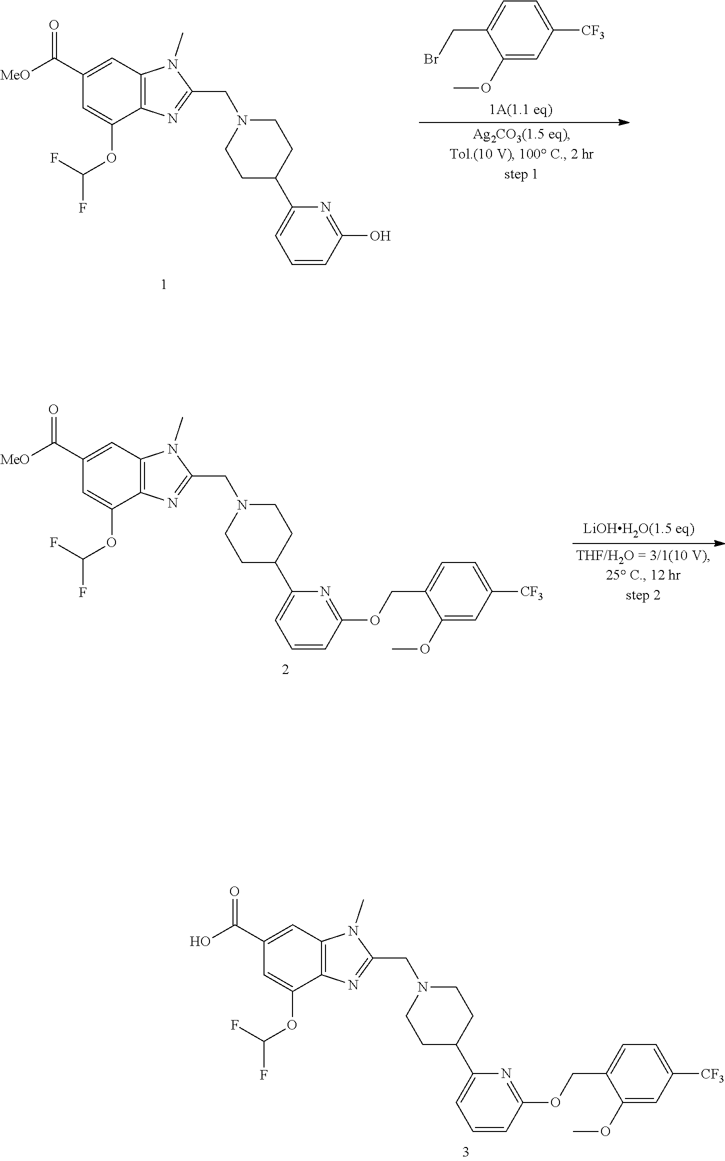 [SMILES: COC(=O)c1cc(OC(F)F)c2nc(CN3CCC(c4cccc(O)n4)CC3)n(C)c2c1 ; COc1cc(C(F)(F)F)ccc1CBr ; COC(=O)c1cc(OC(F)F)c2nc(CN3CCC(c4cccc(OCc5ccc(C(F)(F)F)cc5OC)n4)CC3)n(C)c2c1 ; COc1cc(C(F)(F)F)ccc1COc1cccc(C2CCN(Cc3nc4c(OC(F)F)cc(C(=O)O)cc4n3C)CC2)n1]

### General Procedure for Preparation of Intermediate 2:

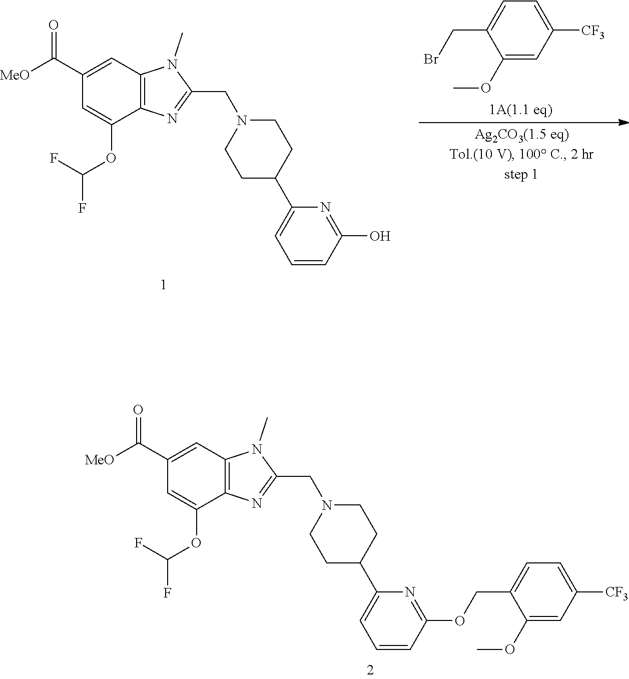 [SMILES: COC(=O)c1cc(OC(F)F)c2nc(CN3CCC(c4cccc(O)n4)CC3)n(C)c2c1 ; COc1cc(C(F)(F)F)ccc1CBr ; COC(=O)c1cc(OC(F)F)c2nc(CN3CCC(c4cccc(OCc5ccc(C(F)(F)F)cc5OC)n4)CC3)n(C)c2c1]

To a solution of 1 (200 mg, 447.98 μmol, 1 eq), 1A (126.66 mg, 492.78 μmol, 1.1 eq) in Toluene (3 mL) was added Ag₂CO₃ (185.29 mg, 671.97 μmol, 30.49 μL, 1.5 eq). The mixture was stirred at 100° C. for 2 hr. LC-MS showed 1 was consumed completely and desired mass was detected. The mixture was diluted with H₂O (20 mL) and extracted with EtOAc (30 mL). The combined organic layers were washed with brine (20 mL), dried over Na₂SO₄, filtered, and concentrated under reduced pressure to give a residue. The residue was purified by column chromatography (SiO₂, Petroleum ether/Ethyl acetate=1/1 to 0/1). 2 (100 mg, 160.63 μmol, 35.86% yield) was obtained as a white solid.

LCMS: RT=1.749 min, MS cal.: 622.56, [M+H]⁻=623.1

¹H NMR (400 MHz, CHCl3-d) δ=7.99 (s, 1H), 7.72 (s, 1H), 7.64 (br t, J=7.6 Hz, 1H), 7.56-7.48 (m, 1H), 7.40 (d, J=8.0 Hz, 1H), 7.34 (d, J=10.8 Hz, 1H), 7.32 (t, J=74.4 Hz, 1H), 6.75 (d, J=7.2 Hz, 1H), 6.65 (d, J=8.0 Hz, 1H), 5.50 (s, 2H), 4.01 (s, 3H), 3.97 (s, 4H), 3.89 (br s, 2H), 3.09-2.91 (m, 2H), 2.62 (br s, 1H), 2.30 (br d, J=1.2 Hz, 2H), 1.94-1.70 (m, 4H)

### General Procedure for Preparation of Compound 223 (3):

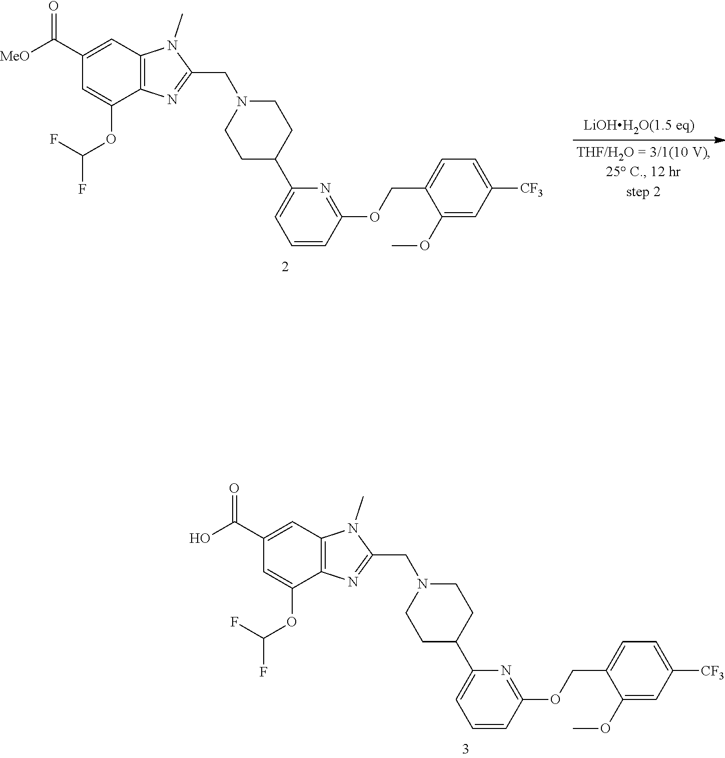 [SMILES: COC(=O)c1cc(OC(F)F)c2nc(CN3CCC(c4cccc(OCc5ccc(C(F)(F)F)cc5OC)n4)CC3)n(C)c2c1 ; COc1cc(C(F)(F)F)ccc1COc1cccc(C2CCN(Cc3nc4c(OC(F)F)cc(C(=O)O)cc4n3C)CC2)n1]

To a solution of 2 (100 mg, 160.63 μmol, 1 eq) in THF (0.7 mL) was added LiOH H₂O (10.11 mg, 240.94 μmol, 1.5 eq) in H₂O (0.3 mL). The mixture was stirred at 25° C. for 12 hr. LC-MS showed 2 was consumed completely and desired mass was detected. The reaction mixture was purified directly. The residue was purified by prep-HPLC (neutral condition; column: Waters Xbridge Prep OBD C18 150*40 mm*10 um; mobile phase: [H₂O (10 mM NH₄HCO₃)-ACN]; gradient: 15%-65% B over 8.0 min). 4-(Difluoromethoxy)-2-((4-(6-((2-methoxy-4-(trifluoromethyl)benzyl)oxy)pyridin-2-yl)piperidin-1-yl)methyl)-1-methyl-1H-benzo[d]imidazole-6-carboxylic acid (28.99 mg, 47.16 μmol, 29.36% yield, 99% purity) was obtained as a white solid.

LCMS: RT=3.262 min, MS cal.: 608.53, [M+H]⁺=609.1

HPLC: RT=12.820 min, purity: 100.0%

¹H NMR (400 MHz, MeOH-d4) δ=8.10 (d, J=1.2 Hz, 1H), 7.72-7.65 (m, 2H), 7.59 (dd, J=7.6, 8.1 Hz, 1H), 7.49-7.41 (m, 2H), 7.24 (t, J=74.4 Hz, 1H), 6.83 (d, J=7.2 Hz, 1H), 6.67 (d, J=8.0 Hz, 1H), 5.50 (s, 2H), 4.01 (s, 3H), 3.97 (s, 2H), 3.07 (br d, J=11.6 Hz, 2H), 2.71-2.60 (m, 1H), 2.45-2.34 (m, 2H), 1.91-1.80 (m, 4H)

### Example A144—Preparation of Compound 224. 2-((4-(6-((4-Chloro-2-fluorobenzyl)oxy)pyridin-2-yl)piperidin-1-yl)methyl)-1-methyl-4-(2,2,2-trifluoroethoxy)-1H-benzo[d]imidazole-6-carboxylic acid (Compound 224) (3)

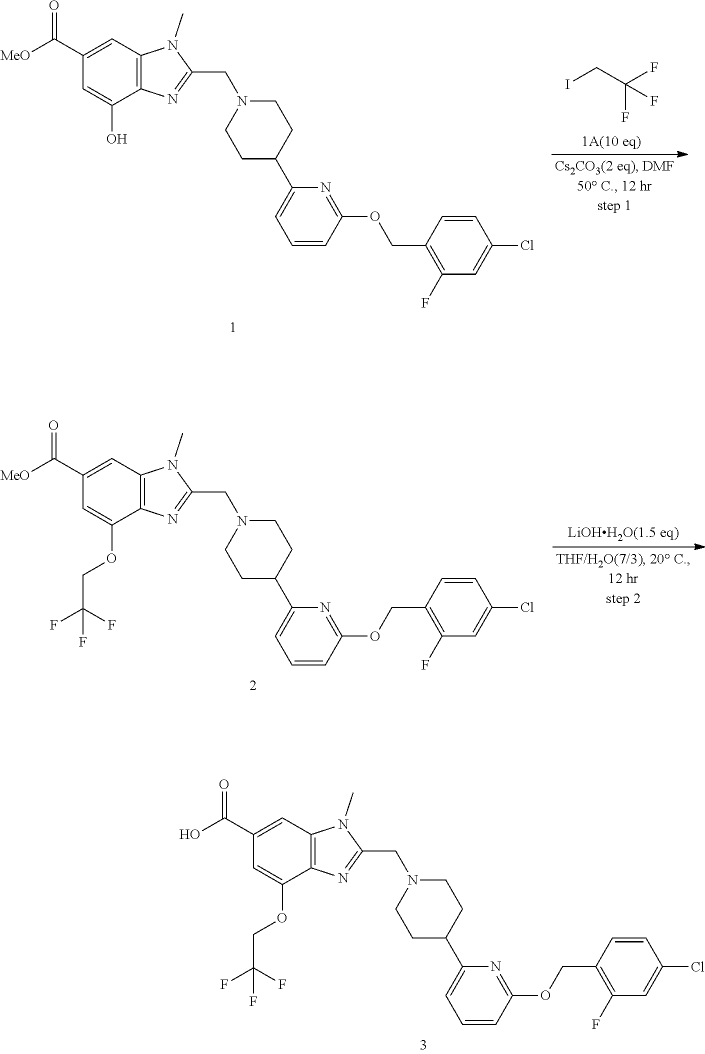 [SMILES: COC(=O)c1cc(O)c2nc(CN3CCC(c4cccc(OCc5ccc(Cl)cc5F)n4)CC3)n(C)c2c1 ; FC(F)(F)CI ; COC(=O)c1cc(OCC(F)(F)F)c2nc(CN3CCC(c4cccc(OCc5ccc(Cl)cc5F)n4)CC3)n(C)c2c1 ; Cn1c(CN2CCC(c3cccc(OCc4ccc(Cl)cc4F)n3)CC2)nc2c(OCC(F)(F)F)cc(C(=O)O)cc21]

### General Procedure for Preparation of Intermediate 2:

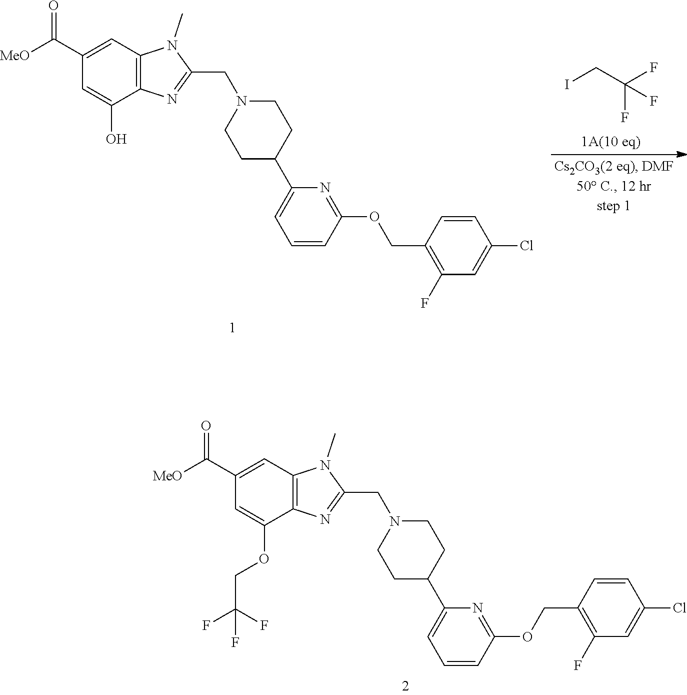 [SMILES: COC(=O)c1cc(O)c2nc(CN3CCC(c4cccc(OCc5ccc(Cl)cc5F)n4)CC3)n(C)c2c1 ; FC(F)(F)CI ; COC(=O)c1cc(OCC(F)(F)F)c2nc(CN3CCC(c4cccc(OCc5ccc(Cl)cc5F)n4)CC3)n(C)c2c1]

To a solution of 1 (60 mg, 111.32 μmol, 1 eq) and 1A (233.70 mg, 1.11 mmol, 109.20 μL, 10 eq) in DMF (1 mL) was added Cs₂CO₃ (72.54 mg, 222.64 μmol, 2 eq). The mixture was stirred at 50° C. for 12 hr in the sealed tube under N₂ atmosphere. LCMS showed 1 was consumed completely and one main peak with desired mass was detected. The reaction mixture was quenched by addition water (5 mL), and then extracted with EA (3 mL*3). The combined organic layers were washed with NaCl (10 mL*2), dried over Na₂SO₄, filtered, and concentrated under reduced pressure to give a residue. The residue was purified by prep-TLC (SiO₂, PE:EA=1:1). 2 (73 mg, 129.87 μmol, 90% yield) was obtained as a yellow solid.

¹H NMR (400 MHz, DMSO-d₆) δ=7.93 (s, 1H), 7.62 (t, J=8.0 Hz, 1H), 7.55 (t, J=8.0 Hz, 1H), 7.45 (dd, J=1.6, 2.0 Hz, 1H), 7.38 (s, 1H), 7.28 (dd, J=1.6, 1.6 Hz, 1H), 6.86 (d, J=7.2 Hz, 1H), 6.66 (d, J=8.0 Hz, 1H), 5.35 (s, 2H), 5.13 (q, J=8.8 Hz, 2H), 3.95 (s, 3H), 3.89 (s, 3H), 3.84 (s, 2H), 2.93 (br d, J=10.8 Hz, 2H), 2.62-2.56 (m, 1H), 2.21 (br t, J=10.4 Hz, 2H), 1.82-1.65 (m, 4H)

### General Procedure for Preparation of Compound 224 (3):

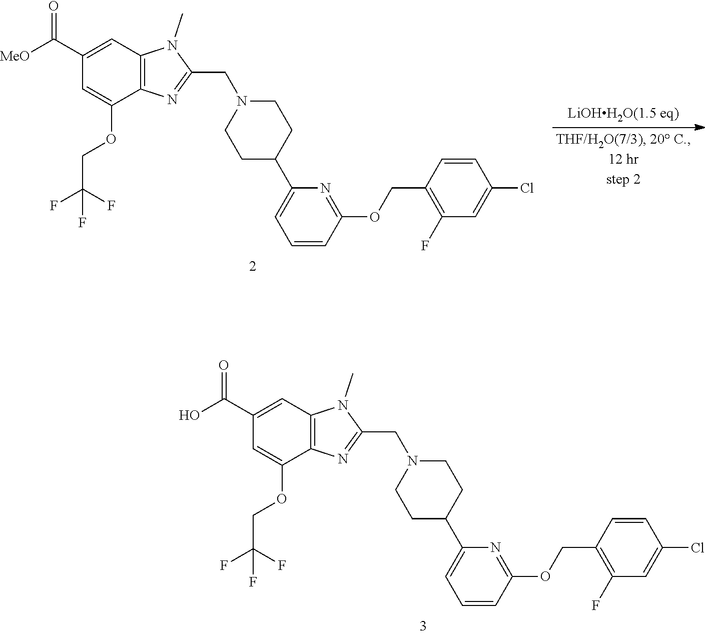 [SMILES: COC(=O)c1cc(OCC(F)(F)F)c2nc(CN3CCC(c4cccc(OCc5ccc(Cl)cc5F)n4)CC3)n(C)c2c1 ; Cn1c(CN2CCC(c3cccc(OCc4ccc(Cl)cc4F)n3)CC2)nc2c(OCC(F)(F)F)cc(C(=O)O)cc21]

To a solution of 2 (63 mg, 101.45 μmol, 1 eq) in THF (1.4 mL) was added LiOH·H₂O (6.39 mg, 152.17 μmol, 1.5 eq) in H₂O (0.6 mL). The mixture was stirred at 20° C. for 12 hr. LCMS showed 2 was consumed completely and one main peak with desired mass was detected. The mixture was filtered, and filtrate was concentrated in vacuum to get a residue. The residue was purified by prep-HPLC (column: 3_Phenomenex Luna C18 75*30 mm*3 um; mobile phase: [H₂O (0.2% FA)-ACN]; gradient: 40%-70% B over 8.0 min). 2-((4-(6-((4-Chloro-2-fluorobenzyl)oxy)pyridin-2-yl)piperidin-1-yl)methyl)-1-methyl-4-(2,2,2-trifluoroethoxy)-1H-benzo[d]imidazole-6-carboxylic acid (30.34 mg, 49.98 μmol, 42.52% yield) was obtained as a light yellow solid.

LCMS: RT=2.511 min, MS cal.: 606.2, [M+1]⁺=607.2

HPLC: purity: 99.4%

¹H NMR (400 MHz, DMSO-d₆) δ=7.89 (d, J=0.8 Hz, 1H), 7.62 (t, J=7.6 Hz, 1H), 7.55 (t, J=8.4 Hz, 1H), 7.45 (dd, J=2.0, 2.0 Hz, 1H), 7.38 (s, 1H), 7.28 (dd, J=2.0, 1.6 Hz, 1H), 6.86 (d, J=7.6 Hz, 1H), 6.66 (d, J=8.4 Hz, 1H), 5.36 (s, 2H), 5.11 (q, J=8.8 Hz, 2H), 3.94 (s, 3H), 3.84 (s, 2H), 2.92 (br d, J=11.2 Hz, 2H), 2.63-2.55 (m, 1H), 2.21 (br t, J=10.0 Hz, 2H), 1.83-1.64 (m, 4H)

### Example A145—Preparation of Compound 225. 2-((4-(6-((4-Chloro-2-fluorobenzyl)oxy)pyridin-2-yl)piperidin-1-yl)methyl)-4-(fluoromethoxy)-1-methyl-1H-benzo[d]imidazole-6-carboxylic acid (Compound 225) (3)

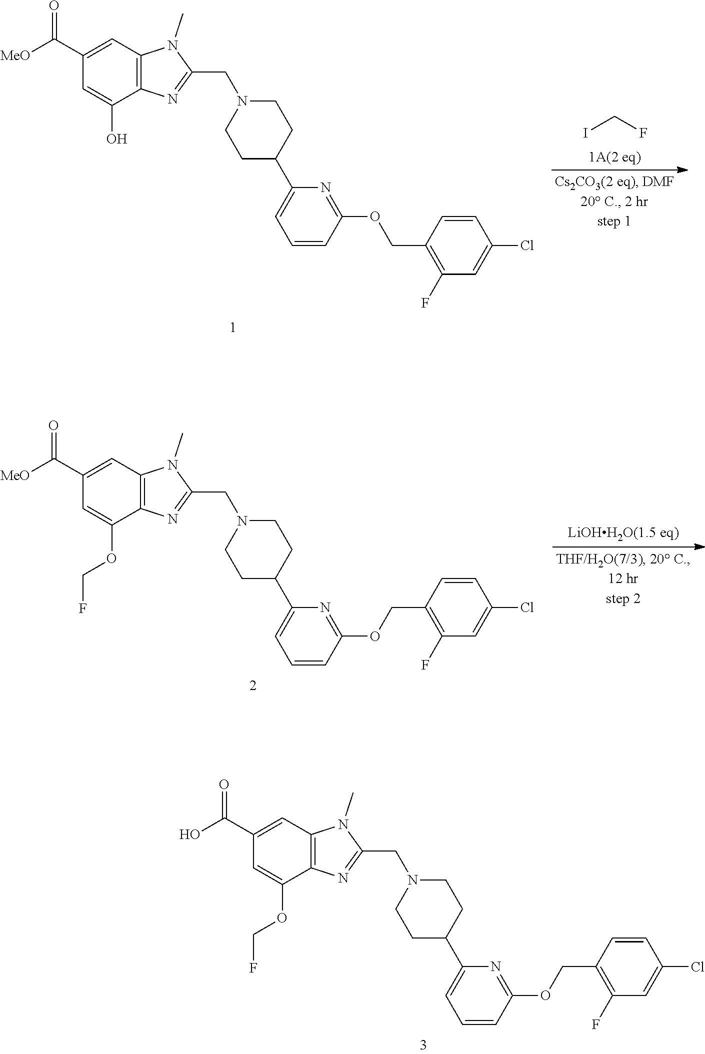

### General Procedure for Preparation of Intermediate 2:

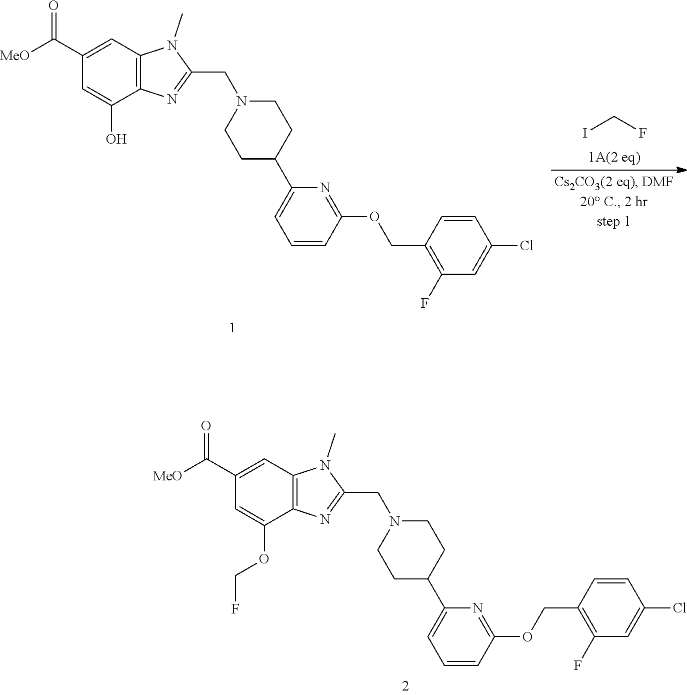

To a solution of 1 (60 mg, 111.32 μmol, 1 eq) and 1A (35.61 mg, 222.64 μmol, 2 eq) in DMF (1.5 mL) was added Cs₂CO₃ (72.54 mg, 222.64 μmol, 2 eq). The mixture was stirred at 20° C. for 2 hr under N₂ atmosphere. LCMS showed 1 was consumed completely and one main peak with desired mass was detected. The reaction mixture was quenched by addition water 5 mL, and then extracted with EA (5 mL*2). The combined organic layers were washed with NaCl (10 mL*2), dried over Na₂SO₄, filtered, and concentrated under reduced pressure to give a residue. The crude product was not purified and used to next step directly. 2 (70 mg, 148.42 μmol, 82.5% yield) was obtained as a yellow solid.

¹H NMR (400 MHz, DMSO-d₆) δ=8.00 (s, 1H), 7.62 (t, J=7.6 Hz, 1H), 7.55 (t, J=8.0 Hz, 1H), 7.49 (s, 1H), 7.45 (dd, J=2.0, 1.6 Hz, 1H), 7.28 (dd, J=1.6, 1.2 Hz, 1H), 6.86 (d, J=7.2 Hz, 1H), 6.66 (d, J=8.0 Hz, 1H), 6.13 (d, J=54.0 Hz, 2H), 5.35 (s, 2H), 3.96 (s, 3H), 3.89 (s, 3H), 3.84 (s, 2H), 2.93 (br d, J=11.2 Hz, 2H), 2.62-2.54 (m, 1H), 2.26-2.16 (m, 2H), 1.81-1.64 (m, 4H)

### General Procedure for Preparation of Compound 225 (3):

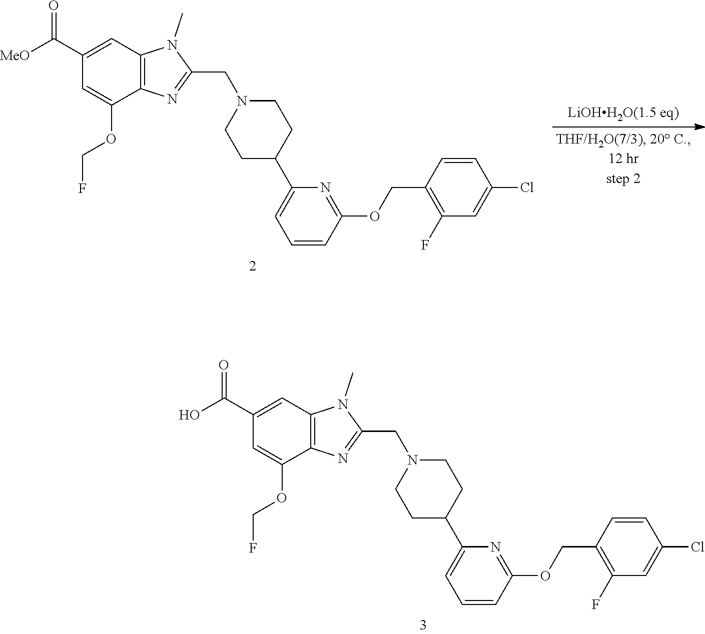 [SMILES: COC(=O)c1cc(OCF)c2nc(CN3CCC(c4cccc(OCc5ccc(Cl)cc5F)n4)CC3)n(C)c2c1 ; Cn1c(CN2CCC(c3cccc(OCc4ccc(Cl)cc4F)n3)CC2)nc2c(OCF)cc(C(=O)O)cc21]

To a solution of 2 (60 mg, 105.08 μmol, 1 eq) in THF (1.4 mL) was added LiOH·H₂O (6.61 mg, 157.61 μmol, 1.5 eq) in H₂O (0.6 mL). The mixture was stirred at 20° C. for 12 hr. LCMS showed 2 was consumed completely and one main peak with desired mass was detected. The crude reaction mixture on notebook page ET78548-85 (60 mg) was combined to ET78548-82 (10 mg) for workup. The mixture was filtered, and filtrate was concentrated in vacuum to get a residue. The residue was purified by prep-HPLC (column: 3_Phenomenex Luna C18 75*30 mm*3 um; mobile phase: [H₂O (0.2% FA)-ACN]; gradient: 30%-60% B over 8.0 min). 2-((4-(6-((4-Chloro-2-fluorobenzyl)oxy)pyridin-2-yl)piperidin-1-yl)methyl)-4-(fluoromethoxy)-1-methyl-1H-benzo[d]imidazole-6-carboxylic acid (23.52 mg, 42.23 μmol, 34.45% yield) was obtained as a white solid.

LCMS: RT=2.397 min, MS cal.: 556.2, [M+1]⁺=557.1

HPLC: purity: 97.9%

¹H NMR (400 MHz, DMSO-d₆) δ=7.94 (s, 1H), 7.62 (t, J=7.6 Hz, 1H), 7.55 (t, J=8.4 Hz, 1H), 7.49 (s, 1H), 7.45 (dd, J=2.0, 2.0 Hz, 1H), 7.28 (dd, J=2.0, 2.0 Hz, 1H), 6.86 (d, J=7.2 Hz, 1H), 6.66 (d, J=8.0 Hz, 1H), 6.11 (d, J=54.0 Hz, 2H), 5.36 (s, 2H), 3.93 (s, 3H), 3.83 (s, 2H), 2.93 (br d, J=11.2 Hz, 2H), 2.63-2.55 (m, 1H), 2.20 (br t, J=10.4 Hz, 2H), 1.83-1.64 (m, 4H)

### Example A146—Preparation of Compound 226. 2-((4-(6-((4-Chloro-2-methoxybenzyl)oxy)pyridin-2-yl)piperidin-1-yl)methyl)-4-(difluoromethoxy)-1-methyl-1H-benzo[d]imidazole-6-carboxylic acid (Compound 226) (4)

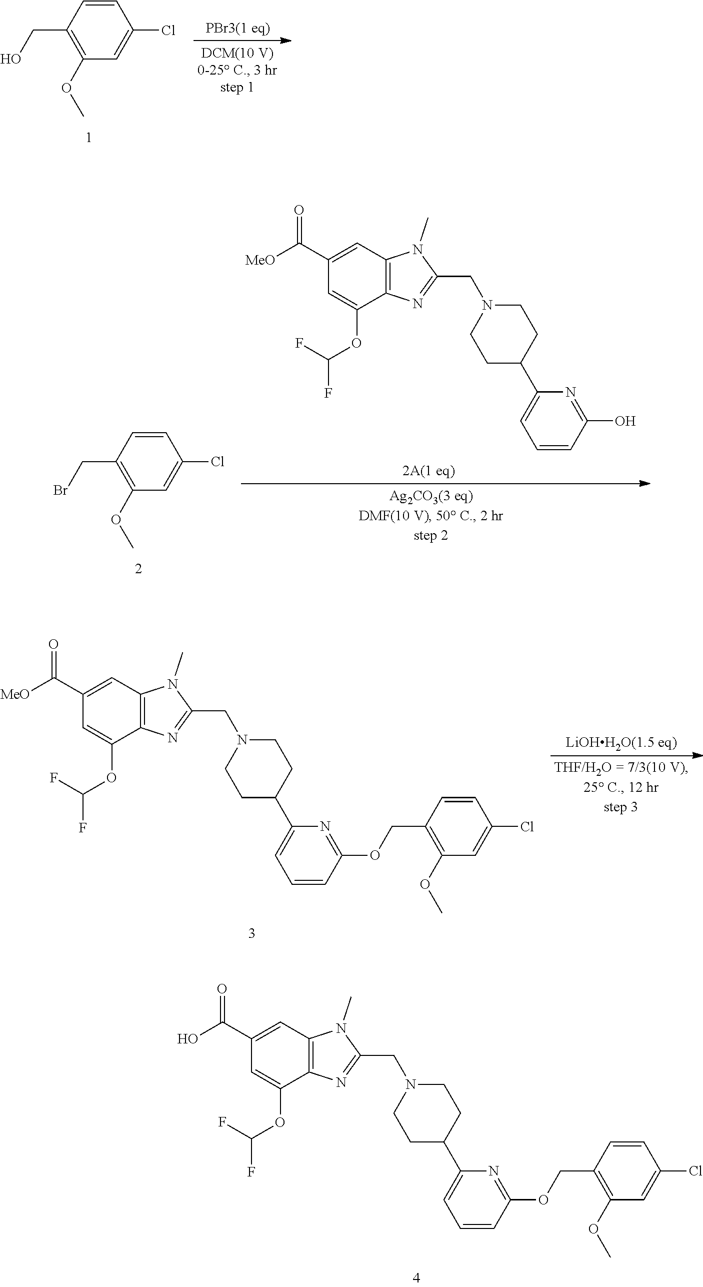 [SMILES: COc1cc(Cl)ccc1CO ; COc1cc(Cl)ccc1CBr ; COC(=O)c1cc(OC(F)F)c2nc(CN3CCC(c4cccc(O)n4)CC3)n(C)c2c1 ; COC(=O)c1cc(OC(F)F)c2nc(CN3CCC(c4cccc(OCc5ccc(Cl)cc5OC)n4)CC3)n(C)c2c1 ; COc1cc(Cl)ccc1COc1cccc(C2CCN(Cc3nc4c(OC(F)F)cc(C(=O)O)cc4n3C)CC2)n1]

### General Procedure for Preparation of Intermediate 2:

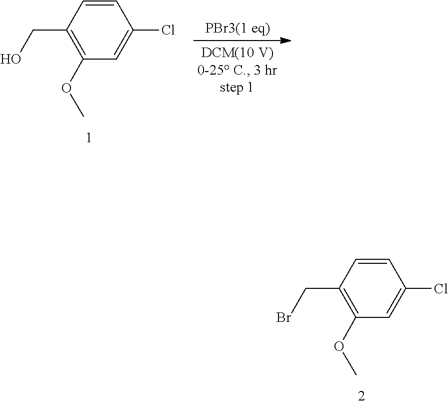

DCM (1 mL) was charged to the one-necked round bottom flask, then 1 (0.1 g, 579.35 μmol, 1 eq) was added to the mixture at 0° C. At 0° C., PBr₃ (156.82 mg, 579.35 μmol, 1 eq) was added to the reaction mixture at 0° C. After the addition, the mixture was stirred at 25° C. for 3 hr. LC-MS showed 1 was consumed completely and one main peak with desired was detected. After 3 hr, the reaction mixture was added to 10 mL H₂O at 25° C. The mixture was extracted by DCM (10 mL*3). Then organic phase was combined and washed by NaHCO₃ aqueous (10 mL*2), dried with Na₂SO₄, filtered. The organic phase was concentrated under reduced pressure at 45° C. to give a residue. 2 (0.12 g, 509.54 μmol, 87% yield) was obtained as a light yellow solid.

¹H NMR (400 MHz, CHCl3-d) δ=7.29 (s, 1H), 6.97-6.93 (m, 1H), 6.91-6.88 (m, 1H), 4.54 (s, 2H), 3.92 (s, 3H)

### General Procedure for Preparation of Intermediate 3:

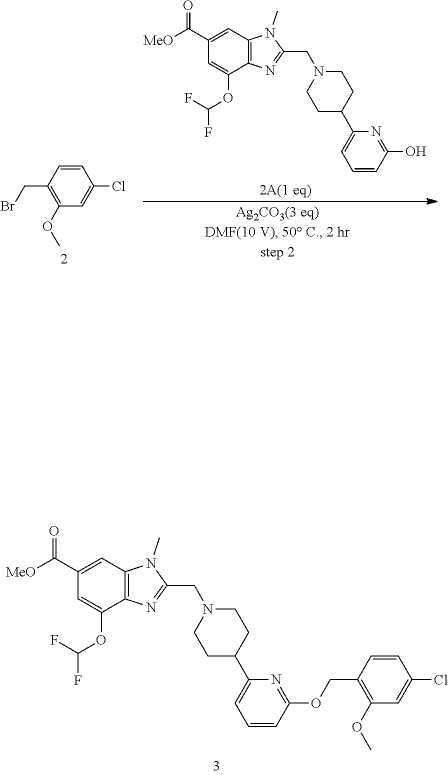 [SMILES: COc1cc(Cl)ccc1CBr ; COC(=O)c1cc(OC(F)F)c2nc(CN3CCC(c4cccc(O)n4)CC3)n(C)c2c1 ; COC(=O)c1cc(OC(F)F)c2nc(CN3CCC(c4cccc(OCc5ccc(Cl)cc5OC)n4)CC3)n(C)c2c1]

DMF (1.3 mL) was charged to the one-necked round bottom flask, then 2A (0.13 g, 291.19 μmol, 1 eq) and Ag₂CO₃ (240.88 mg, 873.57 μmol, 39.64 μL, 3 eq) was added to the mixture at 25° C. At 25° C., 2 (102.86 mg, 436.78 μmol, 1.5 eq) was added to the reaction mixture at 25° C. After the addition, the mixture was stirred at 50° C. for 2 hr. LC-MS showed 2A was consumed completely and one main peak with desired was detected. After 2 hr, the reaction mixture was added to 10 mL H₂O at 25° C. The mixture was extracted by DCM (10 mL*3). Then organic phase was combined and washed by NaHCO₃ aqueous (10 mL*2), dried with Na₂SO₄, filtered. The organic phase was concentrated under reduced pressure at 45° C. to give a residue. The residue was purified by prep-TLC (Petroleum ether:Ethyl acetate=1:1). 3 (0.1 g, 166.38 μmol, 57% yield) was obtained as a yellow solid.

LCMS: RT=0.523 min, MS cal.: 601,623, [M+H]⁺=600.20, 601.20, [M+23]⁺=623.3

¹H NMR (400 MHz, DMSO-d₆) δ=8.11 (d, J=1.2 Hz, 1H), 7.65 (t, J=74.4 Hz, 1H), 7.61 (t, J=8 Hz, 1H), 7.54-7.50 (m, 1H), 7.41-7.32 (m, 2H), 7.12-7.08 (m, 1H), 7.03-6.94 (m, 2H), 6.86 (s, 1H), 6.65 (d, J=8.0 Hz, 1H), 5.23-5.21 (m, 1H), 5.33-5.19 (m, 2H), 5.08 (t, J=5.6 Hz, 1H), 4.44 (d, J=5.6 Hz, 1H), 3.97 (s, 3H), 3.90 (s, 3H), 3.86 (s, 2H), 3.84 (s, 3H), 3.79 (s, 2H), 2.99-2.90 (m, 2H), 2.62-2.57 (m, 1H), 2.28-2.17 (m, 2H), 1.85-1.62 (m, 4H)

### General Procedure for Preparation of Compound 226 (4):

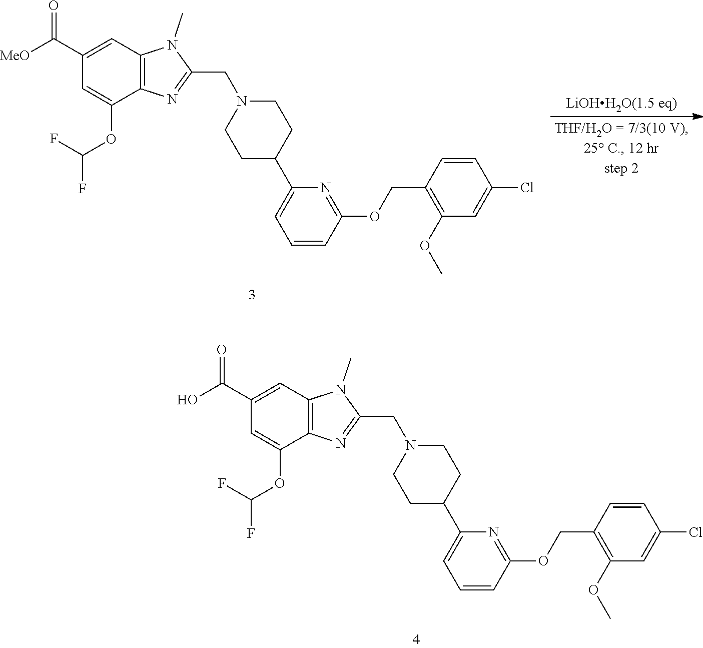 [SMILES: COC(=O)c1cc(OC(F)F)c2nc(CN3CCC(c4cccc(OCc5ccc(Cl)cc5OC)n4)CC3)n(C)c2c1 ; COc1cc(Cl)ccc1COc1cccc(C2CCN(Cc3nc4c(OC(F)F)cc(C(=O)O)cc4n3C)CC2)n1]

To a solution of 3 (85 mg, 141.42 μmol, 1 eq) in THF (0.63 mL) was added LiOH·H₂O (11.87 mg, 282.84 μmol, 2 eq) and H₂O (0.27 mL). The mixture was stirred at 25° C. for 12 hr. The mixture was monitoring by LCMS showed 2 was consumed completely and one main peak with desired m/z was detected. The residue was purified by prep-HPLC (column: Waters Xbridge Prep OBD C18 150*40 mm*10 um; mobile phase: [H₂O (10 mM NH₄HCO₃)-ACN]; gradient: 20%-60% B over 8.0 min). 2-((4-(6-((4-Chloro-2-methoxybenzyl)oxy)pyridin-2-yl)piperidin-1-yl)methyl)-4-(difluoromethoxy)-1-methyl-1H-benzo[d]imidazole-6-carboxylic acid (28.49 mg, 48.05 μmol, 33% yield, 99% purity) was obtained as a white solid.

LCMS: RT=2.802 min, MS cal.: 586.18, 588.18, [M+H]⁺=587.2

HPLC: RT=11.979 min, purity: 99.30%

¹H NMR (400 MHz, DMSO-d6) δ=8.07 (s, 1H), 7.64 (t, J=74.4 Hz, 1H), 7.61 (t, J=8.0 Hz, 1H), 7.54-7.51 (m, 1H), 7.39 (d, J=8.0 Hz, 1H), 7.11 (d, J=2.0 Hz, 1H), 6.99 (dd, J=2.0, 8.0 Hz, 1H), 6.85 (d, J=7.2 Hz, 1H), 6.66 (d, J=8.0 Hz, 1H), 5.28 (s, 2H), 3.96 (s, 3H), 3.85 (br s, 2H), 3.84 (s, 3H), 2.95 (br d, J=11.2 Hz, 2H), 2.62-2.55 (m, 1H), 2.23 (br t, J=10.8 Hz, 2H), 1.84-1.65 (m, 4H)

### Example A147—Preparation of Compound 227. 2-((4-(6-((4-Chloro-2-fluorobenzyl)oxy)pyridin-2-yl)piperidin-1-yl)methyl)-4-(1,1-difluoro-2-methoxyethoxy)-1-methyl-1H-benzo[d]imidazole-6-carboxylic acid (Compound 227) (8)

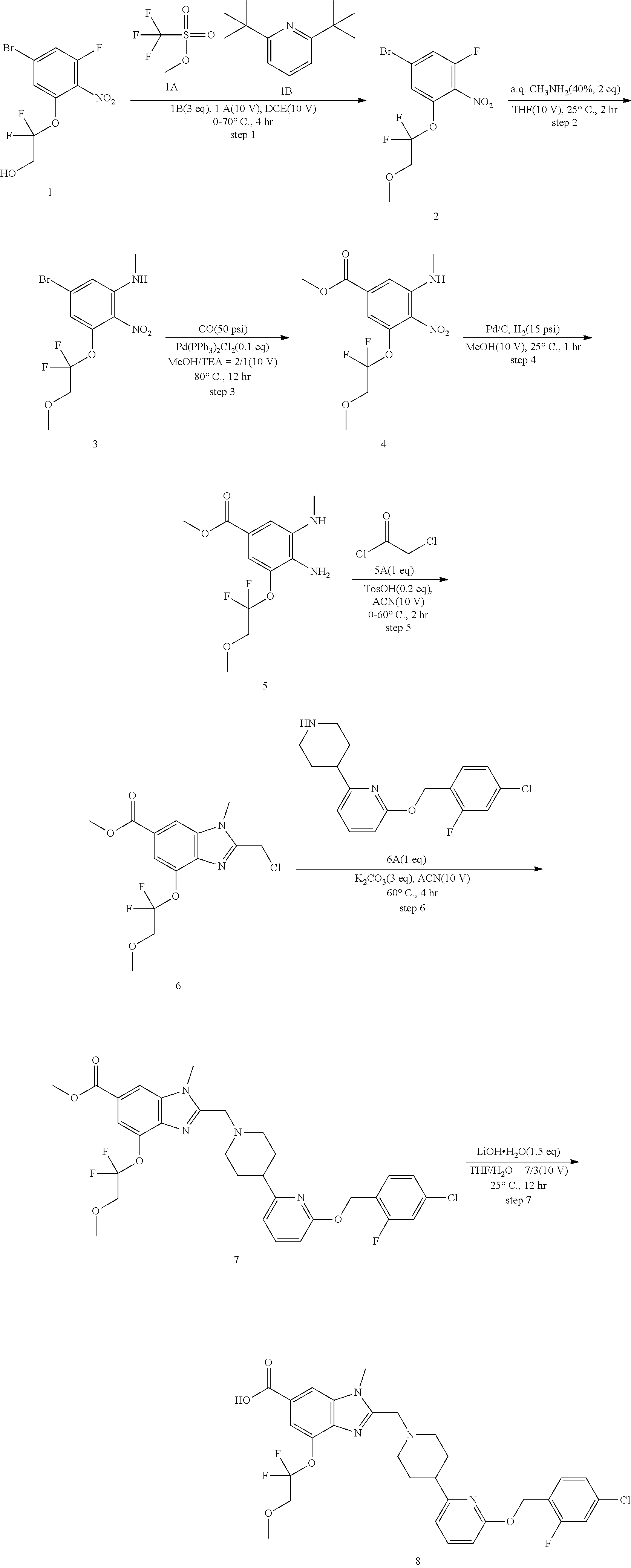

### General Procedure for Preparation of Intermediate 2:

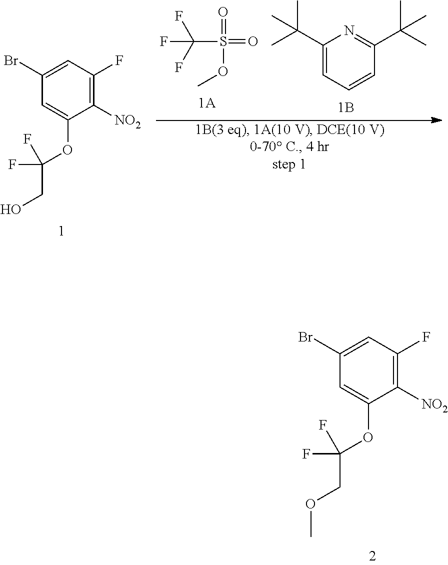

A mixture of 1 (500 mg, 1.58 mmol, 1 eq), 1B (908.05 mg, 4.75 mmol, 3 eq) in DCE (5 mL) was degassed and purged with N₂ for 3 times, and cooled to 0° C. 1A (7.48 g, 45.58 mmol, 5 mL, 28.81 eq) was added to the reaction mixture at 0° C. and then the mixture was stirred at 70° C. for 4 hrs under N₂ atmosphere. TLC indicated 1 was consumed completely and two new spots formed. The reaction was clean according to TLC (Petroleum ether:Ethyl acetate=5:1, Product Rr-0.53). The residue mixture was diluted with H₂O 30 mL and extracted with DCM (30 mL*3). The combined organic layers were washed with brine 30 mL, dried over Na₂SO₄, filtered and concentrated under reduced pressure to give a residue. The residue was purified by column chromatography (SiO₂, Petroleum ether/Ethyl acetate=100/1 to 40/1, TLC-Petroleum ether/Ethyl acetate=5/1, Product R_{f}=0.53). 2 (510 mg, 1.45 mmol, 91.88% yield) was obtained as a colorless liquid.

LCMS: RT=1.304 min, MS cal.: 328.95, [M+H]⁺=279.9

¹H NMR (400 MHz, CHCl3-d) δ=7.50 (d, J=1.2 Hz, 1H), 7.36 (dd, J=1.6, 8.5 Hz, 1H), 3.85 (t, J=9.6 Hz, 2H), 3.53 (s, 3H)

### General Procedure for Preparation of Intermediate 3:

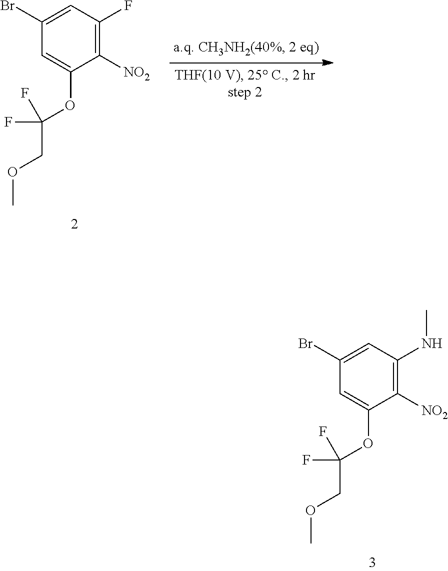 [SMILES: COCC(F)(F)Oc1cc(Br)cc(F)c1[N+](=O)[O-] ; CNc1cc(Br)cc(OC(F)(F)COC)c1[N+](=O)[O-]]

A mixture of 2 (500 mg, 1.51 mmol, 1 eq) in THF (5 mL) was degassed and purged with N₂ for 3 times. a.q. CH₃NH₂ (235.24 mg, 3.03 mmol, 40% purity, 2 eq) was added to the reaction mixture at 25° C. and then the mixture was stirred at 25° C. for 2 hrs under N₂ atmosphere. TLC indicated 2 was consumed completely and one new spot formed. The reaction was clean according to TLC (Petroleum ether:Ethyl acetate=5:1, Product R_{f}=0.41). The reaction mixture was adjusted to pH=6-7 with FA (1 M) and diluted with H₂O 30 mL. The mixture was extracted with EtOAc (30 mL*3). The combined organic layers were washed with brine 10 mL, dried over Na₂SO₄, filtered and concentrated under reduced pressure to give a residue. 3 (500 mg, 1.37 mmol, 90.58% yield) was obtained as a yellow solid.

LCMS: RT=0.549 min, MS cal.: 339.99, [M+H]⁺=412.1

¹H NMR (400 MHz, CHLOROFORM-d) δ=6.90-6.82 (m, 2H), 6.47 (br s, 1H), 3.87 (t, J=9.6 Hz, 2H), 3.55 (s, 3H), 2.95 (d, J=4.4 Hz, 3H)

### General Procedure for Preparation of Intermediate 4:

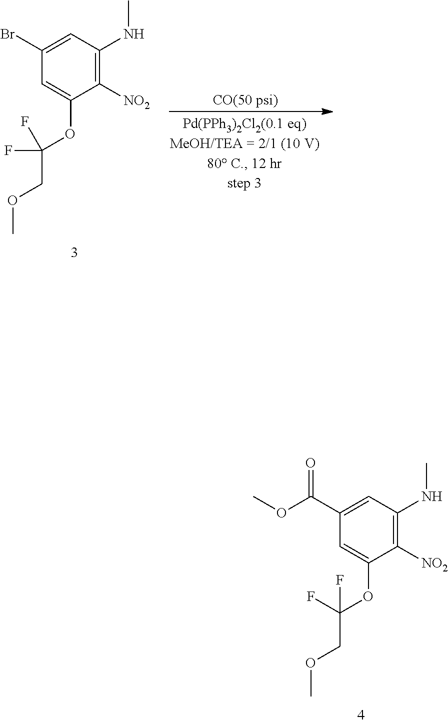 [SMILES: CNc1cc(Br)cc(OC(F)(F)COC)c1[N+](=O)[O-] ; CNc1cc(C(=O)OC)cc(OC(F)(F)COC)c1[N+](=O)[O-]]

A mixture of 3 (500 mg, 1.47 mmol, 1 eq) and Pd(PPh₃)₂Cl₂ (102.89 mg, 146.58 μmol, 0.1 eq) in MeOH (4 mL) and TEA (2 mL) was degassed and purged with CO for 3 times, and then the mixture was stirred at 80° C. for 12 hr under CO atmosphere (50 psi). TLC indicated 3 was consumed completely and one new spot formed. The reaction was clean according to TLC (Petroleum ether:Ethyl acetate=5:1, Product R_{f}=0.31). The residue mixture was diluted with H₂O 30 mL and extracted with EtOAc (30 mL*3). The combined organic layers were washed with brine 30 mL, dried over Na₂SO₄, filtered and concentrated under reduced pressure to give a residue. The residue was purified by column chromatography (SiO₂, Petroleum ether/Ethyl acetate=100/1 to 5/1, TLC-Petroleum ether/Ethyl acetate=5/1, Product R_{f}=0.31). 4 (340 mg, 1.02 mmol, 69.25% yield) was obtained as a white solid.

LCMS: RT=1.260 min, MS cal.: 320.08, [M+H]⁺=320.9

¹H NMR (400 MHz, CHLOROFORM-d) δ=7.38 (d, J=1.2 Hz, 1H), 7.32 (d, J=1.2 Hz, 1H), 6.22-6.01 (m, 1H), 3.96 (s, 3H), 3.87 (t, J=9.6 Hz, 2H), 3.56 (s, 3H), 3.00 (s, 3H)

### General Procedure for Preparation of Intermediate 5:

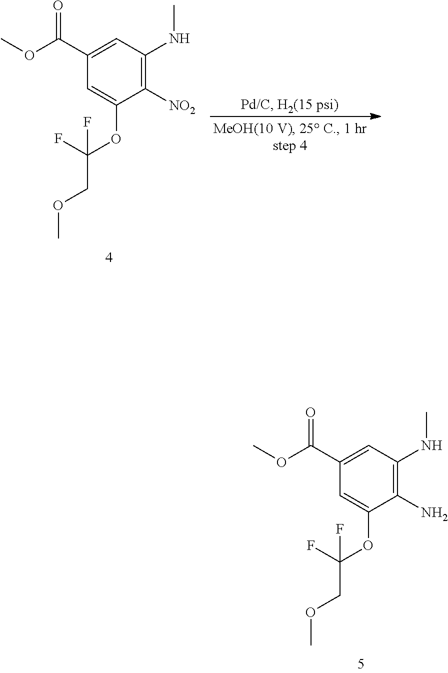

A mixture of 4 (300 mg, 936.78 μmol, 1 eq) in MeOH (3 mL) was degassed and purged with N₂ for 3 times. Pd/C (150.00 mg) was added to the reaction mixture at 25° C. and the suspension was degassed and purged with H₂ for 3 times. The mixture was stirred under H₂ (15 Psi) at 25° C. for 1 hr. LC-MS showed 4 was consumed completely and one main peak with desired mass was detected. The reaction mixture was filtered, and filtrate was concentrated under reduced pressure to give a residue. 5 (260 mg, 815.12 μmol, 87.01% yield) was obtained as a white solid.

LCMS: RT=0.644 min, MS cal.: 290.11, [M+H]⁺=291.2

¹H NMR (400 MHz, DMSO-d6) δ=7.13 (d, J=1.2 Hz, 1H), 6.86 (d, J=1.6 Hz, 1H), 5.26 (s, 2H), 5.18-5.10 (m, 1H), 4.00 (t, J=10.0 Hz, 2H), 3.77-3.74 (m, 3H), 3.45 (s, 3H), 2.76 (d, J=4.8 Hz, 3H)

### General Procedure for Preparation of Intermediate 6:

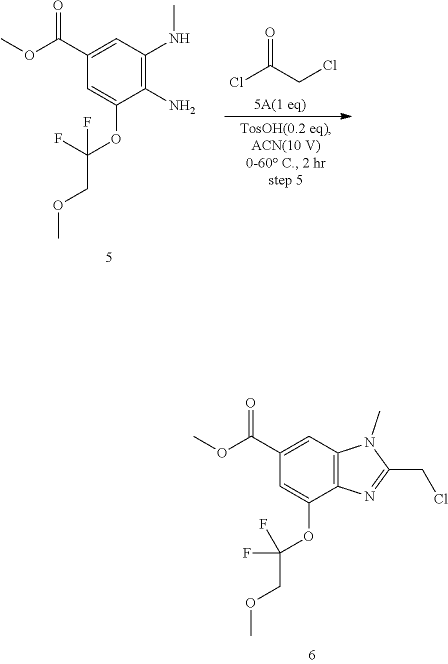

A mixture of 5 (260 mg, 895.74 μmol, 1 eq), TosOH (30.85 mg, 179.15 μmol, 0.2 eq) in ACN (2.1 mL) was degassed and purged with N₂ for 3 times, and cooled to 0° C. 5A (101.17 mg, 895.74 μmol, 71.35 μL, 1 eq) in ACN (0.5 mL) was added to the reaction mixture at 0° C. and then the mixture was stirred at 60° C. for 2 hrs under N₂ atmosphere. LC-MS showed 5 was consumed completely and one main peak with desired mass was detected. The reaction mixture was concentrated under reduced pressure to remove solvent. 6 (280 mg, crude) was obtained as a colorless oil.

LCMS: RT=0.449 min, MS cal.: 348.07, [M+H]⁺=349.0

### General Procedure for Preparation of Intermediate 7:

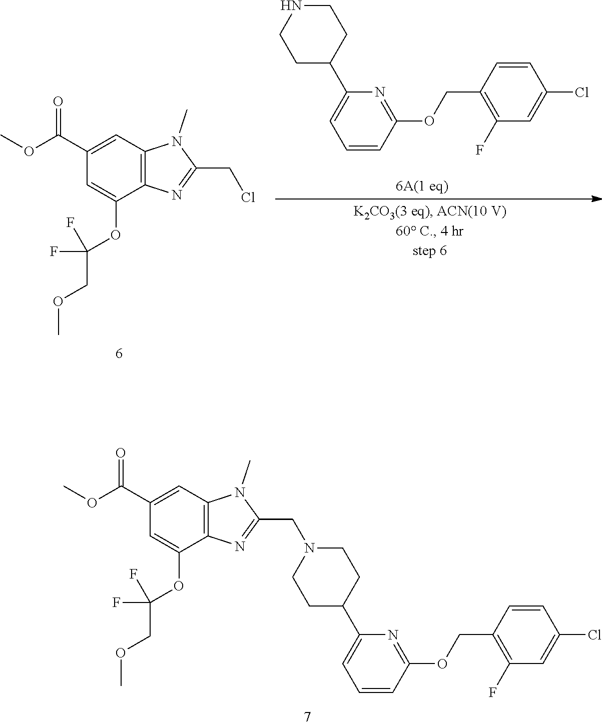 [SMILES: COCC(F)(F)Oc1cc(C(=O)OC)cc2c1nc(CCl)n2C ; Fc1cc(Cl)ccc1COc1cccc(C2CCNCC2)n1 ; COCC(F)(F)Oc1cc(C(=O)OC)cc2c1nc(CN1CCC(c3cccc(OCc4ccc(Cl)cc4F)n3)CC1)n2C]

A mixture of 6 (280 mg, 802.92 μmol, 1 eq), 6A (257.57 mg, 802.92 μmol, 1 eq) and K₂CO₃ (332.90 mg, 2.41 mmol, 3 eq) in ACN (2.8 mL) was degassed and purged with N₂ for 3 times and then the mixture was stirred at 60° C. for 4 hrs under N₂ atmosphere. LC-MS showed 6 was consumed completely and one main peak with desired mass was detected. The residue mixture was diluted with H₂O 30 mL and extracted with DCM (30 mL*3). The combined organic layers were washed with brine 30 mL, dried over Na₂SO₄, filtered, and concentrated under reduced pressure to give a residue. The residue was purified by column chromatography (SiO₂, Petroleum ether/Ethyl acetate=100/1 to 1/1, TLC-Petroleum ether/Ethyl acetate=1/1, Product R_{f}=0.44). 7 (260 mg, 392.76 μmol, 48.92% yield) was obtained as a white solid.

LCMS: RT=1.707 min, MS cal.: 632.20, [M+H]⁺=633.1

¹H NMR (400 MHz, CHLOROFORM-d) δ=8.03 (s, 1H), 7.91 (d, J=1.2 Hz, 1H), 7.50 (t, J=7.6 Hz, 1H), 7.44 (t, J=8.0 Hz, 1H), 7.15-7.08 (m, 2H), 6.74 (d, J=7.2 Hz, 1H), 6.61 (d, J=8.0 Hz, 1H), 5.41 (s, 2H), 4.08-3.98 (m, 6H), 3.97 (s, 3H), 3.92 (br s, 1H), 3.61 (s, 3H), 3.05-2.88 (m, 2H), 2.61 (br s, 1H), 2.39-2.20 (m, 2H), 1.96-1.78 (m, 4H)

### General Procedure for Preparation of Compound 227 (8):

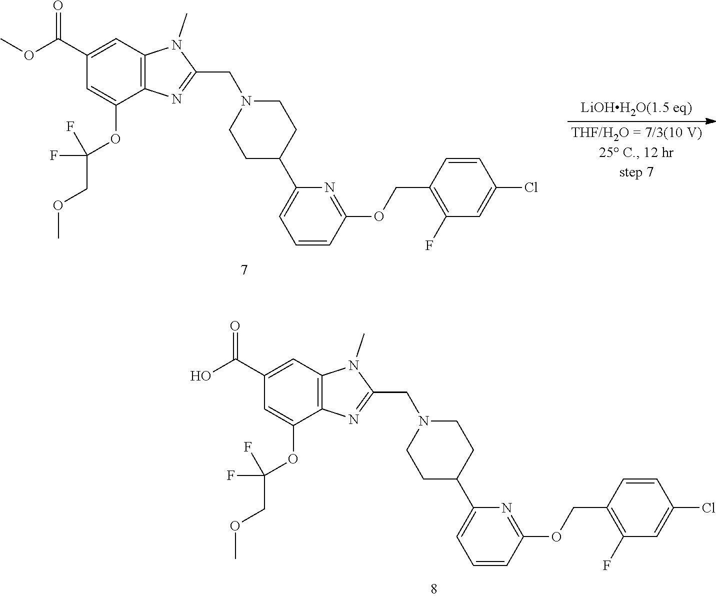 [SMILES: COCC(F)(F)Oc1cc(C(=O)OC)cc2c1nc(CN1CCC(c3cccc(OCc4ccc(Cl)cc4F)n3)CC1)n2C ; COCC(F)(F)Oc1cc(C(=O)O)cc2c1nc(CN1CCC(c3cccc(OCc4ccc(Cl)cc4F)n3)CC1)n2C]

A mixture of 7 (100 mg, 157.96 μmol, 1 eq), LiOH·H₂O (9.94 mg, 236.95 μmol, 1.5 eq) in THF (0.7 mL) and H₂O (0.3 mL) was degassed and purged with N₂ for 3 times, and then the mixture was stirred at 25° C. for 12 hrs under N₂ atmosphere. LC-MS (ET62904-669-p1a1) showed ˜48% of 7 (Rt=0.499 min) remained. Several new peaks were shown on LC-MS and ˜45% of desired Compound (Rt=0.463) was detected. The reaction mixture was diluted with ACN 0.5 mL. The liquid was purified by prep-HPLC (column: Waters Xbridge Prep OBD C₁₈ 150*40 mm*10 um; mobile phase: [H₂O (10 mM NH₄HCO₃)-ACN]; gradient: 35%-85% B over 8.0 min). 2-((4-(6-((4-Chloro-2-fluorobenzyl)oxy)pyridin-2-yl)piperidin-1-yl)methyl)-4-(1,1-difluoro-2-methoxyethoxy)-1-methyl-1H-benzo[d]imidazole-6-carboxylic acid (29.15 mg, 47.09 μmol, 29.81% yield, 100% purity) was obtained as a white solid.

LCMS: RT=0. 0.463 min, MS cal.: 618.19, [M+H]⁺=619.2

¹H NMR (400 MHz, METHANOL-d₄) δ=8.14 (d, J=1.2 Hz, 1H), 7.85 (d, J=1.2 Hz, 1H), 7.57 (t, J=7.6 Hz, 1H), 7.48 (t, J=8.0 Hz, 1H), 7.23-7.13 (m, 2H), 6.82 (d, J=7.2 Hz, 1H), 6.63 (d, J=8.0 Hz, 1H), 5.40 (s, 2H), 4.07-4.01 (m, 5H), 3.97 (s, 2H), 3.56 (s, 3H), 3.07 (br d, J=11.2 Hz, 2H), 2.71-2.61 (m, 1H), 2.44-2.32 (m, 2H), 1.92-1.84 (m, 4H)

### Example A148—Preparation of Compound 228. 4-(Difluoromethoxy)-2-((4-(6-((2-fluoro-4-methoxybenzyl)oxy)pyridin-2-yl)piperidin-1-yl)methyl)-1-methyl-1H-benzo[d]imidazole-6-carboxylic acid (Compound 228) (3)

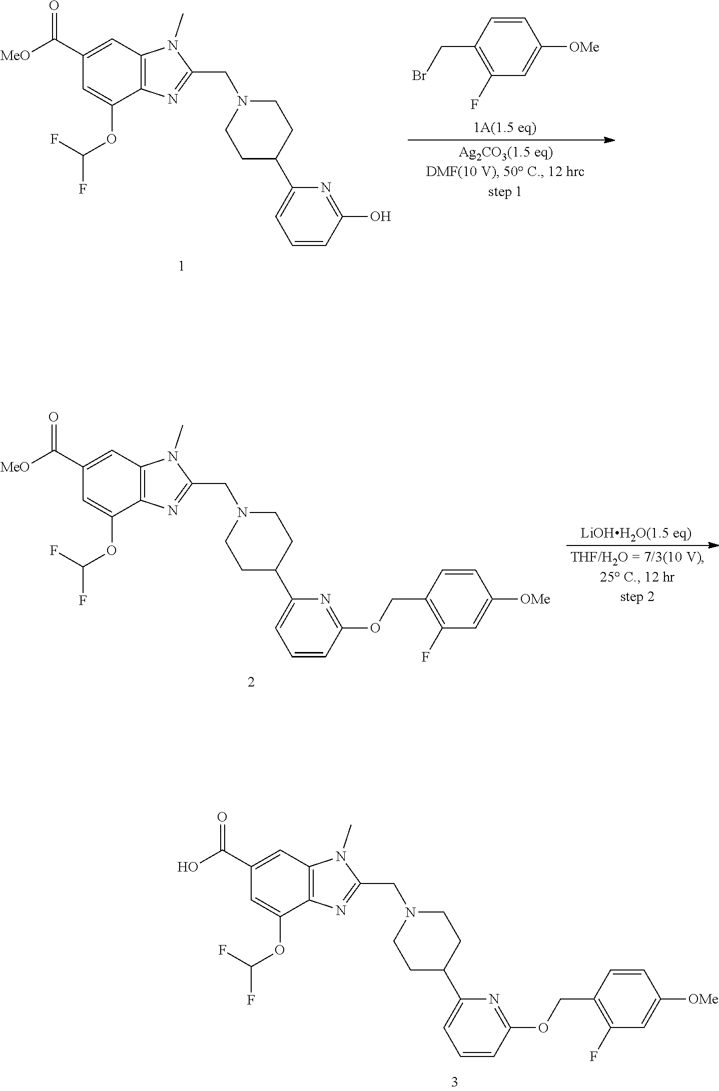 [SMILES: COC(=O)c1cc(OC(F)F)c2nc(CN3CCC(c4cccc(O)n4)CC3)n(C)c2c1 ; COc1ccc(CBr)c(F)c1 ; COC(=O)c1cc(OC(F)F)c2nc(CN3CCC(c4cccc(OCc5ccc(OC)cc5F)n4)CC3)n(C)c2c1 ; COc1ccc(COc2cccc(C3CCN(Cc4nc5c(OC(F)F)cc(C(=O)O)cc5n4C)CC3)n2)c(F)c1]

### General Procedure for Preparation of Intermediate 2:

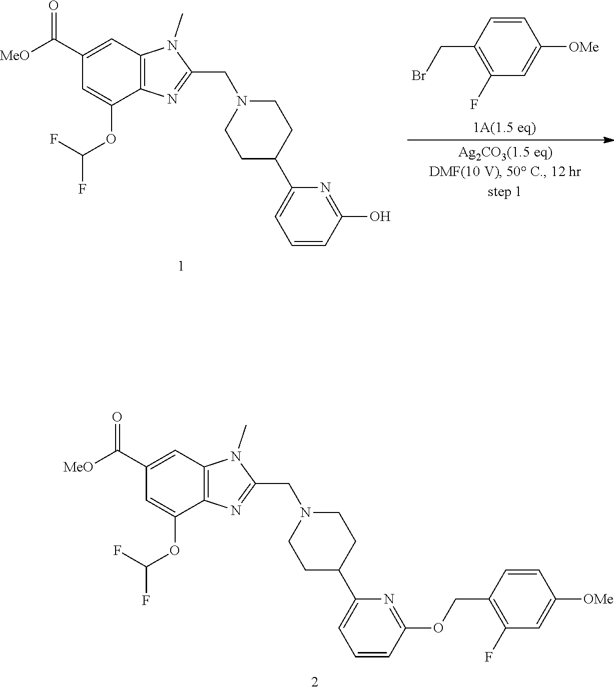 [SMILES: COC(=O)c1cc(OC(F)F)c2nc(CN3CCC(c4cccc(O)n4)CC3)n(C)c2c1 ; COc1ccc(CBr)c(F)c1 ; COC(=O)c1cc(OC(F)F)c2nc(CN3CCC(c4cccc(OCc5ccc(OC)cc5F)n4)CC3)n(C)c2c1]

To a solution of 1 (100 mg, 223.99 μmol, 1 eq) and 1A (73.60 mg, 335.99 μmol, 1.5 eq) in DMF (1 mL) was added Ag₂CO₃ (92.65 mg, 335.99 μmol, 15.25 μL, 1.5 eq). The mixture was stirred at 50° C. for 12 hr. The mixture was monitoring by LCMS showed 1 was consumed completely and one main peak with desired m/z was detected. TLC (Petroleum ether:Ethyl acetate=1:1) indicated 1 was consumed completely and one new spot formed. The reaction was clean according to TLC. The reaction mixture was added to H₂O 10 mL at 20° C. The mixture was extracted by DCM (10 mL*3). Then organic phase was combined and washed by brine (10 mL), dried with Na₂SO₄, filtered. The organic phase was concentrated under reduced pressure at 30° C. to give a residue. The residue was purified by column chromatography (SiO₂, Petroleum ether/Ethyl acetate=100/0 to 95/5). 2 (130 mg, 193.47 μmol, 86.37% yield, 87% purity) was obtained as a green oil.

LCMS: RT=1.600 min, MS cal.: 584.22, 585.23, [M+H]⁺=585.1

¹H NMR (400 MHz, DMSO-d₆) δ=8.12 (d, J=1.2 Hz, 1H), 7.66 (t, J=74.4 Hz, 1H), 7.60 (t, J=8.0 Hz, 1H), 7.54 (s, 1H), 7.47-7.42 (m, 1H), 6.88-6.81 (m, 2H), 6.76 (dd, J=2.4, 8.4 Hz, 1H), 6.62 (d, J=8.0 Hz, 1H), 5.29 (s, 2H), 3.99 (s, 3H), 3.90 (s, 3H), 3.87 (s, 2H), 3.75 (s, 3H), 2.96 (br d, J=11.2 Hz, 2H), 2.65-2.55 (m, 1H), 2.24 (br t, J=10.8 Hz, 2H), 1.87-1.78 (m, 3H), 1.78-1.69 (m, 2H)

### General Procedure for Preparation of Compound 228 (3):

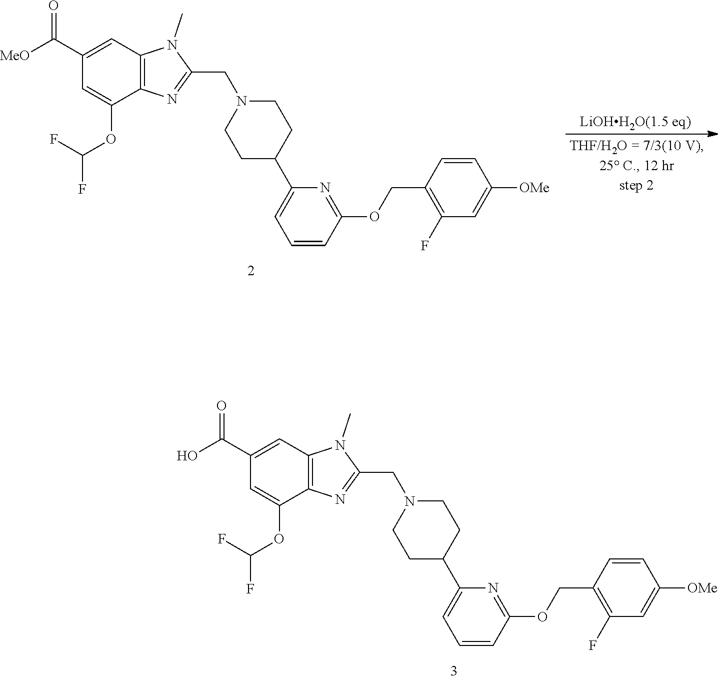 [SMILES: COC(=O)c1cc(OC(F)F)c2nc(CN3CCC(c4cccc(OCc5ccc(OC)cc5F)n4)CC3)n(C)c2c1 ; COc1ccc(COc2cccc(C3CCN(Cc4nc5c(OC(F)F)cc(C(=O)O)cc5n4C)CC3)n2)c(F)c1]

To a solution of 1 (100 mg, 171.06 μmol, 1 eq) in THF (0.7 mL) and H₂O (0.3 mL) was added LiOH·H₂O (10.77 mg, 256.59 μmol, 1.5 eq). The mixture was stirred at 25° C. for 12 hr. The reaction was monitored by LCMS showed 2 was consumed completely and one main peak with desired m/z was detected. The residue was purified by prep-HPLC (column: Waters Xbridge Prep OBD C18 150*40 mm*10 um; mobile phase: [H₂O (10 mM NH₄HCO₃)-ACN]; gradient: 25%-75% B over 8.0 min). 4-(Difluoromethoxy)-2-((4-(6-((2-fluoro-4-methoxybenzyl)oxy)pyridin-2-yl)piperidin-1-yl)methyl)-1-methyl-1H-benzo[d]imidazole-6-carboxylic acid (29.04 mg, 50.13 μmol, 29.31% yield, 98.5% purity) was obtained as a white solid.

LCMS: RT=2.661 min, MS cal.: 570.21, 571.21, [M+H]⁺=571.2

HPLC: RT=11.061 min, purity: 98.51%

¹H NMR (400 MHz, DMSO-d₆) δ=8.06 (s, 1H), 7.63 (t, J=74.4 Hz, 1H), 7.60 (t, J=7.8 Hz, 1H), 7.52 (s, 1H), 7.48-7.42 (m, 1H), 6.86-6.80 (m, 2H), 6.75 (dd, J=2.4, 8.4 Hz, 1H), 6.61 (d, J=8.2 Hz, 1H), 5.28 (s, 2H), 3.97 (s, 3H), 3.86 (s, 2H), 3.74 (s, 3H), 2.95 (br d, J=11.2 Hz, 2H), 2.65-2.55 (m, 1H), 2.23 (br t, J=10.4 Hz, 2H), 1.85-1.68 (m, 4H)

### Example A149—Preparation of Compound 229. 4-(Difluoromethoxy)-1-methyl-2-((4-(6-((5-(trifluoromethyl)pyridin-2-yl)methoxy)pyridin-2-yl)piperidin-1-yl)methyl)-1H-benzo[d]imidazole-6-carboxylic acid (Compound 229) (3)

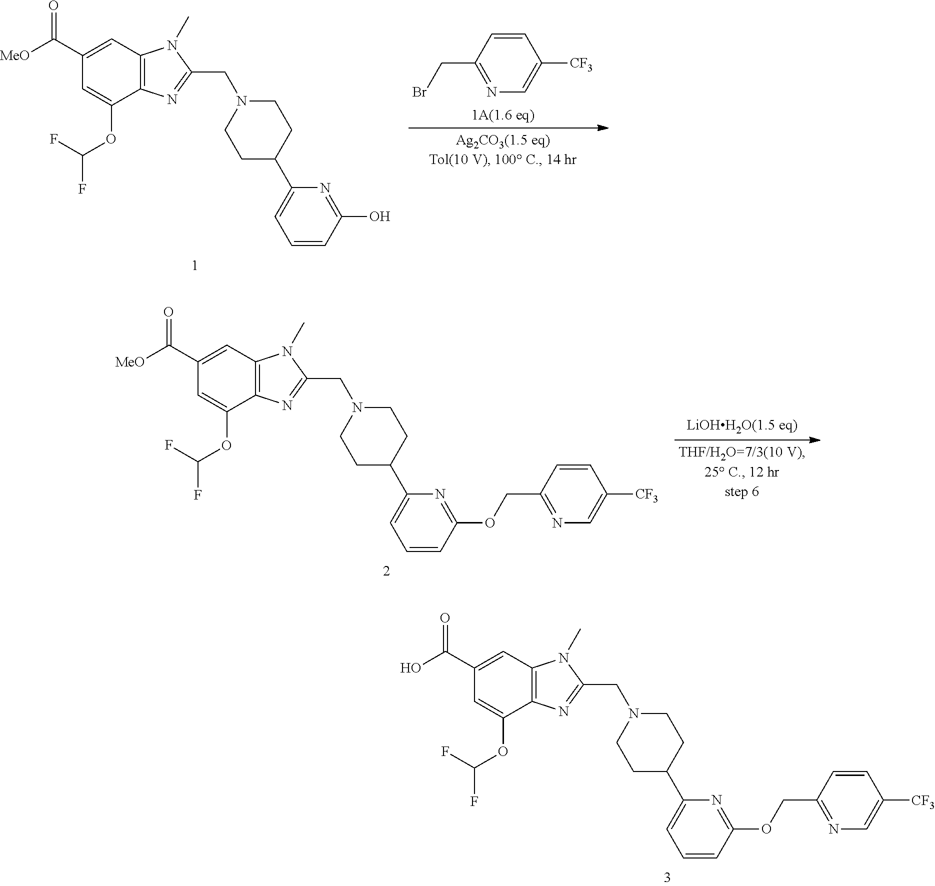 [SMILES: COC(=O)c1cc(OC(F)F)c2nc(CN3CCC(c4cccc(O)n4)CC3)n(C)c2c1 ; FC(F)(F)c1ccc(CBr)nc1 ; COC(=O)c1cc(OC(F)F)c2nc(CN3CCC(c4cccc(OCc5ccc(C(F)(F)F)cn5)n4)CC3)n(C)c2c1 ; Cn1c(CN2CCC(c3cccc(OCc4ccc(C(F)(F)F)cn4)n3)CC2)nc2c(OC(F)F)cc(C(=O)O)cc21]

### General Procedure for Preparation of Intermediate 2:

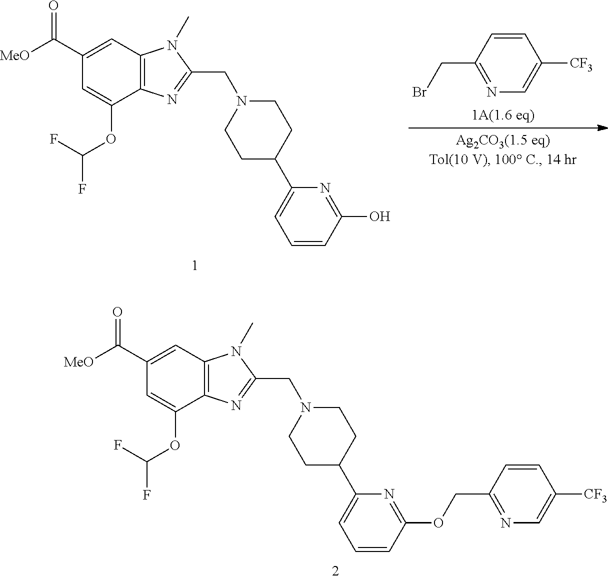 [SMILES: COC(=O)c1cc(OC(F)F)c2nc(CN3CCC(c4cccc(O)n4)CC3)n(C)c2c1 ; FC(F)(F)c1ccc(CBr)nc1 ; COC(=O)c1cc(OC(F)F)c2nc(CN3CCC(c4cccc(OCc5ccc(C(F)(F)F)cn5)n4)CC3)n(C)c2c1]

To a solution of 1 (130 mg, 291.19 μmol, 1 eq) in toluene (2 mL) was added Ag₂CO₃ (120.44 mg, 436.78 μmol, 19.82 L, 1.5 eq) and 1A (76.88 mg, 320.31 μmol, 1.1 eq). The mixture was stirred at 100° C. for 12 hr. After monitoring, the reaction was not completely. The mixture was added Compound 1A (34.95 mg, 145.59 μmol, 0.5 eq). The mixture was stirred at 100° C. for 2 hr. LCMS showed 1 was consumed completely and one main peak with desired mass was detected. The reaction mixture was filtered and the filter cake was washed with EtOAc (20 mL). The reaction mixture was diluted with H₂O 10 mL and extracted with EtOAc 20 mL (10 mL*2). The combined organic layers were washed with brine 10 mL, dried over Na₂SO₄, filtered and concentrated under reduced pressure to give a residue. The residue was purified by column chromatography (SiO₂, PE/EtOAc=1/0 to 1/1). 2 (80 mg, 114.94 μmol, 39.47% total yield) was obtained as a brown oil.

LCMS: RT=1.599 min, MS cal.: 605.2/606.2, [M+H]⁺=606.2/607.2

¹HNMR (400 MHz, CHCl3-d) δ=8.85 (s, 1H), 8.00 (s, 1H), 7.94-7.89 (m, 1H), 7.72 (s, 1H), 7.62-7.51 (m, 2H), 7.50-7.47 (m, 1H), 7.30 (br s, 1H), 7.11 (br s, 1H), 6.74 (br dd, J=8.0, 15.6 Hz, 2H), 5.58 (s, 2H), 4.03-3.96 (m, 6H), 3.93-3.77 (m, 2H), 3.05-2.78 (m, 2H), 2.71-2.52 (m, 1H), 2.41-2.11 (m, 2H), 1.87-1.77 (m, 2H), 1.63-1.53 (m, 2H)

### General Procedure for Preparation of Compound 229 (3):

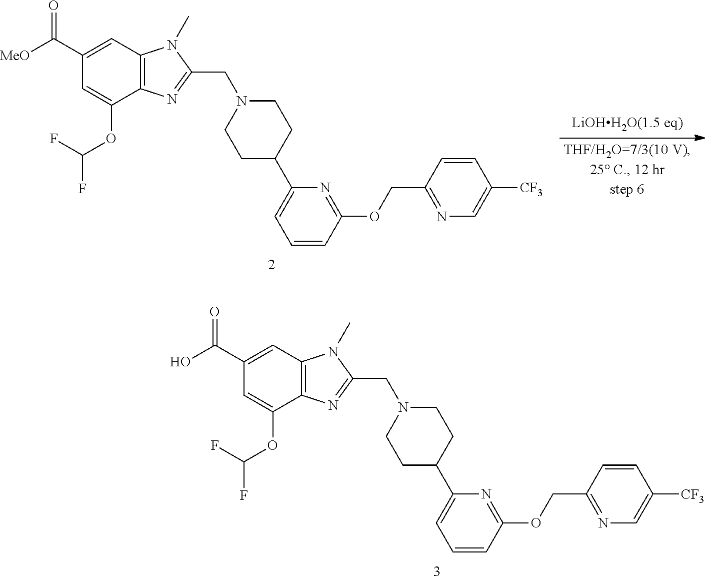 [SMILES: COC(=O)c1cc(OC(F)F)c2nc(CN3CCC(c4cccc(OCc5ccc(C(F)(F)F)cn5)n4)CC3)n(C)c2c1 ; Cn1c(CN2CCC(c3cccc(OCc4ccc(C(F)(F)F)cn4)n3)CC2)nc2c(OC(F)F)cc(C(=O)O)cc21]

To a solution of 2 (70 mg, 115.60 μmol, 1 eq) in THF (0.7 mL) and H₂O (0.3 mL) was added LiOH·H₂O (7.28 mg, 173.39 μmol, 1.5 eq). The mixture was stirred at 25° C. for 12 hr. LCMS showed 2 was consumed completely and one main peak with desired mass was detected. The mixture was added ACN 0.1 mL. The residue was purified by prep-HPLC (neutral condition: column: Waters Xbridge Prep OBD C18 150*40 mm*10 um; mobile phase: [H₂O (10 mM NH₄HCO₃)-ACN]; gradient: 10%-60% B over 8.0 min). 4-(Difluoromethoxy)-1-methyl-2-((4-(6-((5-(trifluoromethyl)pyridin-2-yl)methoxy)pyridin-2-yl)piperidin-1-yl)methyl)-1H-benzo[d]imidazole-6-carboxylic acid (26.44 mg, 43.80 μmol, 37.89% total yield) was obtained as a white solid.

LCMS: RT=2.604 min, MS cal.: 591.2/592.2, [M+H]⁺=592.2/593.2

HPLC: RT=11.069 min

¹HNMR (400 MHz, MeOH-d4) δ=8.82 (s, 1H), 8.12 (s, 1H), 8.10-8.05 (m, 1H), 7.68 (s, 1H), 7.65 (d, J=8.4 Hz, 1H), 7.61 (dd, J=7.6, 8.2 Hz, 1H), 7.44-7.05 (m, 1H), 6.83 (d, J=7.6 Hz, 1H), 6.77-6.73 (m, 1H), 5.55 (s, 2H), 4.00 (s, 3H), 3.99-3.92 (m, 2H), 3.03 (br dd, J=3.2, 5.2 Hz, 2H), 2.66-2.56 (m, 1H), 2.45-2.29 (m, 2H), 1.82-1.67 (m, 4H)

### Example A150—Preparation of Compound 230. 2-((4-(3-((4-Chloro-2-fluorobenzyl)oxy)-4-methoxyphenyl)piperidin-1-yl)methyl)-4-(difluoromethoxy)-1-methyl-1H-benzo[d]imidazole-6-carboxylic acid (Compound 230) (7)

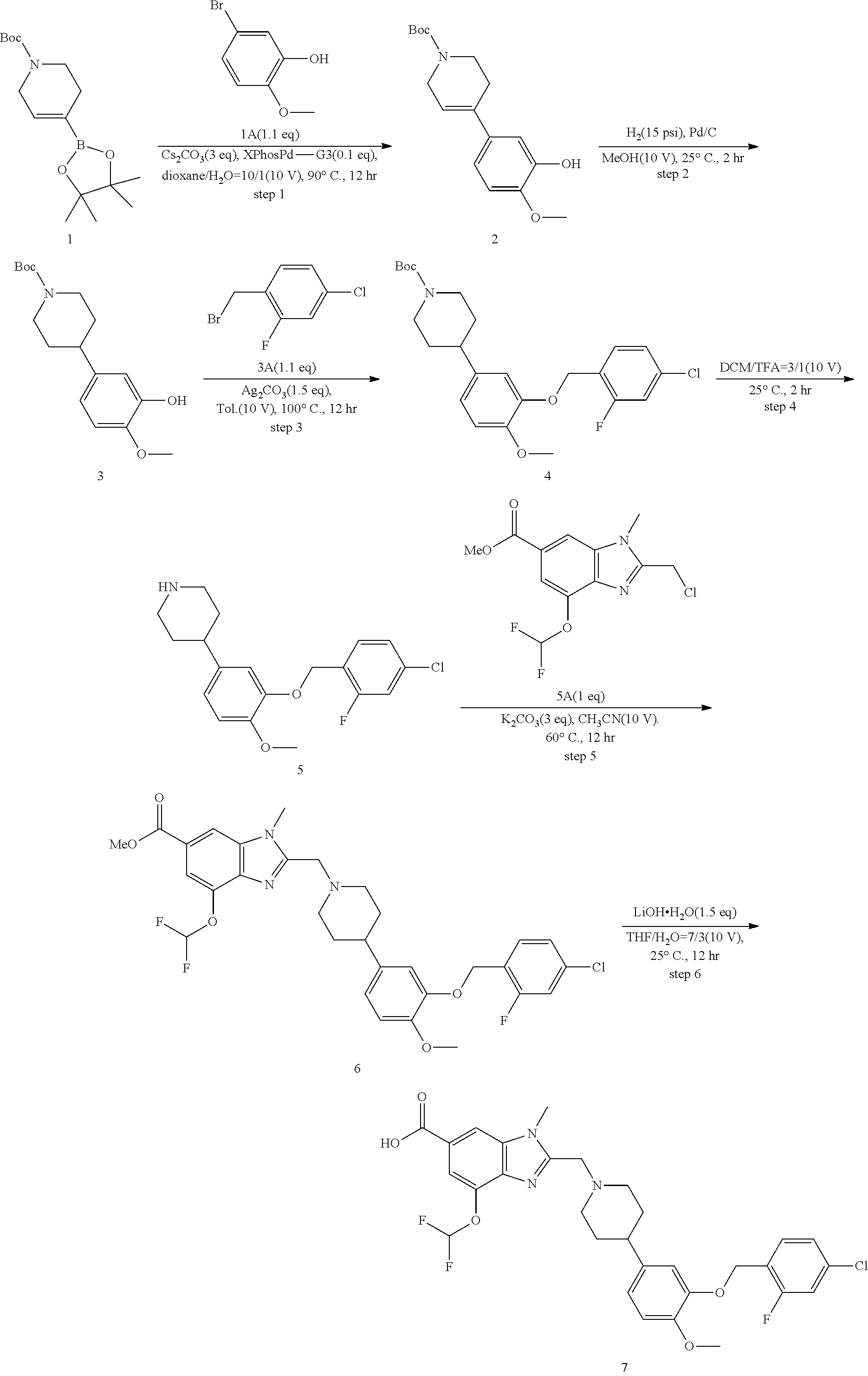

### General Procedure for Preparation of Intermediate 2:

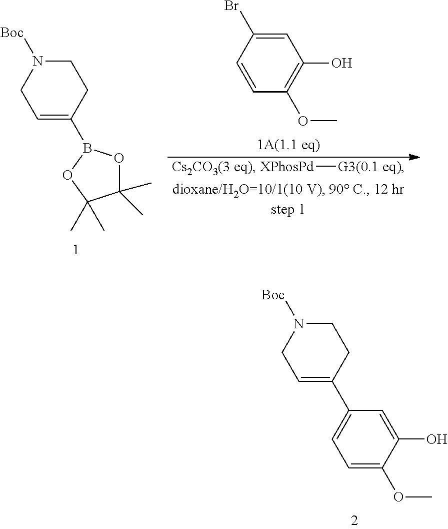

To a solution of 1 (1 g, 3.23 mmol, 1 eq) in dioxane (10 mL) and H₂O (1 mL) was added Cs₂CO₃ (3.16 g, 9.70 mmol, 3 eq), 1A (722.29 mg, 3.56 mmol, 1.1 eq) and XPhos Pd G3 (273.75 mg, 323.41 μmol, 0.1 eq). The mixture was stirred at 90° C. for 12 hr. LCMS showed 1 was consumed completely and one main peak with desired mass was detected. The reaction mixture was filtered and the filter cake was washed with EtOAc 20 mL, the filtrates was diluted with H₂O 10 mL and extracted with EtOAc 90 mL (30 mL*3). The combined organic layers were washed with brine 20 mL, dried over Na₂SO₄, filtered and concentrated under reduced pressure to give a residue. The residue was purified by column chromatography (SiO₂, PE/EtOAc=1/0 to 5/1). 2 (0.5 g, 1.56 mmol, 48.10% yield) was obtained as a white solid.

LCMS: RT=0.780 min, MS cal.: 305.2/306.2, [M−55]⁺=250.1/251.1

¹HNMR (400 MHz, CHCl3-d) δ=6.99 (d, J=2.0 Hz, 1H), 6.90-6.86 (m, 1H), 6.83-6.80 (m, 1H), 5.95 (br s, 1H), 5.62 (s, 1H), 4.06 (br d, J=2.4 Hz, 2H), 3.90 (s, 3H), 3.62 (t, J=5.6 Hz, 2H), 2.48 (br s, 2H), 1.50 (s, 9H)

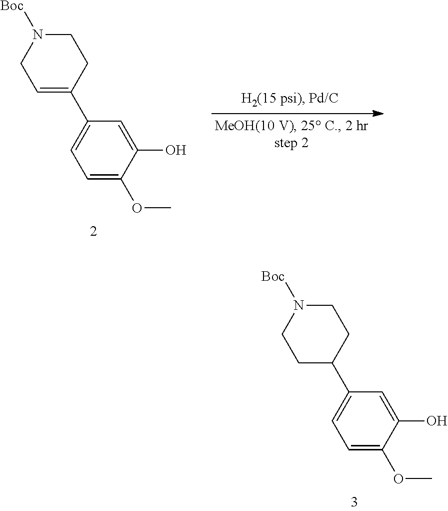 [SMILES: COc1ccc(C2=CCN(C(=O)OC(C)(C)C)CC2)cc1O ; COc1ccc(C2CCN(C(=O)OC(C)(C)C)CC2)cc1O]

Equip a 25 mL one-necked round bottom flask, thermometer, H₂ balloon. MeOH (4 mL) was charged to the 25 mL one-necked round bottom flask, then 2 (400 mg, 1.31 mmol, 1 eq) was added to the mixture at 25° C. Pd/C (199.34 mg) was added dropwise to the reaction mixture at 25° C. After the addition, the mixture was stirred at 25° C. for 2 hr under H₂ (15 psi). LCMS showed 2 was consumed completely and one main peak with desired mass was detected. The reaction mixture was filtered, and the filter cake was washed with MeOH. The combined filtrates were concentrated to dryness to give product. 3 (440 mg, crude) was obtained as a white solid.

LCMS: RT=0.806 min, MS cal.: 307.18/308.18, [M−55]⁺=252.1/253.1

¹HNMR (400 MHz, CHCl3-d) δ=6.82-6.78 (m, 2H), 6.69 (dd, J=2.0, 8.4 Hz, 1H), 5.58 (br s, 1H), 4.23 (br d, J=13.2 Hz, 2H), 3.88 (s, 3H), 2.78 (dt, J=2.4, 12.8 Hz, 2H), 2.60-2.51 (m, 1H), 1.80 (br d, J=13.2 Hz, 2H), 1.59 (br d, J=4.0 Hz, 2H), 1.49 (s, 9H)

### General Procedure for Preparation of Intermediate 4:

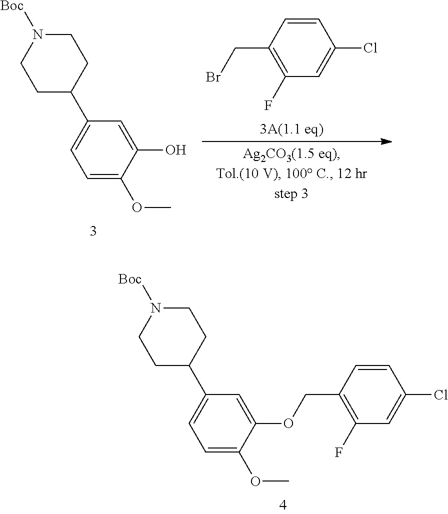 [SMILES: COc1ccc(C2CCN(C(=O)OC(C)(C)C)CC2)cc1O ; Fc1cc(Cl)ccc1CBr ; COc1ccc(C2CCN(C(=O)OC(C)(C)C)CC2)cc1OCc1ccc(Cl)cc1F]

To a solution of 3 (430 mg, 1.40 mmol, 1 eq) in Toluene (4.5 mL) was added Ag₂CO₃ (578.61 mg, 2.10 mmol, 95.21 μL, 1.5 eq) and 3A (343.87 mg, 1.54 mmol, 1.1 eq). The mixture was stirred at 100° C. for 12 hr. LCMS showed 3 was consumed completely and one main peak with desired mass was detected. The reaction mixture was filtered, and the filter cake was washed with EtOAc (20 mL). The reaction mixture was diluted with H₂O 5 mL and extracted with EtOAc 20 mL (10 mL*2). The combined organic layers were washed with brine 10 mL, dried over Na₂SO₄, filtered and concentrated under reduced pressure to give a residue. The residue was purified by column chromatography (SiO₂, PE/EtOAc=1/0 to 9/1). 4 (270 mg, 492.06 μmol, 35.17% yield) was obtained as a colorless oil.

LCMS: RT=1.706 min, MS cal.: 449.18/451.17, [M−55]⁺=394.1/396.1

¹HNMR (400 MHz, CHCl3-d) δ=7.50 (t, J=8.0 Hz, 1H), 7.14 (ddd, J=2.0, 9.2, 14.8 Hz, 2H), 6.87-6.84 (m, 1H), 6.82-6.75 (m, 2H), 5.15 (s, 2H), 4.23 (br d, J=13.2 Hz, 2H), 3.87 (s, 3H), 2.78 (dt, J=2.0, 12.8 Hz, 2H), 2.60-2.51 (m, 1H), 1.78 (br d, J=13.2 Hz, 2H), 1.58-1.56 (m, 2H), 1.49 (s, 9H)

### General Procedure for Preparation of Intermediate 5:

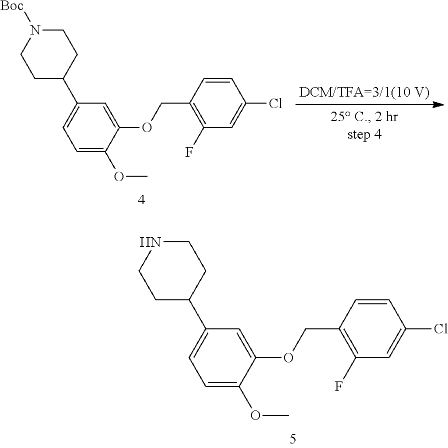 [SMILES: COc1ccc(C2CCN(C(=O)OC(C)(C)C)CC2)cc1OCc1ccc(Cl)cc1F ; COc1ccc(C2CCNCC2)cc1OCc1ccc(Cl)cc1F]

To a solution of 4 (100 mg, 222.25 μmol, 1 eq) in DCM (1 mL) was added TFA (460.50 mg, 4.04 mmol, 0.3 mL, 18.17 eq). The mixture was stirred at 25° C. for 2 hr. LCMS showed 4 was consumed completely and one main peak with desired mass was detected. The reaction mixture was concentrated under reduced pressure. 5 (100 mg, crude, TFA salt) was obtained as a colorless oil.

LCMS: RT=0.400 min, MS cal.: 349.1/351.1, [M+1]⁺=350.0/352.0

¹HNMR (400 MHz, CHCl3-d) δ=7.47 (t, J=8.0 Hz, 1H), 7.18-7.10 (m, 2H), 6.91-6.88 (m, 1H), 6.84-6.77 (m, 2H), 5.15 (s, 2H), 3.88 (s, 3H), 3.64 (br d, J=12.0 Hz, 2H), 3.19-3.05 (m, 2H), 2.82-2.69 (m, 1H), 2.09 (br d, J=13.6 Hz, 2H), 2.04-1.92 (m, 2H)

### General Procedure for Preparation of Intermediate 6:

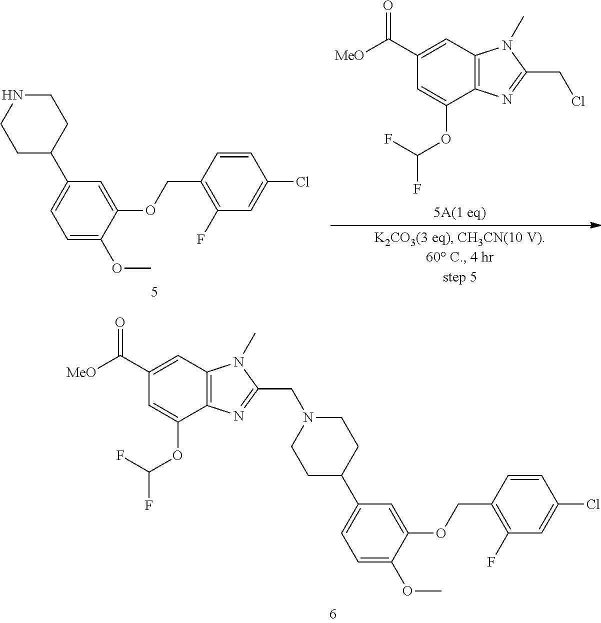 [SMILES: COc1ccc(C2CCNCC2)cc1OCc1ccc(Cl)cc1F ; COC(=O)c1cc(OC(F)F)c2nc(CCl)n(C)c2c1 ; COC(=O)c1cc(OC(F)F)c2nc(CN3CCC(c4ccc(OC)c(OCc5ccc(Cl)cc5F)c4)CC3)n(C)c2c1]

To a solution of 5 (100 mg, 215.59 μmol, 1 eq, TFA salt) in CH₃CN (1 mL) was added K₂CO₃ (89.39 mg, 646.76 μmol, 3 eq) and 5A (65.68 mg, 215.59 μmol, 1 eq). The mixture was stirred at 60° C. for 12 hr. LCMS showed 5 was consumed completely and one main peak with desired mass was detected. The reaction mixture was diluted with H₂O 5 mL and extracted with EtOAc 10 mL (5 mL*2). The combined organic layers were washed with brine 10 mL, dried over Na₂SO₄, filtered, and concentrated under reduced pressure to give a residue. The residue was purified by column chromatography (SiO₂, PE/EtOAc=1/0 to 1/1). 6 (60 mg, 78.64 μmol, 36.48% yield) was obtained as a brown oil.

LCMS: RT=1.638 min, MS cal.: 617.2/618.2, [M+1]⁺=618.2/619.2

### General Procedure for Preparation of Compound 230 (7):

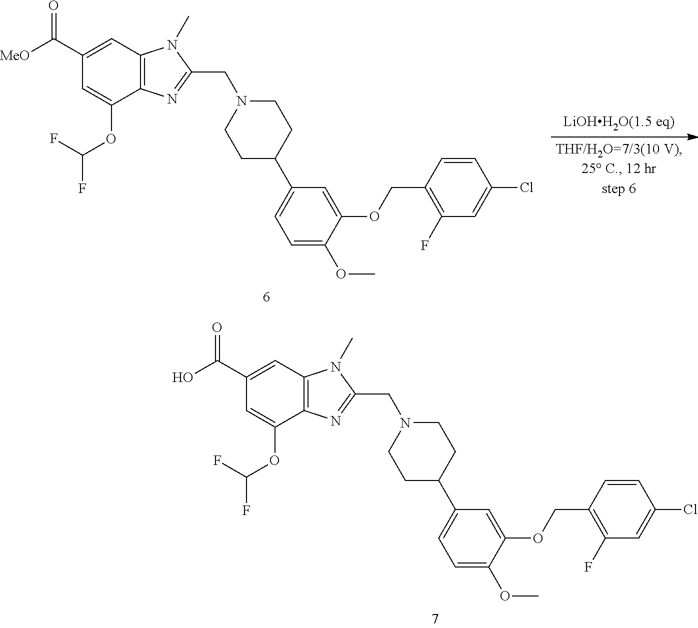 [SMILES: COC(=O)c1cc(OC(F)F)c2nc(CN3CCC(c4ccc(OC)c(OCc5ccc(Cl)cc5F)c4)CC3)n(C)c2c1 ; COc1ccc(C2CCN(Cc3nc4c(OC(F)F)cc(C(=O)O)cc4n3C)CC2)cc1OCc1ccc(Cl)cc1F]

To a solution of 6 (60 mg, 97.08 μmol, 1 eq) in THF (0.7 mL) and H₂O (0.3 mL) was added LiOH·H₂O (6.11 mg, 145.62 μmol, 1.5 eq). The mixture was stirred at 25° C. for 12 hr. LCMS showed 6 was consumed completely and one main peak with desired mass was detected. The mixture was added ACN 0.5 mL. The residue was purified by prep-HPLC (neutral condition: column: Waters Xbridge Prep OBD C18 150*40 mm*10 um; mobile phase: [H₂O (10 mM NH₄HCO₃)-ACN]; gradient: 20%-70% B over 8.0 min). 2-((4-(3-((4-Chloro-2-fluorobenzyl)oxy)-4-methoxyphenyl)piperidin-1-yl)methyl)-4-(difluoromethoxy)-1-methyl-1H-benzo[d]imidazole-6-carboxylic acid (17.66 mg, 29.09 μmol, 29.96% yield) was obtained as a white solid.

LCMS: RT=2.736 min, MS cal.: 604.2/606.2, [M+H]⁺=605.2.2/607.2

HPLC: RT=12.047 min

¹H NMR (400 MHz, MeOH-d4) δ=8.12-8.10 (m, 1H), 7.70-7.67 (m, 1H), 7.54-7.48 (m, 1H), 7.25 (t, J=74.4 Hz, 1H), 7.24-7.19 (m, 2H), 6.92-6.88 (m, 1H), 6.87-6.82 (m, 2H), 5.10 (s, 2H), 4.02 (s, 3H), 3.98-3.95 (m, 2H), 3.81-3.79 (m, 3H), 3.11-3.05 (m, 2H), 2.56-2.47 (m, 1H), 2.43-2.34 (m, 2H), 1.85-1.67 (m, 4H)

### Example A151—Preparation of Compound 231. 4-(Difluoromethoxy)-2-((4-(6-((4-fluoro-2-methoxybenzyl)oxy)pyridin-2-yl)piperidin-1-yl)methyl)-1-methyl-1H-benzo[d]imidazole-6-carboxylic acid (Compound 231) (3)

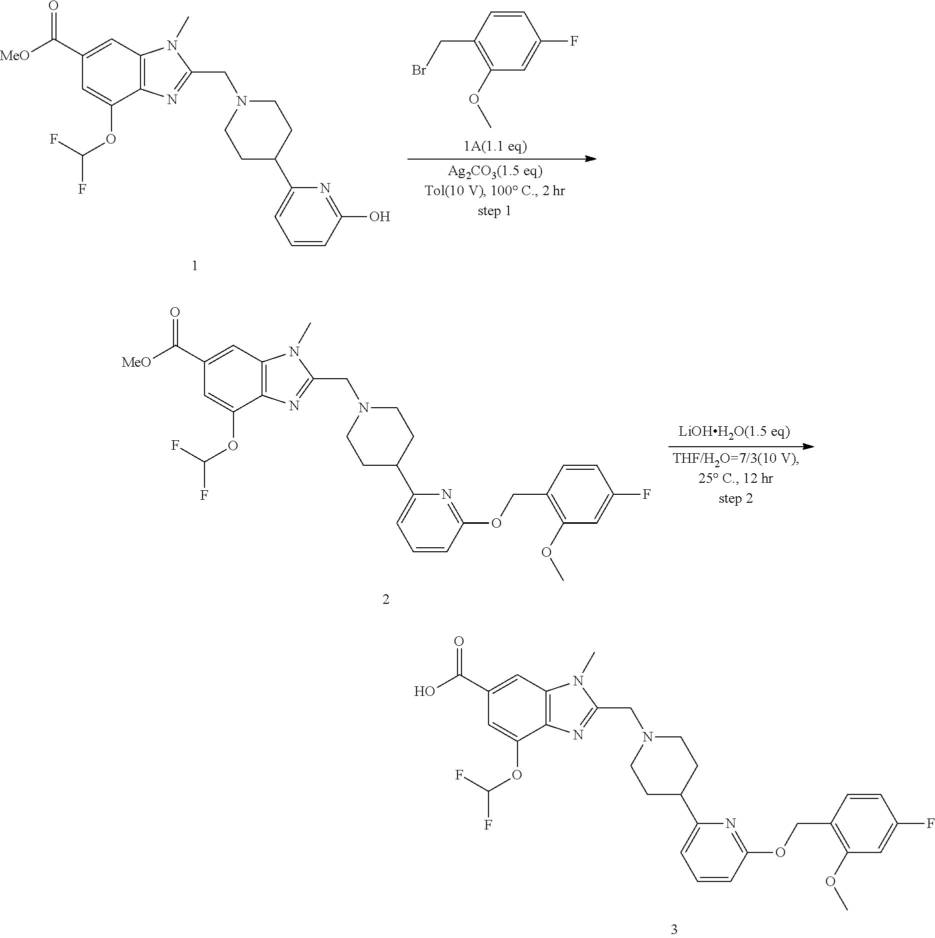 [SMILES: COC(=O)c1cc(OC(F)F)c2nc(CN3CCC(c4cccc(O)n4)CC3)n(C)c2c1 ; COc1cc(F)ccc1CBr ; COC(=O)c1cc(OC(F)F)c2nc(CN3CCC(c4cccc(OCc5ccc(F)cc5OC)n4)CC3)n(C)c2c1 ; COc1cc(F)ccc1COc1cccc(C2CCN(Cc3nc4c(OC(F)F)cc(C(=O)O)cc4n3C)CC2)n1]

### General Procedure for Preparation of Intermediate 2:

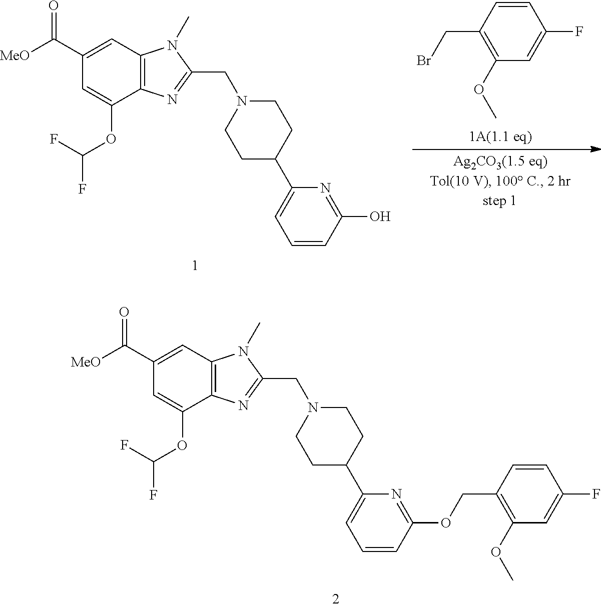 [SMILES: COC(=O)c1cc(OC(F)F)c2nc(CN3CCC(c4cccc(O)n4)CC3)n(C)c2c1 ; COc1cc(F)ccc1CBr ; COC(=O)c1cc(OC(F)F)c2nc(CN3CCC(c4cccc(OCc5ccc(F)cc5OC)n4)CC3)n(C)c2c1]

A mixture of 1 (200 mg, 447.98 μmol, 1 eq), 1A (107.94 mg, 492.78 μmol, 1.1 eq), Ag₂CO₃ (185.29 mg, 671.97 μmol, 30.49 μL, 1.5 eq) in toluene (2 mL) was degassed and purged with N₂ for 3 times, and then the mixture was stirred at 100° C. for 2 hr under N₂ atmosphere. TLC (Petroleum ether/Ethyl acetate=0:1, R_{f}=0.46) indicated ˜0% of 1 was remained, and one major new spot with lower polarity was detected. The mixture was filtered and the filtrate was diluted with H₂O 10 mL and extracted with EtOAc (10 mL*3). The combined organic layers were washed with brine (10 mL*2), dried over Na₂SO₄, filtered, and concentrated under reduced pressure to give a residue. The residue was purified by column chromatography (SiO₂, Petroleum ether/Ethyl acetate=100/1 to 0/1). 2 (170 mg, 290.80 μmol, 64.91% yield) was obtained as a white solid.

LCMS: RT=0.973 min, MS cal.: 584.2, [M−H]⁻=585.4

¹H NMR (400 MHz, CHCl3-d) δ=7.99 (s, 1H), 7.72 (s, 1H), 7.49 (t, J=7.6 Hz, 1H), 7.41 (br t, J=7.6 Hz, 1H), 7.31 (s, 1H), 6.72 (d, J=7.2 Hz, 1H), 6.67-6.59 (m, 3H), 5.37-5.31 (m, 2H), 4.01 (br s, 3H), 3.97 (s, 3H), 3.91 (br s, 1H), 3.85 (s, 3H), 3.06-2.91 (m, 2H), 2.72-2.58 (m, 1H), 2.42-2.23 (m, 2H), 2.01-1.79 (m, 4H)

### General Procedure for Preparation of Compound 231 (3):

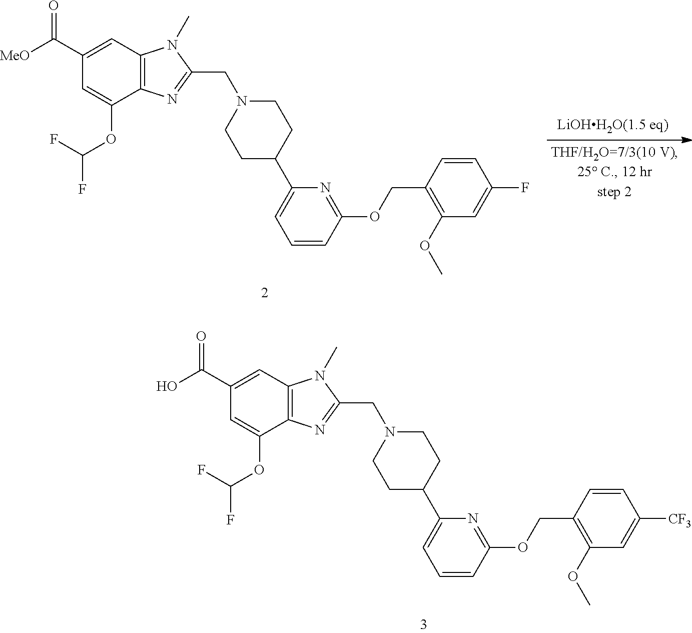 [SMILES: COC(=O)c1cc(OC(F)F)c2nc(CN3CCC(c4cccc(OCc5ccc(F)cc5OC)n4)CC3)n(C)c2c1 ; COc1cc(C(F)(F)F)ccc1COc1cccc(C2CCN(Cc3nc4c(OC(F)F)cc(C(=O)O)cc4n3C)CC2)n1]

To a solution of 2 (80 mg, 136.85 μmol, 1 eq) in THF (0.7 mL) and H₂O (0.3 mL) was added LiOH·H₂O (8.61 mg, 205.27 μmol, 1.5 eq). The mixture was stirred at 25° C. for 12 hr. LCMS (product: RT=1.180 min) showed the 2 was consumed completely. The crude product was purified by reversed-phase HPLC (column: Waters Xbridge Prep OBD C18 150*40 mm*10 um; mobile phase: [H₂O (10 mM NH₄HCO₃)-ACN]; gradient: 25%-75% B over 8.0 min). 4-(Difluoromethoxy)-2-((4-(6-((4-fluoro-2-methoxybenzyl)oxy)pyridin-2-yl)piperidin-1-yl)methyl)-1-methyl-1H-benzo[d]imidazole-6-carboxylic acid (24.89 mg, 43.62 μmol, 31.88% yield) was obtained as a white solid.

LCMS: RT=2.663 min, MS cal.: 570.6, [M−H]⁻=571.2

HPLC: RT=11.474 min, Purity=99.62%

¹H NMR (400 MHz, MeOH-d4) δ=8.09 (d, J=1.2 Hz, 1H), 7.68 (s, 1H), 7.56 (t, J=7.6 Hz, 1H), 7.44 (s, 1H), 7.37 (dd, J=7.2, 8.4 Hz, 1H), 7.25 (s, 1H), 7.07 (s, 1H), 6.81 (d, J=7.2 Hz, 1H), 6.78 (dd, J=2.4, 11.2 Hz, 1H), 6.66-6.58 (m, 2H), 5.30 (s, 2H), 4.01 (s, 5H), 3.84 (s, 3H), 3.13 (br d, J=11.2 Hz, 2H), 2.75-2.63 (m, 1H), 2.50-2.39 (m, 2H), 1.97-1.88 (m, 4H)

### Example A152—Preparation of Compound 232. 2-((4-(6-((4-Chloro-2-fluorobenzyl)oxy)pyridin-2-yl)piperidin-1-yl)methyl)-4-(difluoromethoxy)-1-methyl-6-(5-(trifluoromethyl)-4H-1,2,4-triazol-3-yl)-1H-benzo[d]imidazole (Compound 232) (3)

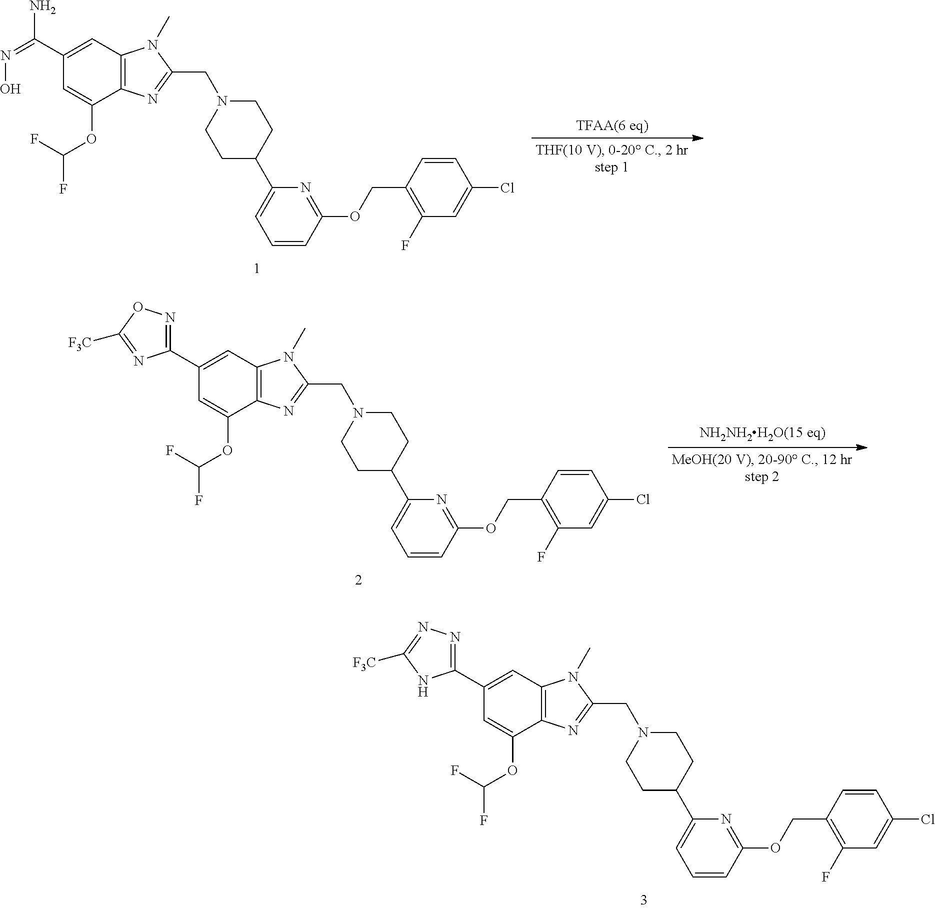 [SMILES: Cn1c(CN2CCC(c3cccc(OCc4ccc(Cl)cc4F)n3)CC2)nc2c(OC(F)F)cc(/C(N)=N\O)cc21 ; Cn1c(CN2CCC(c3cccc(OCc4ccc(Cl)cc4F)n3)CC2)nc2c(OC(F)F)cc(-c3noc(C(F)(F)F)n3)cc21 ; Cn1c(CN2CCC(c3cccc(OCc4ccc(Cl)cc4F)n3)CC2)nc2c(OC(F)F)cc(-c3nnc(C(F)(F)F)[nH]3)cc21]

### General Procedure for Preparation of Intermediate 2:

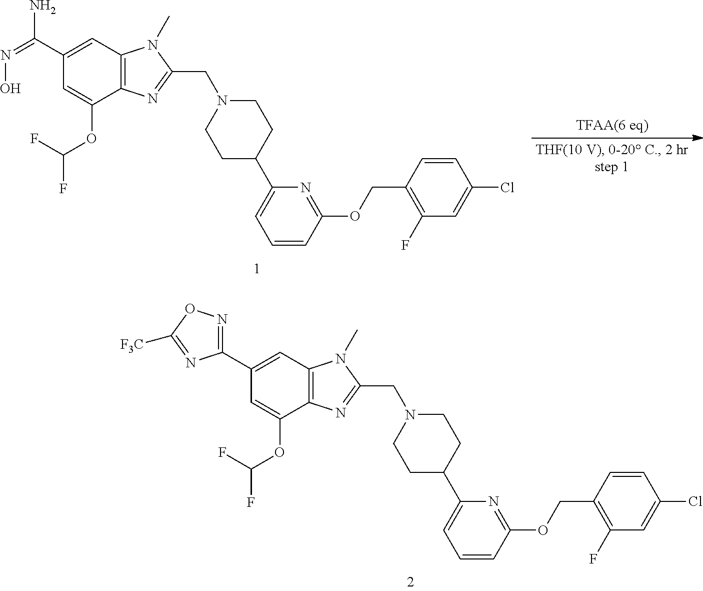 [SMILES: Cn1c(CN2CCC(c3cccc(OCc4ccc(Cl)cc4F)n3)CC2)nc2c(OC(F)F)cc(/C(N)=N\O)cc21 ; Cn1c(CN2CCC(c3cccc(OCc4ccc(Cl)cc4F)n3)CC2)nc2c(OC(F)F)cc(-c3noc(C(F)(F)F)n3)cc21]

A mixture of 1 (100 mg, 169.78 μmol, 1 eq) and TFAA (213.95 mg, 1.02 mmol, 141.60 μL, 6 eq), in THF (1 mL) at 0° C. and was degassed and purged with N₂ for 3 times, and then the mixture was stirred at 20° C. for 2 hr under N₂ atmosphere. LCMS (product: RT=1.945 min) showed the 1 was consumed completely. The reaction mixture was quenched by addition H₂O 3 mL at 20° C. and extracted with ethyl acetate 15 mL (5 mL*3). The combined organic layers were washed with brine 10 mL (5 mL*2), dried over Na₂SO₄, filtered, and concentrated under reduced pressure to give a residue. The residue was purified by column chromatography (SiO₂, Petroleum ether/Ethyl acetate=1/0 to 0/1). 2 (90 mg, crude) was obtained as a white solid.

¹H NMR (400 MHz, DMSO-d6) δ=8.20 (d, J=1.6 Hz, 1H), 7.73 (t, J=74 Hz, 1H), 7.65-7.59 (m, 2H), 7.56-7.52 (m, 1H), 7.45 (dd, J=2.0, 10.0 Hz, 1H), 7.28 (dd, J=2.0, 8.4 Hz, 1H), 6.87 (d, J=7.2 Hz, 1H), 6.67 (d, J=8.4 Hz, 1H), 5.36 (s, 2H), 4.02 (s, 3H), 3.88 (s, 2H), 2.96 (d, J=11.6 Hz, 2H), 2.63-2.56 (m, 1H), 2.24 (t, J=10.4 Hz, 2H), 1.84-1.66 (m, 4H)

### General Procedure for Preparation of Compound 232 (3):

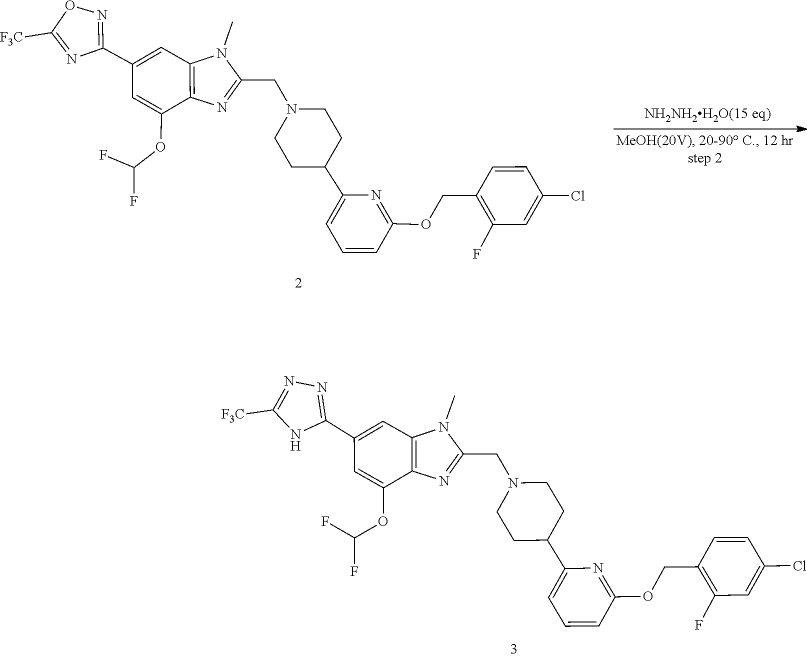 [SMILES: Cn1c(CN2CCC(c3cccc(OCc4ccc(Cl)cc4F)n3)CC2)nc2c(OC(F)F)cc(-c3noc(C(F)(F)F)n3)cc21 ; Cn1c(CN2CCC(c3cccc(OCc4ccc(Cl)cc4F)n3)CC2)nc2c(OC(F)F)cc(-c3nnc(C(F)(F)F)[nH]3)cc21]

MeOH (1.4 mL) was charged to the three-necked round bottom flask, then 2 (70 mg, 104.95 μmol, 1 eq) was added to the mixture at 20° C. NH₂NH₂·H₂O (78.81 mg, 1.57 mmol, 76.36 μL, 15 eq) was added dropwise to the reaction mixture at 20° C. After the addition, the mixture was stirred at 90° C. for 12 hr. LCMS (product: RT=1.501 min) showed the 2 was consumed completely. The reaction mixture was filtered. The mixture was added to H₂O (2 mL), extracted by ethyl acetate (3 mL*3), then the organic phase was combined and washed by brine 5 ml, dried with Na₂SO₄, filtered. The organic phase was concentrated under reduced pressure at 35° C. The residue was purified by prep-HPLC (column: Waters Xbridge Prep OBD C18 150*40 mm*10 um; mobile phase: [H₂O (10 mM NH₄HCO₃)—CH₃CN]; gradient: 45%-75% B over 8.0 min). 2-((4-(6-((4-Chloro-2-fluorobenzyl)oxy)pyridin-2-yl)piperidin-1-yl)methyl)-4-(difluoromethoxy)-1-methyl-6-(5-(trifluoromethyl)-4H-1,2,4-triazol-3-yl)-1H-benzo[d]imidazole (29.93 mg, 44.94 μmol, 42.82% yield, 100% purity) was obtained as a white solid.

LCMS: RT=3.328 min, MS cal.: 665.2, [M+H]⁺=666.2

HPLC: RT=13.995 min

¹H NMR (400 MHz, DMSO-d6) δ=8.16 (s, 1H), 7.71 (t, J=74.4 Hz, 1H), 7.66 (s, 1H), 7.62 (t, J=8.0 Hz, 1H), 7.55 (t, J=8.4 Hz, 1H), 7.45 (dd, J=2.0, 10.0 Hz, 1H), 7.28 (dd, J=1.6, 8.0 Hz, 1H), 6.87 (d, J=7.6 Hz, 1H), 6.67 (d, J=8.4 Hz, 1H), 5.36 (s, 2H), 3.99 (s, 3H), 3.87 (s, 2H), 2.96 (d, J=11.2 Hz, 2H), 2.65-2.55 (m, 1H), 2.24 (t, J=10.8 Hz, 2H), 1.84-1.64 (m, 4H)

### Example A153—Preparation of Compound 233. 2-((4-(6-((4-Chloro-2-fluorobenzyl)oxy)pyridin-2-yl)piperidin-1-yl)methyl)-4-(difluoromethoxy)-1-methyl-6-(5-(trifluoromethyl)-4H-1,2,4-triazol-3-yl)-1H-benzo[d]imidazole (Compound 233) (2)

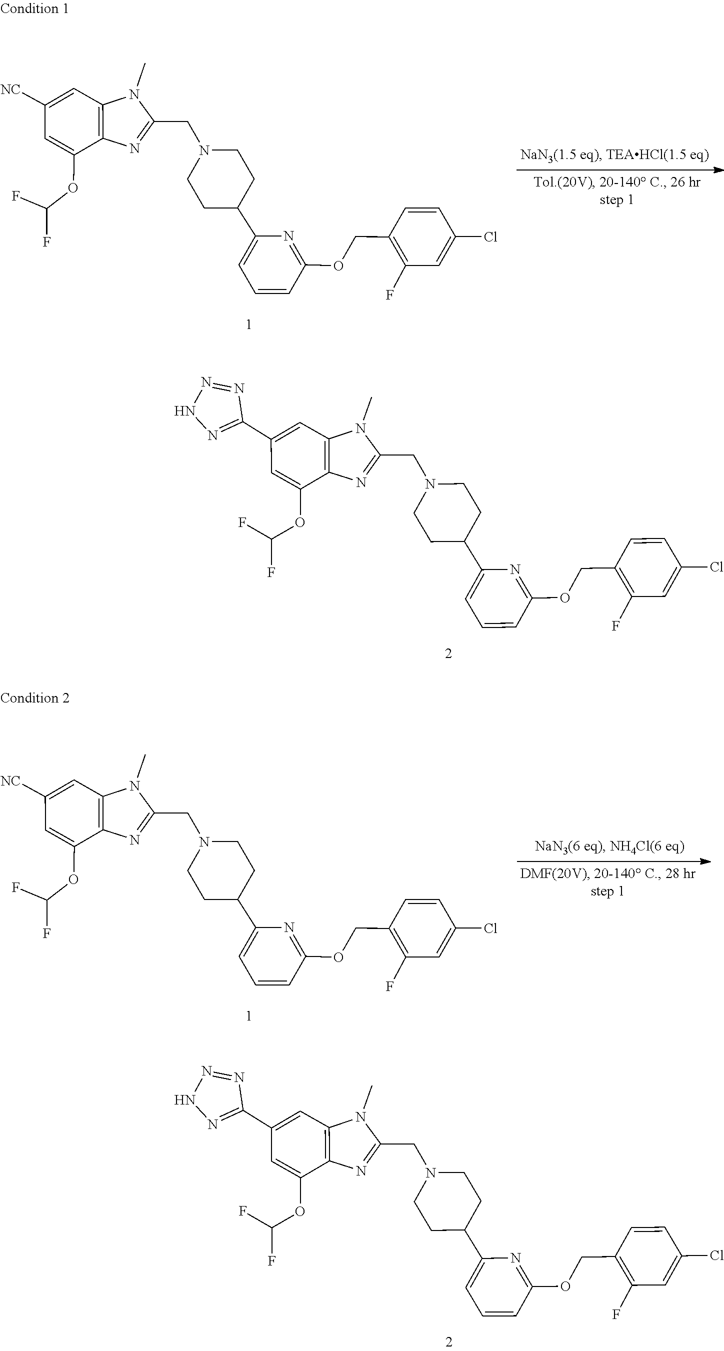 [SMILES: Cn1c(CN2CCC(c3cccc(OCc4ccc(Cl)cc4F)n3)CC2)nc2c(OC(F)F)cc(C#N)cc21 ; Cn1c(CN2CCC(c3cccc(OCc4ccc(Cl)cc4F)n3)CC2)nc2c(OC(F)F)cc(-c3nn[nH]n3)cc21 ; Cn1c(CN2CCC(c3cccc(OCc4ccc(Cl)cc4F)n3)CC2)nc2c(OC(F)F)cc(C#N)cc21 ; Cn1c(CN2CCC(c3cccc(OCc4ccc(Cl)cc4F)n3)CC2)nc2c(OC(F)F)cc(-c3nn[nH]n3)cc21]

### General Procedure for Preparation of Compound 233 (2):

### Condition 1:

Toluene (2 mL) was charged to the three-necked round bottom flask, then 1 (100 mg, 179.86 μmol, 1 eq) and TEA·HCl (37.14 mg, 269.80 μmol, 1.5 eq) was added to the mixture at 20° C. At 20° C., NaN₃ (17.54 mg, 269.80 μmol, 1.5 eq) was added in portions to the reaction mixture at 20° C. After the addition, the mixture was stirred at 80° C. for 12 hr. Then the reaction was heated to 120° C., stirred at 120° C. for 12 hr. Then the reaction was heated to 140° C., stirred at 140° C. for 2 hr. LCMS product: RT=1.213 min; start material: RT=1.693 min) showed 54% of 1 remained. Several new peaks were shown on LC-MS and 8% of desired Compound was detected. The reaction mixture was added sat. Na₂CO₃ adjust to pH=10-12 at 0° C. The mixture was extracted by EtOAc (5 mL*3). Then organic phase was combined and washed by H₂O (5 mL), brine (5 mL*2), dried with Na₂SO₄, filtered. The organic phase was concentrated under reduced pressure at 35° C. to give a residue. The residue was purified by column chromatography (SiO₂, Petroleum ether/Ethyl acetate=3/1 to Ethyl acetate/Methanol=1/1) to give a crude product.

### Condition 2:

DMF (2 mL) was charged to the three-necked round bottom flask, 1 (100 mg, 179.86 μmol, 1 eq) and NH₄Cl (28.86 mg, 539.59 μmol, 3 eq) was added to the mixture at 20° C. NaN₃ (35.08 mg, 539.59 μmol, 3 eq) was added in portions to the reaction mixture at 20° C. The mixture was stirred at 90° C. for 12 hr. Then the reaction was heated to 120° C., stirred at 120° C. for 2 hr. The reaction was cooled down to 20° C., NH₄Cl (28.86 mg, 539.59 μmol, 3 eq), NaN₃ (35.08 mg, 539.59 μmol, 3 eq) was added in portions to the reaction mixture. Then the reaction was heated to 120° C., stirred at 120° C. for 12 hr. Then the reaction was heated to 140° C., stirred at 140° C. for 2 hr. LCMS (ET64759-565-P1W, product: RT=1.210 min; start material: RT=1.689 min) showed 63% of 1 remained. On LC-MS and 16% of desired Compound was detected. The reaction mixture was added sat. Na₂CO₃ adjust to pH=10-12 at 0° C. The mixture was extracted by EtOAc (5 mL*3). Then organic phase was combined and washed by H₂O (5 mL), brine (5 mL*2), dried with Na₂SO₄, filtered. The organic phase was concentrated under reduced pressure at 35° C. to give a residue. The residue was purified by column chromatography (SiO₂, Petroleum ether/Ethyl acetate=3/1 to Ethyl acetate/Methanol=1/1) to give a crude product. The crude product was purified by prep-HPLC (column: Waters Xbridge Prep OBD C18 150*40 mm*10 um; mobile phase: [H₂O (10 mM NH₄HCO₃)-can]; gradient: 15%-70% B over 8.0 min). 2-((4-(6-((4-Chloro-2-fluorobenzyl)o790midazoldin-2-yl)piperidin-1-yl)methyl)-4-(difluoromethoxy)-1-methyl-6-(5-(trifluoromethyl)-4H-1,2,4-triazol-3-yl)-1H-benzo[d]imidazole (10.69 mg, 17.85 μmol, 53.45% yield, 100% purity) was obtained as a white solid.

LCMS: RT=2.884 min, MS cal.: 598.2, [M+H]=599.2

HPLC: RT=12.134 min

¹H NMR (400 MHz, DMSO-d6) δ=8.13 (d, J=1.2 Hz, 1H), 7.70 (t, J=74.4 Hz, 1H), 7.66 (s, 1H), 7.-5-7.61 (m, 1H), 7.56 (t, J=8.0 Hz, 1H), 7.46 (dd, J=2.0, 10.0 Hz, 1H), 7.29 (dd, J=2.0, 8.0 Hz, 1H), 6.88 (d, J=7.2 Hz, 1H), 6.68 (d, J=8.4 Hz, 1H), 5.36 (s, 2H), 3.98 (s, 5H), 3.-9-3.00 (m, 2H), 2.-0-2.61 (m, 1H), 2.-9-2.29 (m, 2H), 1.-8-1.71 (m, 4H)

### Example A154—Preparation of Compound 234. 5-(2-((4-(6-((4-Chloro-2-fluorobenzyl)o791midazoldin-2-yl)piperidin-1-yl)methyl)-4-(difluoromethoxy)-1-methyl-1H-benzo791midazolezol-6-yl)-1,3,4-oxadiazol-2 (3H)-one (Compound 234) (4)

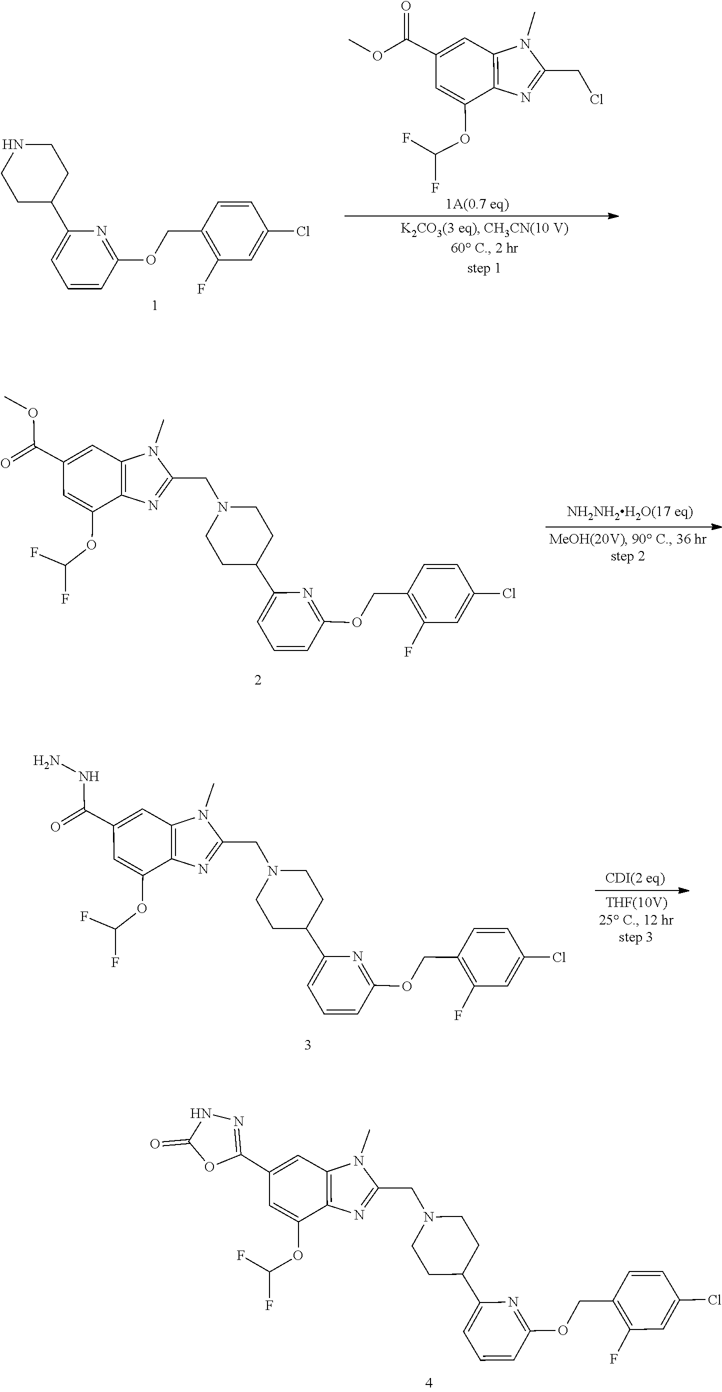 [SMILES: Fc1cc(Cl)ccc1COc1cccc(C2CCNCC2)n1 ; COC(=O)c1cc(OC(F)F)c2nc(CCl)n(C)c2c1 ; COC(=O)c1cc(OC(F)F)c2nc(CN3CCC(c4cccc(OCc5ccc(Cl)cc5F)n4)CC3)n(C)c2c1 ; Cn1c(CN2CCC(c3cccc(OCc4ccc(Cl)cc4F)n3)CC2)nc2c(OC(F)F)cc(C(=O)NN)cc21 ; Cn1c(CN2CCC(c3cccc(OCc4ccc(Cl)cc4F)n3)CC2)nc2c(OC(F)F)cc(-c3n[nH]c(=O)o3)cc21]

### General Procedure for Preparation of Intermediate 2:

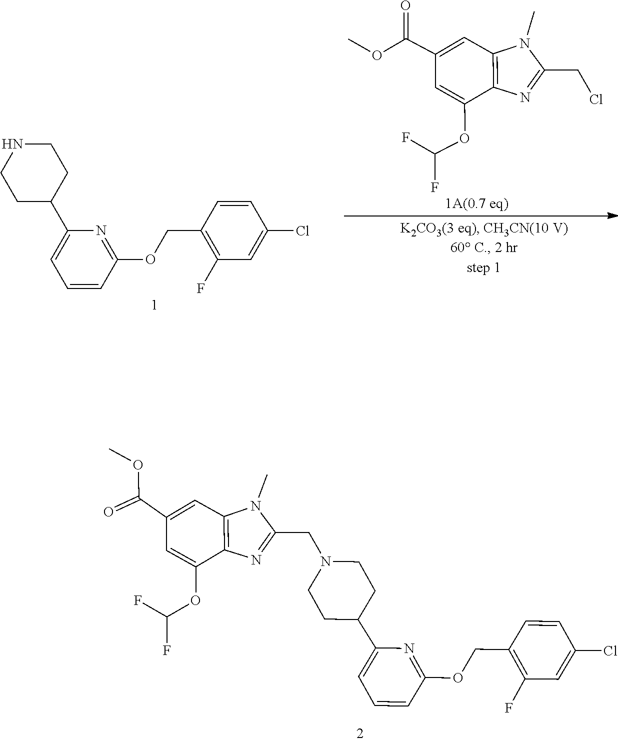 [SMILES: Fc1cc(Cl)ccc1COc1cccc(C2CCNCC2)n1 ; COC(=O)c1cc(OC(F)F)c2nc(CCl)n(C)c2c1 ; COC(=O)c1cc(OC(F)F)c2nc(CN3CCC(c4cccc(OCc5ccc(Cl)cc5F)n4)CC3)n(C)c2c1]

A mixture of 1A (132.97 mg, 436.42 μmol, 0.7 eq), 1 (200 mg, 623.46 μmol, 1 eq), K₂CO₃ (258.50 mg, 1.87 mmol, 3 eq) in CH₃CN (2 mL) was degassed and purged with N₂ for 3 times, and then the mixture was stirred at 60° C. for 2 hr under N₂ atmosphere. LCMS (product: RT=1.708 min) showed 1 was consumed completely. The reaction mixture was filtered and quenched by addition H₂O 3 mL at 20° C., and extracted with EtOAc 3 mL*3. The combined organic layers were washed with brine 5 mL*2, dried over Na₂SO₄, filtered and concentrated under reduced pressure to give a residue. The residue was purified by column chromatography (SiO₂, Petroleum ether/Ethyl acetate=1/1 to 0/1). 2 (220 mg, 373.51 μmol, 59.91% yield) was obtained as a white solid.

¹H NMR (400 MHz, MeOH-d4) δ=8.14 (s, 1H), 7.68 (s, 1H), 7.57 (t, J=8.0 Hz, 1H), 7.48 (t, J=8.4 Hz, 1H), 7.26 (t, J=74 Hz, 1H), 7.22-7.15 (m, 2H), 6.81 (d, J=7.6 Hz, 1H), 6.62 (d, J=8.4 Hz, 1H), 5.40 (s, 2H), 4.04 (s, 3H), 3.96 (s, 3H), 3.91 (s, 2H), 3.01 (d, J=11.6 Hz, 2H), 2.70-2.56 (m, 1H), 2.38-2.25 (m, 2H), 1.89-1.82 (m, 4H)

### General Procedure for Preparation of Intermediate 3:

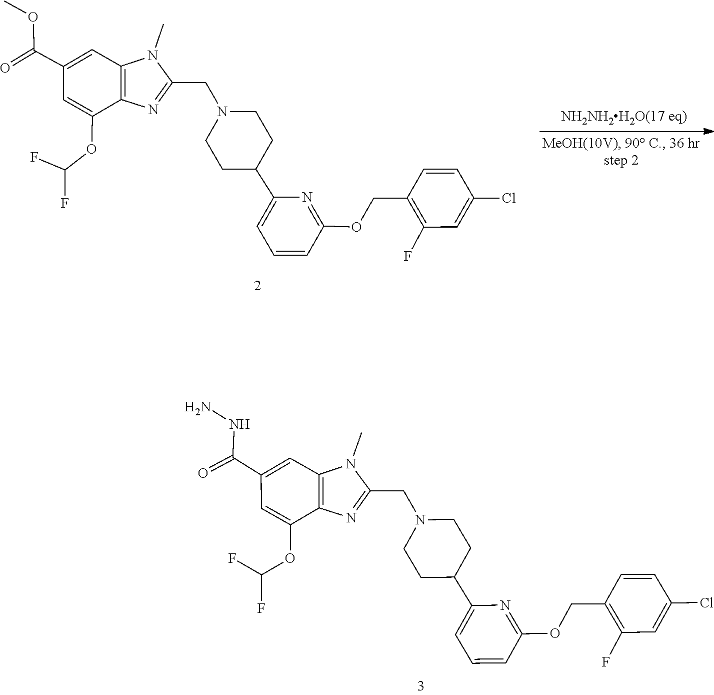 [SMILES: COC(=O)c1cc(OC(F)F)c2nc(CN3CCC(c4cccc(OCc5ccc(Cl)cc5F)n4)CC3)n(C)c2c1 ; Cn1c(CN2CCC(c3cccc(OCc4ccc(Cl)cc4F)n3)CC2)nc2c(OC(F)F)cc(C(=O)NN)cc21]

MeOH (1.5 mL) was charged to the three-necked round bottom flask, then 2 (150 mg, 254.67 μmol, 1 eq) was added to the mixture at 20° C. NH₂NH₂·H₂O (25.50 mg, 509.33 μmol, 24.71 μL, 2 eq) was added dropwise to the reaction mixture at 20° C. After the addition, the mixture was stirred at 90° C. for 12 hr. The reaction mixture was cooled down to 20° C., NH₂NH₂·H₂O (63.74 mg, 1.27 mmol, 61.77 μL, 5 eq) was added dropwise to the reaction mixture. After the addition, the mixture was stirred at 90° C. for 12 hr. The reaction mixture was cooled down to 20° C., NH₂NH₂·H₂O (127.49 mg, 2.55 mmol, 123.53 μL, 10 eq) was added dropwise to the reaction mixture. After the addition, the mixture was stirred at 90° C. for 12 hr. LCMS (product: RT=1.412 min) showed the 2 was consumed completely. The reaction mixture was cooled down to 20° C. and filtered. The filtrate was added to H₂O (2 mL), extracted by EtOAc (3 mL*3), then organic phase was combined and washed by brine 5 ml, dried with Na₂SO₄, filtered. The organic phase was concentrated under reduced pressure at 35° C. 3 (100 mg, 169.78 μmol, 66.67% yield) was obtained as a white solid.

### General Procedure for Preparation of Compound 234 (4):

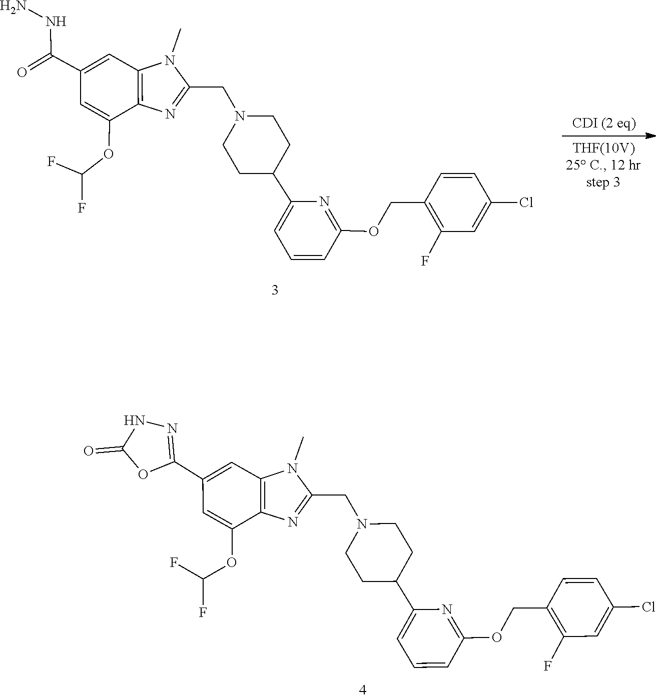 [SMILES: Cn1c(CN2CCC(c3cccc(OCc4ccc(Cl)cc4F)n3)CC2)nc2c(OC(F)F)cc(C(=O)NN)cc21 ; Cn1c(CN2CCC(c3cccc(OCc4ccc(Cl)cc4F)n3)CC2)nc2c(OC(F)F)cc(-c3n[nH]c(=O)o3)cc21]

To a solution of 3 (30 mg, 50.93 μmol, 1 eq) in THF (0.3 mL) was added CDI (16.52 mg, 101.87 μmol, 2 eq). The mixture was stirred at 25° C. for 12 hr. LCMS (product: RT=1.485 min) showed the 3 was consumed completely. The reaction mixture was filtered. The filtrate was purified by prep-HPLC (column: Waters Xbridge Prep OBD C18 150*40 mm*10 um; mobile phase: [H₂O (10 mM NH₄HCO₃)-ACN]; gradient: 20%-70% B over 8.0 min). 5-(2-((4-(6-((4-Chloro-2-fluorobenzyl)oxy)pyridin-2-yl)piperidin-1-yl)methyl)-4-(difluoromethoxy)-1-methyl-1H-benzo[d]imidazol-6-yl)-1,3,4-oxadiazol-2 (3H)-one (10.79 mg, 17.44 μmol, 34.23% yield, 99.38% purity) was obtained as a white solid.

LCMS: RT=3.374 min, MS cal.: 614.2, [M+H]⁺=615.2

HPLC: RT=12.235 min

¹H NMR (400 MHz, DMSO-d6) δ=12.70-12.56 (m, 1H), 7.92 (d, J=1.2 Hz, 1H), 7.69 (t, J=74.4 Hz, 1H), 7.62 (t, J=7.6 Hz, 1H), 7.58-7.52 (m, 1H), 7.45 (dd, J=2.0, 10.4 Hz, 1H), 7.36 (s, 1H), 7.31-7.25 (m, 1H), 6.87 (d, J=7.2 Hz, 1H), 6.67 (d, J=8.4 Hz, 1H), 5.36 (s, 2H), 3.98 (s, 3H), 3.85 (s, 2H), 2.94 (d, J=11.4 Hz, 2H), 2.62 (br d, J=5.6 Hz, 1H), 2.27-2.17 (m, 2H), 1.82-1.65 (m, 4H)

### Example A155—Preparation of Compound 235. 2-((4-(6-((4-Chloro-2-fluorobenzyl)oxy)pyridin-2-yl)piperidin-1-yl)methyl)-1-methyl-4-(1,1,2-trifluoroethoxy)-1H-benzo[d]imidazole-6-carboxylic acid (Compound 235) (12)

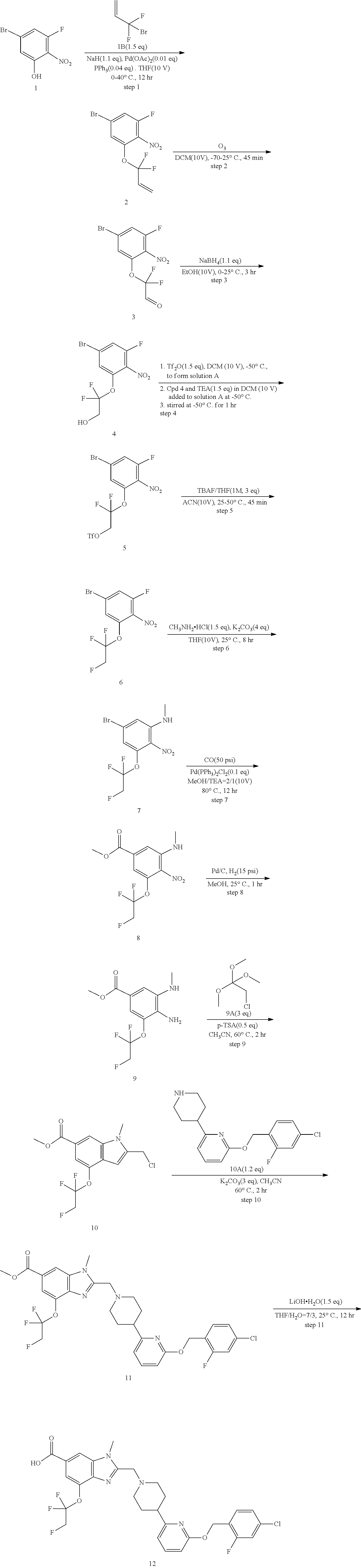

### General Procedure for Preparation of Intermediate 2:

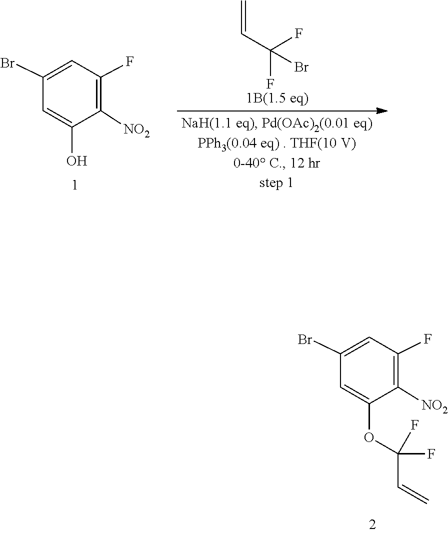

Equip a 250 mL three-necked round bottom flask, thermometer, N₂ balloon. THF (45 mL) was charged to the three-necked round bottom flask, then 1 (5 g, 21.19 mmol, 1 eq,) was added to the mixture at 25° C. At 0° C. inner temperature, NaH (932.13 mg, 23.31 mmol, 60% purity, 1.1 eq) was added to the reaction mixture at 0° C. After the addition, the mixture was stirred at 0° C. for 5 min, and then Pd(OAc)₂ (47.57 mg, 211.87 μmol, 0.01 eq) and PPh₃ (222.28 mg, 847.47 μmol, 0.04 eq) was added to the mixture. At 0° C. inner temperature, 1B (4.99 g, 31.78 mmol, 1.5 eq) in THF (5 mL) was added to the reaction mixture at 0° C. After the addition, the mixture was stirred at 40° C. for 12 hr. The reaction was monitored by TLC. TLC (Petroleum ether:Ethyl acetate=3:1, R_{f}=0.68) indicated ˜0% of 1 was remained, and one major new spot with lower polarity was detected. The reaction mixture was added to H₂O (50 mL) at 0° C. The mixture was extracted by EtOAc (50 mL*3). Then organic phase was combined and washed by brine (20 mL), dried with Na₂SO₄, filtered. The organic phase was concentrated under reduced pressure at 40° C. to give a residue. The residue was purified by column chromatography (SiO₂, Petroleum ether/Ethyl acetate=100/1 to 3/1). 2 (6.31 g, 20.22 mmol, 95.44% yield) was obtained as a yellow oil.

¹H NMR (400 MHz, DMSO-d₆) δ=8.08 (dd, J=2.0, 9.6 Hz, 1H), 7.74 (s, 1H), 6.34-6.21 (m, 1H), 5.96 (td, J=2.4 17.2 Hz, 1H), 5.85 (d, J=10.8 Hz, 1H)

### General Procedure for Preparation of Intermediate 3:

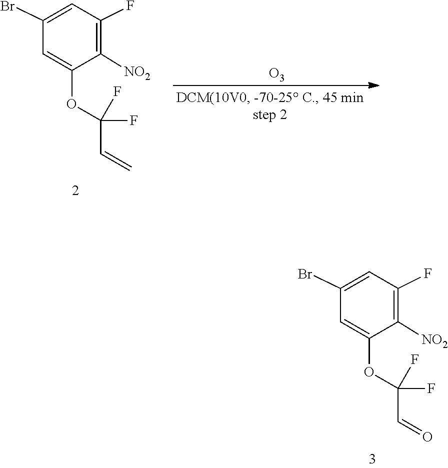 [SMILES: C=CC(F)(F)Oc1cc(Br)cc(F)c1[N+](=O)[O-] ; "O3" ; O=CC(F)(F)Oc1cc(Br)cc(F)c1[N+](=O)[O-]]

Equip a 250 mL three-necked round bottom flask, thermometer. DCM (60 mL) was charged to the 250 mL three-necked round bottom flask, then 2 (6.31 g, 20.22 mmol, 1 eq) was added at 25° C. After the addition, cooled the reaction mixture to −70° C. At −70° C. inner temperature, O₃ gas was carefully bubbled into the reactor for 15 mins so that the temperature of the exothermic reaction did not exceed −70° C. The mixture was monitored by TLC. TLC (Petroleum ether:Ethyl acetate=3:1, R_{f}=0.48) indicated 2 was consumed, and one major new spot with larger polarity was detected. 3 (6.35 g, crude) was used to next step in the DCM solution without purification.

### General Procedure for Preparation of Intermediate 4:

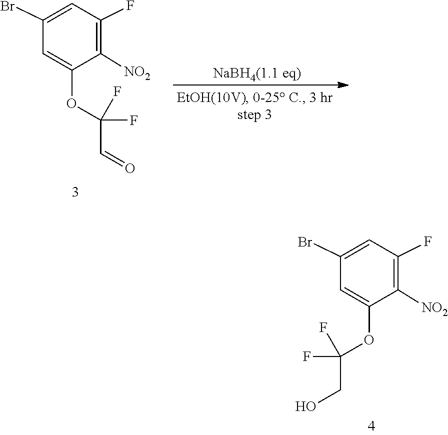 [SMILES: O=CC(F)(F)Oc1cc(Br)cc(F)c1[N+](=O)[O-] ; "NaBH4(1.1 eq)" ; O=[N+]([O-])c1c(F)cc(Br)cc1OC(F)(F)CO]

To a solution of 3 (6.35 g, 20.22 mmol, 1 eq) in DCM (60 mL) was added EtOH (6 mL) at 25° C. At 0° C. inner temperature, NaBH₄ (841.50 mg, 22.24 mmol, 1.1 eq) was added in portions to the reaction mixture at 0° C. After the addition, the mixture was stirred at 25° C. for 3 hr. The mixture was monitered by TLC. TLC (Petroleum ether:Ethyl acetate=3:1, R_{f}=0.43) indicated 3 was consumed, and one major new spot with larger polarity was detected. The reaction mixture was added to H₂O (50 mL) at 0° C. The mixture was extracted by DCM (60 mL*3). Then organic phase was combined and washed by brine (30 mL), dried with Na₂SO₄, filtered. The organic phase was concentrated under reduced pressure at 40° C. to give a residue. The residue was purified by column chromatography (SiO₂, Petroleum ether/Ethyl acetate=100/1 to 3/1). 4 (2.42 g, 7.66 mmol, 37.87% yield) was obtained as a yellow oil.

¹H NMR (400 MHz, CHCl3-d) δ=7.51 (s, 1H), 7.38 (dd, J=1.6, 8.4 Hz, 1H), 4.11-3.93 (m, 2H), 2.20 (br t, J=7.2 Hz, 1H)

### General Procedure for Preparation of Intermediate 5:

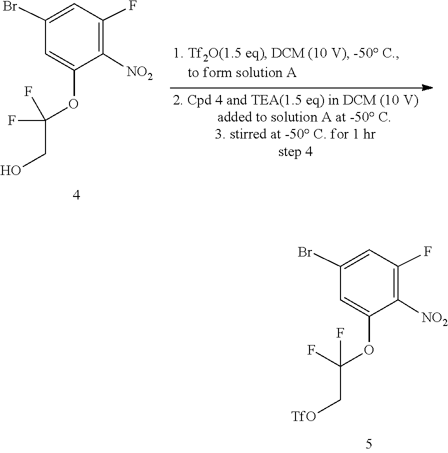 [SMILES: O=[N+]([O-])c1c(F)cc(Br)cc1OC(F)(F)CO ; O=[N+]([O-])c1c(F)cc(Br)cc1OC(F)(F)COS(=O)(=O)C(F)(F)F]

Equip a 50 mL three-necked round bottom flask, and thermometer, N₂ balloon (Reaction A). DCM (10 mL) was charged to the 50 mL three-necked round bottom flask, then 4 (1 g, 3.16 mmol, 1 eq) was added at 25° C. under N₂. At −10° C. (inner temperature), TEA (329.80 mg, 3.26 mmol, 453.64 μL, 1.03 eq) was added in portions to the reaction mixture at −10° C. form solution A. Equip another 50 mL three-necked round bottom flask, and thermometer, N₂ balloon (Reaction B). DCM (10 mL) was charged to the 50 mL three-necked round bottom flask, then Tf₂O (1.34 g, 4.75 mmol, 783.13 μL, 1.5 eq) was added at 25° C. under N₂ to form solution B. Solution B was cooled to −50° C., and then solution A was added to the solution B at −50° C., after addition, the mixture was stirred at −50° C. for 1 hr. The mixture was monitored by TLC. TLC (Petroleum ether:Ethyl acetate=5:1, R_{f}=0.54) indicated ˜20% of 4 was remained, and one major new spot with lower polarity was detected. The reaction mixture was diluted with H₂O (10 mL) and extracted with DCM (10 mL*3). The combined organic layers were washed with brine (10 mL*2), dried over Na₂SO₄, filtered, and concentrated under reduced pressure to give a residue. The crude product on notebook page ET53089-986 (0.6 g) was combined to ET53089-987 for further purification. The residue was purified by column chromatography (SiO₂, Petroleum ether/Ethyl acetate=100/1 to 1/1). 5 (1.48 g, crude) was obtained as a yellow oil.

¹H NMR (400 MHz, CHCl3-d) δ=7.49 (s, 1H), 7.45 (dd, J=1.6, 8.4 Hz, 1H), 4.78 (t, J=8.4 Hz, 2H)

### General Procedure for Preparation of Intermediate 6:

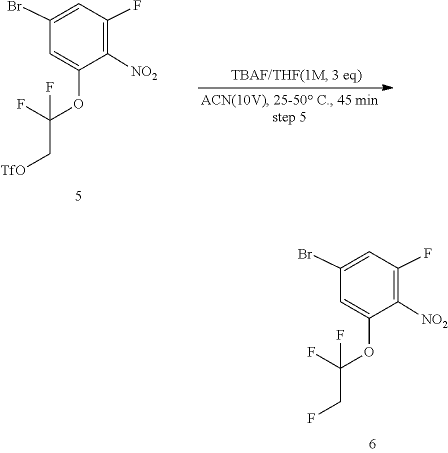 [SMILES: O=[N+]([O-])c1c(F)cc(Br)cc1OC(F)(F)COS(=O)(=O)C(F)(F)F ; O=[N+]([O-])c1c(F)cc(Br)cc1OC(F)(F)CF]

Equip a 25 mL three-necked bottom flask, and N₂ balloon. MeCN (6 mL) was charged to the 25 mL three-necked round bottom flask, 5 (600 mg, 1.34 mmol, 1 eq) was added at 25° C. At 25° C. (inner temperature), TBAF (1 M, 4.02 mL, 3 eq) was added to the reaction mixture and then stirred at 25° C. for 15 min. Then, the mixture was stirred at 50° C. for 30 min. TLC (Petroleum ether:Ethyl acetate=10:1, R_{f}=0.45) indicated 5 was consumed completely. The reaction mixture was diluted with H₂O 20 mL and extracted with EtOAc (20 mL*3). The combined organic layers were washed with brine (15 mL), dried over Na₂SO₄, filtered, and concentrated under reduced pressure to give a residue. The residue was purified by column chromatography (SiO₂, Petroleum ether/Ethyl acetate=100/1 to 90/1). 6 (730 mg, 2.30 mmol, 85.71% yield) was obtained as a yellow oil.

¹H NMR (400 MHz, CHCl3-d) δ=7.50 (s, 1H), 7.41 (dd, J=1.6, 8.4 Hz, 1H), 4.74 (t, J=9.2 Hz, 1H), 4.62 (t, J=8.8 Hz, 1H)

### General Procedure for Preparation of Intermediate 7:

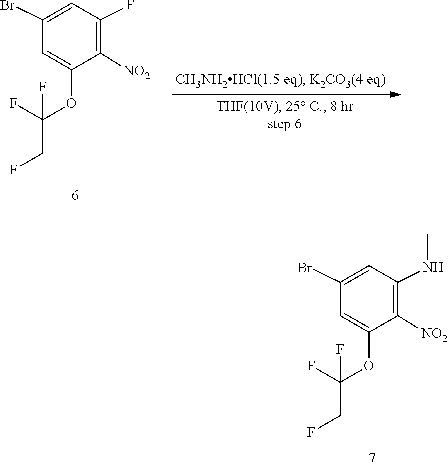 [SMILES: O=[N+]([O-])c1c(F)cc(Br)cc1OC(F)(F)CF ; CNc1cc(Br)cc(OC(F)(F)CF)c1[N+](=O)[O-]]

Equip a 25 mL three-necked bottom flask, and N₂ balloon. THF (7 mL) was charged to the 25 mL three-necked round bottom flask, 6 (630 mg, 1.98 mmol, 1 eq) and K₂CO₃ (1.10 g, 7.92 mmol, 4 eq) was added at 25° C. At 25° C. (inner temperature), CH₃NH₂ HCl (200.63 mg, 2.97 mmol, 1.5 eq) was added to the reaction mixture. After the addition, the mixture was stirred at 25° C. for 8 hr. TLC (Petroleum ether:Ethyl acetate=5:1, R_{f}=0.40) indicated 6 was consumed, and one major new spot with larger polarity was detected. The reaction mixture was added FA (1 M) to pH=5 at 25° C. The mixture was extracted by EtOAc (10 mL*3). Then organic phase was combined and washed by H₂O 10 mL, brine 10 mL, dried with Na₂SO₄, filtered. The organic phase was concentrated under reduced pressure at 45° C. to give a residue. The residue was purified by column chromatography (SiO₂, Petroleum ether/Ethyl acetate=100/1 to 1/1). 7 (710 mg, crude) was obtained as a yellow solid.

LCMS: RT=0.850 min, MS cal.: 329.1, [M+H]⁺=329.0

¹H NMR (400 MHz, CHCl3-d) δ=6.88 (d, J=1.6 Hz, 1H), 6.86 (s, 1H), 6.67-6.53 (m, 1H), 4.76 (t, J=9.2 Hz, 1H), 4.64 (t, J=9.2 Hz, 1H), 2.96 (d, J=3.6 Hz, 3H)

### General Procedure for Preparation of Intermediate 8:

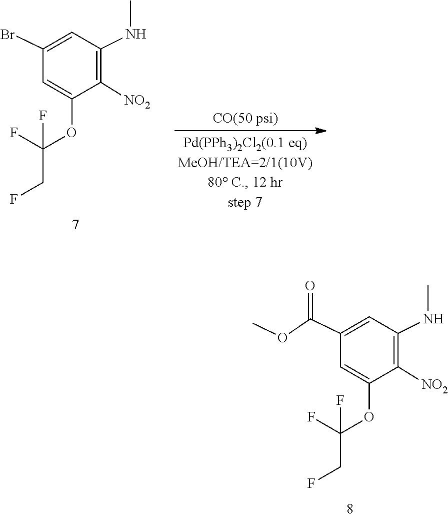

Equip a 35 ml Hydrogenated bottle. MeOH (0.6 mL) and TEA (0.3 mL) was charged to the 35 mL Hydrogenated bottle flask, then 7 (100 mg, 303.89 μmol, 1 eq), Pd(PPh₃)₂Cl₂ (21.33 mg, 30.39 μmol, 0.1 eq) was added at 25° C. After the addition, purged with CO for 3 times the mixture was stirred at 80° C. (50 psi) for 12 hr. The mixture was monitored by TLC and LCMS. TLC (Petroleum ether:Ethyl acetate=3:1, R_{f}=0.34) indicated 7 was consumed, and one major new spot with lower polarity was detected. The reaction mixture was concentrated under reduced pressure to remove MeOH. The residue was diluted with H₂O (10 mL) and extracted with EtOAc (10 mL*3). The combined organic layers were washed with brine (10 mL), dried over Na₂SO₄, filtered, and concentrated under reduced pressure to give a residue. The residue was purified by column chromatography (SiO₂, Petroleum ether/Ethyl acetate=100/1 to 3/1). 8 (60 mg, 194.67 μmol, 64.06% yield) was obtained as a yellow solid.

¹H NMR (400 MHz, CHCl3-d) δ=7.41 (d, J=1.2 Hz, 1H), 7.31 (d, J=1.2 Hz, 1H), 6.21 (br s, 1H), 4.76 (t, J=9.2 Hz, 1H), 4.64 (t, J=9.2 Hz, 1H), 3.96 (s, 3H), 3.05-2.98 (m, 3H)

### General Procedure for Preparation of Intermediate 9:

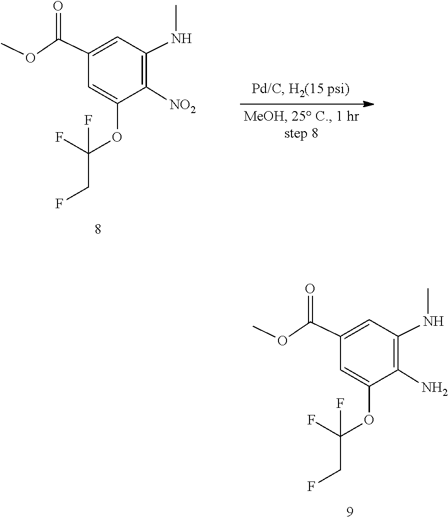

Equip a 10 mL round bottom flask, H₂ (15 psi) atmosphere. MeOH (0.6 mL) was charged to the 10 mL round bottom flask, then 8 (60 mg, 194.67 μmol, 1 eq) was added to the mixture at 25° C. Pd/C (20.72 mg) in MeOH (0.6 mL) was added to the reaction mixture at 25° C. under Ar atmosphere. After the addition, the suspension was degassed and purged with H₂ for 3 times, and then the mixture was stirred under H₂ atmosphere at 25° C. for 1 hr. The mixture was monitored by TLC and LCMS. LCMS (product: RT=1.072 min) showed the 8 was consumed completely. The reaction mixture was diluted with MeOH (10 ml), and then filtered through celite pad. The filter cake was rinsed with MeOH (10 mL*3), and the filtrate was concentrated under reduced pressure to give a product. 9 (50 mg, crude) was obtained as a yellow solid.

LCMS: RT=1.078 min, MS cal.: 278.2, [M+H]⁺=279.0

¹H NMR (400 MHz, CHCl3-d) δ=7.49 (br s, 1H), 7.30 (s, 1H), 4.80-4.72 (m, 1H), 4.65 (dt, J=2.0, 7.6 Hz, 1H), 3.90-3.86 (m, 3H), 2.94 (d, J=2.0 Hz, 3H)

### General Procedure for Preparation of Intermediate 10:

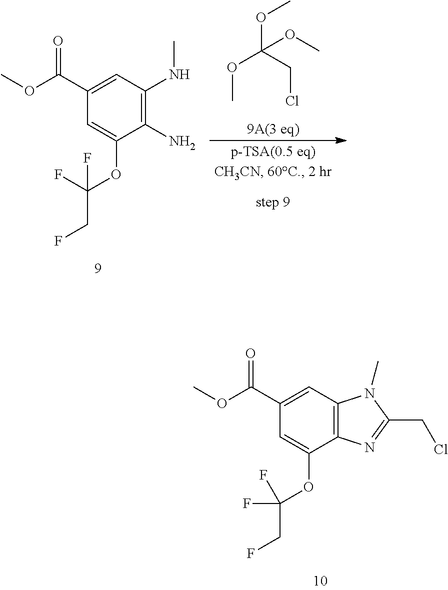

To a solution of 9 (50 mg, 179.71 μmol, 1 eq) and 9A (83.34 mg, 539.13 μmol, 72.66 μL, 3 eq) in ACN (1 mL) was added p-TSA (15.47 mg, 89.85 μmol, 0.5 eq). The mixture was stirred at 60° C. for 2 hr. LCMS (product: RT=1.116 min) showed the 9 was consumed completely. The reaction mixture was concentrated under reduced pressure to give a residue. 10 (60 mg, crude) was obtained as a yellow oil.

LCMS: RT=1.116 min, MS cal.: 336.7, [M+H]⁺=337.0

### General Procedure for Preparation of Intermediate 11:

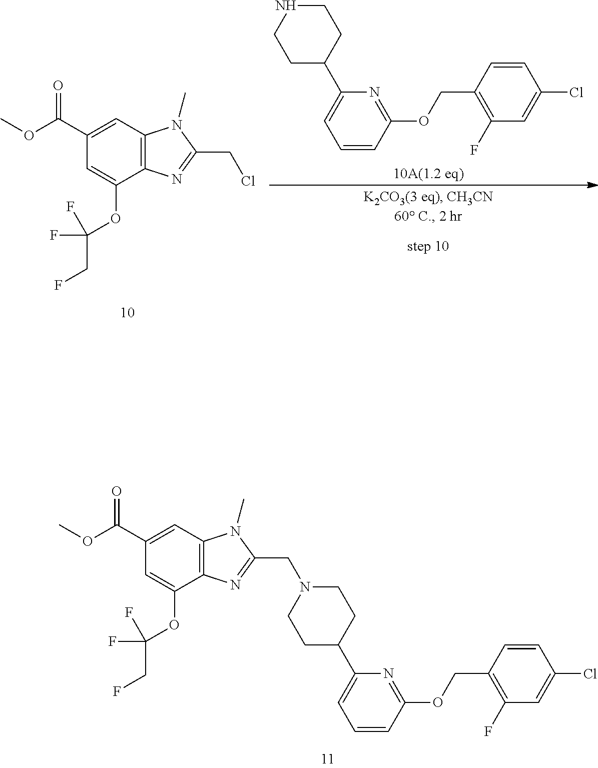 [SMILES: COC(=O)c1cc(OC(F)(F)CF)c2nc(CCl)n(C)c2c1 ; Fc1cc(Cl)ccc1COc1cccc(C2CCNCC2)n1 ; COC(=O)c1cc(OC(F)(F)CF)c2nc(CN3CCC(c4cccc(OCc5ccc(Cl)cc5F)n4)CC3)n(C)c2c1]

A mixture of 10 (60 mg, 178.20 μmol, 1 eq), 10A (68.60 mg, 213.84 μmol, 1.2 eq), K₂CO₃ (73.89 mg, 534.61 μmol, 3 eq) in ACN (1 mL) was degassed and purged with N₂ for 3 times, and then the mixture was stirred at 60° C. for 2 hr under N₂ atmosphere. LCMS (product: RT=1.683 min) showed the 10 was consumed completely. The reaction mixture was diluted with H₂O 10 mL and extracted with EtOAc (15 mL*3). The combined organic layers were washed with brine (10 mL*2), dried over Na₂SO₄, filtered and concentrated under reduced pressure to give a residue. The residue was purified by column chromatography (SiO₂, Petroleum ether/Ethyl acetate=10/1 to 0/1). 11 (60 mg, 96.62 μmol, 54.22% yield) was obtained as a yellow solid.

LCMS: RT=0.530 min, MS cal.: 621.0, [M+H]⁺=621.2

¹H NMR (400 MHz, CHCl3-d) δ=8.05 (s, 1H), 7.91 (s, 1H), 7.55-7.48 (m, 1H), 7.47-7.39 (m, 1H), 7.16-7.07 (m, 2H), 6.74 (d, J=7.2 Hz, 1H), 6.61 (d, J=8.0 Hz, 1H), 5.41 (s, 2H), 4.92 (t, J=9.2 Hz, 1H), 4.81 (t, J=9.2 Hz, 1H), 4.02 (br s, 3H), 3.98 (s, 3H), 3.92 (br s, 2H), 3.05-2.89 (m, 2H), 2.68-2.56 (m, 1H), 2.39-2.24 (m, 2H), 1.98-1.75 (m, 4H)

### General Procedure for Preparation of Compound 235 (12):

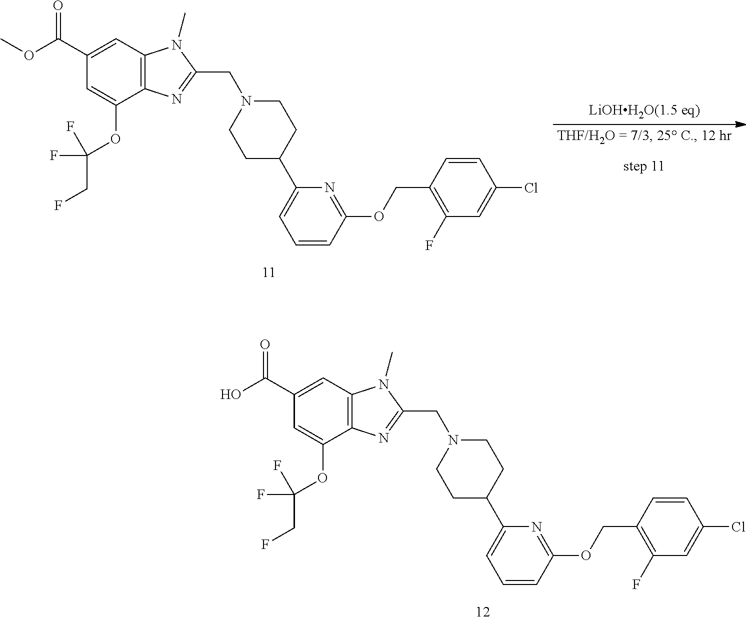 [SMILES: COC(=O)c1cc(OC(F)(F)CF)c2nc(CN3CCC(c4cccc(OCc5ccc(Cl)cc5F)n4)CC3)n(C)c2c1 ; Cn1c(CN2CCC(c3cccc(OCc4ccc(Cl)cc4F)n3)CC2)nc2c(OC(F)(F)CF)cc(C(=O)O)cc21]

To a solution of 11 (60 mg, 96.62 μmol, 1 eq) in THF (0.7 mL) and H₂O (0.3 mL) was added LiOH·H₂O (6.08 mg, 144.92 μmol, 1.5 eq). The mixture was stirred at 25° C. for 12 hr. LCMS (product: RT=1.183 min) showed the 11 was consumed completely. The crude product was purified by reversed-phase HPLC (column: Waters Xbridge Prep OBD C18 150*40 mm*10 um; mobile phase: [H₂O (10 mM NH₄HCO₃)-ACN]; gradient: 20%-60% B over 8.0 min). 2-((4-(6-((4-Chloro-2-fluorobenzyl)oxy)pyridin-2-yl)piperidin-1-yl)methyl)-1-methyl-4-(1,1,2-trifluoroethoxy)-1H-benzo[d]imidazole-6-carboxylic acid (25.15 mg, 41.43 μmol, 42.89% yield) was obtained as a white solid.

LCMS: RT=2.786 min, MS cal.: 607.0, [M+H]⁺=607.2

HPLC: RT=12.369 min, purity=99.613%

¹H NMR (400 MHz, DMSO-d₆) δ=8.13 (s, 1H), 7.67 (s, 1H), 7.62 (t, J=7.6 Hz, 1H), 7.55 (t, J=8.0 Hz, 1H), 7.45 (dd, J=1.6, 10.0 Hz, 1H), 7.28 (d, J=8.4 Hz, 1H), 6.86 (d, J=7.2 Hz, 1H), 6.66 (d, J=8.0 Hz, 1H), 5.35 (s, 2H), 5.13 (t, J=9.6 Hz, 1H), 5.02 (t, J=9.6 Hz, 1H), 3.97 (s, 3H), 3.85 (s, 2H), 2.92 (br d, J=10.8 Hz, 2H), 2.64-2.53 (m, 1H), 2.21 (br t, J=10.8 Hz, 2H), 1.82-1.64 (m, 4H)

### Example A156—Preparation of Compound 236. 3-(2-((4-(6-((4-Chloro-2-fluorobenzyl)oxy)pyridin-2-yl)piperidin-1-yl)methyl)-4-(difluoromethoxy)-1-methyl-1H-benzo[d]imidazol-6-yl)-1,2,4-oxadiazol-5 (4H)-one (Compound 236) (7)

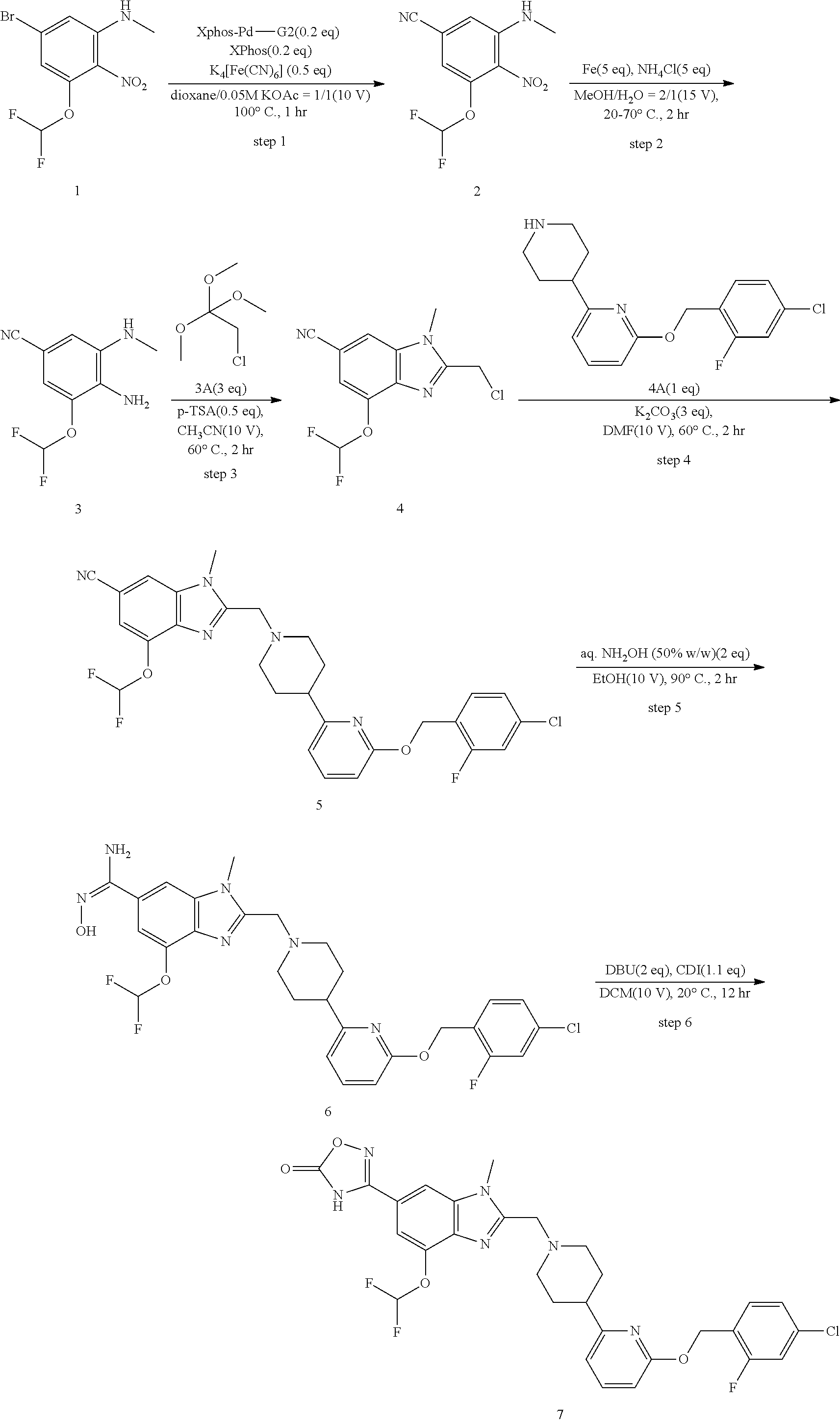

### General Procedure for Preparation of Intermediate 2:

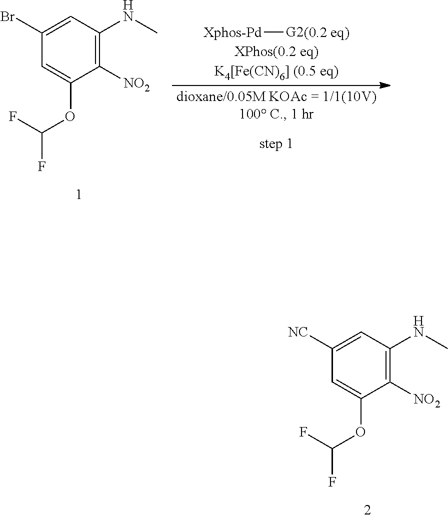

Dioxane (19 mL) and KOAc (19 mL, 0.05M) was charged to a three-necked round bottom flask, then 1 (3.8 g, 12.79 mmol, 1 eq) K₄[Fe(CN)₆] (2.36 g, 6.40 mmol, 0.5 eq) and XPhos (1.22 g, 2.56 mmol, 0.2 eq) was added to the mixture at 25° C. Xphos-Pd-G₂ (2.01 g, 2.56 mmol, 0.2 eq) was added to the reaction mixture at 25° C. After the addition, the mixture was stirred at 100° C. for 1 hr under N₂ atmosphere. LCMS (product: RT=1.131 min) showed the 1 was consumed completely. The reaction mixture was cooled down to 20° C., filtered, the organic phase and quenched by addition H₂O 50 mL at 20° C., and extracted with EtOAc 100 mL*3. The combined organic layers were washed with brine 50 mL*2, dried over Na₂SO₄, filtered, and concentrated under reduced pressure to give a residue. The residue was purified by column chromatography (SiO₂, Petroleum ether/Ethyl acetate=100/1 to 0/1). 2 (3 g, crude) was obtained as a red solid.

¹H NMR (400 MHz, CHCl3-d) δ=6.95 (d, J=1.2 Hz, 1H), 6.83 (d, J=1.2 Hz, 1H), 6.57 (t, J=72.4 Hz, 1H), 6.20-6.09 (m, 1H), 2.99 (d, J=4.8 Hz, 3H)

### General Procedure for Preparation of Intermediate 3:

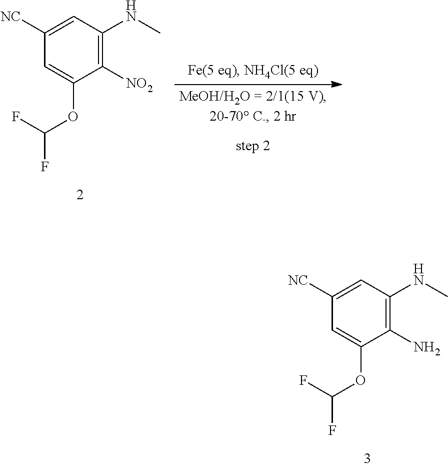 [SMILES: CNc1cc(C#N)cc(OC(F)F)c1[N+](=O)[O-] ; CNc1cc(C#N)cc(OC(F)F)c1N]

MeOH (25 mL) and H₂O (12.5 mL) was charged to the round bottom flask, then 2 (2.5 g, 10.28 mmol, 1 eq) and NH₄Cl (2.75 g, 51.41 mmol, 5 eq) was added to the mixture at 20° C. Fe (2.87 g, 51.41 mmol, 5 eq) was added in portions to the reaction mixture at 20° C. After the addition, the mixture was stirred at 70° C. for 2 hr. LCMS (product: RT=0.408 min) showed the 2 was consumed completely. The mixture was filtered, then the filtered cake was washed by MeOH 10 mL*6. The combined filtrate was added H₂O 50 mL and extracted with EtOAc 30 mL*3. Then organic phase was combined and washed by H₂O 15 mL, brine 20 mL*2, dried with Na₂SO₄, filtered. The organic phase was concentrated under reduced pressure at 35° C. to give a residue. 3 (2.5 g, crude) was obtained as a red solid.

¹H NMR (400 MHz, CHCl3-d) δ=6.94 (s, 1H), 6.77 (d, J=1.6 Hz, 1H), 6.50 (t, J=73.2 Hz, 1H), 3.96-3.62 (m, 2H), 2.90 (s, 3H)

### General Procedure for Preparation of Intermediate 4:

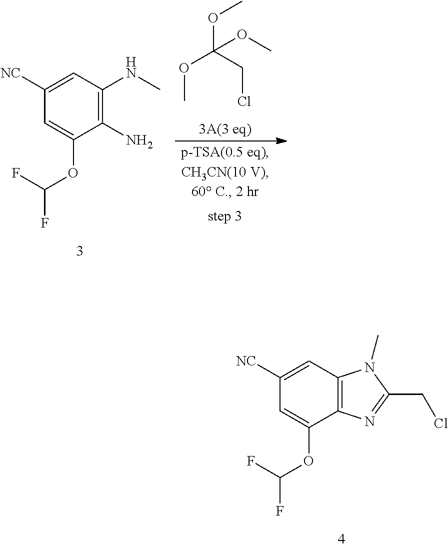

To a solution of 3 (2 g, 9.38 mmol, 1 eq) in CH₃CN (20 mL) was added 3A (4.35 g, 28.14 mmol, 3.79 mL, 3 eq) and p-TSA (892.27 mg, 4.69 mmol, 0.5 eq). The mixture was stirred at 60° C. for 2 hr. LCMS (product: RT=0.445 min) showed the 3 was consumed completely. The reaction mixture was concentrated under reduced pressure to remove solvent. The residue was diluted with H₂O 30 mL and extracted with EtOAc 20 mL*3. The combined organic layers were washed with brine 10 mL*2, dried over Na₂SO₄, filtered and concentrated under reduced pressure to give a residue. 4 (3 g, crude) was obtained as a black solid.

### General Procedure for Preparation of Intermediate 5:

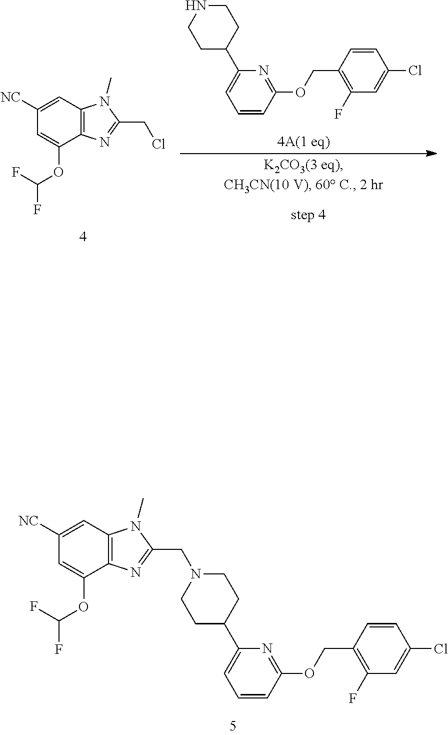 [SMILES: Cn1c(CCl)nc2c(OC(F)F)cc(C#N)cc21 ; Fc1cc(Cl)ccc1COc1cccc(C2CCNCC2)n1 ; Cn1c(CN2CCC(c3cccc(OCc4ccc(Cl)cc4F)n3)CC2)nc2c(OC(F)F)cc(C#N)cc21]

A mixture of 4 (2 g, 6.23 mmol, 1 eq), 4A (1.69 g, 6.23 mmol, 1 eq), K₂CO₃ (2.58 g, 18.70 mmol, 3 eq) in CH₃CN (20 mL) was degassed and purged with N₂ for 3 times, and then the mixture was stirred at 60° C. for 2 hr under N₂ atmosphere. LCMS (ET64759-562-P1A, product: RT=0.527 min) showed the 4 was consumed completely. The reaction mixture was quenched by addition H₂O 40 mL at 20° C. and extracted with EtOAc 30 mL*3. The combined organic layers were washed with brine 20 mL*2, dried over Na₂SO₄, filtered, and concentrated under reduced pressure to give a residue. The residue was purified by column chromatography (SiO₂, Petroleum ether/Ethyl acetate=5/1 to 1/1). 5 (3 g, crude) was obtained as a yellow oil.

¹H NMR (400 MHz, CHCl3-d) δ=7.56 (d, J=1.2 Hz, 1H), 7.54-7.48 (m, 1H), 7.44 (t, J=8.4 Hz, 1H), 7.40 (t, J=73.6 Hz, 1H), 7.29 (s, 1H), 7.16-7.08 (m, 2H), 6.78-6.71 (m, 1H), 6.62 (d, J=8.4 Hz, 1H), 5.41 (s, 2H), 4.01 (s, 3H), 3.96-3.85 (m, 2H), 3.06-2.86 (m, 2H), 2.70-2.57 (m, 1H), 2.43-2.20 (m, 2H), 2.00-1.73 (m, 4H)

### General Procedure for Preparation of Intermediate 6:

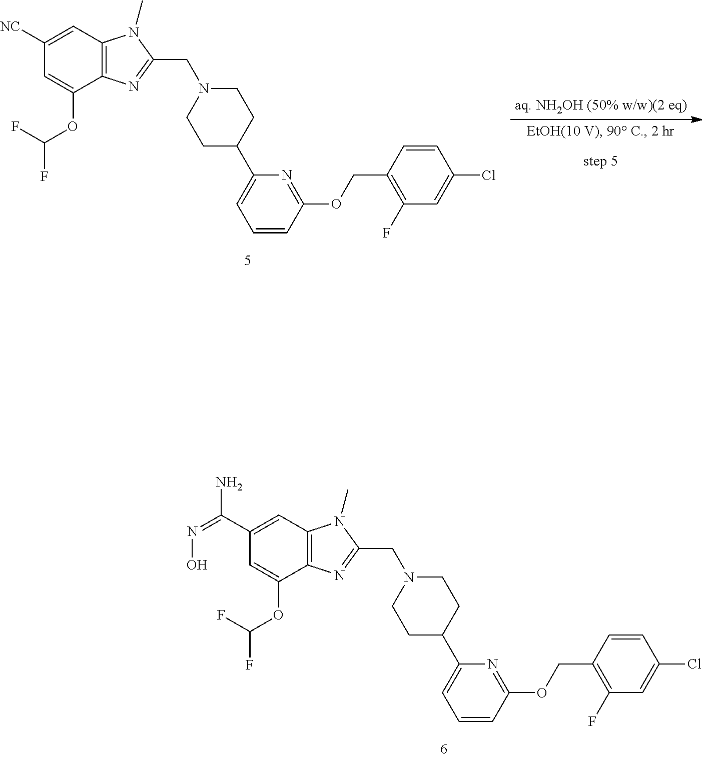 [SMILES: Cn1c(CN2CCC(c3cccc(OCc4ccc(Cl)cc4F)n3)CC2)nc2c(OC(F)F)cc(C#N)cc21 ; Cn1c(CN2CCC(c3cccc(OCc4ccc(Cl)cc4F)n3)CC2)nc2c(OC(F)F)cc(/C(N)=N\O)cc21]

A mixture of 5 (500 mg, 899.32 μmol, 1 eq), aq. NH₂OH (50% w/w) (118.82 mg, 1.80 mmol, 50% purity, 2 eq) in EtOH (5 mL) was degassed and purged with N₂ for 3 times, and then the mixture was stirred at 90° C. for 2 hr under N₂ atmosphere. LCMS (product: RT=1.446 min) showed the 5 was consumed completely. The reaction mixture was quenched by addition H₂O 10 mL at 20° C. and extracted with EtOAc 20 mL*3. The combined organic layers were washed with brine 10 mL*2, dried over Na₂SO₄, filtered, and concentrated under reduced pressure to give a residue. The residue was triturated with MTBE at 20° C. for 30 min. The solution was filtered, the solid under reduced pressure to give product. 6 (770 mg, crude) was obtained as a white solid.

¹H NMR (400 MHz, DMSO-d6) δ=9.67 (s, 1H), 7.78 (d, J=1.2 Hz, 1H), 7.65-7.59 (m, 2H), 7.61 (t, J=74.8 Hz, 1H) 7.55 (t, J=8.4 Hz, 1H), 7.46 (d, J=2.0 Hz, 1H), 7.43 (d, J=2.0 Hz, 1H), 7.34 (s, 1H), 7.28 (dd, J=2.0, 8.4 Hz, 1H), 6.86 (d, J=7.2 Hz, 1H), 6.67 (d, J=8.4 Hz, 1H), 5.90 (s, 2H), 5.36 (s, 2H), 3.91 (s, 3H), 3.82 (s, 2H), 2.93 (d, J=11.2 Hz, 2H), 2.63-2.54 (m, 1H), 2.20 (t, J=10.8 Hz, 2H), 1.82-1.64 (m, 4H)

### General Procedure for Preparation of Compound 236 (7):

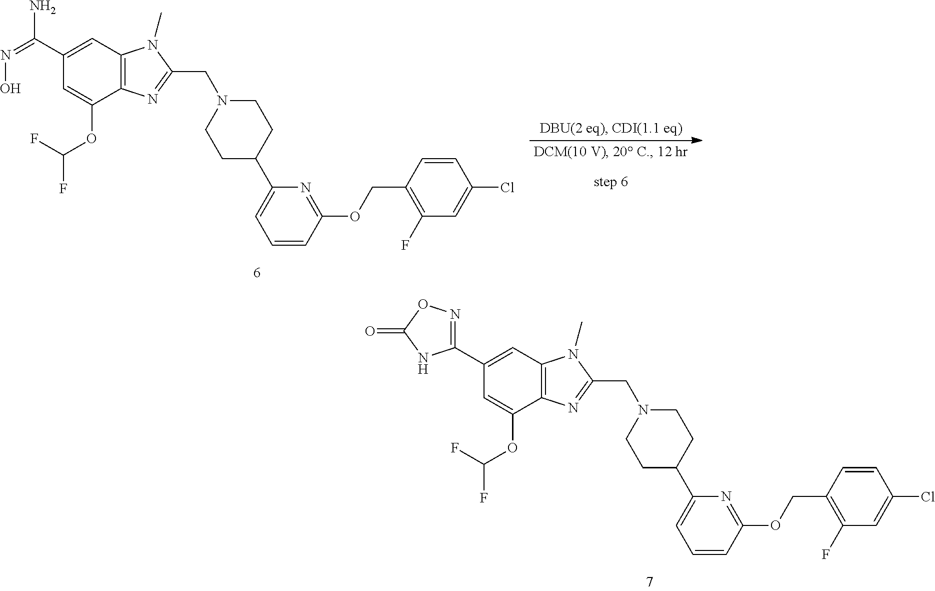 [SMILES: Cn1c(CN2CCC(c3cccc(OCc4ccc(Cl)cc4F)n3)CC2)nc2c(OC(F)F)cc(/C(N)=N\O)cc21 ; Cn1c(CN2CCC(c3cccc(OCc4ccc(Cl)cc4F)n3)CC2)nc2c(OC(F)F)cc(-c3noc(=O)[nH]3)cc21]

A mixture of 6 (100 mg, 169.78 μmol, 1 eq), DBU (51.69 mg, 339.55 μmol, 51.18 μL, 2 eq) in DCM (1 mL) was added CDI (30.28 mg, 186.75 μmol, 1.1 eq), then degassed and purged with N₂ for 3 times, and then the mixture was stirred at 20° C. for 12 hr under N₂ atmosphere. LCMS (product: RT=1.308 min) showed 6 was completely. The reaction mixture was filtered, and the filtrate was purified by prep-HPLC (column: Waters Xbridge Prep OBD C18 150*40 mm*10 um; mobile phase: [H₂O (10 mM NH₄HCO₃)—CH₃CN]; gradient: 30%-60% B over 8.0 min). 3-(2-((4-(6-((4-Chloro-2-fluorobenzyl)oxy)pyridin-2-yl)piperidin-1-yl)methyl)-4-(difluoromethoxy)-1-methyl-1H-benzo[d]imidazol-6-yl)-1,2,4-oxadiazol-5 (4H)-one (30.28 mg, 49.24 μmol, 29.00% yield, 100% purity) was obtained as a white solid.

LCMS: RT=2.923 min, MS cal.: 614.2, [M+H]⁺=615.2

HPLC: RT=7.757 min

¹H NMR (400 MHz, MeOH-d4) δ=7.87 (d, J=1.2 Hz, 1H), 7.61-7.55 (m, 1H), 7.51-7.46 (m, 2H), 7.34 (t, J=74 Hz, 1H), 7.22-7.16 (m, 2H), 6.83 (d, J=7.2 Hz, 1H), 6.64 (d, J=8.4 Hz, 1H), 5.40 (s, 2H), 4.04 (s, 2H), 4.02 (s, 3H), 3.15 (d, J=11.6 Hz, 2H), 2.75-2.65 (m, 1H), 2.53-2.42 (m, 2H), 1.97-1.87 (m, 4H)

### Example A157—Preparation of Compound 238. 4-(Difluoromethoxy)-2-((4-(6-((2-fluoro-4-methylbenzyl)oxy)pyridin-2-yl)piperidin-1-yl)methyl)-1-methyl-1H-benzo[d]imidazole-6-carboxylic acid (Compound 238) (3)

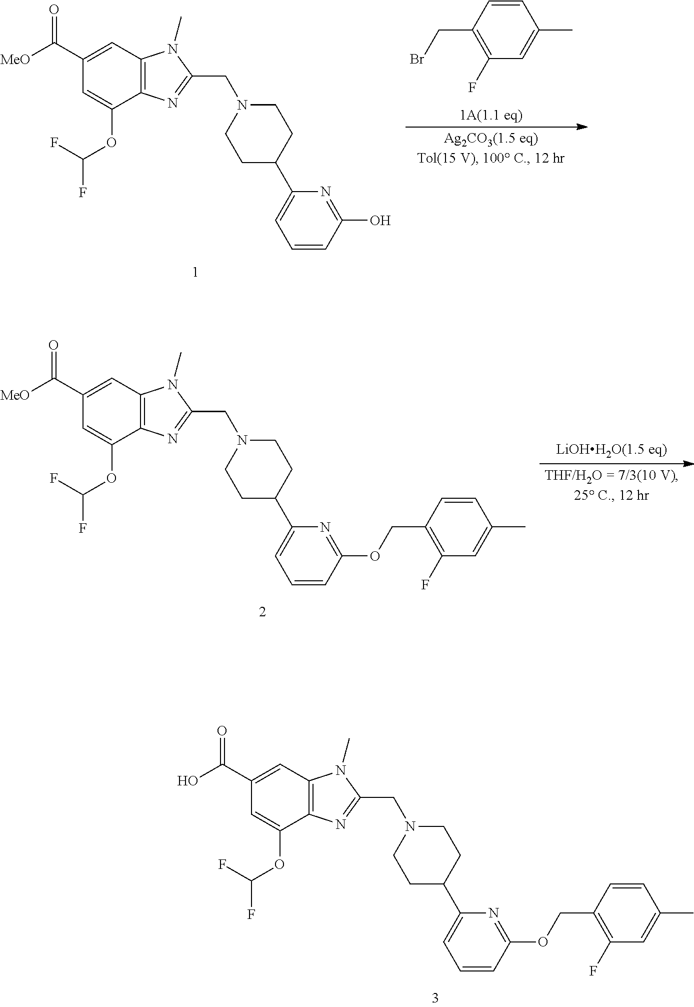

### General Procedure for Preparation of Intermediate 2:

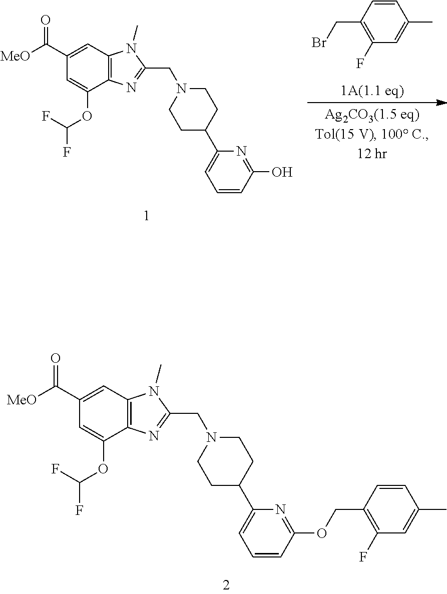

A mixture of 1 (200 mg, 447.98 μmol, 1 eq), 1A (100.06 mg, 492.78 μmol, 1.1 eq), Ag₂CO₃ (185.29 mg, 671.97 μmol, 30.49 μL, 1.5 eq) in Toluene (3 mL) was degassed and purged with N₂ for 3 times, and then the mixture was stirred at 100° C. for 12 hr under N₂ atmosphere. LC-MS (product, Rt=1.677 min) showed 1 was consumed completely and desired mass was detected. The suspension was filtered through a pad of Celite, filter cake was washed with EtOAc (10 mL*3). The reaction mixture was diluted with H₂O 15 mL and extracted with EtOAc 60 mL (20 mL*3). The combined organic layers were washed with brine 20 mL, dried over Na₂SO₄, filtered and concentrated under reduced pressure to give a residue. The residue was purified by column chromatography (SiO₂, Petroleum ether/Ethyl acetate=10/1 to 1/1). 2 (220 mg, 386.93 μmol, 86.37% yield) was obtained as a white solid.

LCMS: RT=1.677 min, MS cal.: 568.23, [M+1]⁺=569.0

¹H NMR (400 MHz, DMSO) δ=8.14-8.09 (m, 1H), 7.65 (t, J=74.4 Hz 1H), 7.60 (t, J=7.2 Hz, 1H), 7.53 (s, 1H), 7.41 (t, J=7.2 Hz, 1H), 7.07-6.95 (m, 2H), 6.85 (d, J=7.2 Hz, 1H), 6.63 (d, J=8.0 Hz, 1H), 5.32 (s, 2H), 3.98 (s, 3H), 3.90 (s, 3H), 3.86 (s, 2H), 2.95 (br d, J=11.2 Hz, 2H), 2.62-2.55 (m, 1H), 2.29 (s, 3H), 2.26-2.16 (m, 2H), 1.87-1.85 (m, 1H), 1.87-1.66 (m, 3H)

### General Procedure for Preparation of Compound 238 (3):

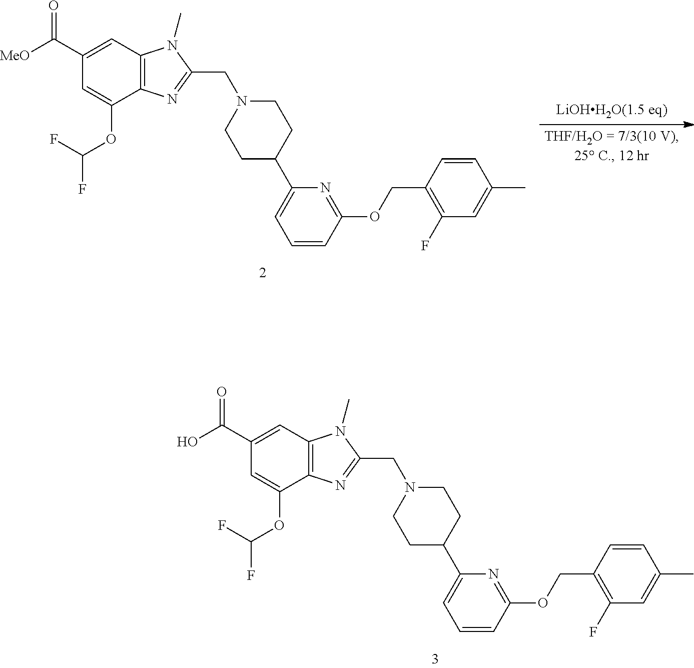 [SMILES: COC(=O)c1cc(OC(F)F)c2nc(CN3CCC(c4cccc(OCc5ccc(C)cc5F)n4)CC3)n(C)c2c1 ; Cc1ccc(COc2cccc(C3CCN(Cc4nc5c(OC(F)F)cc(C(=O)O)cc5n4C)CC3)n2)c(F)c1]

A mixture of 2 (100 mg, 175.88 μmol, 1 eq), LiOH·H₂O (11.07 mg, 263.81 μmol, 1.5 eq) in THF (1.4 mL) and H₂O (0.6 mL) was degassed and purged with N₂ for 3 times, and then the mixture was stirred at 25° C. for 12 hr under N₂ atmosphere. LC-MS (product, Rt=2.443 min) showed 2 was consumed completely and one main peak with desired m/z was detected. The reaction was directly purified. The residue was purified by prep-HPLC (column: Waters Xbridge Prep OBD C18 150*40 mm*10 um; mobile phase: [H₂O (10 mM NH₄HCO₃)-ACN]; gradient: 20%-70% B over 8.0 min) 4-(Difluoromethoxy)-2-((4-(6-((2-fluoro-4-methylbenzyl)oxy)pyridin-2-yl)piperidin-1-yl)methyl)-1-methyl-1H-benzo[d]imidazole-6-carboxylic acid (29.88 mg, 53.84 μmol, 30.61% yield, 99.93% purity) was obtained as a white solid.

LCMS: RT=2.443 min, MS cal.: 554.21, [M+H]⁺=555.2

HPLC: RT=11.88 min, purity: 99.93%

¹H NMR (400 MHz, MEOH-D4) δ=8.10 (s, 1H), 7.68 (s, 1H), 7.56 (t, J=7.2 Hz, 1H), 7.35 (t, J=7.2 Hz, 1H), 7.25 (t, J=74.4 Hz, 1H), 6.97-6.89 (m, 2H), 6.81 (d, J=7.2 Hz, 1H), 6.61 (d, J=8.4 Hz, 1H), 5.36 (s, 2H), 4.02 (s, 3H), 4.01 (s, 2H), 3.12 (br d, J=11.2 Hz, 2H), 2.76-2.60 (m, 1H), 2.50-2.38 (m, 2H), 2.31 (s, 3H), 1.99-1.83 (m, 4H)

### Example A158—Preparation of Compound 239. 2-((4-(2-((4-Chloro-2-fluorobenzyl)amino)pyridin-3-yl)piperidin-1-yl)methyl)-4-(difluoromethoxy)-1-methyl-1H-benzo[d]imidazole-6-carboxylic acid (Compound 239) (7)

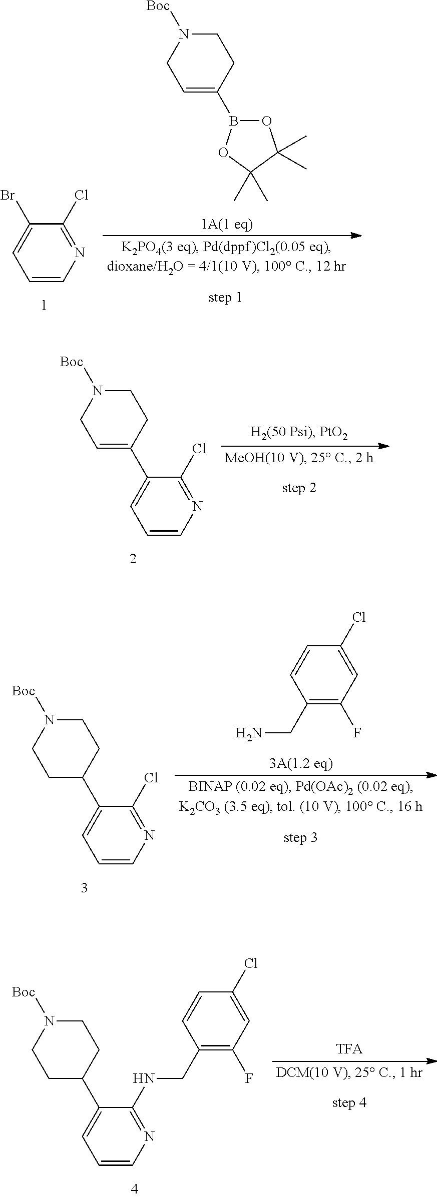 [SMILES: Clc1ncccc1Br ; CC(C)(C)OC(=O)N1CC=C(B2OC(C)(C)C(C)(C)O2)CC1 ; CC(C)(C)OC(=O)N1CC=C(c2cccnc2Cl)CC1 ; CC(C)(C)OC(=O)N1CCC(c2cccnc2Cl)CC1 ; NCc1ccc(Cl)cc1F ; CC(C)(C)OC(=O)N1CCC(c2cccnc2NCc2ccc(Cl)cc2F)CC1]

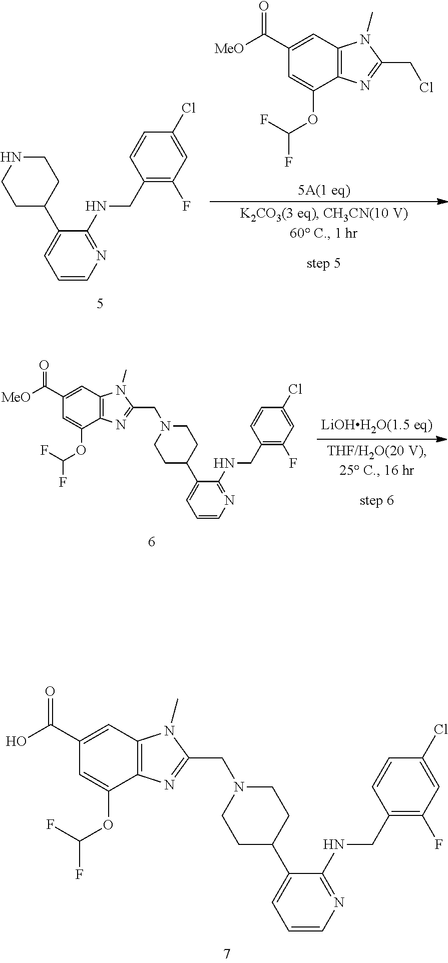 [SMILES: Fc1cc(Cl)ccc1CNc1ncccc1C1CCNCC1 ; COC(=O)c1cc(OC(F)F)c2nc(CCl)n(C)c2c1 ; COC(=O)c1cc(OC(F)F)c2nc(CN3CCC(c4cccnc4NCc4ccc(Cl)cc4F)CC3)n(C)c2c1 ; Cn1c(CN2CCC(c3cccnc3NCc3ccc(Cl)cc3F)CC2)nc2c(OC(F)F)cc(C(=O)O)cc21]

### General Procedure for Preparation of Intermediate 2:

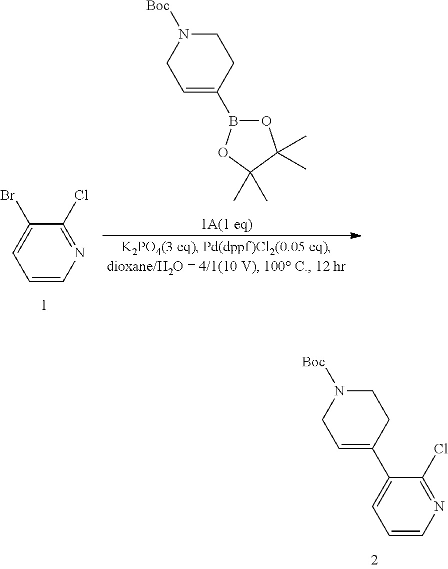 [SMILES: Clc1ncccc1Br ; CC(C)(C)OC(=O)N1CC=C(B2OC(C)(C)C(C)(C)O2)CC1 ; CC(C)(C)OC(=O)N1CC=C(c2cccnc2Cl)CC1]

To a solution of 1 (3 g, 15.59 mmol, 1 eq) and 1A (4.82 g, 15.59 mmol, 1 eq) in dioxane (60 mL) and H₂O (15 mL) was added K₃PO₄ (9.93 g, 46.77 mmol, 3 eq) and Pd(dppf)Cl₂ (570.33 mg, 779.46 μmol, 0.05 eq). The mixture was stirred at 100° C. for 12 hr. TLC indicated 1 was consumed completely and one new spot formed. The reaction was clean according to TLC. The mixture was filtered by celite pad and filtrate was added water (200 mL), extracted with EtOAc (200 mL*2), the organic phase was washed with brine (150 mL), filtered, and concentrated in vacuum to get a residue. The residue was purified by column chromatography (SiO₂, Petroleum ether/Ethyl acetate=1/0 to 10/1). 2 (4.1 g, 13.91 mmol, 89.22% yield) was obtained as a yellow oil. Checked by HNMR (ET78548-44-P1B1).

1H NMR (400 MHz, CHCl3-d) δ=8.34-8.29 (m, 1H) 7.51 (dd, J=7.2, 1.18 Hz, 1H) 7.25-7.19 (m, 1H) 5.73 (br s, 1H) 4.07 (br s, 2H) 3.64 (br t, J=5.2 Hz, 2H) 2.50-2.41 (m, 2H) 1.50 (s, 9H)

### General Procedure for Preparation of Intermediate 3:

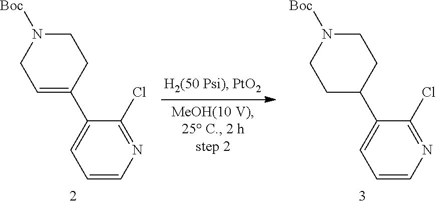

MeOH (15 mL) was charged to the 75 mL Hydrogenated bottle flask, then PtO₂ (800 mg) and 2 (1.5 g, 5.09 mmol, 1 eq) was added at 25° C. After the addition, purged with H₂ for 3 times the mixture was stirred at 25° C. 50 psi for 2 hr. TLC indicated 2 was consumed completely and one new spot formed. The reaction was clean according to TLC. The suspension was filtered through diatomite and filter cake was washed with EtOAc 10 ml*3. The combined organic layers were reduced pressure to give a residue. The residue was purified by column chromatography (SiO₂, Petroleum ether/Ethyl acetate=1/0 to 10/1). 3 (1 g, 3.37 mmol, 66.21% yield) was obtained as a white oil.

LCMS: Rt₁=0.548 min, MS cal.: 296.1, [M+H]⁺=297.1

1H NMR (400 MHz, MeOH-d4) δ=8.22 (dd, J=4.8, 1.79 Hz, 1H) 7.81 (dd, J=7.6, 1.85 Hz, 1H) 7.38 (dd, J=7.2, 4.77 Hz, 1H) 4.25 (br d, J=13.2 Hz, 2H) 4.09 (br d, J=13.24 Hz, 1H) 3.19 (tt, J=12.0, 3.35 Hz, 1H) 2.91 (br s, 2H) 2.69 (br s, 1H) 1.88 (br d, J=13.2 Hz, 2H) 1.71 (br d, J=12.0 Hz, 1H) 1.59 (qd, J=12.4, 4.05 Hz, 2H) 1.48 (s, 10H) 1.45 (s, 4H) 1.35-1.21 (m, 1H) 1.18-1.02 (m, 1H)

### General Procedure for Preparation of Intermediate 4:

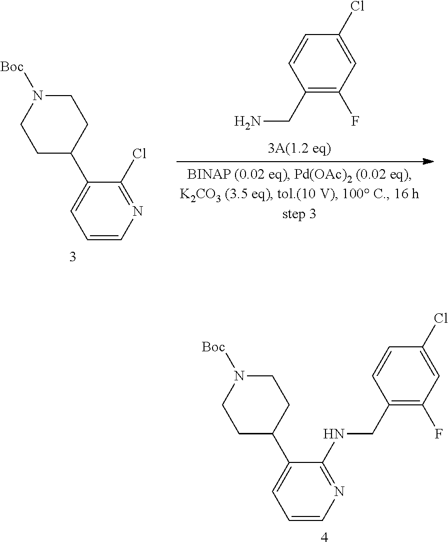

A mixture of 3 (500 mg, 1.68 mmol, 1 eq), 3A (537.71 mg, 3.37 mmol, 2 eq), BINAP (20.98 mg, 33.69 μmol, 0.02 eq), K₂CO₃ (814.91 mg, 5.90 mmol, 3.5 eq) and Pd(OAc)₂ (7.56 mg, 33.69 μmol, 0.02 eq) in toluene (5 mL) was degassed and purged with N₂ for 3 times, and then the mixture was stirred at 100° C. for 16 hr under N₂ atmosphere. LC-MS (Rt=1.553 min) showed 3 was consumed completely and one main peak with desired mass was detected. The reaction mixture was diluted with H₂O 20 mL and extracted with EtOAc 60 mL (20 mL*3). The combined organic layers were washed with brine 20 mL, dried over Na₂SO₄, filtered, and concentrated under reduced pressure to give a residue. The residue was purified by prep-HPLC (TFA condition column: Phenomenex luna C18 100*40 mm*5 um; mobile phase: [H₂O (0.2% FA)-ACN]; gradient: 25%-60% B over 8.0 min). 4 (210 mg, 500.10 μmol, 29.68% yield) was obtained as a yellow solid.

LCMS: Rt=1.553 min, MS cal.: 419.1, [M+H]⁺=420.1

1H NMR (400 MHz, MeOH-d4) δ=7.80 (dd, J=5.2, 1.67 Hz, 1H) 7.37 (dd, J=7.4, 1.43 Hz, 1H) 7.24 (t, J=8.4 Hz, 1H) 7.15 (dd, J=10.0, 1.97 Hz, 1H) 7.09 (dd, J=8.4, 1.73 Hz, 1H) 6.60 (dd, J=7.4, 5.13 Hz, 1H) 4.66 (s, 2H) 4.22 (br d, J=13.2 Hz, 2H) 2.93 (br s, 2H) 2.78 (tt, J=12.0, 3.07 Hz, 1H) 1.92-1.83 (m, 2H) 1.58-1.50 (m, 2H) 1.48 (s, 9H)

### General Procedure for Preparation of Intermediate 5:

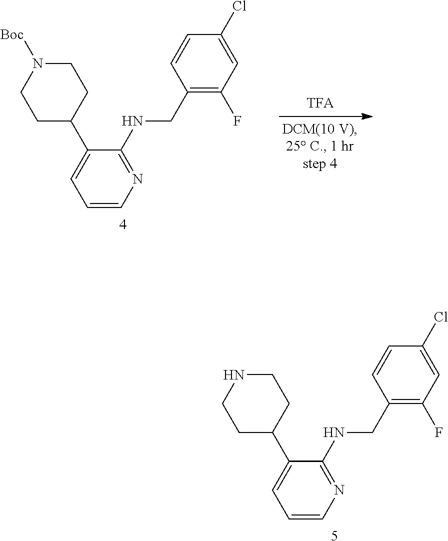

A mixture of Compound 3 (100 mg, 238.14 μmol, 1 eq), TFA (307.00 mg, 2.69 mmol, 0.2 mL, 11.31 eq), in DCM (1 mL) was degassed and purged with N₂ for 3 times, and then the mixture was stirred at 25° C. for 1 hr under N₂ atmosphere. LC-MS (product Rt=0.27-86 min) showed Reactant 1 was consumed completely and one main peak with desired mass was detected. The reaction mixture was concentrated under reduced pressure to remove solvent. 4 (103 mg, crude, TFA) was obtained as a white solid.

LCMS: Rt₁=0.286 min, MS cal.: 319.1, [M+H]⁺=320.0

### General Procedure for Preparation of Intermediate 6:

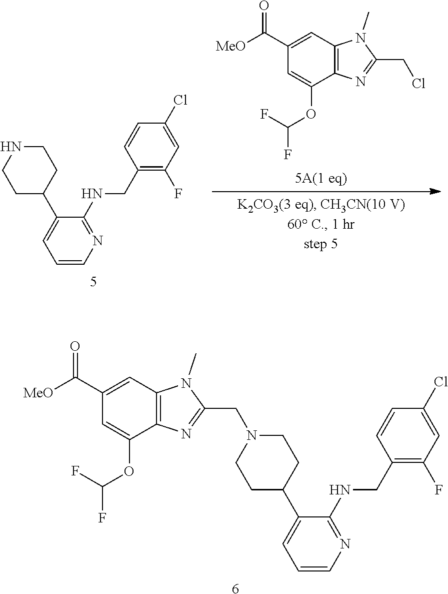

A mixture of 5 (103 mg, 237.42 μmol, 1 eq, TFA), 5A (72.34 mg, 237.42 μmol, 1 eq), K₂CO₃ (98.44 mg, 712.27 μmol, 3 eq), in CH₃CN (1 mL) was degassed and purged with N₂ for 3 times, and then the mixture was stirred at 60° C. for 1 hr under N₂ atmosphere. LC-MS (product Rt=1.537 min) showed 5 was consumed completely and one main peak with desired mass was detected. The reaction mixture was diluted with H₂O 15 mL and extracted with EtOAc 45 mL (15 mL*3). The combined organic layers were washed with brine 15 mL, dried over Na₂SO₄, filtered and concentrated under reduced pressure to give a residue. The residue was purified by column chromatography (SiO₂, Petroleum ether/Ethyl acetate=100/1 to 1/1). Compound 6 (90 mg, 153.06 μmol, 64.47% yield) was obtained as a white solid.

LCMS: Rt=1.537 min, MS cal.: 587.1, [M+H]⁺=588.1

### General Procedure for Preparation of Compound 239 (7):

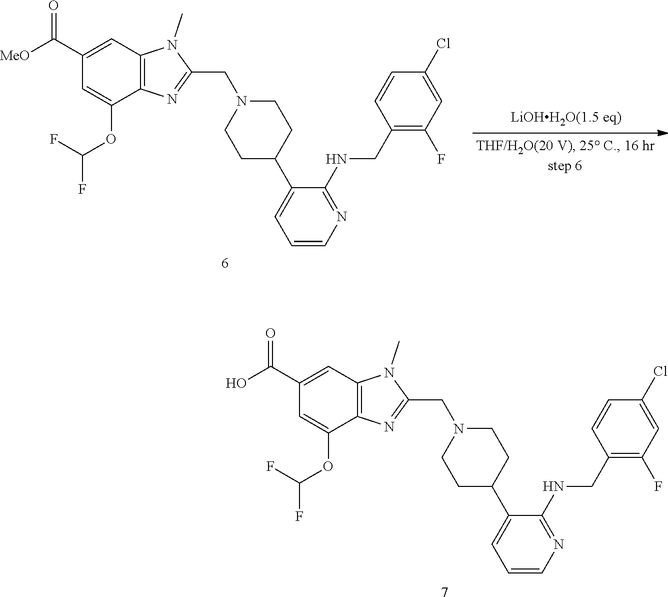 [SMILES: COC(=O)c1cc(OC(F)F)c2nc(CN3CCC(c4cccnc4NCc4ccc(Cl)cc4F)CC3)n(C)c2c1 ; "6" ; Cn1c(CN2CCC(c3cccnc3NCc3ccc(Cl)cc3F)CC2)nc2c(OC(F)F)cc(C(=O)O)cc21 ; "7"]

A mixture of Compound 6 (80 mg, 136.05 μmol, 1 eq), LiOH·H₂O (8.56 mg, 204.07 μmol, 1.5 eq), in H₂O (0.3 mL) THF (0.7 mL) was degassed and purged with N₂ for 3 times, and then the mixture was stirred at 25° C. for 16 hr under N₂ atmosphere. TLC (Petroleum ether:Ethyl acetate=0:1, Rf=0.10) indicated Compound 6 was consumed completely and one new spot formed. The reaction was clean according to TLC. The residue was purified by prep-HPLC (neutral condition column: Waters Xbridge Prep OBD C18 150*40 mm*10 um; mobile phase: [H₂O (10 mM NH₄HCO₃)-ACN]; gradient: 25%-75% B over 8.0 min). 2-((4-(2-((4-Chloro-2-fluorobenzyl)amino)pyridin-3-yl)piperidin-1-yl)methyl)-4-(difluoromethoxy)-1-methyl-1H-benzo[d]imidazole-6-carboxylic acid (27.84 mg, 48.50 μmol, 35.65% yield) was obtained as a white solid.

LCMS: Rt₁=2.597 min, MS cal.: 573.1, [M+H]⁺=574.2

1H NMR (400 MHz, DMSO-d6) δ=8.06 (s, 1H) 7.78 (dd, J=4.8, 1.37 Hz, 1H) 7.63 (t, J=74.4 Hz, 1H) 7.52 (s, 1H) 7.35 (dd, J=10.4, 1.97 Hz, 1H) 7.30 (dd, J=7.2, 1.19 Hz, 1H) 7.27-7.21 (m, 1H) 7.19-7.14 (m, 1H) 6.72 (t, J=5.2 Hz, 1H) 6.51 (dd, J=7.2, 5.01 Hz, 1H) 4.56 (br d, J=5.2 Hz, 2H) 3.96 (s, 3H) 3.88 (s, 2H) 2.96 (br d, J=12.0 Hz, 2H) 2.64 (br t, J=11.2 Hz, 1H) 2.31 (br t, J=10.8 Hz, 2H) 1.80 (br d, J=11.2 Hz, 2H) 1.63-1.48 (m, 2H)

### Example A159—Preparation of Compound 242. 2-((4-(6-((4-Chloro-2-fluorobenzyl)oxy)-5-fluoropyridin-2-yl)piperidin-1-yl)methyl)-4-(difluoromethoxy)-1-methyl-1H-benzo[d]imidazole-6-carboxylic acid (Compound 242) (5)

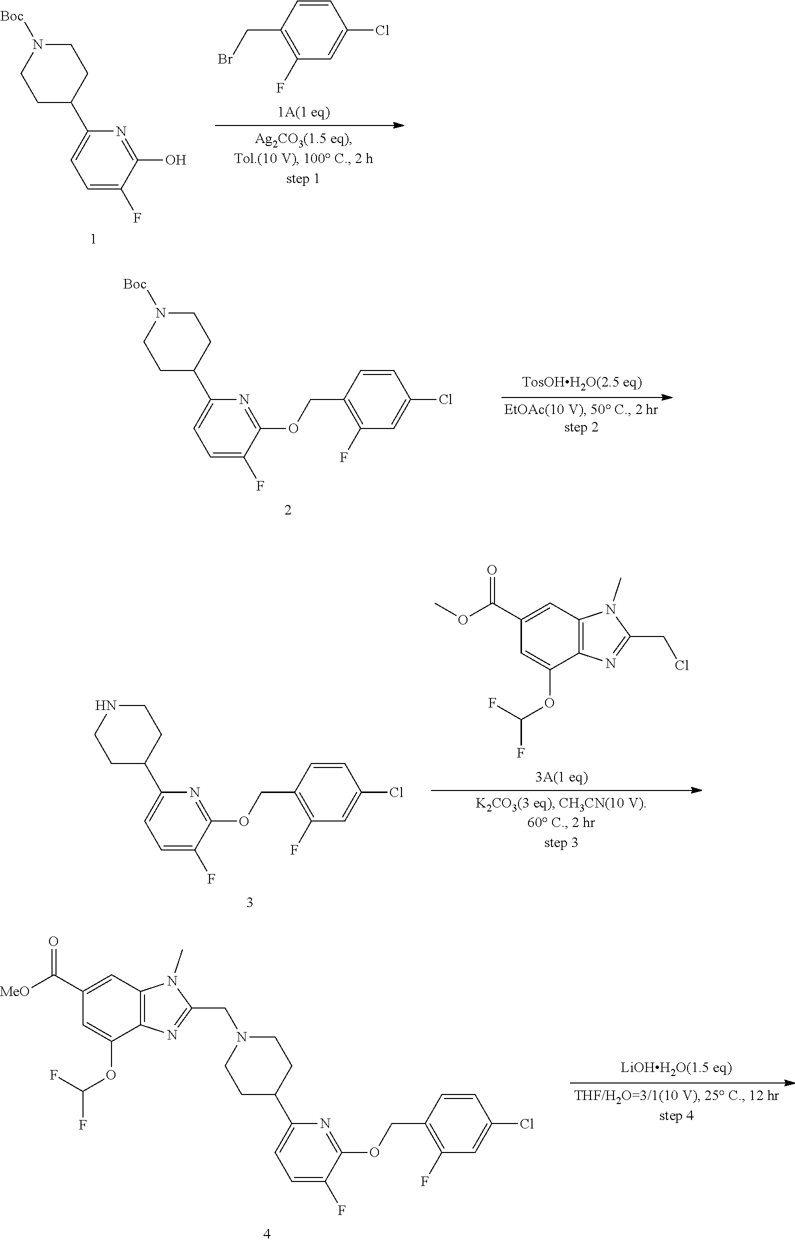

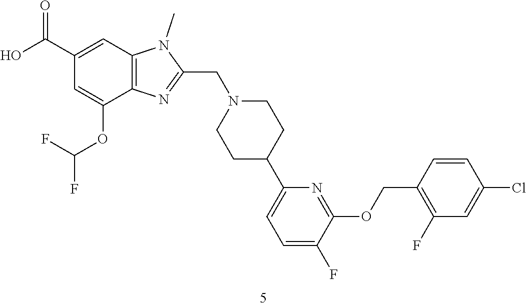 [SMILES: Cn1c(CN2CCC(c3ccc(F)c(OCc4ccc(Cl)cc4F)n3)CC2)nc2c(OC(F)F)cc(C(=O)O)cc21]

### General Procedure for Preparation of Intermediate 2:

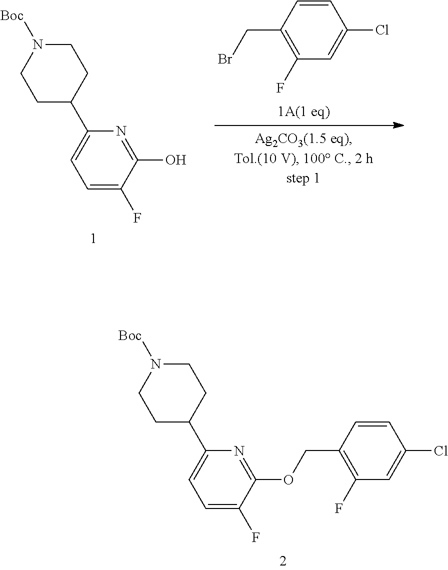 [SMILES: CC(C)(C)OC(=O)N1CCC(c2ccc(F)c(O)n2)CC1 ; Fc1cc(Cl)ccc1CBr ; "1" ; CC(C)(C)OC(=O)N1CCC(c2ccc(F)c(OCc3ccc(Cl)cc3F)n2)CC1]

To a solution of 1 (100 mg, 337.45 μmol, 1 eq), 1A (75.41 mg, 337.45 μmol, 1 eq) in toluene (2 mL) was added Ag₂CO₃ (139.58 mg, 506.18 μmol, 22.97 μL, 1.5 eq). The mixture was stirred at 100° C. for 2 hr. LC-MS showed 1 was consumed completely and desired mass was detected. Two batches of crude product mixtures were combined for workup. The residue was diluted with H₂O (10 mL) and extracted with EtOAc 45 mL (15 mL*3). The combined organic layers were washed with brine (10 mL), dried over Na₂SO₄ filtered and concentrated under reduced pressure to give a residue. The residue was purified by column chromatography (SiO₂, Petroleum ether/Ethyl acetate=100/1 to 10/1). 2 (230 mg, 524.04 μmol, 77.65% yield) was obtained as a colorless oil.

LCMS: RT=1.723 min, MS cal.: 438.2, 440.1, [M+H]⁻=436.9

¹H NMR (400 MHz, DMSO-d₆) δ=7.64-7.51 (m, 2H), 7.49-7.42 (m, 1H), 7.31 (dd, J=1.6, 8.4 Hz, 1H), 6.90 (dd, J=2.8, 8.0 Hz, 1H), 5.45 (s, 2H), 4.11-3.95 (m, 2H), 2.90-2.71 (m, 3H), 1.79-1.71 (m, 2H), 1.60-1.46 (m, 2H), 1.41 (s, 9H)

### General Procedure for Preparation of Intermediate 3:

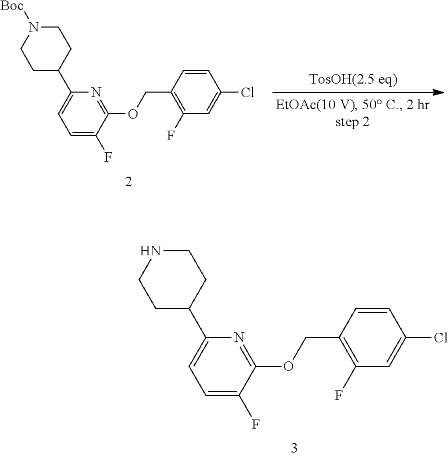 [SMILES: CC(C)(C)OC(=O)N1CCC(c2ccc(F)c(OCc3ccc(Cl)cc3F)n2)CC1 ; Fc1cc(Cl)ccc1COc1nc(C2CCNCC2)ccc1F]

To a solution of 2 (100 mg, 227.85 μmol, 1 eq) in EtOAc (1 mL) was added TosOH (98.09 mg, 569.61 μmol, 2.5 eq). The mixture was stirred at 50° C. for 2 hr. TLC (PE:EtOAc=5:1) showed the 2 was consumed completely. The mixture was added aq. Na₂CO₃ to adjust pH=7-8. The mixture was diluted with H₂O (20 mL) and extracted with DCM 30 mL (10 mL*3). The combined organic layers were washed with brine (20 mL), dried over Na₂SO₄, filtered, and concentrated under reduced pressure to give a residue. 3 (70 mg, 206.62 μmol, 90.69% yield) was obtained as a yellow oil.

LCMS: RT=0.429 min, MS cal.: 338.1, 338.2, [M+H]⁺=339.0

¹H NMR (400 MHz, MeOH-d4) δ=7.53 (t, J=8.4 Hz, 1H), 7.38 (dd, J=8.0, 10.4 Hz, 1H), 7.27-7.17 (m, 2H), 6.81 (dd, J=2.8, 8.0 Hz, 1H), 5.50 (s, 2H), 3.18-3.08 (m, 2H), 2.80-2.66 (m, 3H), 1.90-1.81 (m, 2H), 1.80-1.65 (m, 2H)

### General Procedure for Preparation of Intermediate 4:

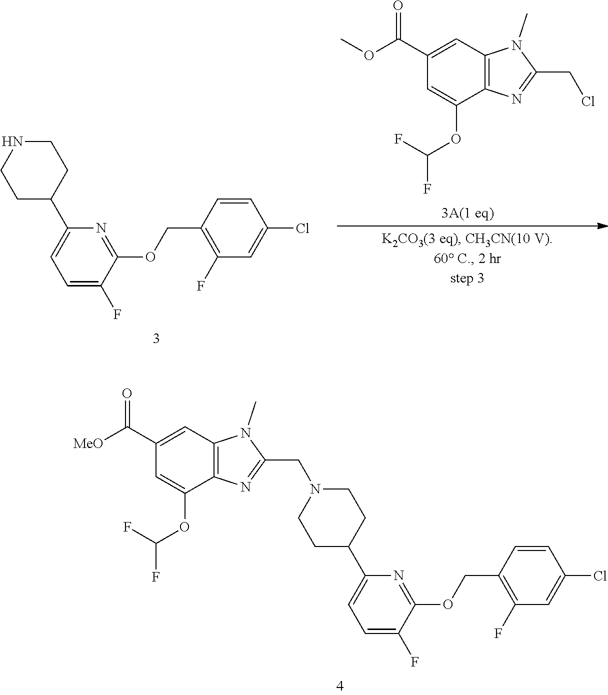 [SMILES: Fc1cc(Cl)ccc1COc1nc(C2CCNCC2)ccc1F ; COC(=O)c1cc(OC(F)F)c2nc(CCl)n(C)c2c1 ; COC(=O)c1cc(OC(F)F)c2nc(CN3CCC(c4ccc(F)c(OCc5ccc(Cl)cc5F)n4)CC3)n(C)c2c1]

To a solution of 3 (60 mg, 177.11 μmol, 1 eq), 3A (53.96 mg, 177.11 μmol, 1 eq) in CH₃CN (1 mL) was added K₂CO₃ (73.43 mg, 531.32 μmol, 3 eq). The mixture was stirred at 60° C. for 2 hr. LC-MS showed 3 was consumed completely and desired mass was detected. The reaction mixture was diluted with H₂O (20 mL) and extracted with EtOAc (10 mL*3). The combined organic layers were washed with brine (20 mL), dried over Na₂SO₄, filtered, and concentrated under reduced pressure to give a residue. The residue was purified by column chromatography (SiO₂, Petroleum ether/Ethyl acetate=2/1 to 0/1). 4 (90 mg, 148.27 μmol, 83.72% yield) was obtained as a yellow oil.

LCMS: RT=0.983 min, MS cal.: 606.1, [M+H]⁺=607.1

¹H NMR (400 MHz, DMSO-d₆) δ=8.14-8.09 (m, 1H), 7.65 (t, J=74.4 Hz, 1H), 7.62-7.51 (m, 3H), 7.47 (br d, J=7.6 Hz, 1H), 7.30 (br d, J=8.0 Hz, 1H), 6.93-6.85 (m, 1H), 5.44 (s, 2H), 4.01-3.95 (m, 3H), 3.93-3.80 (m, 5H), 2.93 (br d, J=10.4 Hz, 2H), 2.54 (br s, 1H), 2.27-2.14 (m, 2H), 1.83-1.74 (m, 2H), 1.74-1.59 (m, 3H)

### General Procedure for Preparation of Compound 242 (5):

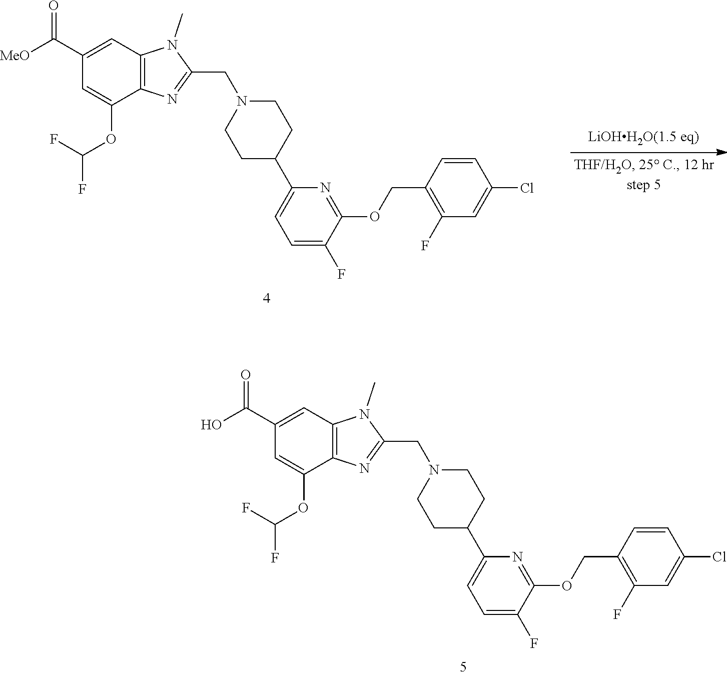 [SMILES: COC(=O)c1cc(OC(F)F)c2nc(CN3CCC(c4ccc(F)c(OCc5ccc(Cl)cc5F)n4)CC3)n(C)c2c1 ; Cn1c(CN2CCC(c3ccc(F)c(OCc4ccc(Cl)cc4F)n3)CC2)nc2c(OC(F)F)cc(C(=O)O)cc21]

To a solution of 4 (80 mg, 131.80 μmol, 1 eq) in THF (1 mL) was added LiOH H₂O (8.30 mg, 197.70 μmol, 1.5 eq) in H₂O (0.3 mL). The mixture was stirred at 25° C. for 12 hr. LC-MS showed 4 was consumed completely and desired mass was detected. The reaction mixture was purified directly. The residue was purified by prep-HPLC (neutral condition; column: Waters Xbridge Prep OBD C18 150*40 mm*10 um; mobile phase: [H₂O (10 mM NH₄HCO₃)-ACN]; gradient: 20%-70% B over 8.0 min). 2-((4-(6-((4-Chloro-2-fluorobenzyl)oxy)-5-fluoropyridin-2-yl)piperidin-1-yl)methyl)-4-(difluoromethoxy)-1-methyl-1H-benzo[d]imidazole-6-carboxylic acid (28.16 mg, 47.49 μmol, 36.03% yield) was obtained as a white solid.

LCMS: RT=2.893 min, MS cal.: 592.2, [M+H]⁺=593.2

HPLC: RT=11.180 min, purity: 97.2%

¹H NMR (400 MHz, MeOH-d4) δ=8.09 (d, J=1.2 Hz, 1H), 7.68 (s, 1H), 7.50 (t, J=8.4 Hz, 1H), 7.37 (dd, J=8.0, 10.4 Hz, 1H), 7.25 (t, J=74.4 Hz 1H), 7.23-7.17 (m, 2H), 6.81 (dd, J=2.8, 8.0 Hz, 1H), 5.50-5.45 (m, 2H), 4.02 (s, 3H), 3.97 (s, 2H), 3.09 (br d, J=11.2 Hz, 2H), 2.72-2.62 (m, 1H), 2.44-2.34 (m, 2H), 1.91-1.81 (m, 4H)

### Example A160—Preparation of Compound 243. 2-((4-(6-((4-Chloro-2-fluorobenzyl)oxy)-3-fluoropyridin-2-yl)piperidin-1-yl)methyl)-4-(difluoromethoxy)-1-methyl-1H-benzo[d]imidazole-6-carboxylic acid (Compound 243) (6)

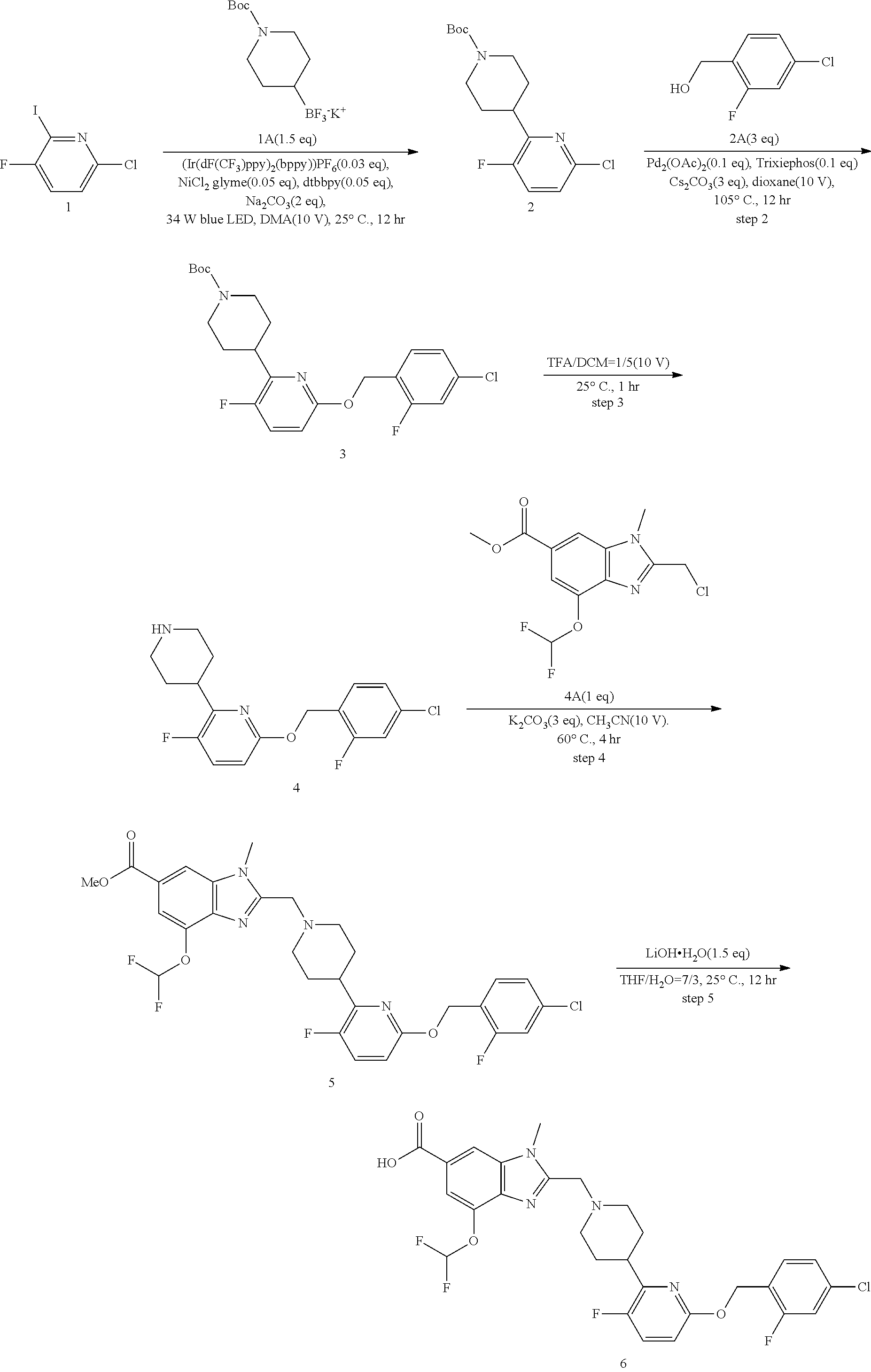

### General Procedure for Preparation of Intermediate 2:

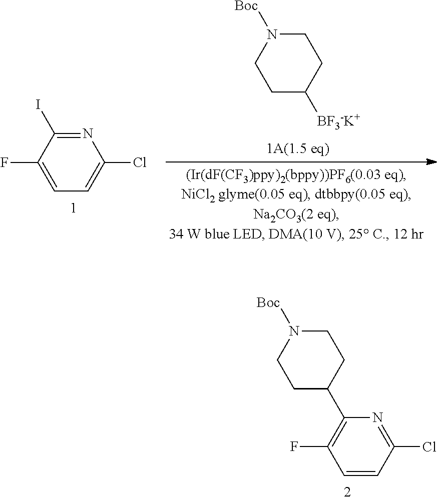

To a solution of 1A (848.26 mg, 2.91 mmol, 1.5 eq, K+) in DMA (3 mL) was added 1 (500 mg, 1.94 mmol, 1 eq), NiCl₂glyme (21.34 mg, 97.11 μmol, 0.05 eq), Na₂CO₃ (411.72 mg, 3.88 mmol, 2 eq), dtbbpy (26.07 mg, 97.11 μmol, 0.05 eq) and (Ir(dF(CF₃)ppy)₂(bpy))PF₆ (58.83 mg, 58.27 μmol, 0.03 eq). The mixture was stirred at 25° C. for 12 hr under 34W blue LED. TLC (Petroleum ether/Ethyl acetate=10/1) indicated 1 was consumed and one new spot formed. The reaction was clean according to TLC. 2 reactions were combined for workup. The reaction mixture was partitioned between H₂O 10 mL and EtOAc 30 mL. The organic phase was separated, washed with brine 10 mL, dried over Na₂SO₄, filtered and concentrated under reduced pressure to give a residue. The residue was purified by column chromatography (SiO₂, Petroleum ether/Ethyl acetate=1/0 to 10/1). 2 (480 mg, 1.52 mmol, 39% yield) was obtained as a colorless oil.

LCMS: RT=2.060 min, MS cal.: 314.1, 316.1, [M−55]⁺=259.1, 261.1

¹H NMR (400 MHz, CHCl3-d) δ=7.32 (t, J=8.8 Hz, 1H), 7.16 (dd, J=3.2, 8.4 Hz, 1H), 4.34-4.19 (m, 2H), 3.14 (t, J=11.2 Hz, 1H), 2.84 (t, J=12.4 Hz, 2H), 1.95-1.81 (m, 2H), 1.80-1.73 (m, 2H), 1.48 (s, 9H)

### General Procedure for Preparation of Intermediate 3:

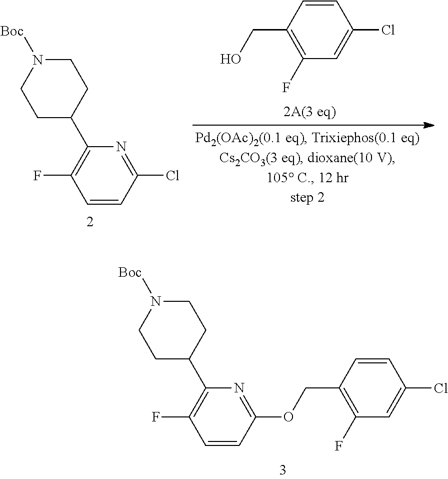

A mixture of 2 (240 mg, 762.43 μmol, 1 eq), 2A, Pd(OAc)₂ (17.12 mg, 76.24 μmol, 0.1 eq), Trixiephos (30.38 mg, 76.24 μmol, 0.1 eq) and Cs₂CO₃ (745.25 mg, 2.29 mmol, 3 eq) in dioxane (2.4 mL) was degassed and purged with N₂ for 3 times, and then the mixture was stirred at 105° C. for 12 hr under N₂ atmosphere. TLC (Petroleum ether/Ethyl acetate=10/1, product R_{f}=0.51) indicated 2 was consumed completely and one new spot formed. The reaction was clean according to TLC. The reaction mixture was filtered and diluted with H₂O 10 mL and extracted with EtOAc 15 mL (5 mL*3). The combined organic layers were washed with brine 5 mL, dried over Na₂SO₄, filtered and concentrated under reduced pressure to give a residue. The residue was purified by column chromatography (SiO₂, Petroleum ether/Ethyl acetate=1/0 to 15/1). 3 (240 mg, 366.38 μmol, 48% yield, 67% purity) was obtained as brown oil.

¹H NMR (400 MHz, CHCl3-d) δ=7.42 (t, J=8.0 Hz, 1H), 7.30 (s, 1H), 7.28 (s, 1H), 7.26 (s, 1H), 7.16-7.10 (m, 2H), 6.59 (dd, J=2.8, 8.8 Hz, 1H), 5.36 (s, 2H), 4.23 (d, J=12.4 Hz, 2H), 3.16-3.05 (m, 1H), 2.86 (t, J=11.6 Hz, 2H), 1.83 (dq, J=4.4, 12.4 Hz, 2H), 1.76-1.70 (m, 2H), 1.51 (s, 9H)

### General Procedure for Preparation of Compound 4:

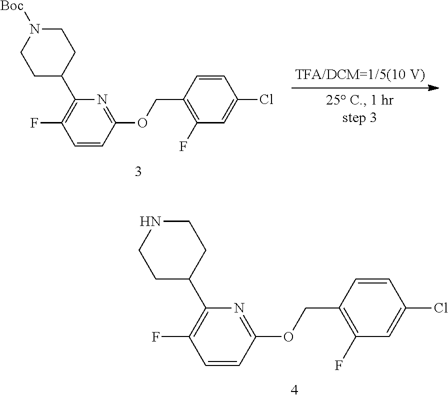 [SMILES: CC(C)(C)OC(=O)N1CCC(c2nc(OCc3ccc(Cl)cc3F)ccc2F)CC1 ; Fc1cc(Cl)ccc1COc1ccc(F)c(C2CCNCC2)n1]

To a solution of Compound 3 (100 mg, 152.66 μmol, 1 eq) in DCM (1 mL) was added TFA (307.00 mg, 2.69 mmol, 0.2 mL, 17.64 eq). The mixture was stirred at 25° C. for 1 hr. LC-MS (RT=1.394) showed Compound 3 was consumed completely and desired mass was detected. The reaction mixture was concentrated under reduced pressure to remove solvent. Without purification. Compound 4 (69 mg, TFA, crude) was obtained as a yellow oil.

LCMS: RT=1.394 min, MS cal.: 338.1, 340.1, [M+H]⁺=339.1, 341.1

### General Procedure for Preparation of Compound 5:

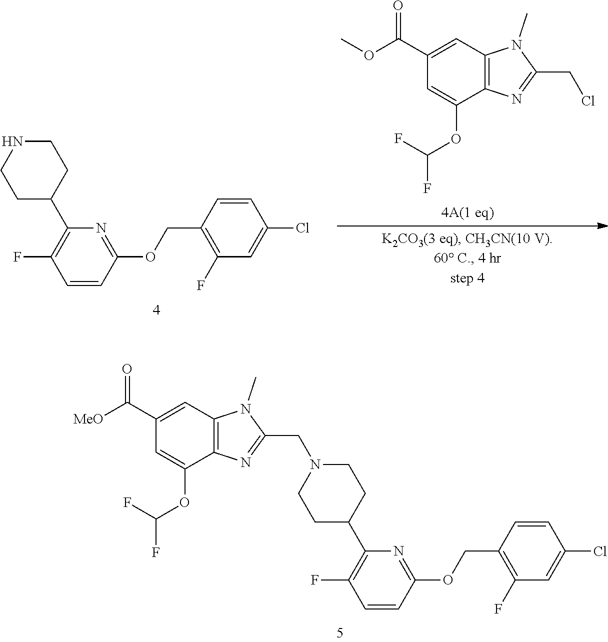 [SMILES: Fc1cc(Cl)ccc1COc1ccc(F)c(C2CCNCC2)n1 ; COC(=O)c1cc(OC(F)F)c2nc(CCl)n(C)c2c1 ; COC(=O)c1cc(OC(F)F)c2nc(CN3CCC(c4nc(OCc5ccc(Cl)cc5F)ccc4F)CC3)n(C)c2c1]

To a solution of Compound 4 (69 mg, TFA, crude) in ACN (0.5 mL) was added Compound 4A (46.43 mg, 152.38 μmol, 1 eq) and K₂CO₃ (21.06 mg, 152.38 μmol, 1 eq). The mixture was stirred at 60° C. for 2 hr. LC-MS (RT=1.726) showed Compound 4 was consumed almost and desired mass was detected. The reaction mixture was diluted with H₂O 10 mL and extracted with EtOAc 30 mL (10 mL*3). The combined organic layers were washed with brine 10 mL, dried over Na₂SO₄, filtered, and concentrated under reduced pressure to give a residue. The residue was purified by column chromatography (SiO₂, Petroleum ether/Ethyl acetate=20/1 to 3/1). Compound 5 (85 mg, 110.63 μmol, 72.60% yield, 79% purity) was obtained as a white solid.

LCMS: RT=1.727 min, MS cal.: 606.2, 608.2, [M+H]⁺=607.0, 609.0

### General Procedure for Preparation of Compound 243 (6):

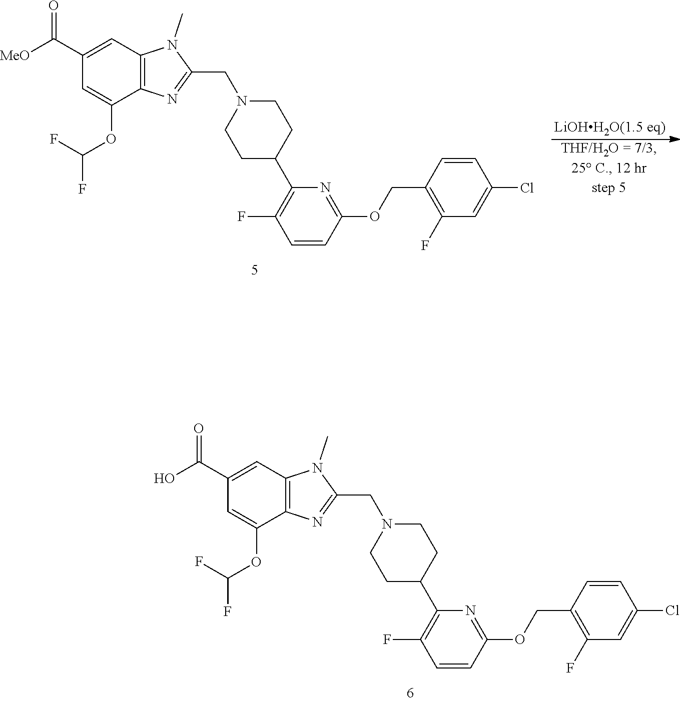

To a solution of 5 (75 mg, 97.61 μmol, 1 eq) in THF (0.7 mL) was added LiOH H₂O (6.14 mg, 146.42 μmol, 1.5 eq) in H₂O (0.3 mL). The mixture was stirred at 25° C. for 12 hr. LC-MS (RT=1.181) showed 5 was consumed almost and desired mass was detected. The mixture was purified by prep-HPLC (neutral condition, column: Waters Xbridge Prep OBD C18 150*40 mm*10 um; mobile phase: [H₂O (10 mM NH₄HCO₃)-ACN]; gradient: 10%-40% B over 8.0 min). 2-((4-(6-((4-Chloro-2-fluorobenzyl)oxy)-3-fluoropyridin-2-yl)piperidin-1-yl)methyl)-4-(difluoromethoxy)-1-methyl-1H-benzo[d]imidazole-6-carboxylic acid (28.58 mg, 47.98 μmol, 49.15% yield, 99.54% purity) was obtained as a white solid.

LCMS: RT=2.369 min, MS cal.: 592.2, 594.2, [M+H]⁺=593.2, 595.2

HPLC: RT=12.539 min, purity: 99.54%

¹H NMR (400 MHz, DMSO-d6) δ=8.07 (s, 1H), 7.63 (t, J=74.4 Hz, 1H), 7.60 (t, J=9.2 Hz, 1H), 7.57-7.51 (m, 2H), 7.47-7.42 (m, 1H), 7.27 (dd, J=1.6, 8.4 Hz, 1H), 6.73 (dd, J=2.8, 8.8 Hz, 1H), 5.33 (s, 2H), 3.97 (s, 3H), 3.86 (s, 2H), 2.97-2.86 (m, 3H), 2.23 (t, J=11.2 Hz, 2H), 1.87-1.73 (m, 2H), 1.71-1.62 (m, 2H):

### Example A161—Preparation of Compound 245. (S)-2-((4-(6-((4-Chloro-2-fluorobenzyl)oxy)pyridin-2-yl)piperidin-1-yl)methyl)-4-hydroxy-1-(oxetan-2-ylmethyl)-1H-benzo[d]imidazole-5-carboxylic acid (Compound 245) (5)

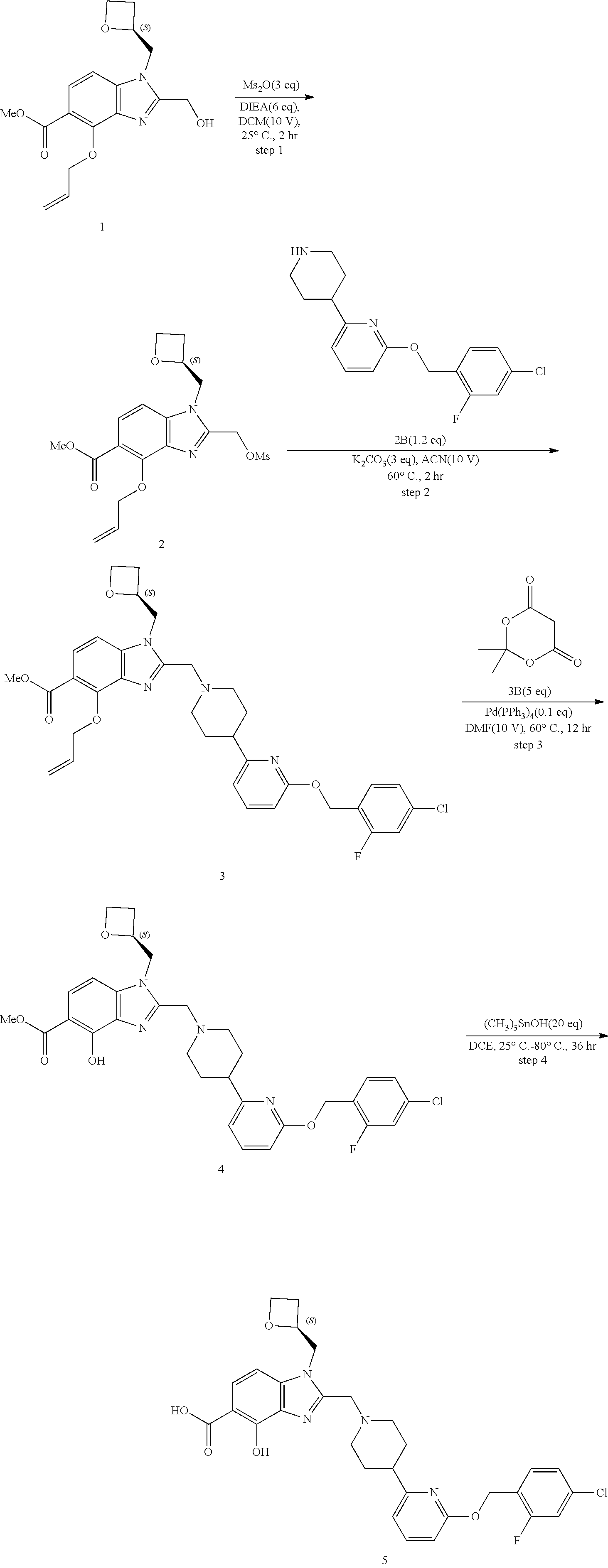

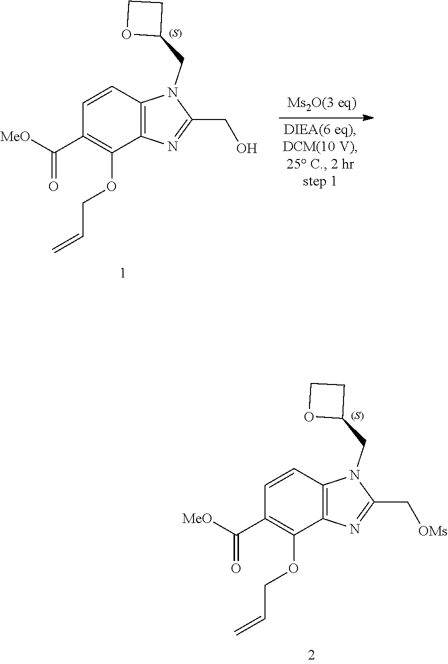

A mixture of 1 (300 mg, 902.66 μmol, 1 eq) (synthesized from Int 11), Ms₂O (471.72 mg, 2.71 mmol, 3 eq), DIEA (699.98 mg, 5.42 mmol, 943.36 μL, 6 eq) in DCM (3 mL) was degassed and purged with N₂ for 3 times, and then the mixture was stirred at 25° C. for 2 hr under N₂ atmosphere. LCMS (product RT=1.040 min) showed 1 was consumed completely and one main peak with desired mass was detected. The reaction mixture was added HCOOH at 25° C. until pH=7-8, and then diluted with H₂O 10 mL and extracted with DCM (10 mL*4). The combined organic layers were dried over Na₂SO₄, filtered and concentrated under reduced pressure to give a residue. 2 (370 mg, crude) was obtained as a yellow oil.

### General Procedure for Preparation of Intermediate 3:

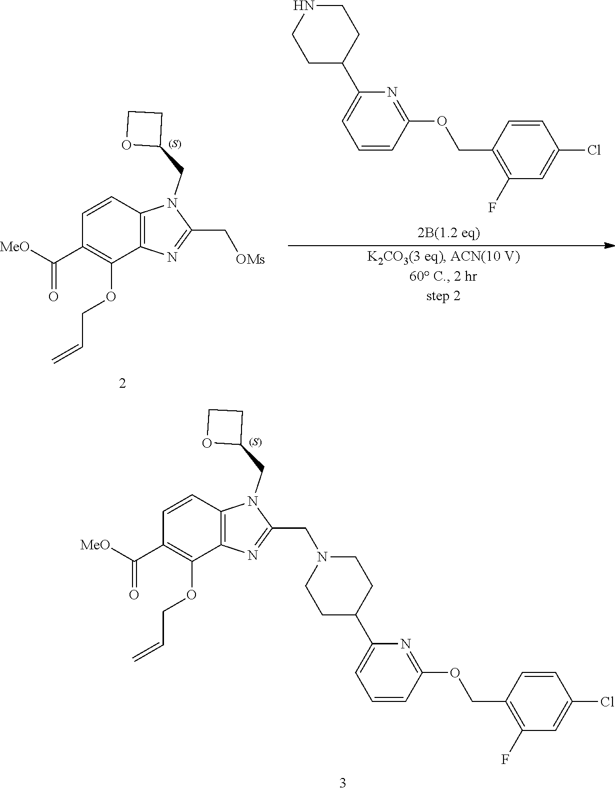

A mixture of 2 (370 mg, 901.47 μmol, 1 eq), 2B (347.02 mg, 1.08 mmol, 1.2 eq), K₂CO₃ (373.76 mg, 2.70 mmol, 3 eq) in ACN (3.7 mL) was degassed and purged with N₂ for 3 times, and then the mixture was stirred at 60° C. for 2 hr under N₂ atmosphere. TLC (Petroleum ether/Ethyl acetate=0/1, R_{f}=0.23) indicated 2 was consumed completely and one new spot formed. The reaction mixture was diluted with H₂O 10 mL and extracted with DCM (10 mL*4). The combined organic layers were dried over Na₂SO₄, filtered, and concentrated under reduced pressure to give a residue. The residue was purified by column chromatography (SiO₂, Petroleum ether/Ethyl acetate=100/1 to 0/1). 3 (350 mg, 495.97 μmol, 55% yield, 90% purity) was obtained as a yellow oil.

LCMS: RT=1.723 min, MS cal.: 635.1, [M+H]⁺=635.0

¹H NMR (400 MHz, DMSO-d₆) δ=7.63 (t, J=7.6 Hz, 1H), 7.59-7.53 (m, 2H), 7.46 (dd, J=2.0, 10.0 Hz, 1H), 7.35 (d, J=8.8 Hz, 1H), 7.29 (dd, J=2.0, 8.0 Hz, 1H), 6.87 (d, J=7.2 Hz, 1H), 6.67 (d, J=8.0 Hz, 1H), 6.17-6.00 (m, 1H), 5.45-5.39 (m, 1H), 5.39-5.35 (m, 2H), 5.23 (d, J=5.2 Hz, 2H), 5.21-5.15 (m, 1H), 5.10 (dd, J=2.8, 7.2 Hz, 1H), 4.72 (d, J=7.2 Hz, 1H), 4.63-4.56 (m, 1H), 4.50-4.44 (m, 1H), 4.40 (td, J=6.0, 9.2 Hz, 1H), 3.93 (d, J=13.6 Hz, 1H), 3.82-3.73 (m, 4H), 2.99 (d, J=11.2 Hz, 1H), 2.89-2.82 (m, 1H), 2.74-2.66 (m, 1H), 2.65-2.56 (m, 1H), 2.46-2.39 (m, 1H), 2.29-2.14 (m, 2H), 1.86-1.65 (m, 4H)

### General Procedure for Preparation of Intermediate 4:

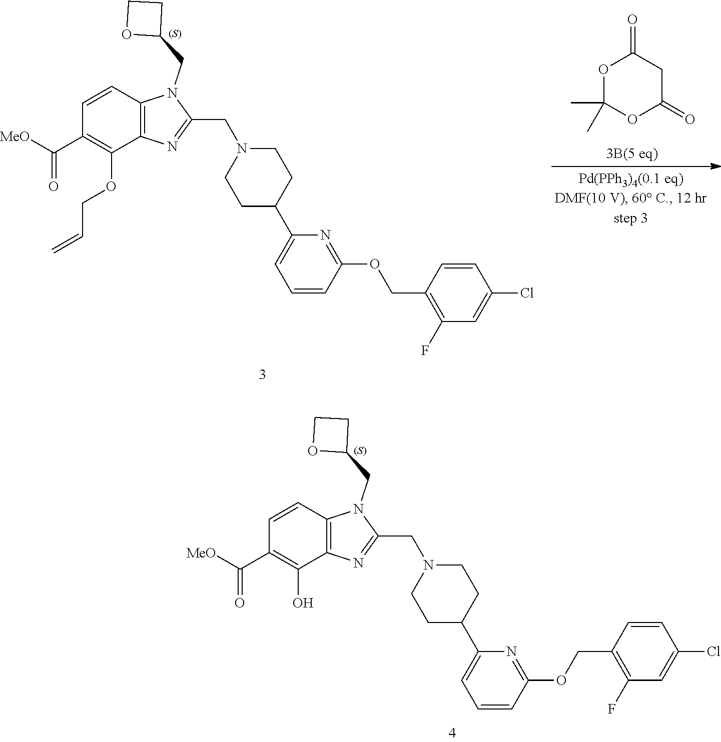

A mixture of 3 (220 mg, 346.39 μmol, 1 eq), 3B (249.62 mg, 1.73 mmol, 5 eq), Pd(PPh₃)₄ (40.03 mg, 34.64 μmol, 0.1 eq) in DMF (2.2 mL) was degassed and purged with N₂ for 3 times, and then the mixture was stirred at 50° C. for 12 hr under N₂ atmosphere. LCMS (product RT=1.626 min) showed 3 was consumed completely and one main peak with desired mass was detected. The reaction mixture was filtered. The filtrate was purified by prep-HPLC (column: Waters Xbridge Prep OBD C₁₈ 150*40 mm*10 um; mobile phase: [H₂O (10 mM NH₄HCO₃)-ACN]; gradient: 30%-85% B over 8.0 min). 4 (60 mg, 98.81 μmol, 20% yield, 98% purity) was obtained as a pink solid.

LCMS: RT=1.641 min, MS cal.: 595.1, [M+H]⁺=595.0

¹H NMR (400 MHz, DMSO-d₆) δ=11.28 (br s, 1H), 7.67-7.60 (m, 2H), 7.56 (t, J=8.0 Hz, 1H), 7.45 (dd, J=2.0, 10.0 Hz, 1H), 7.29 (dd, J=20, 8.4 Hz, 1H), 7.20 (d, J=8.8 Hz, 1H), 6.87 (d, J=7.2 Hz, 1H), 6.67 (d, J=8.0 Hz, 1H), 5.37 (s, 2H), 5.10 (dq, J=3.2, 7.2 Hz, 1H), 4.76-4.68 (m, 1H), 4.63-4.56 (m, 1H), 4.50-4.42 (m, 1H), 4.37 (td, J=6.0, 9.2 Hz, 1H), 3.95-3.86 (m, 4H), 3.76 (d, J=13.6 Hz, 1H), 2.98 (d, J=11.2 Hz, 1H), 2.85 (d, J=11.2 Hz, 1H), 2.73-2.65 (m, 1H), 2.64-2.58 (m, 1H), 2.47-2.37 (m, 1H), 2.27-2.12 (m, 2H), 1.84-1.65 (m, 4H)

### General Procedure for Preparation of Compound 245 (5):

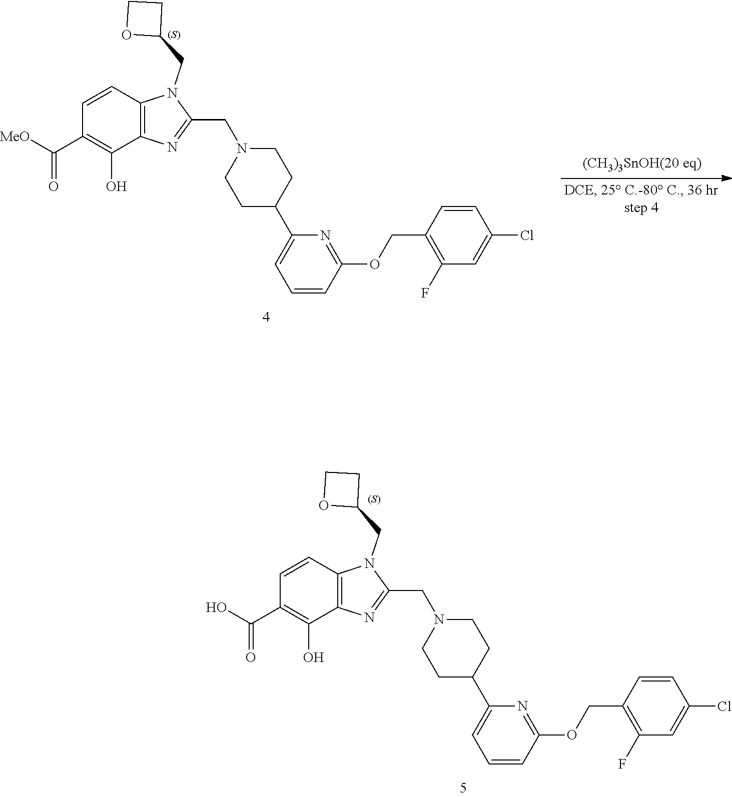

DCE (0.5 mL) was charged to the three-necked round bottom flask, then 4 (50 mg, 84.03 μmol, 1 eq) was added to the mixture at 25° C. At 25° C. (inner temperature), (CH₃)₃SnOH (303.87 mg, 1.68 mmol, 20 eq) was added to the reaction mixture at 25° C. After the addition, the mixture was stirred at 80° C. for 12 hr. The mixture was stirred at 80° C. for 24 hr. LCMS (product RT=1.169 min) showed 4 was consumed completely and one main peak with desired mass was detected. The reaction mixture was added saturated KF solution (3 ml) at 25° C., the mixture was diluted with H₂O 5 mL and extracted with DCM (10 mL*4). The combined organic layers were dried over Na₂SO₄, filtered, and concentrated under reduced pressure to give a residue. The residue was purified by prep-HPLC (column: Phenomenex Luna C₁₈ 100*30 mm*3 um; mobile phase: [H₂O (0.2% FA)-ACN]; gradient: 20%-60% B over 8.0 min). (S)-2-((4-(6-((4-Chloro-2-fluorobenzyl)oxy)pyridin-2-yl)piperidin-1-yl)methyl)-4-hydroxy-1-(oxetan-2-ylmethyl)-1H-benzo[d]imidazole-5-carboxylic acid (28.13 mg, 48.41 μmol, 57% yield) was obtained as a white solid.

LCMS: RT=2.309 min, MS cal.: 581.0, [M+H]⁺=581.2

HPLC: RT=11.575 min, purity: 97.79%

¹H NMR (400 MHz, MeOH-d4) δ=7.83 (d, J=8.8 Hz, 1H), 7.67-7.58 (m, 1H), 7.49 (t, J=8.0 Hz, 1H), 7.27-7.15 (m, 2H), 6.97 (d, J=8.8 Hz, 1H), 6.88 (d, J=7.2 Hz, 1H), 6.68 (d, J=8.0 Hz, 1H), 5.42 (s, 2H), 5.28-5.18 (m, 1H), 4.74-4.62 (m, 2H), 4.62-4.54 (m, 1H), 4.46 (s, 2H), 4.41 (td, J=5.6, 9.2 Hz, 1H), 3.62-3.47 (m, 2H), 3.07-2.94 (m, 2H), 2.94-2.85 (m, 1H), 2.82-2.70 (m, 1H), 2.56-2.43 (m, 1H), 2.15-2.01 (m, 4H)

### Example A162—Preparation of Compound 246. 2-((4-(6-((4-Chloro-2-(difluoromethoxy)benzyl)oxy)pyridin-2-yl)piperidin-1-yl)methyl)-4-(difluoromethoxy)-1-methyl-1H-benzo[d]imidazole-6-carboxylic acid (Compound 246) (5)

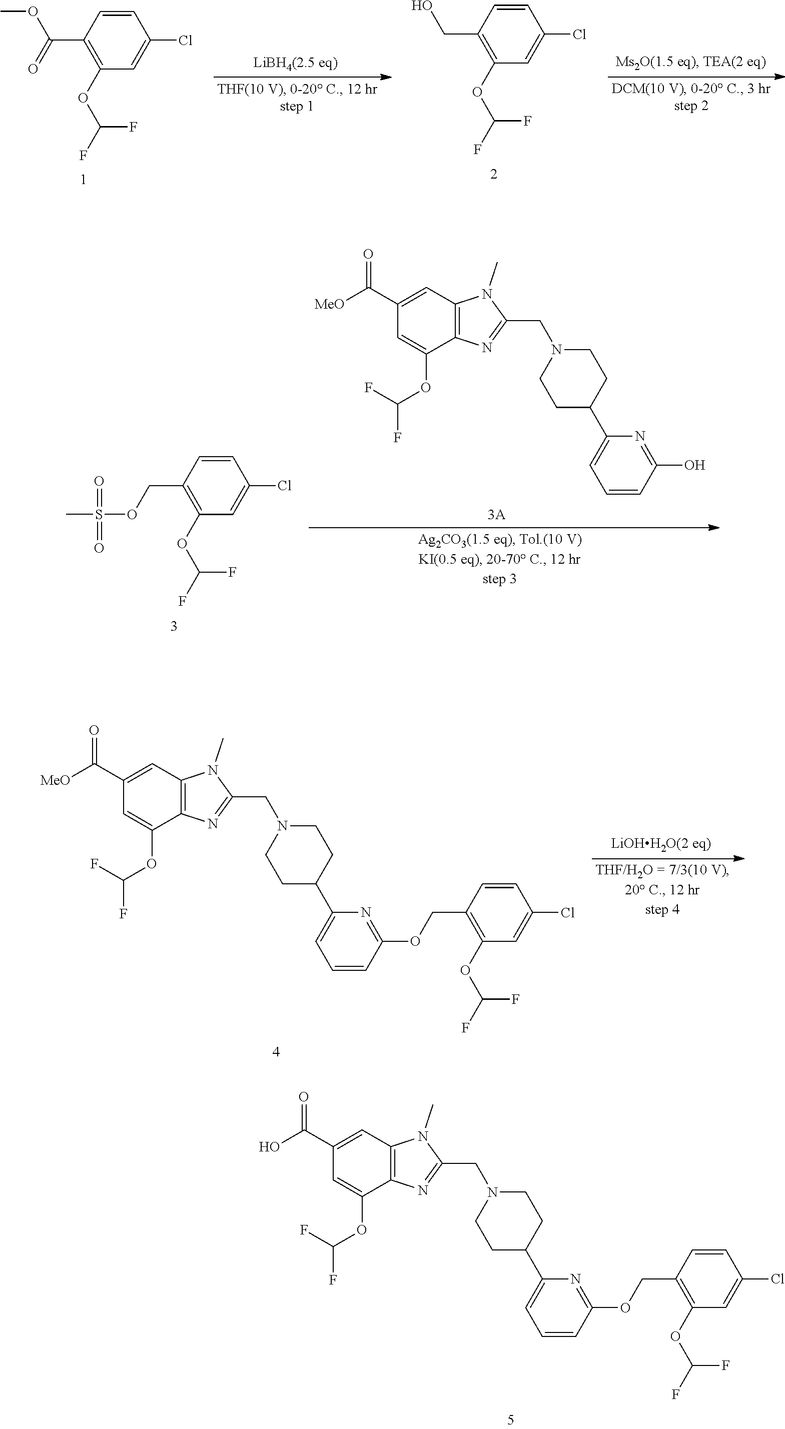 [SMILES: COC(=O)c1ccc(Cl)cc1OC(F)F ; OCc1ccc(Cl)cc1OC(F)F ; CS(=O)(=O)OCc1ccc(Cl)cc1OC(F)F ; COC(=O)c1cc(OC(F)F)c2nc(CN3CCC(c4cccc(O)n4)CC3)n(C)c2c1 ; COC(=O)c1cc(OC(F)F)c2nc(CN3CCC(c4cccc(OCc5ccc(Cl)cc5OC(F)F)n4)CC3)n(C)c2c1 ; Cn1c(CN2CCC(c3cccc(OCc4ccc(Cl)cc4OC(F)F)n3)CC2)nc2c(OC(F)F)cc(C(=O)O)cc21]

### General Procedure for Preparation of Intermediate 2:

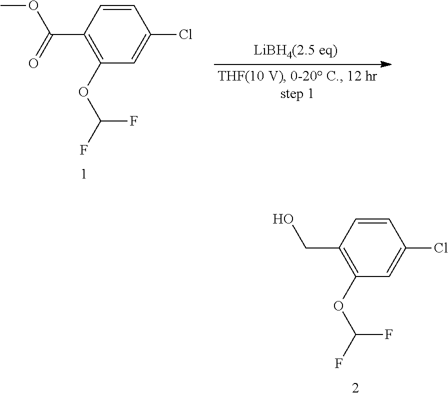

Equip a 50 mL round bottom flask. THF (9 mL) was charged to the round bottom flask, then 1 (900 mg, 3.80 mmol, 1 eq) was added to the mixture at 20° C. At 0° C. (inner temperature) LiBH₄ (2 M, 4.75 mL, 2.5 eq) was added to the reaction mixture at 0° C. under N₂ atmosphere. After the addition, the suspension was degassed and purged with N₂ for 3 times, and then the mixture was stirred under N₂ atmosphere at 20° C. for 12 hr TLC (Petroleum ether:Ethyl acetate=3:1, product R_{f}=0.42) indicated 1 was consumed completely and one new spot formed. The reaction was clean according to TLC. The reaction mixture was quenched by addition a.q. NaHCO₃15 mL at 20° C., and then diluted with H₂O 15 mL and extracted with DCM 15 mL. The combined organic layers were washed with brine 5 mL, dried over Na₂SO₄, filtered, and concentrated under reduced pressure to give a residue. 2 (720 mg, 3.45 mmol, 91% yield) was obtained as a colorless oil.

¹H NMR (400 MHz, CHCl3-d) δ=7.54 (d, J=8.0 Hz, 1H), 7.40-7.31 (m, 1H), 6.85-6.49 (t, J=72.0 Hz, 1H), 4.83 (s, 2H)

### General Procedure for Preparation of Intermediate 3:

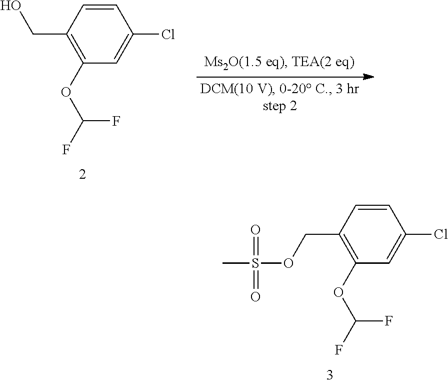 [SMILES: OCc1ccc(Cl)cc1OC(F)F ; CS(=O)(=O)OCc1ccc(Cl)cc1OC(F)F]

Equip a 25 mL three-neck bottle. DCM (2 mL) was charged to the three-neck bottle, then 2 (200 mg, 958.82 μmol, 1 eq) methylsulfonyl methanesulfonate (250.53 mg, 1.44 mmol, 1.5 eq) was added to the mixture at 20° C. At 0° C. (inner temperature) TEA (194.04 mg, 1.92 mmol, 266.91 μL, 2 eq) was added to the reaction mixture at 0° C. under N₂ atmosphere. After the addition, the suspension was degassed and purged with N₂ for 3 times, and then the mixture was stirred under N₂ atmosphere at 20° C. for 3 hr. LC-MS showed 2 was consumed completely and one main peak with desired mass was detected. The reaction mixture was poured into ice water 10 mL slowly at 0° C., and then extracted with DCM 15 mL. The combined organic layers were washed with brine 15 mL, dried over Na₂SO₄, filtered, and concentrated under reduced pressure to give a residue. 3 (200 mg, 697.64 μmol, 73% yield) was obtained as a colorless oil.

LCMS: RT=0.484 min, MS cal.: 286.0, 280.0

¹H NMR (400 MHz, CHCl3-d) δ=7.45 (d, J=8.0 Hz, 1H), 7.28 (d, J=2.0 Hz, 1H), 7.26 (d, J=2.0 Hz, 1H), 7.23 (s, 1H), 6.59 (t, J=72.0 Hz, 1H), 5.26 (s, 2H), 3.03 (s, 3H)

### General Procedure for Preparation of Intermediate 4:

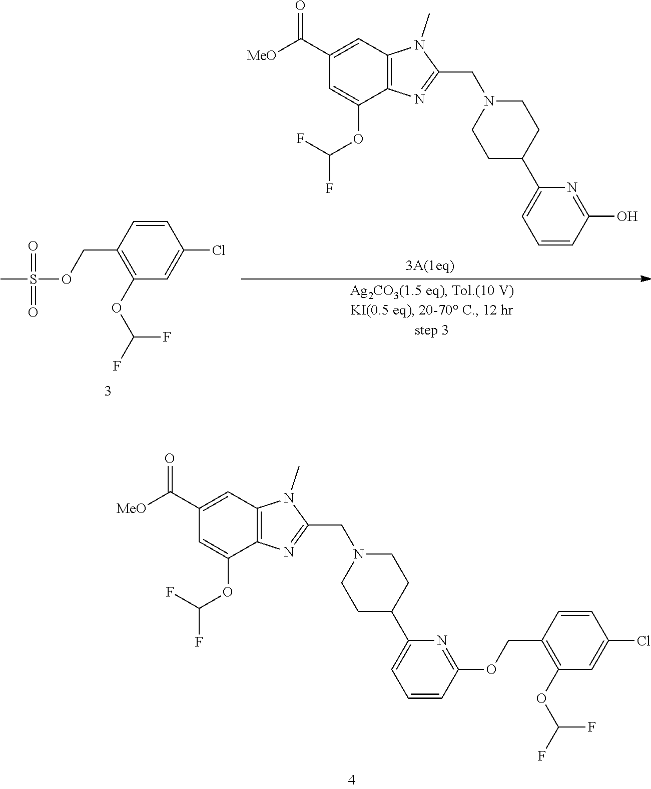 [SMILES: CS(=O)(=O)OCc1ccc(Cl)cc1OC(F)F ; COC(=O)c1cc(OC(F)F)c2nc(CN3CCC(c4cccc(O)n4)CC3)n(C)c2c1 ; COC(=O)c1cc(OC(F)F)c2nc(CN3CCC(c4cccc(OCc5ccc(Cl)cc5OC(F)F)n4)CC3)n(C)c2c1]

To a solution of 3 (96.32 mg, 335.99 μmol, 1.5 eq) 3A (100 mg, 223.99 μmol, 1 eq) in toluene (1 mL) was added Ag₂CO₃ (123.53 mg, 447.98 μmol, 20.33 μL, 2 eq) and KI (18.59 mg, 112.00 μmol, 0.5 eq). The mixture was stirred at 100° C. for 12 hr. LC-MS showed 3 was consumed completely and the desired mass was detected as a main peak. The suspension was filtered with celite, and the filter cake was dissolved in EtOAc (15 mL), washed with brine 10 mL, dried over Na₂SO₄, filtered and the filtrate was concentrated to give a crude product. The filtrate was extracted with EtOAc 15 mL, washed with water 15 mL, brine 15 mL, dried over Na₂SO₄, filtered and the filtrate was concentrated to give a crude product. The crude products were combined. The residue was purified by column chromatography (SiO₂, Petroleum ether/Ethyl acetate=1/1). 4 (80 mg, 125.58 μmol, 56% yield) was obtained as a brown oil.

LCMS: RT=0.770 min, MS cal.: 636.2, 637.2, [M+H]⁺=637.2, 638.2

¹H NMR (400 MHz, DMSO-d6) δ=8.11 (d, J=1.2 Hz, 1H), 7.66-7.60 (m, 2H), 7.65 (t, J=72.8 Hz, 1H), 7.51-7.57 (m, 3H), 7.34 (t, J=72.8 Hz, 1H), 7.37-7.30 (m, 3H), 6.86 (d, J=7.2 Hz, 1H), 6.67 (d, J=8.0 Hz, 1H), 5.34 (s, 2H), 3.97 (s, 3H), 3.94 (s, 1H), 3.90 (s, 3H), 3.85 (s, 2H), 2.93 (d, J=10.0 Hz, 2H), 2.54-2.62 (m, 1H), 2.21 (t, J=10.0 Hz, 2H), 1.62-1.82 (m, 4H)

### General Procedure for Preparation of Compound 246 (5):

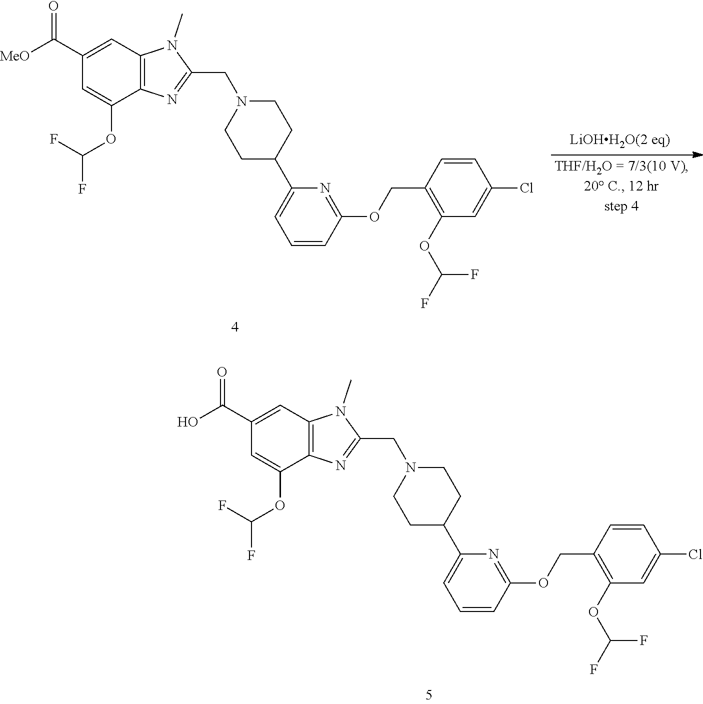 [SMILES: COC(=O)c1cc(OC(F)F)c2nc(CN3CCC(c4cccc(OCc5ccc(Cl)cc5OC(F)F)n4)CC3)n(C)c2c1 ; Cn1c(CN2CCC(c3cccc(OCc4ccc(Cl)cc4OC(F)F)n3)CC2)nc2c(OC(F)F)cc(C(=O)O)cc21]

To a solution of 4 (70 mg, 109.89 μmol, 1 eq) in THF (0.7 mL) was added LiOH·H₂O (9.22 mg, 219.77 μmol, 2 eq) in H₂O (0.3 mL). The mixture was stirred at 20° C. for 12 hr. LC-MS showed 4 was consumed completely and one main peak with desired mass was detected. The mixture was purified directly. The residue was purified by prep-HPLC (column: Waters Xbridge Prep OBD C18 150*40 mm*10 um; mobile phase: [H₂O (10 mM NH₄HCO₃)-ACN]; gradient: 25%-65% B over 8.0 min). 2-((4-(6-((4-Chloro-2-(difluoromethoxy)benzyl)oxy)pyridin-2-yl)piperidin-1-yl)methyl)-4-(difluoromethoxy)-1-methyl-1H-benzo[d]imidazole-6-carboxylic acid (21.44 mg, 34.41 μmol, 31% yield) was obtained as a white solid.

LCMS: RT=2.847 min, MS cal.: 622.2, 624.2, [M+H]⁺=623.2, 625.2

¹H NMR (400 MHz, DMSO-d6) δ=8.05 (s, 1H), 7.66-7.59 (m, 1H), 7.62 (t, J=72.8 Hz, 1H), 7.55 (d, J=8.0 Hz, 1H), 7.51 (s, 1H), 7.36 (s, 1H), 7.33 (t, J=72.8 Hz, 1H), 7.31 (d, J=2.0 Hz, 1H), 6.86 (d, J=7.2 Hz, 1H), 6.67 (d, J=8.0 Hz, 1H), 5.34 (s, 2H), 3.95 (s, 3H), 3.84 (s, 2H), 2.93 (d, J=11.0 Hz, 2H), 2.61-2.56 (m, 1H), 2.21 (t, J=11.0 Hz, 2H), 1.82-1.74 (m, 2H), 1.74-1.63 (m, 2H)

### Example A163—Preparation of Compound 247. 2-((4-(6-((5-Chloropyridin-2-yl)methoxy)pyridin-2-yl)piperidin-1-yl)methyl)-4-(difluoromethoxy)-1-methyl-1H-benzo[d]imidazole-6-carboxylic acid (Compound 247) (3)

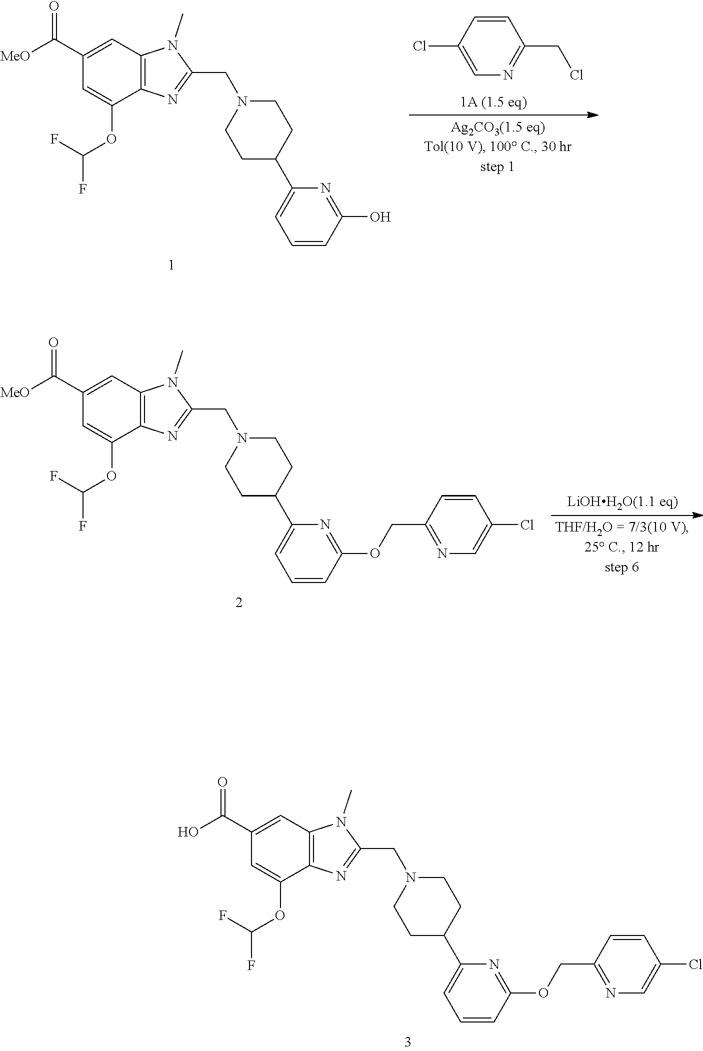 [SMILES: COC(=O)c1cc(OC(F)F)c2nc(CN3CCC(c4cccc(O)n4)CC3)n(C)c2c1 ; ClCc1ccc(Cl)cn1 ; COC(=O)c1cc(OC(F)F)c2nc(CN3CCC(c4cccc(OCc5ccc(Cl)cn5)n4)CC3)n(C)c2c1 ; Cn1c(CN2CCC(c3cccc(OCc4ccc(Cl)cn4)n3)CC2)nc2c(OC(F)F)cc(C(=O)O)cc21]

### General Procedure for Preparation of Intermediate 2:

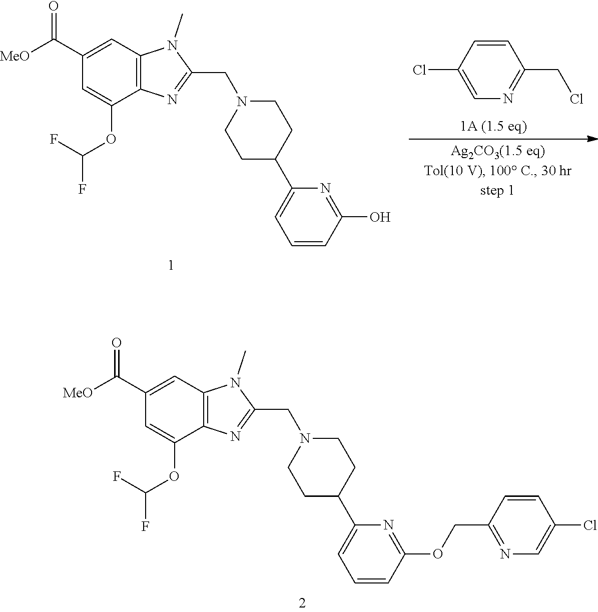 [SMILES: COC(=O)c1cc(OC(F)F)c2nc(CN3CCC(c4cccc(O)n4)CC3)n(C)c2c1 ; ClCc1ccc(Cl)cn1 ; COC(=O)c1cc(OC(F)F)c2nc(CN3CCC(c4cccc(OCc5ccc(Cl)cn5)n4)CC3)n(C)c2c1]

A mixture of 1 (100 mg, 223.99 μmol, 1 eq), 1A (54.44 mg, 335.99 μmol, 1.5 eq), Ag₂CO₃ (92.64 mg, 335.99 μmol, 15.25 μL, 1.5 eq), in Toluene (1 mL) was degassed and purged with N₂ for 3 times, and then the mixture was stirred at 100° C. for 48 hr under N₂ atmosphere. LC-MS showed 1 was consumed completely and one main peak with desired mass was detected. The residue was poured into water (30 mL). The aqueous phase was extracted with ethyl acetate (10 mL*3). The combined organic phase was washed with saturated brine (30 mL*1), dried with anhydrous Na₂SO₄, filtered and concentrated in vacuum. The residue was purified by prep-TLC (SiO₂, Petroleum ether:Ethyl acetate-3:1, Rf=0.50). 2 (45 mg, 63.61 μmol, 28.40% yield) was obtained as a yellow oil.

LCMS: RT=0.475 min, MS cal.: 571.18, [M+H]⁺=572.3

HPLC: product: RT=2.704 min, purity: 80.85%

### General Procedure for Preparation of Compound 247 (3):

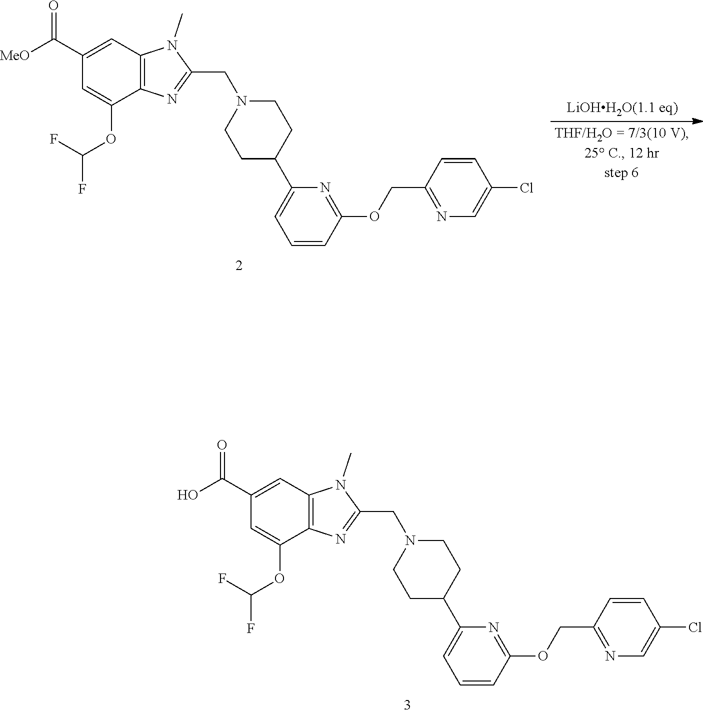 [SMILES: COC(=O)c1cc(OC(F)F)c2nc(CN3CCC(c4cccc(OCc5ccc(Cl)cn5)n4)CC3)n(C)c2c1 ; Cn1c(CN2CCC(c3cccc(OCc4ccc(Cl)cn4)n3)CC2)nc2c(OC(F)F)cc(C(=O)O)cc21]

To a solution of 2 (40.00 mg, 69.93 μmol, 1 eq) in THF (0.7 mL) and H₂O (0.3 mL) was added LiOH·H₂O (3.52 mg, 83.92 μmol, 1.2 eq). The mixture was stirred at 25° C. for 15 hr. LC-MS showed 2 was consumed completely and one main peak with desired mass was detected. The mixture was filtered and concentrated in vacuum. The residue was purified by prep-HPLC (column: Waters Xbridge Prep OBD C18 150*40 mm*10 um; mobile phase: [H₂O (10 mM NH₄HCO₃)-ACN]; gradient: 15%-65% B over 8.0 min). 2-((4-(6-((5-Chloropyridin-2-yl)methoxy)pyridin-2-yl)piperidin-1-yl)methyl)-4-(difluoromethoxy)-1-methyl-1H-benzo[d]imidazole-6-carboxylic acid (20.02 mg, 35.20 μmol, 50.34% yield) was obtained as a white solid.

LCMS: RT=2.540 min, MS cal.: 557.2, [M+H]⁺=558.2.

HPLC: RT=10.109 min, purity: 98.11%

1H NMR (400 MHz, MeOH-d4) δ=8.49 (d, J=2.4 Hz, 1H), 8.09 (s, 1H), 7.81 (dd, J=2.4, 8.5 Hz, 1H), 7.67 (s, 1H), 7.59 (t, J=7.7 Hz, 1H), 7.48 (d, J=8.3 Hz, 1H), 7.24 (t, J=74.4 Hz, 1H), 6.83 (d, J=7.3 Hz, 1H), 6.71 (d, J=8.1 Hz, 1H), 5.44 (s, 2H), 4.00 (s, 3H), 3.97 (s, 2H), 3.07 (d, J=11.4 Hz, 2H), 2.69-2.58 (m, 1H), 2.40 (dt, J=4.5, 10.8 Hz, 2H), 1.87-1.74 (m, 4H)

### Example A164—Preparation of Compound 248. 2-((4-(6-((2-Chlorobenzyl)oxy)pyridin-2-yl)piperidin-1-yl)methyl)-4-(difluoromethoxy)-1-methyl-1H-benzo[d]imidazole-6-carboxylic acid (Compound 248) (7)

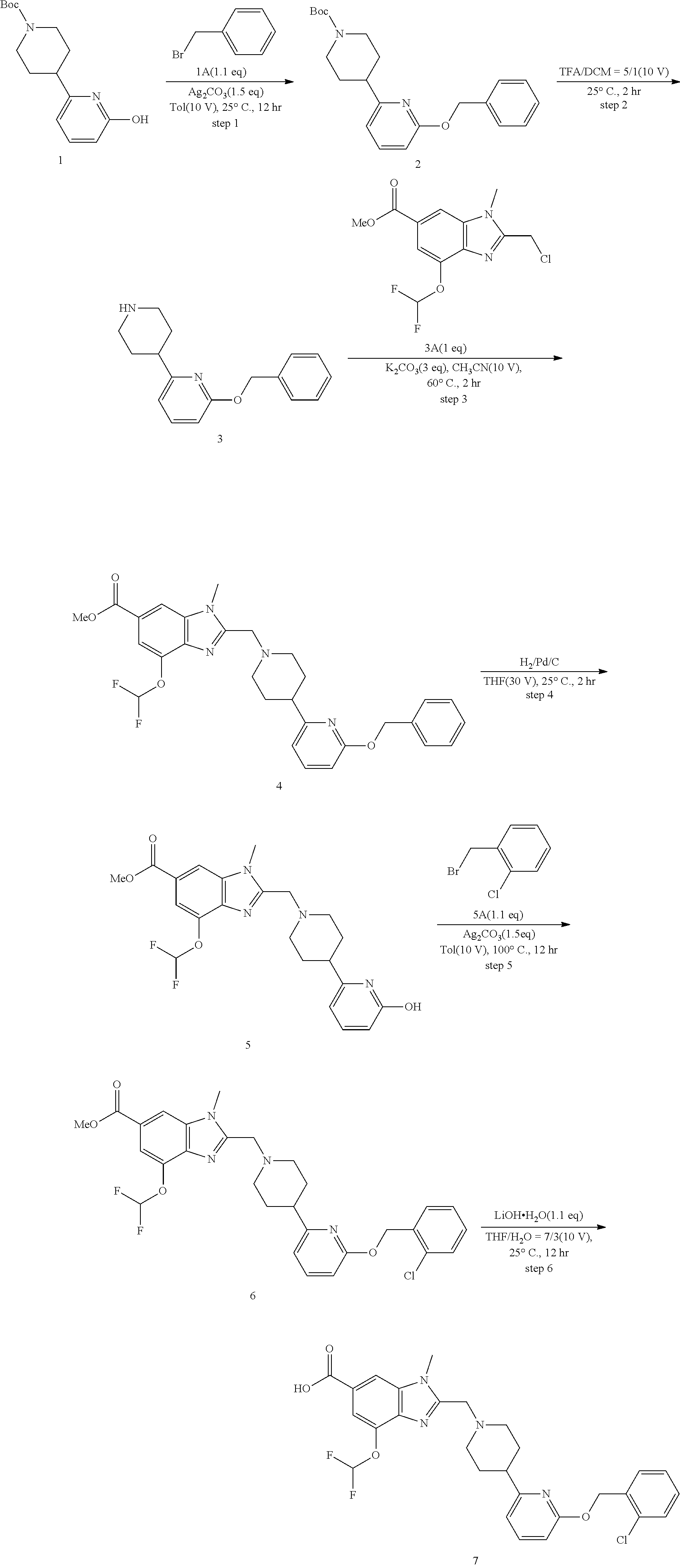

### General Procedure for Preparation of Intermediate 2:

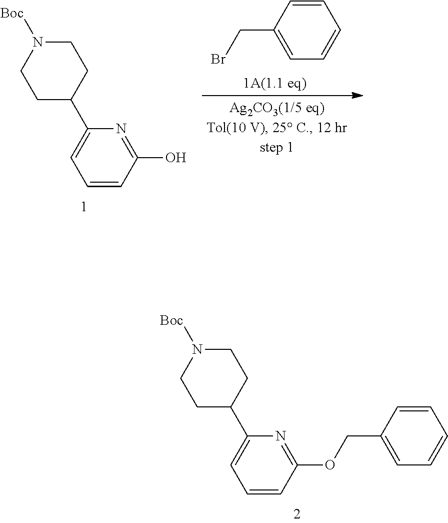 [SMILES: CC(C)(C)OC(=O)N1CCC(c2cccc(O)n2)CC1 ; BrCc1ccccc1 ; CC(C)(C)OC(=O)N1CCC(c2cccc(OCc3ccccc3)n2)CC1]

To a mixture of 1 (4.5 g, 16.17 mmol, 1 eq) in Toluene (45 mL) was added 1A (3.04 g, 17.78 mmol, 2.11 mL, 1.1 eq) and Ag₂CO₃ (6.69 g, 24.25 mmol, 1.10 mL, 1.5 eq). The mixture was stirred at 100° C. for 12 hr. LC-MS showed 1 was consumed completely and one main peak with desired mass was detected. The reaction mixture was quenched by H₂O 50 mL, then extracted with EA 60 mL (20 mL*3). The combined organic layers were washed with brine 30 mL, dried over Na₂SO₄, filtered, and concentrated under reduced pressure to give a residue. The residue was purified by column chromatography (SiO₂, Petroleum ether/Ethyl acetate=100/1 to 100/5). 2 (5.1 g, 13.84 mmol, 85.61% yield) was obtained as a white solid.

LCMS: RT=0.932 min, MS cal.: 368.2, [M+H]⁺=369.3

¹H NMR (400 MHz, CHCl3-d) δ=7.63 (t, J=7.6 Hz, 1H), 7.47-7.42 (m, 2H), 7.39-7.28 (m, 3H), 6.85 (d, J=7.2 Hz, 1H), 6.67 (d, J=8.0 Hz, 1H), 5.33 (s, 2H), 4.04 (br d, J=12.0 Hz, 2H), 2.91-2.71 (m, 3H), 1.79 (br d, J=11.2 Hz, 2H), 1.57 (dq, J=4.0, 12.4 Hz, 2H), 1.45-1.39 (m, 9H)

### General Procedure for Preparation of Intermediate 3:

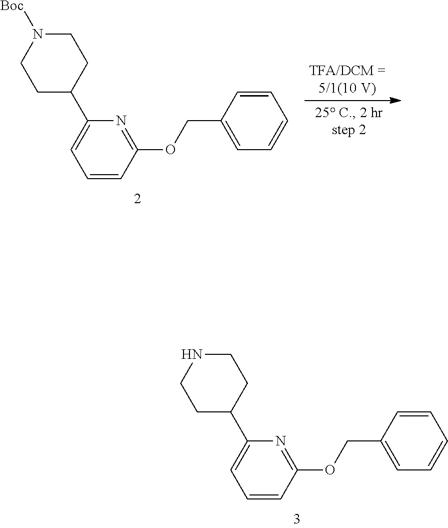 [SMILES: CC(C)(C)OC(=O)N1CCC(c2cccc(OCc3ccccc3)n2)CC1 ; c1ccc(COc2cccc(C3CCNCC3)n2)cc1]

To a mixture of 2 (5.1 g, 13.84 mmol, 1 eq) in TFA (8.5 mL) and DCM (42.5 mL). The mixture was stirred at 20° C. for 1 hr. LC-MS showed 2 was consumed completely and one main peak with desired mass was detected. The mixture was concentrated under reduced pressure to afford a yellow liquid. Then the liquid was basified to pH=8-9 with saturated aqueous NaHCO₃. Then the mixture was extracted with DCM (20 mL*3). The combined organic phases were washed with brine (50 mL), dried with anhydrous Na₂SO₄, filtered and the filtrate was concentrated in vacuum to afford a yellow residue. The residue was used in the next step without further purification. 3 (4.2 g, crude) was obtained as a white solid.

LCMS: RT=0.588 min, MS cal.: 268.2, [M+H]⁺=269.3

¹H NMR (400 MHz, CHCl3-d) δ=7.63 (dd, J=7.6, 8.4 Hz, 1H), 7.44-7.40 (m, 2H), 7.37-7.32 (m, 2H), 7.31-7.26 (m, 1H), 6.86 (d, J=7.2 Hz, 1H), 6.71 (d, J=8.4 Hz, 1H), 5.39 (s, 2H), 3.48 (td, J=3.2, 12.8 Hz, 2H), 3.13 (dt, J=4.0, 12.4 Hz, 2H), 3.03-2.94 (m, 1H), 2.15-1.99 (m, 4H)

### General Procedure for Preparation of Intermediate 4:

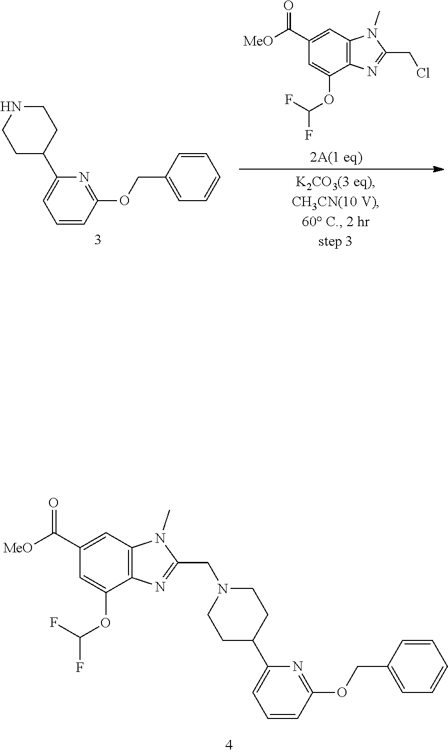 [SMILES: c1ccc(COc2cccc(C3CCNCC3)n2)cc1 ; COC(=O)c1cc(OC(F)F)c2nc(CCl)n(C)c2c1 ; COC(=O)c1cc(OC(F)F)c2nc(CN3CCC(c4cccc(OCc5ccccc5)n4)CC3)n(C)c2c1]

To a mixture of 3 (2.47 g, 9.19 mmol, 1.4 eq) and 2A (2 g, 6.56 mmol, 1 eq) in ACN (23 mL) was added K₂CO₃ (2.72 g, 19.69 mmol, 3 eq) under N₂. The mixture was stirred at 60° C. for 4 hr. LC-MS showed 3 was consumed completely and one main peak with desired mass was detected. The crude product was triturated with ACN (15 mL) at 20° C. for 30 min. 4 (3.2 g, 5.96 mmol, 90.85% yield) was obtained as a white solid.

LCMS: RT=0.945 min, MS cal.: 536.2, [M+H]⁺=537.3

### General Procedure for Preparation of Intermediate 5:

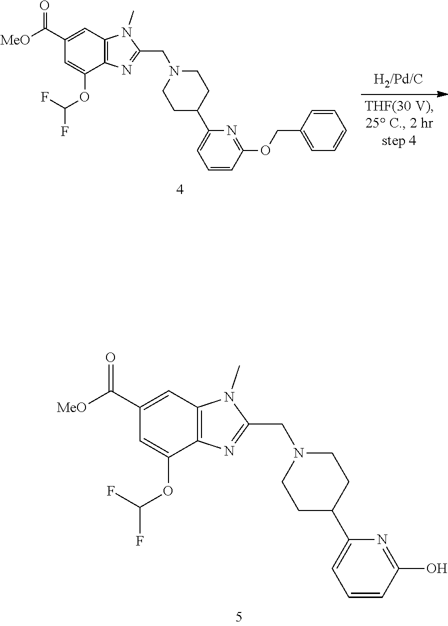 [SMILES: COC(=O)c1cc(OC(F)F)c2nc(CN3CCC(c4cccc(OCc5ccccc5)n4)CC3)n(C)c2c1 ; COC(=O)c1cc(OC(F)F)c2nc(CN3CCC(c4cccc(O)n4)CC3)n(C)c2c1]

To a mixture of 4 (3.2 g, 5.96 mmol, 1 eq) and Pd/C (507.74 mg, 477.11 μmol, 10% purity, 0.08 eq) in THF (90 mL). Then the mixture was degassed and purged with N₂ three times. Then the mixture was degassed and purged with H₂ (15 Psi) three times. Then the mixture was de-gassed under reduced pressure and recharged with H₂ (15 Psi). The mixture was stirred at 25° C. for 2 hours under H₂ (15 Psi). LC-MS showed 4 was consumed completely and one main peak with desired mass was detected. The mixture was filtered to obtain the organic phase. And then the organic phase was concentrated in vacuum. The residue was used in the next step without further purification. 5 (2.1 g, 4.70 mmol, 78.87% yield) was obtained as a white solid.

LCMS: RT=0.650 min, MS cal.: 446.2, [M+H]⁺=447.3

¹H NMR (400 MHz, CHCl3-d) δ=8.12 (d, J=1.2 Hz, 1H), 7.67 (s, 1H), 7.51 (dd, J=7.2, 9.2 Hz, 1H), 7.26 (t, J=74.4 Hz, 1H), 6.36 (d, J=8.4 Hz, 1H), 6.24 (d, J=7.2 Hz, 1H), 4.01 (s, 3H), 3.96 (s, 3H), 3.91 (s, 2H), 3.02 (br d, J=11.6 Hz, 2H), 2.53 (tt, J=3.6, 12.0 Hz, 1H), 2.30 (dt, J=2.0, 11.6 Hz, 2H), 1.95-1.88 (m, 2H), 1.74 (dq, J=3.6, 12.4 Hz, 2H)

### General Procedure for Preparation of Intermediate 6:

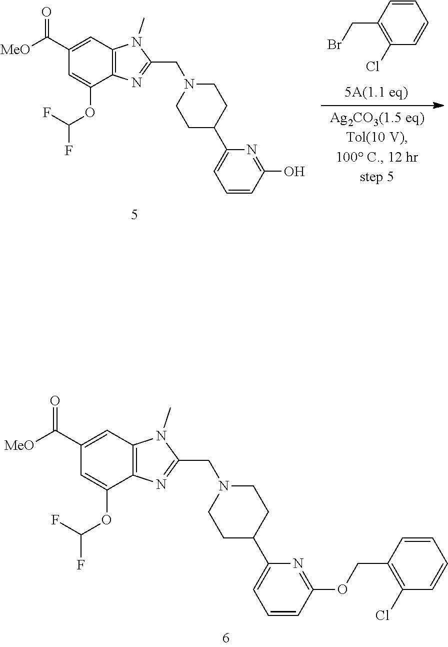 [SMILES: COC(=O)c1cc(OC(F)F)c2nc(CN3CCC(c4cccc(O)n4)CC3)n(C)c2c1 ; Clc1ccccc1CBr ; COC(=O)c1cc(OC(F)F)c2nc(CN3CCC(c4cccc(OCc5ccccc5Cl)n4)CC3)n(C)c2c1]

To a mixture of 5 (0.1 g, 223.99 μmol, 1 eq) in Toluene (1 mL) was added 5A (50.63 mg, 246.39 μmol, 31.98 μL, 1.1 eq) and Ag₂CO₃ (92.64 mg, 335.99 μmol, 15.25 μL, 1.5 eq). The mixture was stirred at 100° C. for 16 hr. LC-MS showed 5 was consumed completely and one main peak with desired mass was detected. The reaction mixture was diluted with water (10 mL) and the 5 resulting mixture was extracted with ethyl acetate (3 mL*3). The combined organic phases were washed with brine (10 mL), dried with anhydrous Na₂SO₄, filtered and the filtrate was concentrated in vacuum to afford a yellow residue. The residue was purified by prep-TLC (Petroleum ether:Ethyl acetate=10:1, product R_{f}=0.5). 6 (115 mg, 201.40 μmol, 89.91% yield) was obtained as a white solid.

LCMS: RT=0.994 min, MS cal.: 570.2, [M+H]⁺=571.3

¹H NMR (400 MHz, CHCl3-d) δ=8.13 (d, J=1.2 Hz, 1H), 7.67 (s, 1H), 7.61-7.55 (m, 1H), 7.51 (dd, J=3.6, 5.6 Hz, 1H), 7.40 (dd, J=3.6, 5.6 Hz, 1H), 7.26 (t, J=2.8 Hz, 2H), 7.27 (t, J=74.0 Hz, 1H), 6.82 (d, J=7.2 Hz, 1H), 6.66 (d, J=8.0 Hz, 1H), 5.47 (s, 2H), 4.03 (s, 3H), 3.96 (s, 3H), 3.91 (s, 2H), 3.00 (br d, J=11.2 Hz, 2H), 2.69-2.59 (m, 1H), 2.36-2.27 (m, 2H), 1.89-1.82 (m, 4H)

### General Procedure for Preparation of Compound 248 (7):

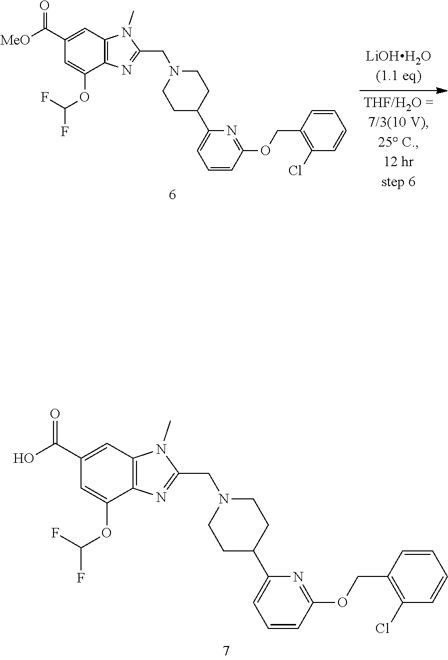 [SMILES: COC(=O)c1cc(OC(F)F)c2nc(CN3CCC(c4cccc(OCc5ccccc5Cl)n4)CC3)n(C)c2c1 ; Cn1c(CN2CCC(c3cccc(OCc4ccccc4Cl)n3)CC2)nc2c(OC(F)F)cc(C(=O)O)cc21]

To a mixture of 6 (0.1 g, 175.13 μmol, 1 eq) in THF (1.4 mL) and H₂O (0.6 mL) was added LiOH·H₂O (8.08 mg, 192.64 μmol, 1.1 eq). The mixture was stirred at 25° C. for 24 hr. LC-MS showed 6 was consumed completely and one main peak with desired mass was detected. The reaction mixture was diluted with water (6 mL) and the resulting mixture was extracted with ethyl acetate (2 mL*3). The combined organic phases were washed with brine (5 mL), dried with anhydrous Na₂SO₄, filtered and the filtrate was concentrated in vacuum to afford a yellow residue. The residue was purified by prep-HPLC (column: 2_Phenomenex Gemini C₁₈ 75*40 mm*3 um; mobile phase: [H₂O (10 mM NH₄HCO₃)-ACN]; gradient: 20%-50% B over 8.0 min). 2-((4-(6-((2-Chlorobenzyl)oxy)pyridin-2-yl)piperidin-1-yl)methyl)-4-(difluoromethoxy)-1-methyl-1H-benzo[d]imidazole-6-carboxylic acid (28.81 mg, 51.72 μmol, 29.54% yield) was obtained as a white solid.

LCMS: RT=2.750 min, MS cal.: 556.2, [M+H]⁺=557.2

HPLC: RT=11.607 min, purity: 99.03%

¹H NMR (400 MHz, DMSO-d₆) δ=8.10 (d, J=1.2 Hz, 1H), 7.68 (s, 1H), 7.59 (t, J=7.6 Hz, 1H), 7.51 (dd, J=3.6, 5.6 Hz, 1H), 7.40 (dd, J=3.6, 5.6 Hz, 1H), 7.27-7.24 (m, 2H), 7.25 (t, J=74.0 Hz, 1H), 6.83 (d, J=7.2 Hz, 1H), 6.67 (d, J=8.4 Hz, 1H), 5.47 (s, 2H), 4.02 (s, 3H), 3.98 (s, 2H), 3.08 (br d, J=11.6 Hz, 2H), 2.67 (td, J=7.6, 15.6 Hz, 1H), 2.41 (td, J=7.6, 10.8 Hz, 2H), 1.92-1.86 (m, 4H)

### Example A165—Preparation of Compound 249. 2-((4-(6-((4-Chloro-2-fluorobenzyl)oxy)pyridin-2-yl)piperidin-1-yl)methyl)-4-methoxy-1-methyl-1H-benzo[d]imidazole-6-carboxylic acid (Compound 249) (6)

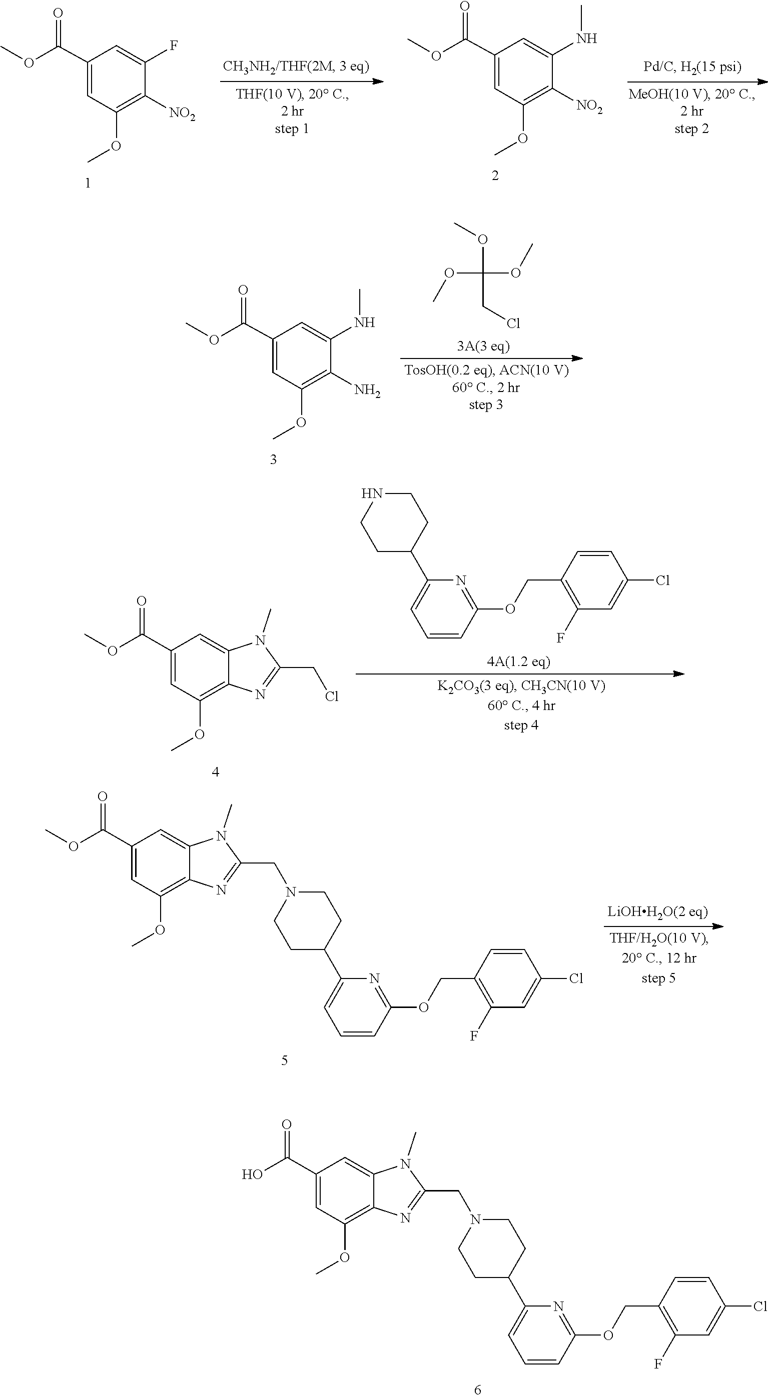

### General Procedure for Preparation of Intermediate 2:

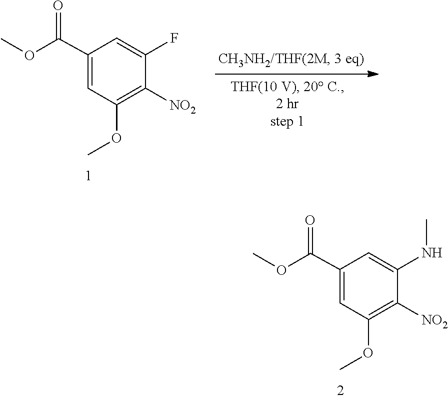 [SMILES: COC(=O)c1cc(F)c([N+](=O)[O-])c(OC)c1 ; CNc1cc(C(=O)OC)cc(OC)c1[N+](=O)[O-]]

Equip a 25 mL round bottom flask. THF (2 mL) was charged to the round bottom flask, then 1 (400 mg, 1.75 mmol, 1 eq) was added to the mixture at 20° C. Methylamine (2 M, 4.36 mL, 5 eq) was added to the reaction mixture at 20° C. under N₂ atmosphere. After the addition, the suspension was degassed and purged with N₂ for 3 times, and then the mixture was stirred under N₂ atmosphere at 20° C. for 2 hr. LC-MS showed ˜7% of 1 remained. Several new peaks were shown on LC-MS and ˜87% of desired mass was detected. The mixture quenched by addition 1 M FA 10 mL at 20° C., and then was diluted with H₂O (10 mL), extracted with EtOAc (10 mL*3). The combined organic layer was washed with 10 mL brine, dried over Na₂SO₄, filtered and the filtrate was concentrated to give a crude product. The residue was purified by column chromatography (SiO₂, Petroleum ether/Ethyl acetate=6/1). 2 (300 mg, 1.25 mmol, 72% yield) was obtained as a red solid.

LCMS: RT=0.462 min, MS cal.: 240.1, 241.1, [M+H]⁺=241.1, 242.1

¹H NMR (400 MHz, CHCl3-d) δ=7.08 (d, J=1.6 Hz, 1H), 6.94 (d, J=1.6 Hz, 1H), 3.95 (d, J=3.6 Hz, 6H), 2.97 (s, 3H)

### General Procedure for Preparation of Intermediate 3:

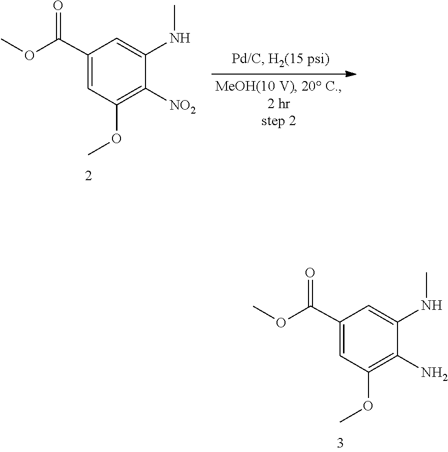

Equip a 10 mL round bottom flask. MeOH (2 mL) was charged to the round bottom flask, then Pd/C (100.00 mg) was added to the mixture at 20° C., 2 (200 mg, 832.60 μmol, 1 eq) in MeOH (2 mL) was added to the reaction mixture at 20° C. under N₂ atmosphere. After the addition, the suspension was degassed and purged with H₂ for 3 times, and then the mixture was stirred under H₂ (15 Psi) atmosphere at 20° C. for 2 hr. TLC indicated 2 was consumed completely and one new spot formed. The reaction was clean according to TLC. The suspension was filtered with celite, and the filter cake was washed with EtOAc (15 mL). The combined filtrates were concentrated to dryness. The residue was purified by column chromatography (SiO₂, Petroleum ether/Ethyl acetate=5/1). 3 (140 mg, 665.94 μmol, 80% yield) was obtained as a white solid.

¹H NMR (400 MHz, CHCl3-d) δ=7.19 (d, J=1.2 Hz, 1H), 7.14 (d, J=1.2 Hz, 1H), 5.31 (s, 1H), 3.90 (d, J=6.88 Hz, 6H), 3.16-3.68 (m, 2H), 2.92 (s, 3H)

### General Procedure for Preparation of Intermediate 4:

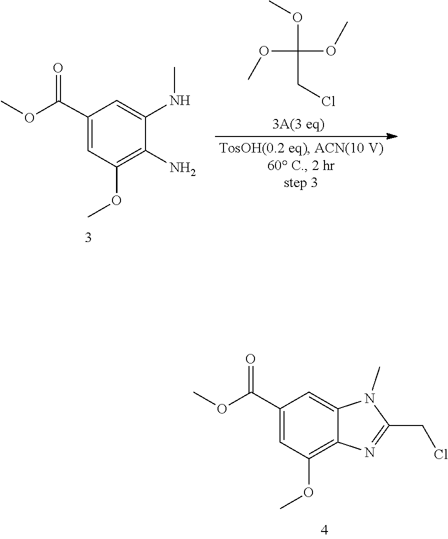

To a solution of 3 (180 mg, 856.21 μmol, 1 eq), 2-chloro-1,1,1-trimethoxy-ethane (397.09 mg, 2.57 mmol, 346.20 μL, 3 eq) in ACN (2 mL) was added TosOH (29.49 mg, 171.24 μmol, 0.2 eq). The mixture was stirred at 60° C. for 2 hr. LC-MS showed 3 was consumed completely and the desired mass was detected as a main peak. The mixture was diluted with H₂O (5 mL), extracted with EtOAc (5 mL*3). The combined organic layer was washed with 10 mL brine, dried over Na₂SO₄, filtered and the filtrate was concentrated to give a crude product. The residue was purified by column chromatography (SiO₂, Petroleum ether/Ethyl acetate=10/1). 4 (180 mg, 669.90 μmol, 78% yield) was obtained as a white solid.

LCMS: RT=0.381 min, MS cal.: 268.1, 270.1, [M+H]⁺=269.0, 271.0

¹H NMR (400 MHz, CHCl3-d) δ=7.78 (s, 1H), 7.41 (s, 1H), 4.87 (s, 2H), 4.07 (s, 3H), 3.97 (s, 3H), 3.92 (s, 3H)

### General Procedure for Preparation of Intermediate 5:

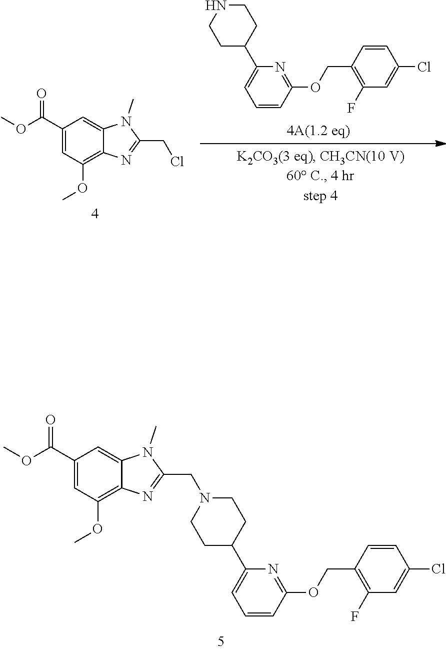 [SMILES: COC(=O)c1cc(OC)c2nc(CCl)n(C)c2c1 ; Fc1cc(Cl)ccc1COc1cccc(C2CCNCC2)n1 ; COC(=O)c1cc(OC)c2nc(CN3CCC(c4cccc(OCc5ccc(Cl)cc5F)n4)CC3)n(C)c2c1]

To a solution of 4 (100 mg, 372.17 μmol, 1 eq), 4A (143.26 mg, 446.60 μmol, 1.2 eq) in ACN (1 mL) was added K₂CO₃ (154.31 mg, 1.12 mmol, 3 eq). The mixture was stirred at 60° C. for 4 hr. LC-MS showed 4 was consumed completely and the desired mass was detected as a main peak. The mixture was diluted with H₂O (15 mL), extracted with EtOAc (15 mL*3). The combined organic layer was washed with 10 mL brine, dried over Na₂SO₄, filtered and the filtrate was concentrated to give a crude product. The residue was purified by column chromatography (SiO₂, Petroleum ether/Ethyl acetate=1/1). 5 (85 mg, 153.70 μmol, 41% yield) was obtained as a white solid.

LCMS: RT=0.470 min, MS cal.: 552.2, 554.2, [M+H]⁺=553.2, 555.2

¹H NMR (400 MHz, CHCl3-d) δ=7.79 (d, J=1.2 Hz, 1H), 7.50 (t, J=7.6 Hz, 1H), 7.44 (t, J=8.4 Hz, 1H), 7.39 (d, J=1.2 Hz, 1H), 7.13-7.07 (m, 2H), 6.73 (d, J=7.2 Hz, 1H), 6.60 (d, J=8.0 Hz, 1H), 5.41 (s, 2H), 4.07 (s, 3H), 3.98 (s, 3H), 4.07 (s, 3H), 3.89 (s, 2H), 3.07-2.89 (m, 2H), 2.69-2.53 (m, 1H), 2.37-2.20 (m, 2H), 1.94-1.75 (m, 4H)

### General Procedure for Preparation of Compound 249 (6):

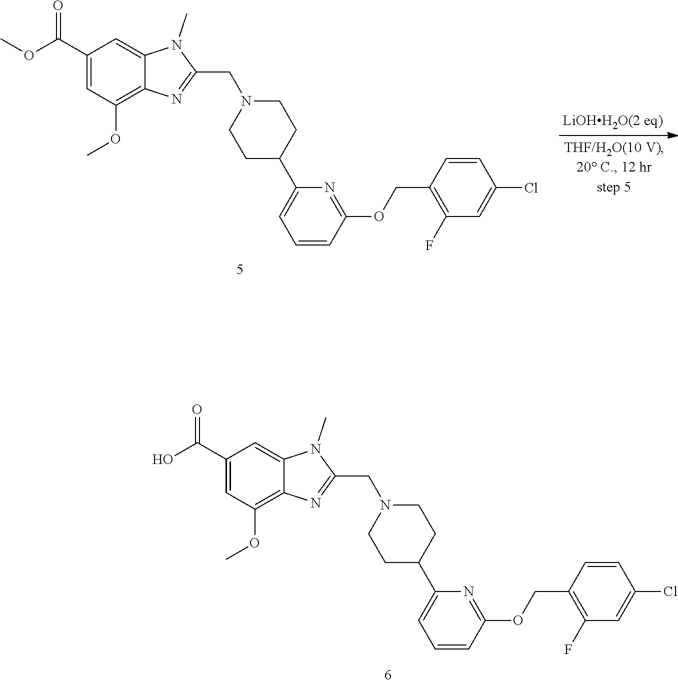 [SMILES: COC(=O)c1cc(OC)c2nc(CN3CCC(c4cccc(OCc5ccc(Cl)cc5F)n4)CC3)n(C)c2c1 ; COc1cc(C(=O)O)cc2c1nc(CN1CCC(c3cccc(OCc4ccc(Cl)cc4F)n3)CC1)n2C]

To a solution of 5 (65 mg, 117.54 μmol, 1 eq) in THF (0.7 mL) was added LiOH·H₂O (9.86 mg, 235.07 μmol, 2 eq) in H₂O (0.3 mL). The mixture was stirred at 20° C. for 12 hr. LC-MS showed 5 was consumed completely and one main peak with desired mass was detected. The mixture was purified directly. The residue was purified by prep-HPLC (column: Waters Xbridge Prep OBD C 18 150*40 mm*10 um; mobile phase: [H₂O (10 mM NH₄HCO₃)-ACN]; gradient: 25%-60% B over 8.0 min). 2-((4-(6-((4-Chloro-2-fluorobenzyl)oxy)pyridin-2-yl)piperidin-1-yl)methyl)-4-methoxy-1-methyl-1H-benzo[d]imidazole-6-carboxylic acid (26.59 mg, 49.33 μmol, 42% yield) was obtained as a white solid.

LCMS: RT=2.667 min, MS cal.: 538.2, 540.2, [M+H]⁺=539.2, 541.2

¹H NMR (400 MHz, DMSO-d6) δ=7.77 (s, 1H), 7.62 (t, J=7.6 Hz, 1H), 7.55 (t, J=8.0 Hz, 1H), 7.45 (dd, J=10.4, 2.0 Hz, 1H), 7.31-7.25 (m, 2H), 6.86 (d, J=7.2 Hz, 1H), 6.66 (d, J=8.0 Hz, 1H), 5.36 (s, 2H), 3.95 (s, 3H), 3.90 (s, 3H), 3.80 (s, 2H), 2.92 (d, J=11.2 Hz, 2H), 2.61-2.57 (m, 1H), 2.19 (t, J=10.4 Hz, 2H), 1.82-1.64 (m, 4H)

### Example A166—Preparation of Compound 250. 2-((4-(6-((2,4-Difluorobenzyl)oxy)pyridin-2-yl)piperidin-1-yl)methyl)-4-(difluoromethoxy)-1-methyl-1H-benzo[d]imidazole-6-carboxylic acid (Compound 250) (3)

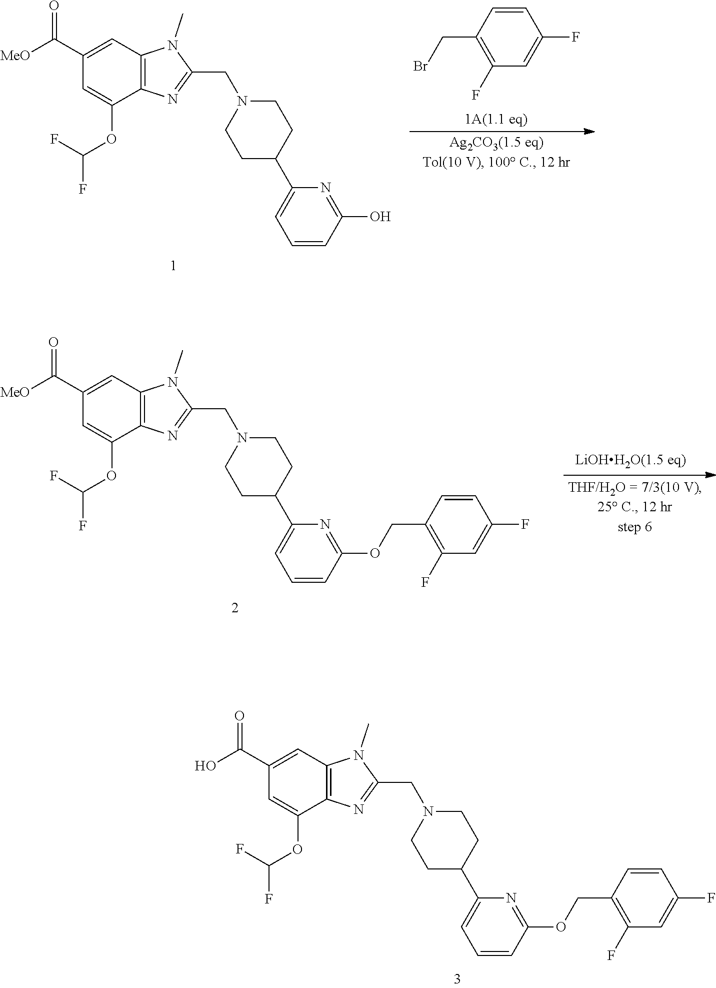 [SMILES: COC(=O)c1cc(OC(F)F)c2nc(CN3CCC(c4cccc(O)n4)CC3)n(C)c2c1 ; Fc1ccc(CBr)c(F)c1 ; COC(=O)c1cc(OC(F)F)c2nc(CN3CCC(c4cccc(OCc5ccc(F)cc5F)n4)CC3)n(C)c2c1 ; Cn1c(CN2CCC(c3cccc(OCc4ccc(F)cc4F)n3)CC2)nc2c(OC(F)F)cc(C(=O)O)cc21]

### General Procedure for Preparation of Intermediate 2:

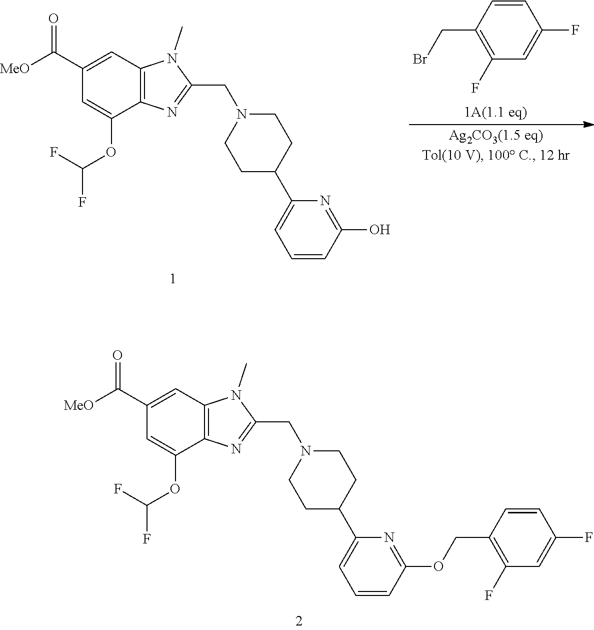 [SMILES: COC(=O)c1cc(OC(F)F)c2nc(CN3CCC(c4cccc(O)n4)CC3)n(C)c2c1 ; Fc1ccc(CBr)c(F)c1 ; COC(=O)c1cc(OC(F)F)c2nc(CN3CCC(c4cccc(OCc5ccc(F)cc5F)n4)CC3)n(C)c2c1]

To a solution of 1 (100 mg, 223.99 μmol, 1 eq) in toluene (1 mL) was added Ag₂CO₃ (92.65 mg, 335.99 μmol, 15.25 μL, 1.5 eq) and 1A (51.01 mg, 246.39 μmol, 1.1 eq). The mixture was stirred at 100° C. for 12 hr. LCMS showed 1 was consumed completely and one main peak with desired mass was detected. The reaction mixture was filtered, and the filter cake was washed with EtOAc (5 mL). The filtrate was diluted with H₂O 5 mL and extracted with EtOAc 10 mL (5 mL*2). The combined organic layers were washed with brine 5 mL, dried over Na₂SO₄, filtered, and concentrated under reduced pressure to give a residue. The residue was purified by column chromatography (SiO₂, PE/EtOAc=1/0 to 1/1). 2 (60 mg, 99.55 μmol, 44.45% yield) was obtained as a yellow oil.

LCMS: RT=1.638 min, MS cal.: 572.2/573.2, [M+H]⁺=573.0/574.0

### General Procedure for Preparation of Compound 250 (3):

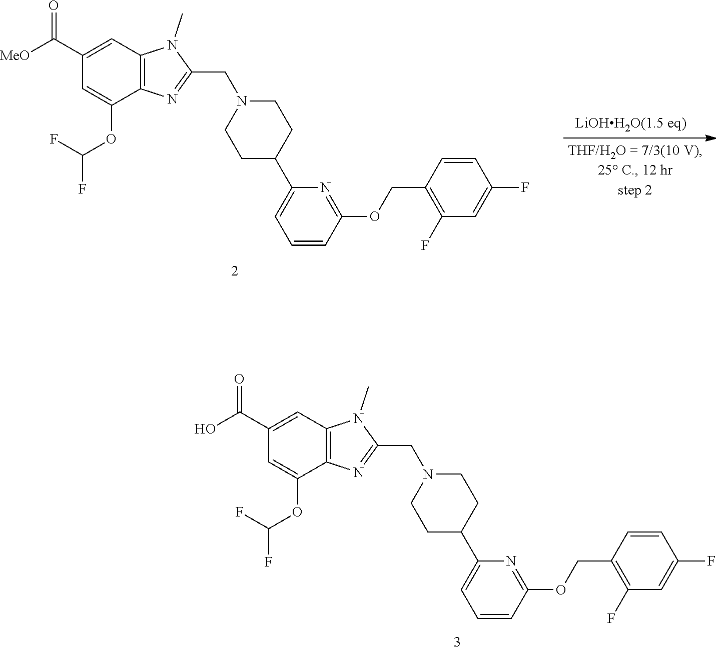 [SMILES: COC(=O)c1cc(OC(F)F)c2nc(CN3CCC(c4cccc(OCc5ccc(F)cc5F)n4)CC3)n(C)c2c1 ; Cn1c(CN2CCC(c3cccc(OCc4ccc(F)cc4F)n3)CC2)nc2c(OC(F)F)cc(C(=O)O)cc21]

To a solution of 2 (50 mg, 87.33 μmol, 1 eq) in THF (0.7 mL) and H₂O (0.3 mL) was added LiOH·H₂O (5.50 mg, 130.99 μmol, 1.5 eq). The mixture was stirred at 25° C. for 12 hr. LCMS showed 5% of 2 remained. Several new peaks were shown on LCMS and 89% of desired Compound was detected. The mixture was added THF 0.2 mL. The residue was purified by prep-HPLC (neutral condition: column: Waters Xbridge Prep OBD C18 150 40 mm*10 um; mobile phase: [H₂O (10 mM NH₄HCO₃)-ACN]; gradient: 20%-70% B over 8.0 min). 2-((4-(6-((2,4-Difluorobenzyl)oxy)pyridin-2-yl)piperidin-1-yl)methyl)-4-(difluoromethoxy)-1-methyl-1H-benzo[d]imidazole-6-carboxylic acid (22.57 mg, 38.79 μmol, 44.42% total yield) was obtained as a white solid.

LCMS: RT=2.703 min, MS cal.: 558.2/559.2, [M+H]⁺=559.2/560.2

HPLC: RT=11.321 min

¹H NMR (400 MHz, MeOH-d4) δ=8.11 (s, 1H), 7.69 (s, 1H), 7.60-7.50 (m, 2H), 7.25 (t, J=74.4 Hz 1H), 6.99-6.90 (m, 2H), 6.84-6.81 (m, 1H), 6.64-6.60 (m, 1H), 5.39 (s, 2H), 4.03 (s, 3H), 3.99 (s, 2H), 3.10 (br d, J=11.6 Hz, 2H), 2.73-2.63 (m, 1H), 2.47-2.37 (m, 2H), 1.95-1.87 (m, 4H)

### Example A167—Preparation of Compound 251. 4-(Difluoromethoxy)-2-((4-(6-((2-fluorobenzyl)oxy)pyridin-2-yl)piperidin-1-yl)methyl)-1-methyl-1H-benzo[d]imidazole-6-carboxylic acid (Compound 251) (3)

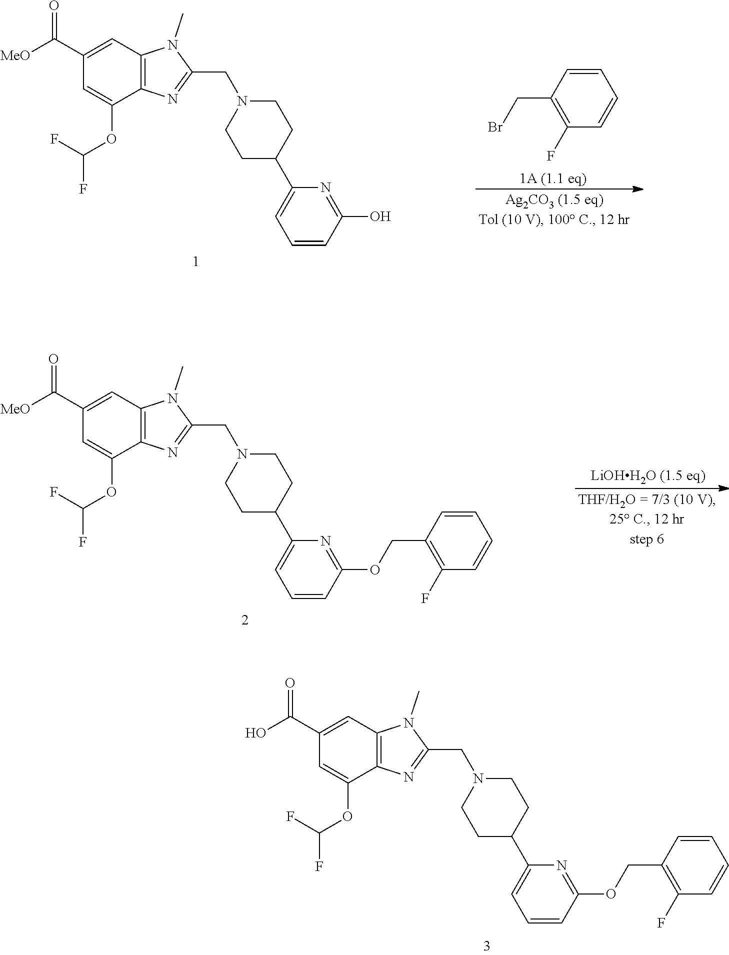 [SMILES: COC(=O)c1cc(OC(F)F)c2nc(CN3CCC(c4cccc(O)n4)CC3)n(C)c2c1 ; Fc1ccccc1CBr ; COC(=O)c1cc(OC(F)F)c2nc(CN3CCC(c4cccc(OCc5ccccc5F)n4)CC3)n(C)c2c1 ; Cn1c(CN2CCC(c3cccc(OCc4ccccc4F)n3)CC2)nc2c(OC(F)F)cc(C(=O)O)cc21]

### General Procedure for Preparation of Intermediate 2:

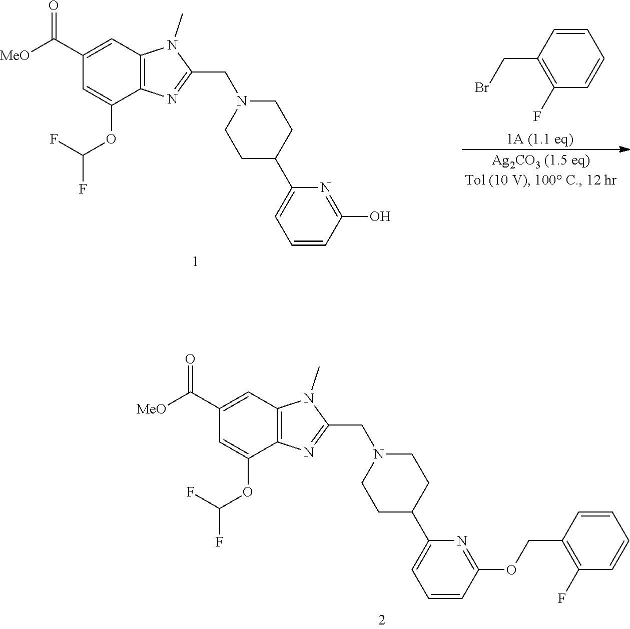 [SMILES: COC(=O)c1cc(OC(F)F)c2nc(CN3CCC(c4cccc(O)n4)CC3)n(C)c2c1 ; Fc1ccccc1CBr ; COC(=O)c1cc(OC(F)F)c2nc(CN3CCC(c4cccc(OCc5ccccc5F)n4)CC3)n(C)c2c1]

To a solution of 1 (100 mg, 223.99 μmol, 1 eq) in toluene (1 mL) was added Ag₂CO₃ (92.65 mg, 335.99 μmol, 15.25 μL, 1.5 eq) and 1A (46.57 mg, 246.39 μmol, 29.72 μL, 1.1 eq). The mixture was stirred at 100° C. for 2 hr. After monitoring, the reaction was not completely. The mixture was added 1A (12.70 mg, 67.20 μmol, 8.11 μL, 0.3 eq). The mixture was stirred at 100° C. for 12 hr. LCMS showed 1 was consumed completely and one main peak with desired mass was detected. The reaction mixture was filtered, and the filter cake was washed with EtOAc (2 mL). The filtrate was diluted with H₂O 2 mL and extracted with EtOAc 4 mL (2 mL*2). The combined organic layers were washed with brine 4 mL, dried over Na₂SO₄, filtered and concentrated under reduced pressure to give a residue. The residue was purified by column chromatography (SiO₂, PE/EtOAc=1/0 to 1/1). 2 (60 mg, 108.19 μmol, 48.30% yield) was obtained as a white solid.

LCMS: RT=1.625 min, MS cal.: 554.2/555.2, [M+H]⁺=555.0/556.0

### General Procedure for Preparation of Compound 251 (3):

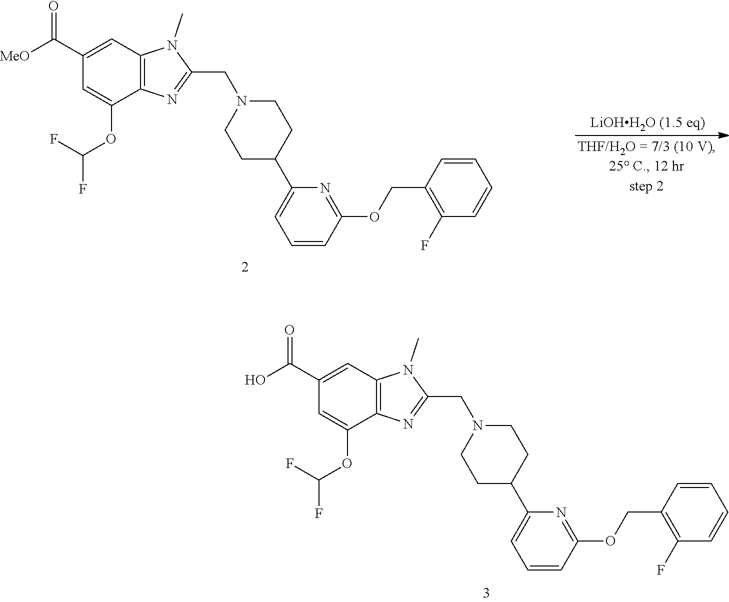 [SMILES: COC(=O)c1cc(OC(F)F)c2nc(CN3CCC(c4cccc(OCc5ccccc5F)n4)CC3)n(C)c2c1 ; Cn1c(CN2CCC(c3cccc(OCc4ccccc4F)n3)CC2)nc2c(OC(F)F)cc(C(=O)O)cc21]

To a solution of 2 (50 mg, 90.16 μmol, 1 eq) in THF (0.7 mL) and H₂O (0.3 mL) was added

LiOH·H₂O (5.68 mg, 135.24 μmol, 1.5 eq). The mixture was stirred at 25° C. for 12 hr. LCMS showed 2 was consumed completely and one main peak with desired mass was detected. The mixture was added THF 0.2 mL. The residue was purified by prep-HPLC (neutral condition: column: Waters Xbridge Prep OBD C18 150*40 mm*10 um; mobile phase: [H₂O (10 mM NH₄HCO₃)-ACN]; gradient: 15%-65% B over 8.0 min). 4-(Difluoromethoxy)-2-((4-(6-((2-fluorobenzyl)oxy)pyridin-2-yl)piperidin-1-yl)methyl)-1-methyl-1H-benzo[d]imidazole-6-carboxylic acid (24.79 mg, 44.03 μmol, 48.83% total yield) was obtained as a white solid.

LCMS: RT=2.687 min, MS cal.: 540.2/541.2, [M+H]⁺=541.2/542.2

HPLC: RT=11.082 min

¹H NMR (400 MHz, MeOH-d4) δ=8.11 (s, 1H), 7.69 (s, 1H), 7.57 (t, J=8.0 Hz, 1H), 7.53-7.46 (m, 1H), 7.35-7.28 (m, 1H), 7.25 (t, J=74.4 Hz, 1H), 7.19-7.03 (m, 2H), 6.82 (d, J=7.2 Hz, 1H), 6.63 (d, J=8.0 Hz, 1H), 5.43 (s, 2H), 4.03 (s, 3H), 3.99 (br s, 2H), 3.15-3.06 (m, 2H), 2.74-2.63 (m, 1H), 2.47-2.37 (m, 2H), 1.99-1.87 (m, 4H)

### Example A168—Preparation of Compound 252. 2-((4-(6-((4-Chlorobenzyl)oxy)pyridin-2-yl)piperidin-1-yl)methyl)-4-(difluoromethoxy)-1-methyl-1H-benzo[d]imidazole-6-carboxylic acid (Compound 252) (3)

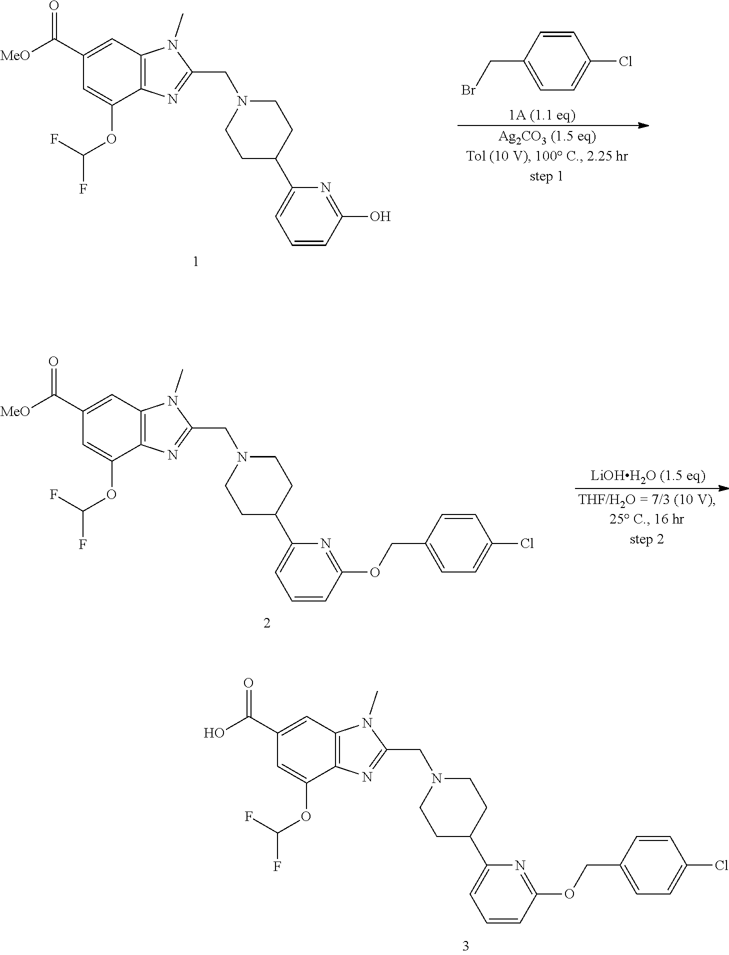 [SMILES: COC(=O)c1cc(OC(F)F)c2nc(CN3CCC(c4cccc(O)n4)CC3)n(C)c2c1 ; Clc1ccc(CBr)cc1 ; COC(=O)c1cc(OC(F)F)c2nc(CN3CCC(c4cccc(OCc5ccc(Cl)cc5)n4)CC3)n(C)c2c1 ; Cn1c(CN2CCC(c3cccc(OCc4ccc(Cl)cc4)n3)CC2)nc2c(OC(F)F)cc(C(=O)O)cc21]

### General Procedure for Preparation of Intermediate 2:

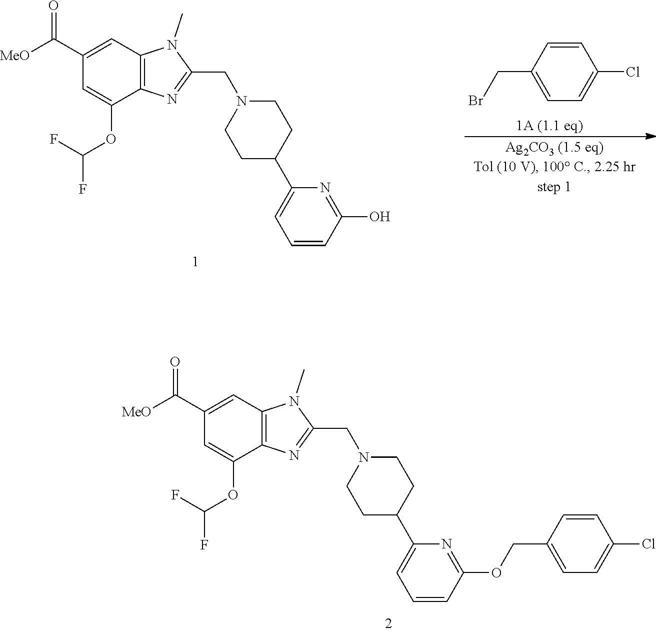 [SMILES: COC(=O)c1cc(OC(F)F)c2nc(CN3CCC(c4cccc(O)n4)CC3)n(C)c2c1 ; Clc1ccc(CBr)cc1 ; COC(=O)c1cc(OC(F)F)c2nc(CN3CCC(c4cccc(OCc5ccc(Cl)cc5)n4)CC3)n(C)c2c1]

A mixture of 1 (0.1 g, 223.99 μmol, 1 eq), 1A (50.63 mg, 246.39 μmol, 1.1 eq) in toluene (4 mL) was degassed and purged with N₂ for 3 times, then Ag₂CO₃ (92.65 mg, 335.99 μmol, 15.25 μL, 1.5 eq) was added to the mixture. The mixture was stirred at 100° C. for 2.25 hr under N₂ atmosphere. LCMS showed 1 was consumed completely and one main peak with desired mass was detected. The reaction mixture was filtered and concentrated under reduced pressure to give a residue. The residue was purified by column chromatography (SiO₂, Petroleum ether/Ethyl acetate=20/1 to 3/1). 2 (0.1 g, 163.74 μmol, 73.10% yield, 93.5% purity) was obtained as a white solid.

LCMS: RT=1.698 min, MS cal.: 570.18, [M+H]⁺=571.0

### General Procedure for Preparation of Compound 252 (3):

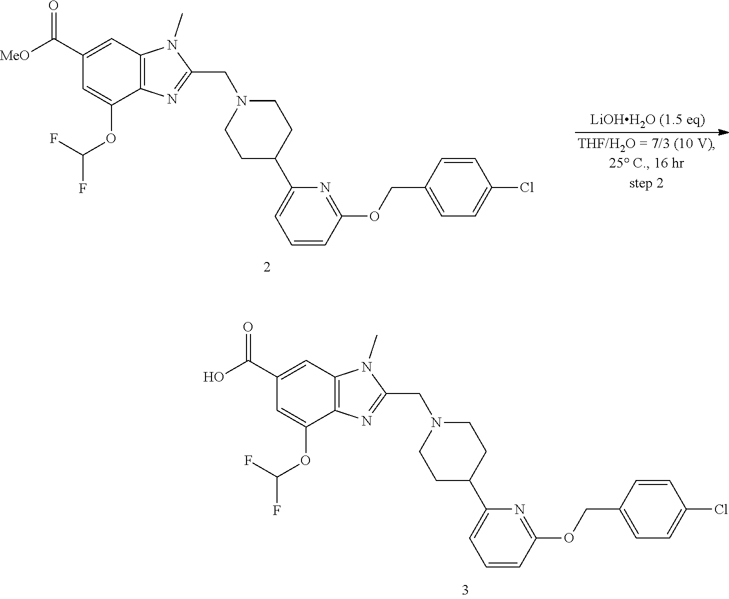 [SMILES: COC(=O)c1cc(OC(F)F)c2nc(CN3CCC(c4cccc(OCc5ccc(Cl)cc5)n4)CC3)n(C)c2c1 ; Cn1c(CN2CCC(c3cccc(OCc4ccc(Cl)cc4)n3)CC2)nc2c(OC(F)F)cc(C(=O)O)cc21]

To a solution of 2 (96 mg, 168.12 μmol, 1 eq) in THF (0.7 mL) was added LiOH H₂O (10.58 mg, 252.18 μmol, 1.5 eq) in H₂O (0.3 mL) at 25° C. The mixture was stirred at 25° C. for 16 hr. LCMS showed 2 was consumed completely and one main peak with desired m/z was detected. The reaction mixture was concentrated under reduced pressure to remove THF. The residue was purified by prep-HPLC (column: Waters Xbridge Prep OBD C₁₈ 150*40 mm*10 um; mobile phase: [H₂O (10 mM NH₄HCO₃)-ACN]; gradient: 30%-60% B over 8.0 min). 2-((4-(6-((4-Chlorobenzyl)oxy)pyridin-2-yl)piperidin-1-yl)methyl)-4-(difluoromethoxy)-1-methyl-1H-benzo[d]imidazole-6-carboxylic acid (29.76 mg, 52.90 μmol, 31.46% yield, 99% purity) was obtained as a white solid.

LCMS: RT=1.688 min, MS cal.: 556.17, [M+H]⁺=557.2

HPLC: RT=11.837 min, purity: 99%

¹H NMR (400 MHz, MeOH-d4) δ=8.10 (d, J=1.2 Hz, 1H), 7.68 (s, 1H), 7.57 (dd, J=8.8 Hz, 1H), 7.45-7.39 (m, 2H), 7.36-7.30 (m, 2H), 7.25 (t, J=76.0 Hz, 1H), 6.81 (d, J=7.2 Hz, 1H), 6.63 (d, J=8.0 Hz, 1H), 5.35 (s, 2H), 4.02 (s, 3H), 3.98 (s, 2H), 3.10 (br d, J=11.6 Hz, 2H), 2.72-2.62 (m, 1H), 2.47-2.37 (m, 2H), 1.97-1.85 (m, 4H).

### Example A169—Preparation of Compound 253. 2-((4-(6-((4-Chloro-2-fluorobenzyl)oxy)pyridin-2-yl)piperidin-1-yl)methyl)-4-(difluoromethoxy)-1-methyl-1H-imidazo[4,5-c]pyridine-6-carboxylic acid (Compound 253) (9)

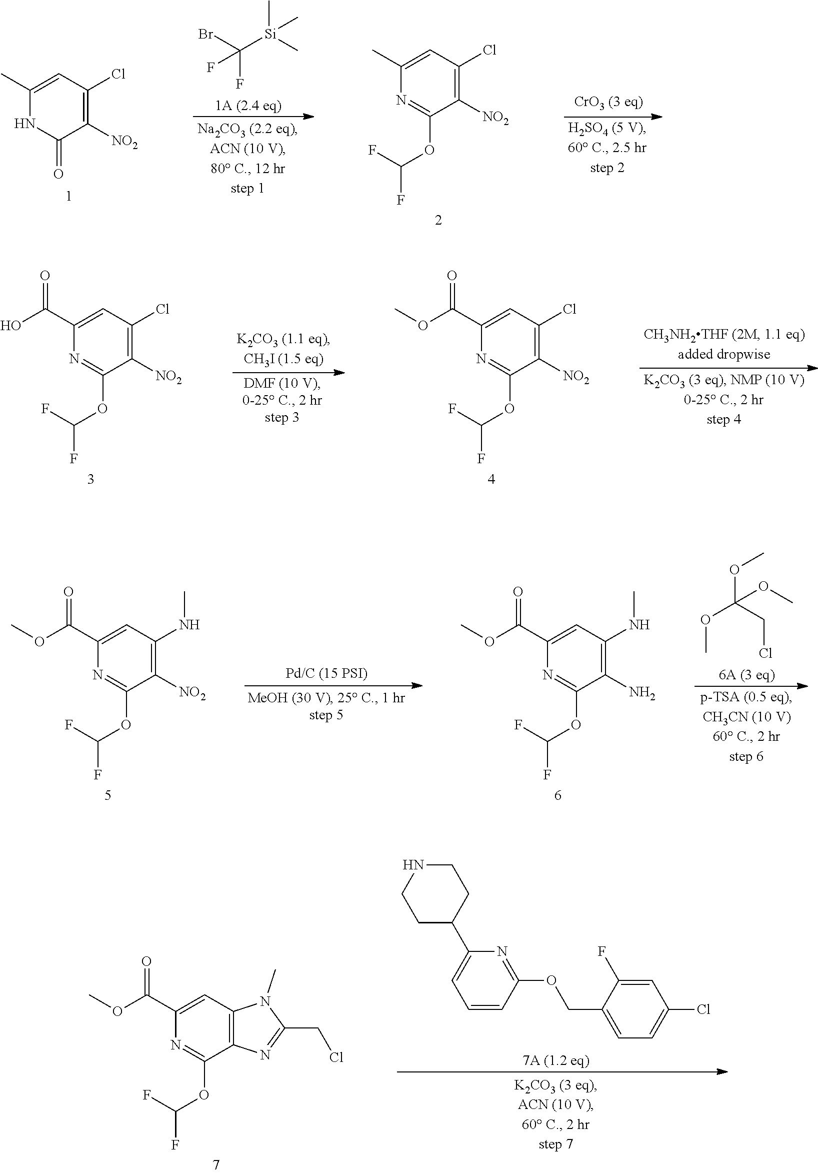

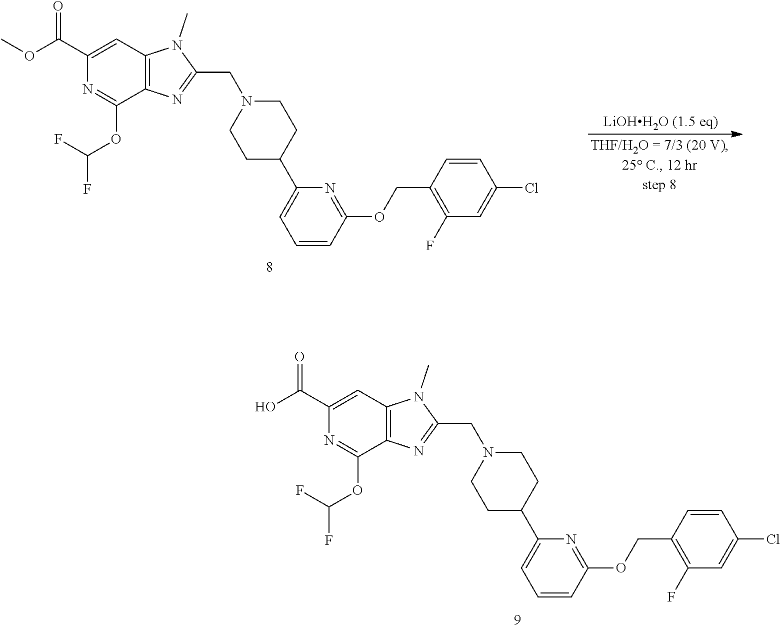 [SMILES: COC(=O)c1cc2c(nc(CN3CCC(c4cccc(OCc5ccc(Cl)cc5F)n4)CC3)n2C)c(OC(F)F)n1 ; Cn1c(CN2CCC(c3cccc(OCc4ccc(Cl)cc4F)n3)CC2)nc2c(OC(F)F)nc(C(=O)O)cc21]

### General Procedure for Preparation of Intermediate 2:

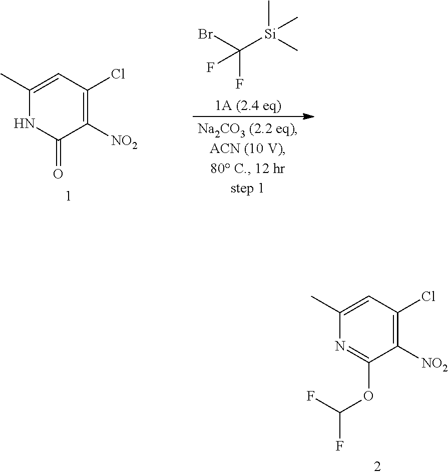

2 reactions were carried out in parallel.

A mixture of 1 (1 g, 5.30 mmol, 1 eq), 1A (2.58 g, 12.73 mmol, 2.4 eq), Na₂CO₃ (968.33 mg, 11.67 mmol, 2.2 eq) in ACN (10 mL) was degassed and purged with N₂ for 3 times, and then the mixture was stirred at 80° C. for 12 hr under N₂ atmosphere. TLC (Petroleum ether:Ethyl acetate=3:1, R_{f}=0.61) indicated ˜10% of 1 was remained, and one major new spot with lower polarity was detected. After 12 hr, the 2 reactions were combined for workup, the reaction mixture was diluted with H₂O (30 mL) and extracted with EtOAc (50 mL*3). The combined organic layers were washed with brine (10 mL*2), dried over Na₂SO₄, filtered, and concentrated under reduced pressure to give a residue. The residue was purified by column chromatography (SiO₂, Petroleum ether/Ethyl acetate=100/1 to 3/1). 2 (850 mg, 3.56 mmol, 33.59% yield) was obtained as a yellow oil.

¹H NMR (400 MHz, CHCl3-d) δ=7.67-7.29 (m, 1H), 7.13 (s, 1H), 2.54 (s, 3H)

### General Procedure for Preparation of Intermediate 3:

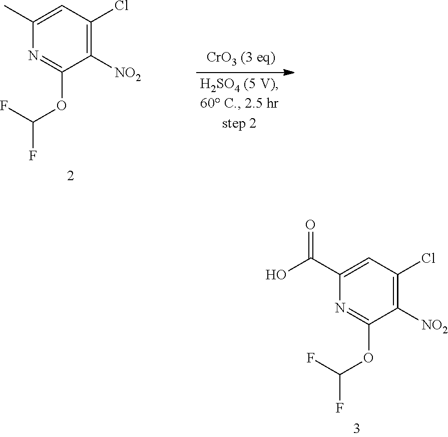 [SMILES: Cc1cc(Cl)c([N+](=O)[O-])c(OC(F)F)n1 ; O=C(O)c1cc(Cl)c([N+](=O)[O-])c(OC(F)F)n1]

Equip a 25 mL three-necked round bottom flask, and thermometer, N₂ balloon. conc. H₂SO₄ (3.5 mL) was charged to the 25 mL three-necked round bottom flask, then 2 (700 mg, 2.93 mmol, 1 eq) was added at 25° C. under N₂. At 60° C. (inner temperature), CrO₃ (880.17 mg, 8.80 mmol, 3 eq) was added in portions to the reaction mixture at 60° C. After addition, the mixture was stirred at 60° C. for 2.5 hr. The mixture was monitored by TLC. TLC (Petroleum ether:Ethyl acetate=0:1, R_{f}=0.52) indicated 2 was consumed, and one major new spot with larger polarity was detected. The reaction mixture was cooled to 25° C. and then poured into (20 mL) ice water. The reaction mixture was extracted with EtOAc (20 mL*3). The combined organic layers were washed with brine (10 mL), dried over Na₂SO₄, filtered, and concentrated under reduced pressure to give a residue. 3 (670 mg, crude) was obtained as a green solid.

¹H NMR (400 MHz, DMSO-d₆) δ=8.26 (s, 1H), 8.01-7.61 (m, 1H)

### General Procedure for Preparation of Intermediate 4:

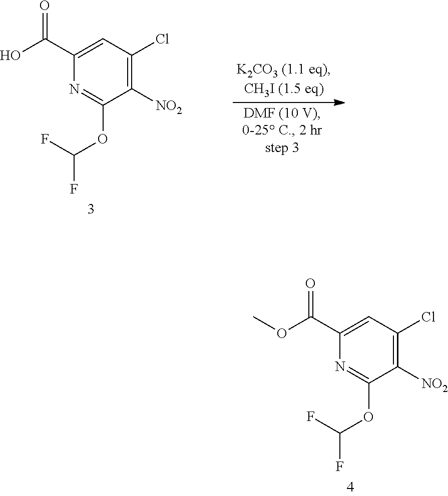 [SMILES: O=C(O)c1cc(Cl)c([N+](=O)[O-])c(OC(F)F)n1 ; COC(=O)c1cc(Cl)c([N+](=O)[O-])c(OC(F)F)n1]

Equip a 50 mL three-necked round bottom flask, thermometer, N₂ balloon. DMF (5 mL) was charged to the three-necked round bottom flask, then 3 (500 mg, 1.86 mmol, 1 eq) and K₂CO₃ (283.04 mg, 2.05 mmol, 1.1 eq) was added to the mixture at 25° C. At 0° C. inner temperature, CH₃I (396.39 mg, 2.79 mmol, 173.85 μL, 1.5 eq) was added to the reaction mixture at 0° C. After the addition, the mixture was stirred at 25° C. for 2 hr. The reaction was monitored by TLC. TLC (Petroleum ether:Ethyl acetate=0:1, R_{f}=0.51) indicated 3 was consumed, and one major new spot with lower polarity was detected. After 2 hr, the reaction mixture was added to H₂O 15 mL at 0° C. The mixture was extracted by EtOAc (20 mL). Then organic phase was combined and washed by brine (10 mL), dried with Na₂SO₄, filtered. The organic phase was concentrated under reduced pressure at 40° C. to give a residue. The residue was purified by column chromatography

(SiO₂, Petroleum ether/Ethyl acetate=100/1 to 3/1). 4 (450 mg, 1.59 mmol, 85.53% yield) was obtained as a yellow oil.

¹H NMR (400 MHz, CHCl3-d) δ=8.08 (s, 1H), 7.78-7.41 (m, 1H), 4.02 (s, 3H)

### General Procedure for Preparation of Intermediate 5:

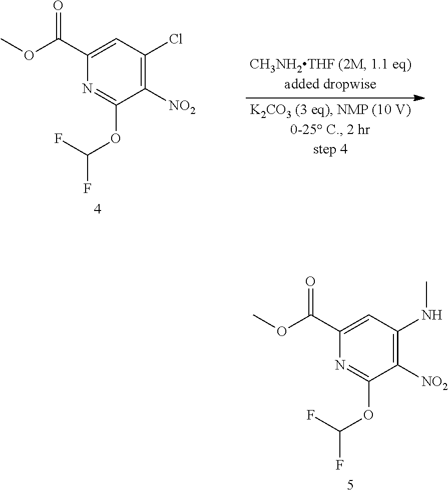 [SMILES: COC(=O)c1cc(Cl)c([N+](=O)[O-])c(OC(F)F)n1 ; CNc1cc(C(=O)OC)nc(OC(F)F)c1[N+](=O)[O-]]

Equip a 25 mL three-necked bottom flask, and N₂ balloon.

NMP (3.5 mL) was charged to the 25 mL three-necked round bottom flask, 4 (350 mg, 1.24 mmol, 1 eq) and K₂CO₃ (513.53 mg, 3.72 mmol, 530.81 μL, 3 eq) was added at 25° C. At 0° C. (inner temperature), CH₃NH₂·THF (2 M, 309.64 μL, 0.5 eq) was added to the reaction mixture. After the addition, the mixture was stirred at 0° C. for 1 hr. At 0° C. (inner temperature), CH₃NH₂·THF (2 M, 371.57 μL, 0.6 eq) was added to the reaction mixture. After the addition, the mixture was stirred at 0° C. for 1 hr. The mixture was monitored by TLC. TLC (Petroleum ether:Ethyl acetate=5:1, R_{f}=0.17) indicated ˜20% of 4 remained, and one major new spot with larger polarity was detected. The reaction mixture was added FA (1 M) to pH=5 at 25° C. The mixture was extracted by EtOAc (10 mL*3). Then organic phase was combined and washed by brine (10 mL), dried with Na₂SO₄, filtered. The organic phase was concentrated under reduced pressure at 45° C. to give a residue. The residue was purified by column chromatography (SiO₂, Petroleum ether/Ethyl acetate=100/1 to 3/1). 5 (160 mg, 577.24 μmol, 46.61% yield) was obtained as a yellow solid.

LCMS: RT=0.699 min, MS cal.: 277.2, [M−H]⁻=276.1

¹H NMR (400 MHz, DMSO-d₆) δ=8.00 (br d, J=4.4 Hz, 1H), 7.90-7.50 (m, 1H), 7.33 (s, 1H), 3.87 (s, 3H), 2.93 (d, J=4.4 Hz, 3H)

### General Procedure for Preparation of Intermediate 6:

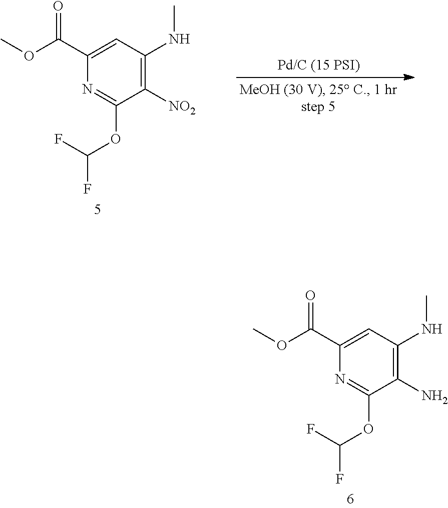

Equip a 50 mL round bottom flask, H₂ (15 psi) atmosphere. MeOH (2 mL) was charged to the 50 mL round bottom flask, then 5 (140 mg, 505.08 μmol, 1 eq) was added to the mixture at 25° C. Pd/C (53.75 mg) in MeOH (2 mL) was added to the reaction mixture at 25° C. under Ar atmosphere. After the addition, the suspension was degassed and purged with H₂ for 3 times, and then the mixture was stirred under H₂ atmosphere at 25° C. for 1 hr. The mixture was monitored by TLC and LCMS. LCMS (product: RT=0.615 min) showed the 5 was consumed completely. The reaction mixture was diluted with MeOH (15 mL), and then filtered through celite pad. The filter cake was rinsed with MeOH (15 mL*3), and the filtrate was concentrated. 6 (124.86 mg, crude) was obtained as a yellow solid.

LCMS: RT=0.608 min, MS cal.: 247.2, [M−H]⁻=246.1

¹H NMR (400 MHz, DMSO-d₆) δ=7.79-7.32 (m, 1H), 7.05 (s, 1H), 5.98 (br d, J=4.8 Hz, 1H), 5.15 (s, 2H), 3.78 (s, 3H), 2.82 (d, J=4.8 Hz, 3H)

### General Procedure for Preparation of Intermediate 7:

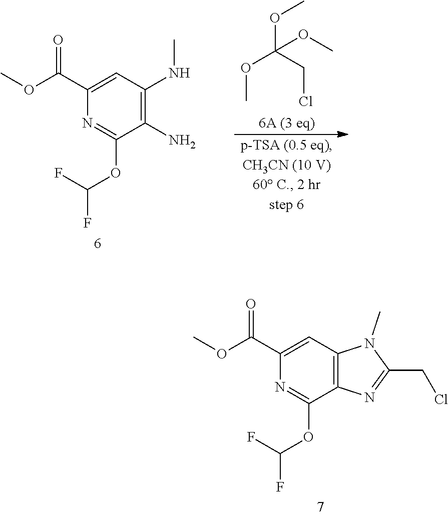

A mixture of 6 (110 mg, 444.99 μmol, 1 eq), 6A (206.37 mg, 1.33 mmol, 179.92 μL, 3 eq), p-TSA (38.31 mg, 222.49 μmol, 0.5 eq) in ACN (1 mL) was degassed and purged with N₂ for 3 times, and then the mixture was stirred at 60° C. for 2 hr under N₂ atmosphere. LCMS (product: RT=0.695 min) showed the 6 was consumed completely. The reaction mixture was concentrated under reduced pressure to give a residue. 7 (136.02 mg, crude) was obtained as a yellow solid.

LCMS: RT=0.659 min, MS cal.: 305.7, [M+H]⁺=306.1

### General Procedure for Preparation of Intermediate 8:

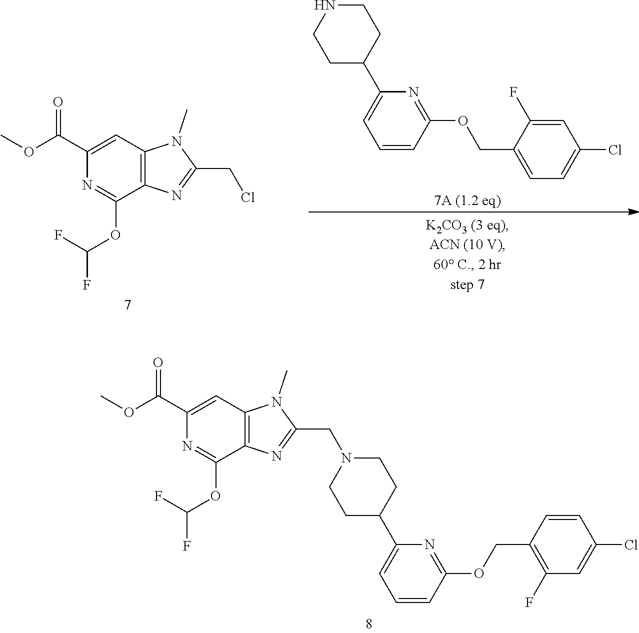 [SMILES: COC(=O)c1cc2c(nc(CCl)n2C)c(OC(F)F)n1 ; Fc1cc(Cl)ccc1COc1cccc(C2CCNCC2)n1 ; COC(=O)c1cc2c(nc(CN3CCC(c4cccc(OCc5ccc(Cl)cc5F)n4)CC3)n2C)c(OC(F)F)n1]

A mixture of 7 (136.02 mg, 445.00 μmol, 1 eq), 7A (171.30 mg, 534.00 μmol, 1.2 eq), K₂CO₃ (184.51 mg, 1.33 mmol, 3 eq) in ACN (1.5 mL) was degassed and purged with N₂ for 3 times, and then the mixture was stirred at 60° C. for 2 hr under N₂ atmosphere. LCMS (product: RT=0.959 min) showed the 7 was consumed completely. The reaction mixture was diluted with H₂O 10 mL and extracted with EtOAc (10 mL*3). The combined organic layers were washed with brine (10 mL*2), dried over Na₂SO₄, filtered and concentrated under reduced pressure to give a residue. The residue was purified by column chromatography (SiO₂, Petroleum ether/Ethyl acetate=100/1 to 1/1). 8 (190 mg, 322.04 μmol, 72.37% yield) was obtained as a white solid.

LCMS: RT=0.959 min, MS cal.: 590.0, [M+H]⁺=590.3

¹H NMR (400 MHz, DMSO-d₆) δ=8.33 (s, 1H), 7.91 (t, J=72.4 Hz, 1H), 7.62 (t, J=7.6 Hz, 1H), 7.55 (t, J=8.4 Hz, 1H), 7.45 (dd, J=2.0, 10.0 Hz, 1H), 7.28 (dd, J=1.6, 8.2 Hz, 1H), 6.87 (d, J=7.2 Hz, 1H), 6.67 (d, J=8.0 Hz, 1H), 5.36 (s, 2H), 4.02 (s, 3H), 3.91 (s, 3H), 3.89 (s, 2H), 2.94 (br d, J=11.2 Hz, 2H), 2.67-2.54 (m, 1H), 2.28-2.17 (m, 2H), 1.83-1.67 (m, 4H)

### General Procedure for Preparation of Compound 253 (9):

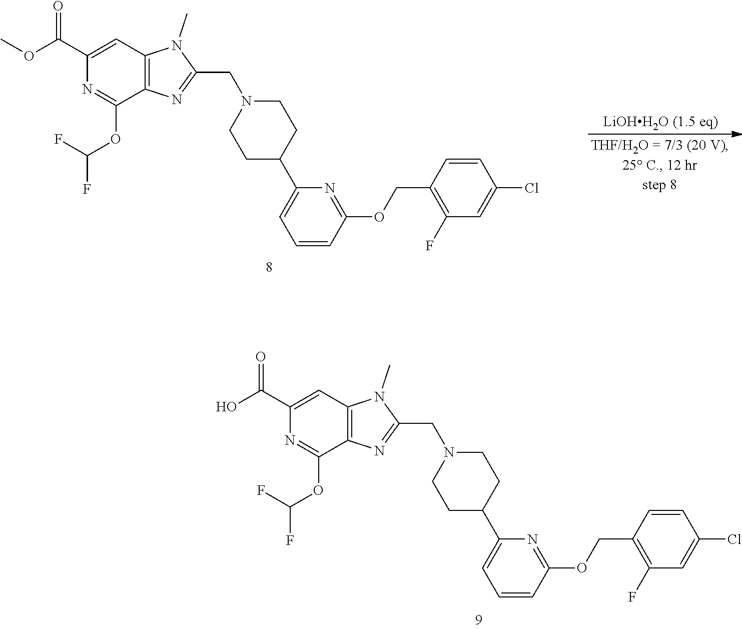 [SMILES: COC(=O)c1cc2c(nc(CN3CCC(c4cccc(OCc5ccc(Cl)cc5F)n4)CC3)n2C)c(OC(F)F)n1 ; Cn1c(CN2CCC(c3cccc(OCc4ccc(Cl)cc4F)n3)CC2)nc2c(OC(F)F)nc(C(=O)O)cc21]

To a solution of 7 (60 mg, 101.70 μmol, 1 eq) in THF (0.7 mL) and H₂O (0.3 mL) was added LiOH·H₂O (6.40 mg, 152.54 μmol, 1.5 eq). The mixture was stirred at 25° C. for 12 hr. LCMS (product: RT=1.684 min) showed the 7 was consumed completely. The crude product was purified by reversed-phase HPLC (column: Waters Xbridge Prep OBD C18 150*40 mm*10 um; mobile phase: [H₂O (10 mM NH₄HCO₃)-ACN]; gradient: 20%-70% B over 8.0 min). 2-((4-(6-((4-Chloro-2-fluorobenzyl)oxy)pyridin-2-yl)piperidin-1-yl)methyl)-4-(difluoromethoxy)-1-methyl-1H-imidazo[4,5-c]pyridine-6-carboxylic acid (24.54 mg, 42.61 μmol, 41.90% yield) was obtained as a white solid.

LCMS: RT=2.776 min, MS cal.: 576.0, [M+H]⁺=576.2

HPLC: RT=11.998 min, purity=99.847%

¹H NMR (400 MHz, MeOH-d4) δ=8.30 (s, 1H), 8.18 (br s, 1H), 8.12 (s, 1H), 7.94 (s, 1H), 7.58 (t, J=7.6 Hz, 1H), 7.48 (t, J=8.0 Hz, 1H), 7.23-7.13 (m, 2H), 6.83 (d, J=7.2 Hz, 1H), 6.63 (d, J=8.0 Hz, 1H), 5.40 (s, 2H), 4.06 (s, 2H), 4.03 (s, 3H), 3.17 (br d, J=11.2 Hz, 2H), 2.76-2.65 (m, 1H), 2.56-2.42 (m, 2H), 1.99-1.85 (m, 4H)

### Example A170—Preparation of Compound 168. 2-((4-(4-((4-Chloro-2-fluorobenzyl)oxy)pyrimidin-2-yl)piperidin-1-yl)methyl)-4-(difluoromethoxy)-1-methyl-1H-benzo[d]imidazole-6-carboxylic acid (Compound 168) (5)

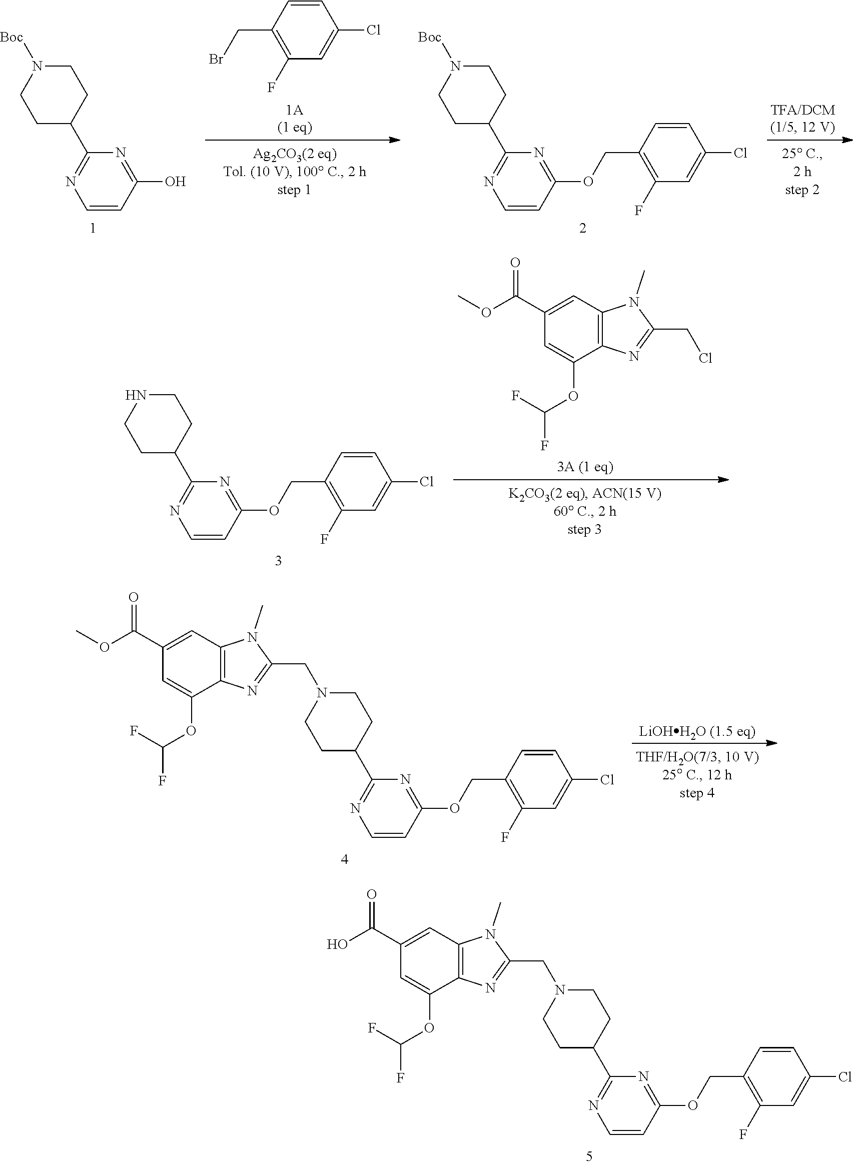

### General Procedure for Preparation of Intermediate 2:

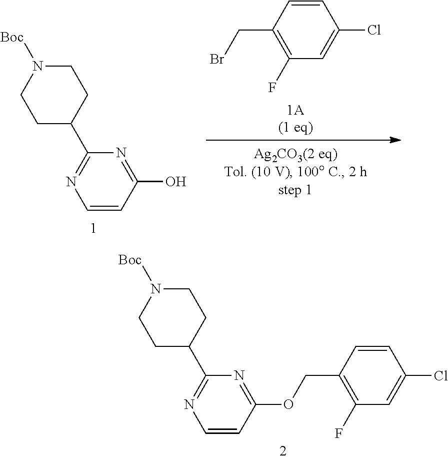 [SMILES: CC(C)(C)OC(=O)N1CCC(c2nccc(O)n2)CC1 ; Fc1cc(Cl)ccc1CBr ; CC(C)(C)OC(=O)N1CCC(c2nccc(OCc3ccc(Cl)cc3F)n2)CC1]

The reaction mixture was combined with the small scale (20 mg). Two reactions were combined for purification. To a solution of 1 (200 mg, 715.99 μmol, 1 eq) and 1A (192.00 mg, 859.19 μmol, 1.2 eq) in toluene (2 mL) was added Ag₂CO₃ (394.86 mg, 1.43 mmol, 64.98 μL, 2 eq) at 20° C. The mixture was stirred at 100° C. for 2 hr. LCMS (Rt=1.556) showed 1 was consumed completely and desired mass was detected. The reaction mixture was diluted with EtOAc (3 mL) and filtered through celite pad. The filter cake was rinsed with EtOAc (3 mL*2). The filtrate was diluted with H₂O (15 mL) and extracted with EtOAc (3 mL*2). The combined organic layers were washed with brine (5 mL*2), dried over Na₂SO₄, filtered, and concentrated under reduced pressure to give a crude product. The residue was purified by column chromatography (SiO₂, Petroleum ether/Ethyl acetate=10/1 to 1/1). 2 (300 mg, 711.08 μmol, 90% yield) was obtained as a white solid.

LCMS: RT=1.556 min, MS cal.: 421.2, [M+H]⁺=422.0

¹H NMR (400 MHz, CHCl3-d) δ=8.39 (d, J=5.6 Hz, 1H), 7.45-7.38 (t, J=8.4 Hz, 1H), 7.19-7.12 (m, 2H), 6.61 (d, J=5.6 Hz, 1H), 5.46 (s, 2H), 4.30-4.13 (s, 2H), 2.99-2.81 (m, 3H), 1.98 (d, J=12 Hz, 2H), 1.80 (dq, J=12.4, 4.4 Hz, 2H), 1.49 (s, 9H)

### General Procedure for Preparation of Intermediate 3

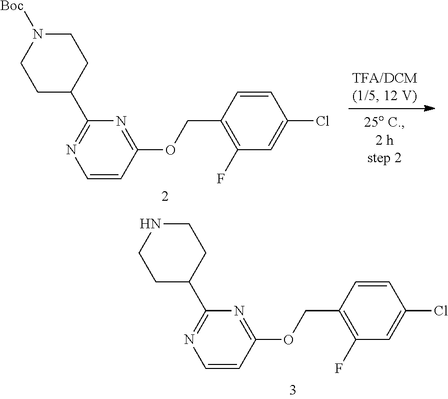 [SMILES: CC(C)(C)OC(=O)N1CCC(c2nccc(OCc3ccc(Cl)cc3F)n2)CC1 ; Fc1cc(Cl)ccc1COc1ccnc(C2CCNCC2)n1]

To a solution of 2 (250 mg, 592.57 μmol, 1 eq) in DCM (2.5 mL) was added TFA (0.5 mL) at 25° C. The mixture was stirred at 25° C. for 2 hr. TLC (Petroleum ether:Ethyl acetate=1:1, Rf=0.1) indicated 2 was consumed completely and one new spot formed. The reaction was clean according to TLC. The reaction mixture was added saturated sodium carbonate aqueous solution to adjust pH-8 at 0° C., diluted with H₂O (10 mL) and extracted with DCM (3 mL*3). The combined organic layers were washed with brine (5 mL*2), dried over Na₂SO₄, filtered, and concentrated under reduced pressure to give a crude product. Compound 3 (180 mg, 559.39 μmol, 94% yield) was obtained as a white solid.

¹H NMR (400 MHz, DMSO-d6) δ=8.47 (d, J=5.6 Hz, 1H), 7.60 (t, J=8.0 Hz, 1H), 7.50 (dd, J=10.0, 2.0 Hz, 1H), 7.33 (dd, J=8.4, 1.6 Hz, 1H), 6.81 (d, J=6.0 Hz, 1H), 5.45 (s, 2H), 3.10 (d, J=12.0 Hz, 2H), 2.86 (tt, J=11.2, 3.6 Hz, 1H), 2.71 (td, J=12.4, 2.4 Hz, 2H), 1.96-1.85 (dd, J=12.8, 2.0 Hz, 2H), 1.78-1.64 (m, 2H)

### General Procedure for Preparation of Intermediate 4:

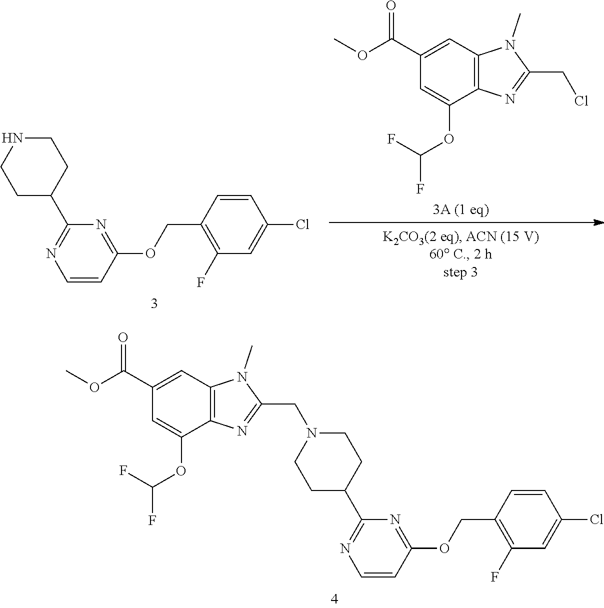 [SMILES: Fc1cc(Cl)ccc1COc1ccnc(C2CCNCC2)n1 ; COC(=O)c1cc(OC(F)F)c2nc(CCl)n(C)c2c1 ; COC(=O)c1cc(OC(F)F)c2nc(CN3CCC(c4nccc(OCc5ccc(Cl)cc5F)n4)CC3)n(C)c2c1]

To a solution of 3 (130 mg, 404.01 μmol, 1 eq) and 3A (123.09 mg, 404.01 μmol, 1 eq) in ACN (2 mL) was added K₂CO₃ (111.67 mg, 808.01 μmol, 2 eq) at 20° C. The mixture was stirred at 60° C. for 2 hr. LCMS (Rt=1.531) showed 3 was consumed completely and desired mass was detected. The reaction mixture was added H₂O (10 mL), and then extracted with EtOAc (5 mL*3). The combined organic layers were washed with brine (10 mL*2), dried over Na₂SO₄, filtered, and concentrated under reduced pressure to give a crude product. 4 (260 mg, crude) was obtained as a white solid.

LCMS: RT=1.531 min, MS cal.: 589.2, [M+H]⁺=590.0

### General Procedure for Preparation of Compound 168 (5):

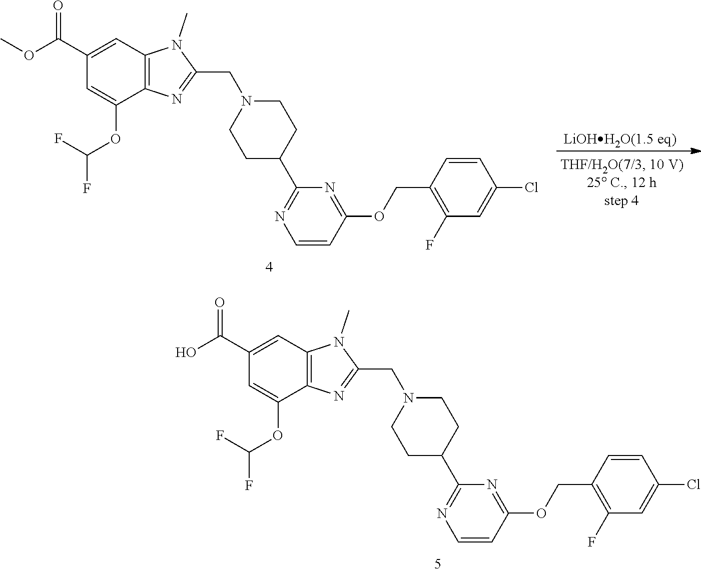 [SMILES: COC(=O)c1cc(OC(F)F)c2nc(CN3CCC(c4nccc(OCc5ccc(Cl)cc5F)n4)CC3)n(C)c2c1 ; Cn1c(CN2CCC(c3nccc(OCc4ccc(Cl)cc4F)n3)CC2)nc2c(OC(F)F)cc(C(=O)O)cc21]

To a solution of 4 (100 mg, 169.49 μmol, 1 eq) in THF (0.7 mL) was added LiOH·H₂O (10.67 mg, 254.24 μmol, 1.5 eq) in H₂O (0.3 mL) at 25° C. The mixture was stirred at 25° C. for 12 hr.

LCMS (Rt=1.544) showed 4 was consumed completely and desired mass was detected. The reaction mixture was filtered and concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 15%-55% B over 8.0 min). 2-((4-(4-((4-Chloro-2-fluorobenzyl)oxy)pyrimidin-2-yl)piperidin-1-yl)methyl)-4-(difluoromethoxy)-1-methyl-1H-benzo[d]imidazole-6-carboxylic acid (29.17 mg, 50.65 μmol, 30% yield) was obtained as a white solid.

LCMS: RT=1.544 min, MS cal.: 575.2, [M+H]⁺=576.1

HPLC: RT=10.626 purity: 97.25%

¹H NMR (400 MHz, MeOH-d4) δ=8.46 (d, J=5.2 Hz, 1H), 8.07 (d, 1H), 7.63 (t, J=74.4 Hz, 1H), 7.58 (t, J=8.4 Hz, 1H), 7.52 (s, 1H), 7.48 (dd, J=10.4, 1.6 Hz, 1H), 7.31 (dd, J=8.0, 1.6 Hz, 1H), 6.80 (d, J=5.6 Hz, 1H), 5.44 (s, 2H), 3.96 (s, 3H), 3.85 (s, 2H), 2.91 (d, J=11.2 Hz, 2H), 2.78-2.68 (tt, J=3.6, 11.4 Hz, 1H), 2.24 (t, J=11.2 Hz, 2H), 1.95-1.85 (d, J=10.8 Hz, 2H), 1.84-1.70 (m, 2H)

### Example A171—Preparation of Compound 169. 2-((4-(6-((4-Chloro-2-fluorobenzyl)oxy)pyridin-2-yl)piperidin-1-yl)methyl)-4-(2,2-difluoroethoxy)-1-methyl-1H-benzo[d]imidazole-6-carboxylic acid (Compound 169) (8)

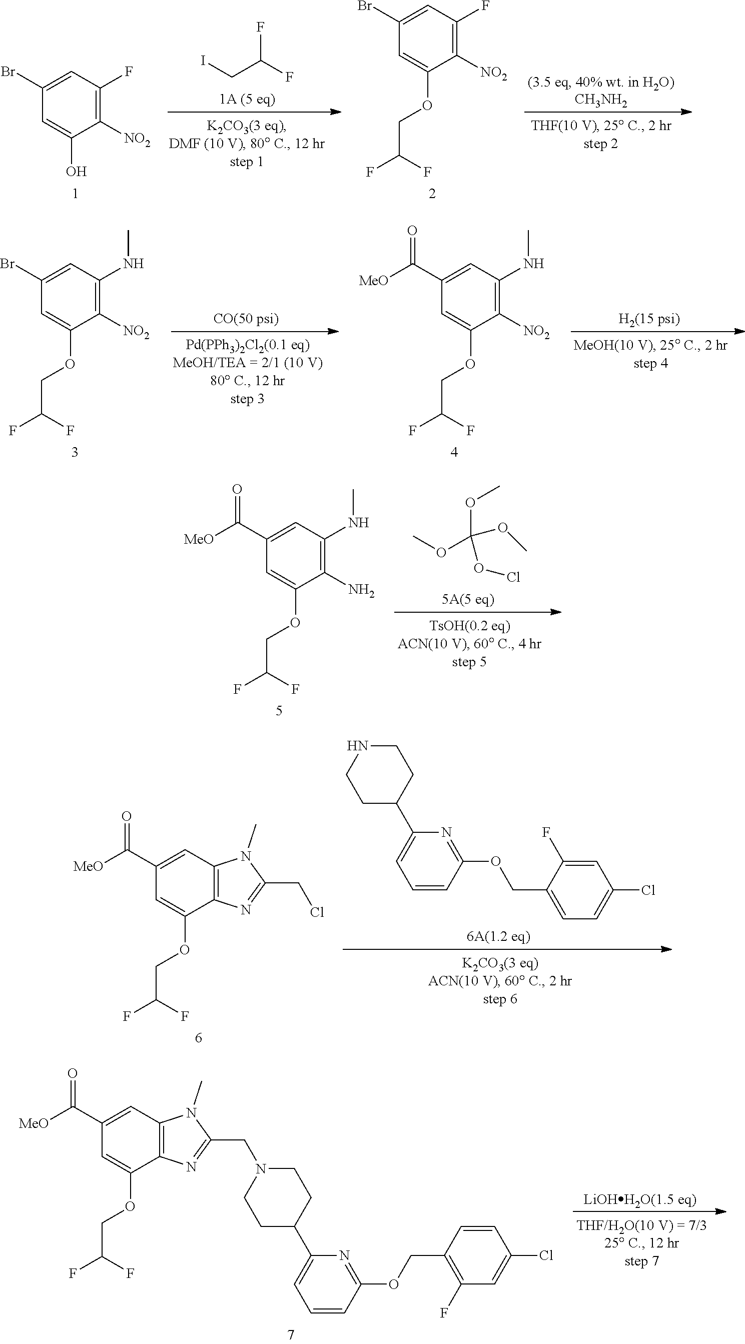

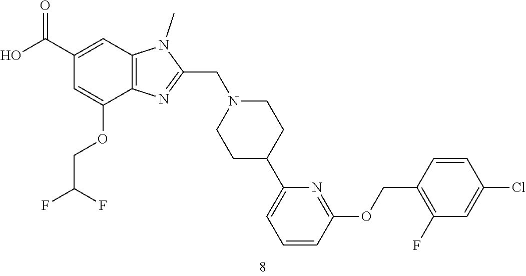 [SMILES: Cn1c(CN2CCC(c3cccc(OCc4ccc(Cl)cc4F)n3)CC2)nc2c(OCC(F)F)cc(C(=O)O)cc21]

### General Procedure for Preparation of Intermediate 2:

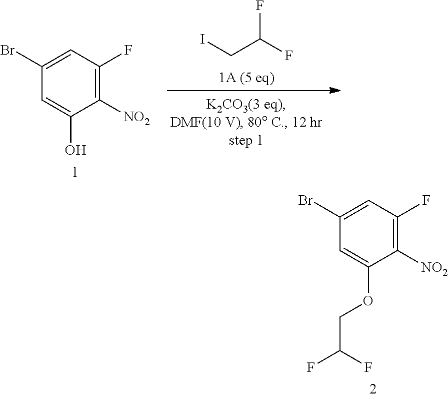 [SMILES: O=[N+]([O-])c1c(O)cc(Br)cc1F ; FC(F)CI ; O=[N+]([O-])c1c(F)cc(Br)cc1OCC(F)F]

To a solution of 1 (1 g, 4.24 mmol, 1 eq) in DMF (10 mL) was added K₂CO₃ (1.76 g, 12.71 mmol, 3 eq) and 1A (4.07 g, 21.19 mmol, 5 eq) at 25° C. The mixture was stirred at 80° C. for 12 hr. TLC (PE:EtOAc=3:1) indicated 1 was consumed completely and one new spot formed. The reaction was clean according to TLC. The mixture was diluted with H₂O (30 mL), and then extracted with EtOAc (20 mL*2). The combined organic layers were washed with brine (20 mL*2), dried over Na₂SO₄, filtered and concentrated under reduced pressure to give a residue. The residue was purified by column chromatography (SiO₂, PE/EtOAc=10/1 to 0/1). 2 (1.2 g, 4.00 mmol, 94.39% yield) was obtained as a yellow solid.

¹H NMR (400 MHz, CHCl3-d) δ=7.18-7.11 (m, 1H), 7.04 (t, J=2.0 Hz, 1H), 6.09 (tt, J=54.4, 4.0 Hz 1H), 4.31 (td, J=12.4, 4.0 Hz, 2H)

### General Procedure for Preparation of Intermediate 3:

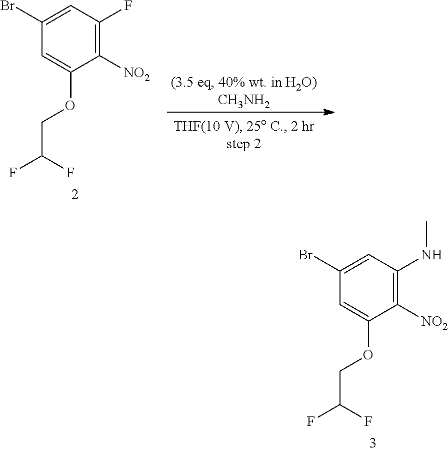 [SMILES: O=[N+]([O-])c1c(F)cc(Br)cc1OCC(F)F ; CNc1cc(Br)cc(OCC(F)F)c1[N+](=O)[O-]]

Equip a 100 mL three-necked round bottom flask, thermometer, N₂ balloon. THF (12 mL) was charged to a three-necked round bottom flask, then 2 (1.2 g, 4.00 mmol, 1 eq) was added to the mixture at 25° C. CH₃NH₂ (1.09 g, 14.00 mmol, 40% purity, 3.5 eq) was added dropwise to the reaction mixture at 25° C. After the addition, the mixture was stirred at 25° C. for 2 hr. TLC (PE:EtOAc=3:1) indicated 2 was consumed completely and one new spot formed. The reaction was clean according to TLC. LC-MS (Rt=0.791 min) showed 2 was consumed completely and one main peak with desired mass was detected. The reaction mixture was diluted with H₂O (30 mL), added FA (1 M) to pH=5 at 25° C., and then extracted with EtOAc (20 mL*2). The combined organic layers were washed with brine (20 mL*2), dried over Na₂SO₄, filtered and concentrated under reduced pressure to give 3 (0.97 g, 3.12 mmol, 77.96% yield) was obtained as a yellow solid.

LCMS: Rt=0.791 min, MS cal.: 309.98/310.98, [M+H]⁺=309.10/311.10

¹H NMR (400 MHz, CHCl3-d) δ=6.64 (d, J=2.0 Hz, 1H), 6.55-6.47 (m, 1H), 6.40 (d, J=2.0 Hz, 1H), 6.12 (tt, J=54.8, 4.4 Hz 1H), 4.24 (td, J=12.4, 4.0 Hz, 2H), 2.93 (s, 3H)

### General Procedure for Preparation of Intermediate 4:

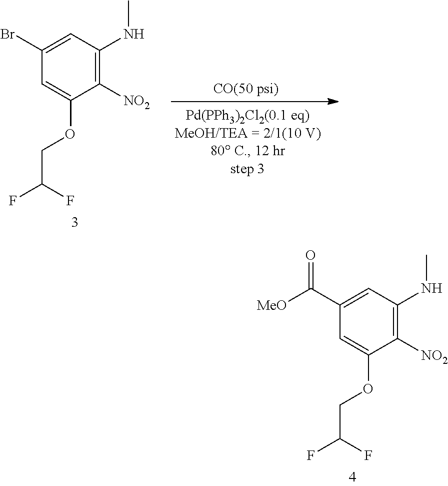 [SMILES: CNc1cc(Br)cc(OCC(F)F)c1[N+](=O)[O-] ; CNc1cc(C(=O)OC)cc(OCC(F)F)c1[N+](=O)[O-]]

3 (1.58 g, 5.08 mmol, 1 eq) was charged to the round bottom flask, then starting TEA (8 mL) and MeOH (16 mL), Pd(PPh₃)₂Cl₂ (356.50 mg, 507.91 μmol, 0.1 eq) was added to the mixture at 25° C. After the addition, the mixture was stirred at 80° C. for 12 hr under CO (50 Psi). LC-MS (Rt=1.154 min) showed 3 was consumed completely and one main peak with desired mass was detected. The reaction mixture was filtered. The filtrate was diluted with H₂O (50 mL), the aqueous layer was extracted with EtOAc (50 mL*3). The combined organic phase was washed with brine (50 mL*2), dried with anhydrous Na₂SO₄, filtered and concentrated in vacuum. The residue was purified by column chromatography (SiO₂, PE/EtOAc=10/1 to 0/1). 4 (0.97 g, 3.34 mmol, 65.81% yield) was obtained as orange solid.

LCMS: Rt=1.154 min, MS cal.: 290.07/291.07, [M+H]⁺=290.9/291.9

¹H NMR (400 MHz, CHCl3-d) δ=7.16 (d, J=1.2 Hz, 1H), 6.92-6.89 (m, 1H), 6.12 (tt, J=54.8, 4.4 Hz, 2H), 4.31 (td, J=12.4, 4.0 Hz, 2H), 3.96 (s, 3H), 2.99 (s, 3H).

### General Procedure for Preparation of Intermediate 5:

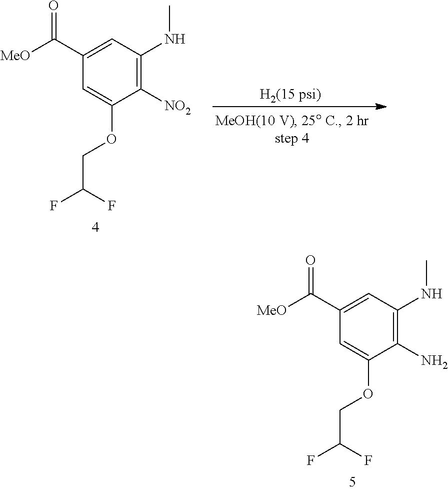 [SMILES: CNc1cc(C(=O)OC)cc(OCC(F)F)c1[N+](=O)[O-] ; CNc1cc(C(=O)OC)cc(OCC(F)F)c1N]

Equip a 100 mL single round bottom flask, addition funnel and thermometer, H₂ balloon. MeOH (11 mL) was charged to the single round bottom flask, then Pd/C (0.55 g) was added to the mixture at 25° C. under H₂ (15 psi). 4 (1.1 g, 3.79 mmol, 1 eq) was added in portions to the reaction mixture at 25° C. under H₂ (15 Psi). After the addition, the mixture was stirred at 25° C. for 2 hr under H₂ (15 Psi). LC-MS (Rt=0.993 min) showed 4 was consumed completely and one main peak with desired mass was detected. TLC (PE:EtOAc=3:1) indicated 4 was consumed completely and one new spot formed. The reaction was clean according to TLC. The suspension was filtered, and the filter cake was washed with MeOH (50 mL*3). The combined filtrates were concentrated to dryness to give 5 (1.2 g, crude) was obtained as gray solid.

LCMS: Rt=0.983 min, MS cal.: 260.10/261.10, [M+H]⁺=260.90/261.90

¹H NMR (400 MHz, CHCl3-d) δ=7.16 (dd, J=6.0, 1.2 Hz, 2H), 6.11 (tt, J=54.8, 4.0 Hz, 2H), 4.28 (td, J=13.2, 4.0 Hz, 2H), 3.89 (s, 3H), 2.93 (s, 3H)

### General Procedure for Preparation of Intermediate 6:

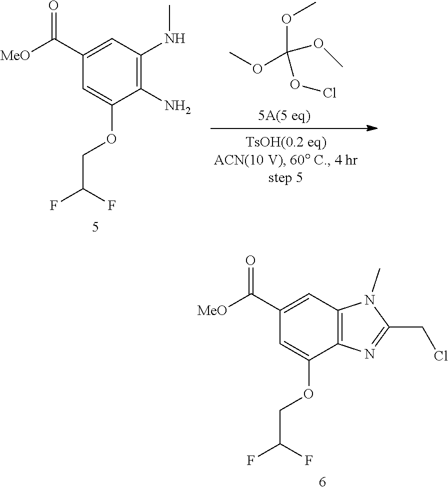

To a solution of 5 (200 mg, 768.53 μmol, 1 eq) in ACN (2 mL) was added TsOH (26.47 mg, 153.71 μmol, 0.2 eq) and 5A (356.43 mg, 2.31 mmol, 310.75 μL, 3 eq) at 25° C. The mixture was stirred at 60° C. for 2 hr. After monitoring, the reaction was not complete. Then was added 5A (237.62 mg, 1.54 mmol, 207.16 μL, 2 eq) at 25° C. The mixture was stirred at 60° C. for 2 hr. LC-MS (Rt=0.430 min) showed 5 was consumed completely and one main peak with desired mass was detected. The mixture was diluted with H₂O (10 mL), extracted with EtOAc (10 mL*3). The combined organic layer was washed with 10 mL H₂O, 10 mL brine, dried over Na₂SO₄, filtered and the filtrate was concentrated to give a residue. The residue was purified by column chromatography (SiO₂, PE/EtOAc=10/1 to 0/1). 6 (140 mg, 439.28 μmol, 57.16% yield) was obtained as a green solid.

LCMS: Rt=0.430 min, MS cal.: 318.06/320.06, [M+H]⁺=319.0/321.0

¹H NMR (400 MHz, CHCl3-d) δ=7.85 (d, J=1.2 Hz, 1H), 7.45 (s, 1H), 6.29 (tt, J=55.2, 4.0 Hz, 1H), 4.89 (s, 2H), 4.55 (td, J=13.2, 4.4 Hz, 2H), 3.98 (s, 3H), 3.94 (s, 3H), 0.08 (s,

### General Procedure for Preparation of Intermediate 7:

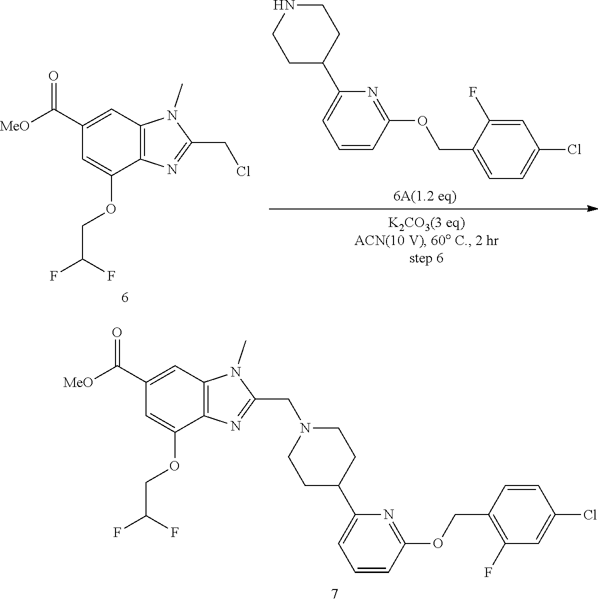 [SMILES: COC(=O)c1cc(OCC(F)F)c2nc(CCl)n(C)c2c1 ; Fc1cc(Cl)ccc1COc1cccc(C2CCNCC2)n1 ; COC(=O)c1cc(OCC(F)F)c2nc(CN3CCC(c4cccc(OCc5ccc(Cl)cc5F)n4)CC3)n(C)c2c1]

To a solution of 6 (140 mg, 439.28 μmol, 1 eq) in ACN (2 mL) was added K₂CO₃ (182.13 mg, 1.32 mmol, 3 eq) and 6A (169.10 mg, 527.14 μmol, 1.2 eq). The mixture was stirred at 60° C. for 2 hr. LC-MS (Rt=0.509 min) showed 6 was consumed completely and one main peak with desired mass was detected. The mixture was diluted with H₂O (30 mL), extracted with EtOAc (10 mL*3). The combined organic layer was washed with 20 mL H₂O, 20 mL brine, dried over Na₂SO₄, filtered and the filtrate was concentrated to give a crude product. The residue was purified by column chromatography (SiO₂, PE/EtOAc=10/1 to 0/1). 7 (190 mg, 315.08 μmol, 71.73% yield) was obtained as a green solid.

LCMS: Rt=0.509 min, MS cal.: 602.19/603.19, [M+H]⁺=603.20/604.10

¹H NMR (400 MHz, CHCl3-d) δ=7.84 (s, 1H), 7.53-7.40 (m, 3H), 7.14-7.08 (m, 2H), 6.74 (d, J=7.2 Hz, 1H), 6.61 (d, J=8.4 Hz, 1H), 6.29 (tt, J=55.6, 4.0 Hz, 1H), 5.41 (s, 2H), 4.56 (td, J=13.2, 4.4 Hz, 2H), 4.00 (br s, 3H), 3.97 (s, 3H), 3.95-3.87 (m, 2H), 3.05-2.88 (m, 2H), 2.69-2.55 (m, 1H), 2.30 (br s, 2H), 1.96-1.80 (m, 4H)

### General Procedure for Preparation of Compound 169 (8):

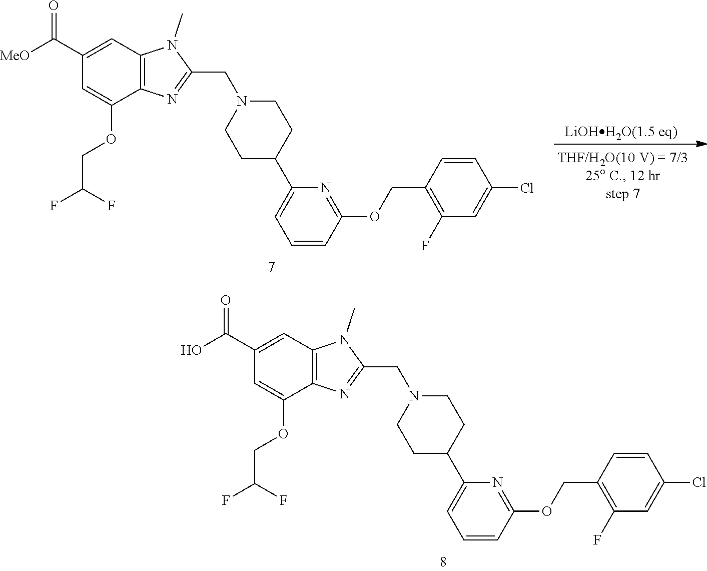 [SMILES: COC(=O)c1cc(OCC(F)F)c2nc(CN3CCC(c4cccc(OCc5ccc(Cl)cc5F)n4)CC3)n(C)c2c1 ; Cn1c(CN2CCC(c3cccc(OCc4ccc(Cl)cc4F)n3)CC2)nc2c(OCC(F)F)cc(C(=O)O)cc21]

To a solution of 7 (100 mg, 165.83 μmol, 1 eq) in THF (0.7 mL) was added LiOH·H₂O (10.44 mg, 248.74 μmol, 1.5 eq) in H₂O (0.3 mL). The mixture was stirred at 25° C. for 12 hr. LC-MS (Rt=0.451 min) showed 7 was consumed completely and one main peak with desired mass was detected. The reaction was poured into H₂O and ACN, and adjusted to pH=7 with 0.5 M CA. The residue was purified by prep-HPLC (column: Waters Xbridge Prep OBD C18 150*40 mm*10 um; mobile phase: [H₂O (10 mM NH₄HCO₃)-ACN]; gradient: 5%-55% B over 8.0 min). 2-((4-(6-((4-Chloro-2-fluorobenzyl)oxy)pyridin-2-yl)piperidin-1-yl)methyl)-4-(2,2-difluoroethoxy)-1-methyl-1H-benzo[d]imidazole-6-carboxylic acid (18.39 mg, 31.22 μmol, 18.83% yield) was obtained as a yellow solid.

LCMS: Rt=2.802 min, MS cal.: 588.18/589.18, [M+H]⁺=589.20/590.20

HPLC: Rt=11.895 min, purity: 93.88%

¹H NMR (400 MHz, DMSO-d₆) δ=7.85 (s, 1H), 7.65 (s, 1H), 7.57-7.52 (m, 1H), 7.47-7.42 (m, 1H), 7.32-7.30 (m, 1H), 7.29-7.25 (m, 1H), 6.88-6.84 (m, 1H), 6.68-6.64 (m, 1H), 6.47 (tt, J=54.8, 3.2 Hz, 1H), 5.35 (s, 2H), 4.62 (td, J=14.4, 3.2 Hz, 2H), 3.93-3.91 (m, 3H), 3.83-3.81 (m, 2H), 2.96-2.89 (m, 2H), 2.58 (br s, 1H), 2.24-2.16 (m, 2H), 1.82-1.65 (m, 4H)

### Example A172—Preparation of Compound 170. 2-((4-(6-((4-Chloro-2-fluorobenzyl)oxy)pyridin-2-yl)piperidin-1-yl)methyl)-4-(2-fluoroethoxy)-1-methyl-1H-benzo[d]imidazole-6-carboxylic acid (Compound 170) (3)

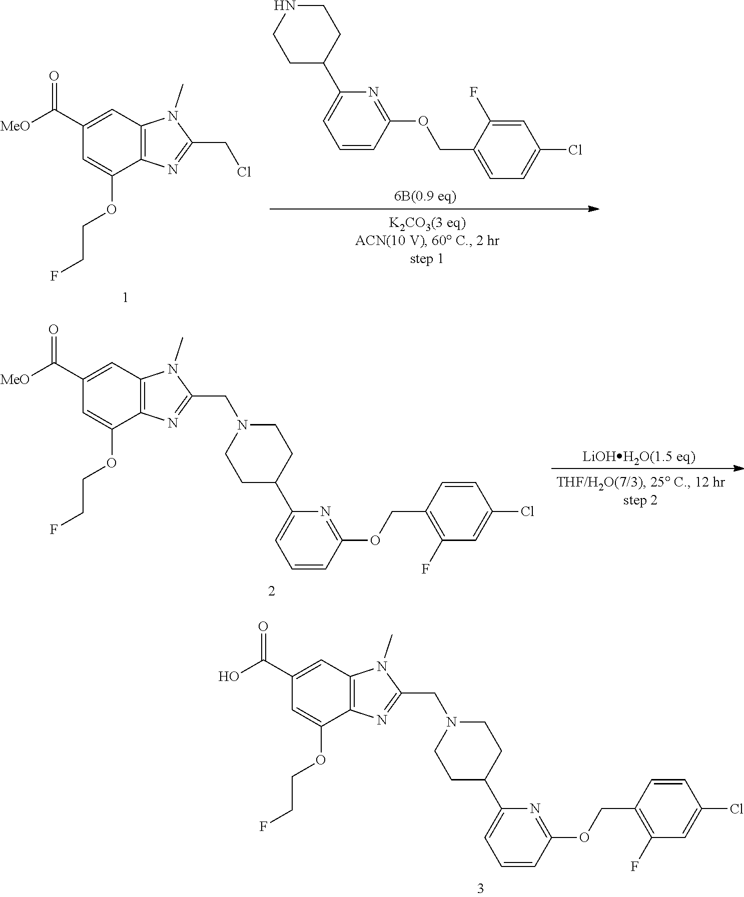 [SMILES: COC(=O)c1cc(OCCF)c2nc(CCl)n(C)c2c1 ; Fc1cc(Cl)ccc1COc1cccc(C2CCNCC2)n1 ; COC(=O)c1cc(OCCF)c2nc(CN3CCC(c4cccc(OCc5ccc(Cl)cc5F)n4)CC3)n(C)c2c1 ; Cn1c(CN2CCC(c3cccc(OCc4ccc(Cl)cc4F)n3)CC2)nc2c(OCCF)cc(C(=O)O)cc21]

### General Procedure for Preparation of Intermediate 2:

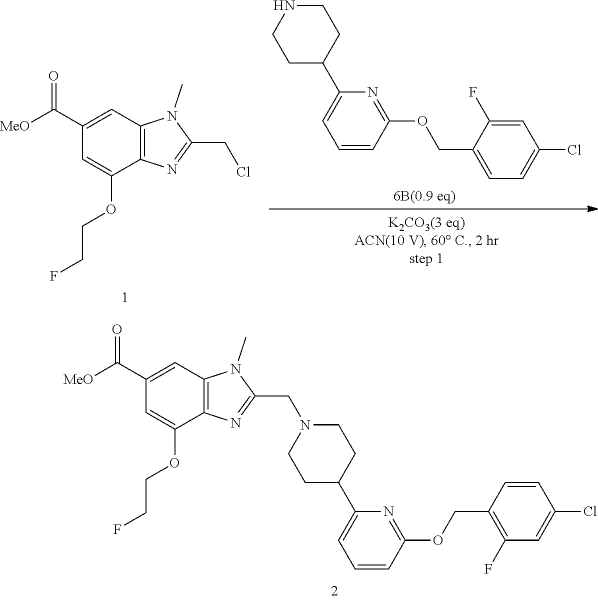 [SMILES: COC(=O)c1cc(OCCF)c2nc(CCl)n(C)c2c1 ; Fc1cc(Cl)ccc1COc1cccc(C2CCNCC2)n1 ; COC(=O)c1cc(OCCF)c2nc(CN3CCC(c4cccc(OCc5ccc(Cl)cc5F)n4)CC3)n(C)c2c1]

To a solution of 1 (100 mg, 332.54 μmol, 1 eq) in ACN (1 mL) was added K₂CO₃ (137.88 mg, 997.63 μmol, 3 eq) and 6B (128.01 mg, 399.05 μmol, 1.2 eq.) at 25° C. The mixture was stirred at 60° C. for 2 hr. TLC showed 1 was consumed completely. The reaction mixture was diluted with H₂O 10 mL and extracted with EtOAc 9 mL. The combined organic layers were washed with brine 10 mL, dried over N₂SO₄, filtered and concentrated under reduced pressure to give 2 (150 mg, 256.39 μmol, 77.10% yield) as a yellow solid.

LCMS: RT=1.617 MS cal.: 584.20, [M−H]⁻=585.0

### General Procedure for Preparation of Compound 170 (3)

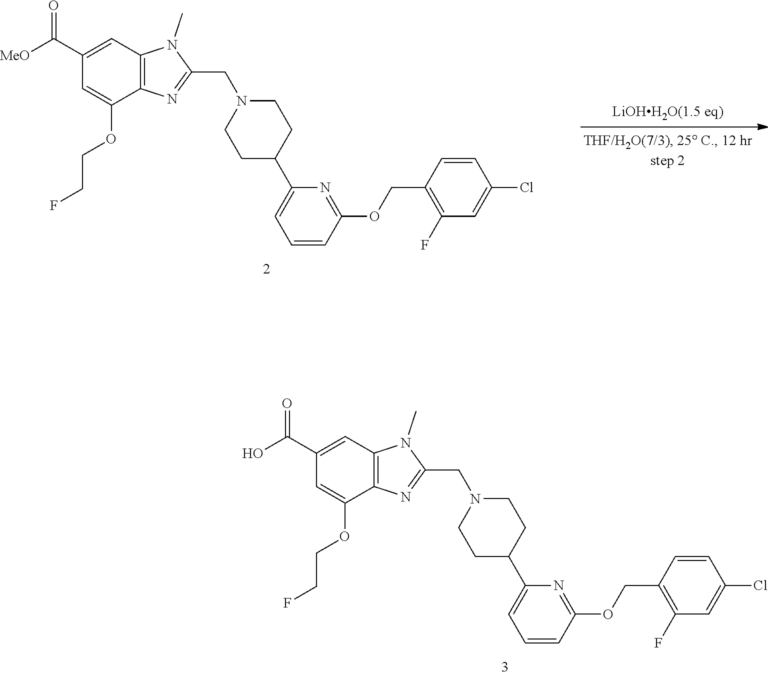 [SMILES: COC(=O)c1cc(OCCF)c2nc(CN3CCC(c4cccc(OCc5ccc(Cl)cc5F)n4)CC3)n(C)c2c1 ; Cn1c(CN2CCC(c3cccc(OCc4ccc(Cl)cc4F)n3)CC2)nc2c(OCCF)cc(C(=O)O)cc21]

To a solution of 2 (150 mg, 256.39 μmol, 1 eq) in THF (0.7 mL) was added LiOH·H₂O (16.14 mg, 384.59 μmol, 1.5 eq) and H₂O (0.3 mL) at 25° C. The mixture was stirred at 25° C. for 12 hr. TLC showed 2 was consumed completely. The reaction mixture was added with 0.5 M citric acid to adjust pH=7. The residue was purified by prep-HPLC (column: Waters Xbridge Prep OBD C18 150*40 mm*10 um; mobile phase: [H₂O (10 mM NH₄HCO₃)-ACN]; gradient: 5%-55% B over 8.0 min). 2-((4-(6-((4-Chloro-2-fluorobenzyl)oxy)pyridin-2-yl)piperidin-1-yl)methyl)-4-(2-fluoroethoxy)-1-methyl-1H-benzo[d]imidazole-6-carboxylic acid (29.33 mg, 51.36 μmol, 20.03% yield) was obtained as a white solid.

LCMS: RT=2.790 min, MS cal.: 570.18, [M+H]⁻=571.2

¹H NMR (400 MHz, CHCl3-d) δ=7.81 (s, 1H), 7.62 (t, J=7.6 Hz, 1H), 7.55 (t, J=8.4 Hz, 1H), 7.45 (dd, J=2.4, 10.4 Hz, 1H), 7.30-7.26 (m, 2H), 6.86 (d, J=7.2 Hz, 1H), 6.66 (d, J=8.0 Hz, 1H), 5.35 (s, 2H), 4.89 (dt, J=48.0 Hz, 3.6 Hz, 2H), 4.54 (dt, J=30.4 Hz, 3.6 Hz, 2H), 3.92 (s, 3H), 3.82 (s, 2H), 2.92 (d, J=11.2 Hz, 2H), 2.61-2.56 (m, 1H), 2.19 (t, J=10.4 Hz, 2H), 1.82-1.75 (m, 2H), 1.74-1.64 (m, 2H)

### Example A173—Preparation of Compound 171. 2-((4-(3-((4-Chloro-2-fluorobenzyl)oxy)-2-methoxyphenyl)piperidin-1-yl)methyl)-4-(difluoromethoxy)-1-methyl-1H-benzo[d]imidazole-6-carboxylic acid (Compound 171) (5)

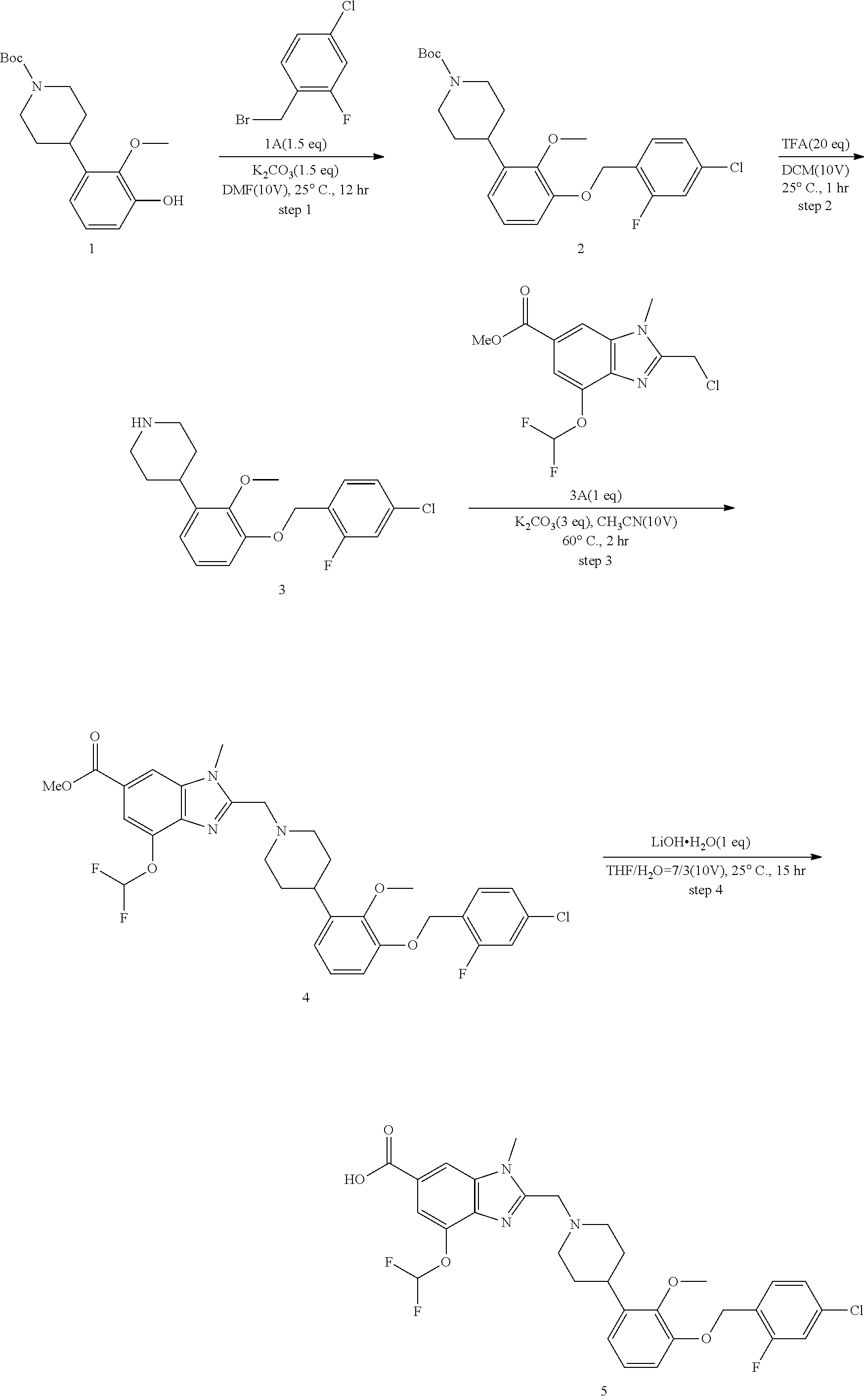

### General Procedure for Preparation of Intermediate 2:

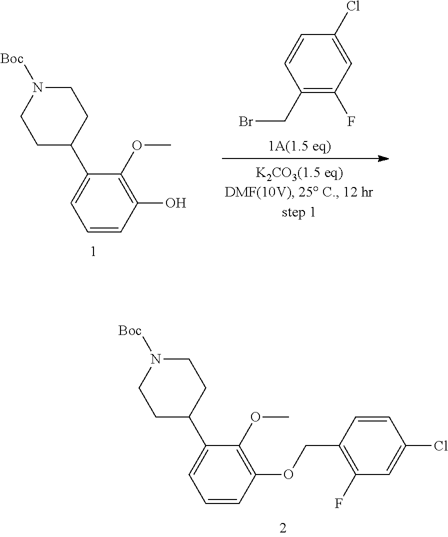 [SMILES: COc1c(O)cccc1C1CCN(C(=O)OC(C)(C)C)CC1 ; Fc1cc(Cl)ccc1CBr ; COc1c(OCc2ccc(Cl)cc2F)cccc1C1CCN(C(=O)OC(C)(C)C)CC1]

To a solution of 1 (400 mg, 1.30 mmol, 1 eq) in DMF (1 mL) was added K₂CO₃ (269.77 mg, 1.95 mmol, 1.5 eq) and 1A (290.80 mg, 1.30 mmol, 1 eq). The mixture was stirred at 25° C. for 2 hr. TLC (SiO₂, Petroleum ether:Ethyl acetate=1:1, Rf=0.70) showed the reaction was completed. The residue was poured into water (50 mL). The aqueous phase was extracted with ethyl acetate (20 mL*3). The combined organic phase was washed with saturated brine (50 mL*1), dried with anhydrous Na₂SO₄, filtered, and concentrated in vacuum. The residue was purified by column chromatography (SiO₂, PE/EtOAc=1/0 to 0/1). 2 (580 mg, 1.23 mmol, 94.42% yield) was obtained as a yellow solid.

HPLC: product: RT=4.033 min, purity: 95.32%

¹H NMR (400 MHz, MeOH-d4) δ=7.55 (t, J=8.0 Hz, 1H), 7.26 (br d, J=2.0 Hz, 1H), 7.24 (s, 1H), 7.04-6.99 (m, 1H), 6.96-6.92 (m, 1H), 6.83 (d, J=7.7 Hz, 1H), 5.13 (s, 2H), 4.20 (br d, J=12.8 Hz, 2H), 3.80 (s, 3H), 3.14 (br t, J=12.0 Hz, 1H), 2.87 (br s, 2H), 1.73 (br d, J=12.4 Hz, 2H), 1.63-1.58 (m, 1H), 1.57-1.52 (m, 1H), 1.48 (s, 9H)

### General Procedure for Preparation of Intermediate 3:

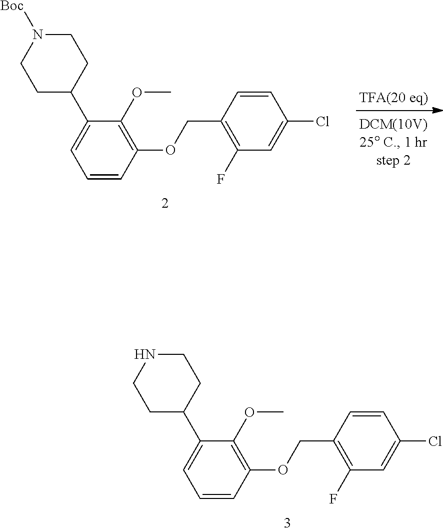 [SMILES: COc1c(OCc2ccc(Cl)cc2F)cccc1C1CCN(C(=O)OC(C)(C)C)CC1 ; COc1c(OCc2ccc(Cl)cc2F)cccc1C1CCNCC1]

To a solution of 2 (300 mg, 666.75 μmol, 1 eq) in DCM (2 mL) was added TFA (736.80 mg, 6.46 mmol, 480.00 μL, 9.69 eq). The mixture was stirred at 25° C. for 1 h. LC-MS showed 2 was consumed completely and one main peak with desired mass was detected. The mixture was filtered and concentrated in vacuum. The crude product was used into the next step without further purification. 3 (220 mg, 608.32 μmol, 91.24% yield) was obtained as a yellow solid.

LCMS: RT=0.414 min, MS cal.: 349.1, [M+H]⁺=350.0

HPLC: product: RT=3.332 min, purity: 96.73%

¹H NMR (400 MHz, MeOH-d4) δ=7.55 (t, J=8.0 Hz, 1H), 7.28-7.23 (m, 2H), 7.06-7.01 (m, 1H), 6.98-6.94 (m, 1H), 6.88 (dd, J=1.2, 7.6 Hz, 1H), 5.13 (s, 2H), 3.81 (s, 3H), 3.26 (br d, J=12.4 Hz, 2H), 3.17 (tt, J=3.6, 11.6 Hz, 1H), 2.89 (dt, J=3.2, 12.4 Hz, 2H), 1.87-1.69 (m, 4H)

### General Procedure for Preparation of Intermediate 4:

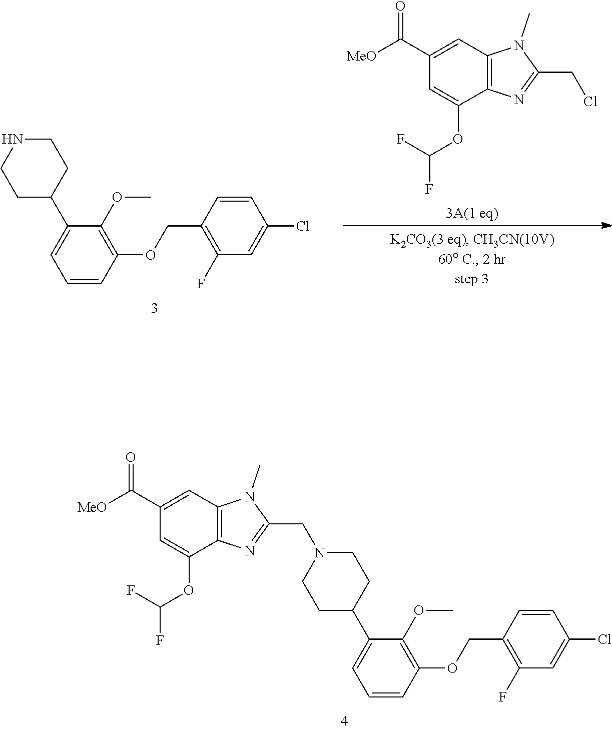 [SMILES: COc1c(OCc2ccc(Cl)cc2F)cccc1C1CCNCC1 ; COC(=O)c1cc(OC(F)F)c2nc(CCl)n(C)c2c1 ; COC(=O)c1cc(OC(F)F)c2nc(CN3CCC(c4cccc(OCc5ccc(Cl)cc5F)c4OC)CC3)n(C)c2c1]

To a solution of 3A (70 mg, 229.75 μmol, 1 eq) in ACN (1 mL) was added K₂CO₃ (95.26 mg, 689.26 μmol, 3 eq) and 3 (104.49 mg, 298.68 μmol, 1.3 eq). The mixture was de-gassed under reduced pressure and recharged with N₂. The mixture was stirred at 60° C. for 1 hr. LC-MS showed 3 was consumed completely and one main peak with desired mass was detected. The mixture was filtered and concentrated in vacuum. The residue was purified by prep-TLC (SiO₂, Petroleum ether:Ethyl acetate=2:1, Rf=0.40). 4 (121 mg, 191.39 μmol, 83.30% yield) was obtained as a yellow solid.

LCMS: RT=0.983 min, MS cal.: 617.2, [M+H]⁺=618.3

HPLC: product: RT=3.462 min, purity: 97.76%

¹H NMR (400 MHz, CHCl3-d) δ=7.99 (s, 1H), 7.72 (s, 1H), 7.52-7.48 (m, 1H), 7.33 (t, J=72.0 Hz, 1H), 7.18-7.11 (m, 2H), 7.04-6.98 (m, 1H), 6.85 (dd, J=8.0, 10.0 Hz, 2H), 5.12 (s, 2H), 4.00 (s, 3H), 3.97 (s, 3H), 3.90 (s, 2H), 3.85 (s, 3H), 3.06-2.92 (m, 3H), 2.35 (s, 2H), 1.84-1.67 (m, 4H)

### General Procedure for Preparation of Compound 171 (5):

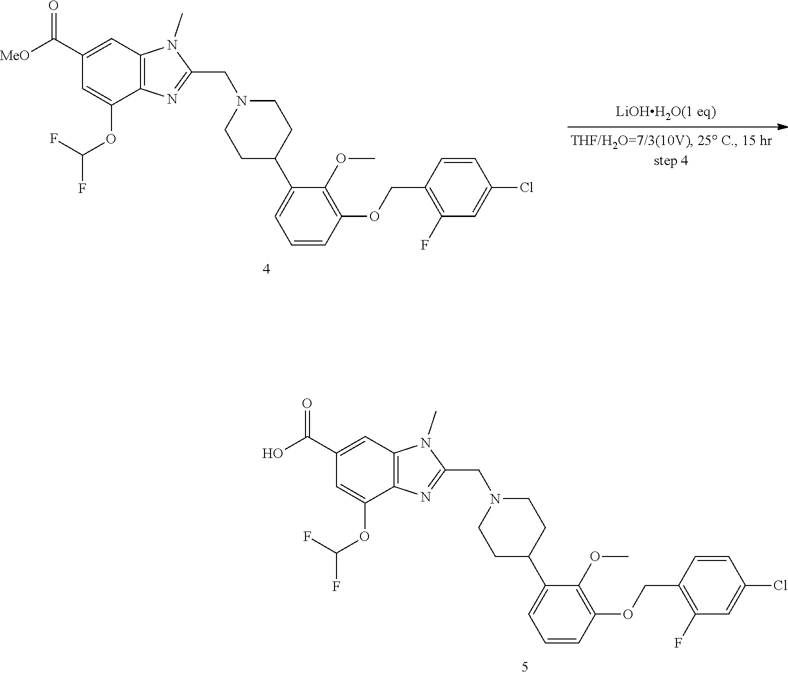 [SMILES: COC(=O)c1cc(OC(F)F)c2nc(CN3CCC(c4cccc(OCc5ccc(Cl)cc5F)c4OC)CC3)n(C)c2c1 ; COc1c(OCc2ccc(Cl)cc2F)cccc1C1CCN(Cc2nc3c(OC(F)F)cc(C(=O)O)cc3n2C)CC1]

To a solution of 4 (100 mg, 161.80 μmol, 1 eq) in THF (0.7 mL) and H₂O (0.3 mL) was added LiOH·H₂O (7.47 mg, 177.98 μmol, 1.1 eq). The mixture was stirred at 25° C. for 15 hr. LC-MS showed 4 was consumed completely and one main peak with desired mass was detected. The mixture was filtered and concentrated in vacuum. The residue was purified by prep-HPLC (column: Waters Xbridge Prep OBD C18 150*40 mm*10 um; mobile phase: [H₂O (10 mM NH₄HCO₃)-ACN]; gradient: 20%-70% B over 8.0 min). 2-((4-(3-((4-Chloro-2-fluorobenzyl)oxy)-2-methoxyphenyl)piperidin-1-yl)methyl)-4-(difluoromethoxy)-1-methyl-1H-benzo[d]imidazole-6-carboxylic acid (29.80 mg, 49.29 μmol, 31.05% yield) was obtained as a white solid.

LCMS: RT=2.827 min, MS cal.: 603.2, [M+H]⁺=604.2.

HPLC: RT=12.477 min, purity: 99.91%

¹H NMR (400 MHz, MeOH-d4) δ=8.09 (s, 1H), 7.68 (s, 1H), 7.55 (t, J=8.0 Hz, 1H), 7.27-7.25 (m, 1H), 7.24 (t, J=74.0 Hz, 1H), 7.03-6.98 (m, 1H), 6.94-6.91 (m, 1H), 6.89-6.86 (m, 1H), 5.12 (s, 2H), 4.02 (s, 3H), 3.97 (s, 2H), 3.80 (s, 3H), 3.09 (d, J=11.2 Hz, 2H), 3.05-2.95 (m, 1H), 2.45-2.35 (m, 2H), 1.83-1.74 (m, 4H)

### Example A174—Preparation of Compound 376 and Compound 377. (S)-2-((4-(6-((4-Cyano-2-fluorobenzyl)oxy)pyridin-2-yl)piperidin-1-yl)methyl)-4-hydroxy-1-(oxetan-2-ylmethyl)-1H-benzo[d]imidazole-5-carboxylic acid (Compound 376) (15) & (S)-2-((4-(6-((4-Cyano-2-fluorobenzyl)oxy)pyridin-2-yl)piperidin-1-yl)methyl)-7-hydroxy-1-(oxetan-2-ylmethyl)-1H-benzo[d]imidazole-6-carboxylic acid (Compound 377) (15A)

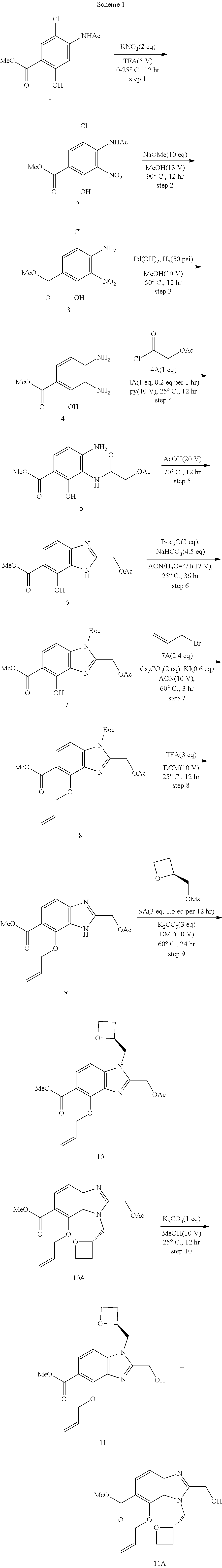

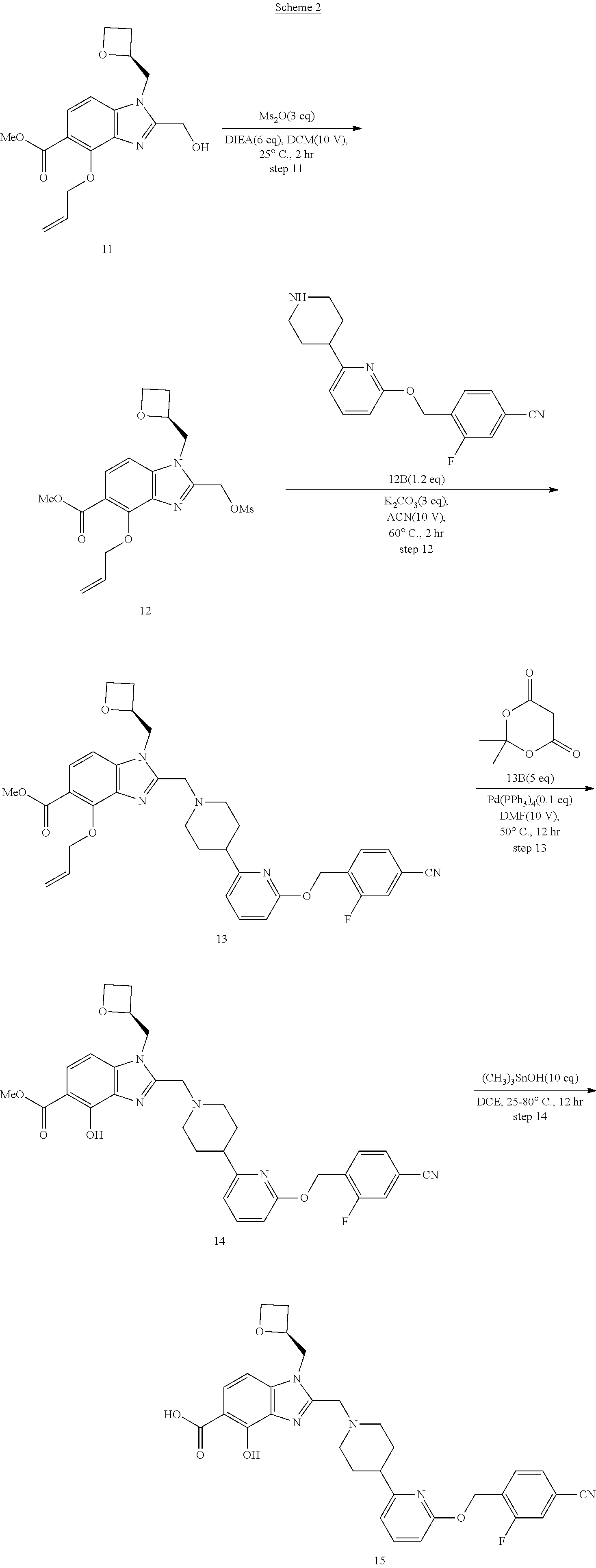

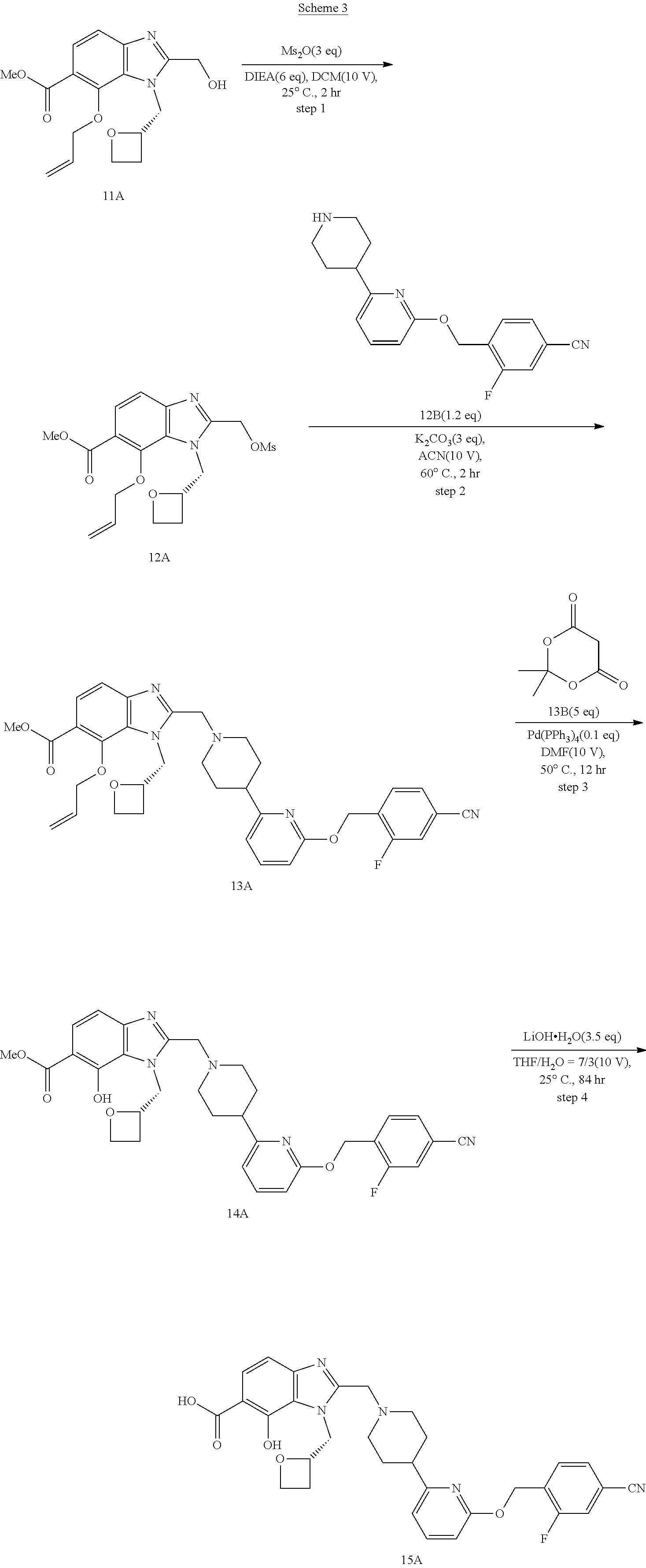

### General Procedure for Preparation of Scheme 1, Intermediate 2:

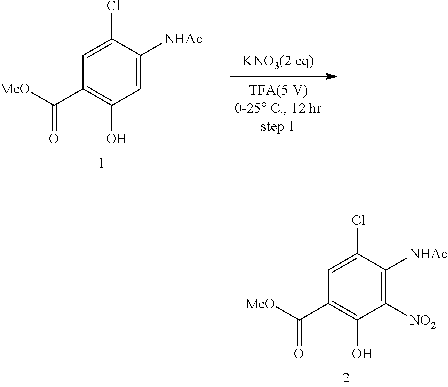 [SMILES: COC(=O)c1cc(Cl)c(NC(C)=O)cc1O ; COC(=O)c1cc(Cl)c(NC(C)=O)c([N+](=O)[O-])c1O]

TFA (200 mL) was charged to the three-necked round bottom flask (500 mL), then 1 (40 g, 164.17 mmol, 1 eq) was added to the mixture at 25° C. At 0° C., KNO₃ (33.20 g, 328.35 mmol, 2 eq) was added in portions to the reaction mixture at 0° C. within 10 min. After the addition, the mixture was stirred at 25° C. for 12 hr. TLC (Petroleum ether/Ethyl acetate=1/1, R_{f}=0.3) indicated 1 was consumed completely. The reaction mixture was dropwise added to H₂O (400 mL) at 0° C. within 30 min. The mixture was filtered to give crude product as a yellow solid. The crude product was triturated with PE/MTBE=1/1 (10 V) at 25° C. for 10 min. The mixture was filtered to give crude product as a yellow solid. The crude product was triturated with MeOH (1 V) at 25° C. for 10 min. Then the mixture was filtered, and the filter cake was Compound 2 (50 g, 173.23 mmol, 53% yield) obtained as a yellow solid.

LCMS: RT=0.528, MS cal.: 288.0; 290.0, [M−H]⁻=286.8

¹H NMR (400 MHz, DMSO-d₆) δ=11.18 (br s, 1H), 10.27 (br s, 1H), 8.05 (s, 1H), 3.92 (s, 3H), 2.03 (s, 3H)

KF: 0.08%

### General Procedure for Preparation of Scheme 1, Intermediate 3:

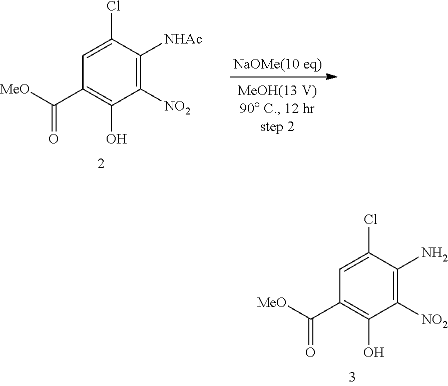 [SMILES: COC(=O)c1cc(Cl)c(NC(C)=O)c([N+](=O)[O-])c1O ; COC(=O)c1cc(Cl)c(N)c([N+](=O)[O-])c1O]

To a solution of 2 (15 g, 51.97 mmol, 1 eq) in MeOH (200 mL) was added NaOMe (28.07 g, 519.68 mmol, 10 eq) in portions at 0° C., and then the mixture was stirred at 90° C. for 12 hr. LCMS (RT=0.909 min) showed 2 was consumed completely. Two reactions were combined for workup. The reaction mixture was dropwise added to HCl (4 M, 210 mL) at 0° C. within 30 min. The mixture was filtered, and the filtered cake was crude product as a yellow solid. The crude product was triturated with MeOH (1 V) at 25° C. for 10 min. Then the mixture was filtered, and the filter cake was 3 (25 g, 101.38 mmol, 97% yield) obtained as a yellow solid.

LCMS: RT=0.899 min. MS cal.: 246.0; 248.0, [M+H]⁺=246.9

¹H NMR (400 MHz, DMSO-d₆) δ=11.51 (s, 1H), 7.76 (s, 1H), 7.08 (s, 2H), 3.86 (s, 3H)

### General Procedure for Preparation of Scheme 1, Intermediate 4:

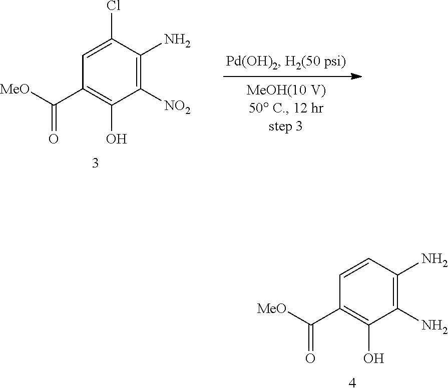 [SMILES: COC(=O)c1cc(Cl)c(N)c([N+](=O)[O-])c1O ; COC(=O)c1ccc(N)c(N)c1O]

MeOH (130 mL) was charged to the hydrogenating flask (500 mL), the mixture was degassed and purged with Ar for 3 times, then Pd(OH)₂ (7.12 g) was added to the mixture at 20° C. At 20° C. (inner temperature), 3 (12.5 g, 50.69 mmol, 1 eq) was added in portions to the reaction mixture at 20° C. After the addition, the mixture was stirred at 50° C. for 12 hr under H₂ (50 psi) atmosphere. LCMS (RT=0.784 min) showed 3 was consumed completely. The reaction mixture was filtered, the cake was washed three times with MeOH (2000 mL) and concentrated under reduced pressure to give a residue. 4 (9.2 g, 50.50 mmol, 99% yield) was obtained as a brown solid.

¹H NMR (400 MHz, DMSO-d₆) δ=11.34 (br s, 1H), 7.43 (d, J=8.8 Hz, 1H), 6.34 (d, J=8.8 Hz, 1H), 3.83 (s, 3H)

### General Procedure for Preparation of Scheme 1, Intermediate 5:

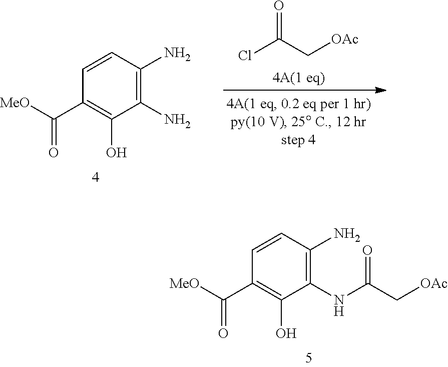 [SMILES: COC(=O)c1ccc(N)c(N)c1O ; CC(=O)OCC(=O)Cl ; COC(=O)c1ccc(N)c(NC(=O)COC(C)=O)c1O]

A mixture of 4 (5 g, 27.45 mmol, 1 eq) in Py (10 mL) was degassed and purged with N₂ for 3 times, the mixture was added 4A (3.75 g, 27.45 mmol, 2.95 mL, 1 eq, 0.2 eq per 1 hr). The mixture was stirred at 25° C. for 12 hr. LCMS (product RT=0.940 min) showed 18% of 4 remained. Several new peaks were shown on LCMS and 26% of desired Compound was detected. The reaction mixture was diluted with H₂O (50 mL) and extracted with DCM (50 mL*5). The combined organic layers were washed with brine (80 mL), dried over Na₂SO₄, filtered, and concentrated under reduced pressure to give a residue. The residue was purified by prep-HPLC (column: Phenomenex luna C₁₈ 250 mm*100 mm*15 um; mobile phase: [H₂O (0.2% FA)-ACN]; gradient: 10%-55% B over 30.0 min). 5 (1.7 g, 6.02 mmol, 22% yield) was obtained as a white solid.

¹H NMR (400 MHz, DMSO-d₆) δ=11.08 (s, 1H), 8.97 (s, 1H), 7.44 (d, J=8.8 Hz, 1H), 6.24 (d, J=8.8 Hz, 1H), 5.92 (s, 2H), 4.65 (s, 2H), 3.81 (s, 3H), 2.11 (s, 3H)

### General Procedure for Preparation of Scheme 1, Intermediate 6:

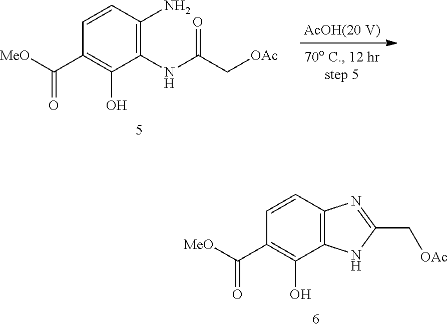 [SMILES: COC(=O)c1ccc(N)c(NC(=O)COC(C)=O)c1O ; COC(=O)c1ccc2nc(COC(C)=O)[nH]c2c1O]

A mixture of 5 (6.6 g, 23.38 mmol, 1 eq) in AcOH (132 mL) was degassed and purged with N₂ for 3 times, and then the mixture was stirred at 70° C. for 12 hr under N₂ atmosphere. LCMS (RT=0.854 min) showed 5 was consumed completely and one main peak with desired mass was detected. The reaction mixture was concentrated under reduced pressure to remove AcOH. The residue was diluted with H₂O (50 mL) and extracted with DCM (50 mL*4). The combined organic layers were dried over Na₂SO₄, filtered and concentrated under reduced pressure to give a residue. The crude product was triturated with H₂O (70 mL) at 25° C. for 15 min. Then the mixture was filtered, and the filter cake was 6 (5.9 g, 22.33 mmol, 95% yield) obtained as a brown solid.

LCMS: RT=0.851 min, MS cal.: 264.1, [M+H]⁺=264.9

¹H NMR (400 MHz, DMSO-d₆) δ=13.25-12.74 (m, 1H), 11.52-11.12 (m, 1H), 7.62 (d, J=8.8 Hz, 1H), 7.20-6.96 (m, 1H), 5.25 (s, 2H), 3.91 (s, 3H), 2.12 (s, 3H)

### General Procedure for Preparation of Scheme 1, Intermediate 7:

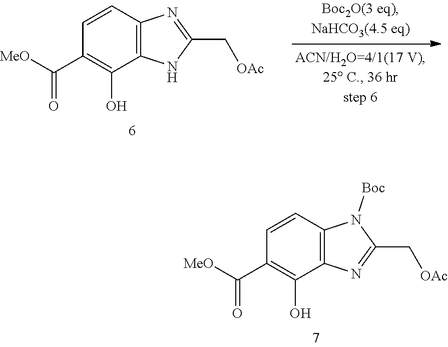 [SMILES: COC(=O)c1ccc2nc(COC(C)=O)[nH]c2c1O ; COC(=O)c1ccc2c(nc(COC(C)=O)n2C(=O)OC(C)(C)C)c1O]

A mixture of 6 (3 g, 11.35 mmol, 1 eq), Boc₂O (2.48 g, 11.35 mmol, 2.61 mL, 1 eq), NaHCO₃ (2.38 g, 28.38 mmol, 2.5 eq) in ACN (30 mL) and H₂O (10 mL) was degassed and purged with N₂ for 3 times, and then the mixture was stirred at 25° C. for 12 hr under N₂ atmosphere. Then the reaction mixture was added NaHCO₃ (953.78 mg, 11.35 mmol, 1 eq) and Boc₂O (2.48 g, 11.35 mmol, 2.61 mL, 1 eq), the resulting mixture was stirred at 25° C. for 12 hr. Then the reaction mixture was added NaHCO₃ (953.78 mg, 11.35 mmol, 1 eq) and Boc₂O (2.48 g, 11.35 mmol, 2.61 mL, 1 eq), the resulting mixture was stirred at 25° C. for 12 hr. LCMS (6 RT=0.849 min, 7 RT=1.264 min) showed 26% of 6 remained. Several new peaks were shown on LCMS and 63% of desired Compound was detected. The combined reaction mixture was diluted with water (30 mL) and extracted with DCM (40 mL*4). The combined organic phase was dried over Na₂SO₄, filtered, and concentrated under reduced pressure to give a residue. The residue was purified by column chromatography (SiO₂, Petroleum ether/Ethyl acetate=100/1 to 0/1). 7 (3.8 g, 7.61 mmol, 67% yield) was obtained as a white solid.

LCMS: RT=1.268, MS cal.: 364.4, [M+H]⁺=365.0

¹H NMR (400 MHz, DMSO-d₆) δ=11.19 (s, 1H), 7.82 (d, J=8.8 Hz, 1H), 7.51 (d, J=8.8 Hz, 1H), 5.45 (s, 2H), 3.93 (s, 3H), 2.20-2.13 (m, 3H), 1.64 (s, 9H)

### General Procedure for Preparation of Scheme 1, Intermediate 8:

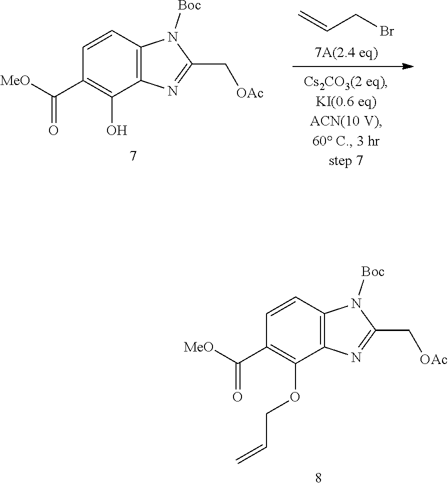

A mixture of 7 (1.2 g, 3.29 mmol, 1 eq), 7A (956.25 mg, 7.90 mmol, 2.4 eq), Cs₂CO₃ (2.15 g, 6.59 mmol, 2 eq), KI (328.04 mg, 1.98 mmol, 0.6 eq) in ACN (12 mL) was degassed and purged with N₂ for 3 times, and then the mixture was stirred at 60° C. for 3 hr under N₂ atmosphere. LCMS (product RT=1.372 min) showed 2 was consumed completely. The combined reaction mixture was diluted with water (20 mL) and extracted with DCM (20 mL*4). The combined organic phase was dried over Na₂SO₄, filtered, and concentrated under reduced pressure to give a residue. The residue was purified by column chromatography (SiO₂, Petroleum ether/Ethyl acetate=100/1 to 0/1). 8 (1.1 g, 2.56 mmol, 77% yield, 94% purity) was obtained as a white solid.

LCMS: RT=1.377 min. MS cal.: 404.4, [M+H]⁺=305.0

¹H NMR (400 MHz, DMSO-d₆) δ=7.75-7.62 (m, 2H), 6.08 (tdd, J=5.2, 10.6, 17.2 Hz, 1H), 5.47 (s, 2H), 5.43-5.35 (m, 1H), 5.19 (dd, J=1.6, 10.4 Hz, 1H), 5.16-5.08 (m, 2H), 3.82 (s, 3H), 2.23-2.10 (m, 3H), 1.65 (s, 9H)

### General Procedure for Preparation of Scheme 1, Intermediate 9:

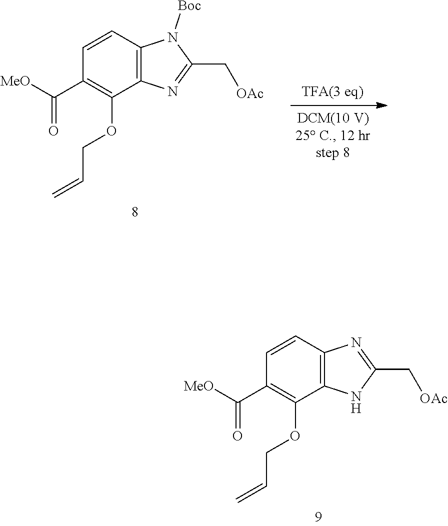 [SMILES: C=CCOc1c(C(=O)OC)ccc2c1nc(COC(C)=O)n2C(=O)OC(C)(C)C ; C=CCOc1c(C(=O)OC)ccc2nc(COC(C)=O)[nH]c12]

A mixture of 8 (1 g, 2.47 mmol, 1 eq), TFA (845.84 mg, 7.42 mmol, 551.04 μL, 3 eq) in DCM (10 mL) was degassed and purged with N₂ for 3 times, and then the mixture was stirred at 25° C. for 12 hr under N₂ atmosphere. LCMS (product RT=0.937 min) showed 8 was consumed completely and one main peak with desired m/z was detected. The reaction mixture was added to saturated NaHCO₃ (10 mL) at 0° C., and then diluted with H₂O 5 mL and extracted with DCM (10 mL*3). The combined organic layers were dried over Na₂SO₄, filtered, and concentrated under reduced pressure to give a residue. 9 (780 mg, 2.33 mmol, 94% yield) was obtained as a white oil.

LCMS: RT=0.933 min, MS cal.: 304.3, [M+H]⁺=305.0

¹H NMR (400 MHz, MeOH-d4) δ=7.70 (dd, J=4.8, 8.4 Hz, 1H), 7.23 (d, J=5.2 Hz, 1H), 6.25-6.10 (m, 1H), 5.38 (d, J=17.6 Hz, 1H), 5.33 (s, 2H), 5.20 (d, J=9.6 Hz, 1H), 5.03 (s, 2H), 3.89 (s, 3H), 2.17 (s, 3H)

### General Procedure for Preparation of Scheme 1, Intermediate 10 & Intermediate 10A:

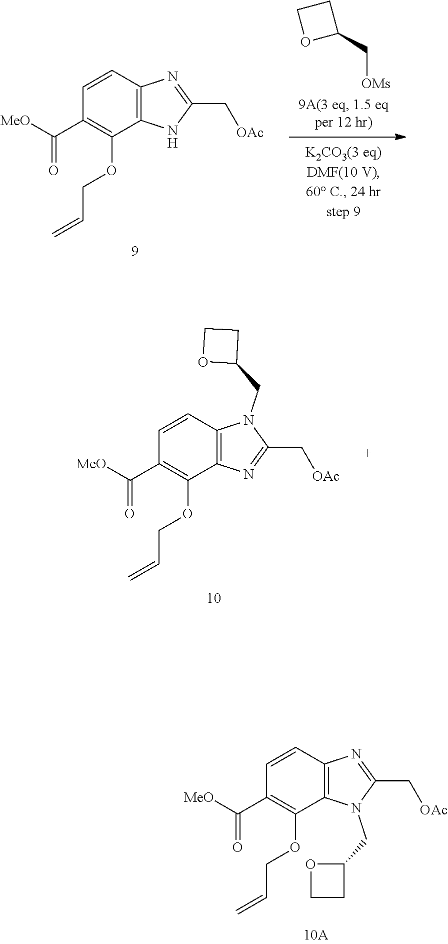 [SMILES: C=CCOc1c(C(=O)OC)ccc2nc(COC(C)=O)[nH]c12 ; CS(=O)(=O)OC[C@@H]1CCO1 ; C=CCOc1c(C(=O)OC)ccc2c1nc(COC(C)=O)n2C[C@@H]1CCO1 ; C=CCOc1c(C(=O)OC)ccc2nc(COC(C)=O)n(C[C@@H]3CCO3)c12]

A mixture of 9 (780 mg, 2.56 mmol, 1 eq), 9A (639.01 mg, 3.84 mmol, 1.5 eq), K₂CO₃ (1.06 g, 7.69 mmol, 3 eq) in DMF (7.5 mL) was degassed and purged with N₂ for 3 times, and then the mixture was stirred at 60° C. for 12 hr under N₂ atmosphere. Then the mixture was added 9A (639.01 mg, 3.84 mmol, 1.5 eq), the mixture was stirred at 60° C. for 12 hr under N₂ atmosphere. LCMS (9 RT=0.943 min, product RT=1.034 min) showed 4% of 9 remained. Several new peaks were shown on LC-MS and 40% of desired m/z was detected. The reaction mixture was filtered, the cake was washed by DCM (50 mL), and the filtrate was concentrated under reduced pressure to give a residue. The residue was diluted with brine 20 mL and extracted with DCM (20 mL*3). The combined organic layers were washed with brine (20 mL*2), dried over Na₂SO₄, filtered, and concentrated under reduced pressure to give a residue. A mixture of Intermediate 10 & 10A (950 mg, crude) was obtained as a yellow oil.

### General Procedure for Preparation of Scheme 1, Intermediate 11 & Intermediate 11A:

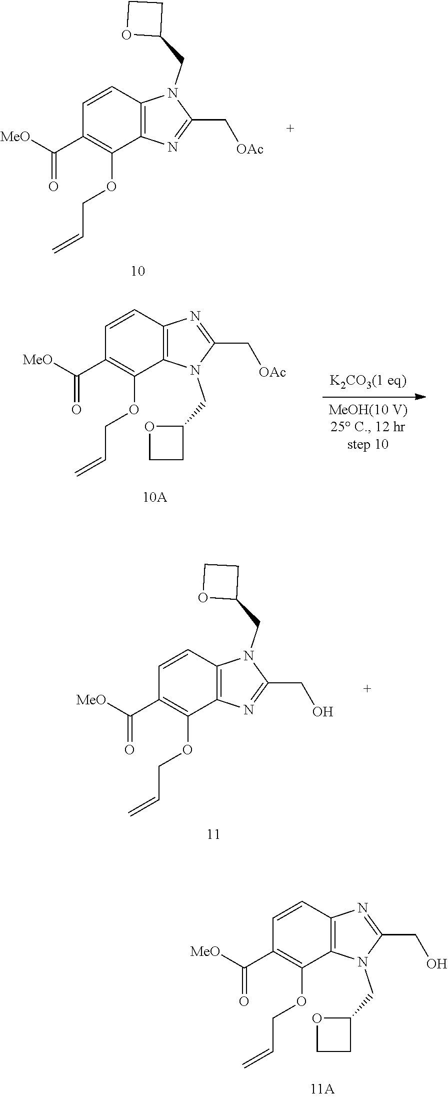 [SMILES: C=CCOc1c(C(=O)OC)ccc2c1nc(COC(C)=O)n2C[C@@H]1CCO1 ; C=CCOc1c(C(=O)OC)ccc2nc(COC(C)=O)n(C[C@@H]3CCO3)c12 ; C=CCOc1c(C(=O)OC)ccc2c1nc(CO)n2C[C@@H]1CCO1 ; C=CCOc1c(C(=O)OC)ccc2nc(CO)n(C[C@@H]3CCO3)c12]

A mixture of Intermediate 10 & 10A (950 mg, 2.54 mmol, 1 eq), K₂CO₃ (350.69 mg, 2.54 mmol, 1 eq) in MeOH (9.5 mL) was degassed and purged with N₂ for 3 times, and then the mixture was stirred at 25° C. for 12 hr under N₂ atmosphere. TLC (Petroleum ether/Ethyl acetate=0/1, 11 R_{f}=0.43, 11A R_{f}=0.24) indicated Intermediate 10 & 10 A was consumed, and two major new spots with larger polarity was detected. The reaction mixture was diluted with H₂O 10 mL and extracted with DCM (10 mL*4). The combined organic layers were dried over Na₂SO₄, filtered, and concentrated under reduced pressure to give a residue. The residue was purified by column chromatography (SiO₂, Petroleum ether/Ethyl acetate=100/1 to Ethyl acetate/MeOH=0/1). 11 (550 mg, 1.52 mmol, 60% yield) was obtained as a white solid.

LCMS: RT=0.900 min, MS cal.: 332.4, [M+H]⁺=333.0

¹H NMR (400 MHz, DMSO-d₆) δ=7.56 (d, J=8.8 Hz, 1H), 7.35 (d, J=8.8 Hz, 1H), 6.08 (dd, J=6.0, 11.2 Hz, 1H), 5.75 (s, 1H), 5.64 (t, J=6.0 Hz, 1H), 5.43 (dd, J=1.6, 17.2 Hz, 1H), 5.25-5.16 (m, 3H), 5.05 (dq, J=2.8, 7.2 Hz, 1H), 4.81-4.71 (m, 2H), 4.70-4.61 (m, 1H), 4.54-4.43 (m, 2H), 4.36 (td, J=6.0, 9.2 Hz, 1H), 3.79 (s, 3H), 2.73-2.62 (m, 1H), 2.45-2.35 (m, 1H). 11A (40 mg, 105.91 μmol, 4% yield) was obtained as a white solid.

LCMS: RT=0.911 min, MS cal.: 332.4, [M+H]⁺=333.0

¹H NMR (400 MHz, DMSO-d₆) δ=7.62 (d, J=8.4 Hz, 1H), 7.42 (d, J=8.4 Hz, 1H), 6.07 (d, J=6.8 Hz, 1H), 5.66 (t, J=6.0 Hz, 1H), 5.41 (dd, J=1.6, 17.2 Hz, 1H), 5.28 (dd, J=1.2, 10.4 Hz, 1H), 5.03 (dq, J=2.8, 7.2 Hz, 1H), 4.88-4.79 (m, 2H), 4.75 (d, J=5.2 Hz, 1H), 4.63-4.55 (m, 3H), 4.48 (d, J=7.2 Hz, 1H), 4.37 (td, J=6.0, 8.8 Hz, 1H), 3.84 (s, 3H), 2.71-2.60 (m, 1H), 2.46-2.33 (m, 1H)

### General Procedure for Preparation of Scheme 2, Intermediate 12:

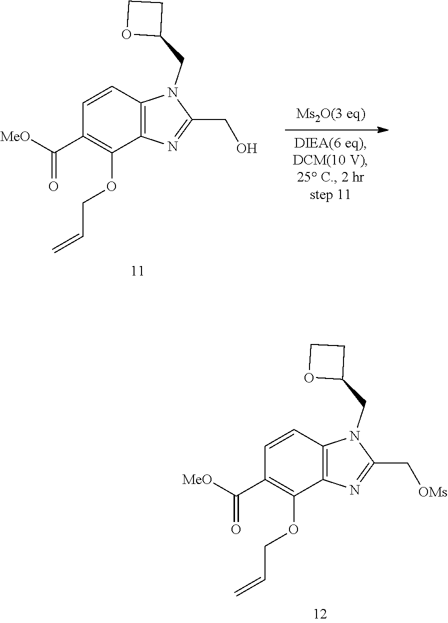 [SMILES: C=CCOc1c(C(=O)OC)ccc2c1nc(CO)n2C[C@@H]1CCO1 ; C=CCOc1c(C(=O)OC)ccc2c1nc(COS(C)(=O)=O)n2C[C@@H]1CCO1]

A mixture of 11 (250 mg, 752.22 μmol, 1 eq), Ms₂O (393.10 mg, 2.26 mmol, 3 eq), DIEA (583.31 mg, 4.51 mmol, 786.14 μL, 6 eq) in DCM (3 mL) was degassed and purged with N₂ for 3 times, and then the mixture was stirred at 25° C. for 2 hr under N₂ atmosphere. LCMS (RT=1.050 min) showed 11 was consumed completely and one main peak with desired mass was detected. The reaction mixture was added HCOOH at 25° C. until pH=7-8, and then diluted with H₂O 10 mL and extracted with DCM (10 mL*4). The combined organic layers were dried over Na₂SO₄, filtered, and concentrated under reduced pressure to give a residue. 12 (308 mg, 675.37 μmol, 89% yield) was obtained as a yellow oil.

LCMS: RT=0.418 min, MS cal.: 410.4, [M+H]⁺=411.0

### General Procedure for Preparation of Scheme 2, Intermediate 13:

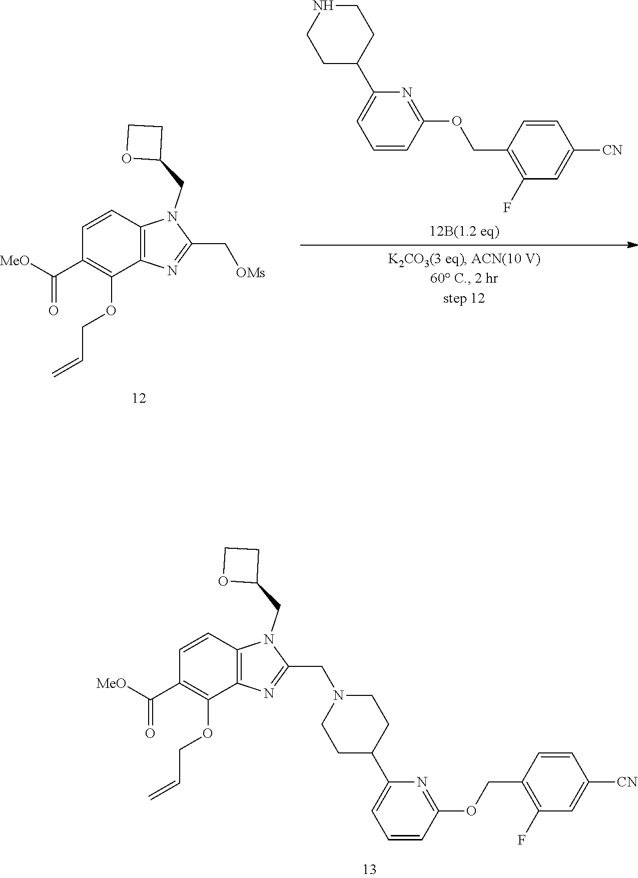 [SMILES: C=CCOc1c(C(=O)OC)ccc2c1nc(COS(C)(=O)=O)n2C[C@@H]1CCO1 ; N#Cc1ccc(COc2cccc(C3CCNCC3)n2)c(F)c1 ; C=CCOc1c(C(=O)OC)ccc2c1nc(CN1CCC(c3cccc(OCc4ccc(C#N)cc4F)n3)CC1)n2C[C@@H]1CCO1]

A mixture of 12 (308 mg, 750.41 μmol, 1 eq), 12B (280.37 mg, 900.50 μmol, 1.2 eq), K₂CO₃ (311.13 mg, 2.25 mmol, 3 eq) in ACN (3 mL) was degassed and purged with N₂ for 3 times, and then the mixture was stirred at 60° C. for 2 hr under N₂ atmosphere. LCMS (RT=1.550 min) showed 12 was consumed. Several new peaks were shown on LCMS and 67% of desired Compound was detected. The reaction mixture was diluted with H₂O 10 mL and extracted with DCM (10 mL*4). The combined organic layers were dried over Na₂SO₄, filtered, and concentrated under reduced pressure to give a residue. The residue was purified by column chromatography (SiO₂, Petroleum ether/Ethyl acetate=100/1 to Ethyl acetate/Methanol=0/1). 13 (300 mg, 393.17 μmol, 52% yield) was obtained as a yellow oil.

LCMS: RT=1.572 min, MS cal.: 625.7, [M+H]⁺=626.1

¹H NMR (400 MHz, DMSO-d6) δ=7.88 (d, J=10.4 Hz, 1H), 7.70 (d, J=3.6 Hz, 2H), 7.65 (t, J=7.6 Hz, 1H), 7.55 (d, J=8.4 Hz, 1H), 7.35 (d, J=8.4 Hz, 1H), 6.89 (d, J=7.2 Hz, 1H), 6.72 (d, J=8.0 Hz, 1H), 6.20-6.00 (m, 1H), 5.47 (s, 2H), 5.46-5.37 (m, 1H), 5.23 (d, J=5.2 Hz, 2H), 5.18 (dd, J=1.6, 10.4 Hz, 1H), 5.10 (dt, J=4.4, 7.2 Hz, 1H), 4.77-4.70 (m, 1H), 4.63-4.56 (m, 1H), 4.51-4.44 (m, 1H), 4.40 (td, J=6.0, 8.8 Hz, 1H), 3.92 (d, J=13.6 Hz, 1H), 3.85-3.78 (m, 3H), 3.75 (s, 1H), 2.97 (d, J=10.4 Hz, 1H), 2.84 (d, J=10.8 Hz, 1H), 2.74-2.66 (m, 1H), 2.63-2.54 (m, 1H), 2.44-2.38 (m, 1H), 2.30-2.14 (m, 2H), 1.84-1.58 (m, 4H

### General Procedure for Preparation of Scheme 2, Intermediate 14:

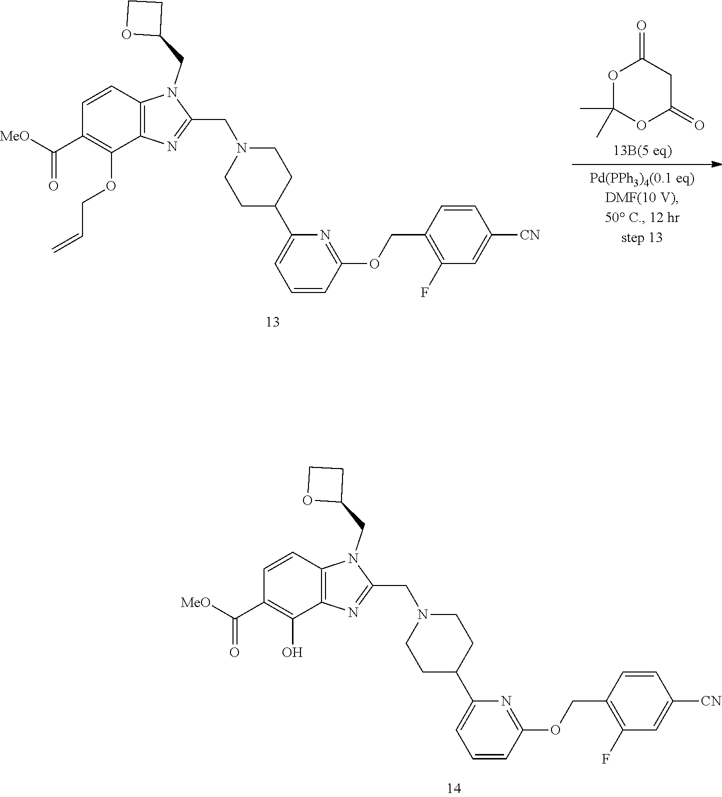

A mixture of 13 (200 mg, 319.65 μmol, 1 eq), Pd(PPh₃)₄ (36.94 mg, 31.96 μmol, 0.1 eq), 13B (230.35 mg, 1.60 mmol, 5 eq) in DMF (2 mL) was degassed and purged with N₂ for 3 times, and then the mixture was stirred at 50° C. for 12 hr under N₂ atmosphere. LCMS (RT=0.433 min) showed 13 was consumed completely and one main peak with desired mass was detected. The reaction mixture was filtered. The combined filtrate was purified by prep-HPLC (column: Waters Xbridge Prep OBD C₁₈ 150*40 mm*10 um; mobile phase: [H₂O (10 mM NH₄HCO₃)-ACN]; gradient: 45%-75% B over 8.0 min). 14 (160 mg, 248.62 μmol, 77% yield) was obtained as a pink solid.

LCMS: RT=1.462 min, MS cal.: 585.6, [M+H]⁺=586.1

¹H NMR (400 MHz, DMSO-d₆) δ=11.34-10.98 (m, 1H), 7.87 (d, J=10.0 Hz, 1H), 7.69 (d, J=3.6 Hz, 2H), 7.67-7.61 (m, 2H), 7.20 (d, J=8.8 Hz, 1H), 6.88 (d, J=7.2 Hz, 1H), 6.72 (d, J=8.0 Hz, 1H), 5.46 (s, 2H), 5.09 (dq, J=3.2, 7.2 Hz, 1H), 4.76-4.67 (m, 1H), 4.63-4.55 (m, 1H), 4.50-4.43 (m, 1H), 4.37 (td, J=6.0, 8.8 Hz, 1H), 3.96-3.86 (m, 4H), 3.79-3.72 (m, 1H), 2.96 (d, J=11.2 Hz, 1H), 2.83 (d, J=11.2 Hz, 1H), 2.73-2.65 (m, 1H), 2.62-2.55 (m, 1H), 2.45-2.37 (m, 1H), 2.25-2.11 (m, 2H), 1.80-1.58 (m, 4H)

### General Procedure for Preparation of Scheme 2, Compound 376 (15):

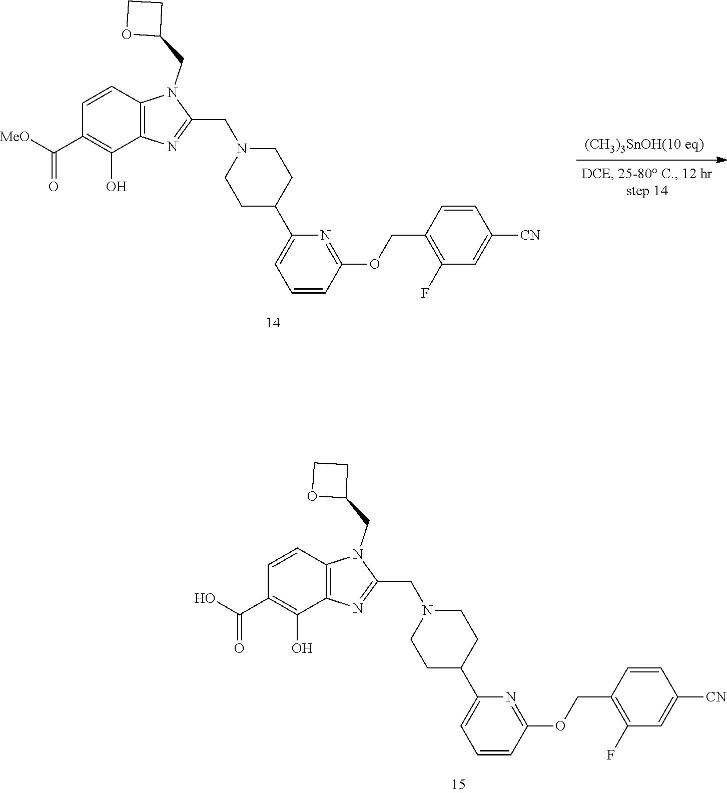 [SMILES: COC(=O)c1ccc2c(nc(CN3CCC(c4cccc(OCc5ccc(C#N)cc5F)n4)CC3)n2C[C@@H]2CCO2)c1O ; N#Cc1ccc(COc2cccc(C3CCN(Cc4nc5c(O)c(C(=O)O)ccc5n4C[C@@H]4CCO4)CC3)n2)c(F)c1]

DCE (0.6 mL) was charged to the thumb bottle, then 14 (60 mg, 102.45 μmol, 1 eq) was added to the mixture at 25° C. At 25° C. (inner temperature), (CH₃)₃SnOH (185.26 mg, 1.02 mmol, 10 eq) was added to the reaction mixture at 25° C. After the addition, the mixture was stirred at 80° C. for 12 hr. LCMS (RT=1.082 min) showed 14 was consumed completely and one main peak with desired mass was detected. The reaction mixture was added saturated KF solution (3 mL) at 25° C., the mixture was diluted with H₂O (5 mL) and extracted with DCM (10 mL*4). The combined organic layers were dried over Na₂SO₄, filtered, and concentrated under reduced pressure to give a residue. The combined reaction mixture was filtered. The filtrate was purified by prep-HPLC (column: Waters Xbridge Prep OBD C₁₈ 150*40 mm*10 um; mobile phase: [H₂O (10 mM NH₄HCO₃)-ACN]; gradient: 5%-45% B over 8.0 min). The residue was repurified by prep-HPLC (column: Phenomenex Luna C₁₈ 100*30 mm*3 um; mobile phase: [H₂O (0.2% FA)-ACN]; gradient: 15%-50% B over 8.0 min). (S)-2-((4-(6-((4-Cyano-2-fluorobenzyl)oxy)pyridin-2-yl)piperidin-1-yl)methyl)-4-hydroxy-1-(oxetan-2-ylmethyl)-1H-benzo[d]imidazole-5-carboxylic acid (29.46 mg, 50.65 μmol, 24% yield, 98.27% purity) was obtained as a white solid.

LCMS: RT=2.152 min, MS cal.: 571.6, [M+H]⁺=572.3

HPLC: RT=10.434 min, purity: 98.27%

¹H NMR (400 MHz, MeOH-d4) δ=7.82 (d, J=8.8 Hz, 1H), 7.70-7.61 (m, 2H), 7.61-7.52 (m, 2H), 6.97 (d, J=8.8 Hz, 1H), 6.89 (d, J=7.2 Hz, 1H), 6.73 (d, J=8.0 Hz, 1H), 5.53 (s, 2H), 5.22 (br d, J=5.6 Hz, 1H), 4.73-4.62 (m, 2H), 4.61-4.54 (m, 1H), 4.48-4.37 (m, 3H), 3.60-3.47 (m, 2H), 3.02-2.91 (m, 2H), 2.91-2.84 (m, 1H), 2.81-2.72 (m, 1H), 2.55-2.45 (m, 1H), 2.03 (br d, J=3.6 Hz, 4H)

### General Procedure for Preparation of Scheme 3, Intermediate 12A:

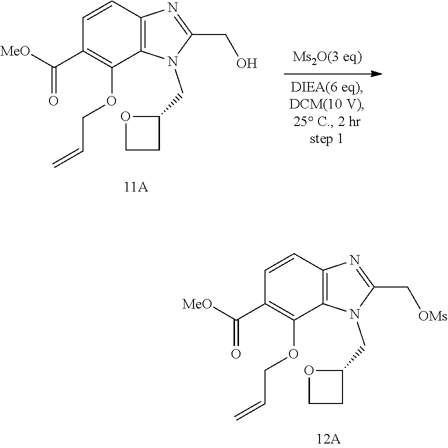 [SMILES: C=CCOc1c(C(=O)OC)ccc2nc(CO)n(C[C@@H]3CCO3)c12 ; C=CCOc1c(C(=O)OC)ccc2nc(COS(C)(=O)=O)n(C[C@@H]3CCO3)c12]

A mixture of 11A (40 mg, 120.35 μmol, 1 eq), Ms₂O (62.90 mg, 361.06 μmol, 3 eq), DIEA (93.33 mg, 722.13 μmol, 125.78 μL, 6 eq) in DCM (1 mL) was degassed and purged with N₂ for 3 times, and then the mixture was stirred at 25° C. for 2 hr under N₂ atmosphere. TLC (Petroleum ether/Ethyl acetate=0/1, R_{f}=0.69) indicated 11A was consumed, and one major new spot with lower polarity was detected. The reaction mixture was added HCOOH at 25° C. until pH=7-8, and then diluted with H₂O 7 mL and extracted with DCM (7 mL*4). The combined organic layers were dried over Na₂SO₄, filtered, and concentrated under reduced pressure to give a residue. 12A (49 mg, crude) was obtained as a yellow oil.

### General Procedure for Preparation of Scheme 3, Intermediate 13A:

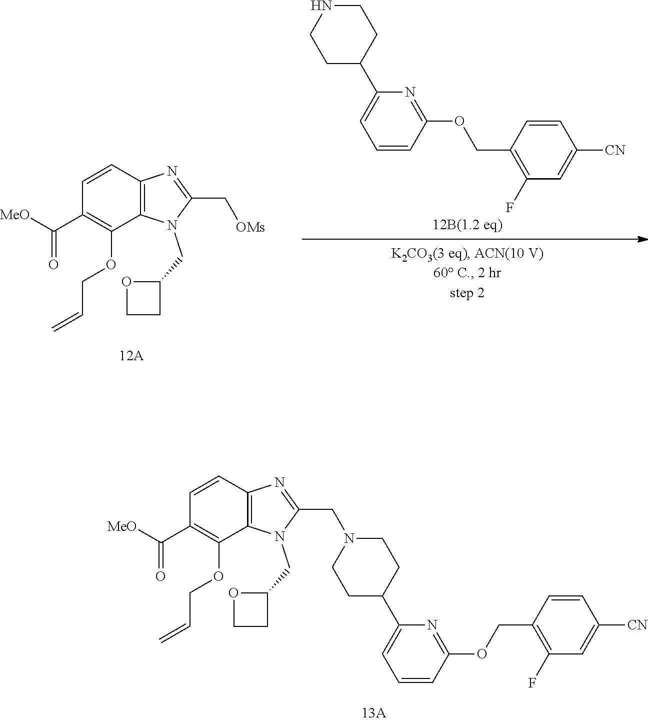 [SMILES: C=CCOc1c(C(=O)OC)ccc2nc(COS(C)(=O)=O)n(C[C@@H]3CCO3)c12 ; N#Cc1ccc(COc2cccc(C3CCNCC3)n2)c(F)c1 ; C=CCOc1c(C(=O)OC)ccc2nc(CN3CCC(c4cccc(OCc5ccc(C#N)cc5F)n4)CC3)n(C[C@@H]3CCO3)c12]

A mixture of 12A (49 mg, 119.38 μmol, 1 eq), 4B (44.60 mg, 143.26 μmol, 1.2 eq), K₂CO₃ (49.50 mg, 358.15 μmol, 3 eq) in ACN (1 mL) was degassed and purged with N₂ for 3 times, and then the mixture was stirred at 60° C. for 2 hr under N₂ atmosphere. LCMS (RT=1.515 min) showed 12A was consumed. Several new peaks were shown on LCMS and 63% of desired Compound was detected. The reaction mixture was diluted with H₂O 5 mL and extracted with DCM (5 mL*3). The combined organic layers were dried over Na₂SO₄, filtered, and concentrated under reduced pressure to give a residue. The residue was purified by prep-TLC (SiO₂, Petroleum ether/Ethyl acetate=0/1). 13A (60 mg, 76.72 μmol, 64% yield) was obtained as a yellow oil.

LCMS: RT=1.519 min, MS cal.: 625.7, [M+H]⁺=626.1

¹H NMR (400 MHz, DMSO-d₆) δ=7.86 (d, J=10.4 Hz, 1H), 7.70 (d, J=2.4 Hz, 2H), 7.65 (t, J=7.6 Hz, 1H), 7.60 (d, J=8.4 Hz, 1H), 7.41 (d, J=8.4 Hz, 1H), 6.89 (d, J=7.2 Hz, 1H), 6.72 (d, J=8.0 Hz, 1H), 6.13-6.02 (m, 1H), 5.49-5.36 (m, 3H), 5.27 (d, J=10.4 Hz, 1H), 5.08 (dd, J=2.0, 7.2 Hz, 1H), 5.03-4.92 (m, 1H), 4.68 (dd, J=3.2, 14.8 Hz, 1H), 4.59 (d, J=5.2 Hz, 2H), 4.52-4.45 (m, 1H), 4.39 (td, J=6.4, 8.8 Hz, 1H), 4.07-3.96 (m, 2H), 3.84 (s, 3H), 3.79-3.68 (m, 1H), 3.04-2.97 (m, 1H), 2.93-2.85 (m, 1H), 2.70-2.64 (m, 1H), 2.64-2.57 (m, 1H), 2.45-2.39 (m, 1H), 2.38-2.10 (m, 2H), 1.84-1.60 (m, 4H)

### General Procedure for Preparation of Scheme 3, Intermediate 14A:

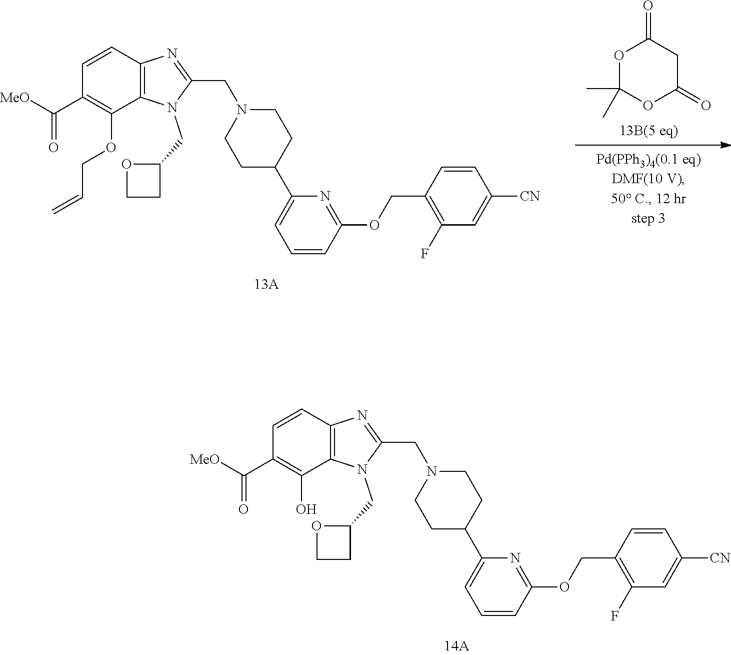

A mixture of 13A (60 mg, 95.89 μmol, 1 eq), Pd(PPh₃)₄ (11.08 mg, 9.59 μmol, 0.1 eq), 13B (69.10 mg, 479.47 μmol, 5 eq) in DMF (1 mL) was degassed and purged with N₂ for 3 times, and then the mixture was stirred at 60° C. for 12 hr under N₂ atmosphere. LCMS (RT=1.558 min) showed 13A was consumed completely and one main peak with desired mass was detected. The reaction mixture was filtered. The filtrate was purified by prep-HPLC (column: Waters Xbridge BEH C₁₈ 100*25 mm*10 um; mobile phase: [H₂O (10 mM NH₄HCO₃)-ACN]; gradient: 55%-85% B over 8.0 min). 14A (27 mg, 46.10 μmol, 48% yield) was obtained as a pink solid.

LCMS: RT=1.556 min, MS cal.: 585.6, [M+H]⁺=586.1

¹H NMR (400 MHz, DMSO-d₆) δ=11.55 (br s, 1H), 7.87 (d, J=10.0 Hz, 1H), 7.73-7.68 (m, 2H), 7.64 (t, J=7.6 Hz, 1H), 7.59 (d, J=8.8 Hz, 1H), 7.15 (d, J=8.8 Hz, 1H), 6.88 (d, J=7.2 Hz, 1H), 6.72 (d, J=8.0 Hz, 1H), 5.46 (s, 2H), 5.16-5.09 (m, 1H), 5.05-4.97 (m, 1H), 4.82 (dd, J=3.2, 14.4 Hz, 1H), 4.52-4.45 (m, 1H), 4.38 (td, J=6.0, 8.8 Hz, 1H), 3.93 (s, 4H), 3.80 (d, J=13.6 Hz, 1H), 2.96 (d, J=11.2 Hz, 1H), 2.87 (d, J=11.6 Hz, 1H), 2.75-2.66 (m, 1H), 2.58 (dd, J=3.6, 7.6 Hz, 1H), 2.47-2.41 (m, 1H), 2.28-2.14 (m, 2H), 1.82-1.63 (m, 4H)

### General Procedure for Preparation of Compound 377 (15A):

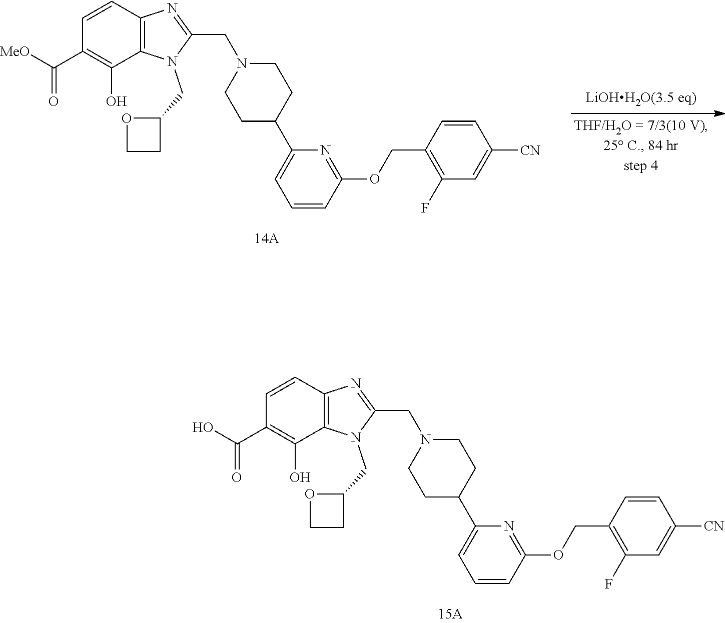 [SMILES: COC(=O)c1ccc2nc(CN3CCC(c4cccc(OCc5ccc(C#N)cc5F)n4)CC3)n(C[C@@H]3CCO3)c2c1O ; N#Cc1ccc(COc2cccc(C3CCN(Cc4nc5ccc(C(=O)O)c(O)c5n4C[C@@H]4CCO4)CC3)n2)c(F)c1]

A mixture of 14A (27 mg, 46.10 μmol, 1 eq), LiOH·H₂O (4.84 mg, 115.26 μmol, 2.5 eq) in H₂O (0.3 mL) and THF (0.7 mL) was degassed and purged with N₂ for 3 times, and then the mixture was stirred at 25° C. for 12 hr under N₂ atmosphere. The mixture was added LiOH·H₂O (967.36 μg, 23.05 μmol, 0.5 eq), then the mixture was stirred at 25° C. for 12 hr under N₂ atmosphere. The mixture was added LiOH·H₂O (967.36 μg, 23.05 μmol, 0.5 eq), then the mixture was stirred at 25° C. for 60 hr under N₂ atmosphere. LCMS (14A RT=1.558 min, product RT=1.092 min) showed 27% of 14A remained. Several new peaks were shown on LCMS and 23% of desired Compound was detected. The reaction mixture was filtered. The filtrate was purified by prep-HPLC (column: Waters Xbridge BEH C₁₈ 100*30 mm*10 um; mobile phase: [H₂O (10 mM NH₄HCO₃)-ACN]; gradient: 25%-55% B over 8.0 min). (S)-2-((4-(6-((4-Cyano-2-fluorobenzyl)oxy)pyridin-2-yl)piperidin-1-yl)methyl)-7-hydroxy-1-(oxetan-2-ylmethyl)-1H-benzo[d]imidazole-6-carboxylic acid (1.44 mg, 2.33 μmol, 5% yield, 92.64% purity) was obtained as a white solid.

LCMS: RT=2.326 min, MS cal.: 571.6, [M+H]⁺=572.3

HPLC: RT=10.413 min, purity: 92.64%

¹H NMR (400 MHz, MeOH-d4) δ=7.79 (d, J=8.8 Hz, 1H), 7.71-7.66 (m, 1H), 7.64 (t, J=7.6 Hz, 1H), 7.61-7.51 (m, 2H), 7.07 (d, J=8.8 Hz, 1H), 6.89 (d, J=7.2 Hz, 1H), 6.73 (d, J=8.4 Hz, 1H), 5.53 (s, 2H), 5.36-5.29 (m, 1H), 5.07-5.01 (m, 1H), 4.95 (d, J=2.8 Hz, 1H), 4.72-4.66 (m, 1H), 4.49-4.37 (m, 3H), 3.48 (d, J=3.6 Hz, 2H), 2.98-2.73 (m, 4H), 2.67-2.55 (m, 1H), 2.07-1.95 (m, 4H)

### Example A175—Preparation of Compound 176 and Compound 175. (S)-2-((4-(2-(4-Chloro-2-fluorophenyl)-2H-chromen-8-yl)piperidin-1-yl)methyl)-4-(difluoromethoxy)-1-methyl-1H-benzo[d]imidazole-6-carboxylic acid (Compound 176) (10A) & (R)-2-((4-(2-(4-Chloro-2-fluorophenyl)-2H-chromen-8-yl)piperidin-1-yl)methyl)-4-(difluoromethoxy)-1-methyl-1H-benzo[d]imidazole-6-carboxylic acid (Compound 175) (10B)

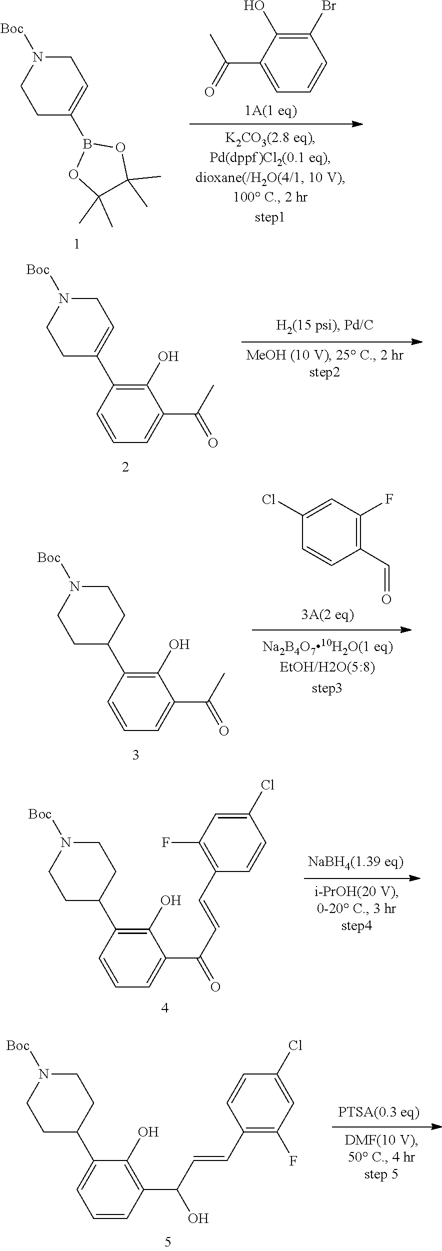

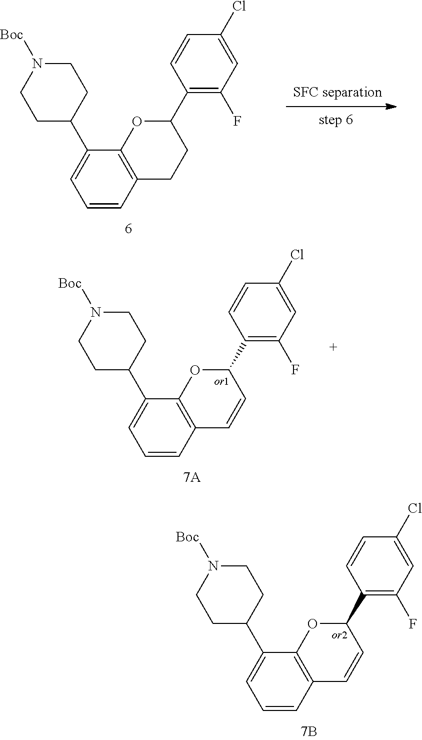 [SMILES: CC(C)(C)OC(=O)N1CCC(c2cccc3c2OC(c2ccc(Cl)cc2F)CC3)CC1 ; CC(C)(C)OC(=O)N1CCC(c2cccc3c2O[C@H](c2ccc(Cl)cc2F)C=C3)CC1 ; CC(C)(C)OC(=O)N1CCC(c2cccc3c2O[C@@H](c2ccc(Cl)cc2F)C=C3)CC1]

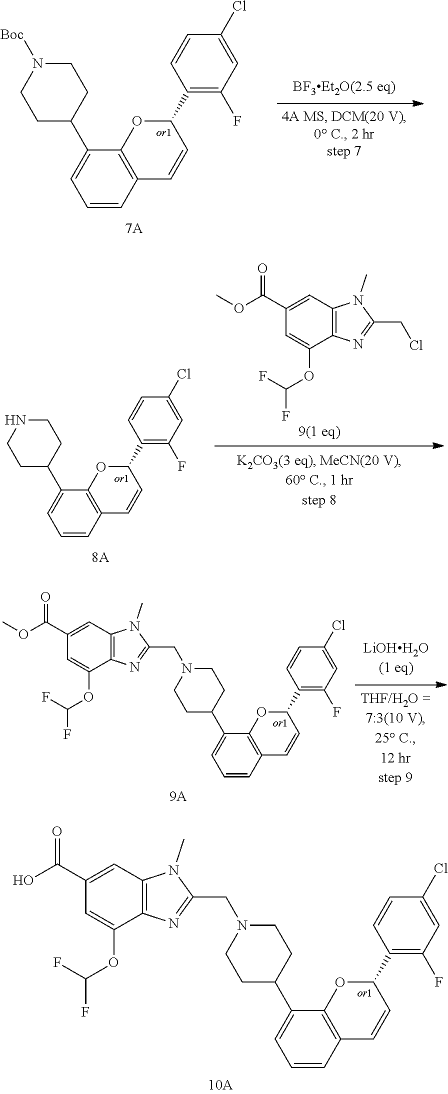 [SMILES: CC(C)(C)OC(=O)N1CCC(c2cccc3c2O[C@H](c2ccc(Cl)cc2F)C=C3)CC1 ; Fc1cc(Cl)ccc1[C@@H]1C=Cc2cccc(C3CCNCC3)c2O1 ; COC(=O)c1cc(OC(F)F)c2nc(CCl)n(C)c2c1 ; COC(=O)c1cc(OC(F)F)c2nc(CN3CCC(c4cccc5c4O[C@H](c4ccc(Cl)cc4F)C=C5)CC3)n(C)c2c1 ; Cn1c(CN2CCC(c3cccc4c3O[C@H](c3ccc(Cl)cc3F)C=C4)CC2)nc2c(OC(F)F)cc(C(=O)O)cc21]

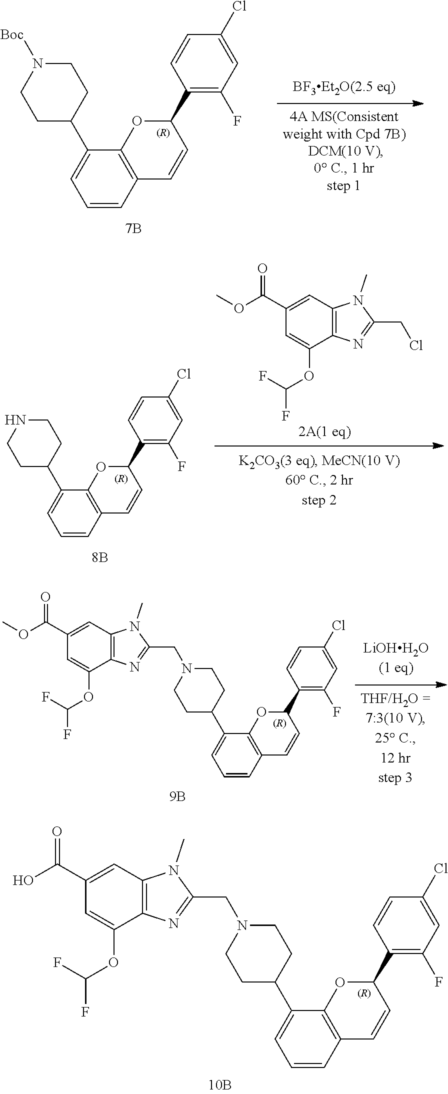

### General Procedure for Preparation of Intermediate 2:

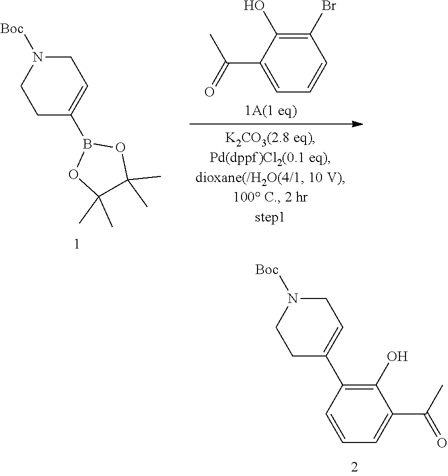 [SMILES: CC(C)(C)OC(=O)N1CC=C(B2OC(C)(C)C(C)(C)O2)CC1 ; CC(=O)c1cccc(Br)c1O ; CC(=O)c1cccc(C2=CCN(C(=O)OC(C)(C)C)CC2)c1O]

To a solution of 1A (8 g, 37.20 mmol, 1 eq) and 1 (12.64 g, 40.92 mmol, 1.1 eq) in dioxane (80 mL) and H₂O (20 mL) was added K₂CO₃ (14.40 g, 104.16 mmol, 2.8 eq) and Pd(dppf)Cl₂ (2.72 g, 3.72 mmol, 0.1 eq). Then the mixture was degassed and purged with N₂ for three times. Then the mixture was stirred at 100° C. for 2 hr. LC-MS showed 1 was consumed completely and one main peak with desired mass was detected. TLC (Petroleum ether:Ethyl acetate=5:1, R_{f}=0.5) showed the reaction was completed. The reactions mixture was filtered and concentrated in vacuum. The residue was purified by silica gel chromatography (Ethyl acetate/Petroleum ether gradient=0/1 to 30/1). 2 (9.5 g, 29.93 mmol, 80.46% yield) as a yellow oil.

LCMS: RT=0.580 min, MS: 317.2, [M+H-Boc]⁺=218.2

HPLC: product: RT=4.341 min, purity: 98.09%

¹H NMR (400 MHz, CHCl3-d) δ=12.69 (s, 1H), 7.60-7.63 (m, 1H), 7.60 (dd, J=8.0, 1.6 Hz, 1H), 7.29 (dd, J=7.6, 1.6 Hz, 1H), 6.80 (t, J=7.6 Hz, 1H), 5.80 (dt, J=3.2, 1.6 Hz, 1H), 4.00 (d, J=3.2 Hz, 2H), 3.48-3.58 (m, 2H), 2.58 (s, 3H), 2.40-2.49 (m, 2H), 1.42 (s, 9H)

### General Procedure for Preparation of Intermediate 3:

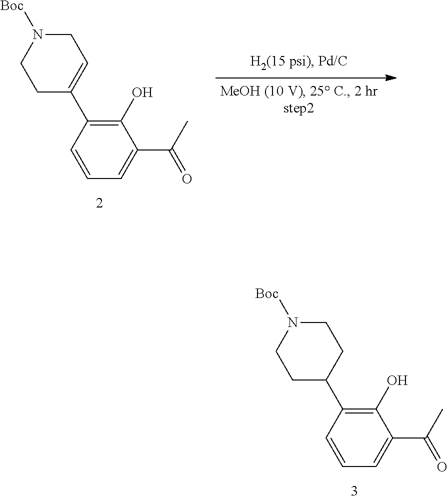 [SMILES: CC(=O)c1cccc(C2=CCN(C(=O)OC(C)(C)C)CC2)c1O ; CC(=O)c1cccc(C2CCN(C(=O)OC(C)(C)C)CC2)c1O]

To a mixture of 2 (5.9 g, 18.59 mmol, 1 eq) and Pd/C (1.98 g, 1.86 mmol, 10% purity) in MeOH (80 mL). Then the mixture was degassed and purged with H₂ (15 Psi) for three times. The mixture was stirred at 25° C. for 2 hr under H₂ (15 Psi). LCMS showed 2 was consumed completely and one main peak with desired mass was detected. The suspension was filtered, and the filter cake was washed with MeOH (50 mL*3). The mixture was filtered to obtain the organic phase. The residue was used in the next step without further purification. 3 (5.5 g, 17.22 mmol, 92.63% yield) was obtained as a brown solid.

LCMS: RT=0.592 min, MS: 319.2, [M+H−t-Bu]⁺=264.1

¹H NMR (400 MHz, CHCl3-d) δ=12.75 (s, 1H), 7.62 (d, J=8.0 Hz, 1H), 7.36 (d, J=7.6 Hz, 1H), 6.88 (t, J=7.6 Hz, 1H), 4.24 (br s, 2H), 3.24-3.11 (m, 1H), 2.85 (br s, 2H), 2.64 (s, 3H), 1.84 (br d, J=12.8 Hz, 2H), 1.55 (br d, J=12.4 Hz, 2H), 1.50-1.46 (m, 9H)

### General Procedure for Preparation of Intermediate 4:

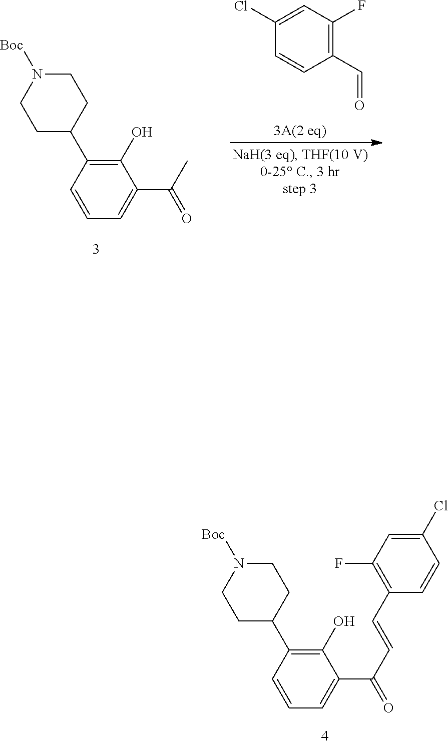 [SMILES: CC(=O)c1cccc(C2CCN(C(=O)OC(C)(C)C)CC2)c1O ; O=Cc1ccc(Cl)cc1F ; CC(C)(C)OC(=O)N1CCC(c2cccc(C(=O)/C=C/c3ccc(Cl)cc3F)c2O)CC1]

To a mixture of 3 (5.2 g, 16.28 mmol, 1 eq) in THF (52 mL) was added NaH (1.95 g, 48.84 mmol, 60% purity, 3 eq) at 0° C. under N₂. After 20 mins, the 3A (5.16 g, 32.56 mmol, 2 eq) in THF (13 mL) was added at 0° C. The mixture was stirred at 25° C. for 3 hr. LCMS (RT=0.758 min) showed 3 was consumed completely and one main peak with desired mass was detected. The reaction mixture was quenched by saturated NH₄Cl 80 mL, then extracted with EtOAc 120 mL (40 mL*3). The combined organic layers were washed with brine 80 mL, dried over Na₂SO₄, filtered, and concentrated under reduced pressure to give a residue. The residue was used to next step without further purification. 4 (3.7 g, 8.04 mmol, 49.41% yield) was obtained as a yellow solid.

LCMS: RT=2.735 min, MS cal: 459.2, [M+H−Boc]⁺=360.2

HPLC: product: RT=3.870 min, purity: 87.90%

¹H NMR (400 MHz, DMSO-d6) δ=13.26 (s, 1H), 8.28-8.15 (m, 3H), 7.91 (d, J=15.6 Hz, 1H), 7.61 (dd, J=2.0, 10.4 Hz, 1H), 7.55 (d, J=6.8 Hz, 1H), 7.45 (dd, J=2.0, 8.4 Hz, 1H), 7.00 (t, J=7.6 Hz, 1H), 4.16-4.04 (m, 2H), 3.19-3.06 (m, 1H), 2.95-2.74 (m, 2H), 1.75 (br d, J=12.4 Hz, 2H), 1.51 (dq, J=4.0, 12.4 Hz, 2H), 1.42 (s, 9H)

### General Procedure for Preparation of Intermediate 5:

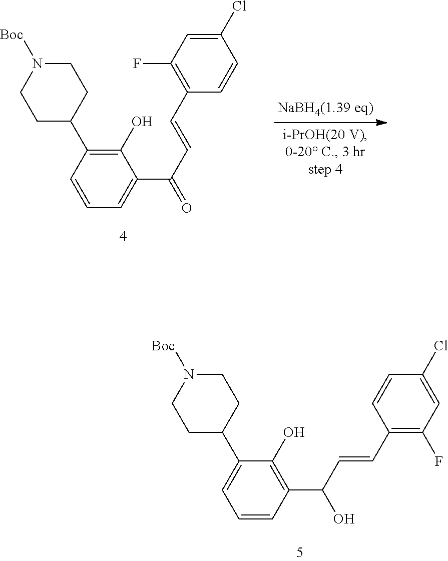 [SMILES: CC(C)(C)OC(=O)N1CCC(c2cccc(C(=O)/C=C/c3ccc(Cl)cc3F)c2O)CC1 ; CC(C)(C)OC(=O)N1CCC(c2cccc(C(O)/C=C/c3ccc(Cl)cc3F)c2O)CC1]

To a mixture of 4 (1.4 g, 3.04 mmol, 1 eq) in i-PrOH (28 mL) was added NaBH₄ (0.16 g, 4.23 mmol, 1.39 eq) at 0° C. under N₂. The mixture was stirred at 20° C. for 3 hr. The color of the mixture turned to yellow. LCMS showed 4 was consumed completely and one main peak with desired mass was detected. TLC (Petroleum ether:Ethyl acetate=10:1, product R_{f}=0.3) indicated starting material remained, and one major new spot was detected. The reaction mixture was quenched by H₂O 200 mL, then extracted with EtOAc 120 mL (40 mL*3). The combined organic layers were washed with brine 100 mL, dried over Na₂SO₄, filtered, and concentrated under reduced pressure to give a residue. The residue was purified by column chromatography (SiO₂, Petroleum ether/Ethyl acetate=100/1 to 100/10). 5 (0.6 g, 1.30 mmol, 42.67% yield) was obtained as a yellow solid.

LCMS: RT=0.655 min, MS cal.: 461.1, [M+Na]⁺=484.1.

### General Procedure for Preparation of Intermediate 6:

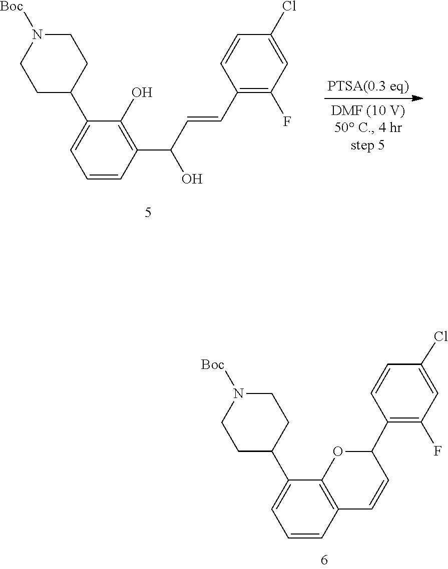 [SMILES: CC(C)(C)OC(=O)N1CCC(c2cccc(C(O)/C=C/c3ccc(Cl)cc3F)c2O)CC1 ; CC(C)(C)OC(=O)N1CCC(c2cccc3c2OC(c2ccc(Cl)cc2F)C=C3)CC1]

To a mixture of 5 (800.00 mg, 1.73 mmol, 1 eq) in DMF (8 mL) was added PTSA (89.46 mg, 519.53 μmol, 0.3 eq). The mixture was stirred at 50° C. for 4 hr. The color of the mixture turned yellow. LCMS showed the product was detached. TLC (Petroleum ether:Ethyl acetate=5:1, R_{f}=0.5) showed 4 was consumed completely and one main peak with desired mass was detected. The crude product was purified by prep-TLC (Petroleum ether:Ethyl acetate=5:1, R_{f}=0.4). 6 (650 mg, 14.64 mmol, 85.30% yield) was obtained as a white solid.

LCMS: RT=1.005 min, MS cal.: 443.2, [M−Me+H]⁺=429.2

### General Procedure for Preparation of Intermediate 7A & 7B:

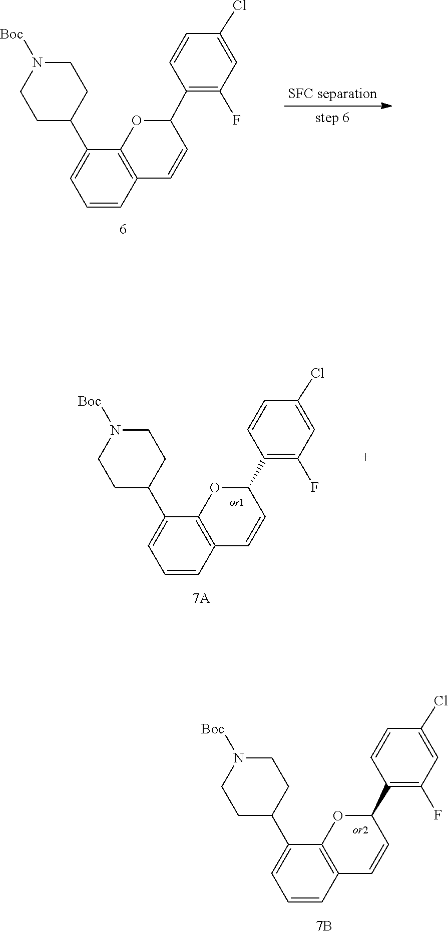 [SMILES: CC(C)(C)OC(=O)N1CCC(c2cccc3c2OC(c2ccc(Cl)cc2F)C=C3)CC1 ; CC(C)(C)OC(=O)N1CCC(c2cccc3c2O[C@H](c2ccc(Cl)cc2F)C=C3)CC1 ; CC(C)(C)OC(=O)N1CCC(c2cccc3c2O[C@@H](c2ccc(Cl)cc2F)C=C3)CC1]

The residue was separated by SFC (column: DAICEL CHIRALCEL OD (250 mm*30 mm, 10 um); mobile phase: [CO₂-MeOH (0.1% NH₃H₂O)]; B %: 20%, isocratic elution mode). 7A (100 mg, 225.26 μmol, 13.01% yield) and 7B (76 mg, 171.20 μmol, 17.19% yield) was obtained as a yellow solid.

LCMS 7A: RT=1.042 min, MS cal.: 443.1, [M−t-Bu]⁺=388.2

LCMS 7B: RT=2.772 min, MS cal.: 443.1, [M−t-Bu]⁺=388.1

¹H NMR7A (400 MHz, MeOH-d4) δ=7.40-7.46 (m, 1H), 7.28 (dd, J=10.0, 2.0 Hz, 1H), 7.19 (dd, J=8.4, 1.6 Hz, 1H), 7.03 (dd, J=7.6, 1.6 Hz, 1H), 6.93-6.97 (m, 1H), 6.84-6.89 (m, 1H), 6.71 (dd, J=10.0, 1.6 Hz, 1H), 6.23 (dd, J=3.6, 1.6 Hz, 1H), 5.85 (dd, J=10.0, 4.0 Hz, 1H), 4.08-4.21 (m, 2H), 2.93-3.04 (m, 1H), 2.70-2.88 (m, 2H), 1.73 (br dd, J=12.8, 2.0 Hz, 1H), 1.50-1.64 (m, 1H), 1.46-1.48 (m, 2H), 1.47 (s, 9H)

¹H NMR7B (400 MHz, MeOH-d4) δ=7.52 (dd, J=10.4, 1.6 Hz, 1H) 7.34-7.41 (m, 1H) 7.27-7.32 (m, 1H) 7.26-7.27 (m, 1H) 7.03 (d, J=7.2 Hz, 1H) 6.99 (d, J=7.6 Hz, 1H) 6.82-6.88 (m, 1H) 6.74 (d, J=10.0 Hz, 1H) 6.24 (d, J=3.6 Hz, 1H) 5.91 (dd, J=10.0, 4.0 Hz, 1H) 3.93-4.06 (m, 2H) 2.83-2.95 (m, 1H) 2.67-2.79 (m, 2H) 1.56-1.67 (m, 1H) 1.35-1.43 (m, 12H)

### General Procedure for Preparation of Intermediate 8A:

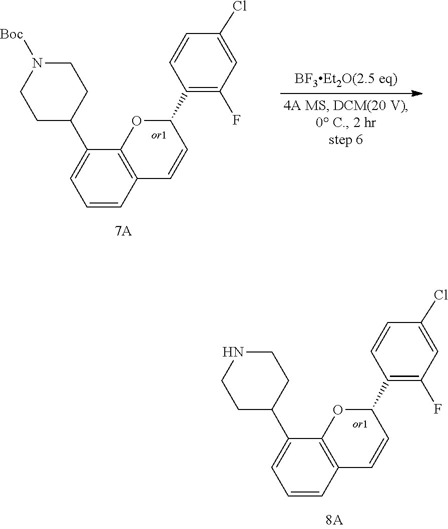 [SMILES: CC(C)(C)OC(=O)N1CCC(c2cccc3c2O[C@H](c2ccc(Cl)cc2F)C=C3)CC1 ; Fc1cc(Cl)ccc1[C@@H]1C=Cc2cccc(C3CCNCC3)c2O1]

7A (120 mg, 270.31 μmol, 1 eq) and DCM (0.8 mL) were added to a 10 mL flask, and 4A MOLECULAR SIEVE (40 mg) and BF₃·Et₂O (204.07 mg, 675.77 μmol, 176.83 μL, 47% purity, 2.5 eq) were added at 0° C. The reaction solution was reacted at 0° C. for 2 h. LCMS (RT=0.436 min) showed 7A was consumed completely and one main peak with desired mass was detected. The reaction mixture was quenched by addition H₂O 5 mL at 0° C. and extracted with EtOAc 6 mL (2 mL*3) and washed with brine 5 mL. The combined organic layers were dried over anhydrous Na₂SO₄, filtered, and concentrated under reduced pressure to give a residue. The residue was used directly in the next step. 7A (76 mg, crude) was obtained as a red oil.

LCMS: RT=0.436 min, MS cal.: 343.1, [M−t-Bu]⁺=344.1

HPLC: product: RT=2.554 min, purity: 72.90%

¹H NMR (400 MHz, MeOH-d4) δ=7.28-7.34 (m, 1H), 7.16 (dd, J=10.0, 2.0 Hz, 1H), 7.06 (dd, J=8.4, 0.16 Hz, 1H), 6.95 (dd, J=7.6, 1.6 Hz, 1H), 6.80-6.84 (m, 1H), 6.73-6.78 (m, 1H), 6.59 (dd, J=10.0, 1.6 Hz, 1H), 6.11 (d, J=4.0 Hz, 1H), 5.73 (dd, J=9.6, 4.0 Hz, 1H), 2.82-3.05 (m, 3H), 2.49-2.64 (m, 2H), 1.69-1.82 (m, 1H), 1.62-1.69 (m, 1H), 1.53 (td, J=12.4, 3.6 Hz, 2H)

### General Procedure for Preparation of Intermediate 9A:

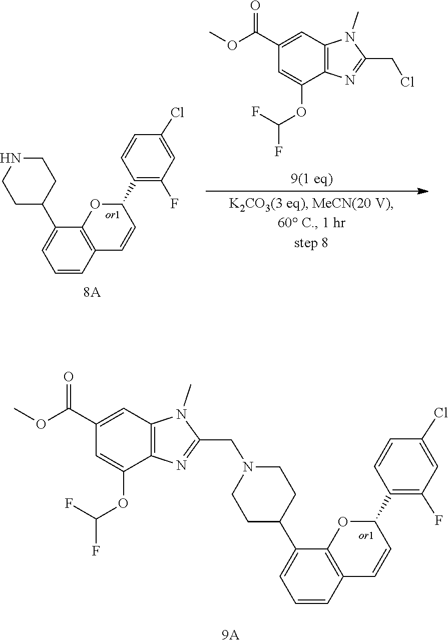 [SMILES: Fc1cc(Cl)ccc1[C@@H]1C=Cc2cccc(C3CCNCC3)c2O1 ; COC(=O)c1cc(OC(F)F)c2nc(CCl)n(C)c2c1 ; COC(=O)c1cc(OC(F)F)c2nc(CN3CCC(c4cccc5c4O[C@H](c4ccc(Cl)cc4F)C=C5)CC3)n(C)c2c1]

To a mixture of 8A (76 mg, 221.04 μmol, 1 eq) and 9 (67.35 mg, 221.04 μmol, 1 eq) in MeCN (2 mL) was added K₂CO₃ (91.65 mg, 663.13 μmol, 3 eq). The mixture was stirred at 60° C. for 2 hours. No obvious phenomenon was observed. LCMS showed 8A was consumed completely and one main peak with desired mass was detected. The reaction mixture was filtered. The residue was purified by prep-TLC (Ethyl acetate/Petroleum ether gradient=2/1). 9A (65 mg, 106.20 μmol, 48.05% yield) was obtained as a yellow oil.

LCMS: RT=0.510 min, MS cal.: 611.1, [M+H]⁺=612.3

### General Procedure for Preparation of Compound 176 (10A):

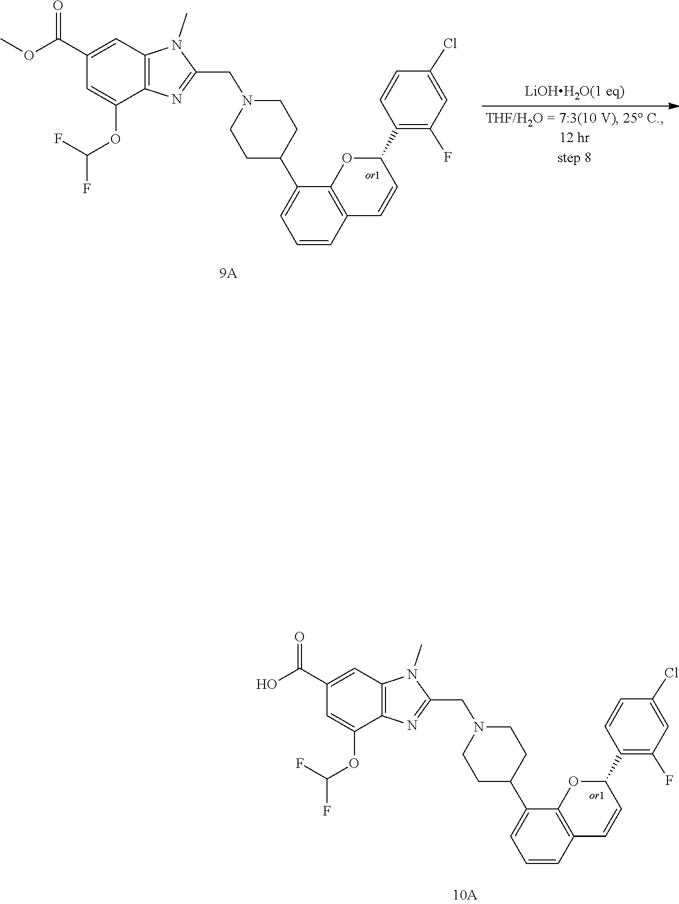 [SMILES: COC(=O)c1cc(OC(F)F)c2nc(CN3CCC(c4cccc5c4O[C@H](c4ccc(Cl)cc4F)C=C5)CC3)n(C)c2c1 ; Cn1c(CN2CCC(c3cccc4c3O[C@H](c3ccc(Cl)cc3F)C=C4)CC2)nc2c(OC(F)F)cc(C(=O)O)cc21]

To a solution of 9A (65 mg, 106.20 μmol, 1 eq) and lithium hydroxide hydrate (5.79 mg, 138.06 μmol, 1.3 eq) in THF (0.5 mL) and H₂O (0.2 mL). Then the mixture was stirred at 25° C. for 18 hr. No obvious phenomenon was observed. LCMS (ET83293-49-P1A6,) showed 9A was consumed completely and one main peak with desired mass was detected. The reaction mixture was concentrated under reduced pressure to give a residue. The residue was purified by prep-HPLC (column: Waters Xbridge Prep OBD C₁₈ 150*40 mm*10 um; mobile phase: [H₂O (10 mM NH₄HCO₃)-ACN]; gradient: 35%-65% B over 8.0 min). (S)-2-((4-(2-(4-Chloro-2-fluorophenyl)-2H-chromen-8-yl)piperidin-1-yl)methyl)-4-(difluoromethoxy)-1-methyl-1H-benzo[d]imidazole-6-carboxylic acid (26.01 mg, 43.49 μmol, 40.95% yield) as a white solid.

LCMS: RT=1.766 min, MS: 597.1, [M+H]⁺=598.1

HPLC: RT=12.469 min, purity: 100%

¹H NMR (400 MHz, MeOH-d4) δ=8.07-8.11 (m, 1H), 7.68 (s, 1H), 7.40-7.44 (m, 1H), 7.26 (m, 1H), 7.23 (t, J=74 Hz, 1H), 7.16 (d, J=8.4 Hz, 1H), 6.90-6.94 (m, 1H), 6.81-6.87 (m, 1H), 6.66-6.70 (m, 1H), 6.21 (d, J=3.67 Hz, 1H), 5.83 (dd, J=9.6, 4.0 Hz, 1H), 3.98 (s, 3H), 3.93 (s, 2H), 2.96-3.07 (m, 2H), 2.81-2.91 (m, 1H), 2.27-2.38 (m, 2H), 1.72-1.80 (m, 2H), 1.62-1.71 (m, 1H), 1.54 (br d, J=1.2 Hz, 1H)

### General Procedure for Preparation of Intermediate 8B:

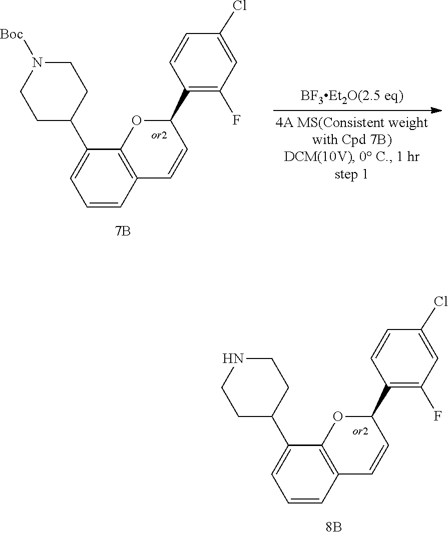 [SMILES: CC(C)(C)OC(=O)N1CCC(c2cccc3c2O[C@@H](c2ccc(Cl)cc2F)C=C3)CC1 ; Fc1cc(Cl)ccc1[C@H]1C=Cc2cccc(C3CCNCC3)c2O1]

To a mixture of 7B (91 mg, 204.98 μmol, 1 eq) and 4A MOLECULAR SIEVE (91 mg) in DCM (1 mL) was added BF₃·Et₂O (181.83 mg, 512.46 μmol, 157.57 μL, 40% purity, 2.5 eq) at 0° C. under N₂. The mixture was degassed and purged with N₂ for 3 times. The mixture was stirred at 0° C. for 1 hour. LC-MS (product: RT=0.442 min) showed 7B was consumed completely and one main peak with desired mass was detected. The reaction mixture was quenched by saturated NaHCO₃ solution to pH=8. The mixture filtered. The aqueous phase was extracted with DCM (10 mL*3). The combined organic phase was washed with brine (10 mL*3), dried with anhydrous Na₂SO₄, filtered, and concentrated in vacuum. 2 (63 mg, crude) was obtained as yellow solid.

LCMS: RT=0.442 min, MS cal.: 343.1, [M+H]⁺=344.2

¹H NMR (400 MHz, MeOH-d4) δ=7.41 (t, J=8.0 Hz, 1H), 7.26 (dd, J=10.0, 2.0 Hz, 1H), 7.17 (dd, J=8.4, 1.6 Hz, 1H), 7.05 (dd, J=7.6, 1.2 Hz, 1H), 6.92-6.96 (m, 1H), 6.83-6.90 (m, 1H), 6.69 (dd, J=10.0, 1.6 Hz, 1H), 6.20-6.25 (m, 1H), 5.84 (dd, J=10.0, 4.0 Hz, 1H), 3.17 (br d, J=12.4 Hz, 1H), 3.08-3.14 (m, 1H), 2.94-3.06 (m, 1H), 2.76-2.82 (m, 1H), 2.67-2.75 (m, 1H), 1.77-1.85 (m, 1H), 1.64-1.74 (m, 1H), 1.48-1.60 (m, 2H)

### General Procedure for Preparation of Intermediate 9B:

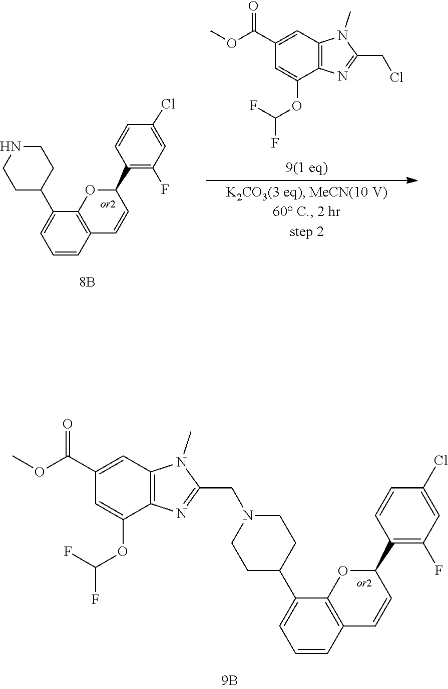 [SMILES: Fc1cc(Cl)ccc1[C@H]1C=Cc2cccc(C3CCNCC3)c2O1 ; COC(=O)c1cc(OC(F)F)c2nc(CCl)n(C)c2c1 ; COC(=O)c1cc(OC(F)F)c2nc(CN3CCC(c4cccc5c4O[C@@H](c4ccc(Cl)cc4F)C=C5)CC3)n(C)c2c1]

To a mixture of 8B (63 mg, 183.23 μmol, 1 eq) and 9 (55.83 mg, 183.23 μmol, 1 eq) in ACN (1 mL) was added K₂CO₃ (75.97 mg, 549.70 μmol, 3 eq). The mixture was stirred at 60° C. for 2 hr. LC-MS (product: RT=0.519 min) showed 8B was consumed completely and one main peak with desired mass was detected. The mixture filtered and concentrated in vacuum. The residue was purified by prep-TLC (SiO₂, PE:EtOAc=2:1). 9B (70 mg, 114.37 μmol, 62.42% yield) was obtained as a white solid.

LCMS: RT=0.525 min, MS cal.: 611.2, [M+H]⁺=612.4

### General Procedure for Preparation of Compound 175 (10B):

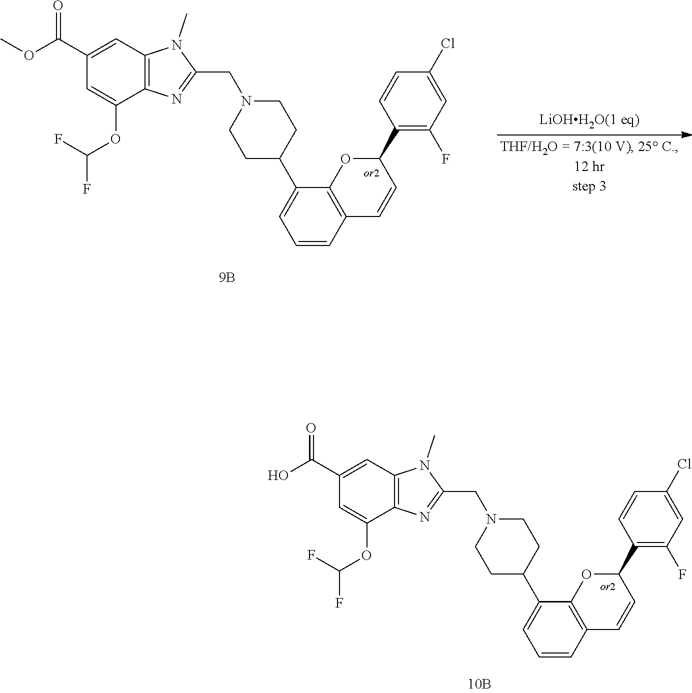 [SMILES: COC(=O)c1cc(OC(F)F)c2nc(CN3CCC(c4cccc5c4O[C@@H](c4ccc(Cl)cc4F)C=C5)CC3)n(C)c2c1 ; Cn1c(CN2CCC(c3cccc4c3O[C@@H](c3ccc(Cl)cc3F)C=C4)CC2)nc2c(OC(F)F)cc(C(=O)O)cc21]

To a mixture of 9B (70.00 mg, 114.37 μmol, 1 eq) in H₂O (0.6 mL) and THF (1.4 mL) was added LiOH·H₂O (4.80 mg, 114.37 μmol, 1 eq). The mixture was stirred at 25° C. for 12 hours. LC-MS (product: RT=1.771 min) showed 9B was consumed completely and one main peak with desired mass was detected. The residue was purified by prep-HPLC (nature condition: Waters Xbridge Prep OBD C18 150*40 mm*10 um; mobile phase: [H₂O (10 mM NH₄HCO₃)-ACN]; gradient: 20%-70% B over 8.0 min). (R)-2-((4-(2-(4-Chloro-2-fluorophenyl)-2H-chromen-8-yl)piperidin-1-yl)methyl)-4-(difluoromethoxy)-1-methyl-1H-benzo[d]imidazole-6-carboxylic acid (28.94 mg, 48.33 μmol, 42.26% yield, 99.87% purity) was obtained as a white solid.

LCMS: RT=2.863 min, MS cal.: 597.2, [M+H]⁺=598.2

HPLC: RT=12.477 min, purity: 99.87%

¹H NMR (400 MHz, DMSO-d₆) δ=8.05 (d, J=1.6 Hz, 1H), 7.62 (t, J=74.8 Hz, 1H), 7.49-7.51 (m, 2H), 7.32-7.38 (m, 1H), 7.25-7.30 (m, 1H), 6.95-6.99 (m, 1H), 6.80-6.87 (m, 1H), 6.74 (dd, J=9.6, 1.2 Hz, 1H), 6.21-6.25 (m, 1H), 5.91 (dd, J=9.6, 4.0 Hz, 1H), 3.92 (s, 3H), 3.81 (s, 2H), 2.81-2.95 (m, 2H), 2.68-2.78 (m, 1H), 2.06-2.21 (m, 2H), 1.56-1.69 (m, 2H), 1.42-1.55 (m, 1H), 1.37 (br d, J=11.6 Hz, 1H)

### Example A176—Preparation of Compound 177. 4-(Difluoromethoxy)-2-((4-(6-((2-fluoro-4-(tetrahydrofuran-2-yl)benzyl)oxy)pyridin-2-yl)piperidin-1-yl)methyl)-1-methyl-1H-benzo[d]imidazole-6-carboxylic acid (Compound 177) (5)

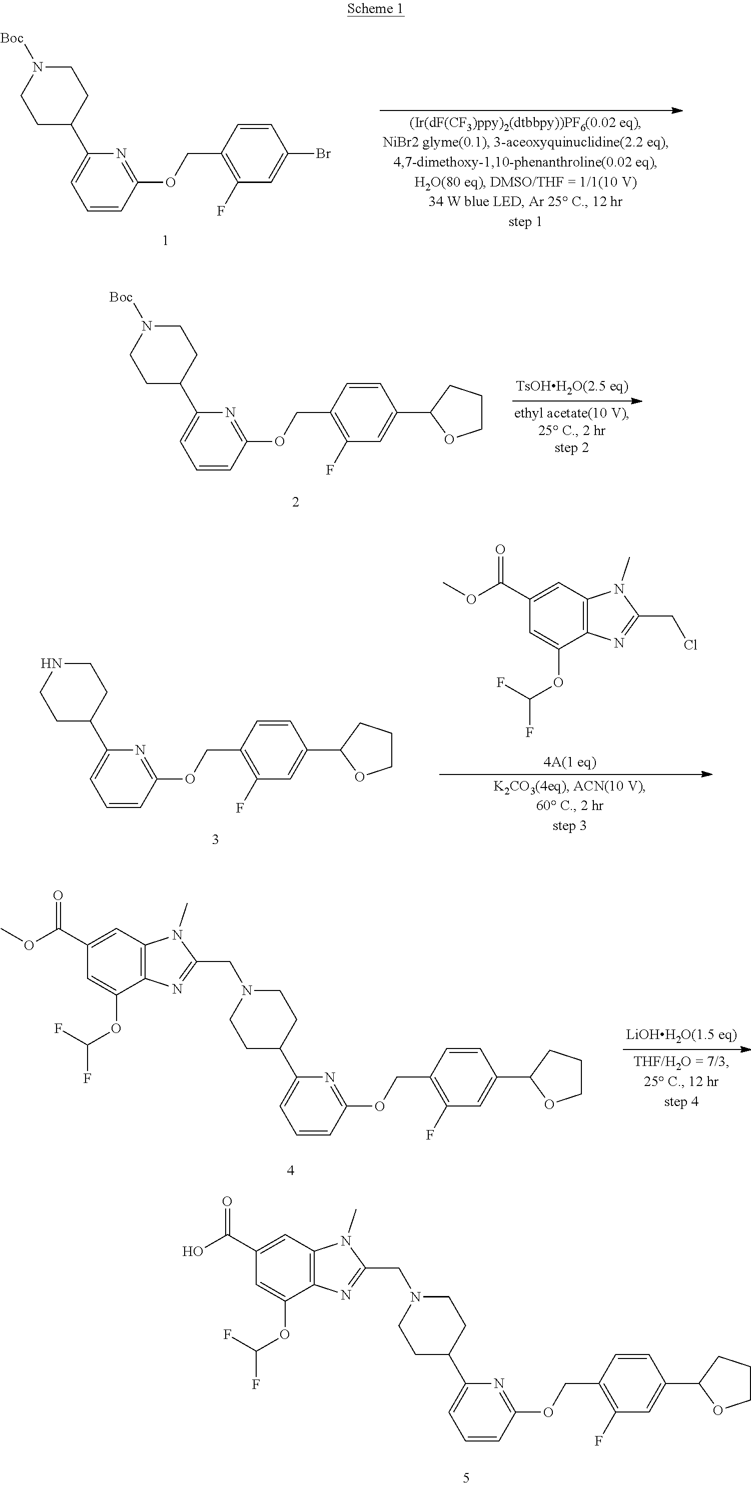 [SMILES: CC(C)(C)OC(=O)N1CCC(c2cccc(OCc3ccc(Br)cc3F)n2)CC1 ; CC(C)(C)OC(=O)N1CCC(c2cccc(OCc3ccc(C4CCCO4)cc3F)n2)CC1 ; Fc1cc(C2CCCO2)ccc1COc1cccc(C2CCNCC2)n1 ; COC(=O)c1cc(OC(F)F)c2nc(CCl)n(C)c2c1 ; COC(=O)c1cc(OC(F)F)c2nc(CN3CCC(c4cccc(OCc5ccc(C6CCCO6)cc5F)n4)CC3)n(C)c2c1 ; Cn1c(CN2CCC(c3cccc(OCc4ccc(C5CCCO5)cc4F)n3)CC2)nc2c(OC(F)F)cc(C(=O)O)cc21]

### General Procedure for Preparation of Intermediate 2:

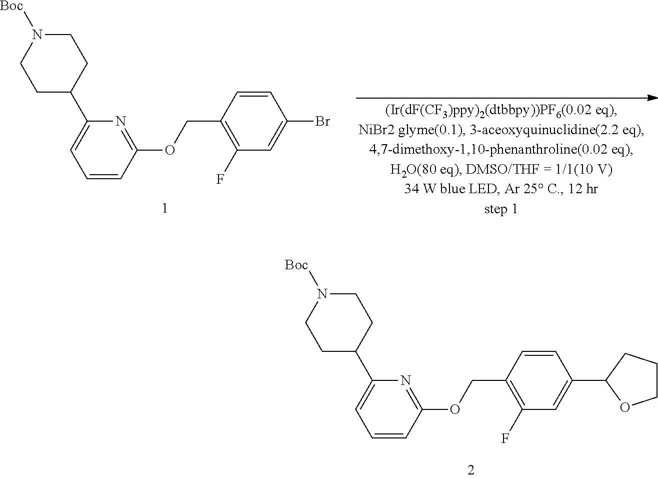 [SMILES: CC(C)(C)OC(=O)N1CCC(c2cccc(OCc3ccc(Br)cc3F)n2)CC1 ; CC(C)(C)OC(=O)N1CCC(c2cccc(OCc3ccc(C4CCCO4)cc3F)n2)CC1]

To a solution of 1 (100 mg, 214.89 μmol, 1 eq) in DMSO (0.5 mL) and THF (0.5 mL) was added (Ir(dF(CF₃)ppy)₂(dtbbpy))PF₆ (4.82 mg, 4.30 μmol, 0.02 eq), NiBr₂ glyme (6.63 mg, 21.49 μmol, 0.1 eq), 3-aceoxyquinuclidine (80.00 mg, 472.76 μmol, 2.2 eq), 4,7-dimethoxy-1,10-phenanthroline (1.03 mg, 4.30 μmol, 0.02 eq) and H₂O (309.79 mg, 17.19 mmol, 309.79 μL, 80 eq). The mixture was stirred at 50° C. for 12 hr at 34W blue LED under Ar atmosphere. The reaction mixture was quenched by addition H₂O 10 mL at 20° C. and extracted with ethyl acetate 9 mL (3 mL*3). The combined organic layers were washed with brine 10 mL (5 mL*2), dried over Na₂SO₄, filtered, and concentrated under reduced pressure to give a residue. The residue was purified by column chromatography (SiO₂, Petroleum ether/Ethyl acetate=10/1 to 1/1). 2 (200 mg, 438.07 μmol, 50.96% yield) was obtained as a colorless oil.

¹H NMR (400 MHz, DMSO-d6) δ=7.62 (t, J=7.6 Hz, 1H), 7.47 (t, J=7.6 Hz, 1H), 7.14 (s, 1H), 7.11 (s, 1H), 6.86 (d, J=7.2 Hz, 1H), 6.66 (d, J=8.0 Hz, 1H), 5.35 (s, 2H), 4.82 (t, J=7.2 Hz, 1H), 4.11-4.01 (m, 2H), 4.01-3.93 (m, 1H), 3.80 (q, J=7.2 Hz, 1H), 2.81-2.71 (m, 2H), 2.35-2.24 (m, 1H), 1.91 (quin, J=7.2 Hz, 2H), 1.76 (d, J=12.0 Hz, 2H), 1.69-1.49 (m, 3H), 1.42 (s, 9H)

### General Procedure for Preparation of Intermediate 3:

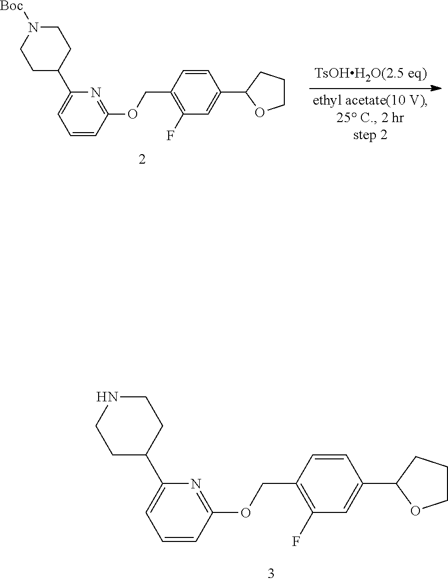 [SMILES: CC(C)(C)OC(=O)N1CCC(c2cccc(OCc3ccc(C4CCCO4)cc3F)n2)CC1 ; Fc1cc(C2CCCO2)ccc1COc1cccc(C2CCNCC2)n1]

A mixture of 2 (200 mg, 438.07 μmol, 1 eq) and TsOH·H₂O (208.32 mg, 1.10 mmol, 2.5 eq) in ethyl acetate (2 mL) was degassed and purged with N₂ for 3 times, and then the mixture was stirred at 60° C. for 2 hr under N₂ atmosphere. LCMS (RT=0.398 min) showed the 2 was consumed completely. The reaction mixture was concentrated under reduced pressure to remove solvent. The residue was diluted with DCM 3 mL, added to sat. Na₂CO₃ to adjust pH=9-10 and extracted with DCM 9 mL (3 mL*3). The combined organic layers were washed with brine 10 mL (5 mL*2), dried over Na₂SO₄, filtered, and concentrated under reduced pressure to give a residue. 3 (100 mg, crude) was obtained as a brown solid.

### General Procedure for Preparation of Intermediate 4:

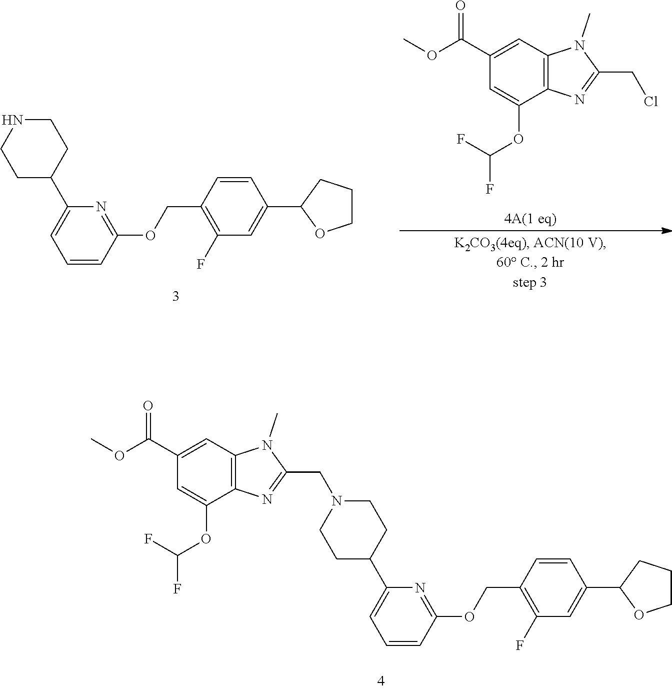 [SMILES: Fc1cc(C2CCCO2)ccc1COc1cccc(C2CCNCC2)n1 ; COC(=O)c1cc(OC(F)F)c2nc(CCl)n(C)c2c1 ; COC(=O)c1cc(OC(F)F)c2nc(CN3CCC(c4cccc(OCc5ccc(C6CCCO6)cc5F)n4)CC3)n(C)c2c1]

A mixture of 3 (100 mg, 280.56 μmol, 1 eq), K₂CO₃ (116.33 mg, 841.67 μmol, 3 eq) and 4A (59.84 mg, 196.39 μmol, 0.7 eq) in CH₃CN (1 mL) was degassed and purged with N₂ for 3 times, and then the mixture was stirred at 60° C. for 2 hr under N₂ atmosphere. LCMS (product: RT=0.337 min) showed 3 was consumed completely. The reaction mixture was concentrated under reduced pressure to remove solvent. The residue was added H₂O 3 mL and extracted with ethyl acetate 9 mL (3 mL*3). The combined organic layers were washed with brine 10 mL (5 mL*2), dried over Na₂SO₄, filtered and concentrated under reduced pressure to give a residue. The residue was purified by column chromatography (SiO₂, Petroleum ether/Ethyl acetate=1/1 to 0/1). 3 (100 mg, 160.09 μmol, 57.06% yield) was obtained as a white solid.

### General Procedure for Preparation of Compound 177 (5):

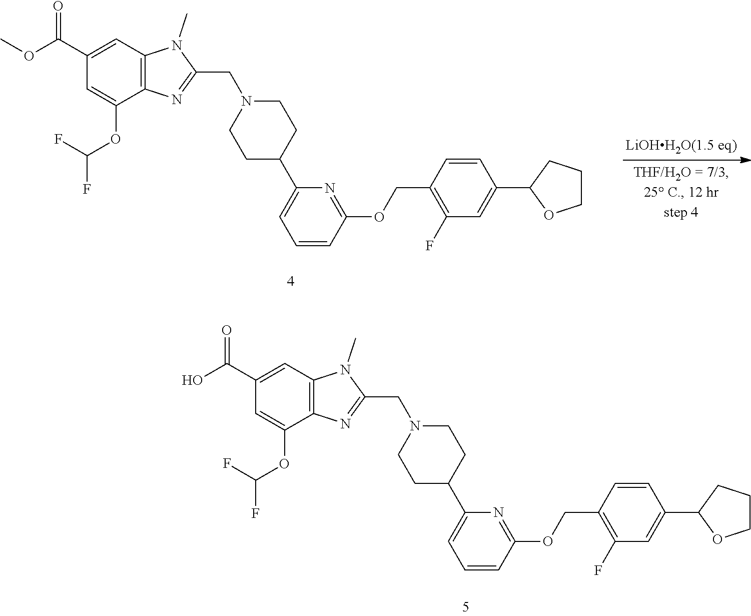 [SMILES: COC(=O)c1cc(OC(F)F)c2nc(CN3CCC(c4cccc(OCc5ccc(C6CCCO6)cc5F)n4)CC3)n(C)c2c1 ; Cn1c(CN2CCC(c3cccc(OCc4ccc(C5CCCO5)cc4F)n3)CC2)nc2c(OC(F)F)cc(C(=O)O)cc21]

To a solution of 4 (100 mg, 160.09 μmol, 1 eq) in THF (0.7 mL) was added LiOH·H₂O (10.08 mg, 240.14 μmol, 1.5 eq) in H₂O (0.3 mL). The mixture was stirred at 25° C. for 12 hr under N₂ atmosphere. LCMS (product: RT=0.458 min) showed the 4 was consumed completely. The reaction mixture was purified by prep-HPLC (column: Waters Xbridge BEH C18 100*30 mm*10 um; mobile phase: [H₂O (10 mM NH₄HCO₃)-ACN]; gradient: 15%-55% B over 8.0 min). 4-(Difluoromethoxy)-2-((4-(6-((2-fluoro-4-(tetrahydrofuran-2-yl)benzyl)oxy)pyridin-2-yl)piperidin-1-yl)methyl)-1-methyl-1H-benzo[d]imidazole-6-carboxylic acid (26.46 mg, 42.77 μmol, 26.72% yield, 98.70% purity) was obtained as a white solid.

LCMS: RT=2.701 min, MS cal.: 610.2, [M+H]⁺=611.3

HPLC: RT=11.362 min

¹H NMR (400 MHz, DMSO-d6) δ=8.08 (d, J=1.2 Hz, 1H), 7.63 (t, J=74.8 Hz, 1H), 7.60 (t, J=7.6 Hz, 1H), 7.52 (s, 1H), 7.49-7.44 (m, 1H), 7.13-7.06 (m, 2H), 6.84 (d, J=7.2 Hz, 1H), 6.64 (d, J=8.0 Hz, 1H), 5.34 (s, 2H), 4.77 (t, J=7.2 Hz, 1H), 3.97 (s, 3H), 3.95-3.89 (m, 1H), 3.85 (s, 2H), 3.79-3.72 (m, 1H), 2.93 (d, J=11.2 Hz, 2H), 2.64-2.53 (m, 1H), 2.29-2.15 (m, 3H), 1.86 (quin, J=6.8 Hz, 2H), 1.81-1.64 (m, 4H), 1.58 (qd, J=8.0, 12.0 Hz, 1H)

### Example A177—Preparation of Compound 178. 2-(((1r,4r)-4-(6-((4-Cyano-2-fluorobenzyl)oxy)pyridin-2-yl)cyclohexyl)(methyl)amino)-4-methoxy-1-methyl-1H-benzo[d]imidazole-6-carboxylic acid (Compound 178) (7)

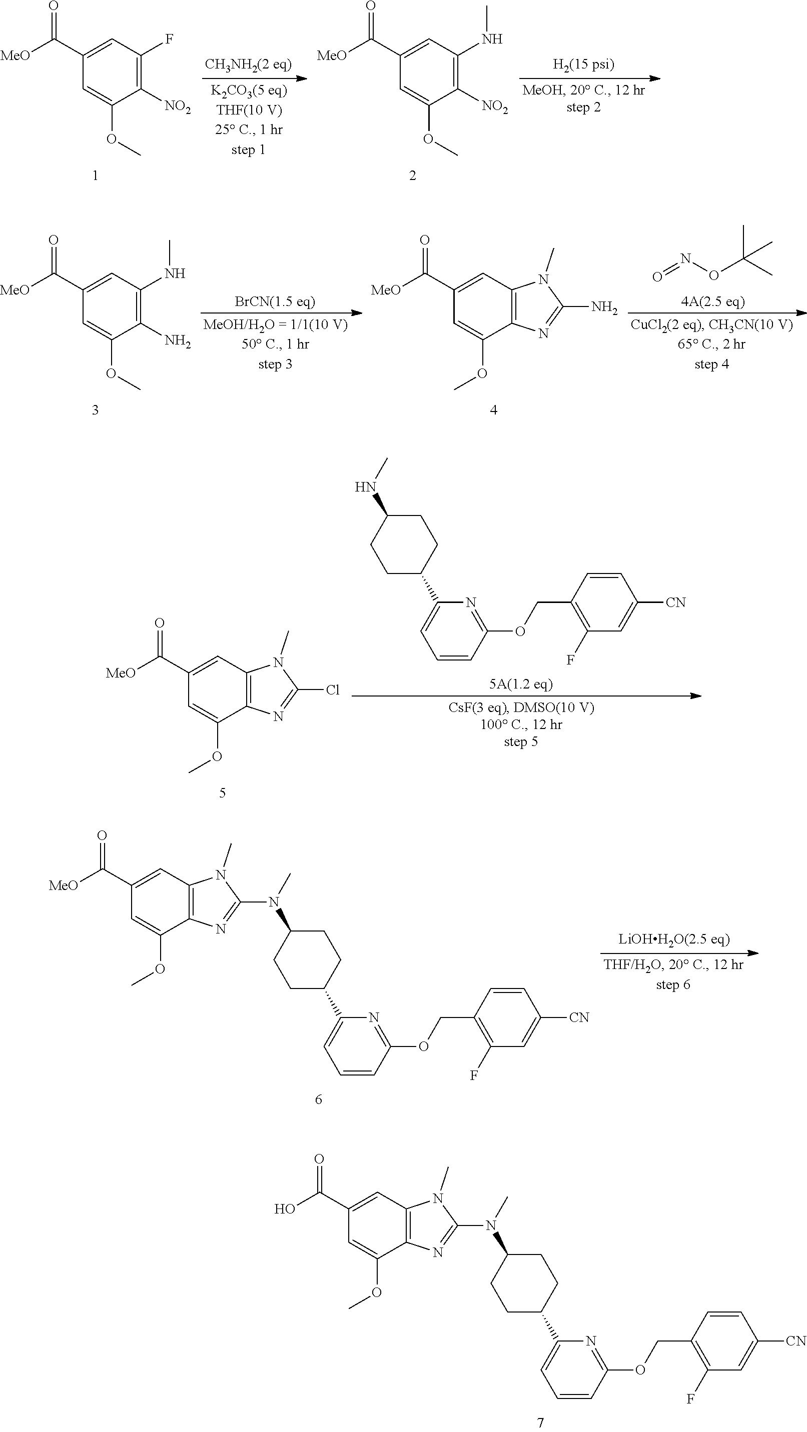

### General Procedure for Preparation of Intermediate 2:

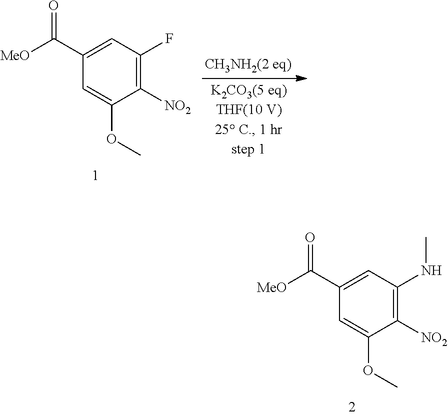 [SMILES: COC(=O)c1cc(F)c([N+](=O)[O-])c(OC)c1 ; CNc1cc(C(=O)OC)cc(OC)c1[N+](=O)[O-]]

NMP (16 mL) was charged to the three-necked round bottom flask, then 1 (1.56 g, 6.81 mmol, 1 eq) and K₂CO₃ (4.70 g, 34.04 mmol, 5 eq) was added to the mixture at 25° C. MeNH₂ (2 M, 6.81 mL, 2 eq) was added to the reaction mixture at 25° C. The mixture was stirred at 25° C. for 2 hr.

LC-MS showed 1 was consumed completely and one main peak with desired was detected. The reaction mixture was added to 20 mL H₂O at 25° C., adjusted to pH˜7 with HCl (1 M). The mixture was extracted by EtOAc (20 mL*3). Then organic phase was combined and washed by H₂O (80 mL*3), dried with Na₂SO₄, filtered. The organic phase was concentrated under reduced pressure at 45° C. to give a residue. The residue was purified by column chromatography (SiO₂, Petroleum ether/Ethyl acetate=50/1 to 10/1). 2 (1.4 g, 5.83 mmol, 85.62% yield) was obtained as an orange solid.

LCMS: RT=1.100 min, MS cal.: 240.9, [M+H]⁺=240.9

H NMR (400 MHz, CHCl3-d) δ=7.08 (d, J=1.5 Hz, 1H), 6.94 (d, J=1.5 Hz, 1H), 3.99 (d, J=9.4 Hz, 1H), 3.94 (d, J=3.8 Hz, 6H), 2.97 (s, 3H)

### General Procedure for Preparation of Intermediate 3:

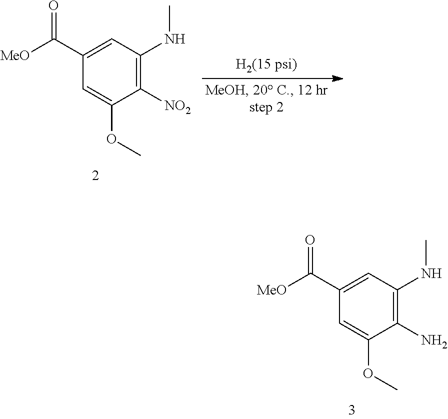 [SMILES: CNc1cc(C(=O)OC)cc(OC)c1[N+](=O)[O-] ; CNc1cc(C(=O)OC)cc(OC)c1N]

MeOH (30 mL) was charged to the one-necked round bottom flask, then Pd/C (0.6 g), MeOH (30 mL), 2 (1.4 g, 5.83 mmol, 1 eq) was added to the reaction mixture at 25° C. The mixture was stirred at 25° C. for 2 hr (15 psi). LC-MS showed 2 was consumed completely and one main peak with desired was detected. After 2 hr, the reaction mixture was filtered, washed by MeOH (50 mL*3). Then organic phase was combined and concentrated under reduced pressure at 45° C. to give a residue to give Compound 3 (1.15 g, 5.47 mmol, 93.86% yield) was obtained as a yellow solid.

LCMS: RT=0.273 min, MS cal.: 211.1, [M+H]⁺=211.1

¹H NMR (400 MHz, CHCl3-d) δ=7.19 (d, J=1.6 Hz, 1H), 7.15 (d, J=1.5 Hz, 1H), 3.90 (d, J=7.1 Hz, 6H), 2.92 (s, 3H)

### General Procedure for Preparation of Intermediate 4:

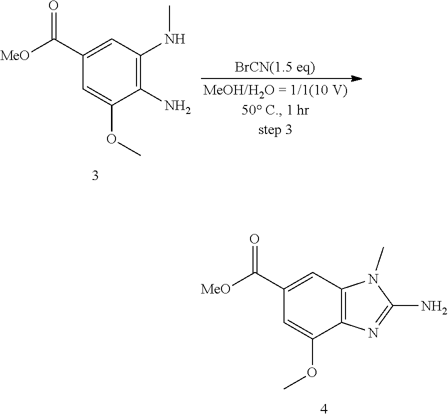 [SMILES: CNc1cc(C(=O)OC)cc(OC)c1N ; COC(=O)c1cc(OC)c2nc(N)n(C)c2c1]

MeOH (5.7 mL) and H₂O (5.7 mL) was charged to the three-necked round bottom flask, then 3 (1.15 g, 5.47 mmol, 1 eq) and BrCN (869.11 mg, 8.21 mmol, 602.29 μL, 1.5 eq) were added. The mixture was stirred at 50° C. for 2 hr. LC-MS showed 3 was consumed completely and one main peak with desired was detected. The reaction mixture was added with 1M Na₂CO₃ to adjusted pH˜8 with and then diluted with H₂O (10 mL) and extracted with EtOAc (20 mL*2). The combined organic phase was washed with brine (20 mL), dried over Na₂SO₄, filtered and concentrated under reduced pressure to give a residue. The residue was washed with EtOAc (4 mL), filtered, the solid was collected and concentrated in vacuo. 4 (0.96 g, 4.08 mmol, 74.60% yield) was obtained as a white solid.

LCMS: RT=0.273 min, MS cal.: 236.3, [M+H]⁺=236.3

¹H NMR (400 MHz, DMSO-d₆) δ=7.45 (d, J=1.0 Hz, 1H), 7.18 (d, J=1.0 Hz, 1H), 6.68 (s, 2H), 3.89 (s, 3H), 3.82 (s, 3H), 3.52 (s, 3H)

### General Procedure for Preparation of Intermediate 5:

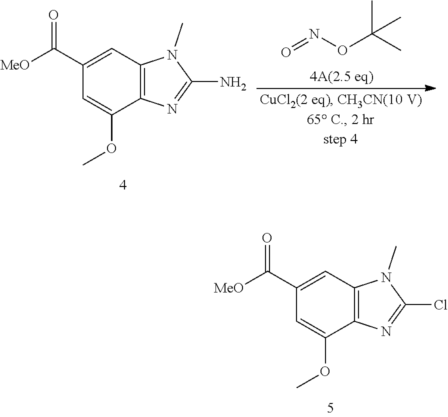

A mixture of 4 (0.2 g, 850.20 μmol, 1 eq), 4A (131.51 mg, 1.28 mmol, 151.68 μL, 1.5 eq), CuCl₂ (114.31 mg, 850.20 μmol, 1 eq), CH₃CN (2 mL) was degassed and purged with N₂ for 3 times, and then the mixture was stirred at 65° C. for 2 hrs under N₂ atmosphere. LC-MS showed 4 was consumed completely and one main peak with desired was detected. The mixture was filtered, the organic layer was concentrated in vacuo. H₂O (20 mL) was added at 25° C., extracted with EtOAc (20 mL*2). The combined organic layers were dried over Na₂SO₄, filtered, and concentrated under reduced pressure to give a residue, which was purified by column chromatography (SiO₂, Petroleum ether/Ethyl acetate=20/1 to 5/1). 5 (0.1 g, 392.67 μmol, 46.19% yield) was obtained as a light-yellow oil.

LCMS: RT=0.387 min, MS cal.: 255.0, [M+H]⁺=255.0

¹H NMR (400 MHz, CHCl3-d) δ=7.72 (d, J=1.2 Hz, 1H), 7.27 (m, 1H), 4.07 (s, 3H), 3.97 (s, 3H), 3.83 (s, 3H)

### General Procedure for Preparation of Intermediate 6:

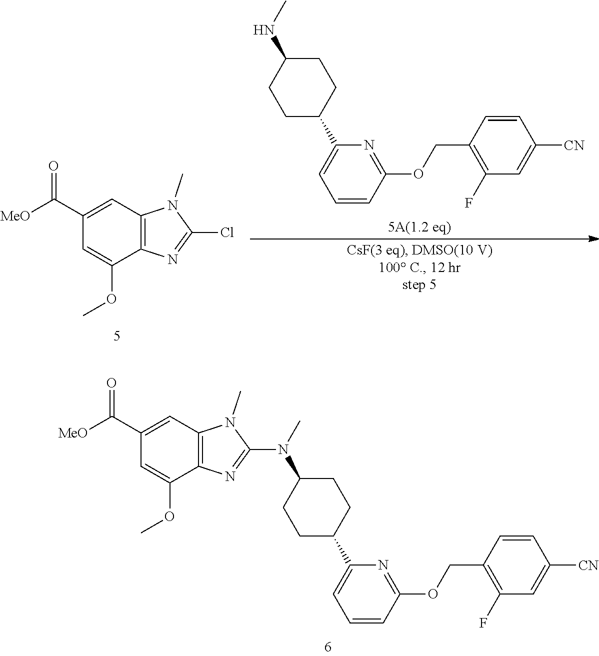 [SMILES: COC(=O)c1cc(OC)c2nc(Cl)n(C)c2c1 ; CN[C@H]1CC[C@H](c2cccc(OCc3ccc(C#N)cc3F)n2)CC1 ; COC(=O)c1cc(OC)c2nc(N(C)[C@H]3CC[C@H](c4cccc(OCc5ccc(C#N)cc5F)n4)CC3)n(C)c2c1]

In glovebox, 5 (94.54 mg, 371.24 μmol, 1.4 eq), 5A (0.09 g, 265.17 μmol, 1 eq) was added a seal tube (both 5 and 5A were dissolved in toluene (0.2 mL) and the solution was conducted under azeotropic concentration to remove moisture. The same treatment was performed three times). After addition, CsF (120.84 mg, 795.51 μmol, 3 eq) and DMSO (2 mL) was added, and the mixture was stirred at 100° C. for 12 h under N₂ atmosphere. LC-MS showed 5A was consumed completely and one main peak with desired was detected. The mixture was filtered, the organic layer was concentrated in vacuo. H₂O (20 mL) was added at 25° C., extracted with EtOAc (20 mL*2). The combined organic layers were dried over Na₂SO₄, filtered, and concentrated under reduced pressure to give a residue. The residue was purified by column chromatography (SiO₂, Petroleum ether/Ethyl acetate=20/1 to 5/1). 6 (0.11 g, 197.27 μmol, 74.39% yield) was obtained as a light yellow solid.

LCMS: RT=0.941 min, MS cal.: 632.2, [M+H]⁺=633.3

¹H NMR (400 MHz, DMSO-d₆) δ=7.92 (d, J=10.4 Hz, 1H), 7.71 (d, J=3.2 Hz, 2H), 7.67-7.60 (m, 2H), 7.25 (d, J=1.2 Hz, 1H), 6.88 (d, J=7.2 Hz, 1H), 6.71 (d, J=8.0 Hz, 1H), 5.46 (s, 2H), 3.92 (s, 3H), 3.88-3.81 (m, 3H), 3.71-3.61 (m, 3H), 3.59-3.47 (m, 1H), 2.90 (s, 3H), 2.64-2.55 (m, 1H), 1.90 (d, J=11.4 Hz, 4H), 1.81-1.71 (m, 2H), 1.64-1.51 (m, 2H)

### General Procedure for Preparation of Compound 178 (7):

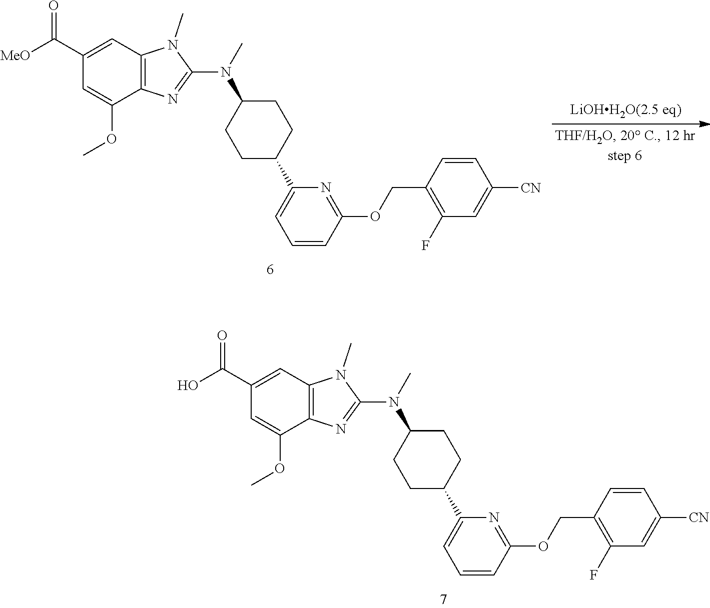 [SMILES: COC(=O)c1cc(OC)c2nc(N(C)[C@H]3CC[C@H](c4cccc(OCc5ccc(C#N)cc5F)n4)CC3)n(C)c2c1 ; COc1cc(C(=O)O)cc2c1nc(N(C)[C@H]1CC[C@H](c3cccc(OCc4ccc(C#N)cc4F)n3)CC1)n2C]

To a solution of 6 (0.09 g, 161.40 μmol, 1 eq) in THF (10 mL) and H₂O (1.2 mL) was added LiOH·H₂O (20.32 mg, 484.21 μmol, 3 eq), the mixture was stirred at 25° C. for 12 h. LC-MS showed 6 was consumed completely and one main peak with desired was detected. The mixture was adjusted to Ph˜7 with sat. NaHCO₃ aqueous, 4 mL DMSO was added in the mixture and concentrated in vacuo. The solution was purified by prep-HPLC (column: Waters Xbridge BEH C18 100*25 mm*10 um; mobile phase: [H2O(10 mM NH₄HCO₃)-ACN]; gradient: 20%-50% B over 8.0 min). 2-(((1r,4r)-4-(6-((4-Cyano-2-fluorobenzyl)oxy)pyridin-2-yl)cyclohexyl)(methyl)amino)-4-methoxy-1-methyl-1H-benzo[d]imidazole-6-carboxylic acid (0.035 g, 63.53 μmol, 39.36% yield, 98.67% purity) was obtained as a white solid.

LCMS: RT=2.689 min, MS cal.: 544.3, [M+H]⁺=544.3

HPLC: RT=11.434 min, purity: 98.67%

¹H NMR (400 MHz, MeOH-d4) δ=7.68-7.66 (m, 2H), 7.60-7.53 (m, 3H), 7.44 (s, 1H), 6.84-6.82 (d, J=8.0 Hz, 1H), 6.67-6.65 (d, J=8.0 Hz, 1H), 5.51 (s, 2H), 4.00 (s, 3H), 3.70 (s, 3H), 3.49-3.48 (m, 1H), 2.95 (s, 3H), 2.62-2.56 (m, 1H), 1.98-1.96 (m, 4H), 1.86-1.76 (m, 2H), 1.73-1.63 (m, 2H)

### Example A178—Preparation of Compound 179. 2-((4-(2-Chloro-3-((4-cyano-2-fluorobenzyl)oxy)phenyl)piperidin-1-yl)methyl)-4-(difluoromethoxy)-1-methyl-1H-benzo[d]imidazole-6-carboxylic acid (Compound 179) (7)

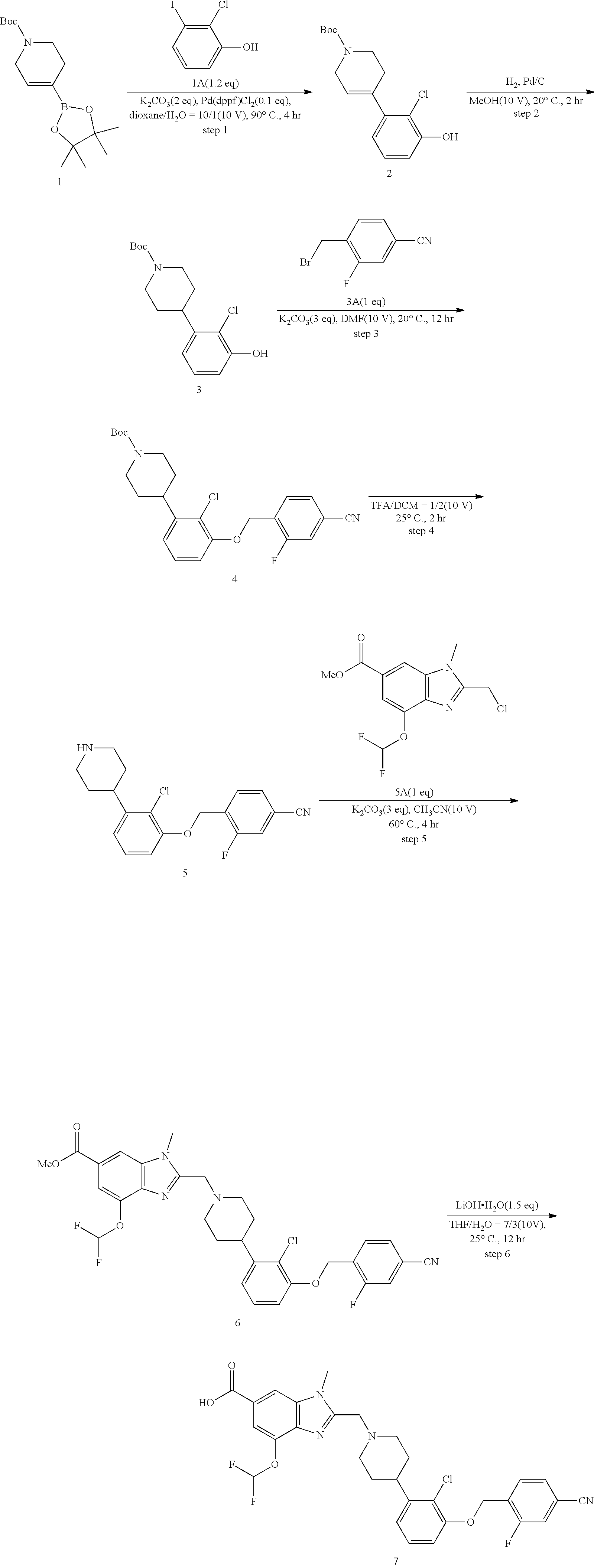

### General Procedure for Preparation of Intermediate 2:

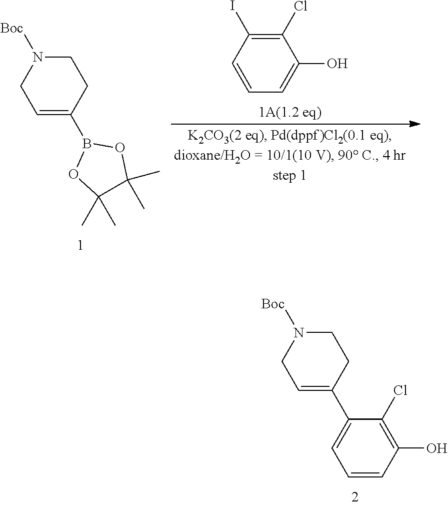 [SMILES: CC(C)(C)OC(=O)N1CC=C(B2OC(C)(C)C(C)(C)O2)CC1 ; Oc1cccc(I)c1Cl ; CC(C)(C)OC(=O)N1CC=C(c2cccc(O)c2Cl)CC1]

To a solution of 1 (550 mg, 1.78 mmol, 1 eq) in dioxane (5 mL) and H₂O (0.5 mL) was added

Pd(dppf)Cl₂ (130.15 mg, 177.87 μmol, 0.1 eq), K₂CO₃ (737.49 mg, 5.34 mmol, 3 eq) and 1A (543.13 mg, 2.13 mmol, 1.2 eq). The mixture was stirred at 90° C. for 4 hr. Several new peaks were shown on LCMS and 73% of desired 2 was detected. The reaction mixture was diluted with H₂O 5 mL and extracted with EtOAc 30 mL (10 mL*3). The combined organic layers were washed with brine 10 mL, dried over Na₂SO₄, filtered, and concentrated under reduced pressure to give a residue. The residue was purified by column chromatography (SiO₂, PE/EtOAc=1/0 to 5/1). 2 (420 mg, 1.29 mmol, 72.41% yield) was obtained as a white solid.

LCMS: RT=1.276 min, MS cal.: 309.1/311.1, [M−H]⁻=307.9/309.9

¹HNMR (400 MHz, CHCl3-d) δ=7.17-7.12 (m, 1H), 6.96 (dd, J=1.6, 8.4 Hz, 1H), 6.76 (dd, J=1.6, 7.6 Hz, 1H), 5.78-5.66 (m, 2H), 4.06 (q, J=2.8 Hz, 2H), 3.64 (t, J=5.6 Hz, 2H), 2.43 (br d, J=1.6 Hz, 2H), 1.51 (s, 9H)

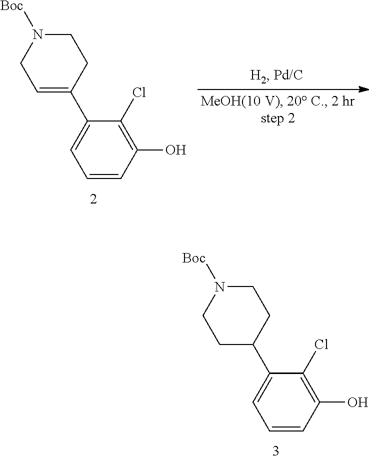 [SMILES: CC(C)(C)OC(=O)N1CC=C(c2cccc(O)c2Cl)CC1 ; CC(C)(C)OC(=O)N1CCC(c2cccc(O)c2Cl)CC1]

Equip a 50 mL one-necked round bottom flask, thermometer, H₂ balloon. MeOH (4 mL) was charged to the 10 mL one-necked round bottom flask, then 2 (400 mg, 1.29 mmol, 1 eq) was added to the mixture at 20° C. Pd/C (79.97 mg) was added dropwise to the reaction mixture at 20° C. After the addition, the mixture was stirred at 20° C. for 2 hr under H₂ (15 psi). LCMS showed 2 was consumed completely and one main peak with desired mass was detected. The reaction mixture was filtered, and the filter cake was washed with MeOH (20 mL). The combined filtrate was concentrated to dryness to give product. 3 (300 mg, 894.80 μmol, 69.30% yield) was obtained as a white solid.

LCMS: RT=1.287 min, MS cal.: 311.1/313.1, [M−H]⁻=309.9/311.9

¹HNMR (400 MHz, CHCl3-d) δ=7.19-7.13 (m, 1H), 6.92 (dd, J=1.6, 8.0 Hz, 1H), 6.81 (dd, J=1.2, 7.6 Hz, 1H), 5.78 (s, 1H), 4.27 (br d, J=8.0 Hz, 2H), 3.14-3.05 (m, 1H), 2.85 (br t, J=12.4 Hz, 2H), 1.84 (br d, J=13.2 Hz, 2H), 1.67-1.55 (m, 2H), 1.49 (s, 9H)

### General Procedure for Preparation of Intermediate 4:

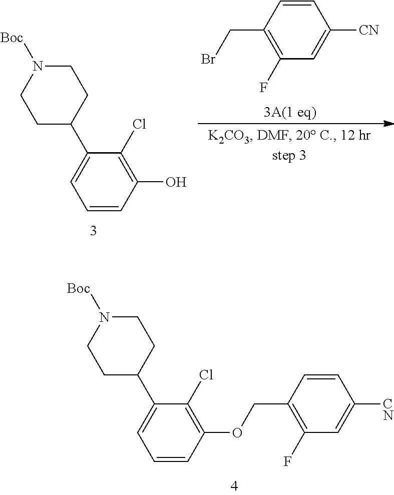 [SMILES: CC(C)(C)OC(=O)N1CCC(c2cccc(O)c2Cl)CC1 ; N#Cc1ccc(CBr)c(F)c1 ; CC(C)(C)OC(=O)N1CCC(c2cccc(OCc3ccc(C#N)cc3F)c2Cl)CC1]

To a solution of 3 (230 mg, 737.65 μmol, 1 eq) in DMF (2.5 mL) was added K₂CO₃ (305.84 mg, 2.21 mmol, 3 eq) and 3A (157.88 mg, 737.65 μmol, 1 eq). The mixture was stirred at 20° C. for 12 hr. LCMS showed 3 was consumed completely and one main peak with desired mass was detected. The reaction mixture was diluted with H₂O 5 mL and extracted with EtOAc 15 mL (5 mL*3). The combined organic layers were washed with brine 10 mL (5 mL*2), dried over Na₂SO₄, filtered, and concentrated under reduced pressure to give a residue. The residue was purified by column chromatography (SiO₂, PE/EtOAc=1/0 to 5/1). 4 (220 mg, 484.58 μmol, 65.69% yield) was obtained as a white solid.

LCMS: RT=1.620 min, MS cal.: 444.2/446.2, [M−H]⁻=442.9/444.8

¹HNMR (400 MHz, CHCl3-d) δ=7.85 (t, J=7.6 Hz, 1H), 7.54 (d, J=8.0 Hz, 1H), 7.40 (dd, J=1.2, 9.6 Hz, 1H), 7.22 (t, J=8.0 Hz, 1H), 6.93 (d, J=8.0 Hz, 1H), 6.87 (d, J=8.0 Hz, 1H), 5.25 (s, 2H), 4.31-4.24 (m, 2H), 3.25 (tt, J=3.2, 12.0 Hz, 1H), 2.87 (br t, J=12.0 Hz, 2H), 1.85 (br d, J=12.8 Hz, 2H), 1.57 (s, 2H), 1.50 (s, 9H)

### General Procedure for Preparation of Intermediate 5:

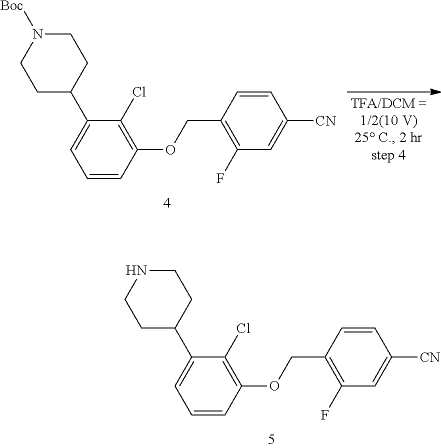 [SMILES: CC(C)(C)OC(=O)N1CCC(c2cccc(OCc3ccc(C#N)cc3F)c2Cl)CC1 ; N#Cc1ccc(COc2cccc(C3CCNCC3)c2Cl)c(F)c1]

To a solution of 4 (120 mg, 269.71 μmol, 1 eq) in DCM (0.8 mL) was added TFA (614.00 mg, 5.38 mmol, 0.4 mL, 19.97 eq). The mixture was stirred at 25° C. for 2 hr. LCMS showed 4 was consumed completely and one main peak with desired mass was detected. The reaction mixture was concentrated under reduced pressure. Without purification. 5 (120 mg, crude, TFA) was obtained as a yellow oil.

LCMS: RT=0.376 min, MS cal.: 344.1/346.1, [M+H]⁺=345.1/347.0

¹HNMR (400 MHz, CHCl3-d) δ=7.84 (t, J=7.6 Hz, 1H), 7.57-7.53 (m, 1H), 7.42 (dd, J=1.6, 9.2 Hz, 1H), 7.32-7.28 (m, 1H), 6.95 (t, J=6.8 Hz, 2H), 5.26 (s, 2H), 3.69 (br d, J=11.6 Hz, 2H), 3.48-3.39 (m, 1H), 3.19 (br d, J=8.4 Hz, 2H), 2.19-2.11 (m, 2H), 2.07-1.97 (m, 2H

### General Procedure for Preparation of Intermediate 6:

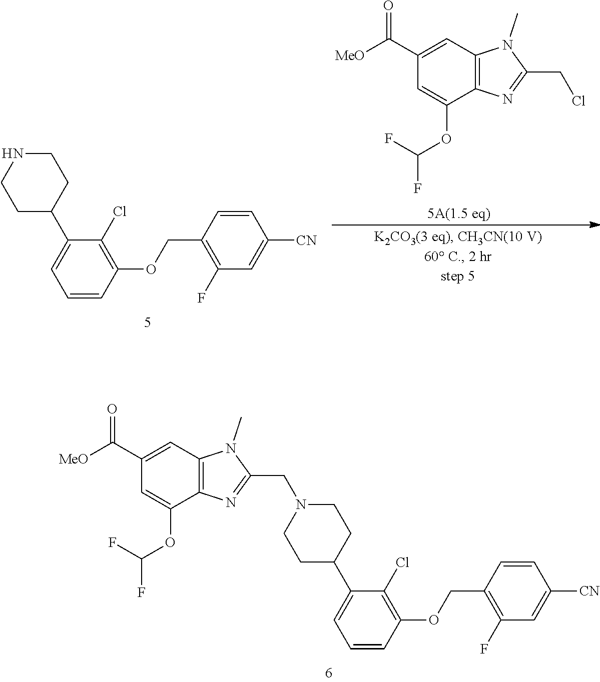 [SMILES: N#Cc1ccc(COc2cccc(C3CCNCC3)c2Cl)c(F)c1 ; COC(=O)c1cc(OC(F)F)c2nc(CCl)n(C)c2c1 ; COC(=O)c1cc(OC(F)F)c2nc(CN3CCC(c4cccc(OCc5ccc(C#N)cc5F)c4Cl)CC3)n(C)c2c1]

To a solution of 5 (120 mg, 261.53 μmol, 1 eq, TFA) in CH₃CN (1.2 mL) was added K₂CO₃ (108.44 mg, 784.60 μmol, 3 eq) and 5A (79.68 mg, 261.53 μmol, 1 eq). The mixture was stirred at 60° C. for 2 hr. LCMS showed 5 was consumed completely and one main peak with desired mass was detected. The reaction mixture was diluted with H₂O 5 mL and extracted with EtOAc 20 mL (10 mL*2). The combined organic layers were washed with brine 10 mL, dried over Na₂SO₄, filtered, and concentrated under reduced pressure to give a residue. The residue was purified by column chromatography (SiO₂, PE/EtOAc=1/0 to 1/1). 6 (150 mg, 230.01 μmol, 87.95% yield) was obtained as a white solid.

LCMS: RT=1.582 min, MS cal.: 612.2/613.2, [M+H]⁺=613.0/615.0

¹HNMR (400 MHz, CHCl3-d) δ=7.99 (s, 1H), 7.85 (br t, J=7.6 Hz, 1H), 7.72 (s, 1H), 7.54 (br d, J=8.0 Hz, 1H), 7.40 (br d, J=9.6 Hz, 1H), 7.32 (t, J=74.4 Hz 1H), 7.24-7.18 (m, 1H), 6.97 (br d, J=7.6 Hz, 1H), 6.86 (br d, J=8.0 Hz, 1H), 5.24 (s, 2H), 4.13 (q, J=7.2 Hz, 1H), 4.04-3.90 (m, 8H), 3.20-3.10 (m, 1H), 3.08-2.98 (m, 1H), 2.41 (br dd, J=2.8, 11.6 Hz, 1H), 2.05 (s, 1H), 1.90 (br d, J=11.2 Hz, 2H), 1.60 (br s, 5H)

### General Procedure for Preparation of Compound 179 (7):

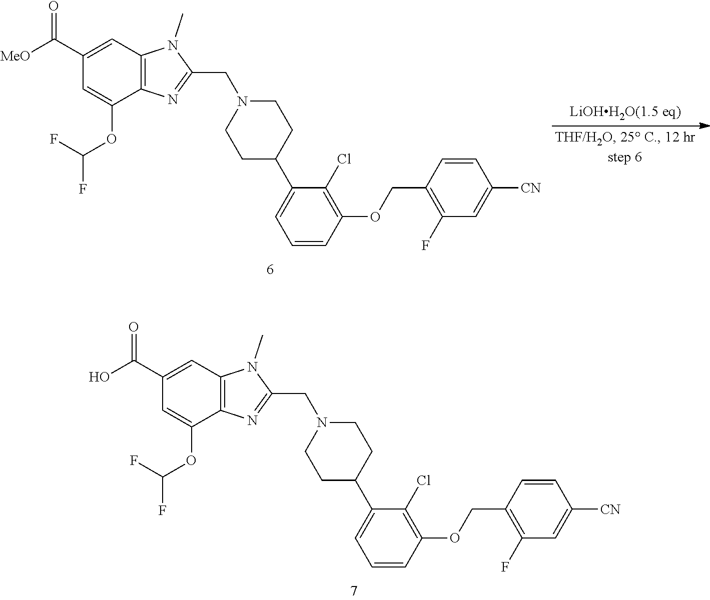 [SMILES: COC(=O)c1cc(OC(F)F)c2nc(CN3CCC(c4cccc(OCc5ccc(C#N)cc5F)c4Cl)CC3)n(C)c2c1 ; Cn1c(CN2CCC(c3cccc(OCc4ccc(C#N)cc4F)c3Cl)CC2)nc2c(OC(F)F)cc(C(=O)O)cc21]

To a solution of 6 (70 mg, 114.19 μmol, 1 eq) in THF (0.7 mL) and H₂O (0.3 mL) was added

LiOH·H2O (7.19 mg, 171.28 μmol, 1.5 eq). The mixture was stirred at 25° C. for 12 hr. LCMS showed 8% of 6 remained. Several new peaks were shown on LCMS and 84% of desired product was detected. The reaction mixture was adjusted to pH=7 with 0.5M citric acid. The residue was purified by prep-HPLC (neutral condition: column: Waters Xbridge Prep OBD C18 150*40 mm*10 um; mobile phase: [H₂O (10 mM NH₄HCO₃)-ACN]; gradient: 25%-55% B over 8.0 min). 2-((4-(2-Chloro-3-((4-cyano-2-fluorobenzyl)oxy)phenyl)piperidin-1-yl)methyl)-4-(difluoromethoxy)-1-methyl-1H-benzo[d]imidazole-6-carboxylic acid (28.88 mg, 47.25 μmol, 41.38% yield) was obtained as a white solid.

LCMS: RT=2.675 min, MS cal.: 598.2/599.2, [M+H]⁺=599.2/600.2

HPLC: RT=10.993 min

¹H NMR (400 MHz, MeOH-d4) δ=8.10 (s, 1H), 7.87-7.81 (m, 1H), 7.68 (s, 1H), 7.65-7.57 (m, 2H), 7.23 (t, J=74 Hz, 1H), 7.26-7.22 (t, J=8.4 Hz, 1H), 7.01 (dd, J=2.0, 8.0 Hz, 2H), 5.28 (s, 2H), 4.02 (s, 3H), 3.95 (s, 2H), 3.18-3.11 (m, 1H), 3.08 (br d, J=11.6 Hz, 2H), 2.44-2.35 (m, 2H), 1.89-1.72 (m, 4H)

### Example A179—Preparation of Compound 181. 2-((4-(5-((4-Cyano-2-fluorobenzyl)oxy)-2-fluorophenyl)piperidin-1-yl)methyl)-4-(difluoromethoxy)-1-methyl-1H-benzo[d]imidazole-6-carboxylic acid (Compound 181) (7)

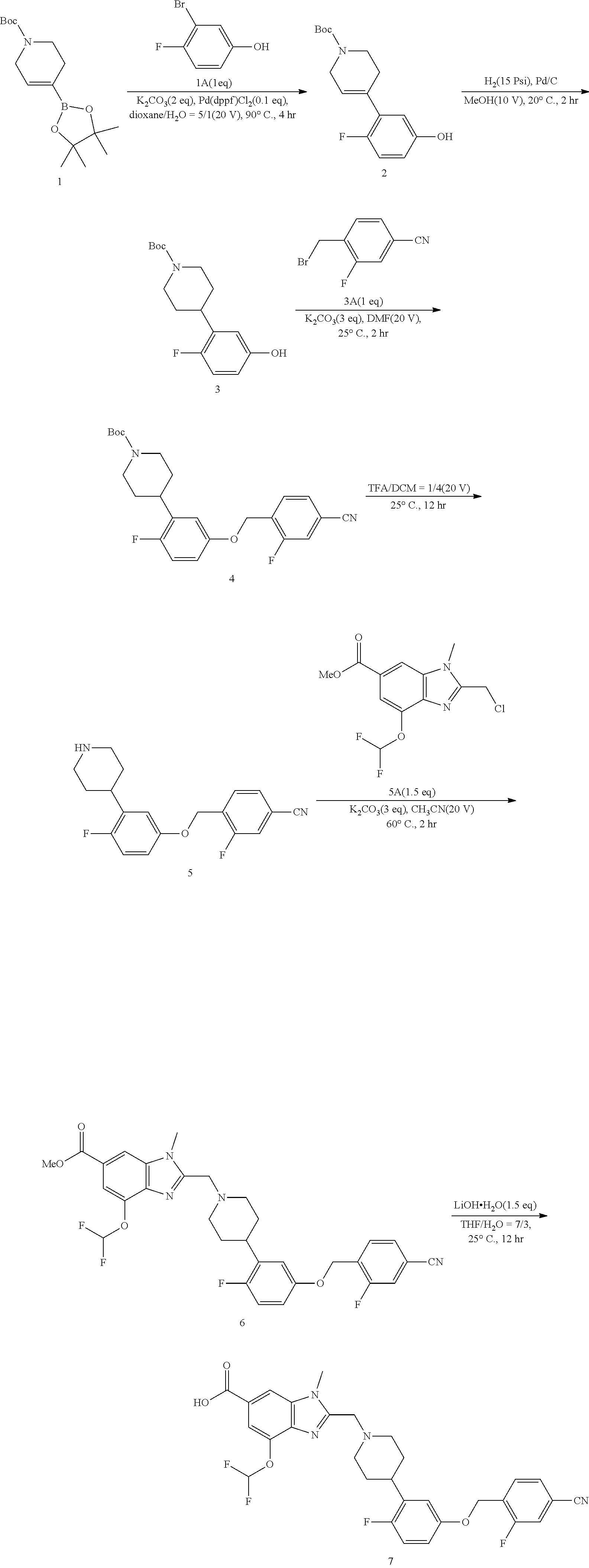

### General Procedure for Preparation of Intermediate 2:

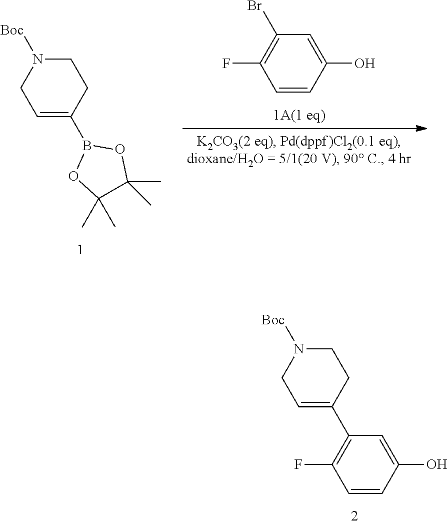 [SMILES: CC(C)(C)OC(=O)N1CC=C(B2OC(C)(C)C(C)(C)O2)CC1 ; Oc1ccc(F)c(Br)c1 ; CC(C)(C)OC(=O)N1CC=C(c2cc(O)ccc2F)CC1]

A mixture of 1 (1 g, 3.23 mmol, 1 eq), 1A (617.70 mg, 3.23 mmol, 1 eq), K₂CO₃ (893.93 mg, 6.47 mmol, 2 eq), Pd(dppf)Cl₂ (236.64 mg, 323.41 μmol, 0.1 eq) in dioxane (10 mL) and H₂O (2 mL) was degassed and purged with N₂ for 3 times, and then the mixture was stirred at 90° C. for 4 hr under N₂ atmosphere. TLC (PE:EtOAc=3:1, R_{f}=0.5) indicated 1 was consumed completely and one new spot formed. The reaction was clean according to TLC. The suspension was filtered through a pad of Celite, and filter cake was washed with EtOAc (10 mL*3). The combined filtrates were diluted with H₂O 20 mL and partitioned between EtOAc and H₂O. The aqueous phase was extracted with EtOAc (15 mL*2). The combined organic layers were washed with brine 15 mL, dried over Na₂SO₄, filtered, and concentrated under reduced pressure to give a residue. The residue was purified by column chromatography (SiO₂, Petroleum ether/Ethyl acetate=10/1 to 5/1). 2 (750 mg, 2.56 mmol, 79.06% yield) was obtained as a white solid.

LCMS: RT=1.237 min, MS cal.: 293.1, [M−55]⁺=238.0

¹H NMR (400 MHz, CDCl₃) δ=6.90 (dd, J=8.8, 10.4 Hz, 1H), 6.76-6.67 (m, 2H), 5.94 (br s, 2H), 4.07 (br d, J=2.4 Hz, 2H), 3.63 (br t, J=5.6 Hz, 2H), 2.54-2.46 (m, 2H), 1.53-1.48 (m, 9H)

### General Procedure for Preparation of Intermediate 3:

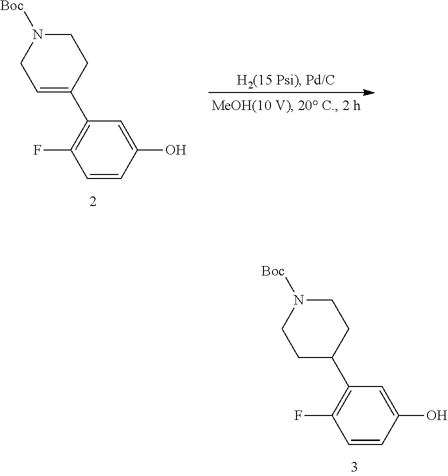 [SMILES: CC(C)(C)OC(=O)N1CC=C(c2cc(O)ccc2F)CC1 ; CC(C)(C)OC(=O)N1CCC(c2cc(O)ccc2F)CC1]

MeOH (5 mL) was charged to the 100 mL necked round bottom flask, then Pd/C (50 mg) was added at 25° C. within 1 min under N₂ atmosphere. At 25° C. inner temperature, 2 (500 mg, 1.70 mmol, 1 eq) was added to the reaction mixture at 25° C. within 1 min and stirred at 25° C. for 2 hr under H₂ atmosphere. LC-MS showed 2 was consumed completely and one main peak with desired m/z was detected. The mixture was filtered by diatomite. The filter cake was washed by MeOH (30 mL), the combined organic phase was filtered and concentrated in vacuum. 3 (500 mg, 1.51 mmol, 88.69% yield, 89.3% purity) was obtained as a white solid.

LCMS: RT=1.254 min, MS cal.: 295.0, [M+H]⁺=294.0

1H NMR (400 MHz, CHCl3-d) δ=7.62 (t, J=7.6 Hz, 1H), 7.52 (t, J=7.6 Hz, 1H), 7.43 (dd, J=1.2, 8.0 Hz, 1H), 7.36 (dd, J=1.6, 9.2 Hz, 1H), 6.82 (d, J=7.6 Hz, 1H), 6.64 (d, J=8.0 Hz, 1H), 5.49 (s, 2H), 3.32 (br s, 1H), 2.72 (br t, J=8.8 Hz, 1H), 2.05-1.92 (m, 2H), 1.84-1.68 (m, 6H)

### General Procedure for Preparation of Intermediate 4:

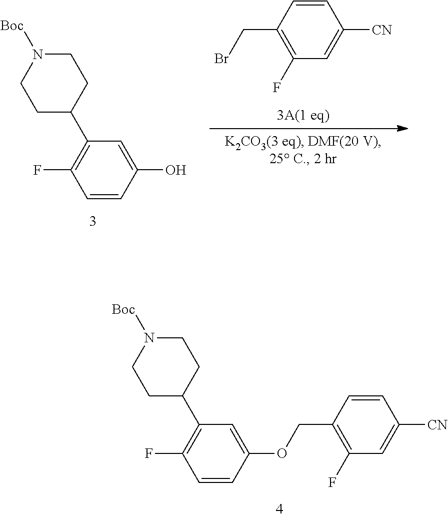 [SMILES: CC(C)(C)OC(=O)N1CCC(c2cc(O)ccc2F)CC1 ; N#Cc1ccc(CBr)c(F)c1 ; CC(C)(C)OC(=O)N1CCC(c2cc(OCc3ccc(C#N)cc3F)ccc2F)CC1]

To a solution of 3 (200 mg, 677.17 μmol, 1 eq) in DMF (2 mL) was added K₂CO₃ (280.76 mg, 2.03 mmol, 3 eq) and 3A (144.94 mg, 677.17 μmol, 1 eq). The mixture was stirred at 25° C. for 2 hr. LC-MS (product RT=1.585 min) showed 3A was consumed completely and desired mass was detected. The reaction mixture was diluted with H₂O 20 mL and extracted with EtOAc 60 mL (20 mL*3). The combined organic layers were washed with brine 20 mL, dried over Na₂SO₄, filtered, and concentrated under reduced pressure to give a residue. The residue was purified by column chromatography (SiO₂, Petroleum ether/Ethyl acetate=20/1 to 3/1). 4 (250 mg, 583.47 μmol, 86.16% yield) was obtained as a colorless oil.

LCMS: RT=1.584 min MS cal: 428.2 [M−55]⁺=373.0

¹H NMR (400 MHz, CDCl₃) δ=7.69 (t, J=7.6 Hz, 1H), 7.51 (d, J=8.0 Hz, 1H), 7.41 (d, J=9.6 Hz, 1H), 6.97 (t, J=9.2 Hz, 1H), 6.82 (dd, J=2.8, 5.6 Hz, 1H), 6.75 (td, J=3.6, 8.4 Hz, 1H), 5.13 (s, 2H), 4.26 (br d, J=13.2 Hz, 2H), 2.99 (tt, J=3.2, 12.0 Hz, 1H), 2.83 (br t, J=12.4 Hz, 2H), 1.81 (br d, J=12.8 Hz, 2H), 1.70-1.53 (m, 4H), 1.49 (s, 9H)

### General Procedure for Preparation of Intermediate 5:

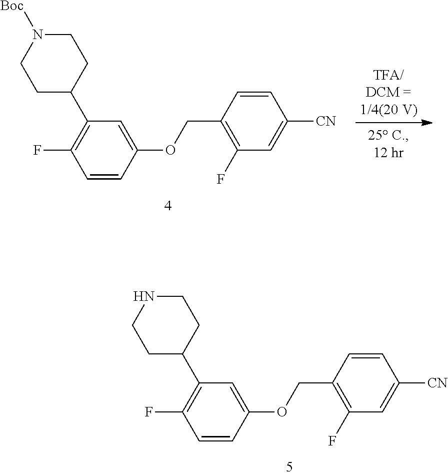 [SMILES: CC(C)(C)OC(=O)N1CCC(c2cc(OCc3ccc(C#N)cc3F)ccc2F)CC1 ; N#Cc1ccc(COc2ccc(F)c(C3CCNCC3)c2)c(F)c1]

A mixture of 4 (220 mg, 513.45 μmol, 1 eq), TFA (767.50 mg, 6.73 mmol, 0.5 mL, 13.11 eq) in DCM (2 mL) was degassed and purged with N₂ for 3 times, and then the mixture was stirred at 25° C. for 12 hr under N₂ atmosphere. LC-MS (product RT=1.026 min) showed 4 was consumed completely and desired mass was detected. The reaction mixture was concentrated under reduced pressure to give a residue. 5 (220 mg, 497.31 μmol, 96.86% yield, TFA) was obtained as a colorless oil.

LCMS: RT=1.026 min, MS cal.: 328.1, [M+H]⁺=329.0

¹H NMR (400 MHz, CDCl₃) δ=7.73-7.66 (m, 1H), 7.54-7.49 (m, 1H), 7.41 (dd, J=1.2, 9.2 Hz, 1H), 7.01 (t, J=9.2 Hz, 1H), 6.87-6.83 (m, 2H), 5.14 (s, 2H), 3.65 (br d, J=11.2 Hz, 2H), 3.14 (br d, J=7.6 Hz, 3H), 2.19-1.97 (m, 4H)

### General Procedure for Preparation of Intermediate 6:

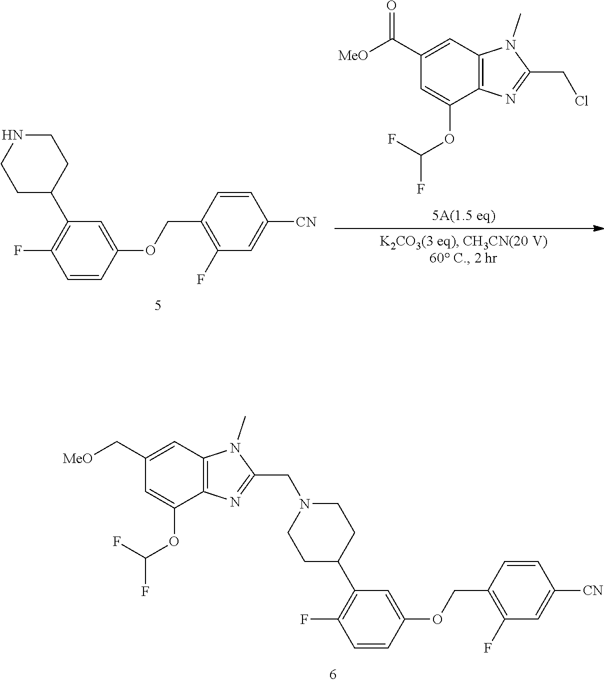 [SMILES: N#Cc1ccc(COc2ccc(F)c(C3CCNCC3)c2)c(F)c1 ; COC(=O)c1cc(OC(F)F)c2nc(CCl)n(C)c2c1 ; COCc1cc(OC(F)F)c2nc(CN3CCC(c4cc(OCc5ccc(C#N)cc5F)ccc4F)CC3)n(C)c2c1]

A mixture of 5 (220 mg, 497.31 μmol, 1 eq, TFA), 5A (151.52 mg, 497.31 μmol, 1 eq), K₂CO₃ (206.19 mg, 1.49 mmol, 3 eq) in CH₃CN (2 mL) was degassed and purged with N₂ for 3 times, and then the mixture was stirred at 60° C. for 2 hr under N₂ atmosphere. LC-MS showed 5 was consumed completely and desired mass was detected. The reaction mixture was with H₂O 20 mL and extracted with EtOAc 45 mL (15 mL*3). The combined organic layers were washed with brine 15 mL, dried over Na₂SO₄, filtered, and concentrated under reduced pressure to give a residue. The residue was purified by column chromatography (SiO₂, Petroleum ether/Ethyl acetate=10/1 to 1/1). 6 (240 mg, 402.30 μmol, 80.89% yield) was obtained as a yellow oil.

LCMS: RT=1.570 min, MS cal.: 596.2, [M+H]⁺=597.1

1H NMR (400 MHz, CDCl₃) δ=7.99 (s, 1H), 7.74-7.65 (m, 2H), 7.50 (d, J=8.4 Hz, 1H), 7.40 (d, J=9.2 Hz, 1H), 7.31 (t, J=74.4 Hz, 1H), 6.96 (t, J=9.2 Hz, 1H), 6.84 (dd, J=2.8, 5.6 Hz, 1H), 6.74 (td, J=3.6, 8.4 Hz, 1H), 5.13 (s, 2H), 4.04-3.95 (m, 6H), 3.91 (br s, 2H), 3.10-2.82 (m, 3H), 2.36 (br s, 2H), 1.90-1.83 (m, 2H), 1.83-1.69 (m, 2H)

### General Procedure for Preparation of Compound 181 (7):

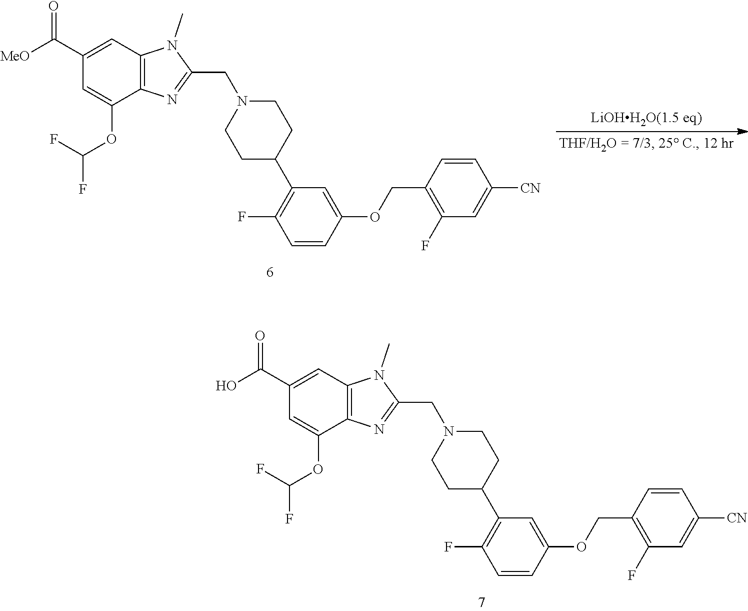 [SMILES: COC(=O)c1cc(OC(F)F)c2nc(CN3CCC(c4cc(OCc5ccc(C#N)cc5F)ccc4F)CC3)n(C)c2c1 ; Cn1c(CN2CCC(c3cc(OCc4ccc(C#N)cc4F)ccc3F)CC2)nc2c(OC(F)F)cc(C(=O)O)cc21]

A mixture of 6 (150 mg, 251.44 μmol, 1 eq) LiOH·H₂O (15.83 mg, 377.16 μmol, 1.5 eq) in H₂O (0.6 mL) and THF (1.4 mL) was degassed and purged with N₂ for 3 times, and then the mixture was stirred at 25° C. for 12 hr under N₂ atmosphere. LC-MS (RT=1.612 min) showed 6 was consumed completely and desired mass was detected. The reaction was purified directly. 2-((4-(5-((4-Cyano-2-fluorobenzyl)oxy)-2-fluorophenyl)piperidin-1-yl)methyl)-4-(difluoromethoxy)-1-methyl-1H-benzo[d]imidazole-6-carboxylic acid (28.13 mg, 47.69 μmol, 18.97% yield, 98.77% purity) was obtained as a white solid.

LCMS: RT=2.383 min, MS cal.: 582.2, [M+H]⁺=583.2

HPLC: RT=11.01 min, purity: 98.77%

¹HdMR (400 MHz, MEOH-d4) δ=8.10 (d, J=1.2 Hz, 1H), 7.78-7.70 (m, 1H), 7.68 (s, 1H), 7.63-7.56 (m, 2H), 7.24 (t, J=74.4 Hz, 1H), 7.00-6.94 (m, 1H), 6.93-6.89 (m, 1H), 6.86-6.78 (m, 1H), 5.18 (s, 2H), 4.02 (s, 3H), 3.94 (s, 2H), 3.06 (br d, J=11.6 Hz, 2H), 2.88 (br t, J=7.2 Hz, 1H), 2.44-2.32 (m, 2H), 1.85-1.76 (m, 4H)

### Example A180—Preparation of Compound 182. 2-((4-(2-((4-Cyano-2-fluorobenzyl)oxy)-3-fluorophenyl)piperidin-1-yl)methyl)-4-(difluoromethoxy)-1-methyl-1H-benzo[d]imidazole-6-carboxylic acid (Compound 182) (7)

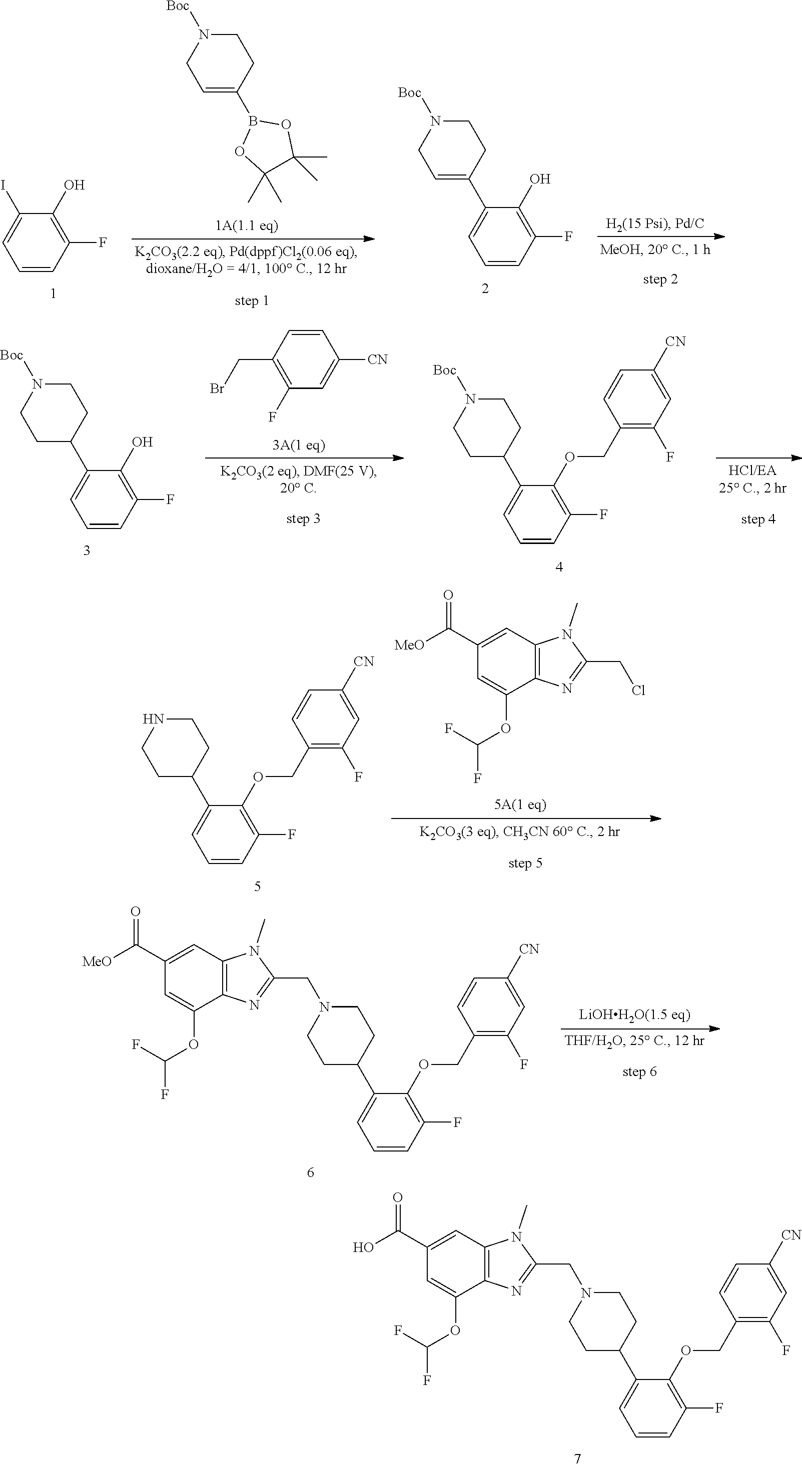

### General Procedure for Preparation of Intermediate 2:

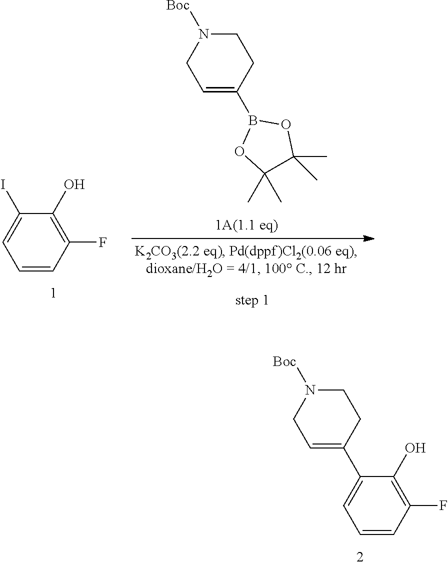 [SMILES: Oc1c(F)cccc1I ; CC(C)(C)OC(=O)N1CC=C(B2OC(C)(C)C(C)(C)O2)CC1 ; CC(C)(C)OC(=O)N1CC=C(c2cccc(F)c2O)CC1]

To a solution of 1 (3 g, 12.61 mmol, 1 eq) in dioxane (48 mL) and H₂O (12 mL) was added 1A (4.29 g, 13.87 mmol, 1.1 eq), K₂CO₃ (3.83 g, 27.73 mmol, 2.2 eq) and Pd(dppf)Cl₂ (553.40 mg, 756.31 μmol, 0.06 eq). The mixture was degassed and purged with N₂ for 3 times, and then the mixture was stirred at 100° C. for 16 hr under N₂ atmosphere. TLC (PE/EA=5/1, Rf=0.35) indicated 1 was consumed completely and one new spot was formed. The reaction was clean according to TLC. The mixture was filtered by celite pad, and the filtrate was added water (100 mL), extracted with EA (100 mL*3), the organic phase was washed with brine (100 mL), filtered, and concentrated in vacuum to get a residue. The residue was purified by column chromatography (SiO₂, Petroleum ether/Ethyl acetate=1/0 to 20/1). 2 (3.2 g, 10.91 mmol, 86.54% yield) was obtained as a yellow solid.

LCMS: RT=0.513 min, MS cal.: 293.1, [M−55]=238.0

¹H NMR (400 MHz, DMSO-d₆) δ=9.48 (s, 1H), 7.06 (br t, J=8.0 Hz, 1H), 6.90 (br d, J=8.0 Hz, 1H), 6.82-6.71 (m, 1H), 5.85 (br s, 1H), 3.96 (br s, 2H), 3.49 (br t, J=4.0 Hz, 2H), 2.42 (br s, 2H), 1.43 (s, 9H)

### General Procedure for Preparation of Intermediate 3:

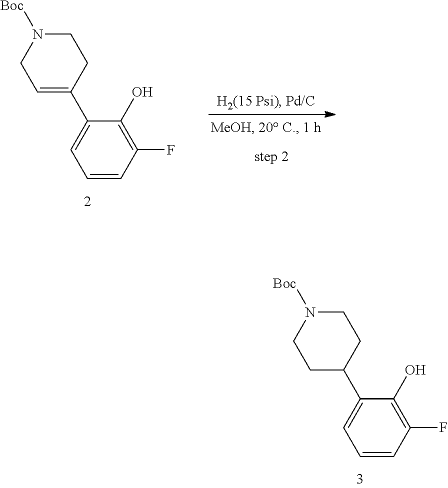 [SMILES: CC(C)(C)OC(=O)N1CC=C(c2cccc(F)c2O)CC1 ; CC(C)(C)OC(=O)N1CCC(c2cccc(F)c2O)CC1]

To a solution of 2 (3.2 g, 10.91 mmol, 1 eq) in THF (65 mL) was added Pd/C (1.6 g, 1.50 mmol, 10% purity, 1.38e-1 eq) under N₂ atmosphere. The suspension was degassed and purged with H₂ for 3 times. The mixture was stirred under H₂ (15 Psi) at 25° C. for 1 hr. LCMS showed 2 was consumed completely and one main peak with desired mass was detected. The mixture was filtered by celite pad, and the filtrate was concentrated in vacuum to get a residue. The crude product was triturated with MTBE and filtered, filter-cake was collected and concentrated in vacuum to get the product. 3 (2.05 g, 0.94 mmol, 63.63% yield) was obtained as a light yellow solid. LCMS: RT=0.548 min, MS cal.: 295.1, [M−55]=240.2

¹H NMR (400 MHz, DMSO-d₆) δ=9.42 (s, 1H), 7.03-6.95 (m, 1H), 6.92 (br d, J=8.0 Hz, 1H), 6.79-6.71 (m, 1H), 4.06 (br d, J=12.0 Hz, 2H), 3.04 (br t, J=12.0 Hz, 1H), 2.80 (br s, 2H), 1.69 (br d, J=16.0 Hz, 2H), 1.53-1.43 (m, 2H), 1.41 (s, 9H)

### General Procedure for Preparation of Intermediate 4:

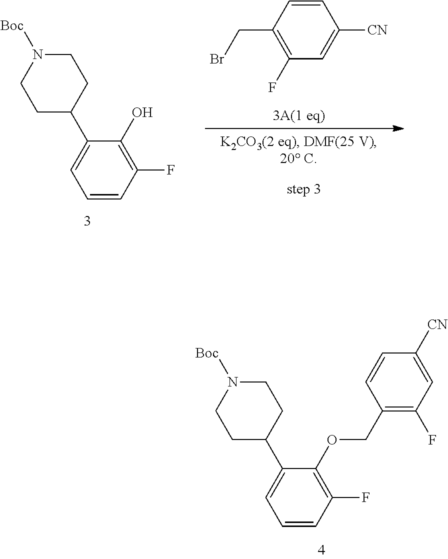 [SMILES: CC(C)(C)OC(=O)N1CCC(c2cccc(F)c2O)CC1 ; N#Cc1ccc(CBr)c(F)c1 ; CC(C)(C)OC(=O)N1CCC(c2cccc(F)c2OCc2ccc(C#N)cc2F)CC1]

To a solution of 3 (200 mg, 677.17 μmol, 1 eq) and 3A (144.94 mg, 677.17 μmol, 1 eq) in DMF (5 mL) was added K₂CO₃ (187.18 mg, 1.35 mmol, 2 eq). The mixture was stirred at 20° C. for 12 hr under N₂ atmosphere. LC-MS showed 3 was consumed completely and one main peak with desired mass was detected. The reaction mixture was quenched by addition water (10 mL) and extracted with EA (10 mL*3). The combined organic layers were washed with brine (30 mL), dried over Na₂SO₄, filtered, and concentrated under reduced pressure to give a residue. The residue was purified by column chromatography (SiO₂, Petroleum ether/Ethyl acetate=I/O to 20/1) to get 4 (200 mg, 466.78 μmol, yield 68.93%) as a yellow oil.

LCMS: RT=0.644 min, MS cal.: 428.1, [M+Na]=451.2

¹H NMR (400 MHz, DMSO-d₆) δ=7.94 (d, J=8.0 Hz, 1H), 7.79-7.74 (m, 2H), 7.18-7.02 (m, 3H), 5.18 (s, 2H), 4.04-3.97 (m, 2H), 2.98-2.85 (m, 1H), 2.67 (br d, J=4.0 Hz, 2H), 1.48-1.41 (m, 4H), 1.40 (s, 9H)

### General Procedure for Preparation of Intermediate 5:

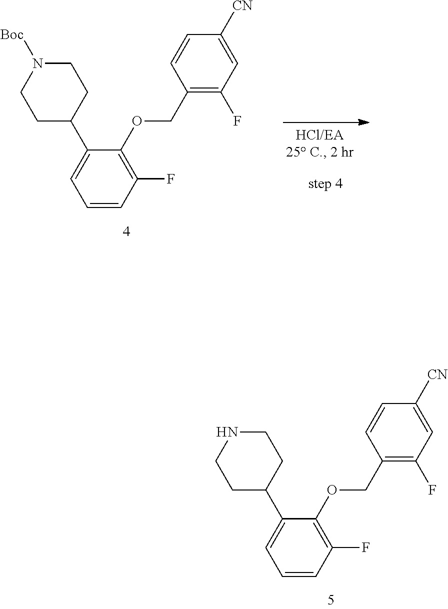 [SMILES: CC(C)(C)OC(=O)N1CCC(c2cccc(F)c2OCc2ccc(C#N)cc2F)CC1 ; N#Cc1ccc(COc2c(F)cccc2C2CCNCC2)c(F)c1]

To a solution of 4 (200 mg, 466.78 μmol, 1 eq) in HCl/EtOAc (5 mL). The mixture was stirred at 20° C. for 1 hr. LC-MS showed 4 was consumed completely and one main peak with desired mass was detected. The reaction mixture was concentrated under reduced pressure to get a residue. Then adjusted pH=8 bp Na₂CO₃, extracted with EA (5 mL), dried over Na₂SO₄, filtered, and concentrated in vacuum to get the crude product. The crude product was not purified and used to next step directly. 5 (160 mg, crude) was obtained as a yellow oil.

LCMS: ET78548-35-P1B1, RT=0.387 min, MS cal.: 328.1, [M+H]⁺=329.2

### General Procedure for Preparation of Intermediate 6:

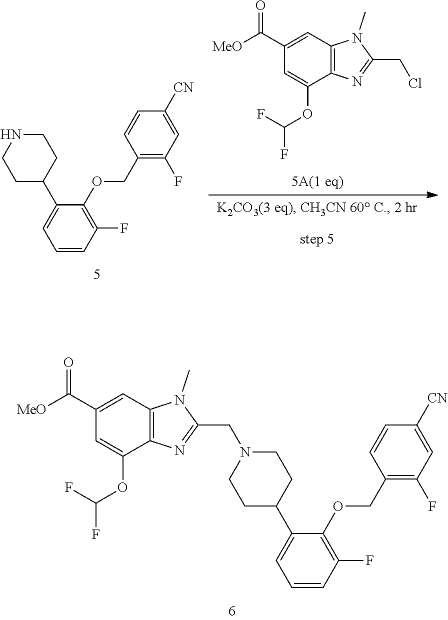 [SMILES: N#Cc1ccc(COc2c(F)cccc2C2CCNCC2)c(F)c1 ; COC(=O)c1cc(OC(F)F)c2nc(CCl)n(C)c2c1 ; COC(=O)c1cc(OC(F)F)c2nc(CN3CCC(c4cccc(F)c4OCc4ccc(C#N)cc4F)CC3)n(C)c2c1]

To a solution of 5 (100 mg, 304.55 μmol, 1 eq) and Compound 5A (92.79 mg, 304.55 μmol, 1 eq.) in CH₃CN (5 mL) was added K₂CO₃ (126.27 mg, 913.65 μmol, 3 eq). The mixture was stirred at 60° C. for 2 hr. LC-MS showed 5 was consumed completely and desired mass was detected. The reaction mixture was quenched by addition water (10 Ml) and extracted with EA (10 mL*3). The combined organic layers were washed with brine (10 mL), dried over Na₂SO₄, filtered, and concentrated under reduced pressure to give a residue. The residue was purified by Prep-TLC (SiO₂, PE:EA=1:1). 6 (130 mg, 217.91 μmol) was obtained as a yellow solid.

LCMS: Rt=1.843 min, MS cal.: 596.2, [M+Na]=619.3

¹H NMR (400 MHz, DMSO-d₆) δ=8.11 (d, J=1.2 Hz, 1H), 7.98-7.92 (m, 1H), 7.80-7.71 (m, 2H), 7.64 (t, J=76.4 1H), 7.53 (s, 1H), 7.16-7.03 (m, 3H), 5.16 (s, 2H), 3.95 (s, 3H), 3.90 (s, 3H), 3.84 (s, 2H), 2.90 (br d, J=12.0 Hz, 2H), 2.82-2.72 (m, 1H), 2.14-2.06 (m, 2H), 1.62-1.51 (m, 2H), 1.49-1.41 (m, 2H)

### General Procedure for Preparation of Compound 182 (7):

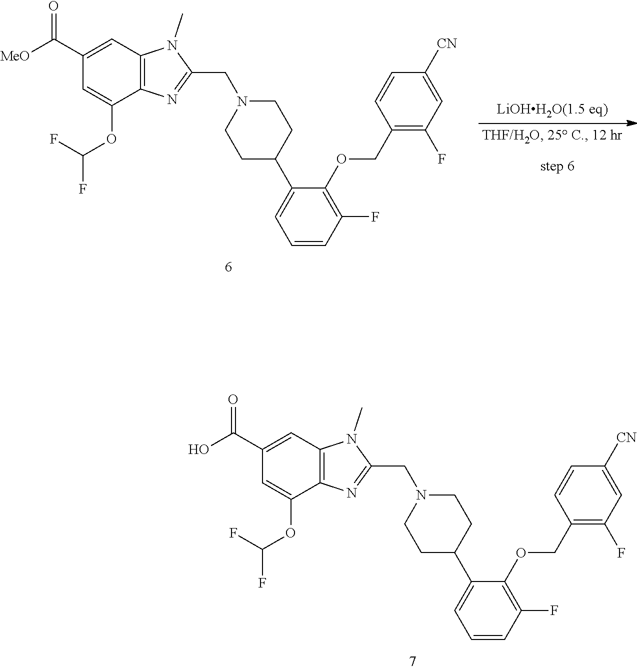 [SMILES: COC(=O)c1cc(OC(F)F)c2nc(CN3CCC(c4cccc(F)c4OCc4ccc(C#N)cc4F)CC3)n(C)c2c1 ; Cn1c(CN2CCC(c3cccc(F)c3OCc3ccc(C#N)cc3F)CC2)nc2c(OC(F)F)cc(C(=O)O)cc21]

To a solution of 6 (100 mg, 167.62 μmol, 1 eq) in THF (1 mL) and H₂O (1 mL) was added LiOH·H₂O (10.55 mg, 251.44 μmol, 1.5 eq). The mixture was stirred at 25° C. for 12 hr. LC-MS showed 6 was consumed completely and one main peak with desired mass was detected. The mixture was adjusted pH=7 by citric acid. The residue was purified by prep-HPLC (column: Waters Xbridge Prep OBD C18 150*40 mm*10 um; mobile phase: [H₂O (10 mM NH₄HCO₃)-ACN]; gradient: 30%-60% B over 8.0 min). 2-((4-(2-((4-Cyano-2-fluorobenzyl)oxy)-3-fluorophenyl)piperidin-1-yl)methyl)-4-(difluoromethoxy)-1-methyl-1H-benzo[d]imidazole-6-carboxylic acid (46.45 mg, 77.50 μmol, 46.24% yield, 97.2% purity) was obtained as a white solid.

LCMS: Rt=2.401 min, MS cal.: 582.1, [M+1]⁺=583.2

HPLC: Rt=2.652 min

¹H NMR (400 MHz, DMSO-d₆) δ=8.04 (s, 1H), 7.95 (d, J=8.0, 1H), 7.82-7.71 (m, 2H), 7.74 (t, J=74.4 1H), 7.61 (s, 1H), 7.17-7.02 (m, 3H), 5.16 (s, 2H), 3.93 (s, 3H), 3.82 (s, 2H), 2.90 (br d, J=8.0, 2H), 2.82-2.71 (m, 1H), 2.09 (br t, J=12.0 2H), 1.64-1.51 (m, 2H), 1.50-1.40 (m, 2H)

### Example A181—Preparation of Compound 183. 2-((4-(6-((4-Cyano-2-fluorobenzyl)oxy)pyridin-2-yl)piperidin-1-yl)methyl)-4-(2-fluoroethoxy)-1-methyl-1H-benzo[d]imidazole-6-carboxylic acid (Compound 183) (8)

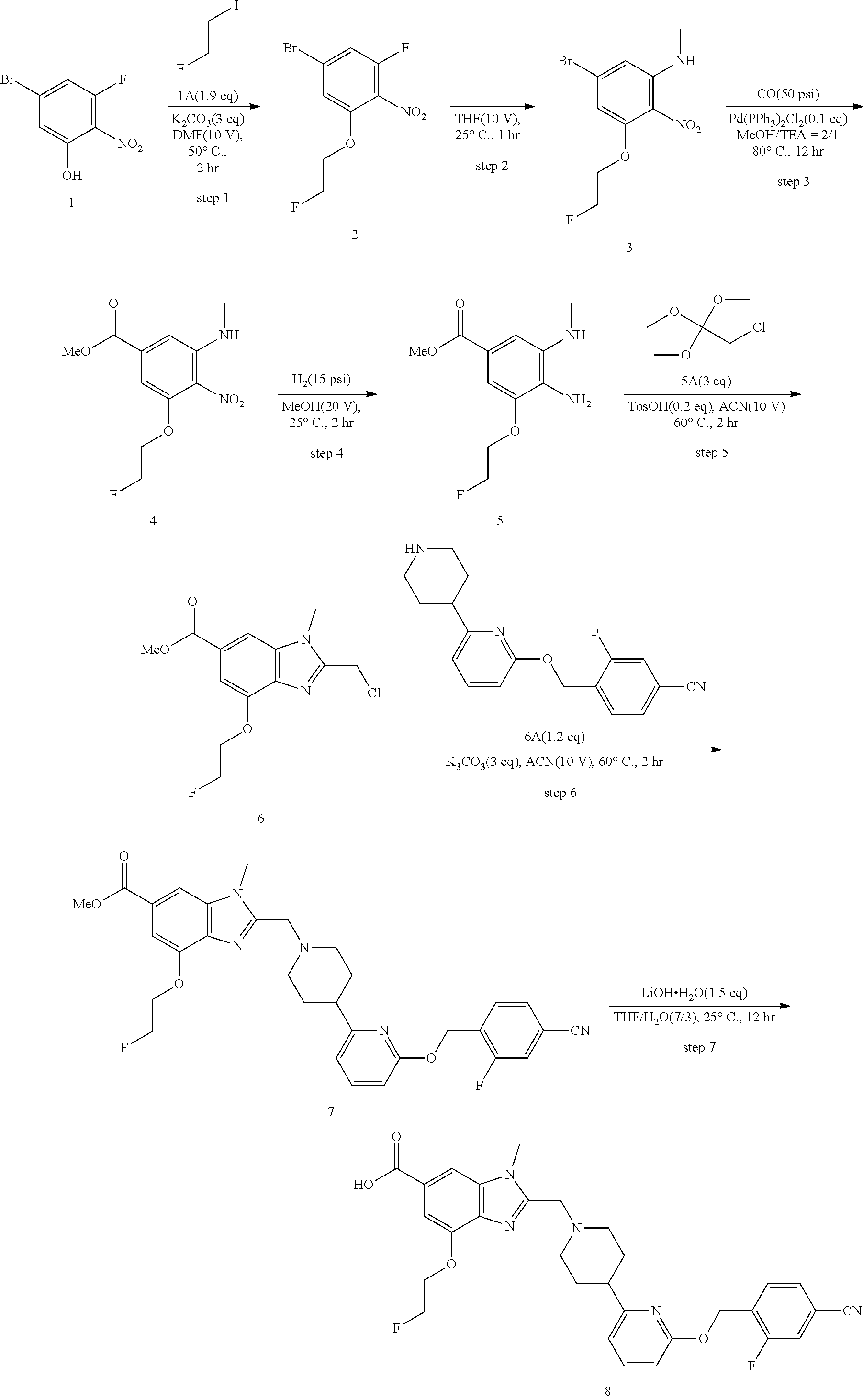

### General Procedure for Preparation of Intermediate 2:

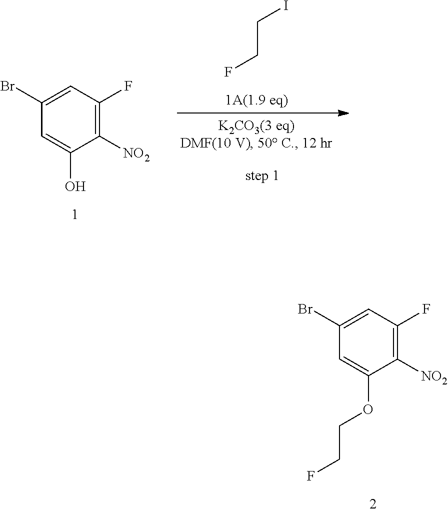

To a solution of 1 (2 g, 8.47 mmol, 1 eq) in DMF (20 mL) was added K₂CO₃ (3.51 g, 25.42 mmol, 3 eq) and 1A (2.80 g, 16.10 mmol, 1.9 eq) at 25° C. The mixture was stirred at 50° C. for 12 hr. TLC showed 1 was consumed completely. The reaction mixture was diluted with H₂O (40) mL and extracted with EtOAc (30 mL). The combined organic layers were washed with brine 40 mL, dried over Na₂SO₄, filtered and concentrated under reduced pressure to give a residue. The residue was purified by column chromatography (SiO₂, Petroleum ether/Ethyl acetate=1/0 to 0/1). 2 (2.2 g, 7.80 mmol, 92.04% yield) was obtained as a yellow solid.

¹H NMR (400 MHz, CHCl3-d) δ=7.09 (d, J=8.8 Hz, 1H), 7.05 (s, 1H), 4.80 (dt, J=47.2, 4.0 Hz, 2H), 4.38 (dt, J=22.8, 4.0 Hz, 2H)

### General Procedure for Preparation of Intermediate 3:

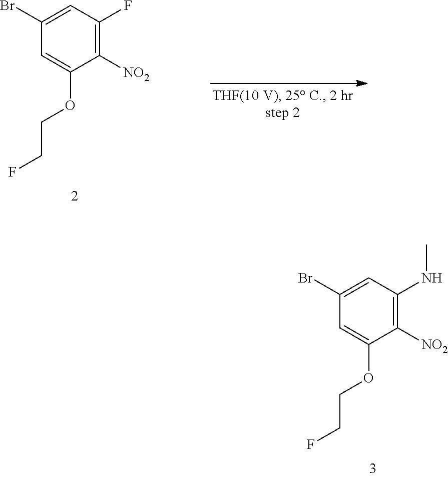 [SMILES: O=[N+]([O-])c1c(F)cc(Br)cc1OCCF ; CNc1cc(Br)cc(OCCF)c1[N+](=O)[O-]]

To a solution of 2 (1.6 g, 5.67 mmol, 1 eq) in THF (16 mL) was added methylamine (2.06 g, 19.86 mmol, 30% purity, 3.5 eq) at 25° C. Then the mixture was stirred at 25° C. for 2 hr. LC-MS showed 2 was consumed completely and desired mass was detected. The reaction mixture was added FA (1 M) to pH=5 at 25° C., then the mixture was diluted with H₂O (30 mL), and then extracted with EtOAc (20 mL×2). The combined organic layers were washed with brine (20 mL×2), dried over Na₂SO₄, filtered and concentrated under reduced pressure to give 3 (2 g, crude) as a red solid.

LCMS: RT=0.785 min, MS cal.: 292.0, [M−H]⁻=291.1

¹H NMR (400 MHz, CHCl3-d) δ=6.58 (d, J=1.6 Hz, 1H), 6.43 (d, J=1.6 Hz, 1H), 4.81 (dt, J=47.2, 4.0 Hz, 2H), 4.31 (dt, J=26.4, 4.0 Hz, 2H), 2.92 (s, 3H)

### General Procedure for Preparation of Intermediate 4:

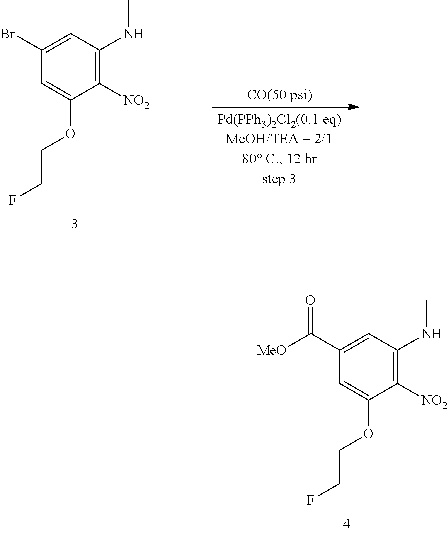 [SMILES: CNc1cc(Br)cc(OCCF)c1[N+](=O)[O-] ; CNc1cc(C(=O)OC)cc(OCCF)c1[N+](=O)[O-]]

1 (1 g, 3.41 mmol, 1 eq) was charged to the round bottom flask, then starting TEA (5 mL) and MeOH (10 mL), Pd(PPh₃)₂Cl₂ (239.48 mg, 341.19 μmol, 0.1 eq) was added to the mixture at 25° C. After the addition, the mixture was stirred at 80° C. for 12 h under CO (50 psi). LC-MS (product Rt=1.115) showed 1 was consumed completely and desired mass was detected. The reaction mixture was filtered. The reaction mixture was adjusted to pH 5 with 1M HCl. The aqueous layer was extracted with Ethyl acetate (10 mL*3). The combined organic phase was washed with brine (10 mL*2), dried with anhydrous Na₂SO₄, filtered, and concentrated in vacuum. The residue was purified by column chromatography (SiO₂, Petroleum ether/Ethyl acetate=1/0 to 0/1). 4 (650 mg, 2.39 mmol, 69.98% yield) was obtained as a yellow solid.

LCMS: RT=1.084 min, MS cal.: 272.1, [M+H]⁺=272.9

¹H NMR (400 MHz, CHCl3-d) δ=7.12 (d, J=1.6 Hz, 1H), 6.94 (d, J=1.6 Hz, 1H), 4.82 (dt, J=47.2, 4.0 Hz, 2H), 4.39 (dt, J=26.8, 4.0 Hz, 2H), 3.95 (s, 3H), 2.97 (s, 3H)

### General Procedure for Preparation of Intermediate 5:

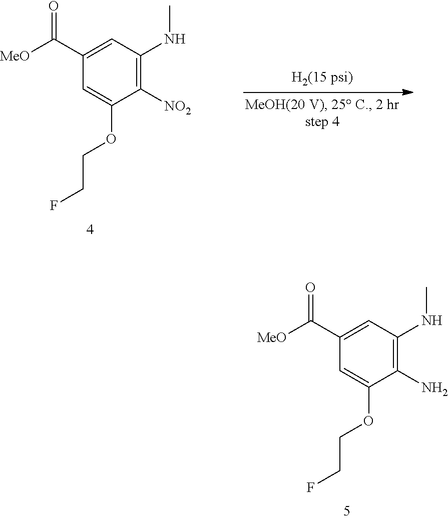 [SMILES: CNc1cc(C(=O)OC)cc(OCCF)c1[N+](=O)[O-] ; CNc1cc(C(=O)OC)cc(OCCF)c1N]

To a solution of 4 (350 mg, 1.29 mmol, 1 eq) in MeOH (7 mL) was added Pd/C (200 mg, 10% purity) at 25° C. Then reaction mixture was purged with H₂ for 3 times, the mixture was stirred at 25° C. for 2 hr under H₂ (15 Psi). LC-MS showed 4 was consumed completely and desired mass was detected. The reaction mixture was diluted with MeOH (30 ml), and then filtered through celite pad. The filter cake was rinsed with MeOH (5 ml×3), and the filtrate was concentrated to give the residue. The residue was purified by column chromatography (SiO₂, Petroleum ether/Ethyl acetate=1/0 to 5/1). 5 (300 mg, 1.24 mmol, 96.32% yield) was obtained as a yellow solid.

LCMS: RT=0.294, MS cal.: 242.3, [M+H]⁺=243.1

¹H NMR (400 MHz, CHCl3-d) δ=6.96 (d, J=1.6 Hz, 1H), 6.79 (d, J=1.6 Hz, 1H), 4.96-4.92 (br, 1H), 4.90 (br s, 2H), 4.80 (dt, J=48.0, 4.0 Hz, 2H), 4.24 (dt, J=30.0, 4.0 Hz, 2H), 3.75 (s, 3H), 2.75 (d, J=4.8 Hz, 3H)

### General Procedure for Preparation of Intermediate 6:

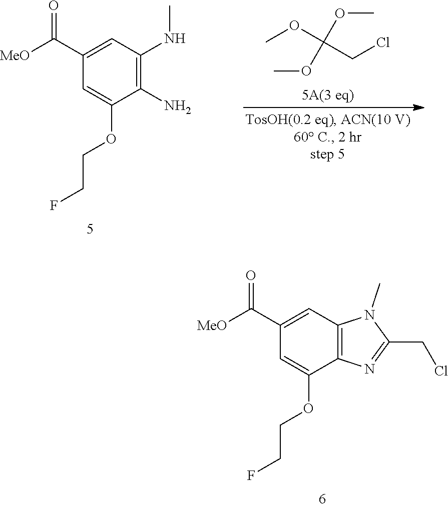

To a solution of 5 (200 mg, 825.61 μmol, 1 eq) in ACN (0.5 mL) was added 4-methylbenzenesulfonic acid (28.43 mg, 165.12 μmol, 0.2 eq) and 2-chloro-1,1,1-trimethoxy-ethane (382.90 mg, 2.48 mmol, 333.82 μL, 3 eq) at 25° C. The mixture was stirred at 60° C. for 2 hr. TLC showed 5 was consumed completely. The reaction mixture was diluted with H₂O 10 mL and extracted with EtOAc 8 mL. The combined organic layers were washed with brine 10 mL, dried over Na₂SO₄, filtered, and concentrated under reduced pressure to give 6 (200 mg, 665.09 μmol, 80.56% yield) as a pink solid.

LCMS: RT-0.443 min, MS cal.: 300.71, [M+H]⁺=301.0.

### General Procedure for Preparation of Intermediate 6:

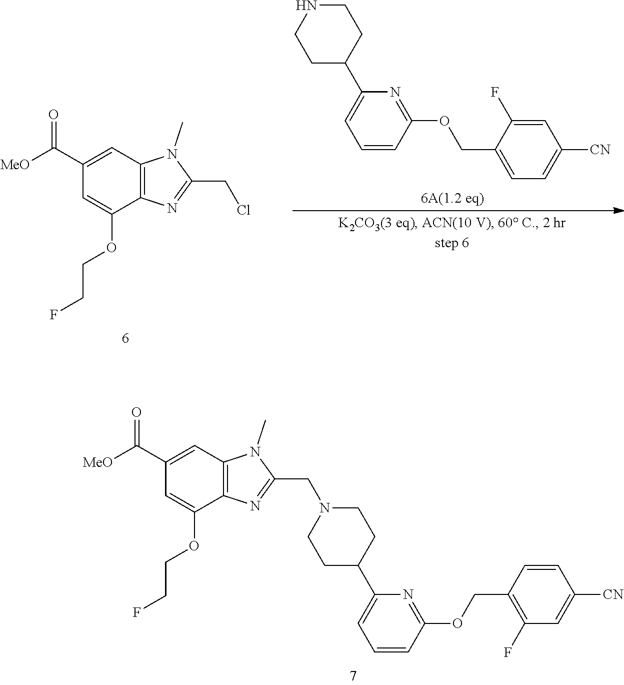 [SMILES: COC(=O)c1cc(OCCF)c2nc(CCl)n(C)c2c1 ; N#Cc1ccc(COc2cccc(C3CCNCC3)n2)c(F)c1 ; COC(=O)c1cc(OCCF)c2nc(CN3CCC(c4cccc(OCc5ccc(C#N)cc5F)n4)CC3)n(C)c2c1]

To a solution of 6 (100 mg, 332.54 μmol, 1 eq) in ACN (1 mL) was added K₂CO₃ (137.88 mg, 997.63 μmol, 3 eq) and 6A (124.25 mg, 399.05 μmol, 1.2 eq) at 25° C. The mixture was stirred at 60° C. for 2 hr. TLC showed 6 was consumed completely. The reaction mixture was diluted with H₂O 10 mL and extracted with EtOAc 9 mL. The combined organic layers were washed with brine 10 mL, dried over Na₂SO₄, filtered and concentrated under reduced pressure to give a residue. The residue was purified by column chromatography (SiO₂, Petroleum ether/Ethyl acetate=1/0 to 1/1). 7 (110 mg, 191.10 μmol, 57.47% yield) was obtained as a white solid.

LCMS: RT=1.462, MS cal.: 575.62, [M+H]⁺=576.0

¹H NMR (400 MHz, CHCl3-d) δ=7.90-7.86 (m, 1H), 7.85 (d, J=1.2 Hz, 1H), 7.69 (d, J=3.6 Hz, 2H), 7.64 (t, J=7.8 Hz, 1H), 7.29 (d, J=1.0 Hz, 1H), 6.87 (d, J=7.2 Hz, 1H), 6.71 (d, J=8.2 Hz, 1H), 5.45 (s, 2H), 4.88 (dt, J=48.0, 3.6 Hz, 2H), 4.55 (dt, J=30.0, 4.0 Hz, 2H), 3.93 (s, 3H), 3.88 (s, 3H), 3.82 (s, 2H), 2.91 (br d, J=11.2 Hz, 2H), 2.54 (s, 1H), 2.23-2.13 (m, 2H), 1.79-1.70 (m, 2H), 1.70-1.61 (m, 2H)

### General Procedure for Preparation of Compound 183 (8):

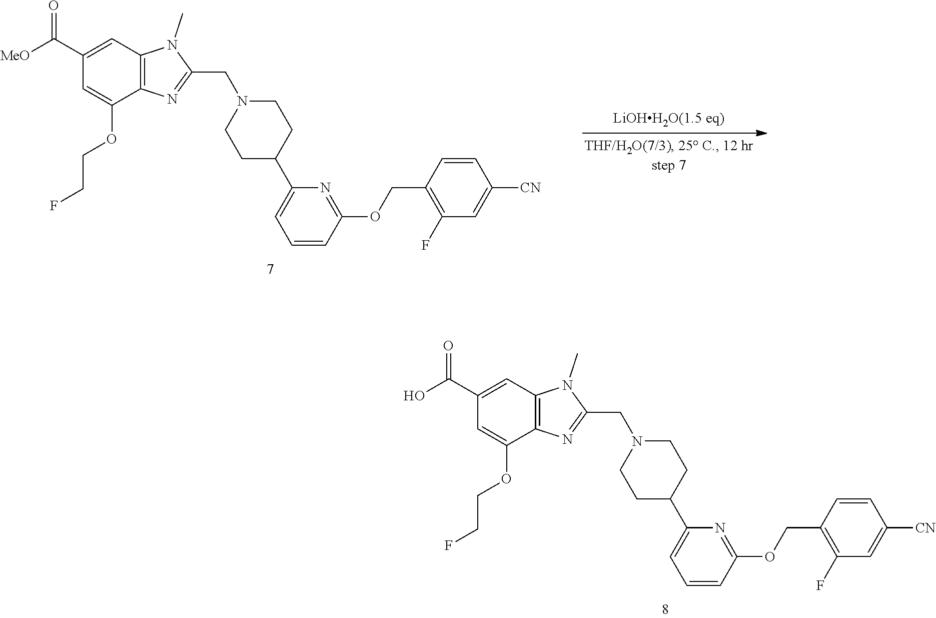 [SMILES: COC(=O)c1cc(OCCF)c2nc(CN3CCC(c4cccc(OCc5ccc(C#N)cc5F)n4)CC3)n(C)c2c1 ; Cn1c(CN2CCC(c3cccc(OCc4ccc(C#N)cc4F)n3)CC2)nc2c(OCCF)cc(C(=O)O)cc21]

To a solution of 7 (100 mg, 173.73 μmol, 1 eq) in THF (0.7 mL) was added LiOH·H₂O (10.94 mg, 260.60 μmol, 1.5 eq) and H₂O (0.3 mL) at 25° C. The mixture was stirred at 25° C. for 12 hr. TLC showed 7 was consumed completely. The reaction mixture was added with 0.5 M citric acid to adjust pH=7. The residue was purified by prep-HPLC (column: Waters Xbridge Prep OBD C18 150*40 mm*10 um; mobile phase: [H₂O (10 mM NH₄HCO₃)-ACN]; gradient: 25%-55% B over 8.0 min). 2-((4-(6-((4-Cyano-2-fluorobenzyl)oxy)pyridin-2-yl)piperidin-1-yl)methyl)-4-(2-fluoroethoxy)-1-methyl-1H-benzo[d]imidazole-6-carboxylic acid (29.18 mg, 51.96 μmol, 29.91% yield) was obtained as a white solid.

LCMS: RT=2.577, MS cal.: 561.59, [M+H]⁺=562.2

HPLC: RT=9.934 min, purity: 95.30%

¹H NMR (400 MHz, CHCl3-d) δ=7.88 (d, J=10.0 Hz, 1H), 7.81 (s, 1H), 7.69 (d, J=2.8 Hz, 2H), 7.64 (t, J=7.6 Hz, 1H), 7.29 (s, 1H), 6.88 (d, J=7.2 Hz, 1H), 6.71 (d, J=8.2 Hz, 1H), 5.45 (s, 2H), 4.88 (dt, J=48.0, 4.0 Hz, 2H), 4.53 (dt, J=26.8, 3.6 Hz, 2H), 3.91 (s, 3H), 3.81 (s, 2H), 2.91 (br d, J=10.8 Hz, 2H), 2.62-2.54 (m, 1H), 2.18 (br t, J=10.8 Hz, 2H), 1.79-1.71 (m, 2H), 1.70-1.59 (m, 2H)

### Example A182—Preparation of Compound 184. 2-((4-(4-((4-Cyano-2-fluorobenzyl)oxy)pyrimidin-2-yl)piperidin-1-yl)methyl)-4-(difluoromethoxy)-1-methyl-1H-benzo[d]imidazole-6-carboxylic acid (Compound 184) (7)

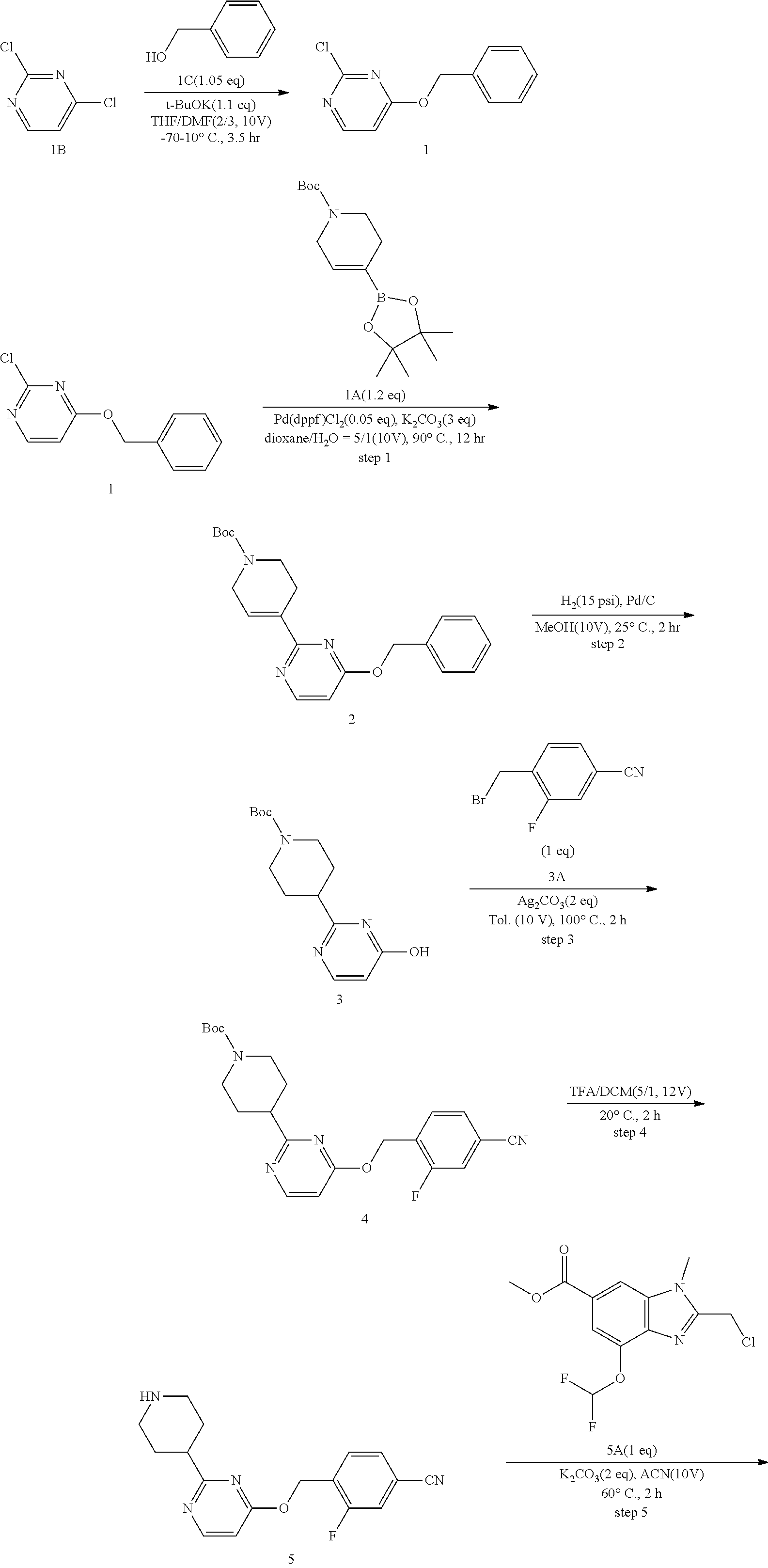

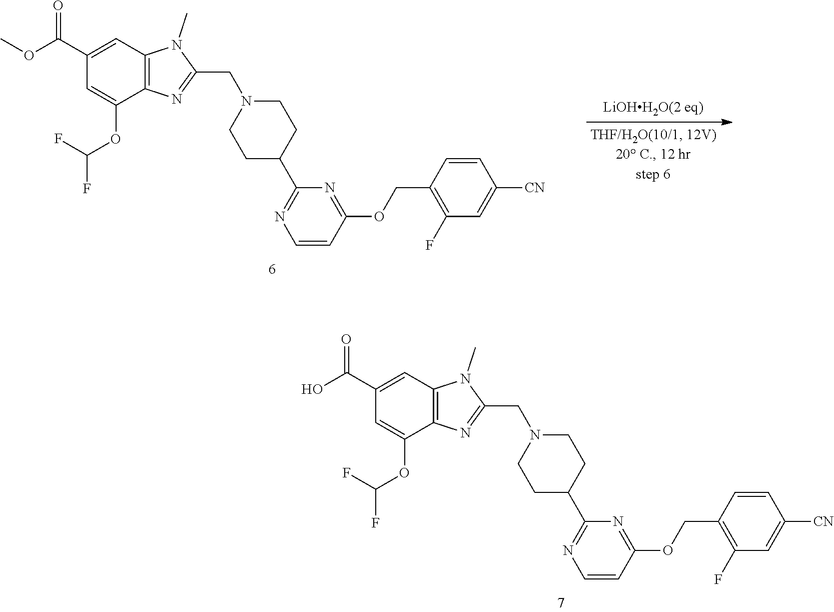 [SMILES: COC(=O)c1cc(OC(F)F)c2nc(CN3CCC(c4nccc(OCc5ccc(C#N)cc5F)n4)CC3)n(C)c2c1 ; Cn1c(CN2CCC(c3nccc(OCc4ccc(C#N)cc4F)n3)CC2)nc2c(OC(F)F)cc(C(=O)O)cc21]

### General Procedure for Preparation of Intermediate 1:

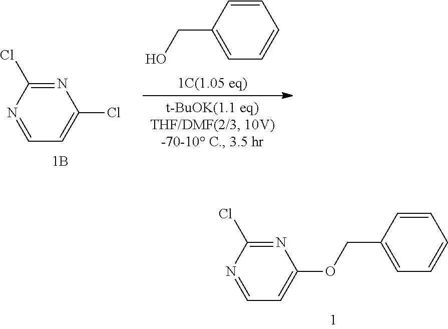 [SMILES: Clc1ccnc(Cl)n1 ; OCc1ccccc1 ; Clc1nccc(OCc2ccccc2)n1]

THF (400 mL) was charged to the 1 L reactor A. 1C (76.22 g, 704.80 mmol, 73.28 mL, 1.05 eq) to the 1 L reaction reactor. t-BuOK (82.85 g, 738.36 mmol, 1.1 eq) was added to the reactor at 20° C. (obvious exotherm: ˜ 10° C.) and stirred until it was dissolved completed.

DMF (600 mL) was charged to another 2 L reactor B. 1B (100 g, 671.24 mmol, 1 eq) to the 2 L reaction reactor. The mixture in reactor B was cooled to −65° C. The solution in reactor A was added to reactor B at −70° C.˜−65° C. dropwise slowly for 1.5 hour under N₂ atmosphere. ((obvious exotherm: <−55° C.). The reaction mixture was warmed to 10° C. slowly for 1.5 hours and stirred for 0.5 hr. HPLC showed 0.3% 1C and 85.9% 1. The reaction mixture was poured into H₂O (3 L) slowly at 0° C. and the solid formed. The mixture was stirred for 15 min and filtered. The solid was collected, dissolved in DCM (500 mL) and washed with brine (200 mL), dried over Na₂SO₄, filtered and concentrated under reduced pressure to give crude product (150 g). The crude product was stirred in MTBE (100 mL) and stirred for 5 min at 20° C., n-hexane (500 mL) was added to the mixture, stirred for 10 min, filtered and the filter cake was collected. The filter cake was added MTBE (50 mL) and stirred for 5 min, n-hexane (250 mL) was added to the mixture, stirred for 5 min, filtered and the filter cake was collected, dried under vacuum for 20 min to afford the product. 1 (92 g, 416.94 mmol, 62.12% yield) was obtained as a white solid.

HPLC: Product RT=2.977 min

¹H NMR (400 MHz, DMSO-d₆) δ=8.49 (d, J=6.0 Hz, 1H), 7.51-7.46 (m, 2H), 7.43-7.32 (m, 3H), 7.05 (d, J=6.0 Hz, 1H), 5.41 (s, 2H)

### General Procedure for Preparation of Intermediate 2:

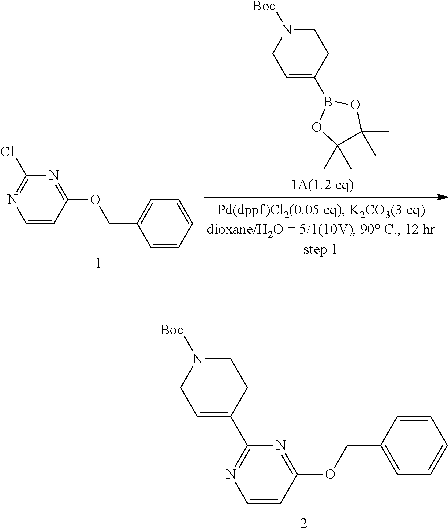 [SMILES: Clc1nccc(OCc2ccccc2)n1 ; CC(C)(C)OC(=O)N1CC=C(B2OC(C)(C)C(C)(C)O2)CC1 ; CC(C)(C)OC(=O)N1CC=C(c2nccc(OCc3ccccc3)n2)CC1]

A solution of 1 (2 g, 9.06 mmol, 1 eq) and 1A (3.36 g, 10.88 mmol, 1.2 eq) in dioxane (16.6 mL) and H₂O (3.3 mL) was added Pd(dppf)Cl₂ (331.61 mg, 453.20 μmol, 0.05 eq) and K₂CO₃ (3.76 g, 27.19 mmol, 3 eq) at 20° C. under N₂. The mixture was stirred at 90° C. for 12 hr. TLC (Petroleum ether:Ethyl acetate=3:1, Rf=0.5) indicated Reactant 1 was consumed completely and one new spot formed. The reaction mixture was diluted with H₂O (30 mL) and filtered through celite pad. The filter cake was rinsed with EtOAc (10 ml*2) and the filtrate was extracted with EtOAc (15 mL*3). The combined organic layers were washed with brine (20 mL*2), dried over Na₂SO₄, filtered, and concentrated under reduced pressure to give a residue. The residue was purified by column chromatography (SiO₂, Petroleum ether/Ethyl acetate=19/1 to 5/1). 2 (2.6 g, 7.08 mmol, 78% yield) was obtained as a colorless oil.

LCMS: RT=1.514 min, MS cal.: 367.4, [M+H]⁺=368.0

¹H NMR (400 MHz, CHCl3-d) δ=8.42 (d, J=6 Hz, 1H), 7.48-7.32 (m, 5H), 7.20 (s, J=2.0 Hz, 1H), 6.62 (d, J=5.6 Hz, 1H), 5.47 (s, 2H), 4.19 (s, 2H), 3.64 (t, J=5.6 Hz, 2H), 2.72 (s, 2H), 1.50 (s, 9H)

### General Procedure for Preparation of Intermediate 3:

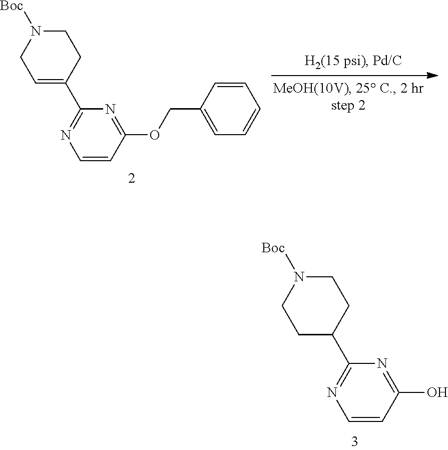 [SMILES: CC(C)(C)OC(=O)N1CC=C(c2nccc(OCc3ccccc3)n2)CC1 ; CC(C)(C)OC(=O)N1CCC(c2nccc(O)n2)CC1]

Pd/C (1.3 g) was charged to the round bottom flask, then MeOH (26 mL) was added at 25° C. under Ar, then 2 (2.6 g, 7.08 mmol, 1 eq) was added to the reaction mixture under N₂ atmosphere. After the addition, the mixture was degassed and purged with H₂ for 3 times, the mixture was stirred at 25° C. for 2 hr under H₂ (15 psi). TLC (Petroleum ether:Ethyl acetate=1:1, Rf=0.1) indicated Compound 2 was consumed completely and one new spot formed. The reaction mixture was diluted with MeOH (5 mL) and filtered through celite pad. The filter cake was rinsed with MeOH (10 mL*2) and the filtrate was concentrated under reduced pressure to give a crude product. The residue was purified by column chromatography (SiO₂, Petroleum ether/Ethyl acetate=10/1 to 1/1). 3 (1.3 g, 4.65 mmol, 66% yield) was obtained as a white solid.

LCMS: RT=0.851 min, MS cal.: 321.8, [M+H]⁺=280.0

¹H NMR (400 MHz, CHCl3-d) δ=13.33-12.53 (br, 1H), 8.00 (d, J=6.8 Hz, 1H), 6.35 (d, J=6.8 Hz, 1H), 4.35-4.10 (m, 2H), 2.98-2.68 (m, 3H), 1.99-1.89 (m, 2H), 1.88-1.74 (m, 2H), 1.48 (s, 9H)

### General Procedure for Preparation of Intermediate 4:

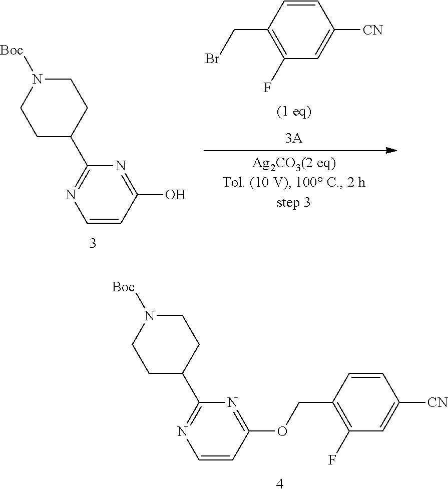 [SMILES: CC(C)(C)OC(=O)N1CCC(c2nccc(O)n2)CC1 ; N#Cc1ccc(CBr)c(F)c1 ; CC(C)(C)OC(=O)N1CCC(c2nccc(OCc3ccc(C#N)cc3F)n2)CC1]

A solution of 3 (500 mg, 1.79 mmol, 1 eq) and 3A (383.12 mg, 1.79 mmol, 1 eq) in Toluene (5 mL) was added Ag₂CO₃ (987.15 mg, 3.58 mmol, 162.44 μL, 2 eq) at 20° C. The mixture was stirred at 100° C. for 2 hr. LC-MS (Rt=1.367) showed Compound 3 was consumed completely and desired mass was detected. The reaction mixture was diluted with H₂O (20 mL) and filtered through celite pad. The filter cake was rinsed with EtOAc (10 mL*2) and the filtrate was extracted with EtOAc (10 mL*2). The combined organic layers were washed with brine (10 mL*2), filtered, and concentrated under reduced pressure to give a crude product. The residue was purified by column chromatography (SiO₂, Petroleum ether/Ethyl acetate=10/1 to 0/1). 4 (530 mg, 1.28 mmol, 72% yield) was obtained as a white solid.

LCMS: RT=1.367 min, MS cal.: 412.2, [M+H]⁺=413.0

¹H NMR (400 MHz, CHCl3-d) δ=8.43 (d, J=5.6 Hz, 1H), 7.65-7.56 (t, J=7.6 Hz, 1H), 7.52-7.46 (dd, J=9.2 Hz, 1H), 7.42 (dd, J=9.2, 1.2 Hz, 1H), 6.66 (d, J=5.6 Hz, 1H), 5.55 (s, 2H), 4.30-4.11 (s, 2H), 3.01-2.78 (m, 3H), 1.96 (d, J=11.6 Hz, 2H), 1.77 (m, 2H), 1.49 (s, 9H)

### General Procedure for Preparation of Intermediate 5:

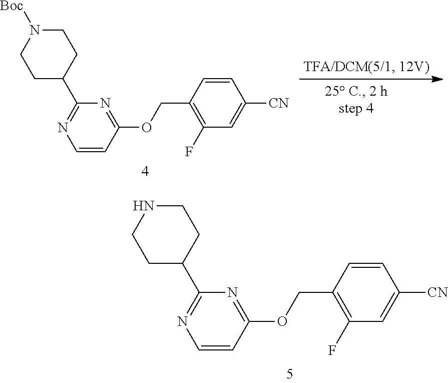 [SMILES: CC(C)(C)OC(=O)N1CCC(c2nccc(OCc3ccc(C#N)cc3F)n2)CC1 ; N#Cc1ccc(COc2ccnc(C3CCNCC3)n2)c(F)c1]

A solution of 4 (250 mg, 606.12 μmol, 1 eq) in DCM (2.5 mL) was added TFA (0.5 mL) at 25° C. The mixture was stirred at 25° C. for 2 hr. TLC (Petroleum ether:Ethyl acetate-0:1, Rf=0.1) indicated Compound 4 was consumed completely and one new spot formed. The reaction mixture was added saturated sodium carbonate aqueous solution to adjust pH-8, diluted with H₂O (10 mL) and extracted with DCM (3 mL*3). The combined organic layers were washed with brine (5 mL*2), dried over Na₂SO₄, filtered, and concentrated under reduced pressure to give a crude product. 5 (180 mg, 576.29 μmol, 95% yield) was obtained as a white solid.

LCMS: RT=0.842 min, MS cal.: 312.3, [M+H]⁺=313.0

¹H NMR (400 MHz, DMSO-d6) δ=8.48 (d, J=6 Hz, 1H), 7.92 (d, J=10 Hz, 1H), 7.74 (d, J=3.6 Hz, 2H), 6.84 (d, J=5.6 Hz, 1H), 5.54 (s, 2H), 3.01 (dt, J=12 Hz, 2H), 2.83-2.73 (m, 1H), 2.59 (td, J=12, 2.4 Hz, 2H), 1.81 (dd, J=10.4 Hz, 2H), 1.68-1.54 (m, 2H)

### General Procedure for Preparation of Intermediate 6:

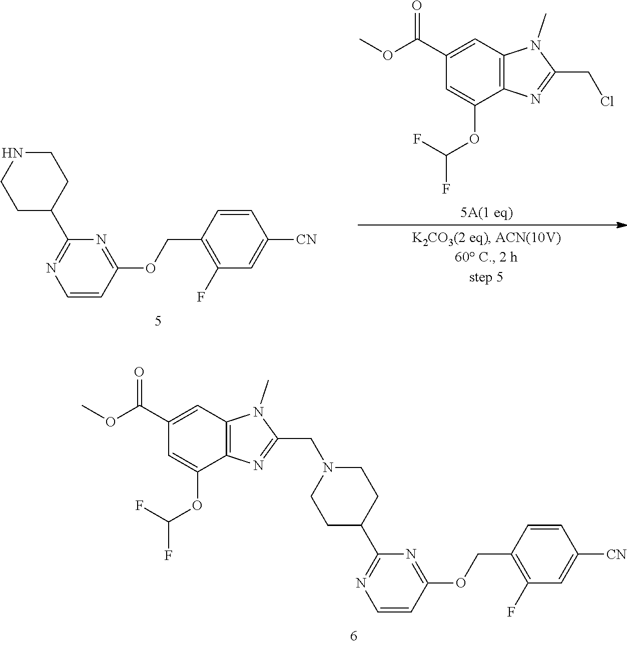 [SMILES: N#Cc1ccc(COc2ccnc(C3CCNCC3)n2)c(F)c1 ; COC(=O)c1cc(OC(F)F)c2nc(CCl)n(C)c2c1 ; COC(=O)c1cc(OC(F)F)c2nc(CN3CCC(c4nccc(OCc5ccc(C#N)cc5F)n4)CC3)n(C)c2c1]

To a solution of 5 (150 mg, 480.24 μmol, 1 eq) and 5A (146.32 mg, 480.24 μmol, 1 eq) in ACN (1.5 mL) was added K₂CO₃ (132.74 mg, 960.49 μmol, 2 eq) at 20° C. The mixture was stirred at 60° C. for 2 hr. LC-MS (Rt=1.382) showed Compound 4 was consumed completely and desired mass was detected. The reaction mixture was added H₂O (10 mL), and then extracted with

EtOAc (5 mL*3). The combined organic layers were washed with brine (10 mL*2), dried over Na₂SO₄, filtered, and concentrated under reduced pressure to give a crude product. The residue was purified by column chromatography (SiO₂, Petroleum ether/Ethyl acetate=10/1 to 0/1). 6 (230 mg, 396.17 μmol, 82% yield) was obtained as a white solid.

LCMS: RT=1.382 min, MS cal.: 580.2, [M+H]⁺=581.1

¹H NMR (400 MHz, CHCl3-d) δ=8.42 (d, J=5.6 Hz, 1H), 7.99 (s, 1H), 7.72 (s, 1H), 7.65-7.57 (t, J=7.6 Hz, 1H), 7.47 (d, J=8.0 Hz, 1H), 7.40 (d, J=9.2 Hz, 1H), 7.31 (t, J=74.4, 1H), 6.65 (d, J=5.6 Hz, 1H), 5.54 (s, 2H), 4.00 (s, 3H), 3.97 (s, 3H), 3.91 (s, 2H), 2.97 (s, 2H), 2.88-2.74 (s, 1H), 2.41-2.25 (s, 2H), 1.99 (s, 2H), 1.94-1.80 (s, 2H)

### General Procedure for Preparation of Compound 184 (7):

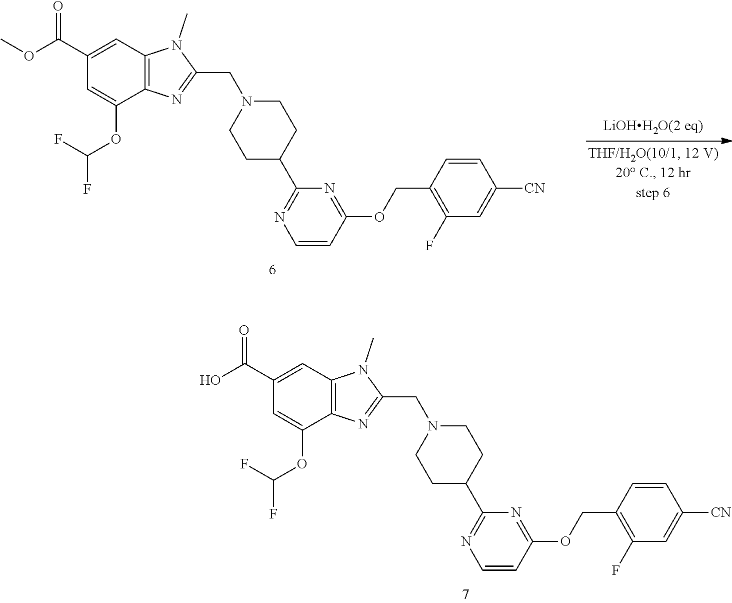 [SMILES: COC(=O)c1cc(OC(F)F)c2nc(CN3CCC(c4nccc(OCc5ccc(C#N)cc5F)n4)CC3)n(C)c2c1 ; Cn1c(CN2CCC(c3nccc(OCc4ccc(C#N)cc4F)n3)CC2)nc2c(OC(F)F)cc(C(=O)O)cc21]

A solution of 6 (180 mg, 310.05 μmol, 1 eq) in THF (2 mL) was added LiOH·H₂O (26.02 mg, 620.09 μmol, 2 eq) in H₂O (0.2 mL) at 20° C. The mixture was stirred at 20° C. for 12 hr. LC-MS (Rt=1.366) showed Compound 6 was consumed completely and desired mass was detected. The reaction mixture was quenched with 1 N HCl to adjust pH=8. The residue was purified by prep-HPLC (column: Waters Xbridge Prep OBD C18 150*40 mm*10 um; mobile phase: [H₂O (10 mM NH₄HCO₃)-ACN]; gradient: 15%-55% B over 8.0 min). 2-((4-(4-((4-Cyano-2-fluorobenzyl)oxy)pyrimidin-2-yl)piperidin-1-yl)methyl)-4-(difluoromethoxy)-1-methyl-1H-benzo[d]imidazole-6-carboxylic acid (29.77 mg, 52.55 μmol, 17% yield) was obtained as a white solid.

LCMS: RT=1.366 min, MS cal.: 566.5, [M+H]⁺=567.1

HPLC: RT=9.452 purity: 99.73%

¹H NMR (400 MHz, MeOH-d4) δ=8.48 (d, J=5.6 Hz, 1H), 8.06 (d, J=1.2 Hz, 1H), 7.90 (d, J=10.4 Hz, 1H), 7.76-7.69 (m, 2H), 7.63 (t, J=74.4 Hz, 1H), 7.52 (s, 1H), 6.84 (d, J=6 Hz, 1H), 5.53 (s, 2H), 3.96 (s, 3H), 3.85 (s, 2H), 2.90 (d, J=11.2 Hz, 2H), 2.77-2.65 (m, 1H), 2.23 (t, J=10.0 Hz, 2H), 1.88 (d, J=10.8 Hz, 2H), 1.80-1.67 (m, 2H)

### Example A183—Preparation of Compound 139. 4-(Difluoromethoxy)-2-((4-(3-fluoro-2-((2-fluoro-4-methylbenzyl)oxy)phenyl)piperidin-1-yl)methyl)-1-methyl-1H-benzo[d]imidazole-6-carboxylic acid (Compound 139) (5)

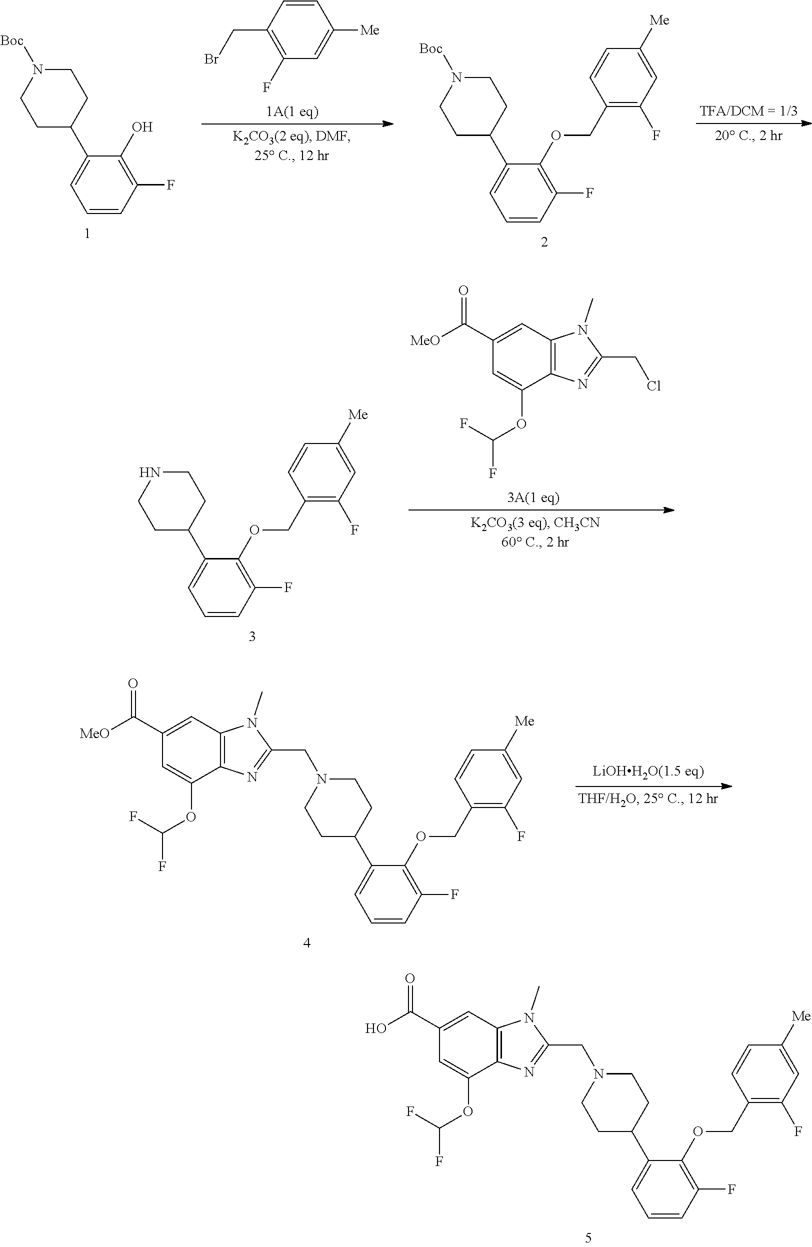

### General Procedure for Preparation of Intermediate 2:

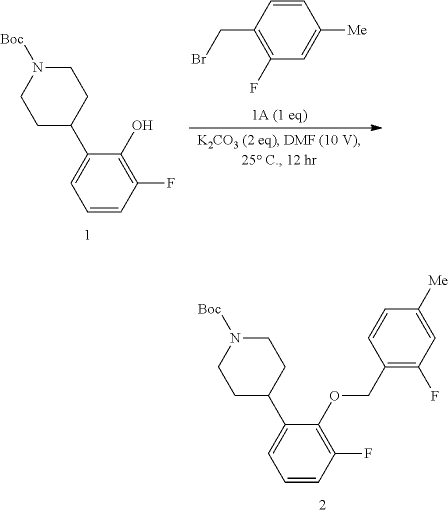 [SMILES: CC(C)(C)OC(=O)N1CCC(c2cccc(F)c2O)CC1 ; Cc1ccc(CBr)c(F)c1 ; Cc1ccc(COc2c(F)cccc2C2CCN(C(=O)OC(C)(C)C)CC2)c(F)c1]

To a solution of 1 (150 mg, 507.87 μmol, 1 eq) in DMF (2 mL) was added K₂CO₃ (140.38 mg, 1.02 mmol, 2 eq) and 1A (103.12 mg, 507.87 μmol, 1 eq). The mixture was stirred at 25° C. for 12 hr. TLC (Petroleum ether:Ethyl acetate=3:1, Rf=0.67) indicated 1 was consumed completely and one new spot formed. The reaction mixture was diluted with H₂O 10 mL and extracted with EtOAc 30 mL (10 mL*3). The combined organic layers were washed with brine 10 mL, dried over Na₂SO₄, filtered and concentrated under reduced pressure to give a residue. The residue was purified by column chromatography (SiO₂, Petroleum ether/Ethyl acetate=20/1 to 1/1). 2 (150 mg, 359.29 μmol, 70.74% yield) was obtained as colorless oil.

LCMS: RT=2.428 min, MS cal.: 417.21, [M−55]⁺=362.0

¹H NMR (400 MHz, CHCl3-d) δ=7.23-7.20 (m, 1H), 6.97-6.78 (m, 5H), 5.03 (s, 2H), 4.19-3.97 (m, 2H), 3.03-2.88 (m, 1H), 2.68-2.53 (m, 2H), 2.28 (s, 3H), 1.46-1.31 (m, 13H)

### General Procedure for Preparation of Intermediate 3:

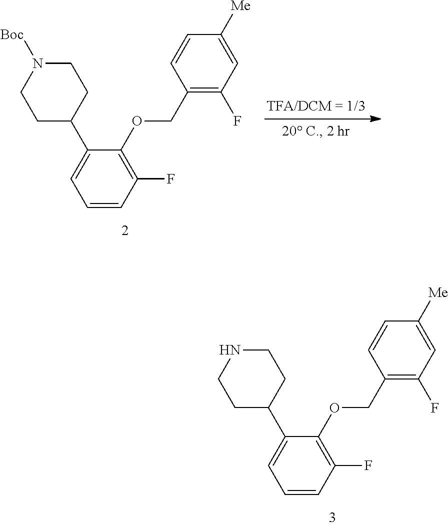 [SMILES: Cc1ccc(COc2c(F)cccc2C2CCN(C(=O)OC(C)(C)C)CC2)c(F)c1 ; Cc1ccc(COc2c(F)cccc2C2CCNCC2)c(F)c1]

To a solution of 2 (130 mg, 311.39 μmol, 1 eq) in DCM (1.5 mL) was added TFA (767.50 mg, 6.73 mmol, 0.5 mL, 21.62 eq). The mixture was stirred at 20° C. for 2 hr. LC-MS showed 2 was consumed completely and desired mass was detected. The reaction mixture was concentrated under reduced pressure to remove solvent. 3 (130 mg, 301.35 μmol, 96.78% yield, TFA) was obtained as colorless oil.

LCMS: RT=1.113 min, MS cal.: 317.1, [M+H]⁺=318.0

### General Procedure for Preparation of Intermediate 4:

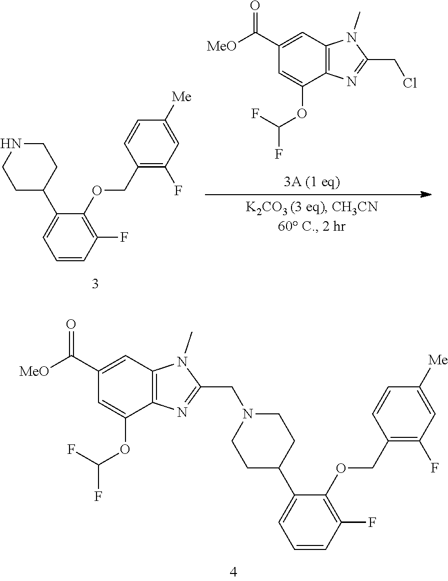 [SMILES: Cc1ccc(COc2c(F)cccc2C2CCNCC2)c(F)c1 ; COC(=O)c1cc(OC(F)F)c2nc(CCl)n(C)c2c1 ; COC(=O)c1cc(OC(F)F)c2nc(CN3CCC(c4cccc(F)c4OCc4ccc(C)cc4F)CC3)n(C)c2c1]

To a solution of 3 (130 mg, 301.35 μmol, 1 eq, TFA) in CH₃CN (2 mL) was added K₂CO₃ (124.94 mg, 904.04 μmol, 3 eq) and 3A (91.81 mg, 301.35 μmol, 1 eq). The mixture was stirred at 60° C. for 2 hr. LC-MS showed 3 was consumed completely and desired mass was detected. The residue was diluted with H₂O 15 mL and extracted with EtOAc 45 mL (15 mL*3). The combined organic layers were washed with brine 15 mL, dried over Na₂SO₄, filtered, and concentrated under reduced pressure to give a residue. The residue was purified by column chromatography (SiO₂, Petroleum ether:Ethyl acetate=10/1 to 0/1) (Rf=0.57). 4 (120 mg, 204.92 μmol, 68.00% yield) was obtained as a white solid.

LCMS: RT=1.683 min, MS cal.: 585.2, [M+H]⁺=586.1

¹H NMR (400 MHz, MeOH-d4) δ=8.14 (d, J=1.2 Hz, 1H), 7.69 (s, 1H), 7.28 (t, J=7.8 Hz, 1H), 7.28 (t, J=74 Hz, 1H) 7.05-6.92 (m, 5H), 5.10 (s, 2H), 4.01 (s, 3H), 3.98 (s, 3H), 3.89 (s, 2H), 3.07-2.80 (m, 3H), 2.36 (s, 3H), 2.19 (br t, J=10.8 Hz, 2H), 1.70-1.55 (m, 2H), 1.48 (br d, J=11.6 Hz, 2H)

### General Procedure for Preparation of Compound 139 (5):

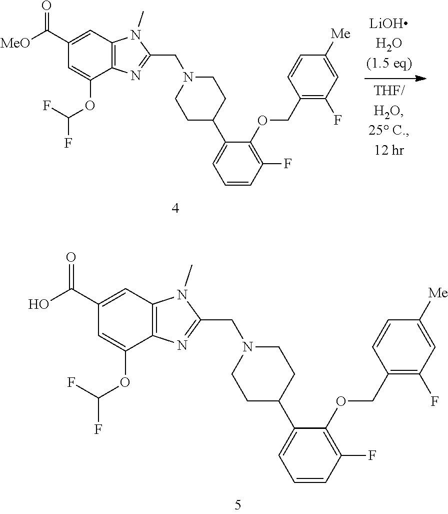 [SMILES: COC(=O)c1cc(OC(F)F)c2nc(CN3CCC(c4cccc(F)c4OCc4ccc(C)cc4F)CC3)n(C)c2c1 ; Cc1ccc(COc2c(F)cccc2C2CCN(Cc3nc4c(OC(F)F)cc(C(=O)O)cc4n3C)CC2)c(F)c1]

To a solution of 4 (80 mg, 136.61 μmol, 1 eq) in THF (0.7 mL) was added LiOH·H₂O (8.60 mg, 204.92 μmol, 1.5 eq) in H₂O (0.3 mL). The mixture was stirred at 25° C. for 12 hr. LC-MS showed 4 was consumed completely and desired mass was detected. The reaction was added citric acid (0.5 M) to pH=8, then the mixture was purified directly. The residue was purified by prep-HPLC (neutral condition; column: Waters Xbridge BEH C18 100*30 mm*10 um; mobile phase: [H₂O (10 mM NH₄HCO₃)-ACN]; gradient: 30%-60% B over 8.0 min). 4-(Difluoromethoxy)-2-((4-(3-fluoro-2-((2-fluoro-4-methylbenzyl)oxy)phenyl)piperidin-1-yl)methyl)-1-methyl-1H-benzo[d]imidazole-6-carboxylic acid (29.33 mg, 51.15 μmol, 37.44% yield, 99.67% purity) was obtained as a white solid.

LCMS: RT=2.448 min, MS cal.: 571.2, [M+H]⁺=566.2

HPLC: RT=12.313 min, purity: 98.35%

¹H NMR (400 MHz, DMSO-d6) δ=8.06 (d, J=1.2 Hz, 1H), 7.63 (t, J=74.4 Hz, 1H), 7.52 (s, 1H), 7.31 (t, J=8.0 Hz, 1H), 7.14-6.99 (m, 5H), 5.03 (s, 2H), 3.93 (s, 3H), 3.82 (s, 2H), 2.88 (br d, J=11.2 Hz, 2H), 2.77 (ddd, J=3.2, 8.8, 12.0 Hz, 1H), 2.31 (s, 3H), 2.07 (br t, J=10.8 Hz, 2H), 1.52 (dq, J=2.8, 12.0 Hz, 2H), 1.38 (br d, J=11.2 Hz, 2H)

### Example A184—Preparation of Compound 140. 2-((4-(2-((4-Chloro-2-fluorobenzyl)oxy)-3-fluorophenyl)piperidin-1-yl)methyl)-4-(difluoromethoxy)-1-methyl-1H-benzo[d]imidazole-6-carboxylic acid (Compound 140) (5)

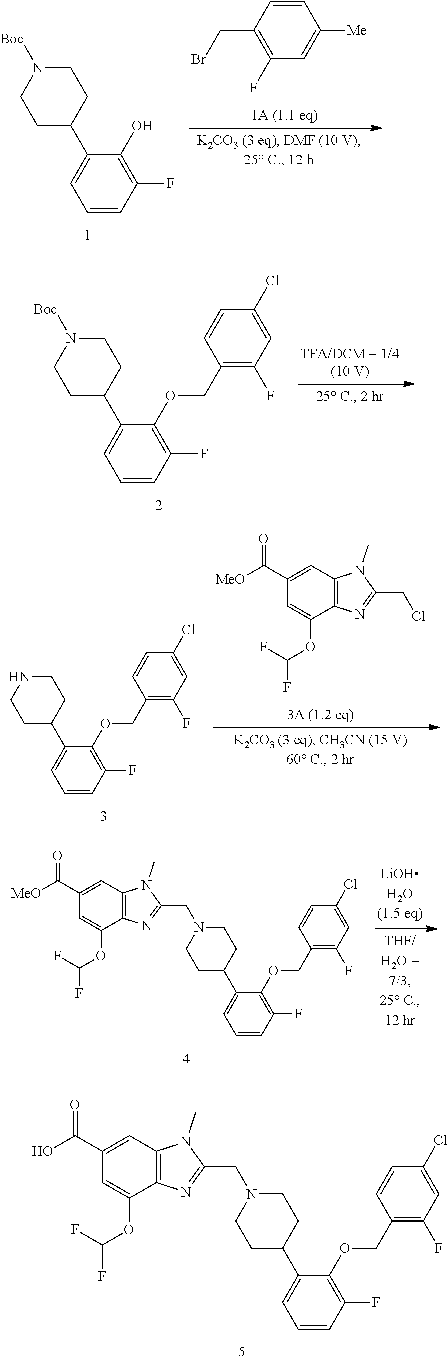

### General Procedure for Preparation of Intermediate 2:

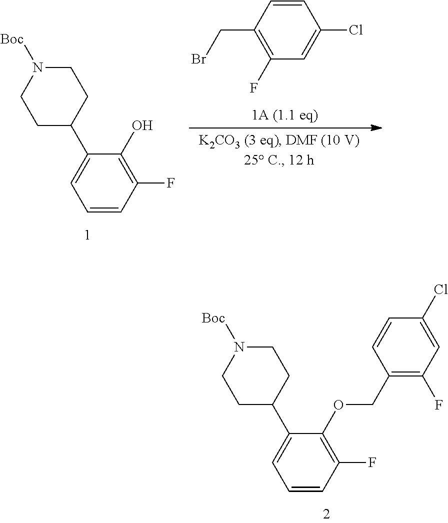 [SMILES: CC(C)(C)OC(=O)N1CCC(c2cccc(F)c2O)CC1 ; Fc1cc(Cl)ccc1CBr ; CC(C)(C)OC(=O)N1CCC(c2cccc(F)c2OCc2ccc(Cl)cc2F)CC1]

A mixture of 1 (200 mg, 677.17 μmol, 1 eq), 1A (166.46 mg, 744.88 μmol, 1.1 eq), K₂CO₃ (280.76 mg, 2.03 mmol, 3 eq) in DMF (2 mL) was degassed and purged with N₂ for 3 times, and then the mixture was stirred at 25° C. for 12 hr under N₂ atmosphere. TLC (PE:EA=3:1, Rf=0.59) indicated 1 was consumed completely one new spot formed. The reaction mixture was diluted with H₂O 15 mL and extracted with EtOAc 45 mL (15 mL*3). The combined organic layers were washed with brine 15 mL, dried over Na₂SO₄, filtered, and concentrated under reduced pressure to give a residue. The residue was purified by column chromatography (SiO₂, Petroleum ether/Ethyl acetate=10/1 to 3/1). 2 (100 mg, 228.36 μmol, 67.45% yield) was obtained as colorless oil.

LCMS: RT=1.726 min, MS cal.: 437.1, [M−55]⁺=383.9

¹H NMR (400 MHz, CDCl₃) δ=7.47-7.37 (m, 1H), 7.20-7.11 (m, 2H), 7.06-6.95 (m, 2H), 6.92 (d, J=6.8 Hz, 1H), 5.13-5.09 (m, 2H), 4.24-4.16 (m, 2H), 3.02 (tt, J=3.6, 11.2 Hz, 1H), 2.78-2.66 (m, 2H), 1.63-1.50 (m, 4H), 1.48 (s, 10H)

### General Procedure for Preparation of Intermediate 3:

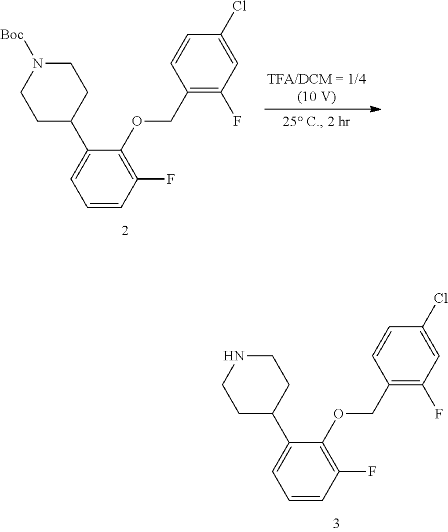 [SMILES: CC(C)(C)OC(=O)N1CCC(c2cccc(F)c2OCc2ccc(Cl)cc2F)CC1 ; Fc1cc(Cl)ccc1COc1c(F)cccc1C1CCNCC1]

To a solution of 2 (200 mg, 456.72 μmol, 1 eq) in DCM (2 mL) was added TFA (767.50 mg, 6.73 mmol, 0.5 mL, 14.74 eq). The mixture was stirred at 25° C. for 2 hr. TLC (Petroleum ether:Ethyl acetate=3:1, Rf=0.08) indicated 2 was consumed completely and one new spot formed. The reaction was clean according to TLC. The reaction mixture was concentrated under reduced pressure to remove solvent. 3 (200 mg, 442.66 μmol, 96.92% yield, TFA) was obtained as a white oil.

LCMS: RT=1.132 min MS cal: 337.1 [M+H]⁺=337.9

¹H NMR (400 MHz, CDCl₃) δ=11.30 (br s, 2H), 7.43-7.35 (m, 1H), 7.21-7.13 (m, 2H), 7.11-7.00 (m, 2H), 6.97-6.90 (m, 1H), 5.13 (s, 2H), 3.57 (br d, J=12.2 Hz, 2H), 3.28-3.14 (m, 1H), 3.01 (q, J=11.2 Hz, 2H), 2.00-1.79 (m, 4H)

### General Procedure for Preparation of Intermediate 4:

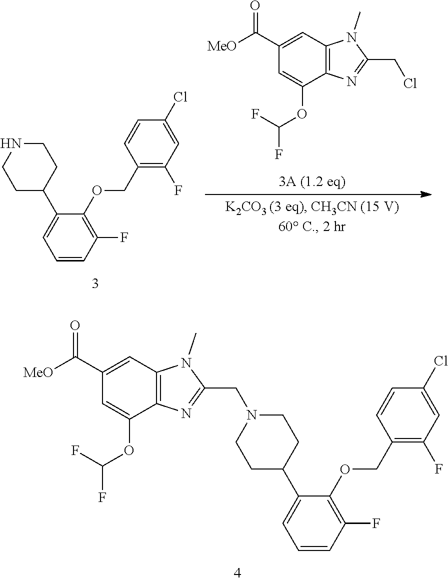 [SMILES: Fc1cc(Cl)ccc1COc1c(F)cccc1C1CCNCC1 ; COC(=O)c1cc(OC(F)F)c2nc(CCl)n(C)c2c1 ; COC(=O)c1cc(OC(F)F)c2nc(CN3CCC(c4cccc(F)c4OCc4ccc(Cl)cc4F)CC3)n(C)c2c1]

A mixture of 3 (200 mg, 592.08 μmol, 1 eq), 3A (216.47 mg, 710.50 μmol, 1.2 eq), K₂CO₃ (245.49 mg, 1.78 mmol, 3 eq) in CH₃CN (3 mL) was degassed and purged with N₂ for 3 times, and then the mixture was stirred at 60° C. for 2 hr under N₂ atmosphere. TLC (Petroleum ether:Ethyl acetate=3:1, Rf=0.67) indicated 3 was consumed completely and one new spot formed. The reaction was clean according to TLC. The reaction mixture diluted with H₂O 30 mL and extracted with 45 mL (15 mL*3). The combined organic layers were washed with brine 15 mL, dried over Na₂SO₄, filtered and concentrated under reduced pressure to give a residue. The residue was purified by column chromatography (SiO₂, Petroleum ether/Ethyl acetate=10/1 to 3/1). 4 (200 mg, 330.03 μmol, 55.74% yield) was obtained as a white solid.

LCMS: RT=1.691 min, MS cal.: 605.2, [M+H]⁺=606.0

### General Procedure for Preparation of Compound 140 (5):

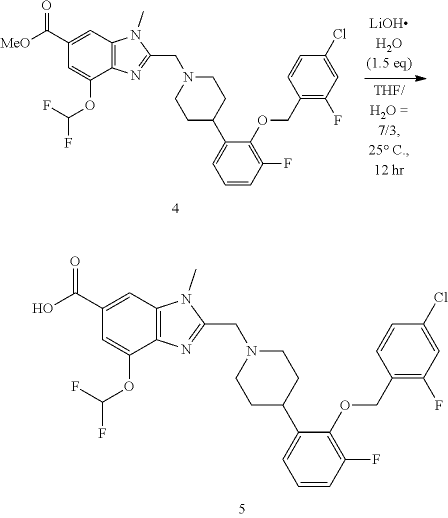 [SMILES: COC(=O)c1cc(OC(F)F)c2nc(CN3CCC(c4cccc(F)c4OCc4ccc(Cl)cc4F)CC3)n(C)c2c1 ; Cn1c(CN2CCC(c3cccc(F)c3OCc3ccc(Cl)cc3F)CC2)nc2c(OC(F)F)cc(C(=O)O)cc21]

To a solution of 4 (160 mg, 264.02 μmol, 1 eq) in THF (1.4 mL) was added LiOH·H₂O (16.62 mg, 396.04 μmol, 1.5 eq) in H₂O (0.6 mL) was degassed and purged with N₂ for 3 times, and then the mixture was stirred at 25° C. for 12 hr under N₂ atmosphere. LC-MS (product, RT=1.707 min) showed 4 was consumed completely and desired mass was detected. The residue was purified by prep-HPLC (neutral condition, column: Waters Xbridge BEH C18 100*30 mm*10 um; mobile phase: [H₂O (10 mM NH₄HCO₃)-ACN]; gradient: 35%-65% B over 8.0 min). 2-((4-(2-((4-Chloro-2-fluorobenzyl)oxy)-3-fluorophenyl)piperidin-1-yl)methyl)-4-(difluoromethoxy)-1-methyl-1H-benzo[d]imidazole-6-carboxylic acid (31.22 mg, 52.41 μmol, 19.85% yield, 99.38% purity) was obtained as a white solid.

LCMS: RT=2.477 min, MS cal.: 591.2, [M+H]⁺=592.2

HPLC: RT=12.472 min, purity: 99.38%

¹H NMR (400 MHz, DMSO-d₆) δ=8.06 (d, J=1.2 Hz, 1H), 7.63 (t, J=74.4 Hz, 1H), 7.55-7.47 (m, 3H), 7.34 (dd, J=1.6, 8.4 Hz, 1H), 7.15-7.00 (m, 3H), 5.07 (s, 2H), 3.93 (s, 3H), 3.82 (s, 2H), 2.89 (br d, J=11.2 Hz, 2H), 2.81-2.70 (m, 1H), 2.08 (br t, J=10.8 Hz, 2H), 1.61-1.47 (m, 2H), 1.41 (br d, J=11.2 Hz, 2H)

### Example A185—Preparation of Compound 141. 4-(Difluoromethoxy)-2-((4-(6-((2-fluoro-4-(oxetan-3-yl)benzyl)oxy)pyridin-2-yl)piperidin-1-yl)methyl)-1-methyl-1H-benzo[d]imidazole-6-carboxylic acid (Compound 141) (8)

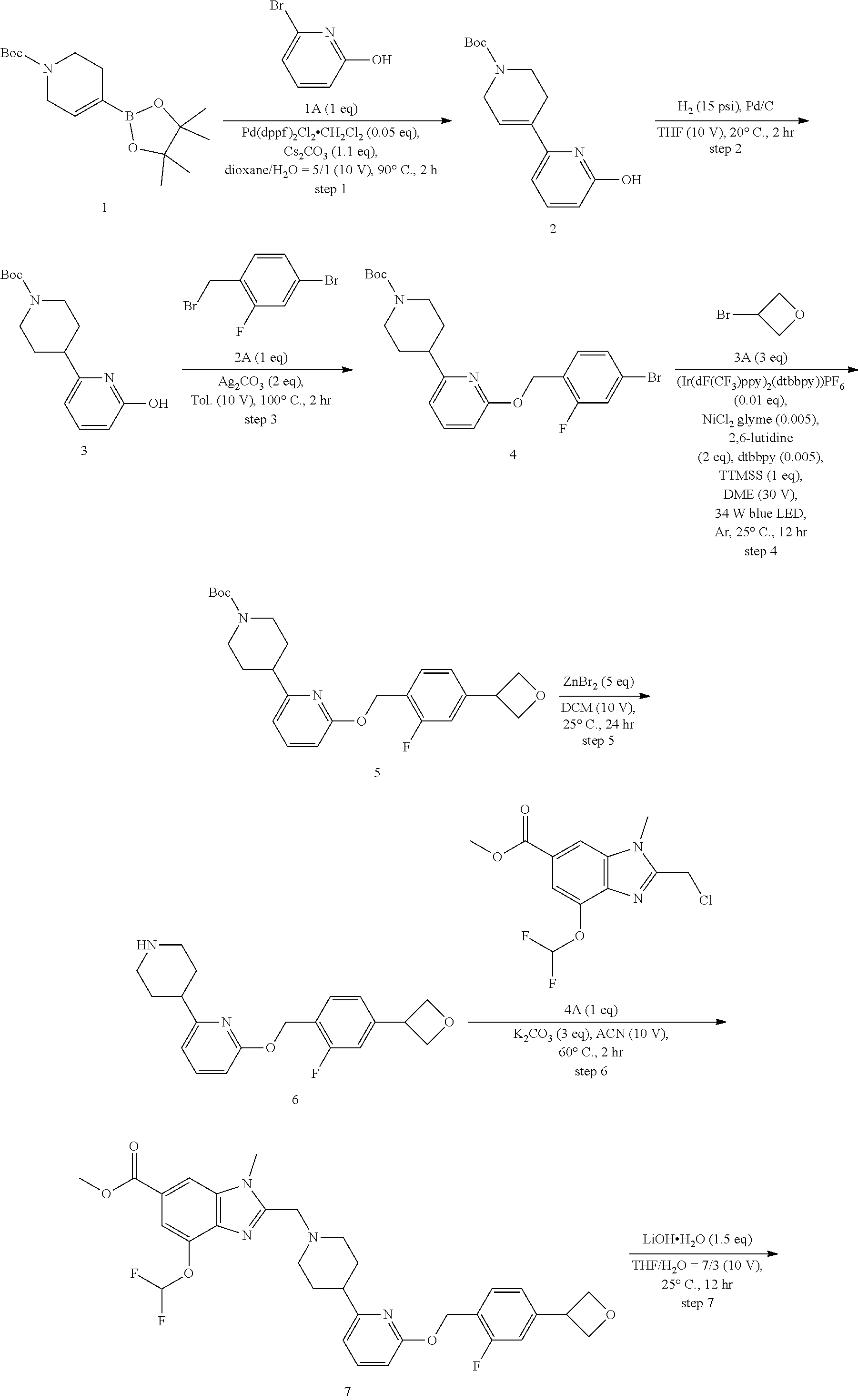

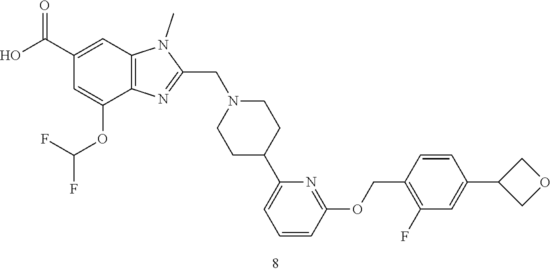 [SMILES: Cn1c(CN2CCC(c3cccc(OCc4ccc(C5COC5)cc4F)n3)CC2)nc2c(OC(F)F)cc(C(=O)O)cc21]

### General Procedure for Preparation of Intermediate 2:

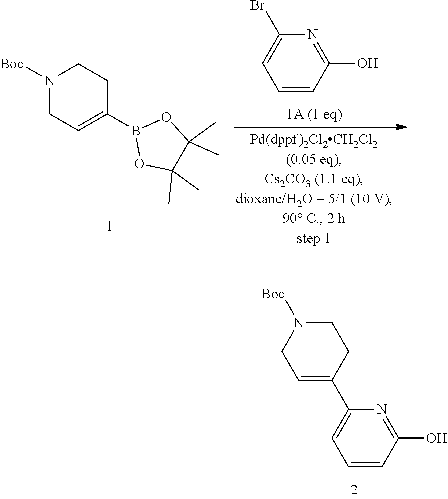 [SMILES: CC(C)(C)OC(=O)N1CC=C(B2OC(C)(C)C(C)(C)O2)CC1 ; Oc1cccc(Br)n1 ; CC(C)(C)OC(=O)N1CC=C(c2cccc(O)n2)CC1]

To a solution of 1 (50 g, 161.70 mmol, 1 eq) and 1A (28.14 g, 161.70 mmol, 1 eq) in dioxane (500 mL) was added Cs₂CO₃ (57.95 g, 177.87 mmol, 1.1 eq) in H₂O (100 mL) and added Pd(dppf)Cl₂·CH₂Cl₂ (6.60 g, 8.09 mmol, 0.05 eq) at 20° C. The mixture was stirred at 90° C. for 2 hr. LCMS (product: RT=1.363 min) showed the 1 was consumed completely. The reaction mixture was cooled down to 20° C. and quenched by addition H₂O 500 mL at 20° C., and then extracted with EtOAc 300 mL*3. The combined organic layers were washed with brine 200 mL*2, dried over Na₂SO₄, filtered, and concentrated under reduced pressure to give a residue. The residue was purified by column chromatography (SiO₂, Petroleum ether/Ethyl acetate=100/1 to 0/1). 2 (34 g, 123.04 mmol, 76.09% yield) was obtained as a white solid.

¹H NMR (400 MHz, CHCl3-d) δ=7.49-7.38 (m, 1H), 6.52-6.43 (m, 2H), 6.21 (d, J=5.6 Hz, 1H), 4.15 (s, 2H), 3.63 (s, 2H), 2.54-2.37 (m, 2H), 1.50 (s, 9H)

### General Procedure for Preparation of Intermediate 3:

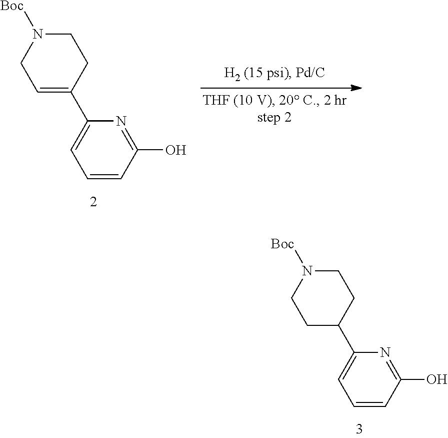 [SMILES: CC(C)(C)OC(=O)N1CC=C(c2cccc(O)n2)CC1 ; CC(C)(C)OC(=O)N1CCC(c2cccc(O)n2)CC1]

THF (300 mL) was charged to the hydrogenating flask, then Pd/C (5 g) was added to the solution at 25° C. 2 (27 g, 97.71 mmol, 1 eq) was added to the reaction mixture, under Ar atmosphere. The mixture was degassed under vacuum and purged with H₂ 3 times. The mixture was stirred under H₂ (15 psi) at 25° C. for 2 hr. LCMS (product: RT=1.349 min) showed 2 was consumed completely. The suspension was filtered through a pad of Celite, and the filtered cake was washed with MeOH 100 mL×5. The combined filtrate was concentrated to give a product. Compound 3 (26 g, 93.41 mmol, 95.60% yield) was obtained as a yellow solid.

¹H NMR (400 MHz, CHCl3-d) δ=7.55-7.33 (m, 1H), 6.52-6.35 (m, 1H), 6.06 (d, J=6.0 Hz, 1H), 4.40-4.19 (m, 2H), 2.91-2.78 (m, 2H), 2.76-2.63 (m, 1H), 1.95 (d, J=12.4 Hz, 2H), 1.70-1.54 (m, 2H), 1.48 (s, 9H)

### General Procedure for Preparation of Intermediate 4:

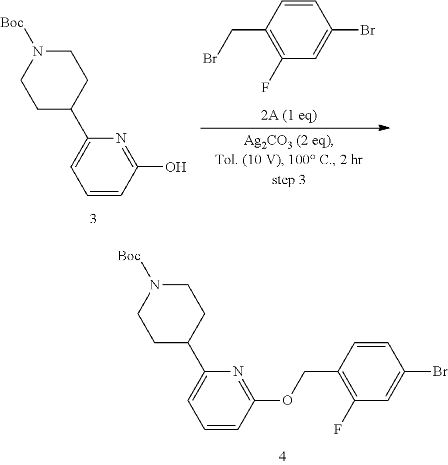 [SMILES: CC(C)(C)OC(=O)N1CCC(c2cccc(O)n2)CC1 ; Fc1cc(Br)ccc1CBr ; CC(C)(C)OC(=O)N1CCC(c2cccc(OCc3ccc(Br)cc3F)n2)CC1]

A mixture of 3 (1 g, 3.59 mmol, 1 eq), 2A (962.55 mg, 3.59 mmol, 1 eq), Ag₂CO₃ (1.98 g, 7.19 mmol, 326.03 μL, 2 eq) in toluene (10 mL) was degassed and purged with N₂ for 3 times, and then the mixture was stirred at 100° C. for 2 hr under N₂ atmosphere. LCMS (product: RT=0.720 min) showed the 3 was consumed completely. The reaction mixture was cooled down to 30° C. and filtered. The filtrate was added H₂O 10 mL and extracted with EtOAc (10 mL*3). The combined organic layers were washed with brine 20 mL (10 mL*2), dried over Na₂SO₄, filtered and concentrated under reduced pressure to give a residue. The residue was purified by column chromatography (SiO₂, Petroleum ether/Ethyl acetate=10/1 to 1/1). 4 (1.2 g, 2.58 mmol, 71.78% yield) was obtained as a colorless oil.

### General Procedure for Preparation of Intermediate 5:

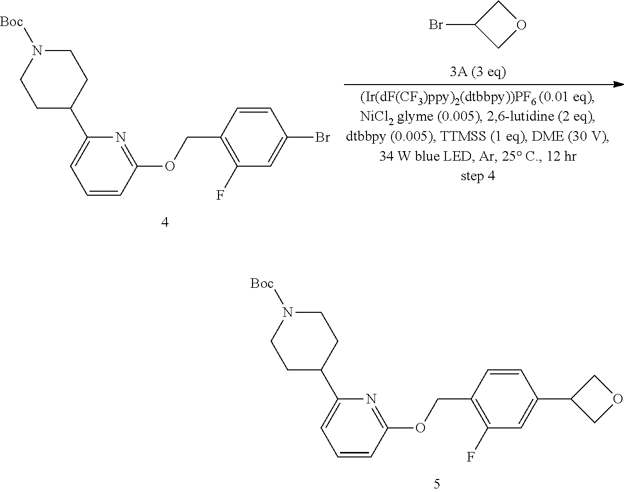 [SMILES: CC(C)(C)OC(=O)N1CCC(c2cccc(OCc3ccc(Br)cc3F)n2)CC1 ; BrC1COC1 ; CC(C)(C)OC(=O)N1CCC(c2cccc(OCc3ccc(C4COC4)cc3F)n2)CC1]

A mixture of 4 (600 mg, 1.29 mmol, 1 eq), 3A (529.82 mg, 3.87 mmol, 3 eq), (Ir(dF(CF₃)ppy)₂(dtbbpy))PF₆ (14.47 mg, 12.89 μmol, 0.01 eq), NiCl₂ glyme (1.42 mg, 6.45 μmol, 0.005 eq), TTMSS (320.61 mg, 1.29 mmol, 397.78 μL, 1 eq), 2,6-lutidine (276.31 mg, 2.58 mmol, 300.33 μL, 2 eq) and dtbbpy (1.73 mg, 6.45 μmol, 0.005 eq) in DME (18 mL) was degassed and purged with N₂ for 3 times, and then the mixture was stirred at 25° C. for 12 hr at 34W blue LED under Ar atmosphere. LCMS (product: RT=1.534 min) showed the 4 was consumed completely. The reaction mixture was quenched by addition H₂O 8 mL at 20° C. and extracted with EtOAc 24 mL (8 mL*3). The combined organic layers were washed with brine 20 mL (10 mL*2), dried over Na₂SO₄, filtered and concentrated under reduced pressure to give a residue. The residue was purified by column chromatography (SiO₂, Petroleum ether/Ethyl acetate=5/1 to 1/1). 5 (400 mg, 903.91 μmol, 70.11% yield) was obtained as a white solid.

¹H NMR (400 MHz, CHCl3-d) δ=7.51 (q, J=7.2 Hz, 2H), 7.19-7.13 (m, 2H), 6.73 (d, J=7.2 Hz, 1H), 6.63 (d, J=8.4 Hz, 1H), 5.44 (s, 2H), 5.08 (dd, J=6.0, 8.4 Hz, 2H), 4.75 (t, J=6.4 Hz, 2H), 4.30-4.14 (m, 2H), 2.84 (t, J=11.6 Hz, 2H), 2.75 (tt, J=3.6, 11.6 Hz, 1H), 1.93-1.83 (m, 2H), 1.73 (dq, J=4.4, 12.4 Hz, 2H), 1.50 (s, 9H)

### General Procedure for Preparation of Intermediate 6:

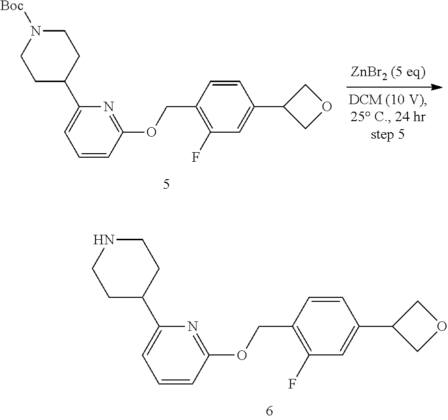 [SMILES: CC(C)(C)OC(=O)N1CCC(c2cccc(OCc3ccc(C4COC4)cc3F)n2)CC1 ; Fc1cc(C2COC2)ccc1COc1cccc(C2CCNCC2)n1]

A mixture of 5 (400 mg, 903.91 μmol, 1 eq), ZnBr₂ (407.12 mg, 1.81 mmol, 90.47 μL, 2 eq) in DCM (4 mL) was degassed and purged with N₂ for 3 times, and then the mixture was stirred at 25° C. for 12 hr under N₂ atmosphere. The reaction mixture was added ZnBr₂ (203.56 mg, 903.91 μmol, 45.24 μL, 1 eq), and then the mixture was stirred at 25° C. for 4 hr under N₂ atmosphere. The reaction mixture was added ZnBr₂ (203.56 mg, 903.91 μmol, 45.24 μL, 1 eq), and then the mixture was stirred at 25° C. for 4 hr under N₂ atmosphere. The reaction mixture was added ZnBr₂ (203.56 mg, 903.91 μmol, 45.24 μL, 1 eq), and was stirred at 25° C. for 4 hr under N₂ atmosphere. LCMS (product: RT=0.359 min) showed 5 was consumed completely. The mixture was filtered, and filtrate was concentrated under reduced pressure to give a residue. The residue was diluted with CH₃CN 2 mL and filtered. The residue was purified by prep-HPLC (column: Waters Xbridge Prep OBD C18 150*40 mm*10 um; mobile phase: [H₂O (10 mM NH₄HCO₃)—CH₃CN]; gradient: 15%-45% B over 8.0 min). 6 (60 mg, 175.23 μmol, 19.39% yield) was obtained as a white solid.

¹H NMR (400 MHz, CHCl3-d) δ=7.54-7.48 (m, 2H), 7.18-7.12 (m, 2H), 6.74 (d, J=7.2 Hz, 1H), 6.62 (d, J=8.0 Hz, 1H), 5.45 (s, 2H), 5.08 (dd, J=6.0, 8.3 Hz, 2H), 4.74 (t, J=6.4 Hz, 2H), 4.28-4.15 (m, 1H), 3.24 (d, J=12.4 Hz, 2H), 2.84-2.68 (m, 3H), 1.96-1.89 (m, 2H), 1.85-1.74 (m, 3H)

### General Procedure for Preparation of Intermediate 7:

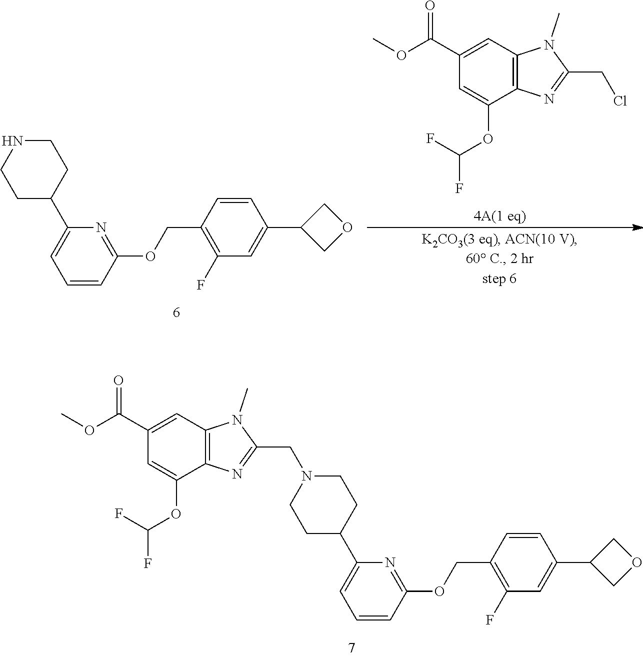 [SMILES: Fc1cc(C2COC2)ccc1COc1cccc(C2CCNCC2)n1 ; COC(=O)c1cc(OC(F)F)c2nc(CCl)n(C)c2c1 ; COC(=O)c1cc(OC(F)F)c2nc(CN3CCC(c4cccc(OCc5ccc(C6COC6)cc5F)n4)CC3)n(C)c2c1]

A mixture of 6 (60 mg, 175.23 μmol, 1 eq), 4A (53.39 mg, 175.23 μmol, 1 eq), K₂CO₃ (72.65 mg, 525.69 μmol, 3 eq) in CH₃CN (0.6 mL) was degassed and purged with N₂ for 3 times, and then the mixture was stirred at 60° C. for 2 hr under N₂ atmosphere. TLC (PE/EtOAc=1/1, product R_{f}=0.33) showed 6 was consumed completely. The reaction mixture was quenched by addition H₂O 3 mL at 20° C., and extracted with EtOAc 9 mL (3 mL*3). The combined organic layers were washed with brine 6 mL (3 mL*2), dried over Na₂SO₄, filtered and concentrated under reduced pressure to give a residue. The residue was purified by column chromatography (SiO₂, Petroleum ether/Ethyl acetate=10/1 to 1/1). 7 (85 mg, 139.20 μmol, 79.44% yield) was obtained as a yellow oil.

LCMS: RT=0.441 min, MS cal.: 610.2, [M+H]⁺=611.2

### General Procedure for Preparation of Compound 141 (8):

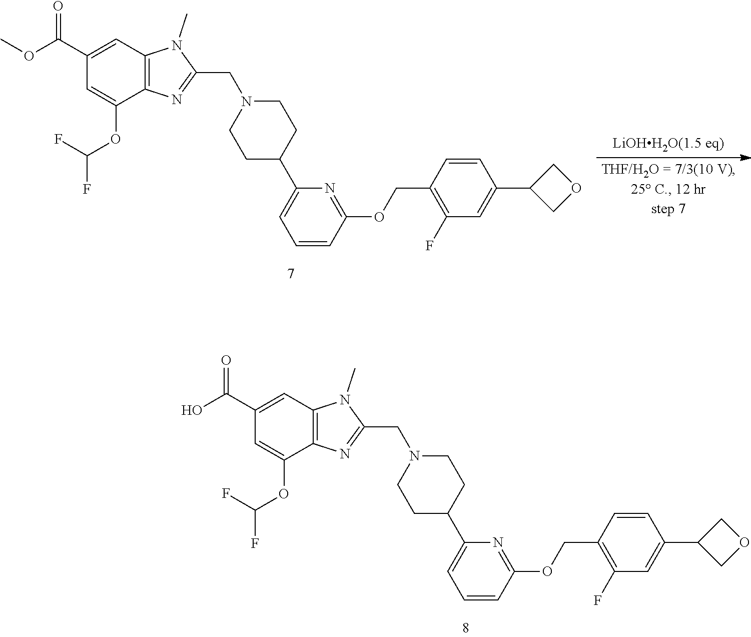 [SMILES: COC(=O)c1cc(OC(F)F)c2nc(CN3CCC(c4cccc(OCc5ccc(C6COC6)cc5F)n4)CC3)n(C)c2c1 ; Cn1c(CN2CCC(c3cccc(OCc4ccc(C5COC5)cc4F)n3)CC2)nc2c(OC(F)F)cc(C(=O)O)cc21]

To a solution of 7 (85 mg, 139.20 μmol, 1 eq), in THF (0.595 mL) was added LiOH. H₂O (8.76 mg, 208.80 μmol, 1.5 eq) in H₂O (0.255 mL). The mixture was stirred at 25° C. for 12 hr under N₂ atmosphere. LCMS (product: RT=0.412 min) showed the 7 was consumed completely. The reaction was purified by prep-HPLC (column: Waters Xbridge BEH C18 100*30 mm*10 um; mobile phase: [H₂O (10 mM NH₄HCO₃)—CH₃CN]; gradient: 20%-55% B over 8.0 min). 4-(Difluoromethoxy)-2-((4-(6-((2-fluoro-4-(oxetan-3-yl)benzyl)oxy)pyridin-2-yl)piperidin-1-yl)methyl)-1-methyl-1H-benzo[d]imidazole-6-carboxylic acid (28.51 mg, 47.55 μmol, 34.16% yield, 99.51% purity) was obtained as a white solid.

LCMS: RT=2.590 min, MS cal.: 596.2, [M+H]⁺=597.3

HPLC: RT=6.450 min, purity: 99.51%

¹H NMR (400 MHz, MeOH-d4) δ=8.10 (s, 1H), 7.68 (s, 1H), 7.56 (t, J=8.0 Hz, 1H), 7.49 (t, J=8.0 Hz, 1H), 7.25 (t, J=74.4 Hz, 1H), 7.18 (s, 1H), 7.16 (d, J=4.8 Hz, 1H), 6.81 (d, J=7.2 Hz, 1H), 6.62 (d, J=8.4 Hz, 1H), 5.41 (s, 2H), 5.04 (dd, J=6.0, 8.4 Hz, 2H), 4.68 (t, J=6.4 Hz, 2H), 4.23 (quin, J=7.6 Hz, 1H), 4.02 (s, 3H), 4.00 (s, 2H), 3.11 (d, J=11.6 Hz, 2H), 2.74-2.62 (m, 1H), 2.50-2.37 (m, 2H), 1.98-1.86 (m, 4H)

### Example A186—Preparation of Compound 143. (S)-2-((4-(6-((4-Cyano-2-fluorobenzyl)oxy)pyridin-2-yl)piperidin-1-yl)methyl)-4-hydroxy-1-(oxetan-2-ylmethyl)-1H-imidazo[4,5-c]pyridine-6-carboxylic acid (Compound 143) (6)

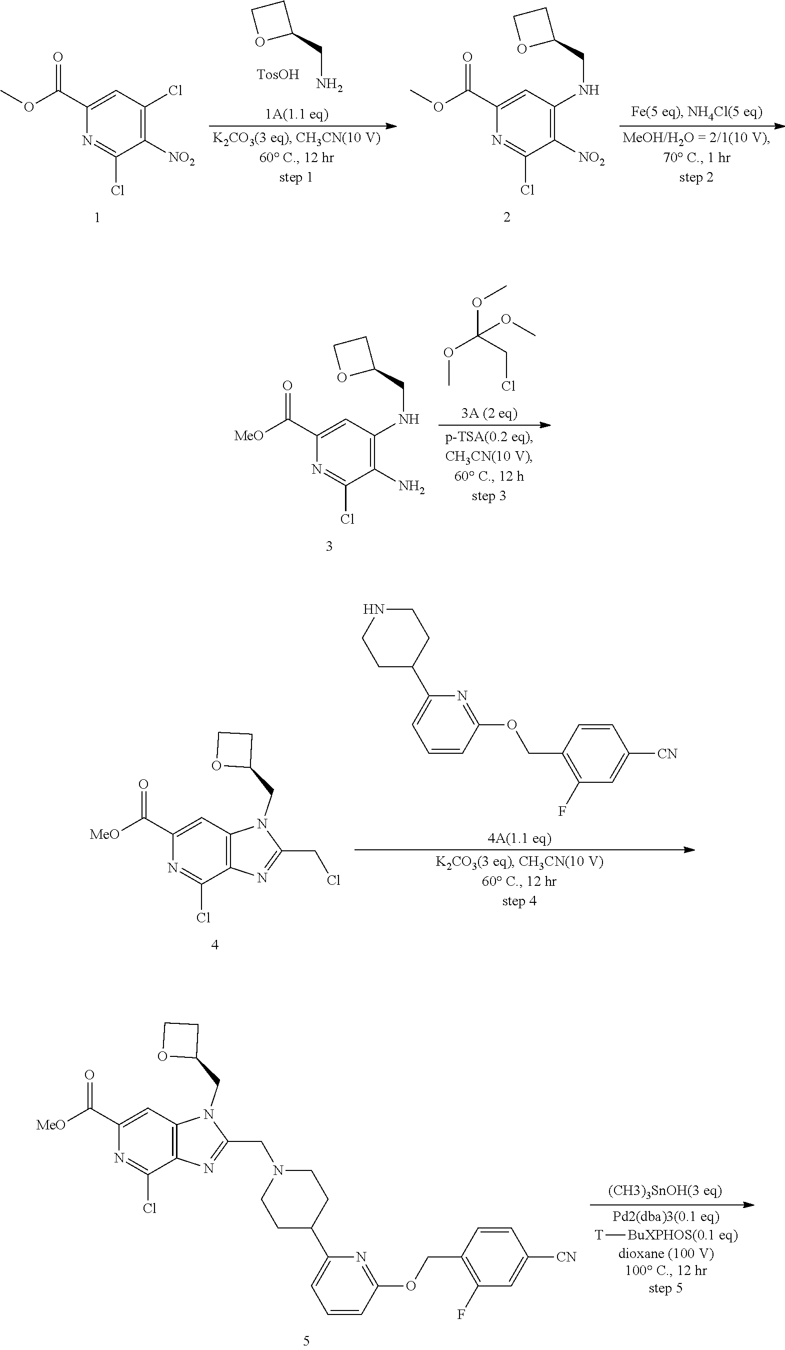

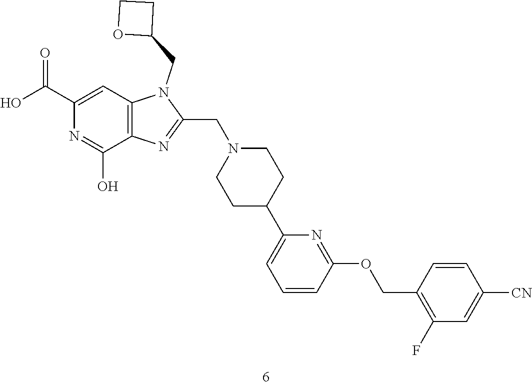 [SMILES: N#Cc1ccc(COc2cccc(C3CCN(Cc4nc5c(O)nc(C(=O)O)cc5n4C[C@@H]4CCO4)CC3)n2)c(F)c1]

### General Procedure for Preparation of Intermediate 2:

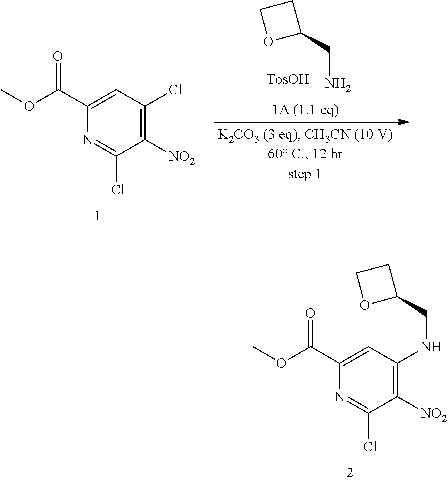

To a solution of 1 (3 g, 11.95 mmol, 1 eq) and 1A (3.41 g, 13.15 mmol, 1.1 eq, p-TSA) in CH₃CN (30 mL) was added K₂CO₃ (4.96 g, 35.85 mmol, 3 eq). The mixture was stirred at 60° C. for 12 hr. LC-MS showed 1 was consumed completely and one main peak with desired m/z was detected. TLC (Petroleum ether:Ethyl acetate=5:1) indicated 1 was consumed completely and one new spot formed. The reaction was clean according to TLC. The reaction mixture was partitioned between Ethyl acetate 60 mL and H₂O 30 mL. The organic phase was separated, washed with brine 20 mL, dried over Na₂SO₄, filtered, and concentrated under reduced pressure to give a residue. The residue was purified by column chromatography (SiO₂, Petroleum ether/Ethyl acetate=100/0 to 60/40). 2 (1.79 g, 5.93 mmol, 49.65% yield) was obtained as a yellow solid.

LCMS: RT=0.955 min, MS cal.: 301.05, 303.04, [M+H]⁺=301.9

¹H NMR (400 MHz, CHCl3-d) δ=6.64 (br s, 1H), 4.81-4.74 (m, 1H), 4.40 (dt, J=6.4, 8.0 Hz, 1H), 4.24 (td, J=6.0, 9.2 Hz, 1H), 3.66 (s, 3H), 3.30-3.25 (m, 2H), 2.44 (dtd, J=6.0, 8.0, 11.6 Hz, 1H), 2.21 (tdd, J=7.2, 9.2, 11.6 Hz, 1H)

### General Procedure for Preparation of Intermediate 3:

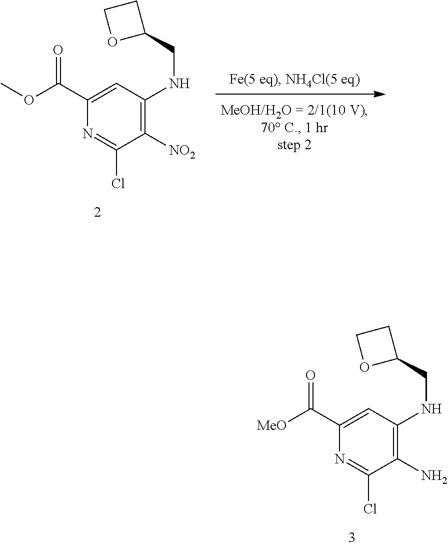 [SMILES: COC(=O)c1cc(NC[C@@H]2CCO2)c([N+](=O)[O-])c(Cl)n1 ; COC(=O)c1cc(NC[C@@H]2CCO2)c(N)c(Cl)n1]

MeOH (12 mL) and H₂O (6 mL) was charged to the 50 mL one-necked round bottom flask, then 2 (1.17 g, 3.88 mmol, 1 eq) and NH₄Cl (1.04 g, 19.39 mmol, 5 eq) was added to the mixture. At 25° C., Fe (1.08 g, 19.39 mmol, 5 eq) was added dropwise to the reaction mixture. After the addition, the mixture was stirred at 70° C. for 1 hr. LCMS showed 2 was consumed completely and one main peak with desired m/z was detected. The reaction mixture was filtered and the filter cake was washed with MeoH (50 ml). The filtrate was diluted with H₂O 20 mL and extracted with Ethyl acetate 40 mL (20 mL*2). The combined organic layers were washed with brine 10 mL, dried over Na₂SO₄, filtered, and concentrated under reduced pressure to give a residue. 3 (1.3 g, crude) was obtained as a green oil.

LCMS: RT=0.747 min, MS cal.: 271.07, 273.07, [M+H]⁺=271.9

¹H NMR (400 MHz, CHCl3-d) δ=7.39 (s, 1H), 5.17-5.04 (m, 1H), 4.75 (q, J=7.2 Hz, 1H), 4.69-4.52 (m, 2H), 3.94 (s, 3H), 3.64-3.39 (m, 3H), 2.85-2.70 (m, 1H), 2.59-2.49 (m, 1H)

### General Procedure for Preparation of Intermediate 4:

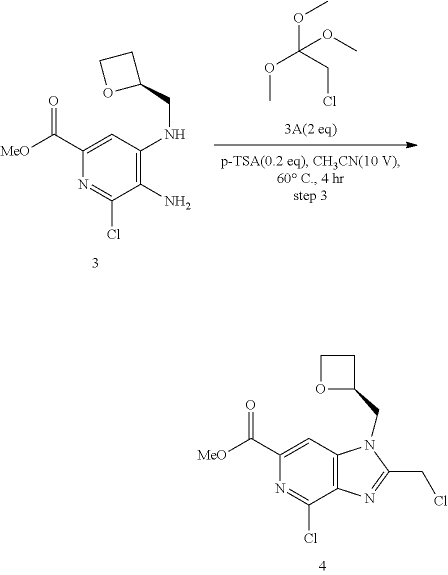

To a solution of 3 (370 mg, 1.36 mmol, 1 eq) in ACN (3.7 mL) was added p-TSA (46.90 mg, 272.36 μmol, 0.2 eq) and 3A (421.05 mg, 2.72 mmol, 367.08 μL, 2 eq). The mixture was stirred at 60° C. for 4 hr. LC-MS showed 3 was consumed completely and one main peak with desired m/z was detected. The reaction mixture was concentrated under reduced pressure to remove ACN to give a residue. The residue was purified by column chromatography (SiO₂, Petroleum ether/Ethyl acetate=4/1 to 0/1). 5 (440 mg, crude) was obtained as a yellow solid, the crude product was used to next step without further purification.

LCMS: RT=0.365 min, MS cal.: 329.03, 331.03, [M+H]⁺=330.0

### General Procedure for Preparation of Intermediate 5:

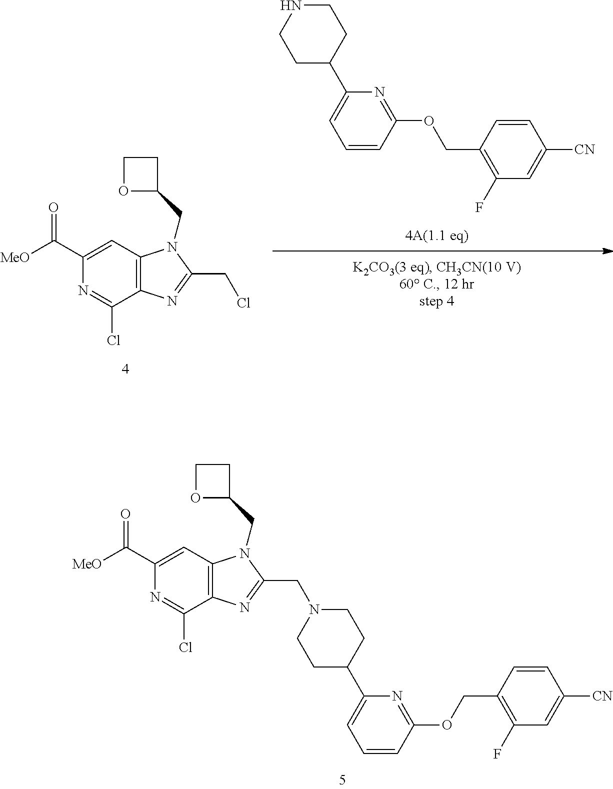 [SMILES: COC(=O)c1cc2c(nc(CCl)n2C[C@@H]2CCO2)c(Cl)n1 ; N#Cc1ccc(COc2cccc(C3CCNCC3)n2)c(F)c1 ; COC(=O)c1cc2c(nc(CN3CCC(c4cccc(OCc5ccc(C#N)cc5F)n4)CC3)n2C[C@@H]2CCO2)c(Cl)n1]

To a solution of 4 (352.69 mg, 1.13 mmol, 1.1 eq) and 4A (340 mg, 1.03 mmol, 1.1 eq) in CH₃CN (1 mL) was added K₂CO₃ (426.97 mg, 3.09 mmol, 3 eq). The mixture was stirred at 60° C. for 12 hr. LCMS showed 4 was consumed completely and one main peak with desired m/z was detected. TLC (Petroleum ether:Ethyl acetate=0:1) indicated 4 was consumed completely and one new spot formed. The reaction was clean according to TLC. After 12 hr, the reaction mixture was diluted with H₂O (10 mL), extracted by Ethyl acetate 20 mL (10 mL*2). Then organic phase was combined and washed by H₂O 10 mL, aq. brine 10 mL, dried with Na₂SO₄, filtered. The organic phase was concentrated under reduced pressure at 40° C. to give a residue. The residue was purified by column chromatography (SiO₂, Petroleum ether/Ethyl acetate=100/0 to 48/52). 5 (550 mg, 818.10 μmol, 79.44% yield, 90% purity) was obtained as a white solid.

LCMS: RT=0.418 min, MS cal.: 604.20, 605.20, [M+H]⁺=605.2

¹H NMR (400 MHz, CHCl3-d) δ=8.54 (s, 1H), 7.90 (d, J=12.0 Hz, 1H), 7.74-7.69 (m, 2H), 7.66 (t, J=8.0 Hz, 1H), 6.90 (d, J=7.2 Hz, 1H), 6.74 (d, J=8.0 Hz, 1H), 5.48 (s, 2H), 5.18-5.10 (m, 1H), 4.99-4.89 (m, 1H), 4.77 (dd, J=2.4, 15.2 Hz, 1H), 4.54-4.45 (m, 1H), 4.39 (td, J=6.0, 9.2 Hz, 1H), 4.08-3.98 (m, 1H), 3.88 (d, J=14.0 Hz, 1H), 3.00 (br d, J=10.8 Hz, 1H), 2.85 (br d, J=11.2 Hz, 1H), 2.77-2.67 (m, 1H), 2.60 (ddd, J=3.6, 8.0, 15.2 Hz, 2H), 2.49-2.38 (m, 2H), 2.34-2.14 (m, 2H), 1.83-1.72 (m, 3H), 1.72-1.59 (m, 2H)

### General Procedure for Preparation of Compound 143 (6):

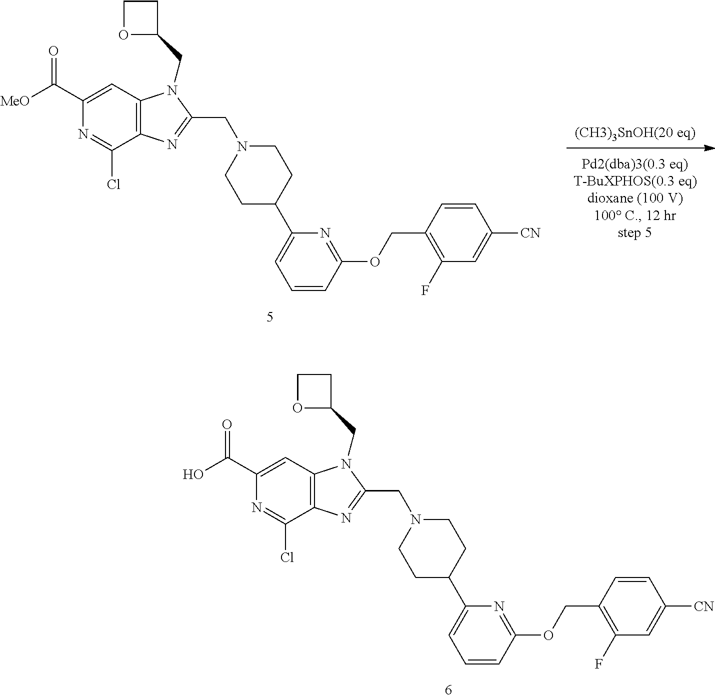 [SMILES: COC(=O)c1cc2c(nc(CN3CCC(c4cccc(OCc5ccc(C#N)cc5F)n4)CC3)n2C[C@@H]2CCO2)c(Cl)n1 ; N#Cc1ccc(COc2cccc(C3CCN(Cc4nc5c(Cl)nc(C(=O)O)cc5n4C[C@@H]4CCO4)CC3)n2)c(F)c1]

2 reactions were carried out in parallel.

To a solution of 5 (100 mg, 165.27 μmol, 1 eq) in dioxane (1 mL) was added Pd₂(dba)₃ (45.40 mg, 49.58 μmol, 0.3 eq) and hydroxy (trimethyl) stannane (597.70 mg, 3.31 mmol, 20 eq) and t-Bu Xphos (21.05 mg, 49.58 μmol, 0.3 eq). The mixture was stirred at 100° C. for 12 hr. LCMS showed 5 remained 30% and three main peak with desired m/z was detected. 2 reactions were combined to be worked up. The reaction mixture was added KF 3 ml at 25° C. within 10 min, filtered and the filter cake was washed with Ethyl acetate 5 mL. The combined filtrates were concentrated to dryness to give product as a orange solid. The residue was purified by prep-HPLC (column: Waters Xbridge Prep OBD C18 150*40 mm*10 um; mobile phase: [H₂O (10 mM NH₄HCO₃)-ACN]; gradient: 20%-50% B over 8.0 min). (S)-2-((4-(6-((4-Cyano-2-fluorobenzyl)oxy)pyridin-2-yl)piperidin-1-yl)methyl)-4-hydroxy-1-(oxetan-2-ylmethyl)-1H-imidazo[4,5-c]pyridine-6-carboxylic acid (7.06 mg, 6.62 μmol, 2.00% yield, 97% purity) was obtained as a white solid.

LCMS: RT=2.527 min, MS cal.: 572.22, 573.22, [M+H]⁺=573.3

HPLC: RT=8.510 min, purity: 97.28%

¹H NMR (400 MHz, MeOH-d4) δ=9.72-9.62 (m, 1H), 7.89 (d, J=12.0 Hz, 1H), 7.71 (d, J=4.0 Hz, 2H), 7.65 (t, J=8.0 Hz, 1H), 7.03 (s, 1H), 6.89 (d, J=7.6 Hz, 1H), 6.72 (d, J=8.0 Hz, 1H), 5.48 (s, 2H), 5.07 (dq, J=3.2, 7.2 Hz, 1H), 4.68-4.60 (m, 1H), 4.55 (d, J=2.8 Hz, 1H), 4.49-4.43 (m, 1H), 4.37 (td, J=6.0, 9.2 Hz, 1H), 3.83-3.77 (m, 1H), 3.71-3.66 (m, 1H), 2.94 (br d, J=10.8 Hz, 1H), 2.83 (br d, J=11.6 Hz, 1H), 2.70-2.66 (m, 1H), 2.61-2.56 (m, 1H), 2.45-2.39 (m, 1H), 2.21-2.09 (m, 2H), 1.79-1.72 (m, 2H), 1.70-1.61 (m, 2H)

### Example A187—Preparation of Compound 144. 2-((4-(3-((4-Cyano-2-fluorobenzyl)oxy)-2-methoxyphenyl)piperidin-1-yl)methyl)-4-(difluoromethoxy)-1-methyl-1H-benzo[d]imidazole-6-carboxylic acid (Compound 144) (7)

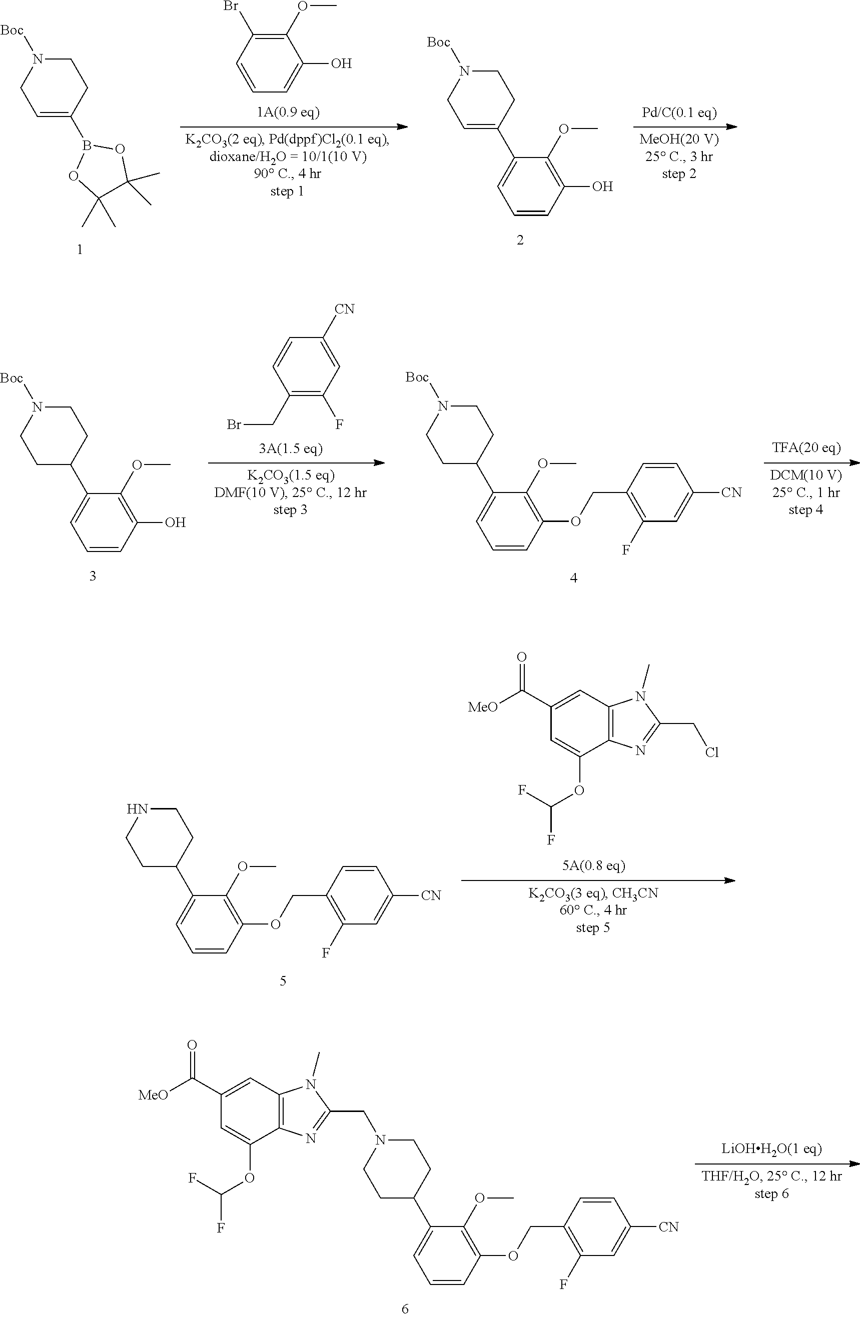

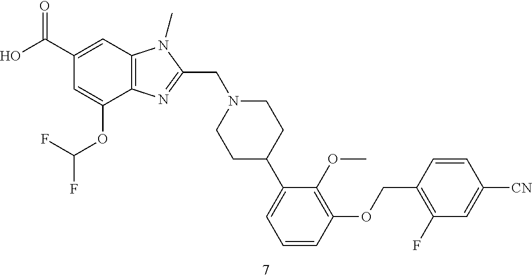 [SMILES: COc1c(OCc2ccc(C#N)cc2F)cccc1C1CCN(Cc2nc3c(OC(F)F)cc(C(=O)O)cc3n2C)CC1]

### General Procedure for Preparation of Intermediate 2:

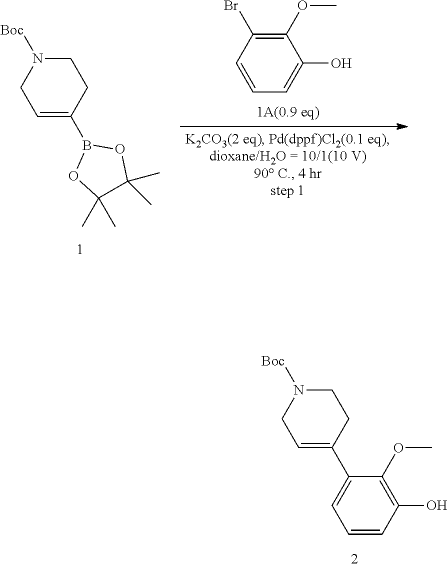

To a mixture of 1 (1.83 g, 5.91 mmol, 1 eq) and 1A (1 g, 4.93 mmol, 0.9 eq) in dioxane (8 mL) and H₂O (2 mL) was added K₂CO₃ (1.36 g, 9.85 mmol, 2 eq) and Pd(dppf)Cl₂ (360.39 mg, 492.53 μmol, 0.1 eq) in one portion at 25° C. The mixture was de-gassed under reduced pressure and recharged with N₂. The mixture was stirred at 90° C. for 4 hours. LC-MS showed 1 was consumed completely and one main peak with desired mass was detected. The aqueous phase was extracted with ethyl acetate (20 mL*3). The combined organic phase was washed with saturated brine (50 mL*1), dried with anhydrous Na₂SO₄, filtered, and concentrated in vacuum. The residue was purified by silica gel chromatography (column height: 250 mm, diameter: 100 mm, 100-200 mesh silica gel, Petroleum ether/Ethyl acetate=100/0, 7/93). 2 (1.3 g, 4.26 mmol, 86.43% yield) as a white oil.

LCMS: RT=0.512 min, MS cal.: 305.2, [M+H−Boc]⁺=206.2

¹H NMR (400 MHz, MeOH-d4) δ=6.90-6.85 (m, 1H), 6.76 (dd, J=1.2, 8.0 Hz, 1H), 6.63 (dd, J=1.2, 7.6 Hz, 1H), 5.81 (br s, 1H), 4.03 (br s, 2H), 3.69 (s, 3H), 3.61 (br s, 2H), 2.49 (br d, J=1.6 Hz, 2H), 1.50 (s, 9H)

### General Procedure for Preparation of Intermediate 3:

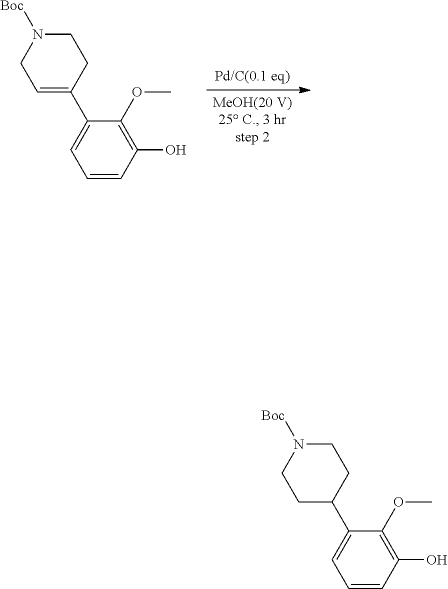 [SMILES: COc1c(O)cccc1C1=CCN(C(=O)OC(C)(C)C)CC1 ; "MeOH(20 V)25° C., 3 hrstep 2" ; "Pd/C(0.1 eq)" ; COc1c(O)cccc1C1CCN(C(=O)OC(C)(C)C)CC1]

To a mixture of 2 (800 mg, 2.62 mmol, 1 eq) in MeOH (16 mL) was added Pd/C (278.80 mg, 261.98 μmol, 10% purity, 0.1 eq) in one portion at 25° C. under N₂. The mixture was de-gassed under reduced pressure and recharged with H₂ (15 Psi). Then, the mixture was stirred at 25° C. for 2 hours under H₂ (15 Psi). LC-MS showed 2 was consumed completely and one main peak with desired mass was detected. The suspension was filtered, and the filter cake was washed with MeOH (10 mL*3). The combined filtrates were concentrated to dryness to give product. 3 (800 mg, 2.60 mmol, 99.34% yield) as a white solid.

LCMS: RT=0.516 min, MS cal.: 307.2, [M+H−t-Bu]⁺=252.1

HPLC: product: RT=3.673 min, purity: 99.61%

¹H NMR (400 MHz, MeOH-d4) δ=6.91-6.85 (m, 1H), 6.69 (dd, J=1.2, 8.0 Hz, 1H), 6.66 (dd, J=1.2, 7.6 Hz, 1H), 4.20 (br d, J=13.2 Hz, 2H), 3.78 (s, 3H), 3.10 (tt, J=3.2, 12.0 Hz, 1H), 2.87 (br s, 2H), 1.74 (br d, J=12.8 Hz, 2H), 1.57 (dq, J=4.0, 12.8 Hz, 2H), 1.48 (s, 9H)

### General Procedure for Preparation of Intermediate 4:

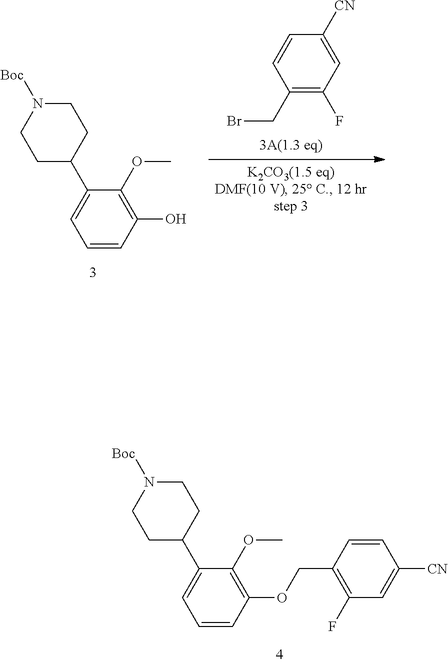

To a mixture of 3 (300 mg, 975.98 μmol, 1 eq) and 3A (271.56 mg, 1.27 mmol, 1.3 eq) in DMF (3 mL) was added K₂CO₃ (202.34 mg, 1.46 mmol, 1.5 eq) in one portion at 25° C. The mixture was stirred at 25° C. for 2 hours. LC-MS showed 3 was consumed completely and one main peak with desired mass was detected. The suspension was filtered, the mixture was concentrated in vacuum. The residue was purified by silica gel chromatography (column height: 250 mm, diameter: 100 mm, 100-200 mesh silica gel, Petroleum ether/Ethyl acetate=100/0, 7/93). 4 (360 mg, 817.24 μmol, 83.74% yield) as a white solid.

LCMS: RT=0.636 min, MS cal.: 440.2, [M+H−t-Bu]⁺=385.2

HPLC: product: RT=4.867 min, purity: 99.82%

¹H NMR (400 MHz, MeOH-d4) δ=7.81-7.76 (m, 1H), 7.62 (s, 1H), 7.60 (d, J=1.6 Hz, 1H), 7.04-7.00 (m, 1H), 6.96-6.92 (m, 1H), 6.85 (dd, J=1.6, 7.6 Hz, 1H), 5.24 (s, 2H), 4.20 (br d, J=13.2 Hz, 2H), 3.83 (s, 3H), 3.15 (tt, J=3.2, 12.0 Hz, 1H), 2.88 (br s, 2H), 1.73 (br d, J=12.4 Hz, 2H), 1.64-1.53 (m, 2H), 1.48 (s, 9H)

### General Procedure for Preparation of Intermediate 5:

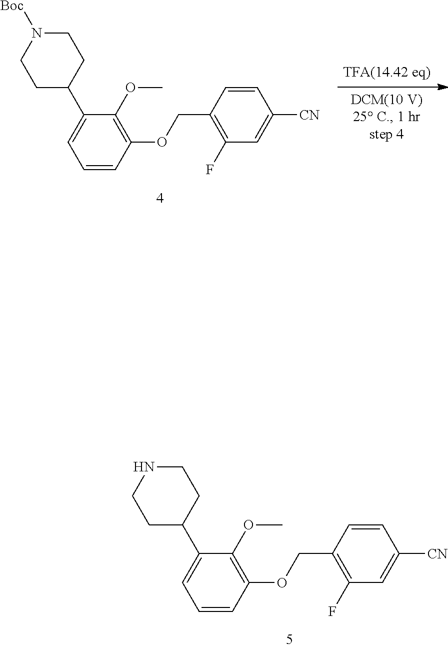 [SMILES: COc1c(OCc2ccc(C#N)cc2F)cccc1C1CCN(C(=O)OC(C)(C)C)CC1 ; "DCM(10 V)25° C., 1 hrstep 4" ; "TFA(14.42 eq)" ; COc1c(OCc2ccc(C#N)cc2F)cccc1C1CCNCC1]

To a mixture of 4 (360 mg, 817.24 μmol, 1 eq) in DCM (4 mL) was added TFA (1.34 g, 11.78 mmol, 875.39 μL, 14.42 eq) in one portion at 25° C. The mixture was stirred at 25° C. for 1 hour. LC-MS showed 4 was consumed completely and one main peak with desired mass was detected. To a mixture was added Saturated sodium carbonate solution to PH=9˜10. The aqueous phase was extracted with DCM (2 mL*3). The combined organic phase was washed with brine (3 mL*2), dried with anhydrous Na₂SO₄, filtered and concentrated in vacuum. 5 (280 mg, 814.44 μmol, 99.66% yield, 99.01% purity) as a white solid.

LCMS: RT=0.375 min, MS cal.: 340.2, [M+H]⁺=341.2

HPLC: product: RT=2.655 min, purity: 99.10%

¹H NMR (400 MHz, CHCl3-d) δ=7.78 (t, J=7.6 Hz, 1H), 7.63 (s, 1H), 7.60 (s, 1H), 7.08-7.04 (m, 1H), 7.01-6.98 (m, 1H), 6.89 (dd, J=1.2, 7.6 Hz, 1H), 5.25 (s, 2H), 3.85 (s, 3H), 3.44 (br d, J=12.4 Hz, 2H), 3.29-3.22 (m, 1H), 3.10 (dt, J=3.2, 12.8 Hz, 2H), 2.00-1.93 (m, 2H), 1.93-1.82 (m, 2H)

### General Procedure for Preparation of Intermediate 6:

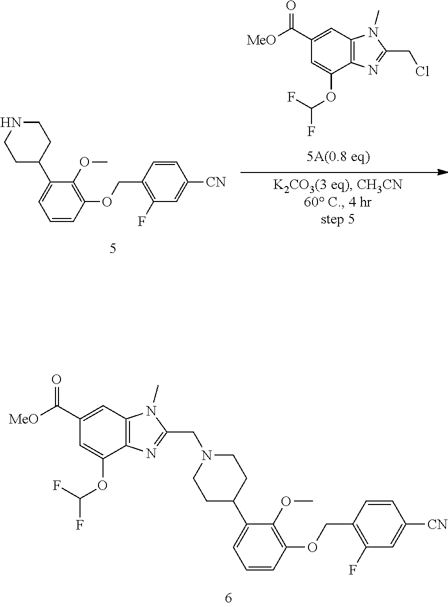 [SMILES: COc1c(OCc2ccc(C#N)cc2F)cccc1C1CCNCC1 ; COC(=O)c1cc(OC(F)F)c2nc(CCl)n(C)c2c1 ; "5A(0.8 eq)" ; "K2CO3(3 eq), CH3CN60° C., 4 hrstep 5" ; COC(=O)c1cc(OC(F)F)c2nc(CN3CCC(c4cccc(OCc5ccc(C#N)cc5F)c4OC)CC3)n(C)c2c1]

To a mixture of methyl Compound 5A (90 mg, 295.40 μmol, 1 eq) and 5 (140.77 mg, 413.55 μmol, 1.4 eq) in ACN (2 mL) was added K₂CO₃ (122.48 mg, 886.19 μmol, 3 eq) in one portion at 25° C. The mixture was stirred at 60° C. for 4 hours. LC-MS showed 4 was consumed completely and one main peak with desired mass was detected. The mixture was filtered and concentrated in vacuum. The crude product was purified by prep-TLC (Petroleum ether:Ethyl acetate=2:1, Rf=0.2). 6 (120 mg, 197.17 μmol, 66.75% yield) as a white solid.

LCMS: RT=0.901 min, MS cal.: 608.2, [M+H]⁺=609.4

HPLC: product: RT=3.507 min, purity: 95.87%

¹H NMR (400 MHz, CHCl3-d) δ=8.13 (d, J=1.2 Hz, 1H), 7.78 (t, J=8.0 Hz, 1H), 7.68 (s, 1H), 7.62 (s, 1H), 7.60 (d, J=2.0 Hz, 1H), 7.26 (t, J=74 Hz, 1H), 7.03-6.98 (m, 1H), 6.94-6.91 (m, 1H), 6.89 (d, J=7.6 Hz, 1H), 5.24 (s, 2H), 4.03 (s, 3H), 3.96 (s, 3H), 3.93 (s, 2H), 3.82 (s, 3H), 3.08-2.95 (m, 3H), 2.41-2.30 (m, 2H), 1.82-1.72 (m, 4H)

### General Procedure for Preparation of Compound 144 (7):

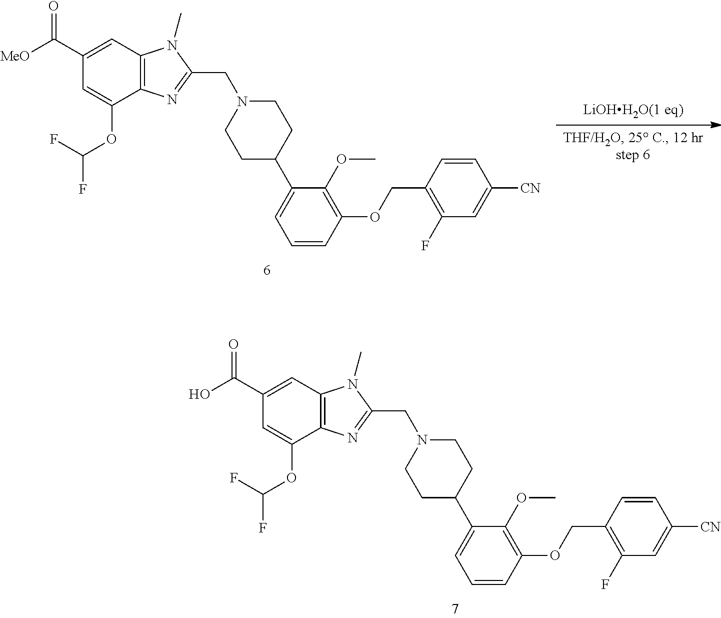 [SMILES: COC(=O)c1cc(OC(F)F)c2nc(CN3CCC(c4cccc(OCc5ccc(C#N)cc5F)c4OC)CC3)n(C)c2c1 ; "LiOH·H2O(1 eq)" ; "THF/H2O, 25° C., 12 hrstep 6" ; COc1c(OCc2ccc(C#N)cc2F)cccc1C1CCN(Cc2nc3c(OC(F)F)cc(C(=O)O)cc3n2C)CC1]

To a mixture of 6 (110 mg, 180.74 μmol, 1 eq) in THF (0.7 mL) and H₂O (0.3 mL) was added LiOH·H₂O (7.58 mg, 180.74 μmol, 1 eq) in one portion at 25° C. The mixture was stirred at 25° C. for 4 hours. LC-MS showed 6 was consumed completely and one main peak with desired mass was detected. The mixture was filtered and concentrated in vacuum. The crude product was purified by prep-HPLC (column: Waters Xbridge Prep OBD C₁₈ 150*40 mm*10 um; mobile phase: [H₂O (10 mM NH₄HCO₃)-ACN]; gradient: 25%-55% B over 8.0 min). 2-((4-(3-((4-Cyano-2-fluorobenzyl)oxy)-2-methoxyphenyl)piperidin-1-yl)methyl)-4-(difluoromethoxy)-1-methyl-1H-benzo[d]imidazole-6-carboxylic acid (28.67 mg, 47.88 μmol, 26.49% yield, 99.305% purity) as a white solid.

LCMS: RT=2.621 min, MS cal.: 594.6, [M+H]⁺=595.2

HPLC: RT=10.813 min, purity: 99.30%

¹H NMR (400 MHz, MeOH-d4) δ=8.09 (d, J=0.8 Hz, 1H), 7.81-7.76 (m, 1H), 7.68 (s, 1H), 7.62 (s, 1H), 7.60 (d, J=2.4 Hz, 1H), 7.23 (t, J=74.4 Hz, 1H), 7.04-6.99 (m, 1H), 6.95-6.88 (m, 2H), 5.24 (s, 2H), 4.02 (s, 3H), 3.97 (s, 2H), 3.82 (s, 3H), 3.09 (br d, J=11.2 Hz, 2H), 3.05-2.97 (m, 1H), 2.40 (dt, J=4.8, 11.2 Hz, 2H), 1.82-1.74 (m, 4H)

### Example A188—Preparation of Compound 145. 2-((4-(2-((4-Cyano-2-fluorobenzyl)oxy)pyrimidin-4-yl)piperidin-1-yl)methyl)-4-(difluoromethoxy)-1-methyl-1H-benzo[d]imidazole-6-carboxylic acid (Compound 145) (6)

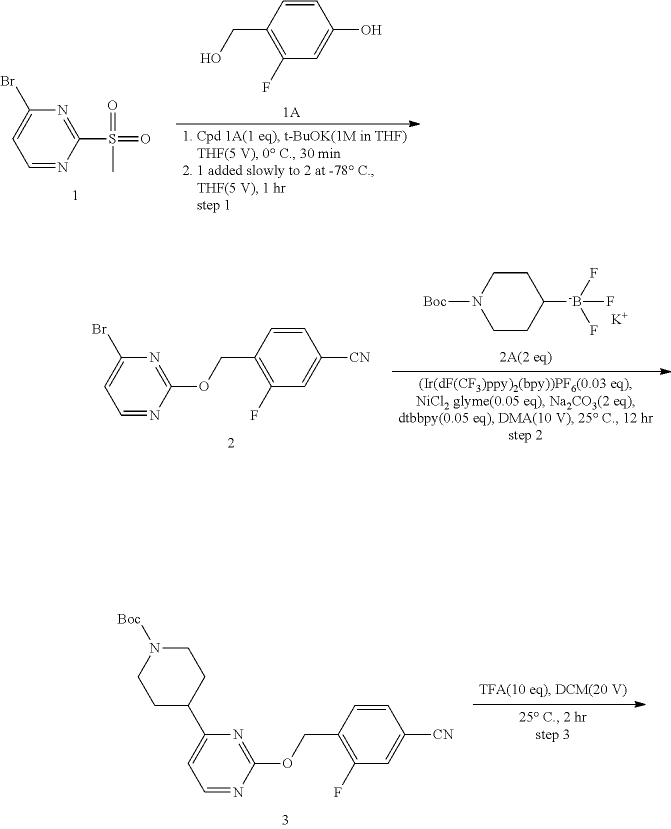

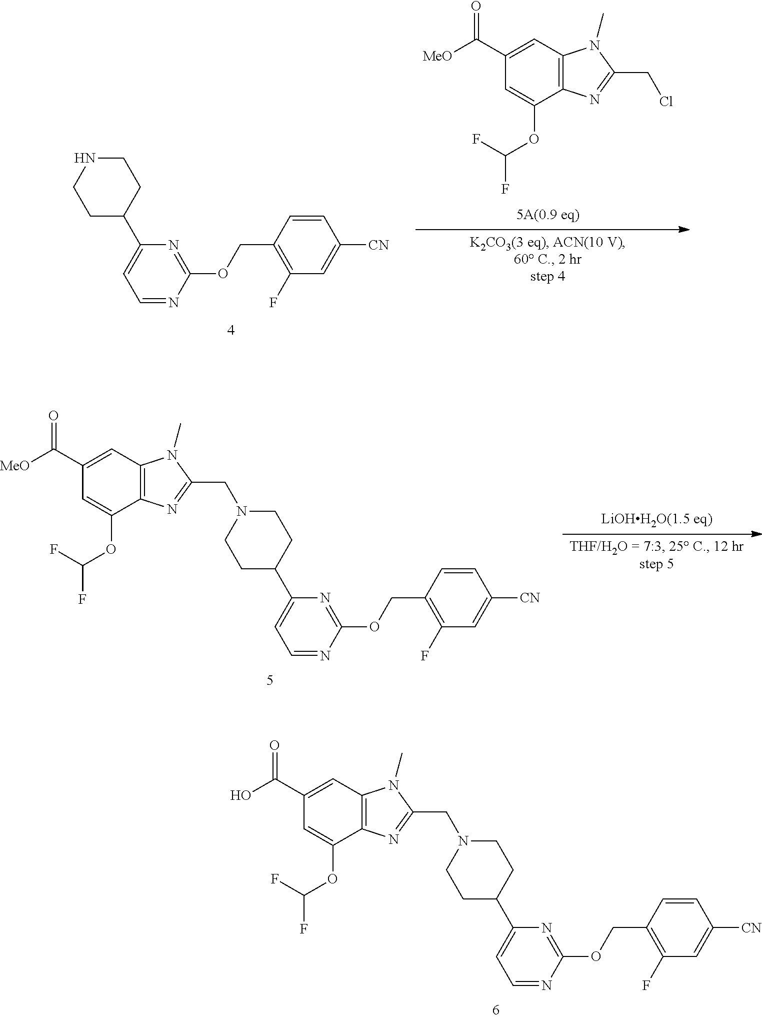

### General Procedure for Preparation of Intermediate 2:

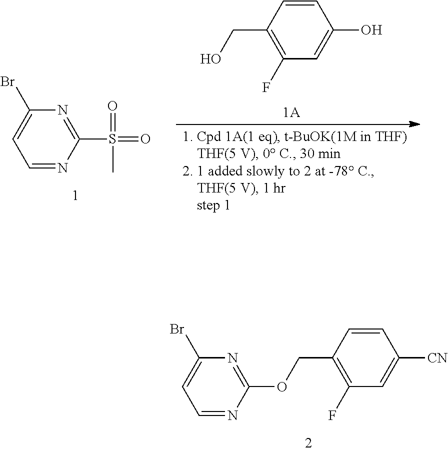

1A (701.26 mg, 4.64 mmol, 1.1 eq) in THF (5 mL) was charged to the round bottom flask at 25° C. Then t-BuOK (1 M, 5.06 mL, 1.2 eq) was added dropwise to the mixture at 0° C. and stirred for 0.5 hr. Then 1 (1 g, 4.22 mmol, 1 eq) in THF (5 mL) and DMA (0.5 mL) was added 1 at −78° C. After the addition, the mixture was stirred for 1.5 hr at −78° C. LC-MS showed 1 was consumed completely and desired mass was detected. The reaction mixture was quenched by addition H₂O 30 mL allowed to warm to 25° C., and then extracted with EtOAc 20 mL. The combined organic layers were washed with brine 20 mL, dried over Na₂SO₄, filtered, and concentrated under reduced pressure to give a residue. The residue was purified by column chromatography (SiO₂, Petroleum ether/Ethyl acetate=1/0 to 0/1). 2 (500 mg, 1.62 mmol, 38.47% yield) was obtained as a white solid.

LCMS: RT=1.856 min, MS cal.: 306.98, [M+H]⁺=308.0

¹H NMR (400 MHz, CHCl3-d) δ=8.35-8.24 (m, 1H), 7.70 (t, J=7.2 Hz, 1H), 7.49 (d, J=8.0 Hz, 1H), 7.40 (d, J=9.2 Hz, 1H), 7.24-7.20 (m, 1H), 5.56 (s, 2H)

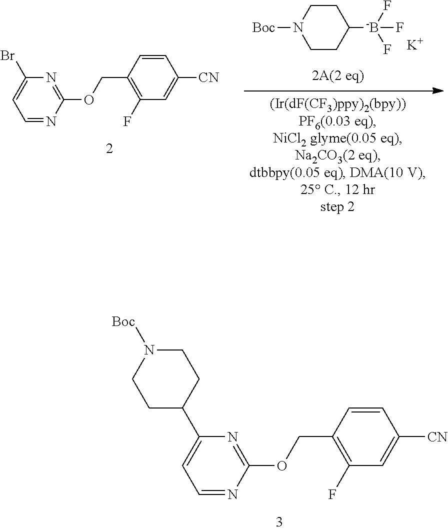

To a solution of 2 (500 mg, 1.62 mmol, 1 eq) in DMA (5 mL) was added NiCl₂·glyme (17.83 mg, 81.14 μmol, 0.05 eq) and Na₂CO₃ (344.00 mg, 3.25 mmol, 2 eq), 2A (945.00 mg, 3.25 mmol, 2 eq, K+), dtbbpy (21.78 mg, 81.14 μmol, 0.05 eq), pentafluoro-phosphane; tetrafluoro-bis(trifluoromethyl)spiro[BLAH]; fluoride (49.16 mg, 48.68 μmol, 0.03 eq) at 25° C. under 34W blue LED. The mixture was stirred at 25° C. for 12 hr. LC-MS showed 2 was consumed completely and desired mass was detected. The reaction mixture was diluted with H₂O 15 mL and extracted with EtOAc 10 mL. The combined organic layers were washed with brine 5 mL, dried over Na₂SO₄, filtered and concentrated under reduced pressure to give a residue. The residue was purified by column chromatography (SiO₂, Petroleum ether/Ethyl acetate=1/0 to 1/1, Rf=0.28). 3 (500 mg, 1.21 mmol, 74.70% yield) was obtained as a white solid.

LCMS: RT=2.123 min, MS cal.: 412.19, [M+H]⁺=413.2

¹H NMR (400 MHz, CHCl3-d) δ=8.44 (d, J=5.2 Hz, 1H), 7.70 (t, J=7.6 Hz, 1H), 7.47 (d, J=7.6 Hz, 1H), 7.39 (d, J=9.2 Hz, 1H), 6.86 (d, J=4.8 Hz, 1H), 5.55 (s, 2H), 4.33-4.16 (m, 2H), 2.89-2.68 (m, 3H), 1.89 (d, J=12.4 Hz, 2H), 1.73 (dd, J=4.4, 12.4 Hz, 2H), 1.48 (s, 9H)

### General Procedure for Preparation of Intermediate 4:

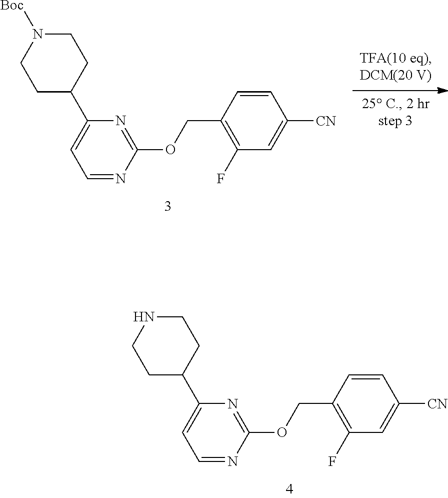 [SMILES: CC(C)(C)OC(=O)N1CCC(c2ccnc(OCc3ccc(C#N)cc3F)n2)CC1 ; "25° C., 2 hrstep 3" ; "TFA(10 eq),DCM(20 V)" ; N#Cc1ccc(COc2nccc(C3CCNCC3)n2)c(F)c1]

To a solution of 3 (400 mg, 969.80 μmol, 1 eq) in DCM (7 mL) was added TFA (1.11 g, 9.70 mmol, 720.39 μL, 10 eq) at 25° C. The mixture was stirred at 25° C. for 2 hr. TLC showed 3 was consumed completely. The reaction mixture was adjusted to pH 8 with NaHCO₃. The aqueous layer was extracted with EtOAc 18 mL. The combined organic phase was washed with brine 12 mL, dried with anhydrous Na₂SO₄, filtered, and concentrated in vacuum. 4 (300 mg, crude) was obtained as a yellow oil.

LCMS: RT=0.837 min, MS cal.: 312.14, [M+H]⁺=313.0

¹H NMR (400 MHz, CHCl3-d) δ=8.51 (d, J=5.0 Hz, 1H), 7.74-7.67 (m, 1H), 7.48 (d, J=7.9 Hz, 1H), 7.40 (dd, J=1.1, 9.2 Hz, 1H), 6.91 (d, J=5.1 Hz, 1H), 5.55 (s, 2H), 3.55 (br d, J=12.7 Hz, 2H), 3.03 (s, 2H), 2.96 (s, 2H), 2.94-2.90 (m, 2H), 2.11 (s, 2H)

### General Procedure for Preparation of Intermediate 5:

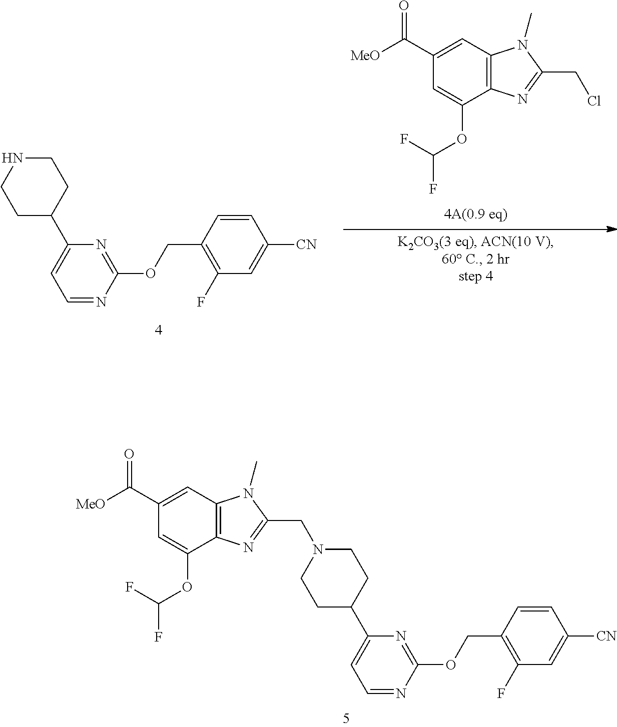 [SMILES: N#Cc1ccc(COc2nccc(C3CCNCC3)n2)c(F)c1 ; COC(=O)c1cc(OC(F)F)c2nc(CCl)n(C)c2c1 ; "4A(0.9 eq)" ; "K2CO3(3 eq), ACN(10 V),60° C., 2 hrstep 4" ; COC(=O)c1cc(OC(F)F)c2nc(CN3CCC(c4ccnc(OCc5ccc(C#N)cc5F)n4)CC3)n(C)c2c1]

To a solution of 4 (100 mg, 320.16 μmol, 1 eq) in ACN (1 mL) was added K₂CO₃ (132.74 mg, 960.49 μmol, 3 eq) and 4A (87.79 mg, 288.15 μmol, 0.9 eq) at 25° C. The mixture was stirred at 60° C. for 2 hr. LCMS showed 4 was consumed completely and desired mass was detected. The reaction mixture was diluted with H₂O 5 mL and extracted with EtOAc 3 mL. The combined organic layers were washed with brine 5 mL, dried over Na₂SO₄, filtered, and concentrated under reduced pressure to give a residue. The residue was purified by column chromatography (SiO₂, Petroleum ether/Ethyl acetate=1/0 to 0/1). 5 (40 mg, 68.90 μmol, 21.52% yield) was obtained as a yellow solid.

LCMS: RT=1.366, MS cal.: 580.57, [M+H]⁺=581.0

¹H NMR (400 MHz, CHCl3-d) δ=8.50 (d, J=5.2 Hz, 1H), 8.11 (s, 1H), 7.91 (d, J=10.0 Hz, 1H), 7.74-7.70 (m, 2H), 7.64 (t, J=74.4 Hz, 1H), 7.53 (s, 1H), 7.10 (d, J=5.2 Hz, 1H), 5.48 (s, 2H), 3.97 (s, 3H), 3.90 (s, 3H), 3.86 (s, 2H), 2.93 (d, J=11.2 Hz, 2H), 2.68-2.61 (m, 1H), 2.54 (s, 1H), 2.22 (t, J=10.4 Hz, 2H), 1.84-1.76 (m, 2H), 1.67 (dd, J=2.8, 12.4 Hz, 2H)

### General Procedure for Preparation of Compound 145 (6):

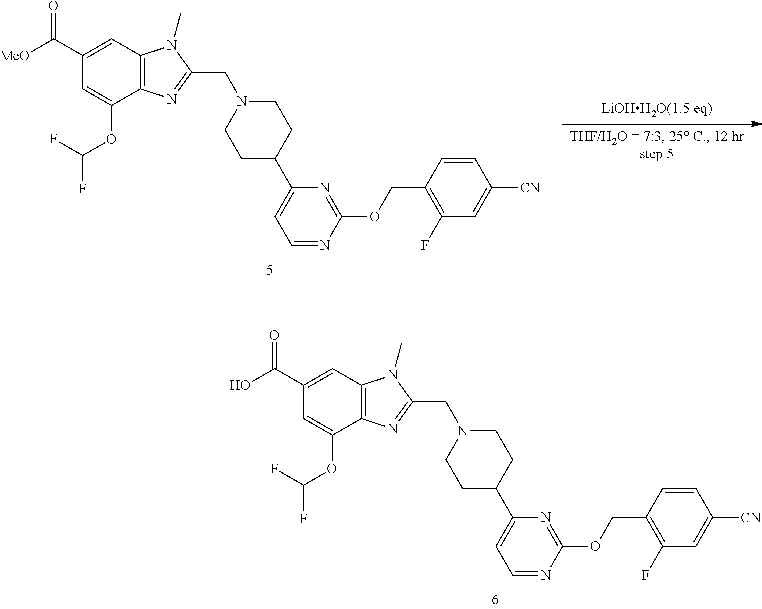

To a solution of 5 (90 mg, 155.02 μmol, 1 eq) in THF (0.7 mL) was added LiOH·H₂O (9.76 mg, 232.54 μmol, 1.5 eq) and H₂O (0.3 mL) at 25° C. The mixture was stirred at 25° C. for 12 hr. LC-MS showed 5 was consumed completely and desired mass was detected. The reaction mixture was filtered to give a residue. The residue was purified by prep-HPLC (column: Waters Xbridge Prep OBD C18 150*40 mm*10 um; mobile phase: [H₂O (10 mM NH₄HCO₃)-ACN]; gradient: 25%-55% B over 8.0 min). 2-((4-(2-((4-Cyano-2-fluorobenzyl)oxy)pyrimidin-4-yl)piperidin-1-yl)methyl)-4-(difluoromethoxy)-1-methyl-1H-benzo[d]imidazole-6-carboxylic acid (26.66 mg, 47.06 μmol, 30.36% yield) was obtained as a white solid.

LCMS: RT=0.966 min, MS cal.: 566.54, [M+H]⁺=567.1.

¹H NMR (400 MHz, CHCl3-d) δ=8.50 (d, J=5.2 Hz, 1H), 8.04 (s, 1H), 7.90 (d, J=10.0 Hz, 1H), 7.71 (d, J=3.2 Hz, 2H), 7.62 (t, J=74.4 Hz, 1H), 7.51 (s, 1H), 7.10 (d, J=5.2 Hz, 1H), 5.48 (s, 2H), 3.94 (s, 3H), 3.85 (s, 2H), 2.93 (d, J=11.6 Hz, 2H), 2.68-2.62 (m, 1H), 2.21 (t, J=11.2 Hz, 2H), 1.84-1.76 (m, 2H), 1.74-1.62 (m, 2H)

### Example A189—Preparation of Compound 146. 2-((4-(2-((4-Cyano-2-fluorobenzyl)oxy)-6-fluorophenyl)piperidin-1-yl)methyl)-4-(difluoromethoxy)-1-methyl-1H-benzo[d]imidazole-6-carboxylic acid (Compound 146) (7)

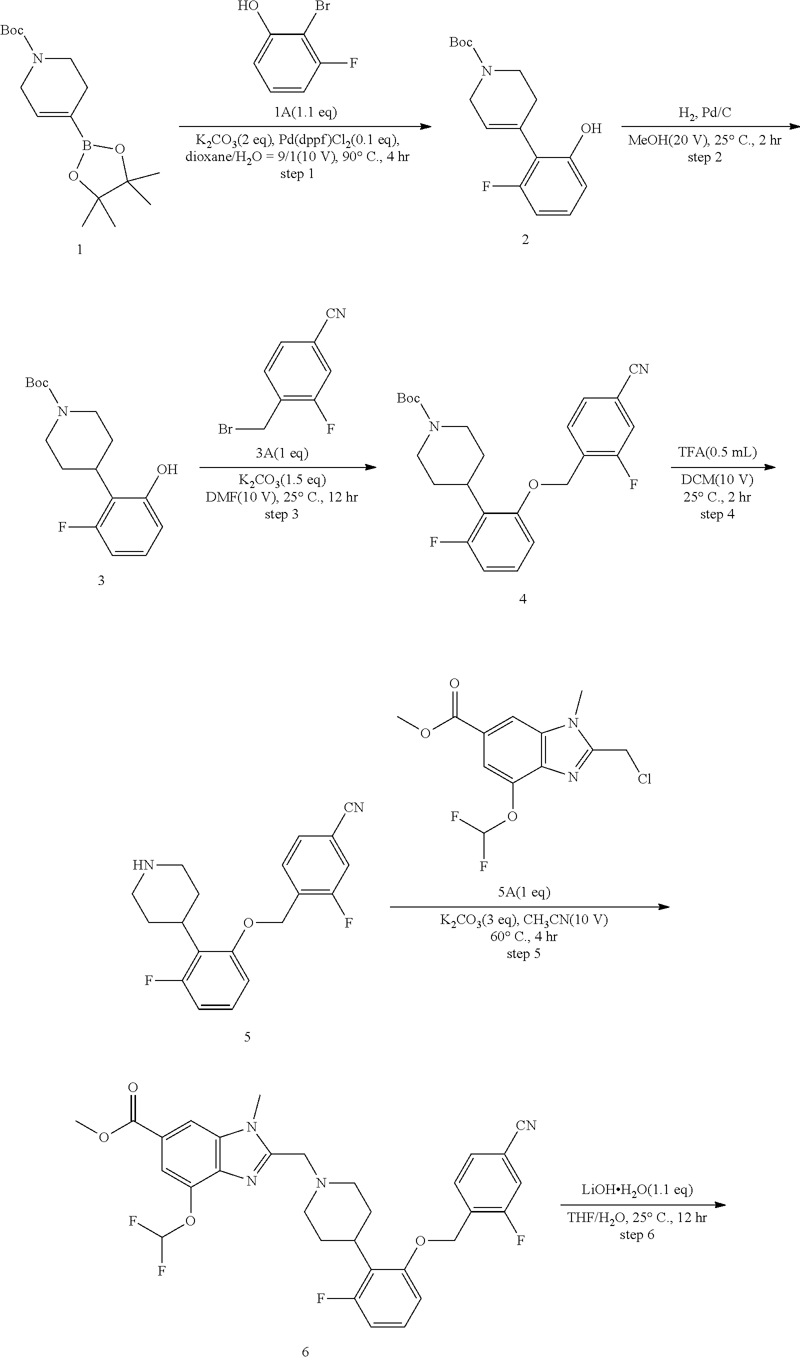

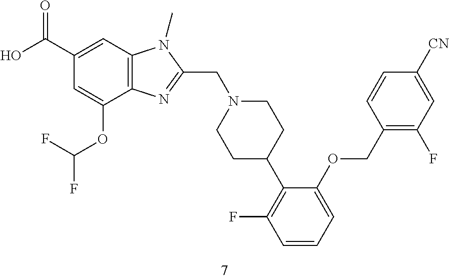 [SMILES: Cn1c(CN2CCC(c3c(F)cccc3OCc3ccc(C#N)cc3F)CC2)nc2c(OC(F)F)cc(C(=O)O)cc21]

### General Procedure for Preparation of Intermediate 2:

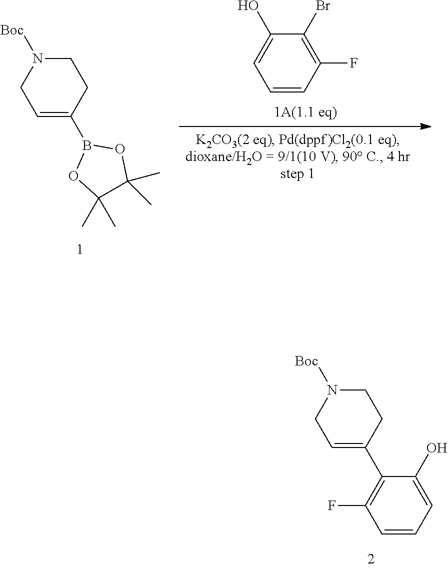

To a mixture of 1 (1 g, 3.23 mmol, 1 eq) and 1A (846.67 mg, 3.56 mmol, 1.1 eq) in dioxane (9 mL) and H₂O (1 mL) was added K₂CO₃ (893.93 mg, 6.47 mmol, 2 eq) and Pd(dppf)Cl₂ (118.32 mg, 161.70 μmol, 0.05 eq) in one portion at 25° C. The mixture was de-gassed under reduced pressure and recharged with N₂. The mixture was stirred at 90° C. for 4 hours. LC-MS showed 1 was consumed completely and one main peak with desired mass was detected. The aqueous phase was extracted with ethyl acetate (20 mL*3). The combined organic phase was washed with saturated brine (50 mL*1), dried with anhydrous Na₂SO₄, filtered and concentrated in vacuum. The residue was purified by silica gel chromatography (column height: 250 mm, diameter: 100 mm, 100-200 mesh silica gel, Petroleum ether/Ethyl acetate=100/0, 7/93). 2 (600 mg, 2.05 mmol, 63.25% yield) as a white solid.

LCMS: RT=0.512 min, MS cal.: 293.1, [M+H−t-Bu]⁺=238.1

HPLC: product: RT=3.814 min, purity: 89.33%

¹H NMR (400 MHz, MeOH-d4) δ=7.04 (dt, J=6.4, 8.0 Hz, 1H), 6.60 (d, J=8.0 Hz, 1H), 6.57-6.52 (m, 1H), 5.66 (br s, 1H), 4.03 (br s, 2H), 3.62 (br s, 2H), 2.37 (br d, J=2.0 Hz, 2H), 1.50 (s, 9H)

### General Procedure for Preparation of Intermediate 3:

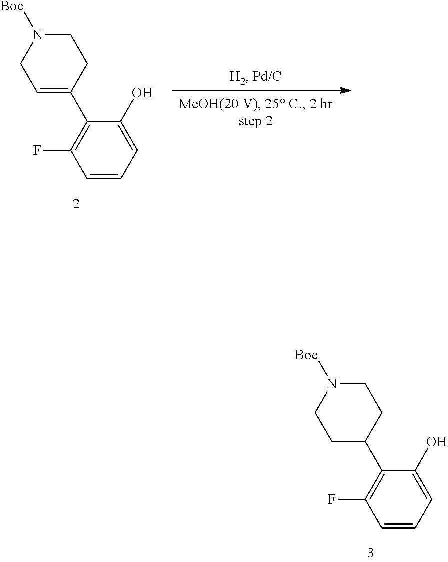 [SMILES: CC(C)(C)OC(=O)N1CC=C(c2c(O)cccc2F)CC1 ; "H2, Pd/C" ; "MeOH(20 V), 25° C., 2 hrstep 2" ; CC(C)(C)OC(=O)N1CCC(c2c(O)cccc2F)CC1]

To a mixture of 2 (300 mg, 1.02 mmol, 1 eq) in MeOH (6 mL) was added Pd/C (108.84 mg, 102.27 μmol, 10% purity, 0.1 eq) in one portion at 25° C. under N₂. The mixture was de-gassed under reduced pressure and recharged with N₂. Then, the mixture was de-gassed under reduced pressure and recharged with H₂ (15 Psi). The mixture was stirred at 25° C. for 2 hours under H₂ (15 Psi). LC-MS showed 2 was consumed completely and one main peak with desired mass was detected. The suspension was filtered, and the filter cake was washed with MeOH (5 mL*3). The combined filtrates were concentrated to dryness to give product. 3 (280 mg, 948.03 μmol, 92.70% yield) as a white solid.

LCMS: RT=0.535 min, MS cal.: 295.2, [M+H-t-Bu]⁺=240.1

HPLC: product: RT=3.906 min, purity: 97.64%

¹H NMR (400 MHz, MeOH-d4) δ=6.96 (dt, J=6.4, 8.0 Hz, 1H), 6.56 (d, J=8.0 Hz, 1H), 6.49 (ddd, J=1.2, 8.4, 11.2 Hz, 1H), 4.17 (br d, J=13.2 Hz, 2H), 3.30-3.24 (m, 1H), 2.81 (br d, J=1.2 Hz, 2H), 2.18-2.04 (m, 2H), 1.58 (br d, J=12.8 Hz, 2H), 1.48 (s, 9H)

### General Procedure for Preparation of Intermediate 4:

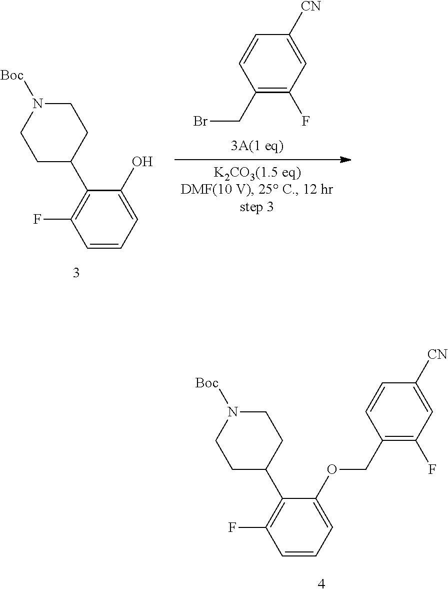

To a mixture of 3 (200 mg, 677.17 μmol, 1 eq) and 3A (144.94 mg, 677.17 μmol, 1 eq) in DMF (2 mL) was added K₂CO₃ (140.39 mg, 1.02 mmol, 1.5 eq) in one portion at 25° C. The mixture was stirred at 25° C. for 2 hours. LC-MS showed Compound 3 was consumed completely and one main peak with desired mass was detected. The suspension was filtered, the mixture was concentrated in vacuum. The crude product was purified by prep-TLC (Petroleum ether:Ethyl acetate=5:1, Rf=0.45). 4 (220 mg, 513.45 μmol, 75.82% yield) as a white solid.

LCMS: RT=0.639 min, MS cal.: 428.2, [M−t-Bu+ACN]⁺=414.2

HPLC: product: RT=5.477 min, purity: 89.40%

¹H NMR (400 MHz, MeOH-d4) δ=7.56 (t, J=8.0 Hz, 1H), 7.50 (dd, J=1.6, 10.0 Hz, 1H), 7.33 (dd, J=1.2, 8.0 Hz, 1H), 7.25-7.18 (m, 1H), 6.97 (d, J=8.4 Hz, 1H), 6.82-6.75 (m, 1H), 5.16 (s, 2H), 4.01 (br d, J=6.4 Hz, 2H), 3.24-3.14 (m, 1H), 2.79-2.61 (m, 2H), 1.95-1.81 (m, 2H), 1.47 (br d, J=11.2 Hz, 2H), 1.36 (s, 9H)

### General Procedure for Preparation of Intermediate 5:

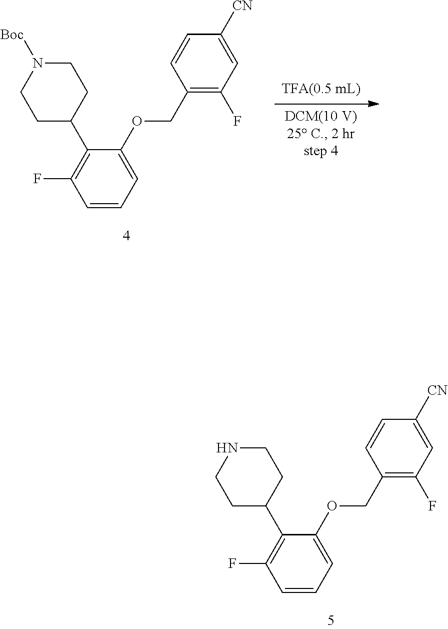 [SMILES: CC(C)(C)OC(=O)N1CCC(c2c(F)cccc2OCc2ccc(C#N)cc2F)CC1 ; "DCM(10 V)25° C., 2 hrstep 4" ; "TFA(0.5 mL)" ; N#Cc1ccc(COc2cccc(F)c2C2CCNCC2)c(F)c1]

To a mixture of 4 (200 mg, 466.78 μmol, 1 eq) in DCM (2 mL) was added TFA (767.48 mg, 6.73 mmol, 499.99 μL, 14.42 eq) in one portion at 25° C. The mixture was stirred at 25° C. for 1 hour. LC-MS showed 4 was consumed completely and one main peak with desired mass was detected. To a mixture was added Saturated sodium carbonate solution to PH=9˜10. The aqueous phase was extracted with DCM (2 mL*3). The combined organic phase was washed with brine (3 mL*2), dried with anhydrous Na₂SO₄, filtered and concentrated in vacuum. The crude product was purified by prep-TLC (Petroleum ether:Ethyl acetate=1:1, Rf=0). 5 (130 mg, 395.91 μmol, 84.82% yield) as a yellow solid.

LCMS: RT=0.376 min, MS cal.: 328.1, [M+H]⁺=329.2

HPLC: product: RT=2.703 min, purity: 93.34%

¹H NMR (400 MHz, CHCl3-d) δ=7.51 (t, J=8.0 Hz, 1H), 7.31-7.24 (m, 2H), 7.24-7.19 (m, 1H), 6.92 (d, J=8.4 Hz, 1H), 6.74 (dd, J=8.4, 11.2 Hz, 1H), 5.18 (s, 2H), 3.52-3.42 (m, 3H), 3.06 (dt, J=2.0, 13.2 Hz, 2H), 2.35 (dq, J=4.0, 13.6 Hz, 2H), 1.85 (br d, J=14.0 Hz, 2H)

### General Procedure for Preparation of Intermediate 6:

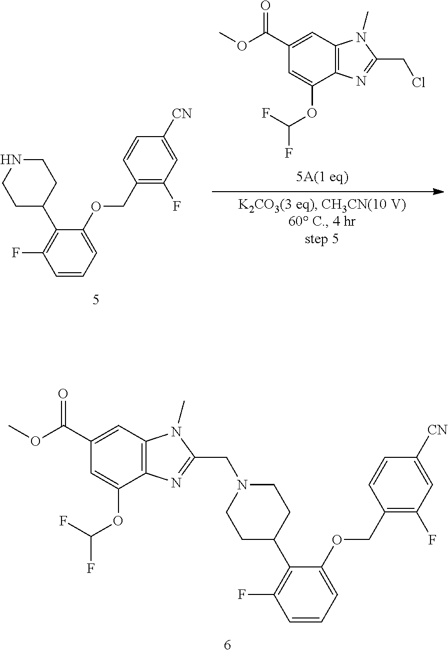

To a mixture of 5 (90 mg, 274.09 μmol, 1 eq) and 5A (83.51 mg, 274.09 μmol, 1 eq) in ACN (2 mL) was added K₂CO₃ (113.64 mg, 822.28 μmol, 3 eq) in one portion at 25° C. The mixture was stirred at 60° C. for 4 hours. LC-MS (ET83209-16-P1B7) showed 5 was consumed completely and one main peak with desired mass was detected. The mixture was filtered and concentrated in vacuum. The residue was purified by prep-TLC (SiO₂, Petroleum ether:Ethyl acetate=3:1, R_{f}=0.3). 6 (70 mg, 111.47 μmol, 40.67% yield, 95% purity) as a yellow solid.

LCMS: RT=0.934 min, MS cal.: 596.2, [M+H]⁺=597.4

HPLC: product: RT=3.565 min, purity: 95.06%

¹H NMR (400 MHz, CHCl3-d) δ=8.12 (d, J=1.2 Hz, 1H), 7.99-7.94 (m, 1H), 7.61 (t, J=72 Hz, 1H), 7.79-7.73 (m, 2H), 7.53 (s, 1H), 7.24-7.17 (m, 1H), 6.96 (d, J=8.4 Hz, 1H), 6.78 (dd, J=8.8, 10.8 Hz, 1H), 5.27 (s, 2H), 3.90 (d, J=1.6 Hz, 6H), 3.86-3.80 (m, 2H), 3.10-3.01 (m, 1H), 2.94-2.84 (m, 2H), 2.17-1.99 (m, 4H), 1.50 (br d, J=10.4 Hz, 2H)

### General Procedure for Preparation of Compound 146 (7):

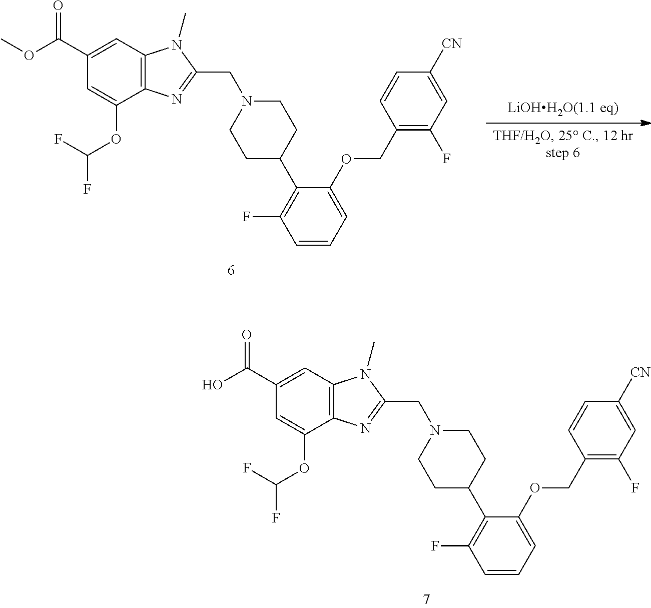 [SMILES: COC(=O)c1cc(OC(F)F)c2nc(CN3CCC(c4c(F)cccc4OCc4ccc(C#N)cc4F)CC3)n(C)c2c1 ; "LiOH·H2O(1.1 eq)" ; "THF/H2O, 25° C., 12 hrstep 6" ; Cn1c(CN2CCC(c3c(F)cccc3OCc3ccc(C#N)cc3F)CC2)nc2c(OC(F)F)cc(C(=O)O)cc21]

To a mixture of 6 (70 mg, 117.34 μmol, 1 eq) in THF (0.3 mL) and H₂O (0.7 mL) was added LiOH·H₂O (5.42 mg, 129.07 μmol, 1.1 eq) in one portion at 25° C. LC-MS showed 6 was consumed completely and one main peak with desired mass was detected. The mixture was filtered and concentrated in vacuum. The crude product was purified by prep-HPLC (column: Waters Xbridge Prep OBD C₁₈ 150*40 mm*10 um; mobile phase: [H₂O (10 mM NH₄HCO₃)-ACN]; gradient: 25%-55% B over 8.0 min). 2-((4-(2-((4-Cyano-2-fluorobenzyl)oxy)-6-fluorophenyl)piperidin-1-yl)methyl)-4-(difluoromethoxy)-1-methyl-1H-benzo[d]imidazole-6-carboxylic acid (25.24 mg, 43.33 μmol, 36.93% yield, 100% purity) as a white solid.

LCMS: RT=2.701 min, MS cal.: 582.2, [M+H]⁺=583.2

HPLC: RT=11.173 min, purity: 100%

¹H NMR (400 MHz, MeOH-d4) δ=8.10 (s, 1H), 7.75-7.70 (m, 1H), 7.70-7.62 (m, 3H), 7.21 (t, J=72 Hz, 1H), 7.20-7.13 (m, 1H), 6.86 (d, J=8.4 Hz, 1H), 6.70 (dd, J=8.4, 10.4 Hz, 1H), 5.25 (s, 2H), 3.97 (s, 3H), 3.91 (s, 2H), 3.25-3.16 (m, 1H), 3.06-2.97 (m, 2H), 2.35-2.21 (m, 4H), 1.63-1.57 (m, 2H)

### Example A190—Preparation of Compound 378. (S)-2-((4-(6-((4-Cyano-2-fluorobenzyl)oxy)pyridin-2-yl)piperidin-1-yl)methyl)-1-(oxetan-2-ylmethyl)-1H-imidazo[4,5-c]pyridine-6-carboxylic acid (Compound 378) (6A)

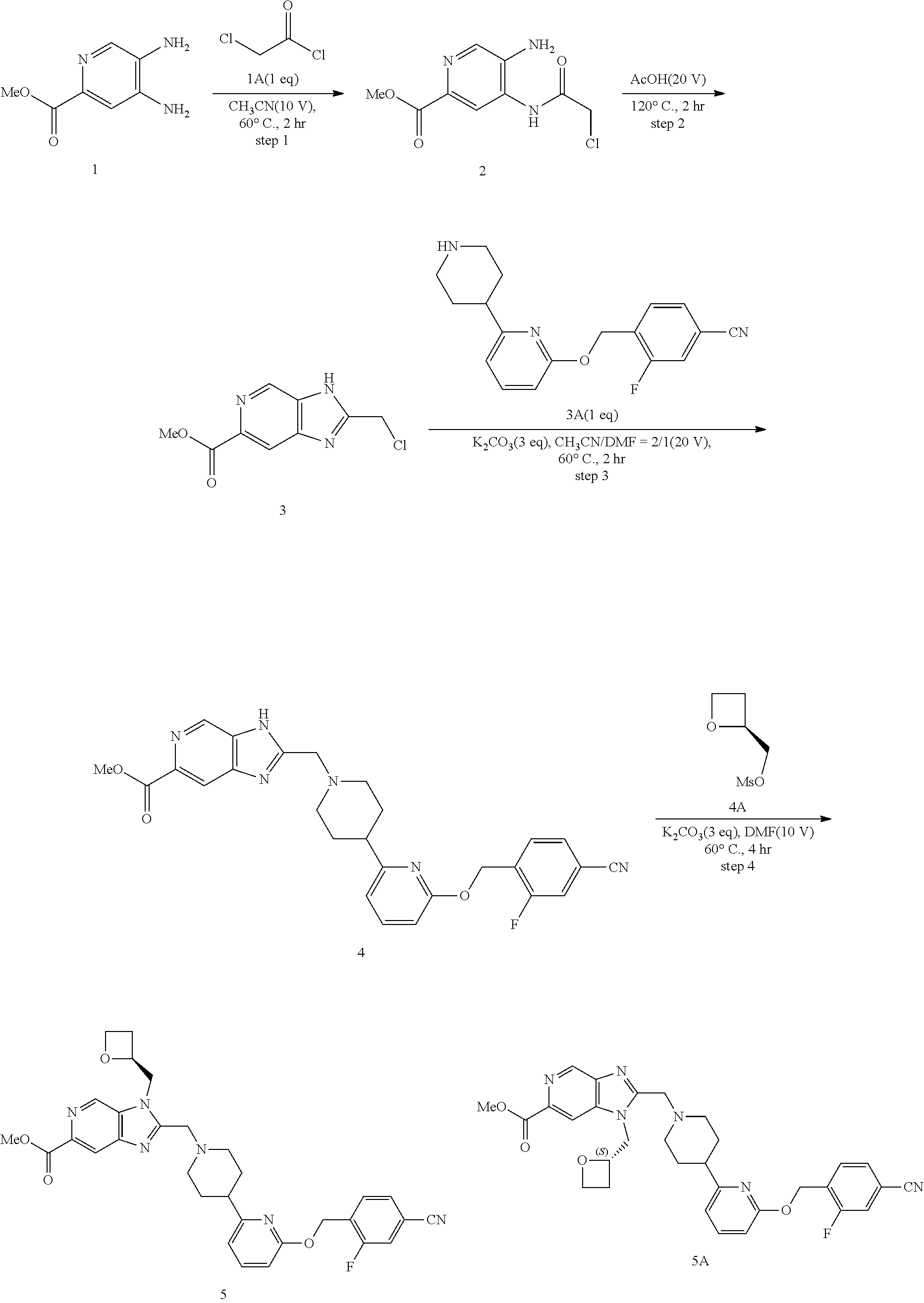

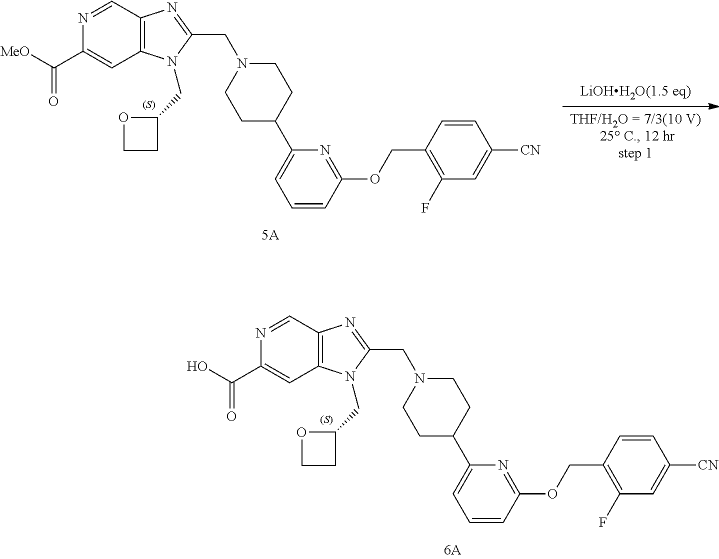

### General Procedure for Preparation of Intermediate 2:

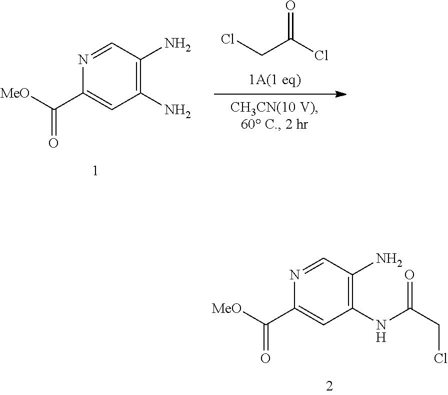

The reactions were carried out in parallel as two batch. To a solution of 1 (400 mg, 2.39 mmol, 1 eq) in ACN (5 mL) was added 1A (270.25 mg, 2.39 mmol, 190.59 μL, 1 eq). The mixture was stirred at 60° C. for 1 hr. LC-MS showed desired mass was detected. 2 reactions were combined for workup. The reaction mixture was concentrated under reduced pressure to remove solvent. 2 (1.1 g, crude) was obtained as yellow oil.

LCMS: RT=0.665 min, MS cal.: 243.0, [M+H]⁺=244.0

### General Procedure for Preparation of Intermediate 3:

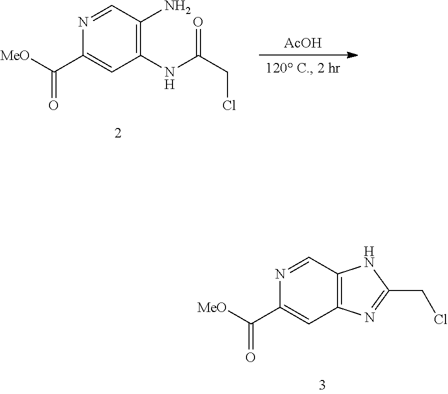 [SMILES: "AcOH" ; COC(=O)c1cc(NC(=O)CCl)c(N)cn1 ; "120° C., 2 hr" ; COC(=O)c1cc2nc(CCl)[nH]c2cn1]

Three reactions were performed in parallel. A solution of 2 (200 mg, 820.86 μmol, 1 eq) in AcOH (5 mL) was stirred at 120° C. for 2 hr. LC-MS showed 2 was consumed completely and desired mass was detected. 2 reactions were combined for workup. The reaction mixture was concentrated under reduced pressure to remove solvent. 3 (185 mg, crude) was obtained as yellow oil.

LCMS: RT=0.847 min, MS cal.: 225.0, [M+H]⁺=226.0

### General Procedure for Preparation of Intermediate 4:

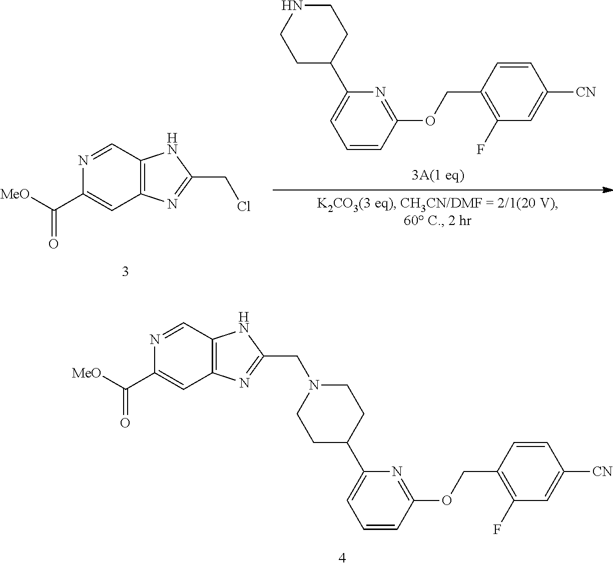

To a solution of 3 (185 mg, 409.96 μmol, 1 eq) in ACN (4 mL) and DMF (2 mL) was added K₂CO₃ (169.98 mg, 1.23 mmol, 3 eq) and 3A (114.88 mg, 368.96 μmol, 0.9 eq). The mixture was stirred at 60° C. for 2 hr. LC-MS showed 3 was consumed completely and desired mass was detected. The residue was diluted with H₂O 15 mL and extracted with EtOAc 45 mL (15 mL*3). The combined organic layers were washed with brine 15 mL, dried over Na₂SO₄, filtered, and concentrated under reduced pressure to give a residue. The residue was purified by column chromatography (SiO₂, DCM/MeOH=100/1 to 5/1) (Rf=0.53). 4 (120 mg, 239.75 μmol, 58.48% yield) was obtained as a yellow oil.

LCMS: RT=1.793 min, MS cal.: 500.2, [M+H]⁺=501.2

### General Procedure for Preparation of Intermediate 5+5A:

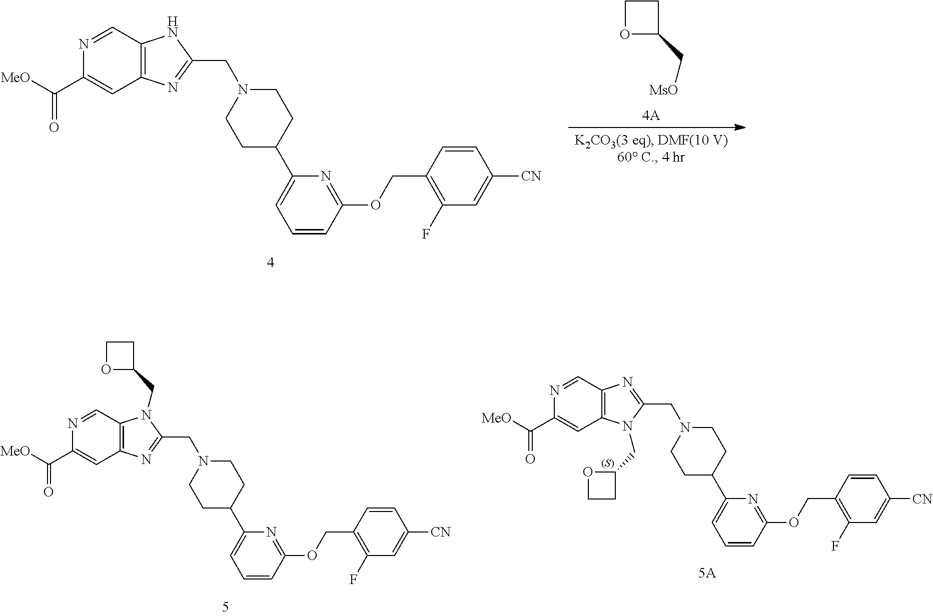

Two reactions were performed in parallel. To a solution of 4 (100 mg, 199.79 μmol, 1 eq) in

DMF (1 mL) was added K₂CO₃ (82.84 mg, 599.37 μmol, 3 eq), NaI (29.95 mg, 199.79 μmol, 1 eq) and 4A (166.02 mg, 998.95 μmol, 5 eq). The mixture was stirred at 60° C. for 12 hr. LC-MS showed 4 was consumed completely and desired mass was detected. 2 reactions were combined for workup. The residue was diluted with H₂O 20 mL and extracted with EtOAc 45 mL (15 mL*3). The combined organic layers were washed with brine 15 mL, dried over Na₂SO₄, filtered and concentrated under reduced pressure to give a residue. The residue was purified by column chromatography (SiO₂, Ethyl acetate/MeOH=95/1 to 5/1) to give 5 & 5A (200 mg, purity 93%) as yellow oil, which was further separated by SFC (condition: column: REGIS (S,S) WHELK-O1 (250 mm*25 mm, 10 um); mobile phase: [CO₂-EtOH (0.1% NH₃H₂O)]; B %: 70%, isocratic elution mode). 5A (100 mg, 175.25 μmol, 43.86% yield) was obtained as colorless oil. (peak 1)

The peak 2 was purified by prep-HPLC (neutral condition; column: Phenomenex Gemini-NX 80*30 mm*3 um; mobile phase: [H₂O (10 mM NH₄HCO₃)-ACN]; gradient: 40%-70% B over 8.0 min) to give 5 (50 mg, 87.63 μmol, 21.93% yield) as a white solid.

LCMS: RT=2.007 min, MS cal.: 570.2, [M+H]⁺=571.2

¹H NMR (400 MHz, MeOH-d4) δ=9.10 (s, 1H), 8.45 (s, 1H), 7.73-7.65 (m, 1H), 7.64-7.52 (m, 3H), 6.86 (d, J=7.2 Hz, 1H), 6.71 (d, J=8.2 Hz, 1H), 5.62-5.49 (m, 2H), 5.37-5.22 (m, 1H), 5.03-4.97 (m, 1H), 4.85-4.80 (m, 1H), 4.73-4.62 (m, 1H), 4.60-4.45 (m, 1H), 4.09-4.05 (m, 1H), 4.02 (s, 3H), 3.97-3.90 (m, 1H), 3.07 (br d, J=12.0 Hz, 1H), 2.96-2.79 (m, 2H), 2.71-2.50 (m, 2H), 2.42-2.21 (m, 2H), 1.92-1.73 (m, 4H)

¹H NMR (400 MHz, MeOH-d4) δ=8.94 (s, 1H), 8.57-8.54 (m, 1H), 7.70-7.64 (m, 1H), 7.62-7.51 (m, 3H), 6.84 (d, J=7.2 Hz, 1H), 6.68 (d, J=8.2 Hz, 1H), 5.52 (s, 2H), 5.28 (dq, J=2.4, 7.2 Hz, 1H), 4.81-4.75 (m, 1H), 4.69-4.62 (m, 1H), 4.49 (td, J=6.0, 9.2 Hz, 1H), 4.09-4.03 (m, 1H), 4.00 (s, 3H), 3.95-3.87 (m, 1H), 3.04 (br d, J=11.6 Hz, 1H), 2.91 (br d, J=11.6 Hz, 1H), 2.87-2.77 (m, 1H), 2.69-2.48 (m, 2H), 2.38-2.22 (m, 2H), 1.87-1.76 (m, 4H)

### General Procedure for Preparation of Compound 378 (6A):

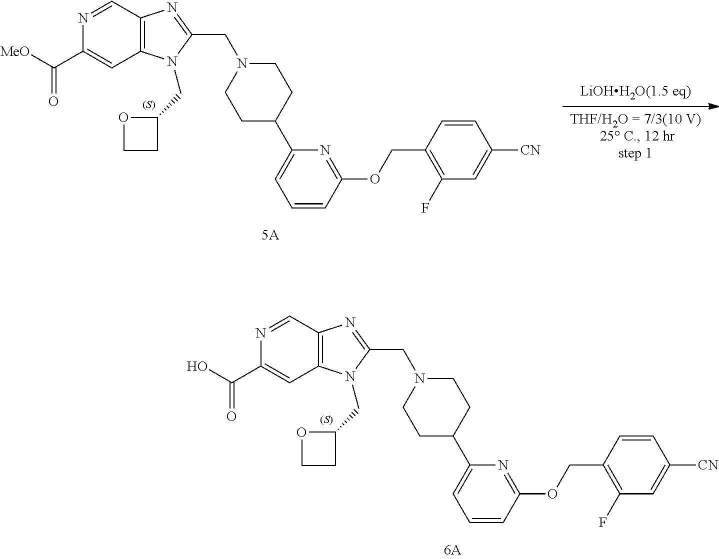

A mixture of 5A (70 mg, 122.68 μmol, 1 eq), LiOH·H₂O (6.18 mg, 147.21 μmol, 1.2 eq) in THF (0.7 mL) and H₂O (0.3 mL) was degassed and purged with N₂ for 3 times, and then the mixture was stirred at 25° C. for 12 hr under N₂ atmosphere. LCMS (Product, RT=1.468 min) showed 88% of desired MS was detected. The reaction was concentrated. The residue was purified by prep-HPLC (neutral condition column: Waters Xbridge Prep OBD C18 150*40 mm*10 um; mobile phase: [H₂O (10 mM NH₄HCO₃)-ACN]; gradient: 25%-55% B over 8.0 min). (S)-2-((4-(6-((4-Cyano-2-fluorobenzyl)oxy)pyridin-2-yl)piperidin-1-yl)methyl)-1-(oxetan-2-ylmethyl)-1H-imidazo[4,5-c]pyridine-6-carboxylic acid (28.19 mg, 50.14 μmol, 40.87% yield, 99% purity) was obtained as a white solid.

LCMS: RT=1.468 min, MS cal.: 556.6, [M+H]⁺=557.2

LCMS: RT=2.559 min, MS cal.: 556.6, [M+H]⁺=557.2

HPLC: RT=8.143 min, purity: 99%

1H NMR (400 MHz, DMSO-d6) δ=8.93 (s, 1H), 8.44 (s, 1H), 7.88 (d, J=10.4 Hz, 1H), 7.73-7.68 (m, 2H), 7.67-7.62 (m, 1H), 6.88 (d, J=7.2 Hz, 1H), 6.72 (d, J=8.0 Hz, 1H), 5.47 (s, 2H), 5.12 (dq, J=2.4, 7.1 Hz, 1H), 4.86 (dd, J=7.2, 15.1 Hz, 1H), 4.74-4.66 (m, 1H), 4.52-4.44 (m, 1H), 4.38 (td, J=6.0, 9.0 Hz, 1H), 3.98 (d, J=13.6 Hz, 1H), 3.83 (br d, J=13.6 Hz, 1H), 2.98 (br d, J=11.2 Hz, 1H), 2.84 (br d, J=11.2 Hz, 1H), 2.74-2.67 (m, 1H), 2.62-2.56 (m, 1H), 2.46-2.38 (m, 1H), 2.21 (dq, J=9.6, 11.6 Hz, 2H), 1.80-1.61 (m, 4H)

### Example A191—Preparation of Compound 147. 2-((4-(6-((4-Cyano-2-fluorobenzyl)oxy)pyridin-2-yl)piperidin-1-yl)methyl)-1-(difluoromethyl)-4-methoxy-1H-benzo[d]imidazole-6-carboxylic acid (Compound 147) (9)

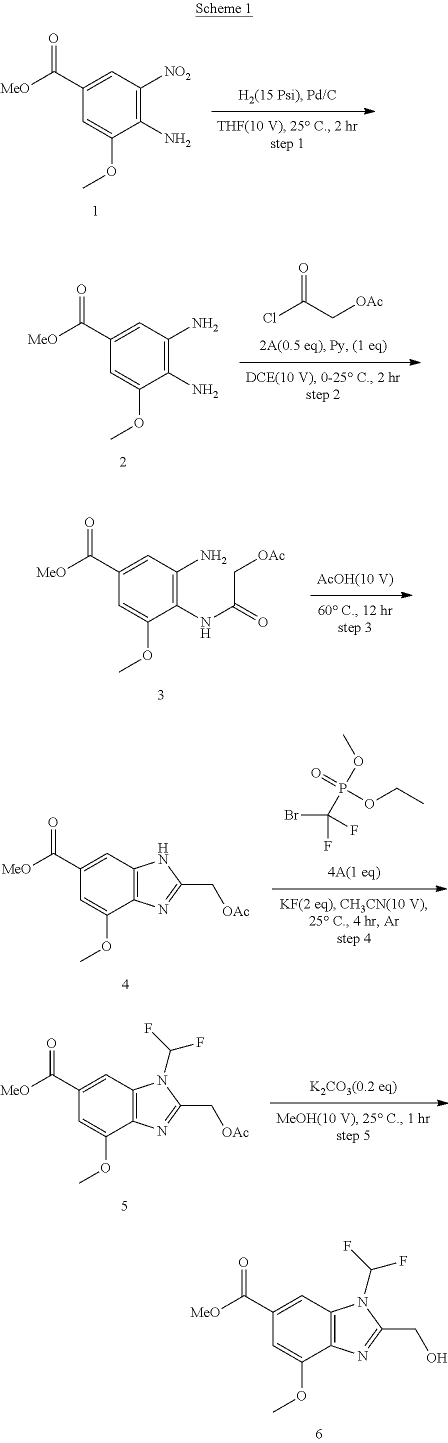

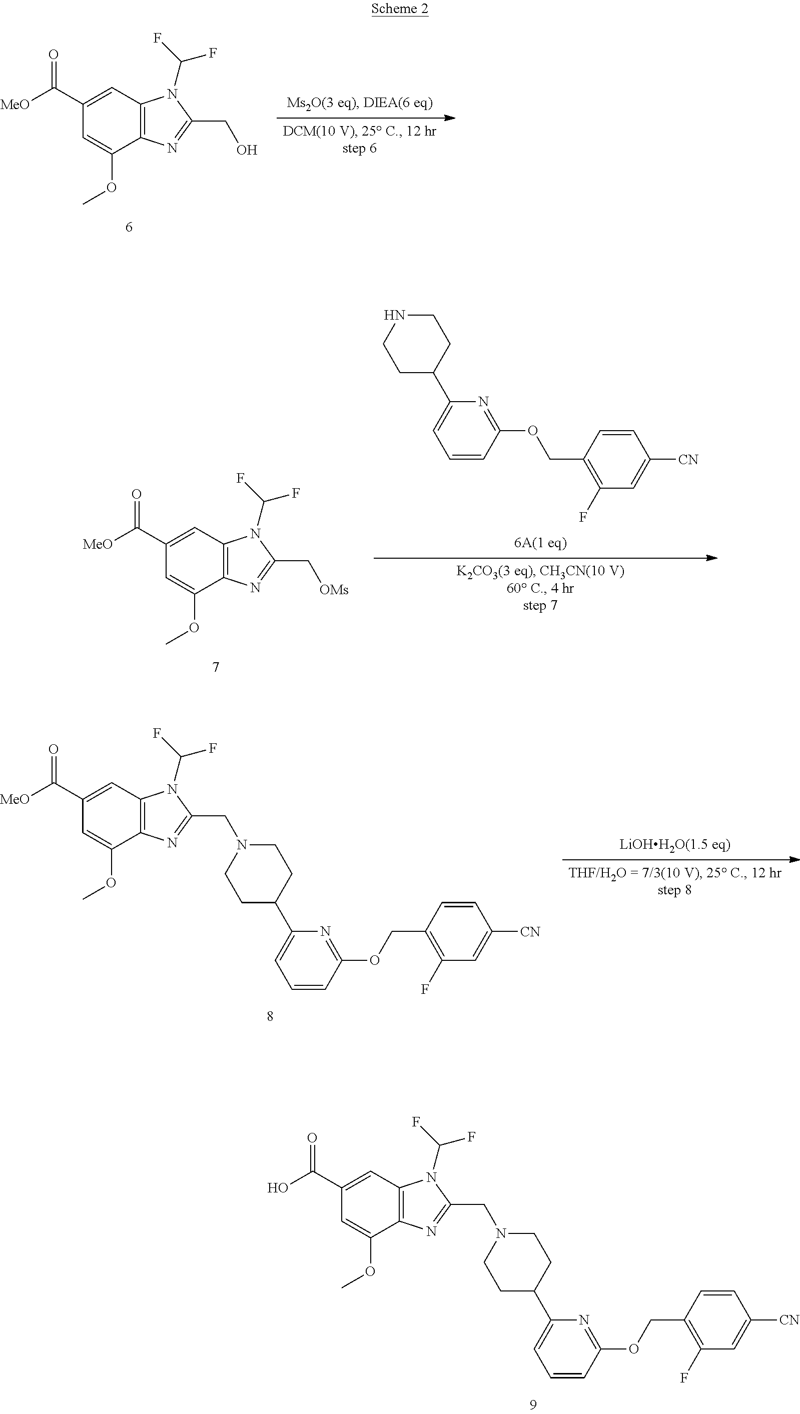

### General Procedure for Preparation of Intermediate 2:

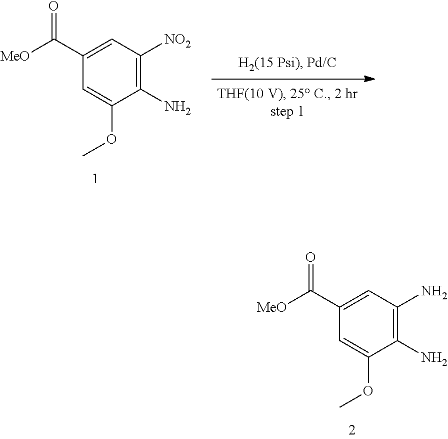 [SMILES: COC(=O)c1cc(OC)c(N)c([N+](=O)[O-])c1 ; COC(=O)c1cc(N)c(N)c(OC)c1]

THF (30 mL) was charged to the three-necked round bottom flask, then starting 1 (2 g, 8.84 mmol, 1 eq) and was added to the mixture at 25° C. At 25° C., Pd/C (500 mg, 10% purity) was added in portions to the reaction mixture at 25° C. After the addition, the mixture was stirred at 25° C. for 2 hr under H₂ (15 Psi) atmosphere. LCMS showed 1 was consumed completely and one main peak with desired mass was detected. The reaction mixture was filtered and concentrated under reduced pressure to give a residue. 2 (1.69 g, 8.61 mmol, 97.41% yield) was obtained as a yellow solid.

LCMS: RT=0.638 min, MS cal.: 196.1, [M+H]⁺=197.1

¹H NMR (400 MHz, CHCl3-d) δ=7.17 (d, J=1.6 Hz, 1H), 7.15 (d, J=1.6 Hz, 1H), 3.90 (s, 3H), 3.87 (s, 3H)

### General Procedure for Preparation of Intermediate 3:

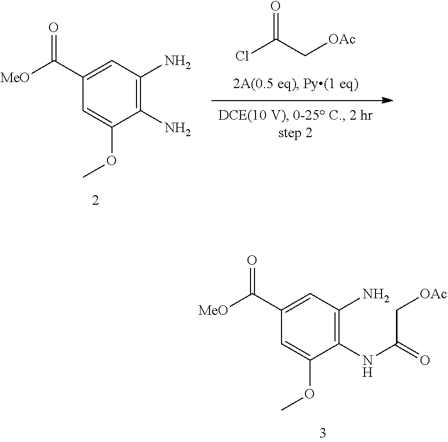 [SMILES: COC(=O)c1cc(N)c(N)c(OC)c1 ; CC(=O)OCC(=O)Cl ; COC(=O)c1cc(N)c(NC(=O)COC(C)=O)c(OC)c1]

DCE (5 mL) was charged to the three-necked round bottom flask, then 2 (500 mg, 2.55 mmol, 1 eq) and Py. (201.58 mg, 2.55 mmol, 205.69 μL, 1 eq) was added to the mixture at 0° C. At 0° C., 2A (173.97 mg, 1.27 mmol, 136.98 μL, 0.5 eq) was added dropwise to the reaction mixture at 0° C. After the addition, the mixture was stirred at 25° C. for 3 hr. LCMS showed ˜34% of 2 remained. Several new peaks were shown on LC-MS and ˜36% of 3 was detected. The reaction mixture was added to H₂O (10 mL) at 25° C. The mixture was extracted by DCM (10 mL*3). Then organic phase was combined and washed by H₂O (5 mL), brine (10 mL), dried with Na₂SO₄, filtered. The organic phase was concentrated under reduced pressure at 35° C. to give a residue. 3 (300 mg, crude) was obtained as a yellow solid.

LCMS: RT=0.949 min, MS cal.: 296.1, [M+H]⁺=297.1

### General Procedure for Preparation of Intermediate 4:

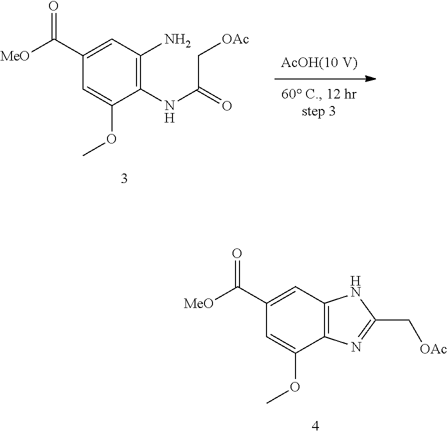 [SMILES: COC(=O)c1cc(N)c(NC(=O)COC(C)=O)c(OC)c1 ; COC(=O)c1cc(OC)c2nc(COC(C)=O)[nH]c2c1]

A mixture of 3 (300 mg, 1.01 mmol, 1 eq) in AcOH (3 mL) was degassed and purged with N₂ for 3 times, and then the mixture was stirred at 60° C. for 12 hr under N₂ atmosphere. LCMS showed 3 was consumed completely and one main peak with desired mass was detected. The mixture was added with 2M aq. Na₂CO₃ to adjust pH=7 and extracted with EtOAc (15 mL*3). The combined organic layers were washed with brine (15 mL*3), dried over Na₂SO₄, filtered, and concentrated under reduced pressure to give a residue. The residue was purified by column chromatography (SiO₂, Petroleum ether/Ethyl acetate=10/1 to 0/1). 4 (200 mg, 718.75 μmol, 70.98% yield) was obtained as a yellow solid.

LCMS: RT=0.312 min, MS cal.: 278.1, [M+H]⁺=279.0

### General Procedure for Preparation of Intermediate 5:

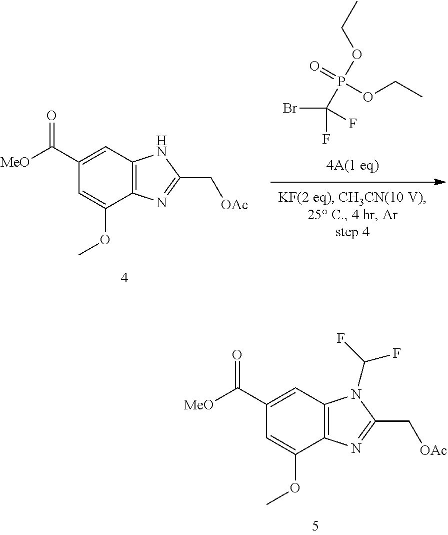 [SMILES: COC(=O)c1cc(OC)c2nc(COC(C)=O)[nH]c2c1 ; CCOP(=O)(OCC)C(F)(F)Br ; COC(=O)c1cc(OC)c2nc(COC(C)=O)n(C(F)F)c2c1]

A mixture of 4 (200 mg, 718.75 μmol, 1 eq), 4A (191.91 mg, 718.75 μmol, 1 eq), KF (83.51 mg, 1.44 mmol, 2 eq) in MeCN (1 mL) was degassed and purged with Ar for 3 times, and then the mixture was stirred at 25° C. for 4 hr under Ar atmosphere. LCMS showed 4 was consumed completely and one main peak with desired mass was detected. The reaction mixture was filtered and concentrated under reduced pressure to give a residue. The residue was purified by column chromatography (SiO₂, Petroleum ether/Ethyl acetate=10/1 to 0/1). 5 (100 mg, 286.35 μmol, 39.84% yield, 94% purity) was obtained as a yellow oil.

LCMS: RT=0.424 min, MS cal.: 328.1, [M+H]⁺=329.0

¹H NMR (400 MHz, CHCl3-d) δ=8.02 (s, 1H), 7.69 (s, 1H), 7.54 (t, J=58.8 Hz, 1H), 5.45 (s, 2H), 4.09 (s, 3H), 3.98 (s, 3H), 2.16 (s, 3H)

### General Procedure for Preparation of Intermediate 6:

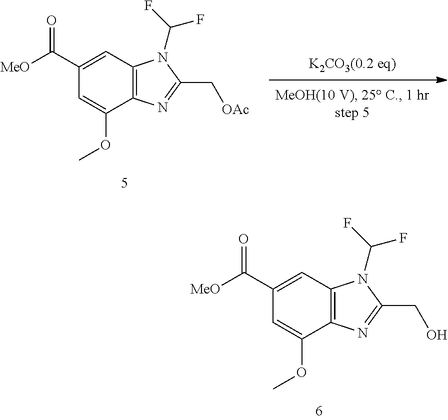 [SMILES: COC(=O)c1cc(OC)c2nc(COC(C)=O)n(C(F)F)c2c1 ; COC(=O)c1cc(OC)c2nc(CO)n(C(F)F)c2c1]

A mixture of 5 (100 mg, 304.63 μmol, 1 eq), K₂CO₃ (8.42 mg, 60.93 μmol, 0.2 eq) in MeOH (1 mL) was degassed and purged with N₂ for 3 times, and then the mixture was stirred at 25° C. for 2 hr under N₂ atmosphere. LCMS showed 6 was consumed completely and one main peak with desired mass was detected. The reaction mixture was filtered and concentrated under reduced pressure to give a residue. 6 (75 mg, 262.03 μmol, 86.01% yield) was obtained as a yellow solid.

LCMS: RT=0.364 min, MS cal.: 286.1, [M+H]⁺=287.0

¹H NMR (400 MHz, CHCl3-d) δ=8.00 (s, 1H), 7.69 (t, J=58.8 Hz, 1H)), 7.50 (s, 1H), 5.06 (br s, 2H), 4.08 (s, 3H), 3.97 (s, 3H)

### General Procedure for Preparation of Intermediate 7:

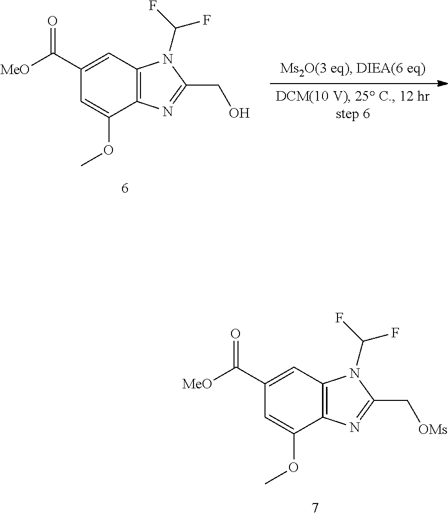 [SMILES: COC(=O)c1cc(OC)c2nc(CO)n(C(F)F)c2c1 ; "DCM(10 V), 25° C., 12 hrstep 6" ; "Ms2O(3 eq), DIEA(6 eq)" ; COC(=O)c1cc(OC)c2nc(COS(C)(=O)=O)n(C(F)F)c2c1]

A mixture of 6 (75 mg, 262.03 μmol, 1 eq), Ms₂O (136.93 mg, 786.08 μmol, 3 eq), DIEA (203.19 mg, 1.57 mmol, 273.84 μL, 6 eq) in DCM (1 mL) was degassed and purged with N₂ for 3 times, and then the mixture was stirred at 25° C. for 0.5 hr under N₂ atmosphere. LCMS showed 6 was consumed completely and one main peak with desired mass was detected. The reaction mixture was filtered and concentrated under reduced pressure to give a residue. 7 (90 mg, 247.03 μmol, 94.28% yield) was obtained as a white solid.

LCMS: RT=0.412 min, MS cal.: 364.1, [M+H]⁺=365

### General Procedure for Preparation of Intermediate 8:

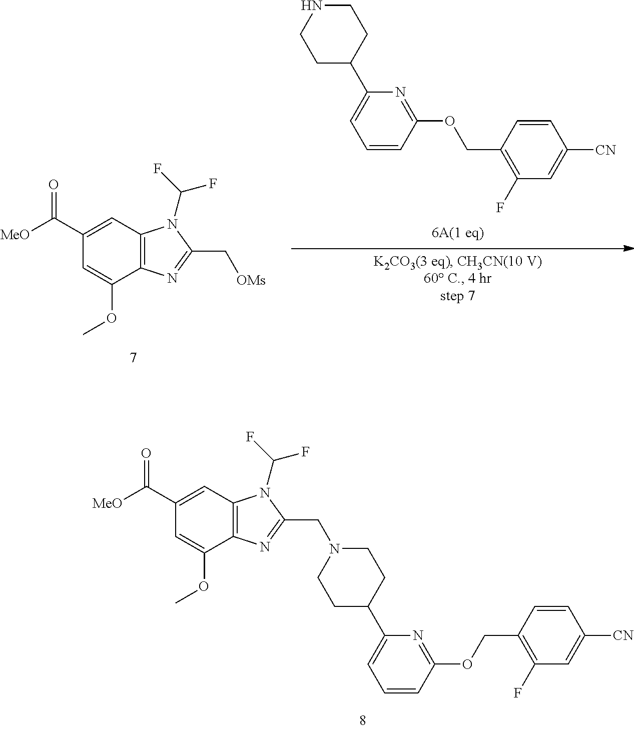 [SMILES: COC(=O)c1cc(OC)c2nc(COS(C)(=O)=O)n(C(F)F)c2c1 ; N#Cc1ccc(COc2cccc(C3CCNCC3)n2)c(F)c1 ; "6A(1 eq)" ; "K2CO3(3 eq), CH3CN(10 V)60° C., 4 hrstep 7" ; COC(=O)c1cc(OC)c2nc(CN3CCC(c4cccc(OCc5ccc(C#N)cc5F)n4)CC3)n(C(F)F)c2c1]

A mixture of 7 (80 mg, 219.59 μmol, 1 eq), 6 A (68.37 mg, 219.59 μmol, 1 eq), K₂CO₃ (91.04 mg, 658.76 μmol, 3 eq) in CH₃CN (1 mL) was degassed and purged with N₂ for 3 times, and then the mixture was stirred at 60° C. for 2 hr under N₂ atmosphere. LCMS showed 7 was consumed completely and one main peak with desired mass was detected. The combined mixture was diluted with H₂O (10 mL) and extracted with EtOAc (10 mL*3). The combined organic layers were washed with brine (10 mL*3), dried over Na₂SO₄, filtered, and concentrated under reduced pressure to give a residue. The residue was purified by column chromatography (SiO₂, Petroleum ether/Ethyl acetate=10/1 to 0/1). 8 (90 mg, 155.29 μmol, 70.72% yield) was obtained as a yellow solid.

LCMS: RT=0.470 min, MS cal.: 579.2, [M+H]⁺=580.3

¹H NMR (400 MHz, DMSO-d6) δ=8.29 (t, J=58 Hz, 1H)), 7.89-7.84 (m, 2H), 7.69 (s, 2H), 7.66-7.61 (m, 1H), 7.42-7.39 (m, 1H), 6.88-6.85 (m, 1H), 6.73-6.69 (m, 1H), 5.45 (s, 2H), 3.99 (s, 3H), 3.96 (s, 2H), 3.90 (s, 3H), 2.88-2.81 (m, 2H), 2.62-2.55 (m, 1H), 2.21-2.11 (m, 2H), 1.78-1.61 (m, 4H)

### General Procedure for Preparation of Compound 147 (9):

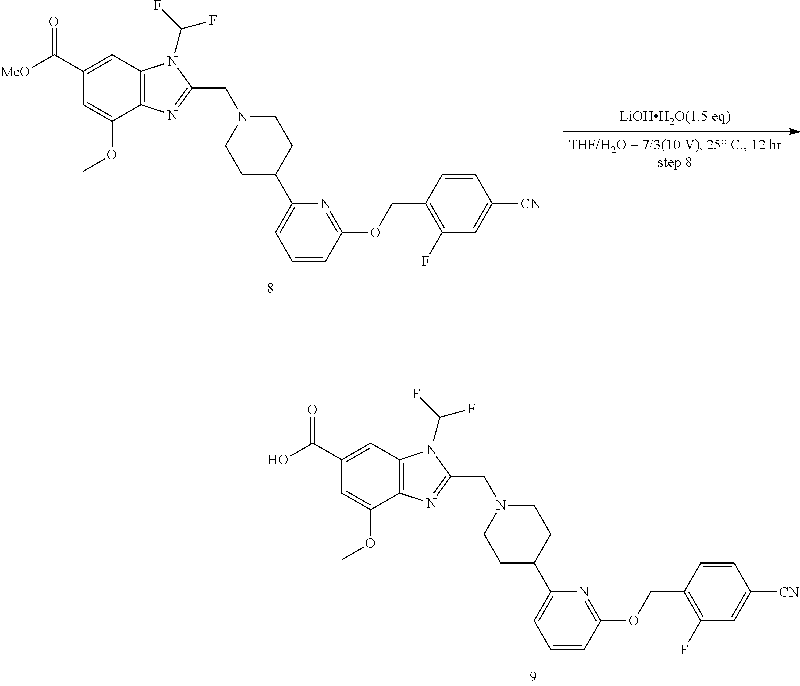

A mixture of 8 (80 mg, 138.03 μmol, 1 eq), LiOH·H₂O (8.69 mg, 207.05 μmol, 1.5 eq) in THF (0.7 mL) and H₂O (0.3 mL) was degassed and purged with N₂ for 3 times, and then the mixture was stirred at 25° C. for 12 hr under N₂ atmosphere. LCMS showed 8 was consumed completely and one main peak with desired mass was detected. The combined mixture was added with 0.5M citric acid to adjust pH=7. The mixture was purified by prep-HPLC (column: Waters Xbridge Prep OBD C18 150*40 mm*10 um; mobile phase: [H₂O (10 mM NH₄HCO₃)-ACN]; gradient:

20%-55% B over 8.0 min). 2-((4-(6-((4-Cyano-2-fluorobenzyl)oxy)pyridin-2-yl)piperidin-1-yl)methyl)-1-(difluoromethyl)-4-methoxy-1H-benzo[d]imidazole-6-carboxylic acid (25.36 mg, 44.77 μmol, 32.44% yield, 99.85% purity) was obtained as a white solid.

LCMS: RT=2.667 min, MS cal.: 565.2, [M+H]⁺=566.2

HPLC: RT=10.707 min, purity: 99.85%

¹H NMR (400 MHz, DMSO-d6) δ=8.27 (t, J=58 Hz, 1H)), 7.89-7.84 (m, 2H), 7.73-7.68 (m, 2H), 7.64 (t, J=8.0 Hz, 1H), 7.41 (s, 1H), 6.87 (d, J=7.6 Hz, 1H), 6.71 (d, J=8.0 Hz, 1H), 5.46 (s, 2H), 3.98 (s, 3H), 3.95 (s, 2H), 2.85 (br d, J=11.2 Hz, 2H), 2.63-2.57 (m, 1H), 2.16 (br s, 2H), 1.78-1.61 (m, 4H)

### Example A192—Preparation of Compound 148. 2-((4-(3-((4-Chloro-2-fluorobenzyl)oxy)phenyl)piperidin-1-yl)methyl)-4-(difluoromethoxy)-1-methyl-1H-benzo[d]imidazole-6-carboxylic acid (Compound 148) (3)

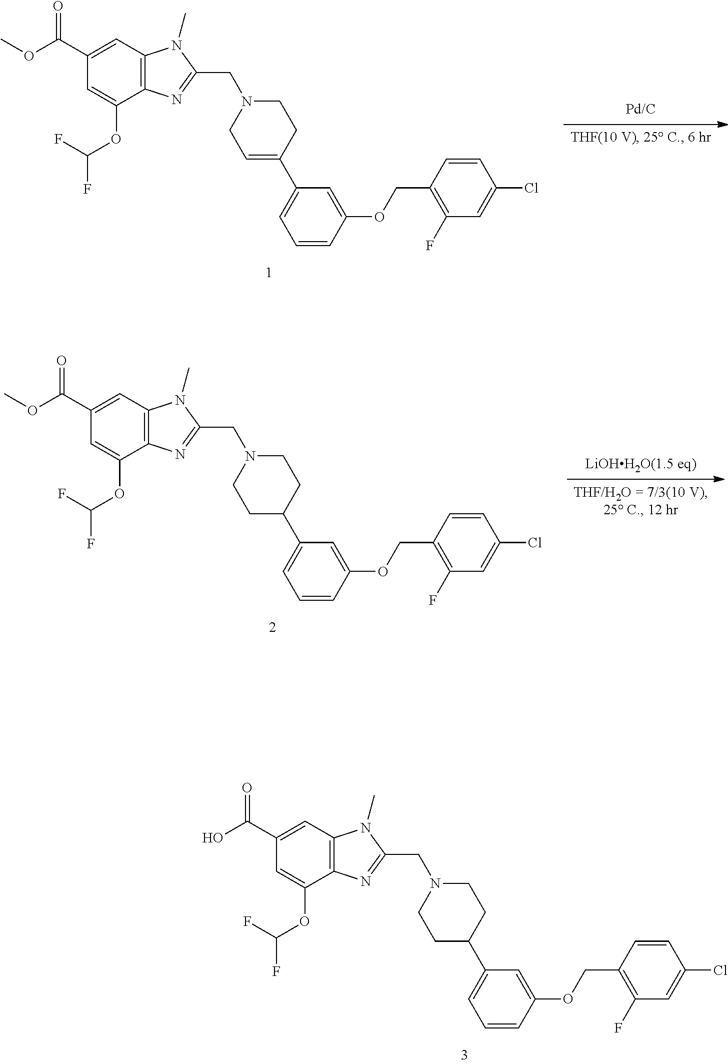

### General Procedure for Preparation of Intermediate 2:

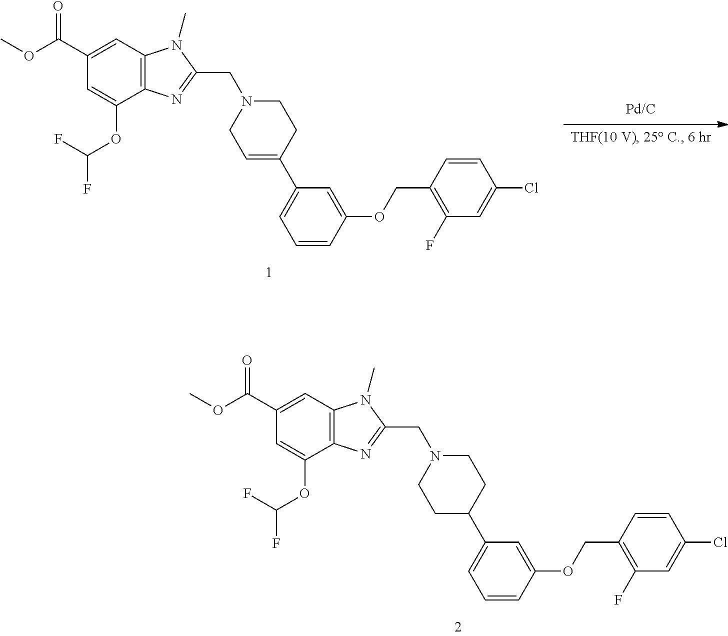 [SMILES: COC(=O)c1cc(OC(F)F)c2nc(CN3CC=C(c4cccc(OCc5ccc(Cl)cc5F)c4)CC3)n(C)c2c1 ; "Pd/C" ; "THF(10 V), 25° C., 6 hr" ; COC(=O)c1cc(OC(F)F)c2nc(CN3CCC(c4cccc(OCc5ccc(Cl)cc5F)c4)CC3)n(C)c2c1]

THF (1 mL) was charged to the Hydrogenating flask, then Pd/C (50 mg) was added to the mixture at 25° C. At 25° C., 1 (100 mg, 170.65 μmol, 1 eq) was added to the reaction mixture at 25° C. After the addition, the suspension was degassed under vacuum and purged with hydrogen several times. The mixture was stirred at 25° C. for 12 hr (15 psi). LCMS indicated 1 was consumed completely and one main peak with desired mass was detected. The mixture was filtered by diatomite. The filter cake was washed by THF (10 mL), the combined organic phase was filtered and concentrated in vacuum. 2 (80 mg, 136.05 μmol, 79.73% yield) was a yellow oil.

LCMS: RT=1.750 min, MS cal.: 587.2, [M+H]⁺=588.2

### General Procedure for Preparation of Compound 148 (3):

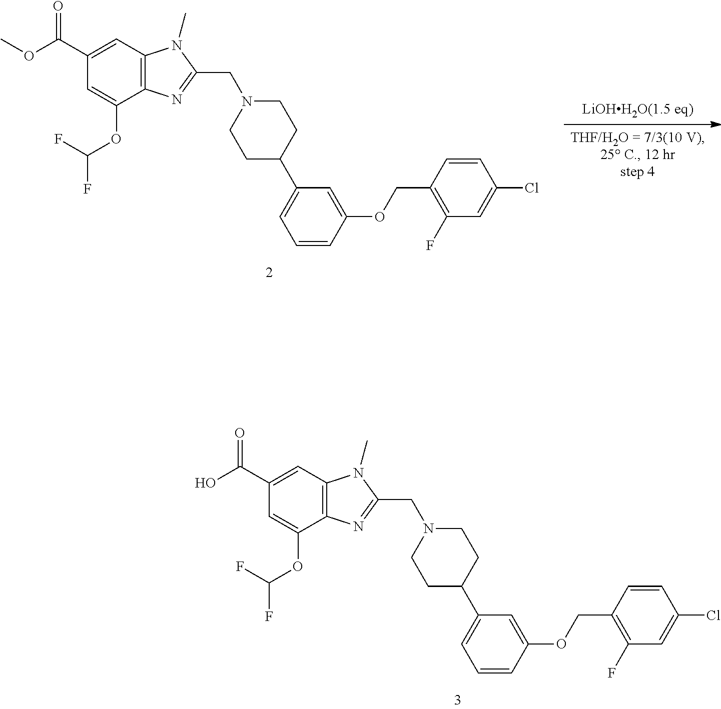

To a solution of 2 (70 mg, 119.04 μmol, 1 eq) in THF (0.56 mL) and H₂O (0.24 mL) was added LiOH·H₂O (7.49 mg, 178.57 μmol, 1.5 eq). The mixture was stirred at 25° C. for 12 hr. LCMS indicated 2 was consumed completely and one main peak with desired mass was detected. The reaction mixture was purified by prep-HPLC (column: Waters Xbridge Prep OBD C18 150*40 mm*10 um; mobile phase: [H₂O (10 mM NH₄HCO₃)-ACN]; gradient: 20%-70% B over 8.0 min). 2-((4-(3-((4-Chloro-2-fluorobenzyl)oxy)phenyl)piperidin-1-yl)methyl)-4-(difluoromethoxy)-1-methyl-1H-benzo[d]imidazole-6-carboxylic acid (27.8 mg, 46.50 μmol, 39.06% yield, 96% purity) was a white solid.

LCMS: RT=2.923 min, MS cal.: 539.2, [M+H]⁺=540.2

HPLC: RT=7.520 min, purity: 96.48%

¹H NMR (400 MHz, DMSO-d6) δ=8.05 (s, 1H), 7.62 (t, J=74.8 Hz, 1H), 7.58 (br t, J=8.0 Hz, 1H), 7.51 (s, 1H), 7.48 (dd, J=2.0, 10.0 Hz, 1H), 7.33 (dd, J=1.6, 8.3 Hz, 1H), 7.20 (t, J=8.0 Hz, 1H), 6.89 (s, 1H), 6.86-6.80 (m, 2H), 5.09 (s, 2H), 3.95 (s, 3H), 3.85 (s, 2H), 2.94 (br d, J=11.2 Hz, 2H), 2.48-2.44 (m, 1H), 2.26-2.16 (m, 2H), 1.78-1.69 (m, 2H), 1.69-1.57 (m, 2H)

### Example A193—Preparation of Compound 149. 2-((4-((6-((4-Cyano-2-fluorophenoxy)methyl)pyridin-2-yl)oxy)piperidin-1-yl)methyl)-4-(difluoromethoxy)-1-methyl-1H-benzo[d]imidazole-6-carboxylic acid (Compound 149) (7)

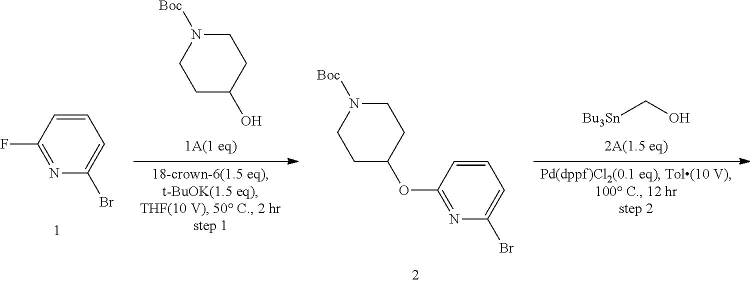

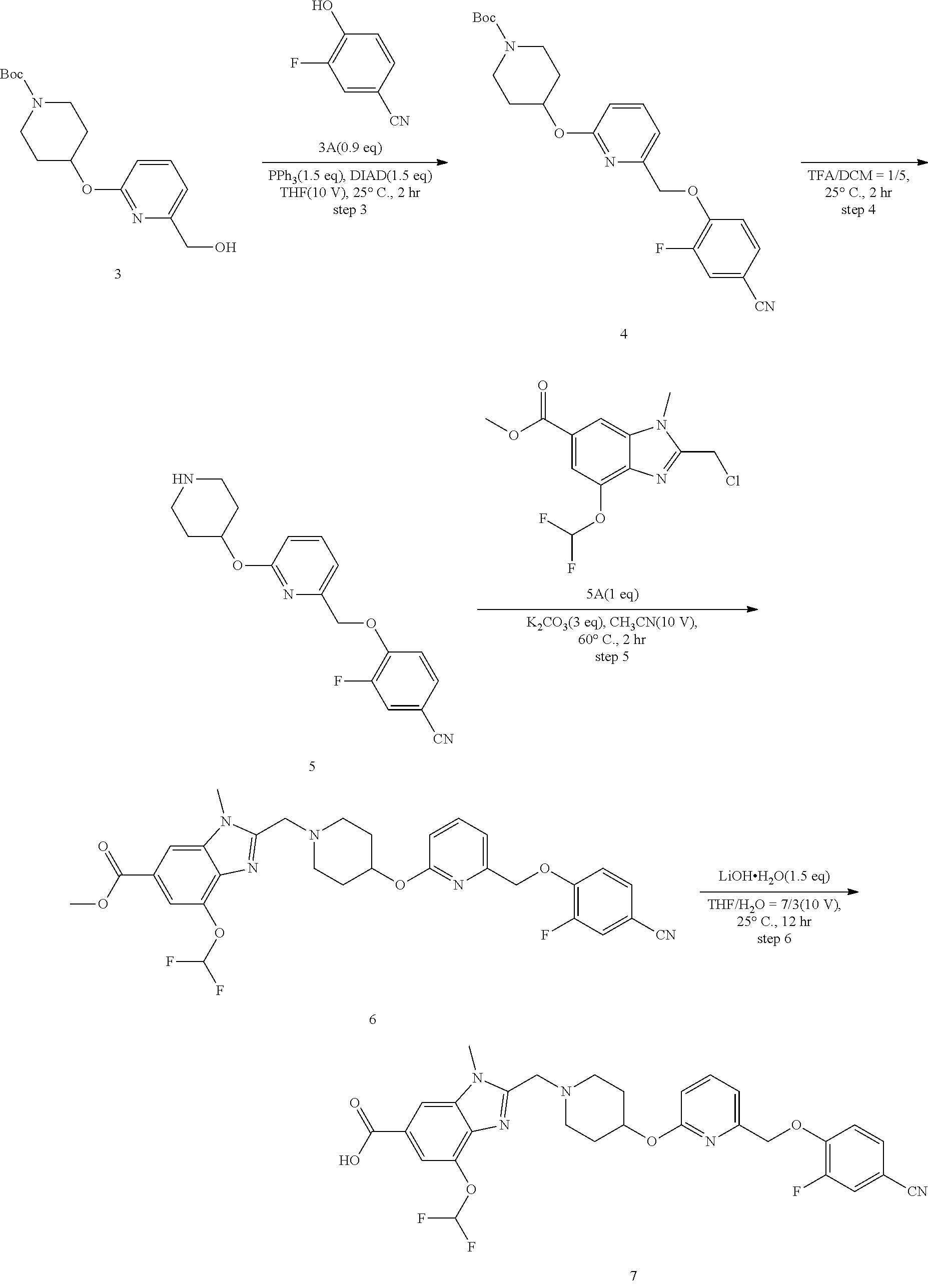

### General Procedure for Preparation of Intermediate 2:

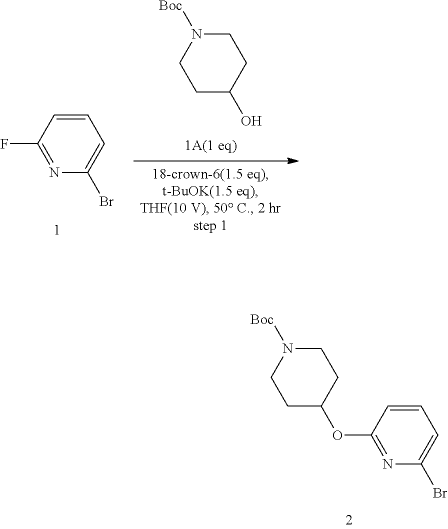

A mixture of 1 (5 g, 28.41 mmol, 1 eq), 1A (5.72 g, 28.41 mmol, 1 eq), 18-crown-6 (11.26 g, 42.62 mmol, 1.5 eq) and t-BuOK (4.78 g, 42.62 mmol, 1.5 eq) in THF (50 mL) was degassed and purged with N₂ for 3 times, and then the mixture was stirred at 50° C. for 2 hr under N₂ atmosphere. TLC indicated 1 was consumed completely and one new spot formed. The reaction was clean according to TLC (Petroleum ether/Ethyl acetate=10/1, Product Rf=0.46). The residue mixture was diluted with H₂O 50 mL and extracted with EtOAc 50 mL*3. The reaction mixture was poured into separatory funnel and separated. The combined organic layers were washed with brine 50 mL, dried over Na₂SO₄, filtered, and concentrated under reduced pressure to give a residue. The residue was purified by column chromatography (SiO₂, Petroleum ether/Ethyl acetate=100/1 to 95/5, TLC-Petroleum ether/Ethyl acetate=10/1, Produce R_{f}=0.46). 2 (7.9 g, 21.43 mmol, 75.45% yield, 96.93% purity) was obtained as a white solid.

LCMS: RT=1.502 min, MS cal.: 356.07/358.07, [M−H]⁺=356.9/358.9

¹H NMR (400 MHz, CHCl3-d) δ=7.41 (t, J=8.0 Hz, 1H), 7.04 (d, J=7.6 Hz, 1H), 6.66 (d, J=8.0 Hz, 1H), 5.22 (tt, J=3.6, 7.8 Hz, 1H), 3.75 (ddd, J=4.0, 6.8, 13.2 Hz, 2H), 3.36-3.26 (m, 2H), 2.02-1.92 (m, 2H), 1.72 (dtd, J=4.0, 8.4, 12.8 Hz, 2H), 1.48 (s, 9H)

### General Procedure for Preparation of Intermediate 3:

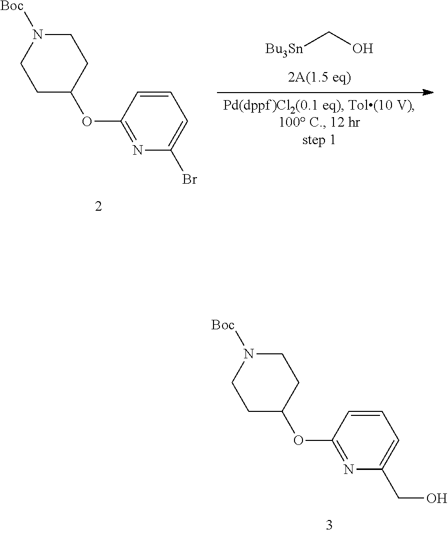

To a solution of 2 (6 g, 16.80 mmol, 1 eq) in Toluene (60 mL) was added 2A (8.09 g, 25.19 mmol, 1.5 eq). The mixture was degassed and purged with N₂ for 3 times and then added Pd (dppf) C12 (1.23 g, 1.68 mmol, 0.1 eq) at 20° C. The mixture was stirred at 100° C. for 12 hr under N₂ atmosphere. LCMS (product: RT=1.224 min) showed the 2 was consumed completely, one main peak with desired mass was detected. The reaction mixture was cooled down to 20° C. then filtered through a pad of Celite, the filter cake was washed with EtOAc 10 mL*2. The filtrate was added H₂O 100 mL and extracted with EtOAc 100 mL*3. The combined organic layers were washed with brine 50 mL*2, dried over Na₂SO₄, filtered, and concentrated under reduced pressure to give a residue. The residue was purified by column chromatography (SiO₂, Petroleum ether/Ethyl acetate=10/1 to 1/1). 3 (1.6 g, 5.19 mmol, 30.89% yield) was obtained as a colorless oil.

¹H NMR (400 MHz, CHCl3-d) δ=7.57 (t, J=7.6 Hz, 1H), 6.80 (d, J=7.2 Hz, 1H), 6.62 (d, J=8.4 Hz, 1H), 5.24 (tt, J=4.0, 7.6 Hz, 1H), 4.65 (d, J=5.0 Hz, 2H), 3.83-3.65 (m, 2H), 3.41-3.24 (m, 3H), 2.07-1.88 (m, 2H), 1.75 (dtd, J=4.0, 8.0, 12.4 Hz, 2H), 1.47 (s, 9H)

### General Procedure for Preparation of Intermediate 4:

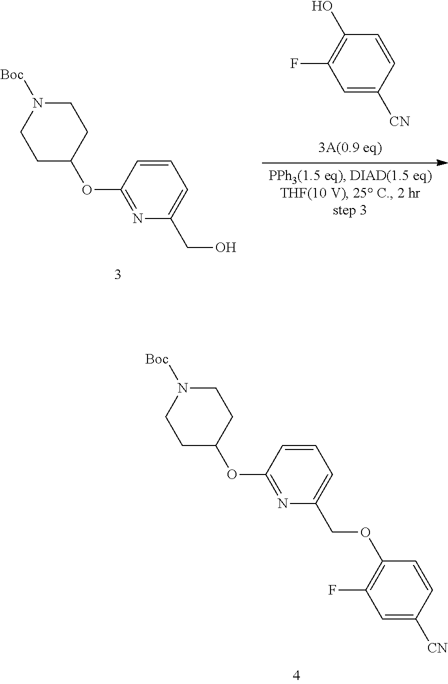 [SMILES: CC(C)(C)OC(=O)N1CCC(Oc2cccc(CO)n2)CC1 ; N#Cc1ccc(O)c(F)c1 ; "3A(0.9 eq)" ; "PPh3(1.5 eq), DIAD(1.5 eq)THF(10 V), 25° C., 2 hrstep 3" ; CC(C)(C)OC(=O)N1CCC(Oc2cccc(COc3ccc(C#N)cc3F)n2)CC1]

A mixture of 3 (190 mg, 616.14 μmol, 1 eq), 3A (76.03 mg, 554.53 μmol, 0.9 eq), PPh₃ (242.41 mg, 924.21 μmol, 1.5 eq), DIAD (186.88 mg, 924.21 μmol, 179.18 μL, 1.5 eq) in THF (1.9 mL) was degassed and purged with N₂ for 3 times, and then the mixture was stirred at 25° C. for 2 hr under N₂ atmosphere. LCMS (product: RT=1.513 min) showed the 3 was consumed completely. The reaction mixture was quenched by addition H₂O 3 mL at 20° C. and extracted with ethyl acetate 3 mL*3. The combined organic layers were washed with brine 3 mL*2, dried over Na₂SO₄, filtered, and concentrated under reduced pressure to give a residue. The residue was purified by column chromatography (SiO₂, Petroleum ether/Ethyl acetate=10/1 to 1/1). 4 (180 mg, 421.08 μmol, 68.34% yield) was obtained as a white solid.

¹H NMR (400 MHz, CHCl3-d) δ=7.61 (t, J=8.0 Hz, 1H), 7.43-7.37 (m, 2H), 7.13-7.06 (m, 1H), 7.03 (d, J=7.2 Hz, 1H), 6.67 (d, J=8.4 Hz, 1H), 5.23-5.14 (m, 3H), 3.82-3.67 (m, 2H), 3.36-3.23 (m, 2H), 2.00-1.88 (m, 2H), 1.78-1.62 (m, 3H), 1.48 (s, 9H)

### General Procedure for Preparation of Intermediate 5:

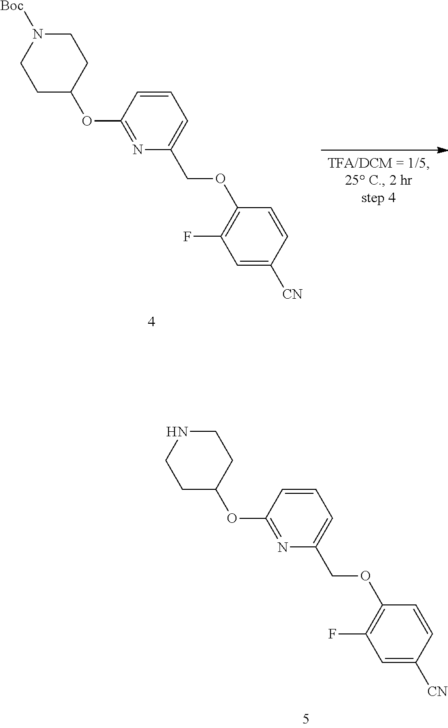

A mixture of 4 (180 mg, 421.08 μmol, 1 eq), in TFA (0.36 mL) and DCM (1.8 mL) was degassed and purged with N₂ for 3 times, and then the mixture was stirred at 25° C. for 2 hr under N₂ atmosphere. LCMS (product: RT=0.356 min) showed the 4 was consumed completely. The reaction mixture was concentrated under reduced pressure to remove solvent. The residue was diluted with ethyl acetate 2 mL and adjusted to pH=8-9 with sat. Na₂CO₃, extracted with ethyl acetate 3 mL*3. The combined organic layers were washed with brine 3 mL*2, dried over Na₂SO₄, filtered, and concentrated under reduced pressure to give a residue. 5 (180 mg, crude) was obtained as a white solid.

### General Procedure for Preparation of Intermediate 6:

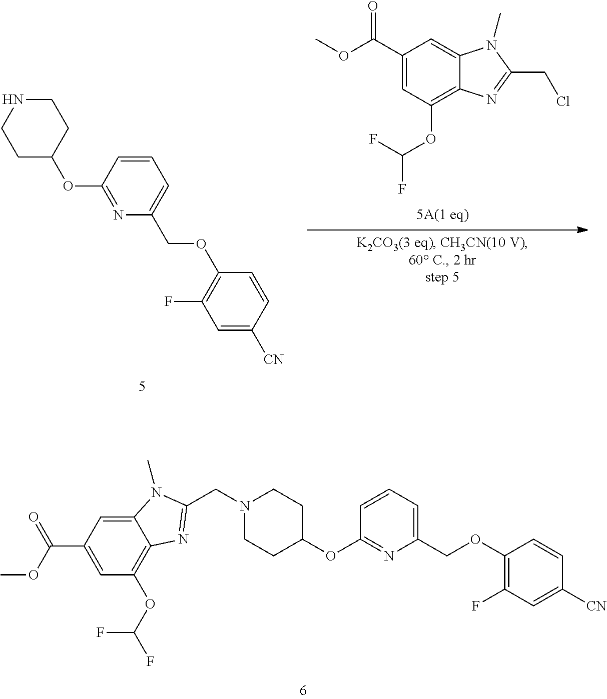 [SMILES: N#Cc1ccc(OCc2cccc(OC3CCNCC3)n2)c(F)c1 ; COC(=O)c1cc(OC(F)F)c2nc(CCl)n(C)c2c1 ; "5A(1 eq)" ; "K2CO3(3 eq), CH3CN(10 V),60° C., 2 hrstep 5" ; COC(=O)c1cc(OC(F)F)c2nc(CN3CCC(Oc4cccc(COc5ccc(C#N)cc5F)n4)CC3)n(C)c2c1]

To a solution of 5 (180 mg, 549.87 μmol, 1 eq) and 5A (167.53 mg, 549.87 μmol, 1 eq) in CH₃CN (1.8 mL) was added K₂CO₃ (227.98 mg, 1.65 mmol, 3 eq). The mixture was stirred at 60° C. for 2 hr. LCMS (product: RT=0.443 min) showed the 5 was consumed completely. The reaction mixture was quenched by addition H₂O 3 mL at 20° C. and extracted with ethyl acetate 3 mL*3. The combined organic layers were washed with brine 3 mL*2, dried over Na₂SO₄, filtered, and concentrated under reduced pressure to give a residue. The residue was purified by column chromatography (SiO₂, Petroleum ether/Ethyl acetate=1/1 to 0/1). 6 (170 mg, 285.44 μmol, 51.91% yield) was obtained as a brown solid.

¹H NMR (400 MHz, CHCl3-d) δ=7.99 (s, 1H), 7.71 (s, 1H), 7.60 (t, J=7.6 Hz, 1H), 7.43-7.36 (m, 2H), 7.31 (t, J=74 Hz, 1H), 7.14-7.09 (m, 1H), 7.02 (d, J=7.2 Hz, 1H), 6.66 (d, J=8.0 Hz, 1H), 5.19 (s, 2H), 5.07 (s, 1H), 3.98 (s, 3H), 3.97 (s, 3H), 3.90 (s, 2H), 2.82 (s, 2H), 2.45 (s, 2H), 2.09-1.94 (m, 2H), 1.81 (s, 2H)

### General Procedure for Preparation of Compound 149 (7):

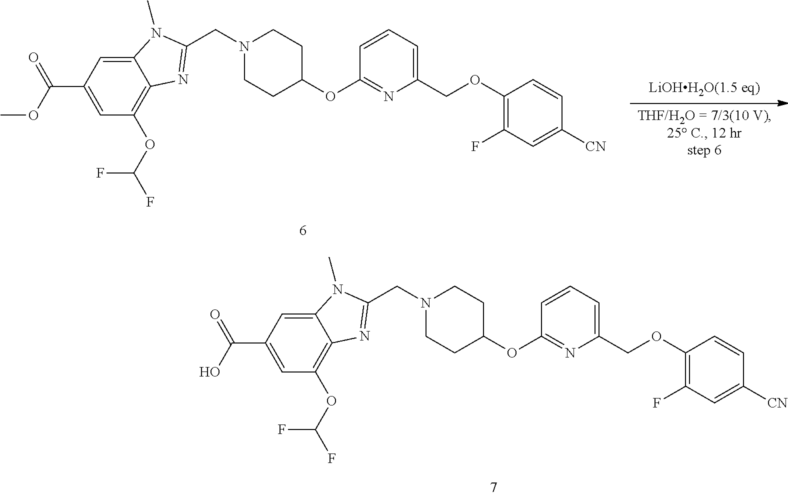

To a solution of 6 (150 mg, 251.86 μmol, 1 eq), in THF (1.05 mL) was added LiOH·H₂O (15.85 mg, 377.79 μmol, 1.5 eq), in H₂O (0.45 mL). The mixture was stirred at 25° C. for 12 hr under N₂ atmosphere. LCMS (product: RT=0.402 min) showed the 6 was consumed completely. The reaction mixture was concentrated under reduced pressure to remove solvent to give a residue. The residue was purified by prep-HPLC (column: Waters Xbridge Prep OBD C18 150*40 mm*10 um; mobile phase: [H₂O (10 mM NH₄HCO₃)—CH₃CN]; gradient: 15%-65% B over 8.0 min). 2-((4-((6-((4-Cyano-2-fluorophenoxy)methyl)pyridin-2-yl)oxy)piperidin-1-yl)methyl)-4-(difluoromethoxy)-1-methyl-1H-benzo[d]imidazole-6-carboxylic acid (27.62 mg, 46.92 μmol, 18.63% yield, 98.80% purity) was obtained as a white solid.

LCMS: RT=2.583 min, MS cal.: 581.2, [M+H]⁺=582.2

HPLC: RT=10.587 min

¹H NMR (400 MHz, DMSO-d6) δ=8.05 (s, 1H), 7.87 (dd, J=1.6, 11.2 Hz, 1H), 7.72 (t, J=8.0 Hz, 1H), 7.66 (d, J=8.8 Hz, 1H), 7.63 (t, J=74 Hz, 1H), 7.51 (s, 1H), 7.47-7.41 (m, 1H), 7.05 (d, J=7.2 Hz, 1H), 6.73 (d, J=8.4 Hz, 1H), 5.30 (s, 2H), 4.90 (td, J=4.4, 8.4 Hz, 1H), 3.94 (s, 3H), 3.84 (s, 2H), 2.79-2.70 (m, 2H), 2.30 (t, J=9.6 Hz, 2H), 1.90 (d, J=9.6 Hz, 2H), 1.61 (q, J=8.8 Hz, 2H)

### Example A194—Preparation of Compound 150. 2-((4-(6-((4-Chloro-2-fluorobenzyl)oxy)pyridin-2-yl) piperazin-1-yl)methyl)-4-(difluoromethoxy)-1-methyl-1H-benzo[d]imidazole-6-carboxylic acid (Compound 150) (5)

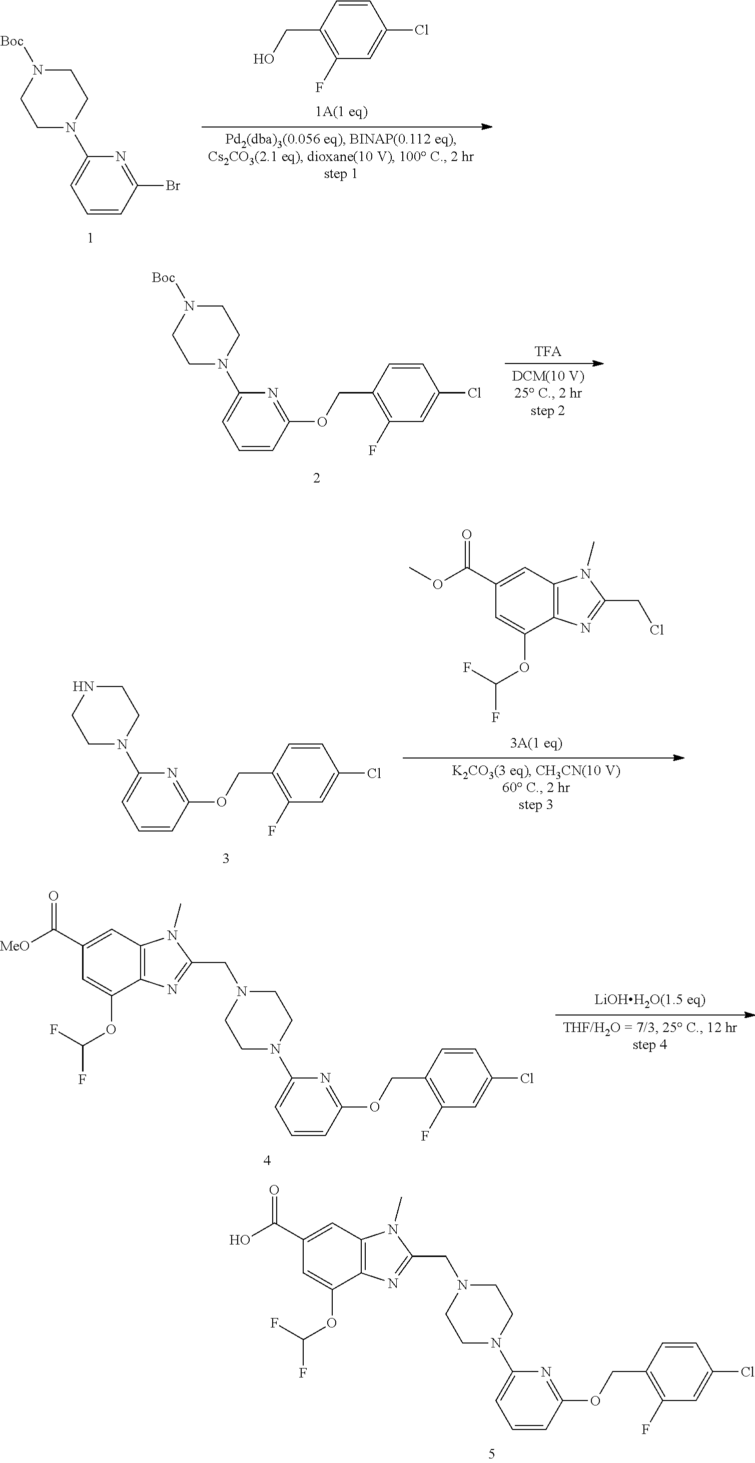

### General Procedure for Preparation of Intermediate 2:

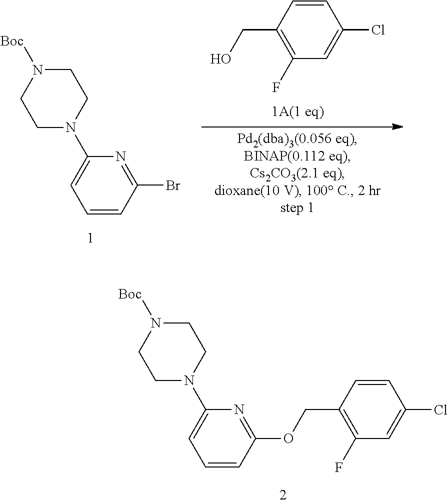

To a solution of 1 (400 mg, 1.17 mmol, 1 eq) in dioxane (4 mL) was added 1A (187.68 mg, 1.17 mmol, 1 eq), BINAP (81.51 mg, 130.91 μmol, 0.112 eq), Cs₂CO₃ (799.72 mg, 2.45 mmol, 2.1 eq) and Pd₂(dba)₃ (59.94 mg, 65.45 μmol, 0.056 eq). The mixture was stirred at 100° C. for 2 hr. LCMS showed 1 was consumed completely and one main peak with desired mass was detected. The reaction mixture was diluted with H₂O 10 mL and extracted with EtOAc 60 mL (20 mL*3). The combined organic layers were washed with brine 20 mL (10 mL*2), dried over Na₂SO₄, filtered and concentrated under reduced pressure to give a residue. The residue was purified by column chromatography (SiO₂, PE/EtOAc=1/0 to 4/1). 2 (100 mg, 229.92 μmol, 19.67% yield) was obtained as a yellow oil.

LCMS: RT=1.703 min, MS cal.: 421.2/423.2, [M+H]⁺=422.0/424.0

¹HNMR (400 MHz, CHCl3-d) δ=7.43 (q, J=8.4 Hz, 2H), 7.15-7.09 (m, 2H), 6.20 (dd, J=8.0, 16.0 Hz, 2H), 5.36 (s, 2H), 3.56-3.47 (m, 8H), 1.50 (s, 9H)

### General Procedure for Preparation of Intermediate 3:

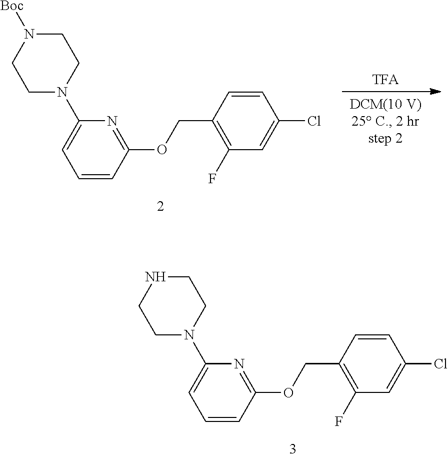 [SMILES: CC(C)(C)OC(=O)N1CCN(c2cccc(OCc3ccc(Cl)cc3F)n2)CC1 ; "DCM(10 V)25° C., 2 hrstep 2" ; Fc1cc(Cl)ccc1COc1cccc(N2CCNCC2)n1 ; "TFA"]

To a solution of Compound 2 (90 mg, 213.32 μmol, 1 eq) in DCM (1 mL) was added TFA (460.50 mg, 4.04 mmol, 0.3 mL, 18.93 eq). The mixture was stirred at 25° C. for 2 hr. LCMS showed 2 was consumed completely and one main peak with desired mass was detected. The reaction mixture was concentrated under reduced pressure. 3 (90 mg, 206.52 μmol, 96.81% yield, TFA salt) was obtained as a colorless oil.

LCMS: RT=1.200 min, MS cal.: 321.1/323.1, [M+H]⁺=322.0/324.0

¹HNMR (400 MHz, CHCl3-d) δ=7.58 (br t, J=8.0 Hz, 1H), 7.41 (t, J=8.0 Hz, 1H), 7.18-7.11 (m, 2H), 6.35-6.28 (m, 2H), 5.34 (s, 2H), 3.89-3.83 (m, 4H), 3.41 (br s, 4H)

### General Procedure for Preparation of Intermediate 4:

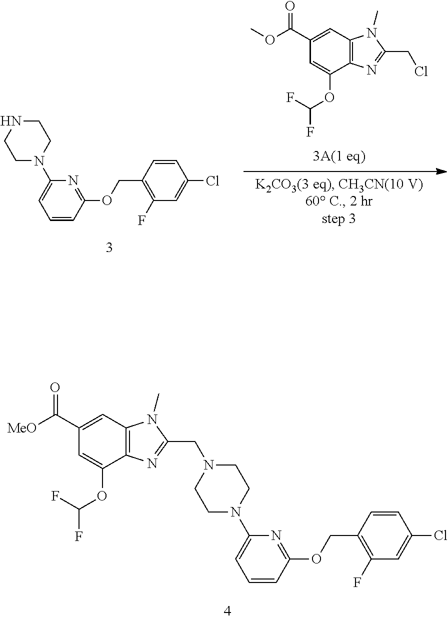 [SMILES: Fc1cc(Cl)ccc1COc1cccc(N2CCNCC2)n1 ; COC(=O)c1cc(OC(F)F)c2nc(CCl)n(C)c2c1 ; "3A(1 eq)" ; "K2CO3(3 eq), CH3CN(10 V)60° C., 2 hrstep 3" ; COC(=O)c1cc(OC(F)F)c2nc(CN3CCN(c4cccc(OCc5ccc(Cl)cc5F)n4)CC3)n(C)c2c1]

To a solution of 3 (90 mg, 206.52 μmol, 1 eq, TFA salt) in CH₃CN (1 mL) was added K₂CO₃ (85.63 mg, 619.55 μmol, 3 eq) and 3A (62.92 mg, 206.52 μmol, 1 eq). The mixture was stirred at 60° C. for 2 hr. LCMS showed 6% of 3 remained. Several new peaks were shown on LCMS and 56% of desired 4 was detected. The reaction mixture was diluted with H₂O 5 mL and extracted with EtOAc 20 mL (10 mL*2). The combined organic layers were washed with brine mL, dried over Na₂SO₄, filtered, and concentrated under reduced pressure to give a residue. The residue was purified by column chromatography (SiO₂, PE/EtOAc=1/0 to 2/1). 4 (60 mg, 97.63 μmol, 47.27% yield) was obtained as a brown solid.

LCMS: RT=1.650 min, MS cal.: 589.2/591.2, [M+H]⁺=590.0/592.0

### General Procedure for Preparation of Compound 150 (5):

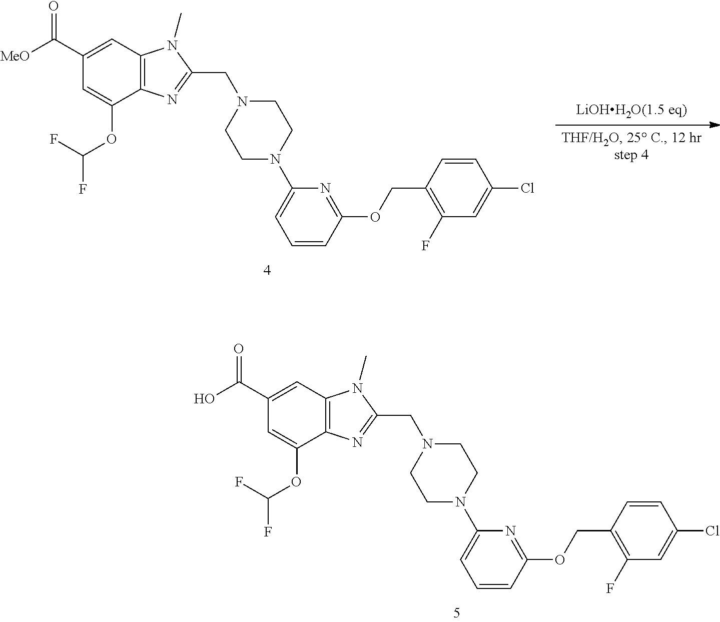 [SMILES: COC(=O)c1cc(OC(F)F)c2nc(CN3CCN(c4cccc(OCc5ccc(Cl)cc5F)n4)CC3)n(C)c2c1 ; "LiOH·H2O(1.5 eq)" ; "THF/H2O, 25° C., 12 hrstep 4" ; Cn1c(CN2CCN(c3cccc(OCc4ccc(Cl)cc4F)n3)CC2)nc2c(OC(F)F)cc(C(=O)O)cc21]

To a solution of 4 (40 mg, 67.80 μmol, 1 eq) in THF (0.7 mL) and H₂O (0.3 mL) was added LiOH·H₂O (4.27 mg, 101.70 μmol, 1.5 eq). The mixture was stirred at 25° C. for 12 hr. LCMS showed 19% of 4 remained. Several new peaks were shown on LCMS and 76% of desired product was detected. The mixture was diluted with CH₃CN 1 mL. The residue was purified by prep-HPLC (neutral condition: column: Waters Xbridge Prep OBD C18 150*40 mm*10 um; mobile phase: [H₂O (10 mM NH₄HCO₃)-ACN]; gradient: 20%-80% B over 8.0 min). 2-((4-(6-((4-Chloro-2-fluorobenzyl)oxy)pyridin-2-yl) piperazin-1-yl)methyl)-4-(difluoromethoxy)-1-methyl-1H-benzo[d]imidazole-6-carboxylic acid (18.6 mg, 31.32 μmol, 46.20% yield) was obtained as a brown solid.

LCMS: RT=2.793 min, MS cal.: 575.2/577.2, [M+H]⁺=576.2/578.2

HPLC: RT=12.424 min

¹H NMR (400 MHz, DMSO-d₆) δ=8.10 (s, 1H), 7.71-7.67 (m, 1H), 7.46-7.39 (m, 2H), 7.24 (t, J=74 Hz 1H), 7.22-7.15 (m, 2H), 6.27 (d, J=8.0 Hz, 1H), 6.10 (d, J=8.0 Hz, 1H), 5.33 (s, 2H), 4.02 (s, 3H), 3.92 (s, 2H), 3.54-3.46 (m, 5H), 2.62 (t, J=4.8 Hz, 4H)

### Example A195—Preparation of Compound 151. 2-((4-(6-(6-Cyano-8-fluoro-3,4-dihydroisoquinolin-2(1H)-yl)pyridin-2-yl)piperidin-1-yl)methyl)-4-(difluoromethoxy)-1-methyl-1H-benzo[d]imidazole-6-carboxylic acid (Compound 151) (7)

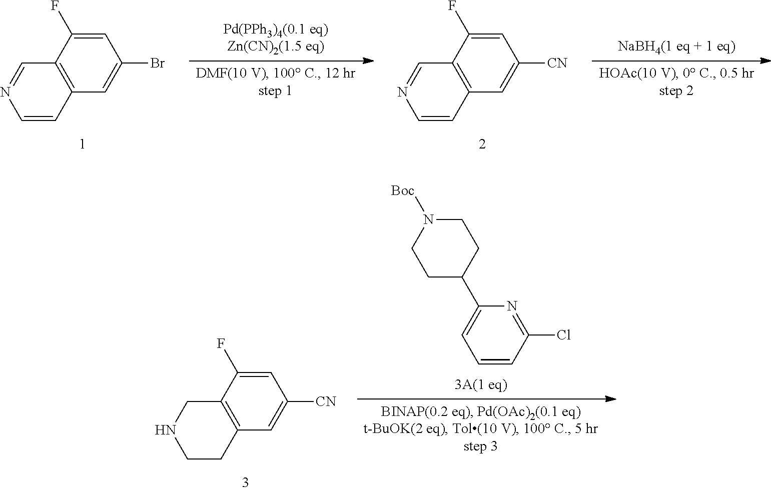

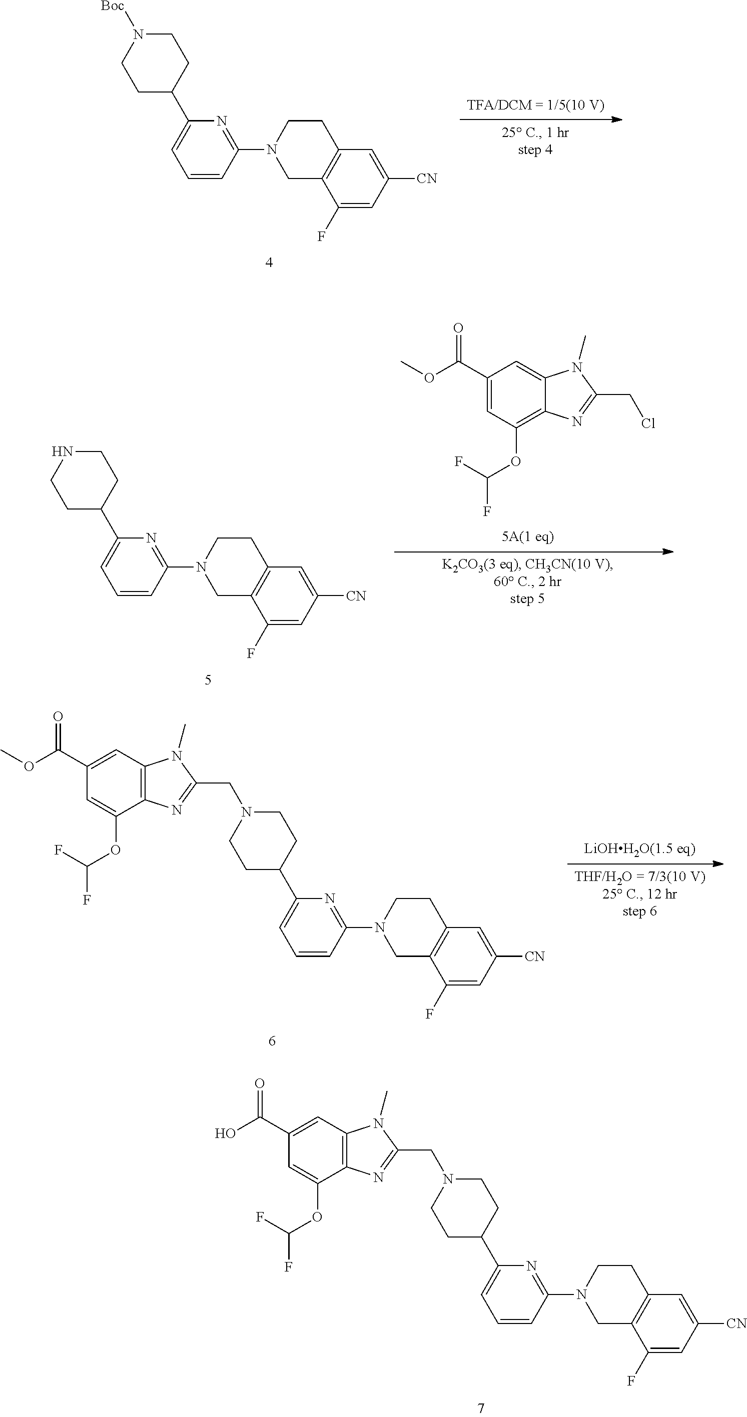

### General Procedure for Preparation of Intermediate 2:

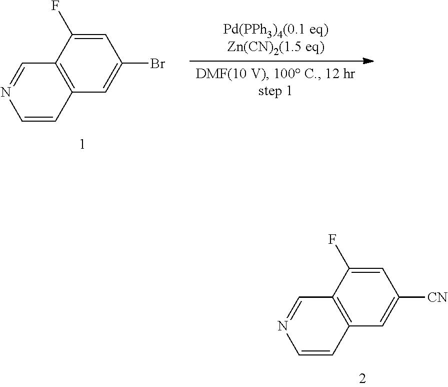 [SMILES: Fc1cc(Br)cc2ccncc12 ; "DMF(10 V), 100° C., 12 hrstep 1" ; "Pd(PPh3)4(0.1 eq)Zn(CN)2(1.5 eq)" ; N#Cc1cc(F)c2cnccc2c1]

DMF (5 mL) was charged to the round bottom flask, then 1 (500 mg, 2.21 mmol, 1 eq) was added to the mixture at 25° C. At 25° C., Zn(CN)₂ (389.61 mg, 3.32 mmol, 210.60 μL, 1.5 eq) and Pd(PPh₃)₄ (255.60 mg, 221.19 μmol, 0.1 eq) was added dropwise to the reaction mixture at 25° C. After the addition, the mixture was stirred at 100° C. for 12 hr. LCMS showed 1 was consumed completely and one main peak with desired mass was detected. The reaction mixture was added to H₂O (10 mL) at 25° C. The mixture was extracted by EtOAc (10 mL). Then organic phase was combined and washed by H₂O (5 mL), brine (5 mL), dried with Na₂SO₄, filtered. The organic phase was concentrated under reduced pressure at 35° C. to give a residue. The residue was purified by column chromatography (SiO₂, Petroleum ether/Ethyl acetate=10/1 to 0/1). 2 (310 mg, 1.80 mmol, 81.41% yield) was obtained as a yellow solid.

LCMS: RT=1.332 min, MS cal.: 172.2, [M+H]⁺=173.2

¹H NMR (400 MHz, CHCl3-d) δ=9.64 (s, 1H), 8.79 (d, J=5.6 Hz, 1H), 8.06 (s, 1H), 7.77 (d, J=5.6 Hz, 1H), 7.44 (d, J=9.2 Hz, 1H)

General procedure for preparation of Intermediate 3:

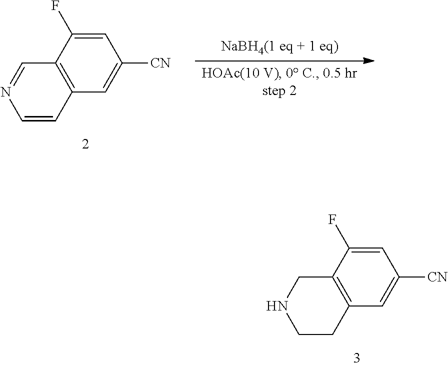 [SMILES: N#Cc1cc(F)c2cnccc2c1 ; "HOAc(10 V), 0° C., 0.5 hrstep 2" ; "NaBH4(1 eq + 1 eq)" ; N#Cc1cc(F)c2c(c1)CCNC2]

AcOH (3 mL) was charged to the three-necked round bottom flask, then 2 (300 mg, 1.74 mmol, 1 eq) and was added to the mixture at 25° C. At 10° C., NaBH₄ (65.93 mg, 1.74 mmol, 1 eq) was added in portions to the reaction mixture at 10° C. After the addition, the mixture was stirred at 10° C. for 10 min. At 10° C., NaBH₄ (65.93 mg, 1.74 mmol, 1 eq) was added in portions to the reaction mixture at 10° C. After the addition, the mixture was stirred at 10° C. for 10 min. TLC (PE:EA=1:1, Rf=0.21) indicated 2 was consumed completely and one new spot formed. The reaction was clean according to TLC. The reaction mixture was added with aq. NaHCO₃ to adjust pH=7. The mixture was extracted by DCM (15 mL). Then organic phase was combined and washed by H₂O (10 mL), brine (10 mL), dried with Na₂SO₄, filtered. The organic phase was concentrated under reduced pressure at 25° C. to give a residue. 3 (160 mg, 908.11 μmol, 52.11% yield) was obtained as a yellow solid.

¹H NMR (400 MHz, MeOH-d4) δ=7.40 (s, 1H), 7.35 (d, J=9.2 Hz, 1H), 4.09 (s, 2H), 3.17 (t, J=6.0 Hz, 2H), 2.96-2.90 (m, 2H)

### General Procedure for Preparation of Intermediate 4:

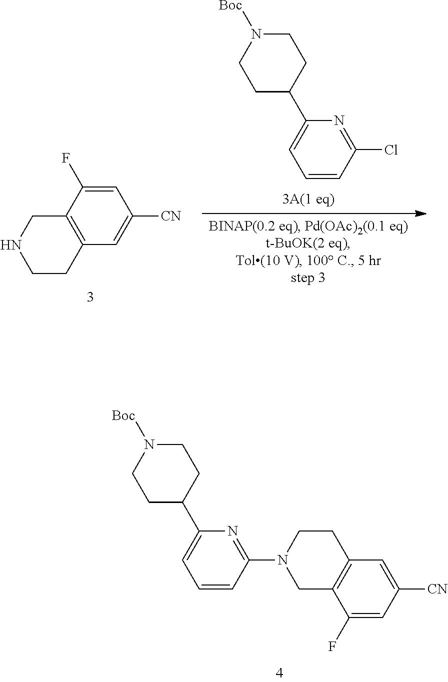

A mixture of 3 (100 mg, 567.57 μmol, 1 eq), 3A (168.45 mg, 567.57 μmol, 1 eq), Pd(OAc)₂ (12.74 mg, 56.76 μmol, 0.1 eq), t-BuOK (127.38 mg, 1.14 mmol, 567.57 μL, 2 eq), BINAP (70.68 mg, 113.51 μmol, 0.2 eq) in Toluene (1 mL) was degassed and purged with N₂ for 3 times, and then the mixture was stirred at 100° C. for 5 hr under N₂ atmosphere. LCMS showed 3 was consumed completely and one main peak with desired mass was detected. The combined mixture was diluted with H₂O (5 mL) and extracted with EtOAc (5 mL*3). The combined organic layers were washed with brine (3 mL*3), dried over Na₂SO₄, filtered and concentrated under reduced pressure to give a residue. The residue was purified by column chromatography (SiO₂, Petroleum ether/Ethyl acetate=10/1 to 0/1). 4 (100 mg, 160.36 μmol, 28.25% yield, 70% purity) was obtained as yellow oil.

LCMS: RT=0.508 min, MS cal.: 436.2, [M+H]⁺=437.2

### General Procedure for Preparation of Intermediate 5:

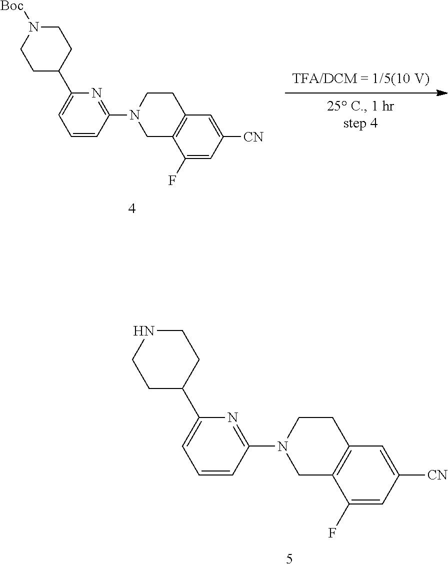

A mixture of 4 (90 mg, 206.18 μmol, 1 eq) in DCM (1 mL) and TFA (0.2 mL) was degassed and purged with N₂ for 3 times, and then the mixture was stirred at 25° C. for 1 hr under N₂ atmosphere. LCMS showed 4 was consumed completely and one main peak with desired mass was detected. The reaction mixture was concentrated under reduced pressure to give a residue. 5 (90 mg, 199.81 μmol, 96.91% yield, TFA) was obtained as yellow oil.

LCMS: RT=0.318 min, MS cal.: 336.2, [M+H]⁺=337.1

### General Procedure for Preparation of Intermediate 6:

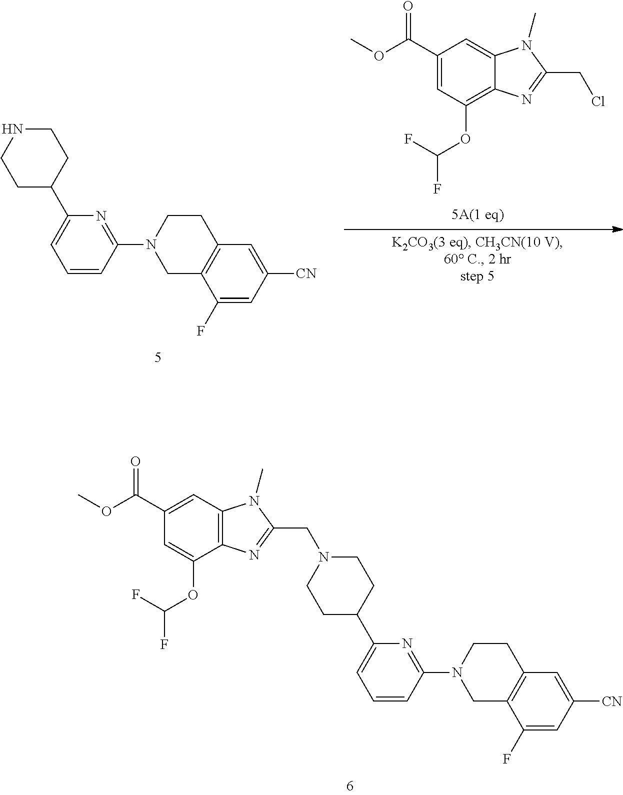 [SMILES: N#Cc1cc(F)c2c(c1)CCN(c1cccc(C3CCNCC3)n1)C2 ; COC(=O)c1cc(OC(F)F)c2nc(CCl)n(C)c2c1 ; "5A(1 eq)" ; "K2CO3(3 eq), CH3CN(10 V),60° C., 2 hrstep 5" ; COC(=O)c1cc(OC(F)F)c2nc(CN3CCC(c4cccc(N5CCc6cc(C#N)cc(F)c6C5)n4)CC3)n(C)c2c1]

A mixture of 5 (90 mg, 199.81 μmol, 1 eq, TFA), 5A (60.88 mg, 199.81 μmol, 1 eq), K₂CO₃ (82.84 mg, 599.43 μmol, 3 eq) in CH₃CN (1 mL) was degassed and purged with N₂ for 3 times, and then the mixture was stirred at 60° C. for 2 hr under N₂ atmosphere. LCMS showed 5 was consumed completely and one main peak with desired mass was detected. The combined mixture was diluted with H₂O (5 mL) and extracted with EtOAc (10 mL*3). The combined organic layers were washed with brine (10 mL*3), dried over Na₂SO₄, filtered and concentrated under reduced pressure to give a residue. The residue was purified by column chromatography (SiO₂, Petroleum ether/Ethyl acetate=10/1 to 0/1). 6 (70 mg, 115.78 μmol, 57.94% yield) was obtained as a yellow solid.

LCMS: RT=0.441 min, MS cal.: 604.2, [M+H]⁺=605.3

### General Procedure for Preparation of Compound 151 (7):

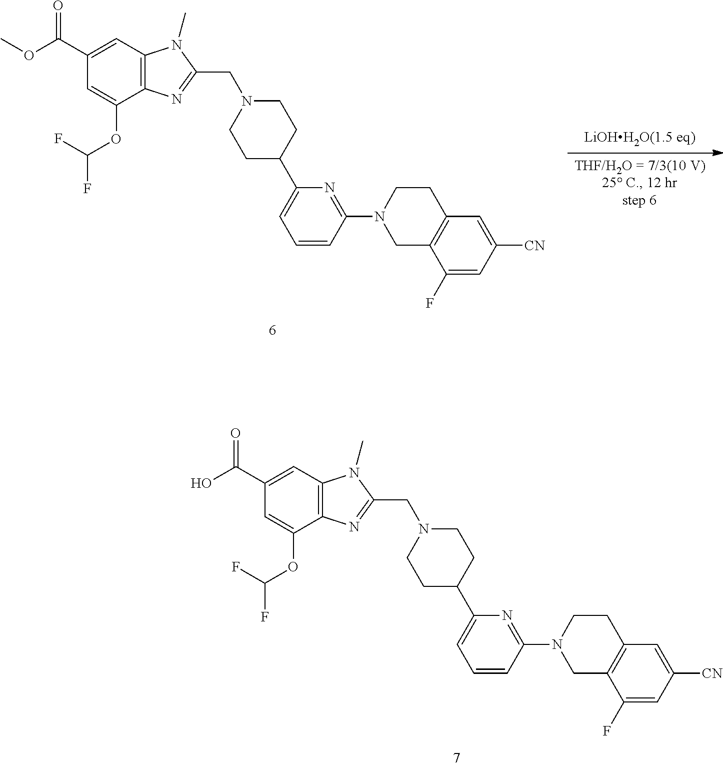

A mixture of Compound 6 (60 mg, 99.24 μmol, 1 eq), LiOH·H₂O (6.25 mg, 148.85 μmol, 1.5 eq) in THF (0.7 mL) and H₂O (0.3 mL) was degassed and purged with N₂ for 3 times, and then the mixture was stirred at 25° C. for 12 hr under N₂ atmosphere. LCMS showed Compound 6 was consumed completely and one main peak with desired mass was detected. The combined mixture was added with 0.5M citric acid to adjust pH=7. The mixture was purified by prep-HPLC (column: Waters Xbridge Prep OBD C18 150*40 mm*10 um; mobile phase: [H₂O (10 mM NH₄HCO₃)-ACN]; gradient: 25%-65% B over 8.0 min). 2-((4-(6-(6-Cyano-8-fluoro-3,4-dihydroisoquinolin-2(1H)-yl)pyridin-2-yl)piperidin-1-yl)methyl)-4-(difluoromethoxy)-1-methyl-1H-benzo[d]imidazole-6-carboxylic acid (10.14 mg, 16.59 μmol, 16.71% yield, 96.61% purity) was obtained as a white solid.

LCMS: RT=2.746 min, MS cal.: 590.6, [M+H]⁺=591.3

HPLC: RT=9.811 min, purity: 96.61%

¹H NMR (400 MHz, DMSO-d6) δ=8.05 (s, 1H), 7.69-7.65 (m, 1H), 7.62 (t, J=74.4 Hz, 1H), 7.57 (s, 1H), 7.52-7.47 (m, 2H), 6.79-6.74 (m, 1H), 6.58-6.54 (m, 1H), 4.76 (s, 2H), 3.95 (s, 3H), 3.86-3.79 (m, 4H), 2.92 (br s, 4H), 2.56-2.54 (m, 1H), 2.26-2.17 (m, 2H), 1.83-1.64 (m, 4H)

### Example A196—Preparation of Compound 152. 2-((4-(3-((4-Chloro-2-fluorobenzyl)oxy)-4-fluorophenyl)-3,6-dihydropyridin-1(2H)-yl)methyl)-4-(difluoromethoxy)-1-methyl-1H-benzo[d]imidazole-6-carboxylic acid (Compound 152) (6)

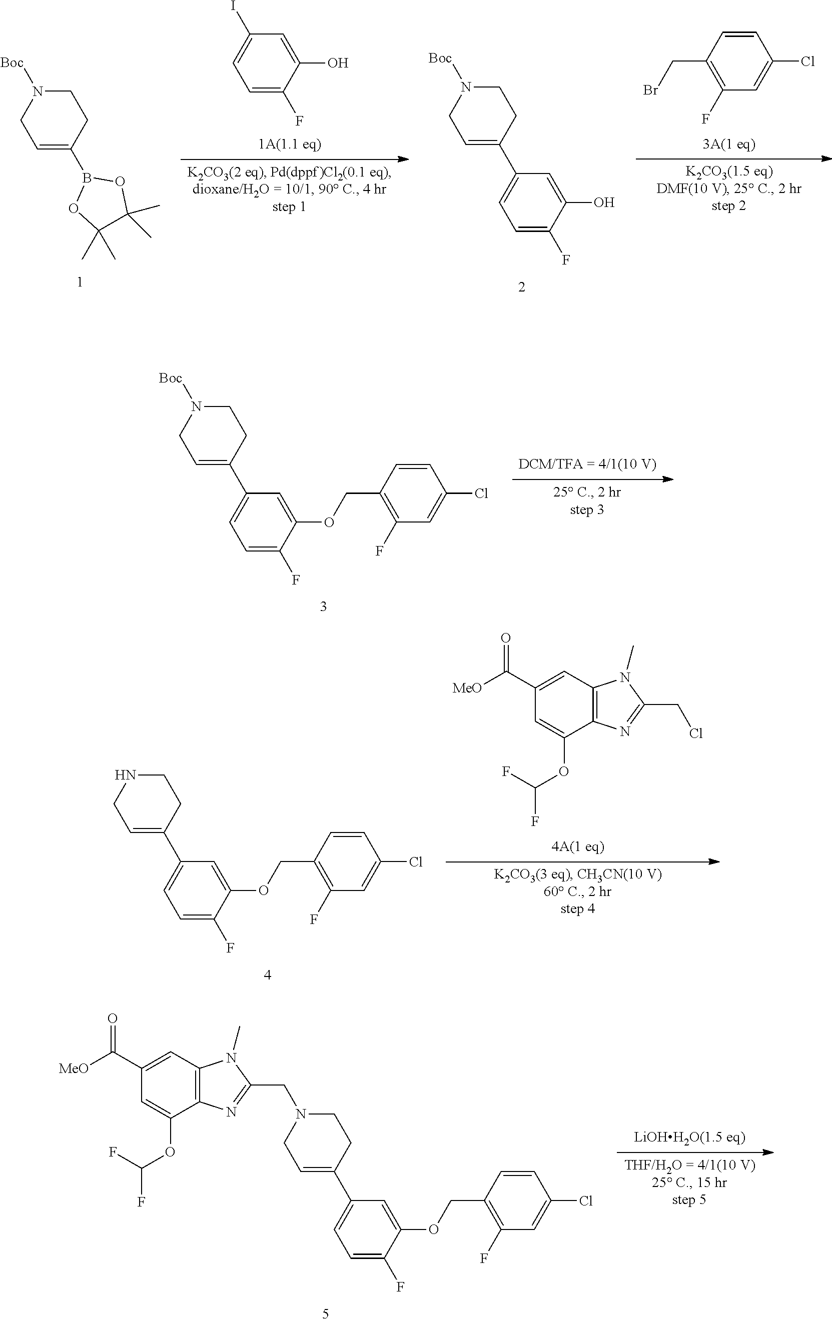

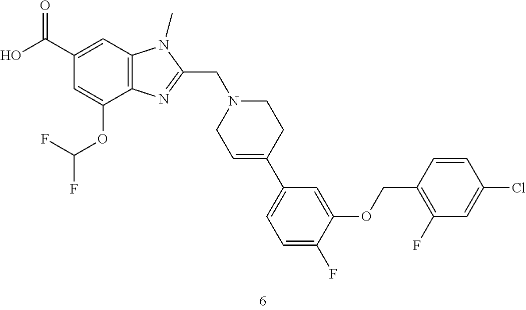 [SMILES: Cn1c(CN2CC=C(c3ccc(F)c(OCc4ccc(Cl)cc4F)c3)CC2)nc2c(OC(F)F)cc(C(=O)O)cc21]

### General Procedure for Preparation of Intermediate 2:

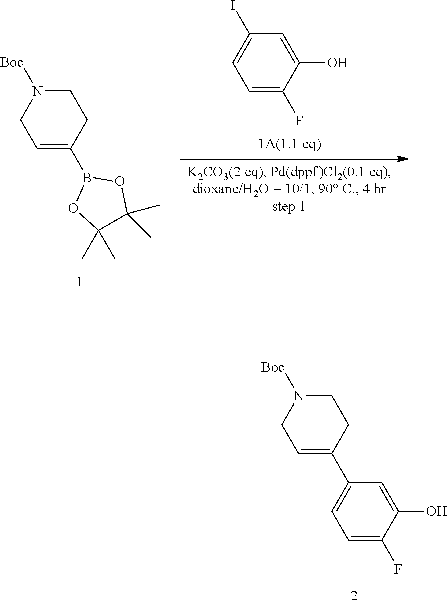

To a mixture of 1 (1.05 g, 3.40 mmol, 1 eq), 1A (889.01 mg, 3.74 mmol, 1.1 eq) and K₂CO₃ (938.63 mg, 6.79 mmol, 2 eq) in dioxane (10 mL) and H₂O (1 mL) was added Pd(dppf)Cl₂ (124.24 mg, 169.79 μmol, 0.05 eq) at 25° C. The mixture was de-gassed under reduced pressure and recharged with N₂. The mixture was stirred at 90° C. for 3 hours. LC-MS showed 1 was consumed completely and one main peak with desired mass was detected. The residue was poured into water (50 mL). The aqueous phase was extracted with ethyl acetate (20 mL*3). The combined organic phase was washed with saturated brine (50 mL*1), dried with anhydrous Na₂SO₄, filtered, and concentrated in vacuum. The residue was purified by column chromatography (SiO₂, PE/EtOAc=1/0 to 2/1). 2 (750 mg, 2.54 mmol, 74.84% yield) was obtained as a colorless oil.

LCMS: RT=0.793 min, MS cal.: 293.1, [M+H-t-Bu]⁺=238.0

HPLC: product: RT=2.413 min, purity: 99.38%

¹H NMR (400 MHz, MeOH-d4) δ=7.01-6.94 (m, 2H), 6.84 (ddd, J=2.4, 4.4, 8.4 Hz, 1H), 5.99 (br s, 1H), 4.03 (br s, 2H), 3.61 (br s, 2H), 2.46 (br d, J=1.6 Hz, 2H), 1.49 (s, 9H)

### General Procedure for Preparation of Intermediate 3:

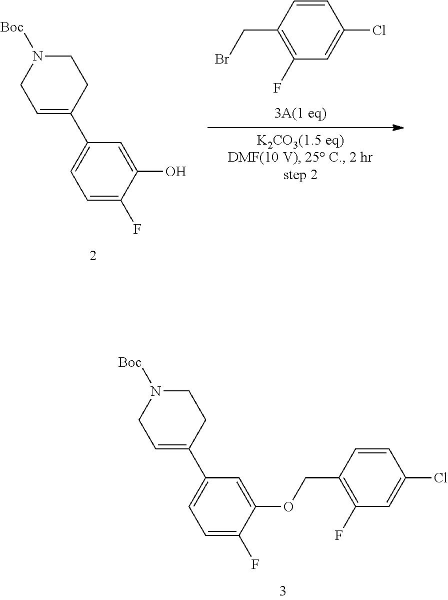

To a solution 2 (300 mg, 1.02 mmol, 1 eq) in DMF (3 mL) was added K₂CO₃ (212.02 mg, 1.53 mmol, 1.5 eq) and 3A (228.55 mg, 1.02 mmol, 1 eq). The mixture was stirred at 25° C. for 2 hr. LC-MS showed 2 was consumed completely and one main peak with desired mass was detected. The residue was poured into water (50 mL). The aqueous phase was extracted with ethyl acetate (20 mL*3). The combined organic phase was washed with saturated brine (50 mL*1), dried with anhydrous Na₂SO₄, filtered and concentrated in vacuum. The residue was purified by column chromatography (SiO₂, PE/EtOAc=1/0 to 1/1). 3 (440 mg, 964.00 μmol, 94.26% yield) was obtained as a yellow solid.

LCMS: RT=0.978 min, MS cal.: 435.1, [M+H−t-Bu]⁺=380.1

HPLC: product: RT=3.526 min, purity: 95.52%

### General Procedure for Preparation of Intermediate 4:

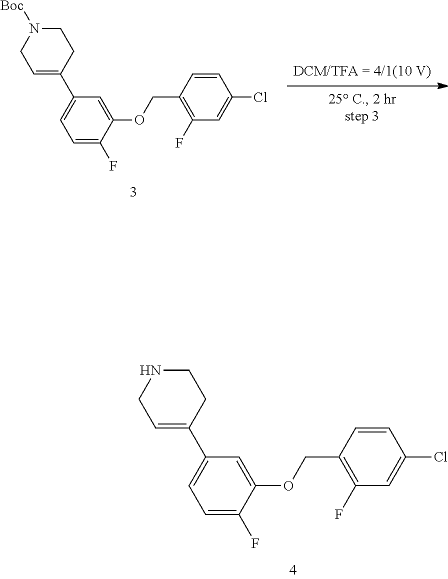

To a solution of 3 (400 mg, 917.66 μmol, 1 eq) in DCM (4 mL) was added TFA (1.54 g, 13.46 mmol, 1 mL, 14.67 eq). The mixture was stirred at 25° C. for 2 hr. LC-MS (ET83210-10-P1A) showed 3 was consumed completely and one main peak with desired mass was detected. The mixture was filtered and concentrated in vacuum. The crude product was used into the next step without further purification. 4 (225 mg, 643.96 μmol, 70.17% yield) was obtained as a yellow solid.

LCMS: RT=0.782 min, MS cal.: 335.1, [M+H]⁺=336.1

HPLC: product: RT=2.296 min, purity: 96.11%

¹H NMR (400 MHz, MeOH-d4) δ=7.53 (t, J=8.0 Hz, 1H), 7.27-7.22 (m, 2H), 7.18 (dd, J=2.0, 8.0 Hz, 1H), 7.10-6.99 (m, 2H), 6.13-6.07 (m, 1H), 5.19 (s, 2H), 3.51 (d, J=3.2 Hz, 2H), 3.11 (t, J=6.0 Hz, 2H), 2.54-2.44 (m, 2H)

### General Procedure for Preparation of Intermediate 5:

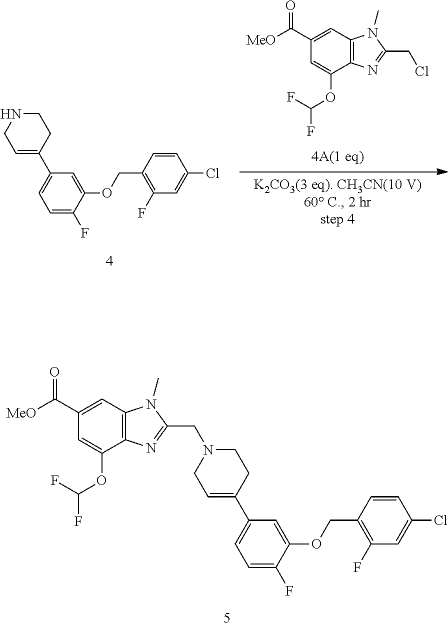 [SMILES: Fc1cc(Cl)ccc1COc1cc(C2=CCNCC2)ccc1F ; COC(=O)c1cc(OC(F)F)c2nc(CCl)n(C)c2c1 ; "4A(1 eq)" ; "K2CO3(3 eq). CH3CN(10 V)60° C., 2 hrstep 4" ; COC(=O)c1cc(OC(F)F)c2nc(CN3CC=C(c4ccc(F)c(OCc5ccc(Cl)cc5F)c4)CC3)n(C)c2c1]

To a solution of 4 (80 mg, 238.25 μmol, 1 eq) in ACN (1 mL) was added K₂CO₃ (98.78 mg, 714.76 μmol, 3 eq) 4A (72.59 mg, 238.25 μmol, 1 eq). The mixture was stirred at 60° C. for 2 hr. LC-MS (ET83210-14-P1B) showed 4 was consumed completely and one main peak with desired mass was detected. The mixture was filtered and concentrated in vacuum. The residue was purified by column chromatography (SiO₂, PE/EtOAc=1/0 to 1/1). 5 (140 mg, 207.22 μmol, 86.97% yield) was obtained as a yellow solid.

LCMS: RT=0.516 min, MS cal.: 603.2, [M+H]⁺=604.3

HPLC: product: RT=3.420 min, purity: 89.35%

¹H NMR (400 MHz, CHCl3-d) δ=7.99 (d, J=0.8 Hz, 1H), 7.73 (s, 1H), 7.31 (t, J=74.0 Hz, 1H), 7.52-7.46 (m, 1H), 7.20-7.16 (m, 1H), 7.15-7.10 (m, 2H), 7.08-7.00 (m, 2H), 6.98-6.92 (m, 1H), 5.98 (br s, 1H), 5.16 (s, 2H), 4.00 (s, 2H), 3.98 (d, J=2.8 Hz, 6H), 3.28 (br s, 2H), 2.87-2.78 (m, 2H), 2.51 (br s, 2H)

### General Procedure for Preparation of Compound 152 (6):

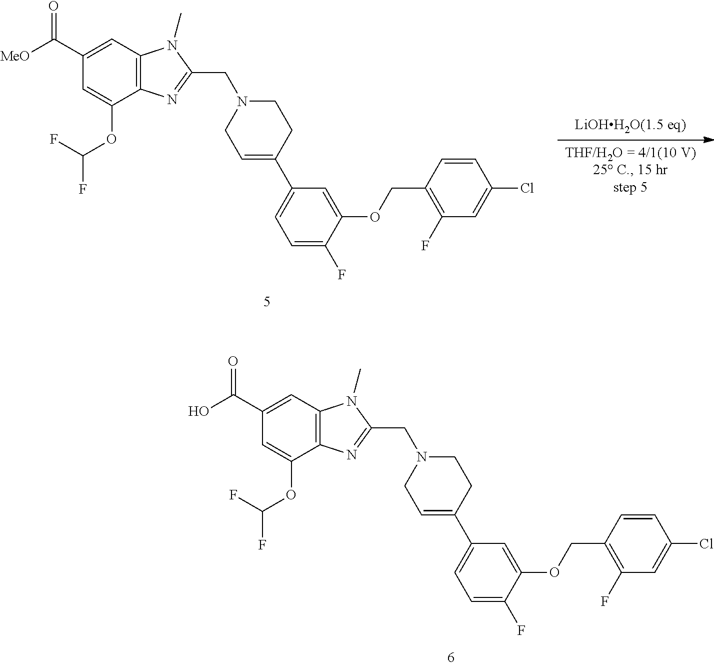

To a solution of 5 (130 mg, 215.24 μmol, 1 eq) in THF (1.4 mL) and H₂O (0.6 mL) was added LiOH·H₂O (13.55 mg, 322.85 μmol, 1.5 eq). The mixture was stirred at 25° C. for 16 hr. LC-MS showed 5 was consumed completely and one main peak with desired mass was detected. The mixture was filtered and concentrated in vacuum. The residue was purified by prep-HPLC (column: Waters Xbridge Prep OBD C₁₈ 150*40 mm*10 um; mobile phase: [H₂O (10 mM NH₄HCO₃)-ACN]; gradient: 30%-60% B over 8.0 min). 2-((4-(3-((4-Chloro-2-fluorobenzyl)oxy)-4-fluorophenyl)-3,6-dihydropyridin-1(2H)-yl)methyl)-4-(difluoromethoxy)-1-methyl-1H-benzo[d]imidazole-6-carboxylic acid (80 mg, 134.39 μmol, 62.44% yield) was obtained as a yellow solid.

LCMS: RT=2.840 min, MS cal.: 589.1, [M+H]⁺=590.2.

HPLC: RT=12.482 min, purity: 99.11%

¹H NMR (400 MHz, MeOH-d4) δ=8.11 (s, 1H), 7.69 (s, 1H), 7.52 (t, J=8.0 Hz, 1H), 7.24 (t, J=74.4 Hz, 1H), 7.28-7.22 (m, 2H), 7.20-7.16 (m, 1H), 7.09-6.98 (m, 2H), 6.07 (br s, 1H), 5.18 (s, 2H), 4.03 (s, 2H), 4.00 (s, 3H), 3.28 (br d, J=2.4 Hz, 2H), 2.87 (t, J=5.6 Hz, 2H), 2.55 (br s, 2H)

### Example A197—Preparation of Compound 153. 2-((4-(6-((4-Chloro-2,6-difluorobenzyl)oxy)pyridin-2-yl)piperidin-1-yl)methyl)-4-(difluoromethoxy)-1-methyl-1H-benzo[d]imidazole-6-carboxylic acid (Compound 153) (5)

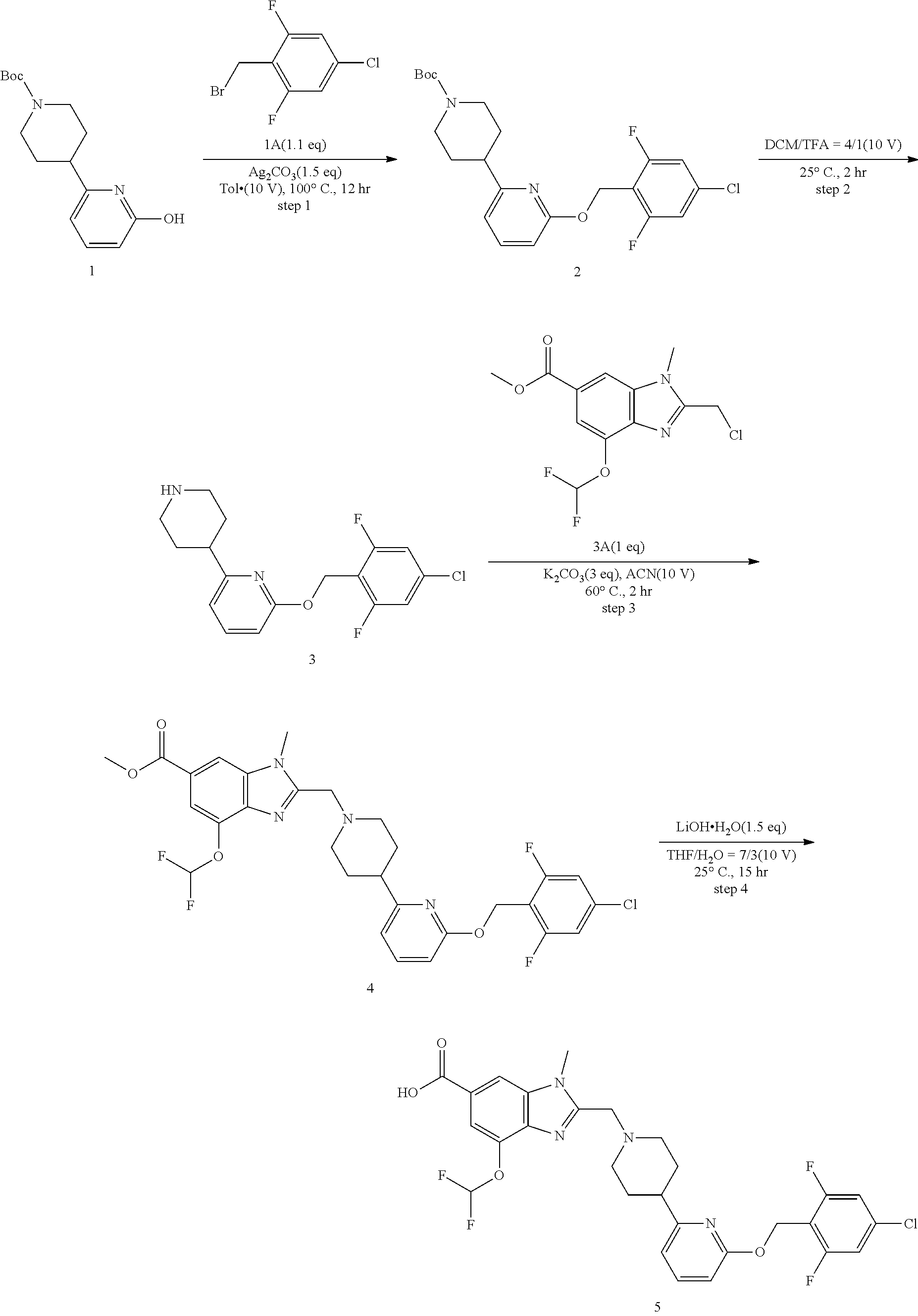

### General Procedure for Preparation of Intermediate 2:

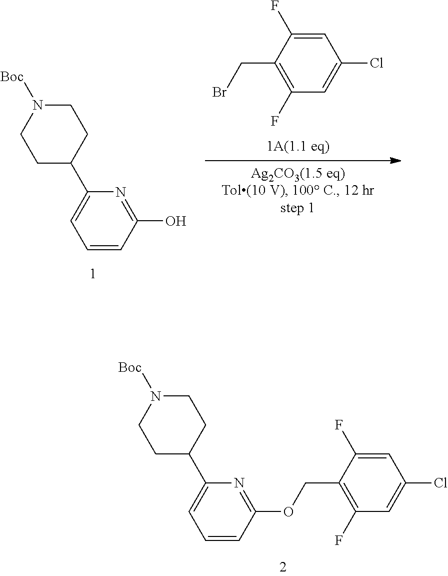

A mixture of 1 (270 mg, 970.02 μmol, 1 eq), 1A (257.64 mg, 1.07 mmol, 1.1 eq), Ag₂CO₃ (401.22 mg, 1.46 mmol, 66.02 μL, 1.5 eq) in Toluene (3 mL) was degassed and purged with N₂ for 3 times, and then the mixture was stirred at 100° C. for 12 hr under N₂ atmosphere. The color changes to brown. LC-MS showed 1 was consumed completely and one main peak with desired mass was detected. The residue was poured into water (50 mL). The aqueous phase was extracted with ethyl acetate (20 mL*3). The combined organic phase was washed with saturated brine (50 mL*1), dried with anhydrous Na₂SO₄, filtered and concentrated in vacuum. The residue was purified by column chromatography (SiO₂, PE/EtOAc=10/1 to 0/1). 2 (275 mg, 591.49 μmol, 60.98% yield) was obtained as a colorless oil.

LCMS: RT=1.012 min, MS cal.: 438.2, [M+H]⁺=439.2

HPLC: product: RT=3.627 min, purity: 94.45%

¹H NMR (400 MHz, MeOH-d4) δ=7.56 (t, J=8.0 Hz, 1H), 7.15-7.12 (m, 1H), 7.12-7.09 (m, 1H), 6.82 (d, J=7.2 Hz, 1H), 6.58 (d, J=8.0 Hz, 1H), 5.40 (s, 2H), 4.19 (br d, J=13.6 Hz, 2H), 2.98-2.75 (m, 3H), 1.86-1.78 (m, 2H), 1.78-1.66 (m, 2H), 1.49 (s, 9H)

### General Procedure for Preparation of Intermediate 3:

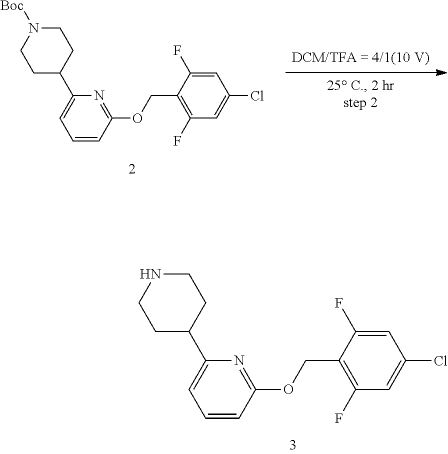

To a solution 2 (270 mg, 615.18 μmol, 1 eq) in DCM (2.8 mL) was added TFA (1.09 g, 9.60 mmol, 713.21 μL, 15.61 eq). The mixture was stirred at 25° C. for 2 hr. LC-MS showed 2 was consumed completely and one main peak with desired mass was detected. The mixture was filtered and concentrated in vacuum. The crude product was used into the next step without further purification. 3 (220 mg, crude) was obtained as a yellow solid.

LCMS: RT=0.743 min, MS cal.: 338.1, [M+H]⁺=339.1

HPLC: product: RT=2.121 min, purity: 94.74%

¹H NMR (400 MHz, MeOH-d4) δ=7.59 (t, J=8.0 Hz, 1H), 7.18-7.15 (m, 1H), 7.15-7.12 (m, 1H), 6.84 (d, J=7.2 Hz, 1H), 6.59 (d, J=8.0 Hz, 1H), 5.40 (s, 2H), 3.22 (br d, J=12.8 Hz, 2H), 2.86-2.81 (m, 2H), 2.79 (br d, J=2.8 Hz, 1H), 1.98-1.91 (m, 2H), 1.90-1.79 (m, 2H)

### General Procedure for Preparation of Intermediate 4:

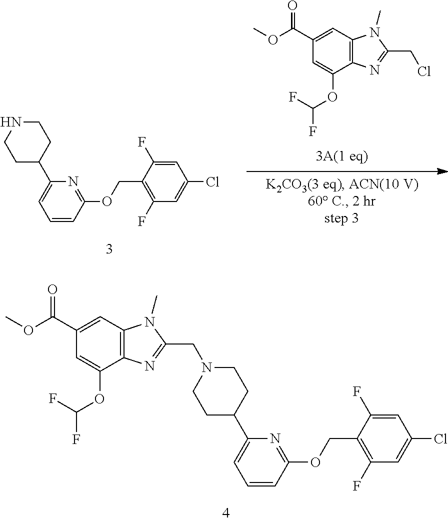 [SMILES: Fc1cc(Cl)cc(F)c1COc1cccc(C2CCNCC2)n1 ; COC(=O)c1cc(OC(F)F)c2nc(CCl)n(C)c2c1 ; "3A(1 eq)" ; "K2CO3(3 eq), ACN(10 V)60° C., 2 hrstep 3" ; COC(=O)c1cc(OC(F)F)c2nc(CN3CCC(c4cccc(OCc5c(F)cc(Cl)cc5F)n4)CC3)n(C)c2c1]

To a solution of 3 (86 mg, 253.85 μmol, 1 eq) in ACN (1 mL) was added K₂CO₃ (105.25 mg, 761.56 μmol, 3 eq) and 3A (77.34 mg, 253.85 μmol, 1 eq). The mixture was de-gassed under reduced pressure and recharged with N₂. The mixture was stirred at 60° C. for 1 hr. LC-MS showed 3 was consumed completely and one main peak with desired mass was detected. The mixture was filtered and concentrated in vacuum. The residue was purified by prep-TLC (SiO₂, Petroleum ether:Ethyl acetate=2:1, Rf=0.40). 4 (130 mg, 206.52 μmol, 81.36% yield) was obtained as a yellow solid.

LCMS: RT=0.498 min, MS cal.: 606.2, [M+H]⁺=607.3

HPLC: product: RT=3.561 min, purity: 96.43%

¹H NMR (400 MHz, MeOH-d4) δ=8.13 (d, J=1.2 Hz, 1H), 7.67 (s, 1H), 7.58-7.53 (m, 1H), 7.26 (t, J=74.4 Hz, 1H), 7.14.-7.13 (m, 1H), 7.11-7.08 (m, 1H), 6.82 (d, J=7.6 Hz, 1H), 6.56 (d, J=8.0 Hz, 1H), 5.39 (s, 2H), 4.59 (s, 1H), 4.04 (s, 3H), 3.96 (s, 3H), 3.92 (s, 2H), 3.06-3.00 (m, 2H), 2.70-2.61 (m, 1H), 2.38-2.29 (m, 2H), 1.93-1.86 (m, 4H)

### General Procedure for Preparation of Compound 153 (5):

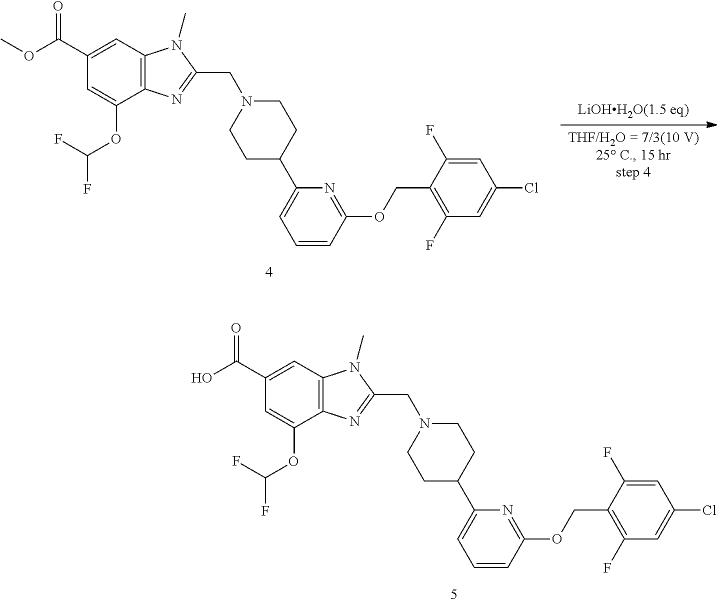

To a solution of 4 (120 mg, 197.70 μmol, 1 eq) in THF (1.4 mL) and H₂O (0.6 mL) was added LiOH·H₂O (12.44 mg, 296.54 μmol, 1.5 eq). The mixture was stirred at 25° C. for 15 hr. LC-MS showed 4 was consumed completely and one main peak with desired mass was detected. The mixture was filtered and concentrated in vacuum. The residue was purified by prep-HPLC (column: Waters Xbridge Prep OBD C₁₈ 150*40 mm*10 um; mobile phase: [H₂O (10 mM NH₄HCO₃)-ACN]; gradient: 30%-60% B over 8.0 min). 2-((4-(6-((4-Chloro-2,6-difluorobenzyl)oxy)pyridin-2-yl)piperidin-1-yl)methyl)-4-(difluoromethoxy)-1-methyl-1H-benzo[d]imidazole-6-carboxylic acid (85 mg, 143.35 μmol, 72.51% yield) was obtained as a white solid.

LCMS: RT=2.837 min, MS cal.: 592.2 [M+H]⁺=593.2

HPLC: RT=12.172 min, purity: 100.00%

¹H NMR (400 MHz, MeOH-d4) δ=8.10 (d, J=0.8 Hz, 1H), 7.68 (s, 1H), 7.56 (t, J=8.0 Hz, 1H), 7.25 (t, J=74.4 Hz, 1H), 7.13-7.11 (m, 2H), 6.84 (d, J=7.2 Hz, 1H), 6.57 (d, J=8.0 Hz, 1H), 5.38 (s, 2H), 4.07-3.98 (m, 5H), 3.13 (d, J=11.2 Hz, 2H), 2.76-2.65 (m, 1H), 2.50-2.40 (m, 2H), 2.01-1.90 (m, 4H)

### Example A198—Preparation of Compound 154. 4-(Difluoromethoxy)-2-((4-(6-((2-methoxy-4-(trifluoromethyl)benzyl)oxy)pyridin-2-yl)piperidin-1-yl)methyl)-1-methyl-1H-benzo[d]imidazole-6-carboxylic acid (Compound 154) (5)

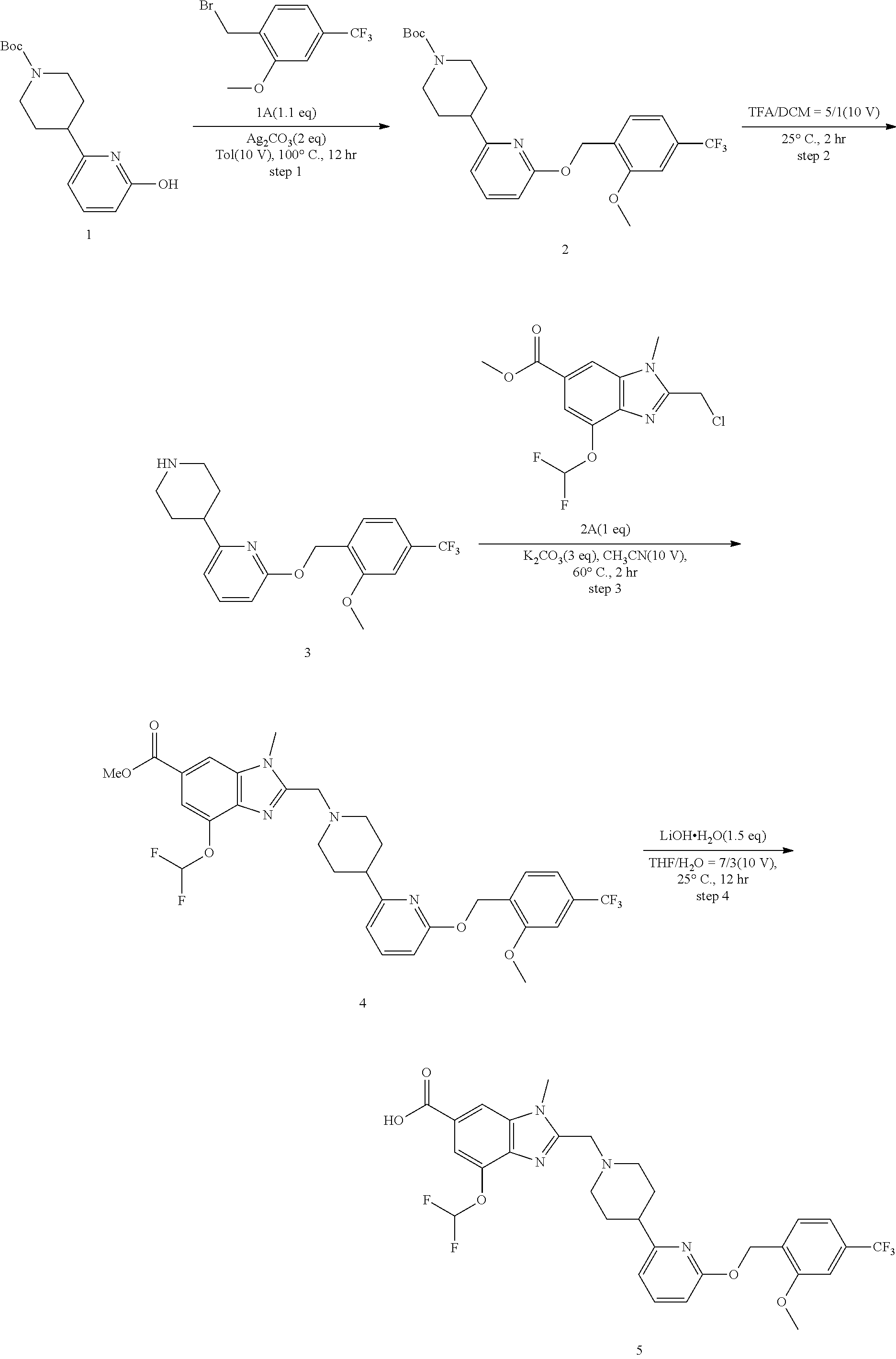

### General Procedure for Preparation of Intermediate 2:

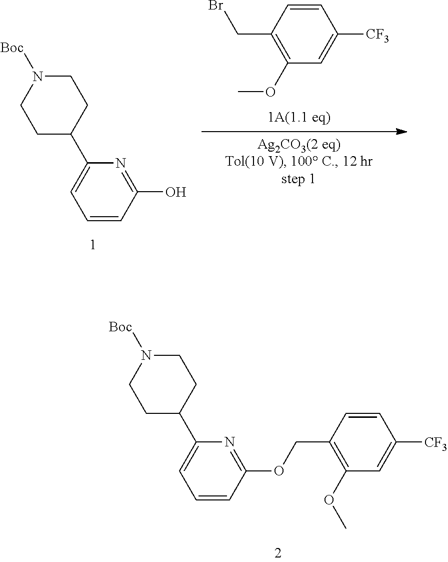

A mixture of 1 (300 mg, 1.08 mmol, 1 eq), 1A (318.99 mg, 1.19 mmol, 1.1 eq) and Ag₂CO₃ (594.39 mg, 2.16 mmol, 97.81 μL, 2 eq) in Toluene (3 mL) was degassed and purged with N₂ for 3 times. The mixture was stirred 100° C. for 12 hours. The color changes to brown. TLC (SiO₂, PE:EA=2:1, R_{f}=0.50) showed the reaction was completed. The reaction mixture was quenched by addition water 10 mL at 25° C., and then diluted with EA 5 mL and extracted with EA 15 mL (5 mL*3). The combined organic layers were washed with brine (10 mL), dried over Na₂SO₄, filtered and concentrated under reduced pressure to give a residue. The residue was purified or by prep-TLC (SiO₂, PE:EA=2:1, R_{f}=0.45). 2 (300 mg, 637.95 μmol, 59.19% yield, 99.2% purity) was obtained as a light orange solid.

HPLC: RT=3.565 min, purity: 99.34%

¹H NMR (400 MHz, MeOH-d4) δ=7.59 (t, J=7.8 Hz, 1H), 7.52 (d, J=8.0 Hz, 1H), 7.24-7.18 (m, 2H), 6.80 (d, J=7.2 Hz, 1H), 6.68 (d, J=8.4 Hz, 1H), 5.43 (s, 2H), 4.18-4.07 (m, 2H), 3.94 (s, 3H), 2.86 (br s, 2H), 2.76 (tt, J=3.6, 11.6 Hz, 1H), 1.77 (br d, J=12.0 Hz, 2H), 1.62 (br d, J=12.8 Hz, 2H), 1.48 (s, 9H)

### General Procedure for Preparation of Intermediate 3:

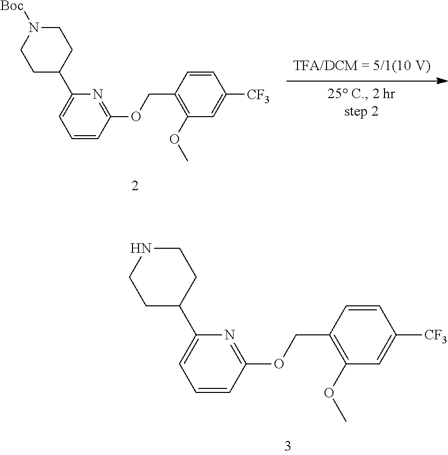

A mixture of 2 (300 mg, 643.10 μmol, 1 eq) in DCM (3 mL) and TFA (0.6 mL) was stirred at 25° C. for 1 hour. The color changes to orange. LCMS showed 93% of desired Compound 2 was detected. The reaction mixture was quenched by addition Sat. NaHCO₃30 mL at 25° C., and then diluted with EA 15 mL and extracted with EA 45 mL (15 mL*3). The combined organic layers were washed with brine (30 mL), dried over Na₂SO₄, filtered and concentrated under reduced pressure to give a residue. The mixture was used for the next step directly without purification. 3 (225 mg, 569.91 μmol, 88.62% yield, 92.8% purity) was obtained as an orange solid.

LCMS (Compound 3): RT=0.737 min, MS cal.: 366.2, [M+H]⁺=367.2

HPLC: RT=2.302 min, purity: 92.87%

¹H NMR (400 MHz, MeOH-d4) δ=7.68-7.53 (m, 2H), 7.30-7.18 (m, 2H), 6.84 (d, J=7.2 Hz, 1H), 6.70 (d, J=8.4 Hz, 1H), 5.45 (s, 2H), 3.94 (s, 3H), 3.20 (br d, J=12.8 Hz, 2H), 2.89-2.73 (m, 3H), 1.96-1.87 (m, 2H), 1.86-1.74 (m, 2H)

### General Procedure for Preparation of Intermediate 4:

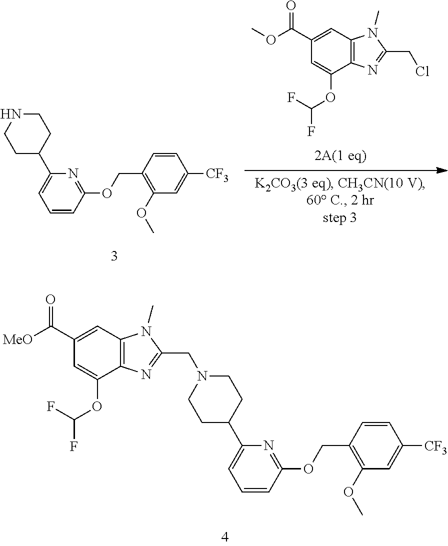 [SMILES: COc1cc(C(F)(F)F)ccc1COc1cccc(C2CCNCC2)n1 ; COC(=O)c1cc(OC(F)F)c2nc(CCl)n(C)c2c1 ; "2A(1 eq)" ; "K2CO3(3 eq), CH3CN(10 V),60° C., 2 hrstep 3" ; COC(=O)c1cc(OC(F)F)c2nc(CN3CCC(c4cccc(OCc5ccc(C(F)(F)F)cc5OC)n4)CC3)n(C)c2c1]

A mixture of 3 (100 mg, 272.94 μmol, 1 eq), 2A (83.16 mg, 272.94 μmol, 1 eq) and K₂CO₃ (113.17 mg, 818.83 μmol, 3 eq) in ACN (1 mL) was degassed and purged with N₂ for 3 times. The mixture was stirred 60° C. for 2 hours. The color changes to brown. TLC (SiO₂, PE:EA=2:1, R_{f}=0.50) showed the reaction was completed. The mixture was filtered and concentrated in vacuum. The residue was purified by prep-TLC (SiO₂, PE:EA=2:1, R_{f}=0.45). 4 (122 mg, 185.52 μmol, 67.97% yield, 96.5% purity) was obtained as an orange solid.

LCMS: RT=0.531 min, MS cal.: 634.2, [M+H]⁺=635.3

### General Procedure for Preparation of Compound 154 (5):

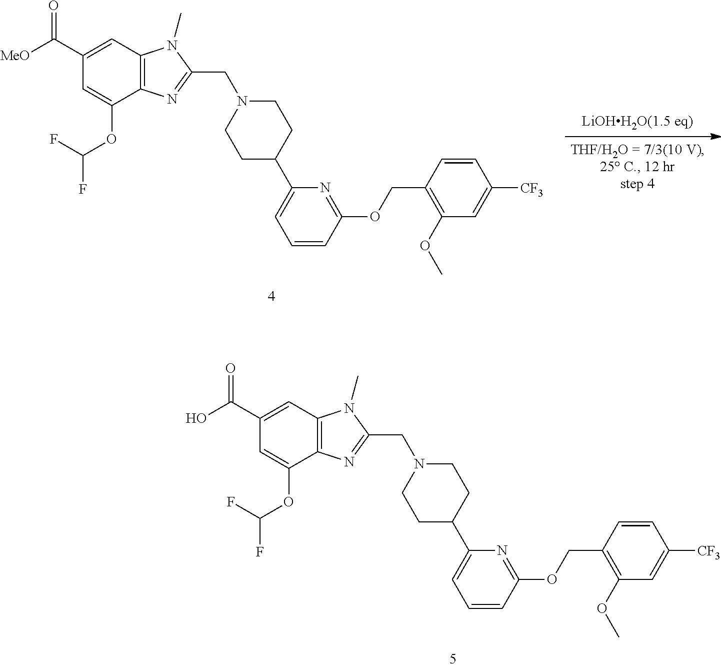

A mixture of 4 (122 mg, 192.25 μmol, 1 eq) in THF (1.4 mL) and H₂O (0.6 mL) was added LiOH·H₂O (12.10 mg, 288.37 μmol, 1.5 eq). The mixture was stirred at 25° C. for 12 hours. The color changes to brown. LCMS showed 86% of desired product was detected. The mixture was concentrated in vacuum. The residue was purified by prep-HPLC (column: Waters Xbridge Prep OBD C18 150*40 mm*10 um; mobile phase: [H₂O (10 mM NH₄HCO₃)-ACN]; gradient: 30%-60% B over 8.0 min). 4-(Difluoromethoxy)-2-((4-(6-((2-methoxy-4-(trifluoromethyl)benzyl)oxy)pyridin-2-yl)piperidin-1-yl)methyl)-1-methyl-1H-benzo[d]imidazole-6-carboxylic acid (79 mg, 127.02 μmol, 66.07% yield, 99.78% purity) was obtained as a white solid.

LCMS: RT=2.879 min, MS cal.: 620.2, [M+H]⁺=621.2

HPLC: RT=12.582 min, purity: 99.78%

¹H NMR (400 MHz, MeOH-d4) δ=8.10 (s, 1H), 7.68 (s, 1H), 7.59 (t, J=8.0 Hz, 1H), 7.55 (d, J=8.4 Hz, 1H), 7.26 (t, J=74 Hz, 1H), 7.24-7.20 (m, 2H), 6.83 (d, J=7.2 Hz, 1H), 6.68 (d, J=8.4 Hz, 1H), 5.43 (s, 2H), 4.00 (s, 5H), 3.93 (s, 3H), 3.12 (d, J=11.6 Hz, 2H), 2.74-2.61 (m, 1H), 2.52-2.37 (m, 2H), 1.95-1.83 (m, 4H)

### Example A199—Preparation of Compound 155. 2-((6-((4-Chloro-2-fluorobenzyl)oxy)-3′,6′-dihydro-[2,4′-bipyridin]-1′(2′H)-yl)methyl)-4-(difluoromethoxy)-1-methyl-1H-benzo[d]imidazole-6-carboxylic acid (Compound 155) (6)

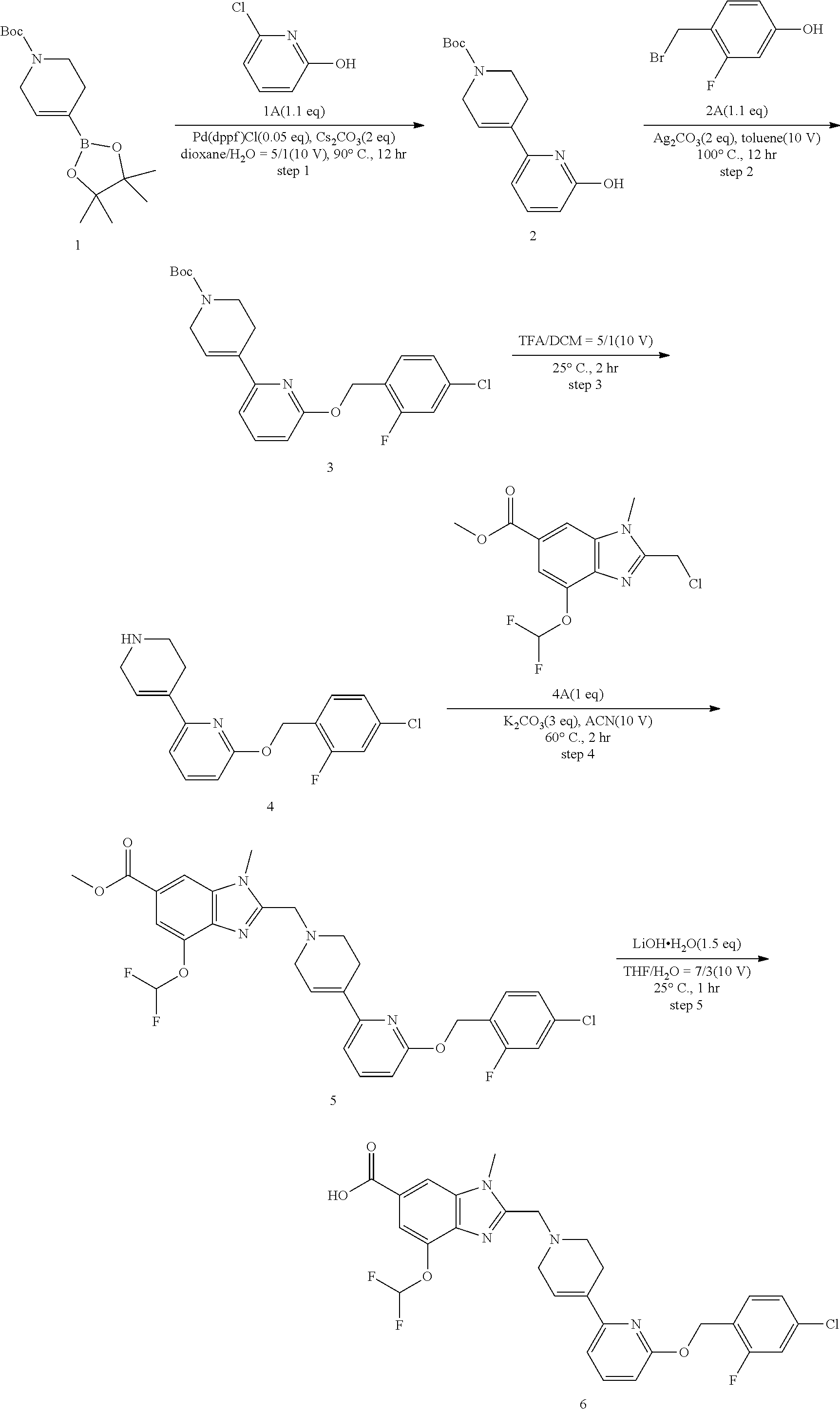

### General Procedure for Preparation of Intermediate 2:

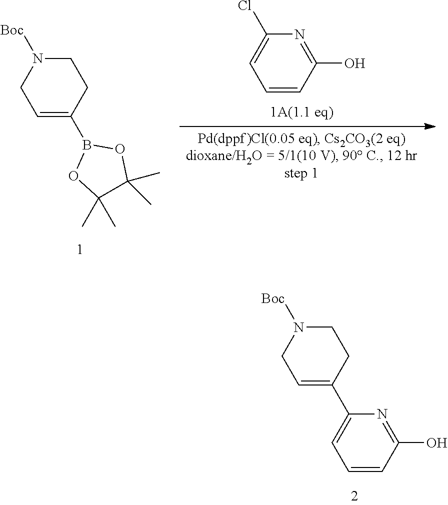

To a mixture of 1 in dioxane (40 mL) and H₂O (4 mL) was added 1A and Cs₂CO₃ (8.43 g, 25.87 mmol, 2 eq). The mixture was degassed and purged with N₂ for 3 times. Then Pd(dppf)Cl₂ (473.28 mg, 646.81 μmol, 0.05 eq) was added. The mixture was degassed and purged with N₂ for another 3 times. The mixture was stirred at 90° C. for 12 hours. The color changes to black. TLC (SiO₂, PE:EA=1:2, R_{f}=0.20) showed the reaction was completed. The mixture was concentrated in vacuum. The residue was purified by column chromatography (SiO₂, Petroleum ether/Ethyl acetate=1/4 to 0/1). 2 (1.2 g, 3.52 mmol, 27.19% yield, 81% purity) was obtained as a brown solid.

HPLC: RT=1.605 min, purity: 58.18%

¹H NMR (400 MHz, MeOH-d4) δ=7.55 (dd, J=7.2, 8.8 Hz, 1H), 6.44 (d, J=8.8 Hz, 1H), 6.40 (d, J=6.8 Hz, 1H), 6.35 (br s, 1H), 4.11 (br s, 2H), 3.62 (br t, J=5.2 Hz, 2H), 2.47 (br d, J=0.8 Hz, 2H), 1.49 (s, 9H)

### General Procedure for Preparation of Intermediate 3:

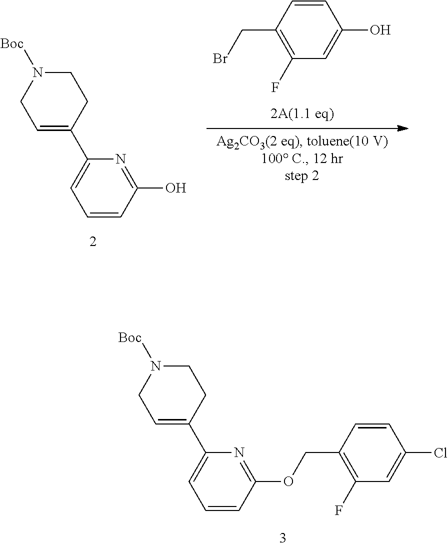

To a mixture of 2 (500 mg, 1.81 mmol, 1 eq), 2A (444.79 mg, 1.99 mmol, 1.1 eq) and Ag₂CO₃ (997.88 mg, 3.62 mmol, 164.21 μL, 2 eq) in Toluene (5 mL) was degassed and purged with N₂ for 3 times. The mixture was stirred 100° C. for 12 hours. The color changes to brown. TLC (SiO₂, PE:EA=5:1, R_{f}=0.50) showed the reaction was completed. The reaction mixture was quenched by addition water 20 mL at 25° C., and then diluted with EA 10 mL and extracted with EA 45 mL (15 mL*3). The combined organic layers were washed with brine (30 mL), dried over Na₂SO₄, filtered, and concentrated under reduced pressure to give a residue. The residue was purified by prep-TLC (SiO₂, PE:EA=5:1, R_{f}=0.45). 3 (580 mg, 1.37 mmol, 75.60% yield, 98.8% purity) was obtained as light-yellow oil.

HPLC: RT=3.623 min, purity: 98.78%

¹H NMR (400 MHz, MeOH-d4) δ=7.63 (t, J=8.0 Hz, 1H), 7.50 (t, J=8.4 Hz, 1H), 7.28-7.13 (m, 2H), 7.08 (d, J=8.8 Hz, 1H), 6.69 (d, J=8.0 Hz, 2H), 5.44 (s, 2H), 4.10 (br s, 2H), 3.67-3.58 (m, 2H), 2.58 (br s, 2H), 1.50 (s, 9H)

### General Procedure for Preparation of Intermediate 4:

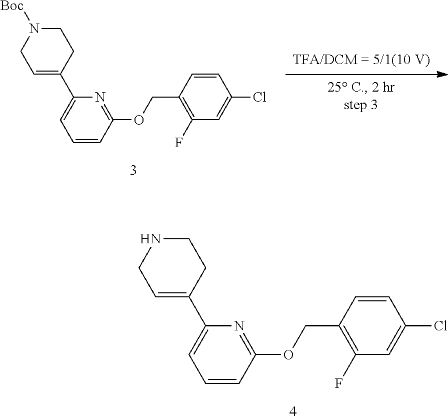

A mixture of 3 (250 mg, 596.82 μmol, 1 eq) in DCM (2.5 mL) and TFA (0.5 mL) was stirred at 25° C. for 1 hour. The color changes to yellow. TLC (SiO₂, PE:EA=2:1, R_{f}=0.40) showed the reaction was completed. The reaction mixture was quenched by addition Sat. NaHCO₃30 mL at 25° C., and then diluted with EA 15 mL and extracted with EA 45 mL (15 mL*3). The combined organic layers were washed with brine (30 mL), dried over Na₂SO₄, filtered, and concentrated under reduced pressure to give a residue. The residue was purified by prep-TLC (SiO₂, PE:EA=2:1, R_{f}=0.35). 4 (135 mg, 389.20 μmol, 65.21% yield, 91.9% purity) was obtained as a yellow solid.

LCMS: RT=0.806 min, MS cal.: 318.1, [M+H]+=319.1

¹H NMR (400 MHz, MeOH-d4) δ=7.63 (t, J=7.6 Hz, 1H), 7.49 (t, J=8.0 Hz, 1H), 7.25-7.16 (m, 2H), 7.07 (d, J=7.2 Hz, 1H), 6.77 (br s, 1H), 6.69 (d, J=8.0 Hz, 1H), 5.44 (s, 2H), 3.56 (br d, J=2.8 Hz, 2H), 3.11 (t, J=6.0 Hz, 2H), 2.59 (br d, J=2.0 Hz, 2H)

### General Procedure for Preparation of Intermediate 5:

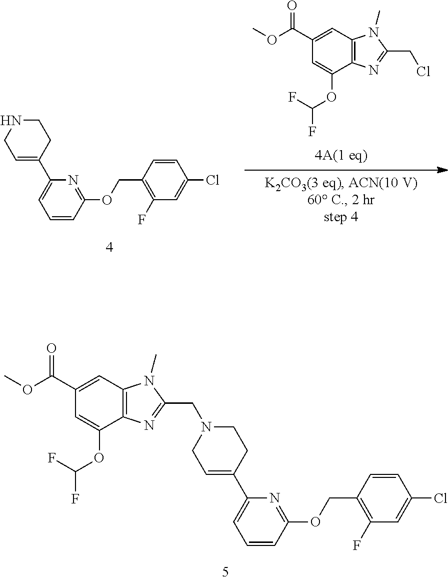 [SMILES: Fc1cc(Cl)ccc1COc1cccc(C2=CCNCC2)n1 ; COC(=O)c1cc(OC(F)F)c2nc(CCl)n(C)c2c1 ; "4A(1 eq)" ; "K2CO3(3 eq), ACN(10 V)60° C., 2 hrstep 4" ; COC(=O)c1cc(OC(F)F)c2nc(CN3CC=C(c4cccc(OCc5ccc(Cl)cc5F)n4)CC3)n(C)c2c1]

A mixture of 4 (100 mg, 313.70 μmol, 1 eq), 4A (95.58 mg, 313.70 μmol, 1 eq) and K₂CO₃ (130.07 mg, 941.11 μmol, 3 eq) in ACN (1 mL) was degassed and purged with N₂ for 3 times. The mixture was stirred 60° C. for 2 hours. The color changes to brown. TLC (SiO₂, PE:EA=2:1, R_{f}=0.60) showed the reaction was completed. The mixture was filtered and concentrated in vacuum. The residue was purified by prep-TLC (SiO₂, PE:EA=2:1, R_{f}=0.55). 5 (150 mg, 252.35 μmol, 80.44% yield, 98.75% purity) was obtained as a yellow solid.

LCMS: RT=0.987 min, MS cal.: 586.1, [M+H]⁺=587.2

¹H NMR (400 MHz, MeOH-d4) δ=8.13 (d, J=1.2 Hz, 1H), 7.68 (s, 1H), 7.61 (t, J=7.6 Hz, 1H), 7.50-7.10 (m, 1H), 7.49-7.45 (m, 1H), 7.23-7.16 (m, 2H), 7.04 (d, J=7.2 Hz, 1H), 6.72 (br s, 1H), 6.67 (d, J=8.0 Hz, 1H), 5.42 (s, 2H), 4.02 (s, 2H), 4.01 (s, 3H), 3.96 (s, 3H), 3.38 (br d, 2H), 2.84 (t, J=5.6 Hz, 2H), 2.62 (br s, 2H)

### General Procedure for Preparation of Compound 155 (6):

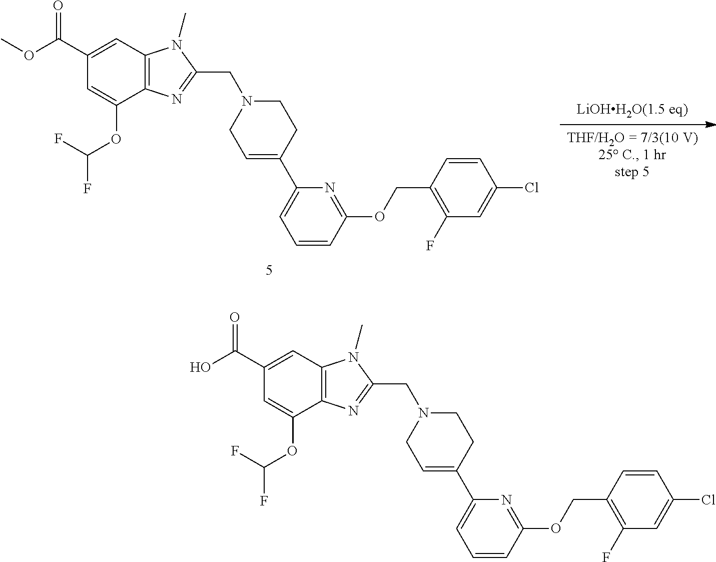

A mixture of 5 (120 mg, 204.43 μmol, 1 eq) in THF (1.4 mL) and H₂O (0.6 mL) was added LiOH·H₂O (12.87 mg, 306.65 μmol, 1.5 eq). The mixture was stirred at 25° C. for 12 hours. The color changes to brown. LCMS showed 81% of desired product was detected. The mixture was concentrated in vacuum. The residue was purified by prep-HPLC (column: Waters Xbridge Prep OBD C₁₈ 150*40 mm*10 um; mobile phase: [H₂O (10 mM NH₄HCO₃)-ACN]; gradient: 30%-60% B over 8.0 min). 2-((6-((4-Chloro-2-fluorobenzyl)oxy)-3′,6′-dihydro-[2,4′-bipyridin]-1′(2′H)-yl)methyl)-4-(difluoromethoxy)-1-methyl-1H-benzo[d]imidazole-6-carboxylic acid (78 mg, 135.59 μmol, 66.32% yield, 99.6% purity) was obtained as a white solid.

LCMS: RT=2.816 min, MS cal.: 572.1, [M+H]⁺=573.2

HPLC: RT=12.213 min, purity: 99.61%

¹H NMR (400 MHz, MeOH-d4) δ=8.10 (s, 1H), 7.69 (s, 1H), 7.61 (t, J=7.6 Hz, 1H), 7.48 (t, J=8.4 Hz, 1H), 7.45-7.08 (m, 1H), 7.23-7.15 (m, 2H), 7.04 (d, J=7.6 Hz, 1H), 6.72 (br s, 1H), 6.67 (d, J=8.0 Hz, 1H), 5.42 (s, 2H), 4.05 (s, 2H), 4.00 (s, 3H), 3.33 (br d, J=2.8 Hz, 2H), 2.87 (t, J=5.6 Hz, 2H), 2.64 (br d, J=1.6 Hz, 2H)

### Example A200—Preparation of Compound 156. 2-((4-(6-((4-Cyano-2-methoxybenzyl)oxy)pyridin-2-yl)piperidin-1-yl)methyl)-4-(difluoromethoxy)-1-methyl-1H-benzo[d]imidazole-6-carboxylic acid (Compound 156) (5)

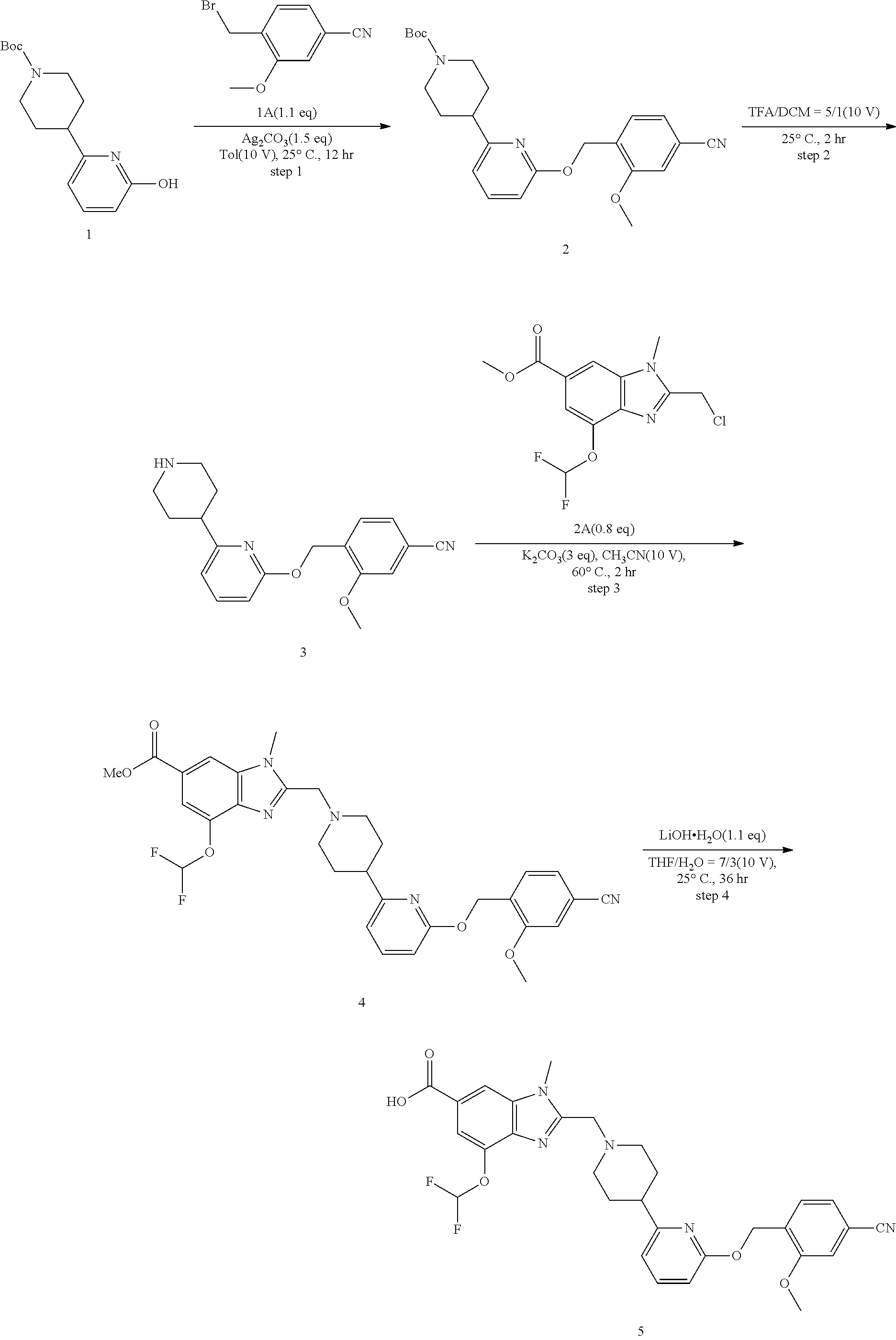

### General Procedure for Preparation of Intermediate 2:

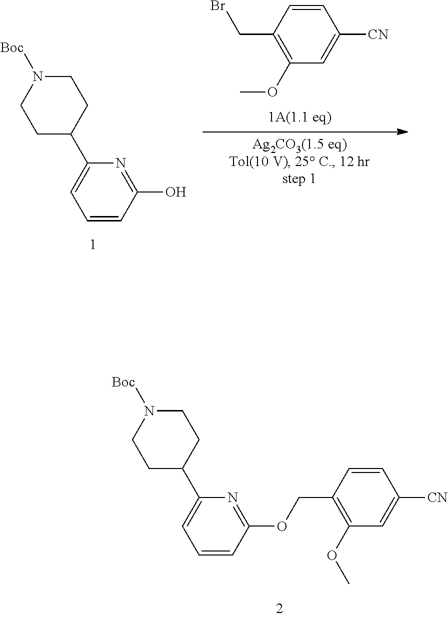

A mixture of 1 (300 mg, 1.08 mmol, 1 eq), 1A (268.02 mg, 1.19 mmol, 1.1 eq), Ag₂CO₃ (594.39 mg, 2.16 mmol, 97.81 μL, 2 eq) in toluene (3 mL) was degassed and purged with N₂ for 3 times, and then the mixture was stirred at 100° C. for 16 hr under N₂ atmosphere. LC-MS (ET83164-4-PIA) showed several new peaks were shown on LC-MS and 25% of desired Compound was detected showed 1 was consumed completely and one main peak with or desired mass was detected. The mixture filtered and concentrated in vacuum. The residue was purified by prep-TLC (SiO₂, PE:EtOAc=10:1). 2 (320 mg, 701.95 μmol, 65.13% yield) was obtained as a white solid.

LCMS: RT=0.632 min, MS cal.: 423.2, [M−(t-Bu)+H]⁺=368.2

¹H NMR (400 MHz, DMSO-d6) δ=7.65 (t, J=7.6 Hz, 1H) 7.48-7.53 (m, 2H) 7.38-7.45 (m, 1H) 6.87 (d, J=7.2 Hz, 1H) 6.73 (d, J=8.0 Hz, 1H) 5.37 (s, 2H) 3.95-4.09 (m, 2H) 3.89 (s, 3H) 2.68-2.88 (m, 3H) 1.72 (br d, J=12.8 Hz, 2H) 1.44-1.54 (m, 2H) 1.41 (s, 9H)

### General Procedure for Preparation of Intermediate 3:

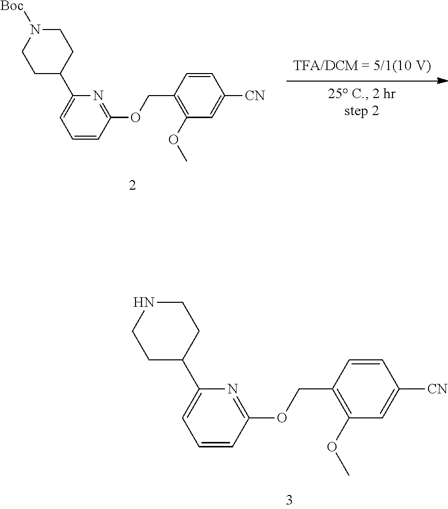

To a mixture of 2 (160 mg, 377.80 μmol, 1 eq) in DCM (2.5 mL) was added TFA (767.50 mg, 6.73 mmol, 500.00 μL, 17.82 eq). The mixture was stirred at 25° C. for 2 hours. LC-MS showed 94% of desired Compound was detected showed 2 was consumed completely and one main peak with or desired mass was detected. The mixture was concentrated in vacuum. The mixture was added 10 mL DCM. Then the reaction mixture was quenched by NaHCO₃ solution to pH=8. The aqueous phase was extracted with DCM (10 mL*3). The combined organic phase was washed with brine (10 mL*3), dried with anhydrous Na₂SO₄, filtered, and concentrated in vacuum. The combined filtrates were concentrated to dryness to give product. 3 (240 mg, crude) was obtained as a white solid.

LCMS: RT=0.375 min, MS cal.: 323.2, [M+H]⁺=324.1

¹H NMR (400 MHz, MeOH-d4) δ=7.63 (t, J=7.6 Hz, 1H) 7.54 (d, J=7.6 Hz, 1H) 7.25-7.38 (m, 2H) 6.86 (d, J=7.2 Hz, 1H) 6.73 (d, J=8.4 Hz, 1H) 5.46 (s, 2H) 3.94 (s, 3H) 3.27 (br s, 2H) 2.79-2.97 (m, 3H) 1.93-2.02 (m, 2H) 1.79-1.92 (m, 2H)

### General Procedure for Preparation of Intermediate 4:

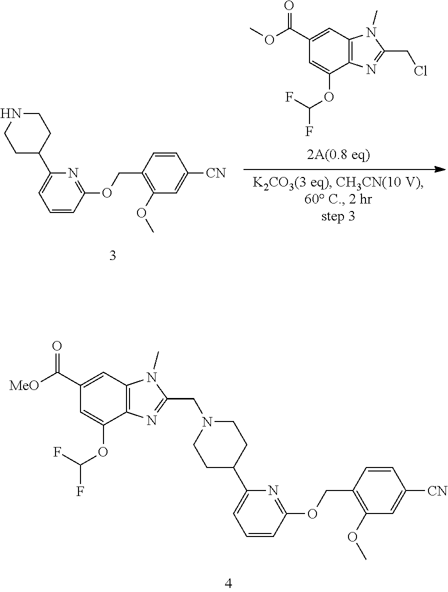 [SMILES: COc1cc(C#N)ccc1COc1cccc(C2CCNCC2)n1 ; COC(=O)c1cc(OC(F)F)c2nc(CCl)n(C)c2c1 ; "2A(0.8 eq)" ; "K2CO3(3 eq), CH3CN(10 V),60° C., 2 hrstep 3" ; COC(=O)c1cc(OC(F)F)c2nc(CN3CCC(c4cccc(OCc5ccc(C#N)cc5OC)n4)CC3)n(C)c2c1]

To a mixture of 3 (110 mg, 340.15 μmol, 1 eq) and 2A (82.91 mg, 272.12 μmol, 0.8 eq) in ACN (1.1 mL) was added K₂CO₃ (141.03 mg, 1.02 mmol, 3 eq). The mixture was stirred at 60° C. for 2 hours. LC-MS showed 2A was consumed completely and one main peak with desired mass was detected. The mixture filtered and concentrated in vacuum. The crude product was triturated with MeOH (1 mL), PE (10 mL) and EtOAc (10 mL) at 25° C. for 15 min, then filtered and the filter cake concentrated in vacuum. 4 (98 mg, 150.74 μmol, 44.32% yield) was obtained as a white solid.

LCMS: RT=0.472 min, MS cal.: 591.2, [M+H]⁺=592.3

¹H NMR (400 MHz, DMSO-d6) δ=8.11 (d, J=1.2 Hz, 1H) 7.83-7.46 (m, 1H) 7.60-7.66 (m, 1H) 7.52 (d, J=7.6 Hz, 3H) 7.40 (dd, J=8.0, 1.2 Hz, 1H) 6.86 (d, J=7.2 Hz, 1H) 6.71 (d, J=8.4 Hz, 1H) 5.37 (s, 2H) 3.97 (s, 3H) 3.89 (d, J=4.0 Hz, 6H) 3.85 (s, 2H) 2.86-2.99 (m, 2H) 2.56-2.62 (m, 1H) 2.21 (br t, J=10.4 Hz, 2H) 1.72-1.82 (m, 2H) 1.57-1.71 (m, 2H)

### General Procedure for Preparation of Compound 156 (5):

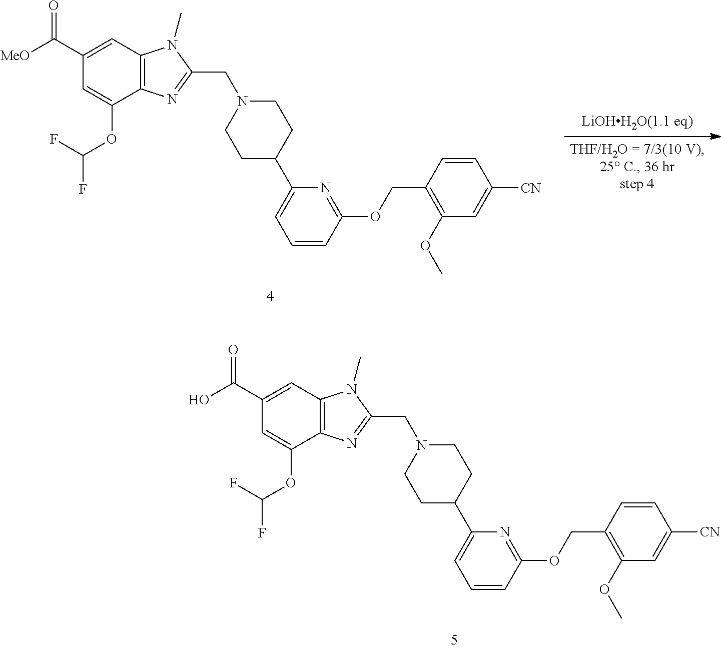

To a mixture of 4 (95 mg, 160.58 μmol, 1 eq) in THF (0.7 mL) and H₂O (0.3 mL) was added LiOH·H₂O (7.41 mg, 176.64 μmol, 1.1 eq). The mixture was stirred at 25° C. for 36 hours. LC-MS showed 4 was consumed completely and one main peak with desired mass was detected. The reaction was filtered directly. The residue was purified by prep-HPLC (column: Waters Xbridge Prep OBD C18 150*40 mm*10 um; mobile phase: [H₂O (10 mM NH₄HCO₃)-ACN]; gradient: 30%-60% B over 8.0 min). 2-((4-(6-((4-Cyano-2-methoxybenzyl)oxy)pyridin-2-yl)piperidin-1-yl)methyl)-4-(difluoromethoxy)-1-methyl-1H-benzo[d]imidazole-6-carboxylic acid (68 mg, 117.36 μmol, 73.08% yield) was obtained as a white solid.

LCMS: RT=2.679 min, MS cal.: 577.2, [M+H]⁺=578.2

HPLC: RT=10.790 min, purity: 99.68%

¹H NMR (400 MHz, DMSO-d₆) δ=8.07 (s, 1H) 7.81-7.44 (m, 1H) 7.59-7.67 (m, 1H) 7.47-7.55 (m, 3H) 7.40 (d, J=7.6 Hz, 1H) 6.86 (d, J=7.2 Hz, 1H) 6.71 (d, J=8.0 Hz, 1H) 5.37 (s, 2H) 3.95 (s, 3H) 3.89 (s, 3H) 3.84 (s, 2H) 2.92 (br d, J=10.8 Hz, 2H) 2.53-2.62 (m, 1H) 2.20 (br t, J=10.8 Hz, 2H) 1.72-1.81 (m, 2H) 1.58-1.71 (m, 2H)

### Example A201—Preparation of Compound 157. 2-((4-((6-(4-Cyano-2-fluorobenzyl)pyridin-2-yl)oxy)piperidin-1-yl)methyl)-4-(difluoromethoxy)-1-methyl-1H-benzo[d]imidazole-6-carboxylic acid (Compound 157) (6)

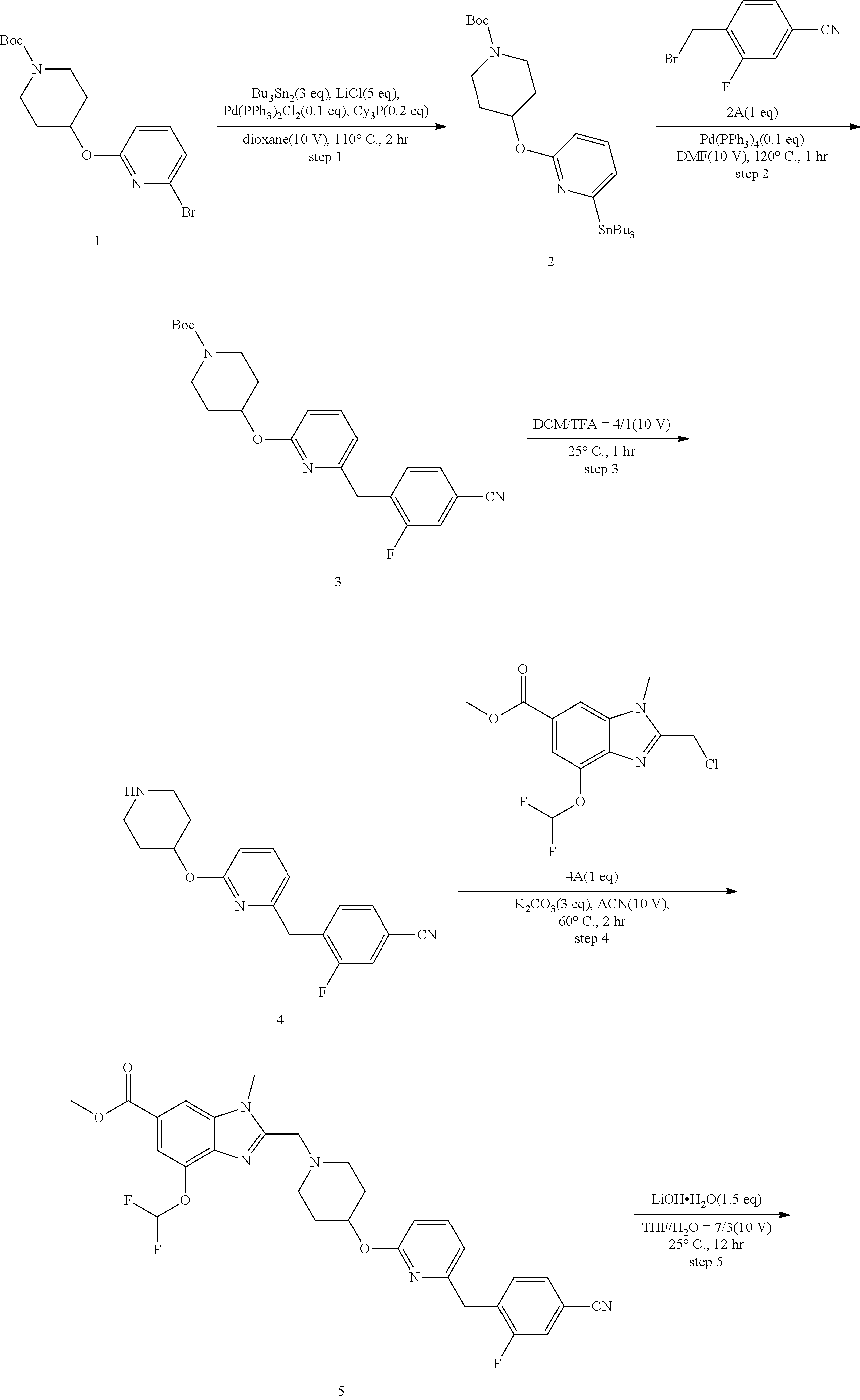

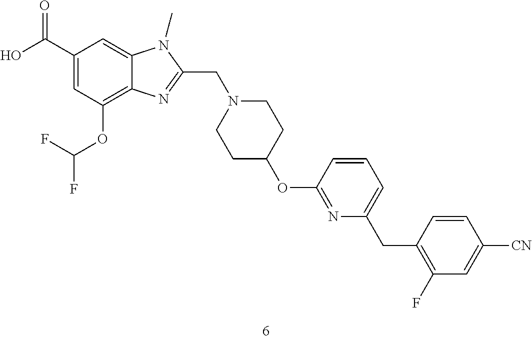 [SMILES: Cn1c(CN2CCC(Oc3cccc(Cc4ccc(C#N)cc4F)n3)CC2)nc2c(OC(F)F)cc(C(=O)O)cc21]

### General Procedure for Preparation of Intermediate 2:

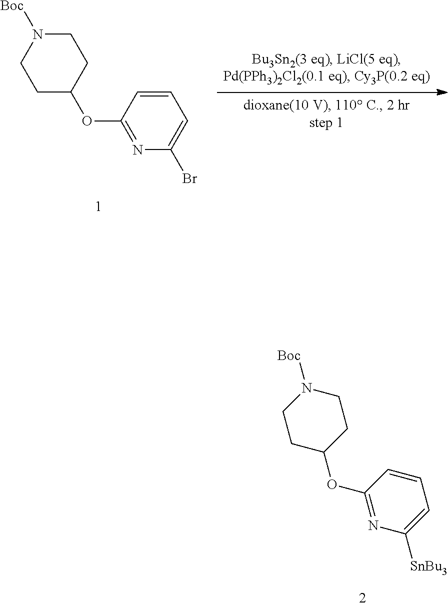

A mixture of 1 1.5 g, 4.20 mmol, 1 eq), LiCl (890.03 mg, 20.99 mmol, 430.38 μL, 5 eq), Pd(PPh₃)₂Cl₂ (294.71 mg, 419.88 μmol, 0.1 eq), Cy₃P (235.49 mg, 839.77 μmol, 272.25 μL, 0.2 eq) and Bu₃Sn₂ (7.31 g, 12.60 mmol, 6.31 mL, 3 eq) in dioxane (15 mL) was degassed and purged with N₂ for 3 times, and then the mixture was stirred at 110° C. for 2 hrs under N₂ atmosphere. TLC indicated 1 was consumed completely and two new spots formed. The reaction was clean according to TLC (Petroleum ether:Ethyl acetate=10:1, Product Rr-0.61). The residue mixture was diluted with H₂O (50 mL) and extracted with EtOAc (50 mL*2). The combined organic layers were washed with brine (50 mL), dried over Na₂SO₄, filtered, and concentrated under reduced pressure to give a residue. The residue was purified by column chromatography (SiO₂, Petroleum ether/Ethyl acetate=100/1 to 97/3, TLC-Petroleum ether/Ethyl acetate=10/1, Product R_{f}=0.61). 2 (1.9 g, 3.01 mmol, 71.58% yield, 89.75% yield) was obtained as a colorless oil.

LCMS: RT=2.147 min, MS cal.: 568.27, [M+H]⁺=569.1

¹H NMR (400 MHz, CHCl3-d) δ=7.43-7.36 (m, 1H), 7.01-6.93 (m, 1H), 6.53 (d, J=8.4 Hz, 1H), 5.28 (td, J=4.0, 7.9 Hz, 1H), 3.86-3.74 (m, 2H), 3.31-3.21 (m, 2H), 1.98 (br d, J=3.2 Hz, 2H), 1.78-1.69 (m, 2H), 1.61-1.55 (m, 6H), 1.48 (s, 9H), 1.37-1.30 (m, 7H), 1.10-1.04 (m, 5H), 0.89 (t, J=7.2 Hz, 9H)

### General Procedure for Preparation of Intermediate 3:

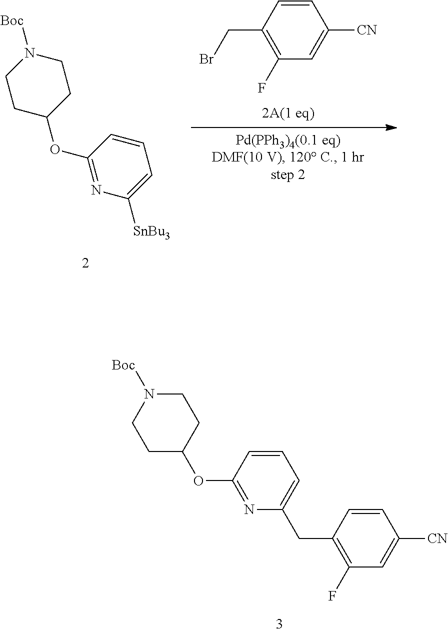

A mixture of 2 (1.9 g, 3.35 mmol, 1 eq), 2A (716.73 mg, 3.35 mmol, 1 eq), Pd(PPh₃)₄ (386.96 mg, 334.87 μmol, 0.1 eq) in DMF (20 mL) was degassed and purged with N₂ for 3 times, and then the mixture was stirred at 120° C. for 1 hr under N₂ atmosphere. TLC indicated 2 was consumed completely and one new spot formed. The reaction was clean according to TLC (Petroleum ether:Ethyl acetate=5:1, Product R_{f}=0.28). The residue mixture was diluted with H₂O (20 mL) and extracted with EtOAc (20 mL*3). The combined organic layers were washed with brine (20 mL), dried over Na₂SO₄, filtered, and concentrated under reduced pressure to give a residue. The residue was purified by column chromatography (SiO₂, Petroleum ether/Ethyl acetate=100/1 to 9/1, TLC-Petroleum ether/Ethyl acetate=5/1, Product R_{f}=0.26). The residue was further separated by prep-HPLC (neutral condition: column: Waters Xbridge Prep OBD C₁₈ 150*40 mm*10 um; mobile phase: [H₂O (10 mM NH₄HCO₃)-ACN]; gradient: 70%-95% B over 8.0 min). 3 (350 mg, 849.00 μmol, 25.35% yield, 99.81% purity) was obtained as a colorless oil.

LCMS: RT=1. 567 min, MS cal.: 411.20, [M+H]⁺=412.1

¹H NMR (400 MHz, CHLOROFORM-d) δ=7.50 (t, J=7.6 Hz, 1H), 7.42-7.37 (m, 2H), 7.35 (d, J=8.8 Hz, 1H), 6.73 (d, J=7.2 Hz, 1H), 6.56 (d, J=8.0 Hz, 1H), 5.06 (td, J=4.0, 7.7 Hz, 1H), 4.08 (s, 2H), 3.78-3.67 (m, 2H), 3.29-3.17 (m, 2H), 1.86 (br s, 2H), 1.65 (br d, J=9.2 Hz, 2H), 1.48 (s, 9H)

### General Procedure for Preparation of Intermediate 4:

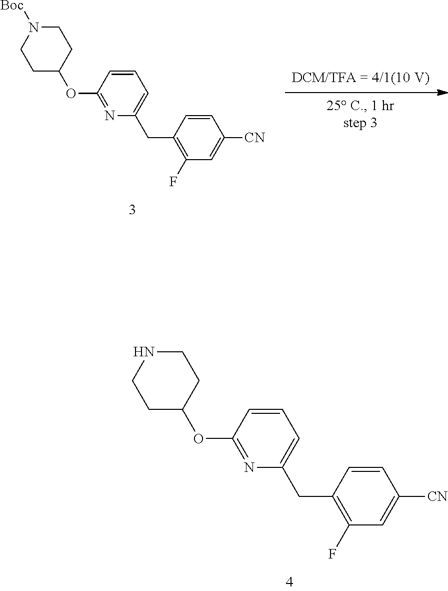

A mixture of 3 (350 mg, 850.61 μmol, 1 eq) in DCM (3.2 mL) and TFA (0.8 mL) was degassed and purged with N₂ for 3 times, and then the mixture was stirred at 25° C. for 1 hr under N₂ atmosphere. LC-MS showed 3 was consumed completely and one main peak with desired mass was detected. The reaction mixture was concentrated under reduced pressure to give a residue. 4 (360 mg, crude, TFA) was obtained as a colorless oil.

LCMS: RT=0.985 min, MS cal.: 311.14, [M+H]⁺=312.0

¹H NMR (400 MHz, CHLOROFORM-d) δ=8.09-7.96 (m, 1H), 7.84 (t, J=8.0 Hz, 1H), 7.46 (d, J=3.6 Hz, 2H), 7.39 (d, J=9.2 Hz, 1H), 6.95 (d, J=7.6 Hz, 1H), 6.88 (d, J=8.4 Hz, 1H), 5.24 (br s, 1H), 4.22 (s, 2H), 3.54-3.44 (m, 2H), 3.41-3.34 (m, 2H), 2.33-2.24 (m, 2H), 2.22-2.15 (m, 2H)

### General Procedure for Preparation of Intermediate 5:

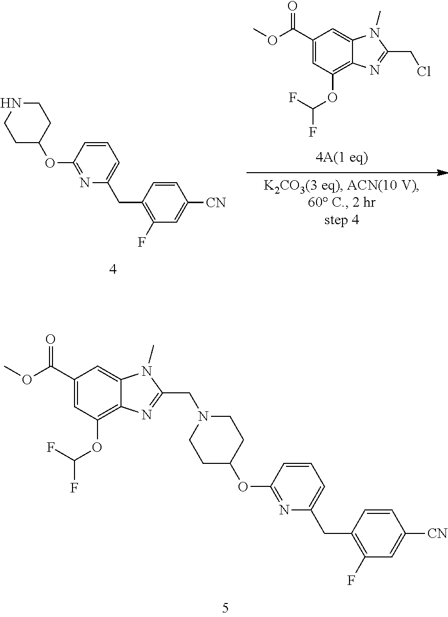 [SMILES: N#Cc1ccc(Cc2cccc(OC3CCNCC3)n2)c(F)c1 ; COC(=O)c1cc(OC(F)F)c2nc(CCl)n(C)c2c1 ; "4A(1 eq)" ; "K2CO3(3 eq), ACN(10 V),60° C., 2 hrstep 4" ; COC(=O)c1cc(OC(F)F)c2nc(CN3CCC(Oc4cccc(Cc5ccc(C#N)cc5F)n4)CC3)n(C)c2c1]

A mixture of 4 (360 mg, 846.31 μmol, 1 eq, TFA), 4A (257.85 mg, 846.31 μmol, 1 eq), K₂CO₃ (350.89 mg, 2.54 mmol, 3 eq) in ACN (4 mL) was degassed and purged with N₂ for 3 times, and then the mixture was stirred at 60° C. for 2 hrs under N₂ atmosphere. LC-MS showed 4 was consumed completely and one main peak with desired mass was detected. The residue mixture was diluted with H₂O (30 mL) and extracted with EtOAc (30 mL*3). The combined organic layers were washed with brine (30 mL), dried over Na₂SO₄, filtered and concentrated under reduced pressure to give a residue. The residue was purified by column chromatography (SiO₂, Petroleum ether/Ethyl acetate=100/1 to 1/1, TLC-Petroleum ether/Ethyl acetate=2/1, Product R_{f}=0.36). 5 (400 mg, 660.22 μmol, 78.01% yield, 95.66% purity) was obtained as a white solid.

LCMS: RT=0.461 min, MS cal.: 579.21, [M+H]⁺=580.3

¹H NMR (400 MHz, CHLOROFORM-d) δ=8.00-7.97 (m, 1H), 7.72 (s, 1H), 7.48 (d, J=7.8 Hz, 1H), 7.40-7.37 (m, 2H), 7.34 (d, J=9.3 Hz, 1H), 7.32 (t, J=74.4 Hz, 1H), 6.73 (d, J=7.2 Hz, 1H), 6.55 (d, J=8.2 Hz, 1H), 4.94 (br d, J=2.3 Hz, 1H), 4.07 (s, 2H), 4.00-3.95 (m, 6H), 3.88 (br d, J=6.5 Hz, 2H), 2.79 (br d, J=8.2 Hz, 2H), 2.40 (br s, 2H), 1.92 (br d, J=0.7 Hz, 2H), 1.72 (br d, J=3.4 Hz, 2H)

### General Procedure for Preparation of Compound 157 (6):

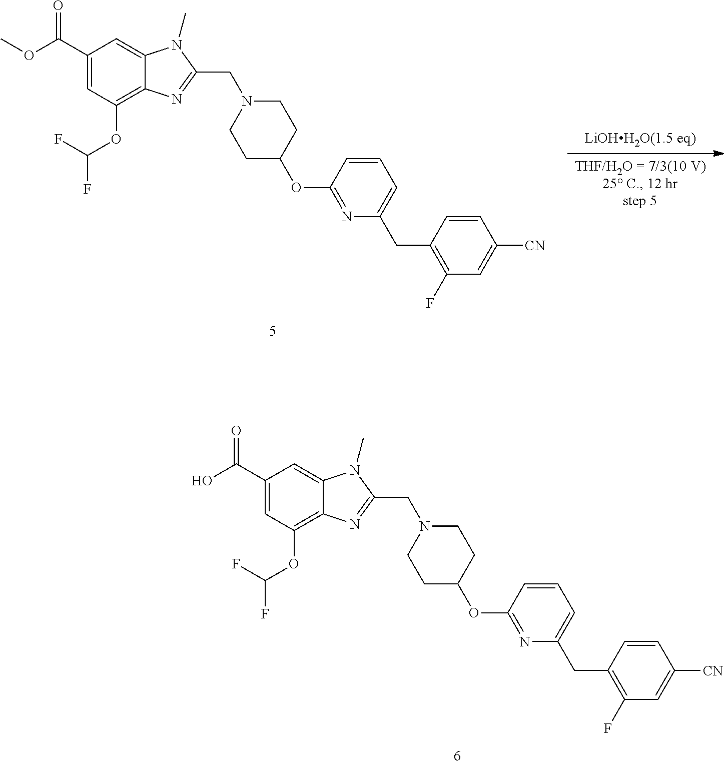

A mixture of 5 (100 mg, 172.54 μmol, 1 eq), LiOH·H₂O (10.86 mg, 258.81 μmol, 1.5 eq) in THF (0.7 mL) and H₂O (0.3 mL) was degassed and purged with N₂ for 3 times, and then the mixture was stirred at 25° C. for 12 hrs under N₂ atmosphere. LC-MS showed 5 was consumed completely and one main peak with desired mass was detected. The reaction mixture was diluted with ACN 0.3 mL. The liquid was purified by prep-HPLC (column: Waters Xbridge Prep OBD C₁₈ 150*40 mm*10 um; mobile phase: [H₂O (10 mM NH₄HCO₃)-ACN]; gradient: 25%-55% B over 8.0 min). 2-((4-((6-(4-Cyano-2-fluorobenzyl)pyridin-2-yl)oxy)piperidin-1-yl)methyl)-4-(difluoromethoxy)-1-methyl-1H-benzo[d]imidazole-6-carboxylic acid (28.39 mg, 49.12 μmol, 28.47% yield, 97.84% purity) was obtained as a white solid.

LCMS: RT=0.652 min, MS cal.: 565.19, [M+H]⁺=566.2

¹H NMR (400 MHz, METHANOL-d₄) δ 8.09 (s, 1H), 7.67 (s, 1H), 7.58-7.47 (m, 4H), 7.23 (t, J=74.4 Hz, 1H), 6.82 (d, J=7.2 Hz, 1H), 6.55 (d, J=8.0 Hz, 1H), 4.95 (br s, 1H), 4.11 (s, 2H), 3.99 (s, 3H), 3.93 (s, 2H), 2.87-2.79 (m, 2H), 2.43 (br t, J=9.2 Hz, 2H), 1.92 (br d, J=11.2 Hz, 2H), 1.76-1.65 (m, 2H)

### Example A202—Preparation of Compound 158. 2-((4-(3-((4-Cyano-2-fluorobenzyl)oxy)pyridin-2-yl)piperidin-1-yl)methyl)-4-(difluoromethoxy)-1-methyl-1H-benzo[d]imidazole-6-carboxylic acid (Compound 158) (7)

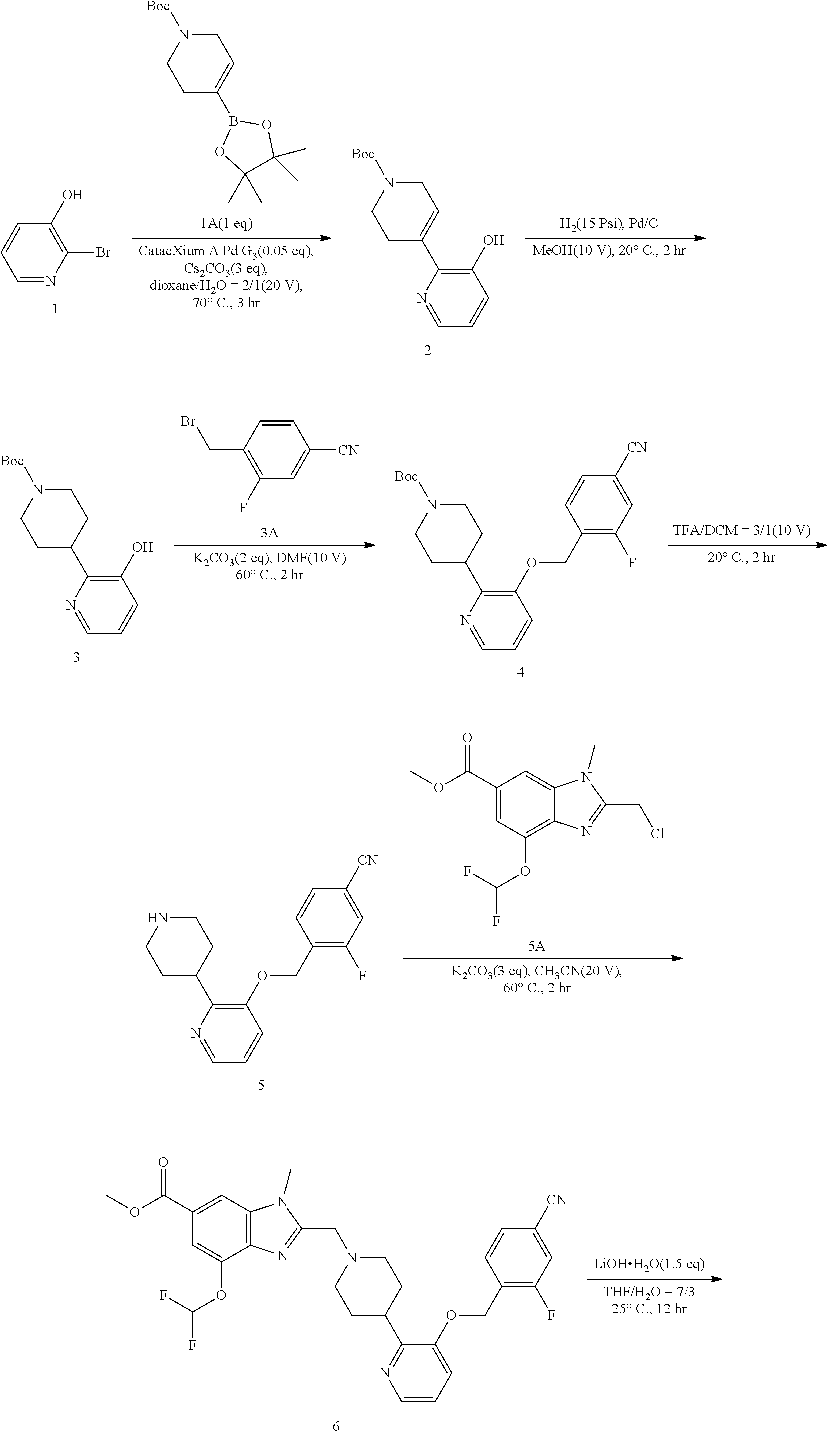

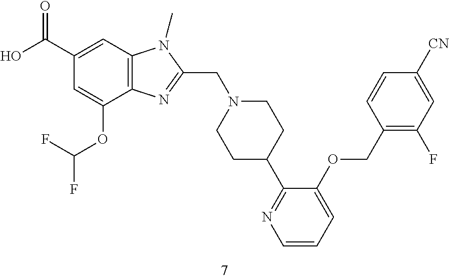 [SMILES: Cn1c(CN2CCC(c3ncccc3OCc3ccc(C#N)cc3F)CC2)nc2c(OC(F)F)cc(C(=O)O)cc21]

### General Procedure for Preparation of Intermediate 2:

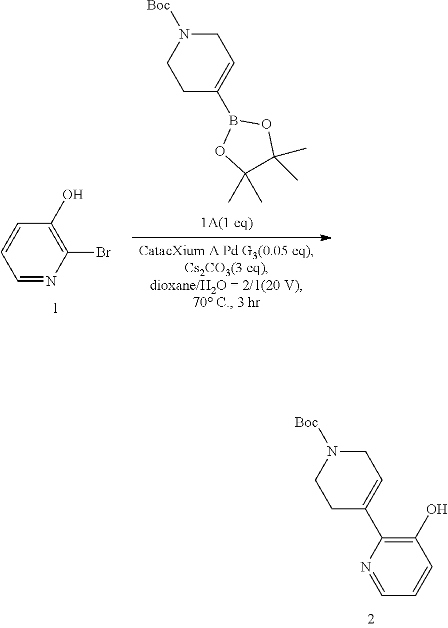

A mixture of 1 (200 mg, 1.15 mmol, 1 eq), 1A (390.96 mg, 1.26 mmol, 1.1 eq), Pd(dppf)Cl₂ (42.05 mg, 57.47 μmol, 0.05 eq), Cs₂CO₃ (561.77 mg, 1.72 mmol, 1.5 eq) in dioxane (4 mL) and H₂O (2 mL) was degassed and purged with N₂ for 3 times, and then the mixture was stirred at 70° C. for 3 hr under N₂ atmosphere. LC-MS showed 1 was consumed completely and desired mass was detected. The suspension was filtered through a pad of Celite, and filter cake was washed with EtOAc (5 mL*3). The combined filtrates were diluted with H₂O 15 mL and extracted with EtOAc 45 mL (15 mL*3). The combined organic layers were washed with brine 15 mL, dried over Na₂SO₄, filtered, and concentrated under reduced pressure to give a residue. The residue was purified by column chromatography (SiO₂, Petroleum ether/Ethyl acetate=10/1 to 1/1) (Rf=0.42). 2 (180 mg, 651.39 μmol, 56.67% yield) was obtained as colorless oil.

LCMS: RT=1.515 min, MS cal.: 276.3, [M+H]⁺=277.1

### General Procedure for Preparation of Intermediate 3:

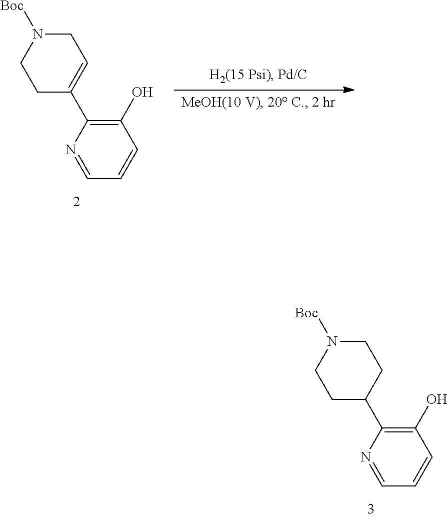 [SMILES: CC(C)(C)OC(=O)N1CC=C(c2ncccc2O)CC1 ; "H2(15 Psi), Pd/C" ; "MeOH(10 V), 20° C., 2 hr" ; CC(C)(C)OC(=O)N1CCC(c2ncccc2O)CC1]

MeOH (2 mL) was charged to the round bottom flask, then 2 (180 mg, 651.39 μmol, 1 eq) was added to the mixture at 20° C. Pd/C (80 mg, 10% purity) was added to the mixture under N₂ atmosphere. The suspension was degassed and purged with H₂ for 3 times. The mixture was stirred under H₂ (15 Psi) at 20° C. for 2 hr. The reaction was monitored by LCMS. The mixture was filtered through a Celite pad and washed by MeOH (5 mL*3), and the filtrate was concentrated to give the crude product. 3 (150 mg, 538.90 μmol, 82.73% yield) was obtained as colorless oil.

LCMS: RT=1.529 min, MS cal.: 278.3, [M+H]⁺=279.2

¹H NMR (400 MHz, CHCl3-d) δ=8.07 (dd, J=1.2, 3.6 Hz, 1H), 7.12-7.05 (m, 1H), 6.99 (dd, J=4.8, 8.0 Hz, 1H), 4.33-4.00 (m, 2H), 3.22-3.06 (m, 1H), 2.85-2.72 (m, 2H), 1.78-1.65 (m, 4H), 1.40 (s, 9H)

### General Procedure for Preparation of Intermediate 4:

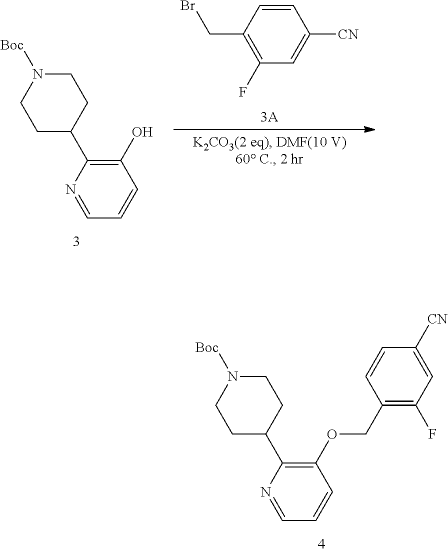

To a solution of 3 (150 mg, 538.90 μmol, 1 eq) in DMF (2 mL) was added K₂CO₃ (148.96 mg, 1.08 mmol, 2 eq) and 3A (115.34 mg, 538.90 μmol, 1 eq). The mixture was stirred at 60° C. for 2 hr. LC-MS showed 3 was consumed completely and desired mass was detected. The reaction mixture was diluted with H₂O 10 mL and extracted with EtOAc 30 mL (10 mL*3). The combined organic layers were washed with brine 15 mL, dried over Na₂SO₄, filtered, and concentrated under reduced pressure to give a residue. The residue was purified by column chromatography (SiO₂, Petroleum ether/Ethyl acetate=10/1 to 1/1) (Rf=0.52). 4 (130 mg, 315.94 μmol, 58.63% yield) was obtained as a white solid.

LCMS: RT=0.848 min, MS cal.: 411.4, [M+H]⁺=412.2

¹H NMR (400 MHz, CHCl3-d) δ=8.24 (br d, J=4.0 Hz, 1H), 7.66-7.60 (m, 1H), 7.58-7.52 (m, 1H), 7.45 (d, J=8.8 Hz, 1H), 7.23-7.12 (m, 2H), 5.22 (s, 2H), 4.36-4.19 (m, 2H), 3.39-3.27 (m, 1H), 2.94-2.77 (m, 2H), 1.96-1.75 (m, 4H), 1.48 (s, 9H)

### General Procedure for Preparation of Intermediate 5:

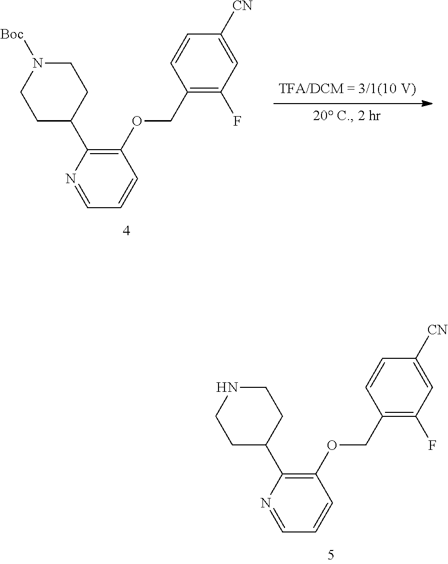

To a solution of 4 (110 mg, 267.34 μmol, 1 eq) in DCM (1 mL) was added TFA (460.50 mg, 4.04 mmol, 300.00 μL, 15.11 eq). The mixture was stirred at 20° C. for 2 hr. TLC (Petroleum ether:Ethyl acetate=2:1, product Rf=0.17) indicated 4 was consumed completely and one new spot formed. The reaction was clean according to TLC. The reaction mixture was concentrated under reduced pressure to remove solvent. 5 (113 mg, 265.65 μmol, 99.37% yield, TFA) was obtained as colorless oil.

LCMS: RT=0.917 min, MS cal.: 311.3, [M+H]⁺=312.1

### General Procedure for Preparation of Intermediate 6:

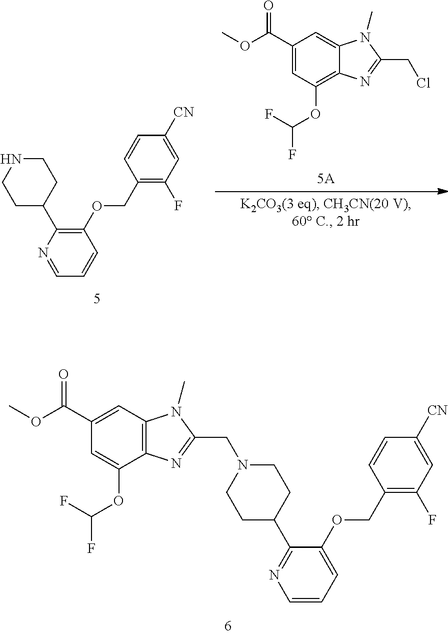 [SMILES: N#Cc1ccc(COc2cccnc2C2CCNCC2)c(F)c1 ; COC(=O)c1cc(OC(F)F)c2nc(CCl)n(C)c2c1 ; "5A" ; "K2CO3(3 eq), CH3CN(20 V),60° C., 2 hr" ; COC(=O)c1cc(OC(F)F)c2nc(CN3CCC(c4ncccc4OCc4ccc(C#N)cc4F)CC3)n(C)c2c1]

To a solution of 5 (113 mg, 265.65 μmol, 1 eq, TFA) in ACN (2 mL) was added K₂CO₃ (110.14 mg, 796.94 μmol, 3 eq) and 5A (80.94 mg, 265.65 μmol, 1 eq) at 25° C. The mixture was stirred at 60° C. for 2 hr. LC-MS showed 5 was consumed completely and desired mass was detected. The residue was diluted with H₂O 15 mL and extracted with EtOAc 45 mL (15 mL*3). The combined organic layers were washed with brine 15 mL, dried over Na₂SO₄, filtered, and concentrated under reduced pressure to give a residue. The residue was purified by column chromatography (SiO₂, Petroleum ether:Ethyl acetate=10/1 to 5/1) (Rf=0.45). 6 (120 mg, 207.05 μmol, 77.94% yield) was obtained as a yellow solid.

LCMS: RT=2.146 min, MS cal.: 579.5, [M+H]⁺=580.2

### General Procedure for Preparation of Compound 158 (7):

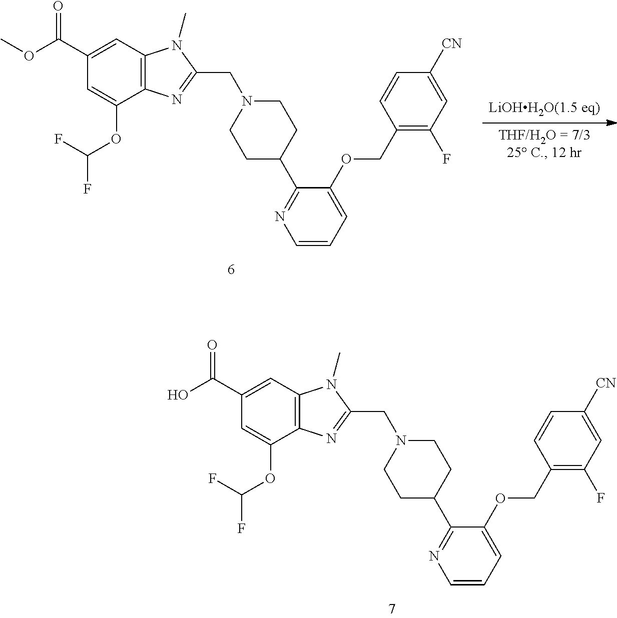

To a solution of 6 (110 mg, 189.80 μmol, 1 eq) in THF (0.7 mL) was added LiOH·H₂O (11.95 mg, 284.69 μmol, 1.5 eq) in H₂O (0.3 mL). The mixture was stirred at 25° C. for 12 hr. LC-MS showed 6 was consumed completely and desired mass was detected. The reaction was added citric acid (0.5 M) to pH=8, then the mixture was purified directly. The residue was purified by prep-HPLC (neutral condition; column: Waters Xbridge BEH C18 100*30 mm*10 um; mobile phase: [H₂O (10 mM NH₄HCO₃)-ACN]; gradient: 15%-45% B over 8.0 min). 2-((4-(3-((4-Cyano-2-fluorobenzyl)oxy)pyridin-2-yl)piperidin-1-yl)methyl)-4-(difluoromethoxy)-1-methyl-1H-benzo[d]imidazole-6-carboxylic acid (25.29 mg, 44.66 μmol, 23.53% yield, 99.87% purity) was obtained as a white solid.

LCMS: RT=2.113 min, MS cal.: 565.5, [M+H]⁺=566.2

HPLC: RT=8.760 min, purity: 99.87%

¹H NMR (400 MHz, MeOH-d4) δ=8.13-8.06 (m, 2H), 7.78-7.71 (m, 1H), 7.69-7.63 (m, 3H), 7.48 (d, J=8.4 Hz, 1H), 7.27-7.20 (m, 1H), 7.24 (t, J=74 Hz, 1H), 5.30 (s, 2H), 4.01 (s, 3H), 4.00 (s, 2H), 3.28-3.23 (m, 1H), 3.10 (br d, J=11.4 Hz, 2H), 2.42 (br t, J=11.2 Hz, 2H), 1.99 (dq, J=3.2, 12.4 Hz, 2H), 1.81 (br d, J=12.0 Hz)

### Example A203—Preparation of Compound 159. 2-((4-(3-((4-Chloro-2-fluorobenzyl)oxy)phenyl)-3,6-dihydropyridin-1(2H)-yl)methyl)-4-(difluoromethoxy)-1-methyl-1H-benzo[d]imidazole-6-carboxylic acid (Compound 159) (5)

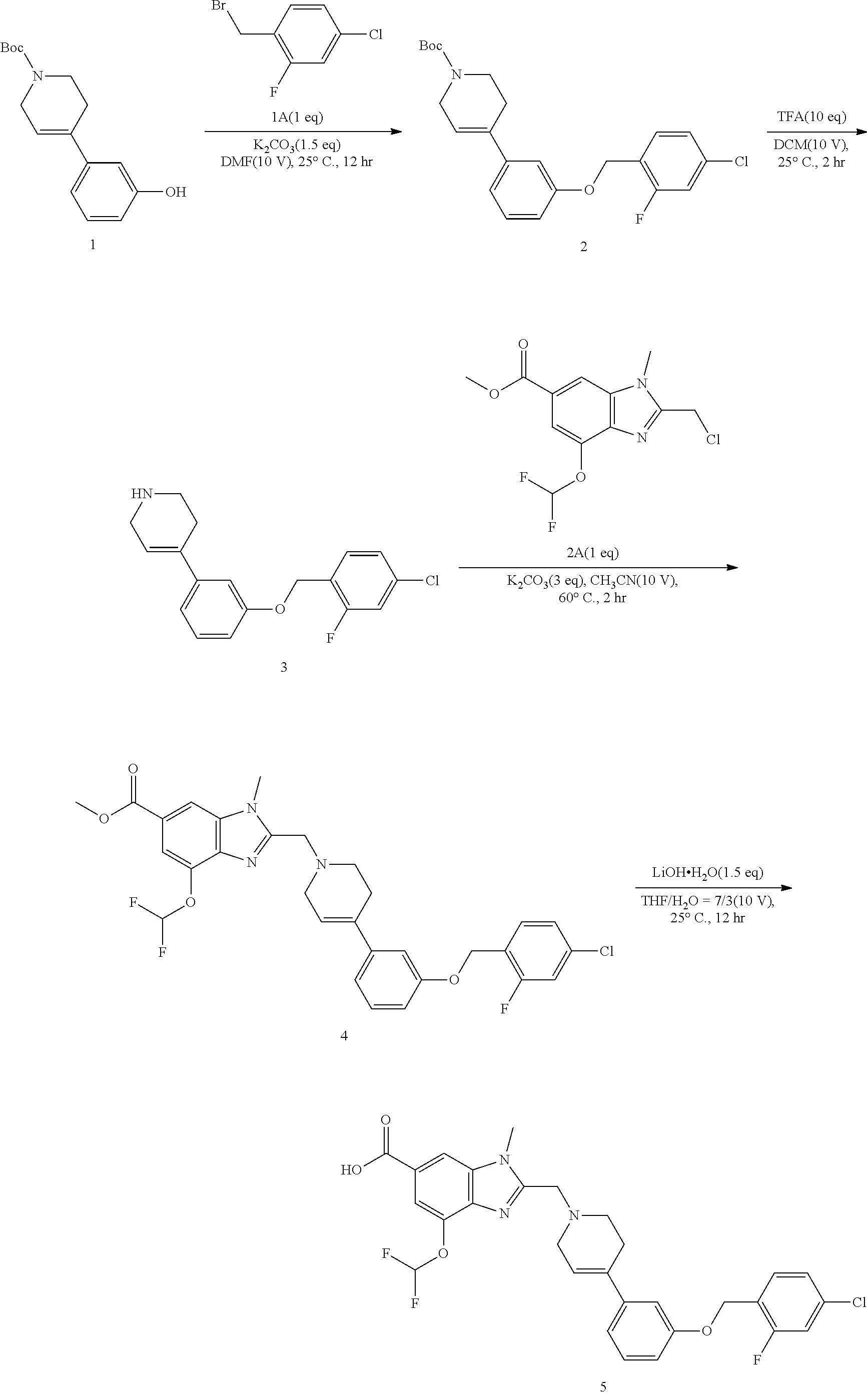

### General Procedure for Preparation of Intermediate 2:

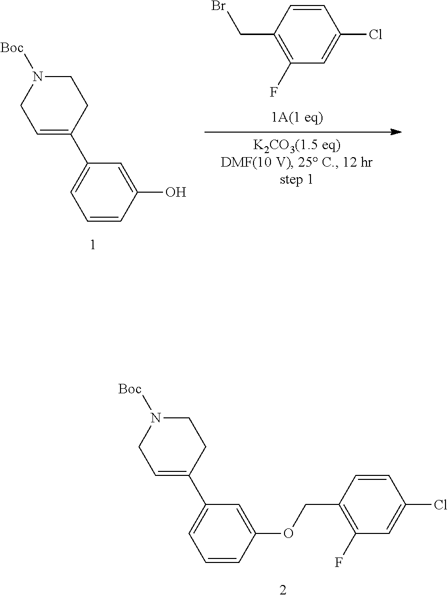

To a solution of 1 (220 mg, 799.01 μmol, 1 eq), 1A (178.55 mg, 799.01 μmol, 1 eq) in DMF (2.4 mL) was added K₂CO₃ (165.65 mg, 1.20 mmol, 1.5 eq). The mixture was stirred at 25° C. for 12 hr. LCMS indicated 1 was consumed completely and one main peak with desired mass was detected. The residue was poured into water (10 mL). The aqueous phase was extracted with EtOAc (10 mL*3). The combined organic phase was washed with brine (10 mL*2), dried with anhydrous Na₂SO₄, filtered and concentrated in vacuum. The residue was purified by column chromatography (SiO₂, Petroleum ether/EtOAc=1/0 to 10/1). 2 (237.5 mg, 568.32 μmol, 71.13% yield) was a white solid.

LCMS: RT=2.443 min, MS cal.: 417.15, [M-Boc]⁺=318.2

¹H NMR (400 MHz, DMSO-d6) δ=7.60 (t, J=8.0 Hz, 1H), 7.50 (dd, J=2.0, 10.0 Hz, 1H), 7.34 (dd, J=1.6, 8.4 Hz, 1H), 7.27 (t, J=8.0 Hz, 1H), 7.12-6.99 (m, 2H), 6.96-6.88 (m, 1H), 6.17 (br s, 1H), 5.15 (s, 2H), 3.99 (br s, 2H), 3.60-3.48 (m, 2H), 2.48-2.41 (m, 2H), 1.42 (s, 9H)

### General Procedure for Preparation of Intermediate 3:

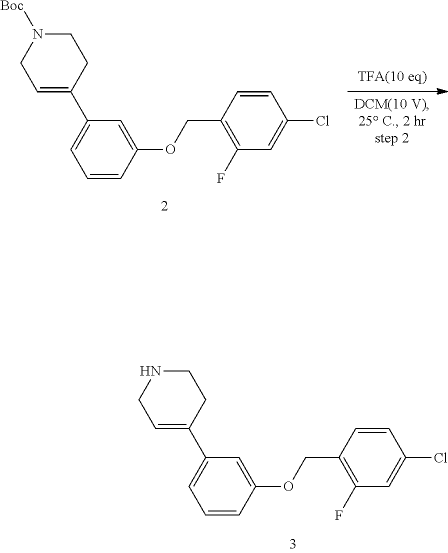 [SMILES: CC(C)(C)OC(=O)N1CC=C(c2cccc(OCc3ccc(Cl)cc3F)c2)CC1 ; "DCM(10 V),25° C., 2 hrstep 2" ; "TFA(10 eq)" ; Fc1cc(Cl)ccc1COc1cccc(C2=CCNCC2)c1]

To a solution of 2 (200 mg, 478.58 μmol, 1 eq) in DCM (0.3 mL) was added TFA (545.68 mg, 4.79 mmol, 355.49 μL, 10 eq). The mixture was stirred at 25° C. for 2 hr. LCMS indicated 2 was consumed completely and one main peak with desired mass was detected. The reaction mixture was concentrated under reduced pressure to remove solvent. 3 (206 mg, 477.06 μmol, 99.68% yield, TFA) was a white solid.

LCMS: RT=1.339 min, MS cal.: 317.1, [M+H]⁺=318.2

¹H NMR (400 MHz, DMSO-d6) δ=8.89 (br s, 2H), 7.60 (dt, J=2.0, 8.0 Hz, 1H), 7.50 (d, J=9.6 Hz, 1H), 7.40-7.25 (m, 2H), 7.14-7.04 (m, 2H), 7.00 (br d, J=8.8 Hz, 1H), 6.23 (br s, 1H), 5.16 (s, 2H), 3.76 (br s, 2H), 3.32 (br d, J=2.4 Hz, 2H), 2.66 (br s, 2H).

### General Procedure for Preparation of Intermediate 4:

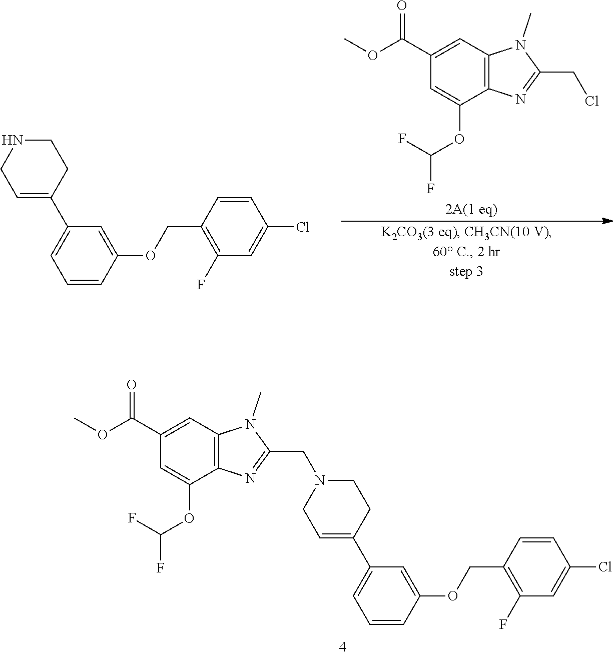 [SMILES: Fc1cc(Cl)ccc1COc1cccc(C2=CCNCC2)c1 ; COC(=O)c1cc(OC(F)F)c2nc(CCl)n(C)c2c1 ; COC(=O)c1cc(OC(F)F)c2nc(CN3CC=C(c4cccc(OCc5ccc(Cl)cc5F)c4)CC3)n(C)c2c1]

To a solution of 3 (120 mg, 393.86 μmol, 1 eq) and 2A (170.07 mg, 393.86 μmol, 1 eq, TFA) in CH₃CN (2 mL) was added K₂CO₃ (163.31 mg, 1.18 mmol, 3 eq). The mixture was stirred at 60° C. for 2 hr. LCMS indicated 3 was consumed completely and one main peak with desired mass was detected. The reaction mixture was poured into water (10 mL), extracted with EtOAc (10 mL*3). The combined organic phase was washed with brine (10 mL*2), dried with anhydrous Na₂SO₄, filtered and concentrated in vacuum. The residue was purified by column chromatography (SiO₂, Petroleum ether/EtOAc=1/0 to 3/1). 4 (200 mg, 341.30 μmol, 86.65% yield) was a white solid.

LCMS: RT=2.614 min, MS cal.: 585.16, [M+H]⁺=586.1

¹H NMR (400 MHz, DMSO-d₆) δ=8.10 (s, 1H), 7.65 (t, J=74.4 Hz, 1H), 7.58 (t, J=8.4 Hz, 1H), 7.54 (s, 1H), 7.51-7.46 (m, 1H), 7.33 (d, J=8.4 Hz, 1H), 7.25 (t, J=8.0 Hz, 1H), 7.06-6.99 (m, 2H), 6.90 (d, J=8.4 Hz, 1H), 6.18 (s, 1H), 5.13 (s, 2H), 3.98-3.92 (m, 4H), 3.90 (s, 3H), 3.20 (s, 2H), 2.75 (t, J=5.4 Hz, 2H), 2.54 (s, 1H), 2.48-2.44 (m, 2H)

### General Procedure for Preparation of Compound 159 (5):

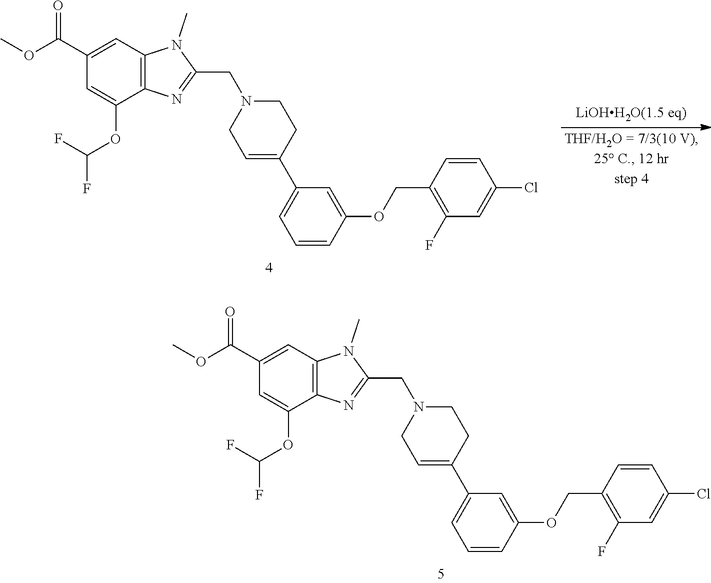 [SMILES: COC(=O)c1cc(OC(F)F)c2nc(CN3CC=C(c4cccc(OCc5ccc(Cl)cc5F)c4)CC3)n(C)c2c1 ; COC(=O)c1cc(OC(F)F)c2nc(CN3CC=C(c4cccc(OCc5ccc(Cl)cc5F)c4)CC3)n(C)c2c1]

To a solution of 4 (90 mg, 153.58 μmol, 1 eq) in THF (0.7 mL) and H₂O (0.3 mL) was added LiOH. H₂O (9.67 mg, 230.38 μmol, 1.5 eq). The mixture was stirred at 25° C. for 12 hr. LCMS indicated 4 was consumed completely and one main peak with desired mass was detected. The residue was purified by prep-HPLC (column: Phenomenex Gemini-NX 80*30 mm*3 um; mobile phase: [H₂O (10 mM NH₄HCO₃)-ACN]; gradient: 30%-60% B over 8.0 min). 2-((4-(3-((4-Chloro-2-fluorobenzyl)oxy)phenyl)-3,6-dihydropyridin-1(2H)-yl)methyl)-4-(difluoromethoxy)-1-methyl-1H-benzo[d]imidazole-6-carboxylic acid (60 mg, 102.59 μmol, 66.80% yield, 97.8% purity) was a white solid

LCMS: RT=2.848 min, MS cal.: 660.24, [M+H]⁺=661.3

HPLC: RT=7.479 min

¹H NMR (400 MHz, DMSO-d6) δ=8.06 (s, 1H), 7.64 (t, J=74.4 Hz, 1H), 7.59 (t, J=8.4 Hz, 1H), 7.54-7.47 (m, 2H), 7.33 (dd, J=2.0, 8.4 Hz, 1H), 7.25 (t, J=8.0 Hz, 1H), 7.06-7.01 (m, 2H), 6.90 (d, J=8.4 Hz, 1H), 6.18 (s, 1H), 5.13 (s, 2H), 3.96 (s, 2H), 3.94 (s, 3H), 3.23-3.18 (m, 2H), 2.75 (t, J=5.6 Hz, 2H), 2.48-2.43 (m, 2H)

### Example A204—Preparation of Compound 160. 2-((4-(6-((4-Cyano-2-fluorobenzyl)oxy)pyridin-2-yl)piperidin-1-yl)methyl)-4-((1-fluoro-2-methylpropan-2-yl)oxy)-1-methyl-1H-benzo[d]imidazole-6-carboxylic acid (Compound 160) (10)

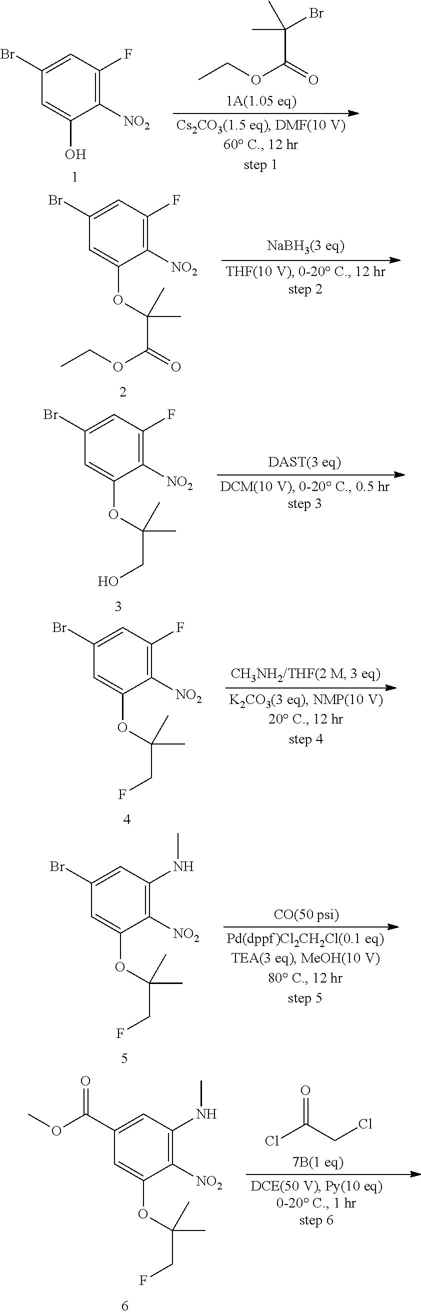

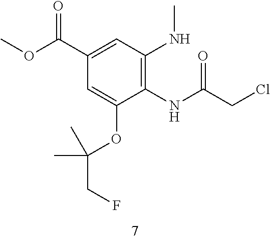 [SMILES: CNc1cc(C(=O)OC)cc(OC(C)(C)CF)c1NC(=O)CCl]

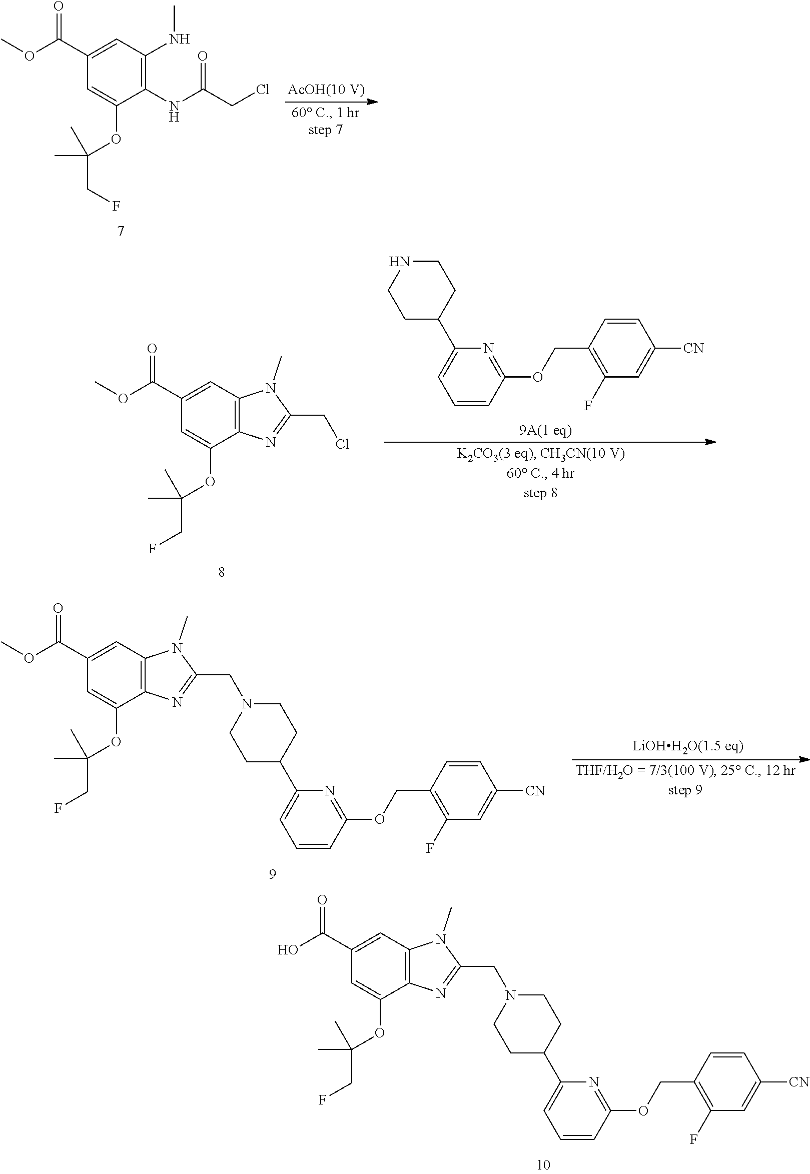 [SMILES: CNc1cc(C(=O)OC)cc(OC(C)(C)CF)c1NC(=O)CCl ; COC(=O)c1cc(OC(C)(C)CF)c2nc(CCl)n(C)c2c1 ; N#Cc1ccc(COc2cccc(C3CCNCC3)n2)c(F)c1 ; COC(=O)c1cc(OC(C)(C)CF)c2nc(CN3CCC(c4cccc(OCc5ccc(C#N)cc5F)n4)CC3)n(C)c2c1 ; Cn1c(CN2CCC(c3cccc(OCc4ccc(C#N)cc4F)n3)CC2)nc2c(OC(C)(C)CF)cc(C(=O)O)cc21]

### General Procedure for Preparation of Intermediate 2:

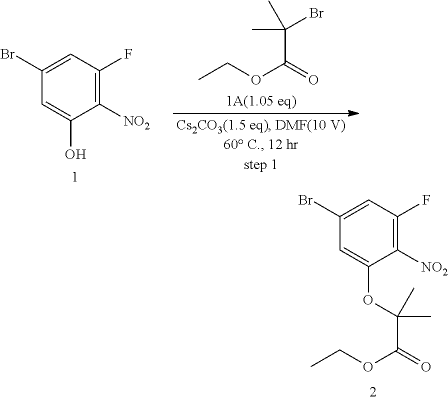

To a solution of 1 (2.60 g, 13.35 mmol, 1.96 mL, 1.05 eq) and 1A (3 g, 12.71 mmol, 1 eq) in DMF (26 mL) was added Cs₂CO₃ (6.21 g, 19.07 mmol, 1.5 eq). The mixture was stirred at 60° C. for 12 hr. LC-MS showed 1 was consumed completely and one main peak with desired m/z was detected. TLC (Petroleum ether:Ethyl acetate=3:1) indicated 1 was consumed completely and one new spot formed. The reaction was clean according to TLC. The reaction mixture was partitioned between Ethyl acetate 60 mL and H₂O 30 mL. The organic phase was separated, washed with brine 30 mL (15 mL*2), dried over Na₂SO₄, filtered, and concentrated under reduced pressure to give a residue. The residue was purified by column chromatography (SiO₂, Petroleum ether:Ethyl acetate=100/1 to 85/15). 2 (2 g, 5.71 mmol, 44.93% yield) was obtained as a green oil. 1H NMR (400 MHz, CHCl3-d) δ=7.09 (dd, J=1.6, 8.4 Hz, 1H), 6.92 (t, J=2.0 Hz, 1H), 4.32-4.26 (m, 2H), 1.64 (s, 6H), 1.29 (t, J=7.2 Hz, 4H)

### General Procedure for Preparation of Intermediate 3:

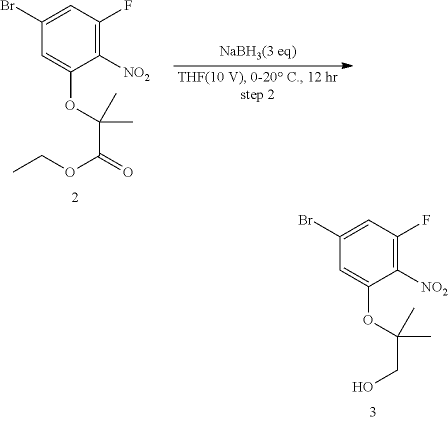 [SMILES: CCOC(=O)C(C)(C)Oc1cc(Br)cc(F)c1[N+](=O)[O-] ; CC(C)(CO)Oc1cc(Br)cc(F)c1[N+](=O)[O-]]

THF (20 mL) was charged to the 100 mL three-necked round bottom flask, then Compound 2 (2 g, 5.71 mmol, 1 eq) was added at 20° C. At 0° C., NaBH₄ (648.30 mg, 17.14 mmol, 3 eq) was added to the reaction mixture at 0° C. After the addition, the mixture was stirred at 20° C. for 12 hr. TLC (Petroleum ether:Ethyl acetate=3:1) indicated 2 was consumed completely and one new spot formed. The reaction was clean according to TLC. The mixture reaction was quenched by the addition of a saturated aq. NH₄CL (5 ml). The mixture was extracted by Ethyl acetate (30 mL*3). Then organic phase was washed by H₂O (20 mL*1) aq. brine (10 mL*1). The organic was dried (Na₂SO₄), filtered. The organic phase was concentrated under reduced pressure at 45° C. to give a residue. The residue was purified by column chromatography (SiO₂, Petroleum ether:Ethyl acetate=100/0 to 90/10). 3 (1.3 g, 4.22 mmol, 73.87% yield) was obtained as a green oil.

¹H NMR (400 MHz, DMSO-d₆) δ=7.07 (dd, J=1.6, 8.8 Hz, 1H), 7.02 (t, J=1.6 Hz, 1H), 3.93 (s, 2H), 1.33 (s, 6H)

### General Procedure for Preparation of Intermediate 4:

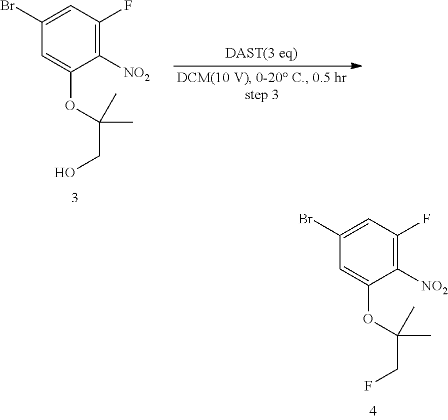 [SMILES: CC(C)(CO)Oc1cc(Br)cc(F)c1[N+](=O)[O-] ; CC(C)(CF)Oc1cc(Br)cc(F)c1[N+](=O)[O-]]

DCM (6 mL) was charged to the 25 mL three-necked round bottom flask, then 3 (500 mg, 1.62 mmol, 1 eq) was added at 20° C. At 0° C., DAST (784.75 mg, 4.87 mmol, 643.24 μL, 3 eq) was added to the reaction mixture at 0° C. After the addition, the mixture was stirred at 20° C. for 0.5 hr. TLC (Petroleum ether:Ethyl acetate=5:1) indicated 3 was consumed completely and one new spot formed. The reaction was clean according to TLC. The mixture reaction was quenched by the addition of a saturated aq. NaHCO₃ (2 ml). The mixture was extracted by Ethyl acetate (30 mL*3). Then organic phase was washed by H₂O (20 mL*1) aq. brine (10 mL*1). The organic was dried (Na₂SO₄), filtered. The organic phase was concentrated under reduced pressure at 45° C. to give a crude product. 4 (478 mg, crude) was obtained as a green oil, the crude product was used to next step without further purification.

¹H NMR (400 MHz, DMSO-d₆) δ=7.10-7.06 (m, 1H), 7.03 (t, J=1.6 Hz, 1H), 4.08 (s, 1H), 4.04 (s, 1H), 1.50 (s, 3H), 1.45 (s, 3H)

### General Procedure for Preparation of Intermediate 5:

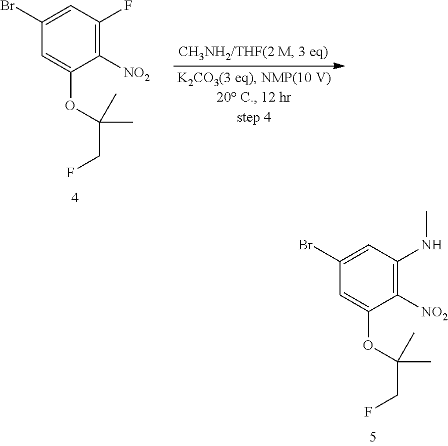 [SMILES: CC(C)(CF)Oc1cc(Br)cc(F)c1[N+](=O)[O-] ; CNc1cc(Br)cc(OC(C)(C)CF)c1[N+](=O)[O-]]

NMP (7.7 mL) was charged to the 25 mL three-necked round bottom flask, then 4 (770 mg, 2.48 mmol, 1 eq) and K₂CO₃ (1.03 g, 7.45 mmol, 3 eq) was added at 20° C. At 20° C., MeNH₂ (2 M, 6.21 mL, 3 eq) was added to the reaction mixture at 20° C. After the addition, the mixture was stirred at 20° C. for 12 hr. LCMS showed 4 was consumed completely and one main peak with desired m/z was detected. The mixture reaction was quenched by the addition of aq. FA (1 mol, 1 ml). The mixture was diluted with H₂O (40 mL), extracted by EA (30 mL*3). Then organic phase was washed by H₂O (20 mL*1) aq. brine (10 mL*1). The organic was dried over Na₂SO₄, filtered. The organic phase was concentrated under reduced pressure at 45° C. to give a residue. The residue was purified by column chromatography (SiO₂, Petroleum ether/Ethyl acetate=100/0 to 90/10). 5 (520 mg, 1.62 mmol, 65.21% yield) was obtained as a yellow solid. LCMS: RT=1.364 min, MS cal.: 320.02, 322.02, [M+H]⁺=320.09 1H NMR (400 MHz, DMSO-d6) δ=6.66 (d, J=1.6 Hz, 1H), 6.60 (d, J=1.6 Hz, 1H), 6.57-6.49 (m, 1H), 4.15 (s, 1H), 4.10 (s, 1H), 2.75 (d, J=4.8 Hz, 3H), 1.39 (s, 3H), 1.34 (s, 3H)

### General Procedure for Preparation of Intermediate 6:

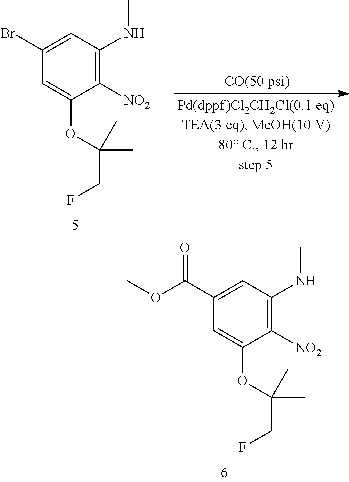 [SMILES: CNc1cc(Br)cc(OC(C)(C)CF)c1[N+](=O)[O-] ; CNc1cc(C(=O)OC)cc(OC(C)(C)CF)c1[N+](=O)[O-]]

MeOH (1 mL) and TEA (221.19 mg, 2.19 mmol, 304.26 μL, 3 eq) and Pd(dppf)Cl₂·CH₂Cl₂ (59.50 mg, 72.86 μmol, 0.1 eq) was charged to the 35 mL bottle, then 5 (234 mg, 728.65 μmol, 1 eq) was added at 20° C. After the addition, the mixture was degassed and purged with CO for 3 times. The mixture was stirred at 80° C. (50 psi) for 12 hr. LCMS showed 5 was consumed completely and one main peak with desired m/z was detected. TLC (Petroleum ether:Ethyl acetate=5:1) indicated 5 was consumed completely and one new spot formed. The reaction was clean according to TLC. After 12 hr, the reaction mixture was diluted with H₂O (20 mL), extracted by Ethyl acetate 20 mL (20 mL*2). Then organic phase was combined and washed by H₂O 10 mL, aq. brine 10 mL, dried with Na₂SO₄, filtered. The organic phase was concentrated under reduced pressure at 40° C. to give a residue. The residue was purified by column chromatography (SiO₂, Petroleum ether/Ethyl acetate=100/0 to 90/10). 6 (120 mg, 399.62 μmol, 54.84% yield) was obtained as a yellow solid.

¹H NMR (400 MHz, DMSO-d₆) δ=6.96 (d, J=1.6 Hz, 1H), 6.82-6.79 (m, 1H), 4.98-4.93 (m, 1H), 4.86 (s, 2H), 3.99 (d, J=19.6 Hz, 2H), 3.77 (s, 3H), 2.76 (d, J=4.8 Hz, 3H), 1.50 (s, 2H), 1.49-1.48 (m, 1H), 1.45 (s, 3H)

### General Procedure for Preparation of Intermediate 7:

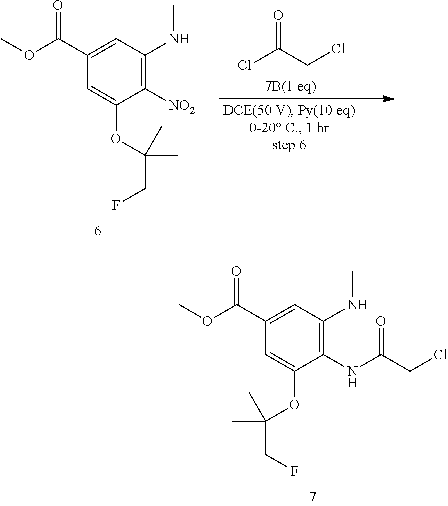 [SMILES: CNc1cc(C(=O)OC)cc(OC(C)(C)CF)c1[N+](=O)[O-] ; O=C(Cl)CCl ; CNc1cc(C(=O)OC)cc(OC(C)(C)CF)c1NC(=O)CCl]

DCE (1 mL) was charged to the 50 mL three-necked round bottom flask, then 6 (96 mg, 355.16 μmol, 1 eq) and Py (280.93 mg, 3.55 mmol, 286.67 μL, 10 eq) was added at 20° C. At 0° C., 6 (72.20 mg, 639.29 μmol, 50.92 μL, 1.8 eq) in DCE (1 mL) was added to the reaction mixture at 0° C. After the addition, the mixture was stirred at 20° C. for 1 hr. LCMS showed 6 was consumed completely and one main peak with desired m/z was detected and TLC (Petroleum ether:Ethyl acetate-0:1) indicated 6 was consumed completely and one new spot formed. The reaction was clean according to TLC. After 12 hr, the reaction mixture was diluted with H₂O (20 mL), extracted by Ethyl acetate 30 mL (10 mL*3). Then organic phase was combined and washed by H₂O 10 mL, aq. brine 10 mL, dried with Na₂SO₄, filtered. The organic phase was concentrated under reduced pressure at 40° C. to give a crude product. 7 (100 mg, 288.37 μmol, 81.19% yield) was obtained as a brown solid, the crude product was used to next step without further purification.

LCMS: RT=1.104 min, MS cal.: 346.11, 348.11, [M+H]⁺=346.9.

### General Procedure for Preparation of Intermediate 8:

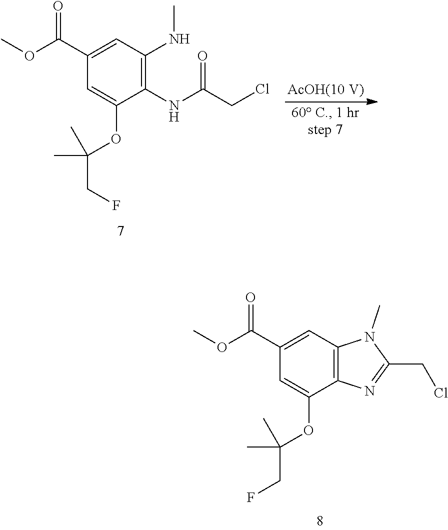 [SMILES: CNc1cc(C(=O)OC)cc(OC(C)(C)CF)c1NC(=O)CCl ; COC(=O)c1cc(OC(C)(C)CF)c2nc(CCl)n(C)c2c1]

To a solution of 7 (100 mg, 288.37 μmol, 1 eq) in AcOH (1 mL). The mixture was stirred at 60° C. for 1 hr. LCMS showed 7 was consumed completely and one main peak with desired m/z was detected. The reaction mixture was concentrated to dryness. 8 (94 mg, 285.92 μmol, 99.15% yield) was obtained as a brown solid, the crude product was used to next step without further purification.

LCMS: RT=1.127 min, MS cal.: 328.10, 330.10, [M+H]⁺=329.0

### General Procedure for Preparation of Intermediate 9:

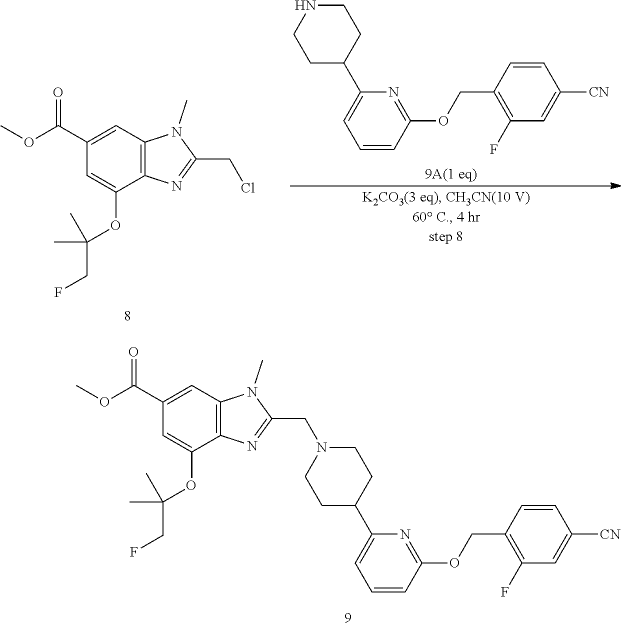 [SMILES: COC(=O)c1cc(OC(C)(C)CF)c2nc(CCl)n(C)c2c1 ; N#Cc1ccc(COc2cccc(C3CCNCC3)n2)c(F)c1 ; COC(=O)c1cc(OC(C)(C)CF)c2nc(CN3CCC(c4cccc(OCc5ccc(C#N)cc5F)n4)CC3)n(C)c2c1]

To a solution of 8 (94 mg, 285.92 μmol, 1 eq) and 9A (89.02 mg, 285.92 μmol, 1 eq) in CH₃CN (1 mL) was added K₂CO₃ (118.55 mg, 857.75 μmol, 3 eq). The mixture was stirred at 60° C. for 4 r. LCMS showed 8 was consumed completely and one main peak with desired m/z was detected. TLC (Petroleum ether:Ethyl acetate=0:1) indicated 8 was consumed completely and one new spot formed. The reaction was clean according to TLC. After 4 r, the reaction mixture was diluted with H₂O (20 mL), extracted by Ethyl acetate 40 mL (20 mL*2). Then organic phase was combined and washed by H₂O 10 mL, aq. brine 10 mL, dried with Na₂SO₄, filtered. The organic phase was concentrated under reduced pressure at 40° C. to give a residue. The residue was purified by column chromatography (SiO₂, Petroleum ether/Ethyl acetate=100/0 to 60/40). 9 (60 mg, 92.44 μmol, 32.33% yield, 93% purity) was obtained as a yellow solid.

LCMS: RT=1.565 min, MS cal.: 603.27, 604.27, [M+H]⁺=604.1

¹H NMR (400 MHz, DMSO-d₆) δ=7.80 (s, 1H), 7.68-7.60 (m, 1H), 7.57-7.51 (m, 1H), 7.45 (s, 2H), 7.37 (d, J=9.2 Hz, 1H), 6.81-6.74 (m, 1H), 6.69-6.62 (m, 1H), 5.50 (s, 2H), 4.32 (d, J=18.0 Hz, 2H), 4.13 (q, J=7.2 Hz, 2H), 4.03-3.98 (m, 2H), 3.97 (s, 3H), 3.07-2.87 (m, 2H), 2.69-2.54 (m, 1H), 2.32 (br s, 2H), 1.64 (br s, 4H), 1.61 (s, 6H), 1.48-1.47 (m, 1H), −0.82-−0.85 (m, 1H)

### General Procedure for Preparation of Compound 160 (10):

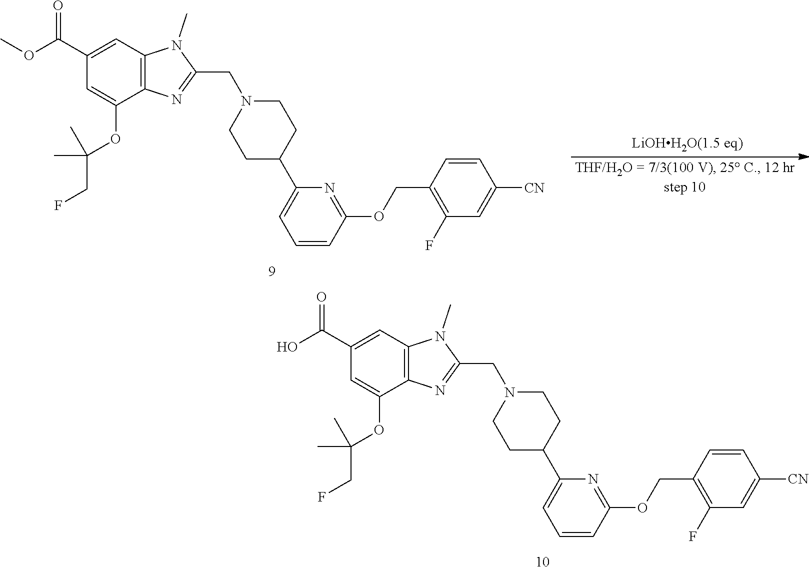 [SMILES: COC(=O)c1cc(OC(C)(C)CF)c2nc(CN3CCC(c4cccc(OCc5ccc(C#N)cc5F)n4)CC3)n(C)c2c1 ; Cn1c(CN2CCC(c3cccc(OCc4ccc(C#N)cc4F)n3)CC2)nc2c(OC(C)(C)CF)cc(C(=O)O)cc21]

To a solution of 9 (50 mg, 82.83 μmol, 1 eq) in THF (0.35 L) and H₂O (0.15 L) was added LiOH·H₂O (5.21 mg, 124.24 μmol, 1.5 eq). The mixture was stirred at 25° C. for 12 hr. The reaction was monitored by LCMS showed 9 was consumed completely and one main peak with desired m/z was detected. The residue was purified by prep-HPLC (column: Waters Xbridge BEH C18 100*30 mm*10 um; mobile phase: [H₂O (10 mM NH₄HCO₃)-ACN]; gradient: 20%-50% B over 8.0 min). 2-((4-(6-((4-Cyano-2-fluorobenzyl)oxy)pyridin-2-yl)piperidin-1-yl)methyl)-4-((1-fluoro-2-methylpropan-2-yl)oxy)-1-methyl-1H-benzo[d]imidazole-6-carboxylic acid (17.08 mg, 28.10 μmol, 33.92% yield, 97% purity) was obtained as a white solid.

LCMS: Rt=2.630 min, MS cal.: 589.25, 590.25, [M+H]⁺=590.3

HPLC: Rt=10.803 min, purity=97.00%

¹H NMR (400 MHz, MeOH-d4) δ=7.89 (d, J=10.0 Hz, 1H), 7.80 (s, 1H), 7.72-7.68 (m, 2H), 7.67-7.62 (m, 1H), 7.31 (s, 1H), 6.89 (d, J=7.2 Hz, 1H), 6.72 (d, J=8.0 Hz, 1H), 5.46 (s, 2H), 4.26 (s, 1H), 4.32 (s, 1H), 3.92 (s, 3H), 3.83 (s, 2H), 2.91 (br d, J=10.8 Hz, 2H), 2.62-2.55 (m, 1H), 2.20 (br t, J=10.8 Hz, 2H), 1.80-1.73 (m, 2H), 1.71-1.61 (m, 2H), 1.53-1.51 (m, 3H), 1.46 (s, 3H)

### Example A205—Preparation of Compound 136. 2-((4-(6-((4-Chloro-2-fluorobenzyl)oxy)pyridin-2-yl)piperidin-1-yl)methyl)-4-(difluoromethoxy)-1-methyl-1H-benzo[d]imidazole-6-carboxylic acid (Compound 136) (5)

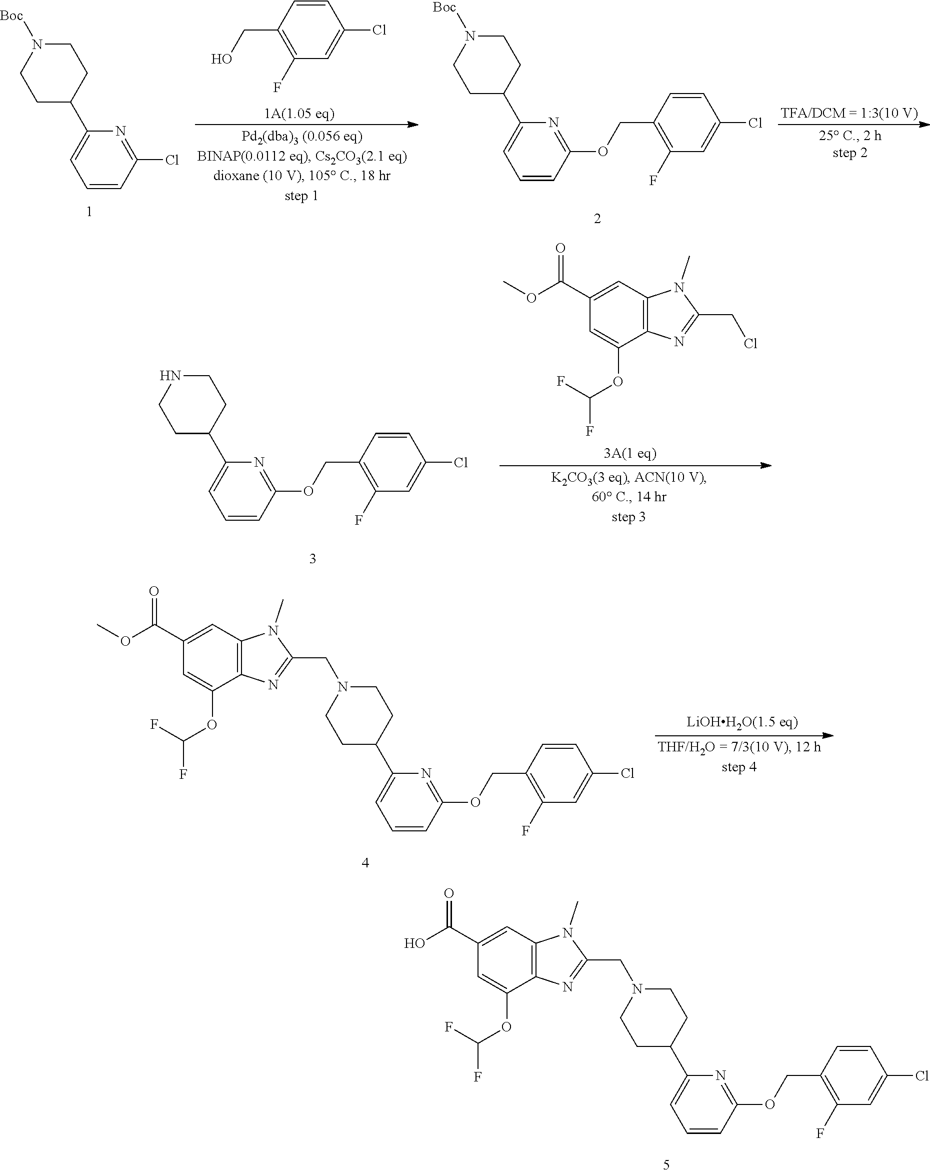

### General Procedure for Preparation of Intermediate 2:

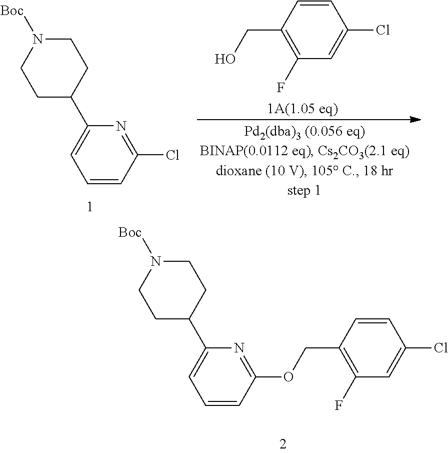 [SMILES: CC(C)(C)OC(=O)N1CCC(c2cccc(Cl)n2)CC1 ; OCc1ccc(Cl)cc1F ; CC(C)(C)OC(=O)N1CCC(c2cccc(OCc3ccc(Cl)cc3F)n2)CC1]

To a solution of 1 (5 g, 16.85 mmol, 1 eq) in dioxane (63 mL) was added 1A (2.84 g, 17.69 mmol, 1.05 eq), BINAP (1.17 g, 1.89 mmol, 0.112 eq), Cs₂CO₃ (11.53 g, 35.38 mmol, 2.1 eq) and Pd₂(dba)₃ (863.91 mg, 943.42 μmol, 0.056 eq). The mixture was stirred at 105° C. for 18 hr. LCMS showed 1 was consumed completely and one main peak with desired mass was detected. The reaction mixture was diluted with H₂O 250 mL and extracted with EtOAc 400 mL (100 mL*4). The combined organic layers were washed with brine 200 mL (100 mL*2), dried over Na₂SO₄, filtered and concentrated under reduced pressure to give a residue. The residue was purified by column chromatography (SiO₂, PE/EtOAc=1/0 to 94:/6). The residue was purified by prep-HPLC (neutral condition: column: Waters Xbridge BEH C18 250*50 mm*10 um; mobile phase: [H₂O (10 mM NH₄HCO₃)-ACN]; gradient: 55%-95% B over 10.0 min). 2 (6.5 g, 12.82 mmol, 76.08% yield) was obtained as a yellow solid.

LCMS: RT=1.744 min, MS cal.: 420.2/422.2, [M+H]⁺=421.0/423.0

¹HNMR (400 MHz, CHCl3-d) δ=7.52 (t, J=7.6 Hz, 1H), 7.47-7.42 (m, 2H), 7.16-7.09 (m, 2H), 6.73 (d, J=7.2 Hz, 1H), 6.62 (d, J=8.4 Hz, 1H), 5.41 (s, 2H), 4.29-4.16 (m, 2H), 2.89-2.68 (m, 3H), 1.87 (br d, J=12.8 Hz, 2H), 1.79-1.67 (m, 2H), 1.50 (s, 9H)

### General Procedure for Preparation of Intermediate 3:

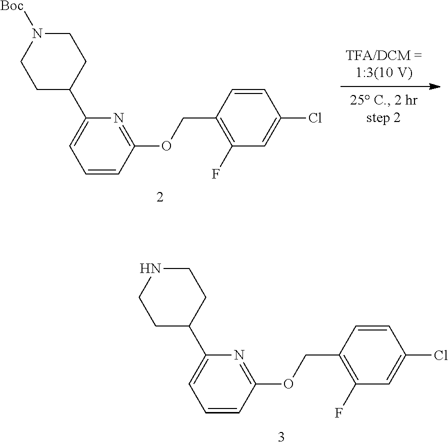 [SMILES: CC(C)(C)OC(=O)N1CCC(c2cccc(OCc3ccc(Cl)cc3F)n2)CC1 ; Fc1cc(Cl)ccc1COc1cccc(C2CCNCC2)n1]

A mixture of 2 (200 mg, 475.17 μmol, 1 eq), TFA (460.50 mg, 4.04 mmol, 300.00 μL, 8.50 eq) in DCM (1 mL) was degassed and purged with N₂ for 3 times, and then the mixture was stirred at 25° C. for 2 hr under N₂ atmosphere. LCMS showed 2 was consumed completely and one main peak with desired mass was detected. The mixture was filtered and concentrated in vacuum. 3 (150 mg, crude) was obtained as a brown oil.

LCMS: RT=0.443 min, MS cal.: 320.11/322.11, [M+H]⁺=311.0/313.0

### General Procedure for Preparation of Intermediate 4:

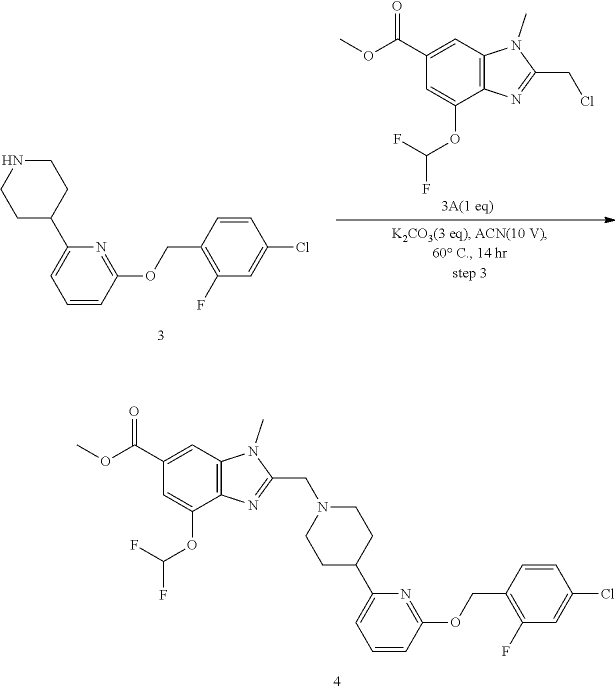 [SMILES: Fc1cc(Cl)ccc1COc1cccc(C2CCNCC2)n1 ; COC(=O)c1cc(OC(F)F)c2nc(CCl)n(C)c2c1 ; COC(=O)c1cc(OC(F)F)c2nc(CN3CCC(c4cccc(OCc5ccc(Cl)cc5F)n4)CC3)n(C)c2c1]

To a solution of 3 (130 mg, 405.25 μmol, 1 eq) in CH₃CN (1.3 mL) was added K₂CO₃ (168.02 mg, 1.22 mmol, 3 eq) and 3A (123.47 mg, 405.25 μmol, 1 eq). The mixture was stirred at 60° C. for 2 hr. LCMS showed 28% of 3 remained. Several new peaks were shown on LCMS and 45% of desired 4 as detected. The mixture was added K₂CO₃ (112.02 mg, 810.50 μmol, 2 eq). The mixture was stirred at 60° C. for 12 hr. LCMS 3 showed was consumed completely and 45% of desired 4 was detected. The reaction mixture was diluted with H₂O 5 mL and extracted with EtOAc 10 mL (5 mL*2). The combined organic layers were washed with brine 5 mL, dried over Na₂SO₄, filtered, and concentrated under reduced pressure to give a residue. The residue was purified by column chromatography (SiO₂, PE/EtOAc=1/0 to 1/1). 4 (140 mg, 216.30 μmol, 53.37% yield) was obtained as a white solid.

LCMS: RT=2.676 min, MS cal.: 588.2/590.2, [M+H]⁺=589.2/591.2

### General Procedure for Preparation of Compound 136 (5):

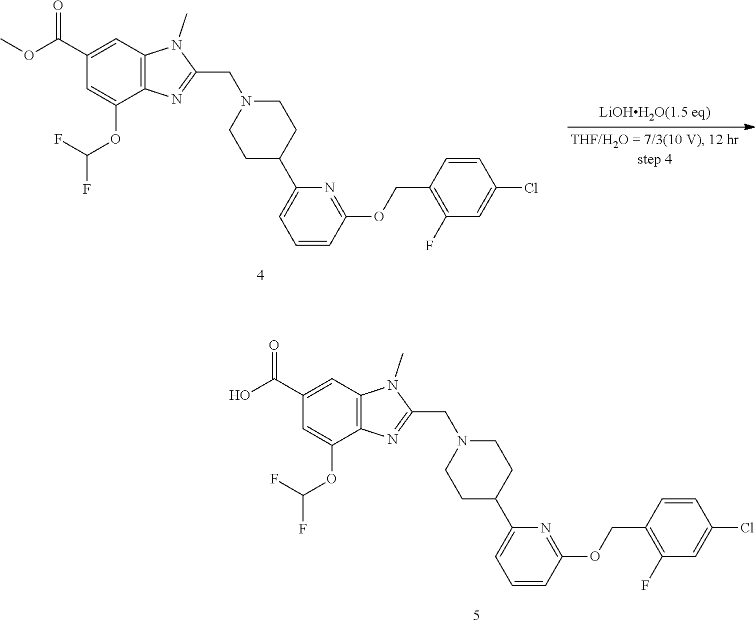 [SMILES: COC(=O)c1cc(OC(F)F)c2nc(CN3CCC(c4cccc(OCc5ccc(Cl)cc5F)n4)CC3)n(C)c2c1 ; Cn1c(CN2CCC(c3cccc(OCc4ccc(Cl)cc4F)n3)CC2)nc2c(OC(F)F)cc(C(=O)O)cc21]

To a solution of 4 (80 mg, 135.82 μmol, 1 eq) in THF (0.7 mL) and H₂O (0.3 mL) was added LiOH·H₂O (8.55 mg, 203.73 μmol, 1.5 eq). The mixture was stirred at 25° C. for 12 hr. LCMS showed 10% of 4 remained. Several new peaks were shown on LCMS and 83% of desired product was detected. The mixture was added CH₃CN 0.5 mL. The residue was purified by prep-HPLC (neutral condition: column: Waters Xbridge Prep OBD C18 150*40 mm*10 um; mobile phase: [H₂O (10 mM NH₄HCO₃)-ACN]; gradient: 15%-55% B over 8.0 min). 2-((4-(6-((4-Chloro-2-fluorobenzyl)oxy)pyridin-2-yl)piperidin-1-yl)methyl)-4-(difluoromethoxy)-1-methyl-1H-benzo[d]imidazole-6-carboxylic acid (28.40 mg, 48.41 μmol, 35.64% yield) was obtained as a white solid.

LCMS: RT=1.178 min, MS cal.: 574.2/576.2, [M+H]⁺=575.0/577.0

LCMS: RT=2.836 min, MS cal.: 574.2/576.2, [M+H]⁺=575.2/577.2

HPLC: RT=12.167 min

¹HNMR (400 MHz, MeOH-d4) δ=8.11 (s, 1H), 7.69 (s, 1H), 7.57 (t, J=8.0 Hz, 1H), 7.49 (t, J=8.0 Hz, 1H), 7.25 (t, J=74 Hz, 1H), 7.22-7.15 (m, 2H), 6.83 (d, J=7.2 Hz, 1H), 6.63 (d, J=8.4 Hz, 1H), 5.41 (s, 2H), 4.03 (s, 3H), 3.98 (s, 2H), 3.09 (br d, J=11.2 Hz, 2H), 2.71-2.62 (m, 1H), 2.45-2.36 (m, 2H), 1.93-1.85 (m, 4H)

### Example A206—Preparation of Compound 135. 4-(tert-Butoxy)-2-((4-(6-((4-cyano-2-fluorobenzyl)oxy)pyridin-2-yl)piperidin-1-yl)methyl)-1-methyl-1H-benzo[d]imidazole-6-carboxylic acid (Compound 135) (6)

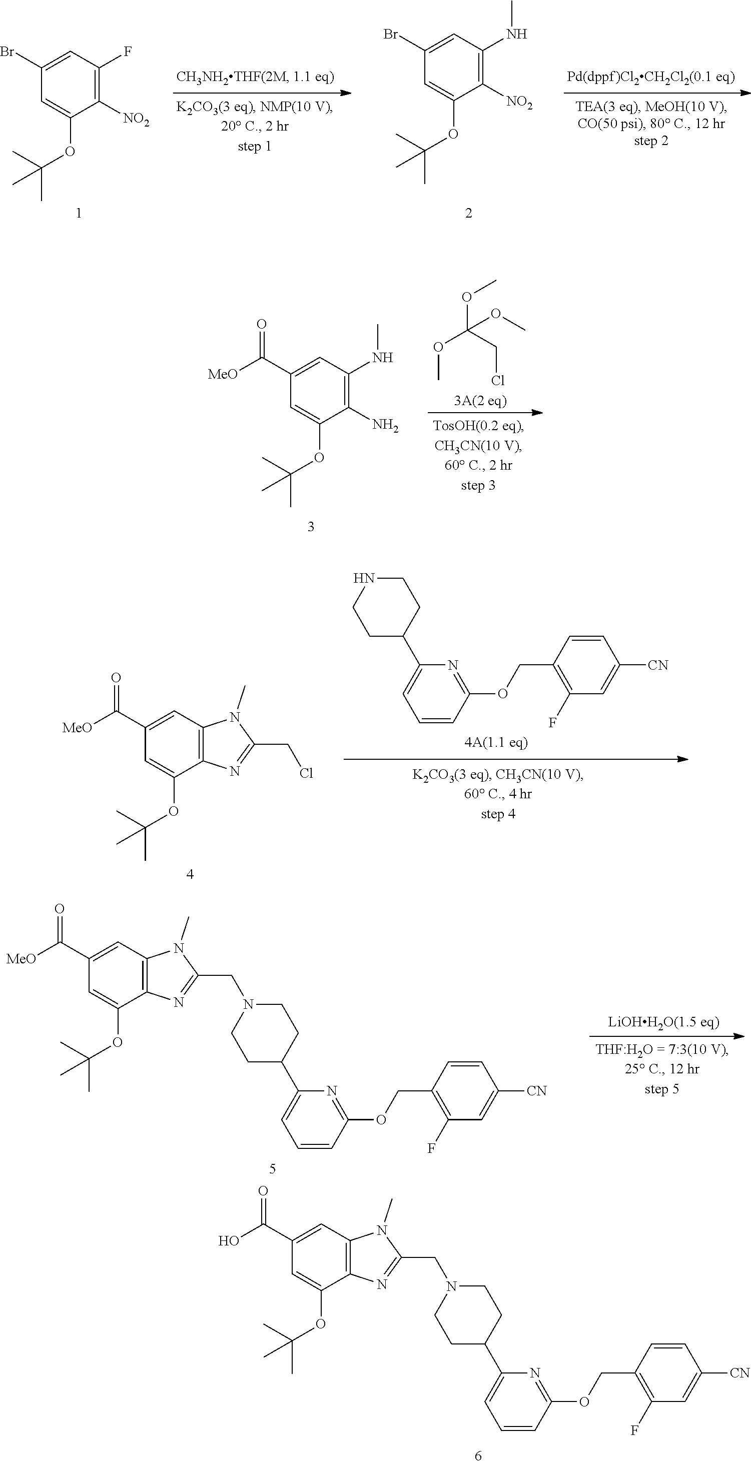

### General Procedure for Preparation of Intermediate 2:

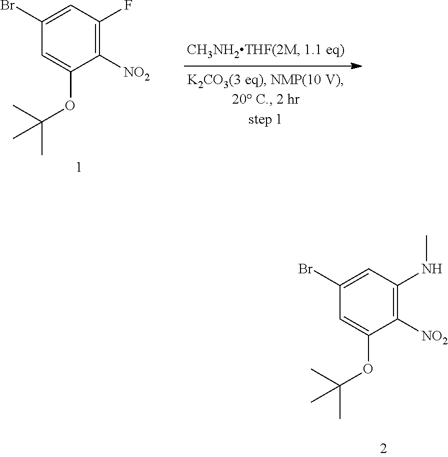 [SMILES: CC(C)(C)Oc1cc(Br)cc(F)c1[N+](=O)[O-] ; CNc1cc(Br)cc(OC(C)(C)C)c1[N+](=O)[O-]]

NMP (6 mL) was charged to the three-necked round bottom flask, then 1 (600 mg, 2.05 mmol, 1 eq), K₂CO₃ (851.68 mg, 6.16 mmol, 3 eq) was added to the mixture at 25° C. MeNH₂ (2 M, 1.13 mL, 1.1 eq) was added dropwise to the reaction mixture at 25° C. After the addition, the mixture was stirred at 25° C. for 2 hr. TLC indicated 1 was consumed completely and one new spot formed. The reaction mixture was adjusted to pH-5 with FA (1 M) at 20° C. and diluted with H₂O (20 mL), extracted with EtOAc (30 mL). The combined organic layers were washed with brine (20 mL), dried over Na₂SO₄, filtered. The organic phase was concentrated under reduced pressure at 40° C. to give a residue. The residue was purified by column chromatography (SiO₂, Petroleum ether/Ethyl acetate=100/1 to 10/1). 2 (523 mg, 1.73 mmol, 83.99% yield) was obtained as a yellow solid.

¹H NMR (400 MHz, DMSO-d₆) δ=6.62 (dd, J=1.6, 7.2 Hz, 2H), 6.28 (br d, J=4.4 Hz, 1H), 2.71 (d, J=4.8 Hz, 3H), 1.35-1.29 (m, 9H)

### General Procedure for Preparation of Intermediate 3:

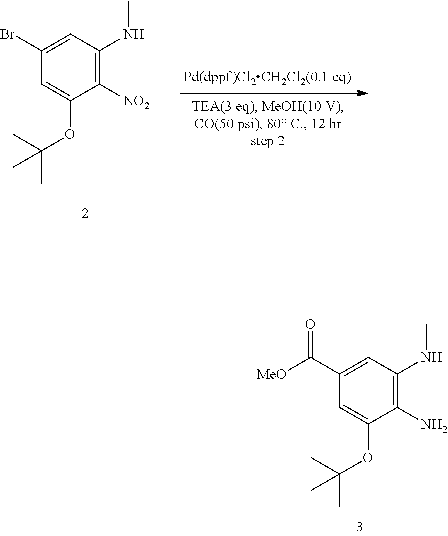 [SMILES: CNc1cc(Br)cc(OC(C)(C)C)c1[N+](=O)[O-] ; CNc1cc(C(=O)OC)cc(OC(C)(C)C)c1N]

MeOH (5 mL) was charged to the hydrogenating flask, then 2 (500 mg, 1.65 mmol, 1 eq) and TEA (500.68 mg, 4.95 mmol, 688.70 μL, 3 eq) was added to the mixture at 25° C. Pd(dppf)Cl₂·CH₂Cl₂ (134.69 mg, 164.93 μmol, 0.1 eq) was added to the reaction mixture at 25° C. After the addition, the mixture was stirred at 80° C., CO (50 psi) for 12 hr. TLC indicated 2 was consumed completely and one new spot formed. The reaction mixture was added to H₂O (10 mL) at 25° C. The mixture was extracted by EtOAc (60 mL). Then organic phase was combined and washed by brine (20 mL), dried with Na₂SO₄, filtered. The organic phase was concentrated under reduced pressure at 40° C. to give a residue. The residue was purified by column chromatography (SiO₂, Petroleum ether/Ethyl acetate=10/1 to 2/1). 3 (200 mg, 792.68 μmol, 48.06% yield) was obtained as a black oil.

¹H NMR (400 MHz, DMSO-d₆) δ=7.00 (d, J=1.6 Hz, 1H), 6.78 (d, J=1.6 Hz, 1H), 4.90 (q, J=4.8 Hz, 1H), 4.86 (s, 2H), 3.74 (s, 3H), 2.74 (d, J=4.8 Hz, 3H), 1.32 (s, 9H)

### General Procedure for Preparation of Intermediate 4:

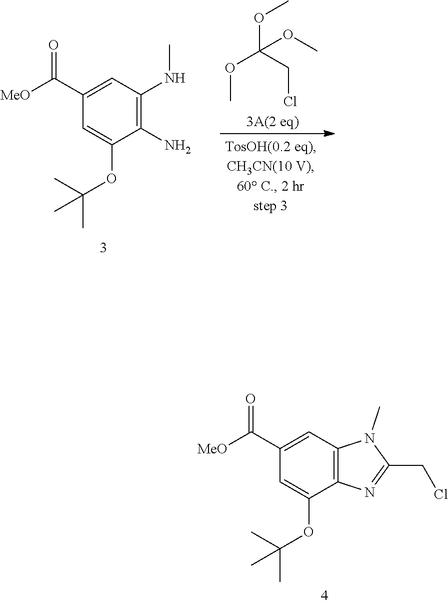

A mixture of 3 (130 mg, 515.24 μmol, 1 eq), 3A (159.30 mg, 1.03 mmol, 138.89 μL, 2 eq), TosOH (8.87 mg, 51.52 μmol, 0.1 eq) in CH₃CN (1 mL) was degassed and purged with N₂ for 3 times, and then the mixture was stirred at 60° C. for 2 hr under N₂ atmosphere. LC-MS showed 3 was consumed completely and desired mass was detected. The reaction mixture was concentrated under reduced pressure to remove solvent. 4 (160 mg, 514.84 μmol, 99.92% yield) was obtained as a yellow oil.

LCMS: RT=1.456 min, MS cal.: 310.1, 312.1, [M+H]⁺=311.1

¹H NMR (400 MHz, DMSO-d₆) δ=7.97 (d, J=1.2 Hz, 1H), 7.43 (d, J=1.2 Hz, 1H), 5.12 (s, 2H), 3.91 (s, 3H), 3.88 (s, 3H), 1.40 (s, 9H)

### General Procedure for Preparation of Intermediate 5:

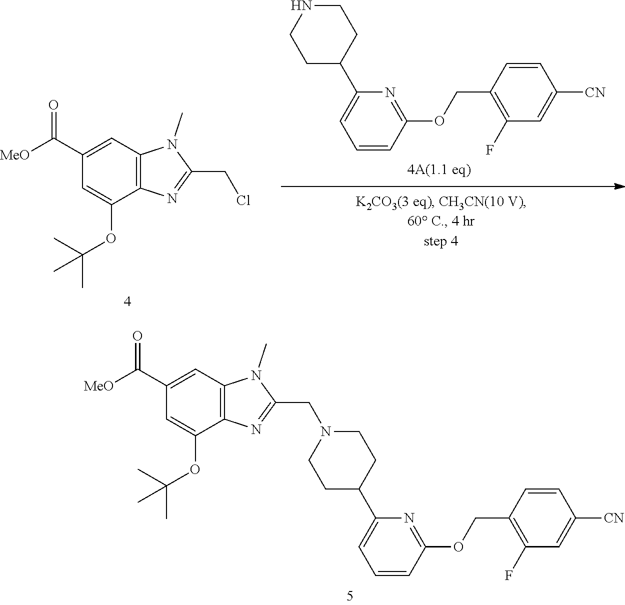 [SMILES: COC(=O)c1cc(OC(C)(C)C)c2nc(CCl)n(C)c2c1 ; N#Cc1ccc(COc2cccc(C3CCNCC3)n2)c(F)c1 ; COC(=O)c1cc(OC(C)(C)C)c2nc(CN3CCC(c4cccc(OCc5ccc(C#N)cc5F)n4)CC3)n(C)c2c1]

To a solution of 4 (180 mg, 579.20 μmol, 1 eq), 4A (198.37 mg, 637.12 μmol, 1.1 eq) in CH₃CN (2 mL) was added K₂CO₃ (240.14 mg, 1.74 mmol, 3 eq). The mixture was stirred at 60° C. for 4 hr. LC-MS showed 4 was consumed completely and desired mass was detected. The reaction mixture was diluted with H₂O (10 mL) and extracted with EtOAc 15 mL (5 mL*3). The combined organic layers were washed with brine (20 mL), dried over Na₂SO₄, filtered, and concentrated under reduced pressure to give a residue. The residue was purified by column chromatography (SiO₂, Petroleum ether/Ethyl acetate=2/1 to 0/1). 5 (180 mg, 307.34 μmol, 53.06% yield) was obtained as a yellow oil.

LCMS: RT=0.476 min, MS cal.: 585.3, [M+H]⁺=586.4

¹H NMR (400 MHz, DMSO-d₆) δ=7.91 (d, J=1.2 Hz, 1H), 7.87 (d, J=10.0 Hz, 1H), 7.68 (d, J=3.6 Hz, 2H), 7.63 (t, J=7.6 Hz, 1H), 7.38 (d, J=1.3 Hz, 1H), 6.86 (d, J=7.2 Hz, 1H), 6.71 (d, J=8.0 Hz, 1H), 5.44 (s, 2H), 3.91 (s, 3H), 3.87 (s, 3H), 3.81 (s, 2H), 2.90 (br d, J=11.2 Hz, 2H), 2.61-2.54 (m, 1H), 2.19 (br t, J=10.4 Hz, 2H), 1.79-1.58 (m, 4H), 1.38 (s, 10H)

### General Procedure for Preparation of Compound 135 (6):

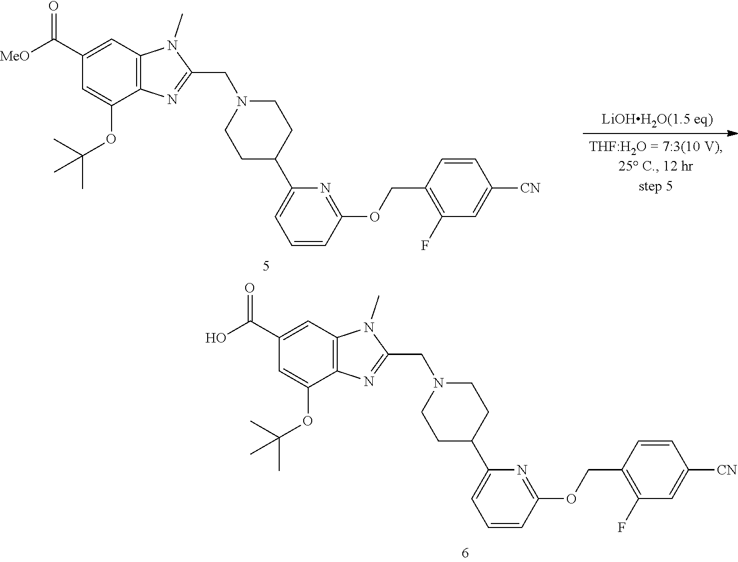 [SMILES: COC(=O)c1cc(OC(C)(C)C)c2nc(CN3CCC(c4cccc(OCc5ccc(C#N)cc5F)n4)CC3)n(C)c2c1 ; Cn1c(CN2CCC(c3cccc(OCc4ccc(C#N)cc4F)n3)CC2)nc2c(OC(C)(C)C)cc(C(=O)O)cc21]

To a solution of 5 (180 mg, 307.34 μmol, 1 eq) in THF (0.7 mL) was added LiOH H₂O (19.35 mg, 461.01 μmol, 1.5 eq) in H₂O (0.3 mL). The mixture was stirred at 25° C. for 12 hr. LC-MS showed ˜ 50% of 5 remained. One new peak was shown on LC-MS and ˜43% of desired

Compound was detected. The reaction mixture was purified directly. The residue was purified by prep-HPLC (neutral condition; column: Waters Xbridge Prep OBD C18 150*40 mm*10 um; mobile phase: [H₂O (10 mM NH₄HCO₃)-ACN]; gradient: 25%-75% B over 8.0 min). 4-(tert-Butoxy)-2-((4-(6-((4-cyano-2-fluorobenzyl)oxy)pyridin-2-yl)piperidin-1-yl)methyl)-1-methyl-1H-benzo[d]imidazole-6-carboxylic acid (29.61 mg, 50.76 μmol, 16.52% yield, 98% purity) was obtained as a white solid.

LCMS: RT=2.640 min, MS cal.: 571.3, [M+H]⁺=572.3

HPLC: RT=10.911 min, purity: 98.7%

¹H NMR (400 MHz, MeOH-d4) δ=7.96 (d, J=1.2 Hz, 1H), 7.69-7.64 (m, 1H), 7.63-7.50 (m, 4H), 6.83 (d, J=7.2 Hz, 1H), 6.68 (d, J=8.0 Hz, 1H), 5.50 (s, 2H), 3.98 (s, 3H), 3.95 (s, 2H), 3.06 (br d, J=11.2 Hz, 2H), 2.71-2.57 (m, 1H), 2.43-2.30 (m, 2H), 1.91-1.79 (m, 4H), 1.47 (s, 9H)

### Example A207—Preparation of Compound 128. 4-Acetoxy-2-((4-(6-((4-cyano-2-fluorobenzyl)oxy)pyridin-2-yl)piperidin-1-yl)methyl)-1-methyl-1H-benzo[d]imidazole-6-carboxylic acid (Compound 128) (2)

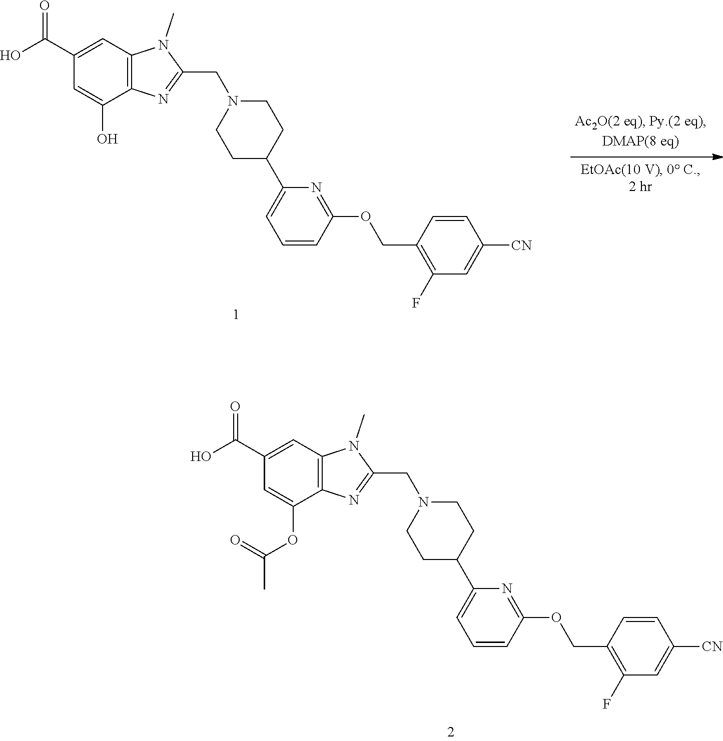 [SMILES: Cn1c(CN2CCC(c3cccc(OCc4ccc(C#N)cc4F)n3)CC2)nc2c(O)cc(C(=O)O)cc21 ; CC(=O)Oc1cc(C(=O)O)cc2c1nc(CN1CCC(c3cccc(OCc4ccc(C#N)cc4F)n3)CC1)n2C]

### General Procedure for Preparation of Compound 128 (2):

To a solution of 1 (70 mg, 135.78 μmol, 1 eq) in EtOAc (0.5 mL) was added Ac₂O (34.65 mg, 339.45 μmol, 31.88 μL, 2.5 eq), DMAP (132.70 mg, 1.09 mmol, 8 eq) and Py. (21.48 mg, 271.56 μmol, 21.92 μL, 2 eq) at 0° C. for 1 hr. The mixture was stirred at 25° C. for 12 hr. LCMS showed 1 was consumed completely. The reaction mixture was added with 1M FA to adjust pH=5 and extracted with EtOAc (5 mL*3). The combined organic layers were washed with aq. NaHCO₃ (3 mL*3), dried over Na₂SO₄, filtered, and concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 15%-65% B over 8.0 min). 4-Acetoxy-2-((4-(6-((4-cyano-2-fluorobenzyl)oxy)pyridin-2-yl)piperidin-1-yl)methyl)-1-methyl-1H-benzo[d]imidazole-6-carboxylic acid (25.1 mg, 40.47 μmol, 29.81% yield, 89.9% purity) was obtained as a white solid.

LCMS: RT=2.558 min, MS cal.: 557.5, [M+H]⁺=558.2

HPLC: RT=10.226 min, purity: 89.90%

¹H NMR (400 MHz, DMSO-d6) δ=8.08 (d, J=1.2 Hz, 1H), 7.88 (d, J=10.4 Hz, 1H), 7.72-7.68 (m, 2H), 7.64 (t, J=8.0 Hz, 1H), 7.51 (d, J=1.2 Hz, 1H), 6.87 (d, J=7.2 Hz, 1H), 6.71 (d, J=8.4 Hz, 1H), 5.45 (s, 2H), 3.96 (s, 3H), 3.82 (s, 2H), 2.89 (br d, J=11.2 Hz, 2H), 2.58-2.54 (m, 1H), 2.37 (s, 3H), 2.22-2.15 (m, 2H), 1.78-1.62 (m, 4H)

### Example A208—Preparation of Compound 127. 2-((4-(6-((4-Cyano-2-fluorobenzyl)oxy)pyridin-2-yl)piperidin-1-yl)methyl)-4-(difluoromethoxy)-1-methyl-1H-benzo[d]imidazole-6-carboxylic acid (Compound 127) (3)

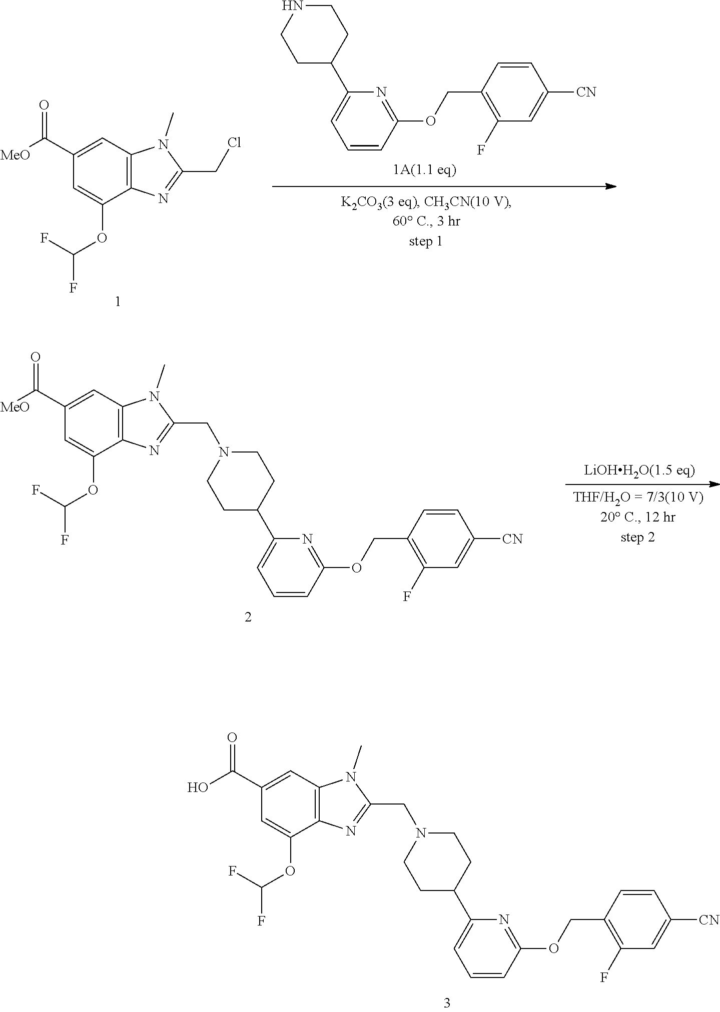 [SMILES: COC(=O)c1cc(OC(F)F)c2nc(CCl)n(C)c2c1 ; N#Cc1ccc(COc2cccc(C3CCNCC3)n2)c(F)c1 ; COC(=O)c1cc(OC(F)F)c2nc(CN3CCC(c4cccc(OCc5ccc(C#N)cc5F)n4)CC3)n(C)c2c1 ; Cn1c(CN2CCC(c3cccc(OCc4ccc(C#N)cc4F)n3)CC2)nc2c(OC(F)F)cc(C(=O)O)cc21]

### General Procedure for Preparation of Intermediate 2:

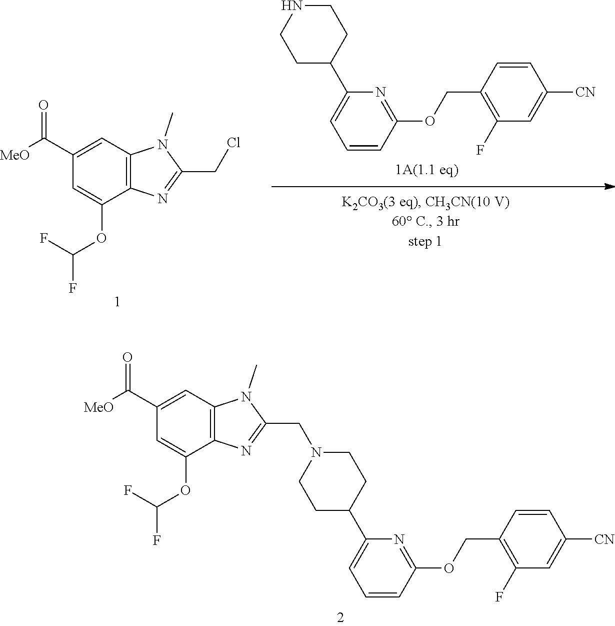 [SMILES: COC(=O)c1cc(OC(F)F)c2nc(CCl)n(C)c2c1 ; N#Cc1ccc(COc2cccc(C3CCNCC3)n2)c(F)c1 ; COC(=O)c1cc(OC(F)F)c2nc(CN3CCC(c4cccc(OCc5ccc(C#N)cc5F)n4)CC3)n(C)c2c1]

A mixture of 1 (100 mg, 328.22 μmol, 1 eq), 1A (112.41 mg, 361.04 μmol, 1.1 eq), K₂CO₃ (136.08 mg, 984.65 μmol, 3 eq) in CH₃CN (1 mL) was degassed and purged with N₂ for 3 times, and then the mixture was stirred at 60° C. for 3 hr under N₂ atmosphere. LCMS showed 1 was consumed completely and one main peak with desired mass was detected. The reaction mixture was diluted with H₂O (10 mL) and extracted with EtOAc (10 mL*3). The combined organic layers were washed with brine (5 mL*3), dried over Na₂SO₄, filtered, and concentrated under reduced pressure to give a residue. The residue was purified by column chromatography (SiO₂, Petroleum ether/Ethyl acetate=10/1 to 0/1). 2 (170 mg, 293.32 μmol, 89.37% yield) was obtained as a white solid.

LCMS: RT=0.741 min, MS cal.: 579.2, [M+H]⁺=580.2

¹H NMR (400 MHz, MeOH-d4) δ=8.13 (d, J=1.2 Hz, 1H), 7.69-7.63 (m, 2H), 7.62-7.50 (m, 3H), 7.25 (t, J=74.4 Hz, 1H), 6.82 (d, J=7.2 Hz, 1H), 6.66 (d, J=8.4 Hz, 1H), 5.52-5.48 (m, 2H), 4.03 (s, 3H), 3.95 (s, 3H), 3.89 (s, 2H), 2.98 (br d, J=11.6 Hz, 2H), 2.66-2.56 (m, 1H), 2.29 (br d, J=3.6 Hz, 2H), 1.81 (br dd, J=2.8, 7.6 Hz, 4H)

### General Procedure for Preparation of Compound 127 (3):

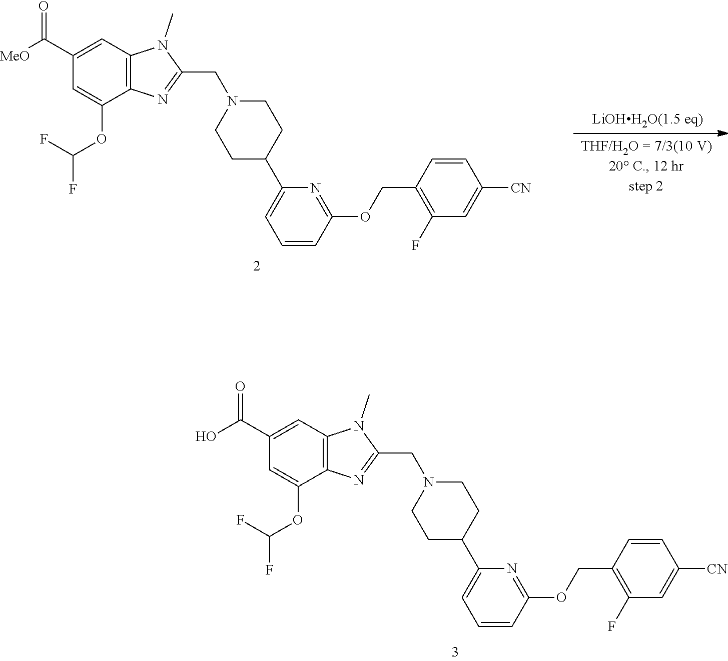 [SMILES: COC(=O)c1cc(OC(F)F)c2nc(CN3CCC(c4cccc(OCc5ccc(C#N)cc5F)n4)CC3)n(C)c2c1 ; Cn1c(CN2CCC(c3cccc(OCc4ccc(C#N)cc4F)n3)CC2)nc2c(OC(F)F)cc(C(=O)O)cc21]

A mixture of 2 (160 mg, 276.07 μmol, 1 eq), LiOH·H₂O (17.38 mg, 414.10 μmol, 1.5 eq) in THF (0.7 mL) and H₂O (0.3 mL) was degassed and purged with N₂ for 3 times, and then the mixture was stirred at 25° C. for 12 hr under N₂ atmosphere. LCMS showed 2 was consumed completely and one main peak with desired mass was detected. The reaction was added with 1M citric acid to adjust pH=8. The residue was purified by prep-HPLC (column: Waters Xbridge Prep OBD C18 150*40 mm*10 um; mobile phase: [H₂O (10 mM NH₄HCO₃)-ACN]; gradient: 20%-70% B over 8.0 min). 2-((4-(6-((4-Cyano-2-fluorobenzyl)oxy)pyridin-2-yl)piperidin-1-yl)methyl)-4-(difluoromethoxy)-1-methyl-1H-benzo[d]imidazole-6-carboxylic acid (27.56 mg, 45.42 μmol, 16.45% yield, 93.21% purity) was obtained as a white solid.

LCMS: RT=2.669 min, MS cal.: 565.5, [M+H]⁺=566.2

HPLC: RT=10.637 min, purity: 93.21%

¹H NMR (400 MHz, DMSO-d6) δ=8.06 (d, J=1.2 Hz, 1H), 7.88 (d, J=10.0 Hz, 1H), 7.72-7.67 (m, 2H), 7.69 (t, J=70.0 Hz, 1H), 7.65 (d, J=8.0 Hz, 1H), 7.52 (s, 1H), 6.88 (d, J=7.2 Hz, 1H), 6.71 (d, J=8.4 Hz, 1H), 5.45 (s, 2H), 3.96 (s, 3H), 3.84 (s, 2H), 2.92 (br d, J=11.2 Hz, 2H), 2.62-2.56 (m, 1H), 2.20 (br t, J=10.4 Hz, 2H), 1.80-1.63 (m, 4H)

### Example A209—Preparation of Compound 125. 2-((4-(6-((4-Cyano-2-fluorobenzyl)oxy)pyridin-2-yl)piperidin-1-yl)methyl)-1-methyl-4-(trifluoromethoxy)-1H-benzo[d]imidazole-6-carboxylic acid (Compound 125) (11)

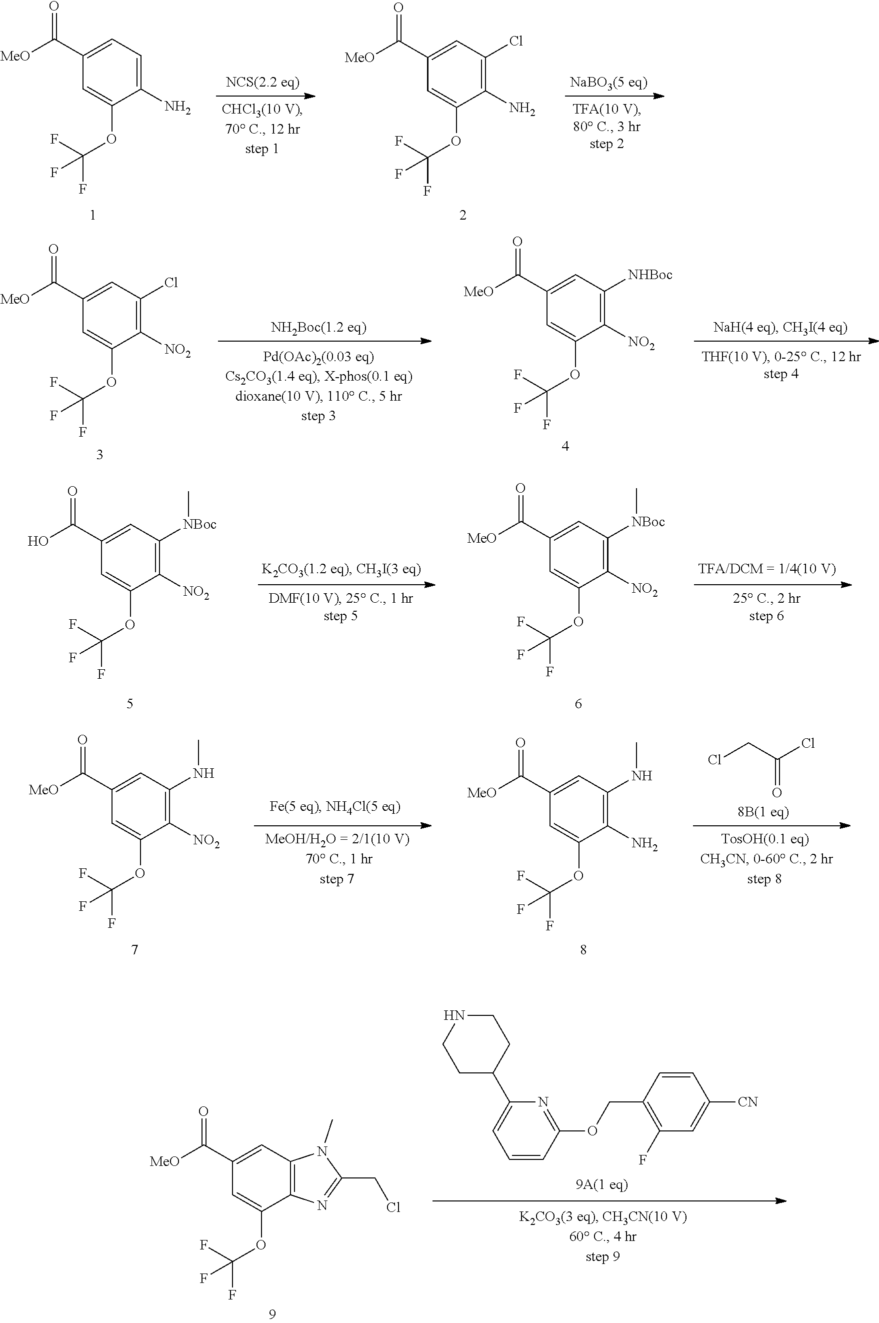

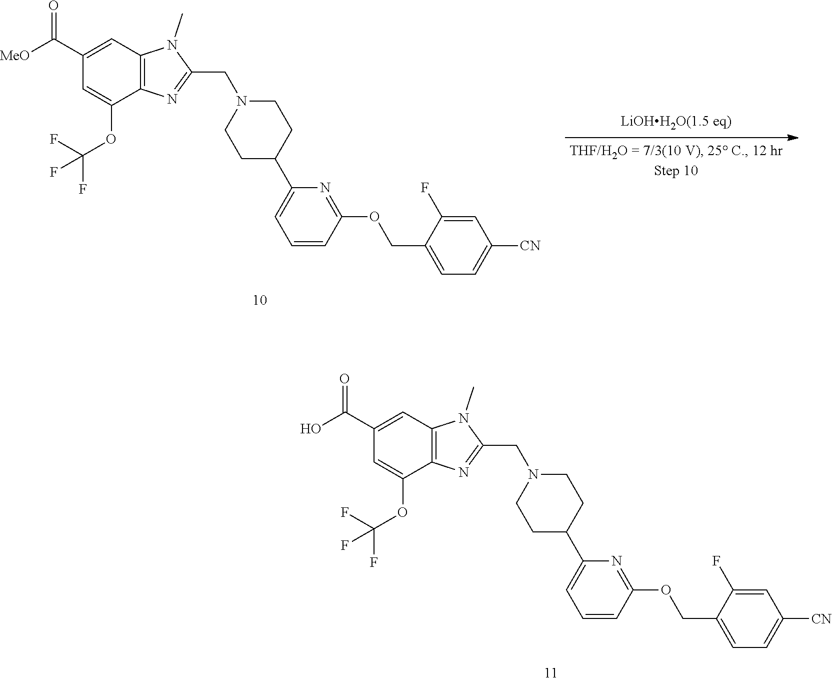 [SMILES: COC(=O)c1cc(OC(F)(F)F)c2nc(CN3CCC(c4cccc(OCc5ccc(C#N)cc5F)n4)CC3)n(C)c2c1 ; Cn1c(CN2CCC(c3cccc(OCc4ccc(C#N)cc4F)n3)CC2)nc2c(OC(F)(F)F)cc(C(=O)O)cc21]

### General Procedure for Preparation of Intermediate 2:

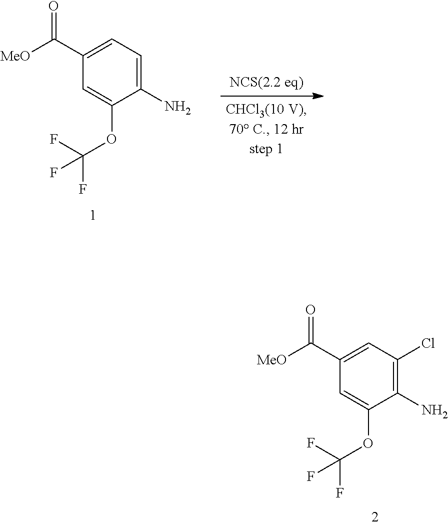 [SMILES: COC(=O)c1ccc(N)c(OC(F)(F)F)c1 ; COC(=O)c1cc(Cl)c(N)c(OC(F)(F)F)c1]

A mixture of 1 (2 g, 8.50 mmol, 1 eq), NCS (1.25 g, 9.36 mmol, 1.1 eq), K₂CO₃ (397.44 mg, 2.88 mmol, 3 eq) in CHCl₃ (20 mL) was degassed and purged with N₂ for 3 times, and then the mixture was stirred at 70° C. for 6 hrs under N₂ atmosphere. After 6 hrs, NCS (1.25 g, 9.36 mmol, 1.1 eq) was added to the reaction mixture at 70° C. and then the mixture was stirred at 70° C. for 6 hrs under N₂ atmosphere. TLC indicated 1 was consumed completely and one new spot formed (TLC-Ethyl acetate/Methanol=10/1, Product R_{f}=0.41). The reaction was clean according to TLC. The residue mixture was diluted with H₂O (100 mL) and extracted with DCM (100 mL*3). The combined organic layers were washed with brine (100 mL), dried over Na₂SO₄, filtered, and concentrated under reduced pressure to give a residue. The residue was purified by column chromatography (SiO₂, Petroleum ether/Ethyl acetate=100/1 to 9/1, TLC-Petroleum ether/Ethyl acetate=10/1, Product Rr-0.41). 2 (1.75 g, 6.43 mmol, 75.56% yield, 99% purity) was obtained as a white solid.

LCMS: RT=1.243 min, MS cal.: 269.01, [M−H]⁻=267.9

¹H NMR (400 MHz, CHCl3-d) δ=7.93 (d, J=1.6 Hz, 1H), 7.79-7.77 (m, 1H), 4.71 (br s, 2H), 3.89 (s, 3H)

### General Procedure for Preparation of Intermediate 3:

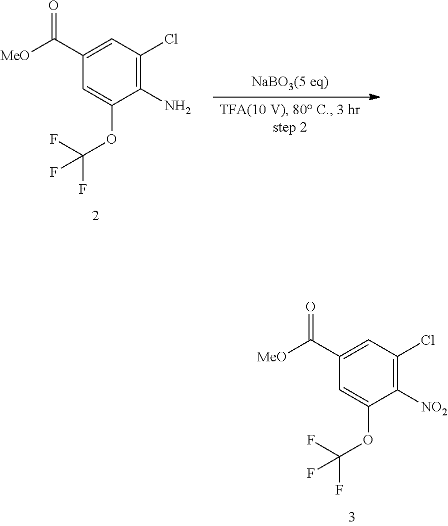 [SMILES: COC(=O)c1cc(Cl)c(N)c(OC(F)(F)F)c1 ; "NaBO3(5 eq)" ; "TFA(10 V), 80° C., 3 hrstep 2" ; COC(=O)c1cc(Cl)c([N+](=O)[O-])c(OC(F)(F)F)c1]

A mixture 2 (1.75 g, 6.49 mmol, 1 eq), NaBO₃ (4.99 g, 32.45 mmol, 6.24 mL, 5 eq) in TFA (18 mL) was degassed and purged with N₂ for 3 times, and then the mixture was stirred at 80° C. for 3 hrs under N₂ atmosphere. LC-MS showed 2 was consumed completely and one main peak with desired mass was detected. The residue mixture was diluted with H₂O (100 mL) and extracted with EtOAc (100 mL*3). The combined organic layers were washed with brine (100 mL), dried over Na₂SO₄, filtered and concentrated under reduced pressure to give a residue. The residue was purified by column chromatography (SiO₂, Petroleum ether/Ethyl acetate=100/1 to 9/1, TLC-Petroleum ether/Ethyl acetate=10/1, Product R_{f}=0.60). 3 (1.56 g, 5.12 mmol, 78.84% yield, 98.28% purity) was obtained as a white solid.

LCMS: RT=1.372 min, MS cal.: 298.98

¹H NMR (400 MHz, CHLOROFORM-d) δ=6.67 (d, J=1.6 Hz, 1H), 6.63 (d, J=1.6 Hz, 1H), 5.11-5.02 (m, 1H), 4.76-4.68 (m, 1H), 4.58 (td, J=6.0, 9.2 Hz, 1H), 3.39 (d, J=4.4 Hz, 2H), 2.79-2.67 (m, 1H), 2.55 (tdd, J=7.2, 9.1, 11.2 Hz, 1H), 1.42 (s, 9H)

### General Procedure for Preparation of Intermediate 4:

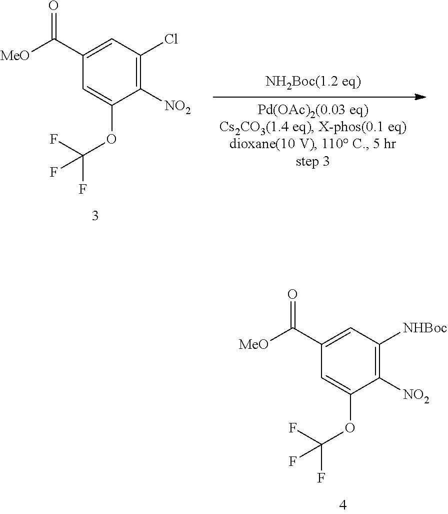

A mixture of 3 (1 g, 3.34 mmol, 1 eq), NH₂Boc (469.23 mg, 4.01 mmol, 1.2 eq), Cs₂CO₃ (1.52 g, 4.67 mmol, 1.4 eq), Pd(OAc)₂ (22.48 mg, 100.14 μmol, 0.03 eq) and X-phos (159.12 mg, 333.79 μmol, 0.1 eq) in dioxane (10 mL) was degassed and purged with N₂ for 3 times, and then the mixture was stirred at 110° C. for 5 hrs under N₂ atmosphere. LC-MS showed 3 was consumed completely and one main peak with desired mass was detected. The residue mixture was diluted with H₂O (50 mL) and extracted with EtOAc (50 mL*3). The combined organic layers were washed with brine (50 mL), dried over Na₂SO₄, filtered and concentrated under reduced pressure to give a residue. The residue was purified by column chromatography (SiO₂, Petroleum ether/Ethyl acetate=100/1 to 9/1, TLC-Petroleum ether/Ethyl acetate=10/1, Product R_{f}=0.37). 4 (500 mg, 1.30 mmol, 38.89% yield, 98.72% purity) was obtained as a white solid.

LCMS: RT=1.444 min, MS cal.: 380.08, [M−H]⁻=378.9

¹H NMR (400 MHz, CHLOROFORM-d) δ=8.88-8.84 (m, 1H), 7.73 (s, 1H), 7.58 (s, 1H), 3.99 (s, 3H), 1.54 (s, 9H)

### General Procedure for Preparation of Intermediate 5:

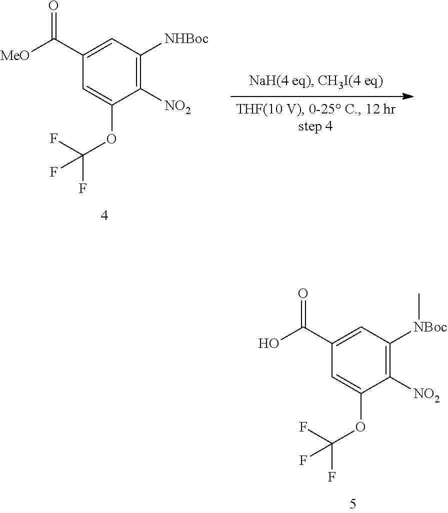 [SMILES: COC(=O)c1cc(NC(=O)OC(C)(C)C)c([N+](=O)[O-])c(OC(F)(F)F)c1 ; "NaH(4 eq), CH3I(4 eq)" ; "THF(10 V), 0-25° C., 12 hrstep 4" ; CN(C(=O)OC(C)(C)C)c1cc(C(=O)O)cc(OC(F)(F)F)c1[N+](=O)[O-]]

THF (5 mL) as charged to the three-necked round bottom flask, then 4 (500 mg, 1.31 mmol, 1 eq) was added to the mixture at 20° C. The mixture was degassed and purged with N₂ for 3 times, and then Cooled to 0° C. NaH (210.38 mg, 5.26 mmol, 60% purity, 4 eq) was added to the reaction mixture at 0° C. and stirred at 25° C. for 0.5 hr. Cooled to 0° C. CH₃I (746.52 mg, 5.26 mmol, 327.42 μL, 4 eq) was added dropwise to the reaction mixture at 0° C. and stirred at 25° C. for 11.5 hr. LC-MS showed 4 was consumed completely and one main peak with desired mass was detected. The reaction mixture was filtered and concentrated under reduced pressure to give a residue. The residue was diluted with H₂O (10 mL) and adjusted to pH=3-4 with FA (IM). The mixture was extracted with EtOAc (20 mL*3). The reaction mixture was poured into separatory funnel and separated. The combined organic layers were washed with brine (20 mL), dried over Na₂SO₄, filtered and concentrated under reduced pressure to give a residue. 5 (490 mg, crude) was obtained as a white solid.

LCMS: RT=0.989 min, MS cal.: 380.08, [M−H]⁻=378.9

### General Procedure for Preparation of Intermediate 6:

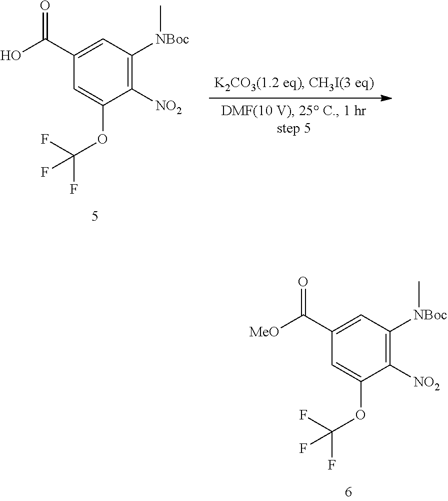 [SMILES: CN(C(=O)OC(C)(C)C)c1cc(C(=O)O)cc(OC(F)(F)F)c1[N+](=O)[O-] ; "DMF(10 V), 25° C., 1 hrstep 5" ; "K2CO3(1.2 eq), CH3I(3 eq)" ; COC(=O)c1cc(OC(F)(F)F)c([N+](=O)[O-])c(N(C)C(=O)OC(C)(C)C)c1]

A mixture of 5 (490 mg, 1.29 mmol, 1 eq), K₂CO₃ (213.70 mg, 1.55 mmol, 1.2 eq), CH₃I (548.68 mg, 3.87 mmol, 240.65 μL, 3 eq) in DMF (5 mL) was degassed and purged with N₂ for 3 times, and then the mixture was stirred at 25° C. for 1 hr under N₂ atmosphere. LC-MS showed 5 was consumed completely and one main peak with desired mass was detected. The residue mixture was diluted with H₂O (30 mL) and extracted with EtOAc (20 mL*3). The combined organic layers were washed with brine (20 mL), dried over Na₂SO₄, filtered and concentrated under reduced pressure to give a residue. The residue was purified by prep-TLC (SiO₂, Petroleum ether/Ethyl acetate=10/1, Product R_{f}=0.51). 6 (390 mg, 971.89 μmol, 75.42% yield, 98.26% purity) was obtained as a white solid.

LCMS: RT=1.466 min, MS cal.: 394.10, [M+H]⁺=294.9

¹H NMR (400 MHz, CHLOROFORM-d) δ=7.98 (br d, J=11.6 Hz, 2H), 4.00 (s, 3H), 3.25 (s, 3H), 1.45-1.30 (m, 9H)

### General Procedure for Preparation of Intermediate 7:

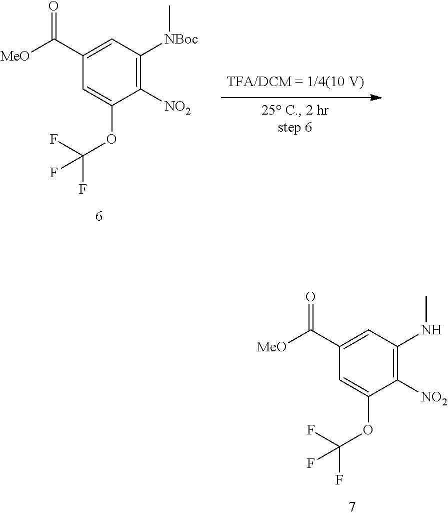

A mixture of 6 (320 mg, 811.57 μmol, 1 eq) in TFA (0.8 mL) and DCM (3.2 mL) was degassed and purged with N₂ for 3 times, and then the mixture was stirred at 25° C. for 2 hrs under N₂ atmosphere. TLC indicated Reactant 1 was consumed completely and one new spot formed. The reaction was clean according to TLC (TLC-Petroleum ether/Ethyl acetate=10/1, Product R_{f}=0.50). The reaction mixture was concentrated under reduced pressure to remove solvent. 7 (238 mg, crude) was obtained as a yellow solid.

LCMS: RT=1.302 min, MS cal.: 294.05, [M+H]⁺=294.9

¹H NMR (400 MHz, CHLOROFORM-d₄) δ=7.45 (s, 1H), 7.25 (s, 1H), 6.29 (br dd, J=2.0, 3.6 Hz, 1H), 3.97 (s, 3H), 3.03 (br s, 3H)

### General Procedure for Preparation of Intermediate 8:

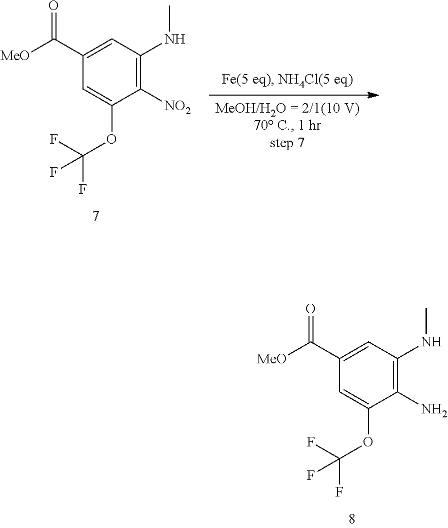

A mixture of 7 (238 mg, 809.02 μmol, 1 eq) in MeOH (2 mL) was added NH₄Cl (216.38 mg, 4.05 mmol, 5 eq) in H₂O (1 mL), degassed and purged with N₂ for 3 times, and then Fe (225.90 mg, 4.05 mmol, 5 eq) was added to the reaction mixture. After addition, the mixture was stirred at 70° C. for 1 hr under N₂ atmosphere. LC-MS showed 7 was consumed completely and one main peak with desired mass was detected. The reaction mixture was filtered and concentrated under reduced pressure to give a residue. The residue was diluted with H₂O (40 mL) and extracted with EtOAc (40 mL*3). The combined organic layers were washed with brine (30 mL), dried over Na₂SO₄, filtered and concentrated under reduced pressure to give a residue. 8 (200 mg, crude) was obtained as a white solid.

LCMS: RT=1.118 min, MS cal.: 264.07, [M+H]⁺=264.9

¹H NMR (400 MHz, CHLOROFORM-d) δ=7.48 (s, 1H), 7.32 (s, 1H), 3.89 (s, 3H), 2.94 (s, 3H)

### General Procedure for Preparation of Intermediate 9:

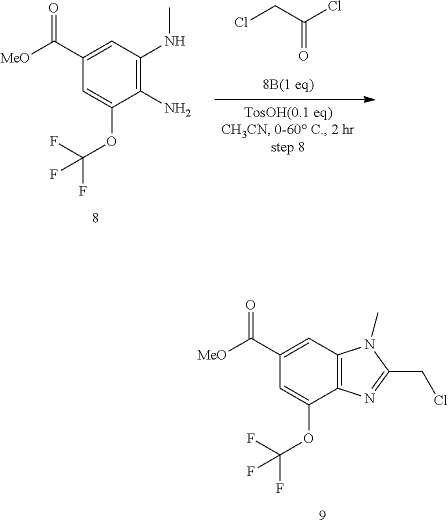

A mixture of 8 (100 mg, 378.50 μmol, 1 eq), TosOH (6.52 mg, 37.85 μmol, 0.1 eq) in ACN (1 mL) was degassed and purged with N₂ for 3 times, and then Cooled to 0° C. 8A (42.75 mg, 378.50 μmol, 30.15 μL, 1 eq) in ACN (0.5 mL) was added to the reaction mixture at 0° C. and then the mixture was stirred at 60° C. for 2 hr under N₂ atmosphere. LC-MS showed 8 was consumed completely and one main peak with desired mass was detected. The residue mixture was concentrated under reduced pressure to give a residue. 9 (122 mg, crude) was obtained as a white solid.

LCMS: RT=1.147 min, MS cal.: 322.03, [M+H]⁺=323.0

### General Procedure for Preparation of Intermediate 10:

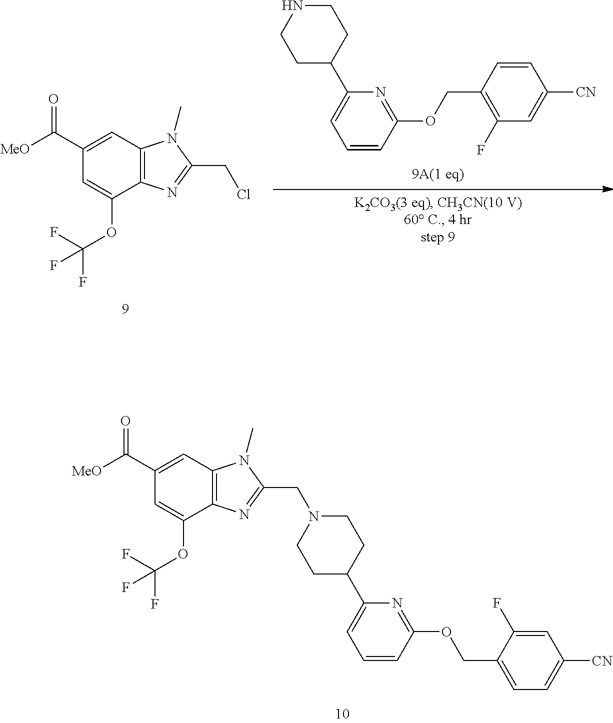 [SMILES: COC(=O)c1cc(OC(F)(F)F)c2nc(CCl)n(C)c2c1 ; N#Cc1ccc(COc2cccc(C3CCNCC3)n2)c(F)c1 ; "9A(1 eq)" ; "K2CO3(3 eq), CH3CN(10 V)60° C., 4 hrstep 9" ; COC(=O)c1cc(OC(F)(F)F)c2nc(CN3CCC(c4cccc(OCc5ccc(C#N)cc5F)n4)CC3)n(C)c2c1]

A mixture of 9 (122 mg, 378.10 μmol, 1 eq), 9A (117.72 mg, 378.10 μmol, 1 eq), K₂CO₃ (156.77 mg, 1.13 mmol, 3 eq) in ACN (2 mL) was degassed and purged with N₂ for 3 times, and then the mixture was stirred at 60° C. for 4 hrs under N₂ atmosphere. LC-MS showed 9 was consumed completely and one main peak with desired mass was detected. The residue mixture was diluted with H₂O (20 mL) and extracted with EtOAc (20 mL*3). The combined organic layers were washed with brine (20 mL), dried over Na₂SO₄, filtered and concentrated under reduced pressure to give a residue. The residue was purified by column chromatography (SiO₂, Petroleum ether/Ethyl acetate=100/1 to 3/1, TLC-Petroleum ether/Ethyl acetate=2/1, Product R_{f}=0.42). 10 (180 mg, 281.37 μmol, 74.42% yield, 93.41% purity) was obtained as a white solid.

LCMS: RT=1.604 min, MS cal.: 597.20, [M+H]⁺=598.0

¹H NMR (400 MHz, CHLOROFORM-d₄) δ=8.09 (d, J=1.2 Hz, 1H), 7.87-7.84 (m, 1H), 7.63 (t, J=7.2 Hz, 1H), 7.53 (t, J=7.6 Hz, 1H), 7.44 (dd, J=1.2, 7.9 Hz, 1H), 7.37 (dd, J=1.2, 9.3 Hz, 1H), 6.76 (d, J=7.2 Hz, 1H), 6.65 (d, J=8.0 Hz, 1H), 5.50 (s, 2H), 4.03 (s, 3H), 3.98 (s, 4H), 3.95 (br s, 2H), 2.97 (br s, 2H), 2.62 (br s, 1H), 2.34 (br s, 2H), 1.92-1.75 (m, 4H)

### General Procedure for Preparation of Compound 125 (11):

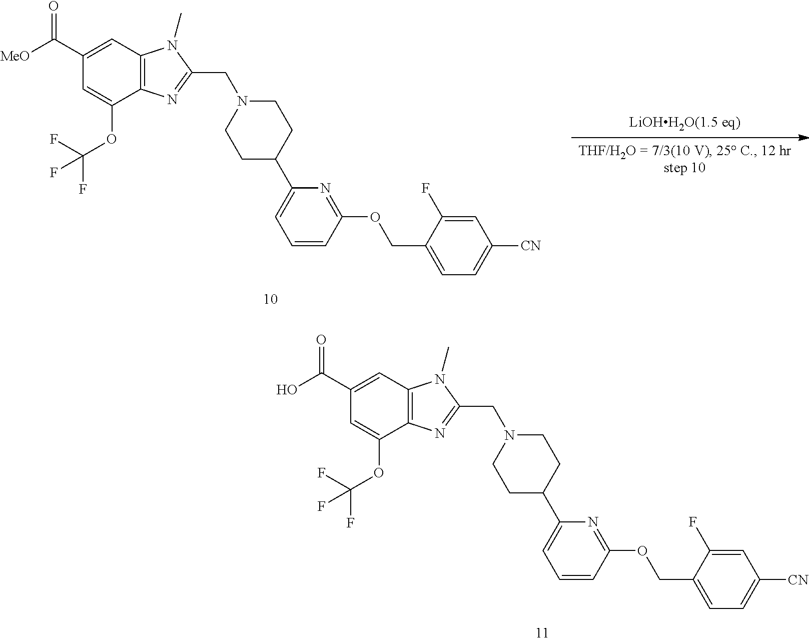

A mixture of 10 (70 mg, 117.14 μmol, 1 eq), LiOH·H₂O (7.37 mg, 175.71 μmol, 1.5 eq) in THF (0.7 mL) and H₂O (0.3 mL) was degassed and purged with N₂ for 3 times, and then the mixture was stirred at 25° C. for 12 hrs under N₂ atmosphere. LC-MS (ET62904-564-p1a1) showed 10 was consumed completely and one main peak with desired mass was detected. The reaction mixture was diluted with ACN 0.5 mL. The liquid was purified by prep-HPLC (column: Waters Xbridge Prep OBD C₁₈ 150*40 mm*10 um; mobile phase: [H₂O (10 mM NH₄HCO₃)-ACN]; gradient: 15%-55% B over 8.0 min). 2-((4-(6-((4-Cyano-2-fluorobenzyl)oxy)pyridin-2-yl)piperidin-1-yl)methyl)-1-methyl-4-(trifluoromethoxy)-1H-benzo[d]imidazole-6-carboxylic acid (29.24 mg, 49.63 μmol, 42.37% yield, 99.05% purity) was obtained as a white solid.

LCMS: RT=1.098 min, MS cal.: 583.18, [M+H]⁺=584.0

¹H NMR (400 MHz, METHANOL-d₄) δ=8.24 (d, J=1.2 Hz, 1H), 7.85 (s, 1H), 7.67 (t, J=7.6 Hz, 1H), 7.62-7.50 (m, 3H), 6.84 (d, J=7.2 Hz, 1H), 6.68 (d, J=8.0 Hz, 1H), 5.50 (s, 2H), 4.04 (s, 3H), 4.00 (s, 2H), 3.08 (br d, J=11.2 Hz, 2H), 2.66 (td, J=7.6, 15.5 Hz, 1H), 2.45-2.36 (m, 2H), 1.89-1.81 (m, 4H)

### Example A210—Preparation of Compound 123 and Compound 124. (S)-2-((4-(3-(4-Cyano-2-fluorophenyl)-2,3-dihydrobenzo[b][1,4]dioxin-5-yl)piperidin-1-yl)methyl)-4-(difluoromethoxy)-1-methyl-1H-benzo[d]imidazole-6-carboxylic acid (Compound 123) (3A) & (R)-2-((4-(3-(4-Cyano-2-fluorophenyl)-2,3-dihydrobenzo[b][1,4]dioxin-5-yl)piperidin-1-yl)methyl)-4-(difluoromethoxy)-1-methyl-1H-benzo[d]imidazole-6-carboxylic acid (Compound 124) (3B)

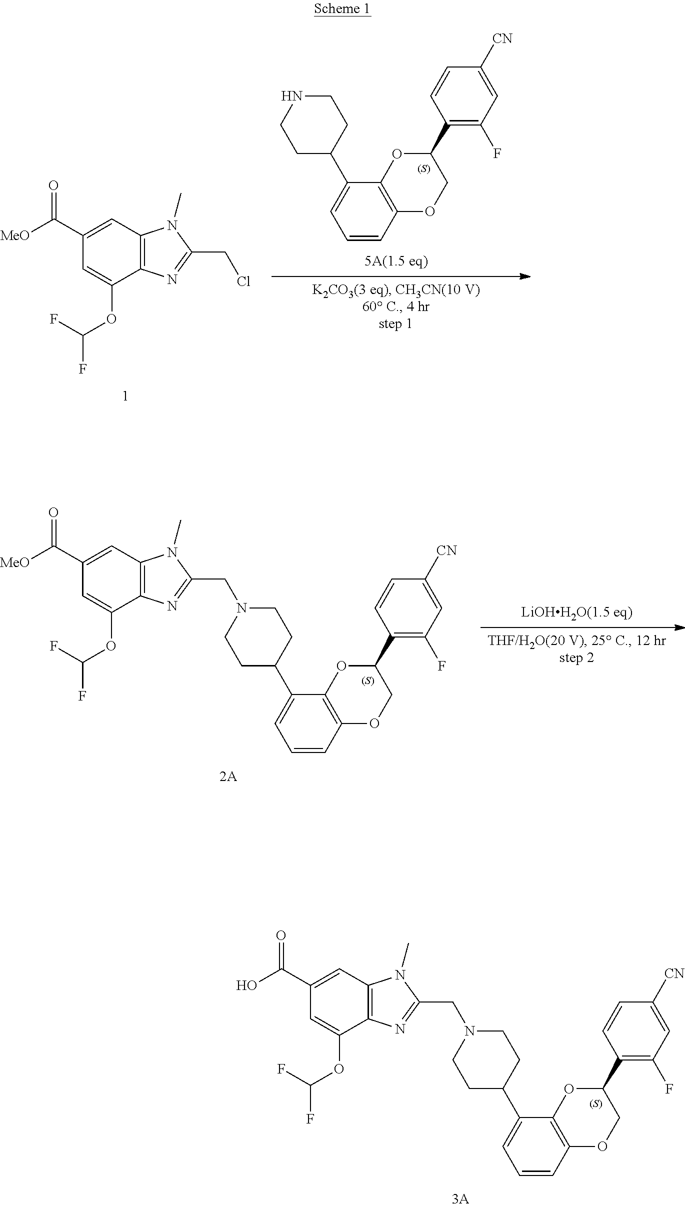

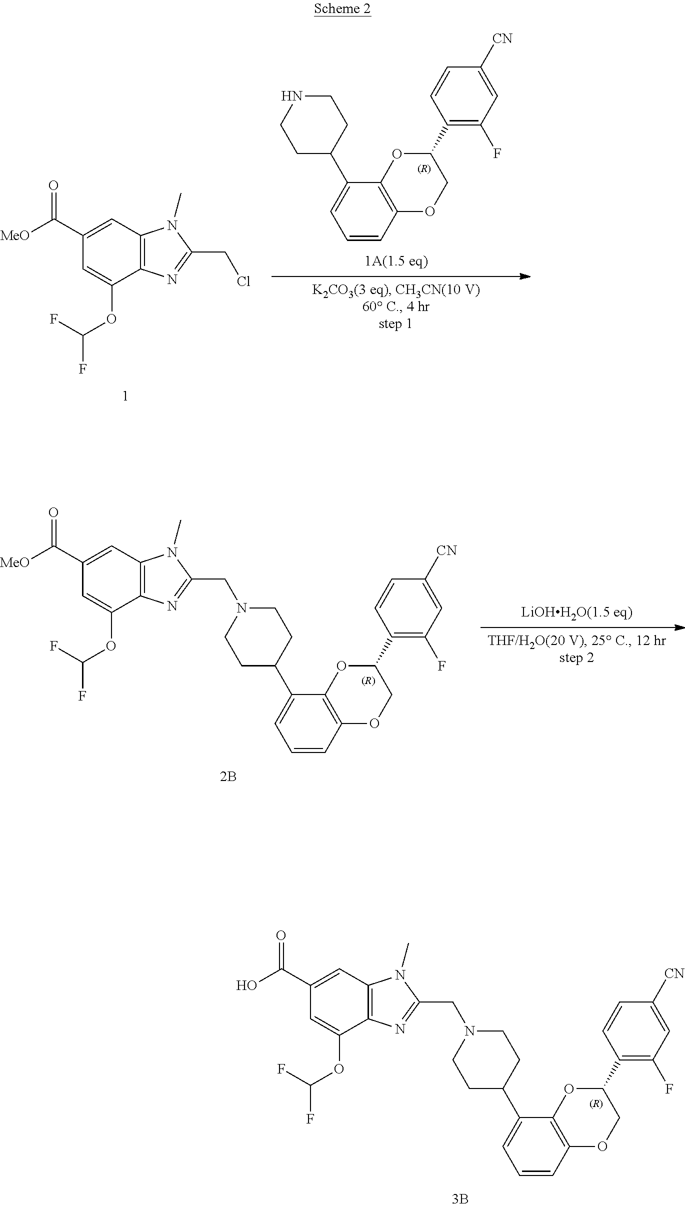

### General Procedure for Preparation of Scheme 1, Intermediate 2:

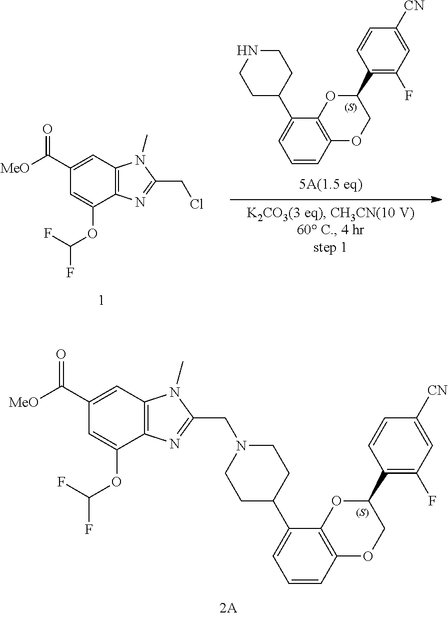

To a solution of 1 (50 mg, 164.11 μmol, 1 eq) and 5A (73.82 mg, 196.93 μmol, 1.2 eq, HCl) in

CH₃CN (0.5 mL) was added K₂CO₃ (68.04 mg, 492.33 μmol, 3 eq). The mixture was stirred at 60° C. for 1 hr. LC-MS showed Reactant 1 was consumed completely. The mixture was filtered and concentrated in vacuum. The residue was purified by column chromatography (SiO₂, Petroleum ether/Ethyl acetate=1/0 to 3/1). 2A (71.5 mg, 117.87 μmol, 71.83% yield) was a white solid.

¹H NMR (400 MHz, DMSO-d6) δ=8.10 (d, J=1.2 Hz, 1H), 7.99 (dd, J=1.2, 10.4 Hz, 1H), 7.85-7.80 (m, 1H), 7.71-7.62 (m, 2H), 7.53 (s, 1H), 6.87-6.76 (m, 3H), 5.58 (dd, J=2.0, 7.6 Hz, 1H), 4.48 (dd, J=2.0, 11.6 Hz, 1H), 4.11 (dd, J=7.6, 11.6 Hz, 1H), 3.95 (s, 3H), 3.90 (s, 3H), 3.84 (s, 2H), 2.99-2.82 (m, 3H), 2.27-2.11 (m, 2H), 1.80 (br d, J=11.2 Hz, 1H), 1.74-1.56 (m, 3H

### General Procedure for Preparation of Compound 123 (3A):

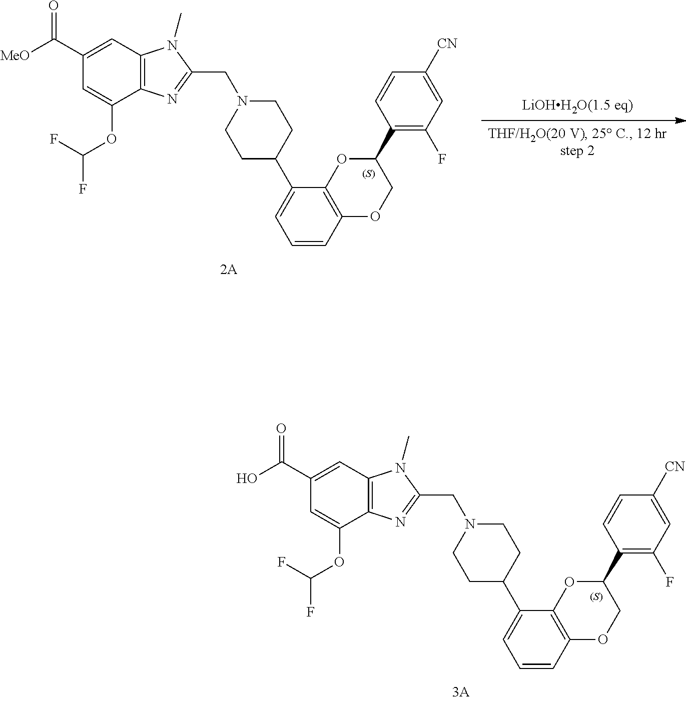

To a solution of 2A (50 mg, 82.43 μmol, 1 eq) in H₂O (0.15 mL) and THF (0.35 mL) was added LiOH·H₂O (5.19 mg, 123.64 μmol, 1.5 eq). The mixture was stirred at 25° C. for 12 hr. LCMS indicated Reactant 1 was consumed completely and one main peak with desired mass was detected. The residue was purified by prep-HPLC (column: Waters Xbridge Prep OBD C18 150*40 mm*10 um; mobile phase: [H₂O (10 mM NH₄HCO₃)-ACN]; gradient: 20%-70% B over 8.0 min) to give product. (S)-2-((4-(3-(4-Cyano-2-fluorophenyl)-2,3-dihydrobenzo[b][1,4]dioxin-5-yl)piperidin-1-yl)methyl)-4-(difluoromethoxy)-1-methyl-1H-benzo[d]imidazole-6-carboxylic acid (27.71 mg, 46.77 μmol, 56.75% yield) was a white solid.

LCMS: RT=2.690 min, MS cal.: 592.1, [M+H]⁺=593.2

HPLC: RT=11.120 min, purity: 99.06%

¹H NMR (400 MHz, DMSO-d₆) δ=8.06 (d, J=1.0 Hz, 1H) 8.00 (d, J=10.4 Hz, 1H) 7.84 (dd, J=8.0, 1.2 Hz, 1H) 7.67 (t, J=7.6 Hz, 1H) 7.62 (t, J=74.4 Hz, 1H)) 7.51 (s, 1H) 6.85-6.81 (m, 2H) 6.81-6.75 (m, 1H) 5.59 (dd, J=7.2, 2.0 Hz, 1H) 4.51-4.44 (m, 1H) 4.16-4.07 (m, 1H) 3.94 (s, 3H) 3.83 (s, 2H) 2.93 (br d, J=11.2 Hz, 2H) 2.90-2.81 (m, 1H) 2.24-2.13 (m, 2H) 1.78 (br d, J=12.0 Hz, 1H) 1.70-1.64 (m, 3H)

### General Procedure for Preparation of Intermediate 2B:

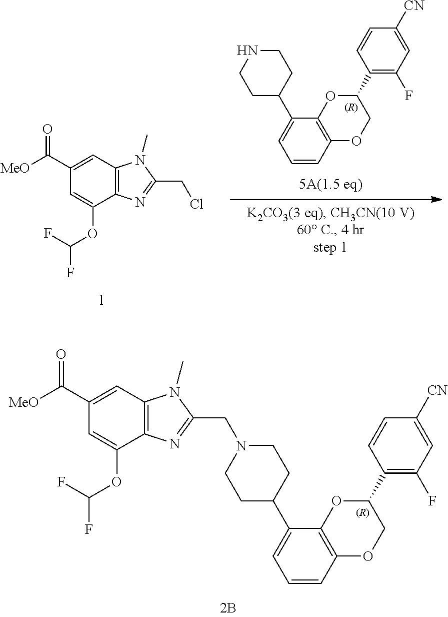

A mixture of 1 (94.54 mg, 310.31 μmol, 1 eq) in CH₃CN (1 mL) was added 1A (105 mg, 310.31 μmol, 1 eq) and K₂CO₃ (128.66 mg, 930.92 μmol, 3 eq). The mixture was stirred at 60° C. for 1 hr. LC-MS showed 1 was consumed completely and desired mass was detected. The reaction mixture was diluted with H₂O (10 mL) and extracted with EtOAc (5 mL*3). The combined organic layers were washed with brine (60 mL), dried over Na₂SO₄, filtered, and concentrated under reduced pressure to give a residue. The residue was purified by column chromatography (SiO₂, Petroleum ether/Ethyl acetate=3/1 to 1/1) to give 2B (160 mg, 263.77 μmol, 85.00% yield) was obtained as a white solid.

LCMS: RT=0.473 min, MS cal.: 606.2, [M+H]⁺=607.3

¹H NMR (400 MHz, DMSO-d₆) δ=8.10 (d, J=1.08 Hz, 1H) 7.99 (d, J=10.12 Hz, 1H) 7.84 (s, 1H) 7.82 (s, 1H) 7.70-7.65 (m, 1H) 7.63 (t, J=72.8 Hz, 1H) 7.53 (s, 1H) 6.86-6.81 (m, 2H) 6.80-6.74 (m, 1H) 5.58 (br d, J=5.96 Hz, 1H) 4.51-4.43 (m, 1H) 4.11 (br dd, J=11.64, 7.69 Hz, 1H) 3.98-3.94 (m, 3H) 3.90 (s, 3H) 3.87-3.80 (m, 2H) 2.98-2.89 (m, 2H) 2.88-2.77 (m, 1H) 2.25-2.19 (m, 2H) 1.84-1.75 (m, 1H) 1.73-1.47 (m, 3H)

### General Procedure for Preparation of Compound 124 (3B):

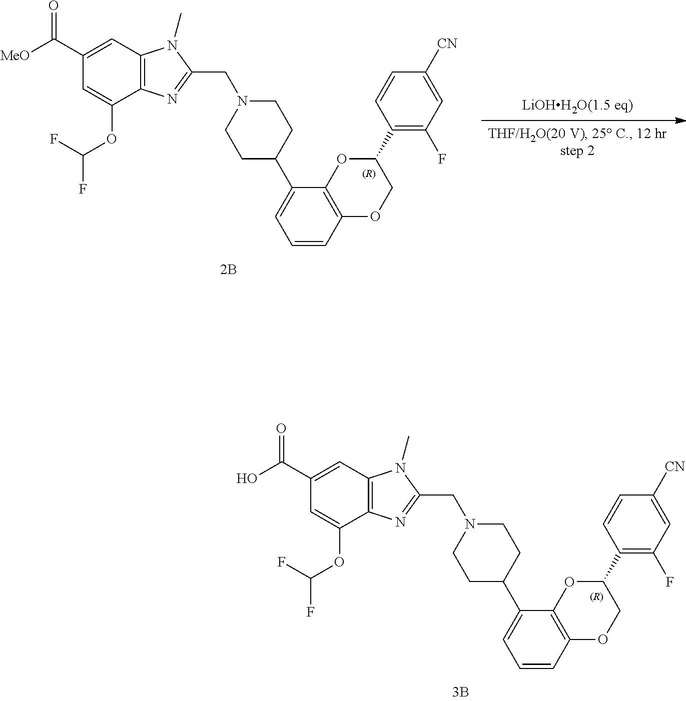

A mixture of 2B (60 mg, 98.91 μmol, 1 eq), LiOH H₂O (6.23 mg, 148.37 μmol, 1.5 eq) in THF (0.7 mL) and H₂O (0.3 mL) was degassed and purged with N₂ for 3 times, and then the mixture was stirred at 25° C. for 16 hr under N₂ atmosphere. LCMS (ET43536-1481-P1A1) showed the reaction was consumed completely. The residue was purified by prep-HPLC (neutral condition; column: Waters Xbridge Prep OBD C18 150*40 mm*10 um; mobile phase: [H₂O (10 mM NH₄HCO₃)-ACN]; gradient: 15%-55% B over 8.0 min). (R)-2-((4-(3-(4-Cyano-2-fluorophenyl)-2,3-dihydrobenzo[b][1,4]dioxin-5-yl)piperidin-1-yl)methyl)-4-(difluoromethoxy)-1-methyl-1H-benzo[d]imidazole-6-carboxylic acid (28.53 mg, 47.97 μmol, 48.50% yield, 99.63% purity) was obtained as a white solid.

LCMS: RT=2.405 min, MS cal.: 592.1, [M+H]⁺=593.2

HPLC: RT=11.090 min, purity: 99.63%

¹H NMR (400 MHz, DMSO-d₆) δ=8.06 (d, J=0.92 Hz, 1H) 8.00 (d, J=10.36 Hz, 1H) 7.84 (dd, J=7.92, 1.13 Hz, 1H) 7.67 (t, J=7.68 Hz, 1H) 7.62 (t, J=74.4 Hz, 1H) 7.51 (s, 1H) 6.85-6.81 (m, 2H) 6.81-6.75 (m, 1H) 5.59 (dd, J=7.32, 1.85 Hz, 1H) 4.51-4.44 (m, 1H) 4.16-4.07 (m, 1H) 3.94 (s, 3H) 3.83 (s, 2H) 2.93 (br d, J=11.32 Hz, 2H) 2.90-2.81 (m, 1H) 2.24-2.13 (m, 2H) 1.80 (br d, J=11.92 Hz, 1H) 1.74-1.66 (m, 2H) 1.65-1.54 (m, 1H)

### Example A211—Preparation of Compound 121. 2-((4-(6-((4-Cyano-2-fluorobenzyl)oxy)pyridin-2-yl)piperidin-1-yl)methyl)-1-methyl-4-(2,2,2-trifluoroethoxy)-1H-benzo[d]imidazole-6-carboxylic acid (Compound 121) (3)

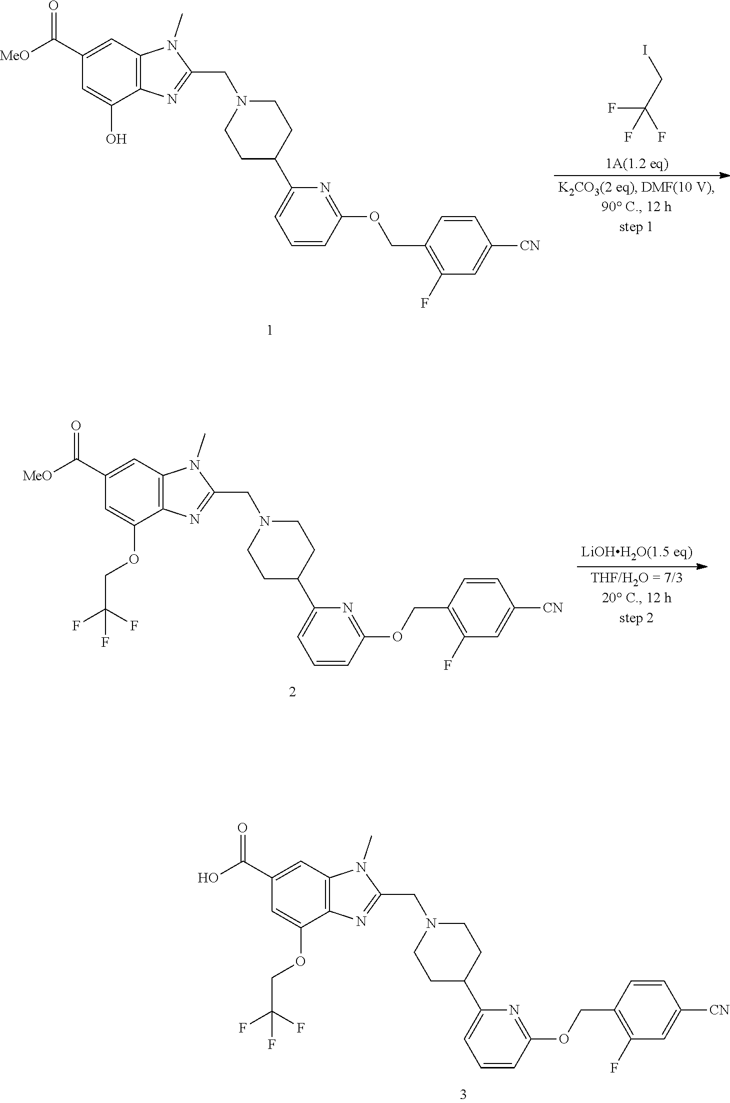 [SMILES: COC(=O)c1cc(O)c2nc(CN3CCC(c4cccc(OCc5ccc(C#N)cc5F)n4)CC3)n(C)c2c1 ; FC(F)(F)CI ; COC(=O)c1cc(OCC(F)(F)F)c2nc(CN3CCC(c4cccc(OCc5ccc(C#N)cc5F)n4)CC3)n(C)c2c1 ; Cn1c(CN2CCC(c3cccc(OCc4ccc(C#N)cc4F)n3)CC2)nc2c(OCC(F)(F)F)cc(C(=O)O)cc21]

### General Procedure for Preparation of Intermediate 2:

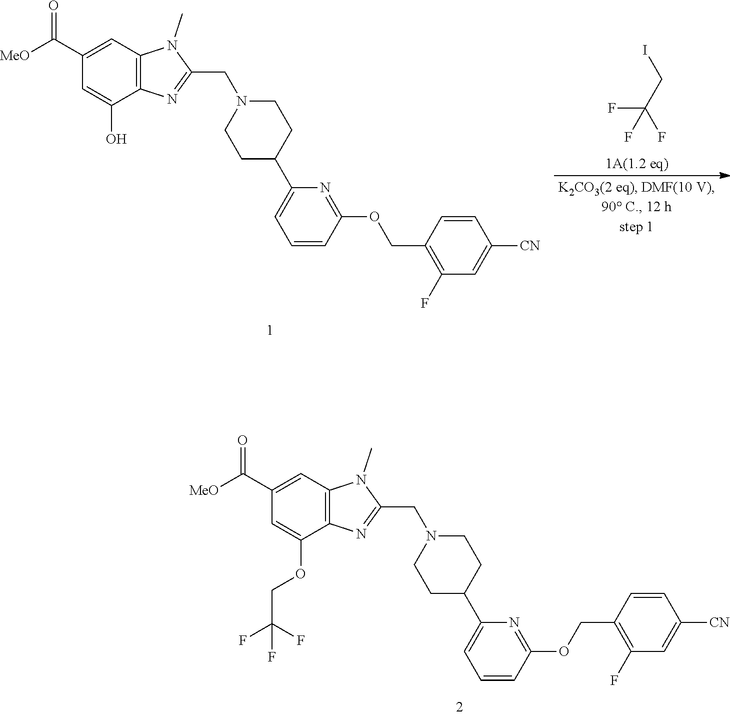 [SMILES: COC(=O)c1cc(O)c2nc(CN3CCC(c4cccc(OCc5ccc(C#N)cc5F)n4)CC3)n(C)c2c1 ; FC(F)(F)CI ; COC(=O)c1cc(OCC(F)(F)F)c2nc(CN3CCC(c4cccc(OCc5ccc(C#N)cc5F)n4)CC3)n(C)c2c1]

A mixture of 1 (90 mg, 169.95 μmol, 1 eq), 1A (42.81 mg, 203.94 μmol, 20.01 μL, 1.2 eq), K₂CO₃ (46.98 mg, 339.90 μmol, 2 eq) in DMF (1 mL) was degassed and purged with N₂ for 3 times, and then the mixture was stirred at 90° C. for 12 hr under N₂ atmosphere. LCMS showed 1 was consumed completely and one main peak with desired mass was detected. The reaction mixture was diluted with H₂O (5 mL) and extracted with EtOAc (5 mL*3). The combined organic layers were washed with brine (5 mL*3), dried over Na₂SO₄, filtered, and concentrated under reduced pressure to give a residue. The residue was purified by column chromatography (SiO₂, Petroleum ether/Ethyl acetate=10/1 to 0/1). 2 (60 mg, 98.11 μmol, 57.73% yield) was obtained as yellow oil.

LCMS: RT=0.467 min, MS cal.: 611.2, [M+H]⁺=612.1

### General Procedure for Preparation of Compound 121 (3):

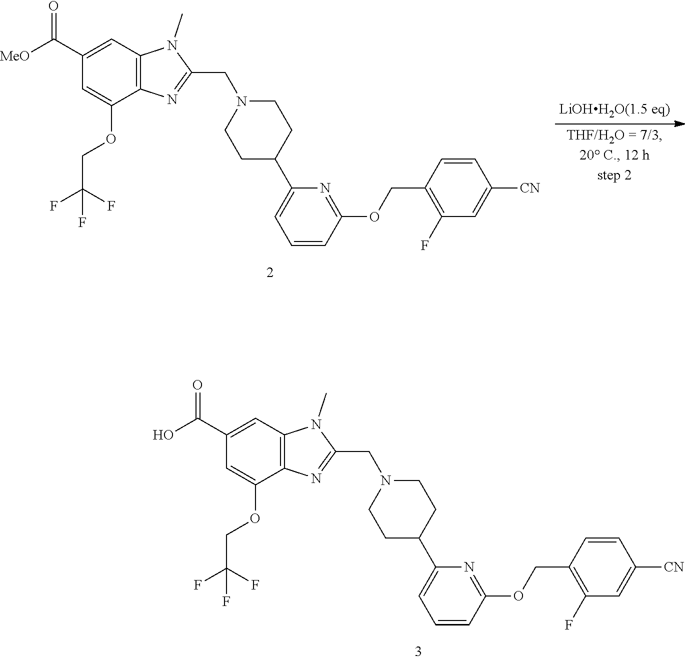 [SMILES: COC(=O)c1cc(OCC(F)(F)F)c2nc(CN3CCC(c4cccc(OCc5ccc(C#N)cc5F)n4)CC3)n(C)c2c1 ; Cn1c(CN2CCC(c3cccc(OCc4ccc(C#N)cc4F)n3)CC2)nc2c(OCC(F)(F)F)cc(C(=O)O)cc21]

A mixture of 2 (60 mg, 98.11 μmol, 1 eq), LiOH·H₂O (6.18 mg, 147.16 μmol, 1.5 eq) in THF (0.7 mL) and H₂O (0.3 mL) was degassed and purged with N₂ for 3 times, and then the mixture was stirred at 25° C. for 12 hr under N₂ atmosphere. LCMS showed 2 was consumed completely and one main peak with desired mass was detected. The reaction mixture was added with 0.5M citric acid to adjust pH=7. The mixture was purified by prep-HPLC (column: Waters Xbridge Prep OBD C18 150*40 mm*10 um; mobile phase: [H₂O (10 mM NH₄HCO₃)-ACN]; gradient: 25%-75% B over 8.0 min). 2-((4-(6-((4-Cyano-2-fluorobenzyl)oxy)pyridin-2-yl)piperidin-1-yl)methyl)-1-methyl-4-(2,2,2-trifluoroethoxy)-1H-benzo[d]imidazole-6-carboxylic acid (27.61 mg, 46.20 μmol, 47.10% yield) was obtained as a white solid.

LCMS: RT=2.711 min, MS cal.: 597.2, [M+H]⁺=598.2

HPLC: RT=11.089 min, purity: 90.84%

¹H NMR (400 MHz, DMSO-d₆) δ=7.91-7.85 (m, 2H), 7.69 (d, J=3.6 Hz, 2H), 7.64 (t, J=7.6 Hz, 1H), 7.37 (d, J=0.8 Hz, 1H), 6.88 (d, J=7.2 Hz, 1H), 6.71 (d, J=8.4 Hz, 1H), 5.45 (s, 2H), 5.10 (q, J=8.8 Hz, 2H), 3.94 (s, 3H), 3.83 (s, 2H), 2.91 (br d, J=11.2 Hz, 2H), 2.57 (br s, 1H), 2.19 (br t, J=10.8 Hz, 2H), 1.79-1.59 (m, 4H)

### Example A212—Preparation of Compound 120. 2-(4-(6-((4-Cyano-2-fluorobenzyl)oxy)pyridin-2-yl)-2-fluorobenzyl)-4-(difluoromethoxy)-1-methyl-1H-benzo[d]imidazole-6-carboxylic acid (Compound 120) (5)

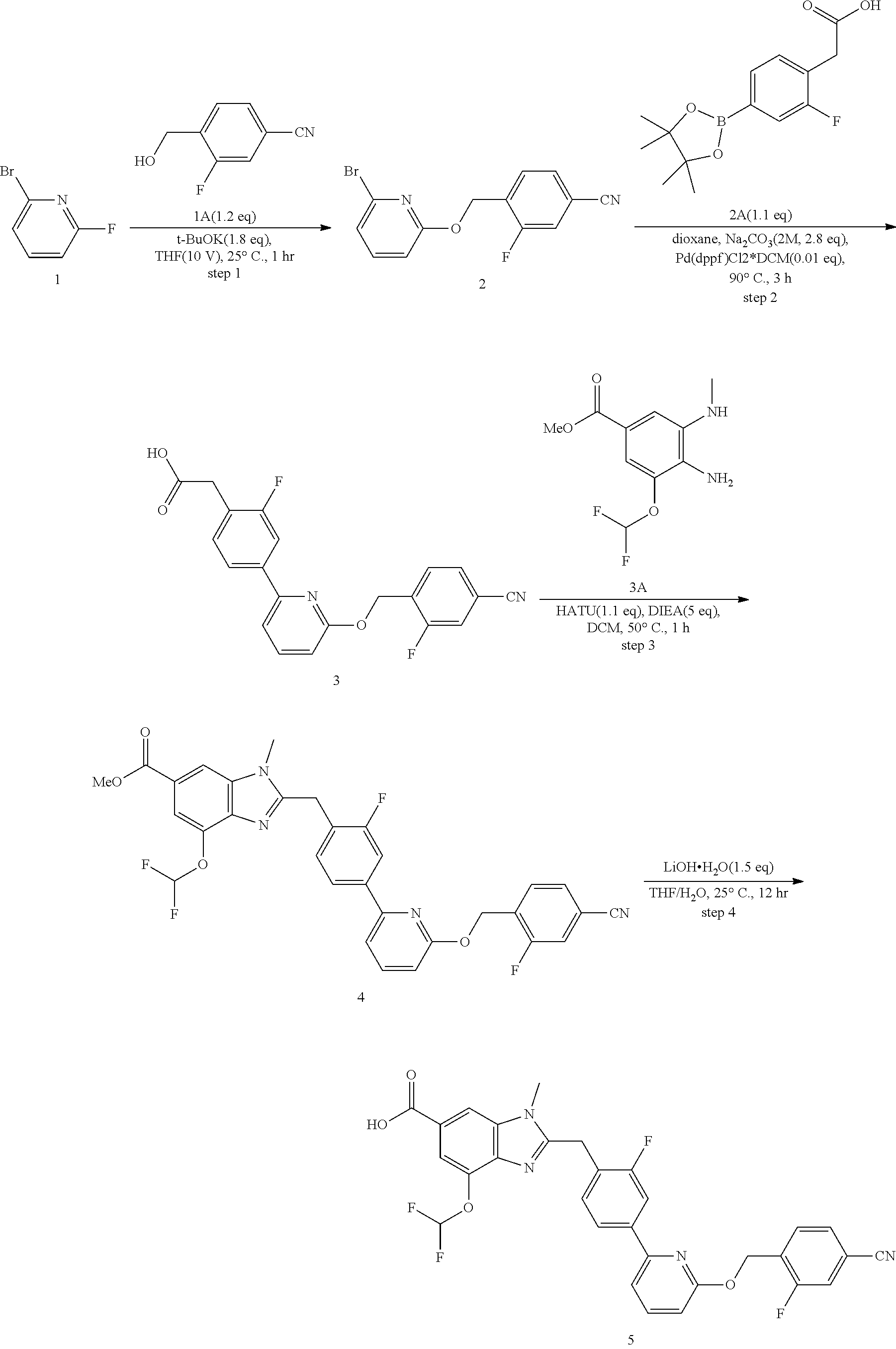

### General Procedure for Preparation of Intermediate 2:

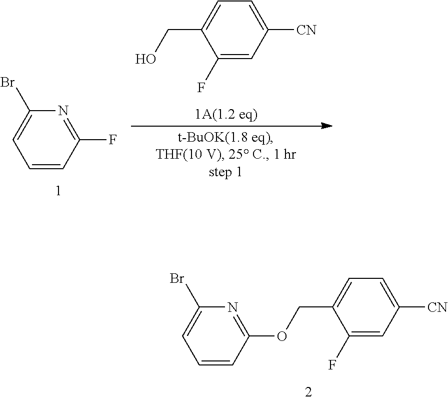 [SMILES: Fc1cccc(Br)n1 ; N#Cc1ccc(CO)c(F)c1 ; N#Cc1ccc(COc2cccc(Br)n2)c(F)c1]

To a solution of 1 (5 g, 28.41 mmol, 1 eq) in THF (50 mL) was added t-BuOK (1 M, 51.14 mL, 1.8 eq) and 1A (5.15 g, 34.09 mmol, 1.2 eq). The mixture was stirred at 25° C. for 1 hr. LC-MS showed 1 was consumed completely and desired mass was detected. The reaction mixture was quenched by NH₄Cl aq (50 mL), then diluted with H₂O (20 mL) and extracted with EtOAc (20 mL*2). The combined organic layers were washed with brine (15 mL*2), dried over Na₂SO₄, filtered and concentrated under reduced pressure to give a residue. The residue was purified by column chromatography (SiO₂, Petroleum ether/EtOAc=50/1 to 2/1). 2 (2.9 g, 9.44 mmol, 33.24% yield) was obtained as a white solid.

LCMS: RT=0.830 min, MS cal.: 306.0, 308.0, [M+H]⁺=307.1

¹H NMR (400 MHz, CHCl3-d) δ=7.67 (t, J=7.6 Hz, 1H), 7.52-7.44 (m, 2H), 7.40 (d, J=9.2 Hz, 1H), 7.13 (d, J=7.6 Hz, 1H), 6.78 (d, J=8.4 Hz, 1H), 5.49 (s, 2H)

### General Procedure for Preparation of Intermediate 3:

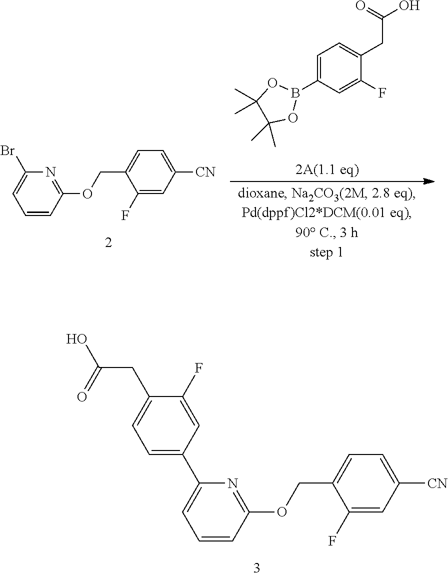 [SMILES: CC1(C)OB(c2ccc(CC(=O)O)c(F)c2)OC1(C)C ; N#Cc1ccc(COc2cccc(Br)n2)c(F)c1 ; N#Cc1ccc(COc2cccc(-c3ccc(CC(=O)O)c(F)c3)n2)c(F)c1]

A mixture of 2 (1 g, 3.26 mmol, 1 eq), 2A (1.00 g, 3.58 mmol, 1.1 eq), Na₂CO₃ (2 M, 4.56 mL, 2.8 eq), in dioxane (15.6 mL) was sparged with N₂ for 5 min, and Pd(dppf)Cl₂·CH₂Cl₂ (26.59 mg, 32.56 μmol, 0.01 eq) was added to the mixture. The mixture was degassed and purged with N₂ for 3 times, and then the mixture was stirred at 90° C. for 3 hr under N₂ atmosphere. LCMS indicated 2 was consumed completely and one main peak with desired mass was detected. The residue was poured into water (50 mL). The aqueous phase was extracted with EtOAc (50 mL*3). The combined organic phase was washed with brine (50 mL*2), dried with anhydrous Na₂SO₄, filtered and concentrated in vacuum. 3 (1.2 g, crude) was a white solid.

LCMS: RT=1.761 min, MS cal.: 380.1, [M+H]⁺=381.2

¹H NMR (400 MHz, DMSO-d6) δ=12.51 (br s, 1H), 7.95-7.89 (m, 1H), 7.87-7.69 (m, 5H), 7.64 (d, J=7.6 Hz, 1H), 7.44 (t, J=8.0 Hz, 1H), 6.91 (d, J=8.0 Hz, 1H), 5.61 (s, 2H), 3.68 (s, 2H)

### General Procedure for Preparation of Intermediate 4:

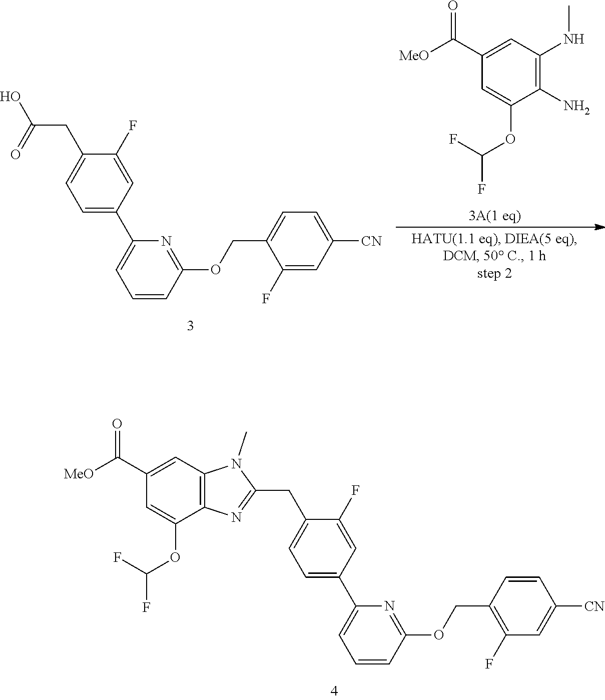 [SMILES: N#Cc1ccc(COc2cccc(-c3ccc(CC(=O)O)c(F)c3)n2)c(F)c1 ; CNc1cc(C(=O)OC)cc(OC(F)F)c1N ; COC(=O)c1cc(OC(F)F)c2nc(Cc3ccc(-c4cccc(OCc5ccc(C#N)cc5F)n4)cc3F)n(C)c2c1]

To a solution of 3 (100 mg, 406.16 μmol, 1 eq) in Py (0.5 mL) was added EDCI (194.65 mg, 1.02 mmol, 2.5 eq) and 3A (154.48 mg, 406.16 μmol, 1 eq). The mixture was stirred at 100° C. for 12 hr. LCMS indicated 3 was consumed completely and one main peak with desired mass was detected. The residue was poured into water (10 mL). The aqueous phase was extracted with EtOAc (10 mL*3). The combined organic phase was washed with brine (10 mL*2), dried with anhydrous Na₂SO₄, filtered and concentrated in vacuum. The residue was purified by column chromatography (SiO₂, Petroleum ether/EtOAc=1/0 to 1/1). 4 (150 mg, 254.01 μmol, 62.54% yield) was a white solid.

LCMS: RT=2.424 min, MS cal.: 590.16, [M+H]⁺=591.2

### General Procedure for Preparation of Compound 120 (5):

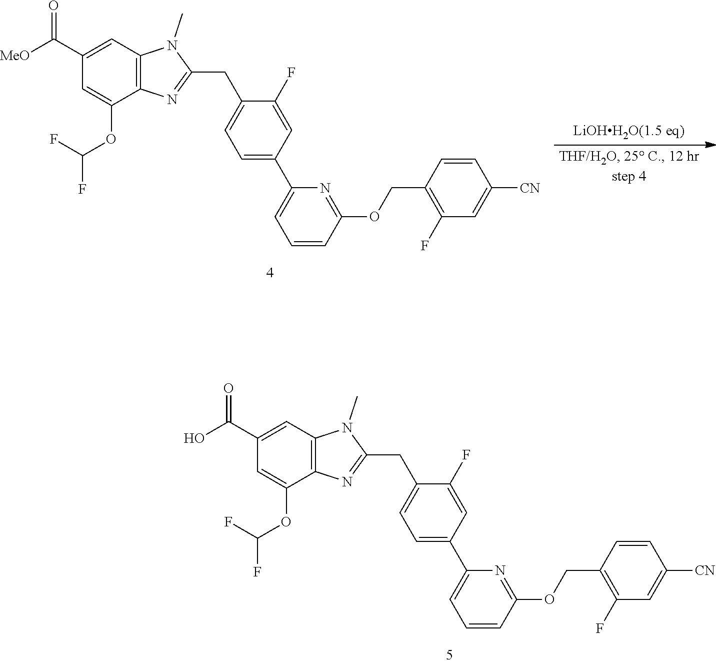 [SMILES: COC(=O)c1cc(OC(F)F)c2nc(Cc3ccc(-c4cccc(OCc5ccc(C#N)cc5F)n4)cc3F)n(C)c2c1 ; Cn1c(Cc2ccc(-c3cccc(OCc4ccc(C#N)cc4F)n3)cc2F)nc2c(OC(F)F)cc(C(=O)O)cc21]

To a solution of 4 (130 mg, 220.14 μmol, 1 eq) in THF (0.91 mL) and H₂O (0.39 mL) was added LiOH·H₂O (13.86 mg, 330.22 μmol, 1.5 eq). The mixture was stirred at 25° C. for 12 hr. LCMS indicated 4 was consumed completely and one main peak with desired mass was detected. The mixture was filtered. The residue was purified by prep-HPLC (column: Waters Xbridge Prep OBD C18 150*40 mm*10 um; mobile phase: [H₂O (10 mM NH₄HCO₃)-ACN]; gradient: 25%-55% B over 8.0 min). 2-(4-(6-((4-Cyano-2-fluorobenzyl)oxy)pyridin-2-yl)-2-fluorobenzyl)-4-(difluoromethoxy)-1-methyl-1H-benzo[d]imidazole-6-carboxylic acid (21.33 mg, 36.88 μmol, 16.75% yield, 99.679% purity) was a white solid.

LCMS: RT=2.743 min, MS cal.: 576.14, [M+H]⁺=577.1

HPLC: RT=6.994 min

¹H NMR (400 MHz, MeOH-d4) δ=8.05 (s, 1H), 7.83-7.67 (m, 5H), 7.62-7.53 (m, 2H), 7.50 (d, J=7.2 Hz, 1H), 7.25 (t, J=7.6 Hz, 1H), 7.22 (t, J=74.4 Hz, 1H), 6.85 (d, J=8.0 Hz, 1H), 5.62 (s, 2H), 4.46 (s, 2H), 3.83 (s, 3H)

### Example A213—Preparation of Compound 122. (S)-2-(1-(4-(6-((4-Cyano-2-fluorobenzyl)oxy)pyridin-2-yl)piperidin-1-yl)ethyl)-4-(difluoromethoxy)-1-methyl-1H-benzo[d]imidazole-6-carboxylic acid (Compound 119) (4A) & (R)-2-(1-(4-(6-((4-Cyano-2-fluorobenzyl)oxy)pyridin-2-yl)piperidin-1-yl)ethyl)-4-(difluoromethoxy)-1-methyl-1H-benzo[d]imidazole-6-carboxylic acid (Compound 122) (4B)

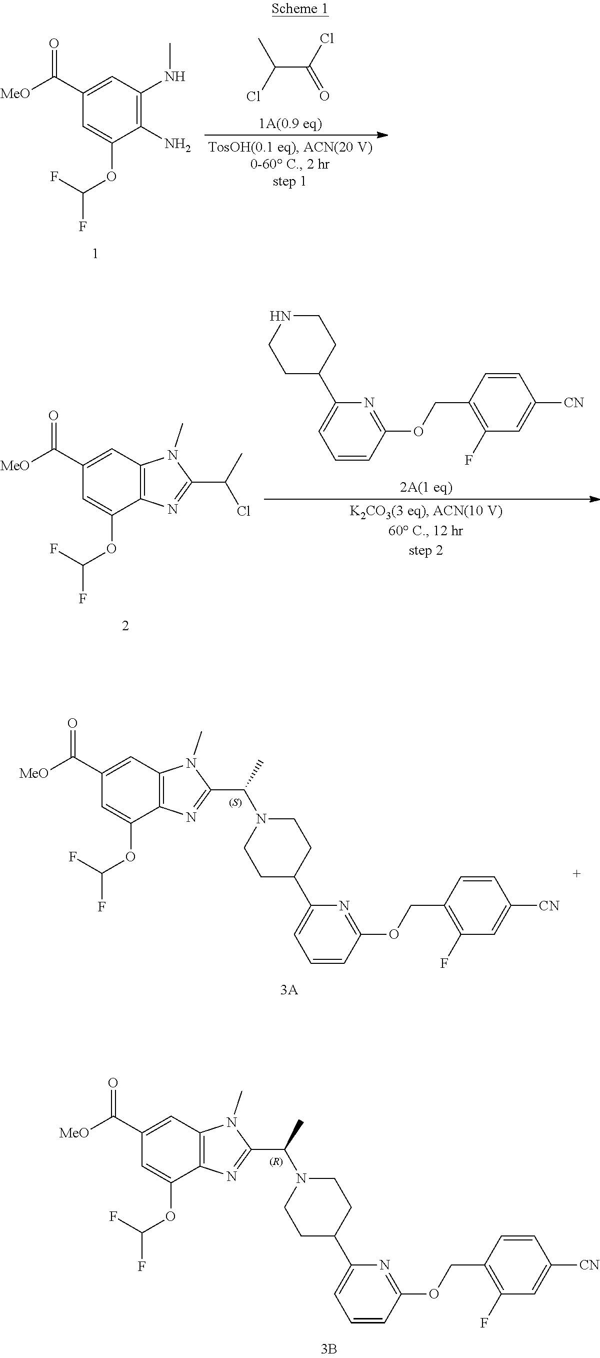

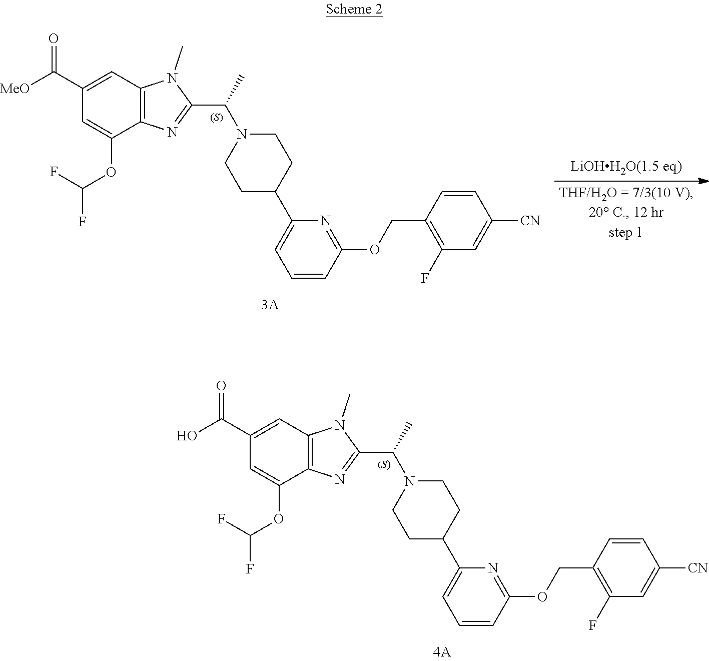

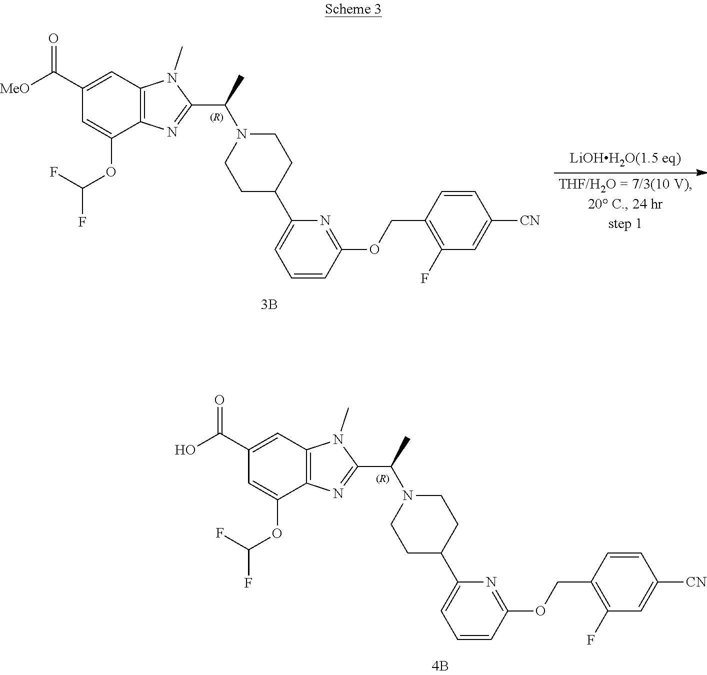

### General Procedure for Preparation of Scheme 1, Intermediate 2:

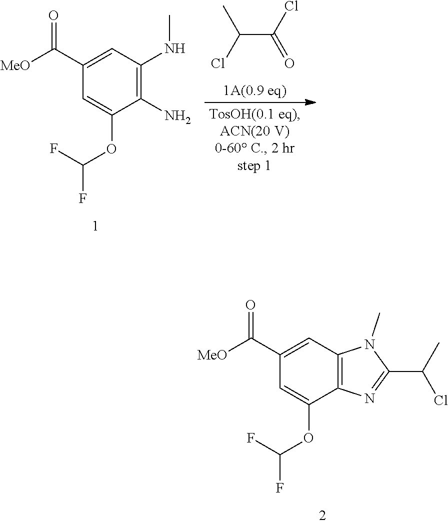

To a solution of 1 (300 mg, 1.22 mmol, 1 eq) in ACN (3 mL) was added TosOH (20.98 mg, 121.85 μmol, 0.1 eq). Then the mixture was cooled to 0° C. and 1A (139.24 mg, 1.10 mmol, 106.45 μL, 0.9 eq) was added. The mixture was stirred at 60° C. for 2 hr. TLC (Petroleum ether/Ethyl acetate=3/1, R_{f}=0.55) indicated 1 was consumed completely and two new spots formed. The reaction was clean according to TLC. The reaction mixture was partitioned between H₂O 20 mL and EtOAc 60 mL. The organic phase was separated, washed with brine 20 mL, dried over Na₂SO₄, filtered and the filtrate was concentrated under reduced pressure to give a residue. The residue was purified by column chromatography (SiO₂, Petroleum ether/Ethyl acetate=20/1 to 5/1). 2 (280 mg, 878.56 μmol, 72% yield) was obtained as a white solid.

LCMS: RT=1.583 min, MS cal.: 318.1, 320.1, [M+H]⁺=319.1, 321.1

¹H NMR (400 MHz, CHCl3-d) δ=7.97 (s, 1H), 7.72 (s, 1H), 7.47 (t, J=74.8 Hz, 1H), 5.31 (q, J=6.8 Hz, 1H), 3.97 (s, 3H), 3.94 (s, 3H), 2.14 (d, J=6.8 Hz, 3H)

### General Procedure for Preparation of Scheme 1, Intermediate 3A & 3B:

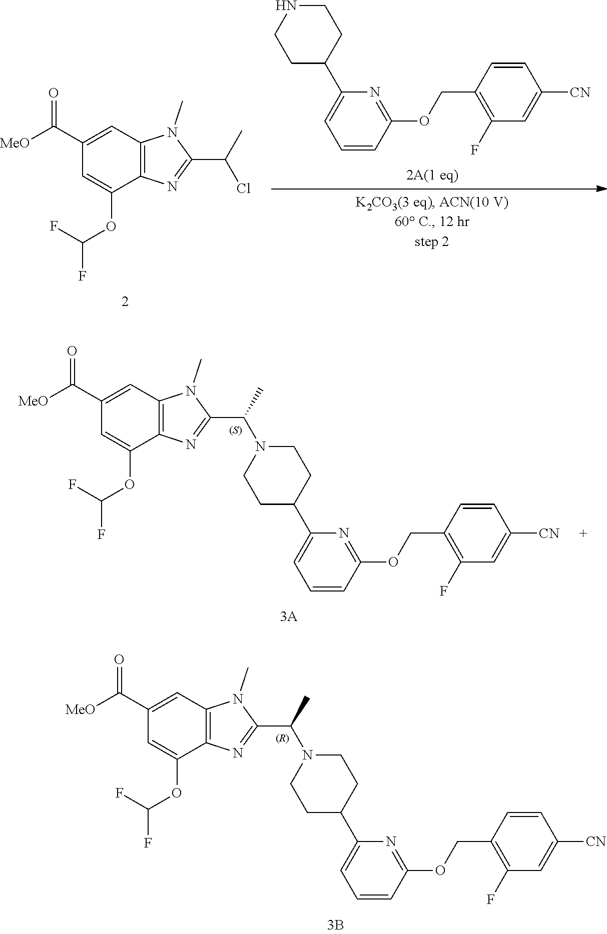

To a solution of 2 (270 mg, 847.18 μmol, 1 eq) in ACN (3 mL) was added 2A (263.77 mg, 847.18 μmol, 1 eq) and K₂CO₃ (351.26 mg, 2.54 mmol, 3 eq). The mixture was stirred at 60° C. for 12 hr. TLC (Petroleum ether/Ethyl acetate=3/1, R_{f}=0.18) indicated 2 was consumed completely and one new spot formed. The reaction was clean according to TLC. The reaction mixture was partitioned between H₂O 20 mL and EtOAc 60 mL. The organic phase was separated, washed with brine 20 mL, dried over Na₂SO₄, filtered and the filtrate concentrated under reduced pressure to give a residue. The residue was purified by column chromatography (SiO₂, Petroleum ether/Ethyl acetate=10/1 to 1/1) to give desired Compound (330 mg, yield 65%, purity 95%) as a white solid, which was further separated by SFC (condition, column:

DAICEL CHIRALPAK AD (250 mm*30 mm, 10 um); mobile phase: [CO₂-MeOH (0.1% NH₃H₂O)]; B %: 50%, isocratic elution mode). 3A (140 mg, 235.85 μmol, 28% yield) was obtained as a white oil. 3B (120 mg, 202.16 μmol, 24% yield) was obtained as a white solid.

LCMS: RT=0.525 min, MS cal.: 593.2, [M+H]⁺=594.1

LCMS: RT=0.477 min, MS cal.: 593.2, [M+H]⁺=594.1

SFC: ee %=100.00%

SFC: ee %=99.24%

¹H NMR (400 MHz, CHCl3-d) δ=7.99 (d, J=1.2 Hz, 1H), 7.71 (s, 1H), 7.63 (t, J=7.2 Hz, 1H), 7.53 (t, J=8.0 Hz, 1H), 7.48 (t, J=74.4 Hz, 1H), 7.44 (d, J=7.6 Hz, 1H), 7.36 (dd, J=1.2, 9.2 Hz, 1H), 6.75 (d, J=7.2 Hz, 1H), 6.65 (d, J=8.4 Hz, 1H), 5.54-5.45 (m, 2H), 4.24-4.10 (m, 1H), 4.01 (s, 3H), 3.97 (s, 3H), 3.02-2.85 (m, 1H), 2.84-2.70 (m, 1H), 2.69-2.50 (m, 2H), 2.41-2.25 (m, 1H), 2.00-1.72 (m, 4H), 1.61 (s, 3H)

¹H NMR (400 MHz, CHCl3-d) δ=7.91 (d, J=2.0 Hz, 1H), 7.63 (s, 1H), 7.55 (t, J=7.2 Hz, 1H), 7.45 (t, J=8.0 Hz, 1H), 7.39 (t, J=73.6 Hz, 1H), 7.36 (dd, J=0.8, 8.0 Hz, 1H), 7.29 (dd, J=1.2, 9.6 Hz, 1H), 7.19 (s, 1H), 6.67 (d, J=7.2 Hz, 1H), 6.57 (d, J=8.0 Hz, 1H), 5.42 (s, 2H), 4.18-4.08 (m, 1H), 3.93 (s, 3H), 3.89 (s, 3H), 2.88 (s, 1H), 2.80-2.67 (m, 1H), 2.65-2.44 (m, 2H), 2.23 (s, 1H), 1.92-1.70 (m, 4H), 1.60 (s, 3H)

### General Procedure for Preparation of Scheme 2, Compound 119 (4A):

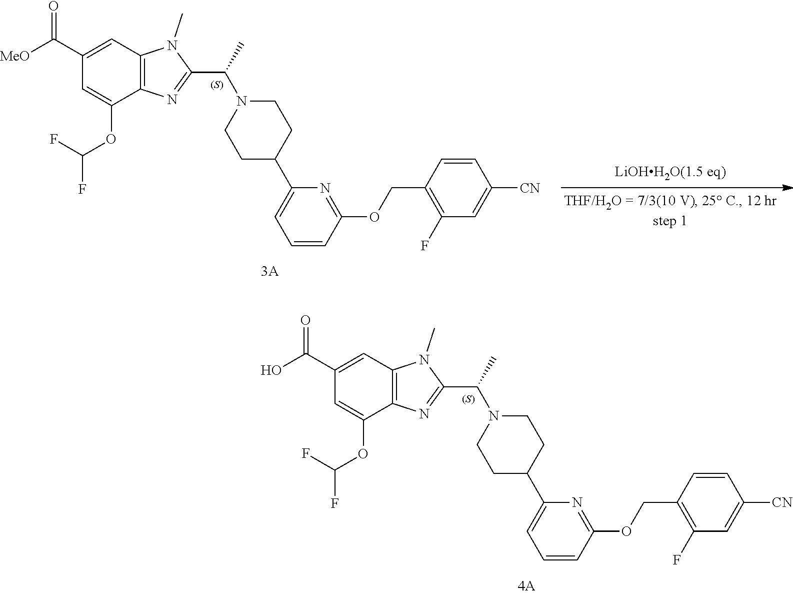

To a solution of 3A (120 mg, 202.16 μmol, 1 eq) in THF (0.7 mL) was added LiOH H₂O (12.72 mg, 303.24 μmol, 1.5 eq) in H₂O (0.3 mL). The mixture was stirred at 25° C. for 12 hr. LC-MS showed 3A was consumed and desired mass was detected. The mixture was purified by prep-HPLC (neutral condition, column: Waters Xbridge Prep OBD C18 150*40 mm*10 um; mobile phase: [H₂O (10 mM NH₄HCO₃)-ACN]; gradient: 30%-60% B over 8.0 min). (S)-2-(1-(4-(6-((4-Cyano-2-fluorobenzyl)oxy)pyridin-2-yl)piperidin-1-yl)ethyl)-4-(difluoromethoxy)-1-methyl-1H-benzo[d]imidazole-6-carboxylic acid (27.44 mg, 46.94 μmol, 23% yield, 99.15% purity) was obtained as a white solid.

LCMS: RT=2.793 min, MS cal.: 579.2, [M+H]⁺=580.2

HPLC: RT=10.764 min, purity: 99.15%

SFC: ee %=100.00%

¹H NMR (400 MHz, MeOH-d4) δ=8.07 (d, J=1.2 Hz, 1H), 7.67-7.62 (m, 2H), 7.61-7.56 (m, 1H), 7.55-7.50 (m, 2H), 7.33 (t, J=74.4 Hz, 1H), 6.82 (d, J=7.2 Hz, 1H), 6.67 (d, J=8.0 Hz, 1H), 5.49 (s, 2H), 4.38 (q, J=6.8 Hz, 1H), 4.03 (s, 3H), 3.06 (t, J=12.8 Hz, 2H), 2.75-2.58 (m, 2H), 2.45 (dt, J=2.4, 11.2 Hz, 1H), 1.93-1.70 (m, 4H), 1.63 (d, J=6.8 Hz, 3H)

### General Procedure for Preparation of Scheme 3, Compound 122 (4B):

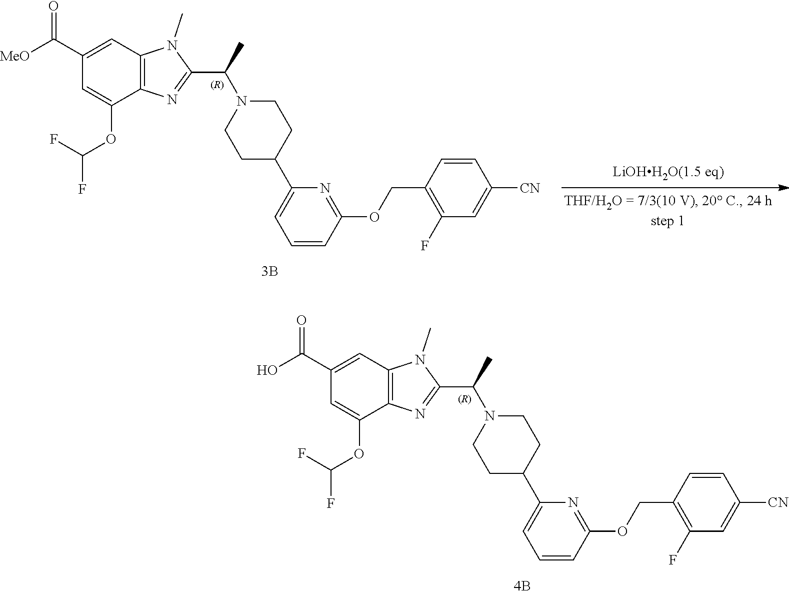

To a solution of 3B (120 mg, 202.16 μmol, 1 eq) in THF (1 mL) was added LiOH H₂O (12.72 mg, 303.24 μmol, 1.5 eq) in H₂O (0.3 mL). The mixture was stirred at 20° C. for 24 hr. LC-MS showed ˜ 30% of 3B remained, and ˜ 66% of desired mass was detected. The mixture was purified by prep-HPLC (neutral condition, column: Waters Xbridge Prep OBD C18 150*40 mm*10 um; mobile phase: [H₂O (10 mM NH₄HCO₃)-ACN]; gradient: 30%-60% B over 8.0 min). (R)-2-(1-(4-(6-((4-Cyano-2-fluorobenzyl)oxy)pyridin-2-yl)piperidin-1-yl)ethyl)-4-(difluoromethoxy)-1-methyl-1H-benzo[d]imidazole-6-carboxylic acid (28.60 mg, 49.35 μmol, 24.41% yield, 98.70% purity) was obtained as a white solid.

LCMS: RT=2.754 min, MS cal.: 579.2, [M+H]⁺=580.2

HPLC: RT=10.833 min, purity: 98.70%

SFC: ee %=99.67%

¹H NMR (400 MHz, MeOH-d4) δ=8.10 (d, J=1.2 Hz, 1H), 7.69-7.63 (m, 2H), 7.61-7.52 (m, 3H), 7.33 (t, J=69.2 Hz, 1H), 6.82 (d, J=7.2 Hz, 1H), 6.67 (d, J=8.4 Hz, 1H), 5.50 (s, 2H), 4.31-4.42 (m, 1H), 4.03 (s, 3H), 2.98-3.12 (m, 2H), 2.72-2.56 (m, 2H), 2.43 (dt, J=11.2 Hz, 1H), 1.96-1.71 (m, 4H), 1.62 (d, J=7.2 Hz, 3H)

### Example A214—Preparation of Compound 118. (S)-2-((4-(2-(5-Chloropyridin-2-yl)-2-methylbenzo[d][1,3]dioxol-4-yl)piperidin-1-yl)methyl)-4-isopropoxy-1-methyl-1H-benzo[d]imidazole-6-carboxylic acid (Compound 118) (6)

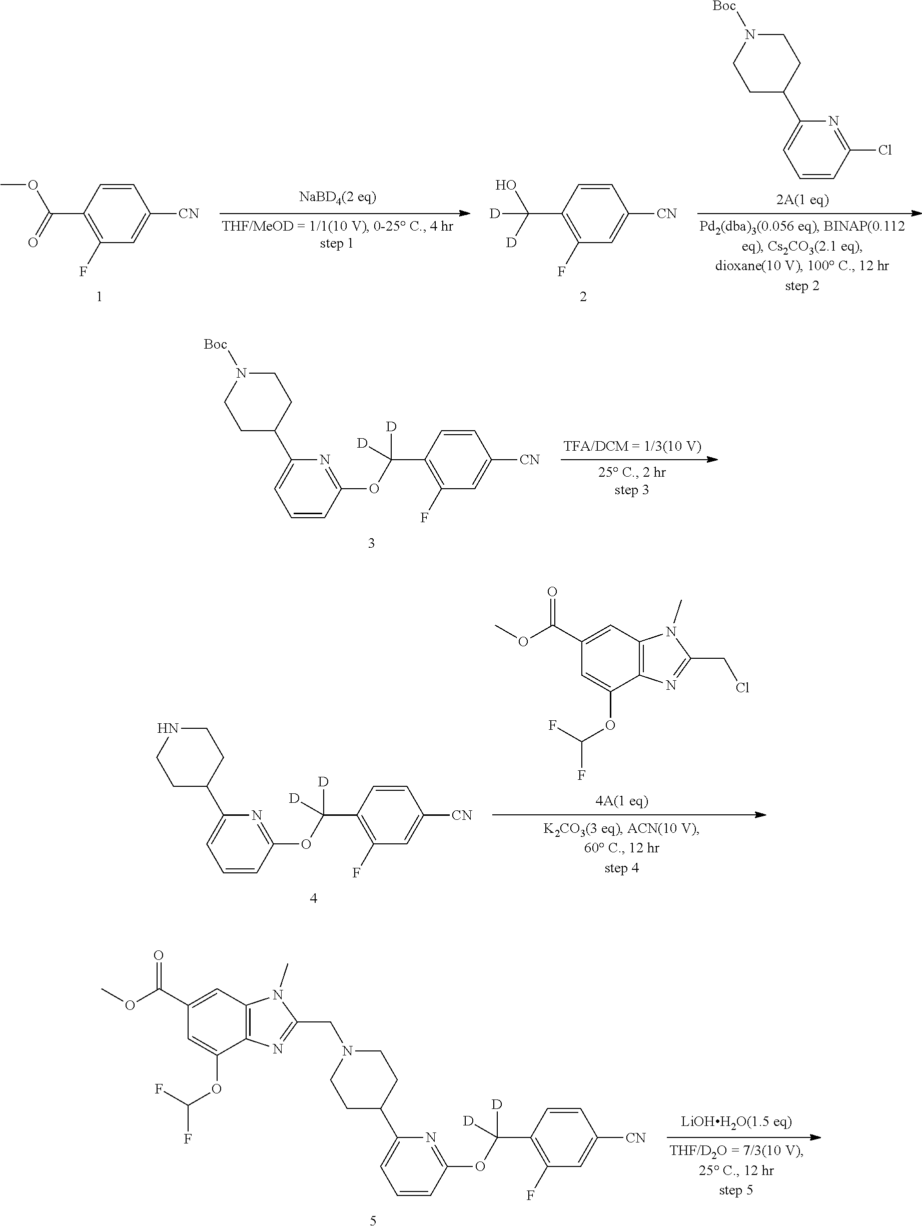

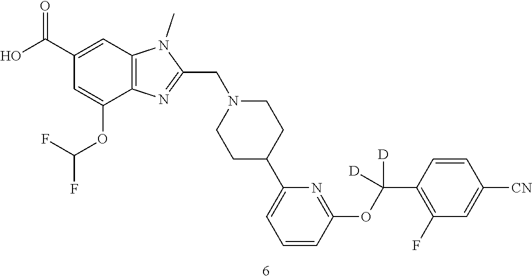 [SMILES: [2H]C([2H])(Oc1cccc(C2CCN(Cc3nc4c(OC(F)F)cc(C(=O)O)cc4n3C)CC2)n1)c1ccc(C#N)cc1F]

### General Procedure for Preparation of Intermediate 2:

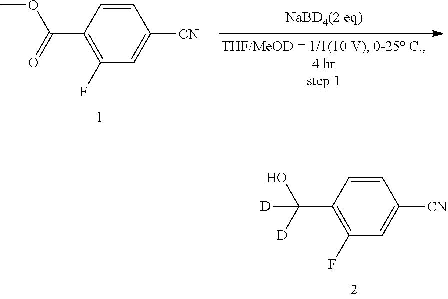

THF (5 mL) and MeOD (5 mL) was charged to the three-necked round bottom flask, then 1 (1 g, 5.58 mmol, 1 eq) was added to the mixture at 25° C. At 0° C. (inner temperature), NaBD₄ (422.36 mg, 11.16 mmol, 2 eq) was added to the reaction mixture at 0° C. within 2 min. After the addition, the mixture was stirred at 25° C. for 12 hr. TLC indicated 1 was consumed completely and one new spot formed. The reaction mixture was added H₂O 20 ml. The mixture was extracted with EtOAc 30 mL (10 mL*3). Then organic phase was combined and washed by brine 20 mL, dried with Na₂SO₄, filtered. The organic phase was concentrated under reduced pressure at 40° C. to give a residue. The residue was purified by column chromatography (SiO₂, Petroleum ether/Ethyl acetate=10/1 to 1/1). 2 (0.69 g, 4.51 mmol, 80.71% yield) was obtained as a white solid.

¹H NMR (400 MHz, CHCl3-d) δ=7.64 (t, J=7.6 Hz, 1H), 7.50 (dd, J=1.2, 7.6 Hz, 1H), 7.35 (dd, J=1.2, 9.2 Hz, 1H)

### General Procedure for Preparation of Intermediate 3:

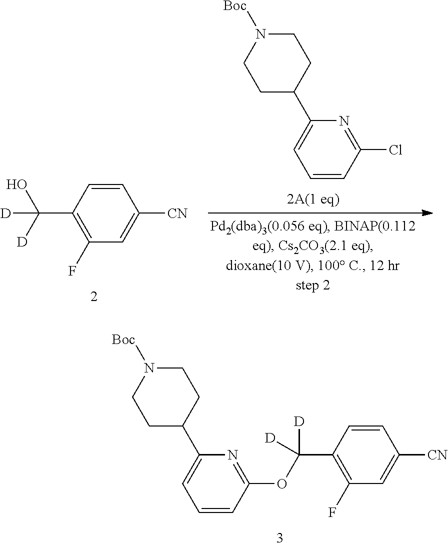 [SMILES: CC(C)(C)OC(=O)N1CCC(c2cccc(Cl)n2)CC1 ; [2H]C([2H])(O)c1ccc(C#N)cc1F ; [2H]C([2H])(Oc1cccc(C2CCN(C(=O)OC(C)(C)C)CC2)n1)c1ccc(C#N)cc1F]

To a solution of 2 (590 mg, 3.85 mmol, 1 eq), 2A (1.14 g, 3.85 mmol, 1 eq) in dioxane (6 mL) was added BINAP (268.67 mg, 431.47 μmol, 0.112 eq), Pd₂(dba)₃ (197.55 mg, 215.74 μmol, 0.056 eq) and Cs₂CO₃ (2.64 g, 8.09 mmol, 2.1 eq). The mixture was stirred at 100° C. for 12 hr. LC-MS showed 2 was consumed completely and desired mass was detected. The residue was diluted with H₂O (20 mL) and extracted with EtOAc 60 mL (20 mL*3). The combined organic layers were washed with brine (20 mL), dried over Na₂SO₄ filtered and concentrated under reduced pressure to give a residue. The residue was purified by column chromatography (SiO₂, Petroleum ether/Ethyl acetate=10/1 to 5/1). 3 (526 mg, 1.27 mmol, 33.02% yield) was obtained as a yellow solid.

LCMS: RT=0.637 min, MS cal.: 413.2, [M+H]⁺=358.1

¹H NMR (400 MHz, DMSO-d₆) δ=7.89-7.79 (m, 1H), 7.73-7.59 (m, 3H), 6.88 (d, J=7.2 Hz, 1H), 6.73 (d, J=8.0 Hz, 1H), 4.10-3.91 (m, 2H), 2.87-2.63 (m, 3H), 1.73 (br d, J=11.2 Hz, 2H), 1.53-1.33 (m, 11H)

### General Procedure for Preparation of Intermediate 4:

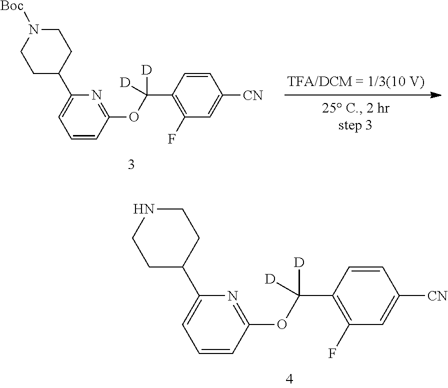 [SMILES: [2H]C([2H])(Oc1cccc(C2CCN(C(=O)OC(C)(C)C)CC2)n1)c1ccc(C#N)cc1F ; [2H]C([2H])(Oc1cccc(C2CCNCC2)n1)c1ccc(C#N)cc1F]

A mixture of 3 (110 mg, 266.03 μmol, 1 eq), TFA (0.3 mL) in DCM (1 mL) was degassed and purged with N₂ for 3 times, and then the mixture was stirred at 25° C. for 2 hr under N₂ atmosphere. TLC indicated Compound 3 was consumed completely and one new spot formed. The reaction was clean according to TLC. The reaction mixture was concentrated under reduced pressure to give a residue. 4 (110 mg, 257.38 μmol, 96.75% yield, TFA) was obtained as a yellow oil.

LCMS: RT=0.375 min, MS cal.: 313.2, [M+H]⁺=314.1

### General Procedure for Preparation of Intermediate 5:

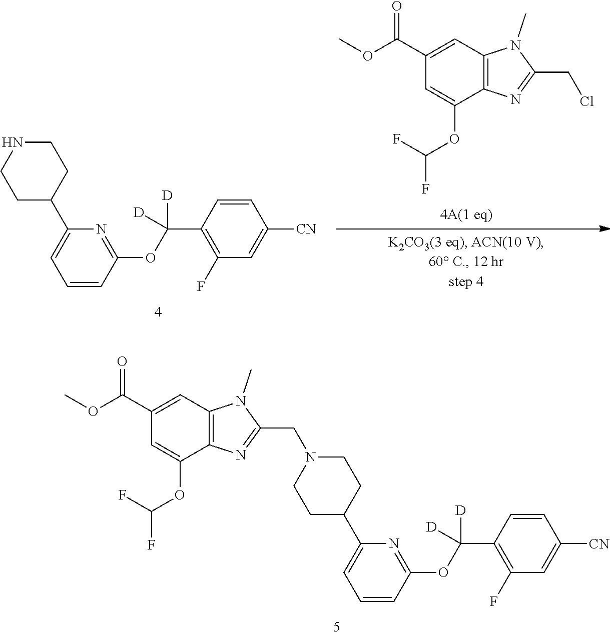 [SMILES: [2H]C([2H])(Oc1cccc(C2CCNCC2)n1)c1ccc(C#N)cc1F ; COC(=O)c1cc(OC(F)F)c2nc(CCl)n(C)c2c1 ; [2H]C([2H])(Oc1cccc(C2CCN(Cc3nc4c(OC(F)F)cc(C(=O)OC)cc4n3C)CC2)n1)c1ccc(C#N)cc1F]

To a solution of 4 (103 mg, 241.00 μmol, 1 eq, TFA), 4A (73.43 mg, 241.00 μmol, 1 eq) in CH₃CN (1 mL) was added K₂CO₃ (99.92 mg, 723.00 μmol, 3 eq). The mixture was stirred at 60° C. for 12 hr. LC-MS showed 4 was consumed completely and desired mass was detected. The residue was diluted with H₂O (10 mL) and extracted with EtOAc 45 mL (15 mL*3). The combined organic layers were washed with brine (10 mL), dried over Na₂SO₄ filtered and concentrated under reduced pressure to give a residue. The residue was purified by column chromatography (SiO₂, Petroleum ether/Ethyl acetate=10/1 to 1/1). 5 (100 mg, 171.95 μmol, 71.35% yield) was obtained as a gray solid.

LCMS: RT=0.459 min, MS cal.: 581.2, [M+H]⁺=582.

¹H NMR (400 MHz, DMSO-d₆) δ=8.12 (d, J=1.2 Hz, 1H), 7.88 (d, J=10.0 Hz, 1H), 7.69 (d, J=3.8 Hz, 2H), 7.62-7.62 (m, 1H), 7.63 (t, J=8.0 Hz, 1H), 7.64 (t, J=74.4 Hz, 1H), 7.53 (s, 1H), 6.87 (d, J=7.3 Hz, 1H), 6.71 (d, J=8.1 Hz, 1H), 3.98 (s, 3H), 3.90 (s, 3H), 3.85 (s, 2H), 2.92 (br d, J=11.2 Hz, 2H), 2.61-2.53 (m, 1H), 2.26-2.15 (m, 2H), 1.78-1.57 (m, 4H)

### General Procedure for Preparation of Compound 118 (6):

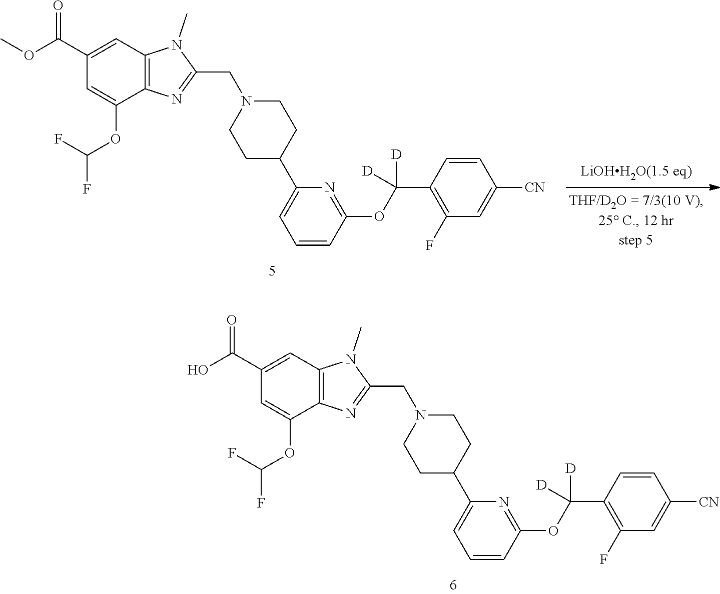 [SMILES: [2H]C([2H])(Oc1cccc(C2CCN(Cc3nc4c(OC(F)F)cc(C(=O)OC)cc4n3C)CC2)n1)c1ccc(C#N)cc1F ; [2H]C([2H])(Oc1cccc(C2CCN(Cc3nc4c(OC(F)F)cc(C(=O)O)cc4n3C)CC2)n1)c1ccc(C#N)cc1F]

To a solution of 5 (100 mg, 171.95 μmol, 1 eq) in THF (0.7 mL) was added LiOH H₂O (10.82 mg, 257.92 μmol, 1.5 eq) in D₂O (0.3 mL). The mixture was stirred at 25° C. for 12 hr. LC-MS showed 5 was consumed completely and desired mass was detected. The reaction mixture was purified directly. The residue was purified by prep-HPLC (neutral condition; column: Waters Xbridge Prep OBD C18 150*40 mm*10 um; mobile phase: [H₂O (10 mM NH₄HCO₃)-ACN]; gradient: 25%-55% B over 8.0 min). (S)-2-((4-(2-(5-Chloropyridin-2-yl)-2-methylbenzo[d][1,3]dioxol-4-yl)piperidin-1-yl)methyl)-4-isopropoxy-1-methyl-1H-benzo[d]imidazole-6-carboxylic acid (29.21 mg, 51.47 μmol, 29.93% yield) was obtained as a white solid.

LCMS: RT=2.667 min, MS cal.: 567.2, [M+H]⁺=568.2

HPLC: RT=10.691 min, purity: 99.2%

¹H NMR (400 MHz, MeOH-d4) δ=8.11 (s, 1H), 7.69 (s, 1H), 7.68-7.64 (m, 1H), 7.62-7.51 (m, 3H), 7.24 (t, J=74.0 Hz, 1H), 6.86-6.82 (m, 1H), 6.67 (d, J=8.3 Hz, 1H), 4.02 (s, 3H), 3.99-3.91 (m, 1H), 3.11-3.02 (m, 2H), 2.71-2.59 (m, 1H), 2.44-2.30 (m, 2H), 1.85 (br d, J=5.0 Hz, 4H)

### Example A215—Preparation of Compound 117. (S)-2-((4-(6-((4-Cyano-2-fluorobenzyl)oxy)pyridin-2-yl)piperidin-1-yl)methyl)-7-fluoro-4-methoxy-1-(oxetan-2-ylmethyl)-1H-benzo[d]imidazole-6-carboxylic acid (Compound 117) (9)

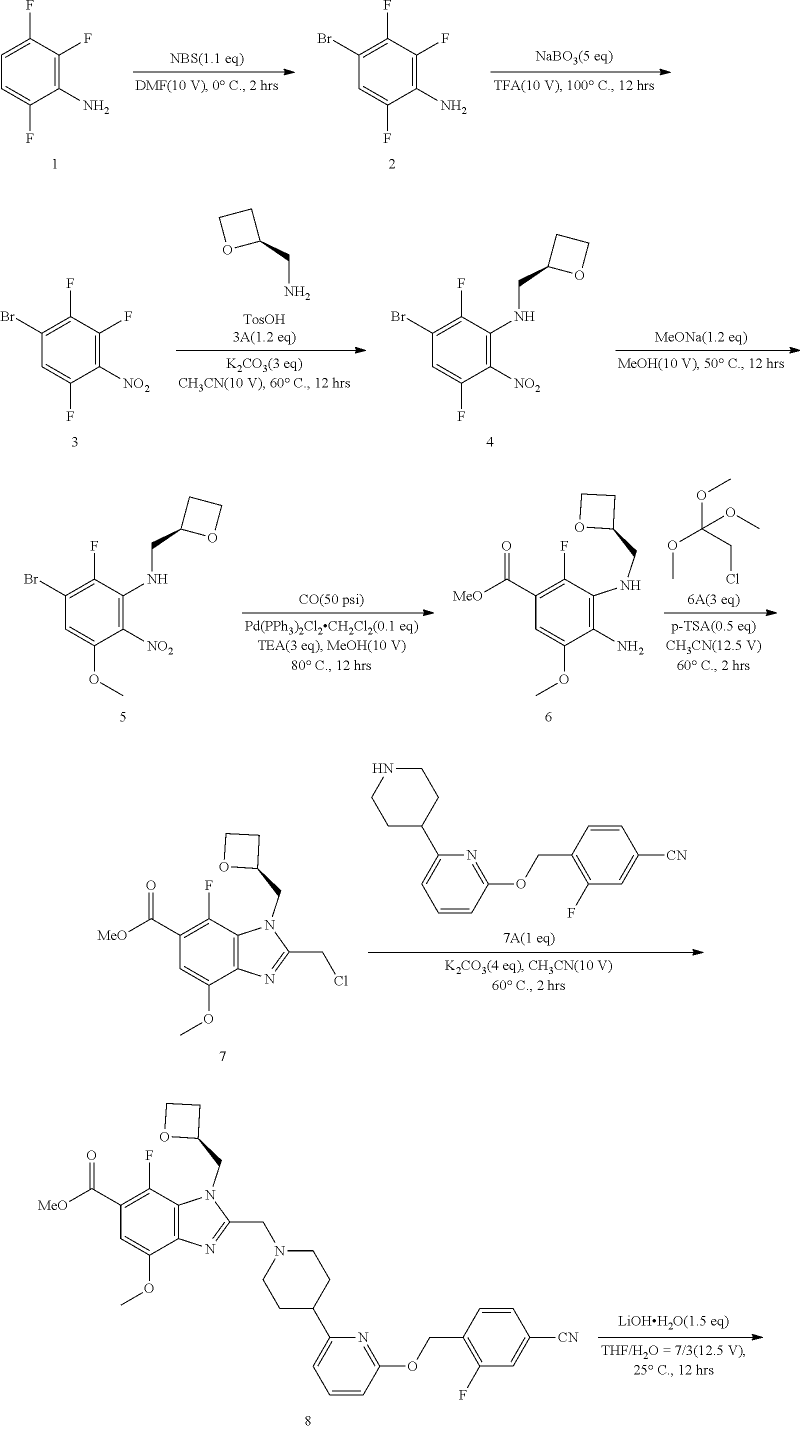

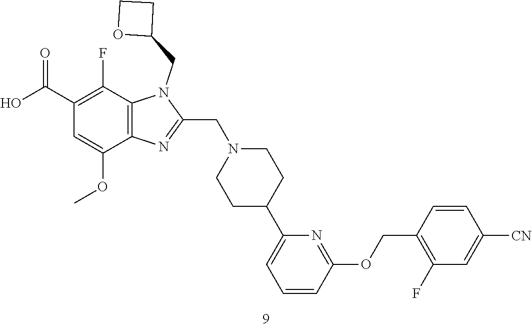 [SMILES: COc1cc(C(=O)O)c(F)c2c1nc(CN1CCC(c3cccc(OCc4ccc(C#N)cc4F)n3)CC1)n2C[C@@H]1CCO1]

### General Procedure for Preparation of Intermediate 2:

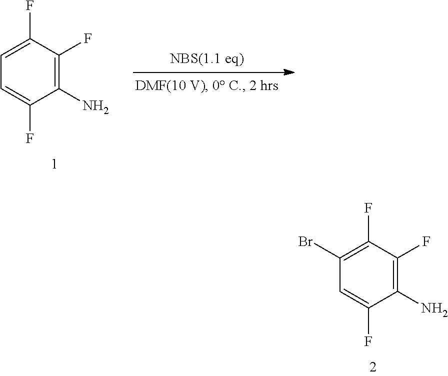 [SMILES: Nc1c(F)ccc(F)c1F ; Nc1c(F)cc(Br)c(F)c1F]

A mixture of 1 (15 g, 101.97 mmol, 1 eq), NBS (19.96 g, 112.17 mmol, 1.1 eq) in DMF (150 mL) was degassed and purged with N₂ for 3 times, and then the mixture was stirred at 0° C. for 2 hrs under N₂ atmosphere. TLC (Petroleum ether/Ethyl acetate=5/1 R_{f}=0.52) showed the starting material was consumed completely. The reaction mixture was poured into water (200 mL) and extracted with ethyl acetate (300 mL*2). The combined organic phase was concentrated in vacuo to give a residue. The residue was purified by column chromatography (SiO₂, Petroleum ether/Ethyl acetate=100/1 to 95/5). 2 (18 g, 79.65 mmol, 78.11% yield) was obtained as a brown oil.

HPLC: RT=2.154 min, purity: 99.14%

¹H NMR (400 MHz, CHCl3-d) δ=7.03 (m, 1H), 3.90 (br s, 2H)

### General Procedure for Preparation of Intermediate 3:

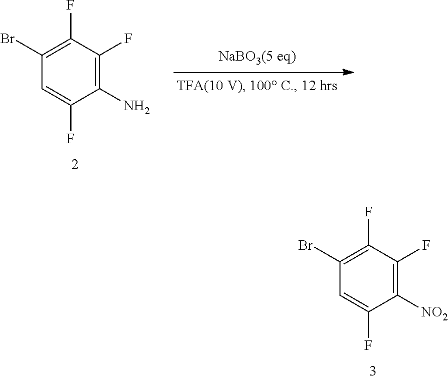 [SMILES: Nc1c(F)cc(Br)c(F)c1F ; O=[N+]([O-])c1c(F)cc(Br)c(F)c1F]

A mixture of 2 (3 g, 13.27 mmol, 1 eq), NaBO₃ (10.21 g, 66.37 mmol, 12.77 mL, 5 eq) in TFA (30 mL) was degassed and purged with N₂ for 3 times, and then the mixture was stirred at 100° C. for 12 hrs under N₂ atmosphere. HPLC (ET43530-1532-P1A) showed the starting material was consumed completely. TLC (Petroleum ether/Ethyl acetate=10/1 R_{f}=0.56) indicated 2 was consumed completely. The reaction mixture was concentrated to give the crude product. The crude product was poured into water (100 mL) and extracted with DCM (100 mL*2). The combined organic phase was concentrated in vacuo to give a residue. The residue was purified by column chromatography (SiO₂, Petroleum ether/Ethyl acetate=1/0 to 49/1). 3 (3 g, 11.72 mmol, 88.29% yield) was obtained as a white solid.

HPLC: RT=2.349 min, purity: 62.28%

¹H NMR (400 MHz, CHCl3-d) δ=7.40 (m, 1H)

### General Procedure for Preparation of Intermediate 4:

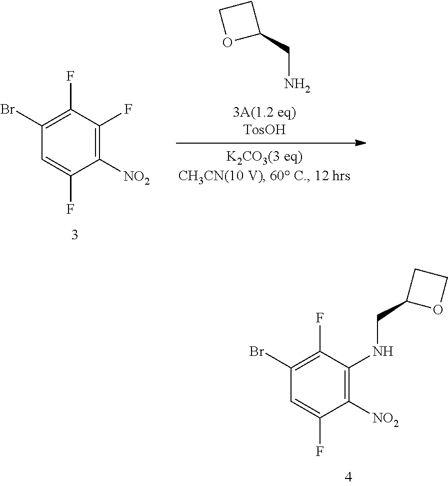 [SMILES: O=[N+]([O-])c1c(F)cc(Br)c(F)c1F ; NC[C@@H]1CCO1 ; O=[N+]([O-])c1c(F)cc(Br)c(F)c1NC[C@H]1CCO1]

A mixture of 3 (2 g, 7.81 mmol, 1 eq), 3A (2.43 g, 9.38 mmol, 1.2 eq, p-TSA), K₂CO₃ (3.24 g, 23.44 mmol, 3 eq) in ACN (20 mL) was degassed and purged with N₂ for 3 times, and then the mixture was stirred at 60° C. for 12 hrs under N₂ atmosphere. TLC (Petroleum ether/Ethyl acetate=5/1 R_{f}=0.41) indicated 3 was consumed completely. The reaction mixture was poured into water (20 mL) and extracted with ethyl acetate (20 mL*2). The combined organic phase was concentrated in vacuo to give a residue. The residue was purified by column chromatography (SiO₂, Petroleum ether/Ethyl acetate=100/1 to 9/1). 4 (1.5 g, 4.64 mmol, 59.42% yield) was obtained as a white solid.

LCMS: RT=0.908 min, MS cal.: 321.8, [M+H]⁺=323.0

¹H NMR (400 MHz, CHCl3-d) δ=6.93-6.82 (m, 1H), 6.75 (dd, J=5.2, 9.9 Hz, 1H), 5.08-4.99 (m, 1H), 4.77-4.69 (m, 1H), 4.57-4.53 (m, 1H), 3.76-3.62 (m, 2H), 2.77-2.65 (m, 1H), 2.60-2.47 (m, 1H)

### General Procedure for Preparation of Intermediate 5:

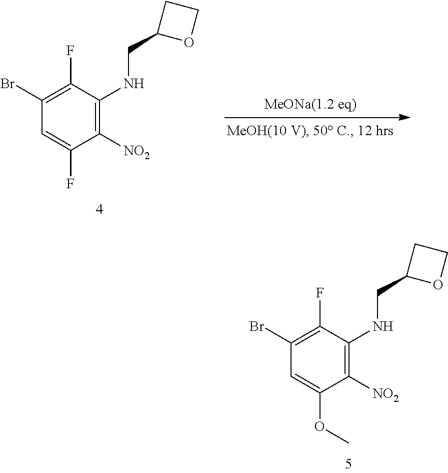 [SMILES: O=[N+]([O-])c1c(F)cc(Br)c(F)c1NC[C@H]1CCO1 ; COc1cc(Br)c(F)c(NC[C@H]2CCO2)c1[N+](=O)[O-]]

A mixture of 4 (0.8 g, 2.48 mmol, 1 eq), MeONa (160.52 mg, 2.97 mmol, 1.2 eq) in MeOH (8 mL) was degassed and purged with N₂ for 3 times, and then the mixture was stirred at 50° C. for 12 hr under N₂ atmosphere. LCMS (ET43530-1543-P1A) showed the starting material was consumed completely. The reaction mixture was poured into water (20 mL) and extracted with ethyl acetate (20 mL*2). The combined organic phase was concentrated in vacuo to give a residue. The crude product was purified by reversed-phase HPLC (column: Waters Xbridge BEH C18 250*50 mm*10 um; mobile phase: [H₂O (10 mM NH₄HCO₃)-ACN]; gradient: 35%-65% B over 10.0 min). 5 (0.53 g, 1.58 mmol, 63.87% yield) was obtained as a yellow solid.

LCMS: RT=1.886 min, MS cal.: 334.0, [M+H]⁺=334.9

¹H NMR (400 MHz, DMSO-d6) δ=6.88-6.83 (m, 1H), 6.05-5.98 (m, 1H), 4.82-4.73 (m, 1H), 4.51-4.41 (m, 1H), 4.34-4.27 (m, 1H), 3.86-3.79 (m, 3H), 3.40-3.34 (m, 2H), 2.62-2.53 (m, 1H), 2.42-2.32 (m, 1H)

### General Procedure for Preparation of Intermediate 6:

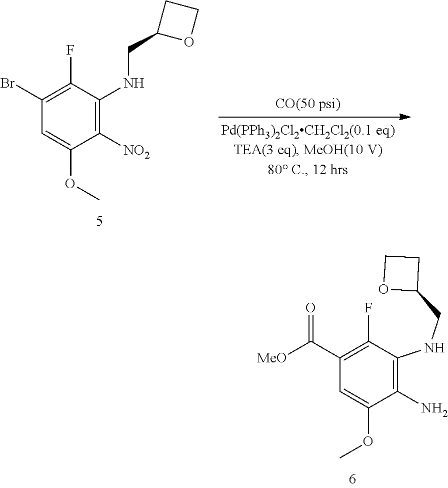 [SMILES: COc1cc(Br)c(F)c(NC[C@H]2CCO2)c1[N+](=O)[O-] ; COC(=O)c1cc(OC)c(N)c(NC[C@@H]2CCO2)c1F]

MeOH (4 mL) and TEA (362.34 mg, 3.58 mmol, 498.40 μL, 3 eq) was charged to the 100 mL 5 (0.4 g, 1.19 mmol, 1 eq) and Pd(dppf)Cl₂·CH₂Cl₂ (97.47 mg, 119.36 μmol, 0.1 eq) was added at 20° C. After the addition, the mixture was degassed and purged with CO for 3 times. The mixture was stirred at 80° C. (50 psi) for 12 hrs. TLC indicated 5 was consumed completely. LCMS (ET43530-1544-P1A1) showed the starting material was consumed completely. The reaction mixture was poured into water (20 mL) and extracted with ethyl acetate (20 mL*2). The combined organic phase was concentrated in vacuo to give a residue. The residue was purified by column chromatography (SiO₂, Petroleum ether/Ethyl acetate=100/1 to 9/1). 6 (0.16 g, 562.82 μmol, 47.15% yield) was obtained as a white solid.

LCMS: RT=1.886 min, MS cal.: 334.0, [M+H]⁺=334.9

¹H NMR (400 MHz, DMSO-d6) δ=6.91 (d, J=6.4 Hz, 1H), 5.53 (s, 2H), 4.83-4.70 (m, 1H), 4.57-4.49 (m, 1H), 4.47-4.39 (m, 1H), 4.29-4.18 (m, 1H), 3.78 (s, 3H), 3.76 (s, 3H), 3.23-3.04 (m, 2H), 2.62-2.54 (m, 1H), 2.48-2.38 (m, 2H).

### General Procedure for Preparation of Intermediate 7:

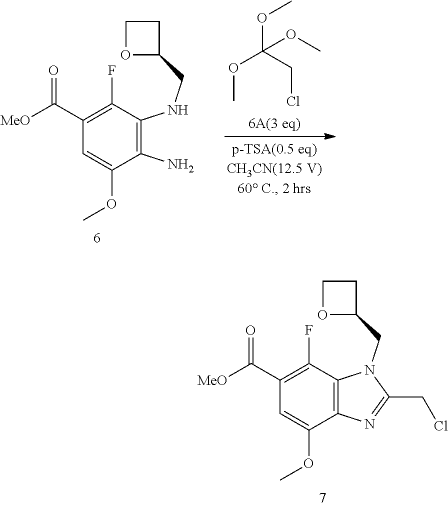

A mixture of 6 (0.08 g, 281.41 μmol, 1 eq), 6A (130.51 mg, 844.23 μmol, 113.78 μL, 3 eq), p-TSA (24.23 mg, 140.70 μmol, 0.5 eq) in ACN (1 mL) was degassed and purged with N₂ for 3 times, and then the mixture was stirred at 60° C. for 2 hrs under N₂ atmosphere. LCMS (ET43530-1552-P1A) showed the starting material was consumed completely. The reaction mixture was concentrated to give the crude product. 7 (0.096 g, crude) was obtained as a white solid.

LCMS: RT=1.020 min, MS cal.: 342.0, [M+H]⁺=342.9

### General Procedure for Preparation of Intermediate 8:

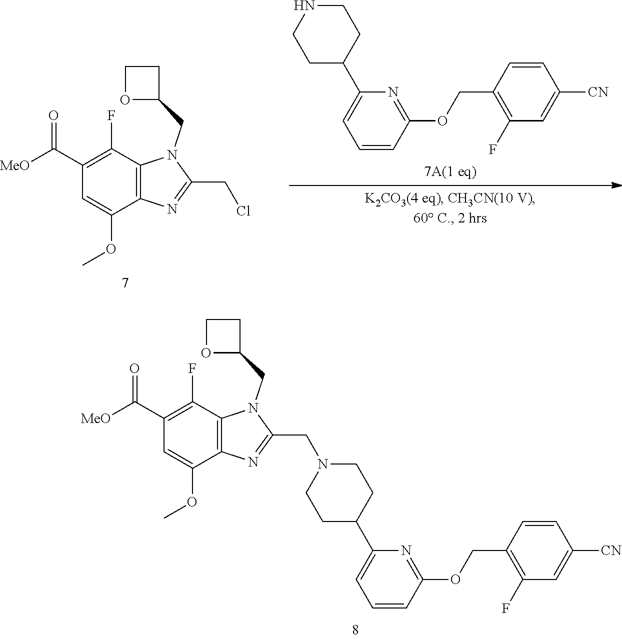 [SMILES: COC(=O)c1cc(OC)c2nc(CCl)n(C[C@@H]3CCO3)c2c1F ; N#Cc1ccc(COc2cccc(C3CCNCC3)n2)c(F)c1 ; COC(=O)c1cc(OC)c2nc(CN3CCC(c4cccc(OCc5ccc(C#N)cc5F)n4)CC3)n(C[C@@H]3CCO3)c2c1F]

A mixture of 7 (0.096 g, 280.09 μmol, 1 eq), 7A (synthesized from Int 6) (87.21 mg, 280.09 μmol, 1 eq), K₂CO₃ (154.84 mg, 1.12 mmol, 4 eq) in ACN (1 mL) was degassed and purged with N₂ for 3 times, and then the mixture was stirred at 60° C. for 2 hrs under N₂ atmosphere. TLC (Petroleum ether/Ethyl acetate=0/1 R_{f}=0.26) indicated 7 was consumed completely. The reaction mixture was poured into water (20 mL) and extracted with ethyl acetate (20 mL*2). The combined organic phase was concentrated in vacuo to give a residue. The residue was purified by column chromatography (SiO₂, Petroleum ether/Ethyl acetate=100/1 to 0/1). 8 (80 mg, 129.53 μmol, 46.24% yield) was obtained as a white solid.

LCMS: RT=1.479 min, MS cal.: 617.2, [M+H]⁺=618.1

### General Procedure for Preparation of Compound 117 (9):

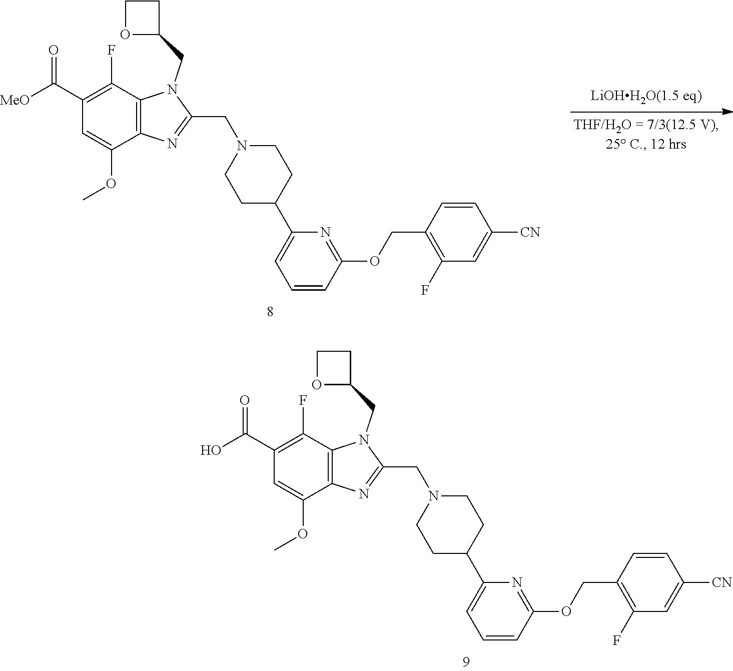 [SMILES: COC(=O)c1cc(OC)c2nc(CN3CCC(c4cccc(OCc5ccc(C#N)cc5F)n4)CC3)n(C[C@@H]3CCO3)c2c1F ; COc1cc(C(=O)O)c(F)c2c1nc(CN1CCC(c3cccc(OCc4ccc(C#N)cc4F)n3)CC1)n2C[C@@H]1CCO1]

A mixture of 8 (80 mg, 129.53 μmol, 1 eq) LiOH·H₂O (8.15 mg, 194.29 μmol, 1.5 eq) in THF (0.7 mL) H₂O (0.3 mL) was degassed and purged with N₂ for 3 times, and then the mixture was stirred at 25° C. for 12 hrs under N₂ atmosphere. LCMS (ET43530-1561-P1A) showed the starting material was consumed completely. The reaction mixture was purified directly. The crude product was purified by reversed-phase HPLC (column: Waters Xbridge Prep OBD C18 150*40 mm*10 um; mobile phase: [H₂O (10 mM NH₄HCO₃)-ACN]; gradient: 20%-60% B over 8.0 min). (S)-2-((4-(6-((4-Cyano-2-fluorobenzyl)oxy)pyridin-2-yl)piperidin-1-yl)methyl)-7-fluoro-4-methoxy-1-(oxetan-2-ylmethyl)-1H-benzo[d]imidazole-6-carboxylic acid (28.26 mg, 43.95 μmol, 33.93% yield, 93.87% purity) was obtained as a white solid.

LCMS: RT=2.583 min, MS cal.: 603.2, [M+H]⁺=604.3

HPLC: RT=10.112 purity: 93.87%

¹H NMR (400 MHz, MeOH-d4) δ=7.67 (t, J=7.6 Hz, 1H), 7.63-7.51 (m, 3H), 7.11 (d, J=4.8 Hz, 1H), 6.85 (d, J=7.2 Hz, 1H), 6.68 (d, J=8 Hz, 1H), 5.52 (s, 2H), 5.32-5.22 (m, 1H), 5.00 (dd, J=6.8, 15.4 Hz, 1H), 4.82 (br s, 1H), 4.69-4.60 (m, 1H), 4.44 (td, J=6.0, 9.1 Hz, 1H), 4.12-4.05 (m, 1H), 4.03-3.97 (m, 4H), 3.12 (br d, J=12 Hz, 1H), 3.05 (br d, J=12 Hz, 1H), 2.87-2.75 (m, 1H), 2.67 (br t, J=7.2 Hz, 1H), 2.59-2.48 (m, 1H), 2.48-2.34 (m, 2H), 1.94-1.80 (m, 4H)

### Example A216—Preparation of Compound 114. 2-((4-(6-((4-Cyano-2-fluorobenzyl)oxy)pyridin-2-yl)piperidin-1-yl)methyl)-4-hydroxy-1-methyl-1H-benzo[d]imidazole-6-carboxylic acid (Compound 114) (2)

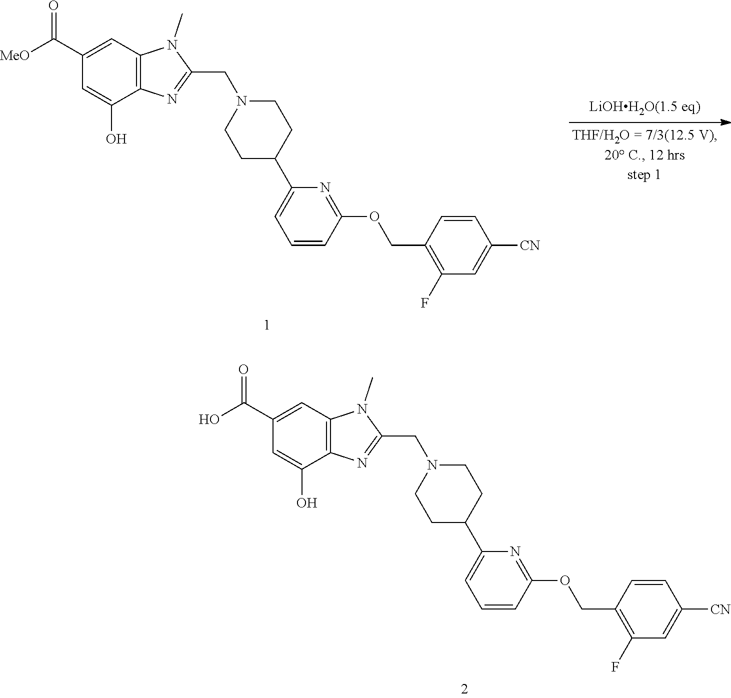 [SMILES: COC(=O)c1cc(O)c2nc(CN3CCC(c4cccc(OCc5ccc(C#N)cc5F)n4)CC3)n(C)c2c1 ; Cn1c(CN2CCC(c3cccc(OCc4ccc(C#N)cc4F)n3)CC2)nc2c(O)cc(C(=O)O)cc21]

### General Procedure for Preparation of Compound 114 (2):

A mixture of 1 (50 mg, 94.42 μmol, 1 eq) (synthesized from Int 7), LiOH·H₂O (8.32 mg, 198.28 μmol, 2.1 eq) in THF (0.7 mL) and H₂O (0.3 mL) was degassed and purged with N₂ for 3 times, and then the mixture was stirred at 25° C. for 12 hr under N₂ atmosphere. LCMS showed ˜17% of 1 remained. Several new peaks were shown on LC-MS and ˜71% of desired Compound was detected. The reaction mixture was filtered and concentrated under reduced pressure to give a residue in DMSO. The residue was purified by prep-HPLC (column: Waters Xbridge Prep OBD C18 150*40 mm*10 um; mobile phase: [H₂O (10 mM NH₄HCO₃)-ACN]; gradient: 15%-65% B over 8.0 min). 2-((4-(6-((4-Cyano-2-fluorobenzyl)oxy)pyridin-2-yl)piperidin-1-yl)methyl)-4-hydroxy-1-methyl-1H-benzo[d]imidazole-6-carboxylic acid (21.97 mg, 42.62 μmol, 45.14% yield) was obtained as a white solid.

LCMS: RT=2.517 min, MS cal.: 547.2, [M+H]⁺=548.2

HPLC: RT=9.027 min, purity: 98.14%

¹H NMR (400 MHz, DMSO-d₆) δ=7.89 (d, J=10.2 Hz, 1H), 7.70 (d, J=3.6 Hz, 2H), 7.65 (t, J=7.6 Hz, 1H), 7.59 (s, 1H), 7.18 (s, 1H), 6.88 (d, J=7.2 Hz, 1H), 6.72 (d, J=8.0 Hz, 1H), 5.46 (s, 2H), 3.88 (s, 3H), 3.80 (s, 2H), 2.93 (br d, J=11.6 Hz, 2H), 2.63-2.57 (m, 1H), 2.18 (br t, J=10.4 Hz, 2H), 1.80-1.60 (m, 4H)

### Example A217—Preparation of Compound 111. 2-((4-(3-((4-Cyano-2-fluorobenzyl)oxy)phenyl)piperidin-1-yl)methyl)-4-(difluoromethoxy)-1-methyl-1H-benzo[d]imidazole-6-carboxylic acid (Compound 111) (7)

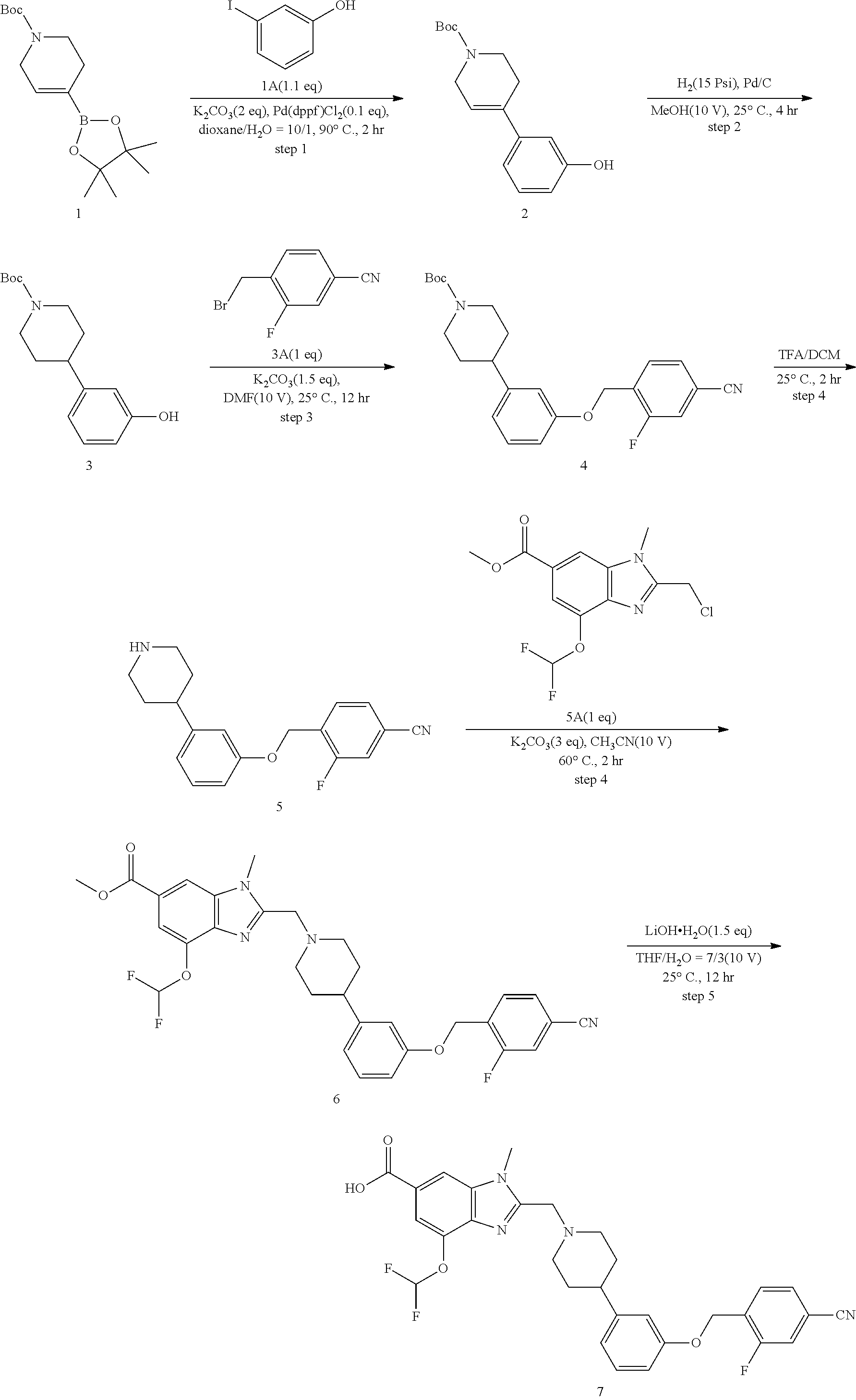

### General Procedure for Preparation of Intermediate 2:

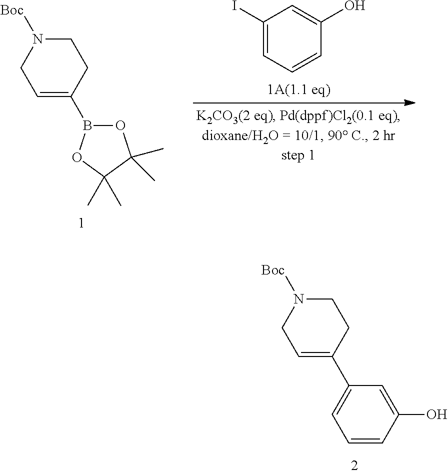 [SMILES: CC(C)(C)OC(=O)N1CC=C(B2OC(C)(C)C(C)(C)O2)CC1 ; Oc1cccc(I)c1 ; CC(C)(C)OC(=O)N1CC=C(c2cccc(O)c2)CC1]

2 reactions were carried out in parallels. To a mixture of 1 (1.28 g, 4.13 mmol, 1 eq) and 1A (1 g, 4.55 mmol, 1.1 eq) in dioxane (30 mL) and H₂O (3 mL) was added K₂CO₃ (1.14 g, 8.26 mmol, 2 eq) and Pd(dppf)Cl₂ (302.35 mg, 413.21 μmol, 0.1 eq) under nitrogen. The mixture was stirred at 90 C for 2 hours under nitrogen. TLC indicated 1 was consumed completely and one new spot formed. (Petroleum ether/Ethyl acetate=3/1). 2 reactions were combined for workup. The reaction mixture was poured into water (50 mL). The aqueous phase was extracted with ethyl acetate (20 mL*3). The combined organic phase was washed with brine (20 mL*2), dried with anhydrous Na₂SO₄, filtered and concentrated in vacuum. 2 (1.4 g, 5.08 mmol, 61.53% yield) was a colorless oil.

¹H NMR (400 MHz, DMSO-d6) δ=9.36 (s, 1H), 7.12 (t, J=8.0 Hz, 1H), 6.85 (d, J=8.0 Hz, 1H), 6.79 (s, 1H), 6.66 (d, J=7.6 Hz, 1H), 6.06 (br s, 1H), 4.01-3.91 (m, 2H), 3.51 (br t, J=5.6 Hz, 2H), 2.46-2.32 (m, 2H), 1.48-1.38 (m, 9H)

### General Procedure for Preparation of Intermediate 3:

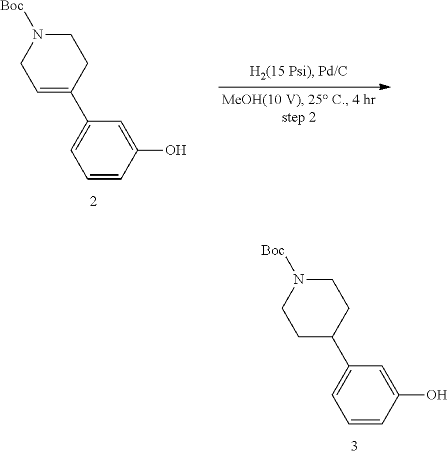 [SMILES: CC(C)(C)OC(=O)N1CC=C(c2cccc(O)c2)CC1 ; CC(C)(C)OC(=O)N1CCC(c2cccc(O)c2)CC1]

MeOH (6 mL) was charged to the Hydrogenating flask, then Pd/C (139.14 mg) and 2 (600 mg, 2.18 mmol, 1 eq) were added to the reaction mixture. After the addition, the suspension was degassed under vacuum and purged with hydrogen several times. The mixture was stirred at 25° C. for 12 hr under hydrogen (15 psi). LCMS indicated Reactant 1 was consumed completely. The mixture was filtered by celite, the filter cake was washed with MeOH (12 mL). The combined organic phase was dried with anhydrous Na₂SO₄, filtered and concentrated in vacuum. 3 (580 mg, 2.09 mmol, 95.96% yield) was a white solid.

LCMS: RT=1.676 min, MS cal.: 277.2, [M−55]⁺=222.1

¹H NMR (400 MHz, DMSO-d6) δ=9.24 (s, 1H), 7.07 (t, J=7.6 Hz, 1H), 6.67-6.55 (m, 3H), 4.12-3.96 (m, 2H), 2.78 (br s, 2H), 2.62-2.52 (m, 1H), 1.71 (br d, J=12.8 Hz, 2H), 1.47-1.36 (m, 12H)

### General Procedure for Preparation of Intermediate 4:

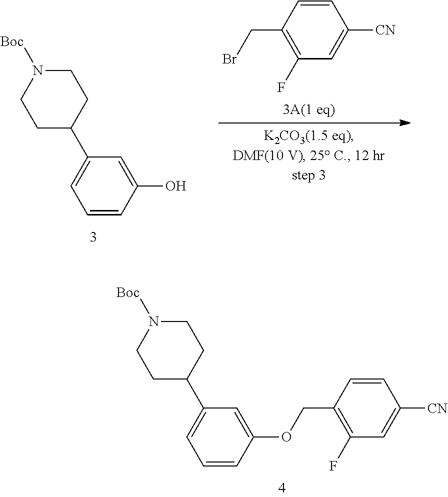 [SMILES: CC(C)(C)OC(=O)N1CCC(c2cccc(O)c2)CC1 ; N#Cc1ccc(CBr)c(F)c1 ; CC(C)(C)OC(=O)N1CCC(c2cccc(OCc3ccc(C#N)cc3F)c2)CC1]

To a solution of 3 (600 mg, 2.16 mmol, 1 eq), 3A (463.01 mg, 2.16 mmol, 1 eq) in DMF (6 mL) was added K₂CO₃ (448.46 mg, 3.24 mmol, 1.5 eq). The mixture was stirred at 25° C. for 12 hr. LCMS 3 was consumed completely. The residue was poured into water (10 mL). The aqueous phase was extracted with ethyl acetate (10 mL*3). The combined organic phase was washed with brine (10 mL*2), dried with anhydrous Na₂SO₄, filtered, and concentrated in vacuum. The residue was purified by column chromatography (SiO₂, Petroleum ether/Ethyl acetate=1/0 to 4/1). 4 (788 mg, 1.92 mmol, 88.74% yield) was a white solid.

LCMS: RT=2.185 min, MS cal.: 410.2, [M−55]⁺=355.1

¹H NMR (400 MHz, DMSO-d6) δ=7.91 (d, J=9.2 Hz, 1H), 7.79-7.69 (m, 2H), 7.22 (t, J=8.0 Hz, 1H), 6.95-6.82 (m, 3H), 5.22 (s, 2H), 4.15-3.97 (m, 2H), 2.78 (br s, 2H), 2.73-2.56 (m, 1H), 1.73 (br d, J=12.8 Hz, 2H), 1.57-1.45 (m, 2H), 1.45-1.33 (m, 9H)

### General Procedure for Preparation of Intermediate 5:

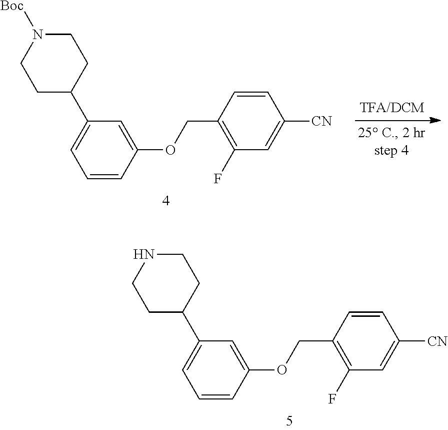 [SMILES: CC(C)(C)OC(=O)N1CCC(c2cccc(OCc3ccc(C#N)cc3F)c2)CC1 ; N#Cc1ccc(COc2cccc(C3CCNCC3)c2)c(F)c1]

To a solution of 4 (200 mg, 487.23 μmol, 1 eq) in DCM (2 mL) was added TFA (555.56 mg, 4.87 mmol, 361.93 μL, 10 eq). The mixture was stirred at 25° C. for 2 hr. LCMS indicated 4 was consumed completely. The reaction mixture was concentrated under reduced pressure to remove TFA and DCM. 5 (206 mg, 485.40 μmol, 99.62% yield, TFA) was a white solid.

LCMS: RT=1.207 min, MS cal.: 310.2, [M+H]⁺=311.1

¹H NMR (400 MHz, DMSO-d6) δ=8.62 (br s, 1H), 8.38 (br s, 1H), 7.93 (d, J=10.0 Hz, 1H), 7.76 (d, J=3.6 Hz, 2H), 7.28 (t, J=8.0 Hz, 1H), 6.96-6.81 (m, 3H), 5.23 (s, 2H), 3.43-3.37 (m, 1H), 3.05-2.92 (m, 2H), 2.89-2.77 (m, 1H), 1.97-1.89 (m, 2H), 1.77 (dq, J=4.0, 13.1 Hz, 2H)

### General Procedure for Preparation of Intermediate 6:

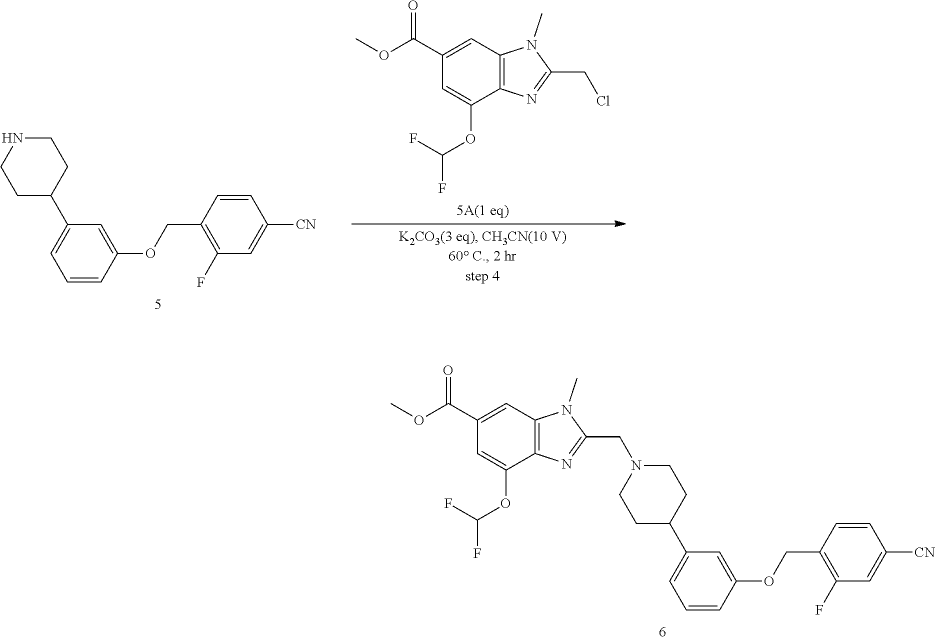 [SMILES: N#Cc1ccc(COc2cccc(C3CCNCC3)c2)c(F)c1 ; COC(=O)c1cc(OC(F)F)c2nc(CCl)n(C)c2c1 ; COC(=O)c1cc(OC(F)F)c2nc(CN3CCC(c4cccc(OCc5ccc(C#N)cc5F)c4)CC3)n(C)c2c1]

To a solution of 5A (110 mg, 361.04 μmol, 1 eq) and 5 (183.86 mg, 433.25 μmol, 1.2 eq, TFA) in CH₃CN (1 mL) was added K₂CO₃ (149.69 mg, 1.08 mmol, 3 eq). The mixture was stirred at 60° C. for 2 hr. LCMS indicated 5 was consumed completely. The residue was poured into water (10 mL). The aqueous phase was extracted with ethyl acetate (10 mL*3). The combined organic phase was washed with brine (10 mL*2), dried with anhydrous Na₂SO₄, filtered, and concentrated in vacuum. The residue was purified by column chromatography (SiO₂, Petroleum ether/Ethyl acetate=1/0 to 3/1). 6 (180 mg, 311.11 μmol, 86.17% yield) was a white solid.

LCMS: RT=1.609 min, MS cal.: 578.4, [M+H]⁺=579.2

¹H NMR (400 MHz, DMSO-d6) δ=8.15-8.05 (m, 1H), 7.90 (br d, J=9.6 Hz, 1H), 7.79-7.70 (m, 2H), 7.65 (t, J=74.0 Hz, 1H), 7.53 (s, 1H), 7.20 (br t, J=8.0 Hz, 1H), 6.91 (br s, 1H), 6.85 (br d, J=7.6 Hz, 2H), 5.20 (s, 2H), 3.97 (s, 3H), 3.90 (s, 3H), 3.85 (br s, 2H), 3.33-3.26 (m, 1H), 2.94 (br d, J=10.6 Hz, 2H), 2.25-2.13 (m, 2H), 1.79-1.59 (m, 4H)

### General Procedure for Preparation of Compound 111 (7):

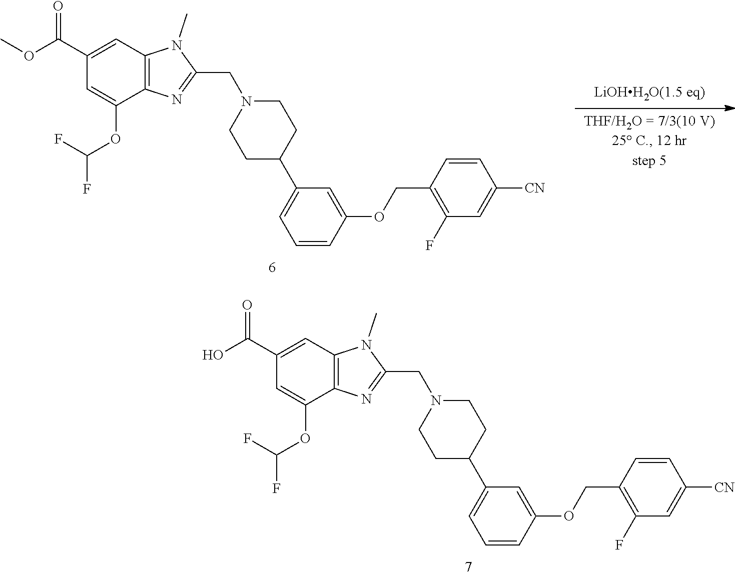 [SMILES: COC(=O)c1cc(OC(F)F)c2nc(CN3CCC(c4cccc(OCc5ccc(C#N)cc5F)c4)CC3)n(C)c2c1 ; Cn1c(CN2CCC(c3cccc(OCc4ccc(C#N)cc4F)c3)CC2)nc2c(OC(F)F)cc(C(=O)O)cc21]

A mixture of 6 (80 mg, 138.27 μmol, 1 eq), LiOH·H₂O (8.70 mg, 207.40 μmol, 1.5 eq) in THF (0.7 mL) and H₂O (0.3 mL) was degassed and purged with N₂ for 3 times, and then the mixture was stirred at 25° C. for 12 hr under N₂ atmosphere. LCMS (ET57960-776-P1A3, RT=0.685 min) showed 79% of product was detected. The reaction mixture was concentrated to give a residue. The residue was purified by prep-HPLC (neutral condition column: Waters Xbridge Prep OBD C18 150*40 mm*10 um; mobile phase: [H₂O (10 mM NH₄HCO₃)-ACN]; gradient: 15%-45B over 8.0 min). 2-((4-(3-((4-Cyano-2-fluorobenzyl)oxy)phenyl)piperidin-1-yl)methyl)-4-(difluoromethoxy)-1-methyl-1H-benzo[d]imidazole-6-carboxylic acid (29.25 mg, 48.55 μmol, 35.11% yield, 93.70% purity) was obtained as a white solid.

LCMS: RT=2.696 min, MS cal.: 564.6, [M+H]⁺=565.2

HPLC: RT=11.240 min, purity: 93.70%

¹H NMR (400 MHz, MeOH-d4) δ=8.10 (d, J=1.2 Hz, 1H), 7.73 (t, J=7.6 Hz, 1H), 7.68 (s, 1H), 7.62-7.56 (m, 2H), 7.24 (t, J=74.4 Hz, 1H), 7.21 (t, J=8.0 Hz, 1H), 6.91-6.80 (m, 3H), 5.20 (s, 2H), 4.02 (s, 3H), 3.96 (s, 2H), 3.08 (d, J=11.6 Hz, 2H), 2.63-2.52 (m, 1H), 2.38 (dt, J=2.8, 11.4 Hz, 2H), 1.88-1.72 (m, 4H)

### Example A218—Preparation of Compound 110. (S)-2-((4-(6-((4-Cyano-2-fluorobenzyl)oxy)pyridin-2-yl)piperidin-1-yl)methyl)-4-(difluoromethoxy)-1-((1,1-dioxidothietan-2-yl)methyl)-1H-benzo[d]imidazole-6-carboxylic acid (Compound 110) (13)

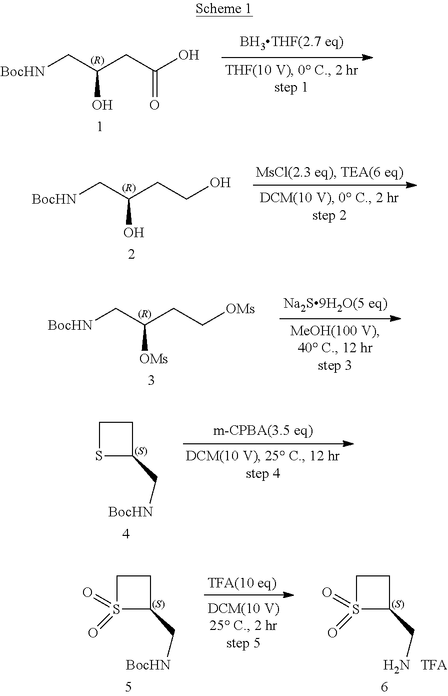

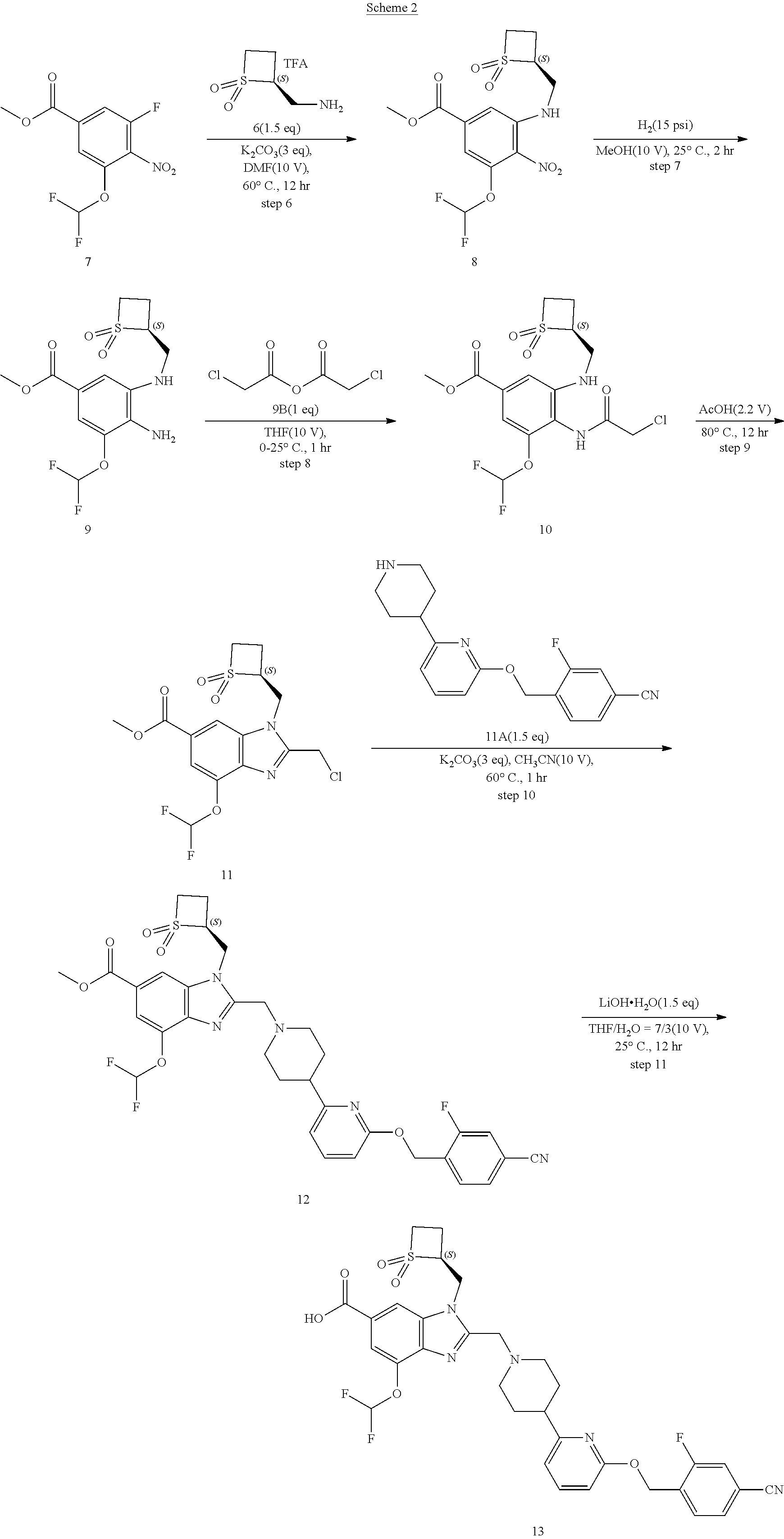

### General Procedure for Preparation of Scheme 1, Intermediate 2:

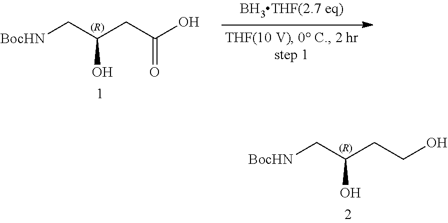

The reaction was set up in 4 batches.

THF (16 mL) was charged to a 250 mL three-necked round bottom flask, then 1 (1.62 g, 7.39 mmol, 1 eq) was added at 0° C. At 0° C. (inner temperature), BH₃·THF (1 M, 19.95 mL, 2.7 eq) was dropwise added to the reaction mixture at 0° C. within 10 min. After the addition, the mixture was stirred at 0° C. for 2 hr. TLC (Petroleum ether/Ethyl acetate=0/1, KMnO₄, R_{f}=0.43) indicated 1 was consumed completely. The reaction mixture was quenched with MeOH (20 ml), the mixture was stirred at 80° C. for 12 hr. Then HCl (1 M, 3 mL) was added and the reaction mixture was extracted with EtOAc (10 mL*5). The combined organic layers were washed with saturated NaHCO₃ (10 mL), dried over Na₂SO₄, filtered, and concentrated under reduced pressure to give a residue. Four reactions were combined for purification. The crude product was combined to ET62365-467 for further purification. The residue was purified by column chromatography (SiO₂, Petroleum ether/Ethyl acetate=10/1 to Ethyl acetate/Methanol=0/1). 2 (4.4 g, 21.44 mmol, 72% yield) was obtained as a white oil.

¹H NMR (400 MHz, DMSO-d₆) δ=6.62 (br s, 1H), 4.50 (d, J=5.2 Hz, 1H), 4.32 (t, J=4.8 Hz, 1H), 3.58-3.42 (m, 3H), 2.98-2.81 (m, 2H), 1.52 (dtd, J=3.6, 7.2, 13.6 Hz, 1H), 1.37 (s, 9H)

### General Procedure for Preparation of Scheme 1, Intermediate 3:

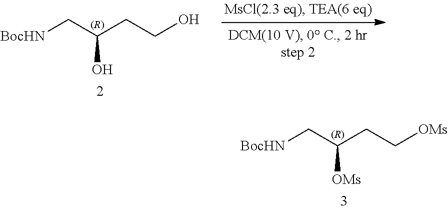

DCM (24 mL) was charged to a 250 mL three-necked round bottom flask, then 2 (2.4 g, 11.69 mmol, 1 eq) and TEA (7.10 g, 70.16 mmol, 9.77 mL, 6 eq) was added at 0° C. At 0° C. (inner temperature), MsCl (3.08 g, 26.89 mmol, 2.08 mL, 2.3 eq) was added dropwise to the reaction mixture at 0° C. After the addition, the mixture was stirred at 0° C. for 2 hr. LCMS (ET62365-475-P1A, product RT=1.249 min) showed 2 was consumed completely. The reaction mixture was quenched by addition saturated NaHCO₃ (20 mL) at 15° C., and then extracted with DCM (20 mL*3). The combined organic layers were washed with NH₄Cl (20 mL*2), dried over Na₂SO₄, filtered and concentrated under reduced pressure to give a residue. 3 (4.2 g, crude) was obtained as a yellow oil.

LCMS: RT=1.259 min. MS cal.: 361.1

### General Procedure for Preparation of Scheme 1, Intermediate 4:

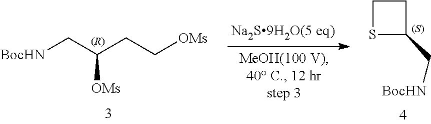

To a solution of 3 (4.2 g, 11.62 mmol, 1 eq) in MeOH (420 mL) was added Na₂S·9H₂O (13.96 g, 58.10 mmol, 9.78 mL, 5 eq). The mixture was stirred at 40° C. for 12 hr. TLC (Petroleum ether/Ethyl acetate=3/1, KMnO₄, R_{f}=0.43) indicated 3 was consumed completely. The reaction mixture was concentrated under reduced pressure to remove MeOH. The residue was added H₂O (30 mL) and extracted with EtOAc (30 Ml*3). The combined organic layers added Na₂SO₄, filtered and concentrated under reduced pressure to give a residue. The residue was purified by column chromatography (SiO₂, Petroleum ether/Ethyl acetate=9/1 to 0/1). 4 (1.7 g, 8.36 mmol, 72% yield) was obtained as a yellow oil.

¹H NMR (400 MHz, DMSO-d₆) δ=7.03-6.93 (m, 1H), 3.65-3.53 (m, 1H), 3.28-3.20 (m, 1H), 3.19-3.11 (m, 1H), 3.09-3.02 (m, 1H), 2.95 (dt, J=5.2, 9.2 Hz, 1H), 2.82 (dtd, J=5.2, 8.4, 12.0 Hz, 1H), 2.58-2.51 (m, 1H), 1.36 (s, 9H)

### General Procedure for Preparation of Intermediate 5:

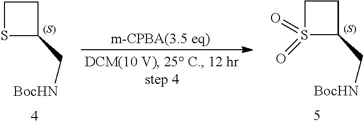

DCM (20 mL) was charged to a three-necked round bottom flask, then 4 (1.5 g, 7.38 mmol, 1 eq) was added to the mixture at 25° C. At 25° C. (inner temperature), m-CPBA (5.24 g, 25.82 mmol, 85% purity, 3.5 eq) was added in portions to the reaction mixture at 25° C. within 0.1 hr. After the addition, the mixture was stirred at 25° C. for 12 hr. TLC (Petroleum ether/Ethyl acetate=2/1, I₂, R_{f}=0.24) indicated 4 was consumed completely. The mixture was washed with saturated NaHCO₃ (20 ml*3) and extracted with DCM (20 mL). Then organic layer was washed with Na₂SO₃ (20 ml*2). The combined organic layers were dried with Na₂SO₄, filtered. The organic phase was concentrated under reduced pressure at 30° C. to give a residue. The residue was purified by column chromatography (SiO₂, Petroleum ether/Ethyl acetate=10/1 to Ethyl acetate/Methanol=0/1). 5 (1.3 g, 5.52 mmol, 75% yield) was obtained as a white solid.

¹H NMR (400 MHz, DMSO-d₆) δ=7.21-6.99 (m, 1H), 4.54-4.41 (m, 1H), 4.10-3.99 (m, 1H), 3.98-3.87 (m, 1H), 3.47-3.37 (m, 1H), 3.29-3.20 (m, 1H), 2.28-2.14 (m, 1H), 1.70 (tdd, J=8.4, 10.4, 11.6 Hz, 1H), 1.39-1.37 (m, 9H)

### General Procedure for Preparation of Scheme 1, Intermediate 6:

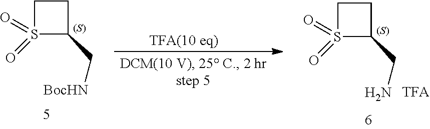

A mixture of 5 (500 mg, 2.12 mmol, 1 eq), TFA (2.42 g, 21.25 mmol, 1.58 mL, 10 eq) in DCM (5 mL) was degassed and purged with N₂ for 3 times, and then the mixture was stirred at 25° C. for 2 hr under N₂ atmosphere. TLC (Petroleum ether/Ethyl acetate=0/1, I₂, R_{f}=0.24) indicated 5 was consumed completely. The reaction mixture was concentrated under reduced pressure to remove DCM. 6 (529 mg, crude, TFA) was obtained as a yellow oil.

### General Procedure for Preparation of Scheme 2, Intermediate 8:

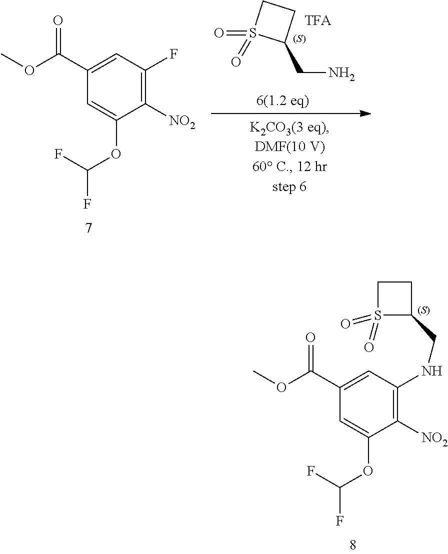

A mixture of 7 (450 mg, 1.70 mmol, 1 eq), 6 (507.55 mg, 2.04 mmol, 1.2 eq, TFA), K₂CO₃ (703.69 mg, 5.09 mmol, 3 eq) in DMF (5 mL) was degassed and purged with N₂ for 3 times, and then the mixture was stirred at 60° C. for 12 hr under N₂ atmosphere. LCMS (ET62365-499-P1A, product: RT=1.114 min) showed 7 was consumed completely. TLC (Petroleum ether/Ethyl acetate=1/1, R_{f}=0.24) indicated 7 was consumed completely. The mixture was diluted with brine (20 ml) and extracted with EtOAc (20 mL*4). Then combined organic layers were dried over Na₂SO₄, filtered, and concentrated under reduced pressure to give a residue. The residue was purified by column chromatography (SiO₂, Petroleum ether/Ethyl acetate=50/1 to Ethyl acetate/Methanol=0/1). 8 (480 mg, 1.26 mmol, 74% yield) was obtained as an orange oil.

LCMS: RT=1.114 min. MS cal.: 380.0; 381.1, [M+H]⁺=380.9

¹H NMR (400 MHz, DMSO-d₆) δ=7.56-7.56 (m, 1H), 7.41 (t, J=72.4 Hz, 1H), 7.37 (s, 1H), 7.10 (s, 1H), 6.77 (t, J=5.2 Hz, 1H), 4.69 (quin, J=8.0 Hz, 1H), 4.16-3.96 (m, 2H), 3.89 (s, 3H), 3.83 (td, J=7.6, 14.8 Hz, 1H), 3.55 (td, J=5.2, 14.0 Hz, 1H), 2.28-2.14 (m, 2H), 1.93-1.78 (m, 2H)

### General Procedure for Preparation of Scheme 2, Intermediate 9:

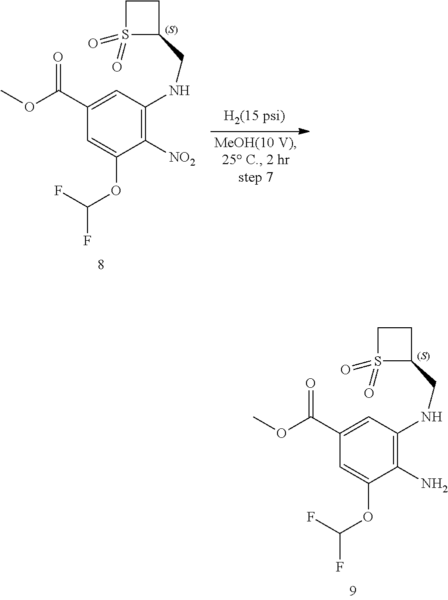

MeOH (5 mL) and was charged to a round bottom flask, then 8 (420 mg, 1.10 mmol, 1 eq) was added to the mixture at 20° C. Pd/C (105 mg) was added to the reaction mixture at 20° C. After the addition, the mixture was stirred at 25° C. for 2 hr. LCMS (ET64759-443-PIP, product: RT=0.392 min) showed the 8 was consumed completely. The suspension was filtered through a pad of Celite, and filter cake was washed with MeOH 10 mL*3. The combined filtrate was concentrated to give a product. 8 (380 mg, 1.08 mmol, 98.22% yield) was obtained as a yellow oil.

### General Procedure for Preparation of Scheme 2, Intermediate 10:

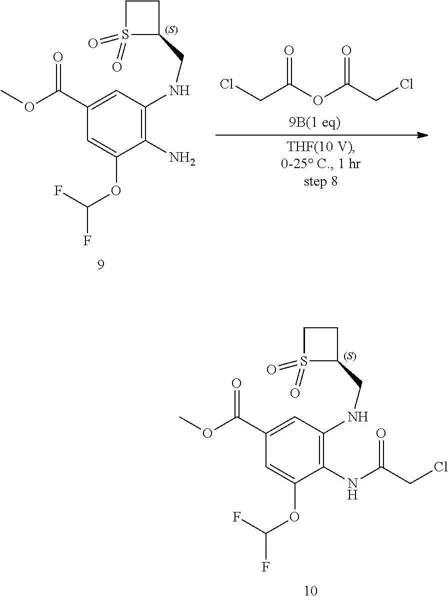

To a solution of 9 (380 mg, 1.08 mmol, 1 eq) in THF (4 mL) was added 9B (185.45 mg, 1.08 mmol, 1 eq) at 0° C. The mixture was stirred at 25° C. for 2 hr. LCMS (product: RT=0.384 min) showed the 9 was consumed completely. The reaction mixture was quenched by addition H₂O 5 mL at 20° C. and extracted with EtOAc 5 mL*3. The combined organic layers were washed with brine 5 mL*2, dried over Na₂SO₄, filtered, and concentrated under reduced pressure to give a residue. 10 (450 mg, crude) was obtained as a yellow oil.

### General Procedure for Preparation of Scheme 2, Intermediate 11:

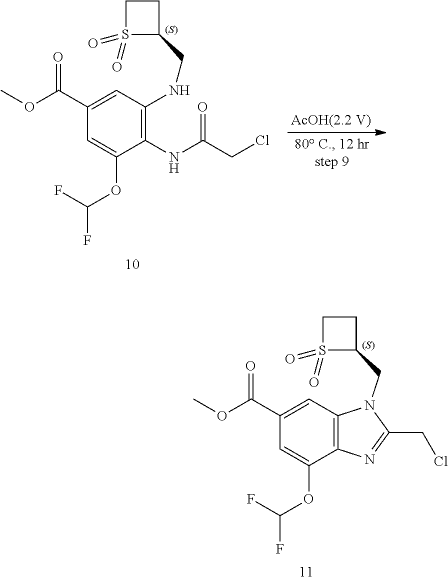

A mixture of 10 (450 mg, 1.05 mmol, 1 eq) in AcOH (1 mL) was degassed and purged with N₂ for 3 times, and then the mixture was stirred at 80° C. for 12 hr under N₂ atmosphere. LCMS (product: RT=0.430 min) showed the 10 was consumed completely. The reaction mixture was quenched by addition H₂O 10 mL at 20° C. and extracted with EtOAc 10 mL*3. The combined organic layers were washed with brine 10 mL*2, dried over Na₂SO₄, filtered and concentrated under reduced pressure to give a residue. The residue was purified by column chromatography (SiO₂, Petroleum ether/Ethyl acetate=5/1 to 1/1). 11 (280 mg, 684.92 μmol, 64.96% yield) was obtained as a white solid.

LCMS: RT=0.428 min, MS cal.: 408.0, [M+H]⁺=409.0

¹H NMR (400 MHz, DMSO-d6) δ=8.35 (d, J=0.8 Hz, 1H), 7.62 (t, J=73.6 Hz, 1H), 7.60 (s, 1H), 5.29-5.13 (m, 2H), 5.05-4.93 (m, 2H), 4.92-4.82 (m, 1H), 4.17-4.08 (m, 2H), 3.91 (s, 3H), 2.45-2.30 (m, 1H), 1.98-1.90 (m, 1H)

### General Procedure for Preparation of Scheme 2, Intermediate 12:

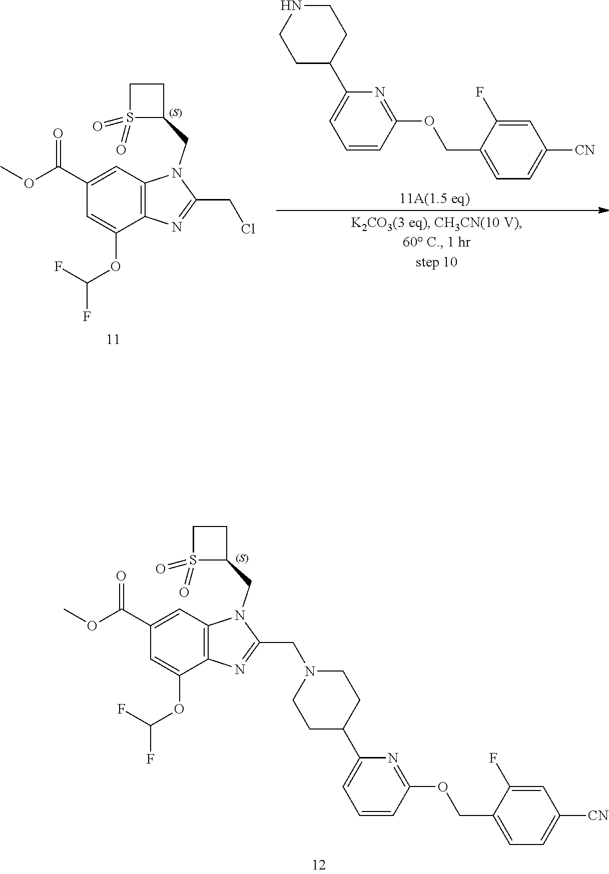

To a solution of 11 (100 mg, 244.62 μmol, 1 eq) 11A (114.24 mg, 366.92 μmol, 1.5 eq) in CH₃CN (1 mL) was added K₂CO₃ (101.42 mg, 733.85 μmol, 3 eq). The mixture was stirred at 60° C. for 1 hr. LCMS (product: RT=0.452 min) showed the 11 was consumed completely. The reaction mixture was concentrated under reduced pressure to remove solvent. The residue was added H₂O 5 mL and extracted with EtOAc 5 mL*3. The combined organic layers were washed with brine 5 mL*2, dried over Na₂SO₄, filtered and concentrated under reduced pressure to give a residue. The residue was purified by column chromatography (SiO₂, Petroleum ether/Ethyl acetate=1/1 to 0/1). 12 (110 mg, 160.89 μmol, 65.77% yield) was obtained as a white solid.

LCMS: RT=0.462 min, MS cal.: 683.2, [M+H]⁺=684.2

### General Procedure for Preparation of Compound 110 (13):

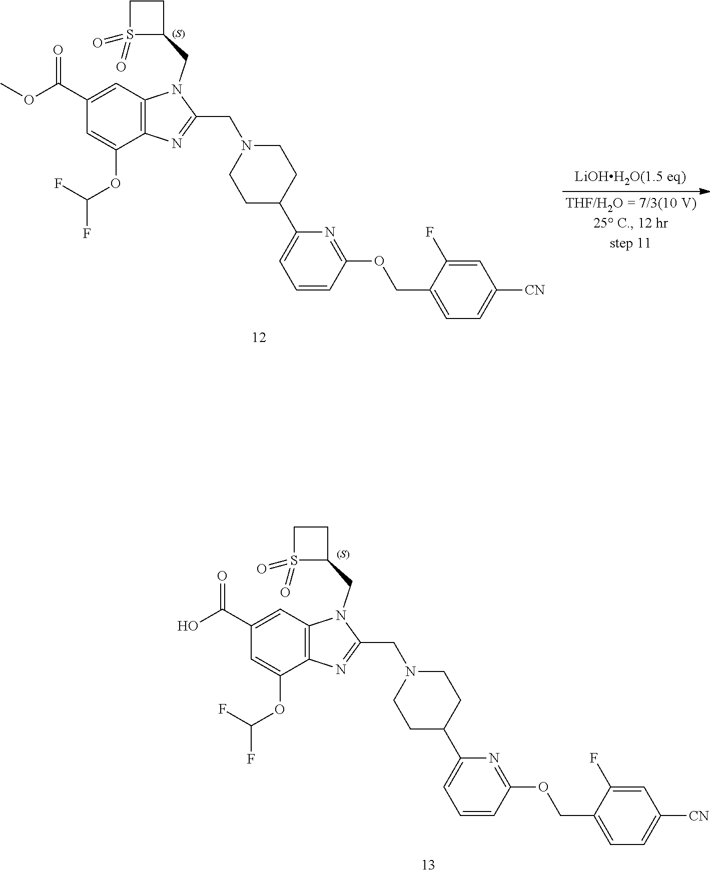

To a solution of 12 in THF (0.7 mL) was added LiOH·H₂O (9.21 mg, 219.40 μmol, 1.5 eq) in H₂O (0.3 mL). The mixture was stirred at 25° C. for 12 hr under N₂ atmosphere. LCMS (product: RT=1.129 min) showed the 12 was consumed completely. The reaction mixture was quenched by addition aq. citric acid (0.5 M) to adjust pH=7-8 at 20° C. The mixture was purified by prep-HPLC (column: Waters Xbridge Prep OBD C18 150*40 mm*10 um; mobile phase: [H₂O (10 mM NH₄HCO₃)—CH₃CN]; gradient: 10%-40% B over 8.0 min). (S)-2-((4-(6-((4-Cyano-2-fluorobenzyl)oxy)pyridin-2-yl)piperidin-1-yl)methyl)-4-(difluoromethoxy)-1-((1,1-dioxidothietan-2-yl)methyl)-1H-benzo[d]imidazole-6-carboxylic acid (28.26 mg, 41.35 μmol, 28.27% yield, 97.98% purity) was obtained as a white solid.

LCMS: RT=2.714 min, MS cal.: 669.2, [M+H]⁺=670.2

HPLC: RT=11.020 min.

¹H NMR (400 MHz, MeOH-d4) δ=8.23 (d, J=0.8 Hz, 1H), 7.73-7.65 (m, 2H), 7.62-7.52 (m, 3H), 7.24 (t, J=74.4 Hz, 1H), 6.85 (d, J=7.2 Hz, 1H), 6.68 (d, J=8.4 Hz, 1H), 5.52 (s, 2H), 5.24-5.12 (m, 2H), 4.83-4.79 (m, 1H), 4.19-4.08 (m, 2H), 4.07-3.98 (m, 1H), 3.89 (d, J=14.0 Hz, 1H), 3.14 (d, J=11.6 Hz, 1H), 2.98 (d, J=11.6 Hz, 1H), 2.72-2.60 (m, 1H), 2.51-2.27 (m, 3H), 2.16-2.03 (m, 1H), 1.95-1.79 (m, 4H)

### Example A219—Preparation of Compound 109. (S)-4-(tert-Butoxy)-2-((4-(6-((4-cyano-2-fluorobenzyl)oxy)pyridin-2-yl)piperidin-1-yl)methyl)-1-(oxetan-2-ylmethyl)-1H-benzo[d]imidazole-6-carboxylic acid (Compound 109) (8)

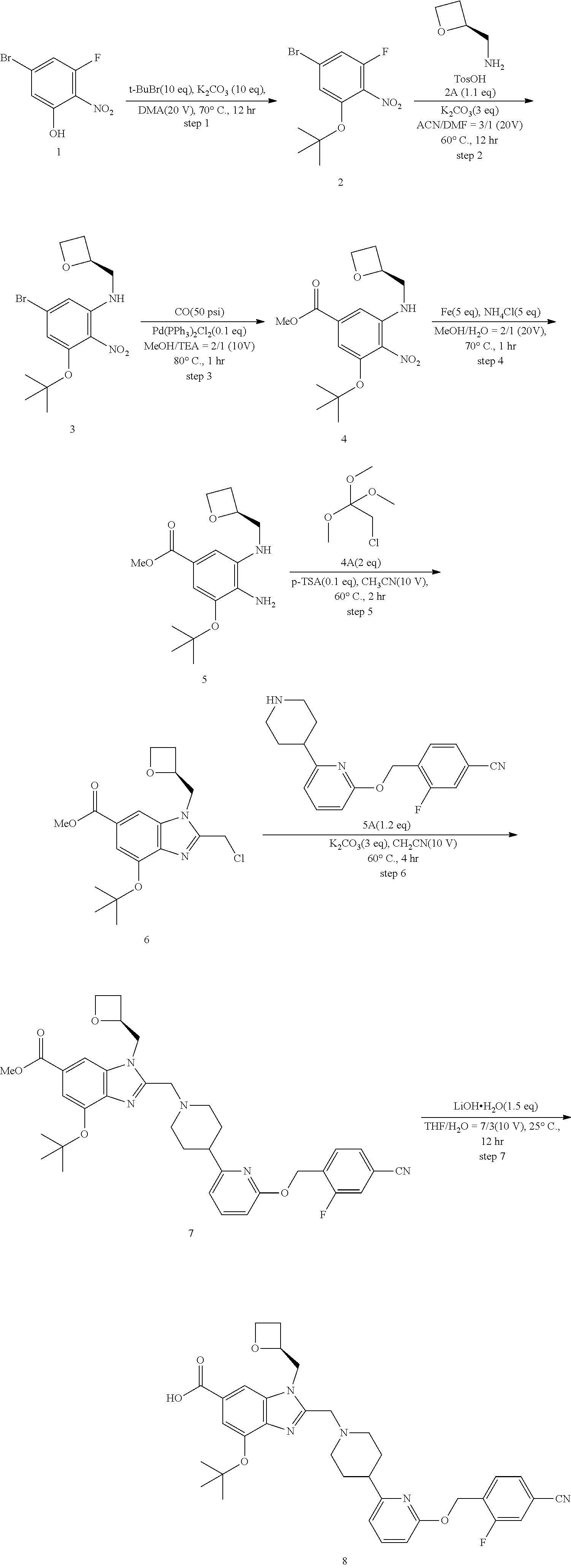

### General Procedure for Preparation of Intermediate 2:

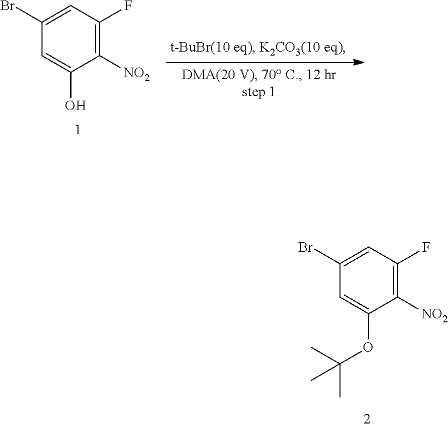 [SMILES: O=[N+]([O-])c1c(O)cc(Br)cc1F ; CC(C)(C)Oc1cc(Br)cc(F)c1[N+](=O)[O-]]

DMA (60 mL) was charged to a 100 mL sealed tube, then 1 (3 g, 12.71 mmol, 1 eq), t-BuBr (17.42 g, 127.12 mmol, 14.76 mL, 10 eq) and K₂CO₃ (17.57 g, 127.12 mmol, 10 eq) was added at 25° C. under N₂. After the addition, the mixture was stirred at 70° C. in sealed tube (15 psi) for 12 hr. TLC indicated ˜30% of 1 was remained and one major new spot with lower polarity was detected. The reaction was cooled to 25° C., then diluted with PE 50 mL and filtered. The filtrate was added H₂O 100 mL, extracted with PE 300 mL (100 mL*3). The combined organic layers were washed by H₂O 250 mL (50 mL*5), brine 100 mL (50 mL*2), filtered and concentrated to give a product without further purification. 2 (2.6 g, 8.90 mmol, 70.02% yield) was obtained as a yellow oil.

¹H NMR (400 MHz, DMSO-d6) δ=7.68 (dd, J=2.0, 9.2 Hz, 1H), 7.49 (t, J=1.6 Hz, 1H), 1.39 (s, 9H)

### General Procedure for Preparation of Intermediate 3:

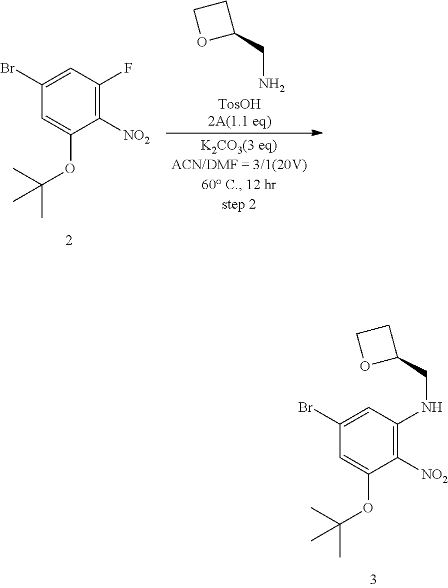

A mixture of 2 (280 mg, 958.57 μmol, 1 eq), 2A (273.44 mg, 1.05 mmol, 1.1 eq, p-TSA), K₂CO₃ (397.44 mg, 2.88 mmol, 3 eq) in ACN (3 mL) and DMF (1 mL) was degassed and purged with N₂ for 3 times, and then the mixture was stirred at 60° C. for 12 hrs under N₂ atmosphere. TLC indicated 2 was consumed completely and one new spot formed. The reaction was clean according to TLC. The residue mixture was diluted with H₂O (20 mL) and extracted with EtOAc (20 mL*3). The combined organic layers were washed with brine (20 mL), dried over Na₂SO₄, filtered and concentrated under reduced pressure to give a residue. The residue was purified by column chromatography (SiO₂, Petroleum ether/Ethyl acetate=100/1 to 9/1, TLC-Ethyl acetate/Methanol=5/1, Product R_{f}=0.41). 3 (200 mg, 551.20 μmol, 57.50% yield, 99% purity) was obtained as a yellow solid.

LCMS: RT=1.356 min, MS cal.: 358.0, 356.0, [M+H]⁺=358.9, 360.9

¹H NMR (400 MHz, CHLOROFORM-d) δ=6.67 (d, J=1.6 Hz, 1H), 6.63 (d, J=1.6 Hz, 1H), 5.11-5.02 (m, 1H), 4.76-4.68 (m, 1H), 4.58 (td, J=6.0, 9.2 Hz, 1H), 3.39 (d, J=4.4 Hz, 2H), 2.79-2.67 (m, 1H), 2.55 (tdd, J=7.2, 9.1, 11.2 Hz, 1H), 1.42 (s, 9H)

### General Procedure for Preparation of Intermediate 4:

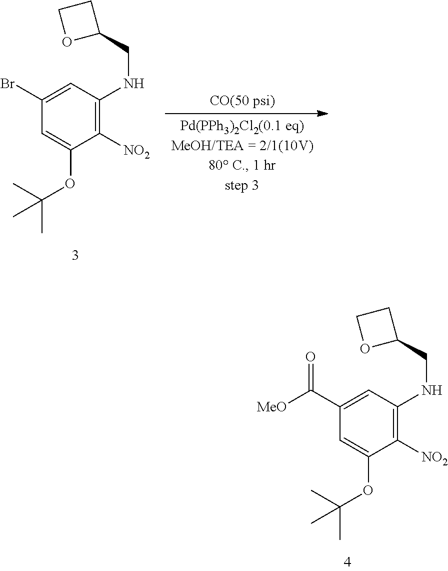 [SMILES: CC(C)(C)Oc1cc(Br)cc(NC[C@@H]2CCO2)c1[N+](=O)[O-] ; "CO(50 psi)" ; COC(=O)c1cc(NC[C@@H]2CCO2)c([N+](=O)[O-])c(OC(C)(C)C)c1]

Equip a 35 mL hydrogenated bottle. MeOH (1.4 mL) and TEA (0.7 mL) was charged to the 35 mL Hydrogenated bottle, then 3 (200 mg, 556.77 μmol, 1 eq) and Pd(PPh₃)₂Cl₂ (39.08 mg, 55.68 μmol, 0.1 eq) was added at 20° C. After the addition, the mixture was degassed and purged with CO for 3 times. The mixture was stirred at 80° C. (50 psi) for 1 hr. TLC indicated 3 was consumed completely and one new spot formed. The reaction was clean according to TLC. The residue mixture was diluted with H₂O (20 mL) and extracted with EtOAc (20 mL*3). The combined organic layers were washed with brine (20 mL), dried over Na₂SO₄, filtered and concentrated under reduced pressure to give a residue. The residue was purified by column chromatography (SiO₂, Petroleum ether/Ethyl acetate=100/1 to 85/15, TLC-Ethyl acetate/Methanol=5/1, Product R_{f}=0.33). 4 (160 mg, 468.15 μmol, 84.08% yield, 99% purity) was obtained as a yellow solid.

LCMS: RT=1.278 min, MS cal.: 338.15, [M+H]⁺=338.9

¹H NMR (400 MHz, CHLOROFORM-d) δ=7.18 (d, J=1.2 Hz, 1H), 7.14 (d, J=1.2 Hz, 1H), 5.08 (tt, J=4.4, 7.2 Hz, 1H), 4.77 (br s, 1H), 4.72 (dt, J=6.4, 7.9 Hz, 1H), 4.58 (td, J=6.0, 9.1 Hz, 1H), 3.93 (s, 3H), 3.47 (d, J=4.4 Hz, 2H), 2.78-2.67 (m, 1H), 2.56 (tdd, J=7.2, 9.1, 11.2 Hz, 1H), 1.44 (s, 9H)

### General Procedure for Preparation of Intermediate 5:

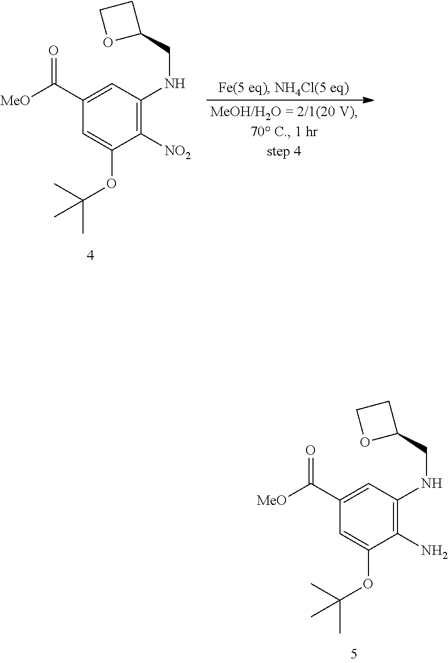 [SMILES: COC(=O)c1cc(NC[C@@H]2CCO2)c([N+](=O)[O-])c(OC(C)(C)C)c1 ; "Fe(5 eq), NH4Cl(5 eq)" ; COC(=O)c1cc(NC[C@@H]2CCO2)c(N)c(OC(C)(C)C)c1]

A mixture of 4 (150 mg, 443.32 μmol, 1 eq) in MeOH (2 mL) was added NH₄Cl (118.57 mg, 2.22 mmol, 5 eq) in H₂O (1 mL), degassed and purged with N₂ for 3 times, and then Fe (123.79 mg, 2.22 mmol, 5 eq) was added to the reaction mixture. After addition, the mixture was stirred at 70° C. for 1 hr under N₂ atmosphere. LC-MS (ET62904-527-p1a1) showed 4 was consumed completely and one main peak with desired mass was detected. The reaction mixture was filtered and concentrated under reduced pressure to give a residue. The residue was diluted with H₂O (20 mL) and extracted with EtOAc (20 mL*3). The combined organic layers were washed with brine (20 mL), dried over Na₂SO₄, filtered and concentrated under reduced pressure to give a residue. 5 (136 mg, crude) was obtained as a colorless oil.

LCMS: RT=1.089 min, MS cal.: 308.17, [M+H]⁺=309.0

### General Procedure for Preparation of Intermediate 6:

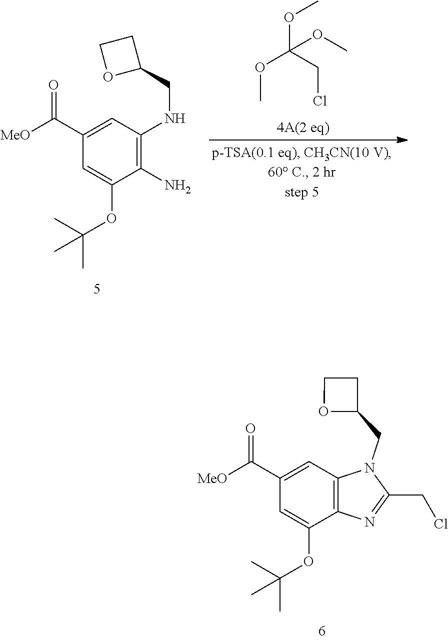

A mixture of 5 (102 mg, 330.77 μmol, 1 eq), 4A (102.27 mg, 661.54 μmol, 89.16 μL, 2 eq), TosOH (5.70 mg, 33.08 μmol, 0.1 eq) in ACN (1 mL) was degassed and purged with N₂ for 3 times, and then the mixture was stirred at 60° C. for 2 hr under N₂ atmosphere. LC-MS showed 5 was consumed completely and one main peak with desired mass was detected. The residue mixture was concentrated under reduced pressure to give a residue. 6 (121 mg, crude) was obtained as a colorless oil.

LCMS: RT=1.151 min, MS cal.: 366.13, [M+H]⁺=366.9

### General Procedure for Preparation of Intermediate 7:

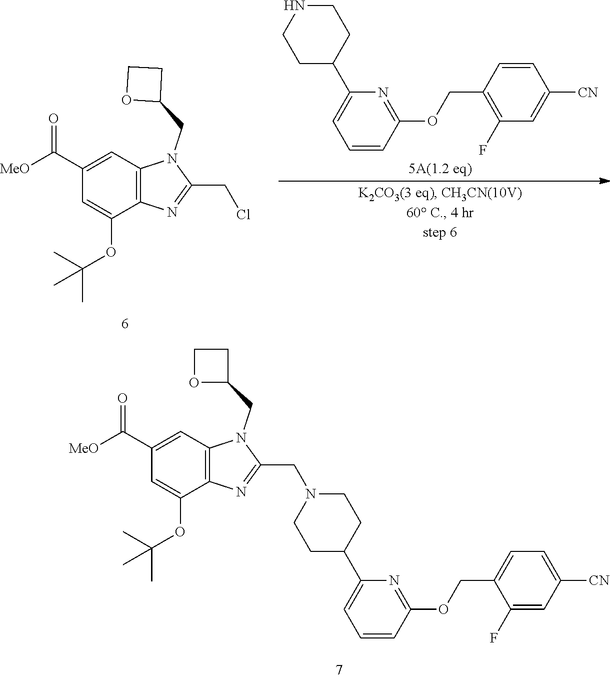 [SMILES: COC(=O)c1cc(OC(C)(C)C)c2nc(CCl)n(C[C@@H]3CCO3)c2c1 ; N#Cc1ccc(COc2cccc(C3CCNCC3)n2)c(F)c1 ; COC(=O)c1cc(OC(C)(C)C)c2nc(CN3CCC(c4cccc(OCc5ccc(C#N)cc5F)n4)CC3)n(C[C@@H]3CCO3)c2c1]

A mixture of 6 (121 mg, 329.85 μmol, 1 eq), 6A (123.24 mg, 395.81 μmol, 1.2 eq), K₂CO₃ (136.76 mg, 989.54 μmol, 3 eq) in ACN (1.2 mL) was degassed and purged with N₂ for 3 times, and then the mixture was stirred at 60° C. for 4 hrs under N₂ atmosphere. LC-MS showed 6 was consumed completely and one main peak with desired mass was detected. The residue mixture was diluted with H₂O (30 mL) and extracted with EtOAc (30 mL*3). The combined organic layers were washed with brine (20 mL), dried over Na₂SO₄, filtered, and concentrated under reduced pressure to give a residue. The residue was purified by column chromatography (SiO₂, Petroleum ether/Ethyl acetate=100/1 to 0/1, TLC-Ethyl acetate/Methanol=0/1, Product R_{f}=0.46). 7 (200 mg, 261.67 μmol, 79.33% yield, 83.96% purity) was obtained as a white solid.

LCMS: RT=1.578 min, MS cal.: 641.30, [M+H]⁺=642.1

¹H NMR (400 MHz, CHLOROFORM-d₄) δ=7.88 (s, 1H), 7.64 (s, 1H), 7.59 (s, 1H), 7.53 (t, J=7.6 Hz, 1H), 7.45 (d, J=8.0 Hz, 1H), 7.38 (d, J=9.2 Hz, 1H), 6.76 (d, J=7.2 Hz, 1H), 6.65 (d, J=8.0 Hz, 1H), 5.51 (s, 2H), 5.27-5.19 (m, 1H), 4.73 (br s, 1H), 4.65-4.58 (m, 1H), 4.41 (td, J=5.6, 9.1 Hz, 1H), 3.97 (br d, J=1.2 Hz, 1H), 3.94 (s, 3H), 3.09-2.90 (m, 2H), 2.79-2.68 (m, 1H), 2.59 (td, J=3.6, 7.4 Hz, 1H), 2.51-2.41 (m, 1H), 2.37-2.23 (m, 2H), 1.89-1.71 (m, 6H), 1.52 (s, 9H)

### General Procedure for Preparation of Compound 109 (8):

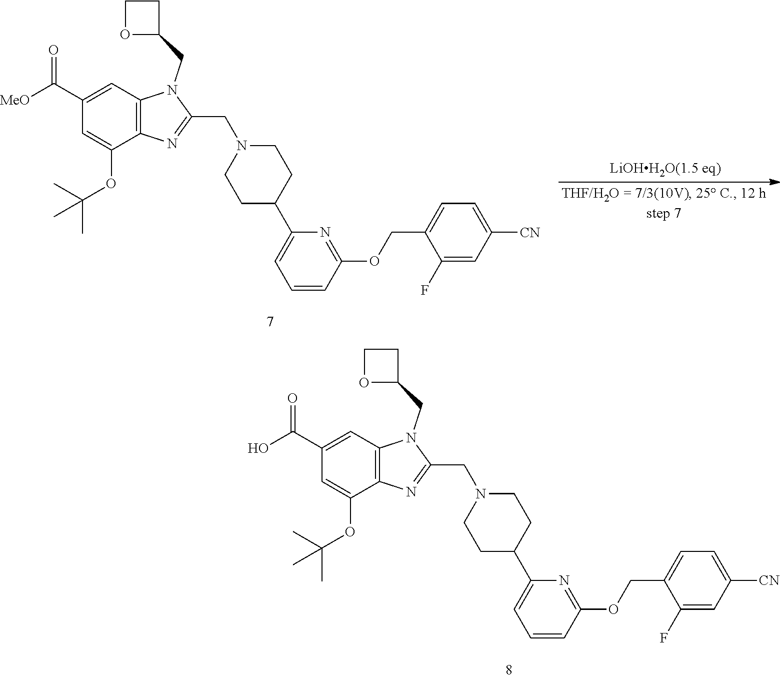 [SMILES: COC(=O)c1cc(OC(C)(C)C)c2nc(CN3CCC(c4cccc(OCc5ccc(C#N)cc5F)n4)CC3)n(C[C@@H]3CCO3)c2c1 ; CC(C)(C)Oc1cc(C(=O)O)cc2c1nc(CN1CCC(c3cccc(OCc4ccc(C#N)cc4F)n3)CC1)n2C[C@@H]1CCO1]

A mixture of 7 (100 mg, 155.83 μmol, 1 eq), LiOH·H₂O (9.81 mg, 233.74 μmol, 1.5 eq) in THF (0.7 mL) and H₂O (0.3 mL) was degassed and purged with N₂ for 3 times, and then the mixture was stirred at 25° C. for 12 hrs under N₂ atmosphere. LC-MS (ET62904-538-p1a1) showed 7 was consumed completely and one main peak with desired mass was detected. The reaction mixture was diluted with ACN 0.5 mL. The liquid was purified by prep-HPLC (column: Waters Xbridge Prep OBD C₁₈ 150*40 mm*10 um; mobile phase: [H₂O (10 mM NH₄HCO₃)-ACN]; gradient: 20%-70% B over 8.0 min). (S)-4-(tert-Butoxy)-2-((4-(6-((4-cyano-2-fluorobenzyl)oxy)pyridin-2-yl)piperidin-1-yl)methyl)-1-(oxetan-2-ylmethyl)-1H-benzo[d]imidazole-6-carboxylic acid (28.91 mg, 45.43 μmol, 29.15% yield, 98.63% purity) was obtained as a white solid.

LCMS: RT=1.588 min, MS cal.: 627.29, [M+H]⁺=628.3

¹H NMR (400 MHz, METHANOL-d₄) δ=8.04 (s, 1H), 7.70-7.64 (m, 1H), 7.63-7.52 (m, 4H), 6.85 (d, J=7.2 Hz, 1H), 6.69 (d, J=8.0 Hz, 1H), 5.52 (s, 2H), 5.31-5.23 (m, 1H), 4.83 (br s, 1H), 4.73-4.61 (m, 2H), 4.49 (td, J=6.0, 9.1 Hz, 1H), 4.13-4.07 (m, 1H), 4.03-3.97 (m, 1H), 3.13 (br d, J=11.2 Hz, 1H), 3.02 (br d, J=12.0 Hz, 1H), 2.87-2.76 (m, 1H), 2.72-2.62 (m, 1H), 2.59-2.50 (m, 1H), 2.49-2.35 (m, 2H), 1.91-1.80 (m, 4H), 1.48 (s, 9H)

### Example A220—Preparation of Compound 108. (S)-2-((4-(6-((4-Cyano-2-fluorobenzyl)oxy)pyridin-2-yl)piperidin-1-yl)methyl)-4-(methoxy-d3)-1-(oxetan-2-ylmethyl)-1H-benzo[d]imidazole-6-carboxylic acid (Compound 108) (5)

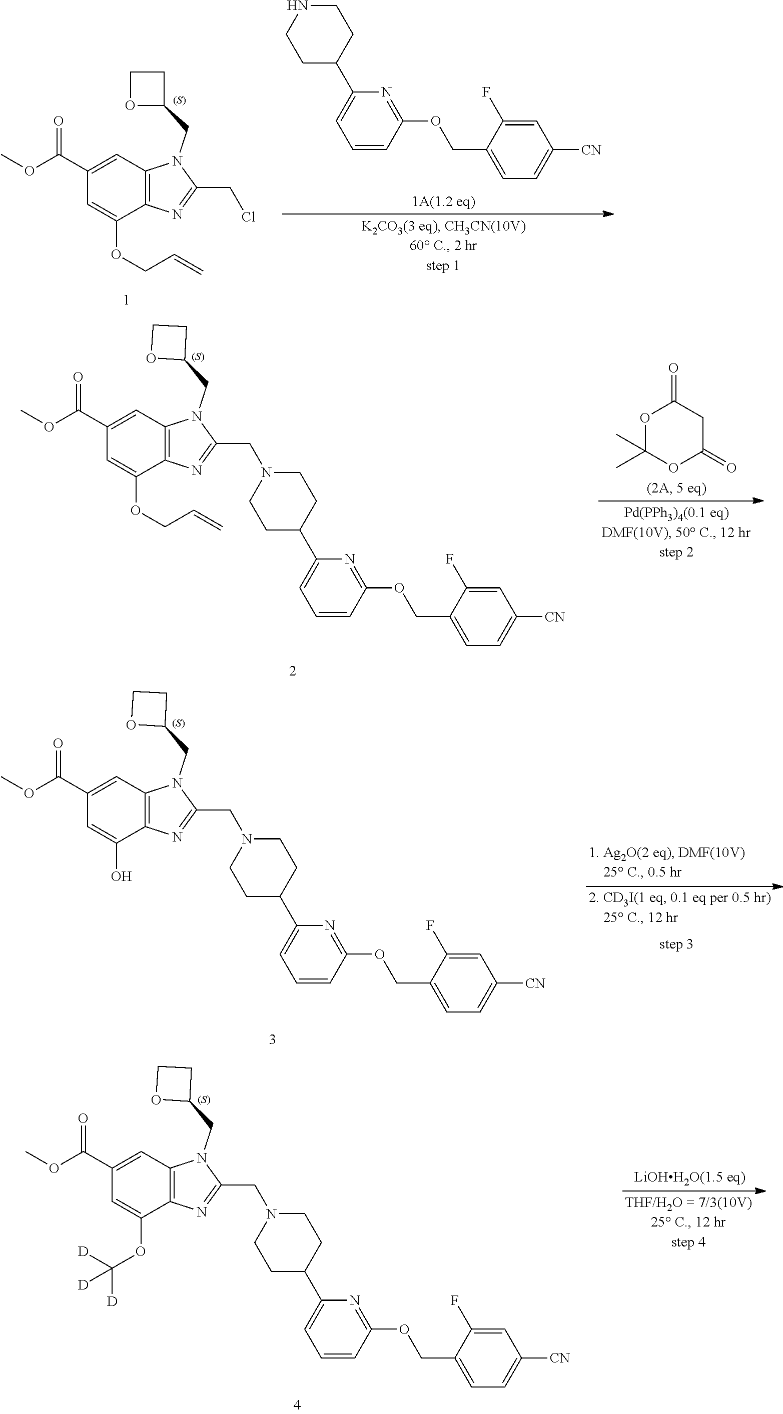

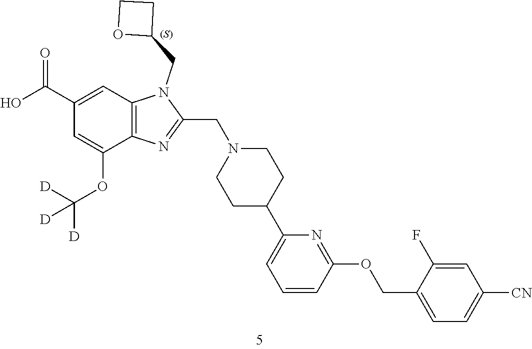

### General Procedure for Preparation of Intermediate 2:

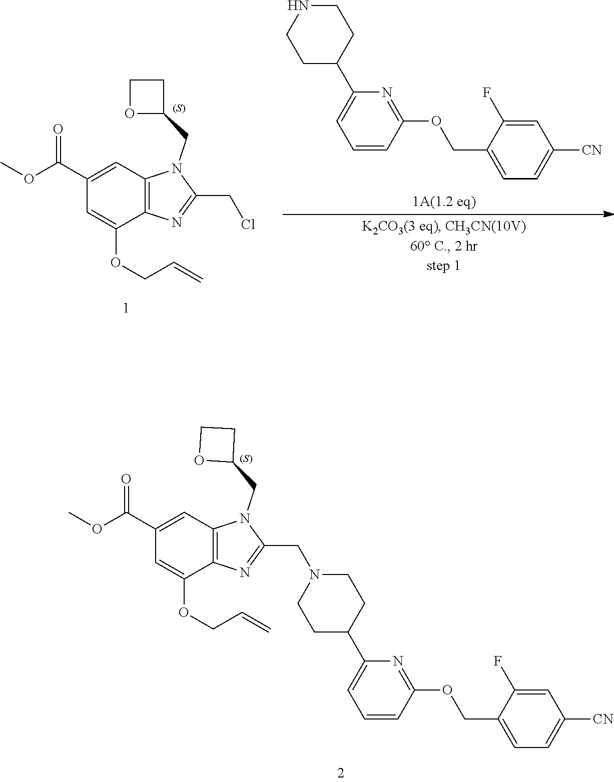

A mixture of 1 (1.2 g, 3.42 mmol, 1 eq), 1A (1.28 g, 4.10 mmol, 1.2 eq), K₂CO₃ (1.42 g, 10.26 mmol, 3 eq) in CH₃CN (12 mL) was degassed and purged with N₂ for 3 times, and then the mixture was stirred at 60° C. for 2 hr under N₂ atmosphere. TLC (Petroleum ether/Ethyl acetate=0/1, R_{f}=0.3) indicated 1 was consumed completely. The reaction mixture was diluted with H₂O (20 mL) and extracted with EtOAc (20 mL*4). The organic phase was dried over Na₂SO₄, filtered, and concentrated under reduced pressure to give a residue. The residue was purified by column chromatography (SiO₂, Petroleum ether/Ethyl acetate=50/1 to Ethyl acetate/Methanol=0/1). 2 (1.65 g, 2.64 mmol, 77% yield) was obtained as a white solid.

LCMS: RT=1.526, MS cal.: 625.27, [M+H]⁺=626.1

¹H NMR (400 MHz, CHCl3-d) δ=7.82 (s, 1H), 7.63 (br t, J=7.6 Hz, 1H), 7.53 (t, J=8.0 Hz, 1H), 7.47-7.35 (m, 3H), 6.76 (d, J=7.2 Hz, 1H), 6.65 (d, J=8.0 Hz, 1H), 6.27-6.13 (m, 1H), 5.51 (s, 2H), 5.47 (dd, J=1.2, 17.2 Hz, 1H), 5.34-5.30 (m, 1H), 5.28-5.18 (m, 1H), 4.87 (d, J=5.6 Hz, 2H), 4.75 (s, 2H), 4.66-4.57 (m, 1H), 4.40 (td, J=5.6, 8.8 Hz, 1H), 4.05-3.92 (m, 5H), 2.95 (s, 2H), 2.79-2.68 (m, 1H), 2.66-2.55 (m, 1H), 2.52-2.41 (m, 1H), 2.27 (d, J=10.8 Hz, 2H), 1.91-1.72 (m, 4H)

### General Procedure for Preparation of Intermediate 3:

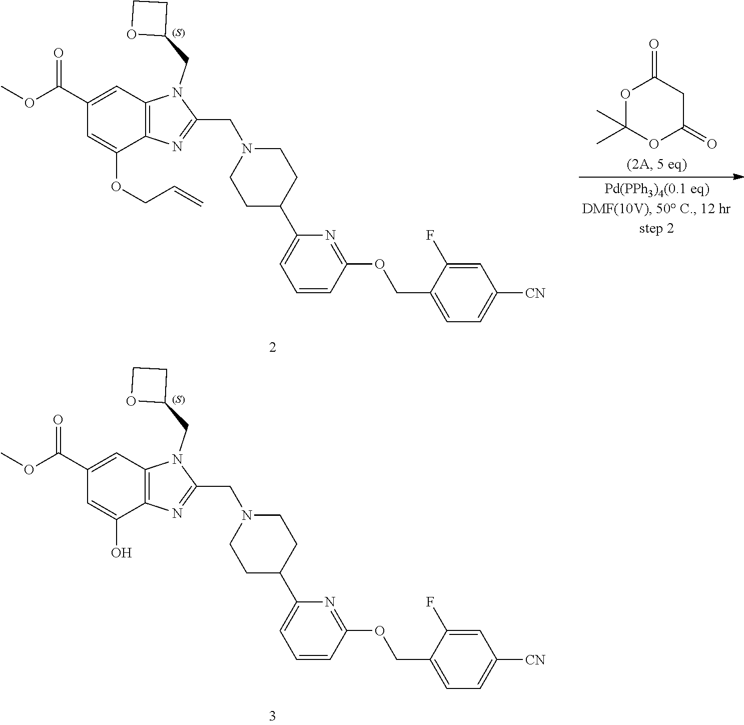

A mixture of 2 (650 mg, 1.04 mmol, 1 eq), 2A (748.63 mg, 5.19 mmol, 5 eq), Pd(PPh₃)₄ (120.05 mg, 103.89 μmol, 0.1 eq) in DMF (7 mL) was degassed and purged with N₂ for 3 times, and then the mixture was stirred at 50° C. for 12 hr under N₂ atmosphere. TLC (Petroleum ether/Ethyl acetate=0/1, R_{f}=0.3) indicated 2 was consumed completely. LCMS (RT=1.376) showed 2 was consumed completely. The reaction mixture was diluted with H₂O (10 mL) and extracted with EtOAc (10 mL*5). The organic phase was washed with brine (10 mL*2), dried over Na₂SO₄, filtered, and concentrated under reduced pressure to give a residue. The residue was purified by column chromatography (SiO₂, Petroleum ether/Ethyl acetate=10/1 to Ethyl acetate/Methanol=0/1). 3 (500 mg, 853.79 μmol, 82% yield) was obtained as an orange solid.

LCMS: RT=2.131 min. MS cal.: 585.2, [M+H]⁺=586.2

¹H NMR (400 MHz, CHCl3-d) δ=7.69-7.59 (m, 2H), 7.59-7.43 (m, 3H), 7.36 (d, J=9.2 Hz, 1H), 6.82-6.65 (m, 2H), 5.47 (s, 2H), 5.18 (d, J=6.4 Hz, 1H), 4.93 (dd, J=6.8, 15.2 Hz, 1H), 4.68-4.45 (m, 5H), 3.95 (s, 3H), 3.49-3.37 (m, 2H), 2.92-2.72 (m, 4H), 2.61-2.45 (m, 1H), 2.04 (s, 4H)

### General Procedure for Preparation of Intermediate 4:

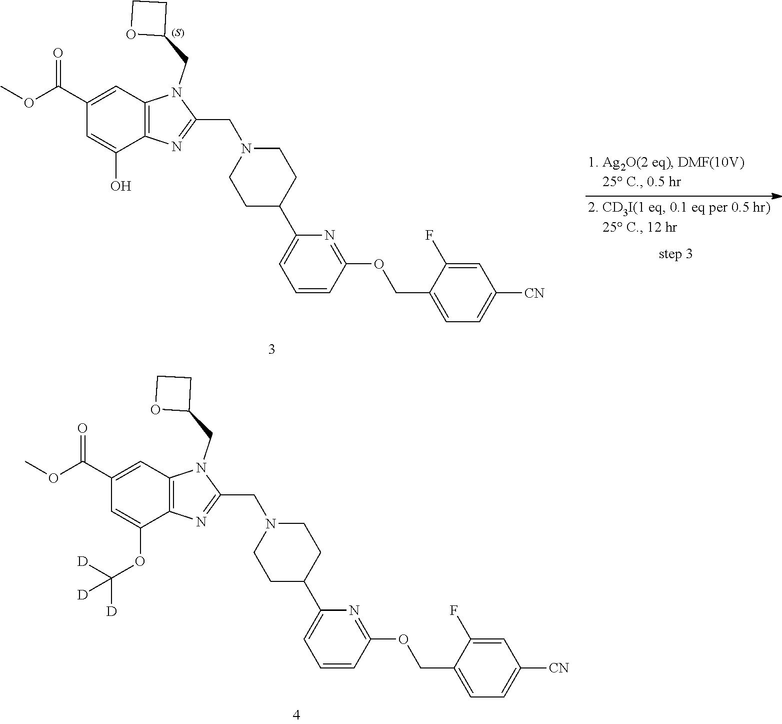

DMF (2 mL) was charged to the 25 mL three-necked round bottom flask, then 3 (200 mg, 341.52 μmol, 1 eq) and Ag₂O (158.28 mg, 683.03 μmol, 2 eq) was added to the mixture at 25° C. CD₃I (48.47 mg, 341.52 μmol, 1 eq) (0.1 eq per 0.5 hr) was added dropwise to the reaction mixture at 25° C. After the addition, the mixture was stirred at 25° C. for 12 hr. LCMS (RT=2.238) showed 3 was consumed completely. The reaction mixture was added to H₂O (6 mL) at 25° C. The mixture was extracted by EtOAc (6 mL*3). Then organic phase was combined and washed by brine (6 mL), dried with Na₂SO₄, filtered. The organic phase was concentrated under reduced pressure at 35° C. to give a residue. The residue was purified by column chromatography (SiO₂, Petroleum ether/Ethyl acetate=100/1 to Ethyl acetate/Methanol=0/1). 4 (80 mg, 132.74 μmol, 39% yield) was obtained as a yellow oil.

LCMS: RT=1.423 min, MS cal.: 602.3; 603.3, [M+H]⁺=603.1

### General Procedure for Preparation of Compound 108 (5):

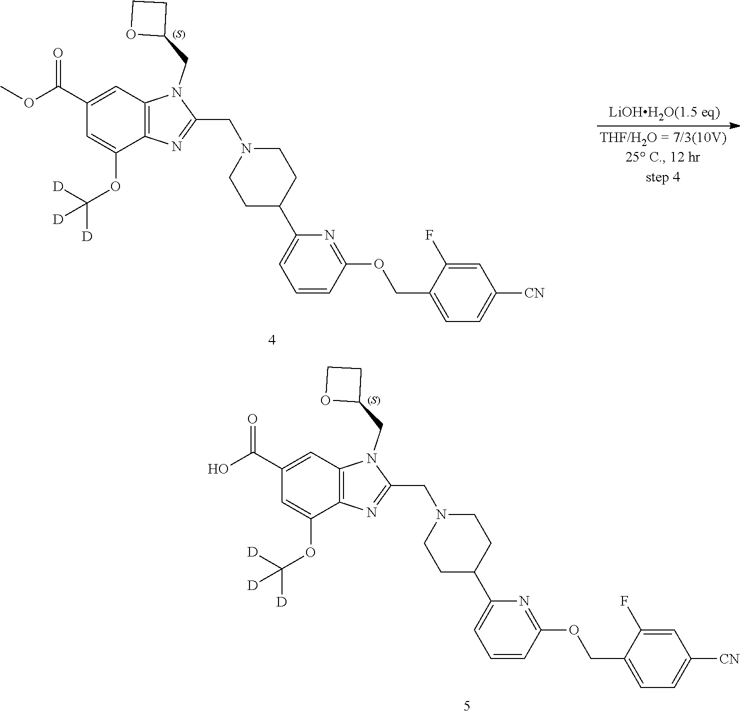

A mixture of 4 (70 mg, 116.15 μmol, 1 eq), LiOH·H₂O (7.31 mg, 174.22 μmol, 1.5 eq) in THF (0.7 mL) and H₂O (0.3 mL) was degassed and purged with N₂ for 3 times, and then the mixture was stirred at 25° C. for 12 hr under N₂ atmosphere. LCMS (reactant RT=2.238, product RT=1.495) showed 8% of Reactant 1 remained and 73% of desired Compound was detected. The reaction mixture was filtered. The filtrate was purified by prep-HPLC (column: Waters Xbridge Prep OBD C₁₈ 150*40 mm*10 um; mobile phase: [H₂O (10 mM NH₄HCO₃)-ACN]; gradient: 15%-65% B over 8.0 min). (S)-2-((4-(6-((4-Cyano-2-fluorobenzyl)oxy)pyridin-2-yl)piperidin-1-yl)methyl)-4-(methoxy-d3)-1-(oxetan-2-ylmethyl)-1H-benzo[d]imidazole-6-carboxylic acid (24.41 mg, 40.79 μmol, 35% yield, 98.36% purity) was obtained as a white solid.

LCMS: RT=2.281 min, MS cal.: 588.3; 589.3, [M+H]⁺=589.3

¹H NMR (400 MHz, DMSO-d6) δ=7.92-7.86 (m, 2H), 7.70 (d, J=3.6 Hz, 2H), 7.64 (t, J=7.6 Hz, 1H), 7.25 (d, J=1.2 Hz, 1H), 6.88 (d, J=7.2 Hz, 1H), 6.72 (d, J=8.4 Hz, 1H), 5.47 (s, 2H), 5.09 (dd, J=2.4, 6.8 Hz, 1H), 4.81-4.71 (m, 1H), 4.67-4.59 (m, 1H), 4.53-4.43 (m, 1H), 4.40-4.31 (m, 1H), 3.90 (d, J=13.2 Hz, 1H), 3.75 (d, J=13.2 Hz, 1H), 2.96 (d, J=10.8 Hz, 1H), 2.83 (d, J=11.2 Hz, 1H), 2.73-2.65 (m, 1H), 2.62-2.55 (m, 1H), 2.42 (dd, J=8.8, 10.8 Hz, 1H), 2.25-2.11 (m, 2H), 1.80-1.61 (m, 4H)

### Example A221—Preparation of Compound 106. 2-((4-(6-((4-Cyano-2-fluorobenzyl)oxy)pyridin-2-yl)piperidin-1-yl)methyl)-4-(difluoromethoxy)-1-(2-methoxyethyl)-1H-benzo[d]imidazole-6-carboxylic acid (Compound 106) (7)

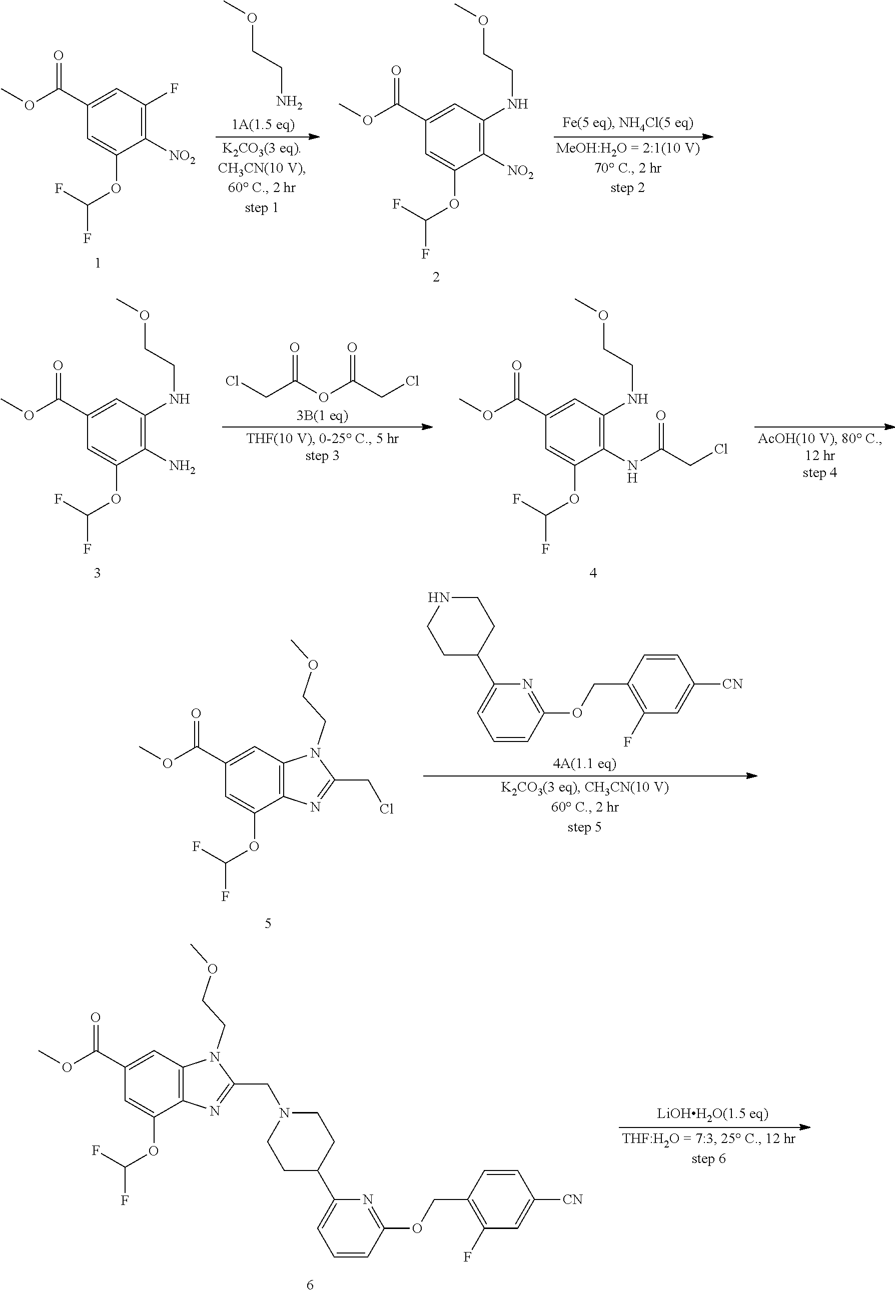

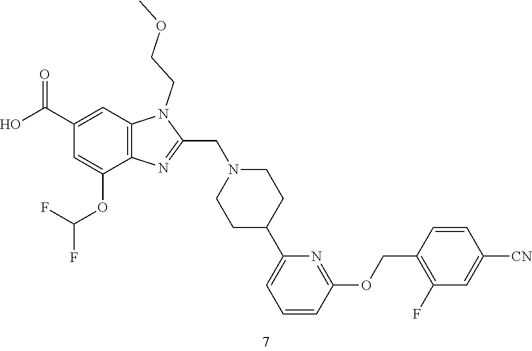 [SMILES: COCCn1c(CN2CCC(c3cccc(OCc4ccc(C#N)cc4F)n3)CC2)nc2c(OC(F)F)cc(C(=O)O)cc21]

### General Procedure for Preparation of Intermediate 2:

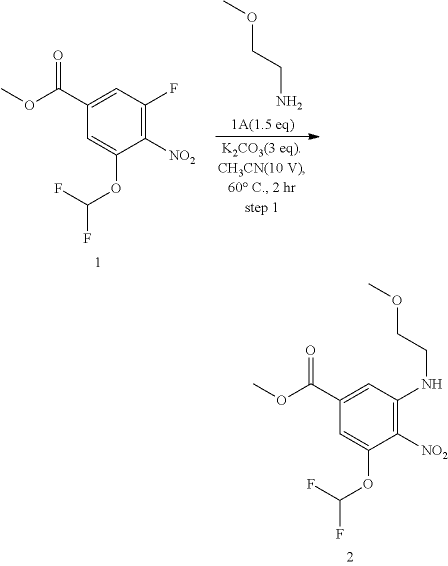

To a solution of 1 (1 g, 3.77 mmol, 1 eq) in CH₃CN (10 mL) was added K₂CO₃ (1.56 g, 11.31 mmol, 3 eq) and 1A (424.92 mg, 5.66 mmol, 491.80 μL, 1.5 eq). The mixture was stirred at 60° C. for 2 hr. TLC (PE/EtOAc-5/1, Rf=0.5) indicated 1 was consumed completely and one new spot formed. The reaction was clean according to TLC. The reaction mixture was diluted with H₂O 20 mL and extracted with EtOAc 60 mL (20 mL*3). The combined organic layers were washed with brine 20 mL, dried over Na₂SO₄, filtered and concentrated under reduced pressure to give a residue. The residue was purified by column chromatography (SiO₂, PE/EtOAc=I/O to 5/1). 2 (1.12 g, 3.46 mmol, 91.80% yield) was obtained as an orange solid.

LCMS: RT=0.479 min, MS cal.: 320.1/321.1, [M+H]⁺=321.0/322.0

¹HNMR (400 MHz, CHCl3-d) δ=7.39 (d, J=1.6 Hz, 1H), 7.19-7.15 (m, 1H), 6.76 (t, J=73.2 Hz, 1H), 6.31-6.18 (m, 1H), 3.95 (s, 3H), 3.67-3.62 (m, 2H), 3.45 (t, J=5.2 Hz, 2H), 3.42 (s, 3H)

### General Procedure for Preparation of Intermediate 3:

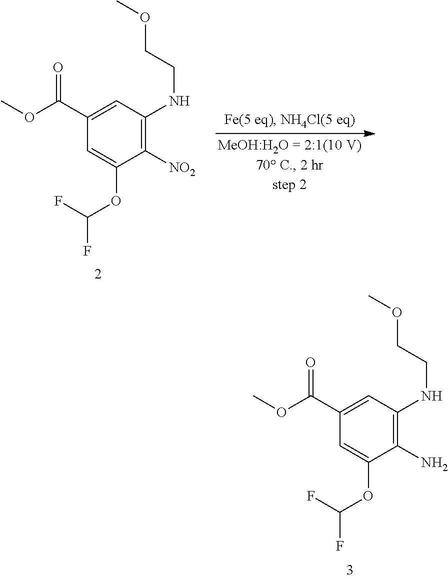 [SMILES: COCCNc1cc(C(=O)OC)cc(OC(F)F)c1[N+](=O)[O-] ; COCCNc1cc(C(=O)OC)cc(OC(F)F)c1N]

Equip a 100 mL one-necked round bottom flask, thermometer, N₂ balloon. MeOH (10 mL) and H₂O (5 mL) was charged to the 100 mL one-necked round bottom flask, then 2 (500 mg, 1.56 mmol, 1 eq) and NH₄Cl (417.58 mg, 7.81 mmol, 5 eq) was added to the mixture at 25° C. Fe (435.95 mg, 7.81 mmol, 5 eq) was added dropwise to the reaction mixture at 25° C. After the addition, the mixture was stirred at 70° C. for 2 hr. TLC (PE/EtOAc-3/1, Rf=0.5) indicated 2 was consumed completely and one new spot formed. The reaction mixture was filtered and the filter cake was washed with MeOH. The residue was diluted with H₂O 10 mL and extracted with EtOAc 20 mL (10 mL*2). The combined organic layers were washed with brine 10 mL, dried over Na₂SO₄, filtered and concentrated under reduced pressure to give a residue. 3 (440 mg, 1.33 mmol, 85.44% yield) was obtained as a yellow solid.

LCMS: RT=1.030 min, MS cal.: 290.1/291.1, [M+H]⁺=291.0/292.0

¹HNMR (400 MHz, CHCl3-d) δ=7.38 (s, 1H), 7.31 (d, J=1.6 Hz, 1H), 6.53 (t, J=74 Hz, 1H), 3.88 (s, 3H), 3.71-3.65 (m, 2H), 3.42 (s, 3H), 3.38-3.34 (m, 2H)

### General Procedure for Preparation of Intermediate 4:

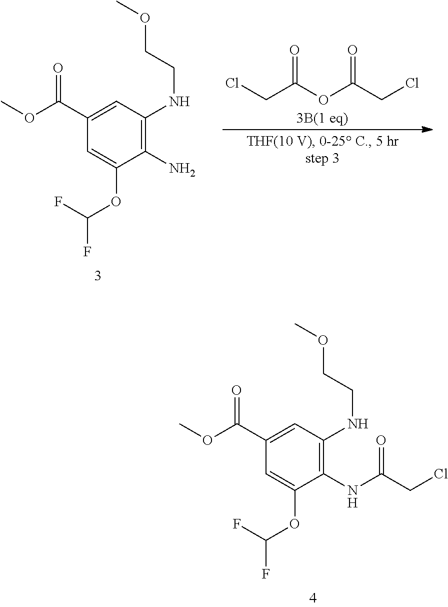 [SMILES: COCCNc1cc(C(=O)OC)cc(OC(F)F)c1N ; O=C(CCl)OC(=O)CCl ; COCCNc1cc(C(=O)OC)cc(OC(F)F)c1NC(=O)CCl]

Equip a 25 mL three-necked round bottom flask, thermometer, N₂ balloon. THF (1.6 mL) was charged to the 25 mL three-necked round bottom flask, then 3 (160 mg, 551.22 μmol, 1 eq) was added to the mixture at 25° C. At 0° C., 3B (94.25 mg, 551.22 μmol, 1 eq) was added dropwise to the reaction mixture at 0° C. After the addition, the mixture was stirred at 0° C. for 2 hr. LCMS showed 22% of 3 remained. Several new peaks were shown on LCMS and 40% of 4 was detected. At 0° C. (inner temperature), Compound 3B (94.25 mg, 551.22 μmol, 1 eq) was added dropwise to the reaction mixture at 0° C. After the addition, the mixture was stirred at 25° C. for 12 hr. LCMS showed 19% of 3 remained. Several new peaks were shown on LCMS and 31% of 4 was detected. The reaction mixture was concentrated under reduced pressure. The crude product 4 (240 mg, crude) as yellow oil was used into the next step without further purification.

LCMS: RT=1.417 min, MS cal.: 366.1/368.1, [M+H]⁺=367.1/369.0

### General Procedure for Preparation of Intermediate 5:

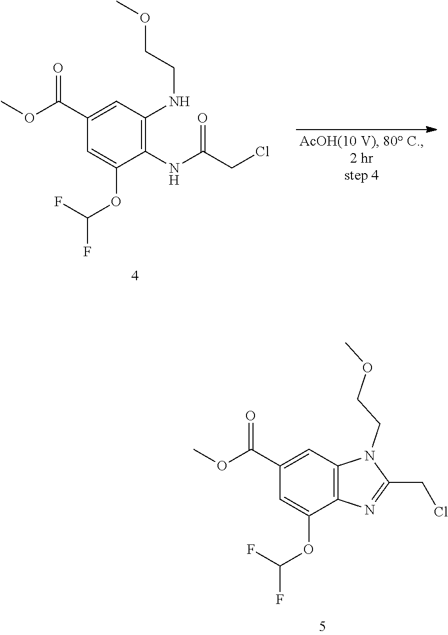 [SMILES: COCCNc1cc(C(=O)OC)cc(OC(F)F)c1NC(=O)CCl ; COCCn1c(CCl)nc2c(OC(F)F)cc(C(=O)OC)cc21]

To a solution of 4 (240 mg, 654.41 μmol, 1 eq) in AcOH (2 mL). The mixture was stirred at 80° C. for 2 hr. LCMS showed 4 was consumed completely and one main peak with desired mass was detected. The reaction mixture was concentrated under reduced pressure. The crude product 5 (240 mg, crude) as yellow oil was used into the next step without further purification.

LCMS: RT=1.587 min, MS cal.: 348.1/350.1, [M+H]⁺=349.0/351.0

### General Procedure for Preparation of Intermediate 6:

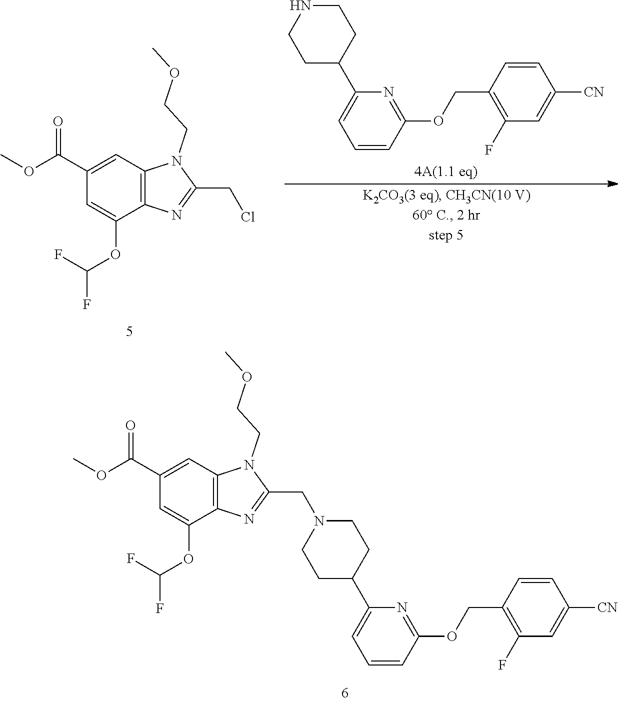 [SMILES: COCCn1c(CCl)nc2c(OC(F)F)cc(C(=O)OC)cc21 ; N#Cc1ccc(COc2cccc(C3CCNCC3)n2)c(F)c1 ; COCCn1c(CN2CCC(c3cccc(OCc4ccc(C#N)cc4F)n3)CC2)nc2c(OC(F)F)cc(C(=O)OC)cc21]

To a solution of 5 (240 mg, 688.21 μmol, 1 eq) in CH₃CN (2.4 mL) was added K₂CO₃ (285.34 mg, 2.06 mmol, 3 eq) and 4A (235.70 mg, 757.03 μmol, 1.1 eq). The mixture was stirred at 60° C. for 2 hr. LCMS showed 5 was consumed completely and one main peak with desired mass was detected. The reaction mixture was diluted with H₂O 5 mL and extracted with EtOAc 10 mL (5 mL*2). The combined organic layers were washed with brine 10 mL, dried over

Na₂SO₄, filtered and concentrated under reduced pressure to give a residue. The residue was purified by column chromatography (SiO₂, PE/EtOAc=1/0 to 1/1). 6 (110 mg, 172.86 μmol, 25.12% yield) was obtained as a white oil.

LCMS: RT=0.486 min, MS cal.: 623.2/624.2, [M+H]⁺=624.2/625.2

### General Procedure for Preparation of Compound 106 (7):

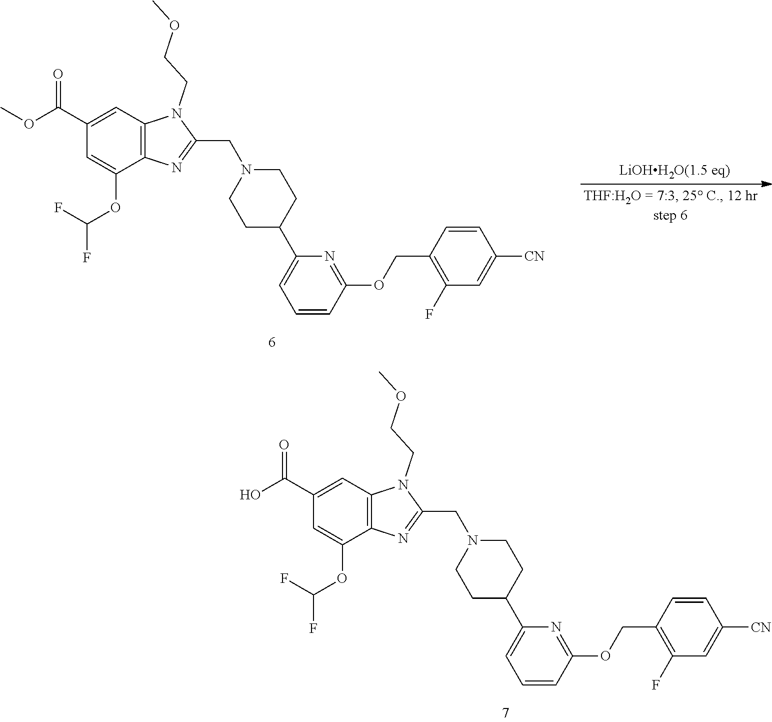 [SMILES: COCCn1c(CN2CCC(c3cccc(OCc4ccc(C#N)cc4F)n3)CC2)nc2c(OC(F)F)cc(C(=O)OC)cc21 ; COCCn1c(CN2CCC(c3cccc(OCc4ccc(C#N)cc4F)n3)CC2)nc2c(OC(F)F)cc(C(=O)O)cc21]

To a solution of 6 (100 mg, 160.35 μmol, 1 eq) in THF (0.7 mL) and H₂O (0.3 mL) was added LiOH H₂O (10.09 mg, 240.53 μmol, 1.5 eq). The mixture was stirred at 25° C. for 12 hr. LCMS showed 6 was consumed completely and one main peak with desired mass was detected. The mixture was diluted with H₂O 1 mL. The residue was purified by prep-HPLC (neutral condition: column: Waters Xbridge Prep OBD C18 150*40 mm*10 um; mobile phase: [H₂O (10 mM NH₄HCO₃)-ACN]; gradient: 25%-65% B over 8.0 min). 2-((4-(6-((4-Cyano-2-fluorobenzyl)oxy)pyridin-2-yl)piperidin-1-yl)methyl)-4-(difluoromethoxy)-1-(2-methoxyethyl)-1H-benzo[d]imidazole-6-carboxylic acid (26.79 mg, 43.51 μmol, 27.13% yield) was obtained as a white solid.

LCMS: RT=2.727 min, MS cal.: 609.2/610.2, [M+H]⁺=610.3/611.3

HPLC: RT=11.130 min

¹H NMR (400 MHz, DMSO-d₆) δ=8.09 (br s, 1H), 7.92-7.84 (m, 1H), 7.73-7.60 (m, 3H), 7.61 (t, J=74 Hz, 1H), 7.51 (s, 1H), 6.88 (d, J=7.2 Hz, 1H), 6.72 (d, J=8.4 Hz, 1H), 5.46 (s, 2H), 4.67-4.61 (m, 2H), 3.85 (s, 2H), 3.80-3.72 (m, 2H), 3.22 (s, 4H), 2.94-2.85 (m, 2H), 2.20 (br t, J=11.2 Hz, 2H), 1.81-1.60 (m, 4H)

### Example A222—Preparation of Compound 105. 2-((4-(6-((4-Cyano-2-fluorobenzyl)oxy)pyridin-2-yl)piperidin-1-yl)methyl)-1-((1-cyanocyclopropyl)methyl)-4-(difluoromethoxy)-1H-benzo[d]imidazole-6-carboxylic acid (Compound 105) (6)

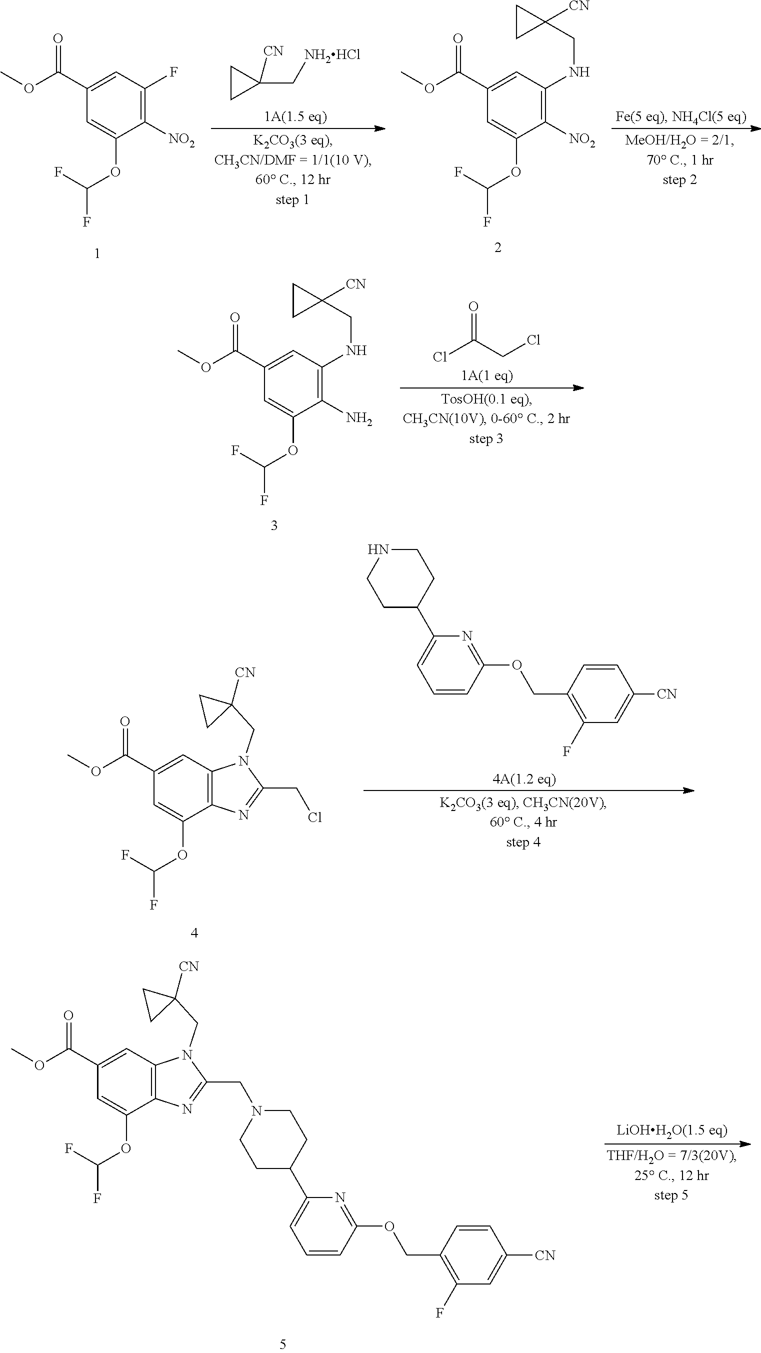

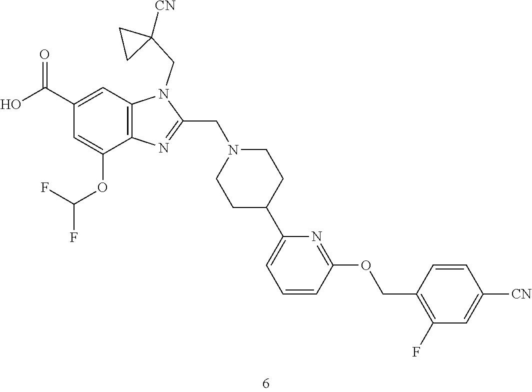 [SMILES: N#Cc1ccc(COc2cccc(C3CCN(Cc4nc5c(OC(F)F)cc(C(=O)O)cc5n4CC4(C#N)CC4)CC3)n2)c(F)c1]

### General Procedure for Preparation of Intermediate 2:

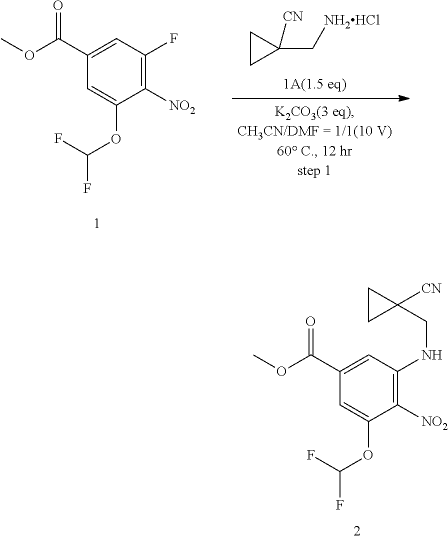

To a solution of 1 (500 mg, 1.89 mmol, 1 eq), 1A (375.06 mg, 2.83 mmol, 1.5 eq) in DMF (3 mL) and CH₃CN (3 mL) was added K₂CO₃ (781.87 mg, 5.66 mmol, 3 eq). The mixture was stirred at 60° C. for 12 hr. TLC indicated 1 was consumed completely and one new spot formed. The residue was diluted with H₂O (20 mL) and extracted with EtOAc 30 mL (10 mL*3). The combined organic layers were washed with brine (20 mL), dried over Na₂SO₄ filtered and concentrated under reduced pressure to give a residue. The residue was purified by column chromatography (SiO₂, Petroleum ether/Ethyl acetate=5/1 to 3/1). 2 (600 mg, 1.76 mmol, 93.23% yield) was obtained as an orange oil.

¹H NMR (400 MHz, DMSO-d₆) δ=7.47 (s, 1H), 7.42 (t, J=72.8 Hz, 1H), 7.09 (s, 1H), 6.93-6.90 (br m, J=6.0 Hz, 1H), 3.89 (s, 3H), 3.46 (d, J=6.0 Hz, 2H), 1.27-1.20 (m, 2H), 1.11-1.05 (m, 2H)

### General Procedure for Preparation of Intermediate 3:

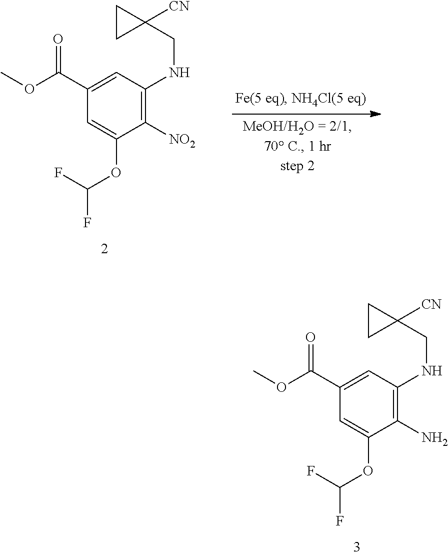 [SMILES: COC(=O)c1cc(NCC2(C#N)CC2)c([N+](=O)[O-])c(OC(F)F)c1 ; COC(=O)c1cc(NCC2(C#N)CC2)c(N)c(OC(F)F)c1]

MeOH (6 mL) was charged to the three-necked round bottom flask, then 2 (600 mg, 1.76 mmol, 1 eq) and NH₄Cl (470.23 mg, 8.79 mmol, 5 eq) in H₂O (3 mL) was added to the mixture at 25° C. At 25° C. (inner temperature), Fe (490.92 mg, 8.79 mmol, 5 eq) was added to the reaction mixture at 25° C. within 2 min. After the addition, the mixture was stirred at 70° C. for 1 hr. LC-MS showed 2 was consumed completely and desired mass was detected. The reaction mixture was filtered with MeOH (20 ml) and the filtrate was concentrated and added to H₂O (20 mL) at 25° C. The mixture was extracted by EtOAc (20 mL). Then organic phase was combined and washed by brine (10 mL), dried with Na₂SO₄, filtered. The organic phase was concentrated under reduced pressure at 40° C. to give a residue. 3 (490 mg, 1.57 mmol, 89.53% yield) was obtained as a yellow oil.

LCMS: RT=1.090 min, MS cal.: 311.1, 312.1, [M+H]⁺=311.9

¹H NMR (400 MHz, DMSO-d₆) δ=7.12 (s, 1H), 7.08 (t, J=74.4 Hz, 1H), 6.98 (d, J=1.6 Hz, 1H), 5.50 (s, 2H), 5.32 (t, J=5.6 Hz, 1H), 3.76 (s, 3H), 3.27 (d, J=5.5 Hz, 2H), 1.28-1.22 (m, 2H), 1.11-1.04 (m, 2H)

### General Procedure for Preparation of Intermediate 4:

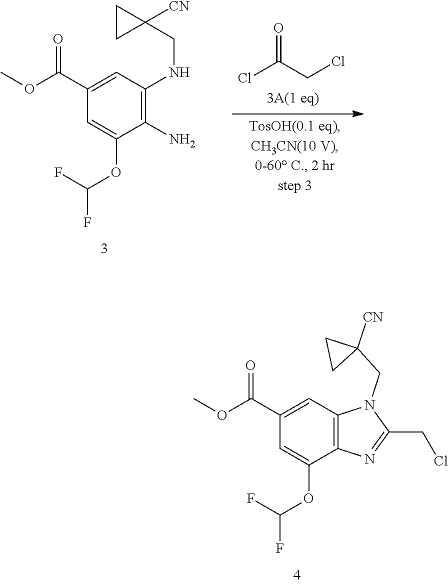 [SMILES: COC(=O)c1cc(NCC2(C#N)CC2)c(N)c(OC(F)F)c1 ; O=C(Cl)CCl ; COC(=O)c1cc(OC(F)F)c2nc(CCl)n(CC3(C#N)CC3)c2c1]

To a solution of 3 (150 mg, 481.88 μmol, 1 eq) in CH₃CN (2 mL) was added TosOH (8.30 mg, 48.19 μmol, 0.1 eq) and 3A (54.42 mg, 481.88 μmol, 38.38 μL, 1 eq) at 0° C. The mixture was stirred at 60° C. for 2 hr. LC-MS showed 3 was consumed completely and desired mass was detected. The reaction mixture was diluted with H₂O (20 mL) and extracted with EtOAc 30 mL (10 mL*3). The combined organic layers were washed with brine (20 mL), dried over Na₂SO₄, filtered and concentrated under reduced pressure to give a residue. The residue was purified by prep-TLC (SiO₂, Petroleum ether/Ethyl acetate=1:1). 4 (50 mg, 135.23 μmol, 28.06% yield) was obtained as a yellow oil.

LCMS: RT=0.470 min, MS cal.: 369.1, 371.1, [M+H]⁺=370.1

### General Procedure for Preparation of Intermediate 5:

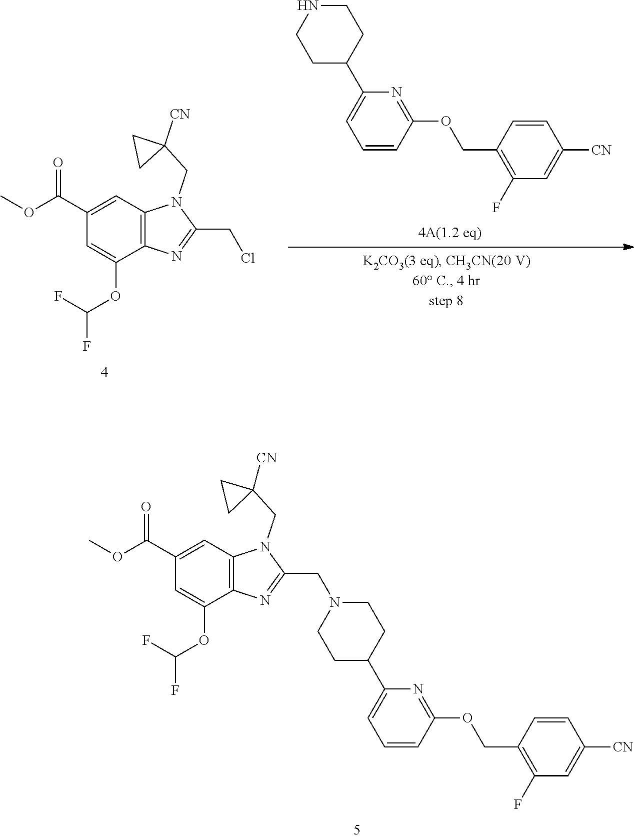 [SMILES: COC(=O)c1cc(OC(F)F)c2nc(CCl)n(CC3(C#N)CC3)c2c1 ; N#Cc1ccc(COc2cccc(C3CCNCC3)n2)c(F)c1 ; COC(=O)c1cc(OC(F)F)c2nc(CN3CCC(c4cccc(OCc5ccc(C#N)cc5F)n4)CC3)n(CC3(C#N)CC3)c2c1]

To a solution of 4 (50 mg, 135.23 μmol, 1 eq), 4A (50.52 mg, 162.27 μmol, 1.2 eq) in CH₃CN (2 mL) was added K₂CO₃ (56.07 mg, 405.68 μmol, 3 eq). The mixture was stirred at 60° C. for 12 hr. LC-MS showed 4 was consumed completely and desired mass was detected. The residue was diluted with H₂O (10 mL) and extracted with EtOAc 15 mL (5 mL*3). The combined organic layers were washed with brine (20 mL), dried over Na₂SO₄ filtered and concentrated under reduced pressure to give a residue. The residue was purified by column chromatography (SiO₂, Petroleum ether/Ethyl acetate=2/1 to 1/1). 5 (45 mg, 69.81 μmol, 51.62% yield) was obtained as a white oil.

LCMS: RT=1.548 min, MS cal.: 644.2, 645.2, [M+H]⁺=645.1

¹H NMR (400 MHz, DMSO-d₆) δ=8.33 (s, 1H), 7.88 (d, J=10.1 Hz, 1H), 7.71-7.67 (m, 2H), 7.67-7.61 (m, 2H), 6.64 (t, J=74 Hz, 1H), 7.57 (s, 1H), 6.88 (d, J=7.3 Hz, 1H), 6.72 (d, J=8.2 Hz, 1H), 5.46 (s, 2H), 4.83 (s, 2H), 3.96-3.88 (m, 5H), 2.93 (br d, J=10.9 Hz, 2H), 2.27-2.14 (m, 2H), 1.80-1.63 (m, 4H), 1.41 (s, 4H)

### General Procedure for Preparation of Compound 105 (6):

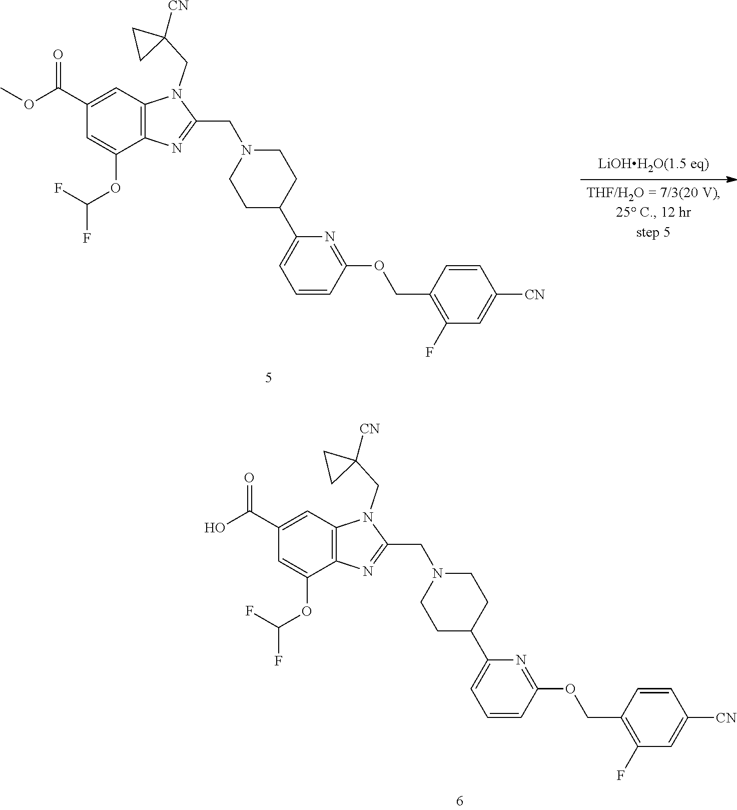 [SMILES: COC(=O)c1cc(OC(F)F)c2nc(CN3CCC(c4cccc(OCc5ccc(C#N)cc5F)n4)CC3)n(CC3(C#N)CC3)c2c1 ; N#Cc1ccc(COc2cccc(C3CCN(Cc4nc5c(OC(F)F)cc(C(=O)O)cc5n4CC4(C#N)CC4)CC3)n2)c(F)c1]

To a solution of 5 (40 mg, 62.05 μmol, 1 eq) in THF (0.7 mL) was added LiOH H₂O (3.91 mg, 93.07 μmol, 1.5 eq) in H₂O (0.3 mL). The mixture was stirred at 25° C. for 12 hr. LC-MS showed 5 was consumed completely and desired mass was detected. The reaction mixture was purified directly. The residue was purified by prep-HPLC (neutral condition; column: Waters Xbridge Prep OBD C18 150*40 mm*10 um; mobile phase: [H₂O (10 mM NH₄HCO₃)-ACN]; gradient: 20%-70% B over 8.0 min). 2-((4-(6-((4-Cyano-2-fluorobenzyl)oxy)pyridin-2-yl)piperidin-1-yl)methyl)-1-((1-cyanocyclopropyl)methyl)-4-(difluoromethoxy)-1H-benzo[d]imidazole-6-carboxylic acid (18.8 mg, 29.81 μmol, 48.05% yield) was obtained as a white solid.

LCMS: RT=2.757 min, MS cal.: 630.2, 631.2, [M+H]⁺=631.3

HPLC: RT=11.251 min, purity: 98.3%

¹H NMR (400 MHz, MeOH-d4) δ=8.23 (s, 1H), 7.72 (s, 1H), 7.70-7.63 (m, 1H), 7.63-7.61 (m, 1H), 7.60-7.57 (m, 1H), 7.56-7.50 (m, 1H), 7.28 (t, J=74.0 Hz, 1H), 6.84 (d, J=7.4 Hz, 1H), 6.69 (d, J=8.2 Hz, 1H), 5.52 (s, 2H), 4.82 (s, 2H), 4.10 (s, 2H), 3.08 (br d, J=10.9 Hz, 2H), 2.72-2.63 (m, 1H), 2.44-2.35 (m, 2H), 1.97-1.82 (m, 4H), 1.52-1.40 (m, 4H)

### Example A223—Preparation of Compound 293. 2-(4-(6-((4-Cyano-2-fluorobenzyl)oxy)pyridin-2-yl)-2-fluorobenzyl)-4-ethoxy-1-methyl-1H-benzo[d]imidazole-6-carboxylic acid (Compound 293) (5)

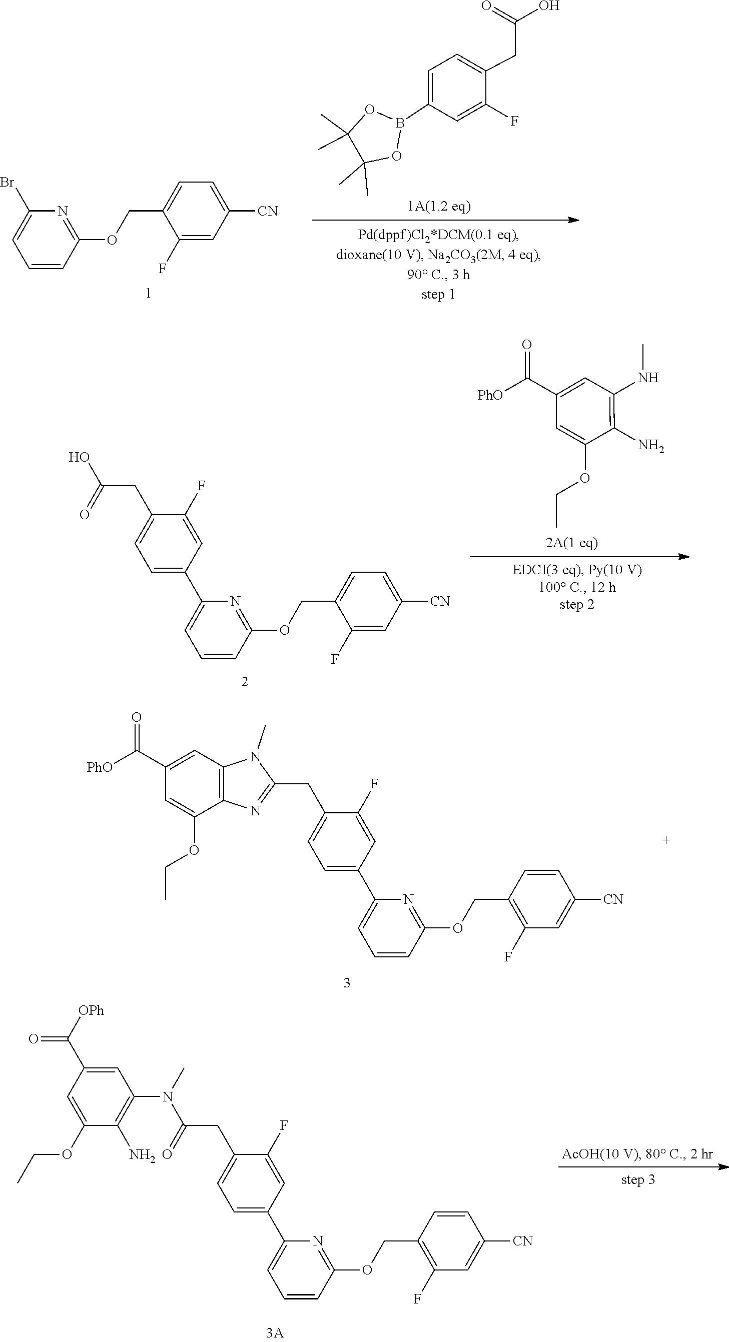 [SMILES: N#Cc1ccc(COc2cccc(Br)n2)c(F)c1 ; CC1(C)OB(c2ccc(CC(=O)O)c(F)c2)OC1(C)C ; N#Cc1ccc(COc2cccc(-c3ccc(CC(=O)O)c(F)c3)n2)c(F)c1 ; CCOc1cc(C(=O)Oc2ccccc2)cc(NC)c1N ; CCOc1cc(C(=O)Oc2ccccc2)cc2c1nc(Cc1ccc(-c3cccc(OCc4ccc(C#N)cc4F)n3)cc1F)n2C ; CCOc1cc(C(=O)Oc2ccccc2)cc(N(C)C(=O)Cc2ccc(-c3cccc(OCc4ccc(C#N)cc4F)n3)cc2F)c1N]

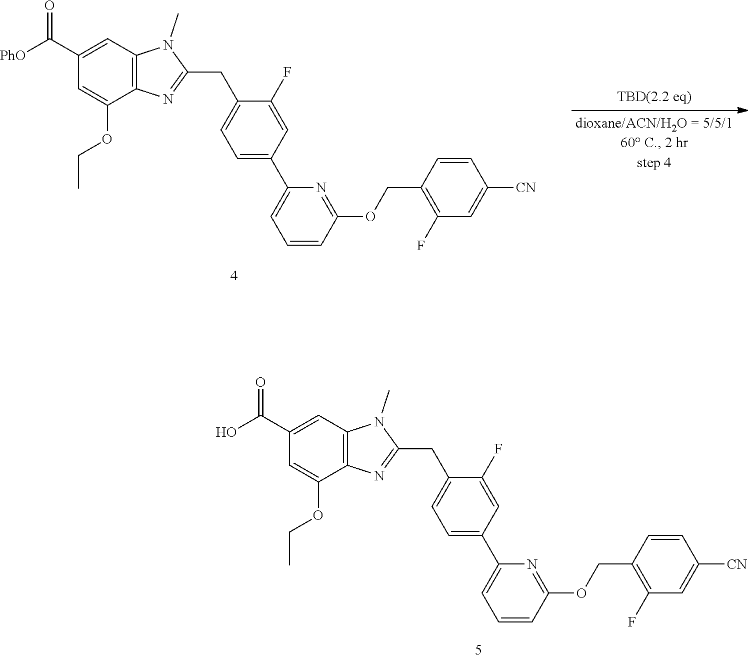 [SMILES: CCOc1cc(C(=O)Oc2ccccc2)cc2c1nc(Cc1ccc(-c3cccc(OCc4ccc(C#N)cc4F)n3)cc1F)n2C ; CCOc1cc(C(=O)O)cc2c1nc(Cc1ccc(-c3cccc(OCc4ccc(C#N)cc4F)n3)cc1F)n2C]

### General Procedure for Preparation of Intermediate 2:

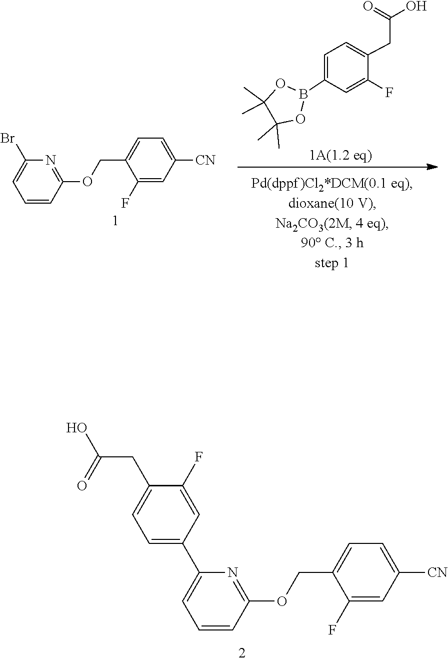 [SMILES: N#Cc1ccc(COc2cccc(Br)n2)c(F)c1 ; CC1(C)OB(c2ccc(CC(=O)O)c(F)c2)OC1(C)C ; N#Cc1ccc(COc2cccc(-c3ccc(CC(=O)O)c(F)c3)n2)c(F)c1]

To a solution of 1 (1 g, 3.26 mmol, 1 eq), 1A (1.09 g, 3.91 mmol, 1.2 eq), Na₂CO₃ (2 M, 6.51 mL, 4 eq) in dioxane (10 mL) was degassed with N₂ for 5 mins, and Pd (dppf) Cl₂·CH₂Cl₂ (265.90 mg, 325.61 μmol, 0.1 eq) was then added to the mixture. The mixture was degassed and purged with N₂ for 3 times, and then the mixture was stirred at 90° C. for 3 hr under N₂ atmosphere. LCMS showed Reactant 1 was consumed completely and desired mass was detected. The reaction mixture was adjusted to pH 5 with 1M FA. Then the reaction mixture was diluted with H₂O 15 mL and extracted with EtOAc (20 mL*3). The combined organic phase was washed with brine (15 mL*2), dried with anhydrous Na₂SO₄, filtered and concentrated in vacuum. The residue was purified by column chromatography (SiO₂, Petroleum ether/Ethyl acetate=1/0 to 0/1). 2 (1.21 g, 3.18 mmol, 97.70% yield) was obtained as a white solid.

LCMS: RT=1.033 min, MS cal: 380.1, [M+H]⁺=380.9

¹H NMR (400 MHz, DMSO-d₆) δ=12.4 (br s, 1H), 7.93 (dd, J=1.2, 10.0 Hz, 1H), 7.90-7.79 (m, 3H), 7.79-7.70 (m, 2H), 7.65 (d, J=7.2 Hz, 1H), 7.50-7.39 (m, 1H), 6.92 (d, J=8.0 Hz, 1H), 5.61 (s, 2H), 3.68 (s, 2H)

### General Procedure for Preparation of Intermediate 3 & 3A:

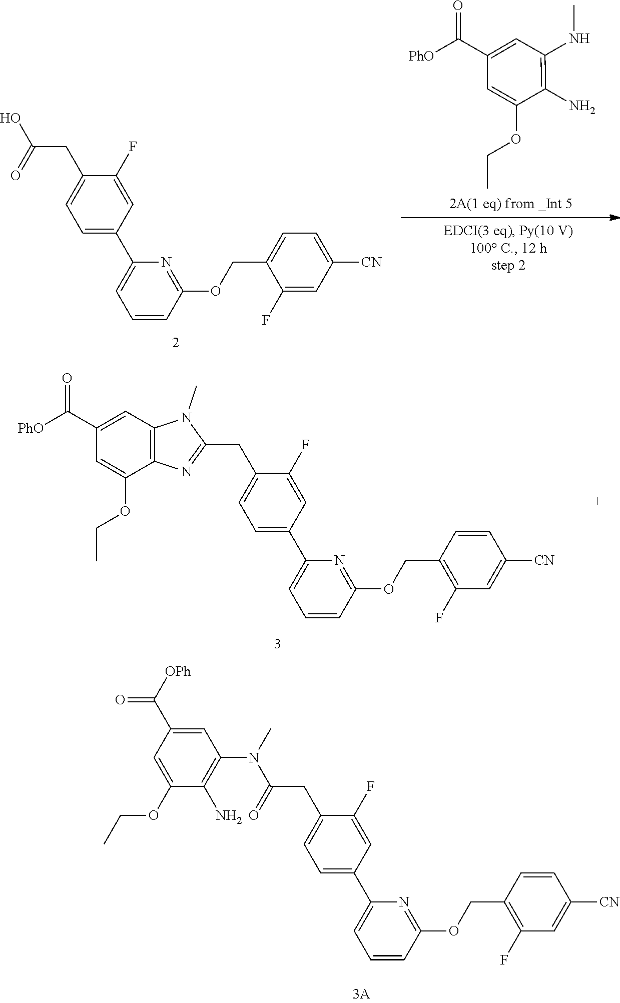 [SMILES: N#Cc1ccc(COc2cccc(-c3ccc(CC(=O)O)c(F)c3)n2)c(F)c1 ; CCOc1cc(C(=O)Oc2ccccc2)cc(NC)c1N ; CCOc1cc(C(=O)Oc2ccccc2)cc2c1nc(Cc1ccc(-c3cccc(OCc4ccc(C#N)cc4F)n3)cc1F)n2C ; CCOc1cc(C(=O)Oc2ccccc2)cc(N(C)C(=O)Cc2ccc(-c3cccc(OCc4ccc(C#N)cc4F)n3)cc2F)c1N]

To a solution of 2 (100 mg, 262.92 μmol, 1 eq) and 2A (75.28 mg, 262.92 μmol, 1 eq) in Py (1 mL) was added EDCI (151.21 mg, 788.76 μmol, 3 eq). The mixture was stirred at 100° C. for 12 hr. Several new peaks were shown on LC-MS and ˜35% of desired compound was detected. The reaction mixture was adjusted to pH 3 with 1M FA. Then 10 mL H₂O was added to the mixture and extracted with EtOAc (5 mL*3). The combined organic phase was washed with brine (5 mL*3), dried with anhydrous Na₂SO₄, filtered and concentrated in vacuum. Without purification. The crude product 3 & 3A (210 mg, crude, ratio: 1:2 in LCMS, a white solid) was obtained as a white solid.

LCMS (3A): RT=2.394 min, MS cal.: 648.2, [M+H]⁺=649.4

### General Procedure for Preparation of Intermediate 4:

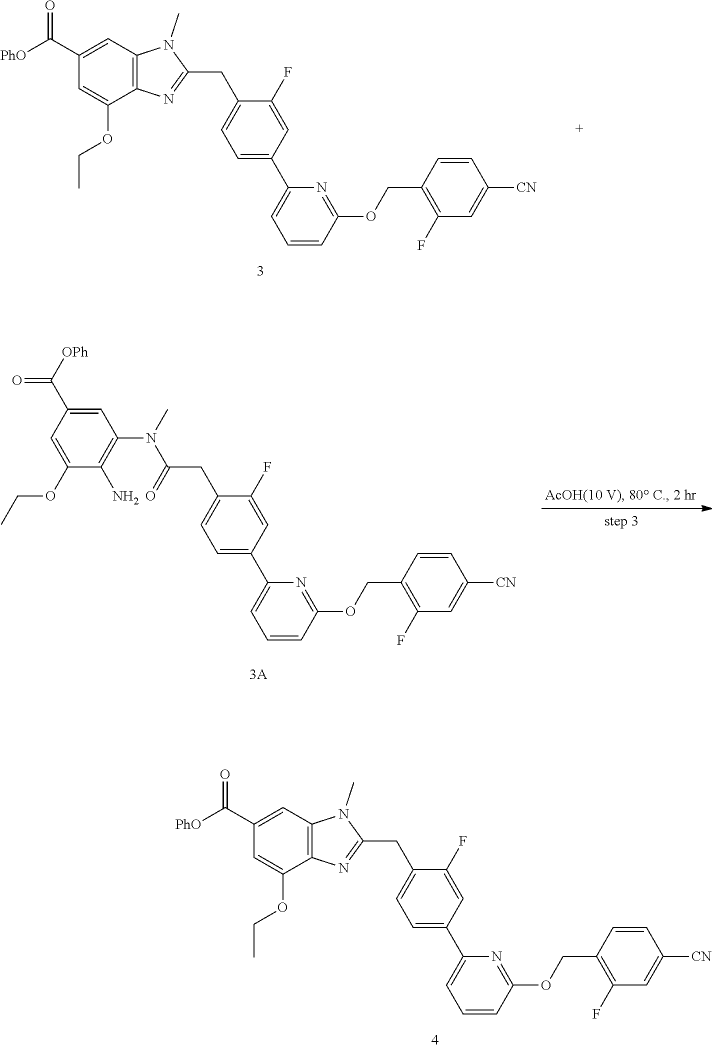 [SMILES: CCOc1cc(C(=O)Oc2ccccc2)cc2c1nc(Cc1ccc(-c3cccc(OCc4ccc(C#N)cc4F)n3)cc1F)n2C ; CCOc1cc(C(=O)Oc2ccccc2)cc(N(C)C(=O)Cc2ccc(-c3cccc(OCc4ccc(C#N)cc4F)n3)cc2F)c1N ; CCOc1cc(C(=O)Oc2ccccc2)cc2c1nc(Cc1ccc(-c3cccc(OCc4ccc(C#N)cc4F)n3)cc1F)n2C]

To a solution of 3 & 3A (210 mg, crude, ratio: 1:2 in LCMS) in AcOH (2.1 mL). The mixture was stirred at 80° C. for 2 hr. LC-MS showed Reactant 1 was consumed completely and desired mass was detected. The reaction mixture was concentrated under reduced pressure to remove CH₃COOH. The reaction mixture was diluted with sat. NaHCO₃ 8 mL and extracted with EtOAc (5 mL*3). The combined organic layers were washed with brine 8 mL dried over anhydrous Na₂SO₄, filtered and concentrated under reduced pressure to give a residue. The residue was purified by column chromatography (SiO₂, Petroleum ether/Ethyl acetate=5/1 to 1/1). Compound 4 (50 mg, 79.28 μmol, 96.67% yield) was obtained as a white solid.

LCMS: RT=2.155 min, MS cal: 630.2, [M+H]⁺=631.4

¹H NMR (400 MHz, DMSO-d₆) δ=8.02 (d, J=1.2 Hz, 1H), 7.98-7.82 (m, 4H), 7.81-7.69 (m, 2H), 7.66 (d, J=7.2 Hz, 1H), 7.53-7.45 (m, 2H), 7.44-7.35 (m, 2H), 7.35-7.23 (m, 3H), 6.92 (d, J=8.0 Hz, 1H), 5.61 (s, 2H), 4.42 (s, 2H), 4.27 (q, J=6.8 Hz, 2H), 3.88 (s, 2H), 1.40 (t, J=6.8 Hz, 4H)

### General Procedure for Preparation of Compound 293 (5):

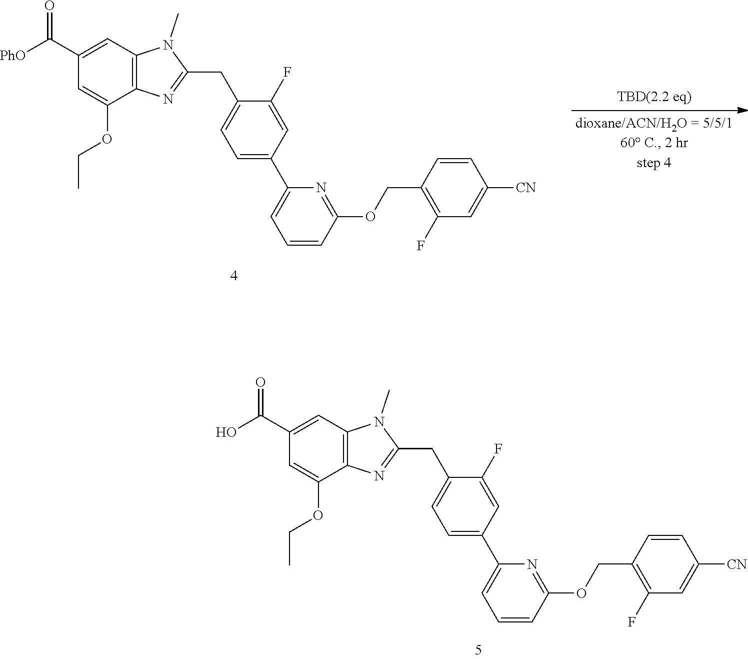 [SMILES: CCOc1cc(C(=O)Oc2ccccc2)cc2c1nc(Cc1ccc(-c3cccc(OCc4ccc(C#N)cc4F)n3)cc1F)n2C ; CCOc1cc(C(=O)O)cc2c1nc(Cc1ccc(-c3cccc(OCc4ccc(C#N)cc4F)n3)cc1F)n2C]

To a solution of 4 (40 mg, 63.43 μmol, 1 eq) in dioxane (0.5 mL), ACN (0.5 mL), H₂O (0.1 mL) was added TBD (19.42 mg, 139.54 μmol, 2.2 eq). The mixture was stirred at 60° C. for 2 hr. LCMS showed Reactant 1 was consumed completely and desired mass was detected. The residue was purified by prep-HPLC (column: Waters Xbridge Prep OBD C18 150*40 mm*10 um; mobile phase: [H₂O (10 mM NH₄HCO₃)-ACN]; gradient: 20%-70% B over 8.0 min). 2-(4-(6-((4-Cyano-2-fluorobenzyl)oxy)pyridin-2-yl)-2-fluorobenzyl)-4-ethoxy-1-methyl-1H-benzo[d]imidazole-6-carboxylic acid (20.77 mg, 37.45 μmol, 59.05% yield, 98.49% purity) was obtained as a white solid.

LCMS: RT=2.531 min, MS cal.: 554.2, [M+H]⁺=555.1

HPLC: RT=12.174 min, purity: 98.49%

¹H NMR (400 MHz, DMSO-d₆) δ=7.98-7.79 (m, 4H), 7.78-7.68 (m, 3H), 7.65 (d, J=7.2 Hz, 1H), 7.34 (t, J=8.0 Hz, 1H), 7.26 (s, 1H), 6.91 (d, J=8.0 Hz, 1H), 5.61 (s, 2H), 4.37 (s, 2H), 4.20 (q, J=6.8 Hz, 2H), 3.79 (s, 3H), 1.38 (t, J=6.8 Hz, 3H)

### Example A224—Preparation of Compound 292. 2-(4-(6-((4-Chloro-2-fluorobenzyl)oxy)pyridin-2-yl)-2-fluorobenzyl)-4-methoxy-1-methyl-1H-benzo[d]imidazole-6-carboxylic acid (Compound 292) (3)

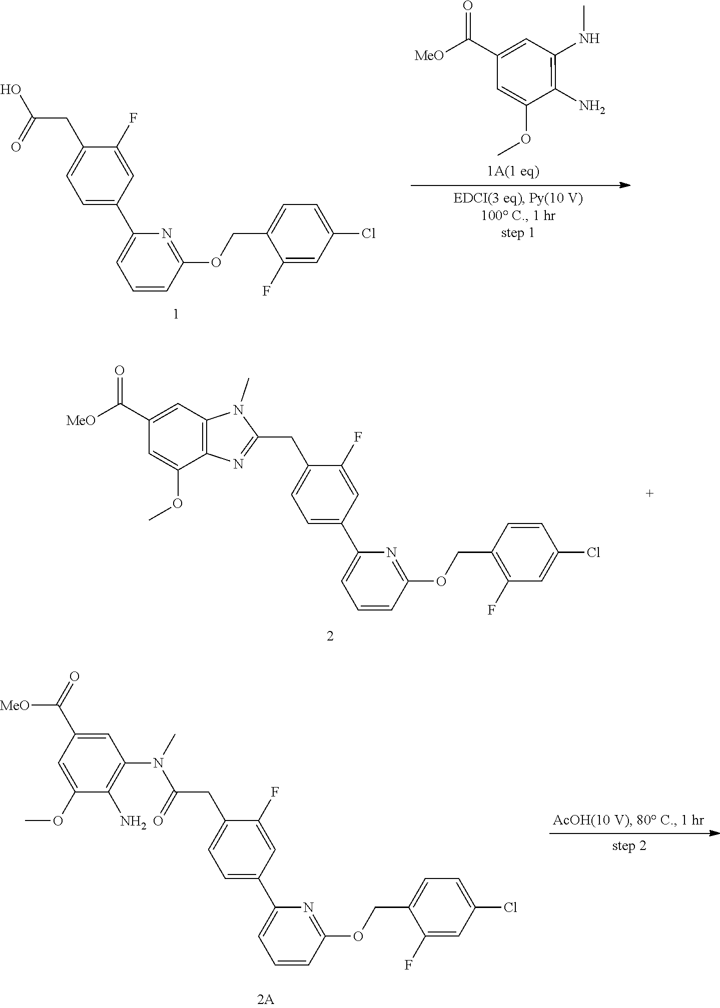 [SMILES: O=C(O)Cc1ccc(-c2cccc(OCc3ccc(Cl)cc3F)n2)cc1F ; CNc1cc(C(=O)OC)cc(OC)c1N ; COC(=O)c1cc(OC)c2nc(Cc3ccc(-c4cccc(OCc5ccc(Cl)cc5F)n4)cc3F)n(C)c2c1 ; COC(=O)c1cc(OC)c(N)c(N(C)C(=O)Cc2ccc(-c3cccc(OCc4ccc(Cl)cc4F)n3)cc2F)c1]

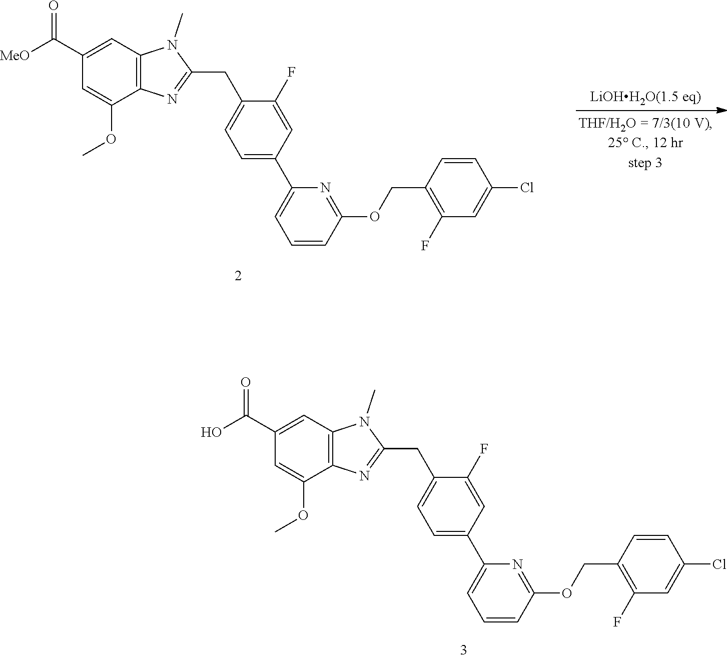 [SMILES: COC(=O)c1cc(OC)c2nc(Cc3ccc(-c4cccc(OCc5ccc(Cl)cc5F)n4)cc3F)n(C)c2c1 ; COc1cc(C(=O)O)cc2c1nc(Cc1ccc(-c3cccc(OCc4ccc(Cl)cc4F)n3)cc1F)n2C]

### General Procedure for Preparation of Intermediate 2 & 2A:

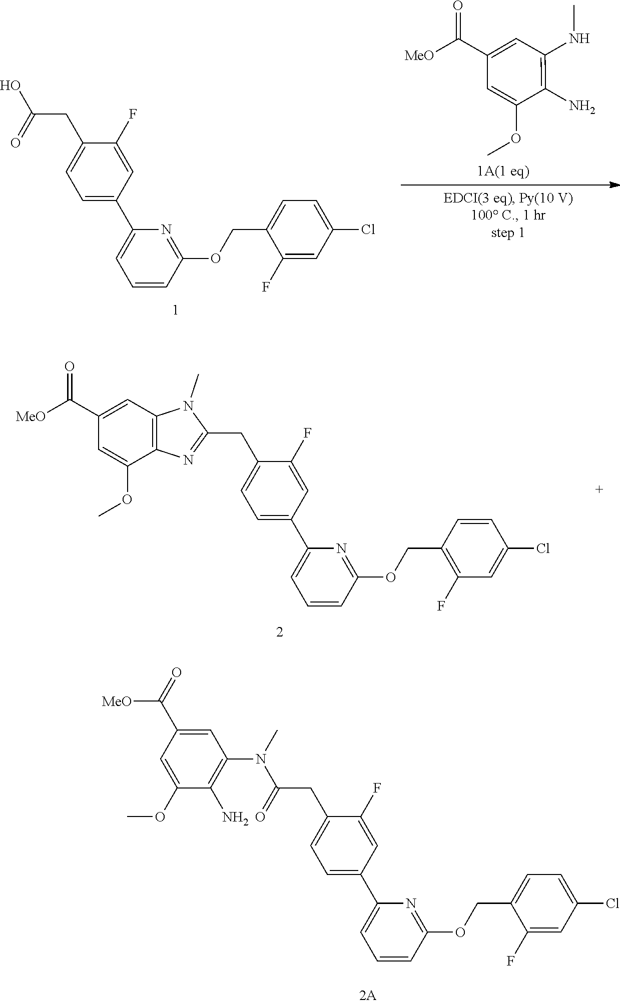 [SMILES: O=C(O)Cc1ccc(-c2cccc(OCc3ccc(Cl)cc3F)n2)cc1F ; CNc1cc(C(=O)OC)cc(OC)c1N ; COC(=O)c1cc(OC)c2nc(Cc3ccc(-c4cccc(OCc5ccc(Cl)cc5F)n4)cc3F)n(C)c2c1 ; COC(=O)c1cc(OC)c(N)c(N(C)C(=O)Cc2ccc(-c3cccc(OCc4ccc(Cl)cc4F)n3)cc2F)c1]

To a solution of 1 (250 mg, 641.39 μmol, 1 eq), 1A (134.84 mg, 641.39 μmol, 1 eq) in Py 3 mL was added EDCI (307.39 mg, 1.60 mmol, 2.5 eq). The mixture was stirred at 100° C. for 1 hr. LC-MS showed 1 was consumed completely and desired mass was detected. The reaction mixture was adjusted with FA (1M) to pH-4. The residue was diluted with H₂O 20 mL and extracted with EtOAc (10 mL*3). The combined organic layers were washed with brine 20 mL, dried over Na₂SO₄ filtered and concentrated under reduced pressure to give residue. 2 & 2A (160 mg, crude) was obtained as a brown oil.

LCMS: RT=2.046 min, MS cal.: 463.14, [M+H]⁺=564.3

### General Procedure for Preparation of Intermediate 2:

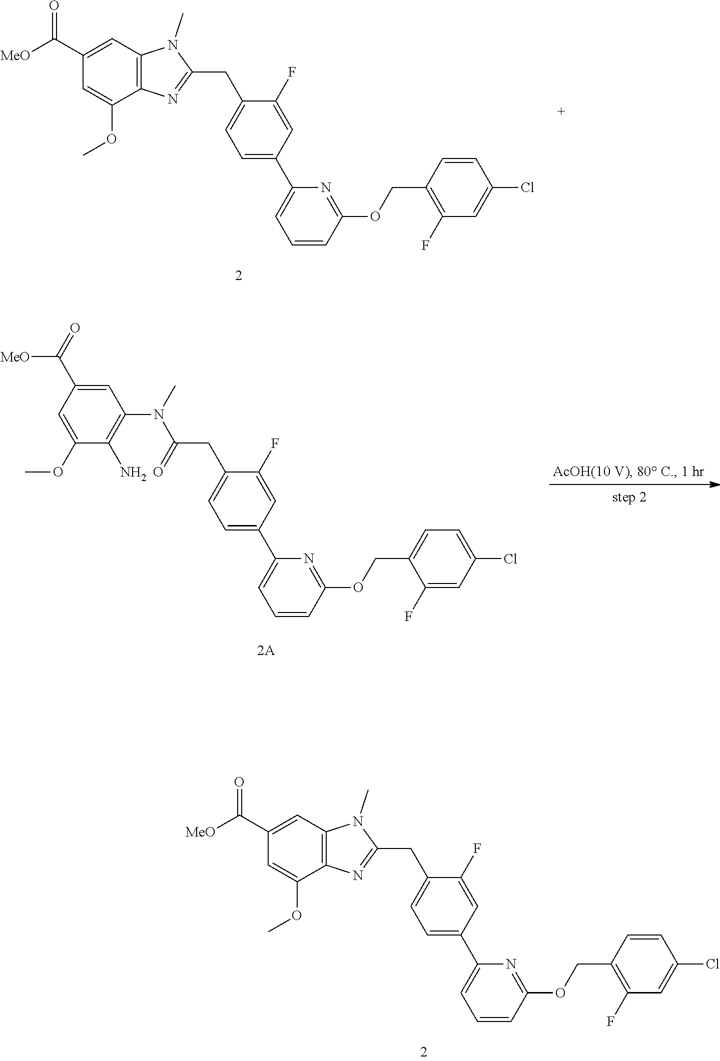 [SMILES: COC(=O)c1cc(OC)c2nc(Cc3ccc(-c4cccc(OCc5ccc(Cl)cc5F)n4)cc3F)n(C)c2c1 ; COC(=O)c1cc(OC)c(N)c(N(C)C(=O)Cc2ccc(-c3cccc(OCc4ccc(Cl)cc4F)n3)cc2F)c1 ; COC(=O)c1cc(OC)c2nc(Cc3ccc(-c4cccc(OCc5ccc(Cl)cc5F)n4)cc3F)n(C)c2c1]

To a solution of 2 & 2A (160 mg, 274.92 μmol, 1 eq) was in AcOH 2 mL. The mixture was stirred at 80° C. for 1 hr. LC-MS showed 2A was consumed completely and desired mass was detected. The reaction mixture was adjusted with NaHCO₃ to pH=8. The residue was diluted with H₂O 10 mL and extracted with EtOAc (5 mL*3). The combined organic layers were washed with brine 10 mL, dried over Na₂SO₄ filtered and concentrated under reduced pressure to give a residue. The residue was purified by column chromatography (SiO₂, Petroleum ether/Ethyl acetate=2/1 to 0/1). 2 (120 mg, 212.77 μmol, 77.40% yield) was obtained as a white solid.

LCMS: RT=2.018 min, MS cal.: 363.14, [M+H]⁺=564.4

¹H NMR (400 MHz, DMSO-d₆) δ=7.94-7.87 (m, 2H), 7.86-7.79 (m, 2H), 7.66-7.58 (m, 2H), 7.49 (dd, J=2.0, 10.0 Hz, 1H), 7.40 (t, J=8.0 Hz, 1H), 7.32 (dd, J=1.6, 8.4 Hz, 1H), 7.26 (d, J=1.2 Hz, 1H), 6.87 (d, J=8.0 Hz, 1H), 5.52 (s, 2H), 4.39 (s, 2H), 3.92 (s, 3H), 3.88 (s, 3H), 3.85 (s, 3H)

### General Procedure for Preparation of Compound 292 (3):

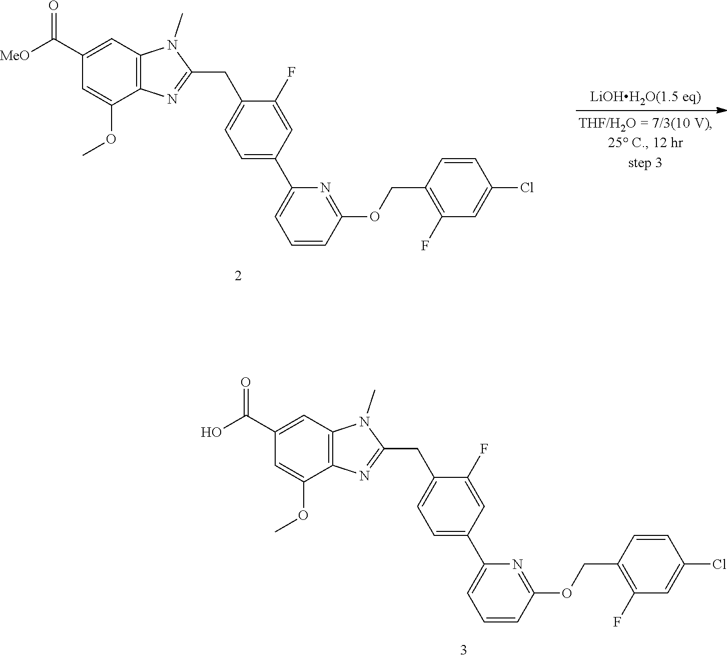 [SMILES: COC(=O)c1cc(OC)c2nc(Cc3ccc(-c4cccc(OCc5ccc(Cl)cc5F)n4)cc3F)n(C)c2c1 ; COc1cc(C(=O)O)cc2c1nc(Cc1ccc(-c3cccc(OCc4ccc(Cl)cc4F)n3)cc1F)n2C]

To a solution of 2 (120 mg, 212.77 μmol, 1 eq) in THF (0.7 mL) was added LiOH H₂O (13.39 mg, 319.16 μmol, 1.5 eq) in H₂O (0.3 mL). The mixture was stirred at 25° C. for 12 hr. LC-MS showed 2 was consumed completely and desired mass was detected. The reaction mixture was purified directly. The reaction was purified by prep-HPLC (neutral condition; column: Waters Xbridge Prep OBD C₁₈ 150*40 mm*10 um; mobile phase: [H₂O (10 mM NH₄HCO₃)-ACN]; gradient: 30%-60% B over 8.0 min). 2-(4-(6-((4-Chloro-2-fluorobenzyl)oxy)pyridin-2-yl)-2-fluorobenzyl)-4-methoxy-1-methyl-1H-benzo[d]imidazole-6-carboxylic acid (29.98 mg, 54.51 μmol, 25.62% yield) was obtained as a white solid.

LCMS: RT=2.803 min, MS cal.: 549.13, [M+H]⁺=550.2

HPLC: RT=11.251 min, purity: 100.0%

¹H NMR (400 MHz, DMSO-d₆) δ=7.95-7.87 (m, 2H), 7.83 (t, J=8.0 Hz, 1H), 7.71 (s, 1H), 7.67-7.58 (m, 2H), 7.49 (d, J=10.0 Hz, 1H), 7.38 (t, J=8.0 Hz, 1H), 7.32 (d, J=8.4 Hz, 1H), 7.28 (s, 1H), 6.87 (d, J=8.4 Hz, 1H), 5.52 (s, 2H), 4.36 (s, 2H), 3.89 (s, 3H), 3.80 (s, 3H)

### Example A225—Preparation of Compound 291. 2-((4-(6-((4-Cyano-2-fluorobenzyl)oxy)pyridin-2-yl)piperidin-1-yl)methyl)-4-(2-hydroxyethoxy)-1-methyl-1H-benzo[d]imidazole-6-carboxylic acid (Compound 291) (4)

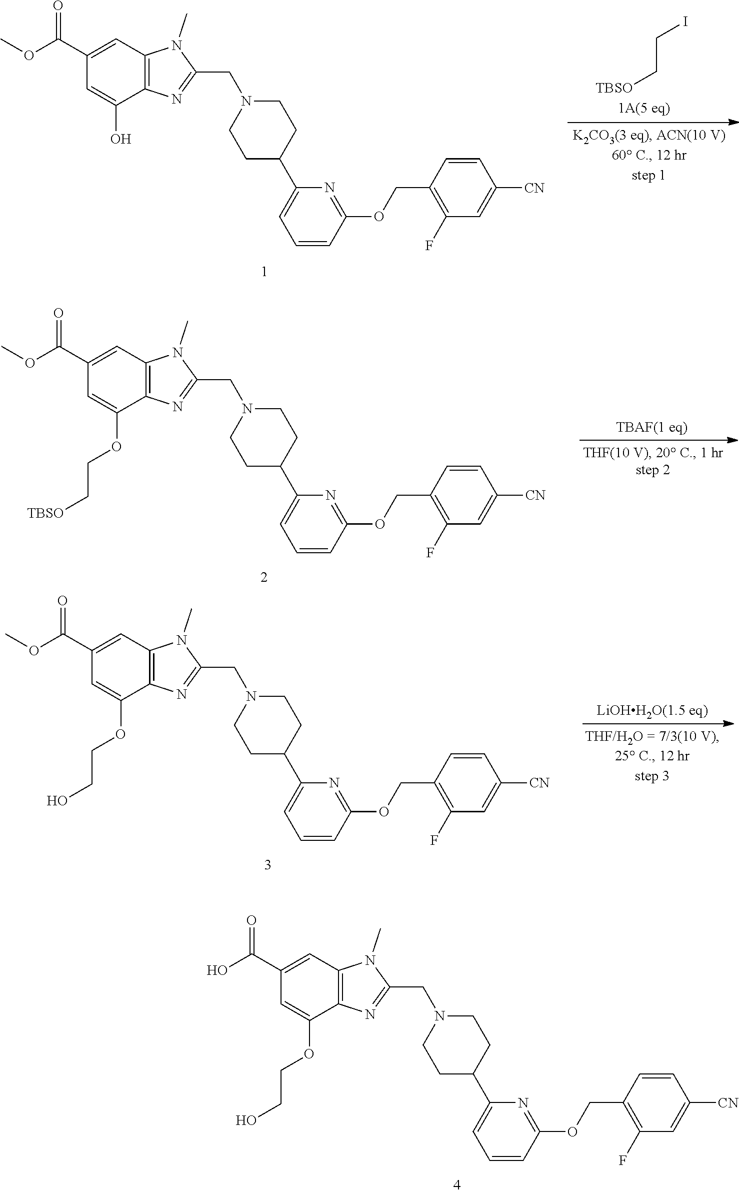 [SMILES: COC(=O)c1cc(O)c2nc(CN3CCC(c4cccc(OCc5ccc(C#N)cc5F)n4)CC3)n(C)c2c1 ; CC(C)(C)[Si](C)(C)OCCI ; COC(=O)c1cc(OCCO[Si](C)(C)C(C)(C)C)c2nc(CN3CCC(c4cccc(OCc5ccc(C#N)cc5F)n4)CC3)n(C)c2c1 ; COC(=O)c1cc(OCCO)c2nc(CN3CCC(c4cccc(OCc5ccc(C#N)cc5F)n4)CC3)n(C)c2c1 ; Cn1c(CN2CCC(c3cccc(OCc4ccc(C#N)cc4F)n3)CC2)nc2c(OCCO)cc(C(=O)O)cc21]

### General Procedure for Preparation of Intermediate 2:

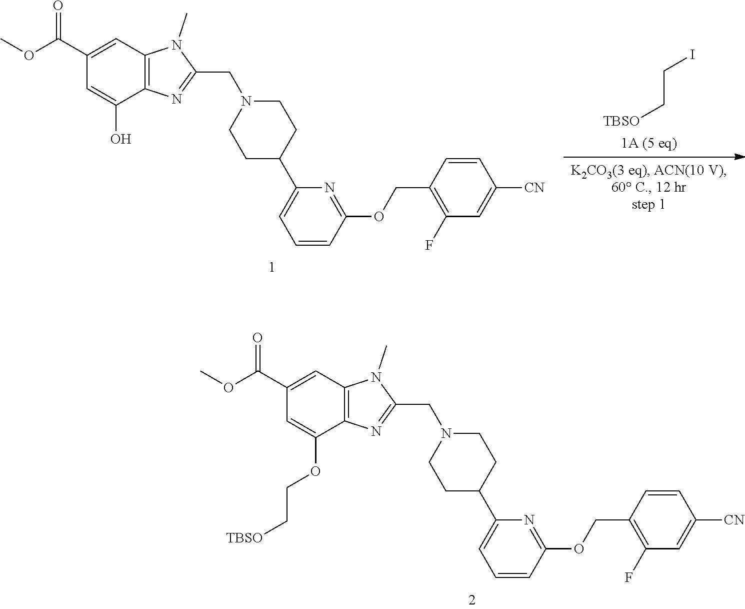 [SMILES: COC(=O)c1cc(O)c2nc(CN3CCC(c4cccc(OCc5ccc(C#N)cc5F)n4)CC3)n(C)c2c1 ; CC(C)(C)[Si](C)(C)OCCI ; COC(=O)c1cc(OCCO[Si](C)(C)C(C)(C)C)c2nc(CN3CCC(c4cccc(OCc5ccc(C#N)cc5F)n4)CC3)n(C)c2c1]

A mixture of 1 (100 mg, 188.84 μmol, 1 eq), 1A (270.25 mg, 944.18 μmol, 5 eq), K₂CO₃ (78.29 mg, 566.51 μmol, 3 eq) in ACN 1 mL was degassed and purged with N₂ for 3 times, and then the mixture was stirred at 60° C. for 12 hrs under N₂ atmosphere. LC-MS showed 1 was consumed completely and one main peak with desired mass was detected. The reaction mixture was diluted with H₂O 50 mL and extracted with EtOAc (5 mL*3). The combined organic layers were washed with brine 50 mL, dried over Na₂SO₄, filtered and concentrated under reduced pressure to give a residue. The residue was purified by column chromatography (SiO₂, Petroleum ether/Ethyl acetate=100/1 to 2/1, TLC-Petroleum ether/Ethyl acetate=2/1, Product R_{f}=0.08). 2 (90 mg, 130.84 μmol, 69.29% yield) was obtained as a brown solid.

LCMS: RT=1.857 min, MS cal.: 687.33, [M+H]⁺=688.2

¹H NMR (400 MHz, CHLOROFORM-d) δ=7.77 (s, 1H), 7.63 (t, J=7.2 Hz, 1H), 7.53 (t, J=7.6 Hz, 1H), 7.47-7.41 (m, 2H), 7.37 (d, J=9.2 Hz, 1H), 6.75 (d, J=7.2 Hz, 1H), 6.64 (d, J=8.0 Hz, 1H), 5.50 (s, 2H), 4.37 (t, J=6.4 Hz, 2H), 4.13-4.10 (m, 2H), 3.96 (s, 6H), 3.88 (s, 2H), 2.95 (d, J=11.2 Hz, 2H), 2.59 (s, 1H), 2.27 (t, J=10.8 Hz, 2H), 1.89-1.72 (m, 4H), 0.90 (s, 9H), 0.10 (s, 6H)

### General Procedure for Preparation of Intermediate 3:

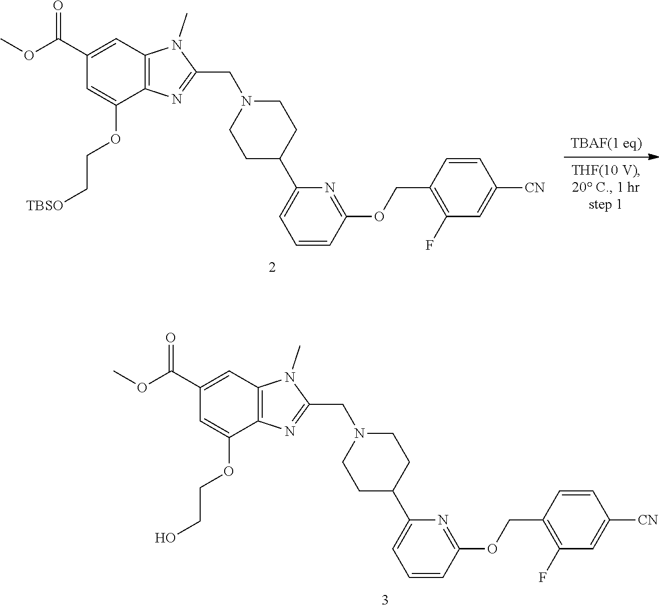 [SMILES: COC(=O)c1cc(OCCO[Si](C)(C)C(C)(C)C)c2nc(CN3CCC(c4cccc(OCc5ccc(C#N)cc5F)n4)CC3)n(C)c2c1 ; COC(=O)c1cc(OCCO)c2nc(CN3CCC(c4cccc(OCc5ccc(C#N)cc5F)n4)CC3)n(C)c2c1]

A mixture of 2 (80 mg, 116.30 μmol, 1 eq), TBAF (1 M, 116.30 μL, 1 eq) in THF (0.8 mL) was degassed and purged with N₂ for 3 times, and then the mixture was stirred at 20° C. for 12 hrs under N₂ atmosphere. LC-MS showed 2 was consumed completely and one main peak with desired mass was detected. The reaction mixture was diluted with H₂O 5 mL and extracted with EtOAc (5 mL*3). The combined organic layers were washed with brine 5 mL, dried over Na₂SO₄, filtered, and concentrated under reduced pressure to give a residue. 3 (60 mg, 104.60 μmol, 89.94% yield) was obtained as a brown solid.

LCMS: RT=0.911 min, MS cal.: 573.24, [M+H]⁺=574.3

### General Procedure for Preparation of Compound 291 (4):

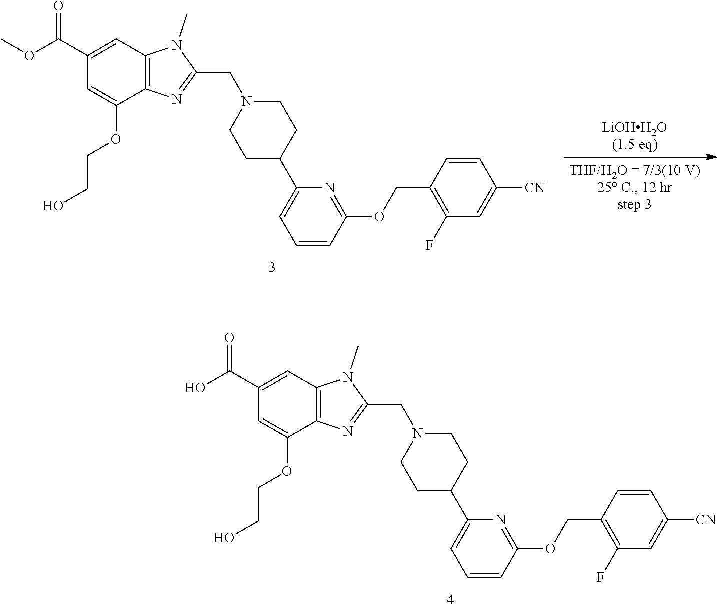 [SMILES: COC(=O)c1cc(OCCO)c2nc(CN3CCC(c4cccc(OCc5ccc(C#N)cc5F)n4)CC3)n(C)c2c1 ; Cn1c(CN2CCC(c3cccc(OCc4ccc(C#N)cc4F)n3)CC2)nc2c(OCCO)cc(C(=O)O)cc21]

A mixture of 3 (50 mg, 87.17 μmol, 1 eq), LiOH·H₂O (5.49 mg, 130.75 μmol, 1.5 eq) in THF (0.35 mL) and H₂O (0.15 mL) was degassed and purged with N₂ for 3 times, and then the mixture was stirred at 25° C. for 12 hrs under N₂ atmosphere. LC-MS showed 3 was consumed completely and one main peak with desired mass was detected. The reaction mixture was diluted with ACN 0.3 mL. The liquid was purified by prep-HPLC (column: Waters Xbridge Prep OBD C₁₈ 150*40 mm*10 um; mobile phase: [H₂O (10 mM NH₄HCO₃)-ACN]; gradient: 10%-60% B over 8.0 min). 2-((4-(6-((4-Cyano-2-fluorobenzyl)oxy)pyridin-2-yl)piperidin-1-yl)methyl)-4-(2-hydroxyethoxy)-1-methyl-1H-benzo[d]imidazole-6-carboxylic acid (29.66 mg, 53.00 μmol, 60.81% yield, 100.00% purity) was obtained as a white solid.

LCMS: RT=0.839 min, MS cal.: 559.22, [M+H]⁺=560.3

¹H NMR (400 MHz, METHANOL-d4) δ=7.79 (d, J=0.8 Hz, 1H), 7.67 (t, J=7.6 Hz, 1H), 7.62-7.51 (m, 3H), 7.45 (s, 1H), 6.83 (d, J=7.2 Hz, 1H), 6.67 (d, J=8.0 Hz, 1H), 5.51 (s, 2H), 4.27 (t, J=4.4 Hz, 2H), 4.01-3.96 (m, 5H), 3.87 (s, 2H), 3.01 (d, J=11.2 Hz, 2H), 2.70-2.54 (m, 1H), 2.35-2.21 (m, 2H), 1.87-1.78 (m, 4H)

### Example A226—Preparation of Compound 290. 2-((4-((6-((4-Cyano-2-fluorophenoxy)methyl)pyridin-2-yl)oxy)piperidin-1-yl)methyl)-4-ethoxy-1-methyl-1H-benzo[d]imidazole-6-carboxylic acid (Compound 290) (3)

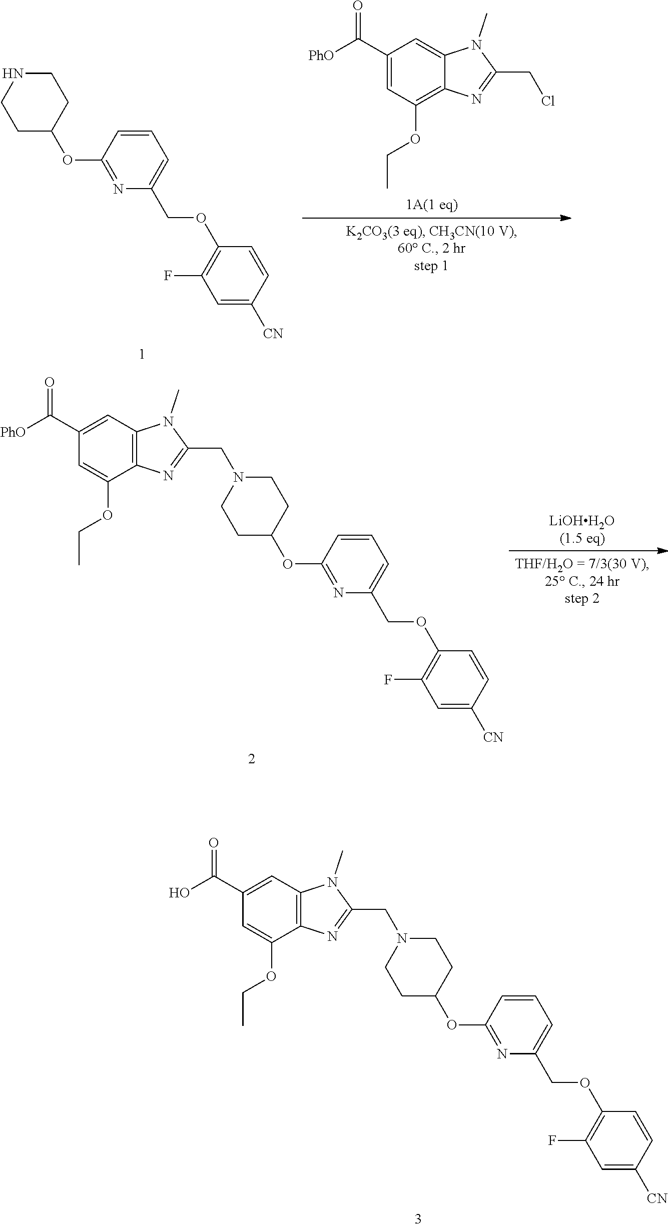 [SMILES: N#Cc1ccc(OCc2cccc(OC3CCNCC3)n2)c(F)c1 ; CCOc1cc(C(=O)Oc2ccccc2)cc2c1nc(CCl)n2C ; CCOc1cc(C(=O)Oc2ccccc2)cc2c1nc(CN1CCC(Oc3cccc(COc4ccc(C#N)cc4F)n3)CC1)n2C ; CCOc1cc(C(=O)O)cc2c1nc(CN1CCC(Oc3cccc(COc4ccc(C#N)cc4F)n3)CC1)n2C]

### General Procedure for Preparation of Intermediate 2:

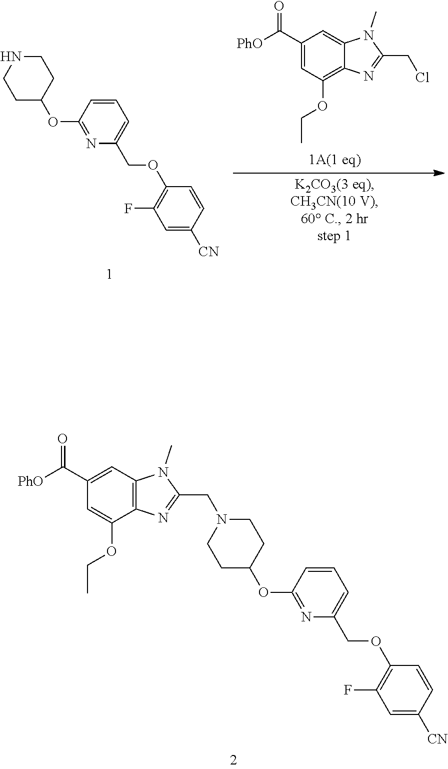 [SMILES: N#Cc1ccc(OCc2cccc(OC3CCNCC3)n2)c(F)c1 ; CCOc1cc(C(=O)Oc2ccccc2)cc2c1nc(CCl)n2C ; CCOc1cc(C(=O)Oc2ccccc2)cc2c1nc(CN1CCC(Oc3cccc(COc4ccc(C#N)cc4F)n3)CC1)n2C]

To a solution of compound 1 (100 mg, 305.48 μmol, 1 eq) and 1A (105.33 mg, 305.48 μmol, 1 eq) in CH₃CN (1 mL) was added K₂CO₃ (126.66 mg, 916.44 μmol, 3 eq) at 20° C. The mixture was stirred at 60° C. for 2 hr. LCMS (product: RT=0.472 min) showed the starting material was consumed completely. The reaction mixture was cooled down to 20° C. and was quenched by addition H₂O 5 mL at 20° C., and then extracted with EtOAc (8 mL*3). The combined organic layers were washed with brine (5 mL*2), dried over Na₂SO₄, filtered and concentrated under reduced pressure to give a residue. The residue was purified by column chromatography (SiO₂, PE/EtOAc=10/1 to 0/1). 2 (130 mg, 204.50 μmol, 66.94% yield) was obtained as a white solid. 1H NMR (400 MHz, CHLOROFORM-d) δ=7.93 (d, J=1.2 Hz, 1H), 7.60 (t, J=8.0 Hz, 1H), 7.53 (d, J=0.8 Hz, 1H), 7.49-7.43 (m, 2H), 7.43-7.36 (m, 2H), 7.30 (t, J=7.6 Hz, 1H), 7.27-7.23 (m, 2H), 7.10 (t, J=8.4 Hz, 1H), 7.02 (d, J=7.2 Hz, 1H), 6.66 (d, J=8.4 Hz, 1H), 5.19 (s, 2H), 5.07 (td, J=4.2, 8.0 Hz, 1H), 4.00 (s, 3H), 3.93 (s, 2H), 2.89-2.76 (m, 2H), 2.44 (t, J=9.2 Hz, 2H), 2.01 (d, J=10.4 Hz, 2H), 1.84-1.72 (m, 2H), 1.59 (t, J=7.2 Hz, 3H)

### General Procedure for Preparation of Compound 290 (3):

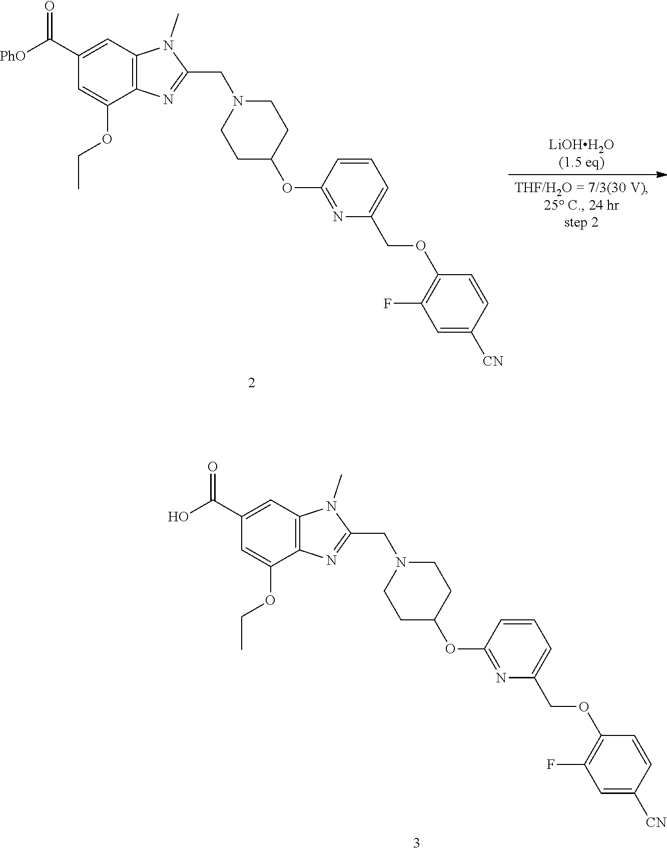 [SMILES: CCOc1cc(C(=O)Oc2ccccc2)cc2c1nc(CN1CCC(Oc3cccc(COc4ccc(C#N)cc4F)n3)CC1)n2C ; CCOc1cc(C(=O)O)cc2c1nc(CN1CCC(Oc3cccc(COc4ccc(C#N)cc4F)n3)CC1)n2C]

To a solution of 2 (100 mg, 157.31 μmol, 1 eq) in THF (2.1 mL) was added LiOH·H₂O (9.90 mg, 235.97 μmol, 1.5 eq) in H₂O (0.9 mL) at 25° C. The mixture was stirred at 25° C. for 24 hr. LCMS (product: RT=0.413 min; start material: RT=0.495 min) showed 2 was not consumed completely and one main peak with desired mass was detected. The reaction mixture was filtered. The filtrate was purified by prep-HPLC (column: Waters Xbridge Prep OBD C18 150*40 mm*10 um; mobile phase: [H₂O (10 mM NH₄HCO₃)—CH₃CN]; gradient: 15%-55% B over 8.0 min). 2-((4-((6-((4-Cyano-2-fluorophenoxy)methyl)pyridin-2-yl)oxy)piperidin-1-yl)methyl)-4-ethoxy-1-methyl-1H-benzo[d]imidazole-6-carboxylic acid (25.81 mg, 45.53 μmol, 28.94% yield, 98.72% purity) was obtained as a white solid.

LCMS: RT=2.534 min, MS cal.: 559.2, [M+H]⁺=560.2

HPLC: RT=9.960 min

¹H NMR (400 MHz, METHANOL-d4) δ=7.83 (d, J=0.8 Hz, 1H), 7.66 (t, J=8.0 Hz, 1H), 7.56 (dd, J=2.0, 10.8 Hz, 1H), 7.50 (d, J=8.8 Hz, 1H), 7.42 (s, 1H), 7.32 (t, J=8.4 Hz, 1H), 7.05 (d, J=7.2 Hz, 1H), 6.68 (d, J=8.4 Hz, 1H), 5.24 (s, 2H), 5.04 (qd, J=4.0, 7.8 Hz, 1H), 4.28 (q, J=7.2 Hz, 2H), 3.96 (s, 3H), 3.88 (s, 2H), 2.88-2.74 (m, 2H), 2.44 (t, J=9.2 Hz, 2H), 2.00 (br d, J=11.2 Hz, 2H), 1.85-1.67 (m, 2H), 1.52 (t, J=7.2 Hz, 3H)

### Example A227—Preparation of Compound 289. 2-((4-(6-((4-Cyano-2-fluorobenzyl)oxy)pyridin-2-yl)piperidin-1-yl)methyl)-3-methyl-7,8-dihydro-3H-[1,4]dioxino[2′,3′:3,4]benzo[1,2-d]imidazole-5-carboxylic acid (Compound 289) (11)

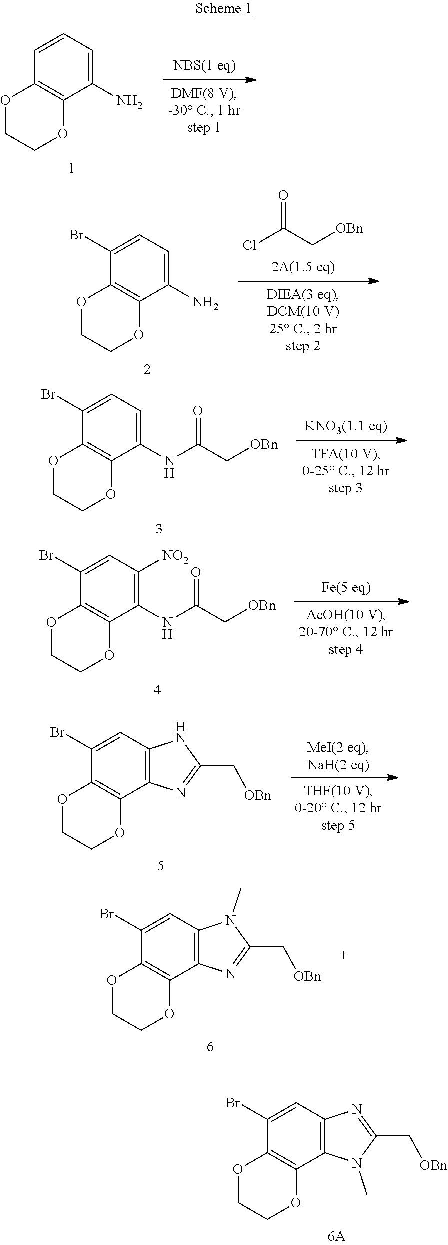

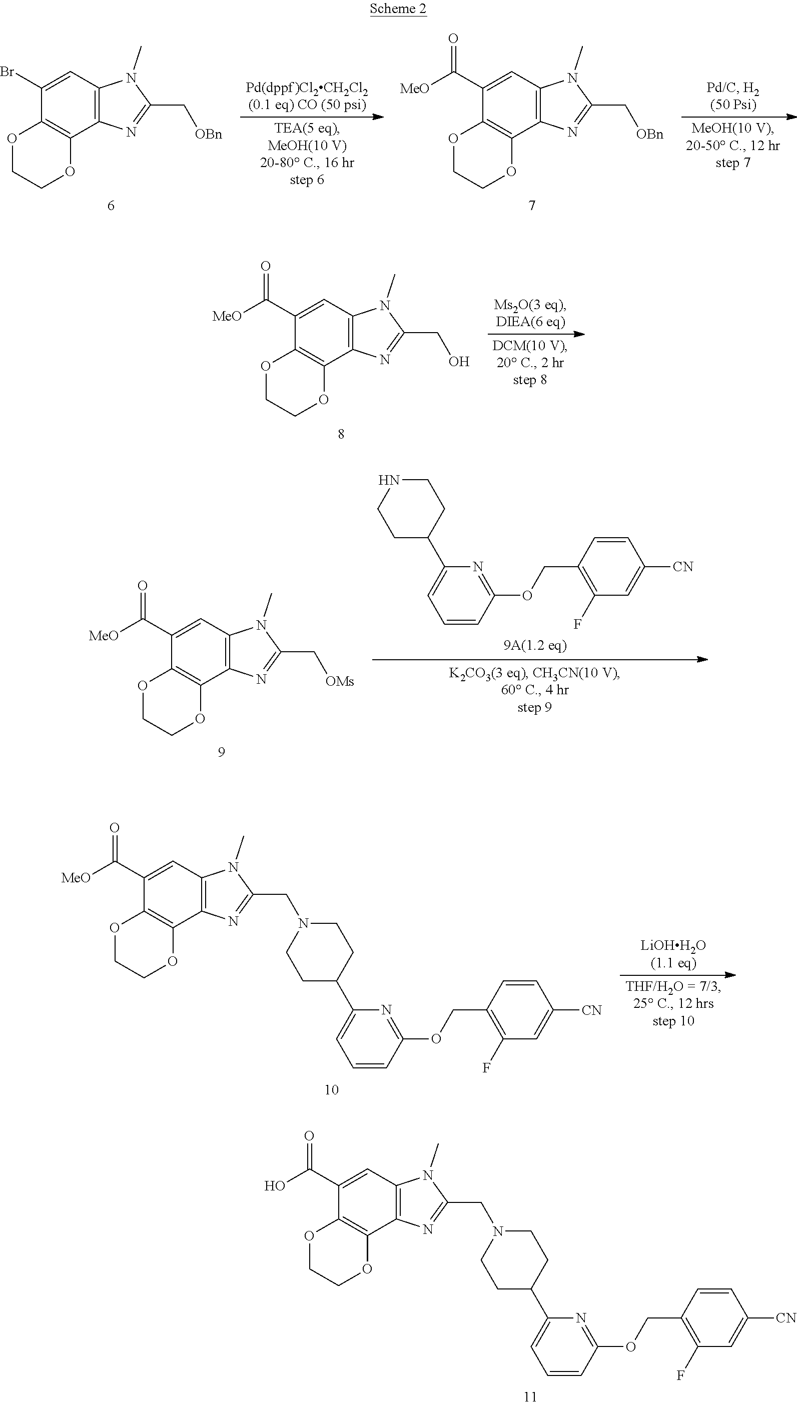

### General Procedure for Preparation of Scheme 1. Intermediate 2:

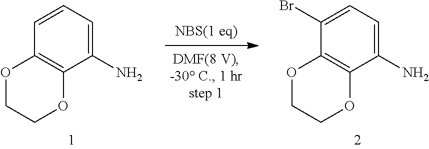 [SMILES: Nc1cccc2c1OCCO2 ; Nc1ccc(Br)c2c1OCCO2]

The reaction was set up in 2 batches.

A mixture of 1 (5 g, 33.08 mmol, 1 eq) in DMF (20 mL) was degassed and purged with N₂ for 3 times, then a solution of NBS (5.89 g, 33.08 mmol, 1 eq) in DMF (20 mL) was added dropwise to the mixture at −30° C. The mixture was stirred at −30° C. for 1 hr under N₂ atmosphere. TLC (PE/EtOAc=2/1, product R_{f}=0.47) showed 1 was consumed completely. Two batches were combined to work up. The mixture was poured into water 100 mL at 0° C. Solid was precipitate out. Then filtered and the filtered cake was co-evaporated with ACN 50 mL. The operation was repeated for twice. 2 (12 g, 52.16 mmol, 78.85% yield) was obtained as a pink solid.

LCMS: RT=0.999 min, MS cal.: 229.0/231.0, [M+H]⁺=230.0/232.0

¹H NMR (400 MHz, CHLOROFORM-d) δ=6.89 (d, J=8.4 Hz, 1H), 6.25 (d, J=8.4 Hz, 1H), 4.39-4.33 (m, 2H), 4.33-4.27 (m, 2H), 3.74 (br s, 2H)

### General Procedure for Preparation of Scheme 1, Intermediate 3:

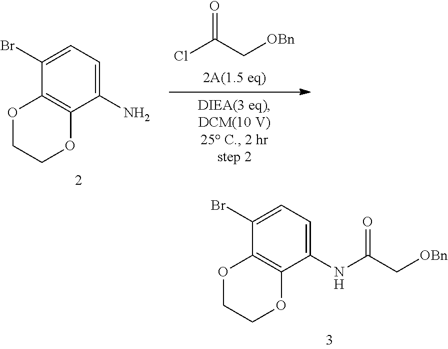 [SMILES: Nc1ccc(Br)c2c1OCCO2 ; O=C(Cl)COCc1ccccc1 ; O=C(COCc1ccccc1)Nc1ccc(Br)c2c1OCCO2]

The reaction was set up in 2 batches.

A mixture of 2 (6 g, 26.08 mmol, 1 eq), 2A (7.22 g, 39.12 mmol, 6.07 mL, 1.5 eq), DIEA (10.11 g, 78.24 mmol, 13.63 mL, 3 eq) in DCM (60 mL) was degassed and purged with N₂ for 3 times, and then the mixture was stirred at 25° C. for 2 hr under N₂ atmosphere. TLC (PE/EtOAc=2/1, product R_{f}=0.43) indicated 2 was consumed completely and one new spot formed. Two batches were combined to work up. The reaction mixture was quenched by addition H₂O 60 mL at 20° C. and extracted with DCM 40 mL*3. The combined organic layers were washed with brine 30 mL*5, dried over Na₂SO₄, filtered and concentrated under reduced pressure to give a residue. The residue was purified by column chromatography (SiO₂, Petroleum ether/Ethyl acetate=5/1 to 0/1). 3 (16 g, 42.30 mmol, 81.10% yield) was obtained as a white solid.

LCMS: RT=1.380 min, MS cal.: 377.0/379.0, [M+H]⁺=378.0/380.0

¹H NMR (400 MHz, CHLOROFORM-d) δ=8.83 (br s, 1H), 7.89 (d, J=9.2 Hz, 1H), 7.45-7.32 (m, 5H), 7.10 (d, J=9.2 Hz, 1H), 4.68 (s, 2H), 4.42-4.36 (m, 2H), 4.35-4.31 (m, 2H), 4.12 (s, 2H)

### General Procedure for Preparation of Scheme 1, Intermediate 4:

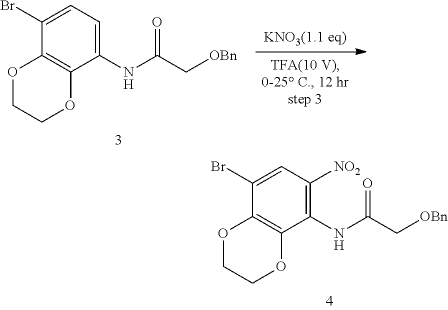 [SMILES: O=C(COCc1ccccc1)Nc1ccc(Br)c2c1OCCO2 ; O=C(COCc1ccccc1)Nc1c([N+](=O)[O-])cc(Br)c2c1OCCO2]

TFA (160 mL) was charged to a three-necked round bottom flask, then 3 (16 g, 42.30 mmol, 1 eq) was added to the mixture at 0° C. At 0° C., KNO₃ (4.70 g, 46.53 mmol, 1.1 eq) was added in portions to the reaction mixture at 0° C. After the addition, the mixture was stirred at 25° C. for 12 hr. TLC (PE/EtOAc=2/1, product R_{f}=0.35, byproduct R_{f}=0.21) showed the 3 was consumed completely. The reaction mixture was added dropwise to H₂O 150 mL at 0° C., then extracted with DCM 50 mL*4. The combined organic layers was added sat. Na₂CO₃ to adjust pH=8-9 at 0° C. and was poured into separatory funnel and separated. The organic layer was washed with brine 50 mL*2, dried over Na₂SO₄, filtered and the filtrate was concentrated to give a crude product. The residue was purified by column chromatography (SiO₂, Petroleum ether/Ethyl acetate=2/1 to 0/1). 4 (7.8 g, 18.43 mmol, 43.57% yield) was obtained as a white solid.

LCMS: RT=1.331 min, MS cal.: 422.0/424.0, [M+H]⁺=423.0/425.0

¹H NMR (400 MHz, CHLOROFORM-d) δ=8.95 (s, 1H), 7.92 (s, 1H), 7.46-7.33 (m, 5H), 4.71 (s, 2H), 4.48 (dd, J=3.2, 5.2 Hz, 2H), 4.36 (dd, J=3.2, 5.2 Hz, 2H), 4.16 (s, 2H)

### General Procedure for Preparation of Scheme 1, Intermediate 5:

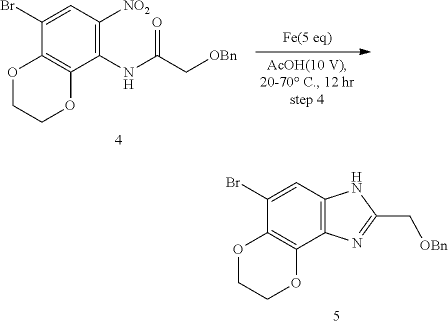 [SMILES: O=C(COCc1ccccc1)Nc1c([N+](=O)[O-])cc(Br)c2c1OCCO2 ; Brc1cc2[nH]c(COCc3ccccc3)nc2c2c1OCCO2]

The reaction was set up in 2 batches.

AcOH (45 mL) was charged to a 100 mL three-necked round bottom flask, then 4 (4.5 g, 10.63 mmol, 1 eq) was added to the mixture at 20° C. The mixture was heated to 50° C. At 50° C., Fe (2.97 g, 53.16 mmol, 5 eq) was added in portions to the reaction mixture at 50° C. After the addition, the mixture was stirred at 70° C. for 12 hr. LCMS (ET64759-704-P1A1, product: RT=1.147 min) showed the 4 was consumed completely, one main peak with desired mass was detected. The reaction mixture was cooled down to 30° C., then filtered through a pad of Celite, the filter cake was washed with EtOAc 30 mL*3. The combined organic layers were concentrated to give a residue. The residue was dissolved with EtOAc 60 mL, then washed with H₂O 20 mL, sat. Na₂CO₃ 20 mL*2 (until the aqueous layer's pH=9˜10), brine 20 mL*2, dried over Na₂SO₄, filtered, and concentrated under reduced pressure to give a residue. 5 (7.22 g, 19.24 mmol, 90.48% yield) was obtained as a brown solid.

LCMS: RT=1.331 min, MS cal.: 374.0/376.0, [M+H]⁺=375.0/377.0

¹H NMR (400 MHz, DMSO-d6) δ=7.40-7.26 (m, 6H), 4.65 (s, 2H), 4.57 (s, 2H), 4.37 (s, 4H)

### General Procedure for Preparation of Scheme 1, Intermediate 6 & Intermediate 6A:

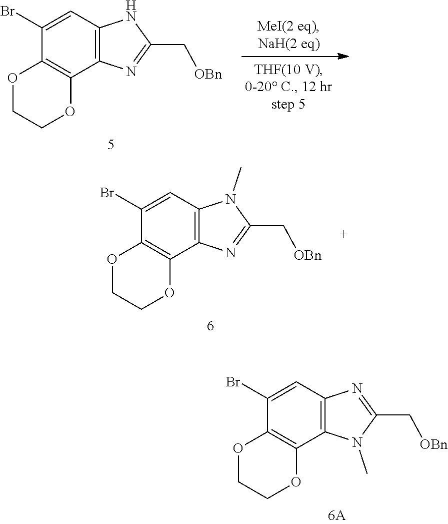 [SMILES: Brc1cc2[nH]c(COCc3ccccc3)nc2c2c1OCCO2 ; Cn1c(COCc2ccccc2)nc2c3c(c(Br)cc21)OCCO3 ; Cn1c(COCc2ccccc2)nc2cc(Br)c3c(c21)OCCO3]

To a solution of 5 (6 g, 15.99 mmol, 1 eq) in THF (60 mL) was added NaH (1.28 g, 31.98 mmol, 60% purity, 2 eq) in portions at 0° C. under N₂. The mixture was stirred at 0° C. for 0.5 hr at 0° C., MeI (4.54 g, 31.98 mmol, 1.99 mL, 2 eq) was added in portions to the reaction mixture. After the addition, the mixture was stirred at 20° C. for 11.5 hr. TLC (PE/EtOAc=1/1, 6 R_{f}=0.17, 6A R_{f}=0.34) showed compound 5 was consumed completely. The reaction mixture was quenched by addition H₂O 60 mL at 0° C. and extracted with EtOAc 50 mL*3. The combined organic layers were washed with brine 20 mL*2, dried over Na₂SO₄, filtered, and concentrated under reduced pressure to give a residue. The residue was purified by column chromatography (SiO₂, Petroleum ether/Ethyl acetate=5/1 to 0/1). 6 (3 g, 7.71 mmol, 48.20% yield) was obtained as a white solid. 6A (2 g, 5.14 mmol, 32.13% yield) was obtained as a white solid.

¹H NMR (400 MHz, CHLOROFORM-d) δ=7.37-7.28 (m, 5H), 7.13 (s, 1H), 4.84 (s, 2H), 4.55 (s, 2H), 4.48-4.40 (m, 4H), 3.76 (s, 3H)

¹H NMR (400 MHz, CHLOROFORM-d) δ=7.51 (s, 1H), 7.38-7.29 (m, 5H), 4.77 (s, 2H), 4.56 (s, 2H), 4.45-4.40 (m, 2H), 4.39-4.35 (m, 2H), 4.02 (s, 3H)

### General Procedure for Preparation of Scheme 2, Intermediate 7:

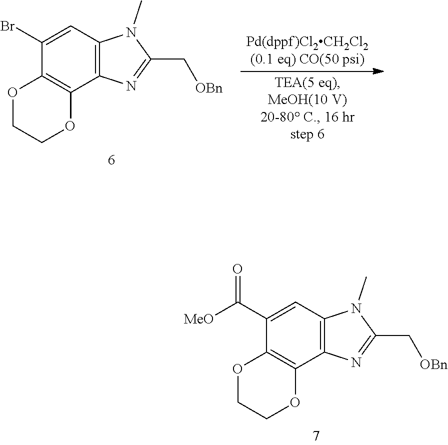 [SMILES: Cn1c(COCc2ccccc2)nc2c3c(c(Br)cc21)OCCO3 ; COC(=O)c1cc2c(nc(COCc3ccccc3)n2C)c2c1OCCO2]

MeOH (30 mL) was charged to a hydrogenating flask, 6 (3 g, 7.71 mmol, 1 eq) and TEA (3.90 g, 38.54 mmol, 5.36 mL, 5 eq) was added to the solution at 20° C. At 20° C., Pd(dppf)Cl₂·CH₂Cl₂ (629.41 mg, 770.73 μmol, 0.1 eq) was added to the reaction mixture under Ar atmosphere. The mixture was degassed under vacuum and purged with CO 3 times. The mixture was stirred under CO (50 psi) at 80° C. for 16 hours. LCMS (product: RT=1.121 min) showed the starting material was consumed completely, one main peak with desired mass was detected. The reaction mixture was cooled down 20° C. and filtered, the filter cake was washed EtOAc 10 mL*3. The filtrate was added H₂O 10 mL and FA (1 M) to adjust pH=6-7 and extracted with EtOAc 30 mL*3. The combined organic layers were washed with brine 20 mL*2, dried over Na₂SO₄, filtered, and concentrated under reduced pressure to give a residue. The residue was purified by column chromatography (SiO₂, Petroleum ether/Ethyl acetate=1/1 to 0/1). 7 (2.3 g, 6.24 mmol, 81.01% yield) was obtained as a red oil.

LCMS: RT=1.181 min, MS cal.: 368.1, [M+H]⁺=369.1

¹H NMR (400 MHz, CHLOROFORM-d) δ=7.49 (s, 1H), 7.37-7.28 (m, 5H), 4.87 (s, 2H), 4.57 (s, 2H), 4.51-4.47 (m, 2H), 4.45-4.41 (m, 2H), 3.94 (s, 3H), 3.82 (s, 3H)

### General Procedure for Preparation of Scheme 2, Intermediate 8:

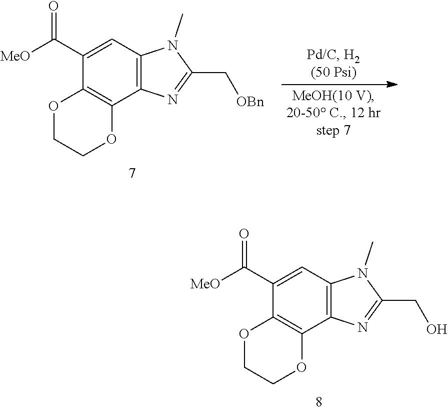 [SMILES: COC(=O)c1cc2c(nc(COCc3ccccc3)n2C)c2c1OCCO2 ; COC(=O)c1cc2c(nc(CO)n2C)c2c1OCCO2]

Solution A: Pd/C (300 mg) was added to hydrogenation bottle under Ar, then MeOH (1.5 mL) was added. 7 (300 mg, 814.37 μmol, 1 eq) in MeOH (1.5 mL) was added to solution A at 20° C. under Ar atmosphere. The mixture was degassed under vacuum and purged with H₂ 3 times. The mixture was stirred under H₂ (50 psi) at 50° C. for 12 hours. LCMS (ET64759-719-P1A1, product: RT=0.218 min) showed the 7 was consumed completely, one main peak with desired mass was detected. The suspension was filtered through a pad of Celite and the cake was washed with MeOH 20 mL×5. The combined filtrates were concentrated to give a product. 8 (220 mg, 790.63 μmol, 97.08% yield) was obtained as a colourless oil.

LCMS: RT=0.240 min, MS cal.: 278.1, [M+H]⁺=279.1

¹H NMR (400 MHz, CHLOROFORM-d) δ=7.47 (s, 1H), 4.90 (s, 2H), 4.48 (dd, J=2.8, 5.2 Hz, 2H), 4.45-4.39 (m, 2H), 3.93 (s, 3H), 3.80 (s, 3H), 3.49 (s, 1H)

### General Procedure for Preparation of Scheme 2, Intermediate 9:

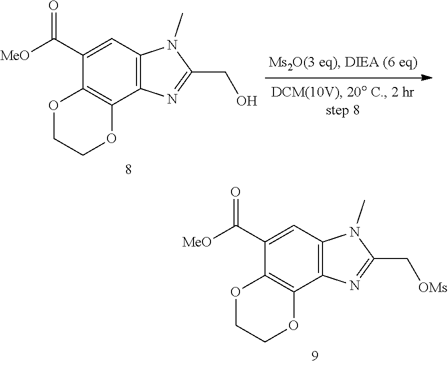 [SMILES: COC(=O)c1cc2c(nc(CO)n2C)c2c1OCCO2 ; COC(=O)c1cc2c(nc(COS(C)(=O)=O)n2C)c2c1OCCO2]

To a solution of 8 (200 mg, 718.75 μmol, 1 eq) and DIEA (557.36 mg, 4.31 mmol, 751.16 μL, 6 eq) in DCM (2 mL) was added Ms₂O (375.61 mg, 2.16 mmol, 3 eq) at 20° C. The mixture was stirred at 20° C. for 2 hr. LCMS (product: RT=0.353 min) showed the 8 was consumed completely. The reaction mixture was quenched by addition H₂O 5 mL at 20° C. and extracted with DCM 5 mL*3. The combined organic layers were washed with FA (1 M) to adjust pH=5-6, then the organic layer was washed with sat. Na₂CO₃ to adjust pH=8-9, and washed with brine 5 mL*2, dried over Na₂SO₄, filtered and concentrated under reduced pressure to give a residue. 9 (200 mg, crude) was obtained as a yellow oil.

### General Procedure for Preparation of Scheme 2, Intermediate 10:

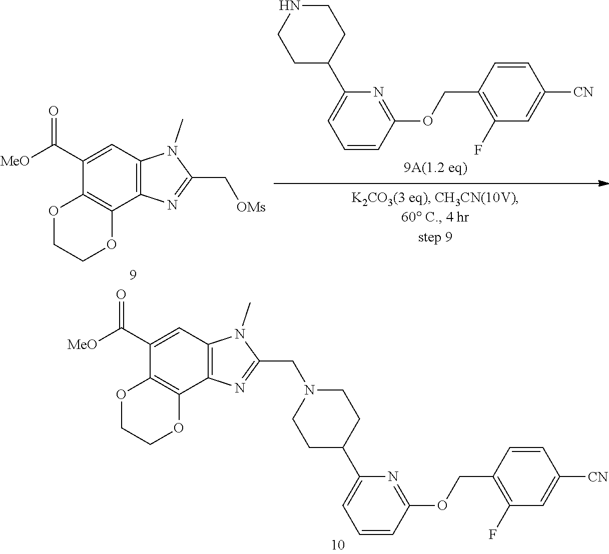 [SMILES: COC(=O)c1cc2c(nc(COS(C)(=O)=O)n2C)c2c1OCCO2 ; N#Cc1ccc(COc2cccc(C3CCNCC3)n2)c(F)c1 ; COC(=O)c1cc2c(nc(CN3CCC(c4cccc(OCc5ccc(C#N)cc5F)n4)CC3)n2C)c2c1OCCO2]

A mixture of 9 (100 mg, 224.50 μmol, 1 eq), 9A (83.88 mg, 269.40 μmol, 1.2 eq), K₂CO₃ (93.08 mg, 673.49 μmol, 3 eq), in CH₃CN (1 mL) was degassed and purged with N₂ for 3 times, and then the mixture was stirred at 60° C. for 4 hr under N₂ atmosphere. LC-MS (product, RT=1.385 min) showed 9 was consumed completely and one main peak with desired mass was detected. The reaction mixture was diluted with H₂O 10 mL and extracted with EtOAc 30 mL (10 mL*3). The combined organic layers were washed with brine 10 mL, dried over Na₂SO₄, filtered and concentrated under reduced pressure to give a residue. The residue was purified by column chromatography (SiO₂, Petroleum ether/Ethyl acetate=10/1 to 0/1). 10 (95 mg, 166.20 μmol, 74.03% yield) was obtained as a yellow solid.

LCMS: RT=1.385 min, MS cal.: 571.22, [M+H]⁺=572.1

¹H NMR (400 MHz, CHLOROFORM-d) δ=7.73-7.58 (m, 1H), 7.57-7.49 (m, 2H), 7.48-7.41 (m, 1H), 7.38 (br d, J=9.2 Hz, 1H), 6.82-6.74 (m, 1H), 6.71-6.60 (m, 1H), 5.55-5.46 (m, 2H), 4.54-4.37 (m, 5H), 4.17-4.04 (m, 1H), 4.00-3.79 (m, 6H), 3.05-2.89 (m, 1H), 2.69-2.51 (m, 1H), 2.33-2.21 (m, 1H), 1.93-1.65 (m, 4H), 0.95-0.79 (m, 2H)

### General Procedure for Preparation of Scheme 2, Compound 289 (11):

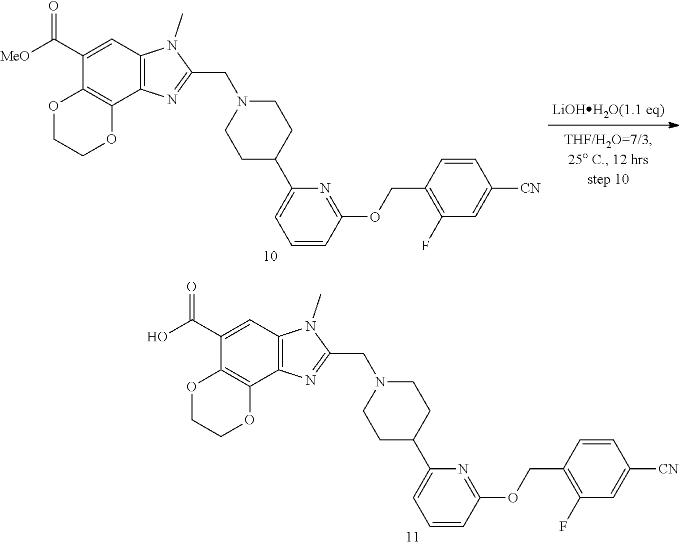 [SMILES: COC(=O)c1cc2c(nc(CN3CCC(c4cccc(OCc5ccc(C#N)cc5F)n4)CC3)n2C)c2c1OCCO2 ; Cn1c(CN2CCC(c3cccc(OCc4ccc(C#N)cc4F)n3)CC2)nc2c3c(c(C(=O)O)cc21)OCCO3]

A mixture of 10 (85 mg, 148.71 μmol, 1 eq), LiOH·H₂O (6.86 mg, 163.58 μmol, 1.1 eq) in THF (0.7 mL) and H₂O (0.3 mL) was degassed and purged with N₂ for 3 times, and then the mixture was stirred at 25° C. for 12 hr under N₂ atmosphere. LC-MS (ET83888-193, product, RT=1.009 min) showed 10 was consumed completely and one main peak with desired mass was detected. The reaction was purified directly. The residue was purified by prep-HPLC (column: Waters Xbridge Prep OBD C18 150*40 mm*10 um; mobile phase: [H₂O (10 mM NH₄HCO₃)-ACN]; gradient: 20%-60% B over 8.0 min). 2-((4-(6-((4-Cyano-2-fluorobenzyl)oxy)pyridin-2-yl)piperidin-1-yl)methyl)-3-methyl-7,8-dihydro-3H-[1,4]dioxino[2′,3′:3,4]benzo[1,2-d]imidazole-5-carboxylic acid (28.67 mg, 51.11 μmol, 34.37% yield, 99.40% purity) was obtained as a white solid.

LCMS: RT=2.154 min, MS cal.: 557.21, [M+1]⁺=558.2

¹H NMR (400 MHz, METHANOL-d4) δ=7.66 (t, J=7.2 Hz, 1H), 7.61-7.51 (m, 4H), 6.83 (d, J=7.2 Hz, 1H), 6.67 (d, J=8.4 Hz, 1H), 5.50 (s, 2H), 4.46-4.41 (m, 2H), 4.40-4.35 (m, 2H), 3.95-3.89 (m, 5H), 3.07 (br d, J=11.2 Hz, 2H), 2.65 (quin, J=7.6 Hz, 1H), 2.44-2.34 (m, 2H), 1.90-1.79 (m, 4H)

### Example A228—Preparation of Compound 288. (R)-2-((4-(3-(4-Cyano-2-fluorophenyl)-2,3-dihydrobenzo[b][1,4]dioxin-5-yl)piperidin-1-yl)methyl)-4-ethoxy-1-methyl-1H-benzo[d]imidazole-6-carboxylic acid (Compound 288) (3)

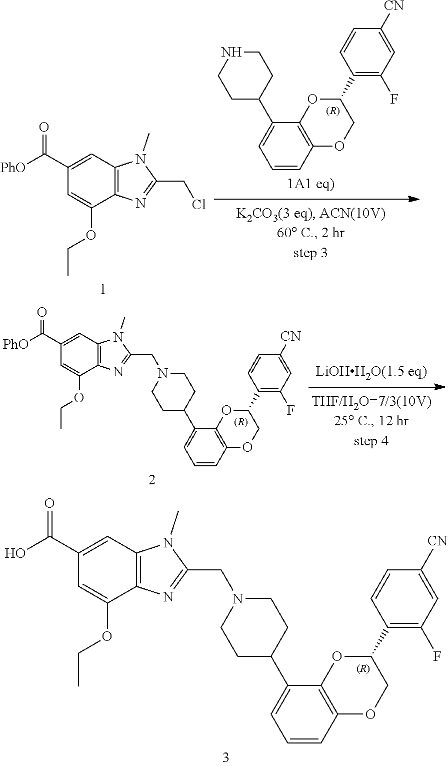

### General Procedure for Preparation of Intermediate 2:

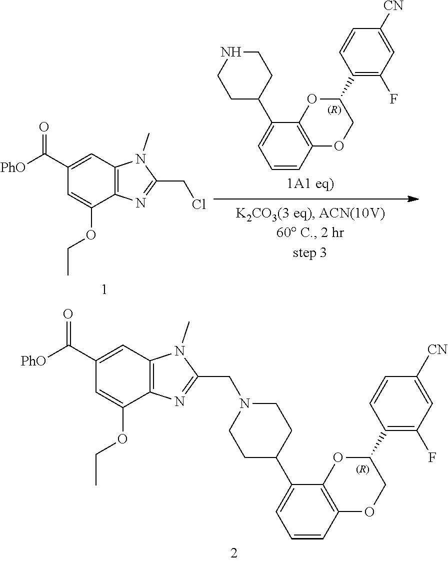

To a solution of 1 (100 mg, 221.04 μmol, 1 eq, TFA) in ACN (1 mL) was added K₂CO₃ (91.65 mg, 663.13 μmol, 3 eq) and 1A (76.21 mg, 221.04 μmol, 1 eq). The mixture was stirred at 60° C. for 2 hr. LCMS showed 1 was consumed completely and one main peak with desired mass was detected. The reaction mixture was diluted with H₂O 5 mL and extracted with EtOAc 15 mL (5 mL*3). The combined organic layers were washed with brine 10 mL, dried over Na₂SO₄, filtered and concentrated under reduced pressure to give a residue. The residue was purified by column chromatography (SiO₂, Petroleum ether/Ethyl acetate=1/0 to 1/1). 2 (80 mg, 121.23 μmol, 54.84% yield) was obtained as a white solid.

LCMS: RT=1.730 min, MS cal.: 646.3/647.3 [M+H]⁺=647.1/648.1

¹HNMR (400 MHz, DMSO-d6) δ=8.04-7.96 (m, 2H), 7.87-7.82 (m, 1H), 7.68 (t, J=7.6 Hz, 1H), 7.53-7.46 (m, 2H), 7.38 (s, 1H), 7.36-7.26 (m, 2H), 6.88-6.75 (m, 3H), 5.76 (s, 1H), 5.62-5.56 (m, 1H), 4.48 (dd, J=2.0, 11.4 Hz, 1H), 4.30 (q, J=7.0 Hz, 2H), 4.11 (dd, J=7.6, 11.5 Hz, 1H), 3.94 (s, 2H), 3.88-3.78 (m, 2H), 2.97-2.81 (m, 3H), 2.25-2.12 (m, 2H), 1.85-1.58 (m, 4H), 1.43 (t, J=6.8 Hz, 3H)

### General Procedure for Preparation of Compound 288 (3):

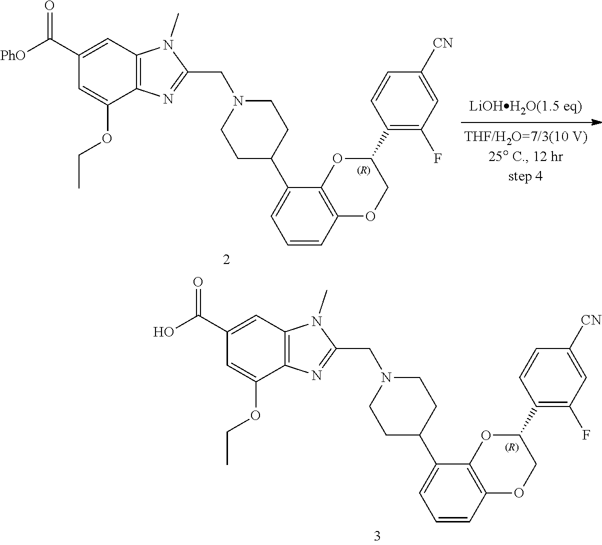

To a solution of 2 (70 mg, 108.24 μmol, 1 eq) in THF (0.7 mL) and H₂O (0.3 mL) was added LiOH·H₂O (6.81 mg, 162.36 μmol, 1.5 eq). The mixture was stirred at 25° C. for 12 hr. LCMS showed 27% of 2 remained. Several new peaks were shown on LCMS and 64% of desired MS of product was detected. The mixture was added THF 0.1 mL. The residue was purified by prep-HPLC (neutral condition: column: Waters X bridge Prep OBD C18 150*40 mm*10 um; mobile phase: [H₂O (10 mM NH₄HCO₃)-ACN]; gradient: 30%-80% B over 8.0 min). (R)-2-((4-(3-(4-Cyano-2-fluorophenyl)-2,3-dihydrobenzo[b][1,4]dioxin-5-yl)piperidin-1-yl)methyl)-4-ethoxy-1-methyl-1H-benzo[d]imidazole-6-carboxylic acid (25.88 mg, 44.90 μmol, 41.48% yield) was obtained as a white solid.

LCMS: RT=2.658 min, MS cal.: 570.2/571.2, [M+H]⁺=571.3/572.3

HPLC: RT=10.722 min

¹HNMR (400 MHz, DMSO-d6) δ=8.02-7.97 (m, 1H), 7.86-7.81 (m, 1H), 7.77-7.71 (m, 1H), 7.71-7.65 (m, 1H), 7.26-7.22 (m, 1H), 6.86-6.76 (m, 3H), 5.59 (dd, J=1.7, 7.2 Hz, 1H), 4.48 (dd, J=1.9, 11.1 Hz, 1H), 4.24 (q, J=6.8 Hz, 2H), 4.11 (dd, J=7.6, 11.6 Hz, 1H), 3.87 (s, 3H), 3.78 (d, J=3.2 Hz, 2H), 2.92 (br d, J=10.8 Hz, 3H), 2.21-2.11 (m, 2H), 1.83-1.76 (m, 1H), 1.83-1.56 (m, 3H), 1.41 (t, J=6.8 Hz, 3H)

### Example A229—Preparation of Compound 287. (S)-2-((4-(3-(4-Cyano-2-fluorophenyl)-2,3-dihydrobenzo[b][1,4]dioxin-5-yl)piperidin-1-yl)methyl)-4-ethoxy-1-methyl-1H-benzo[d]imidazole-6-carboxylic acid (Compound 287) (3)

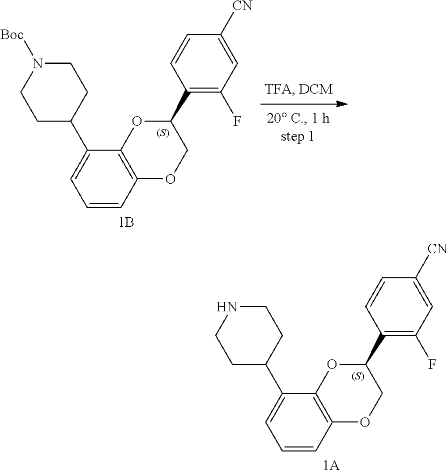

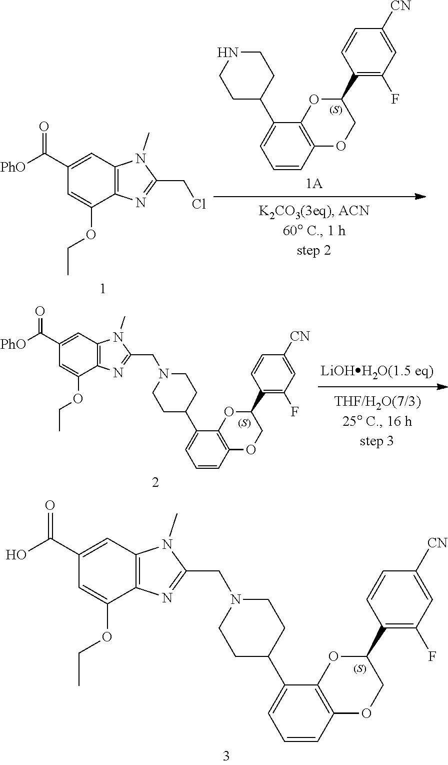

### General Procedure for Preparation of Intermediate 1A:

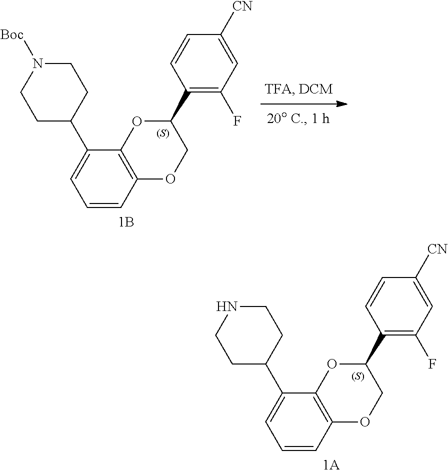

To a solution of 1B (50 mg, 114.03 μmol, 1 eq) in DCM (1.2 mL) was added TFA (0.3 mL). The mixture was stirred at 20° C. for 1 hr. LC-MS showed 1B was consumed completely and one main peak with desired mass was detected. The mixture was concentrated under reduced pressure. 1A (50 mg, 110.52 μmol, 96.93% yield, TFA) was obtained as yellow oil.

LCMS: RT=0.399 min, MS cal.: 338.1, [M+H]⁺=339.1

### General Procedure for Preparation of Intermediate 2:

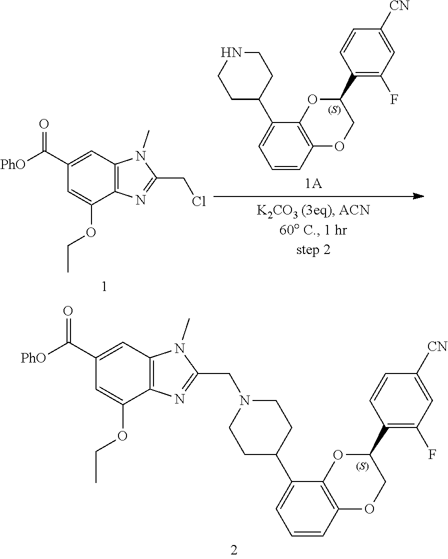

To a solution of 1 (50 mg, 145.02 μmol, 1 eq) in ACN (2 mL) was added K₂CO₃ (98.03 mg, 709.27 μmol, 3 eq) and 1A (49.20 mg, 108.76 μmol, 0.75 eq, TFA). The mixture was stirred at 60° C. for 2 hr. LCMS showed 1 was consumed completely and one main peak with desired mass was detected. The residue was poured into ice-water (5 mL) and stirred for 10 min. The aqueous phase was extracted with ethyl acetate (10 mL*3). The combined organic phase was washed with brine (10 mL), dried with anhydrous Na₂SO₄, filtered, and concentrated in vacuum. The residue was purified by prep-TLC (Ethyl acetate, Rf=0.18). 2 (45 mg, 69.58 μmol, 47.98% yield) was obtained as a white solid.

LCMS: RT=0.516 min, MS cal.: 646.3, [M+H]⁺=647.3

SFC: RT=1.431 min, ee %=100.0

¹H NMR (400 MHz, CHLOROFORM-d) δ=7.93 (s, 1H), 7.66-7.54 (m, 2H), 7.53 (s, 1H), 7.49-7.42 (m, 3H), 7.32-7.22 (m, 4H), 6.91-6.80 (m, 3H), 5.50 (br d, J=6.4 Hz, 1H), 4.46 (dd, J=1.6, 11.2 Hz, 1H), 4.37 (q, J=7.2 Hz, 2H), 4.02-3.96 (m, 4H), 3.91 (s, 2H), 3.04-2.90 (m, 3H), 2.30 (q, J=11.6 Hz, 2H), 1.90 (br d, J=12.0 Hz, 1H), 1.85-1.67 (m, 3H), 1.64-1.54 (m, 3H)

### General Procedure for Preparation of Compound 287 (3):

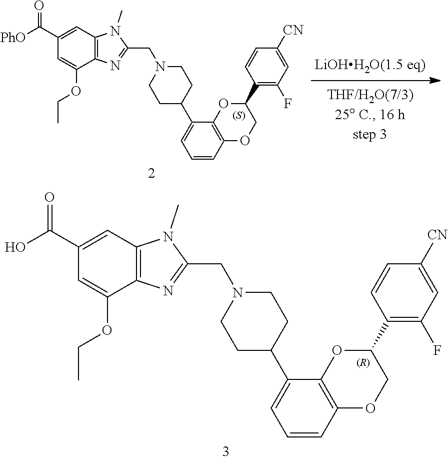

To a solution of 2 (45 mg, 69.58 μmol, 1 eq) in THF (1.4 mL) was added LiOH H₂O (8.82 mg, 210.30 μmol, 1.5 eq) in H₂O (0.6 mL). The mixture was stirred at 25° C. for 12 hr. LCMS showed 2 was consumed completely and one main peak with desired mass was detected. The mixture was purified directly. The crude was purified by prep-HPLC (column: Waters Xbridge Prep OBD C18 150*40 mm*10 um; mobile phase: [H₂O (10 mM NH₄HCO₃)-ACN]; gradient: 25%-65% B over 8.0 min). (S)-2-((4-(3-(4-Cyano-2-fluorophenyl)-2,3-dihydrobenzo[b][1,4]dioxin-5-yl)piperidin-1-yl)methyl)-4-ethoxy-1-methyl-1H-benzo[d]imidazole-6-carboxylic acid (29.49 mg, 51.68 μmol, 74.27% yield) was obtained as a white solid.

LCMS: RT=1.916 min, MS cal.: 570.2, [M+H]⁺=571.4

HPLC: RT=9.620 min, purity: 99.26%

SFC: RT=2.641 min, ee %=99.18

¹H NMR (400 MHz, DMSO-d6) δ=8.03-7.95 (m, 1H), 7.84 (dd, J=1.2, 8.0 Hz, 1H), 7.74 (s, 1H), 7.68 (t, J=7.4 Hz, 1H), 7.24 (s, 1H), 6.87-6.76 (m, 3H), 5.59 (dd, J=2.0, 7.1 Hz, 1H), 4.48 (dd, J=2.0, 11.6 Hz, 1H), 4.24 (q, J=7.2 Hz, 2H), 4.11 (dd, J=7.6, 11.6 Hz, 1H), 3.87 (s, 3H), 3.83-3.73 (m, 2H), 2.95-2.83 (m, 3H), 2.22-2.11 (m, 2H), 1.84-1.58 (m, 4H), 1.45-1.37 (m, 3H)

### Example A230—Preparation of Compound 286. 2-((4-(6-((4-Chloro-2-fluorophenyl)methoxy-d2)pyridin-2-yl)piperidin-1-yl)methyl)-4-(ethoxy-1,1-d2)-1-methyl-1H-benzo[d]imidazole-6-carboxylic acid (Compound 286) (3)

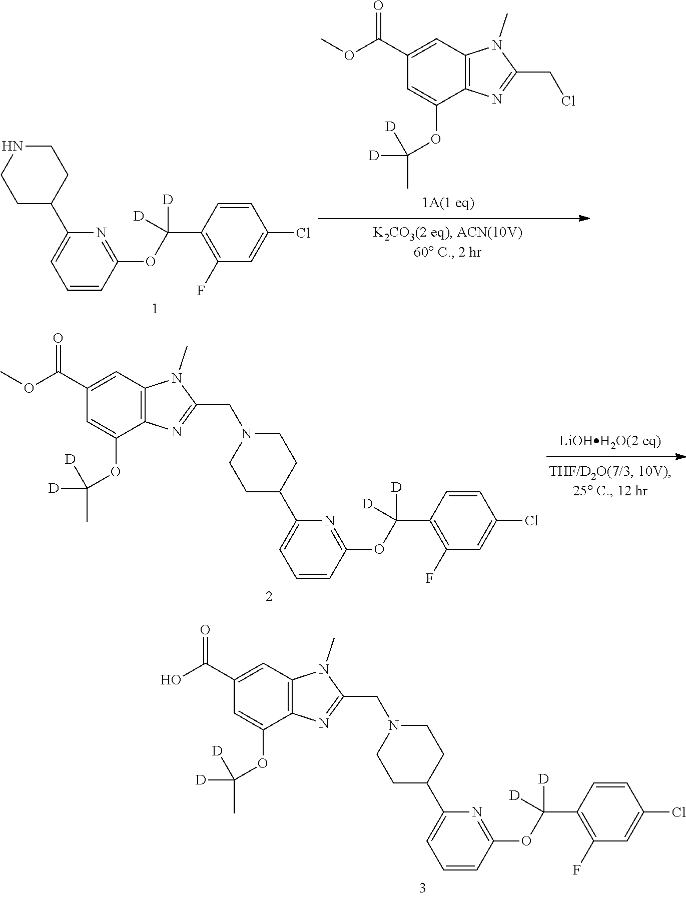 [SMILES: [2H]C([2H])(Oc1cccc(C2CCNCC2)n1)c1ccc(Cl)cc1F ; [2H]C([2H])(C)Oc1cc(C(=O)OC)cc2c1nc(CCl)n2C ; [2H]C([2H])(C)Oc1cc(C(=O)OC)cc2c1nc(CN1CCC(c3cccc(OC([2H])([2H])c4ccc(Cl)cc4F)n3)CC1)n2C ; [2H]C([2H])(C)Oc1cc(C(=O)O)cc2c1nc(CN1CCC(c3cccc(OC([2H])([2H])c4ccc(Cl)cc4F)n3)CC1)n2C]

### General Procedure for Preparation of Intermediate 2:

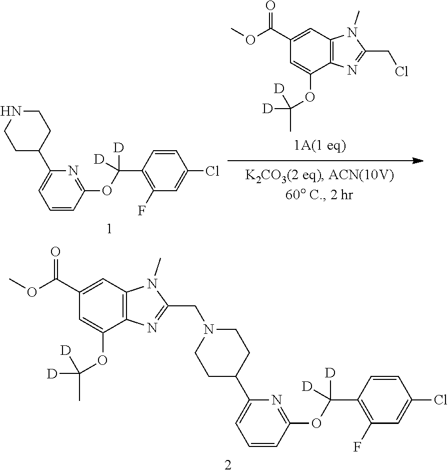 [SMILES: [2H]C([2H])(Oc1cccc(C2CCNCC2)n1)c1ccc(Cl)cc1F ; [2H]C([2H])(C)Oc1cc(C(=O)OC)cc2c1nc(CCl)n2C ; [2H]C([2H])(C)Oc1cc(C(=O)OC)cc2c1nc(CN1CCC(c3cccc(OC([2H])([2H])c4ccc(Cl)cc4F)n3)CC1)n2C]

To a solution of 1 (100 mg, 309.79 μmol, 1 eq) in ACN (1 mL) was added K₂CO₃ (85.63 mg, 619.58 μmol, 2 eq) and 1A (88.21 mg, 309.79 μmol, 1 eq). The mixture was stirred at 60° C. for 2 hr. LC-MS showed 1 was consumed completely and desired mass was detected. The mixture was diluted with H₂O (10 mL), and extracted by EtOAc (20 mL*2). Then organic phase was combined and washed by brine (10 mL), dried with Na₂SO₄, filtered. The filtrate was concentrated under reduced pressure at 40° C. to give residue. The residue was purified by column chromatography (SiO₂, Petroleum ether/Ethyl acetate=100/1 to 0/1). 2 (110 mg, 192.62 μmol, 62.18% yield) was obtained as a white solid.

Deuterated ratio: M+2:0.25%, M+0:5.16%, M+1:94.59%,

LCMS: RT=0.492 min, MS cal.: 571.0, [M+H]⁺=571.3

¹H NMR (400 MHz, CHLOROFORM-d) δ=7.76 (d, J=0.8 Hz, 1H), 7.50 (t, J=8.0 Hz, 1H), 7.44 (t, J=8.4 Hz, 1H), 7.39 (d, J=1.2 Hz, 1H), 7.14-7.07 (m, 2H), 6.73 (d, J=7.2 Hz, 1H), 6.60 (d, J=8.0 Hz, 1H), 3.98 (s, 3H), 3.96 (s, 3H), 3.90 (s, 2H), 2.96 (d, J=11.2 Hz, 2H), 2.60 (t, J=10.8 Hz, 1H), 2.28 (t, J=10.8 Hz, 2H), 1.92-1.73 (m, 4H), 1.55 (s, 3H)

### General Procedure for Preparation of Compound 286 (3):

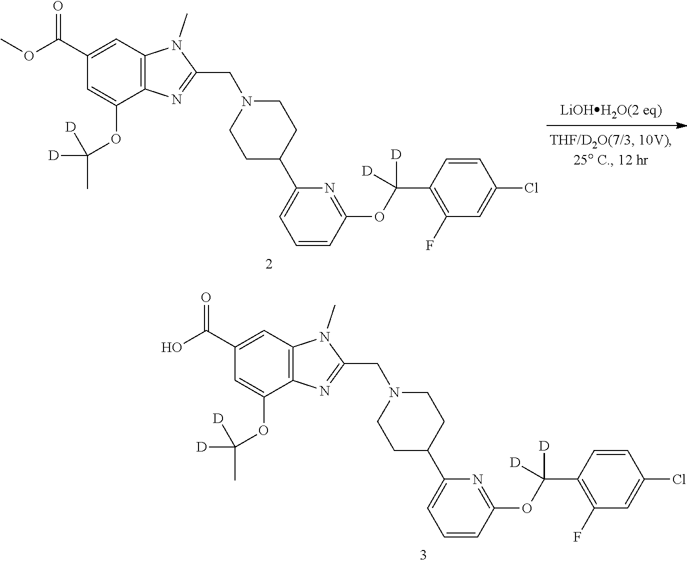 [SMILES: [2H]C([2H])(C)Oc1cc(C(=O)OC)cc2c1nc(CN1CCC(c3cccc(OC([2H])([2H])c4ccc(Cl)cc4F)n3)CC1)n2C ; [2H]C([2H])(C)Oc1cc(C(=O)O)cc2c1nc(CN1CCC(c3cccc(OC([2H])([2H])c4ccc(Cl)cc4F)n3)CC1)n2C]

To a solution of 2 (110 mg, 192.62 μmol, 1 eq) in THF (0.77 mL) and D₂O (0.33 mL) was added LiOH·H₂O (16.17 mg, 385.24 μmol, 2 eq). The mixture was stirred at 25° C. for 12 hr. LC-MS showed 2 was consumed completely and desired mass was detected. The mixture was concentrated under reduced pressure at 40° C. to give a residue. The crude product was purified by reversed-phase HPLC (column: Waters Xbridge Prep OBD C18 150*40 mm*10 um; mobile phase: [H₂O (10 mM NH₄HCO₃)-ACN]; gradient: 20%-70% B over 8.0 min). 2-((4-(6-((4-Chloro-2-fluorophenyl)methoxy-d2)pyridin-2-yl)piperidin-1-yl)methyl)-4-(ethoxy-1,1-d2)-1-methyl-1H-benzo[d]imidazole-6-carboxylic acid (29.03 mg, 52.11 μmol, 27.06% yield) was obtained as a white solid.

LCMS: RT=2.797 min, MS cal.: 557.0, [M+H]⁺=557.2

HPLC: RT=11.764 min, purity: 99.57%

¹H NMR (400 MHz, DMSO-d6) δ=7.75 (d, J=1.2 Hz, 1H), 7.62 (t, J=8.0 Hz, 1H), 7.56 (t, J=8.4 Hz, 1H), 7.45 (dd, J=2.4, 10.0 Hz, 1H), 7.28 (dd, J=2.0, 8.4 Hz, 1H), 7.25 (d, J=1.2 Hz, 1H), 6.86 (d, J=7.2 Hz, 1H), 6.66 (d, J=8.0 Hz, 1H), 3.90 (s, 3H), 3.80 (s, 2H), 2.92 (d, J=11.2 Hz, 2H), 2.63-2.53 (m, 1H), 2.25-2.12 (m, 2H), 1.84-1.62 (m, 4H), 1.40 (s, 3H)

### Example A231—Preparation of Compound 285. 2-((4-(6-((4-Cyano-2-fluorophenyl)methoxy-d2)pyridin-2-yl)piperidin-1-yl)methyl)-4-(ethoxy-1,1-d2)-1-methyl-1H-benzo[d]imidazole-6-carboxylic acid (Compound 285) (6)

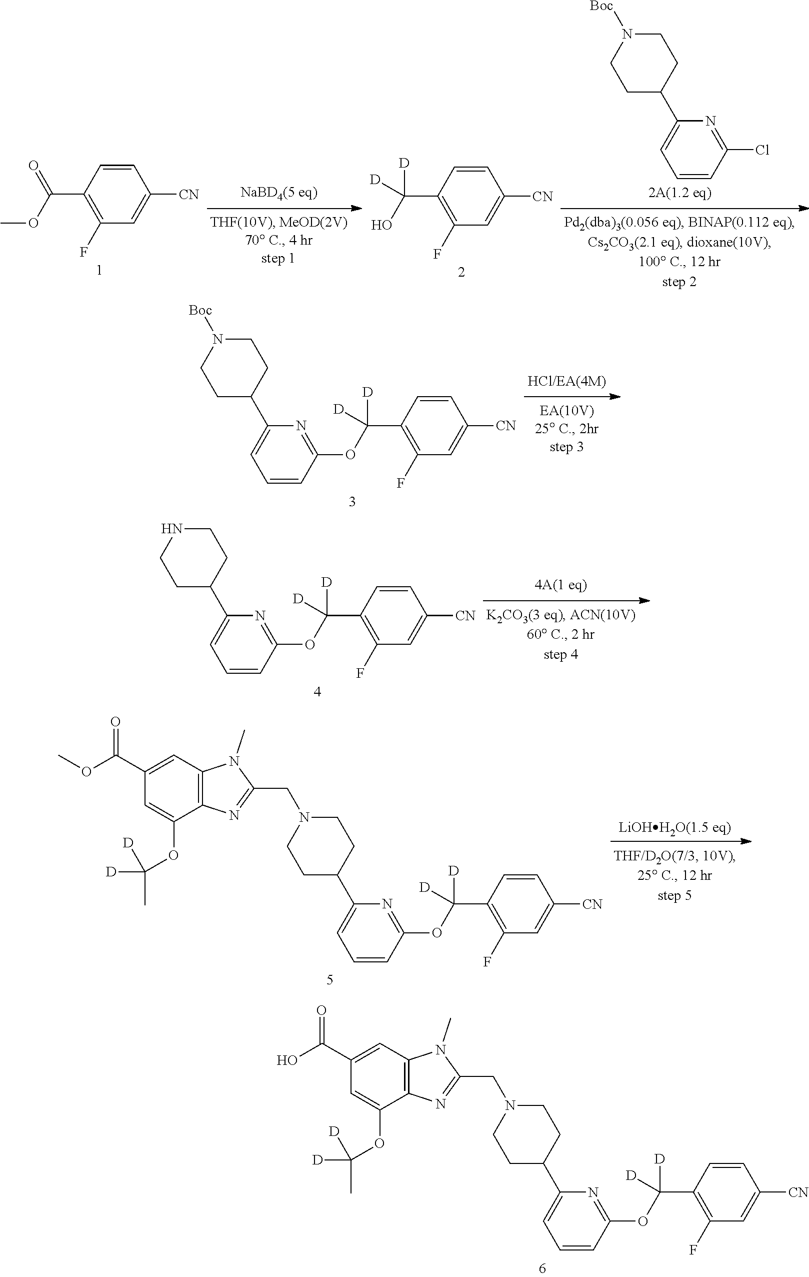

### General Procedure for Preparation of Intermediate 2:

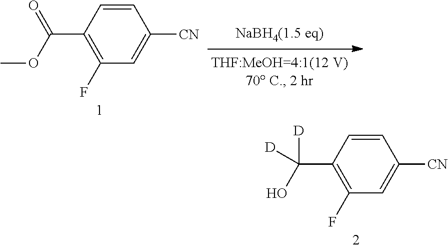

To a solution of 1 (4 g, 22.33 mmol, 1 eq) in THF (40 mL) and MeOH (10 mL) was added NaBH₄ (1.27 g, 33.49 mmol, 1.5 eq). The mixture was stirred at 70° C. for 2 hr. TLC (PE/EA=3/1, Rf=0.4) indicated 1 was consumed completely and one new spot formed. The reaction was clean according to TLC. The reaction mixture was quenched by addition D₂O 25 mL at 25° C., and then extracted with DCM 100 mL (20 mL*5). The combined organic layers were washed with brine 20 mL, dried over Na₂SO₄, filtered and concentrated under reduced pressure to give a residue. The residue was purified by column chromatography (SiO₂, PE/EA=1/0 to 3/2). 2 (2.48 g, 16.19 mmol, 72.52% yield) was obtained as a white solid.

¹HNMR (400 MHz, DMSO-d6) δ=7.78 (br d, J=10.0 Hz, 1H), 7.72-7.64 (m, 2H), 5.48 (s, 1H)

### General Procedure for Preparation of Intermediate 3:

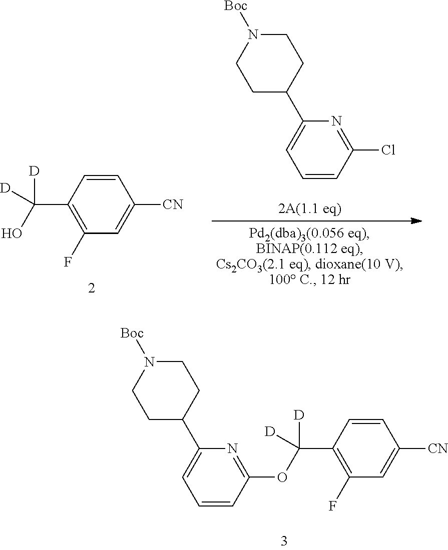

To a solution of 2 (2 g, 13.06 mmol, 1 eq) in dioxane (20 mL) was added Cs₂CO₃ (8.94 g, 27.42 mmol, 2.1 eq), BINAP (910.73 mg, 1.46 mmol, 0.112 eq), 2A (4.26 g, 14.37 mmol, 1.1 eq) and Pd₂(dba)₃ (669.67 mg, 731.31 μmol, 0.056 eq). The mixture was stirred at 100° C. for 12 hr. TLC (PE/EA=3/1, Rf=0.3) indicated 2 was consumed completely and many new spots formed. The reaction mixture was diluted with H₂O 20 mL and extracted with EtOAc 90 mL (30 mL*3). The combined organic layers were washed with brine 50 mL, dried over Na₂SO₄, filtered, and concentrated under reduced pressure to give a residue. The residue was purified by column chromatography (SiO₂, PE/EA=1/0 to 9/1). 3 (5.65 g, 12.43 mmol, 95.22% yield) was obtained as a yellow oil.

LCMS: RT=1.579 min, MS cal.: 413.2/414.2, [M−55]⁺=358.2/359.2

¹HNMR (400 MHz, DMSO-d6) δ=7.87-7.83 (m, 1H), 7.73-7.62 (m, 3H), 6.88 (d, J=7.2 Hz, 1H), 6.73 (d, J=8.4 Hz, 1H), 4.07-3.96 (m, 2H), 2.89-2.69 (m, 3H), 1.73 (d, J=11.2 Hz, 2H), 1.55-1.46 (m, 2H), 1.42 (s, 9H)

### General Procedure for Preparation of Intermediate 4:

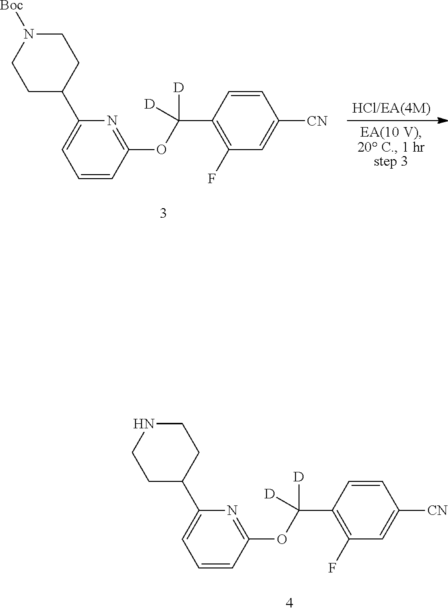 [SMILES: [2H]C([2H])(Oc1cccc(C2CCN(C(=O)OC(C)(C)C)CC2)n1)c1ccc(C#N)cc1F ; "EA(10 V),20° C., 1 hrstep 3" ; "HCl/EA(4M)" ; [2H]C([2H])(Oc1cccc(C2CCNCC2)n1)c1ccc(C#N)cc1F]

A mixture of 3 (5.3 g, 12.82 mmol, 1 eq) in EA (40 mL) and HCl/EA (10 mL) was degassed and purged with N₂ for 3 times, and then the mixture was stirred at 20° C. for 1 hr under N₂ atmosphere. LCMS showed 3 was consumed completely and one main peak with desired mass was detected. The reaction mixture was concentrated under reduced pressure to give a residue. 4 (4 g, 11.43 mmol, 89.20% yield, HCl) was obtained as a yellow solid.

LCMS: RT=0.364 min, MS cal.: 313.2, [M+H]⁺=314.1

### General Procedure for Preparation of Intermediate 5:

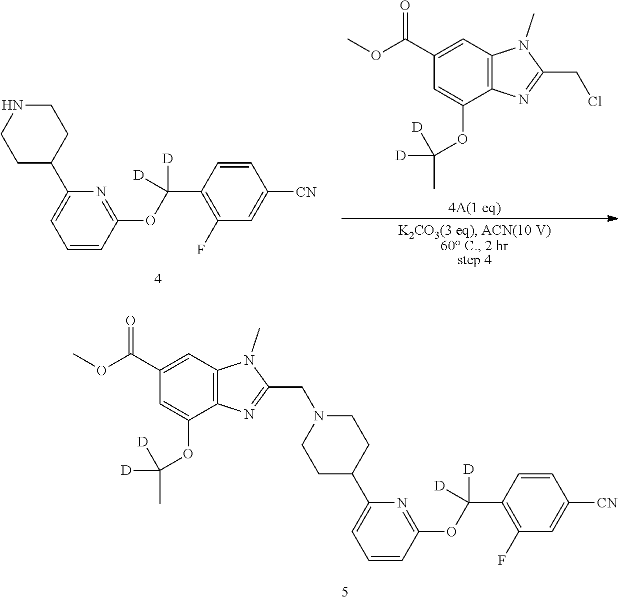

A mixture of 4 (100 mg, 319.12 μmol, 1 eq), 4A (90.86 mg, 319.12 μmol, 1 eq), K₂CO₃ (132.31 mg, 957.35 μmol, 3 eq) in ACN (1 mL) was degassed and purged with N₂ for 3 times, and then the mixture was stirred at 60° C. for 2 hr under N₂ atmosphere. LCMS showed 4 was consumed completely and one main peak with desired mass was detected. The reaction mixture was added with H₂O (10 mL) and extracted with EA (10 mL*3). The combined organic layers were washed with brine (10 mL*3), dried over Na₂SO₄, filtered and concentrated under reduced pressure to give a residue. The residue was purified by column chromatography (SiO₂, PE/EA=10/1 to 0/1). 5 (140 mg, 249.27 μmol, 78.11% yield) was obtained as a yellow solid.

LCMS: RT=1.556 min, MS cal.: 561.3, [M+H]⁺=562.2

### General Procedure for Preparation of Compound 285 (6):

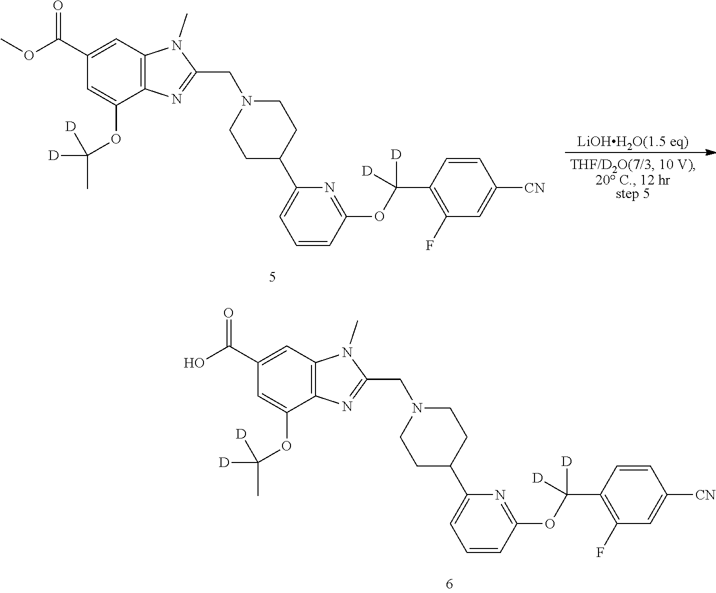 [SMILES: [2H]C([2H])(C)Oc1cc(C(=O)OC)cc2c1nc(CN1CCC(c3cccc(OC([2H])([2H])c4ccc(C#N)cc4F)n3)CC1)n2C ; "LiOH·H2O(1.5 eq)" ; "THF/D2O(7/3, 10 V),20° C., 12 hrstep 5" ; [2H]C([2H])(C)Oc1cc(C(=O)O)cc2c1nc(CN1CCC(c3cccc(OC([2H])([2H])c4ccc(C#N)cc4F)n3)CC1)n2C]

A mixture of 5 (50 mg, 89.03 μmol, 1 eq), LiOH·H₂O (5.60 mg, 133.54 μmol, 1.5 eq) in THF (0.7 mL) and D₂O (0.3 mL) was degassed and purged with N₂ for 3 times, and then the mixture was stirred at 20° C. for 12 hr under N₂ atmosphere. LCMS showed 5 was consumed completely and one main peak with desired mass was detected. The reaction mixture was added with H₂O (5 mL) and extracted with EtOAc (5 mL*3). The combined organic layers were washed with brine (5 mL*3), dried over Na₂SO₄, filtered, and concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 20%-60% B over 8.0 min). 2-((4-(6-((4-Cyano-2-fluorophenyl)methoxy-d2)pyridin-2-yl)piperidin-1-yl)methyl)-4-(ethoxy-1,1-d2)-1-methyl-1H-benzo[d]imidazole-6-carboxylic acid (25.2 mg, 46.02 μmol, 51.69% yield) was obtained as a white solid.

LCMS: RT=2.619 min, MS cal.: 547.3, [M+H]⁺=548.3

HPLC: RT=10.276 min, purity: 98.51%

¹H NMR (400 MHz, DMSO-d6) δ=7.88 (d, J=9.6 Hz, 1H), 7.77 (d, J=1.2 Hz, 1H), 7.69 (d, J=3.6 Hz, 2H), 7.66-7.60 (m, 1H), 7.24 (d, J=1.2 Hz, 1H), 6.87 (d, J=7.2 Hz, 1H), 6.70 (d, J=8.0 Hz, 1H), 3.90 (s, 3H), 3.80 (s, 2H), 2.90 (d, J=10.8 Hz, 2H), 2.62-2.53 (m, 1H), 2.18 (br t, J=10.6 Hz, 2H), 1.80-1.59 (m, 4H), 1.40 (s, 3H)

### Example A232—Preparation of Compound 284. 2-((4-(3-((4-Chloro-2-fluorobenzyl)oxy)phenyl)piperidin-1-yl)methyl)-4-(ethoxy-1,1-d2)-1-methyl-1H-benzo[d]imidazole-6-carboxylic acid (Compound 284) (3)

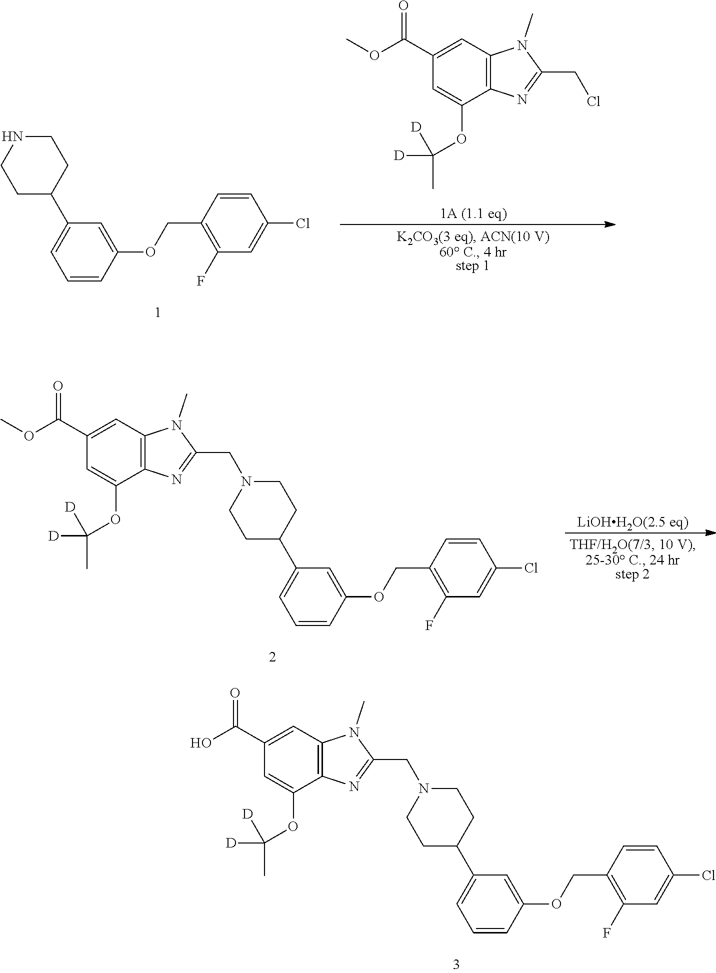

### General Procedure for Preparation of Intermediate 2:

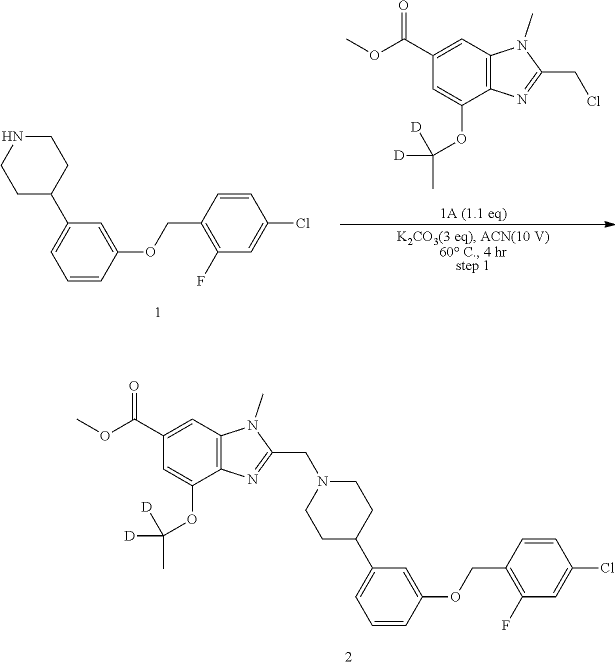 [SMILES: Fc1cc(Cl)ccc1COc1cccc(C2CCNCC2)c1 ; [2H]C([2H])(C)Oc1cc(C(=O)OC)cc2c1nc(CCl)n2C ; "1A (1.1 eq)" ; "K2CO3(3 eq), ACN(10 V)60° C., 4 hrstep 1" ; [2H]C([2H])(C)Oc1cc(C(=O)OC)cc2c1nc(CN1CCC(c3cccc(OCc4ccc(Cl)cc4F)c3)CC1)n2C]

To a solution of 1 (100 mg, 312.70 μmol, 1 eq) in CH₃CN (1 mL) was added K₂CO₃ (129.65 mg, 938.09 μmol, 3 eq) and 1 (89.04 mg, 312.70 μmol, 1 eq). The mixture was stirred at 60° C. for 2 hr. After monitoring, the mixture was added 1A (8.90 mg, 31.27 μmol, 0.1 eq). The mixture was stirred at 60° C. for 2 hr. LCMS showed 1 was consumed completely and one main peak with desired mass was detected. The crude reaction mixture on notebook page ET78169-383 was combined to ET78169-384 for workup. The reaction mixture was diluted with H₂O 5 mL and extracted with EtOAc 30 mL (10 mL*3). The combined organic layers were washed with brine 20 mL, dried over Na₂SO₄, filtered and concentrated under reduced pressure to give a residue. The residue was purified by column chromatography (SiO₂, Petroleum ether/Ethyl acetate=1/0 to 1/1). 2 (130 mg, 219.69 μmol, 70.26% total yield) was obtained as a white solid.

LCMS: RT=1.710 min, MS cal.: 567.2/568.2, [M+H]⁺=568.2/569.2

¹HNMR (400 MHz, DMSO-d6) δ=7.80 (s, 1H), 7.58 (t, J=8.0 Hz, 1H), 7.52-7.46 (m, 1H), 7.33 (d, J=8.4 Hz, 1H), 7.24 (s, 1H), 7.22-7.17 (m, 1H), 6.89 (s, 1H), 6.86-6.80 (m, 2H), 5.09 (s, 2H), 3.91 (s, 3H), 3.87 (s, 3H), 3.80 (s, 2H), 2.92 (br d, J=11.2 Hz, 2H), 2.46 (br s, 2H), 2.18 (br t, J=10.8 Hz, 2H), 1.77-1.55 (m, 4H), 1.40 (s, 3H)

### General Procedure for Preparation of Compound 284 (3):

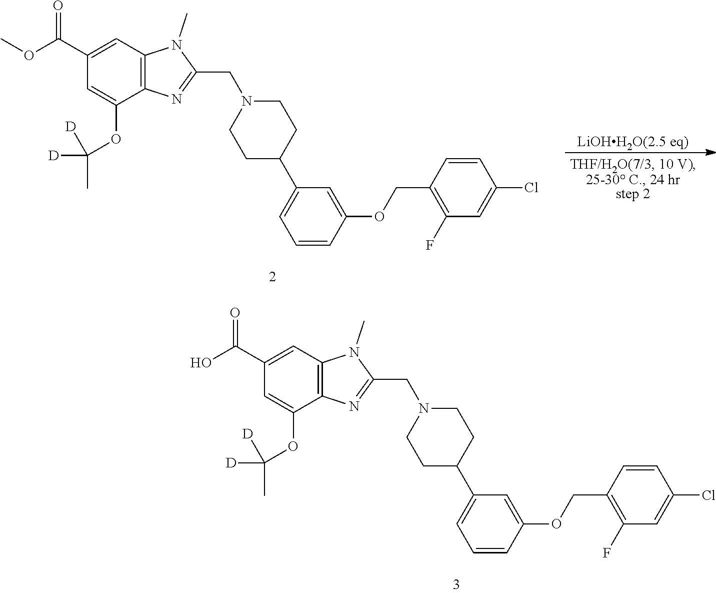 [SMILES: [2H]C([2H])(C)Oc1cc(C(=O)OC)cc2c1nc(CN1CCC(c3cccc(OCc4ccc(Cl)cc4F)c3)CC1)n2C ; "LiOH·H2O(2.5 eq)" ; "THF/H2O(7/3, 10 V),25-30° C., 24 hrstep 2" ; [2H]C([2H])(C)Oc1cc(C(=O)O)cc2c1nc(CN1CCC(c3cccc(OCc4ccc(Cl)cc4F)c3)CC1)n2C]

To a solution of 2 (110 mg, 193.64 μmol, 1 eq) in THF (0.7 mL) and H₂O (0.3 mL) was added LiOH·H₂O (12.19 mg, 290.46 μmol, 1.5 eq). The mixture was stirred at 25° C. for 12 hr. After monitoring, the mixture was added LiOH·H₂O (8.13 mg, 193.64 μmol, 1 eq). The mixture was stirred at 30° C. for 12 hr. LCMS showed compound 2 was consumed completely and one main peak with desired mass was detected. The mixture was added THF 0.1 mL. The residue was purified by prep-HPLC (neutral condition: column: Waters Xbridge Prep OBD C18 150*40 mm*10 um; mobile phase: [H₂O (10 mM NH₄HCO₃)-ACN]; gradient: 20%-70% B over 8.0 min). 2-((4-(3-((4-Chloro-2-fluorobenzyl)oxy)phenyl)piperidin-1-yl)methyl)-4-(ethoxy-1,1-d2)-1-methyl-1H-benzo[d]imidazole-6-carboxylic acid (29.51 mg, 53.26 μmol, 27.51% yield) was obtained as a white solid.

LCMS: RT=2.837 min, MS cal.: 553.2/554.2, [M+H]⁺=554.2/555.2

HPLC: RT=12.200 min

¹HNMR (400 MHz, DMSO-d6) δ=7.75-7.71 (m, 1H), 7.58 (t, J=8.4 Hz, 1H), 7.49 (dd, J=2.0, 10.0 Hz, 1H), 7.35-7.30 (m, 1H), 7.25 (s, 1H), 7.23-7.17 (m, 1H), 6.89 (s, 1H), 6.86-6.80 (m, 2H), 5.12-5.06 (m, 2H), 3.88 (s, 3H), 3.79 (s, 2H), 2.93 (br d, J=11.2 Hz, 2H), 2.47 (br s, 1H), 2.23-2.12 (m, 2H), 1.77-1.56 (m, 4H), 1.39 (s, 3H)

### Example A233—Preparation of Compound 283. 2-((4-(6-((4-Cyano-2,3-dihydrobenzofuran-7-yl)methoxy)pyridin-2-yl)piperidin-1-yl)methyl)-4-methoxy-1-methyl-1H-benzo[d]imidazole-6-carboxylic acid (Compound 283) (5)

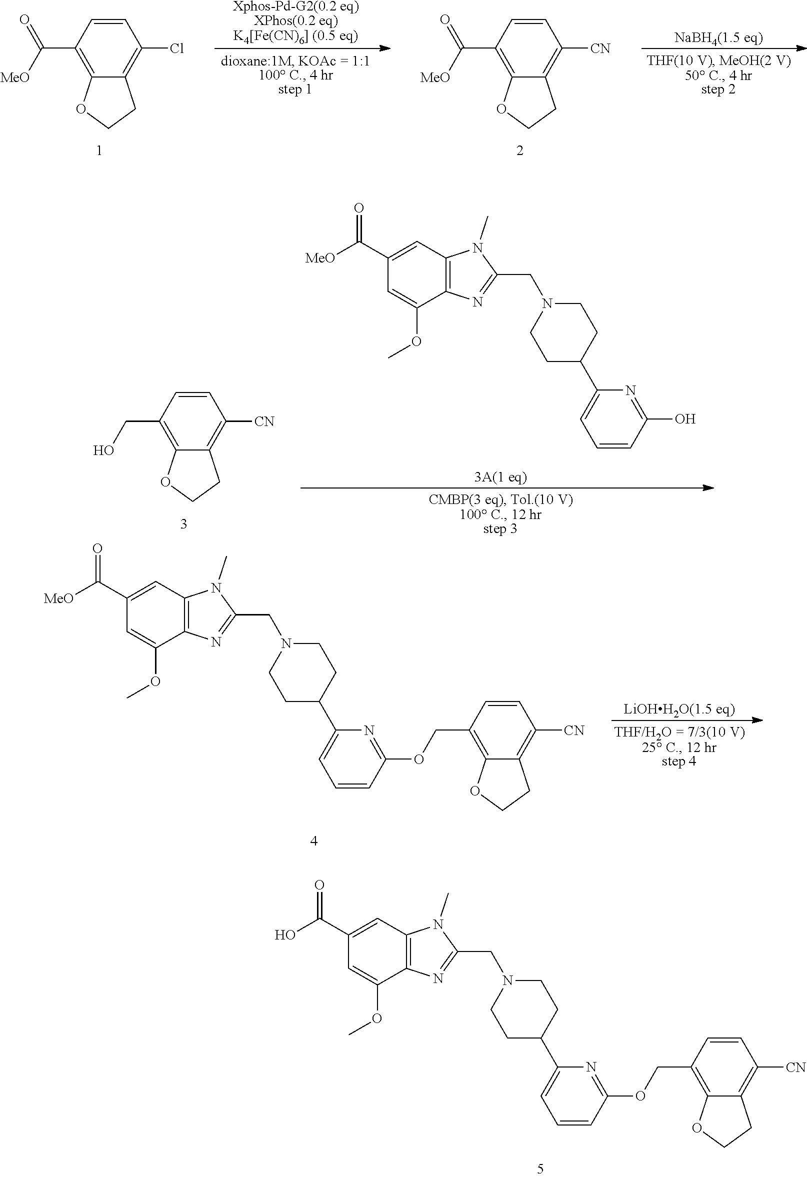

### General Procedure for Preparation of Intermediate 2:

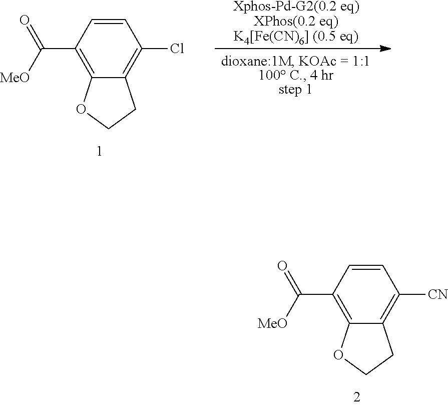

A mixture of 1 (300 mg, 1.41 mmol, 1 eq), Xphos-Pd-G2 (222.02 mg, 282.18 μmol, 0.2 eq), XPhos (134.52 mg, 282.18 μmol, 0.2 eq), K₄[Fe(CN)₆] (259.85 mg, 705.45 μmol, 0.5 eq) in dioxane (3 mL) and KOAc (3 mL, 0.05M) was degassed and purged with N₂ for 3 times, and then the mixture was stirred at 100° C. for 4 hrs under N₂ atmosphere. TLC indicated 1 was consumed completely and one new spot formed Petroleum ether:Ethyl acetate=2:1, product R_{f}=0.42. The residue mixture was diluted with H₂O 20 mL and extracted with EtOAc (20 mL*3). The combined organic layers were washed with brine 20 mL, dried over Na₂SO₄, filtered and concentrated under reduced pressure to give a residue. The residue was purified by column chromatography (SiO₂, Petroleum ether/Ethyl acetate=100/1 to 5/1, TLC-Petroleum ether/Ethyl acetate=5/1, Product R_{f}=0.42). 2 (240 mg, 1.18 mmol, 83.71% yield) was obtained as a white solid.

LCMS: RT=0.994 min, MS cal.: 203.06, [M+H]⁺=203.9

¹H NMR (400 MHz, CHLOROFORM-d) δ=7.82 (d, J=8.0 Hz, 1H), 7.14 (d, J=8.0 Hz, 1H), 4.84 (t, J=8.8 Hz, 2H), 3.94 (s, 3H), 3.45 (t, J=8.8 Hz, 2H)

### General Procedure for Preparation of Intermediate 3:

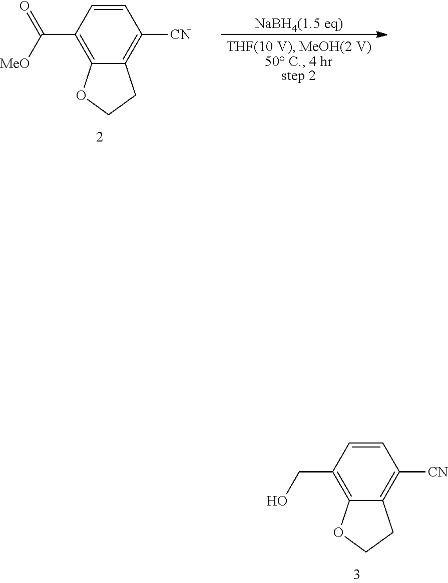 [SMILES: COC(=O)c1ccc(C#N)c2c1OCC2 ; "NaBH4(1.5 eq)" ; "THF(10 V), MeOH(2 V)50° C., 4 hrstep 2" ; N#Cc1ccc(CO)c2c1CCO2]

A mixture of 2 (200 mg, 984.28 μmol, 1 eq) in THF (2 mL) and MeOH (0.4 mL) was added NaBH₄ (55.86 mg, 1.48 mmol, 1.5 eq), degassed and purged with N₂ for 3 times, and then the mixture was stirred at 50° C. for 3 hr under N₂ atmosphere. NaBH₄ (55.86 mg, 1.48 mmol, 1.5 eq) was added to the reaction mixture at 50° C. After addition, the mixture was stirred at 50° C. for 2 hr under N₂ atmosphere. LC-MS showed 2 was consumed completely and one main peak with desired mass was detected. The reaction mixture was added to H₂O 10 mL at 0° C. and extracted with DCM (30 mL*3). The combined organic layers were washed with brine 30 mL, dried over Na₂SO₄, filtered, and concentrated under reduced pressure to give a residue. The residue was purified by prep-TLC (SiO₂, Petroleum ether:Ethyl acetate=2:1). 3 (140 mg, 799.16 μmol, 81.19% yield) was obtained as a white solid.

LCMS: RT=0.824 min, MS cal.: 175.06, [M−H]⁻=174.0

¹H NMR (400 MHz, CHLOROFORM-d) δ=7.37-7.32 (m, 1H), 7.29-7.24 (m, 1H), 5.39-5.29 (m, 1H), 4.64 (t, J=8.8 Hz, 2H), 4.47 (d, J=4.8 Hz, 2H), 3.40-3.34 (m, 2H)

### General Procedure for Preparation of Intermediate 4:

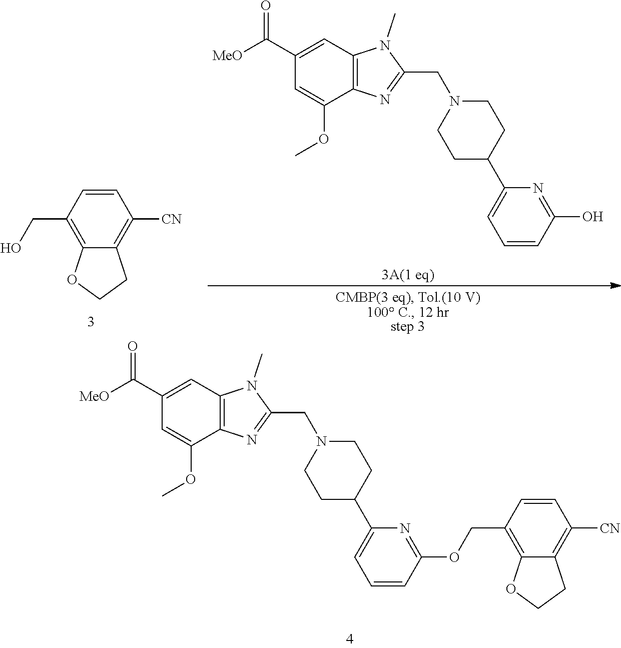 [SMILES: N#Cc1ccc(CO)c2c1CCO2 ; COC(=O)c1cc(OC)c2nc(CN3CCC(c4cccc(O)n4)CC3)n(C)c2c1 ; "3A(1 eq)" ; "CMBP(3 eq), Tol.(10 V)100° C., 12 hrstep 3" ; COC(=O)c1cc(OC)c2nc(CN3CCC(c4cccc(OCc5ccc(C#N)c6c5OCC6)n4)CC3)n(C)c2c1]

A mixture of 3 (100 mg, 570.83 μmol, 1 eq), 3A (234.31 mg, 570.83 μmol, 1 eq), CMBP (413.31 mg, 1.71 mmol, 3 eq) in Tol. (1 mL) was degassed and purged with N₂ for 3 times, and then the mixture was stirred at 100° C. for 12 hrs under N₂ atmosphere. LC-MS showed 3 was consumed completely and one main peak with desired mass was detected. The residue mixture was diluted with H₂O 20 mL and extracted with EtOAc (20 mL*3). The combined organic layers were washed with brine 20 mL, dried over Na₂SO₄, filtered and concentrated under reduced pressure to give a residue. The residue was purified by column chromatography (SiO₂, Petroleum ether/Ethyl acetate=100/1 to 0/1). 4 (150 mg, 264.25 μmol, 46.29% yield) was obtained as a white solid.

LCMS: RT=1.499 min, MS cal.: 567.25, [M+H]⁺=568.1

¹H NMR (400 MHz, CHLOROFORM-d₄) δ=7.78 (s, 1H), 7.50 (t, J=7.6 Hz, 1H), 7.39 (s, 1H), 7.35 (d, J=8.0 Hz, 1H), 7.10 (d, J=8.0 Hz, 1H), 6.73 (d, J=7.2 Hz, 1H), 6.62 (d, J=8.4 Hz, 1H), 5.37 (s, 2H), 4.72 (t, J=8.8 Hz, 2H), 4.07 (s, 3H), 3.97 (s, 6H), 3.88 (s, 2H), 3.41 (t, J=8.8 Hz, 2H), 2.97 (d, J=11.2 Hz, 2H), 2.60 (t, J=11.6 Hz, 1H), 2.28 (t, J=10.8 Hz, 2H), 1.93-1.85 (m, 2H), 1.84-1.74 (m, 2H)

### General Procedure for Preparation of Compound 283 (5):

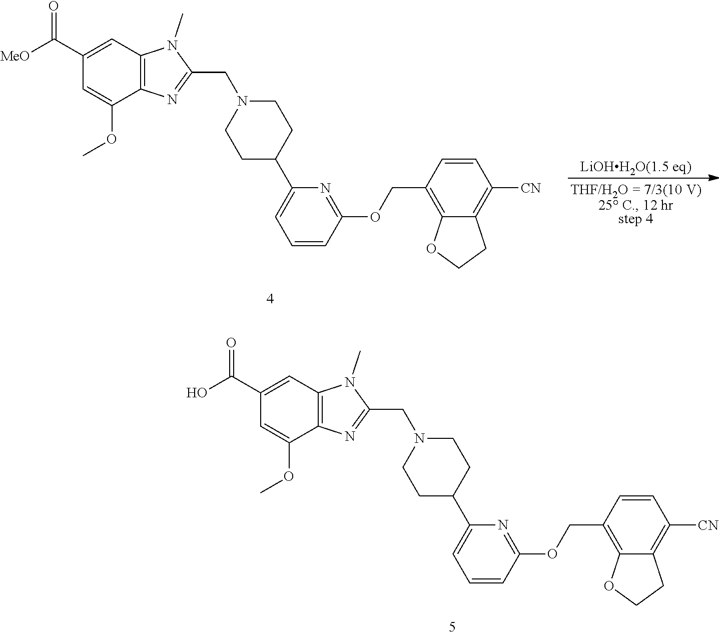

A mixture of 4 (100 mg, 176.17 μmol, 1 eq), LiOH·H₂O (11.09 mg, 264.25 μmol, 1.5 eq) in THF (0.7 mL) and H₂O (0.3 mL) was degassed and purged with N₂ for 3 times, and then the mixture was stirred at 25° C. for 12 hrs under N₂ atmosphere. HPLC showed ˜32% of 4 remained. One new peak was shown on HPLC and ˜63% of desired compound was detected. The reaction mixture was diluted with ACN 0.2 mL. The liquid was purified by prep-HPLC (column: Waters Xbridge Prep OBD C₁₈ 150*40 mm*10 um; mobile phase: [H₂O (10 mM NH₄HCO₃)-ACN]; gradient: 25%-75% B over 8.0 min). 2-((4-(6-((4-Cyano-2,3-dihydrobenzofuran-7-yl)methoxy)pyridin-2-yl)piperidin-1-yl)methyl)-4-methoxy-1-methyl-1H-benzo[d]imidazole-6-carboxylic acid (29.24 mg, 52.82 μmol, 29.98% yield, 99.89% purity) was obtained as a white solid.

HPLC: RT=1.805 min

¹H NMR (400 MHz, METHANOL-d₄) δ=7.83 (s, 1H), 7.56 (t, J=7.6 Hz, 1H), 7.45 (s, 1H), 7.33 (d, J=8.0 Hz, 1H), 7.11 (d, J=8.0 Hz, 1H), 6.80 (d, J=7.2 Hz, 1H), 6.62 (d, J=8.0 Hz, 1H), 5.34 (s, 2H), 4.70 (t, J=8.8 Hz, 2H), 4.03 (s, 3H), 3.97 (s, 3H), 3.89 (s, 2H), 3.36 (t, J=8.8 Hz, 2H), 3.04 (br d, J=11.2 Hz, 2H), 2.67-2.57 (m, 1H), 2.38-2.27 (m, 2H), 1.88-1.79 (m, 4H)

### Example A234—Preparation of Compound 282. 2-(4-(6-((4-Chloro-2-fluorobenzyl)oxy)pyridin-2-yl)-2,5-difluorobenzyl)-4-methoxy-1-methyl-1H-benzo[d]imidazole-6-carboxylic acid (Compound 282) (6)

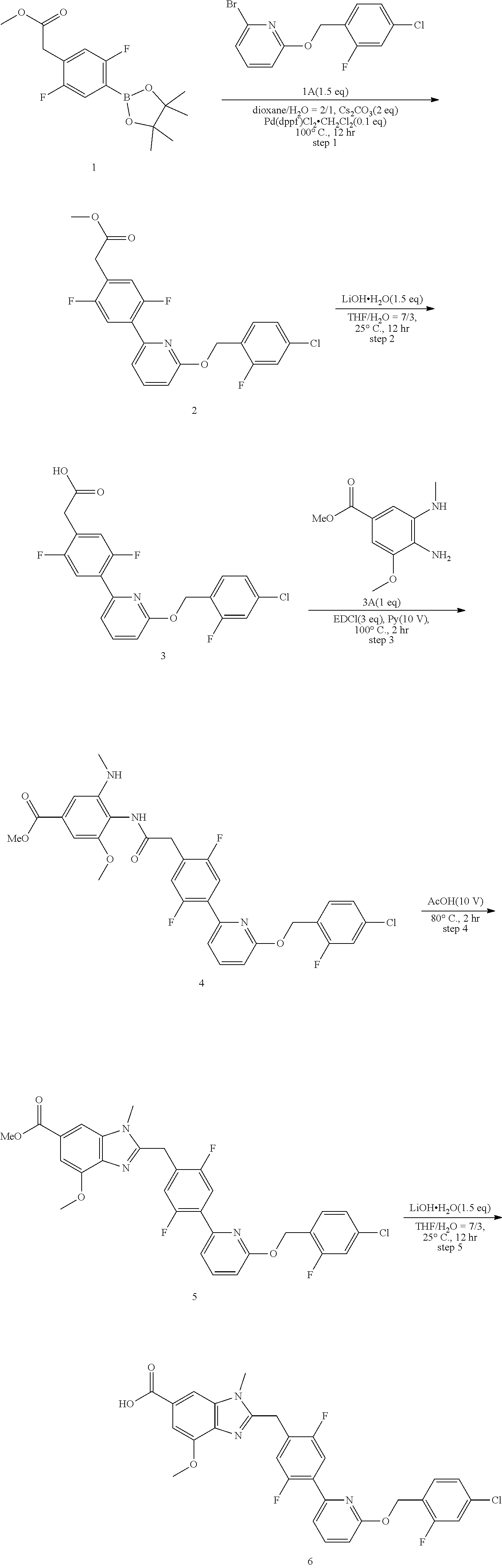

### General Procedure for Preparation of Intermediate 2:

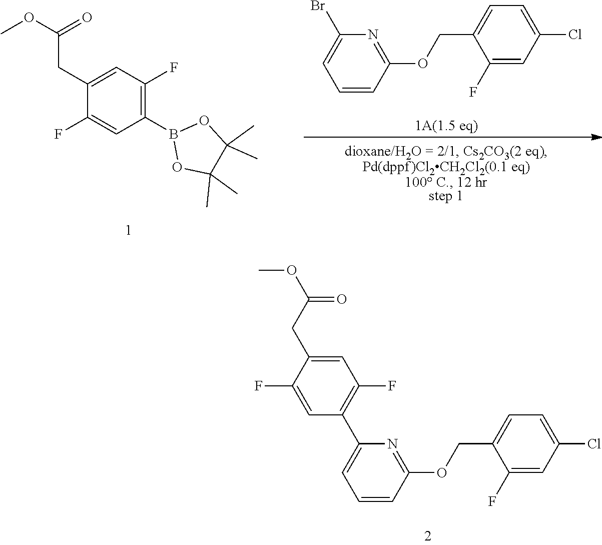

A mixture of 1 (1.3 g, 916.33 μmol, 1 eq), 1A (435.10 mg, 1.37 mmol, 1.5 eq), Cs₂CO₃ (597.11 mg, 1.83 mmol, 2 eq), Pd(dppf)Cl₂·CH₂Cl₂ (74.83 mg, 91.63 μmol, 0.1 eq) in dioxane (10 mL) and H₂O (5 mL) was degassed and purged with N₂ for 3 times, and then the mixture was stirred at 100° C. for 12 hr under N₂ atmosphere. LC-MS (RT=0.664 min) showed 1 was consumed completely and desired mass was detected. The reaction mixture was diluted with H₂O 10 mL and extracted with EtOAc 20 mL (10 mL*2). The combined organic layers were washed with brine 10 mL, dried over Na₂SO₄, filtered and concentrated under reduced pressure to give a residue. The residue was purified by column chromatography (SiO₂, Petroleum ether/Ethyl acetate=10/1 to 1/1). 2 (200 mg, 474.16 μmol, 51.75% yield) was obtained as a colourless oil.

LCMS: RT=0.668 min, MS cal.: 421.8, [M+H]⁺=422.0

¹H NMR (400 MHz, CHLOROFORM-d) δ=7.81 (dd, J=6.4, 10.8 Hz, 1H), 7.68 (t, J=7.6 Hz, 1H), 7.55-7.43 (m, 2H), 7.18-7.04 (m, 3H), 6.82-6.78 (m, 1H), 5.50 (s, 2H), 3.76 (s, 3H), 3.72 (s, 2H)

### General Procedure for Preparation of Intermediate 3:

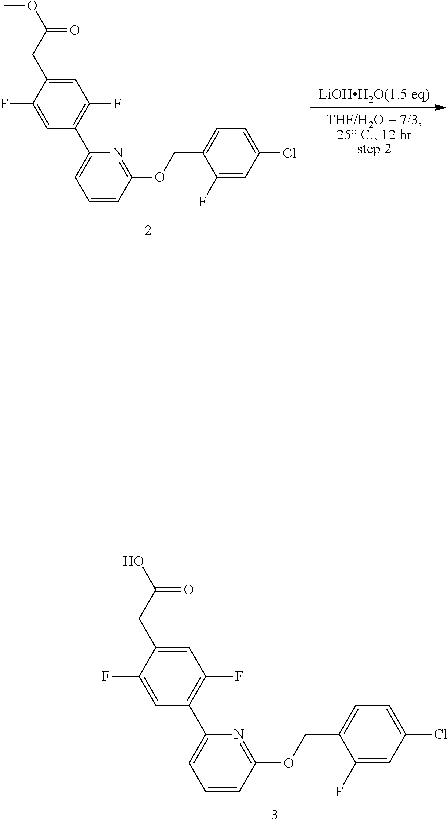

A mixture of 2 (150 mg, 355.62 μmol, 1 eq), LiOH·H₂O (22.38 mg, 533.43 μmol, 1.5 eq) in THF (1.4 mL) H₂O (0.6 mL) was degassed and purged with N₂ for 3 times, and then the mixture was stirred at 25° C. for 12 hr under N₂ atmosphere. LC-MS (RT=0.592 min) showed 2 was consumed completely and desired mass was detected. ET43596-1429 (50 mg) was combined work up. The reaction mixture was quenched with 1M Citric acid to adjust pH=5, and then diluted with H₂O 5 mL and extracted with EtOAc 10 mL (5 mL*2). The combined organic layers were washed with brine 10 mL, dried over Na₂SO₄, filtered and concentrated under reduced pressure to give a residue. 3 (160 mg, crude) was obtained as a white solid.

LCMS: RT=0.598 min, MS cal.: 407.8, [M+H]⁺=408.0

¹H NMR (400 MHz, DMSO-d₆) δ=7.84 (t, J=8.0 Hz, 1H), 7.68 (dd, J=6.4, 10.4 Hz, 1H), 7.59 (t, J=8.4 Hz, 1H), 7.51-7.44 (m, 2H), 7.34-7.24 (m, 2H), 6.90 (d, J=8.4 Hz, 1H), 5.48 (s, 2H), 3.42 (br s, 2H)

### General Procedure for Preparation of Intermediate 4 & 5:

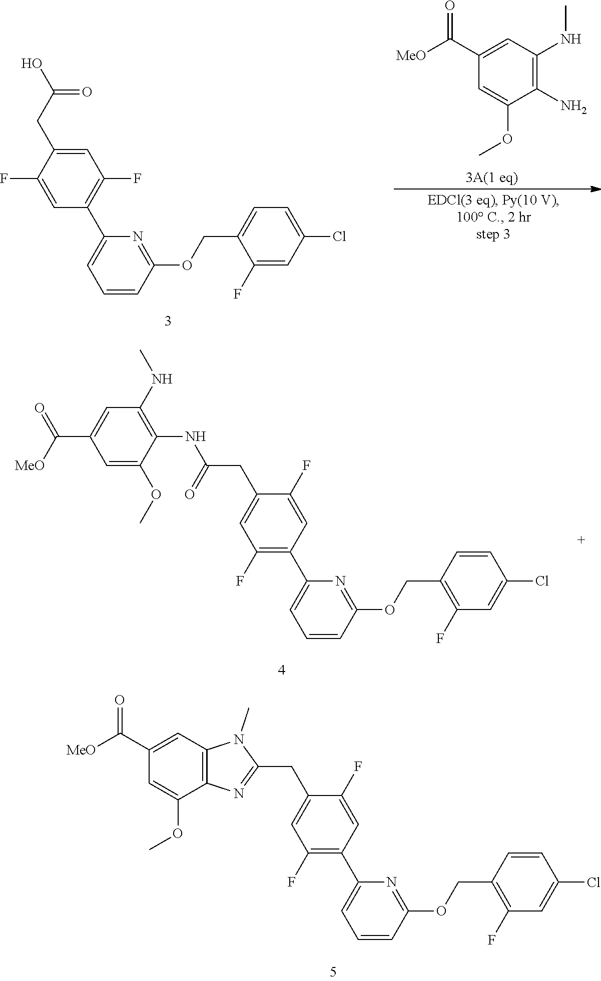 [SMILES: O=C(O)Cc1cc(F)c(-c2cccc(OCc3ccc(Cl)cc3F)n2)cc1F ; CNc1cc(C(=O)OC)cc(OC)c1N ; CNc1cc(C(=O)OC)cc(OC)c1NC(=O)Cc1cc(F)c(-c2cccc(OCc3ccc(Cl)cc3F)n2)cc1F ; COC(=O)c1cc(OC)c2nc(Cc3cc(F)c(-c4cccc(OCc5ccc(Cl)cc5F)n4)cc3F)n(C)c2c1]

A mixture of 3 (100 mg, 245.24 μmol, 1 eq), 3A (51.56 mg, 245.24 μmol, 1 eq), EDCI (141.04 mg, 735.71 μmol, 3 eq) in Py (2 mL) was degassed and purged with N₂ for 3 times, and then the mixture was stirred at 100° C. for 2 hr under N₂ atmosphere. LC-MS (RT₄=1.667 min, RT₅=1.710 min) showed 3 was consumed completely and desired mass was detected. The reaction was quenched with 1M FA to adjust pH=5 at 0° C. and extracted with EtOAc 10*5 mL. The combined organic layers were washed with brine 10 mL, dried over Na₂SO₄, filtered, and concentrated under reduced pressure to give a residue. 4 & 5 (170 mg, crude, ˜1:1) were obtained as a yellow oil.

LCMS: RT5=1.710 min, MS cal.: 582.0, [M+H]⁺=582.0

### General Procedure for Preparation of Intermediate 5:

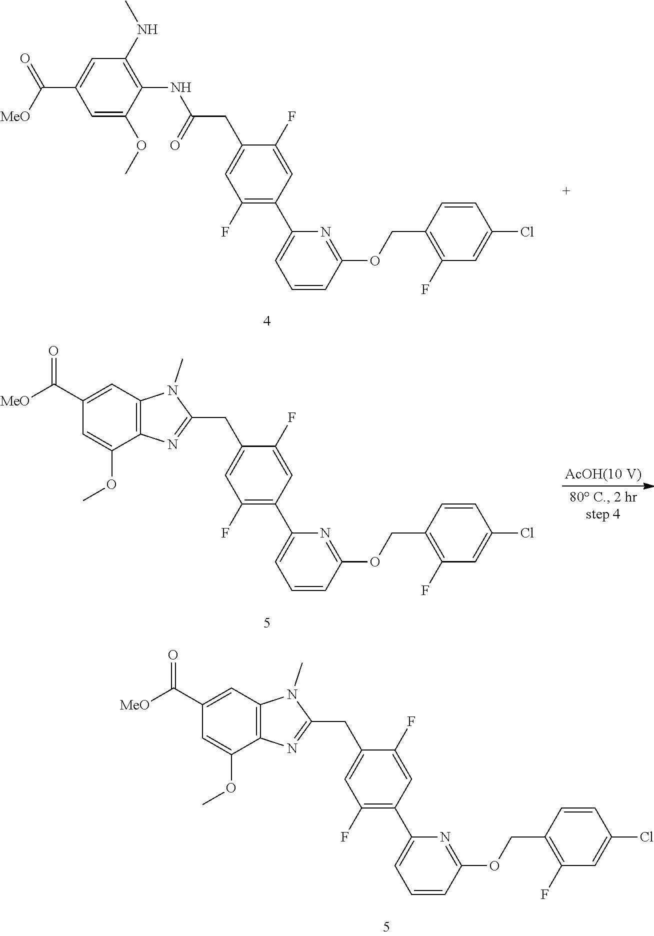 [SMILES: CNc1cc(C(=O)OC)cc(OC)c1NC(=O)Cc1cc(F)c(-c2cccc(OCc3ccc(Cl)cc3F)n2)cc1F ; COC(=O)c1cc(OC)c2nc(Cc3cc(F)c(-c4cccc(OCc5ccc(Cl)cc5F)n4)cc3F)n(C)c2c1 ; COC(=O)c1cc(OC)c2nc(Cc3cc(F)c(-c4cccc(OCc5ccc(Cl)cc5F)n4)cc3F)n(C)c2c1]

A mixture of 4 & 5 (170 mg, crude, ˜1:1) in AcOH (1.7 mL) was degassed and purged with N₂ for 3 times, and then the mixture was stirred at 80° C. for 2 hr under N₂ atmosphere. LC-MS (RT=0.581 min) showed 4 was consumed completely and desired mass was detected. The reaction mixture was concentrated under reduced pressure to give a residue. The mixture was quenched with saturated NaHCO₃ solution to adjust pH=7 at 0° C. and extracted with EtOAc 20 mL (10 mL*2). The combined organic layers were washed with brine 10 mL, dried over Na₂SO₄, filtered, and concentrated under reduced pressure to give a residue. The residue was purified by column chromatography (SiO₂, Petroleum ether/Ethyl acetate=10/1 to 0/1). 5 (65 mg, crude) was obtained as a yellow solid.

LCMS: RT=0.571 min, MS cal.: 582.0, [M+H]⁺=582.1

¹H NMR (400 MHz, CHLOROFORM-d) δ=7.76 (dd, J=6.4, 10.8 Hz, 1H), 7.69-7.65 (m, 1H), 7.58 (t, J=8.0 Hz, 1H), 7.43-7.32 (m, 3H), 7.08 (d, J=8.4 Hz, 2H), 6.96 (br d, J=4.4 Hz, 1H), 6.71 (d, J=8.4 Hz, 1H), 5.42 (s, 2H), 4.33 (s, 2H), 4.04-4.00 (m, 3H), 3.89 (s, 3H), 3.67 (s, 3H)

### General Procedure for Preparation of Compound 282 (6):

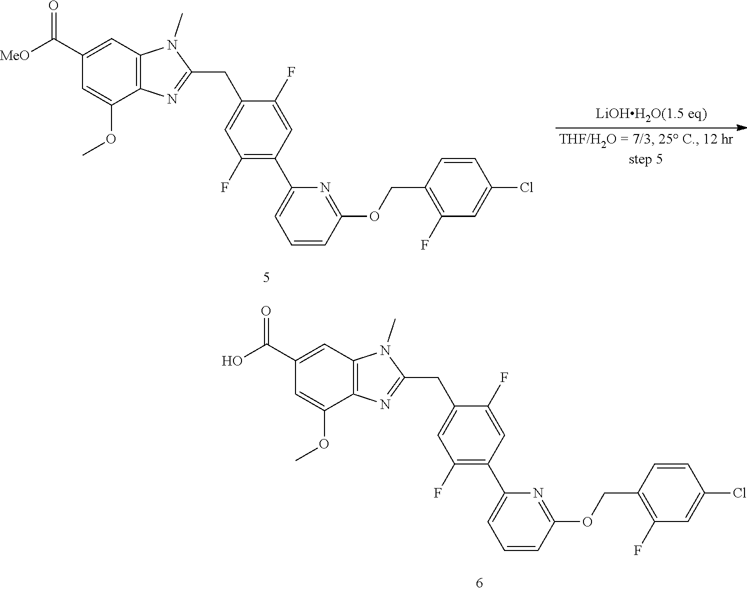 [SMILES: COC(=O)c1cc(OC)c2nc(Cc3cc(F)c(-c4cccc(OCc5ccc(Cl)cc5F)n4)cc3F)n(C)c2c1 ; COc1cc(C(=O)O)cc2c1nc(Cc1cc(F)c(-c3cccc(OCc4ccc(Cl)cc4F)n3)cc1F)n2C]

A mixture of 5 (60 mg, 103.10 μmol, 1 eq), LiOH·H₂O (6.49 mg, 154.65 μmol, 1.5 eq) in THF (0.7 mL) and H₂O (0.3 mL) was degassed and purged with N₂ for 3 times, and then the mixture was stirred at 25° C. for 12 hr under N₂ atmosphere. LC-MS (RT=0.534 min) showed 5 was consumed completely and desired mass was detected. The reaction mixture was concentrated to give a residue. The crude product was purified by prep-HPLC (column: Phenomenex Luna C18 100*30 mm*3 um; mobile phase: [H₂O (0.2% FA)-ACN]; gradient: 35%-75% B over 8.0 min). 2-(4-(6-((4-Chloro-2-fluorobenzyl)oxy)pyridin-2-yl)-2,5-difluorobenzyl)-4-methoxy-1-methyl-1H-benzo[d]imidazole-6-carboxylic acid (14.31 mg, 25.13 μmol, 24.37% yield, 99.72% purity) was obtained as a yellow solid.

LCMS: RT=2.597 min, MS cal.: 567.9, [M+H]⁺=568.1

HPLC: RT=14.053 min, purity: 99.72%

¹H NMR (400 MHz, DMSO-d₆) δ=13.03-12.70 (m, 1H), 7.89-7.78 (m, 3H), 7.60 (t, J=8.4 Hz, 1H), 7.53-7.47 (m, 2H), 7.38-7.30 (m, 2H), 7.26 (s, 1H), 6.94 (d, J=8.4 Hz, 1H), 5.50 (s, 2H), 4.39 (s, 2H), 3.90 (s, 3H), 3.86 (s, 3H)

### Example A235—Preparation of Compound 281. 5-(2-((4-(3-((4-Chloro-2-fluorobenzyl)oxy)phenyl)piperidin-1-yl)methyl)-4-(difluoromethoxy)-1-methyl-1H-benzo[d]imidazol-6-yl)-1,2,5-thiadiazolidin-3-one 1,1-dioxide (Compound 281) (15)

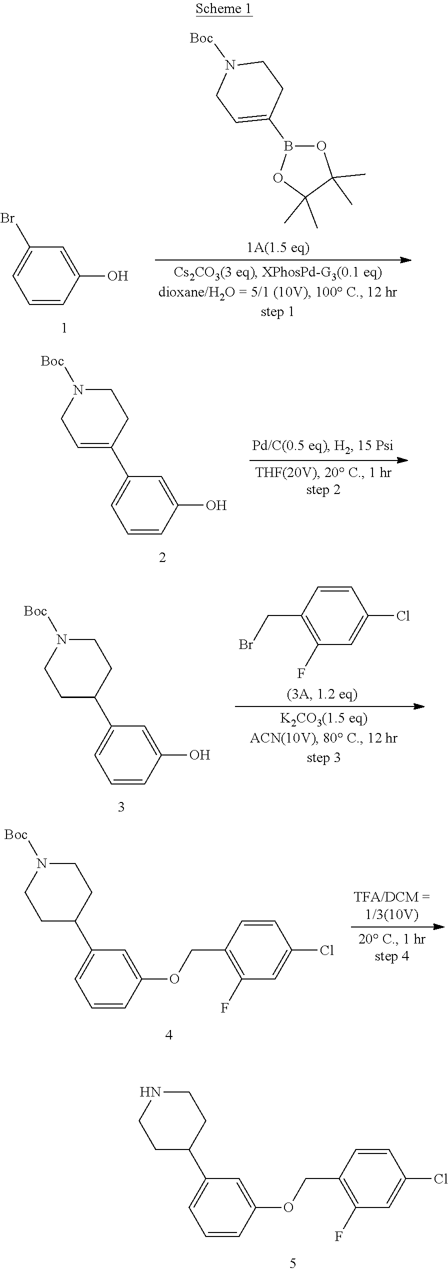

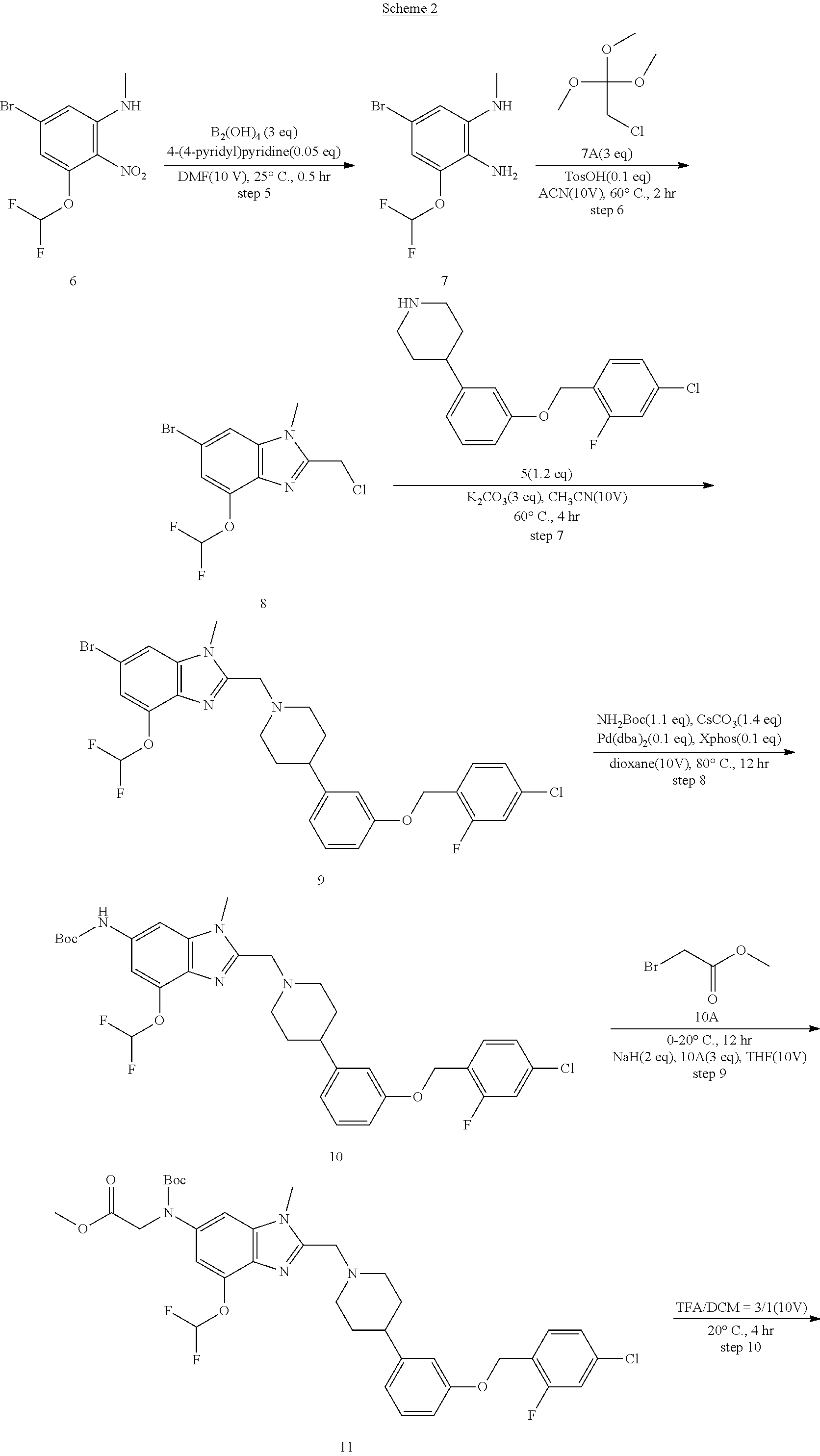

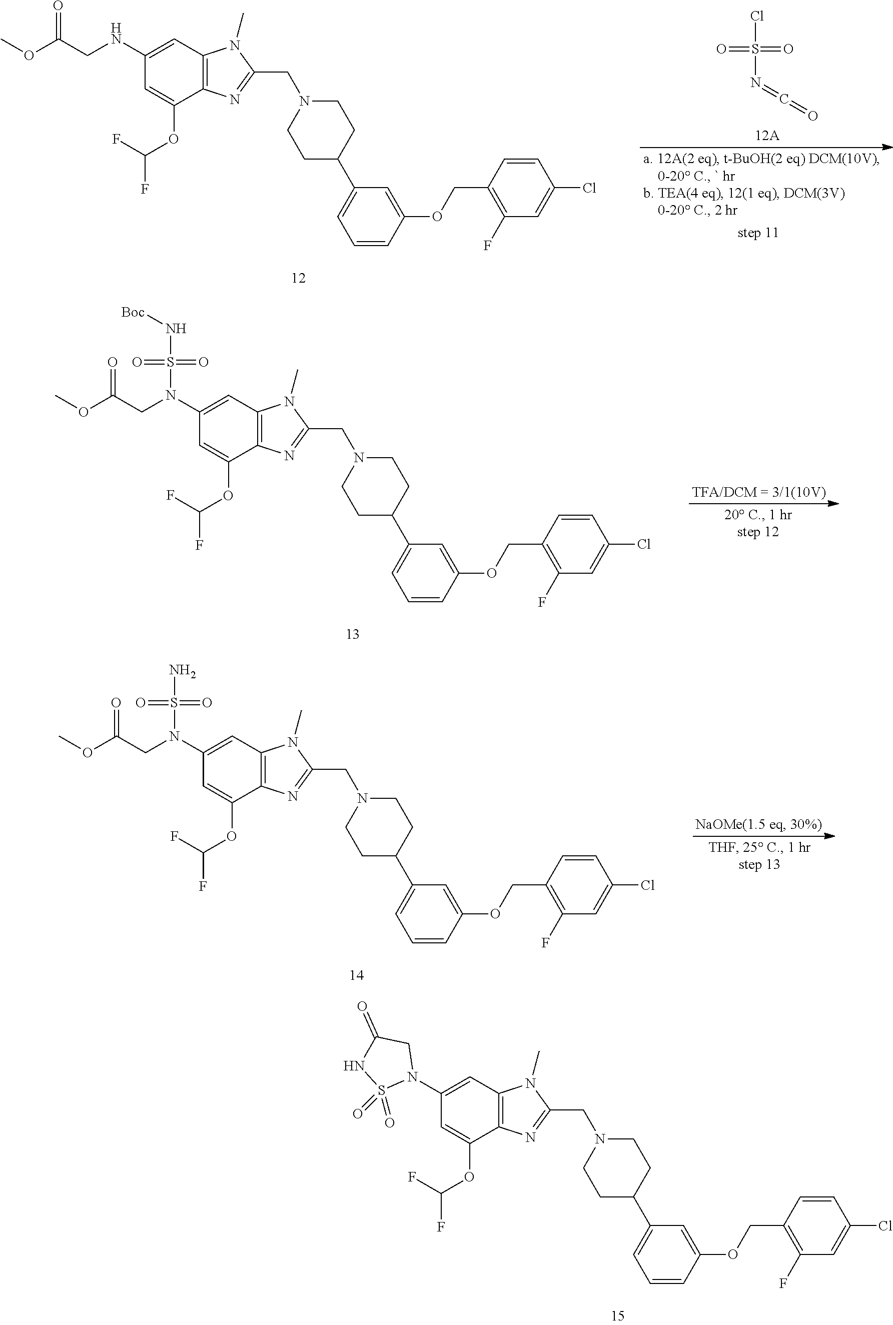 [SMILES: COC(=O)CNc1cc(OC(F)F)c2nc(CN3CCC(c4cccc(OCc5ccc(Cl)cc5F)c4)CC3)n(C)c2c1 ; O=C=NS(=O)(=O)Cl ; COC(=O)CN(c1cc(OC(F)F)c2nc(CN3CCC(c4cccc(OCc5ccc(Cl)cc5F)c4)CC3)n(C)c2c1)S(=O)(=O)NC(=O)OC(C)(C)C ; COC(=O)CN(c1cc(OC(F)F)c2nc(CN3CCC(c4cccc(OCc5ccc(Cl)cc5F)c4)CC3)n(C)c2c1)S(N)(=O)=O ; Cn1c(CN2CCC(c3cccc(OCc4ccc(Cl)cc4F)c3)CC2)nc2c(OC(F)F)cc(N3CC(=O)NS3(=O)=O)cc21]

### General Procedure for Preparation of Scheme 1, Intermediate 2:

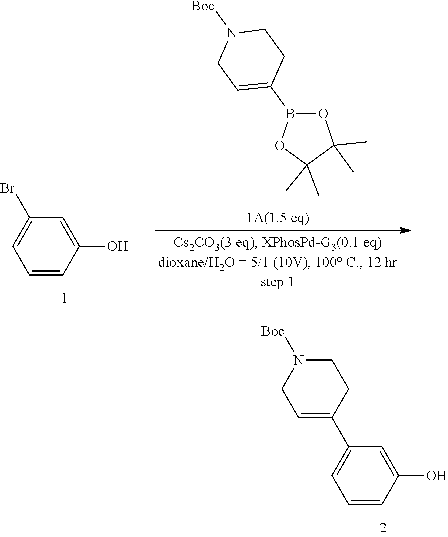 [SMILES: Oc1cccc(Br)c1 ; CC(C)(C)OC(=O)N1CC=C(B2OC(C)(C)C(C)(C)O2)CC1 ; CC(C)(C)OC(=O)N1CC=C(c2cccc(O)c2)CC1]

To a solution of 1 (1 g, 5.78 mmol, 1 eq), 1A (2.68 g, 8.67 mmol, 1.5 eq) in dioxane (10 mL) H₂O (2 mL) was added Cs₂CO₃ (5.65 g, 17.34 mmol, 3 eq) and XPhos Pd G₃ (489.25 mg, 578.01 μmol, 0.1 eq). The mixture was stirred at 100° C. for 12 hr. LC-MS showed 1 was consumed completely and one main peak with desired mass was detected. The mixture was diluted with H₂O (15 mL), extracted with EtOAc (15 mL*3). The combined organic layer was washed with 10 mL brine, dried over Na₂SO₄, filtered and the filtrate was concentrated to give a crude product The residue was purified by column chromatography (SiO₂, Petroleum ether/Ethyl acetate=10/1). 2 (1.7 g, crude) was obtained as a brown solid.

LCMS: RT=1.280 min, MS cal.: 275.1, 276.1, [M−H]⁺=274.0, 275.0

¹H NMR (400 MHz, CHLOROFORM-d) δ=7.19 (t, J=7.8 Hz, 1H), 6.93 (d, J=7.6 Hz, 1H), 6.88 (s, 1H), 6.75 (d, J=8.0 Hz, 1H), 6.02 (s, 1H), 4.08 (d, J=1.5 Hz, 2H), 3.64 (t, J=5.6 Hz, 2H), 2.55-2.47 (m, 2H), 1.51 (s, 9H)

### General Procedure for Preparation of Scheme 1, Intermediate 3:

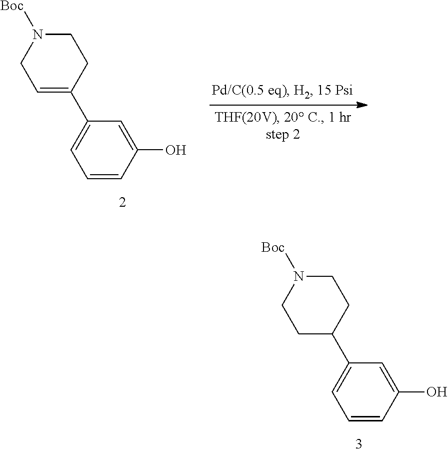 [SMILES: CC(C)(C)OC(=O)N1CC=C(c2cccc(O)c2)CC1 ; CC(C)(C)OC(=O)N1CCC(c2cccc(O)c2)CC1]

Equip a 250 mL round bottom flask. Pd/C (328.53 mg) in THF (17 mL) was added to the reaction mixture at 20° C. under N₂ atmosphere. Then 2 (1.7 g, 6.17 mmol, 1 eq) in THF (17 mL) was added to the mixture at 20° C. After the addition, the suspension was degassed and purged with H₂ for 3 times, and then the mixture was stirred under H₂ (15 Psi) atmosphere at 20° C. for 1 hr. LC-MS showed 2 was consumed completely and one main peak with desired mass was detected. The reaction mixture was filtered through celite pad. The filter cake was rinsed with THF (50 mL*3), and the filtrate was concentrated under reduced pressure to give a residue. 3 (1.7 g, 6.13 mmol, 99% yield) was obtained as a brown oil.

LCMS: RT=1.278 min, MS cal.: 277.1, 278.1, [M−H]⁺=276.0, 277.0

¹H NMR (400 MHz, CHLOROFORM-d) δ=7.19-7.13 (m, 1H), 6.75 (d, J=7.6 Hz, 1H), 6.72-6.38 (m, 2H), 4.24 (d, J=13.2 Hz, 2H), 2.80 (td, J=13.0, 2.0 Hz, 2H), 2.60 (tt, J=12.0, 3.6 Hz, 1H), 1.82 (d, J=13.6 Hz, 2H), 1.66-1.57 (m, 2H), 1.50 (s, 9H)

### General Procedure for Preparation of Intermediate 4:

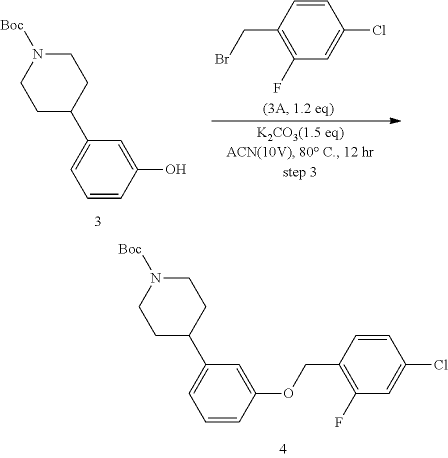 [SMILES: CC(C)(C)OC(=O)N1CCC(c2cccc(O)c2)CC1 ; Fc1cc(Cl)ccc1CBr ; CC(C)(C)OC(=O)N1CCC(c2cccc(OCc3ccc(Cl)cc3F)c2)CC1]

To a solution of 3 (6.8 g, 24.52 mmol, 1 eq) 3A (6.03 g, 26.97 mmol, 1.1 eq) in ACN (68 mL) was added K₂CO₃ (6.78 g, 49.03 mmol, 2 eq). The mixture was stirred at 60° C. for 4 hr. LC-MS showed 3 was consumed completely and the desired mass was detected as a main peak. The mixture was diluted with H₂O (35 mL), extracted with EtOAc (35 mL*3). The combined organic layer was washed with 30 mL brine, dried over Na₂SO₄, filtered and the filtrate was concentrated to give a crude product. 4 (8.7 g, 20.72 mmol, 85% yield) was obtained as a white solid.

LCMS: RT=1.787 min, MS cal.: 419.2, 421.2, [M−H]⁺=418.0, 420.0

¹H NMR (400 MHz, CHLOROFORM-d) δ=7.46 (t, J=8.0 Hz, 1H), 7.27-7.22 (m, 1H), 7.18 (dd, J=8.2, 2.0 Hz, 1H), 7.14 (dd, J=10.0, 2.0 Hz, 1H), 6.86-6.80 (m, 3H), 5.09 (s, 2H), 4.25 (d, J=13.4 Hz, 2H), 2.80 (td, J=13.2, 2.4 Hz, 2H), 2.63 (tt, J=12.0, 3.6 Hz, 1H), 1.83 (d, J=13.2 Hz, 2H), 1.68-1.55 (m, 2H), 1.49 (s, 9H)

### General Procedure for Preparation of Scheme 1, Intermediate 5:

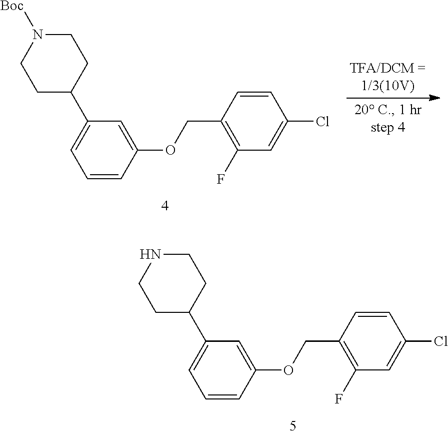 [SMILES: CC(C)(C)OC(=O)N1CCC(c2cccc(OCc3ccc(Cl)cc3F)c2)CC1 ; Fc1cc(Cl)ccc1COc1cccc(C2CCNCC2)c1]

To a solution of 4 (460 mg, 1.10 mmol, 1 eq) in DCM (6 mL) was added TFA (2 mL). The mixture was stirred at 20° C. for 1 hr. LC-MS showed 4 was consumed completely and the desired mass was detected as a main peak. The mixture was added a.q. Na₂CO₃ 15 mL, then diluted with H₂O (15 mL), extracted with DCM (15 mL*3). The combined organic layer was washed with 10 mL brine, dried over Na₂SO₄, filtered and the filtrate was concentrated to give a crude product. 5 (350 mg, 1.09 mmol, 99.91% yield) was obtained as a white solid.

LCMS: RT=0.400 min, MS cal.: 319.1, 321.1, [M+H]⁺=320.0, 322.0

¹H NMR (400 MHz, CHLOROFORM-d) δ=7.47 (t, J=8.0 Hz, 1H), 7.24 (t, J=8.2 Hz, 1H), 7.17 (dd, J=8.2, 2.0 Hz, 1H), 7.13 (dd, J=10.0, 2.0 Hz, 1H), 6.90-6.84 (m, 2H), 6.83-6.78 (m, 1H), 5.09 (s, 2H), 3.23 (d, J=12.0 Hz, 2H), 2.85-2.71 (m, 3H), 2.62 (tt, J=12.0, 3.6 Hz, 1H), 1.86 (d, J=12.0 Hz, 2H), 1.75-1.62 (m, 2H)

### General Procedure for Preparation of Scheme 2, Intermediate 7:

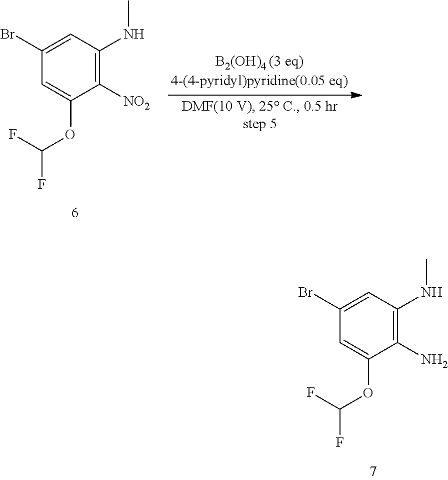 [SMILES: CNc1cc(Br)cc(OC(F)F)c1[N+](=O)[O-] ; CNc1cc(Br)cc(OC(F)F)c1N]

To a solution of 6 (5 g, 16.83 mmol, 1 eq) in DMF (50 mL) was added 4-(4-pyridyl)pyridine (13.14 mg, 84.16 μmol, 0.005 eq) and hypoboric acid (4.53 g, 50.50 mmol, 3 eq). The mixture was stirred at 25° C. for 1 hr. LC-MS showed 6 was consumed completely and one main peak with desired mass was detected. The mixture was diluted with H₂O (55 mL), extracted with EtOAc (35 mL*3). The combined organic layer was washed with 30 mL brine, dried over Na₂SO₄, filtered and the filtrate was concentrated to give a crude product. The residue was purified by column chromatography (SiO₂, Petroleum ether/Ethyl acetate=10/1). 7 (3.3 g, 12.36 mmol, 73% yield) was obtained as a brown oil.

LCMS: RT=1.226 min, MS cal.: 266.0, 268.0, [M+H]⁺=266.9, 268.9

¹H NMR (400 MHz, CHLOROFORM-d) δ=6.74-6.71 (m, 1H), 6.63 (d, J=2.0 Hz, 1H), 6.46 (t, J=74.0 Hz, 1H), 3.63-3.21 (m, 3H), 2.86 (s, 3H)

### General Procedure for Preparation of Scheme 2, Intermediate 8:

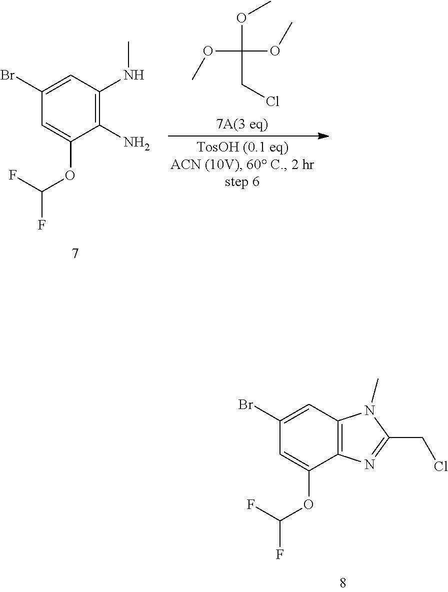

To a solution of 7 (2.8 g, 10.48 mmol, 1 eq) in ACN (28 mL) was added TosOH (180.54 mg, 1.05 mmol, 0.1 eq) and 7A (4.86 g, 31.45 mmol, 4.24 mL, 3 eq). The mixture was stirred at 60° C. for 2 hr. LC-MS showed 7 was consumed completely and one main peak with desired mass was detected. The mixture was diluted with H₂O (55 mL), extracted with EtOAc (35 mL*3). The combined organic layer was washed with 30 mL brine, dried over Na₂SO₄, filtered and the filtrate was concentrated to give a crude product. The residue was purified by column chromatography (SiO₂, Petroleum ether/Ethyl acetate-3/1) 8 (3.1 g, 9.52 mmol, 91% yield) was obtained as a brown solid.

LCMS: RT=0.505 min, MS cal.: 323.9, 325.9, [M+H]⁺=324.9, 326.9

¹H NMR (400 MHz, CHLOROFORM-d) δ=7.36 (s, 1H), 7.34 (t, J=74.0 Hz, 1H), 7.18 (s, 1H), 4.82 (s, 2H), 3.85 (s, 3H)

### General Procedure for Preparation of Scheme 2, Intermediate 9:

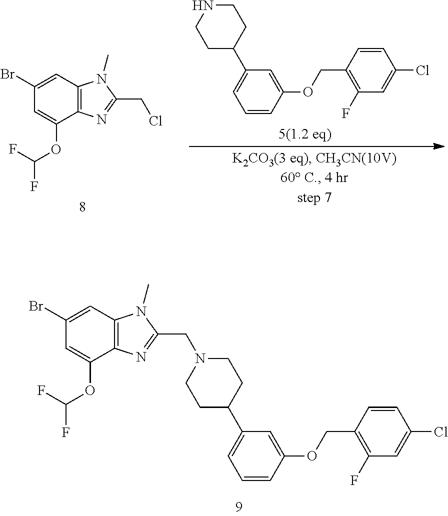 [SMILES: Cn1c(CCl)nc2c(OC(F)F)cc(Br)cc21 ; Fc1cc(Cl)ccc1COc1cccc(C2CCNCC2)c1 ; Cn1c(CN2CCC(c3cccc(OCc4ccc(Cl)cc4F)c3)CC2)nc2c(OC(F)F)cc(Br)cc21]

To a solution of 8 (300 mg, 921.55 μmol, 1 eq) 5 (324.18 mg, 1.01 mmol, 1.1 eq) in ACN (3 mL) was added K₂CO₃ (382.09 mg, 2.76 mmol, 3 eq). The mixture was stirred at 60° C. for 4 hr. LC-MS showed 8 was consumed completely and one main peak with desired mass was detected. The mixture was diluted with H₂O (10 mL), extracted with EtOAc (15 mL*3). The combined organic layer was washed with 30 mL brine, dried over Na₂SO₄, filtered and the filtrate was concentrated to give a crude product. The residue was purified by column chromatography (SiO₂, Petroleum ether/Ethyl acetate=1/1). 9 (430 mg, 706.22 μmol, 77% yield) was obtained as a brown oil.

LCMS: RT=1.873 min, MS cal.: 607.1, 609.1, [M+H]⁺=608.0, 610.0

¹H NMR (400 MHz, CHLOROFORM-d) δ=7.54-7.48 (m, 1H), 7.47-7.41 (m, 1H), 7.38-7.35 (m, 1H), 7.36 (t, J=74.0 Hz, 1H), 7.16 (d, J=1.2 Hz, 1H), 7.14-7.08 (m, 2H), 6.73 (d, J=7.2 Hz, 1H), 6.61 (d, J=8.2 Hz, 1H), 3.91 (s, 3H), 2.94 (d, J=11.6 Hz, 2H), 2.61 (tt, J=11.6, 4.0 Hz, 1H), 2.28 (td, J=11.6, 2.4 Hz, 2H), 1.92-1.74 (m, 4H)

### General Procedure for Preparation of Scheme 2, Intermediate 10:

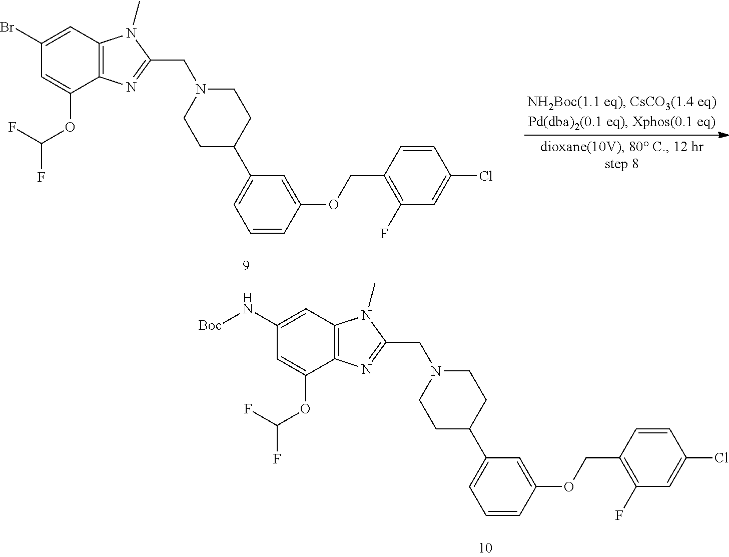 [SMILES: Cn1c(CN2CCC(c3cccc(OCc4ccc(Cl)cc4F)c3)CC2)nc2c(OC(F)F)cc(Br)cc21 ; Cn1c(CN2CCC(c3cccc(OCc4ccc(Cl)cc4F)c3)CC2)nc2c(OC(F)F)cc(NC(=O)OC(C)(C)C)cc21]

To a solution of 9 (1 g, 1.64 mmol, 1 eq), tert-butyl carbamate (211.64 mg, 1.81 mmol, 1.1 eq) in dioxane (10 mL) was added Cs₂CO₃ (749.16 mg, 2.30 mmol, 1.4 eq) and XPhos (78.29 mg, 164.24 μmol, 0.1 eq) Pd(dba)₂ (94.44 mg, 164.24 μmol, 0.1 eq). The mixture was stirred at 80° C. for 12 hr. LC-MS showed 9 was consumed completely and one main peak with desired mass was detected. The mixture was diluted with H₂O (10 mL), extracted with EtOAc (15 mL*3). The combined organic layer was washed with 30 mL brine, dried over Na₂SO₄, filtered and the filtrate was concentrated to give a crude product. The residue was purified by prep-HPLC (column: Waters Xbridge Prep OBD C18 150*40 mm*10 um; mobile phase: [H₂O (10 mM NH₄HCO₃)-ACN]; gradient: 75%-100% B over 8.0 min. 10 (550 mg, 852.57 μmol, 52% yield) was obtained as a white solid.

LCMS: RT=1.817 min, MS cal.: 644.2, 645.2, [M+H]⁺=645.1, 646.1

¹H NMR (400 MHz, METHANOL-d₄) δ=7.59 (s, 1H), 7.53-7.48 (m, 1H), 7.26-7.16 (m, 3H), 7.15 (t, J=74.0 Hz, 1H), 7.01 (s, 1H), 6.87-6.83 (m, 2H), 6.82-6.78 (m, 1H), 5.09 (s, 2H), 3.90 (s, 3H), 3.83 (s, 2H), 3.01 (d, J=12.0 Hz, 2H), 2.58-2.48 (m, 1H), 2.33-2.23 (m, 2H), 1.86-1.68 (m, 5H), 1.54 (s, 9H)

### General Procedure for Preparation of Scheme 2, Intermediate 11:

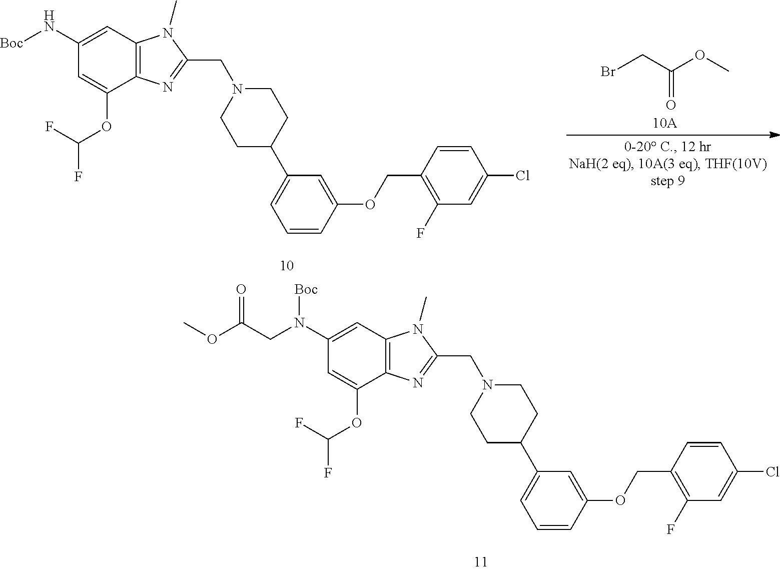

Equip a 50 mL three-neck bottom flask.

Then 10 (1.1 g, 1.71 mmol, 1 eq) in THF (11 mL) at 20° C. At 0° C. (inner temperature) NaH (136.40 mg, 3.41 mmol, 60% purity, 2 eq) was added to the mixture at 0° C. under N₂ atmosphere, the mixture was stirred at 0° C. for 0.5 hr. Then was added 10A (782.53 mg, 5.12 mmol, 484.24 μL, 3 eq) to the mixture. After the addition, the suspension was degassed and purged with N₂ for 3 times, and then the mixture was stirred at 20° C. for 12 hr. TLC indicated 10 was consumed completely and one new spot formed. The reaction was clean according to TLC. The mixture was diluted with H₂O (10 mL), extracted with EtOAc (15 mL*3). The combined organic layer was washed with 30 mL brine, dried over Na₂SO₄, filtered and the filtrate was concentrated to give a crude product. The residue was purified by column chromatography (SiO₂, Petroleum ether/Ethyl acetate=5/1). 11 (800 mg, 1.12 mmol, 65% yield) was obtained as a yellow solid.

LCMS: RT=1.794 min, MS cal.: 716.3, 717.3, [M+H]⁺=717.2, 718.2

¹H NMR (400 MHz, METHANOL-d₄) δ=7.52-7.46 (m, 1H), 7.38 (s, 1H), 7.25-7.15 (m, 1H), 7.26-7.14 (m, 2H), 7.21 (t, J=74.0 Hz, 1H), 7.03 (s, 1H), 6.86-6.82 (m, 2H), 6.82-6.77 (m, 1H), 5.08 (s, 2H), 4.38 (s, 2H), 3.93 (s, 3H), 3.86 (s, 2H), 3.78 (s, 3H), 3.00 (d, J=11.4 Hz, 2H), 2.58-2.47 (m, 1H), 2.33-2.24 (m, 2H), 1.84-1.68 (m, 4H), 1.44 (s, 9H)

### General Procedure for Preparation of Scheme 2, Intermediate 12:

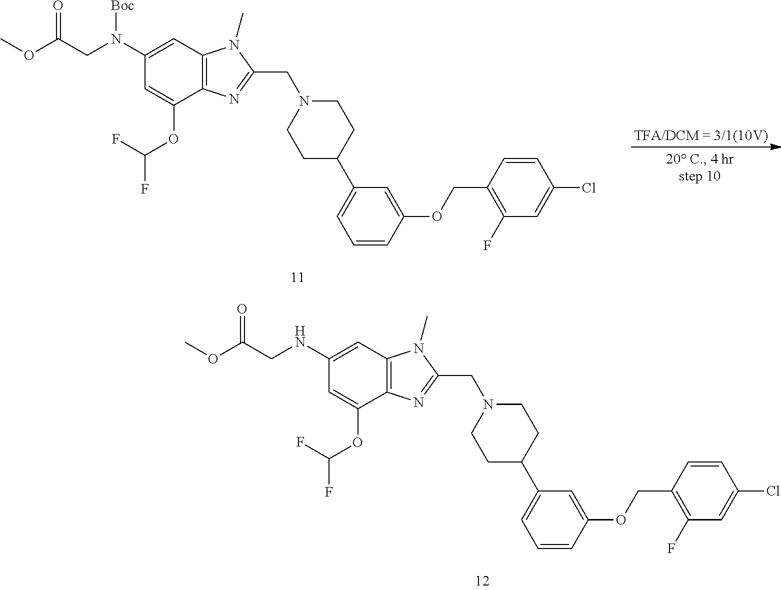 [SMILES: COC(=O)CN(C(=O)OC(C)(C)C)c1cc(OC(F)F)c2nc(CN3CCC(c4cccc(OCc5ccc(Cl)cc5F)c4)CC3)n(C)c2c1 ; COC(=O)CNc1cc(OC(F)F)c2nc(CN3CCC(c4cccc(OCc5ccc(Cl)cc5F)c4)CC3)n(C)c2c1]

To a solution of 11 (800 mg, 1.12 mmol, 1 eq) in DCM (9 mL) was added TFA (3 mL). The mixture was stirred at 20° C. for 4 hr. LC-MS showed 11 was consumed completely and one main peak with desired mass was detected. The mixture was added a.q. Na₂CO₃ 15 mL, diluted with H₂O (15 mL), extracted with DCM (15 mL*3). The combined organic layer was washed with 10 mL brine, dried over Na₂SO₄, filtered and the filtrate was concentrated to give a crude product. 12 (650 mg, 1.05 mmol, 94% yield) was obtained as a white solid.

LCMS: RT=1.626 min, MS cal.: 616.2, 617.2, [M+H]⁺=617.1, 618.1

¹H NMR (400 MHz, METHANOL-d₄) δ=7.53-7.47 (m, 1H), 7.26-7.20 (m, 2H), 7.20-7.16 (m, 1H), 7.10 (t, J=74.8 Hz, 1H), 6.88-6.77 (m, 3H), 6.46 (s, 1H), 6.42 (d, J=1.8 Hz, 1H), 5.08 (s, 2H), 4.01 (s, 2H), 3.83 (s, 3H), 3.77 (s, 2H), 3.75 (s, 3H), 3.00 (d, J=11.2 Hz, 2H), 2.52 (tt, J=11.2, 3.8 Hz, 1H), 2.30-2.20 (m, 2H), 1.84-1.71 (m, 4H)

### General Procedure for Preparation of Scheme 2, Intermediate 13:

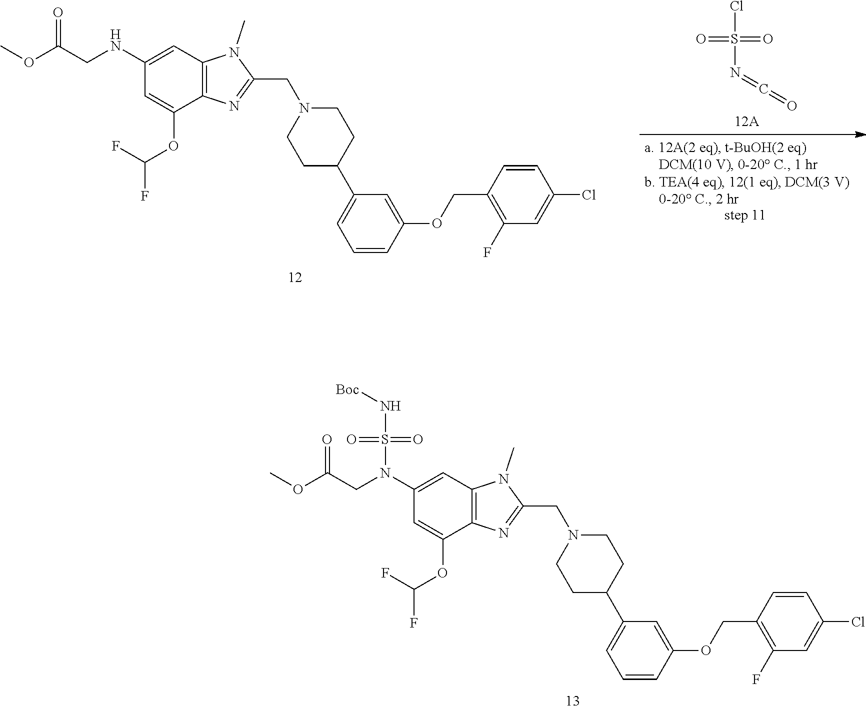 [SMILES: COC(=O)CNc1cc(OC(F)F)c2nc(CN3CCC(c4cccc(OCc5ccc(Cl)cc5F)c4)CC3)n(C)c2c1 ; O=C=NS(=O)(=O)Cl ; COC(=O)CN(c1cc(OC(F)F)c2nc(CN3CCC(c4cccc(OCc5ccc(Cl)cc5F)c4)CC3)n(C)c2c1)S(=O)(=O)NC(=O)OC(C)(C)C]

To a solution of 12A (275.24 mg, 1.94 mmol, 169.28 μL, 2 eq) in DCM (3 mL) at 0° C. was added t-BuOH (144.15 mg, 1.94 mmol, 185.99 μL, 2 eq). The mixture was stirred at 20° C. for 1 hr. The reaction was recooled to 0° C. and treated with a solution of 12 (300 mg, 486.18 μmol, 0.5 eq) and TEA (393.57 mg, 3.89 mmol, 541.36 μL, 4 eq) as a solution in DCM (3 mL). The mixture was stirred at 20° C. for 2 hr. LC-MS showed 12 was consumed completely and one main peak with desired mass was detected. The reaction mixture was concentrated under reduced pressure to remove DCM. The residue was purified by column chromatography (SiO₂, Petroleum ether/Ethyl acetate=0/1). 13 (400 mg, 502.35 μmol, 52% yield) was obtained as a brown solid.

LCMS: RT=1.418 min, MS cal.: 795.2, 796.2, [M−H]⁺=794.3, 795.3

¹H NMR (400 MHz, METHANOL-d₄) δ=7.58 (d, J=1.6 Hz, 1H), 7.53-7.47 (m, 1H), 7.27-7.16 (m, 3H), 7.23 (t, J=76 Hz, 1H), 7.11 (s, 1H), 6.88-6.79 (m, 3H), 5.09 (s, 2H), 4.72 (s, 2H), 3.99-3.94 (m, 3H), 3.91 (s, 2H), 3.79-3.70 (m, 3H), 3.02 (d, J=11.2 Hz, 2H), 2.59-2.50 (m, 1H), 2.39-2.28 (m, 2H), 1.86-1.71 (m, 4H), 1.50 (s, 9H)

### General Procedure for Preparation of Scheme 2, Intermediate 14:

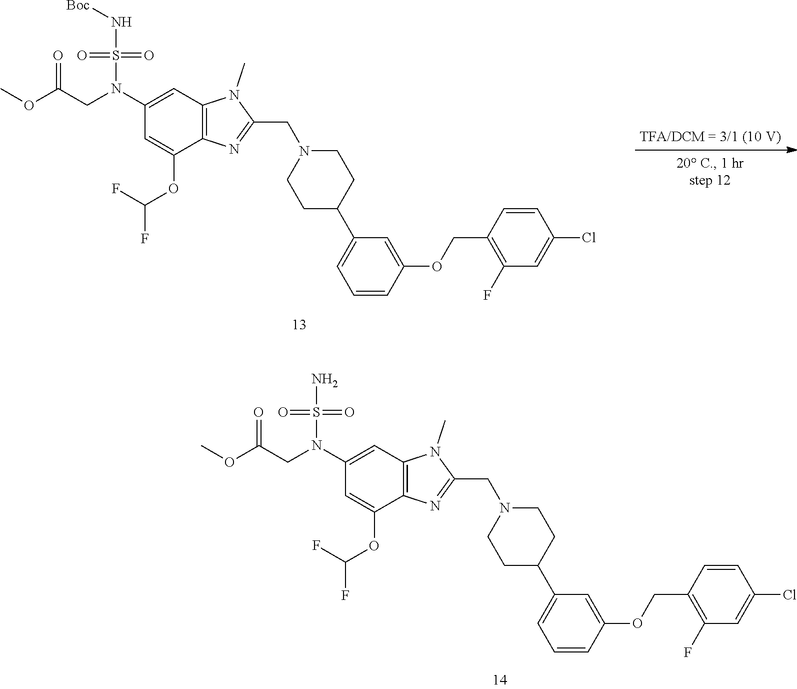 [SMILES: COC(=O)CN(c1cc(OC(F)F)c2nc(CN3CCC(c4cccc(OCc5ccc(Cl)cc5F)c4)CC3)n(C)c2c1)S(=O)(=O)NC(=O)OC(C)(C)C ; COC(=O)CN(c1cc(OC(F)F)c2nc(CN3CCC(c4cccc(OCc5ccc(Cl)cc5F)c4)CC3)n(C)c2c1)S(N)(=O)=O]

To a solution of 13 (200 mg, 251.18 μmol, 1 eq) in DCM (3 mL) was added TFA (1 mL). The mixture was stirred at 20° C. for 1 hr. LC-MS showed 13 was consumed completely and one main peak with desired mass was detected. The mixture was added a.q. Na₂CO₃ 15 mL, and diluted with H₂O (15 mL), extracted with DCM (15 mL*3). The combined organic layer was washed with 10 mL brine, dried over Na₂SO₄, filtered and the filtrate was concentrated to give a crude product. 14 (320 mg, 459.68 μmol, 91% yield) was obtained as a brown solid.

LCMS: RT=1.560 min, MS cal.: 695.2, 696.2, [M+H]⁺=696.1, 697.1

### General Procedure for Preparation of Scheme 2, Compound 281 (15):

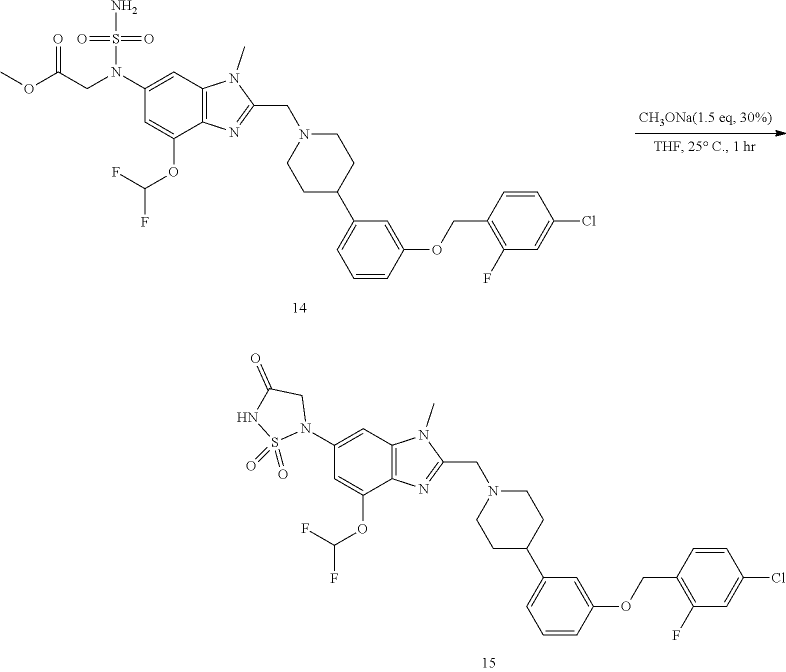 [SMILES: COC(=O)CN(c1cc(OC(F)F)c2nc(CN3CCC(c4cccc(OCc5ccc(Cl)cc5F)c4)CC3)n(C)c2c1)S(N)(=O)=O ; Cn1c(CN2CCC(c3cccc(OCc4ccc(Cl)cc4F)c3)CC2)nc2c(OC(F)F)cc(N3CC(=O)NS3(=O)=O)cc21]

To a solution of 14 (100 mg, 143.65 μmol, 1 eq) in THF (1 mL) was added CH₃ONa (25.87 mg, 143.65 μmol, 30% purity, 1 eq). The mixture was stirred at 25° C. for 1 h. LC-MS showed 14 was consumed completely and desired mass was detected. The reaction mixture was purified by prep-HPLC (neutral condition; column: Waters Xbridge Prep OBD C18 150*40 mm*10 um; mobile phase: [H₂O (10 mM NH₄HCO₃)-ACN]; gradient: 45%-75% B over 8.0 min). 5-(2-((4-(3-((4-Chloro-2-fluorobenzyl)oxy)phenyl)piperidin-1-yl)methyl)-4-(difluoromethoxy)-1-methyl-1H-benzo[d]imidazol-6-yl)-1,2,5-thiadiazolidin-3-one 1,1-dioxide (18.98 mg, 28.15 μmol, 19.60% yield, 98.51% purity) was obtained as a white solid.

LCMS: RT=3.016 min, MS cal.: 663.1, [M+H]⁺=664.2

HPLC: RT=12.150 min, purity: 98.51%

¹H NMR (400 MHz, DMSO-d6) δ=7.81 (s, 1H), 7.62 (s, 1H), 7.57 (t, J=8.0 Hz, 1H), 7.44 (s, 1H), 7.36 (dd, J=2.0, 10.0 Hz, 1H), 7.30 (d, J=8.0 Hz, 1H), 7.26 (t, J=7.6 Hz, 1H), 7.06 (s, 2H), 6.94-6.87 (m, 3H), 5.15 (s, 2H), 4.39 (s, 2H), 4.05 (s, 2H), 3.85 (s, 3H), 3.54-3.42 (m, 2H), 3.01-2.87 (m, 2H), 2.77 (t, J=10.8 Hz, 1H), 2.05-1.86 (m, 4H)

### Example A236—Preparation of Compound 279. (S)-2-((4-(6-((4-Chloro-2-fluorobenzyl)oxy)pyridin-2-yl)piperidin-1-yl)methyl)-4-methoxy-1-(oxetan-2-ylmethyl)-6-(1H-tetrazol-5-yl)-1H-benzo[d]imidazole (Compound 279) (9)

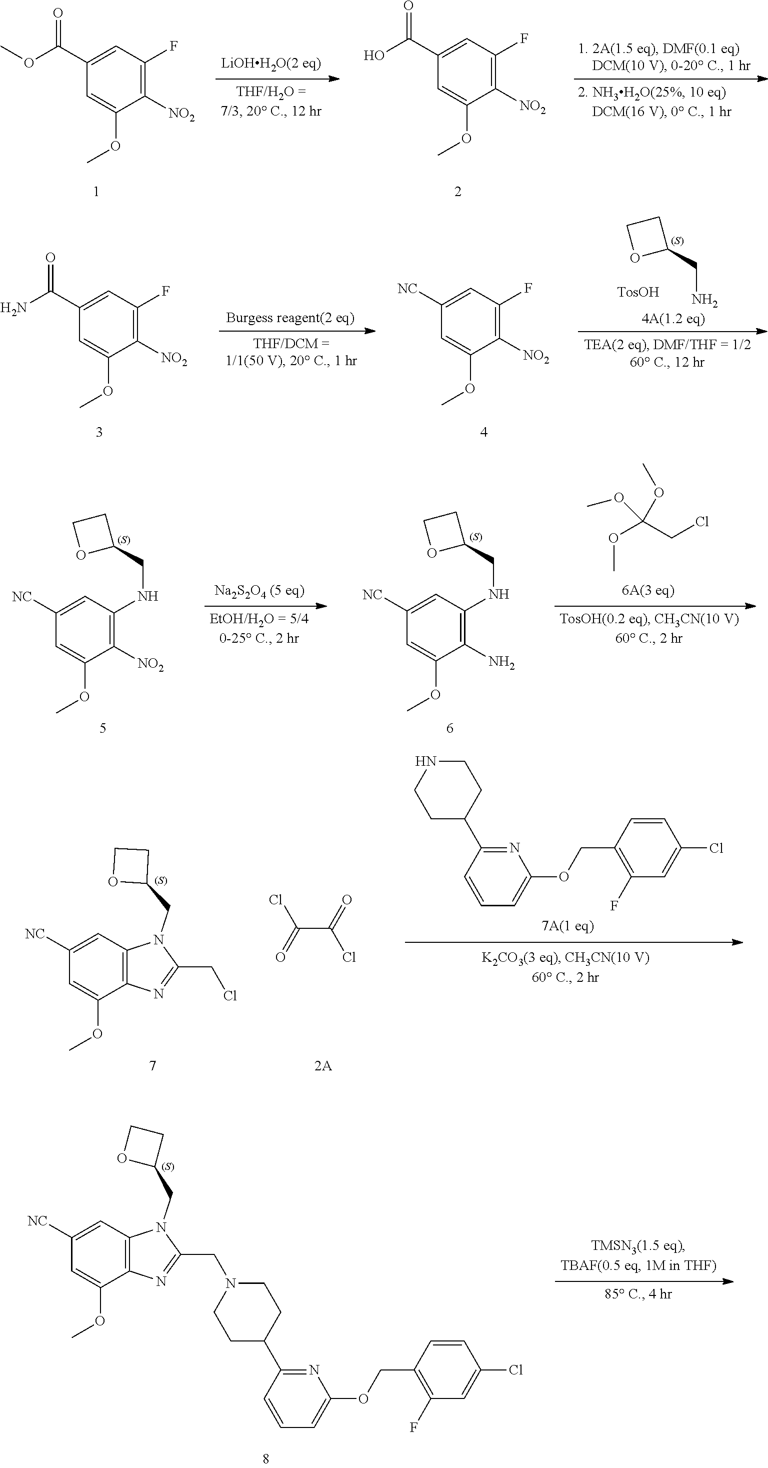

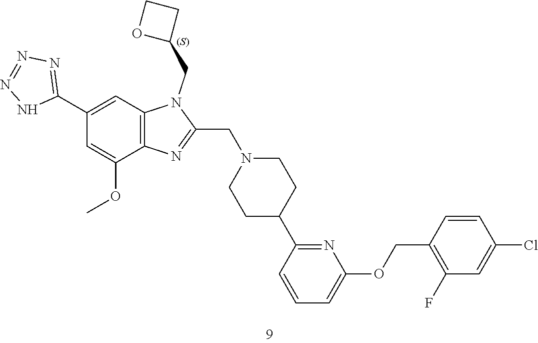

### General Procedure for Preparation of Intermediate 2:

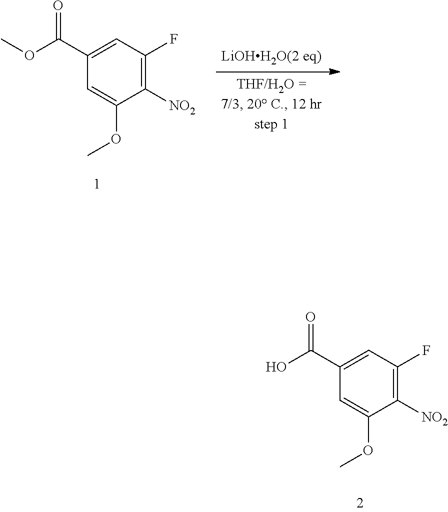 [SMILES: COC(=O)c1cc(F)c([N+](=O)[O-])c(OC)c1 ; COc1cc(C(=O)O)cc(F)c1[N+](=O)[O-]]

To a solution of 1 (1 g, 4.36 mmol, 1 eq) in THF (7 mL) was added LiOH H₂O (366.24 mg, 8.73 mmol, 2 eq) in H₂O (3 mL). The mixture was stirred at 20° C. for 12 hr. TLC (Petroleum ether/Ethyl acetate=1/1, product R_{f}=0.10) indicated 1 was consumed completely and one new spot formed. The reaction was clean according to TLC. HCl (2 M) was added to the mixture to adjust pH=2. The mixture was diluted with H₂O 10 mL and extracted with EtOAc 60 mL (20 mL*3). The combined organic layers were washed with brine 20 mL, dried over Na₂SO₄, filtered, and concentrated under reduced pressure to give the product. 2 (900 mg, 4.18 mmol, 96% yield) was obtained as a white solid.

LCMS: RT=0.707 min, MS cal.: 215.0, [M−H]⁺=213.9

¹H NMR (400 MHz, DMSO-d6) δ=7.60 (s, 1H), 7.50 (d, J=10.0 Hz, 1H), 3.98 (s, 3H)

### General Procedure for Preparation of Intermediate 3:

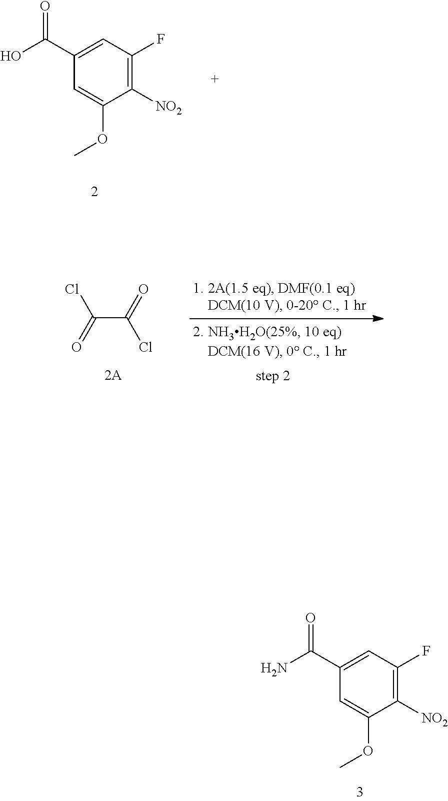

To a solution of 2 (800.00 mg, 3.72 mmol, 1 eq) in DCM (8 mL) was added DMF (27.18 mg, 371.86 μmol, 28.61 μL, 0.1 eq) at 20° C. The mixture was cooled to 0° C. and 2A (708.00 mg, 5.58 mmol, 488.28 μL, 1.5 eq) was added dropwise. The mixture was stirred at 0° C. for 1 hr. TLC (Petroleum ether/Ethyl acetate=0/1, R_{f}=0.67) indicated 2 was consumed completely and one new spot formed. The reaction was clean according to TLC. The mixture was concentrated under reduced pressure to remove solvent. The residue was diluted with DCM (8 mL) to form solution A. To a solution of NH₃·H₂O (5.21 g, 37.19 mmol, 5.73 mL, 25% purity, 10 eq) in DCM (5 mL) was added solution A at 0° C. The mixture was stirred at 0° C. for another 1 hr. TLC (Petroleum ether/Ethyl acetate=0/1, R_{f}=0.67) indicated 2 was consumed completely and one new spot formed. The reaction was clean according to TLC. The reaction mixture was diluted with H₂O 10 mL and extracted with DCM 30 mL (10 mL*3). The combined organic layers were washed with brine 10 mL, dried over Na₂SO₄, filtered and concentrated under reduced pressure to give a residue. The residue was purified by column chromatography (SiO₂, Petroleum ether/Ethyl acetate=10/1 to 2/1). 3 (600 mg, 2.80 mmol, 75% yield) was obtained as a white solid.

LCMS: RT=0.918 min, MS cal.: 214.0, [M−H]⁻=213.0

¹H NMR (400 MHz, DMSO-d6) δ=8.26 (s, 1H), 7.83 (s, 1H), 7.63 (s, 1H), 7.57 (dd, J=1.2, 10.4 Hz, 1H), 4.00 (s, 3H)

### General Procedure for Preparation of Intermediate 4:

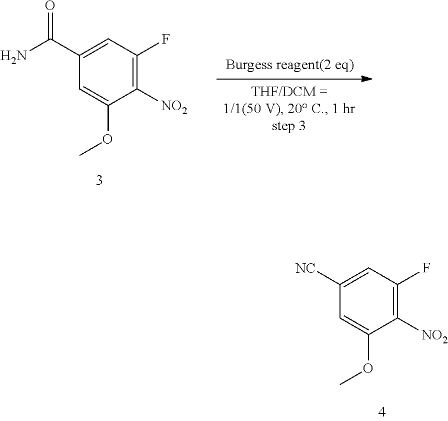

To a solution of 3 (290 mg, 1.35 mmol, 1 eq) in THF (1.5 mL) was added Burgess reagent (645.42 mg, 2.71 mmol, 2 eq) in DCM (1.5 mL). The mixture was stirred at 20° C. for 1 hr. TLC (Petroleum ether/Ethyl acetate=0/1, R_{f}=0.60) indicated 3 was consumed completely and one new spot formed. The reaction was clean according to TLC. The reaction mixture was diluted with H₂O 10 mL and extracted with DCM 30 mL (10 mL*3). The combined organic layers were washed with brine 10 mL, dried over Na₂SO₄, filtered, and concentrated under reduced pressure to give a residue. The residue was purified by column chromatography (SiO₂, Petroleum ether/Ethyl acetate=10/1 to 5/1). 4 (430 mg, 2.19 mmol, 78% yield) was obtained as a white solid.

LCMS: RT=1.114 min, MS cal.: 196.0, [M−H]⁺=195.0

¹H NMR (400 MHz, DMSO-d6) δ=7.90 (dd, J=1.2, 9.6 Hz, 1H), 7.88 (d, J=1.2 Hz, 1H), 4.01 (s, 3H)

### General Procedure for Preparation of Intermediate 5:

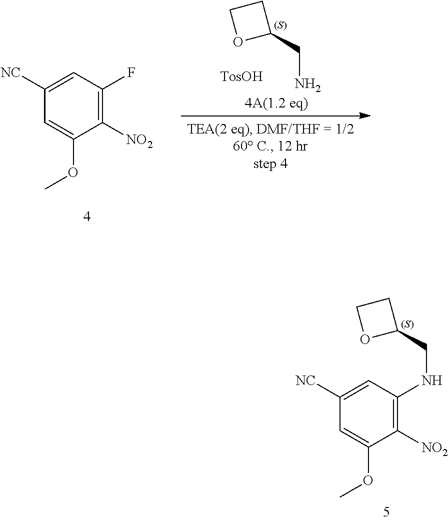

To a solution of 4 (330 mg, 1.68 mmol, 1 eq) in DMF (1 mL) and THF (2 mL) was added 4A (523.57 mg, 2.02 mmol, 1.2 eq, p-TSA) and TEA (340.51 mg, 3.37 mmol, 468.37 μL, 2 eq). The mixture was stirred at 60° C. for 12 hr. TLC (Petroleum ether/Ethyl acetate=3/1, product R_{f}=0.30) indicated 4 was consumed completely and one new spot formed. The reaction was clean according to TLC. The reaction mixture was diluted with H₂O 20 mL and extracted with EtOAc 60 mL (20 mL*3). The combined organic layers were washed with brine 20 mL, dried over Na₂SO₄, filtered and concentrated under reduced pressure to give a residue. The residue was purified by column chromatography (SiO₂, Petroleum ether/Ethyl acetate=10/1 to 3/1). 5 (380 mg, 1.44 mmol, 86% yield) was obtained as a yellow oil.

LCMS: RT=1.086 min, MS cal.: 263.1, [M+H]⁺=263.9

¹H NMR (400 MHz, DMSO-d6) δ=7.12 (s, 1H), 6.90 (s, 1H), 6.54 (t, J=5.6 Hz, 1H), 4.87-4.80 (m, 1H), 4.51-4.44 (m, 1H), 4.41-4.33 (m, 1H), 3.85 (s, 3H), 3.50-3.38 (m, 2H), 2.68-2.54 (m, 1H), 2.45-2.31 (m, 1H)

### General Procedure for Preparation of Intermediate 6:

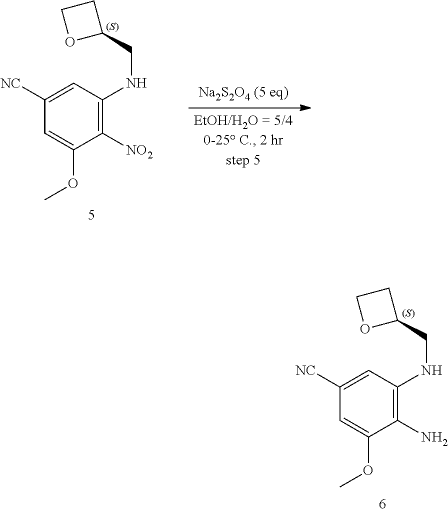

To a solution of 5 (380 mg, 1.44 mmol, 1 eq) in EtOH (3 mL) was added Na₂S₂O₄ (1.26 g, 7.22 mmol, 1.57 mL, 5 eq) in H₂O (2.4 mL) at 0° C. The mixture was stirred at 25° C. for 2 hr. TLC (Petroleum ether/Ethyl acetate=1/1, R_{f}=0.20) indicated 5 was consumed completely and one new spot formed. The reaction was clean according to TLC. The reaction mixture was diluted with H₂O 10 mL and extracted with EtOAc 30 mL (10 mL*3). The combined organic layers were washed with brine 10 mL, dried over Na₂SO₄, filtered, and concentrated under reduced pressure to give the product. Compound 6 (180 mg, 771.65 μmol, 53% yield) was obtained as a yellow oil.

LCMS: RT=0.924 min, MS cal.: 233.1, [M+H]⁺=234.0

¹H NMR (400 MHz, DMSO-d6) δ=6.73 (d, J=1.2 Hz, 1H), 6.59 (d, J=1.2 Hz, 1H), 5.13 (s, 2H), 5.02 (t, J=5.6 Hz, 1H), 4.92-4.84 (m, 1H), 4.55-4.49 (m, 1H), 4.47-4.41 (m, 1H), 3.77 (s, 3H), 3.41-3.35 (m, 1H), 3.32-3.24 (m, 1H), 2.69-2.58 (m, 1H), 2.46-2.36 (m, 1H)

### General Procedure for Preparation of Intermediate 7:

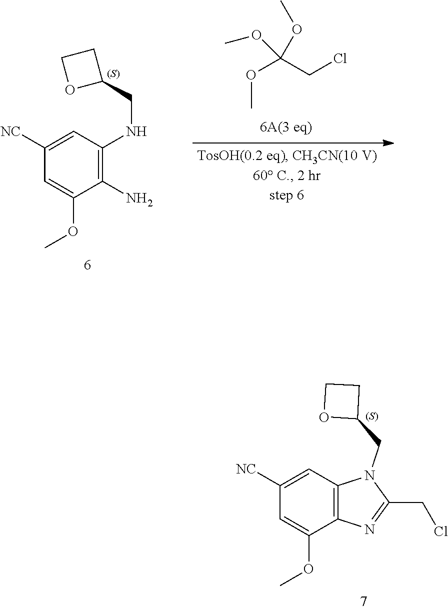

To a solution of 6 (180 mg, 771.65 μmol, 1 eq) in ACN (2 mL) was added TosOH (26.58 mg, 154.33 μmol, 0.2 eq) and 6A (357.87 mg, 2.31 mmol, 312.01 μL, 3 eq). The mixture was stirred at 60° C. for 2 hr. LC-MS (RT=0.978) showed 6 was consumed completely and desired mass was detected. The reaction mixture was diluted with H₂O 10 mL and extracted with DCM 30 mL (10 mL*3). The combined organic layers were washed with brine 10 mL, dried over Na₂SO₄, filtered, and concentrated under reduced pressure to give a residue. The crude product was triturated with MTBE 2 mL at 20° C. for 10 min. The mixture was filtered, and the filter cake was concentrated under reduced pressure to give a residue. 7 (175 mg, 599.87 μmol, 77.74% yield) was obtained as a yellow solid.

LCMS: RT=0.978 min, MS cal.: 291.1, 293.1, [M+H]⁺=291.9, 293.9

¹H NMR (400 MHz, DMSO-d6) δ=7.93 (s, 1H), 7.15 (s, 1H), 5.15-5.07 (m, 2H), 5.07-5.00 (m, 1H), 4.75-4.66 (m, 1H), 4.61-4.53 (m, 1H), 4.50-4.42 (m, 1H), 4.37-4.31 (m, 1H), 3.98 (s, 3H), 2.73-2.62 (m, 1H), 2.40-2.27 (m, 1H)

### General Procedure for Preparation of Intermediate 8:

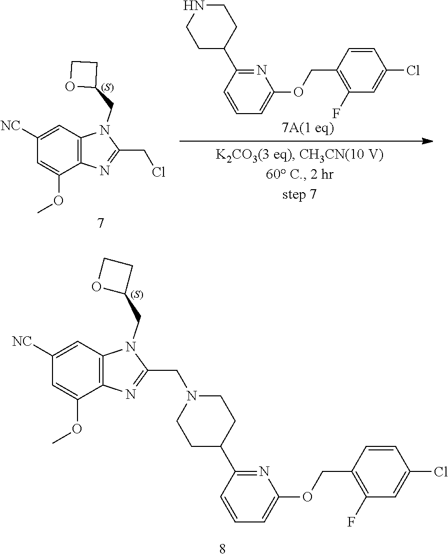

To a solution of 7 (175 mg, 599.87 μmol, 1 eq) in ACN (2 mL) was added K₂CO₃ (248.71 mg, 1.80 mmol, 3 eq) and 7A (192.43 mg, 599.87 μmol, 1 eq). The mixture was stirred at 60° C. for 2 hr. TLC (Petroleum ether/Ethyl acetate=1/1, R_{f}=0.15) indicated 7 was consumed completely and one new spot formed. The reaction was clean according to TLC. The reaction mixture was diluted with H₂O 10 mL and extracted with EtOAc 30 mL (10 mL*3). The combined organic layers were washed with brine 10 mL, dried over Na₂SO₄, filtered, and concentrated under reduced pressure to give a residue. The residue was purified by column chromatography (SiO₂, Petroleum ether/Ethyl acetate=10/1 to 1/3). 8 (190 mg, 329.83 μmol, 54.98% yield) was obtained as a white solid.

LCMS: RT=1.605 min, MS cal.: 575.2, 576.2, [M+H]⁺=576.1, 577.1

¹H NMR (400 MHz, DMSO-d6) δ=7.86 (s, 1H), 7.62 (t, J=7.6 Hz, 1H), 7.56 (t, J=8.0 Hz, 1H), 7.46 (dd, J=2.0, 10.4 Hz, 1H), 7.29 (dd, J=1.6, 8.4 Hz, 1H), 7.10 (s, 1H), 6.87 (d, J=7.2 Hz, 1H), 6.67 (d, J=8.0 Hz, 1H), 5.37 (s, 2H), 5.10 (dq, J=2.4, 7.2 Hz, 1H), 4.82-4.74 (m, 1H), 4.67-4.58 (m, 1H), 4.50-4.43 (m, 1H), 4.42-4.37 (m, 1H), 3.96 (s, 3H), 3.93 (d, J=13.6 Hz, 1H), 3.77 (d, J=13.6 Hz, 1H), 2.97 (d, J=11.2 Hz, 1H), 2.82 (d, J=11.2 Hz, 1H), 2.74-2.64 (m, 1H), 2.62-2.55 (m, 1H), 2.45-2.35 (m, 1H), 2.27-2.11 (m, 2H), 1.84-1.60 (m, 4H)

### General Procedure for Preparation of Compound 279 (9):

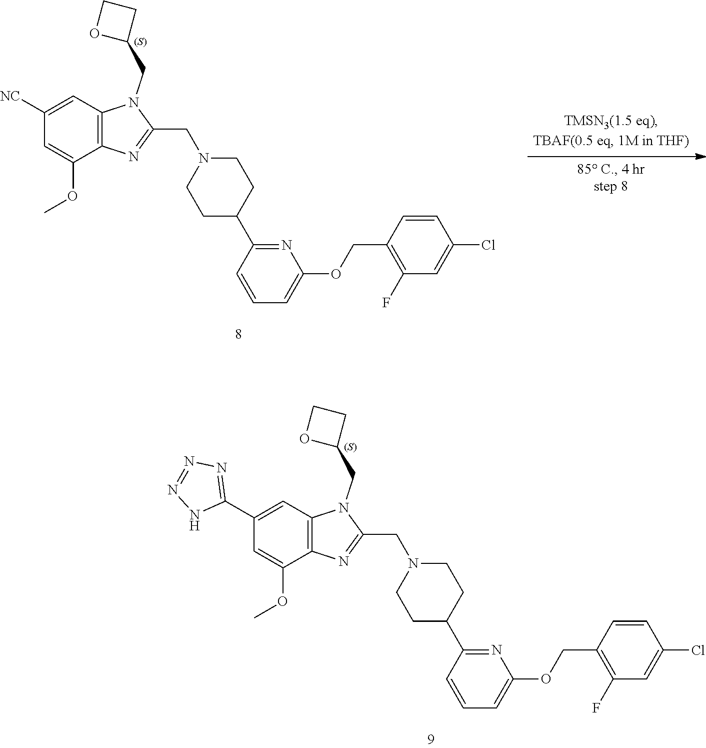

Equip a 10 mL round bottom flask, addition funnel and N₂ balloon. 8 (88 mg, 152.76 μmol, 1 eq) was charged to the round bottom flask, then TBAF (1 M, 76.38 μL, 0.5 eq) was added to the mixture at 20° C. At 20° C., TMSN₃ (26.40 mg, 229.14 μmol, 30.14 μL, 1.5 eq) was added to the reaction mixture. After the addition, the mixture was stirred at 85° C. for 4 hr. Then TMSN₃ (26.40 mg, 229.14 μmol, 30.14 μL, 1.5 eq) and TBAF (1 M, 76.38 μL, 0.5 eq) was added to the mixture and stirred at 85° C. for 4 hr. Then TMSN₃ (26.40 mg, 229.14 μmol, 30.14 μL, 1.5 eq) and TBAF (1 M, 76.38 μL, 0.5 eq) was added to the mixture and stirred at 85° C. for 12 hr. LCMS (RT=0.997 min) showed desired compound was detected. The reaction mixture was cooled to room temperature, 0.5 mL THF and 2 mL MeCN was added to the suspension, then the reaction mixture became clear. The reaction mixture was purified by prep-HPLC (column: Phenomenex Luna C18 100*30 mm*3 um; mobile phase: [H₂O (0.2% FA)-ACN]; gradient: 15%-45% B over 8.0 min). (S)-2-((4-(6-((4-Chloro-2-fluorobenzyl)oxy)pyridin-2-yl)piperidin-1-yl)methyl)-4-methoxy-1-(oxetan-2-ylmethyl)-6-(1H-tetrazol-5-yl)-1H-benzo[d]imidazole (12.04 mg, 19.45 μmol, 12.73% yield) was obtained as a white solid.

LCMS: RT=2.840 min, MS cal.: 618.2, [M+H]⁺=619.3

HPLC: RT=11.663 min, purity: 97.41%

¹H NMR (400 MHz, METHANOL-d4) δ=7.85 (s, 1H), 7.62 (t, J=8.0 Hz, 1H), 7.57 (s, 1H), 7.50 (t, J=8.4 Hz, 1H), 7.26-7.17 (m, 2H), 6.89 (d, J=7.2 Hz, 1H), 6.69 (d, J=8.4 Hz, 1H), 5.43 (s, 2H), 5.35-5.26 (m, 1H), 4.83-4.76 (m, 1H), 4.73-4.64 (m, 2H), 4.52 (s, 2H), 4.49-4.44 (m, 1H), 4.11 (s, 3H), 3.66-3.53 (m, 2H), 3.10-2.99 (m, 2H), 2.99-2.90 (m, 1H), 2.87-2.77 (m, 1H), 2.64-2.52 (m, 1H), 2.13-2.02 (m, 4H)

### Example A237—Preparation of Compound 278. (R)-2-((4-(2-(4-Cyano-2-fluorophenyl)-2,3-dihydrobenzo[b][1,4]dioxin-5-yl)piperidin-1-yl)methyl)-4-methoxy-1-methyl-1H-benzo[d]imidazole-6-carboxylic acid (Compound 278) (3)

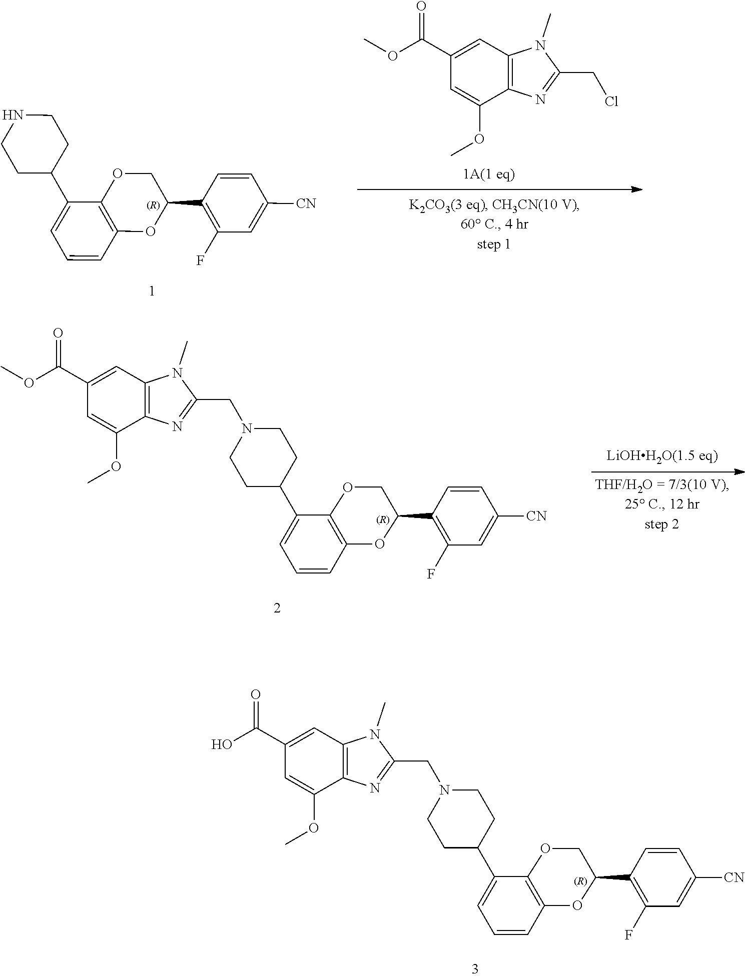

### General Procedure for Preparation of Intermediate 2:

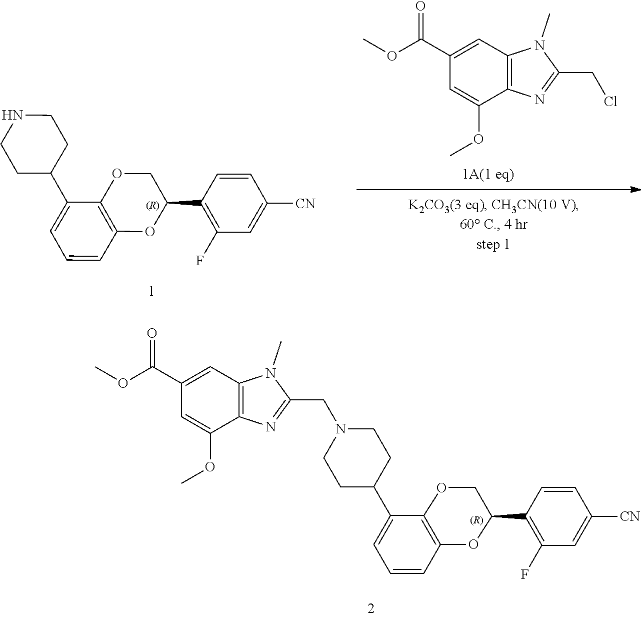

To a solution of 1 (100 mg, 221.04 μmol, 1 eq, TFA), 1A (59.39 mg, 221.04 μmol, 1 eq) in CH₃CN (1 mL) was added K₂CO₃ (91.65 mg, 663.13 μmol, 3 eq). The mixture was stirred at 60° C. for 4 hr. LC-MS showed 1 was consumed completely and desired mass was detected. The residue was diluted with H₂O 20 mL and extracted with EtOAc (10 mL*3). The combined organic layers were washed with brine 20 mL, dried over Na₂SO₄ filtered and concentrated under reduced pressure to give residue. The residue was purified by column chromatography (SiO₂, Petroleum ether/Ethyl acetate=1/1 to 0/1). 2 (110 mg, 192.78 μmol, 87.21% yield) was obtained as a white solid.

LCMS: RT=1.499 min, MS cal.: 570.62, [M+H]⁺=571.2

¹H NMR (400 MHz, CHLOROFORM-d) δ=7.78 (d, J=1.2 Hz, 1H), 7.68 (t, J=7.6 Hz, 1H), 7.54 (dd, J=1.2, 8.0 Hz, 1H), 7.43 (dd, J=1.2, 9.6 Hz, 1H), 7.39 (s, 1H), 6.91-6.80 (m, 3H), 5.48 (dd, J=2.0, 7.6 Hz, 1H), 4.51 (dd, J=2.4, 11.2 Hz, 1H), 4.09-4.03 (m, 3H), 3.97 (s, 7H), 3.88 (d, J=3.2 Hz, 2H), 2.96 (s, 3H), 2.38-2.24 (m, 2H), 1.85 (d, J=12.0 Hz, 1H), 1.71-1.55 (m, 2H)

### General Procedure for Preparation of Compound 278 (3):

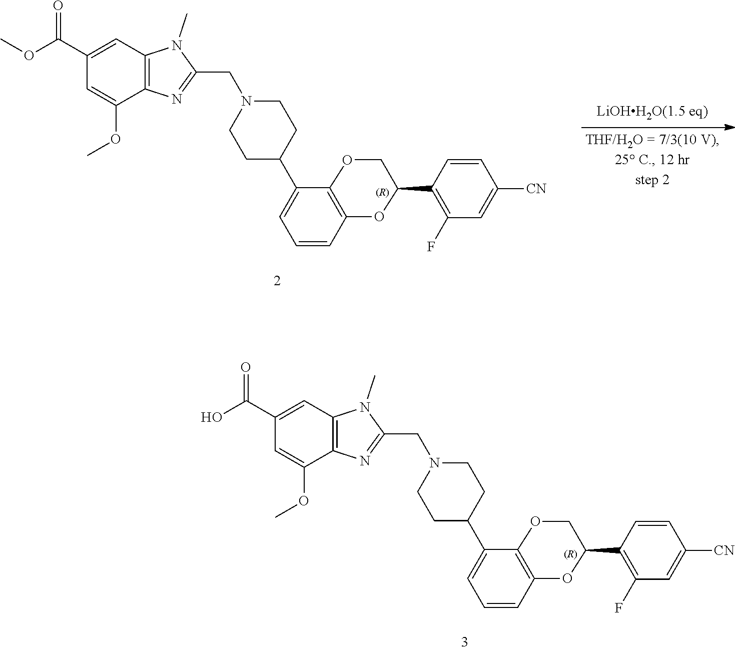

To a solution of 2 (110 mg, 192.78 μmol, 1 eq) in THF (0.7 mL) was added LiOH H₂O (12.13 mg, 289.16 μmol, 1.5 eq) in H₂O (0.3 mL). The mixture was stirred at 25° C. for 12 hr. LC-MS showed 2 was consumed completely and desired mass was detected. The reaction mixture was purified directly. The reaction was purified by prep-HPLC (neutral condition; column: Waters Xbridge Prep OBD C18 150*40 mm*10 um; mobile phase: [H₂O (10 mM NH₄HCO₃)-ACN]; gradient: 25%-65% B over 8.0 min). (R)-2-((4-(2-(4-Cyano-2-fluorophenyl)-2,3-dihydrobenzo[b][1,4]dioxin-5-yl)piperidin-1-yl)methyl)-4-methoxy-1-methyl-1H-benzo[d]imidazole-6-carboxylic acid (29.65 mg, 53.27 μmol, 27.63% yield) was obtained as a white solid.

LCMS: RT=2.594 min, MS cal.: 556.59, [M+H]⁺=557.2

HPLC: RT=10.317 min, purity: 99.50%

¹H NMR (400 MHz, METHANOL-d₄) δ=7.83 (s, 1H), 7.74-7.69 (m, 1H), 7.68-7.61 (m, 2H), 7.45 (s, 1H), 6.86-6.81 (m, 3H), 5.53-5.48 (m, 1H), 4.52 (dd, J=2.4, 11.2 Hz, 1H), 4.09-4.04 (m, 1H), 4.02 (s, 3H), 3.97 (s, 3H), 3.88 (s, 2H), 3.03 (d, J=9.2 Hz, 2H), 2.99-2.89 (m, 1H), 2.37-2.26 (m, 2H), 1.89-1.71 (m, 4H)

### Example A238—Preparation of Compound 277. (S)-2-((4-(2-(4-Cyano-2-fluorophenyl)-2,3-dihydrobenzo[b][1,4]dioxin-5-yl)piperidin-1-yl)methyl)-4-ethoxy-1-methyl-1H-benzo[d]imidazole-6-carboxylic acid (Compound 277) (3)

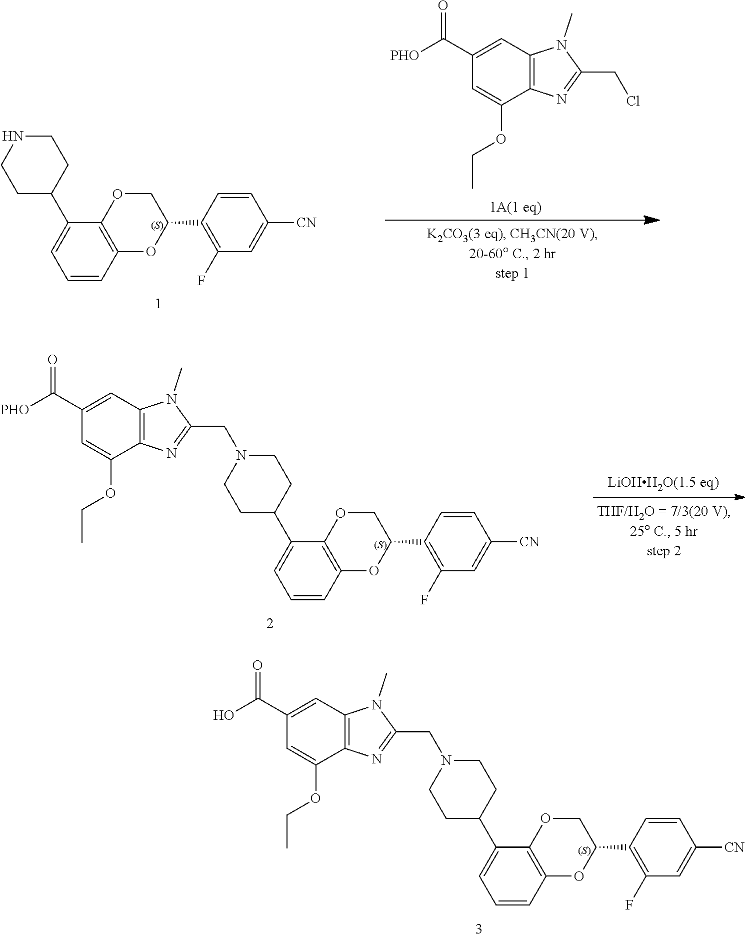

### General Procedure for Preparation of Intermediate 2:

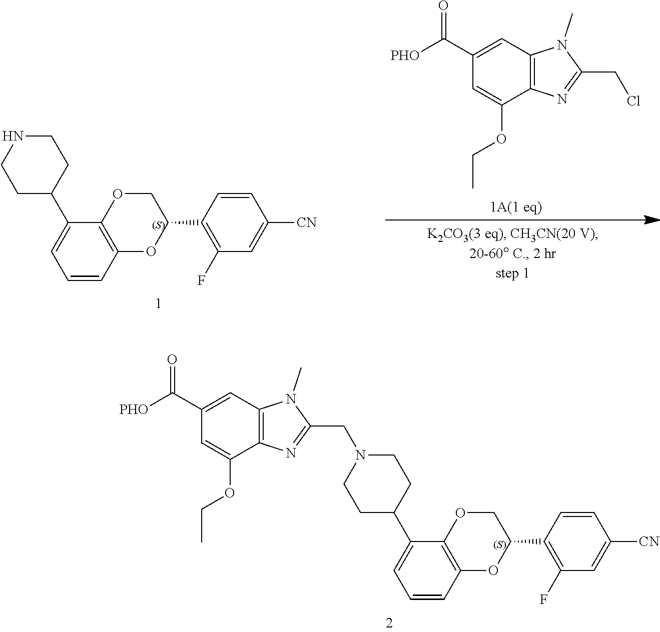

To a solution of 1 (50.00 mg, 147.77 μmol, 1 eq) in CH₃CN (1 mL) was added K₂CO₃ (61.27 mg, 443.30 μmol, 3 eq) and 1A (50.95 mg, 147.77 μmol, 1 eq) at 20° C. The mixture was stirred at 60° C. for 2 hr. TLC (PE/EtOAc=1/1, product Rf=0.30) showed the starting material was consumed completely. The reaction mixture was cooled down to 20° C., then added H₂O 3 mL, and extracted with EtOAc (5 mL*3). The combined organic layers were washed with brine (3 mL*2), dried over Na₂SO₄, filtered, and concentrated under reduced pressure to give a residue. The residue was purified by column chromatography (SiO₂, PE/EtOAc=10/1 to 0/1). 2 (80 mg, 123.70 μmol, 83.72% yield) was obtained as a yellow solid.

¹H NMR (400 MHz, CHLOROFORM-d) δ=7.93 (s, 1H), 7.69 (t, J=7.6 Hz, 1H), 7.58-7.51 (m, 2H), 7.50-7.40 (m, 3H), 7.34-7.28 (m, 1H), 7.25 (s, 2H), 6.92-6.81 (m, 3H), 5.49 (d, J=6.4 Hz, 1H), 5.31 (s, 1H), 4.52 (dd, J=2.0, 11.2 Hz, 1H), 4.37 (q, J=7.2 Hz, 2H), 4.07-3.88 (m, 6H), 2.97 (s, 3H), 2.33 (d, J=1.6 Hz, 2H), 1.90-1.65 (m, 4H)

### General Procedure for Preparation of Compound 277 (3):

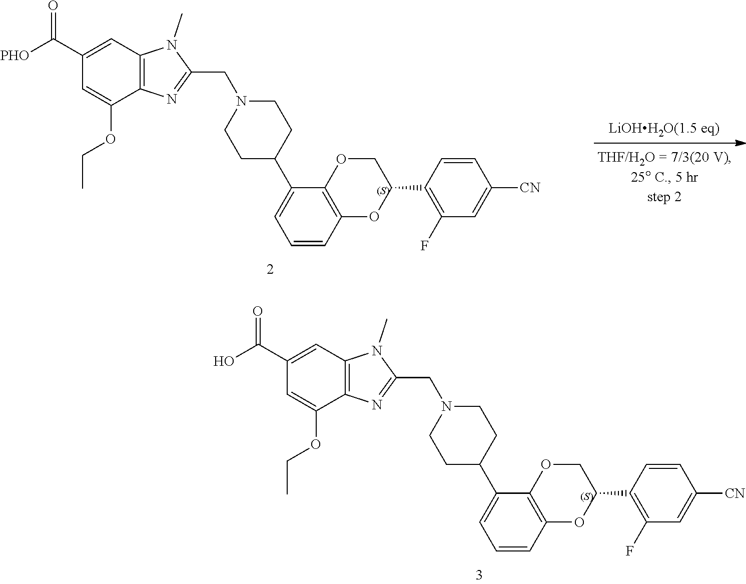

To a solution of 2 (70 mg, 108.24 μmol, 1 eq) in THF (0.98 mL) was added LiOH·H₂O (6.81 mg, 162.36 μmol, 1.5 eq) in H₂O (0.42 mL). The mixture was stirred at 25° C. for 5 hr. LCMS (RT=0.435 min) showed the starting material was consumed completely. The reaction mixture was filtered. The filtrate was purified by prep-HPLC (column: Waters Xbridge Prep OBD C18 150*40 mm*10 um; mobile phase: [H₂O (10 mM NH₄HCO₃)—CH₃CN]; gradient: 15%-65% B over 8.0 min). (S)-2-((4-(2-(4-Cyano-2-fluorophenyl)-2,3-dihydrobenzo[b][1,4]dioxin-5-yl)piperidin-1-yl)methyl)-4-ethoxy-1-methyl-1H-benzo[d]imidazole-6-carboxylic acid (18.09 mg, 31.10 μmol, 28.73% yield, 98.09% purity) was obtained as a white solid.

LCMS: RT=2.638 min, MS cal.: 570.2, [M+H]⁺=571.3

HPLC: RT=10.809 min

¹H NMR (400 MHz, METHANOL-d4) δ=7.82 (d, J=1.2 Hz, 1H), 7.74-7.68 (m, 1H), 7.67-7.61 (m, 2H), 7.42 (s, 1H), 6.88-6.79 (m, 3H), 5.54-5.46 (m, 1H), 4.52 (dd, J=2.0, 11.4 Hz, 1H), 4.29 (q, J=7.2 Hz, 2H), 4.06 (dd, J=7.6, 11.6 Hz, 1H), 3.97 (s, 3H), 3.90 (s, 2H), 3.04 (d, J=9.2 Hz, 2H), 3.00-2.89 (m, 1H), 2.41-2.27 (m, 2H), 1.90-1.69 (m, 4H), 1.52 (t, J=7.2 Hz, 3H)

### Example A239—Preparation of Compound 276. (R)-2-((4-(2-(4-Cyano-2-fluorophenyl)-2,3-dihydrobenzo[b][1,4]dioxin-5-yl)piperidin-1-yl)methyl)-4-ethoxy-1-methyl-1H-benzo[d]imidazole-6-carboxylic acid (Compound 276) (3)

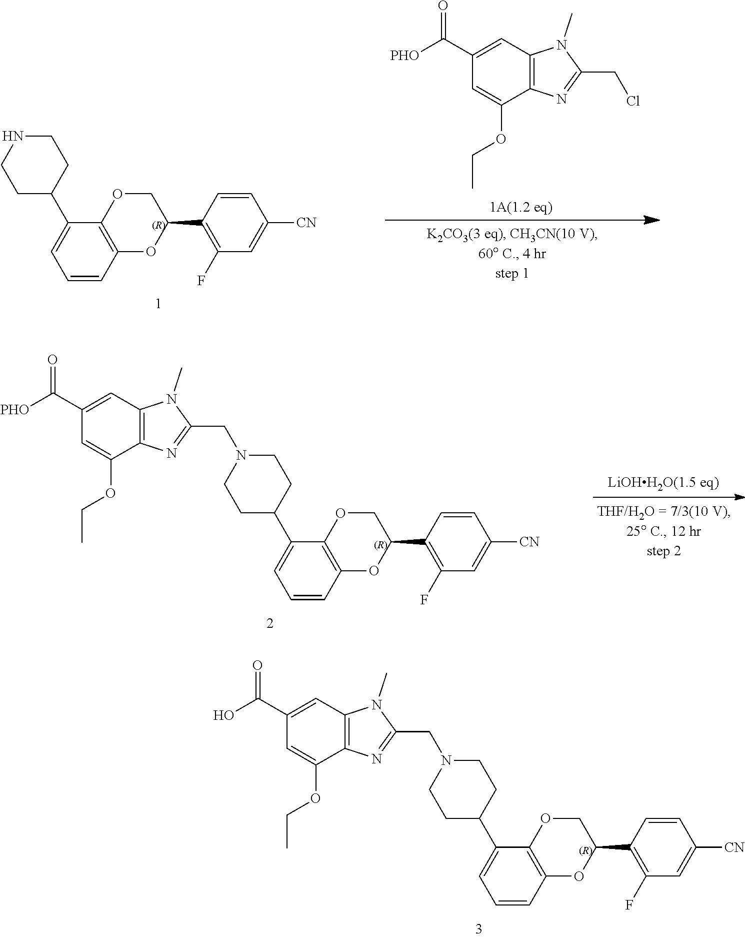

### General Procedure for Preparation of Intermediate 2:

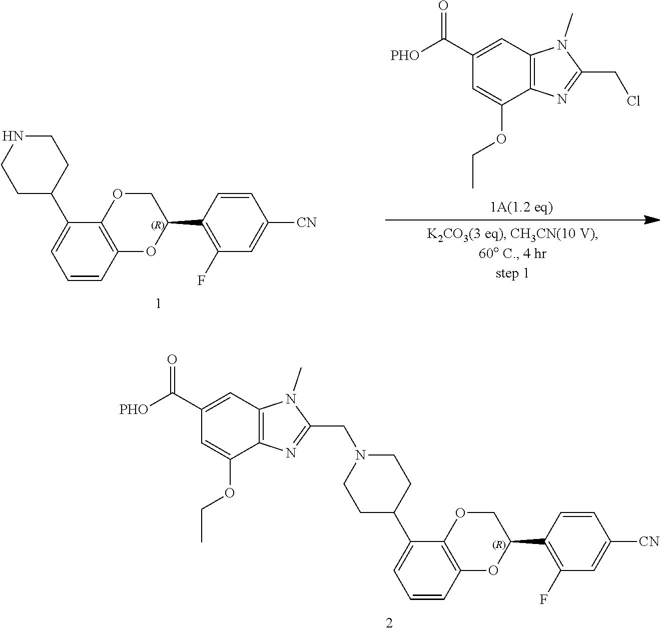

To a solution of 1 (90 mg, 198.94 μmol, 1 eq, TFA), 1A (82.31 mg, 238.73 μmol, 1.2 eq) in CH₃CN (1 mL) was added K₂CO₃ (82.48 mg, 596.82 μmol, 3 eq). The mixture was stirred at 60° C. for 4 hr. LC-MS showed 1 was consumed completely and desired mass was detected. The reaction mixture was diluted with H₂O 20 mL and extracted with EtOAc (10 mL*3). The combined organic layers were washed with brine 20 mL, dried over Na₂SO₄ filtered and concentrated under reduced pressure to give residue. The residue was purified by column chromatography (SiO₂, Petroleum ether/Ethyl acetate=3/1 to 1/1). 2 (60 mg, 92.78 μmol, 46.64% yield) was obtained as a red solid.

LCMS: RT=RT=1.729 min, MS cal.: 646.72, [M+H]⁺=647.2

¹H NMR (400 MHz, CHLOROFORM-d) δ=7.93 (s, 1H), 7.72-7.65 (m, 1H), 7.57-7.51 (m, 2H), 7.51-7.38 (m, 4H), 7.33-7.29 (m, 1H), 7.24 (s, 1H), 6.91-6.82 (m, 3H), 5.49 (d, J=6.4 Hz, 1H), 4.52 (dd, J=2.0, 11.3 Hz, 1H), 4.37 (q, J=7.2 Hz, 2H), 4.13 (q, J=7.2 Hz, 1H), 4.01 (s, 3H), 3.98-3.93 (m, 2H), 3.07-2.86 (m, 3H), 2.33 (dd, J=2.0, 5.6 Hz, 2H), 1.87 (d, J=11.6 Hz, 1H), 1.59 (t, J=7.2 Hz, 4H)

### General Procedure for Preparation of Compound 276 (3):

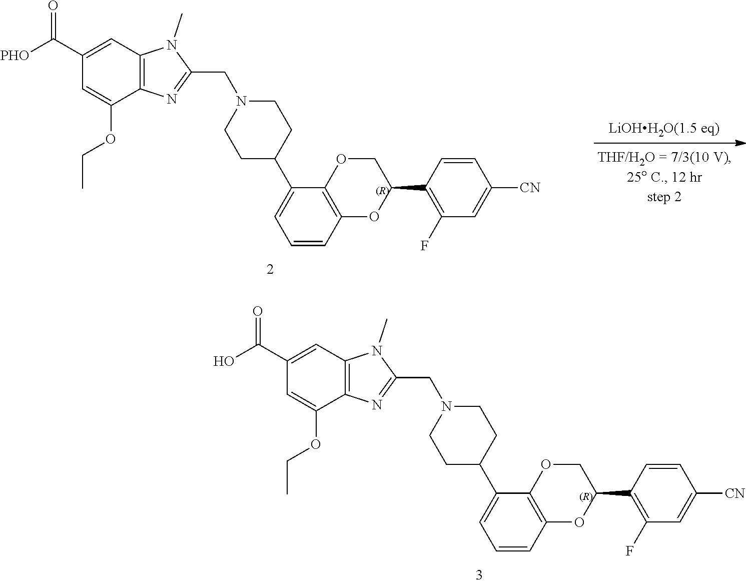

To a solution of 2 (60 mg, 92.78 μmol, 1 eq) in THF (0.7 mL) was added LiOH H₂O (5.84 mg, 139.17 μmol, 1.5 eq) in H₂O (0.3 mL). The mixture was stirred at 25° C. for 12 hr. LC-MS showed 2 was consumed completely and desired mass was detected. The reaction mixture was purified directly. The reaction was purified by prep-HPLC (neutral condition; column: Waters Xbridge Prep OBD C18 150*40 mm*10 um; mobile phase: [H₂O (10 mM NH₄HCO₃)-ACN]; gradient: 25%-55% B over 8.0 min). (R)-2-((4-(2-(4-Cyano-2-fluorophenyl)-2,3-dihydrobenzo[b][1,4]dioxin-5-yl)piperidin-1-yl)methyl)-4-ethoxy-1-methyl-1H-benzo[d]imidazole-6-carboxylic acid (15.26 mg, 26.74 μmol, 28.83% yield) was obtained as a white solid.

LCMS: RT=2.624 min, MS cal.: 570.62, [M+H]⁺=571.1

HPLC: RT=10.736 min, purity: 99.24%

¹H NMR (400 MHz, METHANOL-d₄) δ=7.84 (d, J=1.2 Hz, 1H), 7.74-7.69 (m, 1H), 7.67-7.61 (m, 2H), 7.42 (s, 1H), 6.87-6.80 (m, 3H), 5.50 (dd, J=2.4, 7.6 Hz, 1H), 4.52 (dd, J=2.4, 11.2 Hz, 1H), 4.29 (q, J=6.8 Hz, 2H), 4.06 (dd, J=7.6, 11.2 Hz, 1H), 3.97 (s, 3H), 3.94 (s, 2H), 3.11-3.04 (m, 2H), 3.02-2.91 (m, 1H), 2.43-2.32 (m, 2H), 1.91-1.71 (m, 4H), 1.53 (t, J=7.2 Hz, 3H)

### Example A240—Preparation of Compound 275. 7-((4-(6-((4-Chloro-2-fluorobenzyl)oxy)pyridin-2-yl)piperidin-1-yl)methyl)-6-methyl-6H-[1,3]dioxolo[4′,5′:3,4]benzo[1,2-d]imidazole-4-carboxylic acid (Compound 275) (11)

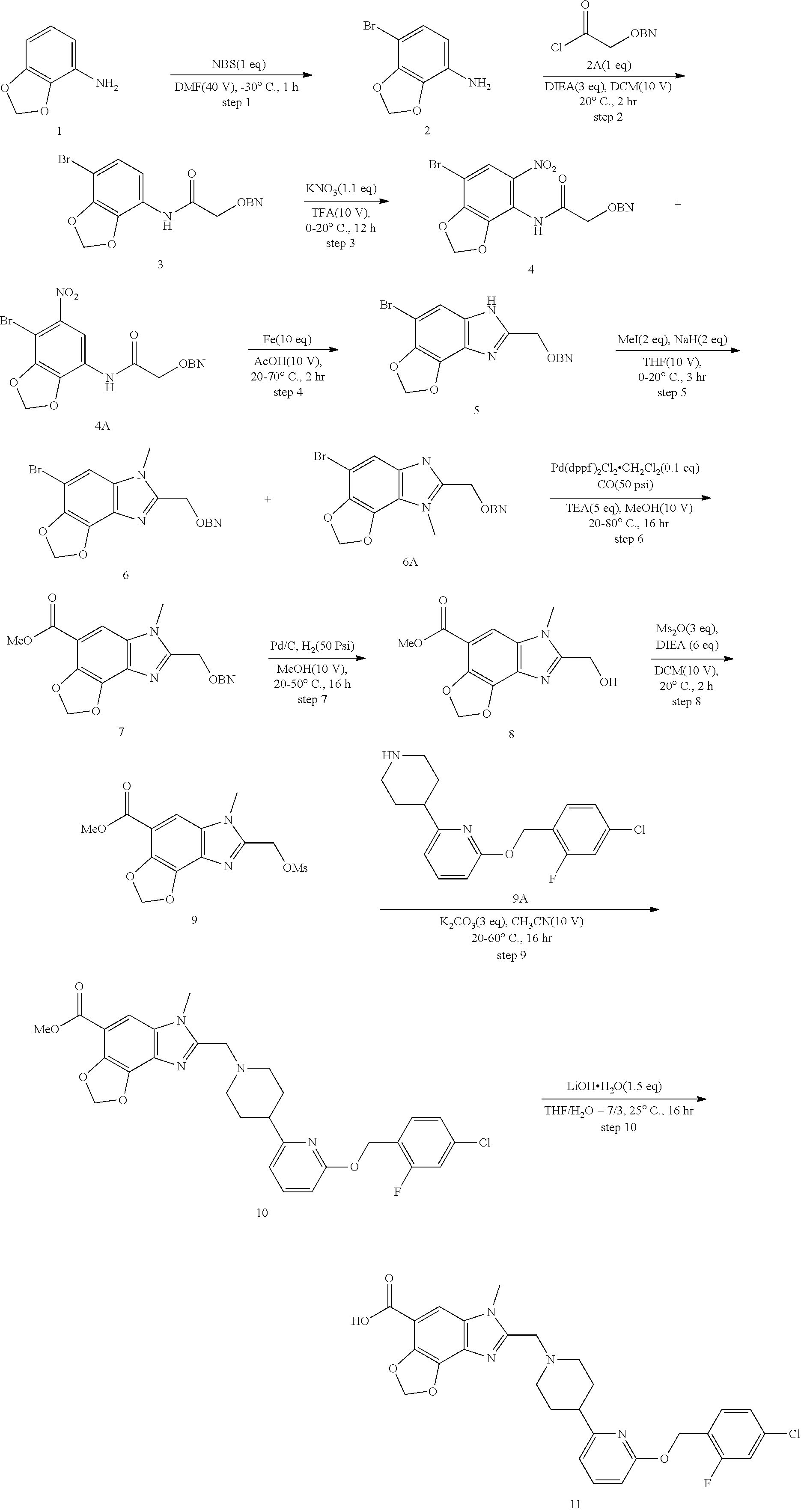

### General Procedure for Preparation of Intermediate 2:

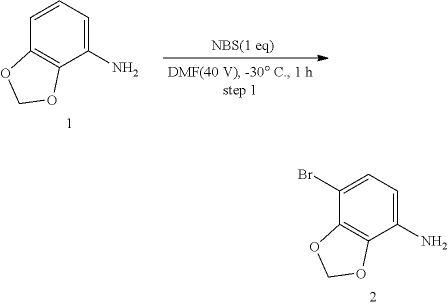 [SMILES: Nc1cccc2c1OCO2 ; Nc1ccc(Br)c2c1OCO2]

A mixture of 1 (10 g, 72.92 mmol, 1 eq) in DMF (200 mL) was degassed and purged with N₂ for 3 times at −30° C., NBS (12.98 g, 72.92 mmol, 1 eq) in DMF (200 mL) was added dropwise to the mixture, then the mixture was stirred at −30° C. for 1 hr under N₂ atmosphere. TLC (Petroleum ether/Ethyl acetate=3/1, R_{f}=0.46) indicated 1 was consumed completely and one new spot formed. The reaction was clean according to TLC. The reaction mixture was diluted with H₂O 500 mL and extracted with EtOAc 300 mL (100 mL*2). The combined organic layers were washed with brine 600 mL (200 mL*3), dried over Na₂SO₄, filtered and concentrated under reduced pressure to give a residue. The residue was purified by column chromatography (SiO₂, Petroleum ether/Ethyl acetate=1/0 to 10/1). 2 (13.9 g, 64.34 mmol, 88% yield) was obtained as a white solid.

¹H NMR (400 MHz, CHLOROFORM-d) δ=6.79 (d, J=8.8 Hz, 1H), 6.25 (d, J=8.8 Hz, 1H), 6.01 (s, 2H), 3.57 (s, 2H)

### General Procedure for Preparation of Intermediate 3:

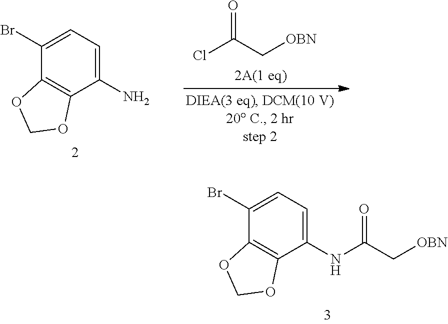

To a solution of 2A (11.88 g, 64.34 mmol, 9.99 mL, 1 eq) and DIEA (24.95 g, 193.03 mmol, 33.62 mL, 3 eq) in DCM (139 mL) was added 2 (13.9 g, 64.34 mmol, 1 eq), The mixture was stirred at 20° C. for 2 hr. TLC (Petroleum ether/Ethyl acetate=3/1, R_{f}=0.31) indicated 2 was consumed completely and one new spot formed. The reaction was clean according to TLC. The mixture was diluted with H₂O 100 mL and extracted with DCM 300 mL (100 mL*3). The combined organic layers were washed with brine 100 mL, dried over Na₂SO₄, filtered and concentrated under reduced pressure to give a residue. The residue was purified by column chromatography (SiO₂, Petroleum ether/Ethyl acetate=1/0 to 10/1). 3 (17 g, 46.68 mmol, 72% yield) was obtained as an off-white solid.

LCMS: RT=1.315 min, MS cal.: 363.01, 365.01, [M+H]⁺=363.8, 365.8

¹H NMR (400 MHz, CHLOROFORM-d) δ=8.31 (s, 1H), 7.64 (d, J=9.2 Hz, 1H), 7.43-7.35 (m, 5H), 6.97 (d, J=9.2 Hz, 1H), 6.08 (s, 2H), 4.67 (s, 2H), 4.11 (s, 2H)

### General Procedure for Preparation of Intermediate 4 & 4A:

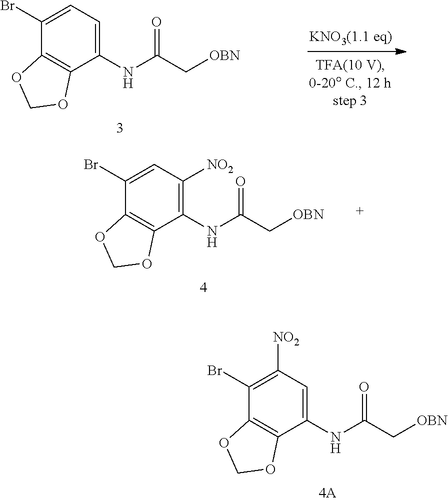

TFA (145 mL) was charged to the three-necked round bottom flask, then 3 (14.5 g, 39.81 mmol, 1 eq) was added to the mixture at 0° C. KNO₃ (4.43 g, 43.80 mmol, 1.1 eq) was added in portions to the reaction mixture at 0° C. After the addition, the mixture was stirred at 20° C. for 12 hr. TLC (Petroleum ether/Ethyl acetate=3/1) indicated 3 was consumed completely and one new spot formed. The reaction mixture was quenched by addition H₂O 250 mL at 25° C., and then extracted with DCM 300 mL (150 mL*2). The combined organic layers were concentrated under reduced pressure to give a residue. The residue was dissolved with ACN 20 mL and added dropwise to sat. NaHCO₃ solution (100 mL) (pH ˜8), extracted with DCM 100 mL (50 mL*2). The combined organic layers were washed with brine 50 mL, dried over Na₂SO₄, filtered and concentrated under reduced pressure to give a residue. The residue was purified by column chromatography (SiO₂, Petroleum ether/Ethyl acetate=1/0 to 5/1). 4 (3.5 g, 8.55 mmol, 21% yield) and 4A (3.5 g, 8.55 mmol, 21% yield) was obtained as an off-white solid.

LCMS: RT=1.297 min, MS cal.: 408.0, 410.0, [M+H]⁺=408.8, 410.8

¹H NMR (400 MHz, CHLOROFORM-d) δ=9.57 (s, 1H), 8.64 (s, 1H), 8.41 (s, 1H), 7.97 (s, 1H), 7.45-7.33 (m, 5H), 6.29-6.21 (m, 2H), 4.73-4.65 (m, 2H), 4.19-4.10 (m, 2H)

### General Procedure for Preparation of Intermediate 5:

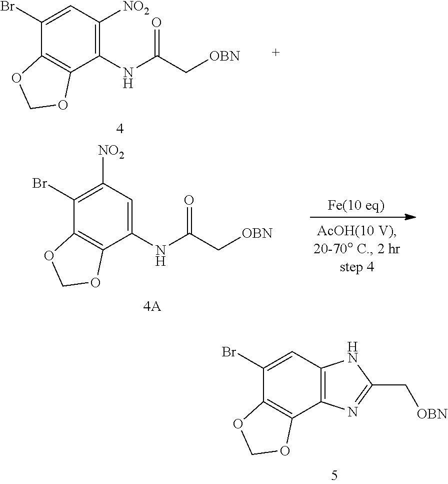

AcOH (88 mL) was charged to the three-necked round bottom flask, then 4 (4.4 g, 10.75 mmol, 1 eq) and 4A (4.4 g, 10.75 mmol, 1 eq) was added to the mixture at 20° C. At 50° C. (inner temperature), Fe (6.00 g, 53.77 mmol, 10 eq) was added in portions to the reaction mixture at 50° C. After the addition, the mixture was stirred at 70° C. for 2 hr. LC-MS showed 4 was consumed completely and one main peak with desired mass was detected. The reaction mixture was filtered through a pad of Celite, and the filter cake was washed with MeOH 90 mL (30 mL*3), the combined filtrate was concentrated under reduced pressure to give a residue. The residue was diluted with H₂O 50 mL and extracted with DCM 150 mL (50 mL*3). The combined organic layers were washed with brine 20 mL, dried over Na₂SO₄, filtered and concentrated under reduced pressure to give a residue. The residue was purified by prep-HPLC (column: Welch Xtimate C18 250*100 mm #10 um; mobile phase: [H₂O (0.1% TFA)-ACN]; gradient: 20%-50% B over 20.0 min). 5 (2.2 g, 6.09 mmol, 56% yield) was obtained as a yellow solid.

LCMS: RT=1.415 min, MS cal.: 360.0, 362.0, [M+H]⁺=360.9, 362.9

¹H NMR (400 MHz, CHLOROFORM-d) δ=7.40-7.32 (m, 5H), 7.18 (s, 1H), 6.14 (s, 2H), 4.82 (s, 2H), 4.65 (s, 2H)

### General Procedure for Preparation of Intermediate 6 & 6A:

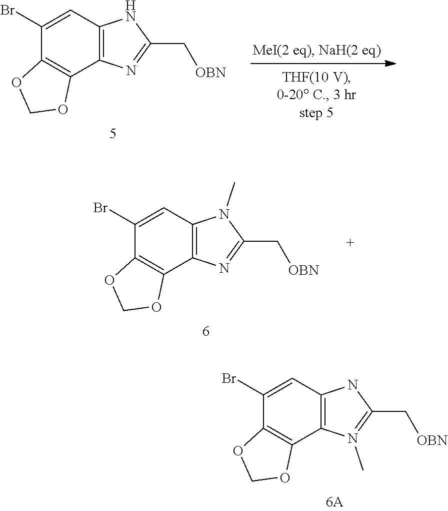

5 (2.2 g, 6.09 mmol, 1 eq) in THF (22 mL) was charged to the three-necked round bottom flask, then NaH (487.28 mg, 12.18 mmol, 60% purity, 2 eq) was added to the mixture at 0° C. and stirred for 30 min at 0° C. At 0° C. (inner temperature), MeI (1.73 g, 12.18 mmol, 758.38 μL, 2 eq) was added in portions to the reaction mixture at 0° C. After the addition, the mixture was stirred at 20° C. for 2.5 hr. LC-MS showed 5 was consumed completely and two main peaks with desired mass were detected. The reaction mixture was quenched by addition H₂O 20 mL at 20° C. and extracted with EtOAc 60 mL (20 mL*3). The combined organic layers were washed with brine 20 mL (10 mL*2), dried over Na₂SO₄, filtered and concentrated under reduced pressure to give a residue. The residue was purified by prep-HPLC (column: Phenomenex luna C18 250*50 mm*15 um; mobile phase: [H₂O (0.1% TFA)-ACN]; gradient: 30%-70% B over 10.0 min). 6 (680 mg, 1.81 mmol, 29% yield) was obtained as a white solid.

LCMS: RT=1.639 min, MS cal.: 374.0, 376.0, [M+H]⁺=374.9, 376.9

¹H NMR (400 MHz, CHLOROFORM-d) δ=7.41-7.28 (m, 5H), 6.97 (s, 1H), 6.17 (s, 2H), 4.82 (s, 2H), 4.57 (s, 2H), 3.77-3.75 (m, 3H)

6A (1.05 g, 2.80 mmol, 45% yield) was obtained as a white solid.

LCMS: RT=1.487 min, MS cal.: 374.03, 376.02, [M+H]⁺=374.9, 376.9

¹H NMR (400 MHz, CHLOROFORM-d) δ=7.41 (s, 1H), 7.38-7.30 (m, 5H), 6.12 (s, 2H), 4.78 (s, 2H), 4.58 (s, 2H), 3.97-3.90 (m, 3H)

### General Procedure for Preparation of Intermediate 7:

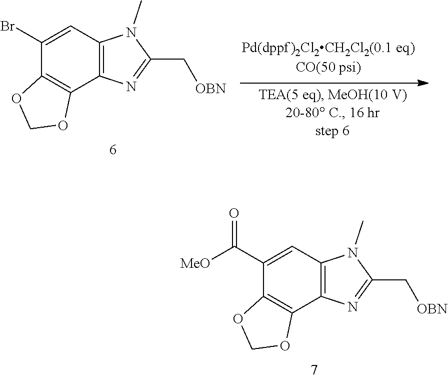

To a solution of 6 (0.58 g, 1.55 mmol, 1 eq) in MeOH (6 mL) was added TEA (782.09 mg, 7.73 mmol, 1.08 mL, 5 eq) and Pd(dppf)Cl₂·CH₂Cl₂ (126.23 mg, 154.58 μmol, 0.1 eq) at 20° C. The suspension was degassed and purged with CO for 3 times. The mixture was stirred under CO (50 Psi) at 80° C. for 16 hr. LC-MS showed 6 was consumed completely and one main peak with desired mass was detected. The reaction mixture was filtered, and the filter cake was washed with EtOAc 10 mL*3. Then the filtrate was diluted with H₂O 30 mL and extracted with EtOAc 60 mL (20 mL*3). The combined organic layers were washed with FA (1M) to pH=5-6 and washed with brine 10 mL, dried over Na₂SO₄, filtered, and concentrated under reduced pressure to give a residue. The residue was purified by column chromatography (SiO₂, Petroleum ether/Ethyl acetate=0/1 to 1/1). 7 (350 mg, 987.71 μmol, 63% yield) was obtained as an off-white solid.

¹H NMR (400 MHz, CHLOROFORM-d) δ=7.48 (s, 1H), 7.38-7.28 (m, 5H), 6.25 (s, 2H), 4.85 (s, 2H), 4.59 (s, 2H), 3.98 (s, 3H), 3.83 (s, 3H)

### General Procedure for Preparation of Intermediate 8:

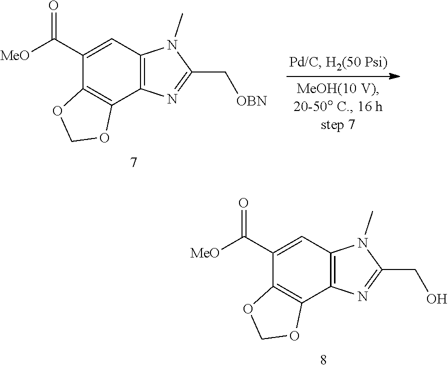

MeOH (5 mL) was charged to the 35 mL hydrogenating flask, then Pd/C (367.89 mg) was added at 20° C. At 20° C. (inner temperature), 7 (350 mg, 987.71 μmol, 1 eq) in MeOH (5 mL) was added to the reaction mixture. The mixture was stirred under H₂ (50 psi) at 50° C. for 16 hr. TLC indicated ˜20% of 7 remained, and one major new spot with lower polarity was detected. The reaction mixture was filtered and concentrated under reduced pressure to give a residue. The crude product was triturated with DCM (3 mL) at 20° C. for 1 min, filtered and the filtered cake was concentrated under reduced pressure to give a residue. 8 (140 mg, 529.83 μmol, 53% yield) was obtained as a white solid.

LCMS: RT=0.706 min, MS cal.: 264.07, [M+H]⁺=265.0

¹H NMR (400 MHz, DMSO-d₆) δ=7.52 (s, 1H), 6.19 (s, 2H), 5.72 (s, 1H), 4.75-4.65 (m, 2H), 3.83-3.82 (m, 1H), 3.83 (d, J=13.2 Hz, 5H), 3.41-3.37 (m, 1H)

### General Procedure for Preparation of Intermediate 9:

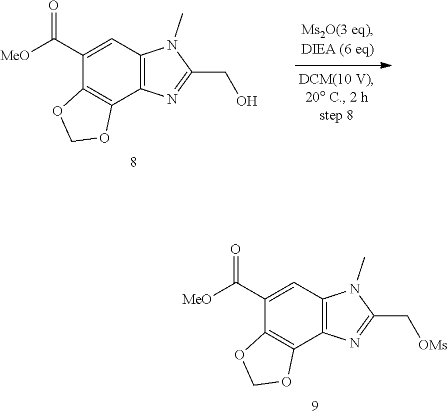 [SMILES: COC(=O)c1cc2c(nc(CO)n2C)c2c1OCO2 ; COC(=O)c1cc2c(nc(COS(C)(=O)=O)n2C)c2c1OCO2]

To a solution of 8 (50 mg, 189.23 μmol, 1 eq) and DIEA (146.73 mg, 1.14 mmol, 197.75 μL, 6 eq) in DCM (1 mL) was added Ms₂O (98.89 mg, 567.68 μmol, 3 eq) at 20° C. The mixture was stirred at 20° C. for 2 hr. TLC (Petroleum ether/Ethyl acetate=0:1, Rr-0.51) indicated 8 was consumed completely and one new spot formed. The reaction was clean according to TLC. The reaction mixture was quenched by addition H₂O 1 mL at 20° C. and extracted with DCM (3 mL*3). The combined organic layers were washed with FA (IM) to pH=5-6, then the organic layer was washed with sat. NaHCO₃ to pH=7-8, and washed with brine 1 mL, dried over Na₂SO₄, filtered and concentrated under reduced pressure to give a residue. 9 (50 mg, crude) was obtained as a yellow oil.

LCMS: RT=1.153 min, MS cal.: 342.05, [M+H]⁺=343.0

¹H NMR (400 MHz, CHLOROFORM-d) δ=7.54 (s, 1H), 6.26 (s, 2H), 5.49 (s, 2H), 3.99 (s, 3H), 3.89 (s, 3H), 3.12 (s, 3H)

### General Procedure for Preparation of Intermediate 10:

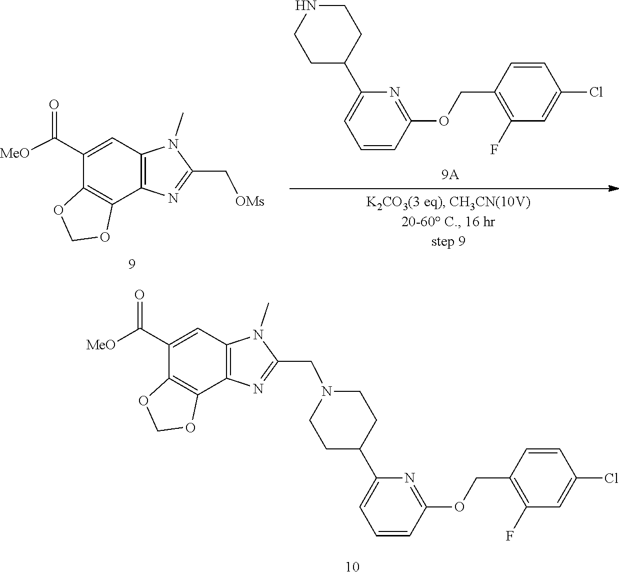 [SMILES: COC(=O)c1cc2c(nc(COS(C)(=O)=O)n2C)c2c1OCO2 ; Fc1cc(Cl)ccc1COc1cccc(C2CCNCC2)n1 ; COC(=O)c1cc2c(nc(CN3CCC(c4cccc(OCc5ccc(Cl)cc5F)n4)CC3)n2C)c2c1OCO2]

To a solution of 9 (50 mg, 146.06 μmol, 1 eq), 9A (46.85 mg, 146.06 μmol, 1 eq) in ACN (1 mL) was added K₂CO₃ (60.56 mg, 438.18 μmol, 3 eq) at 20° C. The mixture was stirred at 60° C. for 16 hr. TLC (Petroleum ether:Ethyl acetate=0:1, Rf=0.14) indicated 9 was consumed completely and one new spot formed. The reaction was clean according to TLC. The reaction mixture was diluted with H₂O 2 mL and extracted with DCM 9 mL (3 mL*3). The combined organic layers were washed with brine 1 mL, dried over Na₂SO₄, filtered, and concentrated under reduced pressure to give residue. The residue was purified by column chromatography (SiO₂, Petroleum ether/Ethyl acetate=1/0 to 0/1). 10 (40 mg, 70.55 μmol, 48.30% yield) was obtained as a yellow oil.

LCMS: RT=1.614 min, MS cal.: 566.17, [M+H]⁺=567.1

1H NMR (400 MHz, CHLOROFORM-d) δ=7.53-7.48 (m, 2H), 7.47-7.41 (m, 1H), 7.15-7.07 (m, 2H), 6.73 (d, J=7.2 Hz, 1H), 6.61 (d, J=8.0 Hz, 1H), 6.23 (s, 2H), 5.41 (s, 2H), 3.98 (s, 3H), 3.93 (s, 3H), 3.85 (s, 2H), 2.98 (d, J=11.2 Hz, 2H), 2.67-2.56 (m, 1H), 2.34-2.25 (m, 2H), 1.92-1.79 (m, 5H), 1.79-1.75 (m, 1H)

### General Procedure for Preparation of Compound 275 (11):

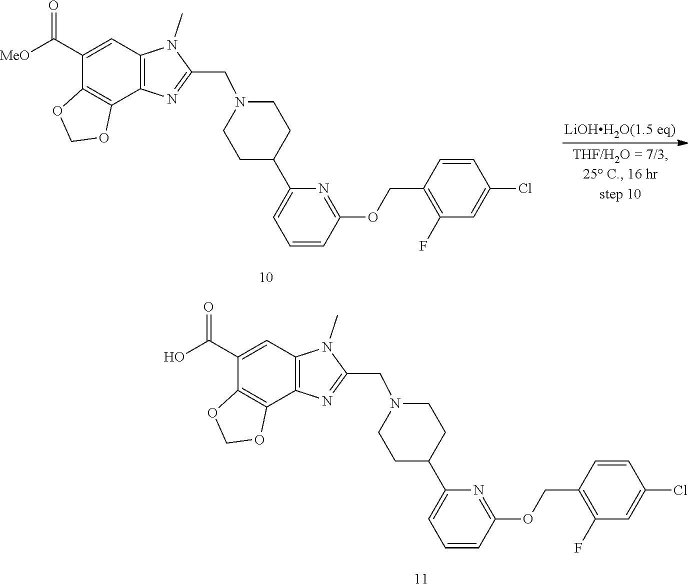 [SMILES: COC(=O)c1cc2c(nc(CN3CCC(c4cccc(OCc5ccc(Cl)cc5F)n4)CC3)n2C)c2c1OCO2 ; Cn1c(CN2CCC(c3cccc(OCc4ccc(Cl)cc4F)n3)CC2)nc2c3c(c(C(=O)O)cc21)OCO3]

To a solution of 10 (45 mg, 79.36 μmol, 1 eq) in THF (0.7 mL) was added LiOH·H₂O (5.00 mg, 119.05 μmol, 1.5 eq) in H₂O (0.3 mL). The mixture was stirred at 25° C. for 16 hr. LC-MS showed ˜6% of 10 remained. Several new peaks were shown on LC-MS and ˜80% of desired compound was detected. The reaction mixture was purified by prep-HPLC (column: Waters Xbridge BEH C18 100*30 mm*10 um; mobile phase: [H₂O (10 mM NH₄HCO₃)-ACN]; gradient: 15%-60% B over 8.0 min). 7-((4-(6-((4-Chloro-2-fluorobenzyl)oxy)pyridin-2-yl)piperidin-1-yl)methyl)-6-methyl-6H-[1,3]dioxolo[4′,5′:3,4]benzo[1,2-d]imidazole-4-carboxylic acid (16.48 mg, 29.30 μmol, 36.92% yield, 98.33% purity) was obtained as a white solid.

LCMS: RT=2.342 min, MS cal.: 552.16, [M+H]⁺=553.1

HPLC: RT=11.066 min, purity: 98.33%

¹H NMR (400 MHz, DMSO-d6) δ=7.66-7.52 (m, 2H), 7.47 (s, 1H), 7.47-7.42 (m, 1H), 7.32-7.25 (m, 1H), 6.86 (d, J=7.2 Hz, 1H), 6.66 (d, J=8.0 Hz, 1H), 6.14 (s, 2H), 5.36 (s, 2H), 3.85 (s, 3H), 3.83-3.75 (m, 2H), 2.97-2.88 (m, 2H), 2.65-2.56 (m, 1H), 2.20 (t, J=10.8 Hz, 2H), 1.85-1.63 (m, 4H)

### Example A241—Preparation of Compound 274. (S)-2-((4-(2-(4-Cyano-2-fluorophenyl)-2,3-dihydrobenzo[b][1,4]dioxin-5-yl)piperidin-1-yl)methyl)-4-methoxy-1-methyl-1H-benzo[d]imidazole-6-carboxylic acid (Compound 274) (3)

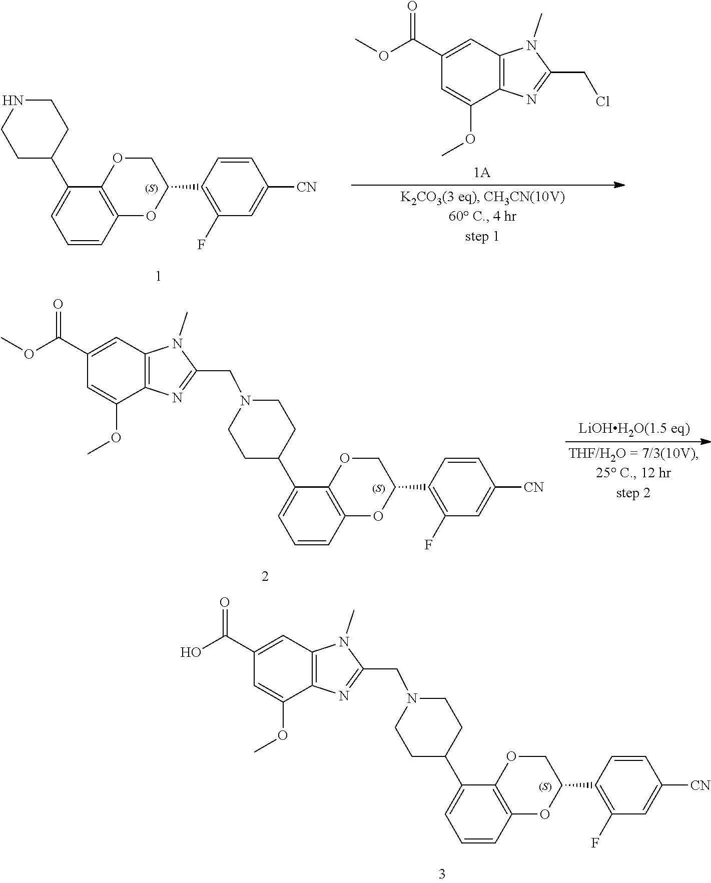

### General Procedure for Preparation of Intermediate 2:

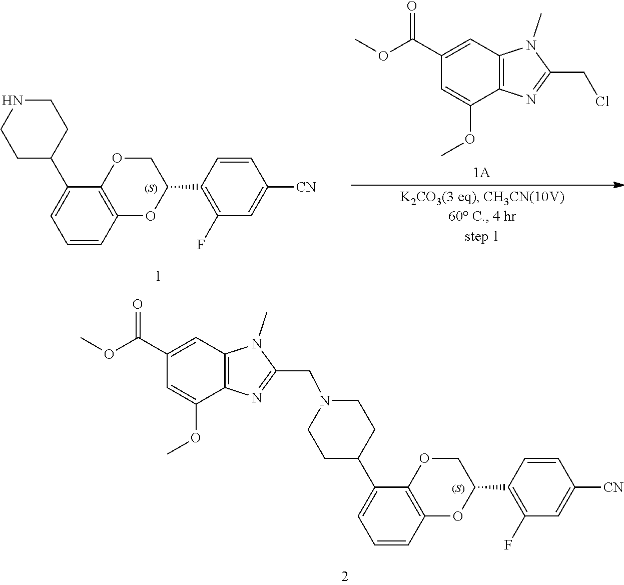

To a solution of 1 (55.00 mg, 121.57 μmol, 1 eq, TFA) and 1A (35.93 mg, 133.73 μmol, 1.1 eq) in ACN (0.55 mL) was added K₂CO₃ (50.41 mg, 364.72 μmol, 3 eq). The mixture was stirred at 60° C. for 4 hr. LCMS showed 1 was consumed and one main peak with desired mass was detected. The residue was diluted with H₂O (1 mL) and extracted with EtOAc (3 mL*3). The combined organic layers were washed with brine (2 mL*2), dried over Na₂SO₄, filtered and concentrated under reduced pressure to give a residue. The residue was purified by column chromatography (SiO₂, Petroleum ether/Ethyl acetate=1/0 to 0/1). 2 (60 mg, 105.15 μmol, 86.49% yield) was obtained as light-yellow oil.

LCMS: RT=1.530 min, MS cal.: 570.6, 571.6, [M+H]⁺=571.1, 572.2

¹H NMR (400 MHz, CHLOROFORM-d) δ=7.78 (d, J=0.8 Hz, 1H), 7.68 (t, J=7.4 Hz, 1H), 7.56-7.52 (m, 1H), 7.43 (dd, J=9.8, 1.2 Hz, 1H), 7.39 (s, 1H), 6.90-6.82 (m, 3H), 5.52-5.46 (m, 1H), 4.51 (dd, J=11.4, 2.2 Hz, 1H), 4.07 (s, 3H), 3.99-3.94 (m, 7H), 3.90-3.86 (m, 2H), 3.01-2.89 (m, 3H), 2.36-2.24 (m, 2H), 1.85 (br d, J=12.0 Hz, 2H), 1.69 (dt, J=12.0, 3.2 Hz, 2H

### General Procedure for Preparation of Compound 274 (3):

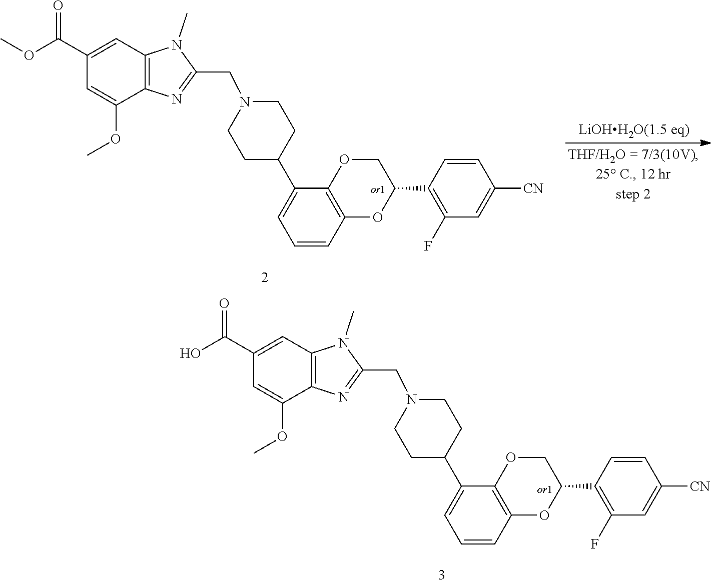

To a solution of 2 (60 mg, 105.15 μmol, 1 eq) in THF (0.42 mL) and H₂O (0.18 mL) was added LiOH·H₂O (6.62 mg, 157.73 μmol, 1.5 eq). The mixture was stirred at 25° C. for 12 hr. LCMS showed 2 was consumed and desired mass was detected. The compound was purified directly. The residue was purified by Prep-HPLC (column: Waters Xbridge Prep OBD C18 150*40 mm*10 um; mobile phase: [H₂O (10 mM NH₄HCO₃)-ACN]; gradient: 15%-55% B over 8.0 min). (S)-2-((4-(2-(4-Cyano-2-fluorophenyl)-2,3-dihydrobenzo[b][1,4]dioxin-5-yl)piperidin-1-yl)methyl)-4-methoxy-1-methyl-1H-benzo[d]imidazole-6-carboxylic acid (27.52 mg, 49.26 μmol, 46.85% yield, 99.63% purity) was obtained as a white solid.

LCMS: RT=2.584 min, MS cal.: 556.6, 557.6, [M+H]⁺=557.3, 558.3

HPLC: RT=10.318 min, purity: 99.63%

¹H NMR (400 MHz, DMSO-d6) δ=7.97 (dd, J=10.0, 1.0 Hz, 1H), 7.79 (dd, J=8.0, 1.0 Hz, 1H), 7.75 (s, 1H), 7.72-7.67 (m, 1H), 7.27 (s, 1H), 6.87-6.80 (m, 3H), 5.55 (dd, J=7.6, 2.0 Hz, 1H), 4.55 (dd, J=11.4, 2.2 Hz, 1H), 4.14 (dd, J=11.6, 7.6 Hz, 1H), 3.94 (s, 3H), 3.88 (s, 3H), 3.79 (s, 2H), 2.94 (d, J=11.0 Hz, 2H), 2.88-2.82 (m, 1H), 2.18 (t, J=10.6 Hz, 2H), 1.76-1.58 (m, 3H)

### Example A242—Preparation of Compound 273. 4-(((6-(1-((4-ethoxy-1-methyl-6-(1H-tetrazol-5-yl)-1H-benzo[d]imidazol-2-yl)methyl)piperidin-4-yl)pyridin-2-yl)oxy)methyl)-3-fluorobenzonitrile (Compound 273) (5)

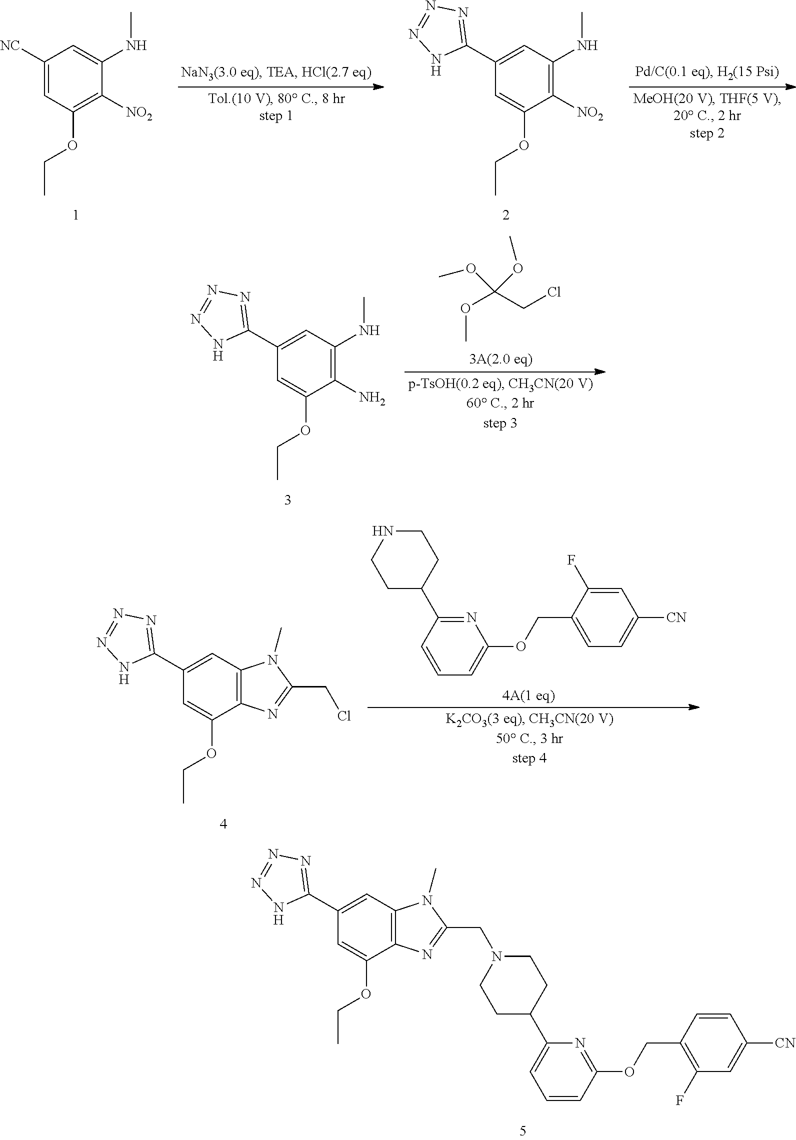

### General Procedure for Preparation of Intermediate 2:

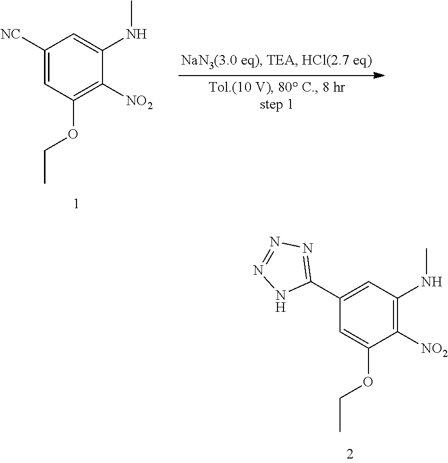 [SMILES: CCOc1cc(C#N)cc(NC)c1[N+](=O)[O-] ; CCOc1cc(-c2nnn[nH]2)cc(NC)c1[N+](=O)[O-]]

To a solution of 1 (900 mg, 2.44 mmol, 1 eq) and TEA·HCl (504.03 mg, 3.66 mmol, 1.5 eq) in Tol. (9 mL) was added NaN₃ (238.04 mg, 3.66 mmol, 1.5 eq) in portions under N₂ atmosphere. After the addition, the mixture was stirred at 80° C. for 4 hr. LC-MS showed ˜30% of 1 remained, and then TEA·HCl (403.22 mg, 2.93 mmol, 1.2 eq) and NaN₃ (238.04 mg, 3.66 mmol, 1.5 eq) was added to the reaction mixture. The mixture was stirred at 80° C. for another 4 hr. LC-MS showed ˜7% of 1 remained, and several new peaks were showed on LC-MS and ˜31% of desired compound was detected. The reaction mixture was cooled to 20° C., then TEA was added to the mixture until pH>12, which was purified directly by column chromatography (SiO₂, Ethyl acetate/MeOH=1/0 to 1/1). 2 (0.8 g, 2.12 mmol, 86.82% yield) was obtained as a yellow oil.

¹H NMR (400 MHz, CDCl₃) δ=7.25 (d, J=1.6 Hz, 1H), 7.16 (d, J=0.8 Hz, 1H), 6.42-6.41 (m, 1H), 4.27-4.18 (m, 2H), 3.03-2.99 (m, 3H), 1.50-1.36 (m, 3H)

### General Procedure for Preparation of Intermediate 3:

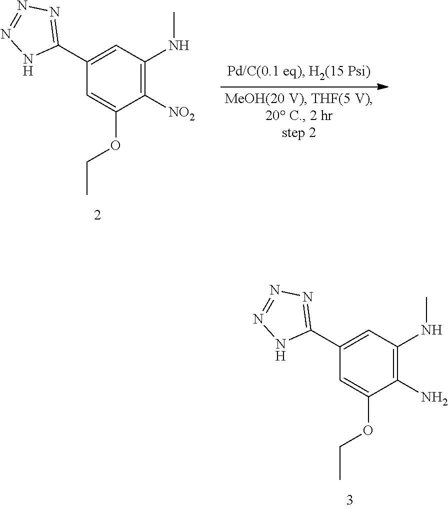 [SMILES: CCOc1cc(-c2nnn[nH]2)cc(NC)c1[N+](=O)[O-] ; CCOc1cc(-c2nnn[nH]2)cc(NC)c1N]

To a solution of Pd/C (122.05 mg, 114.69 μmol, 10% purity, 0.1 eq) in MeOH (4 mL) was added 2 (400 mg, 1.06 mmol, 1 eq) in MeOH (4 mL) and THF (2 mL) at 20° C. under N₂ atmosphere. After addition, the mixture was degassed and purged with H₂ for three times, then stirred under H₂ atmosphere (15 Psi) at 20° C. for 2 hr. LC-MS showed 2 was consumed completely and desired mass was detected. The reaction was filtered through a Celite pad and washed by MeOH (10 mL*3), and the filtrate was concentrated under reduced pressure to give a residue. The residue was purified by column chromatography (SiO₂, Petroleum ether/Ethyl acetate=1/1 to 0/1 and then DCM/MeOH=10/1). 3 (300 mg, 819.61 μmol, 77.35% yield) was obtained as a yellow oil.

¹H NMR (400 MHz, CDCl₃) δ=7.22 (d, J=1.6 Hz, 1H), 7.17 (d, J=1.6 Hz, 1H), 3.99-3.87 (m, 2H), 2.75 (s, 3H), 1.39-1.26 (m, 3H)

### General Procedure for Preparation of Intermediate 4:

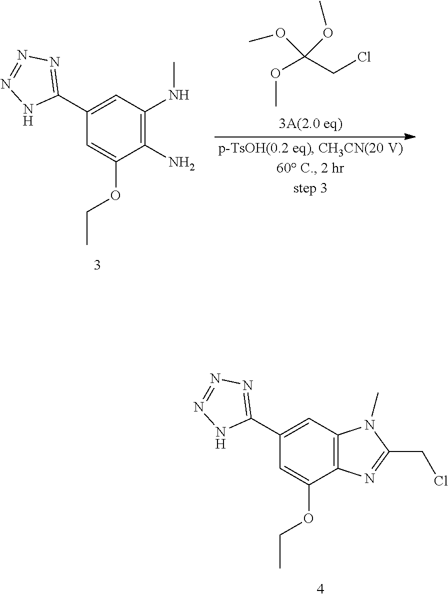

To a solution of 3 (200 mg, 853.76 μmol, 1 eq) in MeCN (4 mL) was added 3A (263.97 mg, 1.71 mmol, 230.14 μL, 2 eq) and p-TsOH (29.40 mg, 170.75 μmol, 0.2 eq) at 20° C. After addition, the mixture was stirred at 60° C. for 2 hr. LC-MS showed 3 was consumed completely and desired mass was detected. The reaction mixture was filtered. And the filter cake was dried under reduced pressure to give the crude product. 4 (170 mg, crude) was obtained as a yellow solid.

¹H NMR (400 MHz, DMSO-d6) δ=7.91 (d, J=0.8 Hz, 1H), 7.43 (d, J=1.2 Hz, 1H), 5.12 (s, 2H), 4.42-4.28 (m, 2H), 3.91 (m, 3H), 1.55-1.37 (m, 3H)

### General Procedure for Preparation of Compound 273 (5):

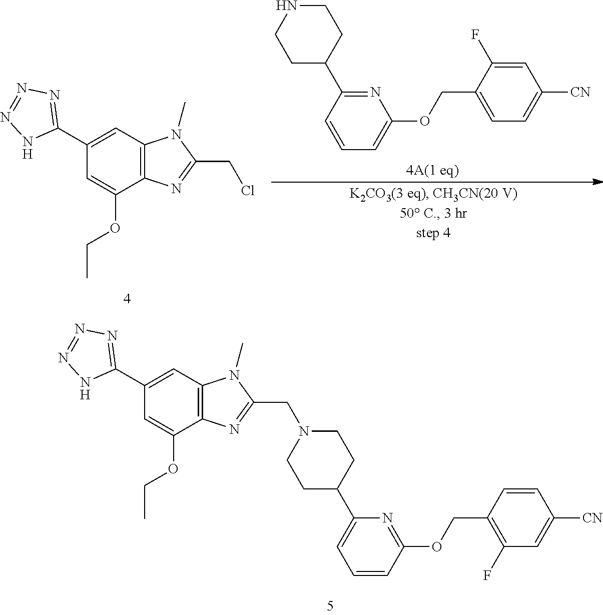 [SMILES: CCOc1cc(-c2nnn[nH]2)cc2c1nc(CCl)n2C ; N#Cc1ccc(COc2cccc(C3CCNCC3)n2)c(F)c1 ; CCOc1cc(-c2nnn[nH]2)cc2c1nc(CN1CCC(c3cccc(OCc4ccc(C#N)cc4F)n3)CC1)n2C]

To a solution of 4 (170 mg, 580.75 μmol, 1 eq), 4A (235.87 mg, 580.75 μmol, 1 eq, 2.6 HCl) in MeCN (3.4 mL) was added K₂CO₃ (240.80 mg, 1.74 mmol, 3 eq) at 20° C. After addition, the mixture was stirred at 50° C. for 3 hr. LC-MS showed 4 was consumed completely and desired mass was detected. The reaction mixture was filtered. The filtrate was purified by prep-HPLC (neutral condition; column: 2 Phenomenex Gemini C18 75*40 mm*3 um; mobile phase: [H₂O (10 mM NH₄HCO₃)-ACN]; gradient: 20%-45% B over 8.0 min). 4-(((6-(1-((4-ethoxy-1-methyl-6-(1H-tetrazol-5-yl)-1H-benzo[d]imidazol-2-yl)methyl)piperidin-4-yl)pyridin-2-yl)oxy)methyl)-3-fluorobenzonitrile (29.67 mg, 50.49 μmol, 8.69% yield, 96.60% purity) was obtained as a white solid.

LCMS: RT=2.656 min, MS cal.: 567.3, [M+H]⁺=568.3

HPLC: RT=9.893 min, purity: 96.60%

¹H NMR (400 MHz, DMSO-d6) δ=7.87 (d, J=10.0 Hz, 1H), 7.85-7.81 (m, 1H), 7.72-7.67 (m, 2H), 7.67-7.61 (m, 1H), 7.38 (d, J=1.2 Hz, 1H), 6.88 (d, J=7.2 Hz, 1H), 6.75-6.66 (d, J=7.2 Hz, 1H), 5.45 (s, 2H), 4.38-4.22 (m, 2H), 3.97-3.89 (s, 3H), 3.88-3.82 (s, 2H), 3.02-2.89 (m, 2H), 2.62-2.56 (m, 1H), 2.29-2.16 (m, 2H), 1.84-1.61 (m, 4H), 1.50-1.36 (m, 3H)

### Example A243—Preparation of Compound 272. 2-((4-(6-((4-Chloro-2,5-difluorobenzyl)oxy)pyridin-2-yl)piperidin-1-yl)methyl)-4-methoxy-1-methyl-1H-benzo[d]imidazole-6-carboxylic acid (Compound 272) (6)

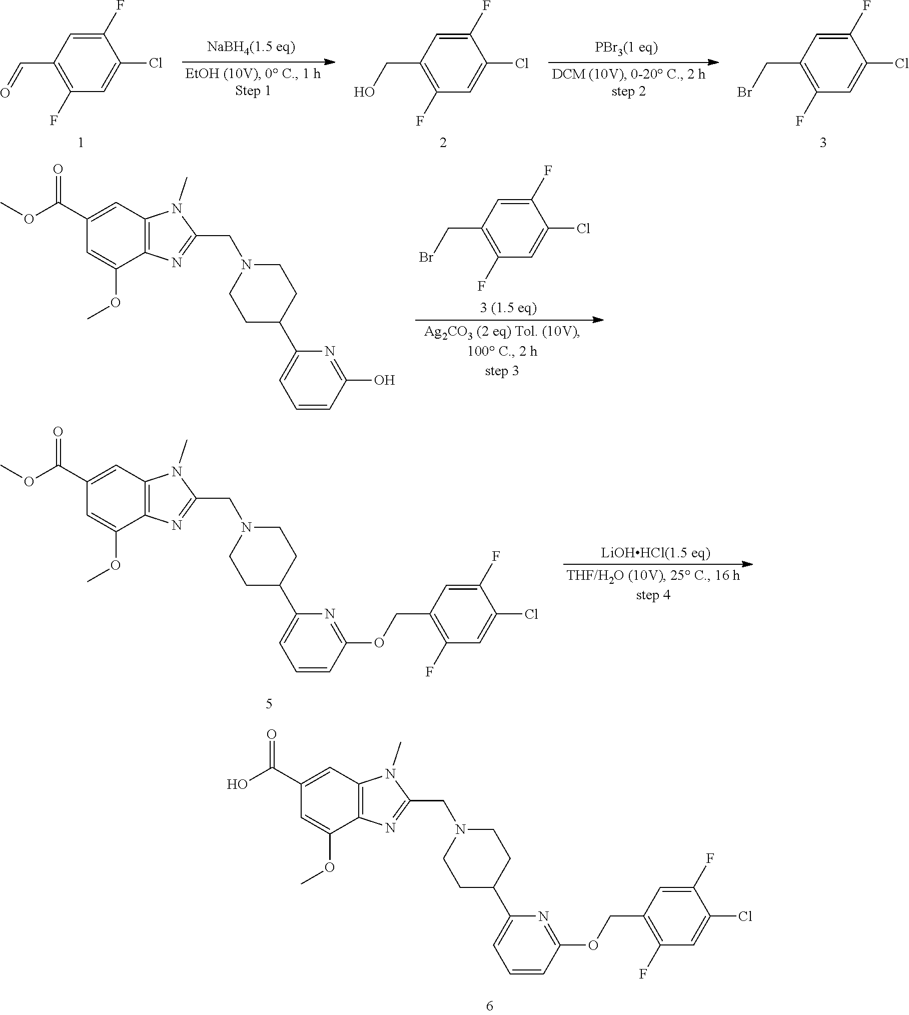

### General Procedure for Preparation of Intermediate 2:

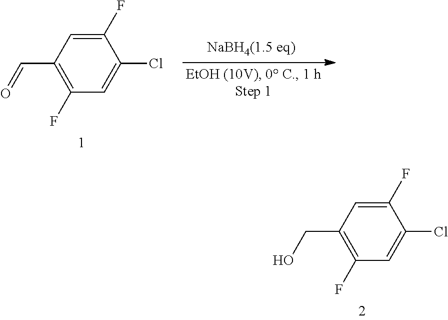 [SMILES: O=Cc1cc(F)c(Cl)cc1F ; OCc1cc(F)c(Cl)cc1F]

1 (500 mg, 2.83 mmol, 1 eq) in EtOH (5 mL) was charged to the three-necked round bottom flask, then NaBH₄ (160.71 mg, 4.25 mmol, 1.5 eq) was added to the mixture at 0° C. After the addition, the mixture was stirred at 0° C. for 1 hr. TLC (Petroleum ether:Ethyl acetate=5:1, Rf=0.34) indicated 1 was consumed completely and one new spot formed. The reaction was clean according to TLC. The reaction mixture was quenched by addition H₂O 5 mL at 0° C., and then extracted with EtOAc 15 mL (5 mL*3). The combined organic layers were washed with brine 5 mL (5 mL*1), dried over Na₂SO₄, filtered and concentrated under reduced pressure to give a residue. 2 (490 mg, 2.63 mmol, 93.02% yield, 96% purity) was obtained as a white solid.

LCMS: RT=1.039 min, MS cal.: 178.00, [M−H]⁻=176.9

1H NMR (400 MHz, CHLOROFORM-d) δ=7.31-7.28 (m, 1H), 7.16-7.11 (m, 1H), 4.75 (s, 2H)

### General Procedure for Preparation of Intermediate 3:

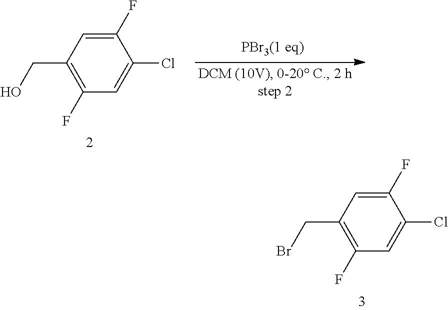 [SMILES: OCc1cc(F)c(Cl)cc1F ; Fc1cc(CBr)c(F)cc1Cl]

DCM (2 mL) was charged to the three-necked round bottom flask, then 2 (200 mg, 1.12 mmol, 1 eq) was added to the mixture at 20° C. PBr₃ (303.19 mg, 1.12 mmol, 1 eq) was added dropwise/in portions/to the reaction mixture at 0° C. After the addition, the mixture was stirred at 0° C. for 2 hr. TLC (Petroleum ether:Ethyl acetate=3:1, Rf=0.58) indicated 2 was consumed completely and one new spot formed. The reaction was clean according to TLC. The reaction mixture was quenched by addition sat. NaHCO₃ 2 mL at 20° C., and then extracted with DCM 6 mL (2 mL*3). The combined organic layers were washed with brine 2 mL, dried over Na₂SO₄, filtered, and concentrated under reduced pressure to give a residue at 20° C. 3 (180 mg, crude) was obtained as a yellow oil.

¹H NMR (400 MHz, CHLOROFORM-d) δ=7.25-7.13 (m, 2H), 4.44 (s, 2H)

### General Procedure for Preparation of Intermediate 5:

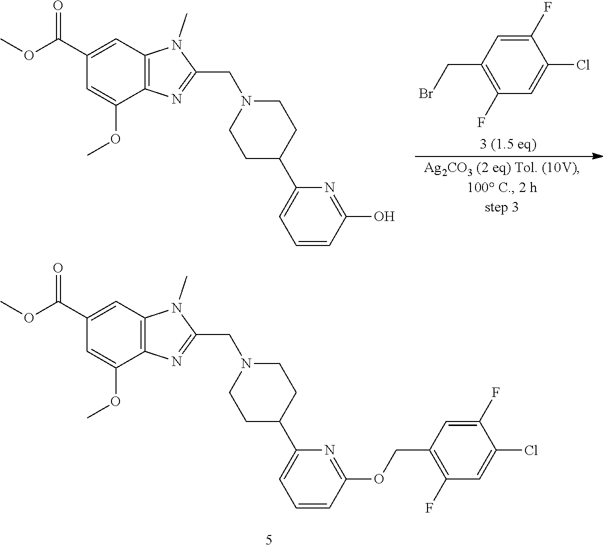 [SMILES: COC(=O)c1cc(OC)c2nc(CN3CCC(c4cccc(O)n4)CC3)n(C)c2c1 ; Fc1cc(CBr)c(F)cc1Cl ; COC(=O)c1cc(OC)c2nc(CN3CCC(c4cccc(OCc5cc(F)c(Cl)cc5F)n4)CC3)n(C)c2c1]

A mixture of 4 (100 mg, 243.63 μmol, 1 eq), 3 (88.24 mg, 365.44 μmol, 1.5 eq), Ag₂CO₃ (134.35 mg, 487.25 μmol, 22.11 μL, 2 eq) in Tol. (1 mL) was degassed and purged with N₂ for 3 times, and then the mixture was stirred at 100° C. for 2 hr under N₂ atmosphere. TLC (Petroleum ether: Ethyl acetate=0:1, R_{f}=0.11) indicated 4 was consumed completely and one new spot formed. The reaction was clean according to TLC. The reaction mixture was diluted with H₂O 10 mL and extracted with EtOAc 30 mL (10 mL*3). The combined organic layers were washed with brine 10 mL, dried over Na₂SO₄, filtered and concentrated under reduced pressure to give a residue. The residue was purified by column chromatography (SiO₂, Petroleum ether/Ethyl acetate=10/1 to 0/1). 5 (45 mg, 78.81 μmol, 32.35% yield) was obtained as a yellow solid.

LCMS: RT=1.697 min, MS cal.: 570.18, [M+H]⁺=571.1

1H NMR (400 MHz, CHLOROFORM-d) δ=7.79 (s, 1H), 7.52 (t, J=7.6 Hz, 1H), 7.43-7.38 (m, 1H), 7.37-7.29 (m, 1H), 7.18-7.11 (m, 1H), 6.75 (d, J=7.2 Hz, 1H), 6.62 (d, J=8.0 Hz, 1H), 5.44-5.36 (m, 2H), 4.07 (s, 3H), 3.97 (s, 6H), 3.94-3.83 (m, 2H), 3.04-2.91 (m, 2H), 2.61 (s, 1H), 2.28 (s, 2H), 2.05 (s, 1H), 1.93-1.73 (m, 4H)

### General Procedure for Preparation of Compound 272 (6):

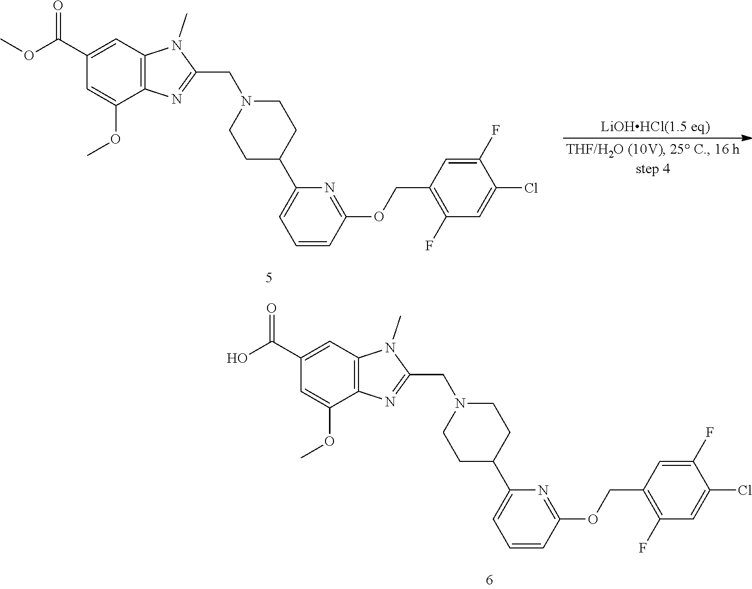 [SMILES: COC(=O)c1cc(OC)c2nc(CN3CCC(c4cccc(OCc5cc(F)c(Cl)cc5F)n4)CC3)n(C)c2c1 ; COc1cc(C(=O)O)cc2c1nc(CN1CCC(c3cccc(OCc4cc(F)c(Cl)cc4F)n3)CC1)n2C]

To a solution of 5 (55 mg, 96.32 μmol, 1 eq) in THF (0.7 mL) was added LiOH·H₂O (6.06 mg, 144.48 μmol, 1.5 eq) in H₂O (0.3 mL). The mixture was stirred at 25° C. for 16 hr. LC-MS showed ˜16% of 5 remained. Several new peaks were shown on LC-MS and ˜67% of desired compound was detected. The reaction mixture was purified by prep-HPLC (column: 2 Phenomenex Gemini C18 75*40 mm*3 um; mobile phase: [H₂O (10 mM NH₄HCO₃)-ACN]; gradient: 25%-55% B over 8.0 min). 2-((4-(6-((4-Chloro-2,5-difluorobenzyl)oxy)pyridin-2-yl)piperidin-1-yl)methyl)-4-methoxy-1-methyl-1H-benzo[d]imidazole-6-carboxylic acid (26.06 mg, 46.30 μmol, 48.07% yield, 98.96% purity) was obtained as a white solid.

LCMS: RT=2.409 min, MS cal.: 556.17, [M+H]⁺=557.2

HPLC: RT=11.417 min, purity: 98.96%

¹H NMR (400 MHz, DMSO-d6) δ=7.73-7.70 (m, 1H), 7.70-7.56 (m, 3H), 7.28 (d, J=0.8 Hz, 1H), 6.91-6.85 (m, 1H), 6.72-6.66 (m, 1H), 5.34 (s, 2H), 3.93 (s, 3H), 3.88 (s, 3H), 3.78 (s, 2H), 2.92 (d, J=11.2 Hz, 2H), 2.64-2.52 (m, 1H), 2.17 (t, J=10.4 Hz, 2H), 1.82-1.62 (m, 4H)

### Example A244—Preparation of Compound 270. (S)-2-((4-(2-(4-Chloro-2-fluorophenyl)-2,3-dihydrobenzo[b][1,4]dioxin-5-yl)piperidin-1-yl)methyl)-4-ethoxy-1-methyl-1H-benzo[d]imidazole-6-carboxylic acid (Compound 270) (3)

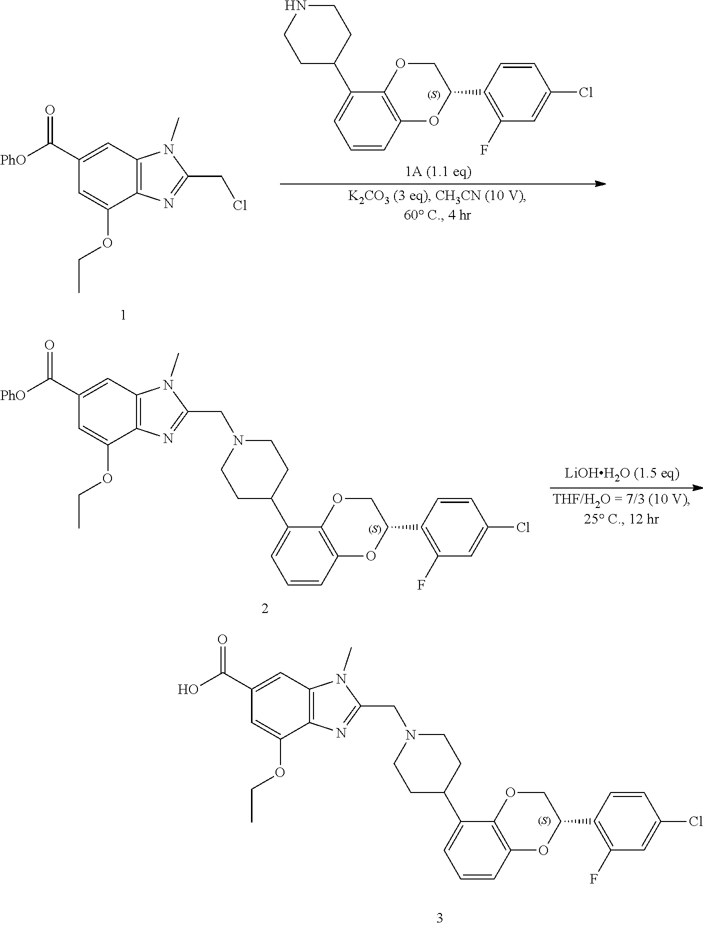

### General Procedure for Preparation of Intermediate 2:

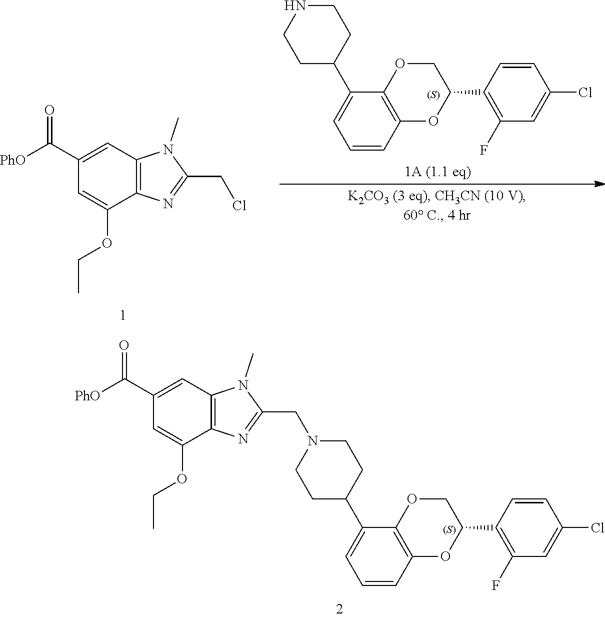

To a solution of 1 (100 mg, 290.03 μmol, 1 eq) and 1A (110.96 mg, 319.03 μmol, 1.1 eq) in CH₃CN (1 mL) was added K₂CO₃ (120.26 mg, 870.09 μmol, 3 eq) at 25° C. The mixture was stirred at 60° C. for 4 hr. LC-MS showed 1 was consumed completely and one main peak with desired mass was detected. The residue was poured into water (10 mL). The aqueous phase was extracted with EA (10 mL*3). The combined organic phase was washed with brine (10 mL*2), dried with anhydrous Na₂SO₄, filtered and concentrated in vacuum. The residue was purified by column chromatography (SiO₂, PE/EA=1/0 to 0/1). 2 (120 mg, 182.89 μmol, 63.06% yield) was a yellow solid.

¹H NMR (400 MHz, CHLOROFORM-d) δ=7.94 (s, 1H), 7.53 (d, J=1.2 Hz, 1H), 7.50-7.43 (m, 3H), 7.30 (t, J=7.2 Hz, 1H), 7.27-7.13 (m, 4H), 6.92-6.78 (m, 3H), 5.41 (dd, J=2.0, 8.4 Hz, 1H), 4.47 (dd, J=2.4, 11.2 Hz, 1H), 4.42-4.29 (m, 2H), 4.05-3.90 (m, 5H), 2.96 (s, 3H), 2.33 (s, 2H), 1.92-1.68 (m, 4H), 1.59 (t, J=7.2 Hz, 3H)

### General Procedure for Preparation of Compound 270 (3):

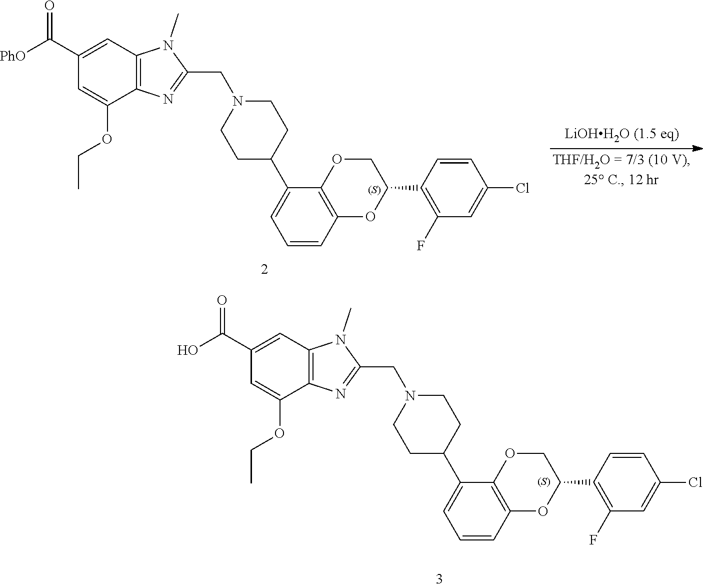

To a solution of 2 (100.00 mg, 152.41 μmol, 1 eq) in THF (0.7 mL) and H₂O (0.3 mL) was added LiOH·H₂O (9.59 mg, 228.61 μmol, 1.5 eq). The mixture was stirred at 25° C. for 12 hr. LC-MS showed ˜24% of 2 remained. Several new peaks were shown on LC-MS and ˜62% of desired compound was detected. The mixture was filtered. The residue was purified by prep-HPLC (column: Waters Xbridge BEH C18 100*30 mm*10 um; mobile phase: [H₂O (10 mM NH₄HCO₃)-ACN]; gradient: 35%-65% B over 8.0 min). (S)-2-((4-(2-(4-Chloro-2-fluorophenyl)-2,3-dihydrobenzo[b][1,4]dioxin-5-yl)piperidin-1-yl)methyl)-4-ethoxy-1-methyl-1H-benzo[d]imidazole-6-carboxylic acid (25.05 mg, 43.19 μmol, 28.34% yield) was a white solid.

LCMS: Rt=2.880 min, MS cal.: 579.19, [M+H]⁺=580.2

¹H NMR (400 MHz, DMSO-d6) δ=7.76 (s, 1H), 7.58-7.51 (m, 2H), 7.39 (dd, J=1.6, 8.4 Hz, 1H), 7.25 (s, 1H), 6.85-6.78 (m, 3H), 5.43 (dd, J=2.0, 8.0 Hz, 1H), 4.51 (dd, J=2.0, 11.2 Hz, 1H), 4.24 (q, J=7.2 Hz, 2H), 4.13 (dd, J=8.4, 11.2 Hz, 1H), 3.89 (s, 3H), 3.34 (s, 3H), 3.00-2.82 (m, 3H), 2.19 (t, J=10.8 Hz, 2H), 1.78-1.55 (m, 4H), 1.41 (t, J=6.8 Hz, 3H)

### Example A245—Preparation of Compound 269. (R)-2-((4-(2-(4-chloro-2-fluorophenyl)-2,3-dihydrobenzo[b][1,4]dioxin-5-yl)piperidin-1-yl)methyl)-4-ethoxy-1-methyl-1H-benzo[d]imidazole-6-carboxylic acid (Compound 269) (3)

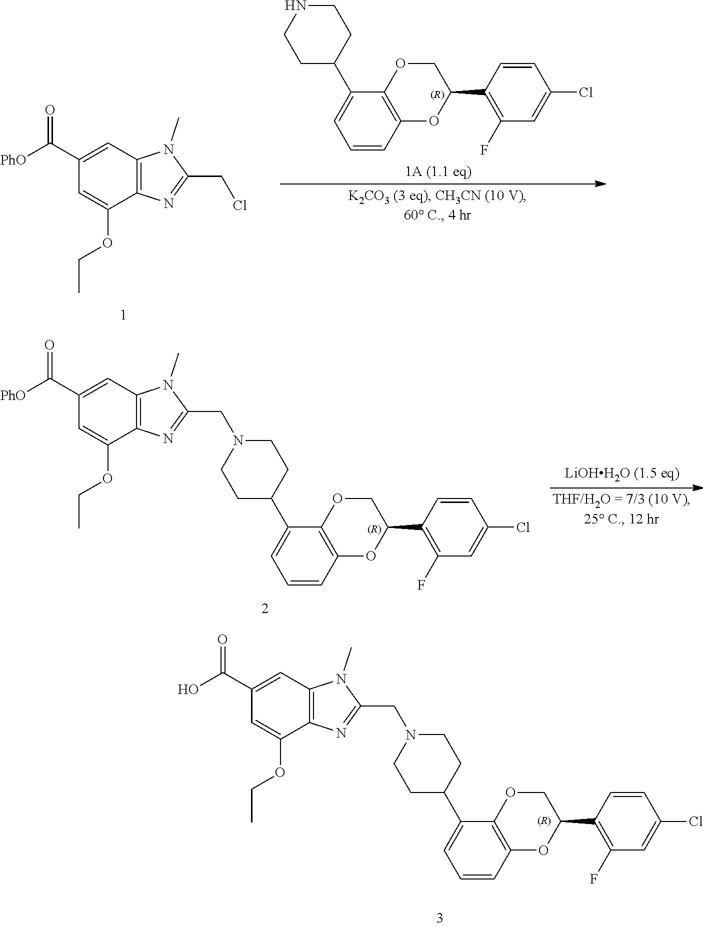

### General Procedure for Preparation of Intermediate 2:

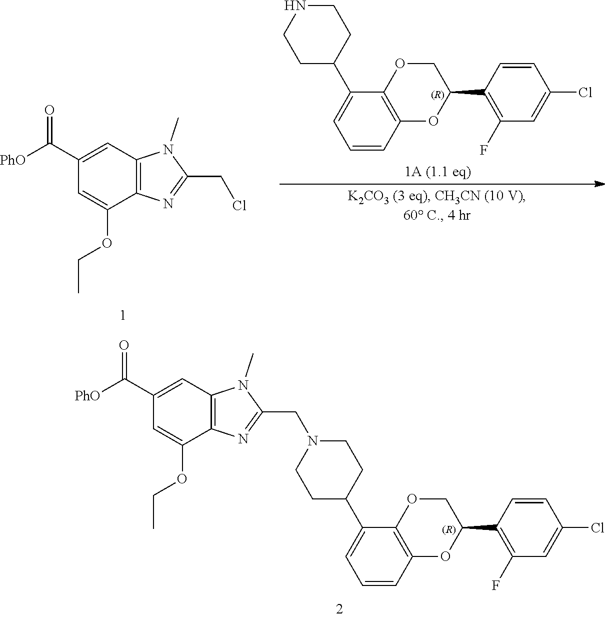

To a solution of 3 (90 mg, 194.88 μmol, 1 eq, TFA) in ACN (1 mL) was added K₂CO₃ (80.80 mg, 584.63 μmol, 3 eq) and 3A (67.19 mg, 194.88 μmol, 1 eq). The mixture was stirred at 60° C. for 2 hr. LCMS showed 3 was consumed completely and one main peak with desired mass was detected. The reaction mixture was diluted with H₂O 10 mL and extracted with EA 30 mL (10 mL*3). The combined organic layers were washed with brine 20 mL, dried over Na₂SO₄, filtered, and concentrated under reduced pressure to give a residue. The residue was purified by column chromatography (SiO₂, PE/EA=1/0 to 1/1). 4 (90 mg, 127.56 μmol, 65.46% yield) was obtained as a yellow solid.

LCMS: RT=1.892 min, MS cal.: 655.2/656.2, [M+H]⁺=656.2/657.2

¹HNMR (400 MHz, DMSO-d6) δ=8.02 (d, J=1.2 Hz, 1H), 7.58-7.46 (m, 4H), 7.41-7.37 (m, 2H), 7.35-7.27 (m, 3H), 6.85-6.79 (m, 3H), 5.46-5.41 (m, 1H), 4.55-4.48 (m, 1H), 4.30 (q, J=7.2 Hz, 2H), 4.13 (dd, J=8.4, 11.2 Hz, 1H), 3.96 (s, 3H), 3.84 (s, 2H), 2.99-2.81 (m, 3H), 2.25-2.16 (m, 2H), 1.78-1.56 (m, 4H), 1.43 (t, J=6.8 Hz, 3H)

### General Procedure for Preparation of Compound 269 (3):

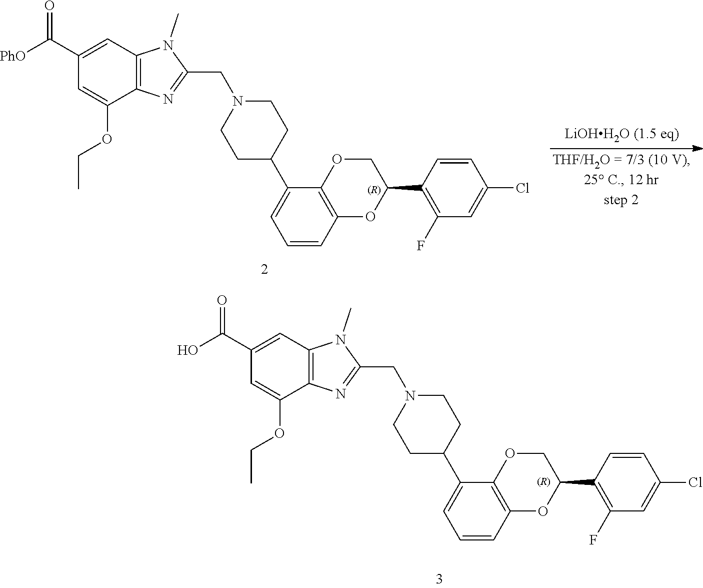

To a solution of 4 (80 mg, 121.93 μmol, 1 eq) in THF (0.7 mL) and H₂O (0.3 mL) was added LiOH·H₂O (7.67 mg, 182.89 μmol, 1.5 eq). The mixture was stirred at 25° C. for 12 hr. LCMS showed 19% of 2 remained. Several new peaks were shown on LCMS and 67% of desired compound was detected. The mixture was used directly, no work-up. The residue was purified by prep-HPLC (neutral condition: column: Waters Xbridge Prep OBD C18 150*40 mm*10 um; mobile phase: [H₂O (10 mM NH₄HCO₃)-ACN]; gradient: 45%-85% B over 8.0 min). (R)-2-((4-(2-(4-Chloro-2-fluorophenyl)-2,3-dihydrobenzo[b][1,4]dioxin-5-yl)piperidin-1-yl)methyl)-4-ethoxy-1-methyl-1H-benzo[d]imidazole-6-carboxylic acid (26.53 mg, 45.28 μmol, 37.14% total yield) was obtained as a white solid.

LCMS: RT=2.872 min, MS cal.: 579.2/580.2, [M+H]⁺=580.2/581.2

HPLC: RT=12.611 min

¹HNMR (400 MHz, DMSO-d6) δ=7.75 (s, 1H), 7.58-7.50 (m, 2H), 7.38 (dd, J=1.6, 8.4 Hz, 1H), 7.25 (d, J=1.2 Hz, 1H), 6.85-6.79 (m, 3H), 5.46-5.39 (m, 1H), 4.55-4.47 (m, 1H), 4.28-4.21 (m, 2H), 4.17-4.09 (m, 1H), 3.92-3.86 (m, 3H), 3.82-3.77 (m, 2H), 2.97-2.81 (m, 3H), 2.19 (t, J=10.8 Hz, 2H), 1.77-1.54 (m, 4H), 1.41 (t, J=6.8 Hz, 3H)

### Example A246—Preparation of Compound 267 and Compound 268. (S)-2-((4-(3-(4-Chloro-2-fluorophenyl)-2,3-dihydrobenzo[b][1,4]dioxin-5-yl)piperidin-1-yl)methyl)-4-methoxy-1-methyl-1H-benzo[d]imidazole-6-carboxylic acid (Compound 268) (3A) & (R)-2-((4-(3-(4-Chloro-2-fluorophenyl)-2,3-dihydrobenzo[b][1,4]dioxin-5-yl)piperidin-1-yl)methyl)-4-methoxy-1-methyl-1H-benzo[d]imidazole-6-carboxylic acid (Compound 267) (3B)

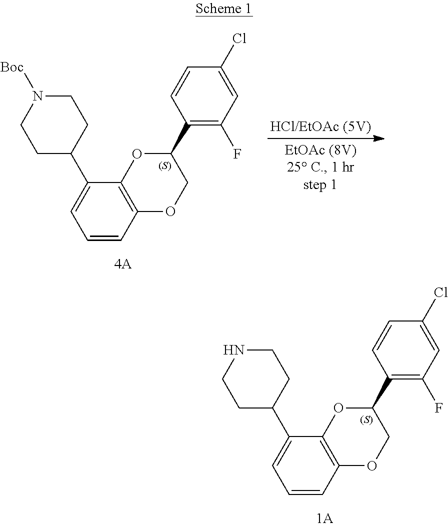

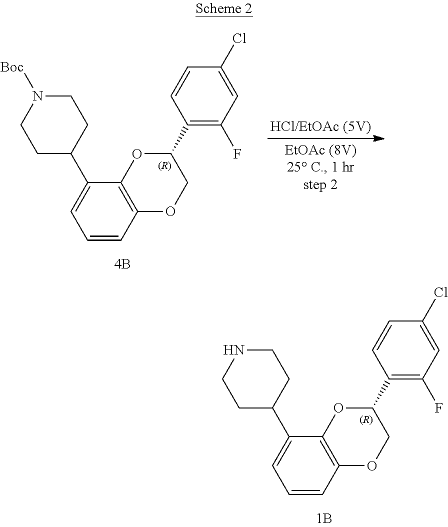

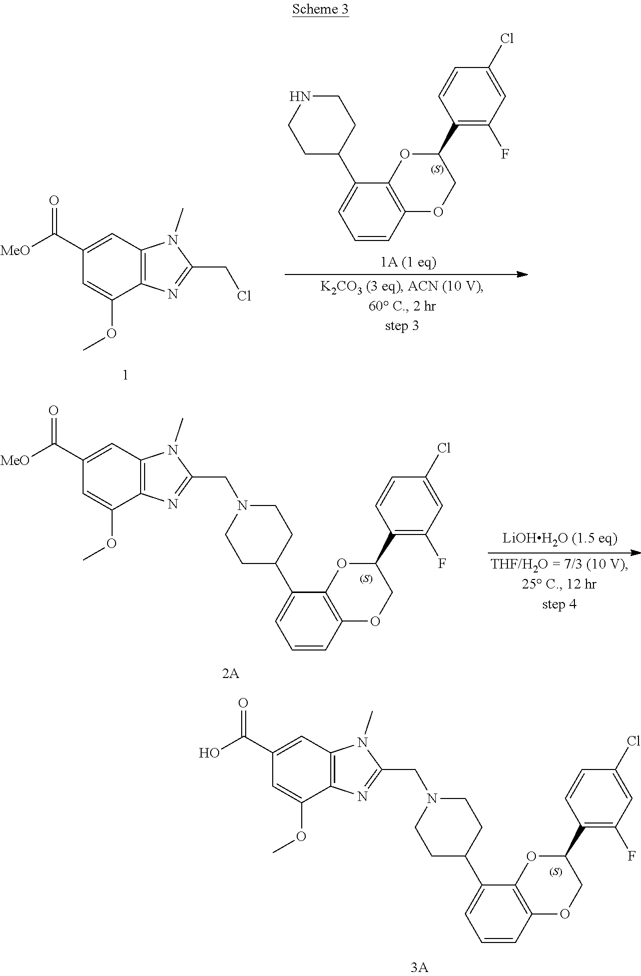

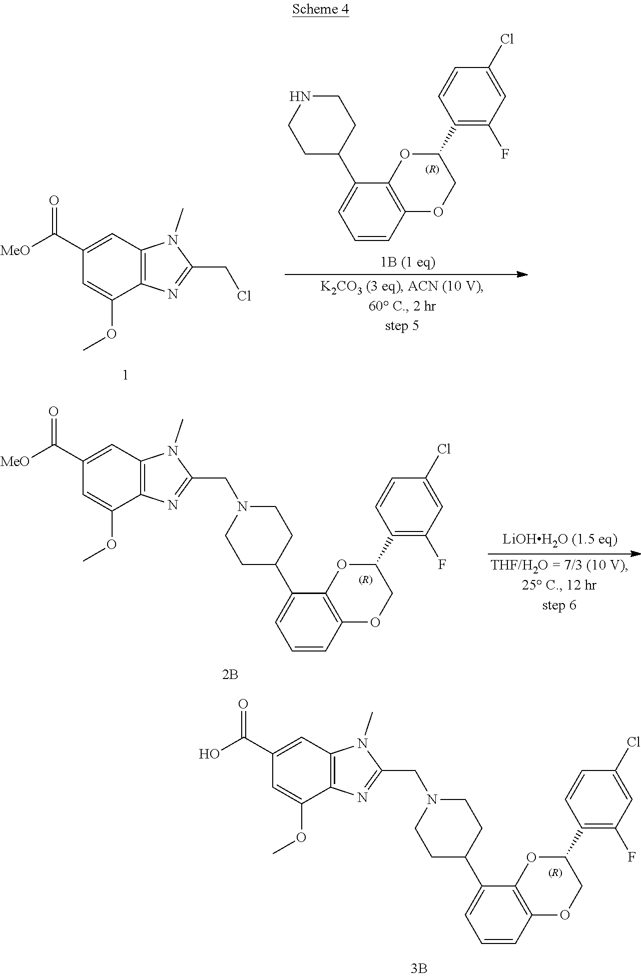

### General Procedure for Preparation of Intermediate 1A:

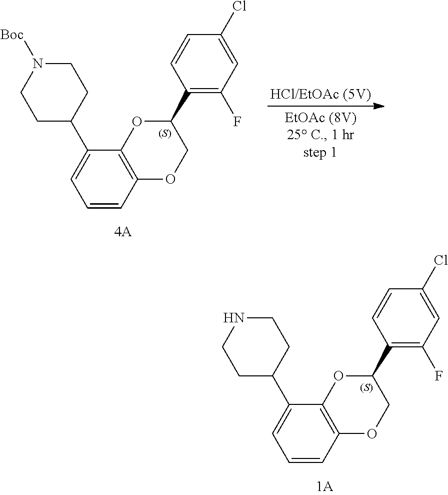

To a solution of 4A (80 mg, 178.60 μmol, 1 eq) in DCM (0.8 mL) was added HCl/EA (4M, 0.5 ml). The mixture was stirred at 20° C. for 1 hr. LC-MS showed 4A was consumed completely and one main peak with desired m/z was detected. The aqueous layer was neutralized by a.q. NaHCO₃ aq. at 0 C to pH around 7-8. The reaction mixture was diluted with H₂O 1 mL, extracted by DCM (2 mL*3). Then organic phase was washed by brine (2 mL*2). The organic was dried with Na₂SO₄, filtered. The organic phase was concentrated under reduced pressure at 45° C. to give a residue. 1A (60 mg, 172.51 μmol, 97% yield) was obtained as a white solid.

LCMS: RT=0.423 min, MS cal.: 347.1, 349.1, [M+H]⁻=348.1, 350.0

### General Procedure for Preparation of Intermediate 1b:

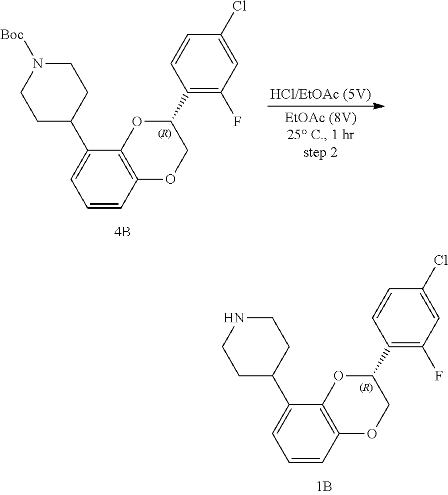

To a solution of 4B (320.00 mg, 714.40 μmol, 1 eq) in HCl/EA (4M, 3.2 mL) was stirred at 20° C. for 2 hr. TLC (PE/EA=5/1, Rf=0.00) indicated 4B was consumed completely and one new spot formed. The reaction was clean according to TLC. The reaction mixture was added a.q. Na₂CO₃ to adjust pH=10, diluted with H₂O 10 mL and extracted with EA 30 mL (10 mL*3). The combined organic layers were washed with brine 10 mL, dried over Na₂SO₄, filtered, and concentrated under reduced pressure to give a residue. 1B (230 mg, 661.28 μmol, 93% yield) was obtained as a white solid.

¹H NMR (400 MHz, DMSO-d6) δ=7.56 (dd, J=2.0, 10.4 Hz, 1H), 7.54-7.48 (m, 1H), 7.41 (dd, J=2.0, 8.4 Hz, 1H), 6.87-6.74 (m, 3H), 5.46 (dd, J=2.4, 7.6 Hz, 1H), 4.43 (dd, J=2.4, 11.6 Hz, 1H), 4.10 (dd, J=7.6, 11.2 Hz, 1H), 3.05-2.95 (m, 2H), 2.95-2.86 (m, 1H), 2.59-2.51 (m, 2H), 1.70 (d, J=12.0 Hz, 1H), 1.66-1.58 (m, 1H), 1.57-1.40 (m, 2H)

### General Procedure for Preparation of Intermediate 2A:

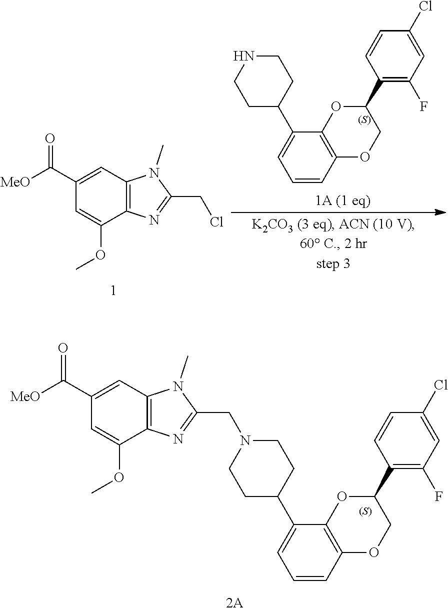

To a solution of 1 (65 mg, 186.88 μmol, 1 eq) in ACN (1 mL) was added K₂CO₃ (77.48 mg, 560.65 μmol, 3 eq) and 1A (50.21 mg, 186.88 μmol, 1 eq). The mixture was stirred at 60 C for 2 hr. TLC (PE/EA=0/1) indicated 1 was completely and one new spot formed. The reaction was clean according to TLC. The reaction mixture was diluted with H₂O (1 mL), extracted by EA (2 mL*3). Then organic phase was washed by brine (2 mL*2). The organic was dried with Na₂SO₄, filtered. The organic phase was concentrated under reduced pressure at 45° C. to give a residue. The residue was purified by column chromatography (SiO₂, PE/EA=100/0 to 0/1). 2A (100 mg, 155.16 μmol, 83.03% yield, 90% purity) was obtained as a white solid.

LCMS: RT=1.660 min, MS cal.: 579.2, 580.2, [M+H]⁻=580.1, 582.1

1H NMR (400 MHz, DMSO-d6) δ=7.81 (d, J=1.2 Hz, 1H), 7.57 (dd, J=2.0, 10.4 Hz, 1H), 7.54-7.49 (m, 1H), 7.42 (dd, J=2.0, 8.4 Hz, 1H), 7.26 (d, J=0.8 Hz, 1H), 6.85-6.75 (m, 3H), 5.46 (dd, J=2.0, 7.6 Hz, 1H), 4.44 (dd, J=2.0, 11.6 Hz, 1H), 4.10 (dd, J=8.0, 11.6 Hz, 1H), 3.97-3.97 (m, 1H), 3.96 (s, 3H), 3.88 (s, 5H), 3.81-3.76 (m, 2H), 2.87-2.86 (m, 1H), 2.96-2.86 (m, 1H), 2.86-2.76 (m, 1H), 2.22-2.08 (m, 2H), 1.82-1.53 (m, 4H)

### General Procedure for Preparation of Compound 268 (3A):

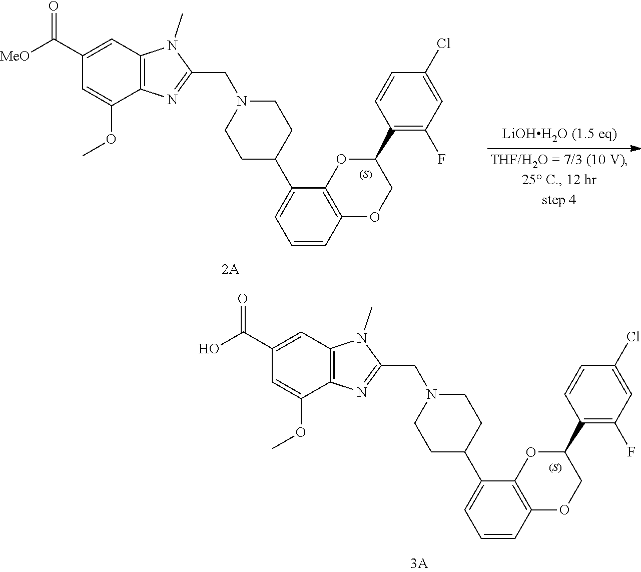

To a solution of 2A (80 mg, 137.92 μmol, 1 eq) in THF (0.7 mL) was added LiOH H₂O (8.68 mg, 206.88 μmol, 1.5 eq) and H₂O (0.3 mL). The mixture was stirred at 25° C. for 12 hr. LCMS showed 2A was consumed completely and one main peak with desired m/z was detected. The residue was purified by prep-HPLC (column: Waters Xbridge BEH C18 100*30 mm*10 um; mobile phase: [H₂O (10 mM NH₄HCO₃)-ACN]; gradient: 15%-65% B over 8.0 min). (S)-2-((4-(3-(4-Chloro-2-fluorophenyl)-2,3-dihydrobenzo[b][1,4]dioxin-5-yl)piperidin-1-yl)methyl)-4-methoxy-1-methyl-1H-benzo[d]imidazole-6-carboxylic acid (29.28 mg, 51.21 μmol, 37.13% yield, 99% purity) was obtained as a white solid.

LCMS: RT=2.741 min, MS cal.: 565.2, 566.2, [M+H]⁻=566.2, 568.2

HPLC: RT=11.484 min, purity=99.78%

1H NMR (400 MHz, DMSO-d6) δ=7.72 (d, J=1.2 Hz, 1H), 7.60-7.50 (m, 2H), 7.49-7.36 (m, 1H), 7.36-7.18 (m, 1H), 6.85-6.75 (m, 3H), 5.46 (dd, J=2.0, 8.0 Hz, 1H), 4.44 (dd, J=2.4, 11.6 Hz, 1H), 4.10 (dd, J=8.0, 11.6 Hz, 1H), 3.93 (s, 3H), 3.84 (s, 3H), 3.79-3.70 (m, 2H), 2.96-2.78 (m, 3H), 2.20-2.08 (m, 2H), 1.77 (br d, J=10.8 Hz, 1H), 1.81-1.53 (m, 4H)

### General Procedure for Preparation of Intermediate 2B:

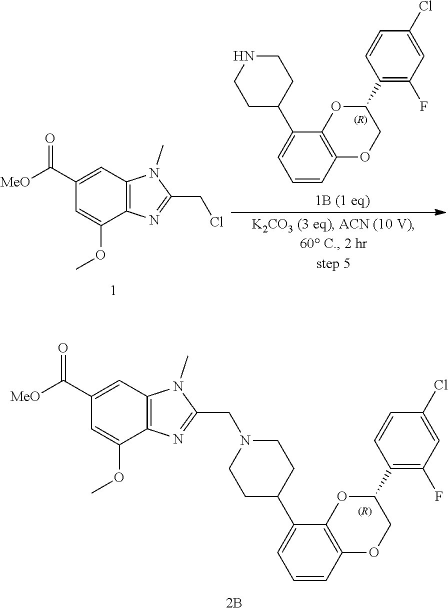

To a solution of 1 (50 mg, 186.08 μmol, 1 eq) in ACN (0.5 mL) was added K₂CO₃ (77.15 mg, 558.25 μmol, 3 eq) and 1B (64.72 mg, 186.08 μmol, 1 eq). The mixture was stirred at 60° C. for 2 hr. TLC (PE/EA=0/1, product Rf=0.30) indicated 1 was consumed completely and one new spot formed. The reaction was clean according to TLC. The reaction mixture was diluted with H₂O 5 mL and extracted with EA 15 mL (5 mL*3). The combined organic layers were washed with brine 5 mL, dried over Na₂SO₄, filtered, and concentrated under reduced pressure to give a residue. The residue was purified by column chromatography (SiO₂, PE/EA=10/1 to 1/4). 2B (90 mg, 155.16 μmol, 83% yield) was obtained as a white solid.

LCMS: RT=1.665 min, MS cal.: 579.19, 580.20, [M+H]⁻=580.1, 582.0

¹H NMR (400 MHz, DMSO-d6) δ=7.81 (d, J=0.8 Hz, 1H), 7.57 (dd, J=2.0, 10.4 Hz, 1H), 7.54-7.48 (m, 1H), 7.46-7.39 (m, 1H), 7.26 (s, 1H), 6.88-6.75 (m, 3H), 5.49-5.43 (m, 1H), 4.48-4.40 (m, 1H), 4.15-4.05 (m, 1H), 3.96 (s, 3H), 3.88 (s, 6H), 3.83-3.74 (m, 2H), 2.95-2.87 (m, 2H), 2.86-2.78 (m, 1H), 2.21-2.10 (m, 2H), 1.82-1.73 (m, 1H), 1.68 (s, 2H), 1.64-1.53 (m, 1H)

### General Procedure for Preparation of Compound 267 (3B):

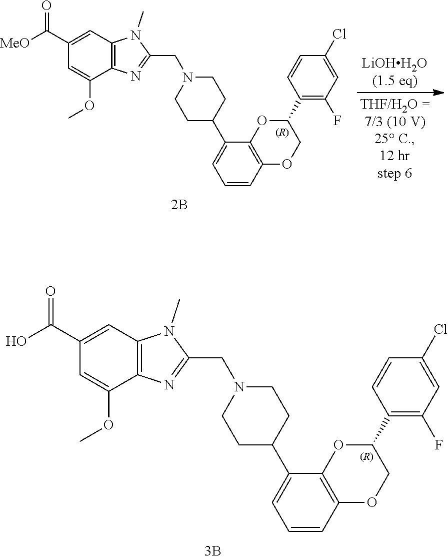

To a solution of 2B (90 mg, 155.16 μmol, 1 eq) in THF (0.7 mL) was added LiOH H₂O (9.77 mg, 232.74 μmol, 1.5 eq) in H₂O (0.3 mL). The mixture was stirred at 25° C. for 12 hr. LC-MS showed 2B was consumed completely and one main peak with desired mass was detected. The mixture was purified by prep-HPLC (neutral condition, column: Waters Xbridge BEH C18 100*30 mm*10 um; mobile phase: [H₂O (10 mM NH₄HCO₃)-ACN]; gradient: 10%-65% B over 8.0 min). (R)-2-((4-(3-(4-Chloro-2-fluorophenyl)-2,3-dihydrobenzo[b][1,4]dioxin-5-yl)piperidin-1-yl)methyl)-4-methoxy-1-methyl-1H-benzo[d]imidazole-6-carboxylic acid (28.81 mg, 50.72 μmol, 32.69% yield, 99.64% purity) was obtained as a white solid.

LCMS: RT=2.270 min, MS cal.: 565.2, 566.2, [M+H]⁻=566.1, 568.1

HPLC: Rt=11.517 min, purity=99.64%

¹H NMR (400 MHz, DMSO-d6) δ=7.72 (d, J=0.8 Hz, 1H), 7.57 (dd, J=2.0, 10.4 Hz, 1H), 7.55-7.49 (m, 1H), 7.46-7.40 (m, 1H), 7.27 (d, J=0.8 Hz, 1H), 6.85-6.75 (m, 3H), 5.46 (dd, J=2.0, 7.6 Hz, 1H), 4.44 (dd, J=2.4, 11.4 Hz, 1H), 4.10 (dd, J=8.0, 11.4 Hz, 1H), 3.93 (s, 3H), 3.85 (s, 3H), 3.81-3.70 (m, 2H), 2.91 (d, J=8.8 Hz, 2H), 2.87-2.78 (m, 1H), 2.19-2.09 (m, 2H), 1.69 (s, 2H), 1.64-1.54 (m, 1H), 1.77 (d, J=10.8 Hz, 1H)

### Example A247—Preparation of Compound 266. (S)-2-((4-(3-(4-chloro-2-fluorophenyl)-2,3-dihydrobenzo[b][1,4]dioxin-5-yl)piperidin-1-yl)methyl)-4-ethoxy-1-methyl-1H-benzo[d]imidazole-6-carboxylic acid (Compound 266)

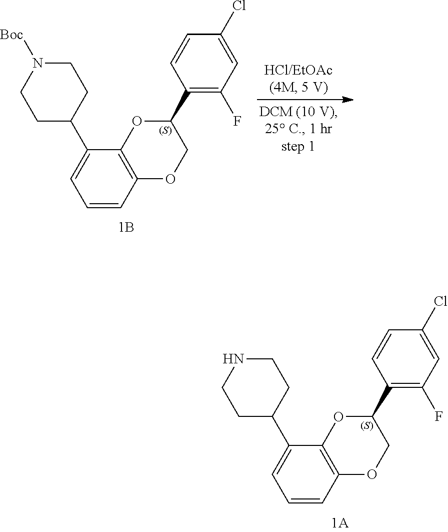

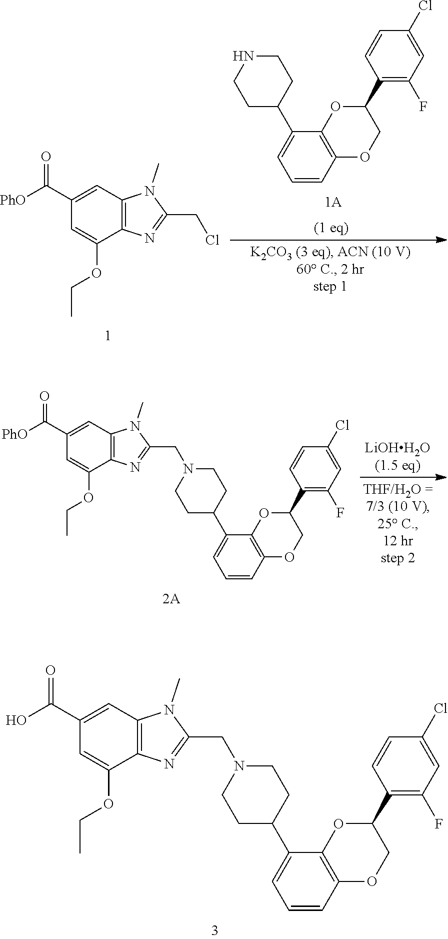 [SMILES: CCOc1cc(C(=O)Oc2ccccc2)cc2c1nc(CCl)n2C ; Fc1cc(Cl)ccc1[C@H]1COc2cccc(C3CCNCC3)c2O1 ; CCOc1cc(C(=O)Oc2ccccc2)cc2c1nc(CN1CCC(c3cccc4c3O[C@@H](c3ccc(Cl)cc3F)CO4)CC1)n2C ; CCOc1cc(C(=O)O)cc2c1nc(CN1CCC(c3cccc4c3O[C@@H](c3ccc(Cl)cc3F)CO4)CC1)n2C]

### General Procedure for Preparation of Compound 1A:

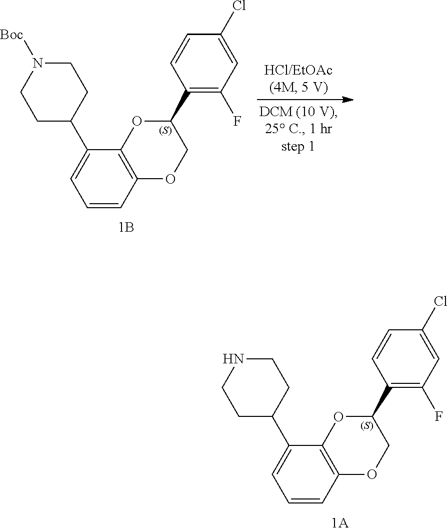

To a solution of compound 1B (240.00 mg, 535.80 μmol, 1 eq) in DCM (3 mL) was added HCl/EtOAc (4 M, 1 mL). The mixture was stirred at 25° C. for 1 hr. LC-MS (RT=0.426 min) showed compound 1B was consumed completely and one main peak with desired mass was detected. The reaction was poured into H₂O (10 ml). Then the reaction mixture was quenched with saturated aq. NaHCO₃10 mL to adjust pH=8-9. The mixture was extracted by DCM (20 mL*3). Then organic phase was combined and washed by H₂O (10 mL), brine (10 mL), dried over Na₂SO₄, filtered. The organic phase was concentrated under reduced pressure at 40° C. to give a product without further purification. Compound 1A (100 mg, 223.25 μmol, 41% yield) was obtained as a white solid.

LCMS: RT=0.457 min, MS cal.: 347.11/349.11, [M+H]⁺=348.10/350.10

### General Procedure for Preparation of Compound 2A:

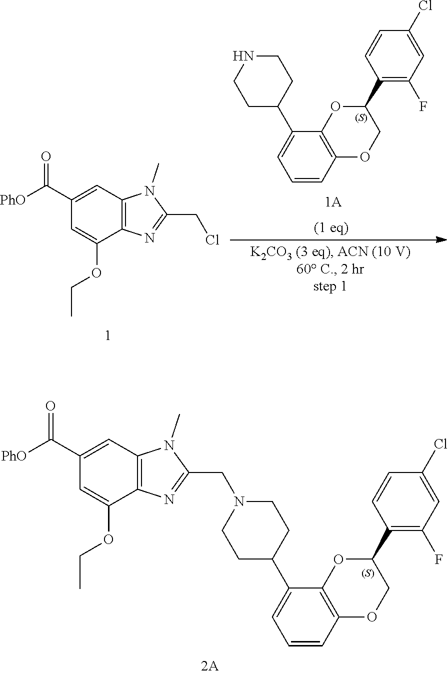

To a solution of compound 1 (95 mg, 275.53 μmol, 1 eq) in ACN (1 mL) was added K₂CO₃ (114.24 mg, 826.59 μmol, 3 eq) and compound 1A (95.83 mg, 275.53 μmol, 1 eq). The mixture was stirred at 60° C. for 2 hr. LC-MS (RT=0.559 min) showed compound 1 was consumed completely and one main peak with desired mass was detected. The reaction was poured into H₂O (10 ml). The mixture was extracted by EtOAc (10 mL*3). Then organic phase was combined and washed by H₂O (10 mL), brine (10 mL), dried with Na₂SO₄, filtered. The organic phase was concentrated under reduced pressure at 45° C. to give a residue. The residue was purified by column chromatography (SiO₂, PE/EtOAc=10/1 to 0/1). Compound 2A (120 mg, 182.89 μmol, 66% yield) was obtained as a white solid.

LCMS: RT=0.559 min, MS cal.: 655.22/656.23, [M+H]⁺=656.30/657.30

### General Procedure for Preparation of Compound 3:

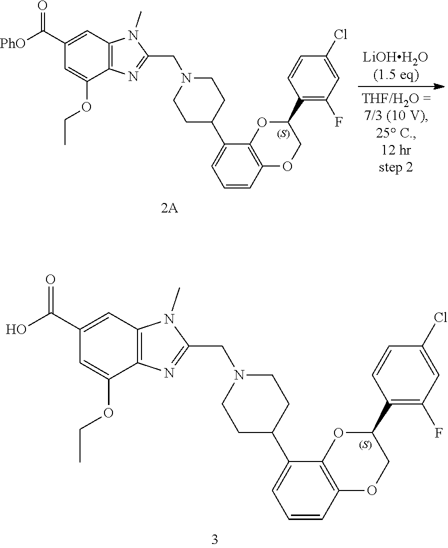

To a solution of compound 2A (110 mg, 167.65 μmol, 1 eq) in THF (0.7 mL) and H₂O (0.3 mL) was added LiOH·H₂O (10.55 mg, 251.47 μmol, 1.5 eq). The mixture was stirred at 25° C. for 12 hr. LC-MS (RT=1.205 min) showed compound 2A was consumed completely and one main peak with desired mass was detected. The reaction was filtered directly, and the filtrate was purified by prep-HPLC (column: 2_Phenomenex Gemini C18 75*40 mm*3 um; mobile phase: [H₂O (10 mM NH₄HCO₃)-ACN]; gradient: 30%-60% B over 8.0 min). (S)-2-((4-(3-(4-chloro-2-fluorophenyl)-2,3-dihydrobenzo[b][1,4]dioxin-5-yl)piperidin-1-yl)methyl)-4-ethoxy-1-methyl-1H-benzo[d]imidazole-6-carboxylic acid (22.28 mg, 38.41 μmol, 22% yield) was obtained as a white solid.

HPLC: RT=12.076 min, purity: 99.74%

LCMS: RT=2.815 min, MS cal.: 579.19/580.20, [M+H]⁺=580.20/581.30

¹H NMR (400 MHz, DMSO-d6) δ=7.71 (s, 1H), 7.57 (dd, J=2.0, 10.4 Hz, 1H), 7.52 (t, J=8.0 Hz, 1H), 7.45-7.41 (m, 1H), 7.25 (s, 1H), 6.85-6.76 (m, 3H), 5.46 (dd, J=2.0, 8.0 Hz, 1H), 4.48-4.40 (m, 1H), 4.29-4.20 (m, 2H), 4.10 (dd, J=8.0, 11.6 Hz, 1H), 3.85 (s, 3H), 3.81-3.72 (m, 2H), 2.95-2.79 (m, 3H), 2.20-2.09 (m, 2H), 1.76 (br s, 1H), 1.69 (br s, 2H), 1.65-1.53 (m, 1H), 1.41 (t, J=7.2 Hz, 3H).

### Example A248—Preparation of Compound 265. (R)-2-((4-(3-(4-chloro-2-fluorophenyl)-2,3-dihydrobenzo[b][1,4]dioxin-5-yl)piperidin-1-yl)methyl)-4-ethoxy-1-methyl-1H-benzo[d]imidazole-6-carboxylic acid (Compound 265) (3)

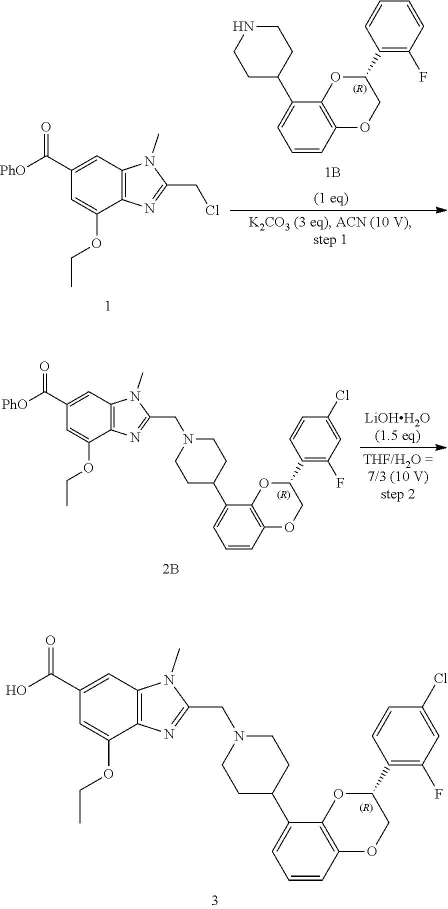

### General Procedure for Preparation of Intermediate 2B:

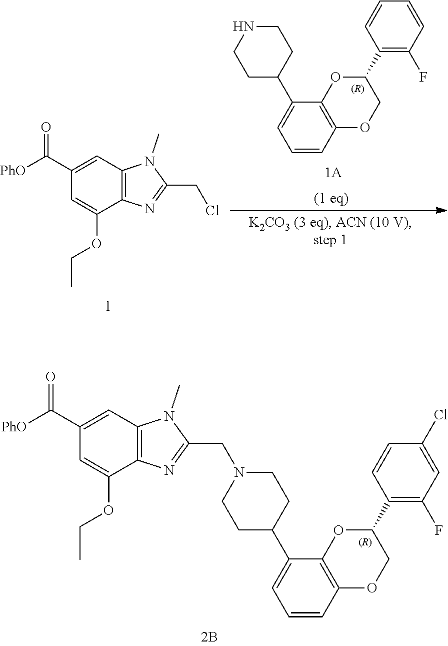

A mixture of 1 (100 mg, 290.03 μmol, 1 eq), 1B (100.88 mg, 290.03 μmol, 1 eq), K₂CO₃ (120.25 mg, 870.09 μmol, 3 eq), in CH₃CN (1 mL) was degassed and purged with N₂ for 3 times, and then the mixture was stirred at 60° C. for 2 hr under N₂ atmosphere. LC-MS (RT=1.867 min) showed 1 was consumed completely and one main peak with desired mass was detected. The reaction mixture was diluted with H₂O 10 mL and extracted with EtOAc 30 mL (10 mL*3). The combined organic layers were washed with brine 10 mL, dried over Na₂SO₄, filtered, and concentrated under reduced pressure to give a residue. The residue was purified by column chromatography (SiO₂, Petroleum ether/Ethyl acetate=100/1 to 1/1). Compound 2B (100 mg, 152.41 μmol, 52.55% yield) was obtained as a white solid.

LCMS: Rt₁=1.867 min, MS cal.: 655.2, [M+H]⁺=656.1

1H NMR (400 MHz, DMSO-d6) δ=8.01 (s, 1H) 7.60-7.55 (m, 1H) 7.54-7.46 (m, 3H) 7.43 (d, J=8.0 Hz, 1H) 7.38 (s, 1H) 7.35-7.26 (m, 3H) 6.85-6.74 (m, 3H) 5.46 (d, J=6.8 Hz, 1H) 4.44 (d, J=9.8 Hz, 1H) 4.29 (q, J=6.8 Hz, 2H) 4.10 (dd, J=11.2, 8.11 Hz, 1H) 3.92 (s, 3H) 3.82 (s, 2H) 2.92 (d, J=10.8 Hz, 2H) 2.87-2.78 (m, 1H) 2.24-2.12 (m, 2H) 1.78 (d, J=11.2 Hz, 1H) 1.70 (s, 2H) 1.64-1.55 (m, 1H) 1.43 (t, J=6.8 Hz, 3H)

### General Procedure for Preparation of Compound 265 (3):

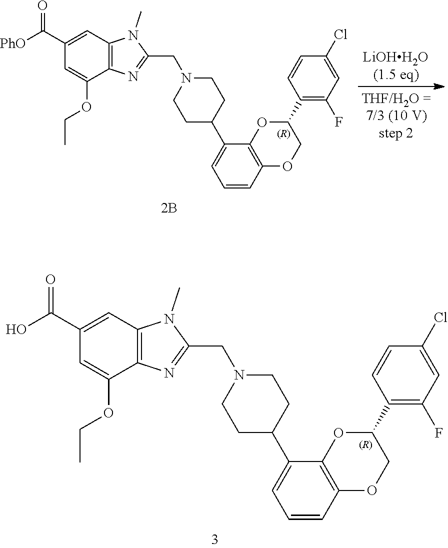

A mixture of 2B (100.00 mg, 152.41 μmol, 1 eq), LiOH·H₂O (9.59 mg, 228.61 μmol, 1.5 eq), in THF (0.7 mL) H₂O (0.3 mL) was degassed and purged with N₂ for 3 times, and then the mixture was stirred at 25° C. for 16 hr under N₂ atmosphere. LC-MS (ET43536-1764-P1A1 RT=1.202 MIN) showed 2B was consumed completely and one main peak with desired mass was detected. The reaction mixture was purified by prep-HPLC (neutral condition column: Waters Xbridge Prep OBD C18 150*40 mm*10 um; mobile phase: [H₂O (10 mM NH₄HCO₃)-ACN]; gradient: 30%-80% B over 8.0 min). (R)-2-((4-(3-(4-chloro-2-fluorophenyl)-2,3-dihydrobenzo[b][1,4]dioxin-5-yl)piperidin-1-yl)methyl)-4-ethoxy-1-methyl-1H-benzo[d]imidazole-6-carboxylic acid (15.21 mg, 25.52 μmol, 16.74% yield) was obtained as a white solid.

LCMS: Rt₁=2.414 min, MS cal.: 579.2, [M+H]⁺=580.2

1H NMR (400 MHz, METHANOL-d4) δ=7.77 (d, J=1.2 Hz, 1H) 7.51 (t, J=7.6 Hz, 1H) 7.42 (d, J=1.2 Hz, 1H) 7.31 (d, J=8.4 Hz, 2H) 6.83-6.79 (m, 2H) 6.77-6.71 (m, 1H) 5.40 (dd, J=8.4, 2.15 Hz, 1H) 4.40 (dd, J=11.2, 2.32 Hz, 1H) 4.27 (q, J=6.8 Hz, 2H) 4.01 (dd, J=11.2, 8.11 Hz, 1H) 3.92 (s, 3H) 3.85 (s, 2H) 3.03-2.90 (m, 3H) 2.35-2.19 (m, 2H) 1.90-1.72 (m, 4H) 1.51 (t, J=6.8 Hz, 3H)

### Example A249—Preparation of Compound 262. rel-(S)-2-((4-(6-((4-Chloro-2-fluorobenzyl)oxy)pyridin-2-yl)piperidin-1-yl)methyl)-4-((1-fluoropropan-2-yl)oxy)-1-methyl-1H-benzo[d]imidazole-6-carboxylic acid (Compound 263) (3A) & rel-(R)-2-((4-(6-((4-Chloro-2-fluorobenzyl)oxy)pyridin-2-yl)piperidin-1-yl)methyl)-4-((1-fluoropropan-2-yl)oxy)-1-methyl-1H-benzo[d]imidazole-6-carboxylic acid (Compound 262) (3B)

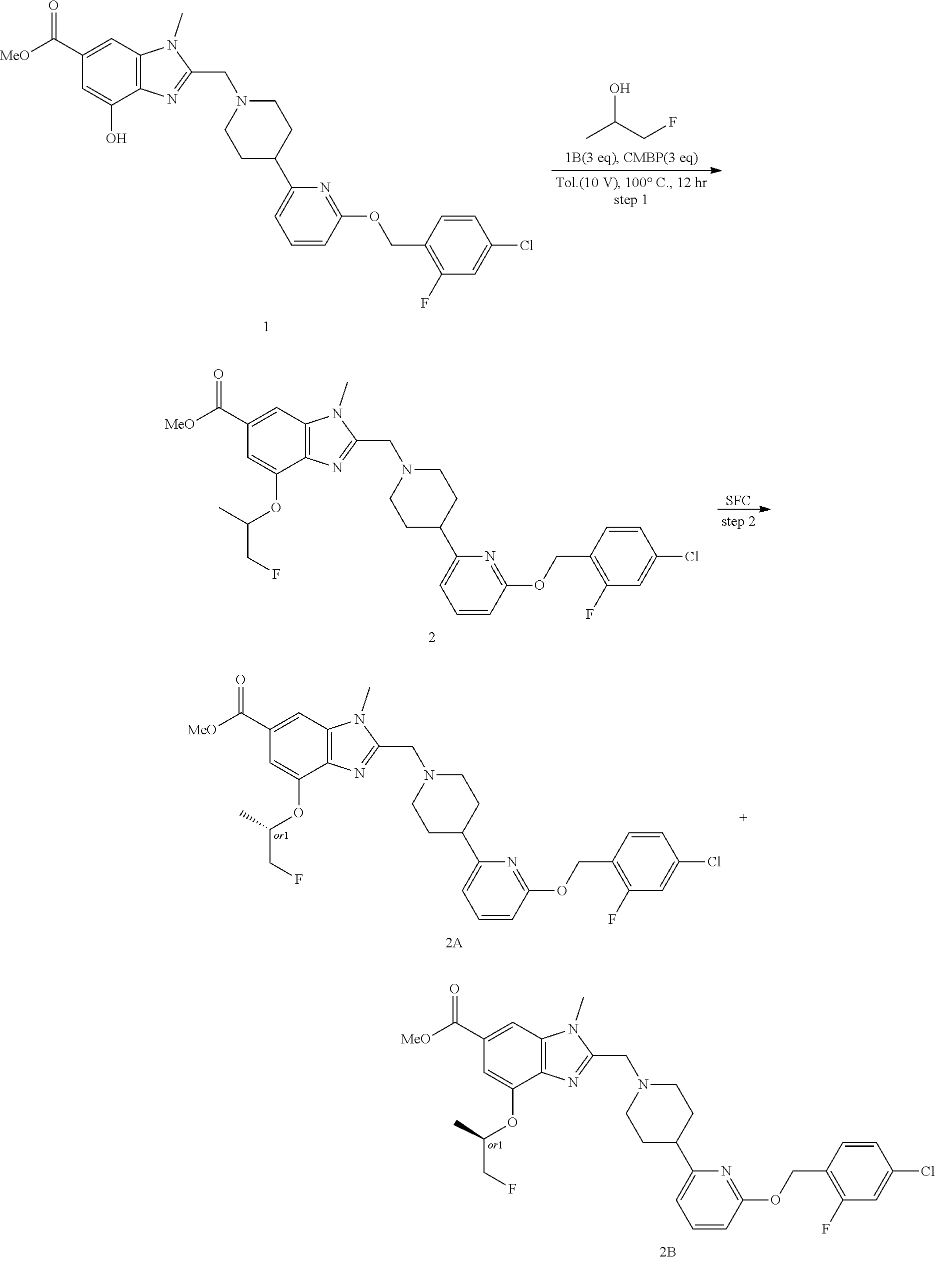

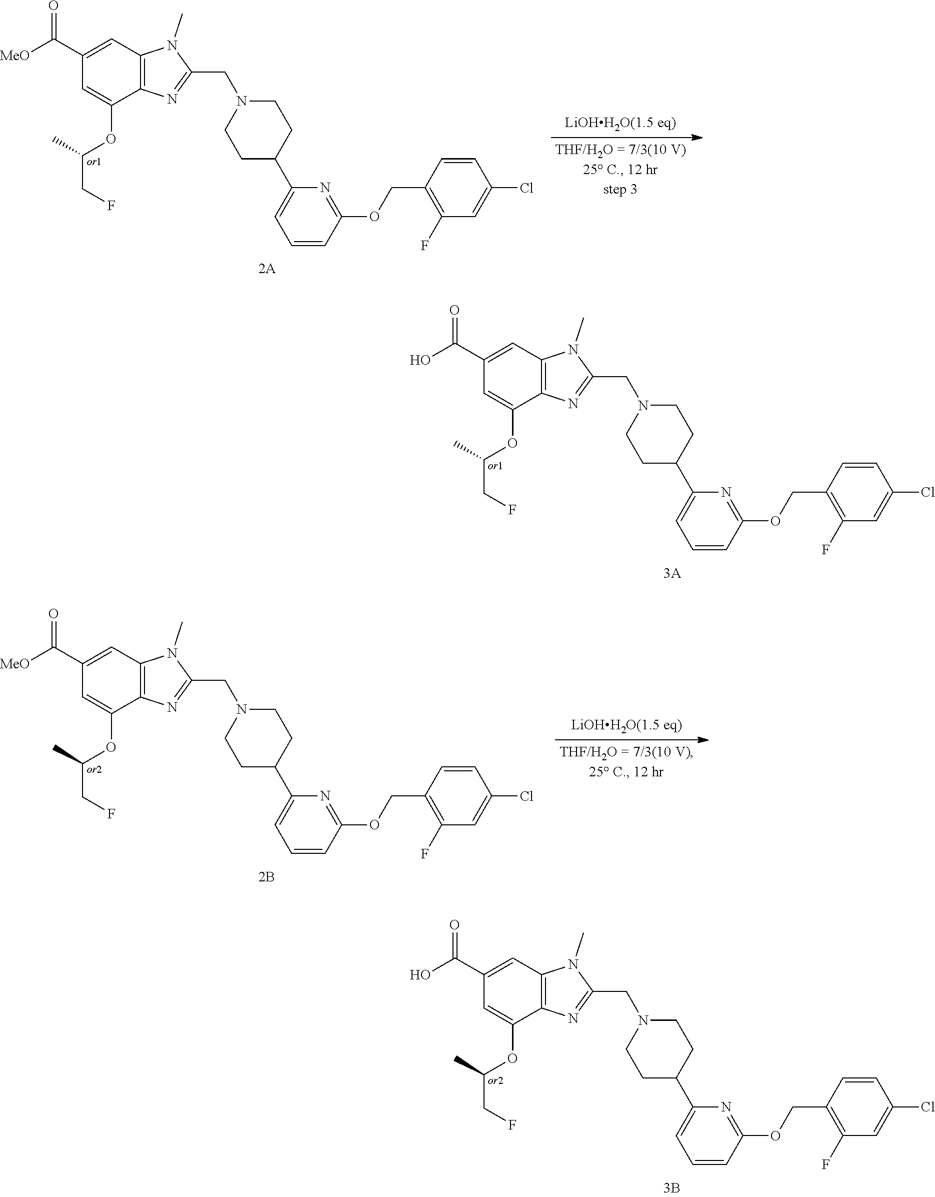 [SMILES: COC(=O)c1cc(O[C@@H](C)CF)c2nc(CN3CCC(c4cccc(OCc5ccc(Cl)cc5F)n4)CC3)n(C)c2c1 ; C[C@@H](CF)Oc1cc(C(=O)O)cc2c1nc(CN1CCC(c3cccc(OCc4ccc(Cl)cc4F)n3)CC1)n2C ; COC(=O)c1cc(O[C@H](C)CF)c2nc(CN3CCC(c4cccc(OCc5ccc(Cl)cc5F)n4)CC3)n(C)c2c1 ; C[C@H](CF)Oc1cc(C(=O)O)cc2c1nc(CN1CCC(c3cccc(OCc4ccc(Cl)cc4F)n3)CC1)n2C]

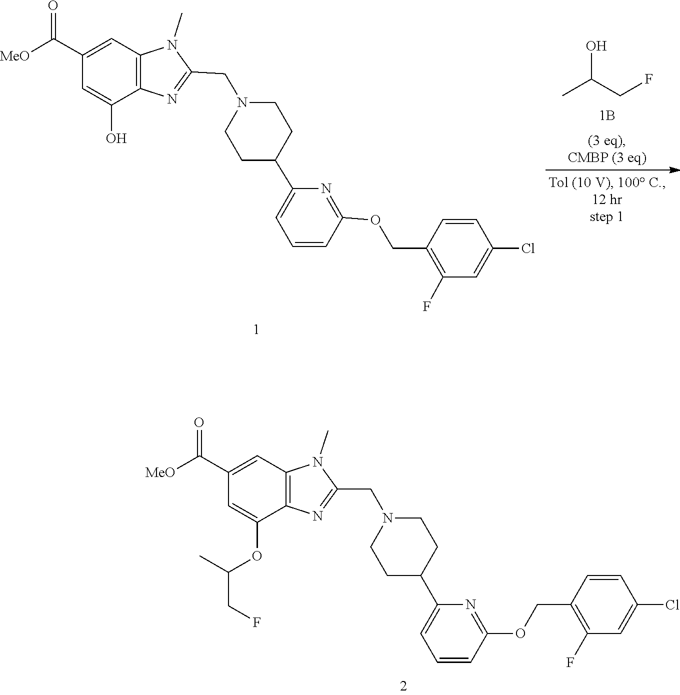

### General Procedure for Preparation of Intermediate 2:

To a solution of 1 (500 mg, 927.65 μmol, 1 eq) in toluene (5 mL) was added 1B (217.31 mg, 2.78 mmol, 3 eq) and CMBP (671.67 mg, 2.78 mmol, 3 eq). The mixture was stirred at 100° C. for 12 hr. LCMS showed 1 was consumed completely and one main peak with desired m/z was detected. TLC (Petroleum ether:Ethyl acetate=0:1) indicated 1 was consumed completely and one new spot formed. The reaction was clean according to TLC. After 12 hr, the reaction mixture was diluted with H₂O (10 mL), extracted by Ethyl acetate 15 mL (5 mL*3). Then organic phase was combined and washed by brine 10 mL, dried with Na₂SO₄, filtered. The organic phase was concentrated under reduced pressure at 40° C. to give a residue. The residue was purified by column chromatography (SiO₂, Petroleum ether/Ethyl acetate=20/1 to 0/1). 2 (350 mg, 560.87 μmol, 60.46% yield, 96% purity) was obtained as a white solid.

LCMS: RT=1.706 min, MS cal.: 598.22, 599.22, [M+H]⁻=599.1

1H NMR (400 MHz, DMSO-d6) δ=7.84-7.81 (m, 1H), 7.62 (t, J=7.6 Hz, 1H), 7.58-7.51 (m, 1H), 7.45 (dd, J=2.0, 10.0 Hz, 1H), 7.32-7.30 (m, 1H), 7.30-7.25 (m, 1H), 6.86 (d, J=7.2 Hz, 1H), 6.66 (d, J=8.4 Hz, 1H), 5.35 (s, 2H), 5.33-5.22 (m, 1H), 4.75-4.49 (m, 2H), 3.92 (s, 3H), 3.87 (s, 3H), 3.84-3.79 (m, 2H), 2.92 (d, J=11.2 Hz, 2H), 2.63-2.53 (m, 1H), 2.52-2.48 (m, 4H), 2.20 (t, J=10.8 Hz, 2H), 1.82-1.63 (m, 4H), 1.31 (d, J=5.6 Hz, 3H)

### General Procedure for Preparation of Intermediate 2A & 2B:

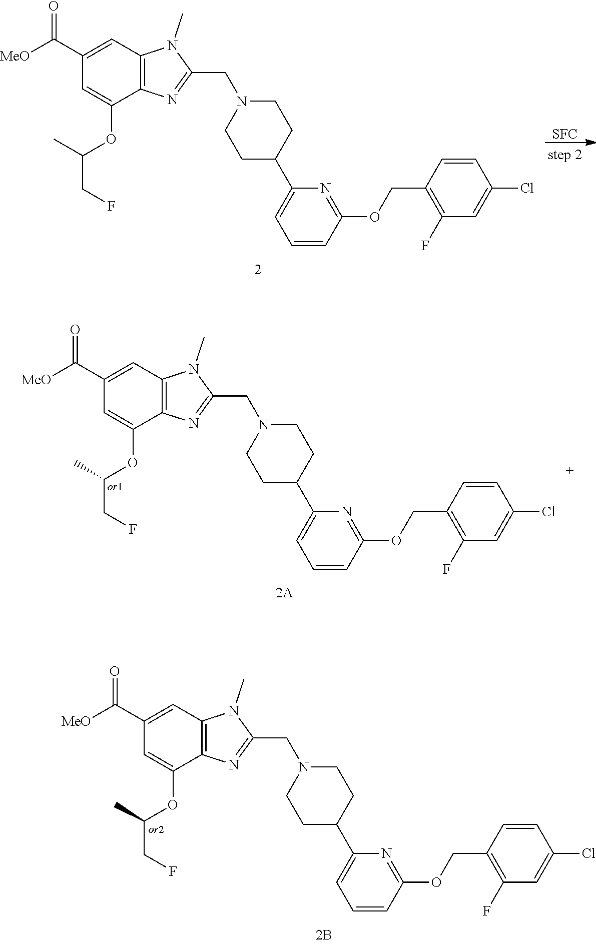 [SMILES: COC(=O)c1cc(OC(C)CF)c2nc(CN3CCC(c4cccc(OCc5ccc(Cl)cc5F)n4)CC3)n(C)c2c1 ; "SFC" ; COC(=O)c1cc(O[C@@H](C)CF)c2nc(CN3CCC(c4cccc(OCc5ccc(Cl)cc5F)n4)CC3)n(C)c2c1 ; COC(=O)c1cc(O[C@H](C)CF)c2nc(CN3CCC(c4cccc(OCc5ccc(Cl)cc5F)n4)CC3)n(C)c2c1]

To a solution of 2 (350 mg, 584.24 μmol, 1 eq) was special separated by SFC (column: DAICEL CHIRALCEL OX (250 mm*30 mm, 10 um) mobile phase: [CO₂-IPA (0.1% NH₃H₂O)] B %: 45%, isocratic elution mode). 2A (200 mg, 333.85 μmol, 57.14% yield) and 2B (160 mg, 267.08 μmol, 45.71% yield) was obtained as an orange oil.

LCMS (2A): Rt=1.723 min, MS cal.: 598.22, 599.22, [M+H]⁺=599.1

LCMS (2B): Rt=1.719 min, MS cal.: 598.22, 599.22, [M+H]⁺=599.1

1H NMR (2A) (400 MHz, DMSO-d6) δ=7.83 (d, J=1.2 Hz, 1H), 7.62 (t, J=8.0 Hz, 1H), 7.55 (t, J=8.4 Hz, 1H), 7.45 (dd, J=2.0, 10.0 Hz, 1H), 7.32-7.26 (m, 2H), 6.86 (d, J=7.2 Hz, 1H), 6.66 (d, J=8.4 Hz, 1H), 5.35 (s, 2H), 5.34-5.21 (m, 1H), 4.74-4.49 (m, 2H), 3.93 (s, 3H), 3.87 (s, 3H), 3.85-3.79 (m, 2H), 2.93 (d, J=11.2 Hz, 2H), 2.64-2.54 (m, 1H), 2.20 (t, J=10.8 Hz, 2H), 1.82-1.64 (m, 4H), 1.31 (dd, J=1.2, 6.4 Hz, 3H)

1H NMR (2B) (400 MHz, DMSO-d6) δ=7.83 (s, 1H), 7.62 (t, J=8. Hz, 1H), 7.55 (t, J=8.0 Hz, 1H), 7.45 (dd, J=2.0, 10.0 Hz, 1H), 7.34-7.24 (m, 2H), 6.86 (d, J=7.2 Hz, 1H), 6.66 (d, J=8.4 Hz, 1H), 5.36 (s, 2H), 4.74-4.49 (m, 2H), 3.93 (s, 3H), 3.87 (s, 3H), 3.82 (s, 2H), 3.63-3.57 (m, 1H), 2.93 (d, J=11.2 Hz, 2H), 2.65-2.54 (m, 1H), 2.20 (t, J=11.2 Hz, 2H), 1.84-1.63 (m, 5H), 1.31 (dd, J=0.8, 6.4 Hz, 3H)

### General Procedure for Preparation of Compound 263 (3A):

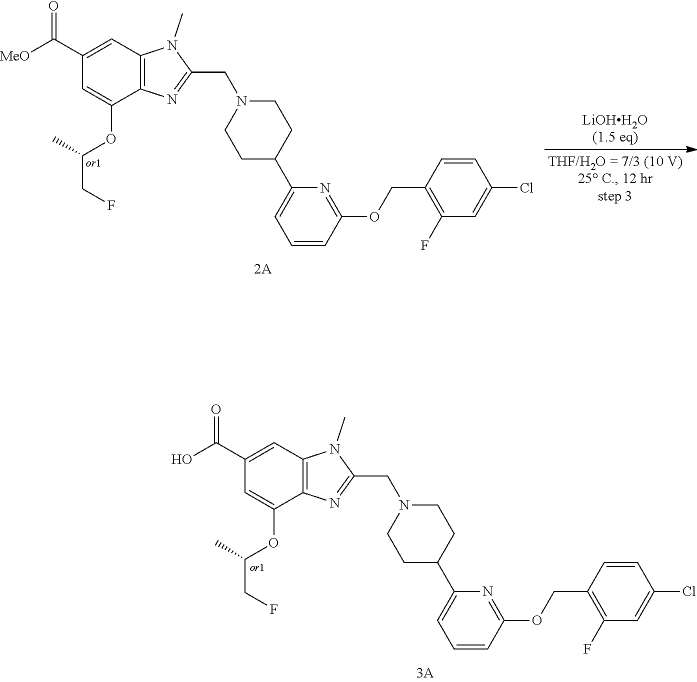 [SMILES: COC(=O)c1cc(O[C@@H](C)CF)c2nc(CN3CCC(c4cccc(OCc5ccc(Cl)cc5F)n4)CC3)n(C)c2c1 ; C[C@@H](CF)Oc1cc(C(=O)O)cc2c1nc(CN1CCC(c3cccc(OCc4ccc(Cl)cc4F)n3)CC1)n2C]

To a solution of 2A (200 mg, 333.85 μmol, 1 eq) in THF (1.4 mL) was added LiOH·H₂O (21.01 mg, 500.78 μmol, 1.5 eq) and H₂O (0.6 mL). The mixture was stirred at 25° C. for 12 hr. LCMS showed 2A was consumed completely and one main peak with desired m/z was detected. The residue was purified by prep-HPLC (column: Waters Xbridge Prep OBD C18 150*40 mm*10 um; mobile phase: [H₂O (10 mM NH₄HCO₃)-ACN]; gradient: 30%-60% B over 8.0 min). rel-(S)-2-((4-(6-((4-Chloro-2-fluorobenzyl)oxy)pyridin-2-yl)piperidin-1-yl)methyl)-4-((1-fluoropropan-2-yl)oxy)-1-methyl-1H-benzo[d]imidazole-6-carboxylic acid (28.54 mg, 48.30 μmol, 14.47% yield, 99% purity) was obtained as a white solid.

LCMS: Rt=2.799 min, MS cal.: 584.20, 585.20, [M+H]⁺=585.2

HPLC: Rt=12.058 min, purity=99.61%

¹H NMR (400 MHz, DMSO-d₄) δ=7.79 (s, 1H), 7.65-7.59 (m, 1H), 7.55 (t, J=8.0 Hz, 1H), 7.45 (dd, J=2.0, 10.0 Hz, 1H), 7.31-7.26 (m, 2H), 6.86 (d, J=7.2 Hz, 1H), 6.66 (d, J=8.0 Hz, 1H), 5.36 (s, 2H), 5.33-5.19 (m, 1H), 4.74-4.47 (m, 2H), 3.91 (s, 3H), 3.84 (s, 1H), 2.93 (d, J=11.2 Hz, 2H), 2.64-2.53 (m, 1H), 2.20 (t, J=10.8 Hz, 2H), 1.82-1.64 (m, 4H), 1.31 (dd, J=1.2, 6.4 Hz, 3H)

### General Procedure for Preparation of Compound 262 (3B):

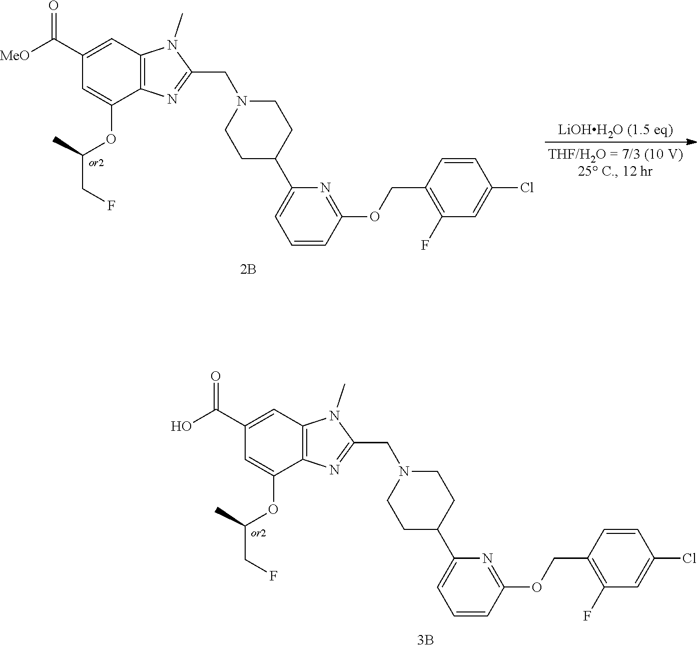 [SMILES: COC(=O)c1cc(O[C@H](C)CF)c2nc(CN3CCC(c4cccc(OCc5ccc(Cl)cc5F)n4)CC3)n(C)c2c1 ; C[C@H](CF)Oc1cc(C(=O)O)cc2c1nc(CN1CCC(c3cccc(OCc4ccc(Cl)cc4F)n3)CC1)n2C]

To a solution of 2B (140 mg, 233.70 μmol, 1 eq) in THF (0.98 mL) and H₂O (0.42 mL) was added LiOH·H₂O (1 M, 350.55 μL, 1.5 eq). The mixture was stirred at 25° C. for 12 hr. LC-MS showed 2B was consumed completely and desired mass was detected. The mixture was concentrated to give a crude product. The crude product was purified by reversed-phase HPLC (column: Waters Xbridge Prep OBD C18 150*40 mm*10 um; mobile phase: [H₂O (10 mM NH₄HCO₃)-ACN]; gradient: 20%-70% B over 8.0 min). rel-(R)-2-((4-(6-((4-Chloro-2-fluorobenzyl)oxy)pyridin-2-yl)piperidin-1-yl)methyl)-4-((1-fluoropropan-2-yl)oxy)-1-methyl-1H-benzo[d]imidazole-6-carboxylic acid (29.53 mg, 50.48 μmol, 21.60% yield) was obtained as a white solid.

LCMS: RT=2.786 min, MS cal.: 585.0, [M+H]⁺=585.2

HPLC: RT=12.017 min, purity: 99.6%

¹H NMR (400 MHz, DMSO-d6) δ=7.79 (d, J=1.2 Hz, 1H), 7.64-7.59 (m, 1H), 7.55 (t, J=8.0 Hz, 1H), 7.45 (dd, J=2.0, 10.0 Hz, 1H), 7.31-7.26 (m, 2H), 6.86 (d, J=7.6 Hz, 1H), 6.66 (d, J=8.0 Hz, 1H), 5.36 (s, 2H), 5.32-5.21 (m, 1H), 4.73-4.49 (m, 2H), 3.91 (s, 3H), 3.81 (s, 2H), 2.93 (br d, J=11.2 Hz, 2H), 2.62-2.55 (m, 1H), 2.24-2.15 (m, 2H), 1.83-1.62 (m, 4H), 1.31 (dd, J=1.2, 6.8 Hz, 3H)

### Example A250—Preparation of Compound 261. 2-((4-(6-((4-Cyano-2-fluorobenzyl)oxy)pyridin-2-yl)piperidin-1-yl)methyl)-4-ethoxy-1-methyl-N-(methylsulfonyl)-1H-benzo[d]imidazole-6-carboxamide (Compound 261) (4)

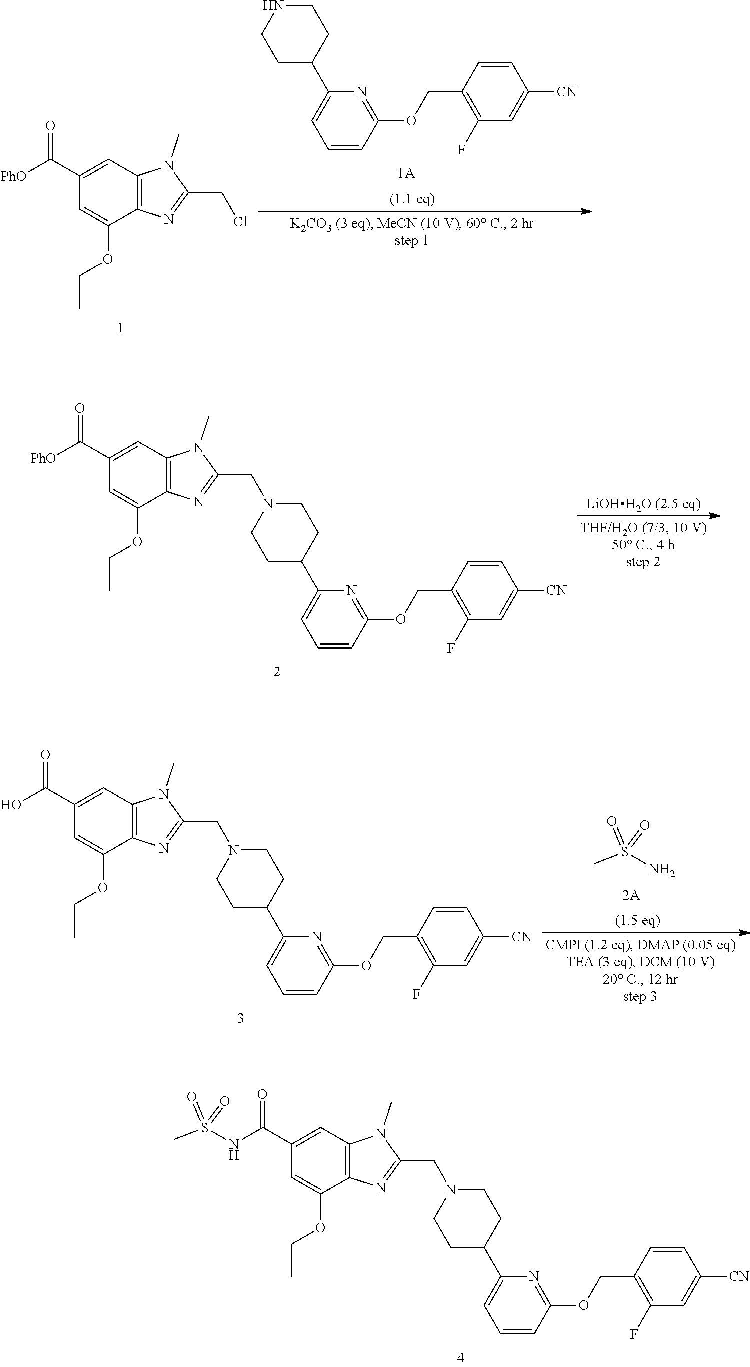

### General Procedure for Preparation of Intermediate 2:

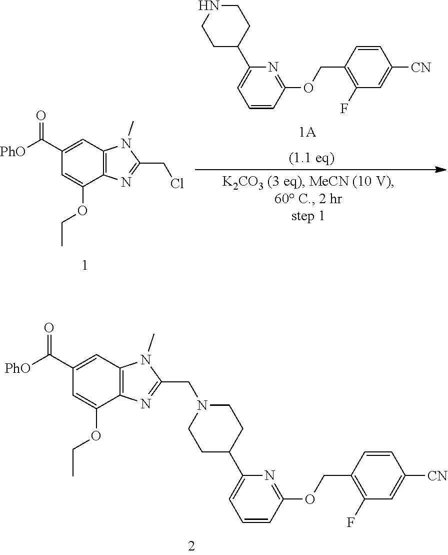 [SMILES: N#Cc1ccc(COc2cccc(C3CCNCC3)n2)c(F)c1 ; CCOc1cc(C(=O)Oc2ccccc2)cc2c1nc(CCl)n2C ; CCOc1cc(C(=O)Oc2ccccc2)cc2c1nc(CN1CCC(c3cccc(OCc4ccc(C#N)cc4F)n3)CC1)n2C]

To a solution of 1 (450 mg, 1.31 mmol, 1 eq) and 1A (588.33 mg, 1.44 mmol, 1.1 eq, 2.7HCl) in ACN (4.5 mL) was added K₂CO₃ (541.13 mg, 3.92 mmol, 3 eq) at 20° C. The mixture was stirred at 60° C. for 2 hr. LC-MS (Rt=0.504) showed 1 was consumed completely and desired mass was detected. The reaction mixture was diluted with H₂O (30 mL) and extracted with EtOAc (10 mL*3). The combined organic layers were washed with brine (20 mL*2), dried over Na₂SO₄, filtered and concentrated under reduced pressure to give a residue. The residue was purified by column chromatography (SiO₂, Petroleum ether/Ethyl acetate=10/1 to 1/1). 2 (740 mg, 1.19 mmol, 91% yield) was obtained as a pink solid.

LCMS: RT=0.504 min, MS cal.: 619.26, [M+H]⁺=620.4

¹H NMR (400 MHz, DMSO-d6) δ=8.02 (s, 1H), 7.88 (d, J=10.0 Hz, 1H), 7.69 (d, J=3.6 Hz, 2H), 7.64 (t, J=8.0 Hz, 1H), 7.49 (t, J=8.0 Hz, 2H), 7.38 (s, 1H), 7.35-7.25 (m, 3H), 6.87 (d, J=7.2 Hz, 1H), 6.71 (d, J=8.0 Hz, 1H), 5.45 (s, 2H), 4.31 (q, J=7.2 Hz, 2H), 3.96 (s, 3H), 3.84 (s, 2H), 2.91 (d, J=11.2 Hz, 2H), 2.63-2.53 (m, 1H), 2.19 (t, J=10.0 Hz, 2H), 1.81-1.59 (m, 4H), 1.43 (t, J=6.8 Hz, 3H)

### General Procedure for Preparation of Intermediate 3:

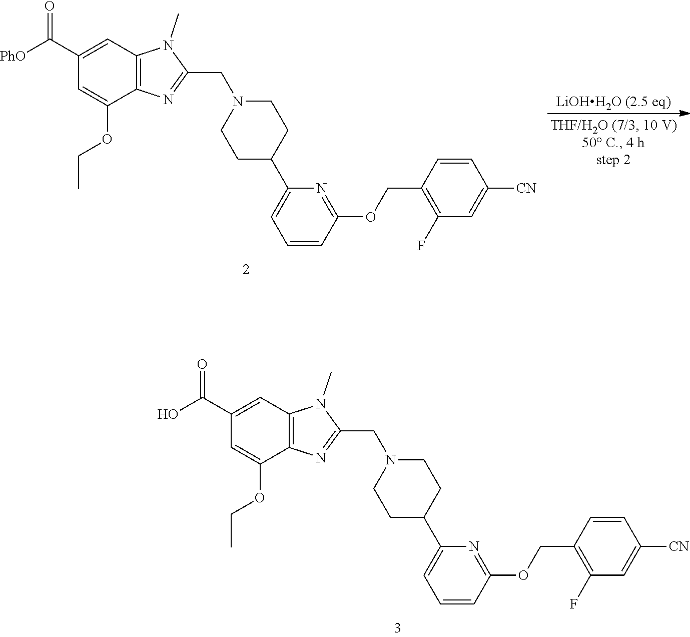 [SMILES: CCOc1cc(C(=O)Oc2ccccc2)cc2c1nc(CN1CCC(c3cccc(OCc4ccc(C#N)cc4F)n3)CC1)n2C ; CCOc1cc(C(=O)O)cc2c1nc(CN1CCC(c3cccc(OCc4ccc(C#N)cc4F)n3)CC1)n2C]

To a solution of 2 (400 mg, 645.49 μmol, 1 eq) in THF (2.8 mL) was added LiOH·H₂O (67.72 mg, 1.61 mmol, 2.5 eq) in H₂O (1.2 mL) at 20° C. The mixture was stirred at 50° C. for 4 hr. LC-MS (Rt=0.427) showed 2 was consumed completely and desired mass was detected. The reaction mixture was concentrated under reduced pressure to remove THF, diluted with H₂O (10 mL) and adjusted pH to 3-4, which was extracted with EtOAc (5 mL*3). The combined organic layers were washed with brine (5 mL*2), dried over Na₂SO₄, filtered, and concentrated under reduced pressure to give a crude product. The residue was purified by column chromatography (SiO₂, Petroleum ether/Ethyl acetate=10/1 to 0/1). 3 (300 mg, 551.89 μmol, 85% yield) was obtained as a white solid.

LCMS: RT=0.427 min, MS cal.: 543.23, [M+H]⁺=544.4

¹H NMR (400 MHz, DMSO-d6) δ=7.88 (d, J=10.0 Hz, 1H), 7.76 (s, 1H), 7.72-7.67 (m, 2H), 7.63 (t, J=7.6 Hz, 1H), 7.26 (s, 1H), 6.87 (d, J=7.2 Hz, 1H), 6.70 (d, J=8.4 Hz, 1H), 5.44 (s, 2H), 4.24 (q, J=7.2 Hz, 2H), 3.89 (s, 3H), 3.79 (s, 2H), 2.90 (d, J=11.2 Hz, 2H), 2.61-2.52 (m, 1H), 2.16 (t, J=10.4 Hz, 2H), 1.80-1.56 (m, 4H), 1.41 (t, J=6.8 Hz, 3H)

### General Procedure for Preparation of Compound 261 (4):

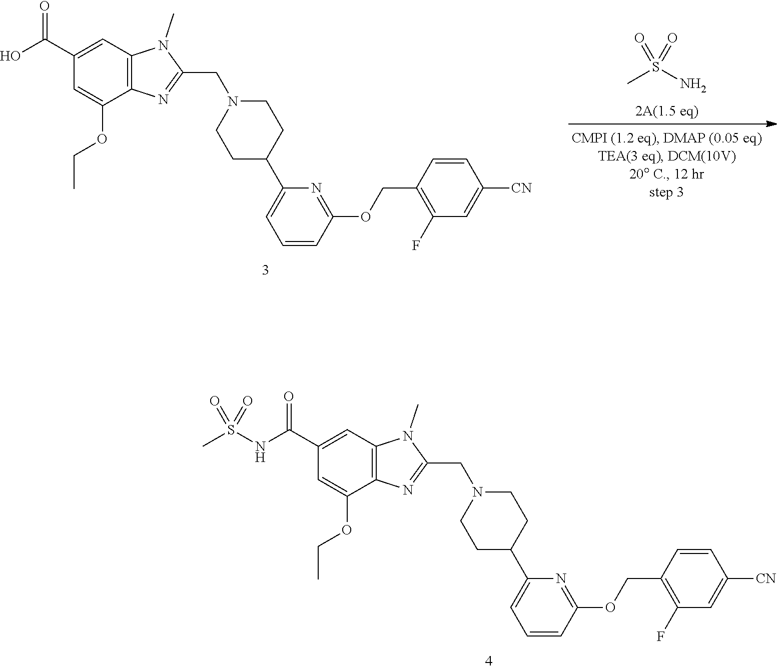

To a solution of 3 (100 mg, 183.96 μmol, 1 eq) in DCM (1 mL) was added DMAP (1.12 mg, 9.20 μmol, 0.05 eq) and TEA (55.85 mg, 551.89 μmol, 76.82 μL, 3 eq) and 2-chloro-CMPI (56.40 mg, 220.76 μmol, 1.2 eq) at 20° C. for 0.5 hr. The mixture was added 2A (26.25 mg, 275.94 μmol, 1.5 eq) and stirred at 20° C. for 11.5 hr. LC-MS (Rt=1.107) showed 3 was consumed completely and desired mass was detected. The reaction mixture was added H₂O (10 mL) at 0° C., and then extracted with DCM (5 mL*3). The combined organic layers were washed with brine (5 mL*2), dried over Na₂SO₄, filtered, and concentrated under reduced pressure to give a crude product. The crude product was purified by prep-HPLC (column: Waters Xbridge Prep OBD C18 150*40 mm*10 um; mobile phase: [H₂O (10 mM NH₄HCO₃)-ACN]; gradient: 20%-60% B over 8.0 min). 2-((4-(6-((4-Cyano-2-fluorobenzyl)oxy)pyridin-2-yl)piperidin-1-yl)methyl)-4-ethoxy-1-methyl-N-(methylsulfonyl)-1H-benzo[d]imidazole-6-carboxamide (28.12 mg, 45.30 μmol, 24.63% yield) was obtained as a white solid.

LCMS: RT=1.107 min, MS cal.: 620.22, [M+H]⁺=621.1

HPLC: RT=10.467, purity: 100.00%

¹H NMR (400 MHz, DMSO-d6) δ=12.08-11.46 (m, 1H), 7.92-7.86 (m, 2H), 7.73-7.68 (m, 2H), 7.65 (t, J=7.6 Hz, 1H), 7.31 (s, 1H), 6.89 (d, J=7.6 Hz, 1H), 6.72 (d, J=8.0 Hz, 1H), 5.46 (s, 2H), 4.26 (q, J=6.8 Hz, 2H), 4.05-3.82 (m, 5H), 3.26 (s, 3H), 3.01 (s, 2H), 2.70-2.56 (m, 1H), 2.42-2.21 (m, 2H), 1.88-1.63 (m, 4H), 1.42 (t, J=6.8 Hz, 3H)

### Example A251—Preparation of Compound 260. 4-(((6-(1-((4-ethoxy-1-methyl-6-(5-oxo-2,5-dihydro-1,2,4-oxadiazol-3-yl)-1H-benzo[d]imidazol-2-yl)methyl)piperidin-4-yl)pyridin-2-yl)oxy)methyl)-3-fluorobenzonitrile (Compound 260) (9)

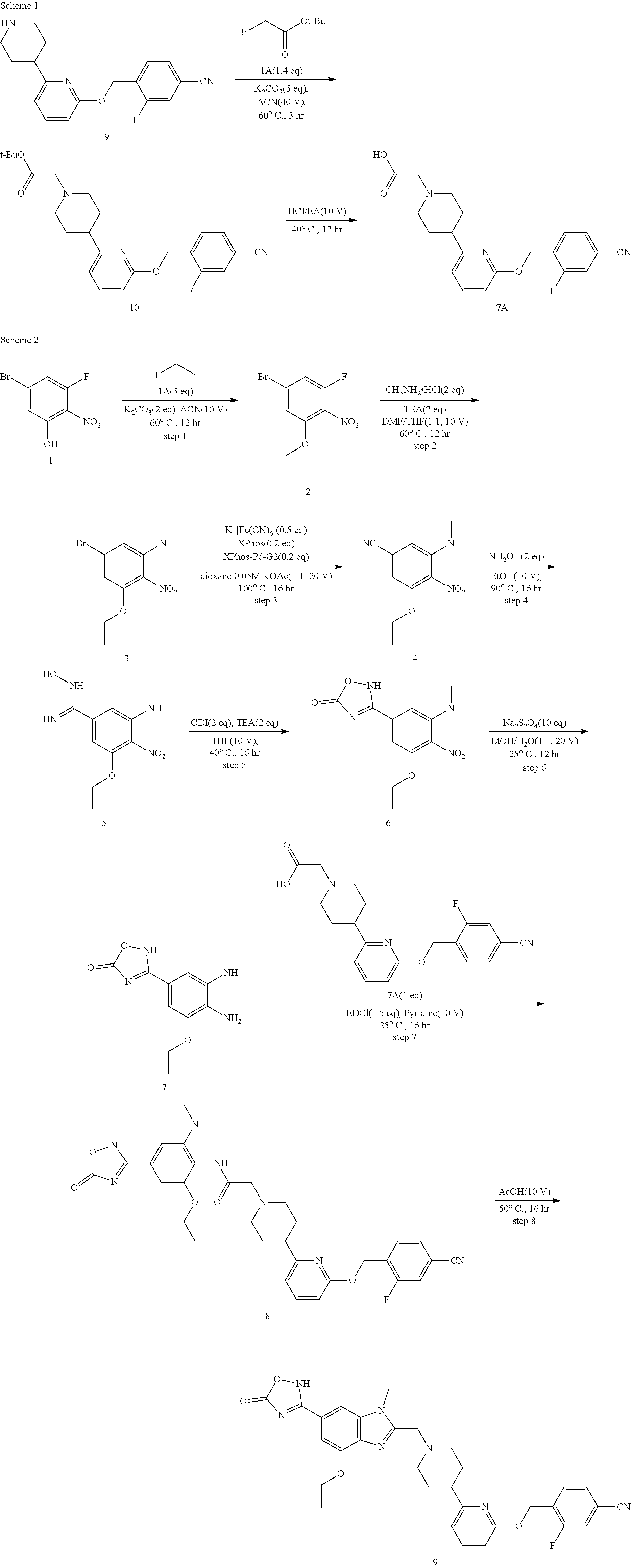

### General Procedure for Preparation of Intermediate 10:

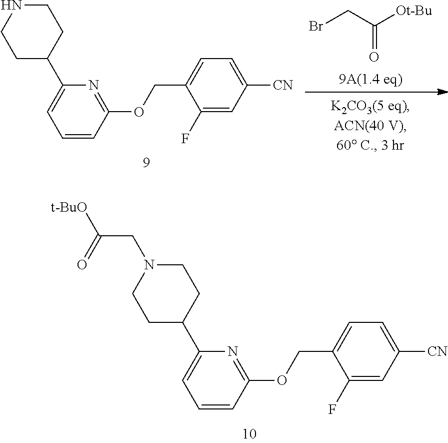 [SMILES: N#Cc1ccc(COc2cccc(C3CCNCC3)n2)c(F)c1 ; CC(C)(C)OC(=O)CBr ; CC(C)(C)OC(=O)CN1CCC(c2cccc(OCc3ccc(C#N)cc3F)n2)CC1]

To a solution of 9 (3 g, 9.64 mmol, 1 eq) and 9A (2.63 g, 13.49 mmol, 1.99 mL, 1.4 eq) in MeCN (120 mL) was added K₂CO₃ (6.66 g, 48.18 mmol, 5 eq) at 15° C., the mixture was stirred at 60° C. for 3 hr. LCMS showed 9 was consumed completely and one main peak with desired was detected. The reaction mixture was diluted with H₂O (100 mL) and extracted with EA (100 mL*3). The combined organic layers were washed with brine (100 mL*2), dried over Na₂SO₄, filtered, and concentrated under reduced pressure to give a residue. The residue was purified by column chromatography (SiO₂, PE/EA=1/0 to 7/3). 10 (3.2 g, 7.52 mmol, 78.05% yield) was obtained as a white solid.

¹H NMR (400 MHz, CHLOROFORM-d) δ=7.64 (t, J=7.2 Hz, 1H), 7.53 (t, J=7.6 Hz, 1H), 7.45 (d, J=7.6 Hz, 1H), 7.38 (d, J=9.2 Hz, 1H), 6.77 (d, J=7.2 Hz, 1H), 6.64 (d, J=8.4 Hz, 1H), 5.51 (s, 2H), 3.17 (s, 2H), 3.07 (d, J=11.2 Hz, 2H), 2.57 (tt, J=3.6, 11.6 Hz, 1H), 2.31 (t, J=11.2 Hz, 2H), 1.95 (dq, J=3.6, 12.4 Hz, 2H), 1.88-1.80 (m, 2H), 1.49 (s, 9H)

### General Procedure for Preparation of Intermediate 7A:

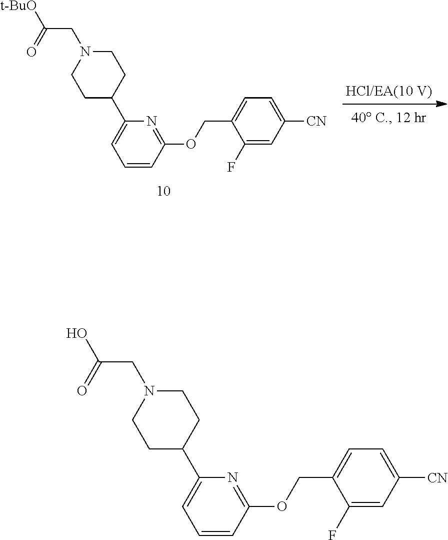 [SMILES: CC(C)(C)OC(=O)CN1CCC(c2cccc(OCc3ccc(C#N)cc3F)n2)CC1 ; N#Cc1ccc(COc2cccc(C3CCN(CC(=O)O)CC3)n2)c(F)c1]

A mixture of 10 (1.6 g, 3.76 mmol, 1 eq) in HCl/EA (4 M, 16.00 mL, 17.02 eq) at 15° C., the mixture was stirred at 40° C. for 12 hr. LC-MS showed 10 was consumed completely and one main peak with desired was detected. The mixture was concentrated under reduced pressure to give residue. 7A (3.2 g, crude) was obtained as a white solid.

¹H NMR (400 MHz, DMSO-d6) δ=10.20 (d, J=3.2 Hz, 1H), 7.90 (d, J=9.6 Hz, 1H), 7.81-7.75 (m, 1H), 7.75-7.68 (m, 2H), 6.92 (d, J=7.2 Hz, 1H), 6.78 (d, J=8.0 Hz, 1H), 5.49 (s, 2H), 4.14 (d, J=4.0 Hz, 2H), 3.62 (d, J=10.4 Hz, 2H), 3.26-3.12 (m, 2H), 2.86 (t, J=10.8 Hz, 1H), 2.16-2.00 (m, 4H).

### General Procedure for Preparation of Intermediate 2:

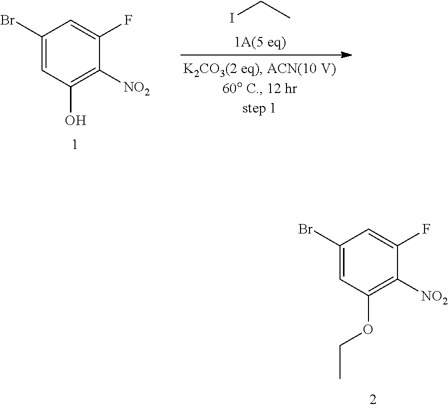

To a mixture of 1 (10 g, 42.37 mmol, 1 eq) and 1A (33.04 g, 211.87 mmol, 16.95 mL, 5 eq) in MeCN (100 mL) was added K₂CO₃ (11.71 g, 84.75 mmol, 2 eq). The mixture was stirred at 60° C. for 12 hr under N₂ atmosphere. LC-MS showed 1 was consumed completely and desired mass was detected. The suspension was filtered, and the filtrate was concentrated under reduced pressure to give a residue. 2 (10 g, crude) was obtained as a yellow solid, which was used directly for the next step.

¹H NMR (400 MHz, DMSO-d6) δ=7.55 (dd, J=1.6, 9.6 Hz, 1H), 7.50 (s, 1H), 4.27 (q, J=6.8 Hz, 2H), 1.30 (t, J=6.8 Hz, 3H)

### General Procedure for Preparation of Intermediate 3:

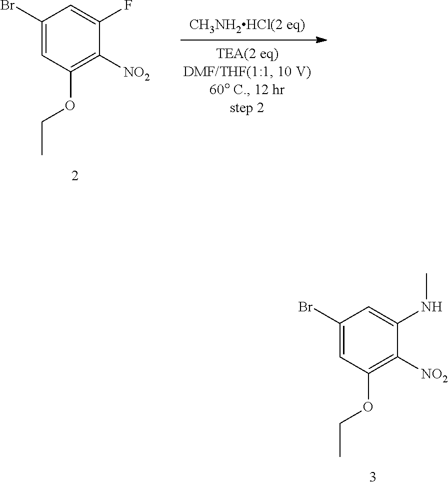

To a solution of 2 (10 g, 37.87 mmol, 1 eq) in DMF (50 mL) and THF (50 mL) was added CH₃NH₂·HCl (5.11 g, 75.74 mmol, 2 eq) and TEA (7.66 g, 75.74 mmol, 10.54 mL, 2 eq). The mixture was stirred at 60° C. for 12 hr. LC-MS showed 2 was consumed completely and desired mass was detected. The residue was poured into H₂O (200 mL) and extracted with ethyl acetate (100 mL*3). The combined organic layers were washed with brine (100 mL*3), dried over Na₂SO₄, filtered, and concentrated under reduced pressure to give a residue. The residue was purified by column chromatography (SiO₂, Petroleum ether/Ethyl acetate=20/1 to 10/1). 3 (8.1 g, 29.44 mmol, 77.75% yield) was obtained as a yellow solid.

¹H NMR (400 MHz, DMSO-d6) δ=6.61 (d, J=1.6 Hz, 1H), 6.55 (d, J=1.6 Hz, 1H), 6.42 (q, J=4.8 Hz, 1H), 4.10 (q, J=6.8 Hz, 2H), 2.72 (d, J=4.8 Hz, 3H), 1.25 (t, J=6.8 Hz, 3H).

### General Procedure for Preparation of Intermediate 4:

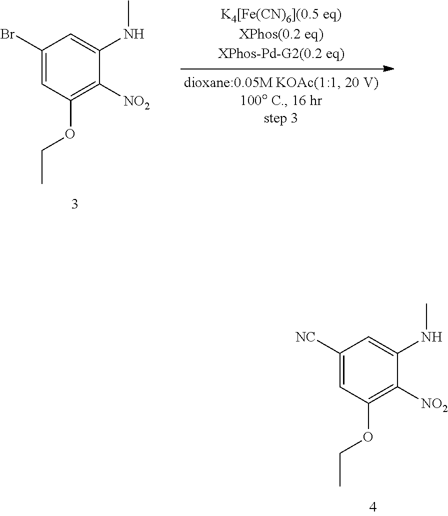 [SMILES: CCOc1cc(Br)cc(NC)c1[N+](=O)[O-] ; CCOc1cc(C#N)cc(NC)c1[N+](=O)[O-]]

To a mixture of 3 (2 g, 7.27 mmol, 1 eq), K₄[Fe(CN)₆] (1.34 g, 3.64 mmol, 0.5 eq) and XPhos (693.16 mg, 1.45 mmol, 0.2 eq) in dioxane (20 mL) and KOAc (0.05 M, 20 mL) was added XPhos-Pd-G2 (1.14 g, 1.45 mmol, 0.2 eq) under N₂ atmosphere. The mixture was degassed and purged with N₂ for 3 times, and stirred under N₂ atmosphere at 100° C. for 16 hr. And the reaction was set 4 batches on parallel. LC-MS showed 3 was consumed completely and desired mass was detected. The reaction mixture was cooled to 25° C. and combined together, poured into H₂O (50 mL) and extracted with EtOAc (50 mL*3). And the combined organic layers were dried over Na₂SO₄, filtered, and concentrated under reduced pressure to give a residue. The residue was purified by column chromatography (SiO₂, Petroleum ether/Ethyl acetate=10/1 to 3/1). 4 (7.1 g, 19.26 mmol, 66.22% yield, 60% purity) was obtained as a yellow solid.

¹H NMR (400 MHz, DMSO-d6) δ=6.91 (d, J=0.8 Hz, 1H), 6.85 (d, J=0.8 Hz, 1H), 6.46 (q, J=4.8 Hz, 1H), 4.15 (q, J=6.8 Hz, 2H), 2.73 (d, J=4.8 Hz, 3H), 1.26 (t, J=6.8 Hz, 3H)

### General Procedure for Preparation of Intermediate 5:

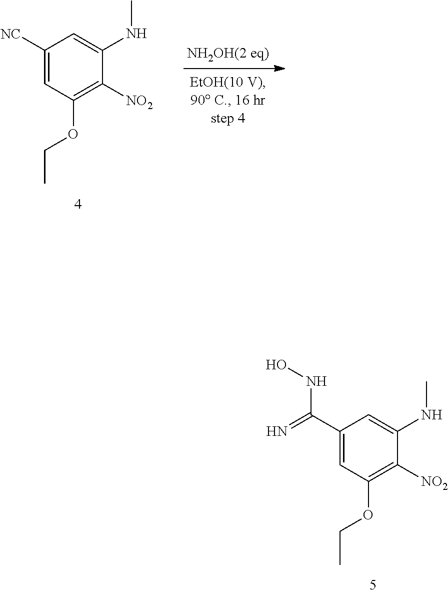 [SMILES: CCOc1cc(C#N)cc(NC)c1[N+](=O)[O-] ; CCOc1cc(C(=N)NO)cc(NC)c1[N+](=O)[O-]]

To a solution of 4 (5 g, 22.60 mmol, 60% purity, 1 eq) in EtOH (50 mL) was added hydroxylamine (2.99 g, 45.21 mmol, 50% purity, 2 eq) at 15° C., and the mixture was stirred at 90° C. for 16 hr. LC-MS showed 4 was consumed completely and desired mass was detected. The reaction mixture was concentrated under reduced pressure to give a residue. The residue was triturated with MTBE (10 V) at 15° C. for 20 min, filtered and washed by MTBE (50 mL*3), and the filter cake was further purified by prep-HPLC (basic condition; column: Waters Xbridge BEH C18 250*70 mm*10 um; mobile phase: [H₂O (10 mM NH₄HCO₃)-ACN]; gradient: 20%-50% B over 20.0 min). 5 (3.8 g, 14.95 mmol, 66.13% yield) was obtained as a yellow solid.

¹H NMR (400 MHz, DMSO-d6) δ=9.82 (s, 1H), 6.68 (s, 2H), 6.25 (q, J=4.8 Hz, 1H), 5.92 (s, 2H), 4.10 (q, J=6.8 Hz, 2H), 2.77 (d, J=4.8 Hz, 3H), 1.28 (t, J=6.8 Hz, 3H)

### General Procedure for Preparation of Intermediate 6:

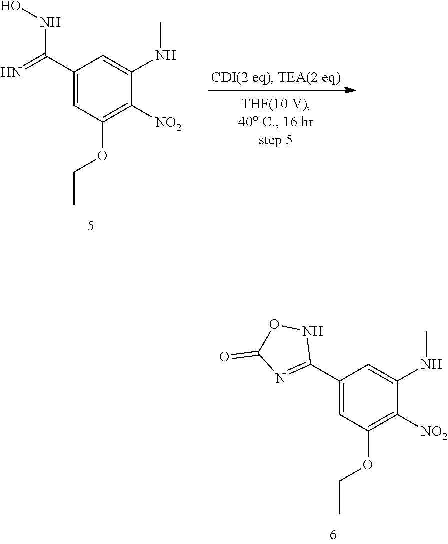 [SMILES: CCOc1cc(C(=N)NO)cc(NC)c1[N+](=O)[O-] ; CCOc1cc(-c2nc(=O)o[nH]2)cc(NC)c1[N+](=O)[O-]]

To a solution of 5 (1.5 g, 5.90 mmol, 1 eq) in THF (15 mL) was added CDI (1.91 g, 11.80 mmol, 2 eq) and TEA (1.19 g, 11.80 mmol, 1.64 mL, 2 eq) at 15° C., and the mixture was stirred at 40° C. for 16 hr. LC-MS showed 5 was consumed completely and desired mass was detected. The reaction mixture was concentrated under reduced pressure to give a residue, and then diluted with H₂O (20 mL) and extracted with DCM/i-PrOH (2:1, 20 mL*5). The combined organic layers were washed with brine (20 mL*2), dried over Na₂SO₄, filtered and concentrated under reduced pressure to give a residue. The residue was triturated with EtOAc (10 V) at 15° C. for 20 min. After filtration, the filter cake was dried under reduced pressure. 6 (1.11 g, 3.96 mmol, 67.14% yield) was obtained as an orange solid.

¹H NMR (400 MHz, DMSO-d6) δ=7.93 (s, 1H), 6.80 (s, 1H), 6.78 (s, 1H), 6.40 (q, J=3.6 Hz, 1H), 4.13 (q, J=6.8 Hz, 2H), 2.77 (d, J=3.6 Hz, 3H), 1.29 (t, J=6.8 Hz, 3H)

### General Procedure for Preparation of Intermediate 7:

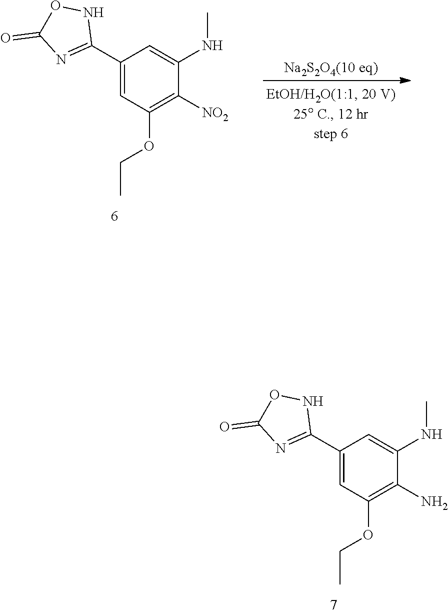 [SMILES: CCOc1cc(-c2nc(=O)o[nH]2)cc(NC)c1[N+](=O)[O-] ; CCOc1cc(-c2nc(=O)o[nH]2)cc(NC)c1N]

To a solution of 6 (430 mg, 920.65 μmol, 1 eq) in EtOH (4.3 mL) was added a solution of Na₂S₂O₄ (1.60 g, 9.21 mmol, 10 eq) in H₂O (4.3 mL) at 0° C., and the mixture was warmed to 25° C. stirred at this temperature for 12 hr. LC-MS showed 6 was consumed completely and desired mass was detected. The reaction mixture was concentrated under reduced pressure to give a residue, and then diluted with H₂O (5 mL) and extracted with DCM/i-PrOH (2:1, 10 mL*5). The combined organic layers were washed with brine (30 mL*2), dried over Na₂SO₄, filtered and concentrated under reduced pressure to give a residue. 7 (183 mg, crude) was obtained as an orange solid.

LCMS: RT=0.835 min, MS cal.: 250.1, [M+H]⁺=251.3

### General Procedure for Preparation of Intermediate 8:

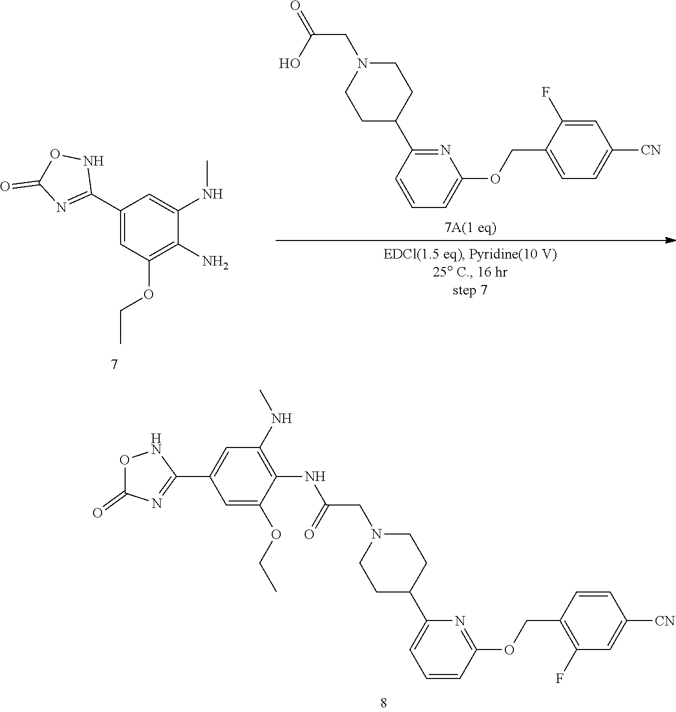 [SMILES: CCOc1cc(-c2nc(=O)o[nH]2)cc(NC)c1N ; N#Cc1ccc(COc2cccc(C3CCN(CC(=O)O)CC3)n2)c(F)c1 ; CCOc1cc(-c2nc(=O)o[nH]2)cc(NC)c1NC(=O)CN1CCC(c2cccc(OCc3ccc(C#N)cc3F)n2)CC1]

To a mixture of 7 (150 mg, 359.64 μmol, 1 eq), 7A (132.85 mg, 359.64 μmol, 1 eq) in Pyridine (1.5 mL) was added EDCI (103.41 mg, 539.45 μmol, 1.5 eq). The mixture was stirred at 25° C. for 16 hr. LC-MS showed 7 was consumed completely and desired mass was detected. The reaction mixture was diluted with H₂O (10 mL) and extracted with DCM/i-PrOH (3:1, 10 mL*5). The combined organic layers were washed with FA (0.5 M in H₂O, 30 mL*3), dried over Na₂SO₄, filtered, and concentrated under reduced pressure to give a residue. 8 (183 mg, crude) was obtained as a brown solid.

LCMS: RT=1.368 min, MS cal.: 601.2, [M+H]⁺=602.5

### General Procedure for Preparation of Compound 260 (9):

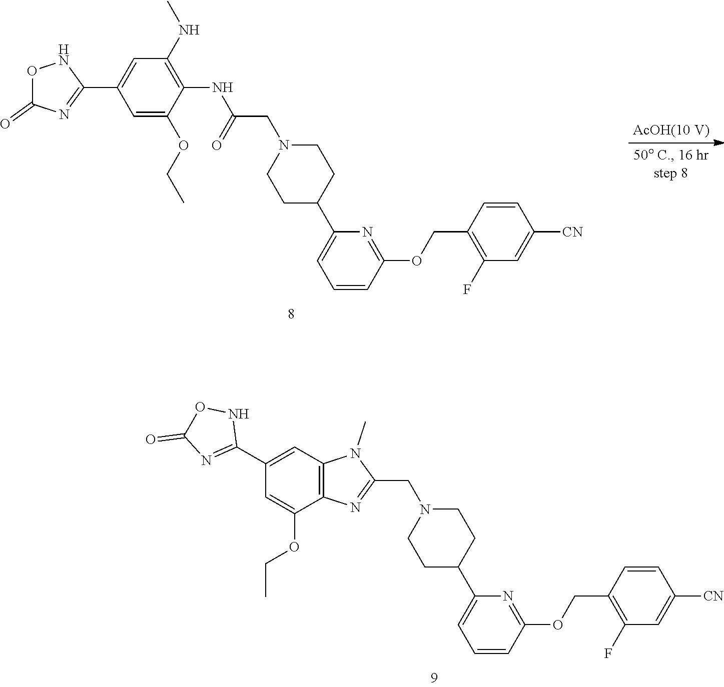 [SMILES: CCOc1cc(-c2nc(=O)o[nH]2)cc(NC)c1NC(=O)CN1CCC(c2cccc(OCc3ccc(C#N)cc3F)n2)CC1 ; CCOc1cc(-c2nc(=O)o[nH]2)cc2c1nc(CN1CCC(c3cccc(OCc4ccc(C#N)cc4F)n3)CC1)n2C]

A mixture of 8 (50 mg, 83.11 μmol, 1 eq) in AcOH (0.5 mL) was stirred at 50° C. for 16 hr. LC-MS showed 8 was consumed completely and desired mass was detected. The reaction mixture was concentrated under reduced pressure to remove solvent. The residue was diluted with sat. aq. NaHCO₃ (2 mL) and extracted with EtOAc (5 mL*3). The combined organic layers were washed with brine (5 mL*2), dried over Na₂SO₄, filtered and concentrated under reduced pressure to give a residue. The residue was purified by prep-HPLC (acidic condition; column: Phenomenex luna C18 100*40 mm*5 um; mobile phase: [H₂O (0.2% FA)-ACN]; gradient: 1%-50% B over 8.0 min). 4-(((6-(1-((4-ethoxy-1-methyl-6-(5-oxo-2,5-dihydro-1,2,4-oxadiazol-3-yl)-1H-benzo[d]imidazol-2-yl)methyl)piperidin-4-yl)pyridin-2-yl)oxy)methyl)-3-fluorobenzonitrile (29.8 mg, 51.10 μmol, 61.48% yield, 99.42% purity) was obtained as a white solid.

LCMS: RT=2.196 min, MS cal.: 583.2, [M+H]⁺=584.2

HPLC: RT=10.694 min, purity: 99.42%

¹H NMR (400 MHz, METHANOL-d4) δ=7.66 (t, J=7.2 Hz, 1H), 7.62-7.52 (m, 4H), 7.18 (d, J=1.2 Hz, 1H), 6.84 (d, J=7.2 Hz, 1H), 6.69 (d, J=8.4 Hz, 1H), 5.51 (s, 2H), 4.31 (q, J=7.2 Hz, 2H), 4.02 (s, 2H), 3.98 (s, 3H), 3.13 (d, J=11.6 Hz, 2H), 2.78-2.66 (m, 1H), 2.50-2.44 (m, 2H), 1.89-1.84 (m, 4H), 1.54 (t, J=7.2 Hz, 3H)

### Example A252—Preparation of Compound 259. 2-(2-((4-(6-((4-Cyano-2-fluorobenzyl)oxy)pyridin-2-yl)piperidin-1-yl)methyl)-4-ethoxy-1-methyl-1H-benzo[d]imidazol-6-yl) acetic acid (Compound 259) (6)

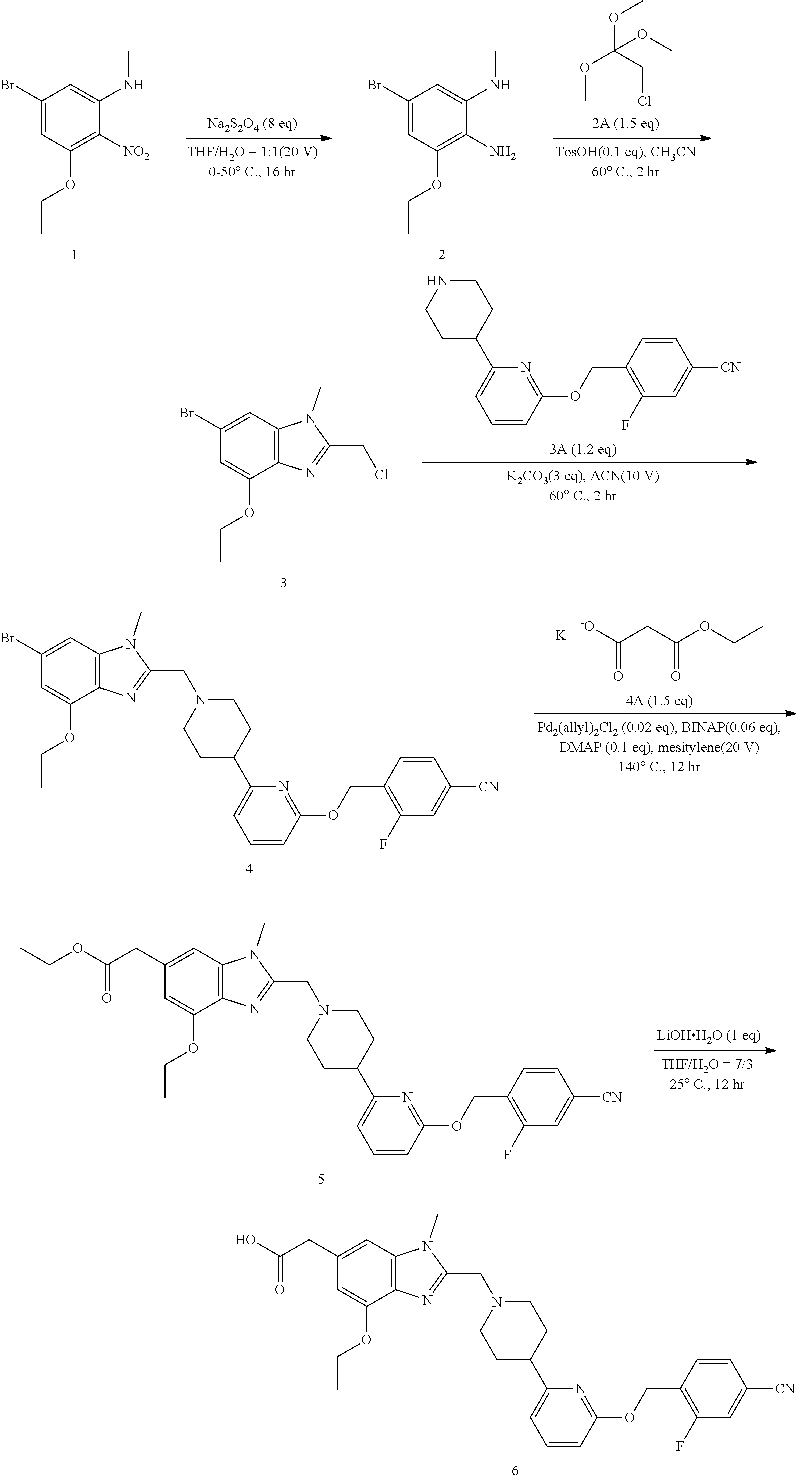

### General Procedure for Preparation of Intermediate 2:

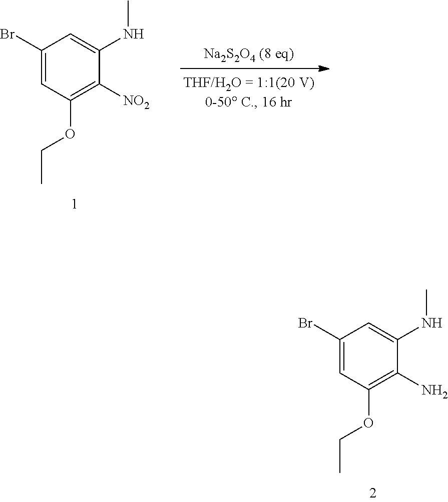

Na₂S₂O₄ (5.06 g, 29.08 mmol, 6.33 mL, 8 eq) in H₂O (20 mL) was added to the solution of 1 (1 g, 3.64 mmol, 1 eq) in THF (20 mL) at 0° C. Then the solution was stirred at 50° C. for 16 h. TLC (Petroleum ether:Ethyl acetate=3:1, Rf=0.43) indicated 1 was consumed completely and one new spot formed. The reaction was clean according to TLC. The reaction mixture was diluted with H₂O 20 mL and extracted with EtOAc 60 mL (20 mL*3). The combined organic layers were washed with Brine 15 mL, dried over Na₂SO₄, filtered, and concentrated under reduced pressure to give a residue. 2 (810 mg, 3.30 mmol, 90.91% yield) was obtained as a yellow solid.

1H NMR (400 MHz, CHLOROFORM-d) δ=6.60-6.42 (m, 2H), 4.04 (q, J=7.2 Hz, 2H), 3.45-3.17 (m, 3H), 2.85 (s, 3H), 1.42 (t, J=7.2 Hz, 3H)

### General Procedure for Preparation of Intermediate 3:

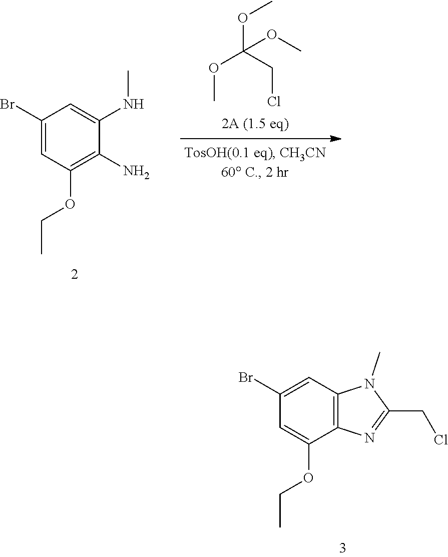

A mixture of 2 (810 mg, 3.30 mmol, 1 eq), 2A (766.29 mg, 4.96 mmol, 668.08 μL, 1.5 eq), TosOH (56.90 mg, 330.46 μmol, 0.1 eq) in ACN (8 mL) was degassed and purged with N₂ for 3 times, and then the mixture was stirred at 60° C. for 2 hr under N₂ atmosphere. LC-MS (RT=1.218 min) showed 2 was consumed completely and one main peak with desired mass was detected. The reaction mixture was concentrated under reduced pressure to give a residue. 3 (1 g, 3.29 mmol, 99.68% yield) was obtained as a yellow solid.

LCMS: RT=1.218 min, MS cal.: 301.98 [M+1]⁺=304.8

1H NMR (400 MHz, CHLOROFORM-d) δ=7.14 (d, J=1.6 Hz, 1H), 6.85 (d, J=1.2 Hz, 1H), 4.91 (s, 2H), 4.24 (q, J=7.2 Hz, 2H), 3.84 (s, 3H), 1.55 (t, J=7.2 Hz, 3H)

### General Procedure for Preparation of Intermediate 4:

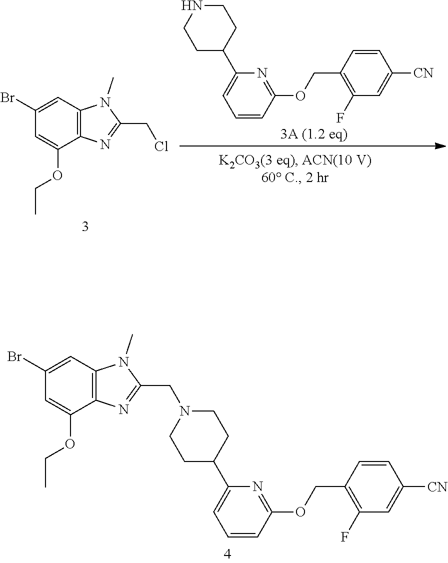 [SMILES: CCOc1cc(Br)cc2c1nc(CCl)n2C ; N#Cc1ccc(COc2cccc(C3CCNCC3)n2)c(F)c1 ; CCOc1cc(Br)cc2c1nc(CN1CCC(c3cccc(OCc4ccc(C#N)cc4F)n3)CC1)n2C]

A mixture of 3 (500 mg, 1.65 mmol, 1 eq), 3A (615.36 mg, 1.98 mmol, 1.2 eq), K₂CO₃ (682.87 mg, 4.94 mmol, 3 eq) in CH₃CN (5 mL) was degassed and purged with N₂ for 3 times, and then the mixture was stirred at 60° C. for 2 hr under N₂ atmosphere. LC-MS (product, RT=1.685 min) showed 3 was consumed completely and one main peak with desired mass was detected. The reaction mixture was diluted with H₂O 15 mL and extracted with EtOAc 60 mL (20 mL*3). The combined organic layers were washed with Brine 10 mL, dried over Na₂SO₄, filtered, and concentrated under reduced pressure to give a residue. The residue was purified by column chromatography (SiO₂, Petroleum ether/Ethyl acetate=10/1 to 0/1). 4 (900 mg, 1.56 mmol, 94.46% yield) was obtained as a yellow solid.

LCMS: RT=1.218 min, MS cal.: 577.15 [M+1]⁺=578.1

1H NMR (400 MHz, CHLOROFORM-d) δ=7.69-7.59 (m, 1H), 7.53 (t, J=7.2 Hz, 1H), 7.44 (d, J=8.0 Hz, 1H), 7.37 (dd, J=1.2, 9.2 Hz, 1H), 7.13 (d, J=1.2 Hz, 1H), 6.80 (d, J=1.2 Hz, 1H), 6.75 (d, J=7.2 Hz, 1H), 6.64 (d, J=8.0 Hz, 1H), 5.50 (s, 2H), 4.26 (q, J=7.2 Hz, 2H), 3.92-3.80 (m, 5H), 2.93 (br d, J=10.0 Hz, 2H), 2.58 (br t, J=11.2 Hz, 1H), 2.25 (br t, J=11.2 Hz, 2H), 1.94-1.65 (m, 4H), 1.55 (t, J=7.2 Hz, 3H)

### General Procedure for Preparation of Intermediate 5:

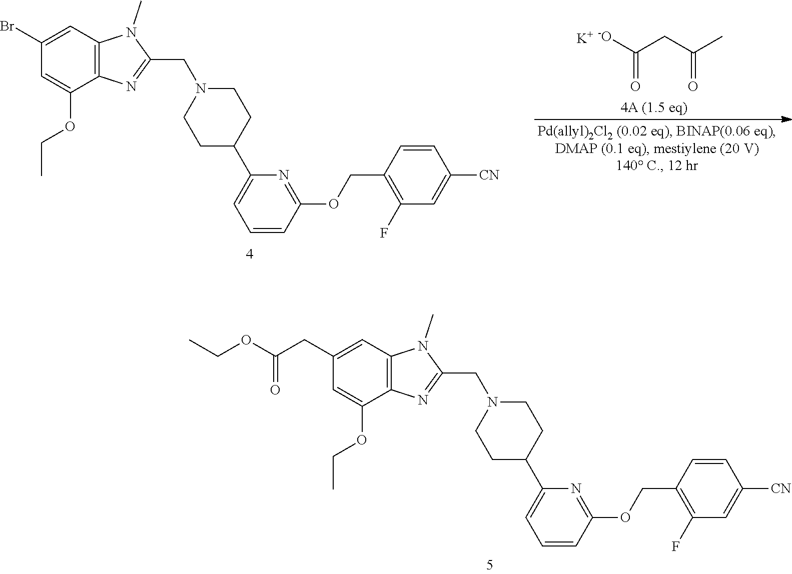

A mixture of 4 (200 mg, 345.74 μmol, 1 eq), 4A (88.27 mg, 518.61 μmol, 1.5 eq), Pd₂(allyl)₂Cl₂ (2.53 mg, 6.91 μmol, 0.02 eq), BINAP (12.92 mg, 20.74 μmol, 0.06 eq) and DMAP (4.22 mg, 34.57 μmol, 0.1 eq) in mesitylene (2 mL) was degassed and purged with N₂ for 3 times, and then the mixture was stirred at 140° C. for 12 hr under N₂ atmosphere. LC-MS (product, RT=1.637 min) showed 4 was consumed completely and one main peak with or desired mass was detected. The reaction mixture was diluted with H₂O 10 mL and extracted with EtOAc 45 mL (15 mL*3). The combined organic layers were washed with brine 10 mL, dried over Na₂SO₄, filtered and concentrated under reduced pressure to give a residue. The residue was purified by column chromatography (SiO₂, Petroleum ether/Ethyl acetate=10/1 to 0/1). 5 (50 mg, 85.37 μmol, 24.69% yield) was obtained as yellow solid.

LCMS: RT=1.218 min, MS cal.: 585.28 [M+1]=586.4

1H NMR (400 MHz, CHLOROFORM-d) δ=7.70-7.59 (m, 1H), 7.52 (t, J=7.6 Hz, 1H), 7.44 (br d, J=7.6 Hz, 1H), 7.37 (br d, J=9.2 Hz, 1H), 6.94-6.83 (m, 1H), 6.81-6.73 (m, 1H), 6.69-6.59 (m, 2H), 5.51 (s, 2H), 4.35-4.25 (m, 2H), 4.15 (qd, J=7.2, 17.6 Hz, 2H), 4.00-3.82 (m, 4H), 3.72 (s, 1H), 2.96 (br s, 2H), 2.59 (br s, 1H), 2.38-2.21 (m, 2H), 1.95-1.63 (m, 5H), 1.59-1.50 (m, 3H), 1.27 (br d, J=2.4 Hz, 3H)

### General Procedure for Preparation of Compound 259 (6):

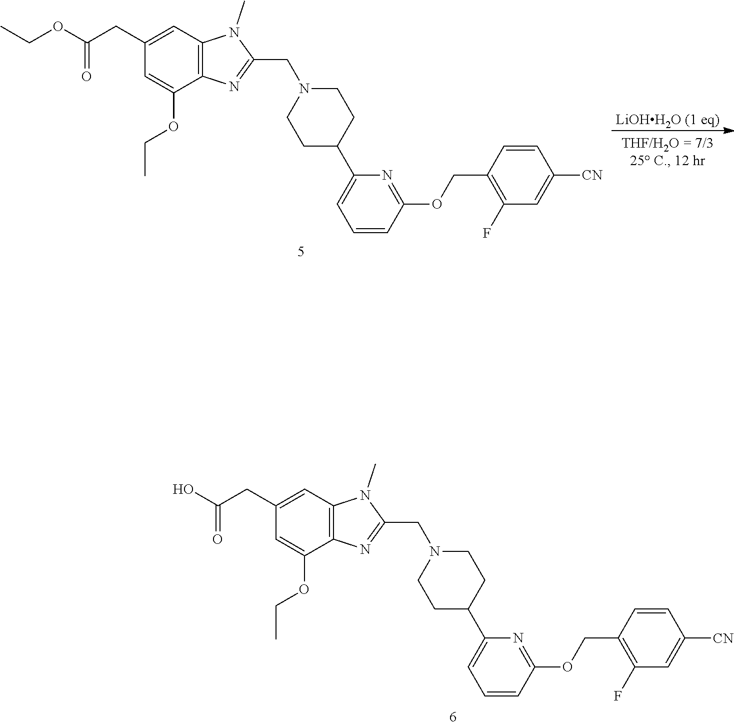 [SMILES: CCOC(=O)Cc1cc(OCC)c2nc(CN3CCC(c4cccc(OCc5ccc(C#N)cc5F)n4)CC3)n(C)c2c1 ; CCOc1cc(CC(=O)O)cc2c1nc(CN1CCC(c3cccc(OCc4ccc(C#N)cc4F)n3)CC1)n2C]

To a solution of 5 (40 mg, 68.30 μmol, 1 eq) in THF (0.7 mL) and H₂O (0.3 mL) was added LiOH·H₂O (2.87 mg, 68.30 μmol, 1 eq). The mixture was stirred at 25° C. for 12 hr. LC-MS (product, RT=1.103 min) showed 5 was consumed completely and one main peak with desired mass was detected. The reaction was purified directly by prep-HPLC (column: Waters Xbridge BEH C18 100*30 mm*10 um; mobile phase: [H₂O (10 mM NH₄HCO₃)-ACN]; gradient: 25%-55% B over 8.0 min). 2-(2-((4-(6-((4-Cyano-2-fluorobenzyl)oxy)pyridin-2-yl)piperidin-1-yl)methyl)-4-ethoxy-1-methyl-1H-benzo[d]imidazol-6-yl) acetic acid (26.8 mg, 48.00 μmol, 51.15% total yield, 99.88% purity) was obtained as a white solid.

LCMS: RT=2.283 min, MS cal.: 557.24 [M+1]=558.3

1H NMR (400 MHz, METHANOL-d4) δ=7.67 (t, J=7.6 Hz, 1H), 7.61-7.50 (m, 3H), 7.02 (s, 1H), 6.82 (d, J=7.2 Hz, 1H), 6.75 (s, 1H), 6.66 (d, J=8.0 Hz, 1H), 5.50 (s, 2H), 4.23 (q, J=7.2 Hz, 2H), 3.90 (s, 3H), 3.83 (s, 2H), 3.62 (s, 2H), 2.99 (br d, J=11.2 Hz, 2H), 2.66-2.54 (m, 1H), 2.32-2.23 (m, 2H), 1.85-1.75 (m, 4H), 1.49 (t, J=7.2 Hz, 3H)

### Example A253—Preparation of Compound 258 and Compound 271. (S)-2-((4-(2-(4-Chloro-2-fluorophenyl)-2,3-dihydrobenzo[b][1,4]dioxin-5-yl)piperidin-1-yl)methyl)-4-methoxy-1-methyl-1H-benzo[d]imidazole-6-carboxylic acid (Compound 258) (11A) & (R)-2-((4-(2-(4-Chloro-2-fluorophenyl)-2,3-dihydrobenzo[b][1,4]dioxin-5-yl)piperidin-1-yl)methyl)-4-methoxy-1-methyl-1H-benzo[d]imidazole-6-carboxylic acid (Compound 271) (11B)

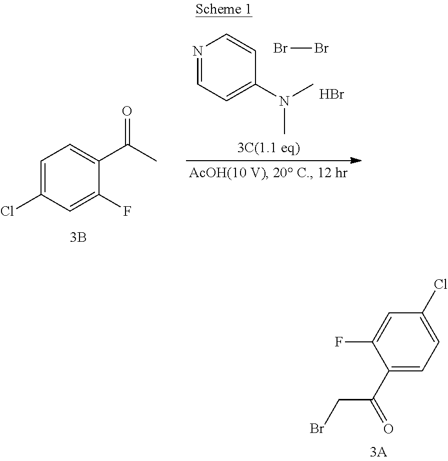

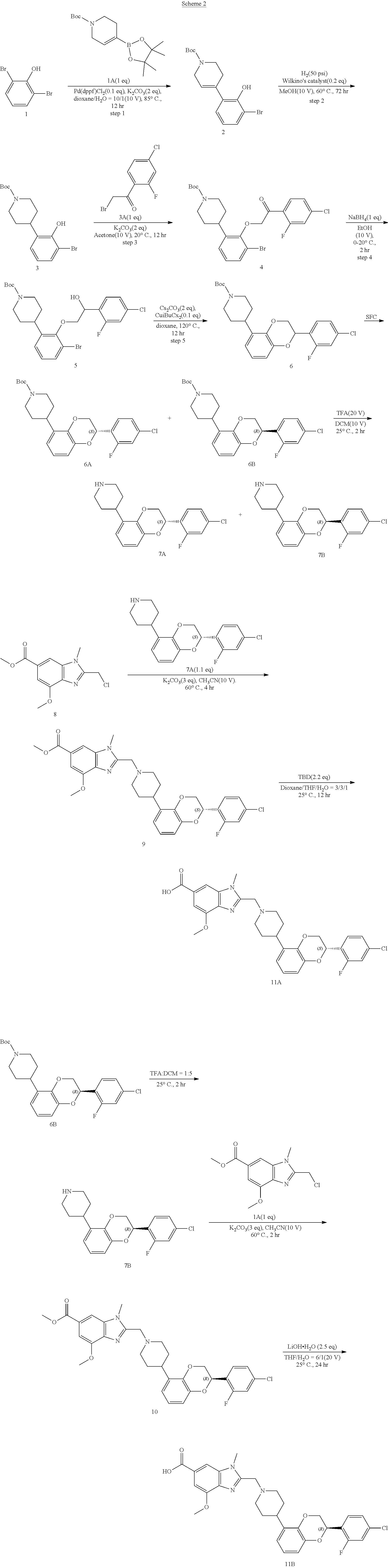

### General Procedure for Preparation of Intermediate 3A:

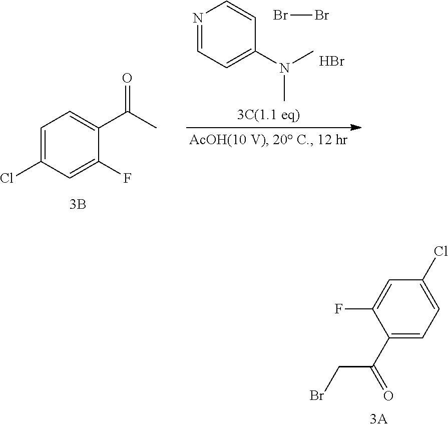

To a solution of 3B (5 g, 28.97 mmol, 1 eq) in AcOH (50 mL) was added 3C (11.56 g, 31.87 mmol, 1.1 eq). The mixture was stirred at 20° C. for 12 hr. TLC (PE:EA=10:1, Rf=0.7) showed the starting material was consumed completely. The reaction mixture was quenched by addition H₂O (100 mL) at 20° C. filtered to give a residue. 3A (6.8 g, 27.04 mmol, 93.33% yield) was obtained as a white solid.

¹H NMR (400 MHz, CHLOROFORM-d) δ=7.92 (t, J=8.0 Hz, 1H), 7.29 (dd, J=2.0, 8.8 Hz, 1H), 7.23 (dd, J=2.0, 10.8 Hz, 1H), 4.48 (d, J=2.4 Hz, 2H)

### General Procedure for Preparation of Intermediate 2:

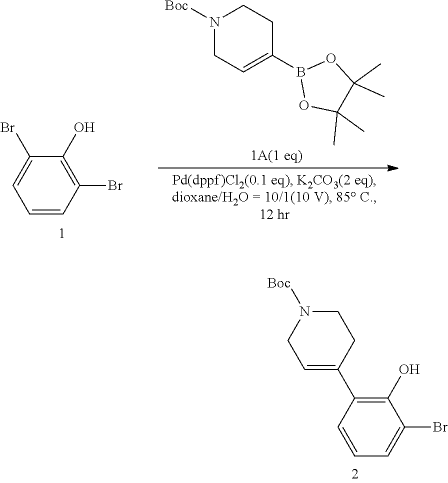 [SMILES: Oc1c(Br)cccc1Br ; CC(C)(C)OC(=O)N1CC=C(B2OC(C)(C)C(C)(C)O2)CC1 ; CC(C)(C)OC(=O)N1CC=C(c2cccc(Br)c2O)CC1]

To a solution of 1 (20 g, 79.40 mmol, 1 eq) and 1A (24.55 g, 79.40 mmol, 1 eq) in dioxane (300 mL) and H₂O (30 mL) was added K₂CO₃ (21.95 g, 158.79 mmol, 2 eq) and Pd(dppf)Cl₂ (5.81 g, 7.94 mmol, 0.1 eq). The mixture was stirred at 85° C. for 12 hr, under N₂ atmosphere. LCMS indicated 1 was consumed completely and one main peak with desired mass was detected. The reaction mixture was filtered and diluted with H₂O. Then extracted with DCM (100 mL*3). The combined organic layers were concentrated under reduced pressure to give a residue. The residue was purified by column chromatography (SiO₂, PE/EA=100/1 to 40/1). 2 (8.1 g, 22.87 mmol, 28.80% yield) was obtained as a white solid.

LCMS: RT=0.640 min, MS cal.: 298.2, [M+H]⁺=353.1

LCMS: RT=0.635 min, MS cal.: 298.2, [M+H]⁺=353.1

¹H NMR (400 MHz, CHLOROFORM-d) δ=7.39 (dd, J=1.6, 8.0 Hz, 1H), 7.09 (dd, J=1.6, 7.6 Hz, 1H), 6.79 (t, J=8.0 Hz, 1H), 5.86 (br s, 1H), 5.71 (s, 1H), 4.08 (br d, J=2.4 Hz, 2H), 3.63 (t, J=5.6 Hz, 2H), 2.51 (br s, 2H), 1.51 (s, 9H)

### General Procedure for Preparation of Intermediate 3:

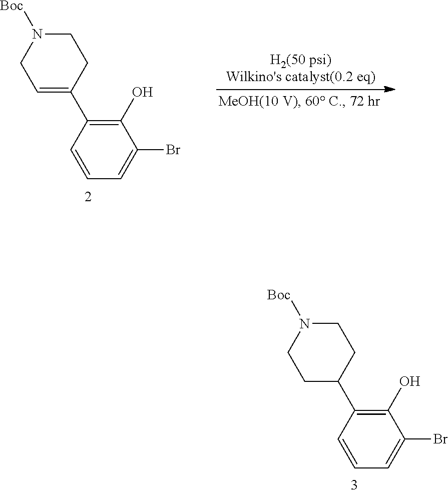 [SMILES: CC(C)(C)OC(=O)N1CC=C(c2cccc(Br)c2O)CC1 ; CC(C)(C)OC(=O)N1CCC(c2cccc(Br)c2O)CC1]

To a solution of Wilkinso's catalyst (4.23 g) in MeOH (80 mL) was added 2 (8.1 g, 22.87 mmol, 1 eq). The mixture was stirred at 60° C. for 72 hr under H₂ (50 Psi). ¹HNMR showed the starting material was consumed completely. The reaction mixture was filtered concentrated under reduced pressure. The residue was purified by column chromatography (SiO₂, PE/EA=100/1 to 60/1). 3 (3.7 g, 10.39 mmol, 45.42% yield) was obtained as a white solid.

LCMS: RT=0.635 min, MS cal.: 300.2, [M+H]⁺=355.1

¹H NMR (400 MHz, CHLOROFORM-d) δ=7.33 (dd, J=1.6, 8.0 Hz, 1H), 7.09 (d, J=6.8 Hz, 1H), 6.79 (t, J=8.0 Hz, 1H), 5.62 (s, 1H), 4.25 (d, J=11.6 Hz, 2H), 3.17-3.05 (m, 1H), 2.84 (t, J=12.4 Hz, 2H), 1.84 (d, J=13.2 Hz, 2H), 1.66-1.57 (m, 2H), 1.49 (s, 9H)

### General Procedure for Preparation of Intermediate 4:

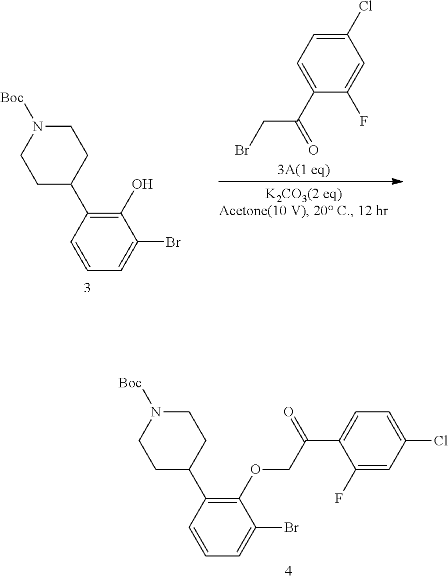 [SMILES: CC(C)(C)OC(=O)N1CCC(c2cccc(Br)c2O)CC1 ; O=C(CBr)c1ccc(Cl)cc1F ; CC(C)(C)OC(=O)N1CCC(c2cccc(Br)c2OCC(=O)c2ccc(Cl)cc2F)CC1]

To a solution of 3 (3.7 g, 10.39 mmol, 1 eq) and 3A (2.61 g, 10.39 mmol, 1 eq) in acetone (40 mL) was added K₂CO₃ (2.87 g, 20.77 mmol, 2 eq). The mixture was stirred at 20° C. for 12 hr. LCMS showed the starting material was consumed completely. The reaction mixture was diluted with H₂O and extracted with DCM 100 mL (20 mL*5). The combined organic layers were concentrated under reduced pressure to give a residue. The residue was purified by column chromatography (SiO₂, PE/EA=100/1 to 80/1). 4 (4.68 g, 8.88 mmol, 85.53% yield) was obtained as a white solid.

LCMS: RT=0.767 min, MS cal.: 426.0, [M+H]⁺=525.1

¹H NMR (400 MHz, CHLOROFORM-d) δ=8.05 (t, J=8 Hz, 1H), 7.44 (dd, J=1.6, 8.0 Hz, 1H), 7.33 (dd, J=1.6, 8.4 Hz, 1H), 7.25-7.15 (m, 2H), 7.03 (t, J=8.0 Hz, 1H), 5.31 (s, 1H), 5.16 (d, J=3.2 Hz, 2H), 4.23 (d, J=12.8 Hz, 2H), 3.24 (tt, J=3.2, 12 Hz, 1H), 2.81 (t, J=12.4 Hz, 2H), 1.77 (br d, J=12.8 Hz, 2H), 1.65-1.57 (m, 2H), 1.48 (s, 9H)

### General Procedure for Preparation of Intermediate 5:

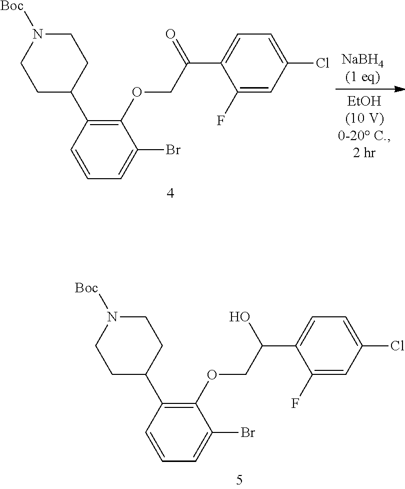 [SMILES: CC(C)(C)OC(=O)N1CCC(c2cccc(Br)c2OCC(=O)c2ccc(Cl)cc2F)CC1 ; CC(C)(C)OC(=O)N1CCC(c2cccc(Br)c2OCC(O)c2ccc(Cl)cc2F)CC1]

To a solution of 4 (4.18 g, 7.93 mmol, 1 eq) in EtOH (40 mL) was added add NaBH₄ (300.18 mg, 7.93 mmol, 1 eq) at 0° C. The resulting mixture was stirred at 20° C. for 2 hr. LCMS showed the starting material was consumed completely. The reaction mixture was added to H₂O (8 ml) at 0° C. The mixture was extracted by DCM (30 mL*3). Then the organic phase was dried with Na₂SO₄ and filtered. The organic phase was concentrated under reduced pressure at 35° C. to give a residue. The residue was purified by column chromatography (SiO₂, PE/EA=100/1 to 20/1). 5 (4 g, 7.56 mmol, 95.33% yield) was obtained as a white solid.

LCMS: RT=0.734 min, MS cal.: 428.2, [M+H]⁺=527.1

¹H NMR (400 MHz, CHLOROFORM-d) δ=7.62 (t, J=8.0 Hz, 1H), 7.42 (dd, J=1.6, 8.0 Hz, 1H), 7.22 (dd, J=2.0, 8.4 Hz, 1H), 7.16-7.08 (m, 2H), 7.03-6.96 (m, 1H), 5.52-5.44 (m, 1H), 5.31 (s, 1H), 4.33-4.18 (m, 2H), 4.12 (dd, J=2.8, 9.6 Hz, 1H), 3.10 (d, J=4.0 Hz, 1H), 2.96 (ddd, J=3.6, 8.4, 12.0 Hz, 1H), 2.81-2.63 (m, 2H), 1.68 (d, J=17.6 Hz, 2H), 1.57 (s, 2H), 1.49 (s, 9H)

### General Procedure for Preparation of Intermediate 6:

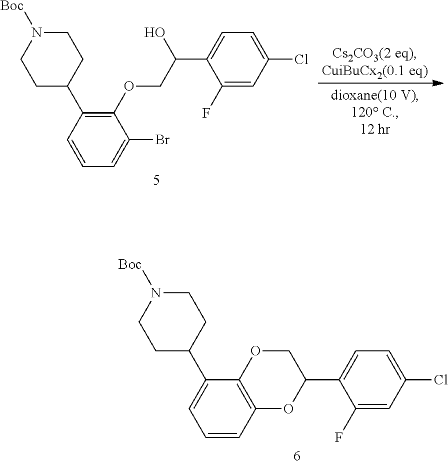 [SMILES: CC(C)(C)OC(=O)N1CCC(c2cccc(Br)c2OCC(O)c2ccc(Cl)cc2F)CC1 ; CC(C)(C)OC(=O)N1CCC(c2cccc3c2OCC(c2ccc(Cl)cc2F)O3)CC1]

To a solution of 5 (2 g, 3.78 mmol, 1 eq) in dioxane (20 mL) was added Cs₂CO₃ (2.46 g, 7.56 mmol, 2 eq) and CuiBuCx₂ (150.52 mg, 378.19 μmol, 0.1 eq). The mixture was stirred at 120° C. for 12 hr. LCMS showed the starting material was consumed completely. The reaction was filtered and concentrated under reduced pressure to give a residue. The residue was purified by column chromatography (SiO₂, PE/EA=0/1 to 1/1). 6 (3.1 g, 91.5% yield) was obtained as a white solid.

LCMS: RT=0.805 min, MS cal.: 392.2, [M+H]⁺=447.2

¹H NMR (400 MHz, CHLOROFORM-d) δ=7.47 (t, J=8.0 Hz, 1H), 7.23 (dd, J=1.6, 8.4 Hz, 1H), 7.16 (dd, J=1.6, 10.0 Hz, 1H), 6.89-6.85 (m, 2H), 6.82-6.77 (m, 1H), 5.42 (dd, J=1.9, 8.3 Hz, 1H), 4.46 (dd, J=2.4, 11.2 Hz, 1H), 4.24 (s, 2H), 3.10-2.99 (m, 1H), 2.94-2.76 (m, 2H), 1.88-1.74 (m, 2H), 1.71-1.61 (m, 2H), 1.49 (s, 9H)

### General Procedure for Preparation of Intermediate 6A & 6B:

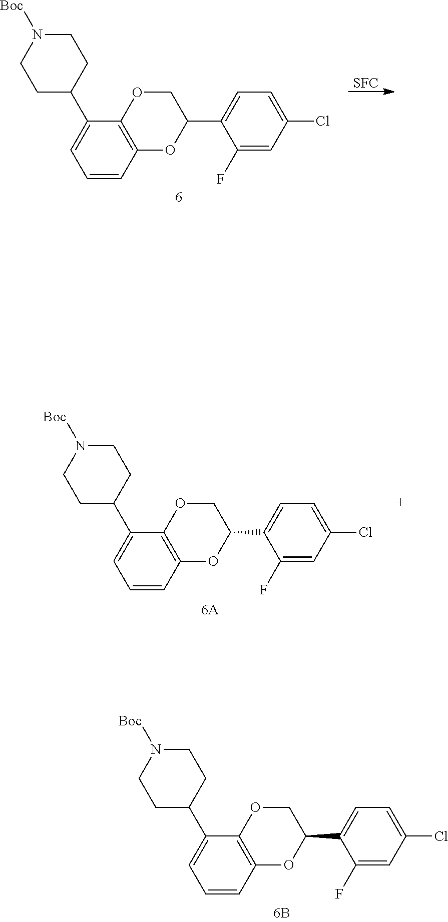 [SMILES: CC(C)(C)OC(=O)N1CCC(c2cccc3c2OCC(c2ccc(Cl)cc2F)O3)CC1 ; "SFC" ; CC(C)(C)OC(=O)N1CCC(c2cccc3c2OC[C@H](c2ccc(Cl)cc2F)O3)CC1 ; CC(C)(C)OC(=O)N1CCC(c2cccc3c2OC[C@@H](c2ccc(Cl)cc2F)O3)CC1]

6 was further separated by SFC (column: DAICEL CHIRALPAK AD (250 mm*50 mm, 10 um); mobile phase: [CO₂-EtOH (0.1% NH₃H₂O)]; B %: 11%, isocratic elution mode). 6A (410 mg, 915.33 μmol, 41.00% yield) was a white solid. 6B (220 mg, 491.15 μmol, 22.00% yield) was a white solid.

¹H NMR (400 MHz, CHLOROFORM-d) δ=7.47 (t, J=8.0 Hz, 1H), 7.23 (d, J=8.4 Hz, 1H), 7.16 (dd, J=2.0, 10.0 Hz, 1H), 6.91-6.86 (m, 2H), 6.84-6.78 (m, 1H), 5.41 (dd, J=1.6, 8.4 Hz, 1H), 4.46 (dd, J=2.4, 11.2 Hz, 1H), 4.25 (s, 2H), 3.96 (dd, J=8.4, 11.2 Hz, 1H), 3.10-2.99 (m, 1H), 2.83 (br s, 2H), 1.89-1.72 (m, 2H), 1.71-1.56 (m, 2H), 1.49 (s, 9H)

### General Procedure for Preparation of Intermediate 7A:

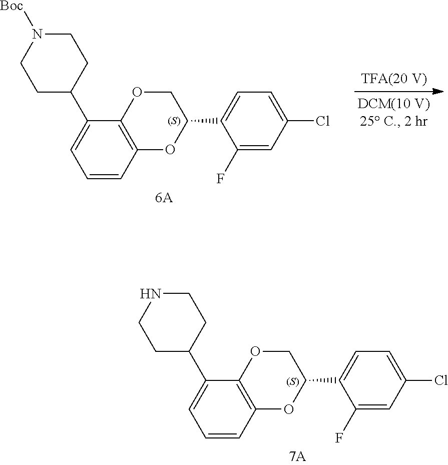

To a solution of 6A (300.00 mg, 669.75 μmol, 1 eq) in DCM (3 mL) was added TFA (921.00 mg, 8.08 mmol, 600.00 μL, 12.06 eq). The mixture was stirred at 25° C. for 2 hr. LC-MS showed 6A was consumed completely and one main peak with desired mass was detected. The mixture was concentrated in vacuum. 7A (200 mg, 575.03 μmol, 85.86% yield) was a yellow oil.

LCMS: RT=1.143 min, MS cal.: 347.11, [M+H]⁺=348.0

¹H NMR (400 MHz, CHLOROFORM-d) δ=7.47 (t, J=8.0 Hz, 1H), 7.23 (dd, J=1.6, 8.4 Hz, 1H), 7.17 (dd, J=2.0, 10.0 Hz, 1H), 6.95-6.89 (m, 2H), 6.83-6.77 (m, 1H), 5.43-5.39 (m, 1H), 5.31 (s, 1H), 4.47 (dd, J=2.4, 11.2 Hz, 1H), 3.97 (dd, J=8.4, 11.2 Hz, 1H), 3.63 (d, J=12.0 Hz, 2H), 3.23-3.08 (m, 3H), 2.17-2.00 (m, 4H)

### General Procedure for Preparation of Intermediate 9:

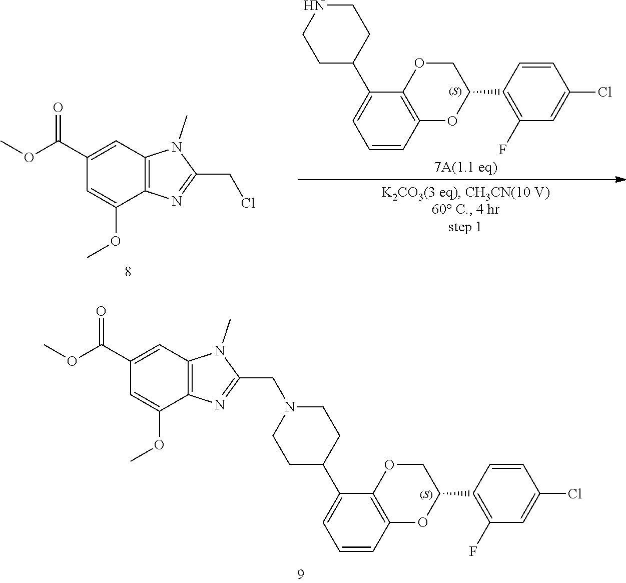

To a solution of 8 (100 mg, 372.17 μmol, 1 eq), 7A (142.39 mg, 409.39 μmol, 1.1 eq) in CH₃CN (1 mL) was added K₂CO₃ (154.31 mg, 1.12 mmol, 3 eq) at 25° C. The mixture was stirred at 60° C. for 4 hr. LC-MS showed 8 was consumed completely and one main peak with desired mass was detected. The residue was poured into water (10 mL). The aqueous phase was extracted with EA (10 mL*3). The combined organic phase was washed with brine (10 mL*2), dried with anhydrous Na₂SO₄, filtered and concentrated in vacuum. The residue was purified by column chromatography (SiO₂, PE/EA=1/0 to 0/1). 9 (130 mg, 224.12 μmol, 60.22% yield) was a white solid.

LCMS: RT=1.703 min, MS cal.: 579.19, [M+H]⁺=580.1

¹H NMR (400 MHz, CHLOROFORM-d) δ=7.78 (d, J=1.2 Hz, 1H), 7.46 (t, J=8.0 Hz, 1H), 7.39 (d, J=1.2 Hz, 1H), 7.21 (dd, J=1.6, 8.4 Hz, 1H), 7.15 (dd, J=2.0, 10.0 Hz, 1H), 6.91-6.78 (m, 3H), 5.40 (dd, J=2.0, 8.4 Hz, 1H), 4.46 (dd, J=2.4, 11.2 Hz, 1H), 4.07 (s, 3H), 3.97 (s, 6H), 3.95-3.86 (m, 3H), 3.02-2.87 (m, 3H), 2.32 (br s, 2H), 1.91-1.57 (m, 4H)

### General Procedure for Preparation of Compound 258 (11A):

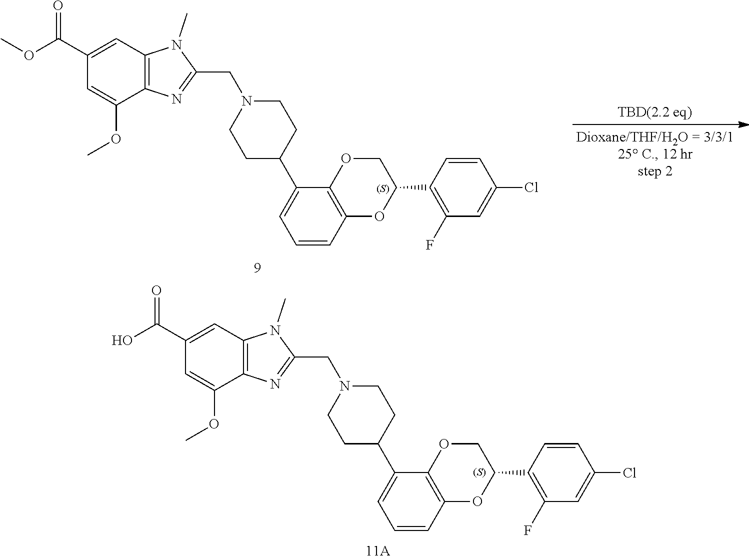

To a solution of 9 (52.80 mg, 379.28 μmol, 2.2 eq) in THF (0.5 mL), H₂O (0.1 mL), dioxane (0.5 mL) was added TBD (100 mg, 172.40 μmol, 1 eq) at 25° C. The mixture was stirred at 25° C. for 12 hr. LC-MS showed ˜11% of 9 remained. Several new peaks were shown on LC-MS and ˜81% of desired compound was detected. The mixture was filtered. The residue was purified by prep-HPLC (column: Phenomenex Luna C18 75*30 mm*3 um; mobile phase: [H₂O (0.2% FA)-ACN]; gradient: 40%-70% B over 8.0 min). (S)-2-((4-(2-(4-Chloro-2-fluorophenyl)-2,3-dihydrobenzo[b][1,4]dioxin-5-yl)piperidin-1-yl)methyl)-4-methoxy-1-methyl-1H-benzo[d]imidazole-6-carboxylic acid (12.53 mg, 22.14 μmol, 12.84% yield) was a white solid.

LCMS: RT=1.182 min, MS cal.: 565.18, [M+H]⁺=566.1

¹H NMR (400 MHz, DMSO-d6) δ=7.79 (s, 1H), 7.58-7.51 (m, 2H), 7.39 (d, J=8.8 Hz, 1H), 7.26 (s, 1H), 6.86-6.77 (m, 3H), 5.43 (d, J=6.0 Hz, 1H), 4.51 (d, J=9.2 Hz, 1H), 4.13 (dd, J=8.8, 11.6 Hz, 1H), 3.95 (s, 3H), 3.90 (s, 3H), 3.80 (s, 2H), 2.94 (d, J=11.2 Hz, 3H), 2.19 (t, J=11.2 Hz, 2H), 1.79-1.54 (m, 4H)

### General Procedure for Preparation of Intermediate 7B:

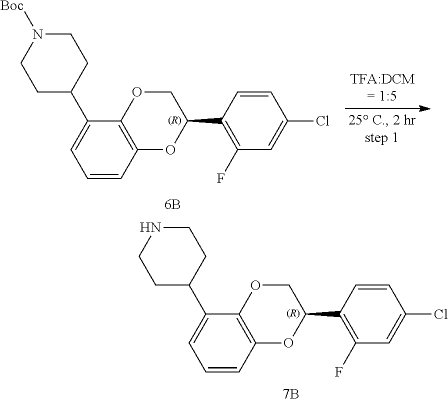

To a solution of 6B (80 mg, 178.60 μmol, 1 eq) in DCM (1 mL) was added TFA (307.00 mg, 2.69 mmol, 0.2 mL, 15.08 eq). The mixture was stirred at 25° C. for 2 hr. LCMS showed 6B was consumed completely and one main peak with desired mass was detected. The mixture was concentrated under reduced pressure. 7B (80 mg, crude, TFA) was obtained as a yellow oil. LCMS: RT=0.434 min, MS cal.: 347.1/349.1, [M+H]⁺=348.1/350.0

### General Procedure for Preparation of Intermediate 10:

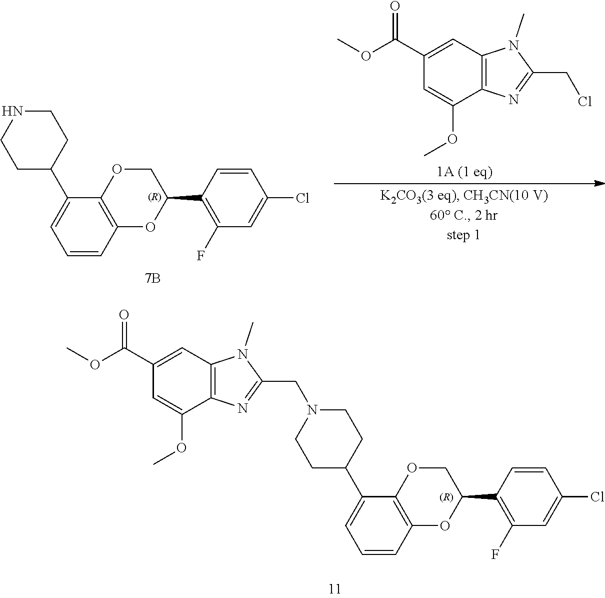

To a solution of 7B (80 mg, 173.22 μmol, 1 eq, TFA) in ACN (1 mL) was added K₂CO₃ (71.82 mg, 519.67 μmol, 3 eq) and 1A (46.54 mg, 173.22 μmol, 1 eq). The mixture was stirred at 60° C. for 2 hr. LCMS showed 7B was consumed completely and one main peak with desired mass was detected. The reaction mixture was diluted with H₂O 10 mL and extracted with EtOAc 45 mL (15 mL*3). The combined organic layers were washed with brine 20 mL, dried over Na₂SO₄, filtered, and concentrated under reduced pressure to give a residue. The residue was purified by column chromatography (SiO₂, PE/EtOAc=1/0 to 1/1). 10 (70 mg, 103.79 μmol, 59.91% yield) was obtained as a yellow solid.

LCMS: RT=1.697 min, MS cal.: 579.2/580.2, [M+H]⁺=580.2/581.1

¹HNMR (400 MHz, DMSO-d6) δ=7.84-7.81 (m, 1H), 7.58-7.50 (m, 2H), 7.41-7.36 (m, 1H), 7.26 (s, 1H), 6.84-6.79 (m, 3H), 5.45-5.40 (m, 1H), 4.54-4.48 (m, 1H), 4.13 (dd, J=8.4, 11.6 Hz, 1H), 3.96 (s, 3H), 3.91 (s, 3H), 3.88 (s, 3H), 3.81 (s, 2H), 2.98-2.80 (m, 3H), 2.19 (br t, J=10.8 Hz, 2H), 1.77-1.54 (m, 4H)

### General Procedure for Preparation of Compound 271 (11B):

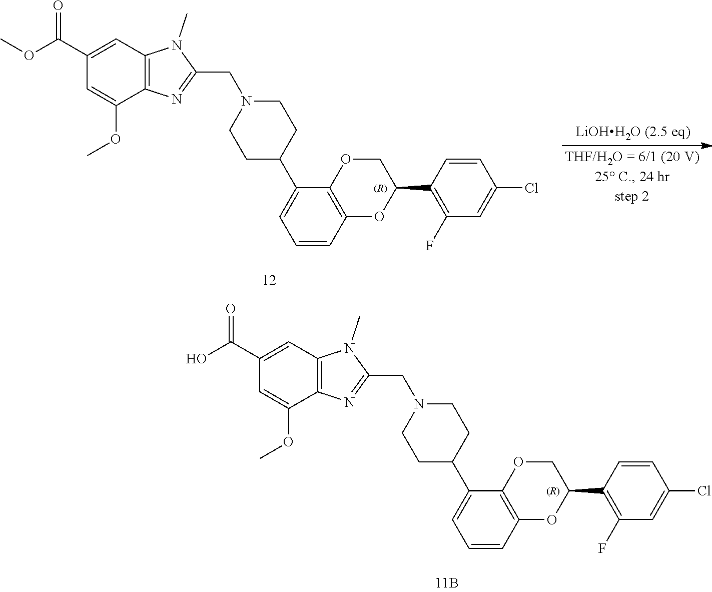

To a solution of 10 (60 mg, 103.44 μmol, 1 eq) in THF (3.6 mL) and H₂O (0.6 mL) was added LiOH·H₂O (6.51 mg, 155.16 μmol, 1.5 eq). The mixture was stirred at 25 C for 12 hr. After monitoring, the mixture was added LiOH·H₂O (4.34 mg, 103.44 μmol, 1 eq). The mixture was stirred at 25° C. for 12 hr. LCMS showed 28% of 3 remained. Several new peaks were shown on LCMS and 70% of desired compound was detected. The mixture was added 0.1 ml. The residue was purified by prep-HPLC (neutral condition: column: Waters Xbridge Prep OBD C18 150*40 mm*10 um; mobile phase: [H₂O (10 mM NH₄HCO₃)-ACN]; gradient: 35%-85% B over 8.0 min). (R)-2-((4-(2-(4-Chloro-2-fluorophenyl)-2,3-dihydrobenzo[b][1,4]dioxin-5-yl)piperidin-1-yl)methyl)-4-methoxy-1-methyl-1H-benzo[d]imidazole-6-carboxylic acid (23.75 mg, 41.54 μmol, 40.16% total yield) was obtained as a white solid.

LCMS: RT=2.768 min, MS cal.: 565.2/566.2, [M+H]⁺=566.2/567.2

HPLC: RT=12.119 min

¹HNMR (400 MHz, DMSO-d6) δ=7.75-7.70 (m, 1H), 7.59-7.50 (m, 2H), 7.43-7.36 (m, 1H), 7.27 (s, 1H), 6.84-6.80 (m, 3H), 5.48-5.38 (m, 1H), 4.58-4.46 (m, 1H), 4.13 (dd, J=8.0, 11.6 Hz, 1H), 3.93 (s, 3H), 3.87 (s, 3H), 3.78 (s, 2H), 3.02-2.77 (m, 3H), 2.23-2.12 (m, 2H), 1.82-1.49 (m, 4H)

### Example A254—Preparation of Compound 257. (S)-3-(2-((4-(6-((4-Chloro-2-fluorobenzyl)oxy)pyridin-2-yl)piperidin-1-yl)methyl)-4-methoxy-1-(oxetan-2-ylmethyl)-1H-benzo[d]imidazol-6-yl) propiolic acid (Compound 257) (2)

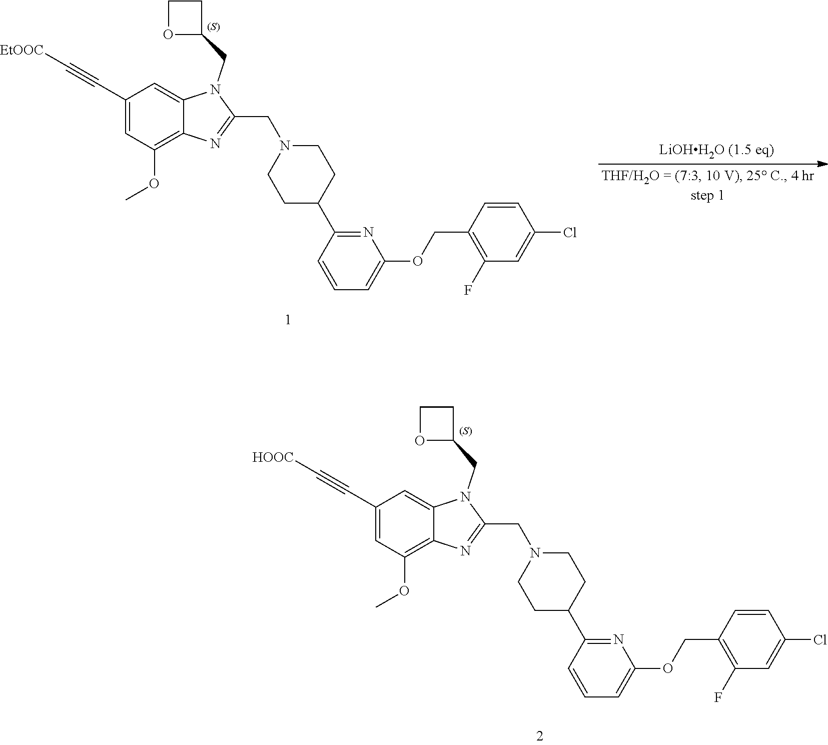

### General Procedure for Preparation of Compound 257 (2):

To a solution of 1 (30 mg, 46.36 μmol, 1 eq) in THF (0.21 mL) and H₂O (0.09 mL) was added LiOH·H₂O (2.92 mg, 69.54 μmol, 1.5 eq). The mixture was stirred at 25° C. for 4 hr. LC-MS showed 1 was consumed completely and desired mass was detected. The reaction mixture was filtered. The filtrate was purified by prep-HPLC (neutral condition; column: Waters Xbridge BEH C18 100*30 mm*10 um; mobile phase: [H₂O (10 mM NH₄HCO₃)-ACN]; gradient: 10%-70% B over 8.0 min) to obtain a crude product, which was further purified by prep-HPLC (neutral condition; column: Waters Xbridge BEH C18 100*30 mm*10 um; mobile phase: [H₂O (10 mM NH₄HCO₃)-ACN]; gradient: 40%-70% B over 8.0 min). (S)-3-(2-((4-(6-((4-Chloro-2-fluorobenzyl)oxy)pyridin-2-yl)piperidin-1-yl)methyl)-4-methoxy-1-(oxetan-2-ylmethyl)-1H-benzo[d]imidazol-6-yl) propiolic acid (7.49 mg, 20.66 μmol, 19.43% yield) was obtained as a white solid.

LCMS: RT=2.860 min, MS cal.: 618.2, [M+H]⁺=619.3

HPLC: RT=11.220 min, purity: 97.57%

¹H NMR (400 MHz, METHANOL-d₄) δ=7.61 (dd, J=7.2, 8.0 Hz, 1H), 7.49 (t, J=8.0 Hz, 1H), 7.44 (d, J=1.2 Hz, 1H), 7.34-7.11 (m, 2H), 6.92 (d, J=0.8 Hz, 1H), 6.87 (d, J=7.2 Hz, 1H), 6.68 (d, J=8.0 Hz, 1H), 5.43 (s, 2H), 5.22 (dq, J=2.4, 7.2 Hz, 1H), 4.78-4.70 (m, 1H), 4.70-4.58 (m, 2H), 4.51-4.38 (m, 3H), 4.00 (s, 3H), 3.61-3.44 (m, 2H), 2.99 (br s, 2H), 2.93-2.86 (m, 1H), 2.84-2.74 (m, 1H), 2.59-2.42 (m, 1H), 2.05 (br s, 4H).

### Example A255—Preparation of Compound 256. 2-((4-((6-((4-chloro-2-fluorophenoxy)methyl)pyridin-2-yl)oxy)piperidin-1-yl)methyl)-4-ethoxy-1-methyl-1H-benzo[d]imidazole-6-carboxylic acid (Compound 256) (5)

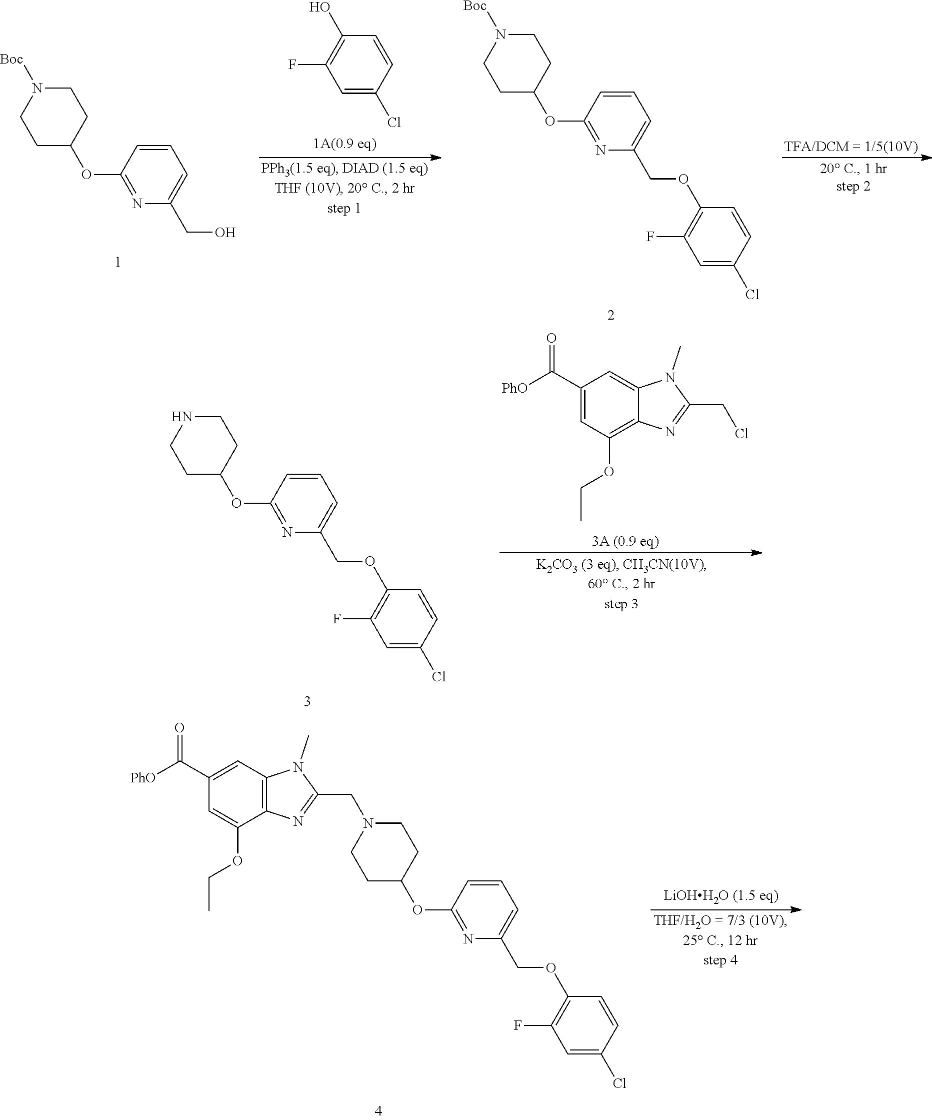 [SMILES: CC(C)(C)OC(=O)N1CCC(Oc2cccc(CO)n2)CC1 ; Oc1ccc(Cl)cc1F ; CC(C)(C)OC(=O)N1CCC(Oc2cccc(COc3ccc(Cl)cc3F)n2)CC1 ; Fc1cc(Cl)ccc1OCc1cccc(OC2CCNCC2)n1 ; CCOc1cc(C(=O)Oc2ccccc2)cc2c1nc(CCl)n2C ; CCOc1cc(C(=O)Oc2ccccc2)cc2c1nc(CN1CCC(Oc3cccc(COc4ccc(Cl)cc4F)n3)CC1)n2C]

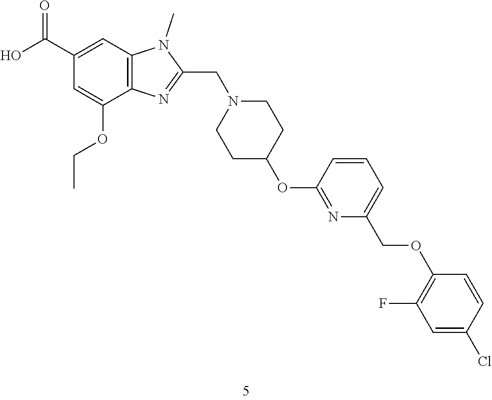 [SMILES: CCOc1cc(C(=O)O)cc2c1nc(CN1CCC(Oc3cccc(COc4ccc(Cl)cc4F)n3)CC1)n2C]

### General Procedure for Preparation of Intermediate 2:

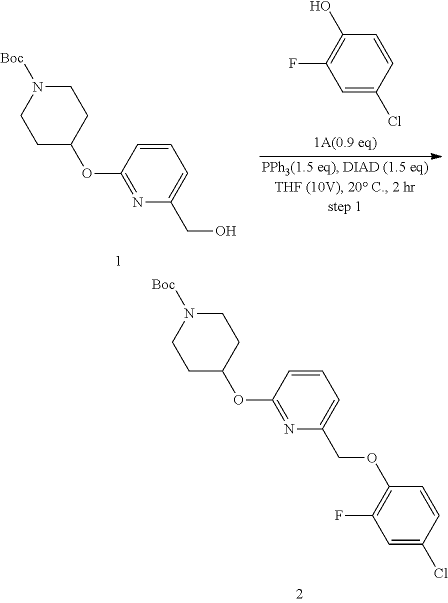 [SMILES: CC(C)(C)OC(=O)N1CCC(Oc2cccc(CO)n2)CC1 ; Oc1ccc(Cl)cc1F ; CC(C)(C)OC(=O)N1CCC(Oc2cccc(COc3ccc(Cl)cc3F)n2)CC1]

To a solution of 1 (300 mg, 972.85 μmol, 1 eq), 1A (128.31 mg, 875.57 μmol, 93.11 μL, 0.9 eq) in THF (1 mL) and PPh₃ (382.75 mg, 1.46 mmol, 1.5 eq) in THF (1 mL) was added DIAD (295.08 mg, 1.46 mmol, 282.92 μL, 1.5 eq) in THF (1 mL) at 20° C. The mixture was stirred at 20° C. for 2 hr. LCMS (product: RT=1.721 min) showed the starting material was consumed completely. The reaction mixture was quenched by addition H₂O 5 mL at 20° C., and then extracted with EtOAc (3 mL*3). The combined organic layers were washed with brine (3 mL*2), dried over Na₂SO₄, filtered and concentrated under reduced pressure to give a residue. The residue was purified by column chromatography (SiO₂, PE/EtOAc=1/1 to 0/1). 2 (300 mg, 686.65 μmol, 70.58% yield) was obtained as a colourless oil.

¹H NMR (400 MHz, DMSO-d6) δ=7.73 (t, J=7.8 Hz, 1H), 7.47 (dd, J=2.4, 11.2 Hz, 1H), 7.34-7.16 (m, 2H), 7.09-7.02 (m, 1H), 6.73 (d, J=8.4 Hz, 1H), 5.19 (s, 2H), 5.13-5.02 (m, 1H), 3.67 (td, J=4.8, 13.3 Hz, 2H), 3.11 (br s, 2H), 1.88 (td, J=4.4, 8.6 Hz, 2H), 1.57-1.44 (m, 2H), 1.40 (s, 9H)

### General Procedure for Preparation of Intermediate 3:

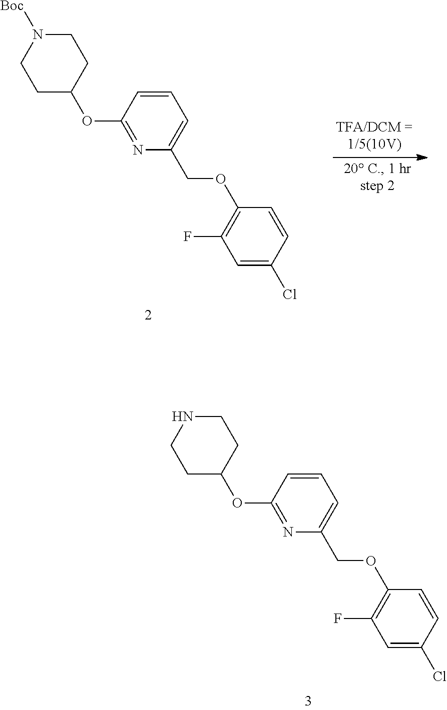 [SMILES: CC(C)(C)OC(=O)N1CCC(Oc2cccc(COc3ccc(Cl)cc3F)n2)CC1 ; Fc1cc(Cl)ccc1OCc1cccc(OC2CCNCC2)n1]

To a solution of 2 (250 mg, 572.21 μmol, 1 eq) in DCM (2.5 mL) was added TFA (0.5 mL). The mixture was stirred at 20° C. for 2 hr. LCMS (product: RT=0.416 min) showed the starting material was consumed completely. The reaction mixture was added to sat. Na₂CO₃ adjusting to pH=9-10 then extracted with EtOAc (3 mL*3). The combined organic layers were washed with brine (3 mL*2), dried over Na₂SO₄, filtered, and concentrated under reduced pressure to give a residue. 3 (200 mg, crude) was obtained as a colourless oil.

### General Procedure for Preparation of Intermediate 4:

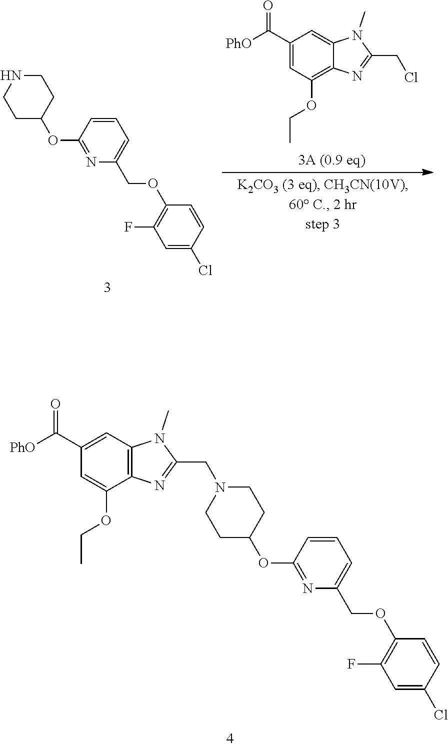 [SMILES: Fc1cc(Cl)ccc1OCc1cccc(OC2CCNCC2)n1 ; CCOc1cc(C(=O)Oc2ccccc2)cc2c1nc(CCl)n2C ; CCOc1cc(C(=O)Oc2ccccc2)cc2c1nc(CN1CCC(Oc3cccc(COc4ccc(Cl)cc4F)n3)CC1)n2C]

To a solution of 3 (180 mg, 534.46 μmol, 1 eq) and 4 (165.85 mg, 481.02 μmol, 0.9 eq) in CH₃CN (1.8 mL) was added K₂CO₃ (221.60 mg, 1.60 mmol, 3 eq) at 20° C. The mixture was stirred at 60° C. for 2 hr. TLC (PE/EtOAc=1/1, product Rf=0.20) showed the starting material was consumed completely. The reaction mixture cooled down to 20° C. and quenched by addition H₂O 5 mL at 20° C., and then extracted with EtOAc (3 mL*3). The combined organic layers were washed with brine (3 mL*2), dried over Na₂SO₄, filtered and concentrated under reduced pressure to give a residue. The residue was purified by column chromatography (SiO₂, PE/EtOAc=1/1 to 0/1). 4 (220 mg, 341.02 μmol, 63.81% yield) was obtained as a white solid.

¹H NMR (400 MHz, DMSO-d6) δ=8.02 (d, J=1.2 Hz, 1H), 7.71 (t, J=7.6 Hz, 1H), 7.53-7.42 (m, 3H), 7.38 (d, J=0.8 Hz, 1H), 7.35-7.24 (m, 4H), 7.21-7.15 (m, 1H), 7.04 (d, J=7.2 Hz, 1H), 6.72 (d, J=8.4 Hz, 1H), 5.18 (s, 2H), 4.99-4.89 (m, 1H), 4.30 (q, J=7.2 Hz, 2H), 3.94 (s, 3H), 3.84 (s, 2H), 2.82-2.68 (m, 2H), 2.31 (t, J=9.6 Hz, 2H), 1.97-1.87 (m, 2H), 1.70-1.53 (m, 2H), 1.43 (t, J=7.2 Hz, 3H)

### General Procedure for Preparation of Compound 256 (5):

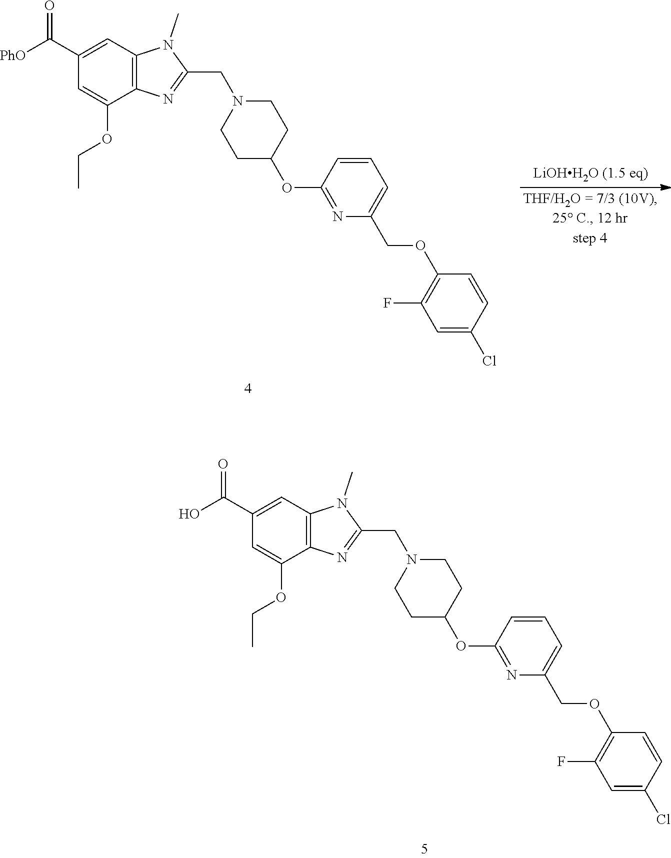 [SMILES: CCOc1cc(C(=O)Oc2ccccc2)cc2c1nc(CN1CCC(Oc3cccc(COc4ccc(Cl)cc4F)n3)CC1)n2C ; CCOc1cc(C(=O)O)cc2c1nc(CN1CCC(Oc3cccc(COc4ccc(Cl)cc4F)n3)CC1)n2C]

To a solution of 4 (200 mg, 310.02 μmol, 1 eq) in THF (1.4 mL) was added LiOH·H₂O (19.51 mg, 465.03 μmol, 1.5 eq) in H₂O (0.6 mL) at 25° C. The mixture was stirred at 25° C. for 12 hr. LCMS (ET64759-791-P1A1, product: RT=1.163 min) showed the starting material was consumed completely. The reaction mixture was filtered. The filtrate was purified by prep-HPLC (column: Waters Xbridge Prep OBD C18 150*40 mm*10 um; mobile phase: [H₂O (10 mM NH₄HCO₃)—CH₃CN]; gradient: 15%-65% B over 8.0 min). 2-((4-((6-((4-chloro-2-fluorophenoxy)methyl)pyridin-2-yl)oxy)piperidin-1-yl)methyl)-4-ethoxy-1-methyl-1H-benzo[d]imidazole-6-carboxylic acid (29.65 mg, 51.64 μmol, 16.66% yield, 99.11% purity) was obtained as a white solid.

LCMS: RT=2.700 min, MS cal.: 568.2, [M+H]⁺=569.2

HPLC: RT=11.673 min

¹H NMR (400 MHz, METHANOL-d4) δ=7.80 (d, J=1.2 Hz, 1H), 7.69-7.60 (m, 1H), 7.43 (s, 1H), 7.19 (dd, J=2.4, 11.0 Hz, 1H), 7.15-7.01 (m, 3H), 6.66 (d, J=8.4 Hz, 1H), 5.12 (s, 2H), 5.03 (td, J=4.4, 8.0 Hz, 1H), 4.28 (q, J=7.2 Hz, 2H), 3.95 (s, 3H), 3.87 (s, 2H), 2.87-2.74 (m, 2H), 2.43 (br t, J=9.2 Hz, 2H), 2.00 (br d, J=12.0 Hz, 2H), 1.83-1.69 (m, 2H), 1.52 (t, J=7.2 Hz, 3H)

### Example A256—Preparation of Compound 380. 2-((4-(6-((4-Chloro-2-fluorobenzyl)oxy)pyridin-2-yl)piperidin-1-yl)methyl)-4-methoxy-1-methyl-1H-imidazo[4,5-c]pyridine-6-carboxylic acid (Compound 380) (7)

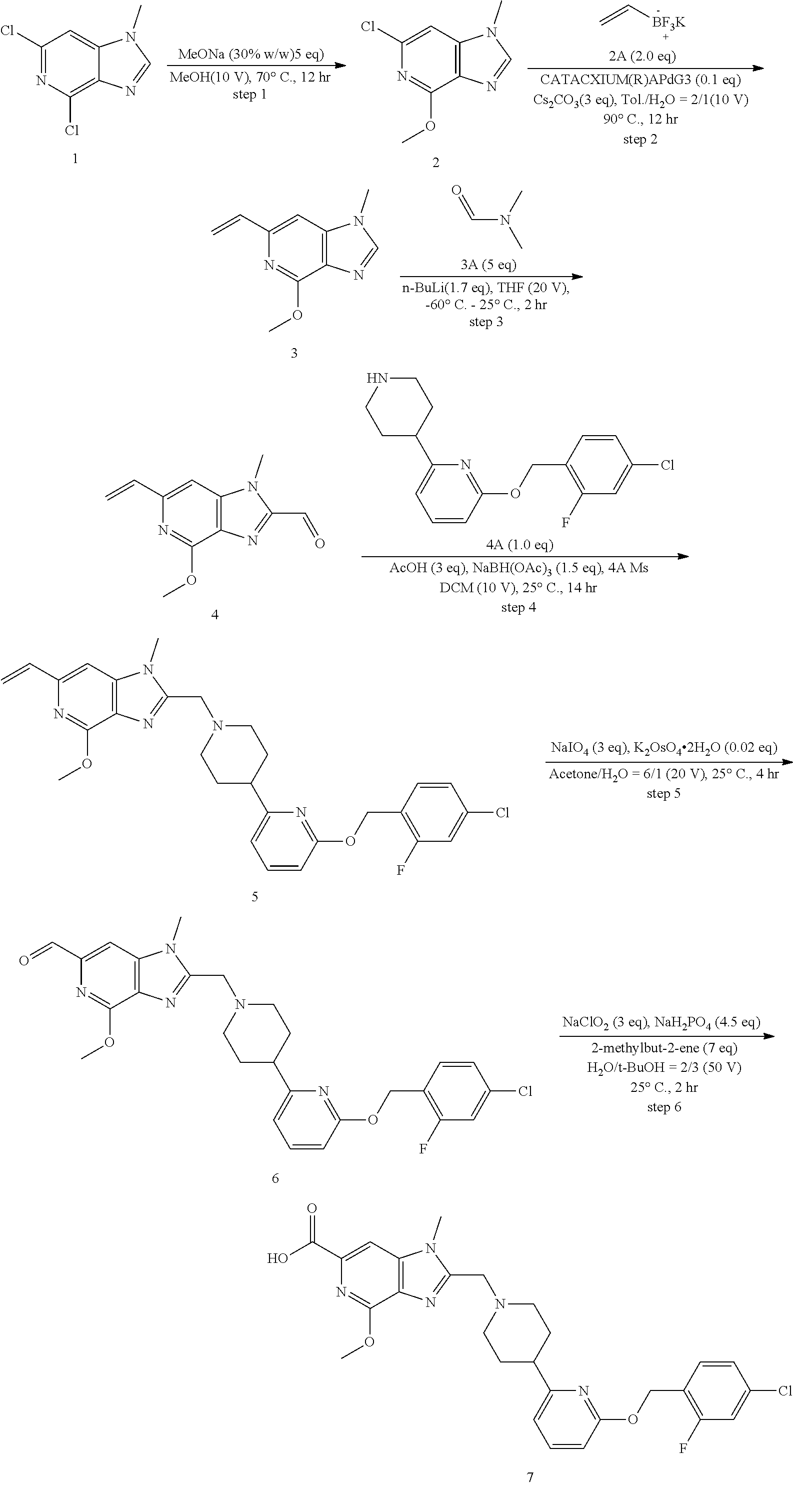

### General Procedure for Preparation of Intermediate 2:

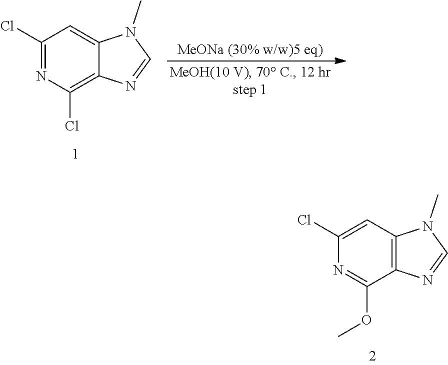

To a solution of 1 (8.5 g, 42.07 mmol, 1 eq) in MeOH (85 mL) was added MeONa (37.88 g, 210.35 mmol, 30% purity, 5 eq) at 25° C. The mixture was stirred at 70° C. for 8 hr. TLC (PE/EtOAc=0/1, Rf=0.54) indicated 1 was consumed completely and one new spot formed. The reaction was clean according to TLC. The mixture was extracted by EtOAc (100 mL*3). Then organic phase was combined and washed by H₂O (100 mL), aq. brine (200 mL), dried with

Na₂SO₄, filtered. The organic phase was concentrated under reduced pressure at 45° C. to give a residue. The residue was purified by column chromatography (SiO₂, PE/EtOAc=10/1 to 0/1). 2 (7.9 g, 39.98 mmol, 95.02% yield) was a pink solid.

¹H NMR (400 MHz, CHLOROFORM-d) δ=7.74 (s, 1H), 6.93 (s, 1H), 4.09 (s, 3H), 3.75 (s, 3H).

### General Procedure for Preparation of Intermediate 3:

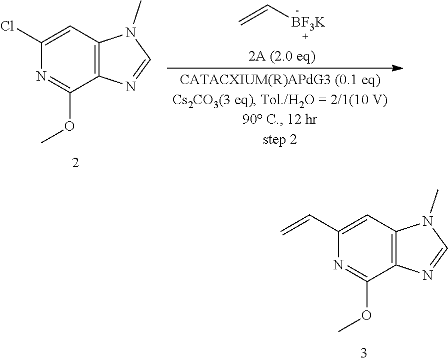

To a solution of 2 (5 g, 25.30 mmol, 1 eq) in Tol. (50 mL) and H₂O (25 mL) was added 2A (8.71 g, 50.60 mmol, 2 eq, K*), Cs₂CO₃ (24.73 g, 75.90 mmol, 3 eq), and CATACXIUM(R)APdG₃ (1.84 g, 2.53 mmol, 0.1 eq). The mixture was stirred at 90° C. for 12 hr. LC-MS showed 2 was consumed completely and one main peak with desired mass was detected. The residue was poured into water (100 mL). The aqueous phase was extracted with EtOAc (50 mL*3). The combined organic phase was washed with brine (50 mL*2), dried with anhydrous Na₂SO₄, filtered and concentrated in vacuum. The residue was purified by column chromatography (SiO₂, PE/EtOAc=10/1 to 0/1). 3 (3 g, 15.86 mmol, 62.67% yield) was obtained as a yellow solid.

LCMS: Rt=0.901 min, MS cal.: 189.09, [M+H]⁺=190.3

¹H NMR (400 MHz, CHLOROFORM-d) δ=7.78-7.74 (m, 1H), 7.00 (s, 1H), 6.88 (s, 1H), 6.84-6.74 (m, 1H), 6.37-6.29 (m, 1H), 5.42-5.36 (m, 1H), 4.20-4.15 (m, 2H), 4.16-4.13 (m, 1H), 3.83-3.79 (m, 4H).

### General Procedure for Preparation of Intermediate 4:

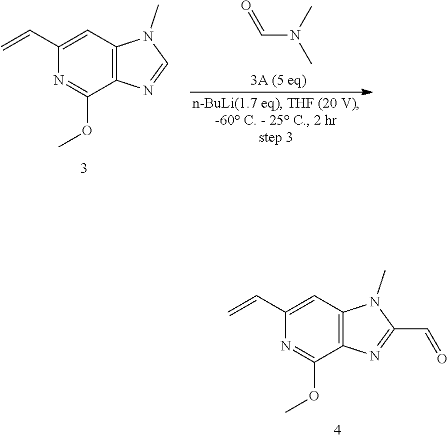

The reactions were carried out in parallels as 4 batches. Equip a 100 mL three-necked round bottom flask, addition funnel and thermometer, N₂ balloon. THF (22 mL) was charged to the three-necked round bottom flask, then 3 (1.1 g, 5.81 mmol, 1 eq) was added to the mixture at 25° C. At −60° C. (inner temperature), n-BuLi (2.5 M, 3.95 mL, 1.7 eq) was added dropwise to the reaction mixture at −60° C. within 0.5 h. Then was added DMF (2.12 g, 29.07 mmol, 2.24 mL, 5 eq) at −60° C. After the addition, the mixture was stirred at −60° C. for 2 hr. LC-MS showed 3 was consumed completely and one main peak with desired mass was detected. Four reactions were combined for the workup. After 2.5 hr, the reaction mixture was added to 200 mL H₂O at 0° C. within 30 min. The mixture was extracted by EtOAc (100 mL*3). Then the organic phase was combined and washed by H₂O (100 mL), aq. brine (100 mL), dried with Na₂SO₄, filtered. The organic phase was concentrated under reduced pressure at 45° C. to give a residue. The residue was purified by column chromatography (SiO₂, PE/EtOAc=10/1 to 0/1). 4 (1.57 g, 7.23 mmol, 31.08% yield) was obtained as a yellow solid.

LCMS: Rt=1.093 min, MS cal.: 217.09, [M+H]⁺=218.0

¹H NMR (400 MHz, CHLOROFORM-d) δ=10.10-9.99 (m, 1H), 6.91-6.85 (m, 1H), 6.85-6.75 (m, 1H), 6.49-6.37 (m, 1H), 5.53-5.44 (m, 1H), 4.26-4.22 (m, 3H), 4.13-4.10 (m, 3H)

### General Procedure for Preparation of Intermediate 5:

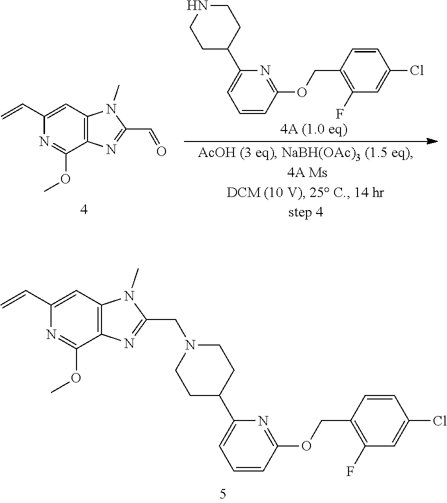 [SMILES: C=Cc1cc2c(nc(C=O)n2C)c(OC)n1 ; Fc1cc(Cl)ccc1COc1cccc(C2CCNCC2)n1 ; C=Cc1cc2c(nc(CN3CCC(c4cccc(OCc5ccc(Cl)cc5F)n4)CC3)n2C)c(OC)n1]

Equip a 25 mL three-necked round bottom flask, addition funnel and thermometer, N₂ balloon. DCM (7 mL) was charged to the three-necked round bottom flask, then 4 (700 mg, 3.22 mmol, 1 eq), AcOH (580.55 mg, 9.67 mmol, 553.43 μL, 3 eq), 4A MS (3.22 mmol, 1 eq) and 4A (1.03 g, 3.22 mmol, 1 eq) was added to the mixture at 25° C. for 12 h. At 25° C. (inner temperature), NaBH(OAc)₃ (1.02 g, 4.83 mmol, 1.5 eq) was added dropwise to the reaction mixture at 25° C. After the addition, the mixture was stirred at 25° C. for 2 hr. LC-MS showed 4 was consumed completely and one main peak with desired mass was detected. After 2 hr, the reaction mixture was added to H₂O (50 mL) at 0° C. The mixture was extracted by DCM (50 mL*3). Then the organic phase was combined and washed by H₂O (50 mL), brine (50 mL), dried over Na₂SO₄, filtered. The organic phase was concentrated under reduced pressure at 40° C. to give a residue. The reaction was combined with ET77627-495 to purification. The residue was purified by column chromatography (SiO₂, PE/EtOAc=10/1 to 0/1). 5 (1.3 g, 2.49 mmol, 77.28% yield) was obtained as a yellow solid.

LCMS: Rt=1.769 min, MS cal.: 521.20/523.20, [M+H]⁺=522.1/524.1

¹H NMR (400 MHz, CHLOROFORM-d) δ=7.49 (t, J=7.6 Hz, 1H), 7.44 (t, J=8.0 Hz, 1H), 7.14-7.07 (m, 2H), 6.87 (s, 1H), 6.79 (dd, J=8.0, 17.2 Hz, 1H), 6.72 (d, J=7.2 Hz, 1H), 6.60 (d, J=8.4 Hz, 1H), 6.31 (dd, J=2.0, 17.2 Hz, 1H), 5.41 (s, 2H), 5.37 (dd, J=1.6, 10.4 Hz, 1H), 4.17 (s, 3H), 3.90 (s, 3H), 3.87-3.82 (m, 2H), 2.97 (br d, J=11.6 Hz, 2H), 2.60 (s, 1H), 2.28 (dt, J=2.4, 11.6 Hz, 2H), 1.94-1.75 (m, 4H).

### General Procedure for Preparation of Intermediate 4:

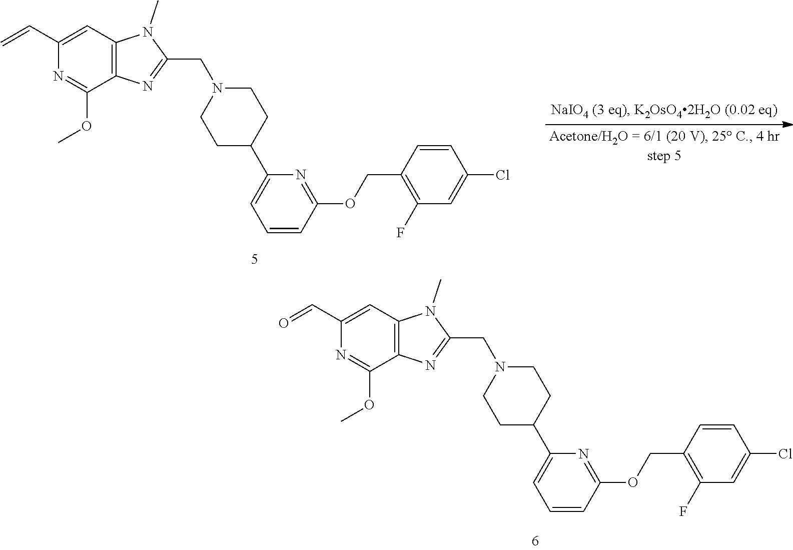 [SMILES: C=Cc1cc2c(nc(CN3CCC(c4cccc(OCc5ccc(Cl)cc5F)n4)CC3)n2C)c(OC)n1 ; COc1nc(C=O)cc2c1nc(CN1CCC(c3cccc(OCc4ccc(Cl)cc4F)n3)CC1)n2C]

The reactions were carried out in parallel as 2 batches. Equip a 25 mL three-necked round bottom flask, addition funnel and thermometer, N₂ balloon. Acetone (2 mL) and H₂O (1.3 mL) were charged to the three-necked round bottom flask, then 5 (200 mg, 383.13 μmol, 1 eq) was added to the mixture at 25° C. At 25° C. (inner temperature) NaIO₄ (245.85 mg, 1.15 mmol, 63.69 μL, 3 eq) and K₂Os₄·H₂O (2.82 mg, 7.66 μmol, 0.02 eq) was added in portions to the reaction mixture at 25° C. After the addition, the mixture was stirred at 25° C. for 4 hr. LC-MS showed 5 was consumed completely and one main peak with desired mass was detected. Two reactions were combined for workup. After 4 hr, the reaction mixture was added to Na₂S₂O₃·5H₂O (8.13 g, 32.76 mmol, 8.05 mL, 10 eq) in H₂O (25 mL). The mixture was adjusted to pH=3˜4 by 1M FA aq. The mixture was extracted by EtOAc (80 mL*3). The combined organic layers were washed with brine (50 mL*3), dried over Na₂SO₄, filtered and concentrated under reduced pressure to give a residue. Two reactions were combined to purify. The residue was purified by column chromatography (SiO₂, PE/EtOAc=10/1 to 1/2). 6 (0.11 g, 209.93 μmol, 27.40% yield) was obtained as a yellow solid.

LCMS: Rt=1.631 min, MS cal.: 523.18/525.18, [M+H]⁺=524.1/525.1

¹H NMR (400 MHz, CHLOROFORM-d) δ=10.06 (s, 1H), 7.78 (s, 1H), 7.50 (t, J=7.6 Hz, 1H), 7.44 (br t, J=7.6 Hz, 1H), 7.20-7.02 (m, 2H), 6.73 (d, J=7.2 Hz, 1H), 6.61 (d, J=8.0 Hz, 1H), 5.40 (s, 2H), 4.24 (s, 3H), 4.07-3.96 (m, 3H), 3.90 (br s, 2H), 3.09-2.87 (m, 2H), 2.68-2.53 (m, 1H), 2.30 (br s, 2H), 1.87 (br s, 4H).

### General Procedure for Preparation of Compound 380 (7):

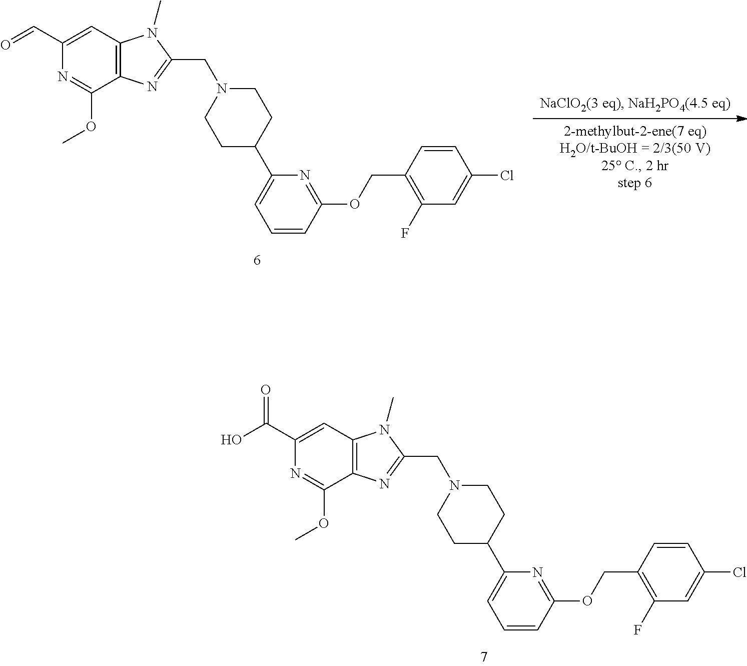

The reactions were carried out in parallel as 5 batches. Equip a 25 mL three-necked round bottom flask, addition funnel and thermometer, N₂ balloon. t-BuOH (0.6 mL) and H₂O (0.4 mL) was charged to the three-necked round bottom flask, then 6 (20 mg, 38.17 μmol, 1 eq), NaH₂PO₄ (20.61 mg, 171.76 μmol, 4.5 eq) and 2-methylbut-2-ene (18.74 mg, 267.18 μmol, 28.31 μL, 7 eq) was added to the mixture at 25° C. At 25° C. (inner temperature), NaClO₄ (10.36 mg, 114.51 μmol, 3 eq) was added in portions to the reaction mixture at 25° C. After the addition, the mixture was stirred at 25° C. for 2 hr. LC-MS showed 6 was consumed completely and one main peak with desired mass was detected. Three reactions were combined for work-up. The reaction was poured into Na₂S₂O₃. The reaction mixture was diluted with H₂O 10 mL and extracted with EtOAc (10 mL*3). The combined organic layers were washed with brine (10 mL*2), dried over Na₂SO₄, filtered, and concentrated under reduced pressure to give a residue. The residue was purified by prep-HPLC (column: Waters Xbridge BEH C18 100*30 mm*10 um; mobile phase: [H₂O (10 mM NH₄HCO₃)-ACN]; gradient: 30%-70% B over 8.0 min). 2-((4-(6-((4-Chloro-2-fluorobenzyl)oxy)pyridin-2-yl)piperidin-1-yl)methyl)-4-methoxy-1-methyl-1H-imidazo[4,5-c]pyridine-6-carboxylic acid (6.48 mg, 12.00 μmol, 6.29% yield) was obtained as a white solid.

HPLC: Rt=11.044 min, purity 90.28%

LCMS: Rt=2.703 min, MS cal.: 539.17/541.17, [M+H]⁺=540.2/541.2

¹H NMR (400 MHz, DMSO-d6) δ=7.99 (s, 1H), 7.62 (t, J=7.6 Hz, 1H), 7.55 (t, J=8.0 Hz, 1H), 7.45 (dd, J=2.0, 10.0 Hz, 1H), 7.28 (dd, J=2.0, 8.4 Hz, 1H), 6.86 (d, J=7.2 Hz, 1H), 6.66 (d, J=8.4 Hz, 1H), 5.36 (s, 2H), 4.04 (s, 3H), 3.93 (s, 3H), 3.82 (s, 2H), 2.92 (br d, J=11.2 Hz, 2H), 2.57 (br d, J=12.4 Hz, 1H), 2.24-2.17 (m, 2H), 1.84-1.69 (m, 4H).

### Example A257—Preparation of Compound 381. 2-((4-(6-((4-Chloro-2-fluorophenyl)methoxy-d2)pyridin-2-yl)piperidin-1-yl)methyl-d2)-4-(ethoxy-1,1-d2)-1-methyl-1H-benzo[d]imidazole-6-carboxylic acid (Compound 381) (9)

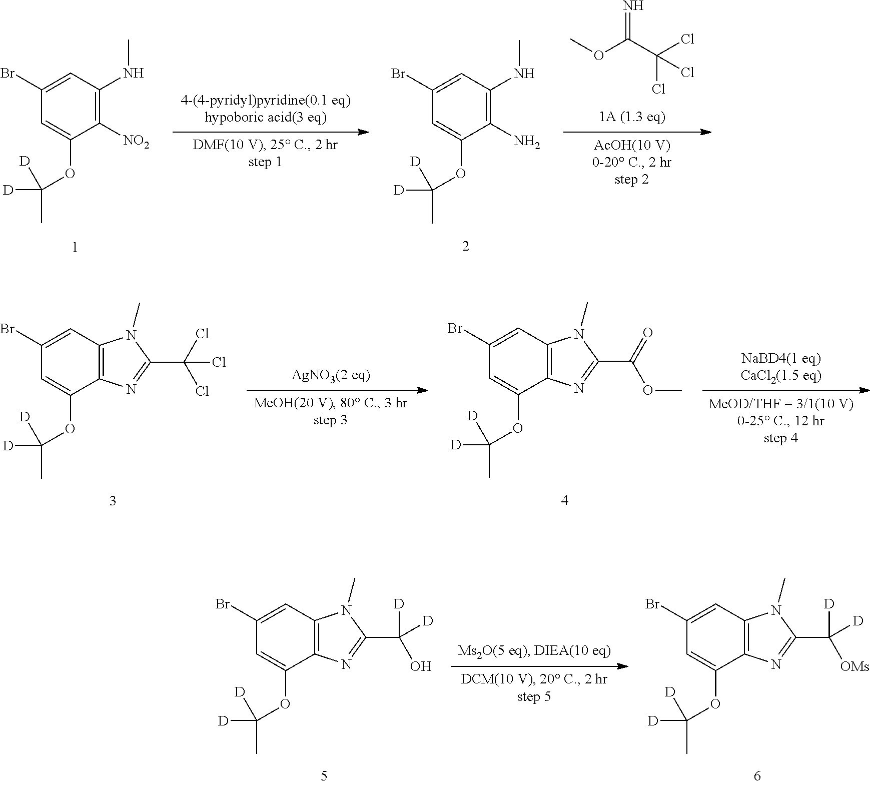

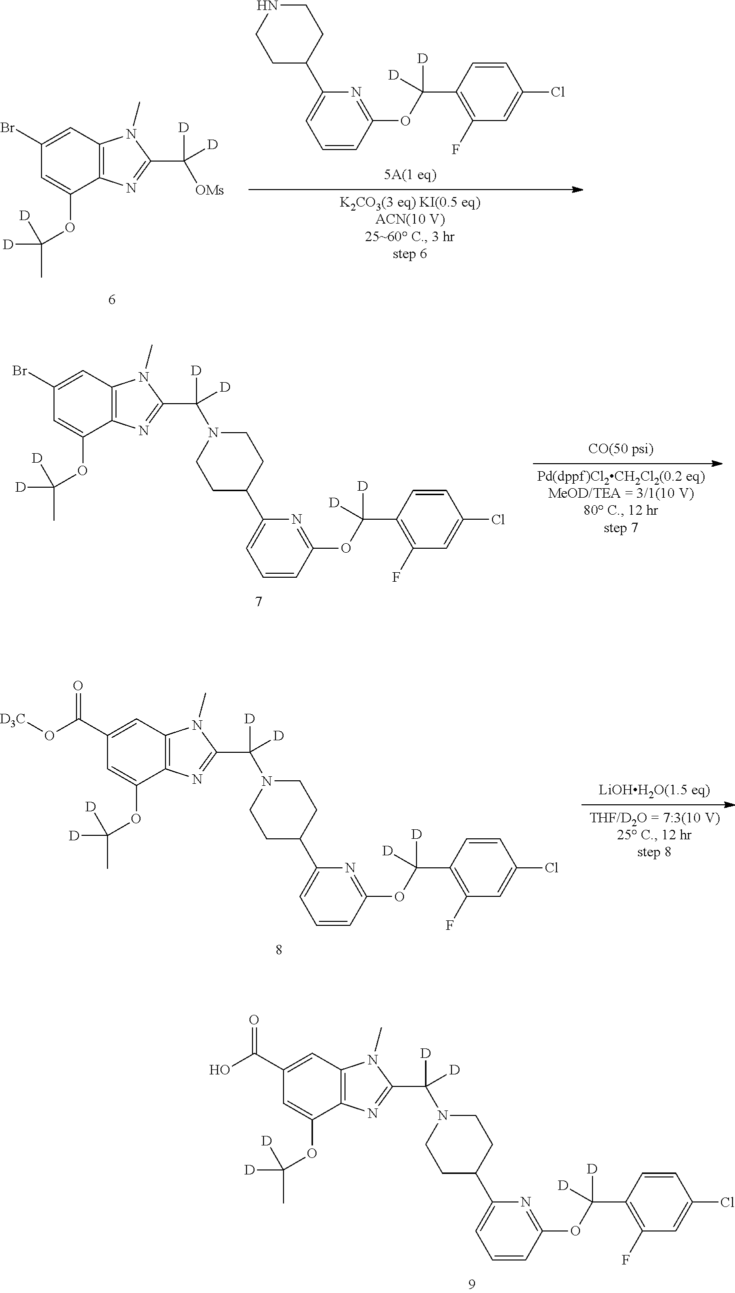

### General Procedure for Preparation of Intermediate 2:

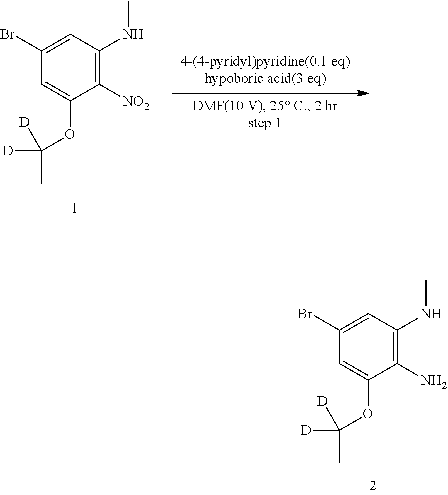 [SMILES: [2H]C([2H])(C)Oc1cc(Br)cc(NC)c1[N+](=O)[O-] ; "4-(4-pyridyl)pyridine(0.1 eq)hypoboric acid(3 eq)" ; "DMF(10 V), 25° C., 2 hrstep 1" ; [2H]C([2H])(C)Oc1cc(Br)cc(NC)c1N]

To a solution of 1 (1 g, 3.61 mmol, 1 eq) in DMF (10 mL) was added 4-(4-pyridyl)pyridine (56.36 mg, 360.87 μmol, 0.1 eq) and hypoboric acid (970.55 mg, 10.83 mmol, 3 eq). The mixture was stirred at 25° C. for 2 hr. LCMS showed 1 was consumed completely and one main peak with desired m/z was detected. TLC (Petroleum ether:Ethyl acetate=3:1) indicated 1 was consumed completely and one new spot formed. The reaction was clean according to TLC. The mixture was diluted with H₂O 10 mL, extracted by Ethyl acetate (15 mL*3). Then the organic phase was washed by brine (5 mL*2). The organic were dried with Na₂SO₄, filtered. The organic phase was concentrated under reduced pressure at 45° C. to give a residue. 2 (1 g, crude) was obtained as a yellow solid.

LCMS: RT=1.197 min, MS cal.: 246.03, 248.03, [M+H]⁻=246.8

### General Procedure for Preparation of Intermediate 3:

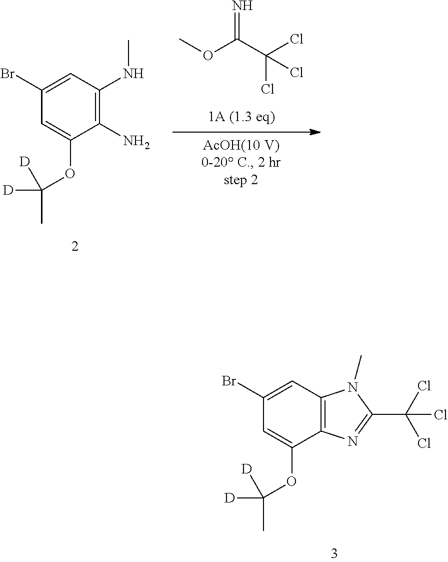

To a solution of 2 (1.68 g, 6.80 mmol, 1 eq) in AcOH (16.8 mL) was added 1A (1.56 g, 8.84 mmol, 1.09 mL, 1.3 eq) at 0° C. The mixture was stirred at 20° C. for 2 hr. LCMS showed 2 was consumed completely and one main peak with desired m/z was detected. TLC (Petroleum ether:Ethyl acetate=3:1) indicated 2 was consumed completely and one new spot formed. The aqueous layer was neutralized by aq. NaHCO₃ aq. at 0° C. to pH around 7-8. The reaction mixture was diluted with H₂O 10 mL, extracted by Ethyl acetate (15 mL*3). Then the organic phase was washed by brine (10 mL*2). The organic was dried over Na₂SO₄, filtered. The organic phase was concentrated under reduced pressure at 45° C. to give a residue. The residue was purified by column chromatography (SiO₂, Petroleum ether/Ethyl acetate=100/0 to 80/1). 3 (1.7 g, 4.13 mmol, 60.77% yield, 91% purity) was obtained as a white solid.

LCMS: RT=1.470 min, MS cal.: 371.92, 373.91, [M+H]⁻=374.8

1H NMR (400 MHz, DMSO-d6) δ=7.58 (d, J=1.2 Hz, 1H), 6.99 (d, J=1.2 Hz, 1H), 4.06 (s, 3H), 1.39 (s, 3H)

### General Procedure for Preparation of Intermediate 4:

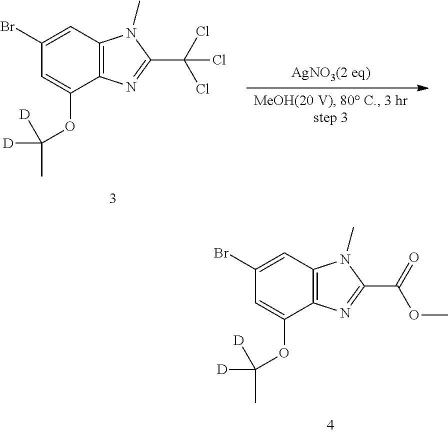 [SMILES: [2H]C([2H])(C)Oc1cc(Br)cc2c1nc(C(Cl)(Cl)Cl)n2C ; "AgNO3(2 eq)" ; "MeOH(20 V), 80° C., 3 hrstep 3" ; [2H]C([2H])(C)Oc1cc(Br)cc2c1nc(C(=O)OC)n2C]

MeOH (10 mL) was charged to the three-necked round bottom flask, then 3 (0.5 g, 1.34 mmol, 1 eq) was added to the mixture at 25° C. At 25° C., AgNO₃ (453.62 mg, 2.67 mmol, 2 eq) was added in portions to the reaction mixture. After the addition, the mixture was stirred at 80 C for 3 hr. LC-MS showed 3 was consumed completely and one main peak with desired m/z was detected. The aqueous layer was neutralized by aq. NaHCO₃ aq. at 0° C. to pH around 7-8. The reaction mixture was filtered, and the filter cake was washed with 1 mL of Ethyl acetate. The filtrate was diluted with H₂O 5 mL, extracted by Ethyl acetate (10 mL*3). Then organic phase was washed by brine (2 mL*2). The organic were dried with Na₂SO₄, filtered. The organic phase was concentrated under reduced pressure at 45° C. to give a residue. 4 (540 mg, crude) was obtained as a brown solid.

LCMS: RT=1.207 min, MS cal.: 314.02, 316.02, [M+H]⁻=314.9

### General Procedure for Preparation of Intermediate 5:

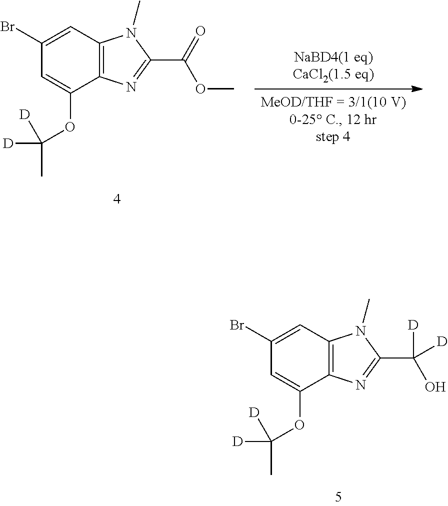

To a solution of 4 (0.6 g, 1.90 mmol, 1 eq) in THF (1.5 mL) and MeOD (4.5 mL) was added CaCl₂) (316.94 mg, 2.86 mmol, 1.5 eq) at 25° C. Then NaBD₄ (72.03 mg, 1.90 mmol, 1 eq) was added in portions at 0° C., then the mixture was stirred at 25° C. for 12 hr. LCMS showed 4 was consumed completely and many main peaks with desired m/z was detected. TLC (Petroleum ether/Ethyl acetate=1:1) indicated 4 was completely and one new spot formed. The reaction was clean according to TLC. The reaction mixture was added to D₂O 5 mL at 0° C. The mixture was diluted with H₂O 10 mL, extracted by Ethyl acetate (15 mL*3). Then the organic phase was washed by brine (5 mL*2). The organic was dried with Na₂SO₄, filtered. The organic phase was concentrated under reduced pressure at 45° C. to give a residue. The residue was triturated with MTBE (10 mL) at 25° C. for 0.5 hr. The mixture was filtered and collected. The filtered cake was concentrated to give the product. 5 (450 mg, 1.37 mmol, 35.97% yield, 88% purity) was obtained as a white solid.

LCMS: RT=1.032 min, MS cal.: 288.04, 290.04, [M+H]⁻=288.9

1H NMR (400 MHz, DMSO-d6) δ=7.38 (s, 1H), 6.82 (s, 1H), 5.52 (s, 1H), 3.77 (s, 3H), 1.35 (s, 3H)

### General Procedure for Preparation of Intermediate 6:

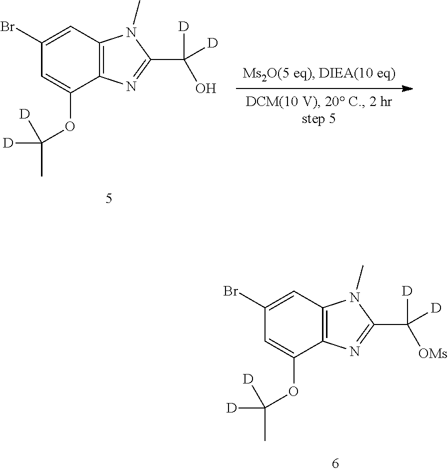 [SMILES: [2H]C([2H])(C)Oc1cc(Br)cc2c1nc(C([2H])([2H])O)n2C ; "DCM(10 V), 20° C., 2 hrstep 5" ; "Ms2O(5 eq), DIEA(10 eq)" ; [2H]C([2H])(C)Oc1cc(Br)cc2c1nc(C([2H])([2H])OS(C)(=O)=O)n2C]

To a solution of 5 (430 mg, 1.49 mmol, 1 eq) in DCM (4.3 mL) was added DIEA (1.92 g, 14.87 mmol, 2.59 mL, 10 eq) and Ms₂O (1.30 g, 7.44 mmol, 5 eq). The mixture was stirred at 20° C. for 2 hr. LCMS showed 5 was consumed completely and one main peak with desired m/z was detected. The mixture was diluted with H₂O 5 mL, extracted by Ethyl acetate (10 mL*3). Then the organic phase was washed by brine (5 mL*2). The organic were dried with Na₂SO₄, filtered. The organic phase was concentrated under reduced pressure at 45° C. to give a residue. 6 (546 mg, crude) was obtained as orange oil.

LCMS: RT=1.166 min, MS cal.: 366.02, 368.02, [M+H]⁺=368.8

### General Procedure for Preparation of Intermediate 7:

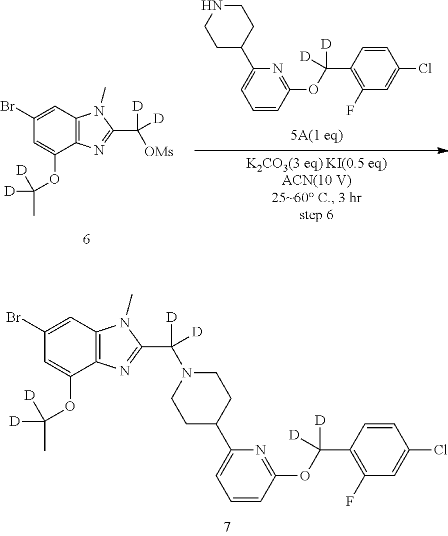

To a solution of 6 (546 mg, 981.23 μmol, 1 eq) in CH₃CN (5.46 mL) was added 5A (316.74 mg, 981.23 μmol, 1 eq) and KI (81.44 mg, 490.62 μmol, 0.5 eq) and K₂CO₃ (406.84 mg, 2.94 mmol, 3 eq) at 25° C. The mixture was stirred at 60° C. for 3 hr. LCMS showed 6 was consumed completely and one main peak with desired m/z was detected. TLC (Petroleum ether:Ethyl acetate=0:1) indicated 6 was consumed completely and one new spot formed. The reaction was clean according to TLC. The mixture was diluted with H₂O 10 mL, extracted by Ethyl acetate (15 mL*3). Then the organic phase was washed by brine (5 mL*2). The organic was dried with Na₂SO₄, filtered. The organic phase was concentrated under reduced pressure at 45° C. to give a residue. The residue was purified by column chromatography (SiO₂, Petroleum ether/Ethyl acetate=100/0 to 1/1). 7 (310 mg, 521.93 μmol, 53.19% yield) was obtained as an orange oil.

LCMS: RT=1.843 min, MS cal.: 592.15, 594.15, [M+H]⁺=595.0

1H NMR (400 MHz, DMSO-d6) δ=7.61 (t, J=8.0 Hz, 1H), 7.55 (t, J=8.4 Hz, 1H), 7.44 (dd, J=2.0, 10.0 Hz, 1H), 7.39 (d, J=1.6 Hz, 1H), 7.28 (dd, J=1.6, 8.4 Hz, 1H), 6.85 (d, J=7.2 Hz, 1H), 6.81 (d, J=1.6 Hz, 1H), 6.66 (d, J=8.4 Hz, 1H), 4.09 (q, J=5.2 Hz, 3H), 3.83 (s, 3H), 3.17 (d, J=5.2 Hz, 8H), 2.89 (d, J=11.2 Hz, 2H), 2.63-2.53 (m, 1H), 2.22-2.11 (m, 2H), 1.82-1.62 (m, 4H), 1.37 (s, 3H)

### General Procedure for Preparation of Intermediate 8:

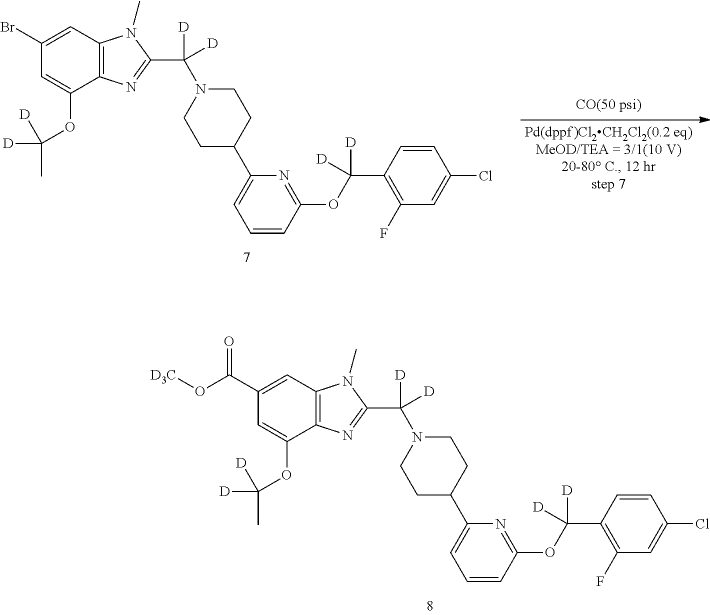

MeOD (3 mL) and TEA (1 mL) was charged to the 35 mL Hydrogenated bottle, then 7 (200 mg, 336.73 μmol, 1 eq) and Pd(dppf)Cl₂·CH₂Cl₂ (55.00 mg, 67.35 μmol, 0.2 eq) was added at 20° C. After the addition, the mixture was degassed and purged with CO 3 times. The mixture was stirred at 80° C. (50 psi) for 12 hr. LCMS showed 7 was consumed completely and one main peak with desired m/z was detected. TLC (Petroleum ether:Ethyl acetate=0:1) indicated 7 was consumed completely and one new spot formed. The reaction was clean according to TLC. The reaction mixture was diluted with H₂O (5 mL), extracted by Ethyl acetate 10 mL (5 mL*2). Then organic phase was combined and washed by brine 10 mL, dried with Na₂SO₄, filtered. The organic phase was concentrated under reduced pressure at 40° C. to give a residue. The crude reaction mixture on notebook page ET74575-614 (0.110 g scale) was combined to ET74575-616 for purification. The residue was purified by column chromatography (SiO₂, Petroleum ether/Ethyl acetate=1/0 to 1/2). 8 (210 mg, 317.13 μmol, 94.18% yield, 87% purity) was obtained as a yellow oil.

LCMS: RT=1.706 min, MS cal.: 575.27, 576.27, [M+H]⁺=576.0

1H NMR (400 MHz, DMSO-d6) δ=7.80 (d, J=1.2 Hz, 1H), 7.64-7.58 (m, 1H), 7.55 (t, J=8.4 Hz, 1H), 7.48-7.40 (m, 1H), 7.30-7.26 (m, 1H), 7.26-7.24 (m, 1H), 6.85 (d, J=7.6 Hz, 1H), 6.66 (d, J=8.4 Hz, 1H), 3.91 (s, 3H), 2.92 (d, J=11.6 Hz, 2H), 2.63-2.53 (m, 1H), 2.19 (t, J=10.8 Hz, 2H), 1.82-1.63 (m, 4H), 1.40 (s, 3H)

### General Procedure for Preparation of Compound 381 (9):

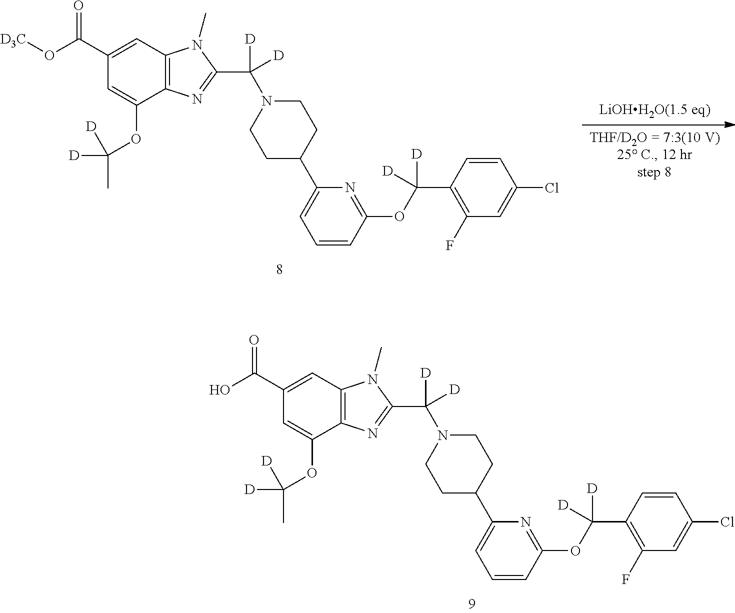

To a solution of 8 (70 mg, 121.51 μmol, 1 eq) in THF (0.49 mL) was added LiOH·H₂O (7.65 mg, 182.26 μmol, 1.5 eq) and D₂O (0.21 mL). The mixture was stirred at 25° C. for 12 hr. LCMS showed 8 was consumed completely and one main peak with desired m/z was detected. The residue was purified by prep-HPLC (column: Waters Xbridge BEH C18 100*30 mm*10 um; mobile phase: [H₂O (10 mM NH₄HCO₃)-ACN]; gradient: 20%-60% B over 8.0 min). 2-((4-(6-((4-Chloro-2-fluorophenyl)methoxy-d2)pyridin-2-yl)piperidin-1-yl)methyl-d2)-4-(ethoxy-1,1-d2)-1-methyl-1H-benzo[d]imidazole-6-carboxylic acid (28.42 mg, 50.84 μmol, 41.84% yield, 100% purity) was obtained as a white solid.

LCMS: Rt=2.765 min, MS cal.: 558.23, 560.23, [M+H]⁺=559.3

HPLC: Rt=11.691 min, purity=100.00%

¹H NMR (400 MHz, DMSO-d₄) δ=7.77 (d, J=1.2 Hz, 1H), 7.65-7.59 (m, 1H), 7.56 (t, J=8.0 Hz, 1H), 7.46 (dd, J=2.0, 10.0 Hz, 1H), 7.29 (dd, J=2.0, 8.4 Hz, 1H), 7.26-7.24 (m, 1H), 6.87 (d, J=7.2 Hz, 1H), 6.67 (d, J=8.0 Hz, 1H), 3.91 (s, 3H), 2.93 (d, J=11.2 Hz, 2H), 2.64-2.55 (m, 1H), 2.24-2.15 (m, 2H), 1.83-1.65 (m, 4H), 1.40 (s, 3H)

### Example A258—Preparation of Compound 382. (S)-5-(2-((4-(6-((4-Chloro-2-fluorobenzyl)oxy)pyridin-2-yl)piperidin-1-yl)methyl)-4-methoxy-1-(oxetan-2-ylmethyl)-1H-benzo[d]imidazol-6-yl) isoxazol-3 (2H)-one (Compound 382) (9)

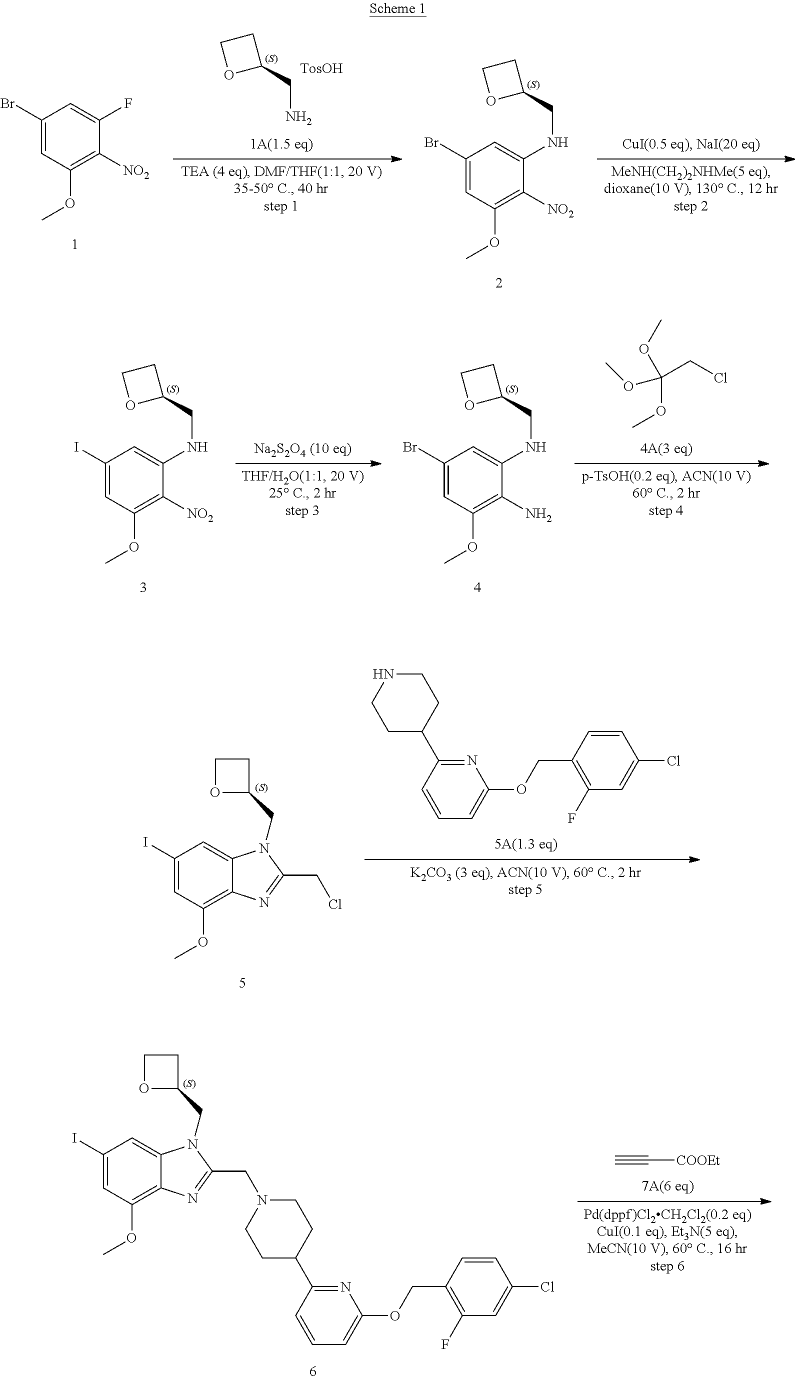

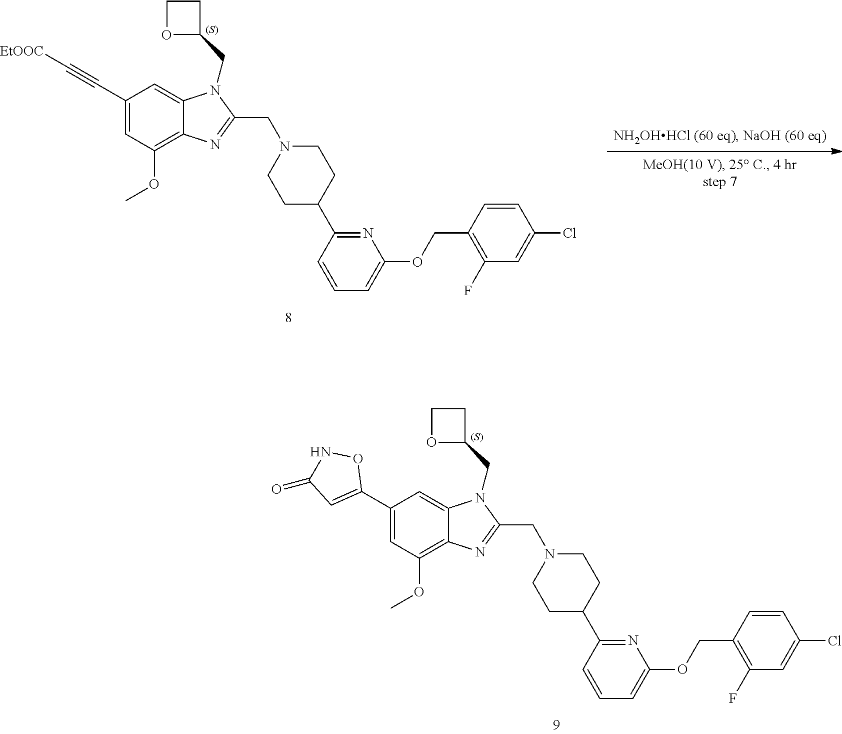

### General Procedure for Preparation of Intermediate 2:

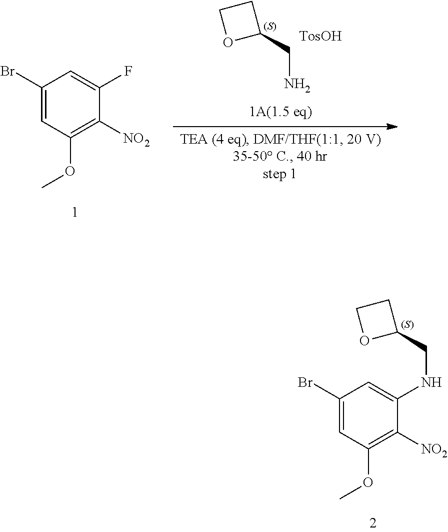

To a solution of 1 (4.5 g, 18.00 mmol, 1 eq) in DMF (45 mL) and THF (45 mL) was added TEA (7.28 g, 71.99 mmol, 10.02 mL, 4 eq) and 1A (7.00 g, 27.00 mmol, 1.5 eq, p-TSA salt) at 25° C. The mixture was stirred at 35° C. for 24 hr and then at 50° C. for 16 hr. LC-MS showed 1 was consumed completely and desired mass was detected. The mixture was poured into H₂O (100 mL) and extracted with ethyl acetate (100 mL*3). The combined organic layers were washed with brine (50 mL*2), dried over Na₂SO₄, filtered and concentrated under vacuum to give a residue. The residue was purified by column chromatography (SiO₂, Petroleum ether/Ethyl acetate=1/0 to 5/1). 2 (5 g, 15.61 mmol, 86.72% yield, 99% purity) was obtained as a yellow oil.

LCMS: RT=0.495 min, MS cal.: 316.0, [M+H]⁺=317.2

### General Procedure for Preparation of Intermediate 3:

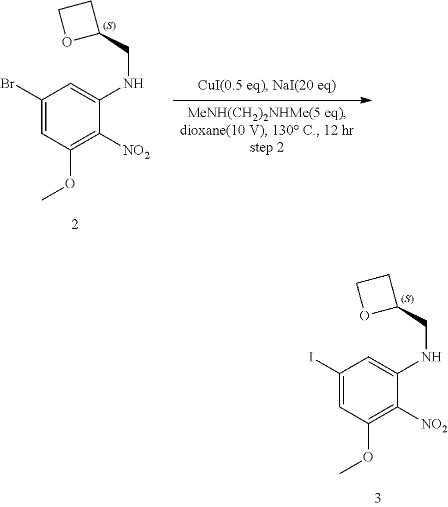

To a mixture of 2 (2.8 g, 8.83 mmol, 1 eq), NaI (26.47 g, 176.58 mmol, 20 eq) and N, N′-dimethylethane-1,2-diamine (1.56 g, 17.66 mmol, 1.90 mL, 5 eq) in dioxane (28 mL) was added CuI (840.74 mg, 4.41 mmol, 0.5 eq) under N₂ atmosphere. The mixture was degassed and purged with N₂ 3 times. The mixture was stirred under N₂ atmosphere at 130° C. for 12 hr. LC-MS showed 2 was consumed completely and desired mass was detected. The residue was poured into H₂O (50 mL) and extracted with ethyl acetate (150 mL*3). The combined organic layers were washed with brine (50 mL), dried over Na₂SO₄, filtered and concentrated under vacuum to give a residue. The residue was purified by column chromatography (SiO₂, Petroleum ether/Ethyl acetate=1/0 to 3/1). 3 (2.4 g, 6.59 mmol, 74.65% yield) was obtained as a yellow solid.

¹H NMR (400 MHz, DMSO-d6) δ=6.98 (d, J=1.2 Hz, 1H), 6.76 (d, J=1.2 Hz, 1H), 6.38 (t, J=5.6 Hz, 1H), 4.88-4.80 (m, 1H), 4.52-4.47 (m, 1H), 4.37 (td, J=6.0, 8.8 Hz, 1H), 3.80 (s, 3H), 3.45-3.35 (m, 2H), 2.66-2.54 (m, 1H), 2.43-2.36 (m, 1H)

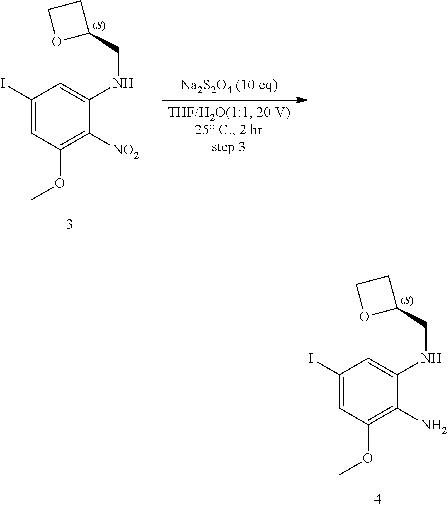

To a solution of 3 (2.4 g, 6.59 mmol, 1 eq) in THF (24 mL) and H₂O (24 mL) was added Na₂S₂O₄ (11.48 g, 65.91 mmol, 10 eq). The mixture was stirred at 25° C. for 12 hr. LC-MS showed 3 was consumed completely and desired mass was detected. The mixture was poured into H₂O (100 mL) and extracted with ethyl acetate (200 mL*3). The combined organic layers were washed with brine (100 mL), dried over Na₂SO₄, filtered, and concentrated under vacuum to give a residue. The residue was purified by column chromatography (SiO₂, Petroleum ether/Ethyl acetate=1/0 to 2/1). 4 (1.4 g, 4.19 mmol, 63.57% yield) was obtained as a yellow solid.

¹H NMR (400 MHz, DMSO-d6) δ=6.57 (d, J=1.6 Hz, 1H), 6.51 (d, J=1.6 Hz, 1H), 4.90-4.82 (m, 1H), 4.77 (t, J=5.6 Hz, 1H), 4.55-4.49 (m, 1H), 4.47-4.41 (m, 1H), 4.26 (s, 2H), 3.72 (s, 3H), 3.31-3.18 (m, 2H), 2.68-2.58 (m, 1H), 2.44-2.38 (m, 1H)

### General Procedure for Preparation of Intermediate 5:

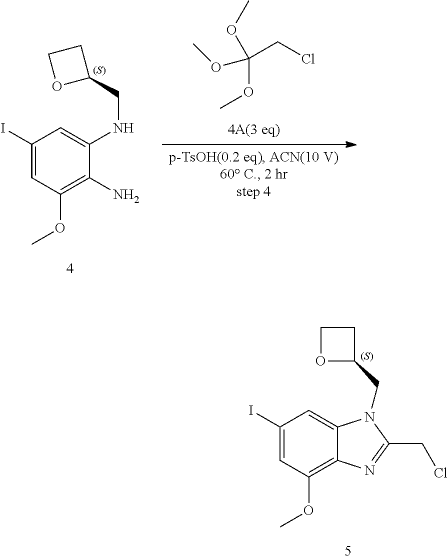

To a solution of 4 (1.4 g, 3.85 mmol, 1 eq) and 4A (1.79 g, 11.56 mmol, 1.56 mL, 3 eq) in ACN (14 mL) was added p-TsOH (132.75 mg, 770.91 μmol, 0.2 eq). The mixture was stirred at 25° C. for 1 hr. LC-MS showed 4 was consumed completely and desired mass was detected. The mixture was filtered, and the filtrate was concentrated under reduced pressure to give a residue. The residue was purified by column chromatography (SiO₂, Petroleum ether/Ethyl acetate=1/0 to 1/1). 5 (1.1 g, 2.80 mmol, 72.69% yield) was obtained as a yellow solid.

¹H NMR (400 MHz, DMSO-d6) δ=7.70 (s, 1H), 7.01 (s, 1H), 5.10-4.99 (m, 3H), 4.65-4.55 (m, 1H), 4.53-4.42 (m, 2H), 4.31 (td, J=6.0, 9.2 Hz, 1H), 3.92 (s, 3H), 2.73-2.59 (m, 1H), 2.39-2.27 (m, 1H)

### General Procedure for Preparation of Intermediate 6:

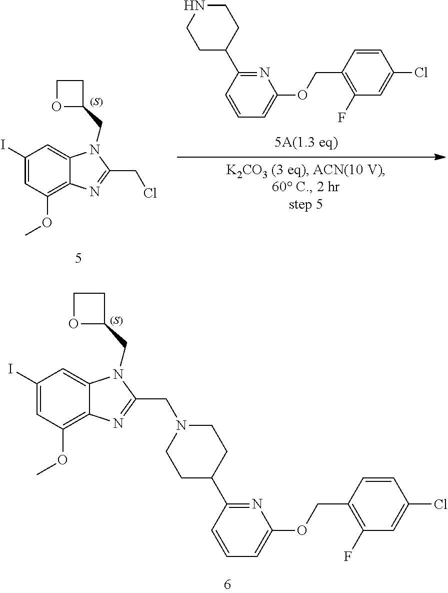

To a solution of 5 (1.1 g, 2.80 mmol, 1 eq) and 5A (1.17 g, 3.64 mmol, 1.3 eq) in ACN (11 mL) was added K₂CO₃ (1.16 g, 8.41 mmol, 3 eq). The mixture was stirred at 60° C. for 2 hr. LC-MS showed 5 was consumed completely and desired mass was detected. The mixture was filtered and concentrated under vacuum to give residue. The residue was purified by column chromatography (SiO₂, Petroleum ether/Ethyl acetate=1/0 to 0/1). 6 (1.5 g, 2.22 mmol, 79.09% yield) was obtained as a yellow solid.

LCMS: RT=0.522 min, MS cal.: 676.1, [M+H]⁺=677.1

### General Procedure for Preparation of Intermediate 8:

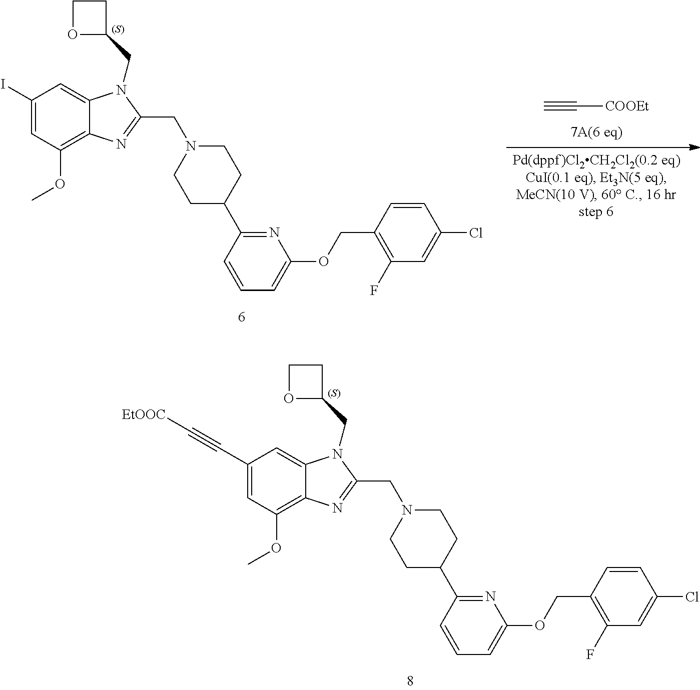

To a solution of 6 (100 mg, 147.72 μmol, 1 eq), 7A (86.95 mg, 886.33 μmol, 86.95 μL, 6 eq) and TEA (74.74 mg, 738.61 μmol, 102.81 μL, 5 eq) in ACN (1 mL) was added CuI (2.81 mg, 14.77 μmol, 0.1 eq) and Pd(dppf)Cl₂CH₂Cl₂ (24.13 mg, 29.54 μmol, 0.2 eq) under N₂ atmosphere. The mixture was degassed and purged with N₂ for 3 times. The mixture was stirred under N₂ atmosphere at 60° C. for 16 hr. LC-MS showed 6 was consumed completely and desired mass was detected. The reaction mixture was poured into H₂O (15 mL) and extracted with ethyl acetate (10 mL*3). The combined organic layers were washed with brine (10 mL), dried over Na₂SO₄, filtered, and concentrated under reduced pressure to give a residue. The residue was purified by column chromatography (SiO₂, Petroleum ether/Ethyl acetate=1/0 to 0/1). 7 (320 mg, 494.49 μmol, 27.90% yield) was obtained as a brown solid.

LCMS: RT=0.523 min, MS cal.: 646.2, [M+H]⁺=647.2

### General Procedure for Preparation of Compound 382 (9):

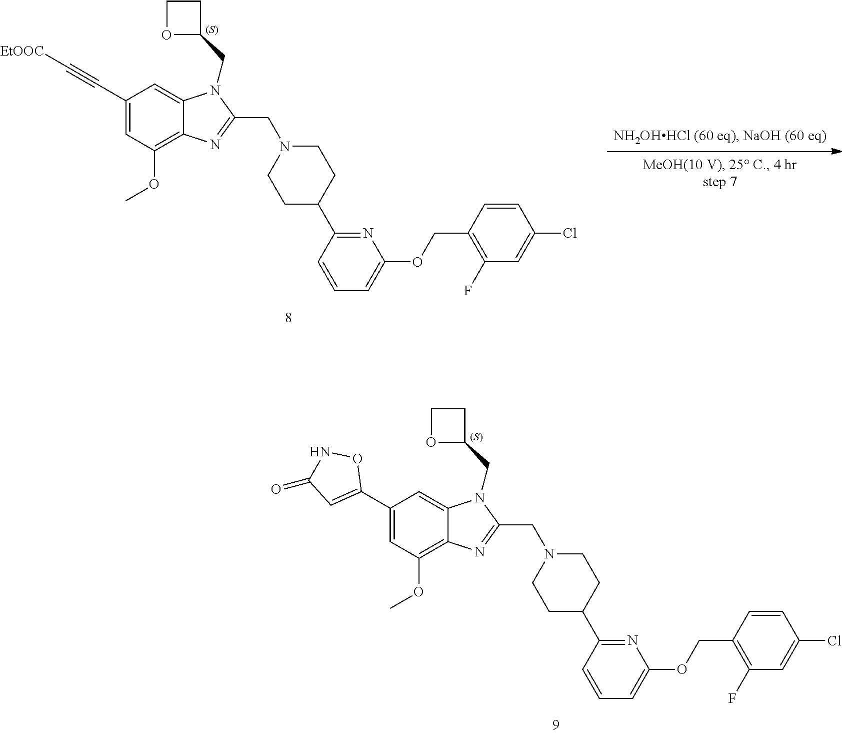

To a solution of 8 (20 mg, 30.91 μmol, 1 eq) in MeOH (0.2 mL) was added NH₂OH·HCl (128.86 mg, 1.85 mmol, 60 eq) and NaOH (140.00 mg, 3.50 mmol, 60 eq). The mixture was stirred at 25° C. for 4 hr. LC-MS showed 8 was consumed completely and desired mass was detected. The residue was poured into H₂O (20 mL) and extracted with ethyl acetate (15 mL*3). The combined organic layers were washed with brine (15 mL), dried over Na₂SO₄, filtered, and concentrated under reduced pressure to give a residue. The residue was purified by prep-HPLC (neutral condition; column: WePure Biotech XP tC18 150*40*7 um; mobile phase: [H₂O (10 mM NH₄HCO₃)-ACN]; gradient: 35%-65% B over 8.0 min). (S)-5-(2-((4-(6-((4-Chloro-2-fluorobenzyl)oxy)pyridin-2-yl)piperidin-1-yl)methyl)-4-methoxy-1-(oxetan-2-ylmethyl)-1H-benzo[d]imidazol-6-yl) isoxazol-3 (2H)-one (8.67 mg, 13.67 μmol, 7.37% yield) was obtained as a white solid.

LCMS: RT=2.953 min, MS cal.: 633.2, [M+H]⁺=634.3

HPLC: RT=11.291 min, purity: 99.31%

¹H NMR (400 MHz, METHANOL-d4) δ=7.67 (s, 1H), 7.57 (br t, J=7.6 Hz, 1H), 7.49 (br t, J=8.0 Hz, 1H), 7.28-7.08 (m, 3H), 6.82 (br d, J=7.2 Hz, 1H), 6.63 (d, J=8.0 Hz, 1H), 6.34 (br s, 1H), 5.41 (s, 2H), 5.31-5.23 (m, 1H), 4.90-4.88 (m, 1H), 4.71 (br d, J=13.6 Hz, 1H), 4.66-4.60 (m, 1H), 4.51-4.44 (m, 1H), 4.05 (s, 3H), 4.00 (br d, J=14.0 Hz, 1H), 3.93-3.86 (m, 1H), 3.05 (br d, J=10.4 Hz, 1H), 2.93 (br d, J=10.4 Hz, 1H), 2.87-2.75 (m, 1H), 2.70-2.49 (m, 2H), 2.41-2.19 (m, 2H), 1.94-1.77 (m, 4H)

### Example A259—Preparation of Compound 383. 2-((4-(6-((4-Chloro-2-fluorobenzyl)oxy)pyridin-2-yl) piperazin-1-yl)methyl)-4-methoxy-1-methyl-1H-benzo[d]imidazole-6-carboxylic acid (Compound 383) (5)

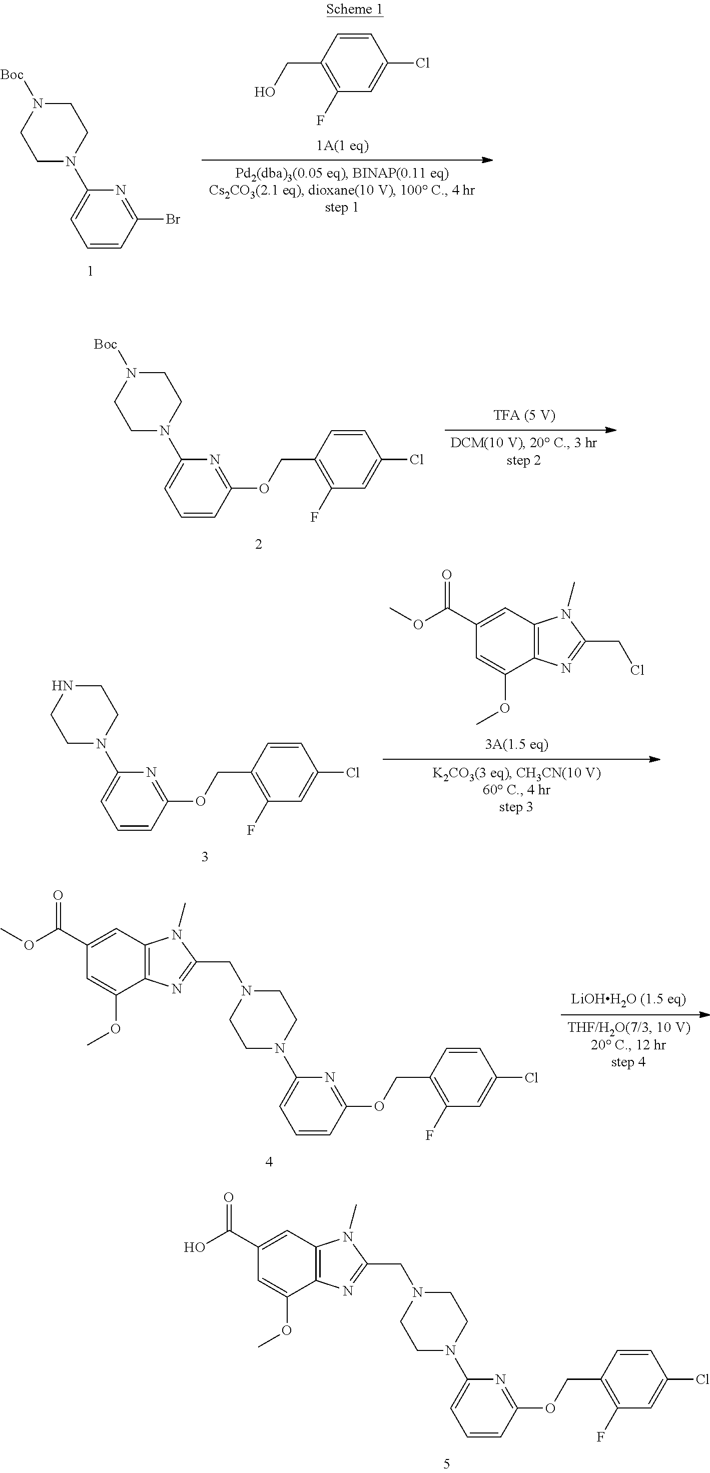

### General Procedure for Preparation of Intermediate 2:

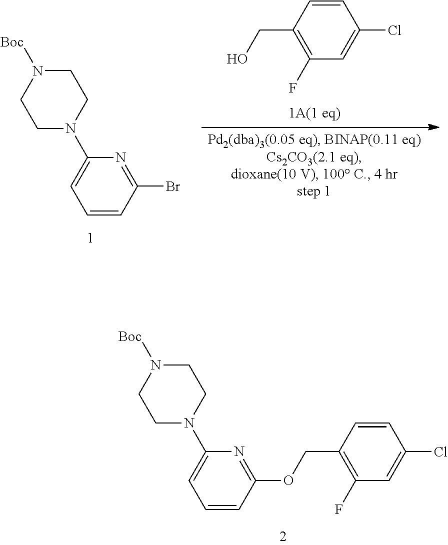

A mixture of 1 (0.9 g, 2.63 mmol, 1 eq) and 1A (422.28 mg, 2.63 mmol, 1 eq) in dioxane (9 mL) was added BINAP (180.13 mg, 289.28 μmol, 0.11 eq), Cs₂CO₃ (1.80 g, 5.52 mmol, 2.1 eq) and Pd₂(dba)₃ (120.41 mg, 131.49 μmol, 0.05 eq). The mixture was stirred at 100° C. for 4 hr under N₂ atmosphere. LCMS showed the starting material was consumed completely. The reaction mixture was diluted with H₂O (10 mL) and extracted with EA (20 mL*3). The combined organic layers were concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 62%-92% B over 8.0 min). 2 (0.33 g, 782.19 μmol, 53.54% yield) was obtained as a white solid.

LCMS: RT=1.690 min, MS cal.: 422.1, [M+H]⁺=422.16

¹H NMR (400 MHz, CHLOROFORM-d) δ=7.44 (td, J=8.0, 12.8 Hz, 2H), 7.17-7.07 (m, 2H), 6.30-6.17 (m, 2H), 5.36 (s, 2H), 3.60-3.45 (m, 8H), 1.50 (s, 9H)

### General Procedure for Preparation of Intermediate 3:

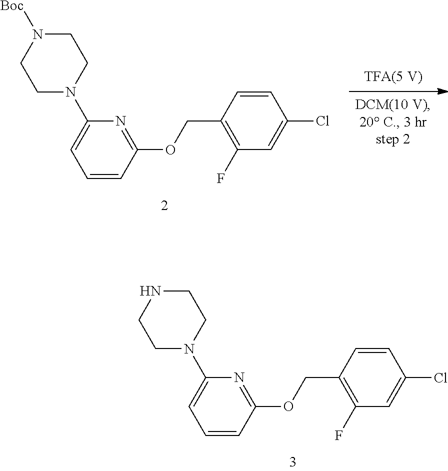 [SMILES: CC(C)(C)OC(=O)N1CCN(c2cccc(OCc3ccc(Cl)cc3F)n2)CC1 ; "DCM(10 V),20° C., 3 hrstep 2" ; "TFA(5 V)" ; Fc1cc(Cl)ccc1COc1cccc(N2CCNCC2)n1]

To a solution of 2 (0.28 g, 663.68 μmol, 1 eq) in DCM (3 mL) was added TFA (3 M, 1.26 mL, 5.68 eq). The mixture was stirred at 20° C. for 3 hr. LCMS (ET87377-94-P1A1) showed the starting material was consumed completely. The reaction mixture was concentrated under reduced pressure to give a residue. 3 (0.28 g, 642.50 μmol, 96.81% yield, TFA) was obtained as a white solid.

LCMS: RT=0.562 min, MS cal.: 322.2, [M+H]⁺=322.10

¹H NMR (400 MHz, CHLOROFORM-d) δ=7.63 (br t, J=8.0 Hz, 1H), 7.41 (br t, J=8.0 Hz, 1H), 7.22-7.07 (m, 2H), 6.39-6.31 (m, 2H), 5.38-5.27 (m, 2H), 3.88 (br s, 4H), 3.43 (br s, 4H)

### General Procedure for Preparation of Intermediate 4:

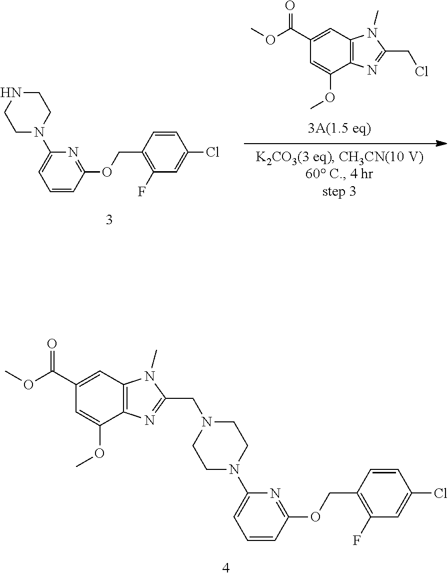

To a solution of 3 (0.14 g, 321.25 μmol, 1 eq, TFA) and 3A (129.48 mg, 481.87 μmol, 1.5 eq) in CH₃CN (2 mL) was added K₂CO₃ (133.19 mg, 963.75 μmol, 3 eq). The mixture was stirred at 60° C. for 4 hr. LCMS showed the starting material was consumed completely. The reaction mixture was diluted with H₂O (10 mL) and extracted with EA (30 mL*3). The combined organic layers were concentrated under reduced pressure to give a residue. The residue was purified by column chromatography (SiO₂, Petroleum ether/Ethyl acetate=4/1 to 2/1). 4 (0.14 g, 252.70 μmol, 78.66% yield) was obtained as a white solid.

LCMS: RT=0.700 min, MS cal.: 554.2, [M+H]⁺=554.19

¹H NMR (400 MHz, CHLOROFORM-d) δ=7.79 (s, 1H), 7.45-7.37 (m, 3H), 7.15-7.06 (m, 2H), 6.17 (br d, J=8.0 Hz, 2H), 5.34 (s, 2H), 4.08 (s, 3H), 3.97 (s, 6H), 3.50 (br d, J=3.2 Hz, 4H), 2.83-2.46 (m, 4H)

### General Procedure for Preparation of Compound 383 (5):

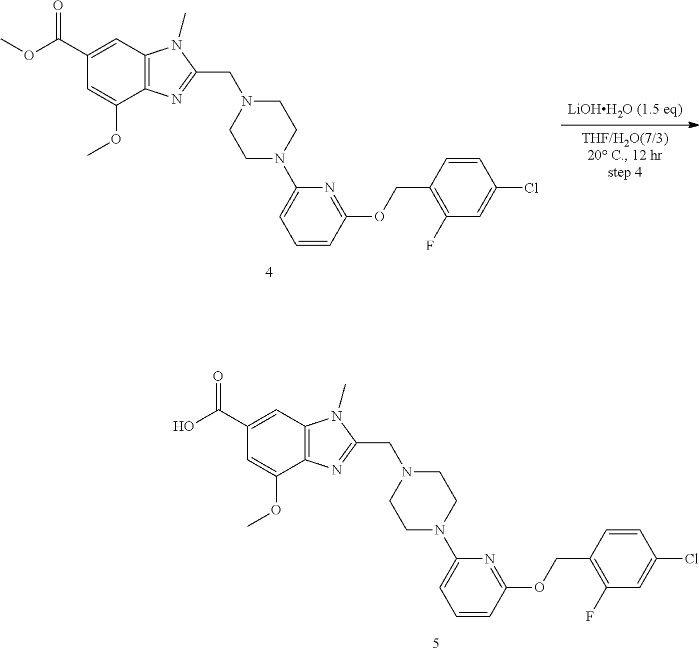 [SMILES: COC(=O)c1cc(OC)c2nc(CN3CCN(c4cccc(OCc5ccc(Cl)cc5F)n4)CC3)n(C)c2c1 ; "LiOH·H2O (1.5 eq)" ; "THF/H2O(7/3)20° C., 12 hrstep 4" ; COc1cc(C(=O)O)cc2c1nc(CN1CCN(c3cccc(OCc4ccc(Cl)cc4F)n3)CC1)n2C]

To a solution of 4 (0.14 g, 252.70 μmol, 1 eq) in THF (1.4 mL) was added LiOH H₂O (15.91 mg, 379.05 μmol, 1.5 eq) in H₂O (0.6 mL). The mixture was stirred at 20° C. for 12 hr. LCMS showed the starting material was consumed completely. The reaction mixture was diluted with THF (2 mL) The residue was purified by prep-HPLC (column: Waters Xbridge Prep OBD C18 150*40 mm*10 um; mobile phase: [H₂O (10 mM NH₄HCO₃)-ACN]; gradient: 25%-65% B over 8.0 min). 2-((4-(6-((4-Chloro-2-fluorobenzyl)oxy)pyridin-2-yl) piperazin-1-yl)methyl)-4-methoxy-1-methyl-1H-benzo[d]imidazole-6-carboxylic acid (28.6 mg, 52.96 μmol, 20.96% yield) was obtained as a white solid.

LCMS: RT=2.668 min, MS cal.: 540.2, [M+H]⁺=540.17

HPLC: RT=10.424 min, purity: 99.34%

¹H NMR (400 MHz, METHANOL-d₄) δ=7.79 (s, 1H), 7.48-7.39 (m, 3H), 7.25-7.11 (m, 2H), 6.26 (d, J=8.0 Hz, 1H), 6.09 (d, J=8.0 Hz, 1H), 5.33 (s, 2H), 4.02 (s, 3H), 3.96 (s, 3H), 3.87 (s, 2H), 3.54-3.43 (m, 4H), 2.59 (br t, J=4.8 Hz, 4H)

### Example A260—Preparation of Compound 384. 4-(((6-(1-((4-Ethoxy-1-methyl-6-(3-oxo-2,3-dihydroisoxazol-5-yl)-1H-benzo[d]imidazol-2-yl)methyl)piperidin-4-yl)pyridin-2-yl)oxy)methyl)-3-fluorobenzonitrile (Compound 384) (10)

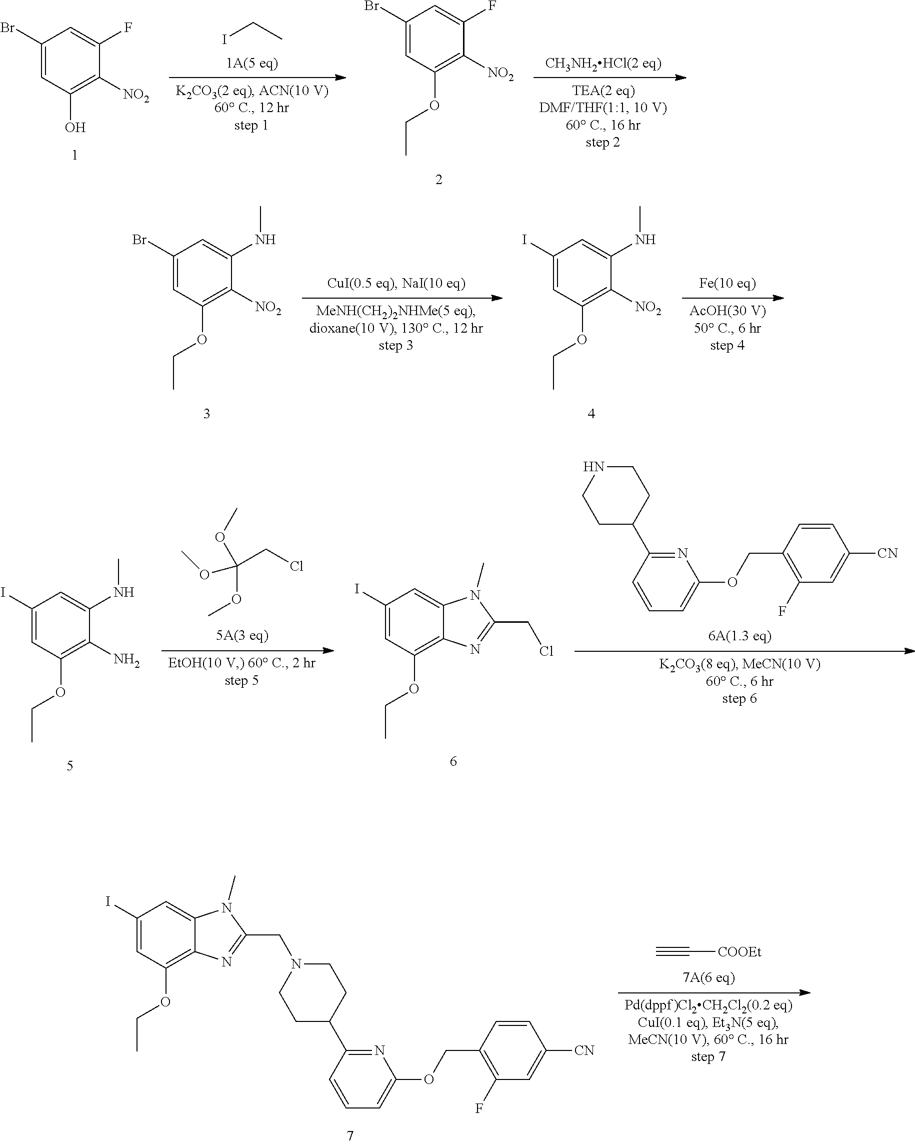

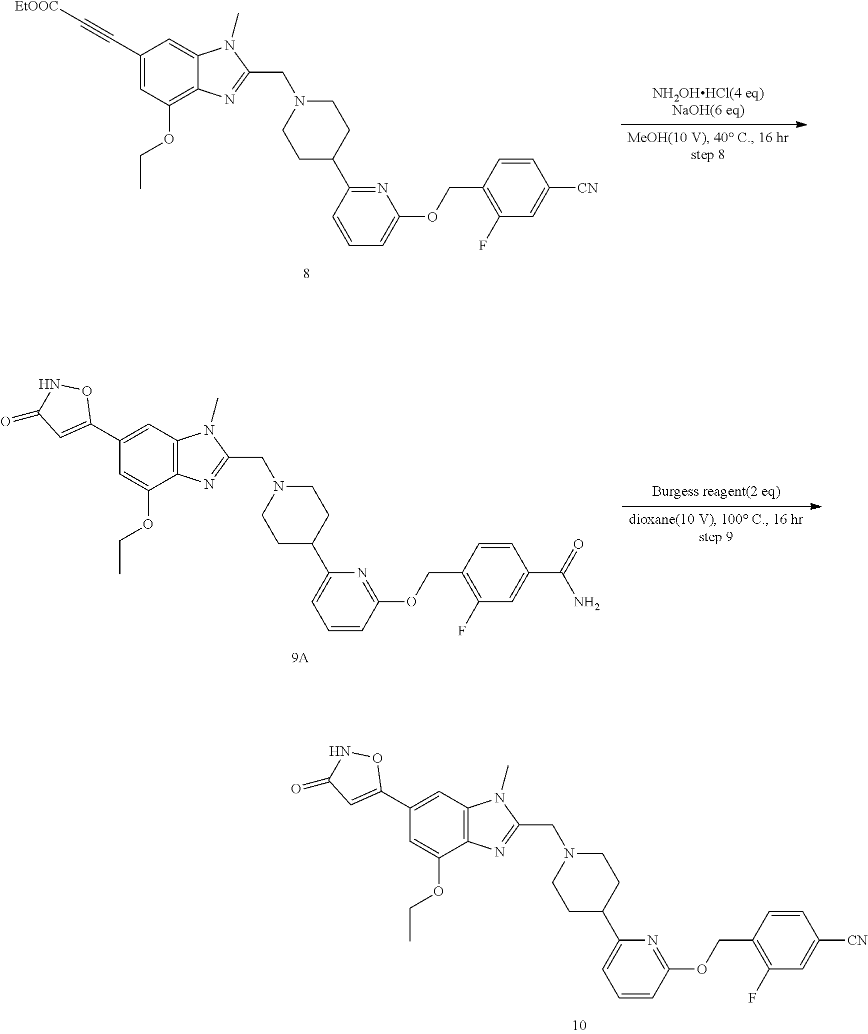

### General Procedure for Preparation of Intermediate 2:

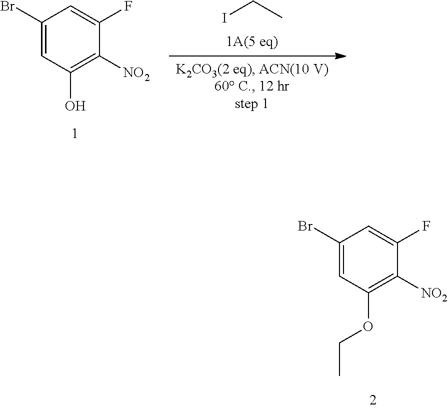

To a solution of 1 (3 g, 12.71 mmol, 1 eq) and 1A (9.91 g, 63.56 mmol, 5.08 mL, 5 eq) in MeCN (30 mL) was added K₂CO₃ (3.51 g, 25.42 mmol, 2 eq). The mixture was stirred at 60° C. for 12 hr. LC-MS showed 1 was consumed completely and desired mass was detected. The suspension was filtered, and the filtrate was concentrated under reduced pressure to give a residue. The residue was purified by column chromatography (SiO₂, Petroleum ether/Ethyl acetate=1/0 to 0/1). 2 (2.52 g, 9.54 mmol, 75.08% yield) was obtained as a yellow solid. 10

¹H NMR (400 MHz, DMSO-d6) δ=7.55 (dd, J=1.6, 9.6 Hz, 1H), 7.50 (s, 1H), 4.27 (q, J=6.8 Hz, 2H), 1.30 (t, J=6.8 Hz, 3H)

### General Procedure for Preparation of Intermediate 3:

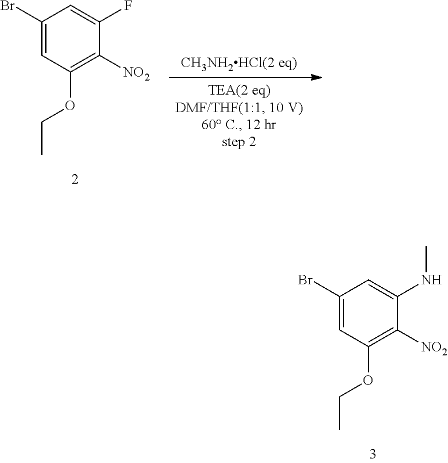

To a solution of 2 (4.2 g, 15.91 mmol, 1 eq) in DMF (21 mL) and THF (21 mL) was added CH₃NH₂·HCl (2.15 g, 31.81 mmol, 2 eq) and TEA (3.22 g, 31.81 mmol, 4.43 mL, 2 eq). The mixture was stirred at 60° C. for 12 hr. LC-MS showed 2 was consumed completely and desired mass was detected. The residue was poured into H₂O (100 mL) and extracted with ethyl acetate (100 mL*3). The combined organic phase was washed with brine (100 mL*3), dried over Na₂SO₄, filtered, and concentrated under reduced pressure to give a residue. The residue was purified by column chromatography (SiO₂, Petroleum ether/Ethyl acetate=1/0 to 0/1). 3 (3.8 g, 13.71 mmol, 86.16% yield) was obtained as a yellow solid.

¹H NMR (400 MHz, DMSO-d6) δ=6.61 (d, J=1.6 Hz, 1H), 6.55 (d, J=1.6 Hz, 1H), 6.42 (q, J=4.8 Hz, 1H), 4.10 (q, J=6.8 Hz, 2H), 2.72 (d, J=4.8 Hz, 3H), 1.25 (t, J=6.8 Hz, 3H).

### General Procedure for Preparation of Intermediate 4:

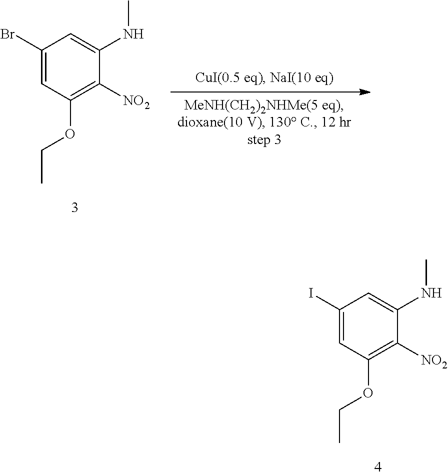 [6482] To a mixture of 3 (1 g, 3.64 mmol, 1 eq), NaI (5.45 g, 36.35 mmol, 10 eq) and MeNH(CH₂)₂NHMe (1.60 g, 18.18 mmol, 1.96 mL, 5 eq) in dioxane (10 mL) was added CuI (346.15 mg, 1.82 mmol, 0.5 eq) under N₂ atmosphere. The mixture was degassed and purged with N₂ for 3 times. The mixture was stirred under N₂ atmosphere at 130° C. for 12 hr. LC-MS showed 3 was consumed completely and desired mass was detected. The residue was poured into H₂O (20 mL) and extracted with ethyl acetate (20 mL*3). The combined organic layers were washed with brine (20 mL), dried over Na₂SO₄, filtered and concentrated under reduced pressure to give a residue. The residue was purified by column chromatography (SiO₂, petroleum ether/ethyl acetate=1/0 to 0/1). 4 (1 g, 2.70 mmol, 74.41% yield) was obtained as a yellow solid.

¹H NMR (400 MHz, DMSO-d6) δ=6.73-6.72 (m, 2H), 6.31 (q, J=1.6 Hz, 1H), 4.09 (q, J=6.8 Hz, 2H), 2.67 (s, 3H), 1.30 (t, J=6.8 Hz, 3H)

### General Procedure for Preparation of Intermediate 5:

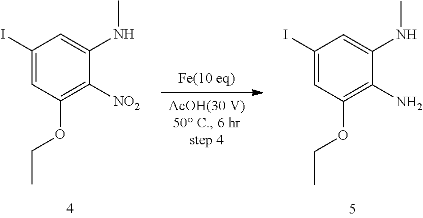

To a solution of 4 (1 g, 3.10 mmol, 1 eq) in AcOH (30 mL) was added Fe (1.73 g, 31.05 mmol, 10 eq). The mixture was stirred at 50° C. for 6 hr. LC-MS showed 4 was consumed completely and desired mass was detected. The reaction mixture was filtered through a Celite pad and washed by ethyl acetate (10 mL*3). The filtrate was concentrated under reduced pressure to give the residue. The residue was purified by column chromatography (SiO₂, petroleum ether/ethyl acetate=1/0 to 1/1). 5 (770 mg, 2.16 mmol, 69.62% yield) was obtained as a yellow solid.

¹H NMR (400 MHz, DMSO-d6) δ=6.55 (d, J=1.6 Hz, 1H), 6.36 (d, J=1.6 Hz, 1H), 4.87 (br s, 1H), 4.14 (br s, 2H), 3.94 (q, J=6.8 Hz, 2H), 2.67 (s, 3H), 1.30 (t, J=6.8 Hz, 3H)

### General Procedure for Preparation of Intermediate 6:

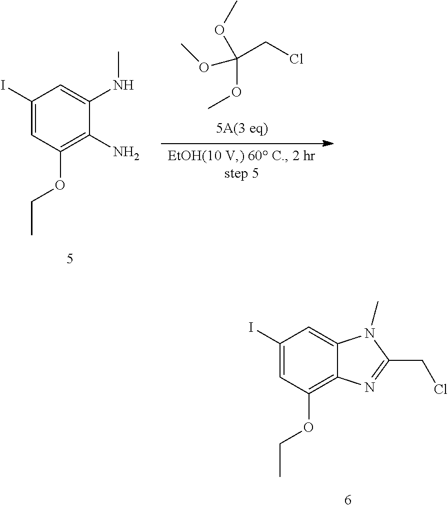

To a solution of 5 (770 mg, 2.64 mmol, 1 eq) in EtOH (7.7 mL) was added 5A (1.22 g, 7.91 mmol, 3 eq). The mixture was stirred at 60° C. for 2 hr. LC-MS showed 5 was consumed completely and desired mass was detected. The reaction mixture was concentrated under reduced pressure to give a residue. The residue was purified by column chromatography (SiO₂, petroleum ether/ethyl acetate=8/1 to 1/1). 6 (708 mg, 2.02 mmol, 76.61% yield) was obtained as a yellow solid.

¹H NMR (400 MHz, DMSO-d6) δ=7.59 (d, J=1.2 Hz, 1H), 6.98 (d, J=1.2 Hz, 1H), 5.04 (s, 2H), 4.22 (q, J=6.8 Hz, 2H), 3.80 (s, 3H), 1.37 (t, J=6.8 Hz, 3H)

### General Procedure for Preparation of Intermediate 7:

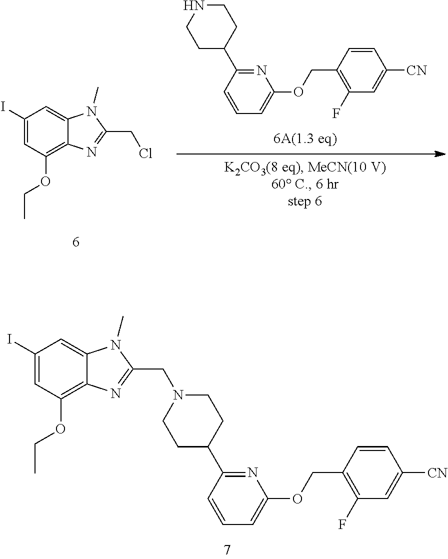 [SMILES: CCOc1cc(I)cc2c1nc(CCl)n2C ; N#Cc1ccc(COc2cccc(C3CCNCC3)n2)c(F)c1 ; "6A(1.3 eq)" ; "K2CO3(8 eq), MeCN(10 V)60° C., 6 hrstep 6" ; CCOc1cc(I)cc2c1nc(CN1CCC(c3cccc(OCc4ccc(C#N)cc4F)n3)CC1)n2C]

To a mixture of 6 (400 mg, 1.14 mmol, 1 eq) and 6A (461.81 mg, 1.48 mmol, 1.3 eq) in MeCN (4 mL) was added K₂CO₃ (1.26 g, 9.13 mmol, 8 eq). The mixture was stirred at 60° C. for 6 hr under N₂ atmosphere. LC-MS showed 6 was consumed completely and desired mass was detected. The mixture was filtered, and the filtrate was concentrated under reduced pressure to give a residue. The residue was purified by column chromatography (SiO₂, Petroleum ether/Ethyl acetate=2/1 to 0/1). 7 (620 mg, 991.25 μmol, 86.88% yield) was obtained as a yellow solid.

¹H NMR (400 MHz, DMSO-d6) δ=7.88 (d, J=10.0 Hz, 1H), 7.69 (d, J=3.6 Hz, 2H), 7.64 (t, J=8.0 Hz, 1H), 7.54 (d, J=1.2 Hz, 1H), 6.93 (d, J=1.2 Hz, 1H), 6.87 (d, J=7.2 Hz, 1H), 6.71 (d, J=8.0 Hz, 1H), 5.45 (s, 2H), 4.21 (q, J=6.8 Hz, 2H), 3.81 (s, 3H), 3.74 (s, 2H), 2.87 (d, J=11.2 Hz, 2H), 2.60-2.56 (m, 1H), 2.15 (t, J=10.0 Hz, 2H), 1.76-1.61 (m, 4H), 1.38 (t, J=6.8 Hz, 3H)

### General Procedure for Preparation of Intermediate 8:

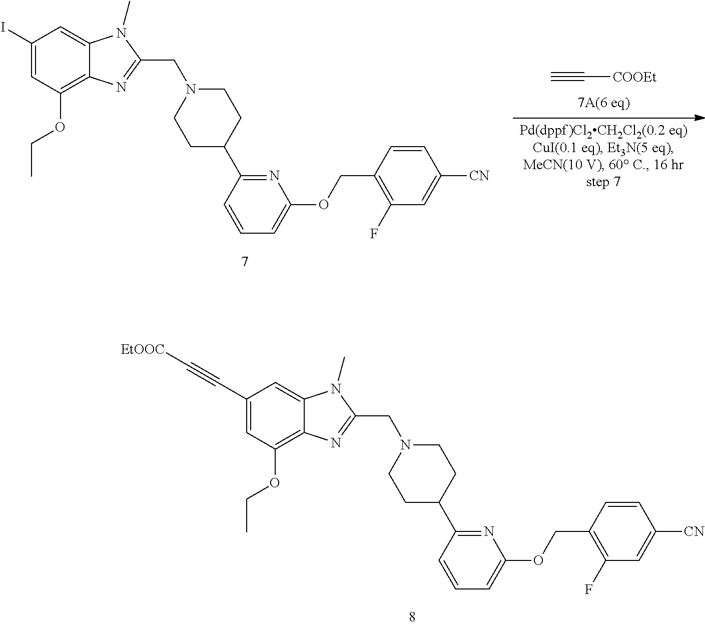

To a mixture of 7 (100 mg, 159.88 μmol, 1 eq), CuI (3.04 mg, 15.99 μmol, 0.1 eq) and Et₃N (80.89 mg, 799.39 μmol, 111.27 μL, 5 eq) in MeCN (1 mL) was added Pd(dppf)Cl₂·CH₂Cl₂ (26.11 mg, 31.98 μmol, 0.2 eq) and 7A (94.10 mg, 959.27 μmol, 94.10 μL, 6 eq) under N₂ atmosphere, and the mixture was degassed and purged with N₂ for 3 times, and stirred under N₂ atmosphere at 60° C. for 6 hr. And the reaction was set 6 batches in parallel. LC-MS showed 7 was consumed completely and desired mass was detected. The mixture was combined, diluted with H₂O (10 mL) and extracted with EtOAc (10 mL*3). The combined organic layers were washed with brine (10 mL), dried over Na₂SO₄, filtered, and concentrated under reduced pressure to give a residue. The residue was purified by prep-HPLC (basic condition; column: WePure Biotech XP tC18 150*40*7 um; mobile phase: [H₂O (0.05% NH₃H₂O+10 mM NH₄HCO₃)-ACN]; gradient: 60%-90% B over 8.0 min). 8 (230 mg, 386.13 μmol, 40.25% yield) was obtained as a brown solid.

¹H NMR (400 MHz, DMSO-d6) δ=7.88 (d, J=10.0 Hz, 1H), 7.69-7.61 (m, 4H), 6.90-6.86 (m, 2H), 6.71 (d, J=8.0 Hz, 1H), 5.45 (s, 2H), 4.24 (q, J=6.8 Hz, 4H), 3.88 (s, 3H), 3.79 (s, 2H), 2.89 (d, J=11.2 Hz, 2H), 2.34-2.31 (m, 1H), 2.17 (t, J=10.0 Hz, 2H), 1.76-1.61 (m, 4H), 1.40 (t, J=6.8 Hz, 3H), 1.27 (t, J=6.8 Hz, 3H)

### General Procedure for Preparation of Intermediate 9A:

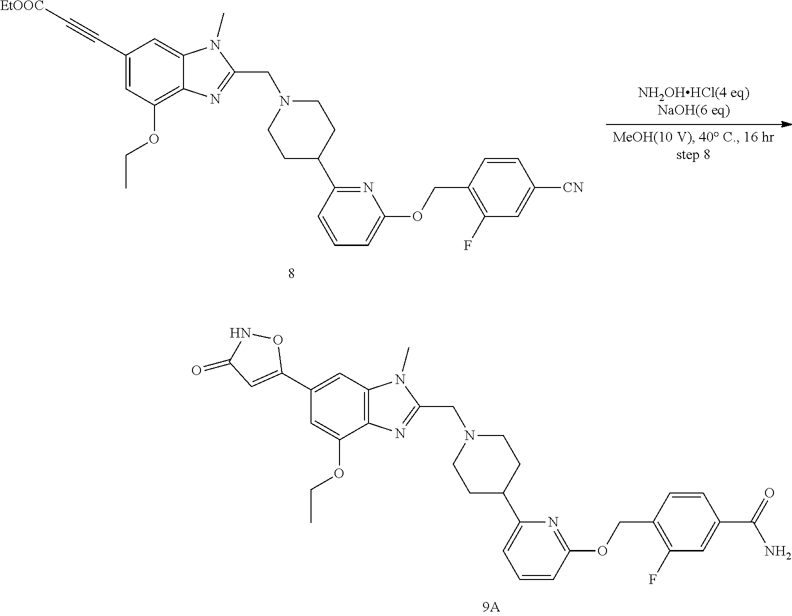 [SMILES: CCOC(=O)C#Cc1cc(OCC)c2nc(CN3CCC(c4cccc(OCc5ccc(C#N)cc5F)n4)CC3)n(C)c2c1 ; "MeOH(10 V), 40° C., 16 hrstep 8" ; "NH2OH·HCl(4 eq)NaOH(6 eq)" ; CCOc1cc(-c2cc(=O)[nH]o2)cc2c1nc(CN1CCC(c3cccc(OCc4ccc(C(N)=O)cc4F)n3)CC1)n2C]

To a mixture of 8 (120 mg, 201.46 μmol, 1 eq) and NH₂OH·HCl (56.00 mg, 805.83 μmol, 4 eq) in MeOH (1.2 mL) was added NaOH (48.35 mg, 1.21 mmol, 6 eq). The mixture was stirred at 40° C. for 16 hr. LC-MS showed 8 was consumed completely and desired mass was detected. The reaction mixture was concentrated under reduced pressure to give a residue. 9A (210 mg, crude) was obtained as a brown solid.

LCMS: RT=1.031 min, MS cal.: 600.2, [M+H]⁺=601.2

### General Procedure for Preparation of Compound 384 (10):

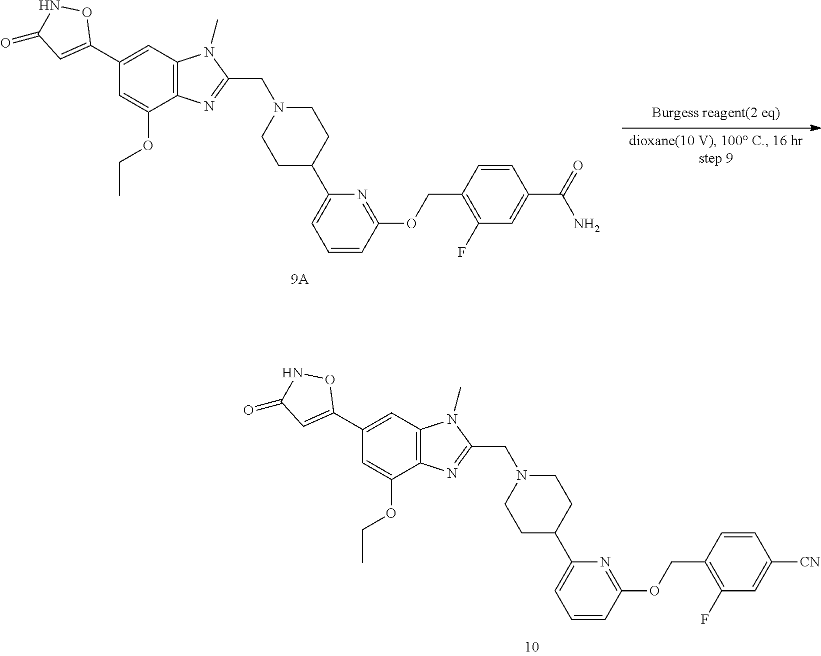 [SMILES: CCOc1cc(-c2cc(=O)[nH]o2)cc2c1nc(CN1CCC(c3cccc(OCc4ccc(C(N)=O)cc4F)n3)CC1)n2C ; "Burgess reagent(2 eq)" ; "dioxane(10 V), 100° C., 16 hrstep 9" ; CCOc1cc(-c2cc(=O)[nH]o2)cc2c1nc(CN1CCC(c3cccc(OCc4ccc(C#N)cc4F)n3)CC1)n2C]

To a solution of 9A (80 mg, 133.19 μmol, 1 eq) in dioxane (0.8 mL) was added Burgess regent (63.80 mg, 267.72 μmol, 2 eq). The mixture was stirred at 100° C. for 16 hr. LC-MS showed 9A was consumed completely and desired mass was detected. The reaction mixture was quenched by addition H₂O (2 mL) at 25° C. and extracted with EtOAc (5 mL*3). The combined organic layers were washed with brine (5 mL), dried over Na₂SO₄, filtered, and concentrated under reduced pressure to give residue. The residue was purified by prep-HPLC (basic condition; column: WePure Biotech XP tC18 150*40*7 um; mobile phase: [H₂O (0.05% NH₃H₂O+10 mM NH₄HCO₃)-ACN]; gradient: 35%-65% B over 8.0 min). 4-(((6-(1-((4-Ethoxy-1-methyl-6-(3-oxo-2,3-dihydroisoxazol-5-yl)-1H-benzo[d]imidazol-2-yl)methyl)piperidin-4-yl)pyridin-2-yl)oxy)methyl)-3-fluorobenzonitrile (11.98 mg, 20.26 μmol, 15.21% yield, 98.55% purity) was obtained as a brown solid.

LCMS: RT=2.228 min, MS cal.: 582.2, [M+H]⁺=583.2

HPLC: RT=10.695 min, purity: 98.55%

¹H NMR (400 MHz, METHANOL-d4) δ=7.66 (t, J=7.6 Hz, 1H), 7.60-7.51 (m, 4H), 7.13 (s, 1H), 6.83 (d, J=7.2 Hz, 1H), 6.67 (d, J=8.0 Hz, 1H), 6.37 (s, 1H), 5.50 (s, 2H), 4.31 (q, J=7.2 Hz, 2H), 3.98 (s, 3H), 3.87 (s, 2H), 3.00 (d, J=11.2 Hz, 2H), 2.64-2.59 (m, 1H), 2.33-2.26 (m, 2H), 1.84-1.77 (m, 4H), 1.54 (t, J=7.2 Hz, 3H).

### Example A261—Preparation of Compound 385. 4-(Difluoromethoxy)-1-methyl-2-((4-(6-((4-(trifluoromethyl)benzyl)oxy)pyridin-2-yl)piperidin-1-yl)methyl)-1H-benzo[d]imidazole-6-carboxylic acid (Compound 385) (3)

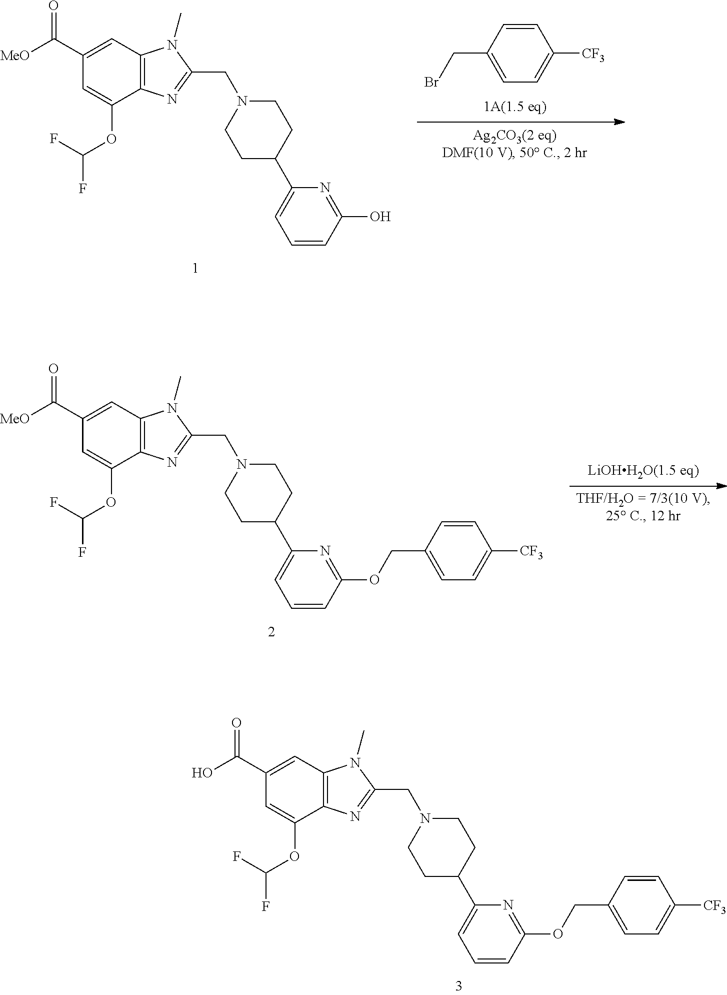

### General Procedure for Preparation of Intermediate 2:

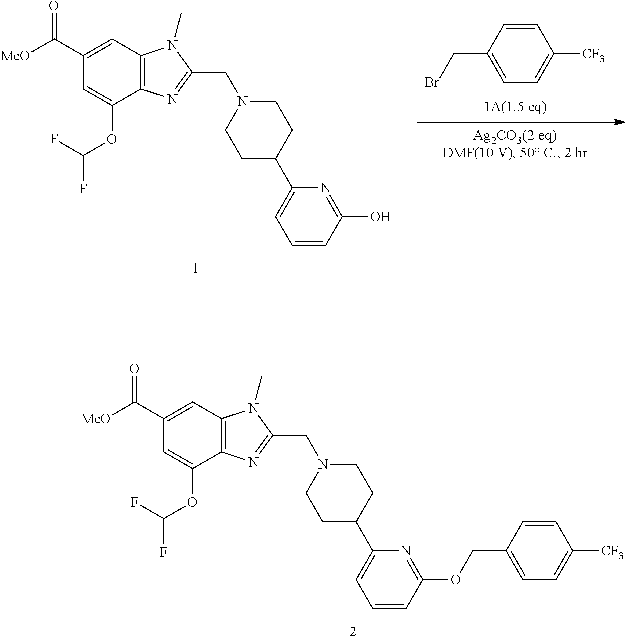

A mixture of 1 (100 mg, 223.99 μmol, 1 eq), 1A (80.31 mg, 335.99 μmol, 51.95 μL, 1.5 eq), Ag₂CO₃ (123.53 mg, 447.98 μmol, 20.33 μL, 2 eq) in DMF (1 mL) was degassed and purged with N₂ for 3 times, and then the mixture was stirred at 50° C. for 2 hr under N₂ atmosphere. LCMS showed 1 was consumed completely and one main peak with desired mass was detected. The reaction mixture was filtered and diluted with H₂O (10 mL) and extracted with EtOAc (10 mL*3). The combined organic layers were washed with brine (10 mL*3), dried over Na₂SO₄, filtered and concentrated under reduced pressure to give a residue. The residue was purified by column chromatography (SiO₂, Petroleum ether/Ethyl acetate=10/1 to 0/1). 2 (80 mg, 132.33 μmol, 59.08% yield) was obtained as a yellow solid.

LCMS: RT=0.512 min, MS cal.: 604.5, [M+H]⁺=605.4

¹H NMR (400 MHz, CHLOROFORM-d) δ=7.99 (s, 1H), 7.72 (s, 1H), 7.64-7.48 (m, 5H), 7.31 (s, 1H), 6.75 (d, J=7.2 Hz, 1H), 6.64 (d, J=8.4 Hz, 1H), 5.44 (s, 2H), 4.00 (br s, 2H), 3.97 (s, 6H), 3.94-3.84 (m, 2H), 2.94-2.86 (m, 2H), 2.69-2.57 (m, 1H), 2.42-2.19 (m, 2H), 1.97-1.70 (m, 4H)

### General Procedure for Preparation of Compound 385 (3):

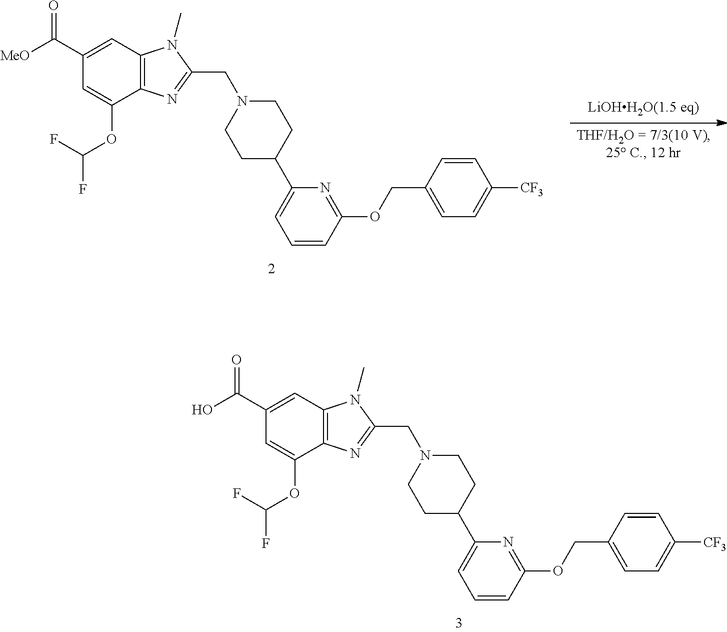

To a solution of 2 (70 mg, 115.79 μmol, 1 eq) in THF (0.7 mL) and H₂O (0.3 mL) was added LiOH·H₂O (7.29 mg, 173.68 μmol, 1.5 eq). The mixture was stirred at 25° C. for 12 hr. LCMS showed 2 was consumed completely and one main peak with desired mass was detected. The reaction mixture was filtered and concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 15%-65% B over 8.0 min). 4-(Difluoromethoxy)-1-methyl-2-((4-(6-((4-(trifluoromethyl)benzyl)oxy)pyridin-2-yl)piperidin-1-yl)methyl)-1H-benzo[d]imidazole-6-carboxylic acid (24.71 mg, 41.74 μmol, 36.05% yield, 99% purity) was obtained as a white solid.

LCMS: RT=2.893 min, MS cal.: 590.2, [M+H]⁺=591.2

HPLC: RT=12.599 min, purity: 99.75%

¹H NMR (400 MHz, DMSO-d6) δ=8.07 (s, 1H), 7.74-7.68 (m, 2H), 7.67-7.60 (m, 3H), 7.62 (t, J=74.4 Hz, 1H), 6.86 (d, J=7.2 Hz, 1H), 6.71 (d, J=8.4 Hz, 1H), 5.43 (s, 2H), 3.96 (s, 3H), 3.84 (s, 2H), 2.92 (br d, J=11.2 Hz, 2H), 2.57 (br d, J=3.6 Hz, 1H), 2.20 (br t, J=10.8 Hz, 2H), 1.80-1.61 (m, 4H)

### Example A262—Preparation of Compound 220. 2-((4-(6-((4-Chloro-2-fluorobenzyl)oxy)pyridin-2-yl)piperidin-1-yl)methyl)-4-(difluoromethoxy)-1-methyl-1H-benzo[d]imidazole-5-carboxylic acid (Compound 220) (8)

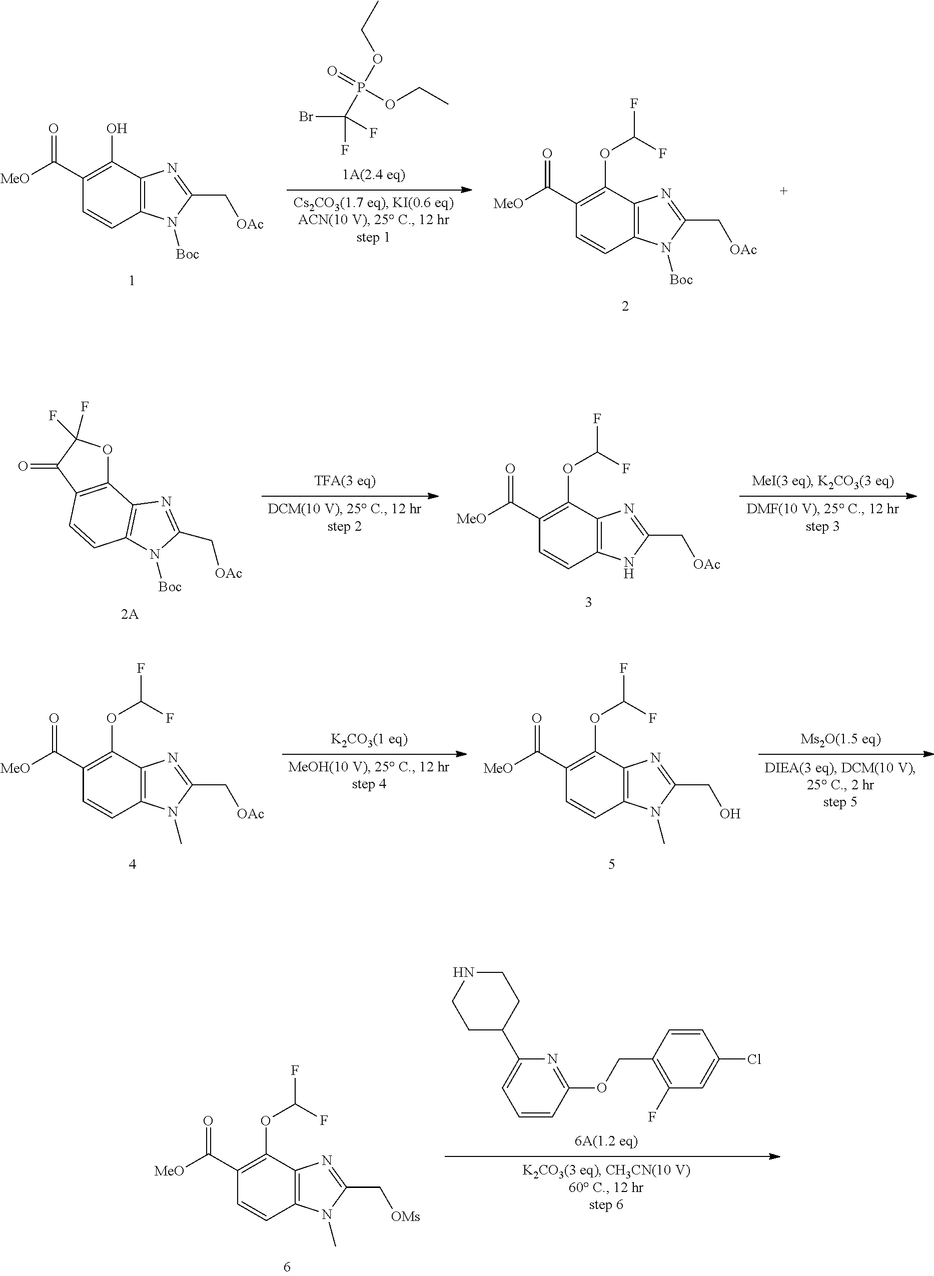

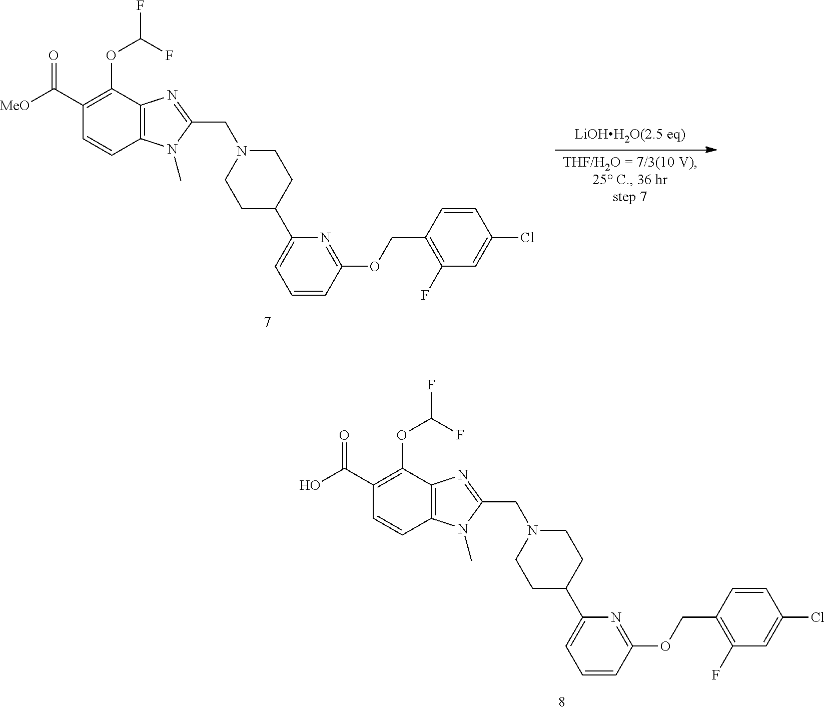

### General Procedure for Preparation of Intermediate 2:

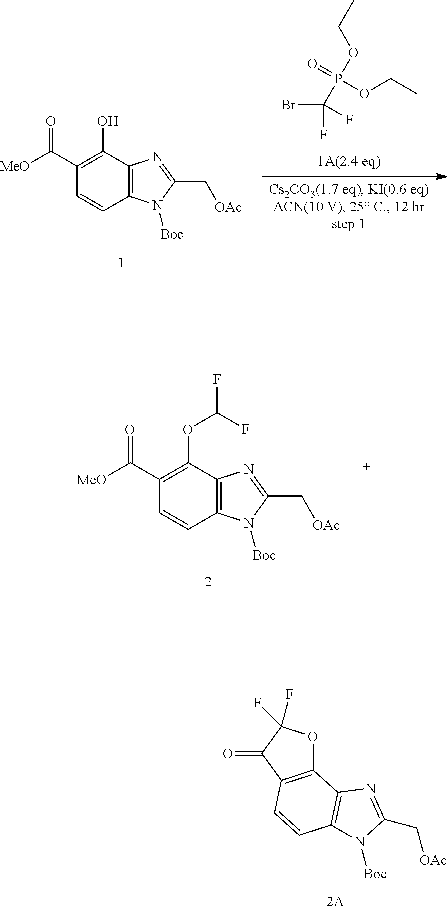

The reactions was set up in 18 batches. A mixture of 1 (200 mg, 548.92 μmol, 1 eq), 1A (351.76 mg, 1.32 mmol, 2.4 eq), Cs₂CO₃ (304.05 mg, 933.17 μmol, 1.7 eq) and KI (54.67 mg, 329.35 μmol, 0.6 eq) in ACN (10 mL) was degassed and purged with N₂ for 3 times, and then the mixture was stirred at 25° C. for 12 hr under N₂ atmosphere. TLC (Petroleum ether/Ethyl acetate=2/1, R_{f}=0.43) indicated 30% of 1 remained, and one major new spot with lower polarity was detected. 18 reactions were combined for workup. The reaction mixture was diluted with H₂O 20 mL and extracted with DCM (20 mL*4). The combined organic layers dried over Na₂SO₄, filtered, and concentrated under reduced pressure to give a residue. The combined organic layers were dried over Na₂SO₄, filtered and concentrated under reduced pressure to give a residue. The residue was purified by column chromatography (SiO₂, Petroleum ether/Ethyl acetate=100/1 to 0/1). A mixture of 2 & 2A (2.8 g crude, ratio: 1/3 in HNMR) was obtained as a yellow solid.

### General Procedure for Preparation of Intermediate 3:

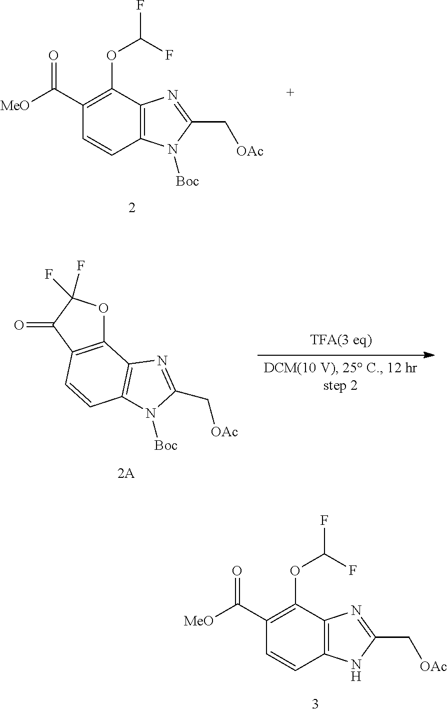 [SMILES: COC(=O)c1ccc2c(nc(COC(C)=O)n2C(=O)OC(C)(C)C)c1OC(F)F ; CC(=O)OCc1nc2c3c(ccc2n1C(=O)OC(C)(C)C)C(=O)C(F)(F)O3 ; "DCM(10 V), 25° C., 12 hrstep 2" ; "TFA(3 eq)" ; COC(=O)c1ccc2[nH]c(COC(C)=O)nc2c1OC(F)F]

A mixture of 2 (1 g crude, ratio: 1/3 in HNMR), TFA (825.56 mg, 1.81 mmol, 134.45 μL, 3 eq) in DCM (10 mL) was degassed and purged with N₂ for 3 times, and then the mixture was stirred at 25° C. for 12 hr under N₂ atmosphere. LC-MS (RT=0.933 min) showed 2 was consumed completely and one main peak with desired mass was detected. The reaction mixture was diluted with H₂O 10 mL and extracted with DCM (10 mL*3). The combined organic layers were dried over Na₂SO₄, filtered, and concentrated under reduced pressure to give a residue. The residue was purified by column chromatography (SiO₂, Petroleum ether/Ethyl acetate=100/1 to 0/1). 3 (130 mg, 413.70 μmol, 38% yield) was obtained as a white solid.

LCMS: RT=0.924 min, MS cal.: 314.2, [M+H]⁺=314.9

¹H NMR (400 MHz, DMSO-d₆) δ=7.71 (d, J=8.5 Hz, 1H), 7.50 (dd, J=2.4, 8.4 Hz, 1H), 5.30 (s, 2H), 3.84 (s, 3H), 2.13 (s, 3H)

### General Procedure for Preparation of Intermediate 4:

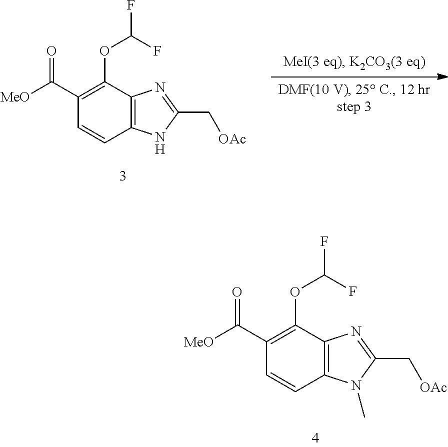 [SMILES: COC(=O)c1ccc2[nH]c(COC(C)=O)nc2c1OC(F)F ; "DMF(10 V), 25° C., 12 hrstep 3" ; "MeI(3 eq), K2CO3(3 eq)" ; COC(=O)c1ccc2c(nc(COC(C)=O)n2C)c1OC(F)F]

DMF (0.5 mL) was charged to the thumb bottle, then 3 (120 mg, 381.87 μmol, 1 eq) and K₂CO₃ (158.33 mg, 1.15 mmol, 3 eq) was added to the mixture at 25° C. At 25° C. (inner temperature), CH₃I (162.61 mg, 1.15 mmol, 71.32 μL, 3 eq) was added to the reaction mixture at 25° C. After the addition, the mixture was stirred at 25° C. for 12 hr. LCMS (RT=1.048 min) showed 3 was consumed completely and one main peak with desired mass was detected. The reaction mixture was added to H₂O 10 mL at 25° C. The mixture was extracted by DCM (10 mL*4). Then organic phase was combined and washed by brine (10 mL), dried with Na₂SO₄, filtered. The organic phase was concentrated under reduced pressure at 35° C. to give a residue. The residue was purified by column chromatography (SiO₂, Petroleum ether/Ethyl acetate=100/1 to Ethyl acetate/Methanol=0/1). 4 (100 mg, 304.63 μmol, 79% yield) was obtained as a yellow oil.

LCMS: RT=1.027 min, MS cal.: 328.3, [M+H]⁺=328.9

¹H NMR (400 MHz, DMSO-d₆) δ=7.77 (d, J=8.4 Hz, 1H), 7.67 (t, J=74.8 Hz, 1H), 7.61 (d, J=8.8 Hz, 1H), 5.37 (s, 2H), 3.87 (s, 3H), 3.85 (s, 3H), 2.12-2.10 (m, 3H)

### General Procedure for Preparation of Intermediate 5:

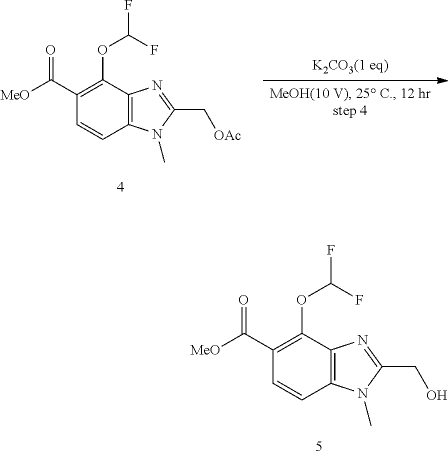 [SMILES: COC(=O)c1ccc2c(nc(COC(C)=O)n2C)c1OC(F)F ; "K2CO3(1 eq)" ; "MeOH(10 V), 25° C., 12 hrstep 4" ; COC(=O)c1ccc2c(nc(CO)n2C)c1OC(F)F]

A mixture of 4 (90 mg, 274.17 μmol, 1 eq), K₂CO₃ (37.89 mg, 274.17 μmol, 1 eq) in MeOH (0.9 mL) was degassed and purged with N₂ for 3 times, and then the mixture was stirred at 25° C. for 12 hr under N₂ atmosphere. LCMS (RT=0.872 min) showed 4 was consumed completely and one main peak with desired mass was detected. The reaction mixture was filtered and concentrated under reduced pressure to give a residue. 5 (85 mg, crude) was obtained as a yellow solid.

### General Procedure for Preparation of Intermediate 6:

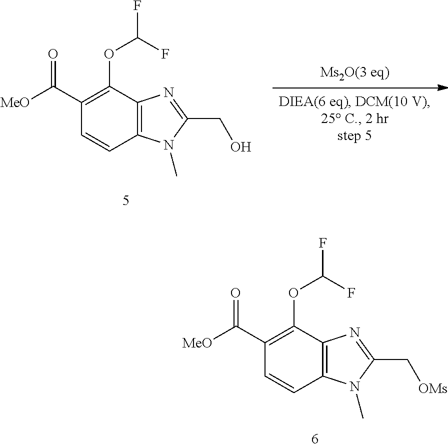 [SMILES: COC(=O)c1ccc2c(nc(CO)n2C)c1OC(F)F ; "DIEA(6 eq), DCM(10 V),25° C., 2 hrstep 5" ; "Ms2O(3 eq)" ; COC(=O)c1ccc2c(nc(COS(C)(=O)=O)n2C)c1OC(F)F]

A mixture of 5 (75 mg, 262.03 μmol, 1 eq), Ms₂O (136.93 mg, 786.08 μmol, 3 eq), DIEA (203.19 mg, 1.57 mmol, 273.84 μL, 6 eq) in DCM (0.7 mL) was degassed and purged with N₂ for 3 times, and then the mixture was stirred at 25° C. for 2 hr under N₂ atmosphere. TLC (Petroleum ether/Ethyl acetate=1/1, R_{f}=0.43) indicated 5 was consumed, and one major new spot with lower polarity was detected. The reaction mixture was added HCOOH at 25° C. until pH=7-8, and then diluted with H₂O 7 mL and extracted with DCM 7 mL*4. The combined organic layers were dried over Na₂SO₄, filtered, and concentrated under reduced pressure to give a residue. 6 (95 mg, crude) was obtained as a white solid.

### General Procedure for Preparation of Intermediate 7:

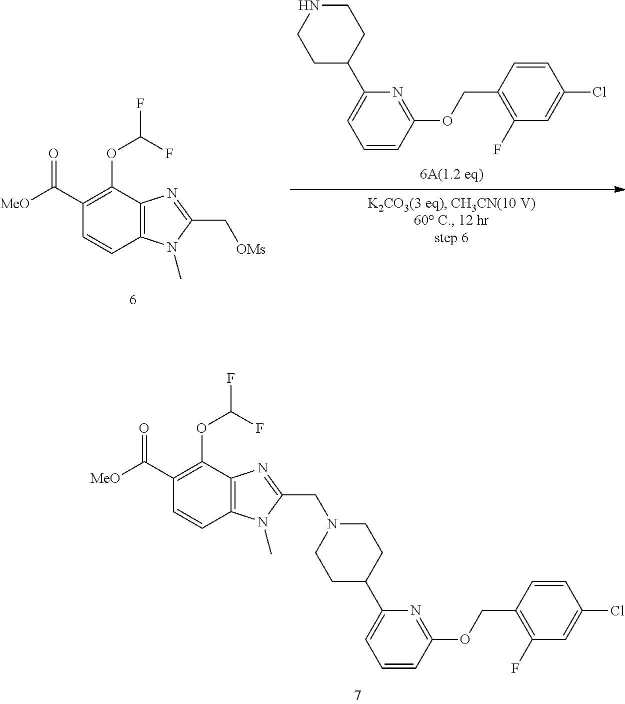 [SMILES: COC(=O)c1ccc2c(nc(COS(C)(=O)=O)n2C)c1OC(F)F ; Fc1cc(Cl)ccc1COc1cccc(C2CCNCC2)n1 ; "6A(1.2 eq)" ; "K2CO3(3 eq), CH3CN(10 V)60° C., 12 hrstep 6" ; COC(=O)c1ccc2c(nc(CN3CCC(c4cccc(OCc5ccc(Cl)cc5F)n4)CC3)n2C)c1OC(F)F]

A mixture of 6 (95 mg, 260.76 μmol, 1 eq), 6A (100.38 mg, 312.91 μmol, 1.2 eq), K₂CO₃ (108.11 mg, 782.28 μmol, 3 eq) in ACN (1 mL) was degassed and purged with N₂ for 3 times, and then the mixture was stirred at 60° C. for 12 hr under N₂ atmosphere. LCMS (RT=1.734 min) showed 6 was consumed completely and one main peak with desired mass was detected. The reaction mixture was diluted with H₂O 8 mL and extracted with DCM (8 mL*4). The combined organic layers were dried over Na₂SO₄, filtered, and concentrated under reduced pressure to give a residue. The residue was purified by column chromatography (SiO₂, Petroleum ether/Ethyl acetate=100/1 to 0/1). 7 (50 mg, 84.89 μmol, 32% yield) was obtained as a yellow solid.

### General Procedure for Preparation of Compound 220 (8):

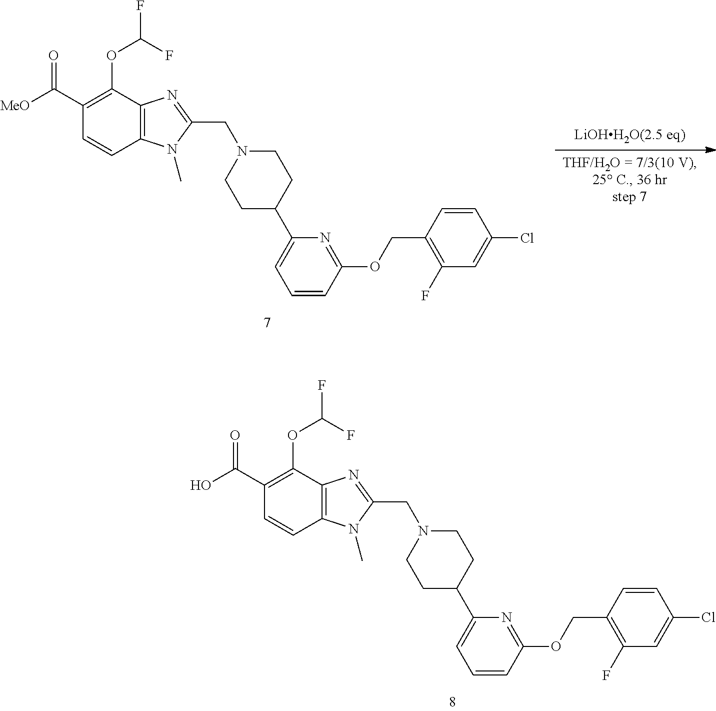

A mixture of 7 (45 mg, 76.40 μmol, 1 eq), LiOH·H₂O (4.81 mg, 114.60 μmol, 1.5 eq) in THF (0.7 mL) and H₂O (0.3 mL) was degassed and purged with N₂ for 3 times, and then the mixture was stirred at 25° C. for 12 hr under N₂ atmosphere. The mixture was added LiOH·H₂O (3.21 mg, 76.40 μmol, 1 eq), then the reaction was stirred at 25° C. for 24 hr under N₂ atmosphere. LCMS (RT=1.190 min) showed 7 was consumed completely and one main peak with desired mass was detected. The reaction mixture was filtered. The filtrate was purified by prep-HPLC (column: Waters Xbridge Prep OBD C₁₈ 150*40 mm*10 um; mobile phase: [H₂O (10 mM NH₄HCO₃)-ACN]; gradient: 20%-60% B over 8.0 min). 2-((4-(6-((4-Chloro-2-fluorobenzyl)oxy)pyridin-2-yl)piperidin-1-yl)methyl)-4-(difluoromethoxy)-1-methyl-1H-benzo[d]imidazole-5-carboxylic acid (18.36 mg, 31.93 μmol, 41% yield) was obtained as a white solid.

LCMS: RT=2.302 min, MS cal.: 575.0, [M+H]⁺=575.2

HPLC: RT=11.911 min, purity: 97.03%

¹H NMR (400 MHz, METHANOL-d₄) δ=7.81 (d, J=8.4 Hz, 1H), 7.61-7.55 (m, 1H), 7.51-7.43 (m, 2H), 7.33 (t, J=76 Hz, 1H), 7.22-7.15 (m, 2H), 6.83 (d, J=7.2 Hz, 1H), 6.63 (d, J=8.0 Hz, 1H), 5.40 (s, 2H), 4.00 (s, 2H), 3.98 (s, 3H), 3.13 (d, J=11.2 Hz, 2H), 2.77-2.61 (m, 1H), 2.52-2.37 (m, 2H), 1.96-1.82 (m, 4H)

### Example A263—Preparation of Compound 237. 2-((4-(6-((4-Chlorobenzofuran-7-yl)methoxy)pyridin-2-yl)piperidin-1-yl)methyl)-4-(difluoromethoxy)-1-methyl-1H-benzo[d]imidazole-6-carboxylic acid (Compound 237) (7)

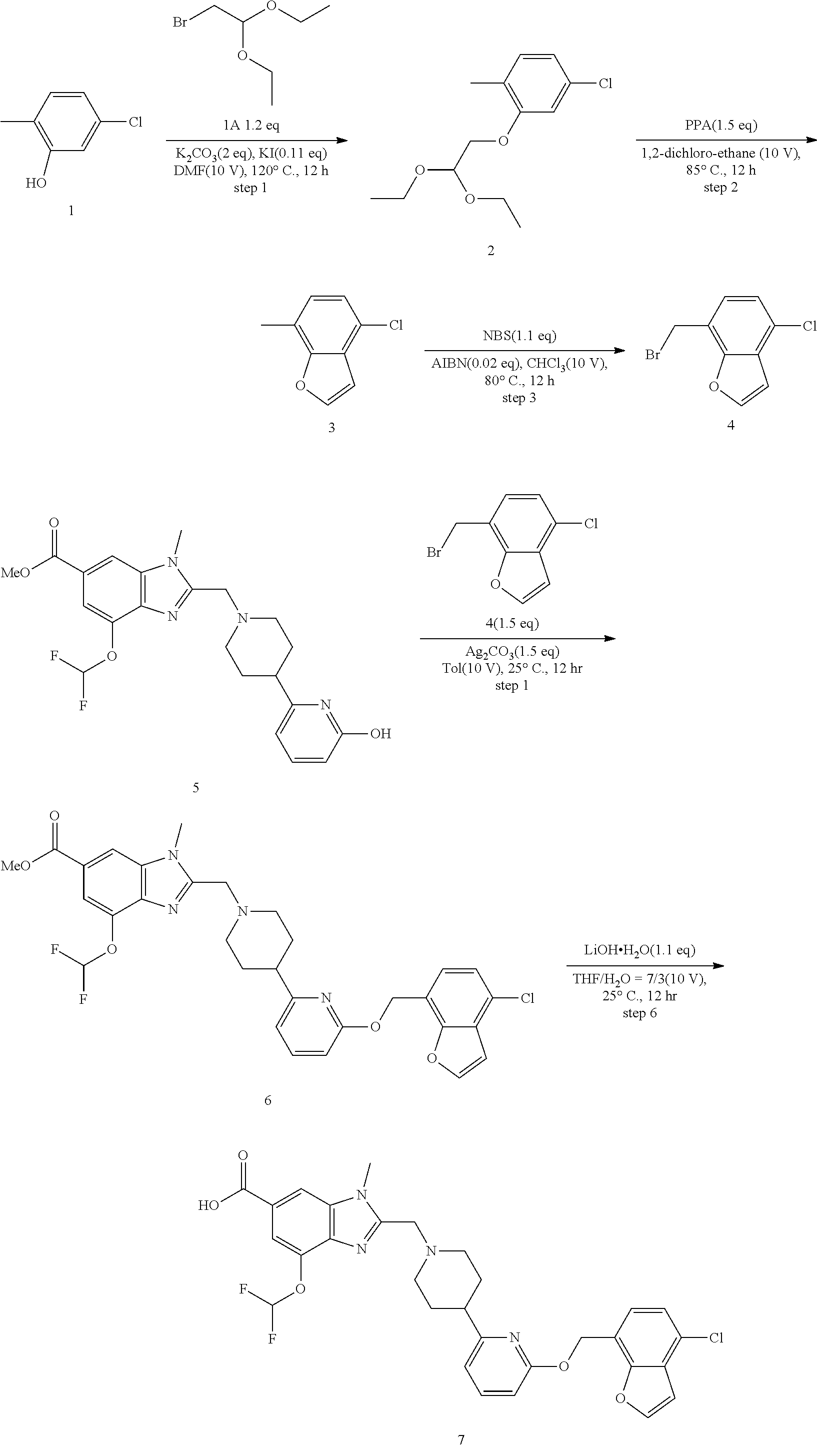

### General Procedure for Preparation of Intermediate 2:

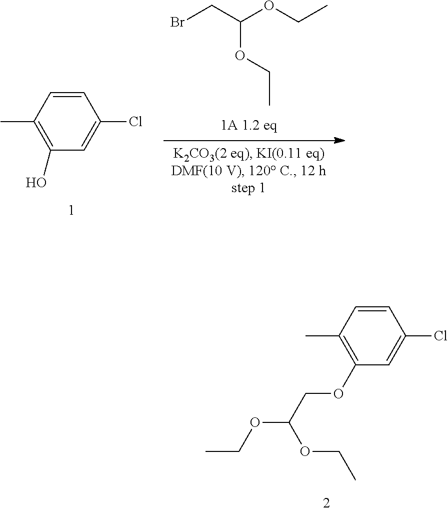

To a mixture of 1 (5 g, 35.07 mmol, 1 eq), 1A (8.29 g, 42.08 mmol, 6.33 mL, 1.2 eq), K₂CO₃ (9.69 g, 70.13 mmol, 2 eq) and KI (640.34 mg, 3.86 mmol, 0.11 eq) in DMF (50 mL) was stirred at 120° C. for 12 hours under N₂. The color changes to brown. TLC (SiO₂, PE:EA=5:1, R_{f}=0.70) showed the reaction was completed. The reaction mixture was quenched by addition water 100 mL at 25° C., and then extracted with EA 150 mL (50 mL*3). The combined organic layers were washed with brine 500 mL (100 mL*5), dried over Na₂SO₄, filtered and concentrated under reduced pressure to give a residue. The residue was purified by column chromatography (SiO₂, Petroleum ether/Ethyl acetate=20/1 to 15/1). 2 (7.6 g, 28.64 mmol, 81.67% yield) was obtained as light-yellow oil.

HPLC: RT=3.044 min, purity: 97.50%

¹H NMR (400 MHz, METHANOL-d4) δ=7.09 (d, J=8.0 Hz, 1H), 6.91 (d, J=1.6 Hz, 1H), 6.84 (dd, J=2.0, 8.0 Hz, 1H), 4.86-4.84 (m, 1H), 3.98 (d, J=5.2 Hz, 2H), 3.78 (qd, J=2.4, 7.2 Hz, 2H), 3.71-3.61 (m, 2H), 2.17 (s, 3H), 1.23 (t, J=7.2 Hz, 6H)

### General Procedure for Preparation of Intermediate 3:

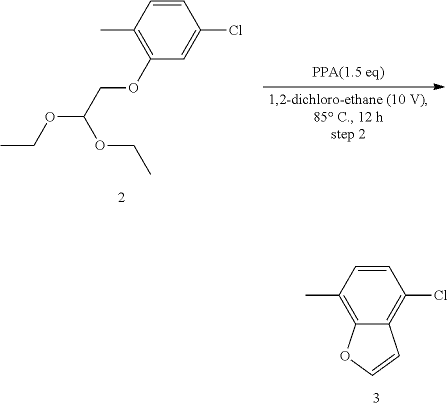 [SMILES: CCOC(COc1cc(Cl)ccc1C)OCC ; "1,2-dichloro-ethane (10 V),85° C., 12 hstep 2" ; "PPA(1.5 eq)" ; Cc1ccc(Cl)c2ccoc12]

To a mixture of 2 (4 g, 15.46 mmol, 1 eq) in DCE (64 mL) was added PPA (15.37 g, 38.65 mmol, 85% purity, 2.5 eq). The mixture was stirred at 85° C. for 12 hours. The color changes to brown. TLC (SiO₂, PE:EA=10:1, R_{f}=0.80) showed the reaction was completed. The reaction mixture was quenched by addition water 300 mL, and then extracted with EA 600 mL (200 mL*3). The combined organic layers were washed with brine 500 mL, dried over Na₂SO₄, filtered, and concentrated under reduced pressure to give residue. The residue was purified by column chromatography (SiO₂, Petroleum ether/Ethyl acetate=1/0 to 99/1). 3 (1.7 g, 9.85 mmol, 63.75% yield) was obtained as brown oil.

HPLC: RT=2.787 min, purity: 96.58%

¹H NMR (400 MHz, METHANOL-d4) δ=7.81 (t, J=1.6 Hz, 1H), 7.14-7.11 (m, 1H), 7.08-7.04 (m, 1H), 6.87 (dd, J=1.2, 2.0 Hz, 1H), 2.47 (s, 3H)

### General Procedure for Preparation of Intermediate 4:

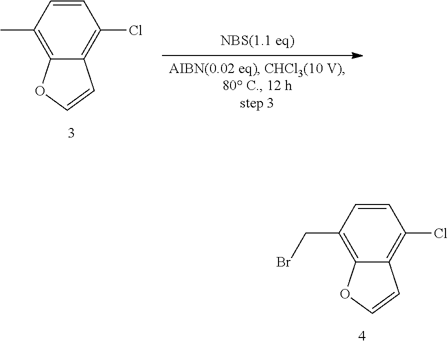

To a mixture of 3 (500 mg, 3.00 mmol, 1 eq), NBS (587.57 mg, 3.30 mmol, 1.1 eq) and AIBN (6.16 mg, 37.51 μmol, 0.0125 eq) in CCl₄ (10 mL) was stirred at 80° C. for 12 hours under N₂. The color changes to orange. TLC (SiO₂, PE:EA=0:1, R_{f}=0.50) showed the reaction was completed. The reaction mixture was quenched by addition water 10 mL, and then extracted with EA 30 mL (10 mL*3). The combined organic layers were washed with brine 30 mL, dried over Na₂SO₄, filtered, and concentrated under reduced pressure to give a residue. The residue was purified by prep-TLC (SiO₂, PE:EA=0:1, R_{f}=0.45). 4 (106 mg, 409.23 μmol, 13.64% yield) was obtained as yellow oil.

HPLC: RT=2.735 min, purity: 94.78%

¹H NMR (400 MHz, METHANOL-d4) δ=7.92 (d, J=2.0 Hz, 1H), 7.35-7.31 (m, 1H), 7.26-7.22 (m, 1H), 6.96 (d, J=2.4 Hz, 1H), 4.82 (s, 2H)

### General Procedure for Preparation of Intermediate 5:

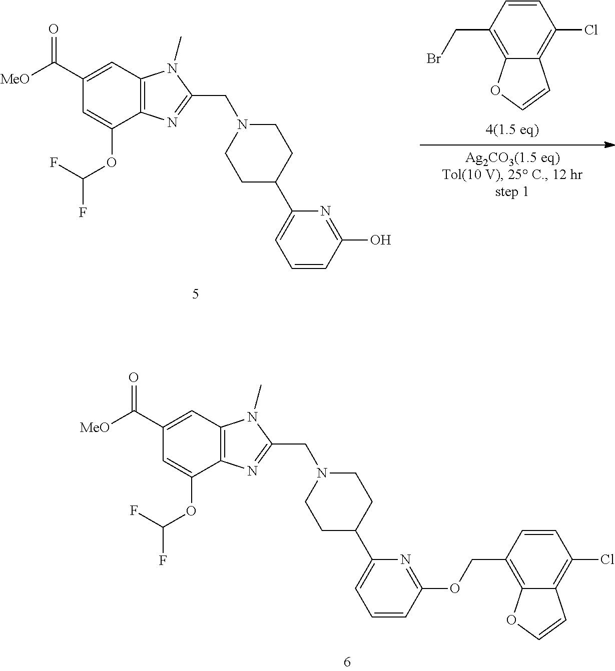

To a mixture of 5 (90 mg, 201.59 μmol, 1 eq), 4 (74.24 mg, 302.39 μmol, 1.5 eq) and Ag₂CO₃ (111.18 mg, 403.18 μmol, 18.29 μL, 2 eq) in Tol. (1 mL) was stirred at 100° C. for 12 hours under N₂. The color changes to black. TLC (SiO₂, PE:EA=2:1, R_{f}=0.50) showed the reaction was completed. The reaction mixture was quenched by addition water 5 mL, and then extracted with EA 15 mL (5 mL*3). The combined organic layers were washed with brine 10 mL, dried over Na₂SO₄, filtered, and concentrated under reduced pressure to give a residue. The residue was purified by prep-TLC (SiO₂, PE:EA=2:1, R_{f}=0.45). 6 (80 mg, 125.71 μmol, 62.36% yield) was obtained as yellow oil.

HPLC: RT=3.506 min, purity: 96.02%)

¹H NMR (400 MHz, METHANOL-d4) δ=8.13 (d, J=1.2 Hz, 1H), 7.87 (d, J=2.4 Hz, 1H), 7.68 (s, 1H), 7.56 (t, J=7.6 Hz, 1H), 7.27 (t, J=74.0 Hz, 1H), 7.36 (d, J=8.4 Hz, 1H), 7.22 (d, J=8.0 Hz, 1H), 6.92 (d, J=2.4 Hz, 1H), 6.81 (d, J=7.2 Hz, 1H), 6.63 (d, J=8.4 Hz, 1H), 5.64 (s, 2H), 4.02 (s, 3H), 3.96 (s, 3H), 3.90 (s, 2H), 3.04-2.96 (m, 2H), 2.68-2.57 (m, 1H), 2.36-2.26 (m, 2H), 1.88-1.78 (m, 4H)

### General Procedure for Preparation of Compound 237 (7):

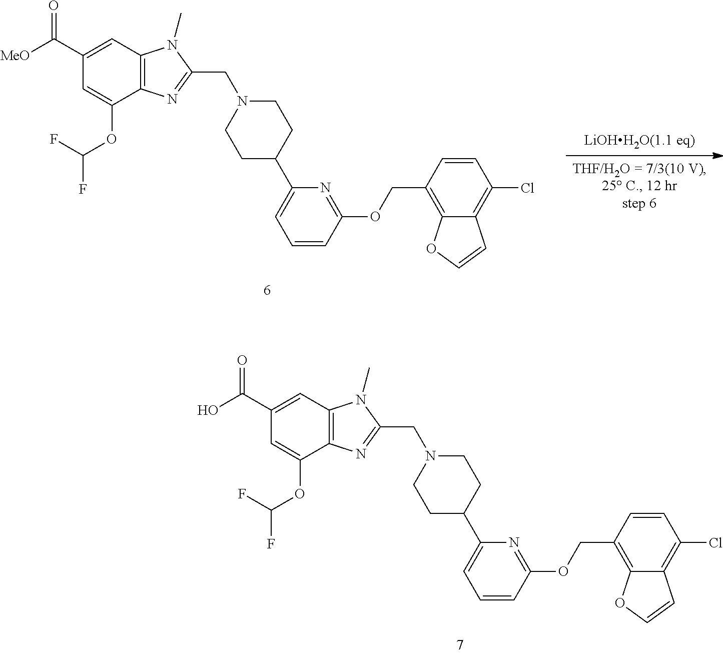

A mixture of 6 (80 mg, 130.93 μmol, 1 eq), in THF (0.7 mL) and H₂O (0.3 mL) was added LiOH·H₂O (8.24 mg, 196.39 μmol, 1.5 eq). The mixture was stirred at 25° C. for 12 hours. The color changes to yellow. LCMS showed 84% of desired product was detected. The mixture was concentrated in vacuum. The residue was purified by prep-HPLC (column: Waters Xbridge Prep OBD C18 150*40 mm*10 um; mobile phase: [H₂O (10 mM NH₄HCO₃)-ACN]; gradient: 20%-70% B over 8.0 min). 2-((4-(6-((4-Chlorobenzofuran-7-yl)methoxy)pyridin-2-yl)piperidin-1-yl)methyl)-4-(difluoromethoxy)-1-methyl-1H-benzo[d]imidazole-6-carboxylic acid (28.35 mg, 46.90 μmol, 35.82% yield, 98.77% purity) was obtained as a white solid.

LCMS: RT=2.829 min, MS cal.: 596.2, [M+H]⁺=597.2.

HPLC: RT=12.268 min, purity: 98.77%

¹H NMR (400 MHz, DMSO-d6) δ=8.16 (d, J=2.0 Hz, 1H), 8.07 (d, J=0.8 Hz, 1H), 7.65-7.59 (m, 1H), 7.64 (t, J=74.0 Hz, 1H), 7.52 (s, 1H), 7.42 (d, J=7.6 Hz, 1H), 7.33 (d, J=7.6 Hz, 1H), 7.03 (d, J=2.0 Hz, 1H), 6.86 (d, J=7.2 Hz, 1H), 6.67 (d, J=8.0 Hz, 1H), 5.60 (s, 2H), 3.96 (s, 3H), 3.85 (s, 2H), 2.93 (d, J=10.8 Hz, 2H), 2.62-2.54 (m, 1H), 2.21 (t, J=10.0 Hz, 2H), 1.80-1.62 (m, 4H)

### Example A264—Preparation of Compound 254. 4-(Difluoromethoxy)-2-((4-(6-((4-fluorobenzyl)oxy)pyridin-2-yl)piperidin-1-yl)methyl)-1-methyl-1H-benzo[d]imidazole-6-carboxylic acid (Compound 254) (3)

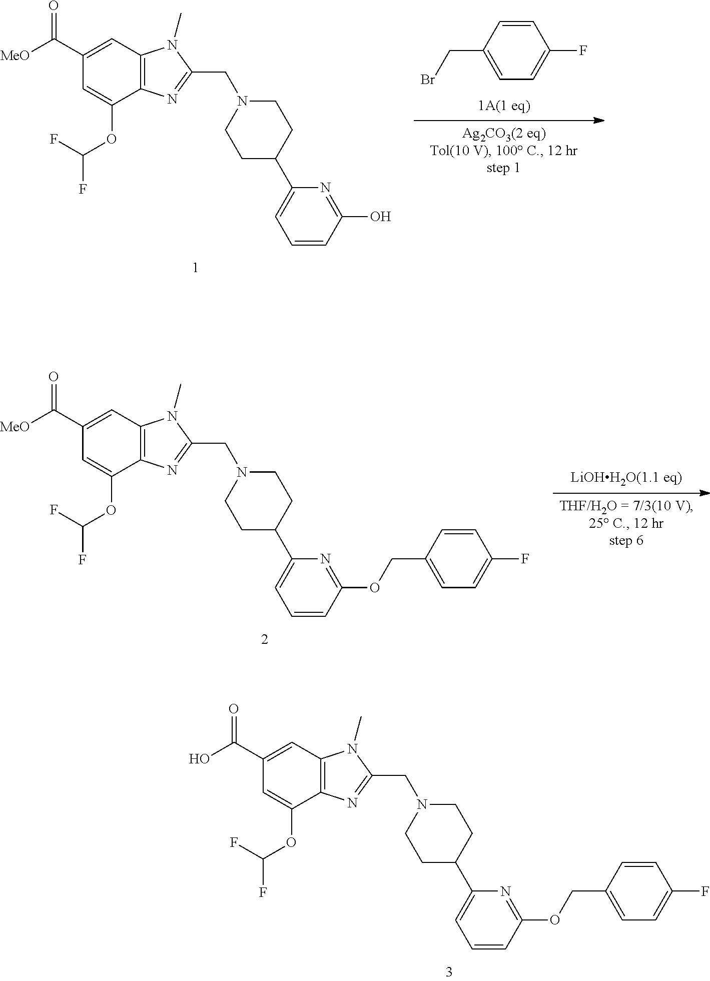

### General Procedure for Preparation of Intermediate 2:

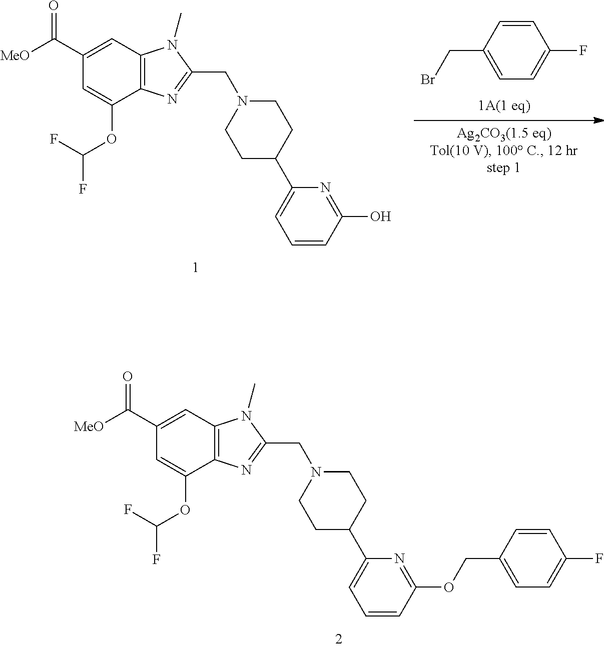

To a mixture of 1 (100 mg, 223.99 μmol, 1 eq) and 1A (42.34 mg, 223.99 μmol, 27.60 μL, 1 eq) in toluene (1 mL) was added Ag₂CO₃ (92.64 mg, 335.99 μmol, 15.25 μL, 1.5 eq) in one portion at 20° C. under N₂. The mixture was stirred at 100° C. for 12 hours. The color of the solution changed to black. LC-MS showed 1 was consumed completely and one main peak with desired mass was detected. Filtrated, the mixture was concentrated in vacuum. The crude product was purified by prep-TLC (Petroleum ether:Ethyl acetate=2:1, R_{f}=0.35). 2 (90 mg, 162.29 μmol, 72.45% yield) as a white solid.

LCMS: RT=0.469 min, MS cal.: 554.3, [M+H]⁺=555.3

1H NMR (400 MHz, DMSO-d6) δ=8.13 (d, J=1.2 Hz, 1H), 7.68 (s, 1H), 7.56 (t, J=7.6 Hz, 1H), 7.47-7.43 (m, 2H), 7.26 (t, J=84.0 Hz, 1H), 7.07 (d, J=9.2 Hz, 2H), 6.80 (d, J=7.2 Hz, 1H), 6.61 (d, J=8.4 Hz, 1H), 5.33 (s, 2H), 4.03 (s, 3H), 3.96 (s, 3H), 3.92 (s, 2H), 3.03 (br d, J=12.0 Hz, 2H), 2.69-2.60 (m, 1H), 2.37-2.30 (m, 2H), 1.91-1.85 (m, 4H)

### General Procedure for Preparation of Compound 254 (3):

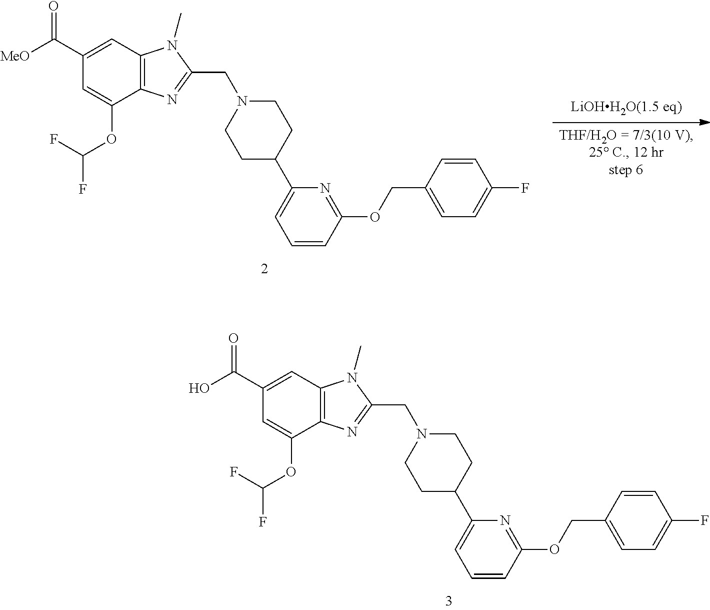

To a mixture of 2 (90 mg, 162.29 μmol, 1 eq) in THF (0.7 mL) and H₂O (0.3 mL) was added LiOH·H₂O (10.21 mg, 243.44 μmol, 1.5 eq) in one portion at 20° C. under N₂. The mixture was stirred at 20° C. for 12 hours. The color of the solution changed to yellow. LC-MS showed 2 was consumed completely and one main peak with desired mass was detected. The mixture was filtered and concentrated in vacuum. The residue was purified by prep-HPLC (column: Waters Xbridge Prep OBD C₁₈ 150*40 mm*10 um; mobile phase: [H₂O (10 mM NH₄HCO₃)-ACN]; gradient: 30%-60% B over 8.0 min). 4-(Difluoromethoxy)-2-((4-(6-((4-fluorobenzyl)oxy)pyridin-2-yl)piperidin-1-yl)methyl)-1-methyl-1H-benzo[d]imidazole-6-carboxylic acid (29.73 mg, 55.00 μmol, 33.89% yield) as a white solid.

LCMS: RT=2.870 min, MS cal.: 540.2, [M+H]⁺=541.3

HPLC: RT=11.051 min, purity: 94.91%

¹H NMR (400 MHz, METHANOL-d4) δ=8.06 (s, 1H), 7.64-7.59 (m, 1H), 7.63 (t, J=74.0 Hz, 1H), 7.53-7.49 (m, 2H), 7.48 (d, J=5.6 Hz, 1H), 7.18 (t, J=8.8 Hz, 2H), 6.85 (d, J=7.2 Hz, 1H), 6.65 (d, J=8.2 Hz, 1H), 5.31 (s, 2H), 3.96 (s, 3H), 3.85 (s, 2H), 2.95 (br d, J=10.8 Hz, 2H), 2.64-2.57 (m, 1H), 2.22 (br t, J=10.4 Hz, 2H), 1.84-1.78 (m, 2H), 1.77-1.66 (m, 2H)

### Example A265—Preparation of Compound 142. 2-(((1s,4s)-4-(6-((4-Cyano-2-fluorobenzyl)oxy)pyridin-2-yl)cyclohexyl)(methyl)amino)-4-(difluoromethoxy)-1-methyl-1H-benzo[d]imidazole-6-carboxylic acid (Compound 142) (5)

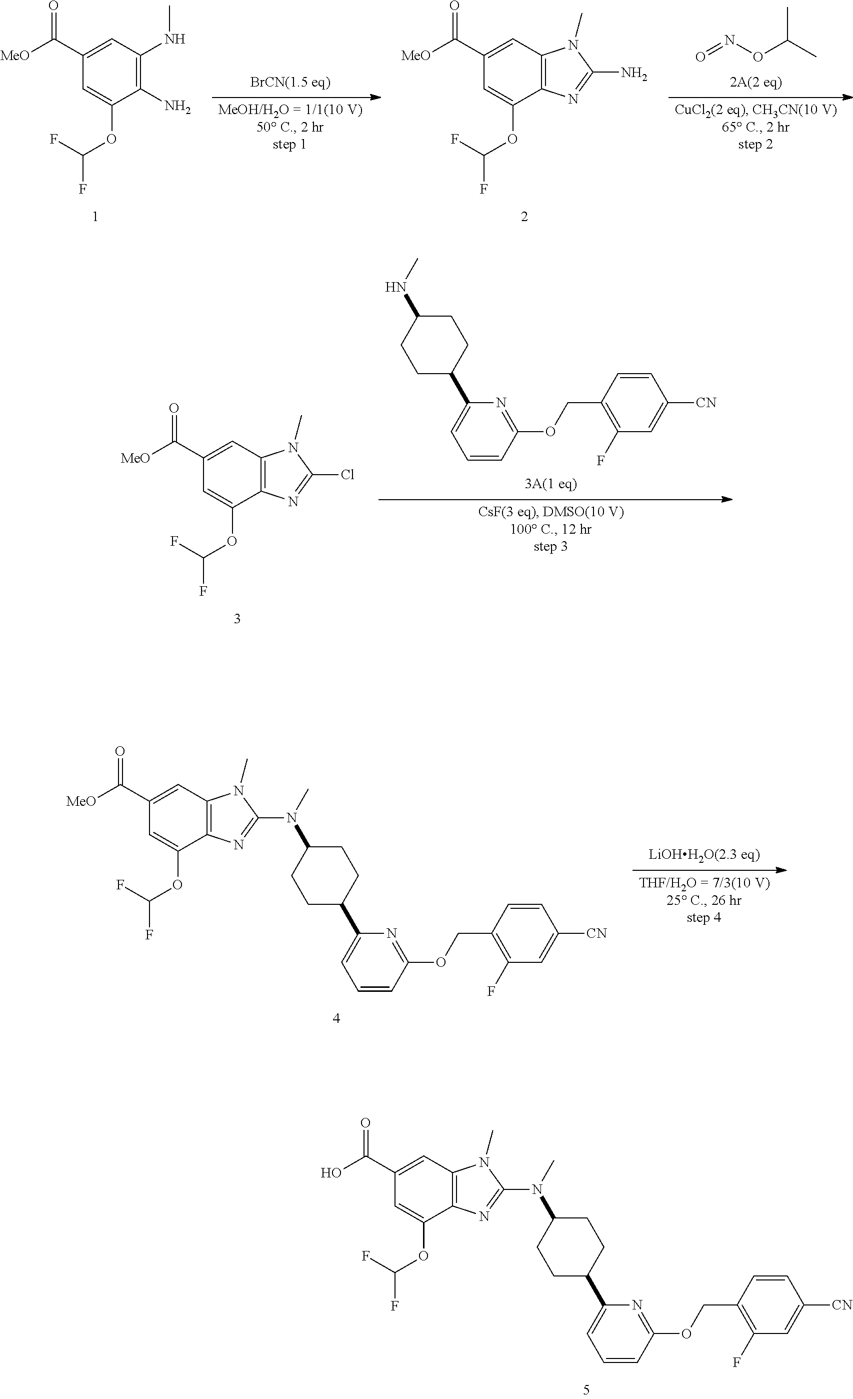

### General Procedure for Preparation of Intermediate 2:

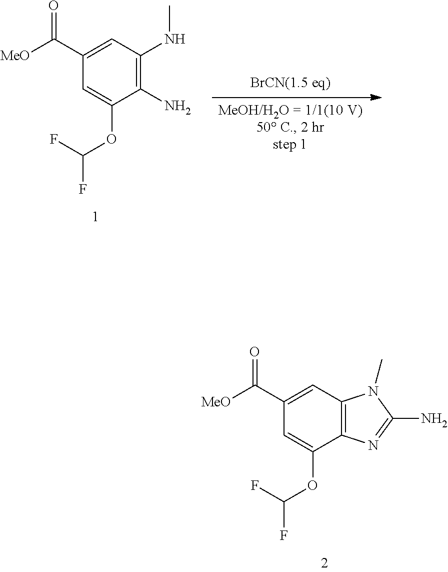

Equip a 25 mL three-necked round bottom flask, thermometer, N₂ balloon. MeOH (5 mL) and H₂O (5 mL) was charged to the 25 mL three-necked round bottom flask, then 1 (500 mg, 2.03 mmol, 1 eq) was added to the mixture at 20° C. BrCN (322.66 mg, 3.05 mmol, 223.60 μL, 1.5 eq) was added dropwise to the reaction mixture at 20° C. After the addition, the mixture was stirred at 50° C. for 2 hr. LCMS showed 1 was consumed completely and one main peak with desired mass was detected. The reaction mixture was adjusted to pH=8 with 1M NaHCO₃ at 20° C., and then diluted with H₂O (10 mL) and extracted with EtOAc (20 mL*2). The combined organic layers were washed with brine 20 mL, dried over Na₂SO₄, filtered and concentrated under reduced pressure to give a residue. 2 (530 mg, 1.93 mmol, 95.26% yield) was obtained as a brown solid.

LCMS: RT=0.301 min, MS cal.: 271.1/272.1, [M+H]⁺=272.0/273.0

¹HNMR (400 MHz, CHLOROFORM-d) δ=7.74-7.72 (m, 1H), 7.68-7.66 (m, 1H), 7.07 (t, J=74.8 Hz, 1H), 3.94 (s, 3H), 3.62-3.62 (m, 1H), 3.62 (s, 2H)

### General Procedure for Preparation of Intermediate 3:

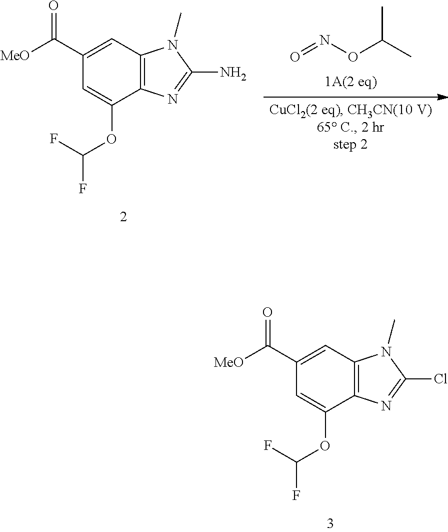

To a solution of 2 (280 mg, 1.03 mmol, 1 eq) in CH₃CN (3 mL) was added CuCl₂ (277.61 mg, 2.06 mmol, 2 eq) and 1A (212.92 mg, 2.06 mmol, 245.58 μL, 2 eq). The mixture was stirred at 65° C. for 2 hr. LCMS showed 2 was consumed completely. Several new peaks were shown on LCMS and 68% of desired 3 was detected. The reaction mixture was diluted with H₂O 10 mL and extracted with EtOAc 20 mL (10 mL*2). The combined organic layers were washed with brine 10 mL, dried over Na₂SO₄, filtered and concentrated under reduced pressure to give a residue. The residue was purified by column chromatography (SiO₂, PE/EtOAc=1/0 to 4/1). 3 (190 mg, 647.17 μmol, 62.69% yield) was obtained as a yellow solid.

LCMS: RT=0.459 min, MS cal.: 290.2/292.0, [M+H]⁺=290.9/292.9

¹HNMR (400 MHz, CHLOROFORM-d) δ=7.94 (s, 1H), 7.76 (s, 1H), 7.26 (t, J=72 Hz, 1H), 3.98 (s, 3H), 3.87 (s, 3H)

### General Procedure for Preparation of Intermediate 4:

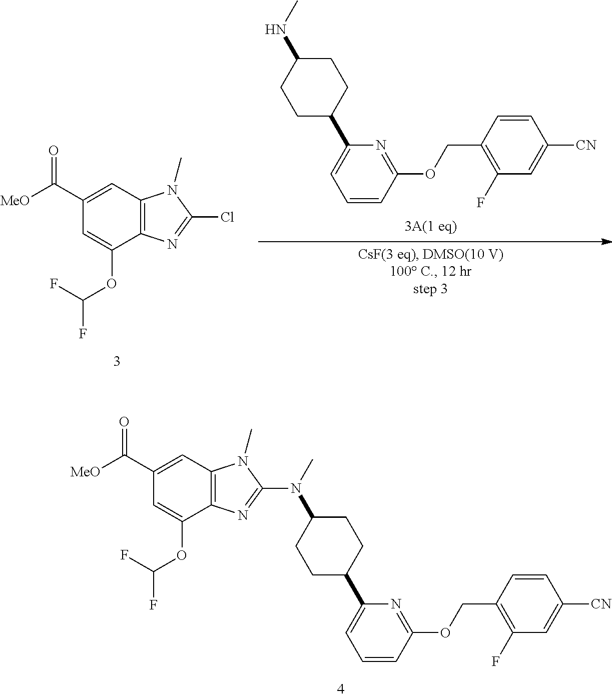

To a solution of 3 (100 mg, 344.06 μmol, 1 eq) in DMSO (1.5 mL) was added CsF (156.79 mg, 1.03 mmol, 3 eq) and 3A (116.77 mg, 344.06 μmol, 1 eq). The mixture was stirred at 100° C. for 12 hr. LCMS showed 3 was consumed completely. Several new peaks were shown on LCMS and 38% of desired 4 was detected. The reaction mixture was diluted with H₂O 5 mL and extracted with EtOAc 10 mL (5 mL*2). The combined organic layers were washed with brine 5 mL, dried over Na₂SO₄, filtered, and concentrated under reduced pressure to give a residue. The residue was purified by column chromatography (SiO₂, PE/EtOAc=1/0 to 1/1). 4 (70 mg, 109.67 μmol, 31.88% yield) was obtained as a white solid.

LCMS: RT=0.563 min, MS cal.: 593.2/594.2, [M+H]⁺=594.2/595.2

### General Procedure for Preparation of Compound 142 (5):

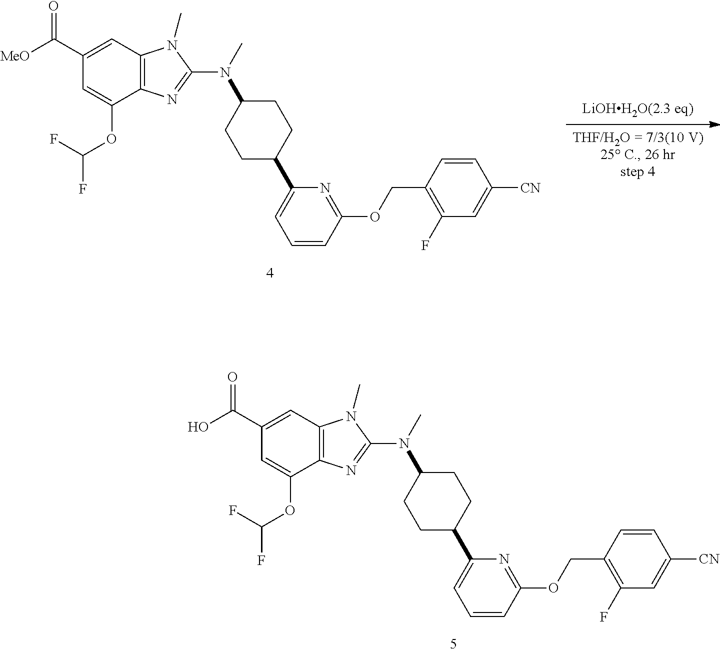

To a solution of 4 (70 mg, 117.93 μmol, 1 eq) in THF (0.7 mL) and H₂O (0.3 mL) was added

LiOH·H₂O (7.42 mg, 176.89 μmol, 1.5 eq). The mixture was stirred at 25° C. for 2 hr. The mixture was added another LiOH·H₂O (1.48 mg, 35.38 μmol, 0.3 eq). The mixture was stirred at 25° C. for 12 hr. The mixture was added LiOH·H₂O (2.47 mg, 58.96 μmol, 0.5 eq). The mixture was stirred at 25° C. for 12 hr. LCMS showed 36% of 4 remained and 56% of desired product was detected. The mixture was added CH₃CN 0.2 mL and purified by prep-HPLC (neutral condition: column: Waters Xbridge Prep OBD C18 150*40 mm*10 um; mobile phase: [H₂O (10 mM NH₄HCO₃)-ACN]; gradient: 30%-60% B over 8.0 min). The residue was purified by prep-HPLC (FA condition: column: column: Phenomenex Luna C18 100*30 mm*3 um; mobile phase: [H₂O (0.2% FA)-ACN]; gradient: 25%-65% B over 8.0 min). 2-(((1s,4s)-4-(6-((4-Cyano-2-fluorobenzyl)oxy)pyridin-2-yl)cyclohexyl)(methyl)amino)-4-(difluoromethoxy)-1-methyl-1H-benzo[d]imidazole-6-carboxylic acid (8.6 mg, 14.39 μmol, 12.21% yield) was obtained as a white solid.

LCMS: RT=2.793 min, MS cal.: 579.2/580.2, [M+H]⁺=580.2/581.2

HPLC: RT=12.778 min

¹H NMR (400 MHz, DMSO-d₆) δ=7.89 (s, 1H), 7.70-7.52 (m, 5H), 7.21 (t, J=74.8 Hz, 1H), 6.96 (d, J=7.6 Hz, 1H), 6.72 (d, J=8.0 Hz, 1H), 5.57 (s, 2H), 3.74 (s, 3H), 3.72-3.65 (m, 1H), 2.91 (m, 1H), 2.81 (s, 3H), 2.27-2.18 (m, 2H), 2.01 (br s, 2H), 1.81-1.72 (m, 2H), 1.67 (td, J=3.6, 12.8 Hz, 2H)

### Example A266—Preparation of Compound 180. 2-((4-(6-((4-Cyano-2-fluorobenzyl)oxy)pyridin-2-yl)piperidin-1-yl)methyl)-4-(1,1-difluoroethoxy)-1-methyl-1H-benzo[d]imidazole-6-carboxylic acid (Compound 180) (9)

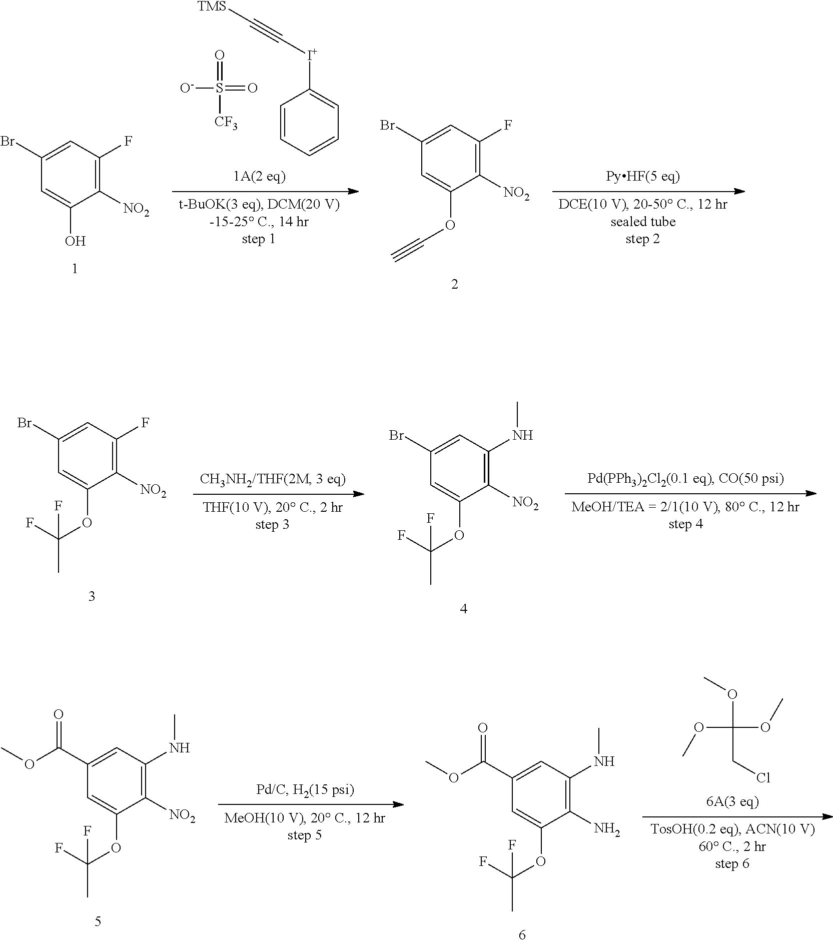

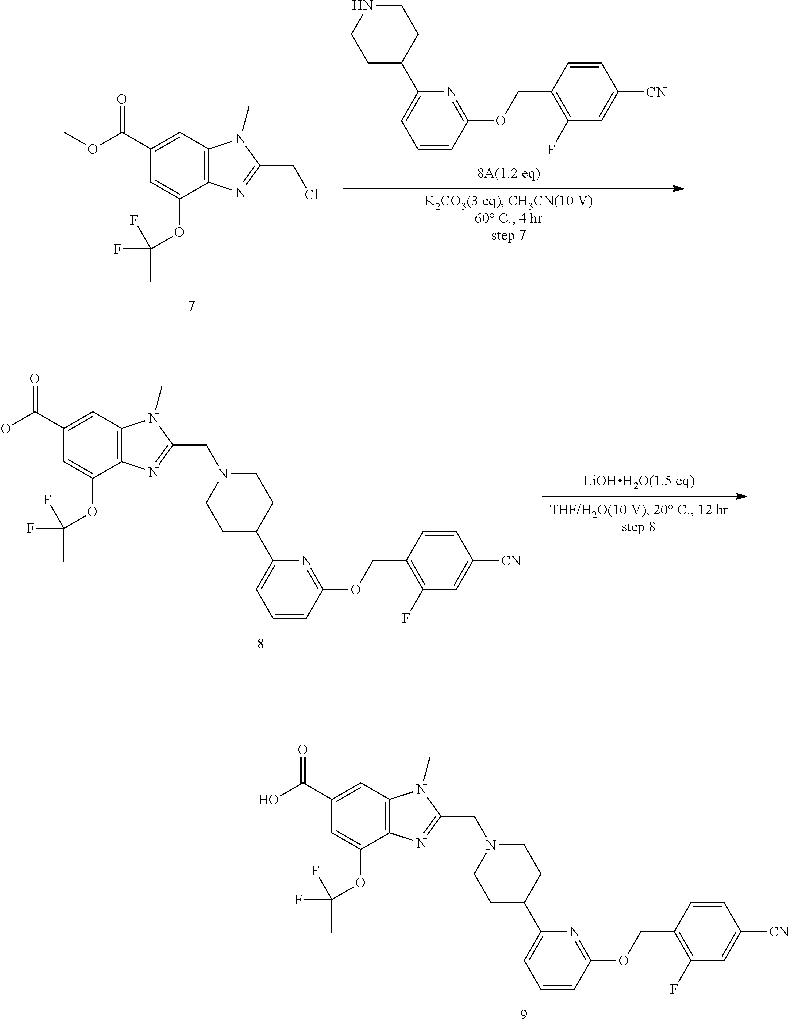

### General Procedure for Preparation of Intermediate 2:

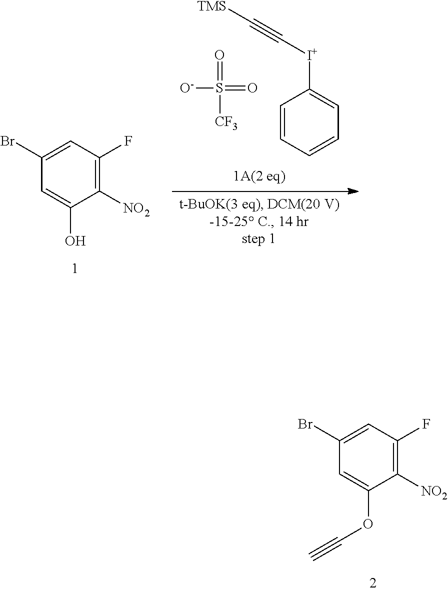

To a solution of 1A (2.95 g, 6.36 mmol, 1.5 eq) in DCM (10 mL) was added t-BuOK (1 M, 8.47 mL, 2 eq) at −20° C., the mixture was stirred at −20° C. for 0.5 hr. Then it was added 1 (1 g, 4.24 mmol, 1 eq) in DCM (10 mL) at −20° C. The mixture was stirred at 20° C. for 12 hr. TLC (Petroleum ether:Ethyl acetate-7:1, product Rf=0.55) indicated ˜50% of 1 was remained, and one major new spot with lower polarity was detected. t-BuOK (1 M, 4.24 mL, 1 eq) was added at −20° C., the mixture was stirred at −20° C. for 0.5 hr, then added 1A (983.73 mg, 2.12 mmol, 0.5 eq) in DCM (10 mL) at −20° C. The mixture was stirred at 20° C. for 2 hr. LCMS showed ˜22% compound 1 was remained, and ˜64% of desired mass was detected. The reaction mixture was partitioned between H₂O 30 mL and DCM 45 mL. The organic phase was separated, washed with brine 20 mL, dried over Na₂SO₄, filtered, and concentrated under reduced pressure to give a residue. The residue was purified by column chromatography (SiO₂, Petroleum ether/Ethyl acetate=100/0). 2 (200 mg, 769.18 μmol, 18% yield) was obtained as a brown oil.

LCMS: RT=0.820 min, MS cal.: 258.9, 260.9, [M−25]⁺=234.0, 236.0

1H NMR (400 MHz, CHLOROFORM-d) δ=7.72 (t, J=1.6 Hz, 1H), 7.32-7.30 (dd, J=8.4, 1.6 Hz, 1H), 2.36 (s, 1H)

### General Procedure for Preparation of Intermediate 3:

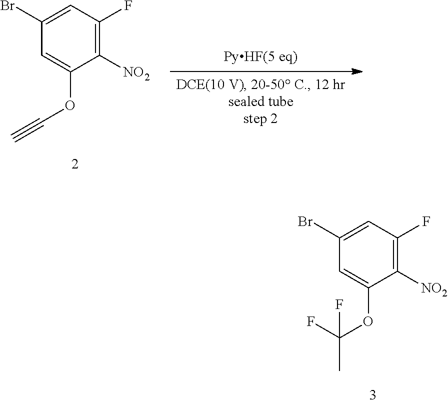 [SMILES: C#COc1cc(Br)cc(F)c1[N+](=O)[O-] ; "DCE(10 V), 20-50° C., 12 hrsealed tubestep 2" ; "Py·HF(5 eq)" ; CC(F)(F)Oc1cc(Br)cc(F)c1[N+](=O)[O-]]

Equip a 30 mL sealed tube, DCE (2 mL) was charged to the 30 mL sealed tube, then compound 2 (200 mg, 769.18 μmol, 1 eq), pyridine; hydrofluoride (544.50 mg, 3.85 mmol, 1.73 mL, 70% purity, 5 eq) was added at 20° C. under N₂, the mixture was stirred at 50° C. (15 Psi) for 12 hr. TLC (Petroleum ether:Ethyl acetate=7:1, product R_{f}=0.58) indicated 2 was consumed completely and one new spot formed. The reaction was clean according to TLC. The reaction mixture was poured into 1M HCl (5 mL) and extracted with EtOAc (15 mL). The combined organic layer was washed with brine 10 mL, dried over Na₂SO₄, filtered, and concentrated in vacuum to give a crude product. The residue was purified by column chromatography (SiO₂, Petroleum ether/Ethyl acetate=100/0). 3 (70 mg, 233.31 μmol, 30% yield) was obtained as a white solid.

LCMS: RT=0.852 min, MS cal.: 298.9, 300.9, [M−44]⁺=255.3, 256.3

¹H NMR (400 MHz, CHLOROFORM-d) δ=7.48 (s, 1H), 7.34-7.32 (dd, J=8.4 Hz, 1H), 1.97 (t, J=14.0 Hz, 3H).

### General Procedure for Preparation of Intermediate 4:

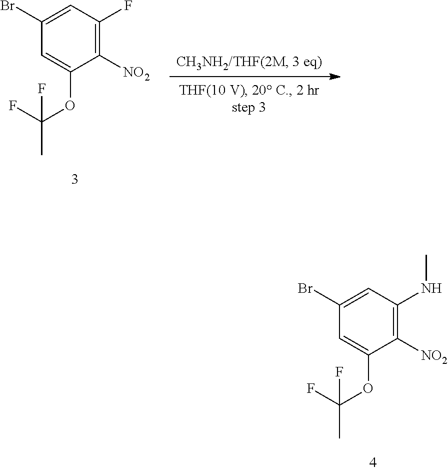 [SMILES: CC(F)(F)Oc1cc(Br)cc(F)c1[N+](=O)[O-] ; "CH3NH2/THF(2M, 3 eq)" ; "THF(10 V), 20° C., 2 hrstep 3" ; CNc1cc(Br)cc(OC(C)(F)F)c1[N+](=O)[O-]]

Equip a 50 mL round bottom flask, THF (4 mL) was charged to the round bottom flask, then 3 (370 mg, 1.23 mmol, 1 eq) was added to the mixture at 20° C. At 20° C. (inner temperature), methanamine (2 M, 1.85 mL, 3 eq) was added to the reaction mixture at 20° C. under N₂ atmosphere. After the addition, the suspension was degassed and purged with N₂ for 3 times, and then the mixture was stirred under N₂ atmosphere at 20° C. for 2 hr. LC-MS showed 3 was consumed completely and one main peak with desired mass was detected. The mixture quenched by addition 1M FA (10 mL) at 20° C., and then was diluted with H₂O (10 mL), extracted with EtOAc (10 mL*3). The combined organic layer was washed with 10 mL brine, dried over Na₂SO₄, filtered and the filtrate was concentrated to give a crude product. The residue was purified by column chromatography (SiO₂, Petroleum ether/Ethyl acetate=99/1). 4 (200 mg, 642.92 μmol, 52% yield) was obtained as a brown solid.

LCMS: RT=0.843 min, MS cal.: 310.0, 312.0, [M−H]⁺=309.1, 311.1

¹H NMR (400 MHz, CHLOROFORM-d) δ=6.84-6.82 (dd, J=6.4, 1.6 Hz, 2H), 6.40 (br, 1H), 2.94 (d, J=4.4 Hz, 3H), 1.95 (t, J=13.6 Hz, 3H)

### General Procedure for Preparation of Intermediate 5:

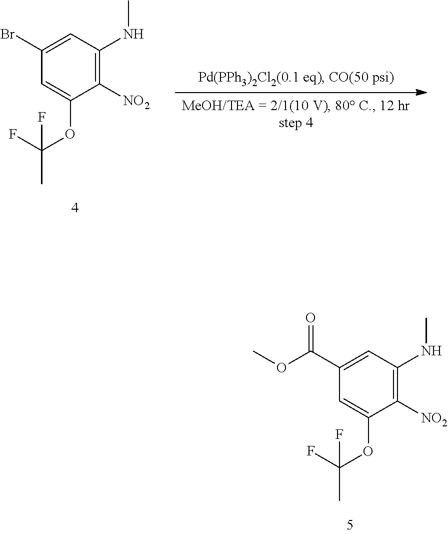

Equip a 35 mL hydrogenated bottle, MeOH (1.5 mL) and TEA (0.5 mL) was charged to 35 mL hydrogenated bottle, then 4 (200 mg, 642.92 μmol, 1 eq), Pd(PPh₃)₂Cl₂ (45.13 mg, 64.29 μmol, 0.1 eq) was added to the mixture at 20° C. After the addition, the mixture was degassed and purged with CO (50 Psi) 3 times. The mixture was stirred at 80° C. for 12 hr. TLC (Petroleum ether/Ethyl acetate=7/1, product R_{f}=0.23) indicated 4 was consumed completely and many new spots formed. The reaction mixture was diluted with H₂O 15 mL and extracted with EtOAc (10 mL*3). The combined organic layer was washed with brine 15 mL, dried over Na₂SO₄, filtered, and concentrated under reduced pressure to give a residue. The residue was purified by column chromatography (SiO₂, Petroleum ether/Ethyl acetate=94/6 to 78/22) 5 (100 mg, 344.57 μmol, 53% yield) was obtained as a brown oil.

¹H NMR (400 MHz, CHLOROFORM-d) δ=7.36 (d, J=1.2 Hz, 1H), 7.30 (d, J=1.2 Hz, 1H), 6.01 (br, 1H), 3.95 (s, 3H), 3.00 (s, 3H), 1.96 (t, J=13.6 Hz, 3H)

### General Procedure for Preparation of Intermediate 6:

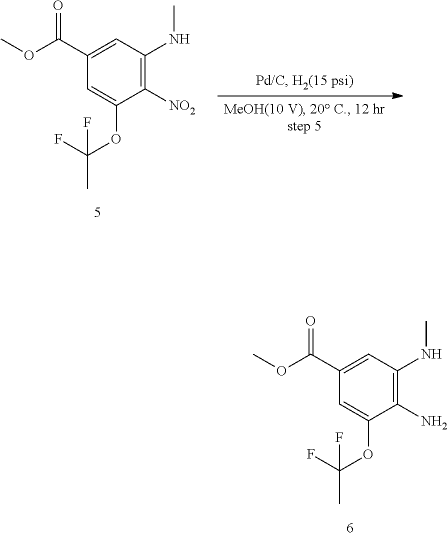

Equip a 10 mL round bottom flask, MeOH (1 mL) was charged to the round bottom flask, then Pd/C (50 mg) was added to the mixture at 20° C. 5 (100 mg, 344.57 μmol, 1 eq) in MeOH (1 mL) was added to the reaction mixture at 20° C. under N₂ atmosphere. After the addition, the suspension was degassed and purged with H₂ for 3 times, and then the mixture was stirred under H₂ (15 Psi) atmosphere at 20° C. for 12 hr. LC-MS showed 5 was consumed completely and one main peak with desired mass was detected. The suspension was filtered with celite, and the filter cake was washed with EtOAc (15 mL). The combined filtrates were concentrated to dryness. 6 (60 mg, 230.56 μmol, 67% yield) was obtained as a colourless oil.

LCMS: RT=0.683 min, MS cal.: 260.1, 261.1, [M−H]⁺=259.1, 260.1

¹H NMR (400 MHz, DMSO-d6) δ=7.12 (d, J=1.6 Hz, 1H), 6.84 (d, J=1.6 Hz, 1H), 5.29 (s, 2H), 5.16-5.06 (m, 1H), 3.75 (s, 3H), 2.76 (d, J=4.8 Hz, 3H), 1.99 (t, J=13.6 Hz, 3H)

### General Procedure for Preparation of Intermediate 7:

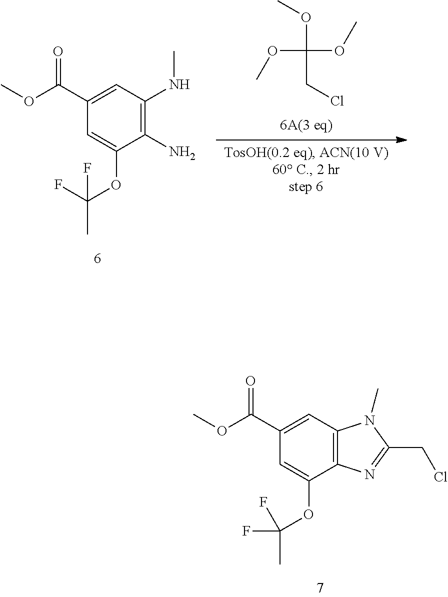

To a solution of 6 (90 mg, 345.84 μmol, 1 eq) in ACN (1 mL) was added TosOH (11.91 mg, 69.17 μmol, 0.2 eq) 2-chloro-1,1,1-trimethoxy-ethane (160.39 mg, 1.04 mmol, 139.84 μL, 3 eq). The mixture was stirred at 60° C. for 2 hr. LC-MS showed 6 was consumed completely and the desired mass was detected as a main peak. The reaction mixture was concentrated under reduced pressure to remove ACN. The residue was purified by column chromatography (SiO₂, Petroleum ether/Ethyl acetate=7/3). 7 (100 mg, 313.77 μmol, 45% yield) was obtained as a yellow solid.

LCMS: RT=0.437 min, MS cal.: 318.1, 320.1, [M+H]⁺=319.0, 321.0

¹H NMR (400 MHz, CHLOROFORM-d) δ=8.01 (d, J=1.2 Hz, 1H), 7.93 (d, J=1.2 Hz, 1H), 4.89 (s, 2H), 3.98 (s, 3H), 3.95 (s, 3H), 2.10 (t, J=13.4 Hz, 3H)

### General Procedure for Preparation of Intermediate 8:

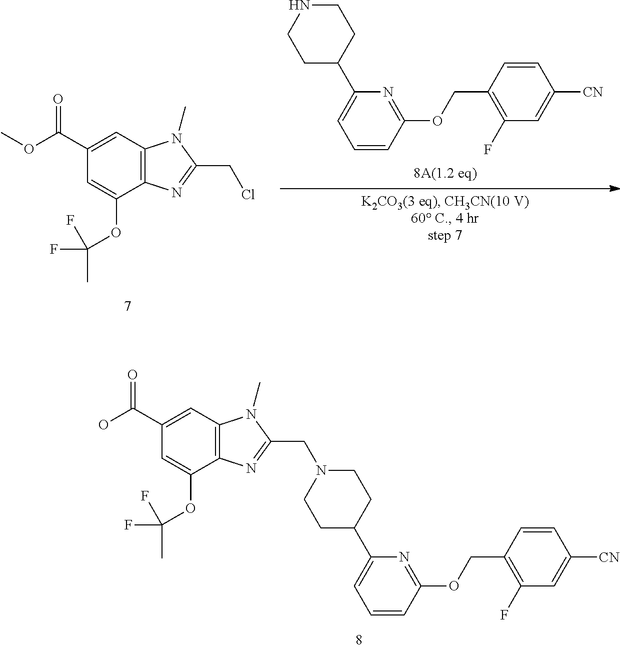 [SMILES: COC(=O)c1cc(OC(C)(F)F)c2nc(CCl)n(C)c2c1 ; N#Cc1ccc(COc2cccc(C3CCNCC3)n2)c(F)c1 ; "8A(1.2 eq)" ; "K2CO3(3 eq), CH3CN(10 V)60° C., 4 hrstep 7" ; Cn1c(CN2CCC(c3cccc(OCc4ccc(C#N)cc4F)n3)CC2)nc2c(OC(C)(F)F)cc(C(=O)O)cc21]

To a solution of 7 (80 mg, 251.02 μmol, 1 eq), 8A (93.79 mg, 301.22 μmol, 1.2 eq), in ACN (0.1 mL) was added K₂CO₃ (34.69 mg, 251.02 μmol, 1 eq). The mixture was stirred at 60° C. for 4 hr. LC-MS showed 7 was consumed completely and the desired mass was detected as a main peak. The mixture was diluted with H₂O (5 mL), extracted with EtOAc (5 mL*3). The combined organic layer was washed with 10 mL brine, dried over Na₂SO₄, filtered and the filtrate was concentrated to give a crude product. The residue was purified by column chromatography (SiO₂, Petroleum ether/Ethyl acetate=5/1). 8 (100 mg, 168.47 μmol, 67% yield) was obtained as a yellow solid.

LCMS: RT=0.445 min, MS cal.: 593.2, 594.2, [M+H]⁺=594.2, 595.2

¹H NMR (400 MHz, CHLOROFORM-d) δ=8.01 (s, 1H), 7.90 (s, 1H), 7.67-7.60 (m, 1H), 7.54 (t, J=7.6 Hz, 1H), 7.45 (d, J=8.0 Hz, 1H), 7.39-7.36 (dd, J=9.2, 1.2 Hz, 1H), 6.76 (d, J=7.2 Hz, 1H), 6.66 (d, J=8.0 Hz, 1H), 5.50 (s, 2H), 4.00 (d, 2H), 3.97 (s, 3H), 3.92 (s, 1H), 2.96-2.94 (m, 1H), 2.65-2.56 (m, 1H), 2.34-2.26 (m, 2H), 2.09 (t, J=7.2 Hz, 3H), 1.95-1.67 (m, 4H), 1.68-1.49 (m, 5H)

### General Procedure for Preparation of Compound 180 (9):

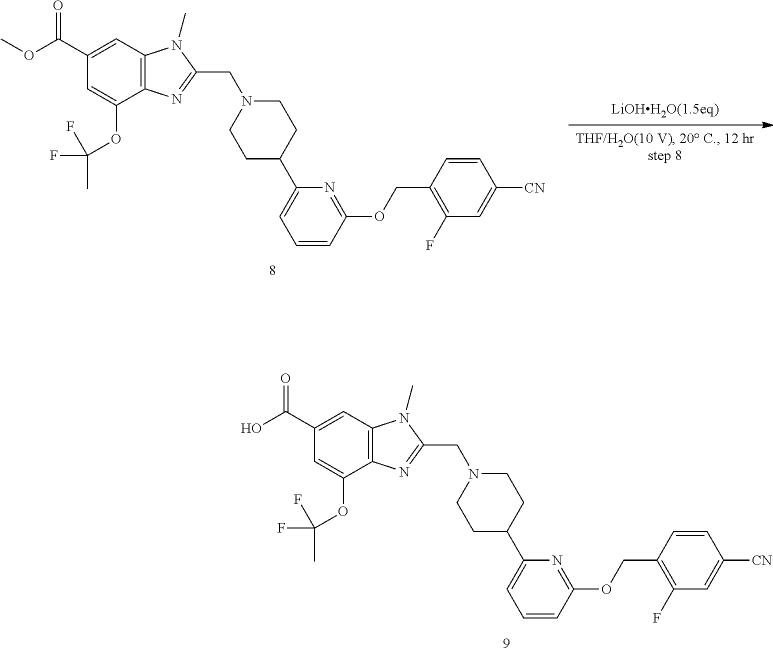 [SMILES: COC(=O)c1cc(OC(C)(F)F)c2nc(CN3CCC(c4cccc(OCc5ccc(C#N)cc5F)n4)CC3)n(C)c2c1 ; "LiOH·H2O(1.5eq)" ; "THF/H2O(10 V), 20° C., 12 hrstep 8" ; Cn1c(CN2CCC(c3cccc(OCc4ccc(C#N)cc4F)n3)CC2)nc2c(OC(C)(F)F)cc(C(=O)O)cc21]

To a solution of 8 (80 mg, 134.77 μmol, 1 eq) in THF (0.7 mL) was added LiOH·H₂O (8.48 mg, 202.16 μmol, 1.5 eq) in H₂O (0.3 mL). The mixture was stirred at 20° C. for 12 hr. LC-MS showed 8 was consumed completely and the desired mass was detected as a main peak. The mixture was purified directly. The residue was purified by prep-HPLC (column: Waters Xbridge BEH C18 100*30 mm*10 um; mobile phase: [H₂O (10 mM NH₄HCO₃)-ACN]; gradient: 10%-40% B over 8.0 min). 2-((4-(6-((4-Cyano-2-fluorobenzyl)oxy)pyridin-2-yl)piperidin-1-yl)methyl)-4-(1,1-difluoroethoxy)-1-methyl-1H-benzo[d]imidazole-6-carboxylic acid (28.19 mg, 48.64 μmol, 36% yield) was obtained as a white solid.

LCMS: RT=2.606 min, MS cal.: 579.2, 580.2, [M+H]⁺=580.2, 581.2

¹H NMR (400 MHz, DMSO-d6) δ=8.05 (s, 1H), 7.88 (d, J=10.0 Hz, 1H), 7.69 (d, J=3.6 Hz, 2H), 7.64 (t, J=7.2 Hz, 2H), 6.87 (d, J=7.2 Hz, 1H), 6.71 (d, J=8.0 Hz, 1H), 5.45 (s, 2H), 3.94 (s, 3H), 3.83 (s, 2H), 2.90 (d, J=10.4 Hz, 2H), 2.61-2.56 (m, 1H), 2.19 (t, J=11.2 Hz, 2H), 2.01 (t, J=14.0 Hz, 3H), 1.79-1.72 (m, 2H), 1.71-1.60 (m, 2H)

### Example A267—Preparation of Compound 174 and Compound 173. rel-(S)-2-((4-(2-(1-(4-cyano-2-fluorophenyl)ethoxy)phenyl)piperidin-1-yl)methyl)-4-(difluoromethoxy)-1-methyl-1H-benzo[d]imidazole-6-carboxylic acid (Compound 174) (8A) & rel-(R)-2-((4-(2-(1-(4-cyano-2-fluorophenyl)ethoxy)phenyl)piperidin-1-yl)methyl)-4-(difluoromethoxy)-1-methyl-1H-benzo[d]imidazole-6-carboxylic acid (Compound 173)

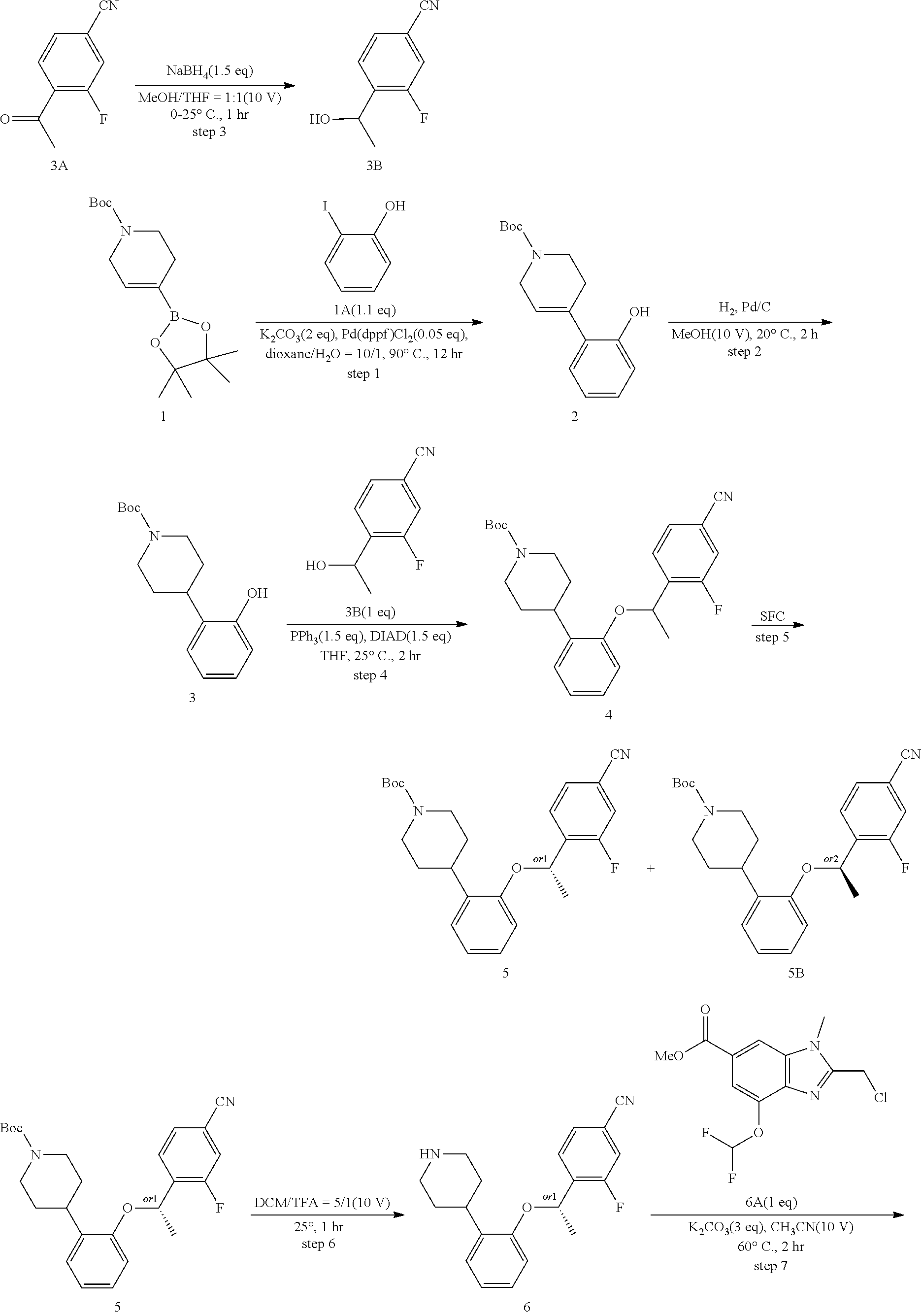

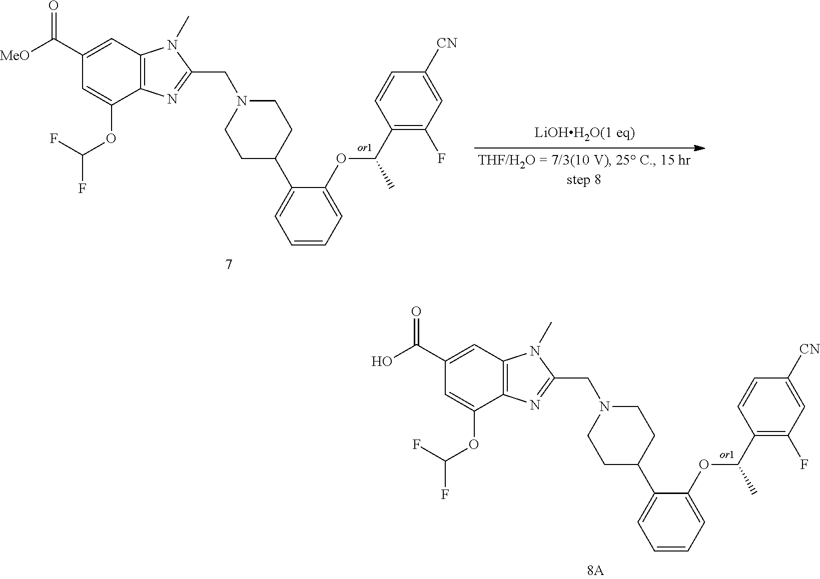 [SMILES: COC(=O)c1cc(OC(F)F)c2nc(CN3CCC(c4ccccc4O[C@@H](C)c4ccc(C#N)cc4F)CC3)n(C)c2c1 ; C[C@H](Oc1ccccc1C1CCN(Cc2nc3c(OC(F)F)cc(C(=O)O)cc3n2C)CC1)c1ccc(C#N)cc1F]

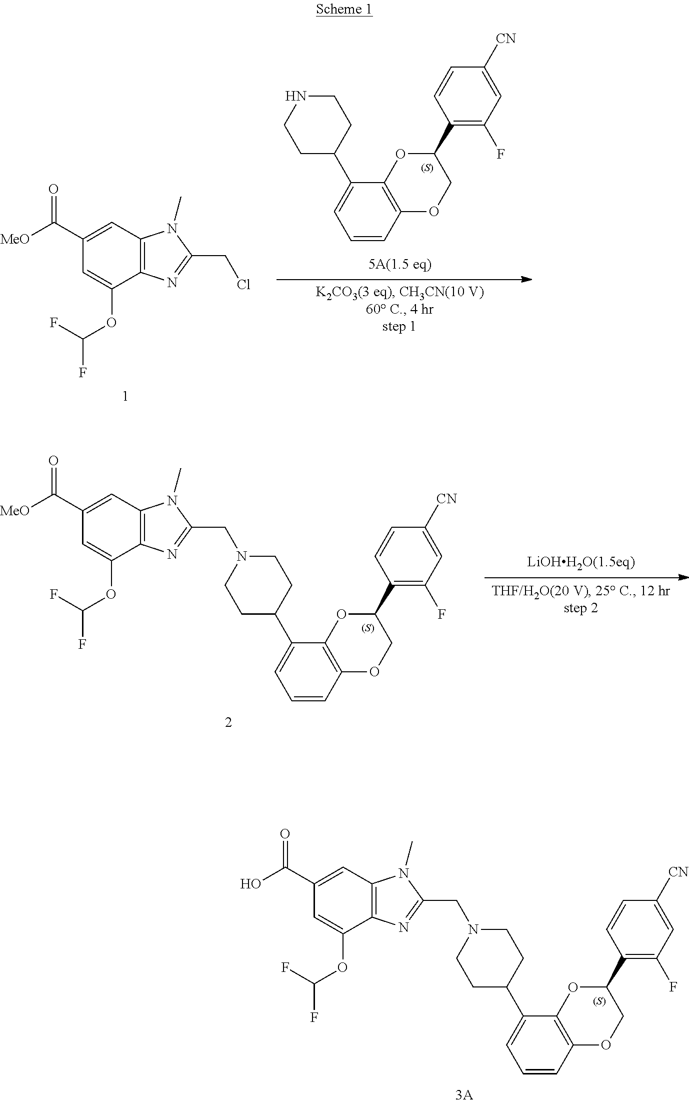

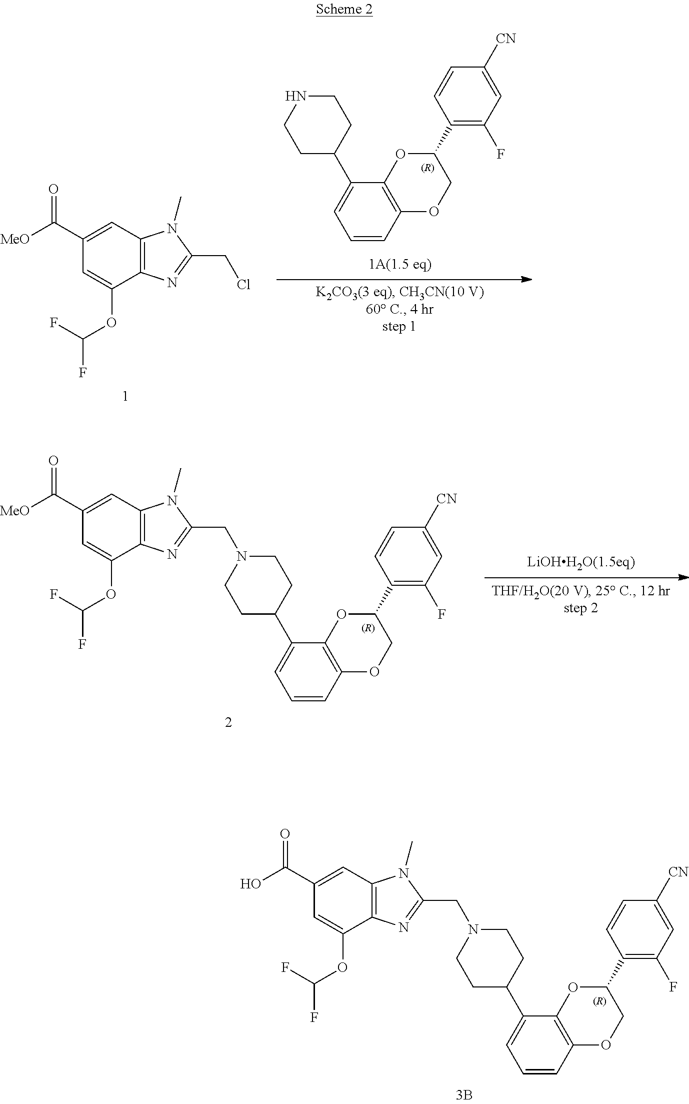

### General Procedure for Preparation of Intermediate 2:5

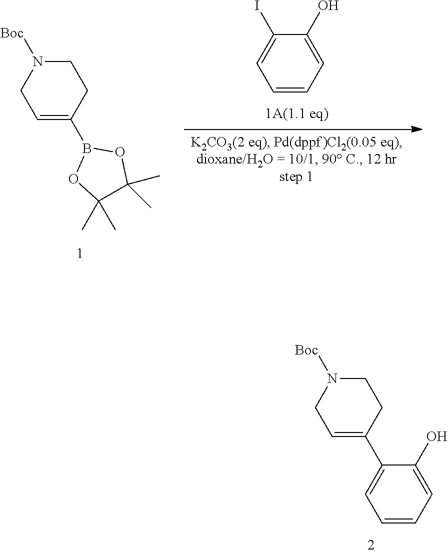

To a mixture of 1 (1.33 g, 4.30 mmol, 1 eq), 1A (1.04 g, 4.73 mmol, 534.64 μL, 1.1 eq) and K₂CO₃ (1.19 g, 8.60 mmol, 2 eq) in dioxane (10 mL) and H₂O (1 mL) was added Pd(dppf)Cl₂ (157.36 mg, 215.07 μmol, 0.05 eq) at 25° C. The mixture was de-gassed under reduced pressure and recharged with N₂. The mixture was stirred at 90° C. for 15 hours. LC-MS showed 1 was consumed completely and one main peak with desired mass was detected. The residue was poured into water (50 mL). The aqueous phase was extracted with ethyl acetate (20 mL*3). The combined organic phase was washed with saturated brine (50 mL*1), dried with anhydrous Na₂SO₄, filtered, and concentrated in vacuum. The residue was purified by column chromatography (SiO₂, PE/EtOAc=I/O to 2/1). 2 (2.8 g, 9.36 mmol, 72.54% yield) was obtained as a colorless oil.

LCMS: RT=0.511 min, MS cal.: 275.2, [M+H−t-Bu]⁺=220.2

HPLC: product: RT=3.028 min, purity: 92.05%

¹H NMR (400 MHz, METHANOL-d4) δ=7.09-7.03 (m, 2H), 6.80-6.74 (m, 2H), 5.77 (br s, 1H), 4.02 (br s, 2H), 3.59 (br t, J=4.8 Hz, 2H), 2.54-2.48 (m, 2H), 1.49 (s, 9H)

### General Procedure for Preparation of Intermediate 3:

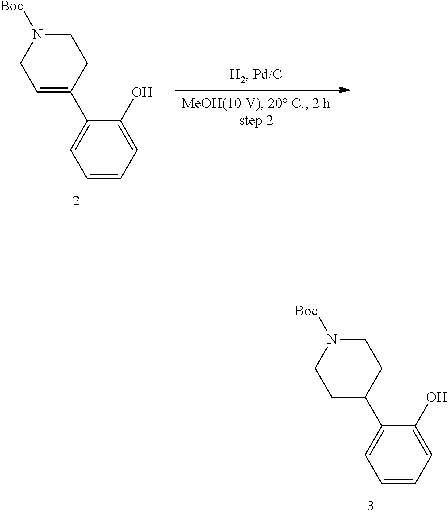 [SMILES: CC(C)(C)OC(=O)N1CC=C(c2ccccc2O)CC1 ; "H2, Pd/C" ; "MeOH(10 V), 20° C., 2 hstep 2" ; CC(C)(C)OC(=O)N1CCC(c2ccccc2O)CC1]

To a solution of 2 (1.4 g, 5.08 mmol, 1 eq) in MeOH (30 mL) was added Pd/C (250.00 mg, 234.92 μmol, 10% purity, 4.62e-2 eq) under N₂ atmosphere. The suspension was degassed and purged with H₂ for 3 times. The mixture was stirred under H₂ (15 Psi) at 25° C. for 1 hr. LC-MS showed 2 was consumed completely and one main peak with desired mass was detected. The mixture was filtered and concentrated in vacuum. The crude product was used into the next step without further purification. 3 (2.7 g, crude) was obtained as a yellow solid.

LCMS: RT=0.527 min, MS cal.: 277.2, [M+H−t-Bu]⁺=222.1

HPLC: product: RT=3.019 min, purity: 85.88%

¹H NMR (400 MHz, METHANOL-d4) δ=7.05 (dd, J=1.6, 7.6 Hz, 1H), 6.99 (dt, J=1.6, 8.0 Hz, 1H), 6.79-6.72 (m, 2H), 4.19 (br d, J=13.2 Hz, 2H), 3.08 (tt, J=3.6, 12.4 Hz, 1H), 2.94-2.73 (m, 2H), 1.79 (br d, J=12.4 Hz, 2H), 1.62-1.51 (m, 2H), 1.47 (s, 9H)

### General Procedure for Preparation of Intermediate 3B:

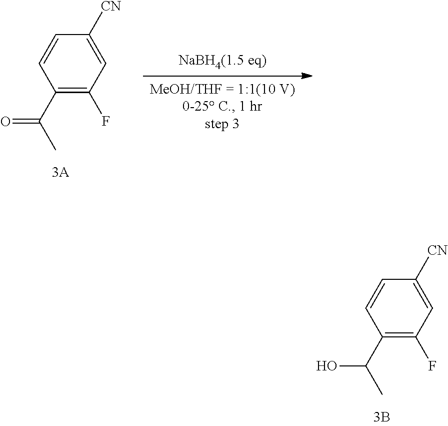

A mixture of 3A (400 mg, 2.45 mmol, 1 eq) in MeOH (4 mL) and THF (4 mL) was added at 25° C., and the mixture was stirred at 0° C. for 10 min. Then NaBH₄ (139.13 mg, 3.68 mmol, 1.5 eq) was added at 0° C. The mixture was stirred at 25° C. for 40 min. TLC (SiO₂, Petroleum ether:Ethyl acetate=3:1, Rf=0.35) showed the reaction was completed. The residue was poured into water (50 mL). The aqueous phase was extracted with ethyl acetate (20 mL*3). The combined organic phase was washed with brine (50 mL*1), dried with anhydrous Na₂SO₄, filtered, and concentrated in vacuum. The residue was purified by column chromatography (SiO₂, Petroleum ether:Ethyl acetate=1/0 to 2/1). 3B (285 mg, 1.68 mmol, 68.69% yield) was obtained as a colorless oil.

HPLC: product: RT=1.895 min, purity: 97.56%

¹H NMR (400 MHz, DMSO-d6) δ=7.79-7.75 (m, 1H), 7.72-7.68 (m, 2H), 5.56 (d, J=4.4 Hz, 1H), 5.04-4.96 (m, 1H), 1.33 (d, J=6.4 Hz, 3H)

### General Procedure for Preparation of Intermediate 4:

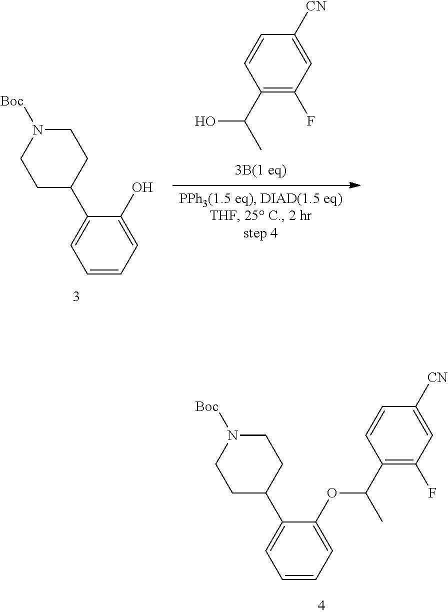

To a solution of 3B (220 mg, 1.33 mmol, 1 eq) in THF (5 mL) was added PPh₃ (524.05 mg, 2.00 mmol, 1.5 eq) and 3 (554.16 mg, 2.00 mmol, 1.5 eq) at 25° C. After addition, the mixture was stirred at this temperature for 30 min, and then DIAD (404.01 mg, 2.00 mmol, 387.36 μL, 1.5 eq) was added. The resulting mixture was stirred at 25° C. for 2 hr. LC-MS showed 3B was consumed completely and one main peak with desired mass was detected. The residue was poured into water (30 mL). The aqueous phase was extracted with ethyl acetate (10 mL*3). The combined organic phase was washed with saturated brine (30 mL*1), dried with anhydrous Na₂SO₄, filtered and concentrated in vacuum. The residue was purified by prep-TLC (SiO₂, Petroleum ether:Ethyl acetate=3:1, Rf=0.55). 4 (410 mg, 70.15% yield) was obtained as a yellow oil.

LCMS: RT=0.665 min, MS cal.: 424.2, [M+Na]⁺=447.2

HPLC: product: RT=3.911 min, purity: 98.15%

¹H NMR (400 MHz, DMSO-d6) δ=7.90 (dd, J=1.2, 10.4 Hz, 1H), 7.74-7.69 (m, 1H), 7.67-7.61 (m, 1H), 7.17 (dd, J=1.2, 7.6 Hz, 1H), 7.10-7.03 (m, 1H), 6.90-6.84 (m, 1H), 6.77 (d, J=8.4 Hz, 1H), 5.75-5.69 (m, 1H), 4.14-4.03 (m, 2H), 3.17-3.08 (m, 1H), 2.93-2.75 (m, 2H), 1.76 (br d, J=12.4 Hz, 1H), 1.66 (br d, J=12.8 Hz, 1H), 1.60 (d, J=6.4 Hz, 3H), 1.55-1.44 (m, 2H), 1.42 (s, 9H)

### General Procedure for Preparation of Intermediate 5 & 5B:

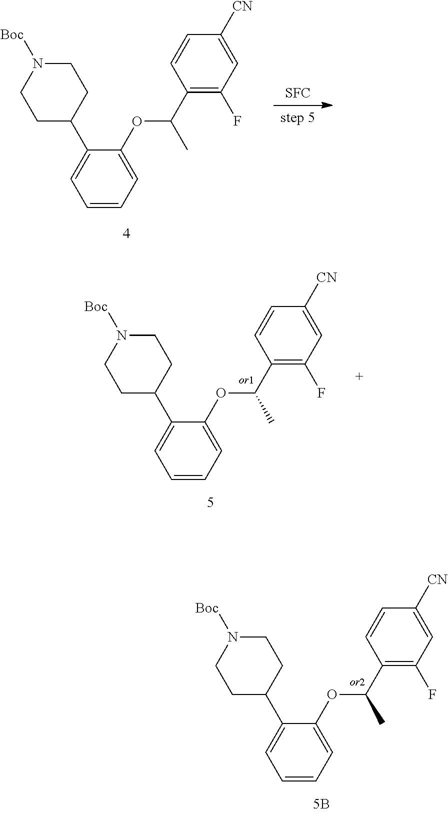

The residue was separated by SFC (column: CHIRALPAK AD-3, 100×4.6 mm, I.D., 3 um; mobile phase: A: Hexane B: EtOH (0.1% IPAm, v/v)). 5 (190 mg, 439.48 μmol, 32.99% yield) and 5B (202 mg, 457.53 μmol, 34.35% yield) was obtained as a yellow solid.

HPLC: 5: RT=3.929 min, purity: 98.19%

HPLC: 5B: RT=3.926 min, purity: 96.15%

¹H NMR (400 MHz, DMSO-d6) δ=7.90 (dd, J=1.6, 10.4 Hz, 1H), 7.72 (dd, J=1.4, 8.0 Hz, 1H), 7.66-7.61 (m, 1H), 7.17 (dd, J=1.6, 7.6 Hz, 1H), 7.09-7.04 (m, 1H), 6.90-6.85 (m, 1H), 6.77 (d, J=8.0 Hz, 1H), 5.72 (q, J=6.4 Hz, 1H), 4.09 (br d, J=6.0 Hz, 2H), 3.18-3.08 (m, 1H), 2.83 (br d, J=2.8 Hz, 2H), 1.76 (br d, J=12.4 Hz, 1H), 1.66 (br d, J=12.4 Hz, 1H), 1.61 (d, J=6.4 Hz, 3H), 1.50 (ddd, J=4.4, 7.6, 12.4 Hz, 2H), 1.42 (s, 9H)

1H NMR (400 MHz, DMSO-d6) δ=7.90 (dd, J=1.2, 10.4 Hz, 1H), 7.75-7.70 (m, 1H), 7.67-7.61 (m, 1H), 7.17 (dd, J=1.6, 7.6 Hz, 1H), 7.09-7.03 (m, 1H), 6.90-6.85 (m, 1H), 6.77 (d, J=8.0 Hz, 1H), 5.72 (q, J=6.4 Hz, 1H), 4.09 (br d, J=6.4 Hz, 2H), 3.18-3.07 (m, 1H), 2.84 (br s, 2H), 1.76 (br d, J=12.6 Hz, 1H), 1.66 (br d, J=12.8 Hz, 1H), 1.61 (d, J=6.4 Hz, 3H), 1.50 (ddd, J=4.4, 7.6, 12.4 Hz, 2H), 1.42 (s, 9H)

### General Procedure for Preparation of Intermediate 6:

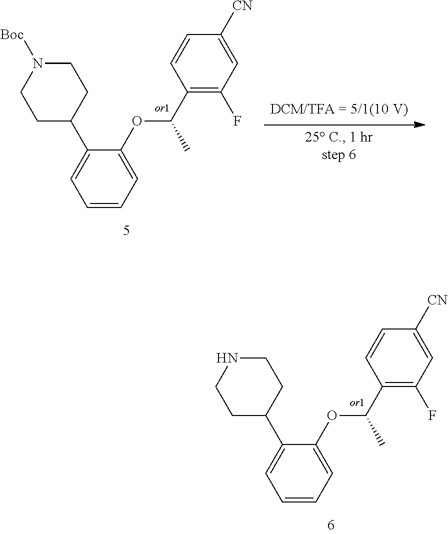

To a solution of 5 (140 mg, 329.79 μmol, 1 eq) in DCM (1.5 mL) was added TFA (460.50 mg, 4.04 mmol, 300.00 μL, 12.25 eq). The mixture was stirred at 25° C. for 1 hr. LC-MS showed 5 was consumed completely and one main peak with desired mass was detected. The mixture was filtered and concentrated in vacuum. The crude product was used into the next step without further purification. 6 (120 mg, crude) was obtained as a yellow solid.

LCMS: RT=0.396 min, MS cal.: 324.2, [M+H]⁺=325.2

HPLC: product: RT=2.338 min, purity: 97.58%

¹H NMR (400 MHz, METHANOL-d4) δ=7.65-7.61 (m, 1H), 7.61-7.58 (m, 1H), 7.57-7.53 (m, 1H), 7.20 (dd, J=1.6, 7.6 Hz, 1H), 7.09-7.03 (m, 1H), 6.93-6.88 (m, 1H), 6.72 (d, J=8.0 Hz, 1H), 5.74 (q, J=6.4 Hz, 1H), 3.37 (br s, 1H), 3.33 (br d, J=3.2 Hz, 2H), 3.00 (tt, J=2.8, 12.2 Hz, 2H), 2.04-1.97 (m, 1H), 1.96-1.89 (m, 1H), 1.88-1.74 (m, 2H), 1.69 (d, J=6.4 Hz, 3H)

### General Procedure for Preparation of Intermediate 7:

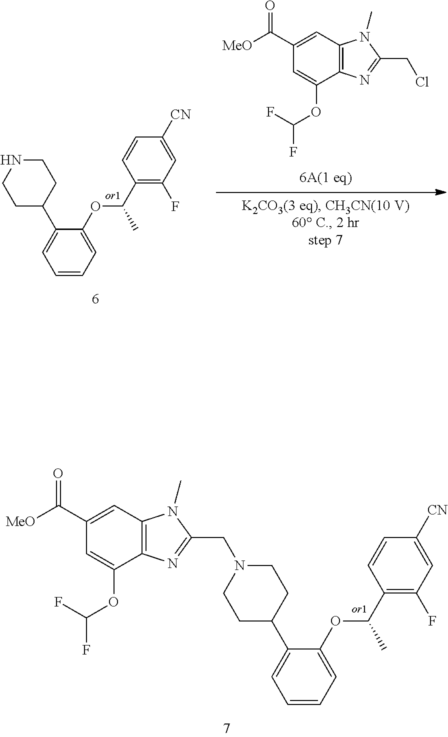 [SMILES: C[C@H](Oc1ccccc1C1CCNCC1)c1ccc(C#N)cc1F ; COC(=O)c1cc(OC(F)F)c2nc(CCl)n(C)c2c1 ; "6A(1 eq)" ; "K2CO3(3 eq), CH3CN(10 V)60° C., 2 hrstep 7" ; COC(=O)c1cc(OC(F)F)c2nc(CN3CCC(c4ccccc4O[C@@H](C)c4ccc(C#N)cc4F)CC3)n(C)c2c1]

To a solution of 6A (70 mg, 229.75 μmol, 1 eq) in ACN (1 mL) was added K₂CO₃ (95.26 mg, 689.26 μmol, 3 eq) and 6 (96.89 mg, 298.68 μmol, 1.3 eq). The mixture was de-gassed under reduced pressure and recharged with N₂. The mixture was stirred at 60° C. for 1 hr. LC-MS showed 6A was consumed completely and one main peak with desired mass was detected. The mixture was filtered and concentrated in vacuum. The residue was purified by prep-TLC (SiO₂, Petroleum ether:Ethyl acetate=2:1, Rf=0.40). 7 (128 mg, 207.48 μmol, 90.31% yield) was obtained as a yellow solid.

LCMS: RT=0.466 min, MS cal.: 592.2, [M+H]⁺=593.3

HPLC: product: RT=2.848 min, purity: 96.06%

¹H NMR (400 MHz, METHANOL-d4) δ=8.12 (d, J=1.2 Hz, 1H), 7.67 (s, 1H), 7.64-7.53 (m, 3H), 7.26 (t, J=74 Hz, 1H), 7.18 (dd, J=1.2, 7.6 Hz, 1H), 7.04-6.98 (m, 1H), 6.89-6.84 (m, 1H), 6.68 (d, J=8.0 Hz, 1H), 5.70 (q, J=6.4 Hz, 1H), 4.03 (s, 3H), 3.96 (s, 3H), 3.93 (s, 2H), 3.16-3.09 (m, 1H), 3.05 (d, J=11.2 Hz, 2H), 2.41-2.32 (m, 2H), 1.90-1.74 (m, 4H), 1.66 (d, J=6.3 Hz, 3H)

### General Procedure for Preparation of Compound 174 (8A):

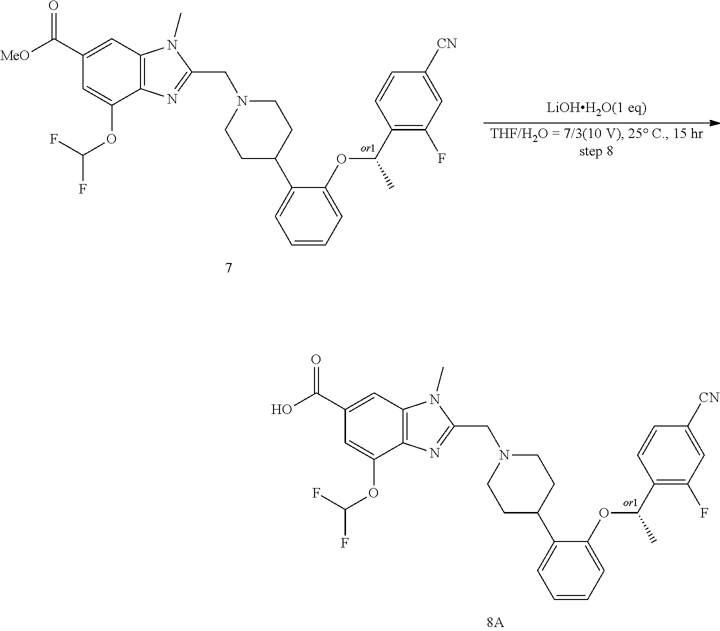

To a solution of methyl 7 (100 mg, 168.75 μmol, 1 eq) in THF (1.4 mL) and H₂O (0.6 mL) was added LiOH·H₂O (7.08 mg, 168.75 μmol, 1 eq). The mixture was stirred at 25° C. for 15 hr. LC-MS showed 7 was consumed completely and one main peak with desired mass was detected.

The mixture was filtered and concentrated in vacuum. The residue was purified by prep-HPLC (column: Waters Xbridge Prep OBD C18 150*40 mm*10 um; mobile phase: [H₂O (10 mM NH₄HCO₃)-ACN]; gradient: 15%-65% B over 8.0 min). rel-(S)-2-((4-(2-(1-(4-Cyano-2-fluorophenyl)ethoxy)phenyl)piperidin-1-yl)methyl)-4-(difluoromethoxy)-1-methyl-1H-benzo[d]imidazole-6-carboxylic acid (28.35 mg, 48.78 μmol, 28.91% yield) was obtained as a white solid.

LCMS: RT=2.707 min, MS cal.: 578.2, [M+H]⁺=579.3.

HPLC: RT=11.374 min, purity: 99.55%

¹H NMR (400 MHz, METHANOL-d4) δ=8.11 (d, J=1.2 Hz, 1H), 7.69 (s, 1H), 7.65-7.60 (m, 1H), 7.60-7.57 (m, 1H), 7.57-7.54 (m, 1H), 7.25 (t, J=74.4 Hz, 1H), 7.20 (dd, J=1.6, 7.6 Hz, 1H), 7.02 (dt, J=1.6, 8.4 Hz, 1H), 6.87 (dt, J=0.8, 7.6 Hz, 1H), 6.69 (d, J=8.0 Hz, 1H), 5.71 (q, J=6.8 Hz, 1H), 4.03 (s, 3H), 4.00 (s, 2H), 3.18-3.10 (m, 3H), 2.51-2.41 (m, 2H), 1.93-1.77 (m, 4H), 1.67 (d, J=6.4 Hz, 3H)

### General Procedure for Preparation of Intermediate 6B:

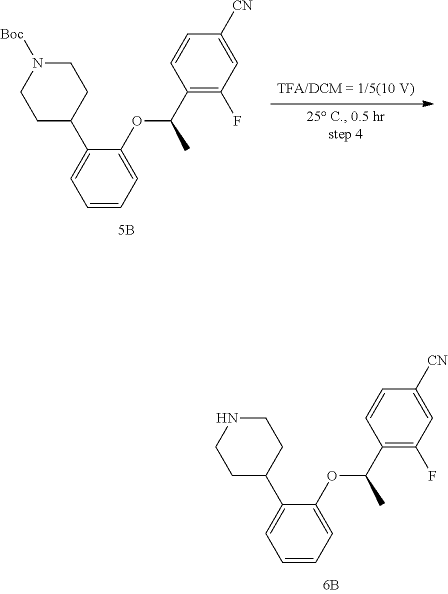

A mixture of 5B (185 mg, 435.80 μmol, 1 eq) in TFA (0.3 mL) and DCM (1.5 mL) was stirred at 25° C. for 0.5 h. The color changes to yellow. LCMS showed the reaction was completed. The reaction mixture was quenched by addition Sat. NaHCO₃10 mL at 25° C., and then diluted with EA 5 mL and extracted with EA 30 mL (10 mL*3). The combined organic layers were washed with brine (30 mL), dried over Na₂SO₄, filtered, and concentrated under reduced pressure to give a residue. The mixture was used for the next step directly without purification. 6B (133 mg, 400.36 μmol, 91.87% yield) was obtained as colorless oil.

LCMS: RT=0.402 min, MS cal.: 324.2/325.2, [M+H]⁺=325.1/326.1

HPLC: RT=1.952 min, purity: 97.65%

¹H NMR (ET83165-50-P1A, 400 MHz, METHANOL-d4) δ=7.66-7.53 (m, 3H), 7.20 (dd, J=1.2, 7.6 Hz, 1H), 7.05 (dt, J=2.0, 8.4 Hz, 1H), 6.94-6.87 (m, 1H), 6.72 (d, J=8.0 Hz, 1H), 5.73 (q, J=6.0 Hz, 1H), 2.94 (tt, J=2.8, 12.8 Hz, 2H), 1.97 (br d, J=13.2 Hz, 1H), 1.92-1.85 (m, 1H), 1.84-1.73 (m, 2H), 1.68 (d, J=6.0 Hz, 3H)

### General Procedure for Preparation of Intermediate 3:

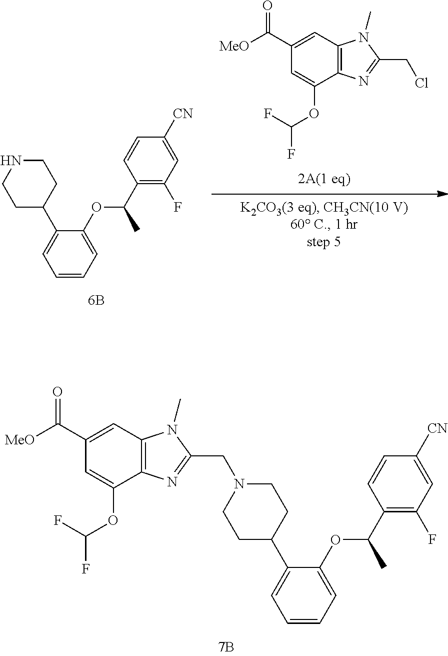

A mixture of 6B (70 mg, 229.75 μmol, 1 eq), 2A (96.89 mg, 298.68 μmol, 1.3 eq) and K₂CO₃ (95.26 mg, 689.26 μmol, 3 eq) in ACN (1 mL) was degassed and purged with N₂ for 3 times. The mixture was stirred 60° C. for 2 hours. The color changes to yellow. TLC (SiO₂, PE:EA=2:1, Rf=0.60) showed the reaction was completed. The mixture was filtered and concentrated in vacuum. The residue was purified by prep-TLC (SiO₂, PE:EA=2:1, Rf=0.55). 7B (127 mg, 211.22 μmol, 91.93% yield) was obtained as a colorless solid.

HPLC: RT=3.315 min, purity: 98.56%

¹H NMR (MHZ, METHANOL-d4) δ=8.13 (d, J=1.2 Hz, 1H), 7.68 (s, 1H), 7.65-7.57 (m, 2H), 7.57-7.53 (m, 1H), 7.27 (t, J=74 Hz, 1H), 7.19 (dd, J=1.6, 7.6 Hz, 1H), 7.05-6.98 (m, 1H), 6.90-6.83 (m, 1H), 6.69 (d, J=8.0 Hz, 1H), 5.70 (q, J=6.4 Hz, 1H), 4.04 (s, 3H), 3.96 (s, 3H), 3.94 (s, 2H), 3.16-3.08 (m, 1H), 3.05 (br d, J=11.2 Hz, 2H), 2.43-2.33 (m, 2H), 1.92-1.75 (m, 4H), 1.67 (d, J=6.4 Hz, 3H)

### General Procedure for Preparation of Compound 173 (8B):

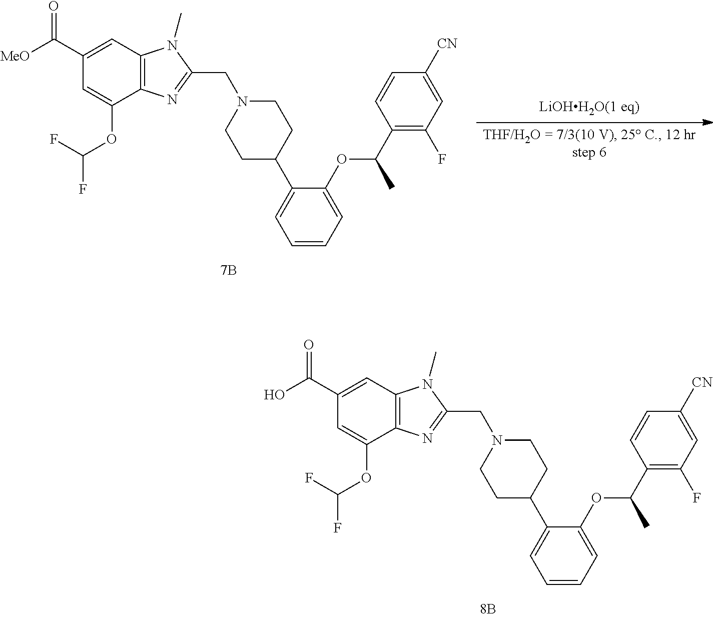

A mixture of 7B (117 mg, 197.43 μmol, 1 eq) and LiOH·H₂O (8.28 mg, 197.43 μmol, 1 eq) in THF (2.8 mL) and H₂O (1.2 mL) was stirred at 25° C. for 12 hours. The color changes to yellow. LCMS showed 89% of desired product was detected. The mixture was concentrated in vacuum. The residue was purified by prep-HPLC (column: Waters Xbridge Prep OBD C18 150*40 mm*10 um; mobile phase: [H₂O (10 mM NH₄HCO₃)-ACN]; gradient: 20%-70% B over 8.0 min). rel-(R)-2-((4-(2-(1-(4-Cyano-2-fluorophenyl)ethoxy)phenyl)piperidin-1-yl)methyl)-4-(difluoromethoxy)-1-methyl-1H-benzo[d]imidazole-6-carboxylic acid (28.75 mg, 46.65 μmol, 23.63% yield) was obtained as a white solid.

LCMS: RT=2.707 min, MS cal.: 578.2/579.2, [M+H]⁺=579.2/580.2.

HPLC: RT=11.382 min, purity: 93.89%

¹H NMR (400 MHz, METHANOL-d4) δ=8.13-8.08 (m, 1H), 7.69 (s, 1H), 7.65-7.57 (m, 2H), 7.57-7.52 (m, 1H), 7.25 (J=74.4 Hz, 1H), 7.20 (d, J=7.6 Hz, 1H), 7.05-6.99 (m, 1H), 6.91-6.84 (m, 1H), 6.69 (d, J=8.0 Hz, 1H), 5.71 (q, J=6.0 Hz, 1H), 4.03 (s, 3H), 4.01 (s, 2H), 3.20-3.07 (m, 3H), 2.54-2.40 (m, 2H), 1.95-1.78 (m, 4H), 1.67 (d, J=6.4 Hz, 3H)

### Example A268—Preparation of Compound 167. 2-((4-(5-((4-Chloro-2-fluorobenzyl)oxy)-2-fluorophenyl)piperidin-1-yl)methyl)-4-(difluoromethoxy)-1-methyl-1H-benzo[d]imidazole-6-carboxylic acid (Compound 167) (5)

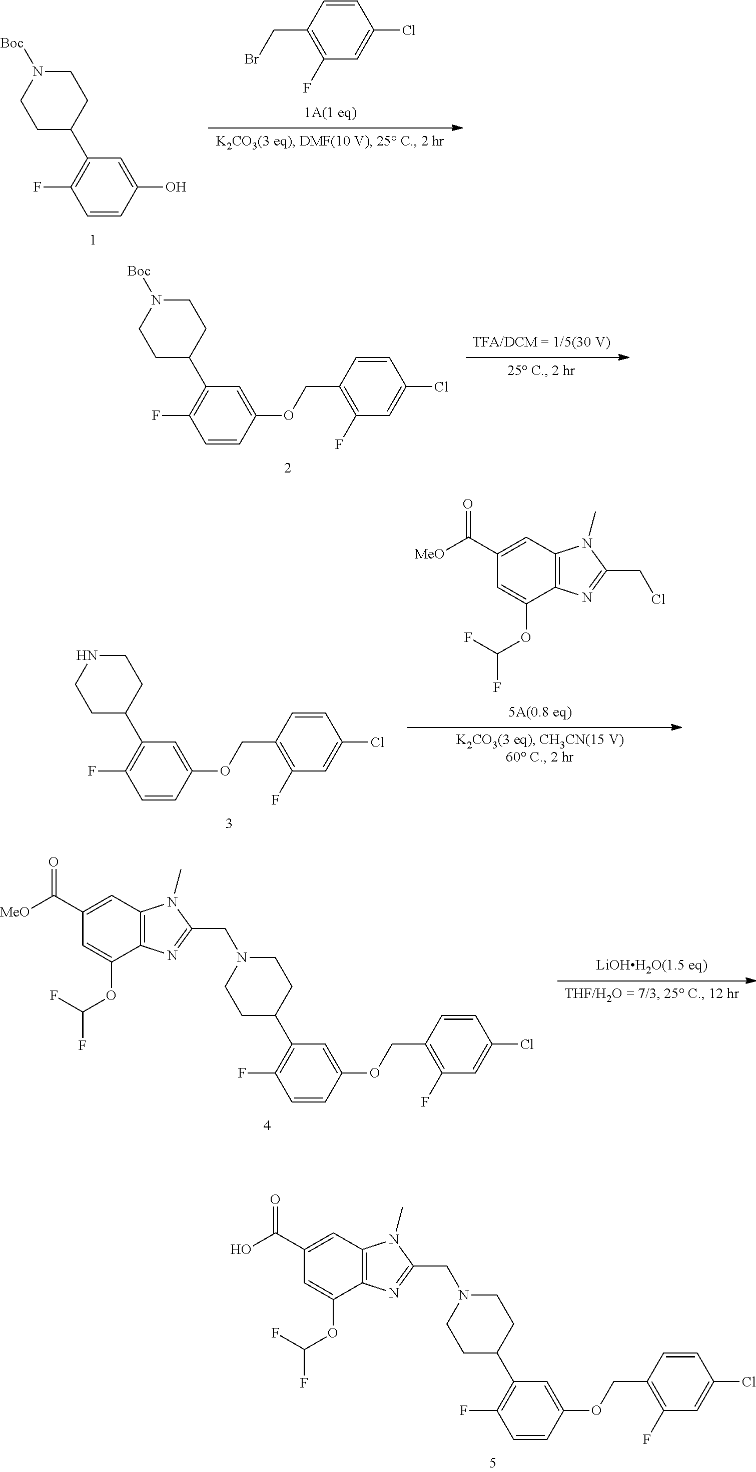

### General Procedure for Preparation of Intermediate 2:

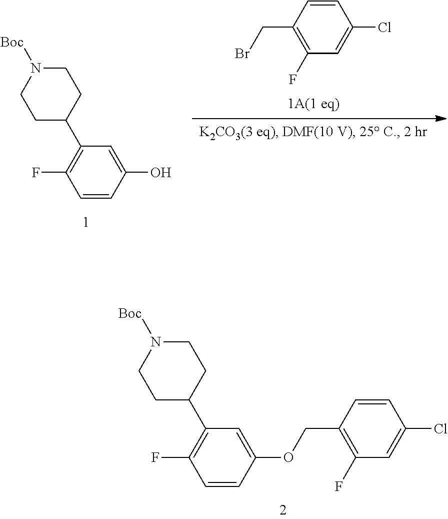

A mixture of 1 (200 mg, 677.17 μmol, 1 eq), 1A (151.33 mg, 677.17 μmol, 1 eq), K₂CO₃ (280.76 mg, 2.03 mmol, 3 eq) in DMF (2 mL) was degassed and purged with N₂ for 3 times, and then the mixture was stirred at 25° C. for 2 hr under N₂ atmosphere. TLC (Petroleum ether:Ethyl acetate=10:1, R_{f}=0.59) indicated 1 was consumed completely and one new spot formed. The reaction was clean according to TLC. The reaction mixture diluted with H₂O 20 mL and extracted with EtOAc 45 mL (15 mL*3). The combined organic layers were washed with brine 15 mL, dried over Na₂SO₄, filtered, and concentrated under reduced pressure to give a residue. The residue was purified by column chromatography (SiO₂, Petroleum ether/Ethyl acetate=10/1 to 1/1). 2 (150 mg, 342.54 μmol, 50.58% yield) was obtained as a colourless oil.

LCMS: RT=1.747 min, MS cal.: 437.2/439.2, [M−55]⁺=382.0/383.9

¹H NMR (400 MHz, CDCl₃) δ=7.44 (t, J=8.0 Hz, 1H), 7.20-7.11 (m, 2H), 6.95 (t, J=9.6 Hz, 1H), 6.85-6.70 (m, 2H), 5.04 (s, 2H), 4.25 (br d, J=13.2 Hz, 2H), 3.06-2.93 (m, 1H), 2.82 (dt, J=1.6, 12.8 Hz, 2H), 1.80 (br d, J=12.8 Hz, 2H), 1.69-1.55 (m, 3H), 1.49 (s, 9H)

### General Procedure for Preparation of Intermediate 3:

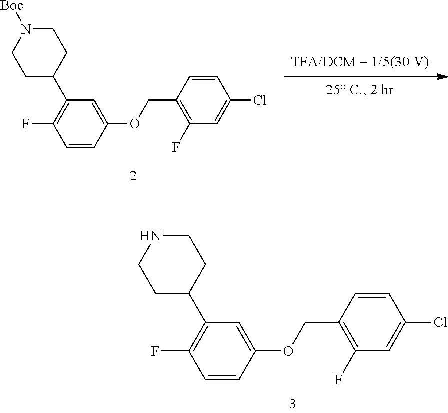

A mixture of 2 (130 mg, 296.87 μmol, 1 eq), TFA (307.00 mg, 2.69 mmol, 0.2 mL, 9.07 eq) in DCM (1 mL) was degassed and purged with N₂ for 3 times, and then the mixture was stirred at 25° C. for 2 hr under N₂ atmosphere. LC-MS showed 2 was consumed completely and one main peak with desired mass was detected. The reaction mixture was concentrated under reduced pressure to remove solvent to give a product without further workup and purification. 3 (130 mg, 287.73 μmol, 96.92% yield, TFA) was obtained as a white solid.

LCMS: RT=1.188 min, MS cal: 337.1/339.1, [M+H]⁺=337.9/339.9

¹H NMR (400 MHz, CDCl₃) δ=7.52-7.39 (m, 1H), 7.23-7.07 (m, 2H), 6.99 (t, J=9.2 Hz, 1H), 6.89-6.75 (m, 2H), 5.05 (s, 2H), 3.65 (br d, J=10.4 Hz, 2H), 3.14 (br s, 3H), 2.09 (br s, 4H)

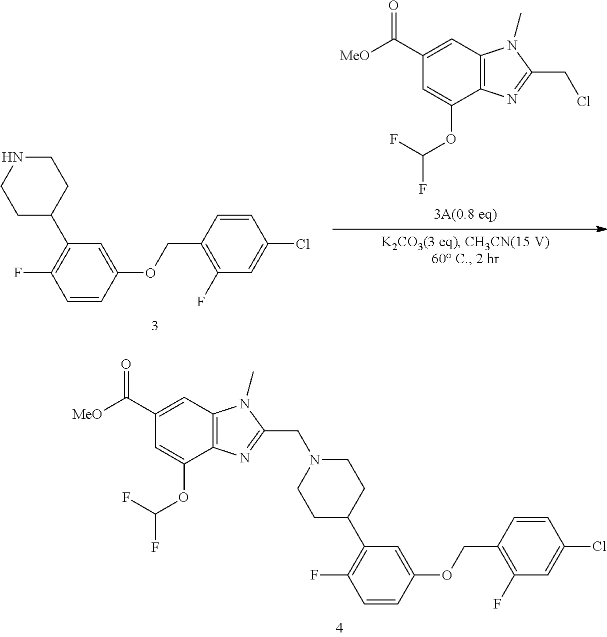 [SMILES: Fc1cc(Cl)ccc1COc1ccc(F)c(C2CCNCC2)c1 ; COC(=O)c1cc(OC(F)F)c2nc(CCl)n(C)c2c1 ; "3A(0.8 eq)" ; "K2CO3(3 eq), CH3CN(15 V)60° C., 2 hr" ; COC(=O)c1cc(OC(F)F)c2nc(CN3CCC(c4cc(OCc5ccc(Cl)cc5F)ccc4F)CC3)n(C)c2c1]

A mixture of 3 (130 mg, 287.73 μmol, 1 eq, TFA), 3A (70.13 mg, 230.18 μmol, 0.8 eq), K₂CO₃ (119.30 mg, 863.19 μmol, 3 eq) in CH₃CN (2 mL) was degassed and purged with N₂ for 3 times, and then the mixture was stirred at 60° C. for 2 hr under N₂ atmosphere. LC-MS showed 3 was consumed completely and one main peak with desired mass was detected. The reaction mixture was diluted with H₂O 20 mL and extracted with EtOAc 45 mL (15 mL*3). The combined organic layers were washed with brine 15 mL, dried over Na₂SO₄, filtered, and concentrated under reduced pressure to give a residue. The residue was purified by column chromatography (SiO₂, Petroleum ether/Ethyl acetate=10/1 to 1/1). 4 (86 mg, 141.91 μmol, 49.32% yield) was obtained as a colourless oil.

LCMS: RT=1.715 min, MS cal.: 605.2/606.2, [M+H]⁺=606.0/607.0

¹H NMR (400 MHz, MeOD) δ=8.13 (d, J=1.2 Hz, 1H), 7.67 (s, 1H), 7.49 (t, J=8.4 Hz, 1H), 7.26 (t, J=74 Hz, 1H), 7.25-7.20 (m, 2H), 6.99-6.92 (m, 1H), 6.87 (dd, J=3.6, 6.0 Hz, 1H), 6.84-6.78 (m, 1H), 5.06 (s, 2H), 4.03 (s, 3H), 3.96 (s, 3H), 3.91 (s, 2H), 3.02 (br d, J=11.2 Hz, 2H), 2.93-2.79 (m, 1H), 2.33 (br dd, J=11.2, 14.0 Hz, 2H), 1.86-1.73 (m, 4H)

### General Procedure for Preparation of Compound 167 (5):

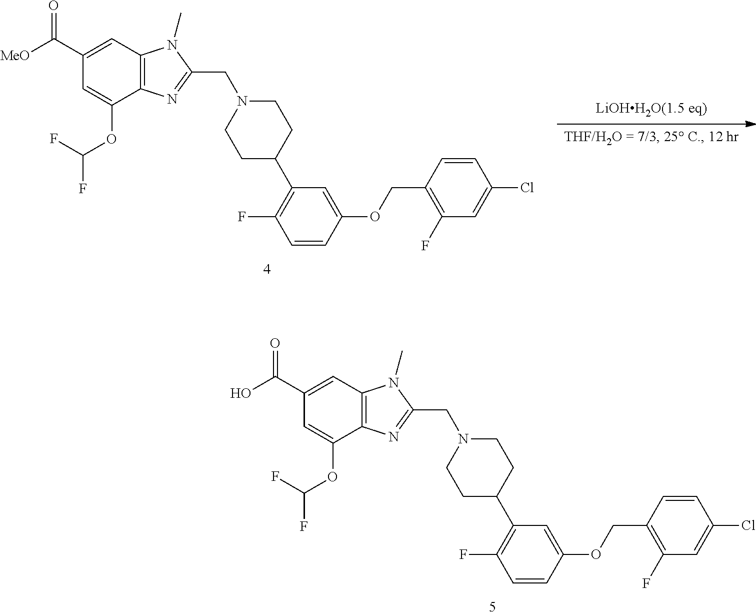

A mixture of 4 (70 mg, 115.51 μmol, 1 eq) in THF (0.7 mL) was added LiOH·H₂O (7.27 mg, 173.27 μmol, 1.5 eq) in H₂O (0.3 mL) was degassed and purged with N₂ for 3 times, and then the mixture was stirred at 25° C. for 12 hr under N₂ atmosphere. LC-MS product RT=1.183 min) showed 4 was consumed completely and one main peak with desired mass was detected. The reaction was purified directly. The residue was purified by prep-HPLC (column: Waters Xbridge Prep OBD C18 150*40 mm*10 um; mobile phase: [H₂O (10 mM NH₄HCO₃)-ACN]; gradient: 15%-65% B over 8.0 min). 2-((4-(5-((4-Chloro-2-fluorobenzyl)oxy)-2-fluorophenyl)piperidin-1-yl)methyl)-4-(difluoromethoxy)-1-methyl-1H-benzo[d]imidazole-6-carboxylic acid (29.36 mg, 49.60 μmol, 42.94% yield) was obtained as a white solid.

LCMS: RT=2.526 min, MS cal.: 591.2/593.2, [M+H]⁺=592.2/594.2

HPLC: RT=12.54 min, purity: 99.34%

¹H NMR (400 MHz, MeOD) δ=8.10 (s, 1H), 7.68 (s, 1H), 7.50 (t, J=8.0 Hz, 1H), 7.27-7.18 (m, 2H), 7.24 (t, J=74.4 Hz, 1H), 7.00-6.92 (m, 1H), 6.88 (dd, J=3.2, 5.6 Hz, 1H), 6.81 (td, J=3.6, 8.4 Hz, 1H), 5.06 (s, 2H), 4.02 (s, 3H), 3.95 (s, 2H), 3.07 (br d, J=11.2 Hz, 2H), 2.93-2.83 (m, 1H), 2.43-2.32 (m, 2H), 1.85-1.76 (m, 4H)

### Example A269—Preparation of Compound 138. 2-((4-(3-Chloro-2-((4-cyano-2-fluorobenzyl)oxy)phenyl)piperidin-1-yl)methyl)-4-(difluoromethoxy)-1-methyl-1H-benzo[d]imidazole-6-carboxylic acid (Compound 138) (7)

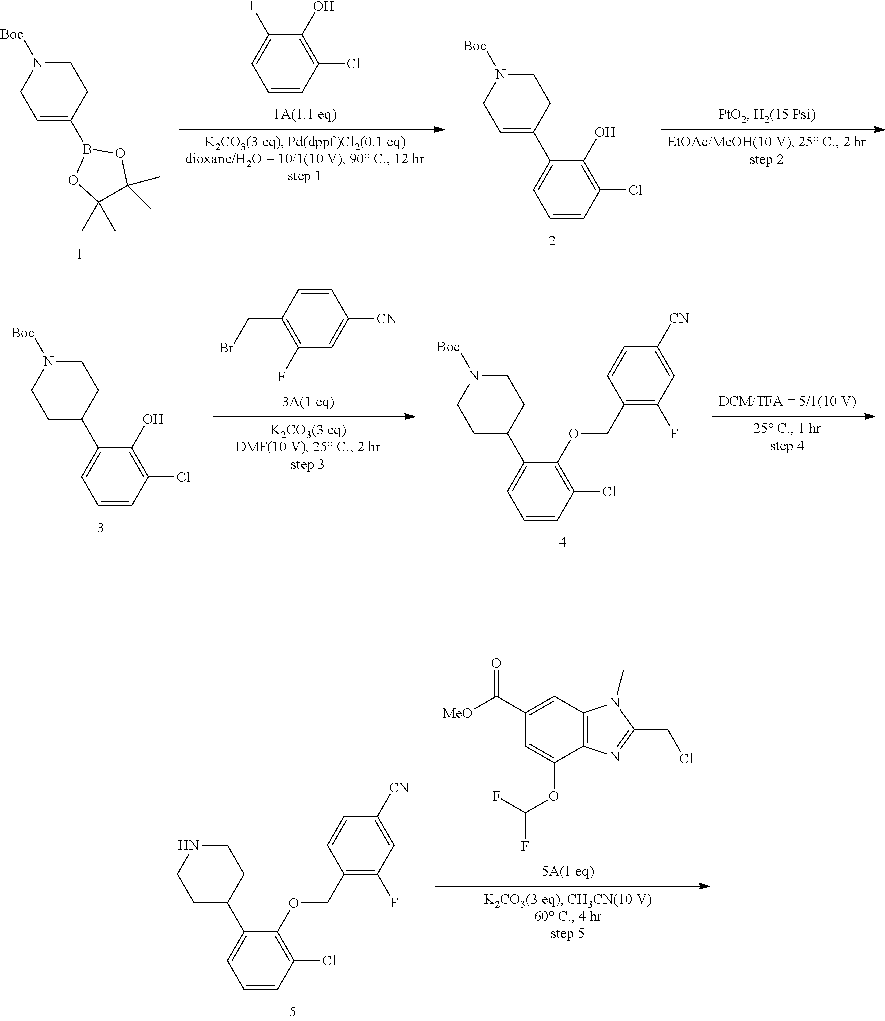

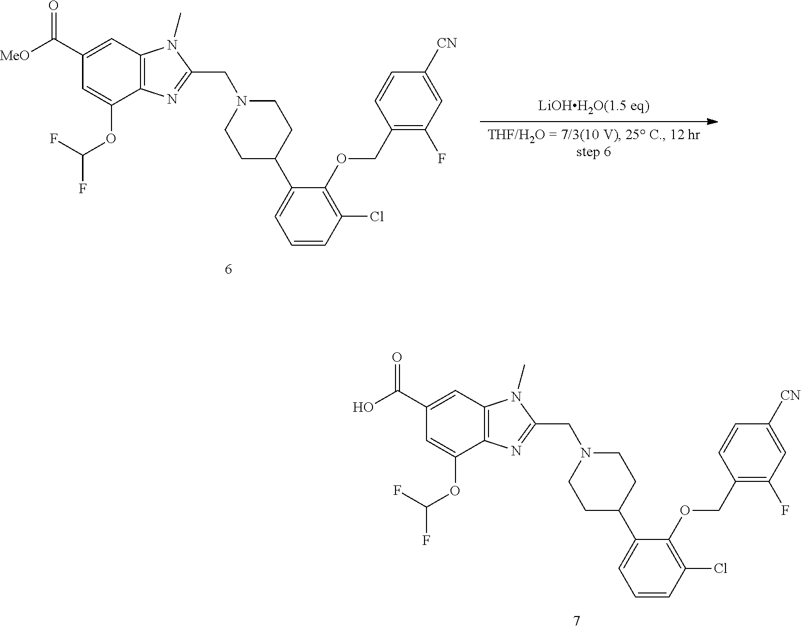

### General Procedure for Preparation of Intermediate 2:

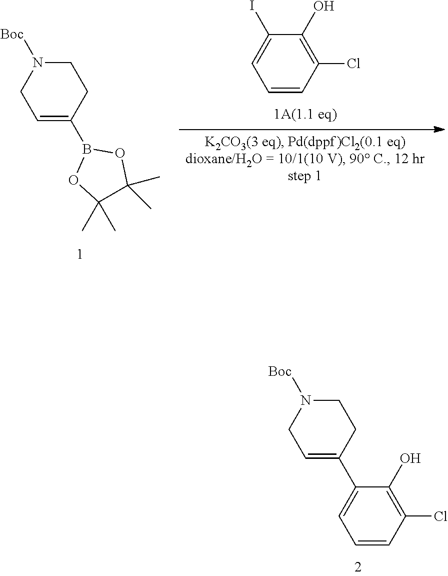

To a solution of 1 (1 g, 3.23 mmol, 1 eq) in dioxane (10 mL) was added 1A (905.21 mg, 3.56 mmol, 1.1 eq), K₂CO₃ (1.34 g, 9.70 mmol, 3 eq) in H₂O (1 mL) and Pd(dppf)Cl₂ (236.64 mg, 323.41 μmol, 0.1 eq). The mixture was stirred at 90° C. for 12 hr. TLC (Petroleum ether/Ethyl acetate=10/1, product R_{f}=0.20) indicated 1 was consumed completely and one new spot formed. The reaction was clean according to TLC. The reaction mixture was filtered through silica gel and the organic phase was added H₂O 20 mL and extracted with EtOAc 60 mL (20 mL*3). The combined organic layers were washed with brine 20 mL, dried over Na₂SO₄, filtered, and concentrated under reduced pressure to give a residue. The residue was purified by column chromatography (SiO₂, Petroleum ether/Ethyl acetate=50/1 to 15/1). 2 (790 mg, 2.55 mmol, 79% yield) was obtained as a yellow solid.

LCMS: RT=0.541 min, MS cal.: 309.1, 311.1, [M−55]⁺=254.1, 256.1

¹H NMR (400 MHz, CHLOROFORM-d) δ=7.25 (dd, J=1.6, 7.6 Hz, 1H), 7.06 (dd, J=1.2, 8.0 Hz, 1H), 6.88-6.81 (m, 1H), 5.88 (s, 1H), 5.76 (s, 1H), 4.08 (d, J=2.8 Hz, 2H), 3.63 (t, J=5.6 Hz, 2H), 2.51 (d, J=1.6 Hz, 2H), 1.50 (s, 9H)

### General Procedure for Preparation of Intermediate 3:

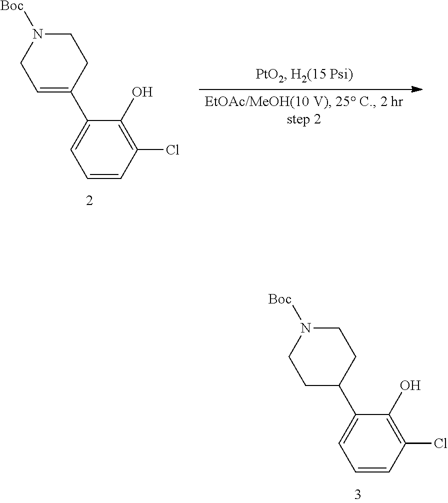 [SMILES: CC(C)(C)OC(=O)N1CC=C(c2cccc(Cl)c2O)CC1 ; "EtOAc/MeOH(10 V), 25° C., 2 hrstep 2" ; "PtO2, H2(15 Psi)" ; CC(C)(C)OC(=O)N1CCC(c2cccc(Cl)c2O)CC1]

Equip a 50 mL round bottom flask, and H₂ balloon. PtO₂ (100 mg) was charged to the 50 mL round bottom flask under Ar atmosphere, then EtOAc 6 mL was added at 25° C. 2 (790 mg, 2.55 mmol, 1 eq) in MeOH 3 mL was added to the reaction mixture at 25° C. After the addition, the mixture was stirred at 25° C. for 12 hr under H₂ atmosphere (15 Psi). The reaction was monitored by LCMS. LC-MS (product RT=1.983) showed 8% of 2 remained and one main peak with desired mass was detected. The reaction mixture was filtered through a pad of Celite, and the filter cake was washed with EtOAc 100 mL. Then the organic phase was concentrated under reduced pressure at 45° C. to give a residue. The residue was purified by column chromatography (SiO₂, Petroleum ether/Ethyl acetate=1/0 to 15/1). 3 (400 mg, 1.28 mmol, 50% yield) was obtained as a white solid.

LCMS: RT=1.970 min, MS cal.: 311.1, 313.1, [M−t-Bu+ACN]⁺=297.1, 299.1

¹H NMR (400 MHz, CHLOROFORM-d) δ=7.19 (dd, J=1.2, 8.0 Hz, 1H), 7.06 (dd, J=1.2, 8.0 Hz, 1H), 6.88-6.82 (m, 1H), 4.25 (d, J=13.2 Hz, 2H), 3.10 (tt, J=3.2, 12.0 Hz, 1H), 2.84 (dt, J=2.4, 12.8 Hz, 2H), 1.84 (d, J=12.8 Hz, 2H), 1.61 (d, J=4.0 Hz, 2H), 1.49 (s, 9H)

### General Procedure for Preparation of Intermediate 4:

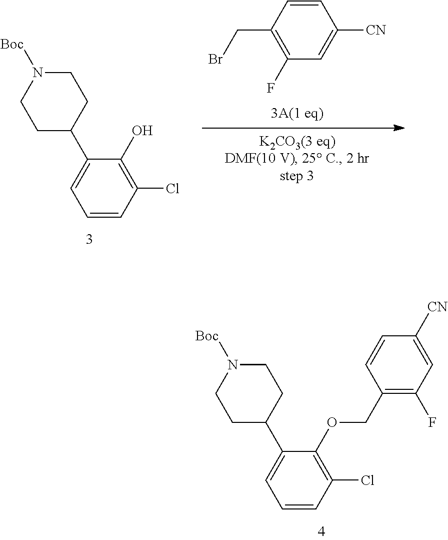

To a solution of 3 (400 mg, 1.28 mmol, 1 eq) in DMF (4 mL) was added 3A (274.58 mg, 1.28 mmol, 1 eq) and K₂CO₃ (531.90 mg, 3.85 mmol, 3 eq) was added. The resulting mixture was stirred at 25° C. for 2 hr. TLC (Petroleum ether/Ethyl acetate=10/1, product R_{f}=0.27) indicated 3 was consumed completely and one new spot formed. The reaction was clean according to TLC. The reaction mixture was partitioned between H₂O 20 mL and EtOAc 60 mL. The organic phase was separated, washed with brine 10 mL, dried over Na₂SO₄, filtered, and concentrated under reduced pressure to give a residue. The residue was purified by column chromatography (SiO₂, Petroleum ether/Ethyl acetate=50/1 to 10/1). 4 (470 mg, 1.06 mmol, 82% yield) was obtained as a colorless oil.

LCMS: RT=2.416 min, MS cal.: 444.2, 446.2, [M−t-Bu+ACN+H]⁺=430.2, 432.1

¹H NMR (400 MHz, CHLOROFORM-d) δ=7.83 (t, J=7.2 Hz, 1H), 7.56 (d, J=8.0 Hz, 1H), 7.43 (d, J=9.2 Hz, 1H), 7.30 (dd, J=2.0, 7.2 Hz, 1H), 7.17-7.07 (m, 2H), 5.09 (s, 2H), 4.24 (d, J=11.6 Hz, 2H), 3.04 (tt, J=3.2, 12.0 Hz, 1H), 2.73 (t, J=11.6 Hz, 2H), 1.74-1.67 (m, 2H), 1.65-1.59 (m, 1H), 1.57-1.52 (m, 1H), 1.48 (s, 9H)

### General Procedure for Preparation of Intermediate 5:

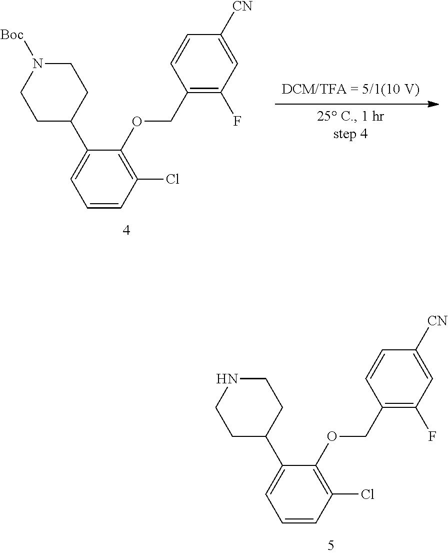

To a solution of 4 (200 mg, 449.51 μmol, 1 eq) in DCM (2 mL) was added TFA (614.00 mg, 5.38 mmol, 0.4 mL, 11.98 eq). The mixture was stirred at 25° C. for 1 hr. TLC (Petroleum ether/Ethyl acetate=10/1) indicated 4 was consumed completely and one new spot formed. The reaction was clean according to TLC. The reaction mixture was concentrated under reduced pressure to give 5 (200 mg, crude) as colorless oil.

LCMS: RT=1.334 min, MS cal.: 344.1, 346.1, [M+H]⁺=345.1, 347.1

¹H NMR (400 MHz, CHLOROFORM-d) δ=7.80 (t, J=7.6 Hz, 1H), 7.58 (d, J=8.0 Hz, 1H), 7.46 (d, J=9.6 Hz, 1H), 7.37 (dd, J=3.2, 6.0 Hz, 1H), 7.20-7.15 (m, 2H), 5.09 (s, 2H), 3.62 (d, J=12.4 Hz, 2H), 3.30-3.22 (m, 2H), 3.13-2.98 (m, 2H), 2.07-1.97 (m, 4H)

### General Procedure for Preparation of Intermediate 6:

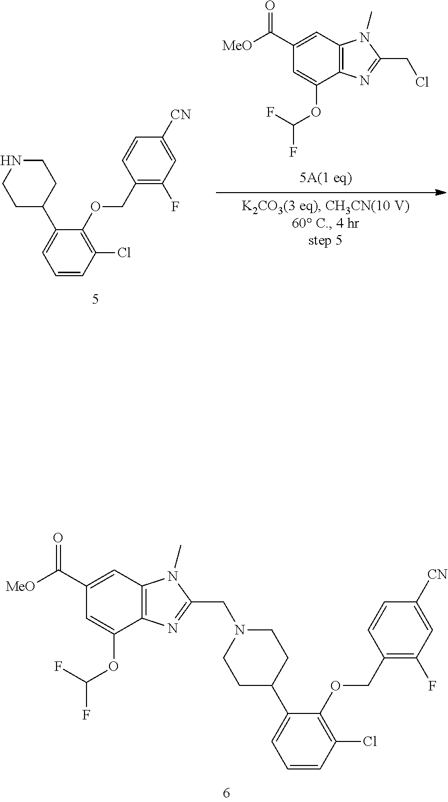 [SMILES: N#Cc1ccc(COc2c(Cl)cccc2C2CCNCC2)c(F)c1 ; COC(=O)c1cc(OC(F)F)c2nc(CCl)n(C)c2c1 ; "5A(1 eq)" ; "K2CO3(3 eq), CH3CN(10 V)60° C., 4 hrstep 5" ; COC(=O)c1cc(OC(F)F)c2nc(CN3CCC(c4cccc(Cl)c4OCc4ccc(C#N)cc4F)CC3)n(C)c2c1]

To a solution of 5 (200 mg, 435.89 μmol, 1 eq, TFA) in ACN (2 mL) was added 5A (132.81 mg, 435.89 μmol, 1 eq) and K₂CO₃ (180.73 mg, 1.31 mmol, 3 eq). The mixture was stirred at 60° C. for 2 hr. TLC (Petroleum ether/Ethyl acetate=1/1, product R_{f}=0.24) indicated 5 was consumed completely and one new spot formed. The reaction was clean according to TLC. The reaction mixture was diluted with H₂O 20 mL and extracted with EtOAc 60 mL (30 mL*2). The combined organic layers were washed with brine 20 mL, dried over Na₂SO₄, filtered and concentrated under reduced pressure to give a residue. The crude product was triturated with Petroleum ether/Ethyl acetate=1/1 (5 mL) at 25° C. for 5 min. The mixture was filtered, and the filter cake was washed with EtOAc 20 mL, dried in vacuum to give product. 6 (220 mg, 358.88 μmol, 82% yield) was obtained as a white solid.

LCMS: RT=1.749 min, MS cal.: 612.2, 614.2, [M+H]⁺=613.2, 615.2

¹H NMR (400 MHz, DMSO-d6) δ=8.24 (s, 1H), 8.07 (d, J=1.2 Hz, 1H), 7.87 (d, J=9.6 Hz, 1H), 7.82-7.79 (m, 1H), 7.76-7.74 (m, 1H), 7.62 (t, J=74.4 Hz, 1H), 7.32 (dd, J=1.6, 8.0 Hz, 1H), 7.25-7.20 (m, 1H), 7.16-7.10 (m, 1H), 5.06 (s, 2H), 3.96 (s, 3H), 3.90 (s, 3H), 3.84 (s, 2H), 2.92 (d, J=10.8 Hz, 2H), 2.80-2.85 (m, 1H), 2.12 (dt, J=4.0, 11.6 Hz, 2H), 1.66-1.56 (m, 4H)

### General Procedure for Preparation of Compound 138 (7):

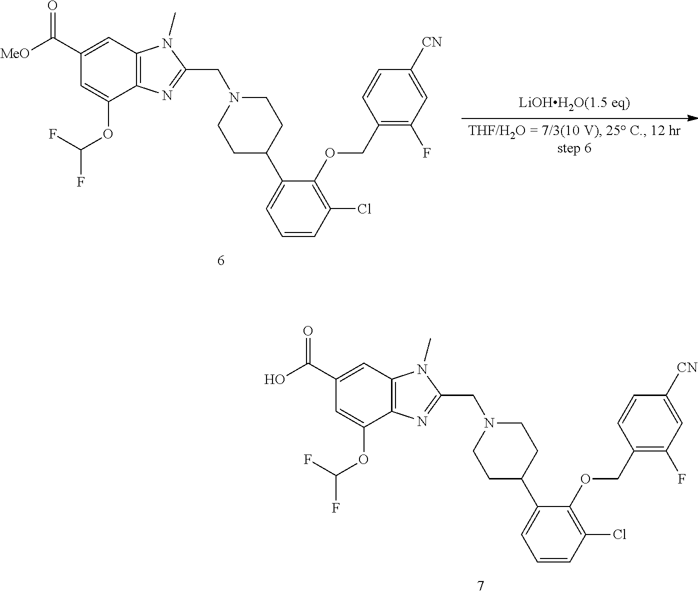

To a solution of 6 (120 mg, 195.75 μmol, 1 eq) in THF (0.7 mL) was added LiOH H₂O (12.32 mg, 293.63 μmol, 1.5 eq) in H₂O (0.3 mL). The mixture was stirred at 25° C. for 2 hr. LC-MS (product RT=1.127) showed 6 was consumed completely and desired mass was detected. The reaction mixture was purified directly. The residue was purified by prep-HPLC (neutral condition, column: Waters Xbridge Prep OBD C18 150*40 mm*10 um; mobile phase: [H₂O (10 mM NH₄HCO₃)-ACN]; gradient: 10%-60% B over 8.0 min). 2-((4-(3-Chloro-2-((4-cyano-2-fluorobenzyl)oxy)phenyl)piperidin-1-yl)methyl)-4-(difluoromethoxy)-1-methyl-1H-benzo[d]imidazole-6-carboxylic acid (29.39 mg, 47.90 μmol, 19% yield, 97.63% purity) was obtained as a white solid.

LCMS: RT=2.311 min, MS cal.: 598.2, 600.2, [M+H]⁺=599.2, 601.2

HPLC: RT=11.137 min, purity: 97.63%

¹H NMR (400 MHz, METHANOL-d4) δ=8.10 (s, 1H), 7.85-7.79 (m, 1H), 7.67 (d, J=4.8 Hz, 2H), 7.65 (d, J=4.0 Hz, 1H), 7.30 (dd, J=1.2, 7.6 Hz, 1H), 7.24 (t, J=74.0 Hz, 1H), 7.23 (d, J=6.4 Hz, 1H), 7.15-7.09 (m, 1H), 5.12 (s, 2H), 4.00 (s, 3H), 3.93 (s, 2H), 3.04 (d, J=11.2 Hz, 2H), 2.99-2.89 (m, 1H), 2.26 (dt, J=2.4, 11.6 Hz, 2H), 1.81-1.64 (m, 4H)

### Example A270—Preparation of Compound 137 and Compound 185. 2-((4-(6-((4-Cyano-2-fluorobenzyl)oxy)-5-fluoropyridin-2-yl)piperidin-1-yl)methyl)-4-(difluoromethoxy)-1-methyl-1H-benzo[d]imidazole-6-carboxylic acid (Compound 137) (7) & 2-((4-(6-((4-carbamoyl-2-fluorobenzyl)oxy)-5-fluoropyridin-2-yl)piperidin-1-yl)methyl)-4-(difluoromethoxy)-1-methyl-1H-benzo[d]imidazole-6-carboxylic acid (Compound 185) (8)

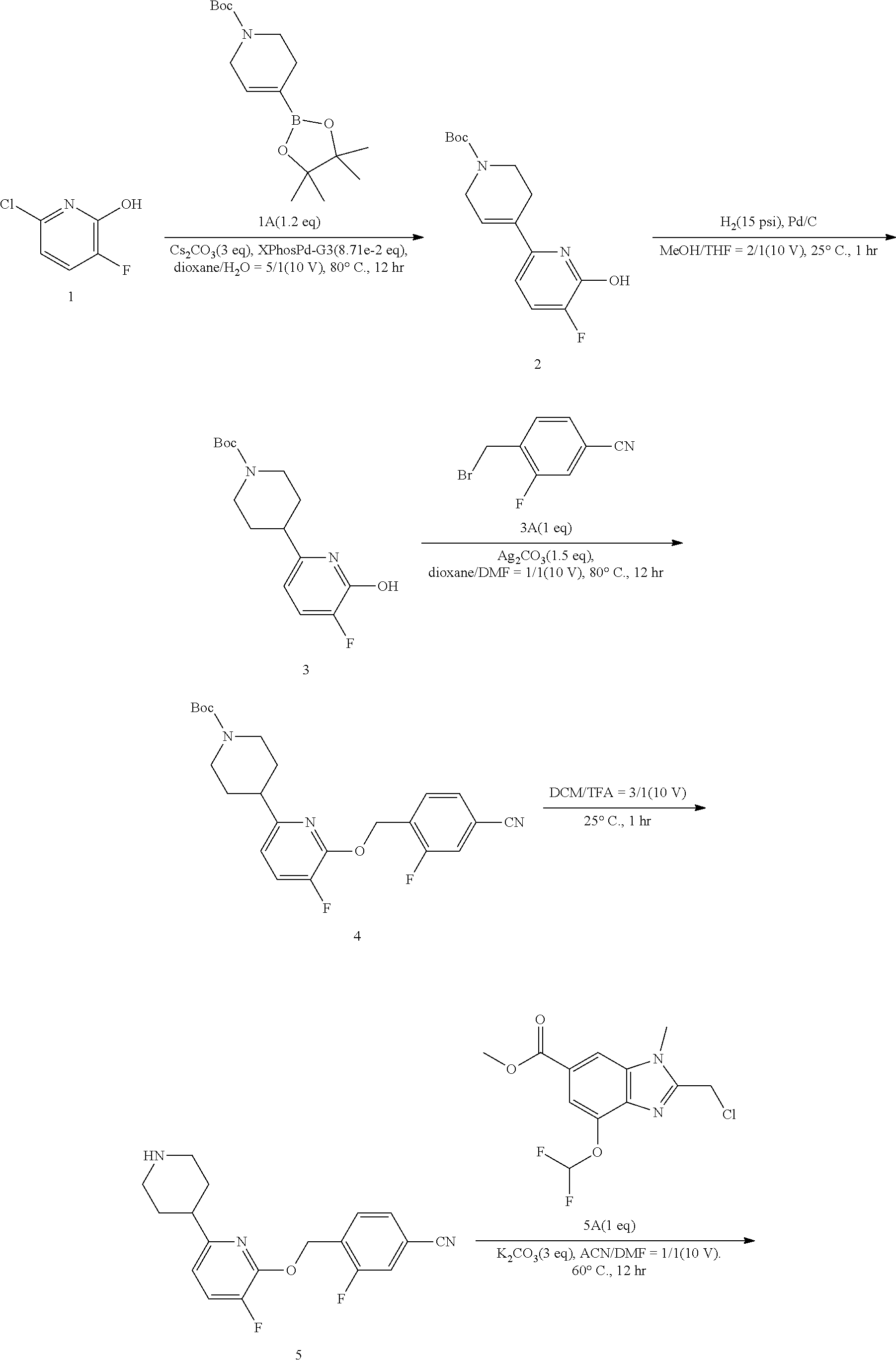

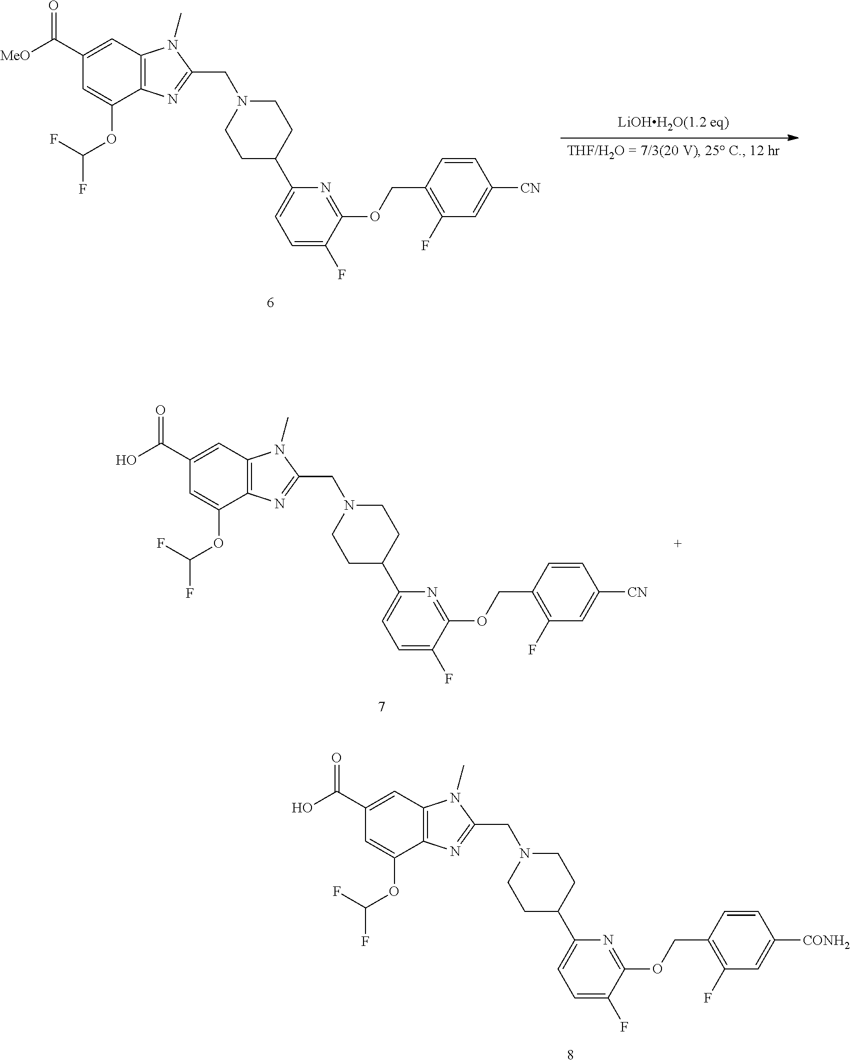

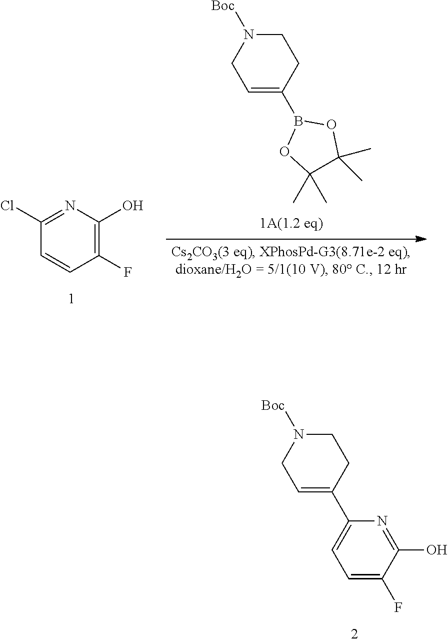

To a solution of 1A (22.63 g, 73.20 mmol, 1.2 eq) and 1 (9 g, 61.00 mmol, 1 eq) in H₂O (37.7 mL) and dioxane (188.5 mL) was added Cs₂CO₃ (59.63 g, 183.01 mmol, 3 eq) and XPhos Pd G₃ (4.50 g, 5.32 mmol, 8.71e-2 eq). The mixture was stirred at 80° C. for 12 hr. LC-MS showed 1 was consumed completely and desired mass was detected. The reaction mixture was filtered through celite pad. The filtrate was diluted with H₂O (20 mL) and extracted with EtOAc (30 mL*3). The combined organic layers were washed with brine (20 mL), dried over Na₂SO₄, filtered and concentrated under reduced pressure to give a residue. The crude product was triturated with EtOAc (20 mL) at 25° C. for 10 min. Filtered and the filter cake was washed with MTBE (10 mL*3). The filter cake was dried under vacuum. 2 (14.6 g, 49.61 mmol, 81.32% yield) was obtained as a white solid.

LCMS: RT=1.057 min, MS cal.: 294.3, [M−H]⁻=293.1

¹H NMR (400 MHz, DMSO-d6) δ=11.86 (br d, J=3.2 Hz, 1H), 7.36 (dd, J=7.6, 10.4 Hz, 1H), 6.39 (s, 1H), 6.18 (m, 1H), 3.98 (m, 2H), 3.47 (t, J=5.2 Hz, 2H), 2.40-2.32 (m, 2H), 1.41 (s, 9H)

### General Procedure for Preparation of Intermediate 3:

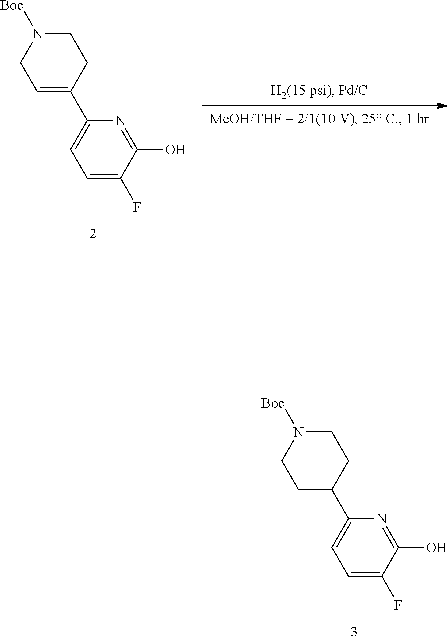

MeOH (84 mL) and THF (42 mL) were charged to the round bottom flask, then 2 (12 g, 40.77 mmol, 1 eq) was added to the mixture at 25° C. At 25° C., Pd/C (2 g) was added to the mixture under N₂ atmosphere. The suspension was degassed and purged with H₂ for 3 times. The mixture was stirred under H₂ (15 Psi) at 25° C. for 1 hr. LC-MS showed 2 was consumed completely and desired mass was detected. The mixture was filtered through a Celite pad and washed by MeOH (20 mL*3). And the filtrate was concentrated to give the crude product. 3 (12 g, 40.49 mmol, 99.32% yield) was obtained as a white solid.

LCMS: RT=1.017 min, MS cal.: 296.3, [M−H]⁺=295.0

¹H NMR (400 MHz, DMSO-d6) δ=7.28 (dd, J=7.6, 10.8 Hz, 1H), 5.94 (d, J=3.2 Hz, 1H), 4.04 (d, J=10.8 Hz, 2H), 2.80-2.65 (m, 2H), 2.62-2.53 (m, 1H), 1.81-1.70 (m, 2H), 1.45-1.42 (m, 2H), 1.40 (s, 9H)

### General Procedure for Preparation of Intermediate 4:

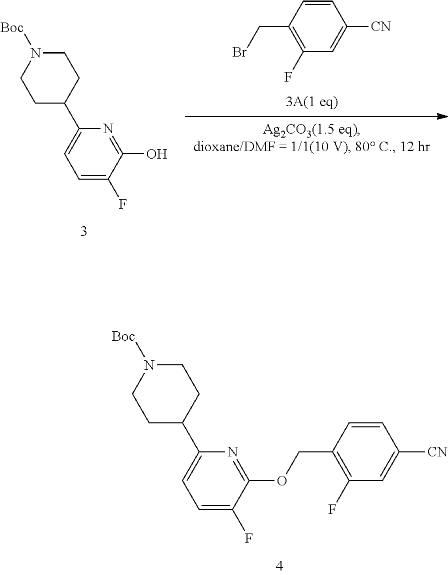

To a solution of 3 (12 g, 40.49 mmol, 1 eq) and 3A (8.67 g, 40.49 mmol, 1 eq) in dioxane (60 mL) and DMF (60 mL) was added Ag₂CO₃ (16.75 g, 60.74 mmol, 2.76 mL, 1.5 eq). The mixture was stirred at 80° C. for 12 hr. LC-MS showed 3 was consumed completely and desired mass was detected. The suspension was filtered through a pad of Celite, and the filter cake was washed with EtOAc (20 ml*3). The filtrate was diluted with H₂O (20 mL) and extracted by EtOAc (30 mL*2). Then organic phase was combined and washed by brine (20 mL), dried with Na₂SO₄, filtered. The filtrate was concentrated under reduced pressure at 40° C. to give residue. The residue was purified by column chromatography (SiO₂, Petroleum ether/Ethyl acetate=100/1 to 3/1). 4 (14 g, 32.60 mmol, 80.50% yield) was obtained as a white solid.

LCMS: RT=0.910 min, MS cal.: 429.4, [M+Na]⁺=452.2

¹H NMR (400 MHz, DMSO-d6) δ=7.87 (d, J=10.4 Hz, 1H), 7.73-7.70 (m, 2H), 7.62 (dd, J=8.0, 10.8 Hz, 1H), 6.92 (dd, J=2.8, 8.4 Hz, 1H), 5.54 (s, 2H), 4.07-3.95 (m, 2H), 2.86-2.70 (m, 3H), 1.73-1.71 (m, 2H), 1.53-1.44 (m, 2H), 1.41 (s, 9H)

### General Procedure for Preparation of Intermediate 5:

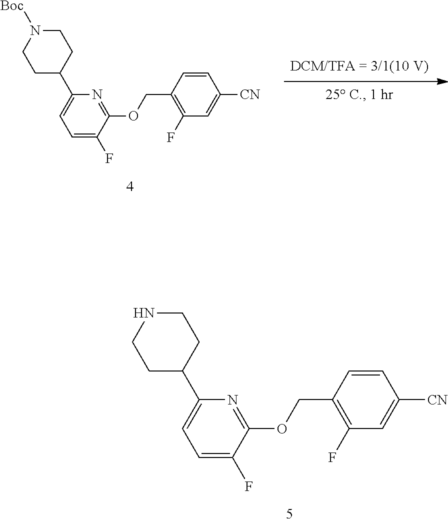

To a solution of 4 (14.5 g, 33.76 mmol, 1 eq) in DCM (108.75 mL) was added TFA (55.64 g, 488.00 mmol, 36.25 mL, 14.45 eq). The mixture was stirred at 25° C. for 1 hr. LC-MS showed 4 was consumed completely and desired mass was detected. The organic phase was added with sat. Na₂CO₃ adjusted to pH=7˜8. Then the mixture was diluted with H₂O (50 mL) and extracted by DCM (50 mL*2). Then organic phase was combined and washed by brine (30 mL), dried with Na₂SO₄, filtered. The filtrate was concentrated under reduced pressure at 40° C. to give residue. 5 (11 g, 33.40 mmol, 98.92% yield) was obtained as a white solid.

LCMS: RT=0.627 min, MS cal.: 329.3, [M+H]⁺=330.1

¹H NMR (400 MHz, DMSO-d6) δ=7.91 (d, J=10.0 Hz, 1H), 7.75-7.71 (m, 2H), 7.62 (dd, J=8.0, 10.4 Hz, 1H), 6.88 (dd, J=2.8, 8.0 Hz, 1H), 5.55 (s, 2H), 3.06 (d, J=12.0 Hz, 2H), 2.73-2.59 (m, 3H), 1.73 (d, J=11.2 Hz, 2H), 1.62-1.49 (m, 2H)

### General Procedure for Preparation of Intermediate 6:

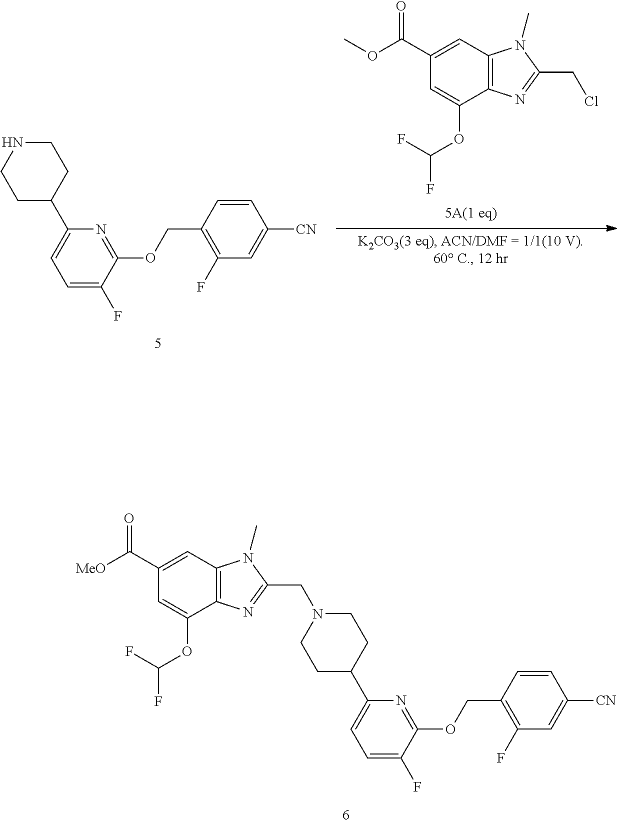

To a solution of 5 (7 g, 21.25 mmol, 1 eq) and 5A (6.48 g, 21.25 mmol, 1 eq) in DMF (35 mL) and ACN (35 mL) was added K₂CO₃ (8.81 g, 63.76 mmol, 3 eq). The mixture was stirred at 60° C. for 12 hr. LC-MS showed 5 was consumed completely and desired mass was detected. The reaction mixture was quenched by addition H₂O (350 mL) at 25° C. The crude product was triturated with H₂O (70 mL) at 25° C. for 10 min. Filtered and the filter cake was triturated with ACN (70 mL) at 25° C. for 10 min. Filtered and the filter cake was washed with ACN (30 mL*3). The filter cake was dried under vacuum. 6 (10 g, 16.73 mmol, 78.74% yield) was obtained as a white solid.

LCMS: RT=0.458 min, MS cal.: 597.5, [M+H]⁺=598.2

¹H NMR (400 MHz, DMSO-d6) δ=8.12 (d, J=1.2 Hz, 1H), 7.91 (d, J=10.4 Hz, 1H), 7.72 (d, J=3.6 Hz, 2H), 7.65 (t, J=74.4 Hz, 1H), 7.61 (dd, J=8.0, 10.4 Hz, 1H), 7.53 (s, 1H), 6.91 (dd, J=2.8, 8.0 Hz, 1H), 5.54 (s, 2H), 3.98 (s, 3H), 3.90 (s, 3H), 3.86 (s, 2H), 2.96-2.89 (m, 2H), 2.64-2.54 (m, 1H), 2.21 (t, J=10.4 Hz, 2H), 1.78-1.71 (m, 2H), 1.70-1.56 (m, 2H)

### General Procedure for Preparation of Compound 137 (7):

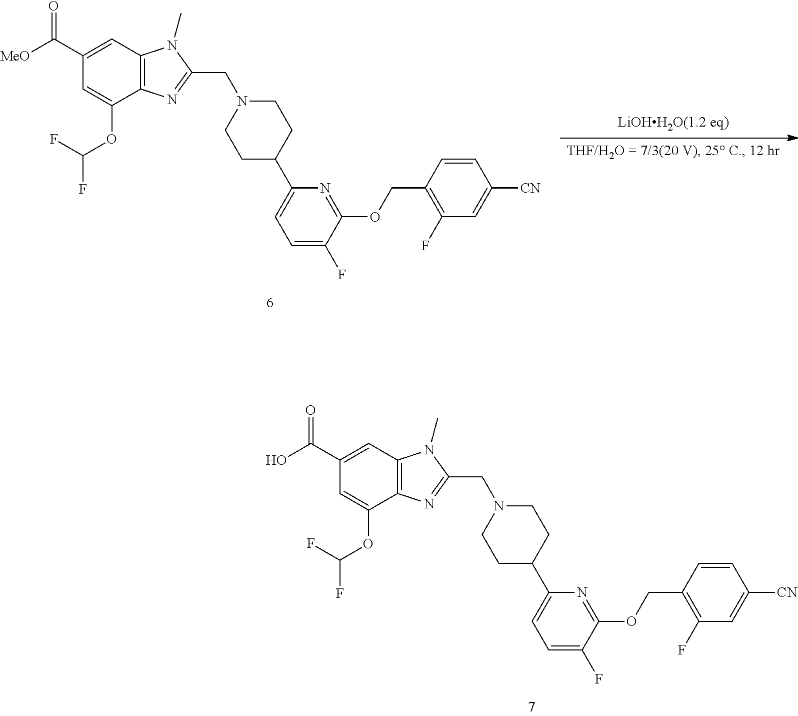

To a solution of 6 (10 g, 16.73 mmol, 1 eq) in THF (140 mL) and H₂O (60 mL) was added LiOH·H₂O (1 M, 20.08 mL, 1.2 eq). The mixture was stirred at 25° C. for 12 hr. LC-MS showed 6 was consumed completely and desired mass was detected. The mixture was diluted with H₂O (40 mL) and extracted by MTBE (20 mL*5). The aqueous layer was added with sat. FA (1 M) adjusted to pH-4˜5, then solid was formed. Filtered and the filter cake was washed with H₂O (20 mL*2). The filter cake was dried under vacuum. The crude product was purified by reversed-phase HPLC (column: Phenomenex luna c18 250 mm*100 mm*15 um; mobile phase: [H₂O (0.2% FA)-ACN]; gradient: 20%-50% B over 22.0 min). 2-((4-(6-((4-Cyano-2-fluorobenzyl)oxy)-5-fluoropyridin-2-yl)piperidin-1-yl)methyl)-4-(difluoromethoxy)-1-methyl-1H-benzo[d]imidazole-6-carboxylic acid (5.8 g, 9.94 mmol, 59.39% yield) was obtained as a white solid.

LCMS: RT=3.058 min, MS cal.: 583.5, [M+H]⁺=584.2

HPLC: RT=11.126 min, purity: 97%

¹H NMR (400 MHz, METHANOL-d4) δ=8.12 (d, J=0.8 Hz, 1H), 7.74-7.66 (m, 2H), 7.62-7.54 (m, 2H), 7.41 (dd, J=8.0, 10.0 Hz, 1H), 7.27 (d, J=74.4 Hz, 1H), 6.85 (dd, J=2.8, 8.0 Hz, 1H), 5.59 (s, 2H), 4.02 (s, 3H), 4.01 (s, 2H), 3.12 (d, J=11.6 Hz, 2H), 2.73-2.63 (m, 1H), 2.44 (dt, J=3.2, 10.8 Hz, 2H), 1.92-1.78 (m, 4H)

### General Procedure for Preparation of Compound 185 (8):

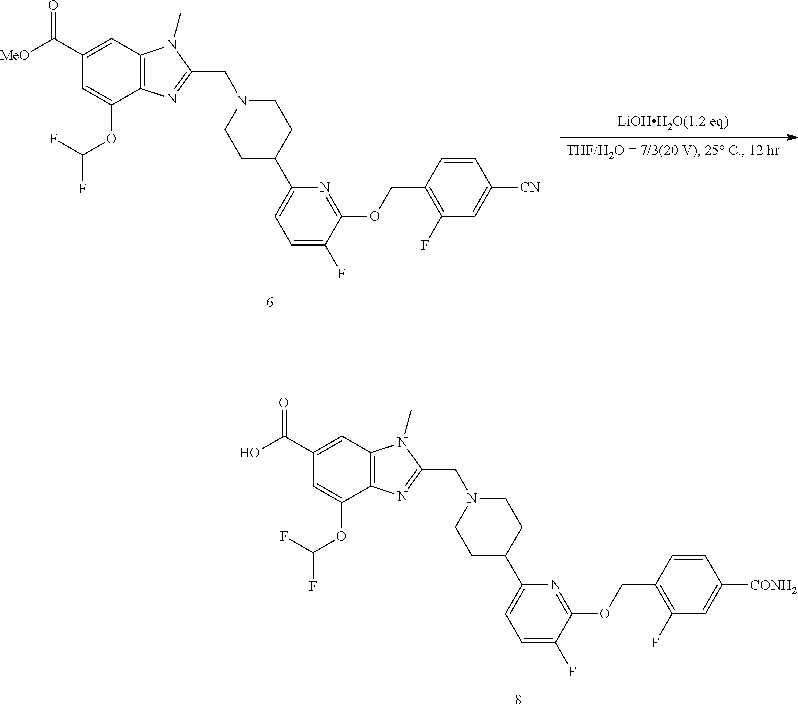

To a solution of 6 (500 mg, 836.74 μmol, 1 eq) in THF (7 mL) and H₂O (3 mL) was added LiOH·H₂O (35.11 mg, 836.74 μmol, 1 eq). The mixture was stirred at 25° C. for 6 hr. LC-MS showed 6 remained, LiOH·H₂O (1 M, 167.35 μL, 0.2 eq) was added to the reaction mixture. After the addition, the mixture was stirred at 25° C. for 6 hr. LC-MS showed 6 was consumed completely and desired mass was detected. The mixture was diluted with H₂O (1.5 mL) and extracted by PE (5 mL*5). The aqueous phase was added with FA (1 M) adjust to pH=4˜5. The crude product was purified by prep-HPLC (column: Waters Xbridge Prep OBD C18 150*40 mm*10 um; mobile phase: [H₂O (10 mM NH₄HCO₃)-ACN]; gradient: 25%-45% B over 8.0 min). 2-((4-(6-((4-Carbamoyl-2-fluorobenzyl)oxy)-5-fluoropyridin-2-yl)piperidin-1-yl)methyl)-4-(difluoromethoxy)-1-methyl-1H-benzo[d]imidazole-6-carboxylic acid (11.78 mg, 19.58 μmol, 2.34% yield) was obtained as a white solid.

LCMS: RT=2.412 min, MS cal.: 601.5, [M+H]⁺=602.2

HPLC: RT=8.865 min, purity: 99%

¹H NMR (400 MHz, METHANOL-d₄) δ=8.11 (s, 1H), 7.70-7.57 (m, 4H), 7.39 (dd, J=8.2, 10.3 Hz, 1H), 7.24 (d, J=74.4 Hz, 1H), 6.82 (dd, J=2.8, 8.0 Hz, 1H), 5.57 (s, 2H), 4.02 (s, 3H), 3.95 (s, 2H), 3.05 (d, J=10.8 Hz, 2H), 2.70-2.61 (m, 1H), 2.41-2.32 (m, 2H), 1.90-1.81 (m, 4H)

### Example A271-Preparation of Compound 142 and Compound 133. 2-(((1s,4s)-4-(6-((4-Cyano-2-fluorobenzyl)oxy)pyridin-2-yl)cyclohexyl)(methyl)amino)-4-(difluoromethoxy)-1-methyl-1H-benzo[d]imidazole-6-carboxylic acid (Compound 142) (5) & 2-(((1r,4r)-4-(6-((4-Cyano-2-fluorobenzyl)oxy)pyridin-2-yl)cyclohexyl)(methyl)amino)-4-(difluoromethoxy)-1-methyl-1H-benzo[d]imidazole-6-carboxylic acid (Compound 133) (5B)

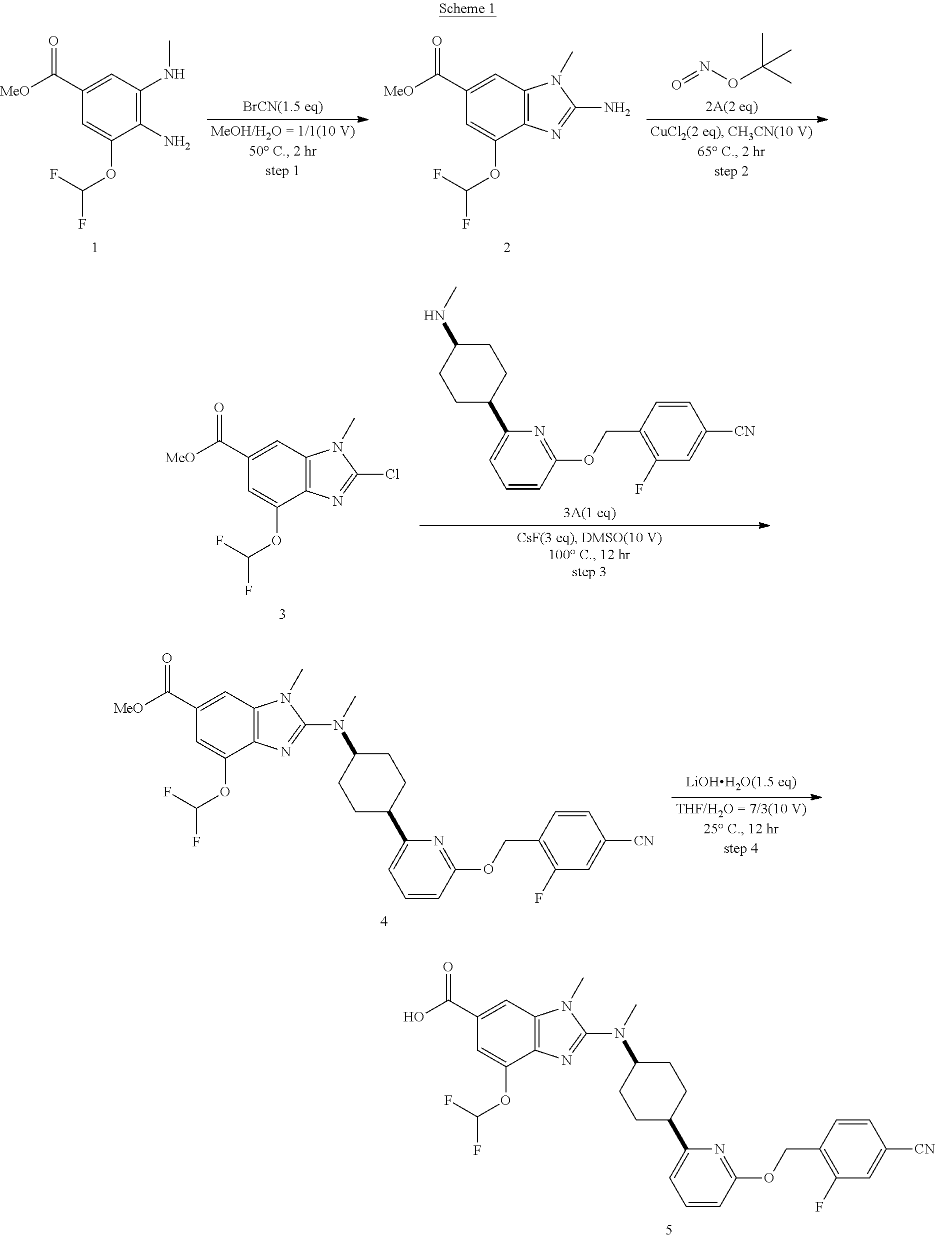

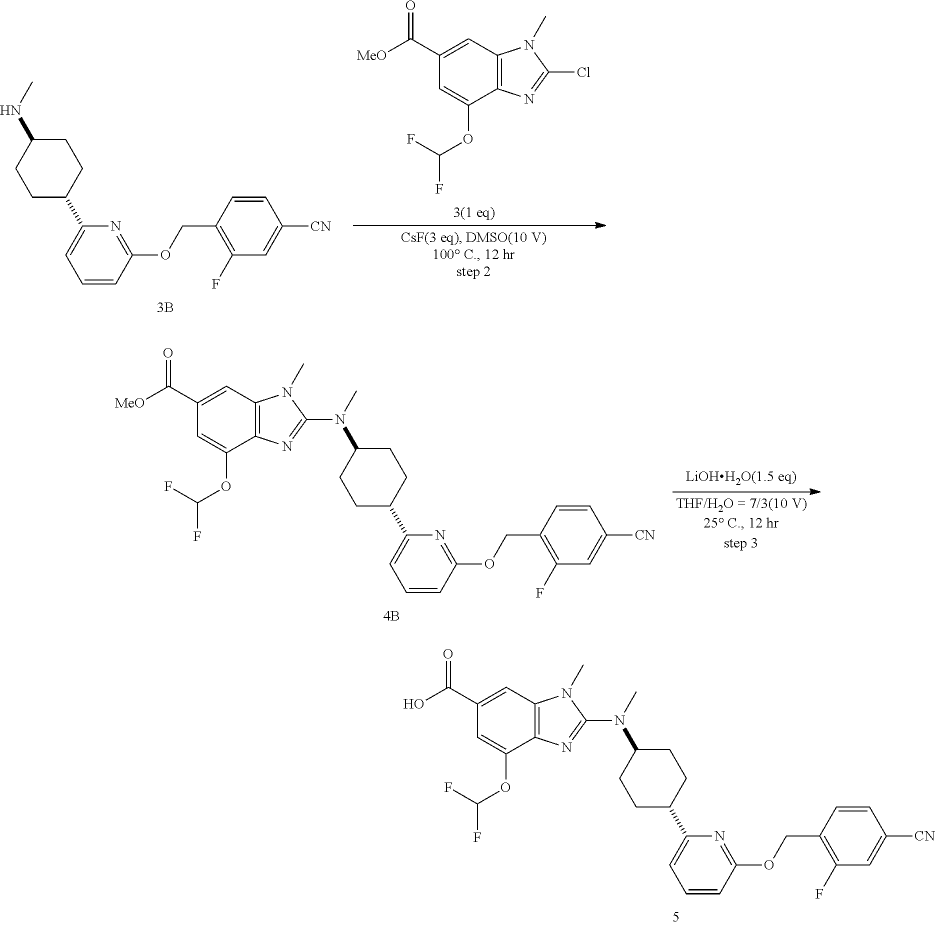

### General Procedure for Preparation of Intermediate 2:

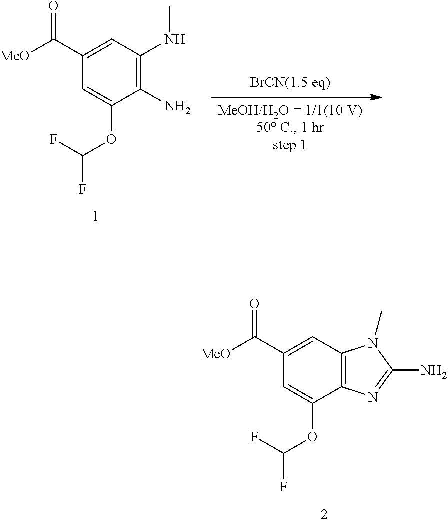

Equip a 25 mL three-necked round bottom flask, thermometer, N₂ balloon. MeOH (5 mL) and H₂O (5 mL) were charged to the 25 mL three-necked round bottom flask, then 1 (500 mg, 2.03 mmol, 1 eq) was added to the mixture at 20° C. At 20° C. (inner temperature), BrCN (322.66 mg, 3.05 mmol, 223.60 μL, 1.5 eq) was added dropwise to the reaction mixture at 20° C. After the addition, the mixture was stirred at 50° C. for 2 hr. LCMS showed 1 was consumed completely and one main peak with desired mass was detected. The reaction mixture was adjusted to pH=8 with 1M NaHCO₃ at 20° C., and then diluted with H₂O (10 mL) and extracted with EtOAc (20 mL*2). The combined organic layers were washed with brine 20 mL, dried over Na₂SO₄, filtered, and concentrated under reduced pressure to give a residue. Without purification. 2 (530 mg, 1.93 mmol, 95.26% yield) was obtained as a brown solid.

LCMS: RT=0.301 min, MS cal.: 271.1/272.1, [M+H]⁺=272.0/273.0

¹HNMR (400 MHz, CHLOROFORM-d) δ=7.74-7.72 (m, 1H), 7.68-7.66 (m, 1H), 7.07 (t, J=74.8 Hz, 1H), 3.94 (s, 3H), 3.62-3.62 (m, 1H), 3.62 (s, 2H)

### General Procedure for Preparation of Intermediate 3:

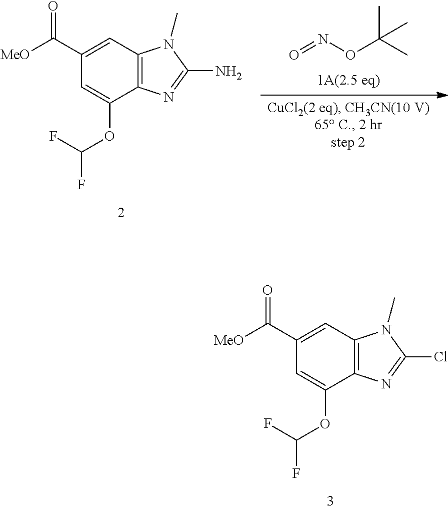

To a solution of 2 (280 mg, 1.03 mmol, 1 eq) in CH₃CN (3 mL) was added CuCl₂ (277.61 mg, 2.06 mmol, 2 eq) and tert-butyl nitrite (212.92 mg, 2.06 mmol, 245.58 μL, 2 eq). The mixture was stirred at 65° C. for 2 hr. LCMS showed 2 was consumed completely. Several new peaks were shown on LCMS and 68% of desired 3 was detected. The reaction mixture was diluted with H₂O 10 mL and extracted with EtOAc 20 mL (10 mL*2). The combined organic layers were washed with brine 10 mL, dried over Na₂SO₄, filtered and concentrated under reduced pressure to give a residue. The residue was purified by column chromatography (SiO₂, PE/EtOAc=1/0 to 4/1). 3 (190 mg, 647.17 μmol, 62.69% yield) was obtained as a yellow solid.

LCMS: RT=0.459 min, MS cal.: 290.2/292.0, [M+H]⁺=290.9/292.9

¹HNMR (400 MHz, CHLOROFORM-d) δ=7.94 (s, 1H), 7.76 (s, 1H), 7.26 (t, J=74 Hz, 1H), 3.98 (s, 3H), 3.87 (s, 3H)

### General Procedure for Preparation of Intermediate 4:

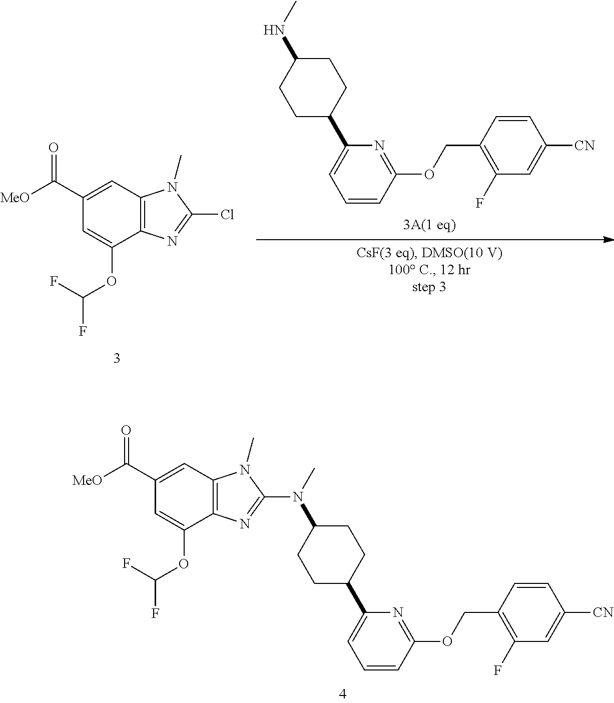

To a solution of 3 (100 mg, 344.06 μmol, 1 eq) in DMSO (1.5 mL) was added CsF (156.79 mg, 1.03 mmol, 3 eq) and 3A (116.77 mg, 344.06 μmol, 1 eq). The mixture was stirred at 100° C. for 12 hr. LCMS showed 3 was consumed completely. Several new peaks were shown on LCMS and 38% of desired 4 was detected. The reaction mixture was diluted with H₂O 5 mL and extracted with EtOAc 10 mL (5 mL*2). The combined organic layers were washed with brine 5 mL, dried over Na₂SO₄, filtered, and concentrated under reduced pressure to give a residue. The residue was purified by column chromatography (SiO₂, PE/EtOAc=1/0 to 1/1). 4 (70 mg, 109.67 μmol, 31.88% yield) was obtained as a white solid.

LCMS: RT=0.563 min, MS cal.: 593.2/594.2, [M+H]⁺=594.2/595.2

### General Procedure for Preparation of Compound 142 (5):

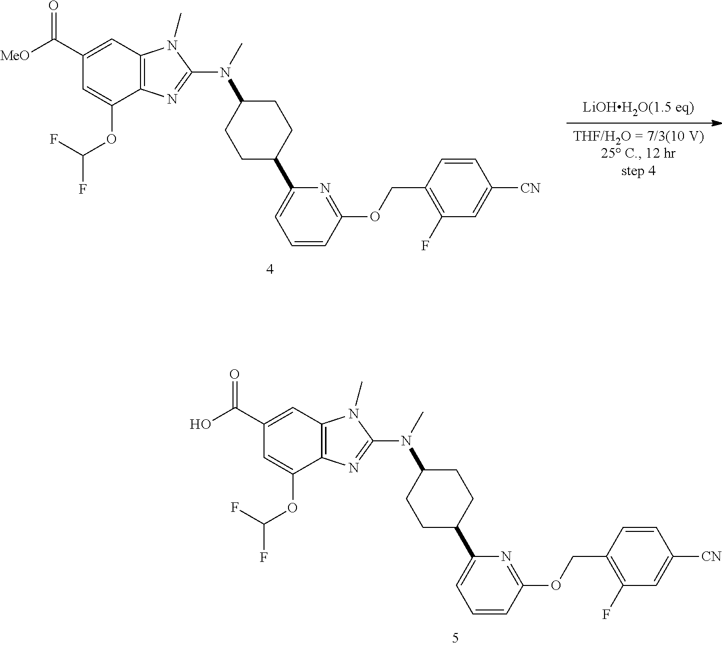

To a solution of 4 (70 mg, 117.93 μmol, 1 eq) in THF (0.7 mL) and H₂O (0.3 mL) was added LiOH·H₂O (7.42 mg, 176.89 μmol, 1.5 eq). The mixture was stirred at 25° C. for 2 hr. The mixture was added LiOH·H₂O (1.48 mg, 35.38 μmol, 0.3 eq). The mixture was stirred at 25° C. for 12 hr. The mixture was added LiOH·H₂O (2.47 mg, 58.96 μmol, 0.5 eq). The mixture was stirred at 25° C. for 12 hr. LCMS showed 84% of 4 remained. Several new peaks were shown on LCMS and 11% of desired product was detected. LCMS showed 70% of 4 remained. Several new peaks were shown on LCMS and 25% of desired product was detected. LCMS showed 36% of 4 remained. Several new peaks were shown in LCMS and 56% of desired product was detected. The mixture was added CH₃CN 0.2 mL. The residue was purified by prep-HPLC (neutral condition: column: Waters Xbridge Prep OBD C18 150*40 mm*10 um; mobile phase: [H₂O (10 mM NH₄HCO₃)-ACN]; gradient: 30%-60% B over 8.0 min). The residue was purified by prep-HPLC (FA condition: column: column: Phenomenex Luna C18 100*30 mm*3 um; mobile phase: [H₂O (0.2% FA)-ACN]; gradient: 25%-65% B over 8.0 min). 2-(((1s,4s)-4-(6-((4-Cyano-2-fluorobenzyl)oxy)pyridin-2-yl)cyclohexyl)(methyl)amino)-4-(difluoromethoxy)-1-methyl-1H-benzo[d]imidazole-6-carboxylic acid (8.6 mg, 14.39 μmol, 12.21% yield) was obtained as a white solid.

LCMS: RT=2.793 min, MS cal.: 579.2/580.2, [M+H]⁺=580.2/581.2

HPLC: RT=12.778 min

¹H NMR (400 MHz, DMSO-d₆) δ=7.89 (s, 1H), 7.70-7.52 (m, 5H), 7.21 (t, J=74.8 Hz, 1H), 6.96 (d, J=7.6 Hz, 1H), 6.72 (d, J=8.0 Hz, 1H), 5.57 (s, 2H), 3.74 (s, 3H), 3.72-3.65 (m, 1H), 2.91 (quin, J=4.8 Hz, 1H), 2.81 (s, 3H), 2.27-2.18 (m, 2H), 2.01 (s, 2H), 1.81-1.72 (m, 2H), 1.67 (td, J=3.6, 12.8 Hz, 2H)

### General Procedure for Preparation of Intermediate 4B:

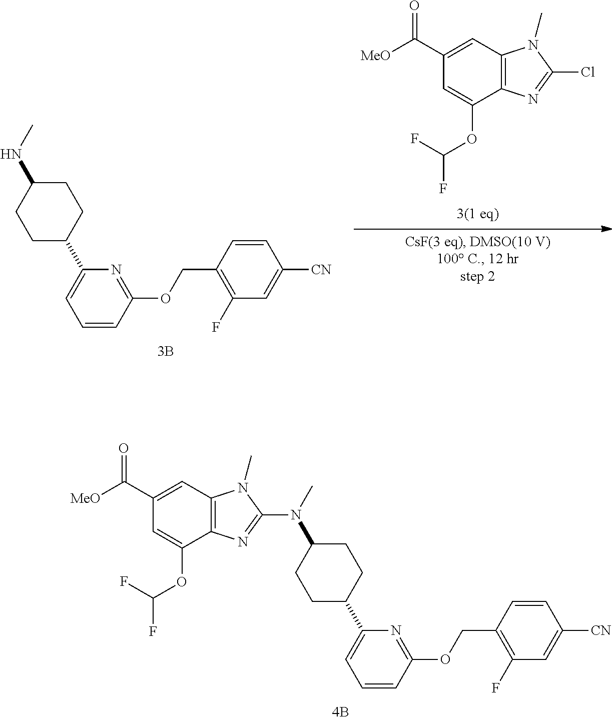

A mixture of 3 (100 mg, 344.06 μmol, 1 eq), 3B (116.77 mg, 344.06 μmol, 1 eq), CsF (156.79 mg, 1.03 mmol, 3 eq), in DMSO (1 mL) was degassed and purged with N₂ for 3 times, and then the mixture was stirred at 100° C. for 12 hr under N₂ atmosphere. LCMS (product: RT=1.678 min) showed 3B and 3 were consumed completely. The reaction mixture was quenched by addition H₂O 5 mL at 20° C. and extracted with EtOAc 5 mL*3. The combined organic layers were washed with brine 5 mL*2, dried over Na₂SO₄, filtered, and concentrated under reduced pressure to give residue. The residue was purified by column chromatography (SiO₂, Petroleum ether/Ethyl acetate=5/1 to 0/1). 4B (130 mg, 219.00 μmol, 63.65% yield) was obtained as a white solid.

¹H NMR (400 MHz, DMSO-d6) δ=7.90 (d, J=10.4 Hz, 1H), 7.83 (d, J=1.6 Hz, 1H), 7.74-7.69 (m, 2H), 7.65 (t, J=8.0 Hz, 1H), 7.60 (t, J=74.8, 1H), 7.46 (s, 1H), 6.88 (d, J=7.6 Hz, 1H), 6.71 (d, J=8.4 Hz, 1H), 5.47 (s, 2H), 3.87 (s, 3H), 3.71 (s, 3H), 3.70-3.63 (m, 1H), 2.97 (s, 3H), 2.59 (t, J=12.0 Hz, 1H), 1.91 (d, J=11.6 Hz, 4H), 1.85-1.75 (m, 2H), 1.69-1.54 (m, 2H)

### General Procedure for Preparation of Compound 133 (5B):

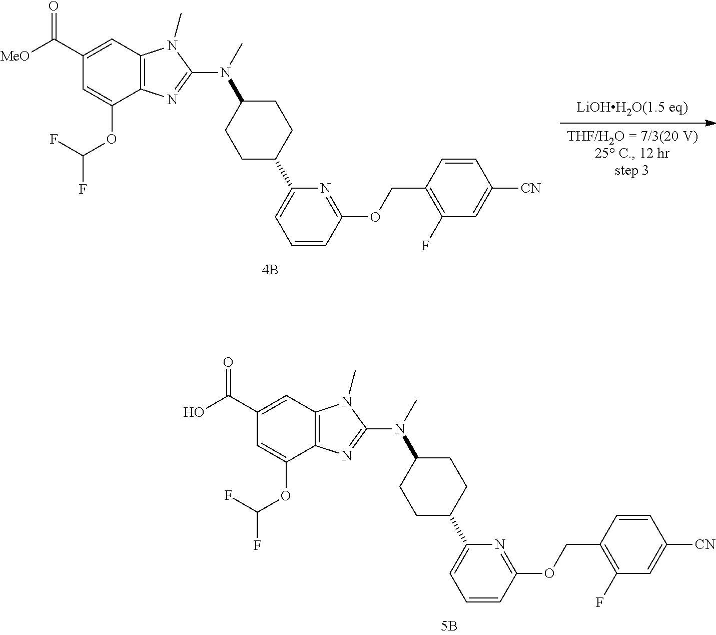

To a solution of 4B (100 mg, 168.47 μmol, 1 eq), in THF (1.4 mL) was added LiOH·H₂O (16.26 mg, 387.47 μmol, 2.3 eq), in H₂O (0.6 mL). The mixture was stirred at 25° C. for 12 hr under N₂ atmosphere. LCMS (product: RT=0.473 min) showed the 5 was consumed completely. The reaction was purified by prep-HPLC (column: Waters Xbridge BEH C18 100*30 mm 10 um; mobile phase: [H₂O (10 mM NH₄HCO₃)—CH₃CN]; gradient: 20%-65% B over 8.0 min). 2-(((1r,4r)-4-(6-((4-Cyano-2-fluorobenzyl)oxy)pyridin-2-yl)cyclohexyl)(methyl)amino)-4-(difluoromethoxy)-1-methyl-1H-benzo[d]imidazole-6-carboxylic acid (25.20 mg, 43.25 μmol, 25.67% yield) was obtained as a white solid.

LCMS: RT=2.814 min, MS cal.: 579.2, [M+H]⁺=580.2

HPLC: RT=12.547 min

¹H NMR (400 MHz, DMSO-d6) δ=7.90 (d, J=10.0 Hz, 1H), 7.79 (d, J=1.2 Hz, 1H), 7.75-7.68 (m, 2H), 7.65 (t, J=7.8 Hz, 1H), 7.58 (t, J=75.2 Hz, 1H), 7.45 (s, 1H), 6.88 (d, J=7.6 Hz, 1H), 6.71 (d, J=8.4 Hz, 1H), 5.47 (s, 2H), 3.69 (s, 3H), 3.65-3.57 (m, 1H), 2.94 (s, 3H), 2.59 (t, J=12.0 Hz, 1H), 1.91 (d, J=10.8 Hz, 4H), 1.86-1.74 (m, 2H), 1.69-1.52 (m, 2H)

### Example A272—Preparation of Compound 130. 2-((4-(6-((4-Cyano-2-fluorophenoxy)methyl)pyridin-2-yl)piperidin-1-yl)methyl)-4-(difluoromethoxy)-1-methyl-1H-benzo[d]imidazole-6-carboxylic acid (Compound 130) (6)

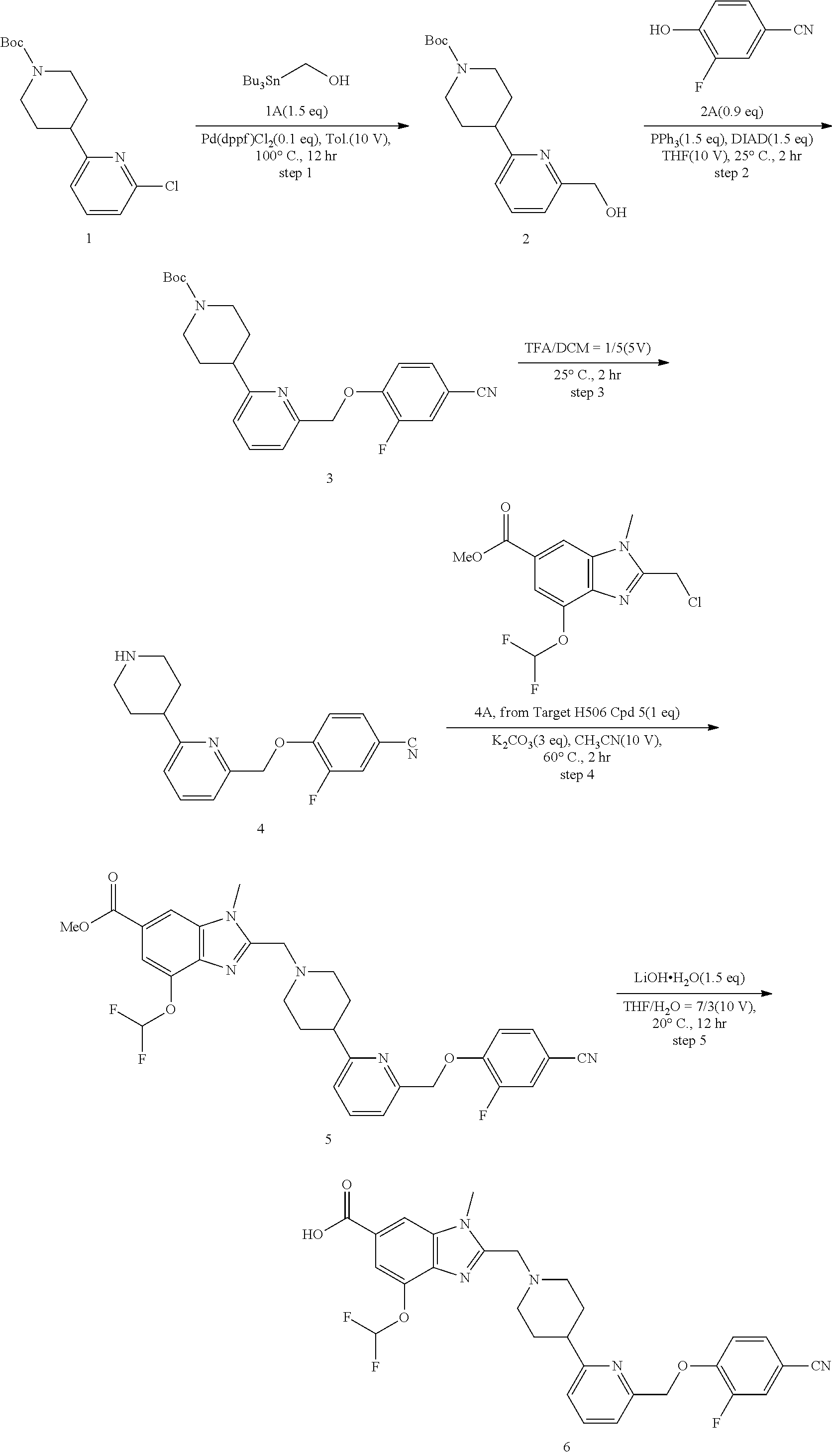

### General Procedure for Preparation of Intermediate 2:

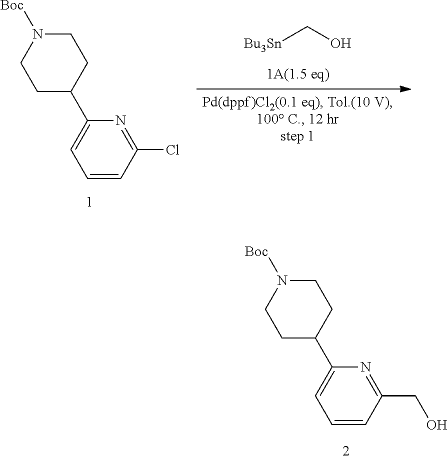

A mixture of 1 (2 g, 6.74 mmol, 1 eq), Pd(dppf)Cl₂ (493.08 mg, 673.87 μmol, 0.1 eq), 1A (3.25 g, 10.11 mmol, 1.5 eq) in Tol. (20 mL) was degassed and purged with N₂ for 3 times at 20° C., and then the mixture was stirred at 100° C. for 12 hr under N₂ atmosphere. LCMS (product: RT=1.061 min) showed compound 1 was consumed completely. The reaction mixture was cooled down to 20° C. and quenched by addition H₂O 20 mL at 20° C., and then extracted with ethyl acetate 90 mL (30 mL*3). The combined organic layers were washed with brine 40 mL (20 mL*2), dried over Na₂SO₄, filtered, and concentrated under reduced pressure to give a residue. The residue was purified by column chromatography (SiO₂, Petroleum ether/Ethyl acetate=5/1 to 1/1). 2 (440 mg, 1.50 mmol, 22.33% yield) was obtained as a red oil.

¹H NMR (400 MHz, CHLOROFORM-d) δ=7.65 (t, J=7.6 Hz, 1H), 7.08 (dd, J=3.6, 7.6 Hz, 2H), 4.75 (s, 2H), 4.35-4.21 (m, 2H), 2.98-2.76 (m, 3H), 1.93 (d, J=13.2 Hz, 2H), 1.75 (dq, J=4.4, 12.6 Hz, 3H), 1.49 (s, 9H)

### General Procedure for Preparation of Intermediate 3:

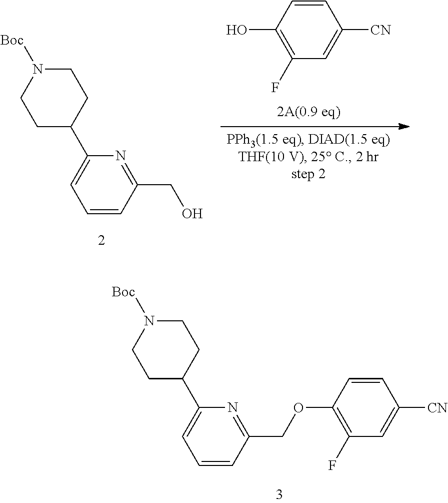

A mixture of 2 (200 mg, 684.06 μmol, 1 eq), 2A (84.41 mg, 615.65 μmol, 0.9 eq), PPh₃ (269.13 mg, 1.03 mmol, 1.5 eq), DIAD (207.48 mg, 1.03 mmol, 198.93 μL, 1.5 eq) in THF (2 mL) was degassed and purged with N₂ for 3 times, and then the mixture was stirred at 25° C. for 2 hr under N₂ atmosphere. LCMS (product: RT=1.429 min) showed the 2 was consumed completely. The reaction mixture was quenched by addition H₂O 5 mL at 20° C. and extracted with ethyl acetate 15 mL (5 mL*3). The combined organic layers were washed with brine 10 mL (5 mL*2), dried over Na₂SO₄, filtered, and concentrated under reduced pressure to give a residue. The residue was purified by column chromatography (SiO₂, Petroleum ether/Ethyl acetate=1/1 to 0/1). 3 (230 mg, crude) was obtained as a yellow oil.

### General Procedure for Preparation of Intermediate 4:

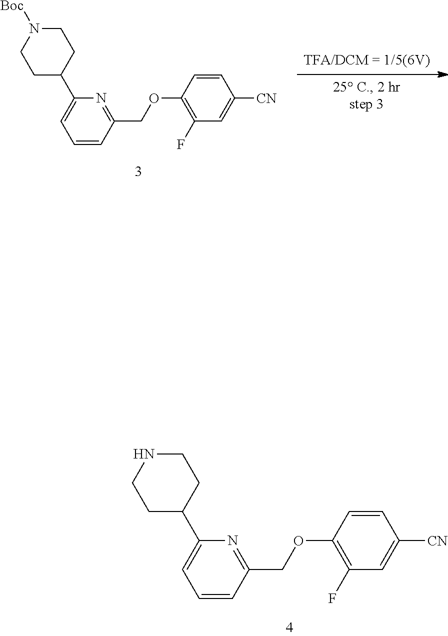

To a solution of 3 (100 mg, 243.03 μmol, 1 eq) in DCM (0.5 mL) TFA (0.1 mL) was added. The mixture was stirred at 25° C. for 2 hr. LCMS (product: RT=0.337 min) showed the 3 was consumed completely. The reaction mixture was added to sat. NaHCO₃ to adjust pH=7-8. The reaction was extracted with ethyl acetate 15 mL (5 mL*3). The combined organic layers were washed with brine 10 mL (5 mL*2), dried over Na₂SO₄, filtered, and concentrated under reduced pressure to give a residue. 4 (170 mg, crude) was obtained as yellow oil.

### General Procedure for Preparation of Intermediate 5:

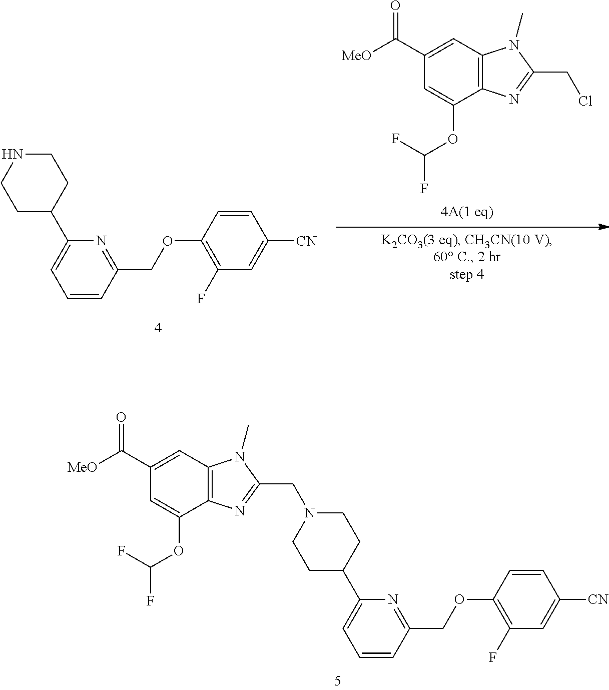 [SMILES: N#Cc1ccc(OCc2cccc(C3CCNCC3)n2)c(F)c1 ; COC(=O)c1cc(OC(F)F)c2nc(CCl)n(C)c2c1 ; "4A(1 eq)" ; "K2CO3(3 eq), CH3CN(10 V),60° C., 2 hrstep 4" ; COC(=O)c1cc(OC(F)F)c2nc(CN3CCC(c4cccc(COc5ccc(C#N)cc5F)n4)CC3)n(C)c2c1]

A mixture of 4 (143.07 mg, 459.50 μmol, 2 eq), 4A (70 mg, 229.75 μmol, 1 eq), K₂CO₃ (95.26 mg, 689.26 μmol, 3 eq), in CH₃CN (1 mL) was degassed and purged with N₂ for 3 times, and then the mixture was stirred at 60° C. for 2 hr under N₂ atmosphere. LCMS (product: RT=0.434 min) showed 4 and 4A was consumed completely. The reaction mixture was quenched by addition H₂O 2 mL at 20° C. and extracted with ethyl acetate 9 mL (3 mL*3). The combined organic layers were washed with brine 10 mL (5 mL*2), dried over Na₂SO₄, filtered, and concentrated under reduced pressure to give a residue. The residue was purified by column chromatography (SiO₂, Petroleum ether/Ethyl acetate=1/1 to 0/1). 5 (90 mg, 155.29 μmol, 67.59% yield) was obtained as a white solid.

LCMS: RT=0.430 min, MS cal.: 579.2, [M+H]⁺=580.2

¹H NMR (400 MHz, METHANOL-d4) δ=8.12 (s, 1H), 7.77 (t, J=8.0 Hz, 1H), 7.67 (s, 1H), 7.56 (dd, J=2.0, 10.8 Hz, 1H), 7.50 (d, J=8.8 Hz, 1H), 7.39 (d, J=8.0 Hz, 1H), 7.33 (t, J=8.4 Hz, 1H), 7.28-7.24 (m, 1H), 7.26 (t, J=72.4 Hz, 1H), 5.30 (s, 2H), 4.03 (s, 3H), 3.96 (s, 3H), 3.94 (s, 2H), 3.06 (d, J=11.6 Hz, 2H), 2.83-2.71 (m, 1H), 2.43-2.30 (m, 2H), 1.96-1.82 (m, 4H)

### General Procedure for Preparation of Compound 130 (6):

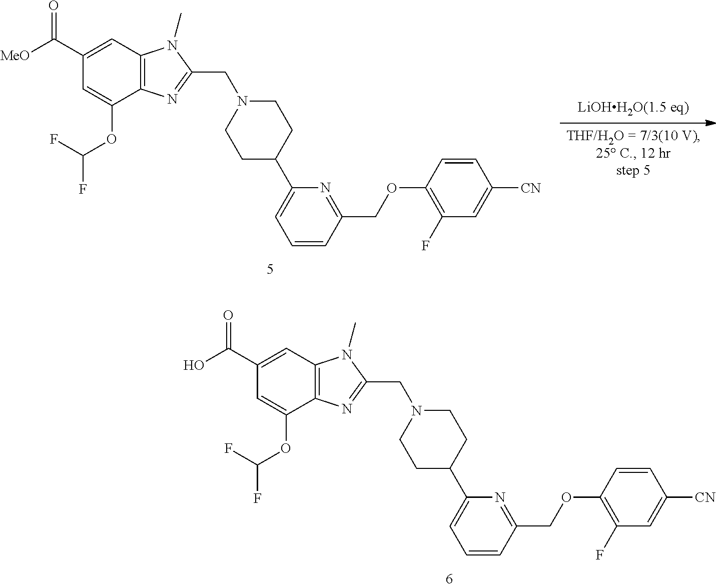

To a solution of 5 (90 mg, 155.29 μmol, 1 eq) in THF (0.7 mL) was added LiOH·H₂O (9.77 mg, 232.93 μmol, 1.5 eq) in H₂O (0.3 mL). The mixture was stirred at 25° C. for 12 hr under N₂ atmosphere. TLC (PE/EtOAc=2/1, compound 5 Rr-0.42) showed the 5 was consumed completely. The reaction mixture was filtered. The reaction was purified by prep-HPLC (column: Waters Xbridge BEH C18 100*30 mm*10 um; mobile phase: [H₂O (10 mM NH₄HCO₃)—CH₃CN]; gradient: 20%-50% B over 8.0 min). 2-((4-(6-((4-Cyano-2-fluorophenoxy)methyl)pyridin-2-yl)piperidin-1-yl)methyl)-4-(difluoromethoxy)-1-methyl-1H-benzo[d]imidazole-6-carboxylic acid (14.81 mg, 25.43 μmol, 16.37% yield, 97.10% purity) was obtained as a white solid.

LCMS: RT=2.510 min, MS cal.: 565.2, [M+H]⁺=566.2

HPLC: RT=9.698 min.

¹H NMR (400 MHz, METHANOL-d4) δ=8.11 (s, 1H), 7.78 (t, J=8.0 Hz, 1H), 7.68 (s, 1H), 7.60-7.54 (m, 1H), 7.51 (d, J=8.8 Hz, 1H), 7.40 (d, J=8.0 Hz, 1H), 7.33 (t, J=8.6 Hz, 1H), 7.27 (d, J=8.0 Hz, 1H), 7.26 (t, J=74.4 Hz, 1H), 5.30 (s, 2H), 4.02 (s, 3H), 4.00 (s, 2H), 3.12 (d, J=11.2 Hz, 2H), 2.87-2.73 (m, 1H), 2.54-2.35 (m, 2H), 2.00-1.85 (m, 4H)

### Example A273—Preparation of Compound 89. 2-(((1S,5R,6S)-5-((S)-2-(5-Chloropyridin-2-yl)-2-methylbenzo[d][1,3]dioxol-4-yl)-2-azabicyclo[4.1.0]heptan-2-yl)methyl)-4-methoxy-1-(((S)-oxetan-2-yl)methyl)-1H-benzo[d]imidazole-6-carboxylic acid (Compound 89) (13)

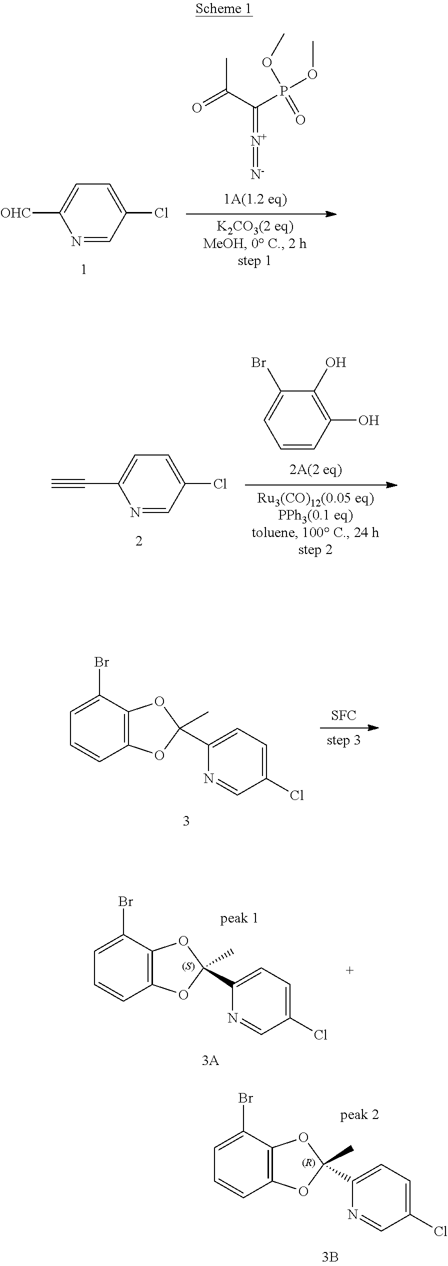

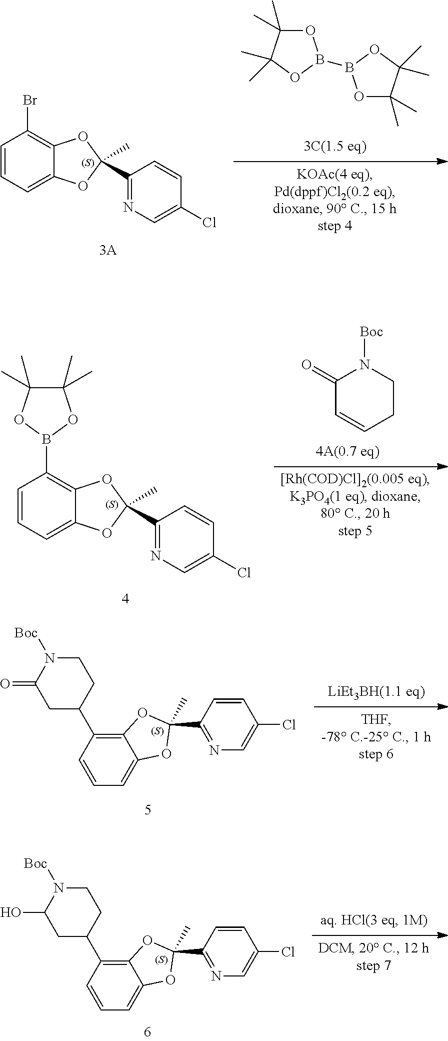

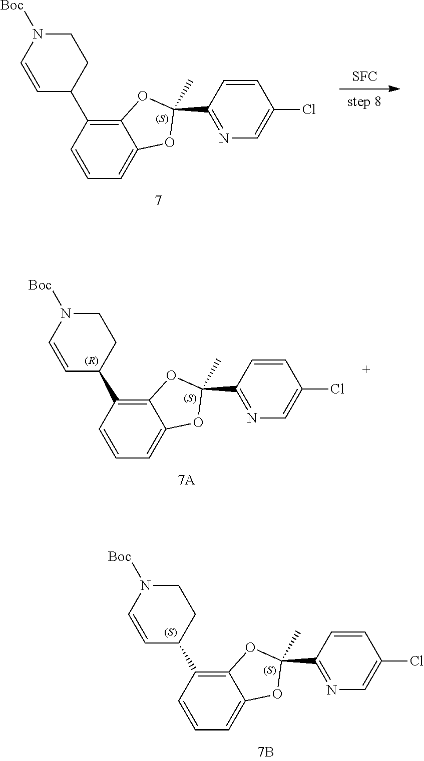

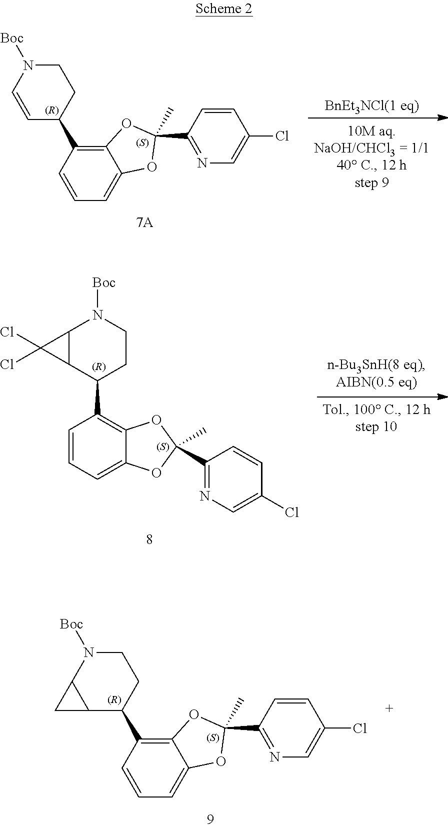

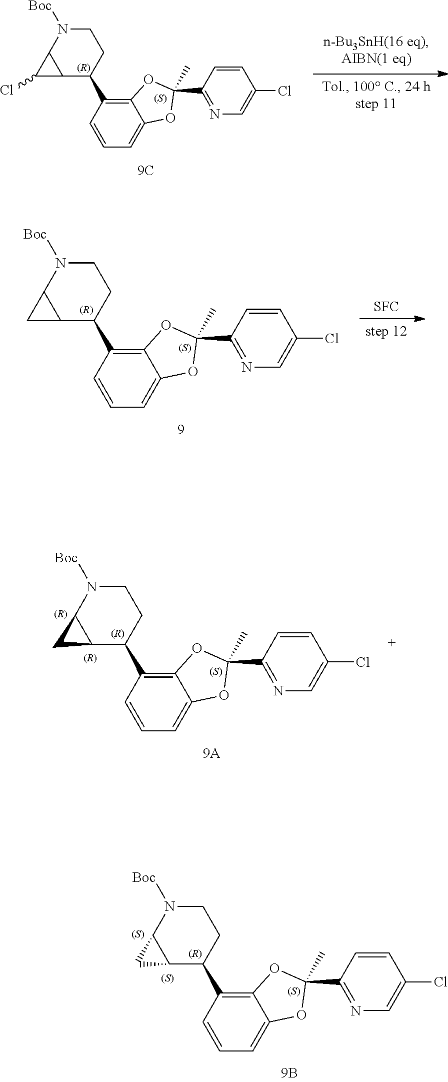

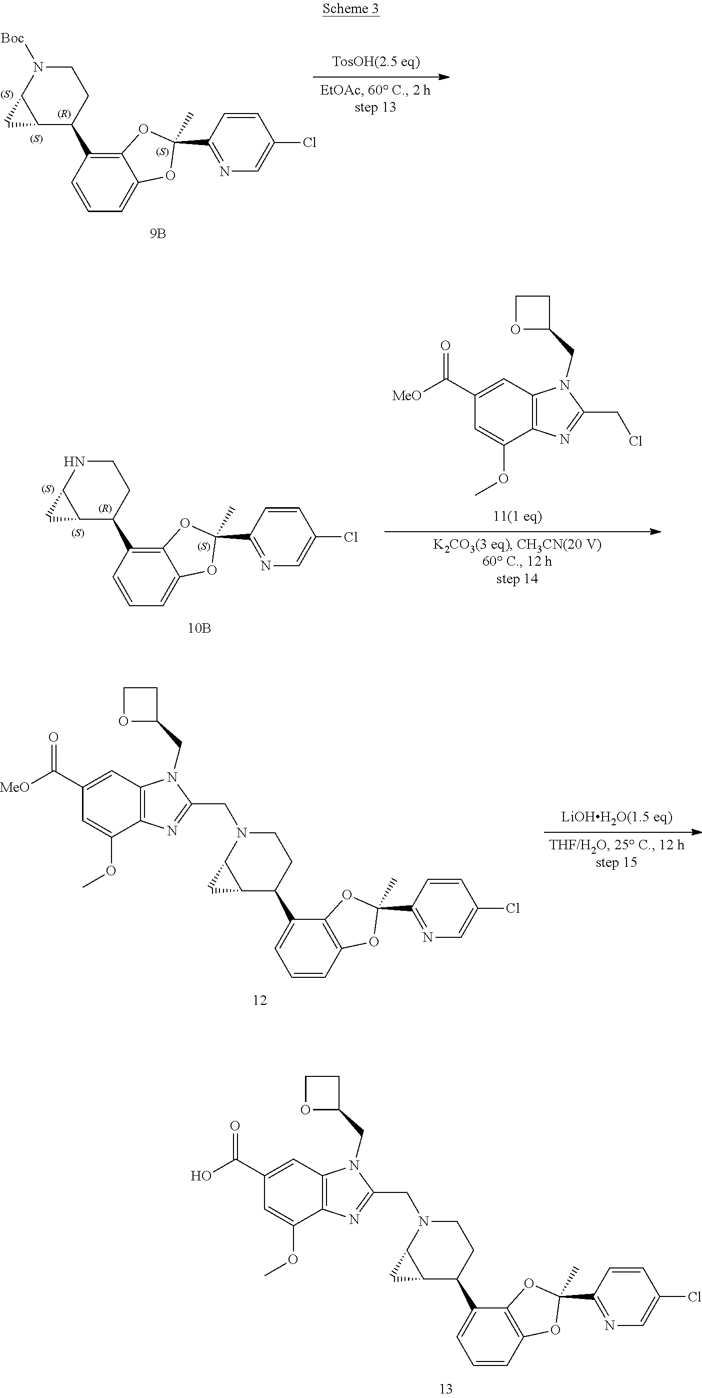

### General Procedure for Preparation of Intermediate 2:

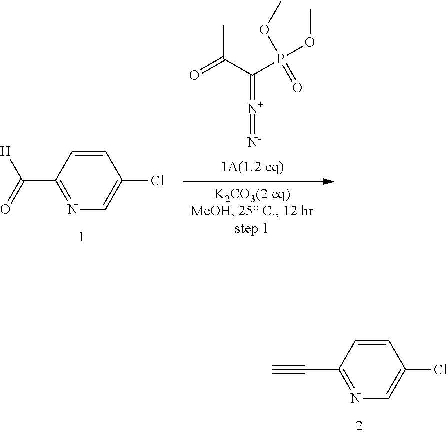

A mixture of 1A (48.86 g, 254.32 mmol, 1.2 eq), 1 (30 g, 211.93 mmol, 1 eq), K₂CO₃ (58.58 g, 423.86 mmol, 2 eq) in MeOH (300 mL) was degassed and purged with N₂ for 3 times, and then the mixture was stirred at 25° C. for 12 hr under N₂ atmosphere. TLC (Petroleum ether/Ethyl acetate=10/1, R_{f}=0.50) indicated 1 was consumed completely. The reaction mixture was poured into water (200 mL) and extracted with ethyl acetate (100 mL*2). The combined organic phase was concentrated in vacuo to give a residue. The residue was purified by column chromatography (SiO₂, Petroleum ether/Ethyl acetate=100/1 to 0/1). 2 (15 g, 109.04 mmol, 51.45% yield) was obtained as a white solid.

LCMS: RT=0.405 min, MS cal.: 137.57, [M+H]⁺=138.2

¹H NMR (400 MHz, CHLOROFORM-d) δ=8.52-8.40 (m, 1H) 7.60-7.52 (m, 1H) 7.38-7.30 (m, 1H) 3.16-3.12 (m, 1H)

### General Procedure for Preparation of Intermediate 3:

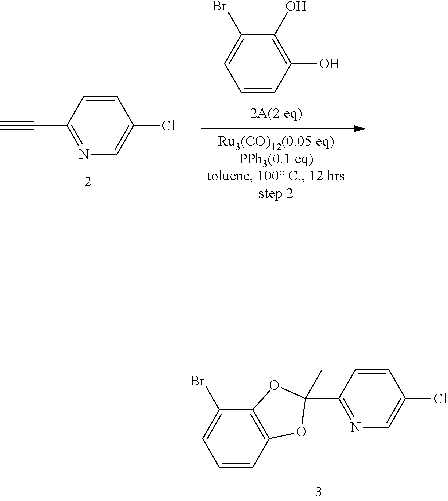

A mixture of 2 (15 g, 109.04 mmol, 1 eq), 2A (41.22 g, 218.08 mmol, 2 eq), PPh₃ (2.86 g, 10.90 mmol, 0.1 eq), Ru₃ (CO)₁₂ (3.49 g, 5.45 mmol, 0.05 eq) in Tol. (160 mL) was degassed and purged with N₂ for 3 times at 20° C., and then the mixture was stirred at 100° C. for 12 hr under N₂ atmosphere. TLC (Petroleum ether/Ethyl acetate=10/1 R_{f}=0.50) indicated 2 was consumed completely. The reaction mixture was poured into water (100 mL) and extracted with ethyl acetate (150 mL*2). The combined organic phase was concentrated in vacuo to give a residue. The residue was purified by column chromatography (SiO₂, Petroleum ether/Ethyl acetate=100/1 to 0/1). 3 (15 g, 45.93 mmol, 42.12% yield) was obtained as a white oil.

LCMS: RT=0.625 min, MS cal.: 326.57, [M+H]⁺=328.0

¹H NMR (400 MHz, CHLOROFORM-d) δ=8.61-8.53 (m, 1H) 7.69-7.61 (m, 1H) 7.58-7.50 (m, 1H) 6.90 (dd, J=8.0, 1.2 Hz, 1H) 6.73-6.59 (m, 2H) 2.01 (s, 3H)

### General Procedure for Preparation of Intermediate 3A & 3B:

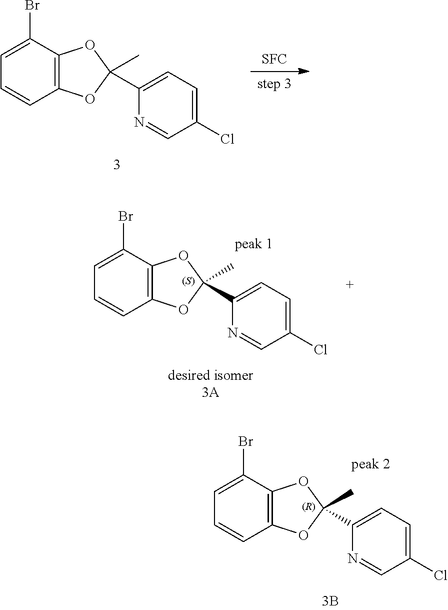

3 (100 g, 306.21 mmol, 1 eq) was purified by SFC (column: DAICEL CHIRALPAK AY (250 mm*50 mm, 10 um); mobile phase: [CO₂-EtOH (0.1% NH₃WATER)]; B %: 15%, isocratic elution mode) to give 3A (50 g, 153.11 mmol, 50.00% yield) was obtained as a white oil. 3B (50 g, 153.11 mmol, 50.00% yield) was obtained as a white oil.

3A & 3B SFC: peak 1 (3A) RT=0.624 min, purity: 49.64%;

peak 2 (3B) RT-0.698 min, purity: 50.36%

3B SFC: RT=0.691 min, purity: 98.74%

¹H NMR (400 MHz, CHLOROFORM-d) δ=8.56 (d, J=2.4 Hz, 1H), 7.64 (dd, J=2.4, 8.4 Hz, 1H), 7.54 (d, J=8.4 Hz, 1H), 6.90 (dd, J=1.2, 8.0 Hz, 1H), 6.72-6.60 (m, 2H), 2.04 (s, 3H)

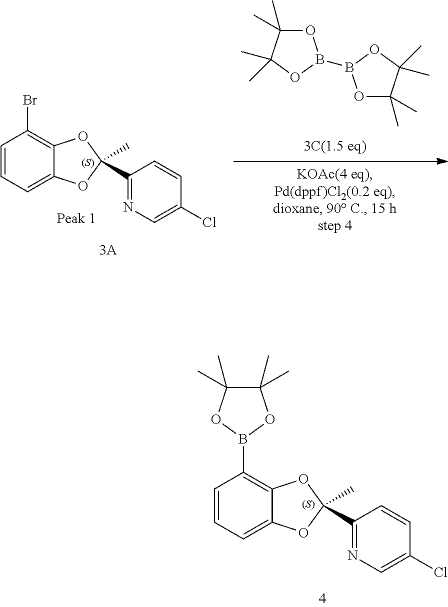

A mixture of 3A (10 g, 30.62 mmol, 1 eq), 3C (11.66 g, 45.93 mmol, 1.5 eq), KOAc (6.01 g, 61.24 mmol, 2 eq), Pd(dppf)Cl₂ (2.50 g, 3.06 mmol, 0.1 eq) in dioxane (200 mL) was degassed and purged with N₂ for 3 times, and then the mixture was stirred at 90° C. for 15 hr under N₂ atmosphere. The reaction was batched for 6. LC-MS (RT=2.204 min) showed 61% of desired compound was detected. TLC (Petroleum ether/Ethyl acetate=5/1, R_{f}=0.50) indicated 3A was consumed completely. 6 reactions were combined for workup. The reaction mixture was filtered first and then diluted with water 60 mL and extracted with ethyl acetate 2100 mL (700 mL*3). The combined organic layers were washed with brine (100 mL), dried over Na₂SO₄, filtered and concentrated under reduced pressure to give a residue. The residue was purified by column chromatography (SiO₂, Petroleum ether/Ethyl acetate=100/1 to 3/1). 4 (55 g, 147.20 mmol, 80.12% yield) was obtained as a white solid.

LCMS: RT=2.210 min, MS cal.: 373.13, [M+H]⁺=374.2

¹H NMR (400 MHz, CHLOROFORM-d) δ=8.62 (d, J=2.0 Hz, 1H), 7.71-7.62 (m, 2H), 7.20 (dd, J=1.2, 7.6 Hz, 1H), 6.91-6.86 (m, 1H), 6.83-6.78 (m, 1H), 2.10 (s, 3H), 1.37 (d, J=3.6 Hz, 12H)

### General Procedure for Preparation of Intermediate 5:

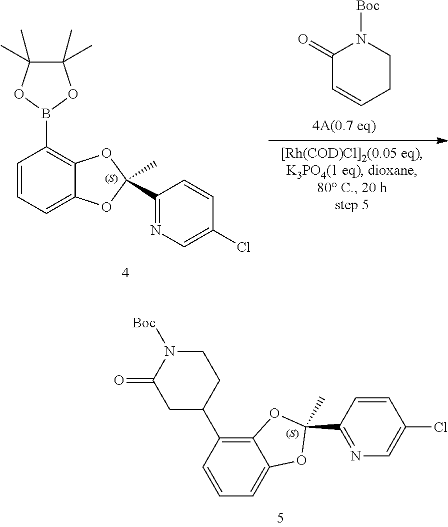

A mixture of 4 (15 g, 40.15 mmol, 1 eq), 4A (5.54 g, 28.10 mmol, 0.7 eq), [Rh(COD)Cl]₂ (989.76 mg, 2.01 mmol, 0.05 eq), K₃PO₃ (8.52 g, 40.15 mmol, 0.1 eq) in dioxane (150 mL) was degassed and purged with N₂ for 3 times, and then the mixture was stirred at 80° C. for 20 hr under N₂ atmosphere. LC-MS (RT=2.093 min) showed 75% of desired compound was detected. TLC (Petroleum ether/Ethyl acetate=5/1 R_{f}=0.30) indicated 4 was consumed completely. The reaction mixture was filtered first and then diluted with water 20 mL and extracted with ethyl acetate 450 mL (150 mL*3). The combined organic layers were washed with brine (30 mL), dried over Na₂SO₄, filtered and concentrated under reduced pressure to give a residue. The residue was purified by column chromatography (SiO₂, Petroleum ether/Ethyl acetate=100/1 to 1/1). 5 (13 g, 29.22 mmol, 72.78% yield) was obtained as a yellow oil.

LCMS: RT=2.076 min, MS cal.: 444.15, [M+H]⁺=445.1

¹H NMR (400 MHz, CHLOROFORM-d) δ=8.63 (t, J=2.0 Hz, 1H), 7.70 (dd, J=2.4, 8.4 Hz, 1H), 7.62-7.51 (m, 1H), 6.87-6.73 (m, 2H), 6.66 (d, J=7.6 Hz, 1H), 3.88 (tdd, J=4.6, 7.6, 12.4 Hz, 1H), 3.70-3.53 (m, 1H), 3.34-3.15 (m, 1H), 2.88-2.70 (m, 2H), 2.26-2.08 (m, 2H), 2.06-2.03 (m, 3H), 1.55 (d, J=1.6 Hz, 9H)

### General Procedure for Preparation of Intermediate 6:

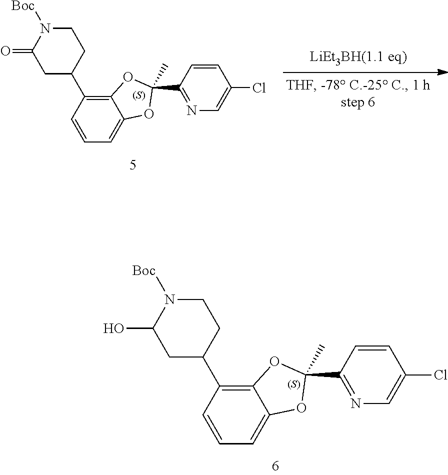

The mixture of 5 (13 g, 29.22 mmol, 1 eq) in THF (130 mL) was degassed and purged with N₂ for 3 times, and then the mixture was stirred at 25° C. for 0.5 hr under N₂ atmosphere. At −78° C. inner temperature, LiEt₃BH (1 M, 32.14 mL, 1.1 eq) was added dropwise to the reaction mixture. The mixture was stirred at −78° C. for 1 hr. The reaction was monitored by LCMS. LCMS showed 5 was consumed and desired Ms was detected. After 1 hr, water 200 mL was added to the reaction mixture at −78° C. within 10 min. The mixture was extracted with ethyl acetate (200 mL*3). The combined organic layers were washed with brine 100 mL, dried over Na₂SO₄, filtered and concentrated under reduced pressure to give a residue. The crude was used directly for next step. 6 (13 g, crude) was obtained as a colorless oil.

LCMS: RT=0.603 min, MS cal.: 446.2, [Mass]=329.3

### General Procedure for Preparation of Intermediate 7:

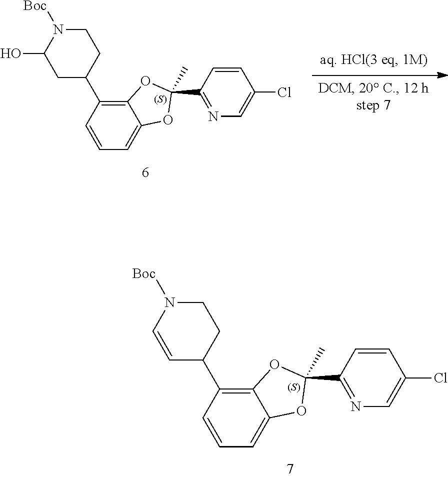

To a solution of 6 (13 g, 29.09 mmol, 1 eq) in DCM (130 mL) was added HCl (1 M, 87.26 mL, 3 eq). The mixture was stirred at 25° C. for 12 hr. LC-MS (ET43536-1283-p1a1) showed 6 was consumed completely and one main peak with desired mass was detected. The residue was diluted with Na₂CO₃ 50 mL and extracted with ethyl acetate 150 mL (50 mL*3). The combined organic layers were washed with water 60 mL, dried over Na₂SO₄, filtered, and concentrated under reduced pressure to give residue. The residue was purified by column chromatography (SiO₂, Petroleum ether/Ethyl acetate=100/1 to 10/1). 8 (6.5 g, 15.15 mmol, 52.10% yield) was obtained as a white solid.

LCMS: RT=0.710 min, MS cal.: 428.1, [M−100+H]⁺=329.2

1H NMR (400 MHz, CHLOROFORM-d) δ=8.67-8.59 (s, 1H), 7.70 (dd, J=2.4, 8.4 Hz, 1H), 7.60 (d, J=8.4 Hz, 1H), 7.09 (br d, J=7.2 Hz, 1H), 6.95 (d, J=7.6 Hz, 1H), 6.84-6.76 (m, 1H), 6.76-6.70 (m, 2H), 5.00-4.76 (m, 1H), 3.72 (1H), 3.67-3.48 (s, 2H), 2.22-2.11 (d, 1H), 2.10-2.01 (m, 5H), 1.93-1.75 (m, 1H), 1.61 (s, 1H), 1.52 (s, 11H), 1.27 (t, J=7.2 Hz, 1H)

### General Procedure for Preparation of Intermediate 7A & 7B:

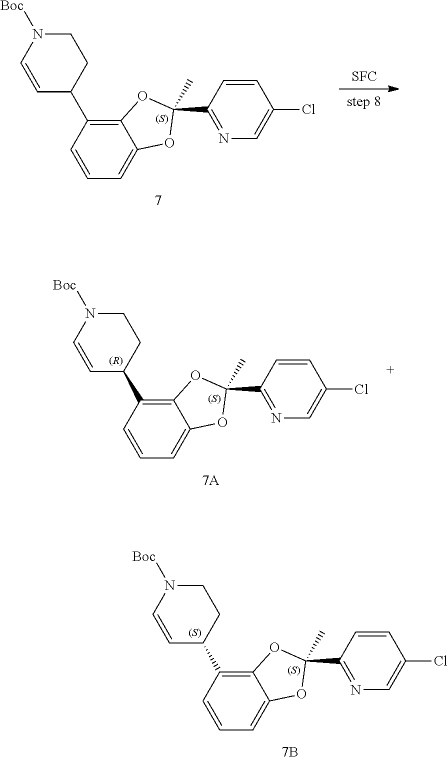

7 (10 g, 23.32 mmol, 1 eq) was purified by SFC (condition: column: DAICEL CHIRALPAK IG (250 mm*50 mm, 10 um); mobile phase: [CO₂-MeOH (0.1% NH₃WATER)]; B %: 40%, isocratic elution mode). 7A (5.4 g, 12.59 mmol, 54.00% yield) was obtained as a white solid. 7B (5 g, 11.66 mmol, 50.00% yield) was obtained as a white solid.

7A SFC: RT=1.246 min, purity: 100.00%

7B SFC: RT=1.826 min, purity: 100.00%

¹H NMR (400 MHz, CHLOROFORM-d) δ=8.63 (d, J=2.4 Hz, 1H), 7.71 (d, J=8.4 Hz, 1H), 7.60 (d, J=8.4 Hz, 1H), 7.09 (d, J=7.2 Hz, 1H), 6.95 (d, J=8.0 Hz, 1H), 6.83-6.62 (m, 3H), 5.00-4.78 (m, 1H), 3.73 (s, 1H), 3.67-3.45 (m, 2H), 2.20-2.03 (m, 5H), 1.90-1.69 (m, 1H), 1.52 (s, 11H)

¹H NMR (400 MHz, CHLOROFORM-d) δ=8.64 (d, J=2.4 Hz, 3H), 7.70 (dd, J=2.4, 8.4 Hz, 3H), 7.64-7.57 (m, 3H), 7.09 (d, J=7.2 Hz, 1H), 6.95 (d, J=8.0 Hz, 2H), 6.86-6.78 (m, 3H), 6.76-6.69 (m, 6H), 4.95-4.69 (m, 3H), 3.71 (d, J=2.4 Hz, 3H), 3.64-3.49 (m, 6H), 2.25-2.11 (m, 3H), 2.07 (s, 9H), 1.93-1.77 (m, 3H), 1.57 (s, 5H), 1.54-1.52 (m, 9H)

### General Procedure for Preparation of Intermediate 8:

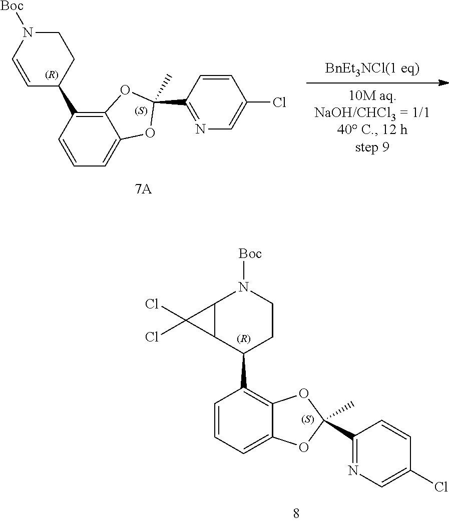

To a mixture of 7A (4.5 g, 10.49 mmol, 1 eq) in CHCl₃ (45 mL) was added BnEt₃NCl (2.39 g, 10.49 mmol, 1 eq). NaOH (10 M, 45 mL, 42.89 eq) was added dropwise to the reaction mixture. After the addition, the mixture was stirred at 40° C. for 12 hr. LCMS (ET53089-718-P1R2, product: RT=2.825 min) showed the 7A was consumed completely. The reaction mixture was added to 50 mL H₂O at 0° C. within 5 min. The mixture was extracted by ethyl acetate (100 mL*3). Then the organic phase was combined and washed by H₂O (50 mL), aq. NaCl (50 mL), dried with Na₂SO₄, filtered. The organic phase was concentrated under reduced pressure at 40° C. to give a residue. The residue was purified by column chromatography (SiO₂, Petroleum ether/Ethyl acetate=100/1 to 10/1). 8 (2.6 g, 5.08 mmol, 48.42% yield) was obtained as a white solid.

LCMS: RT=2.831 min, MS cal.: 511.8, [M−55]⁺=455.0

¹H NMR (400 MHz, CHLOROFORM-d) δ=8.62 (d, J=2.0 Hz, 1H), 7.71 (dd, J=2.4, 8.4 Hz, 1H), 7.59 (d, J=8.4 Hz, 1H), 6.87-6.71 (m, 3H), 3.43-3.34 (m, 2H), 3.23 (dt, J=4.4, 11.7 Hz, 1H), 2.93-2.84 (m, 1H), 2.35-2.23 (m, 1H), 2.08 (s, 3H), 1.98 (qd, J=4.4, 13.6 Hz, 1H), 1.86-1.71 (m, 1H), 1.61-1.54 (m, 9H)

### General Procedure for Preparation of Intermediate 9 & 9C:

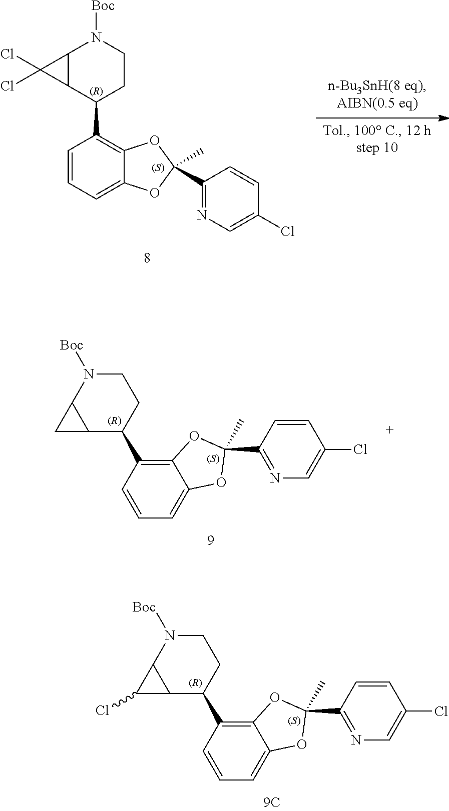

To a mixture of 8 (2.4 g, 4.69 mmol, 1 eq) in Tol. (25 mL) was added AIBN (384.99 mg, 2.34 mmol, 0.5 eq) and n-Bu₃SnH (10.92 g, 37.51 mmol, 9.94 mL, 8 eq) at 0° C. After the addition, the mixture was stirred at 100° C. for 12 hr. LCMS (product: RT=2.652 min) showed 8 was consumed completely. The reaction mixture was added to 15 mL (KF) at 0° C. The mixture was extracted by ethyl acetate (50 mL*3). Then organic phase was combined and washed by H₂O (30 mL), aq. NaCl (30 mL), dried with Na₂SO₄, filtered. The organic phase was concentrated under reduced pressure at 40° C. to give a residue. The residue was purified by column chromatography (SiO₂, Petroleum ether/Ethyl acetate=100/1 to 10/1). Intermediate 9 & 9C (1.36 g, 3.07 mmol, 65.48% yield) was obtained as a white solid. Intermediate 9 & 9C was a mixture. It was used to the next step directly.

LCMS: RT=2.652 min, MS cal.: 442.9, [M+H]⁺=443.1

### General Procedure for Preparation of Intermediate 9:

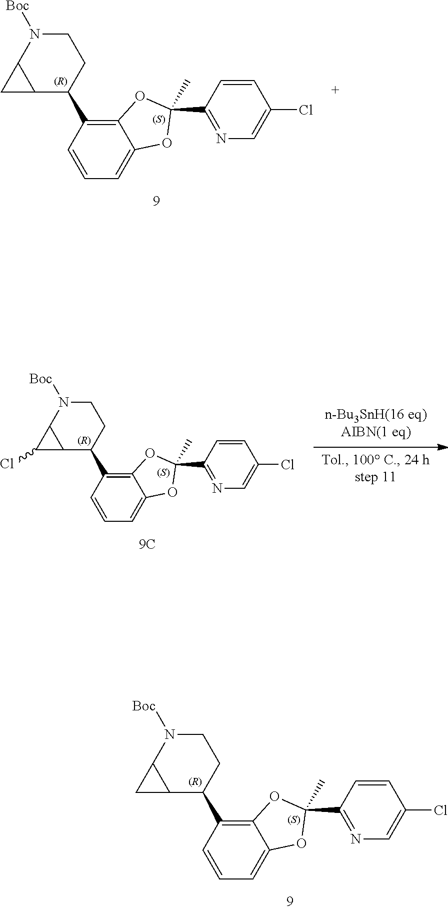

To a mixture of 9 & 9C (1.16 g, 2.27 mmol, 1 eq) in Tol. (15 mL) was added AIBN (186.08 mg, 1.13 mmol, 0.5 eq) and n-Bu₃SnH (5.28 g, 18.13 mmol, 4.81 mL, 8 eq). After the addition, the mixture was stirred at 100° C. for 12 hr. AIBN (186.08 mg, 1.13 mmol, 0.5 eq) and n-Bu₃SnH (5.28 g, 18.13 mmol, 4.81 mL, 8 eq) were added dropwise to the reaction mixture. After the addition, the mixture was stirred at 100° C. for 12 hr. LCMS (product: RT=7.243 min) showed the 9 & 9C was consumed completely. The reaction mixture was added to 30 mL (KF) at 0° C. The mixture was extracted by ethyl acetate (30 mL*3). Then organic phase was combined and washed by H₂O (15 mL), aq. NaCl (15 mL), dried with Na₂SO₄, filtered. The organic phase was concentrated under reduced pressure at 40° C. to give a residue. The residue was purified by column chromatography (SiO₂, Petroleum ether/Ethyl acetate=100/1 to 10/1). 9 (860 mg, 1.94 mmol, 85.67% yield) was obtained as a white solid.

LCMS: RT=7.189 min, MS cal.: 442.9, [M+H]⁺=443.1

HPLC: RT=2.133 min, purity=94.106%

¹H NMR (400 MHz, CHLOROFORM-d) δ=8.62 (d, J=2.0 Hz, 1H), 7.70 (br d, J=8.4 Hz, 1H), 7.57 (d, J=8.4 Hz, 1H), 6.94 (br d, J=7.2 Hz, 1H), 6.89-6.74 (m, 2H), 3.55-3.25 (m, 2H), 3.15-2.80 (m, 2H), 2.06 (s, 3H), 1.82-1.58 (m, 2H), 1.54-1.45 (m, 9H), 1.39-1.25 (m, 2H), 1.04-0.81 (m, 3H), 0.47-0.33 (m, 1H)

### General Procedure for Preparation of Intermediate 9B:

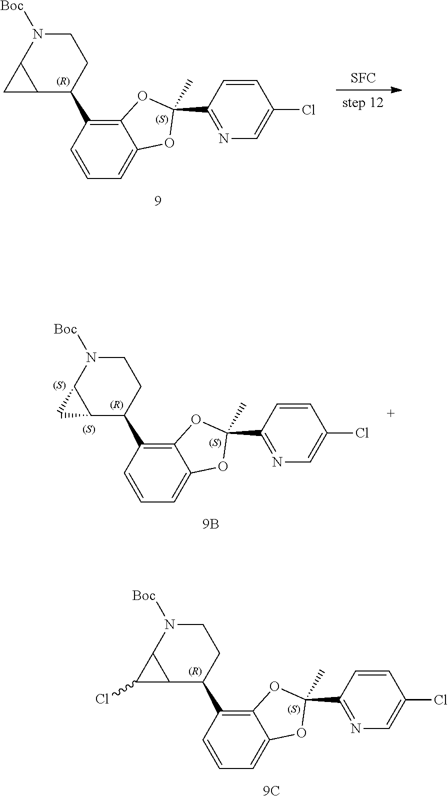

9 was purified by SFC (column: DAICEL CHIRALPAK AD (250 mm*30 mm, 10 um); mobile phase: [CO₂-IPA (0.1% NH₃H₂O)]; B %: 23%, isocratic elution mode). 9B (600 mg, 1.35 mmol, 61.86% yield) was obtained as a white solid. 9C (130 mg, 272.32 μmol, 12.44% yield) was obtained as a white solid.

LCMS: RT=7.173 min, MS cal.: 442.9, [M+H]⁺=443.1

HPLC: RT=2.215 min, purity=97.257%

SFC: ee=100%

¹H NMR (400 MHz, CHLOROFORM-d) δ=8.62 (d, J=2.0 Hz, 1H), 7.70 (dd, J=2.0, 8.4 Hz, 1H), 7.57 (d, J=8.4 Hz, 1H), 6.94 (d, J=7.2 Hz, 1H), 6.83 (t, J=7.6 Hz, 1H), 6.79-6.72 (m, 1H), 3.57-3.43 (m, 1H), 3.40-3.26 (m, 1H), 3.13-2.95 (m, 1H), 2.93-2.81 (m, 1H), 2.06 (s, 3H), 1.75 (ddd, J=4.4, 9.6, 13.6 Hz, 1H), 1.70-1.61 (m, 1H), 1.54-1.45 (m, 9H), 1.44-1.33 (m, 1H), 1.05-0.86 (m, 1H), 0.50-0.32 (m, 1H)

### General Procedure for Preparation of Intermediate 10B:

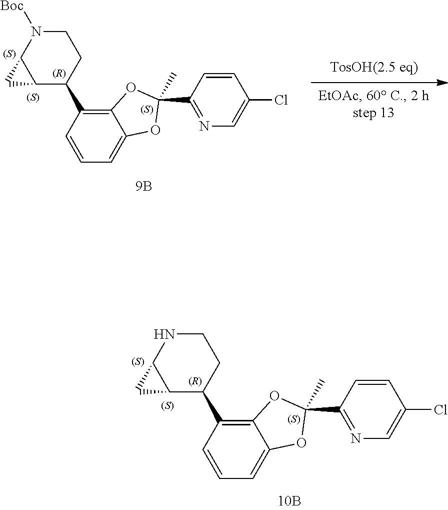

A mixture of 9B (400 mg, 903.07 μmol, 1 eq) and TosOH (388.78 mg, 2.26 mmol, 2.5 eq) in ethyl acetate (6 mL) was degassed and purged with N₂ for 3 times, and then the mixture was stirred at 60° C. for 2 hr under N₂ atmosphere. LCMS (product: RT=1.664 min) showed the 9B was consumed completely. The pH was adjusted to around 9 by progressively adding aq. NaHCO₃. The reaction mixture was diluted with H₂O 8 mL and extracted with ethyl acetate (10 mL*3). The combined organic layers were washed with brine 10 mL, dried over Na₂SO₄, filtered, and concentrated under reduced pressure to give a residue. 10B (410 mg, crude) was obtained as a yellow solid.

LCMS: RT=1.260 min, MS cal.: 342.8, [M+H]⁺=343.1

HPLC: RT=2.420 min, purity=93.087%

¹H NMR (400 MHz, CHLOROFORM-d) δ=8.62 (d, J=2.0 Hz, 1H), 7.72-7.65 (m, 1H), 7.58 (d, J=8.4 Hz, 1H), 7.01 (d, J=7.6 Hz, 1H), 6.84 (t, J=7.6 Hz, 1H), 6.74 (dd, J=1.2, 7.6 Hz, 1H), 5.31 (s, 1H), 3.47-3.34 (m, 1H), 2.80-2.71 (m, 1H), 2.67 (dt, J=4.0, 7.2 Hz, 1H), 2.56 (ddd, J=3.2, 6.8, 12.8 Hz, 1H), 2.07-2.05 (m, 3H), 1.75-1.64 (m, 1H), 1.50 (dtd, J=2.8, 6.8, 13.2 Hz, 1H), 1.17-1.07 (m, 1H), 0.79 (ddd, J=5.6, 6.8, 9.6 Hz, 1H), 0.57-0.48 (m, 1H)

### General Procedure for Preparation of Intermediate 12:

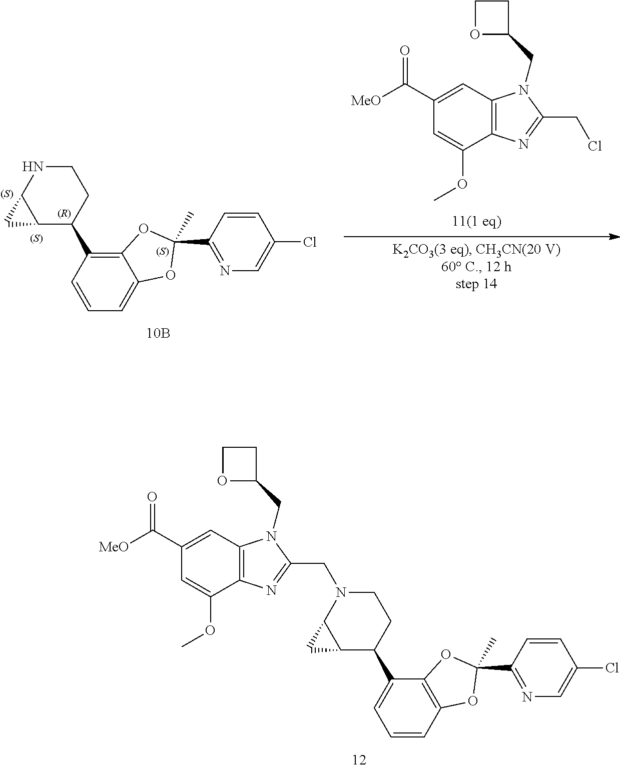

A mixture of 10B (79.17 mg, 230.94 μmol, 1.5 eq), 11 (50 mg, 153.96 μmol, 1 eq), K₂CO₃ (63.83 mg, 461.88 μmol, 3 eq) in CH₃CN (2 mL) was degassed and purged with N₂ for 3 times, and then the mixture was stirred at 60° C. for 12 hr under N₂ atmosphere. LCMS (Rt=0.456) showed 75% of desired compound was detected. The reaction mixture was diluted with H₂O 10 mL and extracted with ethyl acetate 30 mL (10 mL*3), dried over Na₂SO₄, filtered, and concentrated under reduced pressure to give a residue. The residue was purified by column chromatography (SiO₂, Petroleum ether/Ethyl acetate=100/1 to 1/1). 12 (60 mg, 95.07 μmol, 61.75% yield) was obtained as a white solid.

LCMS: RT=0.456 min, MS cal.: 630.2, [M+H]⁺=631.2/633.2

### General Procedure for Preparation of Compound 89 (13):

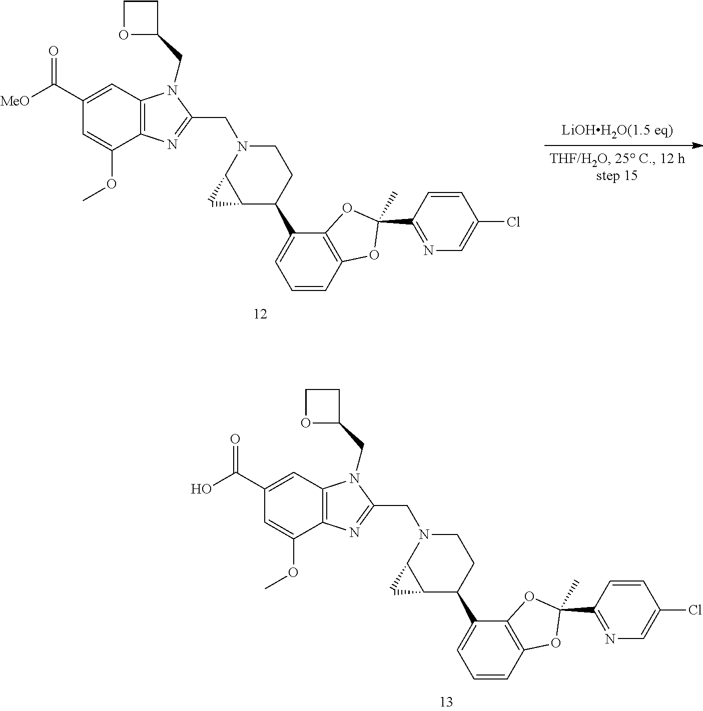 [SMILES: COC(=O)c1cc(OC)c2nc(CN3CC[C@@H](c4cccc5c4O[C@@](C)(c4ccc(Cl)cn4)O5)[C@@H]4C[C@@H]43)n(C[C@@H]3CCO3)c2c1 ; "LiOH·H2O(1.5 eq)" ; "THF/H2O, 25° C., 12 hstep 15" ; COc1cc(C(=O)O)cc2c1nc(CN1CC[C@@H](c3cccc4c3O[C@@](C)(c3ccc(Cl)cn3)O4)[C@@H]3C[C@@H]31)n2C[C@@H]1CCO1]

A mixture of 12 (55 mg, 87.15 μmol, 1 eq), LiOH·H₂O (5.49 mg, 130.72 μmol, 1.5 eq) in THF (0.7 mL) and H₂O (0.3 mL) was degassed and purged with N₂ for 3 times, and then the mixture was stirred at 25° C. for 12 hr under N₂ atmosphere. LCMS (Rt=1.691) showed 94.6% of desired compound was detected. The reaction was concentrated. The residue was purified by prep-HPLC (neutral condition column: Waters Xbridge Prep OBD C18 150*40 mm*10 um; mobile phase: [H₂O (10 mM NH₄HCO₃)-ACN]; gradient: 20%-70% B over 8.0 min). 2-(((1S,5R,6S)-5-((S)-2-(5-Chloropyridin-2-yl)-2-methylbenzo[d][1,3]dioxol-4-yl)-2-azabicyclo[4.1.0]heptan-2-yl)methyl)-4-methoxy-1-(((S)-oxetan-2-yl)methyl)-1H-benzo[d]imidazole-6-carboxylic acid (28.56 mg, 46.28 μmol, 53.11% yield, 98.74% purity) was obtained as a white solid.

LCMS: RT=2.600 min, MS cal.: 616.2, [M+H]⁺=617.2/619.2

HPLC: RT=10.636 min, purity: 98.74%

¹H NMR (400 MHz, METHANOL-d4) δ=8.58 (d, J=2.4 Hz, 1H), 7.94 (d, J=1.2 Hz, 1H), 7.86 (dd, J=2.4, 8.8 Hz, 1H), 7.64 (d, J=8.4 Hz, 1H), 7.44 (s, 1H), 6.87-6.79 (m, 2H), 6.71 (dd, J=1.6, 7.2 Hz, 1H), 5.25 (dq, J=2.4, 7.2 Hz, 1H), 4.91 (d, J=7.2 Hz, 1H), 4.73-4.57 (m, 2H), 4.47 (td, J=6.0, 9.2 Hz, 1H), 4.24 (d, J=13.6 Hz, 1H), 4.05-3.99 (m, 4H), 2.95 (ddd, J=3.2, 6.4, 9.6 Hz, 1H), 2.85-2.75 (m, 1H), 2.64-2.43 (m, 3H), 2.37-2.29 (m, 1H), 2.03 (s, 3H), 1.76-1.66 (m, 2H), 1.19 (ddt, J=3.2, 5.6, 8.8 Hz, 1H), 0.60-0.48 (m, 2H)

### Example A274—Preparation of Compound 104. 2-((4-(6-((4-Cyano-2-fluorobenzyl)oxy)pyridin-2-yl)piperidin-1-yl)methyl)-3,4-dihydro-5-oxa-1,2a-diazaacenaphthylene-6-carboxylic acid (Compound 104) (6)

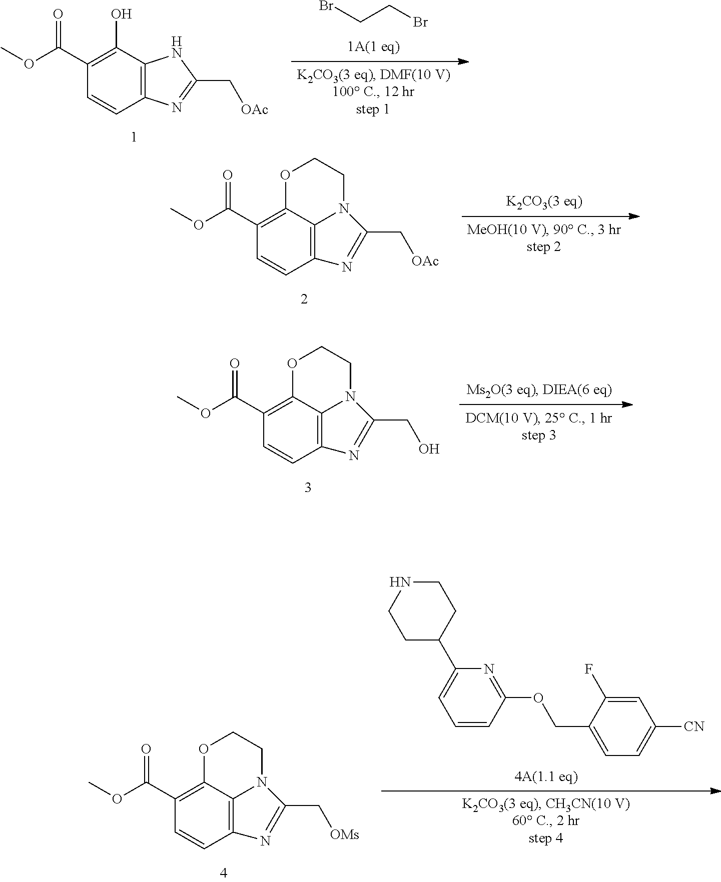

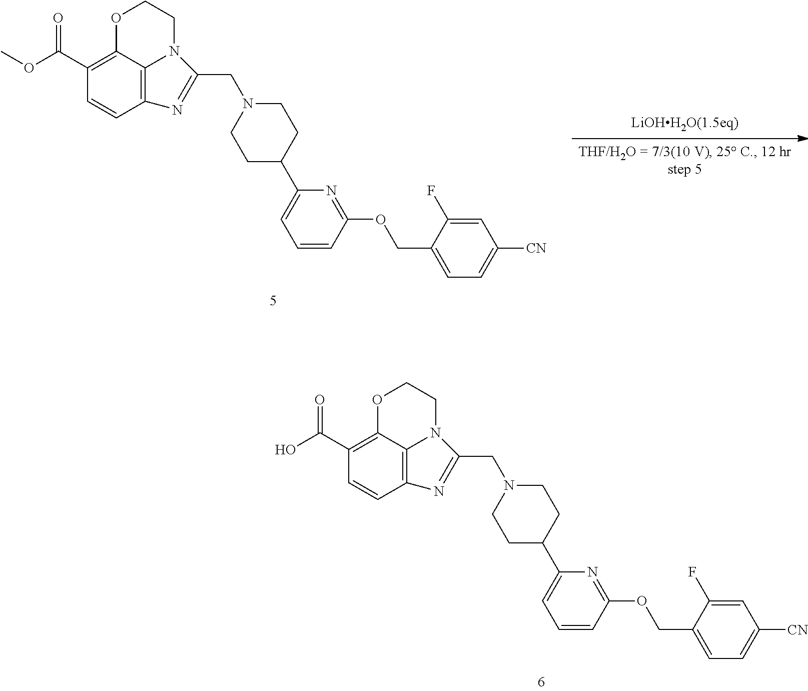

### General Procedure for Preparation of Intermediate 2:

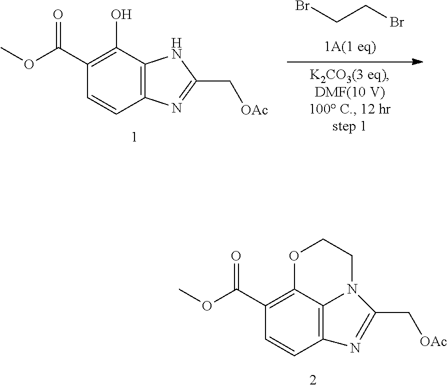

To a solution of 1 (600 mg, 2.27 mmol, 1 eq) in DMF (6 mL) was added K₂CO₃ (941.48 mg, 6.81 mmol, 3 eq) and 1A (426.58 mg, 2.27 mmol, 171.32 μL, 1 eq). The mixture was stirred at 100° C. for 12 hr. LCMS (RT=0.836 min) showed 1 was consumed completely and one main peak with desired mass was detected. The reaction mixture was filtered, the cake diluted by DCM (100 mL) and concentrated under reduced pressure to give a residue. 2 (650 mg, crude) was obtained as a brown solid.

### General Procedure for Preparation of Intermediate 3:

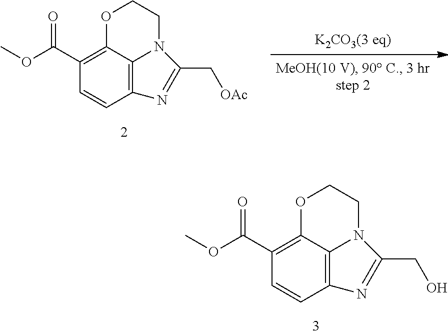 [SMILES: COC(=O)c1ccc2nc(COC(C)=O)n3c2c1OCC3 ; "K2CO3(3 eq)" ; "MeOH(10 V), 90° C., 3 hrstep 2" ; COC(=O)c1ccc2nc(CO)n3c2c1OCC3]

A mixture of 2 (0.65 g, 2.24 mmol, 1 eq), K₂CO₃ (928.48 mg, 6.72 mmol, 3 eq) in MeOH (7 mL) was degassed and purged with N₂ for 3 times, and then the mixture was stirred at 90° C. for 3 hr under N₂ atmosphere. LCMS (RT=0.695 min) showed 2 was consumed completely. The reaction mixture was diluted with H₂O (20 mL) and extracted with EtOAc (20 mL*5). The combined organic layers were dried over Na₂SO₄, filtered and concentrated under reduced pressure to give a residue. 3 (0.2 g, crude) was obtained as a yellow solid.

¹H NMR (400 MHz, DMSO-d₆) δ=7.60 (d, J=8.4 Hz, 1H), 7.19 (d, J=8.8 Hz, 1H), 4.76 (s, 2H), 4.60-4.54 (m, 2H), 4.52-4.45 (m, 2H), 3.81 (s, 3H)

### General Procedure for Preparation of Intermediate 4:

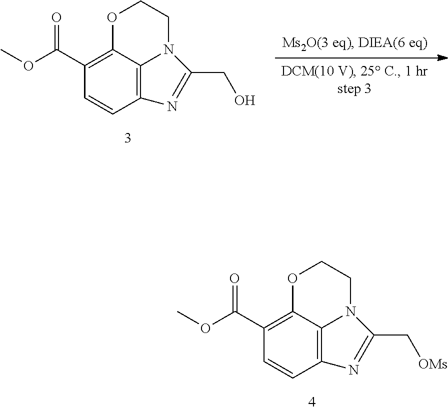 [SMILES: COC(=O)c1ccc2nc(CO)n3c2c1OCC3 ; "DCM(10 V), 25° C., 1 hrstep 3" ; "Ms2O(3 eq), DIEA(6 eq)" ; COC(=O)c1ccc2nc(COS(C)(=O)=O)n3c2c1OCC3]

A mixture of 3 (100 mg, 402.85 μmol, 1 eq), Ms₂O (210.52 mg, 1.21 mmol, 3 eq), DIEA (312.39 mg, 2.42 mmol, 421.01 μL, 6 eq) in DCM (1 mL) was degassed and purged with N₂ for 3 times, and then the mixture was stirred at 25° C. for 1 hr under N₂ atmosphere. LCMS (RT=0.318 min) showed 3 was consumed completely and one main peak with desired mass was detected. The reaction mixture was added HCOOH at 25° C. until pH=7-8, and then diluted with H₂O (7 mL) and extracted with DCM (7 mL*5). The combined organic layers were dried over Na₂SO₄, filtered and concentrated under reduced pressure to give a residue. 4 (130 mg, crude) was obtained as an orange oil.

### General Procedure for Preparation of Intermediate 5:

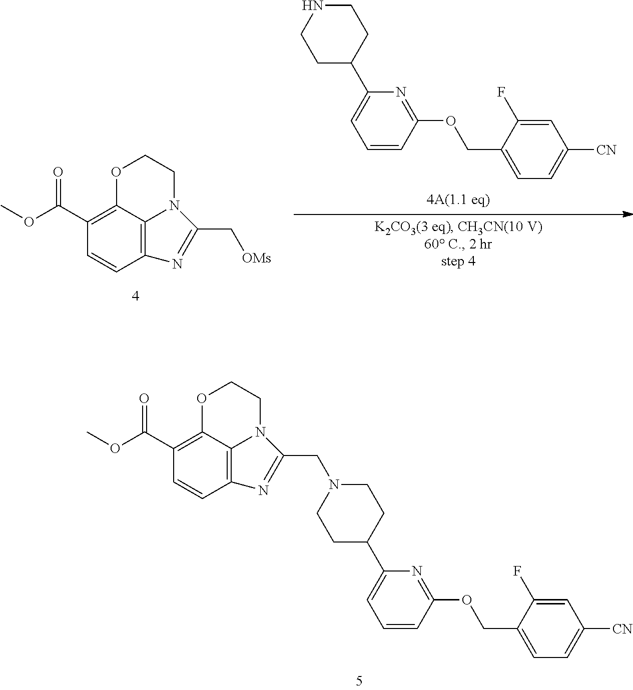 [SMILES: COC(=O)c1ccc2nc(COS(C)(=O)=O)n3c2c1OCC3 ; N#Cc1ccc(COc2cccc(C3CCNCC3)n2)c(F)c1 ; "4A(1.1 eq)" ; "K2CO3(3 eq), CH3CN(10 V)60° C., 2 hrstep 4" ; COC(=O)c1ccc2nc(CN3CCC(c4cccc(OCc5ccc(C#N)cc5F)n4)CC3)n3c2c1OCC3]

A mixture of 4 (130 mg, 398.38 μmol, 1 eq), 4A (136.44 mg, 438.21 μmol, 1.1 eq), K₂CO₃ (165.17 mg, 1.20 mmol, 3 eq) in ACN (2 mL) was degassed and purged with N₂ for 3 times, and then the mixture was stirred at 60° C. for 2 hr under N₂ atmosphere. TLC (Petroleum ether/Ethyl acetate=0/1, R_{f}=0.2) indicated 4 was consumed completely. The reaction mixture was diluted with H₂O (10 mL) and extracted with DCM (10 mL*5). The combined organic layers were dried over Na₂SO₄, filtered, and concentrated under reduced pressure to give a residue. The residue was purified by column chromatography (SiO₂, Petroleum ether/Ethyl acetate=100/1 to Ethyl acetate/Methanol=0/1). 5 (70 mg, 129.25 μmol, 32% yield) was obtained as a yellow solid.

LCMS: RT=1.331 min, MS cal.: 541.2, [M+H]⁺=542.1

¹H NMR (400 MHz, DMSO-d₆) δ=7.88 (d, J=10.0 Hz, 1H), 7.69 (d, J=3.6 Hz, 2H), 7.64 (t, J=7.6 Hz, 1H), 7.60 (d, J=8.4 Hz, 1H), 7.20 (d, J=8.8 Hz, 1H), 6.87 (d, J=7.2 Hz, 1H), 6.71 (d, J=8.0 Hz, 1H), 5.46 (s, 2H), 4.62-4.57 (m, 2H), 4.53 (d, J=4.8 Hz, 2H), 3.85 (s, 2H), 3.81 (s, 3H), 2.91 (d, J=11.2 Hz, 2H), 2.58 (s, 1H), 2.23-2.14 (m, 2H), 1.82-1.67 (m, 4H)

### General Procedure for Preparation of Compound 104 (6):

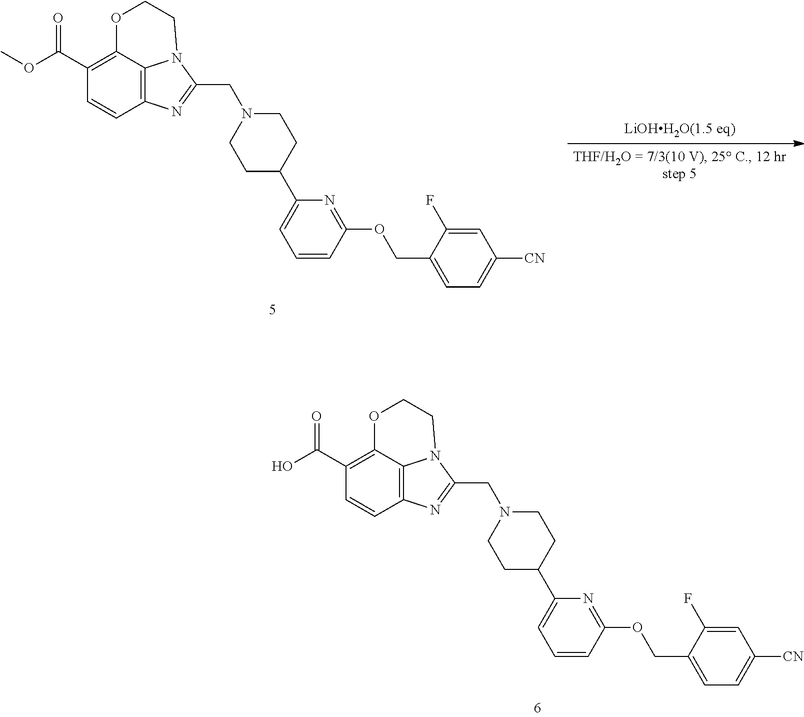

A mixture of 5 (70 mg, 129.25 μmol, 1 eq), LiOH·H₂O (8.14 mg, 193.88 μmol, 1.5 eq) in THF (0.7 mL) and H₂O (0.3 mL) was degassed and purged with N₂ for 3 times, and then the mixture was stirred at 25° C. for 12 hr under N₂ atmosphere. LCMS (RT=1.396) showed 5 was consumed completely. The reaction mixture was filtered. The filtrate was purified by prep-HPLC (column: Waters Xbridge Prep OBD C18 150*40 mm*10 um; mobile phase: [H₂O (10 mM NH₄HCO₃)-ACN]; gradient: 15%-65% B over 8.0 min). 2-((4-(6-((4-Cyano-2-fluorobenzyl)oxy)pyridin-2-yl)piperidin-1-yl)methyl)-3,4-dihydro-5-oxa-1,2a-diazaacenaphthylene-6-carboxylic acid (28.15 mg, 52.60 μmol, 41% yield, 98.57% purity) was obtained as a white solid.

LCMS: RT=2.449 min, MS cal.: 527.2; 52802, [M+H]⁺=528.2

HPLC: RT=9.078 min, purity: 98.57%

¹H NMR (400 MHz, METHANOL-d₄) δ=7.75 (d, J=8.8 Hz, 1H), 7.69-7.63 (m, 1H), 7.62-7.55 (m, 2H), 7.53 (dd, J=1.2, 8.0 Hz, 1H), 7.20 (d, J=8.4 Hz, 1H), 6.84 (d, J=7.2 Hz, 1H), 6.68 (d, J=8.0 Hz, 1H), 5.51 (s, 2H), 4.65-4.57 (m, 4H), 3.93 (s, 2H), 3.04 (d, J=11.6 Hz, 2H), 2.63 (t, J=7.2 Hz, 1H), 2.37-2.26 (m, 2H), 1.91-1.80 (m, 4H)

### Example A275—Preparation of Compound 113. 2-((4-(6-((4-Cyano-2-fluorobenzyl)oxy)pyridin-2-yl)piperidin-1-yl)methyl)-4-(fluoromethoxy)-1-methyl-1H-benzo[d]imidazole-6-carboxylic acid (Compound 113) (9)

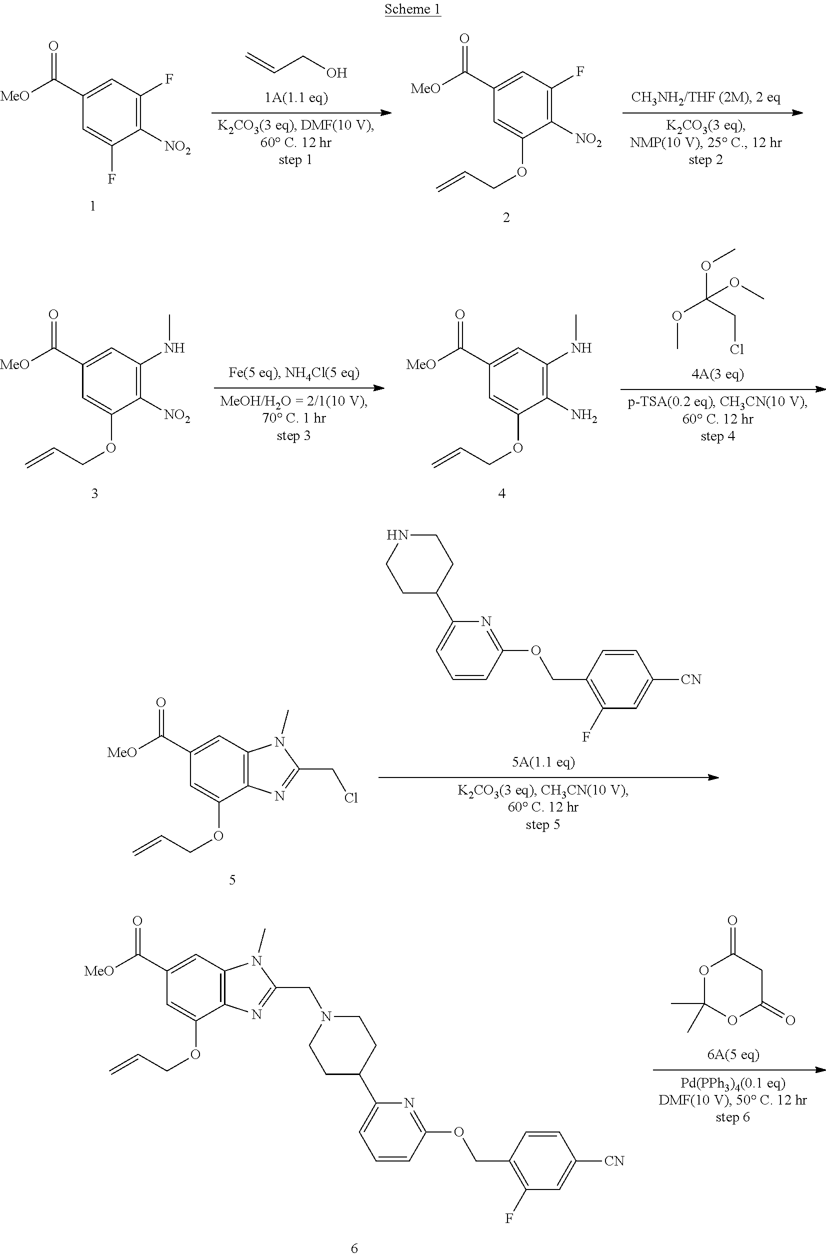

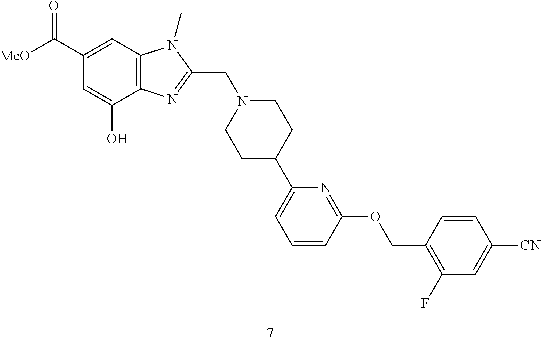 [SMILES: COC(=O)c1cc(O)c2nc(CN3CCC(c4cccc(OCc5ccc(C#N)cc5F)n4)CC3)n(C)c2c1]

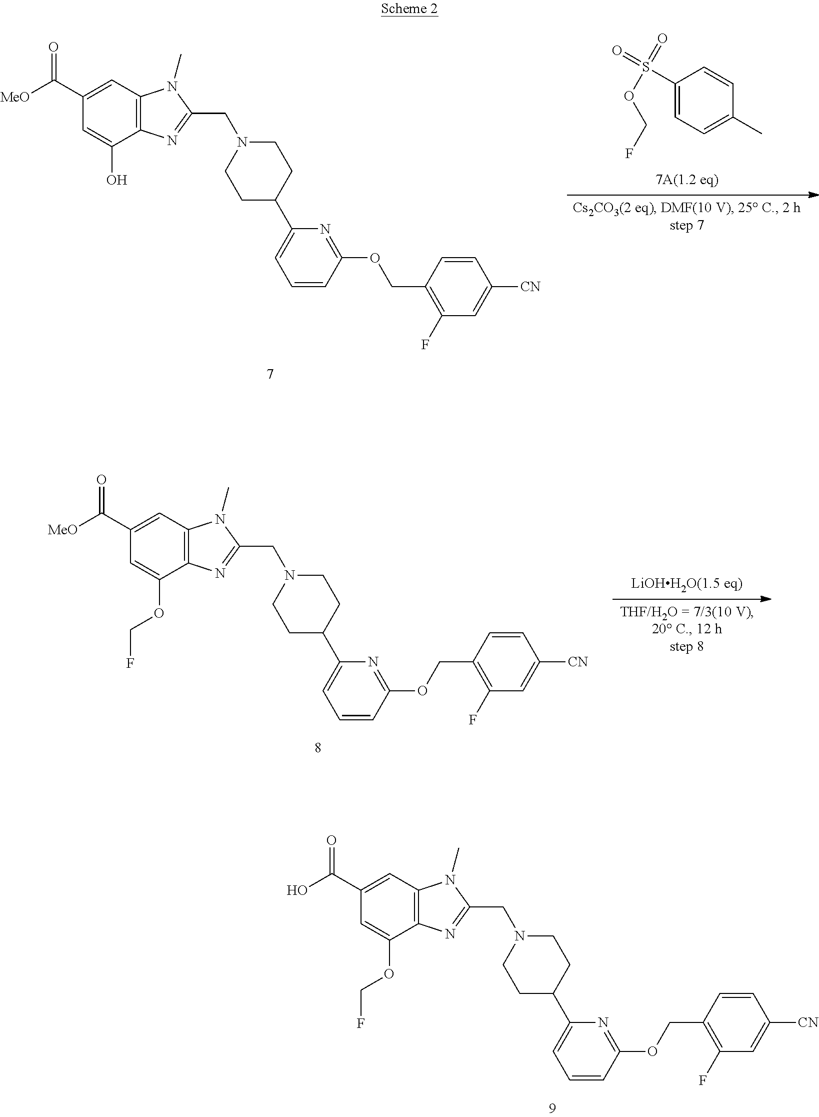

### General Procedure for Preparation of Scheme 1, Intermediate 2:

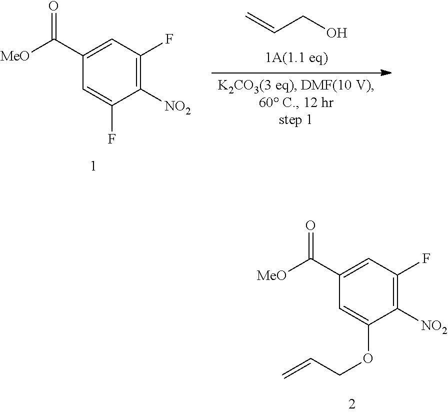

DMF (50 mL) was charged to the 100 mL three-necked round bottom, then 1 (5 g, 23.03 mmol, 1 eq) and K₂CO₃ (9.55 g, 69.08 mmol, 3 eq) was added to the mixture at 25° C. At 25° C., 1A (1.47 g, 25.33 mmol, 1.72 mL, 1.1 eq) was added dropwise to the reaction mixture at 25° C. After the addition, the mixture was stirred at 60° C. for 12 hr. TLC (Petroleum ether:Ethyl acetate=3:1, Rf=0.50) indicated 1 was consumed completely and one new spot formed. After 12 hr, the reaction mixture was quenched by H₂O (100 mL) at 20° C. The mixture was diluted with H₂O (50 mL) and extracted by EtOAc 150 mL (50 mL*3). Then the organic phase was combined and washed by brine (50 mL), dried with Na₂SO₄, filtered. The organic phase was concentrated under reduced pressure at 40° C. to give a residue. The residue was purified by column chromatography (SiO₂, Petroleum ether/Ethyl acetate=10/1 to 1/1). Compound 2 (6 g, 23.51 mmol, 51.05% yield) was obtained as yellow oil.

¹H NMR (400 MHz, DMSO-d₆) δ=7.67-7.62 (m, 2H), 6.01 (tdd, J=5.2, 10.4, 17.2 Hz, 1H), 5.41-5.29 (m, 2H), 4.91-4.88 (m, 2H), 3.91 (s, 3H)

### General Procedure for Preparation of Scheme 1, Intermediate 3:

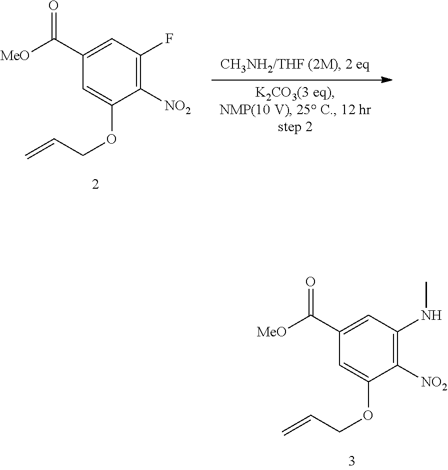 [SMILES: C=CCOc1cc(C(=O)OC)cc(F)c1[N+](=O)[O-] ; "CH3NH2/THF (2M), 2 eq" ; "K2CO3(3 eq),NMP(10 V), 25° C., 12 hrstep 2" ; C=CCOc1cc(C(=O)OC)cc(NC)c1[N+](=O)[O-]]

NMP (30 mL) was charged to the 100 mL three-necked round bottom flask, then 2 (3 g, 11.76 mmol, 1 eq), K₂CO₃ (4.87 g, 35.27 mmol, 3 eq) was added to the mixture at 25° C. At 25° C., CH₃NH₂ (2 M in THF, 11.76 mL, 2 eq) was added dropwise to the reaction mixture at 25° C. After the addition, the mixture was stirred at 25° C. for 12 hr. TLC showed 1 was consumed completely. The reaction mixture was added with FA (1 M) to adjust pH=5 at 25° C. The mixture was extracted by EtOAc (30 mL). Then organic phase was combined and washed by H₂O (30 mL), brine (20 mL), dried with Na₂SO₄, filtered. The organic phase was concentrated under reduced pressure at 45° C. to give a residue. 3 (3 g, 11.27 mmol, 95.85% yield) was obtained as a yellow solid.

¹H NMR (400 MHz, DMSO-d₆) δ=6.91 (dd, J=1.2, 14.0 Hz, 2H), 6.35 (br d, J=4.4 Hz, 1H), 5.97 (br dd, J=10.8, 17.2 Hz, 1H), 5.35 (dd, J=1.6, 17.3 Hz, 1H), 5.26 (dd, J=1.6, 10.4 Hz, 1H), 4.70 (d, J=4.8 Hz, 2H), 3.86 (s, 3H), 2.76 (d, J=4.8 Hz, 3H)

### General Procedure for Preparation of Scheme 1, Intermediate 4:

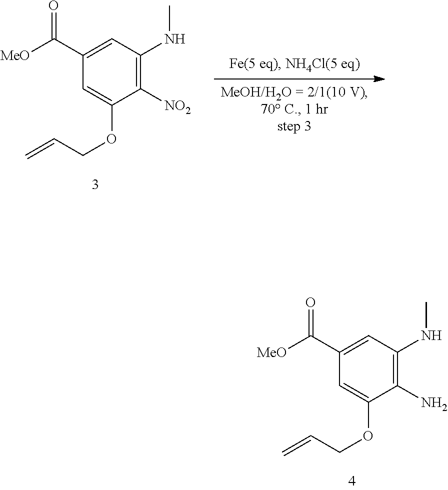

MeOH (30 mL) and H₂O (15 mL) were charged to the three-necked round bottom flask, then 3 (3 g, 11.27 mmol, 1 eq) and NH₄Cl (3.01 g, 56.34 mmol, 5 eq) was added to the mixture at 25° C. At 25° C., Fe (3.15 g, 56.34 mmol, 5 eq) was added in portions to the reaction mixture at 25° C. After the addition, the mixture was stirred at 70° C. for 1 h. TLC (PE:EA=3:1, Rf=0.27) indicated 3 was consumed completely. The reaction mixture was filtered and concentrated under reduced pressure. The mixture was extracted by EtOAc (30 mL*3). Then organic phase was combined and washed by H₂O (30 mL), brine (30 mL), dried with Na₂SO₄, filtered. The organic phase was concentrated under reduced pressure at 40° C. to give a residue. 4 (2.5 g, 10.58 mmol, 93.91% yield) was obtained as yellow oil.

LCMS: RT=0.330 min, MS cal.: 236.1, [M+H]⁺=237

### General Procedure for Preparation of Scheme 1, Intermediate 5:

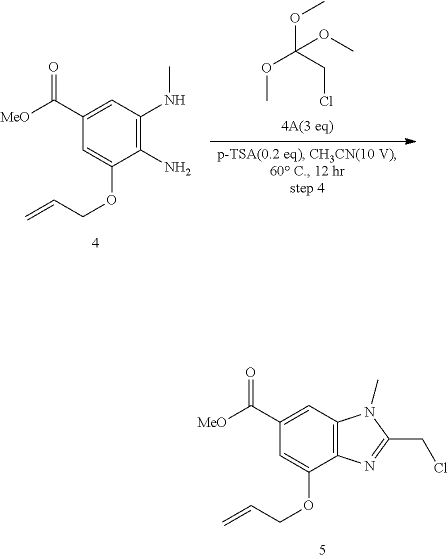

A mixture of 4 (2.5 g, 10.58 mmol, 1 eq), 4A (4.91 g, 31.74 mmol, 4.28 mL, 3 eq), TosOH (182.21 mg, 1.06 mmol, 0.1 eq) in CH₃CN (25 mL) was degassed and purged with N₂ for 3 times, and then the mixture was stirred at 60° C. for 2 hr under N₂ atmosphere. LCMS showed 4 was consumed completely and one main peak with desired mass was detected. The reaction mixture was filtered and concentrated under reduced pressure to give a residue. 5 (3 g, 10.18 mmol, 96.20% yield) was obtained as yellow oil.

LCMS: RT=0.448 min, MS cal.: 294.1, [M+H]⁺=395.0

### General Procedure for Preparation of Scheme 1, Intermediate 6:

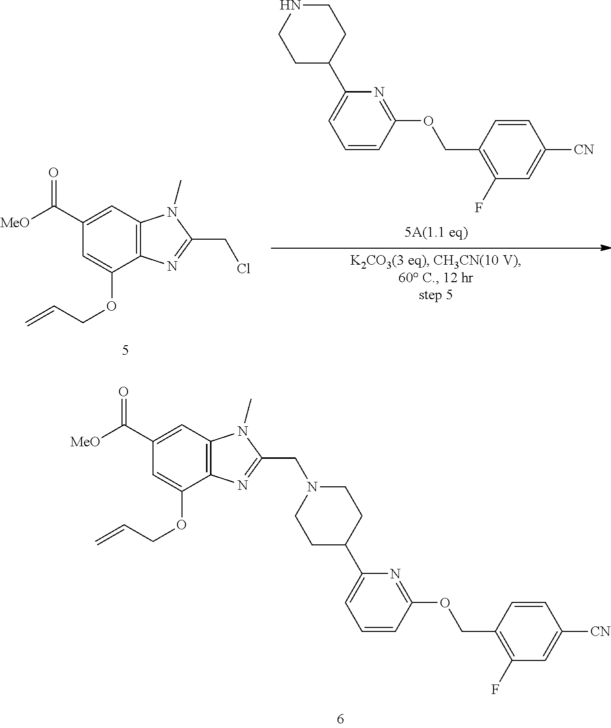 [SMILES: C=CCOc1cc(C(=O)OC)cc2c1nc(CCl)n2C ; N#Cc1ccc(COc2cccc(C3CCNCC3)n2)c(F)c1 ; "5A(1.1 eq)" ; "K2CO3(3 eq), CH3CN(10 V),60° C., 12 hrstep 5" ; C=CCOc1cc(C(=O)OC)cc2c1nc(CN1CCC(c3cccc(OCc4ccc(C#N)cc4F)n3)CC1)n2C]

A mixture of 5 (3 g, 10.18 mmol, 1 eq), 5A (3.49 g, 11.20 mmol, 1.1 eq), K₂CO₃ (4.22 g, 30.54 mmol, 3 eq) in CH₃CN (30 mL) was degassed and purged with N₂ for 3 times, and then the mixture was stirred at 60° C. for 12 hr under N₂ atmosphere. LCMS showed 5 was consumed completely and one main peak with desired mass was detected. The reaction mixture was diluted with H₂O (30 mL) and extracted with EtOAc (30 mL*3). The combined organic layers were washed with brine (30 mL*3), dried over Na₂SO₄, filtered, and concentrated under reduced pressure to give a residue. The residue was purified by column chromatography (SiO₂, Petroleum ether/Ethyl acetate=10/1 to 0/1). 6 (4 g, 7.02 mmol, 68.99% yield) was obtained as a yellow solid.

LCMS: RT=0.435 min, MS cal.: 569.2, [M+H]⁺=570.6

¹H NMR (400 MHz, DMSO-d6) δ=7.90-7.80 (m, 2H), 7.69 (d, J=3.2 Hz, 2H), 7.63 (t, J=7.6 Hz, 1H), 7.28 (s, 1H), 6.87 (d, J=7.2 Hz, 1H), 6.71 (d, J=8.0 Hz, 1H), 6.18-6.06 (m, 1H), 5.45 (s, 2H), 5.49-5.42 (m, 1H), 5.29 (dd, J=1.2, 10.6 Hz, 1H), 4.83 (br d, J=5.2 Hz, 2H), 3.92 (s, 3H), 3.89-3.85 (m, 3H), 3.81 (s, 2H), 2.90 (br d, J=11.2 Hz, 2H), 2.58-2.52 (m, 1H), 2.18 (br t, J=10.8 Hz, 2H), 1.78-1.58 (m, 4H)

### General Procedure for Preparation of Scheme 1, Intermediate 7:

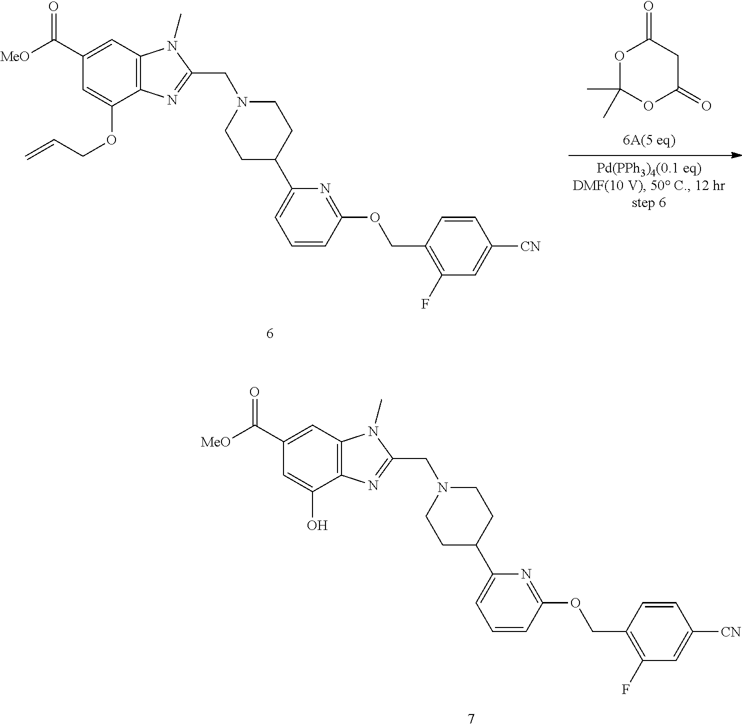

A mixture of 6 (4 g, 7.02 mmol, 1 eq), 6A (5.06 g, 35.11 mmol, 5 eq), Pd(PPh₃)₄ (811.45 mg, 702.22 μmol, 0.1 eq) in DMF (40 mL) was degassed and purged with N₂ for 3 times, and then the mixture was stirred at 50° C. for 12 hr under N₂ atmosphere. LCMS showed 6 was consumed completely and one main peak with desired mass was detected. The reaction mixture was diluted with H₂O (50 mL) and extracted with EtOAc (50 mL*3). The combined organic layers were washed with brine (50 mL*3), dried over Na₂SO₄, filtered, and concentrated under reduced pressure to give a residue. The residue was purified by prep-HPLC (column: Waters Xbridge BEH C18 250*50 mm*10 um; mobile phase: [H₂O (10 mM NH₄HCO₃)-ACN]; gradient: 30%-70% B over 10.0 min). 7 (1.5 g, 2.83 mmol, 40.34% yield) was obtained as a yellow solid.

LCMS: Rt=2.133 min, MS cal.: 529.2, [M+H]⁺=530.2

¹H NMR (400 MHz, METHANOL-d4) δ=7.72 (d, J=1.2 Hz, 1H), 7.67 (s, 1H), 7.61-7.51 (m, 3H), 6.83 (d, J=7.6 Hz, 1H), 6.67 (d, J=8.0 Hz, 1H), 5.50 (s, 2H), 3.96 (s, 3H), 3.92 (s, 3H), 3.86 (s, 2H), 3.00 (br d, J=11.6 Hz, 2H), 2.67-2.56 (m, 1H), 2.28 (br s, 2H), 1.86-1.76 (m, 4H)

### General Procedure for Preparation of Scheme 2, Intermediate 8:

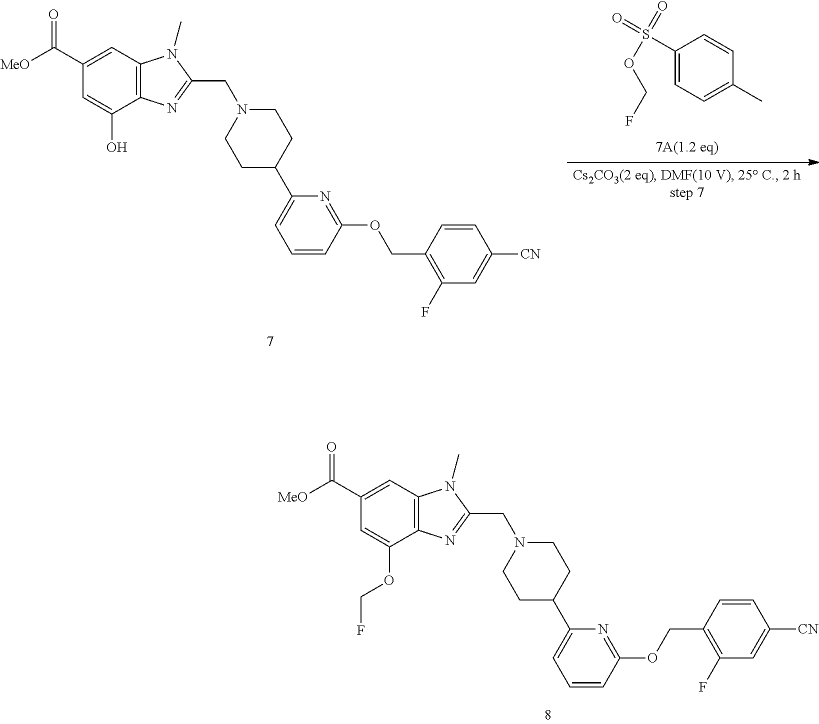 [SMILES: COC(=O)c1cc(O)c2nc(CN3CCC(c4cccc(OCc5ccc(C#N)cc5F)n4)CC3)n(C)c2c1 ; Cc1ccc(S(=O)(=O)OCF)cc1 ; "7A(1.2 eq)" ; "Cs2CO3(2 eq), DMF(10 V), 25° C., 2 hstep 7" ; COC(=O)c1cc(OCF)c2nc(CN3CCC(c4cccc(OCc5ccc(C#N)cc5F)n4)CC3)n(C)c2c1]

A mixture of 7 (90 mg, 169.95 μmol, 1 eq), 7A (41.65 mg, 203.94 μmol, 1.2 eq), Cs₂CO₃ (110.75 mg, 339.90 μmol, 2 eq) in DMF (1 mL) was degassed and purged with N₂ for 3 times, and then the mixture was stirred at 25° C. for 12 hr under N₂ atmosphere. LCMS showed 7 was consumed completely and one main peak with desired mass was detected. The reaction mixture was diluted with H₂O (5 mL) and extracted with EtOAc (5 mL*3). The combined organic layers were washed with brine (5 mL*3), dried over Na₂SO₄, filtered, and concentrated under reduced pressure to give a residue. The residue was purified by column chromatography (SiO₂, Petroleum ether/Ethyl acetate=10/1 to 0/1). 8 (80 mg, 142.46 μmol, 83.82% yield) was obtained as yellow oil.

LCMS: RT=0.442 min, MS cal.: 561.2, [M+H]⁺=562.2

¹H NMR (400 MHz, METHANOL-d4) δ=8.05 (d, J=1.2 Hz, 1H), 7.68 (s, 2H), 7.63-7.52 (m, 3H), 6.84 (d, J=7.2 Hz, 1H), 6.69 (d, J=8.0 Hz, 1H), 6.08-5.92 (m, 2H), 5.52 (s, 2H), 4.04 (s, 3H), 3.97 (s, 3H), 3.91 (s, 2H), 3.01 (s, 3H), 2.70-2.58 (m, 1H), 2.36-2.26 (m, 2H), 1.87-1.78 (m, 4H)

### General Procedure for Preparation of Compound 113 (9):

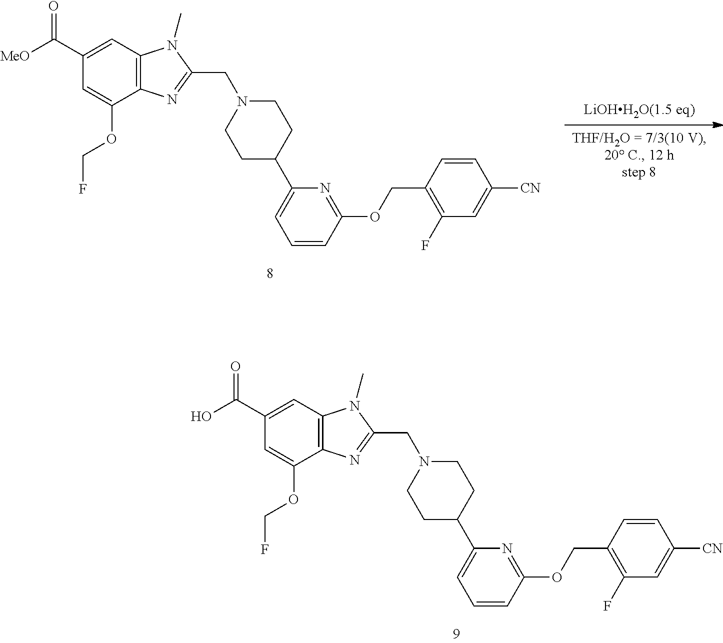

A mixture of 8 (80 mg, 142.46 μmol, 1 eq), LiOH·H₂O (8.97 mg, 213.68 μmol, 1.5 eq) in THF (0.7 mL) and H₂O (0.3 mL) was degassed and purged with N₂ for 3 times, and then the mixture was stirred at 25° C. for 12 hr under N₂ atmosphere. LCMS showed 8 was consumed completely and one main peak with desired mass was detected. The reaction mixture was filtered and concentrated under reduced pressure to give a residue in DMSO. The residue was purified by prep-HPLC (column: Waters Xbridge Prep OBD C18 150*40 mm*10 um; mobile phase: [H₂O (10 mM NH₄HCO₃)-ACN]; gradient: 15%-65% B over 8.0 min). 2-((4-(6-((4-Cyano-2-fluorobenzyl)oxy)pyridin-2-yl)piperidin-1-yl)methyl)-4-(fluoromethoxy)-1-methyl-1H-benzo[d]imidazole-6-carboxylic acid (27.2 mg, 49.68 μmol, 34.87% yield) was obtained as a white solid.

LCMS: RT=2.569 min, MS cal.: 547.2, [M+H]⁺=548.2

HPLC: RT=9.921 min, purity: 97.79%

¹H NMR (400 MHz, DMSO-d₆) δ=7.96 (s, 1H), 7.89 (d, J=10.0 Hz, 1H), 7.70 (d, J=3.6 Hz, 2H), 7.65 (t, J=7.6 Hz, 1H), 7.50 (s, 1H), 6.88 (d, J=7.2 Hz, 1H), 6.72 (d, J=8.0 Hz, 1H), 6.22-6.03 (m, 2H), 5.46 (s, 2H), 3.95 (s, 3H), 3.83 (s, 2H), 2.92 (br d, J=11.2 Hz, 2H), 2.63-2.56 (m, 1H), 2.20 (br t, J=10.8 Hz, 2H), 1.81-1.59 (m, 4H)

### Example A276—Preparation of Compound 386 and Compound 71. 2-((4-((S)-2-(5-Chloropyridin-2-yl)-2-methylbenzo[d][1,3]dioxol-4-yl)piperidin-1-yl)methyl)-4-(1,1-difluoroethyl)-1-(((S)-oxetan-2-yl)methyl)-1H-benzo[d]imidazole-6-carboxylic acid (Compound 386 (10A) & 2-((4-((R)-2-(5-Chloropyridin-2-yl)-2-methylbenzo[d][1,3]dioxol-4-yl)piperidin-1-yl)methyl)-4-(1,1-difluoroethyl)-1-(((S)-oxetan-2-yl)methyl)-1H-benzo[d]imidazole-6-carboxylic acid (Compound 71) (10B)

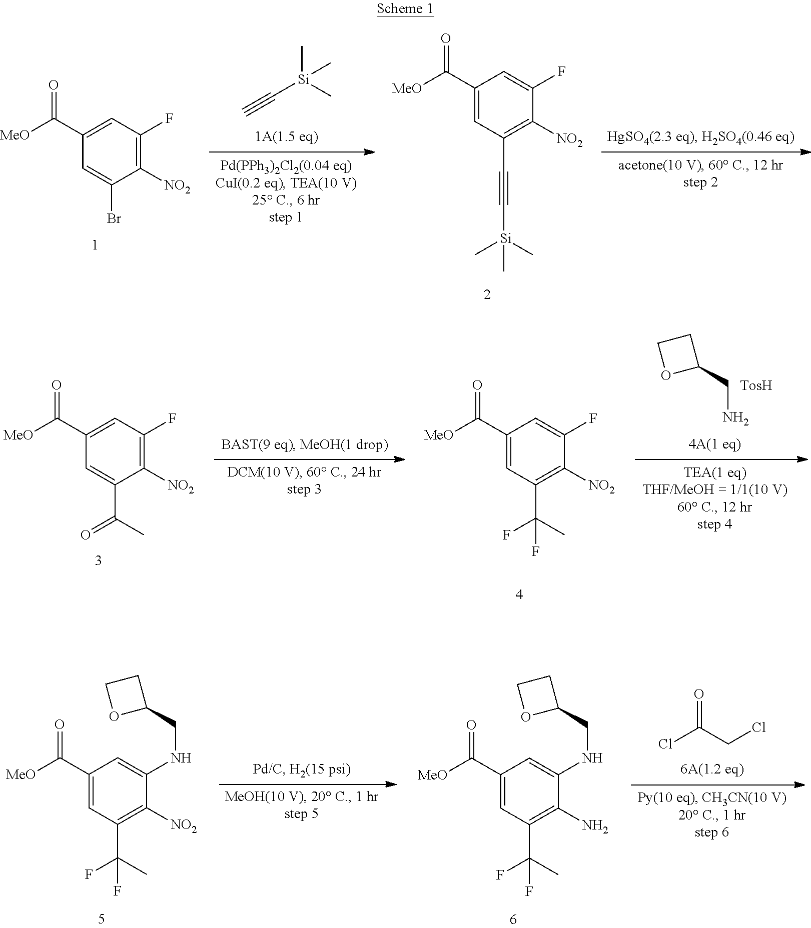

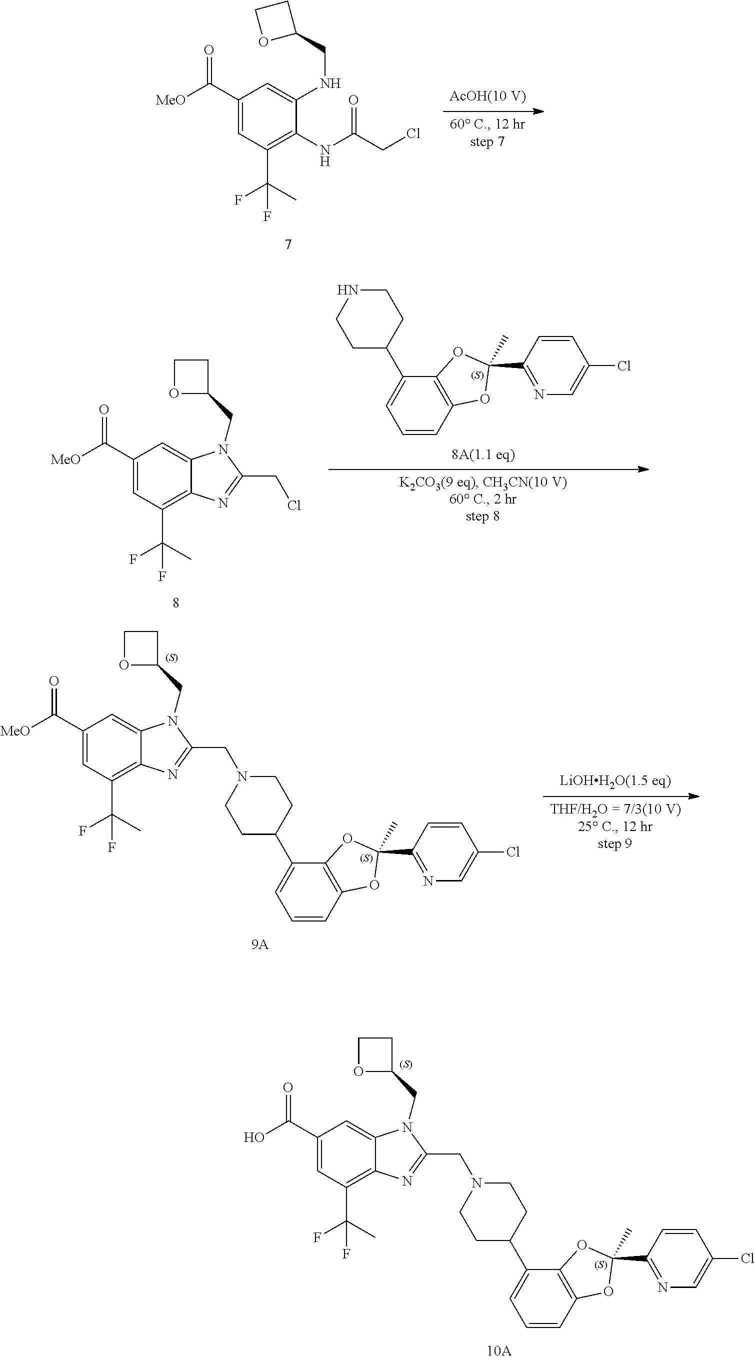

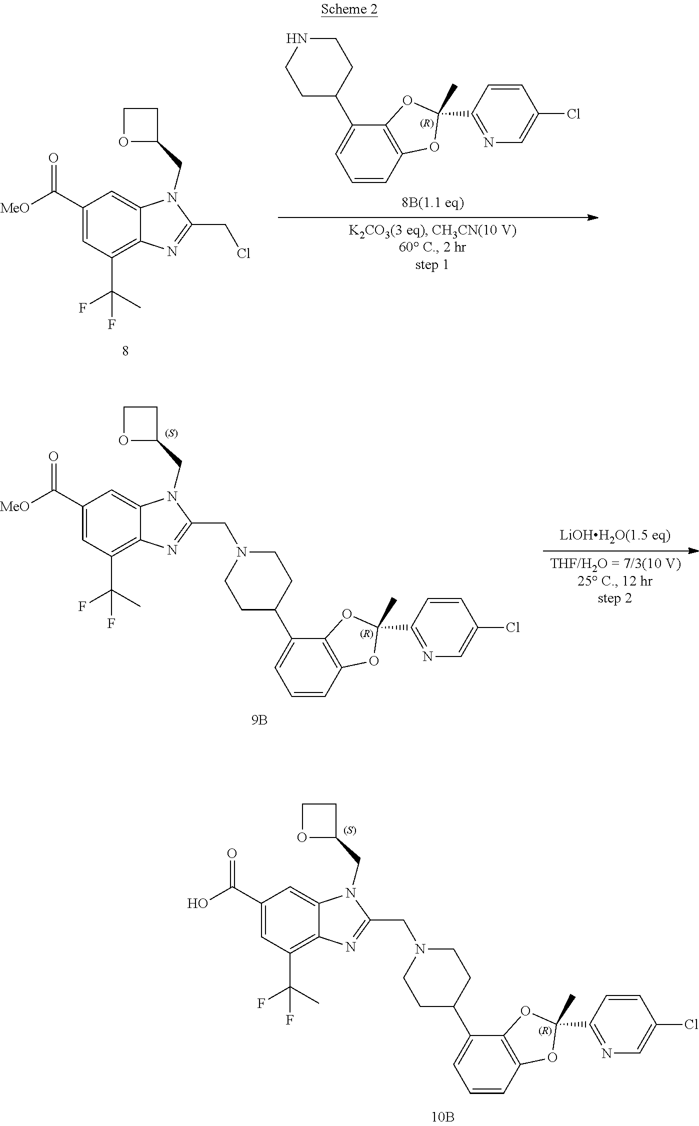

### General Procedure for Preparation of Scheme 1, Intermediate 2:

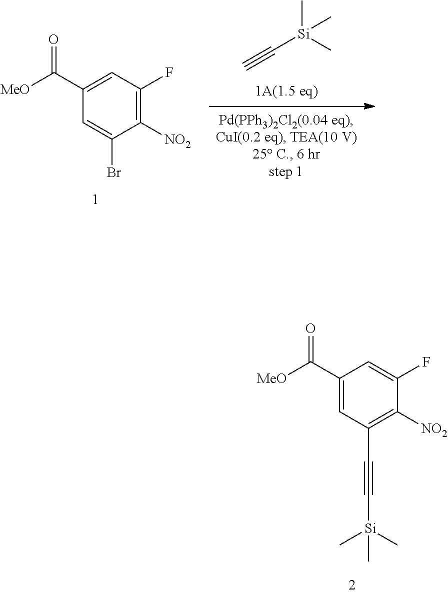

A mixture of 1 (500 mg, 1.80 mmol, 1 eq), Pd(PPh₃)₂Cl₂ (50.49 mg, 71.93 μmol, 0.04 eq) in TEA (10 mL) was degassed and purged with N₂ for 3 times. After addition, the mixture was stirred at 25° C. for 10 min, and then 1A (264.95 mg, 2.70 mmol, 373.69 μL, 1.5 eq) and CuI (68.50 mg, 359.67 μmol, 0.2 eq) in TEA (10 mL) was added at 25° C. The resulting mixture was stirred at 25° C. for 6 hr. TLC (Petroleum ether/Ethyl acetate=5/1, R_{f}=0.4) indicated 1 was consumed completely and one new spot formed. The reaction mixture was diluted with water (100 mL) and extracted with EtOAc (70 mL*3). The organic phase was washed with brine (25 mL), dried over Na₂SO₄, filtered and concentrated under reduced pressure to give a residue. The residue was purified by column chromatography (SiO₂, Petroleum ether/Ethyl acetate=20/1 to 0/1). 2 (2.7 g, 9.14 mmol, 72% yield) was obtained as a yellow solid.

¹H NMR (400 MHz, CHLOROFORM-d) δ=8.04 (s, 1H), 7.85 (dd, J=1.2, 9.6 Hz, 1H), 3.98 (s, 3H), 0.26 (s, 9H)

### General Procedure for Preparation of Scheme 1, Intermediate 3:

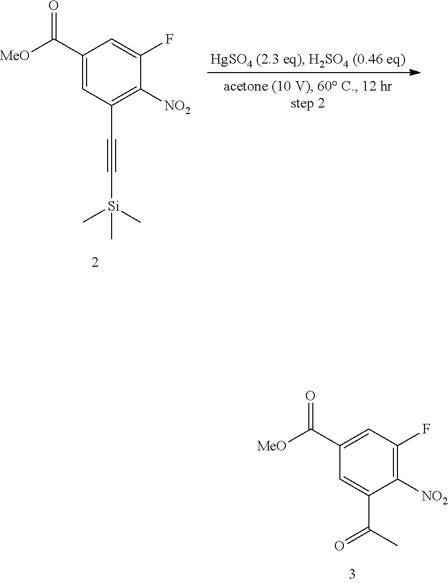 [SMILES: COC(=O)c1cc(F)c([N+](=O)[O-])c(C#C[Si](C)(C)C)c1 ; COC(=O)c1cc(F)c([N+](=O)[O-])c(C(C)=O)c1]

A mixture of 2 (4 g, 13.54 mmol, 1 eq), HgSO₄ (9.24 g, 31.15 mmol, 2.3 eq), H₂SO₄ (611.04 mg, 6.23 mmol, 332.09 μL, 0.46 eq) in acetone (50 mL) was degassed and purged with N₂ for 3 times, and then the mixture was stirred at 60° C. for 12 hr under N₂ atmosphere. TLC (Petroleum ether/Ethyl acetate=1/1, R_{f}=0.3) indicated 2 was consumed completely and one new spot formed. The reaction mixture was diluted with water (100 mL) and extracted with EtOAc (100 mL*3). The organic phase was washed with brine (50 mL*2), dried over Na₂SO₄, filtered and concentrated under reduced pressure to give a residue. The residue was purified by column chromatography (SiO₂, Petroleum ether/Ethyl acetate=9/1 to 0/1). 3 (2.4 g, 9.95 mmol, 73.48% yield) was obtained as a brown solid.

LCMS: RT=1.622 min. MS cal.: 241.0; 242.0, [M−H]⁻=240.1; 240.9

¹H NMR (400 MHz, CHLOROFORM-d) δ=8.25 (t, J=1.2 Hz, 1H), 8.08 (dd, J=1.6, 8.8 Hz, 1H), 4.02 (s, 3H), 2.67 (s, 3H)

### General Procedure for Preparation of Scheme 1, Intermediate 4:

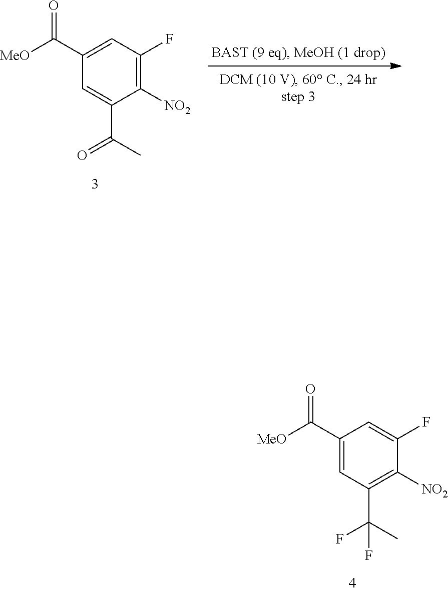 [SMILES: COC(=O)c1cc(F)c([N+](=O)[O-])c(C(C)=O)c1 ; COC(=O)c1cc(F)c([N+](=O)[O-])c(C(C)(F)F)c1]

A mixture of 3 (500 mg, 2.07 mmol, 1 eq), BAST (4.13 g, 18.66 mmol, 4.09 mL, 9 eq) and MeOH (33.21 mg, 1.04 mmol, 41.95 μL, 0.5 eq) in DCM (25 mL) was degassed and purged with N₂ for 3 times, and then the mixture was stirred at 60° C. for 24 hr under N₂ atmosphere. HPLC (RT=3.092) showed 3 consumed completely. Several new peaks were shown on HPLC and ˜77% of desired compound was detected. Meanwhile TLC (Petroleum ether/Ethyl acetate=2/1, R_{f}=0.4) indicated 3 was consumed completely and one new spot formed. At 0° C., the reaction solution was slowly added to water (20 mL). The mixture was extracted with DCM (30 mL*3). The organic phase was washed with brine (15 mL*2), dried over Na₂SO₄, filtered and concentrated under reduced pressure to give a residue. The residue was purified by column chromatography (SiO₂, Petroleum ether/Ethyl acetate=20/1 to 0/1). 4 (800 mg, 3.04 mmol, 73% yield) was obtained as a yellow solid.

HPLC: RT=3.092 min, purity=87%

¹H NMR (400 MHz, CHLOROFORM-d) δ=8.07 (s, 1H), 8.01 (d, J=8.8 Hz, 1H), 4.01 (s, 3H), 2.05 (t, J=18.4 Hz, 3H)

### General Procedure for Preparation of Scheme 1, Intermediate 5:

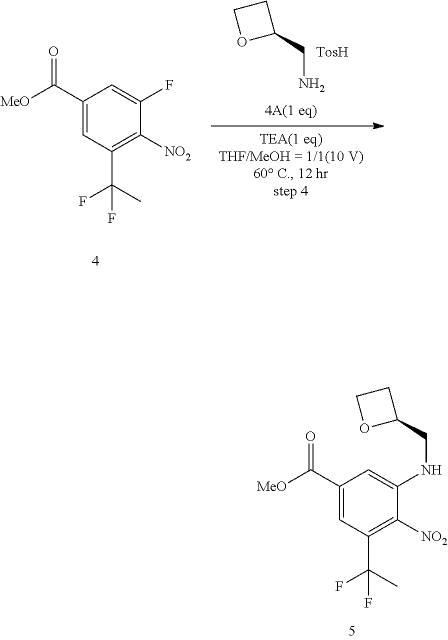

A mixture of 4 (1 g, 3.80 mmol, 1 eq), 4A (985.38 mg, 3.80 mmol, 1 eq, p-TSA), TEA (384.50 mg, 3.80 mmol, 528.89 μL, 1 eq) in MeOH (5 mL) and THF (5 mL) was degassed and purged with N₂ for 3 times, and then the mixture was stirred at 60° C. for 12 hr under N₂ atmosphere. TLC (Petroleum ether/Ethyl acetate=1/1, R_{f}=0.2) indicated 4 was consumed completely and one new spot formed. The reaction mixture was diluted with water (10 mL) and extracted with DCM (10 mL*3). The organic phase was washed with brine (5 mL*2), dried over Na₂SO₄, filtered and concentrated under reduced pressure to give a residue. The residue was purified by column chromatography (SiO₂, Petroleum ether/Ethyl acetate=9/1 to Petroleum ether/MeOH=0/1). 5 (0.6 g, 1.82 mmol, 47% yield) was obtained as an orange oil.

LCMS: RT=0.508 min, MS cal.: 330.1; 331.1, [M+H]⁺=331.2; 332.3

¹H NMR (400 MHz, CHLOROFORM-d) δ=7.61 (s, 1H), 7.52 (s, 1H), 5.40 (t, J=4.8 Hz, 1H), 5.15-4.98 (m, 1H), 4.79-4.68 (m, 1H), 4.63-4.52 (m, 1H), 3.96 (s, 3H), 3.58-3.45 (m, 2H), 2.81-2.66 (m, 1H), 2.63-2.50 (m, 1H), 2.19-2.00 (t, J=18.4 Hz, 3H)

### General Procedure for Preparation of Intermediate 6:

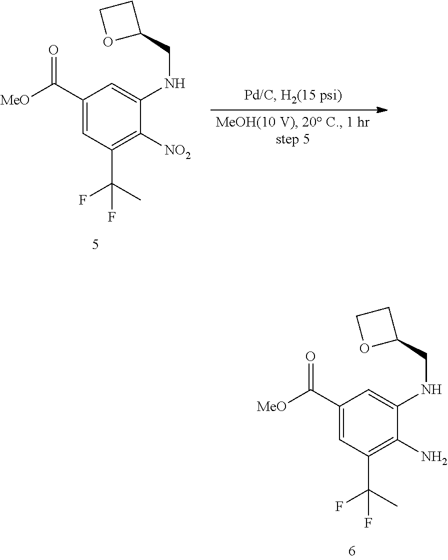 [SMILES: COC(=O)c1cc(NC[C@@H]2CCO2)c([N+](=O)[O-])c(C(C)(F)F)c1 ; "MeOH(10 V), 20° C., 1 hrstep 5" ; "Pd/C, H2(15 psi)" ; COC(=O)c1cc(NC[C@@H]2CCO2)c(N)c(C(C)(F)F)c1]

To a solution of Pd/C (275.49 mg) in MeOH (6 mL) was added 5 (570 mg, 1.73 mmol, 1 eq) under N₂ atmosphere. The suspension was degassed and purged with H₂ for 3 times. The mixture was stirred under H₂ (15 psi) at 20° C. for 1 hr. LCMS (RT=1.565) showed 5 was consumed completely and one main peak with desired mass was detected. The reaction mixture was filtered, the filter cake was washed with MeOH (15 mL*3) and the combined filtrate was concentrated under reduced pressure to give a product. 6 (518 mg, crude) was obtained as a brown solid.

¹H NMR (400 MHz, CHLOROFORM-d) δ=7.68 (s, 1H), 7.47 (s, 1H), 5.12-5.11 (m, 1H), 4.77-4.75 (m, 1H), 4.68-4.54 (m, 1H), 4.57 (s, 2H), 3.88 (s, 3H), 3.55-3.39 (m, 2H), 3.38-3.30 (m, 1H), 2.77-2.75 (m, 1H), 2.61-2.59 (m, 1H), 2.02 (t, J=18.8 Hz, 3H)

### General Procedure for Preparation of Scheme 1, Intermediate 7:

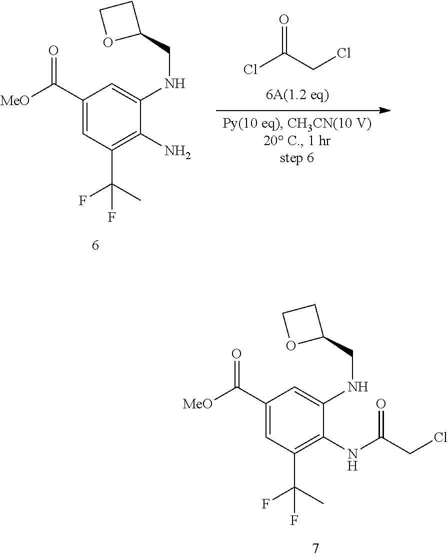

A mixture of 6 (500 mg, 1.66 mmol, 1 eq), Py (1.32 g, 16.65 mmol, 1.34 mL, 10 eq) and 6A (225.65 mg, 2.00 mmol, 159.14 μL, 1.2 eq) in CH₃CN (5 mL) was degassed and purged with N₂ for 3 times, and then the mixture was stirred at 20° C. for 1 hr under N₂ atmosphere. TLC (Petroleum ether/Ethyl acetate=1/1, R_{f}=0.3) indicated 6 was consumed completely and one new spot formed. The reaction mixture was diluted with water (10 mL) and extracted with DCM (10 mL*3). The combined organic phase was dried over Na₂SO₄, filtered and concentrated under reduced pressure to give a residue. The residue was purified by column chromatography (SiO₂, Petroleum ether/Ethyl acetate=5/1 to 0/1). 7 (300 mg, 796.22 μmol, 47% yield) was obtained as a yellow solid.

LCMS: RT=1.537 min, MS cal.: 376.1; 378.1, [M+H]⁺=377.1; 379.1

### General Procedure for Preparation of Scheme 1, Intermediate 8:

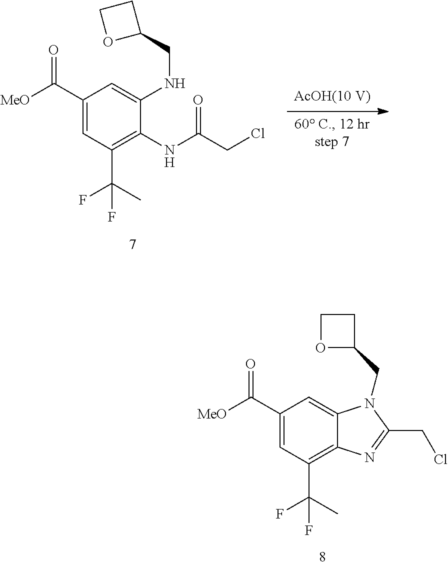 [SMILES: COC(=O)c1cc(NC[C@@H]2CCO2)c(NC(=O)CCl)c(C(C)(F)F)c1 ; "60° C., 12 hrstep 7" ; "AcOH(10 V)" ; COC(=O)c1cc(C(C)(F)F)c2nc(CCl)n(C[C@@H]3CCO3)c2c1]

A mixture of 7 (300 mg, 796.22 μmol, 1 eq) in AcOH (1.5 mL) was degassed and purged with

N₂ for 3 times, and then the mixture was stirred at 60° C. for 12 hr under N₂ atmosphere. LCMS (RT=0.467) showed 7 was consumed completely and one main peak with desired mass was detected. The reaction mixture was concentrated under reduced pressure to remove AcOH. The residue was diluted with water (5 mL) and extracted with DCM (5 mL*3). The organic phase was dried over Na₂SO₄, filtered and concentrated under reduced pressure to give a residue. The residue was purified by column chromatography (SiO₂, Petroleum ether/Ethyl acetate=70/30 to Ethyl acetate/Methanol=0/1). 8 (230 mg, 641.08 μmol, 80% yield) was obtained as a white solid.

LCMS: RT=1.737 min, MS cal.: 358.1; 360.1, [M+H]⁺=359.1

¹H NMR (400 MHz, CHLOROFORM-d) δ=8.21 (s, 2H), 5.31-5.21 (m, 1H), 5.08 (s, 2H), 4.72-4.61 (m, 2H), 4.60-4.52 (m, 1H), 4.40-4.38 (m, 1H), 3.98 (s, 3H), 2.79 (dtd, J=6.0, 8.0, 11.6 Hz, 1H), 2.50-2.39 (m, 1H), 2.26 (t, J=18.8 Hz 3H)

### General Procedure for Preparation of Scheme 1, Intermediate 9A:

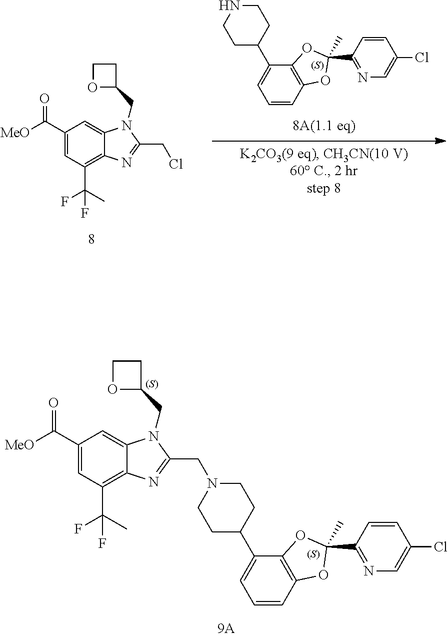

A mixture of 8 (120 mg, 334.48 μmol, 1 eq), 8A (121.71 mg, 367.93 μmol, 1.1 eq) and K₂CO₃ (416.04 mg, 3.01 mmol, 9 eq) in CH₃CN (1 mL) was degassed and purged with N₂ for 3 times, and then the mixture was stirred at 60° C. for 2 hr under N₂ atmosphere. LCMS (RT=0.489) showed 8 was consumed completely and one main peak with desired mass was detected. TLC (Petroleum ether/Ethyl acetate=0/1, R_{f}=0.2) indicated 8 was consumed completely and one new spot formed. The reaction mixture was diluted with water (5 mL) and extracted with DCM (5 mL*3). The organic phase was dried over Na₂SO₄, filtered and concentrated under reduced pressure to give a residue. The residue was purified by column chromatography (SiO₂, Petroleum ether/Ethyl acetate=7/3 to Ethyl acetate/Methanol=0/1). 9A (130 mg, 199.05 μmol, 59% yield) was obtained as a white solid.

LCMS: RT=2.393 min, MS cal.: 652.2; 653.2, [M+H]⁺=653.2

### General Procedure for Preparation of Compound 386 (10A):

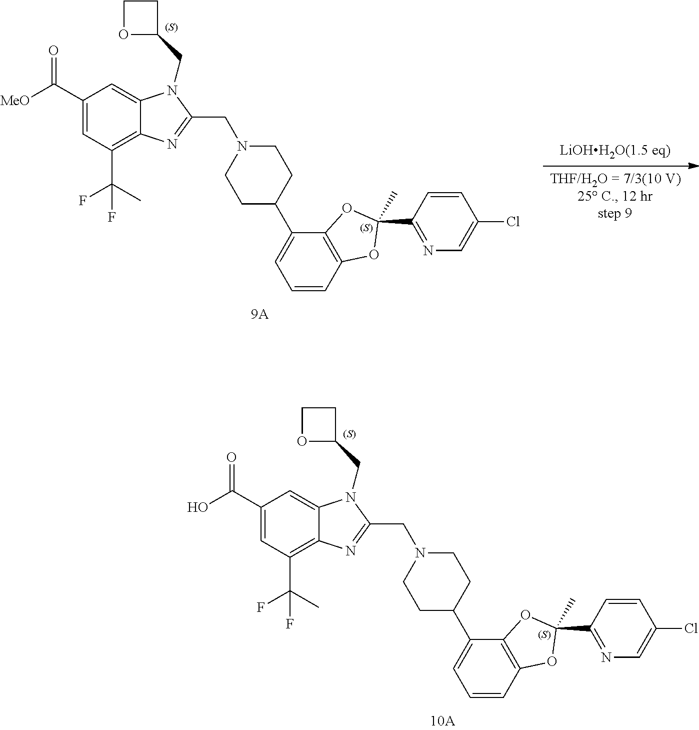

A mixture of 9A (0.1 g, 153.11 μmol, 1 eq), LiOH·H₂O (9.64 mg, 229.67 μmol, 1.5 eq) in THF (0.7 mL) and H₂O (0.3 mL) was degassed and purged with N₂ for 3 times, and then the mixture was stirred at 25° C. for 12 hr under N₂ atmosphere. LC-MS (RT=1.579) showed 9A was consumed completely and one main peak with desired mass was detected. The reaction mixture was filtered. The filtrate was purified by prep-HPLC (column: Waters Xbridge Prep OBD C18 150*40 mm*10 um; mobile phase: [H₂O (10 mM NH₄HCO₃)-ACN]; gradient: 30%-60% B over 8.0 min). 2-((4-((S)-2-(5-Chloropyridin-2-yl)-2-methylbenzo[d][1,3]dioxol-4-yl)piperidin-1-yl)methyl)-4-(1,1-difluoroethyl)-1-(((S)-oxetan-2-yl)methyl)-1H-benzo[d]imidazole-6-carboxylic acid (28.55 mg, 44.67 μmol, 29% yield) was obtained as a white solid.

LCMS: RT=2.742 min, MS cal.: 638.2; 639.2, [M+H]⁺=639.2

HPLC: RT=11.832 min

SFC: RT=4.923 min

¹H NMR (400 MHz, METHANOL-d₄) δ=8.61 (d, J=2.0 Hz, 1H), 8.41 (s, 1H), 8.12 (s, 1H), 7.87 (dd, J=2.4, 8.4 Hz, 1H), 7.65 (d, J=8.4 Hz, 1H), 6.83-6.76 (m, 1H), 6.75-6.70 (m, 2H), 5.27-5.25 (m, 1H), 4.93-4.87 (m, 1H), 4.73 (dd, J=2.4, 15.6 Hz, 1H), 4.66-4.58 (m, 1H), 4.50-4.48 (m, 1H), 4.21-4.15 (d, J=14.0 Hz, 1H), 4.10-4.04 (d, J=14.0 Hz, 1H), 3.23-3.21 (m, 1H), 3.0-3.07 (m, 1H), 2.87-2.70 (m, 2H), 2.61-2.39 (m, 3H), 2.18 (t, J=18.8 Hz, 3H), 2.02 (s, 3H), 2.00-1.82 (m, 4H)

### General Procedure for Preparation of Scheme 2, Intermediate 9B:

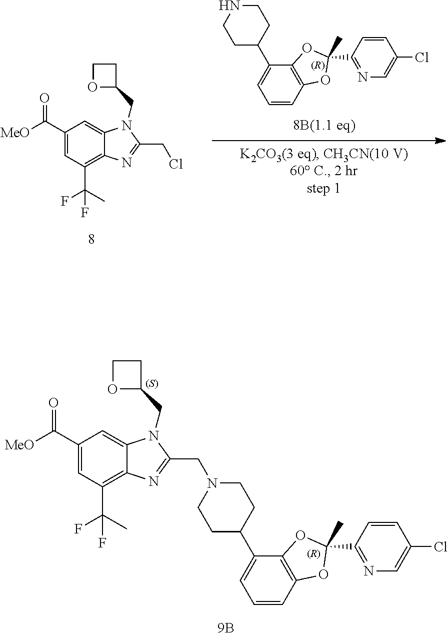

A mixture of 8 (120 mg, 334.48 μmol, 1 eq), 8B (121.71 mg, 367.93 μmol, 1.1 eq), K₂CO₃ (138.68 mg, 1.00 mmol, 3 eq), in CH₃CN (3 mL) was degassed and purged with N₂ for 3 times, and then the mixture was stirred at 60° C. for 2 hr under N₂ atmosphere. LCMS (product: RT=2.402 min) showed the starting material was consumed completely. The reaction mixture was added H₂O (5 mL) and extracted with ethyl acetate (5 mL*4). The combined organic phase was washed with brine (8 mL), dried with anhydrous Na₂SO₄, filtered and concentrated in vacuum. The residue was purified by column chromatography (SiO₂, Petroleum ether/Ethyl acetate=100/1 to 0/1). 9B (170 mg, crude) was obtained as a white solid.

LCMS: RT=2.421 min, MS cal.: 652.2, [M+H]⁺=653.2

### General Procedure for Preparation of Scheme 2, Compound 71 (10B):

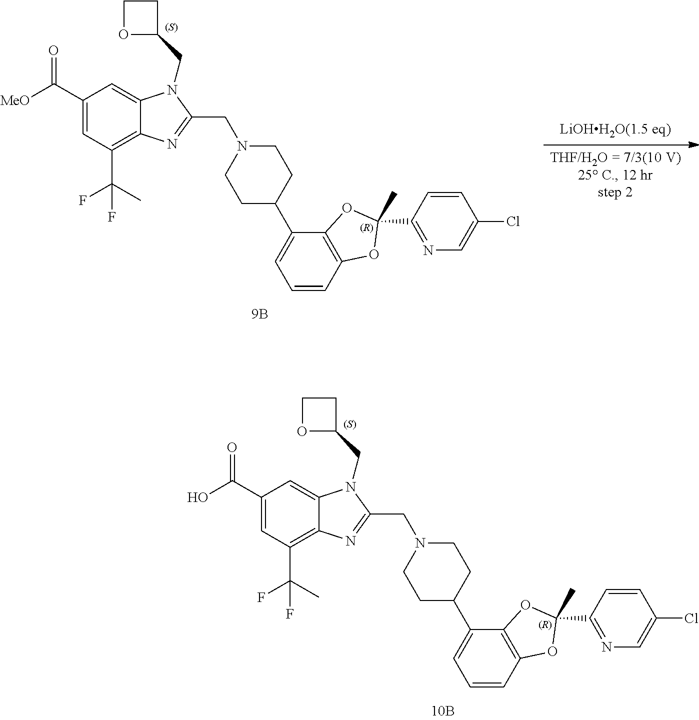

To a solution of 9B (150 mg, 229.67 μmol, 1 eq) in THF (1.4 mL) was added LiOH·H₂O (14.46 mg, 344.50 μmol, 1.5 eq) in H₂O (0.6 mL) at 20° C. The mixture was stirred at 25° C. for 12 hr. LCMS (product: RT=1.578 min) showed 9B was consumed completely. The reaction mixture was concentrated under reduced pressure to remove solvent. The residue was purified by prep-HPLC (column: Waters Xbridge Prep OBD C18 150*40 mm*10 um; mobile phase: [H₂O (10 mM NH₄HCO₃)—CH₃CN]; gradient: 20%-50% B over 8.0 min). 2-((4-((R)-2-(5-Chloropyridin-2-yl)-2-methylbenzo[d][1,3]dioxol-4-yl)piperidin-1-yl)methyl)-4-(1,1-difluoroethyl)-1-(((S)-oxetan-2-yl)methyl)-1H-benzo[d]imidazole-6-carboxylic acid (31.05 mg, 48.58 μmol, 21% yield) was obtained as a white solid.

LCMS: RT=2.770 min, MS cal.: 638.2, [M+H]⁺=639.0

HPLC: RT=11.795 min

SFC: RT=5.021 min

¹H NMR (400 MHz, METHANOL-d4) δ=8.61 (s, 1H), 8.41 (s, 1H), 8.12 (s, 1H), 7.88 (dd, J=2.4, 8.4 Hz, 1H), 7.65 (d, J=8.4 Hz, 1H), 6.83-6.69 (m, 3H), 5.32-5.22 (m, 1H), 4.88-4.86 (m, 1H), 4.75-4.73 (m, 1H), 4.69-4.60 (m, 1H), 4.53-4.44 (m, 1H), 4.21 (d, J=14.0 Hz, 1H), 4.11 (d, J=14.0 Hz, 1H), 3.26-3.18 (m, 1H), 3.15-3.03 (m, 1H), 2.87-2.73 (m, 2H), 2.61-2.41 (m, 3H), 2.19 (t, J=18.4 Hz, 3H), 2.02 (s, 3H), 1.98-1.79 (m, 4H)

### Example A277—Preparation of Compound 76 and Compound 309. (S)-2-((4-(2-(5-Chloropyridin-2-yl)-2-methylbenzo[d][1,3]dioxol-4-yl)piperidin-1-yl)methyl)-7-(2-methoxyethoxy)-1-methyl-1H-benzo[d]imidazole-5-carboxylic acid (Compound 76) (8) & (S)-2-((4-(2-(5-Chloropyridin-2-yl)-2-methylbenzo[d][1,3]dioxol-4-yl)piperidin-1-yl)methyl)-4-(2-methoxyethoxy)-1-methyl-1H-benzo[d]imidazole-6-carboxylic acid (Compound 309) (8A)

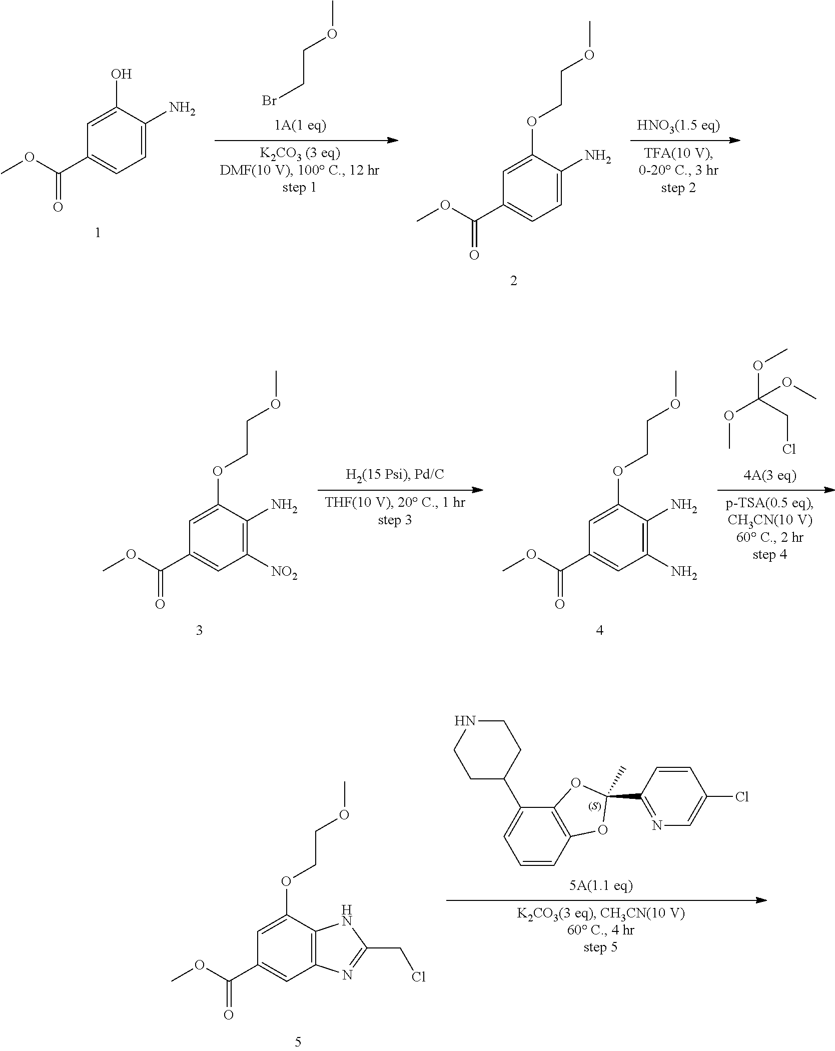

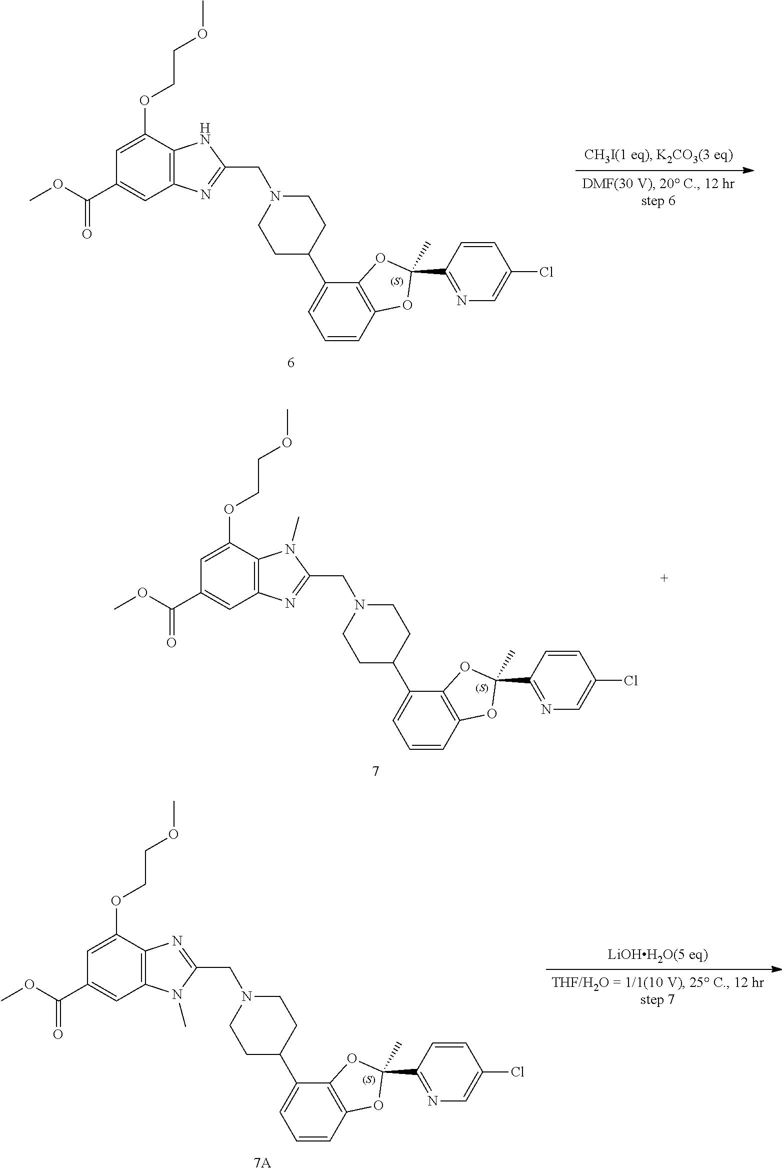

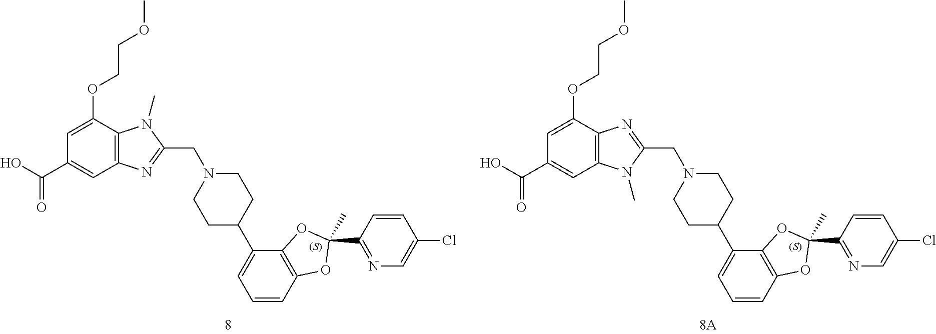

### General Procedure for Preparation of Intermediate 2:

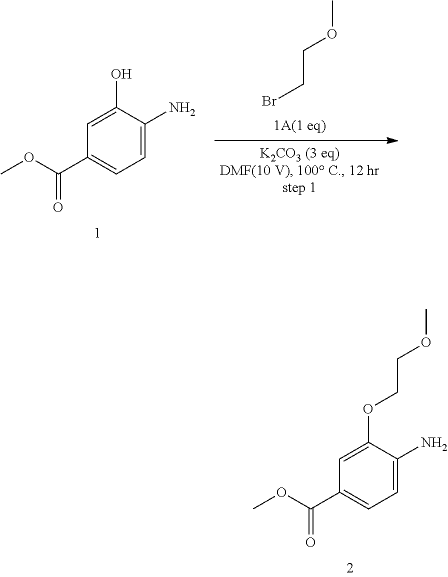

A mixture of 1 (5 g, 29.91 mmol, 1 eq), 1A (4.16 g, 29.91 mmol, 2.81 mL, 1 eq), K₂CO₃ (12.40 g, 89.73 mmol, 3 eq) in DMF (50 mL) was degassed and purged with N₂ for 3 times, and then the mixture was stirred at 100° C. for 12 hr under N₂ atmosphere. LCMS (product: RT=1.111 min) showed the 1 was consumed completely. The reaction mixture was diluted with H₂O (60 mL) and extracted with EtOAc (100 mL*3). The combined organic layers were washed with brine (50 mL*3), dried over Na₂SO₄, filtered and concentrated under reduced pressure to give a residue. The residue was purified by column chromatography (SiO₂, Petroleum ether/Ethyl acetate=100/1 to 1/1). 2 (4.81 g, 21.35 mmol, 71% yield) was obtained as a black solid.

LCMS: RT=1.111 min, MS cal.: 225.2, [M+H]⁺=226.2

¹H NMR (400 MHz, DMSO-d₆) δ=7.39 (dd, J=1.6, 8.4 Hz, 1H), 7.31 (s, 1H), 6.66 (d, J=8.4 Hz, 1H), 5.64-5.52 (m, 2H), 4.12-4.07 (m, 2H), 3.77-3.73 (m, 3H), 3.71-3.66 (m, 2H), 3.32 (s, 3H)

### General Procedure for Preparation of Intermediate 3:

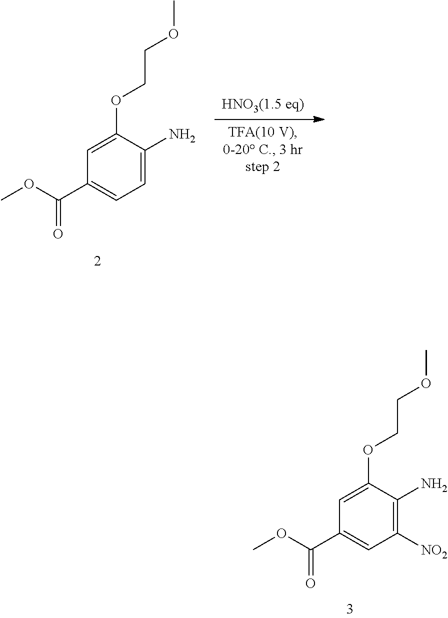

Equip a 250 mL three-necked round bottom flask and thermometer, N₂ balloon. TFA (38 mL) was charged to the 250 mL three-necked round bottom flask, then 2 (3.8 g, 16.87 mmol, 1 eq) was added at 20° C. At 0° C. inner temperature, KNO₃ (2.56 g, 25.31 mmol, 1.5 eq) was added in portions to the reaction mixture at 0° C. After the addition, the mixture was stirred at 0° C. for 3 hr. LCMS (product: RT=1.366 min) showed the 2 was consumed completely. After 3 hr, the reaction mixture was added dropwise to H₂O (60 mL) at 0° C. within 10 min. Then the reaction mixture was filtered, and the filter cake was dried in vacuum to give the crude product. The filtrate was extracted by EtOAc (80 mL*3). Then organic phase was combined and washed by brine (30 mL), dried with Na₂SO₄, filtered. The organic phase was concentrated under reduced pressure at 40° C. to give a residue. The residue was purified by column chromatography (SiO₂, Petroleum ether/Ethyl acetate=100/1 to 1/1). 3 (3.36 g, 12.43 mmol, 73.70% yield) was obtained as a yellow solid.

LCMS: RT=1.366 min, MS cal.: 270.2, [M+H]⁺=271.3

¹H NMR (400 MHz, DMSO-d₆) δ=8.27 (d, J=1.6 Hz, 1H), 7.56 (br s, 2H), 7.46 (d, J=1.6 Hz, 1H), 4.26 (dd, J=3.6, 5.2 Hz, 2H), 3.84 (s, 3H), 3.78-3.74 (m, 2H), 3.36-3.32 (m, 3H)

### General Procedure for Preparation of Intermediate 4:

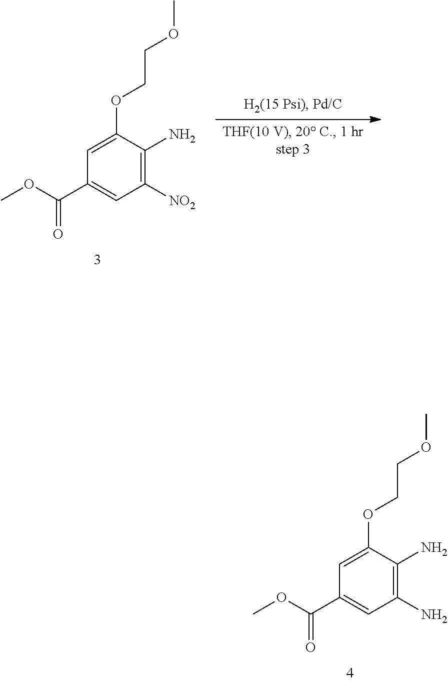 [SMILES: COCCOc1cc(C(=O)OC)cc([N+](=O)[O-])c1N ; "H2(15 Psi), Pd/C" ; "THF(10 V), 20° C., 1 hrstep 3" ; COCCOc1cc(C(=O)OC)cc(N)c1N]

THF (15 mL) was charged to the round bottom flask, then 3 (1 g, 3.70 mmol, 1 eq) and Pd/C (472.56 mg, 10% purity) was added to the reaction mixture at 20° C. under H₂ atmosphere. After the addition, the suspension was degassed and purged with H₂ for 3 times, and then it was stirred under H₂ atmosphere at 20° C. for 1 hr. LCMS (product: RT=1.095 min) showed compound 3 was consumed completely. The reaction mixture was diluted with THF (20 ml), and then filtered through celite pad. The filter caker was washed with THF (20 ml*3), and the filtrate was concentrated. 4 (889.05 mg, crude) was obtained as a yellow solid.

LCMS: RT=1.081 min, MS cal.: 240.3, [M+H]⁺=241.2

¹H NMR (400 MHz, DMSO-d₆) δ=6.98 (d, J=1.6 Hz, 1H), 6.85 (d, J=1.6 Hz, 1H), 4.82 (br s, 4H), 4.06 (dd, J=3.6, 5.2 Hz, 2H), 3.75-3.71 (m, 3H), 3.70-3.64 (m, 2H), 3.33 (s, 3H)

### General Procedure for Preparation of Intermediate 5:

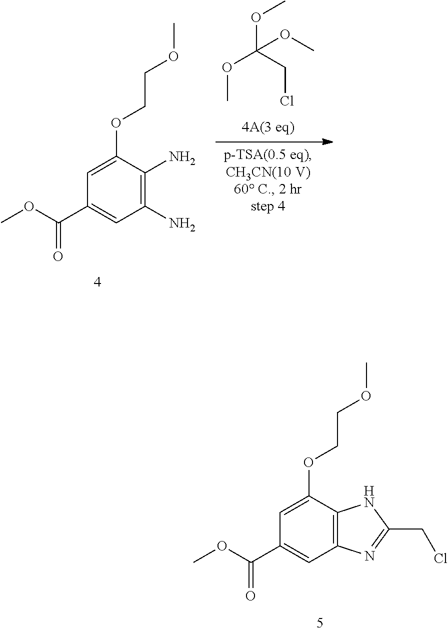

A mixture of 4 (880 mg, 3.66 mmol, 1 eq), 4A (1.70 g, 10.99 mmol, 1.48 mL, 3 eq), p-TSA (315.37 mg, 1.83 mmol, 0.5 eq) in ACN (10 mL) was degassed and purged with N₂ for 3 times, and then the mixture was stirred at 60° C. for 2 hr under N₂ atmosphere. LCMS (product: RT=1.325 min) showed the 4 was consumed completely. The reaction mixture was concentrated under reduced pressure to give a residue. 5 (1 g, crude) was obtained as a yellow oil.

LCMS: RT=1.325 min, MS cal.: 298.7, [M+H]⁺=299.1

### General Procedure for Preparation of Intermediate 6:

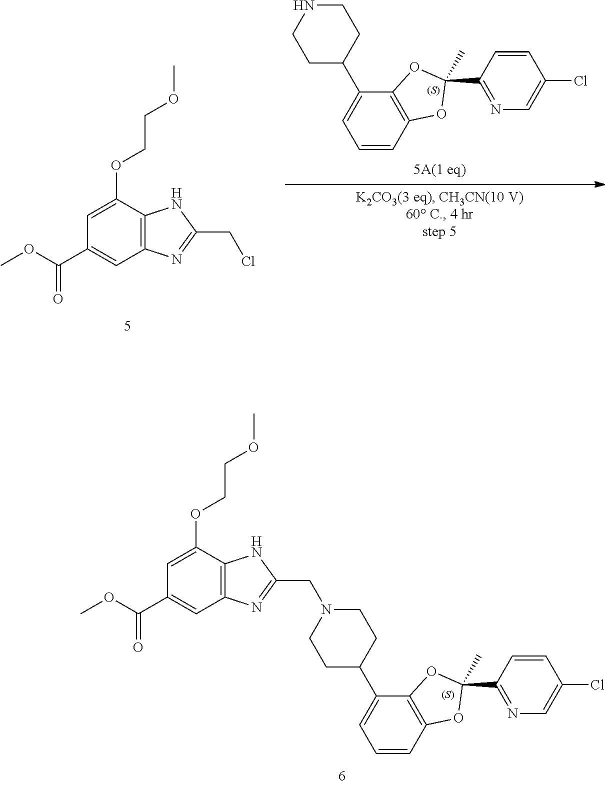

A mixture of 5 (1 g, 3.35 mmol, 1 eq), 5A (1.11 g, 3.35 mmol, 1 eq), K₂CO₃ (1.39 g, 10.04 mmol, 3 eq) in ACN (15 mL) was degassed and purged with N₂ for 3 times, and then the mixture was stirred at 60° C. for 4 hr under N₂ atmosphere. LCMS (product: RT=2.196 min) showed the 5 was consumed completely. The reaction mixture was diluted with H₂O (10 mL) and extracted with EtOAc (20 mL*3). The combined organic layers were washed with brine (10 mL*2), dried over Na₂SO₄, filtered and concentrated under reduced pressure to give a residue. The residue was purified by column chromatography (SiO₂, Ethyl acetate/MeOH=100/1 to 1/1). 6 (800 mg, 1.35 mmol, 40.30% yield) was obtained as a yellow solid.

LCMS: RT=2.194 min, MS cal.: 593.1, [M+H]⁺=593.2

¹H NMR (400 MHz, DMSO-d₆) δ=13.09-12.57 (m, 1H), 8.72 (d, J=2.4 Hz, 1H), 8.00 (dd, J=2.4, 8.4 Hz, 1H), 7.88-7.69 (m, 1H), 7.59 (d, J=8.4 Hz, 1H), 7.37-7.18 (m, 1H), 6.83-6.69 (m, 3H), 4.39-4.27 (m, 2H), 3.89-3.83 (m, 3H), 3.80-3.70 (m, 4H), 3.34 (s, 3H), 3.05-2.89 (m, 2H), 2.69-2.56 (m, 1H), 2.18 (br t, J=11.2 Hz, 2H), 1.99 (d, J=5.2 Hz, 4H), 1.88-1.64 (m, 4H)

### General Procedure for Preparation of Intermediate 7 & 7A:

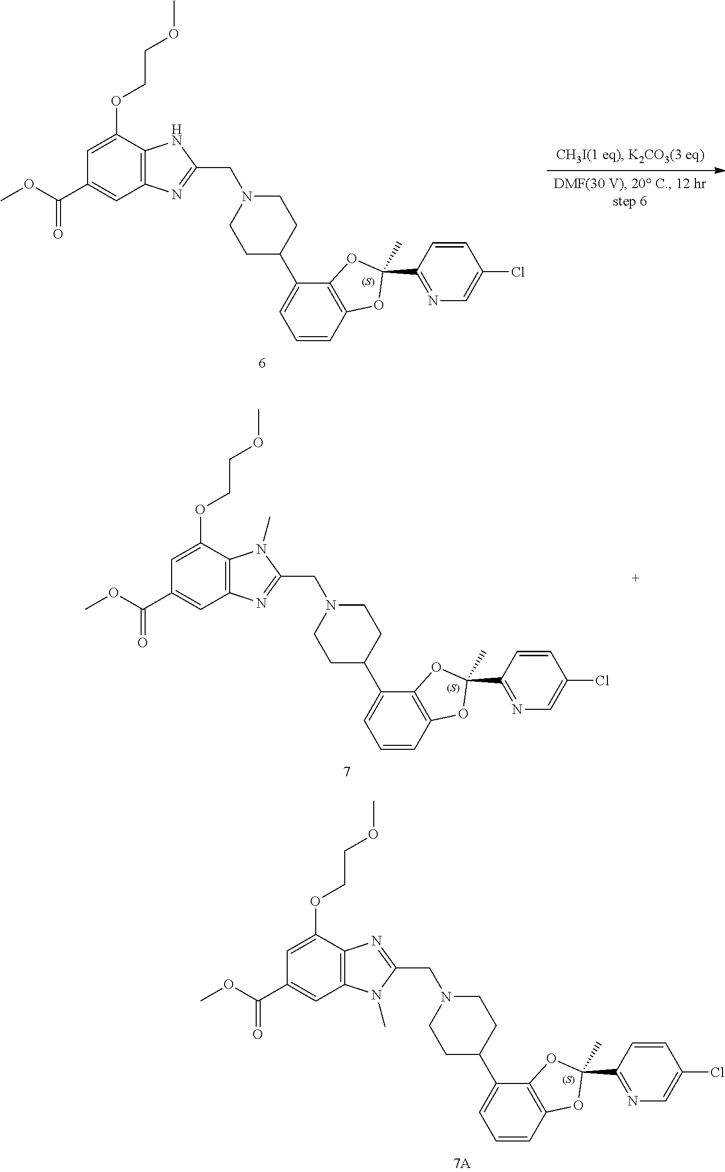

7 reactions were carried out in parallel. DMF (2 mL) was charged to the three-necked round bottom flask, then 6 (100 mg, 168.61 μmol, 1 eq) and K₂CO₃ (69.91 mg, 505.84 μmol, 3 eq) was added at 20° C. At 0° C., CH₃I (23.93 mg, 168.61 μmol, 10.50 μL, 1 eq) in DMF (1 mL) was added dropwise to the reaction mixture at 0° C. After the addition, the mixture was stirred at 20° C. for 12 hr. LCMS (RT₇=2.463 min, RT_{7A}=2.361 min) showed 6 was consumed completely and desired mass was detected. After 12 hr, the 7 reactions were combined for work up. The reaction mixture was added dropwise to H₂O (35 mL) at 0° C. The mixture was extracted by EtOAc (30 mL*3). Then organic phase was washed by brine (40 mL), dried with Na₂SO₄, filtered. The organic phase was concentrated under reduced pressure at 45° C. to give a residue. The crude product was purified by prep-HPLC (column: Waters Xbridge Prep OBD C18 150*40 mm*10 um; mobile phase: [H₂O (10 mM NH₄HCO₃)-ACN]; gradient: 50%-80% B over 8.0 min). 7 (85 mg, 140.01 μmol, 11.86% yield) was obtained as a white solid. 7A (75 mg, 123.54 μmol, 10.46% yield) was obtained as a white solid.

LCMS: RT₇=2.463 min, MS cal.: 607.1, [M+H]⁺=607.2

¹H NMR (400 MHz, CHLOROFORM-d) δ=8.62 (d, J=2.0 Hz, 1H), 8.10-8.07 (m, 1H), 7.68 (dd, J=2.4, 8.4 Hz, 1H), 7.58 (br d, J=8.4 Hz, 1H), 7.40 (s, 1H), 6.79 (dd, J=6.8, 8.8 Hz, 1H), 6.71 (d, J=7.6 Hz, 2H), 4.36-4.30 (m, 2H), 4.21 (s, 3H), 3.93 (s, 3H), 3.90-3.75 (m, 4H), 3.48 (s, 3H), 3.01 (br s, 2H), 2.77 (br s, 1H), 2.47-2.19 (m, 2H), 2.05 (s, 3H), 1.96-1.75 (m, 4H)

LCMS: RT_{7A}=2.361 min, MS cal.: 607.1, [M+H]⁺=607.2

¹H NMR (400 MHz, CHLOROFORM-d) δ=8.62 (d, J=1.6 Hz, 1H), 7.78 (s, 1H), 7.69 (dd, J=1.6, 8.4 Hz, 1H), 7.60 (br s, 1H), 7.44 (s, 1H), 6.82-6.75 (m, 1H), 6.76-6.68 (m, 2H), 4.45 (t, J=5.2 Hz, 2H), 3.96 (s, 6H), 3.91 (br t, J=5.2 Hz, 4H), 3.47 (s, 3H), 2.97 (br s, 2H), 2.86-2.70 (m, 1H), 2.43-2.20 (m, 2H), 2.05 (s, 3H), 1.93-1.68 (m, 4H)

### General Procedure for Preparation of Compound 76 (8):

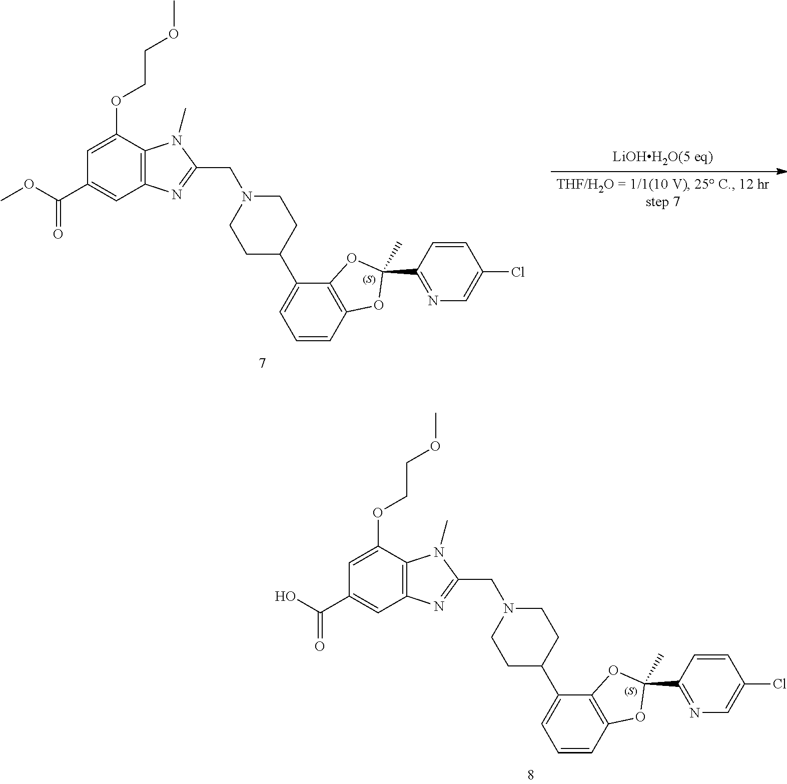

A mixture of 7 (70 mg, 115.30 μmol, 1 eq), LiOH·H₂O (24.19 mg, 576.52 μmol, 5 eq) in THF (0.5 mL) and H₂O (0.5 mL) was degassed and purged with N₂ for 3 times, and then the mixture was stirred at 25° C. for 12 hr under N₂ atmosphere. LCMS (RT=1.549 min) showed 7 was consumed completely and desired mass was detected. The crude product was purified by prep-HPLC (column: Waters Xbridge Prep OBD C18 150*40 mm*10 um; mobile phase: [H₂O (10 mM NH₄HCO₃)-ACN]; gradient: 20%-60% B over 8.0 min). (S)-2-((4-(2-(5-Chloropyridin-2-yl)-2-methylbenzo[d][1,3]dioxol-4-yl)piperidin-1-yl)methyl)-7-(2-methoxyethoxy)-1-methyl-1H-benzo[d]imidazole-5-carboxylic acid (28.51 mg, 48.00 μmol, 41.63% yield, 99.85% purity) was obtained as a white solid.

LCMS: RT=2.715 min, MS cal.: 593.08, [M+H]⁺=593.0

HPLC: RT=10.074 min, purity: 99.89%

¹H NMR (400 MHz, DMSO-d₆) δ=8.72 (d, J=2.4 Hz, 1H), 8.00 (dd, J=2.4, 8.4 Hz, 1H), 7.80 (d, J=1.2 Hz, 1H), 7.59 (d, J=8.4 Hz, 1H), 7.31 (s, 1H), 6.82-6.72 (m, 3H), 4.35-4.22 (m, 2H), 4.10 (s, 3H), 3.80-3.73 (m, 4H), 3.36-3.34 (m, 3H), 2.92 (br s, 2H), 2.69-2.59 (m, 1H), 2.25-2.11 (m, 2H), 2.00 (s, 3H), 1.72 (br d, J=9.6 Hz, 4H)

### General Procedure for Preparation of Compound 309 (8A):

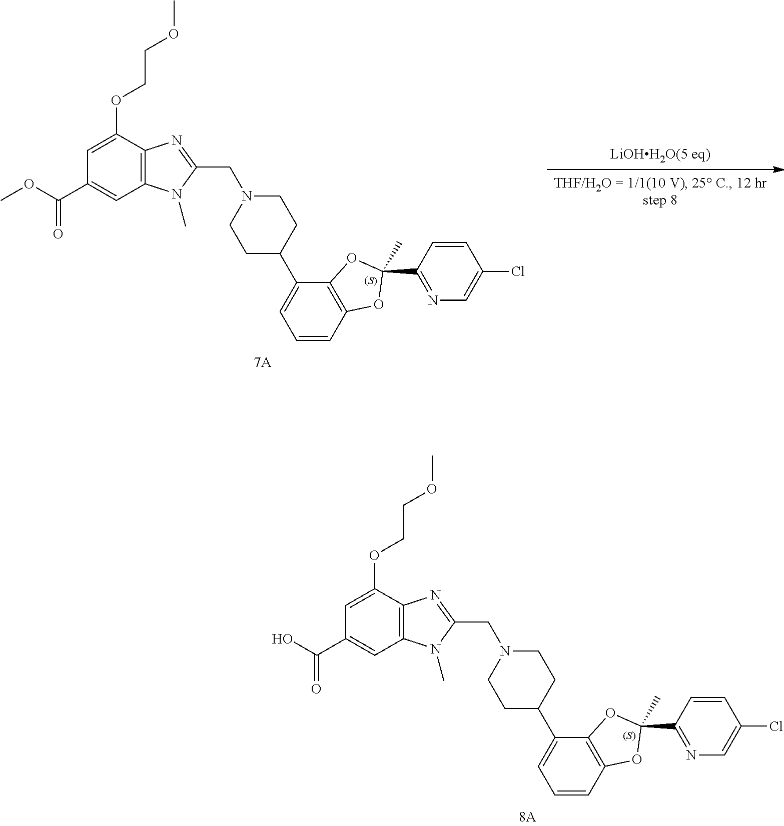

A mixture of 7A (70 mg, 115.30 μmol, 1 eq), LiOH·H₂O (24.19 mg, 576.52 μmol, 5 eq) in THF (0.5 mL) and H₂O (0.5 mL) was degassed and purged with N₂ for 3 times, and then the mixture was stirred at 25° C. for 12 hr under N₂ atmosphere. LCMS (product: RT=1.417 min) showed the 7A was consumed completely. The crude product was purified by reversed-phase HPLC (column: Waters Xbridge Prep OBD C18 150*40 mm*10 um; mobile phase: [H₂O (10 mM NH₄HCO₃)-ACN]; gradient: 25%-55% B over 8.0 min). 309 (28.77 mg, 48.51 μmol, 42.07% yield) was obtained as a white solid.

LCMS: RT=2.576 min, MS cal.: 593.1, [M+H]⁺=593.0

HPLC: RT=10.532 min, purity=99.85%

¹H NMR (400 MHz, METHANOL-d₄) δ=8.60 (d, J=2.4 Hz, 1H), 7.90-7.85 (m, 2H), 7.65 (d, J=8.4 Hz, 1H), 7.45 (d, J=0.8 Hz, 1H), 6.84-6.67 (m, 3H), 4.37 (dd, J=3.6, 5.2 Hz, 2H), 3.97 (s, 3H), 3.93 (s, 2H), 3.88 (dd, J=3.6, 5.2 Hz, 2H), 3.45 (s, 3H), 3.07 (br dd, J=2.8, 11.2 Hz, 2H), 2.80-2.69 (m, 1H), 2.44-2.30 (m, 2H), 2.01 (s, 3H), 1.92-1.80 (m, 4H)

### Example A278—Preparation of Compound 77. 2-((4-((S)-2-(5-chloropyridin-2-yl)-2-methylbenzo[d][1,3]dioxol-4-yl)piperidin-1-yl)methyl)-1-(((S)-oxetan-2-yl)methyl)-4-(oxetan-3-ylmethoxy)-1H-benzo[d]imidazole-6-carboxylic acid (Compound 77) (8)

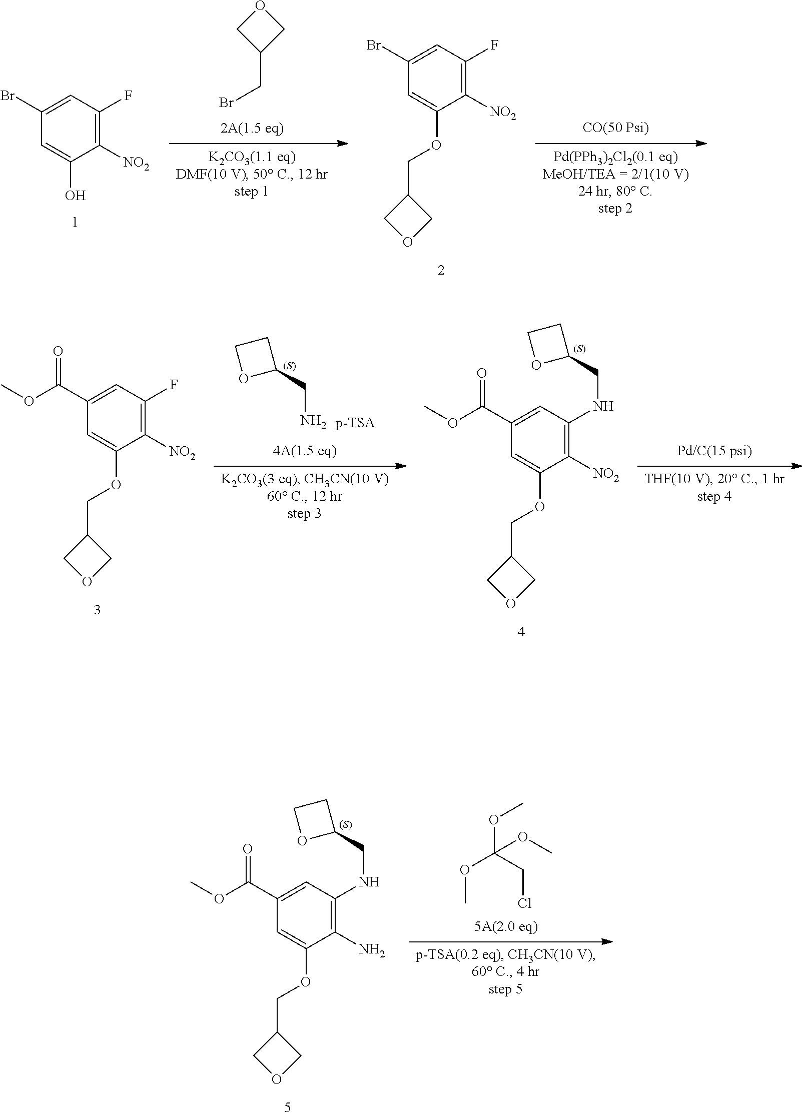

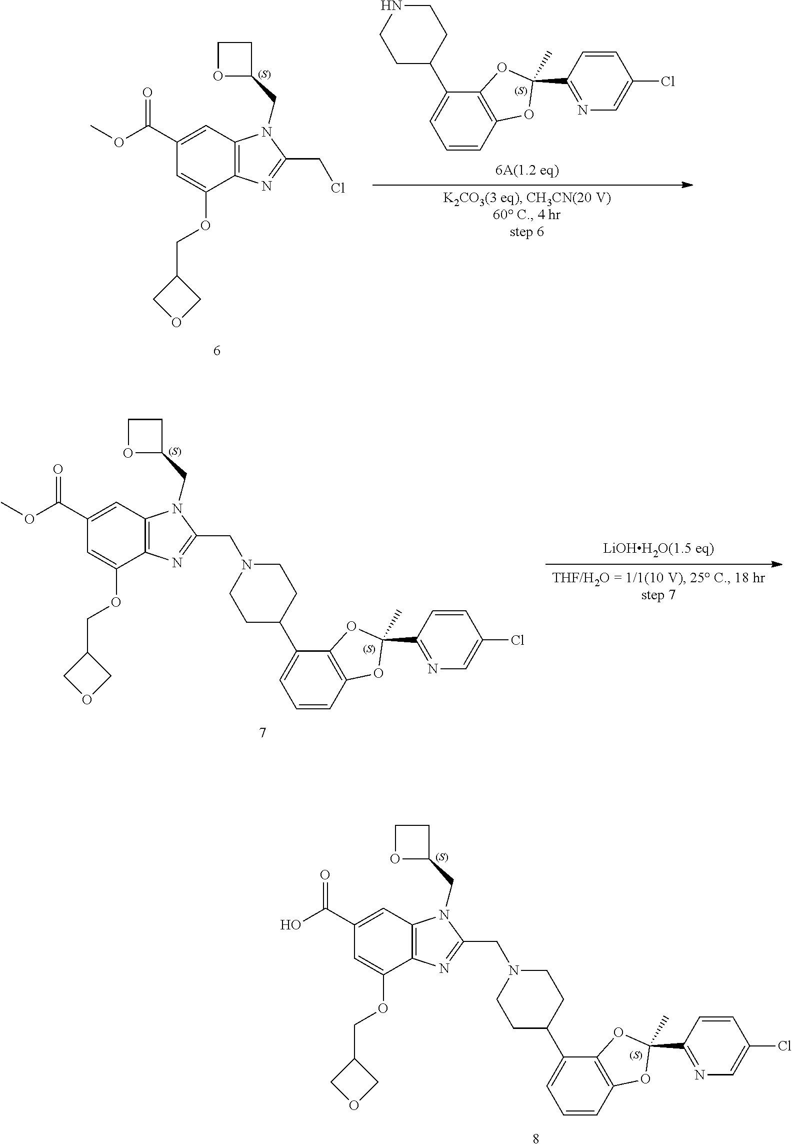

### General Procedure for Preparation of Intermediate 2:

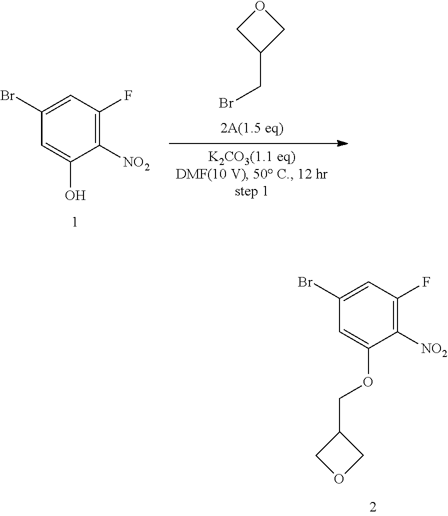

To a solution of 1 (3 g, 12.71 mmol, 1 eq) in DMF (30 mL) was added K₂CO₃ (1.93 g, 13.98 mmol, 1.1 eq) and 2A (2.88 g, 19.07 mmol, 1.5 eq). The mixture was stirred at 50° C. for 12 hr. LCMS showed it was finished. The reaction was poured into H₂O (50 ml). The mixture was extracted by EtOAc (50 mL*3). Then organic phase was combined and washed by H₂O (50 mL), brine (50 mL), dried with Na₂SO₄, filtered. The organic phase was concentrated under reduced pressure at 45° C. to give a residue. The residue was purified by column chromatography (SiO₂, PE/EtOAc=10/1 to 1/1). 2 (3.4 g, 11.11 mmol, 87% yield) was obtained as a brown solid.

LCMS: RT=0.479 min, MS cal.: 304.97, [M+H]⁺=306.0

### General Procedure for Preparation of Intermediate 3:

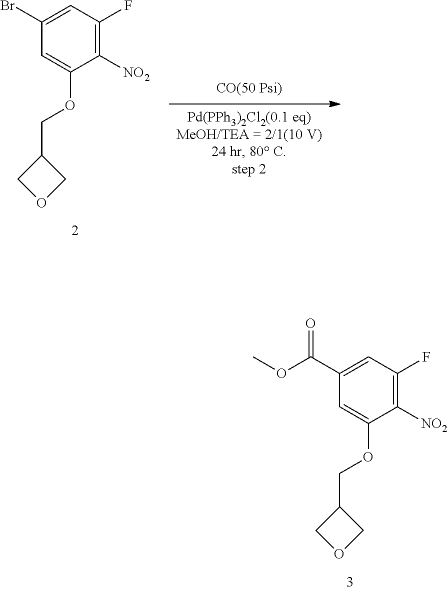

MeOH (25 mL) and TEA (12 mL) was charged to the 40 mL Hydrogenated bottle, then 2 (3.3 g, 10.78 mmol, 1 eq) and Pd(PPh₃)₂Cl₂ (756.74 mg, 1.08 mmol, 0.1 eq) was added. After the addition, the mixture was degassed and purged with CO for 3 times. The mixture was stirred at 80° C. under CO (50 psi) for 23.5 hr. TLC (PE/EtOAc=2/1 Rf=0.5) indicated 2 was consumed completely. The reaction mixture was diluted with H₂O (50 mL) and adjusted pH to 7-8 with 1M FA, then it was extracted with EtOAc (20 mL*3). The combined organic layers were washed with brine (50 ml*2), dried over Na₂SO₄, filtered, and concentrated under reduced pressure to give a crude product. The crude product was triturated with PE (20 mL) at 20° C. for 20 min. The mixture was filtered, and the filter cake was dried under vacuum at 45° C. for 30 mins. 3 (2.6 g, 9.12 mmol, 84% yield) was obtained as a yellow solid.

LCMS: RT=0.440 min, MS cal.: 285.06, [M+H]⁺=286.2

¹H NMR (400 MHz, CHLOROFORM-d) δ=7.58-7.52 (m, 2H), 4.93-4.84 (m, 2H), 4.54-4.48 (m, 2H), 4.42 (d, J=6.8 Hz, 2H), 3.98 (s, 3H), 3.51-3.41 (m, 1H)

### General Procedure for Preparation of Intermediate 4:

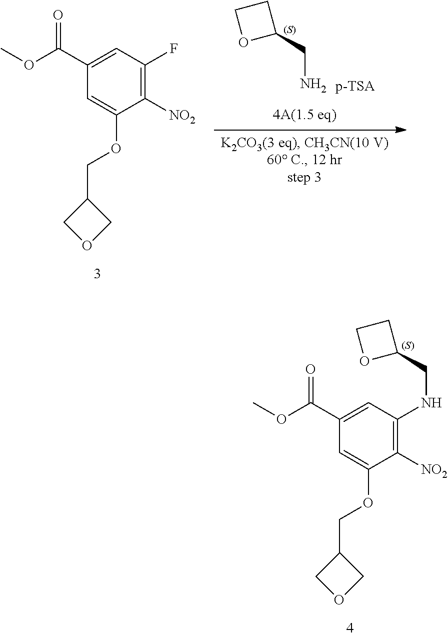

To a solution of 3 (700 mg, 2.45 mmol, 1 eq) in CH₃CN (7 mL) was added K₂CO₃ (1.02 g, 7.36 mmol, 3 eq) and 4A (954.64 mg, 3.68 mmol, 1.5 eq, p-TSA) at 25° C. The mixture was stirred at 60° C. for 12 hr. LC-MS showed 3 was consumed completely and one main peak with desired mass was detected. The mixture was diluted with H₂O (7 mL), extracted with EtOAc (7 mL*3). The combined organic layer was washed with H₂O (7 mL), brine (7 mL), dried over Na₂SO₄, filtered and the filtrate was concentrated to give a crude product. The residue was purified by column chromatography (SiO₂, PE/EtOAc=10/1 to 0/1). 4 (470 mg, 1.33 mmol, 54% yield) was obtained as a white solid.

LCMS: RT=0.408 min, MS cal.: 352.13, [M+H]⁺=353.0

¹H NMR (400 MHz, CHLOROFORM-d) δ=7.17 (d, J=1.6 Hz, 1H), 6.98 (d, J=2.0 Hz, 1H), 6.21-6.11 (m, 1H), 5.13-5.05 (m, 1H), 4.88 (dd, J=6.4, 1.2 Hz, 1H), 4.73 (dt, J=6.0, 7.6 Hz, 1H), 4.61-4.53 (m, 3H), 4.36 (d, J=6.8 Hz, 2H), 3.95 (s, 3H), 3.56-3.42 (m, 3H), 2.77-2.69 (m, 1H), 2.61-2.52 (m, 1H)

### General Procedure for Preparation of Intermediate 5:

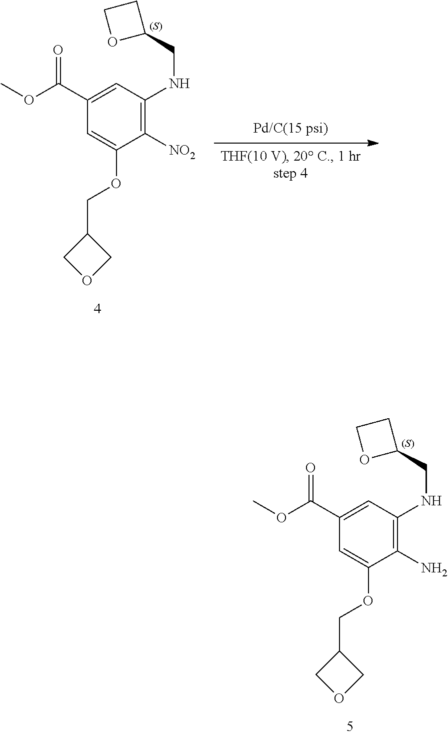

To a solution of 4 (660 mg, 1.87 mmol, 1 eq) in THF (7 mL) was added Pd/C (660.00 mg, 10% purity) under N₂ atmosphere. The suspension was degassed and purged with H₂ for 3 times. The mixture was stirred under H₂ (15 Psi) at 20° C. for 1 hr. LC-MS showed 4 was consumed completely and one main peak with desired mass was detected. The suspension was filtered, and the filter cake was washed with THF (5 mL×3). The combined filtrates were concentrated to give 5 (800 mg, crude) as a yellow oil.

LCMS: RT=0.331 min, MS cal.: 322.15, [M+H]⁺=323.2

¹H NMR (400 MHz, CHLOROFORM-d) δ=7.20 (d, J=1.2 Hz, 1H), 7.17 (d, J=1.6 Hz, 1H), 5.31 (s, 1H), 5.14-5.06 (m, 1H), 4.91 (dd, J=6.4, 1.2 Hz, 2H), 4.78-4.71 (m, 1H), 4.65-4.58 (m, 3H), 4.32-4.28 (m, 2H), 3.88 (s, 3H), 3.51-3.42 (m, 2H), 3.39 (d, J=3.6 Hz, 1H), 2.80-2.70 (m, 1H), 2.64-2.53 (m, 1H).

### General Procedure for Preparation of Intermediate 6:

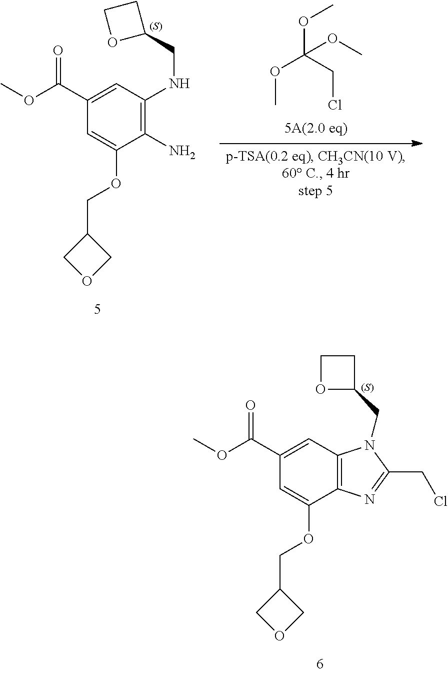

To a solution of 5 (350 mg, 1.09 mmol, 1 eq) in CH₃CN (5 mL) was added 5A (335.70 mg, 2.17 mmol, 292.68 μL, 2 eq) and p-TSA (37.39 mg, 217.15 μmol, 0.2 eq). The mixture was stirred at 60° C. for 2 hr. LC-MS showed 5 was consumed completely and one main peak with desired mass was detected. The mixture was diluted with H₂O (5 mL), extracted with EtOAc (5 mL*3). The combined organic layer was washed with H₂O (5 mL), brine (5 mL), dried over Na₂SO₄, filtered and the filtrate was concentrated to give a crude product. The residue was purified by column chromatography (SiO₂, PE/EtOAc=10/1 to 0/1). 6 (230 mg, 603.96 μmol, 55% yield) was obtained as a yellow solid.

LCMS: RT=0.373 min, MS cal.: 380.11, [M+H]⁺=381.1.

¹H NMR (400 MHz, CHLOROFORM-d) δ=7.78 (s, 1H), 7.46 (s, 1H), 5.21 (br dd, J=2.4, 5.6 Hz, 1H), 5.10-4.93 (m, 4H), 4.66-4.50 (m, 7H), 4.32 (td, J=5.6, 9.2 Hz, 1H), 3.97 (s, 3H), 3.72-3.57 (m, 1H), 2.81-2.69 (m, 1H), 2.46-2.35 (m, 1H)

### General Procedure for Preparation of Intermediate 7:

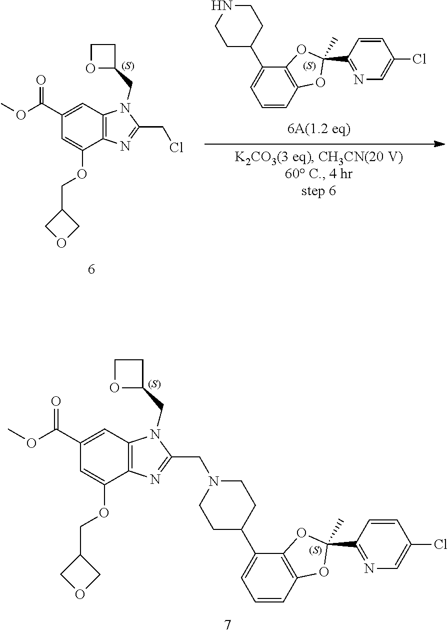

To a solution of 6 (200 mg, 525.18 μmol, 1 eq) and 6A (208.48 mg, 630.22 μmol, 1.2 eq)

(Synthesized from Int 5A) in CH₃CN (4 mL) was added K₂CO₃ (217.75 mg, 1.58 mmol, 3 eq) at 25° C. The mixture was stirred at 60° C. for 4 hr. LC-MS showed 6 was consumed completely and one main peak with desired mass was detected. The mixture was diluted with H₂O (2 mL), extracted with EtOAc (2 mL*3). The combined organic layer was washed with H₂O (2 mL), brine (2 mL), dried over Na₂SO₄, filtered and the filtrate was concentrated to give a crude product. 7 (265 mg, 392.49 μmol, 74% yield) was obtained as a yellow solid.

LCMS: RT=0.441 min, MS cal.: 674.25, [M+H]⁺=675.4.

¹H NMR (400 MHz, CHLOROFORM-d) δ=8.64-8.60 (m, 1H), 7.86-7.83 (m, 1H), 7.71-7.65 (m, 1H), 7.58-7.54 (m, 1H), 7.43 (s, 1H), 6.82-6.76 (m, 1H), 6.74-6.68 (m, 2H), 5.26-5.18 (m, 1H), 4.98-4.92 (m, 2H), 4.74 (br d, J=5.6 Hz, 2H), 4.61-4.55 (m, 4H), 4.43-4.36 (m, 1H), 3.97-3.95 (m, 3H), 3.69-3.61 (m, 1H), 3.00-2.88 (m, 2H), 2.79-2.68 (m, 2H), 2.51-2.40 (m, 1H), 2.37-2.23 (m, 2H), 2.05 (s, 3H), 1.92-1.73 (m, 5H), 1.25 (s, 1H), 0.09-0.07 (m, 1H).

### General Procedure for Preparation of Compound 77 (8):

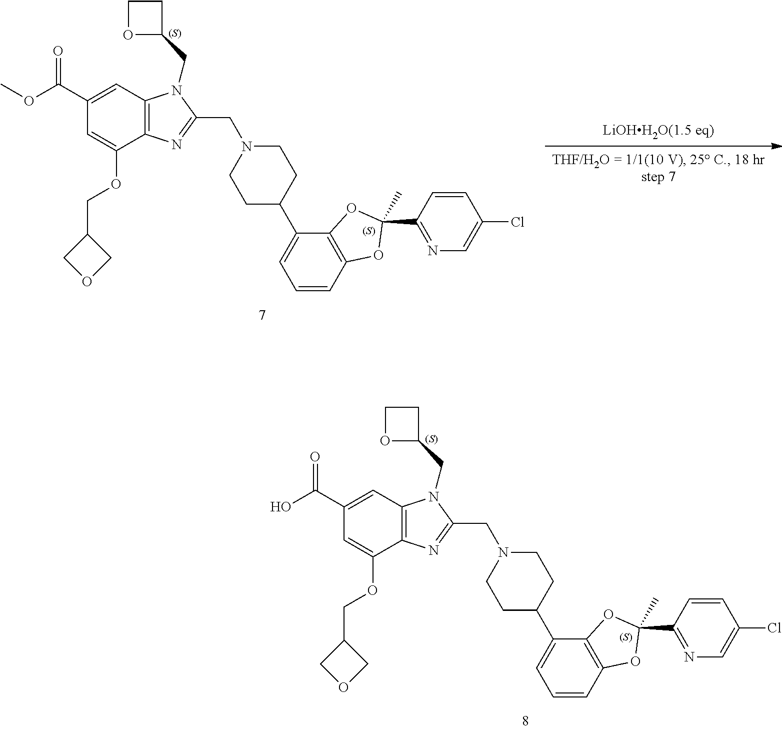

To a solution of 7 (100 mg, 148.11 μmol, 1 eq) in THF (1 mL) was added LiOH·H₂O (10.57 mg, 251.79 μmol, 1.7 eq) in H₂O (1 mL). The mixture was stirred at 25° C. for 18 hr. LC-MS showed 7 was consumed completely and one main peak with desired mass was detected. The reaction mixture was filtered directly. The residue was purified by prep-HPLC (column: Waters Xbridge Prep OBD C18 150*40 mm*10 um; mobile phase: [H₂O (10 mM NH₄HCO₃)-ACN]; gradient: 35%-65% B over 8.0 min). 2-((4-((S)-2-(5-chloropyridin-2-yl)-2-methylbenzo[d][1,3]dioxol-4-yl)piperidin-1-yl)methyl)-1-(((S)-oxetan-2-yl)methyl)-4-(oxetan-3-ylmethoxy)-1H-benzo[d]imidazole-6-carboxylic acid (24.78 mg, 37.81 μmol, 25.31% yield) was obtained as a white solid.

LCMS: RT=2.610 min, MS cal.: 660.24, [M+H]⁺=661.3

HPLC: Rt=10.259 min, purity: 100%

¹H NMR (400 MHz, DMSO-d₆) δ=13.10-12.50 (m, 1H), 8.73-8.70 (m, 1H), 8.03-7.98 (m, 1H), 7.92-7.89 (m, 1H), 7.61-7.57 (m, 1H), 7.32-7.28 (m, 1H), 6.82-6.73 (m, 3H), 5.12-5.04 (m, 1H), 4.78-4.70 (m, 3H), 4.65-4.57 (m, 1H), 4.52-4.43 (m, 5H), 4.38-4.30 (m, 1H), 3.94-3.87 (m, 1H), 3.78-3.72 (m, 1H), 3.52-3.44 (m, 1H), 3.01-2.94 (m, 1H), 2.85-2.79 (m, 1H), 2.67-2.59 (m, 2H), 2.44-2.37 (m, 1H), 2.26-2.10 (m, 2H), 2.04-1.97 (m, 3H), 1.82-1.65 (m, 4H)

### Example A279—Preparation of Compound 87. (S)-2-((4-(2-(5-Chloropyridin-2-yl)-2-methylbenzo[d][1,3]dioxol-4-yl)piperidin-1-yl)methyl)-4-(1,1-difluoroethyl)-1-methyl-1H-benzo[d]imidazole-6-carboxylic acid (Compound 87) (7)

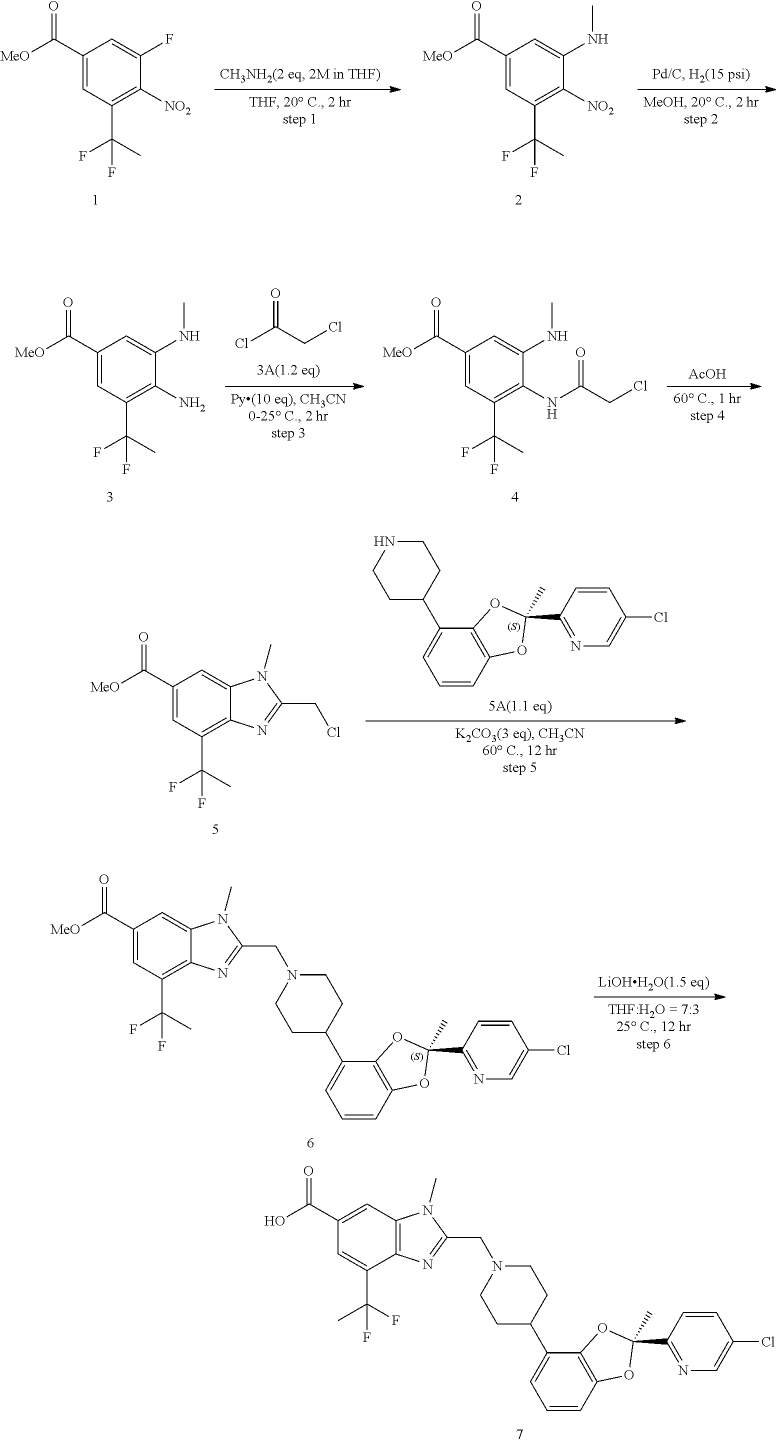

### General Procedure for Preparation of Intermediate 2:

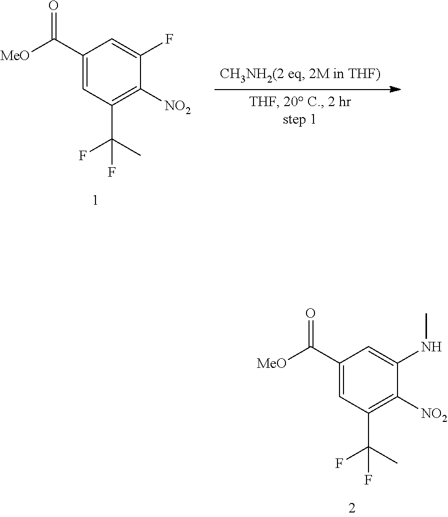 [SMILES: COC(=O)c1cc(F)c([N+](=O)[O-])c(C(C)(F)F)c1 ; "CH3NH2(2 eq, 2M in THF)" ; "THF, 20° C., 2 hrstep 1" ; CNc1cc(C(=O)OC)cc(C(C)(F)F)c1[N+](=O)[O-]]

Equip a 100 mL three-necked round bottom flask, and thermometer, N₂ balloon. THF (10 mL) was charged to the three-necked round bottom flask, then 1 (1 g, 3.80 mmol, 1 eq) was added. CH₃NH₂ (3.80 mL, 2 eq, 2 M in THF) was added dropwise to the reaction mixture at 20° C. within 10 min. After the addition, the mixture was stirred at 20° C. for 2 hr. The reaction was monitored by TLC. TLC (Petroleum ether/Ethyl acetate=10/1, R_{f}=0.20) indicated 1 was consumed completely and one new spot formed. The reaction was clean according to TLC. The reaction mixture was added FA (1 M) to adjust pH=4 at 20° C. The mixture was extracted by EtOAc 150 mL (50 mL*3). Then organic phase was combined and washed by H₂O 50 mL, brine 30 mL, dried with Na₂SO₄, filtered. The organic phase was concentrated under reduced pressure at 40° C. to give a residue. The residue was purified by column chromatography (SiO₂, Petroleum ether/Ethyl acetate=100/1 to 20/1). The residue was repurified by prep-HPLC (neutral condition; column: Phenomenex Gemini-NX 80*40 mm*3 um; mobile phase: [H₂O (10 mM NH₄HCO₃)-ACN]; gradient: 25%-50% B over 8.0 min). 2 (300 mg, 1.09 mmol, 29% yield) was obtained as a red solid.

¹H NMR (400 MHz, CHLOROFORM-d) δ=7.53 (s, 1H), 7.50 (d, J=1.6 Hz, 1H), 5.06 (s, 1H), 3.97 (s, 3H), 2.97 (s, 3H), 2.10 (t, J=18.4 Hz, 3H)

### General Procedure for Preparation of Intermediate 3:

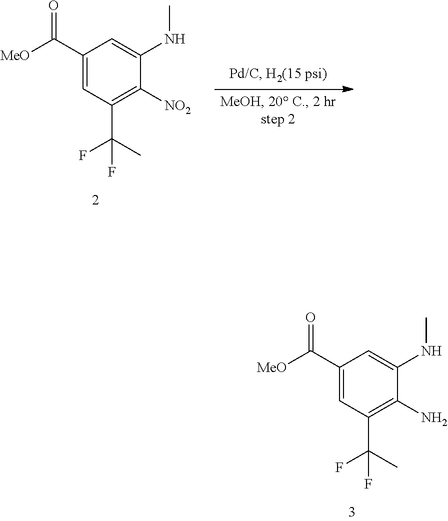 [SMILES: CNc1cc(C(=O)OC)cc(C(C)(F)F)c1[N+](=O)[O-] ; "MeOH, 20° C., 2 hrstep 2" ; "Pd/C, H2(15 psi)" ; CNc1cc(C(=O)OC)cc(C(C)(F)F)c1N]

MeOH (1 mL) was charged to a 100 mL round bottom flask, then 2 (200 mg, 729.34 μmol, 1 eq) was added. After the addition, the mixture was added Pd/C (50 mg) and MeOH (1 mL) degassed and purged with H₂ for 3 times. The mixture was stirred at 20° C. for 2 hr under H₂ atmosphere. The reaction was monitored by TLC. TLC (Petroleum ether/Ethyl acetate=10/1, R_{f}=0.17) indicated 2 was consumed completely and one new spot formed. The reaction was clean according to TLC. The reaction mixture was filtered by kieselguhr. The filter cake was washed with MeOH (30 mL). The filtrate was concentrated under reduced pressure to give a residue. The residue was purified by column chromatography (SiO₂, Petroleum ether/Ethyl acetate=100/1 to 10/1). 3 (110 mg, 450.38 μmol, 62% yield) was obtained as a yellow oil.

### General Procedure for Preparation of Intermediate 4:

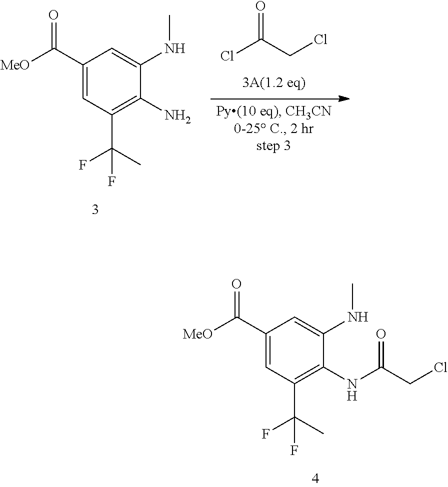 [SMILES: CNc1cc(C(=O)OC)cc(C(C)(F)F)c1N ; O=C(Cl)CCl ; "Py·(10 eq), CH3CN0-25° C., 2 hrstep 3" ; CNc1cc(C(=O)OC)cc(C(C)(F)F)c1NC(=O)CCl]

To a solution of 3 (110 mg, 450.38 μmol, 1 eq) in ACN (1 mL) was added Py. (356.25 mg, 4.50 mmol, 363.52 μL, 10 eq) at 25° C. Then the mixture was cooled to 0° C. and 3A (61.04 mg, 540.46 μmol, 43.05 μL, 1.2 eq) was added. The mixture was stirred at 25° C. for 2 hr. TLC (Petroleum ether/Ethyl acetate=2/1, R_{f}=0.33) indicated 3 was consumed completely and one new spot formed. The reaction was clean according to TLC. The reaction mixture was partitioned between H₂O 10 mL and DCM 30 mL. The organic phase was separated, washed with brine 10 mL, dried over Na₂SO₄, filtered and concentrated under reduced pressure to give the product. 4 (140 mg, 436.52 μmol, 97% yield) was obtained as a yellow solid.

¹H NMR (400 MHz, CHLOROFORM-d) δ=8.06 (s, 1H), 7.84 (s, 1H), 3.90 (s, 3H), 3.87 (s, 2H), 3.26 (s, 3H), 2.05 (t, J=18.8 Hz, 3H)

### General Procedure for Preparation of Intermediate 5:

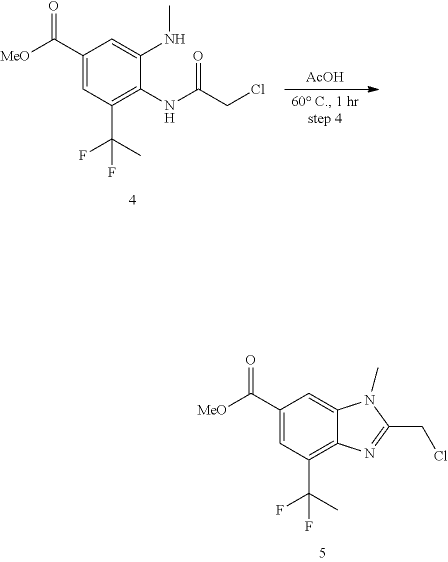 [SMILES: "AcOH" ; CNc1cc(C(=O)OC)cc(C(C)(F)F)c1NC(=O)CCl ; "60° C., 1 hrstep 4" ; COC(=O)c1cc(C(C)(F)F)c2nc(CCl)n(C)c2c1]

A mixture of 4 (140 mg, 436.52 μmol, 1 eq) in AcOH (1 mL) was degassed and purged with N₂ for 3 times, and then the mixture was stirred at 60° C. for 1 hr under N₂ atmosphere. TLC (Petroleum ether/Ethyl acetate=2/1, R_{f}=0.60) indicated 4 was consumed completely and one new spot formed. The reaction was clean according to TLC. The reaction mixture was concentrated under reduced pressure to give the product. 5 (130 mg, 429.46 μmol, 98% yield) was obtained as a yellow oil.

¹H NMR (400 MHz, CHLOROFORM-d) δ=8.21 (d, J=4.4 Hz, 2H), 4.92 (s, 2H), 3.99 (s, 3H), 3.97 (s, 3H), 2.24 (t, J=18.8 Hz, 3H)

### General Procedure for Preparation of Intermediate 6:

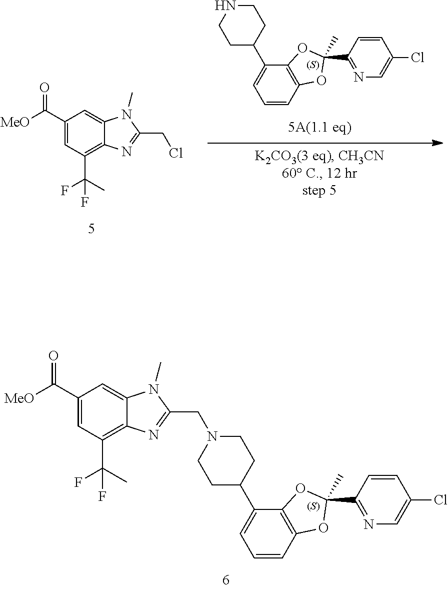

To a solution of 5 (130 mg, 429.46 μmol, 1 eq) in ACN (2 mL) was added K₂CO₃ (178.06 mg, 1.29 mmol, 3 eq) and 5A (156.28 mg, 472.41 μmol, 1.1 eq). The mixture was stirred at 60° C. for 12 hr. TLC (Petroleum ether/Ethyl acetate=1/1, R_{f}=0.30) indicated 5 was consumed completely and one new spot formed. The reaction was clean according to TLC. The reaction mixture was partitioned between H₂O 10 mL and EtOAc 30 mL. The organic phase was separated, washed with brine 10 mL, dried over Na₂SO₄, filtered and concentrated under reduced pressure to give a residue. The residue was purified by column chromatography (SiO₂, Petroleum ether/Ethyl acetate=10/1 to 3/1). 6 (130 mg, 217.74 μmol, 50% yield) was obtained as a yellow solid.

¹H NMR (400 MHz, CHLOROFORM-d) δ=8.62 (d, J=2.0 Hz, 1H), 8.19 (d, J=8.0 Hz, 2H), 7.68 (dd, J=2.4, 8.4 Hz, 1H), 7.57 (d, J=8.4 Hz, 1H), 6.82-6.77 (m, 1H), 6.74-6.69 (m, 2H), 4.01 (s, 3H), 3.99-3.98 (m, 1H), 3.99 (s, 2H), 3.97-3.91 (m, 2H), 2.97 (s, 2H), 2.78 (s, 1H), 2.35 (s, 2H), 2.26 (t, J=18.8 Hz, 4H), 1.94-1.73 (m, 4H)

### General Procedure for Preparation of Compound 87 (7):

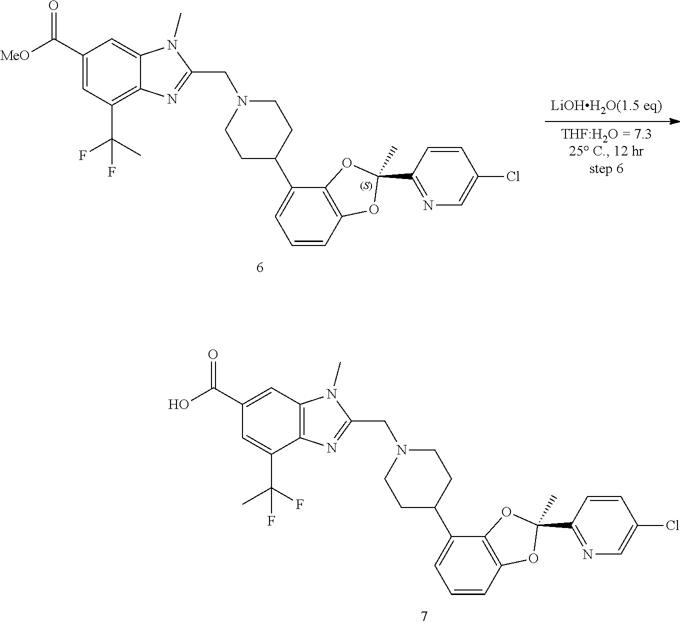

To a solution of 6 (120 mg, 200.99 μmol, 1 eq) in THF (0.7 mL) was added LiOH H₂O (12.65 mg, 301.48 μmol, 1.5 eq) in H₂O (0.3 mL). The mixture was stirred at 25° C. for 12 hr. LC-MS (Rt=1.495) showed 6 was consumed completely and desired mass was detected. The mixture was purified by prep-HPLC (FA condition, column: Phenomenex Luna C18 100*30 mm*3 um; mobile phase: [H₂O (0.2% FA)-ACN]; gradient: 15%-55% B over 8.0 min). (S)-2-((4-(2-(5-Chloropyridin-2-yl)-2-methylbenzo[d][1,3]dioxol-4-yl)piperidin-1-yl)methyl)-4-(1,1-difluoroethyl)-1-methyl-1H-benzo[d]imidazole-6-carboxylic acid (29.45 mg, 50.51 μmol, 25.13% yield) was obtained as a white solid.

LCMS: RT=2.438 min, MS cal.: 582.2, 584.2, [M+H]⁺=583.2, 585.2

HPLC: RT=11.663 min, purity: 97.27%

¹H NMR (400 MHz, METHANOL-d4) δ=8.59 (d, J=2.0 Hz, 1H), 8.30 (s, 1H), 8.11 (s, 1H), 7.86 (dd, J=2.4, 8.4 Hz, 1H), 7.64 (d, J=8.4 Hz, 1H), 6.81-6.68 (m, 3H), 4.06 (s, 2H), 4.02 (s, 3H), 3.16 (d, J=8.6 Hz, 2H), 2.83-2.74 (m, 1H), 2.47 (t, J=10.4 Hz, 2H), 2.18 (t, J=18.8 Hz, 3H), 2.01 (s, 3H), 1.98-1.81 (m, 4H)

### Example A280—Preparation of Compound 88. (S)-2-((4-(2-(5-Chloropyridin-2-yl)-2-methylbenzo[d][1,3]dioxol-4-yl)piperidin-1-yl)methyl)-1-ethyl-4-isopropoxy-1H-benzo[d]imidazole-6-carboxylic acid (Compound 88) (7)

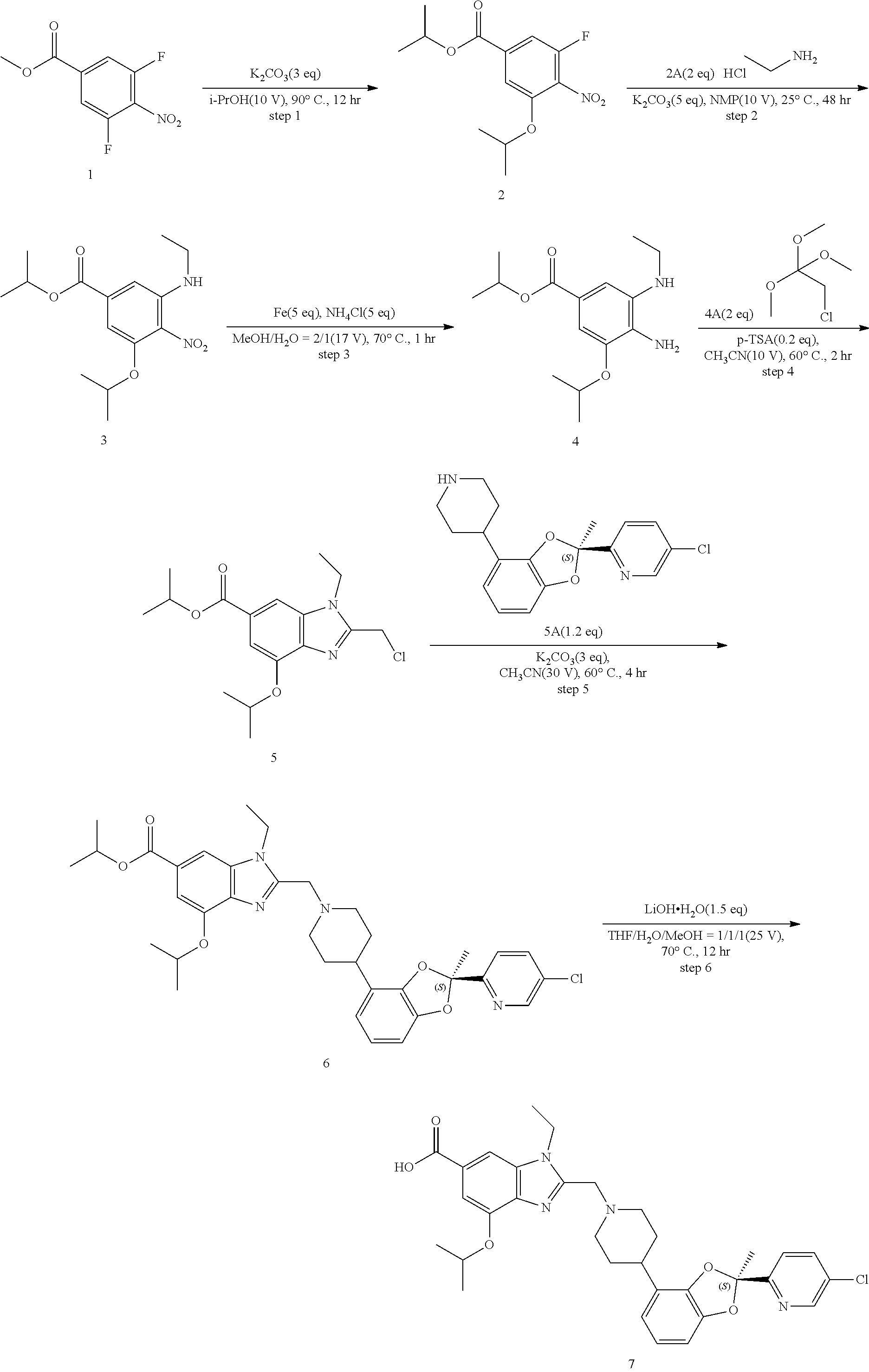

### General Procedure for Preparation of Intermediate 2:

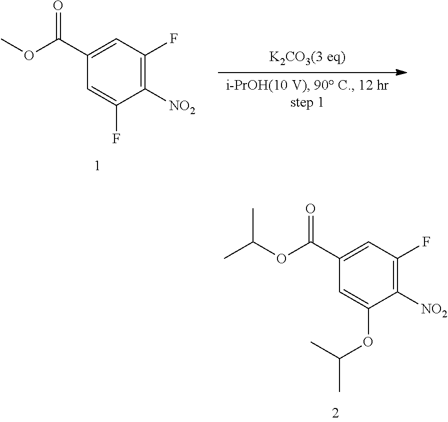 [SMILES: COC(=O)c1cc(F)c([N+](=O)[O-])c(F)c1 ; "K2CO3(3 eq)" ; "i-PrOH(10 V), 90° C., 12 hrstep 1" ; CC(C)OC(=O)c1cc(F)c([N+](=O)[O-])c(OC(C)C)c1]

A mixture of 1 (5 g, 23.03 mmol, 1 eq) and K₂CO₃ (9.55 g, 69.08 mmol, 3 eq) in i-PrOH (50 mL) was degassed and purged with N₂ for 3 times, then the mixture was stirred at 90° C. for 12 hr under N₂ atmosphere. LCMS (product: RT=2.190 min; by product: RT=1.937 min) showed the starting material was consumed completely. The reaction mixture was quenched by H₂O 30 mL at 20° C., then extracted with ethyl acetate (50 mL*3). The combined organic layers were washed with brine (20 mL*2), dried over Na₂SO₄, filtered, and concentrated under reduced pressure to give a residue. The residue was purified by column chromatography (SiO₂, Petroleum ether/Ethyl acetate=100/1 to 90/1). 2 (1.7 g, 5.96 mmol, 25.88% yield) was obtained as a white oil.

¹H NMR (400 MHz, CHLOROFORM-d) δ=7.51 (s, 1H), 7.44 (dd, J=1.2, 9.2 Hz, 1H), 5.32-5.20 (m, 1H), 4.76 (td, J=6.0, 12.0 Hz, 1H), 1.40 (s, 6H), 1.39 (s, 6H)

### General Procedure for Preparation of Intermediate 3:

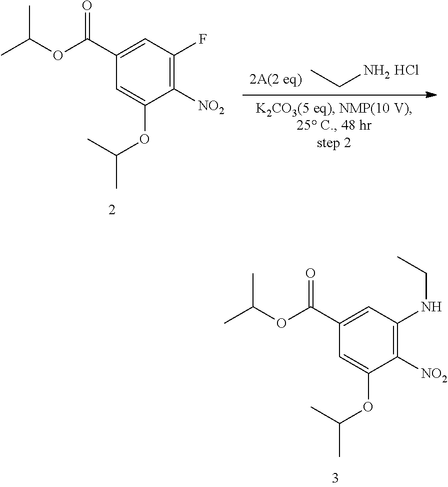

A mixture of 2 (800 mg, 2.80 mmol, 1 eq), 2A (661.86 mg, 5.61 mmol, 2 eq, HCl) and K₂CO₃ (1.94 g, 14.02 mmol, 5 eq) in NMP (8 mL) was degassed and purged with N₂ for 3 times, and then the mixture was stirred at 20° C. for 48 hr under N₂ atmosphere. HPLC showed the starting material was consumed completely. The reaction mixture was quenched by H₂O 5 mL at 20° C., then extracted with ethyl acetate (5 mL*3). The combined organic layers were washed with brine (5 mL*2), dried over Na₂SO₄, filtered, and concentrated under reduced pressure to give a residue. The residue was purified by column chromatography (SiO₂, Petroleum ether/Ethyl acetate=100/1 to 50/1). 3 (700 mg, 2.26 mmol, 80.43% yield) was obtained as a yellow oil.

¹H NMR (400 MHz, CHLOROFORM-d) δ=7.01 (d, J=1.6 Hz, 1H), 6.92 (d, J=1.6 Hz, 1H), 5.24 (td, J=6.4, 12.6 Hz, 1H), 4.75-4.64 (m, 1H), 3.27 (q, J=7.2 Hz, 2H), 1.38 (t, J=6.0 Hz, 12H), 1.30 (t, J=7.2 Hz, 3H)

### General Procedure for Preparation of Intermediate 4:

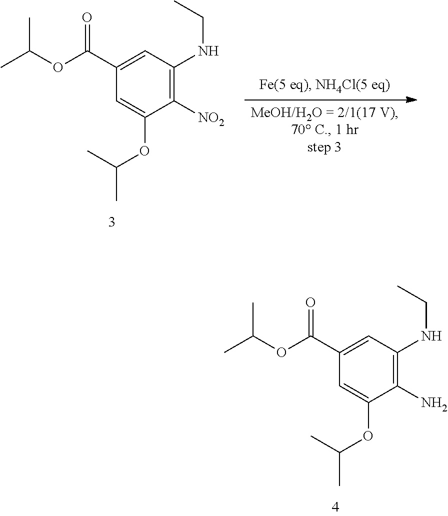

MeOH (4 mL) was charged to the three-necked round bottom flask, then 3 (350 mg, 1.13 mmol, 1 eq) and NH₄Cl (301.63 mg, 5.64 mmol, 5 eq) in H₂O (2 mL) was added to the mixture at 20° C. At 20° C., Fe (314.90 mg, 5.64 mmol, 5 eq) was added in portions to the reaction mixture at 20° C. After the addition, the mixture was stirred at 70° C. for 1 hr. LCMS (product: RT=0.407 min) showed the starting material was consumed completely. The reaction mixture was cooled down to 20° C. and filtered, the filtrate was added to H₂O 5 mL at 20° C. The mixture was extracted with ethyl acetate (5 mL*3). The combined organic phase was washed with brine (5 mL*2), dried with Na₂SO₄ then filtered. The organic phase was concentrated under reduced pressure at 35° C. to give a residue. The residue was purified by column chromatography (SiO₂, Petroleum ether/Ethyl acetate=100/1 to 50/1). 4 (280 mg, 998.71 μmol, 88.56% yield) was obtained as a white solid.

LCMS: RT=0.421 min, MS cal.: 280.2, [M+H]⁺=281.1

¹H NMR (400 MHz, CHLOROFORM-d) δ=7.18 (s, 1H), 7.11 (d, J=1.2 Hz, 1H), 5.27-5.16 (m, 1H), 4.67-4.55 (m, 1H), 4.02-3.32 (m, 2H), 3.22 (q, J=7.2 Hz, 2H), 1.37 (d, J=1.6 Hz, 6H), 1.35 (d, J=2.0 Hz, 7H), 1.32 (s, 2H)

### General Procedure for Preparation of Intermediate 5:

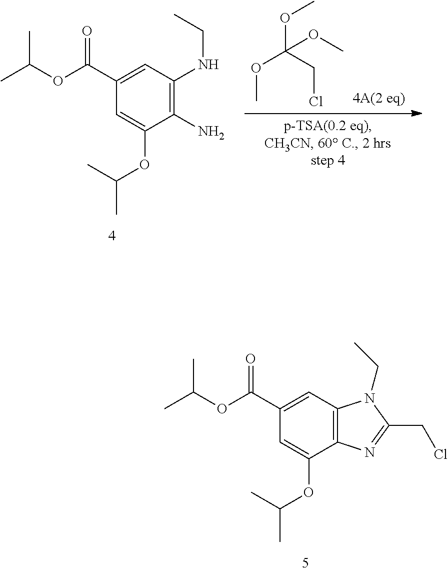

A mixture of 4 (220 mg, 784.70 μmol, 1 eq), 4A (242.62 mg, 1.57 mmol, 211.52 μL, 2 eq) and p-TSA (27.03 mg, 156.94 μmol, 0.2 eq) in CH₃CN (3 mL) was degassed and purged with N₂ for 3 times, and then the mixture was stirred at 60° C. for 2 hr under N₂ atmosphere. LCMS (product: RT=0.537 min) showed the starting material was consumed completely. The reaction mixture was concentrated under reduced pressure to remove solvent. 5 (300 mg, crude) was obtained as a white solid.

¹H NMR (400 MHz, CHLOROFORM-d) δ=7.65 (s, 1H), 7.37 (s, 1H), 5.22 (spt, J=6.4 Hz, 1H), 4.90-4.83 (m, 1H), 4.30 (q, J=7.2 Hz, 2H), 3.74 (s, 1H), 3.44-3.39 (m, 1H), 1.48 (t, J=7.2 Hz, 3H), 1.40 (d, J=6.4 Hz, 6H), 1.34 (d, J=6.4 Hz, 6H)

### General Procedure for Preparation of Intermediate 6:

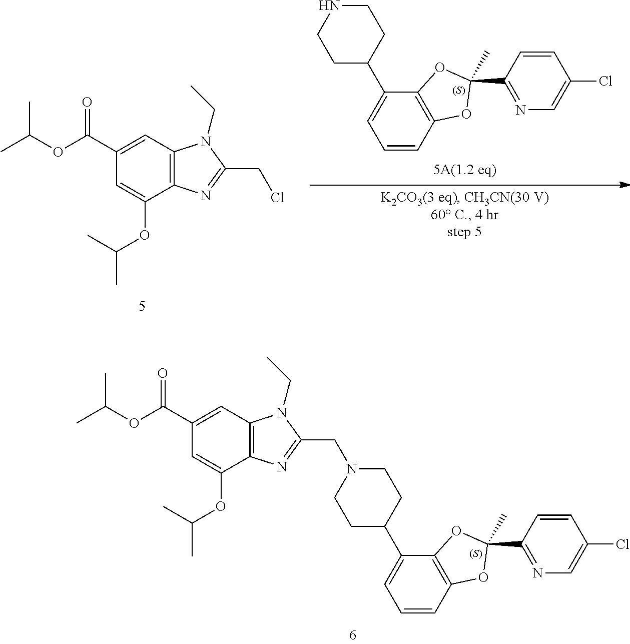

A mixture of 5 (100 mg, 295.13 μmol, 1 eq), 5A (117.16 mg, 354.16 μmol, 1.2 eq) and K₂CO₃ (203.95 mg, 1.48 mmol, 5 eq) in CH₃CN (3 mL) was degassed and purged with N₂ for 3 times, then the mixture was stirred at 60° C. for 2 hr under N₂ atmosphere. LCMS (ET64759-357-P1A, product: RT=0.548 min) showed the starting material was consumed completely. The reaction mixture was quenched by H₂O 5 mL at 20° C. and extracted with ethyl acetate (5 mL*3). The combined organic layers were washed with brine (5 mL*2), dried over Na₂SO₄, filtered, and concentrated under reduced pressure to give a residue. The residue was purified by column chromatography (SiO₂, Petroleum ether/Ethyl acetate=75/1 to 50/1). Compound 6 (120 mg, 189.52 μmol, 64.22% yield) was obtained as a white solid.

¹H NMR (400 MHz, CHLOROFORM-d) δ=8.62 (d, J=2.0 Hz, 1H), 7.72-7.65 (m, 2H), 7.57 (s, 1H), 7.40 (s, 1H), 6.82-6.75 (m, 1H), 6.73-6.67 (m, 2H), 5.34-5.25 (m, 1H), 4.98 (s, 1H), 4.42 (d, J=7.2 Hz, 2H), 3.89 (s, 2H), 2.96 (d, J=5.6 Hz, 2H), 2.83-2.67 (m, 1H), 2.39-2.22 (m, 2H), 2.05 (s, 3H), 1.94-1.70 (m, 5H), 1.50 (s, 3H), 1.49 (d, J=6.0 Hz, 6H), 1.41 (d, J=6.4 Hz, 6H)

### General Procedure for Preparation of Compound 88 (7):

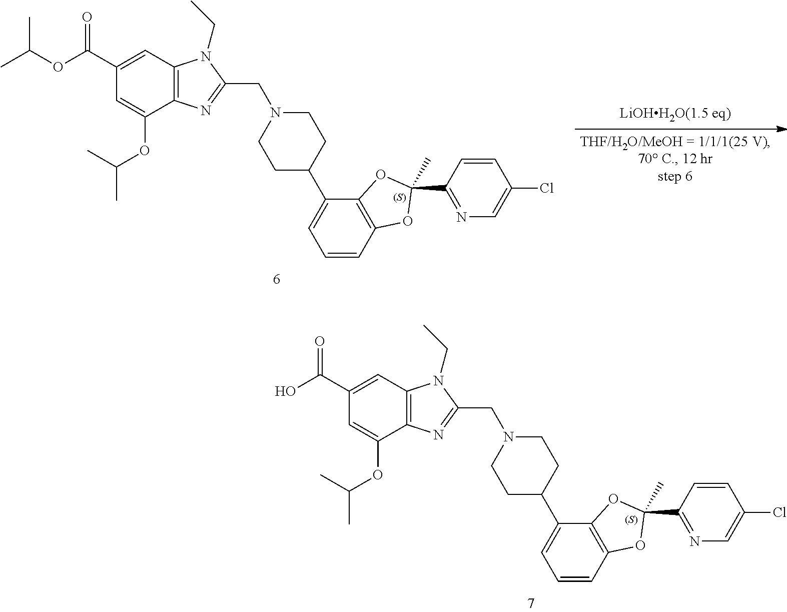

A mixture of 6 (120 mg, 189.52 μmol, 1 eq), LiOH·H₂O (11.93 mg, 284.28 μmol, 1.5 eq) in THF (1 mL), H₂O (1 mL) and MeOH (1 mL) was degassed and purged with N₂ for 3 times, and then the mixture was stirred at 70° C. for 12 hr under N₂ atmosphere. LCMS (product: RT=1.610 min) showed the starting material was consumed completely. The reaction mixture was concentrated under reduced pressure to remove solvent. The residue was purified by prep-HPLC (column: Waters Xbridge Prep OBD C18 150*40 mm*10 um; mobile phase: [H₂O (10 mM NH₄HCO₃)—CH₃CN]; gradient: 15%-65% B over 8.0 min). 7 (26.67 mg, 45.12 μmol, 23.81% yield, 100% purity) was obtained as a white solid.

LCMS: RT=2.718 min, MS cal.: 590.2, [M+H]⁺=591.3

HPLC: RT=11.698 min, purity: 100%

¹H NMR (400 MHz, DMSO-d6) δ=8.72 (dd, J=0.8, 2.4 Hz, 1H), 8.00 (dd, J=2.8, 8.4 Hz, 1H), 7.73 (d, J=1.2 Hz, 1H), 7.63-7.54 (m, 1H), 7.24 (d, J=1.2 Hz, 1H), 6.83-6.65 (m, 3H), 5.02 (td, J=6.4, 12.2 Hz, 1H), 4.37 (q, J=7.2 Hz, 2H), 3.79 (s, 2H), 2.92 (d, J=10.0 Hz, 2H), 2.72-2.58 (m, 1H), 2.19 (t, J=10.4 Hz, 2H), 1.99 (s, 3H), 1.81-1.61 (m, 4H), 1.39 (t, J=7.2 Hz, 3H), 1.33 (d, J=6.0 Hz, 6H)

### Example B1: GLP-1R Cell Assay

Stable cell lines expressing high and low GLP-1R surface expression were generated in CHO-K1 cells transfected (Fugene 6) with a puromycin selectable DNA plasmid encoding human GLP-1R receptor (accession number: NM_002062.5) under control of an EF1A promoter. Transfected cells were seeded into 24-well plates (9,000 cells/well) containing complete medium and incubated in a humidified incubator at 37° C. with 5% carbon dioxide. After overnight incubation, medium was replaced with complete medium supplemented with puromycin (6 μg/mL) and refreshed every 2-3 days to select for stably transfected cells. Individual pools of selected cells were expanded prior to analysis for responsiveness to GLP-1 control peptide using a TR-FRET assay to detect cAMP (LANCE Ultra CAMP Assay, Perkin Elmer). Briefly, cells were collected in Versene solution, plated in 384-well plates (1,000 cells/well) and combined with serially diluted GLP-IR control peptide (10 nL) using an acoustic dispenser (ECHO). Plates were incubated for 30 minutes at 25° C. prior to the addition of EU-CAMP tracer (5 μL) and Ulight-anti-CAMP (5 μL) reagents to each well, followed by 15 minutes incubation at 25° C. TR-FRET signal was detected using an En Vision Multimode Plate Reader (excitation=320 nm; emission=615 and 655 nm). Dose-response curves were used to generate EC₅₀ values as a measure of responsiveness to the GLP-IR control peptide. Selected cell lines were monitored for responsiveness over multiple passages to ensure stability. CHO-K1_hGLP-1Rhigh_clone16 and CHO-K1 hGLP-1Rlow_clone10 showed consistently high and low responsiveness to GLP-IR control peptide, respectively, and were chosen for further analysis to determine relative levels of GLP-IR surface expression. Briefly, GLP-IR expression was analyzed by flow cytometry using a fluorescein-labeled Exendin-4 peptide fluorescent probe (FLEX). Cells were harvested in Versene solution and washed 3-times with PBS+0.5% BSA before incubation with FLEX reagent (10 μM) for 2 hours at room temperature. After incubation, cells were washed 3-times in PBS+0.5% BSA before final resuspension in PBS prior to analysis by flow cytometry to measure FLEX mean fluorescence intensity (MFI) as a measure of GLP-IR expression on the cell surface. Both cell lines showed higher MFI values relative to control CHO-K1 cells, confirming GLP-IR surface expression; CHO-K1_hGLP-1Rhigh_clone16 cells showed significantly higher MFI levels relative to CHO-K1-hGLP-1low_clone10 cells.

For compound testing in the CHO-K1_hGLP-IRlow_clone10 cell line, cells were seeded in 384-well plates (1,000 cells/well). Test compounds were serially diluted in DMSO (10-point, 3-fold dilution), added to wells using an ECHO dispenser (10 nL/well) and plates were centrifuged for 1 min and agitated for 2 min at room temperature prior to 30-minute incubation at 25° C. After incubation, Eu-CAMP (5 μL) and Ulight-anti-cAMP (5 μL) reagents were added to each well, followed by centrifugation for 1 minute, agitation for 2 minutes at room temperature, and final incubation of the plates at 25° C. for 15 minutes. Plates were read using an EnVision microplate reader (excitation=320 nm; emission=615 and 655 nm). Dose-response curves were generated from duplicate wells based on percent activation calculated relative to a control GLP-1 peptide agonist that was run in parallel. EC₅₀ values were determined by fitting percent activation as a function of compound concentration using the Hill equation (XLfit). The EC₅₀ values of exemplary compounds in the low expression assay are shown in Table 2 below, wherein “+” indicates that the EC₅₀ is >1,000 nM; “++” indicates EC₅₀ is <1,000 nM and >100 nM; “+++” indicates that the EC₅₀ is <100 nM and >10 nM; and “++++” indicates that the EC₅₀ is <10 nM. The maximum activation values (Emax) of exemplary compounds are shown in Table 2a below, wherein “+” indicates that the Emax is <25%; “++” indicates Emax is <50% and >25%; “+++” indicates that the Emax is <85% and >50%; and “++++” indicates that the Emax is >85%.

The compounds tested were compound samples prepared according to the General Procedures described in the Examples section.

TABLE 2

| Cmpd | GLP-1R Low Expression Cell |
|---|---|
| No. | Assay EC50 (nM) |
| 1 | ++++ |
| 2 | ++++ |
| 3 | ++ |
| 4 | ++++ |
| 5 | + |
| 6 | ++++ |
| 7 | +++ |
| 8 | ++++ |
| 9 | ++++ |
| 30 | ++++ |
| 32 | ++++ |
| 57 | ++++ |
| 63 | ++++ |
| 71 | +++ |
| 72 | ++++ |
| 73 | ++++ |
| 74 | +++ |
| 75 | + |
| 76 | + |
| 77 | ++++ |
| 78 | ++++ |
| 79 | ++ |
| 80 | ++++ |
| 81 | ++++ |
| 82 | ++++ |
| 83 | ++++ |
| 84 | ++++ |
| 85 | ++++ |
| 86 | ++ |
| 87 | ++ |
| 88 | +++ |
| 89 | ++++ |
| 90 | ++++ |
| 91 | ++ |
| 92 | ++++ |
| 93 | ++++ |
| 94 | + |
| 95 | ++++ |
| 96 | +++ |
| 97 | + |
| 98 | ++++ |
| 99 | + |
| 100 | ++++ |
| 101 | +++ |
| 102 | +++ |
| 103 | + |
| 104 | ++ |
| 105 | ++++ |
| 106 | ++++ |
| 107 | + |
| 108 | ++++ |
| 109 | ++++ |
| 110 | ++++ |
| 111 | ++ |
| 112 | ++ |
| 113 | ++++ |
| 114 | ++++ |
| 115 | ++++ |
| 116 | +++ |
| 117 | ++++ |
| 118 | ++++ |
| 119 | +++ |
| 120 | ++++ |
| 121 | ++++ |
| 122 | + |
| 123 | +++ |
| 124 | + |
| 125 | +++ |
| 126 | + |
| 127 | ++++ |
| 128 | +++ |
| 129 | +++ |
| 130 | + |
| 131 | ++ |
| 132 | ++++ |
| 133 | ++++ |
| 134 | +++ |
| 135 | ++ |
| 136 | ++++ |
| 137 | +++ |
| 138 | + |
| 139 | +++ |
| 140 | +++ |
| 141 | +++ |
| 143 | ++++ |
| 144 | + |
| 145 | ++ |
| 146 | + |
| 147 | ++ |
| 148 | ++ |
| 149 | +++ |
| 150 | +++ |
| 151 | ++ |
| 152 | +++ |
| 153 | +++ |
| 154 | ++++ |
| 155 | +++ |
| 156 | ++++ |
| 157 | ++ |
| 158 | + |
| 159 | ++ |
| 160 | ++ |
| 142 | + |
| 167 | + |
| 168 | +++ |
| 169 | ++++ |
| 170 | ++++ |
| 171 | + |
| 173 | + |
| 174 | + |
| 175 | ++++ |
| 176 | ++ |
| 177 | + |
| 178 | ++++ |
| 179 | + |
| 180 | +++ |
| 181 | + |
| 182 | ++ |
| 183 | ++++ |
| 184 | ++ |
| 185 | ++ |
| 186 | ++ |
| 187 | + |
| 188 | +++ |
| 189 | +++ |
| 190 | +++ |
| 191 | ++ |
| 192 | + |
| 193 | + |
| 194 | ++ |
| 195 | +++ |
| 196 | +++ |
| 197 | ++++ |
| 198 | +++ |
| 199 | +++ |
| 200 | + |
| 201 | +++ |
| 202 | +++ |
| 203 | +++ |
| 204 | + |
| 205 | + |
| 206 | + |
| 207 | ++++ |
| 208 | +++ |
| 209 | +++ |
| 210 | + |
| 211 | +++ |
| 212 | ++ |
| 213 | ++ |
| 214 | ++ |
| 215 | ++++ |
| 216 | +++ |
| 217 | + |
| 218 | +++ |
| 219 | + |
| 220 | + |
| 221 | +++ |
| 222 | ++ |
| 223 | +++ |
| 224 | +++ |
| 225 | ++++ |
| 226 | ++++ |
| 227 | ++++ |
| 228 | +++ |
| 229 | ++ |
| 230 | + |
| 231 | +++ |
| 232 | + |
| 233 | ++ |
| 234 | + |
| 235 | +++ |
| 236 | ++ |
| 237 | ++++ |
| 238 | +++ |
| 239 | + |
| 240 | + |
| 241 | + |
| 242 | +++ |
| 243 | ++ |
| 244 | + |
| 245 | ++++ |
| 246 | ++++ |
| 247 | ++ |
| 248 | ++ |
| 249 | ++++ |
| 250 | +++ |
| 251 | ++ |
| 252 | +++ |
| 253 | +++ |
| 254 | ++ |
| 255 | ++ |
| 256 | ++++ |
| 257 | +++ |
| 258 | ++ |
| 259 | ++ |
| 260 | +++ |
| 261 | +++ |
| 262 | +++ |
| 263 | ++++ |
| 264 | + |
| 265 | +++ |
| 266 | ++++ |
| 267 | ++ |
| 268 | ++++ |
| 269 | ++ |
| 270 | +++ |
| 271 | ++ |
| 272 | ++++ |
| 273 | ++++ |
| 274 | + |
| 275 | +++ |
| 276 | +++ |
| 277 | + |
| 278 | ++ |
| 279 | ++++ |
| 280 | + |
| 281 | + |
| 282 | ++++ |
| 283 | ++++ |
| 284 | +++ |
| 285 | ++++ |
| 286 | ++++ |
| 287 | + |
| 288 | +++ |
| 289 | ++ |
| 290 | ++++ |
| 291 | ++++ |
| 292 | ++++ |
| 293 | ++++ |
| 294 | + |
| 295 | ++++ |
| 296 | ++++ |
| 297 | ++++ |
| 298 | ++++ |
| 299 | ++++ |
| 300 | ++ |
| 301 | +++ |
| 302 | ++++ |
| 303 | +++ |
| 304 | ++++ |
| 305 | +++ |
| 306 | ++++ |
| 307 | +++ |
| 308 | ++++ |
| 309 | ++++ |
| 310 | +++ |
| 311 | +++ |
| 312 | +++ |
| 313 | +++ |
| 314 | + |
| 315 | +++ |
| 316 | ++ |
| 317 | ++ |
| 318 | ++ |
| 319 | +++ |
| 320 | +++ |
| 321 | +++ |
| 322 | +++ |
| 323 | +++ |
| 324 | +++ |
| 325 | ++ |
| 326 | + |
| 327 | ++++ |
| 328 | +++ |
| 329 | ++++ |
| 330 | +++ |
| 331 | +++ |
| 332 | ++++ |
| 333 | ++ |
| 334 | +++ |
| 335 | + |
| 336 | + |
| 337 | +++ |
| 338 | + |
| 339 | ++ |
| 340 | +++ |
| 341 | ++ |
| 342 | ++++ |
| 343 | ++++ |
| 344 | ++++ |
| 345 | + |
| 346 | +++ |
| 347 | + |
| 348 | ++++ |
| 349 | ++++ |
| 350 | ++ |
| 351 | +++ |
| 352 | ++ |
| 353 | +++ |
| 354 | +++ |
| 355 | ++ |
| 356 | +++ |
| 357 | ++++ |
| 538 | ++++ |
| 359 | ++++ |
| 360 | ++++ |
| 361 | +++ |
| 362 | + |
| 363 | ++++ |
| 364 | +++ |
| 365 | +++ |
| 366 | ++++ |
| 367 | + |
| 368 | + |
| 369 | + |
| 370 | ++ |
| 371 | + |
| 372 | ++++ |
| 373 | + |
| 374 | ++ |
| 375 | ++ |
| 376 | ++++ |
| 377 | ++++ |
| 378 | +++ |
| 379 | +++ |
| 380 | +++ |
| 381 | ++++ |
| 382 | ++++ |
| 383 | +++ |
| 384 | +++ |
| 386 | +++ |
| 387 | ++++ |

TABLE 2a

| Cmpd | GLP-1R Low Expression Cell |
|---|---|
| No. | Assay Emax (%) |
| 1 | ++++ |
| 2 | ++++ |
| 3 | ++++ |
| 4 | ++++ |
| 5 | + |
| 6 | ++++ |
| 7 | ++++ |
| 8 | ++++ |
| 9 | ++++ |
| 71 | ++++ |
| 72 | ++++ |
| 73 | ++++ |
| 74 | ++++ |
| 75 | + |
| 76 | +++ |
| 77 | ++++ |
| 78 | ++++ |
| 79 | ++++ |
| 80 | ++++ |
| 81 | ++++ |
| 82 | ++++ |
| 83 | ++++ |
| 84 | ++++ |
| 85 | ++++ |
| 86 | ++++ |
| 87 | ++++ |
| 88 | ++++ |
| 89 | ++++ |
| 90 | ++++ |
| 91 | ++++ |
| 92 | ++++ |
| 93 | ++++ |
| 94 | ++++ |
| 95 | ++++ |
| 96 | ++++ |
| 97 | ++++ |
| 98 | ++++ |
| 99 | ++++ |
| 100 | ++++ |
| 101 | ++++ |
| 102 | ++++ |
| 103 | +++ |
| 104 | ++++ |
| 105 | ++++ |
| 106 | ++++ |
| 107 | +++ |
| 108 | ++++ |
| 109 | ++++ |
| 110 | ++++ |
| 111 | ++++ |
| 112 | ++++ |
| 113 | ++++ |
| 114 | ++++ |
| 115 | ++++ |
| 116 | ++++ |
| 117 | ++++ |
| 118 | ++++ |
| 119 | +++ |
| 120 | ++++ |
| 121 | ++++ |
| 122 | ++++ |
| 123 | ++++ |
| 124 | ++ |
| 125 | ++++ |
| 126 | +++ |
| 127 | ++++ |
| 128 | ++++ |
| 129 | ++++ |
| 130 | ++ |
| 131 | ++++ |
| 132 | ++++ |
| 133 | ++++ |
| 134 | ++++ |
| 135 | ++++ |
| 136 | ++++ |
| 137 | ++++ |
| 138 | ++ |
| 139 | ++++ |
| 140 | ++++ |
| 141 | ++++ |
| 142 | ++++ |
| 143 | ++++ |
| 144 | + |
| 145 | ++++ |
| 146 | +++ |
| 147 | ++++ |
| 148 | ++++ |
| 149 | ++++ |
| 150 | ++++ |
| 151 | ++++ |
| 152 | ++++ |
| 153 | ++++ |
| 154 | ++++ |
| 155 | ++++ |
| 156 | ++++ |
| 157 | ++++ |
| 158 | +++ |
| 159 | ++++ |
| 160 | ++++ |
| 167 | ++ |
| 168 | ++++ |
| 169 | ++++ |
| 170 | ++++ |
| 171 | + |
| 172 | + |
| 173 | + |
| 174 | + |
| 175 | ++++ |
| 177 | +++ |
| 178 | ++++ |
| 179 | ++ |
| 180 | ++++ |
| 181 | +++ |
| 182 | ++++ |
| 183 | ++++ |
| 184 | ++++ |
| 185 | ++++ |
| 186 | ++++ |
| 187 | + |
| 188 | ++++ |
| 189 | ++++ |
| 190 | ++++ |
| 191 | ++++ |
| 192 | +++ |
| 193 | + |
| 194 | ++++ |
| 195 | ++++ |
| 196 | ++++ |
| 197 | ++++ |
| 198 | ++++ |
| 199 | ++++ |
| 200 | ++++ |
| 201 | ++++ |
| 202 | ++++ |
| 203 | ++++ |
| 204 | ++++ |
| 205 | +++ |
| 206 | + |
| 207 | ++++ |
| 208 | ++++ |
| 209 | ++++ |
| 210 | ++ |
| 211 | ++++ |
| 212 | ++++ |
| 213 | ++++ |
| 214 | +++ |
| 215 | ++++ |
| 216 | ++++ |
| 217 | ++++ |
| 218 | ++++ |
| 219 | ++ |
| 220 | ++++ |
| 221 | ++++ |
| 222 | ++++ |
| 223 | ++++ |
| 224 | ++++ |
| 225 | ++++ |
| 226 | ++++ |
| 227 | ++++ |
| 228 | ++++ |
| 229 | ++++ |
| 230 | + |
| 231 | ++++ |
| 232 | ++ |
| 233 | ++++ |
| 234 | + |
| 235 | ++++ |
| 236 | +++ |
| 237 | ++++ |
| 238 | ++++ |
| 239 | +++ |
| 240 | + |
| 241 | + |
| 242 | ++++ |
| 243 | +++ |
| 244 | + |
| 245 | ++++ |
| 246 | ++++ |
| 247 | ++++ |
| 248 | ++++ |
| 249 | ++++ |
| 255 | +++ |
| 256 | ++++ |
| 257 | +++ |
| 258 | ++++ |
| 259 | ++++ |
| 260 | ++++ |
| 261 | ++++ |
| 262 | ++++ |
| 263 | ++++ |
| 264 | ++ |
| 265 | ++++ |
| 266 | ++++ |
| 267 | ++++ |
| 268 | ++++ |
| 269 | ++++ |
| 270 | ++++ |
| 271 | +++ |
| 272 | ++++ |
| 273 | ++++ |
| 274 | + |
| 275 | ++++ |
| 276 | ++++ |
| 277 | +++ |
| 278 | ++++ |
| 279 | +++ |
| 280 | +++ |
| 281 | +++ |
| 282 | ++++ |
| 283 | ++++ |
| 284 | ++++ |
| 285 | ++++ |
| 286 | ++++ |
| 287 | ++++ |
| 288 | ++++ |
| 289 | ++++ |
| 290 | ++++ |
| 291 | ++++ |
| 292 | ++++ |
| 293 | ++++ |
| 294 | +++ |
| 295 | ++++ |
| 296 | ++++ |
| 297 | ++++ |
| 298 | ++++ |
| 299 | ++++ |
| 300 | ++++ |
| 301 | ++++ |
| 302 | ++++ |
| 303 | ++++ |
| 304 | ++++ |
| 305 | ++++ |
| 306 | ++++ |
| 307 | ++++ |
| 308 | ++++ |
| 309 | ++++ |
| 310 | ++++ |
| 311 | ++++ |
| 312 | ++++ |
| 313 | ++++ |
| 314 | ++ |
| 315 | ++++ |
| 316 | +++ |
| 317 | ++++ |
| 318 | ++++ |
| 319 | ++++ |
| 320 | ++++ |
| 321 | ++++ |
| 322 | ++++ |
| 323 | ++++ |
| 324 | ++++ |
| 325 | ++++ |
| 326 | + |
| 327 | ++++ |
| 328 | ++++ |
| 329 | ++++ |
| 330 | ++++ |
| 331 | ++++ |
| 332 | ++++ |
| 333 | ++++ |
| 334 | ++++ |
| 335 | +++ |
| 336 | + |
| 337 | ++++ |
| 338 | +++ |
| 339 | ++++ |
| 340 | ++++ |
| 341 | ++++ |
| 342 | ++++ |
| 343 | ++++ |
| 344 | ++++ |
| 345 | +++ |
| 346 | ++++ |
| 347 | ++ |
| 348 | ++++ |
| 349 | ++++ |
| 350 | ++++ |
| 351 | ++++ |
| 352 | ++++ |
| 353 | ++++ |
| 354 | ++++ |
| 355 | ++++ |
| 356 | ++++ |
| 357 | ++++ |
| 358 | ++++ |
| 359 | ++++ |
| 360 | ++++ |
| 361 | ++++ |
| 362 | +++ |
| 363 | ++++ |
| 364 | ++++ |
| 365 | ++++ |
| 366 | ++++ |
| 367 | ++++ |
| 368 | ++ |
| 369 | + |
| 370 | ++++ |
| 371 | ++++ |
| 372 | ++++ |
| 373 | + |
| 374 | ++++ |
| 375 | ++++ |
| 376 | ++++ |
| 377 | ++++ |
| 378 | ++++ |
| 379 | ++++ |
| 380 | ++++ |
| 381 | ++++ |
| 382 | +++ |
| 383 | ++++ |
| 384 | ++++ |
| 386 | ++++ |

### Example B2: Metabolic Stability in Hepatocytes

Test compounds were incubated in rat and human hepatocytes and stability was assessed from the substrate depilation approach. Test compounds were dissolved in dimethyl sulfoxide (DMSO) to create a 10 mM Stock, and then further diluted to create a 1000× Working Stock of 1 mM with DMSO in 96-well plates for test compounds and the positive control (midazolam). Vials containing cryopreserved hepatocytes were removed from the liquid nitrogen tank and immediately immersed in a 37° C. water bath. The vials were shaken gently until the contents had thawed and were then immediately emptied into 48 mL of pre-warmed HT Medium in a 50 mL conical tube. Cells remaining in the vial were resuspended with 1.0 mL of pre-warmed HT Medium and added to the conical tube. The tube was capped and then gently inverted several times to resuspend the hepatocytes. The cell suspension was centrifuged at 50×g at room temperature for 5 minutes and the supernatant discarded. The cell pellet was loosened by gently swirling the centrifuge tube and was re-suspended in 4 mL of warm Dulbecco's Modified Eagle medium (DMEM). Cell density was determined by a cell counter by Nexcelom, and DMEM medium was added to obtain a target density of 1×106 cells/mL. The assay was carried out in 96-well microtiter plates. Test Compounds were incubated at 1 μM with 1×10⁶ cells/mL hepatocytes in DMEM for 0, 30, 60, 120 and 240 minutes. The incubation was carried out with gentle shaking at 37° C. under a humid atmosphere of 95% air/5% CO₂. The volume of the incubation mixture was 37 μL with a final 0.1% DMSO. At each of the time points, the incubation was stopped by adding 150 μL quenching solution (100% acetonitrile, 0.1% formic acid containing bucetin as an internal standard for positive ESI mode). Subsequently, the mixtures were vortexed for 20 min and centrifuged at 4,000 RPM at 10° C. The supernatant (80 μL) was transferred to a clean 96-well plate and analyzed by LC-MS/MS. Midazolam at 1 μM with a final 0.1% DMSO was included as a positive control to verify assay performance. The percent parent remaining, intrinsic and predicted hepatic clearance and t_{1/2} were calculated. All samples were analyzed by LC-MS/MS using an AB Sciex API 4000 instrument, coupled to a Shimadzu LC-20AD LC Pump system. Separation was achieved using a Waters Atlantis T3 dC18 reverse phase HPLC column (20 mm×2.1 mm) at a flow rate of 0.5 mL/min. The mobile phase consisted of 0.1% formic acid in water (solvent A) and 0.1% formic acid in 100% acetonitrile (solvent B). Elution conditions are detailed below.

| Time | Flow | % | % |
|---|---|---|---|
| (min) | (μL/min) | A | B |
| 0 | 500 | 98 | 2 |
| 0.30 | 500 | 98 | 2 |
| 1.40 | 500 | 2 | 98 |
| 2.20 | 500 | 2 | 98 |
| 2.21 | 500 | 98 | 2 |
| 3.00 | 500 | 98 | 2 |

The ion optics of each test compound were optimized for their declustering potential (DP), collection energy (CE), collision-cell exit potential (CXP) and used in a selected ion monitoring experiment in the positive ion mode. The peak area ratio of each test compound to internal standard was then evaluated for stability. The extent of metabolism was calculated based on the disappearance of the test compound, compared to its initial concentration. The initial rates of clearance of the test compound were calculated using the linear regression plot of semi-log % remaining of the compound versus time. The elimination rate constant (k) of the linear regression plot was then used to determine t_{1/2} and the intrinsic clearance (CLᵢₙₜ) using the following formula, where C_{hepatocyte} (million cells/mL) is the cell density of the incubation:

$$k = - slope$$

$${t}_{1 / 2} = 0.6 \; 93 / k$$

$${CL}_{int} = k / {C}_{hepatocyte}$$

This method of intrinsic clearance determination assumes that the test compound concentration is far below the Michaelis-Menten constant of the compound to its metabolizing enzymes.

The predicted hepatic clearance (CLₕₑₚ) was calculated using the well stirred method with the following formula with CL_{int (in vivo)} normalized based on liver weight:

$${CL}_{int ( in vivo )} = {CL}_{int} \times Hepatocellularity \times liver weight$$

$${CL}_{hep predicted} = ( {CL}_{int ( in vivo )} \times {Q}_{liver} ) / ( {CL}_{int ( in vivo )} + {Q}_{liver} )$$

$$Where {Q}_{liver} ( ( ml / min / kg ) is Liver Blood Flow$$

The relevant physiological parameters of liver weight, blood flow, and hepatocellularity for various species are listed below:

| | Liver Weight | Hepatocellularity | Liver Blood |
|---|---|---|---|
| | (g liver/kg | (106 cells/g | Flow (Qₗᵢᵥₑᵣ, |
| Species | body weight) | liver) | mL/min/kg |
| Human | 25.7 | 135 | 20.7 |
| Rat | 40 | 120 | 55.2 |

Results are presented in the Table below for the intrinsic clearance (Clint) (mL/min/kg) and half-life (t½).

| | Rat | | Human | |
|---|---|---|---|---|
| Cmpd | Clint | t1/2 | Clint | t1/2 |
| # | (mL/min/kg) | (min) | (mL/min/kg) | (min) |
| 1 | 68.74 ± 3.50 | 48.39 ± 2.46 | 14.18 ± 0.97 | 169.56 ± 11.56 |
| 8 | 27.39 ± 1.77 | 121.43 ± 7.83 | 6.18 ± 0.70 | 4.46 ± 0.67 |

Results are presented in the Table below for the predicted hepatic clearance.

| | Rat | Human |
|---|---|---|
| | Clhep predicted | Clhep predicted |
| Cmpd # | (mL/min/kg) | (mL/min/kg) |
| 1 | 30.61 ± 3.29 | 8.42 ± 0.92 |
| 8 | 18.31 ± 1.71 | 4.76 ± 0.68 |

### Example B3: Rat Pharmacokinetics

Intravenous dosing: Compounds were formulated at 1.0 mg/mL in a solution comprising 5% polyethylene glycol 400 and 95% (12% (w/v) sulfobutyl-β-cyclodextrin in water) (v/v). Formulated compounds were sterile filtered through a 0.22 micron filter before dosing. Compounds were administered to male, 7-11-week-old Sprague-Dawley rats by jugular vein cannula infusion over 30 minutes at a dose of 1 mg/kg. Oral dosing: Compounds were formulated at 1.0 mg/mL in a solution comprising 5% polyethylene glycol 400 and 95% (12% (w/v) sulfobutyl-β-cyclodextrin in water) (v/v). Formulated compounds were administered to male, 7-11 week old Sprague-Dawley rats by oral gavage at a dose of 10 mg/kg or 3 mg/kg.

Sample collection: Blood collections of about 0.2 mL per time point were performed from jugular vein or other suitable site of each animal, into pre-chilled commercial EDTA-K2 tubes and placed on wet ice until centrifugation. Blood samples were processed for plasma by centrifugation at approximately 4° C., 3,200 g for 10 min. Plasma was collected and transferred into pre-labeled 96 well plate or polypropylene tubes, quick frozen over dry ice and kept at −60° C. or lower until

LC-MS/MS analysis.

Data analysis: Plasma concentration versus time data was plotted in graph and analyzed by non-compartmental approaches using the Phoenix WinNonlin 6.3 software program. Related PK parameters were calculated according to dosing route, e.g., CL, V_{dss} and C₀ for intravenous administration, Cₘₐₓ, Tₘₐₓ or % F for extravascular administration, and T_{1/2}, AUC₍₀₋ₜ₎, AUC_{(0-inf)}, MRT₍₀₋ₜ₎, MRT_{(0-inf)} for all routes.

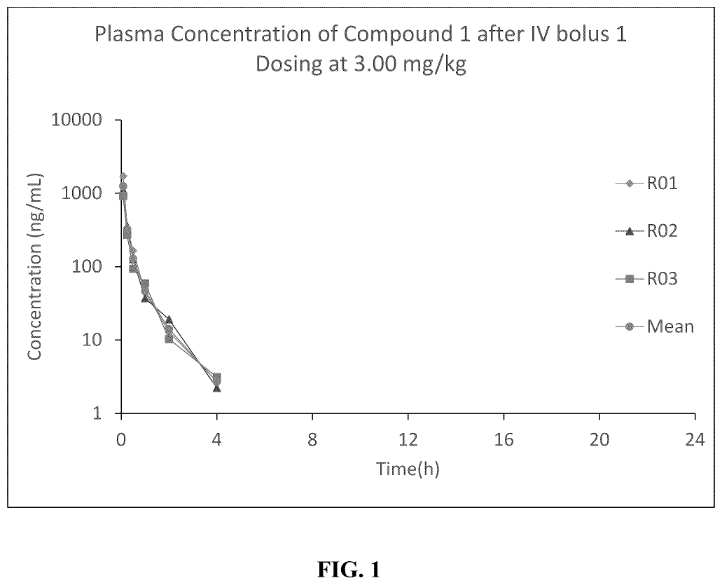

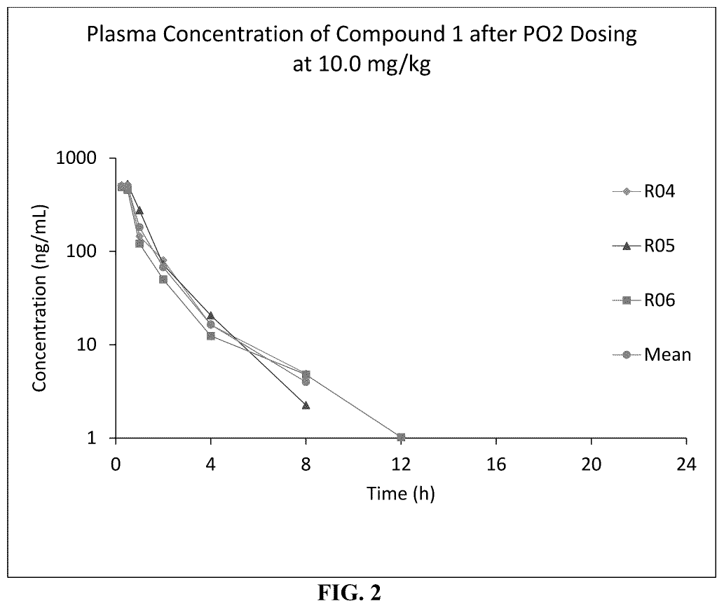

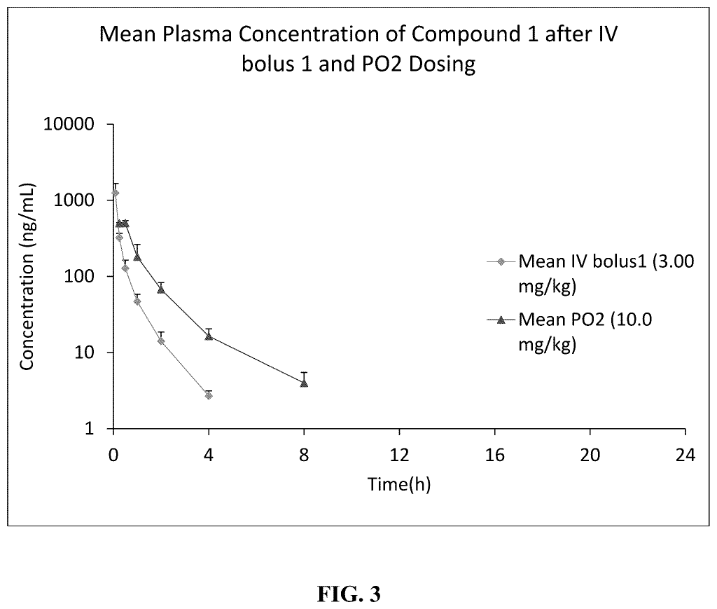

Results: PK parameters in plasma after intravenous dosing are shown in Table 3. PK parameters in plasma after oral dosing are shown in Tables 4 and 5. Plasma concentrations of Compound 1 after IV bolus dosing with 3 mg/kg are shown in FIG. 1. Plasma concentrations of Compound 1 oral dosing with 10 mg/kg are shown in FIG. 2. Mean plasma concentrations for compound 1 after IV bolus dosing at 3.0 mg/kg and IV bolus dosing at 10 mg/kg are shown in FIG. 3.

The C₀ values of exemplary compounds are shown in Tables 2, wherein “+” indicates that the C₀ is <500 ng/mL, “++” indicates that the C₀ is <1000 ng/ml and >500 ng/ml, “+++” indicates that the C₀ is <2000 ng/ml and >1000 ng/ml, and “++++” indicates that the C₀ is >2,000 ng/mL.

The Cₘₐₓ values of exemplary compounds are shown in Tables 3 and 4 below, wherein “+” indicates that the Cₘₐₓ is <500 ng/ml, “++” indicates that the Cₘₐₓ is <1000 ng/mL and >500 ng/mL, “+++” indicates that the Cₘₐₓ is <2000 ng/mL and >1000 ng/ml, “++++” indicates that the Cₘₐₓ is <5,000 ng/ml and >2,000 ng/ml, and “+++++” indicates that Cₘₐₓ is >5,000 ng/mL.

The T_{1/2} values of exemplary compounds are shown in Tables 3 and 4 below, wherein “+” indicates that the T_{1/2} is <1.5 h, “++” indicates that the T_{1/2} is <2.5h and >1.5h, and “+++” indicates that the T_{1/2} is ≥2.5 h.

The AUC values of exemplary compounds are shown in Tables 3 and 4 below, wherein “+” indicates that the AUC is <500 ng·h/mL, “++” indicates that the AUC is <2,500 ng·h/mL and >500 ng·h/mL, and “+++” indicates that the AUC is <10,000 ng·h/mL and >2,500 ng·h/mL, and “+++” indicates that the AUC is >10,000 ng·h/ml.

The bioavailability values of exemplary compounds are shown in Tables 4 and 5 below, wherein “+” indicates that the bioavailability is <25%; “++” indicates bioavailability is <50% and >25%; “+++” indicates that the bioavailability is <85% and >50%; and “++++” indicates that the bioavailability is >85%.

TABLE 3
IV Bolus Dosing of Compound 1 at 3.0 mg/kg

| Compound | Rat C₀ | Rat T_{1/2} | Rat AUC |
|---|---|---|---|
| No. | (ng/mL) | (h) | (ng · h/mL) |
| 1 | ++++ | + | + |

TABLE 4
Oral Dosing of Compound 1 at 10 mg/kg

| Compound | Rat Cₘₐₓ | Rat T_{1/2} | Rat AUC | Bioavailability |
|---|---|---|---|---|
| No. | (ng/mL) | (h) | (ng · h/mL) | (%) |
| 1 | ++ | ++ | ++ | ++ |

TABLE 5
Oral Dosing of Selected Compounds at 3 mg/kg

| Compound | Rat Cₘₐₓ | Rat T_{1/2} | Rat AUC | Bioavailability |
|---|---|---|---|---|
| No. | (ng/mL) | (h) | (ng · h/mL) | (%) |
| 6 | + | ++ | + | ++ |
| 7 | + | +++ | + | + |
| 8 | + | +++ | + | + |
| 71 | ++ | + | ++ | ++ |
| 72 | + | + | + | + |
| 73 | + | + | + | + |
| 77 | + | − | + | + |
| 78 | + | ++ | + | ++ |
| 80 | + | +++ | + | + |
| 81 | + | +++ | + | + |
| 83 | ++++ | +++ | +++ | ++++ |
| 85 | +++ | ++ | +++ | ++ |
| 89 | + | ++ | + | ++ |
| 90 | + | ++ | + | + |
| 111 | +++++ | ++ | ++++ | ++++ |
| 113 | +++ | ++ | ++ | +++ |
| 118 | ++++ | ++ | +++ | ++++ |
| 127 | ++++ | + | +++ | +++ |
| 133 | ++ | ++ | ++ | ++ |
| 136 | +++++ | +++ | ++++ | ++++ |
| 137 | +++++ | ++ | +++ | ++++ |
| 154 | +++++ | ++ | ++++ | +++ |
| 156 | ++++ | + | +++ | +++ |
| 176 | + | + | ++ | ++ |
| 207 | +++++ | ++ | ++++ | +++ |
| 216 | +++++ | ++ | ++++ | +++ |
| 225 | ++++ | +++ | ++++ | ++++ |
| 226 | +++++ | +++ | ++++ | ++++ |
| 237 | ++++ | ++ | ++++ | +++ |
| 249 | +++++ | ++ | ++++ | ++++ |
| 294 | + | +++ | + | + |
| 295 | ++ | ++ | ++ | ++ |
| 296 | ++++ | ++ | +++ | ++ |
| 297 | ++++ | ++ | ++++ | ++ |
| 298 | +++ | +++ | ++ | ++ |
| 299 | + | ++ | ++ | +++ |
| 300 | ++++ | +++ | ++++ | ++++ |
| 301 | ++ | ++ | ++ | +++ |
| 302 | ++++ | +++ | +++ | +++ |
| 303 | ++ | +++ | ++ | + |
| 304 | ++++ | ++ | +++ | +++ |
| 305 | ++++ | ++ | +++ | +++ |
| 306 | ++++ | ++ | +++ | +++ |
| 307 | + | +++ | + | ++ |
| 308 | +++ | ++ | +++ | +++ |
| 309 | + | +++ | + | + |
| REF-1 | + | ++ | + | + |

### EQUIVALENTS

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the disclosure to the precise form disclosed, but by the claims appended hereto.

### Numbered Embodiments

1. A compound of Formula (I**):

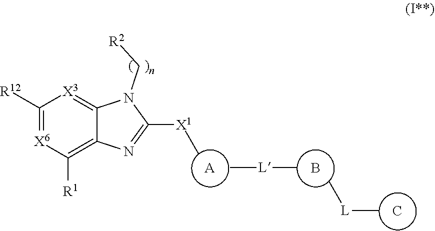

or a pharmaceutically acceptable salt thereof; wherein:
X³ is CR⁶ or N;
X⁶ is CR⁴ or N;
R¹ is —C₁₋₆ haloalkyl, halogen, —O—X⁴, or —NR⁸R⁹, or R¹ and R⁴, together with the atoms to which they are attached, combine to form a 5- or 6-membered heterocyclyl;
X⁴ is hydrogen, C₁₋₆ alkyl, C₁₋₆ heteroalkyl, C₁₋₆ haloalkyl, —(C₁₋₆ alkyl)-(C₃₋₁₀ cycloalkyl), or —(C₁₋₆ alkyl)-(3- to 8-membered heterocyclyl), 3- to 8-membered heterocyclyl, —(CH₂CH₂—O)₁₋₅—CH₃, —(CH₂—CH(—OCH₃)—CH₂—O)₁₋₅—CH₃, C₃₋₁₀ cycloalkyl, or C₆₋₁₀ aryl, wherein the alkyl, heteroalkyl, or haloalkyl group is optionally substituted with one or more deuterium, C₁₋₆ alkoxy, hydroxyl, —CN, or oxo, and the cycloalkyl, heterocyclyl, or aryl group is optionally substituted with one or more halogen, C₁₋₆ alkoxy, or —CN;
R⁶ is hydrogen, halogen, or —O—R⁷;
   wherein R⁷ and R², together with the atoms to which they are attached, combine to form a 6-membered heterocyclyl;
R⁸ and R⁹ each independently are selected from hydrogen, C₁₋₆ alkyl, or —S(O)₂—C₁₋₆ alkyl, or R⁸ and R⁹, together with the atoms to which they are attached, combine to form a 6-membered heterocycyl; wherein the C₁₋₆ alkyl is optionally substituted by one or more oxo;
n is 0, 1, 2, 3, 4, 5, or 6;
R² is:
   hydrogen;
   C₁₋₆ alkyl optionally substituted with deuterium; C₁₋₆ haloalkyl;
   —(O)—C₁₋₆ alkyl;
   —CN;
   a C₃₋₁₀ cycloalkyl optionally substituted with one or more —CN, C₁₋₆ haloalkyl, or C₁₋₆ alkyl optionally substituted with one or more —CN;
   a 4 or 5-membered heterocyclyl comprising at least one oxygen or at least one sulfur atom, wherein the 4- or 5-membered heterocyclyl is optionally substituted with one or more oxo,

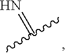 [SMILES: *=N]

or C₁₋₆ alkyl;
a 5-membered heteroaryl, comprising 1 or 2 heteroatoms independently selected from N, and S, wherein at least one heteroatom of R⁵ is S; or
R² and R⁷, together with the atoms to which they are attached, combine to form a 5- or 6-membered heterocyclyl;
R⁴ is halogen, hydrogen, —C(O)OH, or —O—R⁸,
   wherein R⁸ and R¹, together with the atoms to which they are attached, combine to form a 6-membered heterocyclyl;
R¹² is hydrogen, —C(O)OH, —C(O)NR^{N12}R^{N12′}, —C(O)NR¹²S(O)₂R¹², —(C₂₋₆ alkynylene)-C(O)OH, —(C₁₋₆ alkylene)-C(O) OH, —NR^{N12}—(C₁₋₆ alkylene)-C(O) OH, 5-10 membered heteroaryl or 5- to 10-membered heterocyclyl optionally substituted with one or more oxo, C₁₋₆ alkyl or C₁₋₆ haloalkyl;
R^{N12} and R^{N12′} independently are H or C₁₋₆ alkyl;
X¹ is

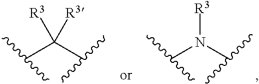

wherein R³ and R^{3′} independently are H, D or C₁₋₆ alkyl, wherein the C₁₋₆ alkyl is optionally substituted with deuterium;
Ring A is

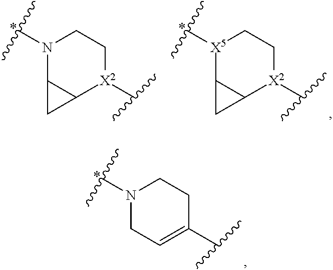

phenyl optionally substituted with one or more halo or C₁₋₆ alkyl, or 6-membered heteroaryl optionally substituted with one or more halo or C₁₋₆ alkyl;
wherein * indicates attachment to X¹,
   X⁵ is CR³ or N, and
   X² is CR³ or N;
L′ is a bond or —O—;
Ring B is a C₆₋₁₀ arylene, a 5-10 membered heteroarylene, or a 3-10 membered heterocycylene, wherein the C₆₋₁₀ arylene, 5-10 membered heteroarylene, or 3-10 membered heterocyclene is optionally substituted with one or more oxo, C₁₋₆ alkyl, C₁₋₆ alkoxy, or halogen;
L is a bond, *—(C₁₋₆ alkylene)-, *—NR^{L}—(C₁₋₆ alkyl), *—O—(C₁₋₆ alkyl)-, or *—(C₁₋₆ alkyl)-O—, wherein * indicates attachment to Ring B and the C₁₋₆ alkylene or C₁₋₆ alkyl is optionally substituted with deuterium;
   wherein R^{L} is H or C₁₋₆ alkyl; and
Ring C is:
a 6-membered aryl optionally substituted with one or more C₃₋₁₀ cycloalkyl, C₁₋₆ alkyl, C₁₋₆ haloalkyl, 3-10 membered heterocyclyl, halogen, C₁₋₆ alkoxy, C₁₋₆ haloalkoxy, —CN, C₃₋₁₀ cycloalkyl, or —C(O)NR′₂;
   wherein R′ is H or C₁₋₆ alkyl
a 6-membered heteroaryl comprising a nitrogen atom optionally substituted with one or more halogen, —CN, C₁₋₆ haloalkyl, —O—C₁₋₆ alkyl, C₃₋₁₀ cycloalkyl, —C(═O)—(C₃₋₁₀ cycloalkyl),

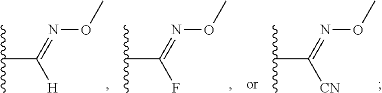

or
a bicylic 9- or 10-membered heteroaryl or heterocyclyl optionally substituted with one or more C₁₋₆ alkyl, halogen, —CN, or oxo.

2. The compound of embodiment 1, wherein the compound of Formula (I**) is of Formula I*:

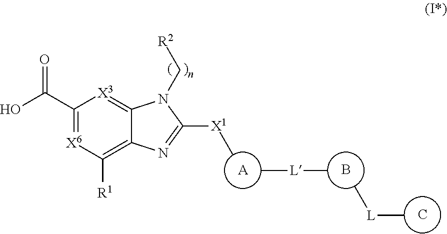

or a pharmaceutically acceptable salt thereof; wherein:
X³ is CR⁶ or N;
X⁶ is CR⁴ or N;
R¹ is hydrogen, —C₁₋₆ haloalkyl, —O—X⁴, or —NR⁸R⁹, or R¹ and R⁴, together with the atoms to which they are attached, combine to form a 6-membered heterocyclyl;
X⁴ is hydrogen, C₁₋₆ alkyl, C₁₋₆ heteroalkyl, C₁₋₆ haloalkyl, —(C₁₋₆ alkyl)-(C₃₋₁₀ cycloalkyl), or —(C₁₋₆ alkyl)-(3- to 8-membered heterocyclyl), 3- to 8-membered heterocyclyl, —(CH₂CH₂—O)₁₋₅—CH₃, or —(CH₂—CH(—OCH₃)—CH₂—O)₁₋₅—CH₃, wherein the alkyl, heteroalkyl, or haloalkyl group is optionally substituted with one or more deuterium, hydroxyl, —CN, or oxo, and the cycloalkyl or heterocyclyl group is optionally substituted with one or more halogen or —CN;
R⁶ is hydrogen, halogen, or —O—R⁷;
   wherein R⁷ is hydrogen, or R⁷ and R², together with the atoms to which they are attached, combine to form a 6-membered heterocyclyl;
R⁸ and R⁹ each independently are selected from hydrogen, C₁₋₆ alkyl, or —S(O)₂—C₁₋₆ alkyl, or R⁸ and R⁹, together with the atoms to which they are attached, combine to form a 6-membered heterocycyl; wherein the C₁₋₆ alkyl is optionally substituted by one or more oxo;
n is 0, 1, 2, 3, 4, 5, or 6;
R² is:
hydrogen;
branched C₂₋₆ alkyl;
C₁₋₆ haloalkyl;
—(O)—C₁₋₆ alkyl;
a C₃₋₁₀ cycloalkyl optionally substituted with one or more —CN, C₁₋₆ haloalkyl, or C₁₋₆ alkyl optionally substituted with one or more —CN;
a 4 or 5-membered heterocyclyl comprising at least one oxygen or at least one sulfur atom, wherein the 4- or 5-membered heterocyclyl is optionally substituted with one or more oxo,

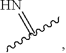 [SMILES: *=N]

or C₁₋₆ alkyl;
a 5-membered heteroaryl, comprising 1 or 2 heteroatoms independently selected from N, and S, wherein at least one heteroatom of R⁵ is S; or
R² and R⁷, together with the atoms to which they are attached, combine to form a 5- or 6-membered heterocyclyl;
R⁴ is hydrogen or —O—R⁸,
   wherein R⁸ and R¹, together with the atoms to which they are attached, combine to form a 6-membered heterocyclyl;
X¹ is

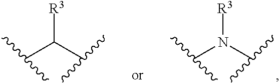

wherein R³ is H or C₁₋₆ alkyl;
Ring A is

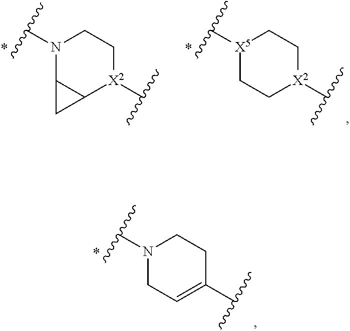

phenyl optionally substituted with one or more halo or C₁₋₆ alkyl, or 6-membered heteroaryl optionally substituted with one or more halo or C₁₋₆ alkyl;
wherein * indicates attachment to X¹,
   X⁵ is CR³ or N, and
   X² is CR³ or N;
L′ is a bond or —O—;
Ring B is a C₆₋₁₀ arylene, a 6-10 membered heteroarylene, or a 3-10 membered heterocycylene, wherein the C₆₋₁₀ arylene, 6-10 heteroarylene, or 3-10 membered heterocyclene is optionally substituted with one or more C₁₋₆ alkyl, C₁₋₆ alkoxy, or halogen;
L is a bond, *—CH₂—, *—O—(C₁₋₆ alkyl)-, or *—(C₁₋₆ alkyl)-O—, wherein * indicates attachment to Ring B and the C₁₋₆ alkyl is optionally substituted with deuterium or C₁₋₆ alkyl; and
Ring C is:
a 6-membered aryl optionally substituted with one or more C₁₋₆ alkyl, 3-10 membered heterocyclyl, halogen, —OCH₃, —CN, or C₃₋₁₀ cycloalkyl;
a 6-membered heteroaryl comprising a nitrogen atom optionally substituted with one or more halogen, —CN, —O—C₁₋₆ alkyl, C₃₋₁₀ cycloalkyl, —C(═O)—(C₃₋₁₀ cycloalkyl),

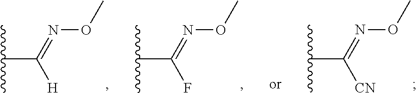

a bicylic 9- or 10-membered heteroaryl comprising two nitrogen atoms optionally substituted with one or more C₁₋₆ alkyl, or oxo; or
a bicyclic 10-membered heterocyclyl comprising one or more oxygen atoms;
with the proviso that when R¹ is hydrogen, then X³ is CR⁶, wherein R⁶ is —OH.

3. The compound of embodiment 1, wherein the compound of Formula I* is of Formula I″:

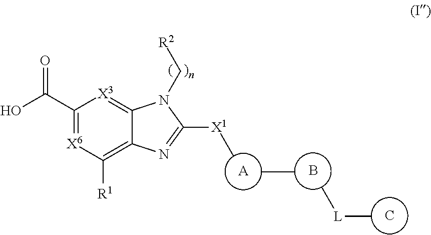

or a pharmaceutically acceptable salt thereof; wherein:
X³ is CR⁶ or N;
X⁶ is CR⁴ or N;
R¹ is —C₁₋₆ haloalkyl, —O—X⁴, or —NR⁸R⁹, or R¹ and R⁴, together with the atoms to which they are attached, combine to form a 6-membered heterocyclyl;
wherein X⁴ is hydrogen, C₁₋₆ alkyl, C₁₋₆ heteroalkyl, C₁₋₆ haloalkyl, —(C₁₋₆ alkyl)-(C₃₋₁₀ cycloalkyl), or —(C₁₋₆ alkyl)-(3- to 8-membered heterocyclyl), 3- to 8-membered heterocyclyl, —(CH₂CH₂—O)₁₋₅—CH₃, or —(CH₂—CH(—OCH₃)—CH₂—O)₁₋₅—CH₃, wherein the alkyl, heteroalkyl, or haloalkyl group is optionally substituted with one or more deuterium, hydroxyl, —CN, or oxo, and the cycloalkyl or heterocyclyl group is optionally substituted with one or more halogen or —CN;
R⁶ is hydrogen, or —O—R⁷;
   wherein R⁷ and R², together with the atoms to which they are attached, combine to form a 5- or 6-membered heterocyclyl;
R⁸ and R⁹ each independently are selected from hydrogen, C₁₋₆ alkyl, or —S(O)₂—C₁₋₆ alkyl, or R⁸ and R⁹, together with the atoms to which they are attached, combine to form a 6-membered heterocycyl; wherein the C₁₋₆ alkyl is optionally substituted by one or more oxo;
n is 0, 1, 2, 3, 4, 5, or 6;
R² is:
hydrogen;
branched C₂₋₆ alkyl;
C₁₋₆ haloalkyl;
—(O)—C₁₋₆ alkyl;
a C₃₋₁₀ cycloalkyl optionally substituted with one or more —CN, C₁₋₆ haloalkyl, or C₁₋₆ alkyl optionally substituted with one or more —CN;
a 4 or 5-membered heterocyclyl comprising at least one oxygen or at least one sulfur atom, wherein the 4- or 5-membered heterocyclyl is optionally substituted with one or more oxo,

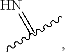 [SMILES: *=N]

or C₁₋₆ alkyl;
a 5-membered heteroaryl, comprising 1 or 2 heteroatoms independently selected from N, and S, wherein at least one heteroatom of R⁵ is S; or
R² and R⁷, together with the atoms to which they are attached, combine to form a C₂₋₉ heterocyclyl;
R⁴ is hydrogen or —O—R⁸,
   wherein R⁸ and R¹, together with the atoms to which they are attached, combine to form a 6-membered heterocyclyl; X¹ is

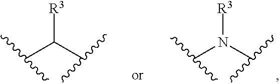

wherein R³ is H or C₁₋₆ alkyl;
Ring A is

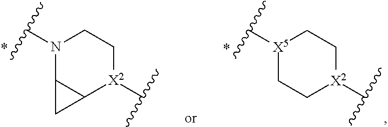

wherein * indicates attachment to X¹,
   X⁵ is CH or N, and
   X² is CH or N;
Ring B is a C₆₋₁₀ arylene, a 6-10 membered heteroarylene, or a 3-10 membered heterocycylene, wherein the C₆₋₁₀ arylene, 6-10 heteroarylene, or 3-10 membered heterocyclene is optionally substituted with one or more C₁₋₆ alkyl, C₁₋₆ alkoxy, or halogen;
L is a bond, *—CH₂—, *—O—(C₁₋₆ alkyl)-, or *—(C₁₋₆ alkyl)-O—, wherein * indicates attachment to Ring B and the C₁₋₆ alkyl is optionally substituted with deuterium; and
Ring C is:
a 6-membered aryl optionally substituted with one or more C₁₋₆ alkyl, 3-10 membered heterocyclyl, halogen, —OCH₃, —CN, or C₃₋₁₀ cycloalkyl;
a 6-membered heteroaryl comprising a nitrogen atom optionally substituted with one or more halogen, —CN, —O—C₁₋₆ alkyl, C₃₋₁₀ cycloalkyl, —C(═O)—(C₃₋₁₀ cycloalkyl),

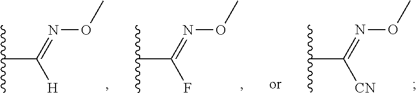

or
a bicylic 9- or 10-membered heteroaryl comprising two nitrogen atoms optionally substituted with one or more C₁₋₆ alkyl, or oxo.

4. A compound of Formula I′″*:

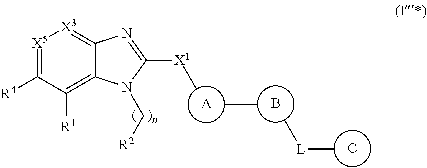

or a pharmaceutically acceptable salt thereof; wherein:
X³ is CR^{X3} or N;
   wherein R^{X3} is H, halogen, C₁₋₆ alkyl, C₁₋₆ haloalkoxy, —O—(CH₂CH₂—O)₁₋₅—CH₃, or C₁₋₆ alkoxy;
X^{5′} is CR^{5′}
R¹ is —C₁₋₆ haloalkyl or —O—X⁴, or R¹ and R⁴, together with the atoms to which they are attached, combine to form a 6-membered heterocyclyl;
wherein X⁴ is hydrogen, C₁₋₆ alkyl, C₁₋₆ heteroalkyl, C₁₋₆ haloalkyl, —(C₁₋₆ alkyl)-(C₃₋₁₀ cycloalkyl), or —(C₁₋₆ alkyl)-(3- to 8-membered heterocyclyl), 3- to 8-membered heterocyclyl, —(CH₂CH₂—O)₁₋₅—CH₃, or —(CH₂—CH(—OCH₃)—CH₂—O)₁₋₅—CH₃, wherein the alkyl, heteroalkyl, or haloalkyl group is optionally substituted with one or more deuterium, halogen, or —CN, and the cycloalkyl or heterocyclyl group is optionally substituted with one or more halogen or —CN;
n is 0, 1, 2, 3, 4, 5, or 6;
R² is:
   hydrogen;
   halogen;
   branched C₃₋₆ alkyl;
   —(O)—C₁₋₆ alkyl;
   C₃₋₁₀ cycloalkyl optionally substituted with one or more —CN, C₁₋₆ haloalkyl, or C₁₋₆ alkyl optionally substituted with one or more —CN;
   4 or 5-membered heterocyclyl comprising at least one oxygen or at least one sulfur atom, wherein the 4- or 5-membered heterocyclyl is optionally substituted with one or more oxo,

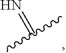 [SMILES: *=N]

or C₁₋₆ alkyl;
5-membered heteroaryl, comprising 1 or 2 heteroatoms independently selected from N, and S, wherein at least one heteroatom of R⁵ is S; or
R² and R¹, together with the atoms to which they are attached, combine to form a 6-membered heterocyclyl;
R^{5′} is hydrogen or —C(O)—OH;
R⁴ is hydrogen, halogen, —C(O)—OH, or —O—R⁸,
   wherein R⁸ and R¹, together with the atoms to which they are attached, combine to form a 6-membered heterocyclyl; X¹ is

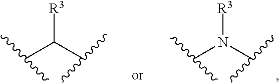

wherein R³ is H or C₁₋₆ alkyl;

### Ring A is

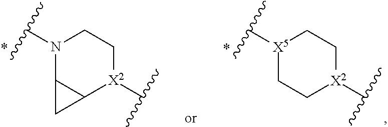 [7203] wherein * indicates attachment to X¹,
wherein X⁵ is CH or N, and
   X² is CH or N;
Ring B is a 6-10 membered heteroarylene, or a 3-10 membered heterocycylene optionally substituted with one or more C₁₋₆ alkyl;
L is a bond or *—O—(C₁₋₆ alkyl)-, wherein * indicates attachment to Ring B;
Ring C is:
a 6-membered aryl optionally substituted with one or more halogen, —OCH₃, —CN, or C₃₋₁₀ cycloalkyl;
a 6-membered heteroaryl comprising a nitrogen atom optionally substituted with one or more halogen, —CN, —O—C₁₋₆ alkyl, C₃₋₁₀ cycloalkyl, —C(═O)—(C₃₋₁₀ cycloalkyl),

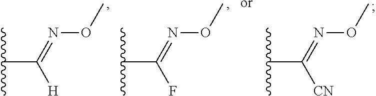

or
a bicylic 9- or 10-membered heteroaryl comprising two nitrogen atoms optionally substituted with one or more C₁₋₆ alkyl, or oxo.

5. The compound of embodiment 4, wherein the compound of Formula I′″* is of Formula I′″

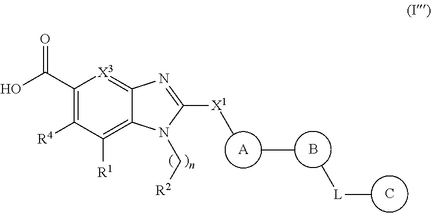

or a pharmaceutically acceptable salt thereof; wherein:
X³ is CR^{X3} or N;
   wherein R^{X3} is H, halogen, C₁₋₆ alkyl, C₁₋₆ haloalkoxy, or C₁₋₆ alkoxy;
R¹ is —C₁₋₆ haloalkyl or —O—X⁴, or R¹ and R⁴, together with the atoms to which they are attached, combine to form a 6-membered heterocyclyl;
wherein X⁴ is hydrogen, C₁₋₆ alkyl, C₁₋₆ heteroalkyl, C₁₋₆ haloalkyl, —(C₁₋₆ alkyl)-(C₃₋₁₀ cycloalkyl), or —(C₁₋₆ alkyl)-(3- to 8-membered heterocyclyl), 3- to 8-membered heterocyclyl, —(CH₂CH₂—O)₁₋₅—CH₃, or —(CH₂—CH(—OCH₃)—CH₂—O)₁₋₅—CH₃, wherein the alkyl, heteroalkyl, or haloalkyl group is optionally substituted with one or more deuterium, —CN, and the cycloalkyl or heterocyclyl group is optionally substituted with one or more halogen or —CN;
n is 0, 1, 2, 3, 4, 5, or 6;
R² is:
   hydrogen;
   halogen;
   C₃₋₆ alkyl;
   —(O)—C₁₋₆ alkyl;
   a C₃₋₁₀ cycloalkyl optionally substituted with one or more —CN, C₁₋₆ haloalkyl, or C₁₋₆ alkyl optionally substituted with one or more —CN;
   a 4 or 5-membered heterocyclyl comprising at least one oxygen or at least one sulfur atom, wherein the 4- or 5-membered heterocyclyl is optionally substituted with one or more oxo,

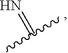 [SMILES: *=N ; ","]

or C₁₋₆ alkyl;
a 5-membered heteroaryl, comprising 1 or 2 heteroatoms independently selected from N, and S, wherein at least one heteroatom of R⁵ is S; or
R² and R¹, together with the atoms to which they are attached, combine to form a 6-membered heterocyclyl;
R⁴ is hydrogen or —O—R⁸,
   wherein R⁸ and R¹, together with the atoms to which they are attached, combine to form a 6-membered heterocyclyl;
X¹ is

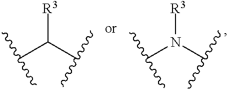

wherein R³ is H or C₁₋₆ alkyl;

### Ring A is

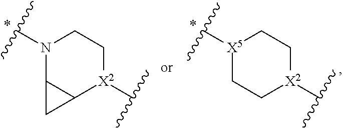 [7232] wherein * indicates attachment to X¹,
wherein X⁵ is CH or N, and
   X² is CH or N;
Ring B is a 6-10 membered heteroarylene, or a 3-10 membered heterocycylene optionally substituted with one or more C₁₋₆ alkyl;
L is a bond or *—O—(C₁₋₆ alkyl)-, wherein * indicates attachment to Ring B;
Ring C is:
a 6-membered aryl optionally substituted with one or more halogen, —OCH₃, —CN, or C₃₋₁₀ cycloalkyl;
a 6-membered heteroaryl comprising a nitrogen atom optionally substituted with one or more halogen, —CN, —O—C₁₋₆ alkyl, C₃₋₁₀ cycloalkyl, —C(═O)—(C₃₋₁₀ cycloalkyl),

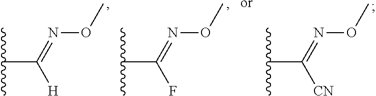

or
a bicylic 9- or 10-membered heteroaryl comprising two nitrogen atoms optionally substituted with one or more C₁₋₆ alkyl, or oxo.

6 The compound of any one of the preceding embodiments, wherein, the compound of Formula I** is of Formula I′

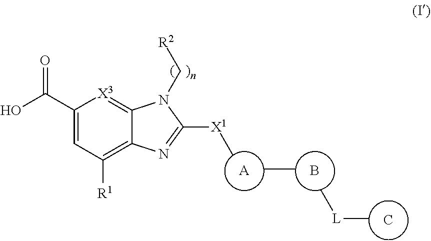

or a pharmaceutically acceptable salt thereof; wherein:
X³ is CH or N;
R¹ is —C₁₋₆ haloalkyl or —O—X⁴;
wherein X⁴ is hydrogen, C₁₋₆ alkyl, C₁₋₆ heteroalkyl, C₁₋₆ haloalkyl, —(C₁₋₆ alkyl)-(C₃₋₁₀ cycloalkyl), —(C₁₋₆ alkyl)-(3- to 8-membered heterocyclyl), 3- to 8-membered heterocyclyl, —(CH₂CH₂—O)₁₋₅—CH₃, or —(CH₂—CH(—OCH₃)—CH₂—O)₁₋₅—CH₃, wherein the alkyl, heteroalkyl, or haloalkyl group is optionally substituted with one or more —CN, and the cycloalkyl or heterocyclyl group is optionally substituted with one or more halogen or —CN;
n is 0, 1, 2, 3, 4, 5, or 6;
R² is:
hydrogen;
(O)—C₁₋₆ alkyl;
a C₃₋₁₀ cycloalkyl optionally substituted with one or more —CN, C₁₋₆ haloalkyl, or C₁₋₆ alkyl optionally substituted with one or more —CN;
a 4 or 5-membered heterocyclyl comprising at least one oxygen or at least one sulfur atom, wherein the 4- or 5-membered heterocyclyl is optionally substituted with one or more oxo,

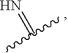 [SMILES: *=N ; ","]

or C₁₋₆ alkyl; or
a 5-membered heteroaryl, comprising 1 or 2 heteroatoms independently selected from N, and S, wherein at least one heteroatom of R⁵ is S;
X¹ is

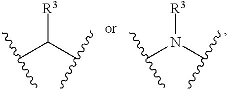

wherein R³ is H or C₁₋₆ alkyl;

### Ring A is

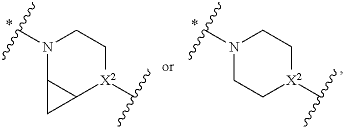 [7255] wherein * indicates attachment to X¹, and
wherein X² is CH or N;
Ring B is a 6-10 membered heteroarylene, or a 3-10 membered heterocycylene optionally substituted with one or more C₁₋₆ alkyl;
L is a bond or *—O—(C₁₋₆ alkyl)-, wherein * indicates attachment to Ring B;
Ring C is:
a 6-membered aryl optionally substituted with one or more halogen, —OCH₃, —CN, or C₃₋₁₀ cycloalkyl;
a 6-membered heteroaryl comprising a nitrogen atom optionally substituted with one or more halogen, —CN, —O—C₁₋₆ alkyl, C₃₋₁₀ cycloalkyl, —C(═O)—(C₃₋₁₀ cycloalkyl),

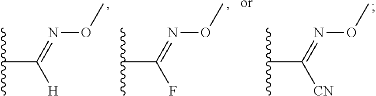

a bicylic 9- or 10-membered heteroaryl comprising two nitrogen atoms optionally substituted with one or more C₁₋₆ alkyl, or oxo.

7. The compound of any one of the preceding embodiments, wherein the compound of Formula (I**) is of Formula (I):

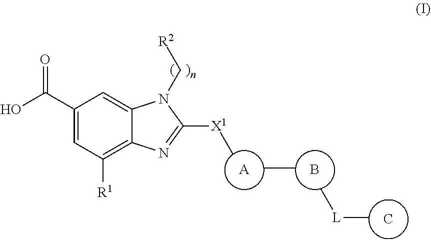

or a pharmaceutically acceptable salt thereof; wherein
is —C₁₋₆ haloalkyl or —O—X⁴;
wherein X⁴ is hydrogen, C₁₋₆ alkyl, C₁₋₆ heteroalkyl, C₁₋₆ haloalkyl, —(C₁₋₆ alkyl)-(C₃₋₁₀ cycloalkyl), or —(C₁₋₆ alkyl)-(3- to 8-membered heterocyclyl), —(—CH₂CH₂—O)₁₋₅—CH₃, or —(CH₂—CH(—OCH₃)—CH₂—O)₁₋₅—CH₃, wherein the alkyl, heteroalkyl, or haloalkyl group is optionally substituted with one or more —CN, and the cycloalkyl or heterocyclyl group is optionally substituted with one or more halogen or —CN;
n is 0, 1, 2, 3, 4, 5, or 6;
R² is:
hydrogen;
—(O)—C₁₋₆ alkyl;
a C₃₋₁₀ cycloalkyl optionally substituted with one or more —CN, C₁₋₆ haloalkyl, or C₁₋₆ alkyl optionally substituted with one or more —CN;
a 4 or 5-membered heterocyclyl comprising at least one oxygen or at least one sulfur atom, wherein the 4- or 5-membered heterocyclyl is optionally substituted with one or more oxo,

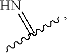 [SMILES: *=N ; ","]

or C₁₋₆ alkyl; or
a 5-membered heteroaryl, comprising 1 or 2 heteroatoms independently selected from N, and S, wherein at least one heteroatom of R⁵ is S;
X¹ is

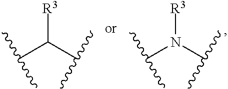

wherein R³ is H or C₁₋₆ alkyl;

### Ring A is

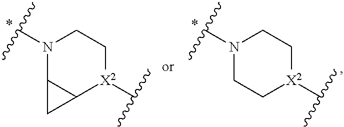 [7276] wherein * indicates attachment to X¹, and
wherein X² is CH or N;
Ring B is:
a 6-membered heteroarylene comprising nitrogen;
a 9-membered heterocycylene comprising two oxygen atoms optionally substituted with one or more C₁₋₆ alkyl; or
a 10-membered heterocycylene comprising two oxygen atoms;
L is a bond or *—O—(C₁₋₆ alkyl)-, wherein * indicates attachment to Ring B;
Ring C is:
a 6-membered aryl optionally substituted with one or more halogen, —OCH₃, —CN, or C₃₋₁₀ cycloalkyl;
a 6-membered heteroaryl comprising a nitrogen atom optionally substituted with one or more halogen, —CN, —O—C₁₋₆ alkyl, C₃₋₁₀ cycloalkyl, —C(═O)—(C₃₋₁₀ cycloalkyl),

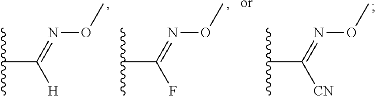

a bicylic 9- or 10-membered heteroaryl comprising two nitrogen atoms optionally substituted with one or more C₁₋₆ alkyl, or oxo.

8. The compound of any one of the preceding embodiments, wherein the compound of Formula (I**) is of Formula (Ia):

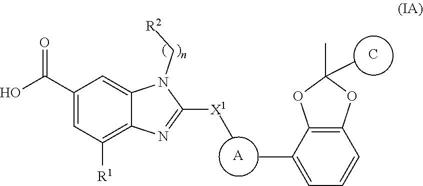

or a pharmaceutically acceptable salt thereof.

9. The compound of any one of the preceding embodiments, wherein the compound of Formula (I**) is of Formula (IB):

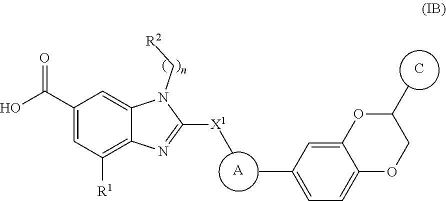

or a pharmaceutically acceptable salt thereof.

10. The compound of any one of the preceding embodiments, wherein the compound of Formula (I**) is of Formula (IC):

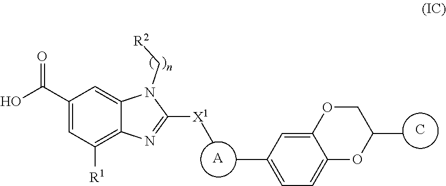

or a pharmaceutically acceptable salt thereof.

11. The compound of any one of the preceding embodiments, wherein the compound of Formula (I**) is of Formula (ID):

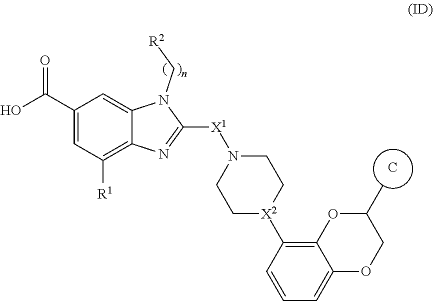

or a pharmaceutically acceptable salt thereof.

12. The compound of any one of the preceding embodiments, wherein the compound of Formula (I**) is of Formula (IE):

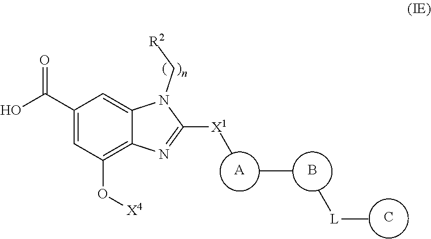

or a pharmaceutically acceptable salt thereof.

13. The compound of any one of the preceding embodiments, wherein the compound of Formula (I**) is of Formula (IF):

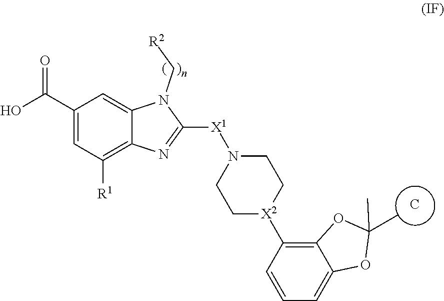

or a pharmaceutically acceptable salt thereof.

14. The compound of any one of the preceding embodiments, wherein the compound of Formula (I**) is of Formula (IG):

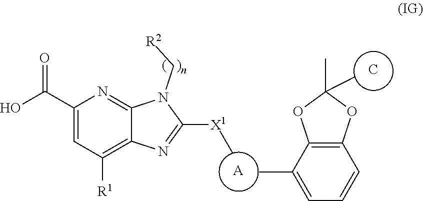

or a pharmaceutically acceptable salt thereof.

15. The compound of any one of the preceding embodiments, wherein the compound of Formula (I**) is of Formula (IH):

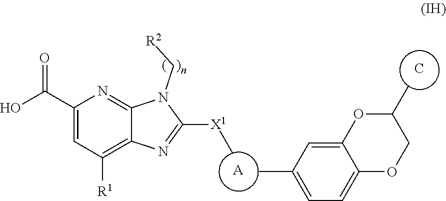

or a pharmaceutically acceptable salt thereof.

16. The compound of any one of the preceding embodiments, wherein the compound of Formula (I**) is of Formula (II):

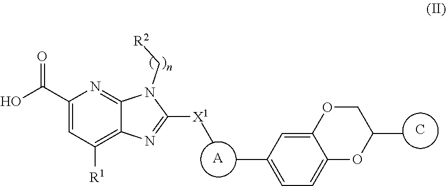

or a pharmaceutically acceptable salt thereof.

17. The compound of any one of the preceding embodiments, wherein the compound of Formula (I**) is of Formula (IJ):

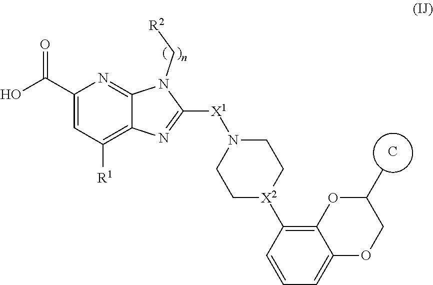

or a pharmaceutically acceptable salt thereof.

18. The compound of any one of the preceding embodiments, wherein the compound of Formula (I**) is of Formula (IK):

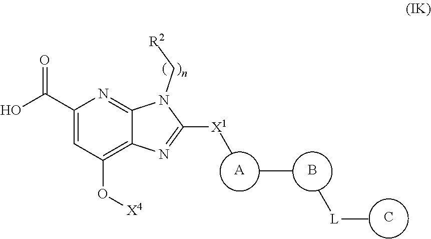

or a pharmaceutically acceptable salt thereof.

19. The compound of any one of the preceding embodiments, wherein the compound of Formula (I**) is of Formula (IL):

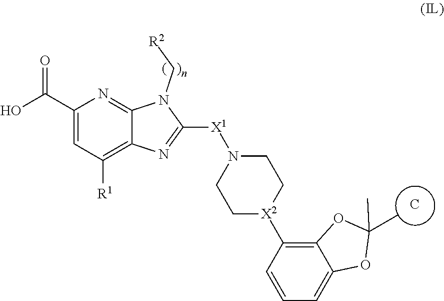

or a pharmaceutically acceptable salt thereof.

20. The compound of any one of the preceding embodiments, wherein the compound of Formula (I**) is of Formula (IM):

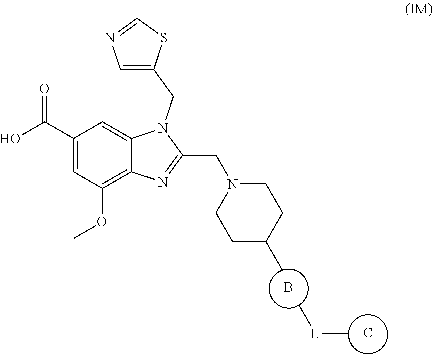

or a pharmaceutically acceptable salt thereof.

21. The compound of any one of the preceding embodiments, wherein the compound of Formula (I**) is of Formula (IN):

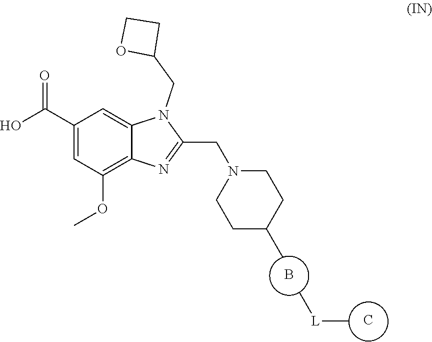

or a pharmaceutically acceptable salt thereof.

22. The compound of any one of the preceding embodiments, wherein the compound of Formula (I**) is of Formula (IO):

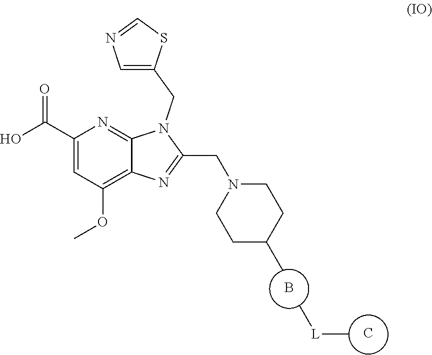

or a pharmaceutically acceptable salt thereof.

23. The compound of any one of the preceding embodiments, wherein the compound of Formula (I**) is of Formula (IP):

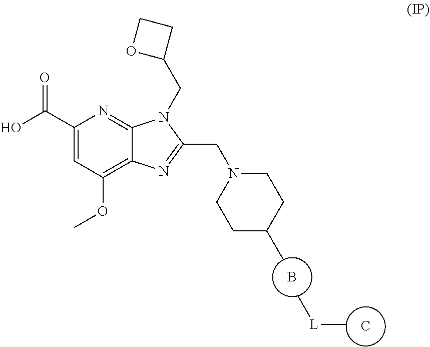

or a pharmaceutically acceptable salt thereof.

24. The compound of any one of the preceding embodmients, wherein the compound of Formula (I**) is of Formula (IQ):

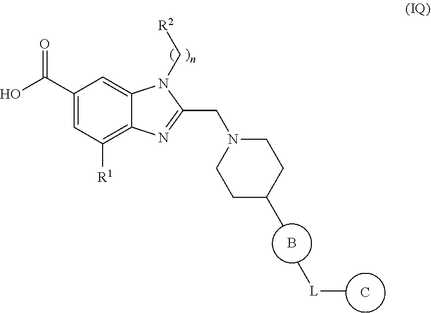

or a pharmaceutically acceptable salt thereof.

25. The compound of any one of the preceding embodiments, wherein the compound of Formula (I**) is of Formula (IR):

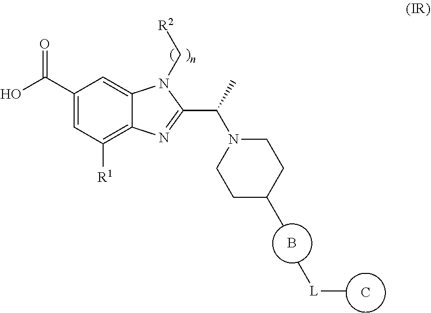

or a pharmaceutically acceptable salt thereof.

26. The compound of any one of the preceding embodiments, wherein the compound of Formula (I**) is of Formula (IS):

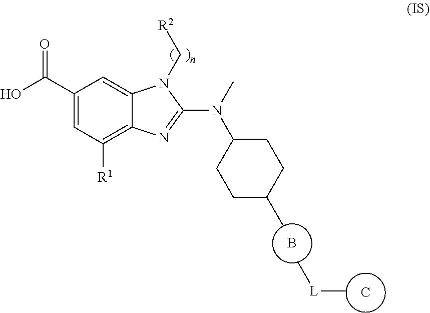

or a pharmaceutically acceptable salt thereof.

27. The compound of any one of the preceding embodiments, wherein the compound of Formula (I**) is of Formula (IT):

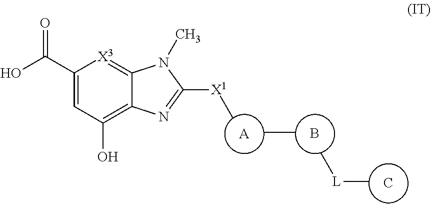

or a pharmaceutically acceptable salt thereof.

28. The compound of any one of the preceding embodiments, wherein the compound of Formula (I**) is of Formula (IU):

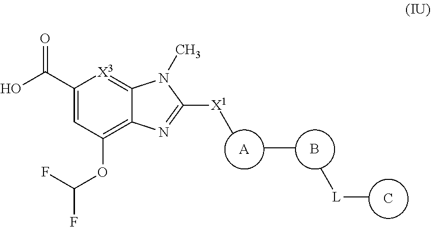

or a pharmaceutically acceptable salt thereof.

29. The compound of any one of the preceding embodiments, wherein the compound of Formula (I**) is of Formula (IX):

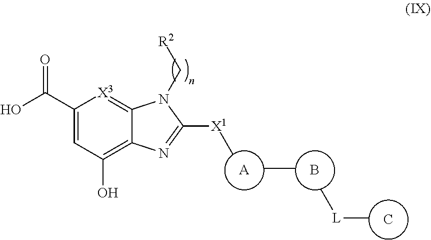

or a pharmaceutically acceptable salt thereof.

30. The compound of any one of the preceding embodiments, wherein the compound of Formula (I**) is of Formula (IY):

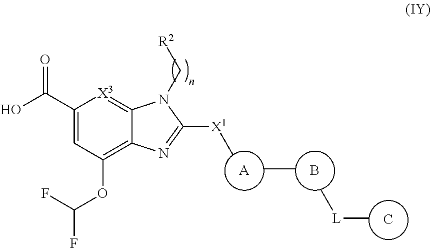

or a pharmaceutically acceptable salt thereof.

31. The compound of any one of the preceding embodiments, wherein the compound of Formula (I**) is of Formula (IZ):

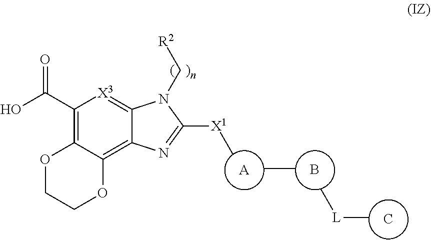

or a pharmaceutically acceptable salt thereof.

32. The compound of any one of the preceding embodiments, wherein the compound of Formula (I**) is of Formula (IAA):

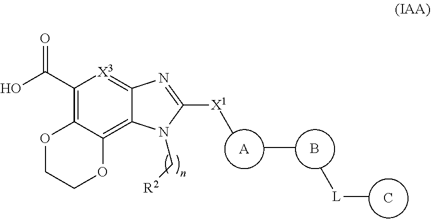

or a pharmaceutically acceptable salt thereof.

33. The compound of any one of the preceding embodiments, wherein the compound of Formula (I**) is of Formula (IAB):

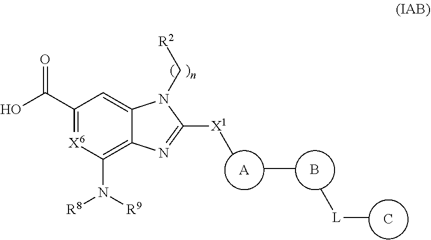

or a pharmaceutically acceptable salt thereof.

34. The compound of any one of the preceding embodiments, wherein the compound of Formula (I**) is of Formula (IAC):

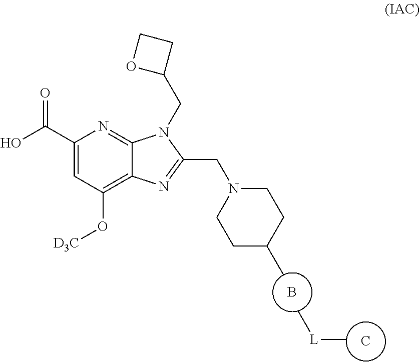

or a pharmaceutically acceptable salt thereof.

35. The compound of any one of the preceding embodiments, wherein the compound of Formula (I**) is of Formula (IAD):

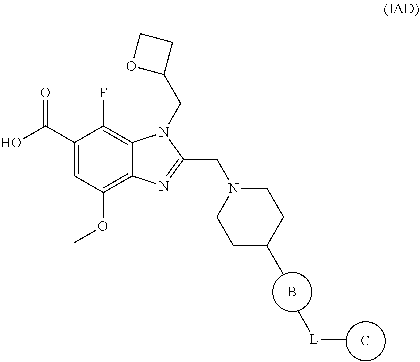

or a pharmaceutically acceptable salt thereof.

36. The compound of any one of the preceding embodiments, wherein the compound of Formula (I**) is of Formula (IAE):

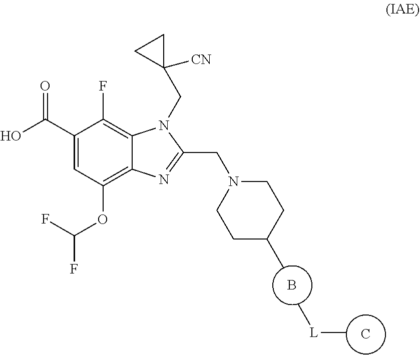

or a pharmaceutically acceptable salt thereof.

37. The compound of any one of the preceding embodiments, wherein the compound of Formula (I**) is of Formula (IAG):

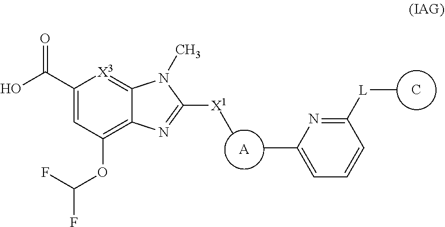

or a pharmaceutically acceptable salt thereof.

38. The compound of any one of the preceding embodiments, therein the compound of Formula (I**) is of Formula (IAH):

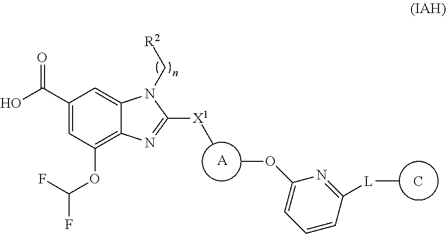

or a pharmaceutically acceptable salt thereof.

39. The compound of any one of the preceding embodiments, therein the compound of Formula (I**) is of Formula (IAI):

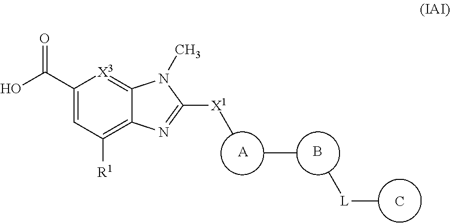

wherein R¹ is —O—(C₁₋₆ haloalkyl);
   or a pharmaceutically acceptable salt thereof.

40. The compound of any one of the preceding embodiments, wherein the compound of Formula (I**) is of Formula (IAJ):

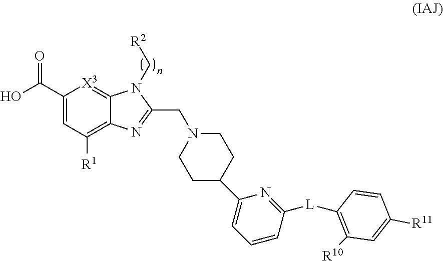

wherein R² is hydrogen, thiazolyl, oxetanyl, cyclopropyl optionally substituted with cyano, or methoxy;
n is 1 or 2;

R¹ is —O—C₁₋₆ alkyl, —OH, or —NH₂, wherein the C₁₋₆ alkyl is optionally substituted with one or more halo or deuterium;
X³ is CR⁶, wherein R⁶ is halo or hydrogen;
L is *—O—(C₁₋₆ alkyl)-, wherein * indicates attachment to Ring B and the C₁₋₆ alkyl is optionally substituted with deuterium;
R¹⁰ is halo; and
R¹¹ is halo or cyano;
   or a pharmaceutically acceptable salt thereof.

41. The compound of any one of the previous embodiments, wherein the compound of Formula (I**) is of Formula (II**)

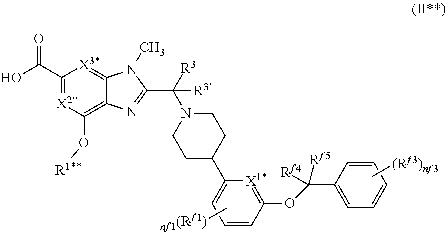

or a pharmaceutically acceptable salt thereof; wherein
R^{f4} and R^{f5} are each independently selected from C₁₋₆ alkyl, H and D
nf1 is 0, 1, 2, 3, or 4;
nf3 is 0, 1, 2, 3, 4, or 5;
each R^{f1} is halogen;
R³ and R^{3′} independently are H or D;
X¹* is N or CR^{f1};
X^{2*} and X^{3*} independently are CH or CF;
each R^{f3} is independently selected from halogen, —CN, C₁₋₆ alkyl, C₁₋₆ haloalkyl, C₁₋₆ alkoxy, or —C(O)N(R^{f3′})₂,
each R^{f3′} is independently selected from H or C₁₋₆ alkyl; and
R^{1**} is H or C₁₋₂ alkyl optionally substituted with one or more deuterium or halogen.

42. The compound of any one of the preceding embodiments, wherein the compound of Formula (I**) is of Formula (II*)

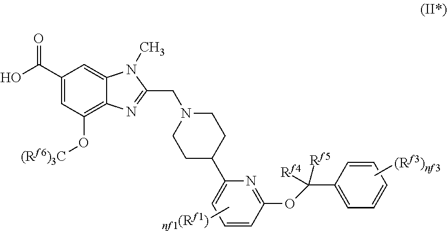

or a pharmaceutically acceptable salt thereof; wherein
R^{f4} and R^{f5} are each independently selected from C₁₋₆ alkyl, H and D
nf1 is 0, 1, 2, or 3;
nf3 is 0, 1, 2, 3, 4, or 5;
each R^{f1} is halogen;
each R^{f3} is independently selected from halogen, —CN, C₁₋₆ alkyl, C₁₋₆ haloalkyl, C₁₋₆ alkoxy, or —C(O)N(R^{f3′})₂,
   each R^{f3′} is independently selected from H or C₁₋₆ alkyl; and each R^{f6} is independently selected from H, D or F.

43. The compound of any one of the preceding embodiments, wherein R^{f4} and R^{f5} are each independently selected from CH₃, H, and D.

44. The compound of any one of the preceding embodiments, wherein each R^{f1} is fluorine.

45. The compound of any one of the preceding embodiments, wherein X³ is N.

46. The compound of any one of the preceding embodiments, wherein X³ is CH.

47. The compound of any one of the preceding embodiments, wherein X⁶ is N.

48. The compound of any one of the preceding embodiments, wherein X⁶ is CR⁴.

49. The compound of any one of the preceding embodiments, wherein R¹ is —O—C₁₋₆ alkyl, wherein the alkyl group is linear and is optionally substituted with one or more deuterium, —CN or —O—C₁₋₆ alkyl.

50. The compound of any one of the preceding embodiments, wherein R¹ is —O—CH₂—CH₃, —O—CH₃, —O—CH₂—CN, —O—CH₂—CH₂—O—CH₃, —O—CH₂CH(—O—CH₃)—CH₃, or —O—CD₃.

51. The compound of any one of the preceding embodiments, wherein R¹ is —O—C₁₋₆ alkyl, wherein the alkyl group is branched and is optionally substituted with one or more —CN.

52. The compound of any one of the preceding embodiments, wherein R¹ is

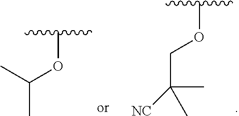

53. The compound of any one of the preceding embodiments, wherein R¹ is —O—C₁₋₆ haloalkyl.

54. The compound of any one of the preceding embodiments, wherein R¹ is —O—C₁₋₆ haloalkyl, wherein the haloalkyl group is linear.

55. The compound of any one of the preceding embodiments, wherein R¹ is —O—CHF₂, —O—CF₃, —O—CH₂—CH₂F, —O—CH₂—CF₃, —O—CH₂—CHF—CH₃, —O—CF₂—CH₃, —O—CHF—CH₃, —O—CHF—CH₂F, or —O—CH₂—CH₂F.

56. The compound of any one of the preceding embodiments, wherein R¹ is —O—CHF₂.

57. The compound of any one of the preceding embodiments, wherein R¹ is —O—C₃₋₁₀ cycloalkyl, wherein the cycloalkyl is optionally substituted with one or more C₁₋₆ alkoxy or halogen.

58. The compound of any one of the preceding embodiments, wherein R¹ is —O-cyclopropyl or —O-cyclobutyl, wherein the cyclopropyl or cyclobutyl is optionally substituted with one or more fluorine or methoxy.

59. The compound of any one of the preceding embodiments, wherein R¹ is —O-3- to 8-membered heterocyclyl.

60. The compound of any one of the preceding embodiments, wherein R¹ is

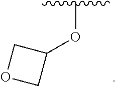 [SMILES: *OC1COC1]

61. The compound of any one of the preceding embodiments, wherein R¹ is —O—CH₂-(1,4-dioxan-2-yl) or —O—CH₂-tetrahydrofuran-2-yl.

62. The compound of any one of the preceding embodiments, wherein R¹ is —O—C₁₋₆ alkyl C₃₋₁₀ cycloalkyl optionally substituted with one or more halogen, cyano, or —OCH₃.

63. The compound of any one of the preceding embodiments, wherein R¹ is

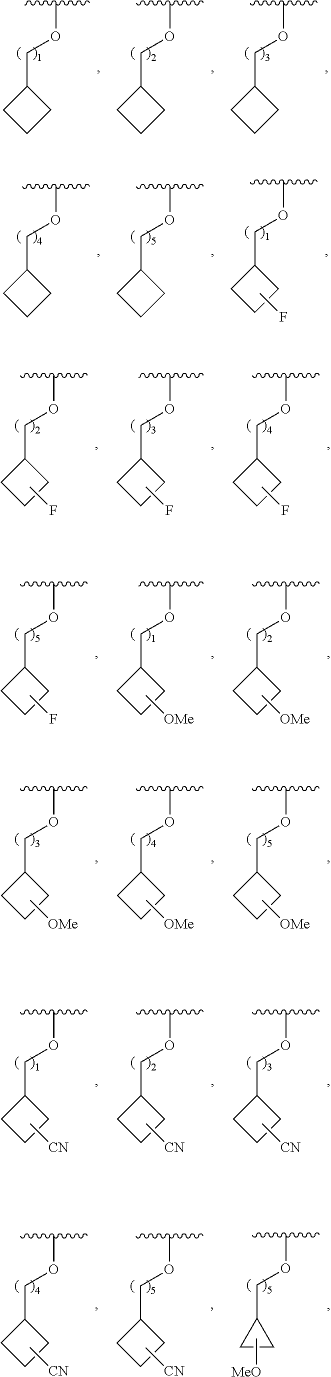

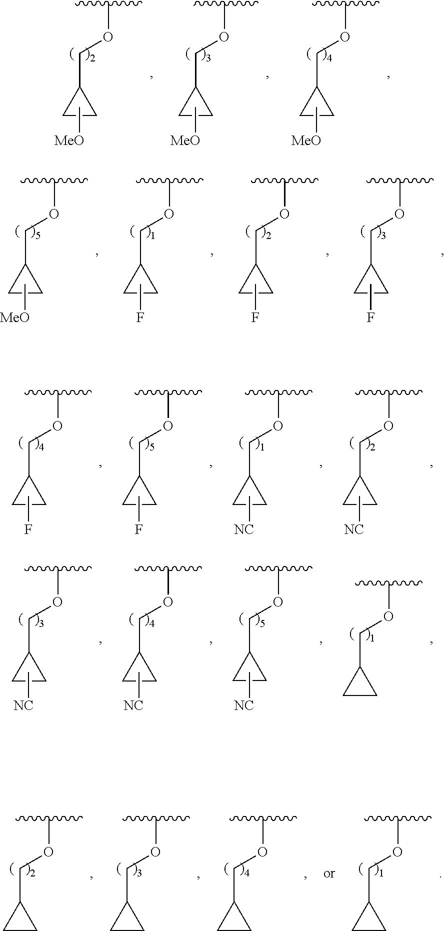

64. The compound of any one of the preceding embodiments, wherein R¹ is

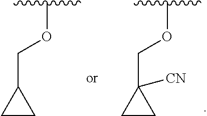 [SMILES: *OCC1CC1 ; *OCC1(C#N)CC1]

65. The compound of any one of the preceding embodiments, wherein R¹ is —O—C₁₋₆ alkyl-3- to 8-membered heterocyclyl.

66. The compound of any one of the preceding embodiments, wherein R¹ is

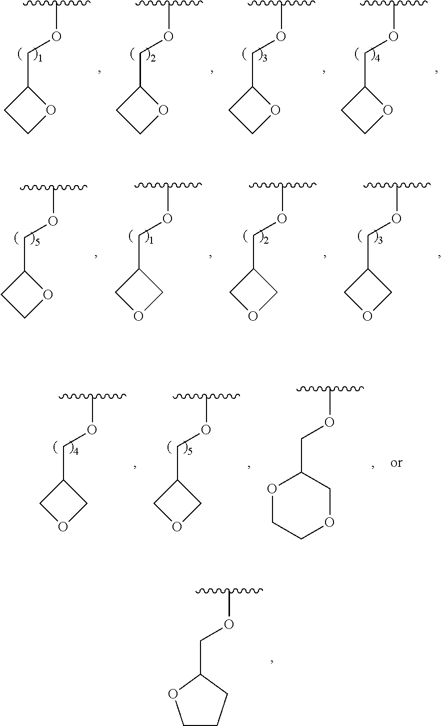

67. The compound of any one of the preceding embodiments, wherein R¹ is

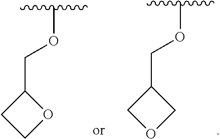 [SMILES: *OCC1CCO1 ; *OCC1COC1]

68. The compound of any one of the preceding embodiments, wherein R¹ is

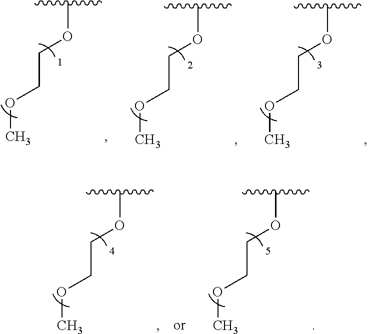

69. The compound of any one of the preceding embodiments, wherein R¹ is C₁₋₆ haloalkyl.

70. The compound of any one of the preceding embodiments, wherein R¹ is —CF₂—CH₃.

71. The compound of any one of the preceding embodiments, wherein R¹ is —O-(3- to 8-membered heterocyclyl).

72. The compound of any one of the preceding embodiments, wherein R¹ is —O-oxtenayl.

73. The compound of any one of the preceding embodiments, wherein R¹ is —O-oxetan-3-yl.

74. The compound of any one of the preceding embodiments, wherein R¹ is —O—(CH₂—CH(—OCH₃)—CH₂—O)₁₋₅—CH₃.

75. The compound of any one of the preceding embodiments, wherein R¹ is

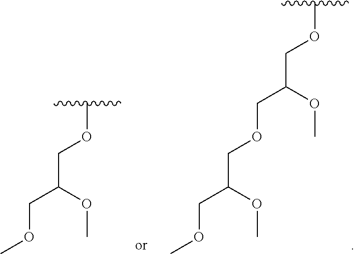

76. The compound of any one of the preceding embodiments, wherein R¹ is —NR⁸R⁹.

77. The compound of any one of the preceding embodiments, wherein R⁸ and R⁹ combine with the atom to which they are attached to form morpholine.

78. The compound of any one of the preceding embodiments, wherein R¹ is —NH₂.

79. The compound of any one of the preceding embodiments, wherein R¹ is NR⁸R⁹, wherein R⁸ is H and R⁹ is C₁₋₆ alkyl optionally substituted with oxo.

80. The compound of any one of the preceding embodiments, wherein R² is C₃₋₁₀ cycloalkyl optionally substituted with one or more —CN, C₁₋₆ haloalkyl, or C₁₋₆ alkyl optionally substituted with one or more —CN.

81. The compound of any one of the preceding embodiments, wherein R² is cyclopropyl optionally substituted with one or more —CN, C₁₋₆ haloalkyl, or C₁₋₆ alkyl optionally substituted with one or more —CN.

82. The compound of any one of the preceding embodiments, wherein R² is

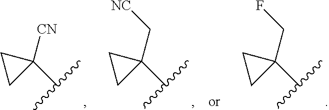 [SMILES: *C1(C#N)CC1 ; *C1(CC#N)CC1 ; *C1(CF)CC1]

83. The compound of any one of the preceding embodiments, wherein R² is 4 or 5-membered heterocyclyl comprising at least one oxygen or at least one sulfur atom, wherein the 4- or 5-membered heterocyclyl is optionally substituted with one or more oxo,

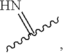 [SMILES: *=N]

or C₁₋₆ alkyl.

84. The compound of any one of the preceding embodiments, wherein R² is (i) thietane optionally substituted with one or more oxo or

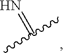 [SMILES: *=N]

(ii) oxetane, or (iii) tetrahydrofuran optionally substituted with one or more C₁₋₆ alkyl.

85. The compound of any one of the preceding embodiments, wherein R² is

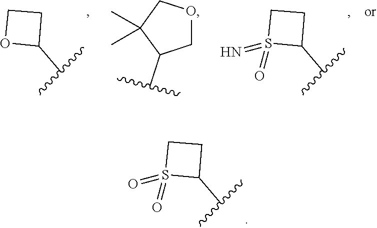

86. The compound of any one of the preceding embodiments, wherein R² is 5-membered heteroaryl, comprising 1 or 2, or 3 heteroatoms independently selected from N, and S, wherein at least one heteroatom of R⁵ is S.

87. The compound of any one of the preceding embodiments, wherein R² is thiazole.

88. The compound of any one of the preceding embodiments, wherein R² is

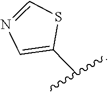 [SMILES: *c1cncs1]

89. The compound of any of the preceding embodiments, wherein R² is H.

90. The compound of any one of the preceding embodiments, wherein R² is —OCH₃.

91. The compound of any one of the preceding embodiments, wherein n is 1.

92. The compound of any one of the preceding embodiments, wherein X¹ is

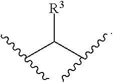

93. The compound of any one of the preceding embodiments, wherein X¹ is

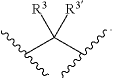

94. The compound of any one of the preceding embodiments, wherein R³ and R^{3′} independently are CH₃, CD₃, deuterium, or hydrogen.

95. The compound of any one of the preceding embodiments, wherein X¹ is

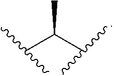

96. The compound of any one of the preceding embodiments, wherein X¹ is

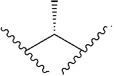

97. The compound of any one of the preceding embodiments, wherein X¹ is

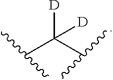 [SMILES: *C(*)([2H])[2H]]

98. The compound of any one of the preceding embodiments, wherein X¹ is

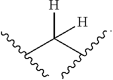 [SMILES: *C*]

99. The compound of any one of the preceding embodiments, wherein Ring A is

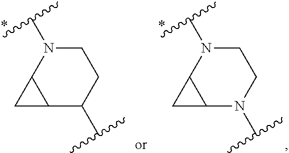 [SMILES: *C1CCN(*)C2CC12 ; *N1CCN(*)C2CC21]

wherein * indicates attachment to X¹.

100. The compound of any one of the preceding embodiments, wherein Ring A is

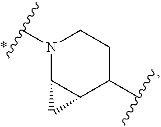 [SMILES: *C1CCN(*)[C@H]2C[C@@H]12]

wherein * indicates attachment to X¹.

101. The compound of any one of the preceding embodiments, wherein Ring A is

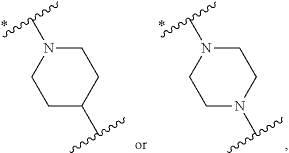 [SMILES: *C1CCN(*)CC1 ; *N1CCN(*)CC1]

wherein * indicates attachment to X¹.

102. The compound of any one of the preceding embodiments, wherein Ring A is

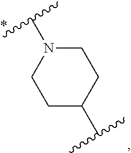 [SMILES: *C1CCN(*)CC1]

wherein * indicates attachment to X¹.

103. The compound of any one of the preceding embodiments, wherein Ring A is

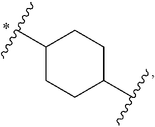 [SMILES: *C1CCC(*)CC1]

wherein * indicates attachment to X¹.

104. The compound of any one of the preceding embodiments, wherein Ring A is

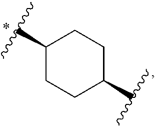 [SMILES: *[C@H]1CC[C@@H](*)CC1]

wherein * indicates attachment to X¹.

105. The compound of any one of the preceding embodiments, wherein Ring A is

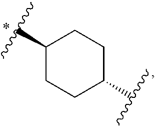 [SMILES: *[C@H]1CC[C@H](*)CC1]

wherein * indicates attachment to X¹.

106. The compound of any one of the preceding embodiments, wherein Ring A is phenyl optionally substituted with one or more halogen or C₁₋₆ alkyl.

107. The compound of any one of the preceding embodiments, wherein Ring A is

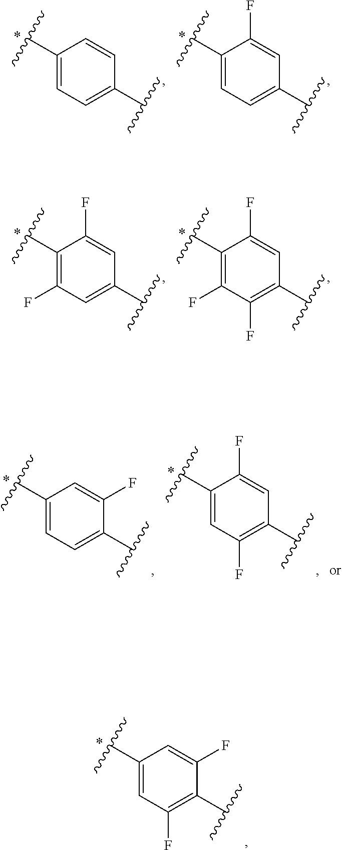

wherein [symbol] indicates attachment to X¹.

108. The compound of any one of the preceding embodiments, wherein Ring A is 6-membered heteroaryl optionally substituted with one or more halogen.

109. The compound of any one of the preceding embodiments, wherein Ring A is

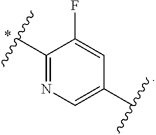 [SMILES: *c1cnc(*)c(F)c1]

110. The compound of any one of the preceding embodiments, wherein Ring B is a 6-membered heteroarylene comprising nitrogen optionally substituted with one or more halogen.

111. The compound of any one of the preceding embodiments, wherein Ring B is pyridinylene or pyrimidinylene optionally substituted with one or more halogen.

112. The compound of any one of the preceding embodiments, wherein Ring B is

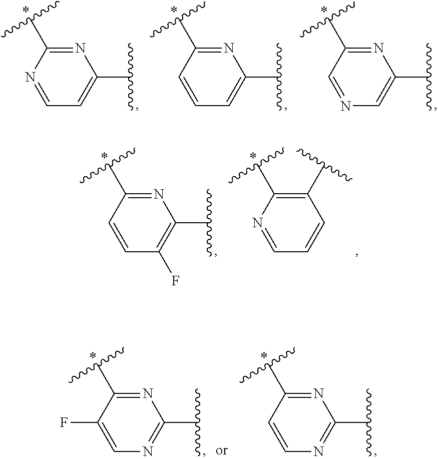

wherein * indicates attachment to Ring A or L′.

113. The compound of any one of the preceding embodiments, wherein Ring B is

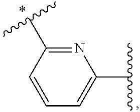 [SMILES: *c1cccc(*)n1]

wherein * indicates attachment to Ring A or L′.

114. The compound of any one of the preceding embodiments, wherein Ring B is a 9-membered heterocycylene comprising two oxygen atoms optionally substituted with one or more C₁₋₆ alkyl.

115. The compound of any one of the preceding embodiments, wherein Ring B is benzoidoxolylene optionally substituted with one or more C₁₋₆ alkyl.

116. The compound of any one of the preceding embodiments, wherein Ring B is

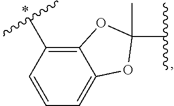 [SMILES: *c1cccc2c1OC(*)(C)O2]

wherein * indicates attachment to Ring A or L′.

117. The compound of any one of the preceding embodiments, wherein Ring B is a 10-membered heterocycylene.

118. The compound of any one of the preceding embodiments, wherein Ring B is a 10-membered heterocyclene comprising two oxygen atoms.

119. The compound of any one of the preceding embodiments, wherein Ring B is a 10-membered heterocyclene comprising one oxygen atom and one nitrogen atom.

120. The compound of any one of the preceding embodiments, wherein Ring B is benzodioxanylene.

121. The compound of any one of the preceding embodiments, wherein Ring B is

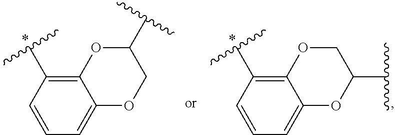 [SMILES: *c1cccc2c1OC(*)CO2 ; *c1cccc2c1OCC(*)O2]

wherein * indicates attachment to Ring A or L′.

122. The compound of any one of the preceding embodiments, wherein Ring B is

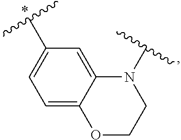 [SMILES: *c1ccc2c(c1)N(*)CCO2]

wherein * indicates attachment to Ring A or L′.

123. The compound of any one of the preceding embodiments, wherein Ring B is a phenylene optionally substituted with halogen, or C₁₋₆ alkoxy.

124. The compound of any one of the preceding embodiments, wherein Ring B is

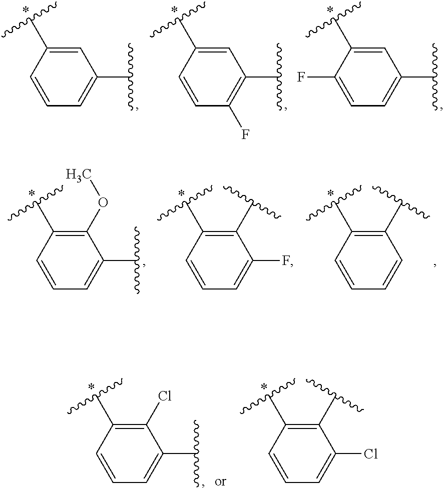

wherein * indicates attachment to Ring A or L′.

125. The compound of any one of the preceding embodiments, wherein Ring B is a 3-10 membered heterocycylene.

126. The compound of any one of the preceding embodiments, wherein Ring B is

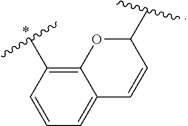 [SMILES: *c1cccc2c1OC(*)C=C2]

127. The compound of any one of the preceding embodiments, wherein Ring B is

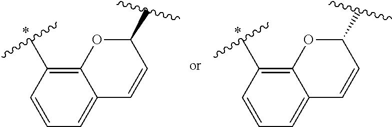 [SMILES: *c1cccc2c1O[C@@H](*)C=C2 ; *c1cccc2c1O[C@H](*)C=C2]

wherein * indicates attachment to Ring A or L′.

128. The compound of any one of the preceding embodiments, wherein L is a bond.

129. The compound of any one of the preceding embodiments, wherein L is *—O—CH₂—, *—O—CD₂-, or *—CH₂—O—, wherein * indicates attachment to Ring B.

130. The compound of any one of the preceding embodiments, wherein L is

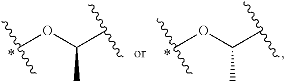

wherein * indicates attachment to Ring B.

131. The compound of any one of the preceding embodiments, wherein Ring C is phenyl optionally substituted with one or more —CN, halogen, —OCH₃,

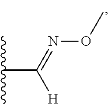 [SMILES: */C=N/OC]

or cyclopropyl.

132. The compound of any one the preceding embodiments, wherein Ring C is

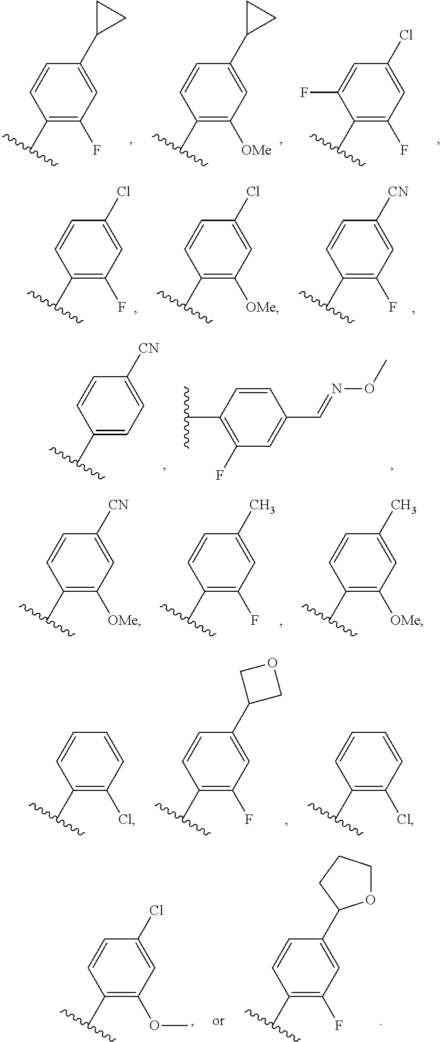

133. The compound of any one of the preceding embodiments, wherein Ring C is a 6-membered heteroaryl comprising a nitrogen atom optionally substituted with one or more halogen, —CN, —O—C₁₋₆ alkyl, —C(═O)—(C₃₋₁₀ cycloalkyl) or C₃₋₁₀ cycloalkyl.

134. The compound of any one of the preceding embodiments, wherein Ring C is pyridinyl optionally substituted with one or more —Cl, —F, —CN, —OCH₃, cyclopropyl,

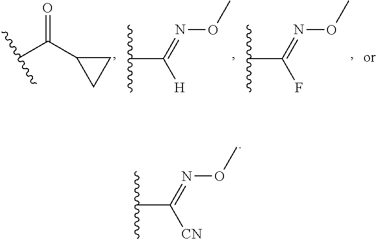

135. The compound of any one of the preceding embodiments, wherein Ring C is

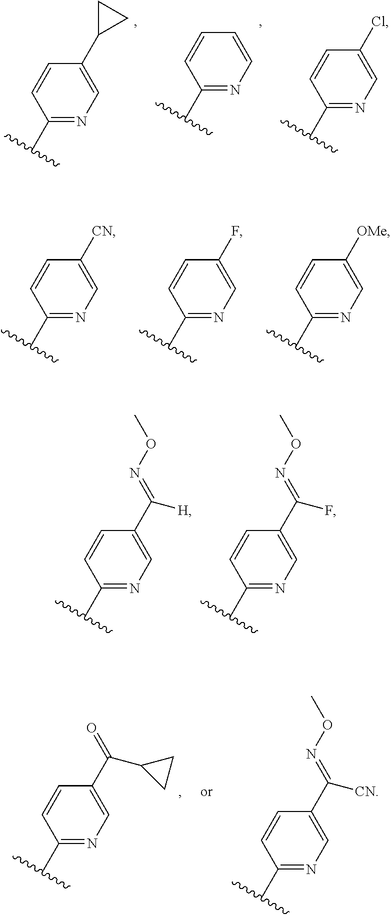

136. The compound of any one of the preceding embodiments, wherein Ring C is a bicylic 9- or 10-membered heteroaryl comprising two nitrogen atoms optionally substituted with one or more C₁₋₆ alkyl, or oxo.

137. The compound of any one of the preceding embodiments, wherein ring C is pyrazolopyridine, triazolopyridine, 2,3-dihydro-1H-pyrrolopyridine, 1,2-dihydrooxazolopyridine, 1,2,3,4-tetrahydronaphthyridine, 2,3-dihydro-pyridooxazine, 2,3-dihydro-1H-pyrrolopyridine, or 2,3-dihydrooxazolopyridine, wherein the pyrazolopyridine, triazolopyridine, 2,3-dihydro-1H-pyrrolopyridine, 1,2-dihydrooxazolopyridine, 1,2,3,4-tetrahydronaphthyridine, 2,3-dihydro-pyridooxazine, 2,3-dihydro-1H-pyrrolopyridine, or 2,3-dihydrooxazolopyridine is optionally substituted with one or more C₁₋₆ alkyl or oxo.

138. The compound of any one of the preceding embodiments, wherein Ring C is

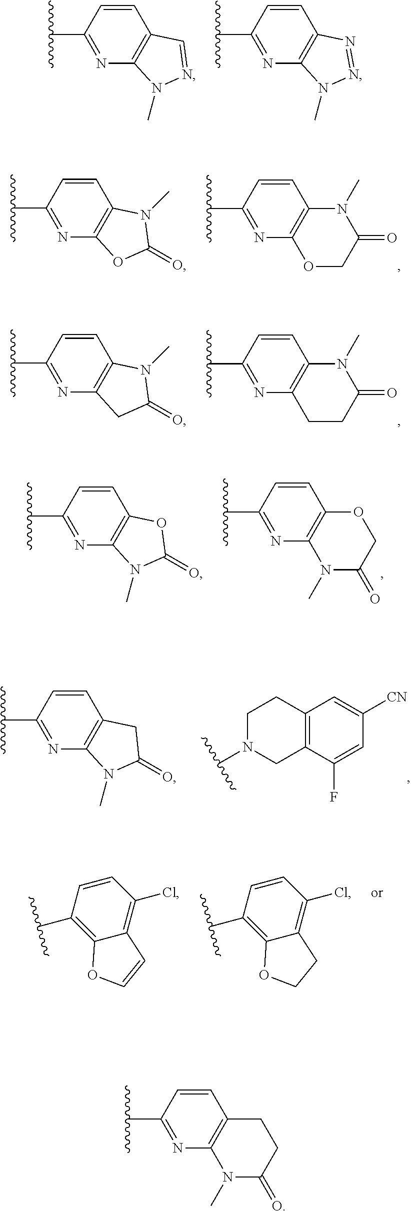

139. The compound of any one of the preceding embodiments, wherein

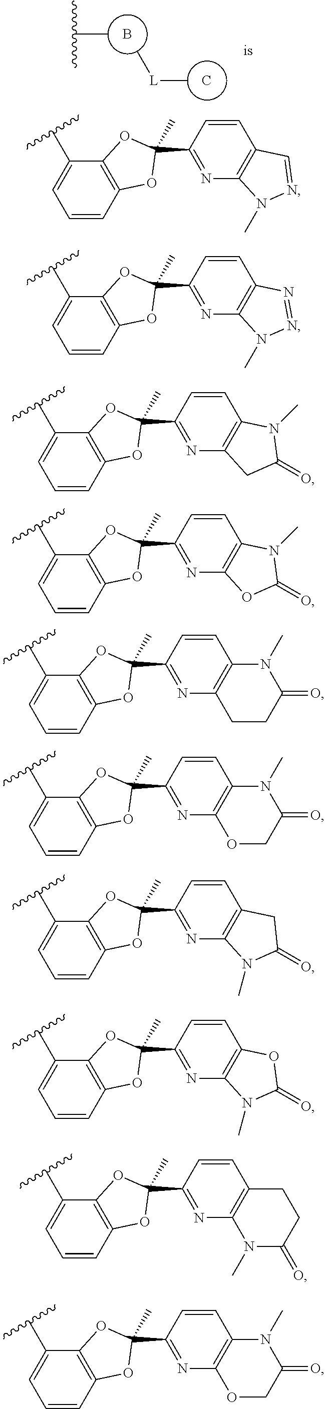

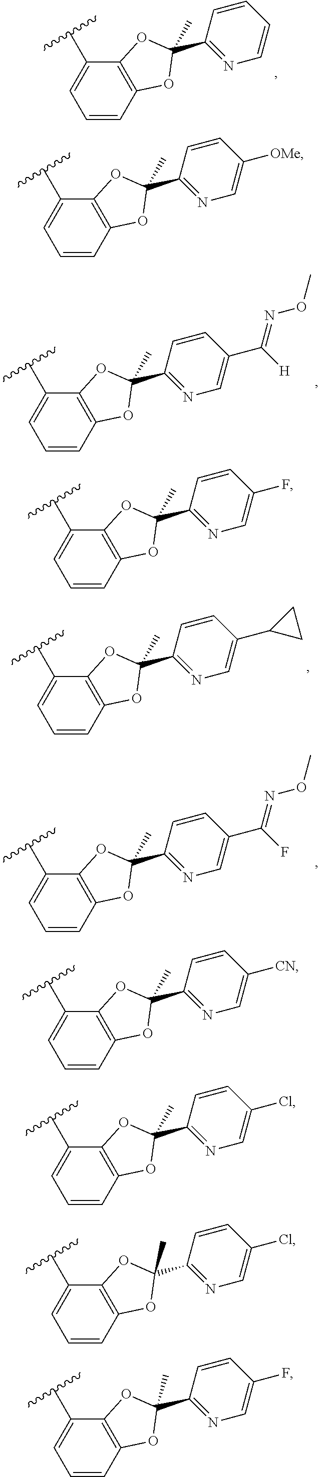

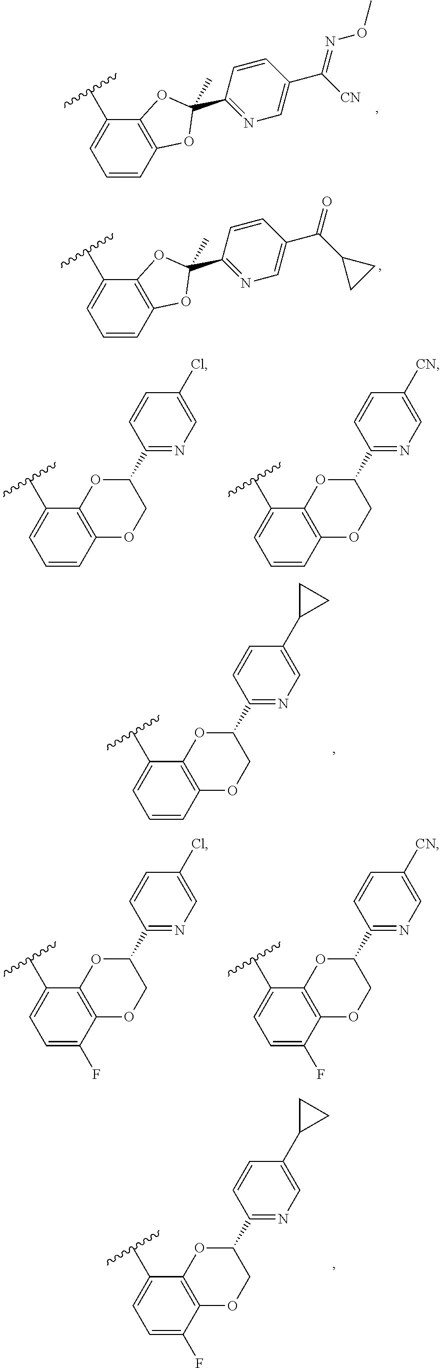

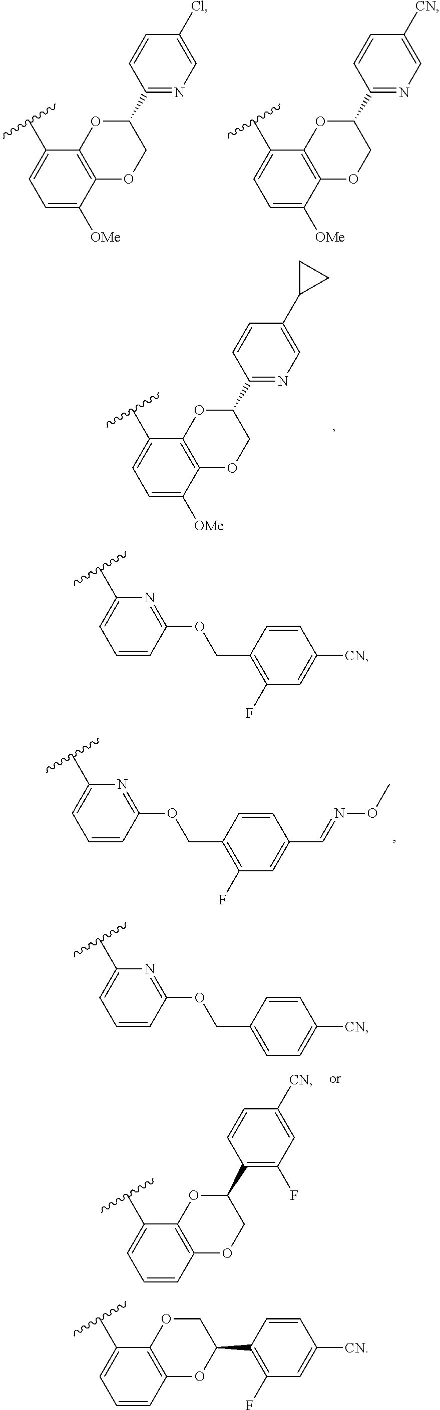

140. The compound of any one of the preceding embodiments, wherein

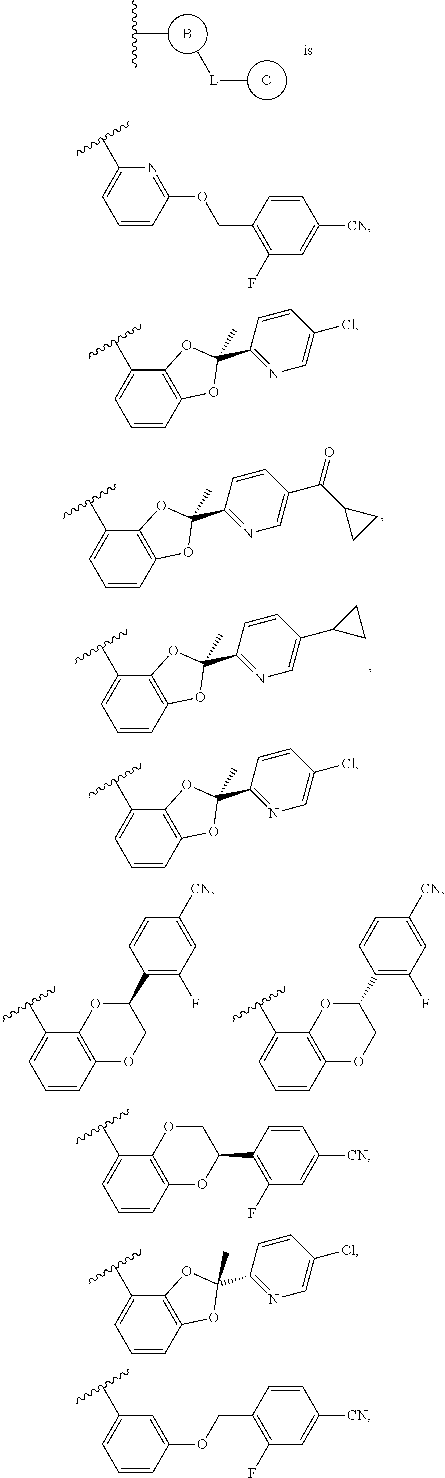

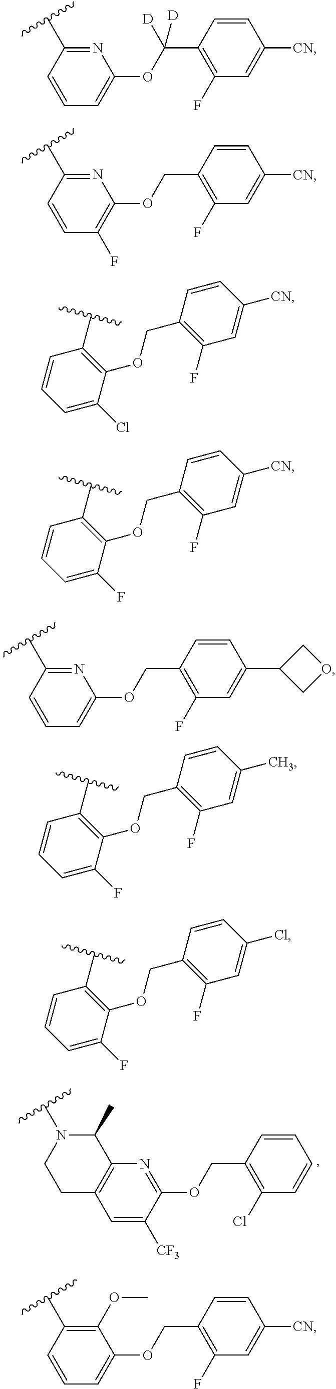

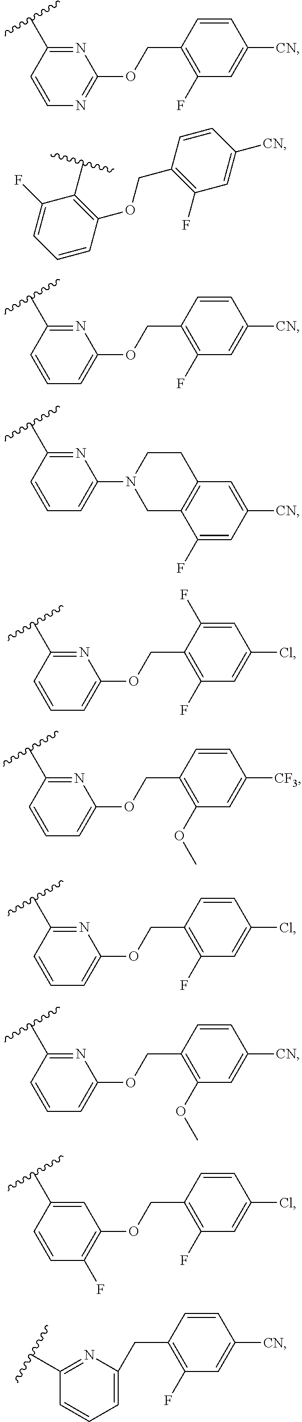

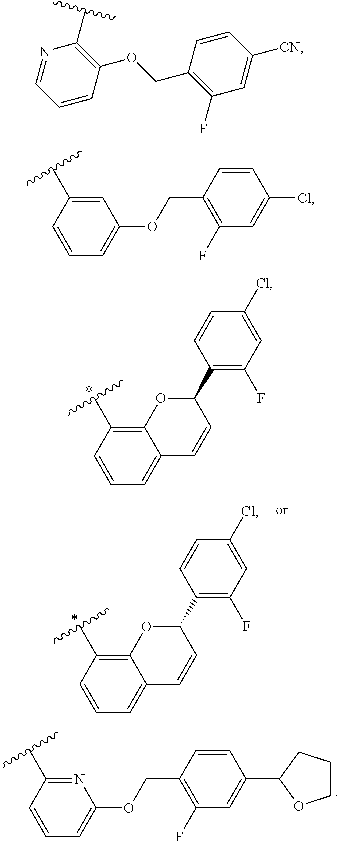

141. The compound of any one of the preceding embodiments, wherein R¹² is —C(O)OH.

142. The compound of any one of the preceding embodiments, wherein R¹² is

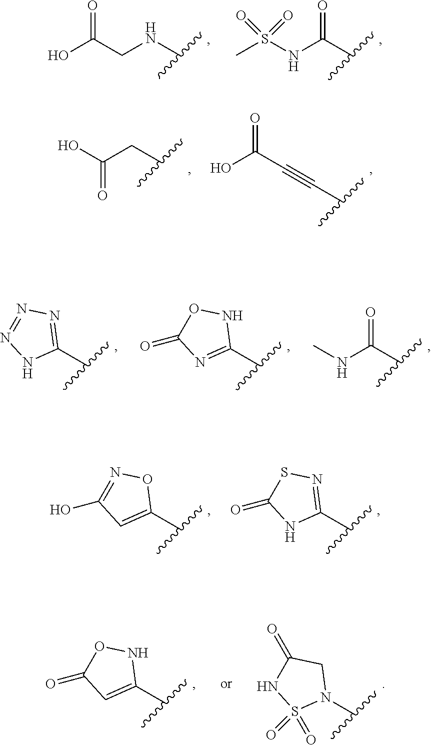

143. A compound or a pharmaceutically acceptable salt thereof, wherein the compound is selected form any one of the compounds in Table 1.

144. A pharmaceutical composition comprising the compound of any one of the preceding embodiments, or a pharmaceutically acceptable salt thereof, and a pharmaceutically acceptable excipient.

145. A method of treating a disease mediated by glucagon-like peptide-1 receptor (GLP-1R) in an individual in need thereof, comprising administering to the individual a therapeutically effective amount of the compound of any one of embodiments 1-143, or a pharmaceutically acceptable salt thereof, or the pharmaceutical composition of embodiment 144.

146. The method of embodiment 145, wherein the disease is a liver disease.

147. The method of embodiment 146, wherein the liver disease is primary biliary cirrhosis (PBC), primary sclerosing cholangitis (PSC), drug induced cholestasis, intrahepatic cholestasis of pregnancy, parenteral nutrition associated cholestasis (PNAC), bacterial overgrowth or sepsis associated cholestasis, autoimmune hepatitis, viral hepatitis, alcoholic liver disease, nonalcoholic fatty liver disease (NAFLD), nonalcoholic steatohepatitis (NASH), graft versus host disease, transplant liver regeneration, congenital hepatic fibrosis, choledocholithiasis, granulomatous liver disease, intra- or extrahepatic malignancy, Sjogren's syndrome, sarcoidosis, Wilson's disease, Gaucher's disease, hemochromatosis, or oti-antitrypsin deficiency.

148. The method of embodiment 145, wherein the disease is diabetes.

149. The method of embodiment 145, wherein the disease is a cardiometabolic disease.

150. The method of embodiment 145, wherein the disease is obesity.

151. Use of the compound of any one of embodiments 1-143, or a pharmaceutically acceptable salt thereof, in the manufacture of a medicament for treating a disease mediated by GLP-1R.

152. A method of decreasing food intake in an individual in need thereof, comprising administering to the individual a compound, or pharmaceutically acceptable salt thereof, of any one of embodiments 1-143 or the pharmaceutical composition of embodiment 144.

153. A method of increasing glucose tolerance in an individual in need thereof, comprising administering to the individual a compound, or pharmaceutically acceptable salt thereof, of any one of embodiments 1-139 or the pharmaceutical composition of embodiment 140.

154. A method of treating obesity in an individual in need thereof, comprising administering to the individual a compound, or pharmaceutically acceptable salt thereof, of any one of embodiments 1-143 or the pharmaceutical composition of embodiment 144.

155. Use of the compound of any one of embodiments 1-143, or a pharmaceutically acceptable salt thereof, or the pharmaceutical composition of embodiment 144, in the manufacture of a medicament for treating obesity.

156. The compound of any one of embodiments 1-143, or a pharmaceutically acceptable salt thereof, or the pharmaceutical composition of embodiment 144, for use in treating obesity in an individual in need thereof.

1. A compound of Formula (I**): 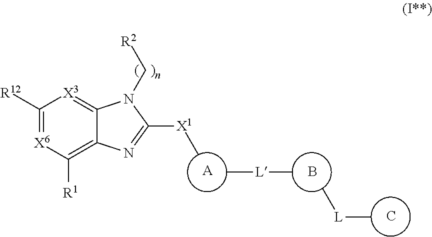
or a pharmaceutically acceptable salt thereof; wherein:
X³ is CR⁶ or N;
X⁶ is CR⁴ or N;
R′ is —C₁₋₆ haloalkyl, halogen, —O—X⁴, or —NR⁸R⁹, or R′ and R⁴, together with the atoms to which they are attached, combine to form a 5- or 6-membered heterocyclyl;
X⁴ is hydrogen, C₁₋₆ alkyl, C₁₋₆ heteroalkyl, C₁₋₆ haloalkyl, —(C₁₋₆ alkyl)-(C₃₋₁₀ cycloalkyl), or —(C₁₋₆ alkyl)-(3- to 8-membered heterocyclyl), 3- to 8-membered heterocyclyl, —(CH₂CH₂—O)₁₋₅—CH₃, —(CH₂—CH(—OCH₃)—CH₂—O)₁₋₅—CH₃, C₃₋₁₀ cycloalkyl, or C₆₋₁₀ aryl, wherein the alkyl, heteroalkyl, or haloalkyl group is optionally substituted with one or more deuterium, C₁₋₆ alkoxy, hydroxyl, —CN, or oxo, and the cycloalkyl, heterocyclyl, or aryl group is optionally substituted with one or more halogen, C₁₋₆ alkoxy, or —CN;
R⁶ is hydrogen, halogen, or —O—R⁷;
wherein R⁷ and R², together with the atoms to which they are attached, combine to form a 6-membered heterocyclyl;
R⁸ and R⁹ each independently are selected from hydrogen, C₁₋₆ alkyl, or —S(O)₂—C₁₋₆ alkyl, or R⁸ and R⁹, together with the atoms to which they are attached, combine to form a 6-membered heterocycyl; wherein the C₁₋₆ alkyl is optionally substituted by one or more oxo;
n is 0, 1, 2, 3, 4, 5, or 6;
R² is:
hydrogen;
C₁₋₆ alkyl optionally substituted with deuterium;
C₁₋₆ haloalkyl;
—(O)—C₁₋₆ alkyl;
—CN;
a C₃₋₁₀ cycloalkyl optionally substituted with one or more —CN, C₁₋₆ haloalkyl, or C₁₋₆ alkyl optionally substituted with one or more —CN;
a 4 or 5-membered heterocyclyl comprising at least one oxygen or at least one sulfur atom, wherein the 4- or 5-membered heterocyclyl is optionally substituted with one or more oxo, 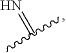 [SMILES: *=N]
or C₁₋₆ alkyl;
a 5-membered heteroaryl, comprising 1 or 2 heteroatoms independently selected from N, and S, wherein at least one heteroatom of R⁵ is S; or
R² and R⁷, together with the atoms to which they are attached, combine to form a 5- or 6-membered heterocyclyl;
R⁴ is halogen, hydrogen, —C(O)OH, or —O—R⁸,
wherein R⁸ and R¹, together with the atoms to which they are attached, combine to form a 6-membered heterocyclyl;
R¹² is hydrogen, —C(O)OH, —C(O)NR^{N12}R^{N12′}, —C(O)NR¹²S(O)₂R^{12′}, —(C₂₋₆ alkynylene)-C(O)OH, —(C₁₋₆ alkylene)-C(O)OH, —NR^{N12}—(C₁₋₆ alkylene)-C(O)OH, 5-10 membered heteroaryl or 5- to 10-membered heterocyclyl optionally substituted with one or more oxo, C₁₋₆ alkyl or C₁₋₆ haloalkyl;
R^{N12} and R^{N12′} independently are H or C₁₋₆ alkyl;
X¹ is 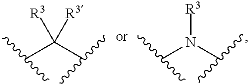
wherein R³ and R^{3′} independently are H, D or C₁₋₆ alkyl, wherein the C₁₋₆ alkyl is optionally substituted with deuterium;
Ring A is 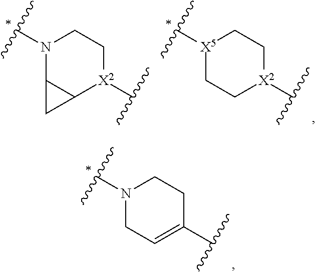
phenyl optionally substituted with one or more halo or C₁₋₆ alkyl, or 6-membered heteroaryl optionally substituted with one or more halo or C₁₋₆ alkyl;
wherein * indicates attachment to X¹,
X⁵ is CR³ or N, and
X² is CR³ or N;
L′ is a bond or —O—;
Ring B is a C₆₋₁₀ arylene, a 5-10 membered heteroarylene, or a 3-10 membered heterocyclene, wherein the C₆₋₁₀ arylene, 5-10 membered heteroarylene, or 3-10 membered heterocyclene is optionally substituted with one or more oxo, C₁₋₆ alkyl, C₁₋₆ alkoxy, or halogen;
L is a bond, *—(C₁₋₆ alkylene)-, *—NR^{L}—(C₁₋₆ alkyl), *—O—(C₁₋₆ alkyl)-, or *—(C₁₋₆ alkyl)-O—, wherein * indicates attachment to Ring B and the C₁₋₆ alkylene or C₁₋₆ alkyl is optionally substituted with deuterium;
wherein R^{L} is H or C₁₋₆ alkyl; and
Ring C is:
a 6-membered aryl optionally substituted with one or more C₃₋₁₀ cycloalkyl, C₁₋₆ alkyl, C₁₋₆ haloalkyl, 3-10 membered heterocyclyl, halogen, C₁₋₆ alkoxy, C₁₋₆ haloalkoxy, —CN, C₃₋₁₀ cycloalkyl, or —C(O)NR′₂;
wherein R′ is H or C₁₋₆ alkyl
a 6-membered heteroaryl comprising a nitrogen atom optionally substituted with one or more halogen, —CN, C₁₋₆ haloalkyl, —O—C₁₋₆ alkyl, C₃₋₁₀ cycloalkyl, —C(═O)—(C₃₋₁₀ cycloalkyl), 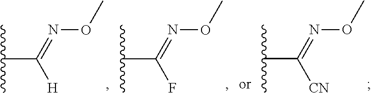
or
a bicylic 9- or 10-membered heteroaryl or heterocyclyl optionally substituted with one or more C₁₋₆ alkyl, halogen, —CN, or oxo.

2-3. (canceled)

4. The compound of claim 1, wherein the compound of Formula (I**) is of Formula (II**) 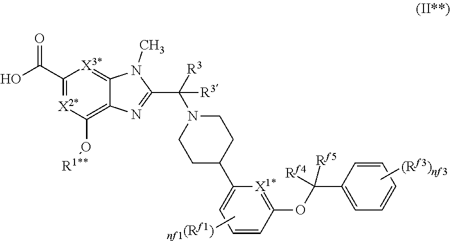
or a pharmaceutically acceptable salt thereof; wherein
R^{f4} and R^{f5} are each independently selected from C₁₋₆ alkyl, H and D
nf1 is 0, 1, 2, 3, or 4;
nf3 is 0, 1, 2, 3, 4, or 5;
each R^{f1} is halogen;
R³ and R^{3′} independently are H or D;
X¹* is N or CR^{f1};
X^{2*} and X^{3*} independently are CH or CF;
each R^{f3} is independently selected from halogen, —CN, C₁₋₆ alkyl, C₁₋₆ haloalkyl, C₁₋₆ alkoxy, or —C(O)N(R^{f3′})₂,
each R^{f3′} is independently selected from H or C₁₋₆ alkyl; and
R^{1**} is H or C₁₋₂ alkyl optionally substituted with one or more deuterium or halogen.

5. The compound of claim 1, wherein the compound of Formula (I**) is of Formula (II*) 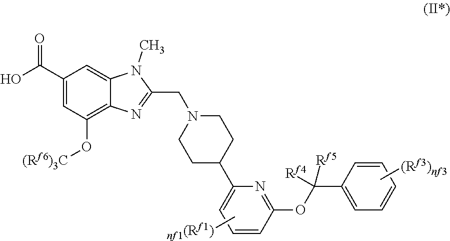
or a pharmaceutically acceptable salt thereof;
each R^{f1} is independently selected from halogen
R^{f4} and R^{f5} are each independently selected from C₁₋₆ alkyl, H and D
nf1 is 0, 1, 2, or 3;
nf3 is 0, 1, 2, 3, 4, or 5;
each R^{f1} is halogen;
each R^{f3} is independently selected from halogen, —CN, C₁₋₆ alkyl, C₁₋₆ haloalkyl, C₁₋₆ alkoxy, or —C(O)N(R^{f3′})₂,
each R^{f3′} is independently selected from H or C₁₋₆ alkyl;
each R^{f6} is independently selected from H, D or F.

6. The compound of claim 1, wherein;
(i) R¹ is —O—C₁₋₆ alkyl, wherein the alkyl group is linear and is optionally substituted with one or more deuterium, —CN or —O—C₁₋₆ alkyl, or wherein the alkyl group is branched and is optionally substituted with one or more —CN;
(ii) R¹ is —O—C₁₋₆ haloalkyl;
(iii) R¹ is —O—C₃₋₁₀ cycloalkyl optionally substituted with one or more C₁₋₆ alkoxy or halogen, —O-3- to 8-membered heterocyclyl, or —O—C₁₋₆ alkyl C₃₋₁₀ cycloalkyl optionally substituted with one or more halogen, cyano, or —OCH₃; or
(iv) R¹ is —NR⁸R⁹.

7. (canceled)

8. The compound of claim 1, wherein R¹ is —O—C₁₋₂ alkyl, wherein the alkyl is optionally substituted with one or more deuterium or fluorine.

9-10. (canceled)

11. The compound of claim 1, any one of the preceding claims, wherein:
(i) R⁸ and R⁹ combine with the atom to which they are attached to form morpholine;
(ii) R¹ is NH₂; or
(iii) R⁸ is H and R⁹ is C₁₋₆ alkyl optionally substituted with oxo.

12. The compound of claim 1, any one of the preceding claims, wherein:
(i) R² is C₃₋₁₀ cycloalkyl optionally substituted with one or more —CN, C₁₋₆ haloalkyl, or C₁₋₆ alkyl optionally substituted with one or more —CN;
(ii) R² is 4 or 5-membered heterocyclyl comprising at least one oxygen or at least one sulfur atom, wherein the 4- or 5-membered heterocyclyl is optionally substituted with one or more oxo, 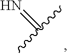 [SMILES: *=N]
or C₁₋₆ alkyl;
(iii) R² is 5-membered heteroaryl, comprising 1 or 2, or 3 heteroatoms independently selected from N, and S, wherein at least one heteroatom of R⁵ is S; or
(iv) R² is H or —OCH₃.

13-15. (canceled)

16. The compound of claim 1, wherein n is 1.

17. The compound of claim 1, wherein X¹ is 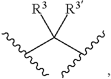
wherein R³ and R^{3′} independently are CH₃, CD₃, deuterium, or hydrogen.

18. The compound of any claim 1, wherein Ring A is 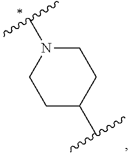 [SMILES: *C1CCN(*)CC1]
wherein * indicates attachment to X¹.

19. The compound of claim 1, wherein Ring A is 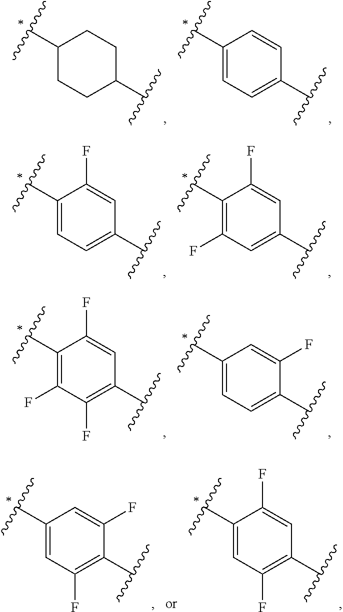
wherein * indicates attachment to X¹.

20. The compound of claim 1, wherein Ring B is a 6-membered heteroarylene comprising nitrogen optionally substituted with one or more halogen.

21. The compound of claim 1, wherein Ring B is 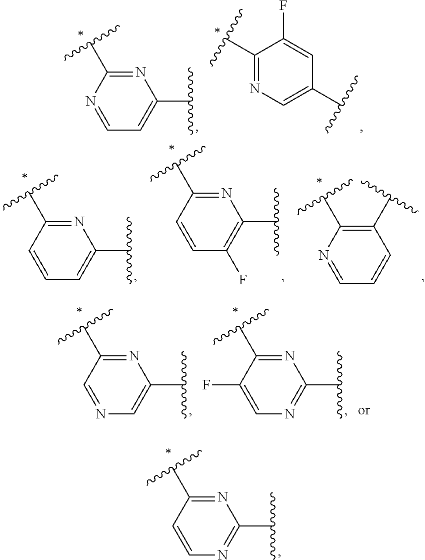
wherein * indicates attachment to Ring A or L′.

22. The compound of claim 1, wherein Ring B is a 9-membered heterocycylene comprising two oxygen atoms optionally substituted with one or more C₁₋₆ alkyl, or a 10-membered heterocycylene.

23. (canceled)

24. The compound of claim 1, wherein Ring B is a phenylene optionally substituted with halogen or C₁₋₆ alkoxy.

25. The compound of claim 1, wherein Ring B is 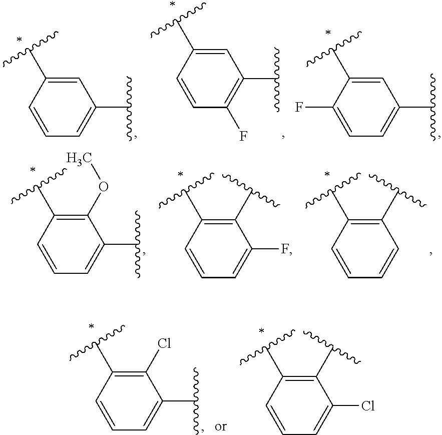
wherein * indicates attachment to Ring A or L′.

26. The compound of claim 1, wherein L is a bond.

27. The compound of claim 1, wherein:
(i) L is *—O—CH₂—, *—O—CD₂-, or *—CH₂—O—, wherein * indicates attachment to Ring B; or
(ii) L is 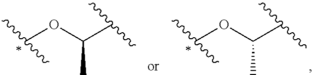
wherein * indicates attachment to Ring B.

28. (canceled)

29. The compound of claim 1, wherein Ring C is phenyl optionally substituted with one or more —CN, halogen, —OCH₃, 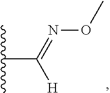 [SMILES: */C=N/OC]
or cyclopropyl.

30. The compound of claim 1, wherein Ring C is

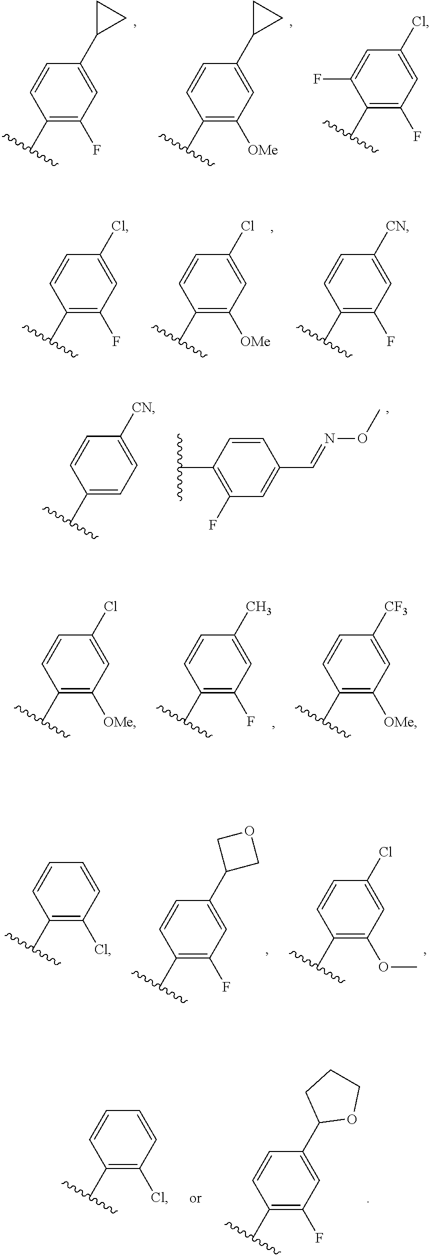

31. The compound of claim 1, wherein Ring C a 6-membered heteroaryl comprising a nitrogen atom optionally substituted with one or more halogen, —CN, —O—C₁₋₆ alkyl, —C(═O)—(C₃₋₁₀ cycloalkyl) or C₃₋₁₀ cycloalkyl.

32. (canceled)

33. The compound of claim 1, wherein Ring C is

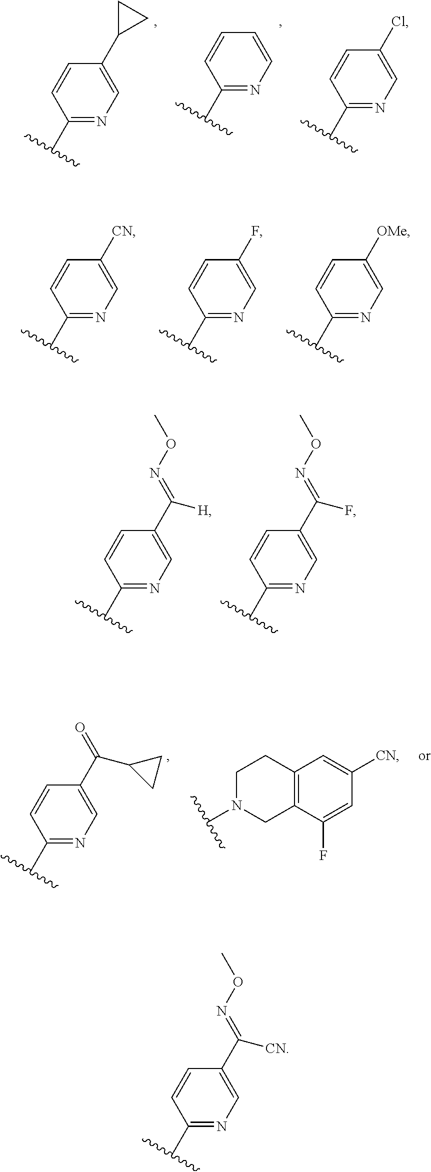

34. The compound of claim 1, wherein R¹² is —COOH.

35. (canceled)

36. A compound or a pharmaceutically acceptable salt thereof, wherein the compound is selected from any one of the compounds in Table 1.

37. A pharmaceutical composition comprising the compound of claim 1, or a pharmaceutically acceptable salt thereof, and a pharmaceutically acceptable excipient.

38. A method of treating a disease mediated by glucagon-like peptide-1 receptor (GLP-1R) in an individual in need thereof, comprising administering to the individual a therapeutically effective amount of the compound of claim 1, or a pharmaceutically acceptable salt thereof.

39. (canceled)

40. The method of claim 38, wherein the disease is primary biliary cirrhosis (PBC), primary sclerosing cholangitis (PSC), drug induced cholestasis, intrahepatic cholestasis of pregnancy, parenteral nutrition associated cholestasis (PNAC), bacterial overgrowth or sepsis associated cholestasis, autoimmune hepatitis, viral hepatitis, alcoholic liver disease, nonalcoholic fatty liver disease (NAFLD), nonalcoholic steatohepatitis (NASH), graft versus host disease, transplant liver regeneration, congenital hepatic fibrosis, choledocholithiasis, granulomatous liver disease, intra- or extrahepatic malignancy, Sjogren's syndrome, sarcoidosis, Wilson's disease, Gaucher's disease, hemochromatosis, or oti-antitrypsin deficiency.

41. The method of claim 38, wherein the disease is diabetes.

42. The method of claim 38, wherein the disease is a cardiometabolic disease.

43. The method of claim 38, wherein the disease is obesity.

44. A method of decreasing food intake in an individual in need thereof, comprising administering to the individual a compound, or pharmaceutically acceptable salt thereof, of claim 1.

45. A method of increasing glucose tolerance in an individual in need thereof, comprising administering to the individual a compound, or pharmaceutically acceptable salt thereof, of claim 1.

46-61. (canceled)

62. A method of treating obesity in an individual in need thereof, comprising administering to the individual a compound, or pharmaceutically acceptable salt thereof, of claim 1.

63-64. (canceled)

65. A kit comprising the compound of claim 1, or a pharmaceutically acceptable salt thereof.

66. A method of synthesizing a compound of claim 1, comprising the step of contacting a compound of Formula E-4: 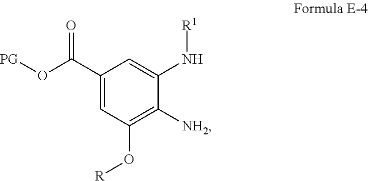
with a compound of the structure: 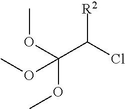
To produce a compound of Formula E-5: 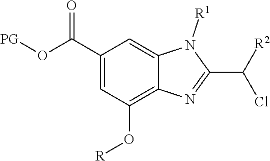
wherein:
PG is methyl or tert-butyl;
R is H or C₁₋₂ alkyl optionally substituted with one or more deuterium or halogen;
R¹ is C₁₋₆ alkyl; and
R² is H, D or C₁₋₆ alkyl, wherein the C₁₋₆ alkyl is optionally substituted with deuterium.
